(19) United States
(12) Patent Application Publication
(10) Pub. No.: US 2026/0125386 A1
(43) Pub. Date: May 7, 2026

(54) COMPOSITIONS AND METHODS FOR INHIBITION OF KRAS

(71) Applicants: TheRas, Inc., South San Francisco, CA (US); Leidos Biomedical Research, Inc., Frederick, MD (US); Lawrence Livermore National Security, LLC, Livermore, CA (US)

(72) Inventors: Zuhui Zhang, Millbrae, CA (US); Bin Wang, Fort Collins, CO (US); Eli Wallace, Loveland, CO (US); Rui Xu, Dallas, TX (US); Paul Wehn, New Haven, CT (US); Yue Yang, Foster City, CA (US); Felice Lightstone, Fremont, CA (US); Jun Pei, Livermore, CA (US); Anna Elzbieta Maciag, Frederick, MD (US); David Michael Turner, Frederick, MD (US); Dhirendra Kumar Simanshu, Frederick, MD (US); Albert Hay Wah Chan, Frederick, MD (US); Christopher John Brassard, Frederick, MD (US); Tao Liao, Frederick, MD (US)

(21) Appl. No.: 19/439,970

(22) Filed: Jan. 5, 2026

Related U.S. Application Data
(63) Continuation of application No. 19/100,853.
(60) Provisional application No. 63/395,699, filed on Aug. 5, 2022.
(60) Provisional application No. 63/386,285, filed on Dec. 6, 2022.
(60) Provisional application No. 63/496,873, filed on Apr. 18, 2023.

(51) Int. Cl. C07D 487/04; A61K 31/517; A61K 31/519; A61K 31/5365; A61K 31/5377; A61K 31/538; A61K 31/5383; A61K 31/5386; A61K 31/541; A61K 31/547; A61K 31/551; A61K 31/553; A61K 31/554; A61P 35/00; C07B 59/00; C07D 409/14; C07D 413/14; C07D 417/14; C07D 487/10; C07D 498/08; C07D 519/00

(52) U.S. Cl. CPC C07D 487/04; A61K 31/517; A61K 31/519; A61K 31/5365; A61K 31/5377; A61K 31/538; A61K 31/5383; A61K 31/5386; A61K 31/541; A61K 31/547; A61K 31/551; A61K 31/553; A61K 31/554; A61P 35/00; C07B 59/002; C07D 409/14; C07D 413/14; C07D 417/14; C07D 487/10; C07D 498/08; C07D 519/00

(57) ABSTRACT

Provided herein are compounds, or salts, esters, tautomers, prodrugs, zwitterionic forms, or stereoisomers thereof, as well as pharmaceutical compositions comprising the same. Also provided herein are methods of using the same in modulating (e.g., inhibiting) KRAS (e.g., KRAS having a Q61H, G12D, G12V, G12C, G12S, G12A, G12R, or G13D mutation or wild-type KRAS) and treating diseases or disorders such as cancers in subjects in need thereof.

### RELATED APPLICATIONS

This application claims priority to and benefit of U.S. Application No. 63/395,699, filed Aug. 5, 2022, U.S. Application No. 63/386,285, filed Dec. 6, 2022, and U.S. Application No. 63/496,873, filed Apr. 18, 2023, the entire contents of each of which are hereby incorporated by reference.

### STATEMENT AS TO RIGHTS TO INVENTIONS MADE UNDER FEDERALLY SPONSORED RESEARCH AND DEVELOPMENT

This invention was made with government support under (1) Contract No.: 75N91019D00024 awarded by the National Institutes of Health and (2) Contract No. DE-AC52-07NA27344 awarded by the United States Department of Energy. The government has certain rights in the invention.

### BACKGROUND

RAS protein functions as a molecular switch, cycling between inactive (“GDP-bound”) and active (“GTP-bound”) states. RAS signaling occurs through engagement with effector proteins that adapt the signaling cascades regulating tumor cell survival and proliferation. Aberrant activation of RAS by oncogenic mutations results in increased GTP-bound KRAS and constitutive downstream signaling.

RAS is the most frequently mutated oncogene. Activating mutations in KRAS occur in over 90% of pancreatic tumors. Mutated KRAS is also observed at high frequency in other common tumors, including colorectal cancer (˜44%) and non-small cell lung cancer (NSCLC; ˜20-30%). Cancer-associated mutations in KRAS cluster in three hotspots (G12, G13, and Q61), with a majority (77%) of mutations causing single amino acid substitutions at G12. The KRAS missense mutation G12D is the most predominant variant in human malignancies (35%), followed by G12V (29%). Besides G12, the hotspots G13 and Q61 show mutation rates of 10% and 6% respectively.

The development of small molecule KRAS inhibitors has proven to be a challenge. Recent clinical development of covalent KRAS G12C inhibitors indicates potential of targeting the KRAS oncogenic protein directly. Results from clinical trials with the two covalent inhibitors, AMG510 (sotorasib) and MRTX849 (adagrasib), have been promising. These inhibitors demonstrated clinical activity primarily in NSCLC, where the KRAS G12C mutation frequency is highest. Unfortunately, they appeared less effective in KRAS G12C colorectal cancers. Both compounds only target the inactive (GDP-bound) form of KRAS G12C, and a lack of activity against active (GTP-bound) KRAS G12C may contribute to development of drug resistance. Moreover, these covalent inhibitors are limited to the specific G12C mutant that accounts for approximately 13% of all KRAS-driven cancers, leaving a large population of non-G12C KRAS cancers still undruggable. Therefore, KRAS therapeutics that target additional KRAS alterations or combinations thereof are an unmet clinical need. Pan-KRAS inhibitors hold promise for impact across the majority of KRAS mutant alleles, including the most prevalent G12D and G12V, or KRAS wildtype-amplified cancers.

KRAS is essential for mouse development, whereas NRAS and HRAS are dispensable. This requirement for KRAS creates toxicity concerns when targeting the wild-type KRAS protein. However, when KRAS is replaced with HRAS, mice are viable, which reduces toxicity concerns and suggests that in contrast to the pan-RAS inhibitors that could pose toxicity issues, KRAS isoform-specific inhibitors should be tolerated. If so, additional advantages of pan-KRAS inhibitors could come from targeting cancers with acquired resistance to KRAS G12C inhibitors. Recent reports provide insights into mechanisms of resistance to the KRAS G12C inhibitors in the clinic, suggesting restoration of RAS/MAPK as a driver of the resistance. Acquisition of a diverse set of mutations in response to KRAS G12C inhibitors in addition to activation of the KRAS wildtype allele through upstream RTK signaling has been shown. It is possible that direct pan-KRAS agents may suppress these events. Accordingly, there remains a need for allele-specific and pan-KRAS inhibitors that could be used to treat KRAS-driven cancers regardless of mutation status.

### SUMMARY

The present disclosure provides compounds, as well as compositions and kits comprising the same, and methods of using the same in the treatment of diseases and disorders such as cancers. The present disclosure provides compounds that may be capable of inhibiting one or more mutant forms of KRAS, such as KRAS having a G12D, G12V, G12C, G12S, G12A, G12R, Q61H, or G13D mutation, or wild-type KRAS. Such compounds may be considered pan-KRAS inhibitors. In some embodiments, the compounds provided herein may be capable of targeting both active GTP-bound protein and inactive GDP-bound protein, which inhibitors may provide therapeutic advantages over compounds capable of targeting only the inactive GDP-bound protein. In some embodiments, compounds provided herein have inhibitory activity against a KRAS protein comprising a glycine to aspartic acid, valine, cysteine, serine, alanine, or arginine mutation at codon 12 (i.e., a G12D, G12V, G12C, G12S, G12A, or G12R mutation); or a glycine to aspartic acid mutation at codon 13 (e.g., a G13D mutation); or a glutamine to histidine mutation at codon 61 (e.g., a Q61H mutation) in both its active and inactive conformations. In some embodiments, compounds provided herein have inhibitory activity against wild-type KRAS. In some embodiments, compounds provided herein are useful in the treatment of cancers, such as cancers characterized by KRAS proteins having a mutation at codon 12, such as a G12D, G12V, G12C, G12S, G12A, or G12R mutation; or a mutation at codon 13, such as a G13D mutation; or a mutation at codon 61, such as a Q61H mutation, or cancers that may benefit from inhibition of wild-type KRAS.

In an aspect, the present disclosure provides compositions comprising compounds represented by Formula X:

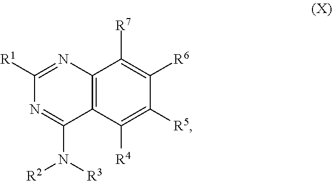

or a salt (e.g., pharmaceutically acceptable salt), ester, tautomer, prodrug, zwitterionic form, or stereoisomer(s) thereof, wherein R¹, R², R³, R⁴, R⁵, R, and R⁷ are as provided herein. In some embodiments, the compound is a compound according to any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IHM1, IIN, IIN1, IIP, IP1, IIQ, IIQ1, HR, IIR1, IIS, IIS1, IIT, IIT1, IIU, IU1, TIV, IIV1, IIW, IIW1, HIX, IX1, IIY, IIY1, IIZ, IIZ1, IIAA, IIAA1, III, III′, III-a, IIA, IIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a provided herein, or a salt (e.g., pharmaceutically acceptable salt), ester, tauntomer, prodrug, zwitterionic form, or stereoisomer(s) thereof. In some embodiments, a compound provided herein, or a salt, ester, tautomer, zwitterionic form, or stereoisomer(s) thereof, can modulate (e.g., inhibit) the activity of a KRAS protein, such as a KRAS protein having a mutation at codon 12, such as a G12D, G12V, G12C, G12S, G12A, or G12R mutation; a KRAS protein having a mutation at codon 13, such as G3D; a KRAS protein having a mutation at codon 61, such as Q61H; or a wild-type KRAS. In some embodiments, a compound provided herein, or a salt, ester, tautomer, zwitterionic form, or stereoisomer(s) thereof, is capable of interacting with a KRAS protein comprising a glycine to aspartic acid, valine, cysteine, serine, alanine, or arginine mutation at codon 12 (i.e., a G12D, G12V, G12C, G12S, G12A, or G12R mutation) or a glycine to aspartic acid mutation at codon 13 (e.g., a G13D mutation) or a glutamine to histidine mutation at codon 61 (e.g., a Q61H mutation) in both its active and inactive conformations. In some embodiments, a compound provided herein, or a salt, ester, tautomer, zwitterionic form, or stereoisomer(s) thereof, has inhibitory activity against wild-type KRAS. In some embodiments, a compound provided herein, or a salt, ester, tautomer, zwitterionic form, or stereoisomer(s) thereof, is capable of binding a KRAS protein in an active (“WP-bound”) conformation. In some embodiments, a compound provided herein, or a salt, ester, tautomer, zwitterionic form, or stereoisomer(s) thereof, is capable of binding a KRAS protein in an inactive (“GDP-bound”) conformation. In some embodiments, a compound provided herein, or a salt, ester, tautomer, zwitterionic form, or stereoisomer(s) thereof, is capable of binding a KRAS protein in both its active (“GTP-bound”) and inactive (“GDP-bound”) conformations.

In another aspect, the present disclosure provides a pharmaceutical composition comprising a compound provided herein (e.g., a compound represented by one of Formulas I, I-a, IA, IA1, IA2, IB, IB1, IB2, IC, IC1, IC2, ID, ID1, ID2, IE, IE1, IE2, IF, IF1, IF2, II, II′, II-a, IA, IA1, IIB, IIB1, IIC, IIC1, IID, ID1, IIE, IIE1, IIF, IIF1, IIG, IIG1, IIH, IIH1, IIJ, IIJ1, IIK, IIK1, HL, IIL1, IIM, IIM1, IIN, IIN1, IIP, IIP1, IIQ, IIQ1, IIR, IIR1, HS, IIS1, IIT, IIT1, IIU, IIU1, IIV, IIV1, IIW, IIW1, IIX, IIX1, HY, IIY1, IIZ, IIZ1, IIAA, IIAA1, III, III′, III-a, IIA, IIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a, or any other formula set forth herein), or a salt, ester, tautomer, prodrug, zwitterionic form, or stereoisomer(s) thereof, together with a pharmaceutically acceptable carrier.

In a further aspect, the present disclosure provides a method of inhibition of KRAS activity in a human or animal subject for the treatment of a disease such as cancer, including pancreatic cancer (e.g., pancreatic ductal adenocarcinoma (PDAC)), colorectal cancer, endometrial endometrioid adenocarcinoma, rectal adenocarcinoma, gastric cancer, and lung cancer, using, e.g., a compound provided herein (e.g., a compound represented by one of Formulas I, I-a, IA, IA1, IA2, IB, IB1, IB2, IC, IC1, IC2, ID, ID1, ID2, IE, IE1, IE2, IF, IF1, IF2, I, II′, II-a, IIA, IIA1, IIB, IIB1, IIC, IIC1, IID, IID1, IIE, IIE1, IIF, IIF1, IIG, IIG1, IIK, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, III, II′, III-a, IIIA, IIIA1, IIIB, IIIC, IIIC1, IIID, IIIE, IIIF, IIIG, IIIH, IIII, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a, or any other formula set forth herein), or a salt, ester, tautomer, prodrug, zwitterionic form, or stereoisomer(s) thereof, or a pharmaceutical composition comprising the same.

In another aspect, the present disclosure provides a use of a compound provided herein (e.g., a compound represented by one of Formulas I, I-a, IA, IA1, IA2, IB, IB1, IB2, IC, IC1, IC2, ID, ID1, ID2, IE, IE1, IE2, IF, IF1, IF2, I, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, ITT, IIT1, IIU, IIU1, IIV, IIV1, IIW, IIW1, IIX, IIX1, HY, IIY1, IIZ, IIZ1, IIAA, IIAA1, III, II′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a, or any other formula set forth herein), or a salt, ester, tautomer, zwittenionic form, or stereoisomer(s) thereof, in the manufacture of a medicament for the treatment of a disease, disorder, or condition (e.g., a cancer) ameliorated, treated, inhibited, or reduced by inhibition of KRAS, including KRAS having a mutation at codon 12 (e.g., a G12D, G12V, G12C, G12S, G12A, or G12R mutation), KRAS having a mutation at codon 13 (e.g., a G13D mutation), KRAS having a mutation at codon 61 (e.g., a Q61H mutation), or a wild-type KRAS. In some embodiments, the disease, disorder, or condition is pancreatic cancer (e.g., pancreatic ductal adenocarcinoma (PDAC)), colorectal cancer, or lung cancer.

In a further aspect, the present disclosure provides a compound as provided herein (e.g., a compound represented by one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, III, III′, III-a, IIA, IIA1, IIIB, IIC, IIIC1, IIID, IIIE, IIIF, IIIG, IIIH, IIIJ, IIIK, IIIL, IIIM, IIIN, HIP, IIIQ, IIIR, IIIS, IIIT, IV, IV-a, IVA, IVB, IVC, V, V-a, VA, VB, VC, VI, and VI-a, or any other formula set forth herein), or a salt, ester, tautomer, zwitterionic form, or stereoisomer(s) thereof, for use as a medicament. In some embodiments, the medicament is used in the treatment of a disease, disorder, or condition (e.g., a cancer). In some embodiments, the disease, disorder, or condition is pancreatic cancer (e.g., pancreatic ductal adenocarcinoma (PDAC)), colorectal cancer, or lung cancer.

### DETAILED DESCRIPTION

The present disclosure provides compounds (e.g., compounds of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IN, IIN1, IIP, IIP1, IIQ, IIQ1, IIR, IIR1, IIS, IIS1, IIT, IIT1, IIU, IIU1, IV, IIV1, IIW, IIW1, IIX, IIX1, IIY, IIY1, IIZ, IIZ1, IIAA, IIAA1, III, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIR, IIIS, IIIT, IV, IV-a, IVA, IVB, IVC, V, V-a, VA, VB, VC, VI, and VI-a), which compounds may possess useful KRAS inhibitory activity, and may be used in the treatment or prophylaxis of a disease, disorder, or condition in which KRAS plays an active role. In particular, certain compounds provided herein may possess useful inhibitory activity of a KRAS protein having a mutation at codon 12 (e.g., a G12D, G12V, G12C, G12S, G12A, or G12R mutation), a KRAS protein having a mutation at codon 13 (e.g., a G13D mutation), a KRAS protein having a mutation at codon 61 (e.g., a Q61H mutation), or a wild-type KRAS protein, which KRAS protein is in an active (GTP-bound) or inactive (GDP-bound) conformation. Certain compounds provided herein may be capable of inhibiting both active and inactive forms of KRAS. The present disclosure also provides pharmaceutical compositions comprising one or more compounds provided herein together with a pharmaceutically acceptable carrier, as well as methods of making and using the compounds and compositions. The present disclosure also provides methods for inhibiting KRAS, including a KRAS protein having a mutation at codon 12 (e.g., a G12D, G12V, G12C, G12S, G12A, or G12R mutation), a KRAS protein having a mutation at codon 13 (e.g., a G13D mutation), a KRAS protein having a mutation at codon 61 (e.g., a Q61H mutation), or a wild-type KRAS protein, which KRAS is in an active or inactive conformation. In an aspect, the present disclosure provides a method for treating a disorder mediated by KRAS including a KRAS protein having a mutation at codon 12 (e.g., a G12D, G12V, G12C, G12S, G12A, or G12R mutation), a KRAS protein having a mutation at codon 13 (e.g., a G13D mutation), a KRAS protein having a mutation at codon 61 (e.g., a Q61H mutation), or a wild-type KRAS protein in a subject in need of such treatment, which method comprises administering to the subject a therapeutically effective amount of a compound or composition provided herein. Also provided herein is the use of certain compounds provided herein in the manufacture of a medicament for the treatment of a disease, disorder, or condition ameliorated, treated, inhibited, or reduced by inhibition of KRAS, including a KRAS protein having a mutation at codon 12 (e.g., a G12D, G12V, G12C, G12S, G12A, or G(12R mutation), a KRAS protein having a mutation at codon 13 (e.g., a G13D mutation), a KRAS protein having a mutation at codon 61 (e.g., a Q61H mutation), or a wild-type KRAS protein. In some embodiments, the disease, disorder, or condition is a cancer (e.g., as described herein).

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

“About,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

“Acyl,” as used herein, alone or in combination, refers to a carbonyl attached to an alkenyl, alkyl, aryl, cycloalkyl, heteroaryl, heterocycle, or any other moiety where the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

“Alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C═C—)]. Examples of suitable alkenyl radicals include ethenyl, propenyl, 2-methylpropenyl, 1,4-butadienyl and the like. Unless otherwise specified, the term “alkenyl” may include “alkenylene” groups.

“Alkynyl” refers to either a straight chain or branched-chain hydrocarbon having at least 2 carbon atoms and at least one triple bond and having the number of carbon atoms indicated (i.e., C₂₋₆ means to two to six carbons). Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, and 1,3,5-hexatriynyl.

“Alkoxy,” as used herein, alone or in combination, refers to an alkyl ether radical, wherein the term alkyl is as described herein. Examples of suitable alkyl ether radicals include methoxy, ethoxy, n-propoxy, isopropoxy, n-butoxy, iso-butoxy, sec-butoxy, tert-butoxy, and the like.

“Alkyl,” as used herein, alone or in combination, refers to a straight-chain or branched-chain alkyl radical containing from 1 to 20 carbon atoms (e.g., C₁₋₂₀ alkyl). In certain embodiments, said alkyl will comprise from 1 to 10 carbon atoms (e.g., C₁₋₁₀ alkyl). In further embodiments, said alkyl will comprise from 1 to 8 carbon atoms (e.g., C₁₋₈ alkyl). In further embodiments, said alkyl will comprise from 1 to 6 carbon atoms (e.g., C₁₋₆ alkyl). In further embodiments, said alkyl will comprise from 1 to 3 carbon atoms (e.g., C₁₋₃ alkyl). Alkyl groups are unsubstituted or substituted as defined herein. Examples of alkyl radicals include methyl, ethyl, n-propyl, isopropyl, n-butyl, isobutyl, sec-butyl, tert-butyl, pentyl, iso-amyl, hexyl, octyl, nonyl, and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

“Alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino, and the like.

“Alkylthio,” as used herein, alone or in combination, refers to an alkyl thioether (R—S—) radical wherein the term alkyl is as described herein and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

“Amido” and “carbamoyl,” as used herein, alone or in combination, refer to an amino group as described herein attached to the parent molecular moiety through a carbonyl group, or vice versa. The “amido” group as used herein incudes “C-amido” and “N-amido” groups. The term “C-amido” as used herein, alone or in combination, refers to a —C(O)N(RR′) group with R and R′ as defined herein or as defined by the specifically enumerated “R” groups designated. In some embodiments, the “amido” group includes —C(O)NH₂, C₁₋₆alkylamido, and di(C₁₋₆alkyl)amido. The term “C₁₋₆alkylamido”, as used herein, refers to —C(O)NH(C₁₋₆alkyl), wherein C₁₋₆alkyl is as defined herein. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

“Amino,” as used herein, alone or in combination, refers to —NRR′, wherein R and R′ are independently selected from hydrogen, alkyl, acyl, heteroalkyl, aryl, cycloalkyl, heteroaryl, and heterocycloalkyl, any of which may themselves be unsubstituted or substituted. Additionally, R and R′ may combine to form a heterocycloalkyl, which is unsubstituted or substituted. An “amino” group may be a primary amine (e.g., —NH₂), secondary or di-substituted amine (e.g., —NHR where R is not hydrogen), or tertiary or tri-substituted amine (e.g., —NRR′ where neither R nor R′ is hydrogen).

“Aryl,” as used herein, alone or in combination, means a carbocyclic aromatic system containing one, two, or three rings wherein such polycyclic ring systems are fused together. The term “aryl” embraces aromatic groups such as phenyl, naphthyl, anthracenyl, and phenanthryl. An aryl moiety may include, for example, between 5 to 20 carbon atoms, such as between 5 to 12 carbon atoms, such as 5 or 6 carbon atoms.

“Arylalkenyl” or “aralkenyl,” as used herein, alone or in combination, refers to an aryl group attached to the parent molecular moiety through an alkenyl group.

“Arylalkoxy” or “aralkoxy,” as used herein, alone or in combination, refers to an aryl group attached to the parent molecular moiety through an alkoxy group.

“Arylalkyl” or “aralkyl,” as used herein, alone or in combination, refers to an aryl group attached to the parent molecular moiety through an alkyl group.

“Aryloxy,” as used herein, alone or in combination, refers to an aryl group attached to the parent molecular moiety through an oxy.

“Carbamate,” as used herein, alone or in combination, refers to an ester of carbamic acid (—NHCOO—) which may be attached to the parent molecular moiety from either the nitrogen or acid end, and which is unsubstituted or substituted as defined herein.

“O-carbamyl” as used herein, alone or in combination, refers to a —OC(O)NRR′ group, with Rand R′ as defined herein.

“N-carbamyl” as used herein, alone or in combination, refers to a ROC(O)NR′— group, with Rand R′ as defined herein.

“Carbonyl,” as used herein, when alone includes formyl [—C(O)H] and in combination is a —C(O)— group.

“Carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

“Cyano,” as used herein, alone or in combination, refers to —CN.

“Cycloalkyl,” or, alternatively, “carbocycle,” as used herein, alone or in combination, refers to a saturated or partially saturated monocyclic, bicyclic, or tricyclic alkyl group wherein each cyclic moiety contains from 3 to 12 carbon atom ring members and which may optionally be a benzo fused ring system which is unsubstituted or substituted as defined herein. A carbocycle may comprise a bridged ring system and/or a spiro ring system (e.g., a system including two rings sharing a single carbon atom). The term “cycloalkenyl” refers to a cycloalkyl group having one or two double bonds. In certain embodiments, said cycloalkyl (or cycloalkenyl) will comprise from 5 to 7 carbon atoms. Examples of such groups include cyclopropyl, cyclobutyl, cyclopentyl, cyclohexyl, cycloheptyl, cyclobutenyl, cyclopentenyl, cyclohexenyl, tetrahydronapthyl, indanyl, octahydronaphthyl, 2,3-dihydro-1H-indenyl, adamantyl, and the like. “Bicyclic” and “tricyclic” as used herein are intended to include both fused ring systems, such as decahydronaphthalene and octahydronaphthalene, as well as the multicyclic (multicentered) saturated or partially unsaturated type. The latter type of isomer is exemplified in general by bicyclo[1,1,1]pentane, camphor, adamantane, and bicyclo[3,2,1]octane.

“Ester,” as used herein, alone or in combination, refers to a carboxy group bridging two moieties linked at carbon atoms.

“Ether,” as used herein, alone or in combination, refers to an oxy group bridging two moieties linked at carbon atoms.

“Halo,” or “halogen.” as used herein, alone or in combination, refers to fluorine, chlorine, bromine, or iodine.

“Haloalkoxy,” as used herein, alone or in combination, refers to a haloalkyl group attached to the parent molecular moiety through an oxygen atom.

“Haloalkyl,” as used herein, alone or in combination, refers to an alkyl radical having the meaning as described herein wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro, or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂-), chloromethylene (—CHCl—) and the like.

“Heteroalkyl,” as used herein, alone or in combination, refers to a stable straight or branched hydrocarbon chain, fully saturated or containing from 1 to 3 degrees of unsaturation, consisting of the stated number of carbon atoms and from one to three heteroatoms selected from N, O, and S, and wherein the N and S atoms may optionally be oxidized and the N heteroatom may optionally be quaternized. The heteroatom(s) may be placed at any interior position of the heteroalkyl group. Up to two heteroatoms may be consecutive, such as, for example, —CH₂—NH—OCH₃.

“Heteroaryl,” as used herein, alone or in combination, refers to a 3- to 15-membered aromatic monocyclic ring, or a fused monocyclic, bicyclic, or tricyclic ring system in which at least one of the fused rings is aromatic, which ring or ring system contains at least one atom selected from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, imidazolyl, pyrazolyl, pyridyl, pyrimidinyl, pyrazinyl, pyridazinyl, triazolyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, phenanthrolinyl, dibenzofuranyl, acridinyl, phenanthridinyl, xanthenyl and the like.

“Heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently selected from nitrogen, oxygen, and sulfur. In some embodiments, a heterocycle comprises a heteroaryl ring fused to a saturated, partially unsaturated, or fully unsaturated (but nonaromatic) ring that optionally contains a heteroatom. In some embodiments, a heterocycle comprises an aryl ring fused to a saturated, partially unsaturated, or fully unsaturated (but nonaromatic) ring that contains a heteroatom. In some embodiments, a heterocycle comprises a carbocycle ring fused to a saturated, partially unsaturated, or fully unsaturated ring that contains a heteroatom. In some embodiments, a heterocycle comprises a first ring that is saturated, partially unsaturated, or fully unsaturated ring that contains a heteroatom and a second ring that is saturated, partially unsaturated, or fully unsaturated ring that optionally contains a heteroatom. In some embodiments, the first ring and the second ring share a single heteroatom. In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from S to 6 ring members in each ring. A heterocycle may comprise a bridged ring system and/or a spiro ring system (e.g., a system including two rings sharing a single atom, such as a single carbon atom). “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, 4,5,6,7-tetrahydropyrazolo[1,5-a]pyrazine, 4,5,6,7-tetrahydro-[1,2,3]triazolo[1,5-a]pyrazine, 4,5,6,7-tetrahydro-1H-pyrazolo[3,4-c]pyridine, 1-methyl-4,5,6,7-tetrahydro-1H-pyrazolo[3,4-c]pyridine, 5,6,7,8-tetrahydro-[1,2,4]triazolo[4,3-a]pyrazine, 5,6,7,8-tetrahydro-[1,2,4]triazolo[1,5-a]pyrazine, 5,6,7,8-tetrahydro-4H-pyrazolo[1,5-a][1,4]diazepine, 3-oxa-9-azabicyclo[3.3.1]nonane, hexahydro-3,6-epiminofuro[3,2-b]furan, and the like. The heterocycle groups are unsubstituted or substituted unless specifically prohibited.

“Hydrazinyl” as used herein, alone or in combination, refers to two amino groups joined by a single bond, i.e., —N—N—.

“Hydroxy,” as used herein, alone or in combination, refers to —OH.

“Hydroxyalkyl,” as used herein, alone or in combination, refers to a hydroxy group attached to the parent molecular moiety through an alkyl group.

“Iminohydroxy,” as used herein, alone or in combination, refers to —N(OH) and —N—O—.

“Lower amino,” as used herein, alone or in combination, refers to —NRR′, wherein R and R′ are independently selected from hydrogen and lower alkyl (e.g., C₁₋₆ alkyl), either of which is unsubstituted or substituted.

“Mercaptyl” as used herein, alone or in combination, refers to an RS— group, where R is as defined herein.

“Nitro,” as used herein, alone or in combination, refers to —NO₂.

“Oxy” or “oxa,” as used herein, alone or in combination, refer to —O—.

“Oxo.” as used herein, alone or in combination, refers to ═O.

“Perhaloalkoxy” refers to an alkoxy group where all of the hydrogen atoms are replaced by halogen atoms.

“Perhaloalkyl” as used herein, alone or in combination, refers to an alkyl group where all of the hydrogen atoms are replaced by halogen atoms.

“Ring,” or equivalently, “cycle,” as used herein, in reference to a chemical structure or portion thereof, means a group in which every atom is a member of a common cyclic structure. A ring can be saturated or unsaturated, including aromatic, unless otherwise provided, and may have between 3 and 9 members. If the ring is a heterocycle, it may contain between 1 and 4 heteroatoms or heteroatom-comprising groups selected from B, N, O, S, C(O), and S(O)ₘ, wherein m is 0, 1, or 2. Unless specifically prohibited, a ring is unsubstituted or substituted. Two or more rings may be fused together (e.g., they may share a bond and two common atoms). Two or more rings may be linked together in a spiro arrangement such that only a single atom is shared between two rings. Two or more rings may also or alternatively be configured in a bridged arrangement such that three or more atoms are shared between two or more rings.

“Sulfonate,” “sulfonic acid,” and “sulfonic,” as used herein, alone or in combination, refer to the —SO₃H group and its anion as the sulfonic acid is used in salt formation.

“Sulfanyl,” as used herein, alone or in combination, refers to-S—.

“Sulfinyl,” as used herein, alone or in combination, refers to —S(O)—.

“Sulfonyl,” as used herein, alone or in combination, refers to —S(O)₂—.

“N-sulfonamido” refers to a RS(═O)₂NR′— group with R and R′ as defined herein.

“S-sulfonamido” refers to a —S(═O)₂NRR′, group, with R and R′ as defined herein.

“Tautomer”, as use herein, alone or in combination, refers to one of two or more isomers that rapidly interconvert. Generally, this interconversion is sufficiently fast so that an individual tautomer is not isolated in the absence of another tautomer. The ratio of the amount of tautomers can be dependent on solvent composition, ionic strength, and pH, as well as other solution parameters. The ratio of the amount of tautomers can be different in a particular solution and in the microenvironment of a biomolecular binding site in said solution. Examples of tautomers that are well known in the art include keto/enol, enamine/imine, and lactam/lactim tautomers. Examples of tautomers that are well known in the art also include 2-hydroxypyridine/2(1H)-pyridone and 2-aminopyridine/2(1H)-iminopyridone tautomers.

“Thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

“Thiol,” as used herein, alone or in combination, refers to an —SH group.

“Thiocarbonyl,” as used herein, when alone includes thioformyl —C(S)H and in combination is a —C(S)— group.

“N-thiocarbamyl” refers to an ROC(S)NR′— group, with Rand R′ as defined herein.

“O-thiocarbamyl” refers to a —OC(S)NRR′ group, with Rand R′ as defined herein.

“Thiocyanato” refers to a —CNS group.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

As described herein, groups may be substituted or unsubstituted (e.g., “optionally substituted”). Unless otherwise specified, any group may be substituted with one or more substituents, such as one or more substituents provided herein. Examples of substituents that may substitute a group include, but are not limited to, one or more substituents independently selected from the following groups or a particular designated set of groups, alone or in combination: alkyl (e.g., C₁₋₂₀ alkyl, such as C₁₋₁₀ alkyl, such as C₁₋₆ alkyl, such as C₁₋₃ alkyl), alkenyl (e.g., C₂₋₂₀ alkenyl, such as C₂₋₁₀ alkenyl, such as C₂₋₄ alkenyl), alkynyl (e.g., C₂₋₂₀ alkynyl, such as C₂₋₁₀ alkynyl, such as C₂₋₆ alkynyl), alkanoyl (e.g., C₁₋₂₀ alkanoyl, such as C₁₋₁₀ alkanoyl, such as C₁₋₁₀ alkanoyl), heteroalkyl (e.g., a heteroalkyl moiety including 1-20 carbon atoms and 1-6 heteroatoms, such as a heteroalkyl moiety including 1-6 carbon atoms and 1-3 heteroatoms), haloalkyl (e.g., a halo-substituted C₁₋₂₀ alkyl, such as a halo-substituted C₁₋₁₀ alkyl, a halo-substituted C₁₋₆ alkyl), haloalkenyl (e.g., a halo-substituted C₂₋₂₀ alkenyl, such as a halo-substituted C₂₋₆ alkenyl), haloalkynyl (e.g., a halo-substituted C₂₋₂₀ alkynyl, such as a halo-substituted C₂₋₆ alkynyl), perhaloalkyl (e.g., C₁₋₂₀ perhaloalkyl, such as C₁₋₆ perhaloalkyl, such as C₁₋₆ perhaloalkyl), perhaloalkoxy (e.g., C₁₋₂₀ perhaloalkoxy, such as C₁₋₆ perhaloalkoxy), phenyl, aryl (e.g., C₅₋₂₀ aryl, such as C₅₋₁₀ aryl, such as C₁₋₆ aryl), aryloxy (e.g., C₁₋₂₀ aryloxy, such as C₅₋₆ aryloxy, such as C₅₋₆ aryloxy), alkoxy (e.g., C₁₋₂₀ alkoxy, such as C₁₋₁₀ alkoxy, such as C₁₋₆ alkoxy), haloalkoxy (e.g., C₁₋₂₀ haloalkoxy, such as C₁₋₁₀ haloalkoxy, such as C₁₋₆ haloalkoxy), oxo, acyloxy (e.g., an acyloxy group including 1-20 carbon atoms, such as 1-10 carbon atoms, such as 1-6 carbon atoms), carbonyl (e.g., C(O) or C═O), carboxyl (e.g., C(O)O), alkylcarbonyl (e.g., C₁₋₂₀ alkylcarbonyl, such as C₁₋₁₀ alkylcarbonyl, such as C₁₋₆ alkylcarbonyl, such as C₁₋₆, alkylcarbonyl), carboxyester (e.g., C(O)OR where R is, e.g., alkyl (e.g., C₁₋₂₀ alkyl, such as C₁₋₁₀ alkyl, such as C₁₋₆ alkyl, such as C₁₋₃ alkyl), alkenyl (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), carboxamido, cyano (e.g., CN), hydrogen, halogen (e.g., iodine, bromine, chlorine, or fluorine), hydroxy, amino (e.g., NR′R″ where R′ and R″ are independently, e.g., hydrogen, alkyl (e.g., C₁₋₂₀ alkyl, such as C₁₋₁₀ alkyl, such as C₁₋₆ alkyl, such as C₁₋₃ alkyl), alkenyl (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), alkylamino (e.g., NR′R″ where R′ is alkyl (e.g., C₁₋₂₀ alkyl, such as C₁₋₁₀ alkyl, such as C₁₋₆ alkyl, such as C₁₋₃ alkyl) and R′ is, e.g., hydrogen, alkyl (e.g., C₁₋₂ alkyl, such as C₁₋₁₀ alkyl, such as C₁₋₆ alkyl, such as C₁₋₃ alkyl), alkenyl (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), arylamino (e.g., NR′R″ where R′ is aryl (e.g., C₅₋₂₀ aryl, such as C₅₋₁₀ aryl, such as C₅₋₆ aryl) and R″ is, e.g., hydrogen, alkyl (e.g., C₁₋₂₀ alkyl, such as C₁₋₁₀ alkyl, such as C₁₋₆ alkyl, such as C₁₋₃ alkyl), alkenyl (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), amido (e.g., C(O)NR′R″ where R′ and R″ are independently, e.g., hydrogen, alkyl (e.g., C₁₋₂₀ alkyl, such as C₁₋₁₀ alkyl, such as C₁₋₆ alkyl, such as C₁₋₃ alkyl), alkenyl (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), nitro (e.g., NO₂), thiol (e.g., SH), alkylthio (e.g., C₁₋₂₀ alkyl substituted with a thiol group, such as C₁₋₁₀ alkyl substituted with a thiol group, such as C₁₋₆ alkyl substituted with a thiol group, such as C₁₋₃ alkyl substituted with a thiol group), haloalkylthio (e.g., C₁₋₂₀ haloalkylthio, such as C₁₋₁₀ haloalkylthio, such as C₁₋₆ haloalkylthio, such as C₁₋₃ haloalkylthio), perhaloalkylthio (e.g., C₁₋₂₀ perhaloalkylthio, such as C₁₋₁₀ perhaloalkylthio, such as C₁₋₆ perhaloalkylthio, such as C₁₋₃ perhaloalkylthio), arylthiol (e.g., C₅₋₂₀ arylthiol, such as C₅₋₁₀ arylthiol, such as C₅₋₆ arylthiol), sulfonate (e.g., S(O)₂OR where R is, e.g., alkyl (e.g., C₁₋₂₀ alkyl, such as C₁₋₁₀ alkyl, such as C₁₋₆ alkyl, such as C₁₋₃, alkyl), alkenyl (e.g., C₂₋₂₀ alknyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), sulfonic acid (e.g., S(O)₂OH), trisubstituted silyl (e.g., SiR′R″R* where R′, R″, and R* are independently selected from, e.g., alkyl (e.g., C₁₋₂₀ alkyl, such as C₁₋₁₀ alkyl, such as C₁₋₆ alkyl, such as C₁₋₃ alkyl), alkenyl (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein; in some cases, a trisubstituted silyl can be trimethylsilyl), N₃, SCH₃, C(O)CH₃, CO₂CH₃, CO₂H, pyridinyl, thiophene, furanyl, carbamate, and urea.

Additional groups may also be contemplated. Where structurally feasible, two substituents may be joined together to form a fused five-, six-, or seven-membered carbocyclic or heterocyclic ring consisting of zero to three heteroatoms (e.g., N, O, S, etc.), for example forming methylenedioxy or ethylenedioxy. An unsubstituted or substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “unsubstituted or substituted with.”

The terms R, R′, R″, R*, etc., appearing by themselves and without a number designation, unless otherwise defined, refer to a moiety selected from hydrogen, alkyl, cycloalkyl, heteroalkyl, aryl, heteroaryl and heterocycloalkyl, any of which is unsubstituted or substituted (e.g., as described herein). Such R and R′ groups should be understood to be unsubstituted or substituted as defined herein. Whether an R group has a number designation or not, every R group, including R, R′ and RV where n=(1, 2, 3, . . . n), every substituent, and every term should be understood to be independent of every other in terms of selection from a group. Should any variable, substituent, or term (e.g., aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(O)N(R)— may be attached to the parent moiety at either the carbon or the nitrogen.

“Bond” refers to a covalent linkage between two atoms, or two moieties when the atoms joined by the bond are considered to be part of larger substructure. A bond may be single, double, or triple unless otherwise specified. A dashed link between two atoms in a drawing of a molecule indicates that an additional bond may be present or absent at that position.

Asymmetric centers may exist in the compounds disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, atropisomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may cist as geometric isomers. The present disclosure includes all cis, trans, syn, anti, entgegen (E), and zusammen (Z) isomers as well as the appropriate mixtures thereof. Additionally, compounds may exist as tautomers; all tautomeric isomers are provided by this disclosure. Additionally, the compounds provided herein may comprise conformational isomers, which compounds comprise groups that can orient in different conformations in relation to another moiety. Additionally, the compounds disclosed herein can exist in unsolvated as well as solvated forms with pharmaceutically acceptable solvents such as water, ethanol, and the like. In general, the solvated forms are considered equivalent to the unsolvated forms.

“Combination therapy” means the administration of two or more therapeutic agents to treat a therapeutic condition or disorder described in the present disclosure. Such administration encompasses co-administration of these therapeutic agents in a substantially simultaneous manner, such as in a single dose unit (e.g., capsule) having a fixed ratio of active ingredients or in multiple, separate dose units (e.g., capsules) for each active ingredient. In addition, such administration also encompasses use of each type of therapeutic agent in a sequential manner. In either case, the treatment regimen will provide beneficial effects of the drug combination in treating the conditions or disorders described herein.

“KRAS inhibitor” is used herein to refer to a compound that exhibits an ICs with respect to KRAS activity of no more than about 100 μM and more typically not more than about 50 μM, as measured in the assays described generally herein, such as a surface plasmon resonance KRAS-G12D, G12V, G12C, G12S, G12A, G12R, G3D, or Q61H mutation or wild-type KRAS protein binding assay; and/or a KRAS G12D, G12V, G12C, G12S, G12A, G12R, G13D, or Q61H mutation or wild-type KRAS protein-effector protein interaction disruption assay. “IC₅₀” is that concentration of inhibitor which reduces the activity of an enzyme (e.g., KRAS) to half-maximal level. Certain compounds disclosed herein have been discovered to exhibit inhibition against KRAS. In certain embodiments, compounds exhibit an ICs with respect to KRAS (e.g., a KRAS protein having a mutation at codon 12 (e.g., a G12D, G12V, G12C, G12S, G12A, or G12R mutation), a KRAS protein having a mutation at codon 13 (e.g., a G13D mutation), a KRAS protein having a mutation at codon 61 (e.g., a Q61H mutation), or a wild-type KRAS protein) of no more than about 50 μM; in further embodiments, compounds exhibit an IC₅₀ with respect to KRAS (e.g., a KRAS protein having a mutation at codon 12 (e.g., a G12D, G12V, G12C, G12S, G12A, or G12R mutation), a KRAS protein having a mutation at codon 13 (e.g., a G13D mutation), a KRAS protein having a mutation at codon 61 (e.g., a Q61H mutation), or a wild-type KRAS protein) of no more than about 10 μM; in yet further embodiments, compounds exhibit an IC₅₀ with respect to KRAS (e.g., a KRAS protein having a mutation at codon 12 (e.g., a G12D, G12V, G12C, G12S, G12A, or G12R mutation), a KRAS protein having a mutation at codon 13 (e.g., a G13D mutation), a KRAS protein having a mutation at codon 61 (e.g., a Q61H mutation), or a wild-type KRAS protein) of not more than about 1 μM; in yet further embodiments, compounds exhibit an IC₅₀ with respect to KRAS (e.g., a KRAS protein having a mutation at codon 12 (e.g., a G12D, G12V, G12C, G12S, G12A, or G12R mutation), a KRAS protein having a mutation at codon 13 (e.g., a G13D mutation), a KRAS protein having a mutation at codon 61 (e.g., a Q61H mutation), or a wild-type KRAS protein) of not more than about 200 nanomolar (nM), as measured in the KRAS assay described herein. In some embodiments, compounds exhibit an IC₅₀ with respect to KRAS (e.g., a KRAS protein having a mutation at codon 12 (e.g., a G12D, G12V, G12C, G12S, G12A, or G12R mutation), a KRAS protein having a mutation at codon 13 (e.g., a G13D mutation), a KRAS protein having a mutation at codon 61 (e.g., a Q61H mutation), or a wild-type KRAS protein) of less than about 50 μM, such as less than about 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less. In certain embodiments, compounds exhibit an IC₅₀ with respect to KRAS (e.g., a KRAS protein having a mutation at codon 12 (e.g., a G12D, G12V, G12C, G12S, G12A, or G12R mutation), a KRAS protein having a mutation at codon 13 (e.g., a G13D mutation), a KRAS protein having a mutation at codon 61 (e.g., a Q61H mutation), or a wild-type KRAS protein) of less than about 1 μM, such as less than about 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less. In some embodiments, compounds exhibit an IC₅₀ with respect to KRAS having a G12D mutation of less than about 50 μM, such as less than about 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less. In some embodiments, compounds exhibit an IC₅₀ with respect to KRAS having a G12V mutation of less than about 50 μM, such as less than about 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less. In some embodiments, compounds exhibit an IC₅₀ with respect to KRAS having a G12R mutation of less than about 50 μM, such as less than about 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less. In some embodiments, compounds exhibit an ICs with respect to KRAS having a G12A mutation of less than about 50 μM, such as less than about 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less. In some embodiments, compounds exhibit an IC₅₀ with respect to KRAS having a G12S mutation of less than about 50 μM, such as less than about 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less. In some embodiments, compounds exhibit an IC₅₀ with respect to KRAS having a G12C mutation of less than about 50 μM, such as less than about 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less. In some embodiments, compounds exhibit an ICs with respect to KRAS having a G13D mutation of less than about 50 μM, such as less than about 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less. In some embodiments, compounds exhibit an IC₅₀ with respect to KRAS having a Q61H mutation of less than about 50 μM, such as less than about 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less. In some embodiments, compounds exhibit an IC₅₀ with respect to wild-type KRAS of less than about 50 μM, such as less than about 40 μM, 30 μM, 20 μM, 10 μM, 9 μM, 8 μM, 7 μM, 6M, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less.

In some embodiments, a KRAS inhibitor has inhibitory activity against KRAS having a G12D mutation that exceeds its inhibitory activity against KRAS having another mutation, such as a Q61H, G12C, G12R, G12S, G12A, G12V, or G13D mutation. For example, in some embodiments, a KRAS inhibitor provided herein has at least two-fold, five-fold, ten-fold, twenty-fold, thirty-fold, forty-fold, fifty-fold, one hundred-fold, or higher inhibitory activity against KRAS having a G12D mutation relative to KRAS having another mutation such as a Q61H, G12C, G12R, G12S, G12A, G12V, or G13D mutation.

In some embodiments, a KRAS inhibitor has inhibitory activity against KRAS having a G12V mutation that exceeds its inhibitory activity against KRAS having another mutation, such as a Q61H, G12C, G12R, G12S, G12A, G12D, or G13D mutation. For example, in some embodiments, a KRAS inhibitor provided herein has at least two-fold, five-fold, ten-fold, twenty-fold, thirty-fold, forty-fold, fifty-fold, one hundred-fold, or higher inhibitory activity against KRAS having a G12V mutation relative to KRAS having another mutation such as a Q61H, G12C, G12R, G12S, G12A, G12D, or G13D mutation.

In some embodiments, a KRAS inhibitor has inhibitory activity against KRAS having a G12R mutation that exceeds its inhibitory activity against KRAS having another mutation, such as a Q61H, G12C, G12D, G12S, G12A, G12V, or G13D mutation. For example, in some embodiments, a KRAS inhibitor provided herein has at least two-fold, five-fold, ten-fold, twenty-fold, thirty-fold, forty-fold, fifty-fold, one hundred-fold, or higher inhibitory activity against KRAS having a G12R mutation relative to KRAS having another mutation such as a Q61H, G12C, G12D, G12S, G12A, G12V, or G13D mutation.

In some embodiments, a KRAS inhibitor has inhibitory activity against KRAS having a G12C mutation that exceeds its inhibitory activity against KRAS having another mutation, such as a Q61H, G12R, G12D, G12S, G12A, G12V, or G13D mutation. For example, in some embodiments, a KRAS inhibitor provided herein has at least two-fold, five-fold, ten-fold, twenty-fold, thirty-fold, forty-fold, fifty-fold, one hundred-fold, or higher inhibitory activity against KRAS having a G12C mutation relative to KRAS having another mutation such as a Q61H, G12R, G12D, G12S, G12A, G12V, or G13D mutation.

In some embodiments, a KRAS inhibitor has inhibitory activity against KRAS having a G12S mutation that exceeds its inhibitory activity against KRAS having another mutation, such as a Q61H, G12C, G12D, G12R, G12A, G12V, or G13D mutation. For example, in some embodiments, a KRAS inhibitor provided herein has at least two-fold, five-fold, ten-fold, twenty-fold, thirty-fold, forty-fold, fifty-fold, one hundred-fold, or higher inhibitory activity against KRAS having a G12S mutation relative to KRAS having another mutation such as a Q61H, G12C, G12D, G12R, G12A, G12V, or G13D mutation.

In some embodiments, a KRAS inhibitor has inhibitory activity against KRAS having a G12A mutation that exceeds its inhibitory activity against KRAS having another mutation, such as a Q61H, G12C, G12D, G12S, G12R, G12V, or G13D mutation. For example, in some embodiments, a KRAS inhibitor provided herein has at least two-fold, five-fold, ten-fold, twenty-fold, thirty-fold, forty-fold, fifty-fold, one hundred-fold, or higher inhibitory activity against KRAS having a G12A mutation relative to KRAS having another mutation such as a Q61H, G12C, G12D, G12S, G12R, G12V, or G13D mutation.

In some embodiments, a KRAS inhibitor has inhibitory activity against KRAS having a G13D mutation that exceeds its inhibitory activity against KRAS having another mutation, such as a Q61H, G12C, G12D, G12S, G12R, G12V, or G12A mutation. For example, in some embodiments, a KRAS inhibitor provided herein has at least two-fold, five-fold, ten-fold, twenty-fold, thirty-fold, forty-fold, fifty-fold, one hundred-fold, or higher inhibitory activity against KRAS having a G13D mutation relative to KRAS having another mutation such as a Q61H, G12C, G12D, G12S, G12R, G12V, or G12A mutation.

In some embodiments, a KRAS inhibitor has inhibitory activity against KRAS having a Q61H mutation that exceeds its inhibitory activity against KRAS having another mutation, such as a G13D, G12C, G12D, G12S, G12R, G12V, or G12A mutation. For example, in some embodiments, a KRAS inhibitor provided herein has at least two-fold, five-fold, ten-fold, twenty-fold, thirty-fold, forty-fold, fifty-fold, one hundred-fold, or higher inhibitory activity against KRAS having a Q61H mutation relative to KRAS having another mutation such as a G3D, G12C, G12D, G12S, G12R, G12V, or G12A mutation.

In some embodiments, a KRAS inhibitor has inhibitory activity against a wild-type KRAS that exceeds its inhibitory activity against KRAS having a Q61H, G13D, G12C, G12D, G12S, G12R, G12V, or G12A mutation. For example, in some embodiments, a KRAS inhibitor provided herein has at least two-fold, five-fold, ten-fold, twenty-fold, thirty-fold, forty-fold, fifty-fold, one hundred-fold, or higher inhibitory activity against a wild-type KRAS relative to KRAS having a Q61H, G13D, G12C, G12D, G12S, G12R, G12V, or G12A mutation.

In some embodiments, a KRAS inhibitor provided herein has greater inhibitory activity against KRAS having a Q61H, G13D, G12D, G12S, G12R, G12V, or G12A mutation or a wild-type KRAS than against KRAS having a G12C mutation. In some embodiments, a KRAS inhibitor provided herein has greater inhibitory activity against KRAS having a G12D, G12V, or G12R mutation than against KRAS having a G12C mutation.

In some embodiments, a KRAS inhibitor provided herein has greater inhibitory activity against KRAS having a G12D mutation than against KRAS having a G12C mutation. In some embodiments, a KRAS inhibitor provided herein has greater inhibitory activity against KRAS having a G12D mutation than against KRAS having a G12R mutation. In some embodiments, a KRAS inhibitor provided herein has greater inhibitory activity against KRAS having a G12D mutation than against a KRAS having a G12S mutation. In some embodiments, a KRAS inhibitor provided herein has greater inhibitory activity against KRAS having a G12D mutation than against KRAS having a G12A mutation. In some embodiments, a KRAS inhibitor provided herein has greater inhibitory activity against KRAS having a G12D mutation than against KRAS having a G12V mutation. In some embodiments, a KRAS inhibitor provided herein has greater inhibitory activity against KRAS having a G12D mutation than against KRAS having a G13D mutation. In some embodiments, a KRAS inhibitor provided herein has greater inhibitory activity against KRAS having a G12D mutation than against KRAS having a Q61H mutation.

In some embodiments, a KRAS inhibitor provided herein has greater inhibitory activity against KRAS having a G12V mutation than against KRAS having a G12C mutation. In some embodiments, a KRAS inhibitor provided herein has greater inhibitory activity against KRAS having a G12V mutation than against KRAS having a G12R mutation. In some embodiments, a KRAS inhibitor provided herein has greater inhibitory activity against KRAS having a G12V mutation than against a KRAS having a G12S mutation. In some embodiments, a KRAS inhibitor provided herein has greater inhibitory activity against KRAS having a G12V mutation than against KRAS having a G12A mutation. In some embodiments, a KRAS inhibitor provided herein has greater inhibitory activity against KRAS having a G12V mutation than against KRAS having a G12D mutation. In some embodiments, a KRAS inhibitor provided herein has greater inhibitory activity against KRAS having a G12V mutation than against KRAS having a G13D mutation. In some embodiments, a KRAS inhibitor provided herein has greater inhibitory activity against KRAS having a G12V mutation than against KRAS having a Q61H mutation.

In some embodiments, a KRAS inhibitor provided herein has greater inhibitory activity against KRAS having a G12R mutation than against KRAS having a G12C mutation. In some embodiments, a KRAS inhibitor provided herein has greater inhibitory activity against KRAS having a G12R mutation than against KRAS having a G12D mutation. In some embodiments, a KRAS inhibitor provided herein has greater inhibitory activity against KRAS having a G12R mutation than against a KRAS having a G12S mutation. In some embodiments, a KRAS inhibitor provided herein has greater inhibitory activity against KRAS having a G12R mutation than against KRAS having a G12A mutation. In some embodiments, a KRAS inhibitor provided herein has greater inhibitory activity against KRAS having a G12R mutation than against KRAS having a G12V mutation. In some embodiments, a KRAS inhibitor provided herein has greater inhibitory activity against KRAS having a G12R mutation than against KRAS having a G13D mutation. In some embodiments, a KRAS inhibitor provided herein has greater inhibitory activity against KRAS having a G12R mutation than against KRAS having a Q61H mutation.

In some embodiments, a KRAS inhibitor provided herein has greater inhibitory activity against active (“GTP-bound”) KRAS having a Q61H, G12D, G12V, G12R, G12A, G12S, G12C, or G13D mutation, or wild-type KRAS, than against an inactive (“GDP-bound”) KRAS having a Q61H, G12D, G12V, G12R, G12A, G12S, G12C, or G13D mutation or wild-type KRAS. In some embodiments, a KRAS inhibitor provided herein has lower inhibitory activity against active (“GTP-bound”) KRAS having a Q61H, G12D, G12V, G12R, G12A, G12S, G12C, or G13D mutation, or wild-type KRAS, than against an inactive (“GDP-bound”) KRAS having a Q61H, G12D, G12V, G12R, G12A, G12S, G12C, or G13D mutation or wild-type KRAS. In some embodiments, a KRAS inhibitor provided herein has inhibitory activity against both active (“GTP-bound”) and inactive (“GDP-bound”) KRAS having a Q61H, G12D, G12V, G12R, G12A, G12S, G12C, or G13D mutation or wild-type KRAS. In some embodiments, a KRAS inhibitor provided herein has similar inhibitory activity against active (“GTP-bound”) and inactive (“GDP-bound”) KRAS having a Q61H, G12D, G12V, G12R, G12A, G12S, G12C, or G13D mutation or wild-type KRAS.

In some embodiments, a KRAS inhibitor provided herein has inhibitory activity against a K-RAS4a splice variant. In some embodiments, a KRAS inhibitor provided herein has inhibitory activity against a K-RAS4b splice variant. In some embodiments, a KRAS inhibitor provided herein has inhibitory activity against both K-RAS4a and K-RAS4b splice variants.

“Therapeutically effective amount” refers to an amount of a compound or of a pharmaceutical composition useful for treating or ameliorating an identified disease, disorder, or condition, or for exhibiting a detectable therapeutic or inhibitory effect. The exact amounts will depend on the purpose of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

“Treat,” “treating,” and “treatment” refer to any indicia of success in the treatment or amelioration of an injury, pathology, disease, disorder, or condition, including any objective or subjective parameter such as abatement; remission; diminishing of symptoms or making the injury, pathology, disease, disorder, or condition more tolerable to the patient; slowing in the rate of degeneration or decline; making the final point of degeneration less debilitating; and/or improving a patient's physical or mental well-being. The treatment or amelioration of symptoms can be based on objective or subjective parameters, including the results of a physical examination, neuropsychiatric exams, and/or a psychiatric evaluation. Treatment may also be preemptive in nature; i.e., it may include prevention of a disease, disorder, or condition, prevention of onset of one or more symptoms of a disease, disorder, or condition, and/or prevention of escalation of a disease, disorder, or condition. Prevention of a disease, disorder, or condition may involve complete protection from disease, and/or prevention of disease progression (e.g., to a later stage of the disease, disorder, or condition). For example, prevention of a disease may not mean complete foreclosure of any effect related to the diseases at any level, but instead may mean prevention of the symptoms of a discase, disorder, or condition to a clinically significant or detectable level.

“Patient” or “subject” refers to a living organism suffering from or prone to a disease, disorder, or condition that can be treated by administration of a compound or pharmaceutical composition as provided herein. Non-limiting examples include humans, rats, mice, rabbits, hamsters, guinea pigs, cats, dogs, non-human primates (e.g., monkeys), goats, pigs, sheep, cows, deer, horses, and other non-mammalian animals. Examples of mammals that can be treated by administration of a compound or pharmaceutical composition provided herein include, for example, rodents (e.g., rats, mice, squirrels, guinea pigs, hamsters, etc.), lagomorphs (e.g., rabbits, hares, etc.), primates (e.g., monkeys, apes, etc.), bovines (e.g., cattle), odd-toed ungulates (e.g., horses), even-toed ungulates (e.g., bovines such as cattle, ovine such as sheep, caprine such as goats, porcine such as pigs, etc.), and marsupials (e.g., kangaroo, wallaby, wallaroo, sugar glider, etc.). In some embodiments, the patient or subject is human. In some embodiments, the patient or subject is a companion animal such as a cat or dog. In some embodiments, the patient or subject is a farm animal such as a goat, sheep, cow, pig, or horse. In some embodiments, the patient or subject is an exotic animal such as a primate (e.g., monkey), marsupial (e.g., kangaroo, wallaby, wallaroo, sugar glider, etc.), or a non-domesticated or hybrid cat or dog.

“Composition,” as used herein, is intended to encompass a product comprising the specified ingredients in the specified amounts, as well as any product, which results, directly or indirectly, from combination of the specified ingredients in the specified amounts. By “pharmaceutically acceptable” it is meant the carrier, diluent, or excipient must be compatible with the other ingredients of the formulation and not deleterious to the recipient thereof.

“Pharmaceutically acceptable excipient” refers to a substance that aids the administration of an active agent to and absorption by a subject. Pharmaceutical excipients useful in the present disclosure include, but are not limited to, binders, fillers, disintegrants, lubricants, coatings, sweeteners, flavors, and colors. One of skill in the art will recognize that other pharmaceutical excipients are useful in the present disclosure.

The term “prodrug” refers to a compound that is made more active in vivo. Certain compounds disclosed herein may also exist as prodrugs. Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent.

Prodrugs are often useful because, in some situations, they may be casica to administer than the compound, or parent drug. They may, for instance, be bioavailable by oral administration whereas the parent drug is not. The prodrug may also have improved solubility in pharmaceutical compositions over the parent drug.

The compounds disclosed herein can exist as therapeutically acceptable salts (also referred to herein as “pharmaceutically acceptable salts”). The present disclosure includes compounds provided herein in the form of salts, including acid addition salts. Suitable salts include those formed with both organic and inorganic acids. Such acid addition salts will normally be pharmaceutically acceptable. However, non-pharmaceutically acceptable salts may be of utility in the preparation and purification of the compound in question. Basic addition salts may also be formed and be pharmaceutically acceptably.

The terms “therapeutically acceptable salt” and “pharmaceutically acceptable salt” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetrmethylammonium, methylamine, dimethylamine, trimethylamine, triethylamine, diethylamine, ethylamine, tributylamine, pyridine, NN-dimethylaniline, N-methylpiperidine, N-methylmorpholine, dicyclohexylamine, procaine, dibenzylamine, N,N-dibenzylphenethylamine, 1-ephenamine, and NN-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

“KRAS positive cancer” refers to a cancer characterized by a KRAS mutation, such as a KRAS Q61, G12C, G12D, G12R, G12A, G12S, G12V, or G13D mutation, and/or by amplified wild-type KRAS activity. In some embodiments, “KRAS positive cancer” refers to a cancer that may benefit from inhibition of KRAS, such as wild-type KRAS or KRAS having a Q61H, G12C, G12D, G12R, G12A, G12S, G12V, or G13D mutation.

“KRAS G12C-positive cancer” refers to a cancer characterized by a KRAS G12C mutation.

“KRAS G12D-positive cancer” refers to a cancer characterized by a KRAS G12D mutation.

“KRAS G12R-positive cancer” refers to a cancer characterized by a KRAS G12R mutation.

“KRAS G12V-positive cancer” refers to a cancer characterized by a KRAS G12V mutation.

“KRAS G12A-positive cancer” refers to a cancer characterized by a KRAS G12A mutation.

“KRAS G12S-positive cancer” refers to a cancer characterized by a KRAS G12S mutation.

“KRAS G13D-positive cancer” refers to a cancer characterized by a KRAS G13D mutation.

“KRAS Q61H-positive cancer” refers to a cancer characterized by a KRAS Q61H mutation.

“Jointly therapeutically effective amount” as used herein means the amount at which the therapeutic agents, when given separately (in a chronologically staggered manner, especially a sequence-specific manner) to a warm-blooded animal, especially to a human to be treated, show an (additive, but preferably synergistic) interaction (joint therapeutic effect). Whether this is the case can be determined inter alia by following the blood levels, showing that both compounds are present in the blood of the human to be treated at least during certain time intervals.

“Synergistic effect” as used herein refers to an effect of at least two therapeutic agents: a KRAS inhibitor, as defined herein, and an additional agent, which additional agent may be an agent configured to treat a disease, disorder, or condition or a symptom thereof. The effect can be, for example, slowing the symptomatic progression of a proliferative discase, such as cancer, particularly lung cancer, or symptoms thereof. Analogously, a “synergistically effective amount” refers to the amount needed to obtain a synergistic effect.

“A,” “an,” or “a(n)”, when used in reference to a group of substituents or “substituent group” herein, mean at least one. For example, where a compound is substituted with “an” alkyl or aryl, the compound is unsubstituted or substituted with at least one alkyl and/or at least one aryl, wherein each alkyl and/or aryl is optionally different. In another example, where a compound is substituted with “a” substituent group, the compound is substituted with at least one substituent group, wherein each substituent group is optionally different.

### Compounds

In an aspect, the present disclosure provides a compound represented by Formula I:

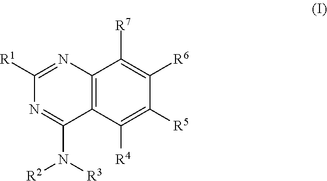

or a salt (e.g., pharmaceutically acceptable salt), ester, tautomer, prodrug, zwitterionic form, or stereoisomer(s) thereof, wherein:
   R¹ is selected from —OR⁸ and

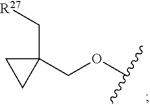

R² is selected from H and C₁₋₆alkyl, wherein any C₁₋₆ alkyl is unsubstituted or is substituted with one or more R¹³;
R³ is selected from a 3-11 membered carbocycle that is unsubstituted or substituted with one or more R¹⁰;
R⁴ is H;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁶ is a bicyclic heteroaryl substituted with one or more R¹⁵;
R⁷ is selected from halogen, —CN, and H;
R⁸ is selected from a heterocycle and an alkylheterocycle, wherein any heterocycle comprises 4-8 members and is unsubstituted or is substituted with one or more R^{a} or R^{b}, and wherein an alkyl moiety ofany alkylheterocycle is selected from C₁₋₆alkyl;
each R¹⁰ is independently selected from —OR¹², —C(O)(C₁₋₆akylene)CN, —C(O)(C₁₋₆alkylene)OH, —C(O)(C₁₋₆alkyl), —C(O)N(R¹⁴)₂, —S(O)₂(C₁₋₆alkyl), halogen, and C₁₋₆alkyl, wherein any C₁₋₆ alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹³;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen; and
R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.

In some embodiments, the present disclosure provides a compound of Formula I, or a salt (e.g., a pharmaceutically acceptable salt) thereof.

In some embodiments, the present disclosure provides a compound of Formula I, wherein:
R¹ is selected from —OR⁸ and

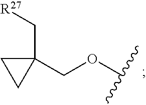

R² is selected from H and C₁₋₆alkyl;
R³ is selected from a 3-11 membered carbocycle that is unsubstituted or substituted with one or more R¹⁰;
R⁴ is H;
R⁵ is selected from H, halogen, —CN, —OR¹², a 5-6 membered heteroaryl, and C₁₋₆alkyl, wherein any C₁₋₆alkyl is unsubstituted or substituted with one or more R¹³;
R⁶ is a bicyclic heteroaryl substituted with one or more R¹⁵;
R⁷ is selected from halogen, —CN, and H;
R⁸ is an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹⁰ is independently selected from —OR¹², —C(O)N(R¹²)₂, halogen, and C₁₋₆alkyl, wherein any C₁₋₆alkyl is unsubstituted or substituted with one or more R¹³;
each R¹² is independently selected from C₁₋₆alkyl and H, wherein any C₁₋₆alkyl is unsubstituted or substituted with one or more R¹³;
each R¹³ is independently selected from halogen;
each R¹⁴ is independently selected from C₁₋₆ alkyl;
each R¹⁵ is independently selected from halogen, —N(R¹²)₂, and —CN;
each R²⁰ is independently selected from —OH, —OC₁₋₆alkyl, and —CN;
R²⁷ is a 3-6 membered heterocycle including one or more heteroatoms selected from N, O, and S, wherein the heterocycle is unsubstituted or substituted with one or more R²⁸;
each R²⁸ is independently selected from halogen; and
R^{a} and R^{b} are each independently selected from halogen, C₁₋₆ alkyl, —OR¹², and H, wherein any C₁₋₆alkyl is unsubstituted.

In some embodiments, the present disclosure provides a compound of Formula I, wherein the compound is of Formula I-a:

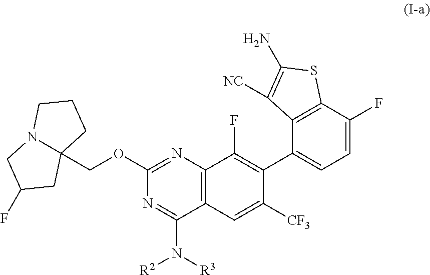

or a salt (e.g., pharmaceutically acceptable salt), ester, tautomer, prodrug, zwitterionic form, or stereoisomer(s) thereof, wherein R² and R³ are as defined for Formula I above and described in classes and subclasses herein, both singly and in combination. In some embodiments, the present disclosure provides a compound of Formula I-a, or a salt (e.g., pharmaceutically acceptable salt) thereof.

In some embodiments for a compound according to Formula I or I-a, R³ is selected from a 3-11 membered carbocycle that is unsubstituted. In some embodiments, R³ is selected from a 3-11 membered carbocycle that is substituted with one or more R¹⁰. In some embodiments, R³ is selected from a 3-8 membered carbocycle that is unsubstituted. In some embodiments, R³ is selected from a 3-8 membered carbocycle that is substituted with one or more R¹⁰. In some embodiments, R³ is a 3-6 membered carbocycle that is unsubstituted or is substituted with one or more R¹⁰. In some embodiments, R³ is a 3-6 membered carbocycle that is unsubstituted. In some embodiments, R³ is a 3-6 membered carbocycle that is substituted with one or more R¹⁰. In some embodiments, R³ is a 3-6 membered carbocycle that is substituted with 1-4 R¹⁰. In some embodiments, R³ is a cyclopropane that is substituted with 0-4 R¹⁰. In some embodiments, R³ is a cyclopropane that is substituted with 1-4 R¹⁰. In some embodiments, R³ is a cyclopropyl that is substituted with one or more R¹⁰, wherein at least one of the one or more R¹⁰ is a C₁₋₆alkyl that is unsubstituted or substituted with one or more R²⁰. In some embodiments, R³ is a cyclobutane that is substituted with 0-4 R¹⁰. In some embodiments, R³ is a cyclobutane that is substituted with 1-4 R¹⁰. In some embodiments, R³ is a cyclopentane that is substituted with 0-4 R¹⁰. In some embodiments, R³ is a cyclopentane that is substituted with 1-4 R¹⁰. In some embodiments, R³ is a cyclohexane that is substituted with 0-4 R″. In some embodiments, R³ is a cyclohexane that is substituted with 1-4 R¹⁰. In some embodiments, R³ is a 3-6 membered carbocycle (e.g., a cyclopropane, cyclobutane, cyclopentane, or cyclohexane) that is substituted with one or more R¹⁰, wherein at least one R¹⁰ is selected from —OR² and a C₁₋₆alkyl substituted with —OH. In some embodiments, R³ is a 3-6 membered carbocycle (e.g., a cyclopropane, cyclobutane, cyclopentane, or cyclohexane) that is substituted with one or more R¹⁰, wherein at least one R¹⁰ is —OR². In some embodiments, R³ is a 3-6 membered carbocycle (e.g., a cyclopropane, cyclobutane, cyclopentane, or cyclohexane) that is substituted with one or more R¹⁰, wherein at least one R¹⁰ is a C₁₋₆alkyl substituted with —OH. In some embodiments, R³ is a 3-6 membered carbocycle (e.g., a cyclopropane, cyclobutane, cyclopentane, or cyclohexane) that is substituted with one or more R¹⁰, wherein at least one R¹⁰ is —C(O)N(R¹²)₂. In some embodiments, R³ is a cyclopentane that is substituted with one or more R¹⁰, wherein at least one R¹⁰ is —C(O)N(R¹²)₂. In some embodiments, R³ is a 3-6 membered carbocycle (e.g., a cyclopropane, cyclobutane, cyclopentane, or cyclohexane) that is substituted with one or more R¹⁰, wherein at least one R¹⁰ is an unsubstituted C₁₋₆alkyl.

In some embodiments for a compound according to Formula I or I-a, R³ is a spirocycle that is unsubstituted or is substituted with one or more R¹⁰. In some embodiments, R³ is a spirocycle that is unsubstituted or is substituted with one or more R¹⁰, wherein the spirocycle comprises a 4-membered ring (e.g., a cyclobutane). In some embodiments, R³ is a spirocycle that is unsubstituted or is substituted with one or more R¹⁰, wherein the spirocycle comprises a 5-membered ring (e.g., a cyclopentane). In some embodiments, R³ is a spirocycle that is unsubstituted or is substituted with one or more R¹⁰, wherein the spirocycle comprises a 6-membered ring (e.g., a cyclohexane). In some embodiments, R³ is a spirocycle that is unsubstituted or is substituted with one or more R¹⁰, wherein the spirocycle comprises a 4-membered ring (e.g., a cyclobutane) and a 5-membered ring (e.g., a cyclopentane). In some embodiments, R³ is a spirocycle that is unsubstituted or is substituted with one or more R¹⁰, wherein the spirocycle comprises a 5-membered ring (e.g., a cyclopentane) and a 5-membered ring (e.g., a cyclopentane). In some embodiments, R³ is a spirocycle that is unsubstituted or is substituted with one or more R¹⁰, wherein the spirocycle comprises a 6-membered ring (e.g., a cyclohexane) and a 5-membered ring (e.g., a cyclopentane). In some embodiments, R³ is a spirocycle that is unsubstituted or is substituted with one or more R¹⁰, wherein the spirocycle comprises a 6-membered ring (e.g., a cyclohexane) and a 6-membered ring (e.g., a cyclohexane). In some embodiments, R³ is a spirocycle that is unsubstituted or is substituted with one or more R¹⁰, wherein at least one of the one or more R¹⁰ is —OR². In some embodiments, R is a spirocycle that is unsubstituted or is substituted with one or more R¹⁰, wherein the spirocycle comprises a 4-membered ring (e.g., a cyclobutane) and a 5-membered ring (e.g., a cyclopentane), and at least one of the one or more R¹⁰ is —OR¹².

In some embodiments for a compound according to Formula I or 1-a, R³ is a bridged carbocycle that is unsubstituted or is substituted with one or more R¹⁰. In some embodiments, R³ is a bridged carbocycle that is unsubstituted or is substituted with one or more R¹⁰, wherein the bridged carbocycle comprises a 4-membered ring (e.g., a cyclobutane). In some embodiments, R³ is a bridged carbocycle that is unsubstituted or is substituted with one or more R¹⁰, wherein the bridged carbocycle comprises a 5-membered ring (e.g., a cyclopentane). In some embodiments, R³ is a bridged carbocycle that is unsubstituted or is substituted with one or more R¹⁰, wherein the bridged carbocycle comprises a 6-membered ring (e.g., a cyclohexane). In some embodiments, R³ is a bridged carbocycle that is unsubstituted or is substituted with one or more R¹⁰, wherein the one or more R¹⁰ is C₁₋₆alkyl that is unsubstituted or substituted with one or more R²⁰.

In some embodiments for a compound according to Formula I or I-a, when R¹⁰ is —C(O)N(R¹⁴)₂, then at least one R¹⁴ is not H. In some embodiments, when R¹⁰ is —C(O)N(R¹⁴)₂, then each R¹⁴ is C₁₋₆ alkyl (e.g., methyl or ethyl).

In some embodiments for a compound according to Formula I or 1-a, R³ is selected from:

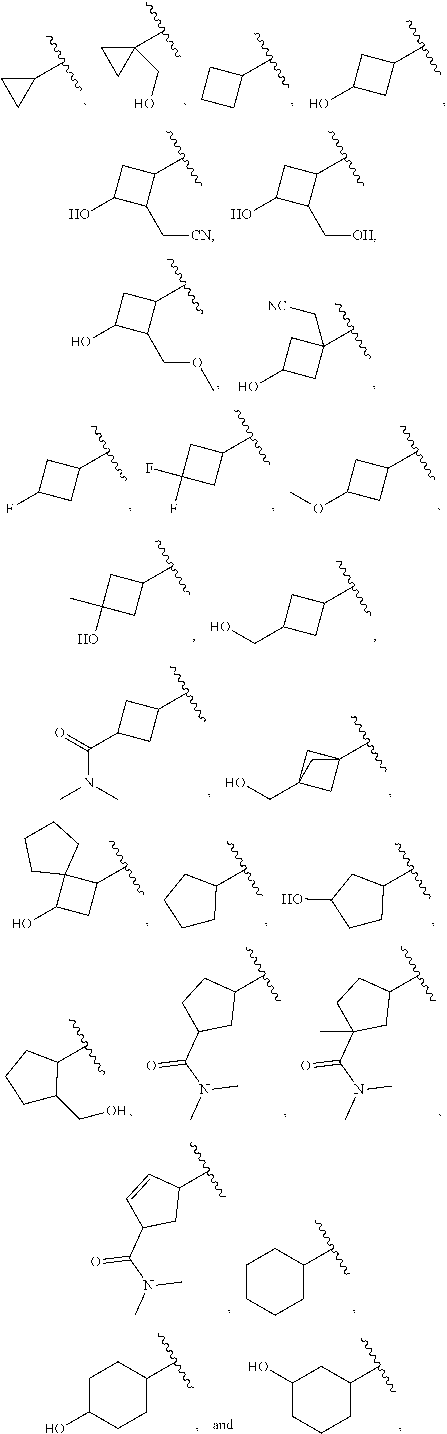

any of which is optionally further substituted with one or more R¹⁰.

In some embodiments, the compound is a compound according to Formula IA, IB, IC, ID, IE, or IF:

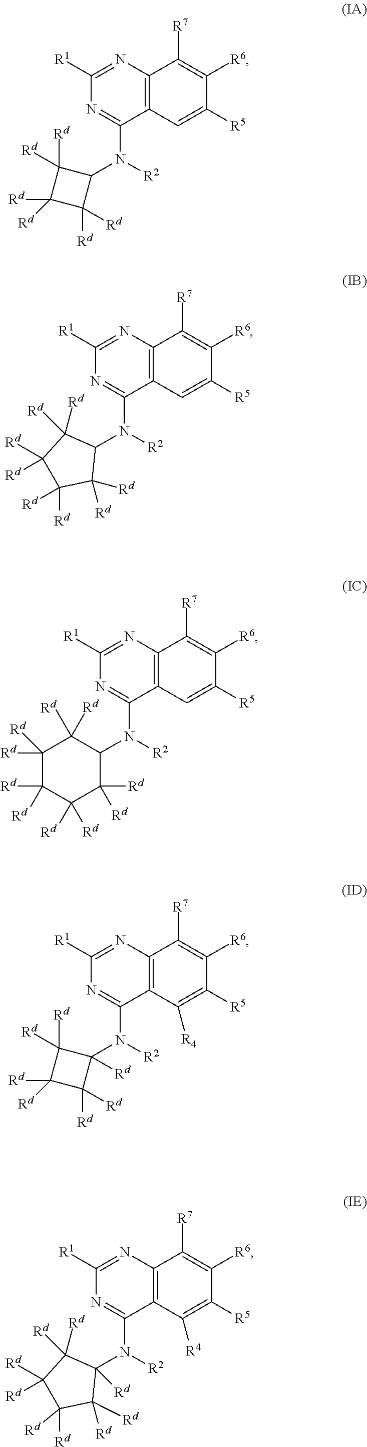

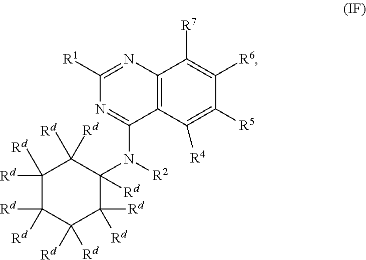

or a salt (e.g., pharmaceutically acceptable salt), ester, tautomer, prodrug, zwitterionic form, or stereoisomer(s) thereof, wherein:
   R¹ is selected from —OR⁸ and

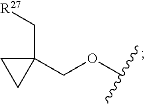

R² is selected from H and C₁₋₆alkyl, wherein any C₁₋₆ alkyl is unsubstituted or is substituted with one or more R¹³;
R⁴, when present, is H;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁶ is a bicyclic heteroaryl substituted with one or more R¹⁵;
R⁷ is selected from halogen, —CN, and H;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹³;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²;
each R²⁸ is independently selected from C₁₋₆alkyl and halogen;
R^{a} and R^{b} are each independently selected from deuterium, halogen, C₁₋₆ alkyl, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³; and
each R^{d} is independently selected from H, —OR¹², —C(O)(C₁₋₆alkylene)CN, —C(O)(C₁₋₆alkylene)OH, —C(O)(C₁₋₆alkyl), —C(O)N(R¹⁴)₂, —S(O)₂(C₁₋₆ alkyl), halogen, and C₁₋₆alkyl, wherein any C₁₋₆alkyl is unsubstituted or substituted with one or more R²⁰.

In some embodiments, the compound is a compound according to Formula IA, or a salt (e.g., pharmaceutically acceptable salt), ester, tautomer, prodrug, zwitterionic form, or stereoisomer(s) thereof.

In some embodiments, the compound is a compound according to Formula IB, or a salt (e.g., pharmaceutically acceptable salt), ester, tautomer, prodrug, zwitterionic form, or stereoisomer(s) thereof.

In some embodiments, the compound is a compound according to Formula IC, or a salt (e.g., pharmaceutically acceptable salt), ester, tautomer, prodrug, zwitterionic form, or stereoisomer(s) thereof.

In some embodiments, the compound is a compound according to Formula ID, or a salt (e.g., pharmaceutically acceptable salt), ester, tautomer, prodrug, zwitterionic form, or stereoisomer(s) thereof.

In some embodiments, the compound is a compound according to Formula IE, or a salt (e.g., pharmaceutically acceptable salt), ester, tautomer, prodrug, zwitterionic form, or stereoisomer(s) thereof.

In some embodiments, the compound is a compound according to Formula IF, or a salt (e.g., pharmaceutically acceptable salt), ester, tautomer, prodrug, zwitterionic form, or stereoisomer(s) thereof.

In some embodiments, the compound is a compound according to Formula IA, or a salt (e.g., pharmaceutically acceptable salt) thereof. In some embodiments, the compound is a compound according to Formula IB, or a salt (e.g., pharmaceutically acceptable salt) thereof. In some embodiments, the compound is a compound according to Formula IC, or a salt (e.g., pharmaceutically acceptable salt) thereof.

In some embodiments, the compound is a compound according to Formula ID, or a salt (e.g., pharmaceutically acceptable salt) thereof. In some embodiments, the compound is a compound according to Formula IE, or a salt (e.g., pharmaceutically acceptable salt) thereof. In some embodiments, the compound is a compound according to Formula IF, or a salt (e.g., pharmaceutically acceptable salt) thereof.

In some embodiments, for a compound according to any one of Formulas I, IA, IB, IC, ID, IE, and IF, R⁶ is selected from:

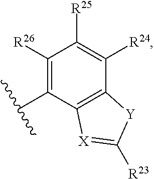

wherein X is selected from N and C—CN; Y is selected from O and S; R²³ is selected from —N(R¹²)₂, C₁₋₆alkyl, and C₁₋₆alkyl-N(R¹²)₂, wherein any C₁₋₆alkyl is unsubstituted or substituted with one or more R¹³; and R²⁴, R²⁵, and R²⁶ are independently selected from H, deuterium, halogen, and C₁₋₆akyl, wherein any C₁₋₆alkyl is unsubstituted or substituted with one or more R¹³. In some embodiments, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R¹²)₂. In some embodiments, X is C—CN, Y is S, and R²³ is —NH₂. In some embodiments, R²⁰ is halogen (e.g., fluoro). In some embodiments, R²⁶ is deuterium.

In some embodiments, for a compound according to any one of Formulas I, IA, IB, IC, ID, IE, and IF, R⁶ is selected from:

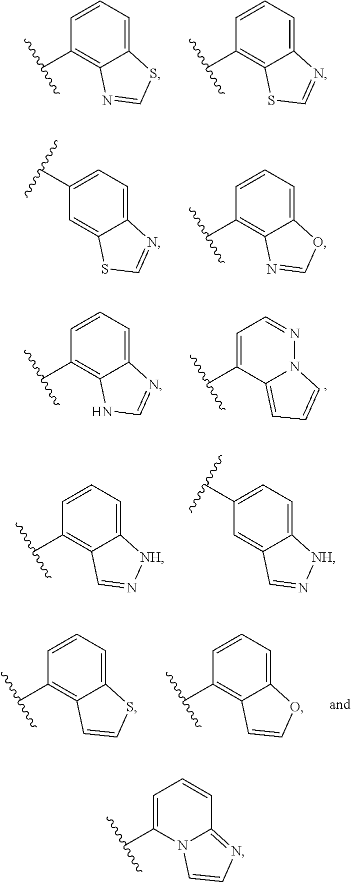

any of which is substituted with one or more R¹⁵.

In some embodiments, for a compound according to any one of Formulas I, IA, IB, IC, ID, IE, and IF, R⁶ is selected from:

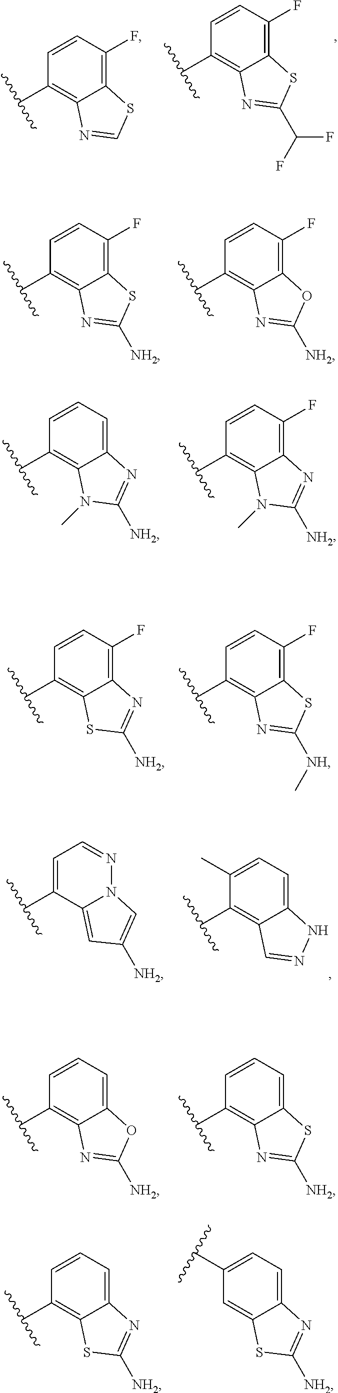

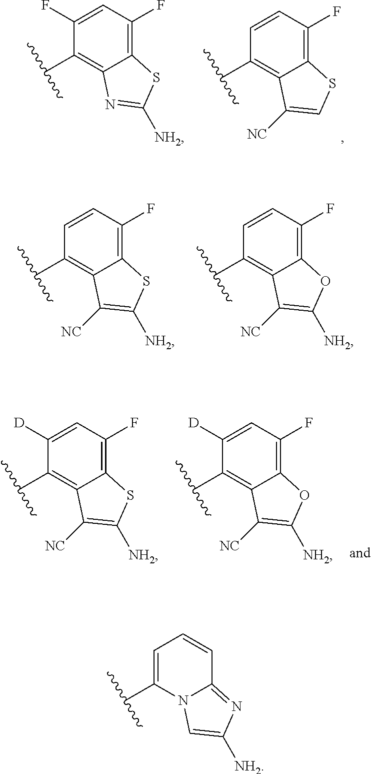

In some embodiments, for a compound according to any one of Formulas I, IA, IB, IC, ID, IE, and IF, R⁶ is selected from:

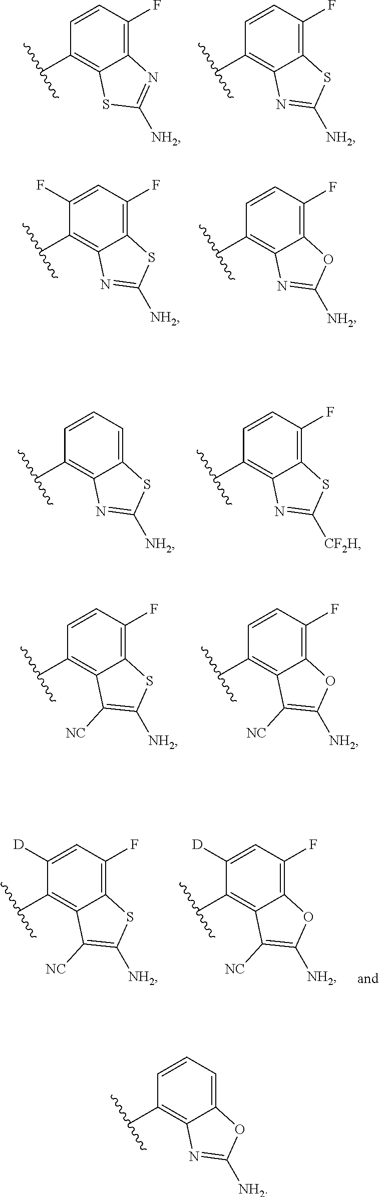

In some embodiments, for a compound according to any one of Formulas I, IA, IB, IC, ID, IE, and IF, R⁶ is

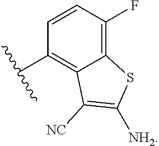 [SMILES: *c1ccc(F)c2sc(N)c(C#N)c12]

In some embodiments, the compound is a compound according to Formula IA1, IB1, IC1, ID1, IE1, and IF1:

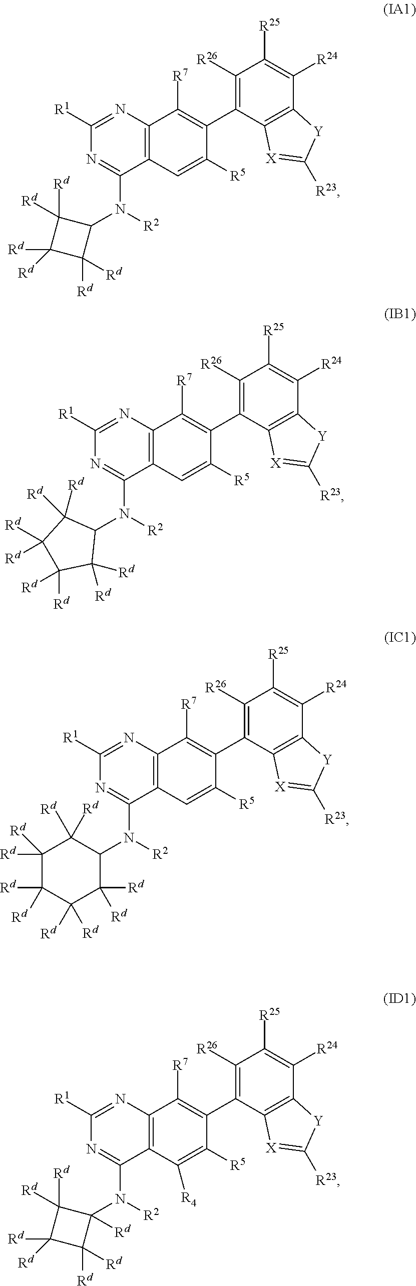

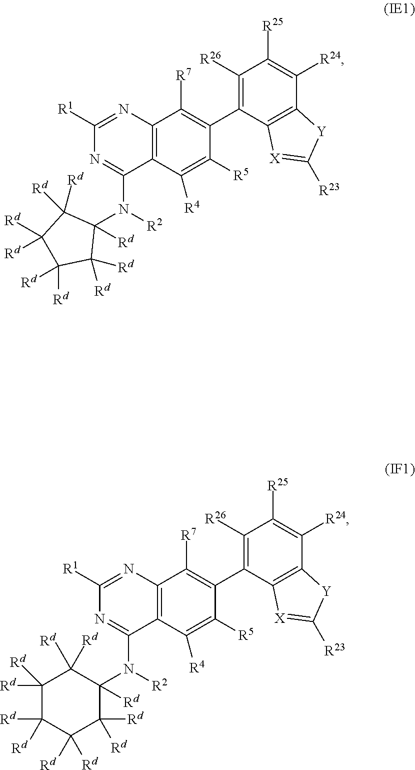

or a salt (e.g., pharmaceutically acceptable salt), ester, tautomer, prodrug, zwitterionic form, or stereoisomer(s) thereof, wherein:
   R¹ is selected from —OR⁸ and

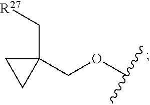

R² is selected from H and C₁₋₆alkyl, wherein any C₁₋₆ alkyl is unsubstituted or is substituted with one or more R¹³;
R⁴, when present, is H;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁷ is selected from halogen, —CN, and H;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹² is independently selected from C₁₋₆alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆alkyl, C₂₋₆ alkenyl, and H;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen;
R^{a} and R^{b} are each independently selected from deuterium, halogen, C₁₋₆ alkyl, —OR¹², and H, wherein an R^{a} and R^{b} optionally join together to form a 3-6 membered carbocycle or heterocycle, and wherein any C₁₋₆alkyl is unsubstituted or is substituted with one or more R¹³;
each R^{d} is independently selected from H, —OR¹², —C(O)(C₁₋₆alkylene)CN, —C(O)(C₁₋₆ alkylene)OH, —C(O)(C₁₋₆alkyl), —C(O)N(R¹²); —S(O)(C₁₋₆alkyl), halogen, and C₁₋₆alkyl, wherein any C₁₋₆alkyl is unsubstituted or substituted with one or more R²⁰;
X is selected from N and C—CN;
Y is selected from O and S;
R² is selected from —N(R¹²)₂, C₁₋₆alkyl, and C₁₋₆alkyl-N(R¹²)₂, wherein any C₁₋₆alkyl is unsubstituted or substituted with one or more R¹³; and
R²⁴, R²⁵, and R²⁶ are independently selected from H, deuterium, halogen, and C₁₋₆alkyl, wherein any C₁₋₆alkyl is unsubstituted or substituted with one or more R¹³.

In some embodiments, the compound is a compound according to Formula IA1, or a salt (e.g., pharmaceutically acceptable salt), ester, tautomer, prodrug, zwitterionic form, or stereoisomer(s) thereof. In some embodiments, the compound is a compound according to Formula IB1, or a salt (e.g., pharmaceutically acceptable salt), ester, tautomer, prodrug, zwitterionic form, or stereoisomer(s) thereof. In some embodiments, the compound is a compound according to Formula IC1, or a salt (e.g., pharmaceutically acceptable salt), ester, tautomer, prodrug, zwitterionic form, or stereoisomer(s) thereof. In some embodiments, the compound is a compound according to Formula ID1, or a salt (e.g., pharmaceutically acceptable salt), ester, tautomer, prodrug, zwitterionic form, or stereoisomer(s) thereof. In some embodiments, the compound is a compound according to Formula IE1, or a salt (e.g., pharmaceutically acceptable salt), ester, tautomer, prodrug, zwitterionic form, or stereoisomer(s) thereof. In some embodiments, the compound is a compound according to Formula IF1, or a salt (e.g., pharmaceutically acceptable salt), ester, tautomer, prodrug, zwitterionic form, or stereoisomer(s) thereof. In some embodiments, the compound is a compound according to Formula IA1, or a salt (e.g., pharmaceutically acceptable salt) thereof. In some embodiments, the compound is a compound according to Formula IB1, or a salt (e.g., pharmaceutically acceptable salt) thereof. In some embodiments, the compound is a compound according to Formula IC1, or a salt (e.g., pharmaceutically acceptable salt) thereof. In some embodiments, the compound is a compound according to Formula ID1, or a salt (e.g., pharmaceutically acceptable salt) thereof. In some embodiments, the compound is a compound according to Formula IE1, or a salt (e.g., pharmaceutically acceptable salt) thereof. In some embodiments, the compound is a compound according to Formula IF1, or a salt (e.g., pharmaceutically acceptable salt) thereof.

In some embodiments, for a compound according to any one of Formulas IA1, IB1, IC1, ID1. IE1, and IF1, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R¹²)₂. In some embodiments, X is C—CN, Y is S, and R²³ is —NH₂. In some embodiments, X is C—CN, Y is S, R²³ is —N(R¹²)₂, and R²⁰ is a halogen (e.g., F). In some embodiments, X is C—CN, Y is S, R²³ is —N(R¹²)₂, and one or more of R″, R″, and R is a halogen (e.g., F). In some embodiments, R¹ is deuterium.

In some embodiments, for a compound according to any one of Formulas I, IA, IA1, IB, IB1, IC, IC1, ID, ID1, IE, IE1, IF, and IF1, R¹ is selected from —OR¹⁴, wherein R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl. In some embodiments, R⁸ is a heterocycle or an alkylheterocycle, wherein any heterocycle contains 4-8 members and is substituted with one or more R^{a} or R^{b}. In some embodiments, R⁸ is a heterocycle that is unsubstituted or substituted with one or more R^{a} or R^{b}. In some embodiments, R⁸ is an alkylheterocycle that is unsubstituted or substituted with one or more R^{a} or R^{b}. In some embodiments, R⁸ is —CH₂(heterocycle), where the heterocycle is unsubstituted or substituted with one or more R^{a} or R^{b}. In some embodiments, a heterocycle or a heterocycle of an alkylheterocycle is a 4-6 membered monocyclic heterocycle having 1-2 heteroatoms independently selected from N, O, and S. In some embodiments, a heterocycle or a heterocycle of an alkylheterocycle is an 8-membered bicyclic heterocycle having 1-2 heteroatoms independently selected from N, O, and S. In some embodiments, a heterocycle or a heterocycle of an alkylheterocycle is substituted with one or more R^{a} or R^{b}, wherein the one or more R^{a} or R^{b} is a halogen (e.g., F). In some embodiments, a heterocycle or a heterocycle of an alkylheterocycle is substituted with one or more R^{a} or R^{b}, wherein the one or more R^{a} or R^{b} is a C₁₋₆alkyl (e.g., methyl). In some embodiments, a heterocycle or a heterocycle of an alkylheterocycle is substituted with one or more R^{a} or R^{b}, wherein the one or more R^{a} or R^{b} is a —OR² (e.g., —OCH₃). In some embodiments, a heterocycle or a heterocycle of an alkylheterocycle is substituted with one or more R^{a} or R^{b}, wherein the one or more R^{a} or R^{b} is a 3-6 membered carbocycle (e.g., a cyclopropane). In some embodiments, a heterocycle or a heterocycle of an alkylheterocycle is substituted with one or more R^{a} or R^{b}, wherein the one or more R^{a} or R^{b} is deuterium.

In some embodiments, for a compound according to any one of Formulas I, IA, IA1, IB, IB1, IC, IC1, ID, ID1, IE, IE1, IF, and IF1, R¹ is selected from:

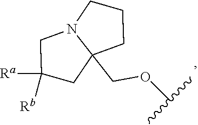

wherein Rand Rare each independently selected from halogen, C₁₋₆alkyl, —OR¹², and H, wherein any C₁₋₆ alkyl is unsubstituted or is substituted with one or more R¹³. In some embodiments, R^{a} is a halogen. In some embodiments, R^{a} is F. In some embodiments, R^{a} is C₁₋₆alkyl that is unsubstituted or is substituted with one or more R¹³. In some embodiments, R^{a} is methyl. In some embodiments, R^{a} is —OC₁₋₆alkyl. In some embodiments, R^{a} is H. In some embodiments, R^{a} is H. In some embodiments, R^{a} is a halogen. In some embodiments, R^{a} is F. In some embodiments, R^{b} is C₁₋₆alkyl that is unsubstituted or is substituted with one or more R¹³. In some embodiments, R^{b} is methyl. In some embodiments, each of R^{a} and R^{b} is F. In some embodiments, each of R^{a} and R^{b} is methyl. In some embodiments, R¹ is selected from:

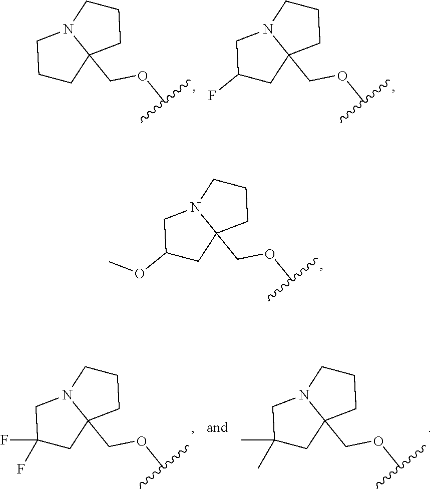 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

In some embodiments, for a compound according to any one of Formulas I, IA, IA1, IB, IB1, IC, IC1, ID, ID1, IE, IE1, IF, and IF1, R¹ is selected from:

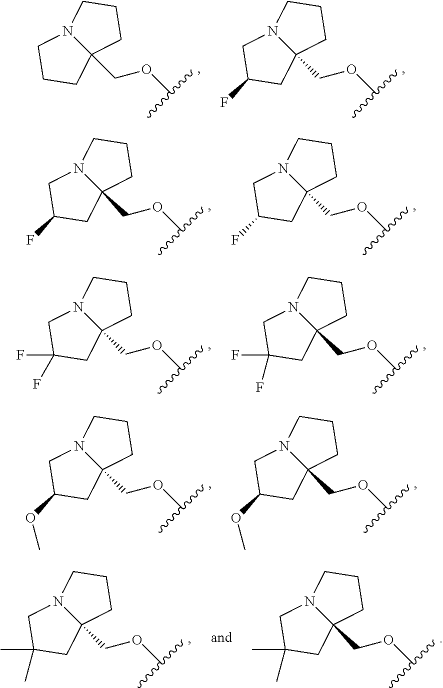

In some embodiments, for a compound according to any one of Formulas I, IA, IA1, IB, IB1, IC, IC1, ID, ID1, IE, IE1, IF, and IF1, R¹ is selected from:

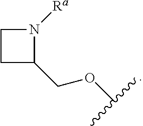

In some embodiments, R^{a} is C₁₋₆ alkyl that is unsubstituted or is substituted with one or more R¹³. In some embodiments, R⁵ is methyl. In some embodiments, R¹ is selected from:

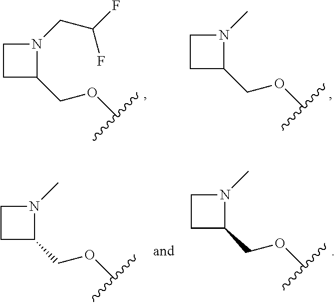

In some embodiments, for a compound according to any one of Formulas I, IA, IA1, IB, IB1, IC, IC1, ID, ID1, IE, IE1, IF, and IF1, R¹ is selected from:

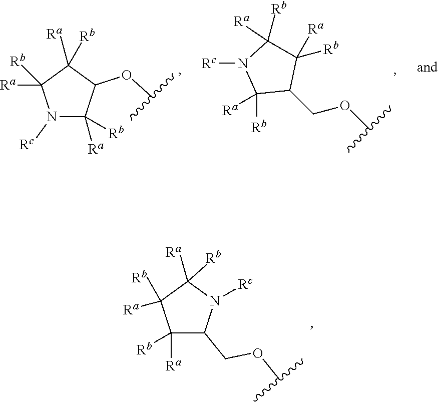

wherein each R^{a} and R^{b} is independently selected from halogen, C₁₋₆alkyl, —OR¹², and H; and R^{e} is selected from C₁₋₆ alkyl, wherein any C₁₋₆ alkyl is unsubstituted or is substituted with one or more R¹³, and wherein an R^{a} and R^{b} or R^{c} optionally join together to form a 3-6 membered carbocycle or heterocycle. In some embodiments, each R^{a} and R^{b} is independently selected from halogen, C₁₋₆alkyl, —OR¹⁴, and H and R^{c} is selected from C₁₋₆ alkyl, wherein any C₁₋₆alkyl is unsubstituted or is substituted with one or more R¹³, and wherein an R^{a} and R^{b} attached to the same carbon atom join together to form a 3-6 membered carbocycle. In some embodiments, each R^{a} and R^{b} is independently selected from halogen, C₁₋₆ alkyl, —OR¹⁴, and H, wherein an R^{a} and R^{b} join together to form a 3-6 membered heterocycle. In some embodiments, each R^{a} and R^{b} is independently selected from halogen, C₁₋₆ alkyl, —OR¹², and H; and R^{c} is selected from C₁₋₆ alkyl, wherein any C₁₋₆ alkyl is unsubstituted or is substituted with one or more R¹³. In some embodiments, one R^{a} or R^{b} is selected from halogen, C₁₋₆ alkyl, and —OR¹², and the other R^{a} and R^{b} groups are H. In some embodiments, one R^{a} or R^{b} is halogen (e.g., F). In some embodiments, two R^{a} groups, two R^{b} groups, or an R^{a} and an R^{b} are halogen (e.g., F). In some embodiments, one R^{a} or R^{b} is —OR¹² (e.g., —OCH₃ or —CHF₂). In some embodiments, one R^{a} or R^{b} is C₁₋₆alkyl (e.g., methyl). In some embodiments, two R^{a} groups, two R^{b} groups, or an R^{a} and an R^{b} are C₁₋₆ alkyl (e.g., methyl). In some embodiments, R^{e} is selected from —CH₃, —CH₂CH₂F, —CH₂CHF₂, and —CH₂CH₂CN. In some embodiments, an R^{a} and R^{b} join together to form a 3-6 membered carbocycle, such as a cyclopropane. In some embodiments, an R^{a} and R^{b} attached to the same carbon atom join together to form a 3-6 membered carbocycle, such as a cyclopropane. In some embodiments, an R^{a} and R^{b} join together to form a 3-6 membered heterocycle. In some embodiments, R¹ is selected from:

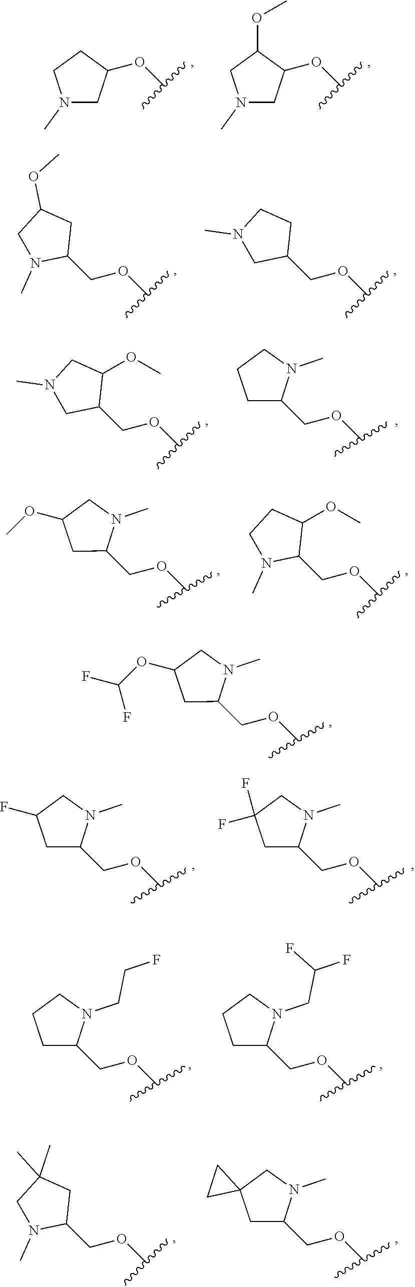

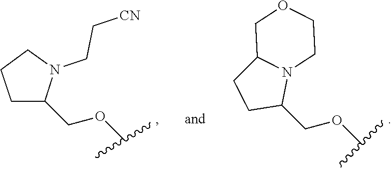 [SMILES: *OCC1CCCN1CCC#N ; *OCC1CCC2COCCN12]

In some embodiments, for a compound according to any one of Formulas I, IA, IA1, IB, IB1, IC, IC1, ID, ID1, IE, IE1, IF, and IF1, R¹ is selected from:

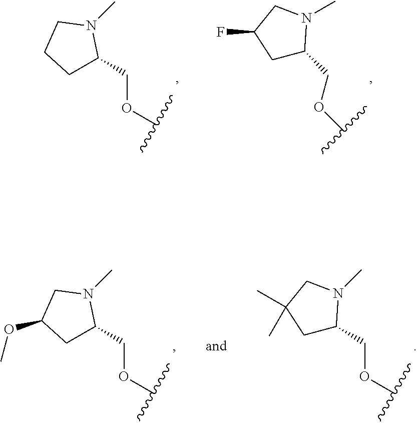

In some embodiments, for a compound according to any one of Formulas I, IA, IA1, IB, IB1, IC, IC1, ID, ID1, IE, IE1, IF, and IF1, R¹ is selected from:

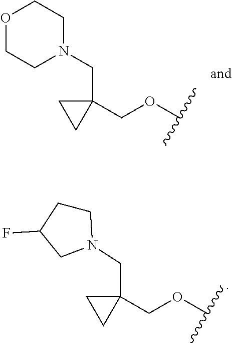 [SMILES: *OCC1(CN2CCOCC2)CC1 ; *OCC1(CN2CCC(F)C2)CC1]

In some embodiments, the compound is a compound according to Formula IA2, IB2, IC2, ID2, IE2, or IF2:

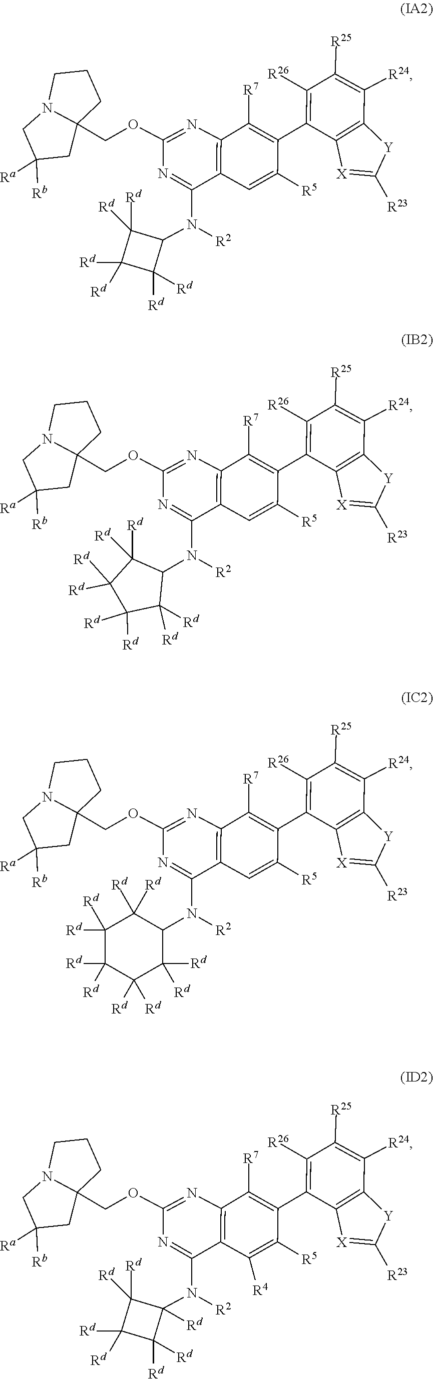

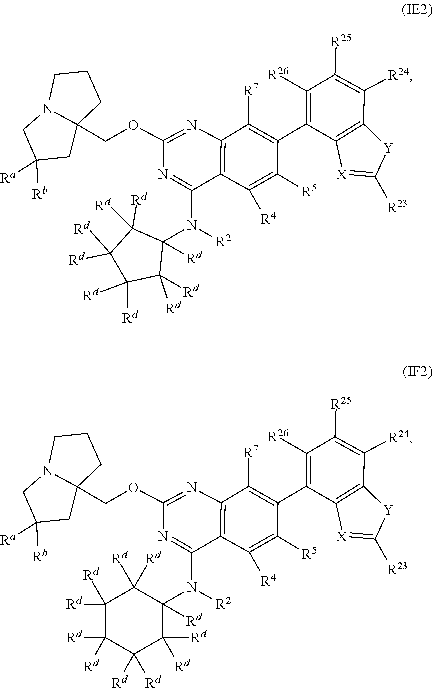

or a salt (e.g., pharmaceutically acceptable salt), ester, tautomer, prodrug, zwitterionic form, or stereoisomer(s) thereof, wherein:
   R² is selected from H and C₁₋₆alkyl, wherein any C₁₋₆ alkyl is unsubstituted or is substituted with one or more R¹³;
   R⁴, when present, is H;
   R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
   R¹ is selected from halogen, —CN, and H;
   each R¹² is independently selected from C₁₋₆ alkyl, C₂₋₆ alkenyl, and H, wherein any C₁₋₆alkyl or C₂₋₆alkenyl is unsubstituted or substituted with one or more R¹³;
   each R³ is independently selected from —OR¹⁴, —CN, —N(R¹⁴)₂, and halogen;
   each R¹⁴ is independently selected from C₁₋₆ alkyl, C₂₋₆ alkenyl, and H;
   each R²⁰ is independently selected from —OH, —OC₁₋₆alkyl, —CN, —NH₂, —NHC₁₋₆alkyl, and halogen;
   R^{a} and R^{b} are each independently selected from halogen, C₁₋₆ alkyl, —OR¹², and H, wherein any C₁₋₆alkyl is unsubstituted or is substituted with one or more R¹³;
   each R^{d} is independently selected from H, —OR¹⁴, —C(O)(C₁₋₆alkylene)CN, —C(O)(C₁₋₆ alkylene)OH, —C(O)(C₁₋₆alkyl), —C(O)N(R¹⁴)₂, —S(O)₂(C₁₋₆alkyl), halogen, and C₁₋₆alkyl, wherein any C₁₋₆alkyl is unsubstituted or substituted with one or more R²⁰
   X is selected from N and C—CN;
   Y is selected from O and S;
   R² is selected from —N(R¹²)₂, C₁₋₆alkyl, and C₁₋₆alkyl-N(R¹⁴)₂, wherein any C₁₋₆alkyl is unsubstituted or substituted with one or more R¹³; and
   R²⁴, R²⁵, and R²⁶ are independently selected from H, deuterium, halogen, and C₁₋₆alkyl, wherein any C₁₋₆alkyl is unsubstituted or substituted with one or more R¹³.

In some embodiments, the compound is a compound according to Formula IA2, or a salt (e.g., pharmaceutically acceptable salt), ester, tautomer, prodrug, zwitterionic form, or stereoisomer(s) thereof. In some embodiments, the compound is a compound according to Formula IB2, or a salt (e.g., pharmaceutically acceptable salt), ester, tautomer, prodrug, zwitterionic form, or stereoisomer(s) thereof. In some embodiments, the compound is a compound according to Formula IC2, or a salt (e.g., pharmaceutically acceptable salt), ester, tautomer, prodrug, zwitterionic form, or stereoisomer(s) thereof. In some embodiments, the compound is a compound according to Formula ID2, or a salt (e.g., pharmaceutically acceptable salt), ester, tautomer, prodrug, zwitterionic form, or stereoisomer(s) thereof. In some embodiments, the compound is a compound according to Formula IE2, or a salt (e.g., pharmaceutically acceptable salt), ester, tautomer, prodrug, zwitterionic form, or stereoisomer(s) thereof. In some embodiments, the compound is a compound according to Formula IF2, or a salt (e.g., pharmaceutically acceptable salt), ester, tautomer, prodrug, zwitterionic form, or stereoisomer(s) thereof. In some embodiments, the compound is a compound according to Formula IA2, or a salt (e.g., pharmaceutically acceptable salt) thereof. In some embodiments, the compound is a compound according to Formula IB2, or a salt (e.g., pharmaceutically acceptable salt) thereof. In some embodiments, the compound is a compound according to Formula IC2, or a salt (e.g., pharmaceutically acceptable salt) thereof. In some embodiments, the compound is a compound according to Formula ID2, or a salt (e.g., pharmaceutically acceptable salt) thereof. In some embodiments, the compound is a compound according to Formula IE2, or a salt (e.g., pharmaceutically acceptable salt) thereof. In some embodiments, the compound is a compound according to Formula IF2, or a salt (e.g., pharmaceutically acceptable salt) thereof.

In some embodiments, for a compound according to any one of Formulas IA2, IB2, IC2, ID2, IE2, and IF2, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R¹²)₂. In some embodiments, X is C—CN, Y is S, and R²³ is —NH₂. In some embodiments, X is C—CN, Y is S, R²³ is —N(R¹²)₂, and R²⁰ is a halogen (e.g., F). In some embodiments, X is C—CN, Y is S, R²³ is —N(R¹²)₂, and one or more of R²⁴, R²⁵, and R²⁶ is a halogen (e.g., F). In some embodiments, R²⁶ is deuterium.

In some embodiments, for a compound according to any one of Formulas IA2, IB2, IC2, ID2, IE2, and IF2, R^{a} is a halogen. In some embodiments, R^{a} is F. In some embodiments, R^{a} is C₁₋₆alkyl that is unsubstituted or is substituted with one or more R¹³. In some embodiments, R^{a} is methyl. In some embodiments, R^{a} is —OC₁₋₆alkyl. In some embodiments, R^{a} is H. In some embodiments, R^{b} is H. In some embodiments, R^{b} is a halogen. In some embodiments, R^{b} is F. In some embodiments, R^{a} is C₁₋₆alkyl that is unsubstituted or is substituted with one or more R¹³. In some embodiments, R^{b} is methyl. In some embodiments, each of R^{a} and R^{b} is F. In some embodiments, each of R^{a} and R^{b} is methyl.

In some embodiments, for a compound according to any one of Formulas IA, IA1, IA2, IB, IB1, IB2, IC, IC1, IC2, ID, ID1, ID2, IE, IE1, IE2, IF, IF1, and IF2, at least one R^{d} is selected from —OR¹², —C(O)(C₁₋₆alkylene)CN, —C(O)(C₁₋₆alkylene)OH, —C(O)(C₁₋₆alkyl), —C(O)N(R¹²)₂, —S(O)₂(C₁₋₆alkyl), halogen, and C₁₋₆alkyl, wherein any C₁₋₆alkyl is unsubstituted or substituted with one or more R². In some embodiments, at least one R^{d} is selected from —OR¹², —C(O)(C₁₋₆alkylene)CN, —C(O)(C₁₋₆alkylene)OH, —C(O)(C₁₋₆alkyl), —C(O)N(R¹²)₂, —S(O)₂(C₁₋₆alkyl), halogen, and C₁₋₆alkyl that is unsubstituted or substituted with one or more R²⁰. In some embodiments, at least one R^{d} is selected from —OR¹², —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl that is unsubstituted or substituted with one or more R²⁰. In some embodiments, at least one R^{d} is selected from —OR² and C₁₋₆alkyl, wherein any C₁₋₆alkyl is unsubstituted or substituted with one or more R²⁰. In some embodiments, at least one R^{d} is —OH. In some embodiments, at least one R^{d} is a halogen (e.g., F). In some embodiments, at least one R^{d} is —C(O)N(R¹⁴)₂. In some embodiments, at least one R^{d} is —C(O)N(R¹⁴)₂, wherein each R″ is independently selected from C₁₋₆alkyl. In some embodiments, at least one R^{d} is —C(O)N(CH₃)₂. In some embodiments, when R^{d} is —C(O)N(R¹⁴)₂, then at least one R¹⁴ is not H. In some embodiments, when R^{d} is —C(O)N(R¹⁴)₂, then each R″ is C₁₋₆ alkyl (e.g., methyl or ethyl). In some embodiments, each R^{d} is H.

In some embodiments, for a compound according to any one of Formulas I, I-a, IA. IA1, IA2, IB, IB1, IB2, IC, IC1, IC2, ID, ID1, ID2, IE, IE1, IE2, IF, IF1, and IF2, R² is H. In some embodiments, R² is C₁₋₆alkyl that is unsubstituted or is substituted with one or more R¹³. In some embodiments, R² is C₁₋₆alkyl that is unsubstituted. In some embodiments, R² is C₁₋₆alkyl that is substituted with one or more R¹³. In some embodiments, R² is C₁₋₂alkyl that is unsubstituted or is substituted with one or more R¹³. In some embodiments, R² is C₁₋₂alkyl that is unsubstituted. In some embodiments, R² is C₁₋₂alkyl that is substituted with one or more R¹³. In some embodiments, R² is methyl. In some embodiments, R² is ethyl.

In some embodiments, for a compound according to any one of Formulas I, IA, IA1, IA2, IB, IB1, IB2, IC, IC1, IC2, ID, ID1, ID2, IE, IE1, IE2, IF, IF1, and IF2, R⁵ is H. In some embodiments, R⁵ is a halogen (e.g., F or Cl). In some embodiments, R¹ is C₁₋₆ In some embodiments, R⁵ is F. In some embodiments, R⁵ is —CN. In some embodiments, R¹ is —OR¹², wherein R² is selected from C₁₋₆ alkyl and H. In some embodiments, R⁵ is —OCH₃. In some embodiments, R⁵ is —OCF₃. In some embodiments, R¹ is selected from C₁₋₆alkyl that is unsubstituted or substituted with one or more R¹³. In some embodiments, R¹ is selected from C₁₋₂alkyl that is unsubstituted or substituted with one or more R¹³. In some embodiments, R⁵ is selected from C₁₋₆alkyl that is unsubstituted, such as methyl or ethyl. In some embodiments, R⁵ is selected from C₁₋₆alkyl that is substituted with one or more halogens or —CN. In some embodiments, R⁵ is C₁₋₆alkyl that is substituted with one or more halogens, such as one or more fluorines. In some embodiments, R⁵ is —CF₃. In some embodiments, R⁵ is —CHF₂. In some embodiments, R⁵ is selected from —CF₂H, —CF₃, —CH₂CN, and —CH₂CH₃. In some embodiments, R⁵ is selected from —CH₃, —CH₂CH₃, —CF₂H, —CF₃, —CF₂CH₃, and —CH₂CN. In some embodiments, R³ is C₁₋₆alkyl that is substituted with one or more R¹³, wherein each R¹³ is independently selected from —OR¹⁴, —CN, and —N(R¹²)₂. In some embodiments, R is —CH₂CN. In some embodiments, R¹ is a 3-6 membered heterocycle. In some embodiments, R⁵ is a 5-6 membered heteroaryl, such as a furan.

In some embodiments, for a compound according to any one of Formulas I, IA, IA1, IA2, IB, IB1, IB2, IC, IC1, IC2, ID, ID1, ID2, IE, IE1, IE2, IF, IF1, and IF2, R¹ is H. In some embodiments, R¹ is a halogen (e.g., F or Cl). In some embodiments, R¹ is C₁₋₆ In some embodiments, R⁷ is F. In some embodiments, R⁷ is —CN.

In another aspect, the present disclosure provides a compound represented by Formula II′:

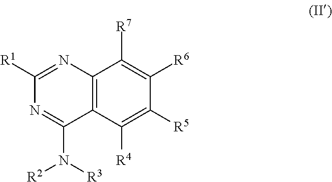

or a salt (e.g., pharmaceutically acceptable salt), ester, tautomer, prodrug, zwitterionic form, or stereoisomer(s) thereof, wherein:

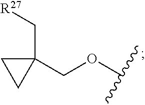

R¹ is selected from —OR⁸ and;
R² is selected from H, C₁₋₆alkyl, and a 3-6 membered carbocycle, wherein any C₁₋₆ alkyl is unsubstituted or is substituted with one or more R¹³;
   R³ is selected from a 4-9 membered heterocycle that is unsubstituted or substituted with one or more R¹⁰;
   R⁴ is H;
   R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
   R⁶ is a monocyclic or bicyclic heteroaryl, wherein the heteroaryl is substituted with one or more R¹⁵;
   R⁷ is selected from halogen;
   R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
   each R¹⁰ is independently selected from deuterium, —OR¹², ═O, —C(O)(C₁₋₆alkylene)CN, —C(O)(C₁₋₆alkylene)OH, —C(O)(C₁₋₆alkyl), —C(O)O(C₁₋₆alkyl), —C(O)(3-6 membered carbocycle), —C(O)(3-8 membered heterocycle), —C(O)(5-6 membered heteroaryl), —C(O)O(3-6 membered carbocycle), —C(O)O(3-6 membered heterocycle), —C(O)O(C₁₋₆alkylene)(3-6 membered heterocycle), —C(O)N(R¹²)₂, —C(O)OR¹⁴, —S(O)₂(C₁₋₆alkyl), halogen, a 5-6 membered heteroaryl, a 3-6 membered carbocycle, a 3-6 membered heterocycle, and C₁₋₆alkyl, wherein any C₁₋₆alkyl is optionally deuterated and is unsubstituted or substituted with one or more R²⁰, wherein any 3-6 membered carbocycle, 5-6 membered heteroaryl, or 3-6 or 3-8 membered heterocycle is unsubstituted or substituted with one or more R¹² or R²⁰, and wherein two R¹⁰s optionally join together to form, together with the atom(s) to which they are attached, a 3-6 membered carbocycle or heterocycle;
   each R¹² is independently selected from C₁₋₆ alkyl, C₂₋₆ alkenyl, and H, wherein any C₁₋₆alkyl or C₂₋₄ alkenyl is unsubstituted or substituted with one or more R¹³;
   each R¹³ is independently selected from —OR¹⁴, —CN, —N(R¹²)₂, and halogen;
   each R¹⁴ is independently selected from a 3-6 membered carbocycle, a 3-6 membered heterocycle, C₁₋₆ alkyl, C₂₋₆ alkenyl, and H, wherein any C₁₋₆ alkyl is optionally deuterated;
   each R⁵ is independently selected from deuterium, halogen, —N(R¹²)₂, —CN, and C₁₋₆alkyl, wherein any C₁₋₆alkyl is unsubstituted or substituted with one or more R¹³;
   each R^{a} is independently selected from —OH, —OC₁₋₆alkyl, —OC₁₋₆haloalkyl, ═O, —CN, —NH₂, —NHC₁₋₆alkyl, —C(O)(C₁₋₆alkyl), a 3-6 membered carbocycle, phenyl, and halogen;
   R²⁷ is a 3-6 membered heterocycle including one or more heteroatoms selected from N, O, and S, wherein the heterocycle is unsubstituted or substituted with one or more R¹³;
   each R²⁸ is independently selected from C₁₋₆alkyl and halogen; and
   R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.

In some embodiments, the present disclosure provides a compound of Formula II′, or a salt (e.g., a pharmaceutically acceptable salt) thereof.

In another aspect, the present disclosure provides a compound represented by Formula II:

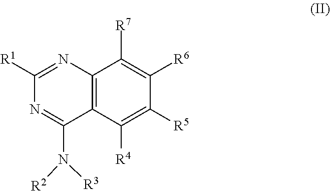

or a salt (e.g., pharmaceutically acceptable salt), ester, tautomer, prodrug, zwitterionic form, or stereoisomer(s) thereof, wherein:
R¹ is selected from —OR⁸ and

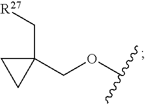

R₂ is selected from H, C₁₋₆alkyl, and a 3-6 membered carbocycle, wherein any C₁₋₆ alkyl is unsubstituted or is substituted with one or more R¹³;
R¹ is selected from a 4-9 membered heterocycle that is unsubstituted or substituted with one or more R¹⁰, provided that (i) when the heterocycle contains a nitrogen atom, the nitrogen atom is substituted with R¹⁰, or (ii) the heterocycle does not comprise an —NH— moiety;
R⁴ is H;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹⁰ is independently selected from deuterium, —OR¹², ═O, —C(O)(C₁₋₆alkylene)CN, —C(O)(C₁₋₆alkylene)OH, —C(O)(C₁₋₆alkyl), —C(O)O(C₁₋₆_alkyl), —C(O)(3-6 membered carbocycle), —C(O)(3-8 membered heterocycle), —C(O)(5-6 membered heteroaryl), —C(O)O(3-6 membered carbocycle), —C(O)O(3-6 membered heterocycle), —C(O)O(C₁₋₆alkylene)(3-6 membered heterocycle), —C(O)N(R¹²)₂, —C(O)OR¹⁴, —S(O)₂(C₁₋₆alkyl), halogen, a 5-6 membered heteroaryl, a 3-6 membered carbocycle, and C₁₋₆alkyl, wherein any C₁₋₆alkyl is optionally deuterated and is unsubstituted or substituted with one or more R²⁰; wherein any 3-6 membered carbocycle, 5-6 membered heteroaryl, or 3-6 or 3-8 membered heterocycle is unsubstituted or substituted with one or more R¹² or R²⁰; and wherein two R¹⁰s optionally join together to form, together with the atom(s) to which they are attached, a 3-6 membered carbocycle or heterocycle;
   each R¹² is independently selected from C₁₋₆alkyl, C₂₋₆ alkenyl, and H, wherein any C₁₋₆alkyl or C₂₋₆ alkenyl is unsubstituted or substituted with one or more R¹³;
   each R¹³ is independently selected from —OR¹⁴, —CN, —N(R¹⁴)₂, and halogen;
   each R¹⁴ is independently selected from a 3-6 membered carbocycle, a 3-6 membered heterocycle, C₁₋₆alkyl, C₂₋₆ alkenyl, and H, wherein any C₁₋₆ alkyl is optionally deuterated;
   each R¹⁵ is independently selected from deuterium, halogen, —N(R¹²)₂, —CN, and C₁₋₆alkyl, wherein any C₁₋₆alkyl is unsubstituted or substituted with one or more R¹³;
   each R²⁰ is independently selected from —OH, —OC₁₋₆alkyl, —OC₁₋₆haloalkyl, —O, —CN, —NH₂, —NHC₁₋₆alkyl, —C(O)(C₁₋₆alkyl), a 3-6 membered carbocycle, phenyl, and halogen;
   R²⁷ is a 3-6 membered heterocycle including one or more heteroatoms selected from N, O, and S, wherein the heterocycle is unsubstituted or substituted with one or more R²;
   each R²⁸ is independently selected from C₁₋₆alkyl and halogen; and
   R^{a} and R^{b} are each independently selected from deuterium, halogen, C₁₋₆ alkyl, —OR¹², a 3-6 membered carbocycle, and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.

In some embodiments, the present disclosure provides a compound of Formula II, or a salt (e.g., a pharmaceutically acceptable salt) thereof.

In some embodiments, the present disclosure provides a compound of Formula II, wherein:
R¹ is —OR¹;
R² is selected from C₁₋₆alkyl and a 3-6 membered carbocycle, wherein any C₁₋₆alkyl is unsubstituted or is substituted with one or more R¹³;
R³ is selected from a 4-9 membered heterocycle that is unsubstituted or substituted with one or more R¹⁰, provided that (i) when the heterocycle contains a nitrogen atom, the nitrogen atom is substituted with R¹⁰, or (ii) the heterocycle does not comprise an —NH— moiety:
R⁴ is H;
R⁵ is selected from halogen and C₁₋₆alkyl, wherein any C₁₋₆alkyl is unsubstituted or substituted with one or more R¹³;
R⁶ is a bicyclic heteroaryl, wherein the heteroaryl is substituted with one or more R¹³;
R⁷ is halogen;
R⁸ is an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹⁰ is independently selected from deuterium, —OR¹², ═O, —C(O)(C₁₋₆alkylene)CN, —C(O)(C₁₋₆alkylene)OH, —C(O)(C₁₋₆alkyl), —C(O)O(C₁₋₆alkyl), —C(O)(3-6 membered carbocycle), —C(O)(3-8 membered heterocycle), —C(O)(5-6 membered heteroaryl), —C(O)O(3-6 membered carbocycle), —C(O)O(3-6 membered heterocycle), —C(O)O(C₁₋₆alkylene)(3-6 membered heterocycle), —C(O)N(R¹²)₂, —S(O)₂(C₁₋₆alkyl), halogen, a 5-6 membered heteroaryl, a 3-6 membered carbocycle, and C₁₋₆alkyl, wherein any C₁₋₆alkyl is optionally deuterated and is unsubstituted or substituted with one or more R²⁰; wherein any 3-6 membered carbocycle, 5-6 membered heteroaryl, or 3-6 or 3-8 membered heterocycle is unsubstituted or substituted with one or more R¹² or R²⁰;
each R² is independently selected from C₁₋₆ alkyl and H, wherein any C₁₋₆alkyl is unsubstituted or substituted with one or more R¹³;
each R¹³ is independently selected from halogen;
each R⁴ is independently selected from a 3-6 membered carbocycle, a 3-6 membered heterocycle, C₁₋₆alkyl, and H;
each R″ is independently selected from halogen, —N(R¹²)₂, and —CN;
each R²⁰ is independently selected from —OH, —OC₆₄alkyl, —OC₁₋₆haloalkyl, —CN, —C(O)(C₁₋₆alkyl), a 3-6 membered carbocycle, phenyl, and halogen; and
R^{a} and R^{b} are each independently selected from deuterium, halogen, C₁₋₆ alkyl, —OR¹², and H, wherein an R^{a} and R^{b} optionally join together to form a 3-6 membered carbocycle.

In some embodiments, the present disclosure provides a compound of Formula II′ or II, wherein the compound is of Formula II-a:

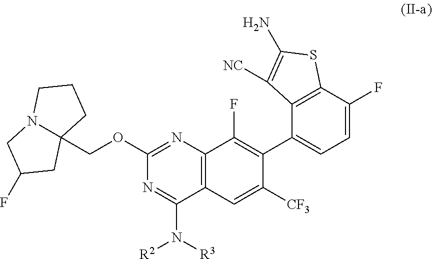

or a salt (e.g., pharmaceutically acceptable salt), ester, tautomer, prodrug, zwitterionic form, or stereoisomer(s) thereof, wherein R² and R³ are as defined for Formula II above and described in classes and subclasses herein, both singly and in combination. In some embodiments, R² and R³ are as defined for Formula II′ above and described in classes and subclasses herein, both singly and in combination. In some embodiments, the present disclosure provides a compound of Formula II-a, or a salt (e.g., pharmaceutically acceptable salt) thereof.

In some embodiments, for a compound according to any one of Formulas II, II′, and II-a, R³ is a 4-6 membered heterocycle that includes one or more heteroatoms selected from O, S, and N, wherein the heterocycle is unsubstituted or is substituted with one or more R¹⁰. In some embodiments, R³ is a 4-6 membered heterocycle that includes one or more heteroatoms selected from O, S, and N, wherein the heterocycle is unsubstituted or is substituted with one or more R¹⁰, provided that when the heterocycle contains a nitrogen atom, the nitrogen atom is substituted with R¹⁰. In some embodiments, R³ is a 4-6 membered heterocycle that includes one heteroatom selected from O and S, wherein the heterocycle is unsubstituted or is substituted with one or more R¹⁰. In some embodiments, R³ is a 4-6 membered heterocycle that includes one heteroatom selected from O, S, and N, wherein the heterocycle is unsubstituted or is substituted with one or more R¹⁰, provided that when the heterocycle contains a nitrogen atom, the nitrogen atom is substituted with R¹⁰. In some embodiments, R³ is a 4-6 membered heterocycle that includes 1 heteroatom selected from O, S, and N, wherein the heterocycle is substituted with 1-4 R¹⁰, provided that when the heterocycle contains a nitrogen atom, the nitrogen atom is substituted with R¹⁰. In some embodiments, R³ is a 4-6 membered heterocycle that includes a nitrogen atom, wherein the heterocycle is substituted with 1-4 R¹⁰, provided that the nitrogen atom is substituted with R¹⁰. In some embodiments, R³ is a 4-6 membered heterocycle that includes a sulfur atom, wherein the heterocycle is substituted with 1-4 R¹⁰. In some embodiments, R¹ is a 4-6 membered heterocycle that includes an oxygen atom, wherein the heterocycle is substituted with 1-4 R¹⁰. In some embodiments, R³ is a pyrrolidine that is substituted with 0-4 R¹⁰, provided that the nitrogen atom of the heterocycle is substituted with R¹⁰. In some embodiments, R³ is a pyrrolidine that is substituted with 1-4 R¹⁰, provided that the nitrogen atom of the heterocycle is substituted with R¹⁰. In some embodiments, R³ is an oxetane or thietane that is substituted with 0-4 R¹⁰. In some embodiments, R³ is an oxetane or thietane that is substituted with 1-4 R¹⁰. In some embodiments, R³ is a tetrahydrofuran or tetrahydrothiophene that is substituted with 0-4 R¹⁰. In some embodiments, R³ is a tetrahydrofuran or tetrahydrothiophene that is substituted with 1-4 R¹⁰. In some embodiments, R³ is a 4-6 membered heterocycle that is substituted with one or more R¹⁰, wherein at least one R¹⁰ is selected from —OR⁸ and a C₁₋₆alkyl substituted with —OH, provided that when the heterocycle contains a nitrogen atom, the nitrogen atom is substituted with R¹⁰. In some embodiments, R³ is a 4-6 membered heterocycle that is substituted with one or more R¹⁰, wherein at least one R¹⁰ is an unsubstituted C₁₋₆alkyl, provided that when the heterocycle contains a nitrogen atom, the nitrogen atom is substituted with R¹⁰. In some embodiments, R³ is selected from a 4-6 membered heterocycle that is substituted with one or more R¹³, provided that when the heterocycle contains a nitrogen atom, the nitrogen atom is substituted with R¹⁰, wherein each R¹⁰ is independently selected from —OR¹², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl that is unsubstituted or substituted with one or more R²⁰. In some embodiments, R³ is selected from a 4-6 membered heterocycle that is substituted with one or more R¹⁰, provided that when the heterocycle contains a nitrogen atom, the nitrogen atom is substituted with R¹⁰, wherein each R¹⁰ is independently selected from —OR¹², ═O, —C(O)(C₁₋₆ alkylene)CN, —C(O)(C₁₋₆alkylene)OH, —C(O)(C₁₋₆alkyl), —C(O)O(C₁₋₆alkyl), —C(O)(3-6 membered carbocycle), —C(O)(3-7 membered heterocycle), —C(O)(5-6 membered heteroaryl), —C(O)O(3-6 membered heterocycle), —C(O)O(C₁₋₆alkylene)(3-6 membered heterocycle), —C(O)OR¹⁴, —S(O)₂(C₁₋₆alkyl), halogen, a 3-6 membered carbocycle, and C₁₋₆alkyl that is unsubstituted or substituted with one or more R¹⁰, wherein any 3-6 membered carbocycle, 5-6 membered heteroaryl, or 3-6 membered heterocycle is unsubstituted or substituted with one or more R¹² or R²⁰. In some embodiments, R³ is selected from a 4-6 membered heterocycle that is substituted with one or more R¹³, provided that when the heterocycle contains a nitrogen atom, the nitrogen atom is substituted with R, and each R¹⁰ is independently selected from —C(O)(C₁₋₆alkyl), —C(O)(3-6 membered carbocycle), —C(O)O(C₁₋₆alkyl), halogen, and C₁₋₆alkyl, wherein any C₁₋₆alkyl is unsubstituted or substituted with one or more R¹⁰, and any 3-6 membered carbocycle is unsubstituted or substituted with one or more R¹² or R²⁰. In some embodiments, R³ is selected from a 4-6 membered heterocycle that is substituted with one or more R¹³, provided that when the heterocycle contains a nitrogen atom, the nitrogen atom is substituted with R¹⁰, wherein at least one R¹⁰ is selected from —C(O)(C₁₋₆alkyl) and —C(O)O(C₁₋₆alkyl). In some embodiments, two R¹⁰s join together to form, together with the atom(s) to which they are attached, a 3-6 membered carbocycle or heterocycle. In some embodiments, two R¹⁰s connected to adjacent atoms join together to form, together with the atoms to which they are attached, a 3-6 membered carbocycle or heterocycle. In some embodiments, two R¹⁰s connected to adjacent atoms join together to form, together with the atoms to which they are attached, a 5-6 membered heterocycle, such as a pyrrolidine or oxazolidine. In some embodiments, the 3-6 membered carbocycle or heterocycle formed by the joining of two R¹⁰s is substituted with one or more R¹⁰. In some embodiments, the 5-6 membered heterocycle formed by the joining of two R¹⁰s is substituted with one or more R¹⁰. In some embodiments, the 5-6 membered heterocycle (e.g., pyrrolidine or oxazolidine) formed by the joining of two R¹⁰s is substituted with ═O. In some embodiments, each R¹⁰ is independently selected from —OR¹², ═O, —C(O)(C₁₋₆ alkylene)CN, —C(O)(C₁₋₆alkylene)OH, —C(O)(C₁₋₆alkyl), —C(O)(3-6 membered carbocycle), —C(O)N(R¹²)₂, —S(O)₂(C₁₋₆alkyl), halogen, and C₁₋₆alkyl, wherein any C₁₋₆alkyl is unsubstituted or substituted with one or more R²⁰, and wherein two R¹⁰s optionally join together to form, together with the atom(s) to which they are attached, a 3-6 membered carbocycle or heterocycle. In some embodiments, each R¹⁰ is independently selected from —OR¹², ═O, —C(O)(C₁₋₆akylene)CN, —C(O)(C₁₋₆alkylene)OH, —C(O)(C₁₋₆alkyl), —C(O)(3-6 membered carbocycle), —C(O)N(R¹⁴)₂, —S(O)₂(C₁₋₆alkyl), halogen, and C₁₋₆alkyl, wherein any C₁₋₆alkyl is unsubstituted or substituted with one or more R²⁰. In some embodiments, each R¹⁰ is independently selected from —OR¹², —C(O)(C₁₋₆alkylene)CN, —C(O)(C₁₋₆alkylene)OH, —C(O)(C₁₋₆alkyl), halogen, and C₁₋₆alkyl, wherein any C₁₋₆alkyl is unsubstituted or substituted with one or more R²⁰. In some embodiments, each R¹⁰ is independently selected from —C(O)(C₁₋₆alkyl), —C(O)(3-6 membered carbocycle), —C(O)O(C₁₋₆alkyl), halogen, and C₁₋₆alkyl, wherein any C₁₋₆alkyl is unsubstituted or substituted with one or more R²⁰, and any 3-6 membered carbocycle is unsubstituted or substituted with one or more R¹² or R²⁰.

In some embodiments, for a compound according to any one of Formulas II, II′, and II-a, R³ is a 7-9 membered heterocycle that includes one or more heteroatoms selected from O, S, and N, wherein the heterocycle is unsubstituted or is substituted with one or more R¹⁰. In some embodiments, R³ is a 7-9 membered heterocycle that includes one or more heteroatoms selected from O, S, and N, wherein the heterocycle is unsubstituted or is substituted with one or more R¹⁰, provided that (i) when the heterocycle contains a nitrogen atom, the nitrogen atom is substituted with R¹⁰ or (ii) the heterocycle docs not comprise an —NH— moiety. In some embodiments, R³ is a 7-9 membered heterocycle that includes one or more heteroatoms selected from O, S, and N, wherein the heterocycle is unsubstituted or is substituted with one or more R¹⁰, provided that (i) when the heterocycle contains a nitrogen atom, the nitrogen atom is substituted with R¹⁰ or (ii) when the heterocycle contains a single ring, the heterocycle does not comprise an —NH-moiety. In some embodiments, R³ is a 7-9 membered bridged heterocycle that includes one or more heteroatoms selected from O, S, and N, wherein the bridged heterocycle is unsubstituted or is substituted with one or more R¹⁰. In some embodiments, R³ is a 7-9 membered bridged heterocycle that includes one or more heteroatoms selected from O, S, and N, wherein the bridged heterocycle is unsubstituted or is substituted with one or more R¹⁰, provided that (i) when the bridged heterocycle contains a nitrogen atom, the nitrogen atom is substituted with R¹⁰ or (ii) the bridged heterocycle does not comprise an —NH— moiety. In some embodiments, R³ is a bridged pyrrolidine that is unsubstituted or is substituted with one or more R¹⁰. In some embodiments, R³ is a bridged pyrrolidine that is unsubstituted or is substituted with one or more R¹⁰, provided that the bridged pyrrolidine does not comprise an NH moiety. In some embodiments, R³ is a bridged pyrrolidine that is substituted with one or more R¹⁰, provided that the bridged pyrrolidine does not comprise an NH moiety. In some embodiments, R³ is a 7-9 membered heterocycle comprising a fused ring system that includes one or more heteroatoms selected from O, S, and N, wherein the heterocycle is unsubstituted or is substituted with one or more R¹⁰. In some embodiments, R³ is a 7-9 membered heterocycle comprising a fused ring system that includes one or more heteroatoms selected from O, S, and N, wherein the heterocycle is unsubstituted or is substituted with one or more R¹³, provided that (i) when the heterocycle contains a nitrogen atom, the nitrogen atom is substituted with R¹⁰ or (ii) the heterocycle does not comprise an —NH— moiety. In some embodiments, R³ is a 7-9 membered heterocycle comprising a fused ring system comprising two rings, wherein the heterocycle includes one or more heteroatoms selected from O, S, and N, and the heterocycle is unsubstituted or is substituted with one or more R¹⁰. In some embodiments, R¹ is a 7-9 membered heterocycle comprising a fused ring system comprising two rings, wherein the heterocycle includes one or more heteroatoms selected from O, S, and N, and the heterocycle is unsubstituted or is substituted with one or more R¹⁰, provided that (i) when the heterocycle contains a nitrogen atom, the nitrogen atom is substituted with R¹⁰ or (ii) the heterocycle does not comprise an —NH— moiety. In some embodiments, R³ comprises a fused ring system comprising two rings, wherein at least one ring is a 5-membered heterocycle that is unsubstituted or is substituted with one or more R¹⁰. In some embodiments, R³ comprises a fused ring system comprising two rings, wherein at least one ring is a pyrrolidine that is unsubstituted or is substituted with one or more R¹⁰. In some embodiments, R³ comprises a fused ring system comprising two rings, wherein each ring is a 5-membered heterocycle that is unsubstituted or is substituted with one or more R¹⁰. In some embodiments, R³ comprises a fused ring system comprising two rings, wherein one ring is a 5-membered heterocycle that is unsubstituted or is substituted with one or more R¹⁰ and the second ring is a 6-membered heterocycle that is unsubstituted or is substituted with one or more R¹⁰. In some embodiments, R¹⁰ comprises a fused ring system comprising two rings, wherein one ring is a pyrrolidine that is unsubstituted or is substituted with one or more R¹⁰ and the second ring is a piperidine, oxazinane, oxazolidine, imidazolidine, or pyrrolidine that is unsubstituted or is substituted with one or more R¹⁰. In some embodiments, the fused ring system is substituted with at least one R¹⁰, wherein the at least one R¹⁰ is selected from ═O and C₁₋₆alkyl, wherein any C₁₋₆alkyl is unsubstituted or substituted with one or more R²⁰.

In some embodiments, when R¹⁰ is —C(O)N(R¹⁴)₂, then at least one R¹⁴ is not H. In some embodiments, when R¹⁰ is —C(O)N(R¹⁴)₂, then each R¹⁰ is C₁₋₄ alkyl (e.g., methyl or ethyl).

In some embodiments, for a compound according to Formula II′ or II-a, R³ is selected from:

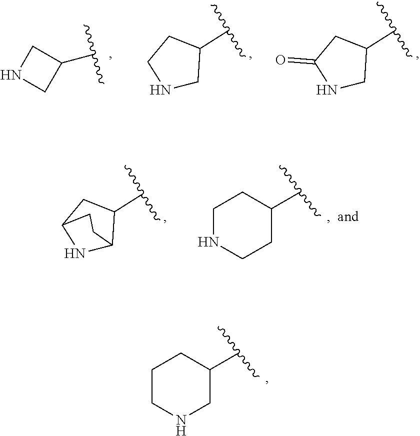 [SMILES: *C1CNC1 ; *C1CCNC1 ; *C1CNC(=O)C1 ; *C1CC2CCC1N2 ; *C1CCNCC1 ; *C1CCCNC1]

any of which is optionally further substituted with one or more R¹⁰.

In some embodiments, for a compound according to any one of Formulas II′, II, and II-a, R³ is selected from:

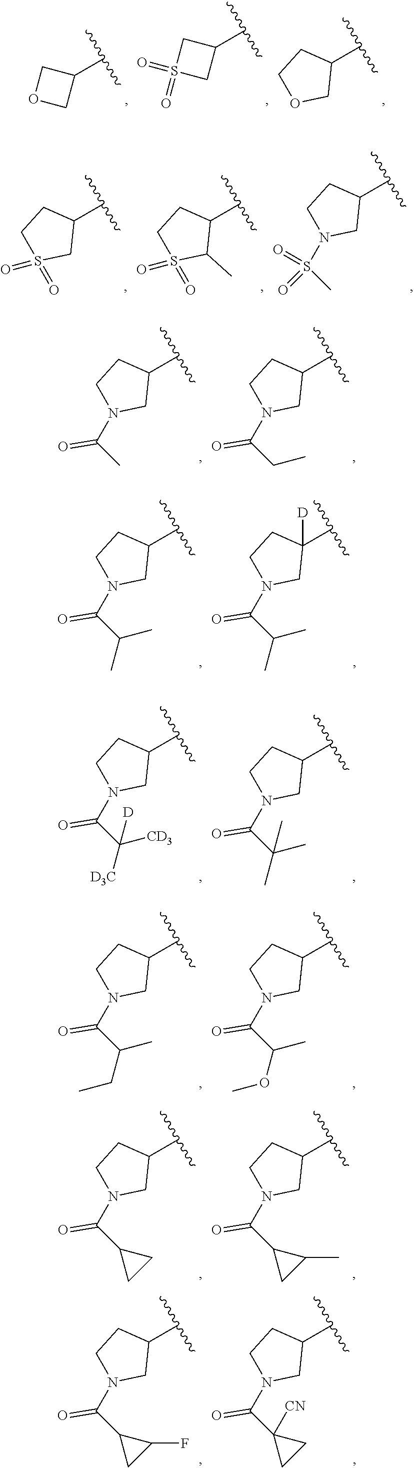

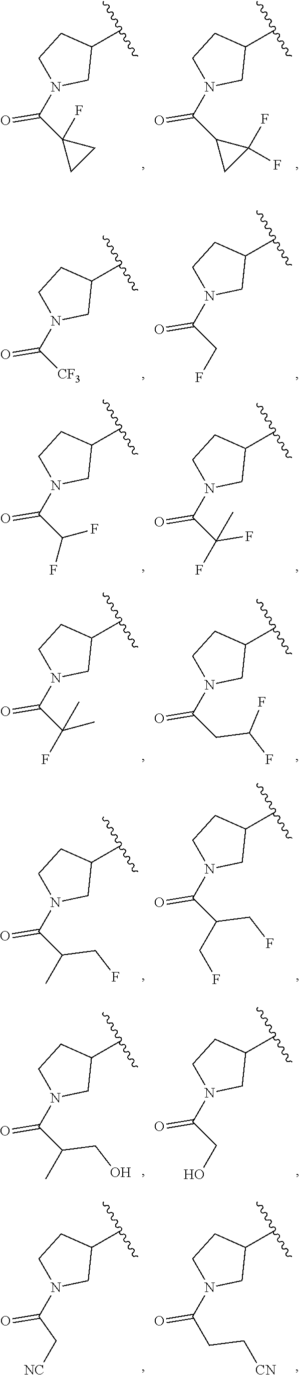

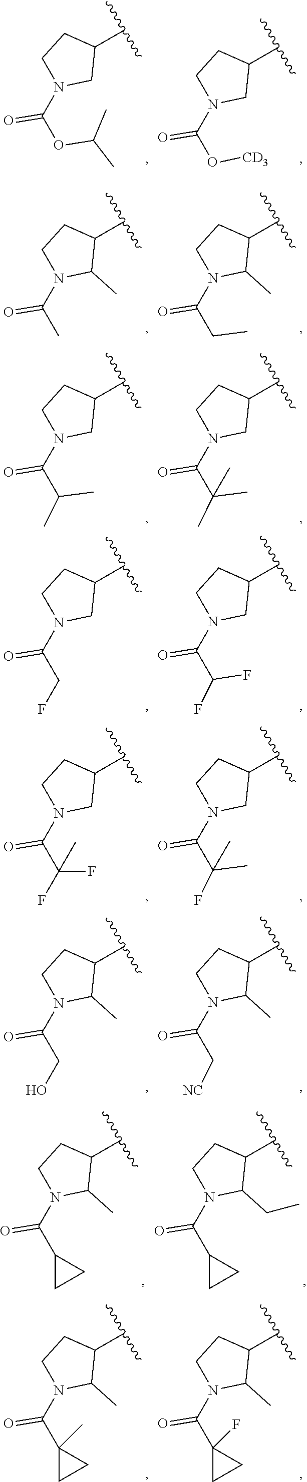

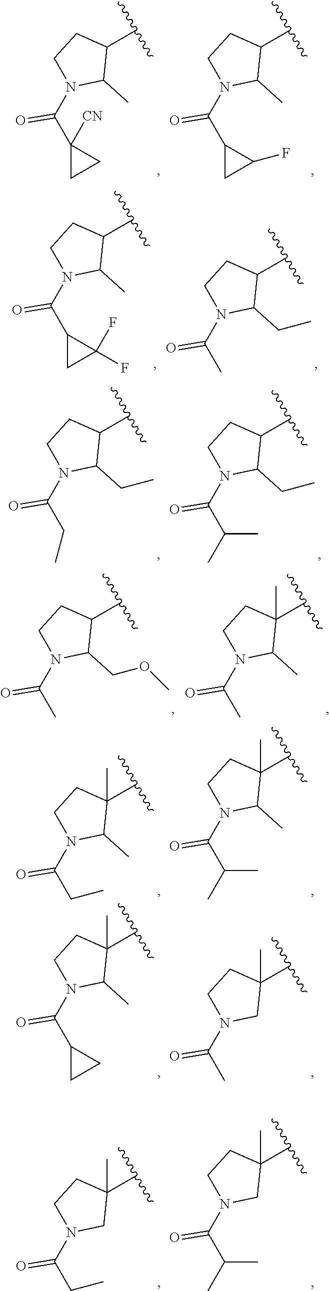

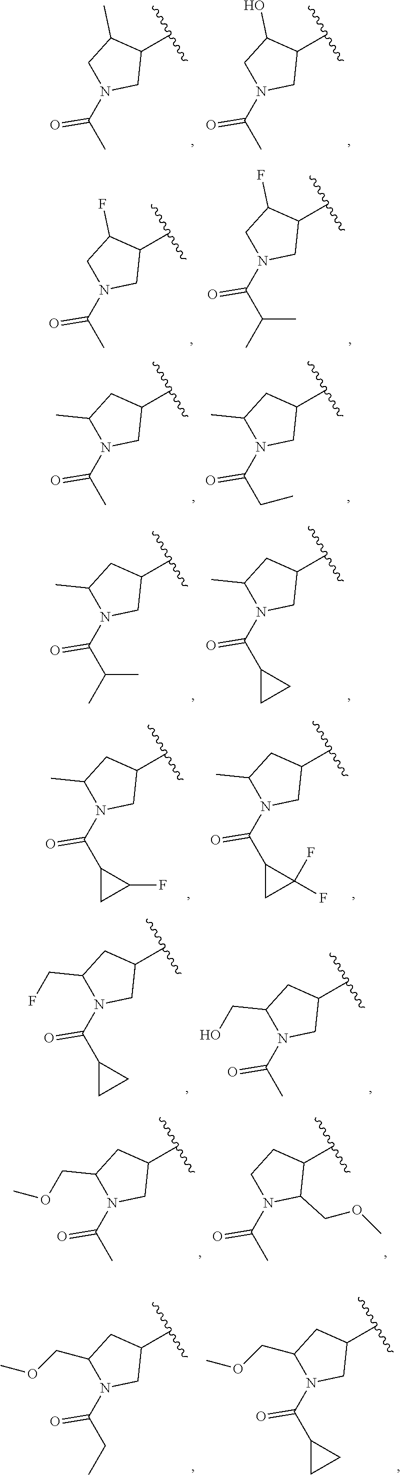

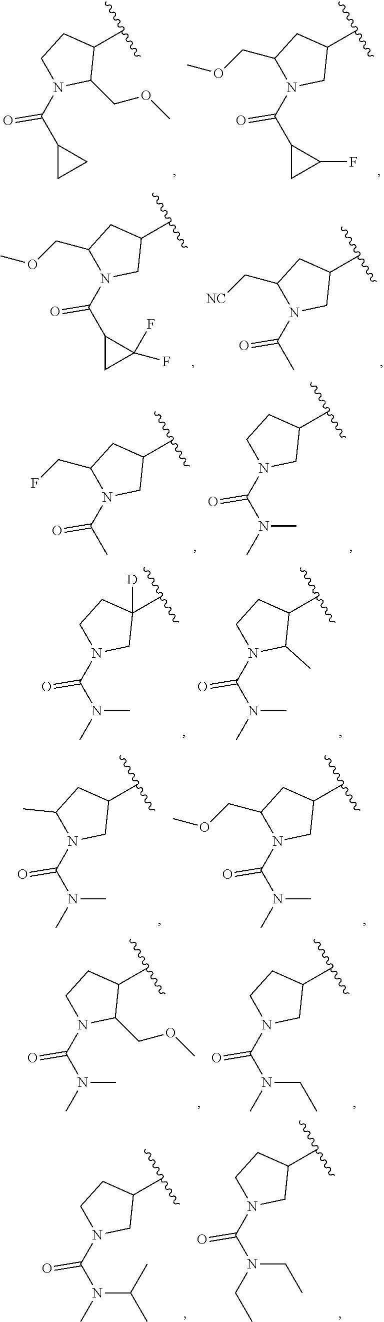

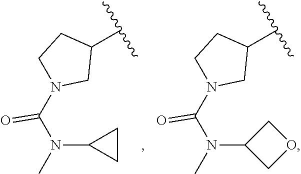

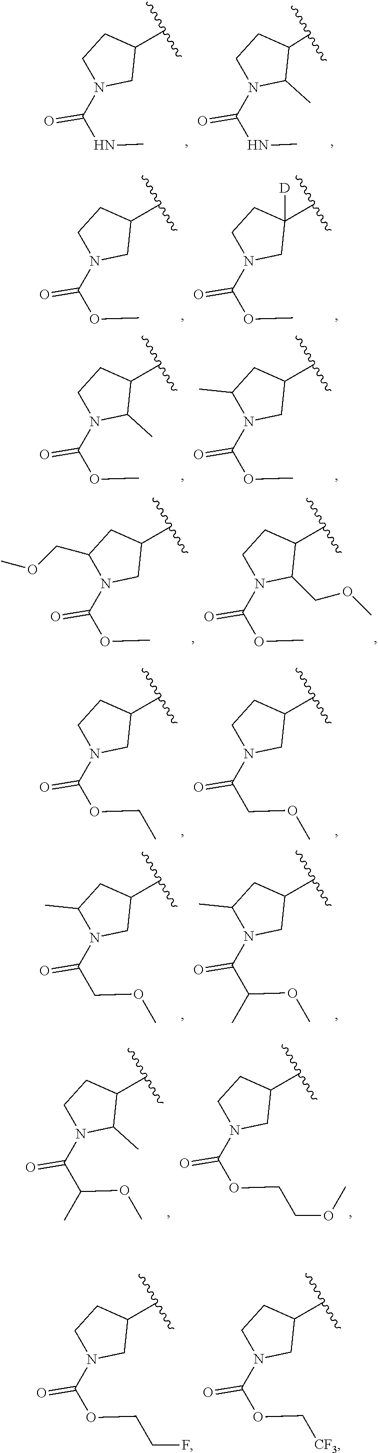

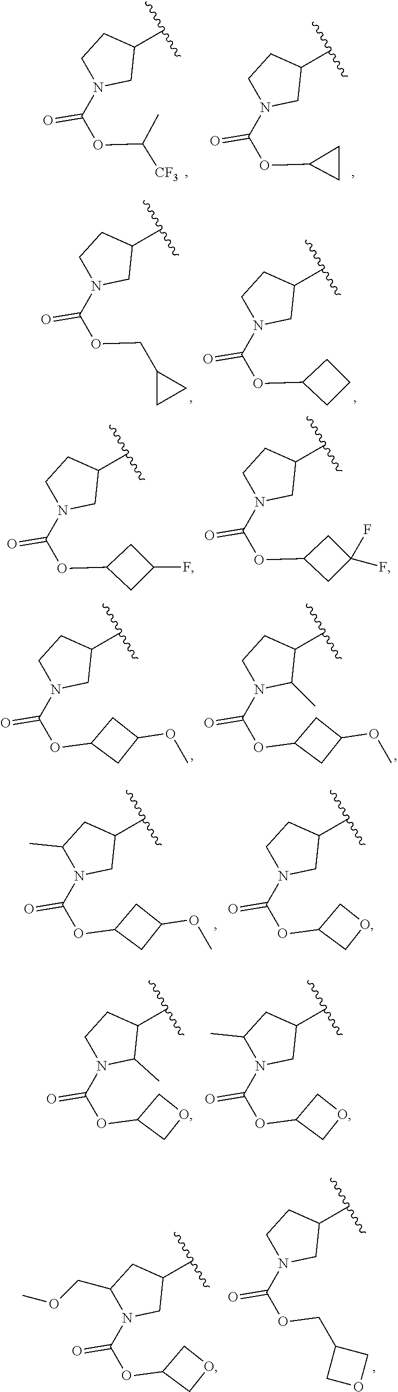

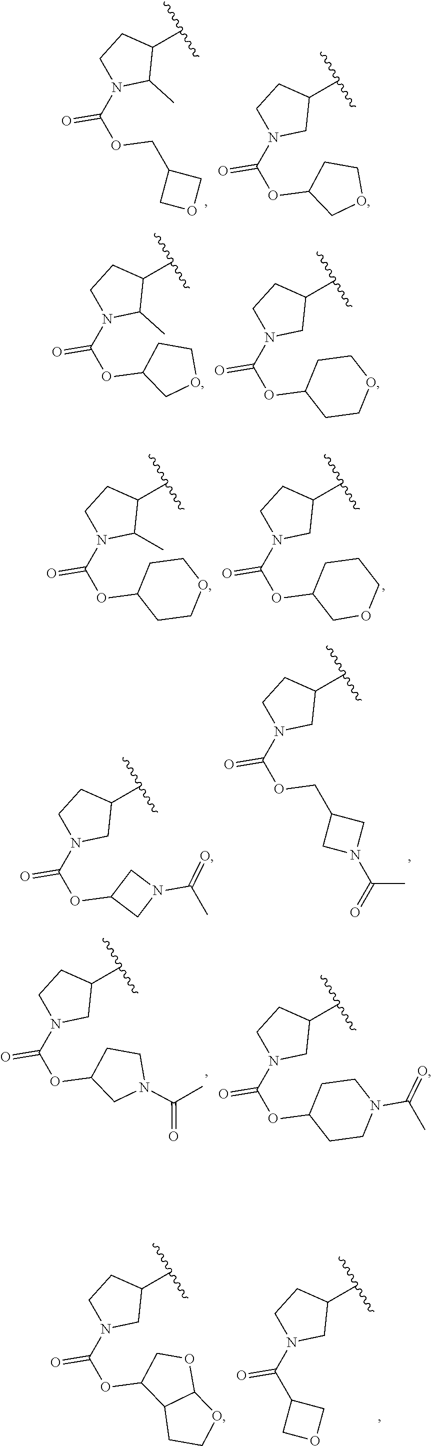

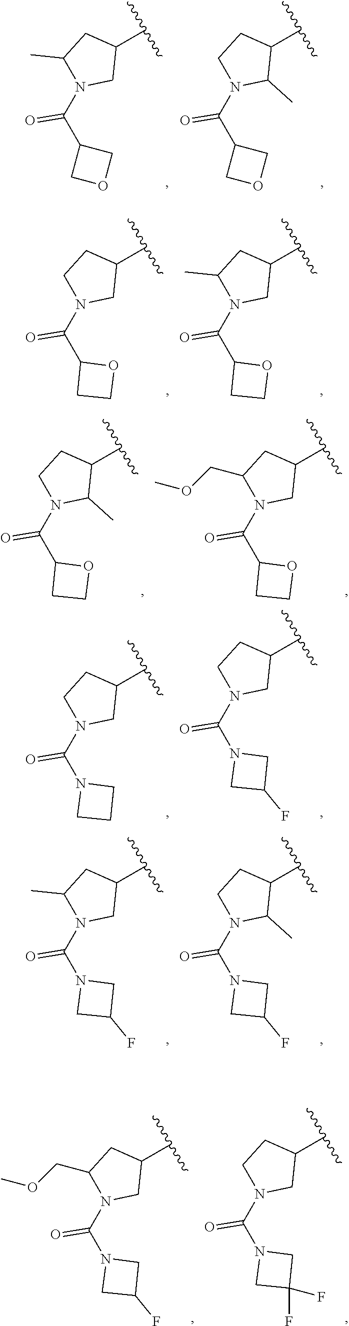

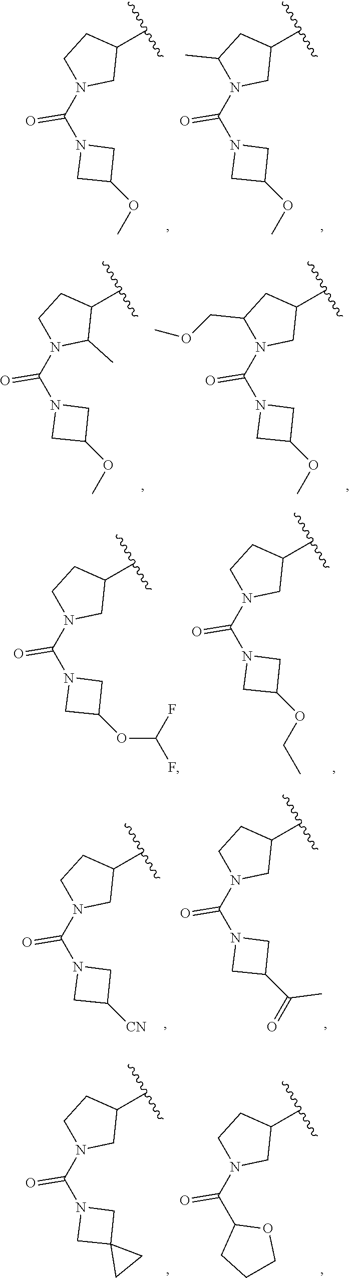

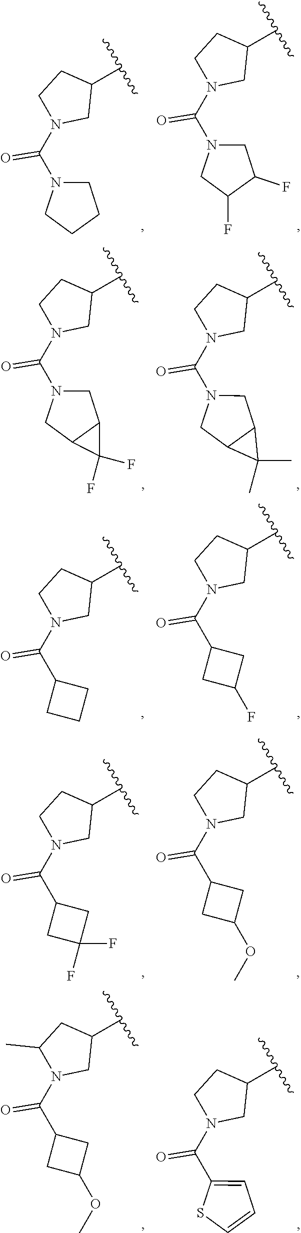

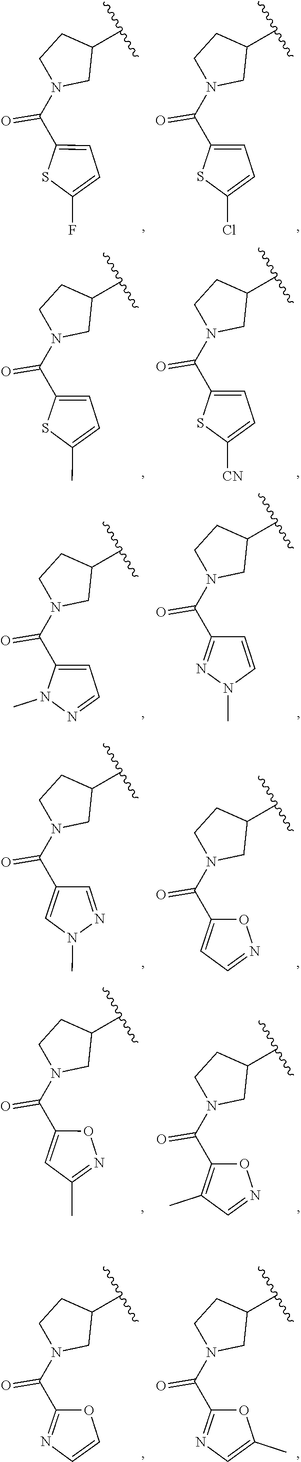

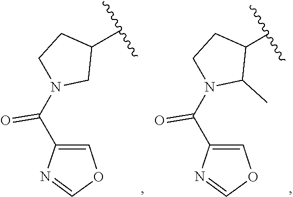 [SMILES: *C1CCN(C(=O)c2cocn2)C1 ; *C1CCN(C(=O)c2cocn2)C1C]

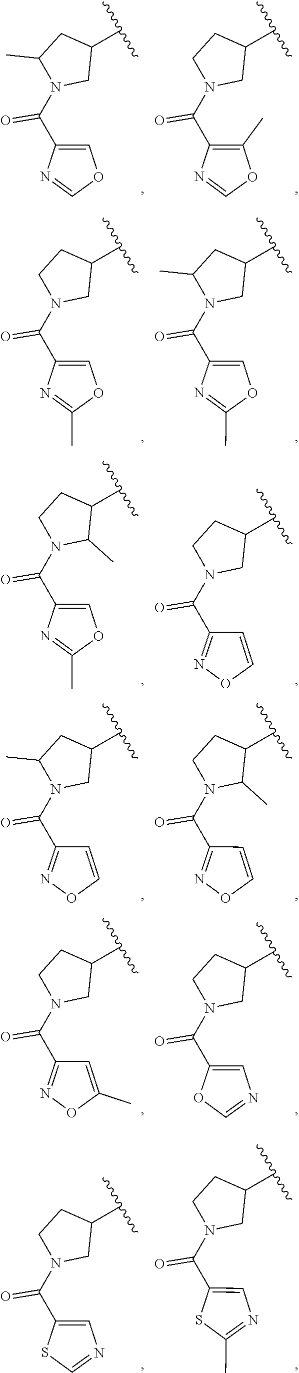

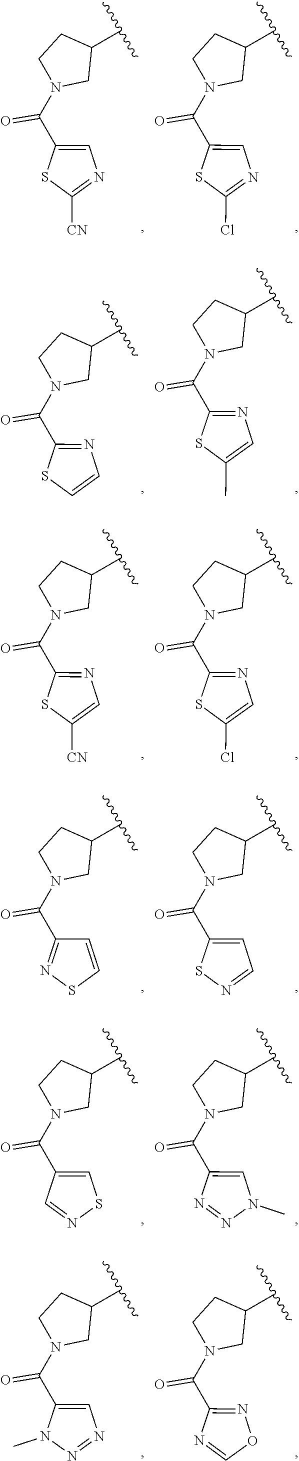

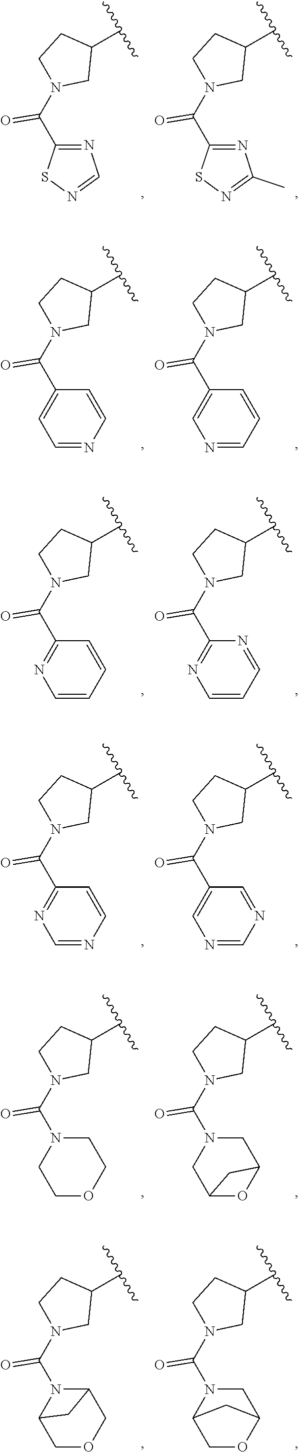

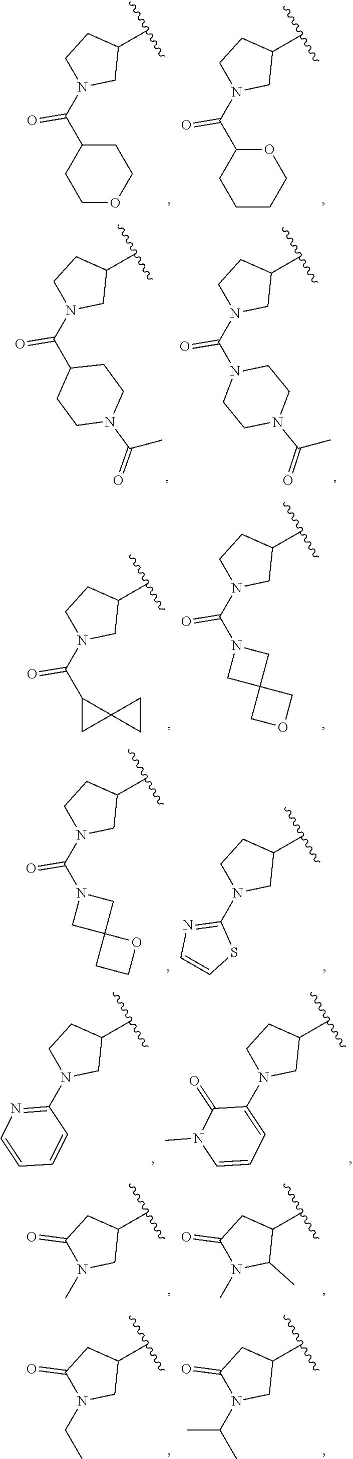

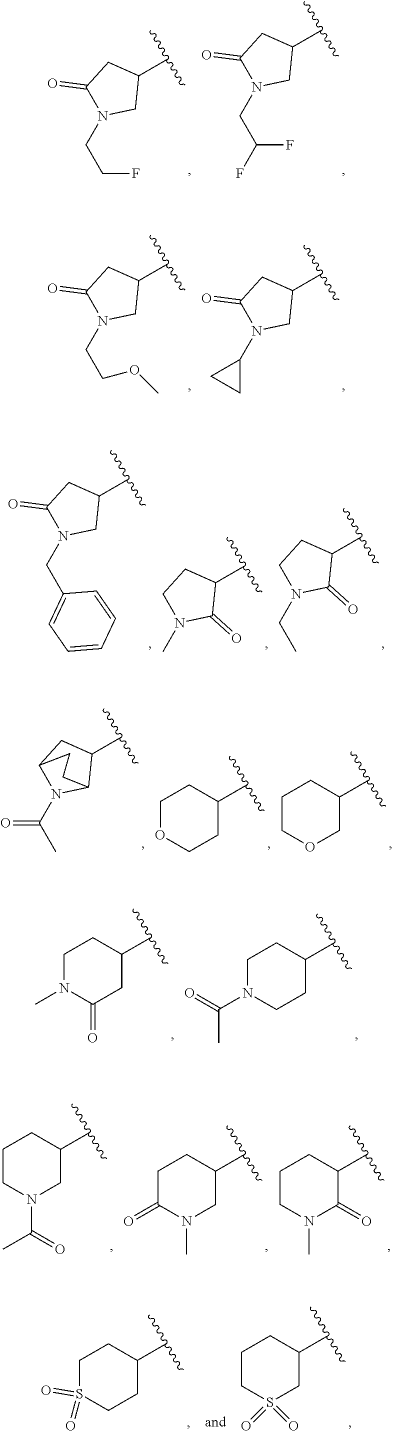

any of which is optionally further substituted with one or more R¹⁰.

In some embodiments, for a compound according to Formula II′ or II, R³ is selected from:

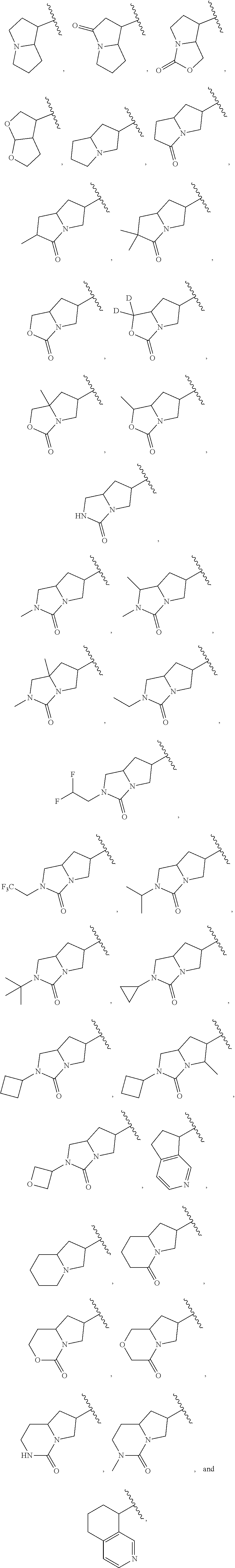

any of which is optionally further substituted with one or more R¹⁰.

In some embodiments, the compound is a compound according to Formula IIA, IIB, IIC, IID, IIE, IIF, IIG, IIH, IIJ, IIK, IIL, IIM, IIN, IIP, IIQ, IIR, IIS, IIT, IIU, IIV, IIW, IIX, IIY, or IIZ:

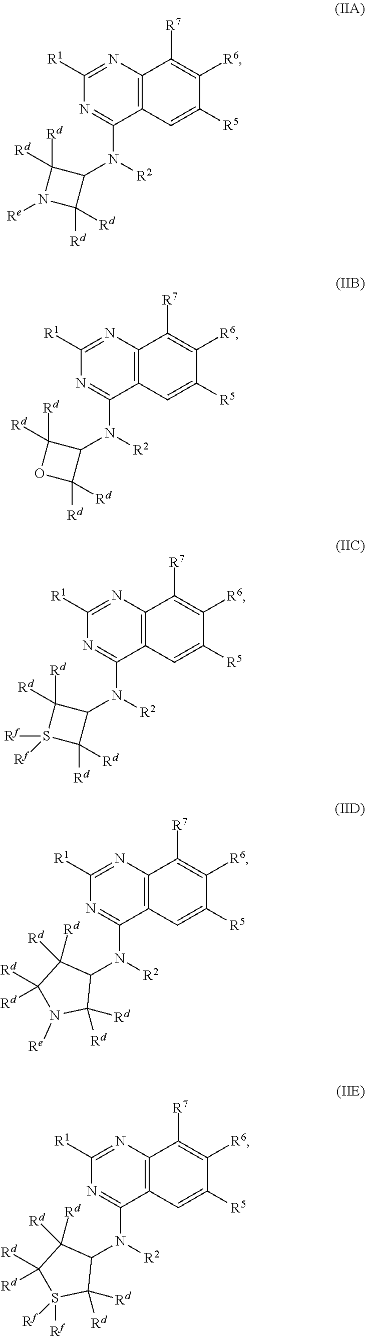

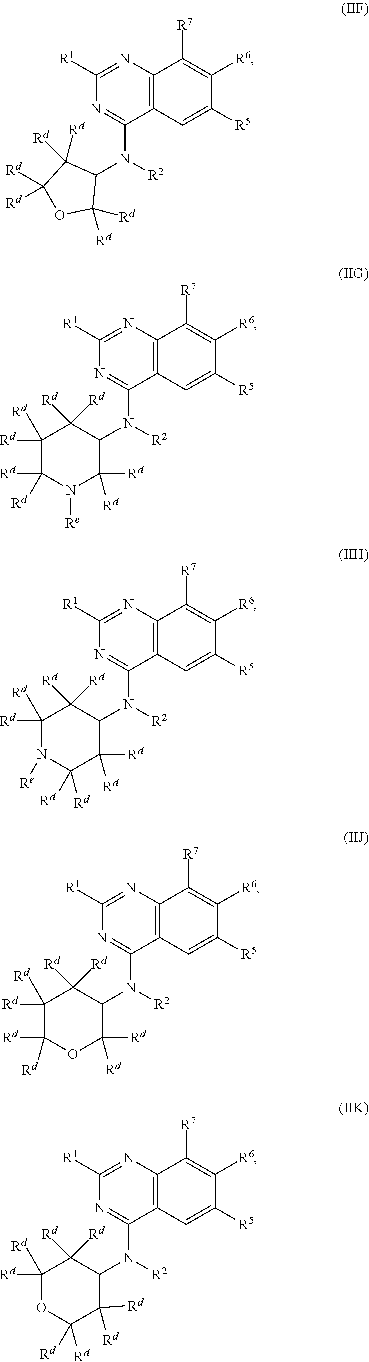

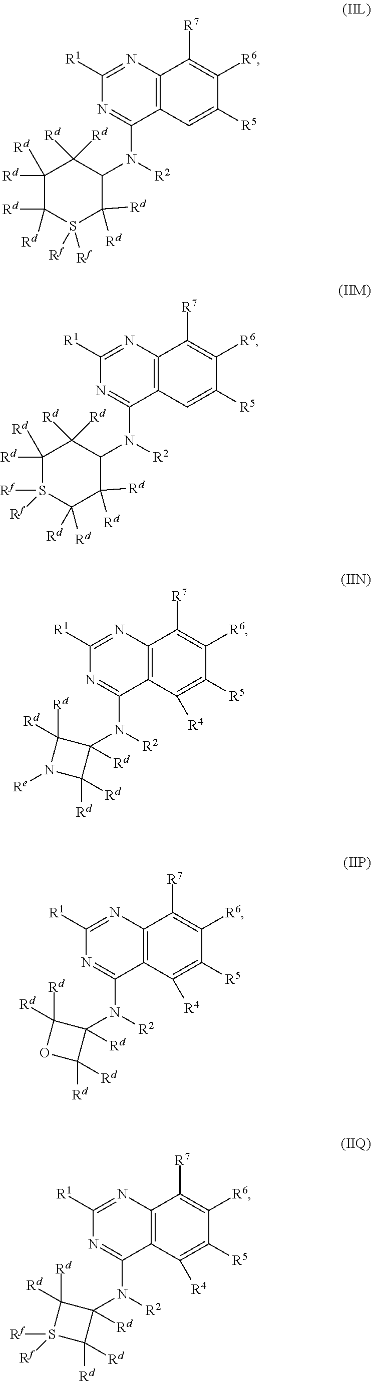

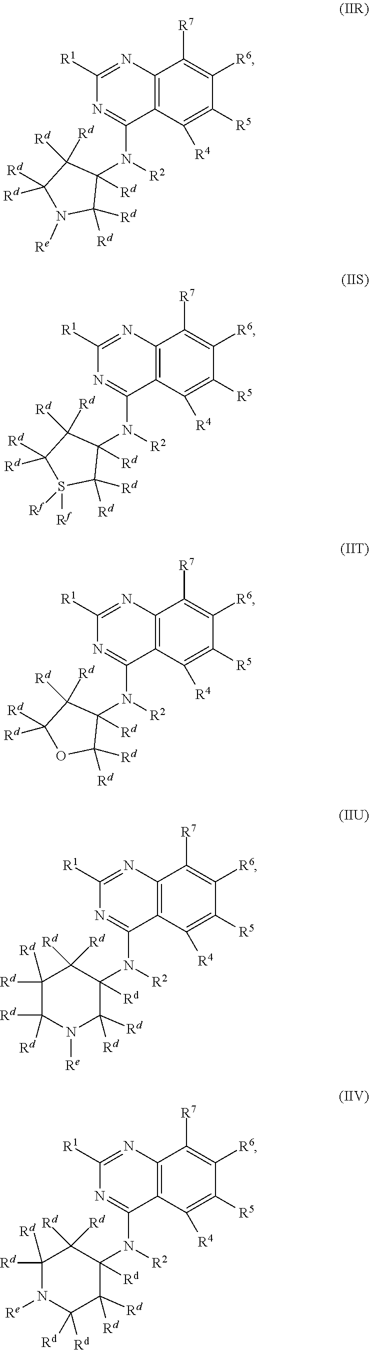

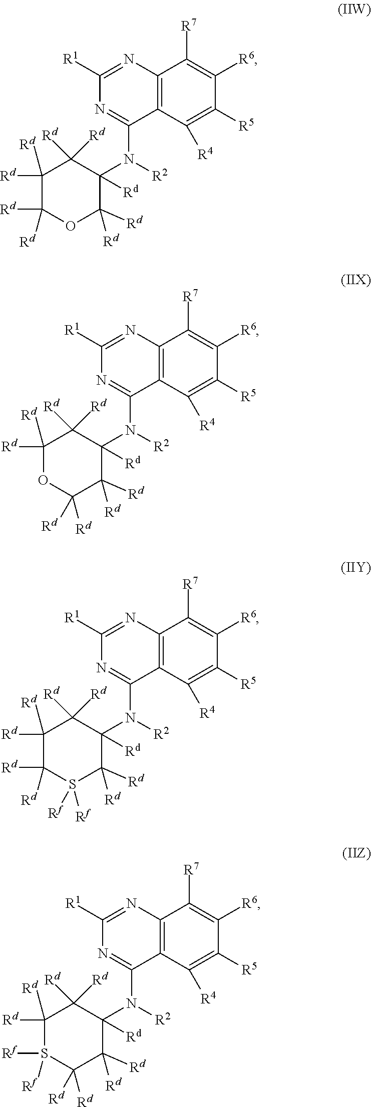

or a salt (e.g., pharmaceutically acceptable salt), ester, tautomer, prodrug, zwitterionic form, or stereoisomer(s) thereof wherein:
R¹ is selected from —OR⁸ and

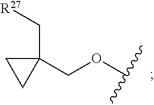

R² is selected from H, C₁₋₆alkyl, and a 3-6 membered carbocycle, wherein any C₁₋₆ alkyl is unsubstituted or is substituted with one or more R¹³;
R⁴, when present, is H;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety ofany alkylheterocycle is selected from C₁₋₆alkyl;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from a 3-6 membered carbocycle, a 3-6 membered heterocycle, C₁₋₆alkyl, C₂₋₄ alkenyl, and H, wherein any C₁₋₆alkyl is optionally deuterated;
each R¹⁵ is independently selected from deuterium, halogen, —N(R¹²)₂, —CN, and C₁₋₆alkyl, wherein any C₁₋₆alkyl is unsubstituted or substituted with one or more R¹³;
each R²⁰ is independently selected from —OH, —OC₁₋₆alkyl, —OC₁₋₆haloalkyl, ═O, —CN, —NH₂, —NHC₁₋₆alkyl, —C(O)(C₁₋₆alkyl), a 3-6 membered carbocycle, phenyl, and halogen;
R²⁷ is a 3-6 membered heterocycle including one or more heteroatoms selected from N, O, and S, wherein the heterocycle is unsubstituted or substituted with one or more R¹³;
each R²⁸ is independently selected from C₁₋₆alkyl and halogen;
R^{a} and R^{b} are each independently selected from deuterium, halogen, C₁₋₆ alkyl, —OR¹², a 3-6 membered carbocycle, and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³;
each R^{d} is independently selected from H, deuterium, —OR¹², ═O, —C(O)(C₁₋₆alkylene)CN, —C(O)(C₁₋₆alkylene)OH, —C(O)(C₁₋₆alkyl), —C(O)(3-6 membered carbocycle), —S(O)₂(C₁₋₆alkyl), halogen, and C₁₋₆alkyl, wherein any C₁₋₆alkyl is unsubstituted or substituted with one or more R²⁰, and wherein any 3-6 membered carbocycle is unsubstituted or substituted with one or more R¹² or R²⁰;
R^{e}, if present, is selected from —C(O)(C₁₋₆alkylene)CN, —C(O)(C₁₋₆alkylene)OH, —C(O)(C₁₋₆alkyl), —C(O)O(C₁₋₆alkyl), —C(O)OR¹⁴, —C(O)(3-6 membered carbocycle), —C(O)(3-8 membered heterocycle), —C(O)(5-6 membered heteraryl), —C(O)O(3-6 membered carbocycle), —C(O)O(3-6 membered heterocycle), —C(O)O(C₁₋₆alkylene)(3-6 membered heterocycle), —C(O)N(R¹⁴)₂, —S(O)₂(C₁₋₆alkyl), a 5-6 membered heteroaryl, a 3-6 membered carbocycle, and C₁₋₆alkyl, wherein any C₁₋₆alkyl is optionally deuterated and is unsubstituted or substituted with one or more R¹⁰, and wherein any 3-6 membered carbocycle, 5-6 membered heteroaryl, or 3-6 or 3-8 membered heterocycle is unsubstituted or substituted with one or more R¹² or R²⁰; and
each R^{f} is optionally absent and, if present, is selected from ═O, —NH₂, —NHC₁₋₆alkyl, and —N(C₁₋₆alkyl)₂.

In some embodiments, the compound is a compound according to Formula IIA, or a salt (e.g., pharmaceutically acceptable salt), ester, tautomer, prodrug, zwitterionic form, or stereoisomer(s) thereof. In some embodiments, the compound is a compound according to Formula TIB, or a salt (e.g., pharmaceutically acceptable salt), ester, tautomer, prodrug, zwitterionic form, or stereoisomer(s) thereof. In some embodiments, the compound is a compound according to Formula IIC, or a salt (e.g., pharmaceutically acceptable salt), ester, tautomer, prodrug, zwitterionic form, or stereoisomer(s) thereof. In some embodiments, the compound is a compound according to Formula IID, or a salt (e.g., pharmaceutically acceptable salt), ester, tautomer, prodrug, zwitterionic form, or stereoisomer(s) thereof. In some embodiments, the compound is a compound according to Formula IIE, or a salt (e.g., pharmaceutically acceptable salt), ester, tautomer, prodrug, zwitterionic form, or stereoisomer(s) thereof. In some embodiments, the compound is a compound according to Formula IIF, or a salt (e.g., pharmaceutically acceptable salt), ester, tautomer, prodrug, zwitterionic form, or stereoisomer(s) thereof. In some embodiments, the compound is a compound according to Formula IIG, or a salt (e.g., pharmaceutically acceptable salt), ester, tautomer, prodrug, zwitterionic form, or stereoisomer(s) thereof. In some embodiments, the compound is a compound according to Formula IIH, or a salt (e.g., pharmaceutically acceptable salt), ester, tautomer, prodrug, zwitterionic form, or stereoisomer(s) thereof. In some embodiments, the compound is a compound according to Formula IIJ, or a salt (e.g., pharmaceutically acceptable salt), ester, tautomer, prodrug, zwitterionic form, or stereoisomer(s) thereof. In some embodiments, the compound is a compound according to Formula IIK, or a salt (e.g., pharmaceutically acceptable salt), ester, tautomer, prodrug, zwitterionic form, or stereoisomer(s) thereof. In some embodiments, the compound is a compound according to Formula IIL, or a salt (e.g., pharmaceutically acceptable salt), ester, tautomer, prodrug, zwitterionic form, or stereoisomer(s) thereof. In some embodiments, the compound is a compound according to Formula IIM, or a salt (e.g., pharmaceutically acceptable salt), ester, tautomer, prodrug, zwitterionic form, or stereoisomer(s) thereof. In some embodiments, the compound is a compound according to Formula IIN, or a salt (e.g., pharmaceutically acceptable salt), ester, tautomer, prodrug, zwitterionic form, or stereoisomer(s) thereof. In some embodiments, the compound is a compound according to Formula IIP, or a salt (e.g., pharmaceutically acceptable salt), ester, tautomer, prodrug, zwitterionic form, or stereoisomer(s) thereof. In some embodiments, the compound is a compound according to Formula IIQ, or a salt (e.g., pharmaceutically acceptable salt), ester, tautomer, prodrug, zwitterionic form, or stereoisomer(s) thereof. In some embodiments, the compound is a compound according to Formula IIR or a salt (e.g., pharmaceutically acceptable salt), ester, tautomer, prodrug, zwitterionic form, or stereoisomer(s) thereof. In some embodiments, the compound is a compound according to Formula IIS, or a salt (e.g., pharmaceutically acceptable salt), ester, tautomer, prodrug, zwitterionic form, or stereoisomer(s) thereof. In some embodiments, the compound is a compound according to Formula IIT, or a salt (e.g., pharmaceutically acceptable salt), ester, tautomer, prodrug, zwitterionic form, or stereoisomer(s) thereof. In some embodiments, the compound is a compound according to Formula IIU, or a salt (e.g., pharmaceutically acceptable salt), ester, tautomer, prodrug, zwitterionic form, or stereoisomer(s) thereof. In some embodiments, the compound is a compound according to Formula IIV, or a salt (e.g., pharmaceutically acceptable salt), ester, tautomer, prodrug, zwitterionic form, or stereoisomer(s) thereof. In some embodiments, the compound is a compound according to Formula IIW, or a salt (e.g., pharmaceutically acceptable salt), ester, tautomer, prodrug, zwitterionic form, or stereoisomer(s) thereof. In some embodiments, the compound is a compound according to Formula IIX, or a salt (e.g., pharmaceutically acceptable salt), ester, tautomer, prodrug, zwitterionic form, or stereoisomer(s) thereof. In some embodiments, the compound is a compound according to Formula IIY, or a salt (e.g., pharmaceutically acceptable salt), ester, tautomer, prodrug, zwitterionic form, or stereoisomer(s) thereof. In some embodiments, the compound is a compound according to Formula IIZ, or a salt (e.g., pharmaceutically acceptable salt), ester, tautomer, prodrug, zwitterionic form, or stereoisomer(s) thereof. In some embodiments, the compound is a compound according to Formula IIA, or a salt (e.g., pharmaceutically acceptable salt) thereof. In some embodiments, the compound is a compound according to Formula IIB, or a salt (e.g., pharmaceutically acceptable salt) thereof. In some embodiments, the compound is a compound according to Formula IIC, or a salt (e.g., pharmaceutically acceptable salt) thereof. In some embodiments, the compound is a compound according to Formula ID, or a salt (e.g., pharmaceutically acceptable salt) thereof. In some embodiments, the compound is a compound according to Formula IIE, or a salt (e.g., pharmaceutically acceptable salt) thereof. In some embodiments, the compound is a compound according to Formula IIF, or a salt (e.g., pharmaceutically acceptable salt) thereof. In some embodiments, the compound is a compound according to Formula IIG, or a salt (e.g., pharmaceutically acceptable salt) thereof. In some embodiments, the compound is a compound according to Formula IIH, or a salt (e.g., pharmaceutically acceptable salt) thereof. In some embodiments, the compound is a compound according to Formula IIJ, or a salt (e.g., pharmaceutically acceptable salt) thereof. In some embodiments, the compound is a compound according to Formula IIK, or a salt (e.g., pharmaceutically acceptable salt) thereof. In some embodiments, the compound is a compound according to Formula IIL, or a salt (e.g., pharmaceutically acceptable salt) thereof. In some embodiments, the compound is a compound according to Formula IIM, or a salt (e.g., pharmaceutically acceptable salt) thereof. In some embodiments, the compound is a compound according to Formula IN, or a salt (e.g., pharmaceutically acceptable salt) thereof. In some embodiments, the compound is a compound according to Formula IIP, or a salt (e.g., pharmaceutically acceptable salt) thereof. In some embodiments, the compound is a compound according to Formula IIQ, or a salt (e.g., pharmaceutically acceptable salt) thereof. In some embodiments, the compound is a compound according to Formula IIR, or a salt (e.g., pharmaceutically acceptable salt) thereof. In some embodiments, the compound is a compound according to Formula IIS, or a salt (e.g., pharmaceutically acceptable salt) thereof. In some embodiments, the compound is a compound according to Formula IIT, or a salt (e.g., pharmaceutically acceptable salt) thereof. In some embodiments, the compound is a compound according to Formula IIU, or a salt (e.g., pharmaceutically acceptable salt) thereof. In some embodiments, the compound is a compound according to Formula IIV, or a salt (e.g., pharmaceutically acceptable salt) thereof. In some embodiments, the compound is a compound according to Formula IIW, or a salt (e.g., pharmaceutically acceptable salt) thereof. In some embodiments, the compound is a compound according to Formula IIX, or a salt (e.g., pharmaceutically acceptable salt) thereof. In some embodiments, the compound is a compound according to Formula IIY, or a salt (e.g., pharmaceutically acceptable salt) thereof. In some embodiments, the compound is a compound according to Formula IIZ, or a salt (e.g., pharmaceutically acceptable salt) thereof.

In some embodiments, the compound is a compound according to Formula IIAA:

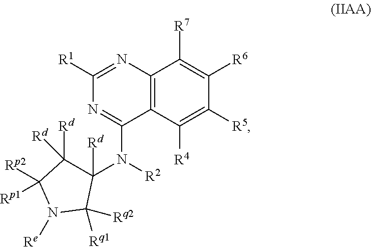

or a salt (e.g., pharmaceutically acceptable salt), ester, tautomer, prodrug, zwitterionic form, or stereoisomer(s) thereof, wherein:

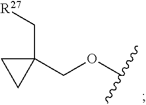

R¹ is selected from —OR⁸ and;
R² is selected from H, C₁₋₆alkyl, and a 3-6 membered carbocycle, wherein any C₁₋₆ alkyl is unsubstituted or is substituted with one or more R¹³;
R⁴ is H;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹² is independently selected from C₁₋₆alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹²)₂, and halogen;
each R¹⁴ is independently selected from a 3-6 membered carbocycle, a 3-6 membered heterocycle, C₁₋₆ alkyl, C₂₋₆alkenyl, and H, wherein any C₁₋₆alkyl is optionally deuterated;
each R¹⁵ is independently selected from deuterium, halogen, —N(R¹²)₂, —CN, and C₁₋₆alkyl, wherein any C₁₋₆alkyl is unsubstituted or substituted with one or more R¹³;
each R²⁰ is independently selected from —OH, —OC₁₋₆alkyl, —OC₁₋₆haloalkyl, ═O, —CN, —NH₂, —NHC₁₋₆alkyl, —C(O)(C₁₋₆alkyl), a 3-6 membered carbocycle, phenyl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen;
R^{a} and R^{b} are each independently selected from deuterium, halogen, C₁₋₆ alkyl, —OR², a 3-6 membered carbocycle, and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³:
each R^{d} is independently selected from H, deuterium, —OR¹², ═O, —C(O)(C₁₋₆alkylene)CN, —C(O)(C₁₋₆alkylene)OH, —C(O)(C₁₋₆alkyl), —C(O)(3-6 membered carbocycle), —S(O)₂(C₁₋₆alkyl), halogen, a 3-6 membered carbocycle, a 3-6 membered heterocycle, and C₁₋₆alkyl, wherein any C₁₋₆alkyl is unsubstituted or substituted with one or more R²⁰, wherein any 3-6 membered carbocycle or 3-6 membered heterocycle, is unsubstituted or substituted with one or more R¹² or R²⁰; and
(i) R^{q1}, R^{q2}, and R^{p2} are each independently selected from R^{d}, and R^{c} and R^{p1}, together with the atoms to which they are attached, form a 5-6 membered heterocycle that is unsubstituted or is substituted with one or more R^{d}; or
(ii) R^{p1}, R^{p2}, and R^{q2} are each independently selected from R^{d}, and R^{c} and R^{q1}, together with the atoms to which they are attached, form a 5-6 membered heterocycle that is unsubstituted or is substituted with one or more R^{d}.

In some embodiments, the compound is a compound according to Formula IIAA, or a salt (e.g., pharmaceutically acceptable salt) thereof.

In some embodiments, for a compound according to any one of Formulas I, II′, II-a, UA, IIB, IIC, IID, IIE, IIF, IIG, IIH, IIJ, IIK, IIL, IIM, IIN, IIP, IIQ, IIR, IIS, IIT, IIU, IIV, IIW, IIX, IIY, IIZ, and IIAA, R⁶ is a monocyclic heteroaryl that is substituted with one or more R¹⁵. In some embodiments, R⁶ is a pyridine substituted with one or more R¹⁵. In some embodiments, at least one R¹⁵ is selected from C₁₋₆alkyl, wherein any C₁₋₆alkyl is unsubstituted or substituted with one or more R¹³. In some embodiments, at least one R¹⁵ is —N(R¹²)₂. In some embodiments, R⁶ is selected from

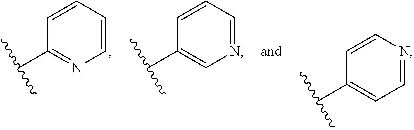

any of which is optionally substituted with one or more R¹⁵. In some embodiments, R⁶ is

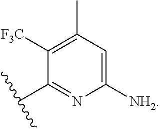 [SMILES: *c1nc(N)cc(C)c1C(F)(F)F]

In some embodiments, for a compound according to any one of Formulas II, II′, II-a, IIA, IIB, IIC, IID, IIE, IIF, IIG, IIH, IIJ, IIK, IIL, IIM, IIN, IIP, IIQ, IIR, IIS, IIT, IIU, IIV, IIW, IIX, HY, IIZ, and IIAA, R⁶ is a bicyclic heteroaryl that is substituted with one or more R's. In some embodiments, R¹ is selected from:

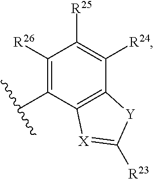

wherein X is selected from N and C—CN; Y is selected from O and S; R²³ is selected from —N(R¹²)₂, C₁₋₆ alkyl, and C₁₋₆alkyl-N(R¹²)₂, wherein any C₁₋₆alkyl is unsubstituted or substituted with one or more R¹³; and R²⁴, R²⁵, and R²⁶ are independently selected from H, deuterium, halogen, and C₁₋₆alkyl, wherein any C₁₋₆alkyl is unsubstituted or substituted with one or more R¹³. In some embodiments, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R¹²)₂. In some embodiments, X is C—CN, Y is S, and R²³ is —NH₂. In some embodiments, R²⁴ is a halogen (e.g., fluoro). In some embodiments, R²⁶ is deuterium.

In some embodiments, for a compound according to any one of Formulas II, II′, II-a, IIA, IIB, IIC, IID, IIE, IIF, IIG, IIH, IIJ, IIK, IIL, IIM, IIN, IIP, IIQ, IIR, IIS, IIT, IIU, IIV, IIW, IIX, IIY, IIZ, and IIAA, R⁶ is selected from:

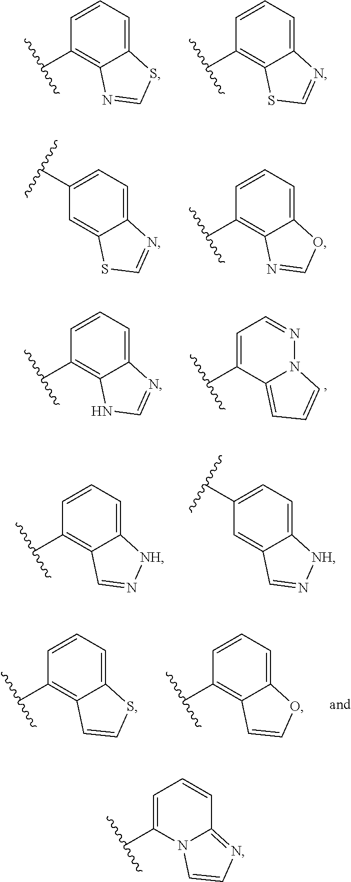

any of which is substituted with one or more R¹³.

In some embodiments, for a compound according to any one of Formulas II, II′, II-a, IIA, IIB, IIC, IID, IIE, IIF, IIG, IIH, IIJ, IIK, IIL, IIM, IIN, IIP, IIQ, IIR, IIS, IIT, IIU, IIV, IIW, IIX, IIY, IIZ, and IIAA, R⁶ is selected from:

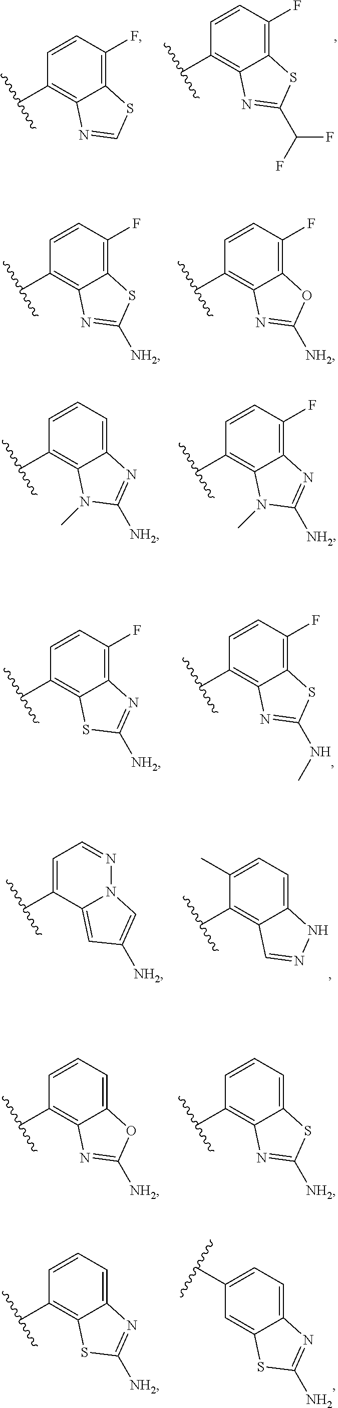

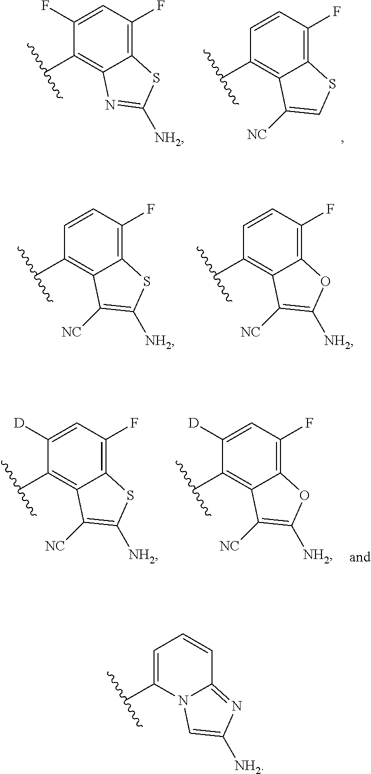

In some embodiments, for a compound according to any one of Formulas II, II′, II-a, IIA, IIB, IIC, IID, IIE, IIF, IIG, IIH, IIJ, IIK, IIL, IIM, IIN, IIP, IIQ, IIR, IIS, IIT, IIU, IIV, IIW, IIX, IIY, IIZ, and IIAA, R⁶ is selected from:

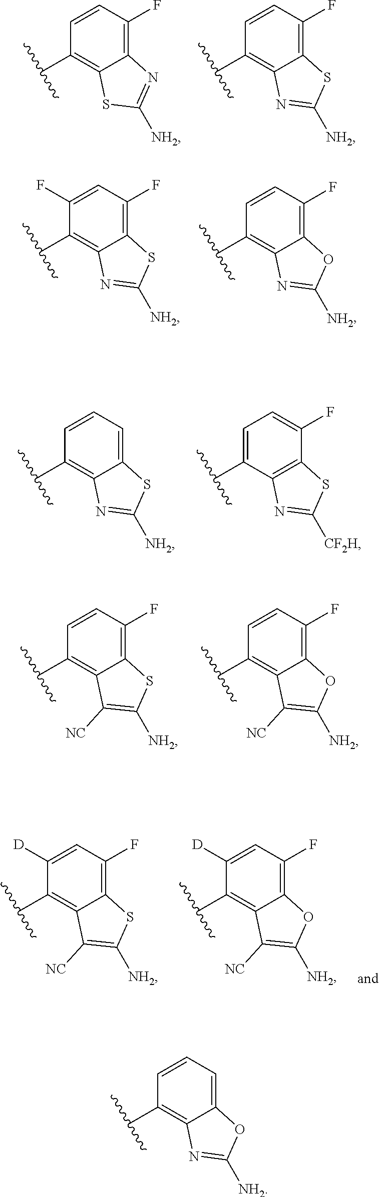

In some embodiments, for a compound according to any one of Formulas I, II′, II-a, IIA, IIB, IIC, IID, IIE, IIF, IIG, IIH, IIJ, IIK, IIL, IIM, IIN, II, IIQ, IIR, IIS, IIT, IIU, IIV, IIW, IIX, IIY, IIZ, and IIAA, R⁶ is

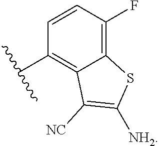 [SMILES: *c1ccc(F)c2sc(N)c(C#N)c12]

In some embodiments, the compound is a compound according to Formula IIA1, IIB1, IIC1, ID1, IIE1, IIF1, IIG1, IIH1, IIU1, IIK1, IIL1, IIM1, IIN1, IIP1, IIQ1, IIR1, IIS1, IIT1, IIU1, IIV1, IIW1, IIX1, IIY1, or IIZ1:

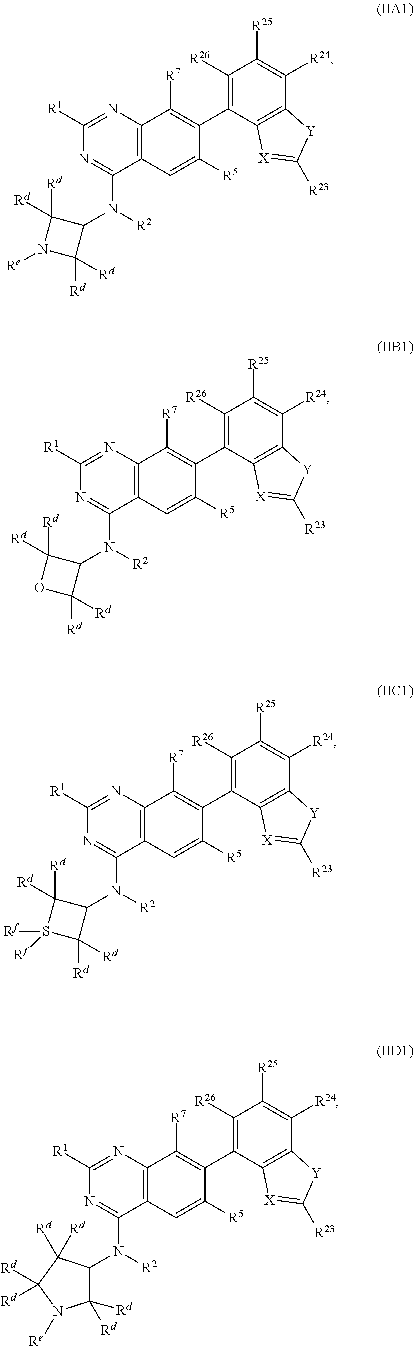

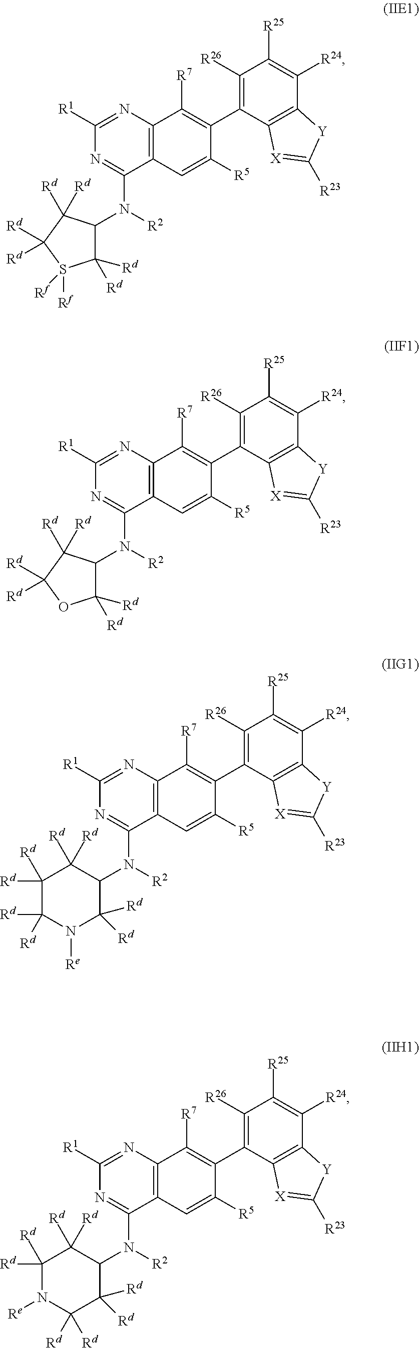

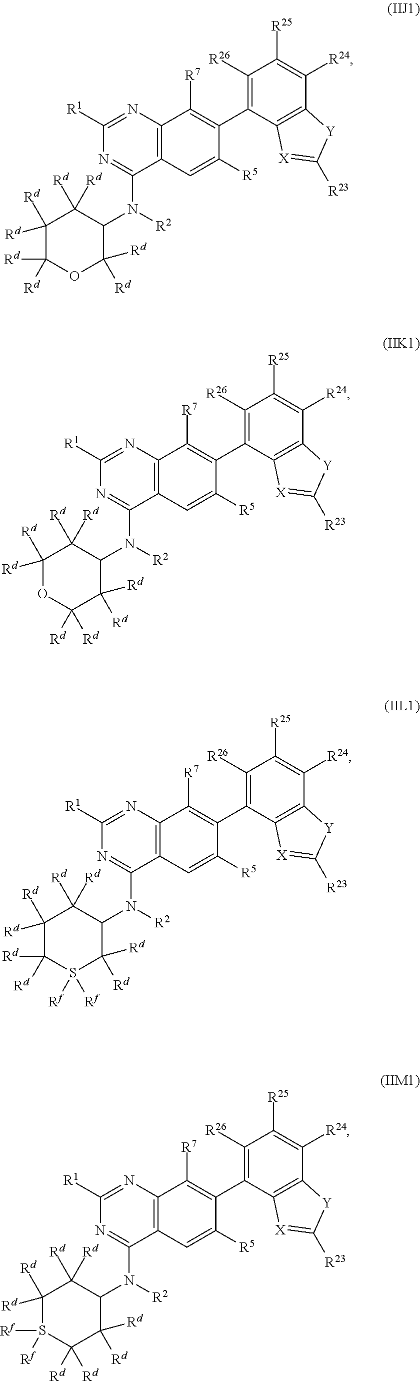

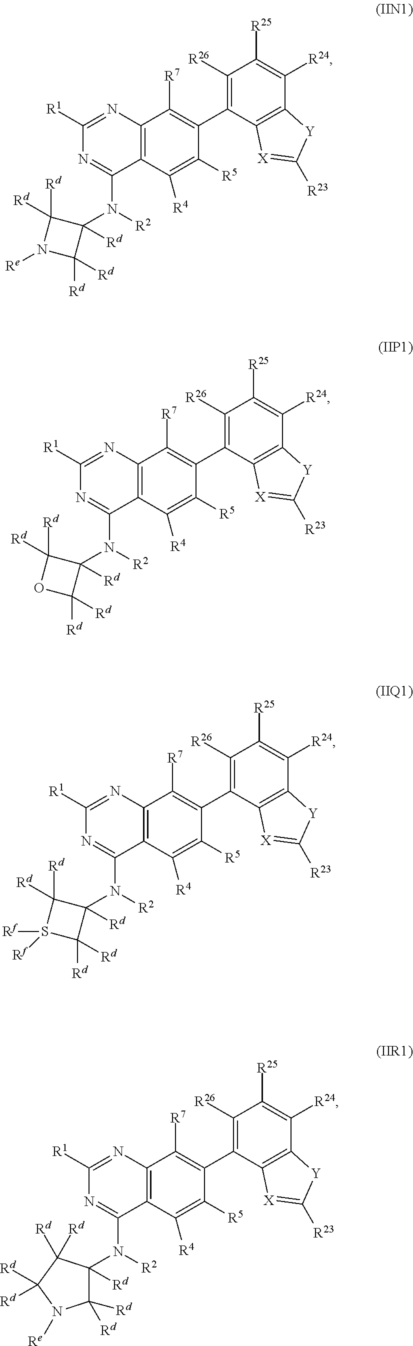

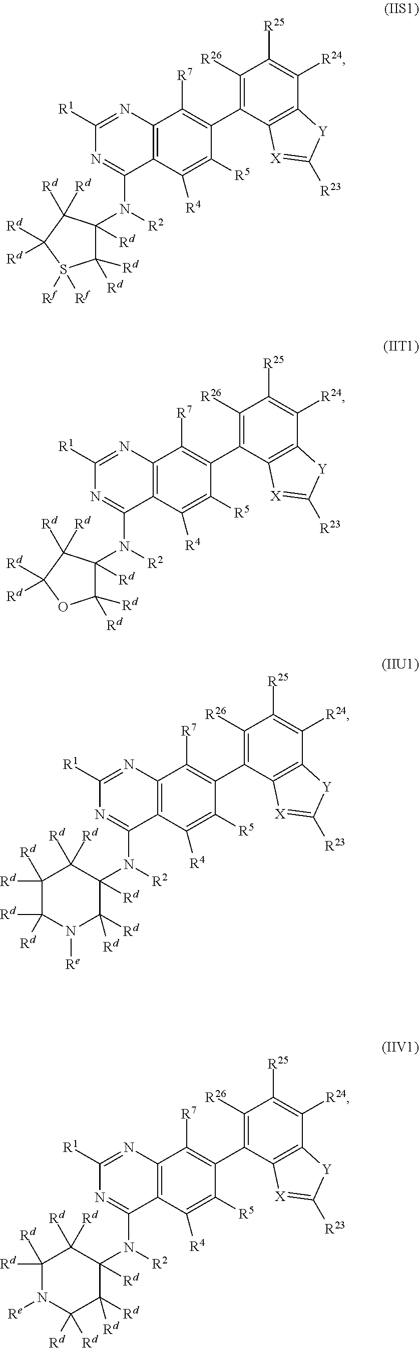

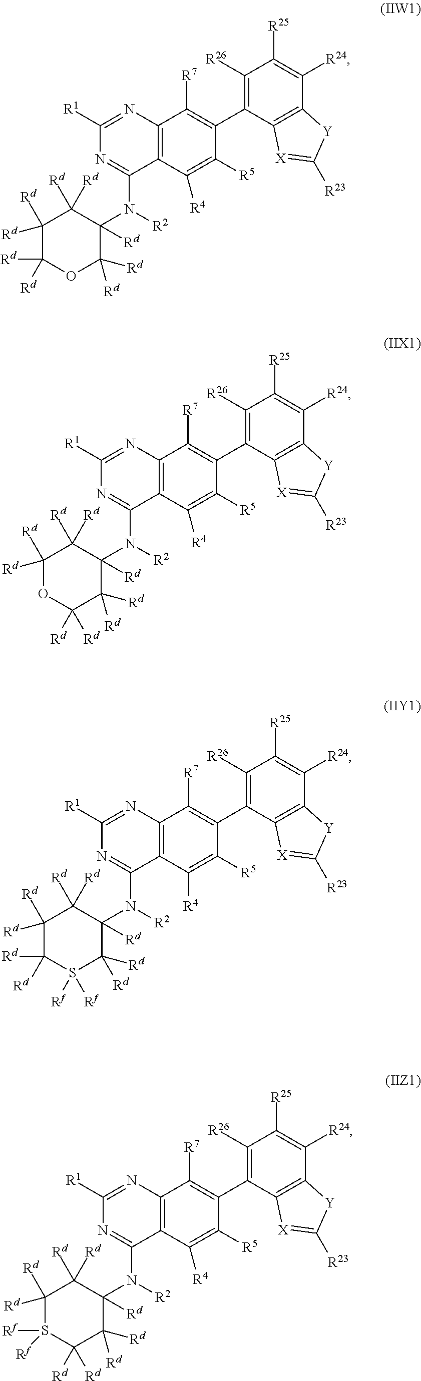

or a salt (e.g., pharmaceutically acceptable salt), ester, tautomer, prodrug, zwitterionic form, or stereoisomer(s) thereof, wherein:

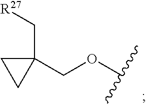

R¹ is selected from —OR⁸ and;
R² is selected from H, C₁₋₆alkyl, and a 3-6 membered carbocycle, wherein any C₁₋₆ alkyl is unsubstituted or is substituted with one or more R¹³;
R⁴, when present, is H;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹² is independently selected from C₁₋₆alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from a 3-6 membered carbocycle, a 3-6 membered heterocycle, C₁₋₆ alkyl, C₂₋₆ alkenyl, and H, wherein any C₁₋₆alkyl is optionally deuterated;
each R²⁰ is independently selected from —OH, —OC₁₋₆alkyl, —OC₁₋₆haloalkyl, —O, —CN, —NH₂, —NHC₁₋₆alkyl, —C(O)(C₁₋₆alkyl), a 3-6 membered carbocycle, phenyl, and halogen;
R²⁸ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen;
R^{a} and R^{b} are each independently selected from deuterium, halogen, C₁₋₆ alkyl, —OR², a 3-6 membered carbocycle, and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³:
each R^{d} is independently selected from H, deuterium, —OR¹², ═O, —C(O)(C₁₋₆alkylene)CN, —C(O)(C₁₋₆alkylene)OH, —C(O(C₁₋₆alkyl), —C(O)(3-6 membered carbocycle), —C(O)N(R¹⁴)₂, —S(O)₂(C₁₋₆alkyl), halogen, and C₁₋₆alkyl, wherein any C₁₋₆alkyl is unsubstituted or substituted with one or more R²⁰, and wherein any 3-6 membered carbocycle is unsubstituted or substituted with one or more R¹² or R²⁰
R^{e}, if present, is selected from —C(OC₁₋₆alkylene)CN, —C(O)(C₁₋₆alkylene)OH, —C(O)(C₁₋₆alkyl), —C(O)O(C₁₋₆alkyl), —C(O)OR¹⁴, —C(O)(3-6 membered carbocycle), —C(O)N(R)₂, —C(O)(3-8 membered heterocycle), —C(O)(5-6 membered heteraryl), —C(O)O(3-6 membered carbocycle), —C(O)O(3-6 membered heterocycle), —C(O)O(C₁₋₆alkylene)(3-6 membered heterocycle), —S(O)₂(C₁₋₆alkyl), a 5-6 membered heteroaryl, a 3-6 membered carbocycle, and C₁₋₆alkyl, wherein any C₁₋₆alkyl is optionally deuterated and is unsubstituted or substituted with one or more R¹⁰, and wherein any 3-6 membered carbocycle, 5-6 membered heteroaryl, or 3-6 or 3-8 membered heterocycle is unsubstituted or substituted with one or more R¹² or R²⁰;
each R^{f} is optionally absent and, if present, is selected from ═O, —NH₂, —NHC₁₋₆alkyl, and —N(C₁₋₆alkyl)₂;
X is selected from N and C—CN;
Y is selected from O and S;
R²³ is selected from —N(R¹⁰)₂, C₁₋₆alkyl, and C₁₋₆akyl-N(R¹⁴)₂, wherein any C₁₋₆alkyl is unsubstituted or substituted with one or more R¹³; and
R²⁴, R²⁵, and R²⁶ are independently selected from H, deuterium, halogen, and C₁₋₆alkyl, wherein any C₁₋₆alkyl is unsubstituted or substituted with one or more R¹³.

In some embodiments, the compound is a compound according to Formula IIA1, or a salt (e.g., pharmaceutically acceptable salt), ester, tautomer, prodrug, zwitterionic form, or stereoisomer(s) thereof. In some embodiments, the compound is a compound according to Formula IIB1, or a salt (e.g., pharmaceutically acceptable salt), ester, tautomer, prodrug, zwitterionic form, or stereoisomer(s) thereof. In some embodiments, the compound is a compound according to Formula IIC1, or a salt (e.g., pharmaceutically acceptable salt), ester, tautomer, prodrug, zwitterionic form, or stereoisomer(s) thereof. In some embodiments, the compound is a compound according to Formula IID1, or a salt (e.g., pharmaceutically acceptable salt), ester, tautomer, prodrug, zwitterionic form, or stereoisomer(s) thereof. In some embodiments, the compound is a compound according to Formula IIE1, or a salt (e.g., pharmaceutically acceptable salt), ester, tautomer, prodrug, zwitterionic form, or stereoisomer(s) thereof. In some embodiments, the compound is a compound according to Formula IIF1, or a salt (e.g., pharmaceutically acceptable salt), ester, tautomer, prodrug, zwitterionic form, or stereoisomer(s) thereof. In some embodiments, the compound is a compound according to Formula IIG1, or a salt (e.g., pharmaceutically acceptable salt), ester, tautomer, prodrug, zwitterionic form, or stereoisomer(s) thereof. In some embodiments, the compound is a compound according to Formula IIH1, or a salt (e.g., pharmaceutically acceptable salt), ester, tautomer, prodrug, zwitterionic form, or stereoisomer(s) thereof. In some embodiments, the compound is a compound according to Formula IIJ1, or a salt (e.g., pharmaceutically acceptable salt), ester, tautomer, prodrug, zwitterionic form, or stereoisomer(s) thereof. In some embodiments, the compound is a compound according to Formula IIK1, or a salt (e.g., pharmaceutically acceptable salt), ester, tautomer, prodrug, zwitterionic form, or stereoisomer(s) thereof. In some embodiments, the compound is a compound according to Formula IIL1, or a salt (e.g., pharmaceutically acceptable salt), ester, tautomer, prodrug, zwitterionic form, or stereoisomer(s) thereof. In some embodiments, the compound is a compound according to Formula IIM1, or a salt (e.g., pharmaceutically acceptable salt), ester, tautomer, prodrug, zwitterionic form, or stereoisomer(s) thereof. In some embodiments, the compound is a compound according to Formula IIN1, or a salt (e.g., pharmaceutically acceptable salt), ester, tautomer, prodrug, zwitterionic form, or stereoisomer(s) thereof. In some embodiments, the compound is a compound according to Formula IIP1, or a salt (e.g., pharmaceutically acceptable salt), ester, tautomer, prodrug, zwitterionic form, or stereoisomer(s) thereof. In some embodiments, the compound is a compound according to Formula IIQ1, or a salt (e.g., pharmaceutically acceptable salt), ester, tautomer, prodrug, zwitterionic form, or stereoisomer(s) thereof. In some embodiments, the compound is a compound according to Formula IIR1, or a salt (e.g., pharmaceutically acceptable salt), ester, tautomer, prodrug, zwitterionic form, or stereoisomer(s) thereof. In some embodiments, the compound is a compound according to Formula IIS1, or a salt (e.g., pharmaceutically acceptable salt), ester, tautomer, prodrug, zwitterionic form, or stereoisomer(s) thereof. In some embodiments, the compound is a compound according to Formula IIT1, or a salt (e.g., pharmaceutically acceptable salt), ester, tautomer, prodrug, zwitterionic form, or stereoisomer(s) thereof. In some embodiments, the compound is a compound according to Formula IIU1, or a salt (e.g., pharmaceutically acceptable salt), ester, tautomer, prodrug, zwitterionic form, or stereoisomer(s) thereof. In some embodiments, the compound is a compound according to Formula IIV1, or a salt (e.g., pharmaceutically acceptable salt), ester, tautomer, prodrug, zwitterionic form, or stereoisomer(s) thereof. In some embodiments, the compound is a compound according to Formula IIW1, or a salt (e.g., pharmaceutically acceptable salt), ester, tautomer, prodrug, zwitterionic form, or stereoisomer(s) thereof. In some embodiments, the compound is a compound according to Formula IIX1, or a salt (e.g., pharmaceutically acceptable salt), ester, tautomer, prodrug, zwitterionic form, or stereoisomer(s) thereof. In some embodiments, the compound is a compound according to Formula IIY1, or a salt (e.g., pharmaceutically acceptable salt), ester, tautomer, prodrug, zwitterionic form, or stereoisomer(s) thereof. In some embodiments, the compound is a compound according to Formula IIZ1, or a salt (e.g., pharmaceutically acceptable salt), ester, tautomer, prodrug, zwitterionic form, or stereoisomer(s) thereof. In some embodiments, the compound is a compound according to Formula IIA1, or a salt (e.g., pharmaceutically acceptable salt) thereof. In some embodiments, the compound is a compound according to Formula IIB1, or a salt (e.g., pharmaceutically acceptable salt) thereof. In some embodiments, the compound is a compound according to Formula IIC1, or a salt (e.g., pharmaceutically acceptable salt) thereof. In some embodiments, the compound is a compound according to Formula IID1, or a salt (e.g., pharmaceutically acceptable salt) thereof. In some embodiments, the compound is a compound according to Formula IIE1, or a salt (e.g., pharmaceutically acceptable salt) thereof. In some embodiments, the compound is a compound according to Formula IIF1, or a salt (e.g., pharmaceutically acceptable salt) thereof. In some embodiments, the compound is a compound according to Formula IIG1, or a salt (e.g., pharmaceutically acceptable salt) thereof. In some embodiments, the compound is a compound according to Formula IIH1, or a salt (e.g., pharmaceutically acceptable salt) thereof. In some embodiments, the compound is a compound according to Formula IIJ1, or a salt (e.g., pharmaceutically acceptable salt) thereof. In some embodiments, the compound is a compound according to Formula IIK1, or a salt (e.g., pharmaceutically acceptable salt) thereof. In some embodiments, the compound is a compound according to Formula IIL1, or a salt (e.g., pharmaceutically acceptable salt) thereof. In some embodiments, the compound is a compound according to Formula IIM1, or a salt (e.g., pharmaceutically acceptable salt) thereof. In some embodiments, the compound is a compound according to Formula IIN1, or a salt (e.g., pharmaceutically acceptable salt) thereof. In some embodiments, the compound is a compound according to Formula IIP1, or a salt (e.g., pharmaceutically acceptable salt) thereof. In some embodiments, the compound is a compound according to Formula IIQ1, or a salt (e.g., pharmaceutically acceptable salt) thereof. In some embodiments, the compound is a compound according to Formula IIR1, or a salt (e.g., pharmaceutically acceptable salt) thereof. In some embodiments, the compound is a compound according to Formula IIS1, or a salt (e.g., pharmaceutically acceptable salt) thereof. In some embodiments, the compound is a compound according to Formula IIT1, or a salt (e.g., pharmaceutically acceptable salt) thereof. In some embodiments, the compound is a compound according to Formula IIU1, or a salt (e.g., pharmaceutically acceptable salt) thereof. In some embodiments, the compound is a compound according to Formula IIV1, or a salt (e.g., pharmaceutically acceptable salt) thereof. In some embodiments, the compound is a compound according to Formula IIW1, or a salt (e.g., pharmaceutically acceptable salt) thereof. In some embodiments, the compound is a compound according to Formula IIX1, or a salt (e.g., pharmaceutically acceptable salt) thereof. In some embodiments, the compound is a compound according to Formula IIY1, or a salt (e.g., pharmaceutically acceptable salt) thereof. In some embodiments, the compound is a compound according to Formula IIZ1, or a salt (e.g., pharmaceutically acceptable salt) thereof.

In some embodiments, the compound is a compound according to Formula IIAA1:

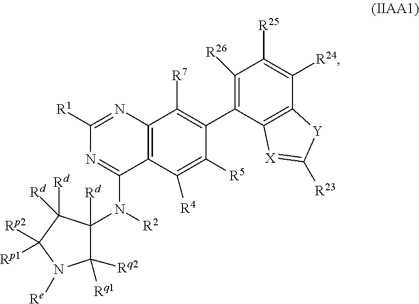

or a salt (e.g., pharmaceutically acceptable salt), ester, tautomer, prodrug, zwitterionic form, or stereoisomer(s) thereof, wherein:
R¹ is selected from —OR⁸ and

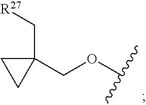

R² is selected from H, C₁₋₆alkyl, and a 3-6 membered carbocycle, wherein any C₁₋₆ alkyl is unsubstituted or is substituted with one or more R¹³;
R⁴ is H;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety ofany alkylheterocycle is selected from C₁₋₆alkyl;
each R¹² is independently selected from C₁₋₄ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R⁴ is independently selected from a 3-6 membered carbocycle, a 3-6 membered heterocycle, C₁₋₆ alkyl, C₂₋₆ alkenyl, and H, wherein any C₁₋₆alkyl is optionally deuterated;
each R²⁸ is independently selected from —OH, —OC₁₋₆alkyl, —OC₁₋₆haloalkyl, ═O, —CN, —NH₂, —NHC₁₋₆alkyl, —C(O)(C₁₋₆alkyl), a 3-6 membered carbocycle, phenyl, and halogen;
R⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen;
R^{a} and R^{b} are each independently selected from deuterium, halogen, C₁₋₆ alkyl, —OR¹², a 3-6 membered carbocycle, and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R²⁸;
each R^{d} is independently selected from H, deuterium, —OR¹², ═O, —C(O)(C₁₋₆alkylene)CN, —C(O(C₁₋₆alkylene)OH, —C(O)(C₁₋₆alkyl), —C(O)(3-6 membered carbocycle), —C(O)N(R¹⁴)₂, —S(O)₂(C₁₋₆alkyl), a 3-6 membered carbocycle, a 3-6 membered heterocycle, halogen, and C₁₋₆alkyl, wherein any C₁₋₆alkyl is unsubstituted or substituted with one or more R²⁰, and wherein any 3-6 membered carbocycle or 3-6 membered heterocycle is unsubstituted or substituted with one or more R¹² or R²⁰;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³;
R²⁴, R²⁵, and R²⁶ are independently selected from H, deuterium, halogen, and C₁₋₆-alkyl, wherein any C₁₋₆alkyl is unsubstituted or substituted with one or more R¹³; and
(i) R^{q1}, R^{q2}, and R^{p2} are each independently selected from R^{d}, and R^{e} and R^{p1}, together with the atoms to which they are attached, form a 5-6 membered heterocycle that is unsubstituted or is substituted with one or more R^{d}; or
(ii) R^{p1}, R^{p2}, and R^{q2} are each independently selected from R^{d}, and R^{e} and R^{q1}, together with the atoms to which they are attached, form a 5-6 membered heterocycle that is unsubstituted or is substituted with one or more R^{d}.

In some embodiments, the compound is a compound according to Formula IIAA1, or a salt (e.g., pharmaceutically acceptable salt) thereof.

In some embodiments, for a compound according to Formula IIAA or IIAA1, R^{q1}, R^{q2}, and R^{p2} are each independently selected from R^{d}, and R^{e} and R^{p1}, together with the atoms to which they are attached, form a 5-6 membered heterocycle that is unsubstituted or is substituted with one or more R^{d}. In some embodiments, R^{e} and R^{P}, together with the atoms to which they are attached, form a 5-membered heterocycle that is unsubstituted or is substituted with one or more R^{d}. In some embodiments, R^{e} and R^{p1}, together with the atoms to which they are attached, form a 6-membered heterocycle that is unsubstituted or is substituted with one or more R^{d}. In some embodiments, R^{e} and R^{p1}, together with the atoms to which they are attached, form a 5-6 membered heterocycle that is unsubstituted. In some embodiments, RV and R^{p1}, together with the atoms to which they are attached, form a 5-6 membered heterocycle that is substituted with one or more R^{d}, provided that at least one R^{d} is not H. In some embodiments, R^{e} and R^{p1}, together with the atoms to which they are attached, form a piperidine, oxazinane, oxazolidine, imidazolidine, or pyrrolidine that is unsubstituted or is substituted with one or more R^{d}. In some embodiments, R^{e} and R^{p1}, together with the atoms to which they are attached, form a morpholine, piperidine, oxazinane, oxazolidine, imidazolidine, or pyrrolidine that is unsubstituted or is substituted with one or more R^{d}. In some embodiments, R^{p1} is H.

In some embodiments, for a compound according to Formula IIAA or IIAA1, R^{p1}, R^{p2}, and R^{q2} are each independently selected from R^{d}, and R^{e} and R^{q1}, together with the atoms to which they are attached, form a 5-6 membered heterocycle that is unsubstituted or is substituted with one or more R^{d}. In some embodiments, R^{e} and R^{q1}, together with the atoms to which they are attached, form a 5-membered heterocycle that is unsubstituted or is substituted with one or more R^{d}. In some embodiments, R^{e} and R^{q1}, together with the atoms to which they are attached, form a 6-membered heterocycle that is unsubstituted or is substituted with one or more R^{d}. In some embodiments, R^{e} and R^{q1}, together with the atoms to which they are attached, form a 5-6 membered heterocycle that is unsubstituted. In some embodiments, R^{e} and R^{q1}, together with the atoms to which they are attached, form a 5-6 membered heterocycle that is substituted with one or more R^{d}, provided that at least one R^{d} is not H. In some embodiments, R^{e} and R^{q1}, together with the atoms to which they are attached, form a piperidine, oxazinane, oxazolidine, imidazolidine, or pyrrolidine that is unsubstituted or is substituted with one or more R^{d}. In some embodiments, R^{c} and R^{p1}, together with the atoms to which they are attached, form a morpholine, piperidine, oxazinane, oxazolidine, imidazolidine, or pyrrolidine that is unsubstituted or is substituted with one or more R^{d}. In some embodiments, R^{q2} is H.

In some embodiments, for a compound according to any one of Formulas IIA1, IIB1, IIC1, IID1, IIE1, IIF1, IIG1, IIH1, IIJ1, IIK1, IIL1, IIM1, IIN1, IIP1, IIQ1, IIR1, IIS1, IIT1, IIU1, IIV1, IIW1, IIX1, IIY1, IIZ1, and IIAA1, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R¹²)₂. In some embodiments, X is C—CN, Y is S, and R²³ is —NH₂. In some embodiments, X is C—CN, Y is S, R²³ is —N(R¹²)₂, and R²³ is a halogen (e.g., F). In some embodiments, X is C—CN, Y is S, R²³ is —N(R¹²)₂, and one or more of R²⁴, R²⁵, and R²⁶ is a halogen (e.g., F). In some embodiments, R²⁶ is deuterium.

In some embodiments, for a compound according to any one of Formulas IIC, IIC1, IIE, IIE1, IIL, IIL1, IIF1, IIG1, IIH1, IIJ1, IIK1, IIL1, IIM1, IIN1, IIP1, IIQ1, IIR1, IIS1, IIT1, IIU1, IIV1, IIW, IIW1, IIX, IIX1, IIY, IIY1 and IIZ1, each R²³ is ═O. In some embodiments, each R^{f} optionally absent and, if present, is ═O. In some embodiments, each R^{f} is absent.

In some embodiments, fora compound of any one of Formulas IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, and IIAA1, each R^{d} is H. In some embodiments, at least one R^{d} is selected from —OR¹² and a C₁₋₆alkyl substituted with —OH. In some embodiments, each R^{d} is independently selected from H, —OR¹², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆ alkyl that is unsubstituted or substituted with one or more R²⁰. It will be appreciated that when R^{d} is ═O, the other R^{d} on the same carbon atom is absent, such that the carbon has proper valency.

In some embodiments, for a compound according to anyone of Formulas IIA, IIA1, IID, IID1, IIG, IIG1, IIH, IIH1, IIN, IIN1, IIR, IIR1, IIU, IIU1, IIV, and IIV1, R^{e} is selected from —C(O)(C₁₋₆alkylene)CN, —C(O)(C₁₋₆alkylene)OH, —C(O)(C₁₋₆alkyl), —C(O)(3-6 membered carbocycle), —C(O)N(R¹⁴)₂, —C(O)OR¹⁴, —S(O)₂(C₁₋₆alkyl), and C₁₋₆alkyl, wherein any C₁₋₆alkyl is unsubstituted or substituted with one or more R¹³; and wherein any 3-6 membered carbocycle is unsubstituted or substituted with one or more R¹² or R²⁰. In some embodiments, R^{e} is selected from —C(O)(C₁₋₆alkylene)CN, —C(O)(C₁₋₆alkylene)OH, —C(O)(C₁₋₆alkyl), —C(O)O(C₁₋₆alkyl), —C(O)OR¹⁴, —C(O)(3-6 membered carbocycle), —C(O)(3-7 membered heterocycle), —C(O)(5-6 membered heteroaryl), —C(O)O(3-6 membered heterocycle), and —C(O)O(C₁₋₆alkylene)(3-6 membered heterocycle), wherein any C₁₋₆alkyl is unsubstituted or substituted with one or more R²⁰, and wherein any 3-6 membered carbocycle, 5-6 membered heteroaryl, or 3-6 membered heterocycle is unsubstituted or substituted with one or more R¹² or R²⁰. In some embodiments, R^{e} is selected from —C(O)(3-6 membered carbocycle), —C(O)(3-7 membered heterocycle), —C(O)(5-6 membered heteroaryl), —C(O)O(3-6 membered heterocycle), and —C(O)O(C₁₋₆alkylene)(3-6 membered heterocycle), wherein any C₁₋₆alkyl is unsubstituted or substituted with one or more R¹⁰, and wherein any 3-6 membered carbocycle, 5-6 membered heteroaryl, or 3-6 membered heterocycle is unsubstituted or substituted with one or more R¹² or R²⁰. In some embodiments, R^{e} is selected from —C(O)(C₁₋₆alkylene)CN, —C(O)(C₁₋₆alkylene)OH, —C(O(C₁₋₆alkyl), —C(O)N(R¹⁴)₂, —C(O)OR¹⁴, and C₁₋₆alkyl, wherein any C₁₋₆alkyl is unsubstituted or substituted with one or more R²³. In some embodiments, R^{e} is selected from —C(O)(C₁₋₆alkyl), —C(O)(3-6 membered carbocycle), and —C(O)O(C₁₋₆alkyl), wherein any C₁₋₆ alkyl is unsubstituted or substituted with one or more R¹⁰, and any 3-6 membered carbocycle is unsubstituted or substituted with one or more R¹² or R²⁰. In some embodiments, R¹ is C₁₋₆alkyl that is unsubstituted or substituted with one or more R²⁰. In some embodiments, R^{e} is C₁₋₆alkyl that is substituted with one or more R²⁰. In some embodiments, R²⁰ is C₁₋₆alkyl that is substituted with one or more R²⁰, wherein each R²⁰ is independently selected from —OH, —OC₁₋₆alkyl, ═O, and —CN. In some embodiments, R¹ is selected from —C(O(C₁₋₆alkyl), wherein any C₁₋₆alkyl is unsubstituted or substituted with one or more R²⁰. In some embodiments, R^{e} is selected from —C(O)(C₁₋₆alkyl), wherein any C₁₋₆alkyl is unsubstituted. In some embodiments, R^{e} is selected from —C(O)(C₁₋₆alkyl), wherein any C₁₋₆alkyl is substituted with one or more R²⁰, wherein the one or more R²⁰ are independently selected from —OC₁₋₆alkyl, halogen, —OH, and —CN. In some embodiments, R^{e} is selected from —C(O)(C₁₋₆alkylene)CN, wherein any C₁₋₆alkyl is unsubstituted or substituted with one or more R¹⁰. In some embodiments, R^{e} is selected from —C(O)(C₁₋₆alkylene)OH, wherein any C₁₋₆alkyl is unsubstituted or substituted with one or more R²⁰. In some embodiments, R^{e} is selected from —C(O)N(R¹²)₂. In some embodiments, R^{e} is selected from —C(O)N(R¹⁴)₂, wherein each R¹⁴ is independently selected from a 3-6 membered carbocycle, a 3-6 membered heterocycle, C₁₋₆ alkyl, and H. In some embodiments, R^{e} is selected from —C(O)N(R¹⁴)₂, wherein each R¹⁴ is independently selected from C₁₋₆ alkyl and H. In some embodiments, when R^{e} is —C(O)N(R¹⁴)₂, then at least one R¹⁴ is not H. In some embodiments, when R^{e} is —C(O)N(R¹²)₂, then each R¹⁴ is C₁₋₆ alkyl (e.g., methyl or ethyl). In some embodiments, R^{e} is selected from —C(O)OR¹⁴. In some embodiments, R^{e} is selected from —C(O)OR¹⁴, wherein R¹⁴ is selected from a 3-6 membered carbocycle, a 3-6 membered heterocycle, C₁₋₆ alkyl, and C₂₋₆ alkenyl, wherein any C₁₋₆ alkyl is optionally deuterated. In some embodiments, R^{e} is selected from —C(O)OR¹⁴, wherein R¹⁴ is selected from a 3-6 membered carbocycle and a 3-6 membered heterocycle. In some embodiments, R^{e} is —S(O)₂(C₁₋₆alkyl), wherein the C₁₋₆alkyl is unsubstituted or substituted with one or more R²⁰. In some embodiments, R^{e} is selected from —C(O)(3-6 membered carbocycle), wherein the 3-6 membered carbocycle is unsubstituted or substituted with one or more R¹² or R²⁰. In some embodiments, R^{e} is —C(O)(3-6 membered carbocycle) that is unsubstituted. In some embodiments, R^{e} is selected from —C(O)(3-6 membered carbocycle), wherein the 3-6 membered carbocycle is substituted with one or more R″ or R″. In some embodiments, R^{e} is selected from —C(O)(3-6 membered carbocycle), wherein the 3-6 membered carbocycle is cyclopropane or cyclobutane, and wherein the 3-6 membered carbocycle is unsubstituted or substituted with one or more R¹² or R²⁰. In some embodiments, R^{e} is —C(O)(cyclopropane). In some embodiments, R is selected from —C(O(cyclopropane), wherein the cyclopropane is substituted with one or more R¹² or R²⁰ (e.g., one or more fluoro). In some embodiments, R²⁰ is selected from —C(O)(3-8 membered heterocycle), wherein the 3-8 membered heterocycle is unsubstituted or substituted with one or more R¹² or R²⁰. In some embodiments, R^{e} is selected from —C(O)(3-8 membered heterocycle), wherein the 3-8 membered heterocycle is unsubstituted. In some embodiments, R^{e} is selected from —C(O)(3-8 membered heterocycle), wherein the 3-8 membered heterocycle is substituted with one or more R¹² or R²⁰. In some embodiments, R^{e} is selected from —C(O)(3-7 membered heterocycle), wherein the 3-7 membered heterocycle is unsubstituted or substituted with one or more R¹² or R²⁰. In some embodiments, R^{e} is selected from —C(O)(3-7 membered heterocycle), wherein the 3-7 membered heterocycle is unsubstituted. In some embodiments, R^{e} is selected from —C(O)(3-7 membered heterocycle), wherein the 3-7 membered heterocycle is substituted with one or more R¹² or R²⁰. In some embodiments, R^{e} is selected from —C(O)(3-7 membered heterocycle), wherein the 3-7 membered heterocycle is oxetane, azetidine, pyrrolidine, azabicyclo[3.1.0]hexane, morpholine, a bridged morpholine, tetrahydropyran, piperidine, piperazine, or 2-oxa-6-azaspiro[3.3]heptane, and wherein the 3-7 membered heterocycle is unsubstituted or substituted with one or more R¹² or R²⁰. In some embodiments, R^{e} is selected from —C(O)(3-7 membered heterocycle), wherein the 3-7 membered heterocycle is oxctane, azetidine, pyrrolidine, morpholine, tetrahydropyran, piperidine, or piperazine, and wherein the 3-7 membered heterocycle is unsubstituted or substituted with one or more R¹² or R²⁰. In some embodiments, R^{e} is selected from —C(O)(3-7 membered heterocycle), wherein the 3-7 membered heterocycle is a morpholine or bridged morpholine, and wherein the morpholine or bridged morpholine is unsubstituted or substituted with one or more R¹² or R²⁰. In some embodiments, R^{e} is selected from —C(O)(3-7 membered heterocycle), wherein the 3-7 membered heterocycle is a bridged morpholine selected from

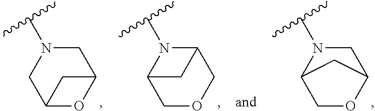 [SMILES: *N1CC2CC(C1)O2 ; *N1C2COCC1C2 ; *N1CC2CC1CO2]

and wherein the bridged morpholine is unsubstituted or substituted with one or more R¹² or R²⁰. In some embodiments, R^{e} is selected from —C(O)(3-7 membered heterocycle), wherein the 3-7 membered heterocycle is substituted with one or more R¹² or R²⁰, wherein each R² is independently selected from C₁₋₆alkyl and each R^{a} is independently selected from —OC₁₋₆alkyl, —C(O(C₁₋₆alkyl), and halogen. In some embodiments, R^{e} is selected from —C(O)(5-6 membered heteroaryl), wherein the 5-6 membered heteroaryl is unsubstituted or substituted with one or more R¹² or R²⁰. In some embodiments, R^{e} is selected from —C(O)(5-6 membered heteroaryl), wherein the 5-6 membered heteroaryl is unsubstituted. In some embodiments, R^{e} is selected from —C(O)(5-6 membered heteroaryl), wherein the 5-6 membered heteroaryl is substituted with one or more R¹² or R²⁰. In some embodiments, R¹ is selected from —C(O)(5-6 membered heteroaryl), wherein the 5-6 membered heteroaryl is thiophene, pyrazole, oxazole, isoxazole, thiazole, isothiazole, triazole, oxadiazole, thiadiazole, pyridine, or pyrimidine, any of which is unsubstituted or substituted with one or more R¹² or R²⁰. In some embodiments, R^{e} is selected from —C(O)(5-6 membered heteroaryl), wherein the 5-6 membered heteroaryl is oxazole or isoxazole, and wherein the oxazole or isoxazole is unsubstituted or substituted with one or more R¹² or R²⁰. In some embodiments, R^{e} is selected from —C(O)O(3-6 membered carbocycle), wherein the 3-6 membered carbocycle is unsubstituted or substituted with one or more R¹² or R²⁰. In some embodiments, R^{e} is selected from —C(O)O(3-6 membered carbocycle), wherein the 3-6 membered carbocycle is unsubstituted. In some embodiments, R^{e} is selected from —C(O)O(3-6 membered carbocycle), wherein the 3-6 membered carbocycle is substituted with one or more R¹² or R²⁰. In some embodiments, R^{e} is selected from —C(O)O(3-6 membered carbocycle), wherein the 3-6 membered carbocycle is cyclopropane or cyclobutane, and wherein the 3-6 membered carbocycle is unsubstituted or substituted with one or more R¹² or R²⁰. In some embodiments, R^{e} is selected from —C(O)O(3-6 membered heterocycle), wherein the 3-6 membered heterocycle is unsubstituted or substituted with one or more R¹² or R²⁰. In some embodiments, R^{e} is selected from —C(O)O(3-6 membered heterocycle), wherein the 3-6 membered heterocycle is unsubstituted. In some embodiments, R^{e} is selected from —C(O)O(3-6 membered heterocycle), wherein the 3-6 membered heterocycle is substituted with one or more R¹² or R²⁰. In some embodiments, R^{e} is selected from —C(O)O(3-6 membered heterocycle), wherein the 3-6 membered heterocycle is oxctane, tetrahydrofuran, tetrahydropyran, or pyrrolidine, and wherein the 3-6 membered heterocycle is unsubstituted or substituted with one or more R¹² or R²⁰. In some embodiments, R^{e} is selected from —C(O)O(C₁₋₆akylene)(3-6 membered heterocycle), wherein the 3-6 membered heterocycle is unsubstituted or substituted with one or more R¹² or R²⁰. In some embodiments, R^{e} is selected from —C(O)O(C₁₋₆alkylene)(3-6 membered heterocycle), wherein the 3-6 membered heterocycle is unsubstituted. In some embodiments, R^{e} is selected from —C(O)O(C₁₋₆alkylene)(3-6 membered heterocycle), wherein the 3-6 membered heterocycle is substituted with one or more R¹² or R²⁰. In some embodiments, R^{e} is a 3-6 membered carbocycle, wherein the 3-6 membered carbocycle is unsubstituted or substituted with one or more R¹² or R²⁰. In some embodiments, R¹ is a 3-6 membered carbocycle, wherein the 3-6 membered carbocycle is unsubstituted. In some embodiments, R¹ is a 3-6 membered carbocycle, wherein the 3-6 membered carbocycle is substituted with one or more R¹² or R²⁰. In some embodiments, R^{e} is a 5-6 membered heteroaryl, wherein the 5-6 membered heteroaryl is unsubstituted or substituted with one or more R¹² or R²⁰. In some embodiments, R is a 5-6 membered heteroaryl, wherein the 5-6 membered heteroaryl is unsubstituted. In some embodiments, R^{e} is a 5-6 membered heteroaryl, wherein the 3-6 membered carbocycle is substituted with one or more R¹² or R²⁰.

In some embodiments, for a compound of any one of Formulas IID, IID1, IIR, and IIR1,

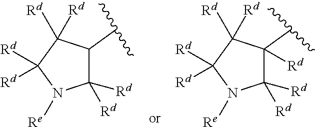

is a structure selected from:

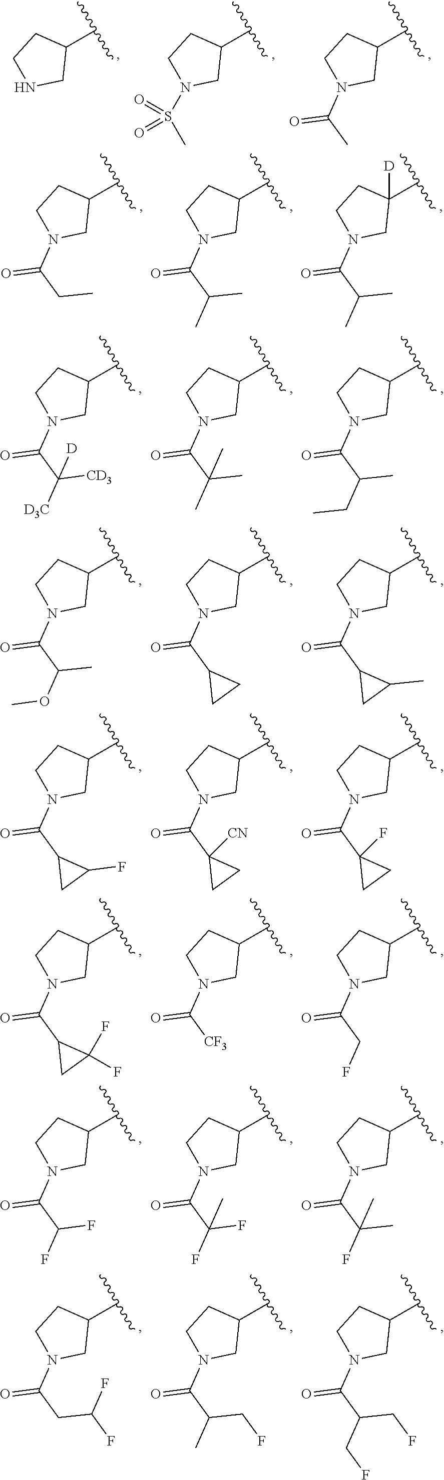

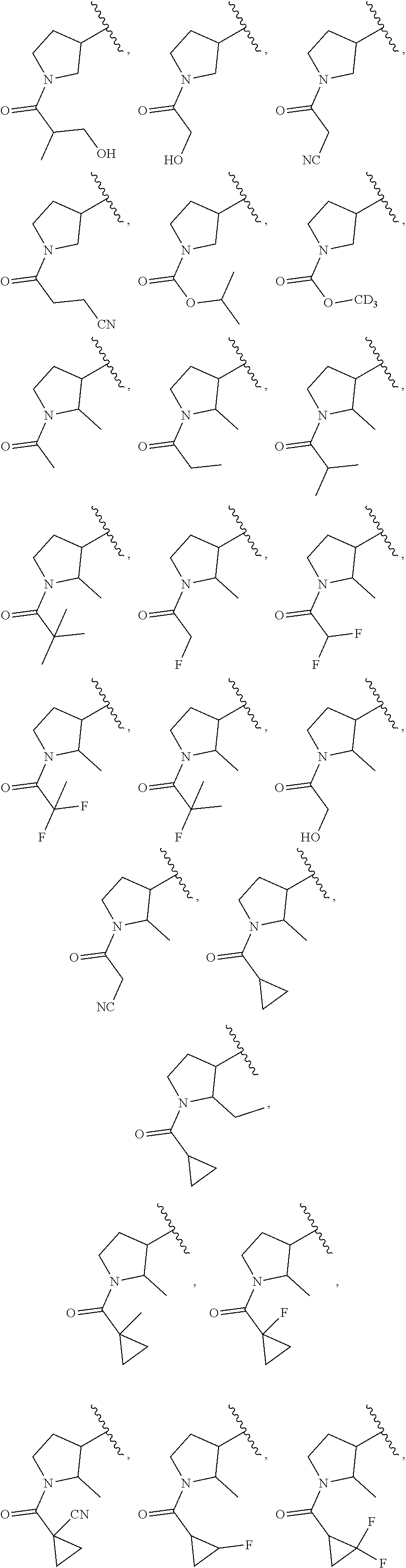

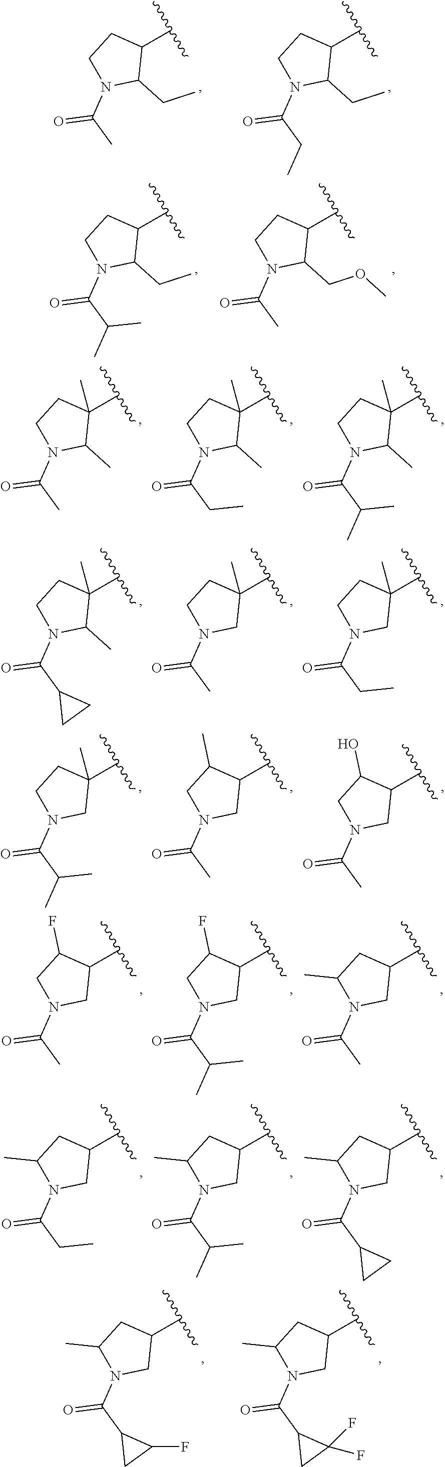

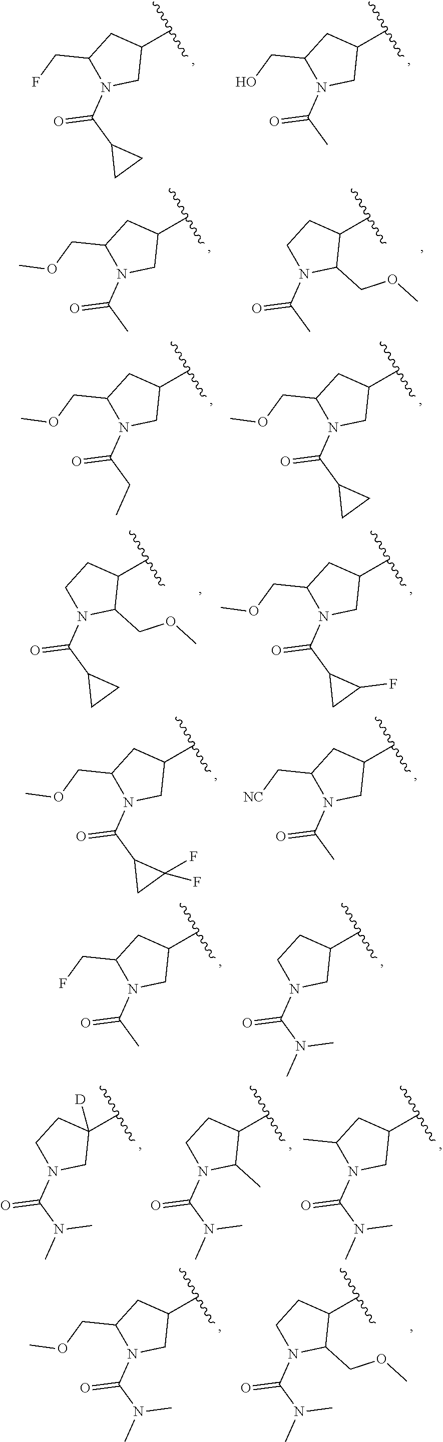

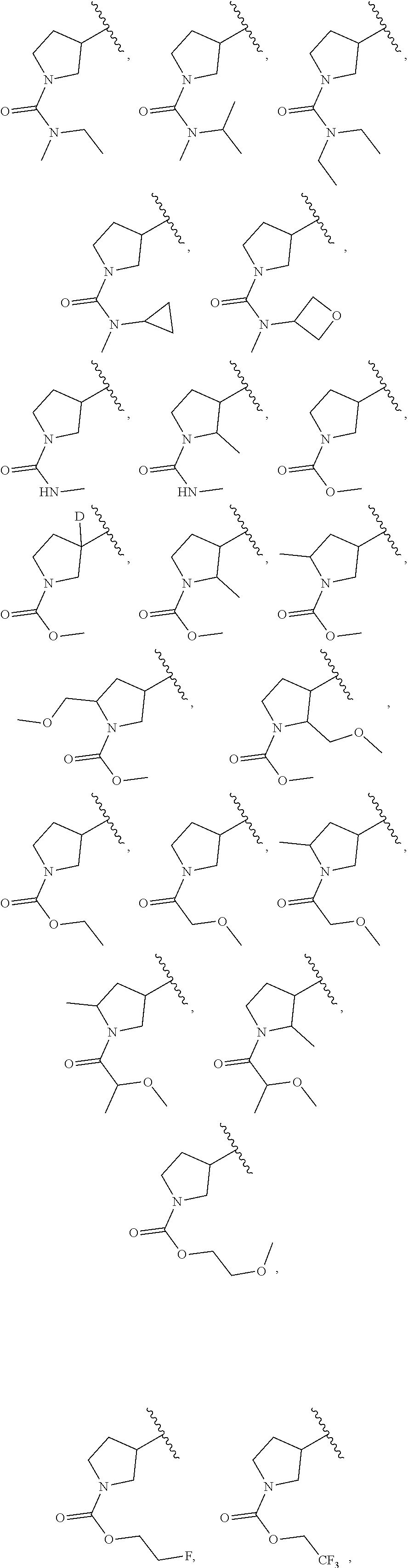

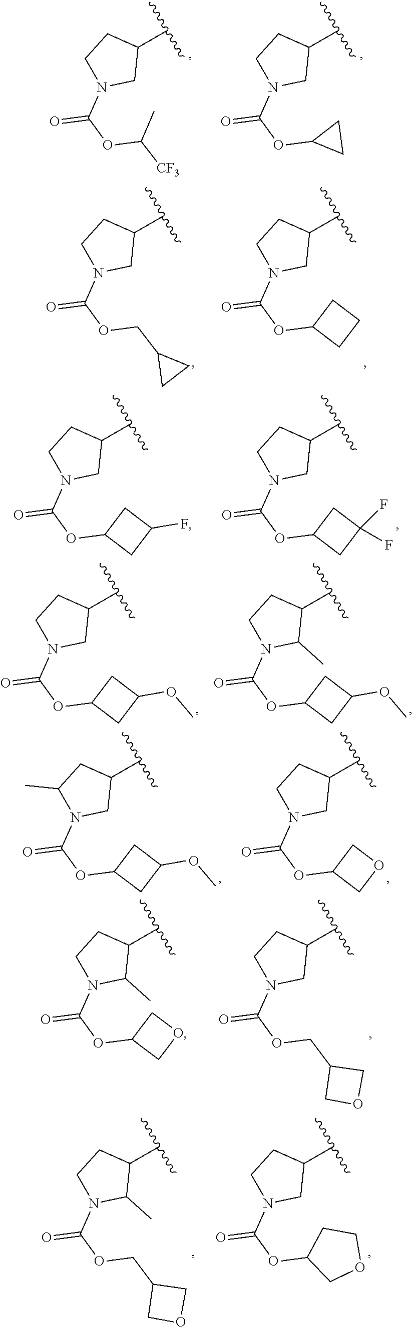

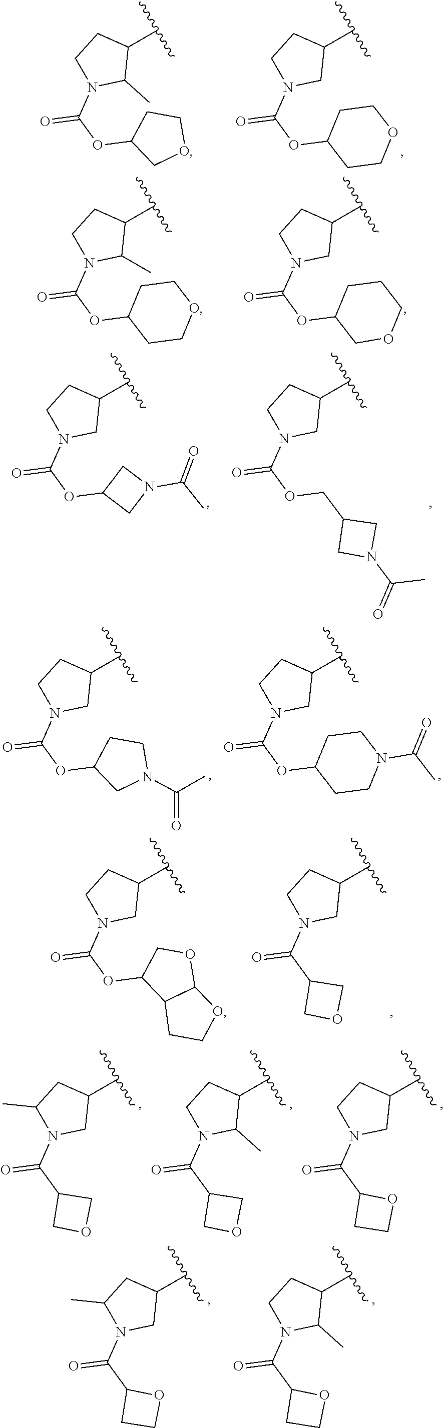

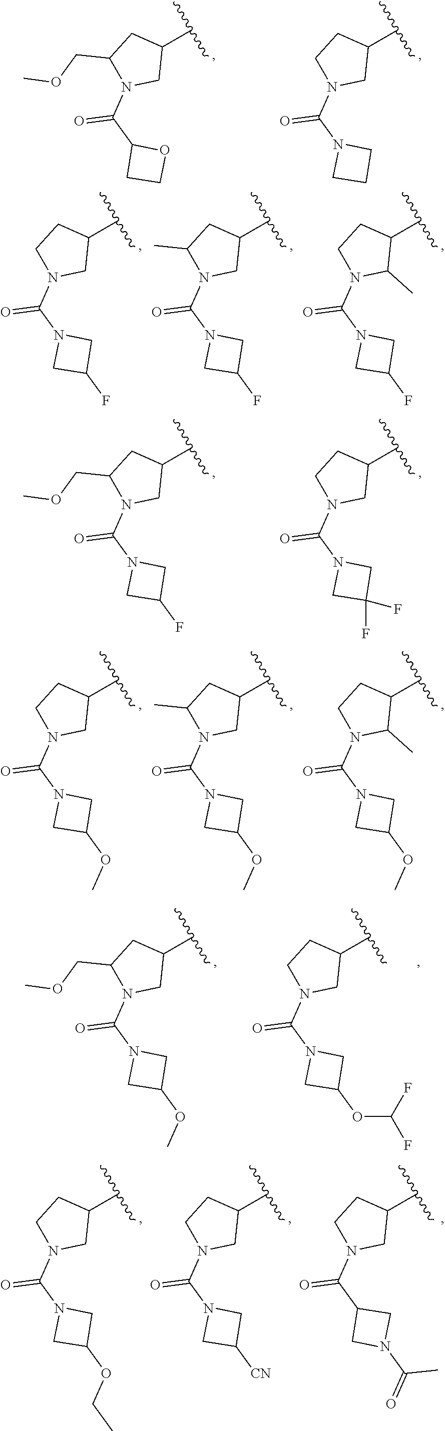

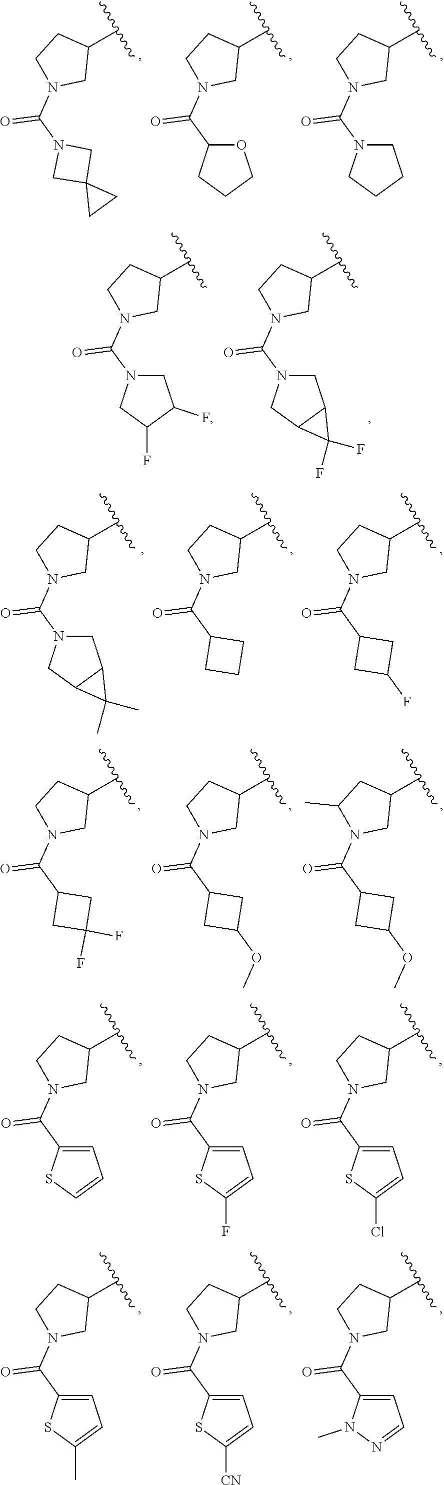

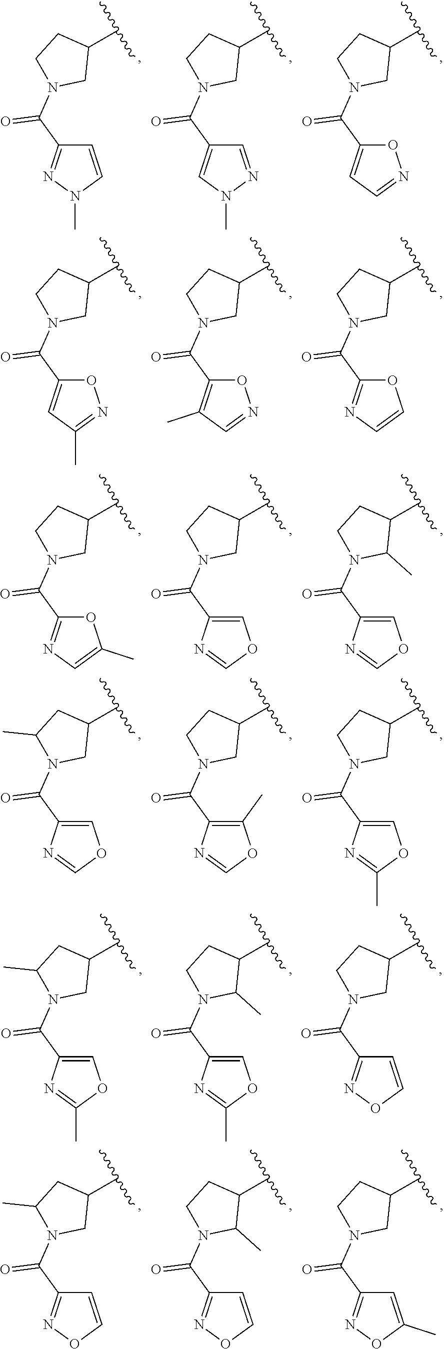

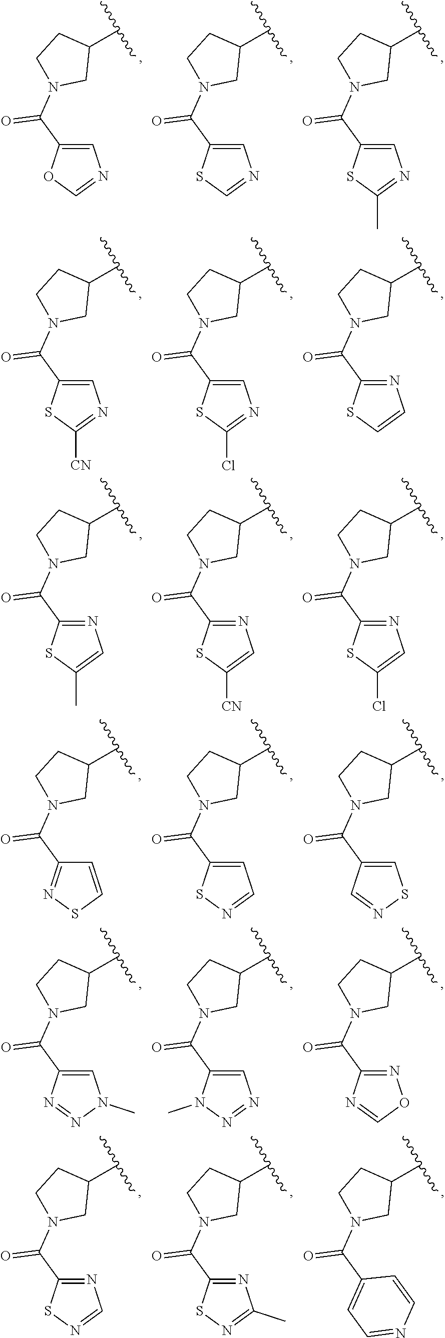

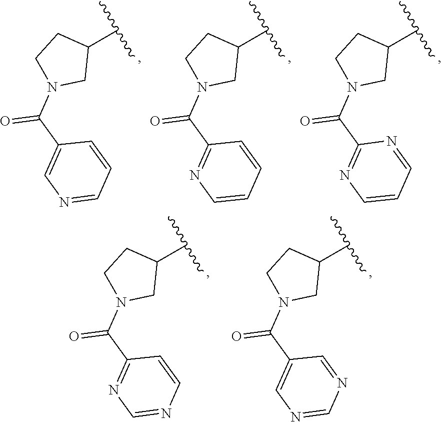

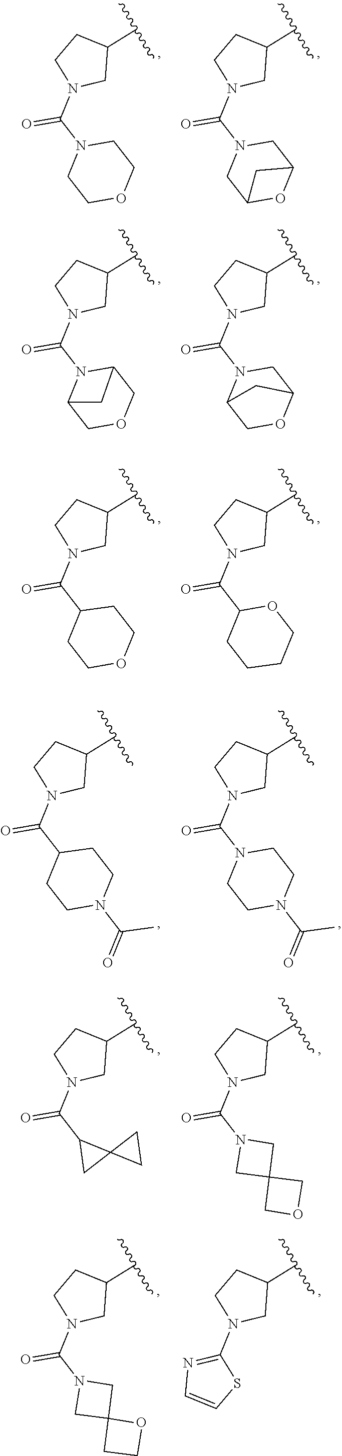

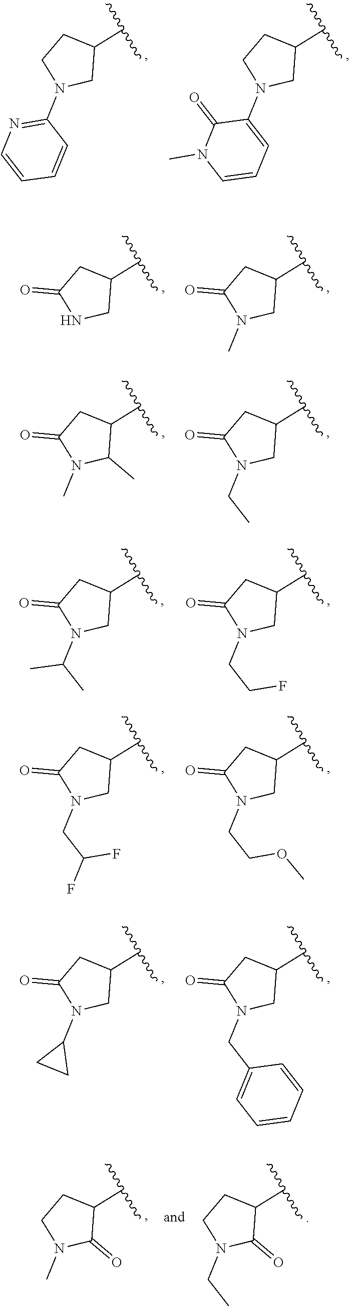

In some embodiments, for a compound of any one of Formulas II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, and IIAA1, R² is H. In some embodiments, R² is a C₁₋₆alkyl that is unsubstituted or is substituted with one or more R¹³. In some embodiments, R² is a C₁₋₆alkyl that is unsubstituted. In some embodiments, R² is a C₁₋₆alkyl that is substituted with one or more R¹⁰. In some embodiments, R² is a C₁₋₆alkyl that is unsubstituted or is substituted with one or more R¹³. In some embodiments, R² is a C₁₋₂alkyl that is unsubstituted. In some embodiments, R² is a C₁₋₂alkyl that is substituted with one or more R¹³. In some embodiments, R² is methyl. In some embodiments, R² is ethyl.

In some embodiments, for a compound of any one of Formulas IT, II′, IIA, IIA1, IIB, IIB1, IIC, IIC, IID, IID1, IIE, IIE1, IIF, IIF1, IIG, IIG1, IIH, IIH1, IIJ1, IIJ, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, and IIAA1, R⁸ is selected from —OR⁸, wherein R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl. In some embodiments, R⁸ is a heterocycle or an alkylheterocycle, wherein any heterocycle contains 4-8 members and is substituted with one or more R^{a} or R^{b}. In some embodiments, R⁸ is a heterocycle that is unsubstituted or substituted with one or more R^{a} or R^{b}. In some embodiments, R⁸ is an alkylheterocycle that is unsubstituted or substituted with one or more R^{a} or R^{b}. In some embodiments, R⁸ is —CH₂(heterocycle), where the heterocycle is unsubstituted or substituted with one or more R^{a} or R^{b}. In some embodiments, a heterocycle or a heterocycle of an alkylheterocycle is a 4-6 membered monocyclic heterocycle having 1-2 heteroatoms independently selected from N, O, and S. In some embodiments, a heterocycle or a heterocycle of an alkylheterocycle is an 8-membered bicyclic heterocycle having 1-2 heteroatoms independently selected from N, O, and S. In some embodiments, a heterocycle or a heterocycle of an alkylheterocycle is substituted with one or more R^{a} or R^{b}, wherein the one or more R^{a} or R^{b} is a halogen (e.g., F). In some embodiments, a heterocycle or a heterocycle of an alkylheterocycle is substituted with one or more R^{a} or R^{b}, wherein the one or more R^{a} or R^{b} is a C₁₋₆alkyl (e.g., methyl). In some embodiments, a heterocycle or a heterocycle of an alkylheterocycle is substituted with one or more R^{a} or R^{b}, wherein the one or more R^{a} or R^{b} is a —OR¹² (e.g., —OCH₃). In some embodiments, a heterocycle or a heterocycle of an alkylheterocycle is substituted with one or more R^{a} or R^{b}, wherein the one or more R^{a} or R^{b} is a 3-6 membered carbocycle (e.g., a cyclopropane). In some embodiments, a heterocycle or a heterocycle of an alkylheterocycle is substituted with one or more R^{a} or R^{b}, wherein the one or more R^{a} or R^{b} is deuterium.

In some embodiments, for a compound according to any one of Formulas II, I′, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, and IIAA1, R¹ is selected from:

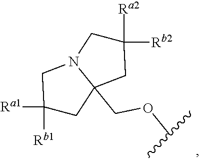

wherein R^{a1}, R^{a2}, R^{b1}, and R^{b2} are each independently selected from deuterium, halogen, C₁₋₆alkyl, —OR¹², and H, wherein R^{a1} and R^{b1} and/or R^{a2} and R^{b2} can optionally join together to form a 3-6 membered carbocycle, and wherein any C₁₋₆alkyl or 3-6 membered carbocycle is unsubstituted or is substituted with one or more R¹³. In some embodiments, R^{a1} and/or R^{a2} is a halogen. In some embodiments, R^{a1} and/or R^{a2} is F. In some embodiments, R^{a1} and/or R^{a2} is C₁₋₆alkyl that is unsubstituted or is substituted with one or more R¹³. In some embodiments, R^{a1} and/or R^{a2} is methyl. In some embodiments, R^{a1} and/or R¹² is —OC₁₋₆ alkyl. In some embodiments, R^{a1} and/or R^{a2} is H. In some embodiments, R^{a1} and/or R^{a2} is H. In some embodiments, R^{b1} and/or R^{b2} is a halogen. In some embodiments, R^{b1} and/or R²⁰ is F. In some embodiments, R^{b1} and/or R^{b2} is C₁₋₆alkyl that is unsubstituted or is substituted with one or more R¹³. In some embodiments, R^{b1} and/or R^{b2} is methyl. In some embodiments, each of R^{b1} and R^{b2} is F. In some embodiments, R^{b1} and/or R^{b2} is D. In some embodiments, R^{a2} and R^{b2} join together to form a 3-6 membered carbocycle (e.g., cyclopropane), which carbocycle is optionally substituted with one or more R¹³. In some embodiments, R^{a1} and R^{b1} join together to form a 3-6 membered carbocycle (e.g., cyclopropane). In some embodiments, R¹ is selected from:

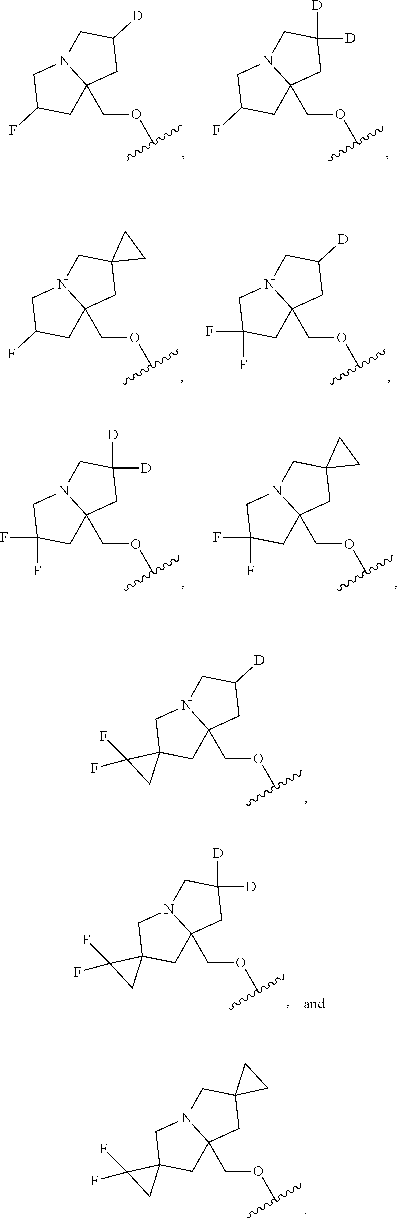

In some embodiments, for a compound of any one of Formulas II, I′, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, and IIAA1, R¹ is selected from:

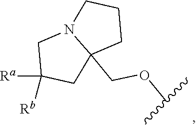

wherein R^{a} and R^{b} are each independently selected from halogen, C₁₋₆alkyl, —OR¹², and H, wherein any C₁₋₆ alkyl is unsubstituted or is substituted with one or more R¹³. In some embodiments, R²⁰ is a halogen. In some embodiments, R^{a} is F. In some embodiments, R^{a} is C₁₋₆alkyl that is unsubstituted or is substituted with one or more R¹³. In some embodiments, R^{a} is methyl. In some embodiments, R^{a} is —OC₁₋₆alkyl. In some embodiments, R^{a} is H. In some embodiments, R^{b} is H. In some embodiments, R^{b} is a halogen. In some embodiments, R^{b} is F. In some embodiments, R^{b} is C₁₋₆alkyl that is unsubstituted or is substituted with one or more R¹³. In some embodiments, R^{b} is methyl. In some embodiments, each of R^{a} and R^{b} is F. In some embodiments, each of R^{a} and R^{b} is methyl. In some embodiments, R¹ is selected from:

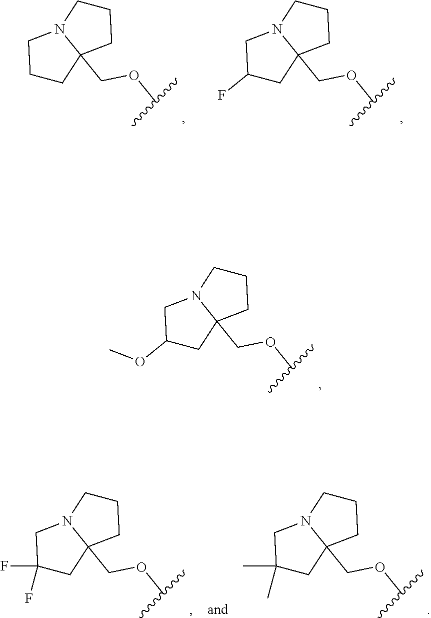 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

In some embodiments, for a compound of any one of Formulas II, II′, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IS, IIS1, IIT, IIT1, IIU, IIU1, IIV, IIV1, IIW, IIW1, IIX, IIX1, IIY, IIY1, IIZ, IIZ1, IIAA, and IIAA1, R¹ is selected from:

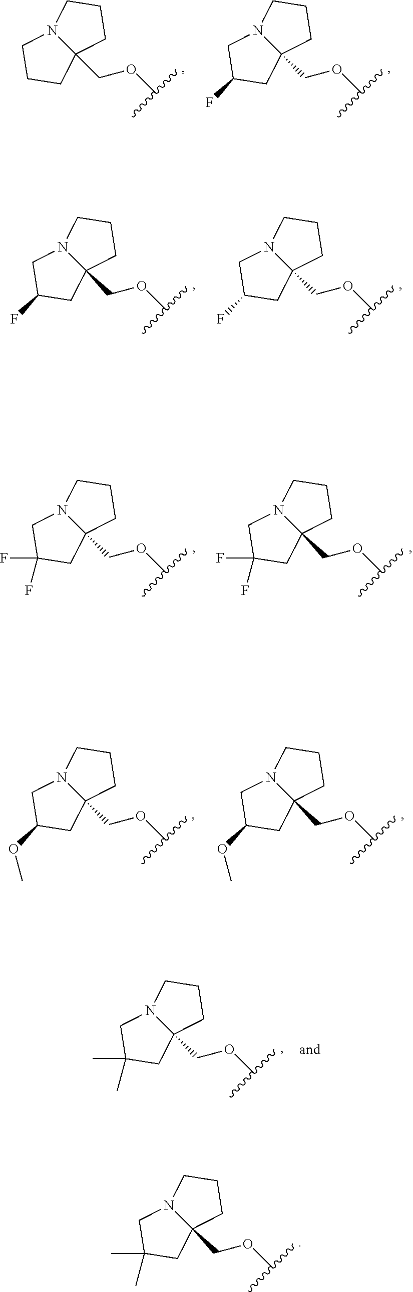

In some embodiments, for a compound of any one of Formulas II, I′, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, and IIAA1, R¹ is selected from:

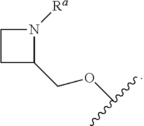

In some embodiments, R^{a} is C₁₋₆alkyl that is unsubstituted or substituted with one or more R¹³. In some embodiments, R^{a} is methyl. In some embodiments, R^{e} is selected from:

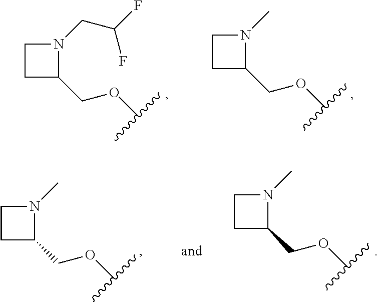

In some embodiments, for a compound of any one of Formulas II, II′, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, and IIAA1, R¹ is selected from:

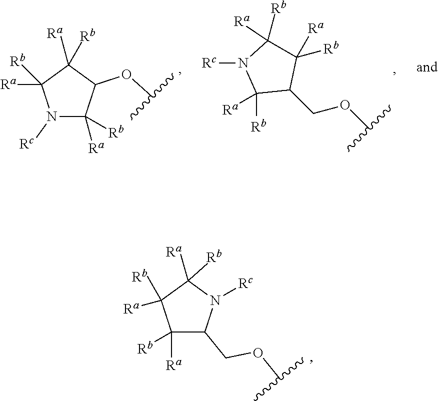

wherein each R^{a} and R^{b} is independently selected from halogen, C₁₋₆alkyl, —OR¹², and H; and R^{c} is selected from C₁₋₆alkyl, wherein an R^{a} and R^{b} or R^{c} optionally join together to form a 3-6 membered carbocycle or heterocycle, and wherein any C₁₋₆alkyl or 3-6 membered carbocycle or heterocycle is unsubstituted or is substituted with one or more R¹³. In some embodiments, each R^{a} and R^{b} is independently selected from halogen, C₁₋₆ alkyl, —OR¹², and H; and R^{c} is selected from C₁₋₆ alkyl, wherein any C₁₋₆ alkyl is unsubstituted or is substituted with one or more R¹³, and wherein an R^{a} and R^{b} attached to the same carbon atom join together to form a 3-6 membered carbocycle. In some embodiments, each R^{a} and R^{b} is independently selected from halogen, C₁₋₆ alkyl, —OR¹², and H; and wherein an R and R^{c} join together to form a 3-6 membered heterocycle. In some embodiments, each R and R is independently selected from halogen, C₁₋₆ alkyl, —OR¹², and H; and R^{e} is selected from C₁₋₆ alkyl, wherein any C₁₋₆ alkyl is unsubstituted or is substituted with one or more R¹³. In some embodiments, one R^{a} or R^{b} is selected from halogen, C₁₋₆alkyl, and —OR¹², and the other R^{a} and R^{b} groups are H. In some embodiments, one R^{a} or R^{b} is halogen (e.g., F). In some embodiments, two R^{a} groups, two R^{b} groups, or an R^{a} and an R^{b} are halogen (e.g., F). In some embodiments, one R^{a} or R^{b} is —OR¹² (e.g., —OCH₃ or —CHF₂). In some embodiments, one R^{a} or R^{b} is C₁₋₆ alkyl (e.g., methyl). In some embodiments, two R^{a} groups, two R^{b} groups, or an R′ and an R are C₁₋₆alkyl (e.g., methyl). In some embodiments, R^{e} is selected from —CH₃, —CH₂CH₂F, —CH₂CHF₂, and —CH₂CH₂CN. In some embodiments, an R^{a} and R^{b} join together to form a 3-6 membered carbocycle, such as a cyclopropane. In some embodiments, an R^{a} and R^{b} attached to the same carbon atom join together to form a 3-6 membered carbocycle, such as a cyclopropane. In some embodiments, an R and R join together to form a 3-6 membered heterocycle. In some embodiments, R^{e} is selected from:

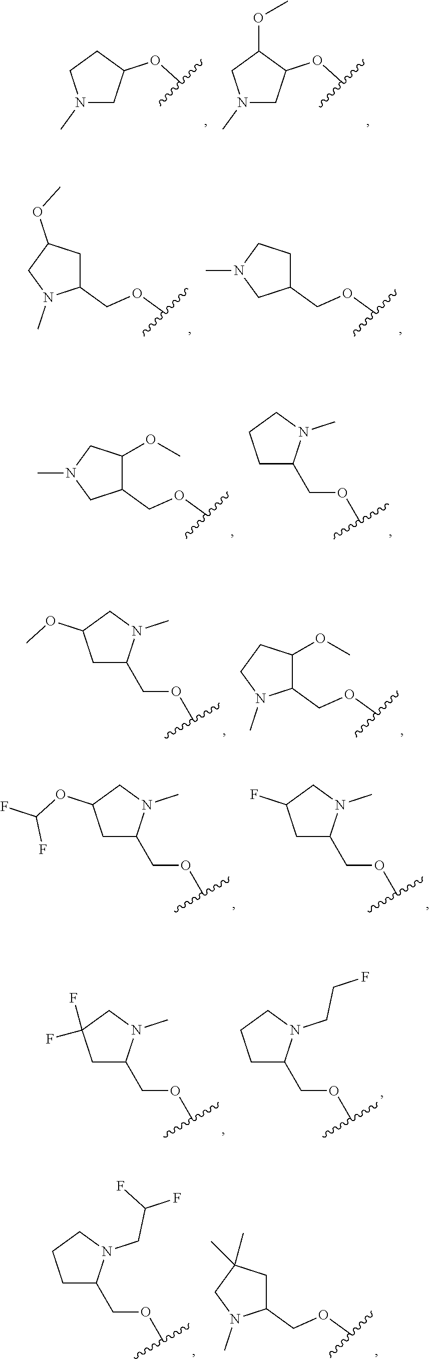

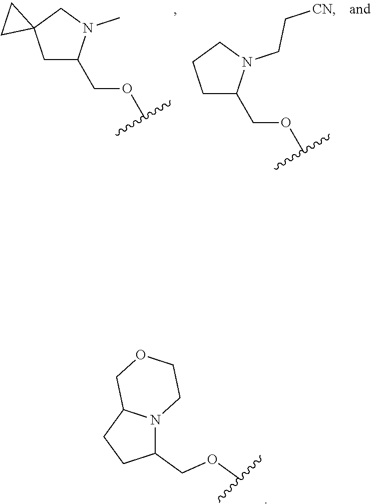 [SMILES: *OCC1CC2(CC2)CN1C ; *OCC1CCCN1CCC#N ; *OCC1CCC2COCCN12]

In some embodiments, for a compound of any one of Formulas II, II′, IIA, IIA1, IIB, IIB1, IC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, and IIAA1, R¹ is selected from:

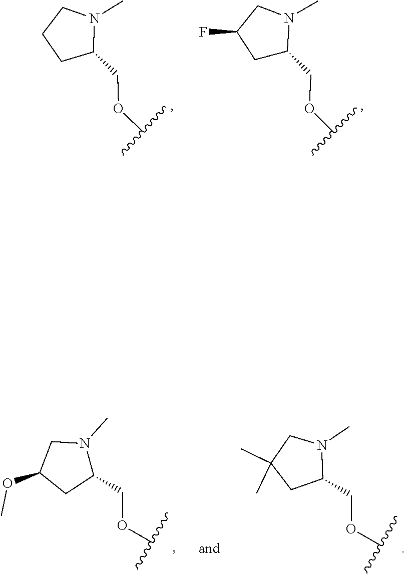

In some embodiments, for a compound of any one of Formulas II, II′, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, and IIAA1, R¹ is selected from:

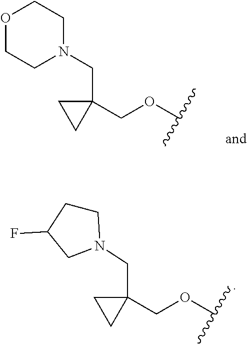 [SMILES: *OCC1(CN2CCOCC2)CC1 ; *OCC1(CN2CCC(F)C2)CC1]

In some embodiments, for a compound of any one of Formulas II, II′, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, and IIAA1, R⁵ is H. In some embodiments, R⁵ is a halogen (e.g., F or Cl). In some embodiments, R⁵ is C₁₋₆ In some embodiments, R⁵ is F. In some embodiments, R⁵ is —CN. In some embodiments, R⁵ is —OR¹², wherein R¹² is selected from C₁₋₆ alkyl and H. In some embodiments, R⁵ is —OCH₃. In some embodiments, R⁵ is —OCF₃. In some embodiments, R⁵ is selected from C₁₋₆alkyl that is unsubstituted or substituted with one or more R¹⁰. In some embodiments, R^{e} is selected from C₁₋₂alkyl that is unsubstituted or substituted with one or more R¹³. In some embodiments, R^{e} is selected from C₁₋₆alkyl that is unsubstituted, such as methyl or ethyl. In some embodiments, R⁵ is selected from C₁₋₆alkyl that is substituted with one or more halogens or —CN. In some embodiments, R⁵ is C₁₋₆alkyl that is substituted with one or more halogens, such as one or more fluorines. In some embodiments, R⁵ is —CF₃. In some embodiments, R⁵ is —CHF₂. In some embodiments, R⁵ is selected from —CF₂H, —CF₃, —CH₂CN, and —CH₂CH₃. In some embodiments, R⁵ is selected from —CH₃, —CH₂CH₃, —CF₂H, —CF₃, —CF₂CH₃, and —CH₂CN. In some embodiments, R⁵ is C₁₋₆alkyl that is substituted with one or more R¹³, wherein each R¹³ is independently selected from —OR¹⁴, —CN, and —N(R¹⁴)₂. In some embodiments, R⁵ is —CH₂CN. In some embodiments, R⁵ is a 3-6 membered heterocycle. In some embodiments, R⁵ is a 5-6 membered heteroaryl, such as a furan.

In some embodiments, for a compound of any one of Formulas II, II′, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, and IIAA1, R⁷ is C₁₋₆ In some embodiments, R⁷ is F.

In some embodiments, for a compound of any one of Formulas II, II′, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, and IIAA1, the compound is a not a compound included in Table 1, or a salt, ester, tautomer, prodrug, zwitterionic form, or stereoisomer(s) thereof.

TABLE 1

| Compound Number | Structure |
|---|---|
| II-67a | 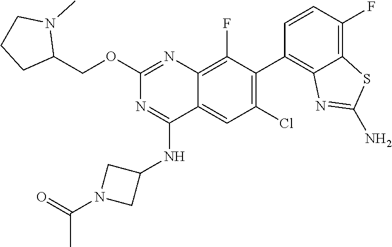 [SMILES: CC(=O)N1CC(Nc2nc(OCC3CCCN3C)nc3c(F)c(-c4ccc(F)c5sc(N)nc45)c(Cl)cc23)C1] |
| II-188 | 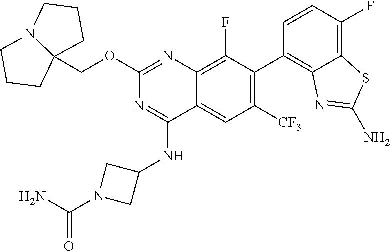 [SMILES: NC(=O)N1CC(Nc2nc(OCC34CCCN3CCC4)nc3c(F)c(-c4ccc(F)c5sc(N)nc45)c(C(F)(F)F)cc23)C1] |
| II-79a | 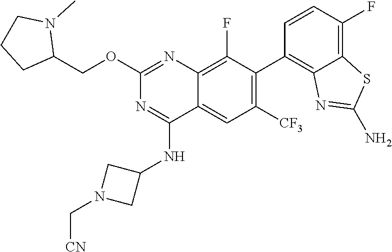 [SMILES: CN1CCCC1COc1nc(NC2CN(CC#N)C2)c2cc(C(F)(F)F)c(-c3ccc(F)c4sc(N)nc34)c(F)c2n1] |
| II-276 | 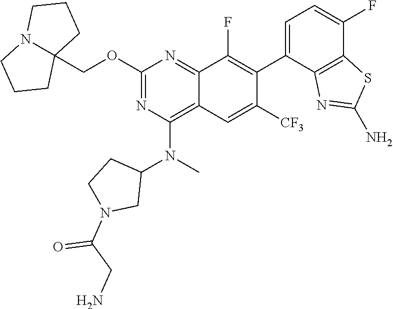 [SMILES: CN(c1nc(OCC23CCCN2CCC3)nc2c(F)c(-c3ccc(F)c4sc(N)nc34)c(C(F)(F)F)cc12)C1CCN(C(=O)CN)C1] |
| II-97a | 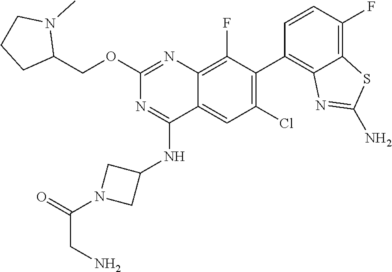 [SMILES: CN1CCCC1COc1nc(NC2CN(C(=O)CN)C2)c2cc(Cl)c(-c3ccc(F)c4sc(N)nc34)c(F)c2n1] |
| II-278 | 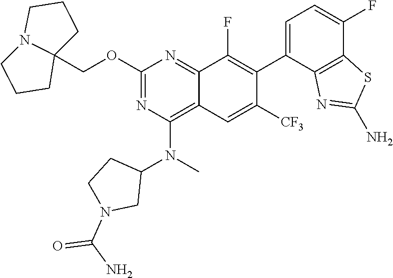 [SMILES: CN(c1nc(OCC23CCCN2CCC3)nc2c(F)c(-c3ccc(F)c4sc(N)nc34)c(C(F)(F)F)cc12)C1CCN(C(N)=O)C1] |
| II-114a | 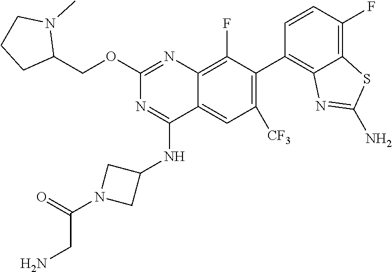 [SMILES: CN1CCCC1COc1nc(NC2CN(C(=O)CN)C2)c2cc(C(F)(F)F)c(-c3ccc(F)c4sc(N)nc34)c(F)c2n1] |
| II-130 | 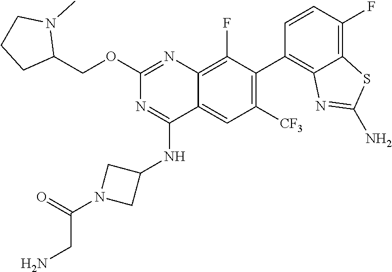 [SMILES: CN1CCCC1COc1nc(NC2CN(C(=O)CN)C2)c2cc(C(F)(F)F)c(-c3ccc(F)c4sc(N)nc34)c(F)c2n1] |
| II-163 | 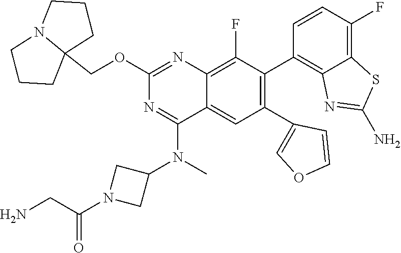 [SMILES: CN(c1nc(OCC23CCCN2CCC3)nc2c(F)c(-c3ccc(F)c4sc(N)nc34)c(-c3ccoc3)cc12)C1CN(C(=O)CN)C1] |
| II-137 | 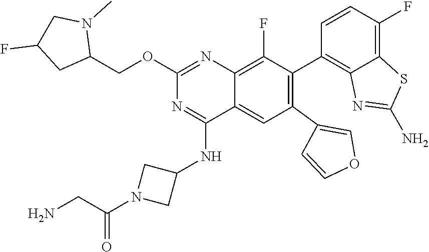 [SMILES: CN1CC(F)CC1COc1nc(NC2CN(C(=O)CN)C2)c2cc(-c3ccoc3)c(-c3ccc(F)c4sc(N)nc34)c(F)c2n1] |
| II-164 | 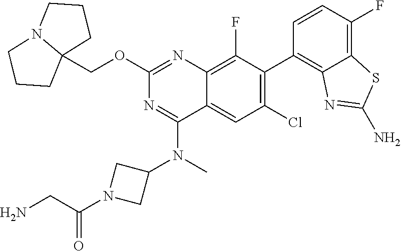 [SMILES: CN(c1nc(OCC23CCCN2CCC3)nc2c(F)c(-c3ccc(F)c4sc(N)nc34)c(Cl)cc12)C1CN(C(=O)CN)C1] |
| II-146 | 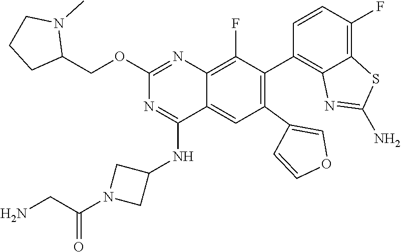 [SMILES: CN1CCCC1COc1nc(NC2CN(C(=O)CN)C2)c2cc(-c3ccoc3)c(-c3ccc(F)c4sc(N)nc34)c(F)c2n1] |
| II-167 | 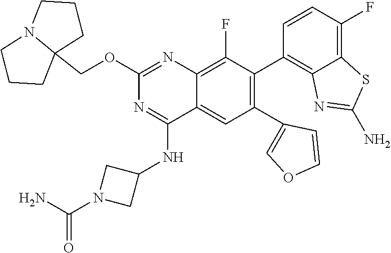 [SMILES: NC(=O)N1CC(Nc2nc(OCC34CCCN3CCC4)nc3c(F)c(-c4ccc(F)c5sc(N)nc45)c(-c4ccoc4)cc23)C1] |
| II-147 | 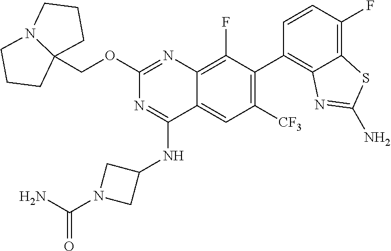 [SMILES: NC(=O)N1CC(Nc2nc(OCC34CCCN3CCC4)nc3c(F)c(-c4ccc(F)c5sc(N)nc45)c(C(F)(F)F)cc23)C1] |
| II-171 | 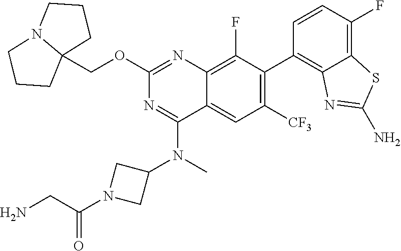 [SMILES: CN(c1nc(OCC23CCCN2CCC3)nc2c(F)c(-c3ccc(F)c4sc(N)nc34)c(C(F)(F)F)cc12)C1CN(C(=O)CN)C1] |
| II-150 | 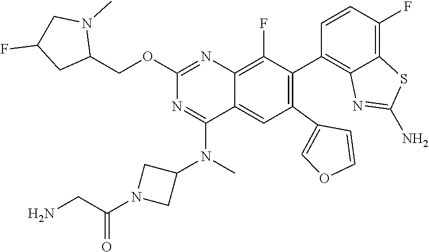 [SMILES: CN1CC(F)CC1COc1nc(N(C)C2CN(C(=O)CN)C2)c2cc(-c3ccoc3)c(-c3ccc(F)c4sc(N)nc34)c(F)c2n1] |
| II-173 | 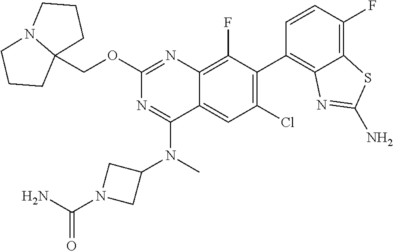 [SMILES: CN(c1nc(OCC23CCCN2CCC3)nc2c(F)c(-c3ccc(F)c4sc(N)nc34)c(Cl)cc12)C1CN(C(N)=O)C1] |
| II-156 | 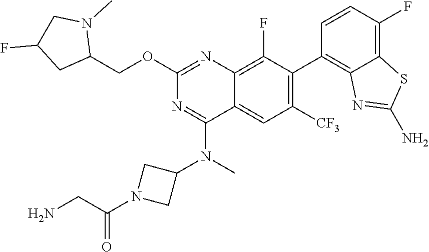 [SMILES: CN1CC(F)CC1COc1nc(N(C)C2CN(C(=O)CN)C2)c2cc(C(F)(F)F)c(-c3ccc(F)c4sc(N)nc34)c(F)c2n1] |
| II-182 | 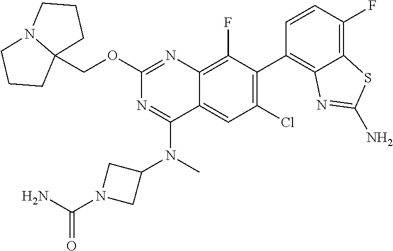 [SMILES: CN(c1nc(OCC23CCCN2CCC3)nc2c(F)c(-c3ccc(F)c4sc(N)nc34)c(Cl)cc12)C1CN(C(N)=O)C1] |
| II-183 | 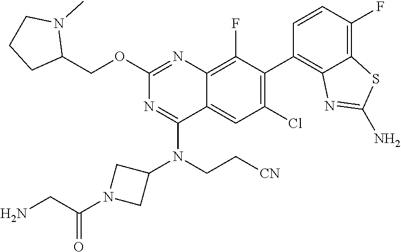 [SMILES: CN1CCCC1COc1nc(N(CCC#N)C2CN(C(=O)CN)C2)c2cc(Cl)c(-c3ccc(F)c4sc(N)nc34)c(F)c2n1] |
| II-187 | 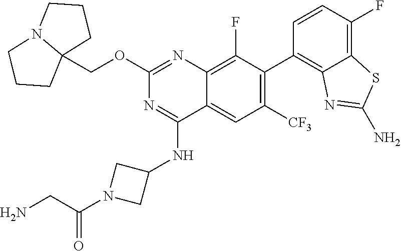 [SMILES: NCC(=O)N1CC(Nc2nc(OCC34CCCN3CCC4)nc3c(F)c(-c4ccc(F)c5sc(N)nc45)c(C(F)(F)F)cc23)C1] |
| II-197 | 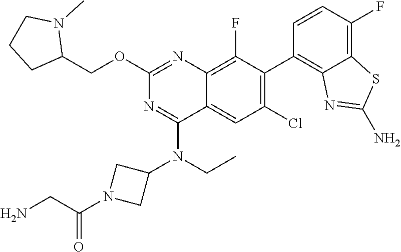 [SMILES: CCN(c1nc(OCC2CCCN2C)nc2c(F)c(-c3ccc(F)c4sc(N)nc34)c(Cl)cc12)C1CN(C(=O)CN)C1] |
| II-151 | 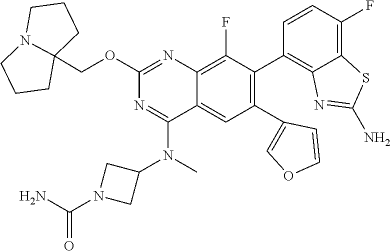 [SMILES: CN(c1nc(OCC23CCCN2CCC3)nc2c(F)c(-c3ccc(F)c4sc(N)nc34)c(-c3ccoc3)cc12)C1CN(C(N)=O)C1] |
| II-230a | 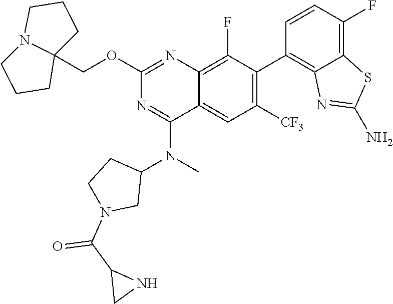 [SMILES: CN(c1nc(OCC23CCCN2CCC3)nc2c(F)c(-c3ccc(F)c4sc(N)nc34)c(C(F)(F)F)cc12)C1CCN(C(=O)C2CN2)C1] |
| II-235 | 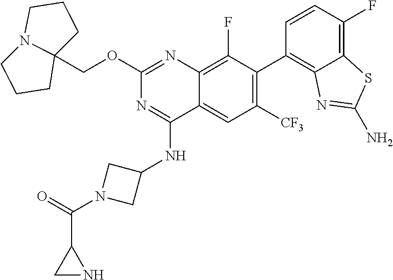 [SMILES: Nc1nc2c(-c3c(C(F)(F)F)cc4c(NC5CN(C(=O)C6CN6)C5)nc(OCC56CCCN5CCC6)nc4c3F)ccc(F)c2s1] |

In another aspect, the present disclosure provides a compound represented by Formula III′:

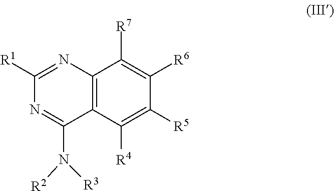

or a salt (e.g., pharmaceutically acceptable salt), ester, tautomer, prodrug, zwitterionic form, or stereoisomer(s) thereof, wherein:
R¹ is selected from —OR⁸ and

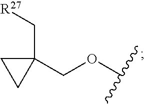

R² and R³, together with the nitrogen atom to which they are attached, form a 4-10 membered heterocycle that is unsubstituted or is substituted with one or more R¹¹;
R⁴ is H;
R⁵ is selected from halogen and C₁₋₆alkyl, wherein any C₁₋₆alkyl is unsubstituted or substituted with one or more R¹³;
R^{e} is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹¹ is independently selected from deuterium, —OR¹², ═O, =N(R¹⁴), —C(O)(C₁₋₆alkylene)CN, —C(O)(C₁₋₆alkylene)OR¹⁴, —C(O)(C₁₋₆alkyl), —C(O)O(C₁₋₆alkyl), —C(O)N(R¹⁴)₂, —C(O)(3-6 membered carbocycle or heterocycle), —S(O)₂(C₁₋₆alkyl), —N(R¹⁴)C(O)(C₁₋₆alkyl), —N(R¹²)C(O)(C₁₋₆alkylene)OR¹⁴, halogen, —CN, a 3-6 membered carbocycle or heterocycle, a 5-6 membered heteroaryl, and C₁₋₆alkyl, wherein any C₁₋₆alkyl, carbocycle, heterocycle, or heteroaryl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹³;
each R²⁰ is independently selected from —OH, —OC₄alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen;
R^{a} and R^{b} are each independently selected from deuterium, halogen, C₁₋₆alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.

In some embodiments, the present disclosure provides a compound of Formula III′ or a salt (e.g., a pharmaceutically acceptable salt) thereof.

In another aspect, the present disclosure provides a compound represented by Formula III:

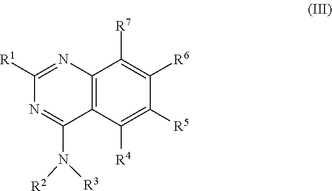

or a salt (e.g., pharmaceutically acceptable salt), ester, tautomer, prodrug, zwitterionic form, or steroisomer(s) thereof, wherein:
R¹ is selected from —OR⁸ and

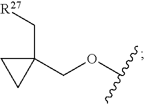

R² and R³, together with the nitrogen atom to which they are attached, form a 4-10 membered heterocycle that is unsubstituted or is substituted with one or more R¹¹, provided that (i) when the heterocycle contains an additional nitrogen atom, the additional nitrogen atom is substituted with R¹¹ or (ii) the heterocycle does not comprise an —NH— moiety;
R⁴ is H;
R⁵ is selected from halogen and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹¹ is independently selected from deuterium, —OR¹², ═O, =N(R¹²), —C(O)(C₁₋₆alkylene)CN, —C(O)(C₁₋₆alkylene)OR¹⁴, —C(O)(C₁₋₆alkyl), —C(O)O(C₁₋₆alkyl), —C(O)N(R¹²)₂, —C(O)(3-6 membered carbocycle or heterocycle), —S(O)₂(C₁₋₆alkyl), —N(R¹⁴)C(O)(C₁₋₆alkyl), —N(R¹⁴)C(O)C₁₋₆alkylene)OR¹⁴, halogen, —CN, a 3-6 membered carbocycle or heterocycle, a 5-6 membered heteroaryl, and C₁₋₆alkyl, wherein any C₁₋₆alkyl, carbocycle, heterocycle, or heteroaryl is unsubstituted or substituted with one or more R¹³;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹³;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R¹³;
each R²⁸ is independently selected from C₁₋₆alkyl and halogen;
R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.

In some embodiments, the present disclosure provides a compound of Formula III or a salt (e.g., a pharmaceutically acceptable salt) thereof.

In some embodiments, the present disclosure provides a compound of Formula III, wherein:
R¹ is selected from —OR⁸;
R² and R³, together with the nitrogen atom to which they are attached, form a 4-10 membered heterocycle that is unsubstituted or is substituted with one or more R¹¹, provided that (i) when the heterocycle contains an additional nitrogen atom, the additional nitrogen atom is substituted with R¹¹ or (ii) the heterocycle does not comprise an —NH— moiety;
R⁴ is H;
R⁵ is C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen;
R⁸ is an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹¹ is independently selected from deuterium, —OR¹⁴, ═O, =N(R¹⁴), —C(O)(C₁₋₆alkylene)CN, —C(O)(C₁₋₆alkylene)OR¹⁴, —C(O)(C₁₋₆alkyl), —C(O)O(C₁₋₆alkyl), —C(O)N(R¹⁴)₂, —C(O)(3-6 membered carbocycle or heterocycle), —S(O)₂(C₁₋₆alkyl), —N(R¹⁴)C(O)(C₁₋₆alkyl), —N(R¹⁴)C(O)(C₁₋₆alkylene)OR¹⁴, halogen, —CN, a 3-6 membered carbocycle or heterocycle, a 5-6 membered heteroaryl, and C₁₋₆alkyl, wherein any C₁₋₆alkyl, carbocycle, heterocycle, or heteroaryl is unsubstituted or substituted with one or more R²⁰;
each R¹² is independently selected from C₁₋₆alkyl and H, wherein any C₁₋₆alkyl is unsubstituted or substituted with one or more R¹³;
each R¹³ is independently selected from halogen;
each R¹⁴ is independently selected from C₁₋₆ alkyl and H;
each R¹⁵ is independently selected from deuterium, halogen, —N(R¹²)₂, —CN, and C₁₋₆alkyl, wherein any C₁₋₆alkyl is unsubstituted or substituted with one or more R¹³;
each R²⁰ is independently selected from —OH, —OC₁₋₆alkyl, —CN, and halogen;
R^{a} and R^{b} are each independently selected from deuterium, halogen, C₁₋₆ alkyl, and H, wherein an R^{a} and R^{b} optionally join together to form a 3-6 membered carbocycle, and wherein any C₁₋₆alkyl or 3-6 membered carbocycle is unsubstituted or is substituted with one or more R¹³.

In some embodiments, the present disclosure provides a compound of Formula III′ or III, wherein the compound is of Formula III-a:

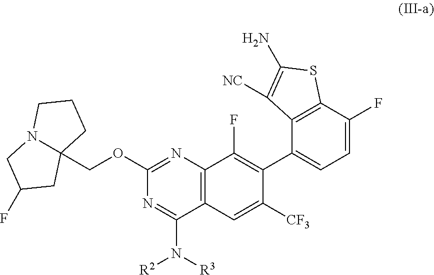

or a salt (e.g., pharmaceutically acceptable salt), ester, tautomer, prodrug, zwitterionic form, or stereoisomer(s) thereof, wherein R² and R³ are as defined for Formula III above and described in classes and subclasses herein, both singly and in combination. In some embodiments, R² and R³ are as defined for Formula III′ above and described in classes and subclasses herein, both singly and in combination. In some embodiments, the present disclosure provides a compound of Formula III-a, or a salt (e.g., pharmaceutically acceptable salt) thereof.

In some embodiments, for a compound according to Formula III, III′, or III-a, R² and R³, together with the nitrogen atom to which they are attached, form a 4-7 membered heterocycle that is unsubstituted or is substituted with one or more R¹¹. In some embodiments, R² and R³, together with the nitrogen atom to which they are attached, form a 4-7 membered heterocycle that is unsubstituted or is substituted with one or more R¹¹, provided that (i) when the heterocycle contains an additional nitrogen atom, the additional nitrogen atom is substituted with R¹¹ or (ii) the heterocycle does not comprise an —NH— moiety. In some embodiments, R² and R³, together with the nitrogen atom to which they are attached, form a 4-7 membered heterocycle that is unsubstituted or is substituted with one or more R¹¹, provided that (i) when the heterocycle contains an additional nitrogen atom, the additional nitrogen atom is substituted with R¹¹ or (ii) when the heterocycle comprises a single ring, the heterocycle does not comprise an —NH— moiety. In some embodiments, R² and R³, together with the nitrogen atom to which they are attached, form a 4-7 membered heterocycle comprising a single ring, wherein the heterocycle is unsubstituted or is substituted with one or more R¹¹, provided that the heterocycle does not comprise an —NH— moiety. In some embodiments, R² and R³, together with the nitrogen atom to which they are attached, form a 4-6 membered heterocycle that is unsubstituted or is substituted with one or more R¹³. In some embodiments, R² and R³, together with the nitrogen atom to which they are attached, form a 4-6 membered heterocycle that is unsubstituted or is substituted with one or more R¹³, provided that (i) when the heterocycle contains an additional nitrogen atom, the additional nitrogen atom is substituted with R¹¹ or (ii) the heterocycle does not comprise an —NH-moiety. In some embodiments, R² and R³, together with the nitrogen atom to which they are attached, form a 4-6 membered heterocycle that is unsubstituted or is substituted with one or more R¹¹, provided that (i) when the heterocycle contains an additional nitrogen atom, the additional nitrogen atom is substituted with R¹¹ or (ii) when the heterocycle comprises a single ring, the heterocycle does not comprise an —NH— moiety. In some embodiments, R² and R³, together with the nitrogen atom to which they are attached, form a 4-6 membered heterocycle that is unsubstituted, provided that the 4-6 membered heterocycle does not contain an —NH— moiety. In some embodiments, R² and R³, together with the nitrogen atom to which they are attached, form a 7-membered heterocycle that is unsubstituted, provided that the 7-membered heterocycle does not contain an —NH— moiety. In some embodiments, R² and R³, together with the nitrogen atom to which they are attached, form a 4-6 membered heterocycle that is substituted with one or more R¹¹, provided that (i) when the heterocycle contains an additional nitrogen atom, the additional nitrogen atom is substituted with R¹¹ or (ii) the heterocycle does not comprise an —NH— moiety. In some embodiments, R² and R³, together with the nitrogen atom to which they are attached, form a 4-6 membered heterocycle that includes 1 or 2 heteroatoms selected from O, S, and N, wherein the heterocycle is unsubstituted or is substituted with one or more R¹¹, provided that (i) when the heterocycle contains an additional nitrogen atom, the additional nitrogen atom is substituted with R¹¹ or (ii) the heterocycle does not comprise an —NH-moiety. In some embodiments, R² and R³, together with the nitrogen atom to which they are attached, form a 4-6 membered heterocycle that includes 1 or 2 heteroatoms selected from O, S, and N, wherein the heterocycle is substituted with 1-4 R¹¹, provided that (i) when the heterocycle contains an additional nitrogen atom, the additional nitrogen atom is substituted with R¹¹ or (ii) the heterocycle docs not comprise an —NH— moiety. In some embodiments, R² and R³, together with the nitrogen atom to which they are attached, form a pyrrolidine that is substituted with 0-4 R¹¹. In some embodiments, R² and R³, together with the nitrogen atom to which they are attached, form an azetidine that is substituted with 0-4 R¹¹. In some embodiments, R² and R³, together with the nitrogen atom to which they are attached, form a piperidine that is substituted with 0-4 R¹¹. In some embodiments, R² and R³, together with the nitrogen atom to which they are attached, form a piperazine that is substituted with 0-4 R¹¹, provided that the additional nitrogen atom of the piperazine is substituted with R¹¹. In some embodiments, R² and R³, together with the nitrogen atom to which they are attached, form a morpholine or thiomorpholine that is substituted with 0-4 R¹¹. In some embodiments, R² and R³, together with the nitrogen atom to which they are attached, form a 4-6 membered heterocycle substituted with one or more R¹¹, provided that (i) when the heterocycle contains an additional nitrogen atom, the additional nitrogen atom is substituted with R¹¹ or (ii) the heterocycle does not comprise an —NH— moiety, wherein at least one R¹⁴ is selected from —OR¹² and a C₁₋₆alkyl substituted with —OH. In some embodiments, R² and R³, together with the nitrogen atom to which they are attached, form a 4-6 membered heterocycle substituted with one or more R¹¹, provided that (i) when the heterocycle contains an additional nitrogen atom, the additional nitrogen atom is substituted with R¹¹ or (ii) the heterocycle does not comprise an —NH— moiety, wherein at least one R¹⁴ is an unsubstituted C₁₋₆alkyl. In some embodiments, R² and R³, together with the nitrogen atom to which they are attached, form a 7-membered heterocycle that includes 1-3 heteroatoms selected from O, S, and N, wherein the heterocycle is unsubstituted or is substituted with one or more R¹¹. In some embodiments, R² and R³, together with the nitrogen atom to which they are attached, form a 7-membered heterocycle that includes 1-3 heteroatoms selected from O, S, and N, wherein the heterocycle is unsubstituted or is substituted with one or more R¹¹, provided that (i) when the heterocycle contains an additional nitrogen atom, the additional nitrogen atom is substituted with R¹¹ or (ii) the heterocycle does not comprise an —NH— moiety. In some embodiments, R² and R³, together with the nitrogen atom to which they are attached, form a 7-membered heterocycle that includes 1-3 heteroatoms selected from O, S, and N, wherein the heterocycle is substituted with 1-4 R¹¹. In some embodiments, R² and R³, together with the nitrogen atom to which they are attached, form a 7-membered heterocycle that includes 1-3 heteroatoms selected from O, S, and N, wherein the heterocycle is substituted with 1-4 R¹¹, provided that (i) when the heterocycle contains an additional nitrogen atom, the additional nitrogen atom is substituted with R¹¹ or (ii) the heterocycle does not comprise an —NH— moiety. In some embodiments, R² and R³, together with the nitrogen atom to which they are attached, form a 7-membered heterocycle that includes a nitrogen atom. In some embodiments, R² and R³, together with the nitrogen atom to which they are attached, form a 7-membered heterocycle that includes a nitrogen atom and an oxygen atom. In some embodiments, R² and R³, together with the nitrogen atom to which they are attached, form a 7-membered heterocycle that includes a nitrogen atom and a sulfur atom, which sulfur atom is optionally substituted with two oxo moieties. In some embodiments, R² and R³, together with the nitrogen atom to which they are attached, form a 7-membered heterocycle that includes two nitrogen atoms. In some embodiments, R² and R³, together with the nitrogen atom to which they are attached, form a 7-membered heterocycle that includes one additional nitrogen atom, provided that the additional nitrogen atom is substituted with R¹¹.

In some embodiments, for a compound according to Formula III, III′, or III-a, R² and R³, together with the nitrogen atom to which they are attached, form a bridged heterocycle that is unsubstituted or is substituted with one or more R¹¹. In some embodiments, for a compound according to Formula III, III′, or III-a, R² and R³, together with the nitrogen atom to which they are attached, form a bridged heterocycle that is unsubstituted or is substituted with one or more R¹¹, provided that (i) when the heterocycle contains an additional nitrogen atom, the additional nitrogen atom is substituted with R¹¹ or (ii) the heterocycle does not comprise an —NH— moiety. In some embodiments, R² and R³, together with the nitrogen atom to which they are attached, form a 7-9 membered bridged heterocycle that includes 1 or 2 heteroatoms selected from O, S, and N, wherein the bridged heterocycle is unsubstituted or is substituted with one or more R¹¹, provided that (i) when the heterocycle contains an additional nitrogen atom, the additional nitrogen atom is substituted with R¹¹ or (ii) the heterocycle does not comprise an —NH— moiety. In some embodiments, R² and R³, together with the nitrogen atom to which they are attached, form a 7-9 membered bridged heterocycle that includes 1 or 2 heteroatoms selected from O, S, and N, wherein the bridged heterocycle is substituted with 1-4 R¹¹, provided that (i) when the heterocycle contains an additional nitrogen atom, the additional nitrogen atom is substituted with R¹¹ or (ii) the heterocycle does not comprise an —NH— moiety. In some embodiments, R² and R³, together with the nitrogen atom to which they are attached, form a bridged piperidine that is substituted with 0-4 R¹¹. In some embodiments, R² and R³, together with the nitrogen atom to which they are attached, form a bridged piperazine that is substituted with 0-4 R¹¹, provided that (i) the additional nitrogen atom of the piperazine is substituted with R¹¹ or (ii) the bridged piperazine does not comprise an —NH— moiety. In some embodiments, R² and R³, together with the nitrogen atom to which they are attached, form a bridged morpholine or thiomorpholine that is substituted with 0-4 R¹¹. In some embodiments, R² and R³, together with the nitrogen atom to which they are attached, form a bridged heterocycle substituted with one or more R¹¹, provided that (i) when the heterocycle contains an additional nitrogen atom, the additional nitrogen atom is substituted with R¹¹ or (ii) the heterocycle does not comprise an —NH— moiety, wherein at least one R¹⁴ is selected from —OR² and a C₁₋₆alkyl substituted with —OH. In some embodiments, R² and R³, together with the nitrogen atom to which they are attached, form a bridged heterocycle substituted with one or more R¹¹, provided that (i) when the heterocycle contains an additional nitrogen atom, the additional nitrogen atom is substituted with R¹¹ or (ii) the heterocycle docs not comprise an —NH— moiety, wherein at least one R¹⁴ is an unsubstituted C₁₋₆alkyl.

In some embodiments, for a compound according to Formula III, III′, or III-a, R² and R³, together with the nitrogen atom to which they are attached, form a spirocycle that is unsubstituted or is substituted with one or more R¹¹. In some embodiments, for a compound according to Formula III, III′, or III-a, R² and R³, together with the nitrogen atom to which they are attached, form a spirocycle that is unsubstituted or is substituted with one or more R¹¹, provided that (i) when the spirocycle contains an additional nitrogen atom, the additional nitrogen atom is substituted with R¹¹ or (ii) the spirocycle does not comprise an —NH-moiety. In some embodiments, R² and R³, together with the nitrogen atom to which they are attached, form a spirocycle that is unsubstituted, provided that the spirocycle does not include an —NH— moiety. In some embodiments, R² and R³, together with the atom to which they are attached, form a spirocycle that is substituted with one or more R¹¹, provided that (i) when the spirocycle contains an additional nitrogen atom, the additional nitrogen atom is substituted with R¹¹ or (ii) the spirocycle docs not comprise an —NH— moiety. In some embodiments, R² and R³, together with the nitrogen atom to which they are attached, form a spirocycle comprising a 4-membered ring and a 3-membered ring. In some embodiments, R² and R³, together with the nitrogen atom to which they are attached, form a spirocycle comprising two 4-membered rings. In some embodiments, R² and R³, together with the nitrogen atom to which they are attached, form a spirocycle comprising a 4-membered ring and a 5-membered ring. In some embodiments, R² and R³, together with the nitrogen atom to which they are attached, form a spirocycle comprising a 4-membered ring and a 6-membered ring. In some embodiments, R² and R³, together with the nitrogen atom to which they are attached, form a spirocycle comprising a 6-membered ring and a 5-membered ring. In some embodiments, R² and R³, together with the nitrogen atom to which they are attached, form a spirocycle comprising two 5-membered rings.

In some embodiments, for a compound according to Formula III, III′, or III-a, R² and R³, together with the nitrogen atom to which they are attached, form a fused ring system including at least two rings, wherein the fused ring system is unsubstituted or is substituted with one or more R¹¹. In some embodiments, R² and R³, together with the nitrogen atom to which they are attached, form a fused ring system including at least two rings, wherein the fused ring system is unsubstituted or is substituted with one or more R¹¹, and provided that the fused ring system does not comprise an —NH— moiety. In some embodiments, R² and R³, together with the nitrogen atom to which they are attached, form a fused ring system including at least two rings, wherein at least one of the rings is selected from azetidine, pyrrolidine, piperidine, piperazine, triazole (e.g., 1,2,3-triazole or 1,2,4-triazole), pyrazole, pyrrole, imidazole, isoxazole, thiazole, oxazolidine, morpholine, tetrahydrofuran, azepane, and diazepane, and wherein the fused ring system is unsubstituted or is substituted with one or more R¹¹. In some embodiments, R² and R³, together with the nitrogen atom to which they are attached, form a fused ring system including at least two rings, wherein at least one of the rings is selected from azetidine, pyrrolidine, piperidine, piperazine, triazole (e.g., 1,2,3-triazole or 1,2,4-triazole), pyrazole, pyrrole, imidazole, isoxazole, thiazole, oxazolidine, morpholine, pyridine, tetrahydrofuran, azepane, and diazepane, and wherein the fused ring system is unsubstituted or is substituted with one or more R¹¹. In some embodiments, R² and R³, together with the nitrogen atom to which they are attached, form a fused ring system comprising two 5-membered rings. In some embodiments, R² and R³, together with the nitrogen atom to which they are attached, form a fused ring system comprising a 5-membered ring and a 6-membered ring. In some embodiments, R² and R³, together with the nitrogen atom to which they are attached, form a fused ring system comprising a 5-membered ring and a 4-membered ring. In some embodiments, R² and R³, together with the nitrogen atom to which they are attached, form a fused ring system comprising a 6-membered ring and a 3-membered ring. In some embodiments, R² and R³, together with the nitrogen atom to which they are attached, form a fused ring system comprising a 7-membered ring and a 5-membered ring. In some embodiments, R² and R³, together with the nitrogen atom to which they are attached, form a fused ring system including at least two rings, wherein the fused ring system is unsubstituted. In some embodiments, R² and R³, together with the nitrogen atom to which they are attached, form a fused ring system including at least two rings, wherein the fused ring system is unsubstituted, provided that the fused ring system does not comprise an —NH— moiety. In some embodiments, R² and R³, together with the nitrogen atom to which they are attached, form a fused ring system including at least two rings, wherein the fused ring system is substituted with one or more R¹¹. In some embodiments, R² and R³, together with the nitrogen atom to which they are attached, form a fused ring system including at least two rings, wherein the fused ring system is substituted with one or more R¹¹, provided that (i) when the fused ring system contains an additional nitrogen atom, the additional nitrogen atom is substituted with R¹¹ or (ii) the fused ring system does not comprise an —NH— moiety. In some embodiments, R² and R³, together with the nitrogen atom to which they are attached, form a fused ring system including at least two rings, wherein the fused ring system is substituted with 1-4 R¹¹. In some embodiments, R² and R³, together with the nitrogen atom to which they are attached, form a fused ring system including at least two rings, wherein the fused ring system is substituted with 1-4 R¹¹, provided that (i) when the fused ring system contains an additional nitrogen atom, the additional nitrogen atom is substituted with R¹¹ or (ii) the fused ring system does not comprise an —NH— moiety.

In some embodiments, for a compound according to Formula III, III′, or III-a, R² and R³, together with the nitrogen atom to which they are attached, form a 4-10 membered heterocycle that is unsubstituted or is substituted with one or more R¹¹, wherein each R¹⁵ is independently selected from deuterium, —OR¹², ═O, ═N(R¹⁴), —C(O)(C₁₋₆alkylene)CN, —C(O)(C₁₋₆alkylene)OR¹⁴, —C(O)(C₁₋₆alkyl), —C(O)N(R¹²)₂, —C(O)(3-6 membered carbocycle or heterocycle), —S(O)₂(C₁₋₆akyl), —N(R¹²)C(O)(C₁₋₆alkyl), —N(R¹²)C(O)(C₁₋₆ alkylene)OR¹⁴, halogen, —CN, a 3-6 membered carbocycle or heterocycle, and C₁₋₆alkyl, wherein any C₁₋₆ alkyl is unsubstituted or substituted with one or more R²⁰. In some embodiments, each R¹¹ is independently selected from deuterium, —OR¹², —O, ═N(R¹⁴), —C(O)(C₁₋₆alkylene)CN, —C(O)(C₁₋₆alkylene)OR¹⁴, —C(O)(C₁₋₆ alkyl), —C(O)N(R¹⁴)₂, —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰. In some embodiments, each R¹¹ is independently selected from —OR¹², —C(O)C₁₋₆alkylene)CN, —C(O)(C₁₋₆alkylene)OR¹⁴, —C(O)(C₁₋₆alkyl), —C(O)N(R¹²)₂, —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R¹¹. In some embodiments, each R¹⁵ is independently selected from deuterium, —OR¹⁴, —C(O)(C₁₋₆alkylene)CN, —C(O)(C₁₋₆alkylene)OR¹⁴, —C(O)(C₁₋₆alkyl), —C(O)N(R¹²)₂, halogen, and C₁₋₆alkyl, wherein any C₁₋₆alkyl is unsubstituted or substituted with one or more R²⁰. In some embodiments, at least one R¹⁴ is —C(O)(3-6 membered carbocycle or heterocycle), wherein any carbocycle or heterocycle is unsubstituted or substituted with one or more R²⁰. In some embodiments, at least one R¹⁴ is —C(O)(3-6 membered carbocycle), wherein the carbocycle is unsubstituted or substituted with one or more R²⁰. In some embodiments, at least one R¹⁴ is —C(O)(3-6 membered heterocycle), wherein the heterocycle is unsubstituted or substituted with one or more R²⁰. In some embodiments, at least one R²³ is —C(O)N(R¹⁴)₂. In some embodiments, at least one R¹⁴ is ═O. In some embodiments, when R²³ is —C(O)N(R¹⁴)₂, then at least one R¹⁴ is not H. In some embodiments, when R²³ is —C(O)N(R¹²)₂, then each R¹⁴ is C₁₋₆ alkyl (e.g., methyl or ethyl).

In some embodiments, for a compound according to Formula III, III′, or III-a, R² and R³, together with the nitrogen atom to which they are attached, form a 4-10 membered heterocycle that is unsubstituted or is substituted with one or more R¹¹, provided that when the heterocycle contains an additional nitrogen atom, the additional nitrogen atom is substituted with R¹¹, wherein the one or more R¹¹ are independently selected from —OR¹², ═O, —C(O(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹²)C(O)(C₁₋₆alkylene)OR¹⁴, halogen, and C₁₋₆alkyl that is unsubstituted or substituted with one or more R²⁰. In some embodiments, R² and R³, together with the nitrogen atom to which they are attached, form a 4-10 membered heterocycle that is unsubstituted or is substituted with one or more R¹¹, provided that (i) when the heterocycle contains an additional nitrogen atom, the additional nitrogen atom is substituted with R¹¹ or (ii) the heterocycle does not comprise an —NH-moiety, wherein the one or more R¹¹ are independently selected from deuterium, —OR¹², ═O, ═N(R¹²), —C(O)(C₁₋₆alkylene)CN, —C(O)(C₁₋₆alkylene)OR¹⁴, —C(O)(C₁₋₆alkyl), —C(O)(3-6 membered carbocycle or heterocycle), —S(O)₂(C₁₋₆alkyl), —N(R¹²)C(O)(C₁₋₆alkyl), —N(R¹⁴)C(O)(C₁₋₆alkylene)OR¹⁴, halogen, —CN, a 3-6 membered carbocycle or heterocycle, and C₁₋₆alkyl that is unsubstituted or substituted with one or more R². In some embodiments, R² and R³, together with the nitrogen atom to which they are attached, form a 4-10 membered heterocycle that is unsubstituted or is substituted with one or more R¹¹, provided that (i) when the heterocycle contains an additional nitrogen atom, the additional nitrogen atom is substituted with R¹¹ or (ii) the heterocycle does not comprise an —NH— moiety, wherein the one or more R¹¹ are independently selected from deuterium, —OR¹², ═O, ═N(R¹⁴), —C(O)(C₁₋₆alkylene)CN, —C(O)(C₁₋₆alkylene)OR¹⁴, —C(O)(C₁₋₆ alkyl), —C(O)O(C₁₋₆alkyl), —C(O)(3-6 membered carbocycle or heterocycle), —S(O)₂(C₁₋₆alkyl), —N(R¹⁴)C(O)(C₁₋₆alkyl), —N(R¹⁴)C(O)(C₁₋₆alkylene)OR¹⁴, halogen, —CN, a 3-6 membered carbocycle or heterocycle, a 5-6 membered heteroaryl, and C₁₋₆alkyl that is unsubstituted or substituted with one or more R²⁰.

In some embodiments, for a compound according to Formula III, III′, or III-a, R² and R³, together with the nitrogen atom to which they are attached, form a structure selected from:

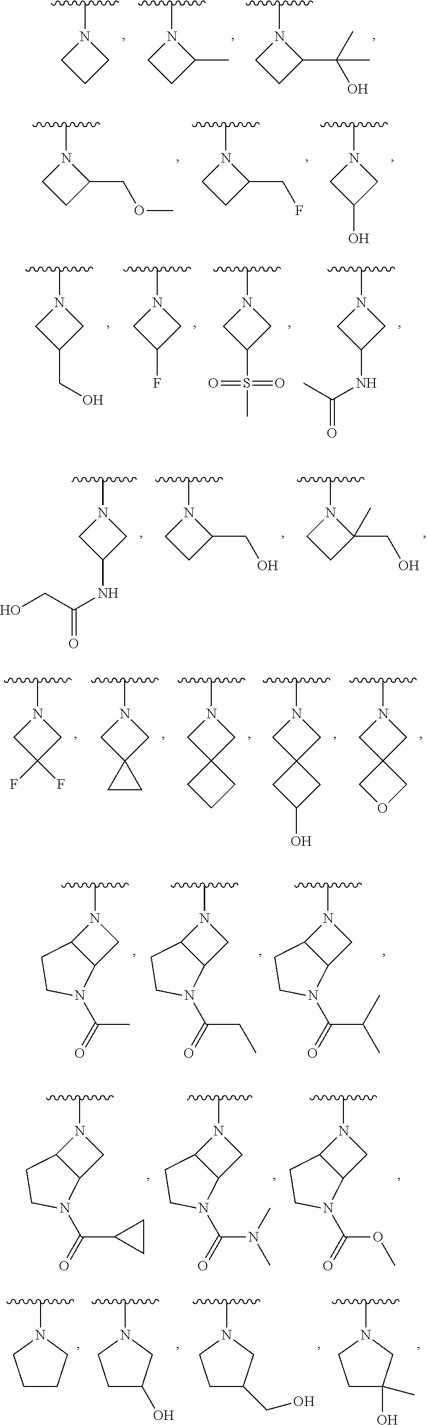

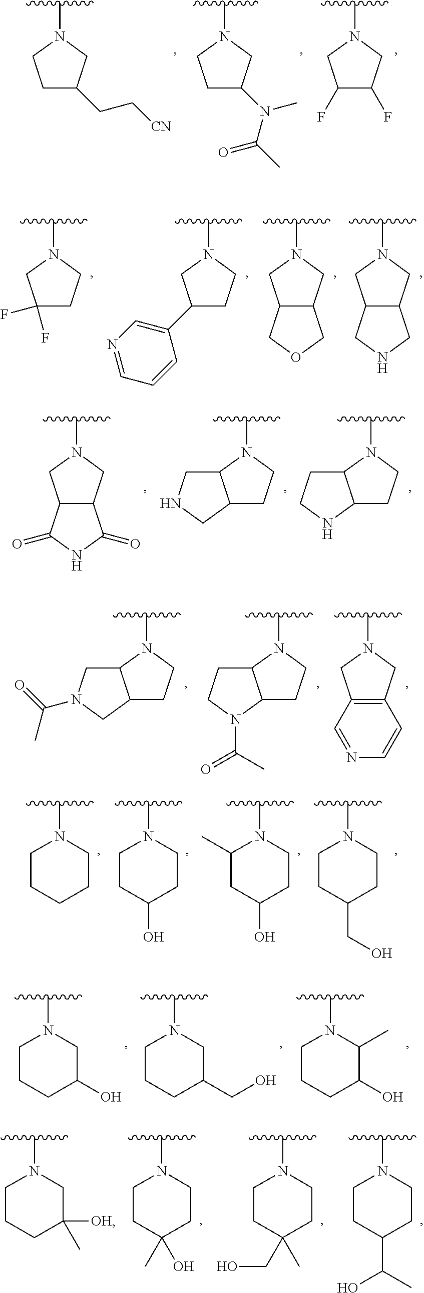

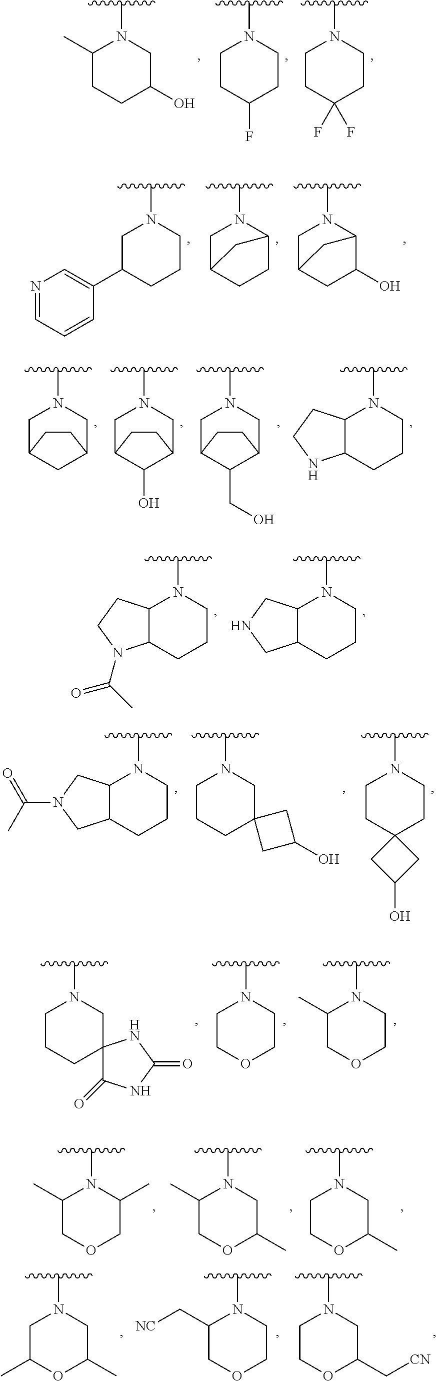

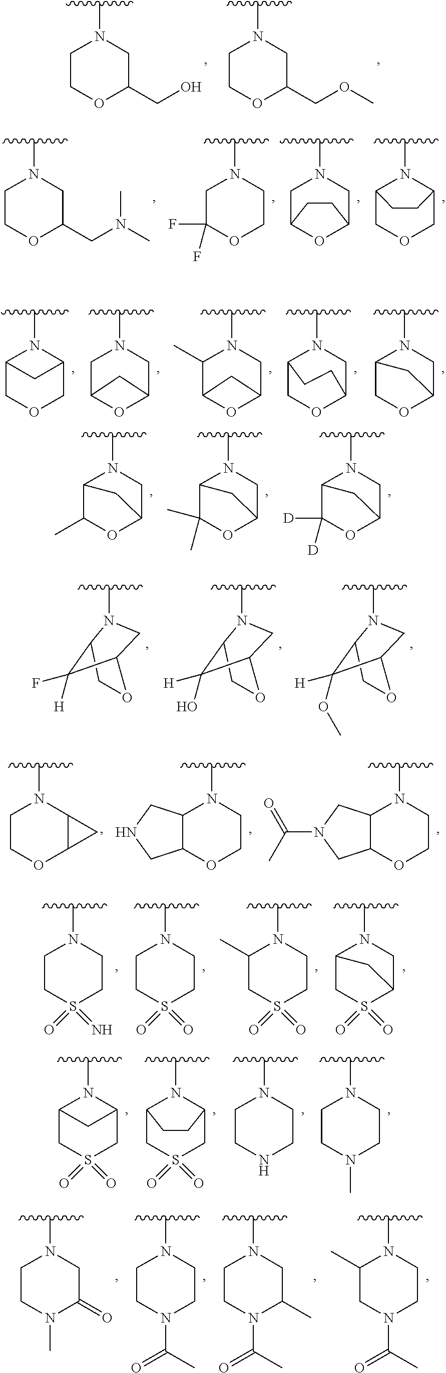

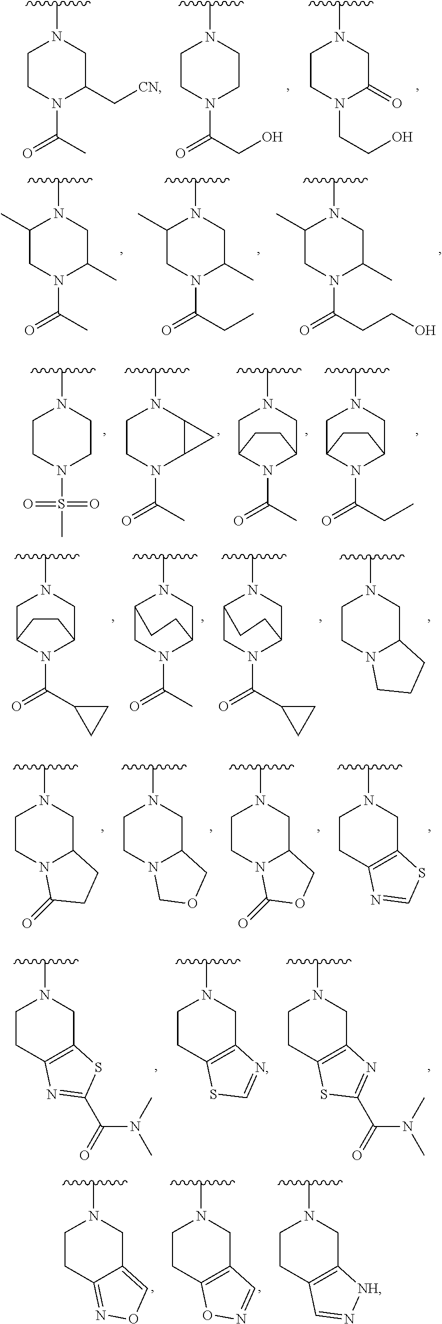

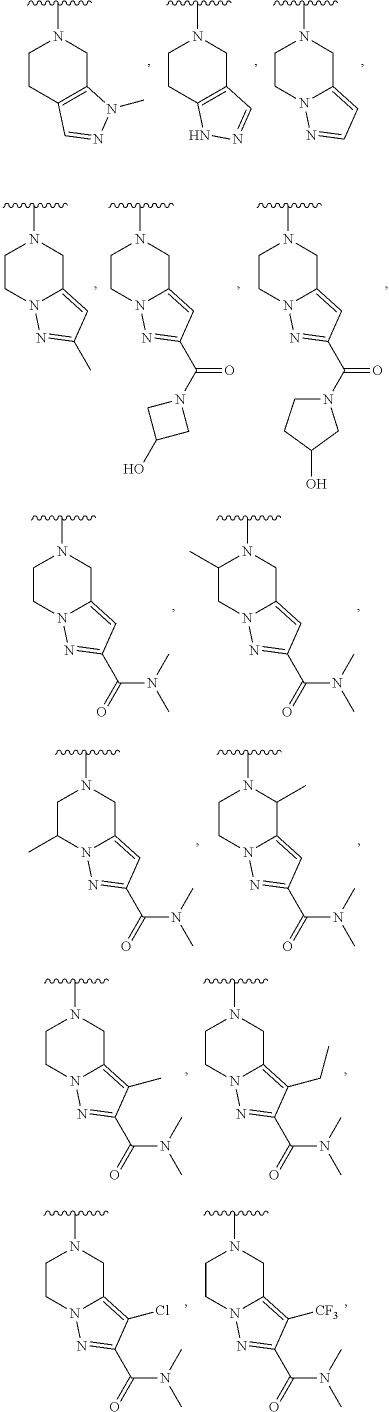

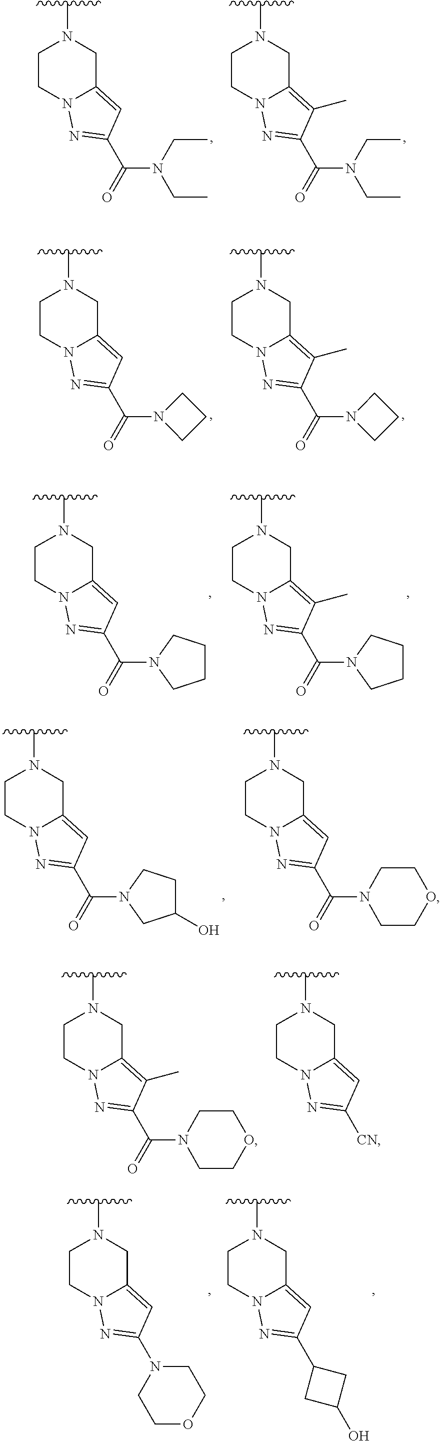

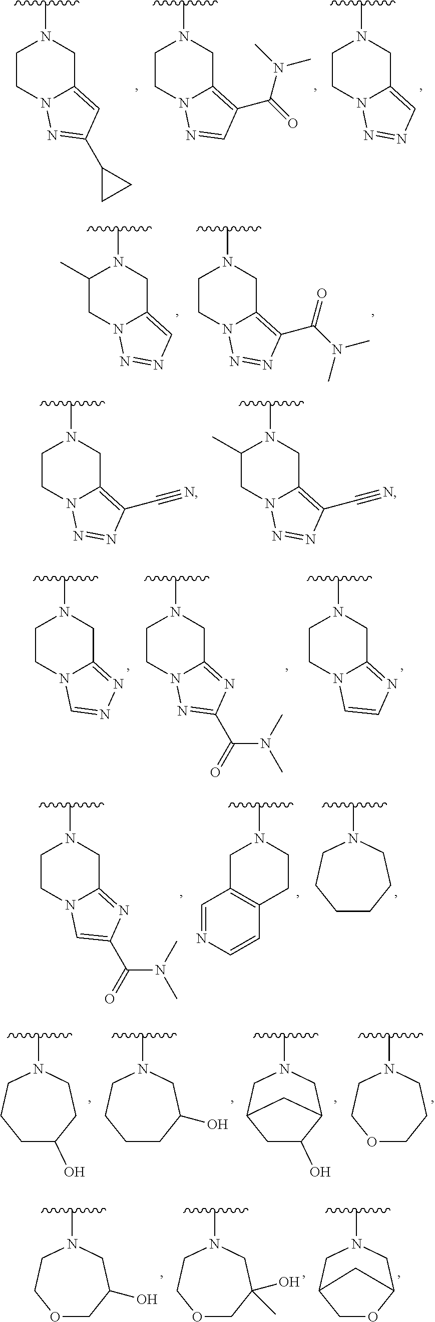

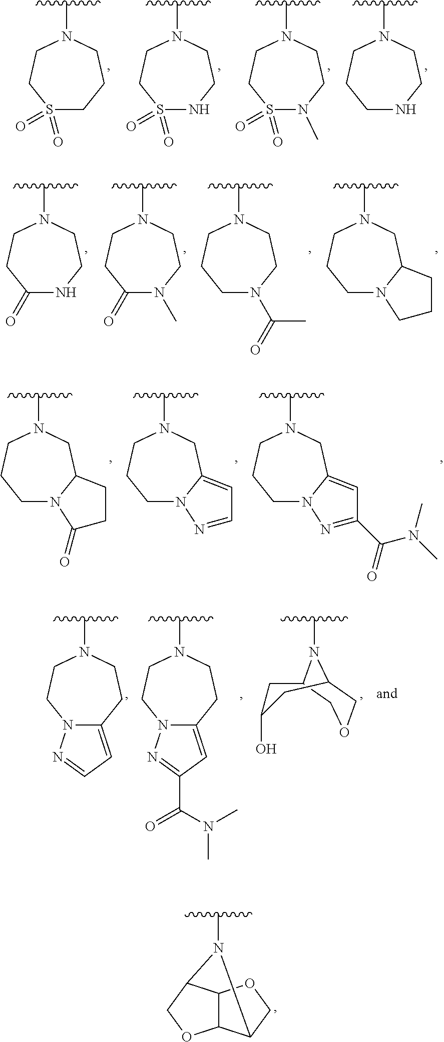

any of which is optionally further substituted with one or more R¹¹.

In some embodiments, the compound is a compound according to Formula IIIA:

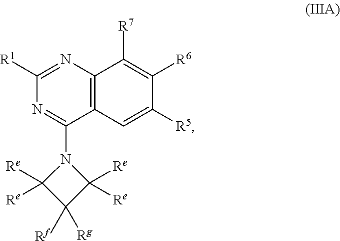

or a salt (e.g., pharmaceutically acceptable salt), ester, tautomer, prodrug, zwitterionic form, or stereoisomer(s) thereof, wherein:
R¹ is selected from —OR⁸ and

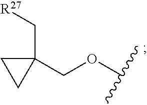

R¹ is selected from halogen and C₁₋₆alkyl, wherein any C₁₋₆alkyl is unsubstituted or substituted with one or more R¹¹;
R⁶ is a monocyclic or bicyclic heteroaryl, wherein the heteroaryl is substituted with one or more R¹⁵;
R¹ is selected from halogen;
R¹ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R² is independently selected from C₁₋₄ alkyl, C₂₋₆ alkenyl, and H, wherein any C₁₋₆alkyl or C₂₋₆ alkenyl is unsubstituted or substituted with one or more R¹¹;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹¹;
each R²⁸ is independently selected from —OH, —OC₁₋₆alkyl, —CN, —NH₂, —NHC₁alkyl, and halogen;
R²⁷ is a 3-6 membered heterocycle including one or more heteroatoms selected from N, O, and S, wherein the heterocycle is unsubstituted or substituted with one or more R¹¹;
each R²⁸ is independently selected from C₁₋₆alkyl and halogen;
R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹¹:
each R^{e} is independently selected from hydrogen, deuterium, —OR¹², ═O, ═N(R¹⁴), —C(O)(C₁₋₆alkylene)CN, —C(O(C₁₋₆alkylene)OR¹⁴, —C(O)(C₁₋₆alkyl), —C(O)N(R¹²)₂, —S(O)₂(C₁₋₆ alkyl), —N(R¹²)C(O(C₁₋₆alkyl), —N(R¹²)C(O)(C₁₋₆alkylene)OR¹⁴, halogen, and C₁₋₆alkyl, wherein any C₁₋₆alkyl is unsubstituted or substituted with one or more R²⁰; and
R^{f} and R^{g} are each independently selected from hydrogen, deuterium, —OR¹², ═O, —C(O)(C₁₋₆alkylene)CN, —C(O)(C₁₋₆alkylene)OR¹⁴, —C(O)(C₁₋₆-alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰, or
R^{f} and R^{g} join together to form a 3-6 membered carbocycle or heterocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰.

In some embodiments, the present disclosure provides a compound of Formula IIIA or a salt (e.g., a pharmaceutically acceptable salt) thereof.

In some embodiments, for a compound according to Formula IIIA, each R^{e}, R^{f}, and R^{g} is independently selected from hydrogen, —OR¹², ═O, —C(O)(C₁₋₆alkylene)CN, —C(O)(C₁₋₆akylene)OR¹⁴, —C(O)(C₁₋₆alkyl), —S(O)₂(C₁₋₆akyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰. In some embodiments, each R^{e}, R^{f}, and R^{g} is independently selected from hydrogen, —OR², ═O, —C(O)(C₁₋₆alkylene)CN, —C(O)(C₁₋₆alkylene)OR¹⁴, —C(O)(C₁₋₆akyl), —S(O)₂(C₁₋₆alkyl), —N(R¹⁴)C(O)(C₁₋₆akyl), —N(R¹⁴)C(O)(C₁₋₆ alkylene)OR¹⁴, halogen, and C₁₋₆alkyl that is unsubstituted or substituted with one or more R²⁰. It will be appreciated that when R^{e}, R^{f}, or R^{g} is ═O, the other R^{e}, R^{f}, or R^{g} on the same carbon atom is absent, such that the carbon has proper valency. In some embodiments, each R^{e} is independently hydrogen. In some embodiments, each R^{e}, R^{f}, and R^{g} is independently hydrogen.

In some embodiments, for a compound according to Formula IIIA, R^{f} and R^{g} join together to form a 3-6 membered carbocycle or heterocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆ alkyl), —N(R¹⁴)C(O)C₁₋₆alkyl), —N(R¹⁴)C(O)C₁₋₆alkylene)OR¹⁴, halogen, and C₁₋₆alkyl, wherein any C₁₋₆ alkyl is unsubstituted or substituted with one or more R²⁰. In some embodiments, R^{f} and R^{g} join together to form a 3-6 membered carbocycle or heterocycle that is unsubstituted. In some embodiments, R^{f} and R^{g} join together to form a 3-6 membered carbocycle or heterocycle that is substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹²)C(O)(C₁₋₆alkylene)OR¹⁴, halogen, and C₁₋₆alkyl, wherein any C₁₋₆alkyl is unsubstituted or substituted with one or more R²⁰. In some embodiments, R^{f} and R^{g} join together to form a 3-6 membered carbocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰. In some embodiments, R^{f} and R^{g} join together to form a cyclopropane that is unsubstituted or substituted with one or more substituents selected from —OR¹², ═O, —C(O)(C₁₋₆alkylene)CN, —C(O)(C₁₋₆alkylene)OR¹³, —C(O)(C₁₋₆akyl), —S(O)₂(C₁₋₆alkyl), —N(R¹²)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰. In some embodiments, R^{f} and R^{g} join together to form a cyclobutane that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆ alkyl), —S(O)₂(C₁₋₆alkyl), —N(R¹²)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R¹¹. In some embodiments, R^{f} and R^{g} join together to form a 3-6 membered heterocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹²)C(O)(C₁₋₆alkylene)OR¹⁴, halogen, and C₁₋₆alkyl, wherein any C₁₋₆alkyl is unsubstituted or substituted with one or more R²⁰. In some embodiments, R^{f} and R^{g} join together to form an oxetane that is unsubstituted or substituted with one or more substituents selected from —OR², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰.

In some embodiments, for a compound according to Formula IIIA,

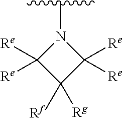

is a structure selected from:

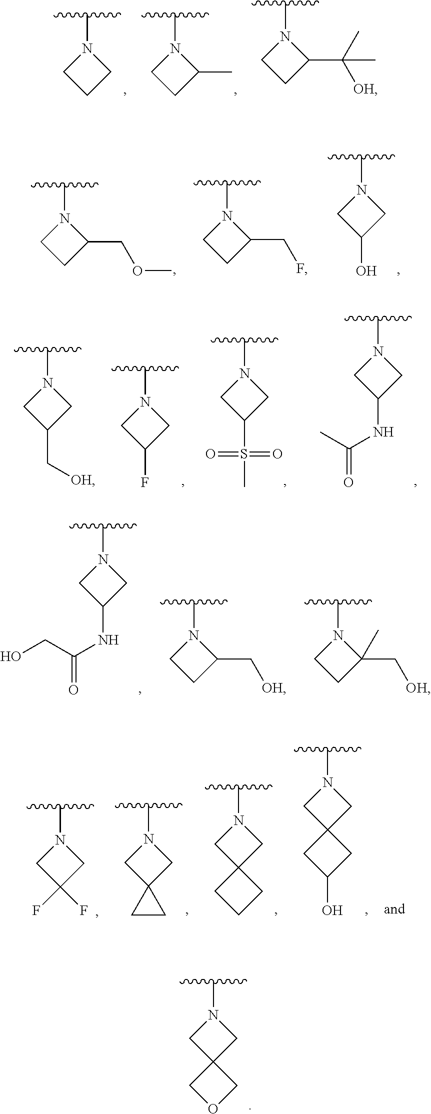

In some embodiments, the compound is a compound according to Formula IIIA1:

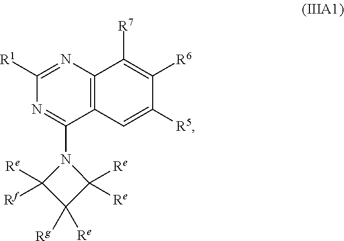

or a salt (e.g., pharmaceutically acceptable salt), ester, tautomer, prodrug, zwitterionic form, or stereoisomer(s) thereof, wherein:
R¹ is selected from —OR⁸ and

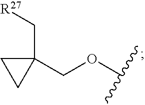

R⁵ is selected from halogen and C₁₋₆alkyl, wherein any C₁₋₆alkyl is unsubstituted or substituted with one or more R¹¹;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b} and wherein an alkyl moiety ofany alkylheterocycle is selected from C₁₋₆alkyl;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹², —CN, —N(R¹²)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₄alkyl, wherein any C₁₋₆alkyl is unsubstituted or substituted with one or more R¹¹;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²¹;
each R²⁸ is independently selected from C₁₋₆alkyl and halogen;
R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³;
each R^{e} is independently selected from hydrogen, deuterium, —OR¹², ═O, ═N(R¹²), —C(O)(C₁₋₆alkylene)CN, —C(O)(C₁₋₆alkylene)OR¹⁴, —C(O)(C₁₋₆alkyl), —C(O)(3-6 membered carbocycle), —C(O)N(R¹⁴)₂, —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰; and
R^{f} and R^{g} join together to form a 3-6 membered carbocycle or heterocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —C(O)O(C₁₋₆alkyl), —C(O)N(R¹⁴)₂, —C(O)(3-6 membered carbocycle or heterocycle), —S(O)₂(C₁₋₆alkyl), —N(R¹²)C(O)(C₁₋₆alkyl), —N(R¹²)C(O)(C₁₋₆alkylene)OR¹⁴, halogen, and C₁₋₆alkyl, wherein any C₁₋₆alkyl, carbocycle, or heterocycle is unsubstituted or substituted with one or more R²⁰.

In some embodiments, the present disclosure provides a compound of Formula IIIA1 or a salt (e.g., a pharmaceutically acceptable salt) thereof.

In some embodiments, for a compound according to Formula IIIA1, each R²⁰ is independently selected from hydrogen, —OR¹², ═O, —C(O)C₁₋₆alkylene)CN, —C(O)(C₁₋₆alkylene)OR¹⁴, —C(O)(C₁₋₆alkyl), —S(O)₂(C₁₋₆alkyl), —N(R¹²)C(O)(C₁₋₆alkyl), —N(R¹²)C(O)(C₁₋₆alkylene)OR¹⁴, halogen, and C₁₋₆alkyl, wherein any C₁₋₆alkyl is unsubstituted or substituted with one or more R²⁰. In some embodiments, for a compound according to Formula IIIA1, each R^{c} is independently selected from hydrogen, —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹²)C(O)C₁₋₆alkylene)OR¹⁴, halogen, and C₁₋₆alkyl that is unsubstituted or substituted with one or more R²⁰. It will be appreciated that when R^{e} is attached to a carbon that is also attached to R^{f} or R^{g}, R^{e} is not ═O or ═N(R¹⁴), and when R^{e} is ═O or ═N(R¹⁴), the other R^{e} on the same carbon atom is absent, such that the carbon has proper valency. In some embodiments, each R is independently hydrogen.

In some embodiments, for a compound according to Formula IIA1, R^{f} and R^{g} join together to form a 3-6 membered carbocycle or heterocycle that is unsubstituted. In some embodiments, R^{f} and R^{g} join together to form a 3-6 membered carbocycle or heterocycle that is substituted with one or more substituents selected from —OR¹², ═O, —C(O)C₁₋₆alkylene)CN, —C(O)(C₁₋₆alkylene)OR¹⁴, —C(O)(C₁₋₆alkyl), —C(O)O(C₁₋₆ alkyl), —C(O)N(R¹⁴)₂, —C(O)(3-6 membered carbocycle or heterocycle), —S(O)₂(C₁₋₆alkyl), —N(R¹²)C(O)(C₁₋₆ alkyl), —N(R¹⁴)C(O)(C₁₋₆alkylene)OR¹⁴, halogen, and C₁₋₆alkyl, wherein any C₁₋₆alkyl, carbocycle, or heterocycle is unsubstituted or substituted with one or more R²⁰. In some embodiments, R^{f} and R^{g} join together to form a 3-6 membered carbocycle or heterocycle that is substituted with one or more substituents selected from —OR¹², ═O, —C(O)(C₁₋₆alkylene)CN, —C(O)(C₁₋₆alkylene)OR¹⁴, —C(O)(C₁₋₆alkyl), —C(O)(3-6 membered carbocycle or heterocycle), —S(O)₂(C₁₋₆alkyl), —N(R¹⁴)C(O)(C₁₋₆alkyl), —N(R¹²)C(O)(C₁₋₆ alkylene)OR¹⁰, halogen, and C₁₋₆alkyl, wherein any C₁₋₆alkyl, carbocycle, or heterocycle is unsubstituted or substituted with one or more R¹¹. In some embodiments, R^{f} and R^{g} join together to form a 3-6 membered carbocycle or heterocycle that is substituted with one or more substituents selected from —OR¹², ═O, —C(O)(C₁₋₆alkylene)CN, —C(O)(C₁₋₆alkylene)OR¹⁴, —C(O)(C₁₋₆alkyl), —C(O)(3-6 membered carbocycle or heterocycle), —S(O)₂(C₁₋₆alkyl), —N(R¹²)C(O)(C₁₋₆alkyl), —N(R¹²)C(O)(C₁₋₆alkylene)OR¹⁴, halogen, and C₁₋₆alkyl that is unsubstituted or substituted with one or more R²⁰, wherein any carbocycle or heterocycle is unsubstituted or substituted with one or more R²⁰. In some embodiments, R^{f} and R^{g} join together to form pyrrolidine that is substituted with one or more substituents selected from —OR¹², —O, —C(O)(C₁₋₆alkylene)CN, —C(O)(C₁₋₆alkylene)OR¹⁴, —C(O)(C₁₋₆alkyl), —C(O)(3-6 membered carbocycle or heterocycle), —S(O)₂(C₁₋₆alkyl), —N(R¹²)C(O)(C₁₋₆alkyl), —N(R¹⁴)C(O)(C₁₋₆alkylene)OR¹⁴, halogen, and C₁₋₆ alkyl, wherein any C₁₋₆alkyl, carbocycle, or heterocycle is unsubstituted or substituted with one or more R²⁰. In some embodiments, R^{f} and R^{g} join together to form pyrrolidine that is substituted with one or more substituents selected from —OR¹⁰, ═O, —C(O)(C₁₋₆alkylene)CN, —C(O)(C₁₋₆alkylene)OR¹⁴, —C(O)C₁₋₆alkyl), —C(O)O(C₁₋₆akyl), —C(O)N(R¹⁴), —C(O)(3-6 membered carbocycle or heterocycle), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl, carbocycle, or heterocycle is unsubstituted or substituted with one or more R²⁰. In some embodiments, when the ring formed by joining R^{f} and R^{g} together is substituted with —C(O)N(R¹⁴)₂, at least one R¹⁴ is C₁₋₆alkyl.

In some embodiments, for a compound according to Formula IIIA1,

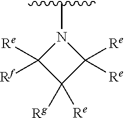

is a structure selected from:

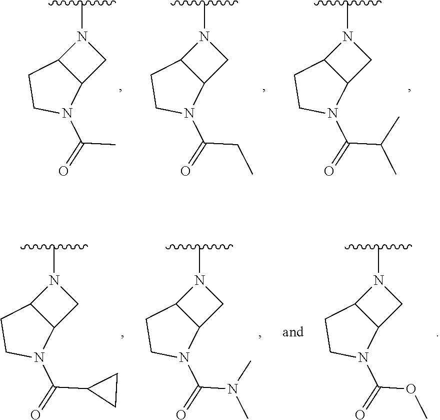

In some embodiments, the compound is a compound according to Formula IIIB:

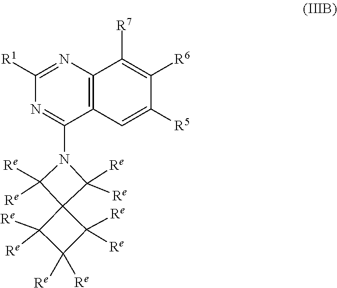

or a salt (e.g., pharmaceutically acceptable salt), ester, tautomer, prodrug, zwitterionic form, or stereoisomer(s) thereof, wherein:
R¹ is selected from —OR⁸ and

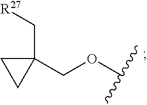

R⁵ is selected from halogen and C₁₋₆alkyl, wherein any C₁₋₆alkyl is unsubstituted or substituted with one or more R¹¹;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R or R, and wherein an alkyl moiety of any alkylheterocycle is selected from C₁₋₆alkyl;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹¹;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹³;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R¹¹,
each R²⁸ is independently selected from C₁₋₆alkyl and halogen;
R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹¹; and
each R^{e} is independently selected from hydrogen, deuterium, —OR², ═O, ═N(R¹²), —C(O)(C₁₋₆alkylene)CN, —C(O)(C₁₋₆alkylene)OR¹⁴, —C(O)(C₁₋₆alkyl), —C(O)N(R¹⁴)₂, —S(O)₂(C₁₋₆ alkyl), —N(R¹⁴)C(O)(C₁₋₆alkyl), —N(R¹⁴)C(O)(C₁₋₆alkylene)OR¹⁰, halogen, and C₁₋₆alkyl, wherein any C₁₋₆alkyl is unsubstituted or substituted with one or more R²⁰.

In some embodiments, the present disclosure provides a compound of Formula IIIB or a salt (e.g., a pharmaceutically acceptable salt) thereof.

In some embodiments, for a compound according to Formula IIIB, each R^{e} is hydrogen. In some embodiments, at least one R^{e} is —OR¹². In some embodiments, each R^{e} is independently selected from hydrogen, —OR¹², ═O, —C(O)C₁₋₆alkylene)CN, —C(O)C₁₋₆alkylene)OR¹⁴, —C(O)(C₁₋₆alkyl), —S(O)₂(C₁₋₆alkyl), —N(R¹²)C(O(C₁₋₆alkyl), —N(R¹²)C(O(C₁₋₆alkylene)OR¹¹, halogen, and C₁₋₆alkyl that is unsubstituted or substituted with one or more R²⁰. It will be appreciated that when R^{e} is —O, the other R^{e} on the same carbon atom is absent, such that the carbon has proper valency.

In some embodiments, the compound is a compound according to Formula IIIC:

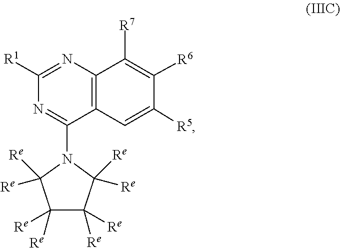

or a salt (e.g., pharmaceutically acceptable salt), ester, tautomer, prodrug, zwitterionic form, or stereoisomer(s) thereof wherein:
R¹ is selected from —OR⁸ and

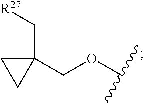

R⁵ is selected from halogen and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹¹;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹¹;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R¹¹;
each R²⁸ is independently selected from C₁₋₆alkyl and halogen;
R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹¹; and
each R^{e} is independently selected from hydrogen, deuterium, —OR¹², ═O, ═N(R¹⁴), —C(O)(C₁₋₆alkylene)CN, —C(O)(C₁₋₆alkylene)OR¹⁴, —C(O)(C₁₋₆alkyl), —C(O)N(R¹⁴)₂, —S(O)₂(C₁₋₆ alkyl), —N(R¹²)C(O)(C₁₋₆alkyl), —N(R¹⁴)C(O)(C₁₋₆alkylene)OR¹⁴, halogen, a 5-6 membered heteroaryl, and C₁₋₆alkyl, wherein any C₁₋₆alkyl or heteroaryl is unsubstituted or substituted with one or more R²⁰.

In some embodiments, the present disclosure provides a compound of Formula IIIC or a salt (e.g., a pharmaceutically acceptable salt) thereof.

In some embodiments, for a compound according to Formula IIIC,

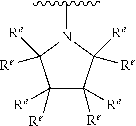

is a structure selected from:

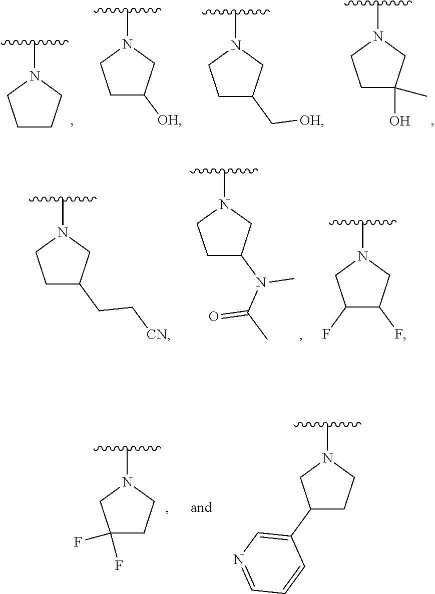

In some embodiments, the compound is a compound according to Formula IIIC1:

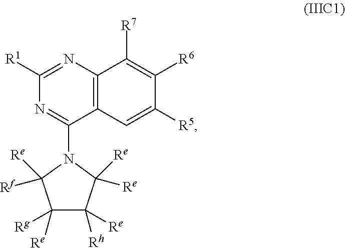

or a salt (e.g., pharmaceutically acceptable salt), ester, tautomer, prodrug, zwitterionic form, or stereoisomer(s) thereof, wherein:
R¹ is selected from —OR⁸ and

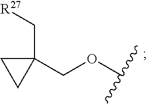

R⁵ is selected from halogen and C₁₋₆alkyl, wherein any C₁₋₆alkyl is unsubstituted or substituted with one or more R¹¹;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹² is independently selected from C₁₋₆ alkyl, C₂₋₄ alkenyl, and H, wherein any C₁₋₆alkyl or C₂₋₆ alkenyl is unsubstituted or substituted with one or more R¹¹,
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹¹;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen;
R^{a} and R^{b} are each independently selected from deuterium, halogen, C₁₋₄ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹¹;
each R^{e} is independently selected from hydrogen, deuterium, —OR², ═O, ═N(R¹²), —C(O)(C₁₋₆alkylene)CN, —C(O)(C₁₋₆alkylene)OR¹⁴, —C(O)(C₁₋₆alkyl), —C(O)N(R¹⁴)₂, —S(O)₂(C₁₋₆ alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰; and
R^{f}, R^{g}, and R^{h} are each independently selected from hydrogen, deuterium, —OR¹², —O, —C(O)(C₁₋₆ alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R¹⁰, optionally wherein
(i) R^{f} and R^{g} join together to form a 3-6 membered carbocycle or heterocycle or a 5-6 membered heteroaryl that is unsubstituted or substituted with one or more substituents selected from —OR¹², —O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹²)C(O)C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰; or
(ii) R^{g} and R^{h} join together to form a 3-6 membered carbocycle or heterocycle or a 5-6 membered heteroaryl that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰.

In some embodiments, the present disclosure provides a compound of Formula IIIC1 or a salt (e.g., a pharmaceutically acceptable salt) thereof.

In some embodiments, for a compound according to Formula IIIC or IIIC1, each R^{e} is hydrogen. In some embodiments, at least one R^{e} is selected from —OR¹², halogen, and C₁₋₆alkyl that is unsubstituted or substituted with one or more R²⁰ (e.g., C₁₋₆ alkyl substituted with —OH). In some embodiments, each R⁶ is independently selected from hydrogen, —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl that is unsubstituted or substituted with one or more R²⁰. It will be appreciated that when R^{e} is ═O, the other R^{e}, R^{f}, R^{g}, or R^{h} on the same carbon atom is absent, such that the carbon has proper valency.

In some embodiments, for a compound according to Formula IIIC1, (i) R^{f} and R^{g} or (ii) R^{g} and R^{h} join together to form a 3-6 membered carbocycle or heterocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰. In some embodiments, R^{f} and R^{g} join together to form a 3-6 membered carbocycle or heterocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹²)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R. In some embodiments, R^{g} and R^{h} join together to form a 3-6 membered carbocycle or heterocycle that is unsubstituted or substituted with one or more substituents selected from —OR¹², ═O, —C(O)(C₁₋₆alkylene)CN, —C(O)(C₁₋₆ alkylene)OR¹, —C(O)(C₁₋₆alkyl), —S(O)₂(C₁₋₆alkyl), —N(R¹⁰)C(O)(C₁₋₆alkyl), —N(R¹²)C(O)(C₁₋₆alkylene)OR¹⁴, halogen, and C₁₋₆alkyl, wherein any C₁₋₆alkyl is unsubstituted or substituted with one or more R²⁰. In some embodiments, (i) R^{f} and R^{g} or (ii) R^{g} and R^{h} join together to form a 3-6 membered carbocycle or heterocycle that is unsubstituted. In some embodiments, (i) R^{f} and R^{g} or (ii) R^{g} and R^{h} join together to form a 3-6 membered carbocycle or heterocycle that is substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰. In some embodiments, (i) R^{f} and R^{g} or (ii) R⁹ and R join together to form a 3-6 membered heterocycle that is unsubstituted or substituted with one or more substituents selected from —OR¹², —O, —C(O)(C₁₋₆alkylene)CN, —C(O)(C₁₋₆alkylene)OR¹⁴, —C(O)(C₁₋₆ alkyl), —S(O)₂(C₁₋₆alkyl), —N(R¹²)C(O)C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰. In some embodiments, (i) R^{f} and R^{g} or (ii) R^{g} and R^{h} join together to form an tetrahydrofuran that is unsubstituted or substituted with one or more substituents selected from —OR¹², ═O, —C(O)(C₁₋₆alkylene)CN, —C(O)(C₁₋₆alkylene)OR¹⁴, —C(O)C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R. In some embodiments, (i) R^{f} and R^{g} or (ii) R^{g} and R^{h} join together to form a pyrrolidine that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰. In some embodiments, for a compound according to Formula IIIC1, (i) R^{f} and R^{g} or (ii) R^{f} and R^{h} join together to form a 5-6 membered heteroaryl that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)C₁₋₆alkyl), —N(R¹⁴)C(O)C₁₋₆alkylene)OR¹⁴, halogen, and C₁₋₆alkyl, wherein any C₁₋₆alkyl is unsubstituted or substituted with one or more R²⁰. In some embodiments, R^{f} and R^{g} join together to form a 5-6 membered heteroaryl that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁰, halogen, and C₁₋₆alkyl, wherein any C₁₋₆alkyl is unsubstituted or substituted with one or more R²⁰. In some embodiments, R^{g} and R^{h} join together to form a 5-6 membered heteroaryl that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹²)C(O)(C₁₋₆alkyl), —N(R¹⁴)C(O)(C₁₋₆alkylene)OR¹⁰, halogen, and C₁₋₆alkyl, wherein any C₁₋₆alkyl is unsubstituted or substituted with one or more R²⁰. In some embodiments, (i) R^{f} and R^{g} or (ii) R^{g} and R^{h} join together to form a 5-6 membered heteroaryl that is unsubstituted (e.g., a pyridine). In some embodiments, (i) R^{f} and R^{g} or (ii) R^{g} and R^{h} join together to form a 5-6 membered heteroaryl that is substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹²)C(O)(C₁₋₆ alkylene)OR¹⁴, halogen, and C₁₋₆alkyl, wherein any C₁₋₆alkyl is unsubstituted or substituted with one or more R²⁰. In some embodiments, the ring formed when (i) R^{f} and R^{g} or (ii) R^{g} and R^{h} join together does not comprise an —NH— moiety. It will be appreciated that when an R^{e}, R^{f}, R^{g}, or R^{h} is ═O, another optional substitutent that would otherwise be on the same carbon atom is absent, such that the carbon has proper valency.

In some embodiments, for a compound according to Formula IIIC1,

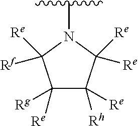

is a structure selected from:

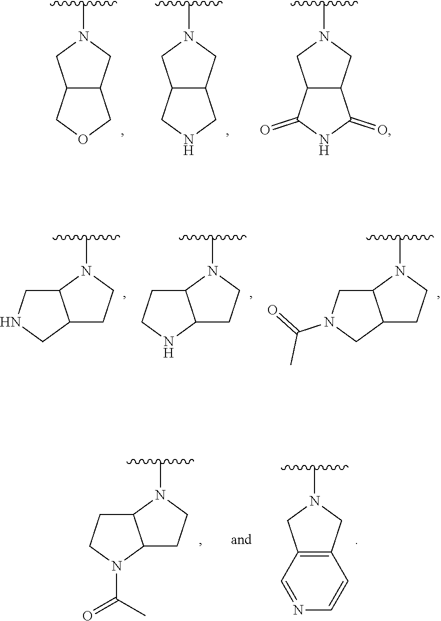

In some embodiments, the compound is a compound according to Formula IIID:

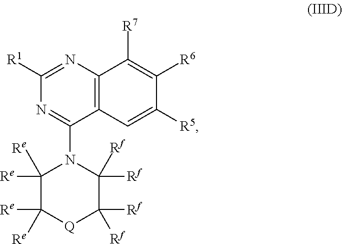

or a salt (e.g., pharmaceutically acceptable salt), ester, tautomer, prodrug, zwitterionic form, or stereoisomer(s) thereof, wherein:
R¹ is selected from —OR⁸ and

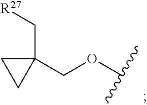

R⁵ is selected from halogen and C₁₋₆alkyl, wherein any C₁₋₆alkyl is unsubstituted or substituted with one or more R¹¹;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹¹ is independently selected from deuterium, —OR², ═O, ═N(R¹²), —C(O)(C₁₋₆alkylene)CN, —C(O)(C₁₋₆alkylene)OR¹⁴, —C(O)(C₁₋₆alkyl), —C(O)O(C₁₋₆alkyl), —C(O)N(R¹⁴)₂, —C(O)(3-6 membered carbocycle or heterocycle), —S(O)₂(C₁₋₆alkyl), —N(R¹²)C(O)(C₁₋₆alkyl), —N(R¹²)C(O)(C₁₋₆alkylene)OR¹⁴, halogen, —CN, a 3-6 membered carbocycle or heterocycle, a 5-6 membered heteroaryl, and C₁₋₆alkyl, wherein any C₁₋₆alkyl, carbocycle, heterocycle, or heteroaryl is unsubstituted or substituted with one or more R²⁰;
each R¹² is independently selected from C₁₋₆alkyl, C₂₋₆ alkenyl, and H, wherein any C₁₋₆alkyl or C₂₋₆alkenyl is unsubstituted or substituted with one or more R¹¹;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹¹;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R¹¹;
each R²⁸ is independently selected from C₁₋₆alkyl and halogen;
R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³;
Q is selected from CR^{h}R^{j}, NR², O, S, and SO₂:
each R^{e} and R^{f} is independently selected from R¹¹ and hydrogen, wherein:
   (i) an R^{e} and an R^{f} can optionally join together to form a 4-6 membered ring;
   (ii) a first R^{f} and a second R^{f} connected to adjacent atoms can optionally join together to form a 3-5 membered ring;
   (iii) a first R^{e} and a second R^{e} connected to adjacent atoms can optionally join together to form a 3-5 membered ring; or
   (iv) a first R^{f} and a second R^{f} connected to the same atom can optionally join together to form a 3-5 membered ring,
   wherein any ring formed by one or more R^{f} and/or one or more R^{f} is unsubstituted or substituted with one or more R¹¹;
R^{g}, when present, is R¹¹, and
R^{h} and R^{j}, when present, are each independently selected from R¹¹ and hydrogen, or can optionally join together to form a 3-4 membered carbocycle or heterocycle that is unsubstituted or substituted with one or more substituents selected from —OR¹², ═O, ═N(R¹⁴), —C(O)(C₁₋₆alkylene)CN, —C(O)(C₁₋₆alkylene)OR¹⁴, —C(O)(C₁₋₆alkyl), —C(O)N(R¹²)₂, —S(O)₂(C₄alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰, or a R^{h} and a R^{e} or a R^{h} and a R^{f} optionally join together to form a 3-6 membered ring that is unsubstituted or substituted with one or more R¹¹.

In some embodiments, the present disclosure provides a compound of Formula IIID or a salt (e.g., a pharmaceutically acceptable salt) thereof.

In some embodiments, for a compound according to Formula IIID, Q is NR^{g}. In some embodiments, R⁹ is selected from —C(O)(C₁₋₆alkylene)CN, —C(O)(C₁₋₆alkylene)OR¹⁴, —C(O)(C₁₋₆alkyl), —S(O)₂(C₁₋₆alkyl), and C₁₋₆alkyl, wherein any C₁₋₆alkyl is unsubstituted or substituted with one or more R²⁰. In some embodiments, R^{g} is selected from —C(O)(C₁₋₆alkylene)CN, —C(O)(C₁₋₆alkylene)OR¹⁴, —C(O)(C₁₋₆ alkyl), —S(O)₂(C₁₋₆alkyl), and C₁₋₆alkyl that is unsubstituted or substituted with one or more R²⁰.

In some embodiments, for a compound according to Formula IIID, Q is O, S, or SO₂. In some embodiments, Q is O. In some embodiments, Q is S. In some embodiments, Q is SO₂.

In some embodiments, for a compound according to Formula IIID, Q is CR^{h}Rⁱ. In some embodiments, Q is CR^{k}R^{j} and an R^{e} and an R^{f} join together to form a 4-6 membered ring. In some embodiments, when Q is CR^{h}Rⁱ and an R^{e} and an R^{f} join together to form a 4-6 membered ring. R^{h} and R^{j} do not join together to form a 3-4 membered carbocycle or heterocycle. In some embodiments, Q is CR^{h}R^{j} and R^{h} and R^{j} join together to form a 3-4 membered carbocycle or heterocycle. In some embodiments, when Q is CR^{k}R^{j} and R^{h} and R^{j} join together to form a 3-4 membered carbocycle or heterocycle, no combination of an R^{e} and an R^{f} join together to form a 4-6 membered ring. In some embodiments, when Q is CR^{h}R^{j} and R^{h} and R^{j} join together to form a 3-4 membered carbocycle or heterocycle, no combination of one or more R^{e}s and/or one or more R^{f}s join together to form a ring. In some embodiments, Q is CR^{h}Rⁱ and R^{h} and R^{j} are each hydrogen. In some embodiments, Q is CR^{h}R^{j}, R^{h} is hydrogen, and R^{j} is selected from deuterium, —OR¹⁴, ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹²)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰. In some embodiments, Q is CR^{h}R^{j}, R^{j} is hydrogen, and R^{j} is selected from —OR¹², —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹²)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R². In some embodiments, Q is CR^{h}R^{j}, R^{h} is hydrogen, and R¹ is selected from —OR¹², —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl that is unsubstituted or substituted with one or more R²⁰. In some embodiments, Q is CR^{h}R^{j}, R^{j} is hydrogen, and a R^{h} and a R^{e} or a R^{h} and a R^{f} optionally join together to form a 3-6 membered ring (e.g., a pyridine) that is unsubstituted or substituted with one or more R¹¹.

In some embodiments, for a compound according to Formula IIID, R^{h} and R^{j} are independently selected from R¹¹ and hydrogen. In some embodiments, each R¹¹ is independently selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹²)C(O)C₁₋₆alkyl), —N(R¹²)C(O)(C₁₋₆alkylene)OR¹⁴, halogen, and C₁₋₆alkyl that is unsubstituted or substituted with one or more R²⁰. In some embodiments, at least one of R^{h} and R^{j} is —OR¹¹ or C₁₋₆alkyl that is unsubstituted or substituted with one or more R(e.g., C₁₋₆ alkyl that is unsubstituted or substituted with —OH or —CN). In some embodiments, R^{h} and R^{j} are hydrogen. It will be appreciated that when R^{h} or R^{j} is ═O, the R^{h} or R^{j} on the same carbon atom is absent, such that the carbon has proper valency.

In some embodiments, for a compound according to Formula IIID, R^{h} and R^{j} join together to form a 3-4 membered carbocycle or heterocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹²)C(O)(C₁₋₆alkylene)OR¹⁴, halogen, and C₁₋₆alkyl that is unsubstituted or substituted with one or more R²⁰. In some embodiments, at least one of R^{h} and R^{j} is —OR¹² or C₁₋₆alkyl that is unsubstituted or substituted with one or more R²⁰ (e.g., C₁₋₆ alkyl that is unsubstituted or substituted with —OH or —CN). In some embodiments, both R^{h} and R^{j} are hydrogen. It will be appreciated that when R^{h} or R^{j} is ═O, the other R^{h} or R^{j} on the same carbon atom is absent, such that the carbon has proper valency.

In some embodiments, for a compound according to Formula IIID, each R^{e} and R^{f} is independently selected from R¹¹ and hydrogen. In some embodiments, each R¹⁵ is independently selected from deuterium, —OR¹², —O, —N(R¹²), —C(O)(C₁₋₆alkylene)CN, —C(O)(C₁₋₆alkylene)OR¹⁴, —C(O)(C₁₋₆alkyl), —C(O)N(R¹²)₂, —S(O)₂(C₁₋₆alkyl), —N(R¹²)C(O)(C₁₋₆alkyl), —N(R¹²)C(O)(C₁₋₆alkylene)OR¹⁴, halogen, and C₁₋₆alkyl that is unsubstituted or substituted with one or more R²⁰. In some embodiments, at least one of R^{e} and R^{f} is —OR¹² or C₁₋₆alkyl that is unsubstituted or substituted with one or more R²⁰ (e.g., C₁₋₆ alkyl that is unsubstituted or substituted with —OH or —CN). In some embodiments, each R^{e} and R^{f} is independently selected from hydrogen and R¹¹, wherein each R¹¹ is independently selected from —OR¹², halogen, a 5-6 membered heteroaryl, and C₁₋₆alkyl that is unsubstituted or substituted with one or more R²⁰. In some embodiments, each R²⁰ is hydrogen. In some embodiments, each R^{f} is hydrogen. In some embodiments, each R^{e} and R^{f} is hydrogen. It will be appreciated that when R^{e} or R^{f} is ═O, the other R^{e} or R^{f} on the same carbon atom is absent, such that the carbon has proper valency.

In some embodiments, the compound is a compound according to Formula IIIE:

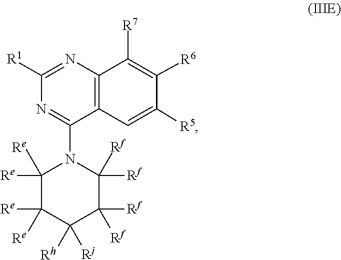

or a salt (e.g., pharmaceutically acceptable salt), ester, tautomer, prodrug, zwitterionic form, or stereoisomer(s) thereof, wherein:
R¹ is selected from —OR⁸ and

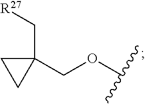

R⁵ is selected from halogen and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹¹ is independently selected from deuterium, —OR¹⁴, ═O, ═N(R¹⁴), —C(O)(C₁₋₆alkylene)CN, —C(O)(C₁₋₆alkylene)OR¹⁴, —C(O)(C₁₋₆alkyl), —C(O)N(R¹⁴)₂, —S(O)₂(C₁₋₆alkyl), —N(R¹²)C(O)(C₁₋₆alkyl), —N(R¹²)C(O(C₁₋₆alkylene)OR¹⁴, halogen, a 5-6 membered heteroaryl, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹¹;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹³;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen;
R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹¹:
each R^{e} and R^{f} is independently selected from R¹¹ and hydrogen, wherein:
   (i) an R^{e} and an R^{f} can optionally join together to form a 4-6 membered ring;
   (ii) a first R^{f} and a second R^{f} connected to adjacent atoms can optionally join together to form a 3-5 membered ring;
   (iii) a first R^{e} and a second R^{f} connected to adjacent atoms can optionally join together to form a 3-5 membered ring; or
   (iv) a first R^{f} and a second R^{f} connected to the same atom can optionally join together to form a 3-5 membered ring, wherein any ring formed by one or more R^{c} and/or one or more R^{f} is unsubstituted or substituted with one or more R¹¹; and
R^{h} and R^{j} are each independently selected from R¹¹ and hydrogen, or can optionally join together to form a 3-4 membered carbocycle or heterocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰, or a R^{h} and a R^{e} or a R^{h} and a R^{f} optionally join together to form a 3-6 membered ring that is unsubstituted or substituted with one or more R¹¹.

Tn some embodiments, the present disclosure provides a compound of Formula IIIE or a salt (e.g., a pharmaceutically acceptable salt) thereof.

In some embodiments, for a compound according to Formula IIIE, R^{h} and R^{j} are independently selected from R¹¹ and hydrogen. In some embodiments, each R¹¹ is independently selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹²)C(O)(C₁₋₆alkyl), —N(R¹⁴)C(O)(C₁₋₆alkylene)OR¹⁴, halogen, and C₁₋₆alkyl that is unsubstituted or substituted with one or more R²⁰. In some embodiments, at least one of R^{h} and R^{j} is —OR¹² or C₁₋₆alkyl that is unsubstituted or substituted with one or more R²⁰ (e.g., C₁₋₆ alkyl that is unsubstituted or substituted with —OH or —CN). In some embodiments, R^{h} is hydrogen and R^{j} is R¹¹. In some embodiments, R^{h} and R^{j} are hydrogen. It will be appreciated that when R^{h} or R^{j} is ═O, the R^{h} or R^{j} on the same carbon atom is absent, such that the carbon has proper valency. In some embodiments, R^{h} and R^{j} are independently selected from R¹¹ and hydrogen, and (i) an R^{e} and an R^{f} join together to form a 4-6 membered ring; (ii) a first R^{f} and a second R^{f} connected to adjacent atoms can optionally join together to form a 3-5 membered ring; (iii) a first R^{e} and a second R^{e} connected to adjacent atoms can optionally join together to form a 3-5 membered ring; or (iv) a first R^{f} and a second R^{f} connected to the same atom can optionally join together to form a 3-5 membered ring, wherein any ring formed by one or more R^{e} and/or one or more R^{f} is unsubstituted or substituted with one or more R¹¹.

In some embodiments, for a compound according to Formula IIIE, R^{h} and R^{j} join together to form a 3-4 membered carbocycle or heterocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆₆alkyl, wherein any C₁₋₆alkyl is unsubstituted or substituted with one or more R²⁰. In some embodiments, for a compound according to Formula IIIE, R^{h} and R^{j} join together to form a 3-4 membered carbocycle or heterocycle that is unsubstituted or substituted with one or more substituents selected from —OR¹², ═O, —C(O)(C₁₋₆ alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl that is unsubstituted or substituted with one or more R²⁰. In some embodiments, when R^{h} and R^{j} join together to form a 3-4 membered carbocycle or heterocycle, no combination of an R^{e} and an R^{f} join together to form a 4-6 membered ring. In some embodiments, when R^{h} and R^{j} join together to form a 3-4 membered carbocycle or heterocycle, no combination of one or more R^{e}s and/or one or more R^{f}s join together to form a ring. In some embodiments, R^{h} and R^{j} join together to form a 3-4 membered carbocycle or heterocycle that is unsubstituted. In some embodiments, R^{h} and R^{j} join together to form a 3-4 membered carbocycle or heterocycle that is substituted with one or more substituents selected from —OR¹², —O, —C(O)(C₁₋₆alkylene)CN, —C(O)(C₁₋₆alkylene)OR¹⁴, —C(O)C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰. In some embodiments, R^{h} and R^{j} join together to form a 3-4 membered carbocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹²)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰. In some embodiments, R^{h} and Rⁱ join together to form a cyclopropane that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹²)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁹. In some embodiments, R^{h} and R^{j} join together to form a cyclobutane that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹²)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R². In some embodiments, R^{h} and R^{j} join together to form a 3-4 membered heterocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹²)C(O)(C₁₋₄alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰. In some embodiments, R^{j} is hydrogen, and a R^{h} and a R^{e} or a R^{h} and a R^{f} optionally join together to form a 3-6 membered ring (e.g., a pyridine) that is unsubstituted or substituted with one or more R¹¹.

In some embodiments for a compound according to Formula IIIE, each R^{e} and R^{f} is independently selected from R¹¹ and hydrogen. In some embodiments, each R¹¹ is independently selected from deuterium, —OR¹², ═O, ═N(R¹⁴), —C(O)(C₁₋₆alkylene)CN, —C(O)(C₁₋₆alkylene)OR¹⁴, —C(O)C₁₋₆alkyl), —C(O)N(R¹²)₂, —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl that is unsubstituted or substituted with one or more R²⁰. In some embodiments, at least one of R^{e} and R^{f} is —OR¹² or C₁₋₆alkyl that is unsubstituted or substituted with one or more R²⁰ (e.g., C₁₋₆ alkyl that is unsubstituted or substituted with —OH or —CN). In some embodiments, each R^{e} and R^{f} is independently selected from hydrogen and R¹¹, wherein each R¹¹ is independently selected from —OR¹², halogen, a 5-6 membered heteroaryl, and C₁₋₆alkyl that is unsubstituted or substituted with one or more R²⁰. In some embodiments, each R^{e} and R^{f} is hydrogen. It will be appreciated that when R^{e} or R^{f} is ═O, the other R^{e} or R^{f} on the same carbon atom is absent, such that the carbon has proper valency.

In some embodiments, for a compound according to Formula IIIE,

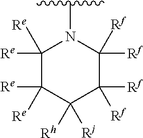

is a structure selected from:

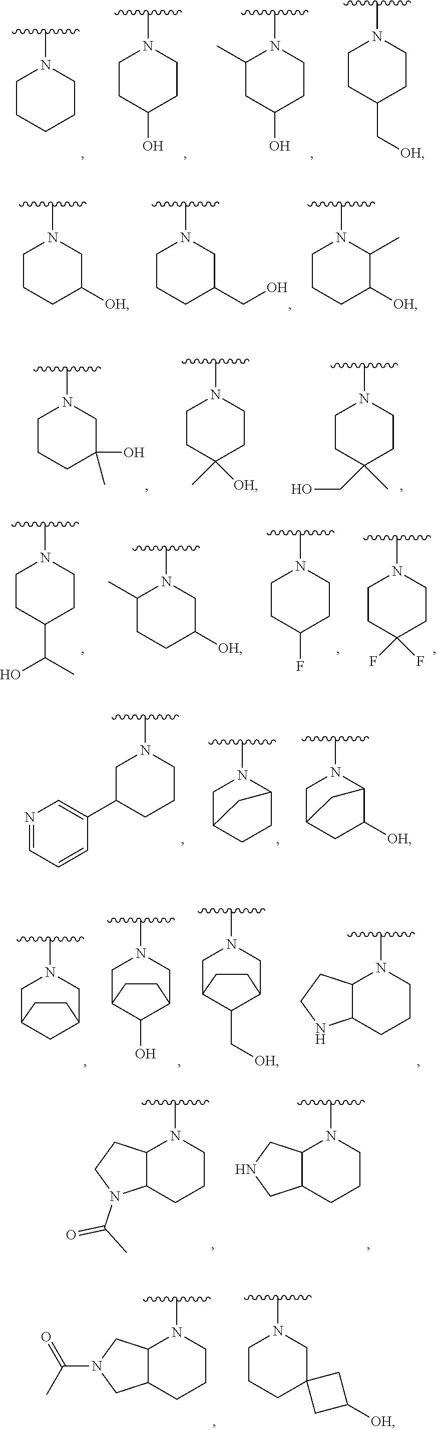

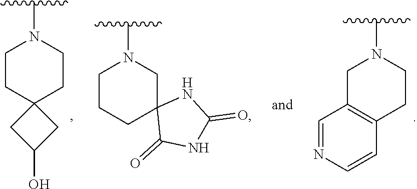

In some embodiments, the compound is a compound according to Formula IIIF, IIIG, or IIIH:

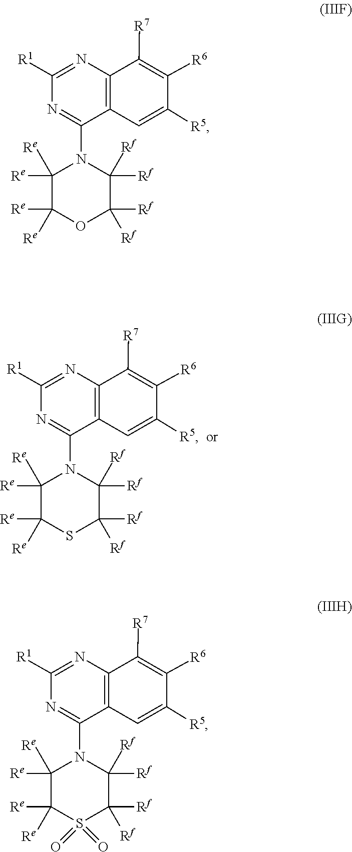

or a salt (e.g., pharmaceutically acceptable salt), ester, tautomer, prodrug, zwitterionic form, or stereoisomer(s) thereof, wherein:
R¹ is selected from —OR⁸ and

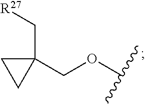

R⁵ is selected from halogen and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety ofany alkylheterocycle is selected from C₁₋₆alkyl;
each R¹¹ is independently selected from deuterium, —OR¹¹, ═O, ═N(R¹⁴), —C(O)(C₁₋₆alkylene)CN, —C(O)(C₁₋₆alkylene)OR¹⁴, —C(O)(C₁₋₆alkyl), —C(O)N(R¹²)₂, —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R¹³;
each R¹² is independently selected from C₁₋₆ alkyl, C₂₋₆ alkenyl, and H, wherein any C₁₋₆alkyl or C₂₋₆alkenyl is unsubstituted or substituted with one or more R¹¹;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹¹;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²⁰;
each R²⁸ is independently selected from C₁₋₆alkyl and halogen;
R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹¹; and
each R^{e} and R^{f} is independently selected from R¹¹ and hydrogen, wherein:
   (i) an R^{e} and an R^{f} can optionally join together to form a 4-6 membered ring;
   (ii) a first R^{f} and a second R^{f} connected to adjacent atoms can optionally join together to form a 3-5 membered ring;
   (iii) a first R^{e} and a second R^{e} connected to adjacent atoms can optionally join together to form a 3-5 membered ring; or
   (iv) a first R^{f} and a second R^{f} connected to the same atom can optionally join together to form a 3-5 membered ring,
   wherein any ring formed by one or more R^{e} and/or one or more R^{f} is unsubstituted or substituted with one or more R¹¹.

In some embodiments, the compound is a compound according to Formula IIIF, or a salt (e.g., pharmaceutically acceptable salt), ester, tautomer, prodrug, zwitterionic form, or stereoisomer(s) thereof. In some embodiments, the compound is a compound according to Formula IIIG, or a salt (e.g., pharmaceutically acceptable salt), ester, tautomer, prodrug, zwitterionic form, or stereoisomer(s) thereof. In some embodiments, the compound is a compound according to Formula IIIH, or a salt (e.g., pharmaceutically acceptable salt), ester, tautomer, prodrug, zwitterionic form, or stereoisomer(s) thereof. In some embodiments, the present disclosure provides a compound of Formula IIIF or a salt (e.g., a pharmaceutically acceptable salt) thereof. In some embodiments, the present disclosure provides a compound of Formula IIIG or a salt (e.g., a pharmaceutically acceptable salt) thereof. In some embodiments, the present disclosure provides a compound of Formula IIIH or a salt (e.g., a pharmaceutically acceptable salt) thereof.

In some embodiments, for a compound according to Formula IIIF, IIIG, or IIIH, each R^{e} and R^{f} is independently selected from R¹¹ and hydrogen. In some embodiments, each R¹¹ is independently selected from deuterium, —OR¹⁴, ═O, ═N(R¹²), —C(O)(C₁₋₆alkylene)CN, —C(O)(C₁₋₆alkylene)OR¹⁴, —C(O)(C₁₋₆alkyl), —C(O)N(R⁴h, —S(O)₂(C₁₋₆alkyl), —N(R¹²)C(O)(C₁₋₆-alkyl), —N(R¹⁴)C(O)(C₁₋₆alkylene)OR¹⁴, halogen, and C₁₋₆ alkyl that is unsubstituted or substituted with one or more R²⁰. In some embodiments, at least one of R^{e} and R^{f} is —OR¹¹ or C₁₋₆alkyl that is unsubstituted or substituted with one or more R¹⁰(e.g., C₁₋₆ alkyl that is unsubstituted or substituted with —OH or —CN). In some embodiments, each R^{e} is hydrogen. In some embodiments, each R^{f} is hydrogen. In some embodiments, each R^{e} and R^{f} is hydrogen. It will be appreciated that when R^{e} or R^{f} is ═O, the other R^{e} or R^{f} on the same carbon atom is absent, such that the carbon has proper valency.

In some embodiments, for a compound according to Formula IIIF, IIIG, or IIIH,

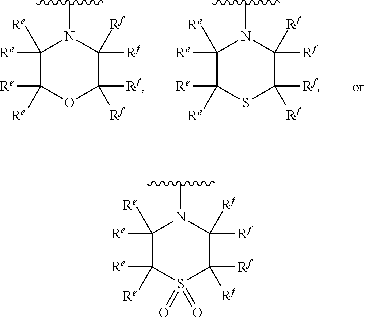

is a structure selected from:

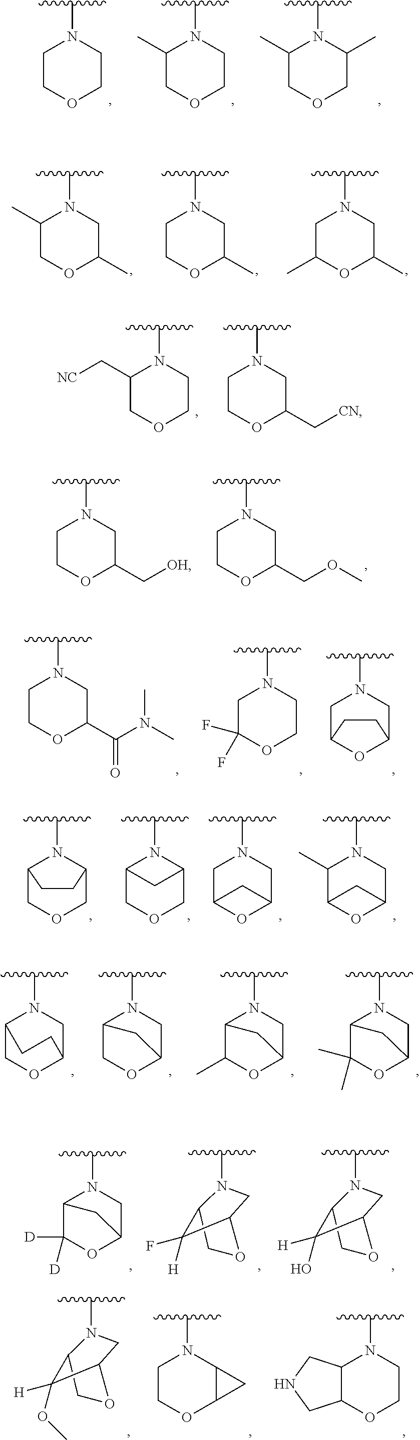

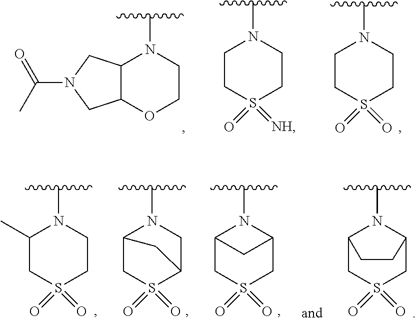

In some embodiments, the compound is a compound according to Formula IIU:

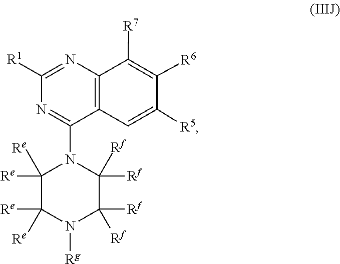

or a salt (e.g., pharmaceutically acceptable salt), ester, tautomer, prodrug, zwitterionic form, or stereoisomer(s) thereof, wherein:
R¹ is selected from —OR⁸ and

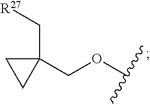

R⁵ is selected from halogen and C₁₋₆alkyl, wherein any C₁₋₆alkyl is unsubstituted or substituted with one or more R¹¹;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety ofany alkylheterocycle is selected from C₁₋₆alkyl;
each R¹¹ is independently selected from deuterium, —OR¹⁴, —O, —N(R¹⁴), —C(O)(C₁₋₆alkylene)CN, —C(O)(C₁₋₆alkylene)OR¹⁴, —C(O)(C₁₋₆alkyl), —C(O)N(R¹²)₂, —C(O)₃₋₆ membered carbocycle or heterocycle), —S(O)₂(C₁₋₆alkyl), —N(R¹²)C(O)(C₁₋₆alkyl), —N(R¹⁴)C(O)(C₁₋₆alkylene)OR¹⁴, halogen, —CN, a 3-6 membered carbocycle or heterocycle, and C₁₋₆alkyl, wherein any C₁₋₆alkyl, carbocycle, or heterocycle is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹²)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹¹;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R¹³;
each R²⁰ is independently selected from C₁₋₆alkyl and halogen;
R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹¹;
each R^{e} and R^{f} is independently selected from R¹¹ and hydrogen, wherein:
   (i) an R^{e} and an R^{f} can optionally join together to form a 4-6 membered ring;
   (ii) a first R^{f} and a second R^{f} connected to adjacent atoms can optionally join together to form a 3-5 membered ring;
   (iii) a first R^{e} and a second R^{e} connected to adjacent atoms can optionally join together to form a 3-5 membered ring; or
   (iv) a first R^{f} and a second R^{f} connected to the same atom can optionally join together to form a 3-5 membered ring,
   wherein any ring formed by one or more R^{e} and/or one or more R^{f} is unsubstituted or substituted with one or more R¹¹; and
R^{g} is R¹¹, or a R^{g} and a R^{e} or a R^{g} and a R^{f} optionally join together to form a 3-6 membered ring that is unsubstituted or substituted with one or more R¹¹.

In some embodiments, the present disclosure provides a compound of Formula IIIJ or a salt (e.g., a pharmaceutically acceptable salt) thereof.

In some embodiments, for a compound according to Formula IIIJ, each R^{e} and R^{f} is independently selected from R¹¹ and hydrogen. In some embodiments, each R¹¹ is independently selected from deuterium, —OR¹², ═O, ═N(R¹²), —C(O)(C₁₋₆alkylene)CN, —C(O)(C₁₋₆alkylene)OR¹⁴, —C(O)(C₁₋₆alkyl), —C(O)N(R¹²)₂, —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl that is unsubstituted or substituted with one or more R²⁰. In some embodiments, each R¹¹ is independently selected from deuterium, —OR¹², ═O, ═N(R¹⁴), —C(O)C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl that is unsubstituted or substituted with one or more R²⁰. In some embodiments, at least one of R^{e} and R^{f} is —OR¹² or C₁₋₆alkyl that is unsubstituted or substituted with one or more R²⁰ (e.g., C₁₋₆alkyl that is unsubstituted or substituted with —OH or —CN). In some embodiments, each R^{e} is hydrogen. In some embodiments, each R^{f} is hydrogen. In some embodiments, each R^{e} and R^{f} is hydrogen. It will be appreciated that when R or R^{f} is ═O, the other R^{e} or R^{f} on the same carbon atom is absent, such that the carbon has proper valency. In some embodiments, an R^{e} and an R^{f} join together to form a 4-6 membered ring.

In some embodiments, for a compound according to Formula IIIJ,

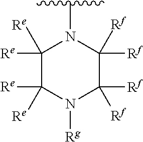

is a structure selected from:

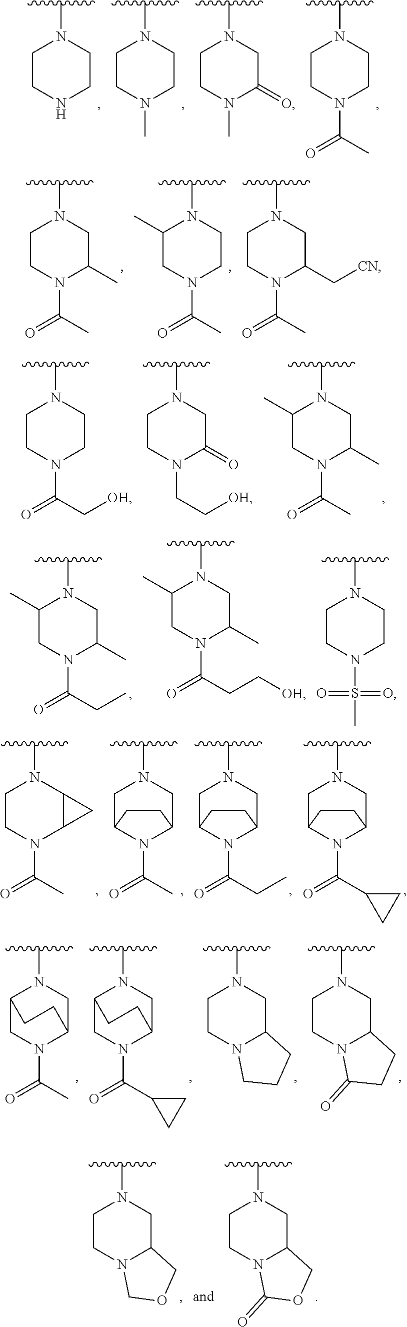

In some embodiments, for a compound according to any one of Formulas IIID, IIIE, IIF, IIIG, IIIH and IIIJ, an R^{e} and an R^{f} join together to form a 4-6 membered ring. In some embodiments, the 4-6 membered ring is a 4-6 membered carbocycle or heterocycle that is unsubstituted. In some embodiments, the 4-6 membered ring is a 4-6 membered carbocycle that is unsubstituted. In some embodiments, the 4-6 membered ring is a 4-6 membered heterocycle that is unsubstituted. In some embodiments, the 4-6 membered ring is a 4-6 membered carbocycle or heterocycle that is substituted with one or more R¹¹. In some embodiments, when an R^{e} and an R^{f} join together to form a 4-6 membered ring, then no additional rings are formed by any combination of R^{e}s and R^{f}s.

In some embodiments, for a compound according to any one of Formulas IIID, IIIE, IIIF, IIIG, IIIH, and IIIU, a first R^{f} and a second R^{f} connected to adjacent atoms join together to form a 3-5 membered ring. In some embodiments, the 3-5 membered ring is a 3-5 membered carbocycle or heterocycle that is unsubstituted. In some embodiments, the 3-5 membered ring is a 3-5 membered carbocycle that is unsubstituted. In some embodiments, the 3-5 membered ring is a 3-5 membered heterocycle that is unsubstituted. In some embodiments, the 3-5 membered ring is a 3-5 membered carbocycle or heterocycle that is substituted with one or more R¹¹. In some embodiments, when a first R^{f} and a second R^{f} connected to adjacent atoms join together to form a 3-5 membered ring, then no additional rings are formed by any combination of R^{e}s and R^{f}s.

In some embodiments, for a compound according to any one of Formulas IIID, IIIE, IIIF, IIIG, IIIH, and IIIJ, a first R^{e} and a second R^{e} connected to adjacent atoms join together to form a 3-5 membered ring. In some embodiments, the 3-5 membered ring is a 3-5 membered carbocycle or heterocycle that is unsubstituted. In some embodiments, the 3-5 membered ring is a 3-5 membered carbocycle that is unsubstituted. In some embodiments, the 3-5 membered ring is a 3-5 membered heterocycle that is unsubstituted. In some embodiments, the 3-5 membered ring is a 3-5 membered carbocycle or heterocycle that is substituted with one or more R¹¹. In some embodiments, when a first R^{e} and a second R^{e} connected to adjacent atoms join together to form a 3-5 membered ring, then no additional rings are formed by any combination of R^{e}s and R^{f}s.

In some embodiments, for a compound according to any one of Formulas IIID, IIIE, IIIF, IIIG, IIIH, and IIIJ, a first R^{f} and a second R^{f} connected to the same atom join together to form a 3-5 membered ring. In some embodiments, the 3-5 membered ring is a 3-5 membered carbocycle or heterocycle that is unsubstituted. In some embodiments, the 3-5 membered ring is a 3-5 membered carbocycle that is unsubstituted. In some embodiments, the 3-5 membered ring is a 3-5 membered heterocycle that is unsubstituted. In some embodiments, the 3-5 membered ring is a 3-5 membered carbocycle or heterocycle that is substituted with one or more R¹¹. In some embodiments, when a first R^{f} and a second R^{f} connected to the same atom join together to form a 3-5 membered ring, then no additional rings are formed by any combination of R^{e}s and R^{f}s.

In some embodiments, for a compound according to Formula IIIJ. R¹ is selected from —OR¹², ═O, —C(O)(C₁₋₆alkylene)CN, —C(O)(C₁₋₆alkylene)OR¹⁴, —C(O)(C₁₋₆alkyl), —C(O)N(R¹²)₂, —S(O)₂(C₁₋₆alkyl), —N(R¹²)C(O)(C₁₋₆alkyl), —N(R¹²)C(O)C₁₋₆alkylene)OR¹⁴, halogen, and C₁₋₆alkyl that is unsubstituted or substituted with one or more R²⁰. In some embodiments, R⁵ is selected from —C(O)(C₁₋₆alkylene)CN, —C(O(C₁₋₆alkylene)OR¹¹, —C(O(C₁₋₆alkyl), —C(O)N(R¹²)₂, —S(O)₂(C₁₋₆alkyl), and C₁₋₆alkyl that is unsubstituted or substituted with one or more R²⁰. In some embodiments, R^{g} is selected from —C(O(C₁₋₆ alkylene)CN, —C(O)(C₁₋₆alkylene)OR¹⁴, —C(O)(C₁₋₆alkyl), —S(O)₂(C₁₋₆alkyl), and C₁₋₆alkyl that is unsubstituted or substituted with one or more R²⁰. In some embodiments, a R^{g} and a R^{e} or a R^{g} and a R^{f} optionally join together to form a 3-6 membered ring that is unsubstituted or substituted with one or more R¹¹.

In some embodiments, the compound is a compound according to Formula IIIK, IIIL, IIIM, IIIN, IIIP, IIIQ, or IIIR

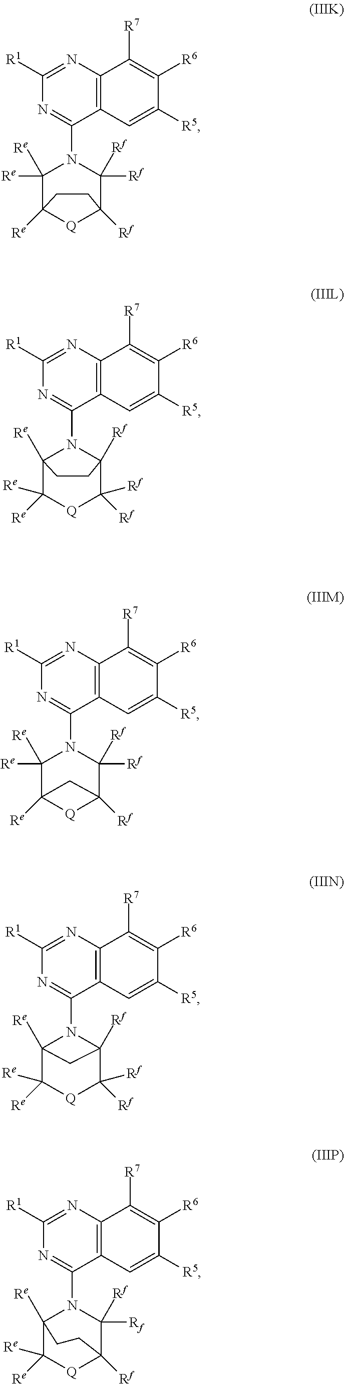

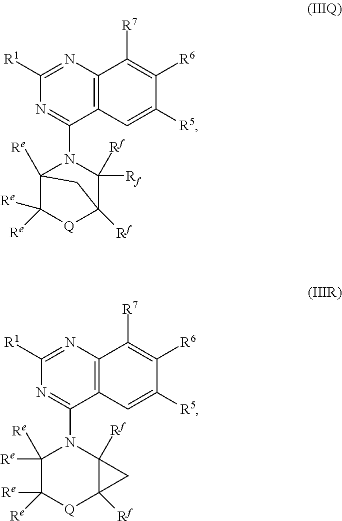

or a salt (e.g., pharmaceutically acceptable salt), ester, tautomer, prodrug, zwitterionic form, or stereoisomer(s) thereof, wherein:
R¹ is selected from —OR⁸ and

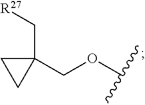

R⁵ is selected from halogen and C₁₋₆alkyl, wherein any C₁₋₆alkyl is unsubstituted or substituted with one or more R¹¹;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹¹ is independently selected from deuterium, —OR¹², ═O, ═N(R¹²), —C(O)(C₁₋₆alkylene)CN, —C(O)(C₁₋₆alkylene)OR¹⁴, —C(O)(C₁₋₆alkyl), —C(O)N(R¹⁴)₂, —C(O)(3-6 membered carbocycle or heterocycle), —S(O)₂(C₁₋₄ alkyl), —N(R¹⁴)C(O)(C₁₋₆alkyl), —N(R¹²)C(O)(C₁₋₆alkylene)OR¹⁴, halogen, —CN, a 3-6 membered carbocycle or heterocycle, and C₁₋₆alkyl, wherein any C₁₋₆alkyl, carbocycle, or heterocycle is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹¹;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen;
R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹¹;
Q is selected from CR^{h}R^{j}, NR^{g}, O, S, and SO₂:
each R^{e} and R^{f} is independently selected from R¹¹ and hydrogen;
R^{g}, when present, is R¹¹, and
R^{h} and R^{j}, when present, are each independently selected from R¹¹ and hydrogen.

In some embodiments, the compound is a compound according to Formula IIIK, or a salt (e.g., pharmaceutically acceptable salt), ester, tautomer, prodrug, zwitterionic form, or stereoisomer(s) thereof. In some embodiments, the compound is a compound according to Formula IIIL, or a salt (e.g., pharmaceutically acceptable salt), ester, tautomer, prodrug, zwitterionic form, or stereoisomer(s) thereof. In some embodiments, the compound is a compound according to Formula IIIM, or a salt (e.g., pharmaceutically acceptable salt), ester, tautomer, prodrug, zwitterionic form, or stereoisomer(s) thereof. In some embodiments, the compound is a compound according to Formula IIIN, or a salt (e.g., pharmaceutically acceptable salt), ester, tautomer, prodrug, zwitterionic form, or stereoisomer(s) thereof. In some embodiments, the compound is a compound according to Formula IIIP, or a salt (e.g., pharmaceutically acceptable salt), ester, tautomer, prodrug, zwitterionic form, or stereoisomer(s) thereof. In some embodiments, the compound is a compound according to Formula IIIQ, or a salt (e.g., pharmaceutically acceptable salt), ester, tautomer, prodrug, zwitterionic form, or stereoisomer(s) thereof. In some embodiments, the compound is a compound according to Formula IIIR, or a salt (e.g., pharmaceutically acceptable salt), ester, tautomer, prodrug, zwitterionic form, or stereoisomer(s) thereof. In some embodiments, the present disclosure provides a compound of Formula IIIK or a salt (e.g., a pharmaceutically acceptable salt) thereof. In some embodiments, the present disclosure provides a compound of Formula IIIL or a salt (e.g., a pharmaceutically acceptable salt) thereof. In some embodiments, the present disclosure provides a compound of Formula IIIM or a salt (e.g., a pharmaceutically acceptable salt) thereof. In some embodiments, the present disclosure provides a compound of Formula IN or a salt (e.g., a pharmaceutically acceptable salt) thereof. In some embodiments, the present disclosure provides a compound of Formula IIIP or a salt (e.g., a pharmaceutically acceptable salt) thereof. In some embodiments, the present disclosure provides a compound of Formula IIIQ or a salt (e.g., a pharmaceutically acceptable salt) thereof. In some embodiments, the present disclosure provides a compound of Formula IIIR or a salt (e.g., a pharmaceutically acceptable salt) thereof.

In some embodiments, for a compound according to any one of Formulas IIIK, IIIL, IIIM, IIIN, IIIP, IIIQ, and IIIR, Q is selected from CR^{h}R^{j}, NR^{g}, and O. In some embodiments, Q is CR^{h}Rⁱ. In some embodiments, Q is CR^{h}R^{j} and R^{h} and Rⁱ are each hydrogen. In some embodiments, Q is CR^{h}Rⁱ, R^{h} is hydrogen, and R^{j} is R¹¹. In some embodiments, Q is CR^{h}R^{j} and R^{h} and R^{j} are each R¹¹. In some embodiments, Q is CR^{h}R^{j}, R^{h} is hydrogen, and R^{j} is selected from —OR¹², ═O, ═N(R¹⁴), —C(O)(C₁₋₆alkylene)CN, —C(O)(C₁₋₆alkylene)OR¹⁴, —C(O)(C₁₋₆alkyl), —C(O)N(R¹⁴)₂, —S(O)₂(C₁₋₆alkyl), —N(R¹²)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R^{a}. In some embodiments, Q is NR^{g}. In some embodiments, Q is NR^{g} and R^{g} is selected from —C(O)(C₁₋₆alkylene)CN, —C(O)(C₁₋₆alkylene)OR¹⁴, —C(O)(C₁₋₆alkyl), —C(O)N(R¹⁴)₂, and C₁₋₆alkyl, wherein any C₁₋₆alkyl is unsubstituted or substituted with one or more R²⁰. In some embodiments, Q is NR^{g} and R^{g} is selected from —C(O)(C₁₋₆alkylene)CN, —C(O)C₁₋₆alkylene)OR¹⁴, —C(O)(C₁₋₆alkyl), and C₁₋₆alkyl that is unsubstituted or substituted with one or more R¹¹. In some embodiments, Q is O. In some embodiments, Q is S or SO₂. In some embodiments, Q is S. In some embodiments, Q is SO₂.

In some embodiments for a compound according to any one of Formulas IIIK, IIIL, IIIM, IIIN, IIIP, IIIQ, and IIIR, each R^{e} and R^{f} is independently selected from R¹¹ and hydrogen, and each R¹¹ is independently selected from deuterium, —OR¹², ═O, ═N(R¹⁴), —C(O)(C₁₋₆alkylene)CN, —C(O)(C₁₋₆ alkylene)OR¹⁴, —C(O)(C₁₋₆alkyl), —C(O)N(R¹²)₂, —S(O)₂(C₁₋₆-alkyl), —N(R¹²)C(O)(C₁₋₆alkyl), —N(R¹²)C(O)(C₁₋₆alkylene)OR¹⁴, halogen, and C₁₋₆alkyl that is unsubstituted or substituted with one or more R²⁰. In some embodiments, each R^{e} and R^{f} is independently selected from R¹¹ and hydrogen, and each R¹¹ is independently selected from —OR¹², —C(O)(C₁₋₆alkylene)CN, —C(O)(C₁₋₆alkylene)OR¹⁴, —C(O)(C₁₋₆alkyl), —C(O)N(R¹⁴)₂, halogen, and C₁₋₆alkyl that is unsubstituted or substituted with one or more R²⁰. In some embodiments, each R^{e} and R^{f} is independently selected from R¹¹ and hydrogen, and each R¹¹ is independently selected from —OR¹², —C(O)(C₁₋₆alkylene)CN, —C(O)C₁₋₆alkylene)OR¹⁴, —C(O)(C₁₋₆alkyl), halogen, and C₁₋₆alkyl that is unsubstituted or substituted with one or more R¹¹. In some embodiments, at least one of R^{e} and R^{f} is —OR¹² or C₁₋₆alkyl that is unsubstituted or substituted with one or more R²⁰ (e.g., C₁₋₆ alkyl that is unsubstituted or substituted with —OH or —CN). In some embodiments, each R^{e} and R^{f} is hydrogen. In some embodiments, each Reis hydrogen. In some embodiments, each R^{f} is hydrogen. It will be appreciated that when R^{e} or R^{f} is —O, the other R^{e} or R^{f} on the same carbon atom is absent, such that the carbon has proper valency.

In some embodiments for a compound according to any one of Formulas IIIK, IIIL, IIIM, IIIN, IIIP, IIIQ, and IIIR, R^{h} and R^{j}, when present, are each independently selected from R¹¹ and hydrogen, and each R¹¹ is independently selected from deuterium, —OR¹², ═O, ═N(R¹⁴), —C(O)(C₁₋₆alkylene)CN, —C(O)(C₁₋₆ alkylene)OR¹⁴, —C(O)(C₁₋₆alkyl), —C(O)N(R¹⁴)₂, —S(O)₂(C₁₋₆alkyl), —N(R¹²)C(O)(C₁₋₆alkyl), —N(R¹⁴)C(O)(C₁₋₆alkylene)OR¹⁴, halogen, and C₁₋₆alkyl that is unsubstituted or substituted with one or more R²⁰. In some embodiments, R^{h} and R^{j}, when present, are each independently selected from R¹¹ and hydrogen, and each R¹¹ is independently selected from —OR¹², —C(O)(C₄alkylene)CN, —C(O)(C₁₋₆alkylene)OR¹⁴, —C(O)(C₁₋₆akyl), —C(O)N(R¹⁴)₂, halogen, and C₁₋₆alkyl that is unsubstituted or substituted with one or more R²⁰. In some embodiments, at least one of R^{h} and R^{j} is —OR² or C₁₋₆alkyl that is unsubstituted or substituted with one or more R²⁰ (e.g., C₁₋₆ alkyl that is unsubstituted or substituted with —OH or —CN). In some embodiments, R^{h} is hydrogen and R^{j} is R¹¹. In some embodiments, R^{h} and R^{j} are each hydrogen. It will be appreciated that when R^{h} or R^{j} is ═O, the R^{h} or R^{j} on the same carbon atom is absent, such that the carbon has proper valency.

In some embodiments, for a compound according to any one of Formulas IIIK, IIIL, IIIM, IIIN, IIIP, IIIQ, and IIIR, R^{g}, when present, is R¹¹, and R¹¹ is selected from —OR¹², ═O, —C(O)(C₁₋₆alkylene)CN, —C(O)(C₁₋₆alkylene)OR¹⁴, —C(O)(C₁₋₆alkyl), —C(O)N(R¹⁴)₂, —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl that is unsubstituted or substituted with one or more R²⁰. In some embodiments, R^{g}, when present, is selected from —C(O)(C₁₋₆alkylene)CN, —C(O)(C₁₋₆ alkylene)OR¹⁴, —C(O)(C₁₋₆alkyl), —C(O)N(R¹⁴)₂, —S(O)₂(C₁₋₆alkyl), and C₁₋₆alkyl that is unsubstituted or substituted with one or more R²⁰. In some embodiments, R⁹, when present, is selected from —C(O)(C₁₋₆alkylene)CN, —C(O)(C₁₋₆alkylene)OR¹⁴, —C(O)(C₁₋₆alkyl), —S(O)₂(C₁₋₆alkyl), and C₁₋₆alkyl that is unsubstituted or substituted with one or more R².

In some embodiments, for a compound according to any one of Formulas IIIK, IIIL, IIIM, IIIN, IIIP, IIIQ, and IIIR, Q is O, and each R^{e} and R^{f} is hydrogen. In some embodiments, Q is O, and at least one R^{e} or R^{f} is R¹¹. In some embodiments, Q is O, and at least one R^{e} or R^{f} is R¹¹, wherein each R¹¹ is independently selected from deuterium, —C(O)(C₁₋₆alkylene)CN, —C(O)(C₁₋₆alkylene)OR¹⁴, —C(O)(C₁₋₆alkyl), —C(O)N(R¹⁴)₂, and C₁₋₆alkyl, wherein any C₁₋₆alkyl is unsubstituted or substituted with one or more R²⁰. In some embodiments, Q is O, and at least one R^{e} or R^{f} is R¹¹, wherein each R¹¹ is independently selected from C₁₋₆alkyl, wherein any C₁₋₆alkyl is unsubstituted or substituted with one or more R²⁰.

In some embodiments, for a compound according to any one of Formulas IIIK, IIIL, IIIM, IIIN, IIIP, IIIQ, and IIIR, Q is NR^{g}; each R^{e} and R^{f} is hydrogen; and R^{g} is selected from —C(O)(C₁₋₆alkylene)CN, —C(O)(C₁₋₆alkylene)OR¹⁴, —C(O)(C₁₋₆akyl), —C(O)N(R¹²)₂, —S(O)₂(C₁₋₆alkyl), and C₁₋₆alkyl that is unsubstituted or substituted with one or more R²⁰. In some embodiments, Q is NR^{g}; at least one R^{e} or R^{f} is R¹¹; and R^{g} is selected from —C(O)(C₁₋₆alkylene)CN, —C(O)(C₁₋₆alkylene)OR¹⁴, —C(O)(C₁₋₆alkyl), —C(O)N(R¹⁴)₂, —S(O)₂(C₁₋₆alkyl), and C_{1-b}alkyl that is unsubstituted or substituted with one or more R²⁰. In some embodiments, Q is NR^{g}; at least one R^{e} or R^{f} is R¹¹; and R^{g} is selected from —C(O)(C₁₋₆alkylene)CN, —C(O)(C₁₋₆alkylene)OR¹⁴, —C(O)(C₁₋₆alkyl), —S(O)₂(C₁₋₆alkyl), and C₁₋₆-alkyl that is unsubstituted or substituted with one or more R²⁰. In some embodiments, Q is NR^{g}; at least one R^{e} or R^{f} is R¹¹, wherein each R¹¹ is independently selected from deuterium, —C(O)(C₁₋₆akylene)CN, —C(O)(C₁₋₆alkylene)OR¹⁴, —C(OC₁₋₆alkyl), —C(O)N(R¹⁴)₂, and C₁₋₆alkyl, wherein any C₄alkyl is unsubstituted or substituted with one or more R²⁰; and R^{g} is selected from —C(O)(C₁₋₆alkylene)CN, —C(O)(C₁₋₆akylene)OR¹⁴, —C(O)(C₁₋₆alkyl), —C(O)N(R¹⁴)₂, —S(O)₂(C₁₋₆alkyl), and C₁₋₆alkyl that is unsubstituted or substituted with one or more R²⁰. In some embodiments, Q is NR^{g}; at least one R^{e} or R^{f} is R¹¹, wherein each R¹¹ is independently selected from C₁₋₆alkyl, wherein any C₁₋₆alkyl is unsubstituted or substituted with one or more R²⁰; and R^{g} is selected from —C(O)(C₁₋₆alkylene)CN, —C(O)(C₁₋₆alkylene)OR¹⁴, —C(O)(C₁₋₆alkyl), —C(O)N(R¹⁴)₂, —S(O)₂(C₁₋₆alkyl), and C₁₋₆alkyl that is unsubstituted or substituted with one or more R²⁰.

In some embodiments, for a compound according to any one of Formulas IIIK, IIIL, IIIM, IIIN, IIIP, IIIQ, and IIIR, Q is CR^{h}R^{j}; each R^{e} and R^{f} is hydrogen; and R^{h} and R^{j} are each independently selected from R¹¹ and hydrogen. In some embodiments, Q is CR^{h}R^{j}; each R^{e} and R^{f} is hydrogen; and R^{h} and R^{j} are each hydrogen. In some embodiments, Q is CR^{h}R^{j}; at least one R^{e} or R^{f} is R¹¹, wherein each R¹¹ is independently selected from deuterium, —C(O)(C₁₋₆alkylene)CN, —C(O)(C₁₋₆alkylene)OR¹⁴, —C(O)(C₁₋₆alkyl), —C(O)N(R¹⁴)₂, and C₁₋₆alkyl, wherein any C₁₋₆alkyl is unsubstituted or substituted with one or more R²⁰; and R^{h} and R^{j} are each independently selected from R¹¹ and hydrogen. In some embodiments, Q is CR^{h}Rⁱ; at least one R^{e} or R^{f} is R¹¹, wherein each R¹¹ is independently selected from deuterium, —C(O)(C₁₋₆alkylene)CN, —C(O)(C₁₋₆alkylene)OR¹⁴, —C(O)(C₁₋₆alkyl), —C(O)N(R¹⁴)₂, and C₁₋₆alkyl, wherein any C₁₋₆alkyl is unsubstituted or substituted with one or more R²⁰; and R^{h} and R^{j} are each hydrogen. In some embodiments, Q is CR^{h}R^{j}; at least one R^{e} or R^{f} is independently selected from deuterium, —C(O)(C₁₋₆alkylene)CN, —C(O)(C₁₋₆alkylene)OR¹⁴, —C(O)(C₁₋₆alkyl), —C(O)N(R¹⁴)₂, and C₁₋₆alkyl, wherein any C₁₋₆alkyl is unsubstituted or substituted with one or more R²⁰; R^{h} is hydrogen; and R^{j} is R¹¹. In some embodiments, Q is CR^{h}R^{j} at least one R^{e} or R^{f} is independently selected from deuterium, —C(O)(C₁₋₆alkylene)CN, —C(O)(C₁₋₆alkylene)OR¹⁴, —C(O)(C₁₋₆akyl), —C(O)N(R¹⁴)₂, and C₁₋₆alkyl, wherein any C₁₋₆ alkyl is unsubstituted or substituted with one or more R²⁰; R^{h} is hydrogen; and R^{j} is selected from deuterium, —C(O)(C₁₋₆alkylene)CN, —C(O)(C₁₋₆alkylene)OR¹⁴, —C(O)(C₁₋₆alkyl), —C(O)N(R¹⁴)₂, and C₁₋₆ alkyl, wherein any C₁₋₆alkyl is unsubstituted or substituted with one or more R²⁰.

In some embodiments, for a compound according to any one of Formulas IIIK, IIIL, IIIM, IIIN, IIIP, IIIQ, and IIIR, Q is SO₂ and each R^{e} and R^{f} is hydrogen. In some embodiments, Q is SO₂ and at least one R^{e} or R^{f} is R¹¹. In some embodiments, Q is SO₂, and at least one R^{e} or R^{f} is R¹¹, wherein each R¹¹ is independently selected from deuterium, —C(O)(C₁₋₆alkylene)CN, —C(O)(C₁₋₆alkylene)OR¹⁴, —C(O)(C₁₋₆alkyl), —C(O)N(R¹⁴)₂, and C₁₋₆alkyl, wherein any C₁₋₆alkyl is unsubstituted or substituted with one or more R²⁰. In some embodiments, Q is SO₂, and at least one R^{e} or R^{f} is R¹¹, wherein each R¹¹ is independently selected from C₁₋₆alkyl, wherein any C₁₋₆alkyl is unsubstituted or substituted with one or more R²⁰.

In some embodiments, for a compound according to any one of Formulas IIIK, IIIL, IIIM, IIIN, IIIP, IIIQ, and IIIR,

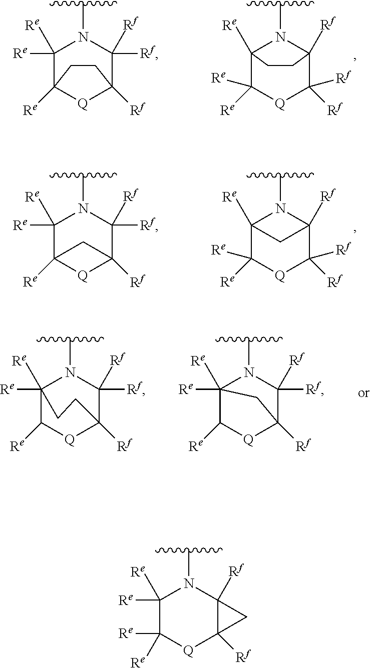

is a structure selected from:

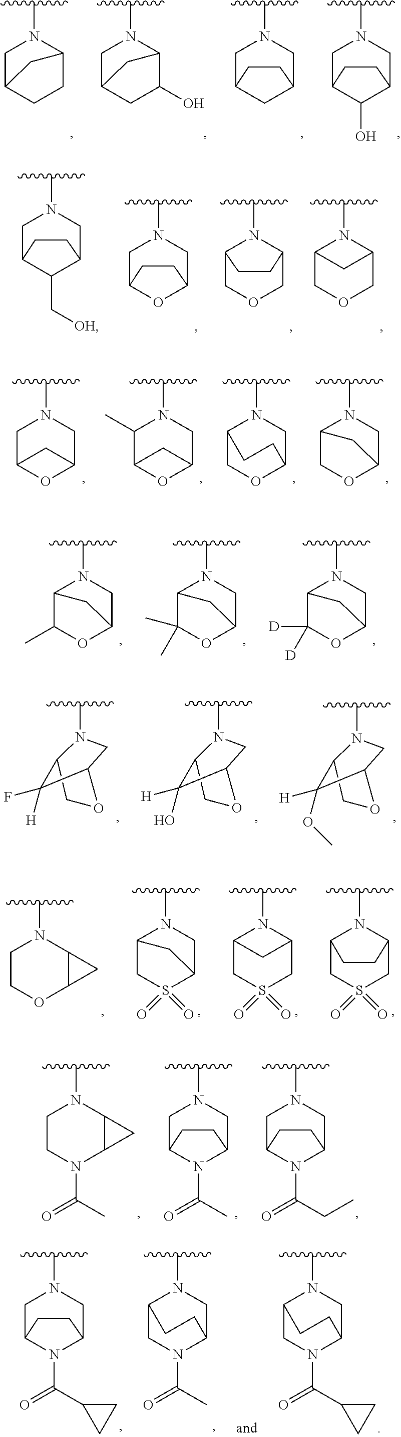

In some embodiments, the compound is a compound according to Formula IIIS:

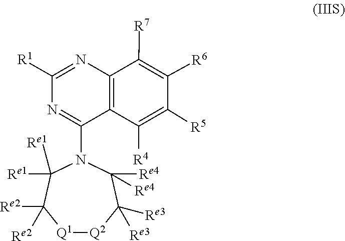

or a salt (e.g., pharmaceutically acceptable salt), ester, tautomer, prodrug, zwitterionic form, or stereoisomer(s) thereof, wherein:
R¹ is selected from —OR⁸ and

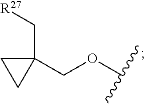

R⁴ is H;
R¹ is selected from halogen and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹² is independently selected from C₁₋₆ alkyl, C₁₋₆ alkenyl, and H, wherein any C₁₋₆alkyl or C₁₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆ alkyl, wherein any C₁₋₆alkyl is unsubstituted or substituted with one or more R¹³;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen;
R^{a} and R^{b} are each independently selected from deuterium, halogen, C₂₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹¹:
Q¹ is selected from NR^{g1}, O, SR^{k}₂, and CRⁱR^{j};
Q² is selected from NR^{g2}, O, SR^{k}₂, and CRⁱR^{j};
R^{g1} and R^{g2}, when present, are independently selected from hydrogen, —C(O)(C₁₋₆alkyl), and C₁₋₆alkyl, wherein any C₁₋₆alkyl is unsubstituted or substituted with one or more R²⁰;
each R^{h} is absent or, when present, is independently selected from ═O, ═N(R¹⁴), and C₁₋₆alkyl, wherein any C₁₋₆alkyl is unsubstituted or substituted with one or more R¹⁰
each Rⁱ and R^{j}, when present, are independently selected from hydrogen, deuterium, —OR¹², ═O, ═N(R¹⁴), —C(O)(C₁₋₆alkylene)CN, —C(O)(C₁₋₆alkylene)OR¹⁴, —C(O)(C₁₋₆alkyl), —C(O)N(R¹⁴)₂, —S(O)h(C₁₋₆alkyl), —N(R¹⁴)C(O)(C₁₋₆ alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰; and
each R^{e1}, R^{e2}, R^{e3}, and R^{e4} are independently selected from hydrogen, deuterium, —OR¹², ═O, ═N(R¹⁴), —C(O)(C₁₋₆alkylene)CN, —C(O)(C₁₋₆alkylene)OR¹⁴, —C(O)(C₁₋₆alkyl), —C(O)N(R¹⁴)₂, —S(O)₂(C₁₋₆ alkyl), —N(R¹⁴)C(O)(C₁₋₆alkyl), —N(R¹⁴)C(O)(C₁₋₆ alkylene)OR¹⁴, halogen, and C₁₋₆ alkyl, wherein any C₁₋₆ alkyl is unsubstituted or substituted with one or more
wherein
   (i) an R^{e2} and an R^{e3} optionally join together to form a 5-6 membered ring; or
   (ii) when Q² is NR^{g2}, R^{g2} and an R^{e3}, together with the atoms to which they are attached, optionally join together to form a 5-membered heterocycle or heteroaryl that is unsubstituted or substituted with one or more R¹¹, wherein each R¹¹ is independently selected from deuterium, —OR¹², ═O, ═N(R¹⁴), —C(O)(C₁₋₆ alkylene)CN, —C(O)(C₁₋₆alkylene)OR¹⁴, —C(O)(C₁₋₆alkyl), —C(O)N(R¹⁴)₂, —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰.

In some embodiments, the present disclosure provides a compound of Formula IIIS or a salt (e.g., a pharmaceutically acceptable salt) thereof.

In some embodiments, for a compound according to Formula ITTS, Q¹ and Q² are each CRⁱR^{j}, wherein each Rⁱ and R^{j} are independently selected from hydrogen, deuterium, —OR¹², —O, —N(R¹⁴), —C(O)(C₁₋₆alkylene)CN, —C(O)(C₁₋₆alkylene)OR¹⁴, —C(O)(C₁₋₆alkyl), —C(O)N(R¹²)₂, —S(O)₂(C₁₋₆alkyl), —N(R¹²)C(O)(C₁₋₆alkyl), —N(R¹⁴)C(O)(C₁₋₆ alkylene)OR¹⁴, halogen, and C₁₋₆ alkyl, wherein any C₁₋₆ alkyl is unsubstituted or substituted with one or more R²⁰. In some embodiments, Q¹ and Q² are each CRⁱR^{j}, and each Rⁱ and R^{j} are independently selected from hydrogen, —C(O)(C₁₋₆alkylene)CN, —C(O)(C₁₋₆ alkylene)OR¹⁴, —C(O)(C₁₋₆alkyl), —C(O)N(R¹⁴)₂, and C₁₋₆alkyl, wherein any C₁₋₆alkyl is unsubstituted or substituted with one or more R²⁰. In some embodiments, Q¹ and Q² are each CRⁱR^{j}, wherein at least one Rⁱ or R^{j} is selected from deuterium, —OR², ═O, ═N(R¹⁴), —C(O)(C₁₋₆alkylene)CN, —C(O)(C₁₋₆alkylene)OR¹⁴, —C(O)(C₁₋₆alkyl), —C(O)N(R¹⁴)₂, —S(O)₂(C₁₋₆alkyl), —N(R¹²)C(O)(C₁₋₆alkyl), —N(R¹²)C(O)(C₁₋₆alkylene)OR¹⁴, halogen, and C₁₋₆alkyl, wherein any C₁₋₆alkyl is unsubstituted or substituted with one or more R¹¹. In some embodiments, Q¹ and Q² are each CRⁱR^{j}, and each Rⁱ and R^{j} are hydrogen. In some embodiments, Q¹ and Q² are each CRⁱR^{j}, each Rⁱ and R^{j} are hydrogen, and at least one R^{e1}, R^{e2}, R^{e3}, or R^{e4} is selected from —OR², ═O, ═N(R¹²), —C(O)(C₁₋₆alkylene)CN, —C(O)(C₁₋₆alkylene)OR¹⁴, —C(O)(C₁₋₆alkyl), —C(O)N(R¹⁴h, —S(O)₂(C₁₋₆alkyl), —N(R¹²)C(O)(C₁₋₆alkyl), —N(R¹²)C(O)(C₁₋₆alkylene)OR¹⁴, halogen, and C₁₋₆alkyl, wherein any C₁₋₆ alkyl is unsubstituted or substituted with one or more R²⁰. In some embodiments, Q¹ and Q² are each CRⁱR^{j}, wherein at least one Rⁱ or R^{j} is selected from deuterium, —OR², ═O, ═N(R¹⁴), —C(O)(C₁₋₆ alkylene)CN, —C(O)(C₁₋₆alkylene)OR¹⁴, —C(O)C₁₋₆alkyl), —C(O)N(R¹⁴)₂, —S(O)₂(C₁₋₆alkyl), —N(R¹⁴)C(O)(C₁₋₆alkyl), —N(R¹⁴)C(O)(C_{1-b}alkylene)OR¹⁴, halogen, and C₁₋₆alkyl, wherein any C₁₋₆alkyl is unsubstituted or substituted with one or more R¹¹; and each R^{c1}, R^{c2}, R^{c3}, and R^{c4} is hydrogen. In some embodiments, Q¹ and Q² are each CRⁱR^{j}, and an Rⁱ and an R^{j} join together to form a 5-6 membered ring.

In some embodiments, for a compound according to Formula IIIS, Q¹ is CRⁱR^{j} and Q² is NR^{g2}. In some embodiments, Q¹ is CRⁱR^{j} and Q² is NR^{g2}, and R^{g2} and an R^{d}, together with the atoms to which they are attached, join together to form a 5-membered heterocycle or heteroaryl that is unsubstituted or substituted with one or more R¹¹. In some embodiments, Q¹ is CRⁱR^{j} and Q² is NR^{g2}, and R^{g2} and an R^{e3}, together with the atoms to which they are attached, join together to form a 5-membered heteroaryl that is unsubstituted or substituted with one or more R¹¹. In some embodiments, Q¹ is CRⁱR^{j} and Q² is NR^{g2}, and R^{g2} and an R^{e3}, together with the atoms to which they are attached, join together to form a 5-membered heteroaryl comprising two nitrogen atoms that is unsubstituted or substituted with one or more R¹¹. In some embodiments, Q¹ is CRⁱR^{j} and Q² is NR^{g2}; R^{g2} and an R^{e3}, together with the atoms to which they are attached, join together to form a 5-membered heterocycle or heteroaryl that is unsubstituted or substituted with one or more R¹¹; and Rⁱ and R^{j} are each hydrogen. In some embodiments, Q¹ is CRⁱR^{j} and Q² is NR^{g2}; R^{g2} and an R^{e3}, together with the atoms to which they are attached, join together to form a 5-membered heterocycle or heteroaryl that is unsubstituted or substituted with one or more R¹¹; and Rⁱ and/or R^{j} is selected from deuterium, —OR², ═O, ═N(R¹⁴), —C(O)(C₁₋₆alkylene)CN, —C(O)(C₁₋₆alkylene)OR¹⁴, —C(O)(C₁₋₆alkyl), —C(O)N(R¹²)₂, —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁-6alkyl is unsubstituted or substituted with one or more R²⁰. In some embodiments, Q¹ is CRⁱR^{j} and Q² is NR^{g2}, and R^{g2} is selected from hydrogen, —C(O)(C₁₋₆alkyl), and C₁₋₆ alkyl, wherein any C₁₋₆alkyl is unsubstituted or substituted with one or more R²⁰. In some embodiments, Q¹ is CRⁱR^{j} and Q² is NR^{g2}; R^{g2} is selected from hydrogen, —C(O)(C₁₋₆alkyl), and C₁₋₆alkyl, wherein any C₁₋₆ alkyl is unsubstituted or substituted with one or more R²⁰; and Rⁱ and R^{j} are each hydrogen. In some embodiments, Q¹ is CRⁱR^{j} and Q² is NR^{g2}; R^{g2} is selected from hydrogen, —C(O)(C₁₋₆alkyl), and C₁₋₆alkyl, wherein any C₁₋₆ alkyl is unsubstituted or substituted with one or more R²⁰; and Rⁱ and/or R^{j} is selected from deuterium, —OR², ═O, ═N(R¹²), —C(O)(C₁₋₆alkylene)CN, —C(O)(C₁₋₆alkylene)OR¹⁴, —C(O)(C₁₋₆alkyl), —C(O)N(R¹²)₂, —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰. In some embodiments, Q¹ is CRⁱR^{j} and Q² is NR^{g2}, and R^{e2} and an R^{e3} join together to form a 5-6 membered ring.

In some embodiments, for a compound according to Formula IIIS, Q¹ is CRⁱR^{j} and Q¹ is NR^{g1}. In some embodiments, Q² is CRⁱR^{j} and Q¹ is NR^{g1}, and R^{g1} is selected from hydrogen, —C(O)(C₁₋₆alkyl), and C₁₋₆alkyl, wherein any C₁₋₆ alkyl is unsubstituted or substituted with one or more R²⁰. In some embodiments, Q² is CRⁱR^{j} and Q¹ is NR^{g1}; R^{g1} is selected from hydrogen, —C(O)(C₁₋₆alkyl), and C₁₋₆alkyl, wherein any C₁₋₆ alkyl is unsubstituted or substituted with one or more R²⁰; and Rⁱ and R^{j} are each hydrogen. In some embodiments, Q² is CRⁱR^{j} and Q¹ is NR^{g1}; R^{g1} is selected from hydrogen, —C(O)(C₁₋₆alkyl), and C₁₋₆alkyl, wherein any C₁₋₆alkyl is unsubstituted or substituted with one or more R¹⁰; and Rⁱ and/or R^{j} is selected from deuterium, —OR¹², ═O, ═N(R¹⁴), —C(O)(C₁₋₆alkylene)CN, —C(O)(C₁₋₆alkylene)OR¹⁴, —C(O)(C₁₋₆ alkyl), —C(O)N(R¹²)₂, —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆ alkyl is unsubstituted or substituted with one or more R²⁰. In some embodiments, Q² is CRⁱR^{j} and Q¹ is NR^{g1}, and an R^{e2} and an R^{e3} join together to form a 5-6 membered ring.

In some embodiments, for a compound according to Formula IIIS, Q¹ is CRⁱR^{j} and Q² is O. In some embodiments, Q¹ is CRⁱR^{j} and Q² is O, wherein Rⁱ and R^{j} are independently selected from hydrogen, deuterium, —OR¹², ═O, ═N(R¹⁴), —C(O)(C₁₋₆alkylene)CN, —C(O)(C₁₋₆alkylene)OR¹⁴, —C(O)(C₁₋₆alkyl), —C(O)N(R¹²)₂, —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆ alkyl, wherein any C₁₋₆ alkyl is unsubstituted or substituted with one or more R²⁰. In some embodiments, Q¹ is CRⁱR^{j} and Q² is O, wherein Rⁱ and R^{j} are independently selected from hydrogen, —C(O)(C₁₋₆ alkylene)CN, —C(O)(C₁₋₆alkylene)OR¹⁴, —C(O)(C₁₋₆alkyl), —C(O)N(R¹⁴)₂, and C₁₋₆alkyl, wherein any C₁₋₄ alkyl is unsubstituted or substituted with one or more R²⁰. In some embodiments, Q¹ is CRⁱR^{j} and Q² is O, and an R^{e2} and an R^{e3} join together to form a 5-6 membered ring.

In some embodiments, for a compound according to Formula IIIS, Q² is CRⁱR^{j} and Q¹ is O. In some embodiments, Q² is CRⁱR^{j} and Q¹ is O, wherein Rⁱ and R^{j} are independently selected from hydrogen, deuterium, —OR¹², —O, —N(R¹⁴), —C(O)(C₁₋₆alkylene)CN, —C(O)(C₁₋₆alkylene)OR¹⁴, —C(O)(C₁₋₆alkyl), —C(O)N(R¹⁴)₂, —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆ alkyl, wherein any C₁₋₆ alkyl is unsubstituted or substituted with one or more R¹¹. In some embodiments, Q² is CRⁱR^{j} and Q¹ is O, wherein Rⁱ and R^{j} are independently selected from hydrogen, —C(O)(C₁₋₆ alkylene)CN, —C(O)(C₁₋₆alkylene)OR¹⁴, —C(O)(C₁₋₆alkyl), —C(O)N(R¹²)₂, and C₁₋₆alkyl, wherein any C₁₋₆ alkyl is unsubstituted or substituted with one or more R²⁰. In some embodiments, Q² is CRⁱR^{j} and Q¹ is O, and an R^{e2} and an R^{e3} join together to form a 5-6 membered ring.

In some embodiments, for a compound according to Formula IIIS, Q¹ is CRⁱR^{j} and Q² is SR^{h}₂. In some embodiments, Q¹ is CRⁱR^{j} and Q² is SR^{h}₂; R¹ and R³ are independently selected from hydrogen, deuterium, —OR¹², ═O, ═N(R¹²), —C(O)(C₁₋₆alkylene)CN, —C(O)(C₄alkylene)OR¹⁴, —C(O)(C₁₋₆ alkyl), —C(O)N(R¹⁴)₂, —S(O)₂(C₁₋₆ alkyl), —N(R¹⁴)C(O)(C₁₋₆alkyl), —N(R¹⁴)C(O)(C₁₋₆-alkylene)OR¹⁴, halogen, and C₁₋₆ alkyl, wherein any C₁₋₆alkyl is unsubstituted or substituted with one or more R¹⁰; and each R^{e} is ═O. In some embodiments, Q¹ is CRⁱR^{j} and Q² is SR^{h}₂; Rⁱ and R^{j} are independently selected from hydrogen, —C(O)(C₁₋₆alkylene)CN, —C(O)(C₁₋₆alkylene)OR¹⁴, —C(O)(C₁₋₆alkyl), —C(O)N(R¹⁴)₂, and C₁₋₆ alkyl, wherein any C₁₋₆alkyl is unsubstituted or substituted with one or more R²⁰; and each R^{h} is ═O. In some embodiments, Q¹ is CRⁱR^{j} and Q² is SR^{h}₂; and an R^{e2} and an R^{e3} join together to form a 5-6 membered ring. In some embodiments, Q¹ is CRⁱR^{j} and Q² is SR^{h}₂; each R^{h} is ═O; and an R^{e2} and an R¹¹ join together to form a 5-6 membered ring.

In some embodiments, for a compound according to Formula IIIS, Q² is CRⁱR^{j} and Q¹ is SR^{h}₂. In some embodiments, Q² is CRⁱR^{j} and Q¹ is SR^{h}₂; Rⁱ and R^{j} are independently selected from hydrogen, deuterium, —OR¹², ═O, ═N(R¹⁴), —C(O)(C₁₋₆alkylene)CN, —C(O)(C₁₋₆alkylene)OR¹⁴, —C(O)C₁₋₆alkyl), —C(O)N(R¹²)₂, —S(O)₂(C₁₋₆ akyl), —N(R¹²)C(O)(C₆alkyl), —N(R¹⁴)C(O)(C₁₋₆alkylene)OR¹⁴, halogen, and C₁₋₆ alkyl, wherein any C₁₋₆alkyl is unsubstituted or substituted with one or more R²⁰; and each R^{h} is ═O. In some embodiments, Q² is CRⁱR^{j} and Q¹ is SR^{h}₂; Rⁱ and R^{j} are independently selected from hydrogen, —C(O)(C₁₋₆alkylene)CN, —C(O)(C₁₋₆ akylene)OR¹⁴, —C(O)(C₁₋₆alkyl), —C(O)N(R¹⁴)₂, and C₁₋₆alkyl, wherein any C₁₋₆alkyl is unsubstituted or substituted with one or more R²⁰; and each R^{h} is ═O. In some embodiments, Q² is CRⁱR^{j} and Q¹ is SR^{h}₂; and an R^{e2} and an R^{e3} join together to form a 5-6 membered ring. In some embodiments, Q² is CRⁱR^{j} and Q¹ is SR^{h}₂; each R^{h} is ═O; and an R^{e2} and an R^{e3} join together to form a 5-6 membered ring.

In some embodiments, for a compound according to Formula IIIS, Q¹ is NR^{g1} and Q² is SR^{h}₂. In some embodiments, Q¹ is NR^{g1} and Q² is SR^{h}₂; each R^{h} is ═O; and R^{g1} is selected from hydrogen, —C(O)(C₁₋₆alkyl), and C₁₋₆alkyl, wherein any C₁₋₆alkyl is unsubstituted or substituted with one or more R¹¹. In some embodiments, Q¹ is NR^{g1} and Q² is SR^{h}₂; each R^{h} is ═O; and R^{g1} is selected from hydrogen, —C(O)(C₁₋₆alkyl), and C₁₋₆ alkyl, wherein any C₁₋₆alkyl is unsubstituted or substituted with one or more R²⁰. In some embodiments, Q¹ is NR^{g1} and Q² is SR^{h}₂; each R^{h} is ═O; R^{g1} is selected from hydrogen, —C(O)(C₁₋₆ alkyl), and C₁₋₆alkyl, wherein any C₁₋₆alkyl is unsubstituted or substituted with one or more R²⁰; and an R^{e2} and an R^{e3} join together to form a 5-6 membered ring.

In some embodiments, for a compound according to Formula IIIS, Q² is NR^{g2} and Q¹ is SR^{h}₂. In some embodiments, Q² is NR^{g2} and Q¹ is SR^{h}₂; each R^{h} is ═O; and R^{g2} is selected from hydrogen, —C(O)(C₁₋₆alkyl), and C₁₋₆alkyl, wherein any C₁₋₆alkyl is unsubstituted or substituted with one or more R²⁰. In some embodiments, Q² is NR^{g2} and Q¹ is SR^{h}₂; each R^{h} is ═O; and R^{g2} is selected from hydrogen, —C(O)(C₁₋₆ alkyl), and C₁₋₆alkyl, wherein any C₁₋₆alkyl is unsubstituted or substituted with one or more R²⁰. In some embodiments, Q² is NR^{g2} and Q¹ is SR^{h}₂; each R^{h} is ═O; R^{g2} is selected from hydrogen, —C(O)(C₁₋₆alkyl), and C₁₋₆alkyl, wherein any C₁₋₆alkyl is unsubstituted or substituted with one or more R²⁰; and an R^{e2} and an R^{e3} join together to form a 5-6 membered ring.

In some embodiments, for a compound according to Formula IIIS,

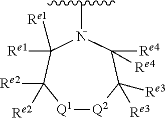

has a structure selected from

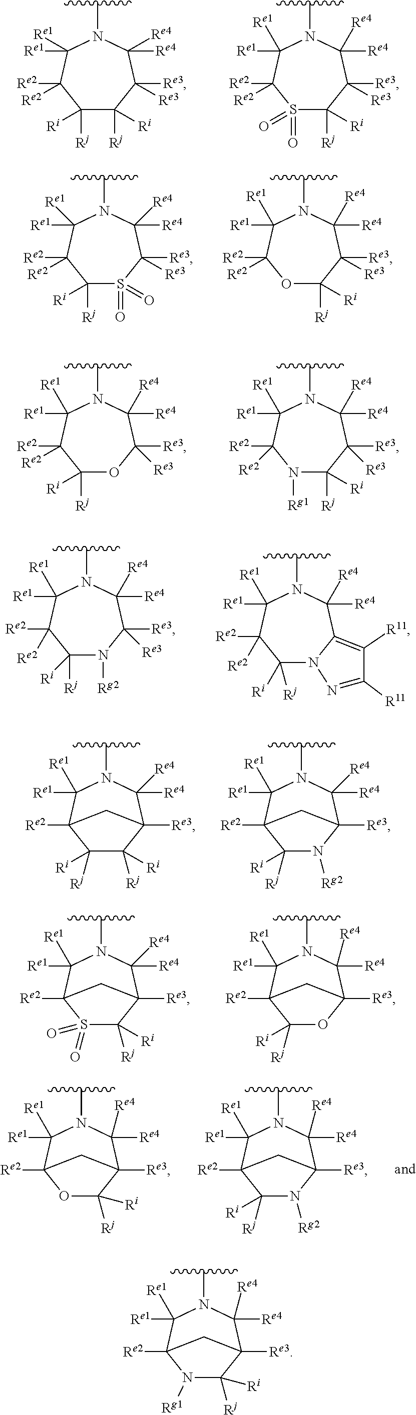

In some embodiments, for a compound according to Formula IIIS,

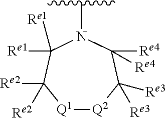

is a structure selected from:

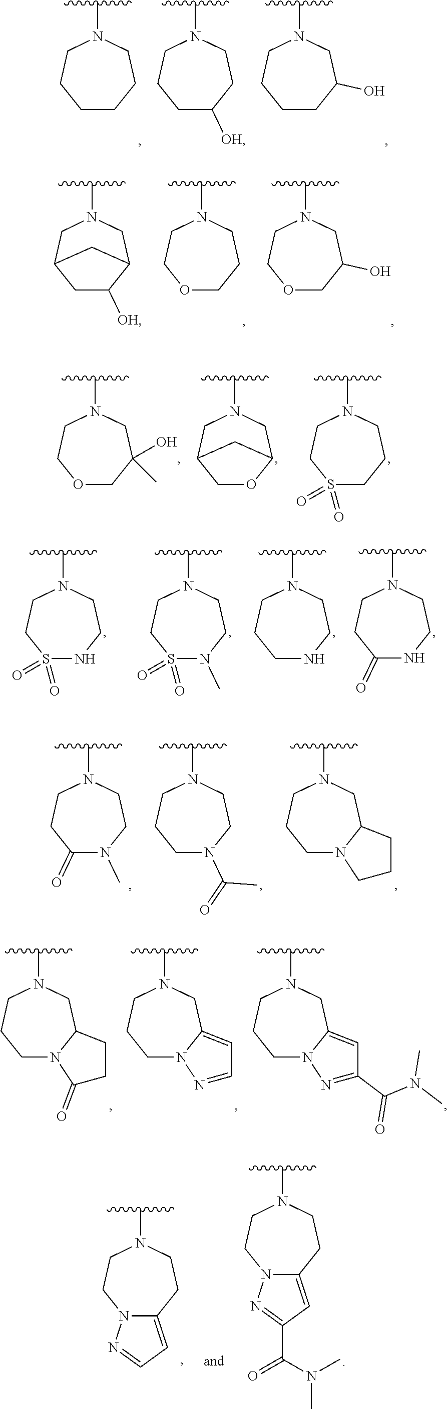

In all of the preceding embodiments for a compound according to Formula IIIS, it will be appreciated that when an R^{e}, R^{g}, Rⁱ, R^{j}, or R¹¹ is ═O, another substitutent that would otherwise be on the same carbon atom is absent, such that the carbon has proper valency.

In some embodiments, the compound is a compound according to Formula IIIT:

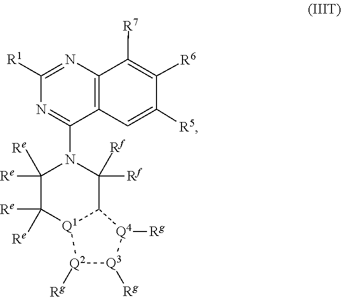

or a salt (e.g., pharmaceutically acceptable salt), ester, tautomer, prodrug, zwitterionic form, or stereoisomer(s) thereof wherein:
R¹ is selected from —OR⁸ and

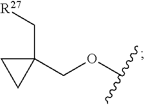

R⁵ is selected from halogen and C₁₋₆alkyl, wherein any C₁₋₆alkyl is unsubstituted or substituted with one or more R¹¹;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹¹ is independently selected from deuterium, —OR¹², ═O, ═N(R¹²), —C(O)(C₁₋₆ alkylene)CN, —C(O)(C₁₋₆ alkylene)OR¹⁴, —C(O)(C₁₋₆ alkyl), —C(O)N(R¹⁴)₂, —C(O)(3-6 membered carbocycle or heterocycle), —S(O)₂(C₁₋₆alkyl), —N(R¹⁴)C(O)(C₁₋₆alkyl), —N(R¹⁴)C(O)(C₁₋₆alkylene)OR¹⁴, halogen, —CN, a 3-6 membered carbocycle or heterocycle, and C₁₋₆alkyl, wherein any C₁₋₆alkyl, carbocycle, or heterocycle is unsubstituted or substituted with one or more R²⁰;
each R¹² is independently selected from C₁₋₆ alkyl, C₂₋₆ alkenyl, and H, wherein any C₁₋₆ alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹³;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen;
R^{a} and R^{b} are each independently selected from deuterium, halogen, C₁₋₆ alkyl, a 3-6 membered carbocycle, —OR¹², and H, wherein an R and R optionally join together to form a 3-6 membered carbocycle or heterocycle, and wherein any C₁₋₆alkyl or 3-6 membered carbocycle or heterocycle is unsubstituted or is substituted with one or more R¹¹, and;
dashed lines represent single or double bonds, such that the ring containing Q¹, Q², Q³, and Q⁴ is aromatic;
Q¹, Q², Q³, and Q¹ are independently selected from C, N, O, and S, wherein at least one of Q¹, Q², Q³, and Q¹ is N;
each R^{e} and R^{f} is independently selected from R¹¹ and hydrogen; and
each R^{g} is absent or is independently selected from R¹¹ and hydrogen.

In some embodiments, the present disclosure provides a compound of Formula IIIT or a salt (e.g., a pharmaceutically acceptable salt) thereof.

In some embodiments, for a compound according to Formula IIIT, Q¹ is C. In some embodiments, Q¹ and Q² are C. In some embodiments, Q¹ and Q² are C, and Q³ and Q¹ are N. In some embodiments, Q¹ is C, Q²-R^{g} is CH, Q³-R^{g} is N, and Q¹-R^{g} is NH. In some embodiments, Q¹ is C, Q²-R^{g} is CH, Q³-R^{g} is N, and Q⁴-R^{g} is N(C₁₋₆alkyl).

In some embodiments, for a compound according to Formula IIIT, Q¹ is N. In some embodiments, Q¹ and Q² are N. In some embodiments, Q¹ and Q² are N, and Q¹ and Q² are C. In some embodiments, Q¹ is N, Q²-R^{g} is N, Q³-R^{g} is CH, and Q⁴-R^{g} is CH. In some embodiments, Q¹ is N, Q²-R^{g} is N, Q³-R^{g} is CC(O)N(R¹⁴)₂, and Q⁴-R^{g} is CH. In some embodiments, Q¹ is N, Q²-R^{g} is N, Q³-R^{e} is CH, and Q⁴-R^{E} is CC(O)N(R¹⁴)₂. In some embodiments, Q¹, Q², and Q³ are N, and Q; is C. In some embodiments, Q¹ is N, Q²-R^{g} and Q³-R^{g} are N, and Q⁴-R¹ is CH. In some embodiments, Q¹, Q³, and Q⁴ are N, and Q² is C. In some embodiments. Q¹ is N, Q³-R^{g} and Q¹-R^{g} are N, and Q²-R^{g} is CH. In some embodiments, Q¹, Q², and Q⁴ are N, and Q³ is C. In some embodiments, Q¹ is N, Q²-R^{g} and Q¹-R^{g} are N, and Q¹-R^{g} is CH. In some embodiments, Q¹ is N, Q²-R^{g} and Q⁴-R^{g} are N, and Q³-R^{g} is CC(O)N(R¹⁴)₂.

In some embodiments, for a compound according to Formula IIT, each R^{g} is hydrogen or is absent. In some embodiments, at least one R^{g} is R¹¹.

In some embodiments, for a compound according to Formula IIIT,

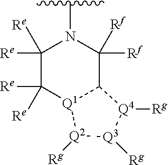

has a structure selected from

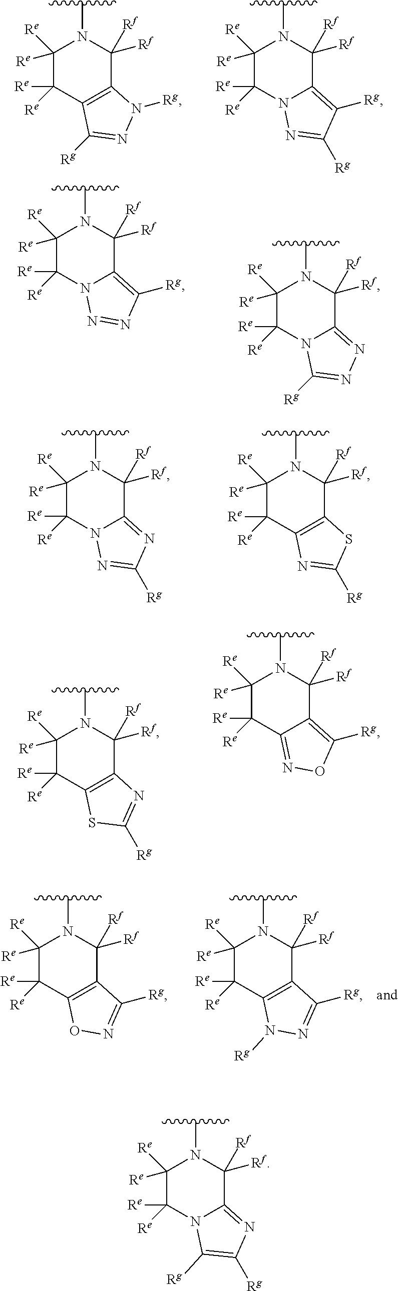

In some embodiments, for a compound according to Formula IIT, at least one R^{g} is R¹¹. In some embodiments, the at least one R^{g} is selected from deuterium, —OR², ═O, ═N(R¹⁴), —C(O)(C₁₋₆alkylene)CN, —C(O)(C₁₋₆ alkylene)OR¹⁴, —C(O)(C₁₋₆ alkyl), —C(O)N(R¹²)₂, —C(O)(3-6 membered carbocycle or heterocycle), —S(O)₂(C₁₋₆ alkyl), —N(R¹²)C(O)(C₁₋₆alkyl), —N(R¹²)C(O)(C₁₋₆ alkylene)OR¹⁴, halogen, —CN, a 3-6 membered carbocycle or heterocycle, and C₁₋₆alkyl, wherein any C₁₋₆ alkyl, carbocycle, or heterocycle is unsubstituted or substituted with one or more R²⁰. In some embodiments, the at least one R^{g} is selected from deuterium, —OR¹², —O, ═N(R¹²), —C(O)(C₁₋₆alkylene)CN, —C(O)(C₁₋₆alkylene)OR¹⁰, —C(O)(C₁₋₆alkyl), —C(O)N(R¹⁴), —S(O)₂(C₁₋₆alkyl), —N(R¹²)C(O)(C₁₋₆alkyl), —N(R¹⁴)C(O)(C₁₋₆alkylene)OR¹⁴, halogen, and C₁₋₆ alkyl, wherein any C₁₋₆alkyl is unsubstituted or substituted with one or more R²⁰. In some embodiments, the at least one R⁶ is selected from deuterium, —OR¹², —C(O)(C₁₋₆alkylene)CN, —C(O)(C₁₋₆alkylene)OR¹⁴, —C(O)(C₁₋₆alkyl), —C(O)N(R¹²)₂, halogen, and C₁₋₆alkyl, wherein any C₁₋₆alkyl is unsubstituted or substituted with one or more R²⁰. In some embodiments, the at least one R¹ is selected from —OR¹², —C(O)(C₁₋₆alkylene)CN, —C(O)(C₁₋₆alkylene)OR¹⁰, —C(O)(C₁₋₆alkyl), —C(O)N(R¹²)₂, and C₁₋₆alkyl, wherein any C₁₋₆ alkyl is unsubstituted or substituted with one or more R²⁰. In some embodiments, the at least one R^{g} is selected from —OR¹², —C(O)(C₁₋₆alkylene)CN, —C(O)(C₁₋₆akylene)OR¹⁴, —C(O)(C₁₋₆alkyl), and C₁₋₆alkyl that is unsubstituted or substituted with one or more R²⁰.

In some embodiments, for a compound according to Formula IIIT,

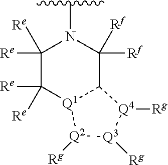

is a structure selected from:

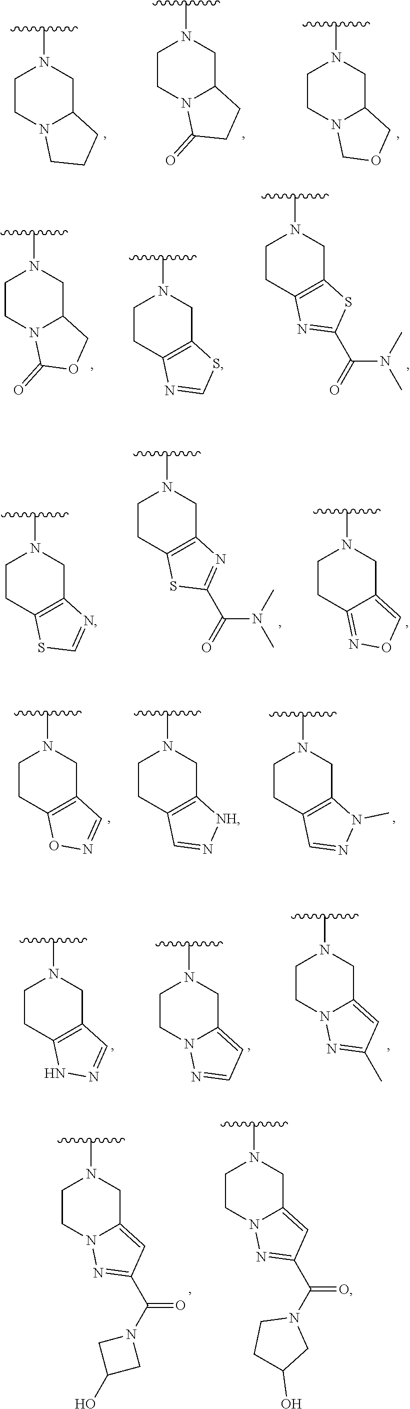

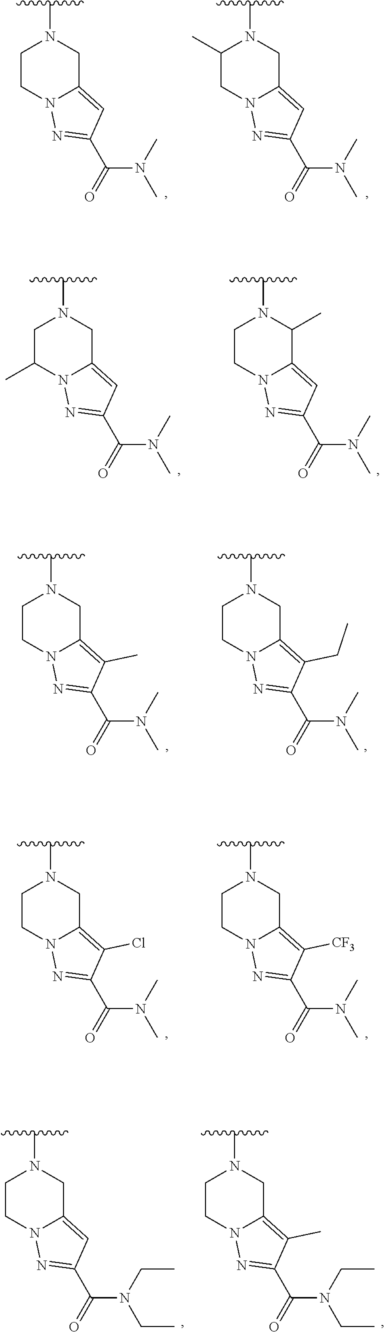

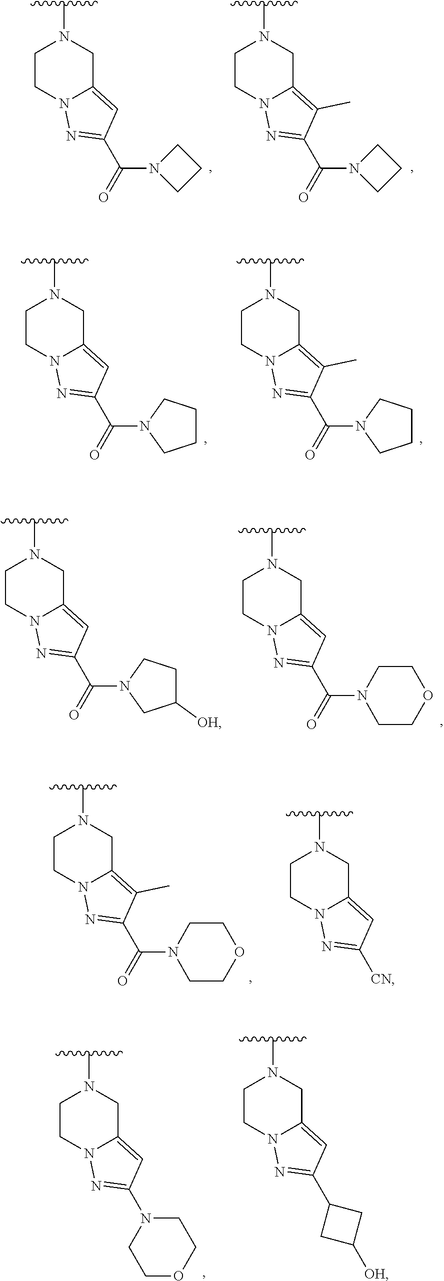

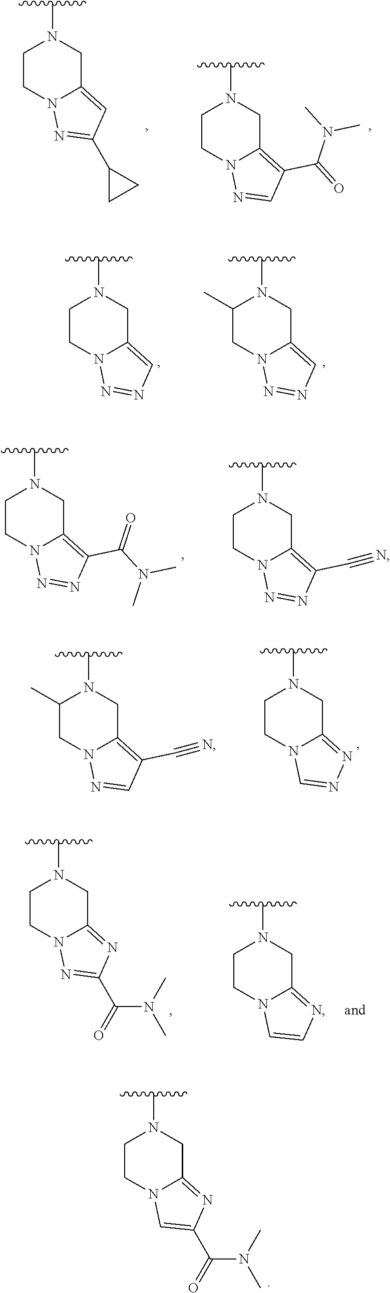

In all of the preceding embodiments for a compound according to Formula IIIT, it will be appreciated that when an R^{e}, R^{f}, or R^{g} is ═O, another substitutent that would otherwise be on the same carbon atom is absent, such that the carbon has proper valency. Similarly, an R¹¹ substituent on Q², Q³, or Q⁴ may be absent to ensure that a corresponding nitrogen atom has proper valency.

In some embodiments, for a compound according to anyone of Formulas III, III′, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, and IIIT, R⁶ is a bicyclic heteroaryl that is substituted with one or more R¹⁵. In some embodiments, R⁶ is selected from:

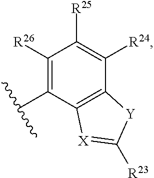

wherein X is selected from N and C—CN; Y is selected from O and S; R²³ is selected from —N(R¹²)₂, C₁₋₆ alkyl, and C₁₋₆alkyl-N(R¹²)₂, wherein any C₁₋₆alkyl is unsubstituted or substituted with one or more R¹¹; and R²⁴, R²⁵, and R²⁶ are independently selected from H, deuterium, halogen, and C₁₋₆alkyl, wherein any C₁₋₆ alkyl is unsubstituted or substituted with one or more R¹¹. In some embodiments, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R¹²)₂. In some embodiments, X is C—CN, Y is S, and R²³ is —NH₂. In some embodiments, R²⁴ is halogen (e.g., fluoro). In some embodiments, R¹¹ is deuterium.

In some embodiments, for a compound according to any one of Formulas III, III′, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, and IIIT, R⁶ is selected from:

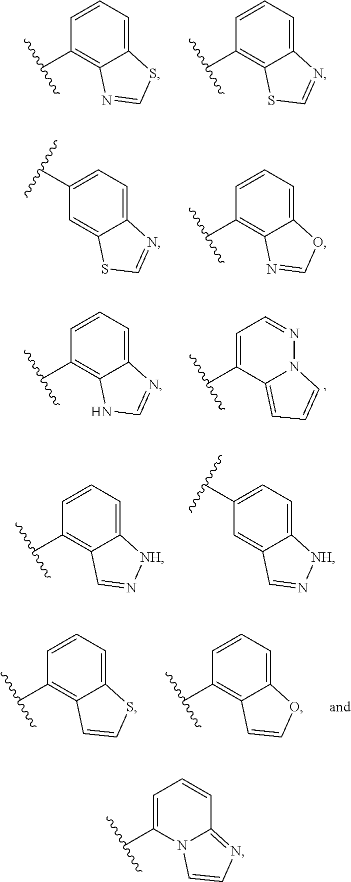

any of which is substituted with one or more R¹¹.

In some embodiments, for a compound according to anyone of Formulas III, III′, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, and IIIT, R⁶ is selected from:

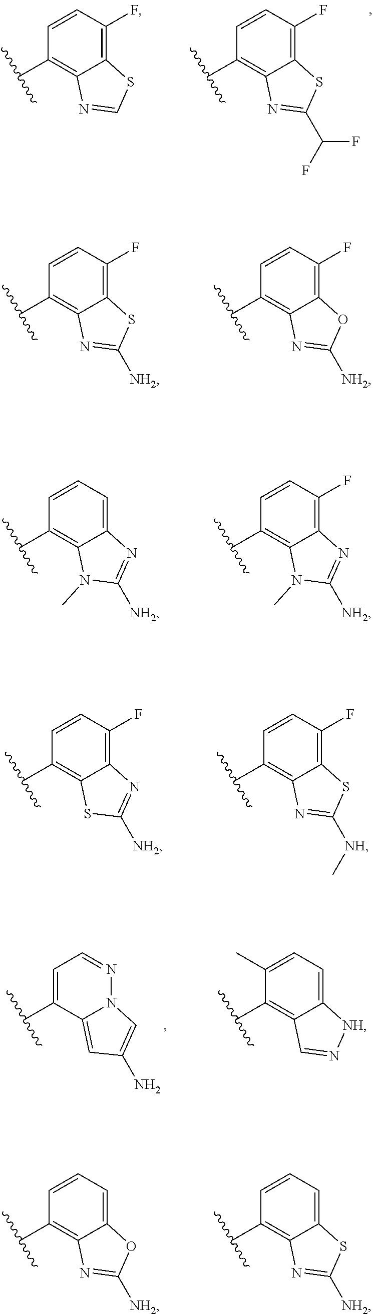

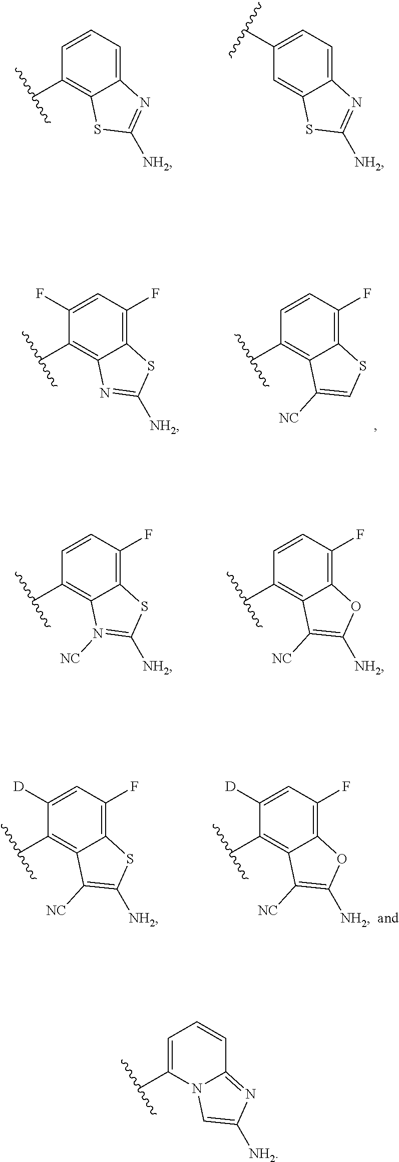

In some embodiments, for a compound according to anyone of Formulas III, III′, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, and IIIT, R⁶ is selected from:

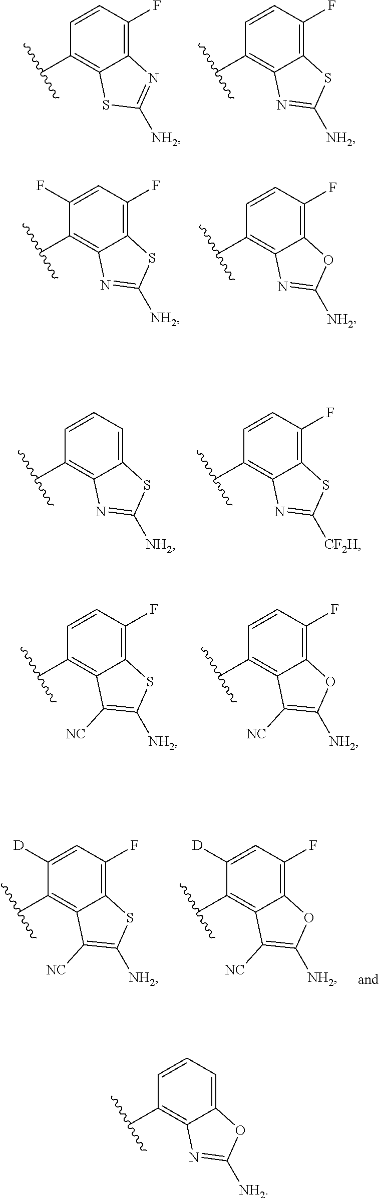

in some embodiments, for a compound according to anyone of Formulas III, III′, IIIA, IIIA1, IIIB, IIIC, IIIC1, IIID, IIIE, IIIF, IIIG, IIIH, IIIJ, IIK, IIIL, IIIM, IIIN, IIIP, IIIQ, IIIR, IIIS, and IIIT, R⁶ is

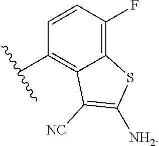 [SMILES: *c1ccc(F)c2sc(N)c(C#N)c12]

In some embodiments, for a compound according to anyone of Formulas III, III′, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, and IIIT, R⁶ is a monocyclic heteroaryl that is substituted with one or more R¹⁵. In some embodiments, R⁶ is a pyridine substituted with one or more R¹⁵. In some embodiments, at least one R¹⁵ is selected from C₁₋₆alkyl, wherein any C₁₋₆alkyl is unsubstituted or substituted with one or more R¹³. In some embodiments, at least one R¹⁵ is —N(R¹²)₂. In some embodiments, R⁶ has a structure selected from

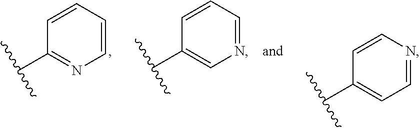 [SMILES: *c1ccccn1 ; *c1cccnc1 ; "and" ; *c1ccncc1]

and any of which is optionally substituted with one or more R¹⁵. In some embodiments, R⁶ has the structure

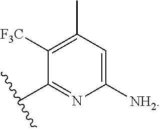 [SMILES: *c1nc(N)cc(C)c1C(F)(F)F]

In some embodiments, for a compound of any one of Formulas III, III′, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, and IIIT, R¹ is selected from —OR⁸, wherein R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl In some embodiments, R⁸ is a heterocycle or an alkylheterocycle, wherein any heterocycle contains 4-8 members and is substituted with one or more R^{a} or R^{b}. In some embodiments, R⁸ is a heterocycle that is unsubstituted or substituted with one or more R^{a} or R^{b}. In some embodiments, R⁸ is an alkylheterocycle that is unsubstituted or substituted with one or more R^{a} or R^{b}. In some embodiments, R⁸ is —CH₂(heterocycle), where the heterocycle is unsubstituted or substituted with one or more R^{a} or R^{b}. In some embodiments, a heterocycle or a heterocycle of an alkylheterocycle is a 4-6 membered monocyclic heterocycle having 1-2 heteroatoms independently selected from N, O, and S. In some embodiments, a heterocycle or a heterocycle of an alkylheterocycle is an 8-membered bicyclic heterocycle having 1-2 heteroatoms independently selected from N, O, and S. In some embodiments, a heterocycle or a heterocycle of an alkylheterocycle is substituted with one or more R^{a} or R^{b}, wherein the one or more R^{a} or R^{b} is a halogen (e.g., F). In some embodiments, a heterocycle or a heterocycle of an alkylheterocycle is substituted with one or more R^{a} or R^{b}, wherein the one or more R^{a} or R^{b} is a C₁₋₆alkyl (e.g., methyl). In some embodiments, a heterocycle or a heterocycle of an alkylheterocycle is substituted with one or more R^{a} or R^{b}, wherein the one or more R^{a} or R^{b} is a —OR¹² (e.g., —OCH₃). In some embodiments, a heterocycle or a heterocycle of an alkylheterocycle is substituted with one or more R^{a} or R^{b}, wherein the one or more R^{a} or R^{b} is a 3-6 membered carbocycle (e.g., a cyclopropane). In some embodiments, a heterocycle or a heterocycle of an alkylheterocycle is substituted with one or more R^{a} or R^{b}, wherein the one or more R^{a} or R^{b} is deuterium.

In some embodiments, for a compound of any one of Formulas III, III, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, and IIIT, R¹ is selected from:

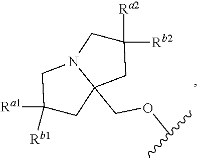

wherein R^{a1}, R^{a2}, R^{b1}, and R^{b2} are each independently selected from deuterium, halogen, C₁₋₆alkyl, —OR¹², and H, wherein R^{a2} and R^{b2} can optionally join together to form a 3-6 membered carbocycle, and wherein any C₁₋₆alkyl or 3-6 membered carbocycle is unsubstituted or is substituted with one or more R¹¹. In some embodiments, R^{a1} and/or R^{a2} is a halogen. In some embodiments, R^{a1} and/or R^{a2} is F. In some embodiments, R^{a1} and/or R^{a2} is C₁₋₆alkyl that is unsubstituted or is substituted with one or more R¹³. In some embodiments, R^{a1} and/or R^{a2} is methyl. In some embodiments, R^{a1} and/or R^{a2} is —OC₁₋₆alkyl. In some embodiments, R^{a1} and/or R^{a2} is H. In some embodiments, R^{b1} and/or R^{b2} is H. In some embodiments, R^{b1} and/or R^{b2} is a halogen. In some embodiments, R^{b1} and/or R^{b2} is F. In some embodiments, R^{b1} and/or R^{b2} is C₁₋₆alkyl that is unsubstituted or is substituted with one or more R¹³. In some embodiments, R^{b1} and/or R^{b2} is methyl. In some embodiments, each of R^{a1} and R^{b1} is F. In some embodiments, R¹ and/or R^{b2} is D. In some embodiments, R^{a2} and R^{b2} join together to form a 3-6 membered carbocycle (e.g., cyclopropane), which carbocycle is optionally substituted with one or more R¹¹. In some embodiments, R^{a1} and R^{b1} join together to form a 3-6 membered carbocycle (e.g., cyclopropane). In some embodiments, R¹ is selected from:

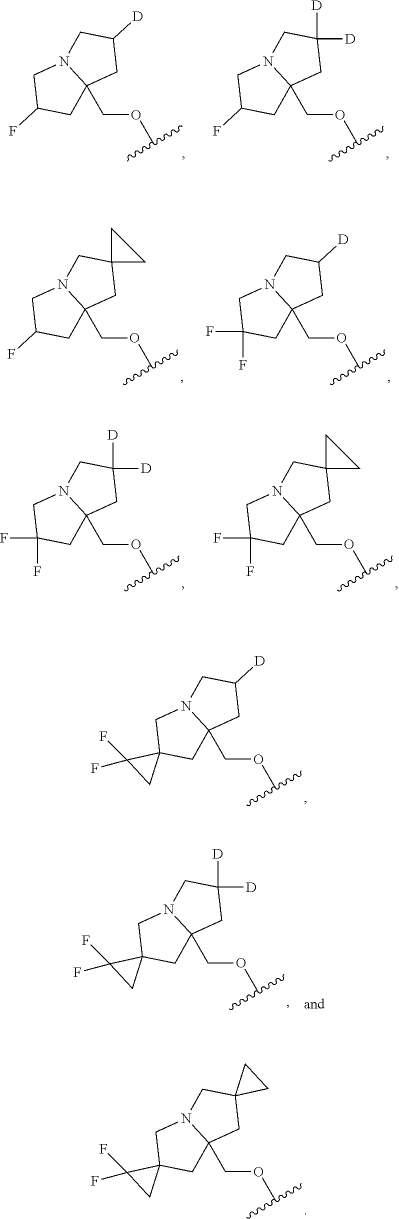

In some embodiments, for a compound of any one of Formulas III, III′, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, and IIIT, R¹ is selected from:

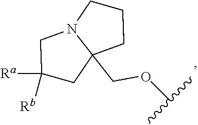

wherein R^{a} and R^{b} are each independently selected from halogen, C₁₋₆alkyl, —OR¹², and H, wherein any C₁₋₆ alkyl is unsubstituted or is substituted with one or more R¹³. In some embodiments, R^{a} is a halogen. In some embodiments, R^{a} is F. In some embodiments, R^{a} is C₁₋₆alkyl that is unsubstituted or is substituted with one or more R¹³. In some embodiments, R^{a} is methyl. In some embodiments, R^{a} is —OC₁₋₆alkyl. In some embodiments, R^{a} is H. In some embodiments, R^{b} is H. In some embodiments, R^{b} is a halogen. In some embodiments, R^{b} is F. In some embodiments, R^{b} is C₁₋₆alkyl that is unsubstituted or is substituted with one or more R¹¹. In some embodiments, R^{b} is methyl. In some embodiments, each of R^{a} and R^{b} is F. In some embodiments, each of R^{a} and R^{b} is methyl. In some embodiments, R¹ is selected from:

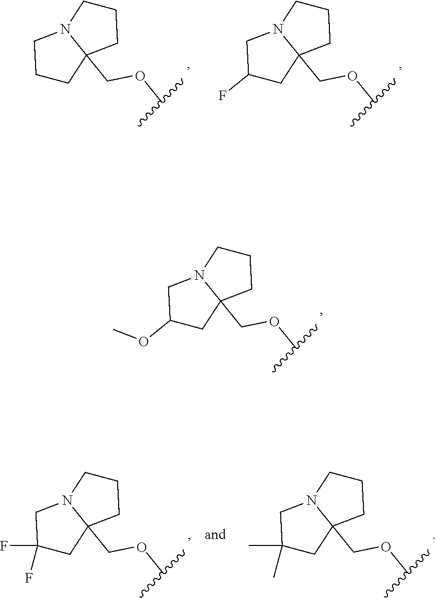 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

In some embodiments, for a compound of any one of Formulas III, III′, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, and IIIT, R¹ is selected from:

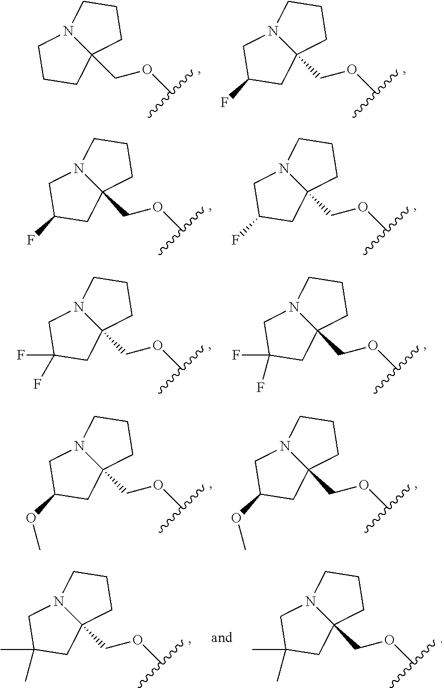

In some embodiments, for a compound of any one of Formulas III, III, IIIA, IIIA1, IIIB, IIIC, IIIC1, IIID, IIIE, IIIF, IIIG, IIIH, IIIU, IIIK, IIIL, IIIM, IIIN, IIIP, IIIQ, IIIR, IIIS, and IIIT, R^{e} is selected from.

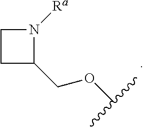

In some embodiments, R^{a} is C₁₋₆alkyl that is unsubstituted or substituted with one or more R¹³. In some embodiments, R^{a} is methyl. In some embodiments, R¹ is selected from:

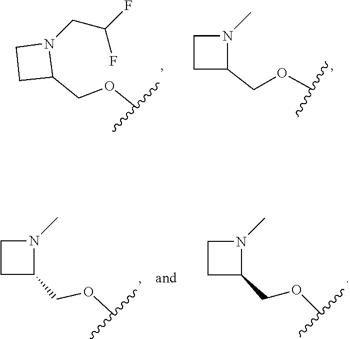

In some embodiments, for a compound of any one of Formulas III, III′, IIIA, IIIA1, IIIB, IIIC, IIIC1, IIID, IIIE, IIIF, IIIG, IIIH, IIIJ, IIIK, IIL, IIIM, IIIN, IIIP, IIIQ, IIIR, IIIS, and IIIT, R¹ is selected from:

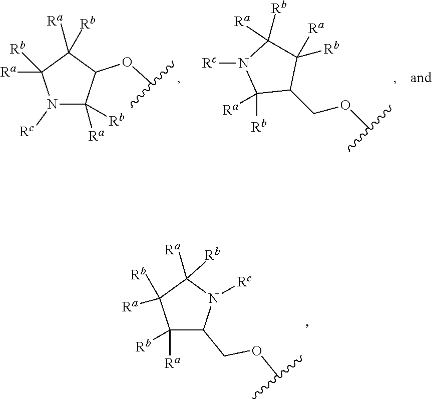

wherein each R^{a} and R^{b} is independently selected from halogen, C₁₋₆ alkyl, —OR¹⁴, and H; and R^{c} is selected from C₁₋₆ alkyl, wherein an R^{a} and R^{b} or R^{c} optionally join together to form a 3-6 membered carbocycle or heterocycle, and wherein any C₁₋₆ alkyl or 3-6 membered carbocycle or heterocycle is unsubstituted or is substituted with one or more R¹³. In some embodiments, each R^{a} and R^{b} is independently selected from halogen, C₁₋₆ alkyl, —OR¹², and H; and R^{c} is selected from C₁₋₆ alkyl, wherein any C₁₋₆ alkyl is unsubstituted or is substituted with one or more R¹³, and wherein an R^{a} and R^{b} attached to the same carbon atom join together to form a 3-6 membered carbocycle. In some embodiments, each R^{a} and R^{h} is independently selected from halogen, C₁₋₆ alkyl, —OR¹², and H; and wherein an R^{a} and R^{c} join together to form a 3-6 membered heterocycle. In some embodiments, each R^{a} and R^{b} is independently selected from halogen, C₁₋₆ alkyl, —OR¹⁴, and H; and R²⁰ is selected from C₁₋₆ alkyl, wherein any C₁₋₆alkyl is unsubstituted or is substituted with one or more R¹². In some embodiments, one R^{a} or R^{b} is selected from halogen, C₁₋₆alkyl, and —OR¹², and the other R^{a} and R^{b} groups are H. In some embodiments, one R^{a} or R^{b} is halogen (e.g., F). In some embodiments, two R^{a} groups, two R^{b} groups, or an R^{a} and an R^{b} are halogen (e.g., F). In some embodiments, one R^{a} or R^{b} is —OR¹² (e.g., —OCH₃ or —OCHF₂). In some embodiments, one R^{a} or R^{b} is C₁₋₆ alkyl (e.g., methyl). In some embodiments, two R^{a} groups, two R^{h} groups, or an R^{a} and an R^{h} are C₁₋₆alkyl (e.g., methyl). In some embodiments, R^{c} is selected from —CH₃, —CH₂CH₂F, —CH₂CHF₂, and —CH₂CH₂CN. In some embodiments, an R^{a} and R^{b} join together to form a 3-6 membered carbocycle, such as a cyclopropane. In some embodiments, an R^{a} and R^{b} attached to the same carbon atom join together to form a 3-6 membered carbocycle, such as a cyclopropane. In some embodiments, an R^{a} and R^{b} join together to form a 3-6 membered heterocycle. In some embodiments, R¹ is selected from:

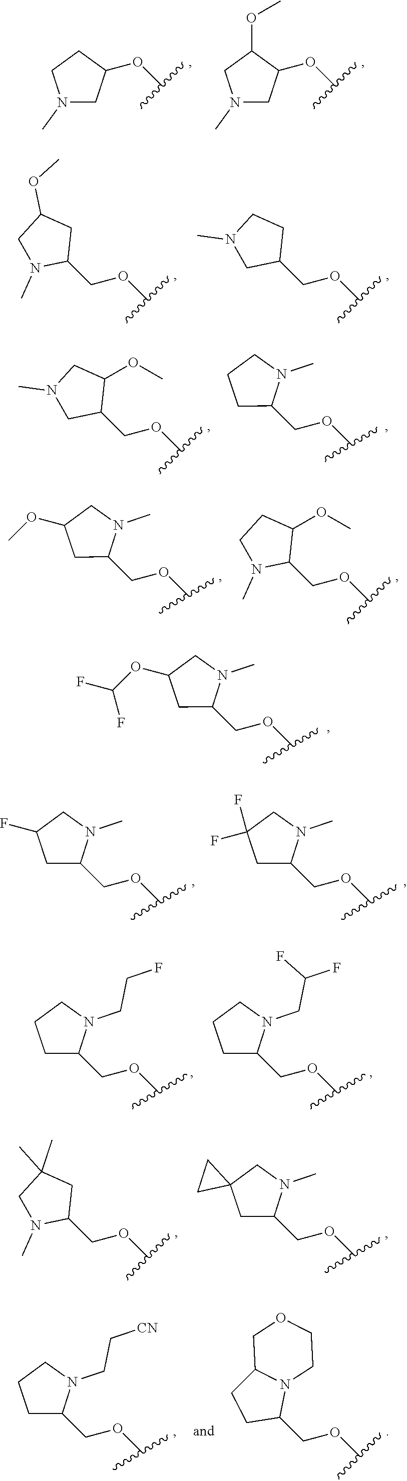

In some embodiments, for a compound of any one of Formulas III, III′, IIIA, IIIA1, IIIB, IIIC, IIIC1, IIID, IIIE, IIIF, IIIG, IIIJ, IIIK, IIIL, IIIK, IIIN, IIIP, IIIQ, IIIR, IIIS, and IIIT, R¹ is selected from:

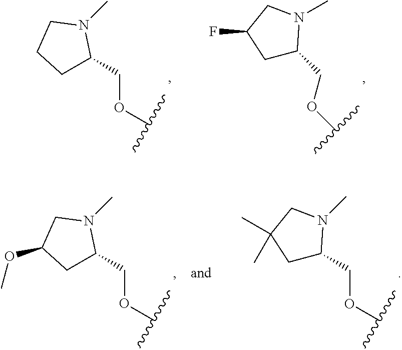

In some embodiments, for a compound of any one of Formulas III, III′, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, and IIIT, R¹ is selected from:

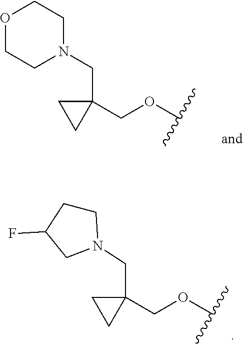 [SMILES: *OCC1(CN2CCOCC2)CC1 ; *OCC1(CN2CCC(F)C2)CC1]

In some embodiments, for a compound of any one of Formulas III, III, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, and IIIT, each R¹¹ is independently selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆akyl that is unsubstituted or substituted with one or more R²⁰. In some embodiments, each R¹¹ is independently selected from —OR¹², =O, =N(R¹⁴), —C(O)(C₁₋₆alkylene)CN, —C(O)(C₁₋₆alkylene)OR¹⁴, —C(O)(C₁₋₆ alkyl), —C(O)O(C₁₋₆ alkyl), —C(O)N(R¹⁴)₂, —C(O)(3-6 membered carbocycle or heterocycle), —S(O)(C₁₋₆alkyl), —N(R¹⁴)C(O)(C₁₋₆alkyl), —N(R¹⁴)C(O)(C₁₋₆alkylene)OR¹⁴, halogen, —CN, a 3-6 membered carbocycle or heterocycle, a 5-6 membered heteroaryl, and C₁₋₆alkyl, wherein any C₁₋₆alkyl, carbocycle, heterocycle, or heteroaryl is unsubstituted or substituted with one or more R². In some embodiments, each R¹¹ is independently selected from —OR¹², —C(O)(C₁₋₆alkylene)CN, —C(O)(C₁₋₆alkylene)OR¹³, —C(O)(C₁₋₆alkyl), —C(O)O(C₁₋₆alkyl), —C(O)N(R¹⁴)₂, —C(O)(3-6 membered carbocycle or heterocycle), —S(O)₂(C₁₋₆alkyl), —N(R¹⁴)C(O)(C₁₋₆alkyl), —N(R¹⁴)C(O)(C₁₋₆alkylene)OR¹⁴, halogen, —CN, a 3-6 membered carbocycle or heterocycle, a 5-6 membered heteroaryl, and C₁₋₆akyl, wherein any C₁₋₆alkyl, carbocycle, heterocycle, or heteroaryl is unsubstituted or substituted with one or more R²⁰.

In some embodiments, for a compound of any one of Formulas III, III′, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, and IIIT, R⁵ is a halogen (e.g., F or Cl). In some embodiments, R⁵ is Cl. In some embodiments, R³ is F. In some embodiments, R⁵ is selected from C₁₋₆alkyl that is unsubstituted or substituted with one or more R¹³. In some embodiments, R⁵ is selected from C₁₋₂alkyl that is unsubstituted or substituted with one or more R¹³. In some embodiments, R⁵ is selected from C₁₋₆alkyl that is unsubstituted, such as methyl or ethyl. In some embodiments, R⁵ is selected from C₁₋₆alkyl that is substituted with one or more halogens or —CN. In some embodiments, R⁵ is C₁₋₆alkyl that is substituted with one or more halogens, such as one or more fluorines. In some embodiments, R⁵ is —CF₃. In some embodiments, R⁵ is —CHF₂. In some embodiments, R⁵ is selected from —CF₂H, —CF₃, —CH₂CN, and —CH₂CH₃. In some embodiments, R⁵ is selected from —CH₃, —CH₂CH₃, —CF₂H, —CF₃, —CF₂CH₃, and —CH₂CN. In some embodiments, R⁵ is C₁₋₆alkyl that is substituted with one or more R¹³, wherein each R¹³ is independently selected from —OR¹⁴, —CN, and —N(R¹⁴)₂. In some embodiments, R⁵ is —CH₂CN.

In some embodiments, for a compound of any one of Formulas III, III′, IIIA, IIIA1, IIIB, IIIC, IIIC1, IIID, IIIE, IIIF, IIIG, IIIH, IIIU, IIIK, IIIL, IIIM, IIIN, IIIP, IIIQ, IIIR, IIIS, and IIIT, R⁷ is Cl. In some embodiments, R⁷ is F.

In some embodiments, for a compound of any one of Formulas Ill, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, and IIIT, the compound is a not a compound included in Table 2, or a salt, ester, tautomer, prodrug, zwitterionic form, or stereoisomer(s) thereof.

TABLE 2

| Compound Number | Structure |
|---|---|
| III-54a | 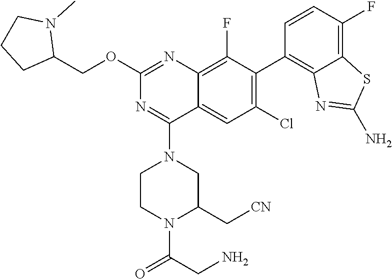 [SMILES: CN1CCCC1COc1nc(N2CCN(C(=O)CN)C(CC#N)C2)c2cc(Cl)c(-c3ccc(F)c4sc(N)nc34)c(F)c2n1] |
| III-202 | 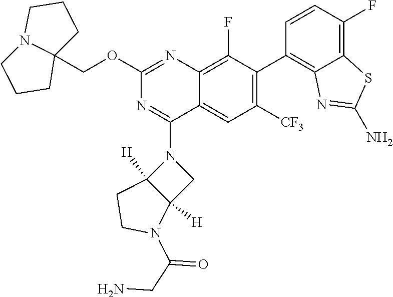 [SMILES: NCC(=O)N1CC[C@@H]2[C@H]1CN2c1nc(OCC23CCCN2CCC3)nc2c(F)c(-c3ccc(F)c4sc(N)nc34)c(C(F)(F)F)cc12] |
| III-104a | 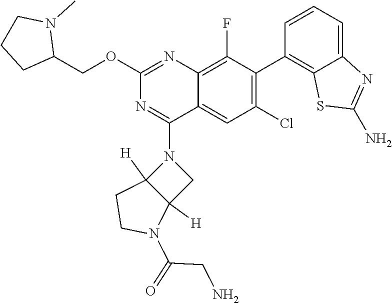 [SMILES: CN1CCCC1COc1nc(N2CC3C2CCN3C(=O)CN)c2cc(Cl)c(-c3cccc4nc(N)sc34)c(F)c2n1] |
| III-22la | 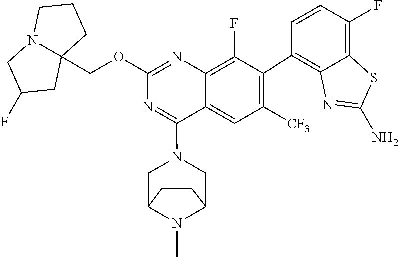 [SMILES: CN1C2CCC1CN(c1nc(OCC34CCCN3CC(F)C4)nc3c(F)c(-c4ccc(F)c5sc(N)nc45)c(C(F)(F)F)cc13)C2] |
| III-108a | 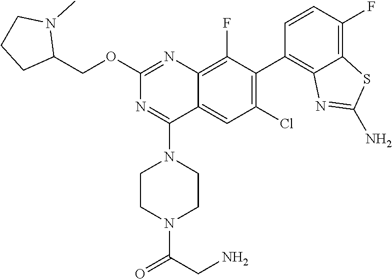 [SMILES: CN1CCCC1COc1nc(N2CCN(C(=O)CN)CC2)c2cc(Cl)c(-c3ccc(F)c4sc(N)nc34)c(F)c2n1] |
| III-272 | 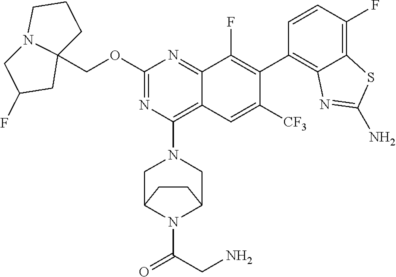 [SMILES: NCC(=O)N1C2CCC1CN(c1nc(OCC34CCCN3CC(F)C4)nc3c(F)c(-c4ccc(F)c5sc(N)nc45)c(C(F)(F)F)cc13)C2] |
| III-116a | 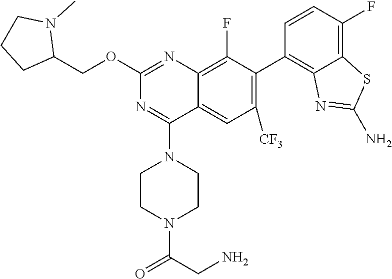 [SMILES: CN1CCCC1COc1nc(N2CCN(C(=O)CN)CC2)c2cc(C(F)(F)F)c(-c3ccc(F)c4sc(N)nc34)c(F)c2n1] |
| III-193 | 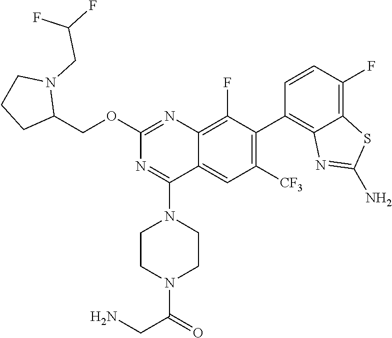 [SMILES: NCC(=O)N1CCN(c2nc(OCC3CCCN3CC(F)F)nc3c(F)c(-c4ccc(F)c5sc(N)nc45)c(C(F)(F)F)cc23)CC1] |
| III-127 | 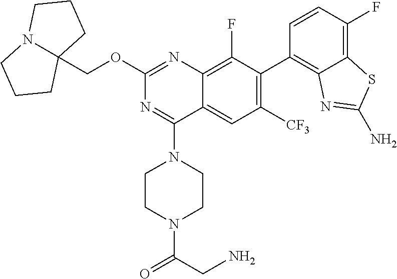 [SMILES: NCC(=O)N1CCN(c2nc(OCC34CCCN3CCC4)nc3c(F)c(-c4ccc(F)c5sc(N)nc45)c(C(F)(F)F)cc23)CC1] |
| III-315 | 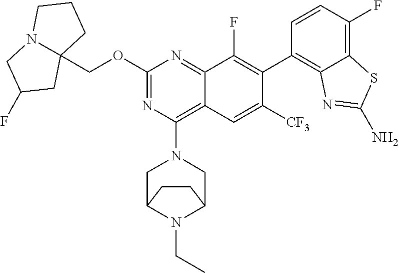 [SMILES: CCN1C2CCC1CN(c1nc(OCC34CCCN3CC(F)C4)nc3c(F)c(-c4ccc(F)c5sc(N)nc45)c(C(F)(F)F)cc13)C2] |
| III-181 | 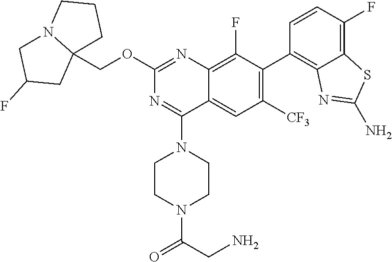 [SMILES: NCC(=O)N1CCN(c2nc(OCC34CCCN3CC(F)C4)nc3c(F)c(-c4ccc(F)c5sc(N)nc45)c(C(F)(F)F)cc23)CC1] |
| III-316 | 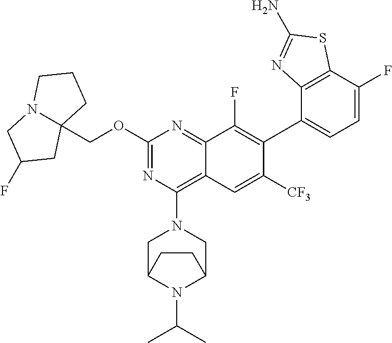 [SMILES: CC(C)N1C2CCC1CN(c1nc(OCC34CCCN3CC(F)C4)nc3c(F)c(-c4ccc(F)c5sc(N)nc45)c(C(F)(F)F)cc13)C2] |
| III-162 | 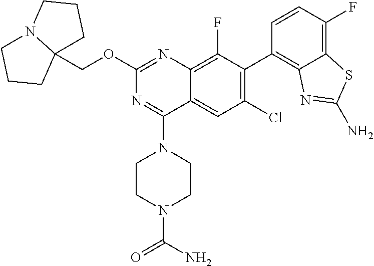 [SMILES: NC(=O)N1CCN(c2nc(OCC34CCCN3CCC4)nc3c(F)c(-c4ccc(F)c5sc(N)nc45)c(Cl)cc23)CC1] |
| III-165 | 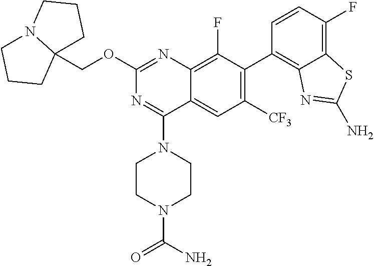 [SMILES: NC(=O)N1CCN(c2nc(OCC34CCCN3CCC4)nc3c(F)c(-c4ccc(F)c5sc(N)nc45)c(C(F)(F)F)cc23)CC1] |
| III-186 | 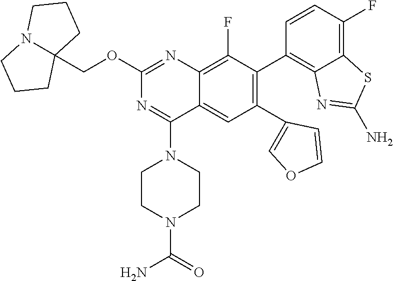 [SMILES: NC(=O)N1CCN(c2nc(OCC34CCCN3CCC4)nc3c(F)c(-c4ccc(F)c5sc(N)nc45)c(-c4ccoc4)cc23)CC1] |
| III-273 | 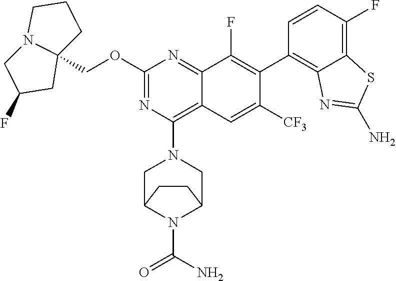 [SMILES: NC(=O)N1C2CCC1CN(c1nc(OC[C@@]34CCCN3C[C@H](F)C4)nc3c(F)c(-c4ccc(F)c5sc(N)nc45)c(C(F)(F)F)cc13)C2] |

In another aspect, the present disclosure provides a compound represented by Formula IV:

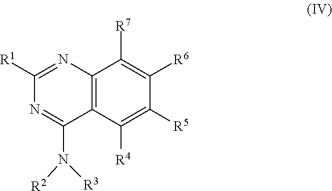

or a salt (e.g., pharmaceutically acceptable salt), ester, tautomer, prodrug, zwitterionic form, or stereoisomer(s) thereof, wherein:
R¹ is selected from —OR⁸ and

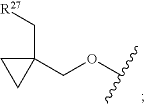

R² is selected from H, C₁₋₆akyl, and a 3-6 membered carbocycle, wherein any C₁₋₆ alkyl is unsubstituted or is substituted with one or more R¹³;
R³ is selected from C₁₋₆alkyl that is unsubstituted or is substituted with one or more R¹⁰;
   R⁴ is H;
   R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
   R⁶ is a bicyclic heteroaryl substituted with one or more R¹⁵;
   R⁷ is selected from halogen, —CN, and H;
   R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
   each R¹⁰ is independently selected from —OR¹⁴, ═O, —CN, —N(R¹⁴)₂, a 3-6 membered carbocycle, a 3-6 membered heterocycle, a 5-6 membered heteroaryl, phenyl, —S(O)₂(C₁₋₆alkyl), —N(R¹⁴)C(O(C₁₋₆alkyl), C₁₋₆alkyl, and halogen, wherein any C₁₋₆alkyl is unsubstituted or substituted with one or more R²⁰, any 3-6 membered carbocycle is unsubstituted or substituted with one or more R², and any 3-6 membered heterocycle or 5-6 membered heteroaryl is unsubstituted or substituted with one or more ═O, R², or R³;
   each R¹² is independently selected from C₁₋₆ alkyl, C₂₋₆ alkenyl, and H, wherein any C₁₋₆alkyl or C₂₋₄ alkenyl is unsubstituted or substituted with one or more R¹⁶;
   each R¹¹ is independently selected from —OR¹⁴, —CN, —N(R¹⁴)₂, and halogen,
   each R¹⁴ is independently selected from C₁₋₆ alkyl, C₂₋₆ alkenyl, and H;
   each R¹⁵ is independently selected from deuterium, halogen, —N(R¹²)₂, —CN, and C₁₋₆alkyl, wherein any C₁₋₆alkyl is unsubstituted or substituted with one or more R¹³;
   each R²⁰ is independently selected from —OH, —OC₁₋₆alkyl, —CN, —NH₂, —NHC₁₋₆akyl, and halogen;
   R⁷ is a 3-6 membered heterocycle including one or more heteroatoms selected from N, O, and S, wherein the heterocycle is unsubstituted or substituted with one or more R²⁸;
   each R²⁸ is independently selected from C₁₋₆alkyl and halogen; and
   R^{a} and R^{b} are each independently selected from halogen, C₁₋₆ alkyl, —OR¹², and H, wherein any C₁₋₆-alkyl is unsubstituted or is substituted with one or more R¹³, and wherein an R^{a} and R^{b} optionally join together to form a 3-6 membered carbocycle or heterocycle.

In some embodiments, the present disclosure provides a compound of Formula IV or a salt (e.g., a pharmaceutically acceptable salt) thereof.

In some embodiments, the present disclosure provides a compound of Formula IV, wherein:
R¹ is selected from —OR⁸;
R² is selected from H, C₁₋₆alkyl, and a 3-6 membered carbocycle, wherein any C₁₋₆ alkyl is unsubstituted or is substituted with one or more R¹³;
R³ is selected from C₁₋₆alkyl that is unsubstituted or is substituted with one or more R¹⁰;
R⁴ is H;
R⁵ is selected from C₁₋₆alkyl, wherein any C₁₋₆alkyl is unsubstituted or substituted with one or more R¹³;
R⁶ is a bicyclic heteroaryl substituted with one or more R¹⁵;
R⁷ is selected from halogen;
R⁸ is selected from an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹⁰ is independently selected from —OR¹⁴, ═O, —CN, —N(R¹⁴)₂, a 3-6 membered carbocycle, a 3-6 membered heterocycle, a 5-6 membered heteroaryl, phenyl, —S(O)₂(C₁₋₆alkyl), —N(R¹⁴)C(O(C₁₋₆alkyl), and halogen, wherein any 3-6 membered carbocycle is unsubstituted or substituted with one or more R², and any 3-6 membered heterocycle or 5-6 membered heteroaryl is unsubstituted or substituted with one or more ═O, R¹², or R¹³;
each R¹² is independently selected from C₁₋₆ alkyl and H, wherein any C₁₋₆alkyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl and H;
each R¹⁵ is independently selected from halogen, —N(R¹⁴)₂, and —CN; and R^{a} and R^{b} are each independently selected from halogen, C₁₋₆ alkyl, —OR¹², and H, wherein an R^{a} and R^{b} optionally join together to form a 3-6 membered carbocycle or heterocycle.

In some embodiments, the present disclosure provides a compound of Formula IV-a:

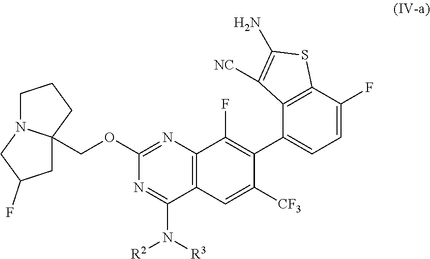

or a salt (e.g., pharmaceutically acceptable salt), ester, tautomer, prodrug, zwitterionic form, or stereoisomer(s) thereof, wherein R² and R³ are as defined for Formula IV above and described in classes and subclasses herein, both singly and in combination. In some embodiments, the present disclosure provides a compound of Formula IV-a, or a salt (e.g., pharmaceutically acceptable salt) thereof.

In some embodiments, the compound is a compound according to Formula IVA:

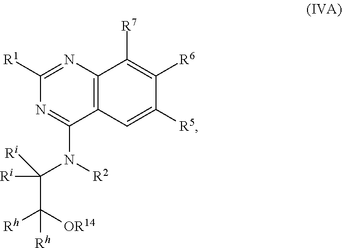

or a salt (e.g., pharmaceutically acceptable salt), ester, tautomer, prodrug, zwitterionic form, or stereoisomer(s) thereof, wherein:
R¹ is selected from —OR⁸ and

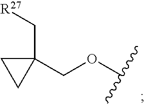

R² is selected from H and C₁₋₆alkyl, wherein any C₁₋₆ alkyl is unsubstituted or is substituted with one or more R¹³;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁶ is a bicyclic heteroaryl substituted with one or more R^{1S};
R⁷ is selected from halogen, —CN, and H;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹⁰ is independently selected from —OR¹⁴, ═O, —CN, —N(R¹⁴)₂, a 3-6 membered carbocycle, C₁₋₆alkyl, and halogen, wherein any C₁₋₆alkyl is unsubstituted or substituted with one or more R²⁰, and wherein any 3-6 membered carbocycle is unsubstituted or substituted with one or more R¹²;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹³;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen;
R^{a} and R^{b} are each independently selected from halogen, C₁₋₆ alkyl, —OR¹², and H, wherein any C₁₋₆alkyl is unsubstituted or is substituted with one or more R¹³, and wherein an R^{a} and R^{b} optionally join together to form a 3-6 membered carbocycle or heterocycle; and
each Rⁱ and R^{h} is independently selected from H and R¹⁰.

In some embodiments, the present disclosure provides a compound of Formula IVA or a salt (e.g., a pharmaceutically acceptable salt) thereof.

In some embodiments, for a compound according to Formula IVA, each Rⁱ and R^{h} is independently selected from H and R¹⁰, and each R¹⁰ is independently selected from —OR¹⁴, ═O, —CN, —NH(C₁₋₆alkyl), —N(C₁₋₆alkyl)₂, a 3-6 membered carbocycle, C₁₋₆alkyl, and halogen, wherein any C₁₋₆alkyl is unsubstituted or substituted with one or more R²⁰, and wherein any 3-6 membered carbocycle is unsubstituted or substituted with one or more R¹². In some embodiments, each Rⁱ and R^{h} is independently selected from H and C₁₋₆alkyl, wherein any C₁₋₆alkyl is unsubstituted or substituted with one or more R²⁰. In some embodiments, each Rⁱ and R^{h} is hydrogen. It will be appreciated that when R^{h} or Rⁱ is —O, the R^{h} or Rⁱ on the same carbon atom is absent, such that the carbon has proper valency.

In some embodiments, for a compound according to Formula IVA, R¹⁴ is H.

In some embodiments, for a compound according to Formula IV or IVA, R⁶ is selected from:

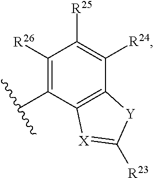

wherein X is selected from N and C—CN; Y is selected from O and S; R²³ is selected from —N(R¹²)₂, C₁₋₆alkyl, and C₁₋₆ alkyl-N(R¹⁴)₂, wherein any C₁₋₆alkyl is unsubstituted or substituted with one or more R¹³; and R²⁴, R²⁵, and R²⁶ are independently selected from H, deuterium, halogen, and C₁₋₆alkyl, wherein any C₁₋₆alkyl is unsubstituted or substituted with one or more R¹³. In some embodiments, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R¹²)₂. In some embodiments, X is C—CN, Y is S, and R²³ is —NH₂. In some embodiments, R²⁴ is halogen (e.g., fluoro). In some embodiments, R²⁶ is deuterium.

In some embodiments, for a compound according to Formula IV or IVA, R⁶ is selected from:

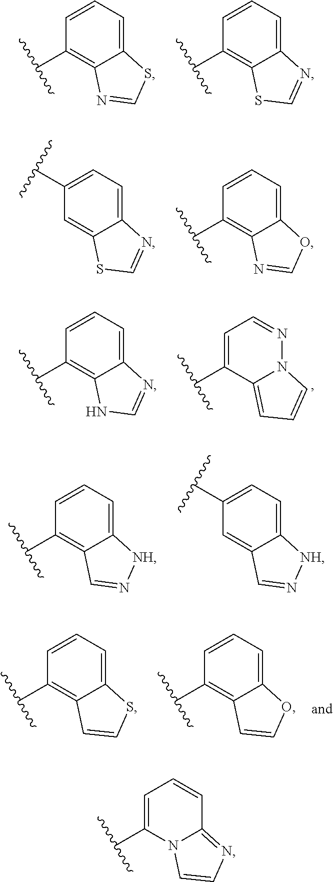

any of which is substituted with one or more R¹⁵.

In some embodiments, for a compound according to Formula IV or IVA, R⁶ is selected from:

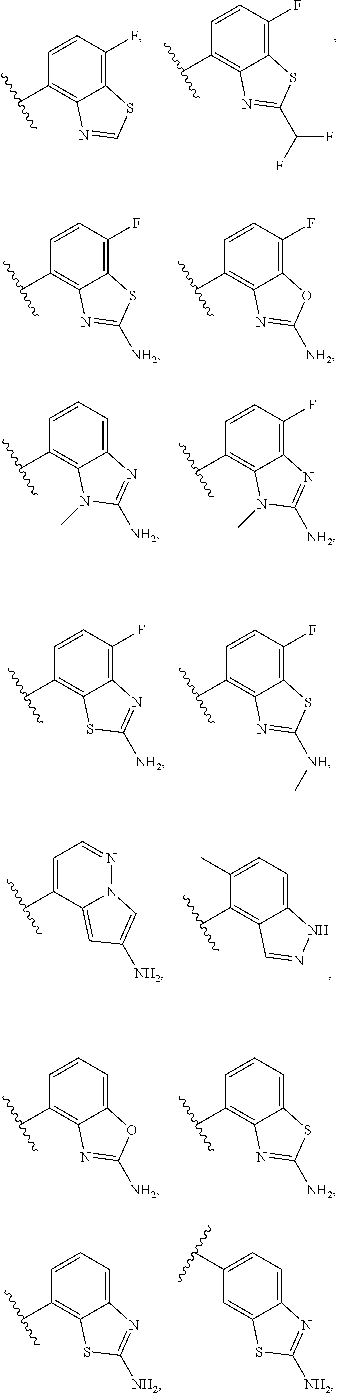

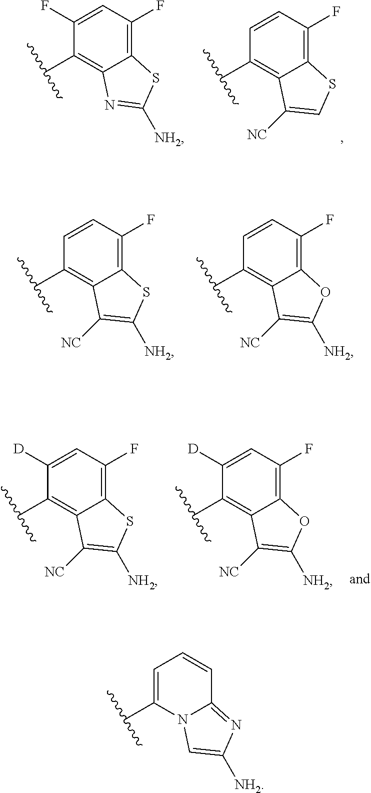

In some embodiments, for a compound according to Formula IV or IVA, R⁶ is selected from:

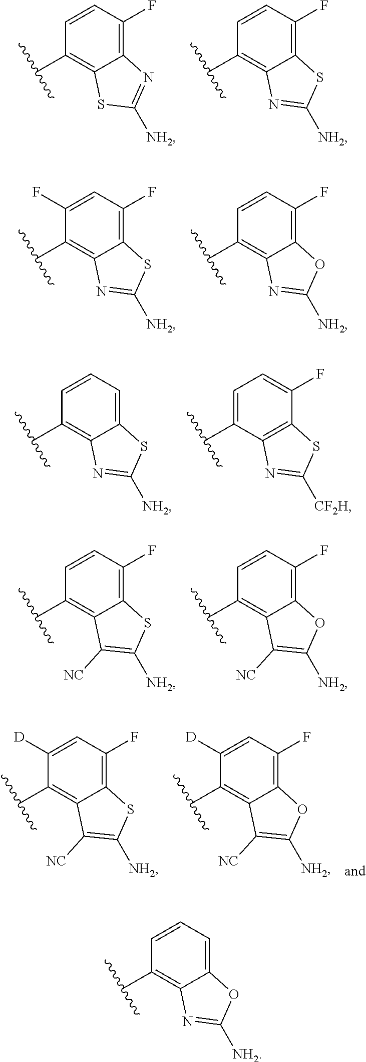

In some embodiments, for a compound according to Formula IV or IVA, R⁶ is

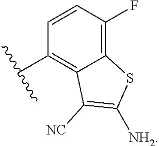 [SMILES: *c1ccc(F)c2sc(N)c(C#N)c12]

In some embodiments, the compound is a compound according to Formula IVB:

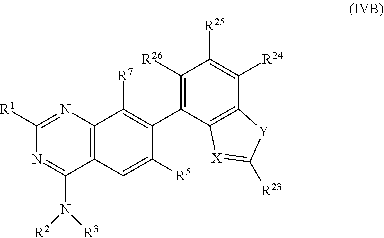

or a salt (e.g., pharmaceutically acceptable salt), ester, tautomer, prodrug, zwitterionic form, or stereoisomer(s) thereof, wherein:
R¹ is selected from —OR⁸ and

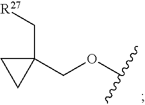

R² is selected from H, C₁₋₆alkyl, and a 3-6 membered carbocycle, wherein any C₁₋₆ alkyl is unsubstituted or is substituted with one or more R¹³;
R³ is selected from C₁₋₆alkyl that is unsubstituted or is substituted with one or more R¹³;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁷ is selected from halogen, —CN, and H;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹⁰ is independently selected from —OR¹⁴, ═O, —CN, —N(R¹⁴)₂, a 3-6 membered carbocycle, a 3-6 membered heterocycle, a 5-6 membered heteroaryl, phenyl, —S(O)₂(C₁₋₆alkyl), —N(R¹⁴)C(O)(C₁₋₆alkyl), C₁₋₆alkyl, and halogen, wherein any C₁₋₆alkyl is unsubstituted or substituted with one or more R², any 3-6 membered carbocycle is unsubstituted or substituted with one or more R², and any 3-6 membered heterocycle or 5-6 membered heteroaryl is unsubstituted or substituted with one or more ═O, R¹², or R¹³;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen;
R^{a} and R^{b} are each independently selected from halogen, C₁₋₆alkyl, —OR¹², and H, wherein any C₁₋₆alkyl is unsubstituted or is substituted with one or more R¹³, and wherein an R^{a} and R^{b} optionally join together to form a 3-6 membered carbocycle or heterocycle;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, deuterium, halogen, —OR²⁸, and C₁₋₆alkyl, wherein any C₁₋₆alkyl is unsubstituted or substituted with one or more R¹³.

In some embodiments, the present disclosure provides a compound of Formula IVB or a salt (e.g., a pharmaceutically acceptable salt) thereof.

In some embodiments, for a compound of any one of Formulas IV, IVA, or IVB, R¹ is selected from —OR⁸, wherein R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl. In some embodiments, R⁸ is a heterocycle or an alkylheterocycle, wherein any heterocycle contains 4-8 members and is substituted with one or more R^{a} or R^{b}. In some embodiments, R⁸ is a heterocycle that is unsubstituted or substituted with one or more R^{a} or R^{b}. In some embodiments, R⁸ is an alkylheterocycle that is unsubstituted or substituted with one or more R^{a} or R^{b}. In some embodiments, R⁸ is —CH₂(heterocycle), where the heterocycle is unsubstituted or substituted with one or more R^{a} or R^{b}. In some embodiments, a heterocycle or a heterocycle of an alkylheterocycle is a 4-6 membered monocyclic heterocycle having 1-2 heteroatoms independently selected from N, O, and S. In some embodiments, a heterocycle or a heterocycle of an alkylheterocycle is an 8-membered bicyclic heterocycle having 1-2 heteroatoms independently selected from N, O, and S. In some embodiments, a heterocycle or a heterocycle of an alkylheterocycle is substituted with one or more R^{a} or R^{b}, wherein the one or more R^{a} or R^{b} is a halogen (e.g., F). In some embodiments, a heterocycle or a heterocycle of an alkylheterocycle is substituted with one or more R^{a} or R^{b}, wherein the one or more R^{a} or R^{h} is a C₁₋₆alkyl (e.g., methyl). In some embodiments, a heterocycle or a heterocycle of an alkylheterocycle is substituted with one or more R^{a} or R^{b}, wherein the one or more R^{a} or R^{b} is a —OR² (e.g., —OCH₃).

In some embodiments, for a compound of any one of Formulas IV, IVA, or IVB, R¹ is selected from:

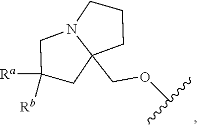

wherein R^{a} and R^{b} are each independently selected from halogen, C₁₋₆alkyl, —OR¹², and H, wherein any C₁₋₆alkyl is unsubstituted or is substituted with one or more R¹³. In some embodiments, R^{a} is a halogen. In some embodiments, R^{a} is F. In some embodiments, R^{a} is C₁₋₆alkyl that is unsubstituted or is substituted with one or more R¹³. In some embodiments, R^{a} is methyl. In some embodiments, R^{a} is —OC₁₋₆alkyl. In some embodiments, R^{a} is H. In some embodiments, R^{b} is H. In some embodiments, R^{a} is a halogen. In some embodiments, R^{b} is F. In some embodiments, R^{b} is C₁₋₆alkyl that is unsubstituted or is substituted with one or more R¹³. In some embodiments, R^{b} is methyl. In some embodiments, each of R^{a} and R^{b} is F. In some embodiments, each of R^{a} and R^{b} is methyl. In some embodiments, R¹ is selected from:

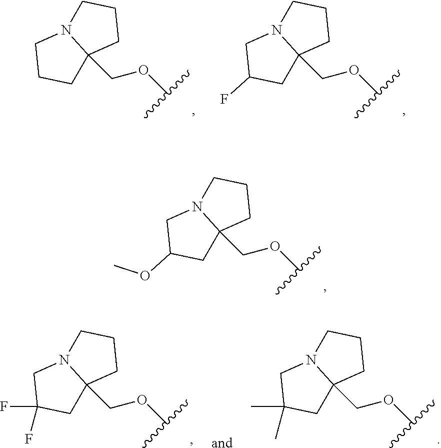 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

In some embodiments, for a compound of any one of Formulas IV, IVA, or IVB, R¹ is selected from:

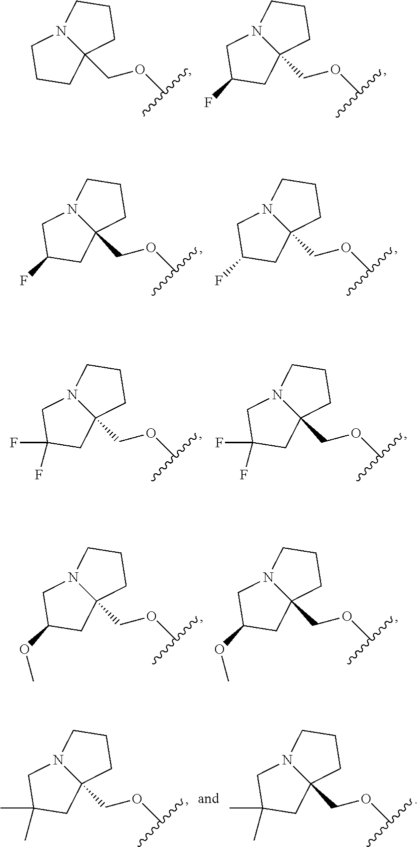

In some embodiments, for a compound of any one of Formulas IV, IVA, or IVB, R¹ is selected from:

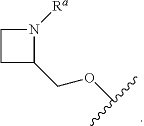

In some embodiments, R^{a} is C₁₋₆alkyl that is unsubstituted or substituted with one or more R¹³. In some embodiments, R^{a} is methyl. In some embodiments, R¹ is selected from:

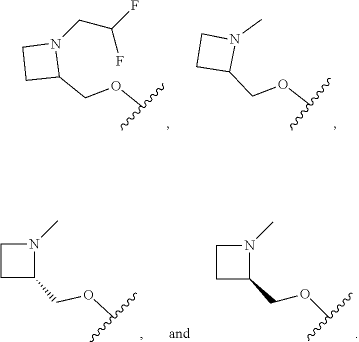

In some embodiments, for a compound of any one of Formulas IV, IVA, or IVB, R¹ is selected from:

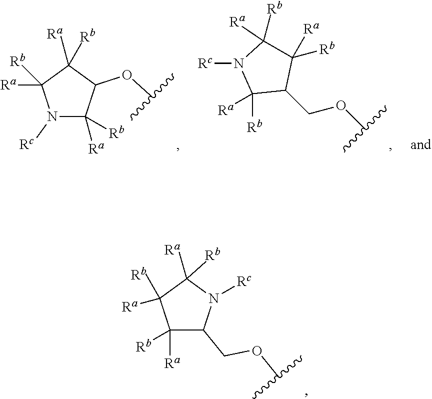

wherein each R^{a} and R^{b} is independently selected from halogen, C₁₋₆ alkyl, —OR¹², and H; and R^{c} is selected from C₁₋₆ alkyl, wherein any C₁₋₆alkyl is unsubstituted or is substituted with one or more R¹³, and wherein an R^{a} and R^{b} or R^{c} optionally join together to form a 3-6 membered carbocycle or heterocycle. In some embodiments, each R^{a} and R^{b} is independently selected from halogen, C₁₋₆ alkyl, —OR¹², and H; and R^{c} is selected from C₁₋₆ alkyl, wherein any C₁₋₆ alkyl is unsubstituted or is substituted with one or more R¹³, and wherein an R^{a} and R^{b} attached to the same carbon atom join together to form a 3-6 membered carbocycle. In some embodiments, each R^{a} and R^{b} is independently selected from halogen, C₁₋₆ alkyl, —OR¹², and H; and wherein an R^{a} and R^{c} join together to form a 3-6 membered heterocycle. In some embodiments, each R^{a} and R^{b} is independently selected from halogen, C₁₋₆ alkyl, —OR¹², and H; and R^{c} is selected from C₁₋₆ alkyl, wherein any C₁₋₆ alkyl is unsubstituted or is substituted with one or more R¹³. In some embodiments, one R^{a} or R^{b} is selected from halogen, C₁₋₆alkyl, and —OR¹², and the other R^{a} and R^{b} groups are H. In some embodiments, one R^{a} or R^{b} is halogen (e.g., F). In some embodiments, two R^{a} groups, two R^{b} groups, or an R^{a} and an R^{b} are halogen (e.g., F). In some embodiments, one R^{a} or R^{b} is —OR¹² (e.g., —OCH₃ or —OCHF₂). In some embodiments, one R^{a} or R^{b} is C₁₋₆ alkyl (e.g., methyl). In some embodiments, two R^{c} groups, two R^{b} groups, or an R^{a} and an R^{b} are C₁₋₆alkyl (e.g., methyl). In some embodiments, R^{c} is selected from —CH₃, —CH₂CH₂F, —CH₂CHF₂, and —CH₂CH₂CN. In some embodiments, an R^{a} and R^{b} join together to form a 3-6 membered carbocycle, such as a cyclopropane. In some embodiments, an R^{a} and R^{b} attached to the same carbon atom join together to form a 3-6 membered carbocycle, such as a cyclopropane. In some embodiments, an R^{a} and R^{c} join together to form a 3-6 membered heterocycle. In some embodiments, R¹ is selected from:

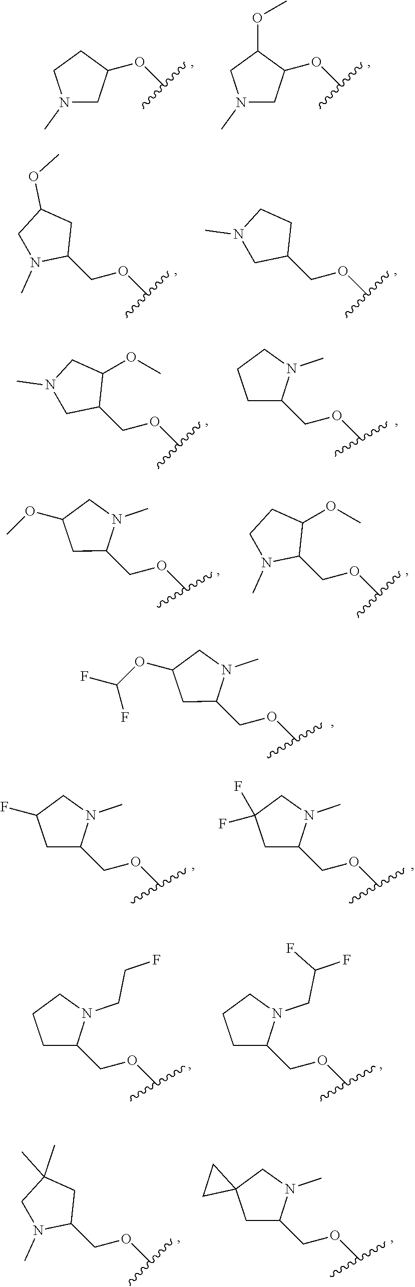

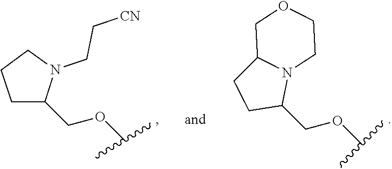 [SMILES: *OCC1CCCN1CCC#N ; *OCC1CCC2COCCN12]

In some embodiments, for a compound of any one of Formulas IV, IVA, or IVB, R¹ is selected from:

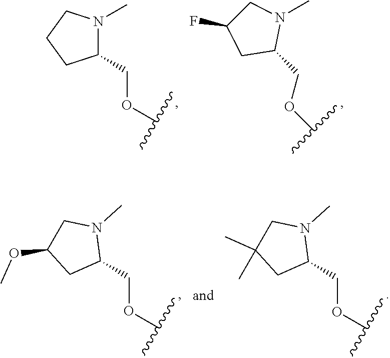

In some embodiments, for a compound of any one of Formulas IV, IVA, or IVB, R¹ is selected from:

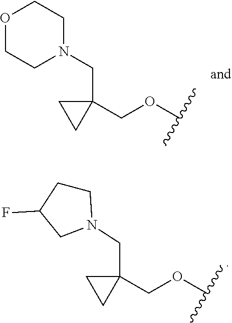 [SMILES: *OCC1(CN2CCOCC2)CC1 ; *OCC1(CN2CCC(F)C2)CC1]

In some embodiments, the compound is a compound according to Formula IVC,

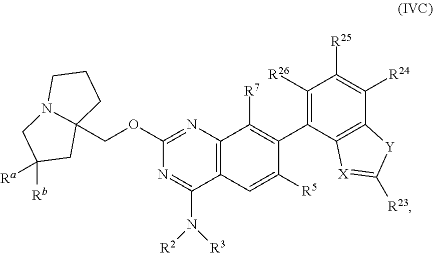

or a salt (e.g., pharmaceutically acceptable salt), ester, tautomer, prodrug, zwitterionic form, or stereoisomer(s) thereof, wherein:
R² is selected from H, C₁₋₆alkyl, and a 3-6 membered carbocycle, wherein any C₁₋₆ alkyl is unsubstituted or is substituted with one or more R¹³;
R³ is selected from C₁₋₆alkyl that is unsubstituted or is substituted with one or more R¹⁰;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁷ is selected from halogen, —CN, and H;
each R¹⁰ is independently selected from —OR¹⁴, ═O, —CN, —N(R¹⁴)₂, a 3-6 membered carbocycle, a 3-6 membered heterocycle, a 5-6 membered heteroaryl, phenyl, S(O)₂(C₁₋₆alkyl), —N(R¹⁴)C(O)(C₁₋₆alkyl), C₁₋₆alkyl, and halogen, wherein any C₁₋₆-alkyl is unsubstituted or substituted with one or more R²⁰, and wherein any 3-6 membered carbocycle is unsubstituted or substituted with one or more R², and any 3-6 heterocycle or 5-6 membered heteroaryl is unsubstituted or substituted with one or more ═O, R¹², or R¹³;
each R¹² is independently selected from C₁₋₆alkyl, C₂a alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R²⁰ is independently selected from —OH, —OC-alkyl, —CN, —NH₂, —NHC₁₋₆alkyl, and halogen;
R^{a} and R^{b} are each independently selected from halogen, C₁₋₆ alkyl, —OR¹², and H, wherein any C₁₋₆alkyl is unsubstituted or is substituted with one or more R¹³;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, deuterium, halogen, —OR¹², and C₁₋₆alkyl, wherein any C₁₋₆alkyl is unsubstituted or substituted with one or more R¹³.

In some embodiments, the present disclosure provides a compound of Formula IVC or a salt (e.g., a pharmaceutically acceptable salt) thereof.

In some embodiments, for a compound according to Formula IVC, R^{a} is a halogen. In some embodiments, R^{a} is F. In some embodiments, R^{a} is C₁₋₆alkyl that is unsubstituted or is substituted with one or more R¹³. In some embodiments, R^{a} is methyl. In some embodiments, R^{a} is —OC₁₋₆alkyl. In some embodiments, R^{a} is H. In some embodiments, R^{b} is H. In some embodiments, R^{b} is a halogen. In some embodiments, R^{b} is F. In some embodiments, R^{b} is C₁₋₆alkyl that is unsubstituted or is substituted with one or more R¹³. In some embodiments, R^{b} is methyl. In some embodiments, each of R^{a} and R^{b} is F. In some embodiments, each of R^{a} and R^{b} is methyl.

In some embodiments, for a compound according to Formula IVB or IVC, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R¹²)₂. In some embodiments, X is C—CN, Y is S, and R²³ is —NH₂. In some embodiments, X is C—CN, Y is S, R²³ is —N(R¹²)₂, and R²⁴ is a halogen (e.g., F). In some embodiments, X is C—CN, Y is S, R¹ is —N(R¹²)₂, and one or more of R²⁴, R²⁵, and R²⁶ is a halogen (e.g., F). In some embodiments, R²⁶ is deuterium.

In some embodiments, for a compound according to any one of Formulas IV, IV-a, IVA, IVB, or IVC, R² is H. In some embodiments, R² is selected from C₁₋₆alkyl that is unsubstituted or is substituted with one or more R¹³. In some embodiments, R² is selected from C₁₋₆alkyl that is unsubstituted. In some embodiments, R² is selected from C₁₋₆alkyl that is substituted with one or more R¹³. In some embodiments, R² is selected from C₁₋₂alkyl that is unsubstituted or is substituted with one or more R¹³. In some embodiments, R² is selected from C₁₋₂alkyl that is unsubstituted. In some embodiments, R² is selected from C₁₋₂alkyl that is substituted with one or more R¹³. In some embodiments, R² is methyl. In some embodiments, R² is ethyl. In some embodiments, R² is a 3-6 membered carbocycle (e.g., a cyclopropane).

In some embodiments, for a compound according to any one of Formula IV, IV-a, IVB, and IVC, R³ is selected from C₁₋₆alkyl that is unsubstituted. In some embodiments, R³ is selected from C₁₋₆alkyl that is substituted with one or more R¹³. In some embodiments, R³ is selected from C₁₋₆alkyl that is substituted with one or more R¹⁰, wherein each R¹⁰ is independently selected from —OR¹⁴, ═O, —CN, —N(R¹⁴)₂, a 3-6 membered carbocycle, a 5-6 membered heteroaryl, —S(O)₂(C₁₋₆alkyl), —N(R¹²)C(O)(C₁₋₆alkyl), C₁₋₆alkyl, and halogen, wherein any C₁₋₆alkyl is unsubstituted or substituted with one or more R²⁰, any 3-6 membered carbocycle is unsubstituted or substituted with one or more R¹², and any 5-6 membered heteroaryl is unsubstituted or substituted with one or more R¹³. In some embodiments, R³ is selected from C₁₋₆alkyl that is substituted with one or more R¹⁰, wherein each R¹⁰ is independently selected from —OR¹⁴, —O, —CN, —NH(R¹⁶), —N(R¹⁶)₂, a 3-6 membered carbocycle, C₁₋₆alkyl, and halogen, wherein any C₁₋₆alkyl is unsubstituted or substituted with one or more R²⁰, wherein any 3-6 membered carbocycle is unsubstituted or substituted with one or more R², and wherein each R¹⁶ is independently selected from C₁₋₆alkyl and C₂₋₆alkenyl. In some embodiments, R³ is selected from C₁₋₃alkyl that is unsubstituted or is substituted with one or more R¹⁰. In some embodiments, R³ is selected from C₁₋₃alkyl that is unsubstituted or is substituted with one or more R¹⁰, wherein each R¹⁰ is independently selected from —OR¹⁴, ═O, —CN, —N(R¹⁴)₂, a 3-6 membered carbocycle, a 5-6 membered heteroaryl, —S(O)₂(C₁₋₆alkyl). —N(R¹⁴)C(O)(C₁₋₆alkyl), C₁₋₆alkyl, and halogen, wherein any C₁₋₆alkyl is unsubstituted or substituted with one or more R²⁰, any 3-6 membered carbocycle is unsubstituted or substituted with one or more R¹², and any 5-6 membered heteroaryl is unsubstituted or substituted with one or more R¹³. In some embodiments, R³ is selected from C₁₋₃alkyl that is unsubstituted or is substituted with one or more R¹⁰, wherein each R¹⁰ is independently selected from —OR¹⁴, ═O, —CN, —NH(R¹⁶), —N(R¹⁶)₂, a 3-6 membered carbocycle, C₁₋₆alkyl, and halogen, wherein any C₁₋₆alkyl is unsubstituted or substituted with one or more R²⁰, wherein any 3-6 membered carbocycle is unsubstituted or substituted with one or more R¹², and wherein each R¹⁶ is independently selected from C₁₋₆alkyl and C₂₋₆alkenyl. In some embodiments, R³ is selected from C₁₋₆alkyl that is unsubstituted. In some embodiments, R³ is selected from C₁₋₃alkyl that is substituted with one or more R¹⁰. In some embodiments, R³ is selected from C₁₋₃alkyl that is substituted with one or more R¹⁰, wherein each R¹⁶ is independently selected from —OR¹⁴, ═O, —CN, —N(R¹⁴)₂, a 3-6 membered carbocycle, a 5-6 membered heteroaryl, —S(O)₂(C₁₋₆alkyl), —N(R¹⁴)C(O)(C₁₋₆alkyl), C₁₋₆alkyl, and halogen, wherein any C₁₋₆alkyl is unsubstituted or substituted with one or more R²⁰, any 3-6 membered carbocycle is unsubstituted or substituted with one or more R¹³, and any 5-6 membered heteroaryl is unsubstituted or substituted with one or more R¹³. In some embodiments, R³ is selected from C₁₋₃alkyl that is substituted with one or more R¹⁰, wherein each R¹⁰ is independently selected from —OR¹⁴, ═O, —CN, —NH(R¹⁶), —N(R¹⁴)₂, a 3-6 membered carbocycle, C₁₋₆alkyl, and halogen, wherein any C₁₋₆alkyl is unsubstituted or substituted with one or more R¹³, wherein any 3-6 membered carbocycle is unsubstituted or substituted with one or more R¹², and wherein each R¹⁶ is independently selected from C₁₋₆alkyl and C₂₋₆alkenyl. In some embodiments, R³ is C₂₋₃alkyl that is unsubstituted or is substituted with one or more R¹⁰. In some embodiments, R³ is C₂₋₃alkyl that is unsubstituted or is substituted with one or more R¹⁰, wherein each R¹⁰ is independently selected from —OR¹⁴, ═O, —CN, —N(R¹⁴)₂, a 3-6 membered carbocycle, a 5-6 membered heteroaryl, —S(O)₂(C₁₋₆alkyl), —N(R¹⁴)C(O)(C₁₋₆alkyl), C₁₋₆alkyl, and halogen, wherein any C₁₋₆alkyl is unsubstituted or substituted with one or more R, any 3-6 membered carbocycle is unsubstituted or substituted with one or more R², and any 5-6 membered heteraryl is unsubstituted or substituted with one or more R¹³. In some embodiments, R³ is C₂₋₃alkyl that is unsubstituted or is substituted with one or more R¹⁰, wherein each R¹⁰ is independently selected from —OR¹⁴, ═O, —CN, —NH(R¹⁶), —N(R¹⁶)₂, a 3-6 membered carbocycle, C₁₋₆alkyl, and halogen, wherein any C₁₋₆alkyl is unsubstituted or substituted with one or more R²⁰, wherein any 3-6 membered carbocycle is unsubstituted or substituted with one or more R¹², and wherein each R¹⁶ is independently selected from C₁₋₆alkyl and C₂₋₆alkenyl. In some embodiments, R³ is C₂₋₃alkyl that is unsubstituted. In some embodiments, R³ is C₂₋₃alkyl that is substituted with one or more R¹⁰. In some embodiments, R³ is C₂₋₃alkyl that is substituted with one or more R¹⁰, wherein each R¹⁶ is independently selected from —OR¹⁴, ═O, —CN, —NH(R¹⁶), —N(R¹⁶)₂, a 3-6 membered carbocycle, C₁₋₆alkyl, and halogen, wherein any C₁₋₆alkyl is unsubstituted or substituted with one or more R²⁰, wherein any 3-6 membered carbocycle is unsubstituted or substituted with one or more R¹², and wherein each R¹⁶ is independently selected from C₁₋₆alkyl and C₂₋₆ alkenyl. In some embodiments, R³ is C₂₋₃alkyl that is substituted with one or more R¹³, wherein the one or more R¹⁰ are selected from —OR¹⁴, —CN, C₁₋₆alkyl, and halogen, wherein any C₁₋₆-alkyl is unsubstituted or substituted with one or more R²⁰. In some embodiments, R³ is C₂₋₃alkyl that is substituted with one or more R¹⁰, wherein the one or more R¹⁰ are selected from —OR¹⁴ and C₁₋₆alkyl, wherein any C₁₋₆-alkyl is unsubstituted or substituted with one or more R²⁰. In some embodiments, R³ is C₂₋₆alkyl that is substituted with one or more R¹⁰, wherein the one or more R¹⁰ are halogen. In some embodiments, R³ is C₂₋₃alkyl that is substituted with one or more F. In some embodiments, R³ is C₁₋₃alkyl that is substituted with a 5-6 membered heteroaryl that is unsubstituted or substituted with one or more ═O, R¹², or R¹³. In some embodiments, R³ is C₂₋₆ alkyl that is substituted with a 5-6 membered heteroaryl that is unsubstituted or substituted with one or more R¹³. In some embodiments, R³ is C₁₋₃alkyl that is substituted with an oxazole, isoxazole, pyridine, pyrazine, pyridazine, or pyrimidine that is unsubstituted or substituted with one or more ═O, R², or R³. In some embodiments, R³ is C₂₋₃alkyl that is substituted with a pyridine that is unsubstituted or substituted with one or more R¹³. In some embodiments, R³ is C₂₋₆ alkyl that is substituted with a pyridine that is substituted with one or more R¹³. In some embodiments, R³ is C₂₋₃alkyl that is substituted with —N(R¹⁴)C(O)(C₁₋₆alkyl). In some embodiments, R¹ is C₂₋₃alkyl that is substituted with —S(O)₂(C₁₋₆alkyl). In some embodiments, R³ is C₁₋₃alkyl that is substituted with a 3-6 membered heterocycle (e.g., oxetane). In some embodiments, R³ is C₁₋₃alkyl that is substituted with a phenyl.

In some embodiments, for a compound according to any one of Formula IV, IV-a, IVB and IVC, the moiety

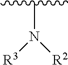

is selected from:

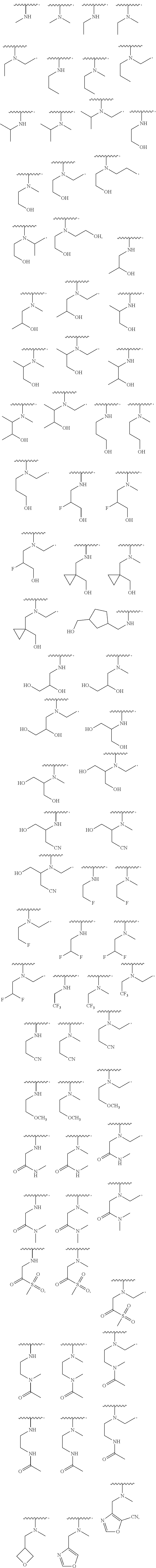

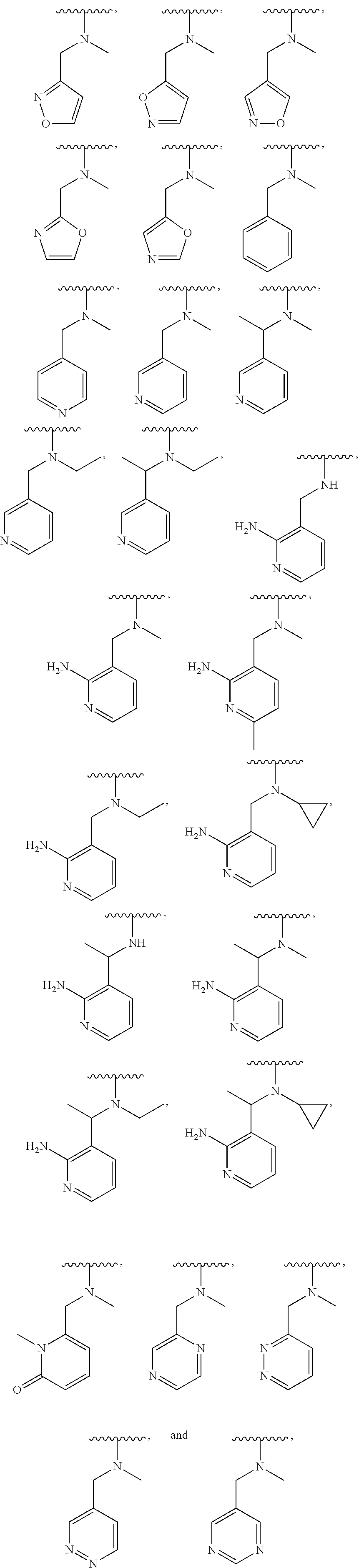

any of which is optionally further substituted with one or more R¹⁰.

In some embodiments, for a compound according to any one of Formula IV, TVA, IVB, and VC, R⁵ is H. In some embodiments, R⁵ is a halogen (e.g., F or Cl). In some embodiments, R⁵ is Cl. In some embodiments, R⁵ is F. In some embodiments, R⁵ is —CN. In some embodiments, R⁵ is —OR¹², wherein R¹² is selected from C₁₋₆ alkyl and H. In some embodiments, R⁵ is —OCH₃. In some embodiments, R⁵ is —OCF₃.

In some embodiments, R⁵ is selected from C₁₋₆alkyl that is unsubstituted or substituted with one or more R¹³. In some embodiments, R⁵ is selected from C₁₋₂alkyl that is unsubstituted or substituted with one or more R¹³. In some embodiments, R⁵ is selected from C₁₋₆alkyl that is unsubstituted, such as methyl or ethyl. In some embodiments, R⁵ is selected from C₁₋₆alkyl that is substituted with one or more halogens or —CN. In some embodiments, R⁵ is C₁₋₆alkyl that is substituted with one or more halogens, such as one or more fluorines. In some embodiments, R⁵ is —CF₃. In some embodiments, R⁵ is —CHF₂. In some embodiments, R⁵ is selected from —CF₂H, —CF₃, —CH₂CN, and —CH₂CH₃. In some embodiments, R⁵ is selected from —CH₃, —CH₂CH₃, —CF₂H, —CF₃, —CF₂CH₃, and —CH₂CN. In some embodiments, R⁵ is C₁₋₆alkyl that is substituted with one or more R¹³, wherein each R¹³ is independently selected from —OR¹⁴, —CN, and —N(R¹⁴)₂. In some embodiments, R⁵ is —CH₂CN. In some embodiments, R⁵ is a 3-6 membered heterocycle. In some embodiments, R⁵ is a 5-6 membered heteroaryl, such as a furan.

In some embodiments, for a compound according to any one of Formula IV, IVA, IVB, and IVC, R⁷ is H. In some embodiments, R⁷ is a halogen (e.g., F or Cl). In some embodiments, R⁷ is Cl. In some embodiments, R⁷ is F. In some embodiments, R⁷ is —CN.

In some embodiments, for a compound of any one of Formulas IV, IVA, IVB, and IVC, the compound is a not a compound included in Table 3, or a salt, ester, tautomer, prodrug, zwitterionic form, or stereoisomer(s) thereof.

TABLE 3

| Compound | |
|---|---|
| Number | Structure |
| IV-70a | 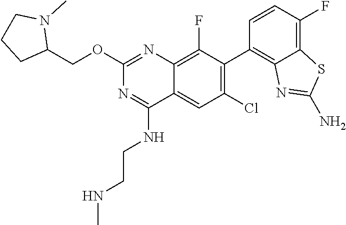 [SMILES: CNCCNc1nc(OCC2CCCN2C)nc2c(F)c(-c3ccc(F)c4sc(N)nc34)c(Cl)cc12] |
| IV-75a | 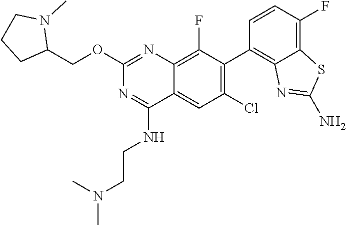 [SMILES: CN(C)CCNc1nc(OCC2CCCN2C)nc2c(F)c(-c3ccc(F)c4sc(N)nc34)c(Cl)cc12] |
| IV-76a | 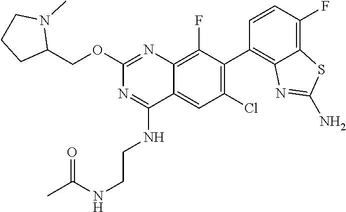 [SMILES: CC(=O)NCCNc1nc(OCC2CCCN2C)nc2c(F)c(-c3ccc(F)c4sc(N)nc34)c(Cl)cc12] |

In another aspect, the present disclosure provides a compound represented by Formula V:

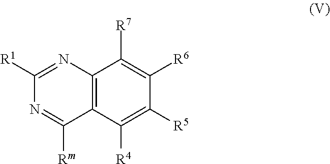

or a salt (e.g., pharmaceutically acceptable salt), ester, tautomer, prodrug, zwitterionic form, or stereoisomer(s) thereof, wherein:
R¹ is selected from —OR⁸

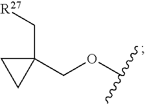

R^{m} is selected from hydrogen and C₁₋₆alkyl, wherein any C₁₋₆alkyl is unsubstituted or substituted with one or more R¹³;
R⁴ is H;
R⁵ is selected from halogen and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆akyl;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹³;
R²⁷ is a 3-6 membered heterocycle including one or more heteroatoms selected from N, O, and S, wherein the heterocycle is unsubstituted or substituted with one or more R¹³;
each R²⁸ is independently selected from C₁₋₆alkyl and halogen; and
R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.

In some embodiments, the present disclosure provides a compound of Formula V or a salt (e.g., a pharmaceutically acceptable salt) thereof.

In some embodiments, the present disclosure provides a compound of Formula V, wherein:
R¹ is selected from —OR⁸;
R^{m} is selected from hydrogen and C₁₋₆alkyl;
R⁴ is H;
R⁵ is C₁₋₆alkyl, wherein any C₁₋₆alkyl is unsubstituted or substituted with one or more R¹³;
R⁶ is a bicyclic heteroaryl, wherein the heteroaryl is substituted with one or more R¹⁵;
R⁷ is selected from halogen:
R⁸ is an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹² is H;
each R¹³ is halogen;
each R¹⁵ is independently selected from halogen, —N(R¹²)₂, and —CN; and
R^{a} and R^{b} are each independently selected from halogen, and H.

In some embodiments, the present disclosure provides a compound of Formula V-a:

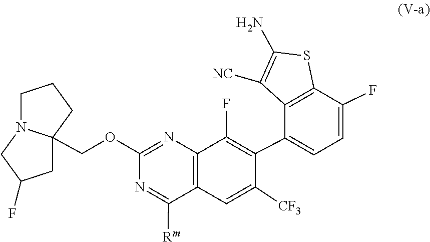

or a salt (e.g., pharmaceutically acceptable salt), ester, tautomer, prodrug, zwitterionic form, or stereoisomer(s) thereof, wherein R^{m} is as defined for Formula V above and described in classes and subclasses herein, both singly and in combination. In some embodiments, the present disclosure provides a compound of Formula V-a, or a salt (e.g., pharmaceutically acceptable salt) thereof.

In some embodiments, for a compound according to Formula V or V-a, R¹ is hydrogen. In some embodiments, R^{m} is C₁₋₆alkyl that is unsubstituted or substituted with one or more R¹³. In some embodiments, R^{m} is C₁₋₆alkyl that is unsubstituted. In some embodiments, R^{m} is methyl that is unsubstituted. In some embodiments, R^{m} is C₁₋₆alkyl that is substituted with one or more R¹³.

In some embodiments, the compound is a compound according to Formula VA:

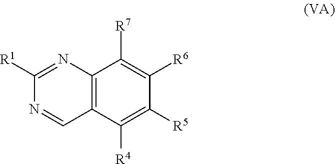

or a salt (e.g., pharmaceutically acceptable salt), ester, tautomer, prodrug, zwitterionic form, or stereoisomer(s) thereof, wherein:
R¹ is selected from —OR⁸ and

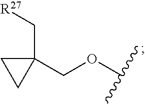

R⁴ is H;
R⁵ is selected from halogen and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₄ alkenyl, and H;
each R¹ is independently selected from deuterium, halogen, —N(R¹²)₂, —CN, and C₁₋₆alkyl, wherein any C₁₋₆alkyl is unsubstituted or substituted with one or more R¹³;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen; and
R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.

In some embodiments, the present disclosure provides a compound of Formula VA or a salt (e.g., a pharmaceutically acceptable salt) thereof.

In some embodiments, the compound is a compound according to Formula VB:

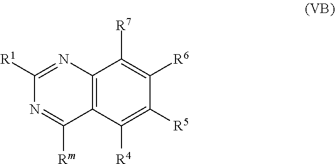

or a salt (e.g., pharmaceutically acceptable salt), ester, tautomer, prodrug, zwitterionic form, or stereoisomer(s) thereof, wherein:
R¹ is selected from —OR⁸ and

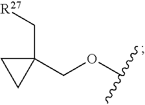

R⁴ is H;
R⁵ is selected from halogen and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆akyl;
each R¹² is independently selected from C₁₋₆ alkyl, C₂₋₆ alkenyl, and H, wherein any C₁₋₆akyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹⁴)₂, —CN, and C₁₋₆alkyl, wherein any C₁₋₆alkyl is unsubstituted or substituted with one or more R¹³;
R²⁷ is a 3-6 membered heterocycle including one or more heteroatoms selected from N, O, and S, wherein the heterocycle is unsubstituted or substituted with one or more R¹³;
each R²⁸ is independently selected from C₁₋₆alkyl and halogen; and
R^{a} and R^{b} are each independently selected from deuterium, halogen, C₁₋₆alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.

In some embodiments, the present disclosure provides a compound of Formula VB or a salt (e.g., a pharmaceutically acceptable salt) thereof.

In some embodiments, for a compound according to any one of Formulas V, VA, and VB, R⁶ is a bicyclic heteroaryl that is substituted with one or more R¹⁵. In some embodiments, R⁶ is selected from:

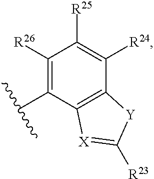

wherein X is selected from N and C—CN; Y is selected from O and S; R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and R²⁴, R²⁵, and R²⁶ are independently selected from H, deuterium, halogen, and C₁₋₆alkyl, wherein any C₁₋₆alkyl is unsubstituted or substituted with one or more R¹³. In some embodiments, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R¹²)₂. In some embodiments, X is C—CN, Y is S, and R²³ is —NH₂. In some embodiments, R²⁴ is halogen (e.g., fluoro). In some embodiments, R²⁶ is deuterium.

In some embodiments, for a compound according to any one of Formulas V, VA, and VB, R⁶ is selected from:

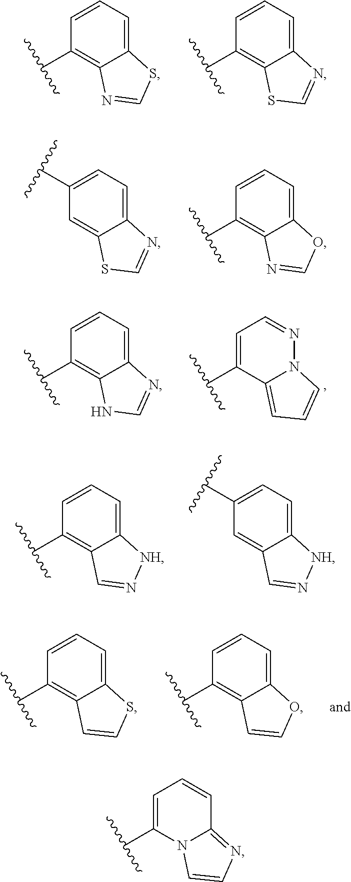

any of which is substituted with one or more R¹⁵.

In some embodiments, for a compound according to any one of Formulas V, VA, and VB, R⁶ is selected from:

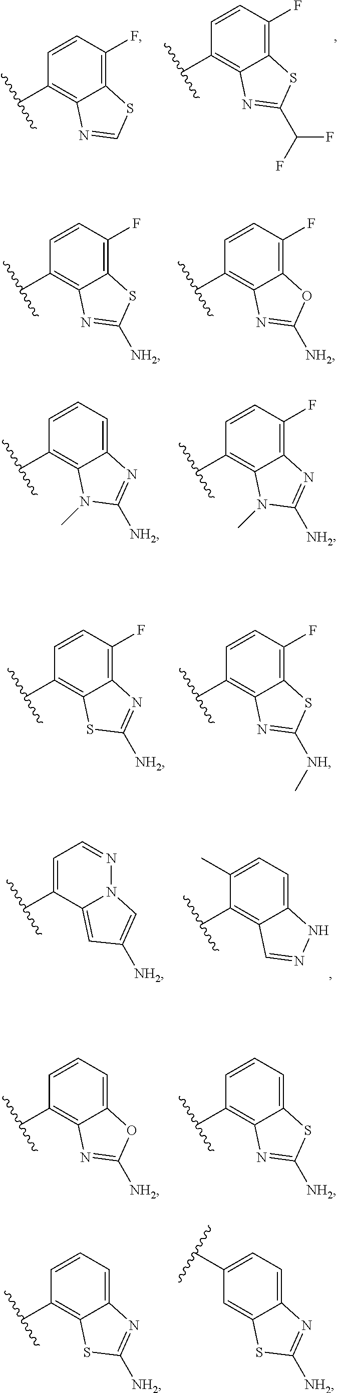

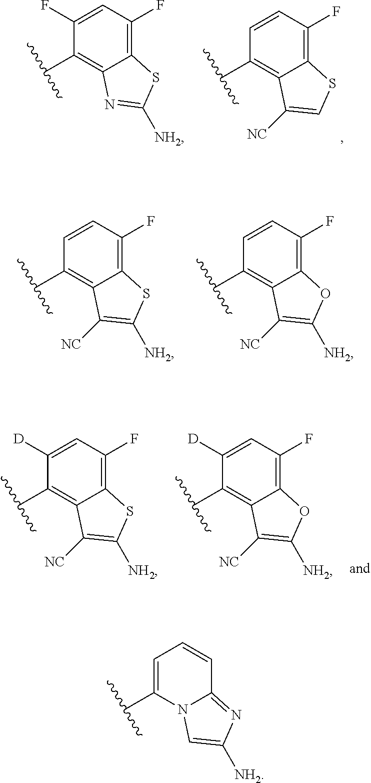

In some embodiments, for a compound according to any one of Formulas V, VA, and VB, R⁶ is selected from:

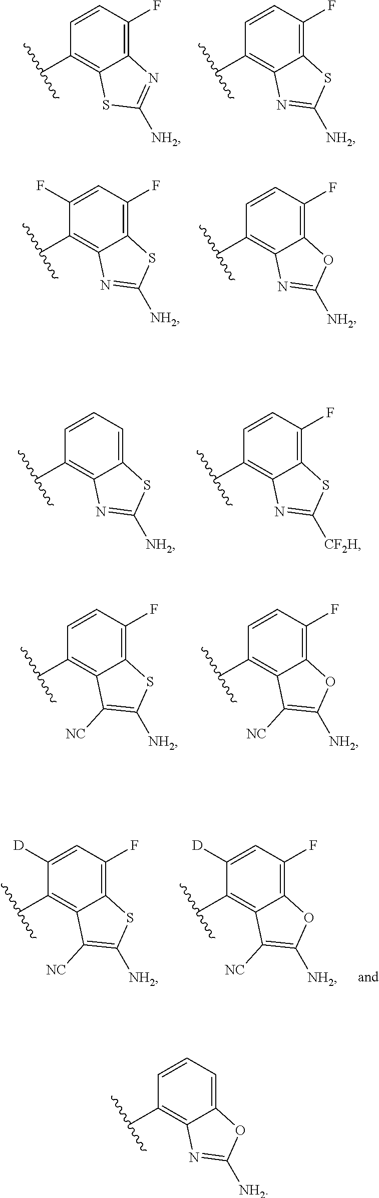

In some embodiments, for a compound according to any one of Formulas V, VA, and VB, R⁶ is

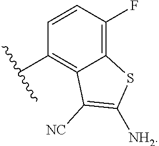 [SMILES: *c1ccc(F)c2sc(N)c(C#N)c12]

In some embodiments, for a compound according to any one of Formulas V, VA, and VB, R⁶ is a monocyclic heteroaryl that is substituted with one or more R¹⁵. In some embodiments, R⁶ is a pyridine substituted with one or more R¹⁵. In some embodiments, at least one R¹⁵ is selected from C₁₋₆alkyl, wherein any C₁₋₆alkyl is unsubstituted or substituted with one or more R¹³. In some embodiments, at least one R¹⁵ is —N(R¹²)₂. In some embodiments, R⁶ has a structure selected from

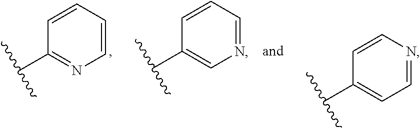 [SMILES: *c1ccccn1 ; *c1cccnc1 ; "and" ; *c1ccncc1]

any of which is optionally substituted with one or more R¹⁵. In some embodiments, R⁶ has the structure

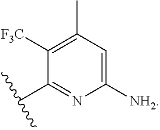 [SMILES: *c1nc(N)cc(C)c1C(F)(F)F]

In some embodiments, the compound is a compound according to Formula VC:

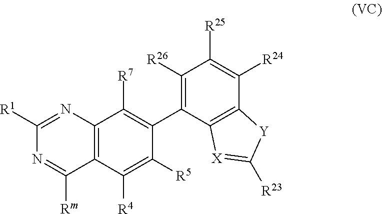

or a salt (e.g., pharmaceutically acceptable salt), ester, tautomer, prodrug, zwitterionic form, or stereoisomer(s) thereof wherein:
R¹ is selected from —OR⁸ and

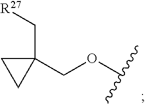

R^{m} is selected from hydrogen and C₁₋₆alkyl, wherein any C₁₋₆alkyl is unsubstituted or substituted with one or more R¹³;
R⁴ is H;
R⁵ is selected from halogen and C₁₋₆alkyl, wherein any C₁₋₄alkyl is unsubstituted or substituted with one or more R¹³;
R⁷ is selected from halogen:
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹³;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen;
R^{a} and R^{b} are each independently selected from deuterium, halogen, C₁₋₆ alkyl, a 3-6 membered carbocycle, —OR¹², and H, wherein an R^{a} and R^{c} optionally join together to form a 3-6 membered carbocycle or heterocycle, and wherein any C₁₋₆alkyl or 3-6 membered carbocycle or heterocycle is unsubstituted or is substituted with one or more R¹³;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, deuterium, halogen, —OR¹², and C₁₋₄alkyl, wherein any C₁₋₆alkyl is unsubstituted or substituted with one or more R¹³.

In some embodiments, the present disclosure provides a compound of Formula VC or a salt (e.g., a pharmaceutically acceptable salt) thereof.

In some embodiments, for a compound of Formula VC, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R¹²)₂. In some embodiments, X is C—CN, Y is S, and R²³ is —NH₂. In some embodiments, X is C—CN, Y is S, R²³ is —N(R¹²)₂, and R²⁴ is a halogen (e.g., F). In some embodiments, X is C—CN, Y is S, R²³ is —N(R¹²)₂, and one or more of R²⁴, R²⁵, and R²⁶ is a halogen (e.g., F). In some embodiments, R²⁶ is deuterium.

In some embodiments, for a compound of any one of Formulas V, VA, VB, and VC, R¹ is selected from —OR⁸, wherein R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆ alkyl. In some embodiments, R⁸ is a heterocycle or an alkylheterocycle, wherein any heterocycle contains 4-8 members and is substituted with one or more R^{a} or R^{b}. In some embodiments, R⁸ is a heterocycle that is unsubstituted or substituted with one or more R^{a} or R^{b}. In some embodiments, R⁸ is an alkylheterocycle that is unsubstituted or substituted with one or more R^{a} or R^{b}. In some embodiments, R⁸ is —CH₂(heterocycle), where the heterocycle is unsubstituted or substituted with one or more R^{a} or R^{b}. In some embodiments, a heterocycle or a heterocycle of an alkylheterocycle is a 4-6 membered monocyclic heterocycle having 1-2 heteroatoms independently selected from N, O, and S. In some embodiments, a heterocycle or a heterocycle of an alkylheterocycle is an 8-membered bicyclic heterocycle having 1-2 heteroatoms independently selected from N, O, and S. In some embodiments, a heterocycle or a heterocycle of an alkylheterocycle is substituted with one or more R^{a} or R^{b}, wherein the one or more R^{a} or R^{b} is a halogen (e.g., F). In some embodiments, a heterocycle or a heterocycle of an alkylheterocycle is substituted with one or more R^{a} or R^{b}, wherein the one or more R^{a} or R^{b} is a C₁₋₆alkyl (e.g., methyl). In some embodiments, a heterocycle or a heterocycle of an alkylheterocycle is substituted with one or more R^{a} or R^{b}, wherein the one or more R^{a} or R^{b} is a —OR¹² (e.g., —OCH₃). In some embodiments, a heterocycle or a heterocycle of an alkylheterocycle is substituted with one or more R^{a} or R^{b}, wherein the one or more R^{a} or R^{b} is a 3-6 membered carbocycle (e.g., a cyclopropane). In some embodiments, a heterocycle or a heterocycle of an alkylheterocycle is substituted with one or more R^{a} or R^{b}, wherein the one or more R^{a} or R^{b} is deuterium.

In some embodiments, for a compound of anyone of Formulas V, VA, VB, and VC, R¹ is selected from:

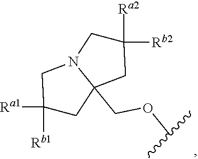

wherein R^{a1}, R^{a2}, R^{b1}, and R^{b} are each independently selected from deuterium, halogen, C₁₋₆alkyl, —OR¹², and H, wherein R^{a2} and R^{b2} can optionally join together to form a 3-6 membered carbocycle, and wherein any C₁₋₆alkyl or 3-6 membered carbocycle is unsubstituted or is substituted with one or more R¹³. In some embodiments, R^{a1} and/or R^{a2} is a halogen. In some embodiments, R^{a1} and/or R^{a2} is F. In some embodiments, R^{a1} and/or R^{a2} is C₁₋₆alkyl that is unsubstituted or is substituted with one or more R^{D}. In some embodiments, R^{a1} and/or R^{a2} is methyl. In some embodiments, R^{a1} and/or R^{a2} is —OC₁₋₄alkyl. In some embodiments, R^{a1} and/or R¹² is H. In some embodiments, R^{b1} and/or R^{b2} is H. In some embodiments, R^{b1} and/or R^{b2} is a halogen. In some embodiments, R^{b1} and/or R^{b2} is F. In some embodiments, R^{b1} and/or R^{b2} is C₁₋₆ alkyl that is unsubstituted or is substituted with one or more R¹³. In some embodiments, R^{b1} and/or R^{b2} is methyl. In some embodiments, each of R^{a1} and R^{b1} is F. In some embodiments, R^{a2} and/or R^{b2 is D}. In some embodiments, R^{a2} and R^{b2} join together to form a 3-6 membered carbocycle (e.g., cyclopropane), which carbocycle is optionally substituted with one or more R¹³. In some embodiments, R^{a1} and R^{b1} join together to form a 3-6 membered carbocycle (e.g., cyclopropane). In some embodiments, R¹ is selected from:

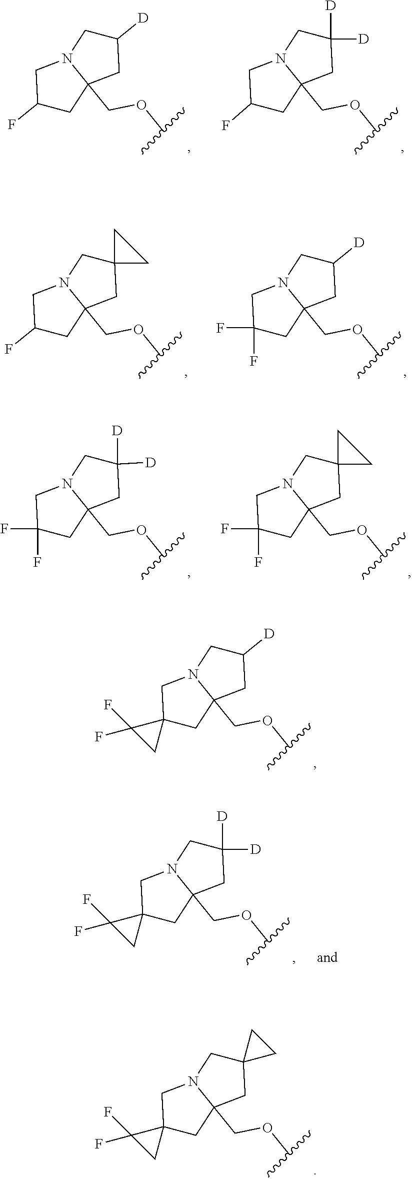

In some embodiments, for a compound of anyone of Formulas V, VA, VB, and VC, R¹ is selected from:

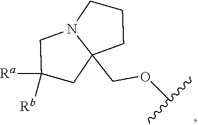

wherein R^{a} and R^{b} are each independently selected from halogen, C₁₋₆alkyl, —OR¹², and H, wherein any C₁₋₆alkyl is unsubstituted or is substituted with one or more R¹³. In some embodiments, R^{a} is a halogen. In some embodiments, R^{a} is F. In some embodiments, R^{a} is C₁₋₆alkyl that is unsubstituted or is substituted with one or more R¹³. In some embodiments, R^{a} is methyl. In some embodiments, R^{a} is —OC₁₋₆alkyl. In some embodiments, R^{a} is H. In some embodiments, R^{b} is H. In some embodiments, R^{b} is a halogen. In some embodiments, R^{b} is F. In some embodiments, R^{b} is C₁₋₆alkyl that is unsubstituted or is substituted with one or more R¹³. In some embodiments, R^{b} is methyl. In some embodiments, each of R^{a} and R^{b} is F. In some embodiments, each of R^{a} and R^{b} is methyl. In some embodiments, R¹ is selected from:

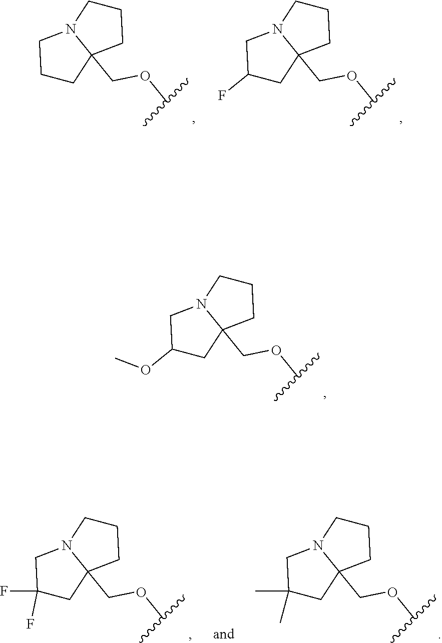 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

In some embodiments, for a compound of any one of Formulas V, VA, VB, and VC, R¹ is selected from:

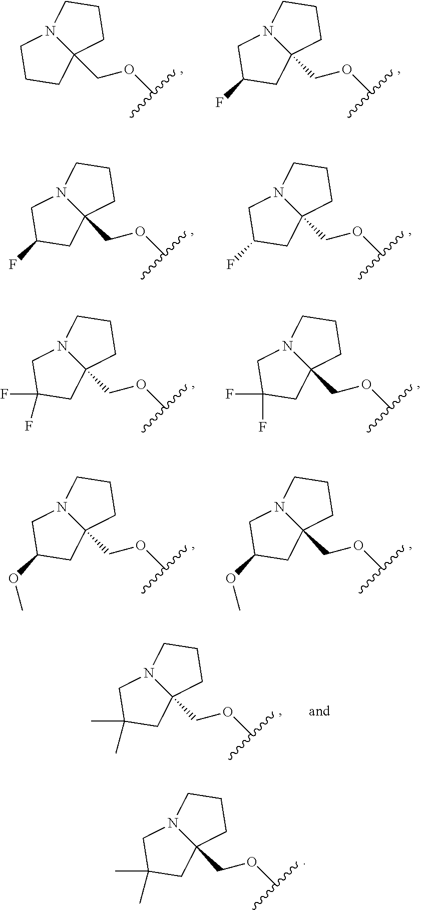

In some embodiments, for a compound of any one of Formulas V, VA, VB, and VC, R¹ is selected from:

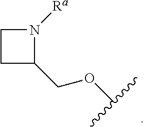

In some embodiments, R^{a} is C₁₋₆alkyl that is unsubstituted or substituted with one or more R¹³. In some embodiments, R^{a} is methyl. In some embodiments, R¹ is selected from:

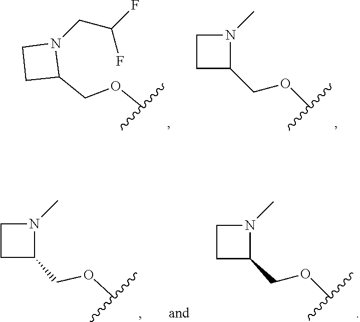

In some embodiments, for a compound of anyone of Formulas V, VA, VB, and VC, R¹ is selected from:

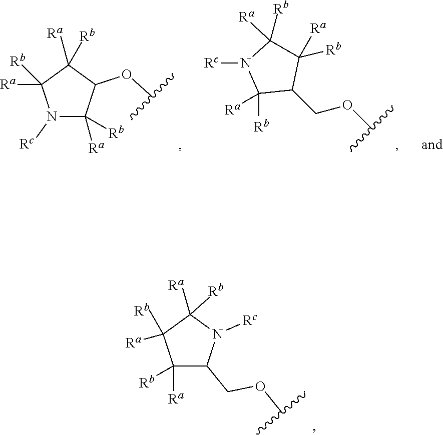

wherein each R^{a} and R^{b} is independently selected from halogen, C₁₋₆alkyl, —OR¹², and H; and R¹ is selected from C₁₋₆ alkyl, wherein an R^{a} and R^{b} or R^{c} optionally join together to form a 3-6 membered carbocycle or heterocycle, and wherein any C₁₋₆ alkyl or 3-6 membered carbocycle or heterocycle is unsubstituted or is substituted with one or more R¹³. In some embodiments, each R^{a} and R^{b} is independently selected from halogen, C₁₋₆ alkyl, —OR¹², and H; and R^{c} is selected from C₁₋₆ alkyl, wherein any C₁₋₄ alkyl is unsubstituted or is substituted with one or more R¹³, and wherein an R^{a} and R^{b} attached to the same carbon atom join together to form a 3-6 membered carbocycle. In some embodiments, each R^{a} and R^{b} is independently selected from halogen, C₁₋₆ alkyl, —OR¹², and H; and wherein an R^{a} and R^{c} join together to form a 3-6 membered heterocycle. In some embodiments, each R^{a} and R^{b} is independently selected from halogen, C₁₋₆ alkyl, —OR¹², and H; and R^{c} is selected from C₁₋₆ alkyl, wherein any C₁₋₆ alkyl is unsubstituted or is substituted with one or more R¹³. In some embodiments, one R^{a} or R^{b} is selected from halogen, C₁₋₆ alkyl, and —OR¹², and the other R^{a} and R^{b} groups are H. In some embodiments, one R^{a} or R^{b} is halogen (e.g., F).

In some embodiments, two R¹ groups, two R^{b} groups, or an R¹ and an R^{b} are halogen (e.g., F). In some embodiments, one R^{a} or R^{b} is —OR¹² (e.g., —OCH₃ or —OCHF₂). In some embodiments, one R^{a} or R^{b} is C₁₋₆ alkyl (e.g., methyl). In some embodiments, two R^{a} groups, two R^{b} groups, or an R¹ and an R^{b} are C₁₋₆ alkyl (e.g., methyl). In some embodiments, R⁵ is selected from —CH₃, —CH₂CH₂F, —CH₂CHF₂, and —CH₂CH₂CN.

In some embodiments, an R^{a} and R^{b} join together to form a 3-6 membered carbocycle, such as a cyclopropane. In some embodiments, an R^{a} and R^{b} attached to the same carbon atom join together to form a 3-6 membered carbocycle, such as a cyclopropane. In some embodiments, an R^{a} and R^{c} join together to form a 3-6 membered heterocycle. In some embodiments, R¹ is selected from:

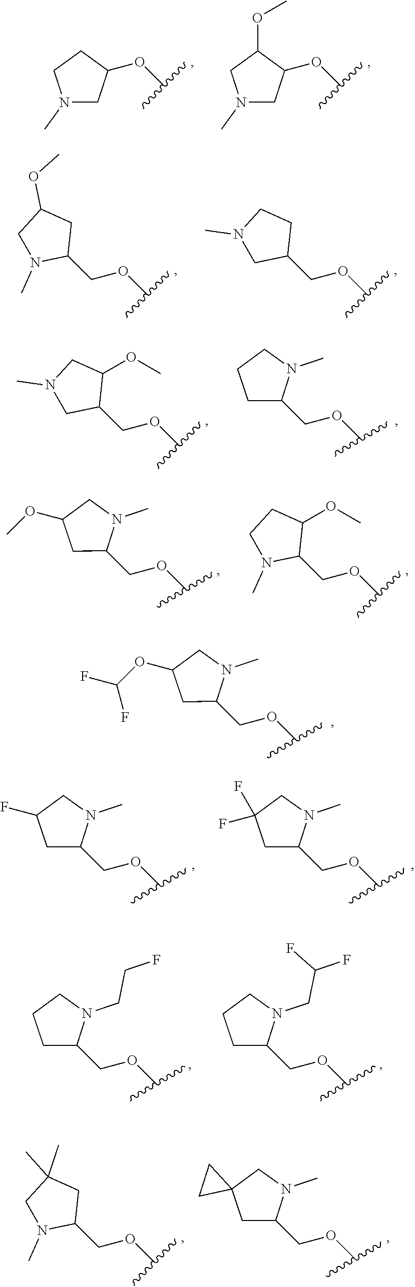

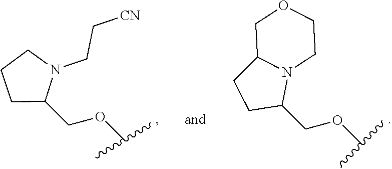 [SMILES: *OCC1CCCN1CCC#N ; *OCC1CCC2COCCN12]

In some embodiments, for a compound of anyone of Formulas V, VA, VB, and VC, R⁵ is selected from:

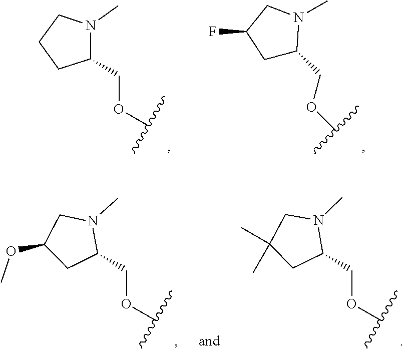

In some embodiments, for a compound of anyone of Formulas V, VA, VB, and VC, R⁵ is selected from:

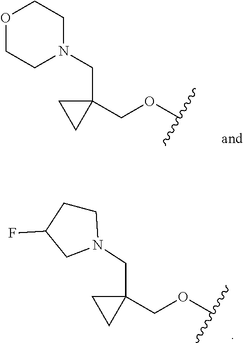 [SMILES: *OCC1(CN2CCOCC2)CC1 ; *OCC1(CN2CCC(F)C2)CC1]

In some embodiments, for a compound of anyone of Formulas V, VA, VB, and VC, R⁵ is a halogen (e.g., F or Cl). In some embodiments, R⁵ is Cl. In some embodiments, R⁵ is F. In some embodiments, R⁵ is selected from C₁₋₆alkyl that is unsubstituted or substituted with one or more R¹³. In some embodiments, R⁵ is selected from C₁₋₂alkyl that is unsubstituted or substituted with one or more R¹³. In some embodiments, R⁵ is selected from C₁₋₆-alkyl that is unsubstituted, such as methyl or ethyl. In some embodiments, R⁵ is selected from C₁₋₆alkyl that is substituted with one or more halogens or —CN. In some embodiments, R⁵ is C₁₋₆alkyl that is substituted with one or more halogens, such as one or more fluorines. In some embodiments, R⁵ is —CF₃. In some embodiments, R⁵ is —CHF₂. In some embodiments, R⁵ is selected from —CF₂H, —CF₃, —CH₂CN, and —CH₂CH₃. In some embodiments, R⁵ is selected from —CH₃, —CH₂CH₃, —CF₂H, —CF₃, —CF₂CH₃, and —CH₂CN. In some embodiments, R⁵ is C₁₋₆alkyl that is substituted with one or more R³, wherein each R¹³ is independently selected from —OR¹⁴, —CN, and —N(R¹⁴)₂. In some embodiments, R⁵ is —CH₂CN.

In some embodiments, for a compound of any one of Formulas V, VA, VB, and VC, R⁷ is Cl. In some embodiments, R⁷ is F.

In another aspect, the present disclosure provides a compound represented by Formula VI:

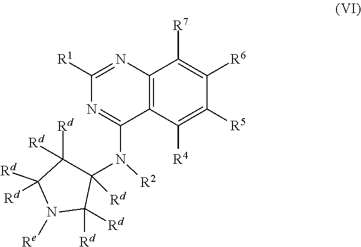

or a salt (e.g., pharmaceutically acceptable salt), ester, tautomer, prodrug, zwitterionic form, or stereoisomer thereof, wherein:
   R¹ is selected from —OR⁸ and

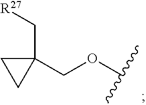

R² is selected from H, C₁₋₆alkyl, and a 3-6 membered carbocycle, wherein any C₁₋₆ alkyl is unsubstituted or is substituted with one or more R¹³;
R⁴ is H;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from H and halogen,
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹² is independently selected from —OR¹⁴, —CN, —N(R¹⁴)₂, and halogen;
each R¹³ is independently selected from a 3-6 membered carbocycle, a 3-6 membered heterocycle, C₁₋₆ alkyl, C₂₋₆ alkenyl, and H, wherein any C₁₋₆alkyl is optionally deuterated;
each R¹⁵ is independently selected from deuterium, halogen, —N(R¹⁴)₂, —CN, and C₁₋₆alkyl, wherein any C₁₋₆alkyl is unsubstituted or substituted with one or more R¹³;
each R²⁰ is independently selected from —OH, —OC₁₋₆alkyl, ═O, —CN, —NH₂, —NHC₁₋₆alkyl, —C(O)(C₁₋₆alkyl), a 3-6 membered carbocycle, a 5-6 membered aryl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆-alkyl and halogen;
R^{a} and R^{b} are each independently selected from deuterium, halogen, C₁₋₆ alkyl, —OR¹², a 3-6 membered carbocycle, and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R³;
each R^{d} is independently selected from H, halogen, and C₁₋₆alkyl, wherein any C₁₋₆alkyl is unsubstituted or substituted with one or more R²⁰;
R^{c} is selected from

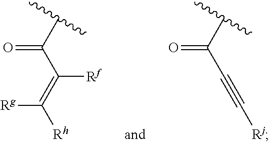

R^{f} is selected from H, halogen, and C₁₋₆alkyl;
R^{g} and R^{h} are independently selected from H and C₁₋₆alkyl; and
R^{j} is selected from C₁₋₆alkyl,
provided that R^{e} is not

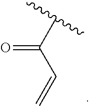 [SMILES: *C(=O)C=C]

In some embodiments, the present disclosure provides a compound of Formula VI, or a salt (e.g., a pharmaceutically acceptable salt) thereof.

In some embodiments, the present disclosure provides a compound of Formula VI, wherein:
R¹ is selected from —OR⁸;
R² is C₁₋₆ alkyl;
R⁴ is H;
R⁵ is C₁₋₆alkyl, wherein any C₁₋₆alkyl is unsubstituted or substituted with one or more R¹³;
R⁶ is a bicyclic heteroaryl, wherein the heteroaryl is substituted with one or more R¹⁵;
R⁷ is halogen;
R⁸ is an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹² is H;
each R¹³ is halogen;
each R¹⁵ is independently selected from halogen, —N(R¹²)₂, and —CN; each R²⁰ is —OC₁₋₆alkyl;
R^{a} and R^{b} are each independently selected from halogen and H;
each R^{d} is independently selected from H and C₁₋₆alkyl, wherein any C₁₋₆alkyl is unsubstituted or substituted with one or more R²⁰;
R^{e} is selected from

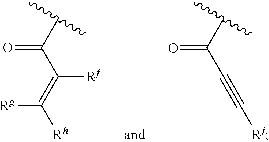

R^{f} is selected from H, halogen, and C₁₋₆alkyl;
R^{g} and R^{h} are independently selected from H and C₁₋₆alkyl; and
R^{j} is selected from C₁₋₆alkyl,
provided that R^{e} is not

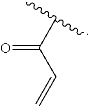 [SMILES: *C(=O)C=C]

In some embodiments, the present disclosure provides a compound of Formula VI-a:

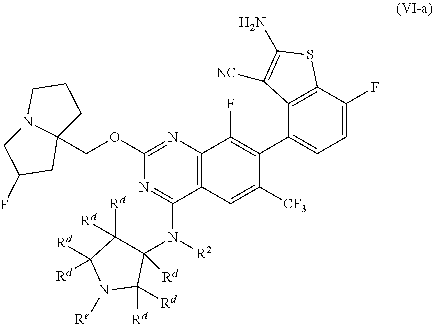

or a salt (e.g., pharmaceutically acceptable salt), ester, tautomer, prodrug, zwitterionic form, or stereoisomer(s) thereof, wherein R², R^{d}, and R^{e} are as defined for Formula VI above and described in classes and subclasses herein, both singly and in combination. In some embodiments, the present disclosure provides a compound of Formula VI-a, or a salt (e.g., pharmaceutically acceptable salt) thereof.

In some embodiments, for a compound according to Formula VI or VI-a, R^{c} is selected from

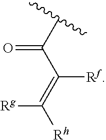

In some embodiments, R^{g} and R^{h} are H, and R^{f} is not H. In some embodiments, R^{g} is H, and at least one of R^{h} and R^{f} is not H. In some embodiments, R^{f} is H, and at least one of R^{g} and R^{h} is not H.

In some embodiments, for a compound according to Formula VI or VI-a, R^{e} is

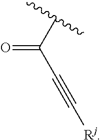

In some embodiments, R^{j} is C₁₋₃ alkyl.

In some embodiments, for a compound according to Formula VI or VI-a, R^{e} is

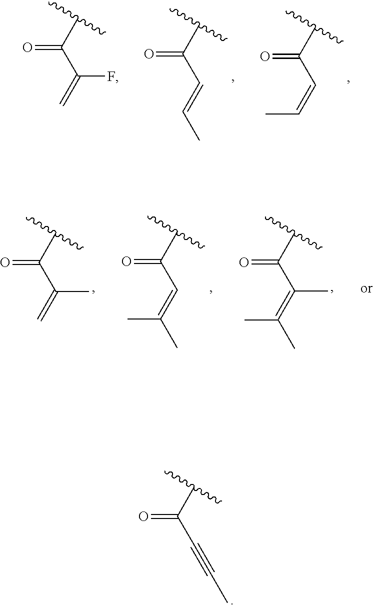

In some embodiments, for a compound according to Formula VI or VI-a, each R^{d} is H. In some embodiments, at least one R^{d} is not H. In some embodiments, at least one R^{d} is selected from C₁₋₆alkyl that is substituted with one or more R²⁰ (e.g., one or more —OC₁₋₆alkyl). In some embodiments, at least one R^{d} is selected from C₁₋₆alkyl. In some embodiments, one R^{d} is selected from C₁₋₆alkyl and each other R^{d} is H.

In some embodiments, for a compound according to Formula VI, R⁶ is a monocyclic heteroaryl that is substituted with one or more R¹⁵. In some embodiments, R⁶ is a pyridine substituted with one or more R¹⁵. In some embodiments, at least one R¹⁵ is selected from C₁₋₆alkyl, wherein any C₁₋₆alkyl is unsubstituted or substituted with one or more R¹³. In some embodiments, at least one R¹⁵ is —N(R¹²)₂. In some embodiments, R⁶ is selected from

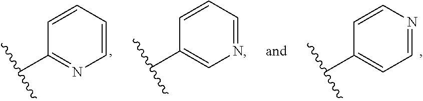

any of which is optionally substituted with one or more R¹⁵. In some embodiments, R⁶ is

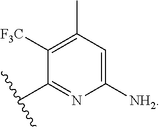 [SMILES: *c1nc(N)cc(C)c1C(F)(F)F]

In some embodiments, for a compound according to Formula VI, R⁶ is a bicyclic heteroaryl that is substituted with one or more R¹⁵. In some embodiments, R⁶ is selected from:

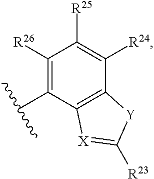

wherein X is selected from N and C—CN; Y is selected from O and S; R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and R²⁴, R²⁵, and R²⁶ are independently selected from H, deuterium, halogen, and C₁₋₆alkyl, wherein any C₁₋₆ alkyl is unsubstituted or substituted with one or more R¹³. In some embodiments, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R¹²)₂. In some embodiments, X is C—CN, Y is S, and R²³ is —NH₂. In some embodiments, R²⁴ is halogen (e.g., fluoro). In some embodiments, R²⁶ is deuterium.

In some embodiments, for a compound according to Formula VI, R⁶ is selected from:

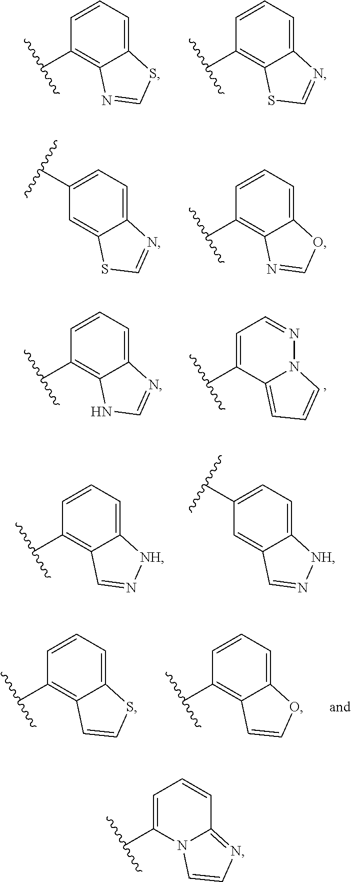

any of which is substituted with one or more R¹⁵.

In some embodiments, for a compound according to Formula VI, R⁶ is selected from:

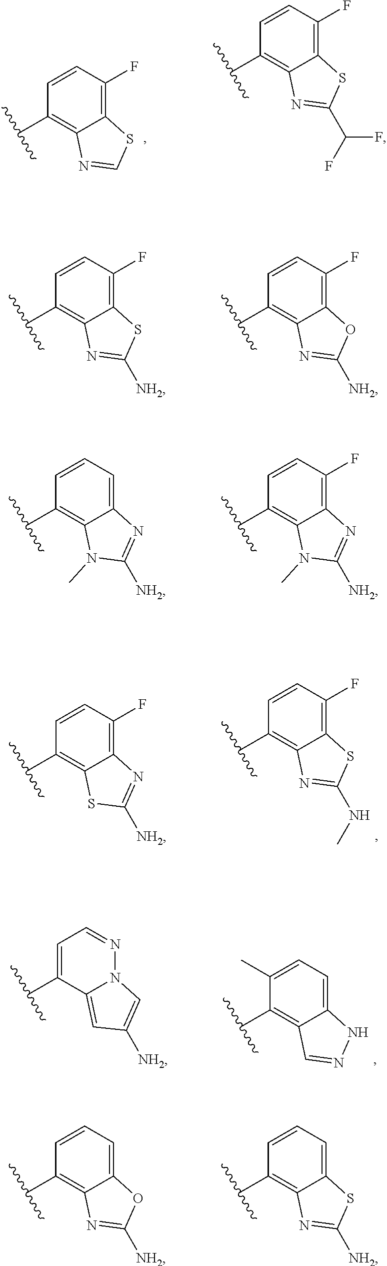

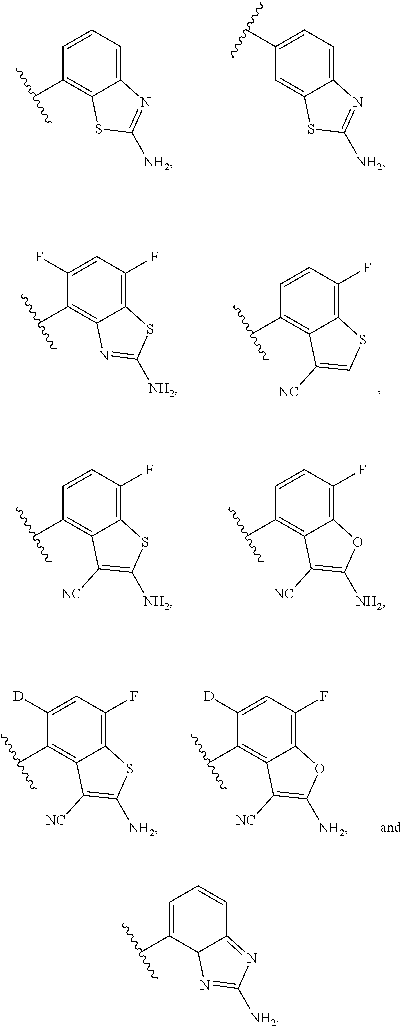

In some embodiments, for a compound according to Formula VI, R¹ is selected from:

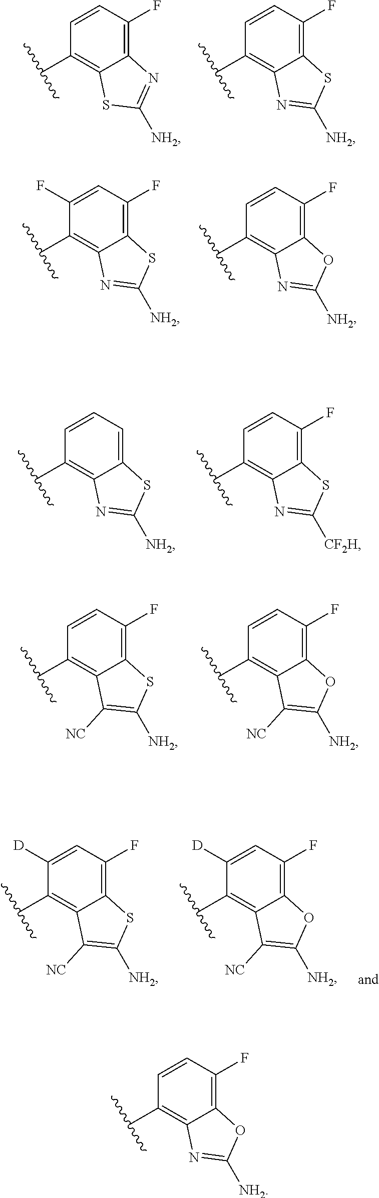

In some embodiments, for a compound according to Formula V1, R⁶ is

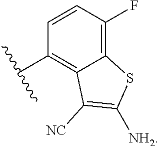 [SMILES: *c1ccc(F)c2sc(N)c(C#N)c12]

In some embodiments, for a compound of Formula VI or VI-a, R² is H. In some embodiments, R² is a C₁₋₆alkyl that is unsubstituted or is substituted with one or more R¹³. In some embodiments, R² is a C₁₋₆alkyl that is unsubstituted. In some embodiments, R² is a C₁₋₆alkyl that is substituted with one or more R¹³. In some embodiments, R² is a C₁₋₂alkyl that is unsubstituted or is substituted with one or more R¹³. In some embodiments, R² is a C₁₋₂alkyl that is unsubstituted. In some embodiments, R² is a C₁₋₂alkyl that is substituted with one or more R¹³. In some embodiments, R² is methyl. In some embodiments, R² is ethyl. In some embodiments, R² is a 3-6 membered carbocycle (e.g., cyclopropane).

In some embodiments, for a compound of Formula VI, R¹ is selected from —OR⁸, wherein R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl. In some embodiments, R⁸ is a heterocycle or an alkylheterocycle, wherein any heterocycle contains 4-8 members and is substituted with one or more R^{a} or R^{b}. In some embodiments, R⁸ is a heterocycle that is unsubstituted or substituted with one or more R^{a} or R^{b}. In some embodiments, R⁸ is an alkylheterocycle that is unsubstituted or substituted with one or more R^{a} or R^{b}. In some embodiments, R⁸ is —CH₂(heterocycle), where the heterocycle is unsubstituted or substituted with one or more R^{a} or R^{b}. In some embodiments, a heterocycle or a heterocycle of an alkylheterocycle is a 4-6 membered monocyclic heterocycle having 1-2 heteroatoms independently selected from N, O, and S. In some embodiments, a heterocycle or a heterocycle of an alkylheterocycle is an 8-membered bicyclic heterocycle having 1-2 heteroatoms independently selected from N, O, and S. In some embodiments, a heterocycle or a heterocycle of an alkylheterocycle is substituted with one or more R^{a} or R^{b}, wherein the one or more R^{a} or R^{b} is a halogen (e.g., F). In some embodiments, a heterocycle or a heterocycle of an alkylheterocycle is substituted with one or more R^{a} or R^{b}, wherein the one or more R^{a} or R^{b} is a C₁₋₆alkyl (e.g., methyl). In some embodiments, a heterocycle or a heterocycle of an alkylheterocycle is substituted with one or more R^{a} or R^{b}, wherein the one or more R^{a} or R^{b} is a —OR² (e.g., —OCH₃). In some embodiments, a heterocycle or a heterocycle of an alkylheterocycle is substituted with one or more R^{a} or R^{b}, wherein the one or more R^{a} or R^{b} is a 3-6 membered carbocycle (e.g., a cyclopropane). In some embodiments, a heterocycle or a heterocycle of an alkylheterocycle is substituted with one or more R^{a} or R^{b}, wherein the one or more R^{a} or R^{b} is deuterium.

In some embodiments, for a compound according to Formula VI, R¹ is selected from:

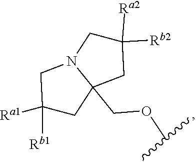

wherein R^{a1}, R^{a2}, R^{b1}, and R^{b2} are each independently selected from deuterium, halogen, C₁₋₆alkyl, —OR¹², and H, wherein R^{a1} and R^{b1} and/or R^{a2} and R^{b2} can optionally join together to form a 3-6 membered carbocycle, and wherein any C₁₋₆alkyl or 3-6 membered carbocycle is unsubstituted or is substituted with one or more R¹³. In some embodiments, R^{a1} and/or R^{a2} is a halogen. In some embodiments, R^{a1} and/or R^{a22} is F. In some embodiments, R^{a1} and/or R^{a2} is C₁₋₆alkyl that is unsubstituted or is substituted with one or more R¹³. In some embodiments, R^{a1} and/or R^{a2} is methyl. In some embodiments, R^{a1} and/or R^{a2} is —OC₁₋₆alkyl. In some embodiments, R^{a1} and/or R^{a2} is H. In some embodiments, R^{b1} and/or R^{b2} is H. In some embodiments, R^{b1} and/or R^{b2} is a halogen. In some embodiments, R^{b1} and/or R^{b2} is F. In some embodiments, R^{b1} and/or R^{b2} is C₁₋₆alkyl that is unsubstituted or is substituted with one or more R¹³. In some embodiments, R^{b1} and/or R^{b2} is methyl. In some embodiments, each of R^{a1} and R^{b1} is F. In some embodiments, R^{a2} and/or R^{b2} is D. In some embodiments, R^{a2} and R^{b2} join together to form a 3-6 membered carbocycle (e.g., cyclopropane), which carbocycle is optionally substituted with one or more R¹³. In some embodiments, R^{a1} and R^{b1} join together to form a 3-6 membered carbocycle (e.g., cyclopropane). In some embodiments, R¹ is selected from:

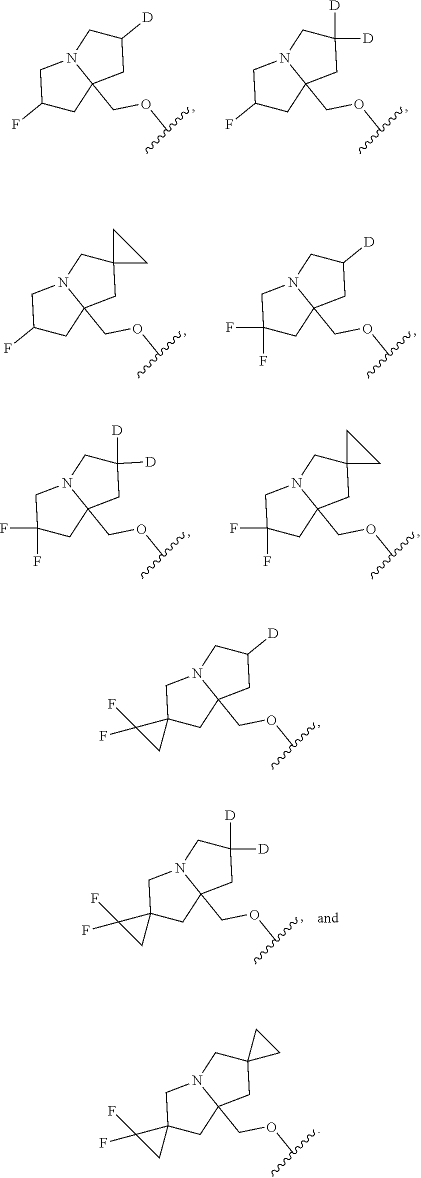

In some embodiments, for a compound of Formula VI, R¹ is selected from:

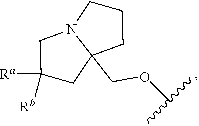

wherein R^{a} and R^{b} are each independently selected from halogen, C₁₋₆alkyl, —OR¹², and H, wherein any C₁₋₆ alkyl is unsubstituted or is substituted with one or more R¹³. In some embodiments, R⁵ is a halogen. In some embodiments, R^{a} is F. In some embodiments, R^{a} is C₁₋₆alkyl that is unsubstituted or is substituted with one or more R¹³. In some embodiments, R⁵ is methyl. In some embodiments, RU is —OC₁₋₆alkyl. In some embodiments, R^{a} is H. In some embodiments, R^{b} is H. In some embodiments, R^{b} is a halogen. In some embodiments, R^{b} is F. In some embodiments, R^{b} is C₁₋₆alkyl that is unsubstituted or is substituted with one or more R¹³. In some embodiments, R^{b} is methyl. In some embodiments, each of R^{a} and R^{b} is F. In some embodiments, each of R^{a} and R^{b} is methyl. In some embodiments, R¹ is selected from:

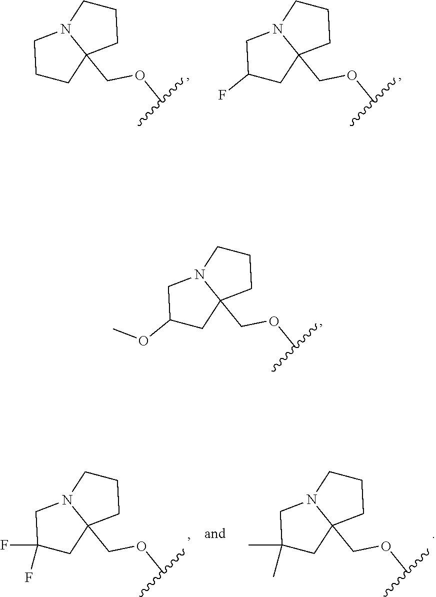 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

In some embodiments, for a compound of Formula VI, R¹ is selected from:

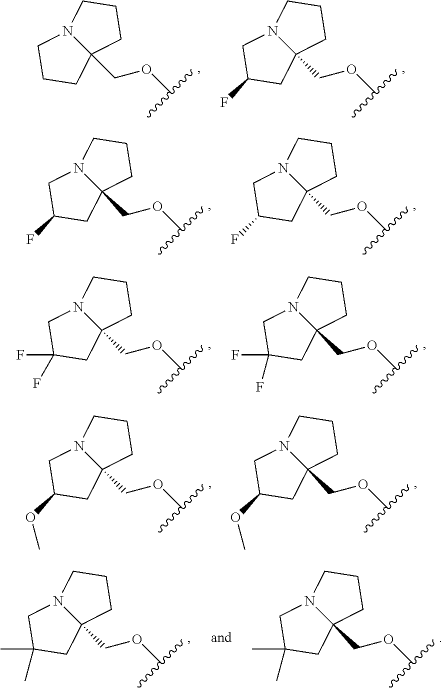

In some embodiments, for a compound of Formula VI, R¹ is selected from:

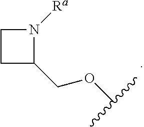

In some embodiments, R⁵ is C₁₋₆alkyl that is unsubstituted or substituted with one or more R¹³. In some embodiments, R^{a} is methyl. In some embodiments, R¹ is selected from:

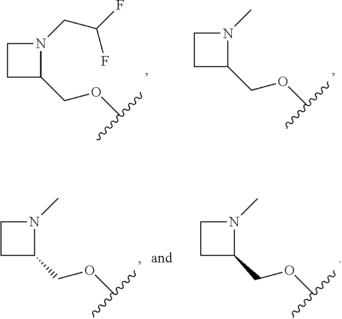

In some embodiments, for a compound of Formula VI, R¹ is selected from:

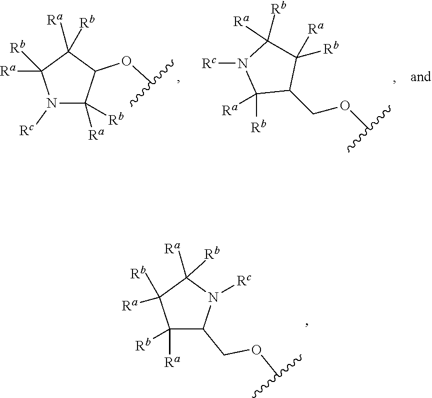

wherein each R^{a} and R^{b} is independently selected from halogen, C₁₋₆ alkyl, —OR¹⁴, and H; and R^{c} is selected from C₁₋₆alkyl, wherein an R^{a} and R^{b} or R^{c} optionally join together to form a 3-6 membered carbocycle or heterocycle, and wherein any C₁₋₆alkyl or 3-6 membered carbocycle or heterocycle is unsubstituted or is substituted with one or more R¹³. In some embodiments, each R^{a} and R^{b} is independently selected from halogen, C₁₋₆ alkyl, —OR¹⁴, and H; and R^{c} is selected from C₁₋₆ alkyl, wherein any C₁₋₆ alkyl is unsubstituted or is substituted with one or more R¹³, and wherein an R^{a} and R^{b} attached to the same carbon atom join together to form a 3-6 membered carbocycle. In some embodiments, each R^{a} and R^{b} is independently selected from halogen, C₁₋₆ alkyl, —OR¹⁴, and H; and wherein an R^{a} and R^{c} join together to form a 3-6 membered heterocycle. In some embodiments, each R^{a} and R^{b} is independently selected from halogen, C₁₋₆alkyl, —OR¹², and H; and R^{c} is selected from C₁₋₆ alkyl, wherein any C₁₋₆ alkyl is unsubstituted or is substituted with one or more R¹³. In some embodiments, one R^{a} or R^{b} is selected from halogen, C₁₋₆alkyl, and —OR¹², and the other R^{a} and R^{b} groups are H. In some embodiments, one R^{a} or R^{b} is halogen (e.g., F). In some embodiments, two R^{a} groups, two R^{b} groups, or an R^{a} and an R^{b} are halogen (e.g., F). In some embodiments, one R^{a} or R^{b} is —OR¹² (e.g., —OCH₃ or —CHF₂). In some embodiments, one R^{a} or R^{b} is C₁₋₆alkyl (e.g., methyl). In some embodiments, two R^{a} groups, two R^{b} groups, or an R^{a} and an R^{b} a C₁₋₆alkyl (e.g., methyl). In some embodiments, R^{c} is selected from —CH₃, —CH₂CH₂F, —CH₂CHF₂, and —CH₂CH₂CN. In some embodiments, an R^{a} and R^{b} join together to form a 3-6 membered carbocycle, such as a cyclopropane. In some embodiments, an R^{a} and R^{b} attached to the same carbon atom join together to form a 3-6 membered carbocycle, such as a cyclopropane. In some embodiments, an R^{a} and R^{c} join together to form a 3-6 membered heterocycle. In some embodiments, R¹ is selected from:

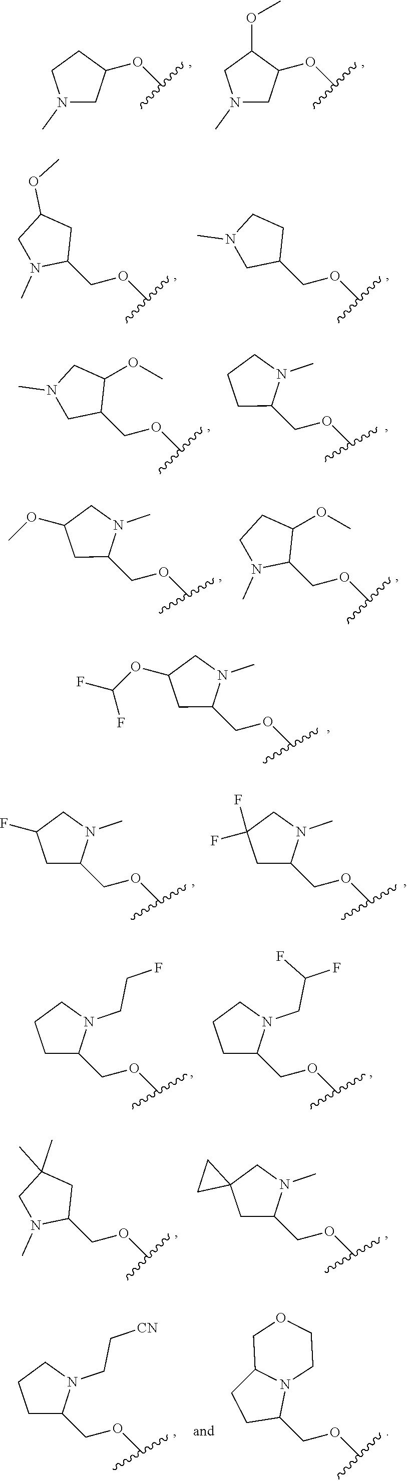

In some embodiments, for a compound of Formula VI, R¹ is selected from:

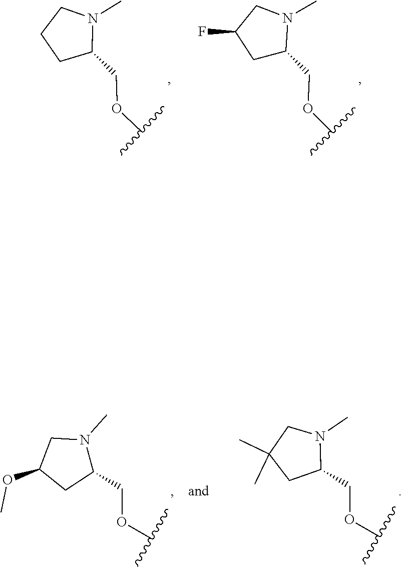

In some embodiments, for a compound of Formula VI, R¹ is selected from:

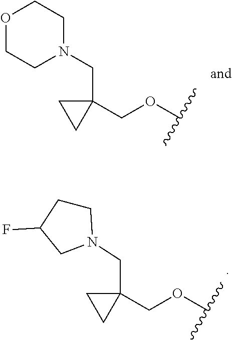 [SMILES: *OCC1(CN2CCOCC2)CC1 ; *OCC1(CN2CCC(F)C2)CC1]

In some embodiments, for a compound of Formula VI, R⁵ is H. In some embodiments, R⁵ is a halogen (e.g., F or Cl). In some embodiments, R⁵ is Cl. In some embodiments, R⁵ is F. In some embodiments, R¹ is —CN. In some embodiments, R³ is —OR¹², wherein R¹² is selected from C₁₋₆ alkyl and H. In some embodiments, R⁵ is —OCH₃. In some embodiments, R⁵ is —OCF₃. In some embodiments, R⁵ is selected from C₁₋₆alkyl that is unsubstituted or substituted with one or more R¹³. In some embodiments, R⁵ is selected from C₁₋₂alkyl that is unsubstituted or substituted with one or more R¹³. In some embodiments, R⁵ is selected from C₁₋₆alkyl that is unsubstituted, such as methyl or ethyl. In some embodiments, R³ is selected from C₁₋₆alkyl that is substituted with one or more halogens or —CN. In some embodiments, R¹ is C₁₋₆alkyl that is substituted with one or more halogens, such as one or more fluorines. In some embodiments, R⁵ is —CF₃. In some embodiments, R⁵ is —CHF₂. In some embodiments, R⁵ is selected from —CF₂H, —CF₃, —CH₂CN, and —CH₂CH₃. In some embodiments, R⁵ is selected from —CH₃, —CH₂CH₃, —CF₂H, —CF₃, —CF₂CH₃, and —CH₂CN. In some embodiments, R⁵ is C₁₋₆alkyl that is substituted with one or more R¹³, wherein each R¹³ is independently selected from —OR¹⁴, —CN, and —N(R¹⁴)₂. In some embodiments, R⁵ is —CH₂CN. In some embodiments, R⁵ is a 3-6 membered heterocycle. In some embodiments, R⁵ is a 5-6 membered heteroaryl, such as a furan.

In some embodiments, for a compound of Formula VI, R⁷ is Cl. In some embodiments, R⁷ is F. In some embodiments, R⁷ is a halogen. In some embodiments, R⁷ is H

In some embodiments, for a compound of Formula VI, the compound is a not a compound included in Table 4, or a salt, ester, tautomer, prodrug, zwitterionic form, or stereoisomer(s) thereof.

TABLE 4

| Compound | |
|---|---|
| Number | Structure |
| VI-308a | 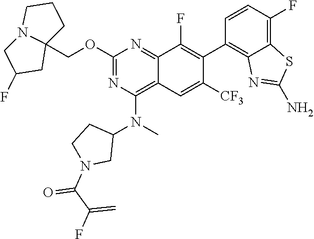 [SMILES: C=C(F)C(=O)N1CCC(N(C)c2nc(OCC34CCCN3CC(F)C4)nc3c(F)c(-c4ccc(F)c5sc(N)nc45)c(C(F)(F)F)cc23)C1] |

Also provided herein are embodiments wherein any embodiment described herein may be combined with any one or more of these embodiments, provided the combination is not mutually exclusive. As used herein, two embodiments are “mutually exclusive” when one is defined to be something which is different than the other. For example, an embodiment wherein two groups combine to form a ring is mutually exclusive with an embodiment in which one group is ethyl and the other group is hydrogen. Similarly, an embodiment wherein one group is CH₂ is mutually exclusive with an embodiment wherein the same group is NH.

In some embodiments of any of the preceding aspects, the compound is a compound included in Table 5, or a salt, ester, tautomer, prodrug, zwitterionic form, or stereoisomer(s) thereof. In some embodiments, the compound is a compound included in Table 5, or a salt (e.g., a pharmaceutically acceptable salt) thereof. In some embodiments of any of the preceding aspects, the compound is a compound included in Table 6, or a salt, ester, tautomer, prodrug, zwitterionic form, or stereoisomer(s) thereof. In some embodiments, the compound is a compound included in Table 6, or a salt (e.g., a pharmaceutically acceptable salt) thereof. In some embodiments of any of the preceding aspects, the compound is a compound included in Table 7, or a salt, ester, tautomer, prodrug, zwitterionic form, or stereoisomer(s) thereof. In some embodiments, the compound is a compound included in Table 7, or a salt (e.g., a pharmaceutically acceptable salt) thereof. In some embodiments of any of the preceding aspects, the compound is a compound included in Table 8, or a salt, ester, tautomer, prodrug, zwitterionic form, or stereoisomer(s) thereof. In some embodiments, the compound is a compound included in Table 8, or a salt (e.g., a pharmaceutically acceptable salt) thereof. In some embodiments of any of the preceding aspects, the compound is a compound included in Table 9, or a salt, ester, tautomer, prodrug, zwitterionic form, or stereoisomer(s) thereof. In some embodiments, the compound is a compound included in Table 9, or a salt (e.g., a pharmaceutically acceptable salt) thereof. In some embodiments of any of the preceding aspects, the compound is a compound included in Table 10, or a salt, ester, tautomer, prodrug, zwitterionic form, or stereoisomer(s) thereof. In some embodiments, the compound is a compound included in Table 10, or a salt (e.g., a pharmaceutically acceptable salt) thereof.

Also provided herein is a compound selected from Table 5, Table 6, Table 7, Table 8, Table 9, or Table 10 or any of the Examples provided herein, or a salt, ester, tautomer, prodrug, zwitterionic form, or stereoisomer(s) thereof. In some embodiments, the present disclosure provides a compound selected from Table 5, Table 6, Table 7, Table 8, Table 9, or Table 10 or any of the Examples provided herein, or a salt thereof.

In some embodiments of any of the preceding aspects, a compound provided herein, or a salt, ester, tautomer, zwitterionic form, or stereoisomer(s) thereof, is capable of interacting with a residue at the 12 and/or 13 and/or 61 position of the KRAS protein (e.g., a glutamine, histidine, cysteine, valine, aspartic acid, serine, alanine, arginine, or glycine residue). In some embodiments, a compound provided herein, or a salt, ester, tautomer, zwitterionic form, or stereoisomer(s) thereof, is capable of non-covalently interacting with a residue at the 12 and/or 13 and/or 61 position of the KRAS protein (e.g., a glutamine, histidine, cysteine, valine, aspartic acid, serine, alanine, arginine, or glycine residue), such as via one or more van der Waals, hydrogen bonding, ionic, or other interactions.

In some embodiments of any of the preceding aspects, a compound provided herein, or a salt, ester, tautomer, zwitterionic form, or stereoisomer(s) thereof, binds selectively to KRAS having a G12C mutation relative to KRAS having other residues at the 12 position of the P loop, such as arginine (R), glycine (G), valine (V), serine (S), alanine (A), and aspartic acid (D). For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater selectivity for KRAS having a G12C mutation relative to KRAS having other residues at the 12 position of the P loop, such as arginine (R), glycine (G), valine (V), serine (S), alanine (A), and aspartic acid (D). In some embodiments of any of the preceding aspects, a compound provided herein, or a salt, ester, tautomer, zwitterionic form, or stereoisomer(s) thereof, binds selectively to KRAS having a G12C mutation relative to wild-type KRAS. For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater binding selectivity for KRAS having a G12C mutation relative to wild-type KRAS. In some embodiments of any of the preceding aspects, a compound provided herein, or a salt, ester, tautomer, zwitterionic form, or stereoisomer(s) thereof, binds selectively to KRAS having a G12C mutation relative to other forms of RAS (e.g., HRAS and NRAS). For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater binding selectivity for KRAS having a G12C mutation relative to another form of RAS (e.g., HRAS or NRAS), such as an HRAS or NRAS protein having a G12C mutation. In some embodiments of any of the preceding aspects, a compound provided herein, or a salt, ester, tautomer, zwitterionic form, or stereoisomer(s) thereof, is capable of binding to a KRAS protein having a G12C mutation and one or more additional mutations, such as a mutation at codon 13 (to, e.g., D or C) or codon 61.

In some embodiments of any of the preceding aspects, a compound provided herein, or a salt, ester, tautomer, zwitterionic form, or stereoisomer(s) thereof, binds selectively to KRAS having a G12R mutation relative to KRAS having other residues at the 12 position of the P loop, such as cysteine (C), glycine (G), valine (V), serine (S), alanine (A), and aspartic acid (D). For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater selectivity for KRAS having a G12R mutation relative to KRAS having other residues at the 12 position of the P loop, such as cysteine (C), glycine (G), valine (V), serine (S), alanine (A), and aspartic acid (D). In some embodiments of any of the preceding aspects, a compound provided herein, or a salt, ester, tautomer, zwitterionic form, or stereoisomer(s) thereof, binds selectively to KRAS having a G12R mutation relative to wild-type KRAS. For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater binding selectivity for KRAS having a G12R mutation relative to wild-type KRAS. In some embodiments of any of the preceding aspects, a compound provided herein, or a salt, ester, tautomer, zwitterionic form, or stereoisomer(s) thereof, binds selectively to KRAS having a G12R mutation relative to other forms of RAS (e.g., HRAS and NRAS). For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater binding selectivity for KRAS having a G12R mutation relative to another form of RAS (e.g., HRAS or NRAS), such as an HRAS or NRAS protein having a G12R mutation. In some embodiments of any of the preceding aspects, a compound provided herein, or a salt, ester, tautomer, zwitterionic form, or stereoisomer(s) thereof, is capable of binding to a KRAS protein having a G12R mutation and one or more additional mutations, such as a mutation at codon 13 (to, e.g., D or C) or codon 61.

In some embodiments of any of the preceding aspects, a compound provided herein, or a salt, ester, tautomer, zwitterionic form, or stereoisomer(s) thereof, binds selectively to KRAS having a G12V mutation relative to KRAS having other residues at the 12 position of the P loop, such as cysteine (C), glycine (G), arginine (R), serine (S), alanine (A), and aspartic acid (D). For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater selectivity for KRAS having a G12V mutation relative to KRAS having other residues at the 12 position of the P loop, such as cysteine (C), glycine (G), arginine (R), serine (S), alanine (A), and aspartic acid (D). In some embodiments of any of the preceding aspects, a compound provided herein, or a salt, ester, tautomer, zwitterionic form, or stereoisomer(s) thereof, binds selectively to KRAS having a G12V mutation relative to wild-type KRAS. For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater binding selectivity for KRAS having a G12V mutation relative to wild-type KRAS. In some embodiments of any of the preceding aspects, a compound provided herein, or a salt, ester, tautomer, zwitterionic form, or stereoisomer(s) thereof, binds selectively to KRAS having a G12V mutation relative to other forms of RAS (e.g., HRAS and NRAS). For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater binding selectivity for KRAS having a G12V mutation relative to another form of RAS (e.g., HRAS or NRAS), such as an HRAS or NRAS protein having a G12V mutation. In some embodiments of any of the preceding aspects, a compound provided herein, or a salt, ester, tautomer, zwitterionic form, or stereoisomer(s) thereof, is capable of binding to a KRAS protein having a G12V mutation and one or more additional mutations, such as a mutation at codon 13 (to, e.g., D or C) or codon 61.

In some embodiments of any of the preceding aspects, a compound provided herein, or a salt, ester, tautomer, zwitterionic form, or stereoisomer(s) thereof, binds selectively to KRAS having a G12S mutation relative to KRAS having other residues at the 12 position of the P loop, such as cysteine (C), glycine (G), arginine (R), valine (V), alanine (A), and aspartic acid (D). For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater selectivity for KRAS having a G12S mutation relative to KRAS having other residues at the 12 position of the P loop, such as cysteine (C), glycine (G), arginine (R), valine (V), alanine (A), and aspartic acid (D). In some embodiments of any of the preceding aspects, a compound provided herein, or a salt, ester, tautomer, zwitterionic form, or stereoisomer(s) thereof, binds selectively to KRAS having a G12S mutation relative to wild-type KRAS. For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater binding selectivity for KRAS having a G12S mutation relative to wild-type KRAS. In some embodiments of any of the preceding aspects, a compound provided herein, or a salt, ester, tautomer, zwitterionic form, or stereoisomer(s) thereof, binds selectively to KRAS having a G12S mutation relative to other forms of RAS (e.g., HRAS and NRAS). For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater binding selectivity for KRAS having a G12S mutation relative to another form of RAS (e.g., HRAS or NRAS), such as an HRAS or NRAS protein having a G12S mutation. In some embodiments of any of the proceeding aspects, a compound provided herein, or a salt, ester, tautomer, zwitterionic form, or stereoisomer(s) thereof, is capable of binding to a KRAS protein having a G12S mutation and one or more additional mutations, such as a mutation at codon 13 (to, e.g., D or C) or codon 61.

In some embodiments of any of the preceding aspects, a compound provided herein, or a salt, ester, tautomer, zwitterionic form, or stereoisomer(s) thereof, binds selectively to KRAS having a G12A mutation relative to KRAS having other residues at the 12 position of the P loop, such as cysteine (C), glycine (G), arginine (R), valine (V), serine (S), and aspartic acid (D). For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater selectivity for KRAS having a G12A mutation relative to KRAS having other residues at the 12 position of the P loop, such as cysteine (C), glycine (G), arginine (R), valine (V), serine (S), and aspartic acid (D). In some embodiments of any of the preceding aspects, a compound provided herein, or a salt, ester, tautomer, zwitterionic form, or stereoisomer(s) thereof, binds selectively to KRAS having a G12A mutation relative to wild-type KRAS. For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater binding selectivity for KRAS having a G12A mutation relative to wild-type KRAS. In some embodiments of any of the preceding aspects, a compound provided herein, or a salt, ester, tautomer, zwitterionic form, or stereoisomer(s) thereof, binds selectively to KRAS having a G12A mutation relative to other forms of RAS (e.g., HRAS and NRAS). For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater binding selectivity for KRAS having a G12A mutation relative to another form of RAS (e.g., HRAS or NRAS), such as an HRAS or NRAS protein having a G12A mutation. In some embodiments of any of the preceding aspects, a compound provided herein, or a salt, ester, tautomer, zwitterionic form, or stereoisomer(s) thereof, is capable of binding to a KRAS protein having a G12A mutation and one or more additional mutations, such as a mutation at codon 13 (to, e.g., D or C) or codon 61.

In some embodiments of any of the preceding aspects, a compound provided herein, or a salt, ester, tautomer, zwitterionic form, or stereoisomer(s) thereof, binds selectively to KRAS having a G12D mutation relative to KRAS having other residues at the 12 position of the P loop, such as cysteine (C), glycine (G), arginine (R), valine (V), serine (S), and alanine (A). For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater selectivity for KRAS having a G12D mutation relative to KRAS having other residues at the 12 position of the P loop, such as cysteine (C), glycine (G), arginine (R), valine (V), serine (S), and alanine (A). In some embodiments of any of the preceding aspects, a compound provided herein, or a salt, ester, tautomer, zwitterionic form, or stereoisomer(s) thereof, binds selectively to KRAS having a G12D mutation relative to wild-type KRAS. For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater binding selectivity for KRAS having a G12D mutation relative to wild-type KRAS. In some embodiments of any of the preceding aspects, a compound provided herein, or a salt, ester, tautomer, zwitterionic form, or stereoisomer(s) thereof, binds selectively to KRAS having a G12D mutation relative to other forms of RAS (e.g., HRAS and NRAS). For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater binding selectivity for KRAS having a G12D mutation relative to another form of RAS (e.g., HRAS or NRAS), such as an HRAS or NRAS protein having a G12D mutation. In some embodiments of any of the preceding aspects, a compound provided herein, or a salt, ester, tautomer, zwitterionic form, or stereoisomer(s) thereof, is capable of binding to a KRAS protein having a G12D mutation and one or more additional mutations, such as a mutation at codon 13 (to, e.g., D or C) or codon 61.

In some embodiments of any of the preceding aspects, a compound provided herein, or a salt, ester, tautomer, zwitterionic form, or stereoisomer(s) thereof, binds selectively to KRAS having a G13D mutation relative to KRAS having other residues at the 13 position of the P loop, such as cysteine (C), glycine (G), arginine (R), valine (V), serine (S), and alanine (A). For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater selectivity for KRAS having a G13D mutation relative to KRAS having other residues at the 13 position of the P loop, such as cysteine (C), glycine (G), arginine (R), valine (V), serine (S), and alanine (A). In some embodiments of any of the preceding aspects, a compound provided herein, or a salt, ester, tautomer, zwitterionic form, or stereoisomer(s) thereof, binds selectively to KRAS having a G13D mutation relative to wild-type KRAS.

For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater binding selectivity for KRAS having a G13D mutation relative to wild-type KRAS. In some embodiments of any of the preceding aspects, a compound provided herein, or a salt, ester, tautomer, zwitterionic form, or stereoisomer(s) thereof, binds selectively to KRAS having a G13D mutation relative to other forms of RAS (e.g., HRAS and NRAS). For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater binding selectivity for KRAS having a G13D mutation relative to another form of RAS (e.g., HRAS or NRAS), such as an HRAS or NRAS protein having a G13D mutation. In some embodiments of any of the preceding aspects, a compound provided herein, or a salt, ester, tautomer, zwitterionic form, or stereoisomer(s) thereof, is capable of binding to a KRAS protein having a G13D mutation and one or more additional mutations, such as a mutation at codon 12 (to, e.g., D, C, A, S, V, or R) or codon 61.

In some embodiments of any of the preceding aspects, a compound provided herein, or a salt, ester, tautomer, zwitterionic form, or stereoisomer(s) thereof, binds selectively to KRAS having a Q61H mutation relative to KRAS having other residues at the 61 position of the P loop, such as glutamine (Q). For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater selectivity for KRAS having a Q61H mutation relative to KRAS having other residues at the 61 position of the P loop, such as glutamine (Q). In some embodiments of any of the preceding aspects, a compound provided herein, or a salt, ester, tautomer, zwitterionic form, or stereoisomer(s) thereof, binds selectively to KRAS having a Q61H mutation relative to wild-type KRAS. For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater binding selectivity for KRAS having a Q61H mutation relative to wild-type KRAS. In some embodiments of any of the preceding aspects, a compound provided herein, or a salt, ester, tautomer, zwitterionic form, or stereoisomer(s) thereof, binds selectively to KRAS having a Q61H mutation relative to other forms of RAS (e.g., HRAS and NRAS). For example, in some embodiments, a compound provided herein, or a salt, ester, tautomer, zwitterionic form, or stereoisomer(s) thereof, demonstrates at least 1.5, 2, 3, 4, 5, 10, 20, 30, 40, 50, 60, 70, 80, 90, 100-fold, or greater binding selectivity for KRAS having a Q61H mutation relative to another form of RAS (e.g., HRAS or NRAS), such as an HRAS or NRAS protein having a Q61H mutation. In some embodiments of any of the preceding aspects, a compound provided herein, or a salt, ester, tautomer, zwitterionic form, or stereoisomer(s) thereof, is capable of binding to a KRAS protein having a Q61H mutation and one or more additional mutations, such as a mutation at codon 12 (to, e.g., D, C, A, S, V, or R) or codon 13 (to, e.g., D).

In some embodiments of any of the preceding aspects, a compound provided herein, or a salt, ester, tautomer, zwitterionic form, or stereoisomer(s) thereof, is capable of selectively binding a KRAS protein in an active (GTP-bound) conformation. In some embodiments of any of the preceding aspects, a compound provided herein, or a salt, ester, tautomer, zwitterionic form, or stereoisomer(s) thereof, is capable of selectively binding a KRAS protein in an inactive (GDP-bound) conformation. In some embodiments of any of the preceding aspects, a compound provided herein, or a salt, ester, tautomer, zwitterionic form, or stereoisomer(s) thereof, is capable of selectively binding a KRAS protein in both active (GTP-bound) and inactive (GDP-bound) conformations. In some embodiments of any of the preceding aspects, a compound provided herein, or a salt, ester, tautomer, zwitterionic form, or stereoisomer(s) thereof, has higher selectivity for a KRAS protein m its active (GTP-bound) conformation than in its inactive (GDP-bound) conformation. In some embodiments of any of the preceding aspects, a compound provided herein, or a salt, ester, tautomer, zwitterionic form, or stereoisomer(s) thereof, has higher selectivity for a KRAS protein in its inactive (GDP-bound) conformation than in its active (GTP-bound) conformation.

### Compositions

The present disclosure also provides a composition (e.g., a pharmaceutical composition) comprising a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, ID, IID1, IIIE, IIE1, IIF, II, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IV, IIV1, IIW, IIW1, IIX, IIX1, IIY, IIY1, IIZ, IIZ1, IIAA, IIAA1, III, III′, III-a, IIIA, IIIA1, IIIB, IIIC, IIIC, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof. In some embodiments, a provided composition comprises a compound provided herein, or a pharmaceutically acceptable salt thereof. For example, the present disclosure provides a pharmaceutical composition comprising a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IX, IIX1, IIY, IIY1, IIZ, IIZ1, IIAA, IIAA1, IIIQ, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof, together with a pharmaceutically acceptable carrier. In some embodiments, a provided pharmaceutical composition comprises a compound provided herein or a pharmaceutically acceptable salt thereof, together with a pharmaceutically acceptable carrier.

In some embodiments, the pharmaceutical composition is formulated for oral administration. In some embodiments, the oral pharmaceutical formulation is selected from a tablet and a capsule.

In some embodiments, the pharmaceutical composition is formulated for parenteral administration. In some embodiments, the pharmaceutical composition is formulated for intravenous administration. In some embodiments, the pharmaceutical composition is formulated for subcutaneous administration.

While it may be possible for certain compounds provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, ID1, IIE, IIE1, IIF, IIF1, IG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, III, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof, to be administered as the raw chemical, compounds may additionally or alternatively be provided in a pharmaceutical formulation. Accordingly, provided herein are pharmaceutical formulations which comprise one or more compounds disclosed herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ1, IIQ1, IIR1, II1S, IIS1, IIT, IIT1, IIU, IIU1, IIV, IIV1, IIW, IIW1, IIX, IIX1, IIY, IIY1, IIZ, IIZ1, IIAA, IIAA1, II, II′, III-a, IIA, IIIA1, IIIB, IIIC, IIIC1, IIID, IIIE, IIIF, IIIG, IIIH, IIIJ, IIK, IIIL, IIIM, IIIN, IIIP, IIIQ, IIIR, IIIS, IIIT, IV, IV-a, IVA, IVB, IVC, V, V-a, VA, VB, VC, VI, and VI-a), or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration selected. Any of the well-known techniques, carriers, and excipients may be used as suitable and as understood in the art. The pharmaceutical compositions disclosed herein may be manufactured in any suitable manner known, e.g., by means of conventional mixing, dissolving, granulating, dragee-making, levigating, emulsifying, encapsulating, entrapping or compression processes.

A pharmaceutical formulation provided herein can be suitable for oral, parenteral (including subcutaneous, intradermal, intramuscular, intravenous, intraarticular, and intramedullary), intraperitoneal, transmucosal, transdermal, rectal, and topical (including dermal, buccal, sublingual, and intraocular) administration. The most suitable route may depend on, for example, the condition and disorder of the subject to which the pharmaceutical formulation will be administered. A pharmaceutical formulation can be provided in a unit dosage form. A pharmaceutical formulation can be prepared by any suitable method. A method of preparing a pharmaceutical formulation may comprise bringing a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IB1, IIC, IIC1, IID, IID1, IIE, IIE1, IIF, IIF1, IIG, IIG1, IIH, IIH1, IIJ, IIJ1, IIK, IIK1, IIL, IIL1, IM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, IIIB, IIIC, III-a, IIIA, IIIA1, IIIB, IIIC, IIIC, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a pharmaceutically acceptable salt, ester, amide, prodrug or solvate thereof (“active ingredient”) in contact with one or more pharmaceutically acceptable carriers (e.g., accessory ingredients). In general, the formulations are prepared by uniformly and intimately bringing into association the active ingredient with liquid carriers or finely divided solid carriers or both and then, if necessary, shaping the product into the desired formulation.

Pharmaceutical formulations of compounds provided herein (e.g., compounds of any one of Formulas I, I-a, IA, IA1, IA2, IB, B1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IF1, IIG, IIG1, IIH, IIH1, IIL, IIL1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, III, III′, III-a, IIIA1, IIIB, IIIC, IIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a in any available form (e.g., salt, ester, tautomer, prodrug, zwitterionic form, stereoisomer(s) etc.)) may be provided as discrete units. For example, a formulation suitable for oral administration may be provided as capsules, cachets, and/or tablets containing a predetermined amount of the compound in any suitable form (e.g., the active ingredient); as a solution or suspension in a solvent (e.g., aqueous or non-aqueous solvent); as an emulsion (e.g., an oil-in-water liquid emulsion or water-in-oil liquid emulsion); or as a powder or granules. The active ingredient may additionally or alternatively be provided as a bolus, electuary, or paste.

Pharmaceutical preparations suitable for oral administration include tablets, push-fit capsules made of gelatin, as well as soft, scaled capsules made of gelatin and a plasticizer, such as glycerol or sorbitol. Tablets may be made by, for example, compression or molding, optionally with one or more accessory ingredients, such as one or more pharmaceutically acceptable excipients. Compressed tablets may be prepared by, for example, compressing in a suitable machine the active ingredient in a free-flowing form such as a powder or granules, optionally mixed with binders, inert diluents, or lubricating, surface active or dispersing agents. Molded tablets may be made by, for example, molding in a suitable machine a mixture of the powdered compound moistened with an inert liquid diluent. The tablets may optionally be coated or scored and may be formulated to provide slow or controlled release of the active ingredient therein. All formulations for oral administration should be in dosages suitable for such administration. The push-fit capsules can contain the active ingredients in admixture with, for example, one or more fillers such as lactose, one or more binders such as one or more starches, and/or one or more lubricants such as talc or magnesium stearate and, optionally, one or more stabilizers. In soft capsules, the active compounds may be dissolved or suspended in suitable liquids, such as fatty oils, liquid paraffin, or liquid polyethylene glycols. Stabilizers and other elements may also be added. Dragee cores are provided with suitable coatings. For this purpose, concentrated sugar solutions may be used, which may optionally contain a gum, gelling agent, polymer, solvent, or combination thereof. Dyestuffs or pigments may be added to the tablets or dragee coatings for identification or to characterize different combinations of active compound doses.

A pharmaceutical composition comprising a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II, II-a, IIA, IIA1, IIB, IIB1, IIC, IIC1, IID, IID1, IIE, IIE1, IIF, IIF1, IIG, IIG1, IIH, IIH1, IIU, IIJ, IIK, IIK1, IIL, IIL1, IIM, IIM1, IIN, IIN1, IIP, IIP1, IIQ, IIQ1, IR, IIR1, IS, IIS1, IIT, IIT1, IIU, IIU1, IIV, IIV1, IIW, IIW1, IIX, IIX1, IIY, IIY1, IIZ, IIZ1, IIAA, IIAA1, III, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a form thereof (e.g., salt, ester, tautomer, prodrug, zwitterionic form, stereoisomer(s), etc.), may be formulated for parenteral administration by injection, e.g., by bolus injection or continuous infusion. Formulations for injection may be presented in unit dosage form, e.g., in ampoules, vials, or in multi-dose containers, with an added preservative. The compositions may take such forms as suspensions, solutions, or emulsions in oily or aqueous vehicles, and may contain formulating agents such as suspending, stabilizing, and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, prior (e.g., immediately prior) to use. Extemporaneous injection solutions and suspensions may be prepared from sterile powders, granules and tablets of the kind previously described.

A pharmaceutical composition comprising a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IE, IIE1, IIF, IIF1, IIG, IIG1, IIH, IIH1, IIU, IIU1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, III, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IIIV, IV-a, IVA, IVB, IVC, V, V-a, VA, VB, VC, VI, and VI-a), or a form thereof (e.g., salt, ester, tautomer, prodrug, zwitterionic form, stereoisomer(s), etc.), may be formulated as a solution for injection, which solution may be an aqueous or non-aqueous (oily) sterile solution and may comprise one or more antioxidants, thickening agents, suspending agents, buffers, solutes, and/or bacteriostats. The addition of one or more such additives may render the formulation isotonic with the blood of the intended recipient (e.g., subject or patient). Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described elsewhere herein, the compounds provided herein (e.g., compounds of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, II, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIIA, IIIA1, III, III′, III-a, IIIA, IIIA1, IIIB, IIIC, IIIC1, IIID, IIIE, IIIf, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a in any suitable form (e.g., salt, ester, tautomer, prodrug, zwitterionic form, stereoisomer(s), etc.)), may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical composition comprising a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC, IID, IID1, IIE, IIE1, IIF, IIF1, IIG, IIG1, II, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, III, III′, III-a, IIIA, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a) or a form thereof (e.g., salt, ester, tautomer, prodrug, zwitterionic form, stereoisomer(s), etc.) that is suitable for buccal or sublingual administration may take the form of tablets, lozenges, pastilles, or gels. Such compositions may comprise the active ingredient in a flavored basis such as sucrose and acacia or tragacanth. A pharmaceutical composition comprising a compound provided herein or a form thereof (e.g., salt, ester, tautomer, prodrug, zwitterionic form, stereoisomer(s), etc.) that is suitable for rectal administration may be formulated as a suppository or retention enema and may comprise a medium such as, for example, cocoa butter, polyethylene glycol, or other glycerides.

Certain compounds provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, III, III′, III-a, IIIA, IIIA1, IIIB, IIIC, IIIC1, IIID, IIIE, IIIF, IIIG, IIIH, IIIU, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a) or a form thereof (e.g., salt, ester, tautomer, prodrug, zwitterionic form, stereoisomer(s), etc.) may be formulated for non-systemic administration, such as topical administration. This includes the application of a compound disclosed herein, or a form thereof, externally to the epidermis or the buccal cavity and the instillation of such a compound, or a form thereof, into the car, eye, and nose, such that the compound, or a form thereof, does not significantly enter the blood stream. In contrast, systemic administration refers to oral, intravenous, intraperitoneal, and intramuscular administration.

Formulations suitable for topical administration include liquid or semi-liquid preparations suitable for penetration through the skin to the site of inflammation such as gels, liniments, lotions, creams, ointments, or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds (e.g., compounds of any one of Formulas I, I-a, IA, IA1, IA2, IB, IB1, IB2, IC, IC1, IC2, ID, ID1, ID2, IE, IE1, IE2, IF, IF1, IF2, I, II′, II-a, IIA, IIA1, IIB, IIB1, IIC, IIC1, IID, IID1, IIE, IE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIHW, IIW1, IIX, IIX1, IIY, IIY1, IIZ, IIZ1, IIAA, IIAA1, III, III′, III-a, IIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a) or forms thereof (e.g., salt, ester, tautomer, prodrug, zwitterionic form, stereoisomer(s), etc.) may be conveniently delivered from an insufflator, nebulizer pressurized packs, or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Alternatively, for administration by inhalation or insufflation, the compounds provided herein may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as described herein, or an appropriate fraction thereof, of the active ingredient (e.g., a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, ID1, IIE, IIE1, IIF, IIF1, IIG, IIG1, IIHH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, III, III′, III-a, IIIA, IIIA1, IIIB, IIIC, IIIC1, IIID, IIIE, IIIF, IIIG, IIIH, IIIU, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof).

It should be understood that in addition to the ingredients particularly described elsewhere herein, the formulations described herein may include other useful agents having regard to the type of formulation in question, for example those suitable for oral administration may include flavoring agents. Compounds (e.g., compounds of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIT, IIV, IIV1, IIW, IIW1, IIX, IIX1, IIY, IIY1, IIZ, IIZ1, IIAA, IIAA1, III, III′, III-a, IIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a) or forms thereof (e.g., salt, ester, tautomer, prodrug, zwitterionic form, stereoisomer(s), etc.) may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

### Methods

The present disclosure also provides a method of modulating KRAS (e.g., KRAS having a Q61H, G12C, G12D, G12V, G12S, G12A, G12R, or G13D mutation, or wild-type KRAS) comprising contacting KRAS with a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, III, III′, III-a, IIIA, IIIA1, IIIB, IIIC, IIIC1, IIID, IIIE, IIHF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof. For example, the present disclosure may provide a method of altering a cell phenotype, cell proliferation, KRAS activity, biochemical output produced by active or inactive KRAS, expression of KRAS, and/or binding of KRAS with a natural binding partner. Any such feature may be monitored and may be altered upon contacting KRAS with a compound provided herein, or a form thereof. A method of modulating KRAS (e.g., KRAS having a Q61H, G12C, G12D, G12V, G12S, G12A, G12R, or G13D mutation, or wild-type KRAS) may be a mode of treatment of a disease, disorder, or condition (e.g., a cancer), a biological assay, a cellular assay, a biochemical assay, etc. In some embodiments, a method of modulating KRAS (e.g., KRAS having a Q61H, G12C. G12D, G12V, G12S, G12A. G12R, or G13D mutation, or wild-type KRAS) comprises contacting a KRAS protein with a compound provided herein, or a salt, ester, tautomer, zwitterionic form, or stereoisomer(s) thereof, where the KRAS protein is in the active (GTP-bound) conformation. In some embodiments, a method of modulating KRAS (e.g., KRAS having a Q61H, G12C, G12D, G12V, G12S, G12A, G12R, or G13D mutation, or wild-type KRAS) comprises contacting a KRAS protein with a compound provided herein, or a salt, ester, tautomer, zwitterionic form, or stereoisomer(s) thereof, where the KRAS protein is in the inactive (GDP-bound) conformation. In some embodiments, contacting a KRAS protein with a compound provided herein, or a salt, ester, tautomer, zwitterionic form, or stereoisomer(s) thereof, comprises incubating the KRAS protein with the compound or form thereof. In some embodiments, contacting a KRAS protein with a compound provided herein, or a salt, ester, tautomer, zwitterionic form, or stereoisomer(s) thereof, comprises contacting a cell containing the KRAS protein with the compound or form thereof. In some embodiments, the cell is in a subject. In some embodiments, the subject is a human. In some embodiments, the subject is a human having a disease, disorder, or condition such as a cancer, such as a cancer characterized by a KRAS protein having a Q61H, G12C, G12D, G12V, G12S, G12A, G12R, or G13D mutation, or wild-type KRAS.

The present disclosure also provides methods of treating a disease, disorder, or condition in a subject in need thereof using a compound provided herein, (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, III, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof. For example, the present disclosure provides a method comprising providing (e.g., administering) to a subject (e.g., patient) in need thereof an effective amount of a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IC1, IID, IID1, IIE, IIIE1, IIF, IIF1, IG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIHY, IIY1, IIZ, IIZ1, IIAA, IIAA1, III, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof. The present disclosure also provides methods of treating a disease, disorder, or condition in a subject in need thereof using a pharmaceutical composition comprising a compound provided herein, (e.g., a compound of any one of Formulas I, I-a, IA, IA1, A2, IB, IB1, IB2, IC, IC1, IC2, ID, ID1, ID2, IE, IE1, IE2, IF, IF1, IF2, II, II′, II-a, IIA, IA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, III, III′, III-a, IIIA, IIIA1, IIIB, IIIC, IIIC, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof. For example, the present disclosure provides a method comprising providing (e.g., administering) to a subject (e.g., patient) in need thereof a pharmaceutical composition comprising an effective amount of a compound provided herein (e.g., a compound of any one of Formulas I, I-a, IA, IA1, IA2, IB, IB1, IB2, IC, IC1, IC2, ID, ID1, ID2, IE, IE1, IE2, IF. IF1, IF2, II, I′, II-a, IIA, IIA1, IIB, IIB1, IIC, IIC1, ID, IID1, IIE, IIE1, IIF, IF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IV, IIV1, IIW, IIW1, IIX, IIX1, IIY, IIY1, IIZ, IIZ1, IIAA, IIAA1, III, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, V, IV-a, IVA, IVB, IVC, V, V-a, VA, VB, VC, VI, and VI-a), or a salt, ester, tautomer, prodrug, zwitterionic form, or stereoisomer(s) thereof. In some embodiments, the subject is known to have (e.g., has previously been diagnosed with) a disease, disorder, or condition such as a cancer. The disease, disorder, or condition may be a KRAS-mediated disease, such as a cancer characterized by a KRAS protein having a Q61H, G12C, G12D, G12V, G12S, G12A, G12R, or G13D mutation, or wild-type KRAS. In some embodiments, the compound administered to the subject in need thereof according to the methods described herein is a compound described in an embodiment, example, figure, or table herein, or a stereoisomer(s) or pharmaceutically acceptable salt thereof.

The present disclosure also provides a compound as provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IE, IIE1, IIF, IIF1, IIG, IIG1, IIH, IIH1, IIU, IIJ, IIK, IIK1, IIL, IIL1, IIM, IIM1, IIN, IIN1, IIP, IP1, IIQ, IIQ1, IIR, IIR1, IIS, IIS1, IIT, IIT1, IIU, IIU1, IV, IIV1, IIW, IIW1, IIX, IIX1, IIY, IIY1, IIZ, IIZ1, IIAA, IIAA1, III, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof, or a pharmaceutical composition comprising any of the foregoing compounds and a pharmaceutically acceptable excipient, for use as a medicament, such as a medicament for the treatment of a disease, disorder, or condition (e.g., a cancer). The present disclosure also provides a compound as provided herein (e.g., a compound of any one of Formulas I, I-a, IA, IA1, IA2, IB, IB1, IB2, IC, IC, IC2, ID, ID1, ID2, IE, IE1, IE2, IF, IF1, IF2, II, II′, II-a, IIA, IIA, IIB, IB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU, IIV, IIV1, IIW, IIW1, IIX, IX1, IIY, IIY1, IIZ, IIZ1, IIAA, IIAA1, III, III′, III-a, IIIA, IIIA1, IIIB, IIIC, IIIC, IIID, IIIE, IIIF, IIIG, IIIH, IIIU, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof, or a pharmaceutical composition comprising any of the foregoing compounds and a pharmaceutically acceptable excipient, for use in the manufacture of a medicament for the treatment of a disease, disorder, or condition (e.g., a cancer) in a subject in need thereof.

The present disclosure also provides the use of a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT, IIU, IIU1, IIV, IIV1, IIW, IIW1, IIX, IIX1, IIY, IIY1, IIZ, IIZ1, IIAA, IIAA1, III, III′, III-a, IIIA, IIIA1, IIIB, IIIC, IIIC1, IIID, IIIE, IIIF, IIIG, IIIH, IIIK, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof, or a pharmaceutical composition comprising any of the foregoing compounds and a pharmaceutically acceptable excipient, for the treatment of a disease, disorder, or condition (e.g., a cancer, as described herein, such as a cancer characterized by a KRAS protein having a Q61H, G12C, G12D, G12V, G12S, G12A, G12R, or G13D mutation, or wild-type KRAS) in a subject in need thereof.

The present disclosure also provides the use of a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIIE, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, III, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof, or a pharmaceutical composition comprising any of the foregoing compounds and a pharmaceutically acceptable excipient, in the manufacture of a medicament for treating a disease, disorder, or condition (e.g., a cancer, as described herein, such as a cancer characterized by a KRAS protein having a Q61H, G12C, G12D, G12V, G12S, G12A, G12R, or G13D mutation, or wild-type KRAS) in a subject in need thereof.

The present disclosure also provides a method of inhibiting KRAS (e.g., KRAS protein having a Q61H, G12C, G12D, G12V, G12S, G12A, G12R, or G13D mutation, or wild-type KRAS) (e.g., in a subject in need thereof) comprising contacting KRAS with a compound as provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIIE, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, III, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof, or a pharmaceutical composition comprising any of the foregoing compounds and a pharmaceutically acceptably excipient. In some embodiments, a method of inhibiting KRAS (e.g., KRAS protein having a Q61H, G12C, G12D, G12V. G12S, G12A, G12R, or G13D mutation, or wild-type KRAS) comprises contacting a KRAS protein with a compound provided herein, or a salt, ester, tautomer, zwitterionic form, or stereoisomer(s) thereof, where the KRAS protein is in the active (GTP-bound) conformation. In some embodiments, a method of inhibiting KRAS (e.g., KRAS protein having a Q61H, G12C, G12D, G12V, G12S, G12A, G12R, or G13D mutation, or wild-type KRAS) comprises contacting a KRAS protein with a compound provided herein, or a salt, ester, tautomer, zwitterionic form, or stereoisomer(s) thereof, where the KRAS protein is in the inactive (GDP-bound) conformation. In some embodiments, contacting a KRAS protein with a compound provided herein, or a salt, ester, tautomer, zwitterionic form, or stereoisomer(s) thereof, comprises incubating the KRAS protein with the compound or form thereof. In some embodiments, contacting a KRAS protein with a compound provided herein, or a salt, ester, tautomer, zwitterionic form, or stereoisomer(s) thereof, comprises contacting a cell containing the KRAS protein with the compound or form thereof. In some embodiments, the cell is in a subject. In some embodiments, the subject is a human. In some embodiments, the subject is a human having a disease, disorder, or condition such as a cancer, such as a cancer characterized by a KRAS protein having a Q61H, G12C, G12D, G12V, G12S, G12A, G12R, or G13D mutation, or wild-type KRAS.

The present disclosure also provides a compound as provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IV, IIV1, IIW, IIW1, IIX, IIX1, IIY, IIY1, IIZ, IIZ1, IIAA, IIAA1, III, III′, III-a, IIIA, IIIA1, IIIB, IIIC, IIIC1, IIID, IIIE, IIIF, IIIG, IIIH, IIIU, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof, or a pharmaceutical composition comprising any of the foregoing compounds and a pharmaceutically acceptable excipient, for use in inhibiting KRAS (e.g., KRAS protein having a Q61H, G12C, G12D, G12V, G12S, G12A, G12R, or G13D mutation, or wild-type KRAS) (e.g., in a subject in need thereof). The present disclosure also provides a compound as provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, II, II′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof, or a pharmaceutical composition comprising any of the foregoing compounds and a pharmaceutically acceptable excipient, for use in the manufacture of a medicament for inhibiting KRAS (e.g., KRAS protein having a Q61H, G12C, G12D, G12V, G12S, G12A, G12R, or G13D mutation, or wild-type KRAS) in a subject in need thereof.

The present disclosure also provides the use of a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, II, II′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof, or a pharmaceutical composition comprising any of the foregoing compounds and a pharmaceutically acceptable excipient, for inhibiting KRAS (e.g., KRAS protein having a Q61H, G12C, G12D, G12V, G12S, G12A, G12R, or G13D mutation, or wild-type KRAS) in a subject in need thereof.

The present disclosure also provides the use of a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, II, II′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof, or a pharmaceutical composition comprising any of the foregoing compounds and a pharmaceutically acceptable excipient, in the manufacture of a medicament for inhibiting KRAS (e.g., KRAS protein having a Q61H, G12C, G12D, G12V, G12S, G12A, G12R, or G13D mutation, or wild-type KRAS) in a subject in need thereof.

The present disclosure also provides a method comprising administering a therapeutically effective amount of a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IG, IIG1, HH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, III, III′, III-a, IIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof to a subject (e.g., patient) (e.g., a subject in need thereof), thereby ameliorating, reducing, eliminating, ceasing, delaying the progression of, or improving one or more symptoms of the subject, such as one or more symptoms of a discase, disorder, or condition (e.g., a cancer). In some embodiments, the subject has a cancer characterized by a mutant KRAS (e.g., KRAS protein having a Q61H, G12C, G12D, G12V, G12S, G12A, G12R, or G13D mutation, or wild-type KRAS).

In some embodiments, administering a therapeutically effective amount of a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, III, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof, slows or prevents growth of a tumor. In some embodiments, administering a therapeutically effective amount of a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, II, II′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof, results in shrinkage of a tumor (e.g., tumor regression). In some embodiments, administering a therapeutically effective amount of a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, II, II′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof, results in at least about 5%, 10%, 15%, 20%, 25%, 30%, 35%, 40%, 45%, 50% c, 55%, 60%, 65%, 70%, 75%, 80%, 85%, 90%, 95%, 96%, 97%, 98%, or 99% regression of a tumor, such as for a period of one or more weeks (e.g., at least about 1, 2, 3, 4, 5, 6, 7, 8, 9, 10, 11, 12, or more weeks), a period of one or more months (e.g., at least about 1, 2, 3, 4, 5, 6, 7, 8, 9, 10, 11, 12, or more months), or a period of one or more years (e.g., at least about 1, 2, 3, or more years). In some embodiments, administering a therapeutically effective amount of a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, II, II′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof, stabilizes a tumor. In some embodiments, administering a therapeutically effective amount of a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IX1, IIY, IIY1, IIZ, IIZ1, IIAA, IIAA1, III, III′, III-a, IIIA, IIIA1, IIIB, IIIC, IIIC1, IIID, IIIE, IIIF, IIIG, IIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, zwitterionic form, or stereoisomer(s) thereof, stabilizes a tumor for a period of one or more weeks (e.g., at least about 1, 2, 3, 4, 5, 6, 7, 8, 9, 10, 11, 12, or more weeks), a period of one or more months (e.g., at least about 1, 2, 3, 4, 5, 6, 7, 8, 9, 10, 11, 12, or more months), or a period of one or more years (e.g., at least about 1, 2, 3, or more years). In some embodiments, the subject has a cancer characterized by a mutant KRAS (e.g., KRAS protein having a Q61H, G12C, G12D, G12V, G12S, G12A, G12R, or G13D mutation, or wild-type KRAS).

In some embodiments of any of the methods, uses, and medicaments provided herein, the disease, disorder, or condition is a cancer. In some embodiments of any of the methods, uses, and medicaments provided herein, the cancer is pancreatic cancer (e.g., pancreatic ductal adenocarcinoma), lung cancer (e.g., non-small cell lung cancer), colorectal cancer (CRC), endometrial cancer, uterine carcinosarcoma, Ewing sarcoma, osteosarcoma, Rhabdomyosarcoma, adrenocortical carcinoma, neuroblastoma, Wilm tumor, retinoblastoma, skin cancer, breast cancer, prostate cancer, head and neck cancer, or ovarian cancer. In some embodiments, the cancer is pancreatic cancer (e.g., pancreatic ductal adenocarcinoma), lung cancer (e.g., non-small cell lung cancer adenocarcinoma), or colorectal cancer (CRC). In some embodiments, the cancer is pancreatic cancer (e.g., pancreatic ductal adenocarcinoma). In some embodiments, the cancer is lung cancer (e.g., non-small cell lung cancer adenocarcinoma). In some embodiments, the cancer is colorectal cancer (CRC). In some embodiments, the cancer is or comprises a solid tumor.

In some embodiments of any of the methods, uses, and medicaments provided herein, the disease, disorder, or condition is related to KRAS, such as a disorder associated with a mutation of KRAS or dysregulation of KRAS. In some embodiments, the disease, disorder, or condition is related to the KRAS gene, such as a disease, disorder, or condition associated with a mutation of the KRAS gene or dysregulation of the KRAS gene. Mutation or dysregulation of KRAS or KRAS may include mutation or dysregulation of human K-Ras4a and/or human K-Ras4b. In some embodiments, the disease, disorder, or condition is related to the KRAS (e.g., human K-Ras4a or K-Ras4b) signaling pathway activity, such as a disease, disorder, or condition related to aberrant KRAS signaling pathway activity. In some embodiments, the disease, disorder, or condition is related to mutation or dysregulation of human K-Ras4b. In some embodiments, the disease, disorder, or condition is related to aberrant K-Ras4b signaling pathway activity. In some embodiments, the disease, disorder, or condition is related to mutation or dysregulation of human K-Ras4a. In some embodiments, the disease, disorder, or condition is related to aberrant K-Ras4a signaling pathway activity.

### Administration and Combination Therapy

The compounds provided herein (e.g., compounds of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IX1, IIY, IIY1, IZ, IIZ1, IIAA, IIAA1, III, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a) and forms thereof (e.g., salt, ester, tautomer, prodrug, zwitterionic form, stereoisomer(s), etc.), or compositions (e.g., pharmaceutical compositions) comprising the same, can be administered in various modes (e.g., orally, topically, or by injection). The amount of active ingredient (e.g., a compound provided herein in any suitable form thereof) administered to a subject (e.g., patient) will be the responsibility of an attendant medical provider. The specific dose level for a given subject (e.g., patient) will depend on a variety of factors including, for example, the activity of the active ingredient administered; the physical attributes of the subject (e.g., age, weight, height, body mass index, general health, co-morbidities, sex, etc.); other characteristics of the subject (e.g., diet, level of exercise, national origin, ethnicity, etc.); time of administration; route of administration; rate of excretion; drug combination; the disease, disorder, or condition being treated; and the severity of the disease, disorder, or condition being treated.

In some embodiments, a compound provided herein (e.g., a compound of any one of Formulas I, I-a, IA, IA1, IA2, IB, IB1, IB2, IC, IC1. IC2, ID, ID1, ID2, IE, IE1, IE2, IF, IF1, IF2, II, II′, II-a, IIA, IIA1, IIB, IIB1, IIC, IIC1, IID, IID1, IIE, IIIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, IIH, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a form thereof (e.g., salt, ester, tautomer, prodrug, zwitterionic form, stereoisomer(s), etc.) is administered in combination with an additional agent, such as an additional therapeutic agent. For example, if a subject experiences a side effect such as hypertension upon receiving a compound provided herein, or a form thereof, it may be appropriate to administer an additional agent that is effective in managing the side effect, such as an anti-hypertensive agent. In another example, the therapeutic effectiveness of a compound provided herein (e.g., a compound of any one of Formulas I, I-a, IA, IA1, IA2, IB, IB, IB2, IC, IC1, IC2, ID, ID1, ID2, IE, IE1, IE2, IF, IF1, IF2, IT, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU, IIV, IIV1, IIW, IIW1, IX, IIX1, IIY, IIY1, IIZ, IIZ1, IIAA, IIAA1, III, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, V, IV-a, IVA, IVB, IVC, V, V-a, VA, VB, VC, VI, and VI-a), or a form thereof, may be enhanced by administration of an adjuvant, which adjuvant may itself have only minimal therapeutic benefit, but in combination with another therapeutic agent may provide an enhanced overall therapeutic benefit to a subject. In a further example, the therapeutic benefit of a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, I′, II-a, IIA, IIA1, IIB, IIB1, IIC, IIC1, ID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, III, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a form thereof, may be enhanced by administration of the compound, or a form thereof, and an additional agent (which may comprise an additional therapeutic regimen) that also provides a therapeutic benefit. For example, a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, III, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a form thereof, may be administered in combination with an additional agent that may be effective in the treatment of a disease, disorder, or condition such as a cancer. Generally, the combination of a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IB1, IIC, IIC1, IID, IID1, IIE, IIE1, IIF, IIF1, IIG, IIG1, IIH, IIH1, IIJ, IIJ1, IIK, IIK1, IIL, IIL1, IM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, IIIB, IIIC, III-a, IIIA, IIIA1, IIIB, IIIC, IIIC, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a form thereof, and one or more additional agents (e.g., therapeutic agents) may enhance the overall benefit experienced by the subject upon either component individually. In some embodiments, the effect may be additive. In some embodiments, the effect may be synergistic.

In some embodiments, a compound provided herein (e.g., a compound of any one of Formulas I, I-a, IA, IA1, IA2, IB, IB1, IB2, IC, IC1. IC2, ID, ID1, ID2, IE, IE1, IE2, IF, IF1, IF2, II, II′, II-a, IIA, IIA1, IIB, IIB1, IIC, IIC1, IID, IID1, IIE, IIIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, IIH, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a form thereof (e.g., salt, ester, tautomer, prodrug, zwitterionic form, stereoisomer(s), etc.) is administered in combination with an anti-cancer agent (e.g., chemotherapeutic agent). An anti-cancer agent may be, for example, an alkylating agent, an antimitotic, a checkpoint inhibitor, an anti-metabolite, a plant alkaloid, a terpenoid, a cytotoxic agent, an antibiotic, a topoisomerase inhibitor, an aromatase inhibitor, an angiogenesis inhibitor, an anti-steroid, an anti-androgen, an mTOR inhibitor, monoclonal antibodies, or a tyrosine kinase inhibitor. An alkylating agent may be, for example, armustine, chlorambucil (LEUKERAN), cisplatin (PLATIN), carboplatin (PARAPLATIN), oxaliplatin (ELOXATIN), streptozocin (ZANOSAR), busulfan (MYLERAN), dacarbazine, ifosfamide, lomustine (CCNU), melphalan (ALKERAN), procarbazine (MATULAN), temozolomide (TEMODAR), thiotepa, or cyclophosphamide (ENDOXAN). An anti-metabolite may be, for example, cladribine (LEUSTATIN), mercaptopurine (PURINETHOL), thioguanine, pentostatin (NIPENT), cytosine arabinoside (cytarabine, ARA-C), gemcitabine (GEMZAR), fluorouracil (5-FU, CARAC), capecitabine (XELODA), leucovorin (FUSILEY), methotrexate (RHEUMATREX), or raltitrexed. An antimitotic may be, for example, a taxane such as docetaxel (TAXITERE) or paclitaxcel (ABRAXANE, TAXOL), or a vinca alkaloid such as vincristine (ONCOVIN), vinblastine, vindesine, or vinorelbine (NAVELBINE). A checkpoint inhibitor may be an anti-PD-1 or anti-PD-L1 antibody such as pembrolizumab (KEYTRUDA), nivolumab (OPDIVO), MED14736, or MPDL3280A; anti-CTLA-4 antibody ipilimumab (YERVOY); or an agent that targets LAG3 (lymphocyte activation gene 3 protein), KIR (killer cell immunoglobulin-like receptor), 4-1BB (tumor necrosis factor receptor superfamily member 9), IIM3 (T-cell immunoglobulin and mucin-domain containing-3), or OX40 (tumor necrosis factor receptor superfamily member 4). A topoisomerase inhibitor may be, for example, camptothecin (CTP), irinotecan (CAMPTOSAR), topotecan (HYCAMTIN), teniposide (VUMON), or etoposide (EPOSIN). A cytotoxic antibiotic may be, for example, actinomycin D (dactinomycin, COSMEGEN), bleomycin (BLENOXANE) doxorubicin (ADRIAMYCIN), daunorubicin (CERUBIDINE), epirubicin (ELLENCE), fludarabine (FLUDARA), idarubicin, mitomycin (MITOSOL), mitoxantrone (NOYANTRONE), or plicamycin. An aromatase inhibitor may be, for example, aminoglutethimide, anastrozole (ARIMIDEX), letrozole (FEMARA), vorozole (RIYIZOR), or exemestane (AROMASIN). An angiogenesis inhibitor may be, for example, genistein, sunitinib (SUTENT), or bevacizumab (AYASTIN). An anti-steroid or anti-androgen may be, for example, aminoglutethimide (CYTADREN), bicalutamide (CASODEX), cyproterone, flutamide (EULEXIN), or nilutamide (NILANDRON). A tyrosine kinase inhibitor may be, for example, imatinib (GLEEVEC), erlotinib (TARCEVA), afatinib (GILOTRIF), lapatinib (TYKERB), sorafenib (NEXAVAR), or axitinib (INLYTA). An mTOR inhibitor may be, for example, everolimus, temsirolimus (TORISEL), or sirolimus. Monoclonal antibody may be, for example, trastuzumab (HERCEPTIN) or rituximab (RITUXAN). Additional examples of agents that may be useful in combination with a compound provided herein, or an alternative form thereof, include, but are not limited to, amsacrine; Bacillus Calmette-Guerin (B—C-G) vaccine; buserelin (ETILAMIDE); chloroquine (ARALEN); clodronate, pamidronate, and other bisphosphonates; colchicine; demethoxyviridin; dichloroacetate; estramustine; filgrastim (NEUPOGEN); fludrocortisone (FLORINEF); goserelin (ZOLADEX); interferon; leucovorin; leuprolide (LUPRON); levamisole; lonidamine; mesna: metformin; mitotane (o,r′-DDD, LYSODREN): nocodazole; octreotide (SANDOSTATIN); perifosine; porfimer (particularly in combination with photo- and radiotherapy); suramin: tamoxifen; titanocene dichloride; tretinoin; anabolic steroids such as fluoxymesterone (HALOTESTIN); estrogens such as estradiol, diethylstilbestrol (DES), and dienestrol; progestins such as medroxyprogesterone acetate (MPA) and megestrol; and testosterone.

Two or more therapeutic agents, one of which is a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, IIH, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IIIV, IV-a, IVA, IVB, IVC, V, V-a, VA, VB, VC, VI, and VI-a) or a form thereof, may be administered in any order or may be administered simultaneously. If administered simultaneously, the multiple therapeutic agents may be provided in a single, unified form, or in multiple forms (such as, for example, as a single pill or as two separate pills). One of the therapeutic agents may be given in multiple doses, or both may be given as multiple doses. If not administered simultaneously, the timing between the multiple doses may be any duration of time ranging from a few minutes to four weeks.

Accordingly, in another aspect, the present disclosure provides a method for treating a disease, disorder, or condition (e.g., a cancer) in a subject (e.g., a human or animal subject) in need of such treatment comprising administering to the subject an amount of a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, IIH, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IIIV, IV-a, IVA, IVB, IVC, V, V-a, VA, VB, VC, VI, and VI-a) or a form thereof (e.g., salt, ester, tautomer, prodrug, zwitterionic form, stereoisomer(s), etc.), in combination with at least one additional agent for the treatment of the disease, disorder, or condition. In a related aspect, the present disclosure provides a composition (e.g., pharmaceutical composition) comprising a compound provided herein (e.g., a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, IIH, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IIIV, IV-a, IVA, IVB, IVC, V, V-a, VA, VB, VC, VI, and VI-a), or a form thereof (e.g., salt, ester, tautomer, prodrug, zwitterionic form, stereoisomer(s), etc.), and at least one additional agent for use in the treatment of a disease, disorder, or condition (e.g., a cancer).

In some embodiments, a method provided herein is used to treat a disease, disorder, or condition a compound of any one of Formulas I, I-a, IA, IA1, A2, IB, IB1, IB2, IC, IC1, IC2, ID, ID1, ID2, IE, IE1, IE2, IF, IF1, IF2, II, II′, II-a, IIA, IA1, IIB, IIB1, IIC, IIC1, IID, IID1, IIE, IIE1, IIF, IIF1, IIG, IIG1, IIH, IIH1, IIJ, IIJ1, IIK, IIK1, IIL, IIL1, IIM, IIM1, IIN, IIN1, IIP, IIP1, IIQ, IIQ1, IIR, IIR1, IIS, IIS1, IIT, IIT1, IIU, IIU1, IIV, IIV1, IIW, IIW1, IIX, IIX1, IIY, IIY1, IIZ, IIZ1, IIAA, IIAA1, III, III′, III-a, IIIA, IIIA1, IIIB, IIIC, IIIC, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a), or a salt, ester, tautomer, prodrug, wherein the disease, disorder, or condition is a cancer that has developed a resistance to one or more chemotherapeutic drugs and/or ionizing radiation. In some embodiments, a method provided herein is used to treat a disease, disorder, or condition (e.g., a cancer) comprising administering to a subject in need thereof a therapeutically effective amount of a compound of any one of Formulas I, I-a, IA, IA1, IA2, IB, IB1, IB2, IC, IC1, IC2, ID, ID1, ID2, IE, IE1, IE2, IF, IF1, IF2, II, II′, II-a, IIA, IIA1, IIB, IIB1, IIC, IIC1, IID, ID1, IIE, IIE1, IIF, IIF1, IIG, IIG1, IIK, IIH1, IIJ, IIJ1, IIK, IIK, IIJ, IIJ1, IIM, IIM1, IIN, IIN1, IIP, IIP1, IIQ, IIQ1, IIR, IIR1, IIS, IIS1, IIT, IIT1, IIU, IIU1, IIV, IV1, IIW, IIW1, IIX, IIX1, IIY, IIY1, IIZ, IIZ1, IIAA, IIAA1, III, III′, III-a, IIIA, IIIA1, IIIB, IIIC, IIIC1, IIID, IIIE, IIIF, IIIG, IIIH, IIIJ, IIIK, IIIL, IIIM, IIIN, IIIP, IIIQ, IIIR, IIIS, IIIT, IV, IV-a, IVA, IVB, IVC, V, V-a, VA, VB, VC, VI, and VI-a or a pharmaceutically acceptable salt thereof, in combination with an additional agent wherein the disease, disorder, or condition is a cancer that has developed a resistance to one or more chemotherapeutic drugs and/or ionizing radiation.

The compounds, compositions, and methods disclosed herein are useful for the treatment of a disease, disorder, or condition, such as a cancer. In certain embodiments, the disease is one of dysregulated cellular proliferation, including cancer. The cancer may be hormone-dependent or hormone-resistant, such as in the case of breast cancers. In certain embodiments, the cancer is or comprises a solid tumor. In other embodiments, the cancer is a lymphoma or leukemia. In certain embodiments, the cancer is a drug resistant phenotype of a cancer disclosed herein or otherwise known. Tumor invasion, tumor growth, tumor metastasis, and angiogenesis may also be treated using the compositions and methods disclosed herein. In some embodiments, the compounds, compositions, and methods provided herein are also useful in the treatment of precancerous neoplasias.

Cancers that may be treated by the methods disclosed herein include, but are not limited to, pancreatic cancer, colon cancer, rectal cancer, colorectal cancer, breast cancer, ovarian cancer, endometrial cancer, lung cancer, and prostate cancer; cancers of the oral cavity and pharynx (lip, tongue, mouth, larynx, pharynx), esophagus, stomach, small intestine, large intestine, colon, rectum, liver and biliary passages; pancreas, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; and thyroid and other endocrine glands. The term “cancer” also encompasses cancers that do not necessarily form solid tumors, including Hodgkin's disease, non-Hodgkin's lymphomas, multiple myeloma, and hematopoietic malignancies including leukemias (Chronic Lymphocytic Leukemia (CLL), Acute Lymphocytic Leukemia (ALL), Chronic Myelogenous Leukemia (CML), Acute Myelogenous Leukemia (AML),) and lymphomas including lymphocytic, granulocytic and monocytic lymphomas. Additional types of cancers which may be treated using the compounds and methods provided herein include, but are not limited to, adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, head and neck cancer, hemangioblastoma, hepatocellular carcinoma, hepatoma. Kaposi's sarcoma, large cell carcinoma, leiomyosarcoma, leukemias, liposarcoma, lymphatic system cancer, lymphomas, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, paraganglioma, parathyroid tumors, pheochromocytoma, pinealoma, plasmacytomas, retinoblastoma, rhabdomyosarcoma, sebaceous gland carcinoma, seminoma, skin cancers, melanoma, small cell lung carcinoma, non-small cell lung carcinoma, squamous cell carcinoma, sweat gland carcinoma, synovioma, thyroid cancer, uveal melanoma, and Wilm's tumor. Additional diseases and disorders that may be treated by the methods disclosed herein include, but are not limited to, diseases or disorders related to KRAS, such as diseases or disorders associated with a mutation of KRAS (e.g., KRAS protein having a Q61H, G12C, G12D, G12V, G12S, G12A, G12R, or G13D mutation, or wild-type KRAS) or dysregulation of KRAS, and discases or disorders related to the KRAS gene, such as diseases or disorders associated with a mutation of the KRAS gene or dysregulation of the KRAS gene.

In some embodiments, the compounds, compositions, and methods provided herein are useful in the prevention and/or reduction of tumor invasion, growth, and/or metastasis.

The compounds, compositions, and methods provided herein may be useful in the treatment of humans as well as in the veterinary treatment of non-human animals including companion animals, exotic animals, and farm animals (e.g., as described herein), including mammals, rodents, and the like. For example, the compounds, compositions, and methods provided herein may be useful in the treatment of horses, dogs, or cats.

### EXEMPLARY EMBODIMENTS

The following numbered embodiments, while non-limiting, are exemplary of certain aspects of the present disclosure:
A1. A compound represented by Formula I:

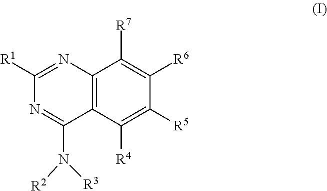

or a salt (e.g., pharmaceutically acceptable salt) thereof, wherein:
R¹ is selected from —OR⁸ and

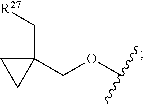

R² is selected from H and C₁₋₆alkyl, wherein any C₁₋₆ alkyl is unsubstituted or is substituted with one or more R¹³;
R³ is selected from a 3-11 membered carbocycle that is unsubstituted or substituted with one or more R¹⁰;
R⁴ is H;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁶ is a bicyclic heteroaryl substituted with one or more R¹⁵;
R⁷ is selected from halogen, —CN, and H;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹⁰ is independently selected from —OR¹², —C(O)(C₁₋₆alkylene)CN, —C(O(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹⁴)₂, —CN, and C₁₋₆alkyl, wherein any C₁₋₆ alkyl is unsubstituted or substituted with one or more R¹³;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R¹³;
each R²⁸ is independently selected from C₁₋₆alkyl and halogen; and
R^{a} and R^{b} are each independently selected from halogen, C₁₋₆ alkyl, —OR¹², and H, wherein any C₁₋₆alkyl is unsubstituted or is substituted with one or more R¹³.
A2. The compound of embodiment A1, wherein R³ is a 3-6 membered carbocycle that is unsubstituted or is substituted with one or more R¹⁰.
A3. The compound of embodiment A2, wherein R³ is a 3-6 membered carbocycle that is substituted with 1-4 R¹⁰.
A4. The compound of embodiment A2, wherein R³ is a cyclopropane that is substituted with 0-4 R¹⁰.
A5. The compound of embodiment A2, wherein R³ is a cyclobutane that is substituted with 0-4 R¹⁰.
A6. The compound of embodiment A2, wherein R³ is a cyclopentane that is substituted with 0-4 R¹¹.
A7. The compound of embodiment A2, wherein R³ is a cyclohexane that is substituted with 0-4 R¹⁰.
A8. The compound of any one of embodiments A1-A3, wherein R³ is a spirocycle that is unsubstituted or is substituted with one or more R¹⁰
A9. The compound of any one of embodiments A1-A8, wherein R³ is substituted with one or more R¹⁰, wherein at least one R¹⁰ is selected from —OR¹² and a C₁₋₆alkyl substituted with —OH.
A10. The compound of any one of embodiments A1-A9, wherein R³ is substituted with one or more R¹⁰, wherein at least one R¹⁰ is an unsubstituted C₁₋₆alkyl.
A11. The compound of any one of embodiments A1-A10, wherein the compound is a compound according to Formula IA, Formula IB, or Formula IC:

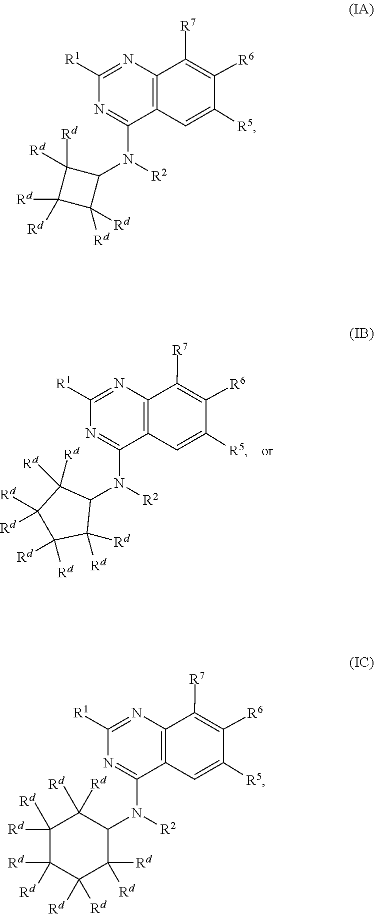

or a salt (e.g., pharmaceutically acceptable salt) thereof, wherein:
each R^{d} is independently selected from H, —OR¹², —C(O)(C₁₋₆alkylene)CN, —C(O)(C₁₋₆ alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰.
A12. The compound of embodiment A11, wherein the compound is a compound according to Formula IA, or a salt (e.g., pharmaceutically acceptable salt) thereof.
A13. The compound of embodiment A11, wherein the compound is a compound according to Formula IB, or a salt (e.g., pharmaceutically acceptable salt) thereof.
A14. The compound of embodiment A11, wherein the compound is a compound according to Formula IC, or a salt (e.g., pharmaceutically acceptable salt) thereof.
A15. The compound of any one of embodiments A11-A14, wherein at least one R^{d} is selected from —OR¹⁴, —C(O)C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰.
A16. The compound of embodiment A15, wherein at least one R^{d} is selected from —OR¹² and C₁₋₆alkyl, wherein any C₁₋₆alkyl is unsubstituted or substituted with one or more R²⁰.
A17. The compound of any one of embodiments A1-A16, wherein R⁶ is selected from:

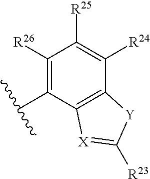

wherein:
   X is selected from N and C—CN;
   Y is selected from O and S;
   R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
   R²⁴, R^{2S}, and R²⁶ are independently selected from H, halogen, and C₁₋₆alkyl, wherein any C₁₋₆alkyl is unsubstituted or substituted with one or more R¹³.
A18. The compound of any one of embodiments A1-A17, wherein R⁶ is selected from:

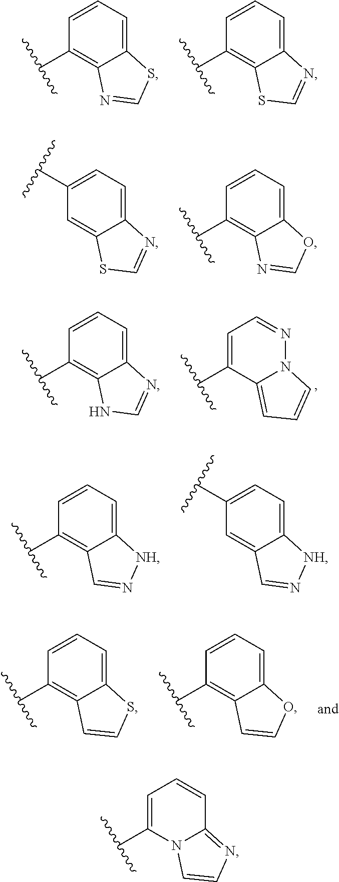

any of which is substituted with one or more R¹⁵.
A19. The compound of any one of embodiments A1-A18, wherein R⁶ is selected from:

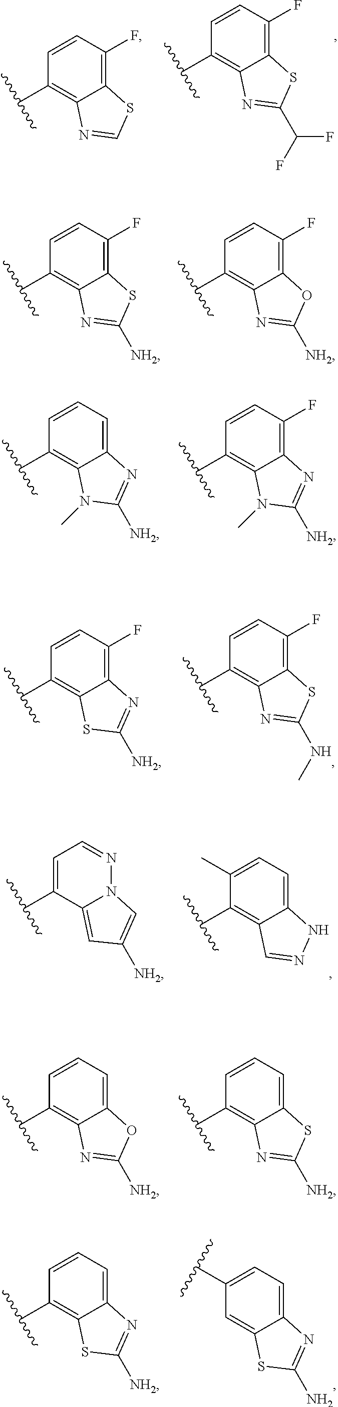

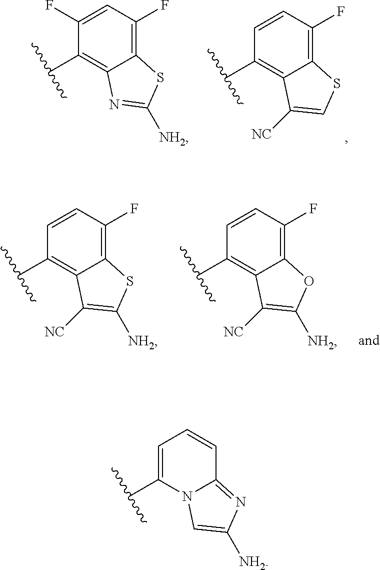 [SMILES: *c1c(F)cc(F)c2sc(N)nc12 ; *c1ccc(F)c2scc(C#N)c12 ; *c1ccc(F)c2sc(N)c(C#N)c12 ; *c1ccc(F)c2oc(N)c(C#N)c12 ; *c1cccc2nc(N)cn12]

A20. The compound of anyone of embodiments A1-A19, wherein R⁶ is selected from:

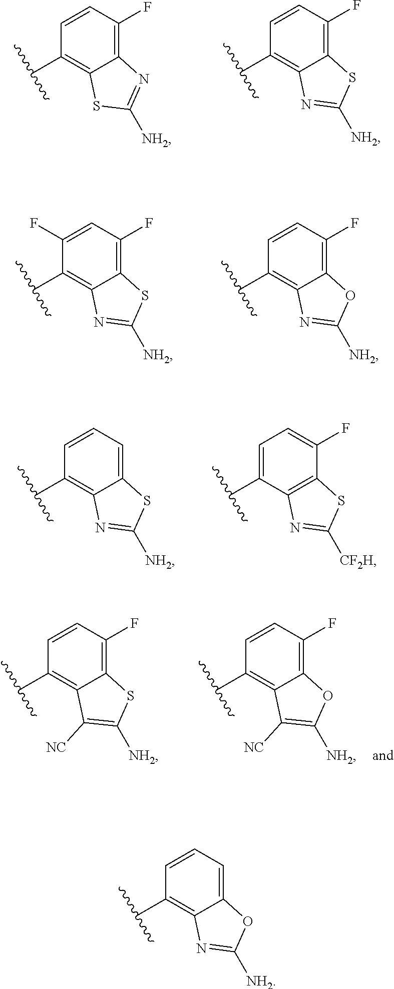

A21. The compound of any one of embodiments A1-A20, wherein the compound is a compound according to Formula IA1, IB1, or IC1:

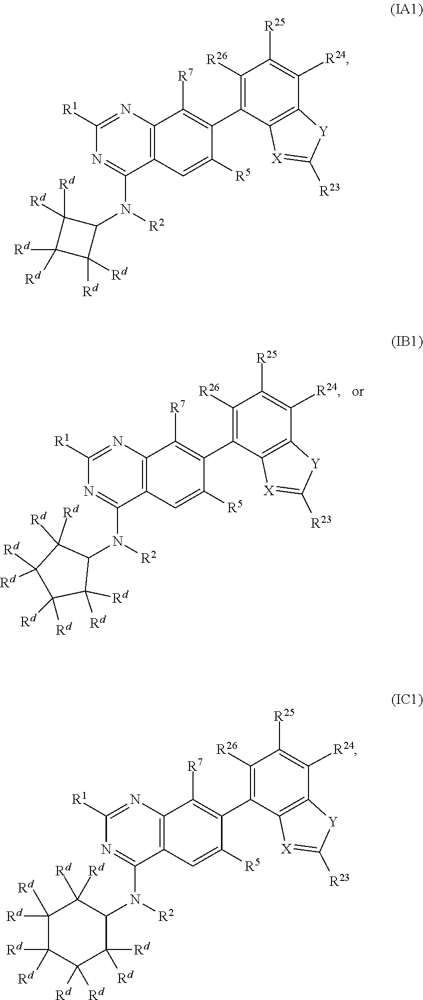

or a salt (e.g., pharmaceutically acceptable salt) thereof, wherein:
each R^{d} is independently selected from H, —OR¹⁴, —C(O)(C₁₋₆alkylene)CN, —C(O)(C₁₋₆ alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆-alkyl, wherein any C₁₋₆alkyl is unsubstituted or substituted with one or more R¹³.
A22. The compound of embodiment A21, wherein the compound is a compound according to Formula IA1, or a salt (e.g., pharmaceutically acceptable salt) thereof.
A23. The compound of embodiment A21, wherein the compound is a compound according to Formula IB1, or a salt (e.g., pharmaceutically acceptable salt) thereof.
A24. The compound of embodiment A21, wherein the compound is a compound according to Formula IC1, or a salt (e.g., pharmaceutically acceptable salt) thereof.
A25. The compound of any one of embodiments A21-A24, wherein X is C—CN, Y is S, and R²³ is —N(R¹²)₂.
A26. The compound of any one of embodiments A21-A25, wherein one or more of R²⁴, R²⁵, and R²⁶ is a halogen (e.g., F).
A27. The compound of any one of embodiments A1-A26, wherein R¹ is selected from —OR⁸.
A28. The compound of embodiment A27, wherein R¹ is selected from:

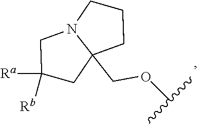

wherein R^{a} and R^{b} are each independently selected from halogen, C₁₋₆alkyl, —OR¹², and H, wherein any C₁₋₆ alkyl is unsubstituted or is substituted with one or more R¹³.
A29. The compound of embodiment A28, wherein R¹ is selected from:

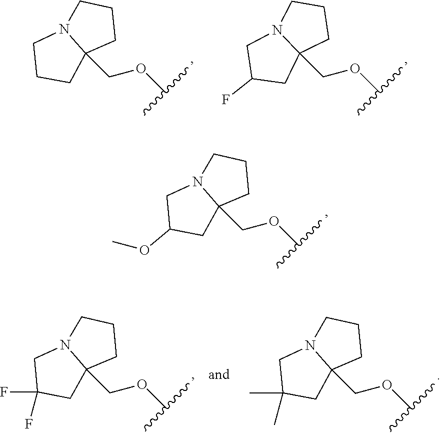 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

A30. The compound of embodiment A27, wherein R¹ is selected from:

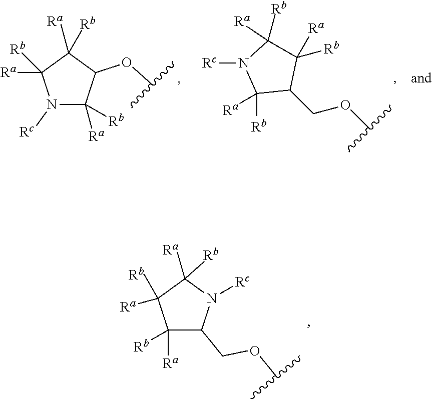

wherein each R^{a} and R^{b} is independently selected from halogen, C₁₋₆ alkyl, —OR¹², and H; and R^{c} is selected from C₁₋₆ alkyl, wherein the C₁₋₆ alkyl is unsubstituted or is substituted with one or more R¹³.
A31. The compound of embodiment A27, wherein R¹ is selected from:

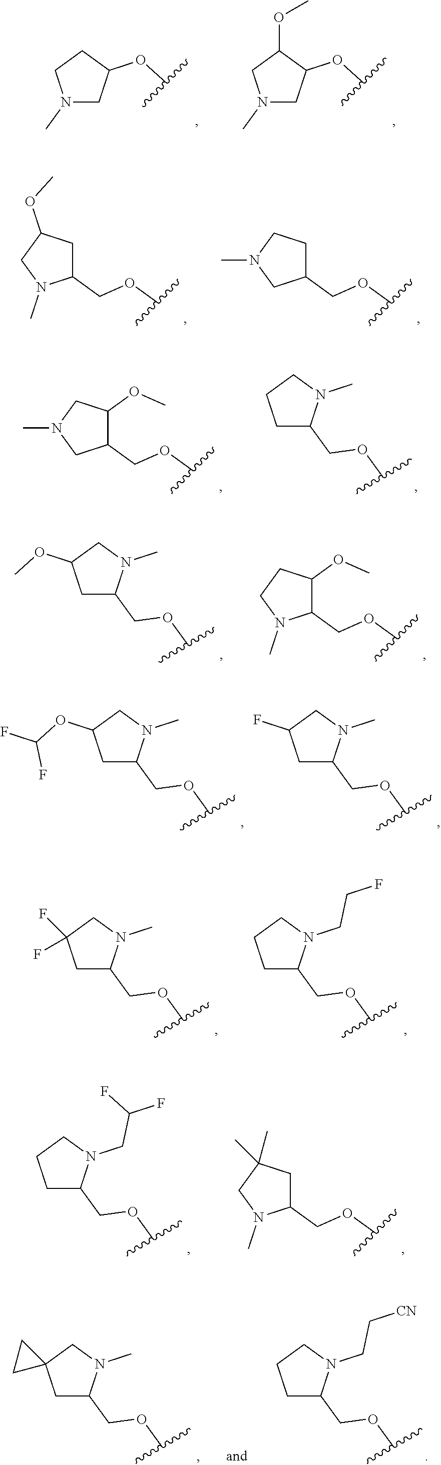

A32. The compound of any one of embodiments A1-A26, wherein R¹ is selected from

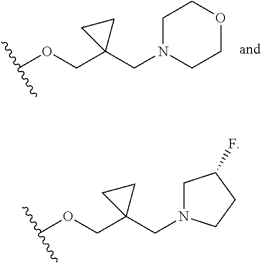 [SMILES: *OCC1(CN2CCOCC2)CC1 ; *OCC1(CN2CC[C@@H](F)C2)CC1]

A33. The compound of any one of embodiments A1-A32, wherein the compound is a compound according to Formula IA2, IB2, or IC2:

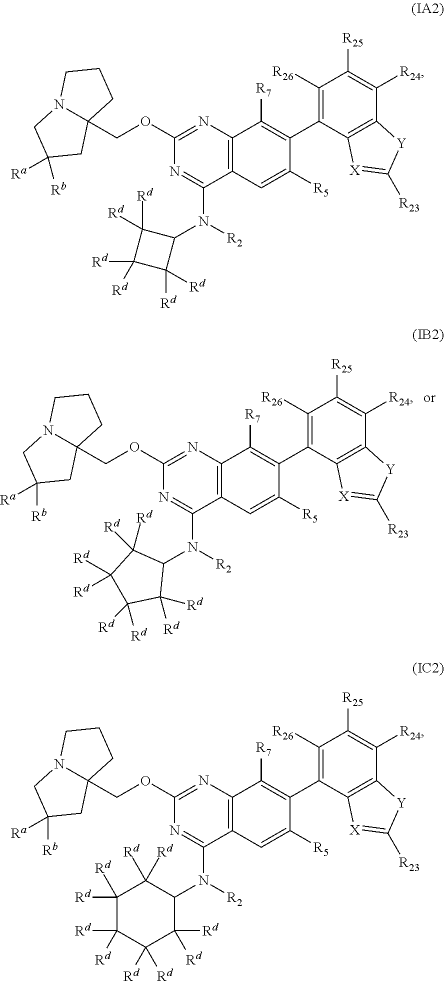

or a salt (e.g., pharmaceutically acceptable salt) thereof, wherein:
each R^{d} is independently selected from H, —OR¹², —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰;
X is selected from N and C—CN;
Y is selected from O and S;
R²³ is selected from —N(R¹²)₂, C₁₋₄alkyl, and C₁₋₄alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆alkyl, wherein any C₁₋₆alkyl is unsubstituted or substituted with one or more R¹³.
A34. The compound of embodiment A33, wherein the compound is a compound according to Formula IA2, or a salt (e.g., pharmaceutically acceptable salt) thereof.
A35. The compound of embodiment A33, wherein the compound is a compound according to Formula IB2, or a salt (e.g., pharmaceutically acceptable salt) thereof.
A36. The compound of embodiment A33, wherein the compound is a compound according to Formula IC2, or a salt (e.g., pharmaceutically acceptable salt) thereof.
A37. The compound of any one of embodiments A33-A36, wherein R^{a} is a halogen (e.g., F).
A38. The compound of any one of embodiments A33-A37, wherein R^{b} is H.
A39. The compound of any one of embodiments A21-A38, wherein X is C—CN, Y is S, and R²³ is —N(R¹²)₂.
A40. The compound of any one of embodiments A21-A39, wherein one or more of R²⁴, R²⁵, and R²⁶ is a halogen (e.g., F).
A41. The compound of any one of embodiments A1-A40, wherein R² is H.
A42. The compound of any one of embodiments A1-A40, wherein R² is selected from C₁₋₆alkyl that is unsubstituted or is substituted with one or more R¹³.
A43. The compound of embodiment A42, wherein R² is selected from C₁₋₂alkyl.
A44. The compound of any one of embodiments A1-A43, wherein R⁵ is H.
A45. The compound of any one of embodiments A1-A43, wherein R⁵ is a halogen (e.g., F or Cl).
A46. The compound of any one of embodiments A1-A43, wherein R⁵ is selected from C₁₋₆alkyl that is unsubstituted or substituted with one or more R¹³.
A47. The compound of embodiment A46, wherein R⁵ is selected from C₁₋₆alkyl that is substituted with one or more halogens or —CN.
A48. The compound of embodiment A47, wherein R⁵ is selected from —CF₂H, —CF₃, —CH₂CN, and —CH₂CH₃.
A49. The compound of embodiment A48, wherein R¹ is —CF₃.
A50. The compound of any one of embodiments A1-A49, wherein R⁷ is H.
A51. The compound of any one of embodiments A1-A49, wherein R⁷ is a halogen (e.g., F or Cl).
A52. The compound of any one of embodiments A1-A49, wherein R⁷ is —CN.
A53. A compound represented by Formula II′:

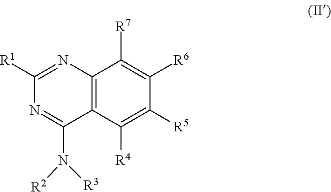

or a salt (e.g., pharmaceutically acceptable salt) thereof, wherein:
R¹ is selected from —OR⁸ and

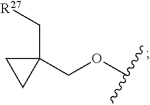

R² is selected from H and C₁₋₆alkyl, wherein any C₁₋₆ alkyl is unsubstituted or is substituted with one or more R¹³;
R³ is selected from a 4-6 membered heterocycle that is unsubstituted or substituted with one or more R¹⁰;
R⁴ is H;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁶ is a bicyclic heteroaryl substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹⁰ is independently selected from —OR¹⁴, ═O, —C(O)(C₁₋₆alkylene)CN, —C(O)(C₁₋₆alkylene)OH, —C(O)(C₁₋₆alkyl), —C(O)N(R¹⁴)₂, —S(O)₂(C₁₋₆alkyl), halogen, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹⁴)₂, —CN, and C₁₋₆alkyl, wherein any C₁₋₆ alkyl is unsubstituted or substituted with one or more R¹³;
each R²⁰ is independently selected from —OH, —OC₁₋₆alkyl, ═O, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen; and
R^{a} and R^{b} are each independently selected from halogen, C₁₋₆ alkyl, —OR¹², and H, wherein any C₁₋₆alkyl is unsubstituted or is substituted with one or more R¹³.
A54. A compound represented by Formula II:

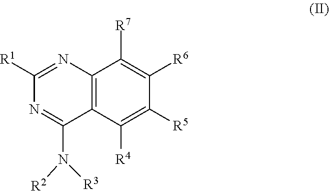

or a salt (e.g., pharmaceutically acceptable salt) thereof, wherein:
R¹ is selected from —OR⁸ and

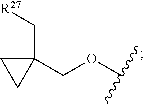

R² is selected from H and C₁₋₆alkyl, wherein any C₁₋₆alkyl is unsubstituted or is substituted with one or more R¹³;
R³ is selected from a 4-6 membered heterocycle that is unsubstituted or substituted with one or more R¹⁰, provided that when the heterocycle contains a nitrogen atom, the nitrogen atom is substituted with R¹⁰;
R⁴ is H;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁶ is a bicyclic heteroaryl substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹⁰ is independently selected from —OR¹⁴, —O, —C(O)(C₁₋₆alkylene)CN, —C(O)(C₁₋₆alkylene)OH, —C(O)(C₁₋₆alkyl), —C(O)N(R¹⁴); —S(O)₂(C₁₋₆alkyl), halogen, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹²)₂, —CN, and C₁₋₆alkyl, wherein any C₁₋₆alkyl is unsubstituted or substituted with one or more R¹³;
each R²⁰ is independently selected from —OH, —OC₁₋₆alkyl, ═O, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²¹;
each R²⁸ is independently selected from C₁₋₆alkyl and halogen; and
R^{a} and R^{b} are each independently selected from halogen, C₁₋₆ alkyl, —OR¹², and H, wherein any C₁₋₆alkyl is unsubstituted or is substituted with one or more R¹³.
A55. The compound of embodiment A53 or A54, wherein R³ is a 4-6 membered heterocycle that includes 1 heteroatom selected from O, S, and N.
A56. The compound of embodiment A55, wherein R³ is a 4-6 membered heterocycle that includes 1 heteroatom selected from O, S, and N, wherein the heterocycle is substituted with 1-4 R¹⁰, provided that when the heterocycle contains a nitrogen atom, the nitrogen atom is substituted with R¹⁰.
A57. The compound of any one of embodiments A53-A56, wherein R³ is a pyrrolidine that is substituted with 0-4 R¹⁰, provided that the nitrogen atom is substituted with R^{1Q}.
A58. The compound of any one of embodiments A53-A56, wherein R³ is an oxetane or thietane that is substituted with 0-4 R¹⁰.
A59. The compound of any one of embodiments A53-A56, wherein R³ is a tetrahydrofuran or tetrahydrothiopene that is substituted with 0-4 R¹⁰.
A60. The compound of any one of embodiments A53-A59, wherein R³ is a 4-6 membered heterocycle that is substituted with one or more R¹⁰, wherein at least one R¹⁰ is selected from —OR¹² and a C₁₋₆alkyl substituted with —OH.
A61. The compound of any one of embodiments A53-A60, wherein R³ is a 4-6 membered heterocycle that is substituted with one or more R¹⁰, wherein at least one R¹⁰ is an unsubstituted C₁₋₆alkyl.
A62. The compound of any one of embodiments A53-A61, wherein the compound is a compound according to Formula IIA, IIB, IIC, IID, IIIE, IIF, IIG, II, IIJ, or IIK:

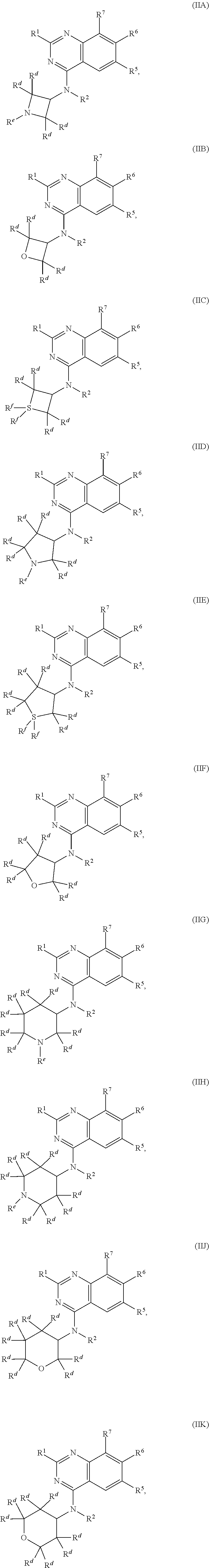

or a salt (e.g., pharmaceutically acceptable salt) thereof, wherein:
each R^{d} is independently selected from H, —OR¹², —O, —C(O)(C₁₋₆alkylene)CN, —C(O)(C₁₋₆ alkylene)OH, —C(O)(C₁₋₆alkyl), —C(O)N(R¹⁴)₂, —S(O)₂(C₁₋₆alkyl), halogen, and C₁₋₆alkyl, wherein any C₁₋₆alkyl is unsubstituted or substituted with one or more R²⁰;
R^{e}, if present, is selected from —C(O)(C₁₋₆alkyl)CN, —C(O)(C₁₋₆alkyl)OH, —C(O)(C₁₋₆alkyl), —C(O)N(R¹⁴)₂, —S(O)₂(C₁₋₆alkyl), and C₁₋₆alkyl, wherein any C₁₋₆alkyl is unsubstituted or substituted with one or more R²⁰; and
each R^{f} is optionally absent and, if present, is selected from ═O, —NH₂, —NHC₁₋₆alkyl, and —N(C₁₋₆alkyl)₂.
A63. The compound of embodiment A62, wherein the compound is a compound according to Formula IIA, or a salt (e.g., pharmaceutically acceptable salt) thereof.
A64. The compound of embodiment A62, wherein the compound is a compound according to Formula IIB, or a salt (e.g., pharmaceutically acceptable salt) thereof.
A65. The compound of embodiment A62, wherein the compound is a compound according to Formula IIC, or a salt (e.g., pharmaceutically acceptable salt) thereof.
A66. The compound of embodiment A62, wherein the compound is a compound according to Formula IID, or a salt (e.g., pharmaceutically acceptable salt) thereof.
A67. The compound of embodiment A62, wherein the compound is a compound according to Formula IIE, or a salt (e.g., pharmaceutically acceptable salt) thereof.
A68. The compound of embodiment A62, wherein the compound is a compound according to Formula IIF, or a salt (e.g., pharmaceutically acceptable salt) thereof.
A69. The compound of embodiment A62, wherein the compound is a compound according to Formula IIG, or a salt (e.g., pharmaceutically acceptable salt) thereof.
A70. The compound of embodiment A62, wherein the compound is a compound according to Formula IIH, or a salt (e.g., pharmaceutically acceptable salt) thereof.
A71. The compound of embodiment A62, wherein the compound is a compound according to Formula IIJ, or a salt (e.g., pharmaceutically acceptable salt) thereof.
A72. The compound of embodiment A62, wherein the compound is a compound according to Formula IIK, or a salt (e.g., pharmaceutically acceptable salt) thereof.
A73. The compound of any one of embodiments A62, A65, and A67, wherein the compound is a compound according to Formula IC or IIE, wherein each R^{f} is ═O.
A74. The compound of any one of embodiments A62, A63, A66, A69, and A70, wherein the compound is a compound according to Formula IIA IID, IIG, or IIH, wherein R^{e} is C₁₋₆alkyl that is unsubstituted or substituted with one or more R²⁰.
A75. The compound of any one of embodiments A53-A74, wherein R⁶ is selected from:

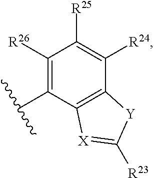

wherein:
   X is selected from N and C—CN;
   Y is selected from O and S;
   R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
   R²⁴, R²⁵, and R²⁶ are independently selected from H, halogen, and C₁₋₆alkyl, wherein any C₁₋₆alkyl is unsubstituted or substituted with one or more R¹³.
A76. The compound of any one of embodiments A53-A75, wherein R⁶ is selected from:

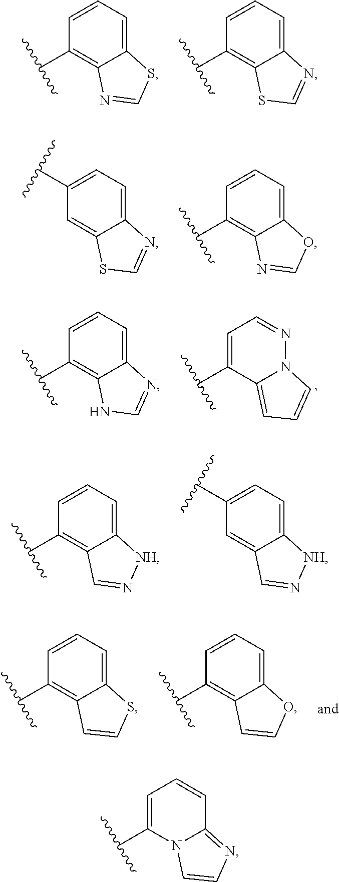

any of which is substituted with one or more R¹⁵.
A77. The compound of any one of embodiments A53-A75, wherein R⁶ is selected from:

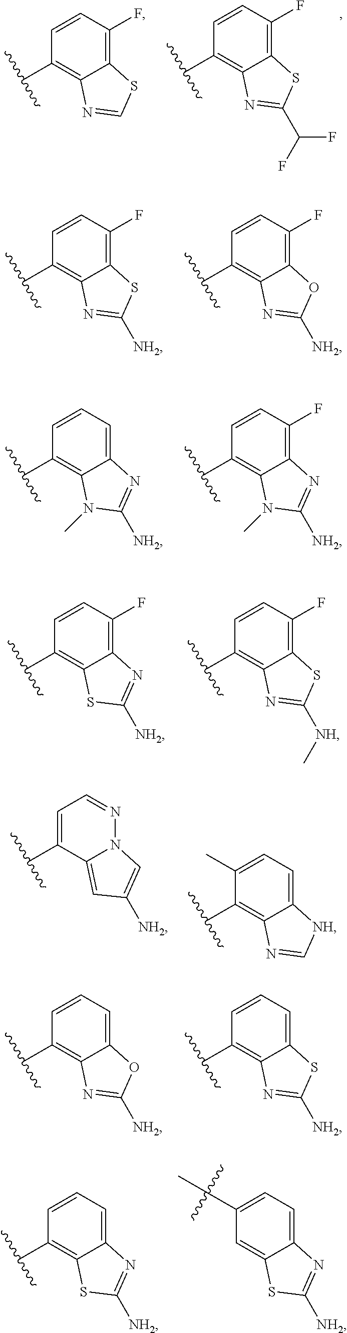

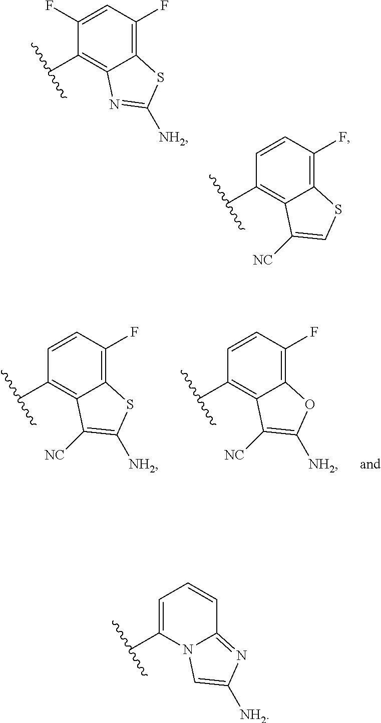 [SMILES: *c1c(F)cc(F)c2sc(N)nc12 ; *c1ccc(F)c2scc(C#N)c12 ; *c1ccc(F)c2sc(N)c(C#N)c12 ; *c1ccc(F)c2oc(N)c(C#N)c12 ; "and" ; *c1cccc2nc(N)cn12]

A78. The compound of any one of embodiments A53-A75, wherein R⁶ is selected from:

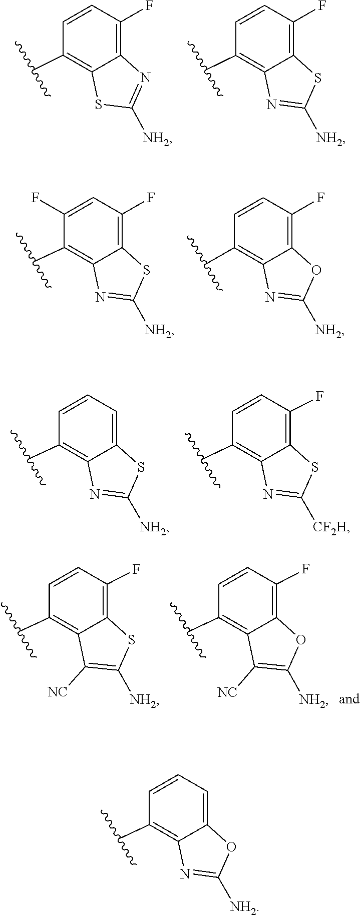

A79. The compound of any one of embodiments A53-A78, wherein the compound is a compound according to Formula IIA1, IIB1, IIC1, IID1, IIE1, IIF1, IIG1, IIH1, IIJ1, or IIK1:

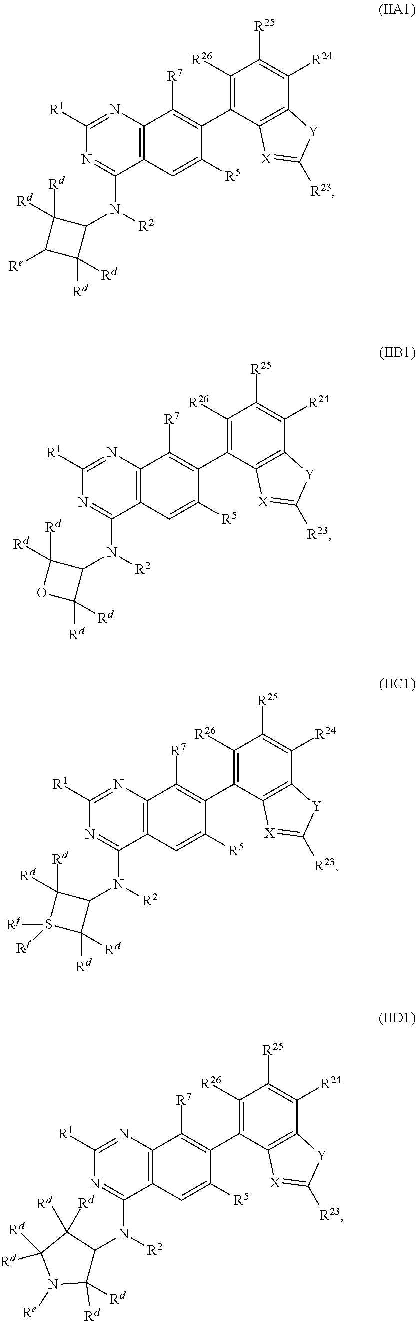

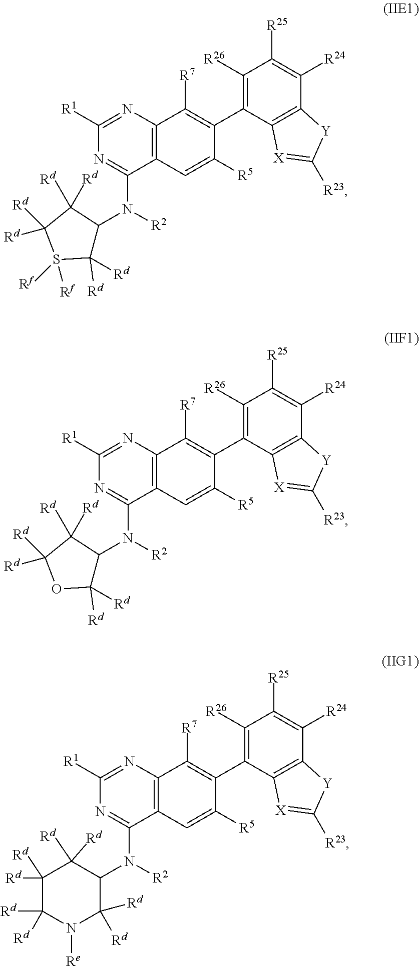

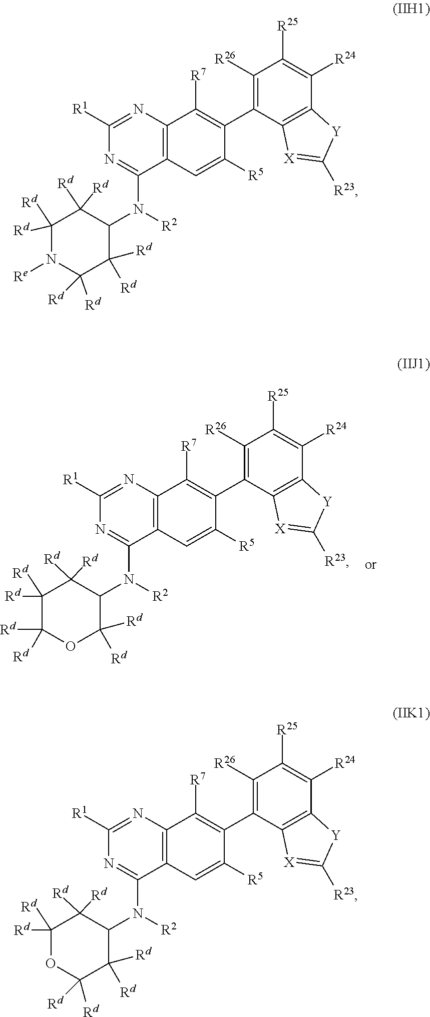

or a salt (e.g., pharmaceutically acceptable salt) thereof, wherein:
each R^{d} is independently selected from H, —OR¹², ═O, —C(O)(C₁₋₆alkylene)CN, —C(O)(C₁₋₆alkylene)OH, —C(O)(C₁₋₆alkyl), —C(O)N(R¹⁴)₂, —S(O)₂(C₁₋₆alkyl), halogen, and C₁₋₆alkyl, wherein any C₁₋₆alkyl is unsubstituted or substituted with one or more R²⁰;
R^{e}, if present, is selected from —C(O)(C₁₋₆alkylene)CN, —C(O)(C₁₋₆alkylene)OH, —C(O)(C₁₋₆alkyl), —C(O)N(R¹⁴)₂, —S(O)₂(C₁₋₆alkyl), and C₁₋₆alkyl, wherein any C₁₋₆alkyl is unsubstituted or substituted with one or more R²;
each R^{f} is optionally absent and, if present, is selected from ═O, —NH₂, —NHC₁₋₆alkyl, and —N(C₁₋₆alkyl)₂;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆alkyl, wherein any C₁₋₆alkyl is unsubstituted or substituted with one or more R¹³.
A80. The compound of embodiment A79, wherein the compound is a compound according to Formula IIA1, or a salt (e.g., pharmaceutically acceptable salt) thereof.
A81. The compound of embodiment A79, wherein the compound is a compound according to Formula IIB1, or a salt (e.g., pharmaceutically acceptable salt) thereof.
A82. The compound of embodiment A79, wherein the compound is a compound according to Formula IIC1, or a salt (e.g., pharmaceutically acceptable salt) thereof.
A83. The compound of embodiment A79, wherein the compound is a compound according to Formula IID1, or a salt (e.g., pharmaceutically acceptable salt) thereof.
A84. The compound of embodiment A79, wherein the compound is a compound according to Formula IIE1, or a salt (e.g., pharmaceutically acceptable salt) thereof.
A85. The compound of embodiment A79, wherein the compound is a compound according to Formula IIF1, or a salt (e.g., pharmaceutically acceptable salt) thereof.
A86. The compound of embodiment A79, wherein the compound is a compound according to Formula IIG1, or a salt (e.g., pharmaceutically acceptable salt) thereof.
A87. The compound of embodiment A79, wherein the compound is a compound according to Formula IIH1, or a salt (e.g., pharmaceutically acceptable salt) thereof.
A88. The compound of embodiment A79, wherein the compound is a compound according to Formula IIJ1, or a salt (e.g., pharmaceutically acceptable salt) thereof.
A89. The compound of embodiment A79, wherein the compound is a compound according to Formula IIK1, or a salt (e.g., pharmaceutically acceptable salt) thereof.
A90. The compound of any one of embodiments A79-A89, wherein X is C—CN, Y is S, and R^{c} is —N(R¹²)₂.
A91. The compound of any one of embodiments A79-A90, wherein one or more of R²⁵, R²⁶, and R²⁶ is a halogen (e.g., F).
A92. The compound of any one of embodiments A79, A82, A84, A90, and A91, wherein the compound is a compound according to Formula IIC1 or IIE1, wherein each R^{f} is ═O.
A93. The compound of any one of embodiments A79, A80, A83, A86, A87, A90, and A91, wherein the compound is a compound according to Formula IIA1, IID1, IIG1, or IIH1, wherein R is C₁₋₆alkyl that is unsubstituted or substituted with one or more R².
A94. The compound of any one of embodiments A62-A93, wherein each R is H.
A95. The compound of any one of embodiments A62-A93, wherein at least one R^{d} is selected from —OR¹² and a C₁₋₆alkyl substituted with —OH.
A96. The compound of any one of embodiments A53-A95, wherein R² is H.
A97. The compound of any one of embodiments A53-A95, wherein R² is selected from C₁₋₆alkyl that is unsubstituted or is substituted with one or more R¹³.
A98. The compound of embodiment A97, wherein R² is selected from C₁₋₂alkyl.
A99. The compound of any one of embodiments A53-A98, wherein R¹ is selected from —OR⁸.
A100. The compound of embodiment A99, wherein R¹ is selected from:

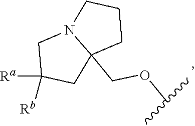

wherein R^{a} and R^{b} are each independently selected from halogen, C₁₋₆alkyl, —OR¹², and H, wherein any C_{1-b}alkyl is unsubstituted or is substituted with one or more R¹³.
A101. The compound of embodiment A100, wherein R¹ is selected from:

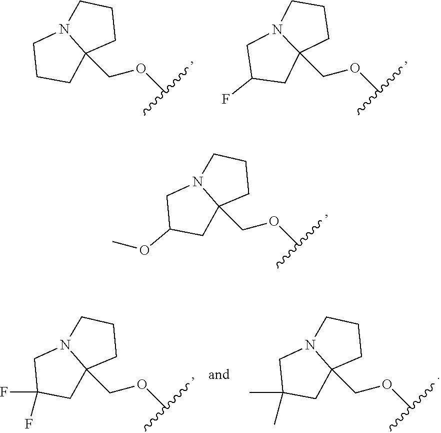 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

A102. The compound of embodiment A99, wherein R¹ is selected from:

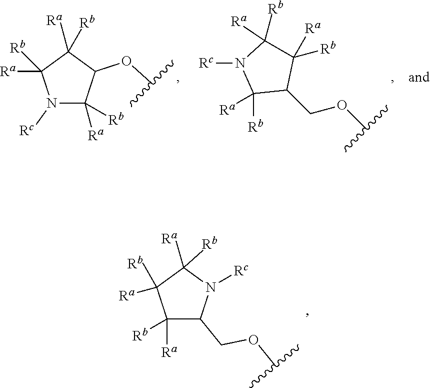

wherein each R^{a} and R^{b} is independently selected from halogen, C₁₋₆ alkyl, —OR¹², and H; and R^{c} is selected from C₁₋₆ alkyl, wherein the C₁₋₆ alkyl is unsubstituted or is substituted with one or more R¹³.
A103. The compound of embodiment A102, wherein R¹ is selected from:

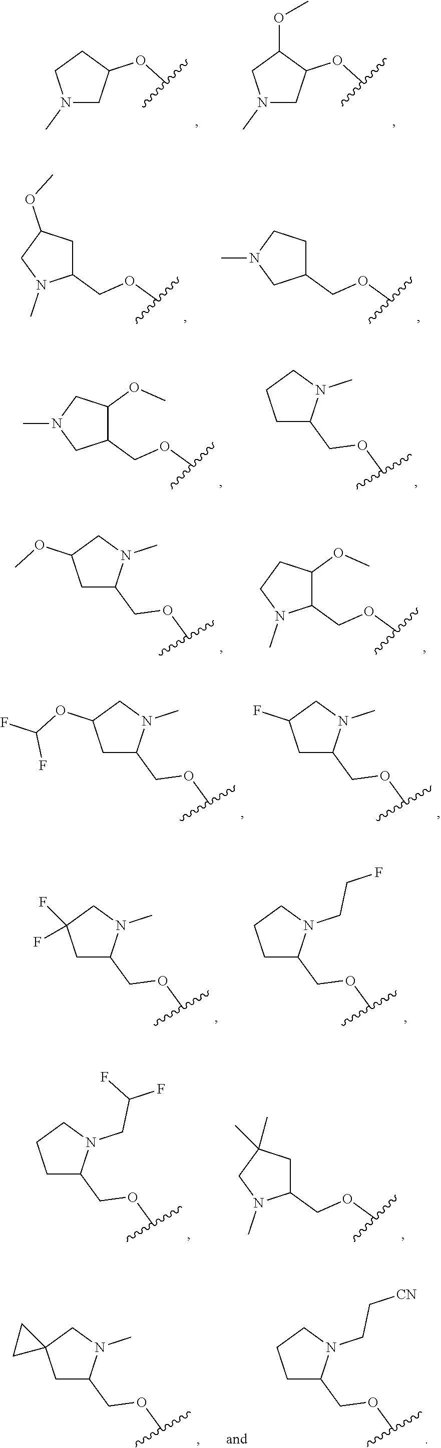

A104. The compound of any one of embodiments A53-A98, wherein R¹ is selected from

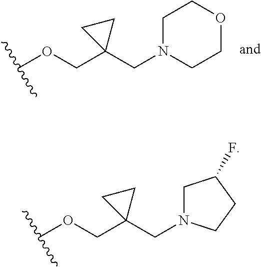 [SMILES: *OCC1(CN2CCOCC2)CC1 ; *OCC1(CN2CC[C@@H](F)C2)CC1]

A105. The compound of any one of embodiments A53-A104, wherein R¹ is H.
A106. The compound of any one of embodiments A53-A104, wherein R¹ is a halogen (e.g., F or Cl).
A107. The compound of any one of embodiments A53-A104, wherein R¹ is selected from C₁₋₆alkyl that is unsubstituted or substituted with one or more R¹³.
A108. The compound of embodiment A107, wherein R⁵ is selected from C₁₋₆alkyl that is substituted with one or more halogens or —CN.
A109. The compound of embodiment A108, wherein R¹ is selected from —CF₂H, —CF₃, —CH₂CN, and —CH₂CH₃.
A110. The compound of embodiment A109, wherein R⁵ is —CF₃.
AIII. The compound of any one of embodiments A53-A110, wherein the compound is a not a compound included in Table 1.
A112. A compound represented by Formula III′:

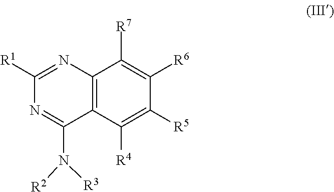

or a salt (e.g., pharmaceutically acceptable salt) thereof, wherein:
R¹ is selected from —OR⁸ and

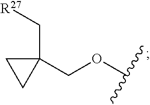

R² and R³, together with the nitrogen atom to which they are attached, form a 4-9 membered heterocycle that is unsubstituted or is substituted with one or more R¹³;
R⁴ is H;
R⁵ is selected from halogen and C₁₋₆alkyl, wherein any C₁₋₆alkyl is unsubstituted or substituted with one or more R¹³;
R⁶ is a bicyclic heteroaryl substituted with one or more R¹³;
R⁷ is selected from halogen:
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl,
each R¹¹ is independently selected from —OR¹², ═O, —C(O)C₁₋₆alkylene)CN, —C(O)C₁₋₆alkylene)OR¹⁴, —C(O)(C₁₋₆alkyl), —S(O)₂(C₁₋₆alkyl), —N(R¹⁴)C(O)C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or mom R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹²)₂, —CN, and C₁₋₆alkyl, wherein any C₁₋₆alkyl is unsubstituted or substituted with one or more R¹³;
each R²⁰ is independently selected from —OH, —OC₁₋₆alkyl, —CN, —NH₂, —NHC₁₋₆alkyl, and halogen;
R²⁰ is a 3-6 membered heterocycle including one or more heteroatoms selected from N, O, and S, wherein the heterocycle is unsubstituted or substituted with one or more R¹³;
each R²⁸ is independently selected from C₁₋₆alkyl and halogen; and
R^{a} and R^{b} are each independently selected from halogen, C₁₋₆ alkyl, —OR¹², and H, wherein any C₁₋₆alkyl is unsubstituted or is substituted with one or more R¹³.
A113. A compound represented by Formula III:

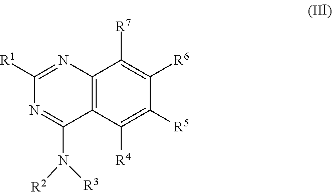

or a salt (e.g., pharmaceutically acceptable salt) thereof, wherein:
R¹ is selected from —OR⁸ and

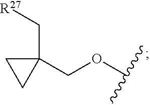

R² and R³, together with the nitrogen atom to which they are attached, form a 4-9 membered heterocycle that is unsubstituted or is substituted with one or more R¹¹, provided that when the heterocycle contains an additional nitrogen atom, the additional nitrogen atom is substituted with R¹³;
R⁴ is H;
R⁵ is selected from halogen and C₁₋₆alkyl, wherein any C₁₋₆alkyl is unsubstituted or substituted with one or more R¹³;
R⁶ is a bicyclic heteroaryl substituted with one or more R;
R⁷ is selected from halogen:
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹¹ is independently selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹²)₂, —CN, and C₁₋₆alkyl, wherein any C₁₋₆alkyl is unsubstituted or substituted with one or more R¹³;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen; and
R^{a} and R^{b} are each independently selected from halogen, C₁₋₆ alkyl, —OR¹², and H, wherein any C₁₋₆alkyl is unsubstituted or is substituted with one or more R¹³.
A114. The compound of embodiment A112 or A113, wherein R² and R³, together with the nitrogen atom to which they are attached, form a 4-6 membered heterocycle.
A115. The compound of embodiment A114, wherein R² and R³, together with the nitrogen atom to which they are attached, form a 4-6 membered heterocycle that includes 1 or 2 heteroatoms selected from O, S, and N.
A116. The compound of embodiment A115, wherein R² and R³, together with the nitrogen atom to which they are attached, form a 4-6 membered heterocycle that includes 1 or 2 heteroatoms selected from O, S, and N, wherein the heterocycle is substituted with 1-4 R¹¹.
A117. The compound of any one of embodiments A112-A116, wherein R² and R³, together with the nitrogen atom to which they are attached, form a pyrrolidine that is substituted with 0-4 R¹¹.
A118. The compound of any one of embodiments A112-A116, wherein R² and R³, together with the nitrogen atom to which they are attached, form an azetidine that is substituted with 0-4 R¹¹.
A119. The compound of any one of embodiments A112-A116, wherein R² and R³, together with the nitrogen atom to which they are attached, form a piperidine that is substituted with 0-4 R¹¹.
A120. The compound of any one of embodiments A112-A116, wherein R² and R³, together with the nitrogen atom to which they are attached, form a piperazine that is substituted with 0-4 R¹¹.
A121. The compound of any one of embodiments A112-A116, wherein R² and R³, together with the nitrogen atom to which they are attached, form a morpholine or thiomorpholine that is substituted with 0-4 R¹¹.
A122. The compound of any one of embodiments A112-A121, wherein R² and R³, together with the nitrogen atom to which they are attached, form a 4-6 membered heterocycle substituted with one or more R¹¹, wherein at least one R¹¹ is selected from —OR¹¹ and a C₁₋₆alkyl substituted with —OH.
A123. The compound of any one of embodiments A112-A121, wherein R² and R³, together with the nitrogen atom to which they are attached, form a 4-6 membered heterocycle substituted with one or more R¹¹, wherein at least one R¹¹ is an unsubstituted C₁₋₆alkyl.
A124. The compound of embodiment A112 or A113, wherein R² and R³, together with the nitrogen atom to which they are attached, form a bridged heterocycle.
A125. The compound of embodiment A124, wherein R² and R³, together with the nitrogen atom to which they are attached, form a 7-9 membered bridged heterocycle that includes 1 or 2 heteroatoms selected from O, S, and N.
A126. The compound of embodiment A125, wherein R² and R³, together with the nitrogen atom to which they are attached, form a 7-9 membered bridged heterocycle that includes 1 or 2 heteroatoms selected from O, S, and N, wherein the bridged heterocycle is substituted with 1-4 R¹¹.
A127. The compound of any one of embodiments A124-A126, wherein R² and R³, together with the nitrogen atom to which they are attached, form a bridged piperidine that is substituted with 0-4 R¹¹.
A128. The compound of any one of embodiments A124-A126, wherein R² and R³, together with the nitrogen atom to which they are attached, form a bridged piperazine that is substituted with 0-4 R¹¹.
A129. The compound of any one of embodiments A124-A126, wherein R² and R³, together with the nitrogen atom to which they are attached, form a bridged morpholine or thiomorpholine that is substituted with 0-4 R¹¹.
A130. The compound of any one of embodiments A124-A129, wherein R² and R³, together with the nitrogen atom to which they are attached, form a bridged heterocycle substituted with one or more R¹¹, wherein at least one R¹¹ is selected from —OR² and a C₁₋₆alkyl substituted with —OH.
A131. The compound of any one of embodiments A124-A130, wherein R² and R³, together with the nitrogen atom to which they are attached, form a bridged heterocycle substituted with one or more R¹¹, wherein at least one R¹¹ is an unsubstituted C₁₋₆alkyl.
A132. The compound of embodiment A112 or A113, wherein R² and R³, together with the nitrogen atom to which they are attached, form a spirocycle.
A133. The compound of embodiment A132, wherein R² and R³, together with the nitrogen atom to which they are attached, form a 4-9 membered heterocycle that is unsubstituted or is substituted with one or more R¹¹, wherein the one or more R¹¹ are independently selected from —OR¹², ═O, —C(O)C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl that is unsubstituted or substituted with one or more R²⁰.
A134. The compound of any one of embodiments A112-A133, wherein the compound is a compound according to Formula IIIA:

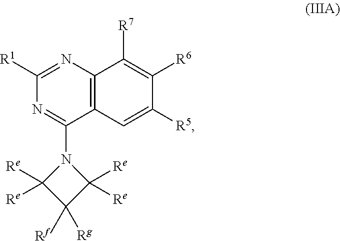

or a salt (e.g., pharmaceutically acceptable salt) thereof, wherein:
each R^{e} is independently selected from hydrogen, —OR¹², ═O, —C(O)(C₁₋₆(alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)C₁₋₆alkylene)OR¹⁴, halogen, and C₁₋₆alkyl, wherein any C₁₋₆alkyl is unsubstituted or substituted with one or more R²⁰; and
R^{f} and R^{g} are each independently selected from hydrogen, —OR¹², ═O, —C(O)(C₁₋₆₆alkylene)CN, —C(O)(C₁₋₆alkylene)OR¹², —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R⁰, or
R^{f} and R^{g} join together to form a 3-6 membered carbocycle or heterocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰.
A135. The compound of embodiment A134, wherein each R^{e}, R^{f}, and R^{g} is independently selected from hydrogen, —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰.
A136. The compound of embodiment A134, wherein each R^{e}, R^{f}, and R^{e} is independently selected from hydrogen, —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹², halogen, and C₁₋₆alkyl that is unsubstituted or substituted with one or more R²⁰;
A137. The compound of embodiment A134, wherein R^{f} and R^{g} join together to form a 3-6 membered carbocycle or heterocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R².
A138. The compound of embodiment A137, wherein R^{f} and R^{g} join together to form a 3-6 membered carbocycle or heterocycle that is unsubstituted.
A139. The compound of embodiment A137, wherein R^{f} and R^{g} join together to form a 3-6 membered carbocycle or heterocycle that is substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)C₁₋₆alkylene)OR¹⁴, halogen, and C₁₋₆alkyl, wherein any C₁₋₆alkyl is unsubstituted or substituted with one or more R²⁰.
A140. The compound of any one of embodiments A137-A139, wherein R^{f} and R^{g} join together to form a 3-6 membered carbocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹², halogen, and C₁₋₆alkyl, wherein any C₁₋₆alkyl is unsubstituted or substituted with one or more R²⁰.
A141. The compound of embodiment A140, wherein R^{f} and R^{g} join together to form a cyclopropane that is unsubstituted or substituted with one or more substituents selected from —OR¹², —O, —C(O)(C₁₋₆ alkylene)CN, —C(O(C₁₋₆alkylene)OR¹², —C(O)(C₁₋₆alkyl), —S(O)₂(C₁₋₆alkyl), —N(R¹⁴)C(O)(C₁₋₆ alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰.
A142. The compound of any one of embodiments A137-A139, wherein R^{f} and R^{g} join together to form a 3-6 membered heterocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹², halogen, and C₁₋₆alkyl, wherein any C₁₋₆alkyl is unsubstituted or substituted with one or more R²⁰.
A143. The compound of embodiment A142, wherein R^{f} and R^{g} join together to form an oxetane that is unsubstituted or substituted with one or more substituents selected from —OR¹², ═O, —C(O)(C₁₋₆alkylene)CN, —C(O(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O(C₁₋₆alkylene)OR¹⁴, halogen, and C₁₋₆alkyl, wherein any C₁₋₆alkyl is unsubstituted or substituted with one or more R¹³.
A144. The compound of any one of embodiments A112-A133, wherein the compound is a compound according to Formula IIIB:

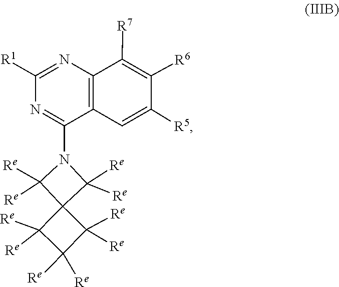

or a salt (e.g., pharmaceutically acceptable salt) thereof, wherein:
each R^{e} is independently selected from hydrogen, —OR¹², ═O, —C(O)(C₁₋₆alkylene)CN, —C(O)(C₁₋₆alkylene)OR¹⁴, —C(O(C₁₋₆alkyl), —S(O)₂(C₁₋₆alkyl), —N(R¹⁴)C(O)(C₁₋₆alkyl), —N(R¹⁴)C(O)C₁₋₆alkylene)OR¹⁴, halogen, and C₁₋₆alkyl, wherein any C₁₋₆alkyl is unsubstituted or substituted with one or more R²⁰.
A145. The compound of embodiment A144, wherein each R² is hydrogen.
A146. The compound of any one of embodiments A112-A133, wherein the compound is a compound according to Formula IIIC:

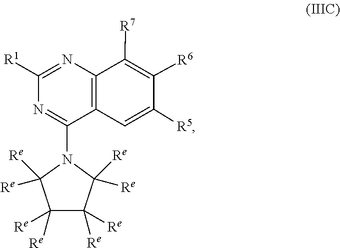

or a salt (e.g., pharmaceutically acceptable salt) thereof, wherein:
each R^{e} is independently selected from hydrogen, —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)C₁₋₆alkyl), —N(R¹⁴)C(O)C₁₋₆alkylene)OR¹², halogen, and C₁₋₆alkyl, wherein any C₁₋₆alkyl is unsubstituted or substituted with one or more R²⁰.
A147. The compound of embodiment A146, wherein each R² is hydrogen.
A148. The compound of any one of embodiments A112-A133, wherein the compound is a compound according to Formula IIID:

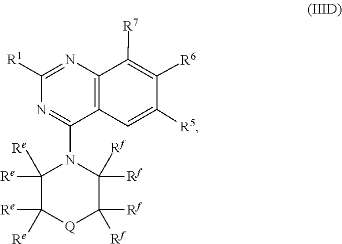

or a salt (e.g., pharmaceutically acceptable salt) thereof, wherein:
Q is selected from CR^{h}R^{j}, NR^{g}, O, S, and SO₂;
each R^{e} and R^{f} is independently selected from R¹¹ and hydrogen, wherein an R^{e} and an R^{f} can optionally join together to form a 4-6 membered ring, or a first R^{f} and a second R^{f} connected to adjacent atoms can optionally join together to form a 3-5 membered ring, or a first R^{e} and a second R^{e} connected to adjacent atoms can optionally join together to form a 3-5 membered ring;
R^{g}, when present, is R¹¹, and
R^{h} and R^{j}, when present, are independently selected from R¹¹ and hydrogen, or can optionally join together to form a 3-4 membered carbocycle or heterocycle that is unsubstituted or substituted with one or more substituents selected from —OR¹², ═O, —C(O)C₁₋₆alkylene)CN, —C(O)C₁₋₆alkylene)OR¹², —C(O)C₁₋₆alkyl), —S(O)₂(C₁₋₆alkyl), —N(R¹⁴)C(O)C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰.
A149. The compound of embodiment A148, wherein Q is O, S, or SO₂.
A150. The compound of embodiment A149, wherein Q is O.
A151. The compound of embodiment A148, wherein Q is NR^{g}.
A152. The compound of embodiment A148, wherein Q is CR^{h}R^{j}.
A153. The compound of embodiment A148, wherein the compound is a compound according to Formula IIIE:

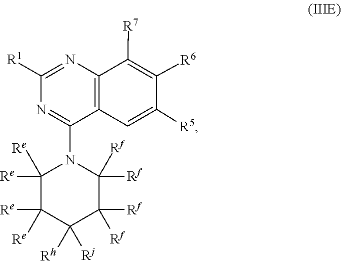

or a salt (e.g., pharmaceutically acceptable salt) thereof.
A154. The compound of embodiment A153, wherein R^{h} and R^{j} are independently selected from R¹¹ and hydrogen.
A155. The compound of embodiment A153, wherein R^{h} and Rⁱ join together to form a 3-4 membered carbocycle or heterocycle that is unsubstituted or substituted with one or more substituents selected from —OR¹², ═O, —C(O)(C₁₋₆alkylene)CN, —C(O)(C₁₋₆alkylene)OR¹², —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰.
A156. The compound of embodiment A155, wherein R^{h} and R^{j} join together to form a 3-4 membered carbocycle or heterocycle that is unsubstituted.
A157. The compound of embodiment A153, wherein R^{h} and R^{j} join together to form a 3-4 membered carbocycle or heterocycle that is substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)C₁₋₆alkyl), —N(R¹⁴)C(O)(C₁₋₆alkylene)OR¹⁶, halogen, and C₁₋₆alkyl, wherein any C₁₋₆ alkyl is unsubstituted or substituted with one or more R²⁰.
A158. The compound of embodiment A153, wherein R^{h} and R^{j} join together to form a 3-4 membered carbocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰.
A159. The compound of embodiment A158, wherein R^{h} and R^{j} join together to form a cyclobutane that is unsubstituted or substituted with one or more substituents selected from —OR¹², ═O, —C(O)(C₁₋₆alkylene)CN, —C(O(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR⁴, halogen, and C₁₋₆alkyl, wherein any C₁₋₆alkyl is unsubstituted or substituted with one or more R²⁰.
A160. The compound of embodiment A153, wherein R^{h} and R^{j} join together to form a 3-4 membered heterocycle that is unsubstituted or substituted with one or more substitutents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆ alkyl is unsubstituted or substituted with one or more R²⁰.
A161. The compound of embodiment A148, wherein the compound is a compound according to Formula IIIF, IIIG, or IIIH:

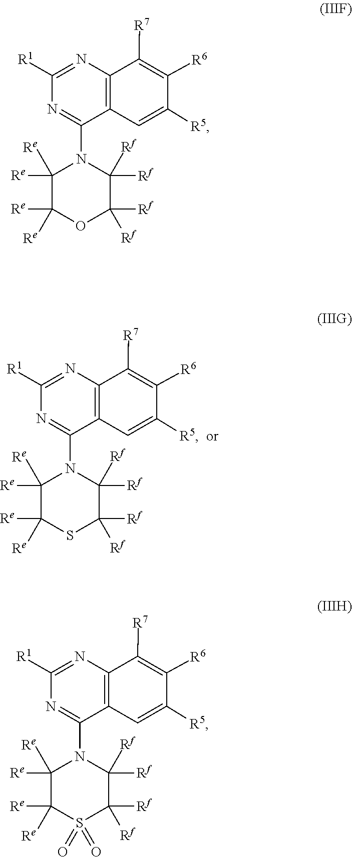

or a salt (e.g., pharmaceutically acceptable salt) thereof.
A162. The compound of embodiment A161, wherein the compound is a compound of Formula IIIF or a salt (e.g., a pharmaceutically acceptable salt) thereof.
A163. The compound of embodiment A161, wherein the compound is a compound of Formula IIIG or a salt (e.g., a pharmaceutically acceptable salt) thereof.
A164. The compound of embodiment A161, wherein the compound is a compound of Formula IIIH or a salt (e.g., a pharmaceutically acceptable salt) thereof.
A165. The compound of any one of embodiments A161-A164, wherein each R^{e} and R^{f} is independently selected from R¹¹ and hydrogen.
A166. The compound of any one of embodiments A161-A164, wherein an R^{e} and an R^{f} join together to form a 4-6 membered ring.
A167. The compound of embodiment A148, wherein the compound is a compound according to Formula IIIJ:

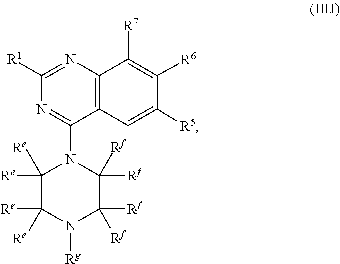

or a salt (e.g., pharmaceutically acceptable salt) thereof.
A168. The compound of embodiment A167, wherein each R^{e} and R^{f} is independently selected from R¹¹ and hydrogen.
A169. The compound of embodiment A167, wherein an R^{e} and an R^{f} join together to form a 4-6 membered ring.
A170. The compound of any one of embodiments A148-A169, wherein the compound is a compound according to Formula IIIK, Formula IIIL, Formula IIIM, Formula IIIN, Formula IIIP, Formula IIIQ, or Formula IIIR:

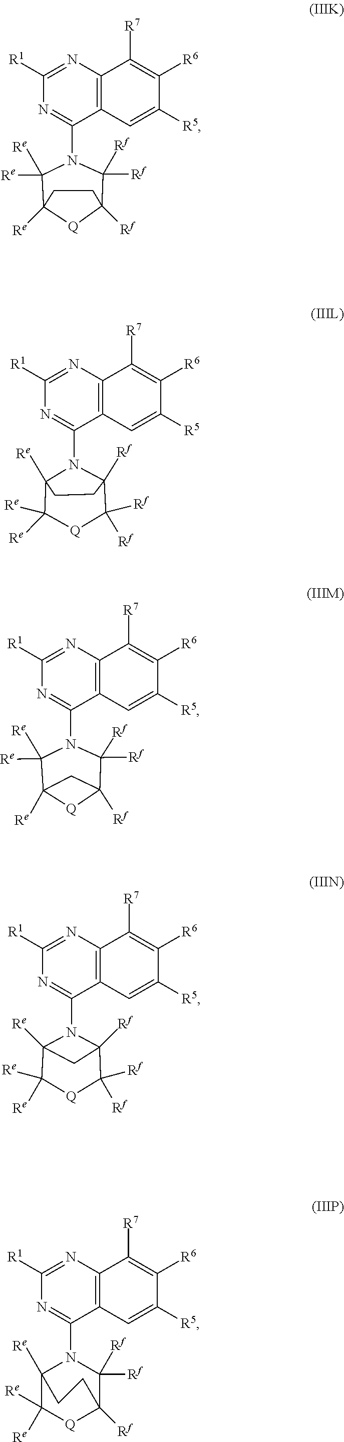

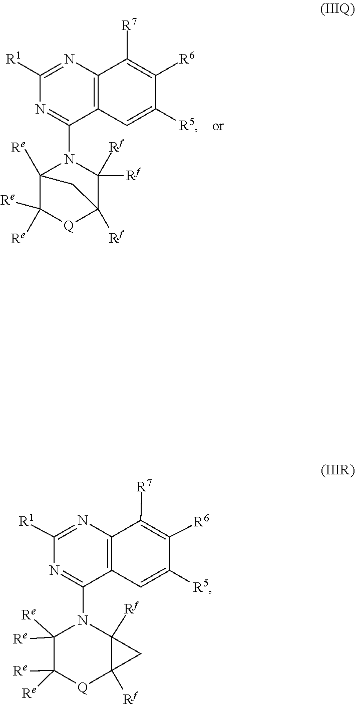

or a salt (e.g., pharmaceutically acceptable salt) wherein:
each R^{e} and R^{f} is independently selected from R¹¹ and hydrogen;
R^{g}, when present, is R¹¹, and
R^{h} and R^{j}, when present, are independently selected from R¹¹ and hydrogen.
A171. The compound of embodiment A170, wherein the compound is a compound of Formula IIIK or a salt (e.g., a pharmaceutically acceptable salt) thereof.
A172. The compound of embodiment A170, wherein the compound is a compound of Formula IIIL or a salt (e.g., a pharmaceutically acceptable salt) thereof.
A173. The compound of embodiment A170, wherein the compound is a compound of Formula IIIM or a salt (e.g., a pharmaceutically acceptable salt) thereof.
A174. The compound of embodiment A170, wherein the compound is a compound of Formula IIIN or a salt (e.g., a pharmaceutically acceptable salt) thereof.
A175. The compound of embodiment A170, wherein the compound is a compound of Formula IIIP or a salt (e.g., a pharmaceutically acceptable salt) thereof.
A176. The compound of embodiment A170, wherein the compound is a compound of Formula IIIQ or a salt (e.g., a pharmaceutically acceptable salt) thereof.
A177. The compound of embodiment A170, wherein the compound is a compound of Formula IIIR or a salt (e.g., a pharmaceutically acceptable salt) thereof.
A178. The compound of any one of embodiments A170-A177, wherein Q is selected from CR^{h}R^{j}, NR^{g}, and O.
A179. The compound of embodiment A178, wherein Q is CR^{h}R^{j}.
A180. The compound of embodiment A179, wherein R^{f} and R^{g} are independently selected from R¹¹ and hydrogen.
A181. The compound of embodiment A178, wherein Q is NR^{g}.
A182. The compound of embodiment A178, wherein Q is O.
A183. The compound of any one of embodiments A170-A177, wherein Q is S or SO₂.
A184. The compound of any one of embodiments A112-A183, wherein R¹ is selected from:

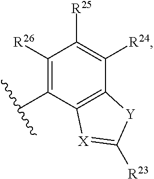

wherein:
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆alkyl, wherein any C₁₋₆alkyl is unsubstituted or substituted with one or more R¹³.
A185. The compound of any one of embodiments A112-A183, wherein R⁶ is selected from:

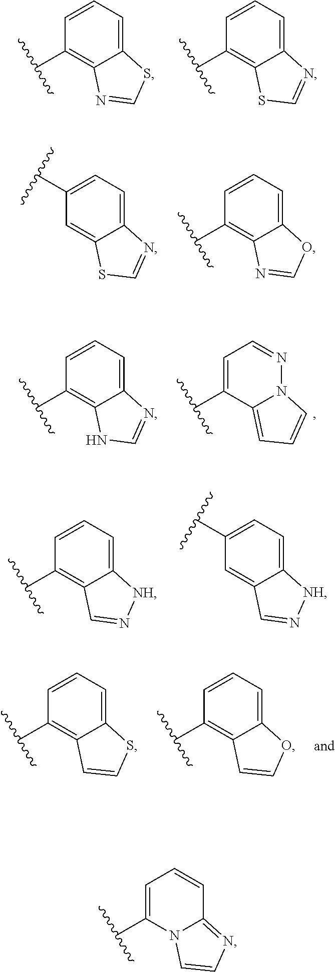

any of which is substituted with one or more R¹³.
A186. The compound of any one of embodiments A112-A183, wherein R⁶ is selected from:

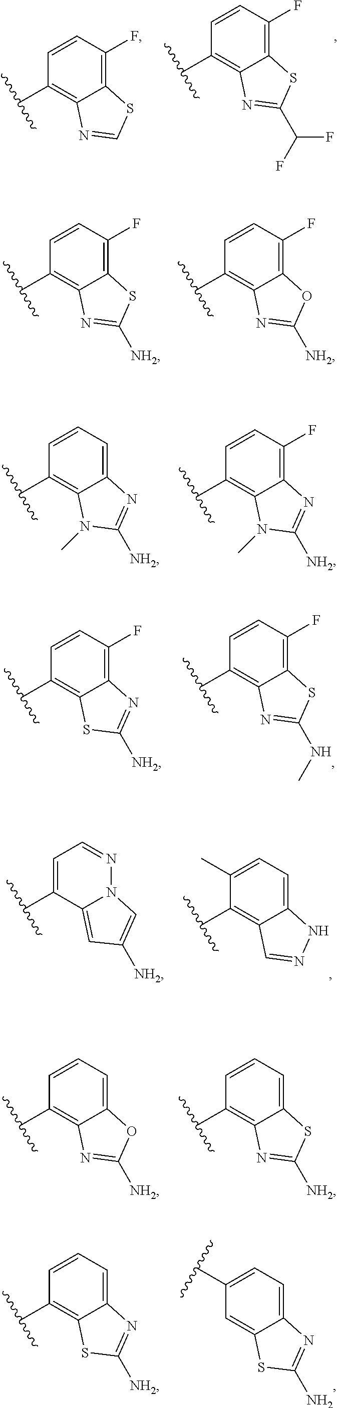

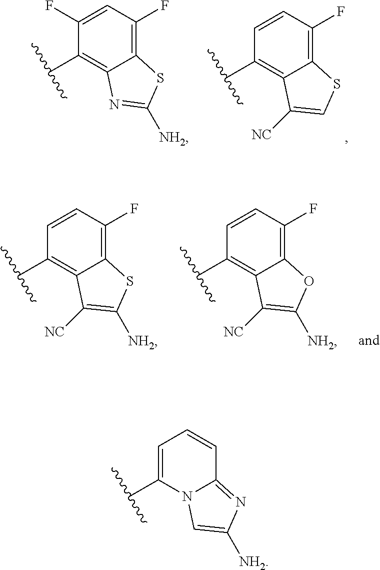 [SMILES: *c1c(F)cc(F)c2sc(N)nc12 ; *c1ccc(F)c2scc(C#N)c12 ; *c1ccc(F)c2sc(N)c(C#N)c12 ; *c1ccc(F)c2oc(N)c(C#N)c12 ; *c1cccc2nc(N)cn12]

A187. The compound of any one of embodiments A112-A183, wherein R⁶ is selected from:

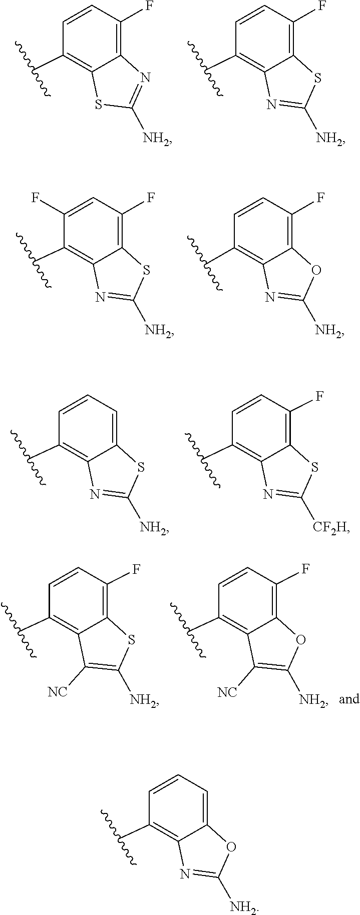

A188. The compound of any one of embodiments A112-A187, wherein R¹ is selected from —OR⁸.
A189. The compound of embodiment A188, wherein R¹ is selected from:

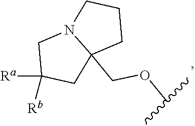

wherein R^{a} and R^{b} are each independently selected from halogen, C₁₋₆alkyl, —OR¹², and H, wherein any C₁₋₆alkyl is unsubstituted or is substituted with one or more R¹³.
A190. The compound of embodiment A189, wherein R¹ is selected from:

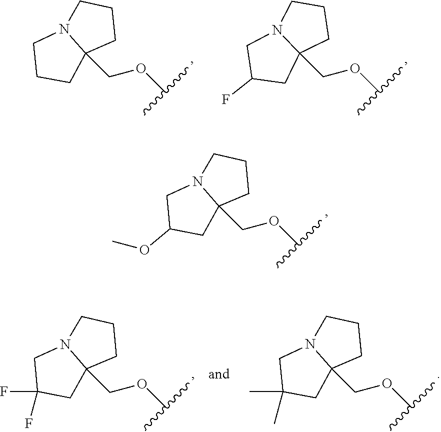 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

A191. The compound of embodiment A188, wherein R¹ is selected from:

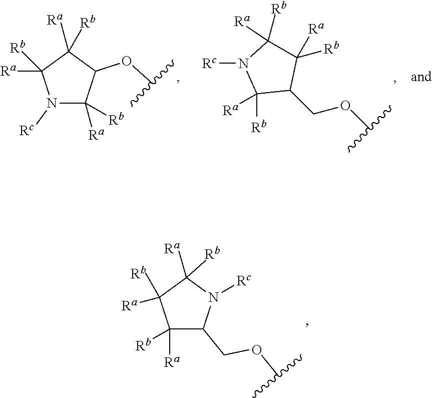

wherein each R^{a} and R^{b} is independently selected from halogen, C₁₋₆ alkyl, —OR¹², and H; and R^{c} is selected from C₁₋₆ alkyl, wherein the C₁₋₆ alkyl is unsubstituted or is substituted with one or more R¹³.
A192. The compound of embodiment A191, wherein R¹ is selected from:

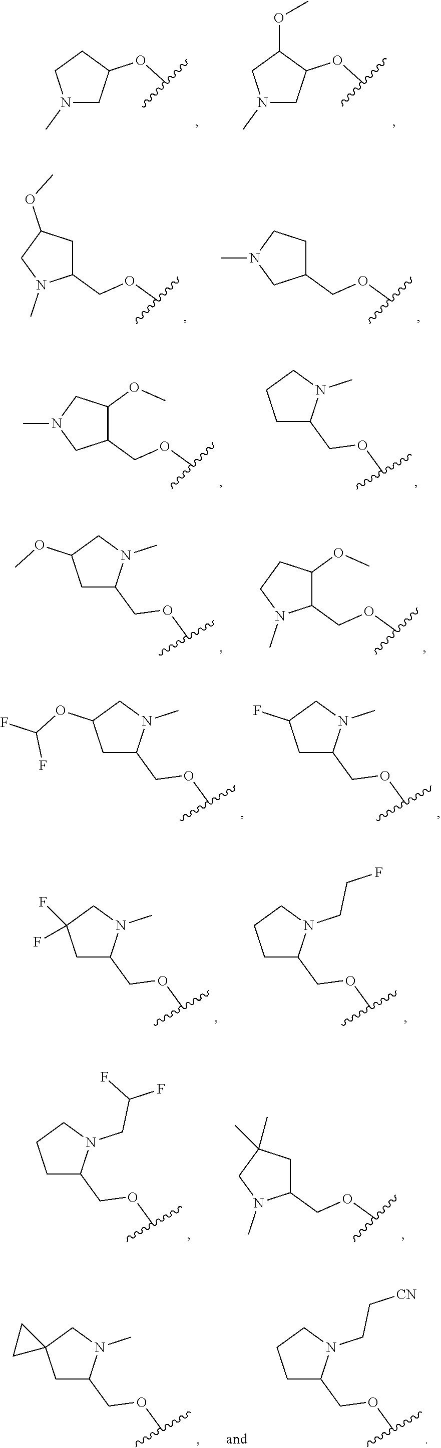

A193. The compound of any one of embodiments A112-A187, wherein R¹ is selected from

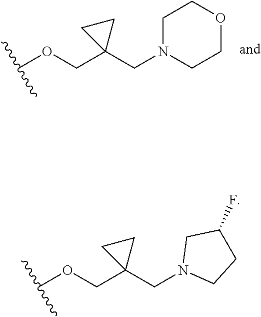 [SMILES: *OCC1(CN2CCOCC2)CC1 ; *OCC1(CN2CC[C@@H](F)C2)CC1]

A194. The compound of any one of embodiments A112-A193, wherein each R¹¹ is independently selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl that is unsubstituted or substituted with one or more R²⁰.
A195. The compound of any one of embodiments A112-A194, wherein R⁵ is a halogen (e.g., F or Cl).
A196. The compound of any one of embodiments A112-A194, wherein R⁵ is selected from C₁₋₆alkyl that is unsubstituted or substituted with one or more R¹³.
A197. The compound of embodiment A196, wherein R¹ is selected from C₁₋₆alkyl that is substituted with one or more halogens or —CN.
A198. The compound of embodiment A197, wherein R⁵ is selected from —CF₂H, —CF₃, —CH₂CN, and —CH₂CH₃.
A199. The compound of embodiment A198, wherein R⁵ is —CF₃.
A200. The compound of any one of embodiments A112-A199, wherein the compound is a not a compound included in Table 2.
A201. A compound represented by Formula IV:

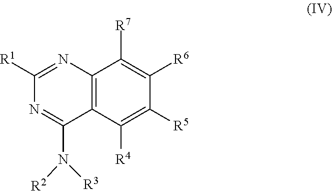

or a salt (e.g., pharmaceutically acceptable salt) thereof, wherein:
R¹ is selected from —OR³ and

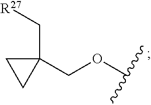

R² is selected from H and C₁₋₆alkyl, wherein any C₁₋₆ alkyl is unsubstituted or is substituted with one or more R¹³;
R³ is selected from C₁₋₆alkyl that is unsubstituted or is substituted with one or more R¹⁰;
R⁴ is H;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁶ is a bicyclic heteroaryl substituted with one or more R¹⁵;
R⁷ is selected from halogen, —CN, and H;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹⁰ is independently selected from —OR¹⁴, ═O, —CN, —N(R¹⁴)₂, a 3-6 membered carbocycle, C₁₋₆alkyl, and halogen, wherein any C₁₋₆alkyl is unsubstituted or substituted with one or more R²⁰, and wherein any 3-6 membered carbocycle is unsubstituted or substituted with one or more R¹³;
each R¹² is independently selected from C₁₋₆ alkyl, C₂₋₆ alkenyl, and H, wherein any C₁₋₆alkyl or C₁₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹²)₂, —CN, and C₁₋₆alkyl, wherein any C₁₋₆alkyl is unsubstituted or substituted with one or more R¹³;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen; and
R^{a} and R^{b} are each independently selected from halogen, C₁₋₆ alkyl, —OR¹², and H, wherein any C₁₋₆alkyl is unsubstituted or is substituted with one or more R¹³.
A202. The compound of embodiment A201, wherein the compound is a compound according to Formula IVA:

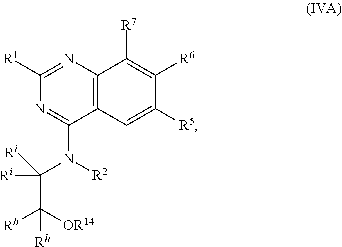

or a salt (e.g., pharmaceutically acceptable salt) thereof, wherein:
each Rⁱ and R^{h} are independently selected from H and R¹⁰.
A203. The compound of embodiment A202, wherein each R′ and R^{h} are independently selected from H and C₁₋₆alkyl, wherein any C₁₋₆alkyl is unsubstituted or substituted with one or more R¹³.
A204. The compound of embodiment A202 or A203, wherein R¹⁴ is H.
A205. The compound of any one of embodiments A201-A204, wherein R¹ is selected from:

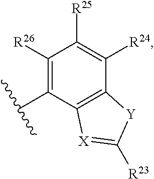

R, wherein:
   X is selected from N and C—CN;
   Y is selected from O and S;
   R² is selected from —N(R¹²)₂, C₁₋₆alkyl, and C₁₋₆alkyl-N(R¹⁴)₂, wherein any C₁₋₆alkyl is unsubstituted or substituted with one or more R¹³; and
   R²⁴, R²⁵, and R²⁶ are independently selected from H, halogen, and C₁₋₆alkyl, wherein any C₁₋₆alkyl is unsubstituted or substituted with one or more R¹³.
A206. The compound of any one of embodiments A201-A205, wherein R⁶ is selected from:

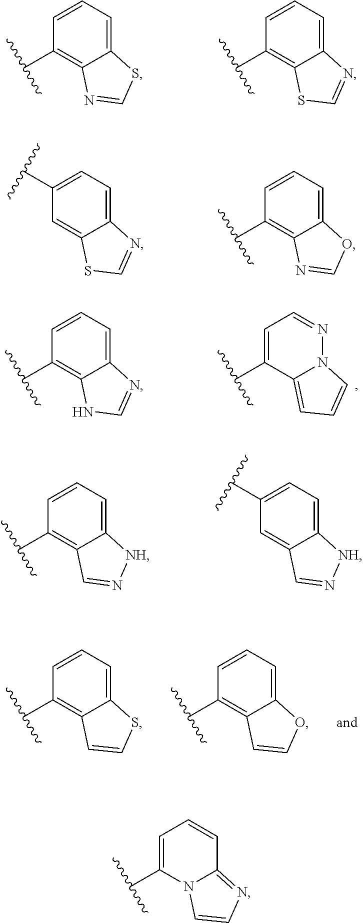

any of which is substituted with one or more R¹⁵.
A207. The compound of any one of embodiments A201-A206, wherein R⁶ is selected from:

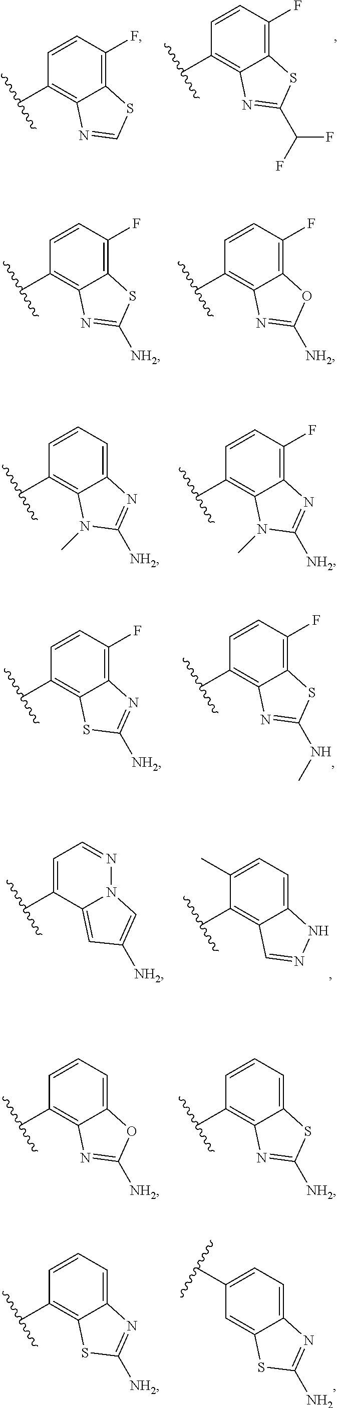

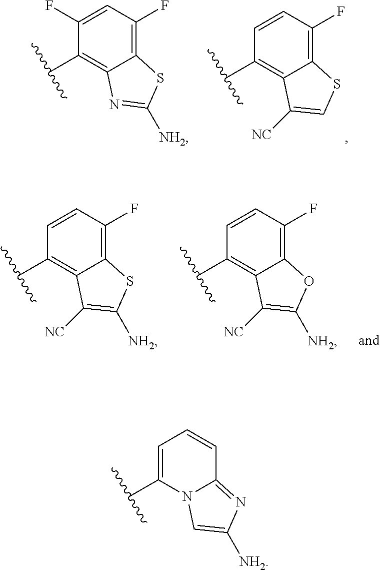 [SMILES: *c1c(F)cc(F)c2sc(N)nc12 ; *c1ccc(F)c2scc(C#N)c12 ; *c1ccc(F)c2sc(N)c(C#N)c12 ; *c1ccc(F)c2oc(N)c(C#N)c12 ; *c1cccc2nc(N)cn12]

A208. The compound of any one of embodiments A201-A207, wherein R is selected from:

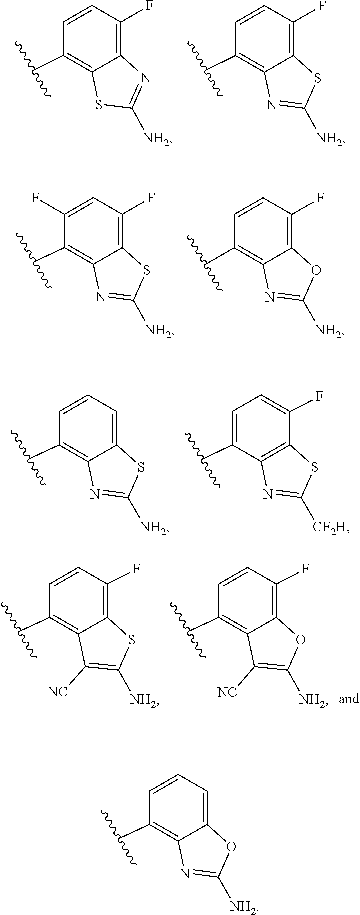

A209. The compound of any one of embodiments A201-A208, wherein the compound is a compound according to Formula IVB:

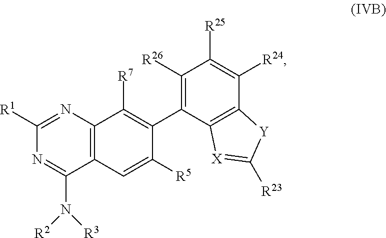

or a salt (e.g., pharmaceutically acceptable salt) thereof, wherein:
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, —OR¹², and C₁₋₆alkyl, wherein any C₁₋₆alkyl is unsubstituted or substituted with one or more R¹³.
A210. The compound of any one of embodiments A201-A209, wherein R¹ is selected from —OR⁸.
A211. The compound of embodiment A210, wherein R¹ is selected from:

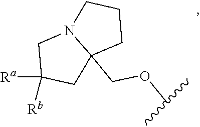

wherein R^{a} and R^{b} are each independently selected from halogen, C₁₋₆alkyl, —OR¹², and H, wherein any C₁₋₆ alkyl is unsubstituted or is substituted with one or more R¹³.
A212. Ile compound of embodiment A211, wherein R¹ is selected from:

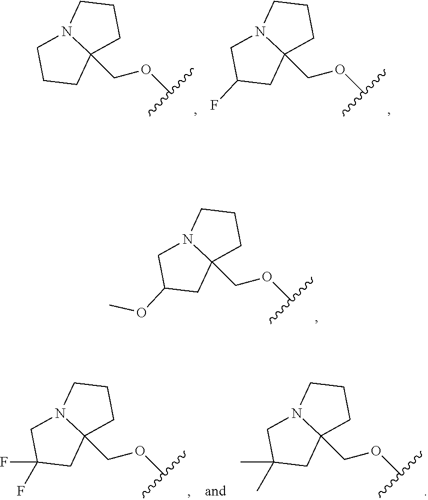

A213. The compound of embodiment A210, wherein R¹ is selected from:

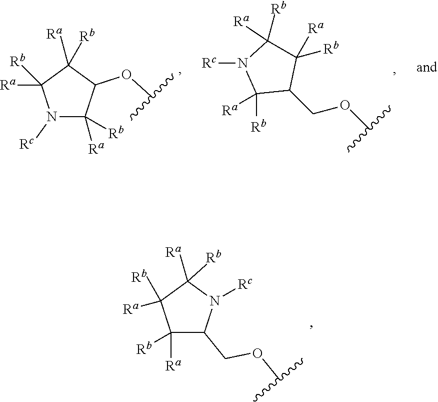

wherein each R^{a} and R^{b} is independently selected from halogen, C₁₋₆ alkyl, —OR¹², and H; and R^{e} is selected from C₁₋₆ alkyl, wherein the C₁a alkyl is unsubstituted or is substituted with one or more R¹³.
A214. The compound of embodiment A213, wherein R¹ is selected from:

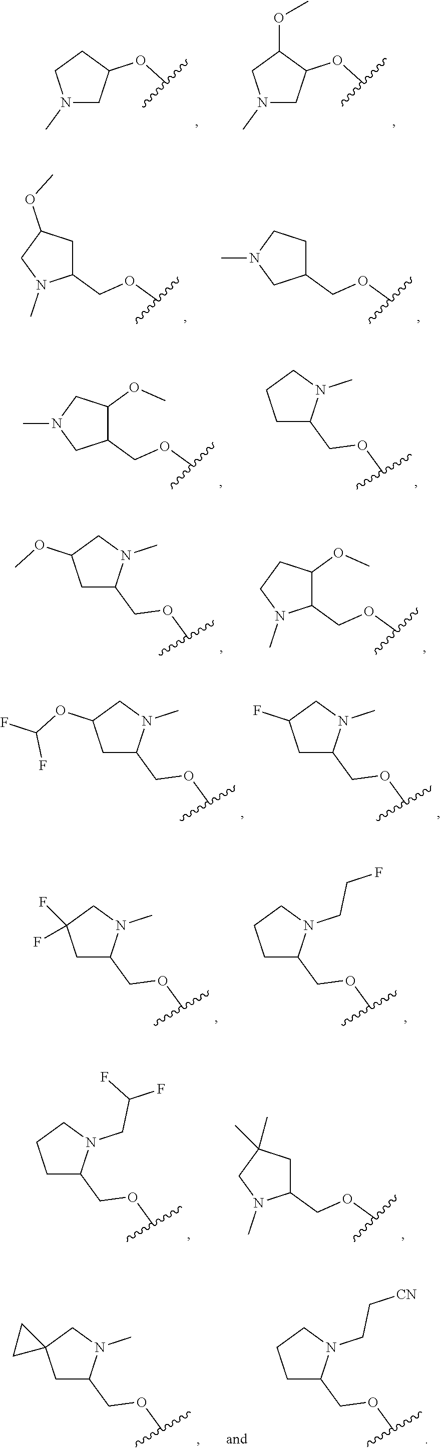

A215. The compound of any one of embodiments A201-A209, wherein R¹ is selected from

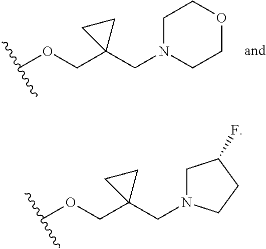 [SMILES: *OCC1(CN2CCOCC2)CC1 ; *OCC1(CN2CC[C@@H](F)C2)CC1]

A216. The compound of any one of embodiments A201-A215, wherein the compound is a compound according to Formula IVC:

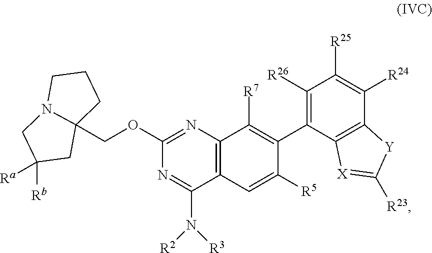

or a salt (e.g., pharmaceutically acceptable salt) thereof, wherein:
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R⁶ are independently selected from H, halogen, —OR¹², and C₁₋₆alkyl, wherein any C₁₋₆alkyl is unsubstituted or substituted with one or more R¹³.
A217. The compound of embodiment A216, wherein R is a halogen (e.g., F).
A218. The compound of embodiment A216 or A217, wherein R^{b} is H.
A219. The compound of any one of embodiments A209-A218, wherein X is C—CN, Y is S, and R³ is selected from —N(R¹²)₂.
A220. The compound of any one of embodiments A209-A219, wherein at least one of R²⁴, R²⁵, and R²⁶ is a halogen (e.g., F).
A221. The compound of any one of embodiments A201 and A209-A221, wherein R³ is selected from C₁₋₃alkyl that is unsubstituted or is substituted with one or more R¹⁰.
A222. The compound of embodiment A221, wherein R³ is selected from C₁₋₆alkyl that is substituted with one or more R¹⁰, wherein each R¹⁰ is independently selected from —OR¹⁴, ═O, —CN, —NH(R¹⁴), —N(R¹⁶)₂, a 3-6 membered carbocycle, C₁₋₆alkyl, and halogen, wherein any C₁₋₆alkyl is unsubstituted or substituted with one or more R²⁰, wherein any 3-6 membered carbocycle is unsubstituted or substituted with one or more R¹², and wherein each R¹⁶ is independently selected from C₁₋₆alkyl and C₂₋₆alkenyl.
A223. The compound of embodiment A221, wherein R³ is C₂₋₃alkyl that is unsubstituted or is substituted with one or more R¹³.
A224. The compound of embodiment A223, wherein R³ is C₂₋₃alkyl that is unsubstituted or is substituted with one or mom R¹⁰, wherein each R¹⁰ is independently selected from —OR¹⁴, ═O, —CN, —NH(R¹⁶), —N(R¹⁶)₂, a 3-6 membered carbocycle, C₁₋₆alkyl, and halogen, wherein any C₁₋₆ alkyl is unsubstituted or substituted with one or more R², wherein any 3-6 membered carbocycle is unsubstituted or substituted with one or more R², and wherein each R⁶ is independently selected from C₁₋₆akyl and C₂₋₆alkenyl.
A225. The compound of embodiment A224, wherein R³ is C₂₋₃alkyl that is unsubstituted.
A226. The compound of embodiment A224, wherein R³ is C₂₋₃alkyl that is substituted with one or more R¹⁰, wherein the one or more R¹⁰ are selected from —OR¹⁴, —CN, C₁₋₆alkyl, and halogen, wherein any C₁₋₆alkyl is unsubstituted or substituted with one or more R²⁰.
A227. The compound of embodiment A226, wherein R³ is C₂₋₃-alkyl that is substituted with one or more R¹⁰, wherein the one or more R¹⁰ are selected from —OR¹⁴ and C₁₋₆alkyl, wherein any C₁₋₆alkyl is unsubstituted or substituted with one or more R²⁰.
A228. The compound of any one of embodiments A223, A226, and A227, wherein R³ is C₂₋₃alkyl that is substituted with one or more R¹⁰, wherein the one or more R¹⁰ are halogen.
A229. The compound of embodiment A228, wherein R³ is C₂₋₃alkyl that is substituted with one or more F.
A230. The compound of any one of embodiments A201 and A209-A221, wherein the moiety

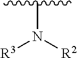

is selected from:

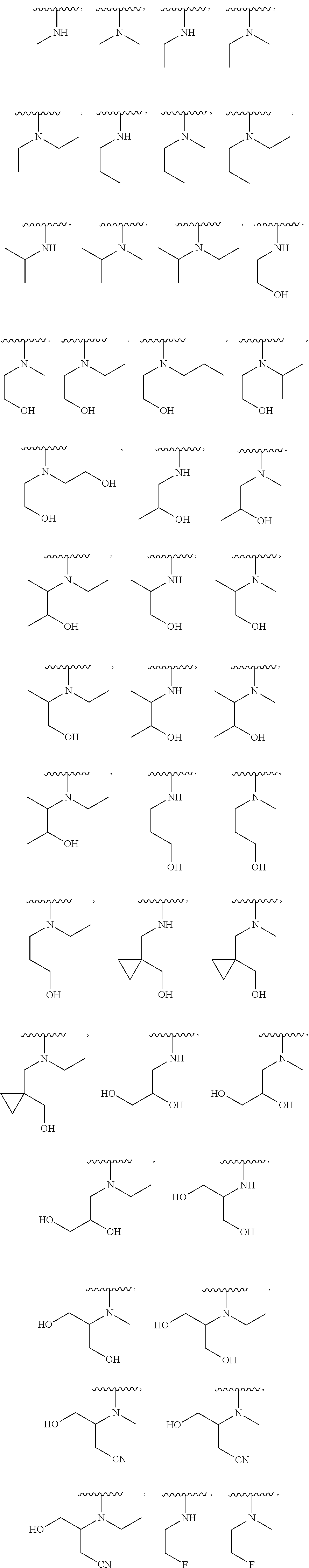

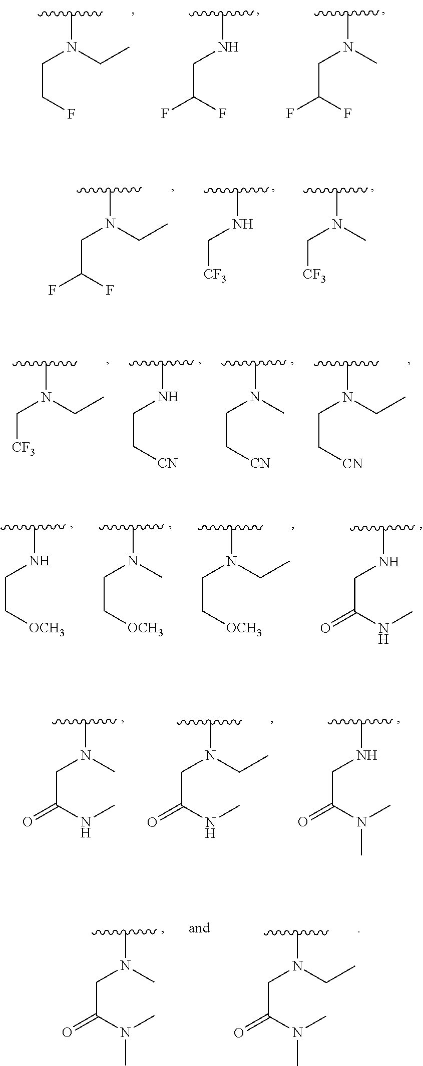

A231. The compound of any one of embodiments A201-A230, wherein R² is selected from H and C₁₋₆alkyl that is unsubstituted.
A232. The compound of embodiment A231, wherein R² is H
A233. The compound of any one of embodiments A201-A230, wherein R² is selected from C₁₋₆alkyl that is unsubstituted or is substituted with one or more R¹³
A234. The compound of embodiment A233, wherein R² is selected from C₁₋₂alkyl that is unsubstituted.
A235. The compound of any one of embodiments A201-A234, wherein R⁵ is H.
A236. The compound of any one of embodiments A201-A234, wherein R⁵ is a halogen (e.g., F or Cl).
A237. The compound of any one of embodiments A201-A234, wherein R³ is selected from C₁₋₆alkyl that is unsubstituted or substituted with one or more R¹³.
A238. The compound of embodiment A237, wherein R¹ is selected from C₁₋₆alkyl that is substituted with one or more halogens or —CN.
A239. The compound of embodiment A238, wherein R¹ is selected from —CF₂H, —CF₃, —CH₂CN, and —CH₂CH₃.
A240. The compound of embodiment A239, wherein R¹ is —CF₃.
A241. The compound of any one of embodiments A201-A240, wherein R¹ is H.
A242. The compound of any one of embodiments A201-A240, wherein R¹ is a halogen (e.g., F or Cl).
A243. The compound of any one of embodiments A201-A240, wherein R¹ is —CN.
A244. The compound of any one of embodiments A201-A243, wherein the compound is a not a compound included in Table 3.
A245. A compound shown in Table 5, or a salt (e.g., pharmaceutically acceptable salt) thereof.
A246. A pharmaceutical composition comprising a compound of any one of embodiments A I-A245, or a salt (e.g., pharmaceutically acceptable salt) thereof, and a pharmaceutically acceptable excipient.
A247. A compound of any one of embodiments A1-A245, or a salt (e.g., pharmaceutically acceptable salt) thereof, for use as a medicament.
A248. The compound of embodiment A247, wherein the medicament is useful in the prevention or treatment of a disease, disorder, or condition ameliorated by the inhibition of KRAS having a Q61H, G13D, G12D, G12V, G12C, G12S, G12A, or G12R mutation, or wild-type KRAS.
A249. The compound of embodiment A248, wherein the medicament is useful in the prevention or treatment of a disease, disorder, or condition ameliorated by the inhibition of wild-type KRAS.
A250. The compound of embodiment A248, wherein the medicament is useful in the prevention or treatment of a disease, disorder, or condition ameliorated by the inhibition of KRAS having a G12D, G12R, or G12V mutation.
A251. The compound of any one of embodiments A247-A250, wherein the medicament is useful in the prevention or treatment of a cancer.
A252. The compound of embodiment A251, wherein the cancer is selected from the group consisting of pancreatic cancer, colorectal cancer, and lung cancer.
A253. A compound of any one of embodiments A1-A245, or a salt (e.g., pharmaceutically acceptable salt) thereof, for use in the treatment of a disease, disorder, or condition.
A254. The compound of embodiment A253, wherein the disease, disorder, or condition is a cancer.
A255. The compound of embodiment A254, wherein the cancer is selected from the group consisting of pancreatic cancer, colorectal cancer, and lung cancer.
A256. The compound of any one of embodiments A245-A255, wherein the compound is used in the treatment of a disease, disorder, or condition in a subject in need thereof.
A257. A compound of any one of embodiments A1-A245, or a salt (e.g., pharmaceutically acceptable salt) thereof, for use in the manufacture of a medicament.
A258. The compound of embodiment A257, wherein the medicament is useful in the prevention or treatment of a disease, disorder, or condition ameliorated by the inhibition of KRAS having a Q61H, G13D, G12D, G12V, G12C, G12S, G12A, or G12R mutation, or wild-type KRAS.
A259 The compound of embodiment A258, wherein the medicament is useful in the prevention or treatment of a disease, disorder, or condition ameliorated by the inhibition of wild-type KRAS.
A260 The compound of embodiment A258, wherein the medicament is useful in the prevention or treatment of a disease, disorder, or condition ameliorated by the inhibition of KRAS having a G12D, G12R, or G12V mutation.
A261. The compound of any one of embodiments A257-A260, wherein the medicament is useful in the treatment of a cancer.
A262. The compound of embodiment A261, wherein the cancer is selected from the group consisting of pancreatic cancer, colorectal cancer, and lung cancer.
A263. A method, comprising administering a therapeutically effective amount of a compound of any one of embodiments A1-A245, or a salt (e.g., pharmaceutically acceptable salt) thereof, to a subject in need thereof.
A264. The method of embodiment A263, wherein the subject has a disease, disorder, or condition ameliorated by the inhibition of KRAS having a Q61H, G13D, G12D, G12V, G12C, G12S, G12A, or G12R mutation, or wild-type KRAS.
A265. The method of embodiment A264, wherein the disease, disorder, or condition is ameliorated by the inhibition of wild-type KRAS.
A266. The compound of embodiment A264, wherein the disease, disorder, or condition is ameliorated by the inhibition of KRAS having a G12D, G12R, or G12V mutation.
A267. The method of any one of embodiments A263-A266, wherein the subject has a cancer.
A268. The method of embodiment A267, wherein the subject was previously diagnosed with the cancer.
A269. The method of embodiment A267, wherein the subject has previously undergone a treatment regimen for the cancer.
A270. The method of embodiment A268, wherein the subject has previously entered remission from the cancer.
A271. The method of any one of embodiments A259-A270, wherein the cancer is selected from the group consisting of pancreatic cancer, colorectal cancer, and lung cancer.
A272. The method of any one of embodiments A263-A271, wherein the compound, or the salt thereof, is administered in combination with an additional therapeutic agent.
A273. The use of a compound of any one of embodiments A1-A245, or a salt (e.g., pharmaceutically acceptable salt) thereof for the manufacture of a medicament for the treatment of a cancer.
A274. The use of embodiment A273, wherein the cancer is selected from the group consisting of pancreatic cancer, colorectal cancer, and lung cancer.
A275. A method, comprising contacting a KRAS protein with a compound of any one of embodiments A1-A245, or a salt (e.g., pharmaceutically acceptable salt) thereof.
A276. The method of embodiment A277, wherein contacting the KRAS protein with the compound modulates KRAS.
A277. The method of embodiment A275 or A276, wherein the KRAS protein has a Q61H, G13D, G12D, G12V, G12C, G12S, G12A, or G12R mutation.
A278. The method of embodiment A275 or A276, wherein the KRAS protein is a wild-type KRAS protein.
A279. The method of any one of embodiments A275-A278, wherein the KRAS protein is in an active (GTP-bound) state.
A280. The method of any one of embodiments A275-A278, wherein the KRAS protein is in an inactive (GDP-bound) state.
A281. The method of any one of embodiments A275-A280, wherein the KRAS protein is located within a cell.
A282. The method of embodiment A281, wherein the cell is located within a subject.
A283. The method of embodiment A282, wherein the subject is a human.
A284. The method of embodiment A282 or A283, wherein the subject has a cancer.
A285. The method of embodiment A284, wherein the cancer is selected from the group consisting of pancreatic cancer, colorectal cancer, and lung cancer.
A286. A method of inhibiting the function of a wild-type KRAS protein or a KRAS protein having a Q61H, G13D, G12D, G12V, G12C, G12S, G12A, or G12R mutation, comprising contacting the KRAS protein with a compound of any one of embodiments A1-A245, or a salt (e.g., pharmaceutically acceptable salt) thereof.
A287. The method of embodiment A286, wherein the KRAS protein is a wild-type KRAS protein.
A288. The method of embodiment A286, wherein the KRAS protein has a Q61H, G13D, G12D, G12V, G12C, G12S, G12A, or G12R mutation.
A289. The method of embodiment A288, wherein the KRAS protein has a G12D, G12V, or G12R mutation.
A290. The method of any one of embodiments A286-A289, wherein the KRAS protein is in an active (GTP-bound) state.
A291. The method of any one of embodiments A286-A289, wherein the KRAS protein is in an inactive (GDP-bound) state.
A292. The method of any one of embodiments A286-A291, wherein the KRAS protein is located within a cell.
A293. The method of embodiment A292, wherein the cell is located within a subject.
A294. The method of embodiment A293, wherein the subject is a human.
A295. The method of embodiment A293 or A294, wherein the subject has a cancer.
A296. The method of embodiment A295, wherein the cancer is selected from the group consisting of pancreatic cancer, colorectal cancer, and lung cancer.
A297. A compound capable of inhibiting a wild-type KRAS protein or a KRAS protein having a Q61H, G13D, G12D, G12V, G12C, G12S, G12A, or G12R mutation in both its active (GTP-bound) and inactive (GDP-bound) state.
A298. The compound of embodiment A297, wherein the compound:
   (i) has IC₅₀≤0.1 μM, 0.1 μM<IC₅₀≤1 μM, 1 μM<IC₅₀≤10 μM, or 10 μM<IC₅₀ in the assay of Biological Example 1 (e.g., a protein:protein interaction (PPI) Homogenous Time Resolved Fluorescence (HTRF) analysis of 50 nM Avi-KRAS G12D (amino acids 1-169) GppNHp/RAF1 RBD-3×FLAG (52-151); 50 nM Avi-KRAS G12R (amino acids 1-169) GppNHp/RAF1 RBD-3×FLAG (52-151); 50 nM Avi-KRAS G12V (amino acids 1-169) GppNHp/RAF1 RBD-3×FLAG (52-151); 50 nM Avi-KRAS WT (amino acids 1-169) GppNHp/RAF1 RBD-3×FLAG (52-151); and/or 75 nM Avi-RAF1 RBD-3×FLAG; and/or
   (ii) has IC₅₀≤0.1 μM, 0.1 μM<IC₅₀<1 μM, or IC₅₀>1 μM in the assay of Biological Example 2 (e.g., cell-based pERK HTRF assay in GP2d (G12D) and SW620 (G12V) cell).
A299. The compound of embodiment A298, wherein the compound:
   (i) has IC₅₀≤0.1 μM or 0.1 μM<IC₅₀≤1 μM in the assay of Biological Example 1 (e.g., a protein:protein interaction (PPI) Homogenous Time Resolved Fluorescence (HTRF) analysis of 50 nM Avi-KRAS G12D (amino acids 1-169) GppNIIp/RAF1 RBD-3×FLAG (52-151): 50 nM Avi-KRAS G12R (amino acids 1-169) GppNHp/RAF1 RBD-3×FLAG (52-151); 50 nM Avi-KRAS G12V (amino acids 1-169) GppNHp/RAF1 RBD-3×FLAG (52-151); 50 nM Avi-KRAS WT (amino acids 1-169) GppNHp/RAF1 RBD-3×FLAG (52-151); and/or 75 nM Avi-RAF1 RBD-3×FLAG; and/or
   (ii) has IC₅₀≤0.1 μM or 0.1 μM<IC₅₀≤1 μM in the assay of Biological Example 2 (e.g., cell-based pERK HTRF assay in GP2d (G12D) and SW620 (G12V) cell).
A300. The compound of any one of embodiments A297-A299, wherein the compound is capable of irreversibly binding the KRAS protein.
A301. The compound of any one of embodiments A297-A299, wherein the compound is capable of reversibly binding the KRAS protein.
A302. The compound of any one of embodiments A297-A301, wherein the compound is a compound according to any one of embodiments A1-A245.

The following numbered embodiments, while non-limiting, are exemplary of certain aspects of the present disclosure:
B1. A compound represented by Formula II′:

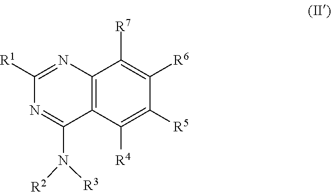

or a salt (e.g., pharmaceutically acceptable salt) thereof, wherein:
R¹ is selected from —OR^{g} and

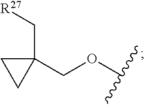

R² is selected from H and C₁₋₆alkyl, wherein any C₁₋₆ alkyl is unsubstituted or is substituted with one or more R¹³;
R³ is selected from a 4-6 membered heterocycle that is unsubstituted or substituted with one or more R¹⁰;
R⁴ is H;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹⁰ is independently selected from —OR¹², ═O, —C(O)(C₁₋₆alkylene)CN, —C(O)(C₁₋₆alkylene)OH, —C(O)(C₁₋₆alkyl), —C(O)N(R¹⁴)₂, —C(O)OR¹², —C(O)(3-6 membered carbocycle), —S(O)₂(C₁₋₆alkyl), halogen, and C₁₋₆alkyl, wherein any C₁₋₆alkyl is unsubstituted or substituted with one or more R²⁰, wherein any 3-6 membered carbocycle is unsubstituted or substituted with one or more R¹² or R², and wherein two R¹⁰s optionally join together to form, together with the atom(s) to which they are attached, a 3-6 membered carbocycle or heterocycle;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁰)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹³;
each R²⁰ is independently selected from —OH, —OC₁₋₆alkyl, ═O, —CN, —NH₂, —NHC₁₋₆alkyl, a 3-6 membered carbocycle, and halogen:
R²⁷ is a 3-6 membered heterocycle including one or more heteroatoms selected from N, O, and S, wherein the heterocycle is unsubstituted or substituted with one or more R²¹;
each R²⁸ is independently selected from C₁₋₆alkyl and halogen; and
R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.
B2. A compound represented by Formula II:

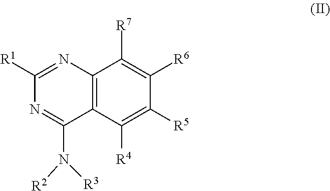

or a salt (e.g., pharmaceutically acceptable salt) thereof, wherein:
R¹ is selected from —OR⁸ and

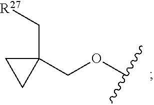

R² is selected from H and C₁₋₆alkyl, wherein any C₁₋₆ alkyl is unsubstituted or is substituted with one or more R¹³;
R³ is selected from a 4-6 membered heterocycle that is unsubstituted or substituted with one or more R¹⁰, provided that when the heterocycle contains a nitrogen atom, the nitrogen atom is substituted with R¹⁰;
R⁴ is H;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen:
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹⁰ is independently selected from —OR¹², ═O, —C(O)(C₁₋₆alkylene)CN, —C(O(C₁₋₆alkylene)OH, —C(O)(C₁₋₆alkyl), —C(O)N(R¹⁴); —C(O)OR¹⁴, —C(O)(3-6 membered carbocycle), —S(O)₂(C₁₋₆alkyl), halogen, and C₁₋₆alkyl, wherein any C₁₋₆alkyl is unsubstituted or substituted with one or more R²⁰, wherein any 3-6 membered carbocycle is unsubstituted or substituted with one or more R¹² or R²⁰, and wherein two R¹⁰s optionally join together to form, together with the atom(s) to which they are attached, a 3-6 membered carbocycle or heterocycle;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹³;
each R²⁰ is independently selected from —OH, —OC₁₋₆alkyl, ═O, —CN, —NH₂, —NHC₁₋₆alkyl, a 3-6 membered carbocycle, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen; and
R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.
B3. The compound of embodiment B1 or B2, wherein R³ is a 4-6 membered heterocycle that includes 1 heteroatom selected from O, S, and N.
B4. The compound of embodiment B3, wherein R³ is a 4-6 membered heterocycle that includes 1 heteroatom selected from O, S, and N, wherein the heterocycle is substituted with 1-4 R¹⁰, provided that when the heterocycle contains a nitrogen atom, the nitrogen atom is substituted with R¹⁰.
B5. The compound of any one of embodiments B1-B4, wherein R³ is a pyrrolidine that is substituted with 0-4 R¹⁰, provided that the nitrogen atom is substituted with R¹⁰.
B6. The compound of any one of embodiments B1-B4, wherein R³ is an oxetane or thietane that is substituted with 0-4 R¹⁰.
B7. The compound of any one of embodiments B1-B4, wherein R³ is a tetrahydrofuran or tetrahydrothiophene that is substituted with 0-4 R¹⁰.
B8. The compound of any one of embodiments B1-B4, wherein R³ is a 4-6 membered heterocycle that is substituted with one or more R¹⁰, wherein at least one R¹⁰ is selected from —OR¹² and a C₁₋₆ alkyl substituted with —OH.
B9. The compound of any one of embodiments B1-B8, wherein R³ is a 4-6 membered heterocycle that is substituted with one or more R¹⁰, wherein at least one R¹⁰ is an unsubstituted C₁₋₆alkyl.
B10. The compound of any one of embodiments B1-B9, wherein R³ is selected from:

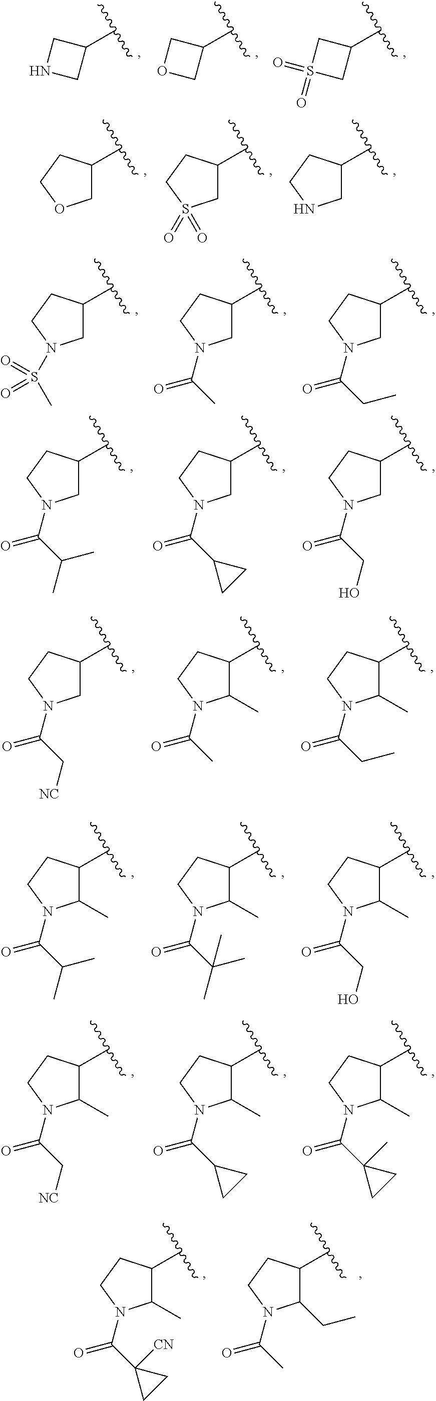

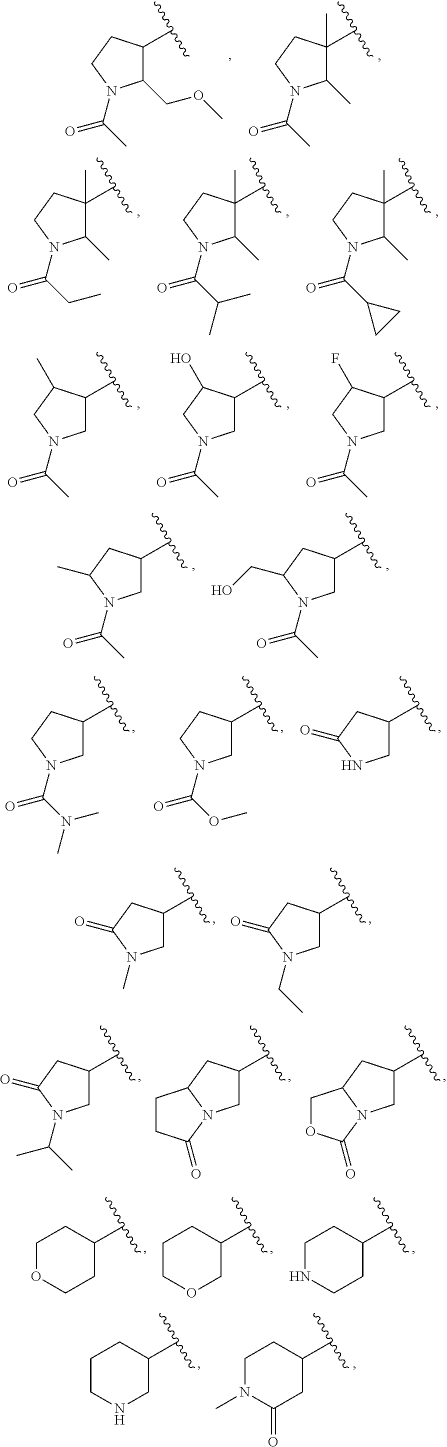

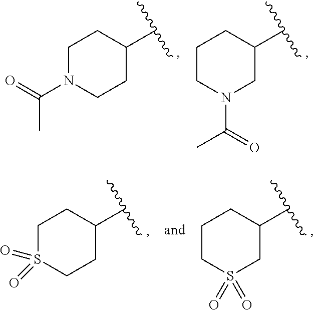

any of which is optionally further substituted with one or more R¹⁰.
B11. The compound of any one of embodiments B1-B10, wherein the compound is a compound according to Formula IIA, IIB, IIC, IID, IE, IIF, IIG, IIH, IIJ, IIIK, IIL, IIM, IN, IIIP, IIQ, IIR, IIS, IIT, IIU, IIV, IIW, IIX, IIY, or IIZ:

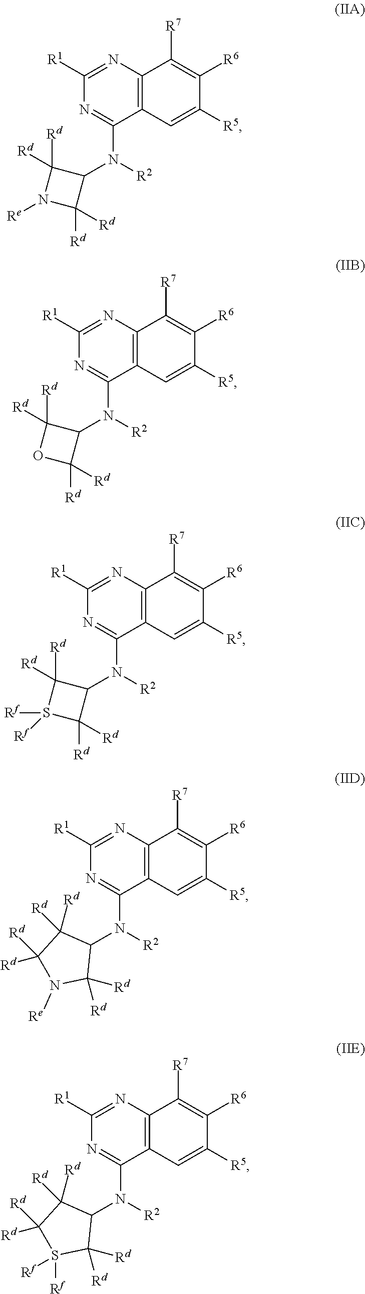

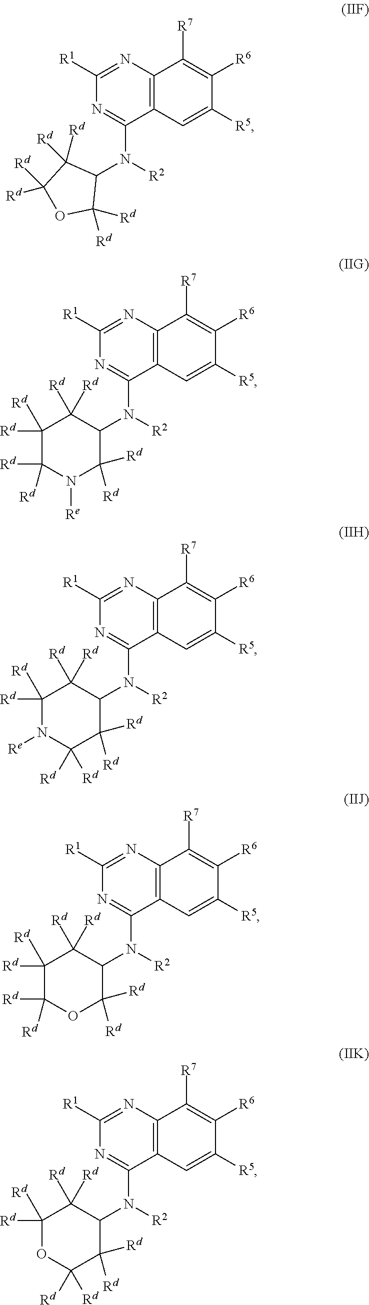

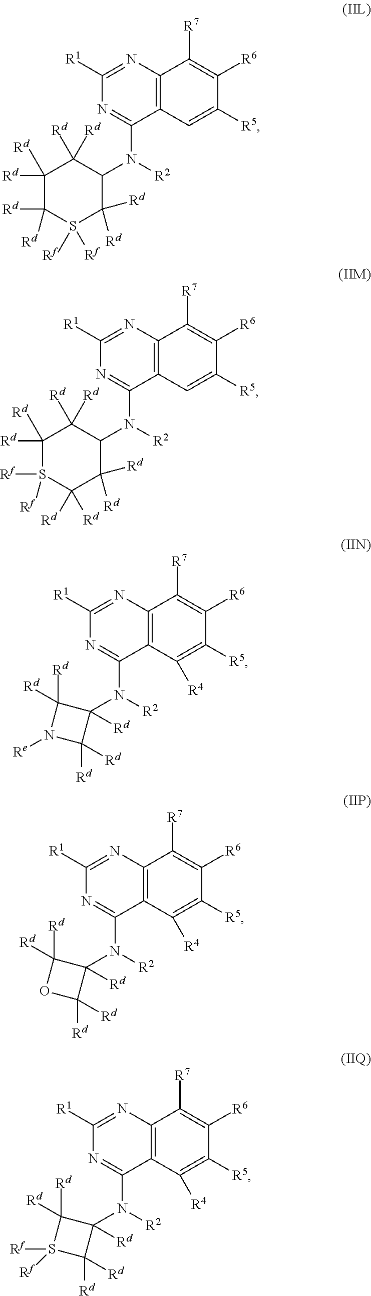

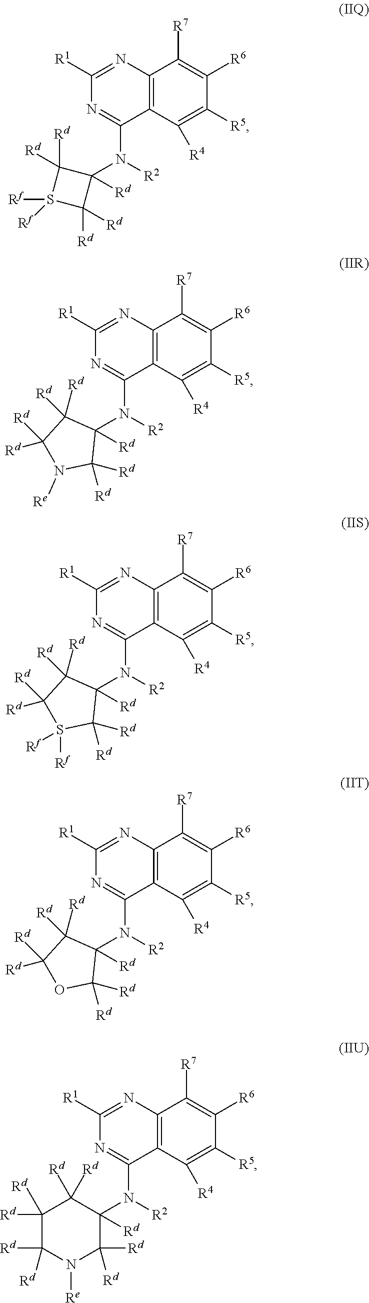

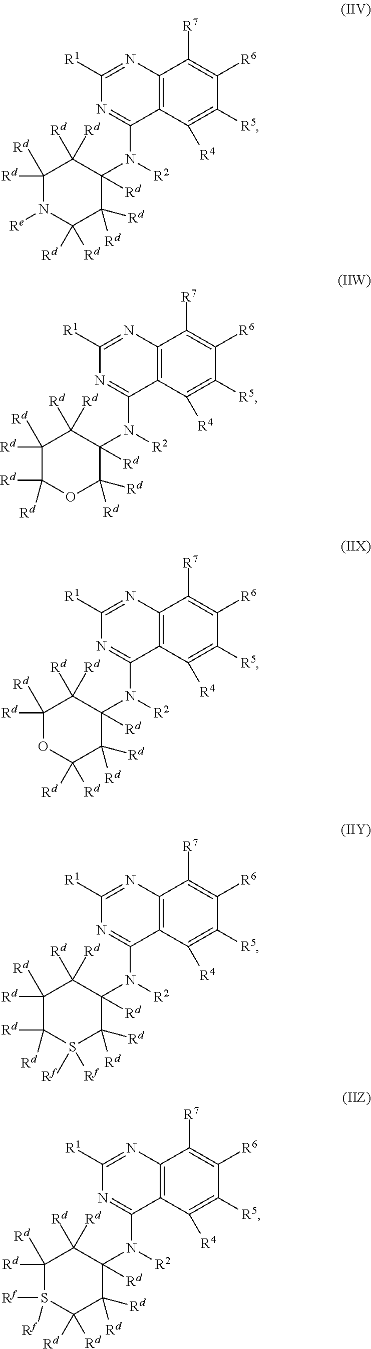

or a salt (e.g., pharmaceutically acceptable salt) thereof, wherein:
each R^{d} is independently selected from H, —OR¹², ═O, —C(O)(C₁₋₆alkylene)CN, —C(O)C₁₋₆alkylene)OH, —C(O)(C₁₋₆alkyl), —C(O)N(R¹⁴)₂, —C(O)(3-6 membered carbocycle), —S(O)₂(C₁₋₆alkyl), halogen, and C₁₋₆alkyl, wherein any C₁₋₆alkyl is unsubstituted or substituted with one or more R²⁰;
R^{e}, if present, is selected from —C(O)(C₁₋₆alkyl)CN, —C(O(C₁₋₆alkyl)OH, —C(O)C₁₋₆alkyl). —C(O)N(R¹⁴)₂, —C(O)OR¹², —C(O)(3-6 membered carbocycle), —S(O)₂(C₁₋₆alkyl), and C₁₋₆alkyl, wherein any C₁₋₆alkyl is unsubstituted or substituted with one or more R²⁰, and wherein any 3-6 membered carbocycle is unsubstituted or substituted with one or more R¹² or R²⁰; and
each R^{f} is optionally absent and, if present, is selected from ═O, —NH₂, —NHC₁₋₆alkyl, and —N(C₁₋₆alkyl)₂.
B12. The compound of embodiment B11, wherein the compound is a compound according to Formula HA, or a salt (e.g., pharmaceutically acceptable salt) thereof.
B13. The compound of embodiment B1, wherein the compound is a compound according to Formula IIB, or a salt (e.g., pharmaceutically acceptable salt) thereof.
B14. The compound of embodiment B1, wherein the compound is a compound according to Formula HC, or a salt (e.g., pharmaceutically acceptable salt) thereof.
B15. The compound of embodiment B1, wherein the compound is a compound according to Formula HD, or a salt (e.g., pharmaceutically acceptable salt) thereof.
B16. The compound of embodiment B11, wherein the compound is a compound according to Formula IIE, or a salt (e.g., pharmaceutically acceptable salt) thereof.
B17 The compound of embodiment B11, wherein the compound is a compound according to Formula IIF, or a salt (e.g., pharmaceutically acceptable salt) thereof.
B18. The compound of embodiment B11, wherein the compound is a compound according to Formula IIG, or a salt (e.g., pharmaceutically acceptable salt) thereof.
B19. The compound of embodiment B11, wherein the compound is a compound according to Formula IIH or a salt (e.g., pharmaceutically acceptable salt) thereof.
B20. The compound of embodiment B11, wherein the compound is a compound according to Formula IIJ, or a salt (e.g., pharmaceutically acceptable salt) thereof.
B21. The compound of embodiment B11, wherein the compound is a compound according to Formula IIK, or a salt (e.g., pharmaceutically acceptable salt) thereof.
B22. The compound of embodiment B11, wherein the compound is a compound according to Formula IIL, or a salt (e.g., pharmaceutically acceptable salt) thereof.
B23. The compound of embodiment B11, wherein the compound is a compound according to Formula IIM, or a salt (e.g., pharmaceutically acceptable salt) thereof.
B24. The compound of embodiment B11, wherein the compound is a compound according to Formula IIN, or a salt (e.g., pharmaceutically acceptable salt) thereof.
B25. The compound of embodiment B11, wherein the compound is a compound according to Formula IIP, or a salt (e.g., pharmaceutically acceptable salt) thereof.
B26. The compound of embodiment B11, wherein the compound is a compound according to Formula IIQ, or a salt (e.g., pharmaceutically acceptable salt) thereof.
B27. The compound of embodiment B11, wherein the compound is a compound according to Formula IIR, or a salt (e.g., pharmaceutically acceptable salt) thereof.
B28. The compound of embodiment B11, wherein the compound is a compound according to Formula IIS, or a salt (e.g., pharmaceutically acceptable salt) thereof.
B29. The compound of embodiment B11, wherein the compound is a compound according to Formula IIT, or a salt (e.g., pharmaceutically acceptable salt) thereof.
B30. The compound of embodiment B11, wherein the compound is a compound according to Formula IIU, or a salt (e.g., pharmaceutically acceptable salt) thereof.
B31. The compound of embodiment B11, wherein the compound is a compound according to Formula IIV, or a salt (e.g., pharmaceutically acceptable salt) thereof.
B32. The compound of embodiment B11, wherein the compound is a compound according to Formula IIW, or a salt (e.g., pharmaceutically acceptable salt) thereof.
B33. The compound of embodiment B11, wherein the compound is a compound according to Formula IIX, or a salt (e.g., pharmaceutically acceptable salt) thereof.
B34 The compound of embodiment B11, wherein the compound is a compound according to Formula IIY, or a salt (e.g., pharmaceutically acceptable salt) thereof.
B35. The compound of embodiment B11, wherein the compound is a compound according to Formula IIZ, or a salt (e.g., pharmaceutically acceptable salt) thereof.
B36. The compound of any one of embodiments BJ-B35, wherein R⁶ is a monocyclic heteroaryl that is substituted with one or more R¹⁵.
B37. The compound of embodiment B36, wherein R⁶ is a pyridine substituted with one or more R¹⁵.
B38. The compound of embodiment B37, wherein R⁶ has the structure

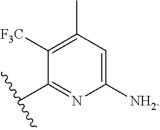 [SMILES: *c1nc(N)cc(C)c1C(F)(F)F]

B39. The compound of any one of embodiments B1-B35, wherein R¹ is a bicyclic heteroaryl that is substituted with one or more R¹³.
B40. The compound of embodiment B39, wherein R⁶ is selected from:

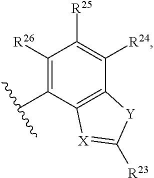

wherein:
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, deuterium, halogen, and C₁₋₆alkyl, wherein any C₁₋₆alkyl is unsubstituted or substituted with one or more R¹³.
B41. The compound of embodiment B39 or B40, wherein R¹ is selected from:

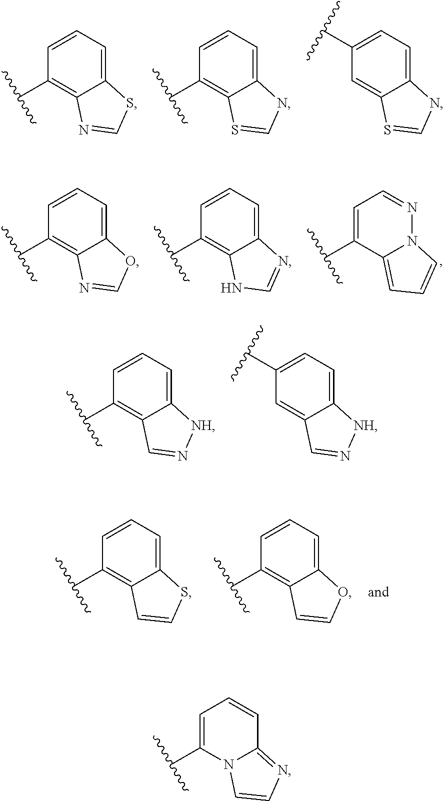

any of which is substituted with one or more R¹³.
B42. The compound of any one of embodiments B39-B41, wherein R⁶ is selected from:

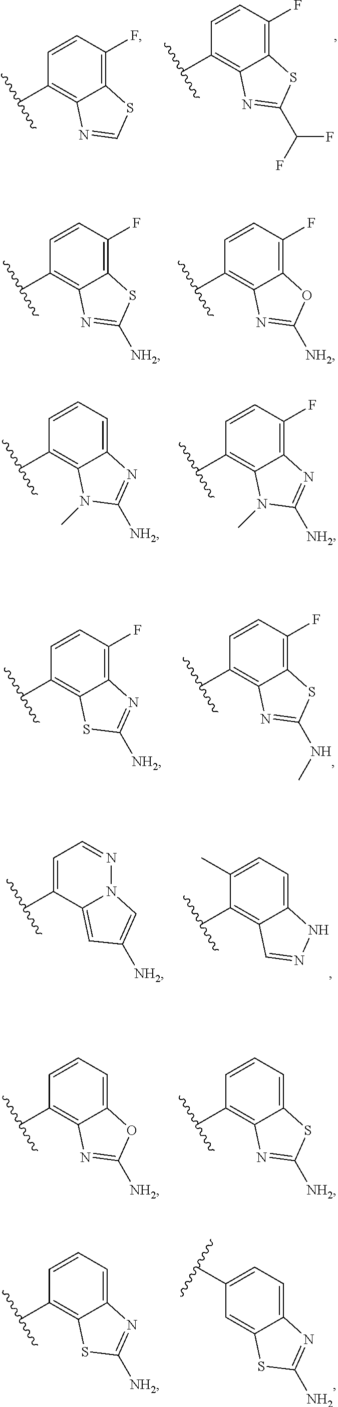

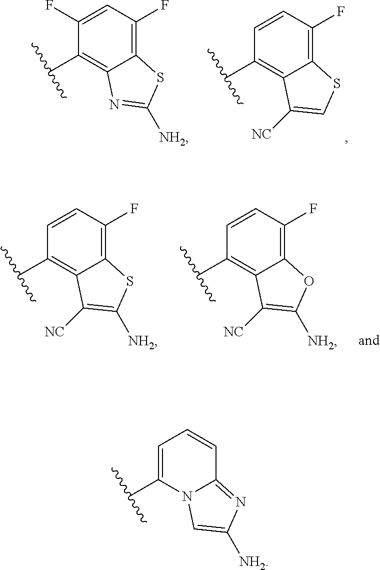 [SMILES: *c1c(F)cc(F)c2sc(N)nc12 ; *c1ccc(F)c2scc(C#N)c12 ; *c1ccc(F)c2sc(N)c(C#N)c12 ; *c1ccc(F)c2oc(N)c(C#N)c12 ; *c1cccc2nc(N)cn12]

B43. The compound of any one of embodiments B39-B42 wherein R¹ is selected from:

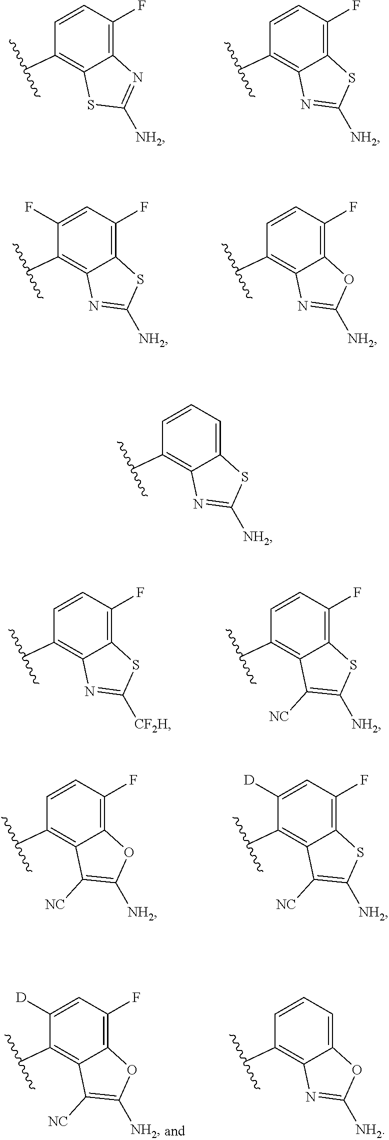

B44. The compound of any one of embodiments BI—B43, wherein the compound is a compound according to Formula IIA1, IIB1, IIC1, IID1, IIE1, IIF1, IIG1, IIH1, IIJ1, IIK1, IIL1, IIM1, IIN1, IIP1, IIQ1, IIR1, IIS1, IIT1, IIU1, IIV1, IIW1, IIX1, IIY1, or IIZ1:

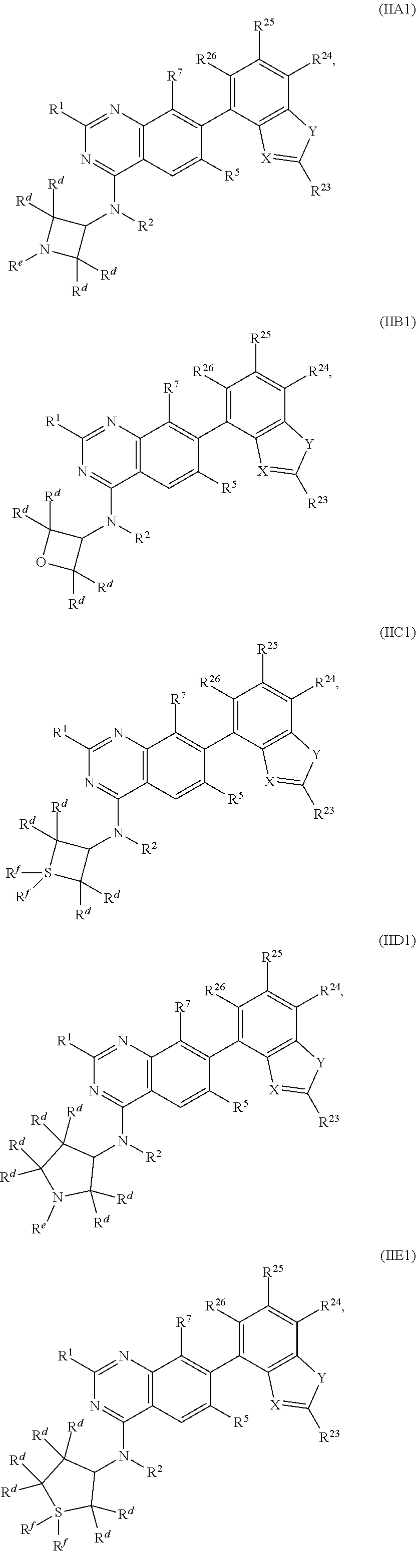

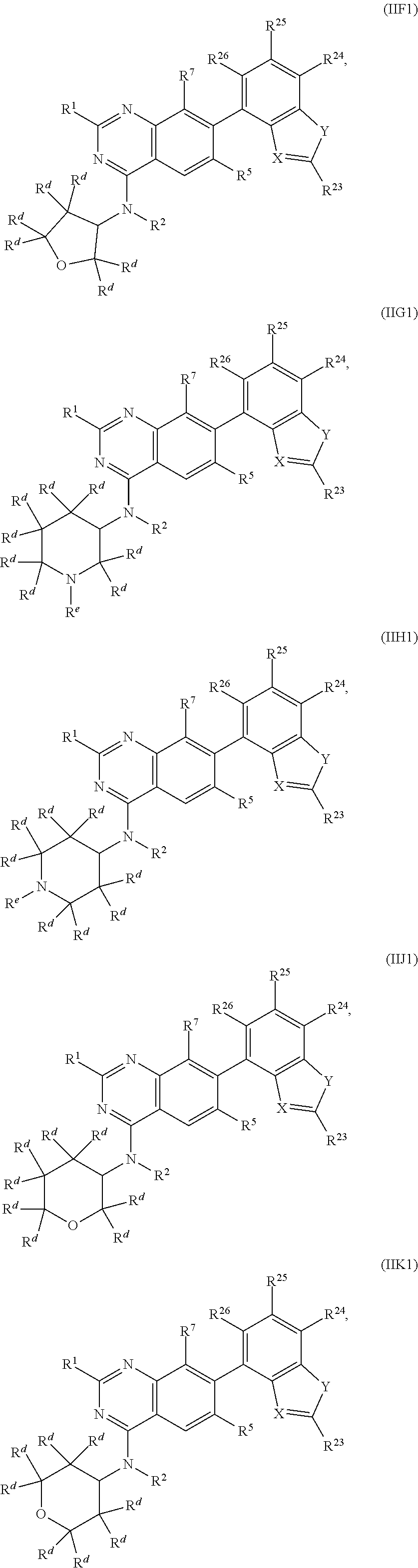

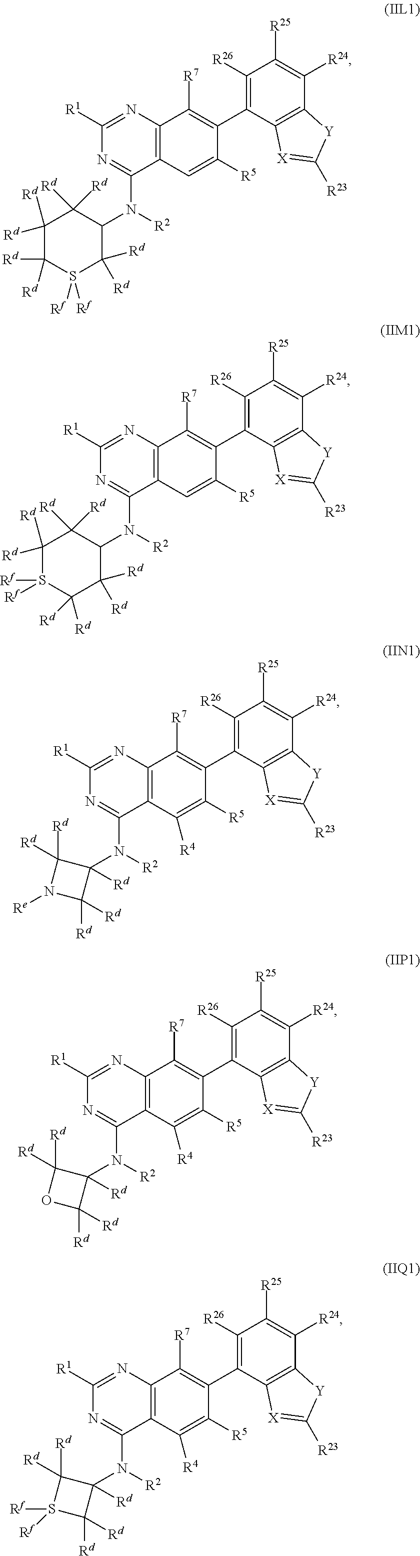

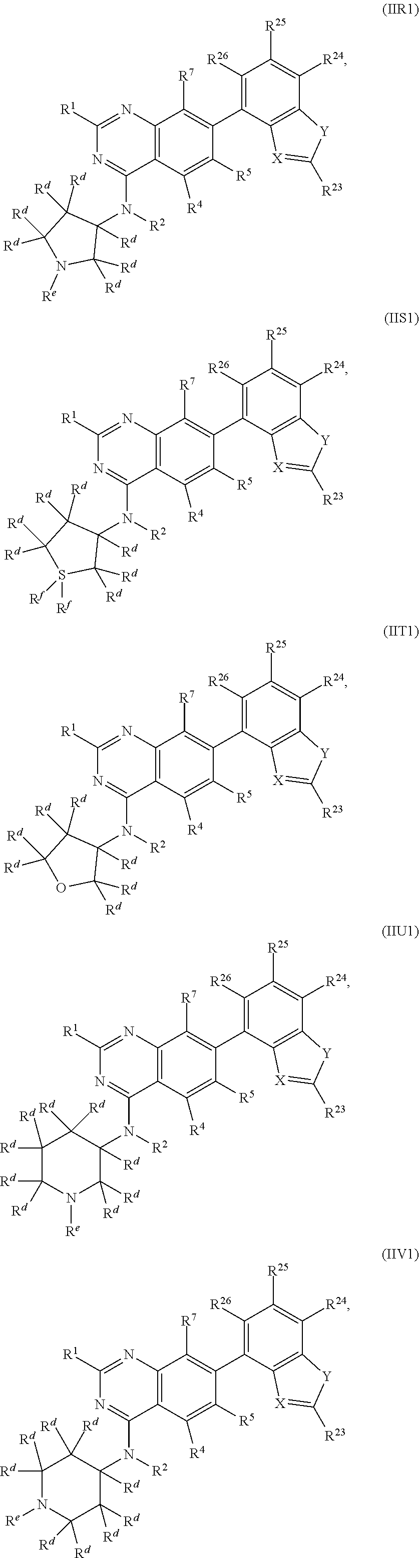

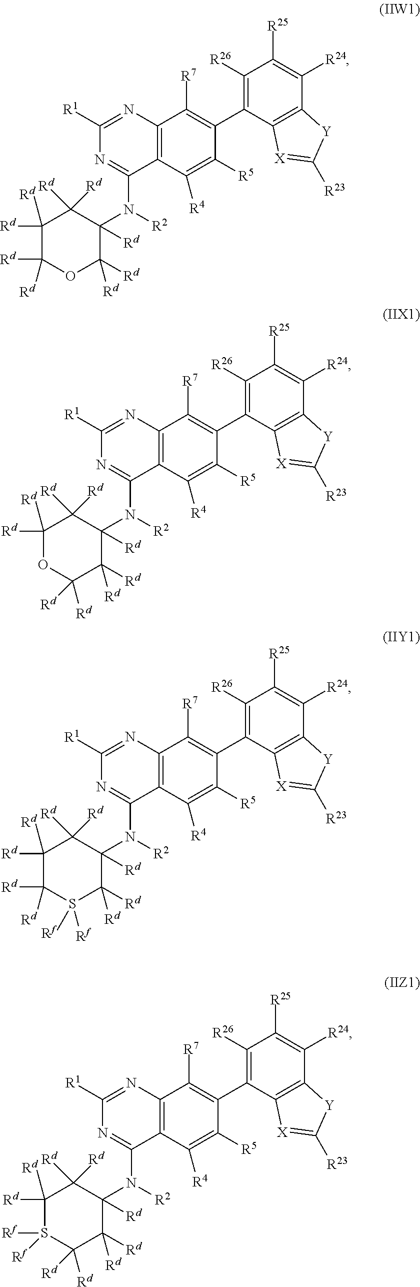

or a salt (e.g., pharmaceutically acceptable salt) thereof, wherein:
each R^{d} is independently selected from H, —OR¹², ═O, —C(O)(C₁₋₆alkylene)CN, —C(O)(C₁₋₆ alkylene)OH, —C(O)(C₁₋₆alkyl), —C(O)N(R¹⁴)₂, —C(O)(3-6 membered carbocycle), —S(O)₂(C₁₋₆alkyl), halogen, and C₁₋₆alkyl, wherein any C₁₋₆alkyl is unsubstituted or substituted with one or more R²⁰;
R^{e}, if present, is selected from —C(O)(C₁₋₆alkylene)CN, —C(O)(C₁₋₆alkylene)OH, —C(O)(C₁₋₆alkyl), —C(O)N(R¹⁴)₂, —C(O)OR¹⁴, —C(O)(3-6 membered carbocycle), —S(O)₂(C₁₋₆alkyl), and C₁₋₆alkyl, wherein any C₁₋₆alkyl is unsubstituted or substituted with one or more R²⁰, and wherein any 3-6 membered carbocycle is unsubstituted or substituted with one or more R¹² or R²⁰;
each R^{f} is optionally absent and, if present, is selected from ═O, —NH₂, —NHC₁₋₆alkyl, and —N(C₁₋₆alkyl)₂;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, deuterium, halogen, and C₁₋₆alkyl, wherein any C₁₋₆alkyl is unsubstituted or substituted with one or more R¹³.
B45. The compound of embodiment B44, wherein the compound is a compound according to Formula IIA1, or a salt (e.g., pharmaceutically acceptable salt) thereof.
B46. The compound of embodiment B44, wherein the compound is a compound according to Formula IIB1, or a salt (e.g., pharmaceutically acceptable salt) thereof.
B47. The compound of embodiment B44, wherein the compound is a compound according to Formula IIC1, or a salt (e.g., pharmaceutically acceptable salt) thereof.
B48. The compound of embodiment B44, wherein the compound is a compound according to Formula IID1, or a salt (e.g., pharmaceutically acceptable salt) thereof.
B49. The compound of embodiment B44, wherein the compound is a compound according to Formula IIE1, or a salt (e.g., pharmaceutically acceptable salt) thereof.
B50 The compound of embodiment B44, wherein the compound is a compound according to Formula IIF1, or a salt (e.g., pharmaceutically acceptable salt) thereof.
B51. The compound of embodiment B44, wherein the compound is a compound according to Formula IIG1, or a salt (e.g., pharmaceutically acceptable salt) thereof.
B52. The compound of embodiment B44, wherein the compound is a compound according to Formula IIH1, or a salt (e.g., pharmaceutically acceptable salt) thereof.
B53. The compound of embodiment B44, wherein the compound is a compound according to Formula IIJ1, or a salt (e.g., pharmaceutically acceptable salt) thereof.
B54. The compound of embodiment B44, wherein the compound is a compound according to Formula IIK1, or a salt (e.g., pharmaceutically acceptable salt) thereof.
B55. The compound of embodiment B44, wherein the compound is a compound according to Formula IIL1, or a salt (e.g., pharmaceutically acceptable salt) thereof.
B56. The compound of embodiment B44, wherein the compound is a compound according to Formula IIM1, or a salt (e.g., pharmaceutically acceptable salt) thereof.
B57. The compound of embodiment B44, wherein the compound is a compound according to Formula IIN1, or a salt (e.g., pharmaceutically acceptable salt) thereof.
B58. The compound of embodiment B44, wherein the compound is a compound according to Formula IIP1, or a salt (e.g., pharmaceutically acceptable salt) thereof.
B59. The compound of embodiment B44, wherein the compound is a compound according to Formula IIQ1, or a salt (e.g., pharmaceutically acceptable salt) thereof.
B60. The compound of embodiment B44, wherein the compound is a compound according to Formula IIR1, or a salt (e.g., pharmaceutically acceptable salt) thereof.
B61. The compound of embodiment B44, wherein the compound is a compound according to Formula IIS1, or a salt (e.g., pharmaceutically acceptable salt) thereof.
B62. The compound of embodiment B44, wherein the compound is a compound according to Formula IIT1, or a salt (e.g., pharmaceutically acceptable salt) thereof.
B63. The compound of embodiment B44, wherein the compound is a compound according to Formula IIU1, or a salt (e.g., pharmaceutically acceptable salt) thereof.
B64. The compound of embodiment B44, wherein the compound is a compound according to Formula IIV1, or a salt (e.g., pharmaceutically acceptable salt) thereof.
B65. The compound of embodiment B44, wherein the compound is a compound according to Formula IIW1, or a salt (e.g., pharmaceutically acceptable salt) thereof.
B66. The compound of embodiment B44, wherein the compound is a compound according to Formula IIX1, or a salt (e.g., pharmaceutically acceptable salt) thereof.
B67 The compound of embodiment B44, wherein the compound is a compound according to Formula IIY1, or a salt (e.g., pharmaceutically acceptable salt) thereof.
B68. The compound of embodiment B44, wherein the compound is a compound according to Formula IIZ1, or a salt (e.g., pharmaceutically acceptable salt) thereof.
B69. The compound of any one of embodiments B44-1368, wherein X is C—CN, Y is S, and R² is —N(R¹²)₂.
B70. The compound of any one of embodiments B44-B69, wherein one or more of R²⁴, R²⁵, and R²⁶ is a halogen (e.g., F).
B71. The compound of any one of embodiments B14, B16, B22, B23, B26, B28, B34, B35, B47, B49, B55, B56, B59, B61, B67, and B68, wherein each R^{f} is ═O.
B72. The compound of any one of embodiments B14, B16, B22, B23, B26, B28, B34, B35, B47, B49, B55, B56, B59, B61, B67, and B68, wherein each R^{f} is absent.
B73. The compound of any one of embodiments B12, B15, B18, B19, B24, B27, B30, B31, B45, B48, B51, B52, B57, B60, B63, and B64, wherein R is C₁₋₆ alkyl that is unsubstituted or substituted with one or more R²⁰.
B74. The compound of any one of embodiments B12, B15, B18, B19, B24, B27, B30, B31, B45, B48, B51, B52, B57, B60, B63, and B64, wherein R^{e} is —C(O)(3-6 membered carbocycle).
B75. The compound of any one of embodiments B12-B74, wherein each R^{d} is H.
B76. The compound of any one of embodiments B12-B74, wherein at least one R^{d} is selected from —OR¹² and a C₁₋₆alkyl substituted with —OH.
B77. The compound of any one of embodiments B1-B76, wherein R² is H.
B78. The compound of any one of embodiments B1-B76, wherein R² is selected from C₁₋₆alkyl that is unsubstituted or is substituted with one or more R¹³.
B79. The compound of embodiment B78, wherein R² is selected from C₁₋₂alkyl.
B80. The compound of any one of embodiments B1-B79, wherein R¹ is selected from —OR⁸.
B81. The compound of embodiment B80, wherein R¹ is selected from:

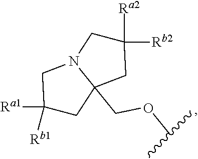

wherein R^{a1}, R^{a2}, R^{b1}, and R^{b2} are each independently selected from deuterium, halogen, C₁₋₆alkyl, —OR¹², and H, wherein R^{a2} and R^{b2} can optionally join together to form a 3-6 membered carbocycle, and wherein any C₁₋₆alkyl or 3-6 membered carbocycle is unsubstituted or is substituted with one or more R¹³.
B82. The compound of embodiment B81, wherein R¹ is selected from:

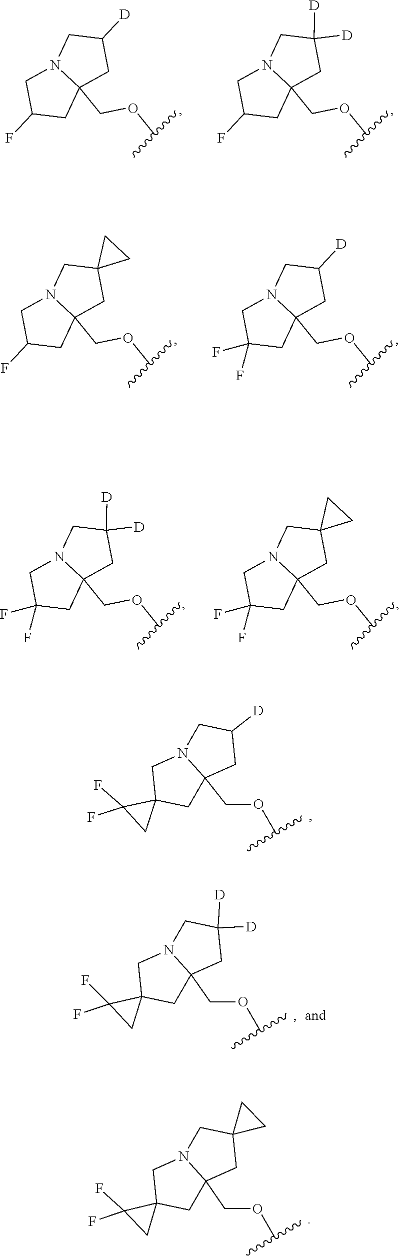

B83. The compound of embodiment B80, wherein R¹ is selected from:

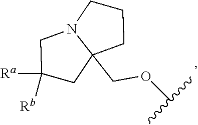

wherein R^{a} and R^{b} are each independently selected from halogen, C₁₋₆alkyl, —OR¹², and H, wherein any C_{1-b}alkyl is unsubstituted or is substituted with one or more R¹³.

B84. The compound of embodiment B83, wherein R¹ is selected from:

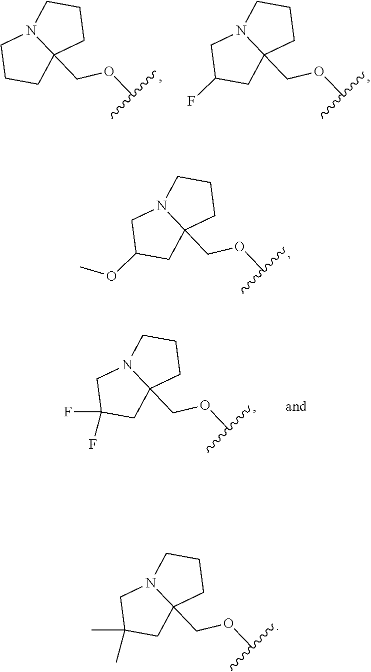 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

B85. The compound of embodiment B80, wherein R¹ is selected from:

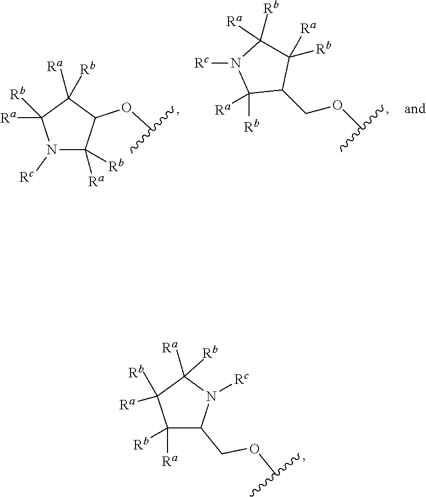

wherein each R^{a} and R^{b} is independently selected from halogen, C₁₋₆ alkyl, —OR¹², and H; and R^{c} is selected from C₁₋₆ alkyl, wherein an R^{a} and R^{b} or R optionally join together to form a 3-6 membered carbocycle or heterocycle, and wherein any C₁₋₆ alkyl or 3-6 membered carbocycle or heterocycle is unsubstituted or is substituted with one or more R¹³.
B86. The compound of embodiment B85, wherein R is selected from:

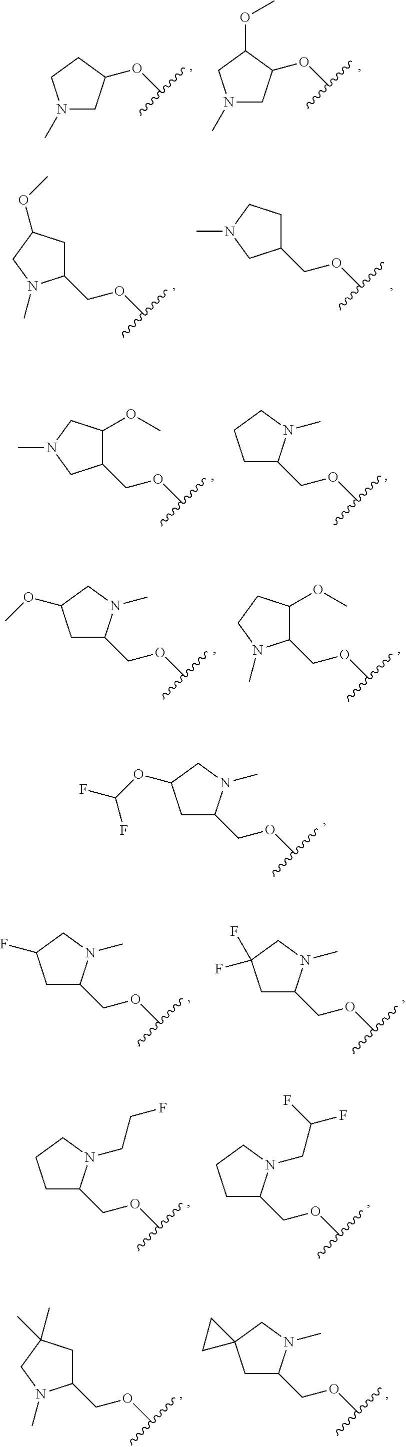

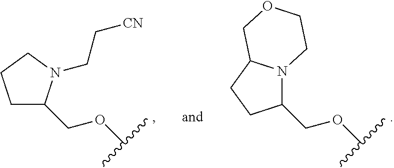 [SMILES: *OCC1CCCN1CCC#N ; *OCC1CCC2COCCN12]

B87. The compound of any one of embodiments B1-879, wherein R¹ is selected from

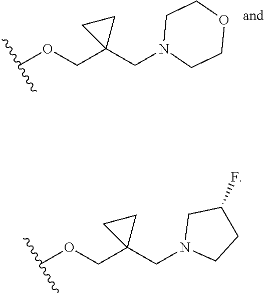 [SMILES: *OCC1(CN2CCOCC2)CC1 ; *OCC1(CN2CC[C@@H](F)C2)CC1]

B88. The compound of any one of embodiments B81-B87, wherein R⁵ is H.
B89. The compound of any one of embodiments B1-B87, wherein R¹ is a halogen (e.g., F or Cl).
B90. The compound of any one of embodiments B1-B87, wherein R⁵ is selected from C₁₋₆alkyl that is unsubstituted or substituted with one or more R¹³.
B91. The compound of embodiment B90, wherein R⁵ is selected from C₁₋₆alkyl that is substituted with one or more halogens or —CN.
B92. The compound of embodiment B91, wherein R⁵ is selected from —CF₂H, —CF₃, —CH₂CN, and —CH₂CH₃.
B93. The compound of embodiment B92, wherein R⁵ is —CF₃.
B94. The compound of any one of embodiments B1-B93, wherein the compound is a not a compound included in Table 1.
B95. A compound represented by Formula III′:

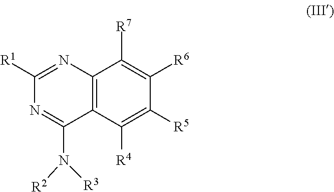

or a salt (e.g., pharmaceutically acceptable salt) thereof, wherein:

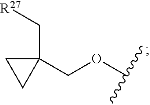

R¹ is selected from —OR⁸ and;
R² and R³, together with the nitrogen atom to which they are attached, form a 4-10 membered heterocycle that is unsubstituted or is substituted with one or more R¹³;
R⁴ is H;
R⁵ is selected from halogen and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹¹ is independently selected from deuterium, —OR¹², ═O, =N(R¹⁴), —C(O)(C₁₋₆alkylene)CN, —C(O)(C₁₋₆alkylene)OR¹², —C(O)(C₁₋₆alkyl), —C(O)N(R)₂, —C(O)(3-6 membered carbocycle or heterocycle), —S(O)₂(C₁₋₆alkyl), —N(R¹⁴)C(O)(C₁₋₆alkyl), —N(R¹⁴)C(O)(C₁₋₆alkylene)OR¹⁴, halogen, —CN, a 3-6 membered carbocycle or heterocycle, and C₁₋₆alkyl, wherein any C₁₋₆alkyl, carbocycle, or heterocycle is unsubstituted or substituted with one or more R²;
each R² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹³;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen; and
R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.
B96. A compound represented by Formula III:

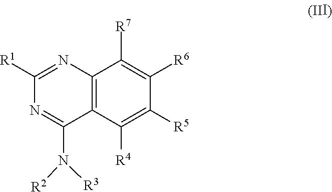

or a salt (e.g., pharmaceutically acceptable salt) thereof, wherein:
R¹ is selected from —OR⁸ and

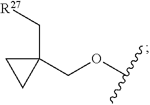

R² and R³, together with the nitrogen atom to which they are attached, form a 4-10 membered heterocycle that is unsubstituted or is substituted with one or more R¹¹, provided that when the heterocycle contains an additional nitrogen atom, the additional nitrogen atom is substituted with R¹³;
   R⁴ is H;
   R⁵ is selected from halogen and C₁₋₆alkyl, wherein any C₁₋₆alkyl is unsubstituted or substituted with one or more R¹³;
   R⁶ is a monocyclic or bicyclic heteroaryl, wherein the heteroaryl is substituted with one or more R¹⁵;
   R⁷ is selected from halogen;
   R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
   each R¹¹ is independently selected from deuterium, —OR¹², ═O, =N(R¹⁴), —C(O)(C₁₋₆alkylene)CN, —C(O)(C₁₋₆alkylene)OR¹², —C(O)(C₁₋₆alkyl), —C(O)N(R¹⁴)₂, —C(O)(3-6 membered carbocycle or heterocycle), —S(O)₂(C₁₋₆alkyl), —N(R¹⁴)C(O)(C₁₋₆alkyl), —N(R¹⁴)C(O)(C₁₋₆alkylene)OR¹², halogen, —CN, a 3-6 membered carbocycle or heterocycle, and C₁₋₆alkyl, wherein any C₁₋₆alkyl, carbocycle, or heterocycle is unsubstituted or substituted with one or more R²⁰;
   each R¹² is independently selected from C₁₋₆ alkyl, C₂₋₆ alkenyl, and H, wherein any C₁₋₆akyl or C₂₋₆ alkenyl is unsubstituted or substituted with one or more R¹³;
   each R¹³ is independently selected from —OR¹⁴, —CN, —N(R¹⁴)₂, and halogen;
   each R¹⁴ is independently selected from C₁₋₆alkyl, C₂₋₆ alkenyl, and H;
   each R¹⁵ is independently selected from deuterium, halogen, —N(R¹²)₂, —CN, and C₁₋₆alkyl, wherein any C₁₋₆alkyl is unsubstituted or substituted with one or more R¹³;
   each R²⁰ is independently selected from —OH, —OC₁₋₆alkyl, —CN, —NH₂, —NHC₁₋₆alkyl, and halogen;
   R²⁷ is a 3-6 membered heterocycle including one or more heteroatoms selected from N, O, and S, wherein the heterocycle is unsubstituted or substituted with one or more R¹³;
   each R²⁸ is independently selected from C₁₋₆alkyl and halogen; and
   R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.
B97. The compound of embodiment B95 or B96, wherein R² and R³, together with the nitrogen atom to which they are attached, form a 4-7 membered heterocycle.
B98. The compound of embodiment B97, wherein R² and R³, together with the nitrogen atom to which they are attached, form a 4-7 membered heterocycle that includes 1 or 2 heteroatoms selected from O, S, and N.
B99. The compound of embodiment B98, wherein R² and R³, together with the nitrogen atom to which they are attached, form a 4-7 membered heterocycle that includes 1 or 2 heteroatoms selected from O, S, and N, wherein the heterocycle is substituted with 1-4 R¹¹.
B100. The compound of any one of embodiments B95-B99, wherein R² and R³, together with the nitrogen atom to which they are attached, form a pyrrolidine that is substituted with 0-4 R¹¹.
B101. The compound of any one of embodiments B95-899, wherein R² and R³, together with the nitrogen atom to which they are attached, form an azetidine that is substituted with 0-4 R¹¹.
B102. The compound of any one of embodiments B95-B99, wherein R² and R³, together with the nitrogen atom to which they are attached, form a piperidine that is substituted with 0-4 R¹¹.
B103. The compound of any one of embodiments B95-B99, wherein R² and R³, together with the nitrogen atom to which they are attached, form a piperazine that is substituted with 0-4 R¹¹.
B104. The compound of any one of embodiments B95-B99, wherein R² and R³, together with the nitrogen atom to which they are attached, form a morpholine or thiomorpholine that is substituted with 0-4 R¹¹.
B105. The compound of any one of embodiments B95-B99, wherein R² and R³, together with the nitrogen atom to which they are attached, form a 7-membered heterocycle that includes 1-3 heteroatoms selected from O, S, and N.
B106. The compound of any one of embodiments B95-B105, wherein R² and R³, together with the nitrogen atom to which they are attached, form a 4-7 membered heterocycle substituted with one or more R¹¹, wherein at least one R¹¹ is selected from —OR¹² and a C₁₋₆alkyl substituted with —OH.
B107. The compound of any one of embodiments B95-B106, wherein R² and R³, together with the nitrogen atom to which they are attached, form a 4-7 membered heterocycle substituted with one or more R¹¹, wherein at least one R¹¹ is an unsubstituted C₁₋₆alkyl.
B108. The compound of any one of embodiments B95-B105, wherein R² and R³, together with the nitrogen atom to which they are attached, form a 4-7 membered heterocycle that is unsubstituted.
B109. The compound of embodiment B95 or B96, wherein R² and R³, together with the nitrogen atom to which they are attached, form a bridged heterocycle.
B110. The compound of embodiment B109, wherein R² and R³, together with the nitrogen atom to which they are attached, form a 7-9 membered bridged heterocycle that includes 1 or 2 heteroatoms selected from O, S, and N.
B111. The compound of embodiment B110, wherein R² and R³, together with the nitrogen atom to which they are attached, form a 7-9 membered bridged heterocycle that includes 1 or 2 heteroatoms selected from O, S, and N, wherein the bridged heterocycle is substituted with 1-4 R¹¹.
B112. The compound of any one of embodiments B109-B111, wherein R² and R³, together with the nitrogen atom to which they are attached, form a bridged piperidine that is substituted with 0-4 R¹¹.
B113. The compound of any one of embodiments B109-B111, wherein R² and R³, together with the nitrogen atom to which they are attached, form a bridged piperazine that is substituted with 0-4 R¹¹.
B114. The compound of any one of embodiments B109-B111, wherein R² and R³, together with the nitrogen atom to which they are attached, form a bridged morpholine or thiomorpholine that is substituted with 0-4 R¹¹.
B115. The compound of any one of embodiments B109-B114, wherein R² and R³, together with the nitrogen atom to which they are attached, form a bridged heterocycle substituted with one or more R¹¹, wherein at least one R¹¹ is selected from —OR¹² and a C₁₋₆alkyl substituted with —OH.
B116. The compound of any one of embodiments B109-B115, wherein R² and R³, together with the nitrogen atom to which they are attached, form a bridged heterocycle substituted with one or more R¹¹, wherein at least one R¹¹ is an unsubstituted C₁₋₆alkyl.
B117. The compound of any one of embodiments B109-B114, wherein R² and R³, together with the nitrogen atom to which they are attached, form a bridged heterocycle that is unsubstituted.
B118. The compound of embodiment B95 or B96, wherein R² and R³, together with the nitrogen atom to which they are attached, form a spirocycle.
B119. The compound of embodiment B95 or B96, wherein R² and R³, together with the nitrogen atom to which they are attached, form a fused ring system including at least two rings.
120. The compound of any one of embodiments B95-B107, B109-B116, B118, and B119, wherein R² and R³, together with the nitrogen atom to which they are attached, form a 4-10 membered heterocycle that is substituted with one or more R¹¹, wherein the one or more R¹¹ are independently selected from deuterium, —OR¹², ═O, =N(R¹⁴), —C(O)(C₁₋₆alkylene)CN, —C(O)C₁₋₆alkylene)OR¹⁴, —C(O)(C_{1-b}alkyl), —C(O)N(R¹⁴)₂, —S(O)₂(C₁₋₆alkyl), —N(R¹⁴)C(O)(C₁₋₆alkyl), —N(R¹⁴)C(O)(C₁₋₆alkylene)OR¹², halogen, and C₁₋₆alkyl that is unsubstituted or substituted with one or more R²⁰.
B121. The compound of embodiment B120, wherein R² and R³, together with the nitrogen atom to which they are attached, form a 4-10 membered heterocycle that is substituted with one or more R¹¹, wherein the one or more R¹¹ are independently selected from —OR¹², —C(O)(C₁₋₆alkylene)CN, —C(O)(C₁₋₆alkylene)OR¹⁴, —C(O)(C₁₋₆alkyl), —C(O)N(R¹⁴)₂, —N(R¹⁴)C(O)(C₁₋₆alkyl), —N(R¹⁴)C(O)(C₁₋₆alkylene)OR¹², halogen, and C₁₋₆alkyl that is unsubstituted or substituted with one or more R²⁰.
B122. The compound of any one of embodiments B95-B121, wherein R² and R³, together with the nitrogen atom to which they are attached, form a structure selected from:

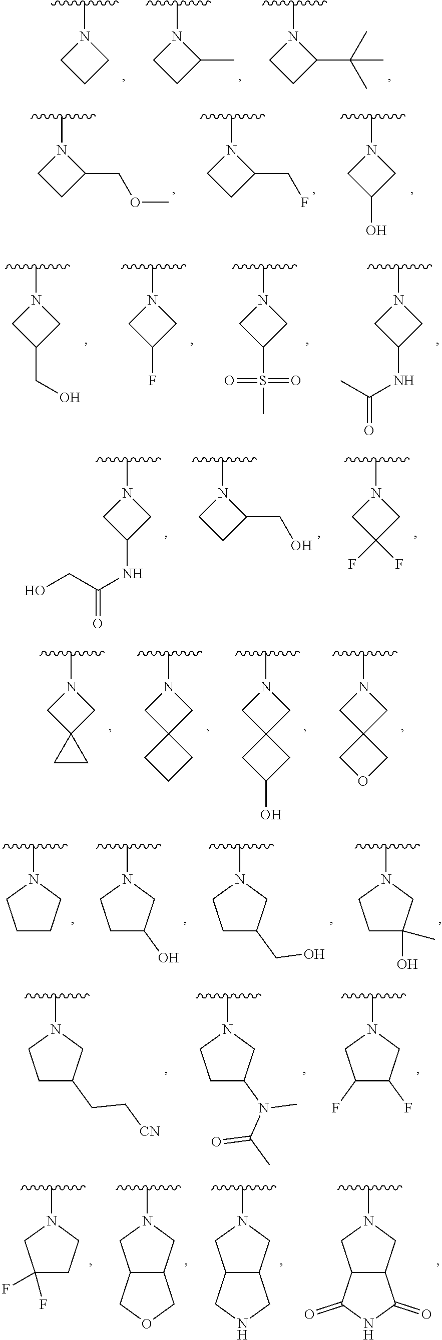

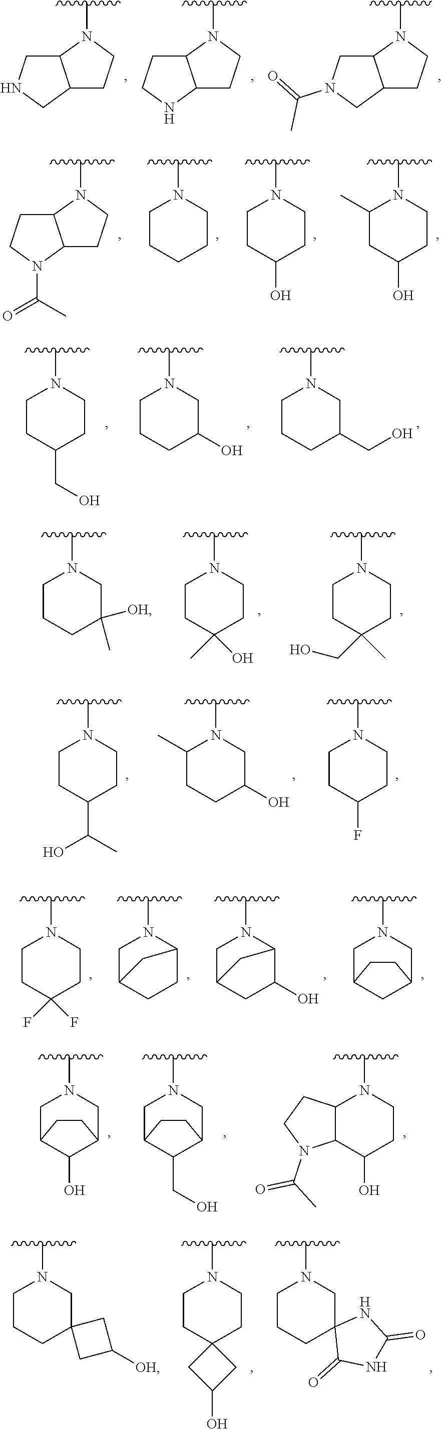

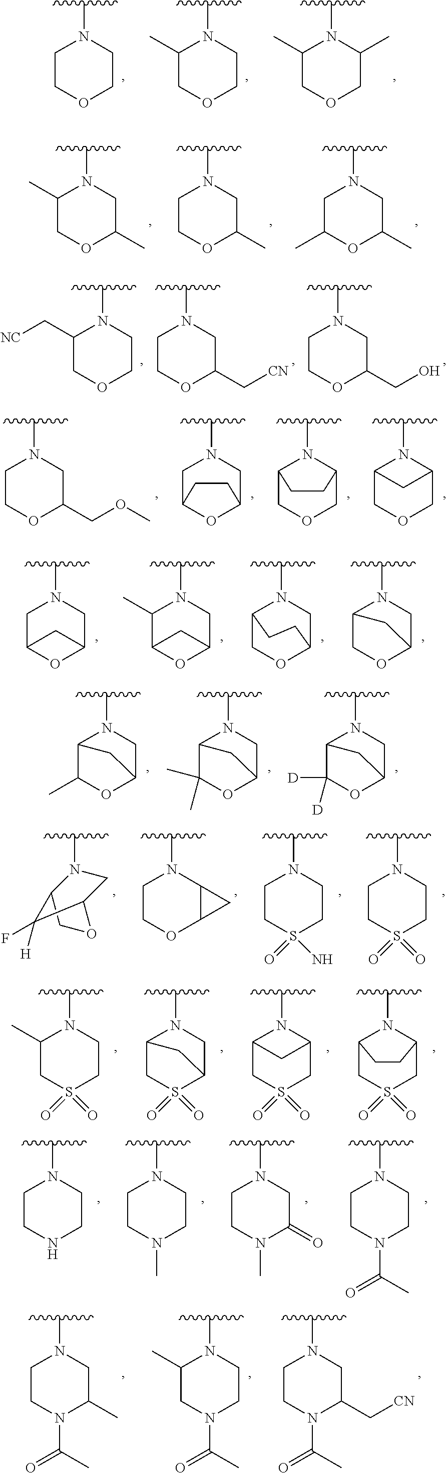

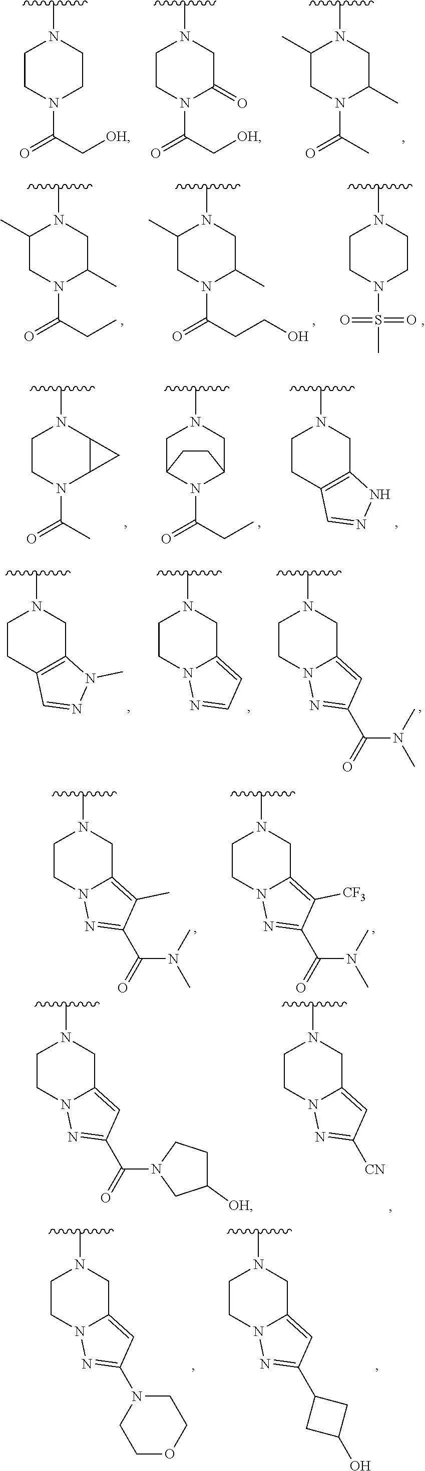

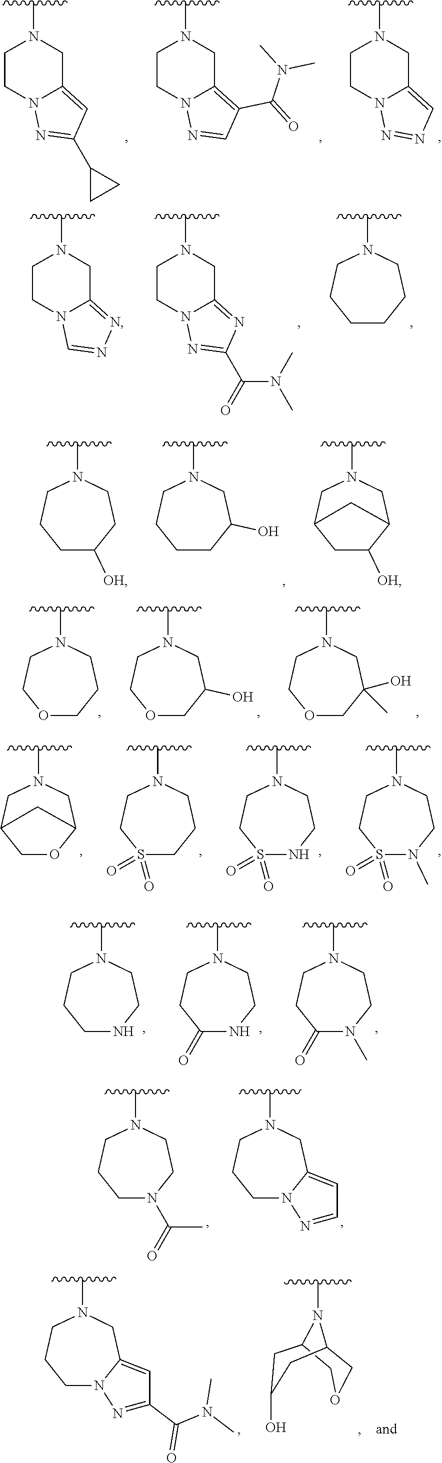

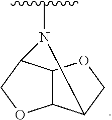 [SMILES: *N1C2COC3C2OCC31]

any of which is optionally further substituted with one or more R¹³.
B123. The compound of any one of embodiments B95-B122, wherein the compound is a compound according to Formula IIIA:

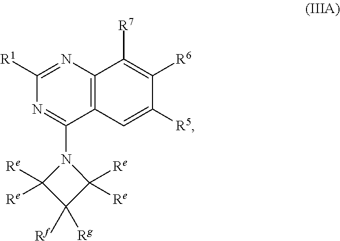

or a salt (e.g., pharmaceutically acceptable salt) thereof, wherein:
each R^{e} is independently selected from deuterium, hydrogen, —OR¹², ═O, =N(R¹⁴), —C(O)(C₁₋₆alkylene)CN, —C(O)(C₁₋₆alkylene)OR¹⁴, —C(O)(C₁₋₆alkyl), —C(O)N(R¹⁴,)₂—S(O)₂(C₁₋₆ alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰; and
R^{f} and R^{g} are each independently selected from hydrogen, deuterium, —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R⁰, or
R^{f} and R^{g} join together to form a 3-6 membered carbocycle or heterocycle that is unsubstituted or substituted with one or more substituents selected from —OR¹², ═O, —C(O)(C_{I}-alkylene)CN, —C(O)(C₁₋₆alkylene)OR¹⁴, —C(O)(C₁₋₆alkyl), —S(O)₂(C₁₋₆alkyl), —N(R¹⁴)C(O)C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰.
B124. The compound of embodiment B123, wherein each R^{e}, R^{f}, and R^{g} is independently selected from hydrogen, —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰.
B125. The compound of embodiment B123, wherein each R^{e}, R^{f}, and R^{g} is independently selected from hydrogen, —OR¹², —C(O)(C₁₋₆alkylene)CN, —C(O)(C₁₋₆alkylene)OR¹², —C(O)(C₁₋₆alkyl), halogen, and C₁₋₆alkyl that is unsubstituted or substituted with one or more R²⁰.
B126. The compound of any one of embodiments B123-B125, wherein each R^{e} is hydrogen.
B127. The compound of any one of embodiments B123-B126, wherein R^{f} and R^{g} join together to form a 3-6 membered carbocycle or heterocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰.
B128. The compound of embodiment B127, wherein R^{f} and R^{g} join together to form a 3-6 membered carbocycle or heterocycle that is unsubstituted.
B129. The compound of embodiment B127, wherein R^{f} and R^{g} join together to form a 3-6 membered carbocycle or heterocycle that is substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O(C₁₋₆alkylene)OR¹⁴, halogen, and C₁₋₆alkyl, wherein any C₁₋₆alkyl is unsubstituted or substituted with one or more R²⁰.
B130. The compound of embodiment B127, wherein R^{f} and R^{g} join together to form a 3-6 membered carbocycle that is unsubstituted or substituted with one or more substituents selected from —OR¹², —O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰.
B131. The compound of embodiment B130, wherein R¹ and R² join together to form a cyclopropane that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰.
B132. The compound of embodiment B127, wherein R^{r} and R^{z} join together to form a 3-6 membered heterocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)C₁₋₆alkylene)OR¹⁴, halogen, and C₁₋₆alkyl, wherein any C₁₋₆ alkyl is unsubstituted or substituted with one or more R²⁰.
B133. The compound of embodiment B132, wherein R^{f} and R^{g} join together to form an oxetane that is unsubstituted or substituted with one or more substituents selected from —OR¹², ═O, —C(O)(C₁₋₆alkylene)CN, —C(O)(C₁₋₆akylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴ halogen, and C₁₋₆alkyl, wherein any C₁₋₆alkyl is unsubstituted or substituted with one or more R²⁰;
B134. The compound of any one of embodiments B95-B122, wherein the compound is a compound according to Formula IIIB:

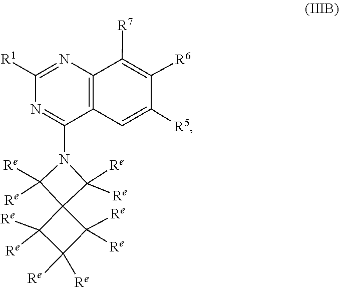

or a salt (e.g., pharmaceutically acceptable salt) thereof, wherein:
each R^{e} is independently selected from hydrogen, deuterium, —OR¹², ═O, =N(R¹⁴), —C(O)(C₁₋₆alkylene)CN, —C(O)(C₁₋₆alkylene)OR¹⁴, —C(O)(C₁₋₆alkyl), —C(O)N(R¹⁴)₂, —S(O)₂(C₁₋₆alkyl), —N(R¹⁴)C(O)(C₁₋₆alkyl), —N(R¹⁴)C(O)(C₁₋₆alkylene)OR¹³, halogen, and C₁₋₆alkyl, wherein any C₁₋₆alkyl is unsubstituted or substituted with one or more R²⁰.
B135. The compound of embodiment B134, wherein each R^{e} is hydrogen.
B136. The compound of any one of embodiments B95-B122, wherein the compound is a compound according to Formula IIIC:

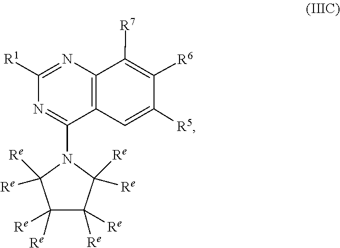

or a salt (e.g., pharmaceutically acceptable salt) thereof, wherein:
each R^{e} is independently selected from hydrogen, deuterium, —OR¹², ═O, =N(R¹⁴), —C(O)(C₁₋₆alkylene)CN, —C(O)(C₁₋₆alkylene)OR¹⁴, —C(O)(C₁₋₆ alkyl), —C(O)N(R¹⁴)₂, —S(O)₂(C₁₋₆ alkyl), —N(R¹⁴)C(O(C₁₋₆alkyl), —N(R¹⁴)C(O)(C₁₋₆ alkylene)OR¹², halogen, and C₁₋₆alkyl, wherein any C₁₋₆alkyl is unsubstituted or substituted with one or more R²⁰.
B137. The compound of embodiment B136, wherein each R^{e} is hydrogen.
B138. The compound of any one of embodiments B95-B122, wherein the compound is a compound according to Formula IIIC1:

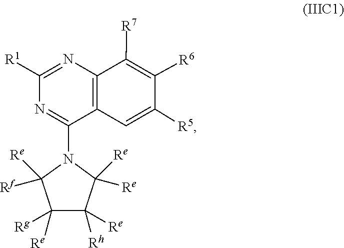

or a salt (e.g., pharmaceutically acceptable salt) thereof, wherein:
each R^{e} is independently selected from hydrogen, deuterium, —OR¹², ═O, =N(R¹⁴), —C(O)(C₁₋₆alkylene)CN, —C(O)(C₁₋₆alkylene)OR¹⁴, —C(O)(C₁₋₆alkyl), —C(O)N(R¹⁴)₂, —S(O)₂(C₁₋₆ akyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R^{e}; and
R^{f}, R^{g}, and R^{h} are each independently selected from hydrogen, deuterium, —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)C₁₋₆alkylene)OR¹⁴, halogen, and C₁₋₆alkyl, wherein any C₁₋₆alkyl is unsubstituted or substituted with one or more R²⁰, optionally wherein
(i) R^{f} and R^{g} join together to form a 3-6 membered carbocycle or heterocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹², halogen, and C₁₋₆alkyl, wherein any C₁₋₆alkyl is unsubstituted or substituted with one or more R²⁰; or
(ii) R^{g} and R^{h} join together to form a 3-6 membered carbocycle or heterocycle that is unsubstituted or substituted with one or more substituents selected from —OR¹², ═O, —C(O)C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰.
B139. The compound of embodiment B138, wherein each R^{e} is hydrogen.
B140. The compound of embodiment B138 or B139, wherein R^{f} and R^{g} join together to form a 3-6 membered carbocycle or heterocycle that is unsubstituted or substituted with one or more substituents selected from —OR¹², ═O, —C(O)C₁₋₆akylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰.
B141. The compound of embodiment B140, wherein R^{f} and R^{g} join together to form a 3-6 membered carbocycle that is unsubstituted or substituted with one or more substituents selected from —OR¹², ═O, —C(O(C₁₋₆alkylene)CN, —C(O)(C₁₋₄alkylene)OR¹⁴, —C(O)(C₁₋₆alkyl), —S(O)₂(C₁₋₆alkyl), —N(R¹⁴)C(O)(C_{I}._alkyl), —N(R¹⁴)C(O)(C₁₋₆alkylene)OR¹⁶, halogen, and C₁₋₆alkyl, wherein any C₁₋₆alkyl is unsubstituted or substituted with one or more R²⁰.
B142. The compound of embodiment B140, wherein R^{f} and R^{g} join together to form a 3-6 membered heterocycle that is unsubstituted or substituted with one or more substituents selected from —OR¹², —O, —C(O)(C₁₋₆alkylene)CN, —C(O)(C₁₋₆alkylene)OR¹², —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹², halogen, and C₁₋₆alkyl, wherein any C₁₋₆ alkyl is unsubstituted or substituted with one or more R²⁰.
B143. The compound of embodiment B138 or B139, wherein R^{g} and R^{h} join together to form a 3-6 membered carbocycle or heterocycle that is unsubstituted or substituted with one or more substituents selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹², halogen, and C₁₋₆alkyl, wherein any C₁₋₆alkyl is unsubstituted or substituted with one or more R¹³.
B144. The compound of embodiment B143, wherein R^{g} and R^{h} join together to form a 3-6 membered carbocycle that is unsubstituted or substituted with one or more substituents selected from —OR¹⁴, ═O, —C(O)(C₁₋₆alkylene)CN, —C(O)(C₁₋₆alkylene)OR¹⁴, —C(O)(C₁₋₆akyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆ alkyl is unsubstituted or substituted with one or more R²⁰.
B145. The compound of embodiment B143, wherein R^{g} and R^{h} join together to form a 3-6 membered heterocycle that is unsubstituted or substituted with one or more substituents selected from —OR¹², ═O, —C(O)(C₁₋₆ alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆ alkyl is unsubstituted or substituted with one or more R²⁰.
B146. The compound of any one of embodiments B95-B122, wherein the compound is a compound according to Formula IIID:

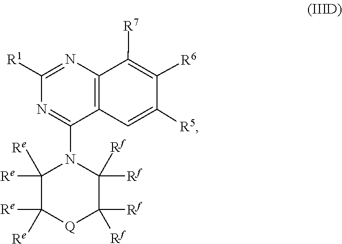

or a salt (e.g., pharmaceutically acceptable salt) thereof, wherein:
Q is selected from CR^{h}R^{j}, NR^{g}, O, S, and SO₂;
each R^{e} and R^{f} is independently selected from R¹¹ and hydrogen, wherein:
   (i) an R^{e} and an R^{f} can optionally join together to form a 4-6 membered ring;
   (ii) a first R^{f} and a second R^{f} connected to adjacent atoms can optionally join together to form a 3-5 membered ring;
   (iii) a first R^{e} and a second R^{e} connected to adjacent atoms can optionally join together to form a 3-5 membered ring; or
   (iv) a first R^{f} and a second R^{f} connected to the same atom can optionally join together to form a 3-5 membered ring,
   wherein any ring formed by one or more R^{e} and/or one or more R^{f} is unsubstituted or substituted with one or more R¹³;
R^{g}, when present, is R¹¹, and
R^{h} and R^{j}, when present, are independently selected from R¹¹ and hydrogen, or can optionally join together to form a 3-4 membered carbocycle or heterocycle that is unsubstituted or substituted with one or more substituents selected from —OR¹², ═O, ═N(R¹⁴), —C(O)(C₁₋₆alkylene)CN, —C(O)(C₁₋₆alkylene)OR¹⁴, —C(O)(C₁₋₆alkyl), —C(O)N(R¹⁴)₂, —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰.
B147. The compound of embodiment B146, wherein Q is NR^{g}.
B148. The compound of embodiment B147, wherein R^{g} is selected from —C(O)(C₁₋₆alkylene)CN, —C(O)(C₁₋₆alkylene)OR¹⁴, —C(O)(C₁₋₆alkyl), —S(O)₂(C₁₋₆alkyl), and C₁₋₆alkyl, wherein any C₁₋₆alkyl is unsubstituted or substituted with one or more R²⁰.
B149. The compound of embodiment B146, wherein Q is O, S, or SO₂.
B150. The compound of embodiment B149, wherein Q is O.
B151. The compound of embodiment B146, wherein Q is CR^{h}R^{j}.
B152. The compound of embodiment B151, wherein the compound is a compound according to Formula IIIE:

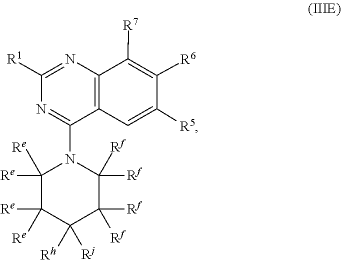

or a salt (e.g., pharmaceutically acceptable salt) thereof.
B153. The compound of embodiment B152, wherein an R^{e} and an R^{f} join together to form a 4-6 membered ring.
B154. The compound of embodiment B152 or B153, wherein R^{h} and R^{j} are independently selected from R¹¹ and hydrogen.
B155. The compound of any one of embodiments B152-B154, wherein R^{h} and/or R^{g} is selected from deuterium, —OR¹², ═O, —C(O)C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₄alkyl is unsubstituted or substituted with one or more R²⁰.
B156. The compound of any one of embodiments B152-B154, wherein R^{h} and R^{j} are each hydrogen.
B157. The compound of embodiment B152, wherein R^{h} and R^{j} join together to form a 3-4 membered carbocycle or heterocycle.
B158. The compound of embodiment B157, wherein R^{h} and R^{j} join together to form a 3-4 membered carbocycle or heterocycle that is unsubstituted.
B159. The compound of embodiment B157, wherein R^{a} and R^{b} join together to form a 3-4 membered carbocycle or heterocycle that is substituted with one or more substituents selected from —OR¹², ═O, —C(O)(C₁₋₆alkylene)CN, —C(O)(C₁₋₆alkylene)OR¹⁴, —C(O)(C₁₋₆akyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl that is unsubstituted or substituted with one or more R²⁰.
B160. The compound of any one of embodiments B157-B159, wherein no combination of one or more R^{e}s and/or one or more R^{f}s join together to form a ring.
B161. The compound of any one of embodiments B157-B160, wherein each R^{e} and R^{f} is hydrogen.
B162. The compound of embodiment B146, wherein the compound is a compound according to Formula IIIF, IIIG, or IIIH:

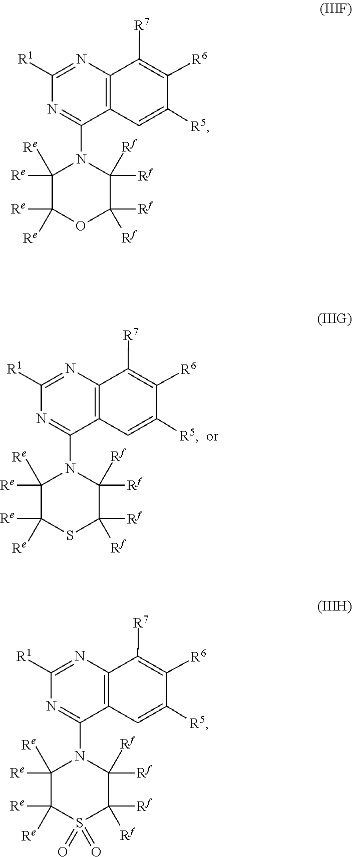

or a salt (e.g., pharmaceutically acceptable salt) thereof.
B163. The compound of embodiment B162, wherein the compound is a compound of Formula IIIF or a salt (e.g., a pharmaceutically acceptable salt) thereof.
B164. The compound of embodiment B162, wherein the compound is a compound of Formula IIIG or a salt (e.g., a pharmaceutically acceptable salt) thereof.
B165. The compound of embodiment B162, wherein the compound is a compound of Formula IIIH or a salt (e.g., a pharmaceutically acceptable salt) thereof.
B166. The compound of any one of embodiments B162-B165, wherein each R^{e} and R^{f} is independently selected from R¹¹ and hydrogen.
B167. The compound of any one of embodiments B162-B165, wherein an R^{e} and an R^{f} join together to form a 4-6 membered ring.
B168. The compound of embodiment B146, wherein the compound is a compound according to Formula IIIJ:

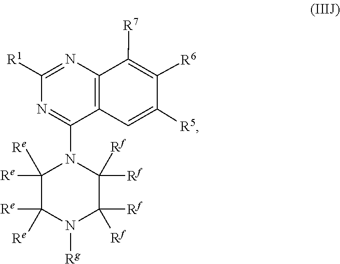

or a salt (e.g., pharmaceutically acceptable salt) thereof.
B169. The compound of embodiment B168, wherein each R^{e} and R^{f} is independently selected from R¹¹ and hydrogen.
B170. The compound of embodiment B168, wherein an R^{e} and an R^{f} join together to form a 4-6 membered ring.
B171. The compound of any one of embodiments B146-B170, wherein the compound is a compound according to Formula IIIK, IIII, IIIM, IIIN, IIIP, IIIQ, or IIIR:

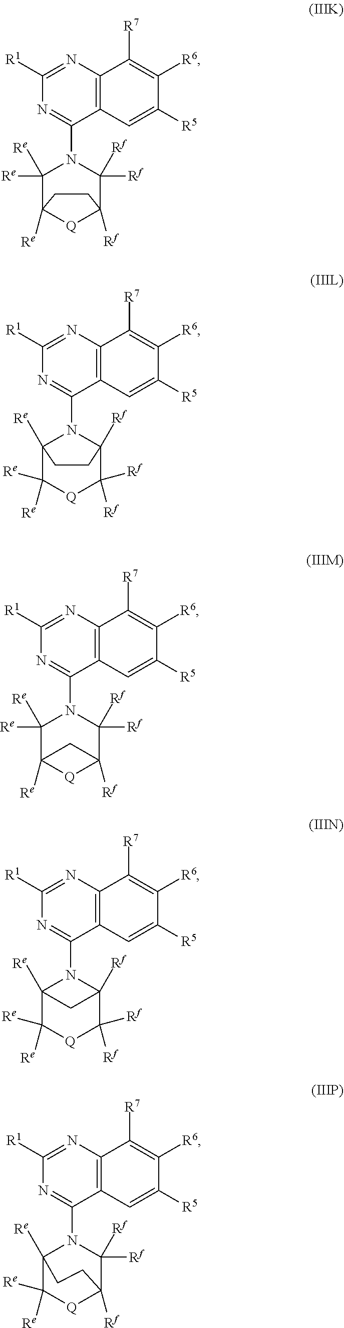

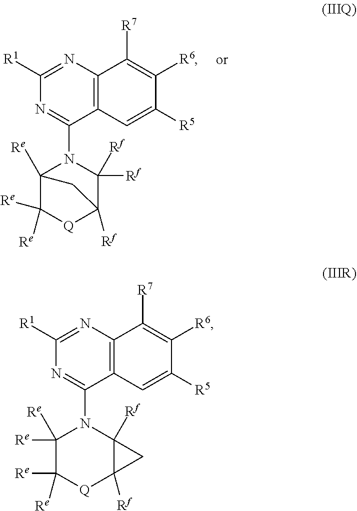

or a salt (e.g., pharmaceutically acceptable salt) wherein:
each R^{e} and R^{f} is independently selected from R¹¹ and hydrogen;
R^{g}, when present, is R¹¹, and
R^{k} and R^{j}, when present, are independently selected from R¹¹ and hydrogen.
B172. The compound of embodiment B171, wherein the compound is a compound of Formula IIIK or a salt (e.g., a pharmaceutically acceptable salt) thereof.
B173. The compound of embodiment B171, wherein the compound is a compound of Formula IIIL or a salt (e.g., a pharmaceutically acceptable salt) thereof.
B174. The compound of embodiment B171, wherein the compound is a compound of Formula HIM or a salt (e.g., a pharmaceutically acceptable salt) thereof.
B175. The compound of embodiment B171, wherein the compound is a compound of Formula IIIN or a salt (e.g., a pharmaceutically acceptable salt) thereof.
B176. The compound of embodiment B171, wherein the compound is a compound of Formula HIP or a salt (e.g., a pharmaceutically acceptable salt) thereof.
B177. The compound of embodiment B171, wherein the compound is a compound of Formula IIIQ or a salt (e.g., a pharmaceutically acceptable salt) thereof.
B178. The compound of embodiment B171, wherein the compound is a compound of Formula IIIR or a salt (e.g., a pharmaceutically acceptable salt) thereof.
B179. The compound of any one of embodiments B171-B178, wherein Q is selected from CR^{h}R^{j}, NR^{g}, and O.
B180. The compound of embodiment B179, wherein Q is CR^{h}R^{j}.
B181. The compound of embodiment B180, wherein R^{h} and R^{j} are independently selected from R¹¹ and hydrogen.
B182. The compound of embodiment B181, wherein R^{e} and/or R^{j} is selected from —OR¹², ═O, =N(R¹⁴), —C(O)C₁₋₆alkylene)CN, —C(O)(C₁₋₆alkylene)OR¹², —C(O)(C₁₋₆alkyl), —C(O)N(R¹⁴)₂, —S(O)₂(C₁₋₆ alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰.
B183. The compound of embodiment B181, wherein R^{h} and R^{j} are each hydrogen.
B184. The compound of embodiment B179, wherein Q is NR^{g}.
B185. The compound of embodiment B184, wherein R^{g} is selected from —C(O)(C₁₋₆alkylene)CN, —C(O)C₁₋₆alkylene)OR¹⁴, —C(O)(C₁₋₆alkyl), —C(O)N(R¹⁴)₂, and C₁₋₆alkyl, wherein any C₁₋₆alkyl is unsubstituted or substituted with one or more R²⁰.
B186. The compound of embodiment B179, wherein Q is O.
B187. The compound of any one of embodiments B171-B178, wherein Q is S or SO₂.
B188. The compound of any one of embodiments B171-B187, wherein each R^{e} and R^{f} is independently selected from R¹¹ and hydrogen.
B189. The compound of embodiment B188, wherein R^{e} and/or R^{f} is selected from deuterium, —OR¹², ═O, =N(R¹⁴), —C(O)(C₁₋₆alkylene)CN, —C(O)(C₁₋₆alkylene)OR¹⁴, —C(O)(C₁₋₆alkyl), —C(O)N(R¹⁴)₂, —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl that is unsubstituted or substituted with one or more R²⁰.
B190. The compound of embodiment B188, wherein each R^{e} and R^{f} is hydrogen.
B191. The compound of any one of embodiments B146-B170, wherein the compound is a compound according to Formula IIIS:

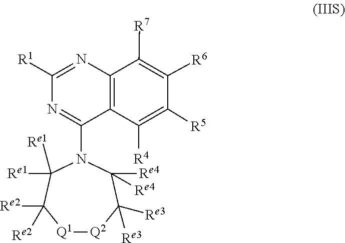

or a salt (e.g., pharmaceutically acceptable salt) wherein:
Q¹ is selected from NR^{g1}, O, SR^{h}₂, and CRⁱR^{j};
Q² is selected from NR^{g2}, O, SR^{h}₂, and CRⁱR^{j};
R^{g1} and R^{g2}, when present, are independently selected from hydrogen, —C(O)(C₁₋₆alkyl), and C₁₋₆alkyl, wherein any C₁₋₆alkyl is unsubstituted or substituted with one or more R²⁰.
each R^{h} is absent or, when present, is independently selected from ═O, =N(R¹⁴), and C₁₋₆alkyl, wherein any C₁₋₆alkyl is unsubstituted or substituted with one or more R²⁰;
each Rⁱ and R^{j}, when present, are independently selected from hydrogen, deuterium, —OR¹², ═O, =N(R¹⁴), —C(O)(C₁₋₆alkylene)CN, —C(O)(C₁₋₆alkylene)OR¹⁴, —C(O)(C₁₋₆alkyl), —C(O)N(R¹⁴)₂. —S(O)₂(C₁₋₄alkyl), —N(R¹⁴)(O)(C₁₋₆alkyl), —N(R¹⁴)C(O)(C₁₋₆alkylene)OR¹³, halogen, and C₁₋₆alkyl, wherein any C₁₋₆alkyl is unsubstituted or substituted with one or more R²⁰; and
each R^{e1}, R^{e2}, R^{e3}, and R^{e4} are independently selected from hydrogen, deuterium, —OR¹², ═O, =N(R¹⁴), —C(O)(C₁₋₆alkylene)CN, —C(O)(C₁₋₆alkylene)OR¹², —C(O)(C₁₋₆alkyl), —C(O)N(R¹⁴)₂, —S(O)₂(C₁₋₆alkyl), —N(R¹⁴)C(O)(C₁₋₆alkyl), —N(R¹⁴)C(O)(C₁₋₆akylene)OR¹⁴, halogen, and C₁₋₆alkyl, wherein any C₁₋₆alkyl is unsubstituted or substituted with one or more R²⁰,
wherein
   (i) an R¹ and an R optionally join together to form a 5-6 membered ring; or
   (ii) when Q² is NR^{g2}, R^{g2} and an R^{e3}, together with the atoms to which they are attached, optionally join together to form a 5-membered heterocycle or heteroaryl that is unsubstituted or substituted with one or more R¹¹, wherein each R¹¹ is independently selected from deuterium, —OR¹², —O, —N(R¹⁴), —C(O)(C₁₋₆alkylene)CN, —C(O)(C₁₋₆alkylene)OR¹⁴, —C(O)(C₁₋₆alkyl), —C(O)N(R¹⁴)₂, —S(O)₂(C₁₋₆akyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰.
B192. The compound of embodiment B191, wherein Q¹ and Q² are each CRⁱR^{j}.
B193. The compound of embodiment B192, wherein at least one R^{e1}, R^{e2}, R^{e3}, or R^{e4} is selected from —OR¹², ═O, =N(R¹⁴), —C(O)(C₁₋₆alkylene)CN, —C(O)(C₁₋₆alkylene)OR¹², —C(O)C₁₋₆alkyl), —C(O)N(R¹⁴)₂, —S(O)₂(C₄alkyl), —N(R¹⁴)C(O)C₁₋₆alkyl), —N(R¹⁴)C(O)(C₁₋₆alkylene)OR¹², halogen, and C₁₋₆alkyl, wherein any C₁₋₆alkyl is unsubstituted or substituted with one or more R¹³.
B194. The compound of embodiment B193, wherein each R^{e1}, R^{e2}, R^{e3}, and R^{e4} is hydrogen.
B195. The compound of embodiment B192, wherein an R² and an R³ join together to form a 5-6 membered ring.
B196. The compound of embodiment B191, wherein Q¹ is CRⁱR^{j} and Q² is NR^{g2}.
B197. The compound of embodiment B196, wherein R^{g2} and an R^{e3}, together with the atoms to which they are attached, join together to form a 5-membered heterocycle or heteroaryl that is unsubstituted or substituted with one or more R¹¹.
B198. The compound of embodiment B196, wherein R¹ is selected from hydrogen, —C(O)(C₁₋₆alkyl), and C₁₋₆alkyl, wherein any C₁₋₆alkyl is unsubstituted or substituted with one or more R²⁰.
B199. The compound of embodiment B191, wherein Q² is CRⁱR^{j} and Q² is NR^{g2}.
B200. The compound of embodiment B199, wherein R^{g2} is selected from hydrogen, —C(O)(C₁₋₆alkyl), and C₁₋₆alkyl, wherein any C₁₋₆alkyl is unsubstituted or substituted with one or more R²⁰.
B201. The compound of embodiment B199 or B200, wherein each R^{e1}, R^{e2}, R^{e3}, and R^{e4} is hydrogen.
B202. The compound of embodiment B191, wherein (i) Q¹ is CRⁱR^{j} and Q² is O or (ii) Q² is CRⁱR^{j} and Q¹ is O.
B203. The compound of embodiment B202, wherein each R^{e1}, R^{e2}, R^{e3}, and R^{e4} is hydrogen.
B204. The compound of embodiment B202, wherein an R^{e2} and an R^{e3} join together to form a 5-6 membered ring.
B205. The compound of embodiment B191, wherein (i) Q¹ is CRⁱR^{j} and Q² is SR^{h}₂ or (ii) Q² is CRⁱR^{j} and Q¹ is SR^{h}₂.
B206. The compound of embodiment B205, wherein each R^{e1}, R^{e2}, R^{e3}, and R^{w4} is hydrogen.
B207. The compound of embodiment B205, wherein an R^{e2} and an R^{e3} join together to form a 5-6 membered ring.
B208. The compound of any one of embodiments B205-B207, wherein each R^{h} is ═O.
B209. The compound of any one of embodiments B192-B208, wherein each Rⁱ and R^{j} are independently selected from hydrogen, deuterium, —OR¹², —O, —N(R¹⁴), —C(O)(C₁₋₆alkylene)CN, —C(O)(C₁₋₆alkylene)OR¹², —C(O)(C₁₋₆alkyl), —C(O)N(R¹⁴)₂, —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R².
B210. The compound of embodiment B209, wherein each R^{h} and R^{j} are hydrogen.
B211. The compound of embodiment B191, wherein (i) NR^{g1} and Q² is SR^{h}₂ or (ii) NR^{g2} and Q¹ is SR^{h}₂.
B212. The compound of embodiment B211, wherein each R^{e1}, R^{e2}, R^{e3}, and R^{e4} is hydrogen.
B213. The compound of embodiment B211, wherein an R^{e2} and an R^{e3} join together to form a 5-6 membered ring.
B214. The compound of any one of embodiments B211-B213, wherein each R^{h} is ═O.
B215. The compound of any one of embodiments B146-B170, wherein the compound is a compound according to Formula IIIT:

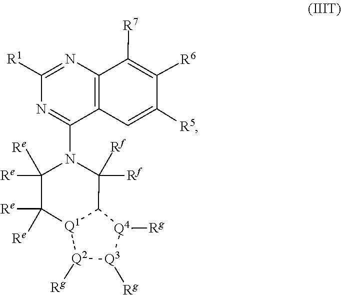

or a salt (e.g., pharmaceutically acceptable salt) wherein:
dashed lines represent single or double bonds, such that the ring containing Q¹, Q², Q³, and Q⁴ is aromatic:
Q¹, Q², Q³, and Q⁴ are independently selected from C and N, wherein at least one of Q¹, Q², Q³, and Q⁴ is N:
each R^{e} and R^{f} is independently selected from R¹¹ and hydrogen; and
each R^{g} is absent or is independently selected from R¹¹ and hydrogen.
B216. The compound of embodiment B215, wherein Q¹ is C.
B217. The compound of embodiment B216, wherein Q² is C, Q³ is N, and Q⁴ is N.
B218. The compound of embodiment B215, wherein Q¹ is N.
B219. The compound of embodiment B218, wherein Q² is N, and Q¹ and Q⁴ are C.
B220. The compound of embodiment B218, wherein Q² and Q³ are N, and Q⁴ is C.
B221. The compound of embodiment B218, wherein Q³ and Q⁴ are N, and Q² is C.
B222. The compound of embodiment B218, wherein Q² and Q⁴ are N, and Q³ is C.
B223. The compound of any one of embodiments B215-B222, wherein each R⁵ is hydrogen or is absent.
B224. The compound of any one of embodiments B95-B223, wherein R⁶ is a bicyclic heteroaryl that is substituted with one or more R¹⁵.
B225. The compound of embodiment B224, wherein R⁶ is selected from

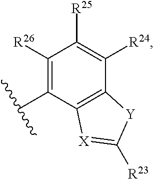

wherein:
   X is selected from N and C—CN;
   Y is selected from O and S;
   R² is selected from —N(R¹²)₂, C₁₋₆alkyl, and C₁₋₆alkyl-N(R¹⁴)₂, wherein any C₁₋₆alkyl is unsubstituted or substituted with one or more R¹³; and
   R²⁴, R²⁵, and R²⁶ are independently selected from H, deuterium, halogen, and C₁₋₆alkyl, wherein any C₁₋₆alkyl is unsubstituted or substituted with one or more R¹³.
B226. The compound of embodiment B224 or B225, wherein R⁶ is selected from:

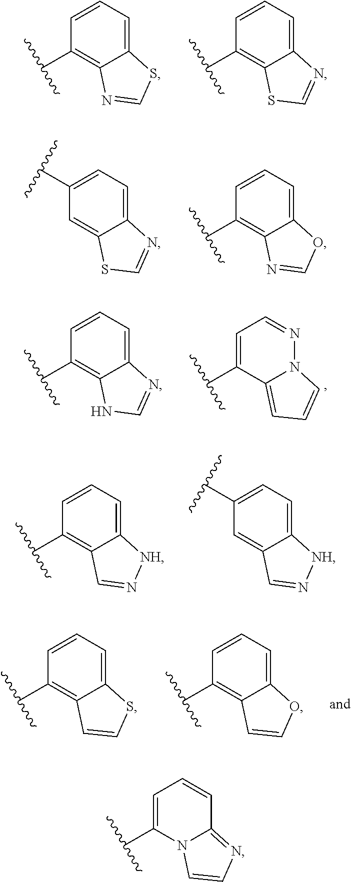

any of which is substituted with one or more R¹⁵.
B227. The compound of any one of embodiments B224-B226, wherein R⁶ is selected from:

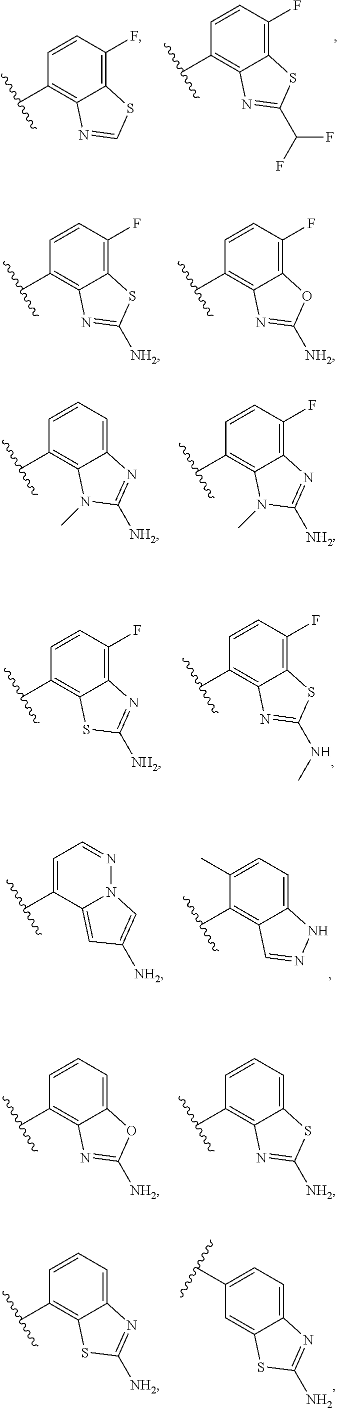

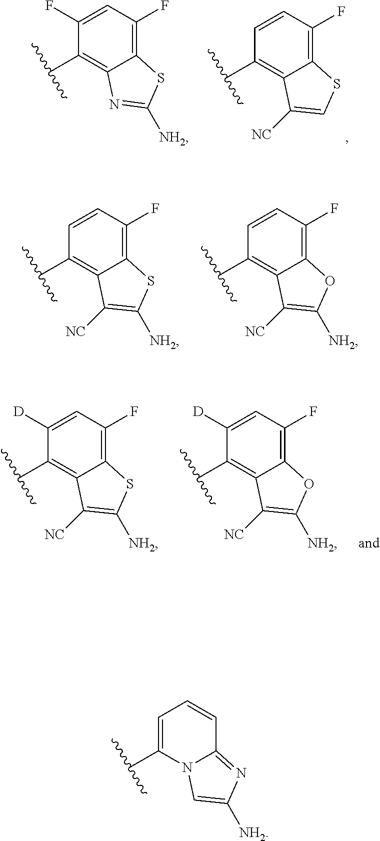

B228. The compound of any one of embodiments B224-B227, wherein R⁶ is selected from:

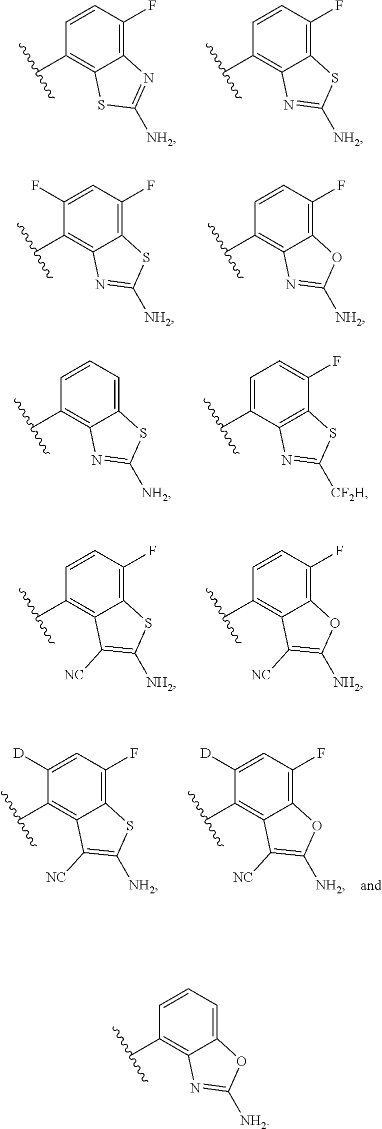

B229. The compound of any one of embodiments B95-B223, wherein R⁵ is a monocyclic heteroaryl that is substituted with one or more R¹⁵.
B230. The compound of embodiment B229, wherein R¹ is a pyridine substituted with one or more R¹³.
B231. The compound of embodiment B230, wherein R⁶ has the structure

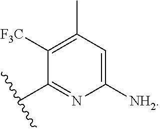 [SMILES: *c1nc(N)cc(C)c1C(F)(F)F]

B232. The compound of any one of embodiments B95-B231, wherein R¹ is selected from —OR⁸.
B233. The compound of embodiment B232, wherein R¹ is selected from:

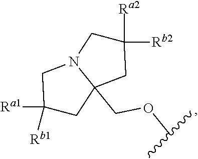

wherein R^{a1}, R^{a2}, R^{b1}, and R^{b2} are each independently selected from deuterium, halogen, C₁₋₆alkyl, —OR¹², and H, wherein R^{a2} and R^{b2} can optionally join together to form a 3-6 membered carbocycle, and wherein any C₁₋₆alkyl or 3-6 membered carbocycle is unsubstituted or is substituted with one or more R¹³.
B234. The compound of embodiment B233, wherein R¹ is selected from:

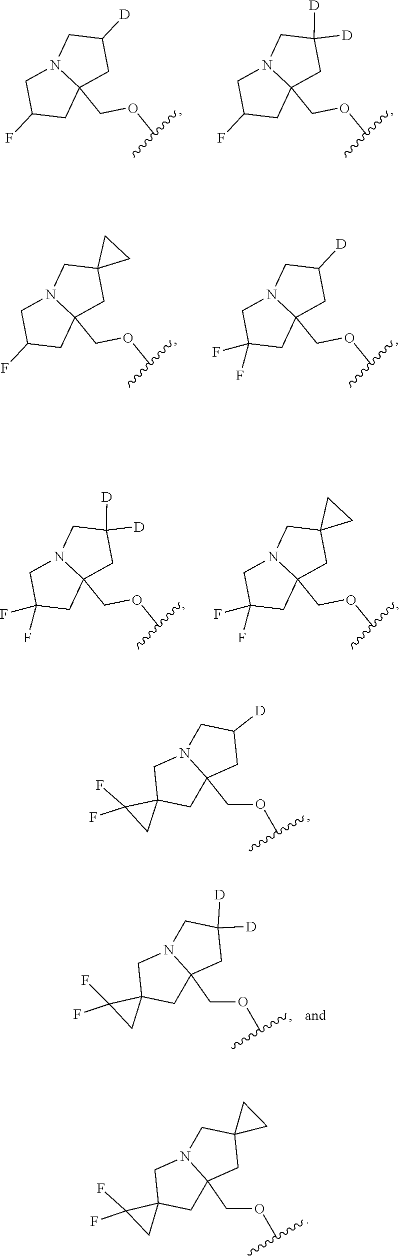

B235. The compound of embodiment B232, wherein R¹ is selected from:

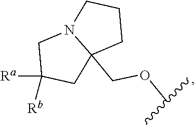

wherein R^{a} and R^{b} are each independently selected from halogen, C₁₋₆alkyl, —OR¹², and H, wherein any C₁₋₆alkyl is unsubstituted or is substituted with one or more R¹³.
B236. The compound of embodiment B235, wherein R¹ is selected from:

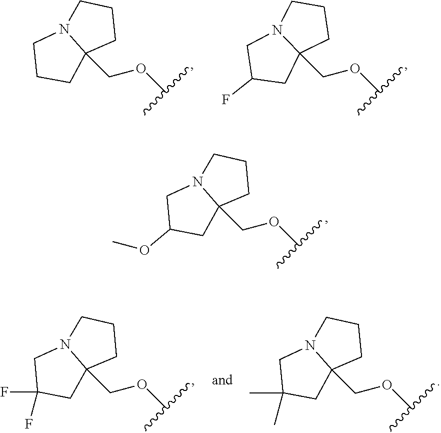 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

B237. The compound of embodiment B232, wherein R¹ is selected from:

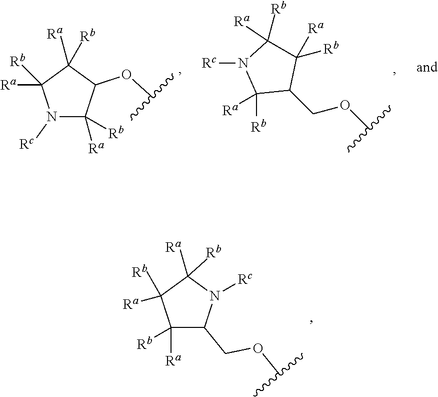

wherein each R^{a} and R^{b} is independently selected from halogen, C₁₋₆ alkyl, —OR¹², and H; and R^{c} is selected from C₁₋₆ alkyl, wherein an R^{a} and R^{b} or R^{c} optionally join together to form a 3-6 membered carbocycle or heterocycle, and wherein any C₁₋₆ alkyl or 3-6 membered carbocycle or heterocycle is unsubstituted or is substituted with one or more R¹³.
B238. The compound of embodiment B237, wherein R¹ is selected from:

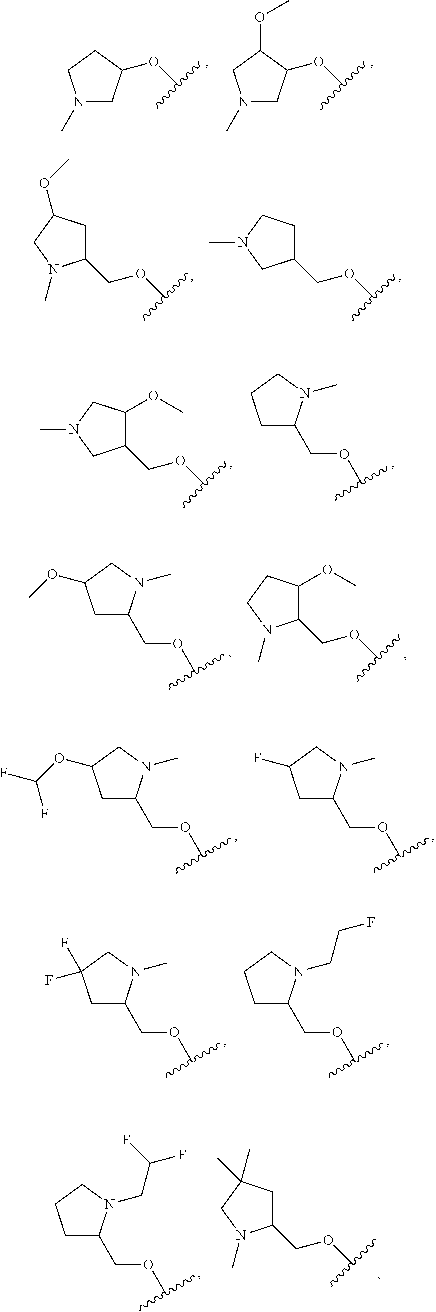

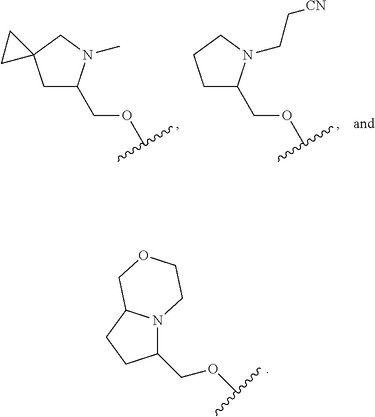

B239. The compound of any one of embodiments B95-B231, wherein R¹ is selected from:

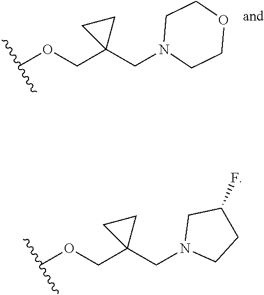 [SMILES: *OCC1(CN2CCOCC2)CC1 ; *OCC1(CN2CC[C@@H](F)C2)CC1]

B240. The compound of any one of embodiments B95-B239, wherein each R¹¹ is independently selected from —OR¹², ═O, —C(O)(Cᵢ-alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹², halogen, and C₁₋₆alkyl that is unsubstituted or substituted with one or more R².
B241. The compound of any one of embodiments B95-B240, wherein R³ is a halogen (e.g., F or Cl).
B242. The compound of any one of embodiments B95-B240, wherein R¹ is selected from C₁₋₆alkyl that is unsubstituted or substituted with one or more R¹³.
B243. The compound of embodiment B242, wherein R⁵ is selected from C₁₋₆alkyl that is substituted with one or more halogens or —CN.
B244. The compound of embodiment B243, wherein R⁵ is selected from —CF₂H, —CF, —CH₂CN, and —CH₂CH₃.
B245. The compound of embodiment B244, wherein R is —CF₃.
B246. The compound of any one of embodiments B95-B245, wherein the compound is a not a compound included in Table 2.
B247. A compound represented by Formula I:

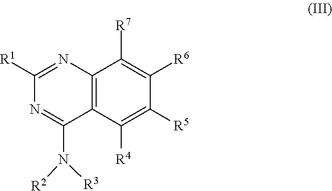

or a salt (e.g., pharmaceutically acceptable salt) thereof, wherein:
R¹ is selected from —OR⁸ and

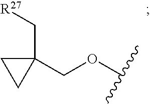

R² is selected from H and C₁₋₆alkyl, wherein any C₁₋₆ alkyl is unsubstituted or is substituted with one or more R¹³;
R³ is selected from a 3-11 membered carbocycle that is unsubstituted or substituted with one or more R¹⁰;
R⁴ is H;
R³ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁶ is a bicyclic heteroaryl substituted with one or more R¹⁵;
R⁷ is selected from halogen, —CN, and H;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹⁰ is independently selected from —OR¹², —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H,
each R¹⁵ is independently selected from deuterium, halogen, —N(R¹⁴)₂, —CN, and C₁₋₆alkyl, wherein any C₁₋₆alkyl is unsubstituted or substituted with one or more R¹³;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen; and
R^{a} and R^{b} are each independently selected from halogen, C₁₋₆ alkyl, —OR¹², and H, wherein any C₁₋₆alkyl is unsubstituted or is substituted with one or more R¹³, and wherein an R^{a} and R^{b} optionally join together to form a 3-6 membered carbocycle or heterocycle.
B248. The compound of embodiment B247, wherein R³ is a 3-6 membered carbocycle that is unsubstituted or is substituted with one or more R²⁰.
B249. The compound of embodiment B248, wherein R³ is a 3-6 membered carbocycle that is substituted with 1-4 R¹⁰.
B250. The compound of embodiment B248, wherein R³ is a cyclopropane that is substituted with 0-4 R¹⁰.
B251. The compound of embodiment B248, wherein R³ is a cyclobutane that is substituted with 0-4 R¹⁰.
B252. The compound of embodiment B248, wherein R³ is a cyclopentane that is substituted with 0-4 R¹⁰.
B253. The compound of embodiment B248, wherein R³ is a cyclohexane that is substituted with 0-4 R¹⁰.
B254. The compound of any one of embodiments B247-B249, wherein R³ is a spirocycle that is unsubstituted or is substituted with one or more R²⁰.
B255. The compound of any one of embodiments B247-B249, wherein R³ is a bridged carbocycle that is unsubstituted or is substituted with one or more R²⁰.
B256. The compound of any one of embodiments B247-B255, wherein R³ is substituted with one or more R¹⁰, wherein at least one R¹⁰ is selected from —OR¹² and a C₁₋₆alkyl substituted with —OH.
B257. The compound of any one of embodiments B247-B256, wherein R³ is substituted with one or more R¹⁰, wherein at least one R¹⁰ is an unsubstituted C₁₋₆alkyl.
B258. The compound of any one of embodiments B247-B257, wherein the compound is a compound according to Formula IA, IB, IC, ID, IE, or IF:

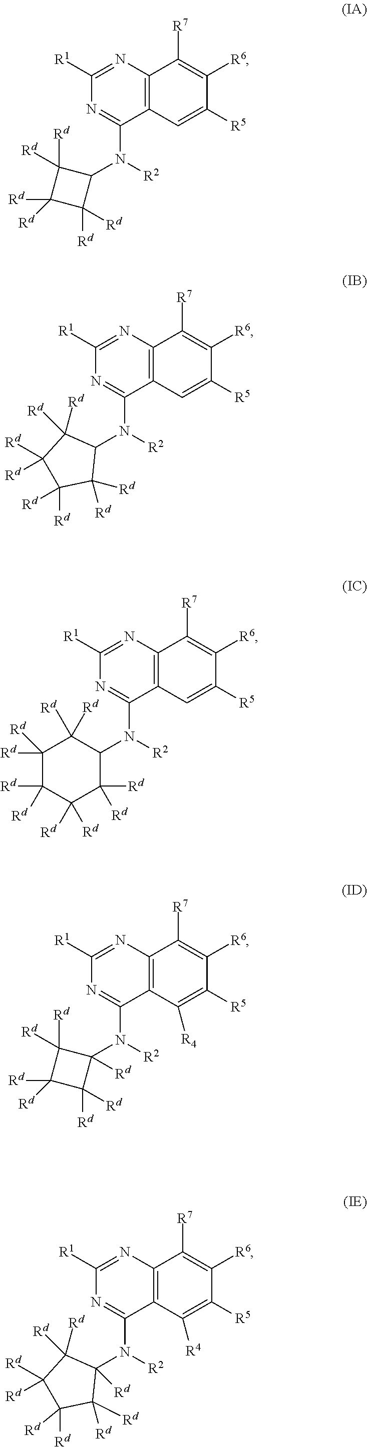

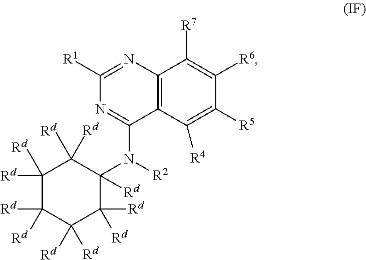

or a salt (e.g., pharmaceutically acceptable salt) thereof, wherein:
each R^{d} is independently selected from H, —OR¹², —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R².
B259. The compound of embodiment B258, wherein the compound is a compound according to Formula IA, or a salt (e.g., pharmaceutically acceptable salt) thereof.
B260. The compound of embodiment B258, wherein the compound is a compound according to Formula IB, or a salt (e.g., pharmaceutically acceptable salt) thereof.
B261. The compound of embodiment B258, wherein the compound is a compound according to Formula IC, or a salt (e.g., pharmaceutically acceptable salt) thereof.
B262. The compound of embodiment B258, wherein the compound is a compound according to Formula ID, or a salt (e.g., pharmaceutically acceptable salt) thereof.
B263. The compound of embodiment B258, wherein the compound is a compound according to Formula IE, or a salt (e.g., pharmaceutically acceptable salt) thereof.
B264. The compound of embodiment B258, wherein the compound is a compound according to Formula IF, or a salt (e.g., pharmaceutically acceptable salt) thereof.
B265. The compound of any one of embodiments B258-B264, wherein at least one R^{d} is selected from —OR¹², —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰.
B266. The compound of embodiment B265, wherein at least one R is selected from —OR¹² and C₁₋₆alkyl, wherein any C₁₋₆alkyl is unsubstituted or substituted with one or more R²⁰.
B267. The compound of any one of embodiments B247-B266, wherein R¹ is selected from:

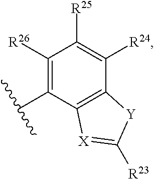

wherein:
   X is selected from N and C—CN;
   Y is selected from O and S;
   R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
   R²⁴, R²⁵, and R²⁶ are independently selected from H, deuterium, halogen, and C₁₋₆alkyl, wherein any C₁₋₆alkyl is unsubstituted or substituted with one or more R¹³.
B268. The compound of any one of embodiments B247-B267, wherein R⁶ is selected from:

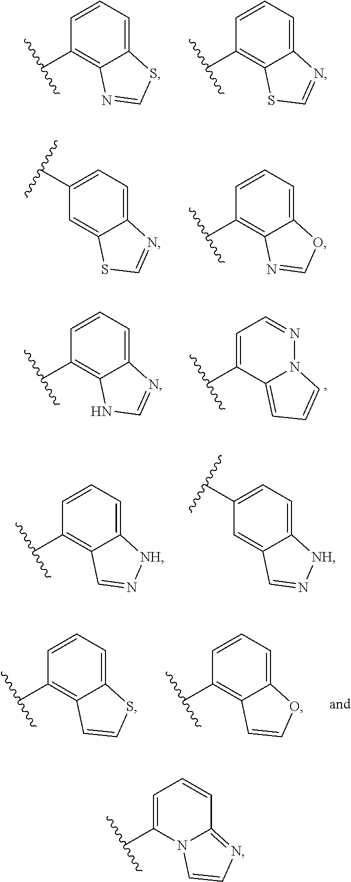

any of which is substituted with one or more R¹⁵.
B269. The compound of any one of embodiments B247-B268, wherein R⁶ is selected from:

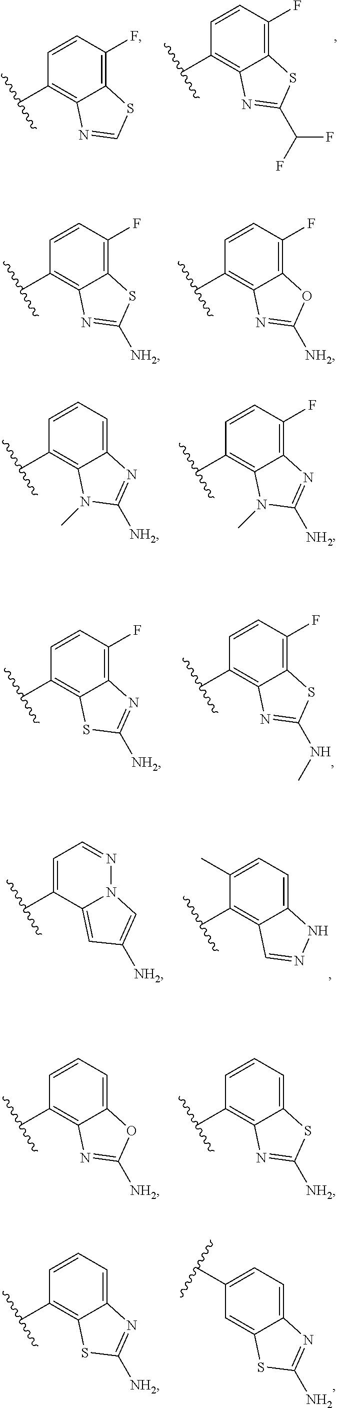

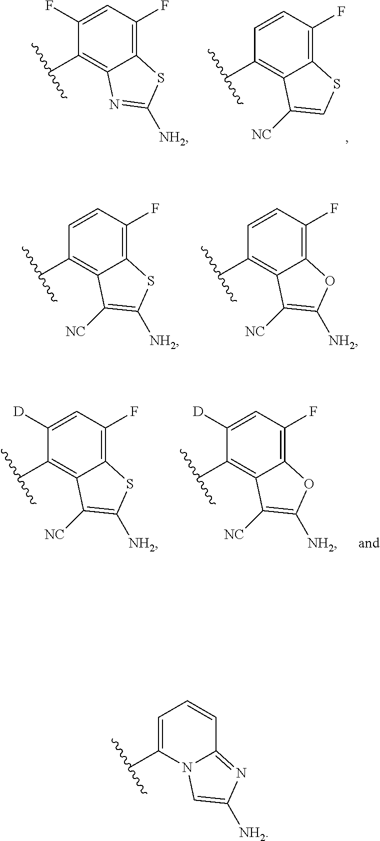

B270. The compound of any one of embodiments B247-B269, wherein R⁶ is selected from:

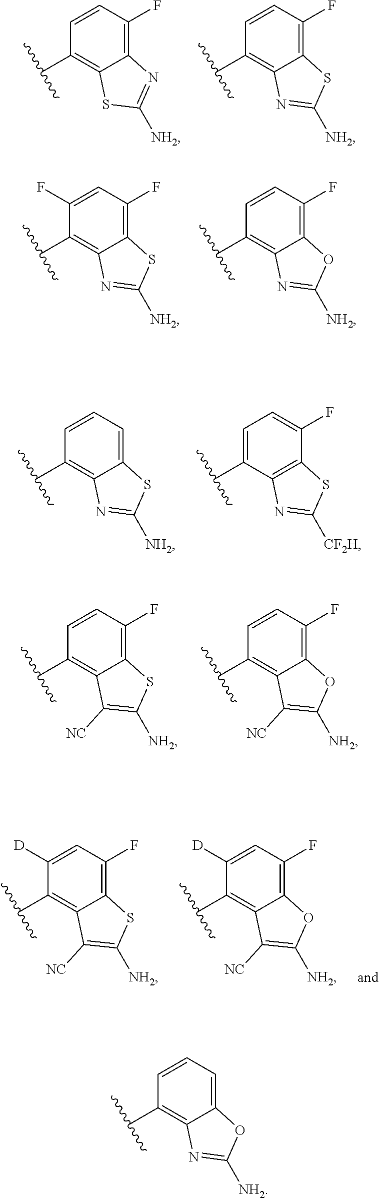

B271. The compound of any one of embodiments B247-B270, wherein the compound is a compound according to Formula IA1, IB1, IC1, ID1, IE1, or IF1:

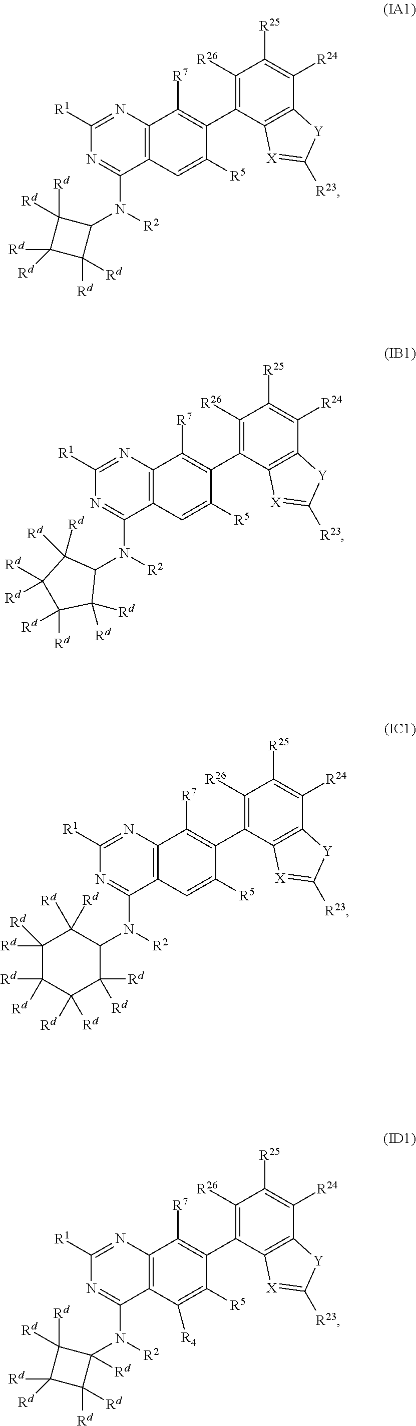

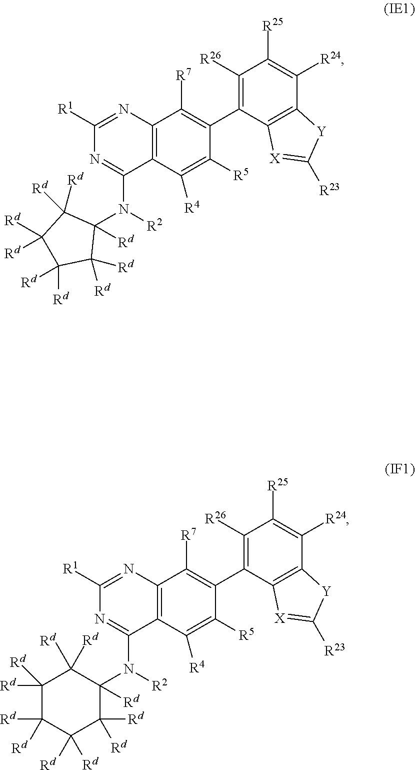

or a salt (e.g., pharmaceutically acceptable salt) thereof, wherein:
each R^{d} is independently selected from H, —OR¹², —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰;
X is selected from N and C—CN;
Y is selected from O and S;
R²³ is selected from —N(R¹²)₂, C₁₋₆alkyl, and C₁₋₆alkyl-N(R¹⁰)₂, wherein any C₁₋₆alkyl is unsubstituted or substituted with one or more R¹³, and
R²⁴, R²⁵, and R²⁶ are independently selected from H, deuterium, halogen, and C₁₋₆alkyl, wherein any C₁₋₆alkyl is unsubstituted or substituted with one or more R¹³.
B272. The compound of embodiment B271, wherein the compound is a compound according to Formula IA1, or a salt (e.g., pharmaceutically acceptable salt) thereof.
B273. The compound of embodiment B271, wherein the compound is a compound according to Formula IB1, or a salt (e.g., pharmaceutically acceptable salt) thereof.
B274. The compound of embodiment B271, wherein the compound is a compound according to Formula IC1, or a salt (e.g., pharmaceutically acceptable salt) thereof.
B275. The compound of embodiment B271, wherein the compound is a compound according to Formula ID1, or a salt (e.g., pharmaceutically acceptable salt) thereof.
B276. The compound of embodiment B271, wherein the compound is a compound according to Formula IE1, or a salt (e.g., pharmaceutically acceptable salt) thereof.
B277. The compound of embodiment B271, wherein the compound is a compound according to Formula IF1, or a salt (e.g., pharmaceutically acceptable salt) thereof.
B278. The compound of any one of embodiments B271-B277, wherein X is C—CN, Y is S, and R²³ is —N(R¹²)₂.
B279. The compound of any one of embodiments B271-B278, wherein one or more of R²⁴, R²⁵, and R²⁶ is a halogen (e.g., F).
B280. The compound of any one of embodiments B247-B279, wherein R¹ is selected from —OR⁸.
B281. The compound of embodiment B280, wherein R¹ is selected from:

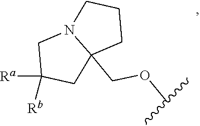

wherein R^{a} and R^{b} are each independently selected from halogen, C₁₋₆alkyl, —OR¹², and H, wherein any C₁₋₆alkyl is unsubstituted or is substituted with one or more R¹³.
B282. The compound of embodiment B281, wherein R¹ is selected from:

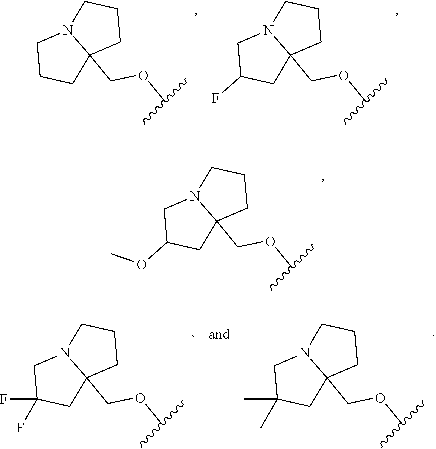 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

B283. The compound of embodiment B280, wherein R¹ is selected from:

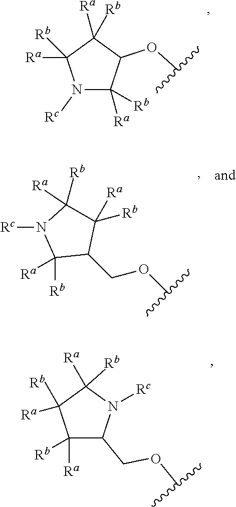

wherein each R^{a} and R^{b} is independently selected from halogen, C₁₋₆ alkyl, —OR¹², and H; and R is selected from C₁₋₆ alkyl, wherein any C₁₋₆ alkyl is unsubstituted or is substituted with one or more R¹³, and wherein an R^{a} and R^{b} or R^{c} optionally join together to form a 3-6 membered carbocycle or heterocycle.
B284. The compound of embodiment B283, wherein R¹ is selected from:

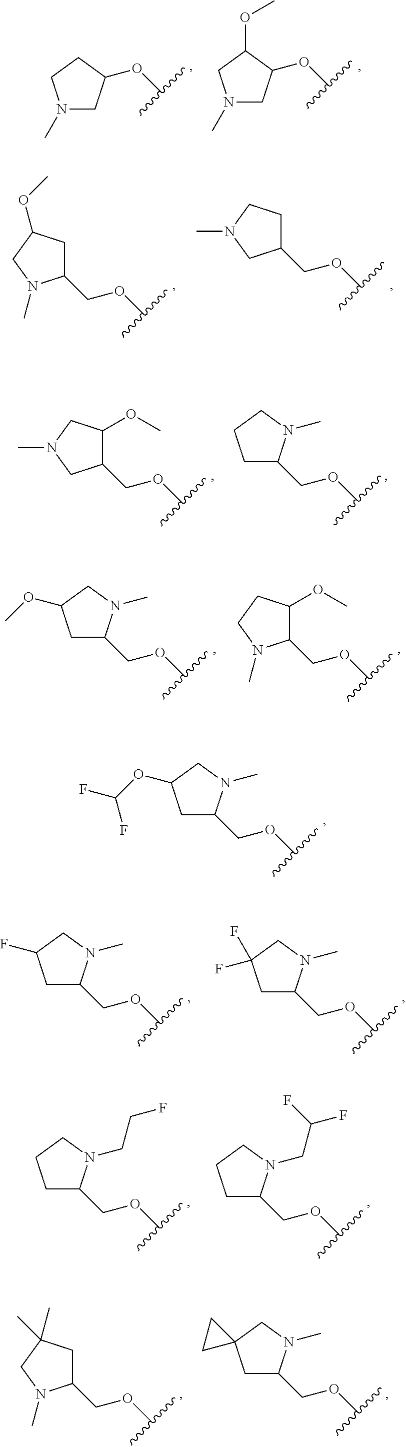

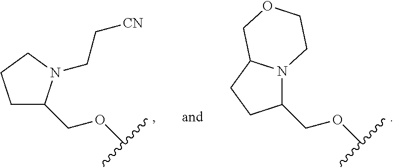 [SMILES: *OCC1CCCN1CCC#N ; *OCC1CCC2COCCN12]

B285. The compound of any one of embodiments B247-B279, wherein R¹ is selected from

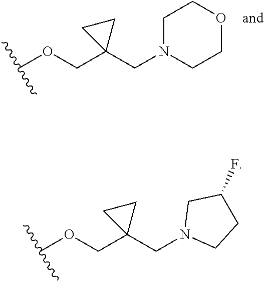 [SMILES: *OCC1(CN2CCOCC2)CC1 ; *OCC1(CN2CC[C@@H](F)C2)CC1]

B286. The compound of any one of embodiments B247-B285, wherein the compound is a compound according to Formula IA2, IB2, IC2, ID2, IE2, or IF2:

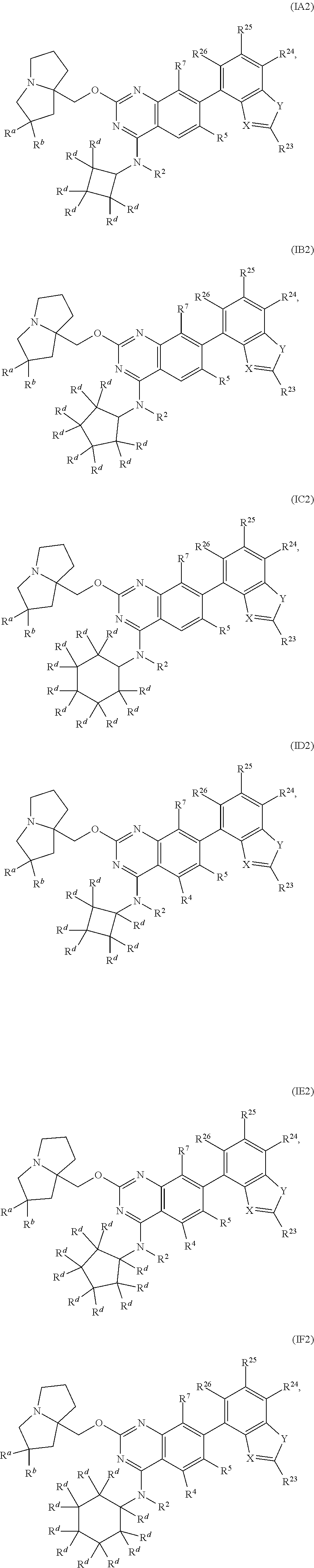

or a salt (e.g., pharmaceutically acceptable salt) thereof, wherein:
R^{a} and R^{b} are each independently selected from halogen, C₁₋₆ alkyl, —OR¹², and H, wherein any C₁₋₆alkyl is unsubstituted or is substituted with one or more R¹³;
each R^{d} is independently selected from H, —OR¹², —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, deuterium, halogen, and C₁₋₆akyl, wherein any C₁₋₆alkyl is unsubstituted or substituted with one or more R¹³.
B287. The compound of embodiment B286, wherein the compound is a compound according to Formula IA2, or a salt (e.g., pharmaceutically acceptable salt) thereof.
B288. The compound of embodiment B286, wherein the compound is a compound according to Formula IB2, or a salt (e.g., pharmaceutically acceptable salt) thereof.
B289. The compound of embodiment B286, wherein the compound is a compound according to Formula IC2, or a salt (e.g., pharmaceutically acceptable salt) thereof.
B290. The compound of embodiment B286, wherein the compound is a compound according to Formula ID2, or a salt (e.g., pharmaceutically acceptable salt) thereof.
B291. The compound of embodiment B286, wherein the compound is a compound according to Formula IE2, or a salt (e.g., pharmaceutically acceptable salt) thereof.
B292. The compound of embodiment B286, wherein the compound is a compound according to Formula IF2, or a salt (e.g., pharmaceutically acceptable salt) thereof.
B293. The compound of any one of embodiments B286-B292, wherein R¹ is a halogen (e.g., F).
B294. The compound of any one of embodiments B286-B293, wherein R^{b} is H.
B295. The compound of any one of embodiments B286-B294, wherein X is C—CN, Y is S, and R¹¹ is —N(R¹²)₂.
B296. The compound of any one of embodiments B286-B295, wherein one or more of R², R¹¹, and R^{D} is a halogen (e.g., F).
B297. The compound of any one of embodiments B247-B296, wherein R² is H.
B298. The compound of any one of embodiments B247-B296, wherein R² is selected from C₁₋₆alkyl that is unsubstituted or is substituted with one or more R³
B299. The compound of embodiment B298, wherein R² is selected from C₁₋₂alkyl.
B300. The compound of any one of embodiments B247-B299, wherein R⁵ is H.
B301. The compound of any one of embodiments B247-B299, wherein R is a halogen (e.g., F or Cl).
B302. The compound of any one of embodiments B247-B299, wherein R⁵ is selected from C₁₋₆alkyl that is unsubstituted or substituted with one or more R¹³.
B303. The compound of embodiment B302, wherein R¹ is selected from C₁₋₆alkyl that is substituted with one or more halogens or —CN.
B304. The compound of embodiment B303, wherein R is selected from —CF₂H, —CF₃, —CH₂CN, and —CH₂CH₃.
B305. The compound of embodiment B304, wherein R¹ is —CF₃.
B306. The compound of any one of embodiments B247-B305, wherein R⁷ is H.
B307. The compound of any one of embodiments B247-B305, wherein R⁷ is a halogen (e.g., F or Cl).
B308. The compound of any one of embodiments B247-B305, wherein R⁷ is —CN.
B309. A compound represented by Formula IV:

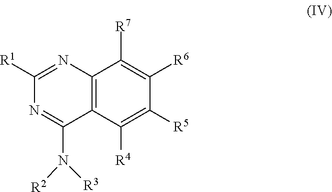

or a salt (e.g., pharmaceutically acceptable salt) thereof, wherein:
R′ is selected from —OR⁸ and

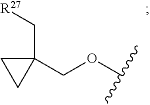

R² is selected from H and C₁₋₆alkyl, wherein any C₁₋₆ alkyl is unsubstituted or is substituted with one or more R¹³;
R³ is selected from C₁₋₆alkyl that is unsubstituted or is substituted with one or more R¹⁰;
R⁴ is H;
R⁵ is selected from H, halogen, —CN, —OR², a 3-6 membered heterocycle, a 5-6 membered heteroaryl, and C₁₋₆alkyl, wherein any C₁₋₆alkyl is unsubstituted or substituted with one or more R¹³;
R⁶ is a bicyclic heteroaryl substituted with one or more R⁵;
R⁷ is selected from halogen, —CN, and H;
R⁸ is selected from a heterocycle and an alkylheterocycle, wherein any heterocycle comprises 4-8 members and is unsubstituted or is substituted with one or more RV or R, and wherein an alkyl moiety of any alkylheterocycle is selected from C₁₋₆alkyl;
each R¹⁰ is independently selected from —OR¹⁴, ═O, —CN, —N(R¹⁴)₂, a 3-6 membered carbocycle, a 5-6 membered heteroaryl, —S(O)₂(C₁₋₆alkyl), —N(R¹⁴)C(O)(C₁₋₆alkyl), C₁₋₆alkyl, and halogen, wherein any C₁₋₆alkyl is unsubstituted or substituted with one or more R²⁰, any 3-6 membered carbocycle is unsubstituted or substituted with one or more R¹², and any 5-6 membered heteroaryl is unsubstituted or substituted with one or more R¹³;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹³;
each R²⁰ is independently selected from —OH, —OC₁₋₆alkyl, —CN, —NH₂. —NHC₁₋₆alkyl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen; and
R^{a} and R^{b} are each independently selected from halogen, C₁₋₆ alkyl, —OR¹², and H, wherein any C₁₋₆alkyl is unsubstituted or is substituted with one or more R¹³, and wherein an R^{a} and R^{b} optionally join together to form a 3-6 membered carbocyclic or heterocycle.
B310. Ile compound of embodiment B309, wherein the compound is a compound according to Formula IVA:

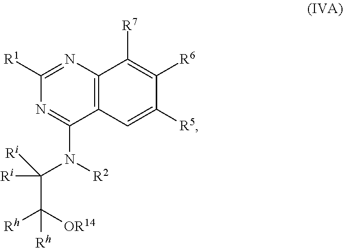

or a salt (e.g., pharmaceutically acceptable salt) thereof, wherein:
each Rⁱ and R^{h} are independently selected from H and R¹⁰.
B311. The compound of embodiment B310, wherein each Rⁱ and R^{h} are independently selected from H and C₁₋₆alkyl, wherein any C₁₋₆alkyl is unsubstituted or substituted with one or more R²⁰.
B312. The compound of embodiment B310 or B311, wherein R¹⁴ is H.
B313. The compound of any one of embodiments B309-B312, wherein R⁶ is selected from:

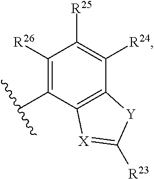

wherein:
   X is selected from N and C—CN;
   Y is selected from O and S;
   R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
   R²⁴, R²⁵, and R²⁶ are independently selected from H, deuterium, halogen, and C₁₋₆akyl, wherein any C₁₋₆alkyl is unsubstituted or substituted with one or more R¹³.
B314. The compound of any one of embodiments B309-B313, wherein R⁶ is selected from:

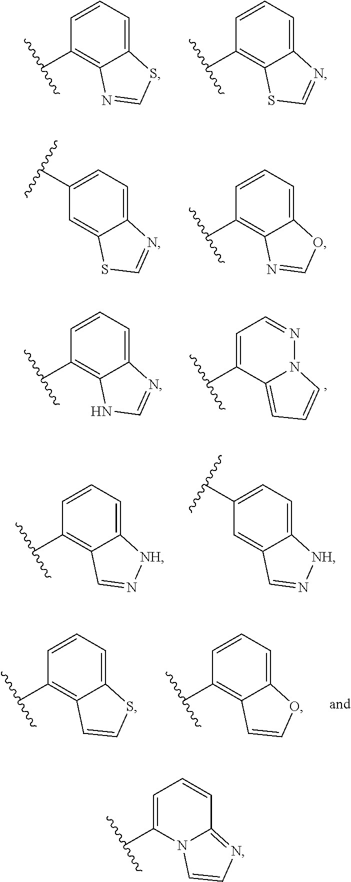

any of which is substituted with one or more R¹⁵.
B315. The compound of any one of embodiments B309-B314, wherein R⁶ is selected from:

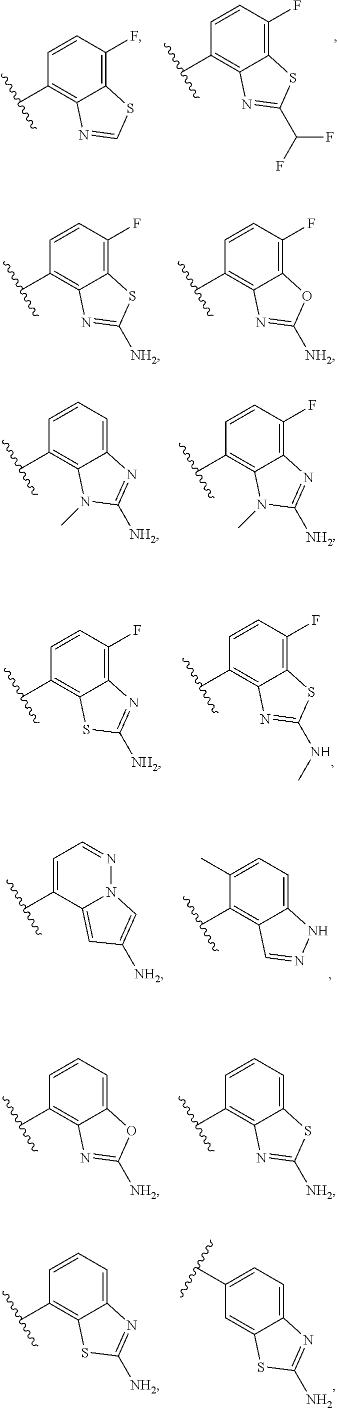

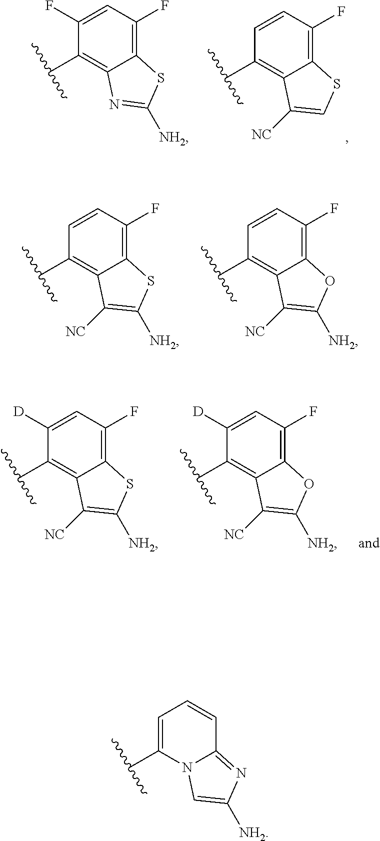

B315. The compound of any one of embodiments B309-B315, wherein R⁶ is selected from:

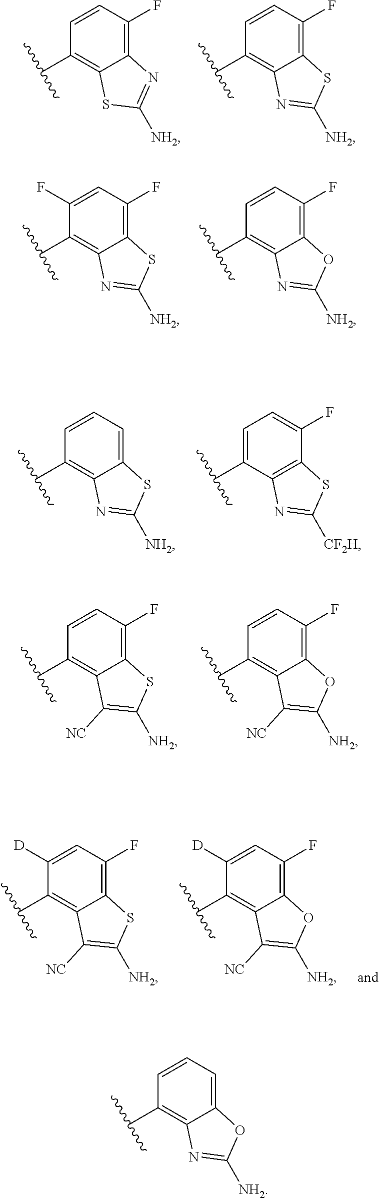

B317. The compound of embodiment B309, wherein the compound is a compound according to Formula IVB:

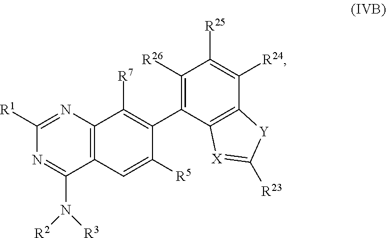

or a salt (e.g., pharmaceutically acceptable salt) thereof, wherein:
X is selected from N and C—CN;
Y is selected from O and S;
R¹¹ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, deuterium, halogen, —OR¹², and C₁₋₆alkyl, wherein any C₁₋₆alkyl is unsubstituted or substituted with one or more R¹³.
B318. The compound of any one of embodiments B309-B317, wherein R¹ is selected from —OR⁸.

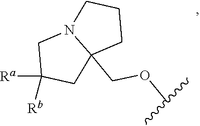

B319. The compound of embodiment B318, wherein R¹ is selected from: wherein R^{a} and R^{b} are each independently selected from halogen, C₁₋₆alkyl, —OR¹², and H, wherein any C₁₋₆alkyl is unsubstituted or is substituted with one or more R¹³.
B320. The compound of embodiment B321, wherein R¹ is selected from:

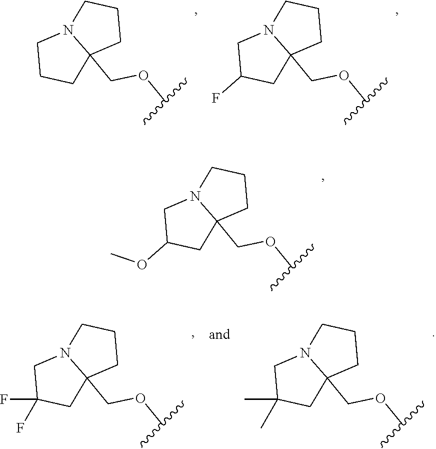 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

B321. The compound of embodiment B318, wherein R¹ is selected from:

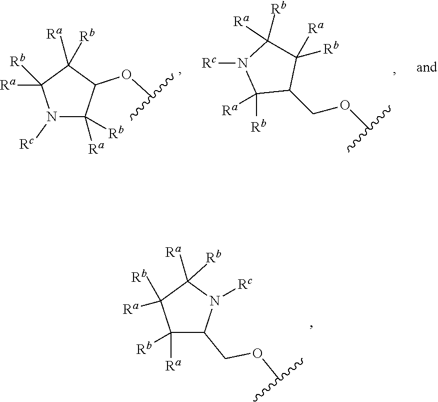

wherein each R^{a} and R^{b} is independently selected from halogen, C₁₋₆ alkyl, —OR², and H; and R is selected from C₁₋₆ alkyl, wherein any C₁₋₆ alkyl is unsubstituted or is substituted with one or more R¹¹, and wherein an R^{a} and R^{b} or R optionally join together to form a 3-6 membered carbocycle or heterocycle.
B322. The compound of embodiment B321, wherein R¹ is selected from:

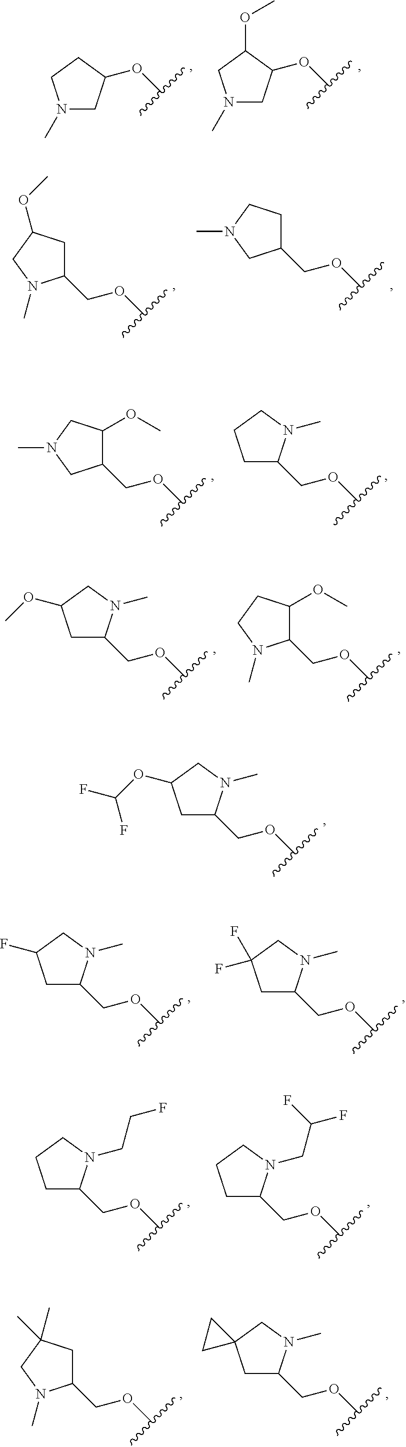

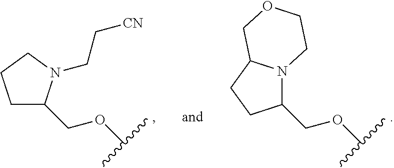 [SMILES: *OCC1CCCN1CCC#N ; *OCC1CCC2COCCN12]

B323. The compound of any one of embodiments B309-B317, wherein R¹ is selected from

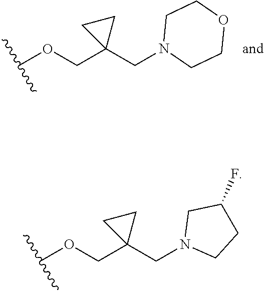 [SMILES: *OCC1(CN2CCOCC2)CC1 ; *OCC1(CN2CC[C@@H](F)C2)CC1]

B324. The compound of any one of embodiments B309-B323, wherein the compound is a compound according to Formula IVC:

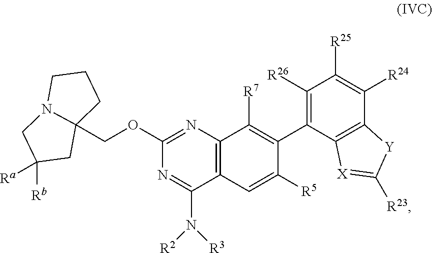

or a salt (e.g., pharmaceutically acceptable salt) thereof, wherein:
R^{a} and R^{b} are each independently selected from halogen, C₁₋₆ alkyl, —OR², and H, wherein any C₁₋₆alkyl is unsubstituted or is substituted with one or more R¹³;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, deuterium, halogen, —OR¹², and C₁₋₆alkyl, wherein any C₁₋₆alkyl is unsubstituted or substituted with one or more R¹³.
B325. The compound of embodiment B324, wherein R¹¹ is a halogen (e.g., F).
B326. The compound of embodiment B324 or B325, wherein R is H.
B327. The compound of any one of embodiments B317-B326, wherein X is C—CN, Y is S, and R²³ is selected from —N(R¹⁴)₂.
B328. The compound of any one of embodiments B317-B327, wherein at least one of R²⁴, R²⁵, and R²⁶ is a halogen (e.g., F).
B329. The compound of any one of embodiments B309 and B317-B328, wherein R³ is selected from C₁₋₆alkyl that is unsubstituted or is substituted with one or more R¹⁰.
B330. The compound of embodiment B329, wherein each R¹⁰ is independently selected from —OR¹², ═O, —CN, —NH(R¹⁵), —N(R¹⁵)₂, a 3-6 membered carbocycle, C₁₋₆alkyl, and halogen, wherein any C₁₋₆ alkyl is unsubstituted or substituted with one or more R²⁰, wherein any 3-6 membered carbocycle is unsubstituted or substituted with one or more R¹², and wherein each R¹⁶ is independently selected from C₁₋₆alkyl and C₂₋₆alkenyl.
B331. The compound of embodiment B330, wherein each R¹⁰ is independently selected from —OR¹², —CN, C₁₋₆alkyl, and halogen, wherein any C₁₋₆alkyl is unsubstituted or substituted with one or more R²⁰.
B332. The compound of embodiment B331, wherein each R¹⁰ is independently selected from halogen.
B333. The compound of embodiment B329, wherein R³ is selected from C₁₋₃alkyl that is unsubstituted.
B334. The compound of any one of embodiments B309 and B317-B333, wherein the moiety

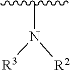

is selected from:

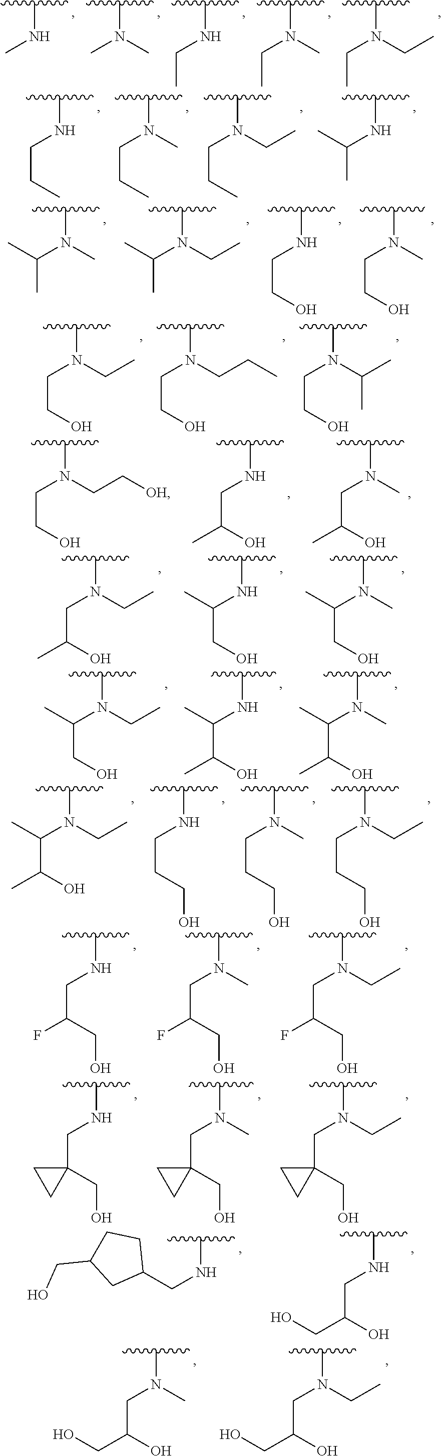

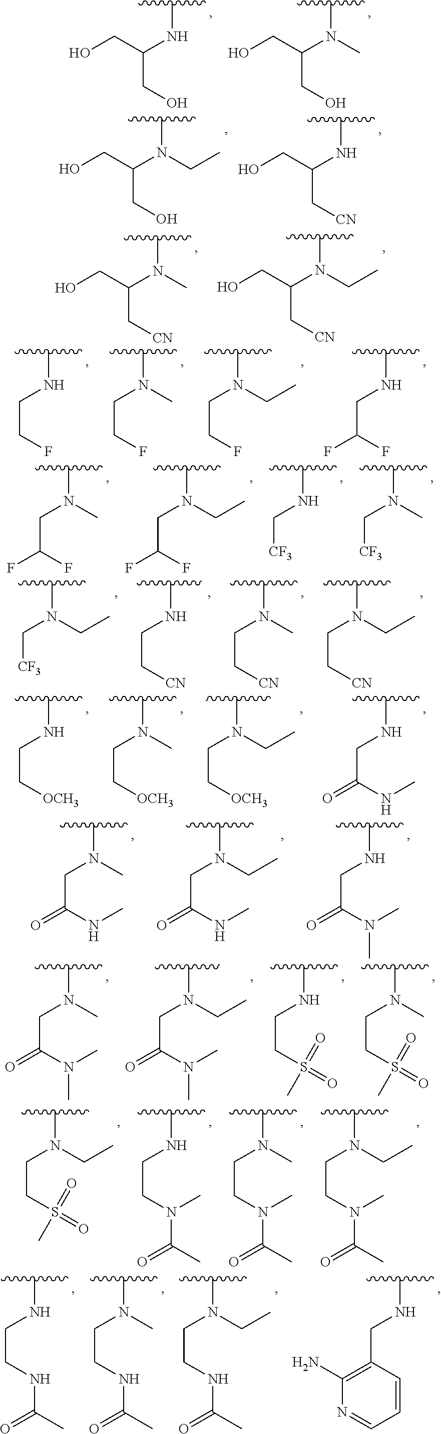

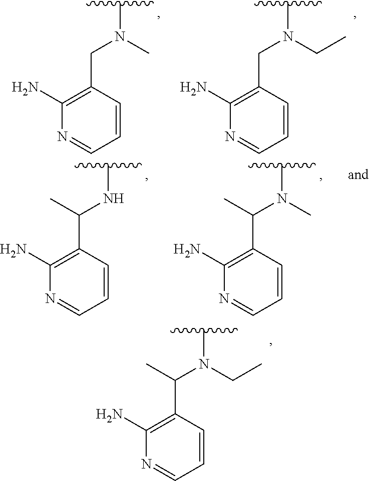

any of which is optionally further substituted with one or more R¹⁰.
B335. The compound of any one of embodiments B309-B334, wherein R² is H.
B336. The compound of any one of embodiments B309-B334, wherein R² is selected from C₁₋₆alkyl that is unsubstituted or is substituted with one or more R¹³.
B337. The compound of embodiment B336, wherein R² is selected from C₁₋₂alkyl that is unsubstituted.
B338. The compound of any one of embodiments B309-B337, wherein R⁵ is H.
B339. The compound of any one of embodiments B309-B337, wherein R⁵ is a halogen (e.g., F or Cl).
B340. The compound of any one of embodiments B309-B337, wherein R⁵ is selected from C₁₋₆alkyl that is unsubstituted or substituted with one or more R¹³.
B341. The compound of embodiment B340, wherein R⁵ is selected from C₁₋₆alkyl that is substituted with one or more halogens or —CN.
B342. The compound of embodiment B341, wherein R⁵ is selected from —CF₂H, —CF₃, —CH₂CN, and —CH₂CH₃.
B343. The compound of embodiment B342, wherein R⁵ is —CF₃.
B344. The compound of any one of embodiments B309-B343, wherein R¹ is H.
B345. The compound of any one of embodiments B309-B343, wherein R¹ is a halogen (e.g., F or Cl).
B346. The compound of any one of embodiments B309-B343, wherein R¹ is —CN.
B347. The compound of any one of embodiments B309-B346, wherein the compound is a not a compound included in Table 3
B348. A compound represented by Formula V:
   or a salt (e.g., pharmaceutically acceptable salt) thereof, wherein:

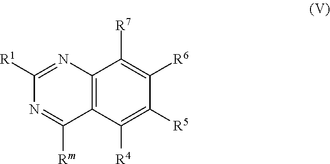

R¹ is selected from —OR⁸ and

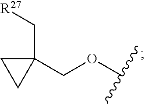

R^{m} is selected from hydrogen and C₁₋₆alkyl, wherein any C₁₋₆alkyl is unsubstituted or substituted with one or more R¹³;
R⁴ is H;
R⁵ is selected from halogen and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹⁴)₂, —CN, and C₁₋₆alkyl, wherein any C₁₋₆alkyl is unsubstituted or substituted with one or more R¹³;
R²⁷ is a 3-6 membered heterocycle including one or more heteroatoms selected from N, O, and S, wherein the heterocycle is unsubstituted or substituted with one or more R¹³;
each R²⁸ is independently selected from C₁₋₆alkyl and halogen; and
R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.
B349. The compound of embodiment B348, wherein R^{m} is hydrogen.
B350. The compound of embodiment B348 or B349, wherein the compound is a compound according to Formula VA:

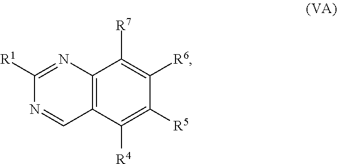

or a salt (e.g., pharmaceutically acceptable salt) thereof.
B351. The compound of embodiment B348, wherein the compound is a compound according to Formula VB:

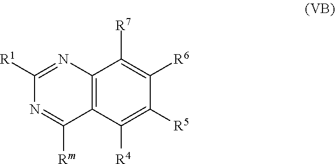

or a salt (e.g., pharmaceutically acceptably salt) thereof, wherein:
R^{m} is C₁₋₆alkyl that is unsubstituted or substituted with one or more R¹³.
B352. The compound of embodiment B348 or B351, wherein R^{m} is C₁₋₆alkyl that is unsubstituted or substituted with one or more R¹³.
B353. The compound of embodiment B352, wherein R^{m} is C₁₋₆alkyl that is unsubstituted.
B354. The compound of any one of embodiments B348-B353, wherein R⁶ is a bicyclic heteroaryl that is substituted with one or more R¹⁵.
B355. The compound of embodiment B354, wherein R⁶ is selected from:

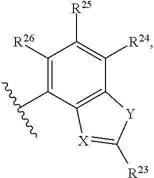

wherein X is selected from N and C—CN; Y is selected from O and S; R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and R²⁴, R²⁵, and R²⁶ are independently selected from H, deuterium, halogen, and C₁₋₆alkyl, wherein any C₁₋₆alkyl is unsubstituted or substituted with one or more R¹³.
B356. The compound of embodiment B355, wherein X is C—CN; Y is S; and R^{a} is —N(R¹²)₂.
B357. The compound of embodiment B355 or B356, wherein at least one of R²⁴, R²⁵, and R²⁶ is halogen.
B358. The compound of any one of embodiments B348-B353, wherein R⁶ is a monocyclic heteroaryl that is substituted with one or more R¹⁵.
B359. The compound of embodiment B358, wherein R⁶ is a pyridine substituted with one or more R¹⁵.
B360. The compound of embodiment B359, wherein R¹ has the structure

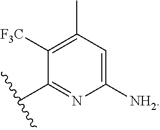 [SMILES: *c1nc(N)cc(C)c1C(F)(F)F]

B361. The compound of any one of embodiments B348-B360, wherein the compound is a compound according to Formula VC:

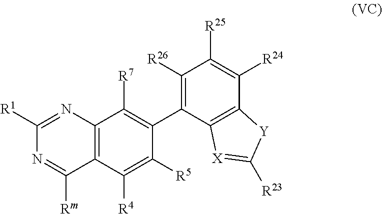

or a salt (e.g., pharmaceutically acceptable salt) thereof, wherein:
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, deuterium, halogen, —OR¹², and C₁₋₆alkyl, wherein any C₁₋₆alkyl is unsubstituted or substituted with one or more R¹³.
B362. The compound of embodiment B361, wherein X is C—CN, Y is S, and R²³ is selected from —N(R¹²)₂.
B363. The compound of embodiment B361 or B362, wherein at least one of R²⁴, R²⁵, and R²⁶ is halogen.
B364. The compound ofany one of embodiments B348-B363, wherein R¹ is selected from —OR⁸.
B365. The compound of embodiment B364, wherein R¹ is selected from:

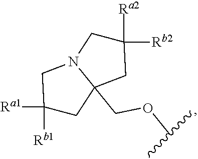

wherein R^{a1}, R^{a2}, R^{b1}, and R^{b2} are each independently selected from deuterium, halogen, C₁₋₆alkyl, —OR¹², and H, wherein R^{a2} and R^{b2} can optionally join together to form a 3-6 membered carbocycle, and wherein any C₁₋₆alkyl or 3-6 membered carbocycle is unsubstituted or is substituted with one or more R¹³.
B366. The compound of embodiment B365, wherein R¹ is selected from:

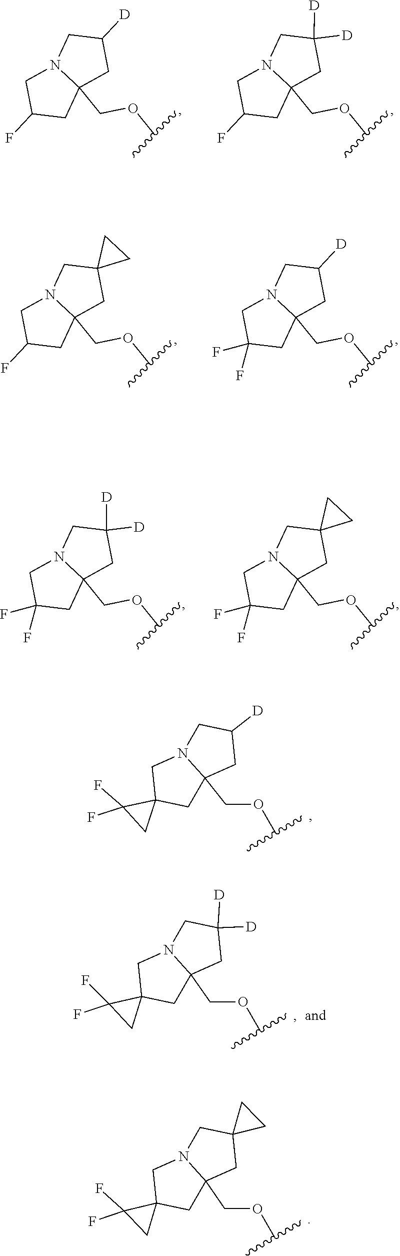

B367. The compound of embodiment B364, wherein R¹ is selected from:

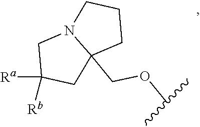

wherein R^{a} and R^{b} are each independently selected from halogen, C₁₋₆alkyl, —OR¹², and H, wherein any C₁₋₆alkyl is unsubstituted or is substituted with one or more R¹³.
B368. The compound of embodiment B367, wherein R¹ is selected from:

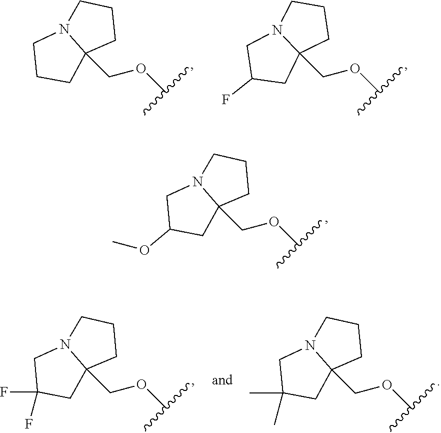 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

B369. The compound of embodiment B364, wherein R¹ is selected from:

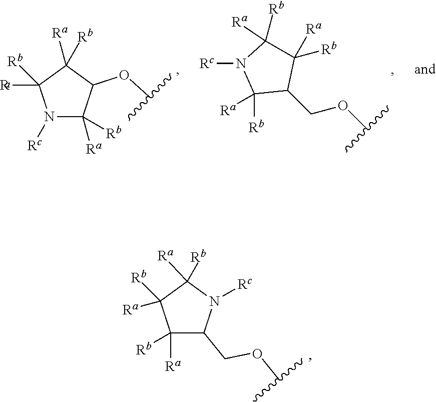

wherein each R^{a} and R^{b} is independently selected from halogen, C₁₋₆ alkyl, —OR¹², and H; and R^{c} is selected from C₁₋₆ alkyl, wherein an R^{a} and R^{b} or R^{c} optionally join together to form a 3-6 membered carbocycle or heterocycle, and wherein any C₁₋₆ alkyl or 3-6 membered carbocycle or heterocycle is unsubstituted or is substituted with one or more R¹³.
B370. The compound of embodiment B369, wherein R¹ is selected from:

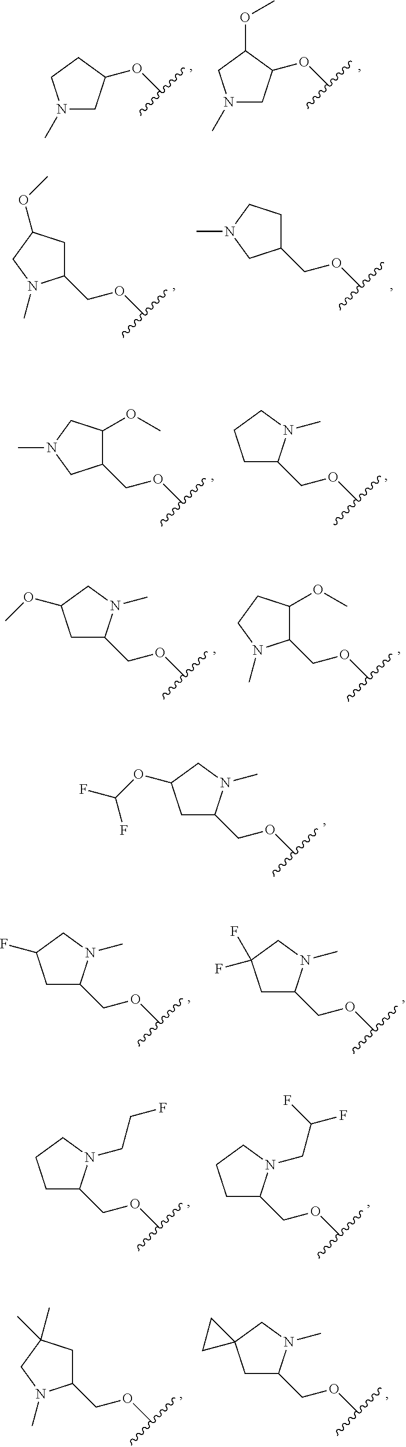

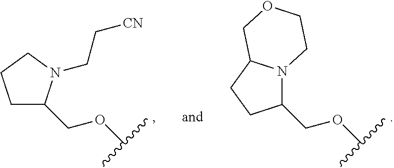 [SMILES: *OCC1CCCN1CCC#N ; *OCC1CCC2COCCN12]

B371. The compound of any one of embodiments B348-B363, wherein R¹ is selected from

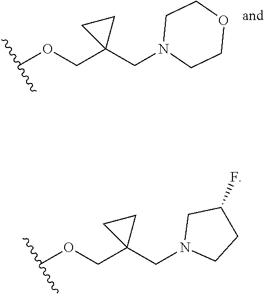 [SMILES: *OCC1(CN2CCOCC2)CC1 ; *OCC1(CN2CC[C@@H](F)C2)CC1]

B372. The compound of any one of embodiments B348-B371, wherein R⁵ is a halogen (e.g., F or Cl).
B373. The compound of any one of embodiments B348-B371, wherein R³ is selected from C₁₋₆alkyl that is unsubstituted or substituted with one or more R¹³.
B374. The compound of embodiment B373, wherein R⁵ is selected from C₁₋₆alkyl that is substituted with one or more halogens or —CN.
B375. The compound of embodiment B374, wherein R⁵ is selected from —CF₂H, —CF₃, —CH₂CN, and —CH₂CH₃.
B376. The compound of embodiment B375, wherein R⁵ is —CF₃.
B377. A compound shown in Table 5, or a salt (e.g., pharmaceutically acceptable salt) thereof.
B378. A compound shown in Table 6, or a salt (e.g., pharmaceutically acceptable salt) thereof
B379. A compound shown in Table 7, or a salt (e.g., pharmaceutically acceptable salt) thereof.
B380. A pharmaceutical composition comprising a compound of any one of embodiments B1-B379, or a salt (e.g., pharmaceutically acceptable salt) thereof, and a pharmaceutically acceptable excipient.
B381. A compound of any one of embodiments B1-B379, or a salt (e.g., pharmaceutically acceptable salt) thereof, for use as a medicament.
B382. The compound of embodiment B381, wherein the medicament is useful in the prevention or treatment of a disease, disorder, or condition ameliorated by the inhibition of KRAS having a Q61H, G13D, G12D, G12V, G12C, G12S, G12A, or G12R mutation, or wild-type KRAS.
B383. The compound of embodiment B382, wherein the medicament is useful in the prevention or treatment of a disease, disorder, or condition ameliorated by the inhibition of wild-type KRAS.
B384. The compound of embodiment B382, wherein the medicament is useful in the prevention or treatment of a disease, disorder, or condition ameliorated by the inhibition of KRAS having a G12D, G12R, or G12V mutation.
B385. The compound of any one of embodiments B381-B384, wherein the medicament is useful in the prevention or treatment of a cancer.
B386. The compound of embodiment B385, wherein the cancer is selected from the group consisting of pancreatic cancer, colorectal cancer, and lung cancer.
B387. A compound of any one of embodiments B1-B379, or a salt (e.g., pharmaceutically acceptable salt) thereof, for use in the treatment of a disease, disorder, or condition.
B388. The compound of embodiment B387, wherein the disease, disorder, or condition is a cancer.
B389. The compound of embodiment B388, wherein the cancer is selected from the group consisting of pancreatic cancer, colorectal cancer, and lung cancer.
B390. The compound of any one of embodiments B387-B389, wherein the compound is used in the treatment of a disease, disorder, or condition in a subject in need thereof.
B391. A compound of any one of embodiments B1-B379, or a salt (e.g., pharmaceutically acceptable salt) thereof, for use in the manufacture of a medicament.
B392. The compound of embodiment B391, wherein the medicament is useful in the prevention or treatment of a disease, disorder, or condition ameliorated by the inhibition of KRAS having a Q61H, G13D, G12D, G12V, G12C, G12S, G12A, or G12R mutation, or wild-type KRAS.
B393. The compound of embodiment B392, wherein the medicament is useful in the prevention or treatment of a disease, disorder, or condition ameliorated by the inhibition of wild-type KRAS.
B394. The compound of embodiment B392, wherein the medicament is useful in the prevention or treatment of a disease, disorder, or condition ameliorated by the inhibition of KRAS having a G12D, G12R, or G12V mutation.
B395. The compound of any one of embodiments B391-B394, wherein the medicament is useful in the treatment of a cancer.
B396. The compound of embodiment B395, wherein the cancer is selected from the group consisting of pancreatic cancer, colorectal cancer, and lung cancer.
B397. A method, comprising administering a therapeutically effective amount of a compound of any one of embodiments B1-B379, or a salt (e.g., pharmaceutically acceptable salt) thereof, to a subject in need thereof.
B398. The method of embodiment B397, wherein the subject has a disease, disorder, or condition ameliorated by the inhibition of KRAS having a Q61H, G3D, G12D, G12V, G12C, G12S, G12A, or G12R mutation, or wild-type KRAS.
B399. The method of embodiment B398, wherein the disease, disorder, or condition is ameliorated by the inhibition of wild-type KRAS.
B400. The method of embodiment B398, wherein the disease, disorder, or condition is ameliorated by the inhibition of KRAS having a G12D, G12R, or G12V mutation.
B401. The method of any one of embodiments B397-B400, wherein the subject has a cancer.
B402. The method of embodiment B401, wherein the subject was previously diagnosed with the cancer.
B403. The method of embodiment B401, wherein the subject has previously undergone a treatment regimen for the cancer.
B404. The method of embodiment B402 or B403, wherein the subject has previously entered remission from the cancer.
B405. The method of any one of embodiments B401-B404, wherein the cancer is selected from the group consisting of pancreatic cancer, colorectal cancer, and lung cancer.
B406. The method of any one of embodiments B397-B405, wherein the compound, or the salt thereof, is administered in combination with an additional therapeutic agent.
B407. The use of a compound of any one of embodiments B1-B379, or a salt (e.g., pharmaceutically acceptable salt) thereof, for the manufacture of a medicament for the treatment of a cancer.
B408. The use of embodiment B407, wherein the cancer is selected from the group consisting of pancreatic cancer, colorectal cancer, and lung cancer.
B409. A method, comprising contacting a KRAS protein with a compound of any one of embodiments B1-B379, or a salt (e.g., pharmaceutically acceptable salt) thereof.
B410. The method of embodiment B409, wherein contacting the KRAS protein with the compound modulates KRAS.
B411. The method of embodiment B409 or B410, wherein the KRAS protein has a Q61H, G13D, G12D, G12V, G12C, G12S, G12A, or G12R mutation.
B412. The method of embodiment B409 or B410, wherein the KRAS protein is a wild-type KRAS protein.
B413. The method of any one of embodiments B409-B412, wherein the KRAS protein is in an active (GTP-bound) state.
B414. The method of any one of embodiments B409-B412, wherein the KRAS protein is in an inactive (GDP-bound) state.
B415. The method of any one of embodiments B409-B414, wherein the KRAS protein is located within a cell.
B416. The method of embodiment B415, wherein the cell is located within a subject.
B417. The method of embodiment B416, wherein the subject is a human.
B418. The method of embodiment B416 or B417, wherein the subject has a cancer.
B419. The method of embodiment B418, wherein the cancer is selected from the group consisting of pancreatic cancer, colorectal cancer, and lung cancer.
B420. A method of inhibiting the function of a wild-type KRAS protein or a KRAS protein having a Q61H, G13D, G12D, G12V, G12C, G12S, G12A, or G12R mutation, comprising contacting the KRAS protein with a compound of any one of embodiments B1-B379, or a salt (e.g., pharmaceutically acceptable salt) thereof.
B421. The method of embodiment B420, wherein the KRAS protein is a wild-type KRAS protein.
B422. The method of embodiment B420, wherein the KRAS protein has a Q61H, G13D, G12D, G12V, G12C, G12S, G12A, or G12R mutation.
B423. The method of embodiment B422, wherein the KRAS protein has a G12D, G12V, or G12R mutation.
B424. The method of any one of embodiments B420-B423, wherein the KRAS protein is in an active (GTP-bound) state.
B425. The method of any one of embodiments B420-B423, wherein the KRAS protein is in an inactive (GDP-bound) state.
B426. The method of any one of embodiments B420-B425, wherein the KRAS protein is located within a cell.
B427. The method of embodiment B426, wherein the cell is located within a subject.
B428. The method of embodiment B427, wherein the subject is a human.
B429. The method of embodiment B427 or B428, wherein the subject has a cancer.
B430. The method of embodiment B429, wherein the cancer is selected from the group consisting of pancreatic cancer, colorectal cancer, and lung cancer.
B431. A compound capable of inhibiting a wild-type KRAS protein or a KRAS protein having a Q61H, G13D, G12D, G12V. G12C, G12S, G12A, or G12R mutation in both its active (GTP-bound) and inactive (GDP-bound) state.
B432. The compound of embodiment B431, wherein the compound:
   (i) has IC₅₀≤0.1 μM, 0.1 μM<IC₅₀1 μM, 1 μM<IC₅₀≤10 μM, or 10 μM<IC₅₀ in the assay of Biological Example 1 (e.g., a protein:protein interaction (PPI) Homogenous Time Resolved Fluorescence (HTRF) analysis of 50 nM Avi-KRAS G12D (amino acids 1-169) GppNHp/RAF1 RBD-3×FLAG (52-151); 50 nM Avi-KRAS G12R (amino acids 1-169) GppNHp/RAF1 RBD-3×FLAG (52-151); 50 nM Avi-KRAS G12V (amino acids 1-169) GppNHp/RAF1 RBD-3×FLAG (52-151); 50 nM Avi-KRAS WT (amino acids 1-169) GppNHp/RAF1 RBD-3×FLAG (52-151); and/or 75 nM Avi-RAF1 RBD-3×FLAG; and/or
   (ii) has IC₅₀ 0.1 μM, 0.1 μM<IC₅₀≤1 μM, or IC₅₀>1 μM in the assay of Biological Example 2 (e.g., cell-based pERK HTRF assay in GP2d (G12D) and SW620 (G12V) cell).
B433. The compound of embodiment B432, wherein the compound:
   (i) has IC₅₀≤0.1 μM or 0.1 μM<IC₅₀≤1 μM in the assay of Biological Example 1 (e.g., a protein:protein interaction (PPI) Homogenous Time Resolved Fluorescence (HTRF) analysis of 50 nM Avi-KRAS G12D (amino acids 1-169) GppNHp/RAF RBD-3×FLAG (52-151); 50 nM Avi-KRAS G12R (amino acids 1-169) GppNHp/RAF1 RBD-3×FLAG (52-151): 50 nM Avi-KRAS G12V (amino acids 1-169) GppNHp/RAF1 RBD-3×FLAG (52-151): 50 nM Avi-KRAS WT (amino acids 1-169) GppNHpi/RAF1 RBD-3×FLAG (52-151); and/or 75 nM Avi-RAF1 RBD-3×FLAG; and/or
   (ii) has IC₅₀≤0.1 μM or 0.1 μM<IC₅₀≤1 μM in the assay of Biological Example 2 (e.g., cell-based pERK HTRF assay in GP2d (G12D) and SW620 (G12V) cell).
B434. The compound of any one of embodiments B431-B433, wherein the compound is capable of irreversibly binding the KRAS protein.
B435. The compound of any one of embodiments B431-B433, wherein the compound is capable of reversibly binding the KRAS protein.
B436. The compound of any one of embodiments B431-B435, wherein the compound is a compound according to any one of embodiments B1-B379.

The following numbered embodiments, while non-limiting, are exemplary of certain aspects of the present disclosure:
C1. A compound represented by Formula II′:

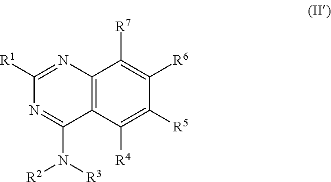

or a salt (e.g., pharmaceutically acceptable salt) thereof, wherein:
R¹ is selected from —OR⁸ and

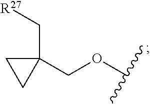

R² is selected from H, C₁₋₆alkyl, and a 3-6 membered carbocycle, wherein any C₁₋₆ alkyl is unsubstituted or is substituted with one or more R¹³;
R³ is selected from a 4-9 membered heterocycle that is unsubstituted or substituted with one or more R¹⁰;
R⁴ is H;
R⁵ is selected from H, halogen, —CN, —OR², a 3-6 membered heterocycle, a 5-6 membered heteroaryl, and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹⁰ is independently selected from —OR¹², —O, —C(O)(C₁₋₆ alkylene)CN, —C(O(C₁₋₆ alkylene)OH, —C(O)(C₁₋₆alkyl), —C(O)O(C₁₋₆alkyl), —C(O)N(R¹⁴)₂, —C(O)OR¹², —C(O)(3-6 membered carbocycle), —C(O)(3-7 membered heterocycle), —C(O)(5-6 membered heteroaryl), —C(O)O(3-6 membered heterocycle), —C(O)O(C₁₋₆ alkylene)(3-6 membered heterocycle), —S(O)₂(C₁₋₆alkyl), halogen, a 3-6 membered carbocycle, and C₁₋₆alkyl, wherein any C₁₋₆alkyl is unsubstituted or substituted with one or more R²⁰, wherein any 3-6 membered carbocycle, 5-6 membered heteroaryl, or 3-6 membered heterocycle is unsubstituted or substituted with one or more R¹² or R²⁰, and wherein two R¹⁰s optionally join together to form, together with the atom(s) to which they are attached, a 3-6 membered carbocycle or heterocycle;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from a 3-6 membered carbocycle, a 3-6 membered heterocycle, C₁₋₆ alkyl, C₂₋₆ alkenyl, and H, wherein any C₁₋₆ alkyl is optionally deuterated;
each R¹⁵ is independently selected from deuterium, halogen, —N(R¹⁵)₂, —CN, and C₁₋₆alkyl, wherein any C₁₋₆alkyl is unsubstituted or substituted with one or more R¹³;
each R^{a} is independently selected from —OH, —OC₁₋₆alkyl, ═O, —CN, —NH₂, —NHC₁₋₆alkyl, —C(O)(C₁₋₆alkyl), a 3-6 membered carbocycle, phenyl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen; and
R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.
C2. A compound represented by Formula II:

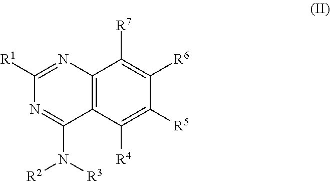

or a salt (e.g., pharmaceutically acceptable salt) thereof, wherein:
R¹ is selected from —OR⁸ and

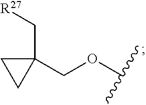

R² is selected from H, C₁₋₆alkyl, and a 3-6 membered carbocycle, wherein any C₁₋₄ alkyl is unsubstituted or is substituted with one or more R¹³;
R³ is selected from a 4-9 membered heterocycle that is unsubstituted or substituted with one or more R¹⁰, provided that (i) when the heterocycle contains a nitrogen atom, the nitrogen atom is substituted with R¹⁰, or (ii) the heterocycle does not comprise an —NH— moiety;
R⁴ is H;
R⁵ is selected from H, halogen, —CN, —OR², a 3-6 membered heterocycle, a 5-6 membered heteroaryl, and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹⁰ is independently selected from —OR¹², ═O, —C(O)C₁₋₆ alkylene)CN, —C(O)(C₁₋₆ alkylene)OH, —C(O)(C₁₋₆alkyl), —C(O)O(C₁₋₆alkyl), —C(O)N(R¹⁴)₂, —C(O)OR¹², —C(O)(3-6 membered carbocycle), —C(O)(3-7 membered heterocycle), —C(O)(5-6 membered heteroaryl), —C(O)O(3-6 membered heterocycle), —C(O)O(C₁₋₆ alkylene)(3-6 membered heterocycle), —S(O)₂(C₁₋₆alkyl), halogen, a 3-6 membered carbocycle, and C₁₋₆alkyl, wherein any C₁₋₆alkyl is unsubstituted or substituted with one or more R²⁰, wherein any 3-6 membered carbocycle, 5-6 membered heteroaryl, or 3-6 membered heterocycle is unsubstituted or substituted with one or more R¹² or R²⁰, and wherein two R¹⁰s optionally join together to form, together with the atom(s) to which they are attached, a 3-6 membered carbocycle or heterocycle;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from a 3-6 membered carbocycle, a 3-6 membered heterocycle, C₁₋₆ alkyl, C₂₋₆ alkenyl, and H, wherein any C₁₋₆ alkyl is optionally deuterated;
each R¹⁵ is independently selected from deuterium, halogen, —N(R¹⁴)₂, —CN, and C₁₋₆alkyl, wherein any C₁₋₆alkyl is unsubstituted or substituted with one or more R¹³;
each R²⁰ is independently selected from —OH, —OC₁₋₆alkyl, ═O, —CN, —NH₂, —NHC₁₋₆alkyl, —C(O)(C₁₋₆alkyl), a 3-6 membered carbocycle, phenyl, and halogen;
R²⁷ is a 3-6 membered heterocycle including one or more heteroatoms selected from N, O, and S, wherein the heterocycle is unsubstituted or substituted with one or more R²¹;
each R²⁸ is independently selected from C₁₋₆alkyl and halogen; and
R^{a} and R^{b} are each independently selected from deuterium, halogen, C₁₋₆ alkyl, a 3-6 membered carbocycle, —OR², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.
C3. The compound of embodiment C1 or C2, wherein R³ is a 4-6 membered heterocycle that includes 1 heteroatom selected from 0. S, and N.
C4. The compound of embodiment C3, wherein R³ is a 4-6 membered heterocycle that includes 1 heteroatom selected from O, S, and N, wherein the heterocycle is substituted with 1-4 R¹⁰, provided that when the heterocycle contains a nitrogen atom, the nitrogen atom is substituted with R¹⁰.
C5. The compound of any one of embodiments C1-C4, wherein R³ is a pyrrolidine that is substituted with 0-4 R¹⁰, provided that the nitrogen atom is substituted with R¹⁰.
C6. The compound of any one of embodiments C1-C4, wherein R³ is an oxetane or thietane that is substituted with 0-4 R¹⁰.
C7. The compound of any one of embodiments C1-C4, wherein R¹ is a tetrahydrofuran or tetrahydrothiopene that is substituted with 0-4 R¹⁰.
C8. The compound of any one of embodiments C1-C4, wherein R³ is a 4-6 membered heterocycle that is substituted with one or more R¹⁰, wherein at least one R¹⁰ is selected from —OR¹² and a C₁₋₆alkyl substituted with —OH.
C9. The compound of any one of embodiments C1-C8, wherein R³ is a 4-6 membered heterocycle that is substituted with one or more R¹⁰, wherein at least one R¹⁰ is an unsubstituted C₁₋₆alkyl.
C10. The compound of any one of embodiments C1-C9, wherein R³ is selected from:

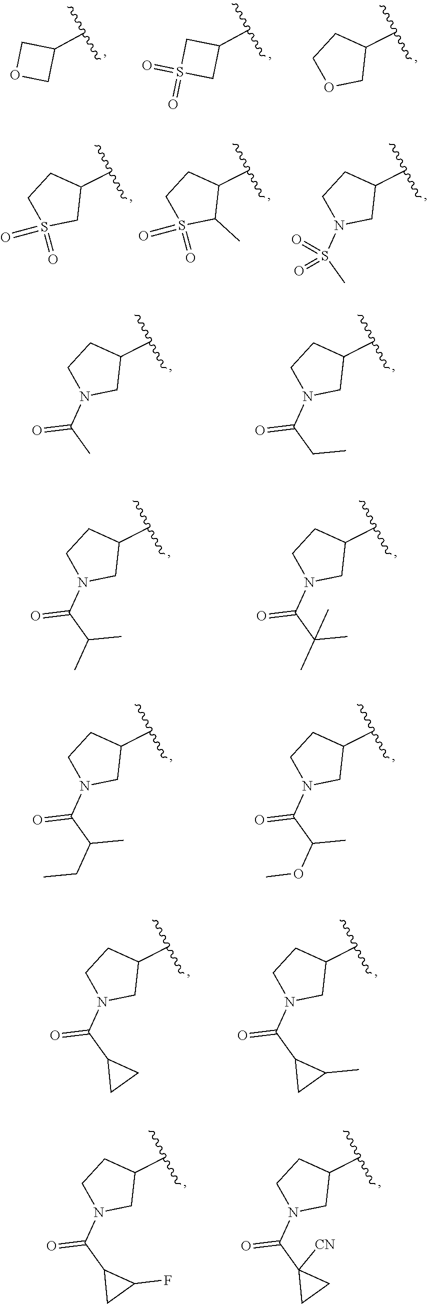

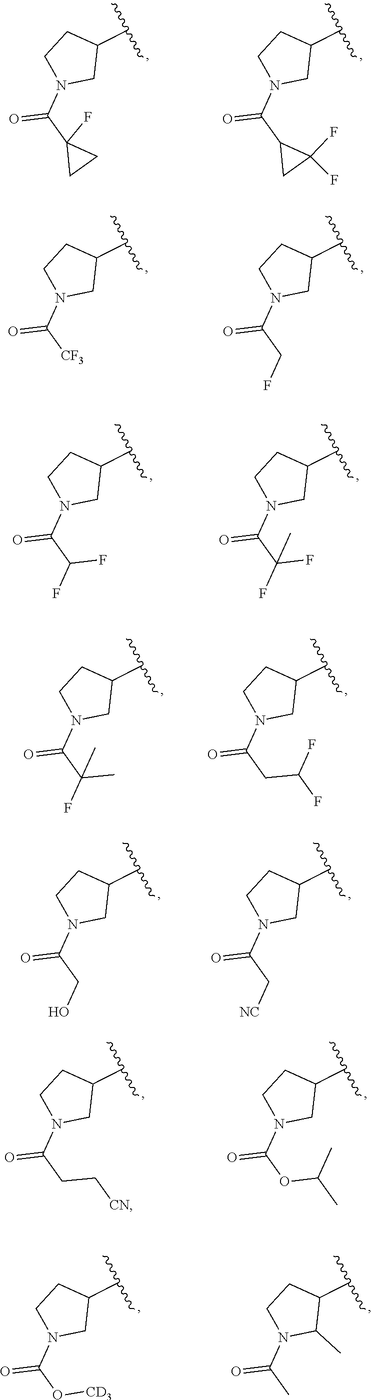

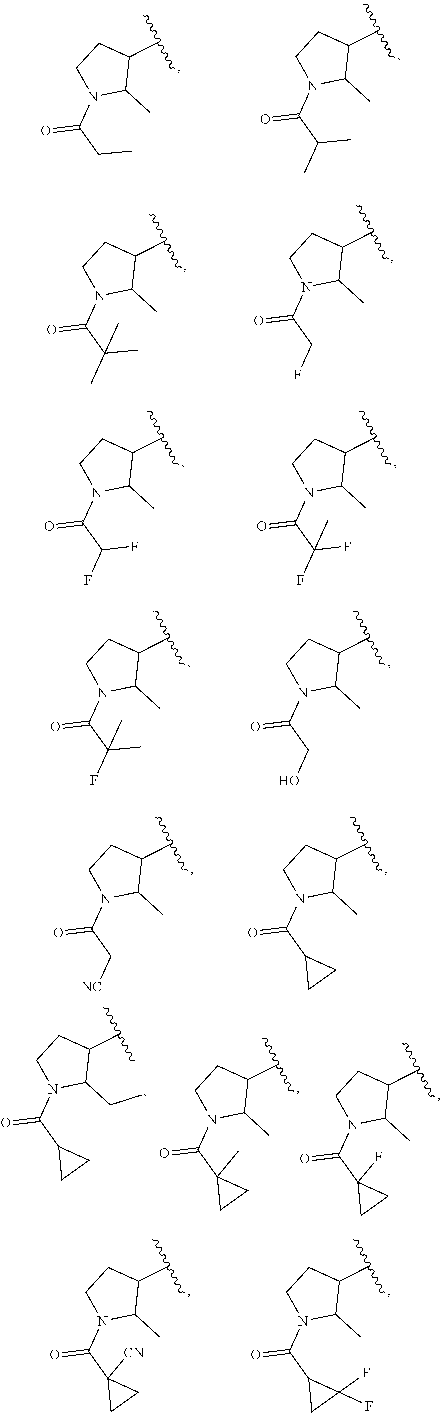

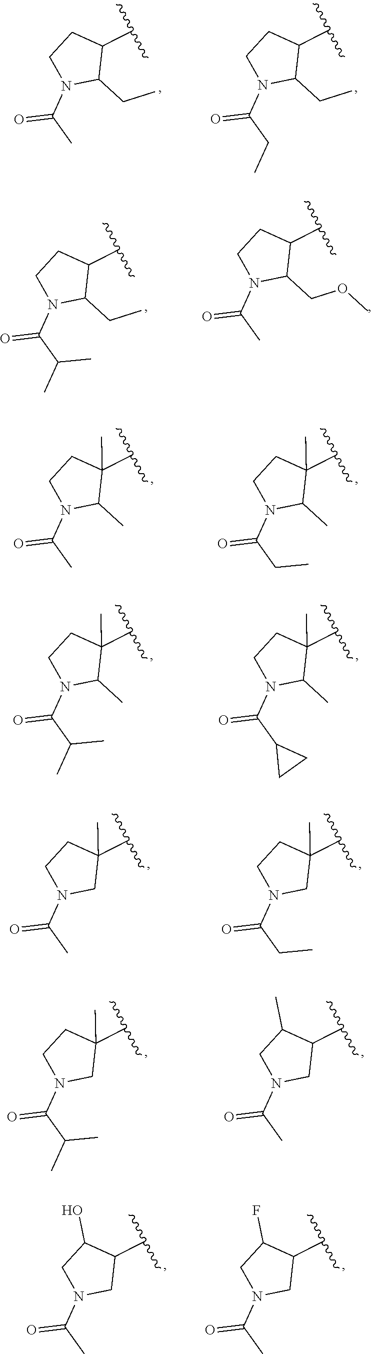

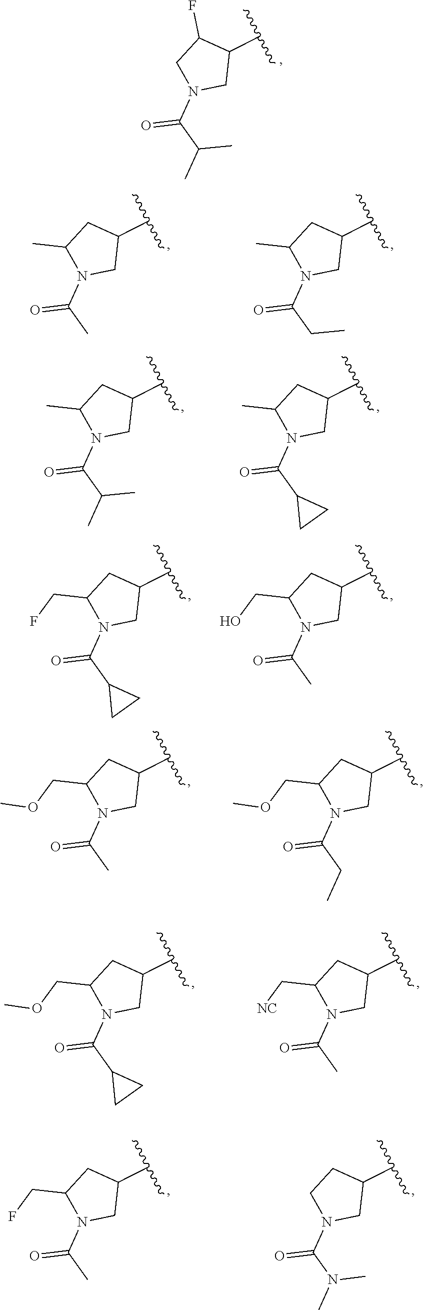

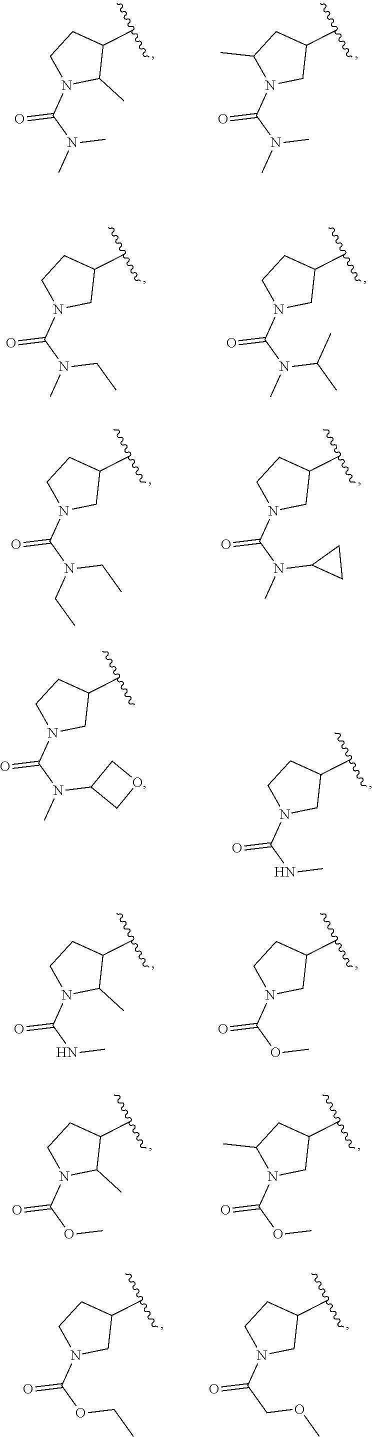

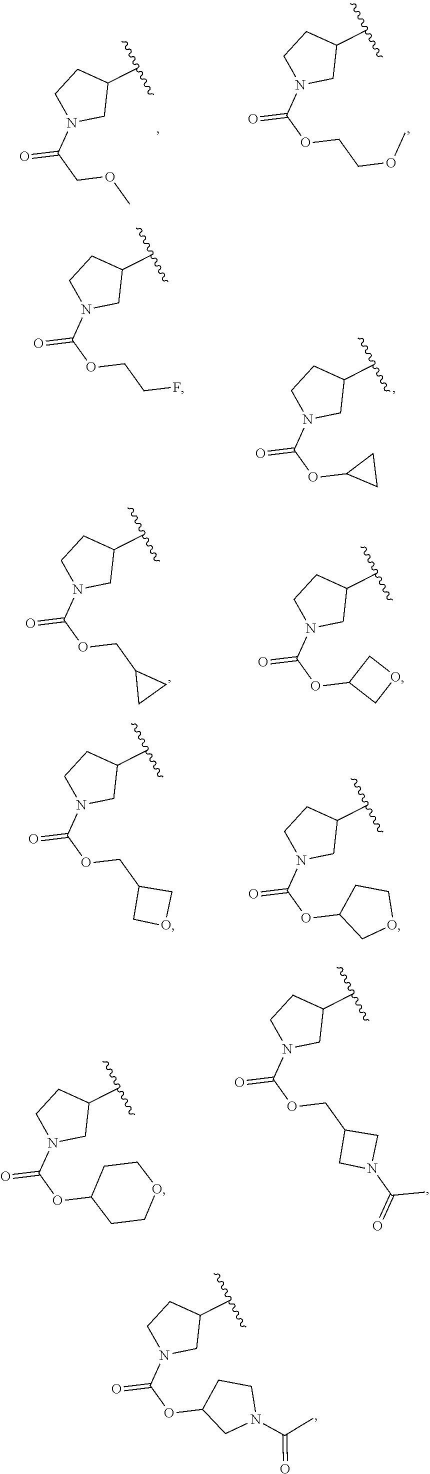

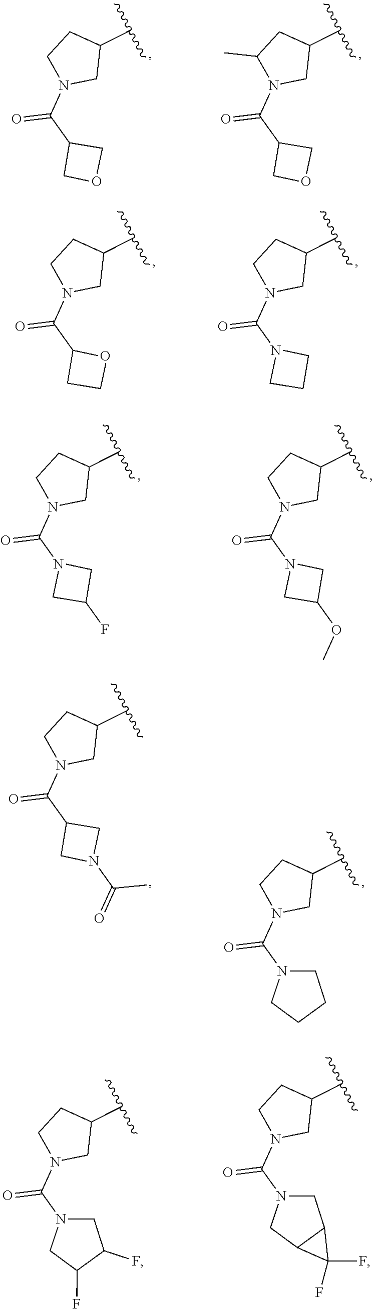

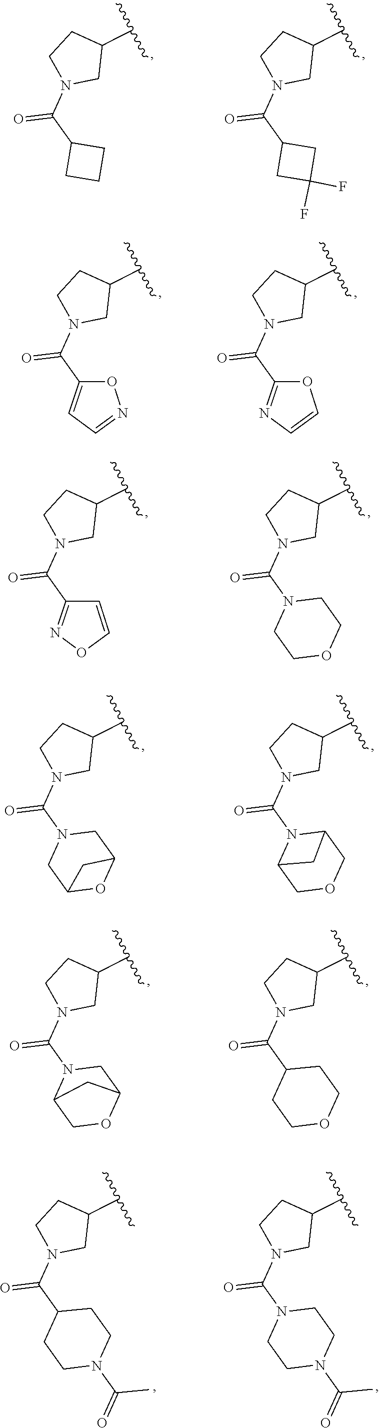

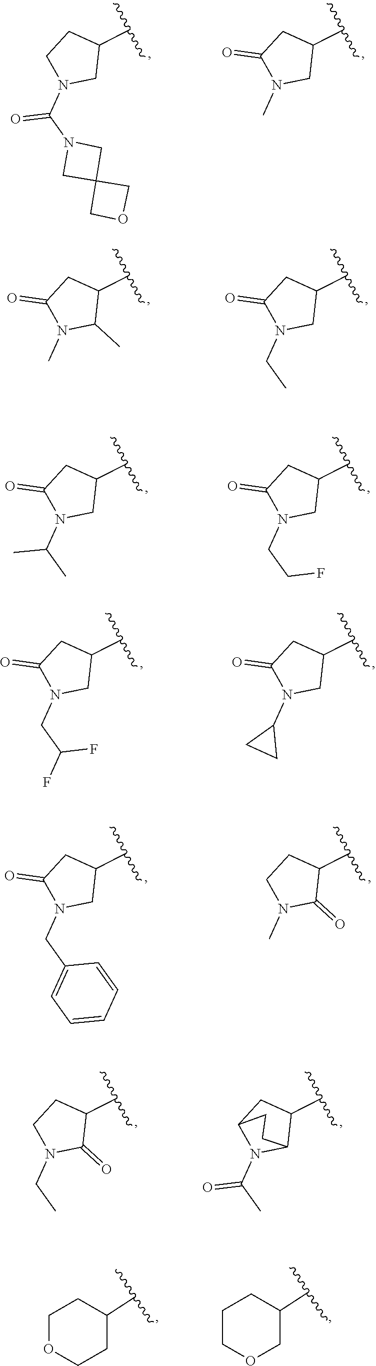

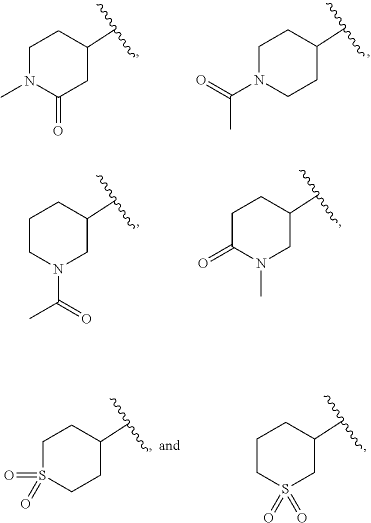

any of which is optionally further substituted with one or more R¹⁰.
C11. The compound of embodiment C1 or C2, wherein R³ is a 7-9 membered heterocycle that includes one or more heteroatoms selected from O, S, and N, wherein the heterocycle is unsubstituted or is substituted with one or more R¹⁰.
C12. The compound of embodiment C11, wherein R³ is a 7-9 membered heterocycle that includes one or more heteroatoms selected from O, S, and N, wherein the heterocycle is unsubstituted or is substituted with one or more R¹⁰, provided that (i) when the heterocycle contains a nitrogen atom, the nitrogen atom is substituted with R¹⁰ or (ii) the heterocycle does not comprise an —NH-moiety.
C13. The compound of embodiment C11 or C12, wherein R³ is a 7-9 membered bridged heterocycle that includes one or more heteroatoms selected from O, S, and N, wherein the bridged heterocycle is unsubstituted or is substituted with one or more R¹⁰.
C14. The compound of embodiment C11 or C12, wherein R³ is a 7-9 membered heterocycle comprising a fused ring system that includes one or more heteroatoms selected from O, S, and N, wherein the heterocycle is unsubstituted or is substituted with one or more R¹⁰.
C15. The compound of any one of embodiments C1, C2, or C11-C14, wherein R³ is selected from:

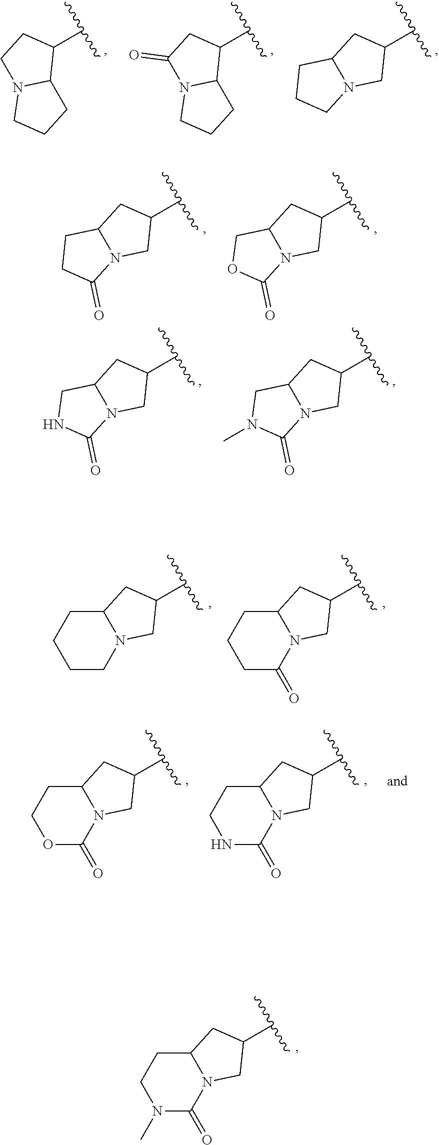

any of which is optionally further substituted with one or more R¹⁰.
C16. The compound of any one of embodiments C1-C15, wherein the compound is a compound according to Formula IA, IIB, IIC, IID, IIE, IIF, IIG, IIH, IIJ, IIK, IIL, IIM, IIN, IIP, IIQ, IIR, IIS, IIT, IIU, IIV, IIW, IIX, IIY, or IIZ:

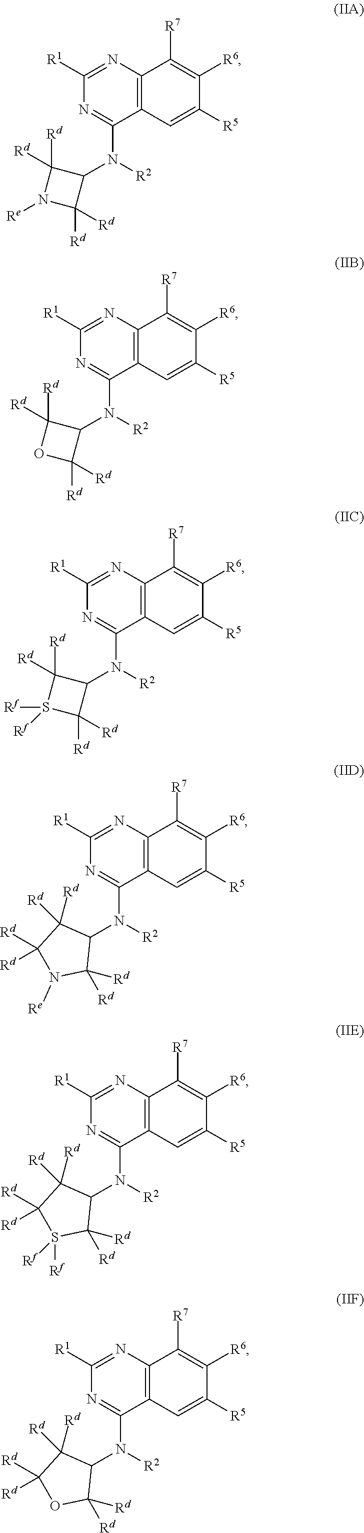

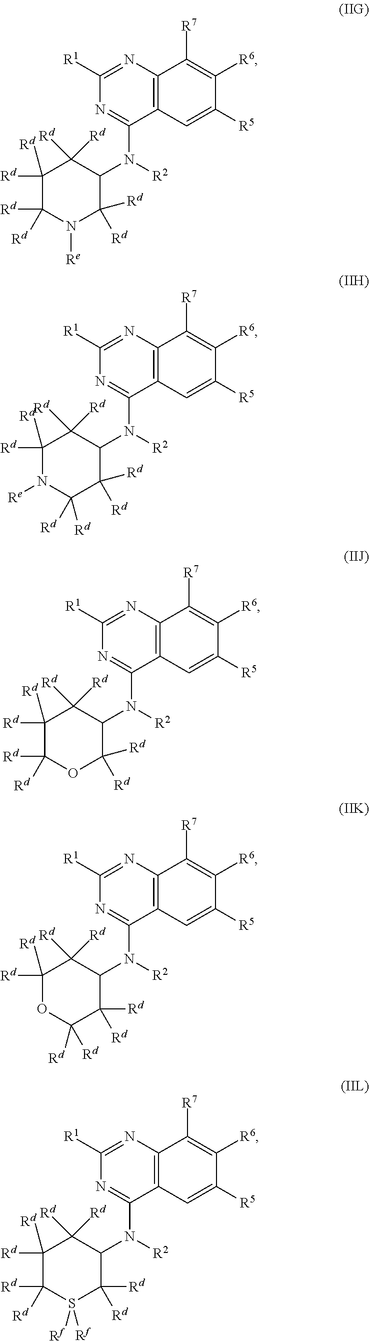

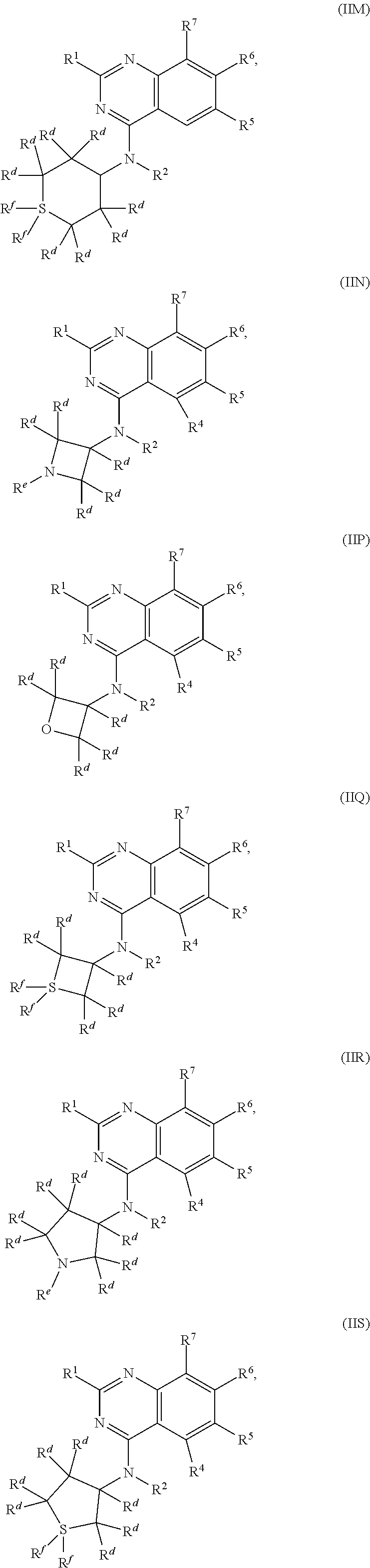

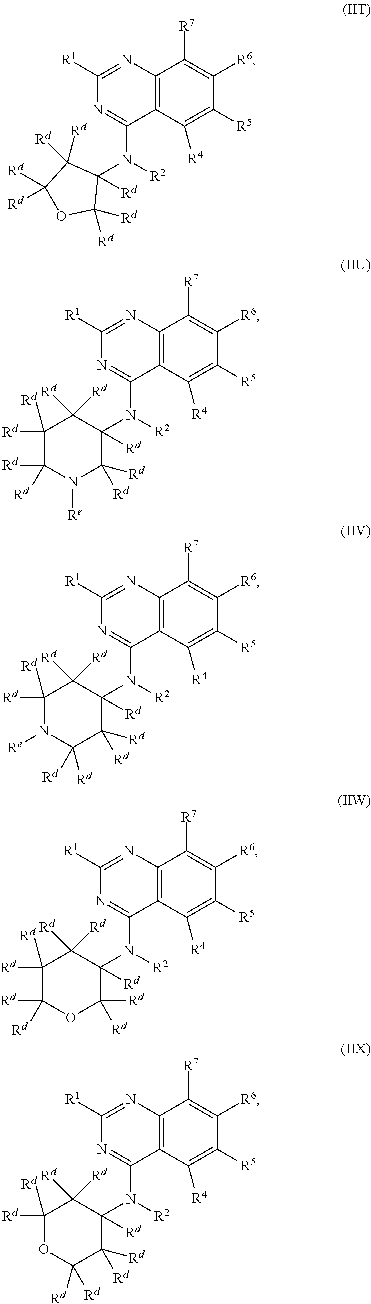

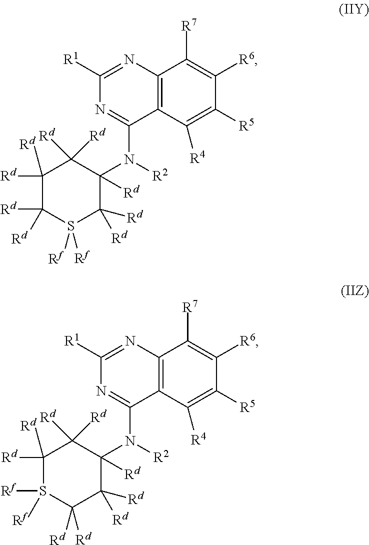

or a salt (e.g., pharmaceutically acceptable salt) thereof, wherein:
each R^{d} is independently selected from H, —OR², ═O, —C(O)C₁₋₆ alkylene)CN, —C(O)C₁₋₆ alkylene)OH, —C(O)(C₁₋₆alkyl), —C(O)N(R¹⁴)₂, —C(O)(3-6 membered carbocycle), —S(O)₂(C₁₋₆alkyl), halogen, and C₁₋₆alkyl, wherein any C₁₋₆alkyl is unsubstituted or substituted with one or more R²⁰, and wherein any 3-6 membered carbocycle is unsubstituted or substituted with one or more R¹² or R²⁰;
R^{e}, if present, is selected from —C(O)(C₁₋₆alkyl)CN, —C(O)C₁₋₆alkyl)OH, —C(O)C₁₋₆alkyl), —C(O)O(C₁₋₆alkyl), —C(O)N(R¹⁴)₂, —C(O)OR¹², —C(O)(3-6 membered carbocycle), —C(O)(3-7 membered heterocycle), —C(O)(5-6 membered heteraryl), —C(O)O(3-6 membered heterocycle), —C(O)O(C₁₋₆ alkylene)(3-6 membered heterocycle), —S(O)₂(C₁₋₆alkyl), a 3-6 membered carbocycle, and C₁₋₆alkyl, wherein any C₁₋₆alkyl is unsubstituted or substituted with one or more R²⁰, and wherein any 3-6 membered carbocycle, 5-6 membered heteroaryl, or 3-6 membered heterocycle is unsubstituted or substituted with one or more R¹² or R²⁰; and
each R^{f} is optionally absent and, if present, is selected from ═O, —NH₂, —NHC₁₋₆alkyl, and —N(C₁₋₆alkyl)₂.
C17. The compound of embodiment C16, wherein the compound is a compound according to Formula IIA, or a salt (e.g., pharmaceutically acceptable salt) thereof.
C18. The compound of embodiment C16, wherein the compound is a compound according to Formula IIB, or a salt (e.g., pharmaceutically acceptable salt) thereof.
C19. The compound of embodiment C16, wherein the compound is a compound according to Formula IIC, or a salt (e.g., pharmaceutically acceptable salt) thereof.
C20 The compound of embodiment C16, wherein the compound is a compound according to Formula IID, or a salt (e.g., pharmaceutically acceptable salt) thereof.
C21. The compound of embodiment C16, wherein the compound is a compound according to Formula IIE, or a salt (e.g., pharmaceutically acceptable salt) thereof.
C22. The compound of embodiment C16, wherein the compound is a compound according to Formula IIF, or a salt (e.g., pharmaceutically acceptable salt) thereof.
C23. The compound of embodiment C16, wherein the compound is a compound according to Formula IIG, or a salt (e.g., pharmaceutically acceptable salt) thereof.
C24. The compound of embodiment C16, wherein the compound is a compound according to Formula IIH, or a salt (e.g., pharmaceutically acceptable salt) thereof.
C25. The compound of embodiment C16, wherein the compound is a compound according to Formula IIJ, or a salt (e.g., pharmaceutically acceptable salt) thereof.
C26. The compound of embodiment C16, wherein the compound is a compound according to Formula IIK, or a salt (e.g., pharmaceutically acceptable salt) thereof.
C27. The compound of embodiment C16, wherein the compound is a compound according to Formula IIL, or a salt (e.g., pharmaceutically acceptable salt) thereof.
C28. The compound of embodiment C16, wherein the compound is a compound according to Formula IIM, or a salt (e.g., pharmaceutically acceptable salt) thereof.
C29. The compound of embodiment C16, wherein the compound is a compound according to Formula IIN, or a salt (e.g., pharmaceutically acceptable salt) thereof.
C30. The compound of embodiment C16, wherein the compound is a compound according to Formula IIP, or a salt (e.g., pharmaceutically acceptable salt) thereof.
C31. The compound of embodiment C16, wherein the compound is a compound according to Formula IIQ, or a salt (e.g., pharmaceutically acceptable salt) thereof.
C32. The compound of embodiment C16, wherein the compound is a compound according to Formula IIR, or a salt (e.g., pharmaceutically acceptable salt) thereof.
C33. The compound of embodiment C16, wherein the compound is a compound according to Formula IIS, or a salt (e.g., pharmaceutically acceptable salt) thereof.
C34. The compound of embodiment C16, wherein the compound is a compound according to Formula IIT, or a salt (e.g., pharmaceutically acceptable salt) thereof.
C35. The compound of embodiment C16, wherein the compound is a compound according to Formula IIU, or a salt (e.g., pharmaceutically acceptable salt) thereof.
C36. The compound of embodiment C16, wherein the compound is a compound according to Formula IIV, or a salt (e.g., pharmaceutically acceptable salt) thereof.
C37 The compound of embodiment C16, wherein the compound is a compound according to Formula IIW, or a salt (e.g., pharmaceutically acceptable salt) thereof.
C38. The compound of embodiment C16, wherein the compound is a compound according to Formula IIX, or a salt (e.g., pharmaceutically acceptable salt) thereof.
C39. The compound of embodiment C16, wherein the compound is a compound according to Formula IIY, or a salt (e.g., pharmaceutically acceptable salt) thereof.
C40. The compound of embodiment C16, wherein the compound is a compound according to Formula IIZ, or a salt (e.g., pharmaceutically acceptable salt) thereof.
C41. The compound of any one of embodiments C1-C15, wherein the compound is a compound according to Formula IIAA:

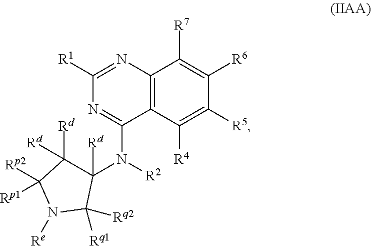

or a salt (e.g., pharmaceutically acceptable salt) thereof, wherein:
each R^{d} is independently selected from H, —OR², ═O, —C(O)(C₁₋₆ alkylene)CN, —C(O)(C₁₋₆ alkylene)OH, —C(O)(C₁₋₆alkyl), —C(O)(3-6 membered carbocycle), —S(O)₂(C₁₋₆alkyl), halogen, and C₁₋₆alkyl, wherein any C₁₋₆alkyl is unsubstituted or substituted with one or more R⁰, wherein any 3-6 membered carbocycle, is unsubstituted or substituted with one or more R² or R¹³; and
(i) R^{q1}, R^{q2}, and R^{p2} are each independently selected from R^{d}, and R^{c} and R^{p1}, together with the atoms to which they are attached, form a 5-6 membered heterocycle that is unsubstituted or is substituted with one or more R^{d}; or
(ii) R^{p1}, R^{P2}, and R^{q2} are each independently selected from R^{d}, and R^{e} and R^{q1}, together with the atoms to which they are attached, form a 5-6 membered heterocycle that is unsubstituted or is substituted with one or more R^{d}.
C42. The compound of any one of embodiments C1-C41, wherein R⁶ is a monocyclic heteroaryl that is substituted with one or more R¹⁵.
C43. The compound of embodiment C42, wherein R⁶ is a pyridine substituted with one or more R¹⁵.
C44. The compound of embodiment C43, wherein R′ has the structure

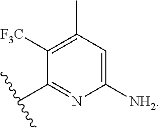 [SMILES: *c1nc(N)cc(C)c1C(F)(F)F]

C45. The compound ofany one of embodiments C1-C41, wherein R⁶ is a bicyclic heteroaryl that is substituted with one or more R¹⁵.

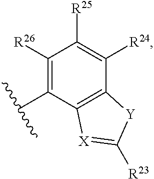

C46. The compound of embodiment C45, wherein R¹ is selected from R²³, wherein:
   X is selected from N and C—CN;
   Y is selected from O and S;
   R²³ is selected from —N(R¹²)₂, C₁₋₆alkyl, and C₁₋₆alkyl-N(R¹⁴)₂, wherein any C₁₋₆akyl is unsubstituted or substituted with one or more R¹³; and
   R²⁴, R²⁵, and R²⁶ are independently selected from H, deuterium, halogen, and C₁₋₆alkyl, wherein any C₁₋₆alkyl is unsubstituted or substituted with one or more R¹³.
C47. The compound of embodiment C45 or C46, wherein R⁶ is selected from:

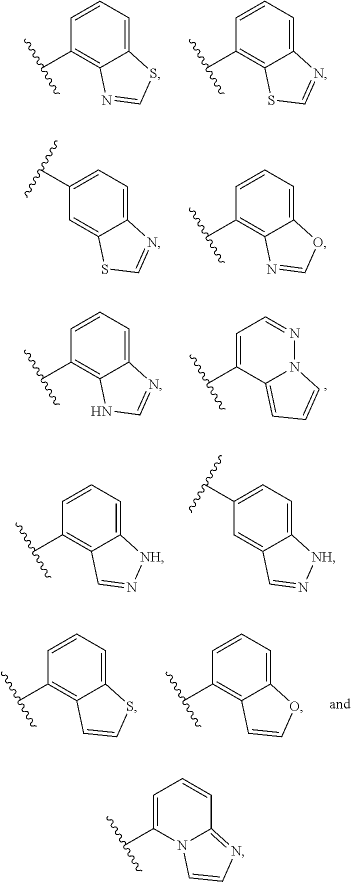

any of which is substituted with one or more R¹⁵.
C48. The compound of any one of embodiments C45-C47, wherein R¹ is selected from:

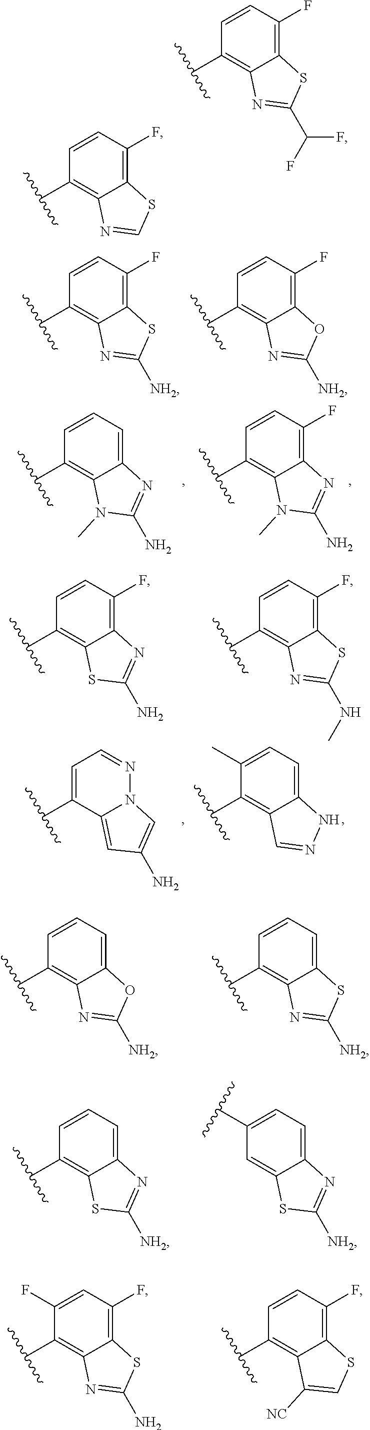

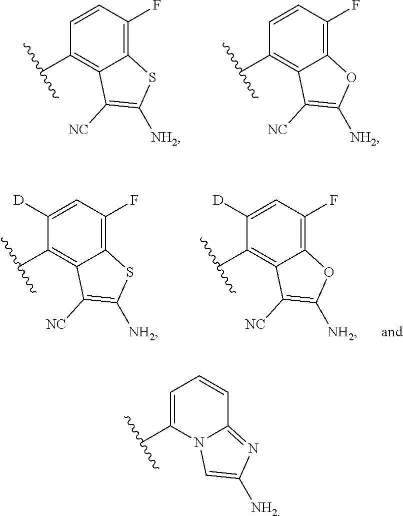 [SMILES: *c1ccc(F)c2sc(N)c(C#N)c12 ; *c1ccc(F)c2oc(N)c(C#N)c12 ; *c1c([2H])cc(F)c2sc(N)c(C#N)c12 ; *c1c([2H])cc(F)c2oc(N)c(C#N)c12 ; *c1cccc2nc(N)cn12]

C49. The compound of an one of embodiments C45-C48, wherein R⁶ is selected from:

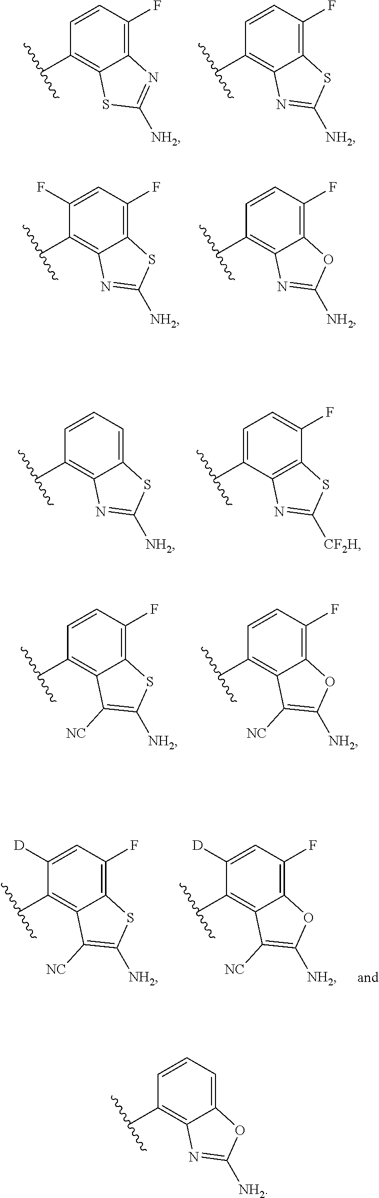

C50. The compound of any one of embodiments C1-C49, wherein the compound is a compound according to Formula IIA1, IIB1, IIC1, IID1, IIE1, IIF1, IIG1, IIH1, IIJ1, IIK1, IIL1, IIM1, IIN1, IIP1, IIQ1, IIR1, IIS1, IIT1, IIU1, IIV1, IIW1, IIX1, IIY1, or IIZ1:

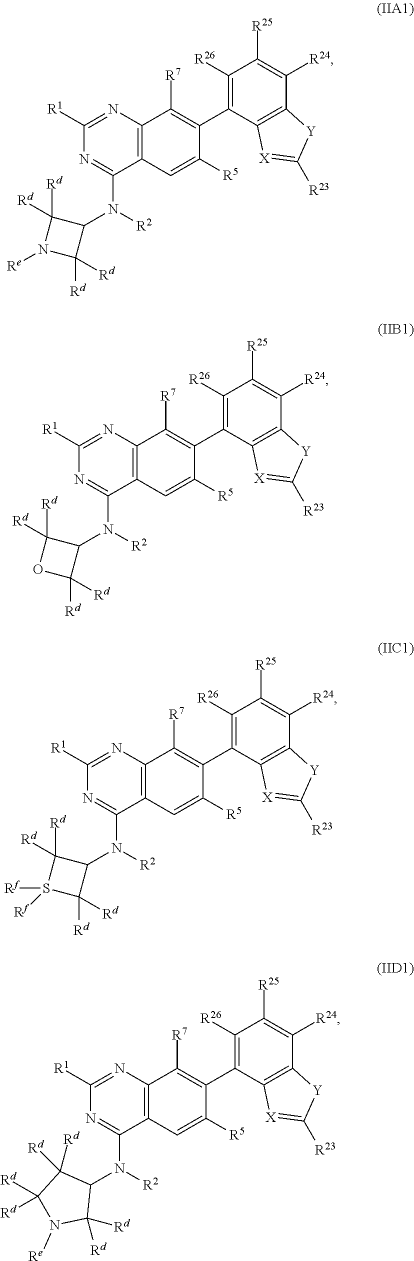

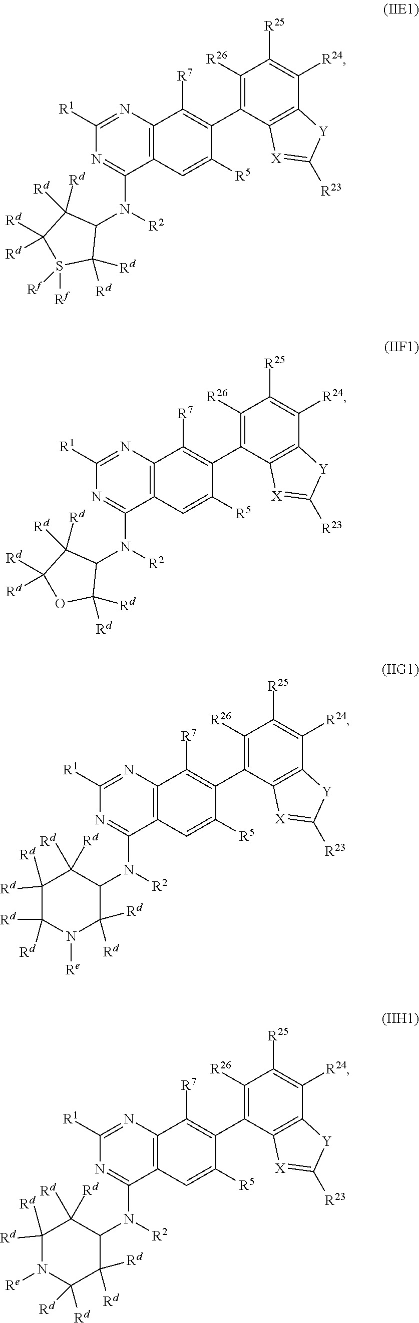

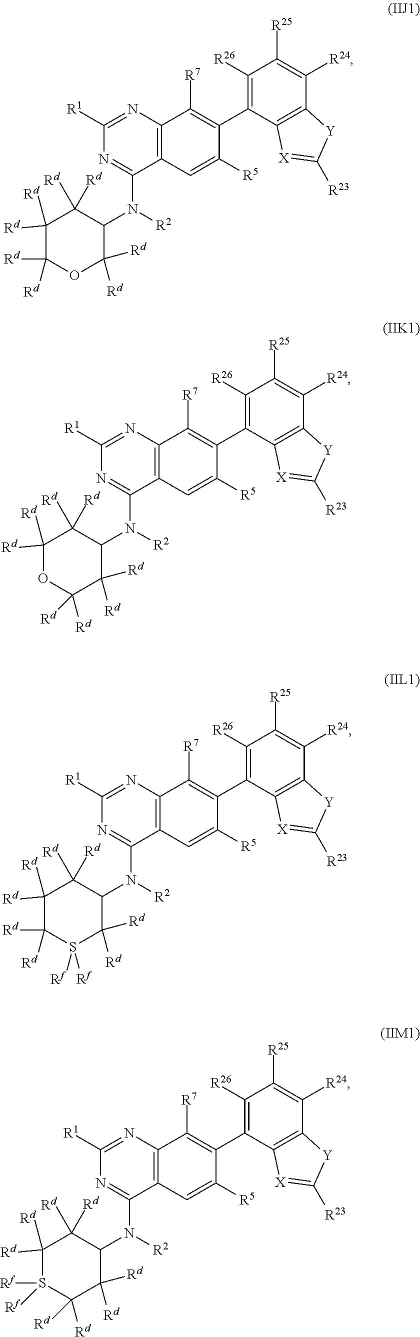

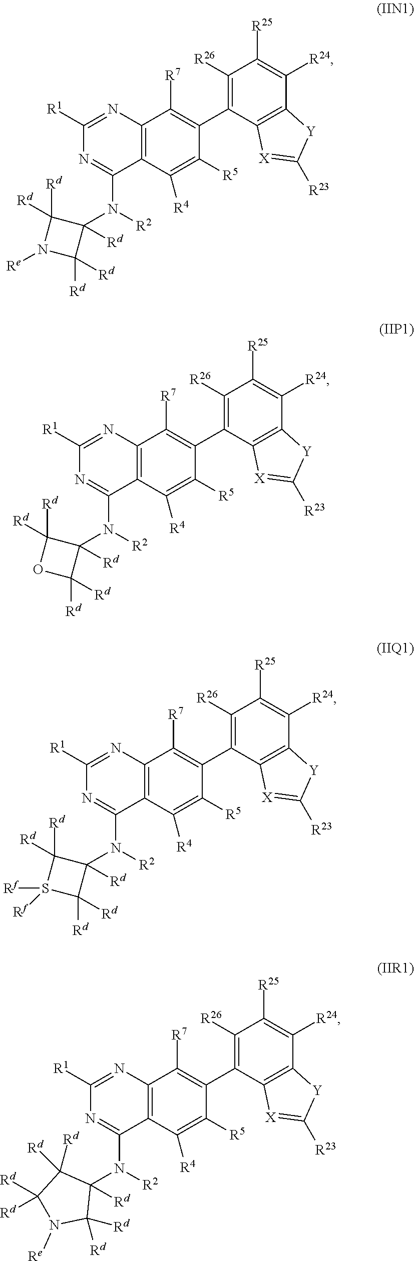

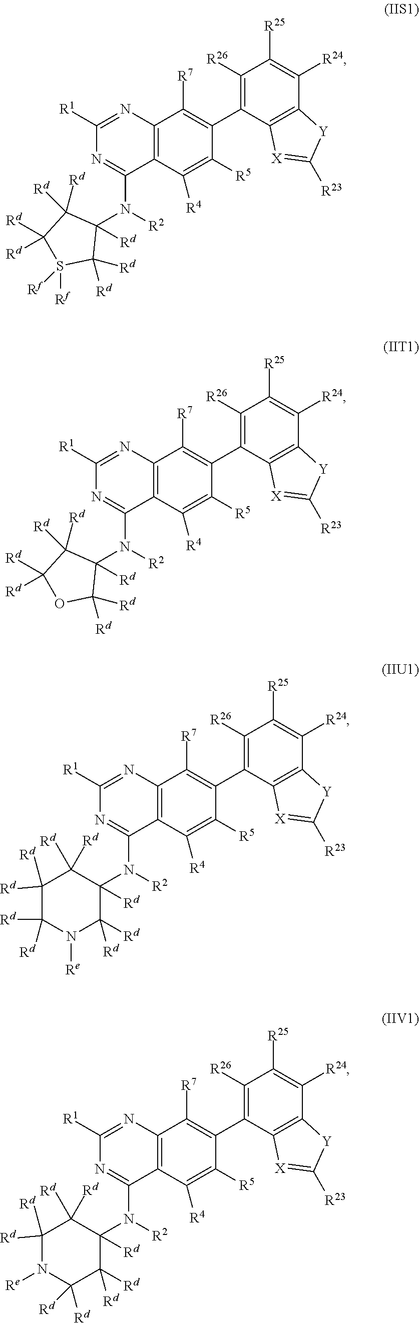

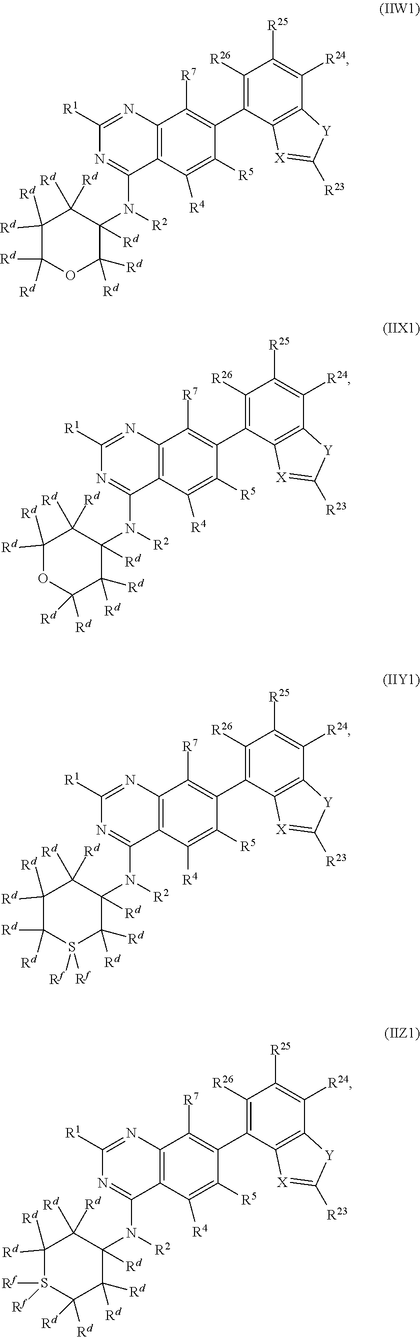

or a salt (e.g., pharmaceutically acceptable salt) thereof, wherein:
each R^{d} is independently selected from H, —OR², ═O, —C(O)(C₁₋₆ alkylene)CN, —C(O)(C₁₋₆ alkylene)OH, —C(O)(C₁₋₆alkyl), —C(O)N(R¹⁴)₂, —C(O)(3-6 membered carbocycle), —S(O)₂(C₁₋₆alkyl), halogen, and C₁₋₆alkyl, wherein any C₁₋₆alkyl is unsubstituted or substituted with one or more R²⁰, and wherein any 3-6 membered carbocycle is unsubstituted or substituted with one or more R¹² or R²⁰;
R^{e}, if present, is selected from —C(O)(C₁₋₆ alkylene)CN, —C(O)(C₁₋₆ alkylene)OH, —C(O)(C₁₋₆alkyl), —C(O)O(C₁₋₆alkyl), —C(O)N(R¹⁴)₂, —C(O)OR¹⁴, —C(O)(3-6 membered carbocycle), —C(O)(3-7 membered heterocycle), —C(O)(5-6 membered heteraryl), —C(O)O(3-6 membered heterocycle), —C(O)O(C₁₋₆ alkylene)(3-6 membered heterocycle), —S(O)₂(C₁₋₆alkyl), a 3-6 membered carbocycle, and C₁₋₆alkyl, wherein any C₁₋₆alkyl is unsubstituted or substituted with one or more R²⁰, and wherein any 3-6 membered carbocycle, 5-6 membered heteroaryl, or 3-6 membered heterocycle is unsubstituted or substituted with one or more R¹² or R²⁰;
each R^{f} is optionally absent and, if present, is selected from ═O, —NH₂, —NHC₁₋₆alkyl, and —N(C₁₋₆alkyl)₂;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, deuterium, halogen, and C₁₋₆alkyl, wherein any C₁₋₆alkyl is unsubstituted or substituted with one or more R¹³.
C51. The compound of embodiment C50, wherein the compound is a compound according to Formula IIA1, or a salt (e.g., pharmaceutically acceptable salt) thereof.
C52. The compound of embodiment C50, wherein the compound is a compound according to Formula IIB1, or a salt (e.g., pharmaceutically acceptable salt) thereof.
C53. The compound of embodiment C50, wherein the compound is a compound according to Formula IIC1, or a salt (e.g., pharmaceutically acceptable salt) thereof.
C54. The compound of embodiment C50, wherein the compound is a compound according to Formula IID1, or a salt (e.g., pharmaceutically acceptable salt) thereof.
C55. The compound of embodiment C50, wherein the compound is a compound according to Formula IIE1, or a salt (e.g., pharmaceutically acceptable salt) thereof.
C56. The compound of embodiment C50, wherein the compound is a compound according to Formula IIF1, or a salt (e.g., pharmaceutically acceptable salt) thereof.
C57. The compound of embodiment C50, wherein the compound is a compound according to Formula IIG1, or a salt (e.g., pharmaceutically acceptable salt) thereof.
C58. The compound of embodiment C50, wherein the compound is a compound according to Formula IIH1, or a salt (e.g., pharmaceutically acceptable salt) thereof.
C59. The compound of embodiment C50, wherein the compound is a compound according to Formula IIJ1, or a salt (e.g., pharmaceutically acceptable salt) thereof.
C60 The compound of embodiment C50, wherein the compound is a compound according to Formula IIK1, or a salt (e.g., pharmaceutically acceptable salt) thereof.
C61. The compound of embodiment C50, wherein the compound is a compound according to Formula IIL1, or a salt (e.g., pharmaceutically acceptable salt) thereof.
C62. The compound of embodiment C50, wherein the compound is a compound according to Formula IIM1, or a salt (e.g., pharmaceutically acceptable salt) thereof.
C63. The compound of embodiment C50, wherein the compound is a compound according to Formula IIN1, or a salt (e.g., pharmaceutically acceptable salt) thereof.
C64. The compound of embodiment C50, wherein the compound is a compound according to Formula IIP1, or a salt (e.g., pharmaceutically acceptable salt) thereof.
C65. The compound of embodiment C50, wherein the compound is a compound according to Formula IIQ1, or a salt (e.g., pharmaceutically acceptable salt) thereof.
C66. The compound of embodiment C50, wherein the compound is a compound according to Formula IIR1, or a salt (e.g., pharmaceutically acceptable salt) thereof.
C67. The compound of embodiment C50, wherein the compound is a compound according to Formula IIS1, or a salt (e.g., pharmaceutically acceptable salt) thereof.
C68. The compound of embodiment C50, wherein the compound is a compound according to Formula IIT1, or a salt (e.g., pharmaceutically acceptable salt) thereof.
C69. The compound of embodiment C50, wherein the compound is a compound according to Formula IIU1, or a salt (e.g., pharmaceutically acceptable salt) thereof.
C70. The compound of embodiment C50, wherein the compound is a compound according to Formula IIV1, or a salt (e.g., pharmaceutically acceptable salt) thereof.
C71. The compound of embodiment C50, wherein the compound is a compound according to Formula IW 1, or a salt (e.g., pharmaceutically acceptable salt) thereof.
C72. The compound of embodiment C50, wherein the compound is a compound according to Formula IIX1, or a salt (e.g., pharmaceutically acceptable salt) thereof.
C73. The compound of embodiment C50, wherein the compound is a compound according to Formula IIY1, or a salt (e.g., pharmaceutically acceptable salt) thereof.
C74. The compound of embodiment C50, wherein the compound is a compound according to Formula IIZ1, or a salt (e.g., pharmaceutically acceptable salt) thereof.
C75. The compound of any one of embodiments C1-C49, wherein the compound is a compound according to Formula IIAA1:

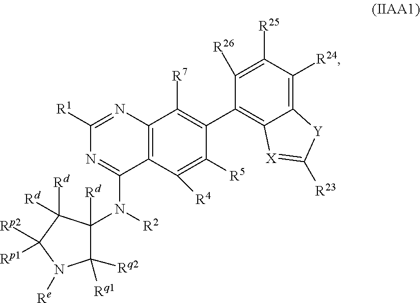

or a salt (e.g., a pharmaceutically acceptable salt) thereof, wherein:
each R^{d} is independently selected from H, —OR², ═O, —C(O)(C₁₋₆ alkylene)CN, —C(O)(C₁₋₆ alkylene)OH, —C(O)(C₁₋₆alkyl), —C(O)(3-6 membered carbocycle), —C(O)N(R¹⁴)₂, —S(O)₂(C₁₋₆alkyl), halogen, and C₁₋₆alkyl, wherein any C₁₋₆alkyl is unsubstituted or substituted with one or more R², and wherein any 3-6 membered carbocycle is unsubstituted or substituted with one or more R¹² or R²⁰;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³;
R²⁴, R²⁵, and R²⁶ are independently selected from H, deuterium, halogen, and C₁₋₆alkyl, wherein any C₁₋₆alkyl is unsubstituted or substituted with one or more R¹³; and
(i) R^{q1}, R^{q2}, and R^{p2} are each independently selected from R^{d}, and R^{e} and R^{p1}, together with the atoms to which they are attached, form a 5-6 membered heterocycle that is unsubstituted or is substituted with one or more R^{d}; or
(ii) R^{p1}, R^{p2}, and R^{q2} are each independently selected from R^{d}, and R^{e} and R^{q1}, together with the atoms to which they are attached, form a 5-6 membered heterocycle that is unsubstituted or is substituted with one or more R^{d}.
C76. The compound of any one of embodiments C50-C75, wherein X is C—CN, Y is S, and R is —N(R¹²)₂.
C77. The compound of any one of embodiments C50-C76, wherein one or more of R²⁴, R²⁵, and R²⁶ is a halogen (e.g., F).
C78. The compound ofany one of embodiments C19, C21, C27, C28, C31, C33, C39, C40, C53, C55, C61, C62, C65, C67, C73, and C74, wherein each R^{f} is ═O.
C79. The compound ofany one of embodiments C19, C21, C27, C28, C31, C33, C39, C40, C53, C55, C61, C62, C65, C67, C73, and C74, wherein each R^{f} is absent.
C80. The compound of any one of embodiments C17. C20, C23, C24, C32. C35, C36, C41, C51, C54, C57, C58, C63, C66, C69, C70, and C75, wherein R¹ is C₁₋₆alkyl that is unsubstituted or substituted with one or more R²⁰.
C81. The compound of any one of embodiments C17, C20, C23, C24, C32, C35, C36, C41, C51, C54, C57, C58, C63, C66, C69, C70, and C75, wherein R^{e} is —C(O)(3-6 membered carbocycle).
C82. The compound of any one of embodiments C17-C81, wherein each R^{d} is H.
C83. The compound of any one of embodiments C17-C81, wherein at least one R^{d} is selected from —OR¹² and a C₁₋₆alkyl substituted with —OH.
C84. The compound of any one of embodiments C1-C83, wherein R² is H.
C85. The compound of any one of embodiments C1-C83, wherein R² is selected from C₁₋₆alkyl that is unsubstituted or is substituted with one or more R¹³.
C86. The compound of embodiment C85, wherein R² is selected from C₁₋₂alkyl.
C87. The compound of any one of embodiments C1-C83, wherein R² is selected from a 3-6 membered carbocycle.
C88. The compound of any one of embodiments C1-C87, wherein R¹ is selected from —OR⁸.
C89. The compound of embodiment C88, wherein R¹ is selected from:

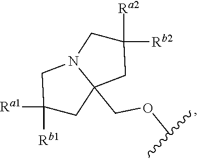

wherein R^{a1}, R^{a2}, R^{b1}, and R^{b2} are each independently selected from deuterium, halogen, C₁₋₆alkyl, —OR², and H, wherein R^{a2} and R^{b2} can optionally join together to form a 3-6 membered carbocycle, and wherein any C₁₋₆alkyl or 3-6 membered carbocycle is unsubstituted or is substituted with one or more R¹³.
C90. The compound of embodiment C89, wherein R¹ is selected from:

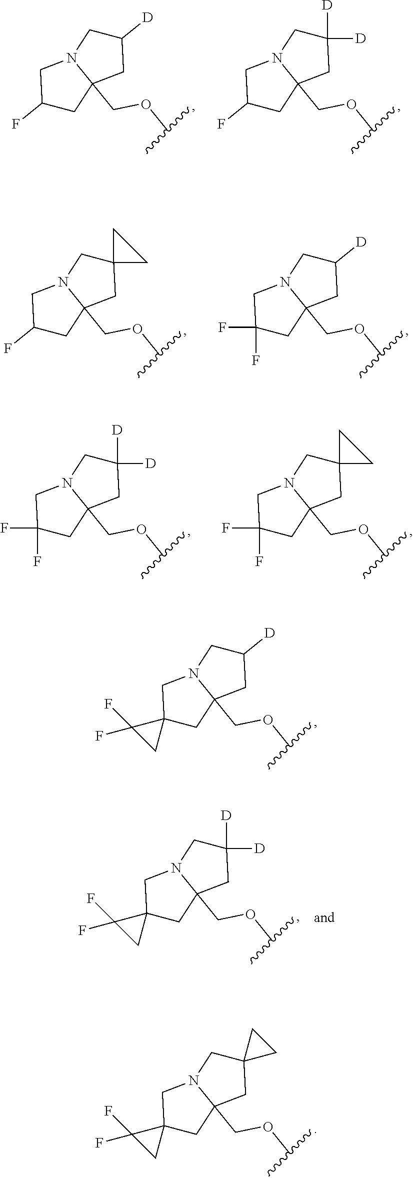

C91. The compound of embodiment C88, wherein R¹ is selected from:

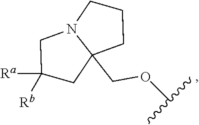

wherein R^{a} and R^{b} are each independently selected from halogen, C₁₋₆alkyl, —OR², and H, wherein any C₁₋₆alkyl is unsubstituted or is substituted with one or more R¹³.
C92. The compound of embodiment C91, wherein R¹ is selected from:

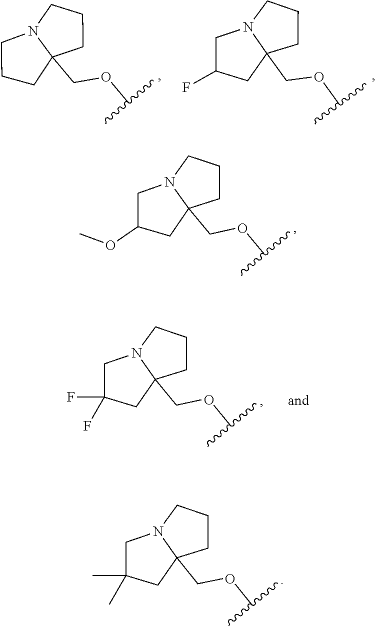 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

C93. The compound of embodiment C88, wherein R¹ is selected from:

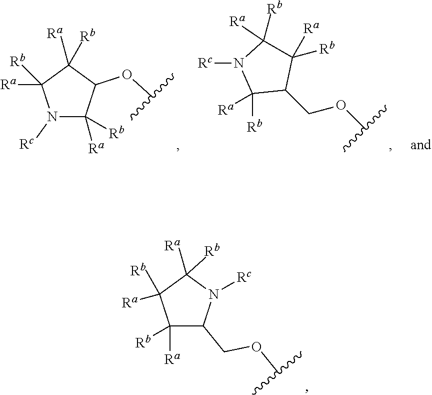

wherein each R^{a} and R^{b} is independently selected from halogen, C₁₋₆ alkyl, —OR¹², and H; and R′ is selected from C₁₋₆ alkyl, wherein an R^{a} and R^{b} or R^{c} optionally join together to form a 3-6 membered carbocycle or heterocycle, and wherein any C₁₋₆ alkyl or 3-6 membered carbocycle or heterocycle is unsubstituted or is substituted with one or more R¹³.
C94 The compound of embodiment C93, wherein R¹ is selected from

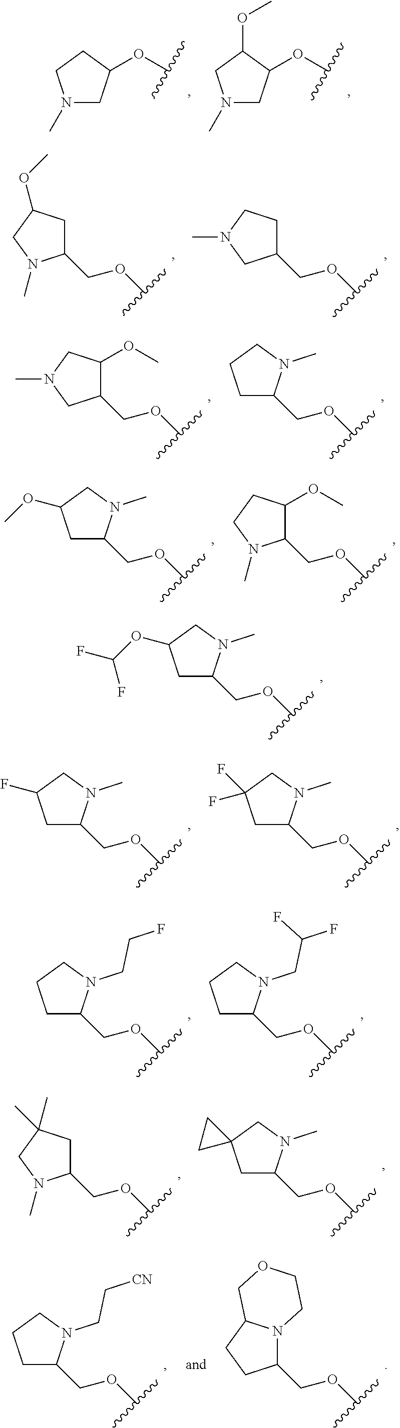

C95. The compound of any one of embodiments C1-C87, wherein R¹ is selected from

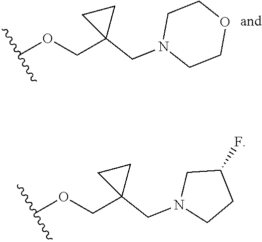 [SMILES: *OCC1(CN2CCOCC2)CC1 ; *OCC1(CN2CC[C@@H](F)C2)CC1]

C96. The compound of any one of embodiments C1-C95, wherein R⁵ is H.
C97. The compound of any one of embodiments C1-C95, wherein R⁵ is a halogen (e.g., F or Cl).
C98. The compound of any one of embodiments C1-C95, wherein R³ is selected from C₁₋₆alkyl that is unsubstituted or substituted with one or more R¹³.
C99. The compound of embodiment C98, wherein R⁵ is selected from C₁₋₆alkyl that is substituted with one or more halogens or —CN.
C100. The compound of embodiment C99, wherein R⁵ is selected from —CF₂H, —CF₃, —CH₂CN, and —CH₂CH₃.
C101. The compound of embodiment C100, wherein R⁵ is —CF₃.
C102. The compound of any one of embodiments C1-C101, wherein the compound is a not a compound included in Table 1.
C103. A compound represented by Formula III′:

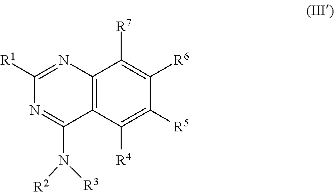

or a salt (e.g., pharmaceutically acceptable salt) thereof, wherein:
R¹ is selected from —OR⁸ and

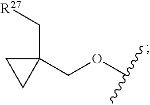

R² and R³, together with the nitrogen atom to which they are attached, form a 4-10 membered heterocycle that is unsubstituted or is substituted with one or more R¹³;
R⁴ is H;
R⁵ is selected from halogen and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹¹ is independently selected from deuterium, —OR¹², ═O, =N(R¹⁴), —C(O)(C₁₋₆ alkylene)CN, —C(O)(C₁₋₆ alkylene)OR¹², —C(O)C₁₋₆alkyl), —C(O)N(R¹⁴)₂, —C(O)(3-6 membered carbocycle or heterocycle), —S(O)₂(C₁₋₆alkyl), —N(R¹⁴)C(O)(C₁₋₆alkyl), —N(R¹⁴)C(O)(C₁₋₆ alkylene)OR¹⁴, halogen, —CN, a 3-6 membered carbocycle or heterocycle, and C₁₋₆alkyl, wherein any C₁₋₆alkyl, carbocycle, or heterocycle is unsubstituted or substituted with one or more R,
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₄ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹³;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen; and
R^{a} and R^{b} are each independently selected from deuterium, halogen, C₁₋₆ alkyl, a 3-6 membered carbocycle, —OR¹², and H, wherein an R and R optionally join together to form a 3-6 membered carbocycle or heterocycle, and wherein any C₁₋₆alkyl or 3-6 membered carbocycle or heterocycle is unsubstituted or is substituted with one or more R¹³.
C104. A compound represented by Formula III:

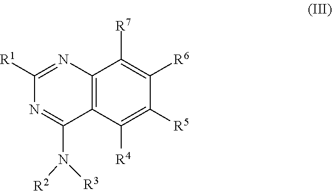

or a salt (e.g., pharmaceutically acceptable salt) thereof, wherein:

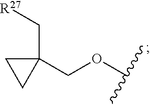

R¹ is selected from —OR⁸ and;
R² and R³, together with the nitrogen atom to which they are attached, form a 4-10 membered heterocycle that is unsubstituted or is substituted with one or more R¹¹, provided that when the heterocycle contains an additional nitrogen atom, the additional nitrogen atom is substituted with R¹³;
R⁴ is H;
R⁵ is selected from halogen and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety ofany alkylheterocycle is selected from C₁₋₆alkyl;
each R¹¹ is independently selected from deuterium, —OR¹², ═O, =N(R¹⁴), —C(O)(C₁₋₆ alkylene)CN, —C(O)C₁₋₆ alkylene)OR¹², —C(O)C₁₋₆alkyl), —C(O)N(R¹⁴)₂, —C(O)(3-6 membered carbocycle or heterocycle), —S(O)₂(C₁₋₆alkyl), —N(R¹⁰)C(O)(C₁₋₆alkyl), —N(R¹⁴)C(O)(C₁₋₆ alkylene)OR¹⁴, halogen, —CN, a 3-6 membered carbocycle or heterocycle, and C₁₋₆alkyl, wherein any C₁₋₆alkyl, carbocycle, or heterocycle is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹³;
each R²⁰ is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R¹³;
each R²⁸ is independently selected from C₁₋₆alkyl and halogen; and
R^{a} and R^{b} are each independently selected from deuterium, halogen, C₁₋₆ alkyl, a 3-6 membered carbocycle, —OR², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.
C105. The compound of embodiment C103 or C104, wherein R² and R³, together with the nitrogen atom to which they are attached, form a 4-7 membered heterocycle.
C106. The compound of embodiment C105, wherein R² and R³, together with the nitrogen atom to which they are attached, form a 4-7 membered heterocycle that includes 1 or 2 heteroatoms selected from O, S, and N.
C107. The compound of embodiment C106, wherein R² and R³, together with the nitrogen atom to which they are attached, form a 4-7 membered heterocycle that includes 1 or 2 heteroatoms selected from O, S, and N, wherein the heterocycle is substituted with 1-4 R¹¹.
C108. The compound of any one of embodiments C103-C107, wherein R² and R³, together with the nitrogen atom to which they are attached, form a pyrrolidine that is substituted with 0-4 R¹¹.
C109. The compound of any one of embodiments C103-C107, wherein R² and R³, together with the nitrogen atom to which they are attached, form an azetidine that is substituted with 0-4 R¹¹.
C110. The compound of any one of embodiments C103-C107, wherein R² and R³, together with the nitrogen atom to which they are attached, form a piperidine that is substituted with 0-4 R¹¹.
C111. The compound of any one of embodiments C103-C107, wherein R² and R³, together with the nitrogen atom to which they are attached, form a piperazine that is substituted with 0-4 R¹¹.
C112. The compound of any one of embodiments C103-C107, wherein R² and R³, together with the nitrogen atom to which they are attached, form a morpholine or thiomorpholine that is substituted with 0-4 R¹¹.
C113. The compound ofany one of embodiments C103-C107, wherein R² and R³, together with the nitrogen atom to which they are attached, form a 7-membered heterocycle that includes 1-3 heteroatoms selected from O, S, and N.
C114. The compound of any one of embodiments C103-C113, wherein R² and R³, together with the nitrogen atom to which they are attached, form a 4-7 membered heterocycle substituted with one or more R¹¹, wherein at least one R¹¹ is selected from —OR¹¹ and a C₁₋₆alkyl substituted with —OH.
C115. The compound of any one of embodiments C103-C114, wherein R² and R³, together with the nitrogen atom to which they are attached, form a 4-7 membered heterocycle substituted with one or more R¹¹, wherein at least one R¹¹ is an unsubstituted C₁₋₆alkyl.
C116. The compound of any one of embodiments C103-C113, wherein R² and R³, together with the nitrogen atom to which they are attached, form a 4-7 membered heterocycle that is unsubstituted.
C117. The compound of embodiment C103 or C104, wherein R² and R³, together with the nitrogen atom to which they are attached, form a bridged heterocycle.
C118. The compound of embodiment C117, wherein R² and R³, together with the nitrogen atom to which they are attached, form a 7-9 membered bridged heterocycle that includes 1 or 2 heteroatoms selected from O, S, and N.
C119. The compound of embodiment C118, wherein R² and R³, together with the nitrogen atom to which they are attached, form a 7-9 membered bridged heterocycle that includes 1 or 2 heteroatoms selected from O, S, and N, wherein the bridged heterocycle is substituted with 1-4 R¹¹.
C120. The compound of any one of embodiments C117-C119, wherein R² and R³, together with the nitrogen atom to which they are attached, form a bridged piperidine that is substituted with 0-4 R¹¹.
C121. The compound of any one of embodiments C117-C119, wherein R² and R³, together with the nitrogen atom to which they are attached, form a bridged piperazine that is substituted with 0-4 R¹¹.
C122. The compound of any one of embodiments C117-C119, wherein R² and R³, together with the nitrogen atom to which they are attached, form a bridged morpholine or thiomorpholine that is substituted with 0-4 R¹¹.
C123. The compound of any one of embodiments C117-C122, wherein R² and R³, together with the nitrogen atom to which they are attached, form a bridged heterocycle substituted with one or more R¹¹, wherein at least one R¹¹ is selected from —OR¹² and a C₁₋₆alkyl substituted with —OH.
C124. The compound of any one of embodiments C117-C123, wherein R² and R³, together with the nitrogen atom to which they are attached, form a bridged heterocycle substituted with one or more R¹¹, wherein at least one R¹¹ is an unsubstituted C₁₋₆alkyl.
C125. The compound of any one of embodiments C117-C122, wherein R² and R³, together with the nitrogen atom to which they are attached, form a bridged heterocycle that is unsubstituted.
C126. The compound of embodiment C103 or C104, wherein R² and R³, together with the nitrogen atom to which they are attached, form a spirocycle.
C127. The compound of embodiment C103 or C104, wherein R² and R³, together with the nitrogen atom to which they are attached, form a fused ring system including at least two rings.
C128. The compound of any one of embodiments C103-C115, C117-C124, C126, and C127, wherein R² and R³, together with the nitrogen atom to which they are attached, form a 4-10 membered heterocycle that is substituted with one or more R¹¹, wherein the one or more R¹¹ are independently selected from deuterium, —OR¹², ═O, =N(R¹⁴), —C(O)(C₁₋₆ alkylene)CN, —C(O)C₁₋₆ alkylene)OR¹⁴, —C(O)(C₁₋₆alkyl), —C(O)N(R¹⁴)₂, —S(O)₂(C₁₋₆alkyl), —N(R¹⁴)C(O)C₁₋₆alkyl), —N(R¹⁴)C(O)(C₁₋₆ alkylene)OR¹⁴, halogen, and C₁₋₆alkyl that is unsubstituted or substituted with one or more R²⁰.
C129. The compound of embodiment C128, wherein R² and R³, together with the nitrogen atom to which they are attached, form a 4-10 membered heterocycle that is substituted with one or more R¹¹, wherein the one or more R¹¹ are independently selected from —OR¹², —C(O)(C₁₋₆ alkylene)CN, —C(O)(C₁₋₆ alkylene)OR¹⁴, —C(O)(C₁₋₆alkyl), —C(O)N(R¹⁴)₂, —N(R¹⁴)C(O)(C₁₋₆alkyl), —N(R¹⁴)C(O)(C₁₋₆ alkylene)OR¹⁴, halogen, and C₁₋₆alkyl that is unsubstituted or substituted with one or more R².
C130. The compound of any one of embodiments C103-C129, wherein R² and R³, together with the nitrogen atom to which they are attached, form a structure selected from:

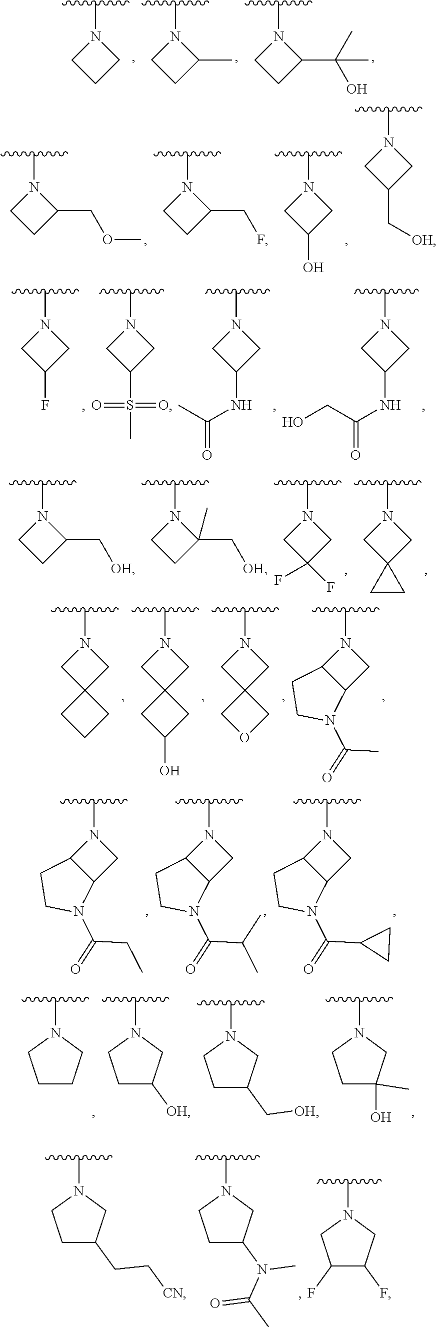

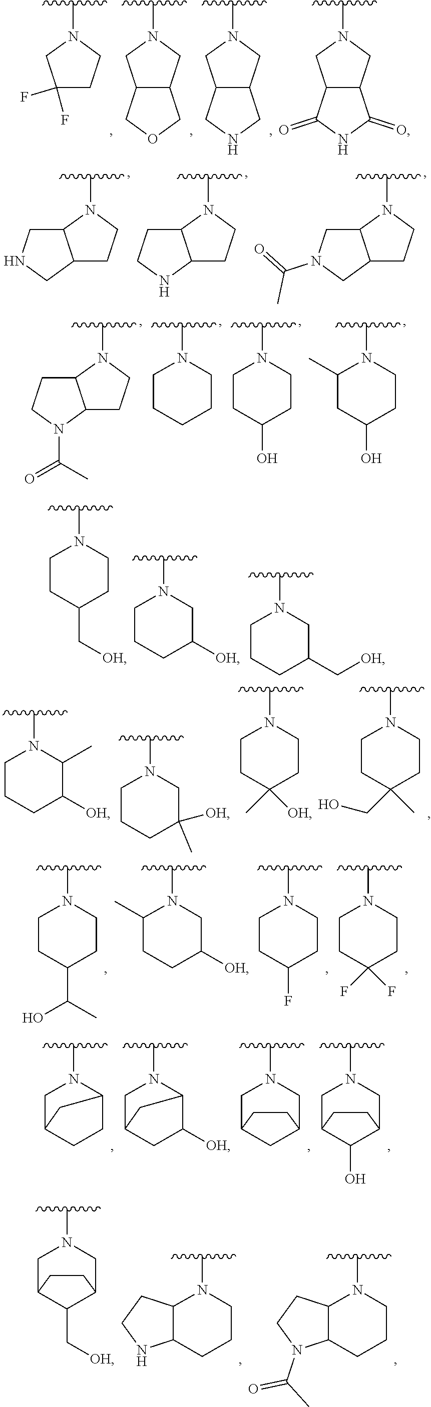

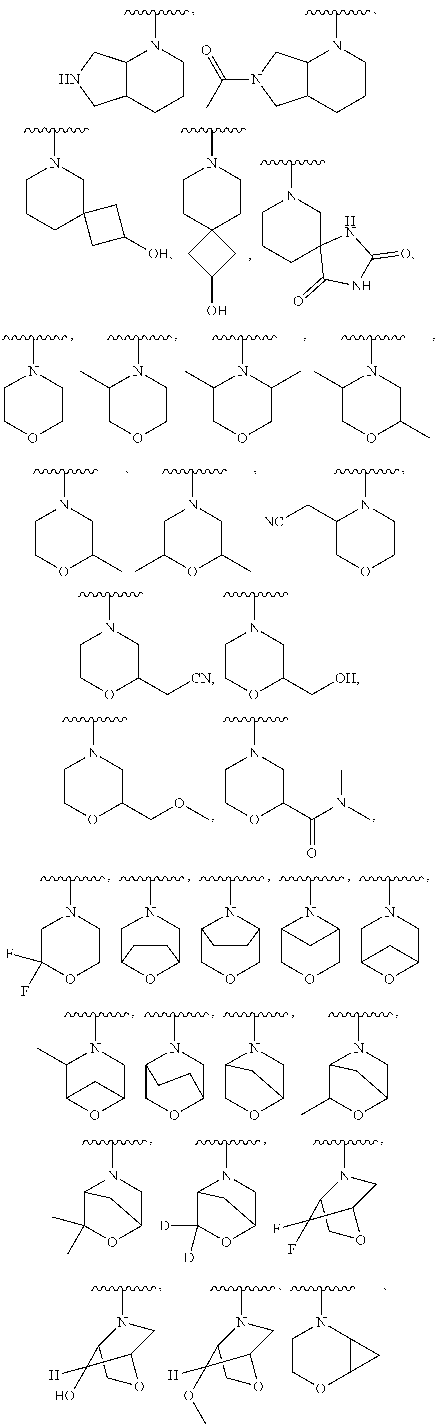

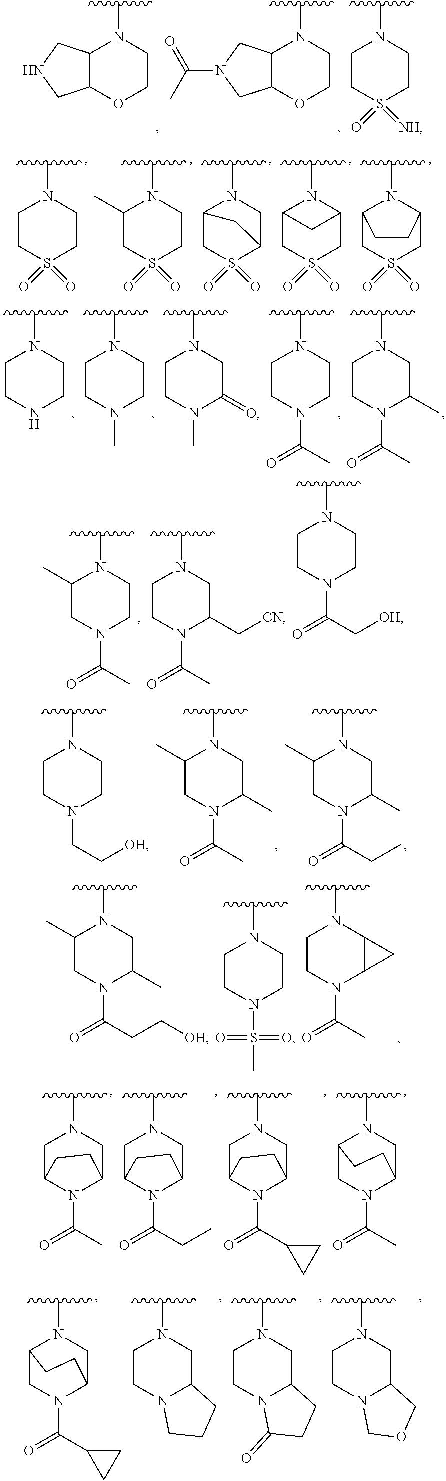

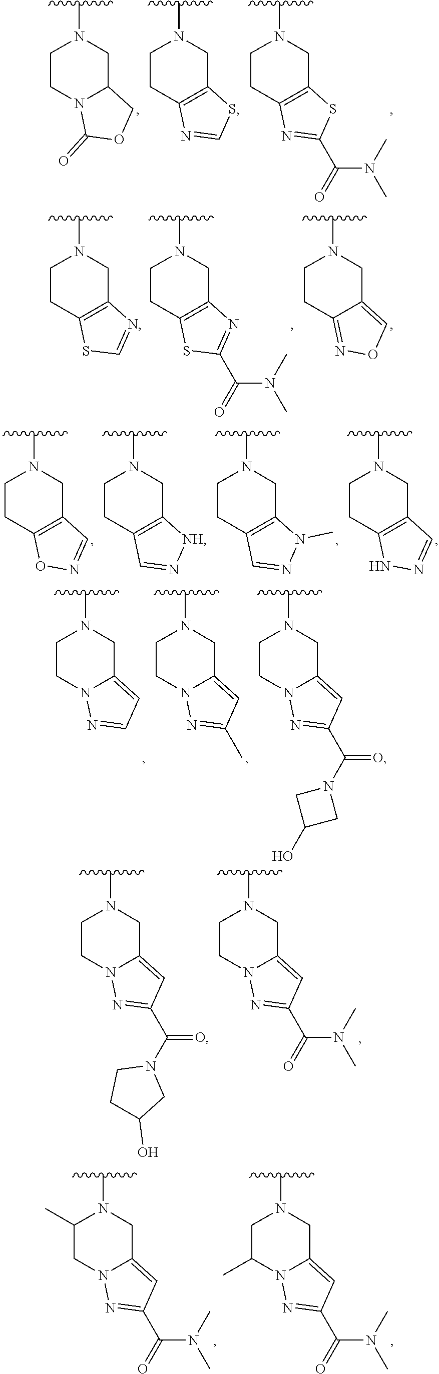

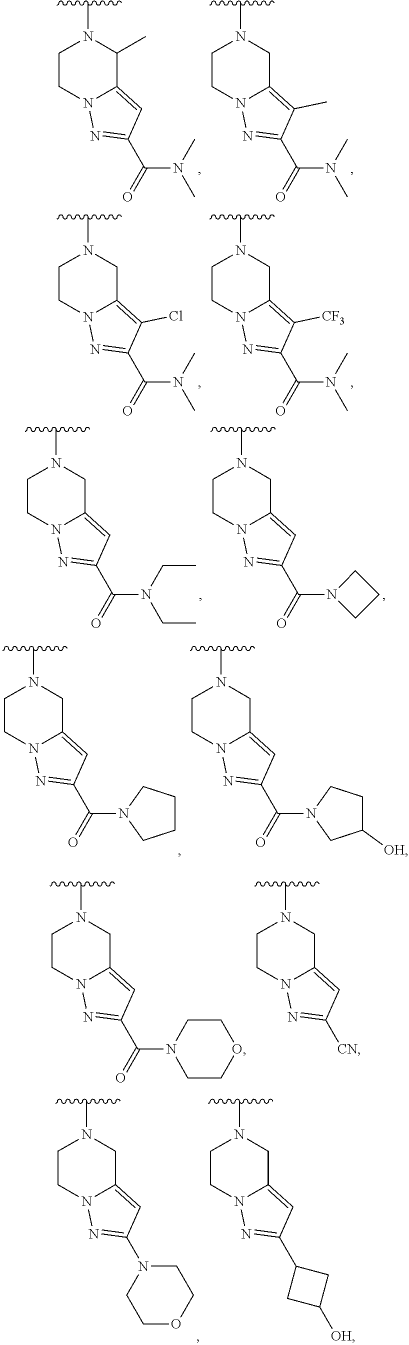

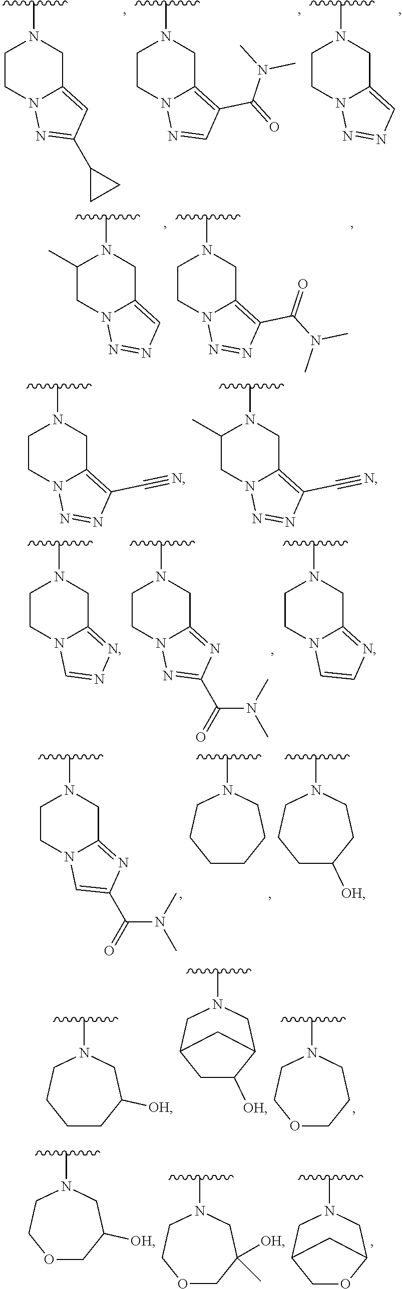

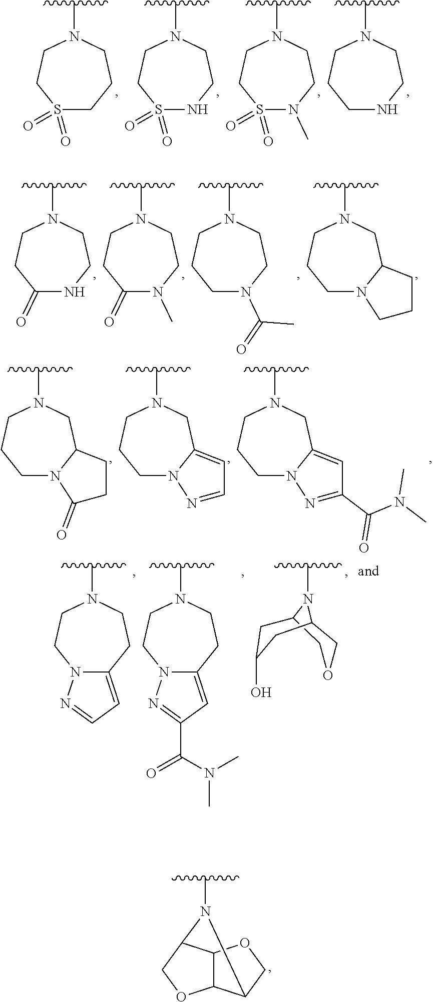

any of which is optionally further substituted with one or more R¹³.
C131. The compound of any one of embodiments C103-C130, wherein the compound is a compound according to Formula IIIA:

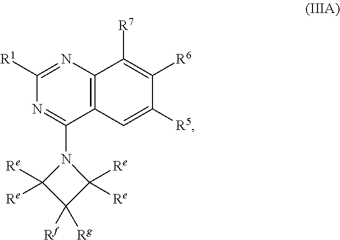

or a salt (e.g., pharmaceutically acceptable salt) thereof, wherein:
each R^{c} is independently selected from deuterium, hydrogen, —OR¹²═O, =N(R¹⁴), —C(O)(C₁₋₆ alkylene)CN, —C(O)(C₁₋₆ alkylene)OR¹², —C(O)(C₁₋₆alkyl), —C(O)N(R¹⁴)₂, —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰; and
R^{f} and R^{g} are each independently selected from hydrogen, deuterium, —OR¹², ═O, —C(O)(C₁₋₆ alkylene)CN, —C(O)(C₁₋₆ alkylene)OR¹⁴, —C(O)(C₁₋₆alkyl), —S(O)₂(C₁₋₆alkyl), —N(R¹)C(O)C₁₋₆alkyl), —N(R¹¹)C(O)(C₁₋₆ alkylene)OR¹⁴, halogen, and C₁₋₆alkyl, wherein any C₁₋₆alkyl is unsubstituted or substituted with one or more R²⁰, or
R^{f} and R^{g} join together to form a 3-6 membered carbocycle or heterocycle that is unsubstituted or substituted with one or more substituents selected from —OR¹², ═O, —C(O)(C₁₋₆ alkylene)CN, —C(O)(C₁₋₆ alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰.
C132. The compound of embodiment C131, wherein each R^{e}, R^{f}, and R^{g} is independently selected from hydrogen, —OR¹², ═O, —C(O)(C₁₋₆ alkylene)CN, —C(O)(C₁₋₆ alkylene)OR¹¹, —C(O)C₁₋₆alkyl), —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰.
C133. The compound of embodiment C131, wherein each R^{e}, R^{f}, and R^{g} is independently selected from hydrogen, —OR¹⁴, —C(O)(C₁₋₆ alkylene)CN, —C(O)(C₁₋₆ alkylene)OR¹⁴, —C(O)C₁₋₆alkyl), halogen, and C₁₋₆alkyl that is unsubstituted or substituted with one or more R²⁰.
C134. The compound of any one of embodiments C131-C133, wherein each R^{e} is hydrogen.
C135. The compound of any one of embodiments C131-C134, wherein R^{f} and R^{g} join together to form a 3-6 membered carbocycle or heterocycle that is unsubstituted or substituted with one or more substituents selected from —OR¹², ═O, —C(O)(C₁₋₆ alkylene)CN, —C(O)(C₁₋₆ alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆ alkylene)OR¹², halogen, and C₁₋₆alkyl, wherein any C₁₋₆alkyl is unsubstituted or substituted with one or more R².
C136. The compound of embodiment C135, wherein R^{f} and R^{g} join together to form a 3-6 membered carbocycle or heterocycle that is unsubstituted.
C137. The compound of embodiment C135, wherein R^{f} and R^{g} join together to form a 3-6 membered carbocycle or heterocycle that is substituted with one or more substituents selected from —OR¹², —O, —C(O)(C₁₋₆ alkylene)CN, —C(O)(C₁₋₄ alkylene)OR¹⁴, —C(O)(C₁₋₆alkyl), —S(O)₂(C₁₋₄alkyl), —N(R¹⁴)C(O)(C₁₋₆alkyl), —N(R¹⁴)C(OC₁₋₆ alkylene)OR¹⁴, halogen, and C₁₋₆alkyl, wherein any C₁₋₆alkyl is unsubstituted or substituted with one or more R²⁰.
C138. The compound of embodiment C135, wherein R^{f} and R^{g} join together to form a 3-6 membered carbocycle that is unsubstituted or substituted with one or more substituents selected from —OR¹², ═O, —C(O)(C₁₋₆ alkylene)CN, —C(O)(C₁₋₆ alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)C₁₋₆ alkylene)OR¹⁴, halogen, and C₁₋₆alkyl, wherein any C₁₋₆alkyl is unsubstituted or substituted with one or more R²⁰.
C139. The compound of embodiment C138, wherein R^{f} and R^{g} join together to form a cyclopropane that is unsubstituted or substituted with one or more substituents selected from —OR¹², ═O, —C(O)(C₁₋₆ alkylene)CN, —C(O)(C₁₋₆ alkylene)OR¹⁴, —C(O)(C₁₋₆akyl), —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰.
C140. The compound of embodiment C135, wherein R^{f} and R^{g} join together to form a 3-6 membered heterocycle that is unsubstituted or substituted with one or more substituents selected from —OR¹², ═O, —C(O)(C₁₋₆ alkylene)CN, —C(O)(C₁₋₆ alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰.
C141. The compound of embodiment C140, wherein R^{f} and R^{g} join together to form an oxetane that is unsubstituted or substituted with one or more substituents selected from —OR², ═O, —C(O)(C₁₋₆ alkylene)CN, —C(O)(C₁₋₆ alkylene)OR¹⁴, —C(O(C₁₋₆alkyl), —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰.
C142. The compound of any one of embodiments C103-C130, wherein the compound is a compound according to Formula IIIB:

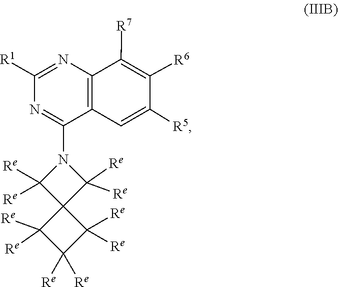

or a salt (e.g., pharmaceutically acceptable salt) thereof, wherein:
each R^{e} is independently selected from hydrogen, deuterium, —OR¹², ═O, =N(R¹⁴), —C(O)(C₁₋₆ alkylene)CN, —C(O)(C₁₋₆ alkylene)OR¹⁴, —C(O)(C₁₋₆alkyl), —C(O)N(R¹⁴)₂, —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰.
C143. The compound of embodiment C142, wherein each R′ is hydrogen.
C144. The compound of any one of embodiments C103-C130, wherein the compound is a compound according to Formula IIIC:

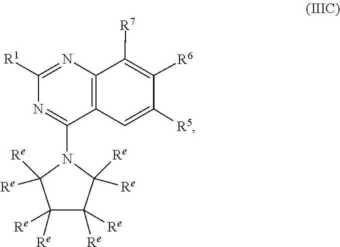

or a salt (e.g., pharmaceutically acceptable salt) thereof, wherein:
each R^{e} is independently selected from hydrogen, deuterium, —OR¹², ═O, =N(R¹⁴), —C(O)(C₁₋₆ alkylene)CN, —C(O)(C₁₋₆ alkylene)OR¹⁴, —C(O)(C₁₋₆alkyl), —C(O)N(R¹⁴)₂, —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰.
C145. The compound of embodiment C144, wherein each R^{e} is hydrogen.
C146. The compound of any one of embodiments C103-C130, wherein the compound is a compound according to Formula IIIC1:

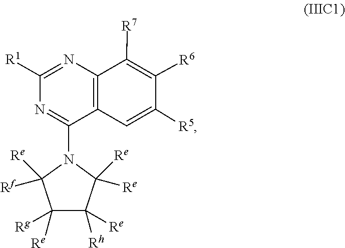

or a salt (e.g., pharmaceutically acceptable salt) thereof, wherein:
each R^{e} is independently selected from hydrogen, deuterium, —OR¹², ═O, =N(R¹⁴), —C(O)(C₁₋₆ alkylene)CN, —C(O)(C₁₋₆ alkylene)OR¹⁴, —C(O)(C₁₋₆alkyl), —C(O)N(R¹⁴)₂, —S(O)₂(C₁₋₆alkyl), —N(R¹⁴)C(O)(C₁₋₆alkyl), —N(R¹⁴)C(O)(C₁₋₆ alkylene)OR¹², halogen, and C₁₋₆alkyl, wherein any C₁₋₆alkyl is unsubstituted or substituted with one or more R²⁰; and
R^{f}, R^{g}, and R^{h} are each independently selected from hydrogen, deuterium, —OR¹², ═O, —C(O)(C₁₋₆ alkylene)CN, —C(O)(C₁₋₆ alkylene)OR¹², —C(O)(C₁₋₆alkyl), —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰, optionally wherein
(i) R^{f} and R^{g} join together to form a 3-6 membered carbocycle or heterocycle that is unsubstituted or substituted with one or more substituents selected from —OR¹², ═O, —C(O)(C₁₋₆ alkylene)CN, —C(O)(C₁₋₆ alkylene)OR¹⁴, —C(O)(C₁₋₄alkyl), —S(O)₂(C₁₋₆alkyl), —N(R¹⁴)C(O)(C₁₋₆ alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰; or
(ii) R^{g} and R^{h} join together to form a 3-6 membered carbocycle or heterocycle that is unsubstituted or substituted with one or more substituents selected from —OR¹², ═O, —C(O)C₁₋₆ alkylene)CN, —C(O)(C₁₋₆ alkylene)OR¹⁴, —C(O)(C₁₋₆alkyl), —S(O)₂(C₁₋₄alkyl), —N(R¹⁴)C(O)(C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰.
C147. The compound of embodiment C146, wherein each R^{e} is hydrogen.
C148. The compound of embodiment C146 or C147, wherein R^{f} and R^{g} join together to form a 3-6 membered carbocycle or heterocycle that is unsubstituted or substituted with one or more substituents selected from —OR², ═O, —C(O)(C₁₋₆ alkylene)CN, —C(O)(C₁₋₆ alkylene)OR¹⁴, —C(O)(C₁₋₆alkyl), —S(O)₂(C₁₋₆alkyl), —N(R¹⁴)C(O)(C₁₋₄alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰.
C149. The compound of embodiment C148, wherein R^{f} and R^{g} join together to form a 3-6 membered carbocycle that is unsubstituted or substituted with one or more substituents selected from —OR², ═O, —C(O)(C₁₋₆ alkylene)CN, —C(O)(C₁₋₆ alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰.
C150. The compound of embodiment C148, wherein R^{f} and R^{g} join together to form a 3-6 membered heterocycle that is unsubstituted or substituted with one or more substituents selected from —OR¹², ═O, —C(O)(C₁₋₆ alkylene)CN, —C(O)(C₁₋₆ alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆ alkylene)OR¹², halogen, and C₁₋₆alkyl, wherein any C₁₋₆ alkyl is unsubstituted or substituted with one or more R²⁰.
C151. The compound of embodiment C146 or C147, wherein R^{g} and R^{h} join together to form a 3-6 membered carbocycle or heterocycle that is unsubstituted or substituted with one or more substituents selected from —OR¹², ═O, —C(O)(C₁₋₆ alkylene)CN, —C(O)(C₁₋₆ alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆ alkylene)OR¹², halogen, and C₁₋₆alkyl, wherein any C₁₋₆alkyl is unsubstituted or substituted with one or more R¹³.
C152. The compound of embodiment C151, wherein R^{g} and R^{h} join together to form a 3-6 membered carbocycle that is unsubstituted or substituted with one or more substituents selected from —OR¹¹, ═O, —C(O)(C₁₋₆ alkylene)CN, —C(O)(C₁₋₆ alkylene)OR¹⁴, —C(O)(C₁₋₆akyl), —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰.
C153. The compound of embodiment C151, wherein R^{g} and R^{h} join together to form a 3-6 membered heterocycle that is unsubstituted or substituted with one or more substituents selected from —OR¹², ═O, —C(O)(C₁₋₆ alkylene)CN, —C(O)(C₁₋₆ alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰.
C154. The compound of any one of embodiments C103-C130, wherein the compound is a compound according to Formula IIID:

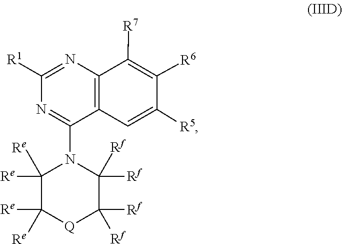

or a salt (e.g., pharmaceutically acceptable salt) thereof, wherein:
Q is selected from CR^{h}R^{j}, NR^{g}, O, S, and SO₂:
each R^{e} and R^{f} is independently selected from R¹¹ and hydrogen, wherein:
   (i) an R^{e} and an R^{f} can optionally join together to form a 4-6 membered ring;
   (ii) a first R^{f} and a second R^{f} connected to adjacent atoms can optionally join together to form a 3-5 membered ring;
   (iii) a first R^{e} and a second R^{e} connected to adjacent atoms can optionally join together to form a 3-5 membered ring; or
   (iv) a first R^{f} and a second R^{f} connected to the same atom can optionally join together to form a 3-5 membered ring,
   wherein any ring formed by one or more R^{e} and/or one or more R^{f} is unsubstituted or substituted with one or more R¹³;
R^{g}, when present, is R¹¹; and
R^{h} and R^{j}, when present, are independently selected from R¹¹ and hydrogen, or can optionally join together to form a 3-4 membered carbocycle or heterocycle that is unsubstituted or substituted with one or more substituents selected from —OR², ═O, ═N(R¹⁴), —C(O)(C₁₋₆ alkylene)CN, —C(O)(C₁₋₆ alkylene)OR¹⁴, —C(O)(C₁₋₆alkyl), —C(O)N(R¹⁴)₂, —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰.
C155. The compound of embodiment C154, wherein Q is NR^{g}.
C156. The compound of embodiment C155, wherein R^{g} is selected from —C(O)(C₁₋₆ alkylene)CN, —C(O)(C₁₋₆ alkylene)OR¹², —C(O)(C₁₋₆alkyl), —S(O)₂(C₁₋₆alkyl), and C₁₋₆alkyl, wherein any C₁₋₆alkyl is unsubstituted or substituted with one or more R²⁰.
C157. The compound of embodiment C154, wherein Q is O, S, or SO₂.
C158. The compound of embodiment C157, wherein Q is O.
C159. The compound of embodiment C154, wherein Q is CR^{h}R^{j}.
C160. The compound of embodiment C159, wherein the compound is a compound according to Formula IIIE:

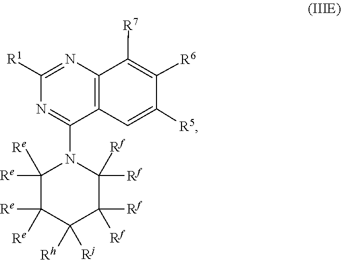

or a salt (e.g., pharmaceutically acceptable salt) thereof.
C161. The compound of embodiment C160, wherein an R^{e} and an R^{f} join together to form a 4-6 membered ring.
C162. The compound of embodiment C160 or C161, wherein R^{h} and R^{j} are independently selected from R¹¹ and hydrogen.
C163. The compound of any one of embodiments C160-C162, wherein R^{h} and/or R^{j} is selected from deuterium, —OR¹², ═O, —C(O)C₁₋₆ alkylene)CN, —C(O)(C₁₋₆ alkylene)OR¹⁴, —C(O)(C₁₋₄alkyl), —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰.
C164. The compound of any one of embodiments C160-C162, wherein R^{e} and R^{f} are each hydrogen.
C165. The compound of embodiment C160, wherein R^{h} and R^{j} join together to form a 3-4 membered carbocycle or heterocycle.
C1-66. The compound of embodiment C165, wherein R^{h} and R^{j} join together to form a 3-4 membered carbocycle or heterocycle that is unsubstituted.
C1-67. The compound of embodiment C165, wherein R^{h} and R^{j} join together to form a 3-4 membered carbocycle or heterocycle that is substituted with one or more substituents selected from —OR¹², ═O, —C(O)(C₁₋₆ alkylene)CN, —C(O)(C₁₋₆ alkylene)OR¹⁴, —C(O)(C₁₋₆akyl), —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆alkyl that is unsubstituted or substituted with one or more R²⁰.
C168. The compound of any one of embodiments C165-C167, wherein no combination of one or more R^{e}s and/or one or more R^{f}s join together to form a ring.
C169. The compound of any one of embodiments C165-C168, wherein each R^{e} and R^{f} is hydrogen.
C170. The compound of embodiment C154, wherein the compound is a compound according to Formula IIIF, IIIG, or IIIH:

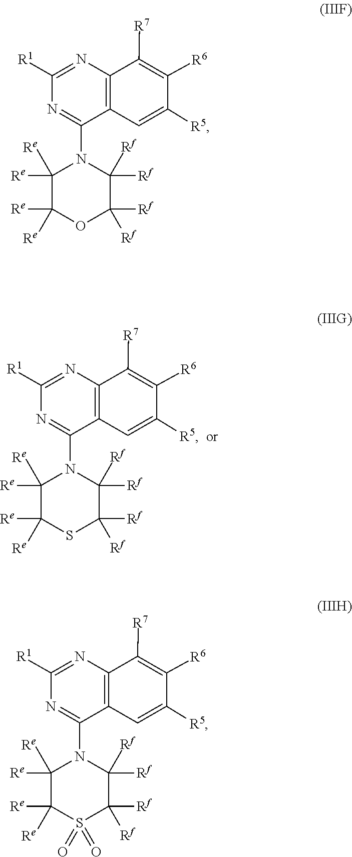

or a salt (e.g., pharmaceutically acceptable salt) thereof.
C171. The compound of embodiment C170, wherein the compound is a compound of Formula IIIF or a salt (e.g., a pharmaceutically acceptable salt) thereof.
C172. The compound of embodiment C170, wherein the compound is a compound of Formula IIIG or a salt (e.g., a pharmaceutically acceptable salt) thereof.
C173. The compound of embodiment C170, wherein the compound is a compound of Formula IIIH or a salt (e.g., a pharmaceutically acceptable salt) thereof.
C174. The compound of any one of embodiments C170-C173, wherein each R^{e} and R^{f} is independently selected from R¹¹ and hydrogen.
C175. The compound of any one of embodiments C170-C173, wherein an R^{e} and an R^{f} join together to form a 4-6 membered ring.
C176. The compound of embodiment C154, wherein the compound is a compound according to Formula IIU:

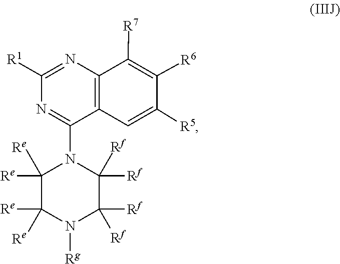

or a salt (e.g., pharmaceutically acceptable salt) thereof.
C177. The compound of embodiment C176, wherein each R^{e} and R^{f} is independently selected from R¹¹ and hydrogen.
C178. The compound of embodiment C176, wherein an R^{e} and an R^{f} join together to form a 4-6 membered ring.
C179. The compound of any one of embodiments C154-C178, wherein the compound is a compound according to Formula IIIK, IIII, HIM, IIIN, HIP, IIIQ, or IIIR.

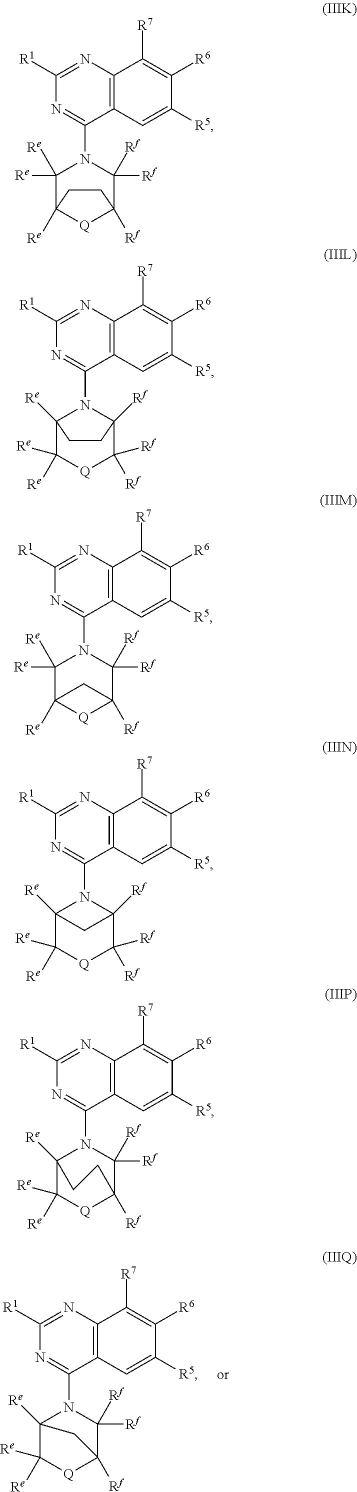

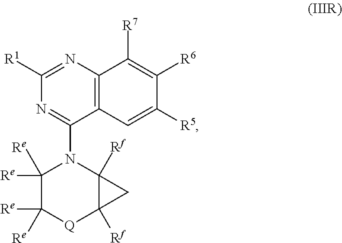

or a salt (e.g., pharmaceutically acceptable salt) wherein:
each R^{e} and R^{f} is independently selected from R¹¹ and hydrogen;
R², when present, is R¹¹, and
R^{h} and R^{j}, when present, are each independently selected from R¹¹ and hydrogen.
C180. The compound of embodiment C179, wherein the compound is a compound of Formula IIIK or a salt (e.g., a pharmaceutically acceptable salt) thereof.
C181. The compound of embodiment C179, wherein the compound is a compound of Formula IIIL or a salt (e.g., a pharmaceutically acceptable salt) thereof.
C182. The compound of embodiment C179, wherein the compound is a compound of Formula HIM or a salt (e.g., a pharmaceutically acceptable salt) thereof.
C183. The compound of embodiment C179, wherein the compound is a compound of Formula IIIN or a salt (e.g., a pharmaceutically acceptable salt) thereof.
C184. The compound of embodiment C179, wherein the compound is a compound of Formula HIP or a salt (e.g., a pharmaceutically acceptable salt) thereof.
C185. The compound of embodiment C179, wherein the compound is a compound of Formula IIIQ or a salt (e.g., a pharmaceutically acceptable salt) thereof.
C186. The compound of embodiment C179, wherein the compound is a compound of Formula IIIR or a salt (e.g., a pharmaceutically acceptable salt) thereof.
C187. The compound of any one of embodiments C179-C186, wherein Q is selected from CR^{h}R^{j}, NR^{j}, and O.
C188. The compound of embodiment C187, wherein Q is CR^{h}R^{j}.
C189. The compound of embodiment C188, wherein R^{h} and R^{j}, when present, are each independently selected from R¹¹ and hydrogen.
C190. The compound of embodiment C189, wherein R^{e} and/or R^{j}, when present, is each independently selected from —OR¹², ═O, =N(R¹⁴), —C(O)(C₁₋₆ alkylene)CN, —C(O)(C₁₋₄ alkylene)OR¹⁴, —C(O)(C₁₋₆alkyl), —C(O)N(R¹⁴)₂, —S(O)₂(C₁₋₄alkyl), —N(R¹⁴)C(O)(C₁₋₆akyl), —N(R¹⁴)C(O)(C₁₋₆akylene)OR¹⁴ halogen, and C₁₋₆alkyl, wherein any C₁₋₆alkyl is unsubstituted or substituted with one or more R²⁰.
C191. The compound of embodiment C189, wherein R^{e} and R^{j} are each hydrogen.
C192. The compound of embodiment C187, wherein Q is NR^{g}.
C193. The compound of embodiment C192, wherein R¹ is selected from —C(O)(C₁₋₆ alkylene)CN, —C(O)(C₁₋₆ alkylene)OR¹⁴, —C(O)(C₁₋₆alkyl), —C(O)N(R¹⁴)₂, and C₁₋₆akyl, wherein any C₁₋₆alkyl is unsubstituted or substituted with one or more R²⁰.
C194. The compound of embodiment C187, wherein Q is O.
C195. The compound of any one of embodiments C179-C186, wherein Q is S or SO₂.
C196. The compound of any one of embodiments C179-C195, wherein each R^{e} and R^{f} is independently selected from R¹¹ and hydrogen.
C197. The compound of embodiment C196, wherein R^{e} and/or R^{f} is each independently selected from deuterium, —OR¹², —O, —N(R¹⁴), —C(O)(C₁₋₆ alkylene)CN, —C(O)(C₁₋₆ alkylene)OR¹⁴, —C(O)(C₁₋₆alkyl), —C(O)N(R¹⁴)₂, —S(O)₂(C₁₋₆alkyl), —N(R¹⁴)C(O)(C₁₋₆akyl), —N(R¹⁴)C(O)(C₁₋₆ alkylene)OR¹⁴, halogen, and C₁₋₆alkyl that is unsubstituted or substituted with one or more R²⁰.
C198. The compound of embodiment C197, wherein each Rⁱ and R^{j} is hydrogen.
C199. The compound of any one of embodiments C103-C130, wherein the compound is a compound according to Formula IIIS:

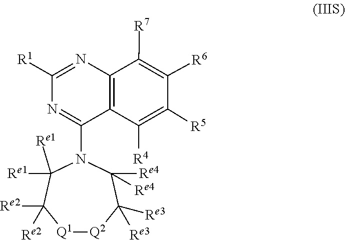

or a salt (e.g., pharmaceutically acceptable salt) wherein:
Q¹ is selected from NR^{g1}, O, SR^{h}₂, and CRⁱR^{j};
Q² is selected from NR^{g2}, O, SR^{h}₂, and CRⁱR^{j};
R^{g1} and R^{g2}, when present, are independently selected from hydrogen, —C(O)(C₁₋₆alkyl), and C₁₋₆ alkyl, wherein any C₁₋₄alkyl is unsubstituted or substituted with one or more R²⁰;
each R^{h} is absent or, when present, is independently selected from ═O, =N(R¹⁴), and C₁₋₆alkyl, wherein any C₁₋₆alkyl is unsubstituted or substituted with one or more R²⁰;
each Rⁱ and R^{j}, when present, are independently selected from hydrogen, deuterium, —OR¹², ═O, =N(R¹⁴), —C(O)(C₁₋₆ alkylene)CN, —C(O)(C₁₋₆ alkylene)OR¹⁴, —C(O)(C₁₋₆alkyl), —C(O)N(R¹⁴)₂, —S(O)₂(C₁₋₆alkyl), —N(R¹⁴)C(O)(C₁₋₆alkyl), —N(R¹⁴)C(O)(C₁₋₆ alkylene)OR¹², halogen, and C₁₋₆alkyl, wherein any C₁₋₆alkyl is unsubstituted or substituted with one or more R²¹; and
each R^{e1}, R^{e2}, R^{e3}, and R^{e4} are independently selected from hydrogen, deuterium, —OR¹², ═O, =N(R¹⁴), —C(O)C₁₋₆ alkylene)CN, —C(O)(C₁₋₆ alkylene)OR¹², —C(O)(C₁₋₆alkyl), —C(O)N(R¹⁴)₂, —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰,
wherein
   (i) an R^{e2} and an R^{e3} optionally join together to form a 5-6 membered ring; or
   (ii) when Q² is NR^{g2}, R^{g2} and an R^{e3}, together with the atoms to which they are attached, optionally join together to form a 5-membered heterocycle or heteroaryl that is unsubstituted or substituted with one or more R¹¹, wherein each R¹¹ is independently selected from deuterium, —OR¹², —O, —N(R¹⁴), —C(O)(C₁₋₆ alkylene)CN, —C(O)(C₁₋₆ alkylene)OR¹⁴, —C(O)(C₁₋₆alkyl), —C(O)N(R¹⁴)₂, —S(O)₂(C₁₋₆alkyl), —N(R¹⁴)C(O)((C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰.
C200. The compound of embodiment C199, wherein Q¹ and Q² are each CRⁱR^{j}.
C201. The compound of embodiment C200, wherein at least one R^{e1}, R^{e2}, R^{e3}, or R^{e4} is selected from —OR¹², ═O, =N(R¹⁴), —C(O)(C₁₋₆ alkylene)CN, —C(O)(C₁₋₆ alkylene)OR¹⁴, —C(O)(C₁₋₆alkyl). —C(O)N(R¹⁴)₂, —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰.
C202. The compound of embodiment C201, wherein each R^{e1}, R^{e2}, R^{e3}, and R^{e4} is hydrogen.
C203. The compound of embodiment C200, wherein an R^{e2} and an R^{e3} join together to form a 5-6 membered ring.
C204. The compound of embodiment C199, wherein Q¹ is CRⁱR^{j} and Q² is NR^{g2}.
C205. The compound of embodiment C204, wherein R^{g2} and an R^{e3}, together with the atoms to which they are attached, join together to form a 5-membered heterocycle or heteroaryl that is unsubstituted or substituted with one or more R¹¹.
C206. The compound of embodiment C204, wherein R^{g2} is selected from hydrogen, —C(O)(C₁₋₆alkyl), and C₁₋₆alkyl, wherein any C₁₋₆alkyl is unsubstituted or substituted with one or more R²⁰.
C207. The compound of embodiment C199, wherein Q² is CRⁱR^{j} and Q¹ is NR^{g1}.
C208. The compound of embodiment C207, wherein R^{g1} is selected from hydrogen, —C(O)(C₁₋₆alkyl), and C₁₋₆alkyl, wherein any C₁₋₆alkyl is unsubstituted or substituted with one or more R²⁰.
C209. The compound of embodiment C207 or C208, wherein each R^{e1}, R^{e2}, R^{e3}, and R⁴ is hydrogen.
C210. The compound of embodiment C199, wherein (i) Q¹ is CRⁱR^{j} and Q² is O or (ii) Q² is CRⁱR^{j} and Q¹ is O.
C211. The compound of embodiment C210, wherein each R^{e1}, R^{e2}, R^{e3}, and R^{e4} is hydrogen.
C212. The compound of embodiment C210, wherein an R^{e2} and an R^{e3} join together to form a 5-6 membered ring.
C213. The compound of embodiment C199, wherein (i) Q¹ is CRⁱR^{j} and Q² is SR^{h}₂ or (ii) Q² is CRⁱR^{j} and Q¹ is SR^{h}₂.
C214. The compound of embodiment C213, wherein each R^{e1}, R^{e2}, R^{e3}, and R^{e4} is hydrogen.
C215. The compound of embodiment C213, wherein an R^{e2} and an R^{e3} join together to form a 5-6 membered ring.
C216. The compound of any one of embodiments C213-C215, wherein each R^{h} is ═O.
C217. The compound of any one of embodiments C200-C216, wherein each Rⁱ and R^{j} are independently selected from hydrogen, deuterium. —OR², ═O, =N(R¹⁴), —C(O(C₁₋₆ alkylene)CN, —C(O)C₁₋₆ alkylene)OR¹⁴, —C(O)(C₁₋₆alkyl), —C(O)N(R¹⁴)₂, —S(O)₂(C₁₋₆alkyl), —N(R¹⁴)C(O)(C₁₋₆alkyl), —N(R¹⁴)C(O)(C₁₋₆ alkylene)OR¹⁴, halogen, and C₁₋₆alkyl, wherein any C₁₋₆alkyl is unsubstituted or substituted with one or more R²⁰.
C218. The compound of embodiment C217, wherein each Rⁱ and R^{j} are hydrogen.
C219. The compound of embodiment C199, wherein (i) NR^{g1} and Q² is SR^{h}₂ or (ii) NR^{g2} and Q¹ is SR^{h}₂.
C220. The compound of embodiment C219, wherein each R^{e1}, R^{e2}, R^{e3}, and R^{e4} is hydrogen.
C221. The compound of embodiment C219, wherein an R^{e2} and an R^{e3} join together to form a 5-6 membered ring.
C222. The compound of any one of embodiments C219-C221, wherein each R^{h} is ═O.
C223. The compound of any one of embodiments C103-C130, wherein the compound is a compound according to Formula IIT:

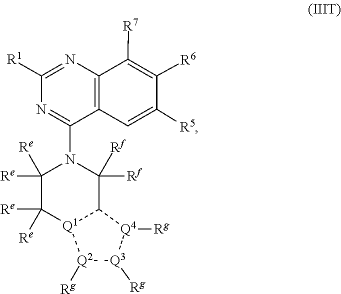

or a salt (e.g., pharmaceutically acceptable salt) wherein:
dashed lines represent single or double bonds, such that the ring containing Q¹, Q², Q³, and Q⁴ is aromatic:
Q¹, Q², Q³, and Q⁴ are independently selected from C, N, O, and S wherein at least one of Q¹, Q², Q³, and Q⁴ is N;
each R^{e} and R^{f} is independently selected from R¹¹ and hydrogen; and
each R^{g} is absent or is independently selected from R¹¹ and hydrogen.
C224. The compound of embodiment C223, wherein Q′ is C.
C225. The compound of embodiment C224, wherein Q² is C, Q³ is N, and Q⁴ is N.
C226. The compound of embodiment C223, wherein Q¹ is N.
C227. The compound of embodiment C226, wherein Q² is N, and Q³ and Q⁴ are C.
C228. The compound of embodiment C226, wherein Q² and Q³ are N, and Q⁴ is C.
C229. The compound of embodiment C226, wherein Q³ and Q⁴ are N, and Q² is C.
C230. The compound of embodiment C226, wherein Q² and Q⁴ are N, and Q³ is C.
C231. The compound of embodiment C223, wherein,

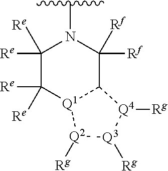

has a structure selected from

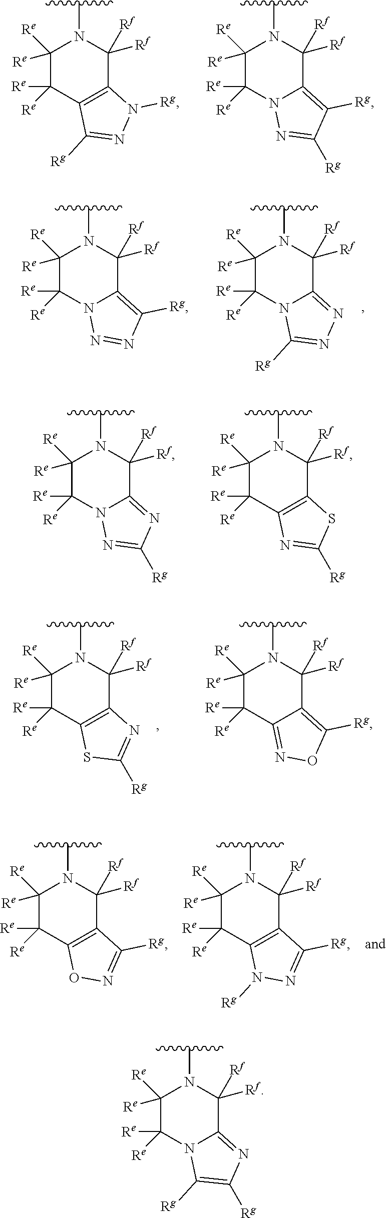

C232. The compound of any one of embodiments C223-C231, wherein each R^{g} is hydrogen or is absent.
C233. The compound ofany one of embodiments C103-C232, wherein R⁶ is a bicyclic heteroaryl that is substituted with one or more R¹³.
C234. The compound of embodiment C233, wherein R⁶ is selected from:

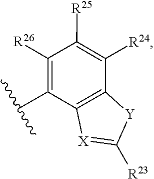

wherein:
   X is selected from N and C—CN;
   Y is selected from O and S;
   R¹¹ is selected from —N(R¹⁴)₂, C₁₋₆alkyl, and C₁₋₆alkyl-N(R¹⁴)₂, wherein any C₁₋₆akyl is unsubstituted or substituted with one or more R¹³; and
   R²⁴, R²⁵, and R²⁶ are independently selected from H, deuterium, halogen, and C₁₋₆akyl, wherein any C₁₋₆alkyl is unsubstituted or substituted with one or more R¹³.
C235. The compound of embodiment C233 or C234, wherein R¹ is selected from:

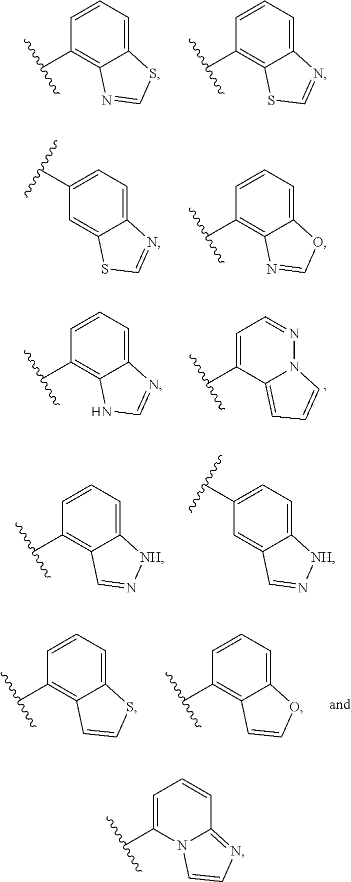

any of which is substituted with one or more R¹⁵.
C236. The compound of any one of embodiments C233-C235, wherein R^{c} is selected from:

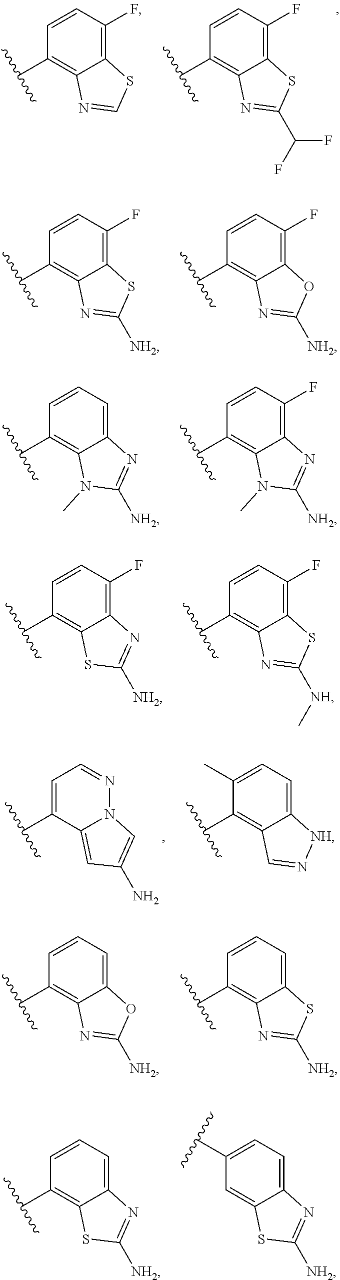

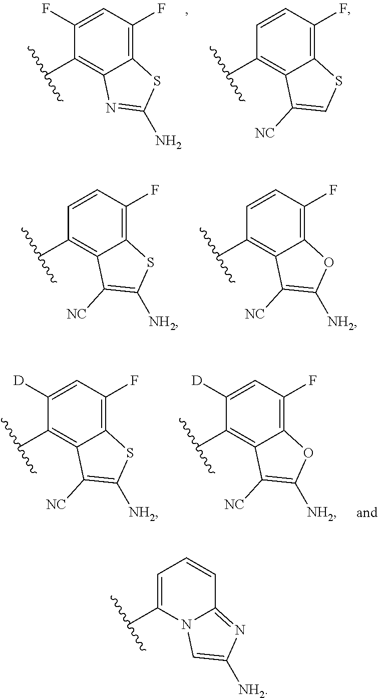

C237. The compound of any one of embodiments C233-C236, wherein R⁶ is selected from:

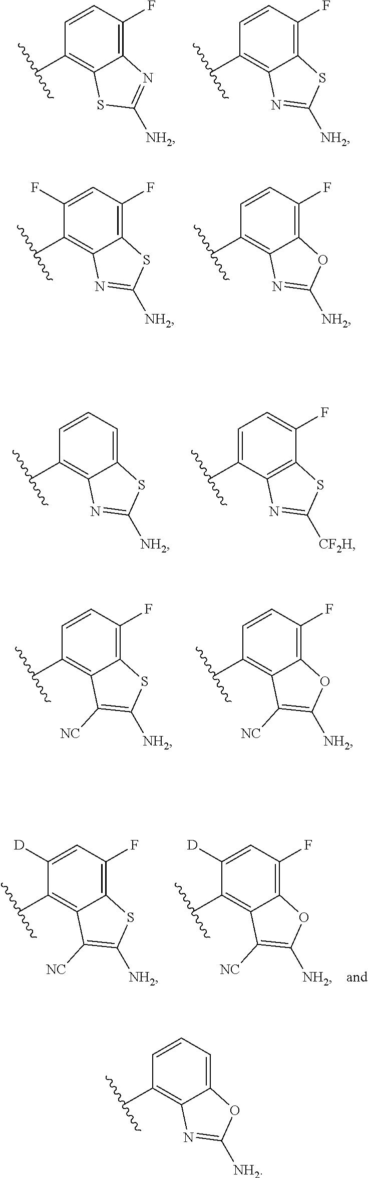

C238. The compound ofany one of embodiments C103-C232, wherein R¹ is a monocyclic heteroaryl that is substituted with one or more R²⁰.
C239. The compound of embodiment C238, wherein R⁶ is a pyridine substituted with one or more R¹³.
C240. The compound of embodiment C239, wherein R′ has the structure

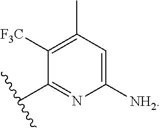 [SMILES: *c1nc(N)cc(C)c1C(F)(F)F]

C241. The compound of any one of embodiments C103-C240, wherein R¹ is selected from —OR^{g}.
C242. The compound of embodiment C241, wherein R¹ is selected from:

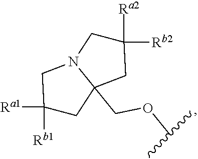

wherein R^{a1}, R^{a2}, R^{b1}, and R^{b2} are each independently selected from deuterium, halogen, C₁₋₆alkyl, —OR¹², and H, wherein R^{a2} and R^{b2} can optionally join together to form a 3-6 membered carbocycle, and wherein any C₁₋₆alkyl or 3-6 membered carbocycle is unsubstituted or is substituted with one or more R¹³.
C243. The compound of embodiment C242, wherein R¹ is selected from:

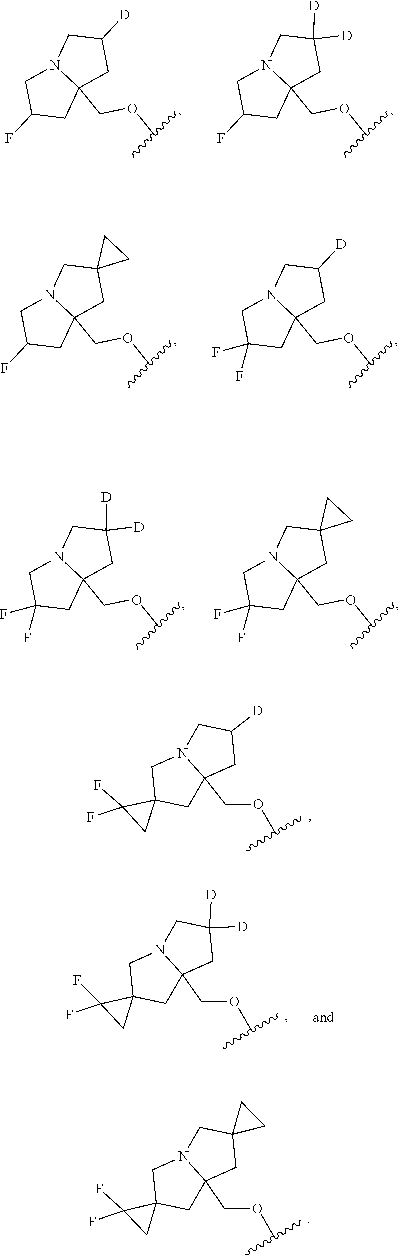

C244. The compound of embodiment C241, wherein R¹ is selected from:

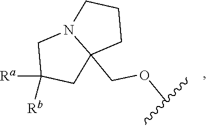

wherein R^{a} and R^{b} are each independently selected from halogen, C₁₋₆alkyl, —OR¹², and H, wherein any C₁₋₆alkyl is unsubstituted or is substituted with one or more R¹³.
C245. The compound of embodiment C244, wherein R¹ is selected from:

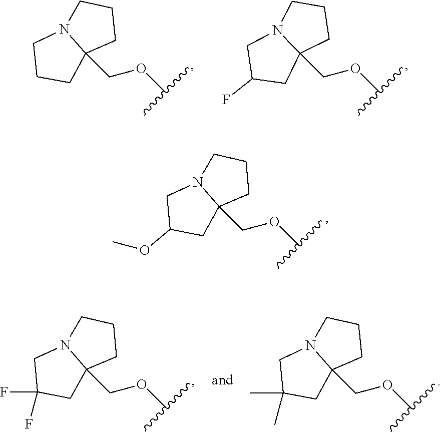 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

C246. The compound of embodiment C241, wherein R¹ is selected from:

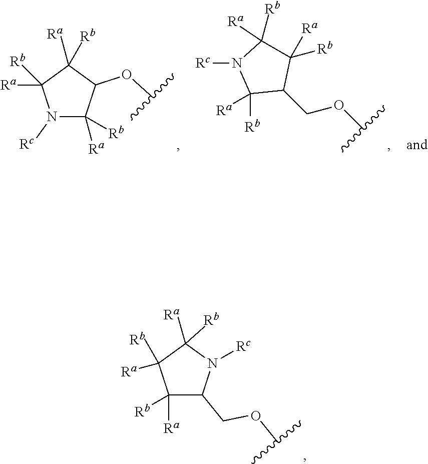

wherein each R^{a} and R^{b} is independently selected from halogen, C₁₋₆ alkyl, —OR², and H; and R^{c} is selected from C₁₋₆ alkyl, wherein an R^{a} and R^{b} or R^{c} optionally join together to form a 3-6 membered carbocycle or heterocyclic, and wherein any C₁₋₆ alkyl or 3-6 membered carbocycle or heterocycle is unsubstituted or is substituted with one or more R¹³.
C247. The compound of embodiment C246, wherein R¹ is selected from:

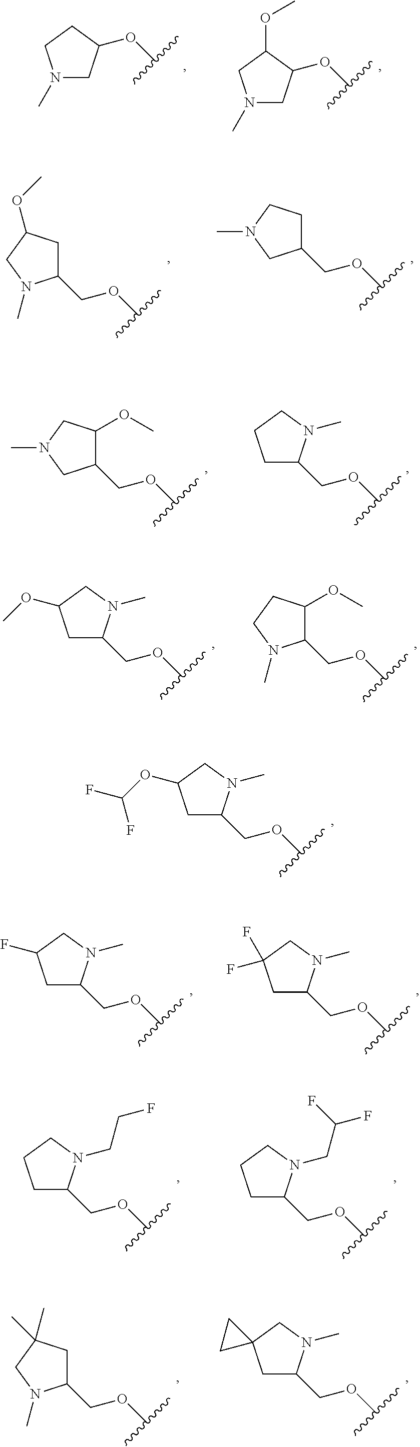

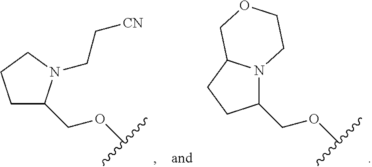 [SMILES: *OCC1CCCN1CCC#N ; *OCC1CCC2COCCN12]

C248. The compound of any one of embodiments C103-C240, wherein R¹ is selected from

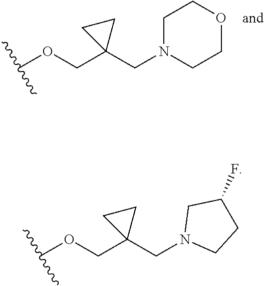 [SMILES: *OCC1(CN2CCOCC2)CC1 ; *OCC1(CN2CC[C@@H](F)C2)CC1]

C249. The compound of any one of embodiments C103-C248, wherein each R¹¹ is independently selected from —OR¹², ═O, —C(O)(C₁₋₆ alkylene)CN, —C(O)(C₁₋₆ alkylene)OR¹⁴, —C(O)(C₁₋₆alkyl), —S(O)₂(C₁₋₆alkyl), —N(R¹⁰)C(O)(C₁₋₆alkyl), —N(R¹⁴)C(O)(C₁₋₆ alkylene)OR¹², halogen, and C₁₋₆alkyl that is unsubstituted or substituted with one or more R²⁰.
C250. The compound of any one of embodiments C103-C249, wherein R¹ is a halogen (e.g., F or Cl).
C251. The compound of any one of embodiments C103-C249, wherein R¹ is selected from C₁₋₆alkyl that is unsubstituted or substituted with one or more R¹³.
C252. The compound of embodiment C251, wherein R¹ is selected from C₁₋₆alkyl that is substituted with one or more halogens or —CN.
C253. The compound of embodiment C252, wherein R¹ is selected from —CF₂H, —CF₃, —CH₂CN, and —CH₂CH₃.
C254. The compound of embodiment C253, wherein R¹ is —CF₃.
C255. The compound of any one of embodiments C103-C254, wherein the compound is a not a compound included in Table 2.
C256. A compound represented by Formula II:

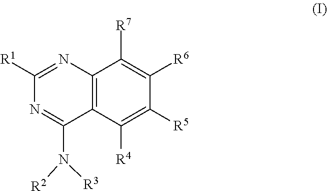

or a salt (e.g., pharmaceutically acceptable salt) thereof, wherein:
R¹ is selected from —OR⁸ and

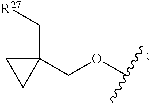

R² is selected from H and C₁₋₆alkyl, wherein any C₁₋₆ alkyl is unsubstituted or is substituted with one or more R¹³;
R³ is selected from a 3-11 membered carbocycle that is unsubstituted or substituted with one or more R¹⁰;
R⁴ is H:
R⁵ is selected from H, halogen, —CN, —OR², a 3-6 membered heterocycle, a 5-6 membered heteroaryl, and C₁₋₆alkyl, wherein any C₁₋₆alkyl is unsubstituted or substituted with one or more R¹³;
R⁶ is a bicyclic heteroaryl substituted with one or more R¹⁵;
R⁷ is selected from halogen, —CN, and H;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R¹⁰ is independently selected from —OR¹², —C(O)(C₁₋₆ alkylene)CN, —C(O)(C₁₋₆ alkylene)OH, —C(O)(C₁₋₆alkyl), —C(O)N(R¹⁴)₂, —S(O)₂(C₁₋₆alkyl), halogen, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹³;
each R² is independently selected from —OH, —OC₁₋₆alkyl, —CN, —NH₂, —NHC₁₋₆alkyl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen; and
R^{a} and R^{b} are each independently selected from deuterium, halogen, C₁₋₆ alkyl, a 3-6 membered carbocycle, —OR¹², and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³.
C257. The compound of embodiment C256, wherein R³ is a 3-6 membered carbocycle that is unsubstituted or is substituted with one or more R¹⁰.
C258. The compound of embodiment C257, wherein R³ is a 3-6 membered carbocycle that is substituted with 1-4 R¹⁰.
C259. The compound of embodiment C257, wherein R³ is a cyclopropane that is substituted with 0-4 R¹⁰.
C260. The compound of embodiment C257, wherein R³ is a cyclobutane that is substituted with 0-4 R¹⁰.
C261. The compound of embodiment C257, wherein R³ is a cyclopentane that is substituted with 0-4 R¹⁰.
C262. The compound of embodiment C257, wherein R³ is a cyclohexane that is substituted with 0-4 R¹⁰.
C263. The compound of any one of embodiments C256-C258, wherein R³ is a spirocycle that is unsubstituted or is substituted with one or more R²⁰.
C264. The compound of any one of embodiments C256-C258, wherein R³ is a bridged carbocycle that is unsubstituted or is substituted with one or more R¹⁰.
C265. The compound of any one of embodiments C256-C264, wherein R³ is substituted with one or more R¹⁰, wherein at least one R¹⁰ is selected from —OR¹² and a C₁₋₆alkyl substituted with —OH.
C266. The compound of any one of embodiments C256-C265, wherein R³ is substituted with one or more R¹⁰, wherein at least one R¹⁰ is an unsubstituted C₁₋₆alkyl.
C267. The compound of any one of embodiments C256-C266, wherein the compound is a compound according to Formula IA, IB. IC, ID, IE, or IF:

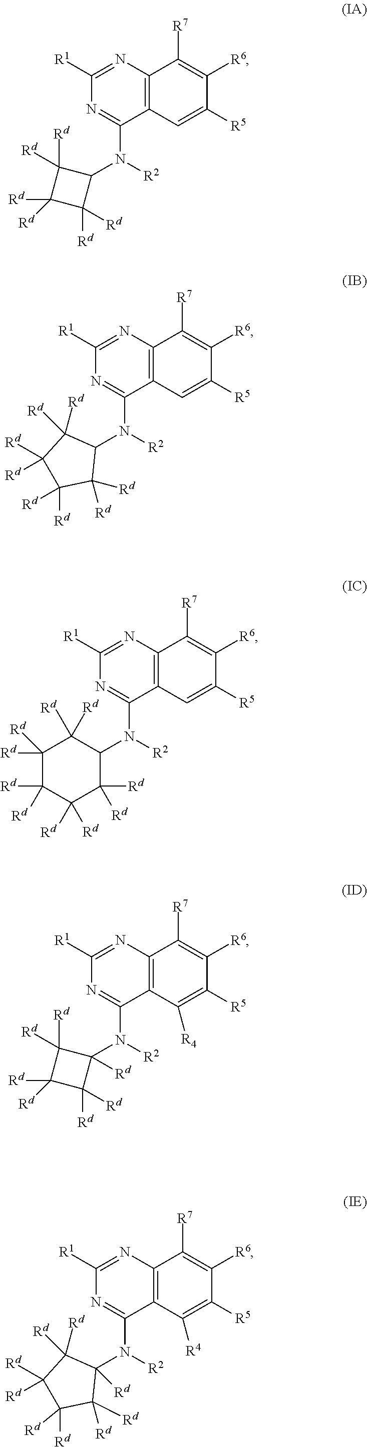

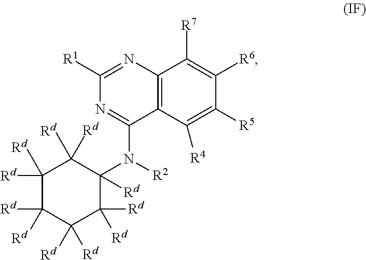

or a salt (e.g., pharmaceutically acceptable salt) thereof, wherein:
each R^{d} is independently selected from H, —OR², —C(O)(C₁₋₆ alkylene)CN, —C(O)(Cr, alkylene)OH, —C(O)(C₁₋₆alkyl), —C(O)N(R¹⁴)₂, —S(O)₂(C₁₋₆alkyl), halogen, and C₁₋₆alkyl, wherein any C₁₋₆alkyl is unsubstituted or substituted with one or more R²⁰.
C268. The compound of embodiment C267, wherein the compound is a compound according to Formula IA, or a salt (e.g., pharmaceutically acceptable salt) thereof.
C269. The compound of embodiment C267, wherein the compound is a compound according to Formula IB, or a salt (e.g., pharmaceutically acceptable salt) thereof.
C270. The compound of embodiment C267, wherein the compound is a compound according to Formula IC, or a salt (e.g., pharmaceutically acceptable salt) thereof.
C271. The compound of embodiment C267, wherein the compound is a compound according to Formula ID, or a salt (e.g., pharmaceutically acceptable salt) thereof.
C272. The compound of embodiment C267, wherein the compound is a compound according to Formula IE, or a salt (e.g., pharmaceutically acceptable salt) thereof.
C273. The compound of embodiment C267, wherein the compound is a compound according to Formula IF, or a salt (e.g., pharmaceutically acceptable salt) thereof.
C274. The compound of any one of embodiments C267-C273, wherein at least one R^{d} is selected from —OR¹², —C(O)(C₁₋₆ alkylene)CN, —C(O)(C₁₋₆ alkylene)OH, —C(O)(C₁₋₆alkyl), —C(O)N(R¹⁴)₂, —S(O)₂(C₁₋₆alkyl), halogen, and C₁₋₆alkyl, wherein any C₁₋₆alkyl is unsubstituted or substituted with one or more R².
C275. The compound of embodiment C274, wherein at least one R^{d} is selected from —OR² and C₁₋₆alkyl, wherein any C₁₋₆alkyl is unsubstituted or substituted with one or more R²⁰.
C276. The compound of any one of embodiments C256-C275, wherein R¹ is selected from:

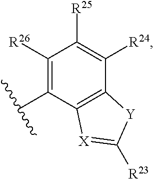

wherein:
   X is selected from N and C—CN;
   Y is selected from O and S;
   R²³ is selected from —N(R¹⁴)₂, C₁₋₆alkyl, and C₁₋₆alkyl-N(R¹⁴)₂, wherein any C₁₋₆alkyl is unsubstituted or substituted with one or more R¹³; and
   R²⁴, R^{2S}, and R²⁶ are independently selected from H, deuterium, halogen, and C₁₋₆alkyl, wherein any C₁₋₆alkyl is unsubstituted or substituted with one or more R¹³.
C277. The compound of any one of embodiments C256-C276, wherein R⁶ is selected from:

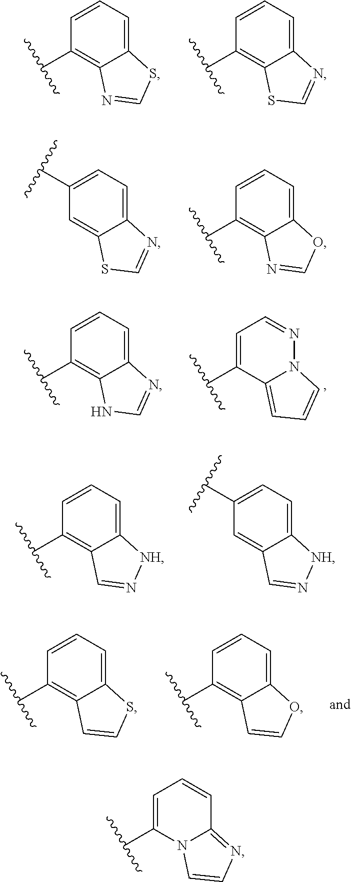

any of which is substituted with one or more R¹³.
C278. The compound of any one of embodiments C256-C277, wherein R¹ is selected from:

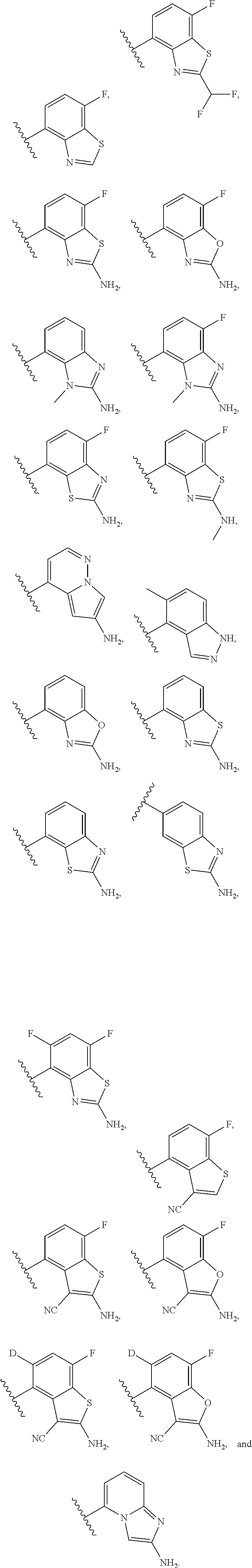

C279. The compound of any one of embodiments C256-C278, wherein R⁶ is selected from:

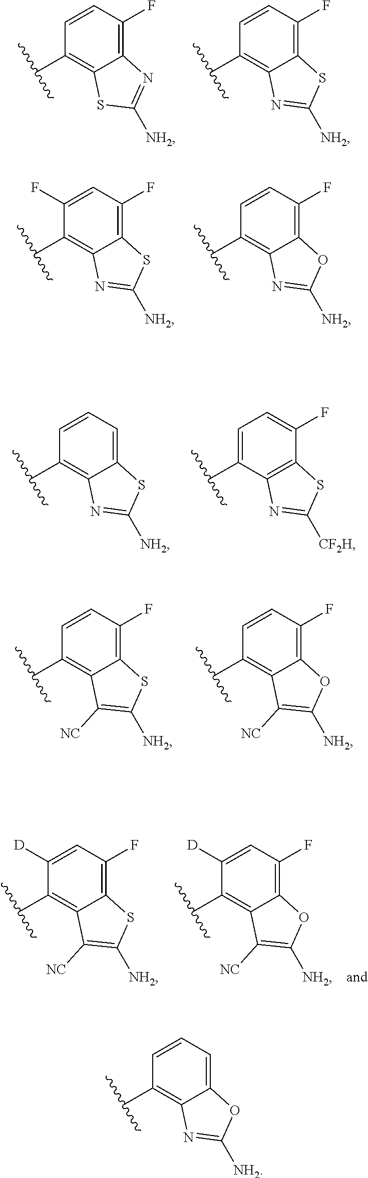

C280. The compound of any one of embodiments C256-C279, wherein the compound is a compound according to Formula IA1, IB1, IC1, ID1, IE1, or IF1:

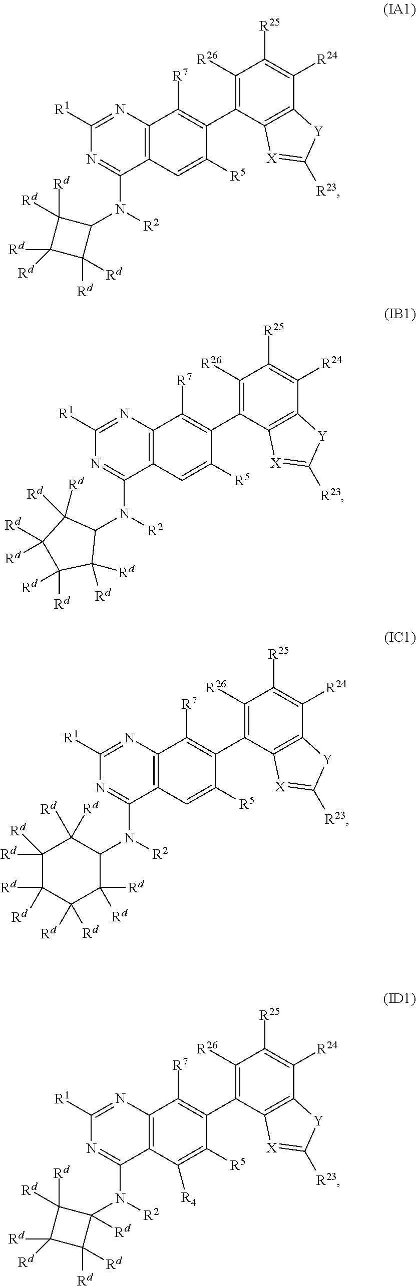

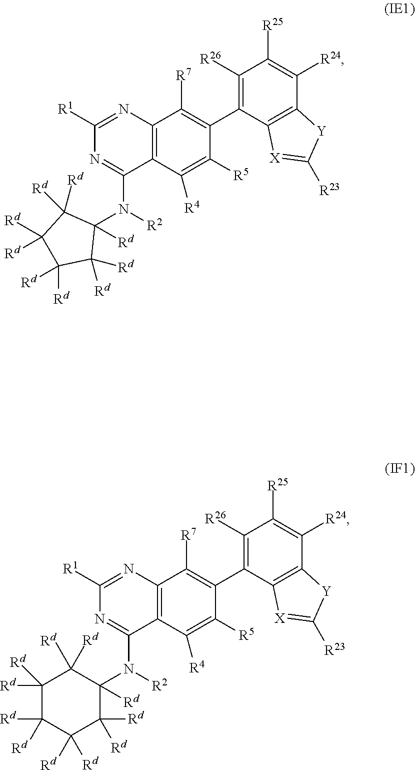

or a salt (e.g., pharmaceutically acceptable salt) thereof, wherein:
each R^{d} is independently selected from H, —OR¹², —C(O)(C₁₋₆ alkylene)CN, —C(O)C₁₋₆ alkylene)OH, —C(O)(C₁₋₆akyl), —C(O)N(R¹⁴)₂, —S(O)₂(C₁₋₆alkyl), halogen, and C₁₋₆alkyl, wherein any C₁₋₆alkyl is unsubstituted or substituted with one or more R²⁰;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆akyl is unsubstituted or substituted with one or more R¹³; and
R²⁴, R²⁵, and R²⁶ are independently selected from H, deuterium, halogen, and C₁₋₆alkyl, wherein any C₁₋₆alkyl is unsubstituted or substituted with one or more R¹³.
C281. The compound of embodiment C280, wherein the compound is a compound according to Formula IA1, or a salt (e.g., pharmaceutically acceptable salt) thereof.
C282. The compound of embodiment C280, wherein the compound is a compound according to Formula IB1, or a salt (e.g., pharmaceutically acceptable salt) thereof.
C283. The compound of embodiment C280, wherein the compound is a compound according to Formula IC1, or a salt (e.g., pharmaceutically acceptable salt) thereof.
C284. The compound of embodiment C280, wherein the compound is a compound according to Formula ID1, or a salt (e.g., pharmaceutically acceptable salt) thereof.
C285. The compound of embodiment C280, wherein the compound is a compound according to Formula IE1, or a salt (e.g., pharmaceutically acceptable salt) thereof.
C286. The compound of embodiment C280, wherein the compound is a compound according to Formula IF1, or a salt (e.g., pharmaceutically acceptable salt) thereof.
C287. The compound of any one of embodiments C280-C286, wherein X is C—CN, Y is S, and R²³ is —N(R¹²)₂.
C288. The compound of any one of embodiments C280-C287, wherein one or more of R²⁴, R²⁵, and R²⁶ is a halogen (e.g., F).
C289. The compound of any one of embodiments C256-C288, wherein R¹ is selected from —OR⁸.
C290. The compound of embodiment C289, wherein R¹ is selected from:

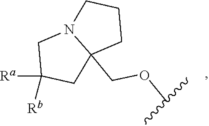

wherein R^{a} and R^{b} are each independently selected from halogen, C₁₋₆alkyl, —OR², and H, wherein any C₁₋₆alkyl is unsubstituted or is substituted with one or more R¹³.
C291. The compound of embodiment C290, wherein R¹ is selected from:

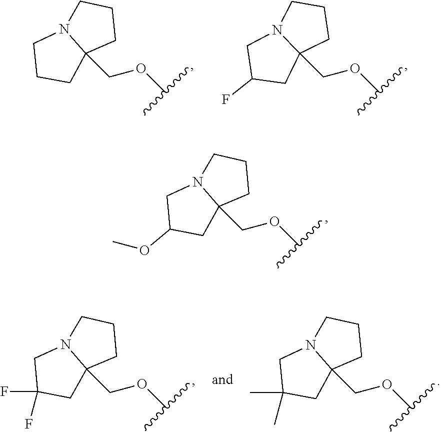 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

C292. The compound of embodiment C289, wherein R¹ is selected from:

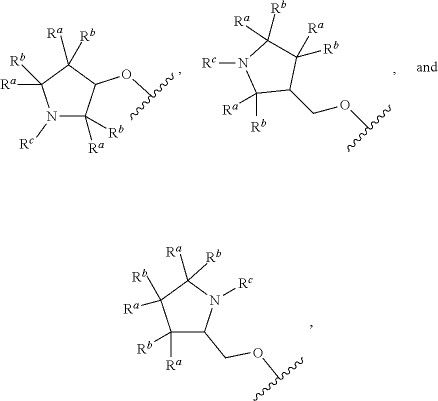

wherein each R^{a} and R^{b} is independently selected from halogen, C₁₋₆ alkyl, —OR¹², and H; and R^{c} is selected from C₁₋₆ alkyl, wherein any C₁₋₆ alkyl is unsubstituted or is substituted with one or more R¹³, and wherein an R^{a} and R^{b} or R optionally join together to form a 3-6 membered carbocycle or heterocycle.

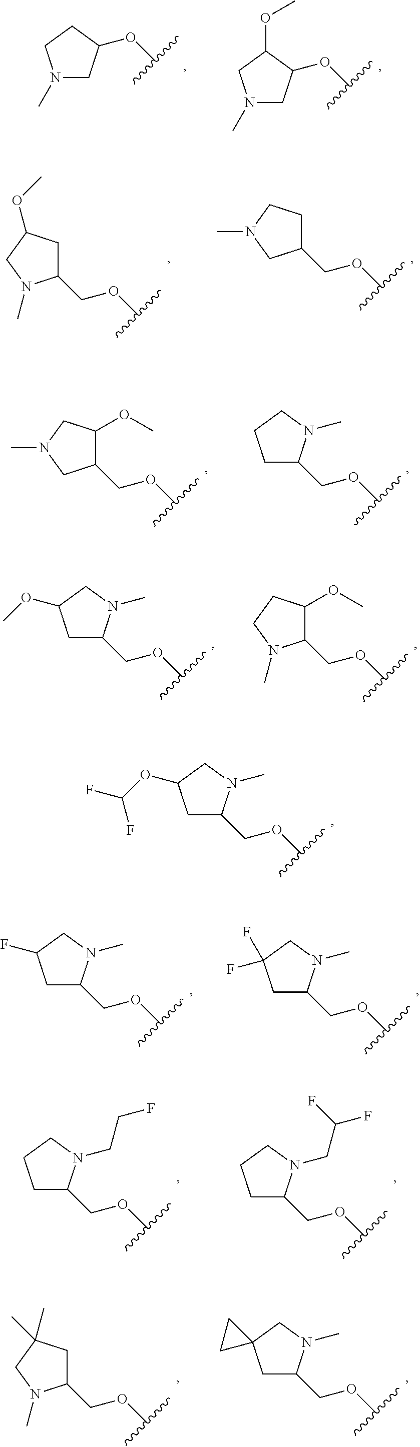

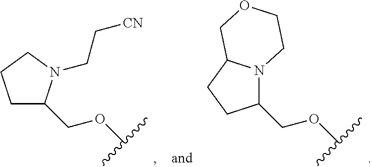 [SMILES: *OCC1CCCN1CCC#N ; *OCC1CCC2COCCN12]

C293. The compound of embodiment C292, wherein R¹ is selected from:
C294. The compound of any one of embodiments C256-C288, wherein R¹ is selected from

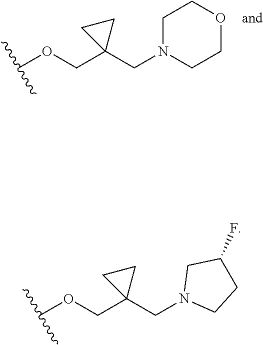 [SMILES: *OCC1(CN2CCOCC2)CC1 ; *OCC1(CN2CC[C@@H](F)C2)CC1]

C295. The compound of any one of embodiments C256-C294, wherein the compound is a compound according to Formula IA2, IB2, IC2, ID2, IE2, or IF2:

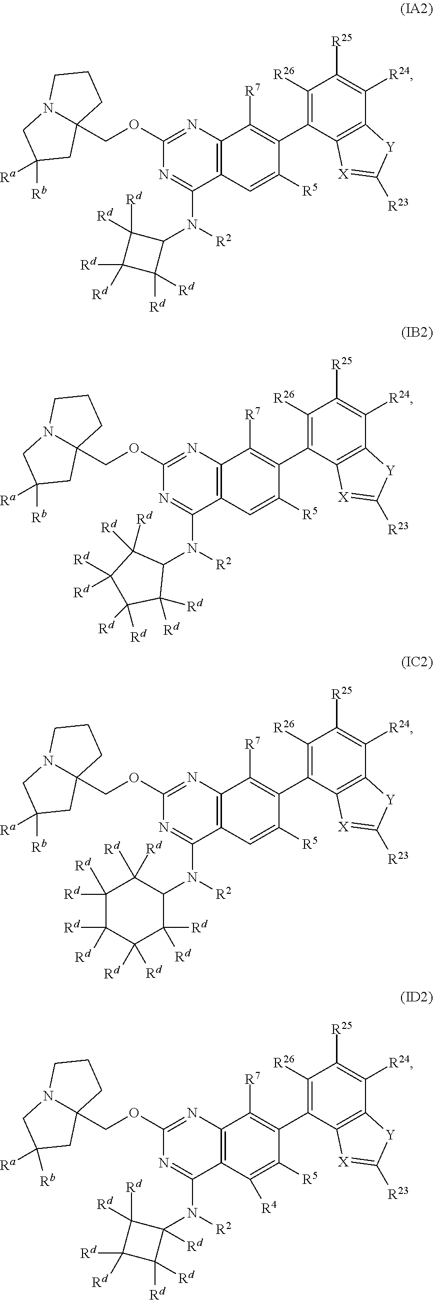

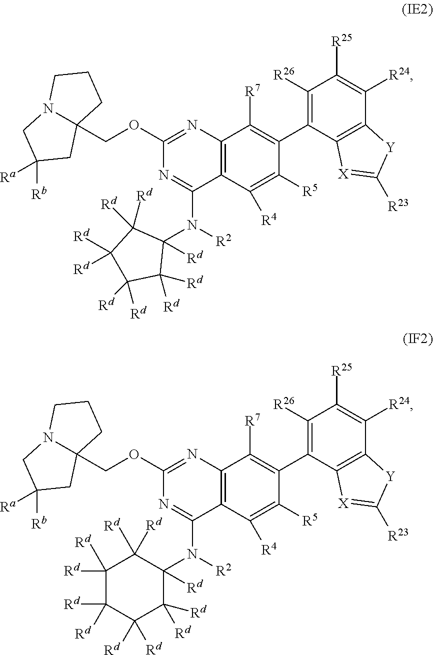

or a salt (e.g., pharmaceutically acceptable salt) thereof, wherein:
   R^{a} and R^{b} are each independently selected from halogen, C₁₋₆ alkyl, —OR¹², and H, wherein any C₁₋₆ alkyl is unsubstituted or is substituted with one or more
   each R^{d} is independently selected from H, —OR¹², —C(O)(C₁₋₆ alkylene)CN, —C(C₁₋₆ alkylene)OH, —C(O)(C₁₋₆alkyl), —C(O)N(R¹⁴)₂, —S(O)₂(C₁₋₆alkyl), halogen, and C₁₋₆ akyl, wherein any C₁₋₆ alkyl is unsubstituted or substituted with one or more R²⁰;
   X is selected from N and C—CN;
   Y is selected from O and S;
   R^{a} is selected from —N(R)₂, C₁₋₆alkyl, and C₁₋₆ alkyl-N(R¹⁴)₂, wherein any C₁₋₆ alkyl is unsubstituted or substituted with one or more R¹³; and
   R²⁴, R²⁵, and R²⁶ are independently selected from H, deuterium, halogen, and C₁₋₆ alkyl, wherein any C₁₋₆ alkyl is unsubstituted or substituted with one or more R²⁰.
C296. The compound of embodiment C295, wherein the compound is a compound according to Formula IA2, or a salt (e.g., pharmaceutically acceptable salt) thereof.
C297. The compound of embodiment C295, wherein the compound isa compound according to Formula B2, or a salt (e.g., pharmaceutically acceptable salt) thereof.
C298. The compound of embodiment C295, wherein the compound is a compound according to Formula IC2, or a salt (e.g., pharmaceutically acceptable salt) thereof.
C299. The compound of embodiment C295, wherein the compound is a compound according to Formula ID2, or a salt (e.g., pharmaceutically acceptable salt) thereof.
C300. The compound of embodiment C295, wherein the compound is a compound according to Formula IE2, or a salt (e.g., pharmaceutically acceptable salt) thereof.
C301. The compound of embodiment C295, wherein the compound is a compound according to Formula IF2, or a salt (e.g., pharmaceutically acceptable salt) thereof.
C302. The compound of any one of embodiments C295-C301, wherein R^{a} is a halogen (e.g., F).
C303. The compound of any one of embodiments C295-C302, wherein R^{b} is H.
C304. The compound of any one of embodiments C295-C303, wherein X is C—CN, Y is S, and R²³ is —N(R¹²)₂.
C305. The compound of any one of embodiments C295-C304, wherein one or more of R²⁴, R²⁵, and R²⁶ is a halogen (e.g., F).
C306. The compound of any one of embodiments C256-C305, wherein R² is H.
C307. The compound of any one of embodiments C256-C305, wherein R² is selected from C₁₋₆ alkyl that is unsubstituted or is substituted with one or more R¹³.
C308. The compound of embodiment C307, wherein R² is selected from C₂₋₆alkyl.
C309. The compound of any one of embodiments C256-C308, wherein R⁵ is H.
C310. The compound of any one of embodiments C256-C308, wherein R is a halogen (e.g., F or Cl).
C311. The compound of any one of embodiments C256-C308, wherein R⁵ is selected from C₁₋₆alkyl that is unsubstituted or substituted with one or more R¹³.
C312. The compound of embodiment C311, wherein R² is selected from C₁₋₆alkyl that is substituted with one or more halogens or —CN.
C313. The compound of embodiment C312, wherein R⁵ is selected from —CF₂H, —CF₃, —CH₂CN, and —CH₂CH₃.
C314. The compound of embodiment C313, wherein R⁵ is —CF₃.
C315. The compound of any one of embodiments C256-C314, wherein R⁷ is H.
C316. The compound of any one of embodiments C256-C314, wherein R⁷ is a halogen (e.g., F or Cl).
C317. The compound of any one of embodiments C256-C314, wherein R⁷ is —CN.
C318. A compound represented by Formula IV:

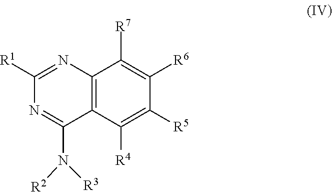

or a salt (e.g., pharmaceutically acceptable salt) thereof, wherein:
R¹ is selected from —OR⁸ and

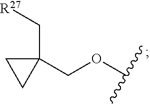

R² is selected from H, C₁₋₆alkyl, and a 3-6 membered carbocycle, wherein any C₁₋₆ alkyl is unsubstituted or is substituted with one or more R¹³;
R³ is selected from C₁₋₆alkyl that is unsubstituted or is substituted with one or mom R¹⁰;
R⁴ is H;
R⁵ is selected from H, halogen, —CN, —OR², a 3-6 membered heterocycle, a 5-6 membered heteroaryl, and C₁₋₆ alkyl, wherein any C₁₋₆alkyl is unsubstituted or substituted with one or more R¹³;
R⁶ is a bicyclic heteroaryl substituted with one or more R¹⁵;
R⁷ is selected from halogen, —CN, and H;
R⁸ is selected from a heterocycle and an alkylheterocycle, wherein any heterocycle comprises 4-8 members and is unsubstituted or is substituted with one or more R or R^{b}, and wherein an alkyl moiety of any alkylheterocycle is selected from C₁₋₆alkyl;
each R¹⁰ is independently selected from —OR¹⁴, ═O, —CN, —N(R¹⁴)₂, a 3-6 membered carbocycle, a 5-6 membered heteroaryl, —S(O)₂(C₁₋₆ alkyl), —N(R¹)C(O)(C₁₋₆alkyl), C₁₋₆alkyl, and halogen, wherein any C₁₋₆alkyl is unsubstituted or substituted with one or more R, any 3-6 membered carbocycle is unsubstituted or substituted with one or more R², and any 5-6 membered heteroaryl is unsubstituted or substituted with one or more R¹³;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹², —CN, —N(R¹⁴)₂, and halogen;
each R¹⁴ is independently selected from C₁₋₆ alkyl, C₂₋₆ alkenyl, and H;
each R¹⁵ is independently selected from deuterium, halogen, —N(R¹⁴)₂, —CN, and C₁₋₆alkyl, wherein any C₁₋₆alkyl is unsubstituted or substituted with one or more R¹³;
each R²⁰ is independently selected from —OH, —OC₁₋₆ alkyl, —CN, —NH₂. —NHC₁₋₆alkyl, and halogen;
R²⁷ is a 3-6 membered heterocycle including one or more heteroatoms selected from N, O, and S, wherein the heterocycle is unsubstituted or substituted with one or more R²¹;
each R²⁸ is independently selected from C₁₋₆alkyl and halogen; and
R^{a} and R^{b} are each independently selected from halogen, C₁₋₆ alkyl, —OR¹², and H, wherein any C₁₋₆alkyl is unsubstituted or is substituted with one or more R¹³, and wherein an R^{a} and R^{b} optionally join together to form a 3-6 membered carbocycle or heterocycle
C319. The compound of embodiment C318, wherein the compound is a compound according to Formula IVA:

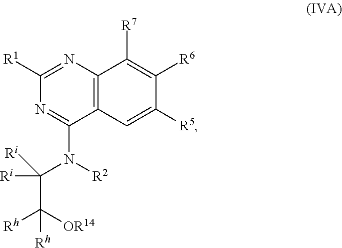

or a salt (e.g., pharmaceutically acceptable salt) thereof, wherein:
each Rⁱ and R^{j} are independently selected from H and R¹⁰.
C320. The compound of embodiment C319, wherein each Rⁱ and R^{h} are independently selected from H and C₁₋₆alkyl, wherein any C₁₋₆alkyl is unsubstituted or substituted with one or more R²⁰.
C321. The compound of embodiment C319 or C320, wherein R¹⁴ is H.
C322. The compound of any one of embodiments C318-C321, wherein R¹ is selected from:

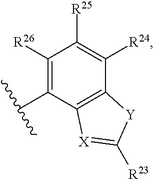

wherein:
   X is selected from N and C—CN;
   Y is selected from O and S;
   R² is selected from —N(R¹²)₂, C₁₋₆alkyl, and C₁₋₆alkyl-N(R¹⁴)₂, wherein any C₁₋₆akyl is unsubstituted or substituted with one or more R¹³; and
   R²⁴, R²⁵, and R²⁶ are independently selected from H, deuterium, halogen, and C₁₋₆alkyl, wherein any C₁₋₆alkyl is unsubstituted or substituted with one or more R¹³.
C323. The compound of any one of embodiments C318-C322, wherein R⁵ is selected from:

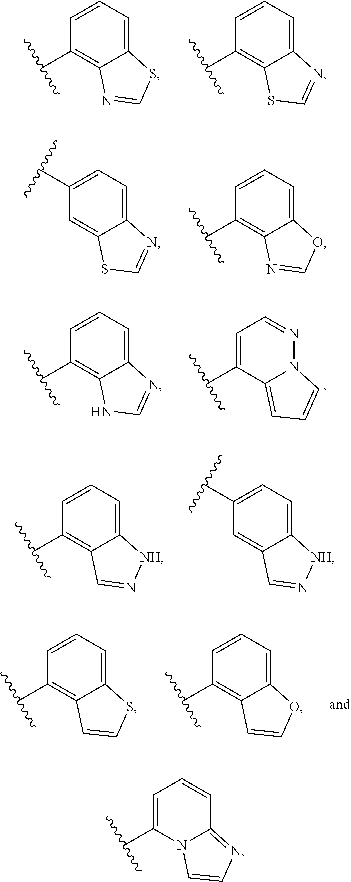

any of which is substituted with one or more R¹⁵.

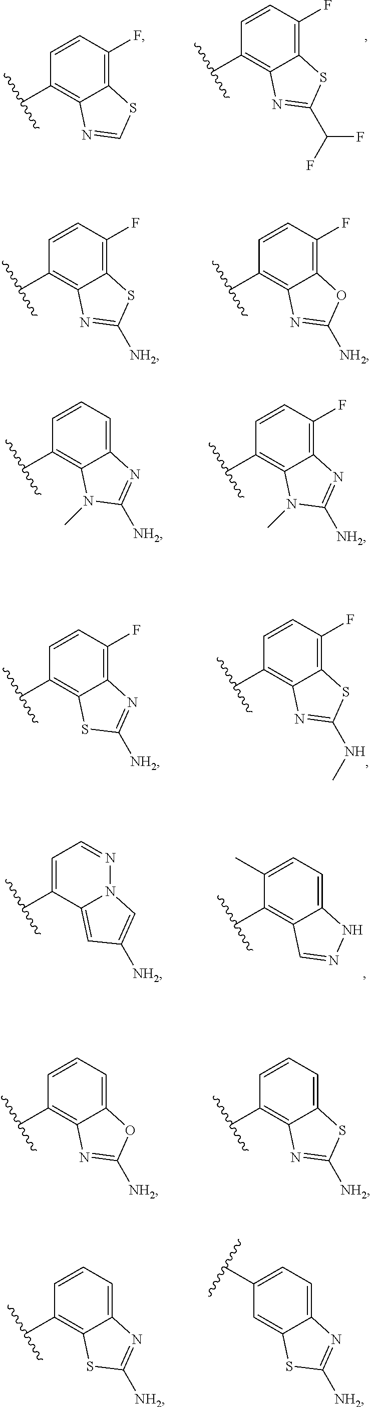

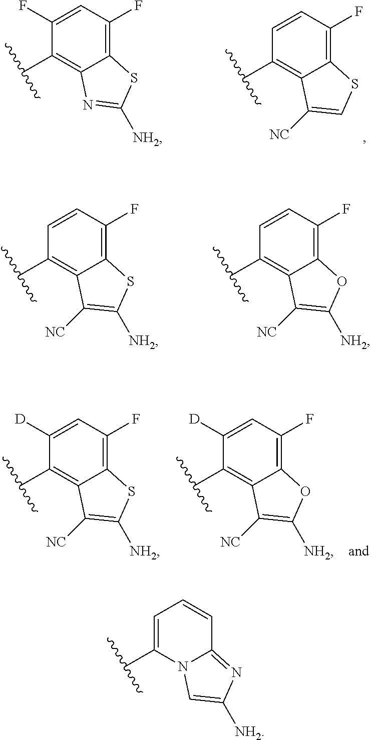

C324. The compound ofany one of embodiments C318-C323, wherein R⁶ is selected from:

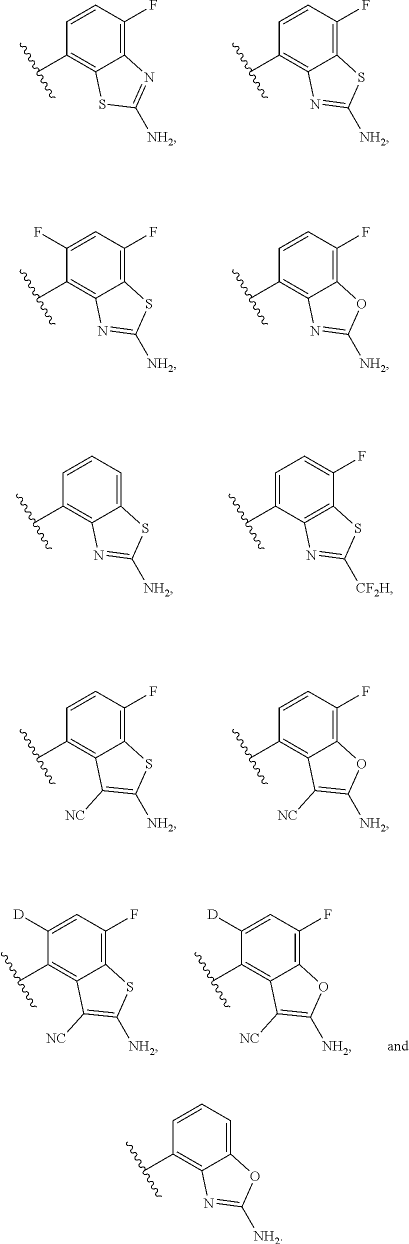

C326. The compound of embodiment C318, wherein the compound is a compound according to Formula IVB:

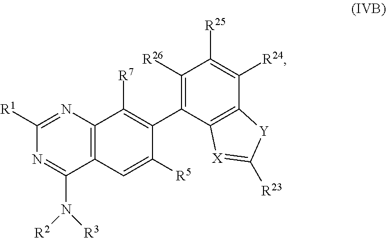

or a salt (e.g., pharmaceutically acceptable salt) thereof, wherein:
X is selected from N and C—CN;
Y is selected from O and S;
R² is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, deuterium, halogen, —OR¹², and C₁₋₆alkyl, wherein any C₁₋₆alkyl is unsubstituted or substituted with one or more R¹³.
C327. The compound of any one of embodiments C318-C326, wherein R¹ is selected from —OR⁸.
C328. The compound of embodiment C327, wherein R¹ is selected from:

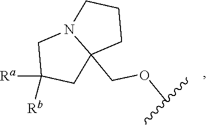

wherein R^{a} and R^{b} are each independently selected from halogen, C₁₋₆alkyl, —OR¹², and H, wherein any C₁₋₆alkyl is unsubstituted or is substituted with one or more R¹³.
C329. The compound of embodiment C322, wherein R¹ is selected from:

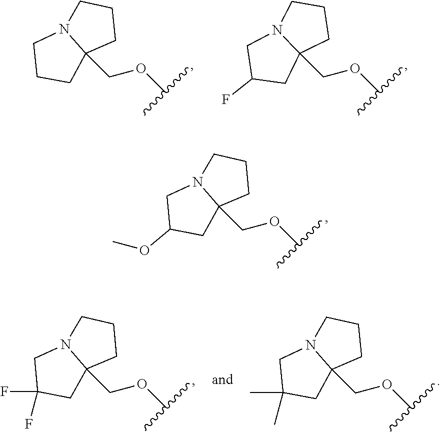 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

C330. The compound of embodiment C327, wherein R¹ is selected from:

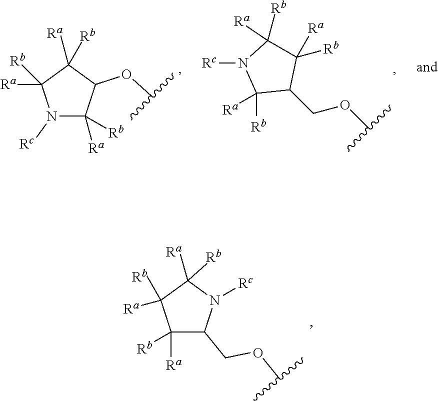

wherein each R^{a} and R^{b} is independently selected from halogen, C₁₋₆ alkyl, —OR¹², and H; and R^{c} is selected from C₁₋₆alkyl, wherein any C₁₋₆ alkyl is unsubstituted or is substituted with one or more R¹³, and wherein an R^{a} and R^{b} or R^{c} optionally join together to form a 3-6 membered carbocycle or heterocycle.
C331. The compound of embodiment C330, wherein R¹ is selected from:

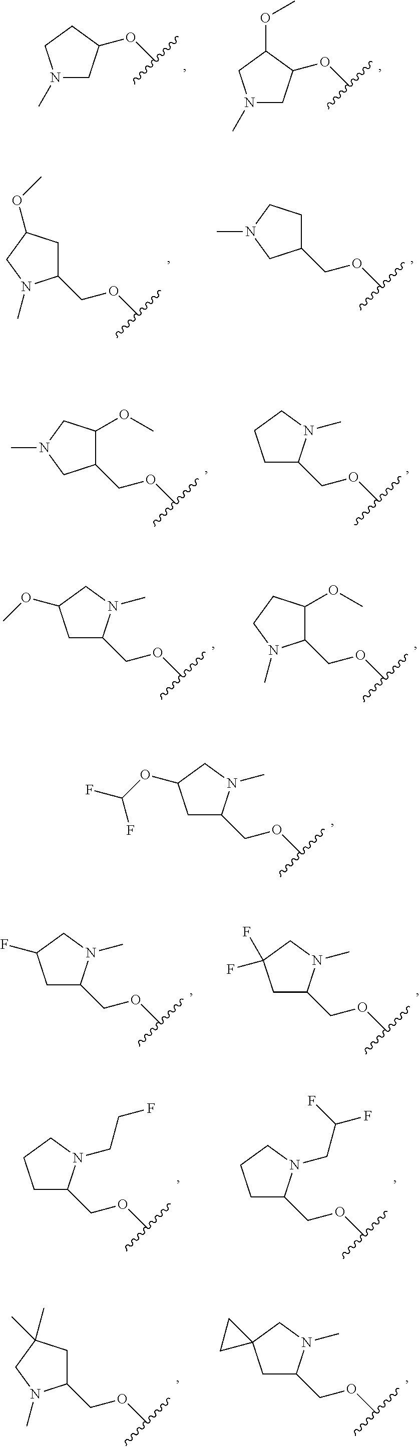

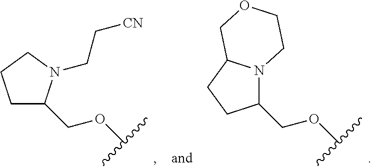 [SMILES: *OCC1CCCN1CCC#N ; *OCC1CCC2COCCN12]

C332. The compound of any one of embodiments C318-C326, wherein R¹ is selected from

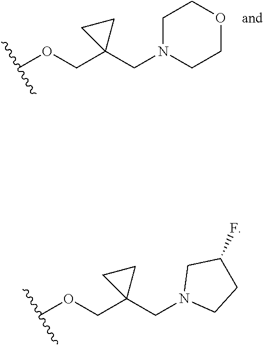 [SMILES: *OCC1(CN2CCOCC2)CC1 ; *OCC1(CN2CC[C@@H](F)C2)CC1]

C333. The compound of any one of embodiments C318-C332, wherein the compound is a compound according to Formula IVC:

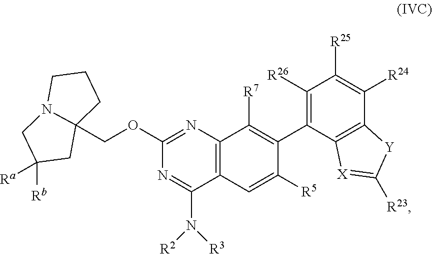

or a salt (e.g., pharmaceutically acceptable salt) thereof, wherein:
R^{a} and R^{b} are each independently selected from halogen, C₁₋₆ alkyl, —OR², and H, wherein any C₁₋₆alkyl is unsubstituted or is substituted with one or more R¹³;
X is selected from N and C—CN;
Y is selected from O and S;
R² is selected from —N(R¹²)₂, C₁₋₆alkyl, and C₁₋₆alkyl-N(R¹)₂, wherein any C₁₋₆alkyl is unsubstituted or substituted with one or more R¹³; and
R²⁴, R²⁵, and R²⁶ are independently selected from H, deuterium, halogen, —OR¹², and C₁₋₆alkyl, wherein any C₁₋₆alkyl is unsubstituted or substituted with one or more R¹³.
C334. The compound of embodiment C333, wherein R¹ is a halogen (e.g., F).
C335. The compound of embodiment C333 or C334, wherein R is H.
C336. The compound of any one of embodiments C326-C335, wherein X is C—CN, Y is S, and R² is selected from —N(R¹²)₂.
C337. The compound of any one of embodiments C326-C336, wherein at least one of R²⁴, R²⁵, and R²⁶ is a halogen (e.g., F).
C338. The compound of any one of embodiments C318 and C326-C337, wherein R³ is selected from C₁₋₃ alkyl that is unsubstituted or is substituted with one or more R²⁰.
C339. The compound of embodiment C338, wherein each R¹⁰ is independently selected from —OR¹², ═O, —CN, —NH(R¹⁶), —N(R¹⁶)₂, a 3-6 membered carbocycle, C₁₋₆alkyl, and halogen, wherein any C₁₋₆ alkyl is unsubstituted or substituted with one or more R²⁰, wherein any 3-6 membered carbocycle is unsubstituted or substituted with one or more R¹², and wherein each R¹⁶ is independently selected from C₁₋₆alkyl and C₂₋₆alkenyl.
C340. The compound of embodiment C339, wherein each R¹⁰ is independently selected from —OR¹², —CN, C₁₋₆alkyl, and halogen, wherein any C₁₋₆alkyl is unsubstituted or substituted with one or more R².
C341. The compound of embodiment C340, wherein each R¹⁰ is independently selected from halogen.
C342. The compound of embodiment C338, wherein R³ is selected from C₁₋₃alkyl that is unsubstituted.
C343. The compound of any one of embodiments C318 and C326-C337, wherein the moiety

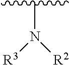

is selected from:

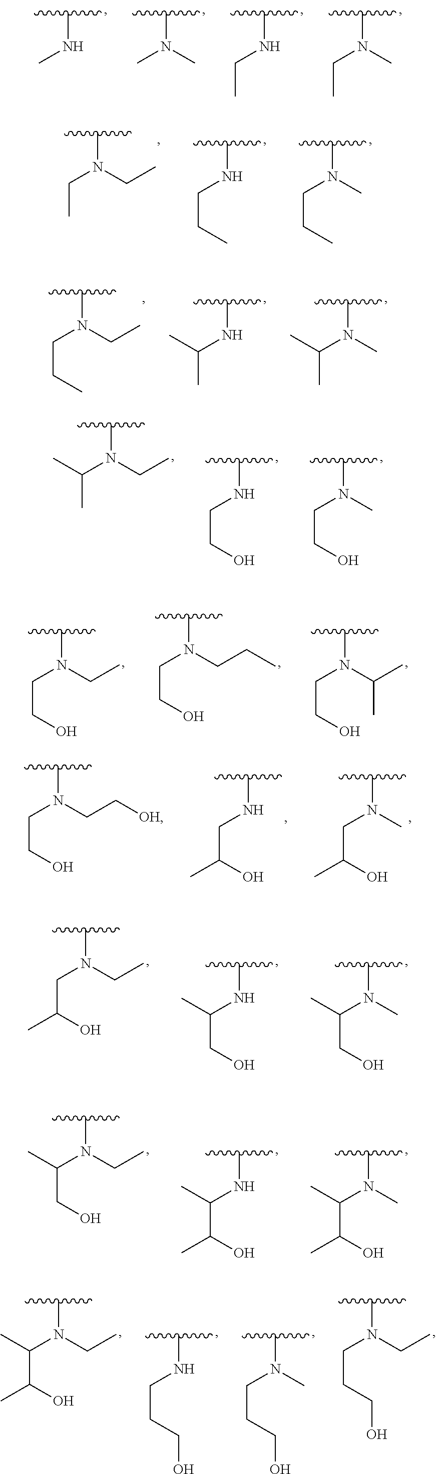

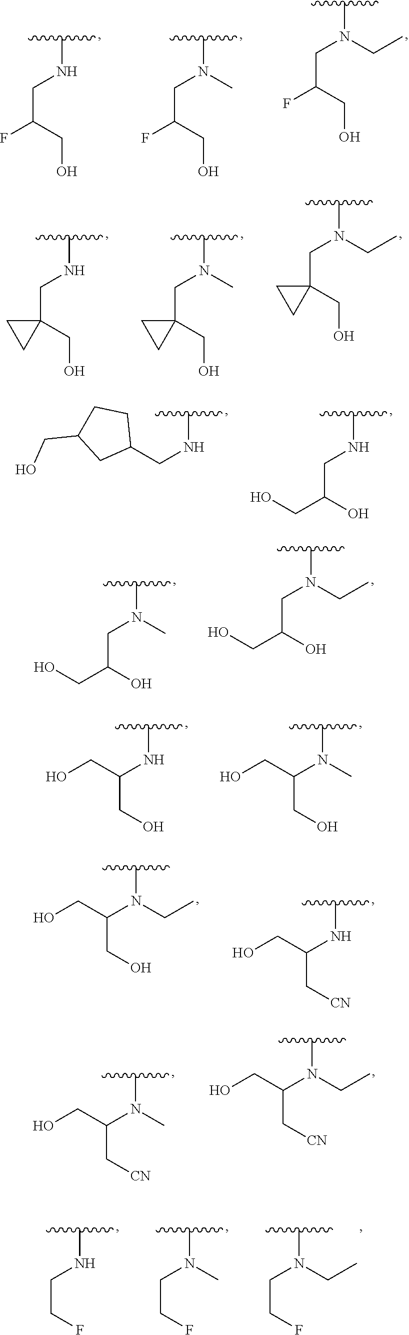

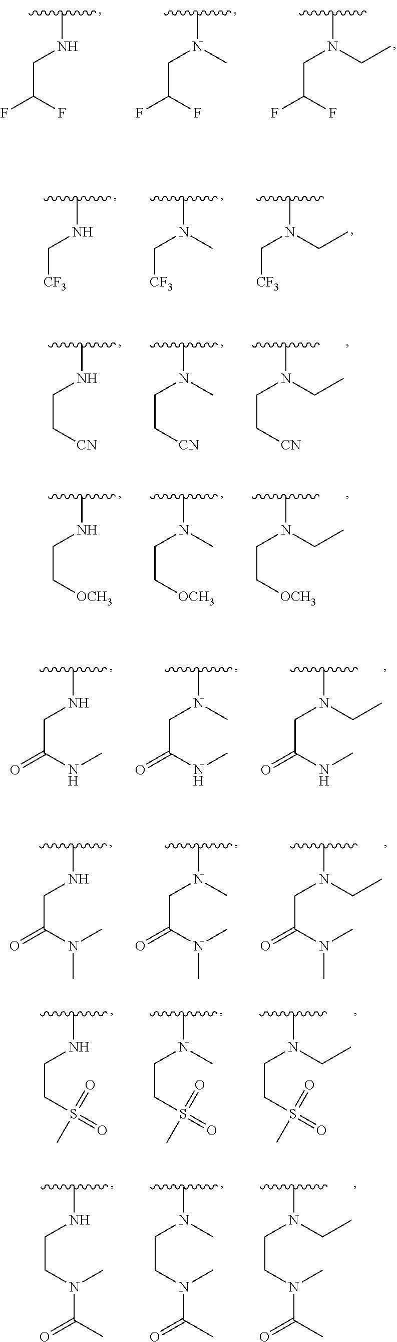

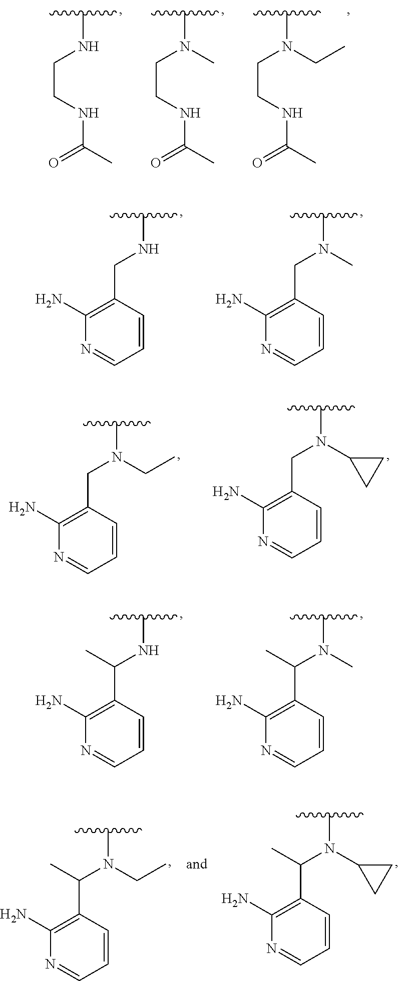

any of which is optionally further substituted with one or more R²⁰.
C344. The compound of any one of embodiments C318-C343, wherein R² is H.
C345. The compound of any one of embodiments C318-C343, wherein R² is selected from C₁₋₆alkyl that is unsubstituted or is substituted with one or more R¹¹
C346. The compound of embodiment C345, wherein R² is selected from C₁₋₂alkyl that is unsubstituted.
C347. The compound of any one of embodiments C318-C343, wherein R² is a 3-6 membered carbocycle.
C348. The compound of any one of embodiments C318-C346, wherein R⁵ is H.
C349. The compound of any one of embodiments C318-C346, wherein R⁵ is a halogen (e.g., F or Cl).
C350. The compound of any one of embodiments C318-C346, wherein R⁵ is selected from C₁₋₆ alkyl that is unsubstituted or substituted with one or more R¹³.
C351. The compound of embodiment C350, wherein R¹ is selected from C₁₋₆alkyl that is substituted with one or more halogens or —CN.
C352. The compound of embodiment C351, wherein R¹ is selected from —CF₂H, —CF, —CH₂CN, and —CH₂CH₁.
C353. The compound of embodiment C352, wherein R⁵ is —CF₃.
C354. The compound of any one of embodiments C318-C353, wherein R⁷ is H.
C355. The compound of any one of embodiments C318-C353, wherein R⁷ is a halogen (e.g., F or Cl).
C356. The compound of any one of embodiments C318-C353, wherein R⁷ is —CN.
C357. The compound of any one of embodiments C318-C356, wherein the compound is a not a compound included in Table 3.
C358. A compound represented by Formula V:

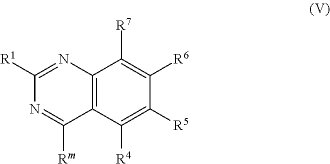

or a salt (e.g., pharmaceutically acceptable salt) thereof, wherein:
R¹ is selected from —OR⁸ and

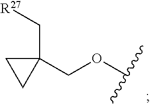

Rⁿ is selected from hydrogen and C₁₋₆alkyl, wherein any C₁₋₆alkyl is unsubstituted or substituted with one or more R¹³;
R⁴ is H;
R⁵ is selected from halogen and C₁₋₆alkyl, wherein any C₁₋₆alkyl is unsubstituted or substituted with one or more R¹³;
R⁶ is a monocyclic or bicyclic heteroaryl, wherein the heteroaryl is substituted with one or more R¹⁵;
R⁷ is selected from halogen;
R⁸ is selected from a heterocycle and an alkylheterocycle, wherein any heterocycle comprises 4-8 members and is unsubstituted or is substituted with one or more R^{a} or R^{b}, and wherein an alkyl moiety of any alkylheterocycle is selected from C₁₋₆alkyl;
each R² is independently selected from C₁₋₆ alkyl, C₂, alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₄ alkyl, C₂₋₄ alkenyl, and H;
each R¹⁵ is independently selected from deuterium, halogen, —N(R¹²)₂, —CN, and C₁₋₆alkyl, wherein any C₁₋₆alkyl is unsubstituted or substituted with one or more R¹³;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen; and
R^{a} and R^{b} are each independently selected from deuterium, halogen, C₁₋₆ alkyl, a 3-6 membered carbocycle, —OR², and H, wherein an R and R^{b} optionally join together to form a 3-6 membered carbocycle or heterocycle, and wherein any C₁₋₆alkyl or 3-6 membered carbocycle or heterocycle is unsubstituted or is substituted with one or more R¹³.
C359. The compound of embodiment C358, wherein R^{f} is hydrogen.
C360. The compound of embodiment C358 or C359, wherein the compound is a compound according to Formula VA:

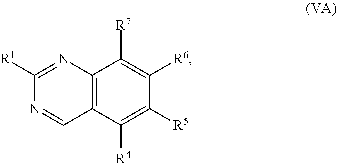

or a salt (e.g., pharmaceutically acceptable salt) thereof.
C361. The compound of embodiment C358, wherein the compound is a compound according to Formula VB:

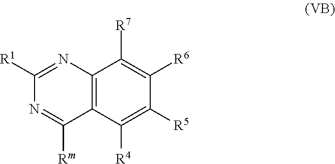

or a salt (e.g., pharmaceutically acceptable salt) thereof, wherein:
Rⁿ is C₁₋₆alkyl that is unsubstituted or substituted with one or more R¹³.
C362. The compound of embodiment C358 or C361, wherein R¹¹ is C₁₋₆alkyl that is unsubstituted or substituted with one or more R¹³.
C363. The compound of embodiment C362, wherein R^{f} is C₁₋₆alkyl that is unsubstituted.
C364. The compound of any one of embodiments C358-C363, wherein R⁵ is a bicyclic heteroaryl that is substituted with one or more R¹³.
C365. The compound of embodiment C364, wherein R¹ is selected from:

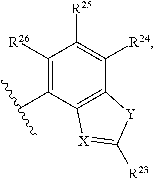

wherein X is selected from N and C—CN; Y is selected from O and S; R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and R²⁴, R¹¹, and R² are independently selected from H, deuterium, halogen, and C₁₋₆ alkyl, wherein any C₁₋₆alkyl is unsubstituted or substituted with one or more R¹³.
C366. The compound of embodiment C365, wherein X is C—CN; Y is S; and R² is —N(R¹²)₂.
C367. Ile compound of embodiment C365 or C366, wherein at least one of R²⁴, R²⁵, and R²⁶ is halogen.
C368. The compound of any one of embodiments C358-C363, wherein R¹ is a monocyclic heteroaryl that is substituted with one or more R¹³.
C369. The compound of embodiment C368, wherein R¹ is a pyridine substituted with one or more R¹³.
C370. The compound of embodiment C369, wherein R has the structure

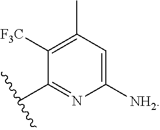 [SMILES: *c1nc(N)cc(C)c1C(F)(F)F]

C371. The compound of any one of embodiments C358-C370, wherein the compound is a compound according to Formula VC:

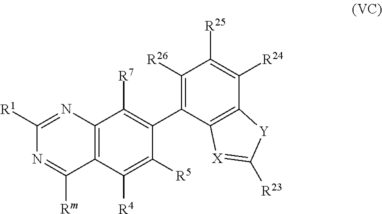

or a salt (e.g., pharmaceutically acceptable salt) thereof, wherein:
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, deuterium, halogen, —OR¹², and C₁₋₆alkyl, wherein any C₁₋₆alkyl is unsubstituted or substituted with one or more R¹³.
C372. The compound of embodiment C371, wherein X is C—CN, Y is S, and R² is selected from —N(R¹²)₂.
C373. The compound of embodiment C371 or C372, wherein at least one of R²⁴, R¹¹, and R² is halogen.
C374. The compound of any one of embodiments C358-C373, wherein R¹ is selected from —OR⁸.
C375. The compound of embodiment C374, wherein R¹ is selected from:

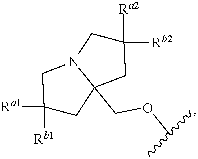

wherein R^{a1}, R^{a2}, R^{b1}, and R^{b2} are each independently selected from deuterium, halogen, C₁₋₆alkyl, —OR², and H, wherein R^{a2} and R^{b2} can optionally join together to form a 3-6 membered carbocycle, and wherein any C₁₋₆alkyl or 3-6 membered carbocycle is unsubstituted or is substituted with one or more R¹³.
C376. The compound of embodiment C375, wherein R¹ is selected from:

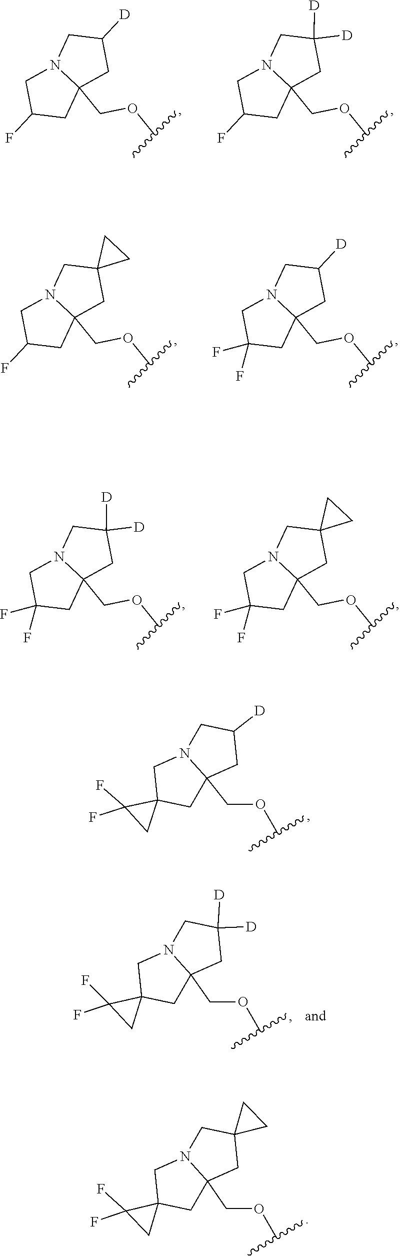

C377. The compound of embodiment C374, wherein R¹ is selected from:

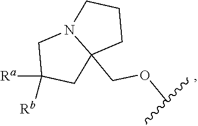

wherein R^{a} and R^{b} are each independently selected from halogen, C₁₋₆ alkyl, —OR¹², and H, wherein any C₁₋₆alkyl is unsubstituted or is substituted with one or more R¹³.
C378. The compound of embodiment C377, wherein R¹ is selected from:

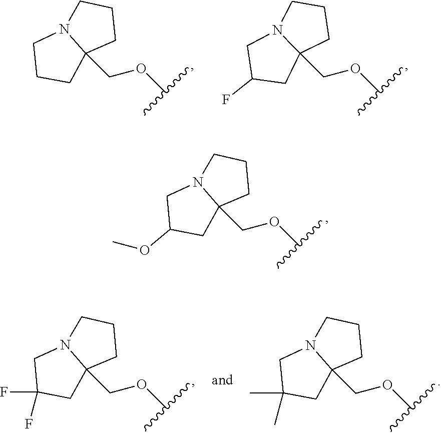 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

C379. The compound of embodiment C374, wherein R¹ is selected from:

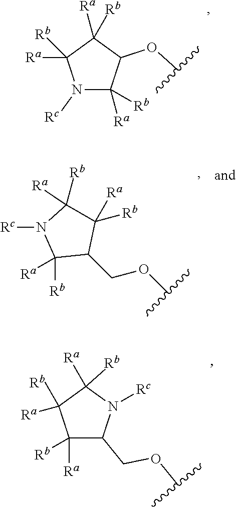

wherein each R^{a} and R^{b} is independently selected from halogen, C₁₋₆ alkyl, —OR¹², and H; and R^{c} is selected from C₁₋₆ alkyl, wherein an R^{a} and R^{b} or R^{c} optionally join together to form a 3-6 membered carbocycle or heterocycle, and wherein any C₁₋₆ alkyl or 3-6 membered carbocycle or heterocycle is unsubstituted or is substituted with one or more R¹³.
C380 The compound of embodiment C379, wherein R¹ is selected from:

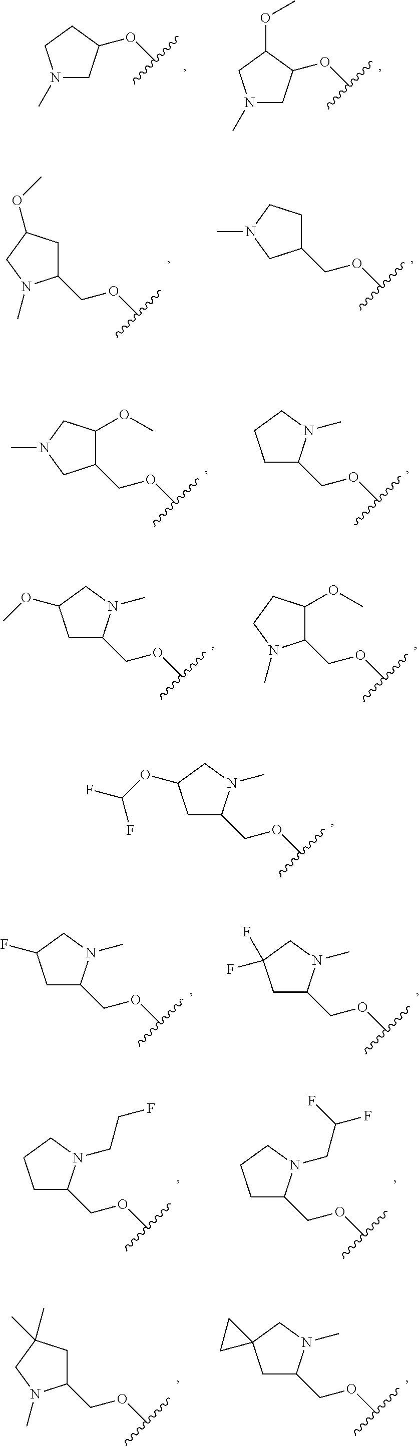

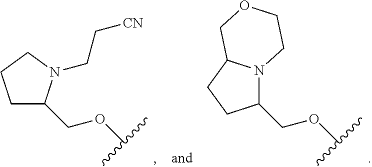 [SMILES: *OCC1CCCN1CCC#N ; *OCC1CCC2COCCN12]

C381. The compound of any one of embodiments C358-C373, wherein R¹ is selected from

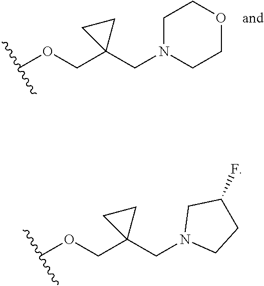 [SMILES: *OCC1(CN2CCOCC2)CC1 ; *OCC1(CN2CC[C@@H](F)C2)CC1]

C382. The compound of any one of embodiments C358-C381, wherein R⁵ is a halogen (e.g., F or Cl).
C383. The compound of any one of embodiments C358-C381, wherein R³ is selected from C₁₋₆alkyl that is unsubstituted or substituted with one or more R¹³.
C384. The compound of embodiment C383, wherein R⁵ is selected from C₁₋₆alkyl that is substituted with one or more halogens or —CN.
C385. The compound of embodiment C384, wherein R⁵ is selected from —CF₂H, —CF₃, —CH₂CN, and —CH₂CH₃.
C386. The compound of embodiment C385, wherein R⁵ is —CF₃.
C387. A compound represented by Formula VI:

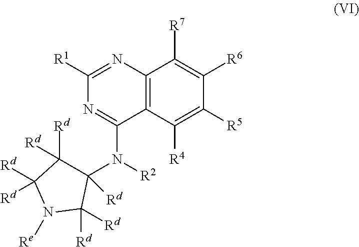

or a salt (e.g., pharmaceutically acceptable salt) thereof, wherein:
R¹ is selected from —OR and

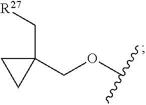

R² is selected from H, C₁₋₆alkyl, and a 3-6 membered carbocycle, wherein any C₁₋₆ alkyl is unsubstituted or is substituted with one or more R¹³;
R⁴ is H;
R⁵ is selected from H, halogen, —CN, —OR², a 3-6 membered heterocycle, a 5-6 membered heteroaryl, and C₁₋₆alkyl, wherein any Cr-alkyl is unsubstituted or substituted with one or more R¹³;
R⁶ is a monocyclic or bicyclic heteroaryl, wherein the heteroaryl is substituted with one or more R¹³;
R⁷ is selected from H and halogen;
R⁸ is selected from a heterocycle and an alkylheterocycle, wherein any heterocycle comprises 4-8 members and is unsubstituted or is substituted with one or more R or R, and wherein an alkyl moiety of any alkylheterocycle is selected from C₁₋₆akyl;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹², —CN, —N(R¹⁴)₂, and halogen;
each R¹⁴ is independently selected from a 3-6 membered carbocycle, a 3-6 membered heterocycle, C₁₋₆ alkyl, C₂₋₆ alkenyl, and H, wherein any C₁₋₆alkyl is optionally deuterated;
each R¹⁵ is independently selected from deuterium, halogen, —N(R¹⁴)₂, —CN, and C₁₋₆alkyl, wherein any C₁₋₆alkyl is unsubstituted or substituted with one or more R¹³;
each R² is independently selected from —OH, —OC₁₋₆alkyl, ═O, —CN, —NH₂, —NHC₁₋₆alkyl, —C(O)C₁₋₆alkyl), a 3-6 membered carbocycle, a 5-6 membered aryl, and halogen;
R²⁷ is a 3-6 membered heterocycle including one or more heteroatoms selected from N, O, and S, wherein the heterocycle is unsubstituted or substituted with one or more R²⁸;
each R²⁸ is independently selected from C₁₋₆alkyl and halogen;
R^{a} and R^{b} are each independently selected from deuterium, halogen, C₁₋₆ alkyl, —OR², a 3-6 membered carbocycle, and H, wherein an R^{a} and R^{b} optionally join together to form a 3-6 membered carbocycle or heterocycle, and wherein any C₁₋₆alkyl or 3-6 membered carbocycle or heterocycle is unsubstituted or is substituted with one or more R¹³;
each R^{d} is independently selected from H, halogen, and C₁₋₆alkyl, wherein any C₁₋₆alkyl is unsubstituted or substituted with one or more R²⁰;
R^{c} is selected from

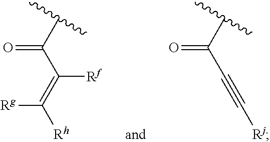

R^{f} is selected from H, halogen, and C₁₋₆alkyl;
R^{g} and R^{h} are independently selected from H and C₁₋₆alkyl; and
R^{j} is selected from C₁₋₆alkyl,
provided that R^{e} is not

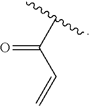 [SMILES: *C(=O)C=C]

C388. The compound of embodiment C387, wherein R^{e} is

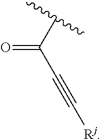

C389. The compound of embodiment C388, wherein Rⁱ is C₁₋₃ alkyl.
C390. The compound of embodiment C387, wherein R^{e} is selected from

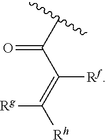

C391. The compound of embodiment C390, wherein R^{g} and R^{h} are H, and R^{f} is not H.
C392. The compound of embodiment C390, wherein R^{g} is H, and at least one of R^{h} and R^{f} is not H.
C393. The compound of embodiment C390, wherein R^{f} is H, and at least one of R^{g} and R^{h} is not H.
C394. The compound of embodiment C390, wherein R^{e} is

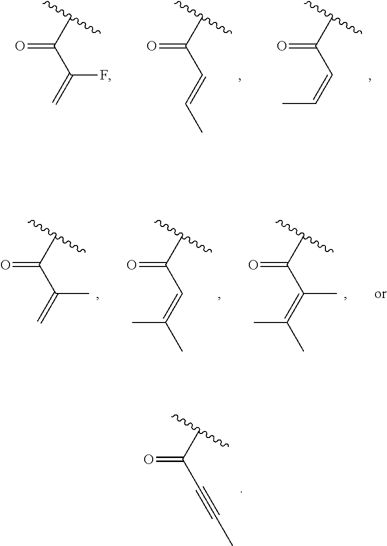

C395. The compound of any one of embodiments C387-C394, wherein each R^{d} is H.
C396. The compound of any one of embodiments C387-C394, wherein one R^{d} is selected from C₁₋₆alkyl and each other R^{d} is H.
C397. The compound of any one of embodiments C387-C396, wherein R⁶ is a bicyclic heteroaryl that is substituted with one or more R¹³.
C398. The compound of embodiment C397, wherein R⁶ is selected from:

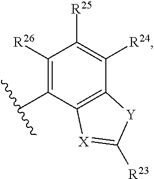

wherein X is selected from N and C—CN; Y is selected from O and S; R²³ is selected from —N(R¹²)₂, C₁₋₆alkyl, and C₁₋₆alkyl-N(R¹⁴)₂, wherein any C₁₋₄akyl is unsubstituted or substituted with one or more R¹³; and R²⁴, R⁵, and R⁶ are independently selected from H, deuterium, halogen, and C₁₋₆alkyl, wherein any C₁₋₆alkyl is unsubstituted or substituted with one or more R¹³.
C399. The compound of embodiment C398, wherein X is C—CN; Y is S; and R²³ is —N(R¹²)₂.
C400. The compound of embodiment C398 or C399, wherein at least one of R²⁴, R²⁵, and R²⁶ is halogen.
C401. The compound of any one of embodiments C387-C400, wherein R⁶ is selected from:

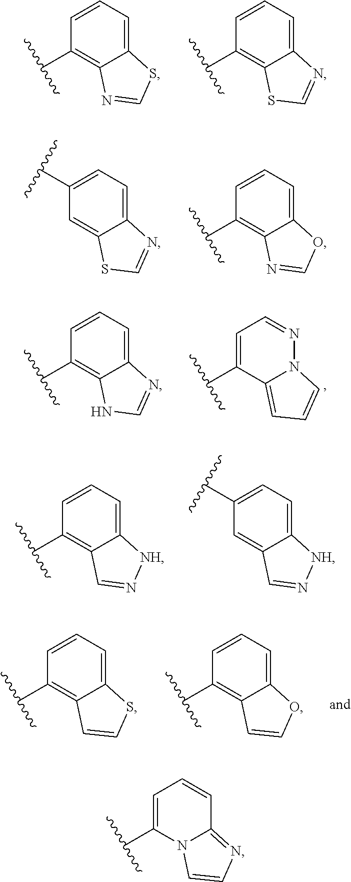

any of which is substituted with one or more R¹³.
C402. The compound ofany one of embodiments C387-C401, wherein R¹ is selected from:

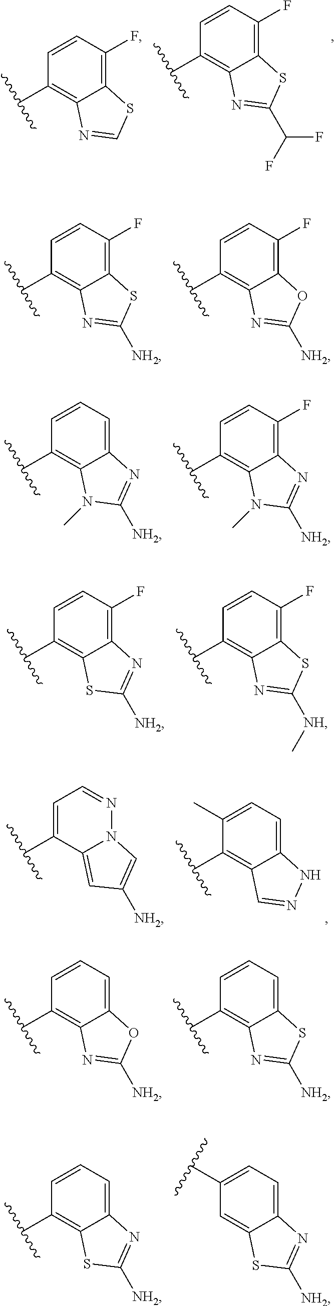

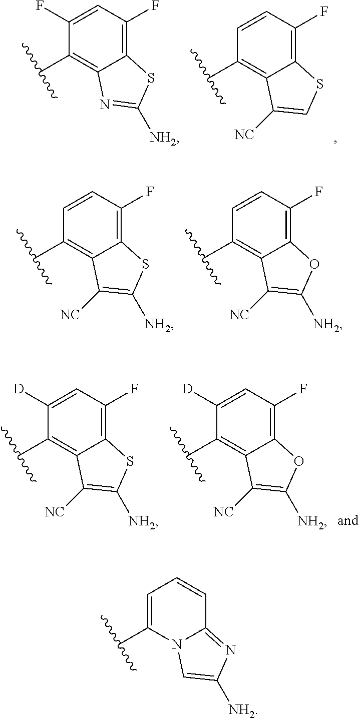

C403. The compound of any one of embodiments C387-C402, wherein R¹ is selected from:

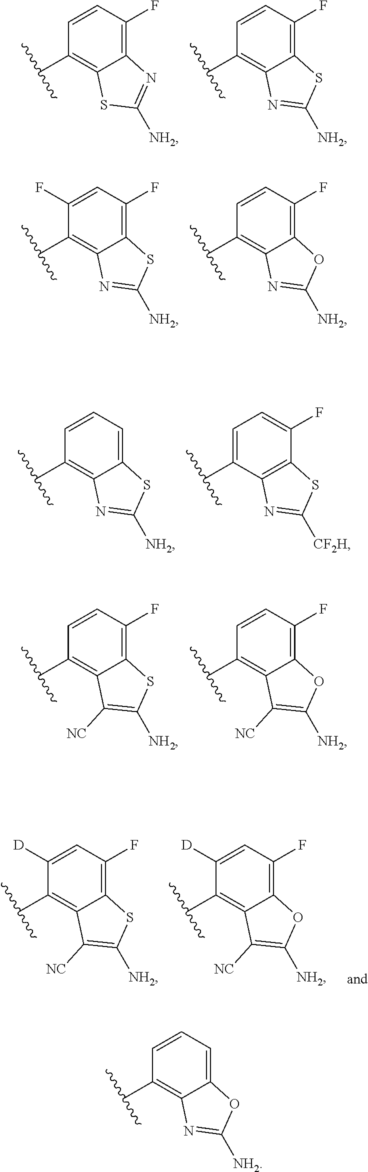

C404. The compound of any one of embodiments C387-C396, wherein R¹ is a monocyclic heteroaryl that is substituted with one or more R⁵.
C405. The compound of embodiment C404, wherein R⁶ is a pyridine substituted with one or more R¹⁵.
C406. The compound of embodiment C405, wherein R has the structure

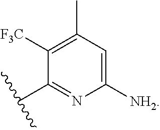 [SMILES: *c1nc(N)cc(C)c1C(F)(F)F]

C407. The compound of any one of embodiments C387-C406, wherein R¹ is selected from —OR⁸.
C408. The compound of embodiment C407, wherein R¹ is selected from:

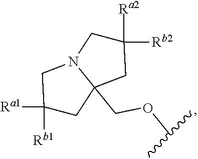

wherein R^{a1}, R^{a2}, R^{b1}, and R^{b2} are each independently selected from deuterium, halogen, C₁₋₆alkyl, —OR¹², and H, wherein R^{a2} and R^{b2} can optionally join together to form a 3-6 membered carbocycle, and wherein any C₁₋₆alkyl or 3-6 membered carbocycle is unsubstituted or is substituted with one or more R¹³.
C409. The compound of embodiment C408, wherein R¹ is selected from:

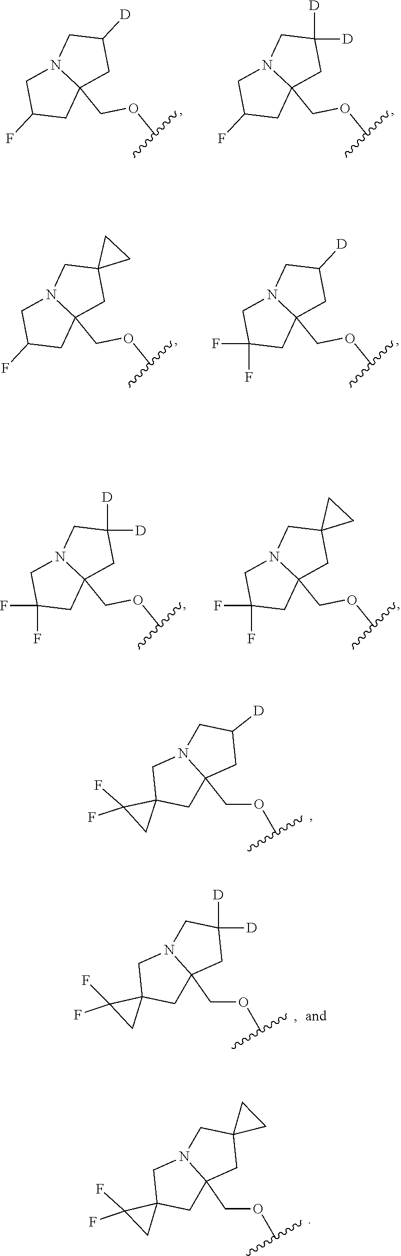

C410. The compound of embodiment C407, wherein R¹ is selected from:

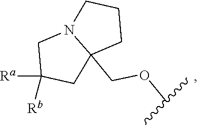

wherein R^{a} and R^{b} arm each independently selected from halogen, C₁₋₆alkyl, —OR¹², and H, wherein any C₁₋₆ alkyl is unsubstituted or is substituted with one or more R¹³.
C411. The compound of embodiment C410, wherein R¹ is selected from:

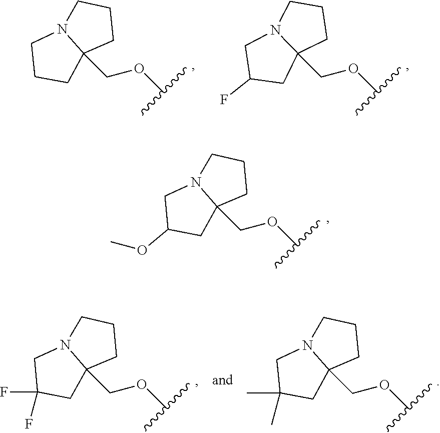 [SMILES: *OCC12CCCN1CCC2 ; *OCC12CCCN1CC(F)C2 ; *OCC12CCCN1CC(OC)C2 ; *OCC12CCCN1CC(F)(F)C2 ; *OCC12CCCN1CC(C)(C)C2]

C412. The compound of embodiment C407, wherein R¹ is selected from:

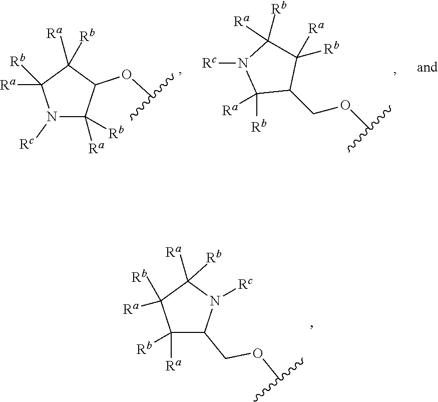

wherein each R^{a} and R^{b} is independently selected from halogen, C₁₋₆ alkyl, —OR¹², and H; and R is selected from C₁₋₆alkyl, wherein an R^{a} and R^{b} or R^{c} optionally join together to form a 3-6 membered carbocycle or heterocycle, and wherein any C₁₋₆ alkyl or 3-6 membered carbocycle or heterocycle is unsubstituted or is substituted with one or more R¹³.
C413. The compound of embodiment C412, wherein R¹ is selected from:

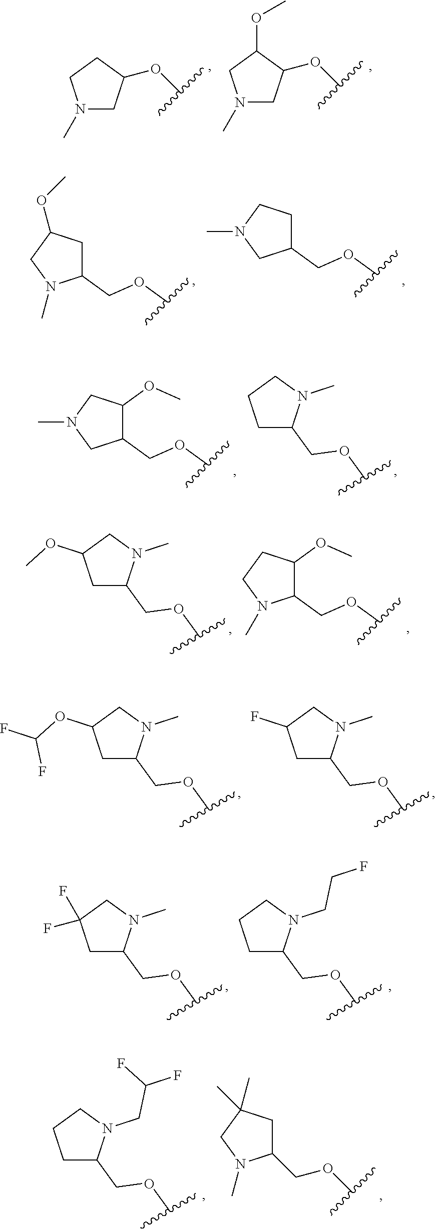

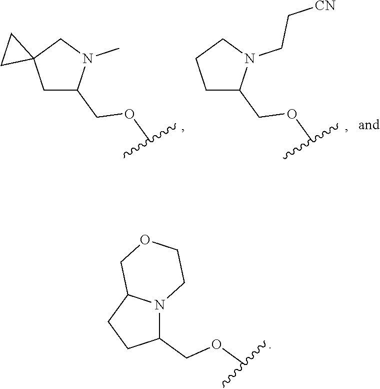 [SMILES: *OCC1CC2(CC2)CN1C ; *OCC1CCCN1CCC#N ; *OCC1CCC2COCCN12]

C414. The compound of any one of embodiments C387-C406, wherein R¹ is selected from

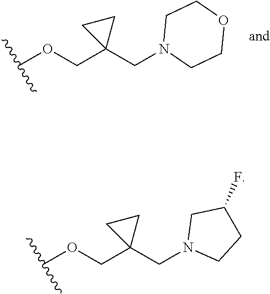 [SMILES: *OCC1(CN2CCOCC2)CC1 ; *OCC1(CN2CC[C@@H](F)C2)CC1]

C415. The compound of any one of embodiments C387-C414, wherein R² is H.
C416. The compound of any one of embodiments C387-C414, wherein R² is C₁₋₆ alkyl, wherein any C₁₋₄ alkyl is unsubstituted or is substituted with one or more R¹³.
C417. The compound of any one of embodiments C387-C414, wherein R² is a 3-6 membered carbocycle.
C418. The compound of any one of embodiments C387-C417, wherein R⁵ is a halogen (e.g., F or Cl).
C419. The compound of any one of embodiments C387-C417, wherein R⁵ is selected from C₁₋₆alkyl that is unsubstituted or substituted with one or more R¹³.
C420. The compound of embodiment C419, wherein R⁵ is selected from C₁₋₆alkyl that is substituted with one or more halogens or —CN.
C421. The compound of embodiment C420, wherein R⁵ is selected from —CF₂H, —CF₃, —CH₂CN, and —CH₂CH₃.
C422. The compound of embodiment C421, wherein R⁵ is —CF₃.
C423. The compound of any one of embodiments C387-C422, wherein the compound is a not a compound included in Table 4.
C424. A compound shown in Table 5, or a salt (e.g., pharmaceutically acceptable salt) thereof
C425. A compound shown in Table 6, or a salt (e.g., pharmaceutically acceptable salt) thereof.
C426. A compound shown in Table 7, or a salt (e.g., pharmaceutically acceptable salt) thereof. C427. A compound shown in Table 8, or a salt (e.g., pharmaceutically acceptable salt) thereof.
C428. A compound shown in Table 9, or a salt (e.g., pharmaceutically acceptable salt) thereof.
C429. A pharmaceutical composition comprising a compound of any one of embodiments C1-C428, or a salt (e.g., pharmaceutically acceptable salt) thereof and a pharmaceutically acceptable excipient.
C430. A compound of any one of embodiments C1-C428, or a salt (e.g., pharmaceutically acceptable salt) thereof, for use as a medicament.
C431. The compound of embodiment C430, wherein the medicament is useful in the prevention or treatment of a disease, disorder, or condition ameliorated by the inhibition of KRAS having a Q61H, G13D, G12D, G12V, G12C, G12S, G12A, or G12R mutation, or wild-type KRAS.
C432. The compound of embodiment C431, wherein the medicament is useful in the prevention or treatment of a disease, disorder, or condition ameliorated by the inhibition of wild-type KRAS.
C433. The compound of embodiment C431, wherein the medicament is useful in the prevention or treatment of a disease, disorder, or condition ameliorated by the inhibition of KRAS having a G12D, G12R, or G12V mutation.
C434. The compound of any one of embodiments C430-C432, wherein the medicament is useful in the prevention or treatment of a cancer.
C435. The compound of embodiment C434, wherein the cancer is selected from the group consisting of pancreatic cancer, colorectal cancer, and lung cancer.
C436. A compound of any one of embodiments C1-C428, or a salt (e.g., pharmaceutically acceptable salt) thereof, for use in the treatment of a disease, disorder, or condition.
C437. The compound of embodiment C436, wherein the disease, disorder, or condition is a cancer.
C438. The compound of embodiment C437, wherein the cancer is selected from the group consisting of pancreatic cancer, colorectal cancer, and lung cancer.
C439. The compound of any one of embodiments C436-C438, wherein the compound is used in the treatment of a disease, disorder, or condition in a subject in need thereof.
C440. A compound of any one of embodiments C1-C428, or a salt (e.g., pharmaceutically acceptable salt) thereof, for use in the manufacture of a medicament.
C441. The compound of embodiment C440, wherein the medicament is useful in the prevention or treatment of a disease, disorder, or condition ameliorated by the inhibition of KRAS having a Q61H, G13D, G12D, G12V, G12C, G12S, G12A, or G12R mutation, or wild-type KRAS.
C442. The compound of embodiment C441, wherein the medicament is useful in the prevention or treatment of a disease, disorder, or condition ameliorated by the inhibition of wild-type KRAS.
C443. The compound of embodiment C441, wherein the medicament is useful in the prevention or treatment of a disease, disorder, or condition ameliorated by the inhibition of KRAS having a G12D, G12R, or G12V mutation.
C444. The compound of any one of embodiments C440-C443, wherein the medicament is useful in the treatment of a cancer.
C445. The compound of embodiment C444, wherein the cancer is selected from the group consisting of pancreatic cancer, colorectal cancer, and lung cancer.
C446. A method, comprising administering a therapeutically effective amount of a compound of any one of embodiments C1-C428, or a salt (e.g., pharmaceutically acceptable salt) thereof, to a subject in need thereof.
C447. The method of embodiment C446, wherein the subject has a disease, disorder, or condition ameliorated by the inhibition of KRAS having a Q61H, G13D, G12D, G12V, G12C, G12S, G12A, or G12R mutation, or wild-type KRAS.
C448. The method of embodiment C447, wherein the disease, disorder, or condition is ameliorated by the inhibition of wild-type KRAS.
C449. The method of embodiment C447, wherein the disease, disorder, or condition is ameliorated by the inhibition of KRAS having a G12D, G12R, or G12V mutation.
C450. The method of any one of embodiments C446-C449, wherein the subject has a cancer.
C451. The method of embodiment C450, wherein the subject was previously diagnosed with the cancer.
C452. The method of embodiment C450, wherein the subject has previously undergone a treatment regimen for the cancer.
C453. The method of embodiment C451 or C452, wherein the subject has previously entered remission from the cancer.
C454. The method of any one of embodiments C450-C453, wherein the cancer is selected from the group consisting of pancreatic cancer, colorectal cancer, and lung cancer.
C455. The method of any one of embodiments C446-C454, wherein the compound, or the salt thereof, is administered in combination with an additional therapeutic agent.
C456. The use of a compound of any one of embodiments C1-C428, or a salt (e.g., pharmaceutically acceptable salt) thereof, for the manufacture of a medicament for the treatment of a cancer.
C457. The use of embodiment C456, wherein the cancer is selected from the group consisting of pancreatic cancer, colorectal cancer, and lung cancer.
C458. A method, comprising contacting a KRAS protein with a compound of any one of embodiments C1-C428, or a salt (e.g., pharmaceutically acceptable salt) thereof.
C459. The method of embodiment C458, wherein contacting the KRAS protein with the compound modulates KRAS.
C460. The method of embodiment C458 or C459, wherein the KRAS protein has a Q61H, G13D, G12D, G12V, G12C, G12S, G12A, or G12R mutation.
C461. The method of embodiment C458 or C459, wherein the KRAS protein is a wild-type KRAS protein.
C462. The method of any one of embodiments C458-C461, wherein the KRAS protein is in an active (GTP-bound) state.
C463. The method of any one of embodiments C458-C461, wherein the KRAS protein is in an inactive (GDP-bound) state.
C464. The method of any one of embodiments C458-C463, wherein the KRAS protein is located within a cell.
C465. The method of embodiment C464, wherein the cell is located within a subject.
C466. The method of embodiment C465, wherein the subject is a human.
C467. The method of embodiment C465 or C466, wherein the subject has a cancer.
C468. The method of embodiment C467, wherein the cancer is selected from the group consisting of pancreatic cancer, colorectal cancer, and lung cancer.
C469. A method of inhibiting the function of a wild-type KRAS protein or a KRAS protein having a Q61H, G13D, G12D, G12V, G12C, G12S, G12A, or G12R mutation, comprising contacting the KRAS protein with a compound of any one of embodiments C1-C428, or a salt (e.g., pharmaceutically acceptable salt) thereof.
C470. The method of embodiment C469, wherein the KRAS protein is a wild-type KRAS protein.
C471. The method of embodiment C469, wherein the KRAS protein has a Q61H, G13D, G12D, G12V, G12C, G12S, G12A, or G12R mutation.
C472. The method of embodiment C471, wherein the KRAS protein has a G12D, G12V, or G12R mutation.
C473. The method of any one of embodiments C469-C472, wherein the KRAS protein is in an active (GTP-bound) state.
C474. The method of any one of embodiments C469-C472, wherein the KRAS protein is in an inactive (GDP-bound) state.
C475. The method of any one of embodiments C469-C474, wherein the KRAS protein is located within a cell.
C476. The method of embodiment C475, wherein the cell is located within a subject.
C477. The method of embodiment C476, wherein the subject is a human.
C478. The method of embodiment C476 or C477, wherein the subject has a cancer.
C479. The method of embodiment C478, wherein the cancer is selected from the group consisting of pancreatic cancer, colorectal cancer, and lung cancer.
C480. A compound capable of inhibiting a wild-type KRAS protein or a KRAS protein having a Q61H, G13D, G12D, G12V, G12C, G12S, G12A, or G12R mutation in both its active (GTP-bound) and inactive (GDP-bound) state.
C481. The compound of embodiment C480, wherein the compound:
   (i) has IC₅₀≤0.1 μM, 0.1 μM<IC₅₀≤1 μM, 1 μM<IC₅₀≤10 μM, or 10 μM<IC₅₀ in the assay of Biological Example 1 (e.g., a protein:protein interaction (PPI) Homogenous Time Resolved Fluorescence (HTRF) analysis of 50 nM Avi-KRAS G12D (amino acids 1-169) GppNHp/RAF1 RBD-3×FLAG (52-151); 50 nM Avi-KRAS G12R (amino acids 1-169) GppNHp/RAF1 RBD-3×FLAG (52-151); 50 nM Avi-KRAS G12V (amino acids 1-169) GppNHp/RAF1 RBD-3×FLAG (52-151); 50 nM Avi-KRAS WT (amino acids 1-169) GppNHp/RAF1 RBD-3×FLAG (52-151); and/or 75 nM Avi-RAF1 RBD-3×FLAG; and/or
   (ii) has IC₅₀≤0.1 μM, 0.1 μM<IC₅₀≤1 μM, or IC₅₀>1 μM in the assay of Biological Example 2 (e.g., cell-based pERK HTRF assay in GP2d (G12D) and SW620 (G12V) cell).
C482. The compound of embodiment C481, wherein the compound:
   (i) has IC₅₀≤0.1 μM or 0.1 μM<IC₅₀≤1 μM in the assay of Biological Example 1 (e.g., a protein:protein interaction (PPI) Homogenous Time Resolved Fluorescence (HTRF) analysis of 50 nM Avi-KRAS G12D (amino acids 1-169) GppNHp/RAF1 RBD-3×FLAG (52-151): 50 nM Avi-KRAS G12R (amino acids 1-169) GppNHp/RAF1 RBD-3×FLAG (52-151): 50 nM Avi-KRAS G12V (amino acids 1-169) GppNHp/RAF1 RBD-3×FLAG (52-151): 50 nM Avi-KRAS WT (amino acids 1-169) GppNHp/RAF1 RBD-3×FLAG (52-151); and/or 75 nM Avi-RAF1 RBD-3×FLAG; and/or
   (ii) has IC₅₀≤0.1 μM or 0.1 μM<IC₅₀≤1 μM in the assay of Biological Example 2 (e.g., cell-based pERK HTRF assay in GP2d (G12D) and SW620 (G12V) cell).
C483. The compound of any one of embodiments C480-C482, wherein the compound is capable of irreversibly binding the KRAS protein.
C484. The compound of any one of embodiments C480-C482, wherein the compound is capable of reversibly binding the KRAS protein.
C485. The compound of any one of embodiments C480-C484, wherein the compound is a compound according to any one of embodiments C1-C428.

### EXAMPLES

Selected abbreviations used in the preceding sections and the Examples are summarized in Table

TABLE A-1
Abbreviations.

| Abbreviation | Term |
|---|---|
| MeCN | acetonitrile |
| cm | centimeter |
| ° C. | degrees Celsius |
| ° K | degrees Kelvin |
| BINAP | [1,1′-Binaphthalene]-2,2′-diyl)bis(diphenylphosphane) |
| DCM | dichloromethane |
| DIEA | N,N-diisopropylethylamine (Hünig's base) |
| DMF | dimethylformamide |
| DMSO | dimethyl sulfoxide |
| DPPA | diphenylphosphoryl azide |
| eq | equivalents |
| ESI | electrospray ion-mass spectrometry |
| EtOH | ethanol |
| EtOAc | ethyl acetate |
| g | gram |
| Hz | hertz |
| HATU | Hexafluorophosphate azabenzotriazole tetramethyl uronium |
| HPLC | high performance liquid chromatography |
| HTRF | homogenous time-resolved fluorescence |
| h | hour |
| kDa | kilodalton |
| LC | liquid chromatography |
| LCMS | liquid chromatography-mass spectrometry |
| L | liter |
| MS | mass spectra |
| MHz | megahertz |
| MeOH | methanol |
| MTBE | methyl tert-butyl ether |
| μg | microgram |
| μL | microliter |
| μM | micromolar |
| μm | micron |
| μs | microsecond |
| mg | milligram |
| mL | milliliter |
| mm | millimeter |
| mM | millimolar |
| mmol | millimole |
| min | minute |
| M | molar |
| nL | nanoliter |
| nm | nanometer |
| NCS | N-chlorosuccinimide |
| NMP | N-methyl-2-pyrrolidone |
| PE:EA | Petroleum ether:ethyl acetate |
| ppm | parts per million |
| PTLC | preparative thin layer chromatography |
| ¹HNMR | proton nuclear magnetic resonance |
| RBD | receptor binding domain |
| RP | reverse phase |
| rpm | revolutions per minute |
| SPR | surface plasmon resonance |
| THF | tetrahydrofuran |
| TMS | tetramethylsilane |
| TLC | thin layer chromatography |
| SOCl₂ | thionyl chloride |
| TEA | triethylamine |
| TFA | trifluoroacetic acid |
| TCEP | tris(2-carboxyethyl)phosphine |
| UV | ultraviolet |
| UV/Vis | ultraviolet/visible |

### Materials and Methods

Preparative thin layer chromatography (PTLC) separations described herein were typically performed on 20×20 cm plates (500-μm thick silica gel).

Chromatographic purifications were typically performed using Biotage Isolera One automated system running Biotage Isolera One 2.0.6 software (Biotage LLC, Charlotte, NC). Flow rates were the default values specified for the column in use. Reverse phase chromatography was performed using elution gradients of water and acetonitrile on KP-C18-HS Flash+ columns (Biotage LLC) of various sizes. Typical loading was between 1:50 and 1:1000 crude sample: RP SiO₂ by weight. Normal phase chromatography was performed using elution gradients of various solvents (e.g., hexane, ethyl acetate, methylene chloride, methanol, acetone, chloroform, MTBE, etc.). The columns were SNAP Cartridges containing KP-SIL or SNAP Ultra (25 pm spherical particles) of various sizes (Biotage LLC). Typical loading was between 1:10 to 1:150 crude sample: SiO₂ by weight. Alternatively, silica gel chromatography was performed on a Biotage Horizon flash chromatography system.

¹HNMR analyses of intermediates and exemplified compounds were typically performed on a Bruker Ascend TM 400 spectrometer (operating at 400 MHz), Bruker Ascend 700 MHz Advance Neo Spectrometer (Bruker-Biospin) or Bruker Advance ultrashield 300/54 (operating at 300 MHz) at 298° K following standard operating procedure suggested by manufacturer. Reference frequency was set using TMS as an internal standard. Chemical shift values (8) are reported in parts per million (ppm) with splitting patterns abbreviated to: s (singlet), br, s (broad singlet), d (doublet), dd (double doublet), t (triplet), and m (multiplet). The coupling constant (J) is given in Hz. Typical deuterated solvents were utilized as indicated in the individual examples.

LCMS analysis were typically performed using one of the following conditions:

(1) LCMS spectra were taken on an Agilent Technologies 6120B Quadrupole spectrometer. The mobile phase for the LC was acetonitrile (A) with 0.1% formic acid, and water (B) with 0.1% formic acid, and the eluent gradient was from 5-95% A in 6.0 min, 5%-40% A in 6.0 min, 80-100% A in 6.0 min using a poroshell 120 EC-C18 50 mm×3.0 mm×2.7 μm capillary column: Flow Rate: 0.7 mL/min. Mass spectra (MS) were measured by electrospray ion-mass spectroscopy (ESI). All temperatures are in degrees Celsius (° C.) unless otherwise noted.

(2) LCMS spectra were taken on an Agilent Technologies 1290-6420 Triple Quadrupole spectrometer. The mobile phase for the LC was acetonitrile (A) with 0.05% formic acid, and water (B) with 0.05% formic acid, and the eluent gradient was from 5-95% A in 5.0 min, using a ZORBAX SB-C18 50 mm×2.1 mm×1.8 μm capillary column: Flow Rate: 0.3 mL/min. Mass spectra (MS) were measured by electrospray ion-mass spectroscopy (ESI). All temperatures are in degrees Celsius unless otherwise noted.

(3) LC-MS analysis was performed using an Agilent 6120b single quadrupole mass spectrometer with an Agilent 1260 infinity II chromatography separations module and Agilent 1260 infinity II photodiode array detector controlled by Agilent Chemstation software. The HPLC column used was an Agilent ZORBAX Eclipse XDB-C18 4.6 mm×150 mm×3.5 μm RapidResol column with a mobile phase of water (0.1% formic acid)/MeCN (0.1% formic acid) and a gradient of 5-95% MeCN over 10 minutes at a flow rate of 1 mL/min. Accurate mass data was obtained using a Thermo Fisher extractive plus EMR orbitrap LCMS system. Exact mass values were calculated by ChemCalc.

(4) LCMS spectra were taken on an alliance Waters 2695 coupled to a dual absorbance detector waters 2487 and a waters micro mass ZQ-2000 single quadrupole spectrometer. The mobile phase for the LC was acetonitrile (A) and water (B) with 0.01% formic acid, and the eluent gradient was from 5-100% A in 10.0 minute using a Kromasil 100-5-C18 150 mm×4.6 mm×5 μm column. Mass spectra (MS) were measured by electrospray ion-mass spectroscopy (ESI). All temperatures are in degrees Celsius unless otherwise noted.

Typically, analytical HPLC mass spectrometry conditions were as follows:

LC1: Agilent Technologies 1260 Infinity coupled, Column: poroshell 120 EC-C18 150 mm×4.6 mm×4 μm; Temperature: 40° C.; Eluent: 5:95 v/v acetonitrile/water+0.02% trifluoroacetic acid in 20 min; Flow Rate: 1.2 ml/min: Detection: VWD, 190-600 nm.

LC2: C₁₈-Reverse phase preparative HPLC was performed using a Waters purification system with 2489 UV/Vis detector, 2545 Gradient module, and Fraction collector III controlled by Waters Chromescope v1.6. The preparative HPLC column used was a Waters XBridge® Prep C18 5 μm OBD™ 19×250 mm column with a mobile phase of water/MeCN or water (0.1% TFA)/MeCN (0.1% TFA).

Preparative HPLC were carried out with one of the following two conditions:

Condition 1: GILSON Preparative HPLC System; Column: Ultimate XB—C18, 21.2 mm×250 mm, 5 μm; Mobile phase: Water with 0.1% trifluoroacetic acid; MeCN with 0.1% trifluoroacetic acid; Method: 15 minutes gradient elution; Initial organic: 10% to 30%; Final organic: 60% to 80%; UV1: 240; UV2: 230; Flow: 15 mL/min.

Condition 2: C18-Reverse phase preparative HPLC was performed using a Waters purification system with 2489 UV/Vis detector, 2545 Gradient module, and Fraction collector III controlled by Waters Chromoscope v1.6. The preparative HPLC column used was a Waters XBridge® Prep C18 5 μm OBD™ 19×250 mm column with a mobile phase of water/MeCN or water (0.1% TFA)/MeCN (0.1% TFA).

Compound names were generated with ChemDraw Professional.

The compounds provided herein, including in various forms such as salts, esters, tautomers, prodrugs, zwitterionic forms, stereoisomers, etc., may be prepared according to various methods including those set forth in the following examples.

### Synthetic Example 1: Synthesis of (1R,3S)-3-(((S)-7-(2-amino-7-fluorobenzo[d]thiazol-4-yl)-8-fluoro-2-((tetrahydro-1H-pyrrolizin-7a(5H)-yl)methoxy)-6-(trifluoromethyl)quinazolin-4-yl)(methyl)amino)spiro[3.4]octan-1-ol (Compound 10) and (1R,3S)-3-(((R)-7-(2-amino-7-fluorobenzo[d]thiazol-4-yl)-8-fluoro-2-((tetrahydro-1H-pyrrolizin-7a(5H)-yl)methoxy)-6-(trifluoromethyl)quinazolin-4-yl)(methyl)amino)spiro[3.4]octan-1-ol (Compound 11)

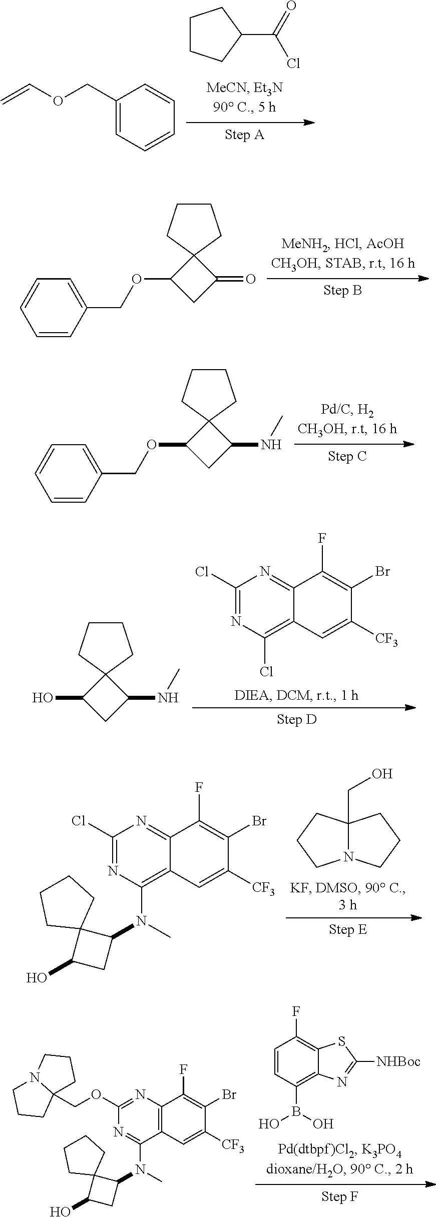

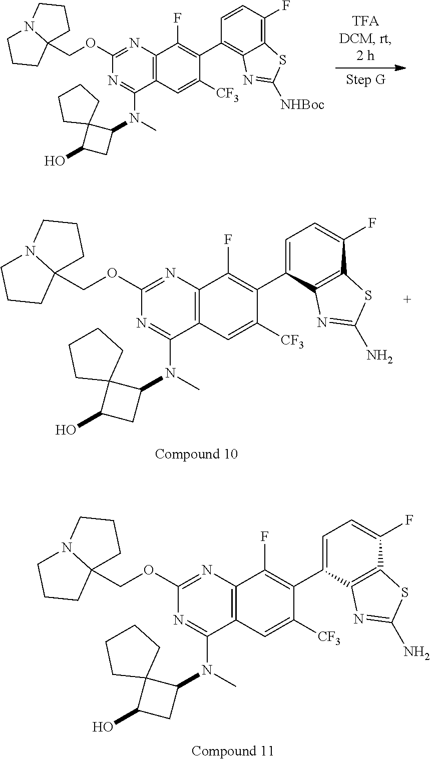

Step A: Preparation of 1-benzyloxyspiro[3.4]octan-3-one: To a mixture of vinyloxmethyl benzene (4.05 g, 30.2 mmol, 2.00 eq) in MeCN (10 mL) was added DIEA (3.2 mL, 18.1 mmol, 1.20 eq) and stirred for 10 mins. Then cyclopentanecarbonyl chloride (2.00 g, 15.1 mmol, 1.00 eq) in MeCN (5.0 mL) was added. After addition, the reaction was stirred at 90° C. for 3 hs. The reaction was concentrated to dryness and the residue was taken up in EtOAc (50 mL×2). The organic layers were washed with water (30 mL×2) and brine (30 mL×2), and dried (MgSO₄) before concentration to dryness. The crude was then purified by flash column chromatography (eluting with 10% EtOAc in hexanes). The desired fractions were collectd and concentrated to give 1-benzyloxyspiro[3.4]octan-3-one (800 mg, 3.47 mmol, 23% yield). LCMS: (ES+): m/z 231, [M+H].

Step B: Preparation of (1,3-cis)-3-benzyloxy-N-methyl-spiro[3.4]octan-1-amine: To a mixture of 1-benzyloxyspiro[3.4]octan-3-one (300 mg, 1.30 mmol, 1.00 eq) in DCM (5.0 mL) was added MeNH₂·HCl (176 mg, 2.61 mmol, 1.00 eq), followed by NaOAc (214 mg, 2.61 mmol, 2.00 eq) and AcOH (156 mg, 2.61 mmol, 2.00 eq). The reaction was stirred at rt for 16 hrs under N₂. To the mixture was added STAB (552 mg, 2.61 mmol, 2.00 eq) and stirred for 30 mins. The reaction was concentrated to dryness and the residue was taken up in EtOAc (30 mL×2) and the organic layers were washed with water (30 mL×2) and brine (30 mL×2), and dried (MgSO₄) before concentration to dryness. The crude was then purified by flash column chromatography (eluting with 5% MeOH in DCM). The desired fractions were collected and concentrated to give (1,3-cis)-3-benzyloxy-N-methyl-spiro[3.4]octan-1-amine (169 mg, 0.689 mmol, 53% yield). LCMS: (ES+): m/z 246, [M+H].

Step C: Preparation of (1,3-cis)-1-(methylamino)spiro[3.4]octan-3-ol: To a mixture of (1,3-cis)-3-benzyloxy-N-methyl-spiro[3,4]octan-1-amine (150 mg, 0.477 mmol, 1.00 eq) in methanol (5.0 mL) was added Pd/C (10%, 40 mg) and stirred at rt under N₂ for 16 hrs; TLC and LCMS showed the reaction was complete. The reaction mixture was filtered and concentrated to give (1,3-cis)-1-(methylamino)spiro[3.4]octan-3-ol (104 mg, 0.670 mmol, 140% yield) as yellow oil. LCMS: (ES+): m/z 156.2, [M+H].

Step D: Preparation of(1,3-cis)-3-((7-bromo-2-chloro-8-fluoro-6-(trifluoromethyl)quinazolin-4-yl)(methyl)amino)spiro[3,4]octan-1-ol: To a mixture of 7-bromo-2,4-dichloro-8-fluoro-6-(trifluoromethyl)quinazoline (100 mg, 0.275 mmol, 1.00 eq) in DCM (2.0 mL) was added (1,3-cis)-1-(methylamino)spiro[3.4]octan-3-ol (64 mg, 0.412 mmol, 1.50 eq) and DIEA (0.20 mL, 1.10 mmol, 4.00 eq) at 0° C. The reaction was stirred at rt for 1 hr. LCMS showed the starting material consumed completely. The reaction was concentrated to dryness and the residue was taken up in EtOAc (30 mL×2). The organic layers were washed with water (30 mL×2) and brine (30 mL), and dried (MgSO₄) before concentration to dryness. The crude was then purified by prep-TLC (eluting with 25% EtOAc in hexanes) to give (1,3-cis)-3-((7-bromo-2-chloro-8-fluoro-6-(trifluoromethyl)quinazolin-4-yl)(methyl)amino)spiro[3.4]octan-1-ol(70 mg, 0.145 mmol, 53% yield). LCMS: (ES+): m/z 482.0, [M+H].

Step E: Preparation of (1,3-cis)-3-((7-bromo-2-chloro-8-fluoro-6-(trifluoromethyl)quinazolin-4-yl)(methyl)amino)spiro[3.4]octan-1-ol: A mixture of 1,2,3,5,6,7-hexahydropyrrolizin-8-ylmethanol (106 mg, 0.749 mmol, 1.20 eq). (1,3-cis)-3-((7-bromo-2-chloro-8-fluoro-6-(trifluoromethyl)quinazolin-4-yl)(methyl)amino)spiro[3.4]octan-1-ol (70 mg, 0.145 mmol, 1.00 eq) and KF (179 mg, 8.00 eq, 3.08 mmol) in DMSO (1.5 mL) was stirred at 90° C. under Ar for 3 hrs. The reaction was qucnchcd by water and taken up in EtOAc (30 mL×3). The organic layers were washed with water (20 mL×2) and brine (20 mL×1), and dried (MgSO₄) before concentration to dryness. The residue was then purified by Prep-TLC (EtOAc) to give (1,3-cis)-3-((7-bromo-2-chloro-8-fluoro-6-(trifluoromethyl)quinazolin-4-yl)methyl)amino)spiro[3.4]octan-1-01 (35 mg, 0.0597 mmol, 41% yield).

Step F: Preparation of tert-butyl (7-fluoro-4-(8-fluoro-4-(((1,3-cis)-3-hydroxyspiro[3.4]octan-1-yl)methyl)amino)-2-((tetrahydro-1H-pyrrolizin-7a(5H)-yl)methoxy)-6-(trifluoromethyl) quinazolin-7-yl)benzo[d]thiazol-2-yl)carbamate: A mixture of (1,3-cis)-3-((7-bromo-2-chloro-8-fluoro-6-(trifluoromethyl)quinazolin-4-yl)(methyl)amino)spiro[3.4]octan-1-ol (30 mg, 1.00 eq, 0.0511 mmol), (2-((tert-butoxycarbonyl)amino)-7-fluorobenzo[d]thiazol-4-yl)boronic acid (19 mg, 1.20 eq, 0.0596 mmol), Pd(dtppf)Cl₂ (3.1 mg, 0.0800 eq, 0.00477 mmol), and K₃PO₄ (51 mg, 4.00 eq, 0.238 mmol) in 1,4-dioxane/H₂O ((1.0 mL/0.30 mL) was stirred at 90° C. under Ar for 2 hrs. The reaction was concentrated to dryness and the residue was purified by prep-TLC (50% EtOAc in hexanes to 100% EtOAc) to give tert-butyl (7-fluoro-4-(8-fluoro-4-(((1,3-cis)-3-hydroxyspiro[3.4]octan-1-yl)(methyl)amino)-2-((tetrahydro-1H-pyrrolizin-7a(5H)-yl)methoxy)-6-(trifluoromethyl)quinazolin-7-yl)benzo[d]thiazol-2-yl)carbamate (33 mg, 0.0425 mmol, 83% yield).

Step G: Preparation of (1R,3S)-3-(((S)-7-(2-amino-7-fluorobenzo[d]thiazol-4-yl)-8-fluoro-2-((tetrahydro-1H-pyrrolizin-7a(5H)-yl)methoxy)-6-(trifluoromethyl)quinazolin-4-yl)(methyl)amino)spiro[3.4]octan-1-ol and (1R,3S)-3-(((R)-7-(2-amino-7-fluorobenzo[d]thiazol-4-yl)-8-fluoro-2-((tetrahydro-1H-pyrrolizin-7a(5H)-yl)methoxy)-6-(trifluoromethyl)quinazolin-4-yl)(methyl)amino)spiro[3.4]octan-1-ol: To a solution of tert-butyl (7-fluoro-4-(8-fluoro-4-(((1,3-cis)-3-hydroxyspiro[3.4]octan-1-yl)(methyl)amino)-2-((tetrahydro-1H-pyrrolizin-7a(5H)-yl)methoxy)-6-(trifluoromethyl)quinazolin-7-yl)benzo[d]thiazol-2-yl)carbamate (9.0 mg, 0.0118 mmol, 1.00 eq) in DCM (1 mL) was added TFA (0.50 mL) at 25° C. The mixture was stirred at room temperature for 2 hrs. The mixture was concentrated and the residue was purified by Prep-HPLC to give (1R,3S)-3-(((S)-7-(2-amino-7-fluorobenzo[d]thiazol-4-yl)-8-fluoro-2-((tetrahydro-1H-pyrrolizin-7a(5H)-yl)methoxy)-6-(trifluoromethyl)quinazolin-4-yl)(methyl)amino)spiro[3.4]octan-1-ol (2.41 mg, 0.00357 mmol, 30% yield), as the first peak: LCMS: (ES+): m/z 675.3, [M+H]. ¹H NMR (400 MHz, CD₃OD): δ 8.35 (s, 1H), 7.25-7.20 (m, 1H), 7.00 (t, J=8.8 Hz, 1H), 4.80-4.70 (m, 1H), 4.63-4.58 (m, 1H), 4.55-4.46 (m, 1H), 3.90-3.85 (m, 1H), 3.70-3.60 (m, 2H), 3.55 (s, 3H), 3.30-3.25 (m, 2H), 2.75-2.68 (m, 1H), 2.68-2.60 (m, 1H), 2.40-2.00 (m, 8H), 1.81-1.71 (m, 1H), 1.70-1.20 (m, 7H); and (1R,3S)-3-(((R)-7-(2-amino-7-fluorobenzo[d]thiazol-4-yl)-8-fluoro-2-((tetrahydro-1H-pyrrolizin-7a(5H)-yl)methoxy)-6-(trifluoromethyl)quinazolin-4-yl)(methyl)amino)spiro[3.4]octan-1-ol (2.17 mg, 0.00322 mmol, 27% yield), as the second peak: LCMS: (ES+): m/z 675.3, [M+H]. ¹H NMR (400 MHz, CD₃OD): δ8.34 (s, 1H), 7.25-7.20 (m, 1H), 7.00 (t, J=8.8 Hz, 1H), 4.80-4.70 (m, 1H), 4.63-4.58 (m, 1H), 4.55-4.46 (m, 1H), 3.90-3.85 (m, 1H), 3.70-3.60 (m, 2H), 3.55 (s, 3H), 3.30-3.25 (m, 2H), 2.75-2.68 (m, 1H), 2.66-2.58 (m, 1H), 2.40-2.00 (m, 8H), 1.81-1.71 (m, 1H), 1.70-1.20 (m, 7H).

### Synthetic Example 2: Synthesis of 2-amino-7-fluoro-4-(8-fluoro-2-(((2R,7aS)-2-fluorotetrahydro-1H-pyrrolizin-7a(5H)-yl)methoxy)-4-(((1s,3R)-3-hydroxycyclobutyl)(methyl)amino)-6-(trifluoromethyl)quinazolin-7-yl)benzo[b]thiophene-3-carbonitrile (Compound 18), 2-amino-7-fluoro-4-((S)-8-fluoro-2-(((2R,7aS)-2-fluorotetrahydro-1H-pyrrolizin-7a(5H)-yl)methoxy)-4-(((1s,3R)-3-hydroxycyclobutyl)(methyl)amino)-6-(trifluoromethyl)quinazolin-7-yl)benzo[b]thiophene-3-carbonitrile (Compound 19), and 2-amino-7-fluoro-4-((R)-8-fluoro-2-(((2R,7aS)-2-fluorotetrahydro-1H-pyrrolizin-7a(5H)-yl)methoxy)-4-(((1s,3S)-3-hydroxycyclobutyl)((methyl)amino)-6-(trifluoromethyl)quinazolin-7-yl)benzo[b]thiophene-3-carbonitrile (Compound 20)

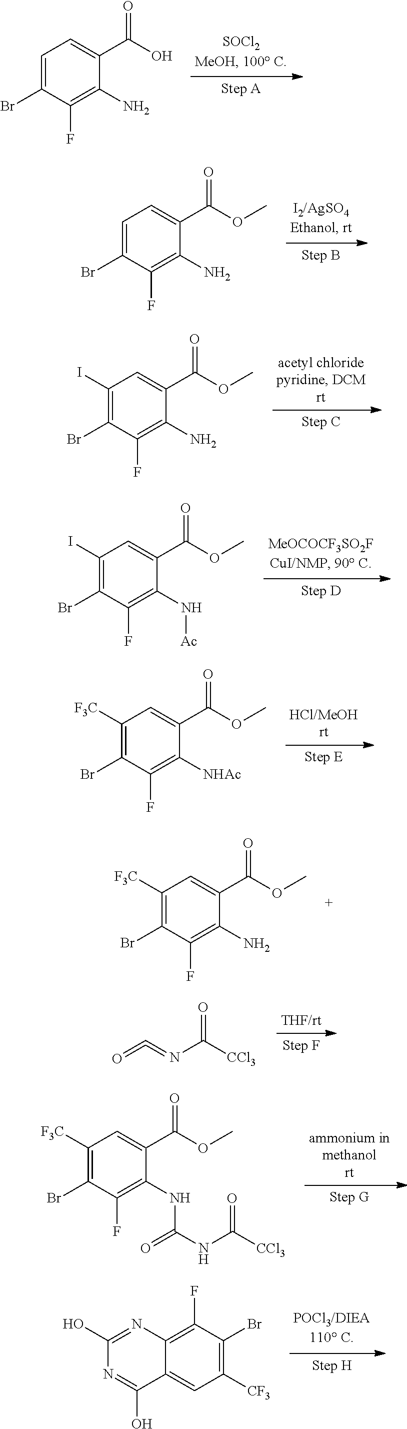

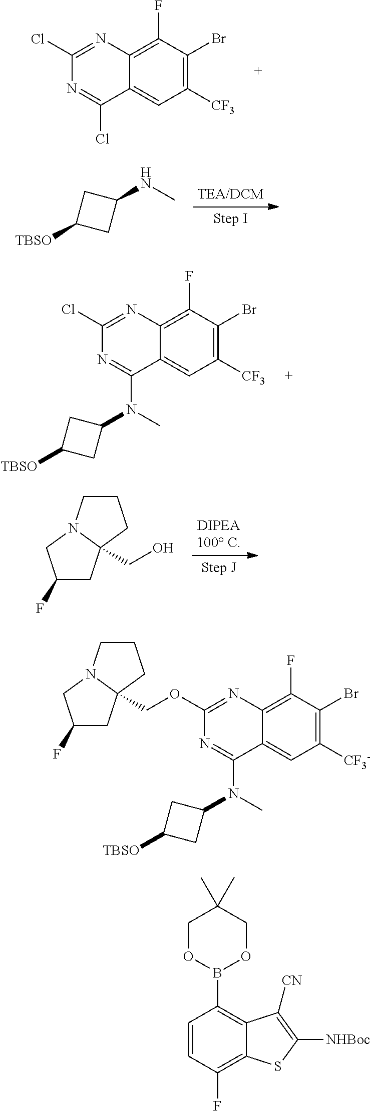

Step A: Preparation of methyl 2-amino-4-bromo-3-fluorobenzoate: To a stirring solution of 2-amino-4-bromo-3-fluorobenzoic acid (5.0 g, 21.4 mmol) in MeOH (30 mL) was added dropwise thionyl chloride (15.6 mL, 21 mmol) at 0° C. under argon. The resulting mixture was heated to 100° C. for 16 hours. The solvent was evaporated, and the residue was dissolved in ethyl acetate (100 mL). The organic layer was washed with a saturated aqueous NaHCO₃ solution, then dried over Na₂SO₄, filtered, and concentrated under vacuum. The resulting crude material was purified by silica gel column chromatography using EtOAc in hexanes (0/o to 20%) as eluent to give methyl 2-amino-4-bromo-3-fluorobenzoate (5.0 g, 94%) as a solid. LCMS ESI (+) m/z 249 (M+H). ¹HNMR (300 MHz, CDCl₃) δ 7.53 (dd, J=8.8, 1.8 Hz, 1H), 6.78 (dd, J=8.8, 6.3 Hz, 1H), 5.93 (s, 1H), 3.90 (s, 1H).

Step B: Preparation of methyl 2-amino-4-bromo-3-fluoro-5-iodobenzoate: To a mixture of iodine (7.16 g, 28 mmol) and silver sulfate (5.3 g, 17 mmol) in EtOH (200 mL), methyl 2-amino-4-bromo-3-fluorobenzoate (5.0 g, 20 mmol) was added and the resulting mixture was stirred at ambient temperature for 45 minutes. The solid was filtered off and washed with DCM, and the filtrate was concentrated under vacuum. The residue was dissolved in DCM and washed with 10% sodium thiosulphate solution, followed by brine, and the resulting organic solution was dried over Na₂SO₄, filtered and concentrated under vacuum to give methyl 2-amino-4-bromo-3-fluoro-5-iodobenzoate (6.66 g, 88% yield) as a yellow solid. LCMS ESI (+) m/z 373 (M+H). ¹HNMR (300 MHz, CDCl₃) δ 8.14 (d, J=1.9 Hz, 1H), 5.94 (s, 2H), 3.91 (s, 3H).

Step C: Preparation of methyl 2-acetamido-4-bromo-3-fluoro-5-iodobenzoate: Methyl 2-amino-4-bromo-3-fluoro-5-iodobenzoate (3.50 g, 9.4 mmol) and pyridine (2.3 ml, 28 mmol) were dissolved in DCM at 0° C. Acetyl chloride (0.79 ml, 11 mmol) was added and the reaction was warmed to ambient temperature and stirred at this temperature for 16 hours. The reaction mixture was concentrated under vacuum and the residue obtained was purified by silica gel column chromatography using ethyl acetate in hexanes (0% to 30%) as eluent to give methyl 2-acetamido-4-bromo-3-fluoro-5-iodobenzoate (2.7 g, 69%) as a solid. LCMS ESI (+) m/z 417 (M+H). ¹HNMR (300 MHz, CDCl₃) δ 8.87 (s, 1H), 8.25 (s, 1H), 3.95 (s, 3H), 2.26 (s, 3H).

Step D: Preparation of methyl 2-acetamido-4-bromo-3-fluoro-5-(trifluoromethyl)benzoate: To a stirred solution of methyl 4-bromo-2-acetamido-3-fluoro-5-iodobenzoate (1.0 g, 2.4 mmol) and methyl fluorosulfonyldifluoroacetate (0.92 g, 0.72 mmol) in NMP (22.0 mL) at ambient temperature, CuI (0.14 g, 0.73 mmol) was added and the resulting mixture was stirred at 80° C. for 16 hours. Once cooled to ambient temperature, the mixture was quenched with water and extracted with ethyl acetate. The organic layer was dried over Na₂SO₄, filtered, and concentrated, and the crude material was purified by silica gel column chromatography using ethyl acetate in hexanes (0% to 20%) as eluent to give methyl 2-acetamido-4-bromo-3-fluoro-5-(trifluoromethyl)benzoate (0.64 g, 74%) as solid. LCMS ESI (+) m/z 358 (M+H) ¹HNMR (300 MHz, CDCl₃) δ 9.23 (s, 1H), 8.10 (s, 1H), 3.93 (s, 3H), 2.28 (s, 3H).

Step E: Preparation of 2-amino-4-bromo-3-fluoro-5-(trifluoromethyl)benzoate: A mixture of methyl 2-acetamido-4-bromo-3-fluoro-5-(trifluoromethyl)benzoat (1.20 g, 3.35 mmol) in 3 M HCl in MeOH was heated at 60° C. for 2 hours. Once cooled to ambient temperature, the solvent was evaporated, and the crude product was partitioned between EtOAc and saturated NaHCO₃. The organic layer was separated, dried over sodium sulfate, filtered, and evaporated to provide 2-amino-4-bromo-3-fluoro-5-(trifluoromethyl)benzoate (1.00 g, 94%) as an oil. LCMS ESI (+) m/z 316.9 (M+H). ¹H NMR (300 MHz, CDCl₃) δ 8.01 (s, 1H), 6.28 (s, 2H), 3.94 (s, 3H).

Step F: Preparation of methyl 4-bromo-3-fluoro-2-(3-(2,2,2-trichloroacetyl)ureido)-5-(trifluoromethyl)benzoate: To a mixture of methyl 2-amino-4-bromo-3-fluoro-5-(trifluoromethyl)benzoate (0.80 g, 2.53 mmol) in THF (4.2 mL) was added trichloroethanecarbonyl isocyanate (0.45 mL, 3.79 mmol) at ambient temperature. After 15 minutes, the reaction mixture was evaporated, followed by the addition of MTBE and the solid formed was collected and washed with MTBE to provide methyl 4-bromo-3-fluoro-2-(3-(2,2,2-trichloroacetyl)ureido)-5-(trifluoromethyl)benzoate (0.71 g, 56%). LCMS ESI (+) m/z 529.99 (M+Na). ¹H NMR (300 MHz, CDCl₃) δ 10.91 (s, 1H), 8.65 (s, 1H), 8.23 (s, 1H), 4.03 (s, 3H).

Step G: Preparation of 7-bromo-8-fluoro-6-(trifluoromethyl)quinazoline-2,4-diol: To a solution of methyl 4-bromo-3-fluoro-2-(3-(2,2,2-trichloroacetyl)ureido)-5-(trifluoromethyl)benzoate (0.71 g, 1.40 mmol) in methanol (7.0 mL) was added a 7 M solution of ammonia in methanol (0.46 mL, 3.23 mmol) at ambient temperature and stirred at ambient temperature for 1 hour. The mixture was concentrated under reduced pressure to provide a solid which was co-evaporated with ether to provide 7-bromo-8-fluoro-6-(trifluoromethyl)quinazoline-2,4-diol (0.60 g, 100%) as solid LCMS ESI (+) m/z 260.0 (M+H). ¹H NMR (300 MHz, DMSO-d₆) δ 8.41 (s, 1H), 8.28 (s, 1H), 7.99 (s, 1H).

Step H: Preparation of 7-bromo-2,4-dichloro-8-fluoro-6-(trifluoromethyl)quinazoline: To a stirring solution of phosphorus oxychloride (0.97 mL, 10.5 mmol) and Hunig's base (0.40 mL, 2.29 mmol) was added 7-bromo-8-fluoro-6-(trifluoromethyl)quinazoline-2,4-diol (0.15 g, 0.46 mmol) at 0° C. After addition, the resulting mixture was stirred at 110° C. for 1 hour. Once cooled down to ambient temperature, the mixture was evaporated and co-evaporated with chloroform to give 7-bromo-2,4-dichloro-8-fluoro-6-(trifluoromethyl)quinazoline which used as such for the next step. ¹H NMR (300 MHz, CDCl₃) δ 8.45 (s, 1H).

Step I: Preparation of 7-bromo-N-((1s,3s)-3-((tert-butyldimethylsilyl)oxy)cyclobutyl)-2-chloro-8-fluoro-N-methyl-6-(trifluoromethyl)quinazolin-4-amine: To a mixture of 7-bromo-2,4-dichloro-8-fluoro-6-(trifluoromethyl)quinazoline (1.00 eq, 6 g, 16.57 mmol) in DCM (60 mL) at −40° C. was added TEA (3.00 eq, 5.02 g, 49.72 mmol), followed by (1s,3s)-3-((tert-butyldimethylsilyl)oxy)-N-methylcyclobutan-1-amine (1.00 eq, 3.56 g, 16.57 mmol). The resulting mixture was extracted with DCM (40 mL×3). The combined organic layers were washed with brine (20 mL), dried over anhydrous sodium sulfate, filtered and concentrated under reduced pressure. The crude was purified by column chromatography eluted with PE/EA (100:1-50:1) to obtain 7-bromo-N-((1s,3s)-3-((tert-butyldimethylsilyl)oxy)cyclobutyl)-2-chloro-8-fluoro-N-methyl-6-(trifluoromethyl)quinazolin-4-amine (6 g, 11.09 mmol, 66.93% yield) as a yellow oil. LCMS ESI (+) m/z 542.1 (M+H).

Step J: Preparation of 7-bromo-N-((1s,3R)-3-((tert-butyldimethylsilyl)oxy)cyclobutyl)-8-fluoro-2-(((2R,7aS)-2-fluorotetrahydro-1H-pyrrolizin-7a(5H)-yl)methoxy)-N-methyl-6-(trifluoromethyl)quinazolin-4-amine: To a mixture of 7-bromo-N-((1s,3s)-3-((tert-butyldimethylsilyl)oxy)cyclobutyl)-2-chloro-8-fluoro-N-methyl-6-(trifluoromethyl)quinazolin-4-amine (1.00 eq, 6 g, 11.09 mmol) and ((2R,7aS)-2-fluorotetrahydro-1H-pyrrolizin-7a(5H)-yl)methanol (2.00 eq, 3.53 g, 22.18 mmol) in DIPEA (50 mL) at ambient temperature. The reaction mixture was stirred at 100° C. for 6 hours. The reaction mixture was cooled to room temperature, diluted with water and extracted with EtOAc (50 mL×3). The organic layer was washed with brine (50 mL), dried over anhydrous sodium sulfate, filtered and concentrated. The crude product was purified by column chromatography eluted with PE/EA (10:1-3:1) to obtain 7-bromo-N-((1s,3R)-3-((tert-butyldimethylsilyl)oxy)cyclobutyl)-8-fluoro-2-(((2R,7aS)-2-fluorotetrahydro-1H-pyrrolizin-7a(5H)-yl)methoxy)-N-methyl-6-(trifluoromethyl)quinazolin-4-amine (4 g, 6.02 mmol, 54.30% yield) as a yellow oil. LCMS ESI (+) m/z 665.3 (M+H).

Step K: Preparation of tert-butyl (4-(4-(((1s,3R)-3-((tert-butyldimethylsilyl)oxy)cyclobutyl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-3-cyano-7-fluorobenzo[b]thiophen-2-yl)carbamate: To a stirring suspension of 7-bromo-N-((1s,3R)-3-((tert-butyldimethylsilyl)oxy)cyclobutyl)-8-fluoro-2-(((2R,7aS)-2-fluorotetrahydro-1H-pyrrolizin-7a(5H)-yl)methoxy)-N-methyl-6-(trifluoromethyl)quinazolin-4-amine (1.00 eq, 2.00 g, 3.01 mmol) in 1,4-dioxane (20 mL) was added Cs₂CO₃ (3.00 eq, 2.94 g, 9.03 mmol), tert-butyl (3-cyano-4-(5,5-dimethyl-1,3,2-dioxaborinan-2-yl)-7-fluorobenzo[b]thiophen-2-yl)carbamate (1.50 eq, 1.83 g, 4.51 mmol) and bis(diphenylphosphinophenyl)ether palladium (II)dichloride (0.15 eq, 350.1 mg, 0.45 mmol). The reaction mixture was stirred at 90° C. under N₂ for 3 h. The reaction mixture was cooled to room temperature, diluted with water and extracted with EtOAc (20 mL×3). The organic layer was washed with brine (30 mL), dried over anhydrous sodium sulfate, filtered and concentrated. The crude product was purified by column chromatography eluted with PE/EA (5:1-2:1) to obtain tert-butyl (4-(4-(((1s,3R)-3-((tert-butyldimethylsilyl)oxy)cyclobutyl(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-3-cyano-7-fluorobenzo[b]thiophen-2-yl)carbamate (1000 mg, 1.14 mmol, 37.91% yield) as a yellow solid. LCMS ESI (+) m/z 877.2 (M+H).

Step L: Preparation of tert-butyl (3-cyano-7-fluoro-4-(8-fluoro-2-(((2R,7aS)-2-fluorotetrahydro-1H-pyrrolizin-7a(5H)-yl)methoxy)-4-(((1s,3R)-3-hydroxycyclobutyl)(methyl)amino)-6-(trifluoromethyl)quinazolin-7-yl)benzo[b]thiophen-2-yl)carbamate: To a solution of tert-butyl (4-(4-(((1s,3R)-3-((tert-butyldimethylsilyl)oxy)cyclobutyl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-3-cyano-7-fluorobenzo[b]thiophen-2-yl)carbamate (1.00 eq. 1000 mg, 1.14 mmol) in THF (10 mL) was added 1M TBAF (5.00 eq, 5.7 mL, 5.70 mmol) and the reaction was stirred for 2 hours at room temperature. The reaction mixture was diluted with water (20 mL) at 0° C., and extracted with EA (20 mL×3). The combined organic layers were washed with brine (20 mL), dried over anhydrous sodium sulfate, filtered and concentrated under reduced pressure. The residue was purified by prep-TLC (DCM:MeOH=10:1) to give tert-butyl (3-cyano-7-fluoro-4-(8-fluoro-2-(((2R,7aS)-2-fluorotetrahydro-1H-pyrrolizin-7a(5H)-yl)methoxy)-4-(((1s,3R)-3-hydroxycyclobutyl)(methyl)amino)-6-(trifluoromethyl)quinazolin-7-yl)benzo[b]thiophen-2-yl)carbamate (600 mg, 0.787 mmol, 69.05% yield) as a yellow solid. LCMS ESI(+) m/z 673.1 (M+1).

Step M: Preparation of 2-amino-7-fluoro-4-(8-fluoro-2-(((2R,7aS)-2-fluorotetrahydro-1H-pyrrolizin-7a(5H)-yl)methoxy)-4-(((1s,3R)-3-hydroxycyclobutyl)(methyl)amino)-6-(trifluoromethyl)quinazolin-7-yl)benzo[b]thiophene-3-carbonitrile (Compound 18), 2-amino-7-fluoro-4-((S)-8-fluoro-2-(((2R,7aS)-2-fluorotetrahydro-1H-pyrrolizin-7a(5H)-yl)methoxy)-4-(((1s,3R)-3-hydroxycyclobutyl)(methyl)amino)-6-(trifluoromethyl)quinazolin-7-yl)benzo[b]thiophene-3-carbonitrile (Compound 19), and 2-amino-7-fluoro-4-((R)-8-fluoro-2-(((2R,7aS)-2-fluorotetrahydro-1H-pyrrolizin-7a(5H)-yl)methoxy)-4-(((1s,3S)-3-hydroxycyclobutyl)methyl)amino)-6-(trifluoromethyl)quinazolin-7-yl)benzo[b]thiophene-3-carbonitrile (Compound 20): To a solution of tert-butyl (3-cyano-7-fluoro-4-(8-fluoro-2-(((2R,7aS)-2-fluorotetrahydro-1H-pyrrolizin-7a(5H)-yl)methoxy)-4-(((1s,3R)-3-hydroxycyclobutyl)(methyl)amino)-6-(trifluoromethyl)quinazolin-7-yl)benzo[b]thiophen-2-yl)carbamate (1.00 eq, 600 mg, 0.787 mmol) in DCM (6 mL) was added TFA (34.05 eq, 2.0 mL, 26.8 mmol) and the reaction was stirred for 2 hours at room temperature. The mixture was neutralized with NaHCO₃ aqueous solution to pH=8 at 0° C., and extracted with DCM (3×5 mL). The organic layer was washed with water and brine, dried over Na₂SO₄ and concentrated in vacuo. Then the residue was purified by prep-HPLC and dried by lyophilization to give 2-amino-7-fluoro-4-(8-fluoro-2-(((2R,7aS)-2-fluorotetrahydro-1H-pyrrolizin-7a(5H)-yl)methoxy)-4-(((1s,3R)-3-hydroxycyclobutyl)((methyl)amino)-6-(trifluoromethyl)quinazolin-7-yl)benzo[b]thiophene-3-carbonitrile (Compound 18). LCMS m/z [M+H]: 663.2. ¹H NMR (400 MHz, CD₃OD): δ 8.27 (s, 1H), 7.28-7.22 (m, 1H), 7.02 (t, J=8.80 Hz, 1H), 5.57 (d, J=51.6 Hz, 1H), 4.80-4.60 (m, 3H), 4.40-4.30 (m, 1H), 4.15-4.00 (m, 2H), 4.00-3.80 (m, 2H), 3.45 (s, 3H), 2.90-2.80 (m, 2H), 2.75-2.50 (m, 2H), 2.48-2.05 (m, 6H).

2-Amino-7-fluoro-4-(8-fluoro-2-(((2R,7aS)-2-fluorotetrahydro-1H-pyrrolizin-7a(5H)-yl)methoxy)-4-(((1s,3R)-3-hydroxycyclobutyl)(methyl)amino)-6-(trifluoromethyl)quinazolin-7-yl)benzo[b]thiophene-3-carbonitrile (Compound 18) was separated with chiral chromatography (ChiralPak ID 2 cm×25 cm, 5 μm, Hex:EtOH=50:50 (0.5% ammonium in methanol)) to give one atropisomer, 2-amino-7-fluoro-4-((S)-8-fluoro-2-(((2R,7aS)-2-fluorotetrahydro-1H-pyrrolizin-7a(5H)-yl)methoxy)-4-(((1s,3R)-3-hydroxycyclobutyl)((methyl)amino)-6-(trifluoromethyl)quinazolin-7-yl)benzo[b]thiophene-3-carbonitrile (Compound 19) (74.9 mg, 0.113 mmol), as second peak; LCMS m/z [M+1]: 663.0. ¹H NMR (400 MHz, CD₃OD) δ 8.24 (s, 1H), 7.23-7.26 (m, 1H), 7.02-7.06 (t, 1H), 5.40 (m, 0.5H), 5.26 (m, 0.5H), 4.32-4.35 (m, 2H), 4.24-4.26 (m, 1H), 4.09-4.12 (m, 1H), 3.45 (s, 3H), 3.22-3.27 (m, 3H), 3.03-3.05 (m, 1H), 2.88-2.89 (m, 2H), 2.15-2.36 (m, 6H), 1.92-2.04 (m, 3H); and give the other atropisomer, 2-amino-7-fluoro-4-((R)-8-fluoro-2-(((2R,7aS)-2-fluorotetrahydro-1H-pyrrolizin-7a(5H)-yl)methoxy)-4-(((1s,3S)-3-hydroxycyclobutyl)((methyl)amino)-6-(trifluoromethyl)quinazolin-7-yl)benzo[bithiophene-3-carbonitrile (Compound 20) (78.2 mg, 0.118 mmol), as first peak. LCMS ESI(+) m/z 663.1 (M+1). ¹H NMR (400 MHz, CD₃OD) δ 8.24 (s, 1H), 7.26-7.23 (m, 1H), 7.06-7.02 (m, 1H), 5.40 (s, 0.5H), 5.26 (s, 0.5H), 4.37-4.32 (m, 2H), 4.26-4.24 (m, 1H), 4.12-4.09 (m, 1H), 3.45 (s, 3H), 3.27-3.22 (m, 3H), 3.05-3.03 (m, 1H), 2.91-2.85 (m, 2H), 2.36-2.15 (m, 6H), 2.04-1.92 (m, 3H).

### Synthetic Example 3: Synthesis of 2-amino-4-[4-[ethyl-(3-hydroxycyclobutyl)amino]-8-fluoro-2-[[(2R,8S)-2-fluoro-1,2,3,5,6,7-hexahydropyrrolizin-8-yl]methoxy]-6-(trifluoromethyl) quinazolin-7-yl]-7-fluoro-benzothiophene-3-carbonitrile (Compound 26)

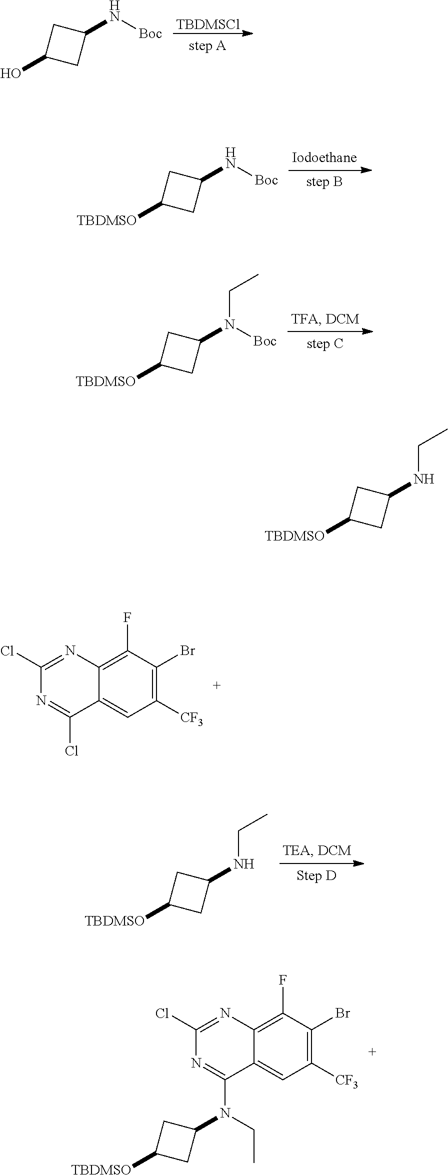

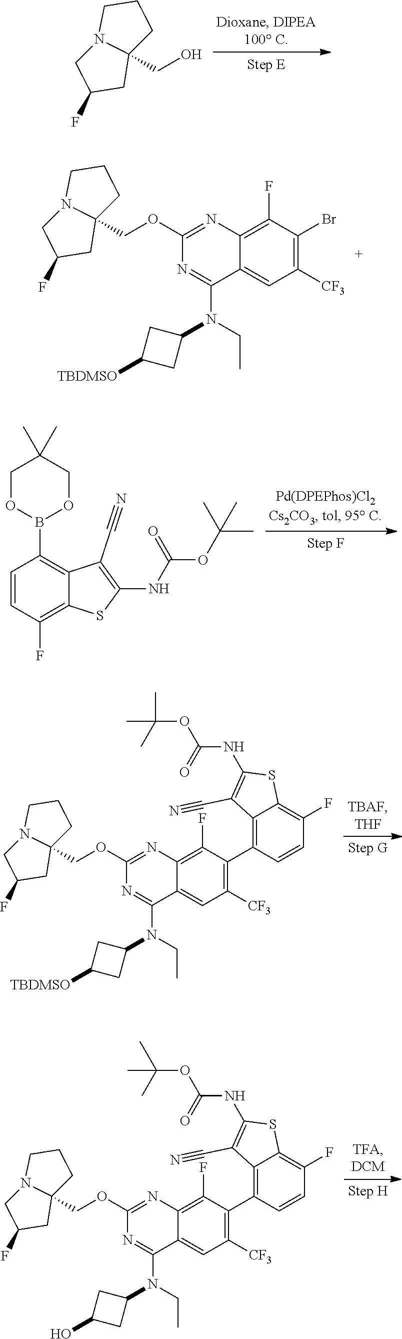

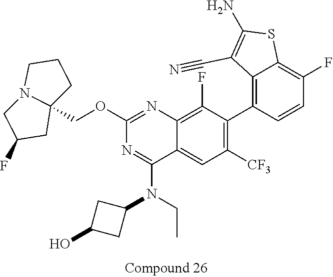

Step A: Preparation of tert-butyl N-[3-[tert-butyl(dimethyl)silyl]oxycyclobutyl]carbamate: To a solution of tert-butyldimethylchlorosilane (3.20 eq, 61.82 g, 410 mmol) in pyridine (240 mL) was added tert-butyl N-(3-hydroxycyclobutyl)carbamate (1.00 eq, 24.00 g, 128 mmol) at 25° C. After addition, the reaction mixture was stirred at 25° C. for 16 h. The mixture was quenched by water and extracted with EA (10 mL×4). The combined organic phase was washed with brine, dried over Na₂SO₄ and concentrated. The crude was purified by column chromatography on silica gel eluted with (PE/EA=100/1˜10/1) to afford tert-butyl N-[3-[tert-butyl(dimethyl)silyl]oxycyclobutyl]carbamate (40.00 g, 99.5 mmol, 77.63% yield) as a brown oil. LCMS ESI (+) m/z 302 (M+H). ¹H NMR (400 MHz, CD₃OD) δ 4.63 (s, 1H), 3.99-3.92 (m, 1H), 7.02 (t, 1H), 3.67-3.65 (m, 1H), 2.7 (d, 2H), 1.77-1.72 (m, 2H), 1.44 (s, 9H), 0.88 (s, 9H), 0.04 (s, 6H).

Step B: Preparation of tert-butyl N-[3-[tert-butyl(dimethyl)silyl]oxycyclobutyl]-N-ethyl-carbamate: To a solution of tert-butyl N-[3-[tert-butyl(dimethyl)silyl]oxycyclobutyl]carbamate (1.00 eq. 1000 mg, 3.32 mmol) in DMF (15 mL) was added NaH (2.50 eq, 332 mg, 8.29 mmol) at 0° C., and the solution was stirred at 0° C. for 30 min. Then MeI (1.30 eq, 0.35 mL, 4.31 mmol) was added at 0° C., and after addition the reaction mixture was stirred at 25° C. for 2 h. The resulting mixture was added to water and extracted with DCM (3×20 mL). The combined organic layer was washed with brine, dried over anhydrous sodium sulfate, filtered and evaporated. The crude was purified by silica gel column chromatography to give tert-butyl N-[3-[tert-butyl(dimethyl)silyl]oxycyclobutyl]-N-ethyl-carbamate (1.10 g, 2.84 mmol, 85.54% yield) as a yellow oil. LCMS ESI (+) m/z 330 (M+H).

Step C: Preparation of 3-[tert-butyl(dimethyl)silyl]oxy-N-ethyl-cyclobutanamine: To a solution of tert-butyl N-[3-[tert-butyl(dimethyl)silyl]oxycyclobutyl]-N-ethyl-carbamate (1.00 eq, 1060 mg, 3.22 mmol) in DCM (2 mL) was added 2,6-lutidine (2.00 eq, 0.75 mL, 6.43 mmol), then trimethylsilyl trifluoromethanesulfonate (1.50 eq, 0.87 mL, 4.82 mmol) in DCM (1 mL) was added dropwise at 0° C., and after addition the reaction mixture was stirred at 25° C. for 16 h. The reaction was quenched by water and extracted by DCM (30 mL×3). The combined organic phase was washed with brine, dried over sodium sulfate and concentrated. The residue was purified by column chromatography on silica gel eluted with (DCM:MEOH=10:1) to give 3-[tert-butyl(dimethyl)silyl]oxy-N-ethyl-cyclobutanamine (790 mg, 3.10 mmol, 96.35% yield) as yellow solid. LCMS ESI (+) m/z 230 (M+H). ¹H NMR (400 MHz, CD₃OD) δ 4.09-4.02 (m, 1H), 3.15-3.13 (m, 1H), 2.99-2.94 (m, 2H), 2.70-2.69 (m, 2H), 2.36-2.29 (m, 2H), 1.46-1.43 (m, 3H), 0.88 (s, 9H), 0.04 (s, 6H).

Step D: Preparation of 7-bromo-N-[3-[tert-butyl(dimethyl) silyl] oxycyclobutyl]-2-chloro-N-ethyl-8-fluoro-6-(trifluoromethyl)quinazolin-4-amine: To a solution of 7-bromo-2,4-dichloro-8-fluoro-6-(trifluoromethyl)quinazoline (1.00 eq, 200 mg, 0.550 mmol) and TEA (3.00 eq, 167 mg, 1.65 mmol) in DCM (5 mL) was added 3-[tert-butyl(dimethyl)silyl]oxy-N-ethyl-cyclobutanamine (1.00 eq, 126 mg, 0.550 mmol) in DCM (1 mL) at −30° C. under nitrogen, and after addition the reaction mixture was stirred at −30° C. for 2 h. The reaction was quenched by water and extracted by DCM (10 mL×3). The combined organic phase was washed with brine, dried over sodium sulfate and concentrated. The residue was purified by prep-TLC to give 7-bromo-N-[3-[tert-butyl(dimethyl)silyl]oxycyclobutyl]-2-chloro-N-ethyl-8-fluoro-6-(trifluoromethyl)quinazolin-4-amine (148 mg, 0.252 mmol, 45.94% yield) as a yellow solid. LCMS ESI (+) m/z 556 (M+H).

Step E: Preparation of 7-bromo-N-[3-[tert-butyl(dimethyl)silyl]oxycyclobutyl]-N-ethyl-8-fluoro-2-[[(2R,8S)-2-fluoro-1,2,3,5,6,7-hexahydropyrrolizin-8-yl]methoxy]-6-(trifluoromethyl)quinazolin-4-amine: To solution of 7-bromo-N-[3-[tert-butyl(dimethyl)silyl]oxycyclobutyl]-2-chloro-N-ethyl-8-fluoro-6-(trifluoromethyl)quinazolin-4-amine (1.00 eq, 448 mg, 0.804 mmol) in 1,4-dioxane (5 mL) was added [(2R,8S)-2-fluoro-1,2,3,5,6,7-hexahydropyrrolizin-8-yl]methanol (2.00 eq, 256 mg, 1.61 mmol) and DIEA (5.00 eq, 0.72 mL, 4.02 mmol), and after addition the reaction mixture was stirred at 110° C. for 48 h. The reaction was quenched by water and extracted by EA (10 mL×5). The combined organic phase was washed with brine, dried over sodium sulfate and concentrated. The residue was purified by column chromatography on silica gel eluted with (PE/EA=1/1) to afford 7-bromo-N-[3-[tert-butyl(dimethyl)silyl]oxycyclobutyl]-N-ethyl-8-fluoro-2-[[(2R,8S)-2-fluoro-1,2,3,5,6,7-hexahydropyrrolizin-8-yl]methoxy]-6-(trifluoromethyl)quinazolin-4-amine (260 mg, 0.363 mmol, 45.18% yield) as a yellow oil. LCMS ESI (+) m/z 679 (M+H).

Step F: Preparation of tert-butyl N-[4-[4-[[3-[tert-butyl(dimethyl)silyl]oxycyclobutyl]-ethyl-amino]-8-fluoro-2-[[(2R,8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate: To a solution of 7-bromo-N-[3-[tert-butyl(dimethyl)silyl]oxycyclobutyl]-N-ethyl-8-fluoro-2-[[(2R,8S)-2-fluoro-1,2,3,5,6,7-hexahydropyrrolizin-8-yl]methoxy]-6-(trifluoromethyl)quinazolin-4-amine (1.00 eq, 250 mg, 0.368 mmol) in 1,4-dioxane (2 mL) were added tert-butyl N-[3-cyano-4-(5,5-dimethyl-1,3,2-dioxaborinan-2-yl)-7-fluoro-benzothiophen-2-yl]carbamate (1.50 eq, 223 mg, 0.552 mmol) in 1,4-dioxane (2 mL), Cs₂CO₃ (2.50 eq, 300 mg, 0.920 mmol) and Pd(DPEPhos)Cl₂ (0.400 eq, 0.15 mL, 0.147 mmol), and after addition the reaction mixture was stirred at 95° C. for 16 h under N₂. The reaction was quenched by water and extracted with EA (30 mL×3). The combined organic phase was washed with brine, dried over sodium sulfate and concentrated. The residue was purified by prep-TLC to afford tert-butyl N-[4-[4-[[3-[tert-butyl(dimethyl)silyl]oxycyclobutyl-ethyl-amino]-8-fluoro-2-[[(2R,8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate (140 mg, 0.127 mmol, 34.60% yield) as a yellow solid. LCMS ESI (+) m/z 891 (M+H).

Step G: Preparation of tert-butyl N-[3-cyano-4-[4-[ethyl-(3-hydroxycyclobutyl)amino]-8-fluoro-2-[[(2R,8S)-2-fluoro-1,2,3,5,6,7-hexahydropyrrolizin-8-yl]methoxy]-6-(trifluoromethyl) quinazolin-7-yl]-7-fluoro-benzothiophen-2-yl]carbamate: To a solution of tert-butyl N-[4-[4-[[3-[tert-butyl(dimethyl)silyl]oxycyclobutyl]-ethyl-amino]-8-fluoro-2-[[(2R,8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate (1.00 eq, 130 mg, 0.146 mmol) in THF (3 mL) was added and 1N TBAF (3.00 eq, 0.44 mL, 0.438 mmol) at 25° C., and after addition the reaction mixture was stirred at 25° C. for 16 h. The reaction was quenched by water and extracted with EA (10 mL×3). The combined organic phase was washed with brine, dried over sodium sulfate and concentrated. The residue was purified by prep-TLC to give tert-butyl N-[3-cyano-4-[4-[ethyl-(3-hydroxycyclobutyl)amino]-8-fluoro-2-[[(2R,8S)-2-fluoro-1,2,3,5,6,7-hexahydropyrrolizin-8-yl]methoxyl-6-(trifluoromethyl)quinazolin-7-yl]-7-fluoro-benzothiophen-2-yl]carbamate (71 mg, 0.0713 mmol, 48.87% yield) as a white solid. LCMS ESI (+) m/z 777 (M+H).

Step H: Preparation of 2-amino-4-[4-[ethyl-(3-hydroxycyclobutyl)amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile: To a solution of tert-butyl N-[3-cyano-4-[4-[ethyl-(3-hydroxycyclobutyl)amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophen-2-yl]carbamate (1.00 eq, 61 mg, 0.0785 mmol) in DCM (2 mL) was added TFA (82.6 eq, 0.50 mL, 6.49 mmol) at 25° C., and after addition the reaction mixture was stirred at 25° C. for 4 h. The reaction mixture was adjusted to pH=8 with sat. NaHCO₃, and extracted with EA/THF (2/1, 10 mL×4). The combined organic phase was washed with brine, dried over sodium sulfate and concentrated. The residue was purified by prep-HPLC to afford 2-amino-4-[4-[ethyl-(3-hydroxycyclobutyl)amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (18 mg, 0.0259 mmol, 33.01% yield) as a light-yellow solid. LCMS ESI (+) m/z 677 (M+H). ¹H NMR (400 MHz, CD₃OD) S 8.24 (s, 1H), 7.22-7.20 (m, 1H), 7.02 (t, 1H), 5.54 (d, 1H), 4.74-4.58 (m, 3H), 4.41-4.29 (m, 1H), 4.07-3.85 (m, 6H), 2.86 (s, 2H), 2.41-2.27 (m, 4H), 1.70-1.62 (m, 2H), 1.38 (t, 3H), 1.02 (t, 2H).

### Synthetic Example 4: Synthesis of 7-(2-amino-3-cyano-7-fluorobenzo[b]thiophen-4-yl)-6-chloro-2-(((2R,7aS)-2-fluorotetrahydro-1H-pyrrolizin-7a(5H)-yl)methoxy)-4-(((1s,3R)-3-hydroxycyclobutyl)(methyl)amino)quinazoline-8-carbonitrile (Compound 30)

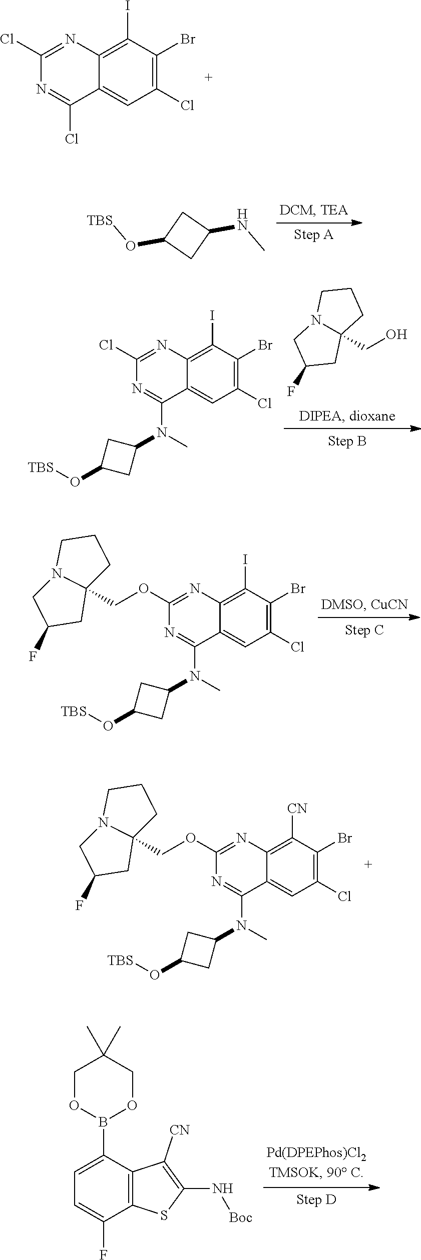

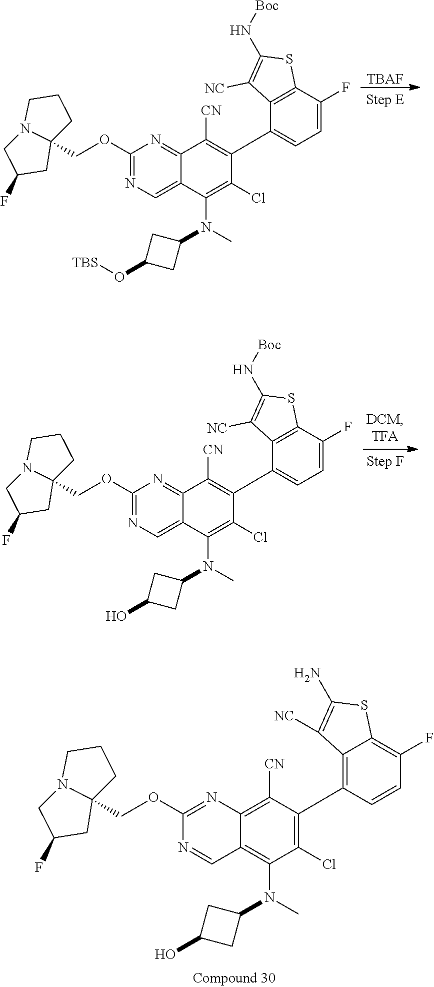

Step A: Preparation of 7-bromo-N-((1s,3s)-3-((tert-butyldimethylsilyl)oxy)cyclobutyl)-2,6-dichloro-8-iodo-N-methylquinazolin-4-amine: To a solution of 7-bromo-2,4,6-trichloro-8-iodo-quinazoline (1.00 eq, 500 mg, 1.14 mmol) in DCM (6 mL) was added TEA (3.00 eq, 346 mg, 3.42 mmol) until pH=7, then 3-[tert-butyl(dimethyl)silyl]oxy-N-methyl-cyclobutanamine (1.20 eq, 295 mg, 1.37 mmol) in DCM was added at −40° C., and after addition the reaction mixture was stirred at −40° C. for 1 h. The mixture was diluted with water and extracted with DCM (3×10 mL). The combined extracts were washed with brine, dried over sodium sulfate, filtered and concentrated. The residue was purified by prep-TLC to give 7-bromo-N-[3-[tert-butyl(dimethyl)silyl]oxycyclobutyl]-2,6-dichloro-8-iodo-N-methyl-quinazolin-4-amine (540 mg, 0.849 mmol, 74.39% yield) as a white solid. LCMS ESI (+) m/z 616 (M+H).

Step B: Preparation of 7-bromo-N-((1s,3R)-3-((tert-butyldimethylsilyl)oxy)cyclobutyl)-6-chloro-2-(((2R,7aS)-2-fluorotetrahydro-1H-pyrrolizin-7a(5H)-yl)methoxy)-8-iodo-N-methylquinazolin-4-amine: To a solution of 7-bromo-N-[3-[tert-butyl(dimethyl)silyl]oxycyclobutyl]-2,6-dichloro-8-iodo-N-methyl-quinazolin-4-amine (1.00 eq, 540 mg, 0.849 mmol) in 1,4-dioxane (6 mL) were added [(2R,8S)-2-fluoro-1,2,3,5,6,7-hexahydropyrrolizin-8-yl]methanol (2.00 eq, 268 mg, 1.68 mmol) and DIEA (5.00 eq, 0.75 mL, 4.21 mmol), and after addition the reaction mixture was stirred at 105° C. for 16 h. The mixture was diluted with water and extracted with EA (3×10 mL). The combined extracts were washed with brine, dried over sodium sulfate, filtered and concentrated. The residue was purified by prep-TLC to give 7-bromo-N-[3-[tert-butyl(dimethyl)silyl]oxycyclobutyl]-6-chloro-2-[[(2R,8S)-2-fluoro-1,2,3,5,6,7-hexahydropyrrolizin-8-yl]methoxy]-8-iodo-N-methyl-quinazolin-4-amine (233 mg, 0.302 mmol, 35.88% yield) as a yellow solid. LCMS ESI (+) m/z 739 (M+H).

Step C: Preparation of 7-bromo-4-(((1s,3R)-3-((tert-butyldimethylsilyl)oxy)cyclobutyl)methyl)amino)-6-chloro-2-(((2R,7aS)-2-fluorotetrahydro-1H-pyrrolizin-7a(5H)-yl)methoxy)quinazoline-8-carbonitrile: A solution of 7-bromo-N-[3-[tert-butyl(dimethyl)silyl]oxycyclobutyl]-6-chloro-2-[[(2R,8S)-2-fluoro-1,2,3,5,6,7-hexahydropyrrolizin-8-yl]methoxy]-8-iodo-N-methyl-quinazolin-4-amine (1.00 eq, 233 mg, 0.302 mmol) and CuCN (2.00 eq, 31 mg, 0.346 mmol) in DMSO (5 mL) was stirred at 65° C. for 16 h. The mixture was diluted with water and extracted with EA (5×10 mL). The combined extracts were washed with brine, dried over sodium sulfate, filtered, and concentrated. The residue was purified by prep-TLC to give 7-bromo-4-[[3-[tert-butyl(dimethyl)silyl]oxycyclobutyl]-methyl-amino]-6-chloro-2-[[(2R,8S)-2-fluoro-1,2,3,5,6,7-hexahydropyrrolizin-8-yl]methoxy]quinazoline-8-carbonitrile (150 mg, 0.153 mmol, 88.20% yield) as a yellow solid. LCMS ESI (+) m/z 640 (M+H).

Step D: Preparation of tert-butyl (4-(5-(((1s,3R)-3-((tert-butyldimethylsilyl)oxy)cyclobutyl)(methyl)amino)-6-chloro-8-cyano-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of 7-bromo-4-[[3-[tert-butyl(dimethyl)silyl]oxycyclobutyl]-methyl-amino]-6-chloro-2-[[(2R,8S)-2-fluoro-1,2,3,5,6,7-hexahydropyrrolizin-8-yl]methoxy]quinazoline-8-carbonitrile (1.00 eq, 150 mg, 0.220 mmol) in 1,4-dioxane (4 mL) were added tert-butyl N-[3-cyano-4-(5,5-dimethyl-1,3,2-dioxaborinan-2-yl)-7-fluoro-benzothiophen-2-yl]carbamate (1.50 eq, 133 mg, 0.329 mmol), cesium carbonate (3.00 eq, 0.36 mL, 0.657 mmol) and DPEPhosPdCl₂ (0.400 eq, 63 mg, 0.0876 mmol), and after addition the reaction mixture was stirred at 95° C. for 16 h under nitrogen. The mixture was diluted with water and extracted with EA (5×10 mL). The combined extracts were washed with brine, dried over sodium sulfate, filtered, and concentrated. The residue was purified by prep-TLC to give tert-butyl N-[4-[5-[[3-[tert-butyl(dimethyl)silyl]oxycyclobutyl]-methyl-amino]-6-chloro-8-cyano-2-[[(2R,8S)-2-fluoro-1,2,3,5,6,7-hexahydropyrrolizin-8-yl]methoxy]quinazolin-7-yl]-3-cyano-7-fluoro-benzothiophen-2-yl]carbamate (96 mg, 0.0734 mmol, 33.49% yield) as a white solid. LCMS ESI (+) m/z 850 (M+H).

Step E: Preparation of tert-butyl (4-(6-chloro-8-cyano-2-(((2R,7aS)-2-fluorotetrahydro-1H-pyrrolizin-7a(5H)-yl)methoxy)-5-(((1s,3R)-3-hydroxycyclobutyl)(methyl)amino)quinazolin-7-yl)-3-cyano-7-fluorobenzo[b]thiophen-2-yl)carbamate: To a solution of tert-butyl N-[4-[5-[[3-[tert-butyl(dimethyl)silyl]oxycyclobutyl]-methyl-amino]-6-chloro-8-cyano-2-[[(2R,8S)-2-fluoro-1,2,3,5,6,7-hexahydropyrrolizin-8-yl]methoxy]quinazolin-7-yl]-3-cyano-7-fluoro-benzothiophen-2-yl]carbamate (1.00 eq, 96 mg, 0.107 mmol) in THF (2 mL) was added 1M TBAF (2.00 eq, 0.21 mL, 0.212 mmol), and after addition the reaction mixture was stirred at 25° C. for 16 h. The mixture was diluted with water and extracted with EA (3×10 mL). The combined extracts were washed with brine, dried over sodium sulfate, filtered, and concentrated. The residue was purified by prep-TLC to give tert-butyl N-[4-[6-chloro-8-cyano-2-[[(2R,8S)-2-fluoro-1,2,3,5,6,7-hexahydropyrrolizin-8-yl]methoxy]-5-[(3-hydroxycyclobutyl)-methyl-amino]quinazolin-7-yl]-3-cyano-7-fluoro-benzothiophen-2-yl]carbamate (43 mg, 0.0491 mmol, 46.36% yield) as an orange solid. LCMS ESI (+) m/z 736 (M+H).

Step F: Preparation of 7-(2-amino-3-cyano-7-fluorobenzo[b]thiophen-4-yl)-6-chloro-2-(((2R,7aS)-2-fluorotetrahydro-1H-pyrrolizin-7a(5H)-yl)methoxy)-4-(((1s,3R)-3-hydroxycyclobutyl)methyl)amino)quinazoline-8-carbonitrile: To a solution of tert-butyl N-[4-[6-chloro-8-cyano-2-[[(2R,8S)-2-fluoro-1,2,3,5,6,7-hexahydropyrrolizin-8-yl]methoxy]-5-[(3-hydroxycyclobutyl)-methyl-amino]quinazolin-7-yl]-3-cyano-7-fluoro-benzothiophen-2-yl]carbamate (1.00 eq, 43 mg, 0.052 mmol) in DCM (3 mL) was added TFA (353 eq, 1.4 mL, 18.2 mmol) at 0° C., and after addition the reaction mixture was stirred at 0° C. for 4 h. The reaction solution was adjusted to pH 8 with saturated sodium bicarbonate aqueous solution, and extracted with EA:THF=2:1 (10 mL×4); then, the combined organic phase was washed with brine, dried over sodium sulfate and concentrated. The residue was purified by prep-HPLC to give 7-(2-amino-3-cyano-7-fluorobenzo[b]thiophen-4-yl)-6-chloro-2-(((2R,7aS)-2-fluorotetrahydro-1H-pyrrolizin-7a(5H)-yl)methoxy)-4-(((1s,3R)-3-hydroxycyclobutyl)(methyl)amino)quinazoline-8-carbonitrile (12 mg, 0.0178 mmol, 34.56% yield) as a yellow solid. LCMS ESI (+) m/z 636 (M+H). ¹H NMR (400 MHz, CD₃OD) δ 8.39 (s, 1H), 7.25-7.28 (m, III), 7.08 (t, III), 5.61 (s, 0.5H), 5.48 (s, 0.5H), 4.60-4.77 (m, 2H), 4.29-4.33 (m, 1H), 3.87-4.09 (m, 4H), 3.42-3.47 (m, 4H), 2.82-2.87 (m, 2H), 2.58-2.63 (m, 2H), 2.43-2.46 (m, 1H), 2.33-2.36 (m, 2H), 2.19-2.20 (m, 3H).

### Synthetic Example 5: Synthesis of 2-amino-7-fluoro-4-(8-fluoro-2-(((2R,7aS)-2-fluorotetrahydro-1H-pyrrolizin-7a(5H)-yl)methoxy)-4-(((1s,3R)-3-hydroxy-3-methylcyclobutyl)(methyl)amino)-6-(trifluoromethyl)quinazolin-7-yl)benzo[b]thiophene-3-carbonitrile (Compound 31)

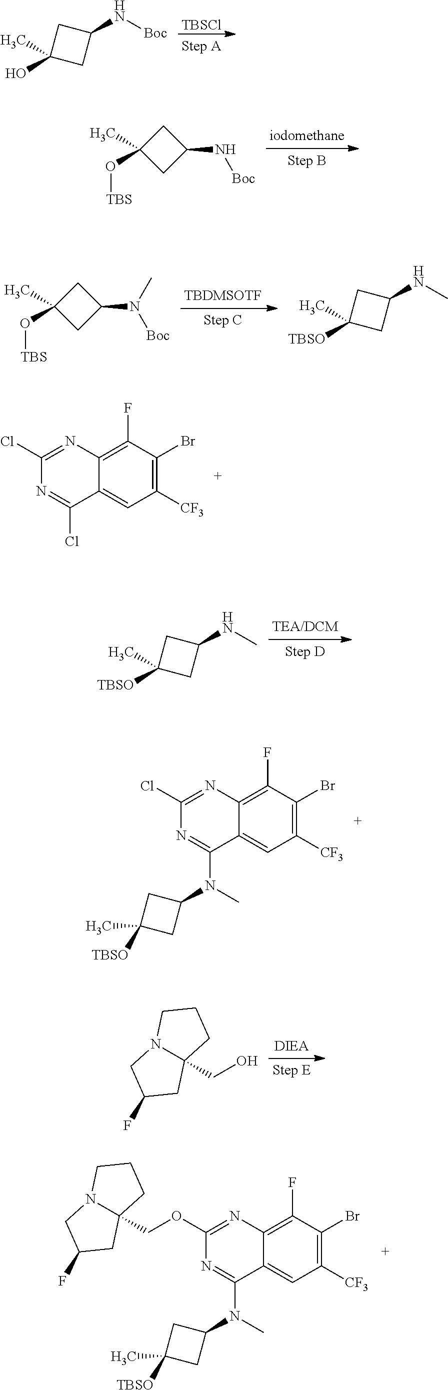

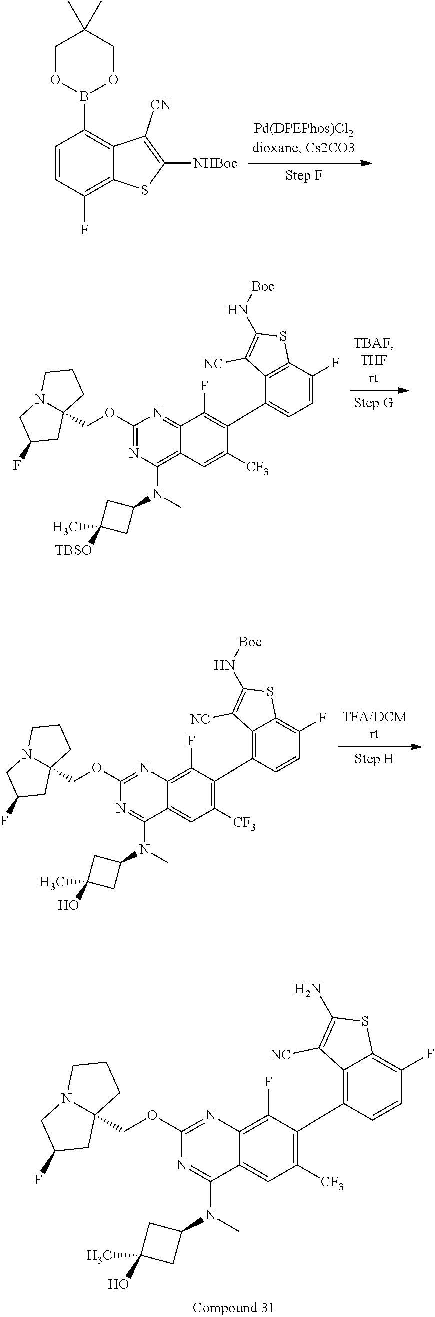 [SMILES: CC1(C)COB(c2ccc(F)c3sc(NC(=O)OC(C)(C)C)c(C#N)c23)OC1 ; CN(c1nc(OC[C@@]23CCCN2C[C@H](F)C3)nc2c(F)c(-c3ccc(F)c4sc(NC(=O)OC(C)(C)C)c(C#N)c34)c(C(F)(F)F)cc12)[C@H]1C[C@@](C)(O[Si](C)(C)C(C)(C)C)C1 ; CN(c1nc(OC[C@@]23CCCN2C[C@H](F)C3)nc2c(F)c(-c3ccc(F)c4sc(NC(=O)OC(C)(C)C)c(C#N)c34)c(C(F)(F)F)cc12)[C@H]1C[C@@](C)(O)C1 ; CN(c1nc(OC[C@@]23CCCN2C[C@H](F)C3)nc2c(F)c(-c3ccc(F)c4sc(N)c(C#N)c34)c(C(F)(F)F)cc12)[C@H]1C[C@@](C)(O)C1]

Step A: Preparation of tert-butyl ((1s,3s)-3-((tert-butyldimethylsilyl)oxy)-3-methylcyclobutyl)carbamate: To a solution of tert-butyl ((1s,3s)-3-hydroxy-3-methylcyclobutyl)carbamate (1.00 eq, 2.5 g, 12.44 mmol) in DMF (25 mL) was added tert-butyldimethylsilyl chloride (2.00 eq, 3.72 g, 24.88 mmol) and DMAP (0.10 eq, 151.9 mg, 1.244 mmol). After addition, the reaction mixture was stirred at 50° C. overnight. The reaction mixture was diluted with water (30 mL) and extracted with MTBE (30 mL×3). The combined organic layers were washed with brine (20 mL), dried over anhydrous sodium sulfate, filtered and concentrated under reduced pressure. The residue was purified by column chromatography to give tert-butyl ((1s,3s)-3-((tert-butyldimethylsilyl)oxy)-3-methylcyclobutyl)carbamate (3 g, 9.52 mmol, 76.6% yield) as a yellow oil. ¹HNMR (400 MHz, DMSO-d₆) δ 3.55-3.48 (m, 1H), 2.25-2.21 (m, 2H), 1.99-1.95 (m, 2H), 1.36 (s, 9H), 1.27 (s, 3H), 0.84 (s, 9H), 0.05 (s, 6H).

Step B: Preparation of tert-butyl ((1s,3s)-3-((tert-butyldimethylsilyl)oxy)-3-methylcyclobutyl)(methyl)carbamate: To a solution of NaH (3.00 eq, 1.14 g, 28.57 mmol) in THF (30 mL) was added tert-butyl ((1s,3s)-3-((tert-butyldimethylsilyl)oxy)-3-methylcyclobutyl)carbamate (1.00 eq, 3 g, 9.52 mmol) at 0° C. After addition, the reaction mixture was stirred at 0° C. for 30 min, then iodomethane (3.00 eq, 4.05 g, 28.57 mmol) was added dropwise at 0° C. After addition, the reaction mixture was stirred at room temperature overnight. The reaction was quenched with water (30 mL) at 0° C. The resulting mixture was extracted with EA (20 mL×3). The combined organic layers were washed with brine (20 mL), dried over anhydrous sodium sulfate, filtered, and concentrated under reduced pressure. The residue was purified by column chromatography to give tert-butyl ((1s,3s)-3-((tert-butyldimethylsilyl)oxy)-3-methylcyclobutyl)(methyl)carbamate (2.8 g, 8.51 mmol, 89.4% yield) as a yellow oil. ¹H NMR (400 MHz, CDCl₃) δ 4.15-4.13 (m, 1H), 2.81 (s, 3H), 2.27-2.25 (m, 2H), 2.21-2.19 (m, 2H), 1.47 (s, 9H), 1.37 (s, 3H), 0.90 (s, 9H), 0.09 (s, 6H).

Step C: Preparation of (1s,3s)-3-((tert-butyldimethylsilyl)oxy)-N,3-dimethylcyclobutane-1-amine: To a solution of tert-butyl ((1s,3s)-3-((tert-butyldimethylsilyl)oxy)-3-methylcyclobutyl)(methyl)carbamate (1.00 eq, 2.3 g, 6.99 mmol) in DCM (25 mL) was added trimethylsilyl trifluoromethanesulfonate (1.5 eq, 2.33 g, 10.48 mmol) and 2,6-lutidine (2 eq, 1.50 g, 13.98 mmol). After addition, the reaction mixture was stirred at 25° C. for 3 h. The reaction mixture was diluted with water (30 mL) at 0° C. The resulting mixture was extracted with DCM (30 mL×3). The combined organic layers were washed with brine (20 mL), dried over anhydrous sodium sulfate, filtered and concentrated under reduced pressure. The residue was purified by column chromatography to give (1s,3s)-3-((tert-butyldimethylsilyl)oxy)-N,3-dimethylcyclobutan-1-amine (1.5 g, 6.55 mmol, 93.7% yield) as a yellow oil. ¹H NMR (400 MHz, CDCl₃) δ 2.78-2.77 (m, 2H), 2.34-2.29 (m, 5H), 1.86-1.81 (m, 2H), 1.27 (s, 3H), 0.80 (s, 9H), 0.06 (s, 6H).

Step D: Preparation of 7-bromo-N-((1s,3s)-3-((tert-butyldimethylsilyl)oxy)-3-methylcyclobutyl)-2-chloro-8-fluoro-N-methyl-6-(trifluoromethyl)quinazolin-4-amine: To a solution of 7-bromo-2,4-dichloro-8-fluoro-6-(trifluoromethyl)quinazoline (1.00 eq, 400 mg, 1.10 mmol) in DCM (5 mL) was added TEA (3.00 eq, 334 mg, 3.30 mmol) and 3-[tert-butyl(dimethyl)silyl]oxy-N,3-dimethyl-cyclobutanamine (1.00 eq, 252 mg, 1.10 mmol) at 0° C. The mixture was stirred for 3 hours at 25° C. The mixture was quenched with water (50 mL) and extracted with DCM (50 mL×3). The combined organic layers were dried over Na₂SO₄ and concentrated to give a residue. The residue was purified by prep-TLC with EA/PE-1:10 to give 7-bromo-N-((1s,3s)-3-((tert-butyldimethylsilyl)oxy)-3-methylcyclobutyl)-2-chloro-8-fluoro-N-methyl-6-(trifluoromethyl) quinazolin-4-amine (460 mg, 0.785 mmol, 71.4% yield) as a yellow solid. LCMS EST (+) m/z 556 (M+H).

Step E: Preparation of 7-bromo-N-((1s,3R)-3-((tert-butyldimethylsilyl)oxy)-3-methylcyclobutyl)-8-fluoro-2-(((2R,7aS)-2-fluorotetrahydro-1H-pyrrolizin-7a(5H)-yl)methoxy)-N-methyl-6-(trifluoromethyl)quinazolin-4-amine: To a solution of 7-bromo-N-[3-[tert-butyl(dimethyl)silyl]oxy-3-methyl-cyclobutyl]-2-chloro-8-fluoro-N-methyl-6-(trifluoromethyl)quinazolin-4-amine (1.00 eq, 430 mg, 0.772 mmol) in DIEA (72.8 eq, 10 mL, 56.2 mmol) was added [(2R,8S)-2-fluoro-1,2,3,5,6,7-hexahydropyrrolizin-8-yl]methanol (2.00 eq, 246 mg, 1.54 mmol). The mixture was stirred for 12 hours at 100° C. The mixture was quenched with water (20 mL) and extracted with DCM (20 mL×3). The combined organic layers were dried over Na₂SO₄ and concentrated to give a residue. The residue was purified by prep-TLC with EA/PE=1:1 to give 7-bromo-N-((1s,3R)-3-((tert-butyldimethylsilyl)oxy)-3-methylcyclobutyl)-8-fluoro-2-(((2R,7aS)-2-fluorotetrahydro-1H-pyrrolizin-7a(5H)-yl)methoxy)-N-methyl-6-(trifluoromethyl)quinazolin-4-amine (300 mg, 0.433 mmol, 56.0% yield) as a yellow solid. LCMS ESI (+) m/z 679 (M+H).

Step F: Preparation of tert-butyl (4-(4-(((1s,3R)-3-((tert-butyldimethylsilyl)oxy)-3-methylcyclobutyl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-3-cyano-7-fluorobenzo[b]thiophen-2-yl)carbamate: To a solution of 7-bromo-N-[3-[tert-butyl(dimethyl)silyl]oxy-3-methyl-cyclobutyl]-8-fluoro-2-[[(2R,8S)-2-fluoro-1,2,3,5,6,7-hexahydropyrrolizin-8-yl]methoxy]-N-methyl-6-(trifluoromethyl)quinazolin-4-amine (1.00 eq, 150 mg, 0.221 mmol) in 1,4-dioxane (2 mL) were added tert-butyl N-[3-cyano-4-(5,5-dimethyl-1,3,2-dioxaborinan-2-yl)-7-fluoro-benzothiophen-2-yl]carbamate (1.50 eq, 134 mg, 0.331 mmol), Cs₂CO₃ (3.00 eq. 216 mg, 0.662 mmol), and Pd(DPEphos)Cl₂ (0.200 eq, 0.044 mL, 0.0441 mmol). The mixture was stirred for 12 hours at 100° C. under N₂. The mixture was quenched with water (10 mL) and extracted with EA (10 mL×3). The combined organic layers were dried over Na₂SO₄ and concentrated to give a residue. The residue was purified by prep-TLC with DCM/MeOH=10:1 to give tert-butyl (4-(4-(((1s,3R)-3-((tert-butyldimethylsilyl)oxy)-3-methylcyclobutyl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-3-cyano-7-fluorobenzo[b]thiophen-2-yl)carbamate (210 mg, 0.200 mmol, 90.8% yield) as a yellow solid. LCMS ESI (+) m/z 891 (M+H).

Step G: Preparation of tert-butyl (3-cyano-7-fluoro-4-(8-fluoro-2-(((2R,7aS)-2-fluorotetrahydro-1H-pyrrolizin-7a(5H)-yl)methoxy)-4-(((1s,3R)-3-hydroxy-3-methylcyclobutyl)(methyl)amino)-6-(trifluoromethyl)quinazolin-7-yl)benzo[b]thiophen-2-yl)carbamate: To a solution of tert-butyl N-[4-[4-[[3-[tert-butyl(dimethyl)silyl]oxy-3-methyl-cyclobutyl]-methyl-amino]-8-fluoro-2-[[(2R,8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate (1.00 eq, 190 mg, 0.213 mmol) in THF (5 mL) was added TBAF (5.00 eq, 279 mg, 1.07 mmol). The mixture was stirred for 2 hours at 25° C. The mixture was quenched with water (10 mL) and extracted with EA (10 mL×3). The combined organic layers were dried over Na₂SO₄ and concentrated to give a residue. The residue was purified by prep-TLC with DCM/MeOH=15:1 to give tert-butyl (3-cyano-7-fluoro-4-(8-fluoro-2-(((2R,7aS)-2-fluorotetrahydro-1H-pyrrolizin-7a(5H)-yl)methoxy)-4-(((1s,3R)-3-hydroxy-3-methylcyclobutyl)(methyl)amino)-6-(trifluoromethyl)quinazolin-7-yl)benzo[b]thiophen-2-yl)carbamate (113 mg, 0.116 mmol, 54.6% yield) as a yellow solid. LCMS ESI (+) m/z 777 (M+H).

Step H: Preparation of 2-amino-7-fluoro-4-(8-fluoro-2-(((2R,7aS)-2-fluorotetrahydro-1H-pyrrolizin-7a(5H)-yl)methoxy)-4-(((1s,3R)-3-hydroxy-3-methylcyclobutyl)(methyl)amino)-6-(trifluoromethyl)quinazolin-7-yl)benzo[b]thiophene-3-carbonitrile: To a solution of tert-butyl N-[3-cyano-7-fluoro-4-[8-fluoro-2-[[(2R,8S)-2-fluoro-1,2,3,5,6,7-hexahydropyrrolizin-8-yl]methoxy]-4-[(3-hydroxy-3-methyl-cyclobutyl)-methyl-amino]-6-(trifluoromethyl)quinazolin-7-yl]benzothiophen-2-yl]carbamate (1.00 eq, 100 mg, 0.129 mmol) in DCM (4 mL) was added TFA (403 eq, 4.0 mL, 51.9 mmol). The mixture was stirred for 0.5 h at 25° C. The mixture was adjusted to pH=8 with NaHCO₃ (aq) and extracted with DCM (10 mL×3). The combined organic layers were dried over Na₂SO₄ and concentrated to give a residue. The residue was purified by prep-HPLC to give 2-amino-7-fluoro-4-(8-fluoro-2-(((2R,7aS)-2-fluorotetrahydro-1H-pyrrolizin-7a(5H)-yl)methoxy)-4-(((1s,3R)-3-hydroxy-3-methylcyclobutyl)(methyl)amino)-6-(trifluoromethyl)quinazolin-7-yl)benzo[b]thiophene-3-carbonitrile (15.3 mg, 0.0221 mmol, 17.1% yield) as a white solid. LCMS ESI (+) m/z 677 (M+H). ¹H NMR (400 MHz, CD₃OD) δ 8.23 (s, 1H), 7.24-7.21 (m, 1H), 7.04-7.00 (m, 1H), 5.48-5.37 (m, 1H), 4.54-4.38 (m, 3H), 3.67-3.48 (m, 3H), 3.43 (s, 3H), 3.26-3.16 (m, 1H), 2.58-2.00 (m, 10H), 1.37 (s, 3H).

### Synthetic Example 6: Synthesis of 7-(2-amino-3-cyano-7-fluoro-benzothiophen-4-yl)-8-fluoro-2-[[(2R,8S)-2-fluoro-1,2,3,5,6,7-hexahydropyrrolizin-8-yl]methoxy]-4-[(3-hydroxycyclobutyl)-methyl-amino]quinazoline-6-carbonitrile (Compound 32)

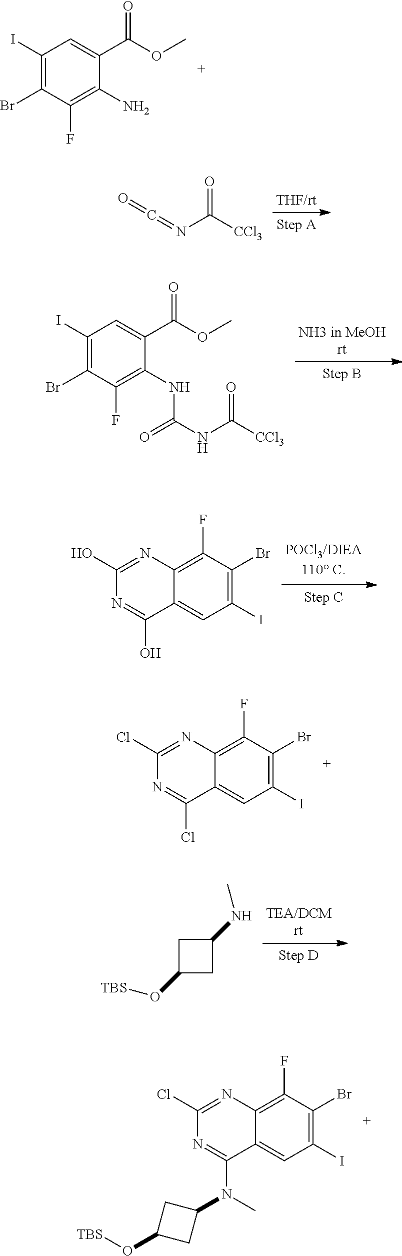

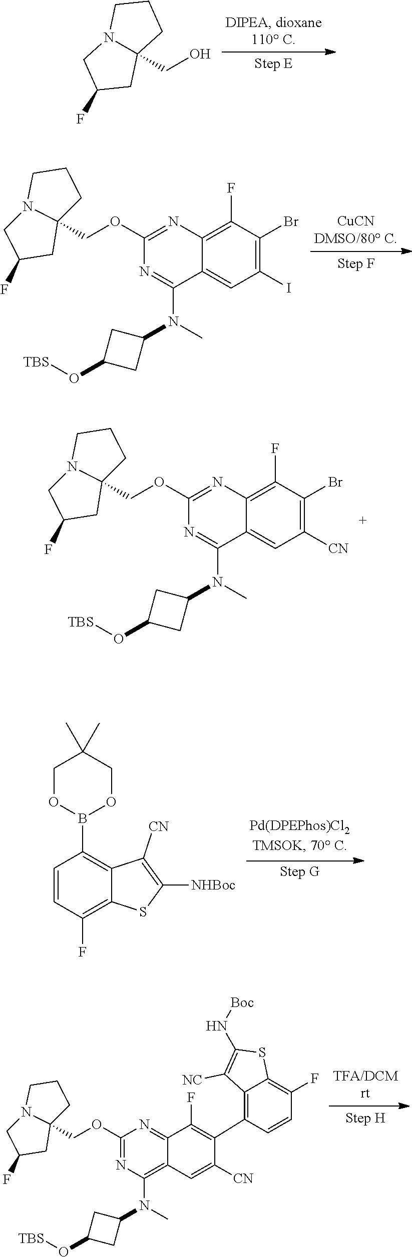

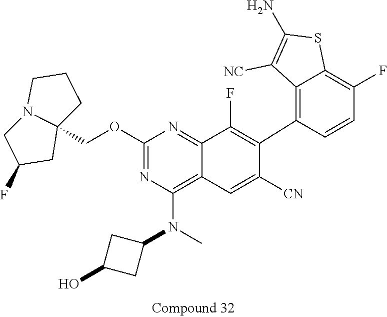

Step A: Preparation of methyl 4-bromo-3-fluoro-5-iodo-2-[(2,2,2-trichloroacetyl)carbamoylamino]benzoate: To solution of methyl 2-amino-4-bromo-3-fluoro-5-iodobenzoate (1.00 eq, 10.00 g, 26.7 mmol) in THF (100 mL) was added 2,2,2-trichloroacetyl isocyanate (1.50 eq, 7557 mg, 40.1 mmol). After addition the reaction mixture was stirred 25° C. for 1 h. The solvent was removed, and the residue was triturated with MTBE to give methyl 4-bromo-3-fluoro-5-iodo-2-[(2,2,2-trichloroacetyl)carbamoylamino]benzoate (13.00 g, 20.8 mmol, 77% yield) as a solid. LCMS ESI (+) m/z 563.7 (M+H), 585.6 (M+23). ¹HNMR (400 MHz, DMSO-d₆) δ 11.83 (brs, 1H), 10.04 (s, 1H), 8.17 (s, 1H), 3.82 (s, 3H).

Step B: Preparation of 7-bromo-8-fluoro-6-iodo-quinazoline-2,4-diol: To a solution of methyl 4-bromo-3-fluoro-5-iodo-2-[(2,2,2-trichloroacetyl)carbamoylamino]benzoate (1.00 eq, 13.00 g, 23.1 mmol) in methanol (50 mL) was added NH₃ in CH₃OH (7M) (1.00 eq, 80 mL, 23.1 mmol) at ambient temperature and stirred at ambient temperature for 1 hour. The mixture was concentrated under reduced pressure, and the residue was trituration with ether to give 7-bromo-8-fluoro-6-iodo-quinazoline-2,4-diol (9.70 g, 98% yield) as a solid. LCMS ESI (+) m/z 384.9 (M+H).

Step C: Preparation of 7-bromo-2,4-dichloro-8-fluoro-6-iodo-quinazoline: To a stirring solution of phosphorus oxychloride (23.0 eq, 27426 mg, 179 mmol) and DIPEA (5.00 eq, 5036 mg, 39.0 mmol) was added 7-bromo-8-fluoro-6-iodo-quinazoline-2,4-diol (1.00 eq, 3.00 g, 7.79 mmol) at 0° C. After addition, the resulting mixture was stirred at 110° C. for 4 hours. Once cooled down to ambient temperature, the mixture was evaporated and co-evaporated with chloroform to give 7-bromo-2,4-dichloro-8-fluoro-6-iodo-quinazoline (7.00 g, 95% yield) which used directly in next step without further purification LCMS ESI (+) m/z 421.8 (M+H).

Step D: Preparation of 7-bromo-N-[3-[tert-butyl(dimethyl)silyl]oxycyclobutyl]-2-chloro-8-fluoro-6-iodo-N-methyl-quinazolin-4-amine: To a solution of 7-bromo-2,4-dichloro-8-fluoro-6-iodo-quinazoline (1.00 eq, 700 mg, 1.66 mmol) in DCM (10 mL) was added TEA (3.00 eq, 504 mg, 4.98 mmol) and 3-[tert-butyl(dimethyl)silyl]oxy-N-methyl-cyclobutanamine (1.00 eq, 357 mg, 1.66 mmol) at −20° C. After addition, the reaction mixture was stirred at −20° C. for 1.5 h. The mixture was diluted with DCM (10 mL), washed with water and brine, dried over Na₂SO₄ and concentrated to give crude 7-bromo-N-[3-[tert-butyl(dimethyl)silyl]oxycyclobutyl]-2-chloro-8-fluoro-6-iodo-N-methyl-quinazolin-4-amine (0.95 g, 1.10 mmol, 66.7% yield) as a yellow solid. LCMS ESI (+) m/z 600.0 (M+H).

Step E: Preparation of 7-bromo-N-[3-[tert-butyl(dimethyl)silyl]oxycyclobutyl]-8-fluoro-2-[[(2R,8S)-2-fluoro-1,2,3,5,6,7-hexahydropyrrolizin-8-yl]methoxy]-6-iodo-N-methyl-quinazolin-4-amine: To a solution of 7-bromo-N-[3-[tert-butyl(dimethyl)silyl]oxycyclobutyl]-2-chloro-8-fluoro-6-iodo-N-methyl-quinazolin-4-amine (1.00 eq, 800 mg, 1.33 mmol) in 1,4-dioxane (10 mL) was added [(2R,8S)-2-fluoro-1,2,3,5,6,7-hexahydropyrrolizin-S-yl]methanol (2.00 eq, 424 mg, 2.66 mmol) and DIPEA (5.00 eq, 1.2 mL, 6.66 mmol). After addition, the reaction mixture was stirred at 110° C. for 48 hours. The solvent was removed and the residue was dissolved in EA (10 mL). The organic layer was washed with water and brine, dried over Na₂SO₄ and concentrated. The residue was purified by prep-TLC (D/M=10/1) to give 7-bromo-N-[3-[tert-butyl(dimethyl)silyl]oxycyclobutyl]-8-fluoro-2-[[(2R,8S)-2-fluoro-1,2,3,5,6,7-hexahydropyrrolizin-8-yl]methoxy]-6-iodo-N-methyl-quinazolin-4-amine (230 mg, 0.286 mmol, 21.48% yield) as a yellow solid. LCMS ESI (+) m/z 723.1 (M+H).

Step F: Preparation of 7-bromo-4-[[3-[tert-butyl(dimethyl)silyl]oxycyclobutyl]-methyl-amino]-8-fluoro-2-[[(2R,8S)-2-fluoro-1,2,3,5,6,7-hexahydropyrrolizin-8-yl]methoxy]quinazoline-6-carbonitrile: To a solution of 7-bromo-N-[3-[tert-butyl(dimethyl)silyl]oxycyclobutyl]-8-fluoro-2-[[(2R,8S)-2-fluoro-1,2,3,5,6,7-hexahydropyrrolizin-8-yl]methoxy]-6-iodo-N-methyl-quinazolin-4-amine (1.00 eq. 230 mg, 0.318 mmol) in DMSO (8 mL) was added CuCN (2.00 eq, 57 mg, 0.636 mmol). After addition, the reaction mixture was stirred at 80° C. for 3 h. The reaction mixture was poured into ice water and extracted with EA (3×10 mL). The combined organic phase was washed with water and brine, dried over Na₂SO₄ and concentrated. Then the residue was purified by prep-HPLC and then dried by lyophilization to give 7-bromo-4-[[3-[tert-butyl(dimethyl)silyl]oxycyclobutyl]-methyl-amino]-8-fluoro-2-[[(2R,8S)-2-fluoro-1,2,3,5,6,7-hexahydropyrrolizin-8-yl]methoxy]quinazoline-6-carbonitrile (35 mg, 0.0517 mmol, purity: 92%, 16.27% yield) as a yellow solid. LCMS ESI (+) m/z 622.2 (M+H).

Step G: Preparation of tert-butyl N-[4-[4-[[3-[tert-butyl(dimethyl)silyl]oxycyclobutyl]-methyl-amino]-6-cyano-8-fluoro-2-[[(2R,8S)-2-fluoro-1,2,3,5,6,7-hexahydropyrrolizin-8-yl]methoxy]quinazolin-7-yl]-3-cyano-7-fluoro-benzothiophen-2-yl]carbamate: To a solution of 7-bromo-4-[[3-[tert-butyl(dimethyl)silyl]oxycyclobutyl]-methyl-amino]-8-fluoro-2-[[(2R,8S)-2-fluoro-1,2,3,5,6,7-hexahydropyrrolizin-8-yl]methoxy]quinazoline-6-carbonitrile (1.00 eq, 35 mg, 0.0562 mmol) in 1,4-dioxane (1 mL) were added tert-butyl N-[3-cyano-4-(5,5-dimethyl-1,3,2-dioxaborinan-2-yl)-7-fluoro-benzothiophen-2-yl]carbamate (1.50 eq, 34 mg, 0.0843 mmol), TMSOK (3.00 eq. 22 mg, 0.169 mmol) and Pd(DPEphos)Cl₂ (0.500 eq, 25 mg, 0.0281 mmol). After addition, the reaction mixture was stirred at 70° C. under N₂ for 4 hours. The reaction mixture was poured into ice water and extracted with EA (3×10 mL). The combined organic phase was washed with water and brine, dried over Na₂SO₄, concentrated. The residue was purified by prep-TLC (D/M=10/1) to give tert-butyl N-[4-[4-[[3-[tert-butyl(dimethyl)silyl]oxycyclobutyl]-methyl-amino]-6-cyano-8-fluoro-2-[[(2R,8S)-2-fluoro-1,2,3,5,6,7-hexahydropyrrolizin-8-yl]methoxy]quinazolin-7-yl]-3-cyano-7-fluoro-benzothiophen-2-yl]carbamate (40 mg, 0.0408 mmol, 72.51% yield) as a white solid. LCMS ESI (+) m/z 834.1 (M+H).

Step H: Preparation of 7-(2-amino-3-cyano-7-fluoro-benzothiophen-4-yl)-8-fluoro-2-[[(2R,8S)-2-fluoro-1,2,3,5,6,7-hexahydropyrrolizin-8-yl]methoxy]-4-[(3-hydroxycyclobutyl)-methyl-amino]quinazoline-6-carbonitrile: To a solution of tert-butyl N-[3-cyano-4-[6-cyano-8-fluoro-2-[[(2R,8S)-2-fluoro-1,2,3,5,6,7-hexahydropyrrolizin-8-yl]methoxy]-4-[(3-hydroxycyclobutyl)-methyl-amino]quinazolin-7-yl]-7-fluoro-benzothiophen-2-yl]carbamate (1.00 eq, 37 mg, 0.0514 mmol) in DCM (1 mL) was added TFA (126 eq. 0.50 mL, 6.49 mmol) at −30° C. After addition, the reaction mixture was stirred at −30° C. for an hour. The mixture was neutralized with NaHCO₃ aqueous solution to pH=8 at 0° C. and extracted with DCM (3×10 mL). The organic layer was washed with water and brine, dried over Na₂SO₄ and concentrated in vacuo. Then the residue was purified by prep-HPLC and dried by lyophilization to give 7-(2-amino-3-cyano-7-fluoro-benzothiophen-4-yl)-8-fluoro-2-[[(2R,8S)-2-fluoro-1,2,3,5,6,7-hexahydropyrrolizin-8-yl]methoxy]-4-[(3-hydroxycyclobutyl)-methyl-amino]quinazoline-6-carbonitrile (8.4 mg, 0.0134 mmol, 26.10% yield) as a white solid. MS (ESI), [M+H]+=620.2. ¹H NMR (400 MHz, CD₃OD) δ 8.36 (s, 1H), 7.34-7.32 (m, 1H), 7.10-7.06 (m, 1H), 5.51 (s, 0.49H), 5.40 (s, 0.51H), 4.58-4.34 (m, 4H), 4.10-4.08 (m, 1H), 3.76-3.57 (m, 5H), 3.46 (s, 3H), 2.87 (s, 2H), 2.55-2.40 (m, 3H), 2.32-2.21 (m, 6H).

### Synthetic Example 7: 2-amino-7-fluoro-4-[8-fluoro-2-[[(2R,8S)-2-fluoro-1,2,3,5,6,7-hexahydropyrrolizin-8-yl]methoxy]-4-[[(1R,2S,3S)-3-hydroxy-2-(hydroxymethyl)cyclobutyl]-methyl-amino]-6-(trifluoromethyl)quinazolin-7-yl]benzothiophene-3-carbonitrile (Compound 37)

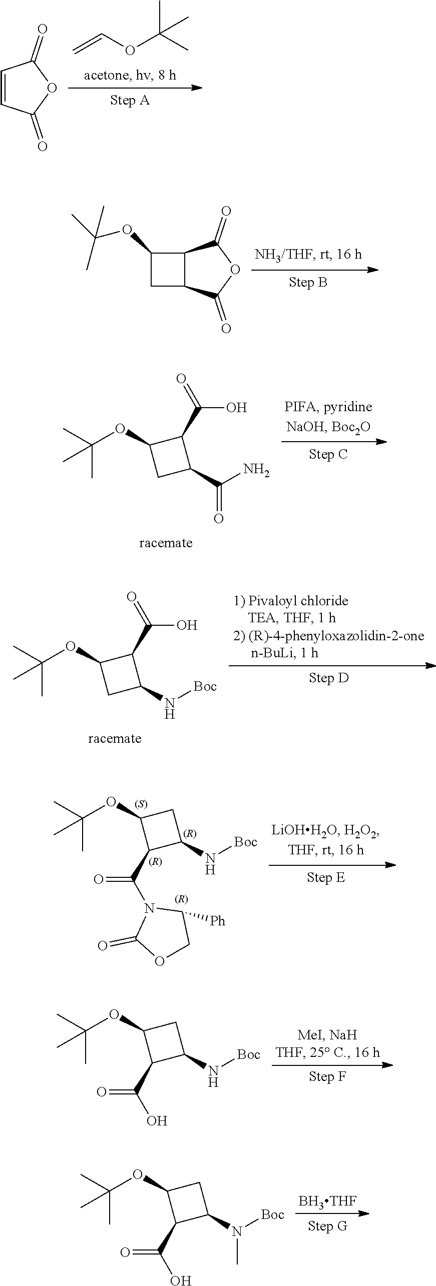

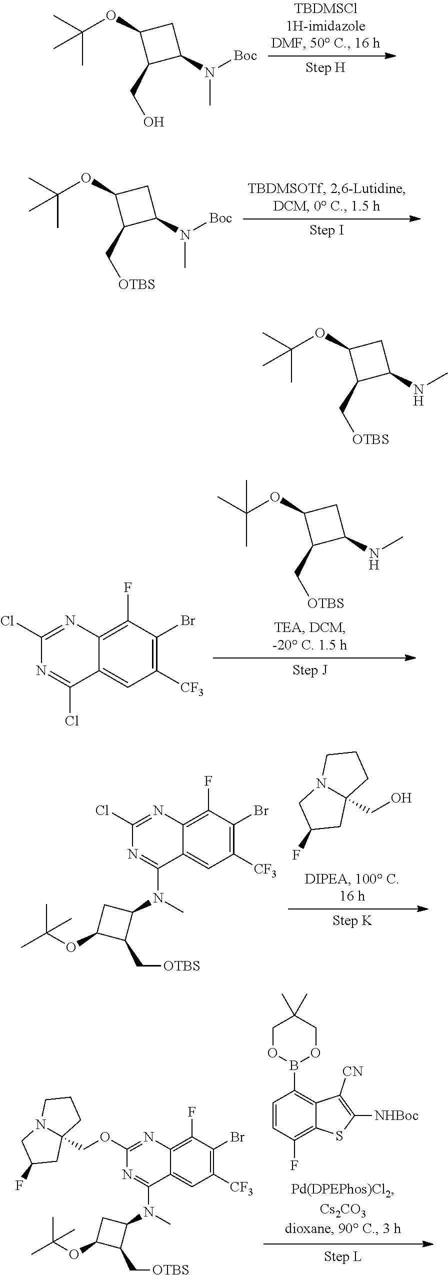

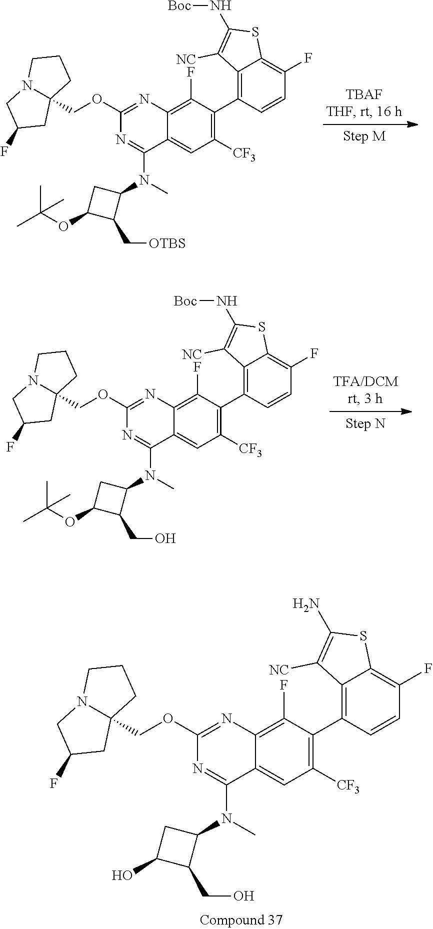 [SMILES: CN(c1nc(OC[C@@]23CCCN2C[C@H](F)C3)nc2c(F)c(-c3ccc(F)c4sc(NC(=O)OC(C)(C)C)c(C#N)c34)c(C(F)(F)F)cc12)[C@@H]1C[C@H](OC(C)(C)C)[C@@H]1CO[Si](C)(C)C(C)(C)C ; CN(c1nc(OC[C@@]23CCCN2C[C@H](F)C3)nc2c(F)c(-c3ccc(F)c4sc(NC(=O)OC(C)(C)C)c(C#N)c34)c(C(F)(F)F)cc12)[C@@H]1C[C@H](OC(C)(C)C)[C@@H]1CO ; CN(c1nc(OC[C@@]23CCCN2C[C@H](F)C3)nc2c(F)c(-c3ccc(F)c4sc(N)c(C#N)c34)c(C(F)(F)F)cc12)[C@@H]1C[C@H](O)[C@@H]1CO]

Step A: Preparation of (1S,5S,6R)-6-(tert-butoxy)-3-oxabicyclo[3.2.0]heptane-2,4-dione: A solution of furan-2,5-dione (1.00 eq, 7.20 g, 73.4 mmol) and tert-butyl vinyl ether (1.49 eq, 14 mL, 109 mmol) in acetone (2400 mL) was irradiated with a 1000 W high-pressure Hg lamp and a water-cooling jacket for 8 h. The resulting mixture was concentrated to give a crude (1S,5S,6R)-6-tert-butoxy-3-oxabicyclo[3.2.0]heptane-2,4-dione (14.00 g, 70.6 mmol, 96.19% yield) as yellow oil. ¹H NMR (400 MHz, CDCl₃) S 4.64-4.51 (m, 1H), 3.75 (t, J=7.2 Hz, 1H), 3.27-3.16 (m, 1H), 3.13-3.01 (m, 1H), 2.45-2.30 (m, 1H-), 1.21 (s, 9H).

Step B: Preparation of racemic (1S,2R,4S)-2-tert-butoxy-4-carbamoyl-cyclobutanecarboxylic acid: A solution of (1S,5S,6R)-6-tert-butoxy-3-oxabicyclo[3.2.0]heptane-2,4-dione (1.00 eq, 26.00 g, 131 mmol) in 4M NH₃/MeOH (30 mL) was stirred at 20° C. for 7 h. The resulting mixture was concentrated to give crude racemic (1S,2R,4S)-2-tert-butoxy-4-carbamoyl-cyclobutanecarboxylic acid (28.00 g, 130 mmol, 99.17% yield) as yellow oil. LCMS ESI (+) m/z 160.1 (M+H−56).

Step C: Preparation of racemic (1S,2R,4S)-2-tert-butoxy-4-(tert-butoxycarbonylamino)cyclobutanecarboxylic acid: To a solution of racemic (1S,2R,4S)-2-tert-butoxy-4-carbamoyl-cyclobutanecarboxylic acid (1.00 eq, 28.00 g, 130 mmol) in acetonitrile (300 mL) and water (300 mL) was added PIFA (1.50 eq, 83.91 g, 195 mmol). The resulting mixture was stirred at 20° C. for 1 h. Then pyridine (3.00 eq, 31 mL, 390 mmol) was added, and the mixture was stirred at 20° C. for 16 h. Acetonitrile was evaporated carefully, and the residual aqueous solution was basified with NaOH solution (2 M) to pH-9. Di-tert-butyl dicarbonate (1.30 eq, 36.87 g, 169 mmol) solution in dioxane (300 mL) was added, and the mixture was stirred overnight at room temperature. Dioxane was evaporated carefully, and the residual aqueous solution was acidified with HCl (1 M) to pH=4, followed by extraction with ethyl acetate (3×200 mL). The combined organic phases were dried over MgSO₄, filtered, and evaporated under reduced pressure. The residue was purified by column chromatography eluted with (PE/EA=2:1) to give racemic (1S,2R,4S)-2-tert-butoxy-4-(tert-butoxycarbonylamino)cyclobutanecarboxylic acid racemate (4.50 g, 15.7 mmol, 12.04%% yield) as yellow oil. LCMS ESI (−) m/z 286.4 (M−H).

Step D: Preparation of tert-butyl N-[(1R,2R,3S)-3-tert-butoxy-2-[(4R)-2-oxo-4-phenyl-oxazolidine-3-carbonyl]cyclobutyl]carbamate: To a cold (−78° C.) solution of racemic (1S.2R,4S)-2-tert-butoxy-4-(tert-butoxycarbonylamino)cyclobutanecarboxylic acid (1.00 eq, 11.70 g, 40.7 mmol) and TEA (1.20 eq, 4.94 g, 48.9 mmol) in THF (100 mL) was added pivaloyl chloride (1.05 eq, 5.3 mL, 42.8 mmol) dropwise. The mixture was stirred at 0° C. for 1 h to form the mixed anhydride and then cooled to −78° C. In a separate flask, a cold (−40° C.) solution of (R)-(−)-4-phenyl-2-oxazolidinone (1.00 eq. 6.64 g, 40.7 mmol) in THF (30 mL) was treated with a hexane solution of n-BuLi (1.00 eq, 16 mL, 40.7 mmol) and stirred for 30 min. The resulting solution was cooled to −78° C., and added by rapid cannulation to the cooled (−78° C.) solution of the mixed anhydride. After 1 h, the reaction mixture was warmed up to room temperature and treated with saturated NaHCO₃. THF was removed under reduced pressure, and the remaining aqueous solution was extracted with EA (3×200 mL). The combined organic phases were washed with brine (100 mL), dried over MgSO₄, filtered, and evaporated under reduced pressure. The residue was purified by column chromatography eluted with (PE/EA=20:1-10:1) to afford tert-butyl N-[(1R,2R,3S)-3-tert-butoxy-2-[(4R)-2-oxo-4-phenyl-oxazolidine-3-carbonyl]cyclobutyl]carbamate (4.50 g, 10.4 mmol, 25.55% yield) as yellow solid. LCMS ESI (+) m/z 277.1 (M+H−156). ¹H NMR (400 MHz, CDCl₃) δ 7.48-7.27 (m, 5H), 6.30-6.09 (m, 1H), 5.54-5.42 (m, 1H), 4.90-4.76 (m, 1H), 4.61 (t, J=8.8 Hz, 1H), 4.27-4.20 (m, 1H), 4.18-4.07 (m, 2H), 2.68-2.57 (m, 1H), 2.21-2.09 (m, 1H), 1.37 (s, 9H), 1.14 (s, 9H).

Step E: Preparation of (1R,2S,4R)-2-tert-butoxy-4-(tert-butoxycarbonylamino)cyclobutanecarboxylic acid: To an ice cold solution of tert-butyl N-[(1R,2R,3S)-3-tert-butoxy-2-[(4R)-2-oxo-4-phenyl-oxazolidine-3-carbonyl]cyclobutyl]carbamate (1.00 eq, 4.50 g, 10.4 mmol) in a 1:4 mixture of water and THF (100 mL) was added a 30% hydrogen peroxide (6.00 eq, 3.2 mL, 62.4 mmol). The resulting mixture was stirred at 0° C. for 20 min, and then a solution of LiOH·H₂O (2.00 eq, 873 mg, 20.8 mmol) in water (5 mL) was added. The mixture was stirred at room temperature overnight. Then a saturated aqueous solution of Na₂SO₃ (40 mL) and a saturated aqueous solution of NaHCO₃ (40 mL) were added successively. THF was removed under reduced pressure, and the residual aqueous solution was washed with dichloromethane. The aqueous solution was then acidified to pH=4 with an aqueous solution of HCl (0.5 M) and extracted with dichloromethane (3×100 mL). The combined organic extracts were dried over MgSO₄, filtered, and concentrated under vacuum to give (1R,2S.4R)-2-tert-butoxy-4-(tert-butoxycarbonylamino)cyclobutanecarboxylic acid (1.60 g, 5.57 mmol, 53.52% yield) as white solid. LCMS ESI (−) m/z 286.4 (M−H). ¹H NMR (400 MHz, CDCl₃) δ 5.65-5.47 (m, 1H), 4.15-3.99 (m, 2H), 3.65-3.53 (m, 1H), 2.76-2.62 (m, 1H), 2.38-2.22 (m, 1H), 1.43 (s, 9H), 1.21 (s, 9H).

Step F: Preparation of (1R,2S,4R)-2-tert-butoxy-4-[tert-butoxycarbonyl(methyl)amino]cyclobutanecarboxylic acid: To a solution of (1R,2S,4R)-2-tert-butoxy-4-(tert-butoxycarbonylamino)cyclobutanecarboxylic acid (1.00 eq. 500 mg, 1.74 mmol) in TIF (20 mL) was added 60% Nal I (10.0 eq, 417.6 mg, 10.44 mmol) at 0° C. under N₂. After addition, the mixture was stirred at 0° C. for 1 h. Then MeI (3.00 eq, 741 mg, 5.22 mmol) was added and the mixture was stirred at 20° C. for 2 h. The reaction was monitored by LCMS. The mixture was poured into water and adjusted to pH-4 with 0.5N HCl, then extracted with DCM (3 v 20 mL). The organic layer was dried over Na₂SO₄ and concentrated to give (1R,2S,4R)-2-tert-butoxy-4-[tert-butoxycarbonyl(methyl)amino]cyclobutanecarboxylic acid (450 mg, 0.551 mmol, 64.2% yield) as a yellow oil. LCMS ESI (−) m/z 300.6 (M−H). ¹H NMR (400 MHz, CDCl₃) δ 4.19-4.01 (m, 2H), 3.67-3.54 (m, 1H), 2.85 (s, 3H), 2.79-2.63 (m, 1H), 2.60-2.48 (m, 1H), 1.45 (s, 9H), 1.23 (s, 9H).

Step G: Preparation of tert-butyl N-[(1R,2S,3S)-3-tert-butoxy-2-(hydroxymethyl)cyclobutyl]-N-methyl-carbamate: To a solution of(1R,2S,4R)-2-tert-butoxy-4-[tert-butoxycarbonyl(methyl)amino]cyclobutanecarboxylic acid (1.00 eq, 480 mg, 1.59 mmol) in TIF (10 mL) was added 1N BH₃.THF (5.0 mL). The mixture was stirred at 65° C. for 2 h. The reaction was monitored by LCMS. The reaction mixture was quenched with MeOH (6 mL) and stirred at 65° C. for 1 h. The mixture was concentrated and diluted with EA. The organic layer was washed with water, dried over Na₂SO₄ and concentrated to give tert-butyl N-[(1R,2S,3S)-3-tert-butoxy-2-(hydroxymethyl)cyclobutyl]-N-methyl-carbamate (400 mg, 1.39 mmol, 87.7% yield) as a yellow oil. LCMS ESI (+) m/z 188.2 (M+H−100).

Step H: Preparation of tert-butyl N-[(1R,2S,3S)-3-tert-butoxy-2-[[tert-butyl(dimethyl)silyl]oxymethyl]cyclobutyl]-N-methyl-carbamate: To a solution of tert-butyl N-[(1R,2S,3S)-3-tert-butoxy-2-(hydroxymethyl)cyclobutyl]-N-methyl-carbamate (1.00 eq, 400 mg, 1.39 mmol) in DMF (10 mL) was added TBSCl (2.00 eq. 420 mg, 2.78 mmol) and imidazole (2.00 eq, 190 mg, 2.78 mmol). The mixture was stirred at 50° C. for 16 hours. The reaction was monitored by LCMS. The mixture was quenched with water (20 mL) and extracted with MTBE (10 mL×2). The organic layer was washed with water and brine, dried over Na₂SO₄, concentrated to give crude tert-butyl N-[(1R,2S,3S)-3-tert-butoxy-2-[[tert-butyl(dimethyl)silyl]oxymethyl]cyclobutyl]-N-methyl-carbamate (0.6 g, 1.19 mmol, 85.8% yield) as a yellow solid. ¹H NMR (400 MHz, CDCl₃) δ 3.96-3.75 (m, 4H), 2.81 (s, 3H), 2.77 (s, 1H), 2.37-2.34 (m, 2H), 1.43 (s, 9H), 1.17 (s, 9H), 0.91-0.86 (m, 9H), 0.043 (s, 6H).

Step I: Preparation of (1R,2S,3S)-3-tert-butoxy-2-[[tert-butyl(dimethyl)silyl]oxymethyl]-N-methyl-cyclobutanamine: To a solution of tert-butyl N-[(1R,2S,3S)-3-tert-butoxy-2-[[tert-butyl(dimethyl)silyl]oxymethyl]cyclobutyl]-N-methyl-carbamate (1.00 eq, 600 mg, 1.49 mmol) in DCM (20 mL) was added TBDMSOTf (1.50 eq, 504 mg, 2.24 mmol) and 2,6-lutidine (2.00 eq, 320.3 mg, 2.99 mmol). The mixture was stirred at 25° C. for 1 hour. The mixture was quenched with water (20 mL) and extracted with DCM (10 mL×2). The combined organic layers were washed with water (20 mL), brine (20 mL), dried over Na₂SO₄ and concentrated. The residue was purified by column chromatography on silica gel (DCM/MeOH=100:1) to afford (1R,2S,3S)-3-tert-butoxy-2-[[tert-butyl(dimethyl)silyl]oxymethyl]-N-methyl-cyclobutanamine (350 mg, 0.987 mmol, 66.04% yield) as yellow oil. LCMS ESI (+) m/z 302.2 (M+H).

Step J: Preparation of 7-bromo-N-[(1R,2S,3S)-3-tert-butoxy-2-[[tert-butyl(dimethyl)silyl]oxymethyl]cyclobutyl]-2-chloro-8-fluoro-N-methyl-6-(trifluoromethyl)quinazolin-4-amine: To a solution of 7-bromo-2,4-dichloro-8-fluoro-6-(trifluoromethyl)quinazoline (1.00 eq, 460 mg, 1.26 mmol) in DCM (2 mL) was added TEA (3.00 eq, 384 mg, 3.79 mmol) and (1R,2S,3S)-3-tert-butoxy-2-[[tert-butyl(dimethyl)silyl]oxymethyl-N-methyl-cyclobutanamine (1.00 eq, 381 mg, 1.26 mmol). The mixture was stirred at −30° C. for 1 hour. The reaction was monitored by LCMS. The reaction mixture was poured into water (30 mL) and extracted with DCM (3×10 mL). The combined organic layers were washed with water (20 mL) and brine (10 mL), dried over Na₂SO₄ and concentrated. The residue was purified by prep-TLC (P/E=10:1) to give 7-bromo-N-[(1R,2S,3S)-3-tert-butoxy-2-[[tert-butyl(dimethyl)silyl]oxymethyl]cyclobutyl]-2-chloro-8-fluoro-N-methyl-6-(trifluoromethyl)quinazolin-4-amine (530 mg, 0.716 mmol, 56.6% yield) as a yellow solid. ¹H NMR (400 MHz, CDCl₃) δ 8.20 (s, 1H), 4.70 (s, 1H), 4.37 (s, 1H), 4.12-3.97 (m, 2H), 3.73 (d, J=8.4 Hz, 1H), 3.48 (s, 3H), 3.17-3.16 (m, 1H), 2.68-2.56 (m, 2H), 1.19 (s, 9H), 0.68 (s, 9H), 0.01 (s, 6H).

Step K: Preparation of 7-bromo-N-[(1R,2S,3S)-3-tert-butoxy-2-[[tert-butyl(dimethyl)silyl]oxymethyl]cyclobutyl]-8-fluoro-2-[[(2R,8S)-2-fluoro-1,2,3,5,6,7 hexahydropyrrolizin-8-yl]methoxyl-N-methyl-6-(trifluoromethyl)quinazolin-4-amine: To a solution of 7-bromo-N-[(1R,2S,3S)-3-tert-butoxy-2-[[tert-butyl(dimethyl)silyl]oxymethyl]cyclobutyl]-2-chloro-8-fluoro-N-methyl-6-(trifluoromethyl)quinazolin-4-amine (1.00 eq, 500 mg, 0.795 mmol) in DIEA (42.4 eq, 6.0 mL, 33.7 mmol) was added [(2R,8S)-2-fluoro-1,2,3,5,6,7-hexahydropyrrolizin-8-yl]methanol (2.50 eq, 316 mg, 1.99 mmol). The mixture was stirred at 100° C. for 16 hours. The reaction was monitored by LCMS. The reaction mixture was poured into water (10 mL) and extracted with EA (3×5 mL). The combined organic layers were washed with water (10 mL) and brine (10 mL), dried over Na₂SO₄ and concentrated. The residue was purified by prep-TLC (D/M=10:1) to give 7-bromo-N-[(1R,2S,3S)-3-tert-butoxy-2-[[tert-butyl(dimethyl)silyl]oxymethyl]cyclobutyl]-8-fluoro-2-[[(2R,8S)-2-fluoro-1,2,3,5,6,7-hexahydropyrrolizin-8-yl]methoxy]-N-methyl-6-(trifluoromethyl)quinazolin-4-amine (320 mg, 0.362 mmol, 45.1% yield) as a yellow solid. LCMS ESI (+) m/z 751.2 (M+H).

Step L: Preparation of tert-butyl N-[4-[4-[[(1R,2S,3S)-3-tert-butoxy-2-[[tert-butyl(dimethyl)silyl]oxymethyl]cyclobutyl]-methyl-amino]-8-fluoro-2-[[(2R,8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate: To a solution of 7-bromo-N-[(1R,2S,3S)-3-tert-butoxy-2-[[tert-butyl(dimethyl)silyl]oxymethyl]cyclobutyl]-8-fluoro-2-[[(2R,8S)-2-fluoro-1,2,3,5,6,7-hexahydropyrrolizin-8-yl]methoxy]-N-methyl-6-(trifluoromethyl)quinazolin-4-amine (1.00 eq, 230 mg, 0.306 mmol) in 1,4-dioxane (8 mL) were added tert-butyl N-[3-cyano-4-(5,5-dimethyl-1,3,2-dioxaborinan-2-yl)-7-fluoro-benzothiophen-2-yl]carbamate (1.50 eq, 186 mg, 0.459 mmol), Cs₂CO₃ (3.00 eq, 299 mg, 0.918 mmol), and Pd(DPEphos)Cl₂ (0.500 eq, 109.8 mg, 0.153 mmol). The mixture was stirred at 90° C. under N₂ for 3 hours. The reaction was monitored by LCMS. The reaction mixture was poured into water (10 mL) and extracted with EA (3×5 mL). The combined organic layers were washed with water (10 mL) and brine (10 mL), dried over Na₂SO₄ and concentrated. The residue was purified by prep-TLC (D/M=10:1) to give crude tert-butyl N-[4-[4-[[(1R,2S,3S)-3-tert-butoxy-2-[[tert-butyl(dimethyl)silyl]oxymethyl]cyclobutyl]-methyl-amino]-8-fluoro-2-[[(2R,8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate (230 mg, 0.191 mmol, 62.4% yield) as a yellow solid. LCMS EST (+) m/z 963.2 (M+H).

Step M: Preparation of tert-butyl N-[4-[4-[[(1R,2S,3S)-3-tert-butoxy-2-(hydroxymethyl)cyclobutyl]-methyl-amino]-8-fluoro-2-[[(2R,8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate: To a solution of tert-butyl N-[4-[4-[[(1R,2S,3S)-3-tert-butoxy-2-[[tert-butyl(dimethyl)silyl]oxymethyl]cyclobutyl]-methyl-amino]-8-fluoro-2-[[(2R,8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate (1.00 eq, 230 mg, 0.239 mmol) in THF (10 mL) was added 1N TBAF (10.0 eq, 2.3 mL, 2.39 mmol). The mixture was stirred at 25° C. for 16 hours. The reaction was monitored by LCMS. The reaction mixture was poured into water (30 mL) and extracted with EA (3×20 mL). The combined organic layers were washed with water (30 mL) and brine (30 mL), dried over Na₂SO₄ and concentrated. Then the residue was purified by prep-HPLC and dried by lyophilization to afford tert-butyl N-[4-[4-[[(1R,2S,3S)-3-tert-butoxy-2-(hydroxymethyl)cyclobutyl]-methyl-amino]-8-fluoro-2-[[(2R,8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate (50 mg, 0.0588 mmol, 23.7% yield) as a yellow solid. LCMS ESI (+) m/z 849.3 (M+H)

Step N: Preparation of 2-amino-7-fluoro-4-[8-fluoro-2-[[(2R,8S)-2-fluoro-1,2,3,5,6,7-hexahydropyrrolizin-8-yl]methoxy]-4-[[(1R,2S,3S)-3-hydroxy-2-(hydroxymethyl)cyclobutyl]-methyl-amino]-6-(trifluoromethyl)quinazolin-7-yl]benzothiophene-3-carbonitrile: To a solution of tert-butyl N-[4-[4-[[(1R,2S,3S)-3-tert-butoxy-2-(hydroxymethyl)cyclobutyl]-methyl-amino]-8-fluoro-2-[[(2R,8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate (1.00 eq. 15 mg, 0.0177 mmol) in DCM (1 mL) was added TFA (735 eq, 1.0 mL, 13.0 mmol). The mixture was stirred at 25° C. for 2 hours. The reaction was monitored by LCMS. The mixture was neutralized with NaHCO₃ aqueous solution to pH=8 at 0° C., and extracted with DCM (3×5 mL). The organic layer was washed with water and brine, dried over Na₂SO₄ and concentrated in vacuo. Then the residue was purified by prep-HPLC and dried by lyophilization to afford the 2-amino-7-fluoro-4-[8-fluoro-2-[[(2R,8S)-2-fluoro-1,2,3,5,6,7-hexahydropyrrolizin-8-yl]methoxy]-4-[[(1R,2S,3S)-3-hydroxy-2-(hydroxymethyl)cyclobutyl]-methyl-amino]-6-(trifluoromethyl)quinazolin-7-yl]benzothiophene-3-carbonitrile (3.4 mg, 0.00485 mmol, 27.47% yield) as a white solid. LCMS ESI (+) m/z 693.3 (M+H). ¹H NMR (400 MHz, CD₃OD) δ 8.36 (s, 1H), 7.23-7.20 (m, 1H), 7.04-6.99 (m, 1H), 5.52 (s, 0.5H), 5.39 (s, 0.5H), 4.53-4.33 (m, 4H), 3.98-3.88 (m, 1H), 3.75-3.35 (m, 4H), 3.52 (s, 3H), 3.28-3.17 (m, 2H), 2.85-1.93 (m, 10H).

### Synthetic Example 8: Synthesis of 2-amino-7-fluoro-4-(8-fluoro-2-(((2R,7aS)-2-fluorotetrahydro-1H-pyrrolizin-7a(5H)-yl)methoxy)-4-(4-(hydroxymethyl)piperidin-1-yl)-6-(trifluoromethyl)quinazolin-7-yl)benzo[b]thiophene-3-carbonitrile (Compound 44)

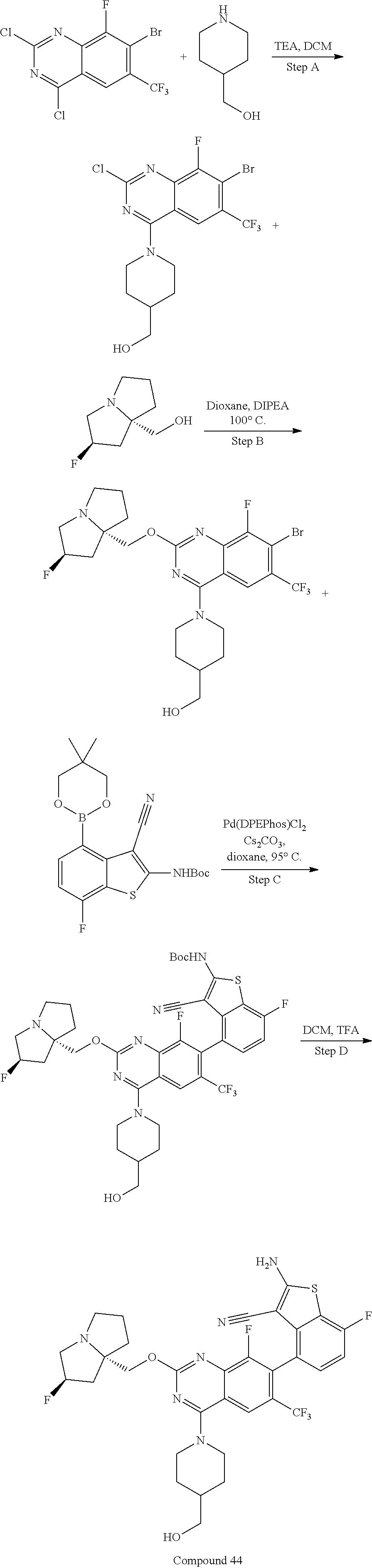

Step A: Preparation of (1-(7-bromo-2-chloro-8-fluoro-6-(trifluoromethyl)quinazolin-4-yl)piperidin-4-yl)methanol: To a solution of 7-bromo-2,4-dichloro-8-fluoro-6-(trifluoromethyl)quinazoline (1.00 eq, 500 mg, 1.37 mmol) and TEA (3.00 eq, 417 mg, 4.12 mmol) in DCM (8 mL) was added 4-piperidylmethanol (1.00 eq, 158 mg, 1.37 mmol) at −40° C., and after addition the reaction mixture was stirred at −40° C. for 1 h, then stirred at 25° C. for 1 h. The resulting mixture was extracted with DCM (25 mL×3). The combined organic layers were washed with brine (15 mL), dried over anhydrous sodium sulfate, filtered and concentrated under reduced pressure to dryness. The crude product was purified by silica gel column chromatography (PE/EA=3/1) to give [1-[7-bromo-2-chloro-8-fluoro-6-(trifluoromethyl)quinazolin-4-yl]-4-piperidyl]methanol (417 mg, 0.750 mmol, 54.55% yield) as a yellow solid. LCMS ESI (+) m/z 442.2 (M+H).

Step B: Preparation of (1-(7-bromo-8-fluoro-2-(((2R,7aS)-2-fluortetrahydro-1H-pyrrolizin-7a(5H)-yl)methoxy)-6-(trifluoromethyl)quinazolin-4-yl)piperidin-4-yl)methanol: To a solution of [1-[7-bromo-2-chloro-8-fluoro-6-(trifluoromethyl)quinazolin-4-yl]-4-piperidyl]methanol (1.00 eq, 367 mg, 0.829 mmol) in 1,4-dioxane (2 mL) was added [(2R,8S)-2-fluoro-1,2,3,5,6,7-hexahydropyrrolizin-8-yl]methanol (2.00 eq, 264 mg, 1.66 mmol) and DIPEA (5.00 eq, 0.74 mL, 4.15 mmol), and after addition the reaction mixture was stirred at 100° C. for 24 hours. The mixture was then diluted with water and extracted with EA (3×20 mL). The combined extracts were washed with brine, dried over sodium sulfate, filtered, and concentrated. The residue was purified by prep-TLC to give (1-(7-bromo-8-fluoro-2-(((2R,7aS)-2-fluorotetrahydro-1H-pyrrolizin-7a(5H)-yl)methoxy)-6-(trifluoromethyl)quinazolin-4-yl)piperidin-4-yl)methanol (300 mg, 0.531 mmol, 64.05% yield) as a yellow solid. LCMS ESI (+) m/z 565.2 (M+H).

Step C: Preparation of tert-butyl (3-cyano-7-fluoro-4-(8-fluoro-2-(((2R,7aS)-2-fluorotetrahydro-1H-pyrrolizin-7a(5H)-yl)methoxy)-4-(4-(hydroxymethyl)piperidin-1-yl)-6-(trifluoromethyl)quinazolin-7-yl)benzo[b]thiophen-2-yl)carbamate: To a solution of tert-butyl N-[3-cyano-4-(5,5-dimethyl-1,3,2-dioxaborinan-2-yl)-7-fluoro-benzothiophen-2-yl]carbamate (1.50 eq, 322 mg, 0.796 mmol) in 1,4-dioxane (9 mL) were added [1-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-4-piperidyl]methanol (1.00 eq, 300 mg, 0.531 mmol), Cs₂CO₃ (3.00 eq, 1.59 mmol), and DPEPhosPdCl₂ (0.400 eq, 152 mg, 0.212 mmol), and after addition the reaction mixture was stirred at 95° C. for 16 h under N₂. The solvent was removed, and the residue was dissolved in EA (25 mL), washed with water (5 mL) and brine (5 mL), dried over sodium sulfate, filtered and concentrated. The residue was purified by prep-HPLC to give tert-butyl N-[3-cyano-7-fluoro-4-[8-fluoro-2-[[(2R,8S)-2-fluoro-1,2,3,5,6,7-hexahydropyrrolizin-8-yl]methoxy]-4-[4-(hydroxymethyl)-1-piperidyl]-6-(trifluoromethyl)quinazolin-7-yl]benzothiophen-2-yl]carbamate (60 mg, 0.0734 mmol, 13.83% yield) as a yellow solid. LCMS ESI (+) m/z 777.3 (M+H).

Step D: Preparation of 2-amino-7-fluoro-4-(8-fluoro-2-(((2R,7aS)-2-fluorotetrahydro-1H-pyrrolizin-7a(5H)-yl)methoxy)-4-(4-(hydroxymethyl)piperidin-1-yl)-6-(trifluoromethyl)quinazolin-7-yl)benzo[b]thiophene-3-carbonitrile: To a solution of tert-butyl N-[3-cyano-7-fluoro-4-[8-fluoro-2-[[(2R,8S)-2-fluoro-1,2,3,5,6,7-hexahydropyrrolizin-8-yl]methoxy]-4-[4-(hydroxymethyl)-1-piperidyl]-6-(trifluoromethyl)quinazolin-7-yl]benzothiophen-2-yl]carbamate (1.00 eq, 60 mg, 0.0772 mmol) in DCM (3 mL) was added TFA (168 eq. 1.0 mL, 13.0 mmol) at 0° C., and after addition the reaction mixture was stirred at 0° C. for 4 h. The solvent was removed and the residue was purified by prep-HPLC to give 2-amino-7-fluoro-4-[8-fluoro-2-[[(2R,8S)-2-fluoro-1,2,3,5,6,7-hexahydropyrrolizin-8-yl]methoxy]-4-[4-(hydroxymethyl)-1-piperidyl]-6-(trifluoromethyl)quinazolin-7-yl]benzothiophene-3-carbonitrile (27 mg, 0.0392 mmol, 50.78% yield) as a yellow solid. LCMS ESI (+) m/z 677.1 (M+H). ¹H NMR (400 MHz, CD₃OD) δ 8.16 (s, 1H), 7.21-7.24 (m, 1H), 7.02 (t, 1H), 5.61 (s, 0.5H), 5.48 (s, 0.5H), 4.57-4.74 (m, 4H), 3.81-4.03 (m, 3H), 3.38-3.51 (m, 5H), 3.52-3.74 (m, 2H), 2.33-2.41 (m, 3H), 2.14 (s, 1H), 1.96-1.99 (m, 3H), 1.47-1.53 (m, 2H).

### Synthetic Example 9: Synthesis of 2-amino-7-fluoro-4-(8-fluoro-2-(((2R,7aS)-2-fluorotetrahydro-1H-pyrrolizin-7a(5H)-yl)methoxy)-4-((2-hydroxyethyl)(methyl)amino)-6-(trifluoromethyl)quinazolin-7-yl)benzo[b]thiophene-3-carbonitrile (Compound 70)

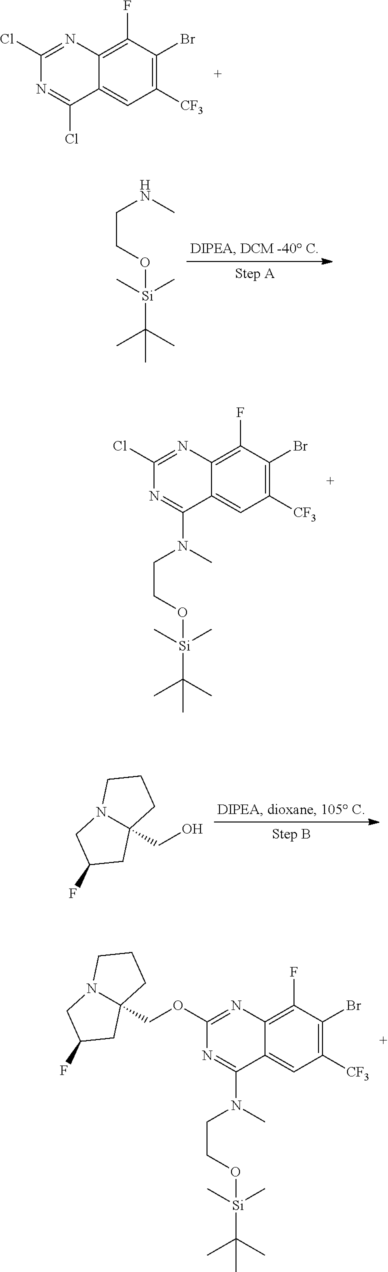

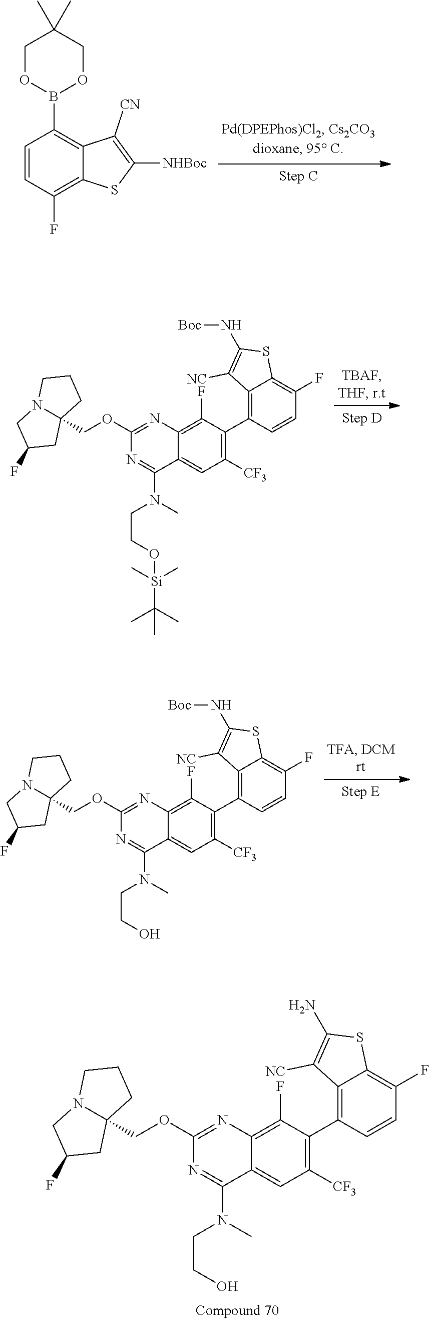 [SMILES: CC1(C)COB(c2ccc(F)c3sc(NC(=O)OC(C)(C)C)c(C#N)c23)OC1 ; CN(CCO[Si](C)(C)C(C)(C)C)c1nc(OC[C@@]23CCCN2C[C@H](F)C3)nc2c(F)c(-c3ccc(F)c4sc(NC(=O)OC(C)(C)C)c(C#N)c34)c(C(F)(F)F)cc12 ; CN(CCO)c1nc(OC[C@@]23CCCN2C[C@H](F)C3)nc2c(F)c(-c3ccc(F)c4sc(NC(=O)OC(C)(C)C)c(C#N)c34)c(C(F)(F)F)cc12 ; CN(CCO)c1nc(OC[C@@]23CCCN2C[C@H](F)C3)nc2c(F)c(-c3ccc(F)c4sc(N)c(C#N)c34)c(C(F)(F)F)cc12]

Step A: Preparation of 7-bromo-N-(2-((tert-butyldimethylsilyl)oxy)ethyl)-2-chloro-8-fluoro-N-methyl-6-(trifluoromethyl)quinazolin-4-amine: A solution of 7-bromo-2,4-dichloro-8-fluoro-6-(trifluoromethyl)quinazoline (1.00 eq, 200 mg, 0.550 mmol)₂-[tert-butyl(dimethyl)silyl]oxy-N-methyl-ethanamine (1.00 eq. 104 mg, 0.550 mmol) and DIEA (3.00 eq. 0.29 mL, 1.65 mmol) in DCM (5 mL) was stirred at 25° C. for 16 h. The mixture was then diluted with water and extracted with EA(3×200 mL). The combined extracts were washed with brine, dried over sodium sulfate, filtered and concentrated. The residue was purified by prep-TLC to give 7-bromo-N-[2-[tert-butyl(dimethyl)silyl]oxyethyl]-2-chloro-8-fluoro-N-methyl-6-(trifluoromethyl)quinazolin-4-amine (250 mg, 0.460 mmol, 83.62% yield) as a yellow solid. LCMS ESI (+) m/z 515.9 (M+H).

Step B: Preparation of 7-bromo-N-(2-((ter-butyldimethylsilyl)oxy)ethyl)-8-fluoro-2-(((2R,7aS)-2-fluorotetrahydro-1H-pyrrolizin-7(5H)yl)methoxy-N-methyl-6-(trifluoromethyl)quinazolin-4-amine: To a solution of 7-bromo-N-[2-[tert-butyl(dimethyl)silyl]oxyethyl]-2-chloro-8-fluoro-N-methyl-6-(trifluoromethyl)quinazolin-4-amine (1.00 eq, 240 mg, 0.464 mmol) in 1,4-dioxane (5 mL) was added [(2R,8S)-2-fluoro-1,2,3,5,6,7-hexahydropyrrolizin-8-yl]methanol (3.00 eq, 222 mg, 1.39 mmol) and DIPEA (5.00 eq, 0.41 mL, 2.32 mmol), and after addition the reaction mixture was stirred at 105° C. for 16 h. The mixture was then diluted with water and extracted with EA (3×20 mL). The combined extracts were washed with brine, dried over sodium sulfate, filtered and concentrated, the residue was purified by prep-TLC to give 7-bromo-N-[2-[tert-butyl(dimethyl)silyl]oxyethyl]-8-fluoro-2-[[(2R,8S)-2-fluoro-1,2,3,5,6,7-hexahydropyrrolizin-8-yl]methoxy]-N-methyl-6-(trifluoromethyl)quinazolin-4-amine (110 mg, 0.158 mmol, 34.07% yield) as a yellow solid. LCMS EST (+) m/z 639.0 (M+H).

Step C: Preparation of tert-butyl (4-(4-((2-((tert-butyldimethylsilyl)oxy)ethyl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-3-cyano-7-fluorobenzo[b]thiophen-2-yl)carbamate: To a solution of tert-butyl N-[3-cyano-4-(5,5-dimethyl-1,3,2-dioxaborinan-2-yl)-7-fluoro-benzothiophen-2-yl]carbamate (2.00 eq, 126 mg, 0.313 mmol) in 1,4-dioxane (3 mL) were added 7-bromo-N-[2-[tert-butyl(dimethyl)silyl]oxyethyl]-8-fluoro-2-[[(2R,8S)-2-fluoro-1,2,3,5,6,7-hexahydropyrrolizin-8-yl]methoxy]-N-methyl-6-(trifluoromethyl)quinazolin-4-amine (1.00 eq, 100 mg, 0.156 mmol), Cs₂CO₃ (2.50 eq. 127 mg, 0.391 mmol) and Pd(DPEPhos)Cl₂ (0.400 eq, 0.062 mL, 0.0625 mmol), and after addition the reaction mixture was stirred at 95° C. for 16 h under N₂. The mixture was then diluted with water and extracted with EA (3×20 mL). The combined extracts were washed with brine, dried over sodium sulfate, filtered and concentrated, the residue was purified by prep-TLC to give tert-butyl N-[4-[4-[2-[tert-butyl(dimethyl)silyl]oxyethyl-methyl-amino]-8-fluoro-2-[[(2R,8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate (70 mg, 0.0592 mmol, 37.88% yield) as a yellow solid. LCMS ESI (+) m/z 851.3 (M+H).

Step D: Preparation of tert-butyl (3-cyano-7-fluoro-4-(8-fluoro-2-(((2R,7aS)-2-fluorotetrahydro-1H-pyrrolizin-7a(5H)-yl)methoxy)-4-((2-hydroxyethyl)(methyl)amino)-6-(trifluoromethyl)quinazolin-7-yl)benzo[b]thiophen-2-yl)carbamate: To a solution of tert-butyl N-[4-[4-[2-[tert-butyl(dimethyl)silyl]oxyethyl-methyl-amino]-8-fluoro-2-[l(2R.8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate (1.00 eq, 60 mg, 0.0705 mmol) in THF (2 mL) was added 1N TBAF (2.00 eq, 0.14 mL, 0.141 mmol), and after addition the reaction mixture was stirred at 25° C. for 16 h. The mixture was then diluted with water and extracted with EA (3×20 mL). The combined extracts were washed with brine, dried over sodium sulfate, filtered and concentrated, the residue was purified by prep-TLC to give tert-butyl N-[3-cyano-7-fluoro-4-[8-fluoro-2-[[(2R,8S)-2-fluoro-1,2,3,5,6,7-hexahydropyrrolizin-8-yl]methoxy]-4-[2-hydroxyethyl(methyl)amino]-6-(trifluoromethyl)quinazolin-7-yl]benzothiophen-2-yl]carbamate (25 mg, 0.0305 mmol, 43.32% yield) as a yellow solid. LCMS ESI (+) m/z 737.1 (M+H).

Step E: Preparation of 2-amino-7-fluoro-4-(8-fluoro-2-(((2R,7aS)-2-fluorotetrahydro-1H-pyrrolizin-7a(5H)-yl)methoxy)-4-((2-hydroxyethyl)(methyl)amino)-6-(trifluoromethyl)quinazolin-7-yl)benzo[b]thiophene-3-carbonitrile: A solution of tert-butyl N-[3-cyano-7-fluoro-4-[8-fluoro-2-[[(2R,8S)-2-fluoro-1,2,3,5,6,7-hexahydropyrrolizin-8-yl]methoxy]-4-[2-hydroxyethyl(methyl)amino]-6-(trifluoromethyl)quinazolin-7-yl]benzothiophen-2-yl]carbamate (1.00 eq, 28 mg, 0.0380 mmol) and TFA (171 eq, 0.50 mL, 6.49 mmol) in DCM (2 mL) was stirred at 25° C. for 3 h. The solvent was removed under vacuum, the residue was purified by prep-HPLC to give 2-amino-7-fluoro-4-[8-fluoro-2-[[(2R,8S)-2-fluoro-1,2,3,5,6,7-hexahydropyrrolizin-8-yl]methoxy]-4-[2-hydroxyethyl(methyl)amino]-6-(trifluoromethyl)quinazolin-7-yl]benzothiophene-3-carbonitrile (7.5 mg, 0.0107 mmol, 28.14% yield) as light yellow solid. LCMS EST (+) m/z 637.0 (M+H). ¹H NMR (400 MHz, CD₃OD) δ 8.65 (s, 1H), 7.21 (t, 1H), 7.02 (t, 1H), 5.61 (s, 0.5H), 5.48 (s, 0.5H), 4.74-4.57 (m, 2H), 4.07-3.86 (m, 6H), 3.70-3.55 (m, 3H), 3.49-3.41 (m, 1H), 2.71-2.13 (m, 7H).

### Synthetic Example 10: Synthesis of 2-amino-4-[4-[ethyl(methyl)amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (Compound 81), 2-amino-4-[4-[ethyl(methyl)aminol-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (Compound 1%), and 2-amino-4-[4-[ethyl(methyl)amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (Compound 197)

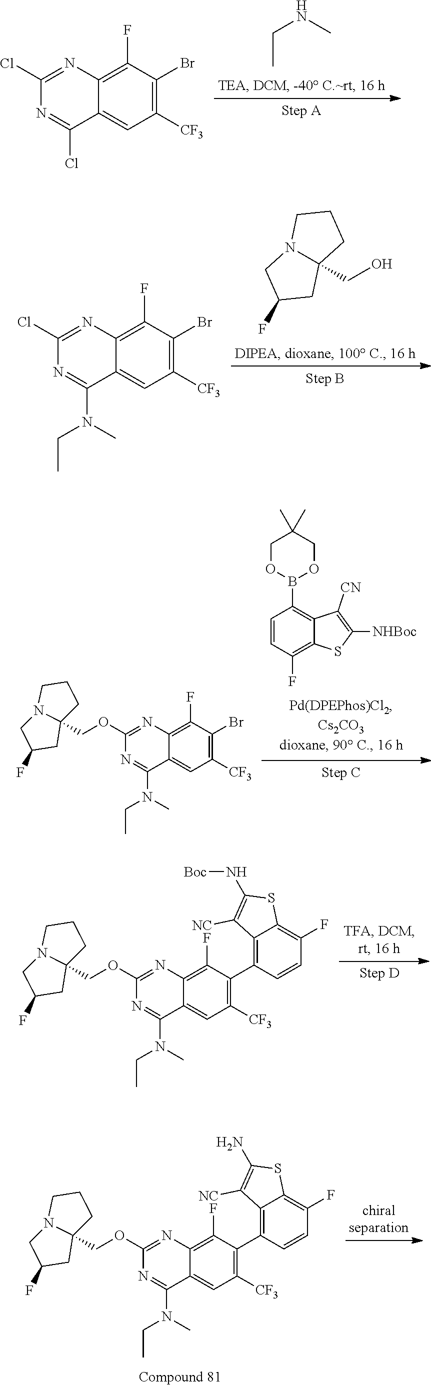

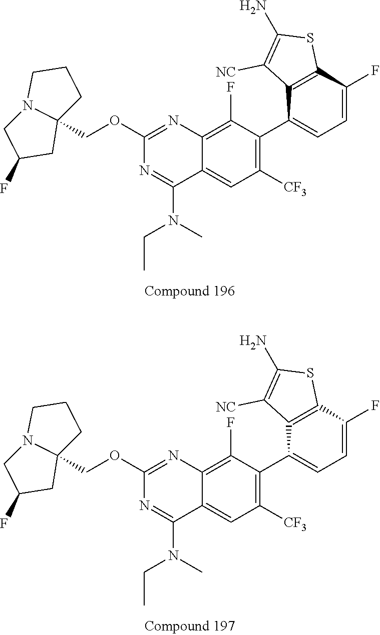

Step A: Preparation of 7-bromo-2-chloro-N-ethyl-8-fluoro-N-methyl-6-(trifluoromethyl)quinazolin-4-amine: To a mixture of 7-bromo-2,4-dichloro-8-fluoro-6-(trifluoromethyl)quinazoline (1.00 eq, 4.00 g, 11.0 mmol) and TEA (3.00 eq, 4.6 mL, 33.0 mmol) in DCM (2 mL) was added N-methylethanamine (1.00 eq, 0.96 mL, 11.0 mmol) in DCM (1 mL) dropwise at −40° C. under N₂. After addition, the resulting solution was stirred at 25° C. for 16 h. The reaction was quenched by water and extracted with DCM (100 mL×3). The combined organic phase was washed with brine, dried over sodium sulfate and concentrated. The residue was purified by column chromatography on silica gel eluted with (PE/EA=100:1-10:1) to afford 7-bromo-2-chloro-N-ethyl-8-fluoro-N-methyl-6-(trifluoromethyl)quinazolin-4-amine (3.22 g, 7.91 mmol, 71.99% yield) as a yellow solid. LCMS ESI (+) m/z 385.8 (M+H)

Step B: Preparation of 7-bromo-N-ethyl-8-fluoro-2-[[(2R,8S)-2-fluoro-1,2,3,5,6,7-hexahydropyrrolizin-8-yl]methoxy]-N-methyl-6-(trifluoromethyl)quinazolin-4-amine: A mixture of 7-bromo-2-chloro-N-ethyl-8-fluoro-N-methyl-6-(trifluoromethyl)quinazolin-4-amine (1.00 eq, 3.26 g, 8.43 mmol), [(2R,8S)-2-fluoro-1,2,3,5,6,7-hexahydropyrrolizin-8-yl]methanol (2.00 eq, 2.69 g, 16.9 mmol) and DIPEA (30.0 eq, 45 mL, 253 mmol) was stirred at 100° C. for 16 h under nitrogen atmosphere. The reaction was quenched by water and extracted with EA (50 mL×4). The organic phase was washed with brine, dried over sodium sulfate and concentrated. The residue was purified by column chromatography on silica gel eluted with (DCM/MeOH=100:1-20:1) to afford 7-bromo-N-ethyl-8-fluoro-2-[[(2R,8S)-2-fluoro-1,2,3,5,6,7-hexahydropyrrolizin-8-yl]methoxy]-N-methyl-6-(trifluoromethyl)quinazolin-4-amine (2.88 g, 4.81 mmol, 57.00% yield) as a yellow solid. LCMS ESI (+) m/z 509 (M+H).

Step C: Preparation of tert-butyl N-[3-cyano-4-[4-[ethyl(methyl)amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophen-2-yl]carbamate: A mixture of 7-bromo-N-ethyl-8-fluoro-2-[[(2R,8S)-2-fluoro-1,2,3,5,6,7-hexahydropyrrolizin-8-yl]methoxy]-N-methyl-6-(trifluoromethyl)quinazolin-4-amine (1.00 eq, 1.83 g, 3.59 mmol), tert-butyl N-[3-cyano-4-(5,5-dimethyl-1,3,2-dioxaborinan-2-yl)-7-fluoro-benzothiophen-2-yl]carbamate (1.50 eq, 2.18 g, 5.39 mmol), Cs₂CO₃ (3.00 eq. 3.51 g, 10.8 mmol) and Pd(DPEPhos)Cl₂ (0.200 eq, 514 mg, 0.719 mmol) in 1,4-dioxane (50 mL) was stirred at 90° C. for 16 h under N₂. The reaction was quenched by water and extracted with EA (30 mL×3). The combined organic phase was washed with brine, dried over sodium sulfate and concentrated. The residue was purified by column chromatography on silica gel eluted with (PE/EA=100:1-1:1) to afford tert-butyl N-[3-cyano-4-[4-[ethyl(methyl)amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophen-2-yl]carbamate (2.14 g, 2.38 mmol, 66.11% yield) as a yellow solid. LCMS ESI (+) m/z 721.1 (M+H).

Step D: Preparation of 2-amino-4-[4-[ethyl(methyl)amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (Compound 81), 2-amino-4-[4-[ethyl(methyl)amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (Compound 196), and 2-amino-4-[4-[ethyl(methyl)amino]-8-fluoro-2-[[(2R,8S)-2-fluoro-1,2,3,5,6,7-hexahydropyrrolizin-8-yl]methoxy-6-(trifluoromethyl)quinazolin-7-yl]-7-fluoro-benzothiophene-3-carbonitrile (Compound 197): A solution of tert-butyl N-[3-cyano-4-[4-[ethyl(methyl)amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophen-2-yl]carbamate (1.00 eq, 2.14 g, 2.38 mmol) and TFA (17.5 eq, 3.2 mL, 41.5 mmol) in DCM (16 mL) was stirred at 25° C. for 16 h. The mixture was concentrated and dried in vacuum to give the crude. The crude was adjusted to pH=8 with saturation sodium bicarbonate aqueous solution, and extracted with EA: THF 2:1 (100 mL×4). The combined organic phase was washed with brine, dried over sodium sulfate and concentrated. The residue was purified by column chromatography on silica gel eluted with (DCM/MeOH=100:1-10:1) to give 2-amino-4-[4-[ethyl(methyl)amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (Compound 81) (1.30 g, 2.09 mmol). LCMS ESI (+) m/z 621.3 (M+H). ¹H NMR (400 MHz, CD₃OD) δ 8.32 (s, 1H), 7.22-7.19 (m, 1H), 7.03-6.90 (m, 1H), 5.44 (s, 0.5H), 5.31 (s, 0.5H), 4.42-4.31 (m, 2H), 3.96-3.87 (m, 3H), 3.51 (s, 3H), 3.46-3.37 (m, 2H), 3.16-3.09 (m, 1H), 2.40-2.16 (m, 3H), 2.14-2.02 (m, 2H), 1.98-1.90 (m, 1H), 1.44 (t, 3H), 2-amino-4-[4-[ethyl(methyl)amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (Compound 81) was separated with chiral chromatography (ChiralPak AD-H 2 cm×25 cm, 5 μm, Hex:EtOH=50:50 (0.1% DEA in Hex)) to give one atropisomer, 2-amino-4-[4-[ethyl(methyl)amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (Compound 196), (341 mg, 0.545 mmol, 22.94% yield) as the second peak; LCMS m/z [M+1]: 621.0. ¹H NMR (400 MHz, CD₃OD) δ 8.29 (s, 1H), 7.22-7.18 (m, 1H), 7.00 (t, 1H), 5.35 (s, 0.5H), 5.22 (s, 0.5H), 4.31-4.19 (m, 2H), 3.96-3.82 (m, 2H), 3.62 (s, 3H), 3.24-3.13 (m, 3H), 3.02-2.96 (m, 1H), 2.36-2.11 (m, 3H), 1.98-1.85 (m, 3H), 1.44 (t, 3H); and the other atropisomer, 2-amino-4-[4-[ethyl(methyl)amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (Compound 197), (263 mg, 0.421 mmol, 17.74% yield) as the first peak: LCMS m/z [M+1]: 621.0. ¹H NMR (400 MHz, CD₃OD) δ 8.50 (s, 1H), 7.22-7.19 (m, 1H), 7.00 (t, 1H), 5.36 (s, 0.5H), 5.22 (s, 0.5H), 4.31-4.20 (m, 2H), 3.93-3.82 (m, 2H), 3.49 (s, 3H), 3.22-3.17 (m, 3H), 3.03-2.97 (m, 1H), 2.38-2.10 (m, 3H), 2.03-1.82 (m, 3H), 1.44 (t, 3H).

### Synthetic Example 11: Synthesis of 2-amino-7-fluoro-4-[8-fluoro-2-[[(2R,8S)-2-fluoro-1,2,3,5,6,7-hexahydropyrrolizin-8-yl]methoxy]-4-[1[(S,2S)-2-(hydroxymethyl)cyclopentyl]-methyl-amino]-6-(trifluoromethyl)quinazolin-7-yl]benzothiophene-3-carbonitrile (Compound 86)

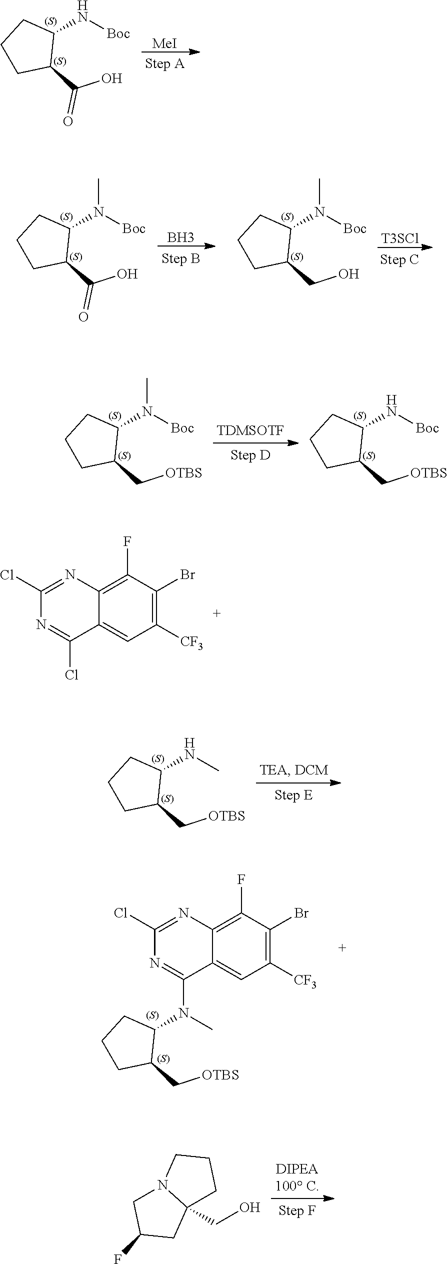

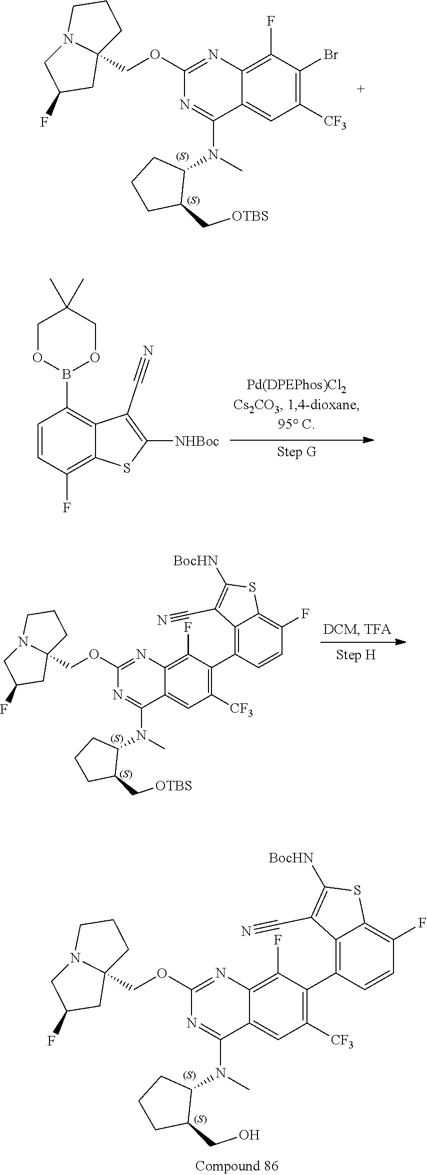

Step A: Preparation of (1S,2S)-2-[tert-butoxycarbonyl(methyl)amino]cyclopentanecarboxylic acid: A solution of (1S,2S)-2-(tert-butoxycarbonylamino)cyclopentanecarboxylic acid (1.00 eq, 1.00 g, 4.36 mmol) in THF (16 mL) was added NaH (6.00 eq, 1.0 g, 26.2 mmol), and stirred at 0° C. for 0.5 hours. Then MeI (3.00 eq, 0.82 mL, 13.1 mmol) was added and stirred at 25° C. for 16 h. It was neutralized with 1N HCl to pH=4 at 0° C., and extracted with EA (10 mL×2). The organic layer was washed with water and brine, dried over Na₂SO₄, concentrated to give crude (1S,2S)-2-[tert-butoxycarbonyl(methyl)amino]cyclopentanecarboxylic acid (1.1 g, 4.07 mmol, 93.2% yield) as a yellow oil. LCMS ESI (+) nm/z 144.2 (M+H−100).

Step B: Preparation of tert-butyl N-[(1S,2S)-2-(hydroxymethyl)cyclopentyl]-N-methyl-carbamate: The solution of (1S,2S)-2-[tert-butoxycarbonyl(methyl)amino]cyclopentanecarboxylic acid (1.00 eq, 1.10 g, 4.52 mmol) in THF (20 mL) was added borane-tetrahydrofuran complex (2.88 eq, 13 mL, 13.0 mmol) at 0° C., and then the mixture was stirred at 65° C. for 2 h. The mixture was cooled to 0° C. and MeOH (20 mL) was added. The mixture was stirred at 65° C. for 1 hour. The mixture was concentrated, and the residue was dissolved in DCM (50 mL). The organic layer was washed with water and brine, dried over Na₂SO₄ and concentrated to give crude tert-butyl N-[(1S,2S)-2-(hydroxymethyl)cyclopentyl]-N-methyl-carbamate (1 g, 3.48 mmol, 77.1% yield) as yellow oil. ¹H NMR (400 MHz, CDCl₃) δ 4.24 (s, 1H), 3.57-3.51 (in, 3H), 2.74 (s, 3H), 1.80-1.60 (m, 7H), 1.43 (s, 911).

Step C: Preparation of tert-butyl N-(1S,2S)-2-[tert-butyl(dimethyl)silyl]oxymethyl]cyclopentyli-N-methyl-carbamate: To a solution of tert-butyl N-[(1S,2S)-2-(hydroxymethyl)cyclopentyl-N-methyl-carbamate (1.00 eq, 1.00 g, 4.36 mmol) in DMF (10 mL) was added TBSCl (2.00 eq, 1.31 g, 8.72 mmol) and imidazole (2.00 eq, 0.59 g, 8.72 mmol). The mixture was stirred at 50° C. for 16 hours. The mixture was quenched with water (20 mL) and extracted with MTBE (10 mL×2). The organic layer was washed with water and brine, dried over Na₂SO₄, concentrated to give crude tert-butyl N-[(1S,2S)-2-[[tert-butyl(dimethyl)silyl]oxymethyl]cyclopentyl]-N-methyl-carbamate (1.6 g, 3.73 mmol, 85.4% yield) as a yellow solid. ¹H NMR (400 MHz, CDCl₃) δ 4.11 (s, 1H), 3.63 (s, 1H), 3.42 (s, 1H), 2.56 (s, 3H), 1.98 (s, 1H), 1.76 (d, J=7.2 Hz, 2H), 1.56 (d, J=7.2 Hz, 4H), 1.42 (d, J=1.2 Hz, 9H), 0.85-0.83 (m, 9H), 0.07-0.02 (m, 6H).

Step D: Preparation of (1S,2S)-2-[[tert-butyl(dimethyl)silyl]oxymethyl]-N-methyl-cyclopentanamine: To a solution of tert-butyl N-[(1S,2S)-2-[[tert-butyl(dimethyl)silyl]oxymethyl]cyclopentyl]-N-methyl-carbamate (1.00 eq, 1.70 g, 4.95 mmol) in DCM (20 mL) was added TBDMSOTf (1.50 eq, 1.67 g, 7.42 mmol) and 2,6-lutidine (2.00 eq, 1.2 mL, 9.90 mmol). The mixture was stirred at 25° C. for 1 hour. The mixture was quenched with water (20 mL) and extracted with DCM (10 mL×2). The combined organic layers were washed with water (20 mL), brine (20 mL), dried over Na₂SO₄ and concentrated. The residue was purified by column chromatography on silica gel (DCM/MeOH=100:1) to afford (1S,2S)-2-[[tert-butyl(dimethyl)silyl]oxymethyl]-N-methyl-cyclopentanamine (870 mg, 3.04 mmol, 61.39% yield) as yellow oil. ¹H NMR (400 MHz, CDCl₃) δ 3.65-3.61 (m, 1H), 3.42-3.38 (m, 1H), 2.90-2.85 (m, 1H), 2.47 (s, 3H), 2.08-1.99 (m, 1H), 1.94-1.50 (m, 5H), 1.28-1.19 (m, 1H), 0.83 (s, 9H), 0.05 (s, 6H).

Step E: Preparation of 7-bromo-N-[(1S,2S)-2-[[tert-butyl(dimethyl)silyl]oxymethyl]cyclopentyl]-2-chloro-8-fluoro-N-methyl-6-(trifluoromethyl)quinazolin-4-amine: To a solution of 7-bromo-2,4-dichloro-8-fluoro-6-(trifluoromethyl)quinazoline (1.00 eq. 500 mg, 1.37 mmol) in DCM (10 mL) were added (1S,2S)-2-[[tert-butyl(dimethyl)silyl]oxymethyl]-N-methyl-cyclopentanamine (1.00 eq, 334 mg, 1.37 mmol) and TEA (3.00 eq, 417 mg, 4.12 mmol). The mixture was stirred at −30° C. for 1 hour. The reaction mixture was poured into water (30 mL) and extracted with DCM (3×10 mL). The combined organic layers were washed with water (20 mL), and brine (10 mL), dried over Na₂SO₄ and concentrated. The residue was purified by prep-TLC (P/E=10/1) to give 7-bromo-N-[(1S,2S)-2-[[tert-butyl(dimethyl)silyl]oxymethyl]cyclopentyl]-2-chloro-8-fluoro-N-methyl-6-(trifluoromethyl)quinazolin-4-amine (440 mg, 0.655 mmol, 47.6% yield) as a yellow solid. LCMS ESI (+) m/z 571.1 (M+H).

Step F: Preparation of 7-bromo-N-[(1S,2S)-2-[[tert-butyl(dimethyl)silyl]oxymethyl]cyclopentyl]-8-fluoro-2-[[(2R,8S)-2-fluoro-1,2,3,5,6,7-hexahydropyrrolizin-8-yl]methoxy]-N-methyl-6-(trifluoromethyl)quinazolin-4-amine: To a solution of 7-bromo-N-[(1S,2S)-2-[[tert-butyl(dimethyl)silyl]oxymethyl]cyclopentyl]-2-chloro-8-fluoro-N-methyl-6-(trifluoromethyl)quinazolin-4-amine (1.00 eq, 490 mg, 0.858 mmol) in DIEA (39.3 eq, 6.0 mL, 33.7 mmol) was added [(2R,8S)-2-fluoro-1,2,3,5,6,7-hexahydropyrrolizin-8-yl]methanol (2.50 eq, 342 mg, 2.15 mmol). The mixture was stirred at 100° C. for 18 hours. The mixture was cooled to 25° C. The reaction mixture was poured into water (10 mL) and extracted with EA (3×5 mL). The combined organic layers were washed with water (10 mL), and brine (10 mL), dried over Na₂SO₄ and concentrated. The residue was purified by prep-TLC (DCM/MeOH=10/1) to give 7-bromo-N-[(1S,2S)-2-[[tert-butyl(dimethyl)silyl]oxymethyl]cyclopentyl]-8-fluoro-2-[[(2R,8S)-2-fluoro-1,2,3,5,6,7-hexahydropyrrolizin-8-yl]methoxy]-N-methyl-6-(trifluoromethyl)quinazolin-4-amine (450 mg, 0.551 mmol, 64.2% yield) as a yellow solid. LCMS ESI (+) m/z 693.2 (M+H).

Step G: Preparation of tert-butyl N-[4-[4-[[(1S,2S)-2-[[tert-butyl(dimethyl)silyl]oxymethyl]cyclopentyl]-methyl-amino]-8-fluoro-2-[[(2R,8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate: To a solution of 7-bromo-N-[(1S,2S)-2-[[tert-butyl(dimethyl)silyl]oxymethyl]cyclopentyl −8-fluoro-2-[[(2R,8S)-2-fluoro-1,2,3,5,6,7-hexahydropyrrolizin-8-yl]methoxy]-N-methyl-6-(trifluoromethyl)quinazolin-4-amine (1.00 eq, 450 mg, 0.649 mmol) in 1,4-dioxane (10 mL) were added tert-butyl N-[3-cyano-4-(5,5-dimethyl-1,3,2-dioxaborinan-2-yl)-7-fluoro-benzothiophen-2-yl]carbamate (1.50 eq, 393 mg, 0.973 mmol), Cs₂CO₃ (3.00 eq, 634 mg, 1.95 mmol) and Pd(DPEphos)Cl₂ (0.500 eq, 0.32 mL, 0.324 mmol). The mixture was stirred at 90° C. under N₂ for 3 hours. The mixture was cooled to 25° C. The reaction mixture was poured into water (10 mL) and extracted with EA (3×5 mL). The combined organic layers were washed with water (10 mL) and brine (10 mL), dried over Na₂SO₄ and concentrated. The residue was purified by prep-TLC (D/M=10/1) to give crude tert-butyl N-[4-[4-[[(1S.2S)-2-[[tert-butyl(dimethyl)silyl]oxymethyl]cyclopentyl]-methyl-amino]-8-fluoro-2-[[(2R,8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate (350 mg, 0.208 mmol, 32.18% yield) as a yellow solid. LCMS ESI (+) m/z 905.3 (M+H).

Step H: Preparation of 2-amino-7-fluoro-4-[8-fluoro-2-[[(2R,8S)-2-fluoro-1,2,3,5,6,7-hexahydropyrrolizin-8-yl]methoxy]-4-[[(1S,2S)-2-(hydroxymethyl)cyclopentyl]-methyl-amino]-6-(trifluoromethyl)quinazolin-7-yl]benzothiophene-3-carbonitrile: To a solution of tert-butyl N-[4-[4-[[(1S,2S)-2-[[tert-butyl(dimethyl)silyl]oxymethyl]cyclopentyl]-methyl-amino]-8-fluoro-2-[[(2R,8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate (1.00 eq, 100 mg, 0.110 mmol) in DCM (4 mL) was added TFA (235 eq, 2.0 mL, 26.0 mmol). The mixture was stirred at 25° C. for 2 hours. The mixture was neutralized with NaHCO₃ aqueous solution to pH=8 at 0° C., and extracted with DCM (3×5 mL). The organic layer was washed with water and brine, dried over Na₂SO₄ and concentrated in vacuo. Then the residue was purified by prep-HPLC and dried by lyophilization to give 2-amino-7-fluoro-4-[8-fluoro-2-[[(2R,8S)-2-fluoro-1,2,3,5,6,7-hexahydropyrrolizin-8-yl]methoxy]-4-[[(1S,2S)-2-(hydroxymethyl)cyclopentyl]-methyl-amino]-6-(trifluoromethyl)quinazolin-7-yl]benzothiophene-3-carbonitrile (20.9 mg, 0.030 mmol, 27.2% yield) as a white solid. MS (ESI), [M+H]+=691.3. ¹H NMR (400 MHz, CD₃OD) δ 8.35 (d, J=6.4 Hz, 1H), 7.24-7.21 (m, 1H), 7.04-7.00 (m, 1H), 5.55 (d, J=3.6 Hz, 0.54H), 5.42 (s, 0.53H), 4.98-4.93 (m, 1H), 4.65-4.49 (m, 2H), 3.88-3.53 (m, 5H), 3.42 (s, 3H), 2.60-1.63 (m, 14H), 1.61-1.52 (m, 1H).

### Synthetic Example 12: Synthesis of 2-amino-7-fluoro-4-[8-fluoro-2-[[(2R,8S)-2-fluoro-1,2,3,5,6,7-hexahydropyrrolizin-8-yl]methoxy]-4-morpholino-6-(trifluoromethyl) quinazolin-7-yl]benzothiophene-3-carbonitrile (Compound 90), 2-amino-7-fluoro-4-[8-fluoro-2-[[(2R,8S)-2-fluoro-1,2,3,5,6,7-hexahydropyrrolizin-8-yl]methoxy]-4-morpholino-6-(trifluoromethyl) quinazolin-7-yl]benzothiophene-3-carbonitrile (Compound 198), and 2-amino-7-fluoro-4-[8-fluoro-2-[[(2R,8S)-2-fluoro-1,2,3,5,6,7-hexahydropyrrolizin-8-yl]methoxy]-4-morpholino-6-(trifluoromethyl) quinazolin-7-yl]benzothiophene-3-carbonitrile (Compound 199)

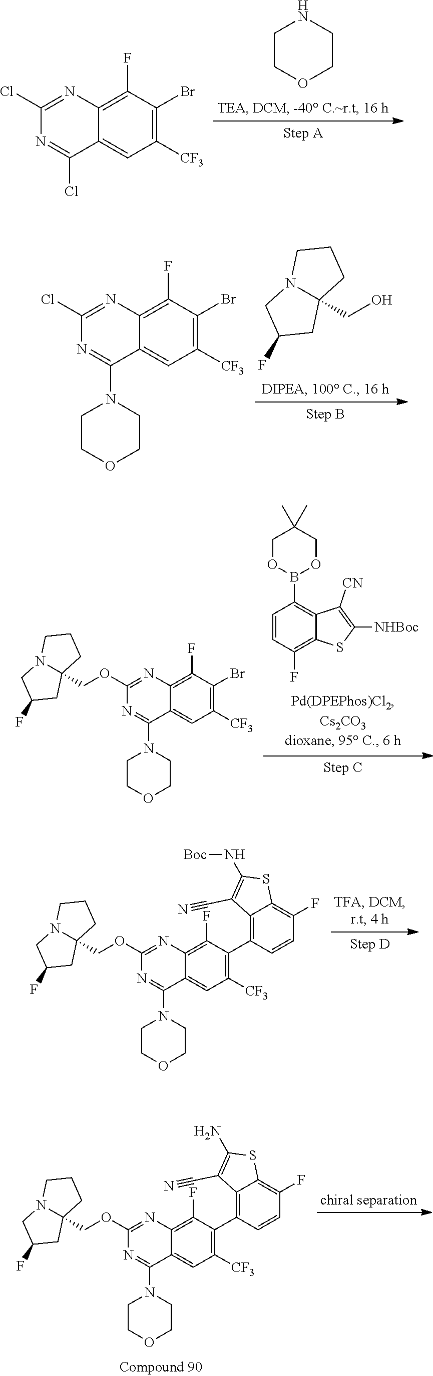

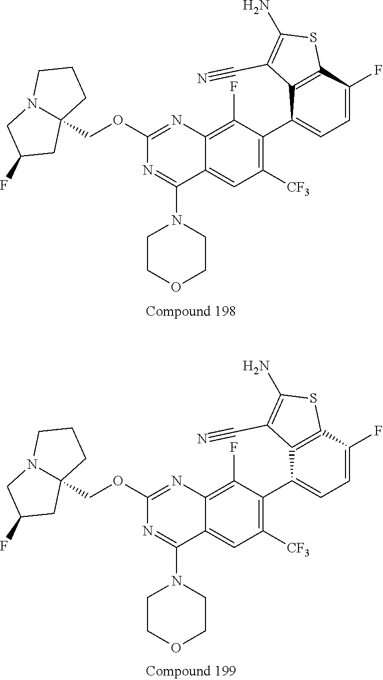

Step A: Preparation of 4-[7-bromo-2-chloro-8-fluoro-6-(trifluoromethyl)quinazolin-4-yl]morpholine: To a solution of 7-bromo-2,4-dichloro-8-fluoro-6-(trifluoromethyl)quinazoline (1.00 eq, 4.5 g, 12.4 mmol) in DCM (20 mL) was added TEA (3.00 eq, 3.754 g, 37.1 mmol), and then morpholine (1.00 eq, 1.077 g, 12.4 mmol) was added dropwise at −40° C. under N₂. After addition the reaction mixture was stirred at 25° C. for 16 h. The mixture was diluted with water and extracted with DCM (4×20 mL). The combined extracts were washed with brine, dried over sodium sulfate, filtered, and concentrated. The residue was purified by silica gel column chromatography eluting with EA in petroleum from 0% to 20% to afford 4-[7-bromo-2-chloro-8-fluoro-6-(trifluoromethyl)quinazolin-4-yl]morpholine (4.30 g, 8.92 mmol, 72.14% yield) as a yellow solid. LCMS ESI (+) m/z 413.8 (M+H).

Step B: Preparation of 4-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]morpholine: A solution of 4-[7-bromo-2-chloro-8-fluoro-6-(trifluoromethyl)quinazolin-4-yl]morpholine (1.00 eq, 4.3 g, 10.4 mmol) and [(2R,8S)-2-fluoro-1,2,3,5,6,7-hexahydropyrrolizin-8-yl]methanol (2.00 eq, 3.302 g, 20.7 mmol) in DIEA (30.0 eq, 55 mL, 311 mmol) was stirred at 100° C. for 16 h under nitrogen atmosphere. The reaction was concentrated, diluted with water and extracted with EA (5×50 mL). Tho combined extracts were washed with brine, dried over sodium sulfate, filtered, and concentrated. The residue was purified by silica gel column chromatography eluting with MeOH in DCM from 0.5% to 2% to afford 4-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]morpholine (3.00 g, 4.19 mmol, 40.37% yield) as a yellow solid. LCMS ESI (+) m/z 536.9 (M+II).

Step C: Preparation of tert-butyl N-[3-cyano-7-fluoro-4-[8-fluoro-2-[[(2R,8S)-2-fluoro-1,2,3,5,6,7-hexahydropyrrolizin-8-yl]methoxy]-4-morpholino-6-(trifluoromethyl)quinazolin-7-yl]benzothiophen-2-yl]carbamate: A solution of 4-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]morpholine (1.00 eq, 3.00 g, 5.58 mmol), tert-butyl N-[3-cyano-4-(5,5-dimethyl-1,3,2-dioxaborinan-2-yl)-7-fluoro-benzothiophen-2-yl]carbamate (1.50 eq, 3.386 g, 8.37 mmol), Cs₂CO₃ (3.00 eq, 5.4 g, 16.7 mmol) and DPEPhosPdCl₂ (0.400 eq, 1.599 g, 2.23 mmol) in 1,4-dioxane (150 mL) was stirred under nitrogen at 95° C. for 6 h under N₂. The reaction was monitored by LCMS. After the reaction completed, the mixture was then diluted with water and extracted with EA (4×100 mL). The combined extracts were washed with brine, dried over sodium sulfate, filtered and concentrated. The residue was purified by silica gel column chromatography eluting with EA in petroleum from 0% to 30% to afford tert-butyl N-[3-cyano-7-fluoro-4-[8-fluoro-2-[[(2R,8S)-2-fluoro-1,2,3,5,6,7-hexahydropyrrolizin-8-yl]methoxy]-4-morpholino-H(trifluoromethyl)quinazolin-7-yl]benzothiophen-2-yl]carbamate (2.50 g, 2.00 mmol, 35.88% yield) as a yellow solid. LCMS ESI (+) m/z 749.1 (M+H).

Step D: Preparation of 2-amino-7-fluoro-4-[8-fluoro-2-1[(2R,8S)-2-fluoro-1,2,3,5,6,7-hexahydropyrrolizin-8-yl]methoxy]-4-morpholino-6-(trifluoromethyl)quinazolin-7-yl]benzothiophene-3-carbonitrile (Compound 90), 2-amino-7-fluoro-4-[8-fluoro-2-[[(2R,8S)-2-fluoro-1,2,3,5,6,7-hexahydropyrrolizin-8-yl]methoxy]-4-morpholino-6-(trifluoromethyl)quinazolin-7-yl]benzothiophene-3-carbonitrile (Compound 198), and 2-amino-7-fluoro-4-[8-fluoro-2-[[(2R,8S)-2-fluoro-1,2,3,5,6,7-hexahydropyrrolizin-8-yl]methoxy]-4-morpholino-6-(trifluoromethyl)quinazolin-7-yl]benzothiophene-3-carbonitrile (Compound 199): A solution of tert-butyl N-[3-cyano-7-fluoro-4-[8-fluoro-2-[[(2R,8S)-2-fluoro-1,2,3,5,6,7-hexahydropyrrolizin-8-yl]methoxy]-4-morpholino-6-(trifluoromethyl)quinazolin-7-yl]benzothiophen-2-yl]carbamate (1.00 eq, 2.5 g, 2.00 mmol) and TFA (64.8 eq, 10 mL, 130 mmol) in DCM (30 mL) was stirred at 25° C. for 4 h. The reaction solution was adjusted to pH=8 with saturated sodium bicarbonate aqueous solution, extracted with EA (50 mL×4). The combined organic phase was washed with brine, dried over sodium sulfate and concentrated. The residue was purified by silica gel column chromatography eluting with MeOH in DCM from 0.5% to 10% to give crude product.

The crude product was then purified by prep-TLC to give 2-amino-7-fluoro-4-[8-fluoro-2-[[(2R,8S)-2-fluoro-1,2,3,5,6,7-hexahydropyrrolizin-8-yl]methoxy]-4-morpholino-6-(trifluoromethyl)quinazolin-7-yl]benzothiophene-3-carbonitrile (Compound 90) (550 mg, 0.85 mmol). LCMS m/z [M+1]: 649.0. ¹H NMR (400 MHz, CDOD) δ 8.19 (S, 1H), 7.20-7.23 (m, 1H), 7.02 (t, 1H), 5.62 (s, 0.5H), 5.48 (s, 0.5H), 4.59-4.75 (m, 2H), 3.82-4.09 (m, 11H), 3.43-3.47 (m, 1H), 2.15-2.72 (m, 6H). 2-Amino-7-fluoro-4-[8-fluoro-2-[[(2R,8S)-2-fluoro-1,2,3,5,6,7-hexahydropyrrolizin-8-yl]methoxy]-4-morpholino-6-(trifluoromethyl) quinazolin-7-yl]benzothiophene-3-carbonitrile (Compound 90) was separated with chiral chromatography (ChiralPak AD-H 2 cm×25 cm, 5 μm, Hex:EtOH=50:50 (0.1% DEA in Hex)) to give one atropisomer 2-amino-7-fluoro-4-[8-fluoro-2-[[(2R,8S)-2-fluoro-1,2,3,5,6,7-hexahydropyrrolizin-8-yl]methoxy]-4-morpholino-6-(trifluoromethyl)quinazolin-7-yl]benzothiophene-3-carbonitrile (Compound 198) (171 mg, 0.260 mmol, 12.98% yield) as second peak; LCMS m/z [M+1]: 649.0 (M+H), ¹H NMR (400 MHz, DMSO) δ 8.09 (d, 3H), 7.23-7.26 (m, 1H), 7.13 (t, 1H), 5.35 (s, 0.5H), 5.21 (s, 0.5H), 4.15 (d, 1H), 4.05 (d, 1H), 3.93-3.96 (m, 2H), 3.78-3.88 (m, 6H), 3.03-3.11 (m, 3H), 2.82-2.84 (m, 1H), 2.01-2.16 (m, 3H), 1.77-1.86 (m, 3H); and give the other isomer 2-amino-7-fluoro-4-[8-fluoro-2-[[(2R,8S)-2-fluoro-1,2,3,5,6,7-hexahydropyrrolizin-8-yl]methoxy]-4-morpholino-6-(trifluoromethyl)quinazolin-7-yl]benzothiophene-3-carbonitrile (Compound 199) (175 mg, 0.264 mmol, 13.16% yield) as the first peak; LCMS m/z [M+1]: 649.0. ¹H NMR (400 MHz, DMSO) δ 8.09 (d, 3H), 7.23-7.26 (m, 1H), 7.13 (t, 1H), 5.34 (s, 0.5H), 5.21 (s, 0.5H), 4.14 (d, 1H), 4.06 (d, 1H), 3.93-3.96 (m, 2H), 3.78-3.82 (m, 6H), 3.02-3.10 (m, 3H), 2.82-2.83 (m, 1H), 2.01-2.15 (m, 3H), 1.77-1.85 (m, 3H).

### Synthetic Example 13: Synthesis of 2-amino-4-[4-(1,1-dioxo-1,4-thiazinan-4-yl)-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (Compound 91)

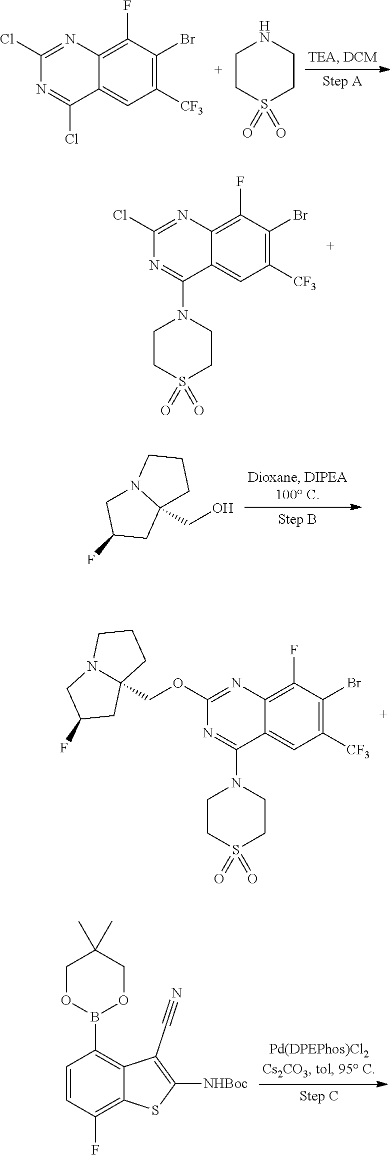

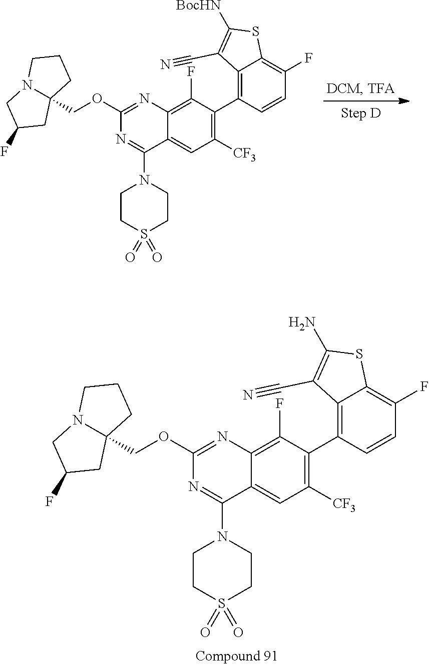 [SMILES: CC(C)(C)OC(=O)Nc1sc2c(F)ccc(-c3c(C(F)(F)F)cc4c(N5CCS(=O)(=O)CC5)nc(OC[C@@]56CCCN5C[C@H](F)C6)nc4c3F)c2c1C#N ; N#Cc1c(N)sc2c(F)ccc(-c3c(C(F)(F)F)cc4c(N5CCS(=O)(=O)CC5)nc(OC[C@@]56CCCN5C[C@H](F)C6)nc4c3F)c12]

Step A: Preparation of 4-[7-bromo-2-chloro-8-fluoro-6-(trifluoromethyl)quinazolin-4-yl]-1,4-thiazinane 1,1-dioxide: To a solution of 7-bromo-2,4-dichloro-8-fluoro-6-(trifluoromethyl)quinazoline (1.00 eq, 450 mg, 1.10 mmol) in DCM (10 mL) were added 1,4-thiazinane 1,1-dioxide (1.00 eq, 168 mg, 1.10 mmol) and TEA (3.00 eq, 628 mg, 3.30 mmol). The resulting mixture was stirred at −40° C. for 1 h, then returned to room temperature for 0.5 h. The mixture was then diluted with water and extracted with DCM (3×10 mL). The combined extracts were washed with brine, dried over sodium sulfate, filtered and concentrated. The crude product was purified by silica gel column chromatography eluting with EA in petroleum from 0% to 50% to afford 4-[7-bromo-2-chloro-8-fluoro-6-(trifluoromethyl)quinazolin-4-yl]-1,4-thiazinane 1,1-dioxide (500 mg, 1.04 mmol, 94.39% yield) as a solid. LCMS ESI (+) m/z 461.9 (M+H).

Step B: Preparation of 4-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-1,4-thiazinane 1,1-dioxide: To a solution of 4-[7-bromo-2-chloro-8-fluoro-6-(trifluoromethyl)quinazolin-4-yl]-1,4-thiazinane 1,1-dioxide (1.00 eq, 400 mg, 0.865 mmol) in 1,4-dioxane (5 mL) was added [(2R,8S)-2-fluoro-1,2,3,5,6,7-hexahydropyrrolizin-8-yl]methanol (2.00 eq, 275 mg, 1.73 mmol) and DIPEA (5.00 eq, 0.77 mL, 4.32 mmol). The reaction mixture was stirred at 90° C. for 16 h. The resulting mixture was added to water and extracted with EA (3×10 mL), the organic layer was concentrated under reduced pressure, and the crude product was purified by prep-TLC (DCM:MeOH=20:1) to give 4-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-1,4-thiazinane 1,1-dioxide (170 mg, 0.262 mmol, 30.27% yield) as a solid. LCMS ESI (+) m/z 584.9 (M+H).

Step C: Preparation of tert-butyl N-[3-cyano-4-[4-(1,1-dioxo-1,4-thiazinan-4-yl)-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophen-2-yl]carbamate: To a solution of tert-butyl N-[3-cyano-4-(5,5-dimethyl-1,3,2-dioxaborinan-2-yl)-7-fluoro-benzothiophen-2-yl]carbamate (1.50 eq, 155 mg, 0.384 mmol) in 1,4-dioxane (3 mL) was added 4-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-1,4-thiazinane 1,1-dioxide (1.00 eq, 150 mg, 0.256 mmol), Cs₂CO₃ (3.00 eq, 251 mg, 0.769 mmol) and Pd(DPEPhos)Cl₂ (0.400 eq, 73 mg, 0.102 mmol). The reaction mixture was stirred at 95° C. for 3 hours under N₂. The resulting mixture was added to water and extracted with EA (3×10 mL), the organic layer was concentrated under reduced pressure, and the crude product was purified by prep-TLC (DCM:MeOH=20:1) to give tert-butyl N-[3-cyano-4-[4-(1,1-dioxo-1,4-thiazinan-4-yl)-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophen-2-yl]carbamate (280 mg, 0.0738 mmol, 28.80% yield) as a solid. LCMS ESI (−) m/z 797.1 (M+H).

Step D: Preparation of 2-amino-4-[4-(1,1-dioxo-1,4-thiazinan-4-yl)-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile: To a solution of tert-butyl N-[3-cyano-4-[4-(1,1-dioxo-1,4-thiazinan-4-yl)-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophen-2-yl]carbamate (1.00 eq, 260 mg, 0.326 mmol) in DCM (2 mL) was added TFA (24.7 eq, 0.60 mL, 8.05 mmol) The reaction mixture was stirred at 25° C. for 5 h. The solvent was removed under vacuum, the residue was purified by prep-HPLC to give 2-amino-4-[4-(1,1-dioxo-1,4-thiazinan-4-yl)-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (30 mg, 0.0403 mmol, 12.36% yield) as a white solid. MS (ESI), [M+H]+=697.0. ¹H NMR (400 MHz, CD₃OD) δ 8.25 (s, 1H), 7.23-7.26 (m, 1H), 7.05 (t, 1H), 5.65 (s, 0.5H), 5.52 (s, 0.5H), 4.60-4.81 (m, 2H), 4.39-4.46 (m, 3H), 3.88-4.08 (m, 3H), 3.41-3.58 (m, 4H), 2.33-2.79 (m, 8H).

### Synthetic Example 14: Synthesis of 2-amino-4-(4-(((S)-1,1-dioxidotetrahydrothiophen-3-yl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile (Compound 94)

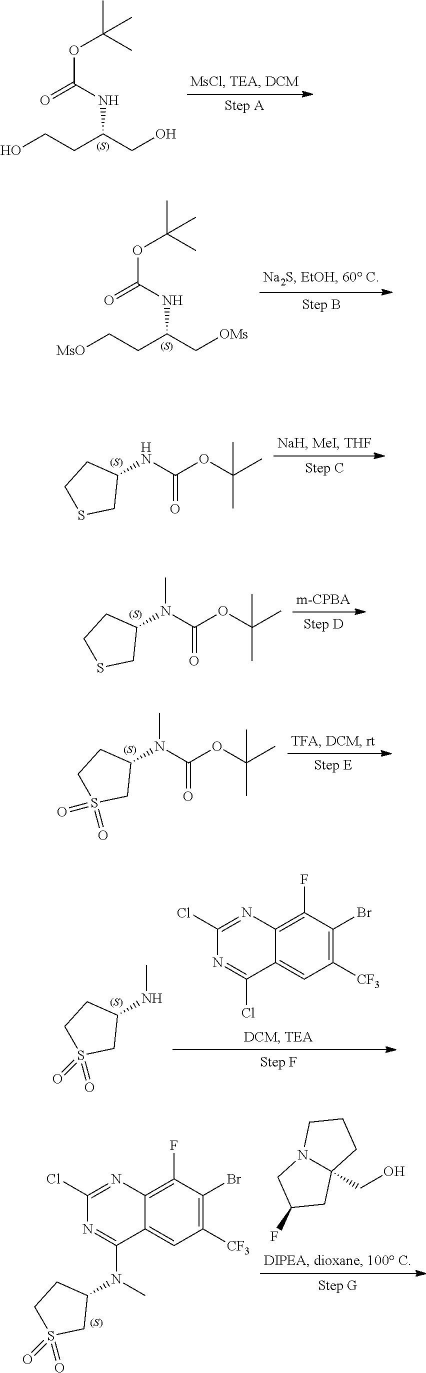

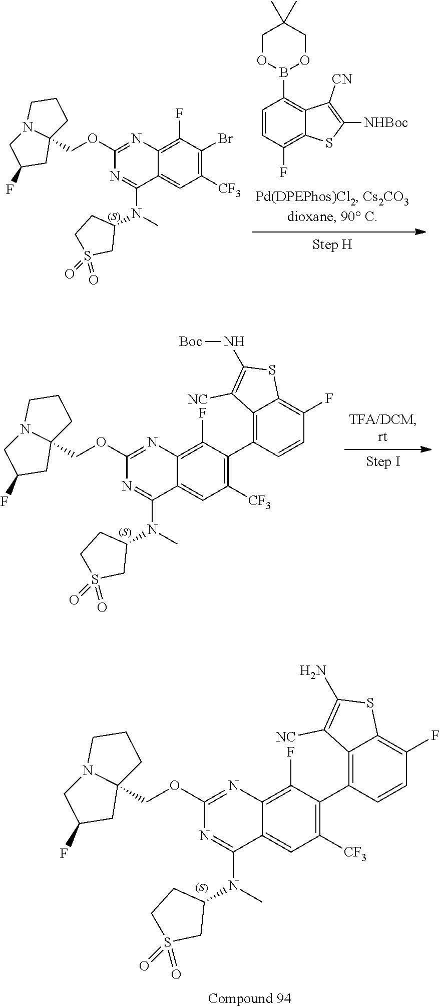

Step A: Preparation of (S)-2-((tert-butoxycarbonyl)amino)butane-1,4-diyl dimethanesulfonate: To a solution of tert-butyl N-[(1S)-3-hydroxy-1-(hydroxymethyl)propyl]carbamate (1.00 eq. 9.00 g, 43.8 mmol) in DCM (100 mL) was added MsCl (3.00 eq, 15 g, 132 mmol) and TEA (6.00 eq, 26.6 g, 263 mmol). The reaction mixture was stirred at 0° C. for 1 h. The reaction was added to water (50 mL) at 0° C. The resulting mixture was extracted with DCM (50 mL×3). The combined organic layers were washed with brine, dried over anhydrous sodium sulfate, filtered and concentrated under reduced pressure to give (S)-2-((tert-butoxycarbonyl)amino)butane-1,4-diyl dimethanesulfonate (10 g, 27.7 mmol, 63.1% yield) as a white solid. LCMS ESI (+) m/z 362 (M+H).

Step B: Preparation of tert-butyl (S)-(tetrahydrothiophen-3-yl)carbamate: To a solution of (S)-2-((tert-butoxycarbonyl)amino)butane-1,4-diyl dimethanesulfonate (1.00 eq, 6.00 g, 16.6 mmol) in ethanol (60 mL) was added Na₂S·9H₂O (1.05 eq, 2.00 g, 17.4 mmol). After addition, the reaction mixture was stirred at 60° C. for 1 h. The reaction was poured into water (40 mL) and extracted with DCM (50 mL×3). The combined organic layers were washed with brine, dried over anhydrous sodium sulfate, filtered, and concentrated under reduced pressure to give product as a yellow oil. The residue was purified by column chromatography (PE/EA=20:1-PE/EA=10:1) to give tert-butyl (S)-(tetrahydrothiophen-3-yl)carbamate (4.6 g, 22.60 mmol, 81.59% yield) as a white solid. LCMS ESI (+) m/z 204 (M+H).

Step C: Preparation of tert-butyl (S)-methyl(tetrahydrothiophen-3-yl)carbamate: To a solution of NaH (3.00 eq, 2.4 g, 59.0 mmol) in THF (50 mL) was added tert-butyl N-[(3S)-tetrahydrothiophen-3-yl]carbamate (1.00 eq, 4.00 g, 19.7 mmol) at 0° C. under nitrogen atmospheric. After addition, the reaction mixture was stirred at 0° C. for 30 min. Then MeI (3.00 eq. 8.38 g, 59.0 mmol) was added dropwise at 0° C. under nitrogen atmosphere. The reaction mixture was stirred at room temperature for 16 h. The reaction mixture was poured into ice water (100 mL) and extracted with EA (40 mL×3). The combined organic layers were washed with brine (50 mL), dried over anhydrous sodium sulfate, filtered and concentrated under reduced pressure to give tert-butyl (S)-methyl(tetrahydrothiophen-3-yl)carbamate (5.5 g, crude) as a yellow oil. LCMS ESI (+) m/z 218 (M+H).

Step D: Preparation of tert-butyl (S)-(1,1-dioxidotetrahydrothiophen-3-yl)(methyl)carbamate: To a solution of tert-butyl (S)-methyl(tetrahydrothiophen-3-yl)carbamate (1.00 eq, 5.00 g, 23.0 mmol) in DCM (50 mL) was added m-CPBA (3.00 eq, 11.91 g, 69.0 mmol) at 0° C. under N₂. After addition, the reaction mixture was stirred at 20° C. for 2 h. The reaction was poured into water (100 mL) and extracted with DCM (50 mL×3). The combined organic layers were washed with brine, dried over anhydrous sodium sulfate, filtered and concentrated. The residue was purified by column chromatography (PE/EA=10:1→PE/EA=3:1) to give tert-butyl (S)-(1,1-dioxidotetrahydrothiophen-3-yl)(methyl)carbamate (3 g, 12.10 mmol, 68.36% yield) as a yellow oil. LCMS ESI (+) m/z 250 (M+H).

Step E: Preparation of (S)-3-(methylamino)tetrahydrothiophene 1,1-dioxide: To a solution of tert-butyl (S)-(1,1-dioxidotetrahydrothiophen-3-yl)(methyl)carbamate (1.00 eq, 2.50 g, 10.0 mmol) in DCM (20 mL) was added TFA (8.03 eq, 6.0 mL, 80.5 mmol). After addition, the reaction mixture was stirred at 25° C. for 2 h. The reaction mixture was concentrated to give (S)-3-(methylamino)tetrahydrothiophene 1,1-dioxide (2 g. crude) as a yellow oil. LCMS ESI (+) m/z 150 (M+H).

Step F: Preparation of (S)-3-((7-bromo-2-chloro-8-fluoro-6-(trifluoromethyl)quinazolin-4-yl)(methyl)amino)tetrahydrothiophene 1,1-dioxide: To a solution of 7-bromo-2,4-dichloro-8-fluoro-6-(trifluoromethyl)quinazoline (1.00 eq, 600 mg, 1.65 mmol) in DCM (5 mL) were added TEA (3.00 eq, 501 mg, 4.95 mmol) and (3S)—N-methyl-1,1-dioxo-thiolan-3-amine (1.00 eq, 246 mg, 1.65 mmol) at −40° C. under N₂. After addition, the reaction mixture was stirred at 0° C. for 2 h. The reaction was poured into water (10 mL) and extracted with DCM (5 mL×3). The combined organic layers were washed with brine, dried over anhydrous sodium sulfate, filtered and concentrated under reduced pressure to give (S)-3-((7-bromo-2-chloro-8-fluoro-6-(trifluoromethyl)quinazolin-4-yl)(methyl)amino)tetrahydrothiophene 1,1-dioxide (400 mg, 0.84 mmol, 50.60% yield) as a yellow solid. LCMS ESI (+) m/z 476 (M+H).

Step G: Preparation of (S)-3-((7-bromo-8-fluoro-2-(((2R,7aS)-2-fluorotetrahydro-1H-pyrrolizin-7a(5H)-yl)methoxy)-6-(trifluoromethyl)quinazolin-4-yl)(methyl)amino)tetrahydrothiophene 1,1-dioxide: To a solution of (S)-3-((7-bromo-2-chloro-8-fluoro-6-(trifluoromethyl)quinazolin-4-yl)(methyl)amino)tetrahydrothiophene 1,1-dioxide (1.00 eq, 300 mg, 0.629 mmol) in DMSO (10 mL) was added [(2R,8S)-2-fluoro-1,2,3,5,6,7-hexahydropyrrolizin-8-yl]methanol (2.00 eq, 200 mg, 1.26 mmol) and potassium fluoride (8.00 eq, 292 mg, 5.03 mmol). The reaction mixture was stirred at 90° C. for 5 h. The reaction was poured into water (30 mL) and extracted with EA (20 mL×3). The combined organic layers were washed with brine, dried over anhydrous sodium sulfate, filtered and concentrated under reduced pressure. The crude was purified by prep-TLC (PE/EA-1:1) to give (S)-3-((7-bromo-8-fluoro-2-(((2R,7aS)-2-fluorotetrahydro-1H-pyrrolizin-7a(5H)-yl)methoxy)-6-(trifluoromethyl)quinazolin-4-yl)(methyl)amino)tetrahydrothiophene 1,1-dioxide (220 mg, 0.37 mmol, 44.05% yield) as a yellow solid. LCMS ESI (+) m/z 599 (M+H).

Step H: Preparation of tert-butyl (3-cyano-4-(4-(((S)-1,1-dioxidotetrahydrothiophen-3-yl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophen-2-yl)carbamate: To a solution of (S)-3-((7-bromo-8-fluoro-2-(((2R,7aS)-2-fluorotetrahydro-1H-pyrrolizin-7a(5H)-yl)methoxy)-6-(trifluoromethyl)quinazolin-4-yl)(methyl)amino)tetrahydrothiophene 1,1-dioxide (1.00 eq, 200 mg, 0.334 mmol) in 1,4-dioxane (5 mL) were added tert-butyl N-[3-cyano-4-(5,5-dimethyl-1,3,2-dioxaborinan-2-yl)-7-fluoro-benzothiophen-2-yl]carbamate (1.50 eq, 202 mg, 0.500 mmol), Cs₂CO, (3.00 eq, 326 mg, 1.00 mmol) and Pd(DPEPhos)Cl₂ (0.500 eq, 120 mg, 0.167 mmol) under N₂. The resulting mixture was stirred at 90° C. for 3 h under N₂. The reaction was poured into water (10 mL) and extracted with EA (10 mL×3). The combined organic layers were washed with brine, dried over anhydrous sodium sulfate, filtered and concentrated under reduced pressure. The crude was purified by prep-TLC (PE/EA=1:1) to give tert-butyl (3-cyano-4-(4-(((S)-1,1-dioxidotetrahydrothiophen-3-yl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophen-2-yl)carbamate (80 mg, 0.10 mmol, 27.03% yield) as a yellow solid. LCMS ESI (+) m/z 599 (M+H)

Step I: Preparation of 2-amino-4-(4-(((S)-1,1-dioxidotetrahydrothiophen-3-yl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile: To a solution of tert-butyl (3-cyano-4-(4-(((S)-1,1-dioxidotetrahydrothiophen-3-yl)(methyl)amino)-8-fluoro-2-(((2R,7aS)-2-fluorotetrahydro-1H-pyrrolizin-7a(5H)-yl)methoxy)-6-(trifluoremethyl)quinazolin-7-yl)-7-fluorobenzo[b]thiophen-2-yl)carbamate (1.00 eq, 50 mg, 0.0617 mmol) in DCM (5 mL) was added TFA (544 eq, 2.5 mL, 33.6 mmol). After addition the reaction mixture was stirred at 20° C. for 1 h. The reaction was added to aqueous sodium bicarbonate solution (10 mL) at 0° C. The resulting mixture was extracted with DCM (10 mL) three times. The combined organic layers were washed with brine (10 mL), and the reaction was concentrated to dryness. The residue was purified by prep-HPLC to give 2-amino-4-(4-(((S)-1,1-dioxidotetrahydrothiophen-3-yl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile (6.3 mg, 0.009 mmol, 9.00% yield) as a white solid. LCMS ESI (+) m/z 711 (M+H). ¹H NMR (400 MHz, CD_{T}OD) δ 8.34 (s, 1H), 7.23-7.20 (m, 1H), 7.04-6.99 (m, 1H), 5.48-5.44 (m, 1.5H), 5.32 (s, 0.5H), 4.46-4.37 (m, 2H), 3.68-3.63 (m, 1H), 3.52 (s, 3H), 3.46-3.36 (m, 4H), 3.26-3.13 (m, 2H), 2.68-1.99 (m, 9H).

### Synthetic Example 15: Synthesis of 4-[4-[1-acetyl-2-methyl-pyrrolidin-3-yl]-methyl-amino]-8-fluoro-2-[[(2R,8S)-2-fluoro-1,2,3,5,6,7-hexahydropyrrolizin-8-yl]methoxy]-6-(trifluoromethyl) quinazolin-7-yl]-2-amino-7-fluoro-benzothiophene-3-carbonitrile (Compound 114), 4-((R)-4-(((2R,3R)-1-acetyl-2-methylpyrrolidin-3-yl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 192), 4-((S)-4-(((2R,3R)-1-acetyl-2-methylpyrrolidin-3-yl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 193), 4-((R)-4-(((2S,3S)-1-acetyl-2-methylpyrrolidin-3-yl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 194), and 4-((S)-4-(((2S,3S)-1-acetyl-2-methylpyrrolidin-3-yl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 195)

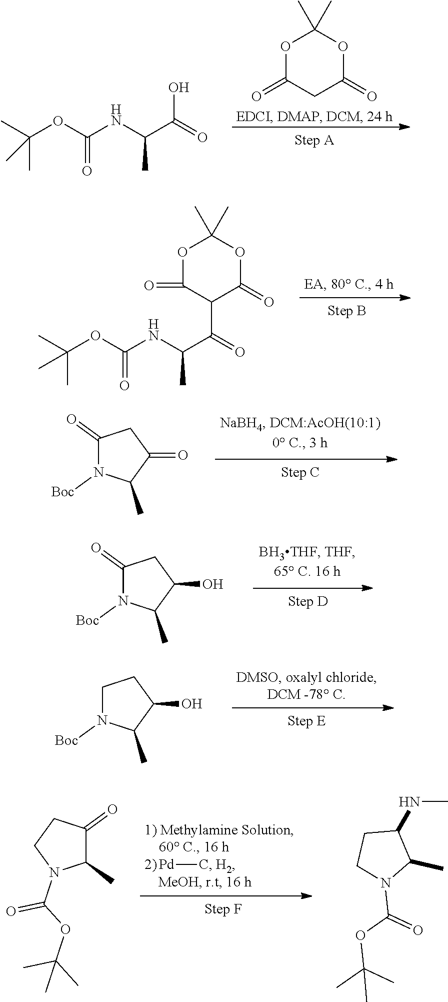

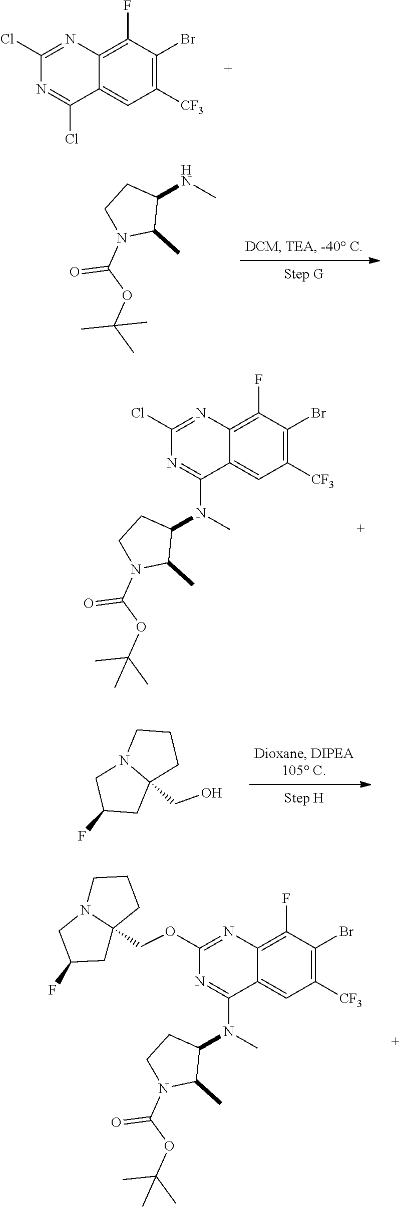

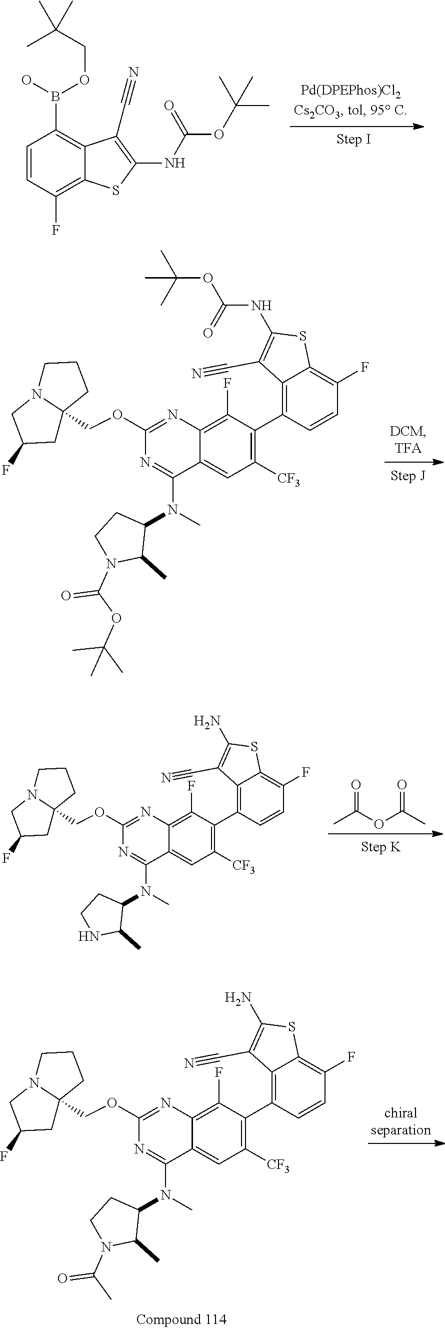

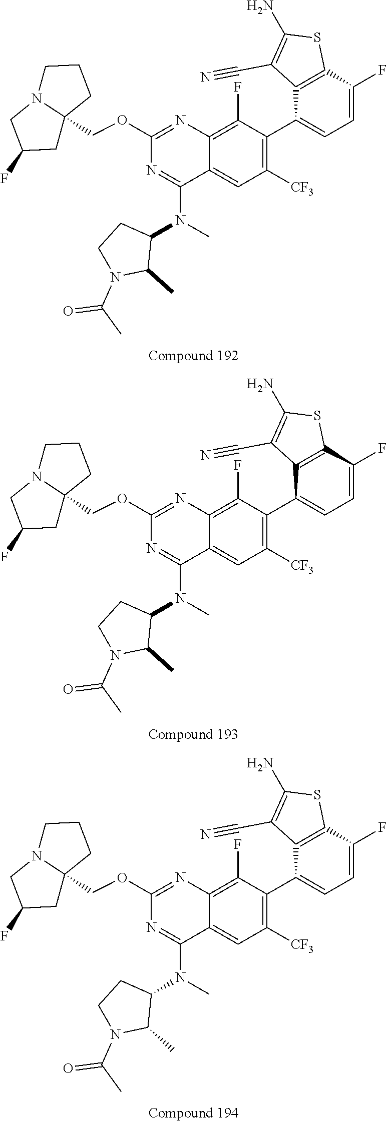

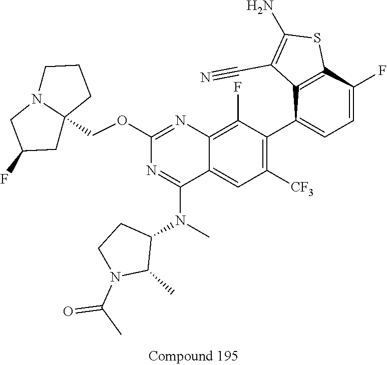

Step A: Preparation of tert-butyl (R)-(1-(2,2-dimethyl-4,6-dioxo-1,3-dioxan-5-yl)-1-oxopropan-2-yl)carbamate: To a solution of 2,2-dimethyl-1,3-dioxane-4,6-dione (1.10 eq, 37.71 g, 262 mmol) in DCM (1000 mL) were added (2R)-2-(tert-butoxycarbonylamino) propanoic acid (1.00 eq, 45.00 g, 238 mmol) and DMAP (1.50 eq, 43.52 g, 357 mmol), and then EDCI (2.40 eq, 109.59 g, 571 mmol) was added at 0° C. After addition the reaction mixture was stirred at 25° C. for 16 hours. LC-MS showed the reaction was complete. The solvent was removed and the residue was dissolved in EA (1000 mL), washed with 5% NaHSO₄ (5×200 mL) and brine (200 mL), and dried over Na₂SO₄ to give a solution of tert-butyl N-[(1R)-2-(2,2-dimethyl-4,6-dioxo-1,3-dioxan-5-yl)-1-methyl-2-oxo-ethyl]carbamate (75.00 g, 190 mmol, 80.01% yield) in EA, which was used in the next step directly without further purification. LCMS ESI (+) m/z 338.2 (M+23)

Step B: Preparation of tert-butyl (R)-2-methyl-3,5-dioxopyrrolidine-1-carboxylate: A mixture of tert-butyl N-[(1R)-2-(2,2-dimethyl-4,6-dioxo-1,3-dioxan-5-yl)-1-methyl-2-oxo-ethyl]carbamate (1.00 eq, 75.00 g, 190 mmol) in ethyl acetate (1000 mL) was stirred at 80° C. for 3 hours. The solvent was removed to give tert-butyl (2R)-2-methyl-3,5-dioxo-pyrrolidine-1-carboxylate (40.00 g, 167 mmol, 87.74% yield) as a yellow solid, which was used in the next step directly without further purification. LCMS ESI (+) m/z 158.1 (M+H−56)

Step C: Preparation of tert-butyl (2R,3R)-3-hydroxy-2-methyl-5-oxopyrrolidine-1-carboxylate: To a solution of tert-butyl (2R)-2-methyl-3,5-dioxo-pyrrolidine-1-carboxylate (1.00 eq, 43.00 g, 202 mmol) in DCM (1000 mL) was added acetic acid (100 mL), and then NaBH₄ (2.00 eq. 15.33 g, 403 mmol) was added slowly in portions at 0° C. under N₂. After addition the reaction mixture was stirred at 0° C. for 3 hours. The reaction mixture was adjusted to pH-8 with sat. NaHCO₃. The resulting mixture was extracted with EA (3×300 mL), the combined organic phase was washed with brine, dried over Na₂SO₄, and concentrated. The residue was purified by silica gel (PE/EA=1/1) to give tert-butyl (2R,3R)-3-hydroxy-2-methyl-5-oxo-pyrrolidine-1-carboxylate (20.00 g, 88.3 mmol, 43.77% yield) as a white solid. ¹H NMR (400 MHz, CDCl₃) δ 4.52-4.47 (m, 1H), 4.29-4.23 (m, 1H), 2.61-2.55 (m, 2H), 2.23 (d, 1H), 1.53 (s, 9H), 1.33 (d, 3H).

Step D: Preparation of tert-butyl (2R,3R)-3-hydroxy-2-methylpyrrolidine-1-carboxylate: To a solution of tert-butyl (2R,3R)-3-hydroxy-2-methyl-5-oxo-pyrrolidine-1-carboxylate (1.00 eq, 20.00 g, 92.9 mmol) in THF (200 mL) was added 1M BH₃/THF (3.00 eq, 280 mL, 279 mmol) at 0° C., and after addition the reaction mixture was stirred at 65° C. for 16 hours. LC-MS showed the reaction was complete. The reaction mixture was quenched with NH₄Cl solution, extracted with EA (3×100 mL). The combined organic phase was washed with brine, dried over Na₂SO₄, and concentrated. The residue was purified by column chromatography on silica gel (PE/EA=2/1) to give tert-butyl (2R,3R)-3-hydroxy-2-methyl-pyrrolidine-1-carboxylate (15.00 g, 70.8 mmol, 76.20% yield) as a white solid. LCMS ESI (+) m/z 146.1 (M+H−56). ¹H NMR (400 MHz, CDCl₃) δ 4.33-4.30 (m, 1H), 3.86 (s, 1H), 3.41-3.35 (m, 2H), 2.05-2.02 (m, 1H), 1.87-1.82 (m, 1H), 1.67 (d, 1H), 1.49 (s, 9H), 1.20 (d, 3H).

Step E: Preparation of tert-butyl (R)-2-methyl-3-oxopyrrolidine-1-carboxylate: To a solution of DMSO (3.00 eq, 11.63 g, 149 mmol) in DCM (50 mL) was added oxalyl chloride (2.00 eq, 12.62 g, 99.4 mmol) at −78° C. under N₂. The mixture was stirred at −78° C. for 1 h, and then a solution of tert-butyl (2R,3R)-3-hydroxy-2-methyl-pyrrolidine-1-carboxylate (1.00 eq, 10.00 g, 49.7 mmol) in DCM (50 mL) was added and stirred at −78° C. for 1 h. TEA (6.00 eq, 30.17 g, 298 mmol) was added and stirred at-78° C. for 1 h. The mixture was then diluted with water and extracted with DCM (3×100 mL). The combined extracts were washed with brine, dried over sodium sulfate, filtered and concentrated. The residue was purified by column chromatography on silica gel to give tert-butyl (R)-2-methyl-3-oxopyrrolidine-1-carboxylate (8.00 g, 36.1 mmol, 72.73% yield) as a yellow oil. ¹H NMR (400 MHz, CDCl₃) δ 3.91 (t, 2H), 3.61-3.54 (m, 1H), 2.65-2.49 (m, 2H), 1.49 (s, 9H), 1.32 (d, 3H).

Step F: Preparation of tert-butyl 2-methyl-3-(methylamino)pyrrolidine-1-carboxylate: A solution of tert-butyl (2R)-2-methyl-3-oxo-pyrrolidine-1-carboxylate (1.00 eq, 5.00 g, 25.1 mmol) in methylamine ethanol solution (30.9 eq, 100 mL, 774 mmol) was stirred at 25° C. for 16 h. The solvent was removed under vacuum, the residue was dissolved in methanol, Pd/C (1.00 eq, 2671 mg, 25.1 mmol) was added and the mixture was stirred at 15° C. under H₂ for 16 h, then filtered and concentrated. The residue was purified by column chromatography on silica gel to give tert-butyl 2-methyl-3-(methylamino)pyrrolidine-1-carboxylat (3.30 g, 13.9 mmol, 55.23% yield) as a yellow oil. LCMS ESI (+) m/z 215 (M+H). ¹H NMR (400 MHz, CDCl₃) δ 4.06-3.93 (m, 1H), 3.39-3.13 (m, 3H), 2.44 (d, 3H), 2.07-2.04 (m, 1H), 1.62-1.52 (m, 1H), 1.46 (s, 9H), 1.02 (d, 3H).

Step G: Preparation of tert-butyl 3-[[7-bromo-2-chlor-8-fluoro-6-(trifluoromethyl) quinazolin-4-yl]-methyl-aminol-2-methyl-pyrrolidine-1-carboxylate: To a solution of 7-bromo-2,4-dichloro-8-fluoro-6-(trifluoromethyl)quinazoline (1.00 eq, 155 mg, 0.426 mmol) in DCM (2 mL) was added TEA (3.00 eq, 129 mg, 1.278 mmol), then tert-butyl 2-methyl-3-(methylamino)pyrrolidine-1-carboxylate (1.00 eq, 91 mg, 0.426 mmol) in DCM (2 mL) and was stirred at-40° C. under N₂. After addition the reaction mixture was stirred at 25° C. for 16 h. The reaction was quenched with ice-water, extracted with DCM (3×20 mL), and the combined organic phase was washed with brine, dried over Na₂SO₄, and concentrated. The residue was purified by prep-TLC to afford tert-butyl 3-[[7-bromo-2-chloro-8-fluoro-6-(trifluoromethyl)quinazolin-4-yl]-methyl-amino]-2-methyl-pyrrolidine-1-carboxylate (226 mg, 0.396 mmol, 93.05% yield) as a yellow solid. LCMS ESI (+) m/z 541 (M+H).

Step H: Preparation of tert-butyl 3-[[7-bromo-8-fluoro-2-[(2R,8S)-2-fluoro-1,2,3,5,6,7-hexahydropyrrolizin-8-yl]methoxy]-6-(trifluoromethyl)quinazolin-4-yl]-methyl-amino]-2-methyl-pyrrolidine-1-carboxylate: A solution of tert-butyl 3-[[7-bromo-2-chloro-8-fluoro-6-(trifluoromethyl)quinazolin-4-yl]-methyl-amino]-2-methyl-pyrrolidine-1-carboxylate (1.00 eq, 206 mg, 0.380 mmol), [(2R,8S)-2-fluoro-1,2,3,5,6,7-hexahydropyrrolizin-8-yl]methanol (2.00 eq, 121 mg, 0.760 mmol) and DIEA (30.0 eq, 2.0 mL, 11.4 mmol) was stirred at 100° C. for 16 h. The reaction was quenched with water and extracted with EA (4×50 mL). The combined organic phase was washed with brine, dried over sodium sulfate and concentrated. The residue was purified by prep-TLC to give tert-butyl 3-[[7-bromo-8-fluoro-2-[[(2R,8S)-2-fluoro-1,2,3,5,6,7-hexahydropyrrolizin-8-yl]methoxy]-6-(trifluoromethyl) quinazolin-4-yl]-methyl-amino]-2-methyl-pyrrolidine-1-carboxylate (169 mg, 0.234 mmol, 61.53% yield) as a yellow solid. LCMS ESI (+) m/z 664 (M+H).

Step I: Preparation of tert-butyl 3-[[7-[2-(tert-butoxycarbonylamino)-3-cyano-7-fluoro-benzothiophen-4-yl]-8-fluoro-2-[[(2R,8S)-2-fluoro-1,2,3,5,6,7-hexahydropyrrolizin-8-yl]methoxy]-6-(trifluoromethyl)quinazolin-4-yl]-methyl-aminol-2-methyl-pyrrolidine-1-carboxylate: To a mixture of tert-butyl 3-[[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-methyl-amino]-2-methyl-pyrrolidine-1-carboxylate (1.00 eq, 149 mg, 0.224 mmol) in 1,4-dioxane (5 mL) were added tert-butyl N-[3-cyano-4-(5,5-dimethyl-1,3,2-dioxaborinan-2-yl)-7-fluoro-benzothiophen-2-yl]carbamate (1.50 eq, 136 mg, 0.336 mmol), potassium trimethylsilanolate (3.00 eq, 0.14 mL, 0.673 mmol) and Pd(DPEPhos)Cl₂ (0.400 eq, 0.088 mL, 0.0897 mmol), and after addition the reaction mixture was stirred at 95° C. for 16 h under N₂. The reaction was quenched by water and extracted by EA (3×30 mL). The combined organic phase was washed with brine, dried over sodium sulfate and concentrated. The residue was purified by prep-TIC to give tert-butyl 3-[[7-[2-(tert-butoxycarbonylamino)-3-cyano-7-fluoro-benzothiophen-4-yl]-8-fluoro-2-[[(2R,8S)-2-fluoro-1,2,3,5,6,7-hexahydropyrrolizin-8-yl]methoxyl-6-(trifluoromethyl)quinazolin-4-yl]-methyl-amino]-2-methyl-pyrrolidine-1-carboxylate (97 mg, 0.0554 mmol, 24.69% yield) as a yellow solid. LCMS ESI (+) m/z 876 (M+H).

Step J: Preparation of 2-amino-7-fluoro-4-[8-fluoro-2-[[(2R,8S)-2-fluoro-1,2,3,5,6,7-hexahydropyrrolizin-8-yl]methoxy]-4-[methyl-[2-methylpyrrolidin-3-yl]amino]-6-(trifluoromethyl)quinazolin-7-yl]benzothiophene-3-carbonitrile: A solution of tert-butyl 3-[[7-[2-(tert-butoxycarbonylamino)-3-cyano-7-fluoro-benzothiophen-4-yl]-8-fluoro-2-[[(2R,8S)-2-fluoro-1,2,3,5,6,7-hexahydropyrrolizin-8-yl]methoxy]-6-(trifluoromethyl)quinazolin-4-yl]-methyl-amino]-2-methyl-pyrrolidine-1-carboxylate (1.00 eq, 87 mg, 0.0993 mmol) and TFA (65.3 eq, 0.50 mL, 6.49 mmol) in DCM (2 mL) was stirred at 25° C. for 4 h. The mixture was concentrated and dried in vacuum. The residue was purified by prep-HPLC to give 2-amino-7-fluoro-4-[8-fluoro-2-[[(2R,8S)-2-fluoro-1,2,3,5,6,7-hexahydropyrrolizin-8-yl]methoxy]-4-[methyl-[2-methylpyrrolidin-3-yl]amino]-6-(trifluoromethyl)quinazolin-7-yl]benzothiophene-3-carbonitrile (33 mg, 0.0464 mmol, 46.71% yield) as a white solid. LCMS ESI (+) m/z 676 (M+II).

Step K: Preparation of 4-[4-[I-acetyl-2-methyl-pyrrolidin-3-yl]-methyl-amino]-8-fluoro-2-[[(2R,8S)-2-fluoro-1,2,3,5,6,7-hexahydropyrrolizin-8-yl]methoxy]-6-(trifluoromethyl)quinazolin-7-yl]-2-amino-7-fluoro-benzothiophene-3-carbonitrile (Compound 114), 4-((R)-4-(((2R,3R)-1-acetyl-2-methylpyrrolidin-3-yl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 192), 4-((S)-4-(((2R,3R)-1-acetyl-2-methylpyrrolidin-3-yl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 193), 4-((R)-4-(((2S,3S)-1-acetyl-2-methylpyrrolidin-3-yl)methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 194), and 4-((S)-4-(((2S,3S)-1-acetyl-2-methylpyrrolidin-3-yl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 195): To a solution of 2-amino-7-fluoro-4-[8-fluoro-2-[[(2R,8S)-2-fluoro-1,2,3,5,6,7-hexahydropyrrolizin-8-yl]methoxy]-4-[methyl-[2-methylpyrrolidin-3-yl]amino]-6-(trifluoromethyl)quinazolin-7-yl]benzothiophene-3-carbonitrile (1.00 eq, 1500 mg, 2.2 mmol) in DCM (20 mL) was added TEA (5.00 eq, 11 mmol) at −30° C. under N₂, then a solution of acetic anhydride (1.0 eq, 0.21 mL, 2.2 mmol) was added dropwise at-30° C. After addition the reaction mixture was stirred at −30° C. for 1 h, the reaction was quenched by MeOH and water, and extracted with DCM (3×5 mL). The combined organic phase % as washed with brine, dried over sodium sulfate and concentrated. The residue was purified by column chromatography to give 4-[4-[1-acetyl-2-methyl-pyrrolidin-3-yl]-methyl-amino]-8-fluoro-2-1[(2R,8S)-2-fluoro-1,2,3,5,6,7-hexahydropyrrolizin-8-yl]methoxy]-6-(trifluoromethyl)quinazolin-7-yl]-2-amino-7-fluoro-benzothiophene-3-carbonitrile (Compound 114) (580 mg, 0.81 mmol, 37% yield) as a white solid. LCMS ESI (+) m/z 718 (M+H). ¹H NMR (400 MHz, CD₃OD) δ 8.39 (s, 1H), 7.25 (t, 1H), 7.04 (t, 1H), 5.53-5.53 (m, 1H), 5.02-4.98 (m, 1H), 4.62-4.44 (m, 2H), 3.65-3.62 (m, 6H), 3.53-3.50 (m, 1H), 3.25-3.19 (m, 2H), 2.48-2.43 (m, 2H), 2.17 (s, 2H), 2.11 (s, 3H), 1.33 (m, 5H), 1.18-1.17 (m, 1H), 1.11-1.07 (m, 2H), 4-[4-[1-acetyl-2-methyl-pyrrolidin-3-yl]-methyl-amino]-8-fluoro-2-[[(2R,8S)-2-fluoro-1,2,3,5,6,7-hexahydropyrrolizin-8-yl]methoxy]-6-(trifluoromethyl) quinazolin-7-yl]-2-amino-7-fluoro-benzothiophene-3-carbonitrile (Compound 114) was separated with chiral chromatography ChiralPak AD-H 2 cm×25 cm, 5 μm, Hex EtOH (0.1% DEA in hexane)) to give two fractions. The first fraction, which contained two atropisomers, was further separated by (ChiralPak IG-H 2 cm×25 cm, 5 μm, Hex:EtOH (0.1% TFA in hexane) to give 4-((R)-4-(((2R,3R)-1-acetyl-2-methylpyrrolidin-3-yl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 192), as the first peak, (129 mg, 0.18 mmol): LCMS m/z [M+1]: 718.1. ¹H NMR (400 MHz, CD₃OD) δ 8.41 (s, 1H), 7.22 (t, 1H), 7.03 (t, 1H), 5.62 (s, 0.5H), 5.49 (s, 0.5H), 4.98 (t, 1H), 4.83-4.66 (m, 3H), 4.02-3.80 (m, 4H), 3.71-3.44 (m, 5H), 2.77-2.58 (m, 3H), 2.49-2.30 (m, 4H), 2.20-2.06 (m, 4H), 1.18-1.07 (m, 3H); and 4-((R)-4-(((2S,3S)-1-acetyl-2-methylpyrrolidin-3-yl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 194), as the second peak, (111 mg, 0.155 mmol) LCMS m/z [M+1]: 718.1. ¹H NMR (400 MHz, CD₃OD) δ 8.41 (s, 1H), 7.24 (t, 1H), 7.03 (t, 1H), 5.62 (s, 0.5H), 5.49 (s, 0.5H), 5.00 (t, 1H), 4.80-4.63 (m, 3H), 4.05-3.81 (m, 4H), 3.71-3.65 (m, 4H), 3.49-3.43 (m, 1H), 2.76-2.56 (m, 3H), 2.49-2.30 (m, 4H), 2.16-2.09 (m, 4H), 1.17-1.05 (m, 3H). The second fraction, which contained two other atropisomers, was further separated by ChiralPak AD-H 2 cm×25 cm, 5 μm, Hex:EtOH (0.1% TFA in hexane)) to give 4-((S)-4-(((2R,3R)-1-acetyl-2-methylpyrrolidin-3-yl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 193) (100 mg, 0.14 mmol), as the first peak, LCMS m/z [M+1]: 718.1. ¹H NMR (400 MHz, CD₃OD) δ 8.41 (s, 1H), 7.22 (t, 1H), 7.03 (t, 1H), 5.63 (s, 0.5H), 5.50 (s, 0.5H), 5.00 (t, 11H), 4.83-4.60 (m, 31H), 4.08-3.81 (m, 41H), 3.77-3.57 (m, 4H), 3.48-3.42 (m, 1H), 2.77-2.58 (m, 3H), 2.49-2.30 (m, 4H), 2.20-2.03 (m, 4H), 1.19-1.07 (m, 3H); and 4-((S)-4-(((2S,3S)-1-acetyl-2-methylpyrrolidin-3-yl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 195), as the second peak, (107 mg, 0.15 mmol): LCMS m/z [M+1]: 718.1. ¹H NMR (400 MHz, CD₃OD) δ 8.41 (s, 1H), 7.23 (t, 1H), 7.03 (t, 1H), 5.62 (s, 0.5H), 5.49 (s, 0.5H), 4.98 (t, 1H), 4.81-4.58 (m, 3H), 4.04-3.80 (m, 4H), 3.71-3.56 (m, 4H), 3.49-3.42 (m, 1H), 2.73-2.57 (m, 3H), 2.49-2.30 (m, 4H), 2.14-2.09 (m, 4H), 1.16-1.05 (m, 31H).

### Synthetic Example 16: 2-amino-7-fluoro-4-[8-fluoro-2-[[(2S,8R)-2-fluoro-1,2,3,5,6,7-hexahydropyrrolizin-8-yl]methoxy]-4-(6-oxa-3-azabicyclo[3.1.1]heptan-3-yl)-6-(trifluoromethyl)quinazolin-7-yl]benzothiophene-3-carbonitrile (Compound 145)

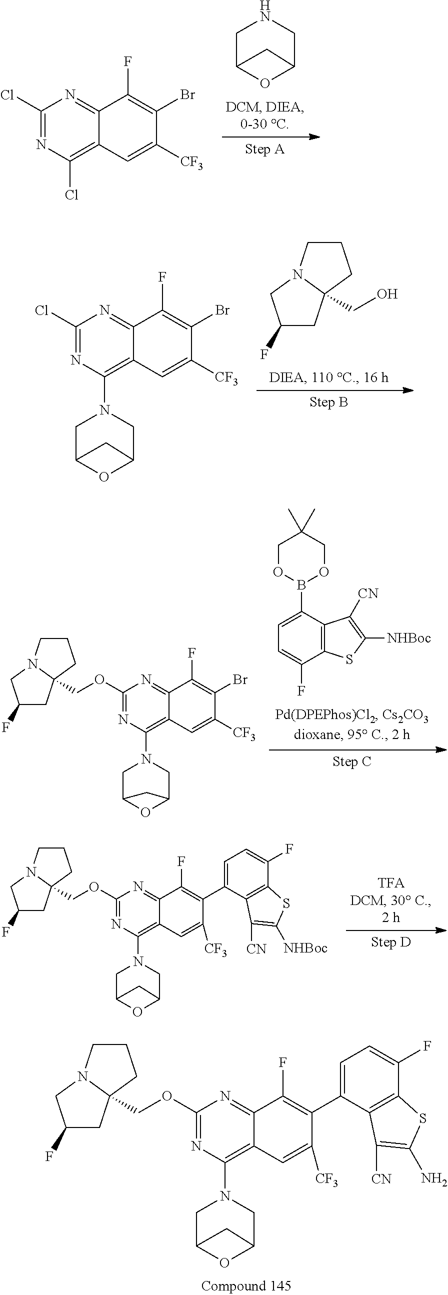

Step A: Preparation of 3-[7-bromo-2-chloro-8-fluoro-6-(trifluoromethyl)quinazolin-4-yl]-6-oxa-3-azabicyclo[3.1.1]heptane: To a mixture of 7-bromo-2,4-dichloro-8-fluoro-6-(trifluoromethyl)quinazoline (1.00 eq, 223 mg, 0.613 mmol) in DCM (2 mL) were added DIEA (3.98 eq, 0.43 mL, 2.44 mmol) and 6-oxa-3-azabicyclo[3.1.1]heptane (1.65 eq, 100 mg, 1.01 mmol) in DCM at 0° C., and the mixture was stirred at 30° C. for 2 hrs. LCMS detected the desired product. The reaction was concentrated to dryness and the residue was taken up in EtOAc (30 mL) and the organic layers washed with 2×30 mL saturated brine solution. The organic layers were dried (Na₂SO₄) before concentration to dryness, and the crude was then purified by Prep-TLC (EtOAc/PE=1:4) to get 3-[7-bromo-2-chloro-8-fluoro-6-(trifluoromethyl)quinazolin-4-yl]-6-oxa-3-azabicyclo[3.1.1]heptane (200 mg, 0.469 mmol, 76.51% yield). LCMS ESI (+) m/z 425.8 (M+H).

Step B: Preparation of 3-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-6-oxa-3-azabicyclo[3.1.1]heptane: To a mixture of 3-[7-bromo-2-chloro-8-fluoro-6-(trifluoromethyl)quinazolin-4-yl]-6-oxa-3-azabicyclo[3.1.1]heptane (1.00 eq, 180 mg, 0.422 mmol) in DIEA (3.0 mL) was added [(2R,8S)-2-fluoro-1,2,3,5,6,7-hexahydropyrrolizin-8-yl]methanol (4.00 eq, 269 mg, 1.69 mmol), and the mixture was stirred at 110° C. for 16 hrs. LCMS detected the desired product. The reaction was concentrated to dryness, the residue was taken up in EtOAc (50 mL) and the organic layers washed with 2×30 mL saturated brine solution. The organic layers were dried (Na₂SO₄) before concentration to dryness, and the crude was purified by Prep-TLC(EtOAc/PE=1:1) to give 3-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-6-oxa-3-azabicyclo[3.1.1]heptane (90 mg, 0.164 mmol, 38.83% yield). LCMS ESI (+) m/z 550.0 (M+H).

Step C: Preparation of tert-butyl N-[3-cyano-7-fluoro-4-[8-fluoro-2-[[(2R,8S)-2-fluoro-1,2,3,5,6,7-hexahydropyrrolizin-8-yl]methoxy]-5-(6-oxa-3-azabicyclo[3.1.1]heptan-3-yl)-6-(trifluoromethyl)quinazolin-7-yl]benzothiophen-2-yl]carbamate: To a solution of 3-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-6-oxa-3-azabicyclo[3.1.1]heptane (1.00 eq, 90 mg, 0.164 mmol) in 1,4-dioxane (2 mL) were added Cs₂CO₃ (3.00 eq, 160 mg, 0.492 mmol), tert-butyl N-[3-cyano-4-(5,5-dimethyl-1,3,2-dioxaborinan-2-yl)-7-fluoro-benzothiophen-2-yl]carbamate (3.00 eq, 199 mg, 0.492 mmol) and DPEPhosPdCl₂ (0.400 eq, 47 mg, 0.0655 mmol). The mixture was bubbled with Ar for 1-2 min and sealed, and the mixture was stirred for 2 hrs at 95° C. The mixture extracted with EtOAc (20 mL×3) and the combined organic layers were concentrated to dryness under vacuum. The residue was purified by Prep-TLC (DCM/MeOH=20/1) to give tert-butyl N-[3-cyano-7-fluoro-4-[8-fluoro-2-[[(2R,8S)-2-fluoro-1,2,3,5,6,7-hexahydropyrrolizin-8-yl]methoxy]-5-(6-oxa-3-azabicyclo[3.1.1]heptan-3-yl)-6-(trifluoromethyl)quinazolin-7-yl]benzothiophen-2-yl]carbamate (55 mg, 0.0723 mmol, 44.13% yield). LCMS ESI (+) m/z 761.0 (M+H).

Step D: Preparation of 2-amino-7-fluoro-4-[8-fluoro-2-[[(2S,8R)-2-fluoro-1,2,3,5,6,7-hexahydropyrrolizin-8-yl]methoxy]-4-(6-oxa-3-azabicyclo[3.1.1]heptan-3-yl)-6-(trifluoromethyl)quinazolin-7-yl]benzothiophene-3-carbonitrile: To a solution of tert-butyl N-[3-cyano-7-fluoro-4-[8-fluoro-2-[[(2S,8R)-2-fluoro-1,2,3,5,6,7-hexahydropyrrolizin-8-yl]methoxy]-4-(6-oxa-3-azabicyclo[3.1.1]heptan-3-yl)-6-(trifluoromethyl)quinazolin-7-yl]benzothiophen-2-yl]carbamate (1.00 eq, 55 mg, 0.0723 mmol) in DCM (2 mL) was added TFA (180 eq, 1.0 mL, 13.0 mmol), and the mixture was stirred for 2 hrs at 30° C. The organic layer was concentrated to dryness under vacuum, and the residue was purified by Prep-HPLC to give 2-amino-7-fluoro-4-[8-fluoro-2-[[(2S,8R)-2-fluoro-1,2,3,5,6,7-hexahydropyrrolizin-8-yl]methoxy]-4-(6-oxa-3-azabicyclo[3.1.1]heptan-3-yl)-6-(trifluoromethyl)quinazolin-7-yl]benzothiophene-3-carbonitrile (14 mg, 0.0214 mmol, 29.63% yield).

LCMS ESI (+) m/z 661.0 (M+H). ¹H NMR (400 MHz, CD₃OD) δ 8.63 (s, 1H), 7.20-7.24 (m, 1H), 7.02 (t, J=9.2 Hz, 1H), 5.43 (d, J=62.88 Hz, 1H), 4.43-4.59 (m, 5H), 4.30-4.38 (m, 2H), 3.47-3.78 (m, 3H), 1.95-2.56 (m, 8H), 1.29 (s, 2H).

### Synthetic Example 17: 4-(4-(((2S,3R)-1-acetyl-2-methylpyrrolidin-3-yl)((methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 190)

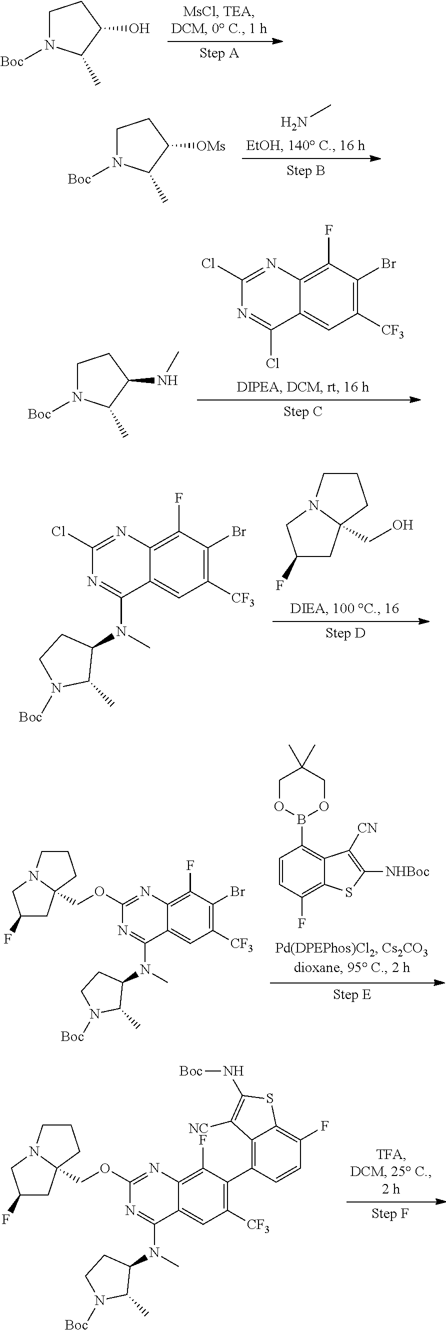

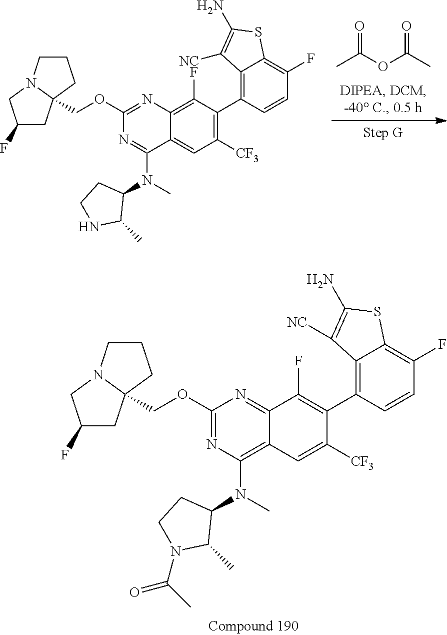

Step A: Preparation of tert-butyl (2S,3S)-2-methyl-3-((methylsulfonyl)oxy)pyrrolidine-1-carboxylate: To a solution of tert-butyl (2S,3S)-3-hydroxy-2-methyl-pyrrolidine-1-carboxylate (1.00 eq, 1.00 g, 4.97 mmol) and TEA (3.00 eq, 1.51 g, 14.9 mmol) in DCM (10 mL) was added MsCl (1.20 eq, 0.68 g, 5.96 mmol) at ° C. under N₂. The mixture was stirred at 0° C. for 1 h. The mixture was then diluted with water and extracted with DCM (3×20 mL). The combined extracts were washed with brine, dried over sodium sulfate, filtered and concentrated to give crude tert-butyl (2S,3S)-2-methyl-3-methylsulfonyloxy-pyrrolidine-1-carboxylate (1.20 g, 3.44 mmol, 69.16% yield) as a yellow oil. LCMS ESI (+) m/z 224.0 (M+H−56).

Step B: Preparation of tert-butyl (2S,3R)-2-methyl-3-(methylamino)pyrrolidine-1-carboxylate: A solution of tert-butyl (2S,3S)-2-methyl-3-methylsulfonyloxy-pyrrolidine-1-carboxylate (1.00 eq, 1200 mg, 4.30 mmol) in monomethylamine ethanol solution (200 eq, 26.6 g, 859 mmol) was stirred at 140° C. for 16 h under nitrogen atmosphere. The solvent was removed and the residue was purified by column chromatography on silica gel to give tert-butyl (2S,3R)-2-methyl-3-(methylamino)pyrrolidine-1-carboxylate (300 mg, 1.12 mmol, 26.07% yield) as yellow oil. LCMS ESI (+) m/z 215.1 (M+H).

Step C: Preparation of tert-butyl (2S,3R)-3-((7-bromo-2-chloro-8-fluoro-6-(trifluoromethyl)quinazolin-4-yl)(methyl)amino)-2-methylpyrrolidine-1-carboxylate: To a solution of 7-bromo-2,4-dichloro-8-fluoro-6-(trifluoromethyl)quinazoline (1.00 eq, 600 mg, 1.65 mmol) and DIEA (5.00 eq, 1.5 mL, 8.24 mmol) in DCM (10 mL) was added tert-butyl (2S,3R)-2-methyl-3-(methylamino)pyrrolidine-1-carboxylate (1.00 eq, 353 mg, 1.65 mmol) at −30° C. under N₂. After addition the mixture was stirred at 25° C. for 16 h. The mixture was then diluted with water and extracted with DCM (3×20 mL). The combined extracts were washed with brine, dried over sodium sulfate, filtered and concentrated. The residue was purified by prep-TLC to give tert-butyl (2S,3R)-3-[[7-bromo-2-chloro-8-fluoro-6-(trifluoromethyl)quinazolin-4-yl]-methyl-amino]-2-methyl-pyrrolidine-1-carboxylate (550 mg, 0.964 mmol, 58.50% yield) as a yellow solid. LCMS ESI (+) m/z 540.9 (M+H).

Step D: Preparation of tert-butyl (2S,3R)-3-((7-bromo-8-fluoro-2-(((2R,7aS)-2-fluorotetrahydro-1H-pyrrolizin-7a(5H)-yl)methoxy)-6-(trifluoromethyl)quinazolin-4-yl)(methyl)amino)-2-methylpyrrolidine-1-carboxylate: A solution of tert-butyl (2S,3R)-3-[[7-bromo-2-chloro-8-fluoro-6-(trifluoromethyl)quinazolin-4-yl]-methyl-amino]-2-methyl-pyrrolidine-1-carboxylate (1.00 eq, 550 mg, 1.02 mmol) and [(2R,8S)-2-fluoro-1,2,3,5,6,7-hexahydropyrrolizin-8-yl]methanol (1.00 eq, 162 mg, 1.02 mmol) in DIPEA (5 mL) was stirred at 100° C. for 16 h under atmosphere. The mixture was then diluted with water and extracted with EA (3×20 mL). The combined extracts were washed with brine, dried over sodium sulfate, filtered and concentrated. The residue was purified by prep-TLC to give tert-butyl (2S,3R)-3-[[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-methyl-amino]-2-methyl-pyrrolidine-1-carboxylate (560 mg, 0.801 mmol, 78.86% yield) as a yellow solid. LCMS ESI (+) m/z 664.0 (M+H).

Step E: Preparation of tert-butyl (2S,3R)-3-((7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)(methyl)amino)-2-methylpyrrolidine-1-carboxylate: To a suspension of Cs₂CO₃ (5.00 eq, 1373 mg, 4.21 mmol) in 1,4-dioxane (25 mL) was added a solution of tert-butyl (2S,3R)-3-[[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-methyl-amino]-2-methyl-pyrrolidine-1-carboxylate (1.00 eq, 560 mg, 0.843 mmol), tert-butyl N-[3-cyano-4-(5,5-dimethyl-1,3,2-dioxaborinan-2-yl)-7-fluoro-benzothiophen-2-yl]carbamate (2.00 eq, 681 mg, 1.69 mmol) and Pd(DPEPhos)Cl₂ (0.200 eq, 121 mg, 0.169 mmol) in 1,4-dioxane (25 mL) at 95° C. under N₂. After addition the resulting solution was stirred at 95° C. for 4 h under N₂. The mixture was then diluted with water and extracted with EA (3×20 mL). The combined extracts were washed with brine, dried over sodium sulfate, filtered and concentrated, and the residue was purified by prep-TLC to give tert-butyl (2S,3R)-3-[[7-[2-(tert-butoxycarbonylamino)-3-cyano-7-fluoro-benzothiophen-4-yl]-8-fluoro-2-[[(2R,8S)-2-fluoro-1,2,3,5,6,7-hexahydropyrrolizin-8-yl]methoxy]-6-(trifluoromethyl)quinazolin-4-yl]-methyl-amino]-2-methyl-pyrrolidine-1-carboxylate (580 mg, 0.344 mmol, 40.86% yield) as a yellow solid. LCMS ESI (+) m/z 876.3 (M+H)

Step F: Preparation of 2-amino-7-fluoro-4-(8-fluoro-2-(((2R,7aS)-2-fluorotetrahydro-1H-pyrrolizin-7a(5H)-yl)methoxy)-4-(methyl((2S,3R)-2-methylpyrrolidin-3-yl)amino)-6-(trifluoromethyl)quinazolin-7-yl)benzo[b]thiophene-3-carbonitrile: A solution of tert-butyl (2S,3R)-3-[[7-[2-(tert-butoxycarbonylamino)-3-cyano-7-fluoro-benzothiophen-4-yl]-8-fluoro-2-[[(2R,8S)-2-fluoro-1,2,3,5,6,7-hexahydropyrrolizin-8-yl]methoxy]-6-(trifluoromethyl)quinazolin-4-yl]-methyl-amino]-2-methyl-pyrrolidine-1-carboxylate (1.00 eq. 200 mg, 0.228 mmol) and TFA (56.8 eq, 1.0 mL, 13.0 mmol) in DCM (4 mL) was stirred at 25° C. for 2 h. The reaction was monitored by LCMS. After the reaction was complete, the solvent was removed in vacuum. The mixture was then diluted with water and adjusted with NaHCO₃ until pH=8, and extracted with EA (3×20 mL). The combined extracts were washed with brine, dried over sodium sulfate, filtered and concentrated. The residue was purified by prep-TLC to give 2-amino-7-fluoro-4-[8-fluoro-2-[[(2R,8S)-2-fluoro-1,2,3,5,6,7-hexahydropyrrolizin-8-yl]methoxy]-4-[methyl-[(2S,3R)-2-methylpyrrolidin-3-yl]amino]-6-(trifluoromethyl)quinazolin-7-yl]benzothiophene-3-carbonitrile (200 mg, 0.207 mmol, 90.74% yield) as a yellow solid. LCMS ESI (+) m/z 676.1 (M+H).

Step G: Preparation of 4-(4-(((2S,3R)-1-acetyl-2-methylpyrrolidin-3-yl)(methyl)amino)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile: To a solution of 2-amino-7-fluoro-4-[8-fluoro-2-[[(2R,8S)-2-fluoro-1,2,3,5,6,7-hexahydropyrrolizin-8-yl]methoxy]-4-[methyl-[(2S,3R)-2-methylpyrrolidin-3-yl]amino]-6-(trifluoromethyl)quinazolin-7-yl]benzothiophene-3-carbonitrile (1.00 eq, 200 mg, 0.296 mmol) and TEA (10.0 eq, 300 mg, 2.96 mmol) in DCM (2 mL) was added acetic anhydride (1.00 eq, 0.028 mL, 0.296 mmol) at −40° C. under N₂. The mixture was stirred at −70° C. for 0.5 h. The mixture was then diluted with water and extracted with DCM (3×10 mL). The combined extracts were washed with brine, dried over sodium sulfate, filtered and concentrated, the residue was purified by prep-TLC to give 4-[4-[[(2S,3R)-1-acetyl-2-methyl-pyrrolidin-3-yl]-methyl-amino]-8-fluoro-2-[[(2R,8S)-2-fluoro-1,2,3,5,6,7-hexahydropyrrolizin-8-yl]methoxy]-6-(trifluoromethyl)quinazolin-7-yl]-2-amino-7-fluoro-benzothiophene-3-carbonitrile (33 mg, 0.0438 mmol, 14.79% yield) as an off-white solid. MS (ESI), m/z: calcd for C₃₄H₃₃F₆N₇O₂S, Exact Mass: 717.2, found RT=3.858 min, [M+H]+=718.3. ¹H NMR (400 MHz, CD₃OD) δ 8.29 (s, 1H), 7.21 (t, 1H), 7.00 (t, 1H), 5.38 (s, 0.5H), 5.24 (s, 0.5H), 5.05-5.02 (m, 1H), 4.38-4.24 (m, 3H), 3.99-3.87 (m, 1H), 3.75-3.88 (m, 1H), 3.48-3.36 (m, 3H), 3.27-3.23 (m, 2H), 3.04-3.00 (m, 1H), 2.59-2.54 (m, 1H), 2.38-2.11 (m, 7H), 2.01-1.90 (m, 3H), 1.48-1.37 (m, 3H).

### Synthetic Example 18: 2-amino-7-fluoro-4-[8-fluoro-2-[[(2R,8S)-2-fluoro-1,2,3,5,6,7-hexahydropyrrolizin-8-yl]methoxy]-6-(trifluoromethyl) quinazolin-7-yl]benzothiophene-3-carbonitrile (Compound 213)

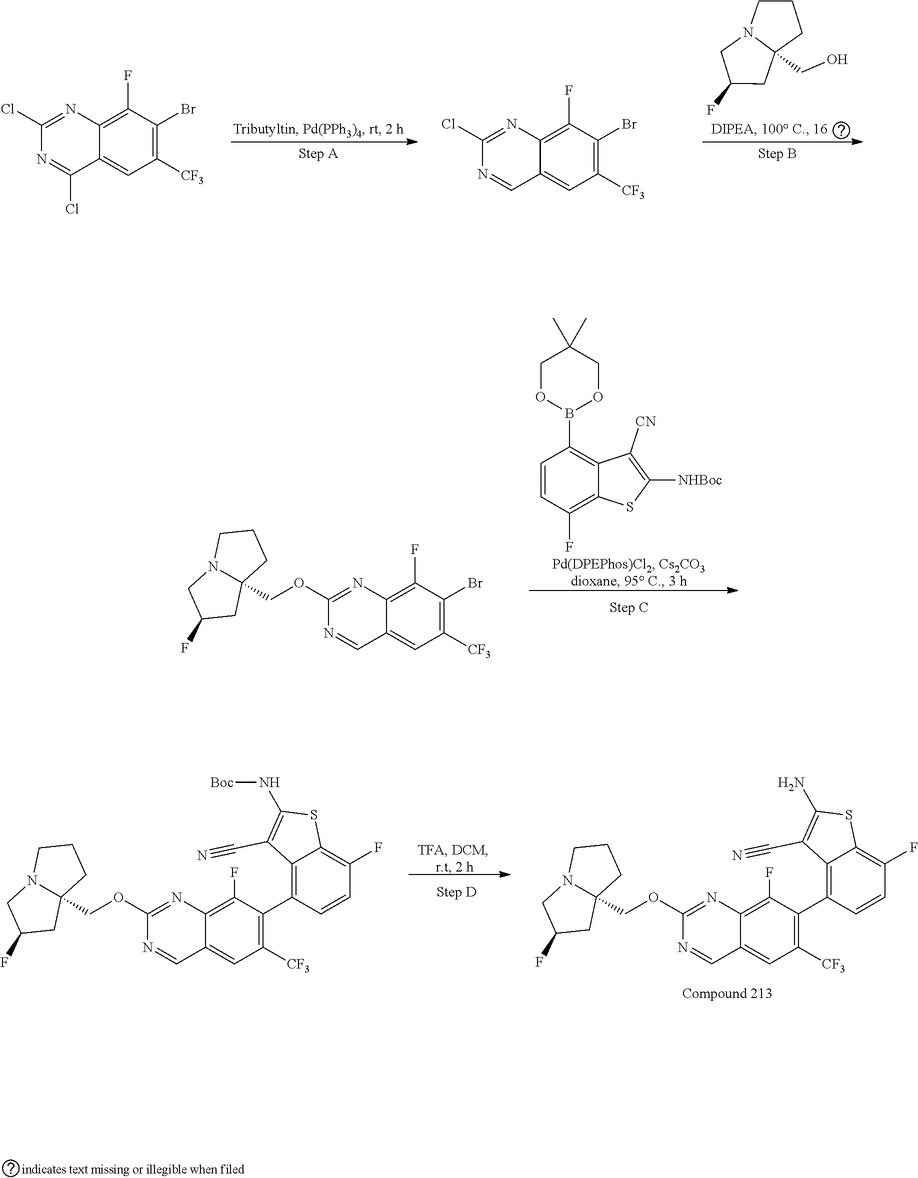

Step A: Preparation of 7-bromo-2-chloro-8-fluoro-6-(trifluoromethyl)quinazoline: To a solution of 7-bromo-2,4-dichloro-8-fluoro-6-(trifluoromethyl) quinazoline (1.00 eq, 500 mg, 1.37 mmol) in toluene (2 mL) was added bis(tributyltin) (1.00 eq, 0.69 mL, 1.37 mmol) and tetrakis(triphenylphosphine)palladium (O) (0.0500 eq, 79 mg, 0.0687 mmol) at 25° C. under N₂. After addition, the reaction mixture was stirred at 25° C. for 1 h. The mixture was diluted with water and extracted with EtOAc (4×10 mL). The combined organic phase was washed with brine, dried over sodium sulfate, filtered, and concentrated. The residue was purified by prep-TLC (PE/EA=5/1) to give 7-bromo-2-chloro-8-fluoro-6-(trifluoromethyl)quinazoline (370 mg, 0.999 mmol, 72.74% yield) as a light-yellow solid LCMS ESI (+) m/z 328.8 (M+H).

Step B: Preparation of 7-bromo-8-fluoro-2-(((2R,7aS)-2-fluorotetrahydro-1H-pyrrolizin-7a(5H)-yl)methoxy)-6-(trifluoromethyl)quinazoline: A mixture of 7-bromo-2-chloro-8-fluoro-6-(trifluoromethyl)quinazoline (1.00 eq, 370 mg, 1.12 mmol) and [(2R,8S)-2-fluoro-1,2,3,5,6,7-hexahydropyrrolizin-8-yl]methanol (1.00 eq, 179 mg, 1.12 mmol) in DIEA (30.0 eq, 6.0 mL, 33.7 mmol) was stirred at 100° C. for 16 h. The mixture was diluted with water and extracted with EtOAc (4×10 mL). The combined organic phase was washed with brine, dried over sodium sulfate, filtered, and concentrated. The residue was purified by prep-TLC (DCM/MeOH=20/1) to give 7-bromo-8-fluoro-2-[[(2R,8S)-2-fluoro-1,2,3,5,6,7-hexahydropyrrolizin-8-yl]methoxy]-6-(trifluoromethyl)quinazoline (120 mg, 0.191 mmol, 17.01% yield) as a yellow solid. LCMS ESI (+) m/z 451.9 (M+H).

Step C: Preparation of tert-butyl N-[3-cyano-7-fluoro-4-[8-fluoro-2-[[(2R,8S)-2-fluoro-1,2,3,5,6,7-hexahydropyrrolizin-8-yl]methoxy]-6-(trifluoromethyl)quinazolin-7-yl]benzothiophen-2-yl]carbamate: To a solution of 7-bromo-8-fluoro-2-[[(2R,8S)-2-fluoro-1,2,3,5,6,7-hexahydropyrrolizin-8-yl]methoxy]-6-(trifluoromethyl)quinazoline (1.00 eq, 120 mg, 0.265 mmol) in dioxane (10 mL) was added tert-butyl N-[3-cyano-4-(5,5-dimethyl-1,3,2-dioxaborinan-2-yl)-7-fluoro-benzothiophen-2-yl]carbamate (1.50 eq, 134 mg, 0.332 mmol), Cs₂CO₃ (3.00 eq, 0.36 mL, 0.663 mmol) and DPEPHOSPDCl₂ (0.400 eq, 63 mg, 0.0885 mmol). After addition, the reaction mixture was stirred at 95° C. for 3 h under argon. The mixture was diluted with water and extracted with EtOAc (4×10 mL). The combined organic phase was washed with brine, dried over sodium sulfate, filtered, and concentrated. The residue was purified by prep-TLC (DCM/MeOH=20/1) to give tert-butyl N-[3-cyano-7-fluoro-4-[8-fluoro-2-[[(2R,8S)-2-fluoro-1,2,3,5,6,7-hexahydropyrrolizin-8-yl]methoxy]-6-(trifluoromethyl)quinazolin-7-yl]benzothiophen-2-yl]carbamate (40 mg, 0.0338 mmol, 15.26% yield) as a yellow solid. LCMS ESI (+) m/z 664.0 (M+H).

Step D: Preparation of 2-amino-7-fluoro-4-[8-fluoro-2-[[(2R,8S)-2-fluoro-1,2,3,5,6,7-hexahydropyrrolizin-8-yl]methoxy]-6-(trifluoromethyl)quinazolin-7-yl]benzothiophene-3-carbonitrile: To a solution of tert-butyl N-[3-cyano-7-fluoro-4-[8-fluoro-2-[[(2R,8S)-2-fluoro-1,2,3,5,6,7-hexahydropyrrolizin-8-yl]methoxy]-6-(trifluoromethyl)quinazolin-7-yl]benzothiophen-2-yl]carbamate (1.00 eq, 40 mg, 0.0603 mmol) in DCM (1.5 mL) was added TFA (123 eq, 0.50 mL, 6.49 mmol) at 25° C. After addition, the reaction mixture was stirred at 25° C. for 2 h. The reaction solution was adjusted to pH 8 with saturated sodium bicarbonate aqueous solution, extracted with EtOAc (4×10 mL). The combined organic phase was washed with brine, dried over sodium sulfate, and concentrated. The residue was purified by prep-TLC (DCM/MeOH=10/1) to give 2-amino-7-fluoro-4-[8-fluoro-2-[[(2R,8S)-2-fluoro-1,2,3,5,6,7-hexahydropyrrolizin-8-yl]methoxy]-6-(trifluoromethyl)quinazolin-7-yl]benzothiophene-3-carbonitrile (11 mg, 0.0190 mmol, 36.00% yield) as a yellow solid. LCMS ESI (+) m/z 567.0 (M+H). ¹H NMR (400 MHz, CD₃OD) δ 9.60 (s, 1H), 8.41 (s, 1H), 7.21-7.25 (m, 1H), 7.02 (t, 1H), 5.39 (s, 0.511), 5.25 (s, 0.511), 4.35-4.45 (m, 2H), 3.22-3.27 (m, 3H), 3.00-3.07 (m, 1H), 2.16-2.42 (m, 4H), 1.99-2.06 (m, 2H).

### Synthetic Example 19: 4-((7S)-4-(6-oxa-3-azabicyclo[3.1.1]heptan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 275) and 4-((7R)-4-(6-oxa-3-azabicyclo[3.1.1]heptan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 276)

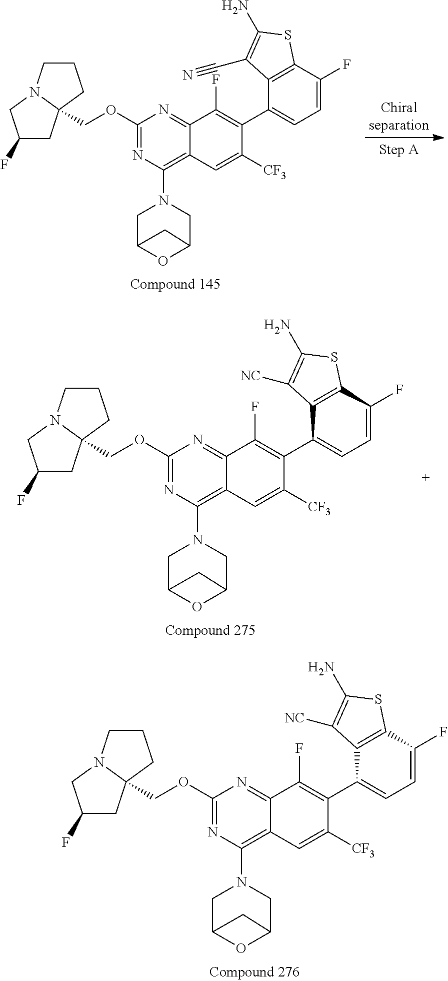

Step A: Preparation of 4-((7S)-4-(6-oxa-3-azabicyclo[3.1.1]heptan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 275) and 4-((7R)-4-(6-oxa-3-azabicyclo[3.1.1]heptan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 276): 2-amino-7-fluoro-4-[8-fluoro-2-[[(2S,8R)-2-fluoro-1,2,3,5,6,7-hexahydropyrrolizin-8-yl]methoxy]-4-(6-oxa-3-azabicyclo[3.1.1]heptan-3-yl)-6-(trifluoromethyl)quinazolin-7-yl]benzothiophene-3-carbonitrile (Compound 145, 92.6 mg, 0.138 mmol) was separated with chiral SFC (CHIRAL ART Cellulose-SC 3 cm×25 cm, 5 μm, CO₂:MeOH(0.2% 2 mM NH₃-MeOH)=55:45) to give one atropisomer, 4-((7S)-4-(6-oxa-3-azabicyclo[3.1.1]heptan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 275) (28 mg, 0.042 mmol), as first peak; LCMS m/z [M+1]: 661.3. ¹H NMR (400 MHz, CD₃OD) δ 8.59 (s, 1H), 7.16-7.24 (m, 1H), 7.01 (t, J=9.2 Hz, 1H), 5.22-5.37 (m, 1H), 4.46-4.59 (m, 3H), 4.21-4.39 (m, 4H), 3.12-3.26 (m, 4H), 2.96-3.04 (m, 1H), 2.09-2.40 (m, 4H), 1.84-2.03 (m, 4H); and give the other atropisomer, 4-((7R)-4-(6-oxa-3-azabicyclo[3.1.1]heptan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 276): (27.8 mg, 0.042 mmol), as second peak. LCMS m/z [M+H: 661.3. ¹H NMR (400 MHz, CD₃OD) δ 8.59 (s, 1H), 7.16-7.24 (m, 1H), 7.01 (t, J=9.2 Hz, 1H), 5.21-5.38 (m, 1H), 4.44-4.64 (m, 3H), 4.18-4.42 (m, 4H), 3.10-3.29 (m, 4H), 2.93-3.03 (m, 1H), 1.81-2.40 (m, 8H).

### Synthetic Example 20: 2-amino-7-fluoro-4-[8-fluoro-2-[[(2R,8S)-2-fluoro-1,2,3,5,6,7-hexahydropyrrolizin-8-yl]methoxy]-4-[(1R,4R)-2-oxa-5-azabicyclo[2.2.1]heptan-5-yl]-6-(trifluoromethyl)quinazolin-7-yl]benzothiophene-3-carbonitrile (Compound 253), 4-((S)-4-((1R,4R)-2-oxa-5-azabicyclo[2.2.1]heptan-5-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 287), and 4-((R)-4-((1R,4R)-2-oxa-5-azabicyclo[2.2.1]heptan-5-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 288)

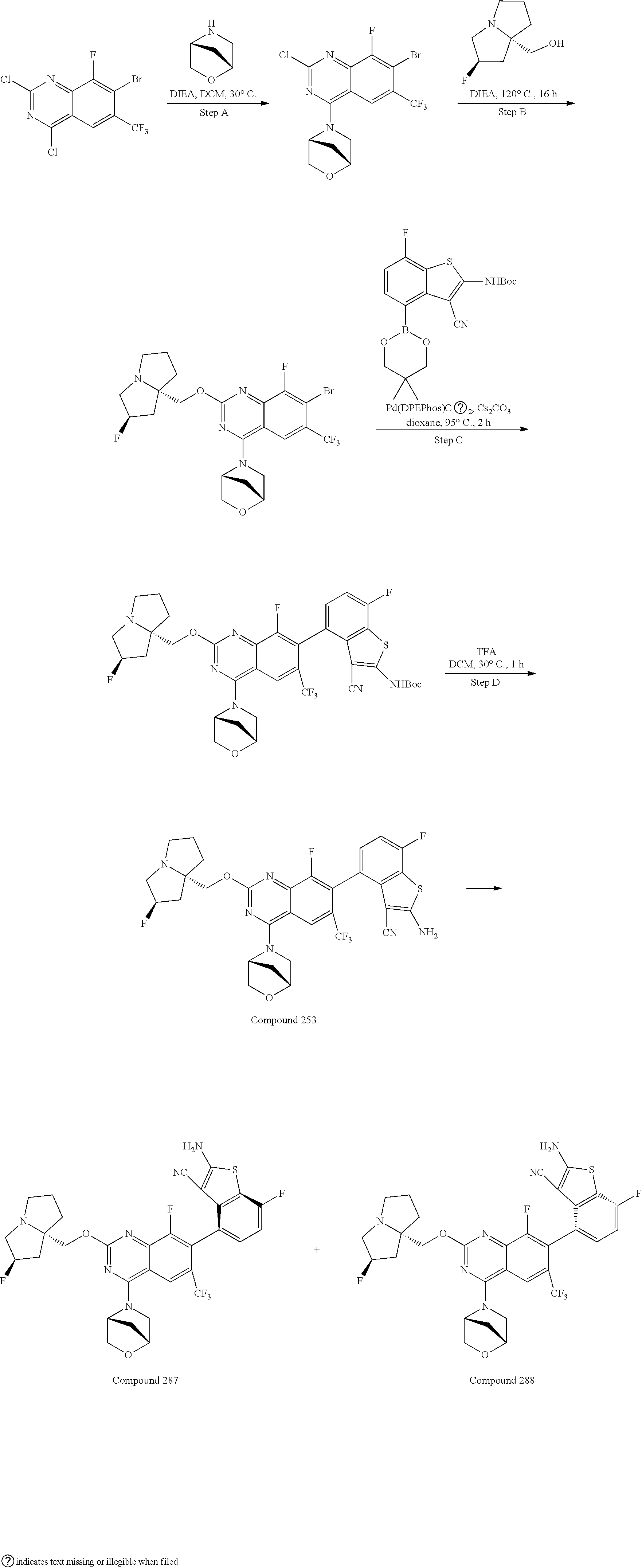

Step A: Preparation of (1R,4R)-5-[7-bromo-2-chloro-8-fluoro-6-(trifluoromethyl)quinazolin-4-yl]-2-oxa-5-azabicyclo[2.2.1]heptane: To a mixture of 7-bromo-2,4-dichloro-8-fluoro-6-(trifluoromethyl)quinazoline (1.00 eq. 400 mg, 1.10 mmol) in DCM (5 mL) were added DIEA (4.00 eq. 0.78 mL, 4.40 mmol) and (1R,4R)-2-oxa-5-azabicyclo[2.2.1]heptane (1.20 eq, 131 mgs, 1.32 mmol) in DCM at 0° C. The mixture was stirred at 30° C. for 2 hrs. LCMS trace indicated the desired product was formed. The reaction was concentrated to dryness and the residue was taken up in EtOAc (30 mL). The organic layers were washed with 2×30 mL water then 1×30 mL saturated brine solution, the organic layers were separated and dried (MgSO₄) before concentration to dryness. The crude was purified by Prep-TLC(EtOAc/PE=1:4) to give (1R,4R)-5-[7-bromo-2-chloro-8-fluoro-6-(trifluoromethyl)quinazolin-4-yl]-2-oxa-5-azabicyclo[2.2.1]heptane (360 mg, 0.844 mmol, 76.78% yield). LCMS ESI(+) m/z 425.8 (M+H).

Step B: Preparation of (1R,4R)-5-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-2-oxa-5-azabicyclo[2.2.1]heptane: (1R,4R)-5-[7-bromo-2-chloro-8-fluon-6-(trifluoromethyl)quinazolin-4-yl]-2-oxa-5-azabicyclo[2.2.1]heptane (1.00 eq, 360 mg, 0.844 mmol) was dissolved in DIEA (55.7 eq, 8.4 mL, 47.0 mmol), and to this was added [(2R,8S)-2-fluoro-1,2,3,5,6,7-hexahydropyrrolizin-8-yl]methanol (4.00 eq, 537 mg, 3.38 mmol), the mixture was stirred 16 hrs at 120° C. LCMS trace indicated the desired product was formed. The reaction was concentrated to dryness and the residue was taken up in EtOAc (30 mL). The organic layers were washed with 2×30 mL water, then 1×30 mL saturated brine solution, the organic layers were separated and dried (MgSO₄) before concentration to dryness, the crude was then purified by Prep-TLC(EtOAc/PE=1:1) to give (1R,4R)-5-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-2-oxa-5-azabicyclo[2.2.1]heptane (250 mg, 0.455 mmol, 53.93% yield). LCMS ESI (+) m/z 549.0 (M+H).

Step C: Preparation of tert-butyl (4-(4-((1R,4R)-2-oxa-5-azabicyclo[2.2.1]heptan-5-yl)-8-fluoro-2-(((2R,7aS)-2-fluorotetrahydro-1H-pyrrolizin-7a(5H)-yl)methoxy)-6-(trifluoromethyl)quinazolin-7-yl)-3-cyano-7-fluorobenzo[b]thiophen-2-yl)carbamate: To the solution of (1R,4R)-5-[7-bromo-8-fluoro-2-[[(2R,8S)-2-fluoro-1,2,3,5,6,7-hexahydropyrrolizin-8-yl]methoxy]-6-(trifluoromethyl)quinazolin-4-yl]-2-oxa-5-azabicyclo[2.2.1]heptane (1.00 eq, 250 mg, 0.455 mmol) in 1,4-dioxane (4 mL) were added Cs₂CO₁ (3.00 eq, 445 mg, 1.37 mmol), tert-butyl N-[3-cyano-4-(5,5-dimethyl-1,3,2-dioxaborinan-2-yl)-7-fluoro-benzothiophen-2-yl]carbamate (3.00 eq, 552 mg, 1.37 mmol) and Pd(DPEPhos)Cl₂ (0.400 eq, 130 mg, 0.182 mmol). The mixture was bubbled with Ar for 1-2 min and then sealed, the mixture was stirred for 2 hrs at 95° C. The mixture was extracted with EtOAc (20 mL×3) and the combined organic layers were concentrated to dryness under vacuum, the residue was purified by Prep-TLC to give tert-butyl (4-(4-((1R,4R)-2-oxa-5-azabicyclo[2.2.1]heptan-5-yl)-8-fluoro-2-(((2R,7aS)-2-fluorotetrahydro-1H-pyrrolizin-7a(5H)-yl)methoxy)-6-(trifluoromethyl)quinazolin-7-yl)-3-cyano-7-fluorobenzo[b]thiophen-2-yl)carbamate (120 mg, 0.158 mmol, 34.66% yield). LCMS ESI (+) m/z 761.2 (M+H).

Step D: Preparation of 2-amino-7-fluoro-4-[8-fluoro-2-[[(2R,8S)-2-fluoro-1,2,3,5,6,7-hexahydropyrrolizin-8-yl]methoxy]-4-[(1R,4R)-2-oxa-5-azabicyclo[2.2.1]heptan-5-yl]-6-(trifluoromethyl)quinazolin-7-yl]benzothiophene-3-carbonitrile (Compound 253), 4-((S)-4-((1R,4R)-2-oxa-5-azabicyclo[2.2.1]heptan-5-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 287), and 4-((R)-4-((1R,4R)-2-oxa-5-azabicyclo[2.2.1]heptan-5-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 288): To the solution of tert-butyl N-[3-cyano-7-fluor-4-[8-fluoro-2-[[(2S,8R)-2-fluoro-1,2,3,5,6,7-hexahydropyrrolizin-8-yl]methoxy]-4-[(1R,4R)-2-oxa-5-azabicyclo[2.2.1]heptan-5-yl]-6-(trifluoromethyl)quinazolin-7-yl]benzothiophen-2-yl]carbamate (1.00 eq, 120 mg, 0.158 mmol) in DCM (2 mL) was added TFA (1.0 mL, 13.0 mmol), the mixture was stirred for 2 hrs at 30° C. The organic layers were concentrated to dryness under vacuum, the residue was purified by Prep-HPLC to give 2-amino-7-fluoro-4-[8-fluoro-2-[[(2R,8S)-2-fluoro-1,2,3,5,6,7-hexahydropyrrolizin-8-yl]methoxy]-4-[(1R,4R)-2-oxa-5-azabicyclo[2.2.1]heptan-5-yl]-6-(trifluoromethyl)quinazolin-7-yl]benzothiophene-3-carbonitrile (Compound 253, 75 mg, 0.113 mmol, 71.51% yield). LCMS ESI (+) m/z 661.2 (M+H). ¹H NMR (400 MHz, CD₃OD) δ 8.35 (s, 1H), 7.19-7.24 (m, 1H), 7.02 (t, J=9.3 Hz, 1H), 5.48-5.61 (m, 2H), 4.56-4.76 (m, 2H), 4.33 (d, J=9.9 Hz, 1H), 3.80-4.12 (m, 6H), 3.41-3.49 (m, 1H), 2.05-2.79 (m, 9H), 2-amino-7-fluoro-4-[8-fluoro-2-[[(2R,8S)-2-fluoro-1,2,3,5,6,7-hexahydropyrrolizin-8-yl]methoxy]-4-[(1R,4R)-2-oxa-5-azabicyclo[2.2.1]heptan-5-yl]-6-(trifluoromethyl)quinazolin-7-yl]benzothiophene-3-carbonitrile (Compound 253) was separated with chiral SFC (CHIRALPAK-IH 3 cm×25 cm, 5 μm, CO₂:MeOH(0.2% 2 mM NH₃-MeOH)=65:35) to give one atropisomer, 4-((S)-4-((1R,4R)-2-oxa-5-azabicyclo[2.2.1]heptan-5-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 287) (23 mg, 0.0356 mmol, 31.45% yield), as first peak; LCMS ESI (+) m/z 661.2 (M+H). ¹H NMR (400 MHz, Methanol-d4) δ 8.30 (s, 1H), 7.20-7.24 (m, 1H), 7.01 (d, J=9.6 Hz, 1H), 5.53 (s, 1H), 5.41 (t, J=54 Hz, 1H), 4.21-4.35 (m, 3H), 3.98-4.10 (m, 3H), 3.31-3.38 (m, 1H), 3.21-3.27 (m, 3H), 3.01-3.07 (m, 1H), 1.88-2.37 (m, 8H); and give 4-((R)-4-((1R,4R)-2-oxa-5-azabicyclo[2.2.1]heptan-5-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile (Compound 288) (26 mg, 0.0400 mmol, 35.38% yield) as the second peak. LCMS ESI (+) m/z 661.2 (M+H). ¹H NMR (400 MHz, Methanol-d4) δ 8.30 (s, 1H), 7.20-7.24 (m, 1H), 7.01 (d, J=9.3 Hz, 1H), 5.50 (s, 1H), 5.41 (t, J=53.7 Hz, 1H), 4.27-4.39 (m, 3H), 3.99-4.11 (m, 3H), 3.31-3.38 (m, 1H), 3.21-3.27 (m, 3H), 3.01-3.07 (m, 1H), 1.88-2.37 (m, 8H).

### Synthetic Example 21: 2-amino-7-fluoro-4-[8-fluoro-2-[[(2R,8S)-2-fluoro-1,2,3,5,6,7-hexahydropyrrolizin-8-yl]methoxy]-4-methyl-6-(trifluoromethyl)quinazolin-7-yl]benzothiophene-3-carbonitrile (Compound 318)

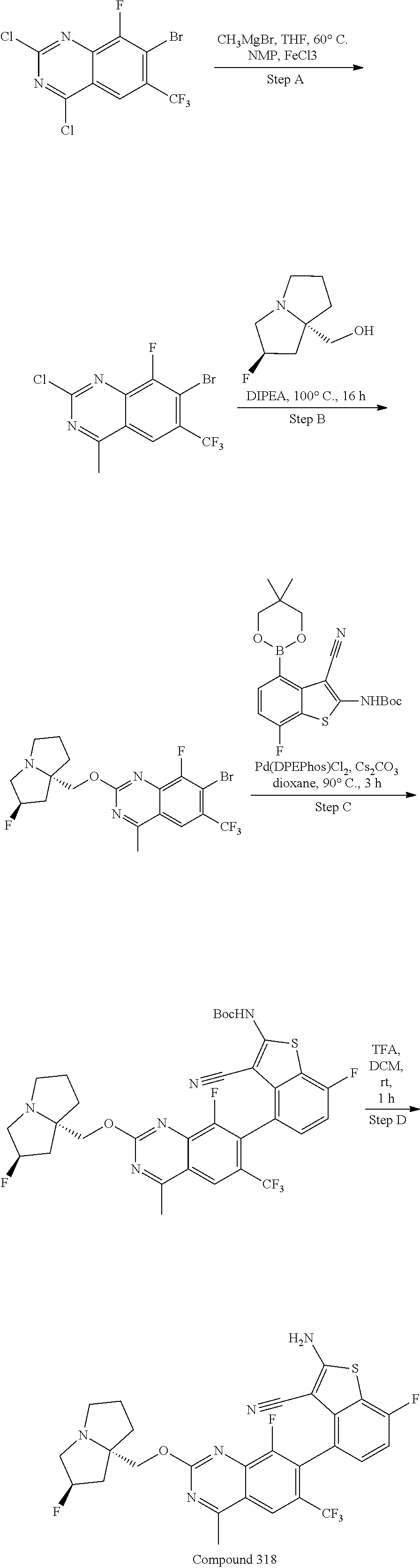

Step A: Preparation of 7-bromo-2-chloro-8-fluoro-4-methyl-6-(trifluoromethyl)quinazoline: To a solution of 7-bromo-2,4-dichloro-8-fluoro-6-(trifluoromethyl)quinazoline (1.00 eq, 600 mg, 1.65 mmol) in THF (12 mL) was added NMP (1.50 eq, 245 mg, 2.47 mmol), FeCl₃ (0.100 eq, 27 mg, 0.165 mmol) and 3.0 M methyl magnesium bromide (1.50 eq, 0.82 mL, 2.47 mmol) at 0° C. After addition the reaction was stirred at 60° C. for 4 h under N₂. The reaction was added water and extracted with EA for 3 times, the organic phase was washed with brine, dry and concentrate under reduced pressure to obtain the crude product, the crude product was purified by prep-TLC to give 7-bromo-2-chloro-8-fluoro-4-methyl-6-(trifluoromethyl)quinazoline (80 mg, 0.116 mmol, 7.06% yield). LCMS ESI (+) m/z 343 (M+H).

Step B: Preparation of 7-bromo-8-fluoro-2-[[(2R,8S)-2-fluoro-1,2,3,5,6,7-hexahydropyrrolizin-8-yl]methoxy]-4-methyl-6-(trifluoromethyl)quinazoline: To a solution of 7-bromo-2-chloro-8-fluoro-4-methyl-6-(trifluoromethyl)quinazoline (1.00 eq, 80 mg, 0.233 mmol) in DIPEA (48.2 eq, 2.0 mL, 11.2 mmol) was added [(2R,8S)-2-fluoro-1,2,3,5,6,7-hexahydropyrrolizin-8-yl]methanol (2.00 eq, 74 mg, 0.466 mmol). After addition, the reaction was stirred at 100° C. for 16 h. To the reaction mixture was added water and extracted with EA for 3 times, the organic phase was washed with brine, dried and concentrated under reduced pressure to obtain the crude product, the crude product was purified by prep-TLC to give 7-bromo-8-fluoro-2-1(2R,8S)-2-fluoro-1,2,3,5,6,7-hexahydropyrrolizin-8-yl]methoxy]-4-methyl-6-(trifluoromethyl)quinazoline (60 mg, 0.0772 mmol, 33.15% yield). LCMS ESI (+) m/z 466 (M+H).

Step C: Preparation of tert-butyl N-[3-cyano-7-fluoro-4-[8-fluoro-2-[[(2R,8S)-2-fluoro-1,2,3,5,6,7-hexahydropyrrolizin-8-yl]methoxy]-4-methyl-6-(trifluoromethyl)quinazolin-7-yl]benzothiophen-2-yl]carbamate: To a solution of 7-bromo-8-fluoro-2-[[(2R,8S)-2-fluoro-1,2,3,5,6,7-hexahydropyrrolizin-8-yl]methoxy]-4-methyl-6-(trifluoromethyl)quinazoline (1.00 eq, 50 mg, 0.107 mmol) in 1,4-dioxane (3 mL) was added tert-butyl N-[3-cyano-4-(5,5-dimethyl-1,3,2-dioxaborinan-2-yl)-7-fluoro-benzothiophen-2-yl]carbamate (1.50 eq, 65 mg, 0.161 mmol), Cs₂CO, (3.00 eq, 105 mg, 0.322 mmol) and Pd(DPEPhos)Cl₂ (0.40 eq, 31 mg, 0.0429 mmol). After addition the reaction was stirred at 90° C. for 3 h under N₂. To the reaction mixture was added water and extracted with EA for 3 times, the organic phase was washed with brine, dried and concentrated under reduced pressure to obtain the crude product, the crude product was purified by prep-TLC to give tert-butyl N-[3-cyano-7-fluoro-4-[8-fluoro-2-[[(2R,8S)-2-fluoro-1,2,3,5,6,7-hexahydropyrrolizin-8-yl]methoxy]-4-methyl-6-(trifluoromethyl)quinazolin-7-yl]benzothiophen-2-yl]carbamate (40 mg, 0.0295 mmol, 27.52% yield). LCMS ESI (+) m/z 678 (M+H).

Step D: Preparation of 2-amino-7-fluoro-4-[8-fluoro-2-[[(2R,8S)-2-fluoro-1,2,3,5,6,7-hexahydropyrrolizin-8-yl]methoxy]-4-methyl-6-(trifluoromethyl)quinazolin-7-yl]benzothiophene-3-carbonitrile: To a solution of tert-butyl N-[3-cyano-7-fluoro-4-[8-fluoro-2-[[(2R,8S)-2-fluoro-1,2,3,5,6,7-hexahydropyrrolizin-8-yl]methoxy]-4-methyl-6-(trifluoromethyl)quinazolin-7-yl]benzothiophen-2-yl]carbamate (1.00 eq, 40 mg, 0.0236 mmol) in DCM (2 mL) was added TFA (0.50 mL). After addition, the reaction was stirred at 25° C. for 2 h. To the mixture was added EA and saturated K₂CO₃ until pH=9 and stirred for 1 h. The solution was extracted with EA 3 times, the organic phase was washed with brine, dried and concentrated under reduced pressure to obtain the crude product. The crude product was purified by prep-TLC to give 2-amino-7-fluoro-4-[8-fluoro-2-[[(2R,8S)-2-fluoro-1,2,3,5,6,7-hexahydropyrrolizin-8-yl]methoxy]-4-methyl-6-(trifluoromethyl)quinazolin-7-yl]benzothiophene-3-carbonitrile (6.1 mg, 0.0102 mmol, 43.08% yield). LCMS ESI (+) m/z 578 (M+H). ¹HNMR (400 MHz, CD₃OD) δ 8.42 (s, 1H), 7.20-7.25 (m, 1H), 7.02 (t, 1H), 5.41 (s, 0.5H), 5.28 (s, 0.5H), 4.35-4.47 (m, 2H), 3.32-3.45 (m, 2H), 3.22-3.27 (m, 1H), 3.05-3.12 (m, 1H), 3.02 (s, 3H), 2.17-2.42 (m, 3H), 1.92-2.08 (m, 3H).

### Synthetic Example 22: 2-amino-4-[4-[ethyl-[(3S)-1-(2-methylpropanoyl)pyrrolidin-3-yl]amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (Compound 469)

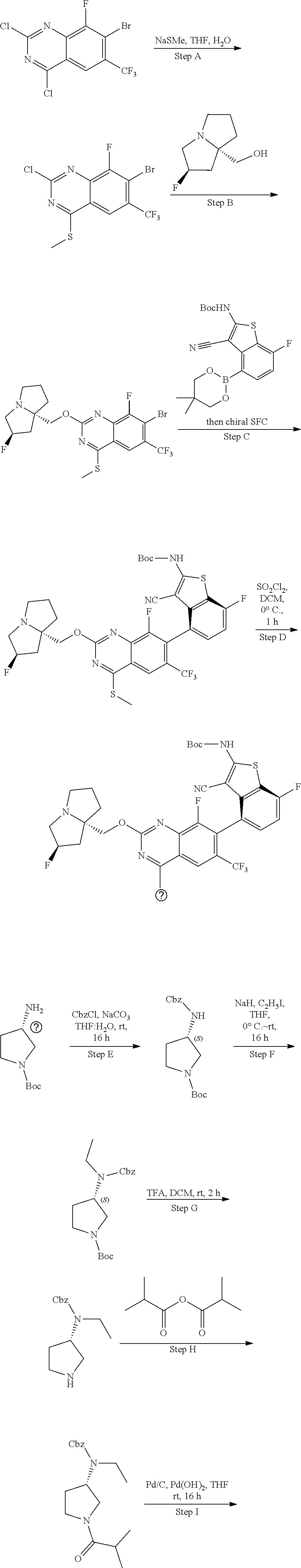

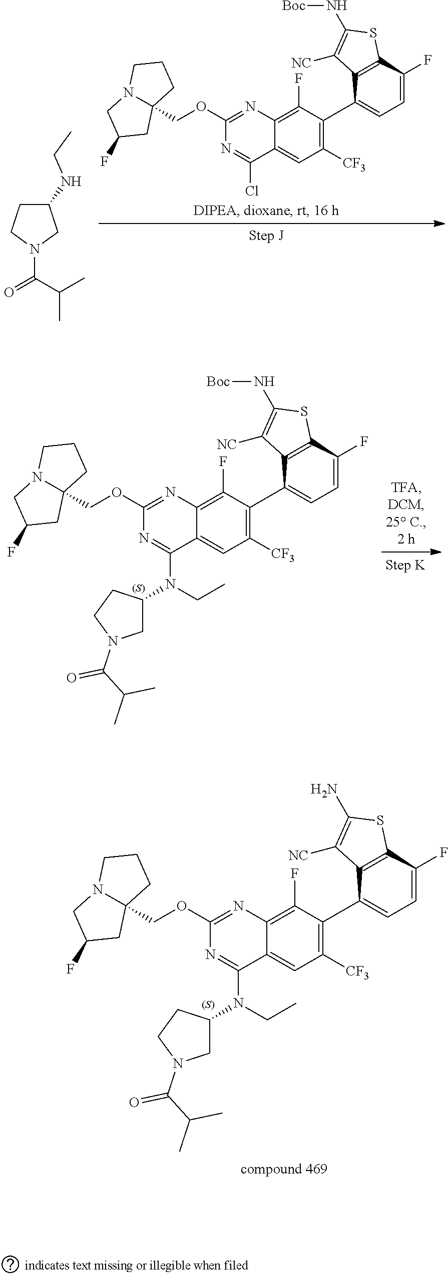

Step A: Preparation of 7-bromo-2-chloro-8-fluoro-4-(methylthio)-6-(trifluoromethyl)quinazoline: To a solution of 7-bromo-2,4-dichloro-8-fluoro-6-(trifluoromethyl)quinazoline (1.00 eq, 100 g, 0.27 mol) in THF (1500 mL) was added sodium thiomethoxide (2.20 eq, 41.6 g, 0.59 mol, 20% in water) with dropwise at 0° C. under nitrogen atmosphere. The resulting mixture was stirred at 0° C. for 1 h under nitrogen atmosphere. The reaction was monitored by LCMS. The resulting mixture was diluted with water and extracted with DCM (2 L×2). The combined organic phase was washed with brine, dried over sodium sulfate, filtered, and concentrated under vacuum. The residue was triturated with petroleum (500 mL) and the precipitated solids were collected by filtration to afford 7-bromo-2-chloro-8-fluoro-4-(methylthio)-6-(trifluoromethyl)quinazoline (90 g, 0.24 mol, 88.89% yield) as alight yellow solid. LCMS ESI (+) m/z 374.8, 376.8 (M+H).

Step B: Preparation of (3-cyano-7-fluoro-4-(8-fluoro-2-(((2R,7aS)-2-fluorotetrahydro-1H-pyrrolizin-7a(5H)-yl)methoxy)-4-(methylthio)-6-(trifluoromethyl)quinazolin-7-yl)benzo[b]thiophen-2-yl)carbamate: To a solution of 7-bromo-2-chloro-8-fluoro-4-(methylthio)-6-(trifluoromethyl)quinazoline (1.00 eq, 85 g, 0.23 mol) in 1,4-dioxane (850 mL) was added DIEA (5.00 eq, 148 g, 1.15 mol) and ((2R,7aS)-2-fluorotetrahydro-1H-pyrrolizin-7a(5H)-yl)methanol (1.30 eq, 47.5 g, 0.299 mmol) at 25° C. under nitrogen atmosphere. The resulting mixture was stirred at 90° C. for 16 h under nitrogen atmosphere. The reaction was monitored by LCMS. The resulting mixture was concentrated under vacuum, diluted with water and extracted with ethyl acetate (1 L×3). The combined organic phase was washed with brine, dried over sodium sulfate, filtered and concentrated under vacuum. The residue was purified by silica gel column chromatography eluting with MeOH in DCM from 1% to 4% to afford 7-bromo-8-fluoro-2-(((2R,7aS)-2-fluorotetrahydro-1H-pyrrolizin-7a(5H)-yl)methoxy)-4-(methylthio)-6-(trifluoromethyl)quinazoline (85.2 g, 0.17 mol, 73.9% yield) as a yellow solid. LCMS ESI (+) m/z 497.9, 499.9 (M+H).

Step C: Preparation of tert-butyl (3-cyano-7-fluoro-4-((S)-8-fluoro-2-(((2R,7aS)-2-fluorotetrahydro-1H-pyrrolizin-7a(5H)-yl)methoxy)-4-(methylthio)-6-(trifluoromethyl)quinazolin-7-yl)benzo[b]thiophen-2-yl)carbamate: To a solution of 7-bromo-8-fluoro-2-(((2R,7aS)-2-fluorotetrahydro-IH-pyrrolizin-7a(5H)-yl)methoxy)-4-(methylthio)-6-(trifluoromethyl)quinazoline (1.00 eq, 54 g, 0.11 mol)) in 1,4-dioxane (2160 mL) were added tert-butyl (3-cyano-4-(5,5-dimethyl-1,3,2-dioxaborinan-2-yl)-7-fluorobenzo[b]thiophen-2-yl)carbamate (1.50 eq, 68.7 g, 0.17 mol), Cs₂CO₃ (3.00 eq. 107 g, 0.33 mol) and DPEPhosPdCl₂ (0.20 eq, 15.7 g, 0.02 mmol) at 25° C. under nitrogen atmosphere. The resulting mixture was stirred at 90° C. for 16 h under nitrogen atmosphere. The reaction was monitored by LCMS. The resulting mixture was diluted with water and extracted with ethyl acetate (600 mL×4). The combined organic phase was washed with brine, dried over sodium sulfate, filtered and concentrated under vacuum. The residue was purified by silica gel column chromatography eluting with ethyl acetate in petroleum from 0% to 100% to give product. The racemate (30 g) was separated by chiral SFC (Daicel IC 2 cm×25 cm, 5 μm, CO₂:EtOH=80:20 (0.1% DEA in EtOH)) to afford tert-butyl (3-cyano-7-fluoro-4-((S)-8-fluoro-2-(((2R,7aS)-2-fluorotetrahydro-1H-pyrrolizin-7a(5H)-yl)methoxy)-4-(methylthio)-6-(trifluoromethyl)quinazolin-7-yl)benzo[b]thiophen-2-yl)carbamate (11 g, 15.5 mmol, 14.3% yield) as the first peak and yellow solid. LCMS ESI (+) m/z 710.0 (M+H).

Step D: Preparation of tert-butyl N-[4-[4-chloro-8-fluoro-2-[[(2R,8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate: To a solution of tert-butyl N-[3-cyano-7-fluoro-4-[8-fluoro-2-[[(2R,8S)-2-fluoro-1,2,3,5,6,7-hexahydropyrrolizin-8-yl]methoxy]-4-methylsulfanyl-6-(trifluoromethyl)quinazolin-7-yl]benzothiophen-2-yl]carbamate (1.00 eq, 5.00 g, 7.05 mmol) in DCM (200 mL) was added dropwise sulfuryl chloride (5.00 eq, 4.75 g, 35.20 mmol) at 0° C. under nitrogen atmosphere. The resulting mixture was stirred at 0° C. for 1 h under nitrogen atmosphere. The reaction was monitored by LCMS. The resulting mixture was adjusted to pH=8 with saturated sodium bicarbonate aqueous solution and extracted with DCM (200 mL×3). The combined organic phase was washed with water and saturated brine solution, dried over sodium sulfate, filtered and concentrated under vacuum to afford the crude tert-butyl N-[4-[4-chloro-8-fluoro-2-[[(2R,8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate (4.5 g, 6.44 mmol, 91.83% yield) as a yellow solid, which was used in next step directly without further purification. LCMS ESI (+) m/z 698.1 (M+H).

Step E: Preparation of tert-butyl (3S)-3-(benzyloxycarbonylamino)pyrrolidine-1-carboxylate: To a solution of tert-butyl (3S)-3-aminopyrrolidine-1-carboxylate (1.00 eq, 10.00 g, 53.7 mmol) in THF (100 mL) and water (100 mL) was added NaHCO₃ (2.00 eq, 9.02 g, 107.4 mmol). Then benzil chloroformate (1.20 eq, 10.99 g, 64.4 mmol) was added to above mixture dropwise at 0° C. under nitrogen atmosphere. The resulting mixture was stirred at 25° C. for 16 h under nitrogen atmosphere. The reaction was monitored by LCMS. The resulting mixture was diluted with water and extracted with ethyl acetate (100 mL×3). The combined organic phase was washed with brine, dried over sodium sulfate, filtered and concentrated under vacuum. The residue was purified by silica gel column chromatography eluting with ethyl acetate in petroleum from 0% to 25% to afford tert-butyl (3S)-3-(benzyloxycarbonylamino)pyrrolidine-1-carboxylate (13.44 g, 41.97 mmol, 78.16% yield) as a yellow oil. LCMS ESI (+) m/z 265.0 (M+H−56).

Step F: Preparation of tert-butyl (3S)-3-[benzyloxycarbonyl(ethyl)amino]pyrrolidine-1-carboxylate: To DMF (140 mL) was added NaH (1.50 eq, 1.51 g, 62.96 mmol) at 0° C. under nitrogen atmosphere. Then a solution of tert-butyl (3S)-3-(benzyloxycarbonylamino)pyrrolidine-1-carboxylate (1.00 eq, 13.44 g, 41.97 mmol) in DMF (100 mL) was added to above mixture dropwise at 0° C. under nitrogen atmosphere. The resulting mixture was stirred at 0° C. for 1 h under nitrogen atmosphere. Then CH₃CH₂I (1.20 eq, 7.86 g, 50.36 mmol) was added to above mixture dropwise at 0° C. under nitrogen atmosphere. The resulting mixture was stirred at 25° C. for 16 h under nitrogen atmosphere. The reaction was monitored by LCMS. The mixture was diluted with water and extracted with ethyl acetate (200 mL×3). The combined organic phase was washed with brine, dried over sodium sulfate, filtered and concentrated under vacuum. The residue was purified by silica gel column chromatography eluting with ethyl acetate in petroleum from 0% to 30% to afford tert-butyl (3S)-3-[benzyloxycarbonyl(ethyl)amino]pyrrolidine-1-carboxylate (10.02 g, 28.79 mmol, 68.60% yield) as a yellow oil. LCMS ESI (+) m/z 249.1 (M+H−100).

Step G: Preparation of benzyl N-ethyl-N-[(3S)-pyrrolidin-3-yl]carbamate: To a solution of tert-butyl (3S)-3-[benzyloxycarbonyl(ethyl)amino]pyrrolidine-1-carboxylate (1.00 eq, 10.02 g, 28.79 mmol) in DCM (80 mL) was added TFA (9.03 eq, 20 mL, 260 mmol) at 0° C. under nitrogen atmosphere. The resulting mixture was stirred at 25° C. for 2 h under nitrogen atmosphere. The reaction was monitored by LCMS. The resulting mixture was concentrated under vacuum to afford crude of benzyl N-ethyl-N-[(3S)-pyrrolidin-3-yl]carbamate (12.55 g, 28.79 mmol, 100.00% yield) as a brown oil, which was used in next step directly without further purification. LCMS ESI (+) m/z 249.1 (M+H).

Step H: Preparation of benzyl N-ethyl-N-[(3S)-1-(2-methylpropanoyl)pyrrolidin-3-yl]carbamate: To a solution of benzyl N-ethyl-N-[(3S)-pyrrolidin-3-yl]carbamate (1.00 eq, 12.55 g, 28.79 mmol) in DCM (120 mL) was added DIPEA (3.00 eq, 11.23 g, 86.37 mmol). Then isobutyric anhydride (1.50 eq, 6.82 g, 43.19 mmol) was added to above mixture dropwise at −40° C. under nitrogen atmosphere. The resulting mixture was stirred at 25° C. for 16 h under nitrogen atmosphere. The reaction was monitored by LCMS.

The mixture was diluted with water and extracted with ethyl acetate (150 mL×3). The combined organic phase was washed with brine, dried over sodium sulfate, filtered and concentrated under vacuum. The residue was purified by silica gel column chromatography eluting with ethyl acetate in petroleum from 0% to 50% to afford benzyl N-ethyl-N-[(3S)-1-(2-methylpropanoyl)pyrrolidin-3-yl]carbamate (7.73 g, 24.29 mmol, 84.37% yield) as a yellow oil. LCMS ESI (+) m/z 319.1 (M+H).

Step I: Preparation of 1-[(3S)-3-(ethylamino)pyrrolidin-1-yl]-2-methyl-propan-1-one: To a solution of benzyl N-ethyl-N-[(3S)-1-(2-methylpropanoyl)pyrrolidin-3-yl]carbamate (1.00 eq, 7.73 g, 24.29 mmol) in THF (100 mL) was added Pd/C (2.40 g) and Pd(OH)₂ (2.4 g). The resulting mixture was recharged with nitrogen gas three times and then recharged with hydrogen gas three times. The resulting mixture was stirred at room temperature for 16 h under hydrogen atmosphere. The reaction was monitored by LCMS. The resulting mixture was filtered and the filtrate was concentrated under vacuum to afford 1-[(3S)-3-(ethylamino)pyrrolidin-1-yl]-2-methyl-propan-1-one (4.19 g, 22.75 mmol, 93.66% yield) as a yellow oil, which was used in next step directly without further purification. LCMS ESI (+) m/z 185.1 (M+H).

Step J: Preparation of tert-butyl N-[3-cyano-4-[4-[ethyl-[(3S)-1-(2-methylpropanoyl)pyrrolidin-3-yl]amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophen-2-yl]carbamate: To a solution of tert-butyl (4-((S)-4-chloro-8-fluoro-2-(((2R,7aS)-2-fluorotetrahydro-1H-pyrrolizin-7a(5H)-yl)methoxy)-6-(trifluoromethyl)quinazolin-7-yl)-3-cyano-7-fluorobenzo[b]thiophen-2-yl)carbamate (1.00 eq, 7.93 g, 11.38 mmol) in dioxane (80 mL) was added DIPEA (3.00 eq. 4.4 g, 34.13 mmol) and 1-[(3S)-3-(ethylamino)pyrrolidin-1-yl]-2-methyl-propan-1-one (2.00 eq, 4.19 g, 22.75 mmol) at 25° C. under nitrogen atmosphere. The resulting mixture was stirred at 25° C. for 16 h under nitrogen atmosphere. The reaction was monitored by LCMS. The resulting mixture was diluted with water and extracted with ethyl acetate (100 mL×3). The combined organic phase was washed with brine, dried over sodium sulfate, filtered and concentrated under vacuum. The residue was purified by silica gel column chromatography eluting with MeOH in DCM from 0.5% to 4% to afford tert-butyl N-[3-cyano-4-[[4-[ethyl-[(3S)-1-(2-methylpropanoyl)pyrrolidin-3-yl]amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophen-2-yl]carbamate (7.53 g, 8.91 mmol, 78.30% yield) as a yellow solid. LCMS ESI (+) m/z 846.1 (M+H).

Step K: Preparation of 2-amino-4-{4-[ethyl-[(3S)-1-(2-methylpropanoyl)pyrrolidin-3-yl]amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile: To a solution of tert-butyl N-{3-cyano-4-[4-[ethyl-[(3S)-1-(2-methylpropanoyl)pyrrolidin-3-yl]amino]-8-fluoro-2-{[(2R,8S)-2-fluoro-1,2,3,5,6,7-hexahydropyrrolizin-8-yl]methoxy]-6-(trifluoromethyl)quinazolin-7-yl]-7-fluoro-benzothiophen-2-yl]carbamate (1.00 eq, 7.53 g, 8.91 mmol) in DCM (60 mL) was added TFA (35.4 eq, 20 mL, 260 mmol) at 25° C. under nitrogen atmosphere. The resulting mixture was stirred at 25° C. for 2 h under nitrogen atmosphere. The reaction was monitored by LCMS. Tc resulting mixture was concentrated under vacuum. The residue was adjusted to pH=8 with saturated sodium bicarbonate aqueous solution and extracted with ethyl acetate (100 mL×3). The combined organic phase was washed with water and saturated brine solution, dried sodium sulfate, filtered and concentrated under vacuum. The residue was purified by silica gel column chromatography eluting with MeOH in DCM from 0.5% to 6% to afford 2-amino-4-{4-{ethyl-{(3S)-1-(2-methylpropanoyl)pyrrolidin-3-yl]amino]-8-fluoro-2-{[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (4.20 g, 5.64 mmol, 63.30% yield). LCMS ESI (+) m/z 746.1 (M+H). ¹H NMR (400 MHz, CD₃OD) δ 8.24 (d, 1H), 7.26-7.17 (m, 1H), 7.01 (t, 1H), 5.37 (s, 0.5H), 5.24 (s, 0.5H), 5.07-5.03 (m, 1H), 4.39-4.21 (m, 2H), 4.06-3.36 (m, 6H), 3.23 (s, 3H), 3.03 (s, 1H), 2.91-2.74 (m, 1H), 2.57-2.10 (m, 5H), 2.02-1.98 (m, 2H), 1.96-1.76 (m, 1H), 1.42 (q, 3H), 1.14 (d, 6H).

### Synthetic Example 23: 2-amino-4-((S)-4-(ethyl((6S,7aS)-3-oxotetrahydro-1H,3H-pyrrolo[1,2-c]oxazol-6-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile (Compound 518)

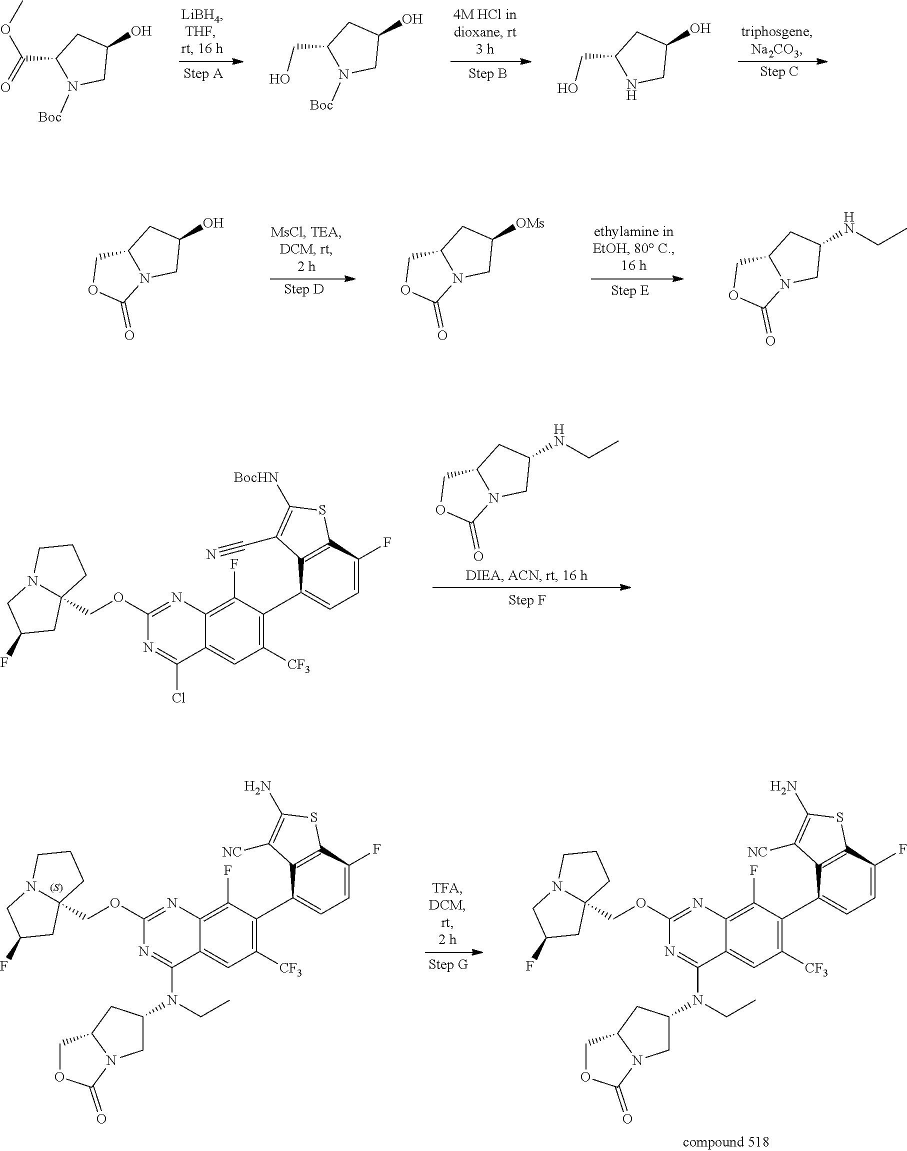

Step A: Preparation of tert-butyl (2S,4R)-4-hydroxy-2-(hydroxymethyl)pyrrolidine-1-carboxylate: To a solution of 1-(tert-butyl) 2-methyl (2S,4R)-4-hydroxypyrrolidine-1,2-dicarboxylate (1.00 eq, 5.0 g, 20.4 mmol) in ThF (50 mL) was added LiBH₄ (5.00 eq, 2.24 g, 62.0 mmol) in portions at 0° C. under nitrogen atmospheric. The resulting mixture was stirred at 25° C. for 16 h under nitrogen atmosphere. The reaction was monitored by LCMS. The resulting mixture was diluted with water (100 mL) and extracted with ethyl acetate (100 mL×3). The combined organic phase was washed with brine (200 mL), dried over anhydrous sodium sulfate and concentrated under vacuum. The residue was purified by silica gel column chromatography eluting with ethyl acetate in petroleum from 0% to 50% to afford tert-butyl (2S,4R)-4-hydroxy-2-(hydroxymethyl)pyrrolidine-1-carboxylate (3.30 g, 15.2 mmol, 74.51% yield) as a yellow solid. LCMS ESI (+) m/z 218.2 (M+H).

Step B: Preparation of (3R,5S)-5-(hydroxymethyl)pyrrolidin-3-ol: A solution of tert-butyl (2S,4R)-4-hydroxy-2-(hydroxymethyl)pyrrolidine-1-carboxylate (1.00 eq, 3.0 g, 13.8 mmol) in 4M hydrochloric acid of dioxane (30 mL) was stirred at 25° C. for 3 h under nitrogen atmosphere. The reaction was monitored by LCMS. The resulting mixture was concentrated under vacuum to afford crude (3R,5S)-5-(hydroxymethyl)pyrrolidin-3-ol (3 g) as a yellow solid, which was used in next step directly without further purification. LCMS ESI (+) m/z 118.2 (M+H).

Step C: Preparation of (6R,7aS)-6-hydroxytetrahydro-1H,3H-pyrrolo[1,2-c]oxazol-3-one: To a solution of (3R,5S)-5-(hydroxymethyl)pyrrolidin-3-ol (1.00 eq, 2.0 g, 17.1 mmol) in 1M Na₂CO₃ aqueous solution (30 mL) was added dropwise a solution of triphosgene (1.60 eq, 8.1 g, 27.4 mmol) in toluene (100 mL) at 0° C. under nitrogen atmosphere. The resulting mixture was stirred at 25° C. for 16 h under nitrogen atmosphere. The reaction was monitored by LCMS. The resulting mixture was diluted with water and extracted with DCM (20 mL×4). The combined organic phase was washed with brine, dried over sodium sulfate, filtered and concentrated under vacuum. The residue was purified by silica gel column chromatography eluting with MeOH in DCM from 1% to 5% to afford (6R,7aS)-6-hydroxytetrahydro-1H,3H-pyrrolo[1,2-c]oxazol-3-one (670 mg, 4.68 mmol, 27.36% yield) as a yellow solid. LCMS ESI (+) m/z 144.1 (M+H).

Step D: Preparation of (6R,7aS)-3-oxotetrahydro-1H,3H-pyrrolo[1,2-c]oxazol-6-yl methanesulfonate: To a solution of (6R,7aS)-6-hydroxytetrahydro-1H,3H-pyrrolo[1,2-c]oxazol-3-one (1.00 eq. 800 mg, 5.6 mmol) in DCM (10 mL) was added TEA (3.00 eq, 1.7 g, 16.8 mmol). Then MsCl (1.20 eq, 766.1 mg, 6.7 mmol) was added to the above mixture with dropwise at 0° C. under nitrogen atmosphere. The resulting mixture was stirred at 25° C. for 2 h nitrogen atmosphere. The reaction was monitored by LCMS. The resulting mixture was diluted with water and extracted with DCM (20 mL×3). The combined organic phase was washed with brine, dried over sodium sulfate, filtered and concentrated under vacuum to afford crude (6R,7aS)-3-oxotetrahydro-1H,3H-pyrrolo[1,2-c]oxazol-6-yl methanesulfonate (800 mg) as a yellow oil, which was used in next step directly without further purification. LCMS ESI (+) m/z 222.3 (M−H).

Step E: Preparation of (6S,7aS)-6-(ethylamino)tetrahydro-1H,3H-pyrrolo[1,2-c]oxazol-3-one: A solution of (6R,7aS)-3-oxotetrahydro-1H,3H-pyrrolo[1,2-c]oxazol-6-yl methanesulfonate (1.00 eq, 800 mg, 3.6 mmol) in 10% ethylamine of EtOH solution (10 mL) was stirred at 80° C. for 16 h. The reaction was monitored by LCMS. The resulting mixture was concentrated under vacuum. The residue was purified by silica gel column chromatography eluting with MeOH in DCM from 2% to 10% to afford (6S,7aS)-6-(ethylamino)tetrahydro-1H,3H-pyrrolo[1,2-c]oxazol-3-one (400 mg, 2.3 mmol, 65.36% yield) as a yellow solid. LCMS ESI (+) m/z 171.2 (M+H).

Step F: Preparation of tert-butyl (3-cyano-4-((S)-4-(ethyl((6S,7aS)-3-oxotetrahydro-1H,3H-pyrrolo[1,2-c]oxazol-6-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophen-2-yl)carbamate: To a solution of tert-butyl (4-((S)-4-chloro-8-fluoro-2-(((2R,7aS)-2-fluorotetrahydro-1H-pyrrolizin-7a(5H)-yl)methoxy)-6-(trifluoromethyl)quinazolin-7-yl)-3-cyano-7-fluorobenzo[b]thiophen-2-yl)carbamate (1.00 eq, 50 mg, 0.071 mmol) in acetonitrile (3 mL) was added (6S,7aS)-6-(ethylamino)tetrahydro-1H,3H-pyrrolo[1,2-c]oxazol-3-one (2.00 eq, 24.1 mg, 0.142 mmol) and DIEA (5.00 eq, 45.8 mg, 0.355 mmol) at 25° C. under nitrogen atmosphere. The resulting mixture was stirred at 25° C. for 16 h under nitrogen atmosphere. The reaction was monitored by LCMS. The resulting mixture was diluted with water and extracted with ethyl acetate (5 mL×3). The combined organic phase was washed with brine, dried over sodium sulfate, filtered and concentrated under vacuum. The residue was purified by prep-TLC (DCM:MeOH=15:1) to afford tert-butyl (3-cyano-4-((S)-4-(ethyl((6S,7aS)-3-oxotetrahydro-1H,3H-pyrrolo[1,2-c]oxazol-6-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophen-2-yl)carbamate (30 mg, 0.036 mmol, 50.84% yield) as a yellow solid. LCMS ESI (+) m/z 832.3 (M+H).

Step G: Preparation of 2-amino-4-((S)-4-(ethyl((6S,7aS)-3-oxotetrahydro-1H,3H-pyrrolo[1,2-c]oxazol-6-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile: To a solution of tert-butyl (3-cyano-4-((S)-4-(ethyl((6S,7aS)-3-oxotetrahydro-1H,3H-pyrrolo[1,2-c]oxazol-6-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophen-2-yl)carbamate (1.00 eq, 30 mg, 0.036 mmol) in DCM (3 mL) was added TFA (1.0 mL) at 0° C. under nitrogen atmosphere. The resulting mixture was stirred at 25° C. for 2 h under nitrogen atmosphere. The reaction was monitored by LCMS. The reaction was quenched with saturated NaHCO₃ aqueous solution and extracted with DCM (5 mL×3). The combined organic phase was washed with brine, dried over sodium sulfate, filtered and concentrated under vacuum. The residue was purified by prep-TLC (DCM:MeOH=10:1) to afford 2-amino-4-((S)-4-(ethyl((6S,7aS)-3-oxotetrahydro-1H,3H-pyrrolo[1,2-c]oxazol-6-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile (11.8 mg, 0.016 mmol, 44.84% yield) as a white solid. LCMS ESI (+) m/z 732.2 (M+H). ¹H NMR (400 MHz, CD₃OD) δ 8.18 (s, 1H), 7.22-7.19 (m, 1H), 7.03-6.98 (m, 1H), 5.39 (s, 0.5H), 5.26 (s, 0.5H), 5.01-4.97 (m, 1H), 4.63-4.59 (m, 1H), 4.40-4.37 (m, 1H), 4.32-4.21 (m, 3H), 3.86-3.61 (m, 4H), 3.45 (s, 1H), 3.25-3.23 (m, 2H), 3.06 (s, 1H), 2.45-2.18 (m, 5H), 2.02-1.91 (m, 3H), 1.50-1.46 (m, 3H).

### Synthetic Example 24: 2-amino-4-(4-(ethyl((S)-1-ethyl-5-oxopyrrolidin-3-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile: (Compound 529)

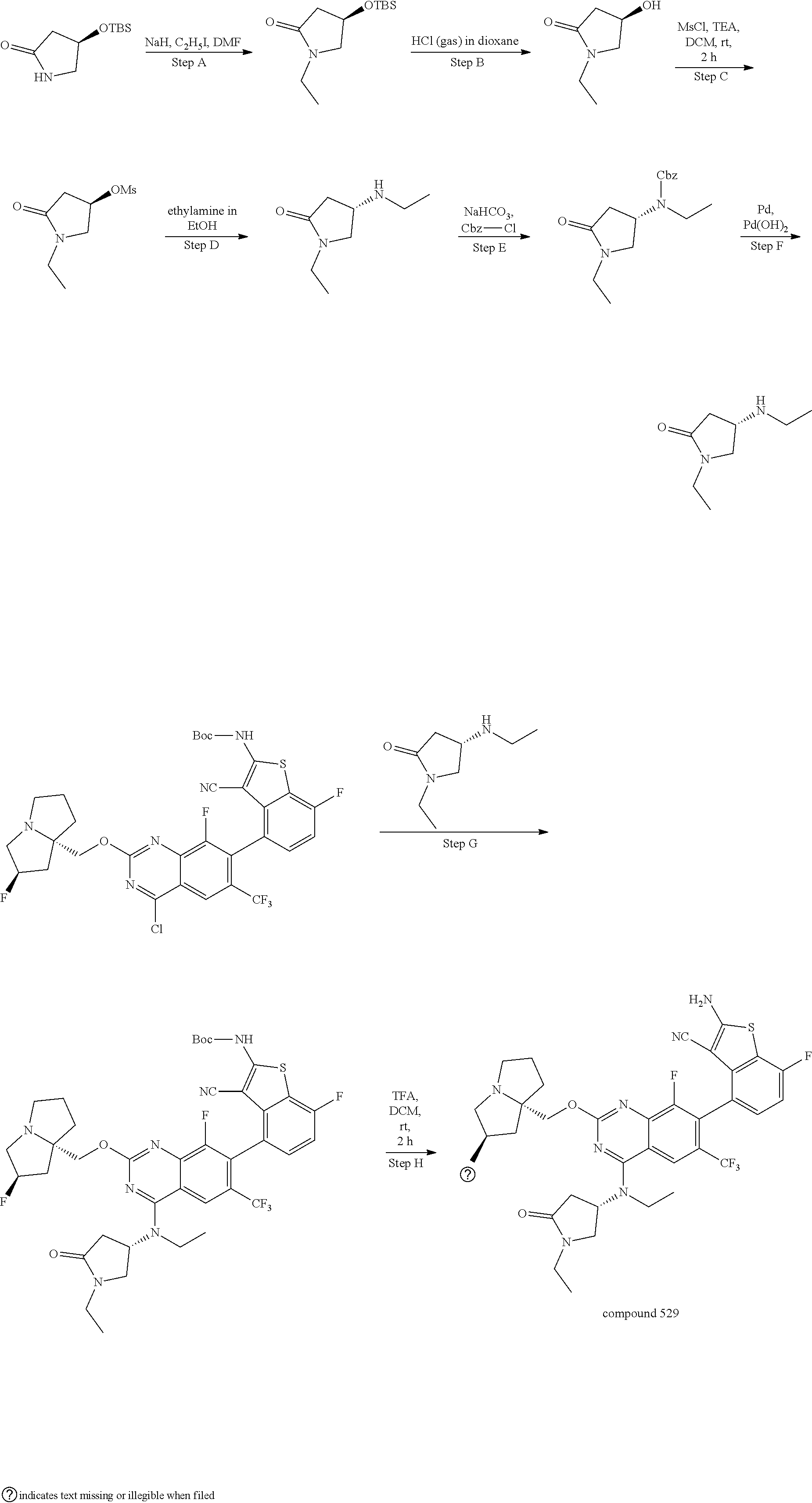

Step A: Preparation of(R)-4-((tert-butyldimethylsilyl)oxy)-1-ethylpyrrolidin-2-one: To DMF (10 mL) was added NaH (1.50 eq (0.33 g, 13.93 mmol) at 0° C. under nitrogen atmosphere. Then a solution of (R)-4-((tert-butyldimethylsilyl)oxy)pyrrolidin-2-one (1.00 eq, 2.00 g, 9.28 mmol) in DMF (10 mL) was added to above mixture dropwise at 0° C. under nitrogen atmosphere. The resulting mixture was stirred at 25° C. for 1 h under nitrogen atmosphere. Then C₂H₅I (2.00 eq, 2.97 g, 19.0 mmol) was added dropwise at 0° C. under nitrogen atmosphere. The resulting mixture was stirred at 25° C. for 16 h under nitrogen atmosphere. The reaction was monitored by LCMS. The reaction was quenched with ice-water and extracted with ethyl acetate (20 mL×4). The combined organic phase was washed with brine, dried over sodium sulfate, filtered and concentrated under vacuum. The residue was purified by silica gel column chromatography eluting with EA in petroleum from 0% to 20% to afford (R)-4-((tert-butyldimethylsilyl)oxy)-1-ethylpyrrolidin-2-one (863 mg, 3.55 mmol, 38.19% yield) as yellow oil. LCMS ESI (+) m/z 244.4 (M+H).

Step B: Preparation of (R)-1-ethyl-4-hydroxypyrrolidin-2-one: A solution of (R)-4-((tert-butyldimethylsilyl)oxy)yl)-1-ethylpyrrolidin-2-one (1.00 eq. 863 mg, 3.55 mmol) in HC (gas) in dioxane (6 mL, 4N) was stirred at 25° C. for 1 h under nitrogen atmosphere. The reaction was monitored by LCMS. The solution was concentrated under vacuum to afford crude (R)-1-ethyl-4-hydroxypyrrolidin-2-one (614 mg, 4.75 mmol, 134% yield) as a yellow oil, which was used in next step directly without further purification. LCMS ESI (+) m/z 130.1 (M+H).

Step C: Preparation of (R)-1-ethyl-5-oxopyrrolidin-3-yl methanesulfonate: To a solution of (R)-1-ethyl-4-hydroxypyrrolidin-2-one (1.00 eq, 614 mg, 4.75 mmol) in DCM (6 mL) was added TEA (3.01 eq, 2.00 mL, 14.3 mmol). The resulting mixture was cooled to 0° C. and then methanesulfonyl chloride (2.00 eq, 1.1 g, 9.50 mmol) was added dropwise at 0° C. under nitrogen atmosphere. The resulting mixture was stirred at room temperature for 2 h under nitrogen atmosphere. The reaction was monitored by LCMS. The reaction was quenched with water, extracted with DCM (10 mL×3). The combined organic phase was washed with brine (10 mL), dried over anhydrous sodium sulfate and concentrated under vacuum to afford (R)-1-ethyl-5-oxopyrrolidin-3-yl methanesulfonate (850 mg, 4.10 mmol, 86.27% yield) as a yellow oil, which was used in next step directly without further purification. LCMS ESI (+) m/z 208.0 (M+H).

Step D: Preparation of (S)-1-ethyl-4-(ethylamino)pyrrolidin-2-one: To a solution of (R)-1-ethyl-5-oxopyrrolidin-3-yl methanesulfonate (1.00 eq, 850 mg, 4.10 mmol) in EtOH was added ethylamine in THF (9.0 mL, 4N) at 25° C. The resulting mixture was stirred at 140° C. for 16 h. The reaction was monitored by LCMS. The resulting mixture was concentrated under vacuum. The residue was purified by silica gel column chromatography eluting with MeOH in DCM from 0% to 5% to afford purified chromatography to give (S)-1-ethyl-4-(ethylamino)pyrrolidin-2-one (535 mg, 3.43 mmol, 83.59% yield) as a yellow solid. LCMS ESI (+) m/z 157.1 (M+H).

Step E: Preparation of benzyl (S)-ethyl(1-ethyl-5-oxopyrrolidin-3-yl) carbamate: To a solution of (S)-1-ethyl-4-(ethylamino) pyrrolidin-2-one (1.00 eq, 420 mg, 4.10 mmol) in THF (4 mL) and H₂O (4.0 mL) was added NaHCO₃ (4.00 eq, 903.4 mg, 10.8 mmol). Then Cbz-Cl (2.00 eq, 917 mg, 5.38 mmol) was added dropwise at 0° C. under nitrogen atmosphere. The reaction mixture was stirred at 25° C. for 16 h under nitrogen atmosphere. The reaction was monitored by LCMS. The reaction was quenched with water and extracted with ethyl acetate (10 mL×4). The combined organic phase was washed with brine, dried over sodium sulfate and concentrated under vacuum. The residue was purified by silica gel column chromatography eluting with EA in petroleum from 0% to 50% to afford benzyl N-ethyl-N-[(3S)-1-ethyl-5-oxo-pyrrolidin-3-yl]carbamate (178 mg, 0.472 mmol, 17.56% yield) as a red oil. LCMS ESI (+) m/z 291.1 (M+H).

Step F: Preparation of (S)-1-ethyl-4-(ethylamino)pyrrolidin-2-one: To a solution of benzyl (S)-ethyl(1-ethyl-5-oxopyrrolidin-3-yl) carbamate (1.00 eq, 178 mg, 0.472 mmol) in THF (2 mL) was added Pd/C (18 mg) and Pd(OH)₂ (18 mg). The reaction mixture was stirred at 25° C. for 16 h under hydrogen atmosphere. The reaction was monitored by LCMS. The resulting mixture was filtered and the filtrate was concentrated under reduced pressure to afford (S)-1-ethyl-4-(ethylamino)pyrrolidin-2-one (70 mg, 22.75 mmol, 73.09% yield) as a yellow oil, which was used in next step directly without further purification. LCMS ESI (+) m/z 157.1 (M+H).

Step G: Preparation of tert-butyl (3-cyano-4-(4-(ethyl((S)-1-ethyl-5-oxopyrrolidin-3-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophen-2-yl)carbamate: To a solution of tert-butyl (4-(4-chloro-8-fluoro-2-(((2R,7aS)-2-fluorotetrahydro-1H-pyrrolizin-7a(5H)-yl)methoxy)-6-(trifluoromethyl)quinazolin-7-yl)-3-cyano-7-fluorobenzo[b]thiophen-2-yl)carbamate (1.00 eq, 73 mg, 0.1045 mmol) in acetonitrile (2 mL) was added DIPEA (5.00 eq, 67.6 mg, 0.523 mmol) and (S)-1-ethyl-4-(ethylamino)pyrrolidin-2-one (3.00 eq, 49.01 mg, 0.314 mmol) at 30° C. under nitrogen atmosphere. The resulting mixture was stirred at 30° C. for 16 h under nitrogen atmosphere. The reaction was monitored by LCMS. The reaction mixture was diluted with water and extracted with ethyl acetate (10 mL×3). The combined organic phase was washed with brine, dried over sodium sulfate, filtered and concentrated under vacuum. The residue was purified by Prep-TLC (DCM:MeOH=20:1) to afford tert-butyl (3-cyano-4-(4-(ethyl((S)-1-ethyl-5-oxopyrrolidin-3-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophen-2-yl)carbamate (40 mg, 0.0435 mmol, 50.64% yield) as a yellow solid. LCMS ESI (+) m/z 818.2 (M+H).

Step H: Preparation of 2-amino-4-(4-(ethyl((S)-1-ethyl-5-oxopyrrolidin-3-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile: To a solution of tert-butyl (3-cyano-4-(4-(ethyl((S)-1-ethyl-5-oxopyrrolidin-3-yl)amino)-1-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophen-2-yl)carbamate (1.00 eq, 40 mg, 0.0435 mmol) in DCM (2 mL) was added TFA (0.5 mL) at 25° C. The resulting mixture was stirred at 25° C. for 2 h under nitrogen atmosphere. The reaction was monitored by LCMS. The mixture was concentrated under vacuum and the residue was adjusted to pH=8 with saturated sodium bicarbonate aqueous solution, extracted with ethyl acetate (10 mL×3), washed with water and brine, dried over sodium sulfate, filtered and concentrated under vacuum. The residue was purified by Prep-HPLC to afford 2-amino-4-(4-(ethyl((S)-1-ethyl-5-oxopyrrolidin-3-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile (8.3 mg, 0.0115 mmol, 43.30% yield) as an off-white solid. LCMS ESI (+) m/z 718.1 (M+H). ¹H NMR (400 MHz, CD₃OD) δ 8.18 (s, 11H), 7.23-7.20 (s, 1H), 7.01 (t, 1H), 5.48 (s, 0.5H), 5.37 (s, 0.5H), 4.75-4.69 (m, 1H), 4.39-4.29 (m, 2H), 3.96-3.83 (m, 3H), 3.77-3.71 (m, 1H), 3.63-3.47 (m, 3H), 3.25-3.19 (m, 1H), 3.01-2.95 (m, 1H), 2.88-2.81 (m, 1H), 2.50-2.25 (m, 4H), 3.14-1.93 (m, 4H), 1.54 (t, 3H), 1.20 (t, 3H).

### Synthetic Example 25: (3S)-3-[[7-(2-amino-3-cyano-7-fluoro-benzothiophen-4-yl)-8-fluoro-2-[[(2R,8S)-2-fluoro-1,2,3,5,6,7-hexahydropyrrolizin-8-yl]methoxy]-6-(trifluoromethyl)quinazolin-4-yl]-ethyl-amino]-N,N-dimethyl-pyrrolidine-1-carboxamide (Compound 540)

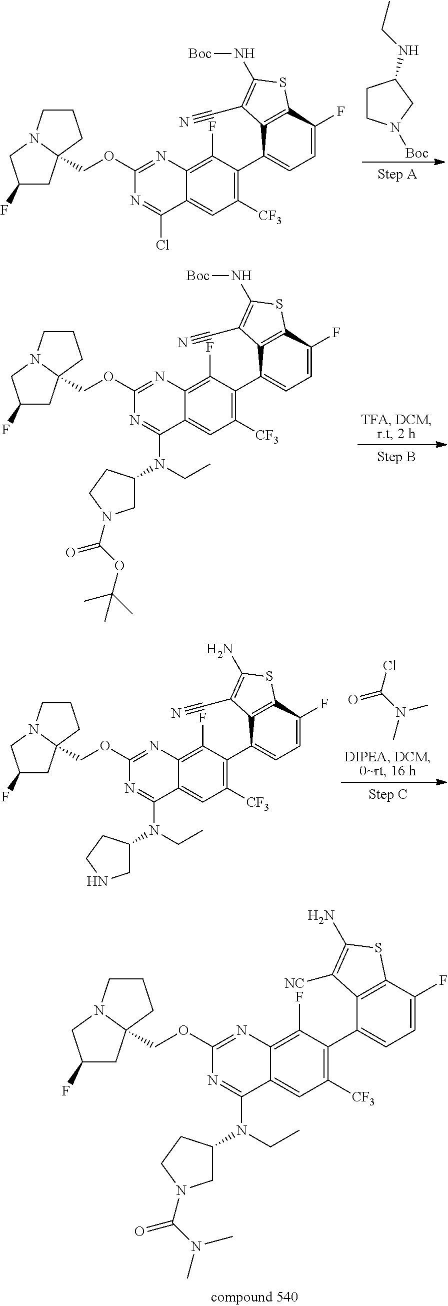

Step A: Preparation of tert-butyl (3S)-3-[[7-[2-(tert-butoxycarbonylamino)-3-cyano-7-fluoro-benzothiophen-4-yl]-8-fluoro-2-[[(2R,8S)-2-fluoro-1,2,3,5,6,7-hexahydropyrrolizin-8-yl]methoxy]-6-(trifluoromethyl)quinazolin-4-yl]-ethyl-amino]pyrrolidine-1-carboxylat: To a solution of tert-butyl N-[4-[4-chloro-8-fluoro-2-[[(2R,8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate (1.00 eq, 2.00 g, 2.87 mmol) in 1,4-dioxane (30 mL) was added DIPEA (3.00 eq, 870 mg, 8.60 mmol) and tert-butyl (3S)-3-(ethylamino)pyrrolidine-1-carboxylate (1.50 eq, 921 mg, 4.30 mmol) at 25° C. under nitrogen atmosphere. The resulting mixture was stirred at 45° C. for 16 h under nitrogen atmosphere. The reaction was monitored by LCMS. The resulting mixture was diluted with water and extracted with ethyl acetate (50 mL×5). The combined organic phase was washed with brine, dried over sodium sulfate, filtered and concentrated under vacuum. The residue was purified by silica gel column chromatography eluting with MeOH in DCM from 0.5% to 5% to afford tert-butyl (3S)-3-[[7-[2-(tert-butoxycarbonylamino)-3-cyano-7-fluoro-benzothiophen-4-yl]-8-fluoro-2-[[(2R,8S)-2-fluoro-1,2,3,5,6,7-hexahydropyrrolizin-8-yl]methoxy]-6-(trifluoromethyl)quinazolin-4-yl]-ethyl-amino]pyrrolidine-1-carboxylate (2.00 g, 1.96 mmol, 68.54% yield) as a yellow solid. LCMS ESI (+) m/z 876.3 (M+H).

Step B: Preparation of 2-amino-4-[4-[ethyl-[(3S)-pyrrolidin-3-yl aminol-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile: To a solution of tert-butyl (3S)-3-[[7-[2-(tert-butoxycarbonylamino)-3-cyano-7-fluoro-benzothiophen-4-yl]-8-fluoro-2-{[(2R,8S)-2-fluoro-1,2,3,5,6,7-hexahydropyrrolizin-8-yl]methoxy]-6-(trifluoromethyl)quinazolin-4-yl]-ethyl-amino]pyrrolidine-1-carboxylate (1.00 eq, 110 mg, 0.1256 mmol) in DCM (4 mL) was added TFA (235 eq, 1.0 mL, 13.4 mmol) at 25° C. under nitrogen atmosphere. The resulting mixture was stirred at 25° C. for 2 h under nitrogen atmosphere. The reaction was monitored by LCMS. The resulting mixture was concentrated to afford crude 2-amino-4-[4-[ethyl-[(3S)-pyrrolidin-3-yl]amino]-8-fluoro-2-{[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (110 mg, 0.130 mmol, 103.71% yield) as a yellow solid, which was used in the next step directly without further purification. LCMS ESI (+) m/z 676.1 (M+H).

Step C: Preparation of (3S)-3-[[7-(2-amino-3-cyano-7-fluoro-benzothiophen-4-yl)-8-fluoro-2-[[(2R,8S)-2-fluoro-1,2,3,5,6,7-hexahydropyrrolizin-8-yl]methoxy]-6-(trifluoromethyl)quinazolin-4-yl]-ethyl-amino]-N,N-dimethyl-pyrrolidine-1-carboxamide: To a solution of 2-amino-4-[4-[ethyl-[(3S)-pyrrolidin-3-yl]amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (1.00 eq, 110 mg, 0.163 mmol) in DCM (4 mL) was added DIPEA (5.00 eq, 82 mg, 0.814 mmol). Then a solution of dimethylcarbamic chloride (1.00 eq, 17.5 mg, 0.163 mmol) in DCM (1 mL) was added to above mixture dropwise at 0° C. under nitrogen atmosphere. The resulting mixture was stirred at 25° C. for 16 h under nitrogen atmosphere. The reaction was monitored by LCMS. The reaction was quenched with saturated sodium bicarbonate aqueous solution and extracted with ethyl acetate (10 mL×4). The combined organic phase was washed with brine, dried over sodium sulfate, filtered and concentrated under vacuum. The residue was purified by prep-TLC (DCM:MeOH=10:1) to give (3S)-3-{[7-(2-amino-3-cyano-7-fluoro-benzothiophen-4-yl)-8-fluoro-2-{[(2R,8S)-2-fluoro-1,2,3,5,6,7-hexahydropyrrolizin-8-yl]methoxy]-6-(trifluoromethyl)quinazolin-4-yl]-ethyl-amino]-N,N-dimethyl-pyrrolidine-1-carboxamide (48 mg, 0.0594 mmol, 36.48% yield) as an off-white solid. LCMS ESI (+) m/z 747.2 (M+H). ¹H NMR (400 MHz, CD₃OD) δ 8.28 (s, 1H), 7.21-7.25 (m, 1H), 7.02 (t, 1H), 6.64 (s, 2H), 5.58 (s, 0.5H), 5.45 (s, 0.5H), 5.01-5.05 (m, 1H), 4.55-4.67 (m, 2H), 3.71-3.96 (m, 6H), 3.53-3.65 (m, 3H), 3.34-3.41 (m, 1H), 2.89 (s, 6H), 2.27-2.67 (m, 7H), 2.09-2.15 (m, 1H), 1.47 (t, 3H).

### Synthetic Example 26: methyl (3S)-3-[[7-(2-amino-3-cyano-7-fluoro-benzothiophen-4-yl)-8-fluoro-2-[[(2R,8S)-2-fluoro-1,2,3,5,6,7-hexahydropyrrolizin-8-yl]methoxy]-6-(trifluoromethyl) quinazolin-4-yl]-ethyl-amino]pyrrolidine-1-carboxylate (Compound 541)

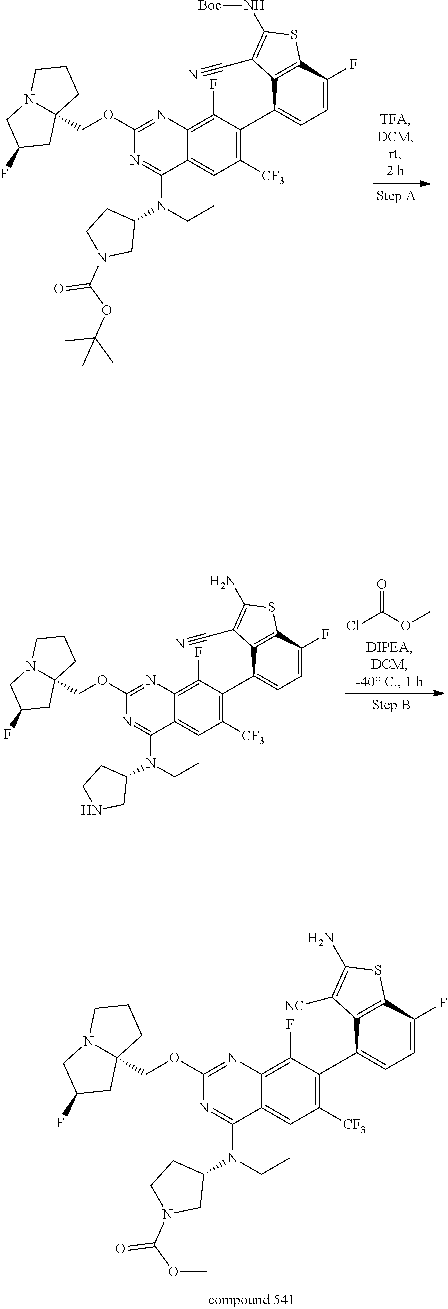

Step A: Preparation of 2-amino-4-[4-[ethyl-[(3S)-pyrrolidin-3-yl]amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile: To a solution of tert-butyl (3S)-3-[[7-[2-(tert-butoxycarbonylamino)-3-cyano-7-fluoro-benzothiophen-4-yl]-8-fluoro-2-[[(2R,8S)-2-fluoro-1,2,3,5,6,7-hexahydropyrrolizin-8-yl]methoxy]-6-(trifluoromethyl)quinazolin-4-yl]-ethyl-amino]pyrrolidine-1-carboxylate (1.00 eq, 130 mg, 0.1484 mmol) in DCM (4 mL) was added TFA (235 eq, 1.0 mL, 13.4 mmol) at 25° C. under nitrogen atmosphere. The resulting mixture was stirred at 25° C. for 2 h under nitrogen atmosphere. The reaction was monitored by LCMS. The resulting mixture was concentrated under vacuum to afford crude 2-amino-4-[4-[ethyl-[(3S)-pyrrolidin-3-yl]amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (130 mg, 0.154 mmol) as a yellow solid, which was used in the next step directly without further purification. LCMS ESI (+) m/z 676.1 (M+H).

Step B: Preparation of methyl (3S)-3-[[7-(2-amino-3-cyano-7-fluoro-benzothiophen-4-yl)-8-fluoro-2-[[(2R,8S)-2-fluoro-1,2,3,5,6,7-hexahydropyrrolizin-8-yl]methoxy-6-(trifluoromethyl)quinazolin-4-yl]-ethyl-amino]pyrrolidine-1-carboxylate: To a solution of 2-amino-4-[4-[ethyl-[(3S)-pyrrolidin-3-yl]amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (1.00 eq, 130 mg, 0.192 mmol) in DCM (4 mL) was added DIEA (5.00 eq, 82 mg, 0.814 mmol). Then a solution of methyl carbonochloridate (1.00 eq, 18.182 mg, 0.192 mmol) in DCM (1 mL) was added to above mixture dropwise at −40° C. under nitrogen atmosphere. The resulting mixture was stirred at −40° C. for 1 h under nitrogen atmosphere. The reaction was monitored by LCMS. The reaction was quenched with saturated sodium bicarbonate aqueous solution and extracted with ethyl acetate (10 mL×4). The combined organic phase was washed with brine, dried over sodium sulfate, filtered and concentrated under vacuum. The residue was purified by prep-TLC (DCM:MeOH=10:1) to afford methyl (3S)-3-[[7-(2-amino-3-cyano-7-fluoro-benzothiophen-4-yl)-8-fluoro-2-[[(2R,8S)-2-fluoro-1,2,3,5,6,7-hexahydropyrrolizin-8-yl]methoxy]-6-(trifluoromethyl)quinazolin-4-yl]-ethyl-amino]pyrrolidine-1-carboxylate (57 mg, 0.0714 mmol, 46.49% yield) as an off-white solid. LCMS ESI (+) m/z 734.1 (M+H). ¹H NMR (400 MHz, CD₃OD) δ 8.26 (s, 1H), 7.23-7.20 (m, 1H), 7.02 (t, 1H), 6.58-6.64 (m, 2H), 5.58 (s, 0.5H), 5.45 (s, 0.5H), 5.02-5.04 (m, 1H), 4.54-4.61 (m, 2H), 3.72-3.90 (m, 9H), 3.37-3.63 (m, 3H), 3.21-3.25 (m, 1H), 2.27-2.67 (m, 7H), 2.09 (s, 1H), 1.46-1.47 (m, 3H).

### Synthetic Example 27: 2-amino-4-((S)-4-(ethyl((6S,7aS)-2-methyl-3-oxohexahydro-1H-pyrrolo[1,2-c]imidazol-6-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile (Compound 543)

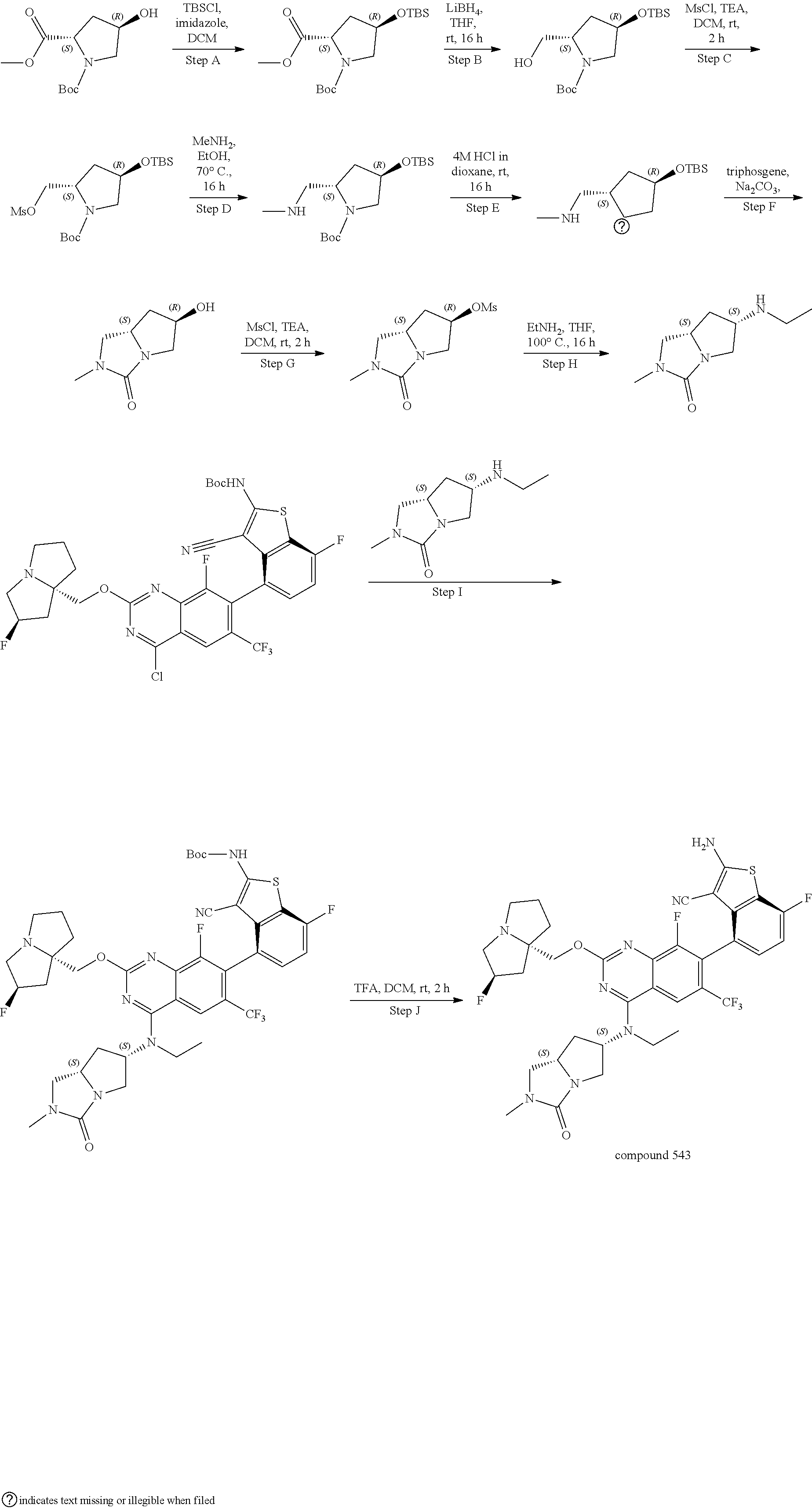

Step A: Preparation of 1-(tert-butyl) 2-methyl (2S,4R)-4-((tert-butyldimethylsilyl)oxy)pyrrolidine-1,2-dicarboxylate: To a solution of 1-(tert-butyl) 2-methyl (2S,4R)-4-hydroxypyrrolidine-1,2-dicarboxylate (1.00 eq, 25.00 g, 102 mmol) in DCM (250 mL) was added imidazole (2.00 eq, 13.88 g, 204 mmol) at 25° C. under nitrogen atmosphere. Then TBSCl (1.20 eq, 18.44 g, 122 mmol) was added to the above mixture at 0° C. under nitrogen atmosphere. The resulting mixture was stirred at 25° C. for 16 h under nitrogen atmosphere. The reaction was monitored by LCMS. The resulting mixture was diluted with water and extracted with DCM (200 mL×3). The combined organic phase was washed with brine, dried over sodium sulfate, filtered and concentrated under vacuum. The residue was purified by silica gel column chromatography eluting with MeOH in DCM from 1% to 5% to afford 1-(tert-butyl) 2-methyl (2S,4R)-4-((tert-butyldimethylsilyl)oxy)pyrrolidine-1,2-dicarboxylate (40.00 g, 90.1 mmol, 88.41% yield) as a yellow oil. LCMS ESI (+) m/z 360.2 (M+H).

Step B: Preparation of tert-butyl (2S,4R)-4-((tert-butyldimethylsilyl)oxy)-2-(hydroxymethyl)pyrrolidine-1-carboxylate: To a solution of 1-(tert-butyl) 2-methyl (2S,4R)-4-((tert-butyldimethylsilyl)oxy)pyrrolidine-1,2-dicarboxylate (1.00 eq, 23.00 g, 64.0 mmol) in THF (230 mL) was added LiBH₄ (3.00 eq, 4.18 g, 192 mmol) in portions at 0° C. under nitrogen atmosphere. The resulting mixture was stirred at 25° C. for 16 h under nitrogen atmosphere. The reaction was monitored by LCMS. The resulting mixture was diluted with water and extracted with DCM (200 mL×3). The combined organic phase was washed with brine, dried over sodium sulfate, filtered and concentrated under vacuum to afford crude tert-butyl (2S,4R)-4-((tert-butyldimethylsilyl)oxy)-2-(hydroxymethyl)pyrrolidine-1-carboxylate (19.60 g, 53.2 mmol, 83.18% yield), which was used in next step directly without further purification. LCMS ESI (+) m/z 332.3 (M+H).

Step C: Preparation of tert-butyl (2S,4R)-4-((tert-butyldimethylsilyl)oxy)-2-(((methylsulfonyl)oxy)methyl) pyrrolidine-1-carboxylate: To a solution of tert-butyl (2S,4R)-4-((tert-butyldimethylsilyl)oxy)-2-(hydroxymethyl)pyrrolidine-1-carboxylate (1.00 eq, 11.5 g, 34.7 mmol) in DCM (120 mL) was added TEA (3.00 eq, 10.5 g, 104.2 mmol) at 25° C. Then MsCl (1.20 eq, 4.7 g, 41.6 mmol) was added to the above mixture dropwise at 0° C. under nitrogen atmosphere. The resulting mixture was stirred at 25° C. for 2 h under nitrogen atmosphere. The reaction was monitored by LCMS. The resulting mixture was diluted with water and extracted with DCM (20 mL×4). The combined organic phase was washed with brine, dried over sodium sulfate, filtered and concentrated under vacuum to afford crude tert-butyl (2S,4R)-4-((tert-butyldimethylsilyl)oxy)-2-(((methylsulfonyl)oxy)methyl) pyrrolidine-1-carboxylate (13.5 g) as a yellow oil. LCMS ESI (+) m/z 410.3 (M+H).

Step D: Preparation of tert-butyl (2S,4R)-4-((tert-butyldimethylsilyl)oxy)-2-((methylamino)methyl) pyrrolidine-1-carboxylate: A solution of tert-butyl (2S,4R)-4-((tert-butyldimethylsilyl)oxy)-2-(((methylsulfonyl)oxy)methyl) pyrrolidine-1-carboxylate (1.00 eq, 14.5 g, 35.4 mmol) in 30% methylamine of ethanol (100 mL) was stirred at 70° C. for 16 h. The reaction was monitored by LCMS. The resulting mixture was concentrated under vacuum and the residue was purified by silica gel column chromatography eluting with MeOH in DCM from 1% to 5% to afford tert-butyl (2S,4R)-4-((tert-butyldimethylsilyl)oxy)-2-((methylamino)methyl) pyrrolidine-1-carboxylate (11.3 g, 32.8 mmol, 92.6% yield) as a yellow solid. LCMS ESI (+) m/z 345.2 (M+H).

Step E: Preparation of (3R,5S)-5-((methylamino)methyl)pyrrolidin-3-ol: A solution of tert-butyl (2S,4R)-4-((tert-butyldimethylsilyl)oxy)-2-((methylamino)methyl) pyrrolidine-1-carboxylate (1.00 eq, 5.0 g, 14.5 mmol) in 4M hydrochloric acid of dioxane (50 mL) was stirred at 25° C. for 16 h under nitrogen atmosphere. The reaction was monitored by LCMS. The resulting mixture was concentrated under vacuum to afford crude (3R,5S)-5-((methylamino)methyl)pyrrolidin-3-ol (1.8 g, 13.8 mmol, 95.17% yield) as a yellow solid, which was used in next step directly without further purification. LCMS ESI (+) m/z 131.2 (M+H).

Step F: Preparation of (6R,7aS)-6-hydroxy-2-methylhexahydro-3H-pyrrolo[1,2-c]imidazol-3-one: To a solution of (3R,5S)-5-((methylamino)methyl)pyrrolidin-3-ol (1.00 eq, 1.5 g, 11.5 mmol) in 1M Na₂CO₃ aqueous solution (30 mL) was added a solution of triphosgene (1.60 eq, 5.5 g, 18.4 mmol) in toluene (100 mL) with dropwise at 0° C. under nitrogen atmosphere. The resulting mixture was stirred at 25° C. for 16 h under nitrogen atmosphere. The reaction was monitored by LCMS. The resulting mixture was diluted with water and extracted with DCM (20 mL×4). The combined organic phase was washed with brine, dried over sodium sulfate, filtered and concentrated under vacuum. The residue was purified by silica gel column chromatography eluting with MeOH in DCM from 1% to 5% to afford (6R,7aS)-6-hydroxy-2-methylhexahydro-3H-pyrrolo[1,2-c]imidazol-3-one (800 mg, 5.13 mmol, 44.61% yield) as a yellow solid. LCMS ESI (+) m/z 157.2 (M+H).

Step G: Preparation of (6R,7aS)-2-methyl-3-oxohexahydro-1H-pyrrolo[1,2-c]imidazol-6-ylmethanesulfonate: To a solution of (6R,7aS)-6-hydroxy-2-methylhexahydro-3H-pyrrolo[1,2-c]imidazol-3-one (1.00 eq, 600 mg, 3.8 mmol) in DCM (10 mL) was added TEA (3.00 eq, 1.2 g, 11.4 mmol). Then MsCl (1.20 eq, 524.4 mg, 4.6 mmol) was added to the above mixture dropwise at 0° C. under nitrogen atmosphere. The resulting mixture was stirred at 25° C. for 2 h under nitrogen atmosphere. The reaction was monitored by LCMS. The resulting mixture was diluted with water and extracted with DCM (20 mL×4). The combined organic phase was washed with brine, dried over sodium sulfate, filtered and concentrated under vacuum to afford crude (6R,7aS)-2-methyl-3-oxohexahydro-1H-pyrrolo[1,2-c]imidazol-6-ylmethanesulfonate (650 mg) as a yellow oil, which was used in next step directly without further purification. LCMS ESI (+) m/z 235.3 (M+H).

Step H: Preparation of (6S,7aS)-6-(ethylamino)-2-methylhexahydro-3H-pyrrolo[1,2-c]imidazol-3-one: A solution of (6R,7aS)-2-methyl-3-oxohexahydro-1H-pyrrolo[1,2-c]imidazol-6-yl methanesulfonate (1.00 eq, 650 mg, 2.8 mmol) in ethylamine solution (10 mL, 10/c ethylamine in THE) was stirred at 100° C. for 16 h. The reaction was monitored by LCMS. The resulting mixture was concentrated under vacuum. The residue was purified by silica gel column chromatography eluting with MeOH in DCM from 2% to 10% to afford (6S,7aS)-6-(ethylamino)-2-methylhexahydro-3H-pyrrolo[1,2-c]imidazol-3-one (300 mg, 1.6 mmol, 57.14% yield) as a yellow solid. LCMS ESI (+) m/z 184.2 (M+H).

Step I: Preparation of tert-butyl (3-cyano-4-((S)-4-(ethyl((6S,7aS)-2-methyl-3-oxohexahydro-1H-pyrrolo[1,2-c]imidazol-6-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophen-2-yl)carbamate: To a solution of tert-butyl (4-((S)-4-chloro-8-fluoro-2-(((2R,7aS)-2-fluorotetrahydro-1H-pyrrolizin-7a(5H)-yl)methoxy)-6-(trifluoromethyl)quinazolin-7-yl)-3-cyano-7-fluorobenzo[b]thiophen-2-yl)carbamate (1.00 eq, 100 mg, 0.143 mmol) in acetonitrile (3 mL) was added (6S,7aS)-6-(ethylamino)-2-methylhexahydro-3H-pyrrolo[1,2-c]imidazol-3-one (2.00 eq, 52.5 mg, 0.286 mmol) and DIEA (5.00 eq, 92.2 mg, 0.715 mmol) at 25° C. under nitrogen atmosphere. The resulting mixture was stirred at 25° C. for 16 h under nitrogen atmosphere. The reaction was monitored by LCMS. The resulting mixture was diluted with water and extracted with ethyl acetate (5 mL×3). The combined organic phase was washed with brine, dried over sodium sulfate, filtered and concentrated under vacuum. The residue was purified by prep-TLC (DCM:MeOH=15:1) to afford tert-butyl (3-cyano-4-((S)-4-(ethyl((6S,7aS)-2-methyl-3-oxohexahydro-1H-pyrrolo[1,2-c]imidazol-6-yl)amino)-8-fluoro-2-(((2R,7aS)-2-fluorotetrahydro-H-pyrrolizin-7a(5H)-yl)methoxy)-6-(trifluoromethyl)quinazolin-7-yl)-7-fluorobenzo[b]thiophen-2-yl)carbamate (50 mg, 0.059 mmol, 41.43% yield) as a yellow solid. LCMS ESI (+) m/z 845.3 (M+H).

Step J: Preparation of 2-anion-4-((S)-4-(ethyl((6S,7aS)-2-methyl-3-oxohexahydro-1H-pyrrolo[1,2-c]imidazol-6-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile: To a solution of tert-butyl (3-cyano-4-((S)-4-(ethyl((6S,7aS)-2-methyl-3-oxohexahydro-1H-pyrrolo[1,2-c]imidazol-6-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophen-2-yl)carbamate (1.00 eq, 40 mg, 0.047 mmol) in DCM (3 mL) was added TFA (1.0 mL) at 0° C. under nitrogen atmosphere. The resulting mixture was stirred at 25° C. for 2 h nitrogen atmosphere. The reaction was monitored by LCMS. The reaction was quenched with saturated sodium bicarbonate aqueous solution and extracted with DCM (5 mL×3). The combined organic phase was washed with brine, dried over sodium sulfate, filtered and concentrated under vacuum. The residue was purified by prep-TLC (DCM:MeOH=10:1) to afford 2-amino-4-((S)-4-(ethyl((6S,7aS)-2-methyl-3-oxohexahydro-1H-pyrrolo[1,2-c]imidazol-6-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile (24.7 mg, 0.033 mmol, 70.21% yield) as a white solid. LCMS ESI (+) m/z 745.2 (M+H). ¹H NMR (400 MHz, CD₃OD) δ 8.19 (s, 1H), 7.22-7.20 (m, 1H), 7.02-6.98 (m, 1H), 5.36 (s, 0.5H), 5.22 (s, 1.5H), 4.31-4.20 (m, 2H), 3.88 (m, 1H), 3.76-3.45 (m, 6H), 3.22-3.18 (m, 3H), 2.99 (s, 1H), 2.86 (s, 3H), 2.35-1.88 (m, 8H), 1.37 (s, 3H).

### Synthetic Example 28: 2-amino-4-((S)-4-(ethyl((2S,7aR)-5-oxohexahydro-1H-pyrrolizin-2-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile (Compound 573)

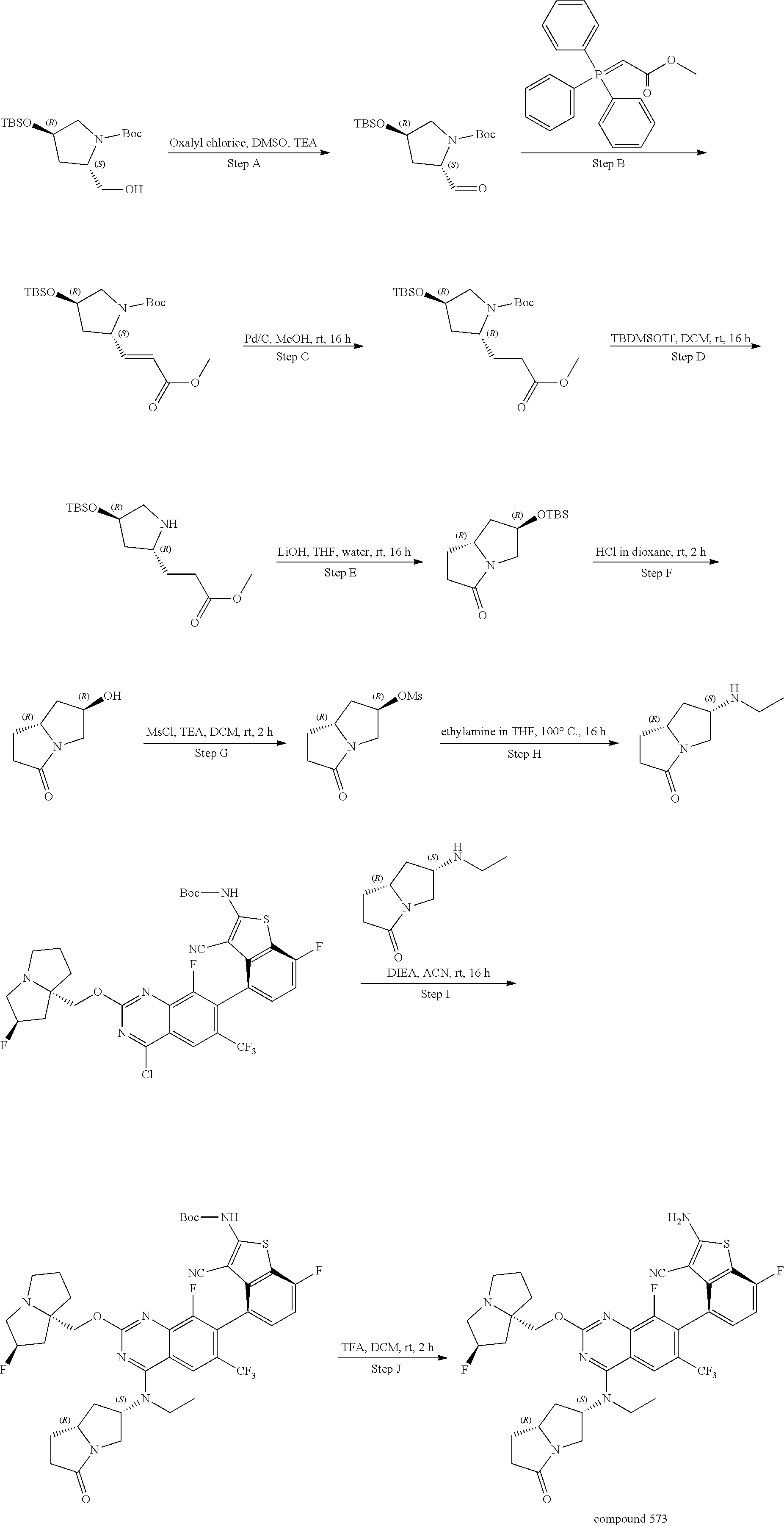

Step A: Preparation of tert-butyl (2S,4R)-4-((tert-butyldimethylsilyl)oxy)-2-formylpyrrolidine-1-carboxylate: To a solution of oxalyl chloride (2.00 eq, 6.9 g, 54.4 mmol) in DCM (70 mL) was added DMSO (4.00 eq. 8.5 g, 108.8 mmol) dropwise at −78° C. under nitrogen atmosphere. The resulting mixture was stirred at −78° C. for 0.5 h under nitrogen atmosphere. To the above mixture was added a solution of tert-butyl (2S,4R)-4-((tert-butyldimethylsilyl)oxy)-2-formylpyrrolidine-1-carboxylate (1.00 eq, 9.0 g, 27.2 mmol) in DCM (100 mL) dropwise at −78° C. under nitrogen atmosphere. The resulting mixture was stirred at −78° C. for 3 h under nitrogen atmosphere. To the above mixture was added TEA (8.00 eq, 22.0 g, 217.6 mmol) dropwise at −78° C. under nitrogen atmosphere. The resulting mixture was stirred at 0° C. for 1 h under nitrogen atmosphere. The reaction was monitored by LCMS. The reaction was quenched by adding water (100 mL) and extracted with DCM (100 mL×3). The combined organic phase was washed with brine (200 mL), dried over anhydrous sodium sulfate and concentrated under vacuum. The residue was purified by silica gel column chromatography eluting with EA in petroleum from 0% to 30% to afford tert-butyl (2S,4R)-4-((tert-butyldimethylsilyl)oxy)-2-formylpyrrolidine-1-carboxylate (6.0 g, 18.23 mmol, 67.05% yield) as a yellow solid. LCMS ESI (+) m/z 330.2 (M+H).

Step B: Preparation of tert-butyl (2S,4R)-4-((tert-butyldimethylsilyl)oxy)-2-((E)-3-methoxy-3-oxoprop-1-en-1-yl) pyrrolidine-1-carboxylate: To a solution of tert-butyl (2S,4R)-4-((tert-butyldimethylsilyl)oxy)-2-formylpyrrolidine-1-carboxylate (1.00 eq. 6.0 g, 18.2 mmol) in THF (100 mL) was added methyl 2-(triphenyl-15-phosphanylidene)acetate (2.00 eq, 12.18 g, 36.4 mmol) in portions at 0° C. under nitrogen atmosphere. The resulting mixture was stirred at 25° C. for 16 h under nitrogen atmosphere. The reaction was monitored by LCMS. The reaction was quenched with water (100 mL) and extracted with ethyl acetate (100 mL×3). The combined organic phase was washed with brine (200 mL), dried over anhydrous sodium sulfate and concentrated under vacuum. The residue was purified by silica gel column chromatography eluting with EA in petroleum from 0% to 30% to afford tert-butyl (2S,4R)-4-((tert-butyldimethylsilyl)oxy)-2-((E)-3-methoxy-3-oxoprop-1-en-1-yl)pyrrolidine-1-carboxylate (5.0 g, 12.95 mmol, 71.15% yield) as a yellow solid. LCMS ESI (+) m/z 386.2 (M+H).

Step C: Preparation of tert-butyl (2R,4R)-4-((tert-butyldimethylsilyl)oxy)-2-(3-methoxy-3-oxopropyl)pyrrolidine-1-carboxylate: To a solution of tert-butyl (2S,4R)-4-((tert-butyldimethylsilyl)oxy)-2-((E)-3-methoxy-3-oxoprop-1-en-1-yl)pyrrolidine-1-carboxylate (1.00 eq. 4.0 g, 10.4 mmol) in MeOH (40 mL) was added Pd/C (1.0 g), the reaction mixture was recharged with nitrogen gas three times and then recharged with hydrogen gas three times. The resulting mixture was stirred at room temperature for 16 h under hydrogen atmosphere. The reaction was monitored by LCMS. The resulting mixture was filtered and the filtrate was concentrated under vacuum to afford crude tert-butyl (2R,4R)-4-((tert-butyldimethylsilyl)oxy)-2-(3-methoxy-3-oxopropyl)pyrrolidine-1-carboxylate (4 g) as a yellow solid, which was used in next step directly without further purification. LCMS ESI (+) m/z 388.2 (M+H).

Step D: Preparation of methyl 3-((2R,4R)-4-((tert-butyldimethylsilyl)oxy)pyrrolidin-2-yl)propanoate: To a solution of tert-butyl (2R,4R)-4-((tert-butyldimethylsilyl)oxy)-2-(3-methoxy-3-oxopropyl) pyrrolidine-1-carboxylate (1.00 eq, 4.5 g, 11.6 mmol) in DCM (50 mL) was added TBDMSOTf (1.50 eq, 3.9 g, 17.4 mmol) and 2,6-lutidine (2.00 eq, 3.1 g, 23.2 mmol) at 0° C. under nitrogen atmosphere. The resulting mixture was stirred at 25° C. for 16 h under nitrogen atmosphere. The reaction was monitored by LCMS. The resulting mixture was diluted with water and extracted with DCM (20 mL×4). The combined organic phase was washed with brine, dried over sodium sulfate, filtered and concentrated under vacuum. The residue was purified by silica gel column chromatography eluting with MeOH in DCM from 1% to 3% to afford methyl 3-((2R,4R)-4-((tert-butyldimethylsilyl)oxy)pyrrolidin-2-yl)propanoate (2.0 g, 6.94 mmol, 59.86% yield) as a yellow solid. LCMS ESI (+) m/z 288.1 (M+H).

Step E: Preparation of (6R,7aR)-6-((tert-butyldimethylsilyl)oxy)hexahydro-3H-pyrrolizin-3-one: To a solution of methyl 3-((2R,4R)-4-((tert-butyldimethylsilyl)oxy)pyrrolidin-2-yl)propanoate (1.00 eq, 2.0 g, 6.9 mmol) in THF (15 mL) and water (5 mL) was added LiOH·H₂O (2.00 eq, 583.3 mg, 13.8 mmol). The resulting mixture was stirred at 25° C. for 16 h. The reaction was monitored by LCMS. The resulting mixture was diluted with water and extracted with DCM (20 mL×4). The combined organic phase was washed with brine, dried over sodium sulfate, filtered and concentrated under vacuum. The residue was purified by silica gel column chromatography eluting with MeOH in DCM from 1% to 5% to afford (6R,7aR)-6-((tert-butyldimethylsilyl)oxy)hexahydro-3H-pyrrolizin-3-one (1.0 g, 3.91 mmol, 56.67% yield) as a yellow solid. LCMS ESI (+) m/z 256.3 (M+H).

Step F: Preparation of (6R,7aR)-6-hydroxyhexahydro-3H-pyrrolizin-3-one: A solution of (6R,7aR)-6-((tert-butyldimethylsilyl)oxy)hexahydro-3H-pyrrolizin-3-one (1.00 eq, 1.0 g, 3.91 mmol) in 4M hydrochloric acid of dioxane (10 mL) was stirred at 25 T for 2 h under nitrogen atmosphere. The reaction was monitored by LCMS. The resulting mixture was concentrated under vacuum to afford crude (6R,7aR)-6-hydroxyhexahydro-3H-pyrrolizin-3-one (1.0 g) as a yellow solid, which was used in next step directly without further purification. LCMS ESI (+) m/z 142.2 (M+H).

Step G: Preparation of (2R,7aR)-5-oxohexahydro-1H-pyrrolizin-2-yl methanesulfonate: To a solution of (6R,7aR)-6-hydroxyhexahydro-3H-pyrrolizin-3-one (1.00 eq, 1.0 g, 7.1 mmol) in DCM (10 mL) was added TEA (3.00 eq, 2.1 g, 21.3 mmol). Then MsCl (1.20 eq, 971.3 mg, 8.52 mmol) was added to the above mixture with dropwise at 0° C. under nitrogen atmosphere. The resulting mixture was stirred at 25° C. for 2 h under nitrogen atmosphere. The reaction was monitored by LCMS. The resulting mixture was diluted with water and extracted with DCM (20 mL×3). The combined organic phase was washed with brine, dried over sodium sulfate, filtered and concentrated under vacuum to afford crude (2R,7aR)-5-oxohexahydro-1H-pyrrolizin-2-yl methanesulfonate (1.0 g) as a yellow oil, which was used in next step directly without further purification. LCMS ESI (+) m/z 220.3 (M+H).

Step H: Preparation of (6S,7aR)-6-(ethylamino)hexahydro-3H-pyrrolizin-3-one: A solution of (2R,7aR)-5-oxohexahydro-1H-pyrrolizin-2-yl methanesulfonate (1.00 eq, 1.0 g, 4.5 mmol) in ethylamine (10 mL, 10% ethylamine in HF) was stirred at 100° C. for 16 h. The reaction was monitored by LCMS. The resulting mixture was concentrated under vacuum. The residue was purified by silica gel column chromatography eluting with MeOH in DCM from 2% to 10% to afford (6S,7aR)-6-(ethylamino)hexahydro-3H-pyrrolizin-3-one (150 mg, 0.9 mmol, 20.0% yield) as a yellow solid. LCMS ESI (+) m/z 169.2 (M+H).

Step I: Preparation of tert-butyl (3-cyano-4-((S)-4-(ethyl((2S,7aR)-5-oxohexahydro-1H-pyrrolizin-2-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophen-2-yl)carbamate: To a solution of tert-butyl (4-((S)-4-chloro-8-fluoro-2-(((2R,7aS)-2-fluorotetrahydro-1H-pyrrolizin-7a(5H)-yl)methoxy)-6-(trifluoromethyl)quinazolin-7-yl)-3-cyano-7-fluorobenzo[b]thiophen-2-yl)carbamate (1.00 eq, 80 mg, 0.115 mmol) in acetonitrile (3 mL) was added (6S,7aR)-6-(ethylamino)hexahydro-3H-pyrrolizin-3-one (2.00 eq, 38.6 mg, 0.230 mmol) and DIEA (5.00 eq, 74.2 mg, 0.575 mmol) at 25° C. under nitrogen atmosphere. The resulting mixture was stirred at 25° C. for 16 h under nitrogen atmosphere. The reaction was monitored by LCMS. The resulting mixture was diluted with water and extracted with ethyl acetate (5 mL×3). The combined organic phase was washed with brin, dried over sodium sulfate, filtered and concentrated under vacuum. The residue was purified by prep-ILC (DCM:MeOH=15:1) to afford tert-butyl (3-cyano-4-((S)-4-(ethyl((2S,7aR)-5-oxohexahydro-1H-pyrrolizin-2-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophen-2-yl)carbamate (50 mg, 0.060 mmol, 52.44% yield) as a yellow solid. LCMS ESI (+) m/z 830.2 (M+H).

Step J: Preparation of 2-amino-4-((S)-4-(ethyl((2S,7aR)-5-oxohexahydro-1H-pyrrolizin-2-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile: To a solution of tert-butyl (3-cyano-4-((S)-4-(ethyl((2S,7aR)-5-oxohexahydro-1H-pyrrolizin-2-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophen-2-yl)carbamate (1.00 eq, 50 mg, 0.060 mmol) in DCM (3 mL) was added TFA (1.0 mL) at 0° C. under nitrogen atmosphere. The resulting mixture was stirred at 25° C. for 2 h under nitrogen atmosphere. The reaction was monitored by LCMS. The reaction was quenched with saturated NaHCO₃ aqueous solution and extracted with DCM (5 mL×3). The combined organic phase was washed with brine, dried over sodium sulfate, filtered and concentrated under vacuum. The residue was purified by prep-TLC (DCM:MeOH=10:1) to afford 2-amino-4-((S)-4-(ethyl((2S,7aR)-5-oxohexahydro-1H-pyrrolizin-2-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile (28.5 mg, 0.039 mmol, 65.16% yield) as a white solid. LCMS ESI (+) m/z 730.1 (M+H). ¹H NMR (400 MHz, CD₃OD) δ 8.19 (s, 1H, 7.22-7.20 (m, 1H, 7.03-6.98 (m, 1H, 5.36 (s, 0.5H), 5.23 (s, 0.5H), 5.14-5.12 (m, 1H, 4.26-4.11 (m, 4H), 3.87-3.79 (m, 3H), 3.56-3.53 (m, 1H), 3.23-3.19 (m, 2H), 3.01 (s, 1H), 2.75-2.66 (m, 3H), 2.41-2.12 (m, 5H), 1.99-1.88 (m, 4H), 1.49-1.46 (m, 3H).

### Synthetic Example 29: 5-((S)-7-(2-amino-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)-N,N,3-trimethyl-4,5,6,7-tetrahydropyrazolo[1,5-a]pyrazine-2-carboxamide (Compound 590)

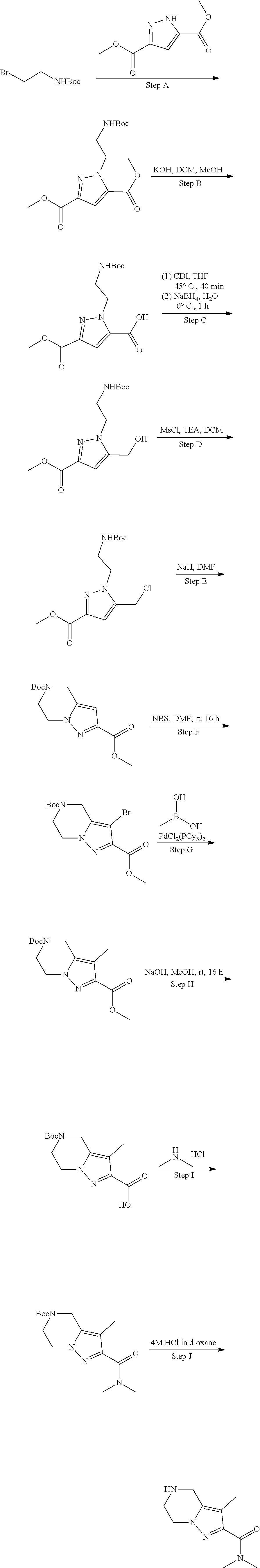

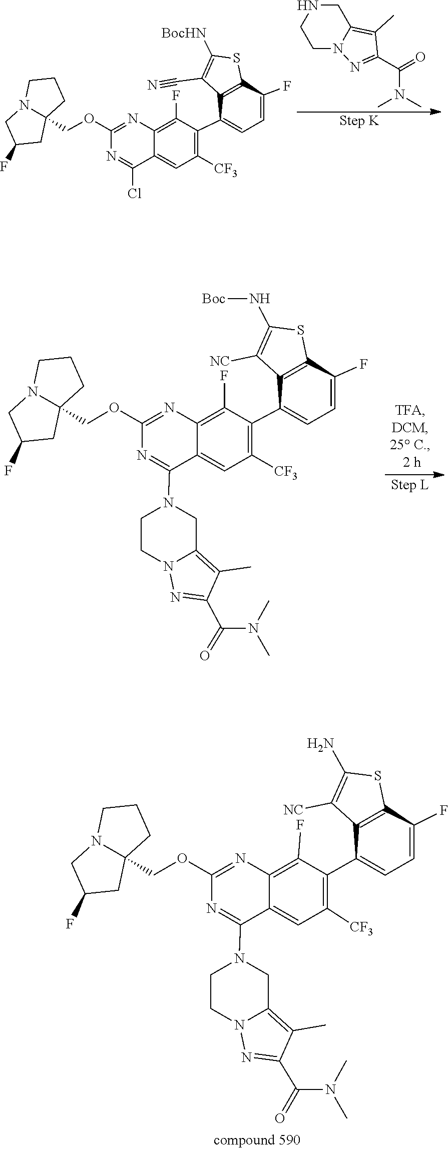

Step A: Preparation of dimethyl 1-(2-((tert-butoxycarbonyl)amino)ethyl)-1H-pyrazole-3,5-dicarboxylate: To a mixture of tert-butyl N-(2-bromoethyl)carbamate (1.00 eq, 60.00 g, 267.6 mmol) and dimethyl 1H-pyrazole-3,5-dicarboxylate (0.770 eq, 38.1 g, 206.1 mmol) in DMF (300 mL) was added Potassium carbonate (3.00 eq, 110.7 g, 4 mmol) at 25° C. The resulting mixture was stirred at 40° C. for 12 h under nitrogen atmosphere. The reaction was monitored by TLC (PE:EA=2:1). The reaction was quenched with water (300 mL), and extracted with ethyl acetate (200 mL×3). The combined organic phase was washed with brine (300 mL), dried over sodium sulfate, filtered and concentrated under vacuum. The residue was purified by silica gel column chromatography eluting with EA in petroleum from 0% to 50% to afford dimethyl 1-[2-(tert-butoxycarbonylamino)ethyl]pyrazole-3,5-dicarboxylate (53.5 g, 151.23 mmol, 56.51% yield). LCMS ESI (+) m/z 328.1 (M+H).

Step B: Preparation of 1-(2-((tert-butoxycarbonyl)amino)ethyl)-3-(methoxycarbonyl)-1H-pyrazole-5-carboxylic acid: To a solution of dimethyl 1-(2-(tert-butoxycarbonylamino)ethyl]pyrazole-3,5-dicarboxylate (1.00 eq 11.80, 36.0 mmol) in DCM (35 mL) and methanol (45 mL) was added KOH (1.00 eq, 2.02 g, 36.0 mmol) at 0° C. The resulting solution was stirred at 25° C. for 12 h. The reaction was monitored by LCMS. The mixture was diluted with water (200 mL) and extracted with DCM (100 mL×3) aqueous phase was adjusted to pH=1 with HCl (1 mol/L) and evaporated in vacuo. When the residue was dried by lyophilization to afford 2-[2-(tert-butoxycarbonylamino)ethyl]-5-methoxycarbonyl-pyrazole-3-carboxylic acid (6.30 g, 20.0 mmol, 55.46% yield). LCMS ESI (+) m/z 314.1 (M+H).

Step C: Preparation of methyl 1-(2-((tert-butoxycarbonyl)amino)ethyl)-5-(hydroxymethyl)-1H-pyrazole-3-carboxylate: To a solution of 2-[2-(tert-butoxycarbonylamino)ethyl]-5-methoxycarbonyl-pyrazole-3-carboxylic acid (1.00 eq, 41.20 g, 131 mmol) in THF (410 mL) was added N,N-carbonyldiimidazole (1.30 eq, 27.72 g, 171 mmol). The reaction mixture was stirred at 45° C. for 40 min under nitrogen atmosphere. The resulting mixture was cooled to 0° C. Then a solution of sodium borohydride (4.00 eq, 19.90 g, 526 mmol) in water (205 mL) was added to above mixture with dropwise at 0° C. under nitrogen atmosphere. The resulting mixture was stirred at 0° C. for 1 h under nitrogen atmosphere. The reaction was monitored by LCMS. The resulting mixture was quenched with water (1 L) and extracted with ethyl acetate (500 mL×3). The combined organic phase was washed with water (1 L) and brine (1 L), dried over sodium sulfate, filtered and concentrated under vacuum. The residue was purified by silica gel column chromatography eluting with MeOH in DCM from 0% to 2% to afford methyl 1-[2-(tert-butoxycarbonylamino)ethyl]-5-(hydroxymethyl)pyrazole-3-carboxylate (32.00 g, 105 mmol, 79.51% yield). LCMS ESI (+) m/z 300.1 (M+H).

Step D: Preparation of methyl 1-(2-((tert-butoxycarbonyl)amino)ethyl)-5-(chloromethyl)-1H-pyrazole-3-carboxylate: To a solution of methyl 1-[2-(tert-butoxycarbonylamino)ethyl]-5-(hydroxymethyl)pyrazole-3-carboxylate (1.00 eq, 26.00 g, 86.9 mmol) in DCM (300 mL) was added TEA (2.00 eq, 17.55 g, 174 mmol). The resulting mixture was cooled to 0° C. and then methanesulfonyl chloride (2.00 eq, 1.1 g, 9.50 mmol) was added to above mixture with dropwise at 0° C. under nitrogen atmosphere. The resulting mixture was stirred at 25° C. for 2 h under nitrogen atmosphere. The reaction was monitored by LCMS. The reaction was quenched with water and extracted with DCM (30 mL×3). The combined organic phase was washed with brine, dried over sodium sulfate, filtered and concentrated under vacuum. The residue was purified by silica gel column chromatography eluting with ethyl acetate in petroleum from 0% to 33% to afford methyl 1-[2-(tert-butoxycarbonylamino)ethyl]-5-(chloromethyl)pyrazole-3-carboxylate (26.00 g, 73.6 mmol, 84.77% yield). LCMS ESI (+) m/z 318.1 (M+H).

Step E: Preparation of 5-(tert-butyl) 2-methyl 6,7-dihydropyrazolo[1,5-a]pyrazine-2,5(4H)-dicarboxylate: To a solution of methyl 1-[2-(tert-butoxycarbonylamino)ethyl]-5-(chloromethyl)pyrazole-3-carboxylate (1.00 eq, 35.00 g, 110 mmol) in DMF (350 mL) was added NaH (2.20 eq, 5.82 g, 242 mmol) at 0° C. under nitrogen atmosphere. The resulting mixture was stirred at 25° C. for 16 h under nitrogen atmosphere. The reaction was monitored by LCMS. The reaction was quenched with water and extracted with ethyl acetate (30 mL×3). The combined organic phase was washed with brine, dried over sodium sulfate, filtered and concentrated under vacuum to afford 5-(tert-butyl) 2-methyl 6,7-dihydropyrazolo[1,5-a]pyrazine-2,5(4H)-dicarboxylate (5.90 g, 18.5 mmol, 16.84% yield), which was used in next step directly without further purification. LCMS ESI (+) m/z 282.1 (M+H).

Step F: Preparation of 5-(tert-butyl) 2-methyl 3-bromo-6,7-dihydropyrazolo[1,5-a]pyrazine-2,5(4H)-dicarboxylate: To a solution of 5-(tert-butyl) 2-methyl 6,7-dihydropyrazolo[1,5-a]pyrazine-2,5(4H)-dicarboxylate (1.00 eq, 1.00 g, 3.55 mmol) in DMF (12 mL) was added NBS (1.50 eq, 0.95 g, 5.33 mmol) at 25° C. under nitrogen atmosphere. The resulting mixture was stirred at 25° C. for 16 h under nitrogen atmosphere. The reaction was monitored by LCMS. The reaction was quenched with water and extracted with ethyl acetate (30 mL×3). The combined organic phase was washed with brine, dried over sodium sulfate, filtered and concentrated under vacuum. The residue was purified by silica gel column chromatography eluting with EA in petroleum from 0% to 33% to afford 5-(tert-butyl) 2-methyl 3-bromo-6,7-dihydropyrazolo[1,5-a]pyrazine-2,5(4H)-dicarboxylate (630 mg, 1.55 mmol, 43.67% yield). LCMS ESI (+) m/z 360.0, 362 (M+H).

Step G: Preparation of 5-(tert-butyl) 2-methyl 3-methyl-6,7-dihydropyrazolo[1,5-a]pyrazine-2,5(4H)-dicarboxylate: To a mixture of 5-(tert-butyl) 2-methyl 3-bromo-6,7-dihydropyrazolo[1,5-a]pyrazine-2,5(4H)-dicarboxylate (1.00 eq. 600 mg, 1.67 mmol) and methylboronic acid (2.00 eq, 199 mg, 3.33 mmol) in 1,4-dioxane (10 mL) was added Cs₂CO (3.00 eq, 1628 mg, 5.00 mmol) and PdCl₂(PCy)₂ (0.200 eq, 246 mg, 0.333 mmol) at 25° C. under nitrogen atmosphere. The resulting mixture was stirred at 70° C. for 16 h under nitrogen atmosphere. The reaction was monitored by LCMS. The reaction was quenched with water and extracted with ethyl acetate (30 mL×3). The combined organic phase was washed with brine, dried over sodium sulfate, filtered and concentrated under vacuum. The residue was purified by prep-TLC to afford 5-(tert-butyl) 2-methyl 3-methyl-6,7-dihydropyrazolo[1,5-a]pyrazine-2,5(4H)-dicarboxylate (450 mg, 1.41 mmol, 84.91% yield). LCMS ESI (+) m/z 296.2 (M+H).

Step H: Preparation of 5-(tert-butoxycarbonyl)-3-methyl-4,5,6,7-tetrahydropyrazolo[1,5-a]pyrazine-2-carboxylic acid: To a solution of 5-(tert-butyl) 2-methyl 3-methyl-6,7-dihydropyrazolo[1,5-a]pyrazine-2,5(4H)-dicarboxylate (1.00 eq, 450 mg, 1.52 mmol) in methanol (4 mL) was added 2.5 M NaOH (aq) (4.0 mL) at 25° C. The resulting mixture was stirred at 25° C. for 16 h. The reaction was monitored by LCMS. The resulting mixture was acidified to pH=3 with 1N HCl and extracted with ethyl acetate (30 mL×3). The combined organic phase was washed with brine, dried over sodium sulfate, filtered and concentrated under vacuum to afford 5-tert-butoxycarbonyl-3-methyl-6,7-dihydro-4H-pyrazolo[1,5-a]pyrazine-2-carboxylic acid (380 mg, 1.30 mmol, 85.46% yield), which was used in next step directly without further purification. LCMS ESI (+) m/z 282.1 (M+H).

Step I: Preparation of tert-butyl 2-(dimethylcarbamoyl)-3-methyl-6,7-dihydropyrazolo[1,5-a]pyrazine-5(4H)-carboxylate: To a mixture of 5-tert-butoxycarbonyl-3-methyl-6,7-dihydro-4H-pyrazolo[1,5-a]pyrazine-2-carboxylic acid (1.00 eq, 380 mg, 1.35 mmol) and dimethylamine hydrochloride (1.00 eq, 110 mg, 1.35 mmol) in DCM (5 mL) was added EDCl (1.00 eq, 259 mg, 1.35 mmol), HOBt (1.00 eq, 182.52 mg, 1.35 mmol) and DIEA (3.00 eq, 523 mg, 4.05 mmol) at 25° C. under nitrogen atmosphere. The resulting mixture was stirred at 25° C. for 16 h under nitrogen atmosphere. The reaction was monitored by LCMS. The reaction was quenched with water and extracted with DCM (30 mL×3). The combined organic phase was washed with brine, dried over sodium sulfate, filtered and concentrated under vacuum. The residue was purified by prep-TLC (DCM:MeOH-15:1) to afford tert-butyl 2-(dimethylcarbamoyl)-3-methyl-6,7-dihydro-4H-pyrazolo[1,5-a]pyrazine-5-carboxylate (295 mg, 0.934 mmol, 69.17% yield). LCMS ESI (+) m/z 309.2 (M+H).

Step J: Preparation of N,N,3-trimethyl-4,5,6,7-tetrahydropyrazolo[1,5-a]pyrazine-2-carboxamide: A solution of tert-butyl 2-(dimethylcarbamoyl)-3-methyl-6,7-dihydro-4H-pyrazolo[1,5-a]pyrazine-5-carboxylate (1.00 eq, 295 mg, 0.957 mmol) in 4M HCl of dioxane (5.0 mL) was stirred at 25° C. for 1 h. The reaction was monitored by LCMS. The reaction mixture was concentrated to afford N,N,3-trimethyl-4,5,6,7-tetrahydropyrazolo[1,5-a]pyrazine-2-carboxamide (230 mg, 0.994 mmol), which was used in next step directly without further purification. LCMS ESI (+) m/z 209.1 (M+H).

Step K: Preparation of tert-butyl (3-cyano-4-((S)-4-(2-(dimethylcarbamoyl)-3-methyl-6,7-dihydropyrazolo[1,5-a]pyrazin-5(4H)-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophen-2-yl)carbamate: To a solution of N,N,3-trimethyl-4,5,6,7-tetrahydropyrazolo[1,5-a]pyrazine-2-carboxamide (2.00 eq, 30 mg, 0.143 mmol) in acetonitrile (3 mL) was added tert-butyl N-[4-[4-chloro-8-fluoro-2-[[(2R,8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate (1.00 eq, 50 mg, 0.0716 mmol) and DIEA (5.00 eq, 46 mg, 0.358 mmol) at 25° C. under nitrogen atmosphere. The resulting mixture was stirred at 25° C. for 16 h under nitrogen atmosphere. The reaction was monitored by LCMS. The reaction was quenched with water and extracted with ethyl acetate (30 mL×3). The combined organic phase was washed with brine, dried over sodium sulfate, filtered and concentrated under vacuum. The residue was purified by prep-TLC (DCM:MeOH=15:1) to afford tert-butyl N-[3-cyano-4-[4-[2-(dimethylcarbamoyl)-3-methyl-6,7-dihydro-4H-pyrazolo[1,5-a]pyrazin-5-yl]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophen-2-yl]carbamate (30 mg, 0.0301 mmol, 42.09% yield). LCMS ESI (+) m/z 870.3 (M+H).

Step L: Preparation of 5-((S)-7-(2-amino-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)-N,N,3-trimethyl-4,5,6,7-tetrahydropyrazolo[1,5-a]pyrazine-2-carboxamide: To a solution of tert-butyl N-[3-cyano-4-[4-[2-(dimethylcarbamoyl)-3-methyl-6,7-dihydro-4H-pyrazolo[1,5-a]pyrazin-5-yl]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophen-2-yl]carbamate (1.00 eq, 30 mg, 0.0345 mmol) in DCM (3 mL) was added TFA (389 eq, 1.0 mL, 13.4 mmol) at 0° C. under nitrogen atmosphere. The resulting mixture was stirred at 25° C. for 1 h. The reaction was monitored by LCMS. The reaction was quenched with saturated NaHCO₃ aqueous solution and extracted with DCM (10 mL×3). The combined organic phase was washed with brine, dried over sodium sulfate, filtered and concentrated under vacuum. The residue was purified by prep-TLC (DCM:MeOH=10:1) to afford 5-[7-(2-amino-3-cyano-7-fluoro-benzothiophen-4-yl)-8-fluoro-2-[[(2R,8S)-2-fluoro-1,2,3,5,6,7-hexahydropyrrolizin-8-yl]methoxy]-6-(trifluoromethyl)quinazolin-4-yl]-N,N,3-trimethyl-6,7-dihydro-4H-pyrazolo[1,5-a]pyrazine-2-carboxamide (20 mg, 0.0244 mmol, 70.62% yield) as a white solid. LCMS ESI (+) m/z 770.2 (M+H). ¹H NMR (400 MHz, CD₃OD) δ 8.23 (s, 1H), 7.23 (s, 1H), 7.06-6.95 (m, 1H), 5.36 (s, 0.5H), 5.23 (s, 0.5H), 5.10 (s, 2H), 4.56-4.22 (m, 6H), 3.29-2.92 (m, 10H), 2.40-1.82 (m, 9H).

### Synthetic Example 30: Preparation of 2-amino-4-((S)-4-(ethyl((2S,8aR)-5-oxooctahydroindolizin-2-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile (Compound 662)

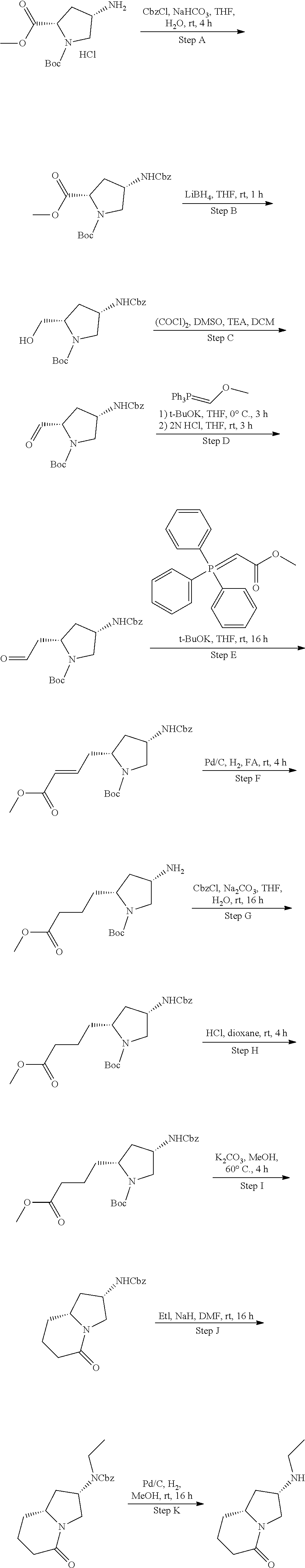

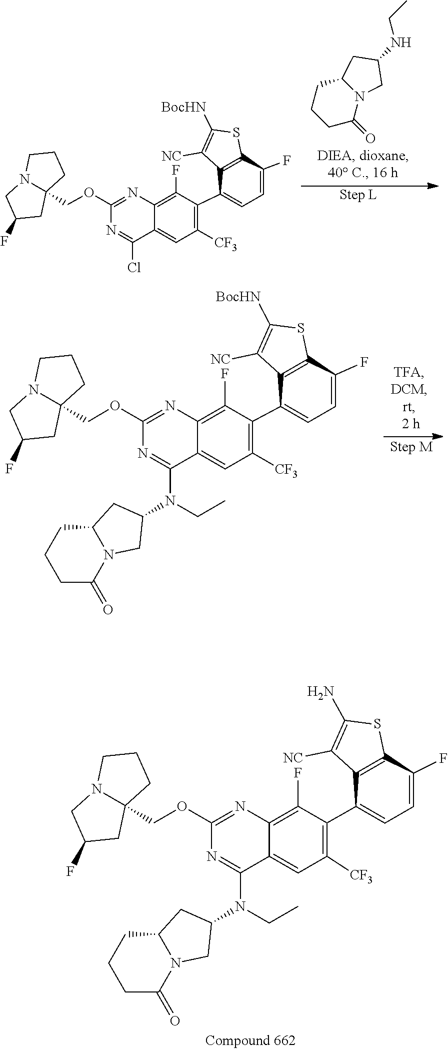

Step A: Preparation of 1-(tert-butyl) 2-methyl (2S,4S)-4-(((benzyloxy)carbonyl)amino)pyrrolidine-1,2-dicarboxylate. To a solution of 1-(tert-butyl) 2-methyl (2S,4S)-4-aminopyrrolidine-1,2-dicarboxylate (1.00 eq, 5.00 g, 0.020 mol) in THF (25 mL) was added water (25 mL) and NaHCO₃ (2.00 eq, 3.44 g, 0.040 mol) at rt under nitrogen atmosphere. Then benzyl carbonochloridate (1.20 eq, 4.09 g, 0.024 mol) was added to above mixture with dropwise at 0° C. under nitrogen atmosphere. The resulting mixture was stirred at rt for 16 h under nitrogen atmosphere. The reaction was monitored by LCMS. The resulting mixture was extracted with ethyl acetate (25 mL×3). The combined organic phase was washed with brine, dried over sodium sulfate, filtered, and concentrated under vacuum. The residue was purified by silica gel column chromatography eluting with ethyl acetate in petroleum from 10% to 35% to afford 1-(tert-butyl) 2-methyl (2S,4S)-4-(((benzyloxy)carbonyl)amino)pyrrolidine-1,2-dicarboxylate (6.51 g, 0.017 mol, 86.11% yield) as a colorless oil. LCMS ESI (+) m/z 323.1 (M+H−56).

Step B: Preparation of tert-butyl (2S,4S)-4-(((benzyloxy)carbonyl)amino)-2-(hydroxymethyl)pyrrolidine-1-carboxylate: To a mixture of lithium borohydride (1.50 eq, 0.55 g, 0.026 mol) in THF (65 mL) was added a solution of 1-(tert-butyl) 2-methyl (2S,4S)-4-(((benzyloxy)carbonyl)amino)pyrrolidine-1,2-dicarboxylate (1.00 eq, 6.51 g, 0.017 mol) in THF (25 mL) dropwise at 0° C. under nitrogen atmosphere. The resulting mixture was stirred at room temperature for 1 h under nitrogen atmosphere. The reaction was monitored by LCMS. The reaction was quenched with ice-water and extracted with ethyl acetate (50 mL×3). The combined organic phase was washed with brine, dried over sodium sulfate, filtered, and concentrated under vacuum. The residue was purified by silica gel column chromatography eluting with EA in petroleum from 10% to 30% to afford tert-butyl (2S,4S)-4-(((benzyloxy)carbonyl)amino)-2-(hydroxymethyl)pyrrolidine-1-carboxylate (4.5 g, 0.013 mol, 76.47% yield) as a colorless oil. LCMS ESI (+) m/z 251.1 (M+H−100), 295.1 (M+H−56).

Step C: Preparation of tert-butyl (2S,4S)-4-(((benzyloxy)carbonyl)amino)-2-formylpyrrolidine-1-carboxylate: To a solution of oxalyl chloride (2.00 eq, 2.17 g, 17.1 mmol) in DCM (30 mL) was added DMSO (4.00 eq, 2.68 g, 34.2 mmol) dropwise over 30 min at −78° C. under nitrogen atmosphere. The resulting mixture was stirred for 1 h at −78° C. under nitrogen atmosphere. To the above mixture was added a solution of tert-butyl (2S,4S)-4-(benzyloxycarbonylamino)-2-(hydroxymethyl)pyrrolidine-1-carboxylate (1.00 eq, 3.00 g, 8.56 mmol) in DCM (10 mL) dropwise over 10 min at −78° C. under nitrogen atmosphere. The resulting mixture was stirred for 4 h at −78° C. under nitrogen atmosphere. Then TEA (8.00 eq, 9.5 mL, 68.5 mmol) was added to above mixture dropwise over 30 min at −78° C. under nitrogen atmosphere. The resulting mixture was stirred for additional 1 h at −78° C. under nitrogen atmosphere. The reaction was monitored by TLC. The resulting mixture was quenched with ice-water, extracted with ethyl acetate (50 mL×3). The combined organic phase was washed with brine, dried over sodium sulfate, filtered, and concentrated under vacuum. The residue was purified by silica gel column chromatography eluting with EA in petroleum from 20% to 50% to afford tert-butyl (2S,4S)-4-(benzyloxycarbonylamino)-2-formyl-pyrrolidine-1-carboxylate (2.10 g, 5.73 mmol, 66.89% yield) as a colorless oil. LCMS ESI (+) m/z 248.8 (M+H−100), 292.8 (M+H−56).

Step D: Preparation of tert-butyl (2S,4S)-4-(((benzyloxy)carbonyl)amino)-2-(2-oxoethyl)pyrrolidine-1-carboxylate: To a solution of (methoxymethylene)triphenyl-15-phosphane (2.00 eq, 4.12 g, 12.06 mmol) in THF (50 mL) was added potassium tert-butoxide (2.50 eq, 1.73 g, 15.07 mmol) at 0° C. under nitrogen atmosphere. Then tert-butyl(2S,4S)-4-(benzyloxycarbonylamino)-2-formyl-pyrrolidine-1-carboxylate (1.00 eq, 2.10 g, 5.73 mmol) was added to the above mixture dropwise at 0° C. under nitrogen atmosphere. The resulting mixture was stirred at rt for 3 h under nitrogen atmosphere. The reaction was monitored by LCMS. The reaction was quenched by the addition of water at 0° C., extracted with ethyl acetate (50 mL×3). The combined organic phase was washed with brine, dried over sodium sulfate, filtered, and concentrated under vacuum. Tho residue was dissolved in THF (50 mL) and 2N HCl (30 mL) was added at 0° C. under nitrogen atmosphere. The resulting mixture was stirred at rt for 3 h under nitrogen atmosphere. The reaction was monitored by LCMS. The reaction was quenched with ice-water, extracted with ethyl acetate (50 mL×3). The combined organic phase was washed with brine, dried over sodium sulfate, filtered, and concentrated under vacuum. The residue was purified by silica gel column chromatography eluting with EA in petroleum from 20% to 30% to afford tert-butyl (2S,4S)-4-(((benzyloxy)carbonyl)amino)-2-(2-oxoethyl)pyrrolidine-1-carboxylate (910 mg, 2.51 mmol, 43.80% yield) as a white solid. LCMS ESI (+) m/z 263.1 (M+H−100), 307.1 (M+H−56).

Step E: Preparation of tert-butyl (2R,4S)-4-(((benzyloxy)carbonyl)amino)-2-((E)-4-methoxy-4-oxobut-2-en-1-yl)pyrrolidine-1-carboxylate: To a solution of tert-butyl (2S,4S)-4-(((benzyloxy)carbonyl)amino)-2-(2-oxoethyl)pyrrolidine-1-carboxylate (1.00 eq, 910 mg, 2.51 mmol) in THF (10 mL) was added potassium tert-butoxide (2.50 eq, 702 mg, 6.27 mmol) at 0° C. under nitrogen atmosphere. Then methyl 2-(triphenyl-15-phosphanylidene)acetate (1.50 eq, 1.26 g, 3.76 mmol) was added to the above mixture dropwise at 0° C. under nitrogen atmosphere. The resulting mixture was stirred at rt for 16 h under nitrogen atmosphere. The reaction was quenched with water and extracted with ethyl acetate (20 mL×3). The combined organic phase was washed with brine, dried over sodium sulfate, filtered and concentrated under vacuum. The residue was purified by silica gel column chromatography eluting with ethyl acetate in petroleum from 10% to 30% to afford tert-butyl (2R,4S)-4-(((benzyloxy)carbonyl)amino)-2-((E)-4-methoxy-4-oxobut-2-en-1-yl)pyrrolidine-1-carboxylate (870 mg, 2.08 mmol, 82.92% yield) as a white solid. LCMS ESI (+) m/z 318.8 (M+H−100), 362.8 (M+H−56).

Step F: Preparation of tert-butyl (2R,4S)-4-amino-2-(4-methoxy-4-oxobutyl)pyrrolidine-1-carboxylate: To a solution of tert-butyl (2R,4S)-4-(((benzyloxy)carbonyl)amino)-2-((E)-4-methoxy-4-oxobut-2-en-1-yl)pyrrolidine-1-carboxylate (1.00 eq, 870 mg, 2.08 mmol) in ethyl acetate (10 mL) was added Pd/C (200 mg). The resulting mixture stirred at 25° C. for 4 h under hydrogen atmosphere. The reaction was monitored by LCMS. The resulting mixture was filtered and the filtrate was concentrated under vacuo to afford tert-butyl (2R,4S)-4-amino-2-(4-methoxy-4-oxobutyl)pyrrolidine-1-carboxylate (500 mg, 1.75 mmol, 84.05% yield) as a colorless oil, which was used in the next step directly without further purification. LCMS ESI (+) m/z 186.8 (M+H−100).

Step G: Preparation of tert-butyl (2R,4S)-4-(((benzyloxy)carbonyl)amino)-2-(4-methoxy-4-oxobutyl)pyrrolidine-1-carboxylate: To a solution of tert-butyl (2R,4S)-4-amino-2-(4-methoxy-4-oxobutyl)pyrrolidine-1-carboxylate (1.00 eq, 500 mg, 1.75 mmol) in THF (5 mL) and water (5 mL) was added NaHCO₃ (2.00 eq, 293.7 mg, 3.49 mmol) at rt under nitrogen atmosphere. Then benzyl carbonochloridate (1.20 eq, 358.2 mg, 2.10 mmol) was added to above mixture dropwise at 0° C. under nitrogen atmosphere. The resulting mixture was stirred at rt for 16 h under nitrogen atmosphere. The reaction was monitored by LCMS. The resulting mixture was extracted with ethyl acetate (10 mL×3). The combined organic phase was washed with brine, dried over sodium sulfate, filtered, and concentrated under vacuum. The residue was purified by silica gel column chromatography eluting with EA in petroleum from 10% to 35% to afford tert-butyl (2R,4S)-4-(((benzyloxy)carbonyl)amino)-2-(4-methoxy-4-oxobutyl)pyrrolidine-1-carboxylate (650 mg, 1.55 mmol, 88.43% yield) as a colorless oil. LCMS ESI (+) m/z 320.9 (M+H−100).

Step H: Preparation of methyl 4-((2R,4S)-4-(((benzyloxy)carbonyl)amino)pyrrolidin-2-yl)butanoate: To a solution of butyl (2R,4S)-4-(((benzyloxy)carbonyl)amino)-2-(4-methoxy-4-oxobutyl)pyrrolidine-1-carboxylate (1.00 eq, 650 mg, 1.55 mmol) in 1,4-dioxane (5 mL) was added 4M HCl in dioxane (5 mL) at 25° C. under nitrogen atmosphere. The resulting mixture was stirred at 25° C. for 4 h under nitrogen atmosphere. The reaction was monitored by LCMS. The solvent was removed under vacuum to afford methyl 4-((2R,4S)-4-(((benzyloxy)carbonyl)amino)pyrrolidin-2-yl)butanoate (590 mg, 1.84 mmol, crude) as a colorless oil, which was used in the next step directly without further purification. LCMS ESI (+) m/z 321.1 (M+H).

Step I: Preparation of benzyl ((2S,8aR)-5-oxooctahydroindolizin-2-yl)carbamate: To a solution of methyl 4-((2R,4S)-4-(((benzyloxy)carbonyl)amino)pyrrolidin-2-yl)butanoate (1.00 eq, 590 mg, 1.84 mmol, crude) in MeOH (8 mL) was added K₂CO₃ (1.50 eq, 380.88 mg, 2.76 mmol) at 25° C. under nitrogen atmosphere. The resulting mixture was stirred at 60° C. for 4 h under nitrogen atmosphere. The reaction was monitored by LCMS. The reaction was quenched with water and extracted with ethyl acetate (50 mL×3). The combined organic phase was washed with brine, dried over sodium sulfate, filtered, and concentrated under vacuum. The residue was purified by silica gel column chromatography eluting with EA in petroleum from 20% to 50% to afford benzyl ((2S,8aR)-5-oxooctahydroindolizin-2-yl)carbamate (300 mg, 1.04 mmol, 56.52% yield) as a white solid. LCMS ESI (+) m/z 288.9 (M+H).

Step J: Preparation of benzyl ethyl((2S,8aR)-5-oxooctahydroindolizin-2-yl)carbamate: To the solvent of DMF (10 mL) was added NaH (1.50 eq, 98 mg, 1.47 mmol) at 0° C. under nitrogen atmosphere. Then a solution of benzyl N-[(2S,8aR)-5-oxo-2,3,6,7,8,8a-hexahydro-1H-indolizin-2-yl]carbamate (1.00 eq, 282 mg, 0.978 mmol) in DMF (1 mL) was added to above mixture with dropwise at 0° C. under nitrogen atmosphere. The resulting mixture was stirred at 25° C. for 1 h under nitrogen atmosphere. Then C₂H₅I (2.00 eq, 305 mg, 1.96 mmol) in DMF (1 mL) was added to above mixture dropwise at 0° C. under nitrogen atmosphere. The resulting mixture was stirred at 25° C. for 16 h under nitrogen atmosphere. The reaction was monitored by LCMS. The resulting mixture was quenched with ice-water and extracted with ethyl acetate (20 mL×4). The combined organic phase was washed with brine, dried over sodium sulfate, filtered and concentrated under vacuum. The residue was purified by silica gel column chromatography eluting with MeOH in DCM from 2% to 10% to afford benzyl ethyl((2S,8aR)-5-oxooctahydroindolizin-2-yl)carbamate (240 mg, 0.721 mmol, 73.69% yield) as a brown oil. LCMS ESI (+) m/z 317.2 (M+H).

Step K: Preparation of (2S,8aR)-2-(ethylamino)hexahydroindolizine-5(1H)-one: To a solution of benzyl N-[(2S,8aR)-5-oxo-2,3,6,7,8,8a-hexahydro-1H-indolizin-2-yl]-N-ethyl-carbamate (1.00 eq, 100 mg, 0.316 mmol) in THF (2 mL) was added Pd/C (25 mg) and Pd(OH)₂ (25 mg). The resulting mixture was stirred at 25° C. for 16 h under hydrogen atmosphere. The reaction was monitored by LCMS. The resulting mixture was filtered and the filtrate was concentrated under vacuum to afford (2S,8aR)-2-(ethylamino)hexahydroindolizin-5(1H)-one (60 mg, 0.263 mmol, 83.32% yield) as a colorless oil, which was used in next step directly without further purification. LCMS ESI (+) m/z 183.1 (M+H).

Step L: Preparation of tert-butyl (3-cyano-4-((S)-4-(ethyl((2S,8aR)-5-oxooctahydroindolizin-2-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophen-2-yl)carbamate: To a solution of tert-butyl N-[4-[4-chloro-8-fluoro-2-[[(2R,8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate (1.00 eq, 70 mg, 0.100 mmol) in 1,4-dioxane (1 mL) was added DIPEA (5.00 eq, 0.089 mL, 0.501 mmol) and (2S,8aR)-2-(ethylamino)hexahydroindolizin-5(1H)-one (1.20 eq, 22 mg, 0.120 mmol) at 40° C. under nitrogen atmosphere. The resulting mixture was stirred at 40° C. for 16 h under nitrogen atmosphere. The reaction was monitored by LCMS. The resulting mixture was diluted with water and extracted with ethyl acetate (10 mL×3). The combined organic phase was washed with brine, dried over sodium sulfate, filtered, and concentrated under vacuum. The residue was purified by Prep-TLC (DCM:MeOH=20:1) to afford tert-butyl (3-cyano-4-((S)-4-(ethyl((2S,8aR)-5-oxooctahydroindolizin-2-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophen-2-yl)carbamate (50 mg, 0.0563 mmol, 56.13% yield) as a yellow solid. LCMS ESI (+) m/z 844.3 (M+H).

Step M: Preparation of 2-amino-4-((S)-4-(ethyl((2S,8aR)-5-oxooctahydroindolizin-2-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile: To a solution of tert-butyl (3-cyano-4-((S)-4-(ethyl((2S,8aR)-5-oxooctahydroindolizin-2-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophen-2-yl)carbamate (1.00 eq, 50 mg, 0.0593 mmol) in DCM (2 mL) was added TFA (0.5 mL) at 25° C. The resulting mixture was stirred at 25° C. for 2 h under nitrogen atmosphere. The reaction was monitored by LCMS. The resulting mixture was concentrated under vacuum and the residue was adjusted to pH 8 with saturated sodium bicarbonate aqueous solution, then extracted with ethyl acetate (10 mL×3). The combined organic phase was washed with water and brine, dried over sodium sulfate, filtered, and concentrated under vacuum. The residue was purified by Prep-TLC (DCM:MeOH=12:1) to afford 2-amino-4-((S)-4-(ethyl((2S,8aR)-5-oxooctahydroindolizin-2-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile (27.8 mg, 0.0367 mmol, 63.18% yield) as an off-white solid. LCMS ESI (+) m/z 744.2 (M+H). ¹H NMR (400 MHz, CD₃OD) δ 8.22 (d, 1H), 7.24-7.18 (m, 1H), 7.01 (t, 1H), 5.39-5.34 (m, 0.5H), 5.25-5.20 (m, 0.5H), 5.02-4.92 (m, 1H), 4.36-4.16 (m, 3H), 3.91-3.68 (m, 4H), 3.28-3.17 (m, 3H), 3.04-2.97 (m, 1H), 2.45-1.75 (m, 14H), 1.49-1.42 (m, 3H).

### Synthetic Example 31: Preparation of 2-amino-4-((S)-4-(ethyl((1S,7aS)-3-oxohexahydro-1H-pyrrolizin-1-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile (Compound 697)

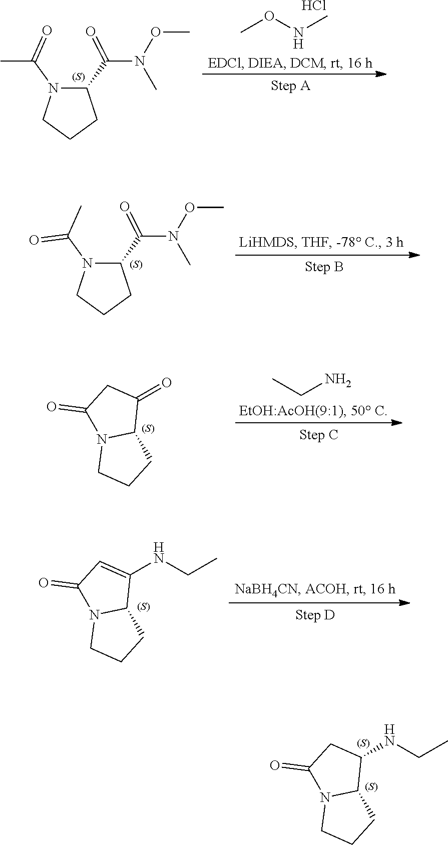

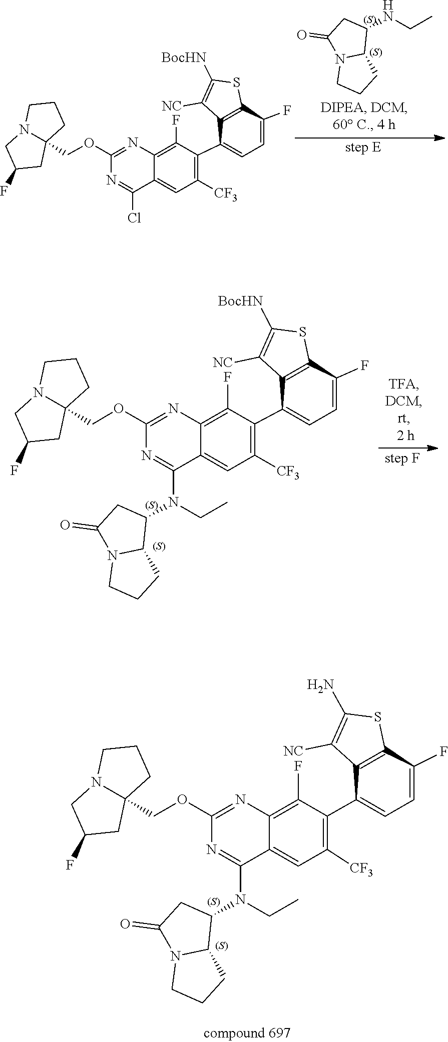

Step A: Preparation of (S)-1-acetyl-N-methoxy-N-methylpyrrolidine-2-carboxamide: To a solution of 1-acetylpyrrolidine-2-carboxylic acid (1.00 eq, 5 g, 31.8 mmol) in DCM (60 mL) was added EDCl (2.00 eq, 12.2 g, 63.6 mmol), HOBT (2.00 eq, 8.6 g, 63.6 mmol) and DIPEA (4.00 eq, 16.41 g, 127 mmol) at room temperature under nitrogen atmosphere. The resulting mixture was stirred at room temperature for 10 minutes under nitrogen atmosphere. Then N,O-dimethylhydroxylamine (1.50 eq, 4.65 g, 47.7 mmol) was added. The resulting mixture was stirred for 2 h at room temperature. The reaction was monitored by LCMS. The reaction was quenched by adding water (20 mL) and extracted with ethyl acetate (20 mL×3). The combined organic phase was washed with water (10 mL×2) and brine (10 mL×2), dried over anhydrous sodium sulfate, and concentrated under vacuum. The residue was purified by silica gel column chromatography (DCM/MeOH=20:1) to afford (S)-1-acetyl-N-methoxy-N-methylpyrrolidine-2-carboxamide (3 g, 12.0 mmol, 37.68% yield) as yellow oil. LCMS ESI (+) m/z 201.23 (M+H).

Step B: Preparation of (S)-tetrahydro-1H-pyrrolizine-1,3(2H)-dione: To a solution of (S)-1-acetyl-N-methoxy-N-methylpyrrolidine-2-carboxamide (1.00 eq, 3 g, 15.0 mmol) in THF (50 mL) was added LiHMDS (2.00 eq, 30 mL, 30.0 mmol) dropwise at −78° C. under nitrogen atmosphere. The resulting mixture was stirred at −30° C. for 3 h under nitrogen atmosphere. The reaction was monitored by LCMS. The resulting mixture was quenched by adding water (10 mL) at 0° C. and extracted with ethyl acetate (10 mL×3).The combined organic phase was washed with brine (10 mL), dried over anhydrous sodium sulfate, concentrated under vacuum to afford (S)-tetrahydro-1H-pyrrolizine-1,3(2H)-dione (2 g, 8.62 mmol, 57.56% yield), which was used in next step directly without further purification. LCMS ESI (+) m/z 140.2 (M+H).

Step C: Preparation of (S)-1-(ethylamino)-5,6,7,7a-tetrahydro-3H-pyrrolizin-3-one. To a solution of(S)-tetrahydro-1H-pyrrolizine-1,3(2H)-dione (1.00 eq, 2 g, 14.3 mmol) in ethanol (9 mL) and AcOH (1 mL) was added a solution of ethanamine in EtOH (5 mL) under nitrogen atmosphere. The resulting mixture was stirred at 50° C. for 3 h under nitrogen atmosphere. The reaction was monitored by LCMS. The resulting mixture was then quenched by adding water (10 mL) and extracted with ethyl acetate (10 mL×3). The combined organic phase was washed with water (10 mL) and brine (10 mL), dried over anhydrous sodium sulfate, and concentrated under vacuum to afford (S)-1-(ethylamino)-5,6,7,7a-tetrahydro-3H-pyrrolizin-3-one, which was used in next step directly without further purification. LCMS ESI (+) m/z 167.11 (M+H).

Step D: Preparation of (1S,7aS)-1-(ethylamino)hexahydro-3H-pyrrolizin-3-one: To a solution of (S)-1-(ethylamino)-5,6,7,7a-tetrahydro-3H-pyrrolizin-3-one (1.00 eq, 2 g, 12.05 mmol) in acetic acid (15 mL) was added NaBH₃CN (2.00 eq, 1.5 g, 9 mmol) at 0° C. under nitrogen atmosphere. The resulting solution was stirred at room temperature for 16 h under nitrogen atmosphere. The reaction was monitored by LCMS. The reaction was then quenched by adding water (10 mL) and extracted with ethyl acetate (10 mL×3). The combined organic phase was dried over anhydrous sodium sulfate and concentrated under vacuum. The residue was purified by silica gel column chromatography eluting with MeOH in DCM from 1% to 5% to afford (1S,7aS)-1-(ethylamino)hexahydro-3H-pyrrolizin-3-one (400 mg, 2.4 mmol, 20% yield). LCMS EST (+) m/z 169.13 (M+H).

Step E: Preparation of tert-butyl (3-cyano-4-((S)-4-(ethyl((1S,7aS)-3-oxohexahydro-1H-pyrrolizin-1-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophen-2-yl)carbamate: To a solution of tert-butyl N-[4-[4-chloro-8-fluoro-2-[[(2R,8S)-2-fluoro-1,2,3,5,6,7-hexahydropyrrolizin-8-yl]methoxyl-6-(trifluoromethyl)quinazolin-7-yl]-3-cyano-7-fluoro-benzothiophen-2-yl]carbamate (1.00 eq, 50 mg, 0.0716 mmol) in 1,4-dioxane (4 mL) was added DIPEA (3.00 eq, 28 mg, 0.215 mmol) and (1S,7aS)-1-(ethylamino)hexahydro-3H-pyrrolizin-3-one (2.00 eq, 24 mg, 0.143 mmol) under nitrogen atmosphere. The resulting mixture was stirred at 60° C. for 4 h under nitrogen atmosphere. The reaction was monitored by LCMS. The resulting mixture was quenched by adding water (5 mL) and extracted with ethyl acetate (10 mL×3). The combined organic phase was washed with water (5 mL) and brine (5 mL), dried over anhydrous sodium sulfate and concentrated under vacuum. The residue was purified by prep-TLC (DCM/MeOH=10:1) to afford tert-butyl (3-cyano-4-((S)-4-(ethyl((1S,7aS)-3-oxohexahydro-1H-pyrrolizin-1-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophen-2-yl)carbamate (20 mg, 0.0193 mmol, 26.92% yield) as a yellow solid. LCMS ESI (+) m/z 830.3 (M+H).

Step F: Preparation of 2-amino-4-((S)-4-(ethyl((1S,7aS)-3-oxohexahydro-1H-pyrrolizin-1-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile: To a solution of tert-butyl (3-cyano-4-((S)-4-(ethyl((1S,7aS)-3-oxohexahydro-1H-pyrrolizin-1-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophen-2-yl)carbamate (1.00 eq, 30 mg, 0.0362 mmol) in DCM (2 mL) was added TFA (0.5 mL) under nitrogen atmosphere. The resulting solution was stirred at room temperature for 2 h under nitrogen atmosphere. The reaction was monitored by LCMS. The resulting mixture was diluted with CH₂Cl₂ (2 mL) and basified to pH 9 with sodium bicarbonate solution and extracted with CH₂Cl₂ (5 mL×3). The combined organic phase was washed with water (3 mL) and brine (2 mL), dried over anhydrous sodium sulfate and concentrated under vacuum. The residue was purified with prep-HPLC to afford 2-amino-4-((S)-4-(ethyl((1S,7aS)-3-oxohexahydro-TH-pyrrolizin-1-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile (5.4 mg, 0.00736 mmol, 20.35% yield) as a white solid. LCMS ESI (+) m/z 730.2 (M+H). ¹H NMR (400 MHz, CD₃OD) δ 8.20 (s, 1H), 7.24-7.21 (m, 1H), 7.04-6.99 (m, 1H), 5.47 (s, 0.5H), 5.34 (s, 1H), 4.50-4.41 (m, 1H), 4.41-4.34 (m, 3H), 3.92-3.87 (m, 2H), 3.61-3.48 (m, 5H), 3.30-3.15 (m, 2H), 2.93-2.86 (m, 1H), 2.87-1.98 (m, 9H), 1.54-1.36 (m, 4H).

### Synthetic Example 32: Preparation of 2-amino-4-((S)-4-(ethyl((3S,5S)-1-((1S,2R)-2-fluorocyclopropane-1-carbonyl)-5-methylpyrrolidin-3-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile (Compound 893)

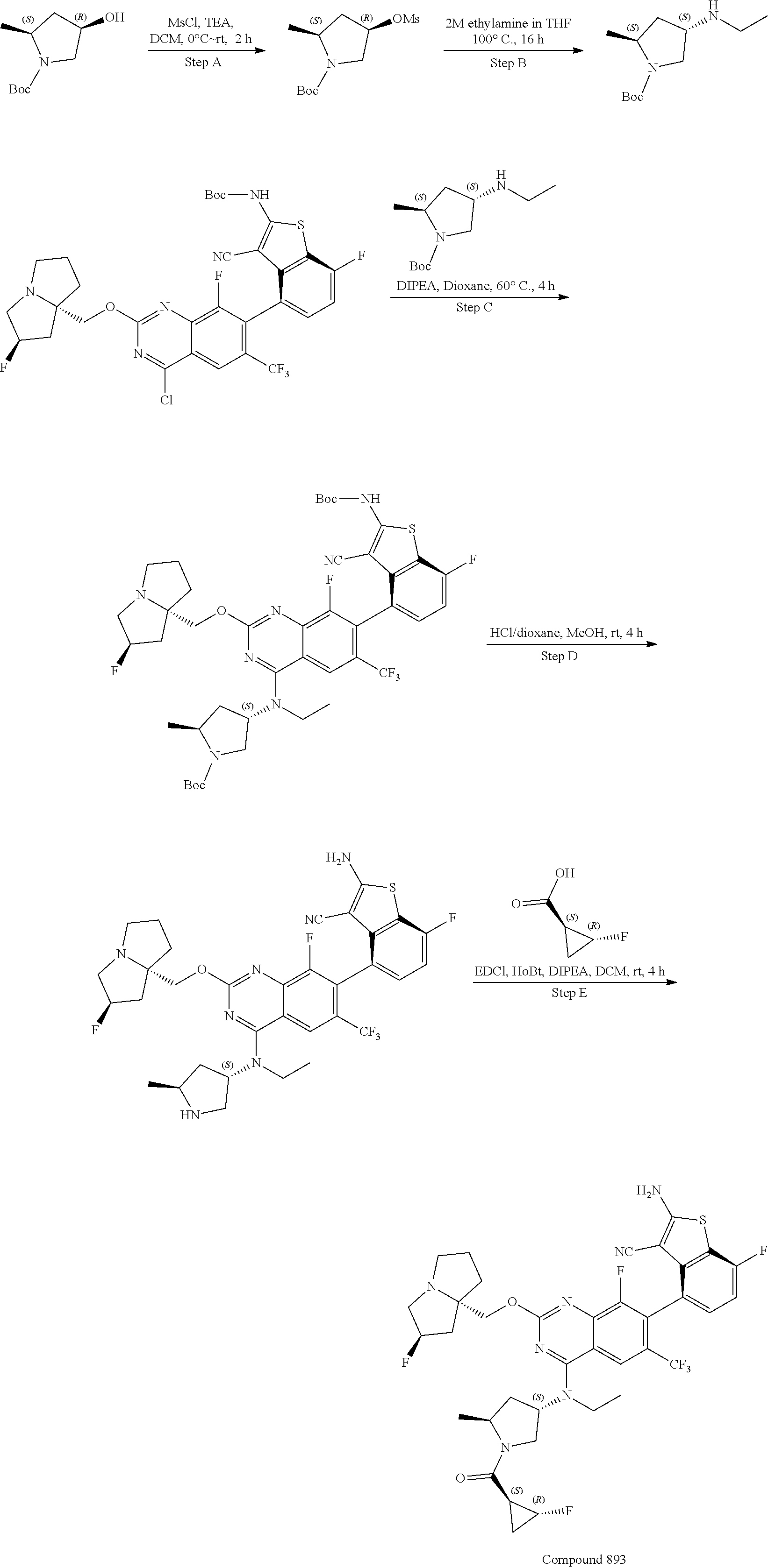

Step A: Preparation of tert-butyl (2S.4R)-2-methyl-4-((methylsulfonyl)oxy)pyrrolidine-1-carboxylate: To a solution of tert-butyl (2S.4R)-4-hydroxy-2-methyl-pyrrolidine-1-carboxylate (1.00 eq. 1.00 g, 4.97 mmol) in DCM (10 mL) was added TEA (3.00 eq. 1506 mg, 14.9 mmol) at room temperature under nitrogen atmosphere. Then MsCl (1.50 eq. 854 mg, 7.45 mmol) was added to above mixture with dropwise at 0° C. under nitrogen atmosphere. The resulting solution was stirred for 2 h at room temperature under nitrogen atmosphere. The reaction was monitored by LCMS. The resulting mixture was quenched with water, extracted with DCM (10 mL×3). The combined organic phase was washed with brine (10 mL), dried over anhydrous sodium sulfate and concentrated under vacuum to afford tert-butyl (2S,4R)-2-methyl-4-methylsulfonyloxy-pyrrolidine-1-carboxylate (1.50 g, 4.56 mmol, 91.86% yield) as a yellow oil, which was used in next step directly without further purification. LCMS (ESI, m/z) 280.2 [M+H].

Step B: Preparation of tert-butyl (2S,4S)-4-(ethylamino)-2-methylpyrrolidine-1-carboxylate: To a solution of tert-butyl (2S.4R)-2-methyl-4-methylsulfonyloxy-pyrrolidine-1-carboxylate (1.00 eq. 1.50 g. 5.37 mmol) in ethanol (10 mL) was added 4M ethylamine in EtOH (20 mL). The resulting mixture was stirred at 100° C. for 16 h. The reaction was monitored by LCMS. The resulting mixture was concentrated under vacuum. The residue was purified by column chromatography (DCM/MeOH=15/1) to afford tert-butyl (2S,4S)-4-(ethylamino)-2-methyl-pyrrolidine-1-carboxylate (329 mg, 1.41 mmol, 26.30% yield) as a yellow oil. LCMS (ESI, m/z): 229.2 [M+H].

Step C: Preparation of tert-butyl (2S,4S)-4(((S)-7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)(ethyl)amino)-2-methylpyrrolidine-1-carboxylate: To a solution of tert-butyl (4-((S)-4-chloro-8-fluoro-2-(((2R,7aS)-2-fluorotetrahydro-1H-pyrrolizin-7a(5H)-yl)methoxy)-6-(trifluoromethyl)quinazolin-7-yl)-3-cyano-7-fluorobenzo[b]thiophen-2-yl)carbamate (1.00 eq. 700 mg, 1.00 mmol) in 1,4-dioxane (10 mL) was added tert-butyl (2S,4S)-4-(ethylamino)-2-methyl-pyrrolidine-1-carboxylate (1.40 eq. 321 mg, 1.40 mmol) and DIPEA (5.00 eq, 0.89 mL, 5.01 mmol). The resulting mixture was stirred at 60° C. for 4 h under nitrogen atmosphere. The reaction was monitored by LCMS. The resulting mixture was quenched by adding water (10 mL) and extracted with ethyl acetate (10 mL×3). The organic phase was washed with water (10 mL) and brine (10 mL), dried over anhydrous sodium sulfate, and concentrated under vacuum. The residue was purified by prep-TLC (DCM/MeOH=10/1) to afford tert-butyl (2S,4S)-4-(((S)-7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)(ethyl)amino)-2-methylpyrrolidine-1-carboxylate (620 mg, 0.620 mmol, 61.83% yield) as a light yellow solid. LCMS (ESI, m/z): 890 [M+H].

Step D: Preparation of 2-amino-4-[4-[ethyl-[(3S,5S)-5-methylpyrrolidin-3-yl]amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile: To a solution of tert-butyl (2S,4S)-4-(((S)-7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)(ethyl)amino)-2-methylpyrimidine-1-carboxylate (1.00 eq. 620 mg, 0.697 mmol) in methanol (4 mL) was added HCl/dioxane (4.0 mL) at room temperature. The resulting solution was stirred at 25° C. for 4 h under nitrogen atmosphere. The reaction was monitored by LCMS. The resulting solution was concentrated under vacuum to afford crude 2-amino-4-[4-[ethyl-[(3S,5S)-5-methylpyrrolidin-3-yl]amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (535 mg, 0.621 mmol, 89.07% yield) as a yellow solid, which was used in next step directly without further purification. LCMS (ESI, m/z): 690 [M+H].

Step E: Preparation of 2-amino-4-((S)-4-(ethyl((3S,5S)-1-((1S,2R)-2-fluorocyclopropane-1-carbonyl)-5-methylpyrrolidin-3-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile: To a solution of 2-amino-4-{4-{ethyl-[(3S,5S)-5-methylpyrrolidin-3-yl]amino]-8-fluoro-2-{[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (1.00 eq, 70.0 mg, 0.101 mmol) in DCM (3 mL) were added EDCl (2.00 eq, 38.0 mg, 0.202 mmol), HOBt (2.00 eq. 27.5 mg, 0.202 mmol), (1S,2R)-2-fluorocyclopropanecarboxylic acid (1.00 eq, 11 mg, 0.101 mmol) and DIPEA (5.00 eq, 0.090 mL, 0.507 mmol) at 0° C. under nitrogen atmosphere. The mixture was stirred at 25° C. for 4 h under nitrogen atmosphere. The reaction was monitored by LCMS. The resulting mixture was washed with water (5 mL) and brine (5 mL). The organic phase was dried over sodium sulfate and concentrated under vacuum. The residue was purified by prep-HPLC to afford 2-amino-4-((S)-4-(ethyl((3S,5S)-1-((1S,2R)-2-fluorocyclopropane-1-carbonyl)-5-methylpyrrolidin-3-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile (35.0 mg, 0.0447 mmol, 44.04% yield) as a white solid. LCMS ESI (+) m/z 776.2 (M+H). ¹H NMR (400 MHz, CD₃OD) δ8.47 (brs, 1H), 8.28 (d, J=9.2 Hz, 1H), 7.23-7.21 (m, 1H), 7.02 (t, J=8.8 Hz, 1H), 5.52 (s, 0.5H), 5.37 (s, 0.5H), 5.31-5.22 (m, 1H), 4.93-4.89 (m, 1H), 4.61-4.37 (m, 3H), 4.29-3.57 (m, 7H), 3.31-3.20 (m, 1H), 2.65-2.01 (m, 9H), 1.47-1.25 (m, 8H).

### Synthetic Example 33: Preparation of 2-amino-7-(fluoro-4-((S)-8-fluoro-24((2R,7aS)-2-fluorotetrahydro-1H-pyrrolizin-7a(5H)-yl)methoxy)-4-(methyl((R)-1-(pyridin-3-yl)ethyl)amino)-6-(trifluoromethyl)quinazolin-7-yl)benzo[b]thiophene-3-carbonitrile (Compound 904)

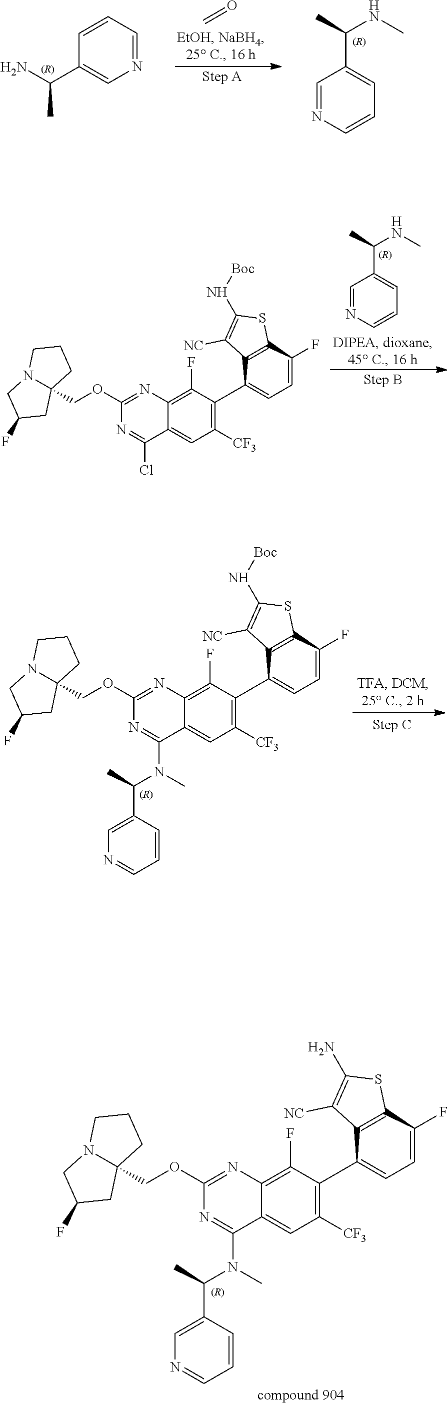

Step A: Preparation of (R)—N-methyl-1-(pyridin-3-yl)ethan-1-amine: To a solution of (R)-1-(pyridin-3-yl)ethan-1-amine (1.00 eq. 201 mg, 1.65 mmol) in ethanol (2 mL) was added formaldehyde (2.00 eq, 267 mg, 3.30 mmol, 37% in water) at 25° C. under nitrogen atmosphere. The resulting mixture was stirred at 25° C. for 16 h under nitrogen atmosphere. Then NaBH₄ (2.00 eq, 125 mg, 3.30 mmol) was added to the above mixture in portions. The resulting mixture was stirred at 25° C. for 2 h under nitrogen atmosphere. The reaction was monitored by LCMS. The resulting mixture was quenched with saturated NH₄Cl aqueous solution. The aqueous solution was extracted with ethyl acetate (10 mL×4). The combined organic phase was washed with brine, dried over anhydrous sodium sulfate, and concentrated under vacuum. The residue was purified by prep-TLC (DCM/MeOH=20:1) to afford (R)—N-methyl-1-(pyridin-3-yl)ethan-1-amine (50 mg, 0.335 mmol, 20.31% yield) as a yellow oil. LCMS ESI (+) m/z 137.2 (M+H).

Step B: Preparation of tert-butyl (3-cyano-7-fluoro-4-((S)-8-fluoro-2-(((2R,7aS)-2-fluorotetrahydro-1H-pyrrolizin-7a(5H)-yl)methoxy)-4-(methyl((R)-1-(pyridin-3-yl)ethyl)amino)-6-(trifluoromethyl)quinazolin-7-yl)benzo[b]thiophen-2-yl)carbamate: To a solution of tert-butyl N-[4-{4-chloro-8-fluoro-2-{[(2R,8S)-2-fluoro-1,2,3,5,6,7-hexahydropyrrolizin-8-yl]methoxy]-6-(trifluoromethyl)quinazolin-7-yl]-3-cyano-7-fluoro-benzothiophen-2-yl]carbamate (1.00 eq, 50 mg, 0.0716 mmol) in 1,4-dioxane (1 mL) was added DIEA (3.00 eq, 0.038 mL, 0.215 mmol) and (R)—N-methyl-1-(pyridin-3-yl)ethan-1-amine (1.20 eq, 12 mg, 0.0860 mmol) at room temperature under nitrogen atmosphere. The resulting mixture was stirred at 45° C. for 16 h under nitrogen atmosphere. The reaction was monitored by LCMS. The resulting mixture was diluted with water and extracted with ethyl acetate (20 mL×3). The combined organic phase was washed with brine, dried over sodium sulfate, filtered, and concentrated under vacuum. The residue was purified by prep-TLC (DCM/MeOH=10/1) to afford tert-butyl (3-cyano-7-fluoro-4-((S)-8-fluoro-2-(((2R,7aS)-2-fluorotetrahydro-1H-pyrrolizin-7a(5H)-yl)methoxy)-4-(methyl((R)-1-(pyridin-3-yl)ethyl)amino)-6-(trifluoromethyl)quinazolin-7-yl)benzo[b]thiophen-2-yl)carbamate (40 mg, 0.0301 mmol, 42.00% yield) as a yellow solid. LCMS ESI (+) m/z 798.2 (M+H).

Step C: Preparation of 2-amino-7-fluoro-4-((S)-8-fluoro-2-(((2R,7aS)-2-fluorotetrahydro-1H-pyrrolizin-7a(5H)-yl)methoxy)-4-(methyl((R)-1-(pyridin-3-yl)ethyl)amino)-6-(trifluoromethyl)quinazolin-7-yl)benzo[b]thiophene-3-carbonitrile: To a solution of tert-butyl (3-cyano-7-fluoro-4-((S)-8-fluoro-2-(((2R,7aS)-2-fluorotetrahydro-1H-pyrrolizin-7a(5H)-yl)methoxy)-4-(methyl((R)-1-(pyridin-3-yl)ethyl)amino)-6-(trifluoromethyl)quinazolin-7-yl)benzo[b]thiophen-2-yl)carbamate (1.00 eq, 40 mg, 0.0501 mmol) in DCM (1 mL) was added TFA (77.7 eq, 0.30 mL, 3.89 mmol) at 25° C. under nitrogen atmosphere. The resulting mixture was stirred at 25° C. for 2 h under nitrogen atmosphere. The reaction was monitored by LCMS. The resulting mixture was concentrated under vacuum. The residue was adjusted to pH 8 with sat. sodium bicarbonate solution and extracted with ethyl acetate (20 mL×3). The combined organic phase was washed with brine, dried over sodium sulfate, filtered, and concentrated under vacuum. The residue was purified by prep-TLC (DCM/MeOH=10/1) to afford 2-amino-7-fluoro-4-((S)-8-fluoro-2-(((2R,7aS)-2-fluorotetrahydro-1H-pyrrolizin-7a(5H)-yl)methoxy)-4-(methyl((R)-1-(pyridin-3-yl)ethyl)amino)-6-(trifluoromethyl)quinazolin-7-yl)benzo[b]thiophene-3-carbonitrile (8.6 mg, 0.0114 mmol, 22.83% yield) as a yellow solid. LCMS ESI (+) m/z 698.2 (M+H). ¹H NMR (400 MHz, CD₃OD) δ 8.63 (s, 1H), 8.50 (d, 1H), 8.31 (s, 1H), 7.94 (d, 1H), 7.47-7.50 (m, 1H), 7.19-7.23 (m, 1H), 7.01 (t, 1H), 6.26-6.33 (m, 1H), 5.35 (s, 0.5H), 5.21 (s, 0.5H), 4.16-4.25 (m, 2H), 3.35-3.48 (m, 2H), 3.16-3.26 (m, 3H), 3.06-3.35 (m, 1H), 2.95-3.02 (m, 1H), 2.13-2.36 (m, 3H), 2.06-2.02 (m, 1H), 1.91-1.99 (m, 2H), 1.84 (d, 3H).

### Synthetic Example 34: Preparation of 2-amino-4-((S)-4-(ethyl((S)-1-(thiazole-5-carbonyl)pyrrolidin-3-yl)amino)-8-fluoro-2-(((2R,7 aS-2-fluorotetrahydro-1H-pyrrolizin-7a(5H)-yl)methoxy)-6-trifluoromethyl)quinazolin-7-yl)-7-fluorobenzo[b]thiophene-3-carbonitrile (Compound 906)

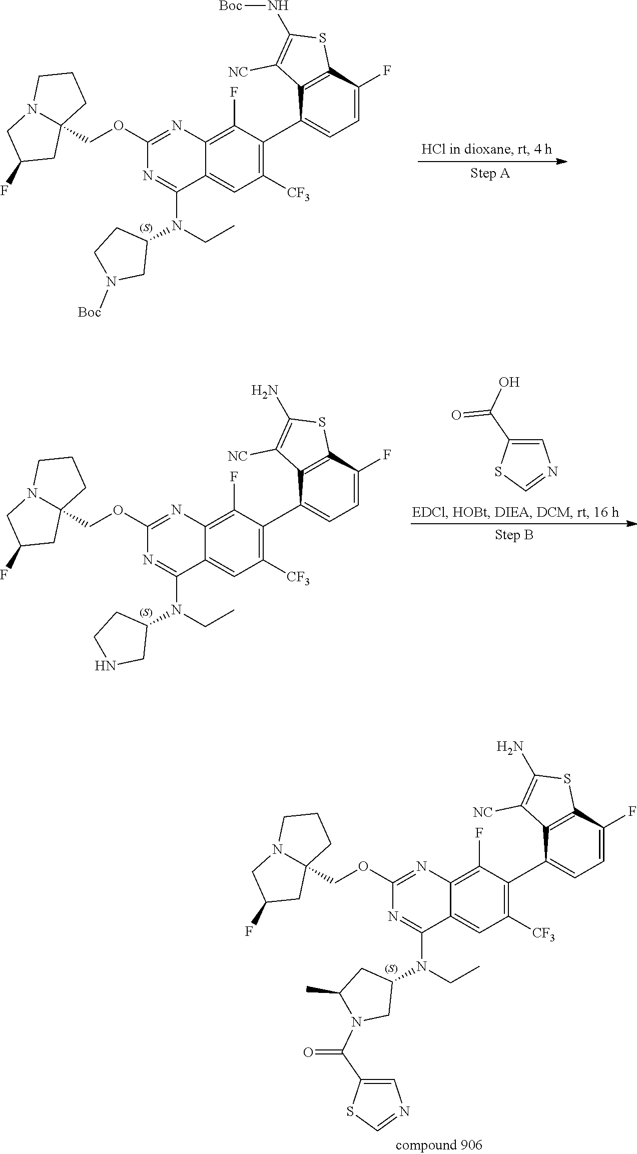

Step A: Preparation of 2-amino-4-((S)-4-(ethyl((S)-pyrrolidin-3-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile: To a solution of tert-butyl (S)-3-(((S)-7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)(ethyl)amino)pyrrolidine-1-carboxylate (1 eq, 60 mg, 0.07 mmol) in MeOH (2 mL) was added HCl/dioxane (2 mL). The resulting solution was stirred at room temperature for 2 h. The reaction was monitored by LCMS. The solvent was evaporated under vacuum to afford crude 2-amino-4-((S)-4-(ethyl((S)-pyrrolidin-3-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile (40 mg, 0.06 mmol, 87% yield) as a yellow solid, which was used in next step directly without further purification. LCMS (ESI, m/z): 676.2 [M+H].

Step B: Preparation of 2-amino-4-((S)-4-(ethyl((S)-1-(thiazole-5-carbonyl)pyrrolidin-3-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile: To a solution of thiazole-5-carboxylic acid (0.700 eq, 5.4 mg, 0.0414 mmol) in DCM (3 mL) were added DIPEA (3.00 eq, 23 mg, 0.18 mmol), EDCl (2.00 eq, 22 mg, 0.12 mmol) and HOBt (2.00 eq, 16 mg, 0.12 mmol) at room temperature under nitrogen atmosphere. The resulting mixture was stirred at room temperature for 10 minutes. Then 2-amino-4-[4-[ethyl-[(3S)-pyrrolidin-3-yl]amino]-8-fluoro-2-[[(2R,8S)-2-fluoro-1,2,3,5,6,7-hexahydropyrrolizin-8-yl]methoxy]-6-(trifluoromethyl)quinazolin-7-yl]-7-fluoro-benzothiophene-3-carbonitrile (1.00 eq, 40 mg, 0.0592 mmol) was added to above mixture at room temperature. The resulting mixture was stirred for 16 h at room temperature. The reaction was monitored by LCMS. The reaction was quenched with water (3 mL) and extracted with ethyl acetate (5 mL×3). The combined organic phase was washed with water (2 mL) and brine (2 mL), dried over anhydrous sodium sulfate, and concentrated under vacuum. The residue was purified by prep-TLC (DCM/MeOH=10/1) to afford 2-amino-4-((S)-4-(ethyl((S)-1-(thiazole-5-carbonyl)pyrrolidin-3-yl)amino)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[b]thiophene-3-carbonitrile (25 mg, 0.0314 mmol, 53.07% yield) as a light yellow solid. LCMS ESI (+) m/z 787.20 (M+H). ¹H NMR (400 MHz, CD₃OD) δ 9.17-9.15 (m, 1H), 8.54-8.42 (m, 1H), 8.38-8.26 (s, 1H), 7.23-7.21 (m, 1H).7.03-6.99 (m, 1H), 5.32 (s, 0.5H), 5.28 (s, 0.5H), 5.19-5.08 (m, 1H), 4.28 (m, 0.5H), 4.28-4.18 (m, 3H), 4.00-3.89 (m, 4H), 3.69 (m, 0.5H), 3.31-3.14 (m, 3H), 2.64 (s, 1H), 2.64-2.22 (m, 2H), 2.13-1.82 (m, 6H), 1.49 (s, 3H).

A compound of present disclosure, such as a compound of a formula included in Table 5, Table 6, Table 7, Table 8, Table 9, or Table 10 may be synthesized according to one of the general routes outlined in Synthetic Examples 1-29 or by various other methods generally known in the art.

Table 5 includes selected compounds of the present disclosure.

TABLE 5
Selected compounds of the present disclosure.

| Cmpd. | | |
|---|---|---|
| No. | Structure | Analytical Data |
| 1 | 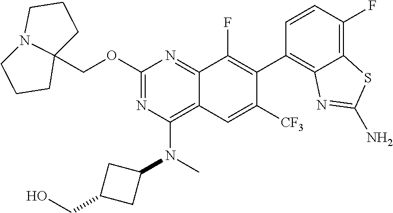 | LCMS m/z [M + 1]: 635.3 1H NMR (400 MHz, CD3OD): δ 8.25 (s, 1H), 7.21 (dd, J = 8.0, 5.2 Hz, 1H), 6.99 (t, J = 8.8 Hz, 1H), 4.98-4.95 (m, 1H). 4.65 (s, 2H), 3.68 (d, J = 6.8 Hz, 4H), 3.30-3.25 (m, 2H), 3.48 (s, 3H), 2.80-2.00 (m, 13H). |
| 1a | 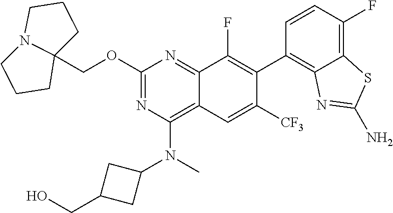 [SMILES: CN(c1nc(OCC23CCCN2CCC3)nc2c(F)c(-c3ccc(F)c4sc(N)nc34)c(C(F)(F)F)cc12)C1CC(CO)C1] | |
| 2 | 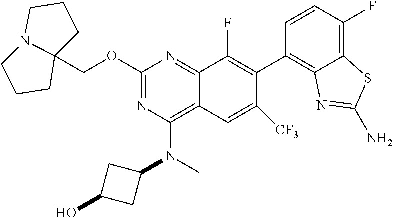 | LCMS m/z [M + 1]: 621.2 1H NMR (400 MHz, CD3OD): δ 8.28 (s, 1H), 7.20 (dd, J = 8.0, 5.6 Hz, 1H), 7.00 (t, J = 8.8 Hz, 1H), 4.66 (s, 2H), 4.38 (t, J = 7.2 Hz, 1H), 4.09 (t, J = 7.2 Hz, 1H), 3.80-3.70 (m, 2H), 3.48 (s, 3H), 3.30-3.25 (m, 2H), 2.90-2.80 (m, 2H), 2.40-2.05 (m, 10H). |
| 3 | 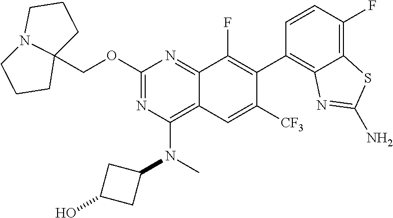 | LCMS m/z [M + 1]: 621.1 1H NMR (400 MHz, CD3OD) δ 8.26 (s, 1H), 7.21 (dd, J = 8.0, 5.6 Hz, 1H), 6.99 (t, J = 8.8 Hz, 1H), 5.20-5.15 (m, 1H). 4.66 (s, 2H), 4.46 (t, J = 6.8 Hz, 1H), 3.80-3.70 (m, 2H), 3.46 (s, 3H), 3.30-3.25 (m, 2H), 2.80-2.70 (m, 2H), 2.50-2.00 (m, 10H). |
| 3a | 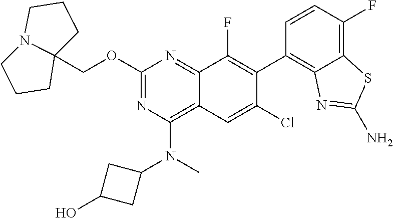 [SMILES: CN(c1nc(OCC23CCCN2CCC3)nc2c(F)c(-c3ccc(F)c4sc(N)nc34)c(Cl)cc12)C1CC(O)C1] | |
| 4 | 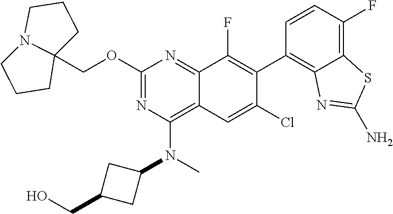 | LCMS m/z [M + 1]: 635.3 1H NMR (400 MHz, CD3OD): δ 8.25 (s, 1H), 7.19 (dd, J = 8.0, 5.6 Hz, 1H), 6.97 (t, J = 8.8 Hz, 1H), 4.80-4.70 (m, 1H), 4.58 (s, 2H), 3.59 (d, J = 4.8 Hz, 2H), 3.55-3.45 (m, 2H), 3.42 (s, 3H), 3.15-3.02 (m, 2H), 2.60-2.45 (m, 2H), 2.40-2.00 (m, 11H). |
| 4a | 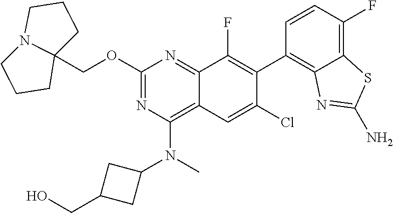 [SMILES: CN(c1nc(OCC23CCCN2CCC3)nc2c(F)c(-c3ccc(F)c4sc(N)nc34)c(Cl)cc12)C1CC(CO)C1] | |
| 5 | 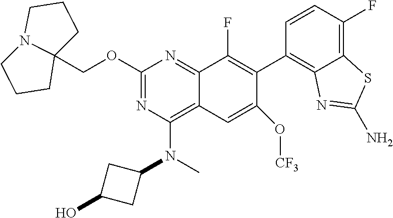 | LCMS m/z [M + 1]: 635.2 1H NMR (400 MHz, CD3OD): δ 7.88 (s, 1H), 7.27 (dd, J = 8.0, 5.6 Hz, 1H), 7.02 (t, J = 8.8 Hz, 1H), 4.67 (s, 2H), 4.40-4.30 (m, 1H), 4.12-4.05 (m, 1H), 3.80-3.70 (m, 2H), 3.44 (s, 3H), 3.30-3.25 (m, 2H), 2.84 (dd, J = 6.8, 4.4 Hz, 2H), 2.50-2.00 (m, 10H). |
| 5a | 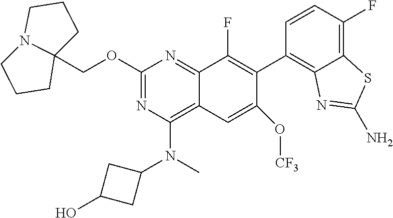 [SMILES: CN(c1nc(OCC23CCCN2CCC3)nc2c(F)c(-c3ccc(F)c4sc(N)nc34)c(OC(F)(F)F)cc12)C1CC(O)C1] | |
| 6 | 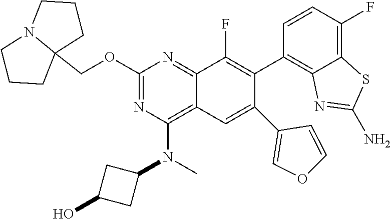 [SMILES: CN(c1nc(OCC23CCCN2CCC3)nc2c(F)c(-c3ccc(F)c4sc(N)nc34)c(-c3ccoc3)cc12)[C@H]1C[C@@H](O)C1] | LCMS m/z [M + 1]: 619.2 1H NMR (400 MHz, CD3OD): δ 8.02 (s, 1H), 7.34 (t, J = 1.6 Hz, 1H), 7.15-7.10 (m, 2H), 6.96 (t, J = 8.8 Hz, 1H), 6.16 (d, J = 0.80 Hz, 1H), 4.71 (s, 2H), 4.55-4.45 (m, 1H), 4.25-4.15 (m, 1H), 3.75-3.65 (m, 2H), 3.55 (s, 3H), 3.30-3.20 (m, 2H), 2.92-2.82 (m, 2H), 2.50-2.00 (m, 10H). |
| 6a | 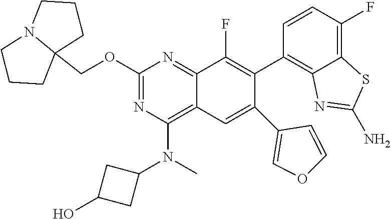 [SMILES: CN(c1nc(OCC23CCCN2CCC3)nc2c(F)c(-c3ccc(F)c4sc(N)nc34)c(-c3ccoc3)cc12)C1CC(O)C1] | |
| 7 | 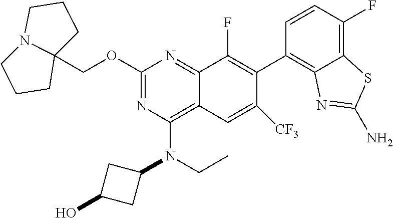 | LCMS m/z [M + 1]: 635.2 1H NMR (400 MHz, CD3OD): δ 8.25 (s, 1H), 7.22 (dd, J = 8.4, 5.6 Hz, 1H), 6.99 (t, J = 8.8 Hz, 1H), 4.66 (s, 2H), 4.40-4.30 (m, 1H), 4.15-4.05 (m, 1H), 4.05-3.95 (m, 2H), 3.75-3.65 (m, 2H), 3.30-3.25 (m, 2H), 2.90-2.80 (m, 2H), 2.50-2.00 (m, 10H), 1.45- 1.35 (m, 3H). |
| 7a | 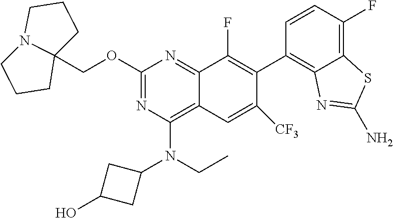 [SMILES: CCN(c1nc(OCC23CCCN2CCC3)nc2c(F)c(-c3ccc(F)c4sc(N)nc34)c(C(F)(F)F)cc12)C1CC(O)C1] | |
| 8 | 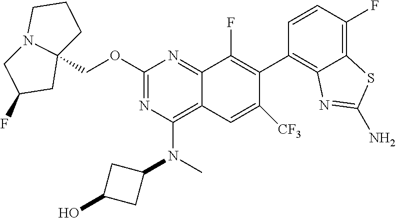 | LCMS m/z [M + 1]: 639.2 1H NMR (400 MHz, CD3OD): δ 8.24 (s, 1H), 7.25- 7.15 (m, 1H), 6.99 (t, J = 8.8 Hz, 1H), 5.44 (d, J = 52.8 Hz, 1H), 4.70-4.40 (m, 2H), 4.40-4.28 (m, 1H), 4.15-4.05 (m, 1H), 3.80-3.50 (m, 2H), 3.44 (s, 3H), 3.30-3.25 (m, 2H), 2.90-2.80 (m, 2H), 2.60-1.95 (m, 8H). |
| 8a | 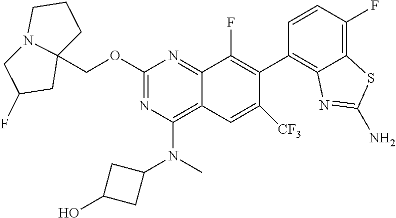 [SMILES: CN(c1nc(OCC23CCCN2CC(F)C3)nc2c(F)c(-c3ccc(F)c4sc(N)nc34)c(C(F)(F)F)cc12)C1CC(O)C1] | |
| 9 | 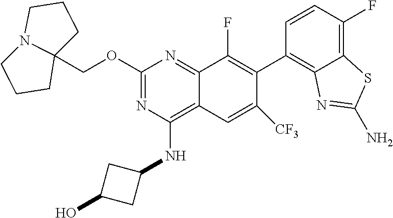 | LCMS m/z [M + 1]: 607.2 1H NMR (400 MHz, CD3OD): δ 8.65 (s, 1H), 7.22 (dd, J = 8.4, 5.6 Hz, 1H), 7.01 (t, J = 8.8 Hz, 1H), 4.69 (s, 2H), 4.40-4.30 (m, 1H), 4.20-4.10 (m, 1H), 3.75-3.65 (m, 2H), 3.30-3.25 (m, 2H), 2.90-2.80 (m, 2H), 2.40-2.00 (m, 10H). |
| 9a | 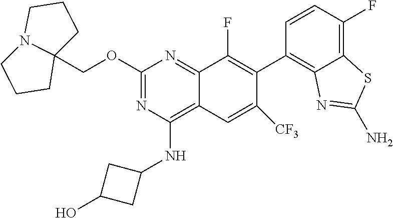 [SMILES: Nc1nc2c(-c3c(C(F)(F)F)cc4c(NC5CC(O)C5)nc(OCC56CCCN5CCC6)nc4c3F)ccc(F)c2s1] | |
| 10 | 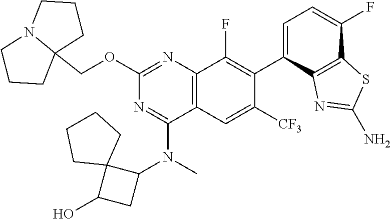 | LCMS m/z [M + 1]: 675.3 1H NMR (400 MHz, CD3OD): δ 8.35 (s, 1H), 7.25- 7.20 (m, 1H), 7.00 (t, J = 8.8 Hz, 1H), 4.80-4.70 (m, 1H), 4.63-4.58 (m, 1H), 4.55-4.46 (m, 1H), 3.90-3.85 (m, 1H), 3.70-3.60 (m, 2H), 3.55 (s, 3H), 3.30-3.25 (m, 2H), 2.75-2.68 (m, 1H), 2.68-2.60 (m, 1H), 2.40- 2.00 (m, 8H), 1.81-1.71 (m, 1H), 1.70-1.20 (m, 7H). |
| 11 | 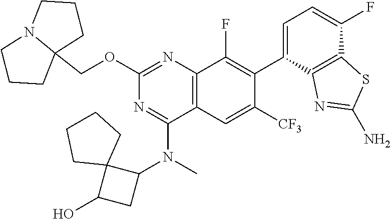 | LCMS m/z [M + 1]: 675.3 1H NMR (400 MHz, CD3OD): δ 8.34 (s, 1H), 7.25- 7.20 (m, 1H), 7.00 (t, J = 8.8 Hz, 1H), 4.80-4.70 (m, 1H), 4.63-4.58 (m, 1H), 4.55-4.46 (m, 1H), 3.90-3.85 (m, 1H), 3.70-3.60 (m, 2H), 3.55 (s, 3H), 3.30-3.25 (m, 2H), 2.75-2.68 (m, 1H), 2.66-2.58 (m, 1H), 2.40- 2.00 (m, 8H), 1.81-1.71 (m, 1H), 1.70-1.20 (m, 7H). |
| 11a | 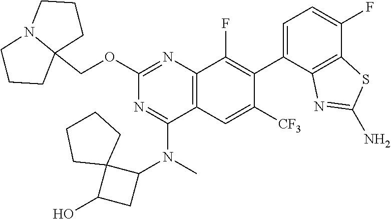 [SMILES: CN(c1nc(OCC23CCCN2CCC3)nc2c(F)c(-c3ccc(F)c4sc(N)nc34)c(C(F)(F)F)cc12)C1CC(O)C12CCCC2] | |
| 12 | 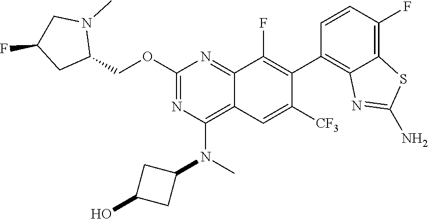 | LCMS m/z [M + 1]: 613.2 1H NMR (400 MHz, CD3OD): δ 8.29 (s, 1H), 7.28- 7.22 (m, 1H), 7.08-7.00 (m, 1H), 5.46 (d, J = 52.4 Hz, 1H), 5.04-4.98 (m, 1H), 4.78-4.72 (m, 1H), 4.50- 4.40 (m, 1H), 4.32-4.22 (m, 1H), 4.15-3.95 (m, 2H), 3.75-3.62 (m, 1H), 3.48 (s, 3H), 3.16 (s, 3H), 2.94- 2.84 (m, 2H), 2.73-2.61 (m, 1H), 2.50-2.35 (m, 1H), 2.35-2.22 (m, 2H). |
| 12a | 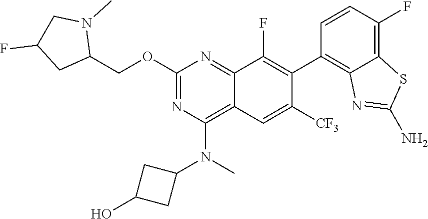 [SMILES: CN1CC(F)CC1COc1nc(N(C)C2CC(O)C2)c2cc(C(F)(F)F)c(-c3ccc(F)c4sc(N)nc34)c(F)c2n1] | |
| 13 | 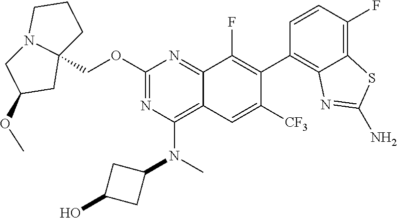 | LCMS m/z [M + 1]: 651.2 1H NMR (400 MHz, CD3OD): δ 8.29 (s, 1H), 7.30- 7.24 (m, 1H), 7.02 (t, J = 8.80 Hz, 1H), 4.67-4.62 (m, 2H), 4.42-4.38 (m, 1H), 4.28 (s, 1H), 4.05-4.12 (m, 1H), 3.83-3.74 (m, 2H), 3.70-3.60 (m, 1H), 3.49 (s, 4H), 3.44-3.38 (m, 4H), 2.91-2.82 (m, 2H), 2.50-2.40 (m, 2H), 2.38-2.25 (m, 5H), 2.18-2.08 (m, 1H). |
| 14 | 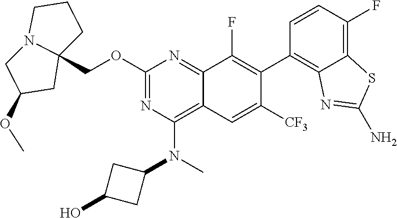 | LCMS m/z [M + 1]: 651.2 1H NMR (400 MHz, CD3OD): δ 8.28 (s, 1H), 7.28- 7.22 (m, 1H), 7.00 (t, J = 8.80 Hz, 1H), 4.71-4.59 (m, 2H), 4.45-4.35 (m, 1H), 4.27 (s, 1H), 4.15-4.05 (m, 1H), 3.85-3.75 (m, 2H), 3.68-3.60 (m 1H), 3.55-3.46 (m, 4H), 3.38 (s, 3H), 2.91-2.82 (m, 2H), 2.50-2.40 (m, 2H), 2.38-2.20 (m, 5H), 2.15-2 .-05 (m, 1H). |
| 14a | 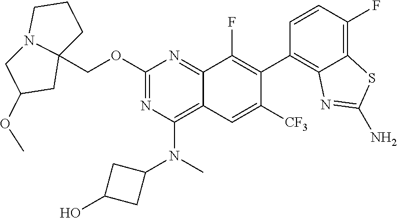 [SMILES: COC1CN2CCCC2(COc2nc(N(C)C3CC(O)C3)c3cc(C(F)(F)F)c(-c4ccc(F)c5sc(N)nc45)c(F)c3n2)C1] | |
| 15 | 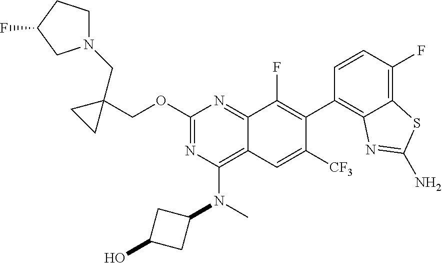 | LCMS m/z [M + 1]: 651.2 1H NMR (400 MHz, CD3OD): δ 8.28 (s, 1H), 7.28- 7.22 (m, 1H), 7.00 (t, J = 8.80 Hz, 1H), 5.55-5.40 (m, 1H), 4.70-4.60 (m, 1H), 4.48-4.37 (m, 2H), 4.20-4.35 (m, 9H), 3.26-3.12 (m, 1H), 2.92-2.84 (m, 2H), 2.50- 2.18 (m, 4H), 1.01-0.82 (m, 4H). |
| 15a | 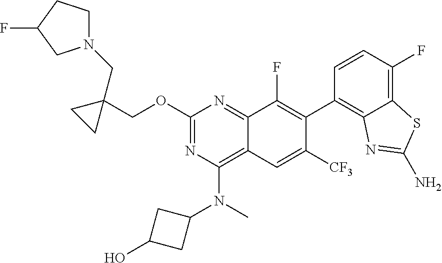 [SMILES: CN(c1nc(OCC2(CN3CCC(F)C3)CC2)nc2c(F)c(-c3ccc(F)c4sc(N)nc34)c(C(F)(F)F)cc12)C1CC(O)C1] | |
| 16 | 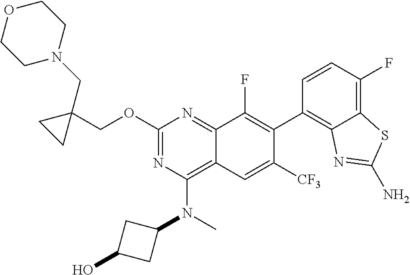 | LCMS m/z [M + 1]: 651.3 1H NMR (400 MHz, CD3OD): δ 8.29 (s, 1H), 7.28- 7.22 (m, 1H), 7.01 (t, J = 8.80 Hz, 1H), 4.78-4.68 (m, 1H), 4.51-4.38 (m, 2H), 4.15-3.60 (m, 7H), 3.50 (s, 3H), 3.45-3.35 (m, 2H), 3.30-3.15 (m, 2H), 2.92-2.84 (m, 2H), 2.38-2.26 (m, 2H), 1.03-0.90 (m, 4H). |
| 16a | 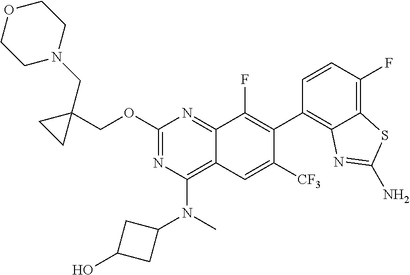 [SMILES: CN(c1nc(OCC2(CN3CCOCC3)CC2)nc2c(F)c(-c3ccc(F)c4sc(N)nc34)c(C(F)(F)F)cc12)C1CC(O)C1] | |
| 17 | 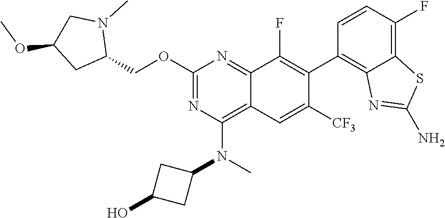 | LCMS m/z [M + 1]: 625.2 1H NMR (400 MHz, CD3OD): δ 8.28 (s, 1H), 7.28- 7.22 (m, 1H), 7.00 (t, J = 8.80 Hz, 1H), 4.75-4.65 (m, 2H), 4.38-4.35 (m, 1H), 4.22-4.05 (m, 3H), 3.95-3.73 (m, 1H), 3.48 (s, 3H), 3.39 (br s, 4H), 3.12 (s, 3H), 2.92-2.84 (m, 2H), 2.60-2.50 (m, 1H), 2.38-2.25 (m, 2H), 2.25-2.10 (m, 1H). |
| 17a | 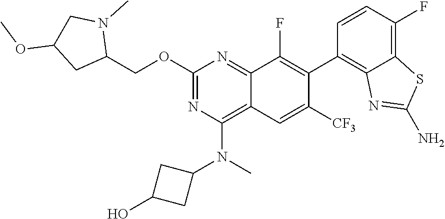 [SMILES: COC1CC(COc2nc(N(C)C3CC(O)C3)c3cc(C(F)(F)F)c(-c4ccc(F)c5sc(N)nc45)c(F)c3n2)N(C)C1] | |
| 18 | 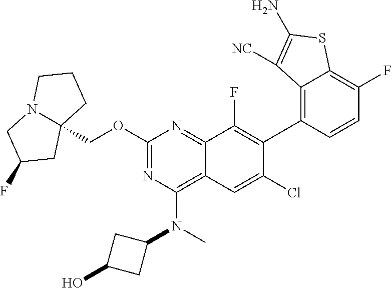 [SMILES: CN(c1nc(OC[C@@]23CCCN2C[C@H](F)C3)nc2c(F)c(-c3ccc(F)c4sc(N)c(C#N)c34)c(Cl)cc12)[C@H]1C[C@@H](O)C1] | LCMS m/z [M + 1]: 663.2 1H NMR (400 MHz, CD3OD): δ 8.27 (s, 1H), 7.28- 7.22 (m, 1H), 7.02 (t, J = 8.80 Hz, 1H), 5.57 (d, J = 51.6 Hz, 1H), 4.80-4.60 (m, 3H), 4.40-4.30 (m, 1H), 4.15-4.00 (m, 2H), 4.00-3.80 (m, 2H), 3.45 (s, 3H), 2.90-2.80 (m, 2H), 2.75-2.50 (m, 2H), 2.48-2.05 (m, 6H). |
| 19 | 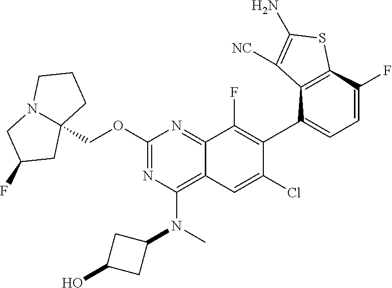 | LCMS m/z [M + 1]: 663.0 1H NMR (400 MHz, CD3OD) δ 8.24 (s, 1H), 7.23- 7.26 (m, 1H), 7.02-7.06 (t, 1H), 5.40 (m, 0.5H), 5.26 (m, 0.5H), 4.32-4.35 (m, 2H), 4.24-4.26 (m, 1H), 4.09-4.12 (m, 1H), 3.45 (s, 3H), 3.22-3.27 (m, 3H), 3.03-3.05 (m, 1H), 2.88-2.89 (m, 2H), 2.15-2.36 (m, 6H), 1.92-2.04 (m, 3H). |
| 20 | 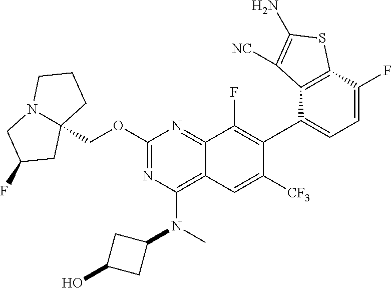 | LCMS m/z [M + 1]: 663.1 1H NMR (400 MHz, CD3OD) δ 8.24 (s, 1H), 7.23- 7.26 (m, 1H), 7.02-7.06 (t, 1H), 5.40 (m, 0.5H), 5.26 (m, 0.5H), 4.32-4.37 (m, 2H), 4.24-4.26 (m, 1H), 4.09-4.12 (m, 1H), 3.45 (s, 3H), 3.22-3.27 (m, 3H), 3.03-3.05 (m, 1H), 2.85-2.91 (m, 2H), 2.15-2.36 (m, 6H), 1.92-2.04 (m, 3H). |
| 20a | 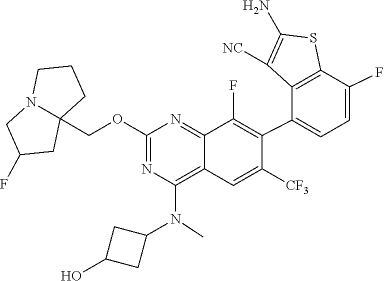 [SMILES: CN(c1nc(OCC23CCCN2CC(F)C3)nc2c(F)c(-c3ccc(F)c4sc(N)c(C#N)c34)c(C(F)(F)F)cc12)C1CC(O)C1] | |
| 22 | 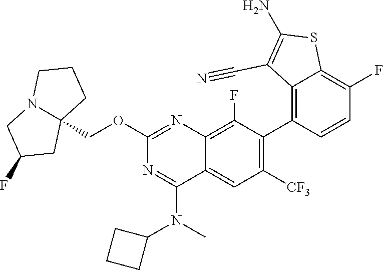 [SMILES: CN(c1nc(OC[C@@]23CCCN2C[C@H](F)C3)nc2c(F)c(-c3ccc(F)c4sc(N)c(C#N)c34)c(C(F)(F)F)cc12)C1CCC1] | LCMS m/z [M + 1]: 647.1 1H NMR (400 MHz, CD3OD) δ 8.27 (s, 1H), 7.23- 7.26 (m, 1H), 7.04 (t, 1H), 5.63 (s, 0.5H), 5.50 (s, 0.5H), 4.85 (d, 1H), 4.73 (t, 1H), 4.60-4.68 (m, 1H), 3.99-4.10 (m, 1H), 3.85-3.94 (m, 2H), 3.42-3.51 (m, 3H), 3.14 (s, 1H), 2.30-2.64 (m, 8H), 2.15 (s, 2H), 1.80-1.90 (m, 2H). |
| 22a | 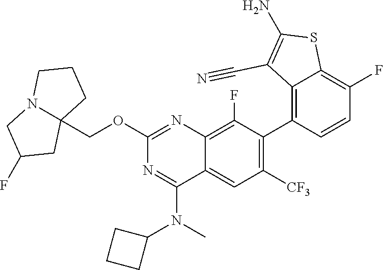 [SMILES: CN(c1nc(OCC23CCCN2CC(F)C3)nc2c(F)c(-c3ccc(F)c4sc(N)c(C#N)c34)c(C(F)(F)F)cc12)C1CCC1] | |
| 23 | 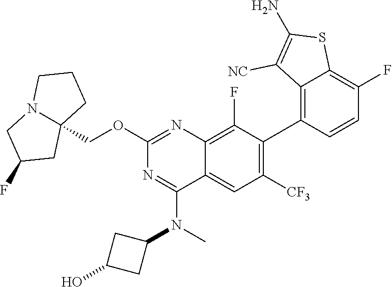 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@H]1C[C@H](O)C1] | LCMS m/z [M + 1]: 663.0 1H NMR (400 MHz, CD3OD) δ 8.2 (s, 1H), 7.24- 7.20 (m, 1H), 7.04-6.99 (m, 1H), 5.50 (s, 0.6H), 5.37 (s, 0.6H), 5.14-5.06 (m, 1H), 4.56-4.42 (m, 4H), 3.72-3.50 (m, 4H), 3.43 (s, 3H), 2.73-2.66 (m, 2H), 2.57-2.36 (m, 5H), 2.29-1.93 (m, 6H). |
| 23a | 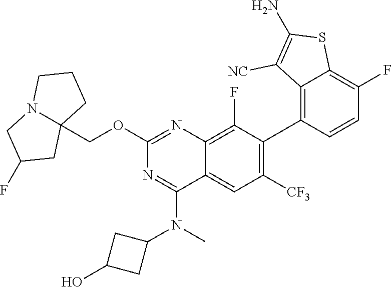 [SMILES: CN(c1nc(OCC23CCCN2CC(F)C3)nc2c(F)c(-c3ccc(F)c4sc(N)c(C#N)c34)c(C(F)(F)F)cc12)C1CC(O)C1] | |
| 24 | 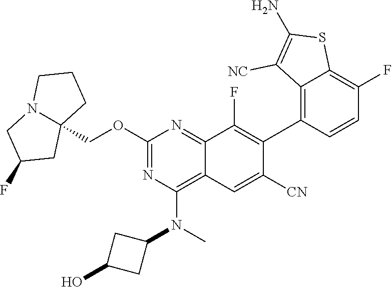 | LCMS m/z [M + 1]: 629.1 1H NMR (400 MHz, CD3OD) δ 8.51 (s, 1H), 7.98 (s, 1H), 7.23-7.19 (m, 1H), 7.06-7.02 (m, 1H), 5.49 (s, 0.52H), 5.35 (s, 0.59H), 4.52-4.24 (m, 3H), 4.11- 4.04 (m, 1H), 3.67-3.48 (m, 3H), 3.40 (s, 3H), 3.32 (d, J = 4.8 Hz, 1H), 2.87 (d, J = 8.8 Hz, 2H), 2.54-2.01 (m, 9H). |
| 24a | 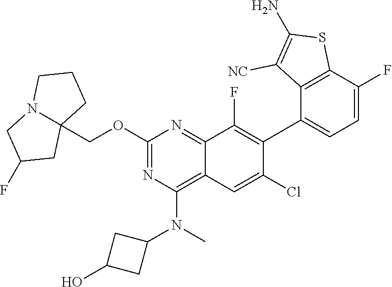 [SMILES: CN(c1nc(OCC23CCCN2CC(F)C3)nc2c(F)c(-c3ccc(F)c4sc(N)c(C#N)c34)c(Cl)cc12)C1CC(O)C1] | |
| 25 | 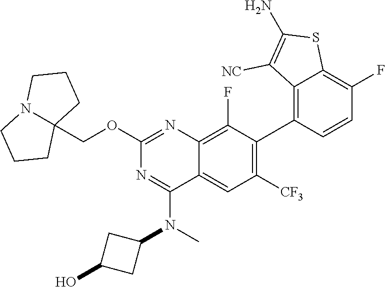 [SMILES: CN(c1nc(OCC23CCCN2CCC3)nc2c(F)c(-c3ccc(F)c4sc(N)c(C#N)c34)c(C(F)(F)F)cc12)[C@H]1C[C@@H](O)C1] | LCMS m/z [M + 1]: 645.1 1H NMR (400 MHz, CD3OD) δ 8.50 (d, 1H), 8.26 (s, 1H), 7.21-7.24 (m, 1H), 7.00-7.05 (t, 1H), 4.60- 4.69 (m, 2H),4.36 (m, 1H), 4.07-4.10 (m, 1H), 3.67- 3.71 (m, 2H), 3.45 (s, 3H), 3.25-3.28 (m, 2H), 2.84- 2.86 (m, 2H), 2.08-2.34 (m, 10H). |
| 25a | 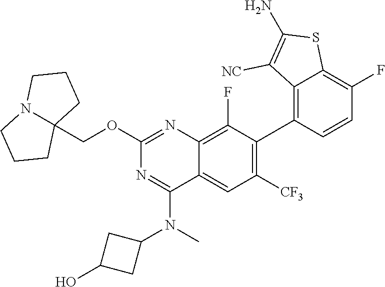 [SMILES: CN(c1nc(OCC23CCCN2CCC3)nc2c(F)c(-c3ccc(F)c4sc(N)c(C#N)c34)c(C(F)(F)F)cc12)C1CC(O)C1] | |
| 26 | 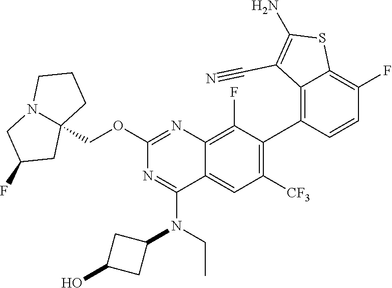 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@H]1C[C@@H](O)C1] | LCMS m/z [M + 1]: 677.2 1H NMR (400 MHz, CD3OD) δ 8.24 (s, 1H), 7.22- 7.20 (m, 1H), 7.02 (t, 1H), 5.54 (d, 1H), 4.74-4.58 (m, 3H), 4.41-4.29 (m, 1H), 4.07-3.85 (m, 6H), 2.86 (s, 2H), 2.41-2.27 (m, 4H), 1.70-1.62 (m, 2H), 1.38 (t, 3H), 1.02 (t, 2H). |
| 26a | 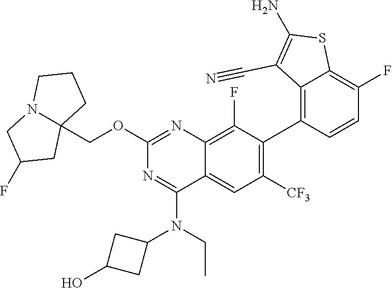 [SMILES: CCN(c1nc(OCC23CCCN2CC(F)C3)nc2c(F)c(-c3ccc(F)c4sc(N)c(C#N)c34)c(C(F)(F)F)cc12)C1CC(O)C1] | |
| 27 | 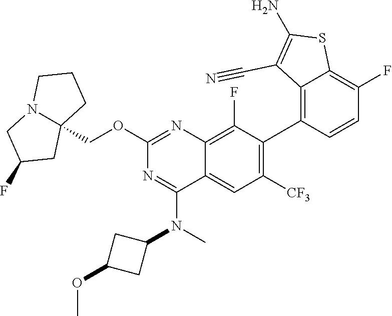 [SMILES: CO[C@H]1C[C@@H](N(C)c2nc(OC[C@@]34CCCN3C[C@H](F)C4)nc3c(F)c(-c4ccc(F)c5sc(N)c(C#N)c45)c(C(F)(F)F)cc23)C1] | LCMS m/z [M + 1]: 677.2 1H NMR (400 MHz, CD3OD) δ 8.23 (s, 1H), 7.23- 7.19 (m, 1H), 7.01 (t, 1H), 5.40 (d, 1H), 4.49-4.36 (m, 3H), 3.81-3.74 (m, 1H), 3.51 (s, 3H), 3.42 (s, 3H), 3.24-3.16 (m, 2H), 2.90-2.80 (m, 2H), 2.51-1.98 (m, 10H). |
| 27a | 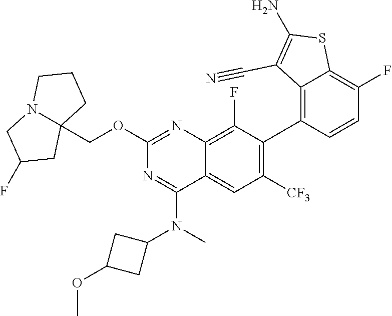 [SMILES: COC1CC(N(C)c2nc(OCC34CCCN3CC(F)C4)nc3c(F)c(-c4ccc(F)c5sc(N)c(C#N)c45)c(C(F)(F)F)cc23)C1] | |
| 28 | 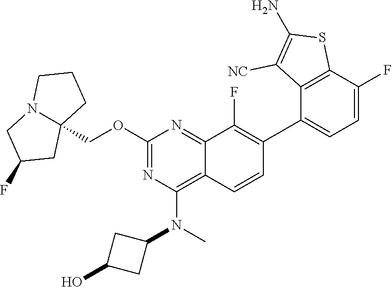 [SMILES: CN(c1nc(OC[C@@]23CCCN2C[C@H](F)C3)nc2c(F)c(-c3ccc(F)c4sc(N)c(C#N)c34)ccc12)[C@H]1C[C@@H](O)C1] | LCMS m/z [M + 1]: 595 1H NMR (400 MHz, CD3OD) δ 8.41 (s, 1H), 7.95 (d, J = 8.4 Hz, 1H), 7.41-7.21 (m, 2H), 7.04 (t, J = 7.2 Hz, 1H), 5.61-5.38 (m, 1H), 4.76-4.65 (m, 1H), 4.64-4.48 (m, 1H), 4.41-4.29 (m, 1H), 4.13-4.01 (m, 1H), 4.01-3.72 (m, 3H), 3.47-3.36 (m, 3H), 2.93- 2.75 (m, 2H), 2.75-1.88 (m, 8H). |
| 28a | 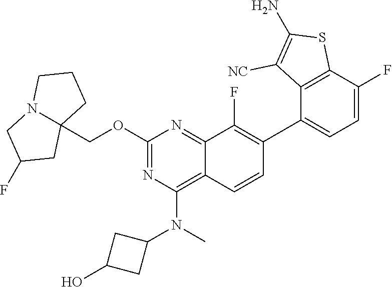 [SMILES: CN(c1nc(OCC23CCCN2CC(F)C3)nc2c(F)c(-c3ccc(F)c4sc(N)c(C#N)c34)ccc12)C1CC(O)C1] | |
| 29 | 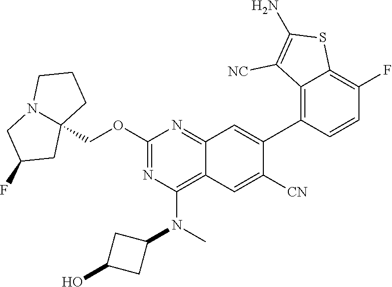 [SMILES: CN(c1nc(OC[C@@]23CCCN2C[C@H](F)C3)nc2cc(-c3ccc(F)c4sc(N)c(C#N)c34)c(C#N)cc12)[C@H]1C[C@@H](O)C1] | LCMS m/z [M + 1]: 602.3 1H NMR (400 MHz, CD3OD) δ 8.51 (s, 1H), 7.55 (s, 1H), 7.30-7.27 (m, 1H), 7.07-7.03 (m, 1H), 5.56- 5.43 (d, 1H), 4.57-4.45 (m, 2H), 4.39-4.31(m, 1H), 4.13-4.06 (m, 1H), 3.81-3.67 (m, 3H), 3.46 (s, 3H), 2.90-2.83 (m, 2H), 2.62-2.31 (m, 4H), 2.23-2.08 (m, 5H). |
| 29a | 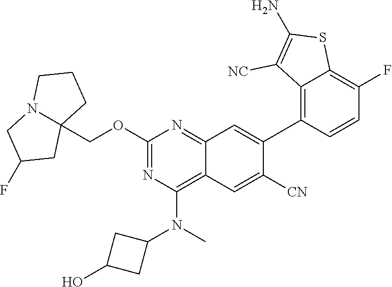 [SMILES: CN(c1nc(OCC23CCCN2CC(F)C3)nc2cc(-c3ccc(F)c4sc(N)c(C#N)c34)c(C#N)cc12)C1CC(O)C1] | |
| 30 | 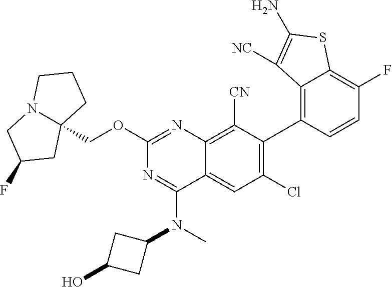 [SMILES: CN(c1nc(OC[C@@]23CCCN2C[C@H](F)C3)nc2c(C#N)c(-c3ccc(F)c4sc(N)c(C#N)c34)c(Cl)cc12)[C@H]1C[C@@H](O)C1] | LCMS m/z [M + 1]: 636 1H NMR (400 MHz, CD3OD) δ 8.39 (s, 1H), 7.25- 7.28 (m, 1H), 7.08 (t, 1H), 5.61 (s, 0.5H), 5.48 (s, 0.5H), 4.60-4.77 (m, 2H), 4.29-4.33 (m, 1H), 3.87- 4.09 (m, 4H), 3.42-3.47 (m, 4H), 2.82-2.87 (m, 2H), 2.58-2.63 (m, 2H), 2.43-2.46 (m, 1H), 2.33-2.36 (m, 2H), 2.19-2.20 (m, 3H). |
| 30a | 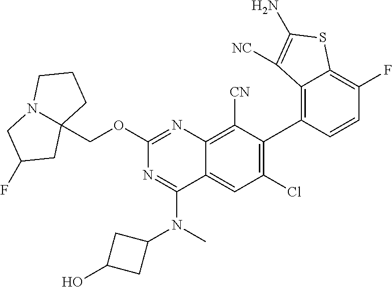 [SMILES: CN(c1nc(OCC23CCCN2CC(F)C3)nc2c(C#N)c(-c3ccc(F)c4sc(N)c(C#N)c34)c(Cl)cc12)C1CC(O)C1] | |
| 31 | 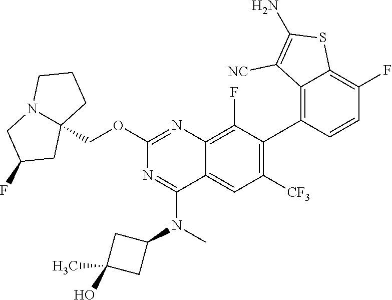 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@H]1C[C@@](C)(O)C1] | LCMS m/z [M + 1]: 677 1H NMR (400 MHz, CD3OD) δ 8.23 (s, 1H), 7.24- 7.21 (m, 1H), 7.04-7.00 (m, 1H), 5.48-5.37 (d, 1H), 4.54-4.38 (m, 3H), 3.67-3.48 (m, 3H), 3.43 (s, 3H), 3.26-3.16 (m, 1H), 2.58-2.00 (m, 10H), 1.37 (s, 3H). |
| 31a | 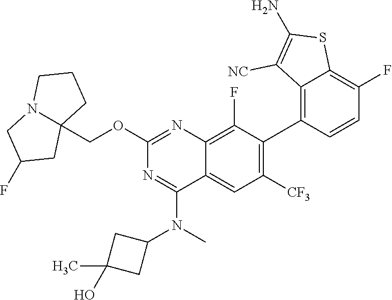 [SMILES: CN(c1nc(OCC23CCCN2CC(F)C3)nc2c(F)c(-c3ccc(F)c4sc(N)c(C#N)c34)c(C(F)(F)F)cc12)C1CC(C)(O)C1] | |
| 32 | 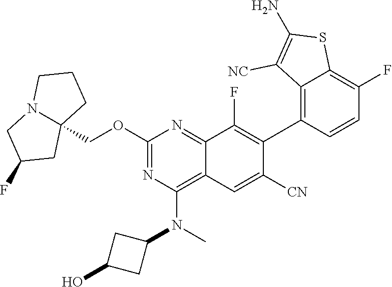 [SMILES: CN(c1nc(OC[C@@]23CCCN2C[C@H](F)C3)nc2c(F)c(-c3ccc(F)c4sc(N)c(C#N)c34)c(C#N)cc12)[C@H]1C[C@@H](O)C1] | LCMS m/z [M + 1]: 620.2 1H NMR (400 MHz, CD3OD) δ 8.36 (s, 1H), 7.34- 7.32 (s, 1H), 7.10-7.06 (s, 1H), 5.51 (s, 0.49H), 5.40 (s, 0.51H), 4.58-4.34 (m, 4H), 4.10-4.08 (m, 1H), 3.76-3.57 (m, 5H), 3.46 (s, 3H), 2.87 (s, 2H), 2.55- 2.40 (m, 3H), 2.32-2.21 (m, 6H). |
| 32a | 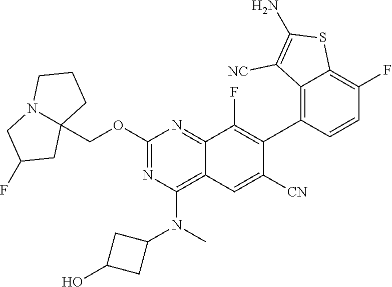 [SMILES: CN(c1nc(OCC23CCCN2CC(F)C3)nc2c(F)c(-c3ccc(F)c4sc(N)c(C#N)c34)c(C#N)cc12)C1CC(O)C1] | |
| 33 | 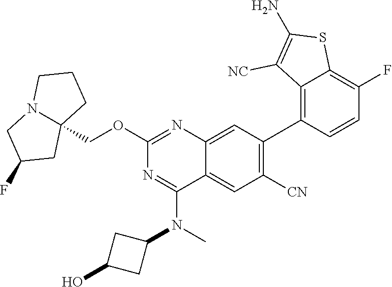 | LCMS m/z [M + 1]: 645.1 1H NMR (400 MHz, CD3OD) δ 8.37 (s, 1H), 7.45 (s, 1H), 7.19-7.18 (m, 1H), 6.98 (m, 1H), 5.42 (s, 0.5H), 5.29 (s, 0.5H), 4.39-4.27 (m, 3H), 4.10-4.03 (m, 1H), 3.48-3.33 (m, 6H), 3.12-3.08 (m, 1H), 2.87- 2.81 (m, 2H), 2.41-2.18 (m, 5H), 2.08-1.93 (m, 3H). |
| 33a | 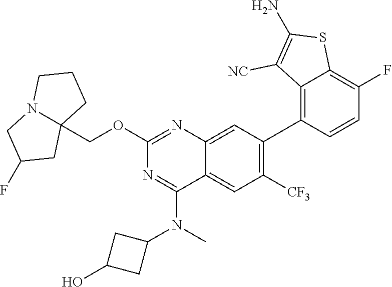 [SMILES: CN(c1nc(OCC23CCCN2CC(F)C3)nc2cc(-c3ccc(F)c4sc(N)c(C#N)c34)c(C(F)(F)F)cc12)C1CC(O)C1] | |
| 35 | 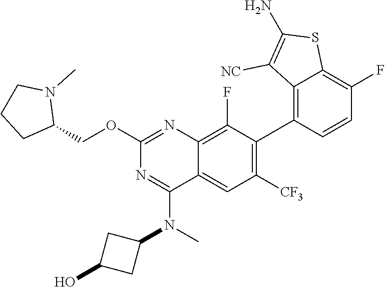 [SMILES: CN1CCC[C@H]1COc1nc(N(C)[C@H]2C[C@@H](O)C2)c2cc(C(F)(F)F)c(-c3ccc(F)c4sc(N)c(C#N)c34)c(F)c2n1] | LCMS m/z [M + 1]: 619.3 1H NMR (400 MHz, CD3OD) δ 8.24 (s, 1H), 7.20- 7.24 (m, 1H), 6.99-7.04 (t, 1H), 4.69-4.73 (m, 1H), 4.56-4.60 (m, 1H), 4.33-4.37 (m, 1H), 4.04-4.09 (t, 1H), 3.48-3.50 (m, 2H), 3.44 (s, 3H), 2.81-2.93 (m, 6H), 2.22-2.32 (m, 3H), 1.96-2.08 (m, 3H). |
| 35a | 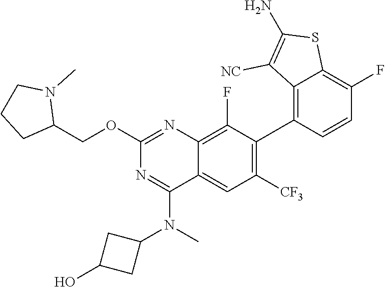 [SMILES: CN1CCCC1COc1nc(N(C)C2CC(O)C2)c2cc(C(F)(F)F)c(-c3ccc(F)c4sc(N)c(C#N)c34)c(F)c2n1] | |
| 36 | 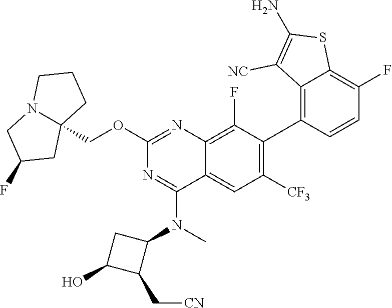 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@@H]1C[C@H](O)[C@@H]1CC#N] | |
| 36a | 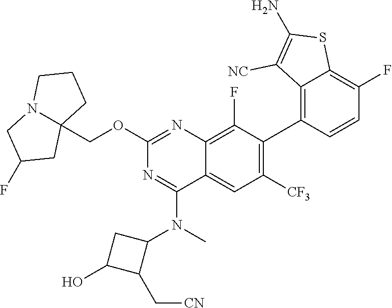 [SMILES: CN(c1nc(OCC23CCCN2CC(F)C3)nc2c(F)c(-c3ccc(F)c4sc(N)c(C#N)c34)c(C(F)(F)F)cc12)C1CC(O)C1CC#N] | |
| 37 | 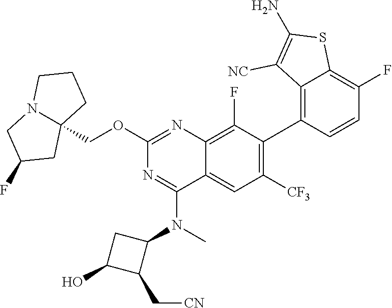 | LCMS m/z [M + 1]: 693.3 1H NMR (400 MHz, CD3OD) δ 8.36 (s, 1H), 7.23- 7.20 (m, 1H), 7.04-6.99 (m, 1H), 5.52 (s, 0.52H), 5.39 (s, 0.48H), 4.53-4.33 (m, 4H), 3.98-3.88 (m, 1H), 3.75-3.35 (m, 4H), 3.52 (d, J = 3.2 Hz, 3H), 2.85- 1.93 (m, 10H). |
| 37a | 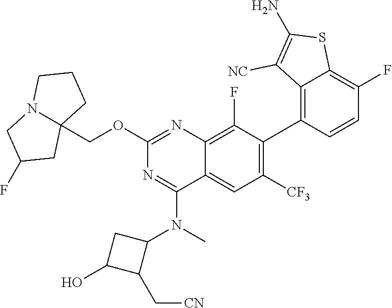 [SMILES: CN(c1nc(OCC23CCCN2CC(F)C3)nc2c(F)c(-c3ccc(F)c4sc(N)c(C#N)c34)c(C(F)(F)F)cc12)C1CC(O)C1CC#N] | |
| 38 | 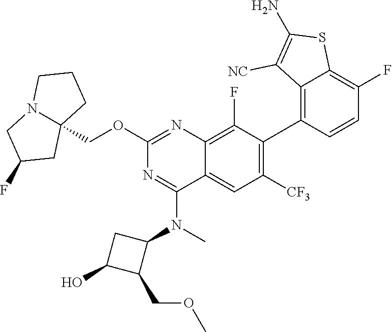 [SMILES: COC[C@@H]1[C@H](N(C)c2nc(OC[C@@]34CCCN3C[C@H](F)C4)nc3c(F)c(-c4ccc(F)c5sc(N)c(C#N)c45)c(C(F)(F)F)cc23)C[C@@H]1O] | |
| 38a | 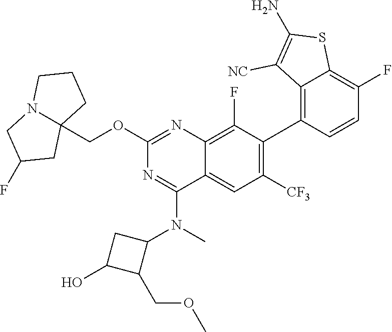 [SMILES: COCC1C(O)CC1N(C)c1nc(OCC23CCCN2CC(F)C3)nc2c(F)c(-c3ccc(F)c4sc(N)c(C#N)c34)c(C(F)(F)F)cc12] | |
| 39 | 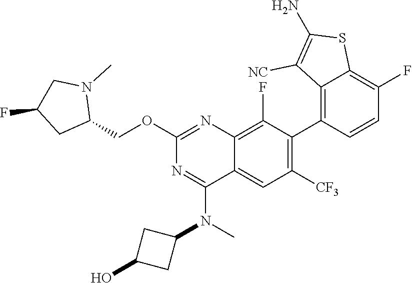 | LCMS m/z [M + 1]: 638.2 1H NMR (400 MHz, CD3OD) δ 8.24 (s, 1H), 7.22 (s, 1H), 7.05-6.99 (m, 1H), 5.38 (s, 0.58H), 5.25 (s, 0.57H), 4.73-4.68 (m, 2H), 4.36 (s, 1H), 4.08 (d, J = 4 Hz, 1H), 3.87-3.64 (m, 3H), 3.44 (d, J = 3.6 Hz, 3H), 3.14-3.05 (m, 2H), 2.84-2.82 (m, 6H), 2.51-2.42 (m, 1H), 2.25 (s, 3H). |
| 39a | 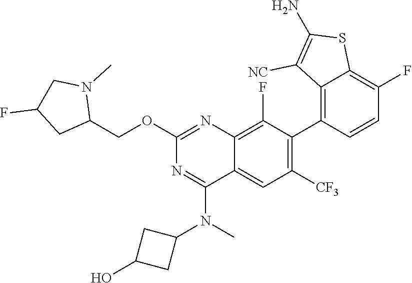 [SMILES: CN1CC(F)CC1COc1nc(N(C)C2CC(O)C2)c2cc(C(F)(F)F)c(-c3ccc(F)c4sc(N)c(C#N)c34)c(F)c2n1] | |
| 40 | 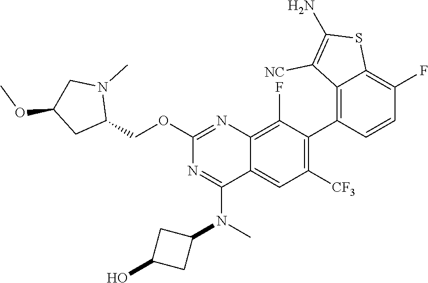 | LCMS m/z [M + 1]: 649.2 1H NMR (400 MHz, CD3OD) δ 8.23 (d, 1H), 7.21 (t, 1H), 7.01 (t, 1H), 4.79-4.75 (m, 2H), 4.61-4.57 (m, 1H), 4.37-4.31 (m, 1H), 4.11-4.03 (m, 2H), 3.76- 3.61 (m, 2H), 3.45-3.43 (m, 3H), 3.36-3.33 (m, 3H), 3.12 (d, 1H), 2.93 (d, 2H), 2.87-2.81 (m, 2H), 2.41- 2.36 (m, 1H), 2.27-2.16 (m, 2H), 2.13-2.06 (m, 1H). |
| 40a | 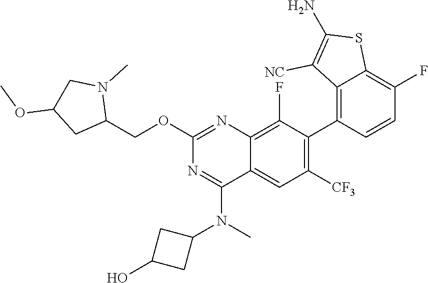 [SMILES: COC1CC(COc2nc(N(C)C3CC(O)C3)c3cc(C(F)(F)F)c(-c4ccc(F)c5sc(N)c(C#N)c45)c(F)c3n2)N(C)C1] | |
| 41 | 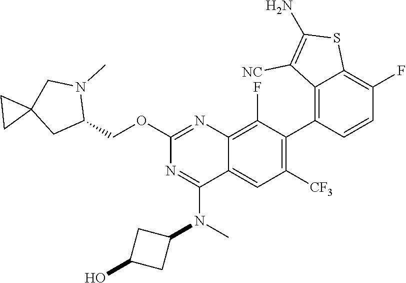 | LCMS m/z [M + 1]: 645.2 1H NMR (400 MHz, CD3OD) δ 8.25 (d, 1H), 7.23- 7.20 (m, 1H), 7.01 (t, 1H), 4.80-4.76 (m, 1H), 4.69- 4.64 (m, 1H), 4.39-4.31 (m, 1H), 4.11-4.03 (m, 1H), 3.85 (s, 1H), 3.44 (d, 3H), 3.26-3.17 (m, 2H), 2.96 (d, 3H), 2.87-2.81 (m, 2H), 2.40-2.22 (m, 3H), 1.94- 1.89 (m, 1H), 0.78-0.62 (m, 4H). |
| 41a | 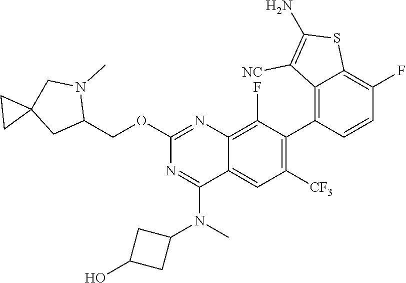 [SMILES: CN1CC2(CC2)CC1COc1nc(N(C)C2CC(O)C2)c2cc(C(F)(F)F)c(-c3ccc(F)c4sc(N)c(C#N)c34)c(F)c2n1] | |
| 42 | 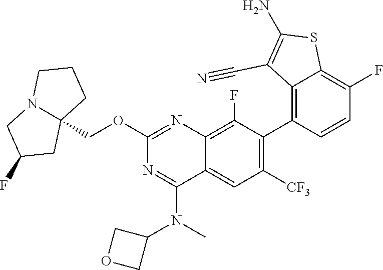 [SMILES: CN(c1nc(OC[C@@]23CCCN2C[C@H](F)C3)nc2c(F)c(-c3ccc(F)c4sc(N)c(C#N)c34)c(C(F)(F)F)cc12)C1COC1] | |
| 42a | 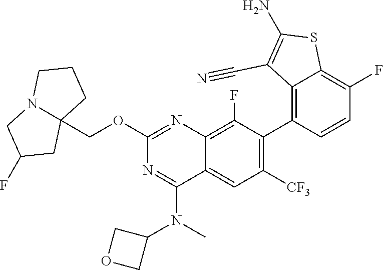 [SMILES: CN(c1nc(OCC23CCCN2CC(F)C3)nc2c(F)c(-c3ccc(F)c4sc(N)c(C#N)c34)c(C(F)(F)F)cc12)C1COC1] | |
| 43 | 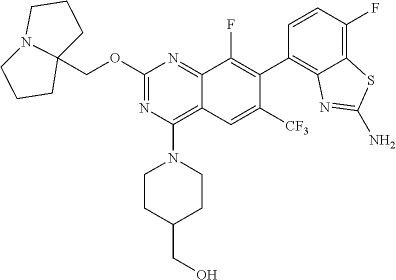 [SMILES: Nc1nc2c(-c3c(C(F)(F)F)cc4c(N5CCC(CO)CC5)nc(OCC56CCCN5CCC6)nc4c3F)ccc(F)c2s1] | LCMS m/z [M + 1]: 635.2 1H NMR (400 MHz, CD3OD): δ 8.18 (s, 1H), 7.25- 7.18 (m, 1H), 6.99 (t, J = 8.8 Hz, 1H), 4.65 (s, 4H), 3.70-3.60 (m, 2H), 3.50-3.45 (m, 2H), 3.45-3.35 (m, 2H), 3.30-3.25 (m, 2H), 2.30-1.95 (m, 11H), 1.60- 1.50 (m, 2H). |
| 44 | 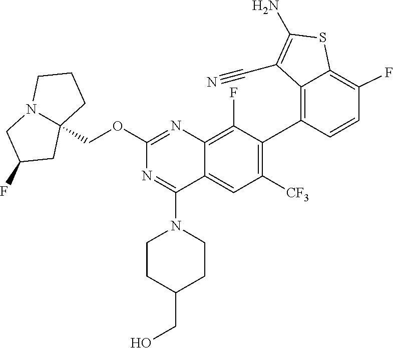 [SMILES: N#Cc1c(N)sc2c(F)ccc(-c3c(C(F)(F)F)cc4c(N5CCC(CO)CC5)nc(OC[C@@]56CCCN5C[C@H](F)C6)nc4c3F)c12] | LCMS m/z [M + 1]: 677.1 1H NMR (400 MHz, CD3OD) δ 8.16 (s, 1H), 7.21- 7.24 (m, 1H), 7.02 (t, 1H), 5.61 (s, 0.5H), 5.48 (s, 0.5H), 4.57-4.74 (m, 4H), 3.81-4.03 (m, 3H), 3.38- 3.51 (m, 5H), 3.52-3.74 (m, 2H), 2.33-2.41 (m, 3H), 2.14 (s, 1H), 1.96-1.99 (m, 3H), 1.47-1.53 (m, 2H). |
| 44a | 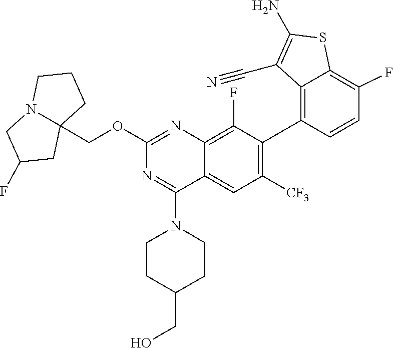 [SMILES: N#Cc1c(N)sc2c(F)ccc(-c3c(C(F)(F)F)cc4c(N5CCC(CO)CC5)nc(OCC56CCCN5CC(F)C6)nc4c3F)c12] | |
| 45 | 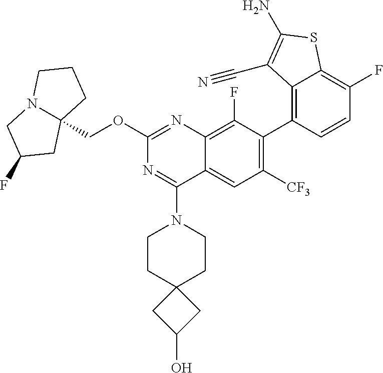 [SMILES: N#Cc1c(N)sc2c(F)ccc(-c3c(C(F)(F)F)cc4c(N5CCC6(CC5)CC(O)C6)nc(OC[C@@]56CCCN5C[C@H](F)C6)nc4c3F)c12] | LCMS m/z [M + 1]: 703 1H NMR (400 MHz, CD3OD) δ 8.16 (s, 1H), 7.22 (t, 1H), 7.02 (t, 1H), 5.61 (s, 0.5H), 5.48 (s, 0.5H), 4.58- 4.74 (m, 2H), 4.28-4.35 (m, 1H), 3.81-4.07 (m, 7H), 3.45 (s, 1H), 2.15-2.72 (m, 8H), 1.79-1.83 (m, 6H). |
| 45a | 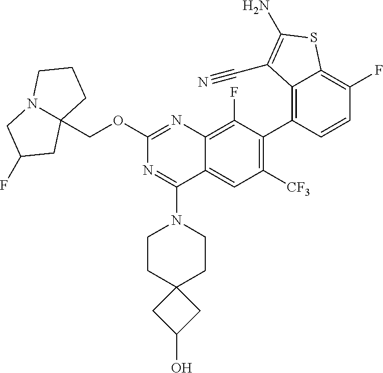 [SMILES: N#Cc1c(N)sc2c(F)ccc(-c3c(C(F)(F)F)cc4c(N5CCC6(CC5)CC(O)C6)nc(OCC56CCCN5CC(F)C6)nc4c3F)c12] | |
| 47 | 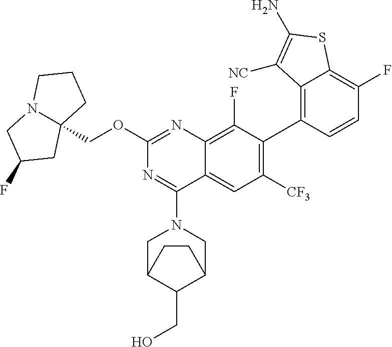 | LCMS m/z [M + 1]: 703.1 1HNMR (400 MHz, CD3OD) δ 8.21 (s, 1H), 7.21- 7.26 (m, 1H), 7.02 (t, 1H), 5.60-5.65 (m, 0.5H), 5.47- 5.52 (m, 0.5H), 4.55-4.80 (m, 4H), 3.95-4.10 (m, 1H), 3.80-3.95 (m, 2H), 3.55-3.68 (m, 2H), 3.35-3.50 (m, 3H), 2.65-2.78 (m, 1H), 2.56-2.63 (m, 1H), 2.42- 2.54 (m, 1H), 2.25-2.41 (m, 4H), 2.05-2.23 (m, 2H), 1.68-1.80 (m, 2H) 1.48-1.62 (m, 2H). |
| 47a | 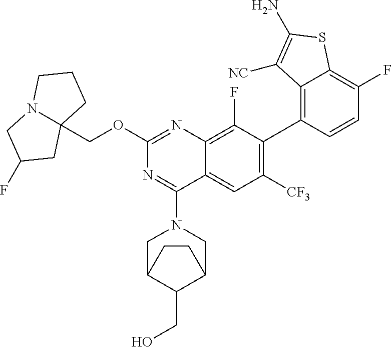 | |
| 48 | 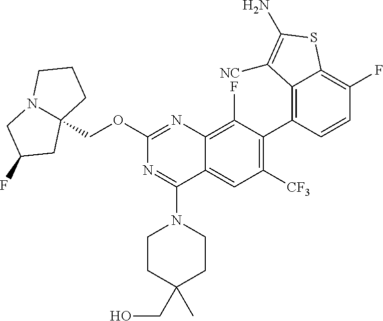 [SMILES: CC1(CO)CCN(c2nc(OC[C@@]34CCCN3C[C@H](F)C4)nc3c(F)c(-c4ccc(F)c5sc(N)c(C#N)c45)c(C(F)(F)F)cc23)CC1] | LCMS m/z [M + 1]: 691.1 1H NMR (400 MHz, CD3OD) δ 8.15 (s, 1H), 7.22- 7.19 (m, 1H), 7.00 (t, 1H), 5.43 (s, 0.5H), 5.29 (s, 0.5H), 4.44-4.30 (m, 2H), 4.19 (d, 2H), 3.79 (t, 2H), 3.41 (s, 3H), 3.12 (s, 1H), 2.46-1.95 (m, 8H), 1.81 (m, 2H), 1.53 (d, 2H), 1.11 (s, 3H). |
| 48a | 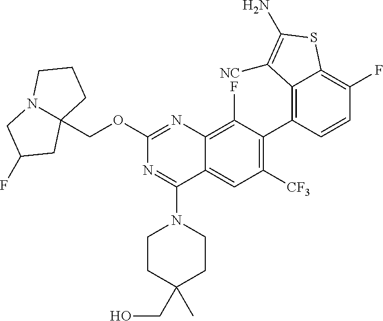 [SMILES: CC1(CO)CCN(c2nc(OCC34CCCN3CC(F)C4)nc3c(F)c(-c4ccc(F)c5sc(N)c(C#N)c45)c(C(F)(F)F)cc23)CC1] | |
| 49 | 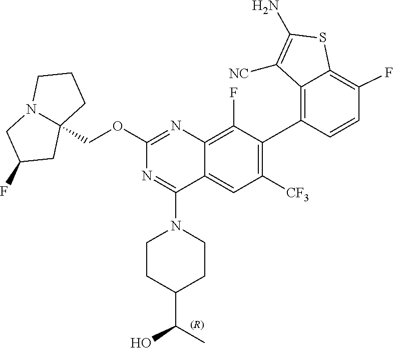 | LCMS m/z [M + 1]: 691.22 1H NMR (400 MHz, CD3OD) δ 8.14 (s, 1H), 7.29- 7.15 (m, 1H), 7.09-6.93 (m, 1H), 5.45-5.37 (m, 0.5H), 4.78-4.39 (m, 0.5H), 4.78-4.39 (m, 4H), 3.96- 3.50 (m, 4H), 3.41-3.30 (m, 5H), 2.72-1.98 (m, 7H), 1.92-1.48 (m, 4H), 1.34-1.08 (m, 3H). |
| 50 | 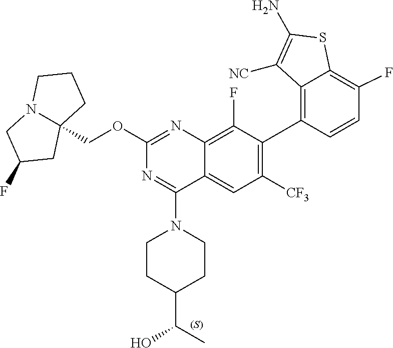 | LCMS m/z [M + 1]: 691.22 1H NMR (400 MHz, CD3OD) δ 8.13 (s, 1H), 7.24- 7.18 (m, 1H), 7.04-6.98 (m, 1H), 5.49 (s, 0.5H), 5.36 (s, 0.5H), 4.67-4.39 (m, 5H), 3.65-3.45 (m, 4H), 2.61-1.98 (m, 10H), 1.88-1.83 (d, J = 20 Hz, 1H), 1.74- 1.68 (m, 10H), 1.65-1.44 (m, 3H), 1.24-1.16 (m, 3H). |
| 50a | 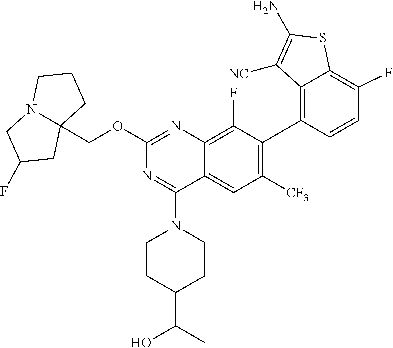 [SMILES: CC(O)C1CCN(c2nc(OCC34CCCN3CC(F)C4)nc3c(F)c(-c4ccc(F)c5sc(N)c(C#N)c45)c(C(F)(F)F)cc23)CC1] | |
| 51 | 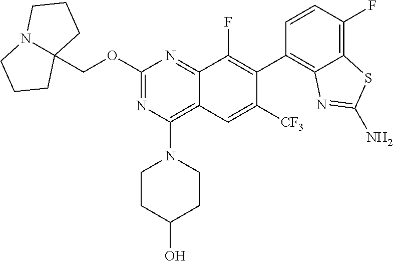 [SMILES: Nc1nc2c(-c3c(C(F)(F)F)cc4c(N5CCC(O)CC5)nc(OCC56CCCN5CCC6)nc4c3F)ccc(F)c2s1] | LCMS m/z [M + 1]: 621.2 1H NMR (400 MHz, CD3OD): δ 8.18 (s, 1H), 7.25- 7.18 (m, 1H), 6.99 (t, J = 8.4 Hz, 1H), 4.66 (s, 2H), 4.40-4.30 (m, 2H), 4.10-4.00 (m, 1H), 3.85-3.65 (m, 4H), 3.40-3.45 (m, 2H), 2.40-2.05 (m, 10H), 1.80- 1.70 (m, 2H). |
| 52 | 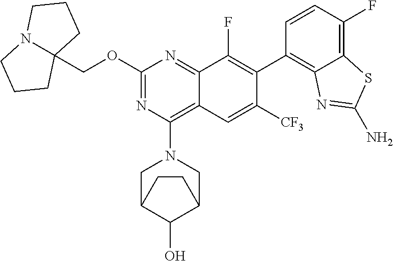 [SMILES: Nc1nc2c(-c3c(C(F)(F)F)cc4c(N5CC6CCC(C5)C6O)nc(OCC56CCCN5CCC6)nc4c3F)ccc(F)c2s1] | LCMS m/z [M + 1]: 647.2 1H NMR (400 MHz, CD3OD): δ 8.28 (s, 1H), 7.25- 7.18 (m, 1H), 6.99 (t, J = 8.4 Hz, 1H), 4.66 (s, 2H), 4.58-4.38 (m, 2H), 4.20-4.05 (m, 3H), 3.78-3.68 (m, 2H), 3.30-3.25 (m, 2H), 2.40-2.05 (m, 12H), 1.90- 1.80 (m, 2H). |
| 53 | 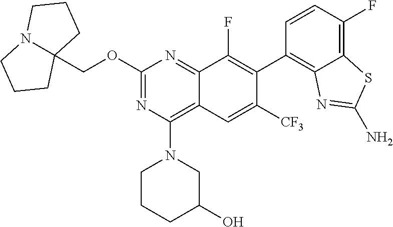 [SMILES: Nc1nc2c(-c3c(C(F)(F)F)cc4c(N5CCCC(O)C5)nc(OCC56CCCN5CCC6)nc4c3F)ccc(F)c2s1] | LCMS m/z [M + 1]: 621.2 1H NMR (400 MHz, CD3OD): δ 8.38 (s, 1H), 7.25- 7.18 (m, 1H), 6.99 (t, J = 8.4 Hz, 1H), 4.66 (s, 2H), 4.15-4.10 (m, 1H), 4.10-3.90 (m, 4H), 3.70-3.60 (m, 2H), 3.40-3.45 (m, 2H), 2.40-2.05 (m, 10H), 1.95- 1.80 (m, 2H). |
| 54 | 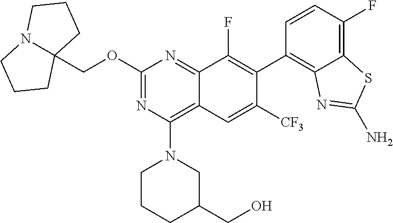 [SMILES: Nc1nc2c(-c3c(C(F)(F)F)cc4c(N5CCCC(CO)C5)nc(OCC56CCCN5CCC6)nc4c3F)ccc(F)c2s1] | LCMS m/z [M + 1]: 635.2 1H NMR (400 MHz, CD3OD): δ 8.23 (s, 1H), 7.20 (dd, J = 8.4, 5.6 Hz, 1H), 6.98 (t, J = 8.8 Hz, 1H), 4.75-4.65 (m, 3H), 4.55-4.45 (m, 1H), 3.78-3.68 (m, 2H), 3.65-3.55 (m, 1H), 3.50-3.40 (m, 3H), 3.30-3.25 (m, 2H), 2.40-1.95 (m, 11H), 1.90-1.75 (m, 1H), 1.58-1.48 (m, 1H). |
| 55 | 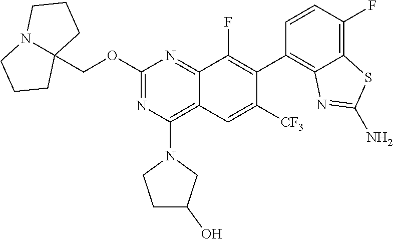 [SMILES: Nc1nc2c(-c3c(C(F)(F)F)cc4c(N5CCC(O)C5)nc(OCC56CCCN5CCC6)nc4c3F)ccc(F)c2s1] | LCMS m/z [M + 1]: 607.2 1H NMR (400 MHz, CD3OD): δ 8.51 (s, 1H), 7.28- 7.22 (m, 1H), 7.02 (t, J = 8.8 Hz, 1H), 4.71 (s, 2H), 4.65 (s, 1H), 4.40-4.15 (m, 3H), 4.10-4.00 (m, 1H), 3.78-3.68 (m, 2H), 3.30-3.25 (m, 2H), 2.40-1.95 (m, 10H). |
| 56 | 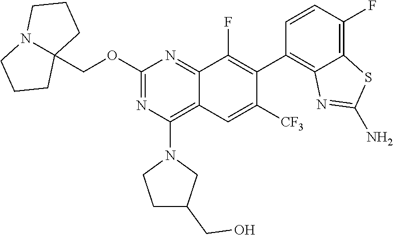 [SMILES: Nc1nc2c(-c3c(C(F)(F)F)cc4c(N5CCC(CO)C5)nc(OCC56CCCN5CCC6)nc4c3F)ccc(F)c2s1] | LCMS m/z [M + 1]: 621.2 1H NMR (400 MHz, CD3OD): δ 8.51 (s, 1H), 7.28- 7.20 (m, 1H), 7.00 (t, J = 8.8 Hz, 1H), 4.70 (s, 2H), 4.30-4.00 (m, 3H), 4.00-3.80 (m, 2H), 3.80-3.60 (m, 5H), 2.30-1.90 (m, 11H). |
| 57 | 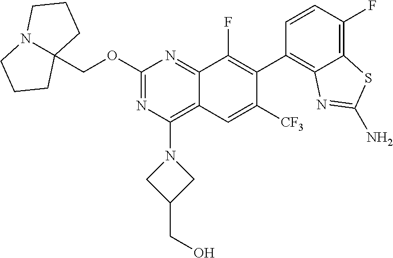 [SMILES: Nc1nc2c(-c3c(C(F)(F)F)cc4c(N5CC(CO)C5)nc(OCC56CCCN5CCC6)nc4c3F)ccc(F)c2s1] | LCMS m/z [M + 1]: 607.1 1H NMR (400 MHz, CD3OD): δ 8.14 (s, 1H), 7.25- 7.18 (m, 1H), 6.98 (t, J = 8.8 Hz, 1H), 4.64 (s, 2H), 4.65-4.40 (m, 1H), 4.40-4.20 (m, 1H), 4.10-4.00 (m, 1H), 3.85-3.80 (m, 2H), 3.75-3.65 (m, 2H), 3.30-3.25 (m, 2H), 3.15-3.05 (m, 1H), 2.35-1.95 (m, 9H). |
| 58 | 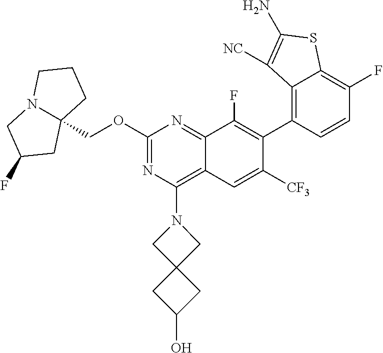 [SMILES: N#Cc1c(N)sc2c(F)ccc(-c3c(C(F)(F)F)cc4c(N5CC6(CC(O)C6)C5)nc(OC[C@@]56CCCN5C[C@H](F)C6)nc4c3F)c12] | LCMS m/z [M + 1]: 675.2 1H NMR (400 MHz, CD3OD): δ 8.09 (s, 1H), 7.25- 7.17 (m, 1H), 7.02 (t, J = 8.8 Hz, 1H), 5.54 (d, J = 51.6 Hz, 1H), 4.80-4.60 (m, 3H), 4.60-4.30 (m, 2H), 4.30-4.20 (m, 1H), 4.10-3.80 (m, 3H), 3.50-3.40 (m, 2H), 2.75-2.55 (m, 4H), 2.50-2.00 (m, 6H). |
| 58a | 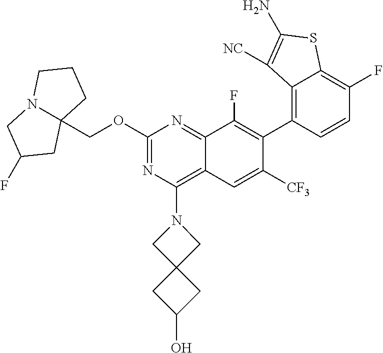 [SMILES: N#Cc1c(N)sc2c(F)ccc(-c3c(C(F)(F)F)cc4c(N5CC6(CC(O)C6)C5)nc(OCC56CCCN5CC(F)C6)nc4c3F)c12] | |
| 60 | 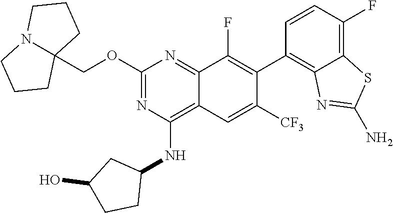 | LCMS m/z [M + 1]: 621.2 1H NMR (400 MHz, CD3OD): δ 8.58 (s, 1H), 7.21 (dd, J = 8.0, 5.6 Hz, 1H), 6.99 (t, J = 8.8 Hz, 1H), 4.85-4.75 (m, 1H), 4.68 (s, 2H), 4.55-4.45 (m, 1H), 3.78-3.68 (m, 2H), 3.30-3.25 (m, 2H), 2.60-1.70 (m, 14H). |
| 61 | 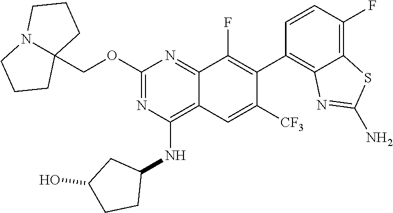 | LCMS m/z [M + 1]: 621.2 1H NMR (400 MHz, CD3OD): δ 8.62 (s, 1H), 7.20 (dd, J = 8.0, 5.6 Hz, 1H), 6.99 (t, J = 8.8 Hz, 1H), 4.85-4.75 (m, 1H), 4.68 (s, 2H), 4.55-4.45 (m, 1H), 3.78-3.68 (m, 2H), 3.30-3.25 (m, 2H), 2.60-1.70 (m, 14H). |
| 61a | 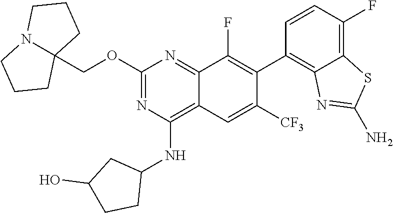 [SMILES: Nc1nc2c(-c3c(C(F)(F)F)cc4c(NC5CCC(O)C5)nc(OCC56CCCN5CCC6)nc4c3F)ccc(F)c2s1] | |
| 62 | 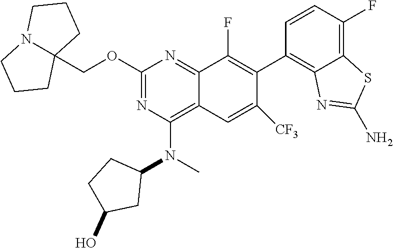 | LCMS m/z [M + 1]: 635.2 1H NMR (400 MHz, CD3OD): δ 8.34 (s, 1H), 7.20 (dd, J = 8.0, 5.6 Hz, 1H), 6.98 (t, J = 8.8 Hz, 1H), 5.20-5.10 (m, 1H), 4.65 (s, 2H), 4.40-4.30 (m, 1H), 3.78-3.68 (m, 2H), 3.50 (s, 3H), 3.30-3.25 (m, 2H), 2.50-2.00 (m, 11H), 1.90-1.80 (m, 3H). |
| 62a | 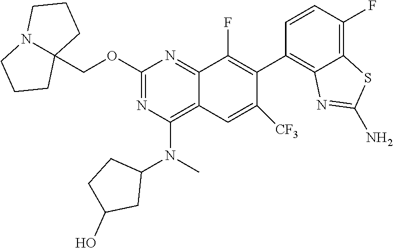 [SMILES: CN(c1nc(OCC23CCCN2CCC3)nc2c(F)c(-c3ccc(F)c4sc(N)nc34)c(C(F)(F)F)cc12)C1CCC(O)C1] | |
| 63 | 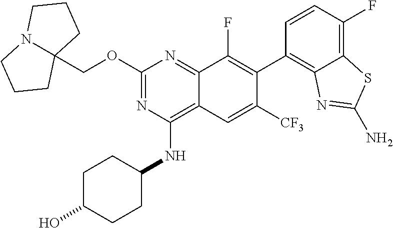 [SMILES: Nc1nc2c(-c3c(C(F)(F)F)cc4c(N[C@H]5CC[C@H](O)CC5)nc(OCC56CCCN5CCC6)nc4c3F)ccc(F)c2s1] | LCMS m/z [M + 1]: 635.2 1H NMR (400 MHz, CD3OD): δ 8.61 (s, 1H), 7.21 (dd, J = 8.0, 5.6 Hz, 1H), 6.99 (t, J = 8.8 Hz, 1H), 4.69 (s, 2H), 4.40-4.30 (m, 1H), 3.78-3.65 (m, 3H), 3.30-3.25 (m, 2H), 2.40-2.00 (m, 12H), 1.70-1.55 (m, 2H), 1.55-1.40 (m. 2H), 1.30-1.25 (m, 1H). |
| 64 | 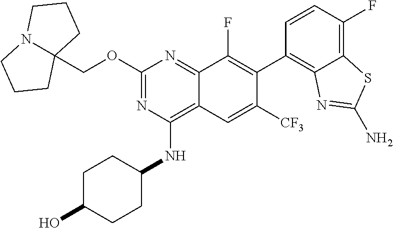 [SMILES: Nc1nc2c(-c3c(C(F)(F)F)cc4c(N[C@H]5CC[C@@H](O)CC5)nc(OCC56CCCN5CCC6)nc4c3F)ccc(F)c2s1] | LCMS m/z [M + 1]: 635.2 1H NMR (400 MHz, CD3OD): δ 8.65 (s, 1H), 7.21 (dd, J = 8.0, 5.6 Hz, 1H), 6.99 (t, J = 8.8 Hz, 1H), 4.68 (s, 2H), 4.40-4.28 (m, 1H), 4.02 (br s, 1H), 3.80-3.67 (m, 2H), 3.30-3.25 (m, 2H), 2.40-1.70 (m, 16H). |
| 64a | 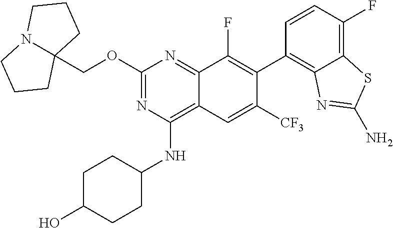 [SMILES: Nc1nc2c(-c3c(C(F)(F)F)cc4c(NC5CCC(O)CC5)nc(OCC56CCCN5CCC6)nc4c3F)ccc(F)c2s1] | |
| 65 | 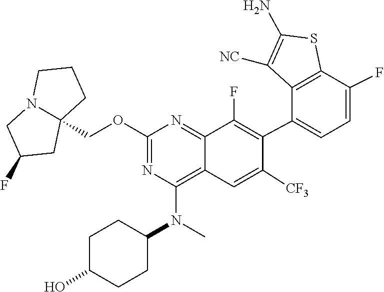 | LCMS m/z [M + 1]: 691 1H NMR (400 MHz, CD3OD) δ 8.33 (s, 1H), 7.24- 7.27 (m, 1H), 7.05 (t, 1H), 5.63 (s, 0.5H), 5.50 (s, 0.5H), 4.62-4.78 (m, 3H), 3.87-4.06 (m, 3H), 3.64- 3.69 (m, 1H), 3.42-3.46 (m, 3H), 1.90-2.73 (m, 13H), 1.45-1.54 (m, 2H). |
| 66 | 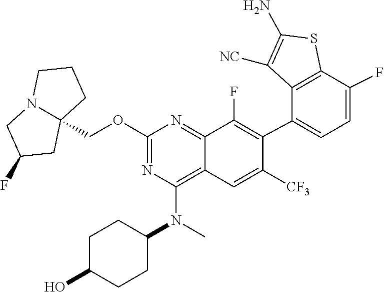 | LCMS m/z [M + 1]: 691.0 1H NMR (400 MHz, CD3OD) δ 8.30 (s, 1H), 7.21- 7.24 (m, 1H), 7.02 ( t, 1H), 5.61 (s, 0.5H), 5.47 (s, 0.5H), 4.51-4.71 (m, 3H), 3.87-4.03 (m, 4H), 3.42- 3.47 (m, 4H), 2.56-2.62 (m, 2H), 1.95-2.33 (m, 6H), 1.73-1.76 (m, 2H), 1.64-1.76 (m, 4H). |
| 66a | 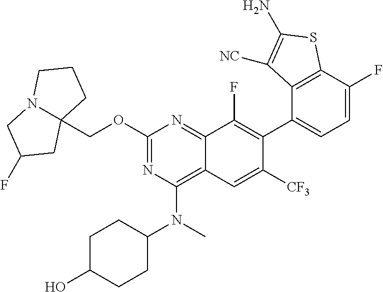 [SMILES: CN(c1nc(OCC23CCCN2CC(F)C3)nc2c(F)c(-c3ccc(F)c4sc(N)c(C#N)c34)c(C(F)(F)F)cc12)C1CCC(O)CC1] | |
| 67 | 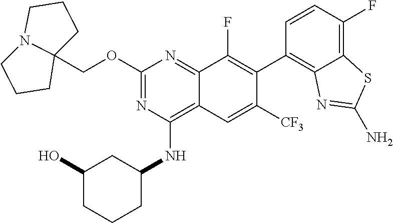 | LCMS m/z [M + 1]: 635.2 1H NMR (400 MHz, CD3OD): δ 8.58 (s, 1H), 7.23- 7.17 (m, 1H), 6.99 (t, J = 8.8 Hz, 1H), 4.66 (s, 2H), 4.47-4.37 (m, 1H), 3.80-3.60 (m, 4H), 2.40-1.90 (m, 12H), 1.60-1.48 (m, 3H), 1.40-1.30 (m, 1H). |
| 67a | 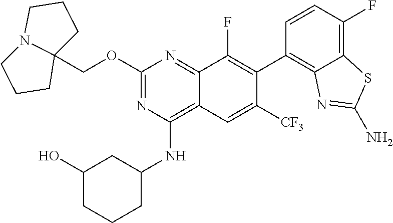 [SMILES: Nc1nc2c(-c3c(C(F)(F)F)cc4c(NC5CCCC(O)C5)nc(OCC56CCCN5CCC6)nc4c3F)ccc(F)c2s1] | |
| 68 | 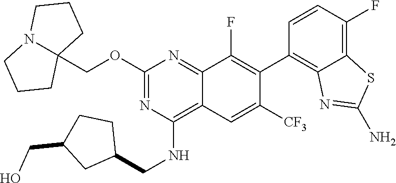 [SMILES: Nc1nc2c(-c3c(C(F)(F)F)cc4c(NC[C@@H]5CC[C@H](CO)C5)nc(OCC56CCCN5CCC6)nc4c3F)ccc(F)c2s1] | LCMS m/z [M + 1]: 649.2 1H NMR (400 MHz, CD3OD): δ 8.55 (s, 1H), 7.27- 7.20 (m, 1H), 6.99 (t, J = 8.8 Hz, 1H), 4.68 (s, 2H), 3.80-3.62 (m, 4H), 3.52-3.40 (m, 3H), 3.30-3.25 (m, 2H), 2.60-2.00 (m, 12H), 2.00-1.75 (m, 2H), 1.15- 1.05 (m, 1H). |
| 68a | 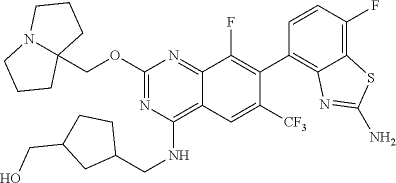 [SMILES: Nc1nc2c(-c3c(C(F)(F)F)cc4c(NCC5CCC(CO)C5)nc(OCC56CCCN5CCC6)nc4c3F)ccc(F)c2s1] | |
| 69 | 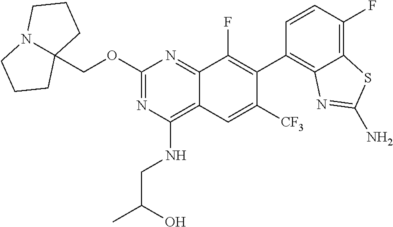 | LCMS m/z [M + 1]: 505.2 1H NMR (400 MHz, CD3OD): δ 8.54 (s, 1H), 7.20 (dd, J = 8.0, 5.6 Hz, 1H), 6.98 (t, J = 8.8 Hz, 1H), 4.67 (s, 2H), 4.25-4.15 (m, 1H), 3.80-3.65 (m, 4H), 3.30-3.25 (m, 2H), 2.40-2.05 (m, 8H), 1.28 (d, J = 6.0 Hz, 3H). |
| 70 | 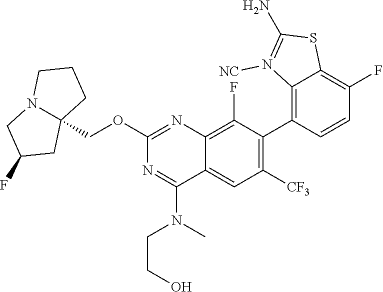 | LCMS m/z [M + 1]: 637.0 1H NMR (400 MHz, CD3OD) δ 8.65 (s, 1H), 7.21 (t, 1H), 7.02 (t, 1H), 5.61 (s, 0.5H), 5.48 (s, 0.5H), 4.74- 4.57 (m, 2H), 4.07-3.86 (m, 6H), 3.70-3.55 (m, 3H), 3.49-3.41 (m, 1H), 2.71-2.13 (m, 7H). |
| 70a | 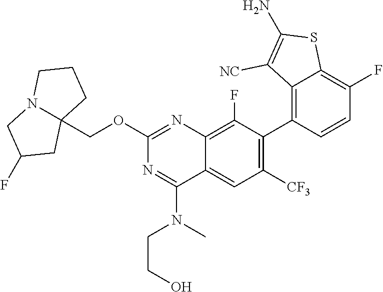 [SMILES: CN(CCO)c1nc(OCC23CCCN2CC(F)C3)nc2c(F)c(-c3ccc(F)c4sc(N)c(C#N)c34)c(C(F)(F)F)cc12] | |
| 71 | 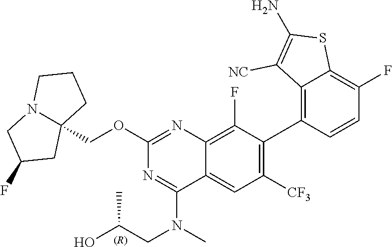 | LCMS m/z [M + 1]: 651.19 1H NMR (400 MHz, CD3OD) δ 8.67-8.57 (m, 1H), 7.29-7.16 (m, 1H), 7.11-6.95 (m, 1H), 5.51-5.28 (m, 1H), 4.55-4.24 (m, 3H), 3.97-3.77 (m, 2H), 3.68- 3.59 (m, 3H), 3.58-3.36 (m, 3H), 3.24-3.11 (m, 1H), 2.52-1.89 (m, 6H), 1.39-1.18 (m, 3H). |
| 72 | 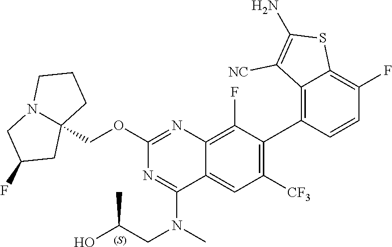 | LCMS m/z [M + 1]: 651.19 1H NMR (400 MHz, CD3OD) δ 8.67-8.60 (d,1H), 8.49 (s, 1H), 7.24-7.18 (m,1H), 7.06-6.98 (m,1H), 5.55-5.50 (m, 0.5H), 5.42-5.36 (m, 0.5H), 4.58-4.44 (m, 2H), 4.40-4.30 (m, 1H), 3.95-3.84 (m, 2H), 3.80- 3.57 (m, 6H), 6.30-3.25 (m, 1H), 2.60-2.00 (m, 7H), 1.32-1.24 (d, 3H). |
| 72a | 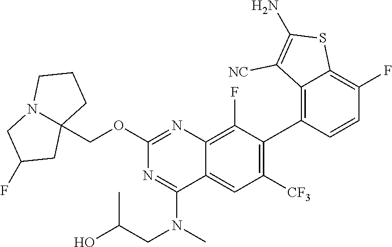 [SMILES: CC(O)CN(C)c1nc(OCC23CCCN2CC(F)C3)nc2c(F)c(-c3ccc(F)c4sc(N)c(C#N)c34)c(C(F)(F)F)cc12] | |
| 73 | 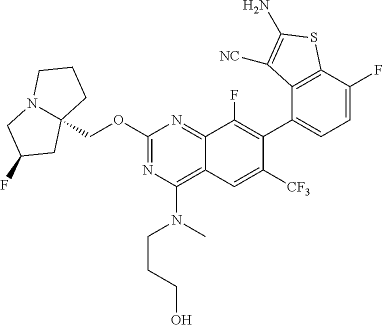 [SMILES: CN(CCCO)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 651.0 1H NMR (400 MHz, CD3OD) δ 8.43 (s, 1H), 7.20- 7.23 (m, 1H), 7.02 (t, 1H), 5.61 (s, 0.5H), 5.48 (s, 0.5H), 4.59-4.75 (m, 2H), 3.87-4.06 (m, 5H), 3.71 (t, 2H), 3.61 (s, 3H), 3.47 (m, 1H), 2.33-2.68 (m, 5H), 2.06-2.09 (m, 3H). |
| 73a | 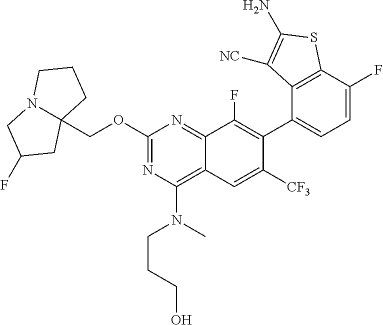 [SMILES: CN(CCCO)c1nc(OCC23CCCN2CC(F)C3)nc2c(F)c(-c3ccc(F)c4sc(N)c(C#N)c34)c(C(F)(F)F)cc12] | |
| 74 | 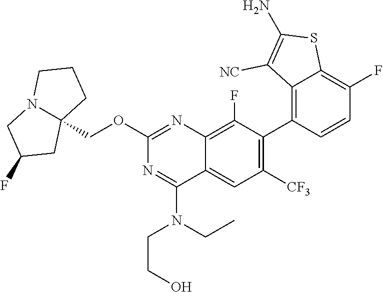 [SMILES: CCN(CCO)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 651.3 1H NMR (400 MHz, CD3OD) δ 8.54 (s, 1H), 7.20- 7.23 (m, 1H), 6.99-7.04 (m, 1H), 5.53 (m, 0.5H), 5.40 (m, 0.5H), 4.45-4.59 (m, 2H), 3.97-4.00 (m, 4H), 3.66 (m, 2H), 2.03-2.57 (m, 10H), 1.48-1.52 (m, 3H). |
| 74a | 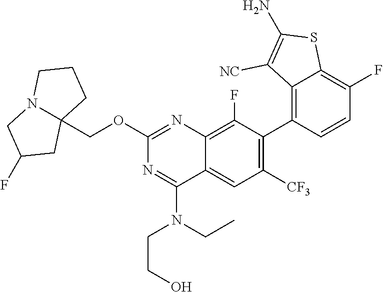 [SMILES: CCN(CCO)c1nc(OCC23CCCN2CC(F)C3)nc2c(F)c(-c3ccc(F)c4sc(N)c(C#N)c34)c(C(F)(F)F)cc12] | |
| 75 | 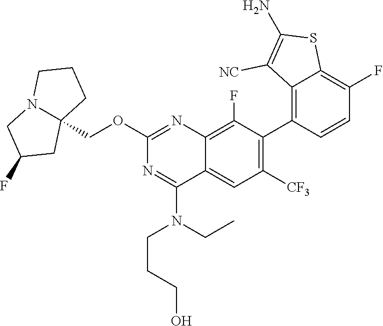 [SMILES: CCN(CCCO)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 665.3 1H NMR (400 MHz, CD3OD) δ 8.23 (s, 1H), 7.22- 7.19 (m, 1H), 7.00 (t, 1H), 5.38 (s, 0.5H), 5.25 (s, 0.5H), 4.35-4.24 (m, 2H), 3.96-3.863 (m, 4H), 3.71 (t, 2H), 3.36 (d, 1H), 3.25-3.20 (m, 1H), 3.05-3.01 (m, 1H), 2.41-1.88 (m, 9H), 1.50 (t, 3H). |
| 75a | 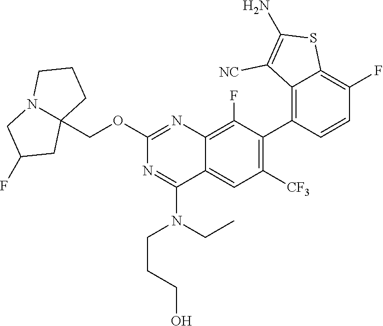 [SMILES: CCN(CCCO)c1nc(OCC23CCCN2CC(F)C3)nc2c(F)c(-c3ccc(F)c4sc(N)c(C#N)c34)c(C(F)(F)F)cc12] | |
| 76 | 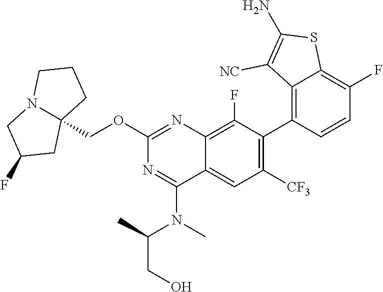 [SMILES: C[C@H](CO)N(C)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 651.0 1H NMR (400 MHz, CD3OD) δ 8.49 (s, 1H), 7.23- 7.19 (m, 1H), 7.04-6.99 (m, 1H), 5.49-5.45 (m, 0.5H), 5.35-5.34 (m, 0.5H), 4.99-4.94 (m, 1H), 4.50- 4.38 (m, 2H), 3.91-3.85 (m, 1H), 3.73-3.49 (m, 4H), 3.37-3.36 (m, 3H), 3.22-3.20 (m, 1H) 2.42-1.99 (m, 7H), 1.36-1.29 (m, 5H). |
| 77 | 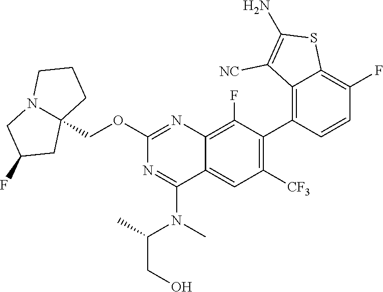 [SMILES: C[C@@H](CO)N(C)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 652.2 1H NMR (400 MHz, CD3OD) δ 8.49 (s, 1H), 7.24- 7.20 (m, 1H), 7.05-7.00 (m, 1H), 5.47-5.46 (m, 0.5H), 5.34-5.32 (m, 0.5H), 5.03-4.95 (m, 1H), 4.50- 4.33 (m, 2H), 3.91-3.86 (m, 1H), 3.73-3.67 (m, 1H), 3.64-3.44 (m, 3H), 3.38-3.37 (m, 3H), 3.20-3.17 (m, 1H), 2.36-2.10 (m, 4H), 2.10-1.97 (m, 4H) 1.35-1.32 (m, 3H). |
| 77a | 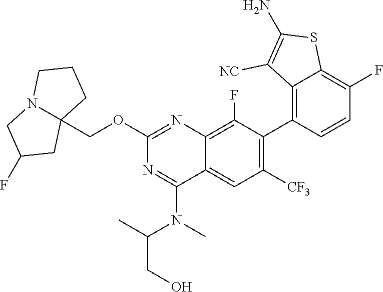 [SMILES: CC(CO)N(C)c1nc(OCC23CCCN2CC(F)C3)nc2c(F)c(-c3ccc(F)c4sc(N)c(C#N)c34)c(C(F)(F)F)cc12] | |
| 78 | 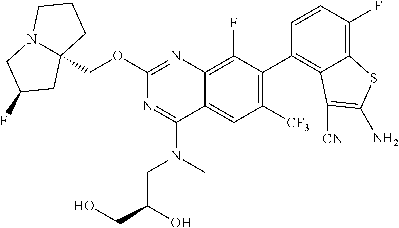 [SMILES: CN(C[C@@H](O)CO)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 667.1 1H NMR (400 MHz, CD3OD) δ 8.61 (s,1H), 7.23- 7.19 (m,1H), 7.04-6.98 (m,1H), 5.53-5.50 (m,0.5H), 5.40-5.37 (m, 0.5H), 4.58-4.45 (m, 2H), 4.22-4.10 (m, 2H), 3.92-3.84 (m,1H), 3.72-3.57 (m, 7H), 2.59- 2.32 (m, 3H), 2.23-1.99 (m, 4H), 1.47-1.39 (m, 1H). |
| 79 | 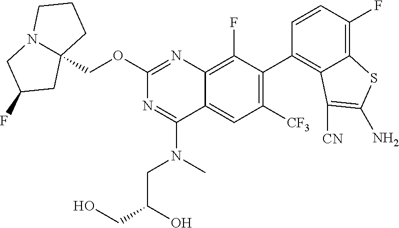 [SMILES: CN(C[C@H](O)CO)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 667 1H NMR (400 MHz, CD3OD) δ 8.62 (s, 1H), 7.23- 7.20 (m, 1H), 7.04-7.00 (m, 1H), 5.53-5.40 (m, 1H), 4.60-4.47 (m, 2H), 4.22-4.12 (m, 2H), 3.91-3.76 (m, 1H), 3.73-3.58 (m, 6H), 3.28-3.22 (m, 1H), 2.62-2.04 (m, 6H), 1.70-1.62 (m, 1H), 1.47-1.37 (m, 1H). |
| 79a | 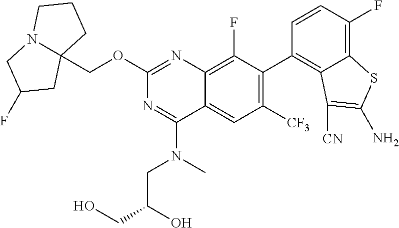 [SMILES: CN(C[C@H](O)CO)c1nc(OCC23CCCN2CC(F)C3)nc2c(F)c(-c3ccc(F)c4sc(N)c(C#N)c34)c(C(F)(F)F)cc12] | |
| 80 | 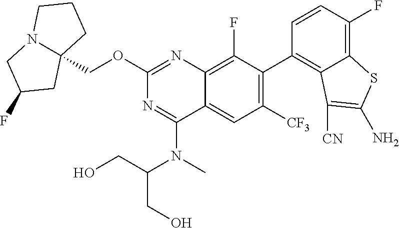 [SMILES: CN(c1nc(OC[C@@]23CCCN2C[C@H](F)C3)nc2c(F)c(-c3ccc(F)c4sc(N)c(C#N)c34)c(C(F)(F)F)cc12)C(CO)CO] | LCMS m/z [M + 1]: 667 1H NMR (400 MHz, CD3OD) δ 8.80 (s, 1H), 7.23- 7.20 (m, 1H), 7.04-7.00 (m, 1H), 5.55 (m, 0.5H), 5.42 (m, 0.5H), 4.98 (m, 1H), 4.74-4.44 (m, 2H), 3.98-3.79 (m, 4H), 3.70 (m, 2H), 3.43 (s, 3H), 3.42- 1.94 (m, 8H). |
| 80a | 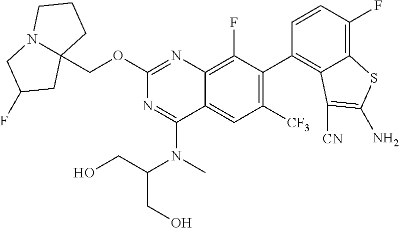 [SMILES: CN(c1nc(OCC23CCCN2CC(F)C3)nc2c(F)c(-c3ccc(F)c4sc(N)c(C#N)c34)c(C(F)(F)F)cc12)C(CO)CO] | |
| 81 | 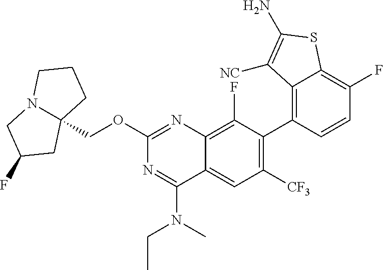 [SMILES: CCN(C)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 621.3 1H NMR (400 MHz, CD3OD) δ 8.32 (s, 1H), 7.22- 7.19 (m, 1H), 7.03-6.90 (m, 1H), 5.44 (s, 0.5H), 5.31 (s, 0.5H), 4.42-4.31 (m, 2H), 3.96-3.87 (m, 3H), 3.51 (s, 3H), 3.46-3.37 (m, 2H), 3.16-3.09 (m, 1H), 2.40- 2.16 (m, 3H), 2.14-2.02 (m, 2H), 1.98-1.90 (m, 1H), 1.44 (t, 3H). |
| 81a | 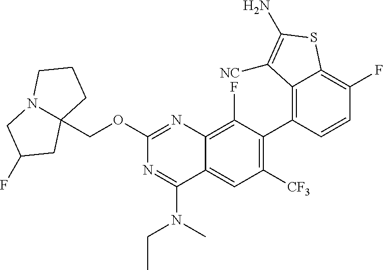 [SMILES: CCN(C)c1nc(OCC23CCCN2CC(F)C3)nc2c(F)c(-c3ccc(F)c4sc(N)c(C#N)c34)c(C(F)(F)F)cc12] | |
| 82 | 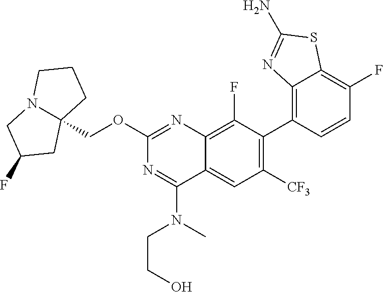 [SMILES: CN(CCO)c1nc(OC[C@@]23CCCN2C[C@H](F)C3)nc2c(F)c(-c3ccc(F)c4sc(N)nc34)c(C(F)(F)F)cc12] | LCMS m/z [M + 1]: 613.3 1H NMR (400 MHz, CD₃OD) δ 8.62 (s, 1H), 8.46 (s, 0.5H), 7.18 (t, 1H), 6.97 (t, 1H), 5.43 (s, 0.5H), 5.41 (s, 0.5H), 4.59-4.49 (m, 3H), 4.04-3.99 (m, 3H), 3.82-3.60 (m, 6H), 2.61-2.03 (m, 7H). |
| 82a | 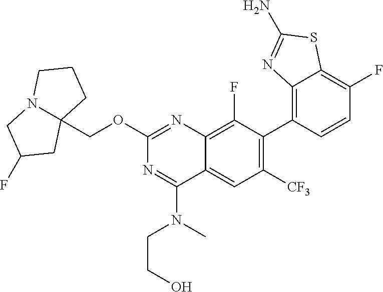 [SMILES: CN(CCO)c1nc(OCC23CCCN2CC(F)C3)nc2c(F)c(-c3ccc(F)c4sc(N)nc34)c(C(F)(F)F)cc12] | |
| 83 | 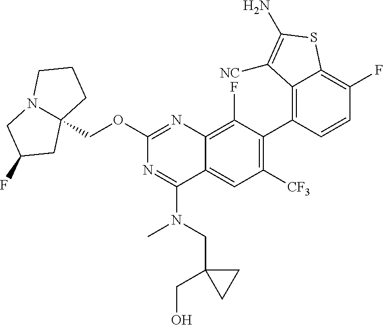 [SMILES: CN(CC1(CO)CC1)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 677 1H NMR (400 MHz, CD3OD) δ 8.45 (s, 1H), 7.18- 7.24 (m, 1H), 7.00 (t, 1H), 5.40 (s, 0.5H), 5.27 (s, 0.5H), 4.29-4.40 (m, 2H), 3.94-4.07 (m, 2H), 3.59 (s, 3H), 3.38-3.49 (m, 2H), 1.91-2.35 (m, 8H), 1.36 (d, 2H), 0.58-0.75 (m, 4H). |
| 83a | 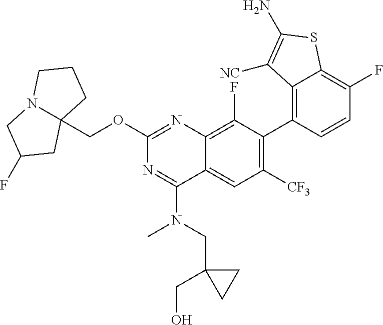 [SMILES: CN(CC1(CO)CC1)c1nc(OCC23CCCN2CC(F)C3)nc2c(F)c(-c3ccc(F)c4sc(N)c(C#N)c34)c(C(F)(F)F)cc12] | |
| 86 | 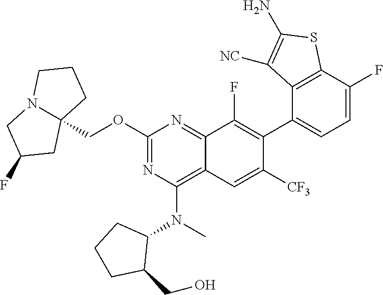 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@H]1CCC[C@@H]1CO] | LCMS m/z [M + 1]: 691.3 1H NMR (400 MHz, CD3OD) δ 8.35 (d, J = 6.4 Hz, 1H), 7.24-7.21 (m, 1H), 7.04-7.00 (m, 1H), 5.55 (d, J = 3.6 Hz, 0.54H), 5.42 (s, 0.53H), 4.98-4.93 (m, 1H), 4.65-4.49 (m, 2H), 3.88-3.53 (m, 5H), 3.42 (s, 3H), 2.60-1.63 (m, 14H), 1.61-1.52 (m, 1H). |
| 87 | 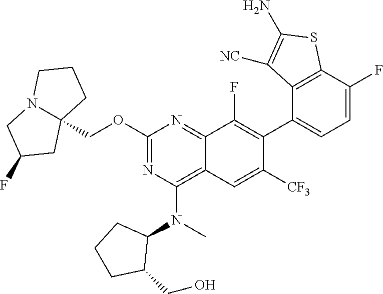 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@@H]1CCC[C@H]1CO] | LCMS m/z [M + 1]: 691.1 1H NMR (400 MHz, CD3OD) δ 8.33 (s, 1H), 7.20- 7.23 (m, 1H), 6.99-7.03 (t, 1H), 5.42 (m, 0.5H), 5.28 (m, 0.5H), 4.90-4.93 (m, 2H), 4.37 (s, 2H), 3.52-3.61 (m, 3H), 3.40 (s, 3H), 3.12 (m, 1H), 2.18-2.40 (m, 5H), 2.18-2.40 (m, 2H), 1.57-1.97-2.07 (m, 7H). |
| 87a | 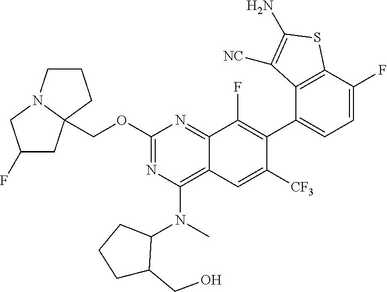 | |
| 88 | 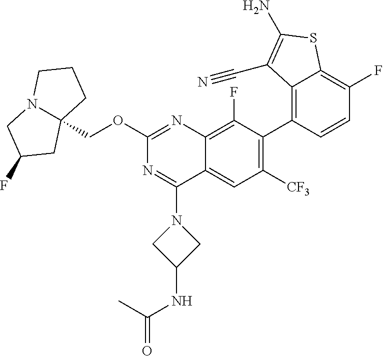 [SMILES: CC(=O)NC1CN(c2nc(OC[C@@]34CCCN3C[C@H](F)C4)nc3c(F)c(-c4ccc(F)c5sc(N)c(C#N)c45)c(C(F)(F)F)cc23)C1] | LCMS m/z [M + 1]: 675.7 1H NMR (400 MHz, CD3OD) δ 8.03 (s, 1H), 7.17- 7.21 (m, 1H), 6.98-7.02 (m, 1H), 5.37 (s, 0.5H), 5.24 (s, 0.5H), 4.77-4.82 (m, 2H), 4.49 (s, 2H), 4.21-4.33 (m, 2H), 3.21-3.26 (m, 3H), 2.99-3.05 (m, 1H), 2.07- 2.37 (m, 4H), 2.01 (s, 3H), 1.85-1.98 (m, 3H). |
| 88a | 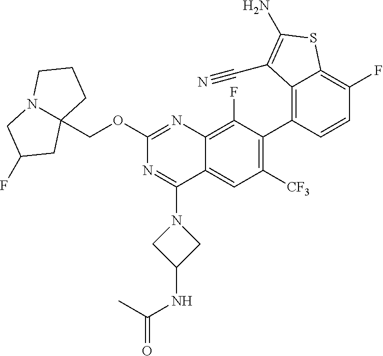 [SMILES: CC(=O)NC1CN(c2nc(OCC34CCCN3CC(F)C4)nc3c(F)c(-c4ccc(F)c5sc(N)c(C#N)c45)c(C(F)(F)F)cc23)C1] | |
| 89 | 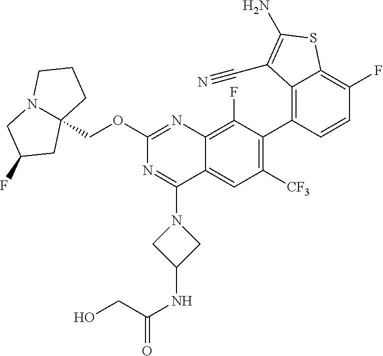 [SMILES: N#Cc1c(N)sc2c(F)ccc(-c3c(C(F)(F)F)cc4c(N5CC(NC(=O)CO)C5)nc(OC[C@@]56CCCN5C[C@H](F)C6)nc4c3F)c12] | LCMS m/z [M + 1]: 691.1 1HNMR (400 MHz, CD3OD) δ 8.08 (s, 1H), 7.15- 7.25 (m, 1H), 7.02 (t, 1H), 5.80 (s, 0.5H), 5.47 (s, 0.5H), 4.90-5.15 (m, 5H), 4.52-4.75 (m, 2H), 3.81- 4.09 (m, 4H), 3.41-3.48 (m, 1H), 2.52-2.72 (m, 2H), 2.21-2.49 (m, 3H) 1.92-2.20 (m, 2H). |
| 89a | 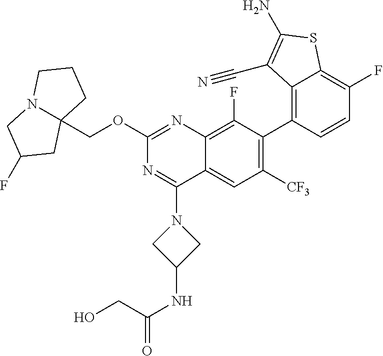 [SMILES: N#Cc1c(N)sc2c(F)ccc(-c3c(C(F)(F)F)cc4c(N5CC(NC(=O)CO)C5)nc(OCC56CCCN5CC(F)C6)nc4c3F)c12] | |
| 90 | 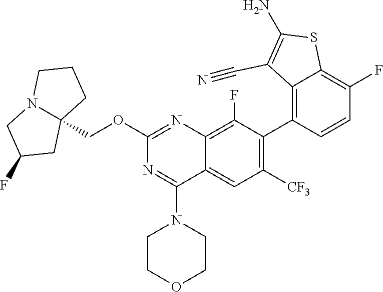 [SMILES: N#Cc1c(N)sc2c(F)ccc(-c3c(C(F)(F)F)cc4c(N5CCOCC5)nc(OC[C@@]56CCCN5C[C@H](F)C6)nc4c3F)c12] | LCMS m/z [M + 1]: 649 1H NMR (400 MHz, CD3OD) δ 8.19 (S, 1H), 7.20- 7.23 (m, 1H), 7.02 (t, 1H), 5.62 (s, 0.5H), 5.48 (s, 0.5H), 4.59-4.75 (m, 2H), 3.82-4.09 (m, 11H), 3.43- 3.47 (m, 1H), 2.15-2.72 (m, 6H). |
| 90a | 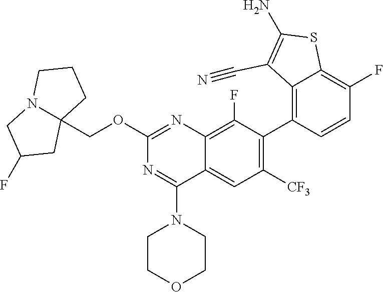 [SMILES: N#Cc1c(N)sc2c(F)ccc(-c3c(C(F)(F)F)cc4c(N5CCOCC5)nc(OCC56CCCN5CC(F)C6)nc4c3F)c12] | |
| 91 | 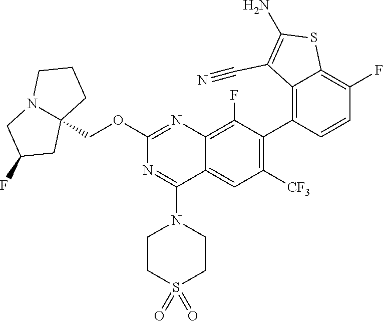 [SMILES: N#Cc1c(N)sc2c(F)ccc(-c3c(C(F)(F)F)cc4c(N5CCS(=O)(=O)CC5)nc(OC[C@@]56CCCN5C[C@H](F)C6)nc4c3F)c12] | LCMS m/z [M + 1]: 697.0 1H NMR (400 MHz, CD3OD) δ 8.25 (s, 1H), 7.23- 7.26 (m, 1H), 7.05 (t, 1H), 5.65 (s, 0.5H), 5.52 (s, 0.5H), 4.60-4.81 (m, 2H), 4.39-4.46 (m, 3H), 3.88- 4.08 (m, 3H), 3.41-3.58 (m, 4H), 2.33-2.79 (m, 8H). |
| 91a | 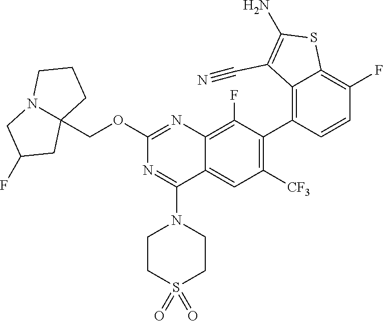 [SMILES: N#Cc1c(N)sc2c(F)ccc(-c3c(C(F)(F)F)cc4c(N5CCS(=O)(=O)CC5)nc(OCC56CCCN5CC(F)C6)nc4c3F)c12] | |
| 92 | 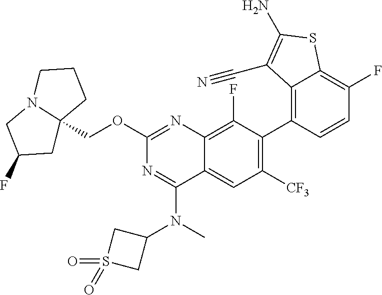 [SMILES: CN(c1nc(OC[C@@]23CCCN2C[C@H](F)C3)nc2c(F)c(-c3ccc(F)c4sc(N)c(C#N)c34)c(C(F)(F)F)cc12)C1CS(=O)(=O)C1] | |
| 92a | 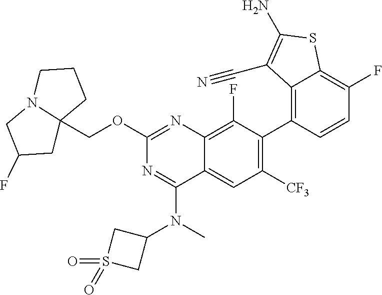 [SMILES: CN(c1nc(OCC23CCCN2CC(F)C3)nc2c(F)c(-c3ccc(F)c4sc(N)c(C#N)c34)c(C(F)(F)F)cc12)C1CS(=O)(=O)C1] | |
| 93 | 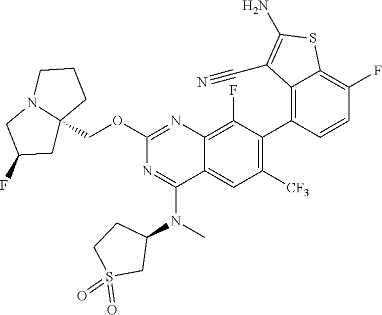 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@@H]1CCS(=O)(=O)C1] | LCMS m/z [M + 1]: 711.1 1H NMR (400 MHz, CD3OD) δ 8.36 (s, 1H), 7.26- 7.17 (m, 1H), 7.07-6.95 (m, 1H), 5.57-5.52 (m, 0.5H), 5.47-5.37 (m, 1.5H), 4.63-4.49 (m, 2H), 3.88- 3.61 (m, 4H), 3.60-3.49 (m, 3H), 3.49-3.37 (m, 2H), 3.28-3.16 (m, 2H), 2.75-1.96 (m, 9H). |
| 94 | 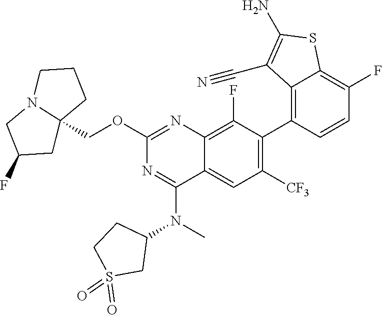 | LCMS m/z [M + 1]: 711.2 1H NMR (400 MHz, CD3OD) δ 8.34 (s, 1H), 7.23- 7.20 (m, 1H), 7.04-6.99 (m, 1H), 5.48-5.44 (m, 1.5H), 5.32 (s, 0.5H), 4.46-4.37 (m, 2H), 3.68-3.63 (m, 1H), 3.52 (s, 3H), 3.46-3.36 (m, 4H), 3.26-3.13 (m, 2H), 2.68-1.99 (m, 9H). |
| 94a | 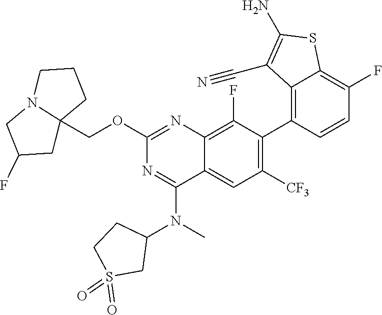 [SMILES: CN(c1nc(OCC23CCCN2CC(F)C3)nc2c(F)c(-c3ccc(F)c4sc(N)c(C#N)c34)c(C(F)(F)F)cc12)C1CCS(=O)(=O)C1] | |
| 95 | 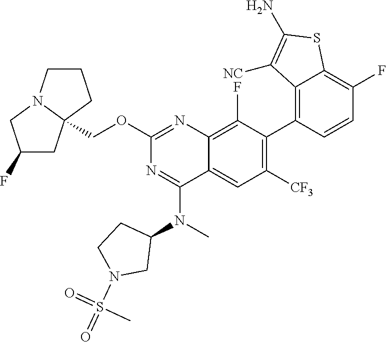 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@@H]1CCN(S(C)(=O)=O)C1] | LCMS m/z [M + 1]: 741.3 1H NMR (400 MHz, CD3OD) δ 8.49 (s, 0.5H), 8.32 (s, 1H), 7.20-7.23 (m, 1H), 7.01 (t, 1H), 5.48 (s, 0.5H), 5.31 (s, 0.5H), 5.26-5.29 (m, 1H), 4.40-4.59 (m, 2H), 3.79-3.82 (m, 1H), 3.63 (s, 2H), 3.46-3.49 (m, 7H), 3.37-3.46 (m, 1H), 2.97 (s, 3H), 2.38-2.49 (m, 4H), 2.24-2.29 (m, 1H), 2.03-2.14 (m, 2H), 1.95- 2.03 (m, 1H). |
| 95a | 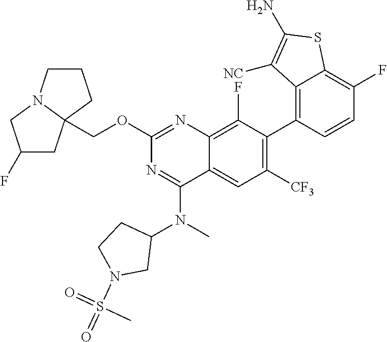 [SMILES: CN(c1nc(OCC23CCCN2CC(F)C3)nc2c(F)c(-c3ccc(F)c4sc(N)c(C#N)c34)c(C(F)(F)F)cc12)C1CCN(S(C)(=O)=O)C1] | |
| 96 | 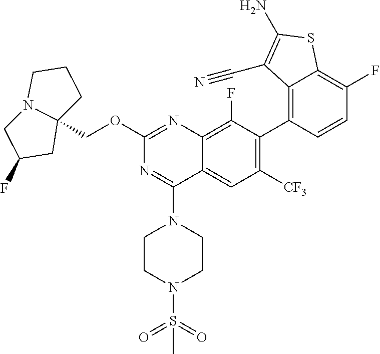 [SMILES: CS(=O)(=O)N1CCN(c2nc(OC[C@@]34CCCN3C[C@H](F)C4)nc3c(F)c(-c4ccc(F)c5sc(N)c(C#N)c45)c(C(F)(F)F)cc23)CC1] | LCMS m/z [M + 1]: 726.1 1H NMR (400 MHz, CD3OD) δ 8.17 (s, 1H), 7.25- 7.20 (m, 1H), 7.04-7.00 (m, 1H), 5.51 (s, 0.61H), 5.38 (s, 0.51H), 4.56-4.44 (m, 2H), 4.11-4.09 (m, 3H), 3.76-3.58 (m, 3H), 3.48-3.46 (m, 3H), 3.30-3.23 (m, 2H), 2.91 (s, 3H), 2.58-2.03 (m, 7H). |
| 96a | 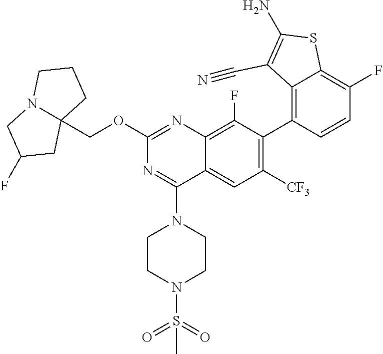 [SMILES: CS(=O)(=O)N1CCN(c2nc(OCC34CCCN3CC(F)C4)nc3c(F)c(-c4ccc(F)c5sc(N)c(C#N)c45)c(C(F)(F)F)cc23)CC1] | |
| 97 | 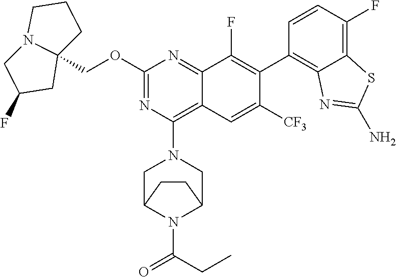 [SMILES: CCC(=O)N1C2CCC1CN(c1nc(OC[C@@]34CCCN3C[C@H](F)C4)nc3c(F)c(-c4ccc(F)c5sc(N)nc45)c(C(F)(F)F)cc13)C2] | LCMS m/z [M + 1]: 706.3 1H NMR (400 MHz, CD3OD): δ 8.55 (s, 1H), 7.25- 7.17 (m, 1H), 7.00 (t, J = 8.8 Hz, 1H), 5.56 (d, J = 51.6 Hz, 1H), 4.80-4.50 (m, 6H), 4.10-3.80 (m, 4H), 3.78-3.68 (m, 1H), 3.52-3.42 (m, 1H), 2.80-2.30 (m, 7H), 2.25-2.00 (m, 2H), 2.00-1.75 (m, 3H), 1.20 (t, J = 7.2 Hz, 3H). |
| 97a | 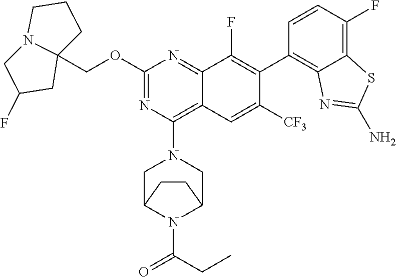 [SMILES: CCC(=O)N1C2CCC1CN(c1nc(OCC34CCCN3CC(F)C4)nc3c(F)c(-c4ccc(F)c5sc(N)nc45)c(C(F)(F)F)cc13)C2] | |
| 98 | 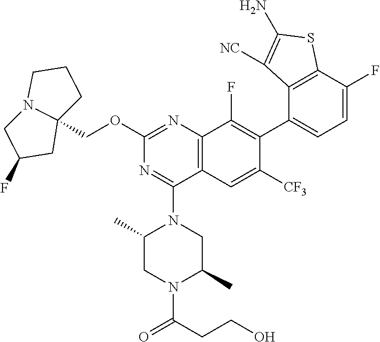 | LCMS m/z [M + 2]/2: 376.4 1H NMR (400 MHz, CD3OD): δ 8.23 (s, 1H), 7.25- 7.20 (m, 1H), 7.04 (t, J = 8.8 Hz, 1H), 5.58 (d, J = 52 Hz, 1H), 5.00-4.90 (m, 1H), 4.80-4.70 (m, 2H), 4.50- 4.25 (br s, 1H), 4.23-4.12 (m, 1 H), 4.10-3.85 (m, 4H), 3.78-3.71 (m, 1H), 3.70-3.58 (m, 1H), 3.55-3.45 (m, 3H), 2.95-2.85 (m, 2H), 2.80-2.60 (m, 2H), 2.50- 2.35 (m, 3H), 2.30-2.10 (m, 1H), 1.66-1.58 (m, 3H), 1.50-1.35 (m, 3H). |
| 98a | 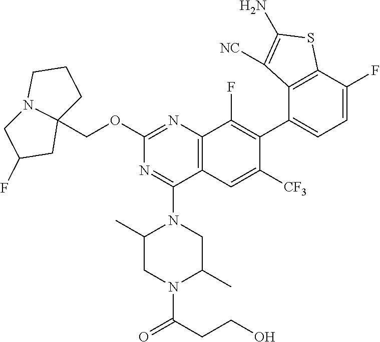 [SMILES: CC1CN(c2nc(OCC34CCCN3CC(F)C4)nc3c(F)c(-c4ccc(F)c5sc(N)c(C#N)c45)c(C(F)(F)F)cc23)C(C)CN1C(=O)CCO] | |
| 99 | 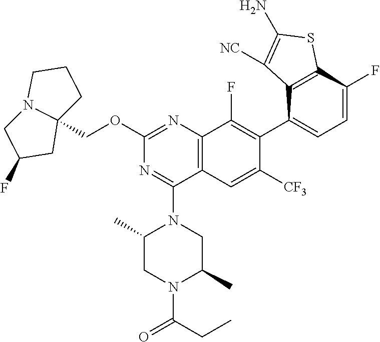 | LCMS m/z [M + 1]: 732.0 1H NMR (400 MHz, CD3OD): δ 8.19 (s, 1H), 7.30- 7.23 (m, 1H), 7.03 (t, J = 8.8 Hz, 1H), 5.60 (d, J = 59 Hz, 1H), 5.00-4.85 (m, 1H), 4.80-4.63 (m, 2H), 4.45- 4.30 (m, 2H), 4.10-3.75 (m, 5H), 3.53-3.45 (m, 2H), 2.75-2.00 (m, 8H), 1.60-1.45 (m, 3H), 1.40-1.32 (m, 3H), 1.30-1.12 (m, 3H). |
| 99a | 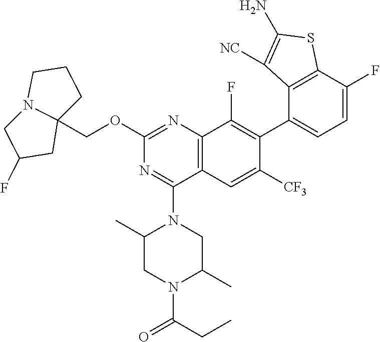 [SMILES: CCC(=O)N1CC(C)N(c2nc(OCC34CCCN3CC(F)C4)nc3c(F)c(-c4ccc(F)c5sc(N)c(C#N)c45)c(C(F)(F)F)cc23)CC1C] | |
| 100 | 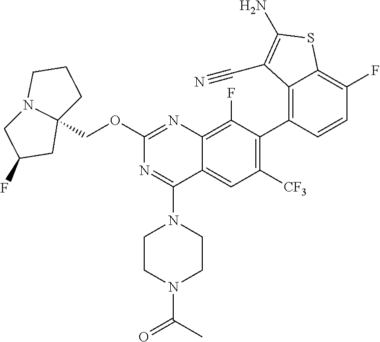 [SMILES: CC(=O)N1CCN(c2nc(OC[C@@]34CCCN3C[C@H](F)C4)nc3c(F)c(-c4ccc(F)c5sc(N)c(C#N)c45)c(C(F)(F)F)cc23)CC1] | LCMS m/z [M + 1]: 690.0 1H NMR (400 MHz, CD3OD) δ 8.54 (s, 1H), 7.23- 7.19 (m, 1H), 7.03-6.99 (m, 1H), 5.37 (s, 0.42H), 5.23 (s, 0.64H), 4.34-4.23 (m, 2H), 4.08-3.99 (m, 4H), 3.82 (d, J = 10 Hz, 4H), 3.25-3.20 (m, 3H), 3.05- 2.99 (m, 1H), 2.36-2.21 (m, 2H), 2.17 (s, 3H), 2.03- 1.88 (m, 4H), 1.30 (s, 2H). |
| 100a | 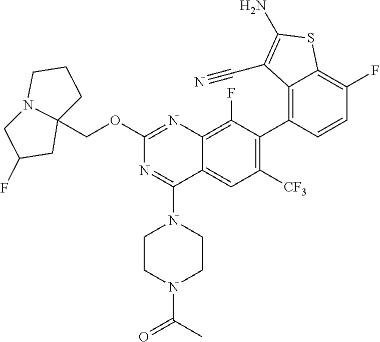 [SMILES: CC(=O)N1CCN(c2nc(OCC34CCCN3CC(F)C4)nc3c(F)c(-c4ccc(F)c5sc(N)c(C#N)c45)c(C(F)(F)F)cc23)CC1] | |
| 101 | 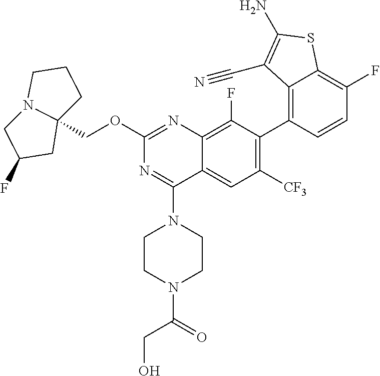 [SMILES: N#Cc1c(N)sc2c(F)ccc(-c3c(C(F)(F)F)cc4c(N5CCN(C(=O)CO)CC5)nc(OC[C@@]56CCCN5C[C@H](F)C6)nc4c3F)c12] | LCMS m/z [M + 1]: 706.1 1H NMR (400 MHz, CD3OD) δ 8.21 (s, 1H), 7.22 (s, 1H), 7.05-7.00 (s, 1H), 5.49 (s, 0.42H), 5.35 (s, 0.54H), 4.51-4.41 (m, 2H), 4.31 (s, 2H), 4.09 (s, 5H), 3.86 (s, 2H), 3.72 (s, 3H), 3.54 (s, 3H), 2.50-2.04 (m, 6H). |
| 101a | 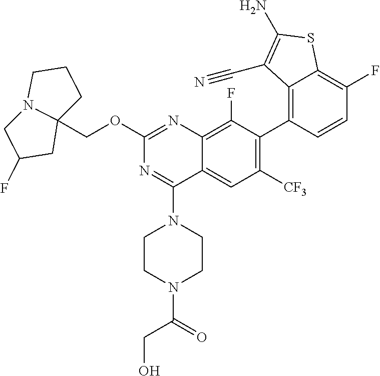 [SMILES: N#Cc1c(N)sc2c(F)ccc(-c3c(C(F)(F)F)cc4c(N5CCN(C(=O)CO)CC5)nc(OCC56CCCN5CC(F)C6)nc4c3F)c12] | |
| 102 | 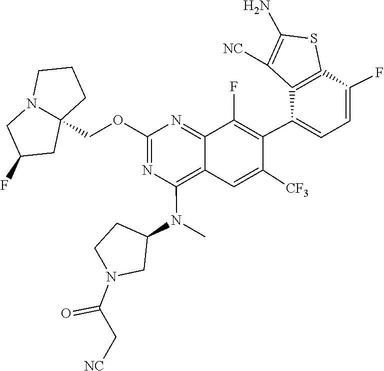 | LCMS m/z [M + 1]: 729.3 1H NMR (400 MHz, CD3OD): δ 8.37 (s, 1H), 7.28- 7.22 (m, 1H), 7.03 (t, J = 8.8 Hz, 1H), 5.60 (d, J = 59 Hz, 1H), 5.42-5.22 (m, 1H), 4.80-4.60 (m, 2H), 4.15- 3.80 (m, 7H), 3.70-3.62 (m, 1H), 3.60-3.40 (m, 5H), 2.80-2.30 (m, 7H), 2.26-2.12 (m, 1H). |
| 103 | 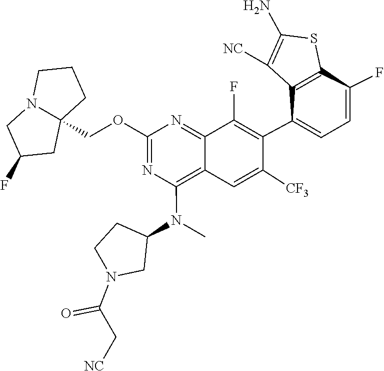 | LCMS m/z [M + 1]: 729.3 1H NMR (400 MHz, CD3OD): δ 8.37 (s, 1H), 7.28- 7.22 (m, 1H), 7.03 (t, J = 8.8 Hz, 1H), 5.60 (d, J = 59 Hz, 1H), 5.45-5.25 (m, 1H), 4.80-4.60 (m, 2H), 4.30- 4.17 (m, 2H), 4.10-3.75 (m, 5H), 3.70-3.40 (m, 6H), 2.80-2.30 (m, 7H), 2.22-2.10 (m, 1H). |
| 103a | 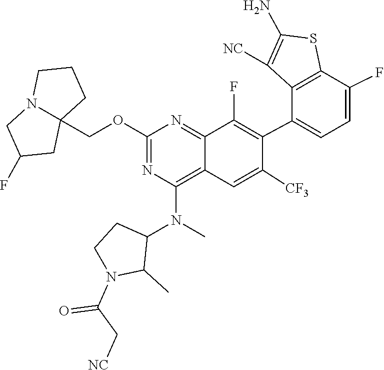 [SMILES: CC1C(N(C)c2nc(OCC34CCCN3CC(F)C4)nc3c(F)c(-c4ccc(F)c5sc(N)c(C#N)c45)c(C(F)(F)F)cc23)CCN1C(=O)CC#N] | |
| 104 | 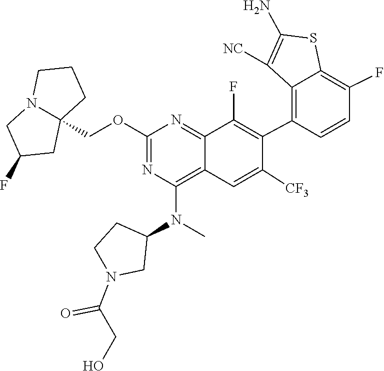 | LCMS m/z [M + 1]: 729.3 1H NMR (400 MHz, CD3OD): δ 8.37 (s, 1H), 7.28- 7.22 (m, 1H), 7.03 (t, J = 8.8 Hz, 1H), 5.60 (d, J = 59 Hz, 1H), 5.45-5.25 (m, 1H), 4.80-4.60 (m, 2H), 4.30- 4.17 (m, 2H), 4.10-3.75 (m, 5H), 3.70-3.40 (m, 6H), 2.80-2.30 (m, 7H), 2.22-2.10 (m, 1H). |
| 104a | 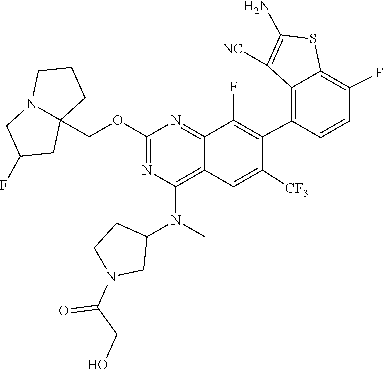 [SMILES: CN(c1nc(OCC23CCCN2CC(F)C3)nc2c(F)c(-c3ccc(F)c4sc(N)c(C#N)c34)c(C(F)(F)F)cc12)C1CCN(C(=O)CO)C1] | |
| 105 | 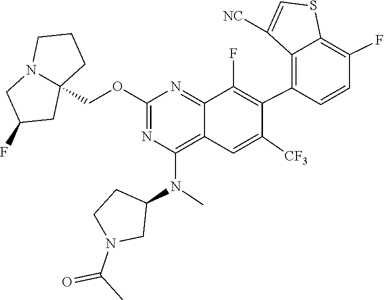 [SMILES: CC(=O)N1CC[C@@H](N(C)c2nc(OC[C@@]34CCCN3C[C@H](F)C4)nc3c(F)c(-c4ccc(F)c5scc(C#N)c45)c(C(F)(F)F)cc23)C1] | LCMS m/z [M + 1]: 689.4 1H NMR (400 MHz, CD3OD): δ 8.70 (s, 1H), 8.42 (d, J = 4.8 Hz, 1H), 7.57 (dd, J = 8.0, 4.8 Hz, 1H), 7.47 (t, J = 8.8 Hz, 1H), 5.56 (d, J = 51.2 Hz, 1H), 5.50-5.28 (m, 1H), 4.80-4.60 (m, 2H), 4.10-3.80 (m, 4H), 3.78-3.60 (m, 1H), 3.58-3.40 (m, 5H), 2.75-2.30 (m, 7H), 2.25-2.10 (m, 4H). |
| 105a | 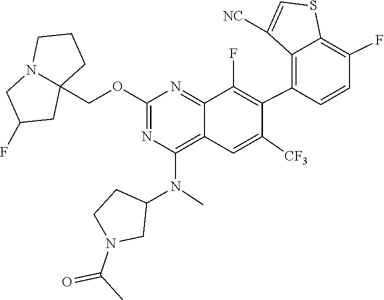 [SMILES: CC(=O)N1CCC(N(C)c2nc(OCC34CCCN3CC(F)C4)nc3c(F)c(-c4ccc(F)c5scc(C#N)c45)c(C(F)(F)F)cc23)C1] | |
| 106 | 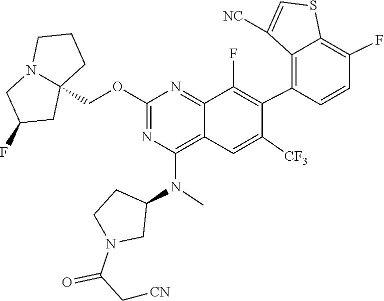 [SMILES: CN(c1nc(OC[C@@]23CCCN2C[C@H](F)C3)nc2c(F)c(-c3ccc(F)c4scc(C#N)c34)c(C(F)(F)F)cc12)[C@@H]1CCN(C(=O)CC#N)C1] | LCMS m/z [M + 1]: 714.2 1H NMR (400 MHz, CD3OD): δ 8.70 (s, 1H), 8.42 (s, 1H), 7.62-7.55 (m, 1H), 7.46 (t, J = 8.8 Hz, 1H), 5.54 (d, J = 56.4 Hz, 1H), 5.45-5.25 (m, 1H), 4.75- 4.60 (m, 2H), 4.15-4.05 (m, 2H), 4.05-3.80 (m, 4H), 3.75-3.65 (m, 1H), 3.65-3.40 (m, 4H), 2.55-2.30 (m, 7H), 2.25-2.10 (m, 1H). |
| 106a | 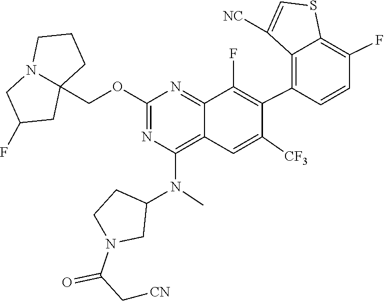 [SMILES: CN(c1nc(OCC23CCCN2CC(F)C3)nc2c(F)c(-c3ccc(F)c4scc(C#N)c34)c(C(F)(F)F)cc12)C1CCN(C(=O)CC#N)C1] | |
| 107 | 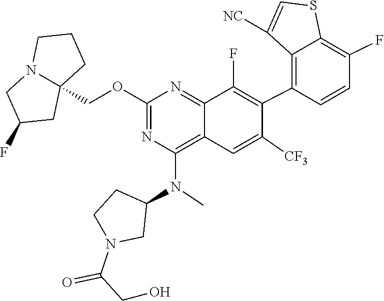 [SMILES: CN(c1nc(OC[C@@]23CCCN2C[C@H](F)C3)nc2c(F)c(-c3ccc(F)c4scc(C#N)c34)c(C(F)(F)F)cc12)[C@@H]1CCN(C(=O)CO)C1] | LCMS m/z [M + 1]: 705.2 1H NMR (400 MHz, CD3OD): δ 8.70 (s, 1H), 8.42 (s, 1H), 7.62-7.55 (m, 1H), 7.47 (t, J = 8.8 Hz, 1H), 5.54 (d, J = 57.6 Hz, 1H), 5.42-5.22 (m, 1H), 4.80- 4.60 (m, 3H), 4.35-4.20 (m, 2H), 4.15-3.70 (m, 5H), 3.65-3.40 (m, 5H), 2.80-2.00 (m, 8H). |
| 107a | 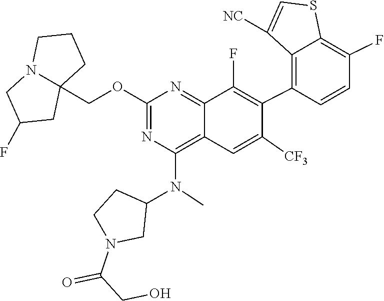 [SMILES: CN(c1nc(OCC23CCCN2CC(F)C3)nc2c(F)c(-c3ccc(F)c4scc(C#N)c34)c(C(F)(F)F)cc12)C1CCN(C(=O)CO)C1] | |
| 112 | 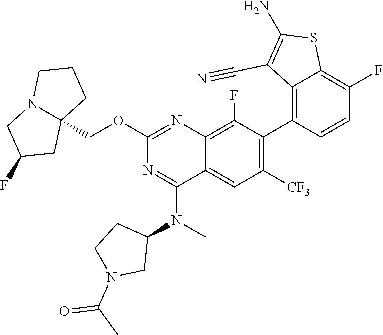 [SMILES: CC(=O)N1CC[C@@H](N(C)c2nc(OC[C@@]34CCCN3C[C@H](F)C4)nc3c(F)c(-c4ccc(F)c5sc(N)c(C#N)c45)c(C(F)(F)F)cc23)C1] | LCMS m/z [M + 1]: 704.1 1H NMR (400 MHz, CD3OD) δ 8.35 (s, 1H), 7.25- 7.21 (m, 1H), 7.05-7.00 (m, 1H), 5.52-5.49 (m, 3H), 5.40-5.19 (m, 2H), 4.55-4.44 (m, 2H), 4.08-3.99 (m, 1H), 3.89-3.81 (m, 1H), 3.72-3.62 (m, 4H), 3.55- 3.42 (m, 3H), 2.76-2.33 (m, 5H), 2.30-2.04 (m, 6H). |
| 112a | 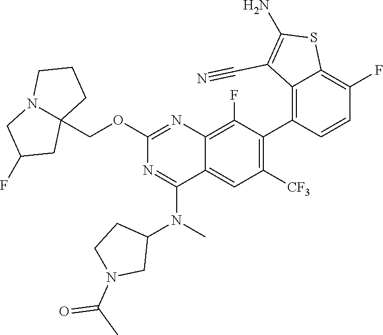 [SMILES: CC(=O)N1CCC(N(C)c2nc(OCC34CCCN3CC(F)C4)nc3c(F)c(-c4ccc(F)c5sc(N)c(C#N)c45)c(C(F)(F)F)cc23)C1] | |
| 113 | 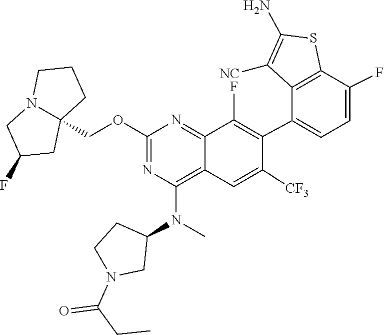 [SMILES: CCC(=O)N1CC[C@@H](N(C)c2nc(OC[C@@]34CCCN3C[C@H](F)C4)nc3c(F)c(-c4ccc(F)c5sc(N)c(C#N)c45)c(C(F)(F)F)cc23)C1] | LCMS m/z [M + 1]: 718.4 1H NMR (400 MHz, CD3OD) δ 8.33 (s, 1H), 7.24- 7.21 (m, 1H), 7.04-7.00 (m, 1H), 5.49-5.19 (m, 3H), 4.61-4.31 (m, 2H), 4.09-3.99 (m, 2H), 3.86- 3.83(m, 1H), 3.71-3.62 (m, 1H), 3.59-3.35 (m, 6H), 3.16-3.13 (m, 1H), 2.49-1.95 (m, 10H), 1.18-1.14 (m, 3H). |
| 113a | 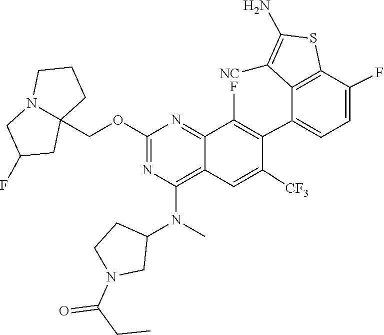 [SMILES: CCC(=O)N1CCC(N(C)c2nc(OCC34CCCN3CC(F)C4)nc3c(F)c(-c4ccc(F)c5sc(N)c(C#N)c45)c(C(F)(F)F)cc23)C1] | |
| 114 | 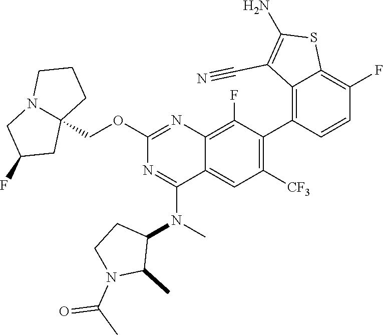 | LCMS m/z [M + 1]: 718.2 1H NMR (400 MHz, CD3OD) δ 8.39 (s, 1H), 7.25 (t, 1H), 7.04 (t, 1H), 5.53-5.53 (m, 1H), 5.02-4.98 (m, 1H), 4.62-4.44 (m, 2H), 3.65-3.62 (m, 6H), 3.53-3.50 (m, 1H), 3.25-3.19 (m, 2H), 2.48-2.43 (m, 2H), 2.17 (s, 2H), 2.11 (s, 3H), 1.33 (m, 5H), 1.18-1.17 (m, 1H), 1.11-1.07 (m, 2H). |
| 114a | 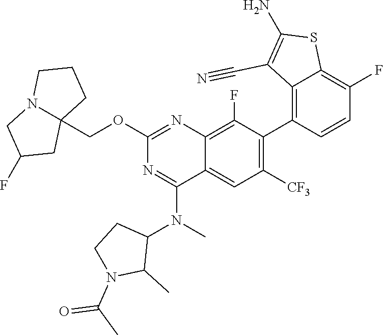 [SMILES: CC(=O)N1CCC(N(C)c2nc(OCC34CCCN3CC(F)C4)nc3c(F)c(-c4ccc(F)c5sc(N)c(C#N)c45)c(C(F)(F)F)cc23)C1C] | |
| 115 | 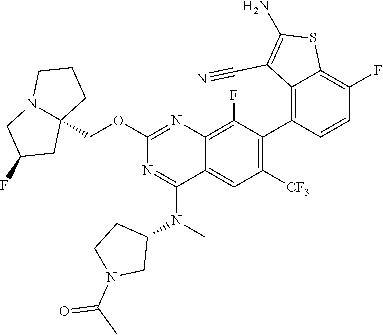 [SMILES: CC(=O)N1CC[C@H](N(C)c2nc(OC[C@@]34CCCN3C[C@H](F)C4)nc3c(F)c(-c4ccc(F)c5sc(N)c(C#N)c45)c(C(F)(F)F)cc23)C1] | LCMS m/z [M + 1]: 704.3 1H NMR (400 MHz, CD3OD) δ 8.31 (s, 1H), 7.17- 7.25 (m, 1H), 7.01 (t, 1H), 5.22-5.40 (m, 2H), 4.20- 4.35 (m, 2H), 3.95-4.10 (m, 1H), 3.77-3.87 (m, 1H), 3.47-3.72 (m, 2H), 3.40-3.45 (m, 3H), 3.17-3.27 (m, 2H), 2.98-3.08 (m, 1H), 2.20-2.50 (m, 4H), 2.13-2.20 (m, 1H), 2.05-2.13 (d, 3H), 1.75-2.05 (m, 4H). |
| 115a | 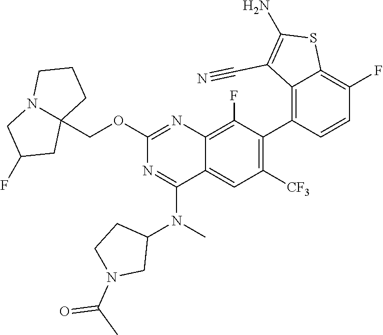 [SMILES: CC(=O)N1CCC(N(C)c2nc(OCC34CCCN3CC(F)C4)nc3c(F)c(-c4ccc(F)c5sc(N)c(C#N)c45)c(C(F)(F)F)cc23)C1] | |
| 116 | 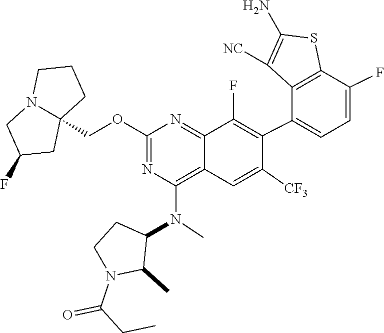 [SMILES: CCC(=O)N1CC[C@@H](N(C)c2nc(OC[C@@]34CCCN3C[C@H](F)C4)nc3c(F)c(-c4ccc(F)c5sc(N)c(C#N)c45)c(C(F)(F)F)cc23)[C@H]1C] | LCMS m/z [M + 1]: 732.2 1H NMR (400 MHz, CD3OD) δ 8.37 (s, 1H), 7.23- 7.20 (m, 1H), 7.02 (t, 1H), 5.50-5.33 (m, 1H), 4.99- 4.95 (m, 1H), 4.56-4.41 (m, 2H), 3.62-3.43 (m, 7H), 2.46-2.34 (m, 4H), 2.23-2.03 (m, 5H), 1.28 (s, 4H), 1.19-1.04 (m, 6H). |
| 116a | 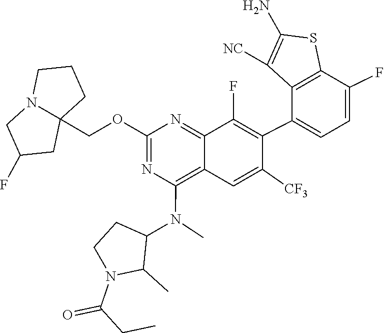 [SMILES: CCC(=O)N1CCC(N(C)c2nc(OCC34CCCN3CC(F)C4)nc3c(F)c(-c4ccc(F)c5sc(N)c(C#N)c45)c(C(F)(F)F)cc23)C1C] | |
| 117 | 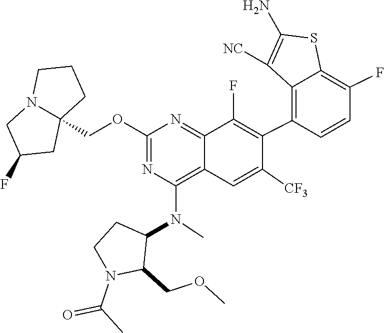 [SMILES: COC[C@@H]1[C@H](N(C)c2nc(OC[C@@]34CCCN3C[C@H](F)C4)nc3c(F)c(-c4ccc(F)c5sc(N)c(C#N)c45)c(C(F)(F)F)cc23)CCN1C(C)=O] | LCMS m/z [M + 1]: 748.2 1H NMR (400 MHz, CD3OD) δ 8.37(d, 1H), 7.23- 7.20 (m, 1H), 7.01 (t, 1H), 5.37 (s, 0.5H), 5.24 (s, 0.5H), 4.99-4.92 (m, 1H), 4.36-4.17 (m, 2H), 3.81- 3.61 (m, 6H), 3.45-3.33 (m, 1H), 3.29-3.15 (m, 6H), 3.03 (t, 1H), 2.85-2.74 (m, 1H), 2.35-1.89 (m, 11H). |
| 117a | 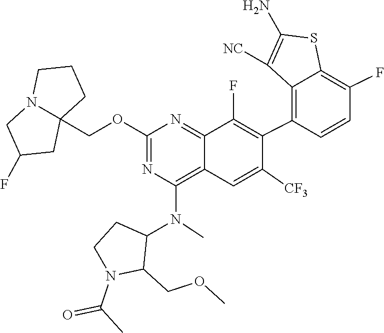 [SMILES: COCC1C(N(C)c2nc(OCC34CCCN3CC(F)C4)nc3c(F)c(-c4ccc(F)c5sc(N)c(C#N)c45)c(C(F)(F)F)cc23)CCN1C(C)=O] | |
| 118 | 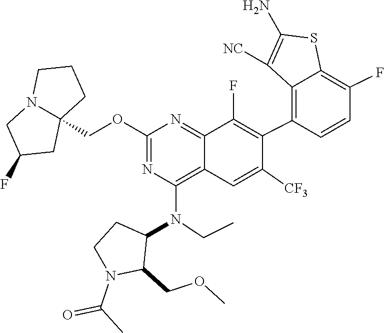 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@@H]1CCN(C(C)=O)[C@@H]1COC] | LCMS m/z [M + 1]: 762.2 1H NMR (400 MHz, CD3OD) δ 8.36 (s, 1H), 7.18- 7.28 (m, 1H), 7.01 (t, 1H), 5.37 (s, 0.5H), 5.24 (s, 0.5H), 4.87-4.95 (m, 2H), 4.19-4.39 (m, 2H), 3.97- 4.14 (m, 2H), 3.78 (t, 1H), 3.54-3.72 (m, 2H), 3.14- 3.23 (m, 3H), 2.95-3.10 (m, 2H), 2.68-2.94 (m, 3H), 2.54-2.67 (m, 1H), 2.19-2.49 (m, 4H), 2.14-2.19 (m, 1H), 2.10 (s, 2H), 1.88-2.07 (m, 3H), 1.35-1.53 (m, 3H). |
| 118a | 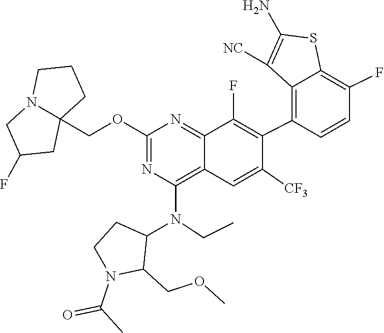 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(C)=O)C1COC] | |
| 119 | 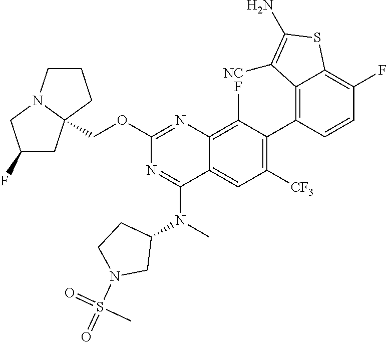 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@H]1CCN(S(C)(=O)=O)C1] | LCMS m/z [M + 1]: 740.3 1H NMR (400 MHz, CD3OD) δ 8.30 (s, 1H), 7.17- 7.27 (m, 1H), 7.01 (t, 1H), 5.22-5.42 (m, 4H), 4.59 (s, 3H), 4.27-4.38 (m, 1H), 3.77-3.87 (m, 1H), 3.58- 3.68 (m, 1H), 3.33-3.53 (m, 4H), 3.03-3.12 (m, 1H), 2.97 (s, 1H), 2.35-2.53 (m, 2H), 2.23-2.33 (m, 1H), 2.18 (t, 2H), 1.97-2.07 (m, 3H), 1.52-1.65 (m, 2H). |
| 119a | 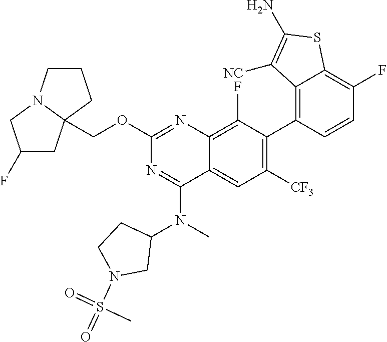 [SMILES: CN(c1nc(OCC23CCCN2CC(F)C3)nc2c(F)c(-c3ccc(F)c4sc(N)c(C#N)c34)c(C(F)(F)F)cc12)C1CCN(S(C)(=O)=O)C1] | |
| 120 | 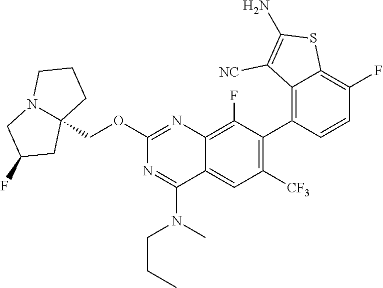 [SMILES: CCCN(C)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 635 1H NMR (400 MHz, CD3OD) δ 8.31 (s, 1H), 7.16- 7.26 (m, 1H), 7.01 (t, 1H), 5.51 (s, 0.5H), 5.38 (s, 0.5H), 4.38-4.61 (m, 2H), 3.77-3.90 (m, 2H), 3.59- 3.76 (m, 2H), 3.53 (s, 3H), 3.31-3.34 (m, 1H), 3.25- 3.29 (m, 1H), 1.99-2.63 (m, 6H), 1.83-1.99 (m, 2H), 1.05 (t, 3H). |
| 120a | 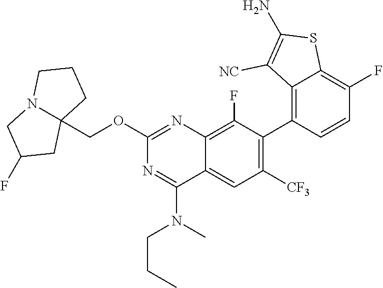 [SMILES: CCCN(C)c1nc(OCC23CCCN2CC(F)C3)nc2c(F)c(-c3ccc(F)c4sc(N)c(C#N)c34)c(C(F)(F)F)cc12] | |
| 121 | 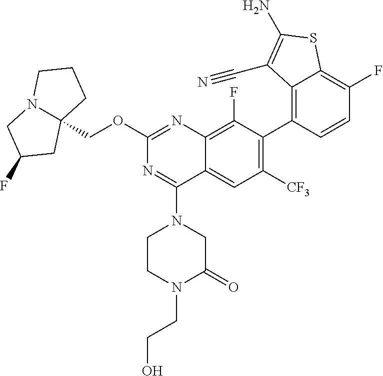 [SMILES: N#Cc1c(N)sc2c(F)ccc(-c3c(C(F)(F)F)cc4c(N5CCN(CCO)C(=O)C5)nc(OC[C@@]56CCCN5C[C@H](F)C6)nc4c3F)c12] | LCMS m/z [M + 1]: 706.3 1H NMR (400 MHz, CD3OD) δ 8.23 (s, 1H), 7.30- 7.14 (m, 1H), 7.09-6.93 (m, 1H), 5.45 (s, 0.5H), 5.31 (s, 0.5H), 4.65 (s, 1H), 4.53-4.16 (m, 3H), 3.85-3.69 (m, 2H), 3.66-3.34 (m, 3H), 3.29-3.07 (m, 5H), 2.95- 2.65 (m, 2H), 2.52-1.80 (m, 6H). |
| 121a | 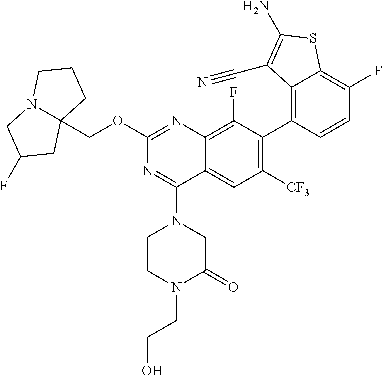 [SMILES: N#Cc1c(N)sc2c(F)ccc(-c3c(C(F)(F)F)cc4c(N5CCN(CCO)C(=O)C5)nc(OCC56CCCN5CC(F)C6)nc4c3F)c12] | |
| 122 | 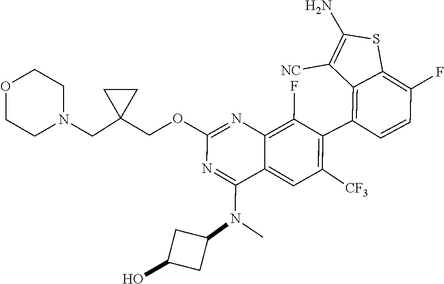 | LCMS m/z [M + 1]: 675.0 1H NMR (400 MHz, CD3OD) δ 8.19 (s, 1H), 7.19- 7.23 (m, 1H), 6.99-7.03 (t, J = 8.8 Hz, 1H), 4.43-4.44 (m, 2H), 4.30-4.33 (m, 1H), 4.05-4.09 (m, 1H), 3.69- 3.71 (m, 3H), 3.41 (s, 3H), 2.61-2.87 (m, 8H), 2.24- 2.27 (m, 2H), 1.29-1.33 (m, 3H), 0.74-0.77 (m, 2H), 0.55-0.57 (m, 2H). |
| 122a | 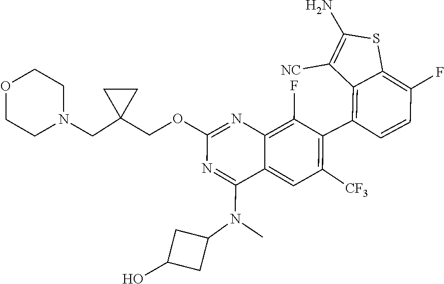 [SMILES: CN(c1nc(OCC2(CN3CCOCC3)CC2)nc2c(F)c(-c3ccc(F)c4sc(N)c(C#N)c34)c(C(F)(F)F)cc12)C1CC(O)C1] | |
| 123 | 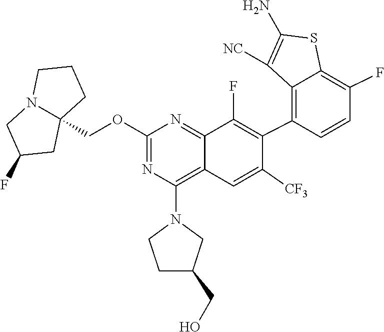 [SMILES: N#Cc1c(N)sc2c(F)ccc(-c3c(C(F)(F)F)cc4c(N5CC[C@H](CO)C5)nc(OC[C@@]56CCCN5C[C@H](F)C6)nc4c3F)c12] | LCMS m/z [M + 1]: 663.1 1H NMR (400 MHz, CD3OD) δ 8.49 (s, 1H), 7.23- 7.20 (m, 1H), 7.04-7.00 (m, 1H), 5.59 (d, J = 3.6 Hz, 0.52H), 5.46 (d, J = 3.6 Hz, 0.53H), 4.71-4.55 (m, 2H), 4.17-3.62 (m, 9H), 3.48-3.37 (m, 1H), 2.71-1.91 (m, 9H). |
| 124 | 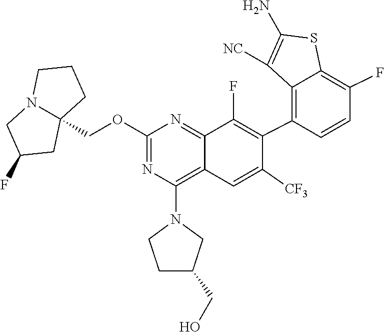 [SMILES: N#Cc1c(N)sc2c(F)ccc(-c3c(C(F)(F)F)cc4c(N5CC[C@@H](CO)C5)nc(OC[C@@]56CCCN5C[C@H](F)C6)nc4c3F)c12] | LCMS m/z [M + 1]: 663.19 1H NMR (400 MHz, CD3OD) δ 8.45 (s, 1H), 7.24- 7.17 (m, 1H), 7.04-6.95 (m, 1H), 5.39 (s, 0.5H), 5.26 (s, 0.5H), 4.40-4.25 (m, 2H), 4.21-3.99 (m, 3H), 3.88-3.79 (m, 1H), 3.74-3.60 (m, 2H), 3.49-3.38 (m, 1H), 3.14-2.99 (m, 2H), 2.68-2.51 (m, 2H), 2.45-1.76 (m, 10H). |
| 124a | 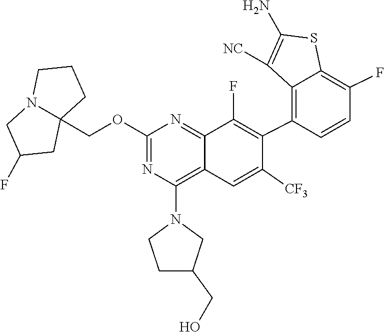 [SMILES: N#Cc1c(N)sc2c(F)ccc(-c3c(C(F)(F)F)cc4c(N5CCC(CO)C5)nc(OCC56CCCN5CC(F)C6)nc4c3F)c12] | |
| 125 | 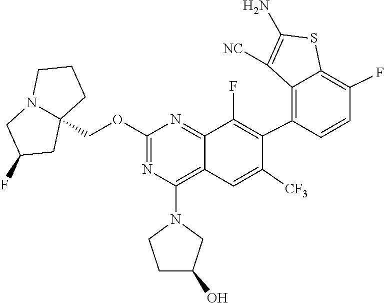 [SMILES: N#Cc1c(N)sc2c(F)ccc(-c3c(C(F)(F)F)cc4c(N5CC[C@H](O)C5)nc(OC[C@@]56CCCN5C[C@H](F)C6)nc4c3F)c12] | LCMS m/z [M + 1]: 649.0 1H NMR (400 MHz, CD3OD) δ 8.47 (s, 1H), 7.23- 7.20 (m, 1H), 7.04-6.99 (m, 1H), 5.52 (s, 0.47H), 5.38 (s, 0.50H), 4.61-4.46 (m, 3H), 4.14-3.56 (m, 7H), 3.28-3.13 (m, 2H), 2.48-2.02 (m, 8H). |
| 126 | 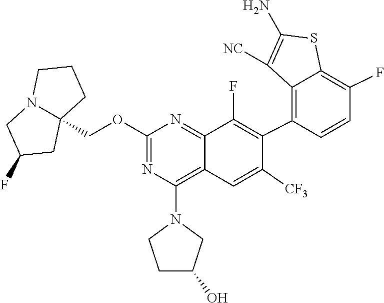 [SMILES: N#Cc1c(N)sc2c(F)ccc(-c3c(C(F)(F)F)cc4c(N5CC[C@@H](O)C5)nc(OC[C@@]56CCCN5C[C@H](F)C6)nc4c3F)c12] | LCMS m/z [M + 1]: 649.17 1H NMR (400 MHz, CD3OD) δ 8.44 (s, 1H), 7.24- 7.17 (m, 1H), 7.03-6.97 (m, 1H), 5.39 (s, 0.5H), 5.25 (s, 0.5H), 4.60 (s, 1H), 4.41-4.35 (m, 1H), 4.28-4.06 (m, 4H), 3.97-3.91 (m, 1H), 3.49-3.37 (m, 1H), 3.14- 3.00 (m, 2H), 2.42-1.85 (m, 10H). |
| 126a | 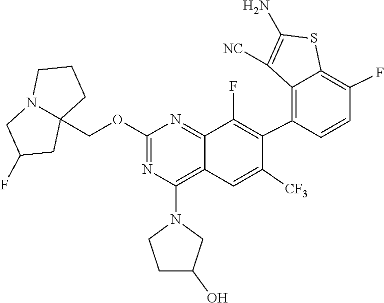 [SMILES: N#Cc1c(N)sc2c(F)ccc(-c3c(C(F)(F)F)cc4c(N5CCC(O)C5)nc(OCC56CCCN5CC(F)C6)nc4c3F)c12] | |
| 127 | 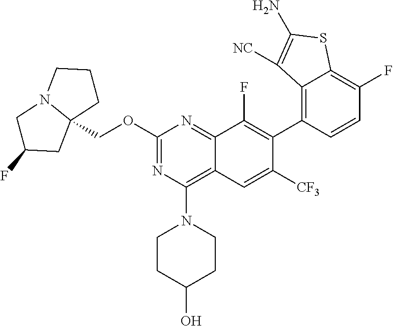 [SMILES: N#Cc1c(N)sc2c(F)ccc(-c3c(C(F)(F)F)cc4c(N5CCC(O)CC5)nc(OC[C@@]56CCCN5C[C@H](F)C6)nc4c3F)c12] | LCMS m/z [M + 1]: 663.0 1H NMR (400 MHz, CD3OD) δ 8.14 (s, 1H), 7.24- 7.21 (m, 1H), 7.04-7.00 (m, 1H), 5.51 (s, 0.5H), 5.38 (s, 0.6H), 4.56-4.42 (m, 2H), 4.33-4.30 (m, 2H), 4.06-4.00 (m, 1H), 3.74-3.48 (m, 5H), 3.29-3.22 (m, 1H), 2.59-2.03 (m, 8H), 1.78-1.70 (m, 2H). |
| 127a | 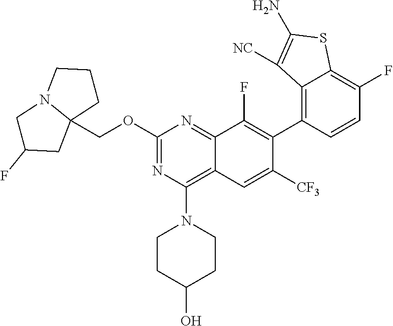 [SMILES: N#Cc1c(N)sc2c(F)ccc(-c3c(C(F)(F)F)cc4c(N5CCC(O)CC5)nc(OCC56CCCN5CC(F)C6)nc4c3F)c12] | |
| 128 | 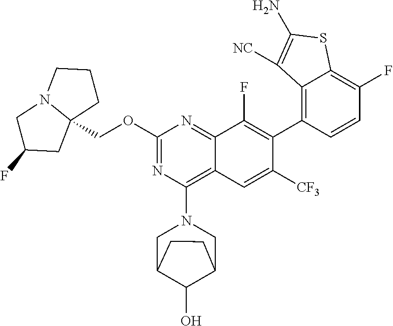 [SMILES: N#Cc1c(N)sc2c(F)ccc(-c3c(C(F)(F)F)cc4c(N5CC6CCC(C5)C6O)nc(OC[C@@]56CCCN5C[C@H](F)C6)nc4c3F)c12] | LCMS m/z [M + 1]: 689.0 1H NMR (400 MHz, CD3OD) δ 8.18 (s, 1H), 7.24- 7.17 (m, 1H), 7.04-6.95 (m, 1H), 5.39-5.34 (m, 0.5H), 5.25-5.21 (m, 0.5H), 4.48-4.39 (m, 1H), 4.38- 4.26 (m, 2H), 4.24-4.16 (m, 1H), 4.13-3.97 (m, 3H), 3.27-3.15 (m, 4H), 3.04-2.97 (m, 1H), 2.42-2.31 (m, 1H), 2.30-2.21 (m, 1H), 2.19-2.08 (m, 3H), 2.05-1.94 (m, 2H), 1.93-1.84 (m, 1H), 1.81-1.70 (m, 2H), 1.68- 1.57 (m, 2H). |
| 128a | 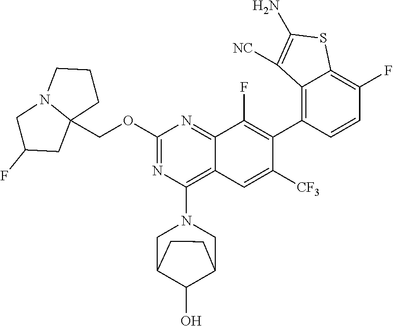 [SMILES: N#Cc1c(N)sc2c(F)ccc(-c3c(C(F)(F)F)cc4c(N5CC6CCC(C5)C6O)nc(OCC56CCCN5CC(F)C6)nc4c3F)c12] | |
| 129 | 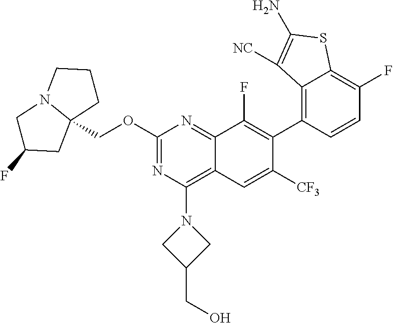 [SMILES: N#Cc1c(N)sc2c(F)ccc(-c3c(C(F)(F)F)cc4c(N5CC(CO)C5)nc(OC[C@@]56CCCN5C[C@H](F)C6)nc4c3F)c12] | LCMS m/z [M + 1]: 649.0 1H NMR (400 MHz, CD3OD) δ 8.09 (s, 1H), 7.17- 7.20 (m, 1H), 6.95-7.02 (m, 1H), 5.39 (s, 0.5H), 5.25 (s, 0.5H), 4.59 (s, 2H), 4.33-4.36 (m, 1H), 4.24-4.27 (m, 1H), 3.81-3.82 (m, 2H), 3.38-3.42 (m, 1H), 3.20- 3.27 (m, 1H), 3.02-3.10 (m, 2H), 2.81 (s, 1H), 1.88- 2.41 (m, 8H). |
| 129a | 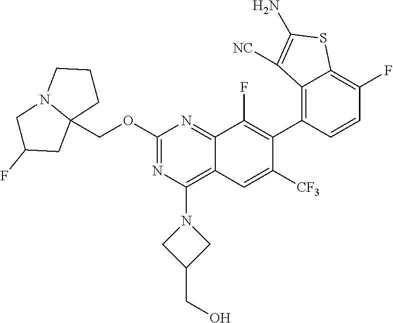 [SMILES: N#Cc1c(N)sc2c(F)ccc(-c3c(C(F)(F)F)cc4c(N5CC(CO)C5)nc(OCC56CCCN5CC(F)C6)nc4c3F)c12] | |
| 130 | 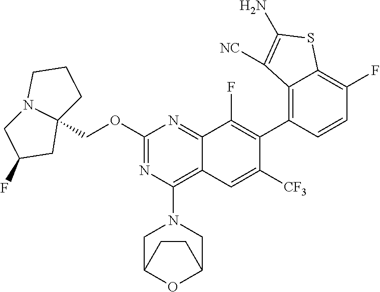 [SMILES: N#Cc1c(N)sc2c(F)ccc(-c3c(C(F)(F)F)cc4c(N5CC6CCC(C5)O6)nc(OC[C@@]56CCCN5C[C@H](F)C6)nc4c3F)c12] | LCMS m/z [M + 1]: 675.0 1H NMR (400 MHz, CD3OD) δ 8.12 (s, 1H), 7.17- 7.23 (m, 1H), 7.00 (t, 1H), 5.36 (s, 0.5H), 5.23 (s, 0.5H), 4.42-4.55 (m, 3H), 4.35-4.42 (m, 1H), 4.27- 4.32 (m, 1H), 4.18-4.23 (m, 1H), 3.70-3.83 (m, 2H), 3.15-3.25 (m, 3H), 2.97-3.05 (m, 1H), 2.30-2.40 (m, 1H), 2.10-2.30 (m, 3H), 1.82-2.02 (m, 6H). |
| 130a | 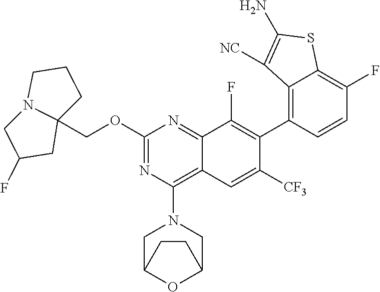 [SMILES: N#Cc1c(N)sc2c(F)ccc(-c3c(C(F)(F)F)cc4c(N5CC6CCC(C5)O6)nc(OCC56CCCN5CC(F)C6)nc4c3F)c12] | |
| 131 | 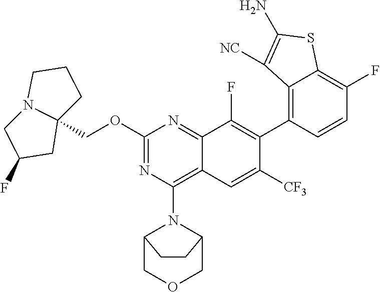 [SMILES: N#Cc1c(N)sc2c(F)ccc(-c3c(C(F)(F)F)cc4c(N5C6CCC5COC6)nc(OC[C@@]56CCCN5C[C@H](F)C6)nc4c3F)c12] | LCMS m/z [M + 1]: 675.1 1H NMR (400 MHz, DMCO) 8.08 (t, 3H), 7.23-7.26 (m, 1H), 7.13 (t, 1H), 5.33 (s, 0.5H), 5.20 (s, 0.5H), 4.80 (d, 2H), 4.10-4.14 (m, 1H), 4.02-4.04 (m. 1H), 3.93 (d. 1H), 3.86 (d, 1H), 3.76 (t., 2H), 3.00- 3.08 (m, 3H), 2.81-2.89 (m, 1H), 1.96-2.12 (m, 7H), 1.76-1.84 (m, 3H). |
| 131a | 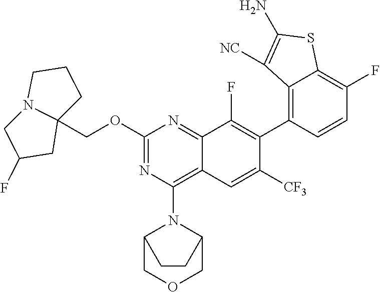 [SMILES: N#Cc1c(N)sc2c(F)ccc(-c3c(C(F)(F)F)cc4c(N5C6CCC5COC6)nc(OCC56CCCN5CC(F)C6)nc4c3F)c12] | |
| 132 | 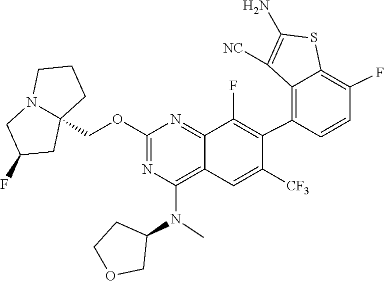 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@@H]1CCOC1] | LCMS m/z [M + 1]: 663.0 1H NMR (400 MHz, CD3OD) δ 8.30 (s, 1H), 7.17- 7.23 (m, 1H), 7.00 (t, 1H), 5.31-5.43 (s, 1.5H), 5.23 (s, 0.5H), 4.20-4.35 (m, 2H), 4.15-4.20 (m, 1H), 4.03-4.12 (m, 1H), 3.91-4.00 (m, 1H), 3.70- 3.80 (m, 1H), 3.40-3.55 (d, 3H), 3.17-3.27 (m, 3H), 2.97-3.05 (m, 1H), 2.48-2.60 (m, 1H), 2.10-2.40 (m, 4H), 1.83-2.05 (m, 3H). |
| 133 | 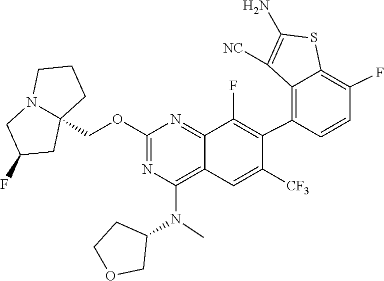 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@H]1CCOC1] | LCMS m/z [M + 1]: 663.1 1H NMR (400 MHz, CD3OD) δ 8.30 (s, 1H), 7.21 (t, 1H), 7.00 (t, 1H), 5.36 (s, 1.5H), 5.23 (s, 0.5H), 4.32 (d, 1H), 4.20-4.26 (m, 1H), 4.04-4.18 (m, 2H), 3.91- 3.99 (m, 1H), 3.71-3.78 (m, 1H), 3.39-3.49 (m, 3H), 3.19-3.25 (m, 2H), 2.98-3.05 (m, 1H), 2.50-2.58 (m, 1H), 2.17-2.38 (m, 5H), 1.86-2.02 (m, 3H). |
| 133a | 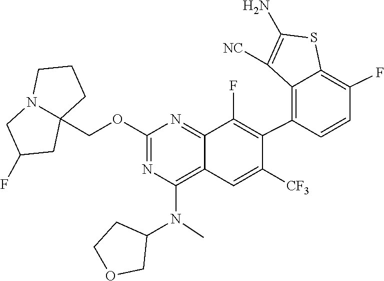 [SMILES: CN(c1nc(OCC23CCCN2CC(F)C3)nc2c(F)c(-c3ccc(F)c4sc(N)c(C#N)c34)c(C(F)(F)F)cc12)C1CCOC1] | |
| 134 | 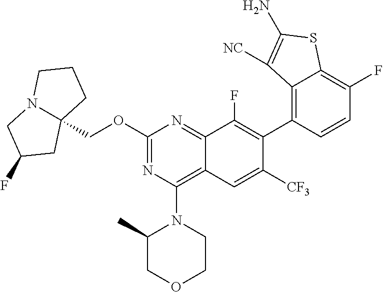 [SMILES: C[C@@H]1COCCN1c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 663 1H NMR (400 MHz, CD3OD) δ 8.06 (t, 3H), 7.24 (t, 1H), 7.11-7.16 (m, 1H), 5.34 (s, 0.5H), 5.20 (s, 0.5H), 4.60-4.69 (m, 1H), 4.02-4.14 (m, 3H), 3.91- 3.97 (m, 1H), 3.85 (t, 1H), 3.71-3.74 (m, 2H), 3.57- 3.63 (m, 1H), 3.00-3.09 (m, 3H), 2.81-2.85 (m, 1H), 2.00-2.14 (m, 3H), 1.76-1.84 (m, 3H), 1.45-1.49 (m, 3H). |
| 135 | 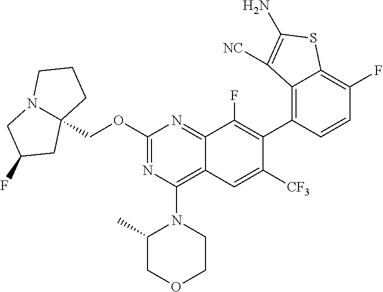 [SMILES: C[C@H]1COCCN1c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 663 1H NMR (400 MHz, DMSO_d6) δ 8.07 (t, 3H), 7.24 (t, 1H), 7.13 (t, 1H), 5.34 (s, 0.5H), 5.21 (s, 0.5H), 4.59-4.69 (m, 1H), 4.01-4.12 (m, 2H), 3.82-3.97 (m, 2H), 3.54-3.77 (m, 4H), 3.01-3.09 (m, 3H), 2.83 (s, 1H), 2.05 (t, 3H), 1.77-1.85 (m, 3H), 1.44-1.49 (m, 3H). |
| 135a | 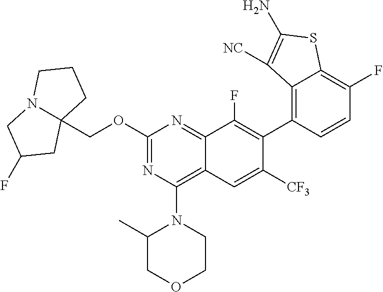 [SMILES: CC1COCCN1c1nc(OCC23CCCN2CC(F)C3)nc2c(F)c(-c3ccc(F)c4sc(N)c(C#N)c34)c(C(F)(F)F)cc12] | |
| 136 | 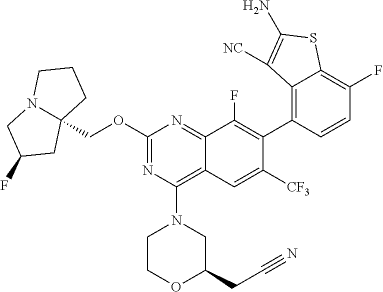 [SMILES: N#CC[C@@H]1CN(c2nc(OC[C@@]34CCCN3C[C@H](F)C4)nc3c(F)c(-c4ccc(F)c5sc(N)c(C#N)c45)c(C(F)(F)F)cc23)CCO1] | LCMS m/z [M + 1]: 688.2 1H NMR (400 MHz, CD3OD) δ 8.45 (s, 1H), 8.17 (s, 1H), 7.20 (t, 1H), 7.01 (t, 1H), 5.52 (s, 0.5H), 5.39 (s, 0.5H), 4.47-4.63 (m, 3H), 4.36-4.39 (m, 1H), 3.97-4.06 (m, 2H), 3.63-3.84 (m, 5H), 3.37-3.44 (m, 1H), 2.76-2.89 (m, 2H), 2.43-2.60 (m, 2H), 2.31-2.39 (m, 1H), 2.16-2.23 (m, 2H), 2.05-2.06 (m, 1H). |
| 137 | 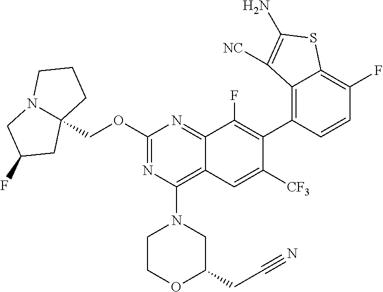 [SMILES: N#CC[C@H]1CN(c2nc(OC[C@@]34CCCN3C[C@H](F)C4)nc3c(F)c(-c4ccc(F)c5sc(N)c(C#N)c45)c(C(F)(F)F)cc23)CCO1] | LCMS m/z [M + 1]: 688.2 1H NMR (400 MHz, CD3OD) δ 8.14 (s, 1H), 7.19- 7.22 (m, 1H), 7.01 (t, 1H), 5.40 (s, 0.5H), 5.26 (s, 0.5H), 4.50-4.59 (m, 1H), 4.27-4.38 (m, 3H), 3.98- 4.06 (m, 2H), 3.79-3.83 (m, 1H), 3.67-3.70 (m, 1H), 3.32-3.42 (m, 3H), 3.03-3.13 (m, 1H), 2.75-2.87 (m, 2H), 2.16-2.43 (m, 3H), 1.99-2.08 (m, 2H), 1.90-1.96 (m, 1H). |
| 137a | 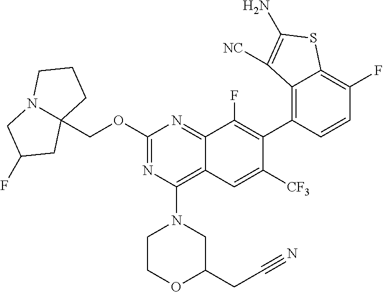 [SMILES: N#CCC1CN(c2nc(OCC34CCCN3CC(F)C4)nc3c(F)c(-c4ccc(F)c5sc(N)c(C#N)c45)c(C(F)(F)F)cc23)CCO1] | |
| 138 | 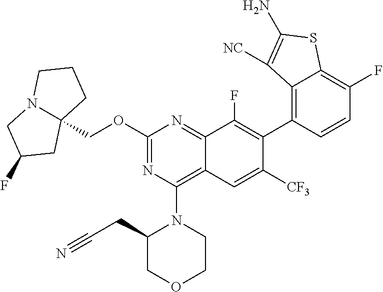 [SMILES: N#CC[C@@H]1COCCN1c1nc(OC[C@@]23CCCN2C[C@H](F)C3)nc2c(F)c(-c3ccc(F)c4sc(N)c(C#N)c34)c(C(F)(F)F)cc12] | |
| 139 | 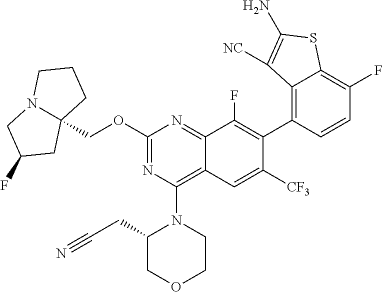 [SMILES: N#CC[C@H]1COCCN1c1nc(OC[C@@]23CCCN2C[C@H](F)C3)nc2c(F)c(-c3ccc(F)c4sc(N)c(C#N)c34)c(C(F)(F)F)cc12] | |
| 139a | 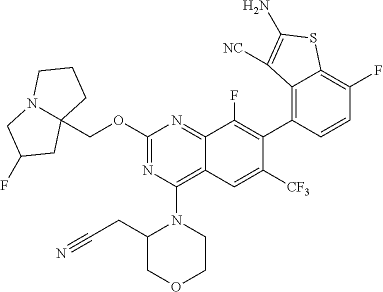 [SMILES: N#CCC1COCCN1c1nc(OCC23CCCN2CC(F)C3)nc2c(F)c(-c3ccc(F)c4sc(N)c(C#N)c34)c(C(F)(F)F)cc12] | |
| 140 | 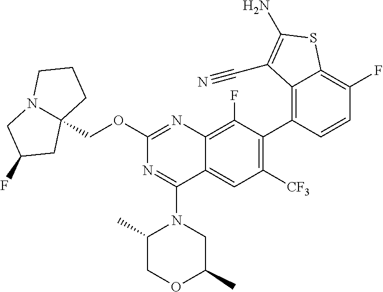 [SMILES: C[C@@H]1CN(c2nc(OC[C@@]34CCCN3C[C@H](F)C4)nc3c(F)c(-c4ccc(F)c5sc(N)c(C#N)c45)c(C(F)(F)F)cc23)[C@@H](C)CO1] | LCMS m/z [M + 1]: 677.1 1H NMR (400 MHz, CD3OD) δ 8.22 (s, 1H), 7.22- 7.24 (m, 1H), 7.00-7.05 (t, J = 9.0 Hz,1H), 5.51-5.63 (d, J = 51.4 Hz, 1H), 4.62-4.74 (m, 2H), 3.83-4.48 (m, 8H), 3.30-3.63 (m, 2H), 2.32-2.63 (m, 6H), 1.53- 1.54 (d, J = 6.4 Hz, 3H), 1.28-1.31 (m, 3H). |
| 140a | 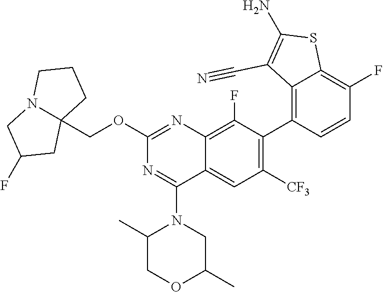 [SMILES: CC1CN(c2nc(OCC34CCCN3CC(F)C4)nc3c(F)c(-c4ccc(F)c5sc(N)c(C#N)c45)c(C(F)(F)F)cc23)C(C)CO1] | |
| 141 | 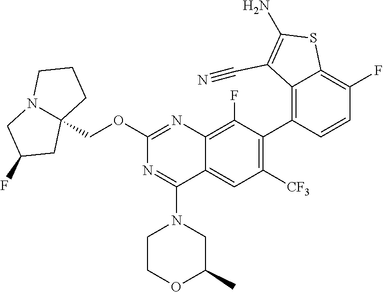 [SMILES: C[C@@H]1CN(c2nc(OC[C@@]34CCCN3C[C@H](F)C4)nc3c(F)c(-c4ccc(F)c5sc(N)c(C#N)c45)c(C(F)(F)F)cc23)CCO1] | LCMS m/z [M + 1]: 663.1 1H NMR (400 MHz, CD3OD) δ 8.18 (s, 1H), 7.21- 7.24 (m, 1H), 7.00-7.05 (t, J = 9.0 Hz, 1H), 5.49-5.62 (d, J = 51.6 Hz, 1H), 4.59-4.75 (m, 2H), 4.48-4.49 (m, 2H), 3.47-4.40 (m, 9H), 2.33-2.62 (m, 6H), 1.24- 1.56 (m, 3H). |
| 142 | 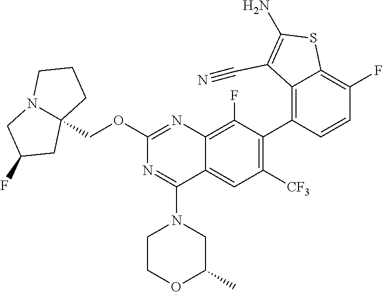 [SMILES: C[C@H]1CN(c2nc(OC[C@@]34CCCN3C[C@H](F)C4)nc3c(F)c(-c4ccc(F)c5sc(N)c(C#N)c45)c(C(F)(F)F)cc23)CCO1] | LCMS m/z [M + 1]: 663.2 1H NMR (400 MHz, CD3OD) δ 8.18 (s, 1H), 7.20- 7.24 (m, 1H), 7.02 (t, J = 9.2 Hz, 1H), 5.56 (d, J = 52.8 Hz, 1H), 4.62-4.75 (m, 2H), 4.42-4.50 (m, 2H), 3.71-4.09 (m, 6H), 3.57-3.69 (m, 1H), 3.40-3.50 (m, 1H), 3.20-3.27 (m, 1H), 2.51-2.78 (m, 2H), 2.33-2.42 (m, 3H), 2.10-2.15 (m, 1H), 1.25 (d, J = 5.6 Hz, 3H). |
| 142a | 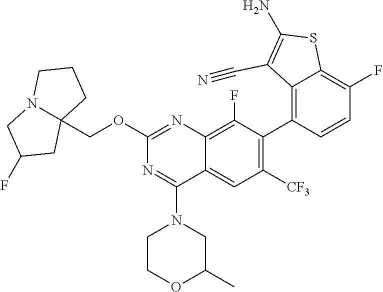 [SMILES: CC1CN(c2nc(OCC34CCCN3CC(F)C4)nc3c(F)c(-c4ccc(F)c5sc(N)c(C#N)c45)c(C(F)(F)F)cc23)CCO1] | |
| 143 | 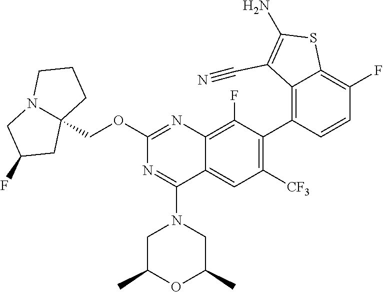 [SMILES: C[C@@H]1CN(c2nc(OC[C@@]34CCCN3C[C@H](F)C4)nc3c(F)c(-c4ccc(F)c5sc(N)c(C#N)c45)c(C(F)(F)F)cc23)C[C@H](C)O1] | LCMS m/z [M + 1]: 677.2 1H NMR (400 MHz, CD3OD) δ 8.17 (s, 1H), 7.20- 7.25 (m, 1H), 7.03 (t, J = 8.9 Hz, 1H), 5.55 (d, J = 51.9 Hz, 1H), 4.59-4.76 (m, 2H), 4.43-4.51 (m, 2H), 3.78-4.07 (m, 5H), 3.42-3.52 (s, 1H), 3.12-3.23 (m, 2H), 2.54-2.77 (m, 2H), 2.30-2.47 (m, 3H), 2.15 (s, 1H), 1.24 (d, J = 6 Hz, 6H). |
| 144 | 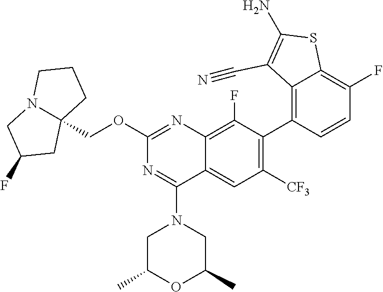 [SMILES: C[C@@H]1CN(c2nc(OC[C@@]34CCCN3C[C@H](F)C4)nc3c(F)c(-c4ccc(F)c5sc(N)c(C#N)c45)c(C(F)(F)F)cc23)C[C@@H](C)O1] | LCMS m/z [M + 1]: 677.2 1H NMR (400 MHz, CD3OD) δ 8.23 (s, 1H), 7.19- 7.27 (m, 1H), 6.99-7.07 (t, J = 9.0 Hz, 1H), 5.56 (d, J = 51.4 Hz, 1H), 4.59-4.76 (m, 2H), 4.21-4.31 (m, 2H), 3.79-4.12 (m, 7H), 3.42-3.51 (m, 1H), 2.55- 2.75 (m, 2H), 2.28-2.48 (m, 3H), 2.05-2.20 (m, 1H), 1.29 (d, J = 6 Hz, 6H). |
| 144a | 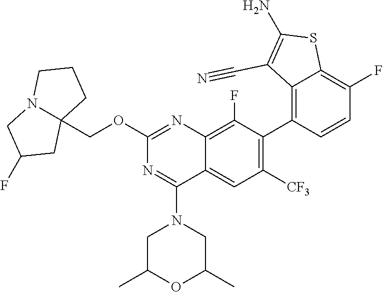 [SMILES: CC1CN(c2nc(OCC34CCCN3CC(F)C4)nc3c(F)c(-c4ccc(F)c5sc(N)c(C#N)c45)c(C(F)(F)F)cc23)CC(C)O1] | |
| 145 | 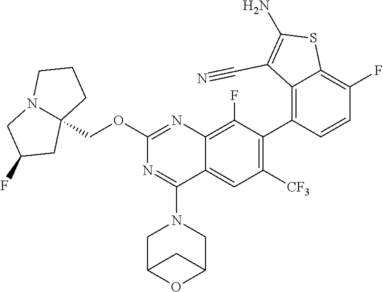 [SMILES: N#Cc1c(N)sc2c(F)ccc(-c3c(C(F)(F)F)cc4c(N5CC6CC(C5)O6)nc(OC[C@@]56CCCN5C[C@H](F)C6)nc4c3F)c12] | LCMS m/z [M + 1]: 661.0 1H NMR (400 MHz, CD3OD) δ 8.63 (s, 1H), 7.20- 7.24 (m, 1H), 7.02 (t, J = 9.2 Hz, 1H), 5.43 (d, J = 51.9 Hz, 1H), 4.43-4.59 (m, 5H), 4.30-4.38 (m, 2H), 3.47-3.78 (m, 3H), 1.95-2.56 (m, 8H), 1.29 (s, 2H). |
| 145a | 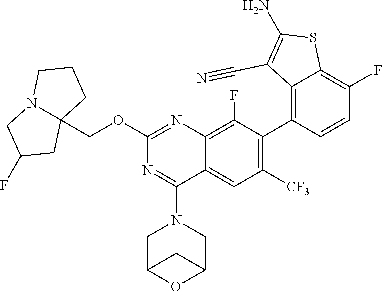 [SMILES: N#Cc1c(N)sc2c(F)ccc(-c3c(C(F)(F)F)cc4c(N5CC6CC(C5)O6)nc(OCC56CCCN5CC(F)C6)nc4c3F)c12] | |
| 146 | 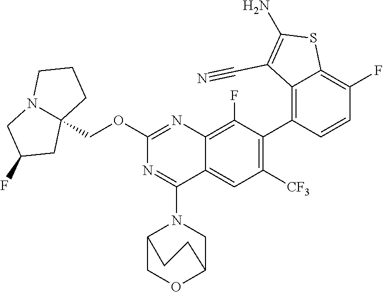 [SMILES: N#Cc1c(N)sc2c(F)ccc(-c3c(C(F)(F)F)cc4c(N5CC6CCC5CO6)nc(OC[C@@]56CCCN5C[C@H](F)C6)nc4c3F)c12] | LCMS m/z [M + 1]: 675.2 1H NMR (400 MHz, CD3OD) δ 8.41 (s, 1H), 7.19- 7.26 (m, 1H), 7.03 (t, J = 8.9 Hz, 1H), 5.55 (d, J = 50.5 Hz, 1H), 4.90-4.99 (m, 1H), 4.56-4.75 (m, 3H), 4.30-4.35 (m, 1H), 4.10-4.25 (m, 3H), 3.95-4.09 (m, 1H), 3.81-3.93 (m, 2H), 3.41-3.50 (m, 1H), 2.50- 2.75 (m, 2H), 2.05-2.46 (m, 7H), 1.82-1.93 (s, 1H). |
| 146a | 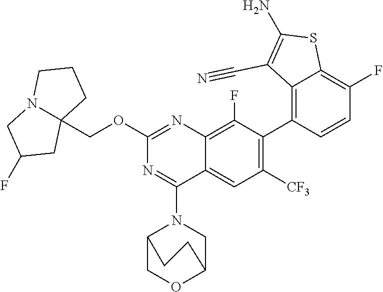 [SMILES: N#Cc1c(N)sc2c(F)ccc(-c3c(C(F)(F)F)cc4c(N5CC6CCC5CO6)nc(OCC56CCCN5CC(F)C6)nc4c3F)c12] | |
| 147 | 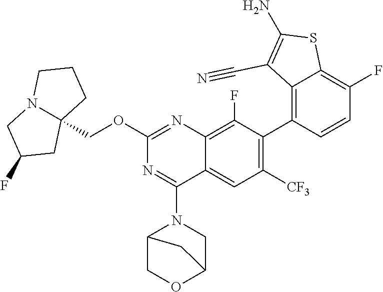 [SMILES: N#Cc1c(N)sc2c(F)ccc(-c3c(C(F)(F)F)cc4c(N5CC6CC5CO6)nc(OC[C@@]56CCCN5C[C@H](F)C6)nc4c3F)c12] | LCMS m/z [M + 1]: 661.0 1H NMR (400 MHz, CD3OD): δ 8.34 (s, 1H), 7.20- 7.25 (m, 1H), 7.02 (t, J = 8.8 Hz, 1H), 5.54 (d, J = 50.8 Hz, 1H), 4.60-4.84 (m, 3H), 4.30-4.34 (m, 1H), 3.85-4.07 (m, 6H), 3.40-3.55 (m, 1H), 2.52-2.62 (m, 2H), 2.33-2.46 (m, 3H), 2.08-2.18 (m, 4H). |
| 147a | 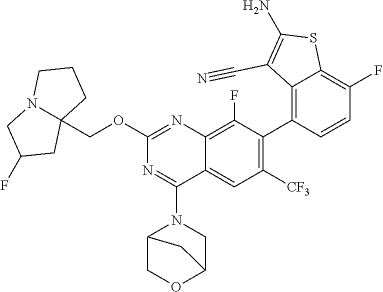 [SMILES: N#Cc1c(N)sc2c(F)ccc(-c3c(C(F)(F)F)cc4c(N5CC6CC5CO6)nc(OCC56CCCN5CC(F)C6)nc4c3F)c12] | |
| 148 | 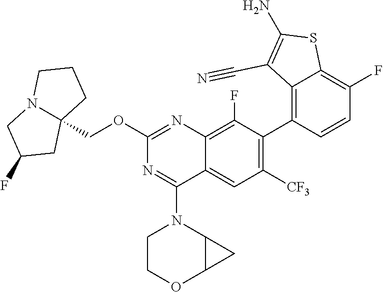 [SMILES: N#Cc1c(N)sc2c(F)ccc(-c3c(C(F)(F)F)cc4c(N5CCOC6CC65)nc(OC[C@@]56CCCN5C[C@H](F)C6)nc4c3F)c12] | LCMS m/z [M + 1]: 661.0 1HNMR (400 MHz, CD3OD) δ 8.15 (s, 1H), 7.15- 7.26 (m, 1H), 6.95-7.05 (m, 1H), 5.37 (s, 0.5H), 5.24 (s, 0.5H), 4.10-4.36 (m, 3H), 4.00-4.08 (m, 1H), 3.87-3.98 (m, 2H), 3.54-3.65 (m, 1H), 3.33-3.42 (m, 1H), 3.15-3.28 (m, 3H), 2.97-3.07 (m, 1H), 2.10-2.42 (m, 3H), 1.83-2.06 (m, 3H), 1.12-1.25 (m, 2H). |
| 148a | 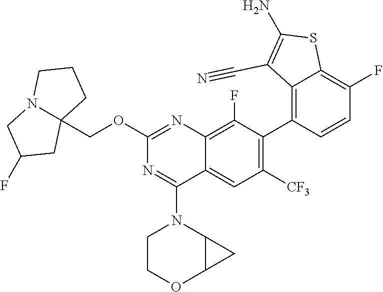 [SMILES: N#Cc1c(N)sc2c(F)ccc(-c3c(C(F)(F)F)cc4c(N5CCOC6CC65)nc(OCC56CCCN5CC(F)C6)nc4c3F)c12] | |
| 149 | 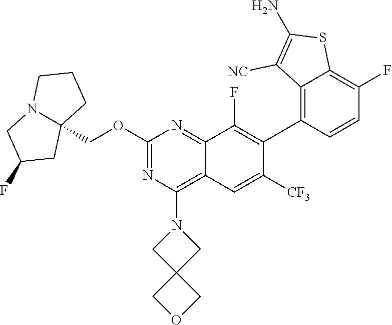 [SMILES: N#Cc1c(N)sc2c(F)ccc(-c3c(C(F)(F)F)cc4c(N5CC6(COC6)C5)nc(OC[C@@]56CCCN5C[C@H](F)C6)nc4c3F)c12] | LCMS m/z [M + 1]: 661.17 1H NMR (400 MHz, CD3OD) δ 8.07 (s, 1H), 7.22- 7.15 (m, 1H), 7.04-6.97 (m, 1H), 5.44 (s, 0.5H), 5.31 (s, 0.5H), 4.91 (s, 7H), 4.47-4.40 (m, 1H), 4.38-4.32 (m, 1H), 3.60-3.37 (m, 3H), 3.19-3.11 (m,1H), 2.50- 1.86 (m, 7H). |
| 149a | 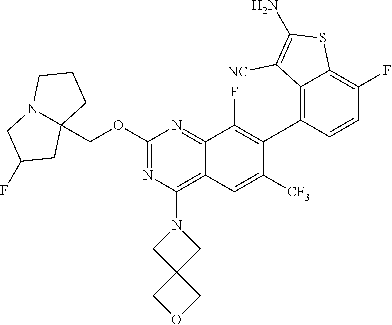 [SMILES: N#Cc1c(N)sc2c(F)ccc(-c3c(C(F)(F)F)cc4c(N5CC6(COC6)C5)nc(OCC56CCCN5CC(F)C6)nc4c3F)c12] | |
| 150 | 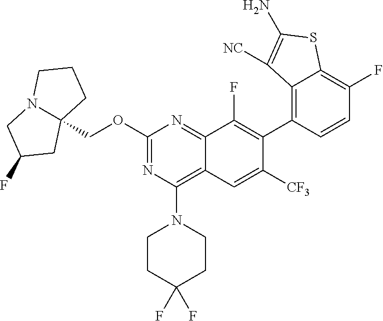 [SMILES: N#Cc1c(N)sc2c(F)ccc(-c3c(C(F)(F)F)cc4c(N5CCC(F)(F)CC5)nc(OC[C@@]56CCCN5C[C@H](F)C6)nc4c3F)c12] | LCMS m/z [M + 1]: 683.0 1H NMR (400 MHz, CD3OD) δ 8.13 (d, 1H), 7.19- 7.23 (m, 1H), 6.98-7.03 (m, 1H), 5.37 (m, 0.5H), 5.23 (m, 0.5H), 4.31-4.34 (m, 1H), 4.23-4.27 (m, 1H), 4.04-4.05 (m, 4H), 3.19-3.25 (m, 3H), 2.98-3.02 (m, 1H), 2.12-2.34 (m, 7H), 1.95-2.03 (m, 2H), 1.87- 1.93 (m, 1H). |
| 150a | 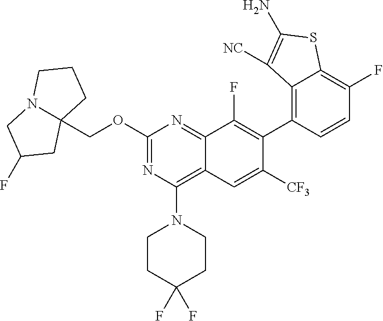 [SMILES: N#Cc1c(N)sc2c(F)ccc(-c3c(C(F)(F)F)cc4c(N5CCC(F)(F)CC5)nc(OCC56CCCN5CC(F)C6)nc4c3F)c12] | |
| 151 | 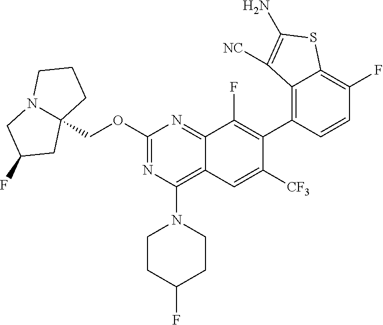 [SMILES: N#Cc1c(N)sc2c(F)ccc(-c3c(C(F)(F)F)cc4c(N5CCC(F)CC5)nc(OC[C@@]56CCCN5C[C@H](F)C6)nc4c3F)c12] | LCMS m/z [M + 1]: 665.1 1H NMR (400 MHz, CD3OD) δ 8.10 (s, 1H), 7.09- 7.13 (m, 1H), 6.90 (t, 1H), 5.27 (s, 0.5H), 5.14 (s, 0.5H), 4.95 (s, 0.5H), 4.52 (s, 0.5H), 4.21-4.24 (m, 1H), 4.13-4.16 (m, 1H), 3.87-3.95 (m, 4H), 3.10-3.17 (m, 3H), 2.69-2.95 (m, 1H), 2.00-2.26 (m, 5H), 1.78- 1.96 (m, 5H). |
| 151a | 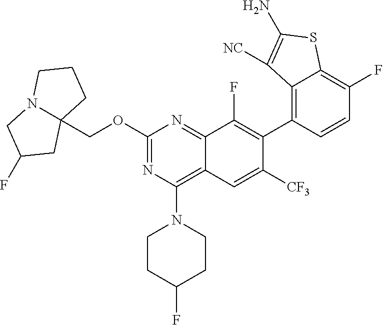 [SMILES: N#Cc1c(N)sc2c(F)ccc(-c3c(C(F)(F)F)cc4c(N5CCC(F)CC5)nc(OCC56CCCN5CC(F)C6)nc4c3F)c12] | |
| 152 | 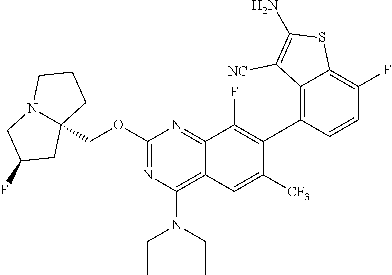 [SMILES: CCN(CC)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 635.0 1H NMR (400 MHz, CD3OD) δ 8.21 (s, 1H), 7.25- 7.16 (m, 1H), 7.05-6.95 (m, 1H), 5.44-5.37 (m, 0.5H), 5.30-5.24 (m, 0.5H), 4.43-4.24 (m, 2H), 3.98- 3.75 (m, 4H), 3.49-3.32 (m, 2H), 3.13-3.04 (m, 1H), 2.46-1.83 (m, 7H), 1.47 (t, J = 7.2 Hz, 6H). |
| 152a | 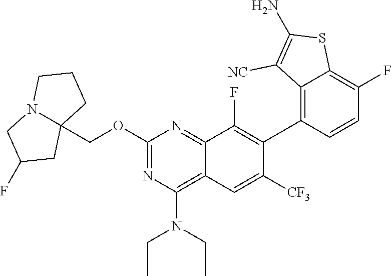 [SMILES: CCN(CC)c1nc(OCC23CCCN2CC(F)C3)nc2c(F)c(-c3ccc(F)c4sc(N)c(C#N)c34)c(C(F)(F)F)cc12] | |
| 153 | 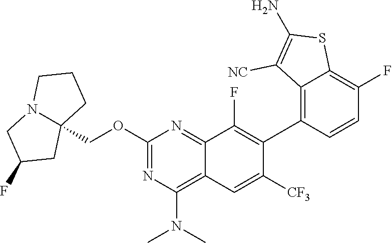 [SMILES: CN(C)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 607.0 1H NMR (400 MHz, CD3OD) δ 8.352 (s, 1H), 7.17- 7.23 (m, 1H), 7.04-6.98 (m, 1H), 5.385 (s, 0.5H), 5.252 (s, 0.5H), 4.38-4.32 (m, 1H), 4.29-4.23 (m, 1H), 3.57-3.446 (m, 6H), 3.29-3.20 (m, 3H), 3.07- 2.99 (m, 1H), 2.35-2.13 (m, 3H), 2.05-1.88 (m, 3H). |
| 153a | 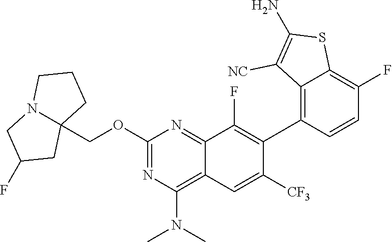 [SMILES: CN(C)c1nc(OCC23CCCN2CC(F)C3)nc2c(F)c(-c3ccc(F)c4sc(N)c(C#N)c34)c(C(F)(F)F)cc12] | |
| 154 | 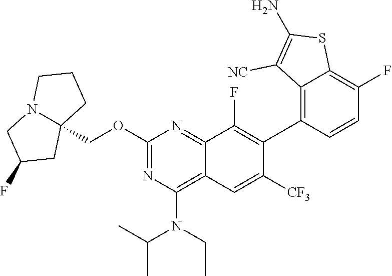 [SMILES: CCN(c1nc(OC[C@@]23CCCN2C[C@H](F)C3)nc2c(F)c(-c3ccc(F)c4sc(N)c(C#N)c34)c(C(F)(F)F)cc12)C(C)C] | LCMS m/z [M + 1]: 649 1H NMR (400 MHz, CD3OD) δ 8.18 (s, 1H), 7.17- 7.25 (m, 1H), 7.00 (t, 1H), 5.37 (s, 0.5H), 5.23 (s, 0.5H), 4.80-4.86 (m, 2H), 4.18-4.37 (m, 2H), 3.71- 3.85 (m, 2H), 3.21-3.25 (m, 1H), 2.98-3.07 (m, 1H), 1.83-2.40 (m, 7H), 1.33-1.48 (m, 9H). |
| 154a | 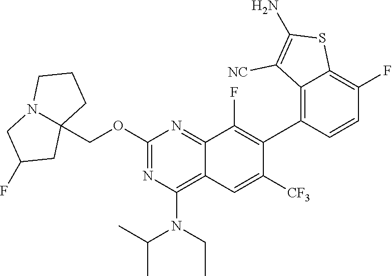 [SMILES: CCN(c1nc(OCC23CCCN2CC(F)C3)nc2c(F)c(-c3ccc(F)c4sc(N)c(C#N)c34)c(C(F)(F)F)cc12)C(C)C] | |
| 155 | 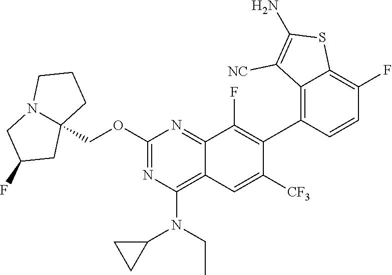 [SMILES: CCN(c1nc(OC[C@@]23CCCN2C[C@H](F)C3)nc2c(F)c(-c3ccc(F)c4sc(N)c(C#N)c34)c(C(F)(F)F)cc12)C1CC1] | LCMS m/z [M + 1]: 646.9 1H NMR (400 MHz, CD3OD) δ 8.84 (s, 1H), 7.20- 7.23 (m, 1H), 7.00 (t, 1H), 5.37 (s, 0.5H), 5.23 (s, 0.5H), 4.29 (d, 1H), 4.21 (d, 1H), 4.05-4.12 (m, 1H), 3.92-3.98 (m, 1H), 3.50-3.54 (m, 1H), 3.21-3.26 (m, 3H), 2.99-3.05 (m, 1H), 2.21-2.37 (m, 2H), 2.11-2.16 (m, 1H), 1.86-2.01 (m, 3H), 1.43 (t, 3H), 1.04-1.10 (m, 2H), 0.81-0.85 (m, 2H). |
| 155a | 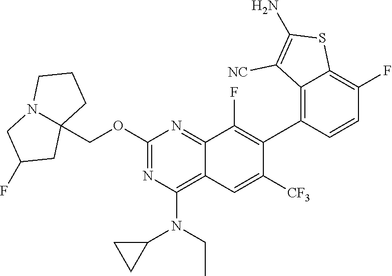 [SMILES: CCN(c1nc(OCC23CCCN2CC(F)C3)nc2c(F)c(-c3ccc(F)c4sc(N)c(C#N)c34)c(C(F)(F)F)cc12)C1CC1] | |
| 156 | 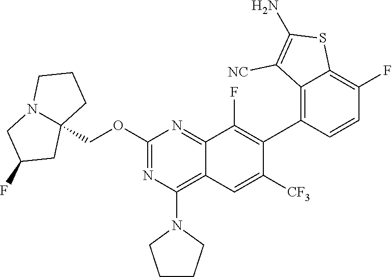 [SMILES: N#Cc1c(N)sc2c(F)ccc(-c3c(C(F)(F)F)cc4c(N5CCCC5)nc(OC[C@@]56CCCN5C[C@H](F)C6)nc4c3F)c12] | LCMS m/z [M + 1]: 633.0 1H NMR (400 MHz, CD3OD) δ 8.38 (s, 1H), 7.12- 7.10 (m, 1H), 6.94-6.90 (m, 1H), 5.42 (s, 0.55H), 5.29 (s, 0.55H), 4.65 (s, 1H), 4.50-4.35 (m, 2H), 3.95 (s, 4H), 3.66-3.38 (m, 3H), 3.03 (s, 1H), 2.59-1.94 (m, 10H). |
| 156a | 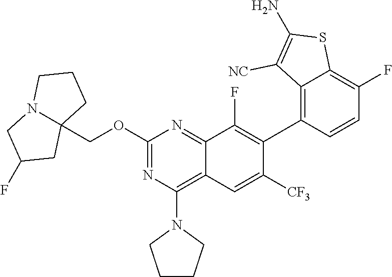 [SMILES: N#Cc1c(N)sc2c(F)ccc(-c3c(C(F)(F)F)cc4c(N5CCCC5)nc(OCC56CCCN5CC(F)C6)nc4c3F)c12] | |
| 157 | 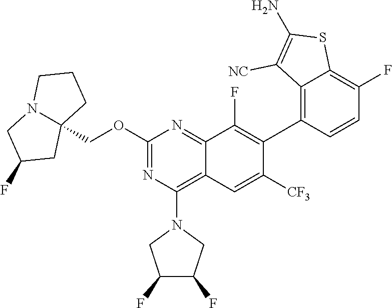 [SMILES: N#Cc1c(N)sc2c(F)ccc(-c3c(C(F)(F)F)cc4c(N5C[C@@H](F)[C@@H](F)C5)nc(OC[C@@]56CCCN5C[C@H](F)C6)nc4c3F)c12] | LCMS m/z [M + 1]: 669.2 1H NMR (400 MHz, CD3OD) δ 8.37 (s, 1H), 7.26- 7.18 (m, 1H), 7.06-6.97 (m, 1H), 5.62-5.57 (m, 0.5H), 5.52-5.49 (m, 0.5H), 5.48-5.43 (m, 1H), 5.39- 5.32 (m, 1H), 4.72-4.56 (m, 2H), 4.53-4.20 (m, 4H), 4.02-3.66 (m, 3H), 3.49-3.34 (m, 1H), 2.70-2.07 (m, 6H). |
| 158 | 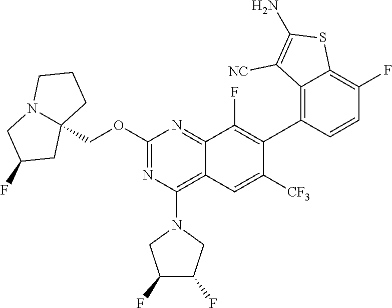 [SMILES: N#Cc1c(N)sc2c(F)ccc(-c3c(C(F)(F)F)cc4c(N5C[C@H](F)[C@@H](F)C5)nc(OC[C@@]56CCCN5C[C@H](F)C6)nc4c3F)c12] | LCMS m/z [M + 1]: 669.0 1H NMR (400 MHz, CD3OD) δ 8.45 (s, 1H), 7.24- 7.20 (m, 1H), 7.04-6.99 (m, 1H), 5.54-5.36 (m, 3H), 4.57-4.39 (m, 5H), 3.66-3.48 (m, 3H), 3.24-3.13 (m, 2H), 2.52-2.01 (m, 6H). |
| 158a | 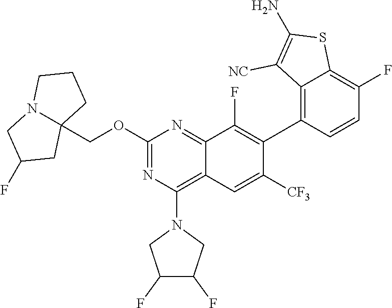 [SMILES: N#Cc1c(N)sc2c(F)ccc(-c3c(C(F)(F)F)cc4c(N5CC(F)C(F)C5)nc(OCC56CCCN5CC(F)C6)nc4c3F)c12] | |
| 159 | 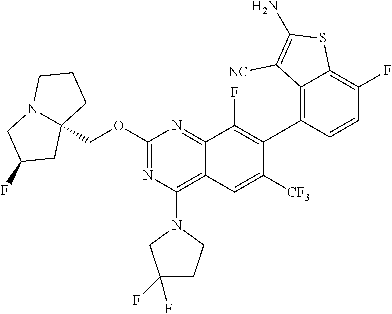 [SMILES: N#Cc1c(N)sc2c(F)ccc(-c3c(C(F)(F)F)cc4c(N5CCC(F)(F)C5)nc(OC[C@@]56CCCN5C[C@H](F)C6)nc4c3F)c12] | LCMS m/z [M + 1]: 669.16 1H NMR (400 MHz, CD3OD) δ 8.35 (s, 1H), 7.23- 7.17 (m, 1H), 7.04-6.97 (m, 1H), 5.37 (m, 0.5H), 5.23 (m, 0.5H), 4.46-4.18 (m, 6H), 3.27-3.07 (m, 3H), 3.04-2.98 (m, 1H), 2.69-2.57 (m, 2H), 2.39-1.83 (m, 7H). |
| 159a | 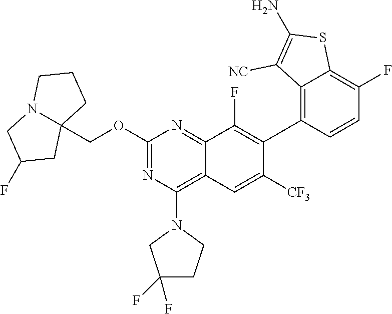 [SMILES: N#Cc1c(N)sc2c(F)ccc(-c3c(C(F)(F)F)cc4c(N5CCC(F)(F)C5)nc(OCC56CCCN5CC(F)C6)nc4c3F)c12] | |
| 160 | 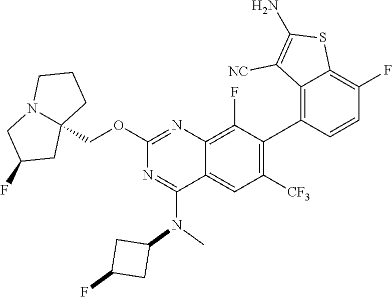 | |
| 161 | 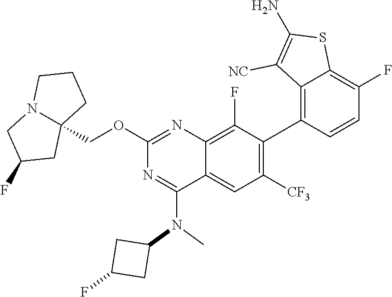 | LCMS m/z [M + 1]: 655.0 1H NMR (400 MHz, CD3OD) δ 8.269 (s, 1H), 7.26- 7.20 (m, 1H), 7.028 (t, 1H), 5.620 (s, 0.5H), 5.49 (s, 0.5H), 5.317 (s, 0.5H), 5.174 (s, 0.5H), 5.16-5.11 (m, 1H), 4.76-4.59 (m, 3H), 4.09-3.83 (m, 3H), 3.46 (s, 3H), 2.85-2.55 (m, 6H), 2.52-2.16 (m, 4H). |
| 161a | 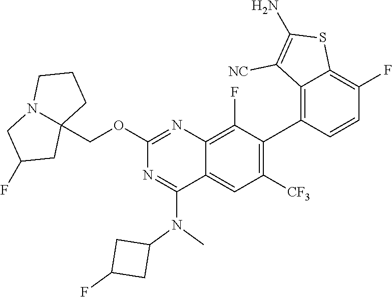 [SMILES: CN(c1nc(OCC23CCCN2CC(F)C3)nc2c(F)c(-c3ccc(F)c4sc(N)c(C#N)c34)c(C(F)(F)F)cc12)C1CC(F)C1] | |
| 162 | 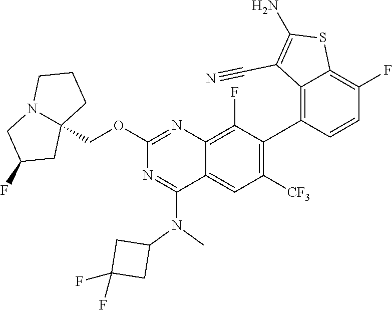 [SMILES: CN(c1nc(OC[C@@]23CCCN2C[C@H](F)C3)nc2c(F)c(-c3ccc(F)c4sc(N)c(C#N)c34)c(C(F)(F)F)cc12)C1CC(F)(F)C1] | LCMS m/z [M + 1]: 683.0 1H NMR (400 MHz, CD3OD) δ 8.24 (s, 1H), 7.21 (t, 1H), 7.01 (t, 1H), 5.38 (s, 0.5H), 5.23 (s, 0.5H), 4.52- 4.50 (m, 1H), 4.31-4.20 (m, 2H), 3.47 (s, 3H), 3.25- 2.92 (m, 8H), 2.36-2.12 (m, 3H), 2.03-1.87 (m, 3H). |
| 162a | 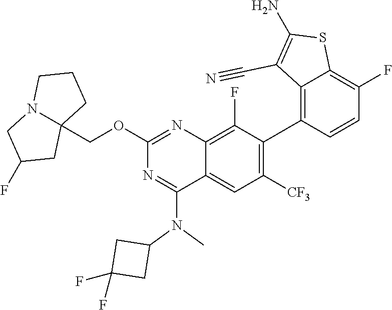 [SMILES: CN(c1nc(OCC23CCCN2CC(F)C3)nc2c(F)c(-c3ccc(F)c4sc(N)c(C#N)c34)c(C(F)(F)F)cc12)C1CC(F)(F)C1] | |
| 163 | 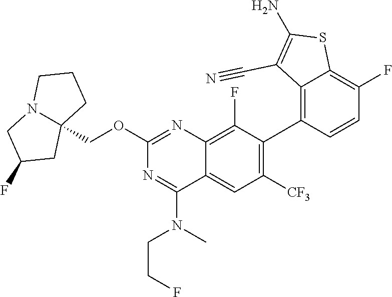 [SMILES: CN(CCF)c1nc(OC[C@@]23CCCN2C[C@H](F)C3)nc2c(F)c(-c3ccc(F)c4sc(N)c(C#N)c34)c(C(F)(F)F)cc12] | |
| 163a | 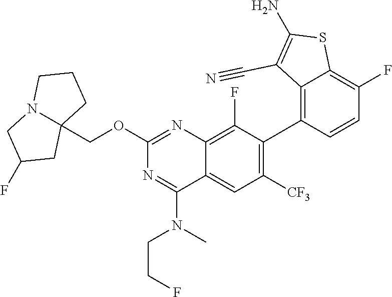 [SMILES: CN(CCF)c1nc(OCC23CCCN2CC(F)C3)nc2c(F)c(-c3ccc(F)c4sc(N)c(C#N)c34)c(C(F)(F)F)cc12] | |
| 164 | 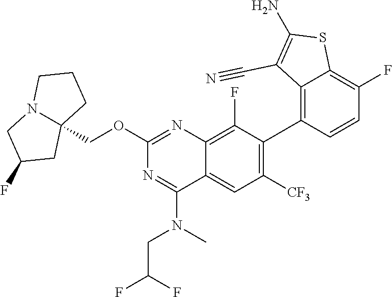 [SMILES: CN(CC(F)F)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 657.2 1H NMR (400 MHz, CD3OD) δ 8.43 (s, 1H), 7.19- 7.23 (m, 1H), 6.98-7.03 (m, 1H), 6.51-6.53 (m, 0.3H), 6.37-6.39 (m, 0.5H), 6.23-6.25 (m, 0.3H), 5.37 (s, 0.5H), 5.24 (s, 0.5H), 4.22-4.32 (m, 4H), 3.66-3.72 (m, 3H), 3.18-3.26 (m, 3H), 2.99-3.05 (m, 1H), 2.11-2.39 (m, 3H), 1.88-2.02 (m, 3H). |
| 164a | 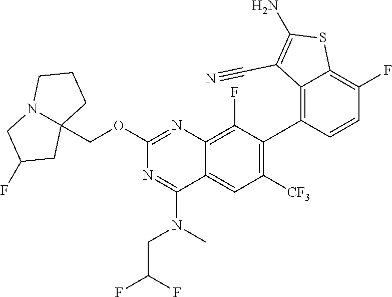 [SMILES: CN(CC(F)F)c1nc(OCC23CCCN2CC(F)C3)nc2c(F)c(-c3ccc(F)c4sc(N)c(C#N)c34)c(C(F)(F)F)cc12] | |
| 165 | 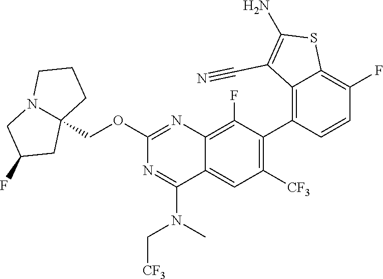 [SMILES: CN(CC(F)(F)F)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 674.7 1H NMR (400 MHz, CD3OD) δ 8.486 (s, 1H), 7.26- 7.21 (m, 1H), 7.031 (t, 1H), 5.630 (s, 0.5H), 5.500 (s, 0.5H), 4.87-4.82 (m, 2H), 4.72-4.600 (m, 2H), 4.037- 3.862 (m, 3H), 3.766 (s, 3H), 3.49-3.43 (m, 1H), 2.80-2.46 (m, 3H), 2.44-2.30 (m, 3H). |
| 165a | 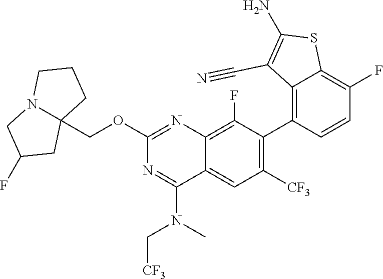 [SMILES: CN(CC(F)(F)F)c1nc(OCC23CCCN2CC(F)C3)nc2c(F)c(-c3ccc(F)c4sc(N)c(C#N)c34)c(C(F)(F)F)cc12] | |
| 166 | 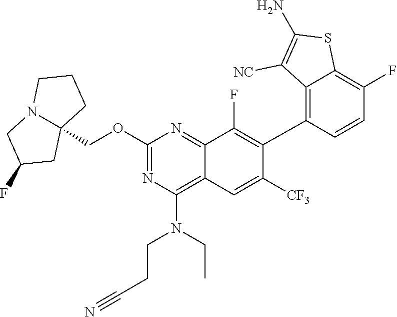 [SMILES: CCN(CCC#N)c1nc(OC[C@@]23CCCN2C[C@H](F)C3)nc2c(F)c(-c3ccc(F)c4sc(N)c(C#N)c34)c(C(F)(F)F)cc12] | |
| 166a | 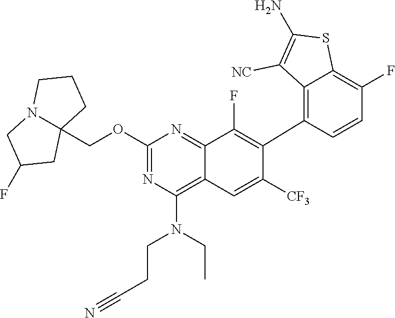 [SMILES: CCN(CCC#N)c1nc(OCC23CCCN2CC(F)C3)nc2c(F)c(-c3ccc(F)c4sc(N)c(C#N)c34)c(C(F)(F)F)cc12] | |
| 167 | 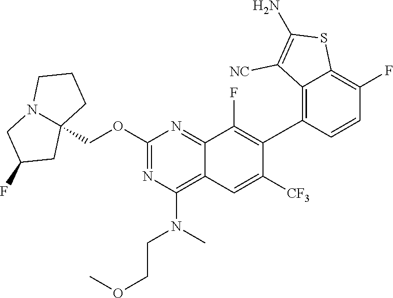 [SMILES: COCCN(C)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 651.0 1HNMR (400 MHz, CD3OD) δ 8.69 (s, 1H), 7.17- 7.25 (m, 1H), 7.02 (t, 1H), 5.61 (s, 0.5H), 5.49 (s, 0.5H), 4.57-4.75 (m, 2H), 4.05-4.15 (m, 2H), 3.77- 4.05 (m, 5H), 3.58 (s, 3H), 3.37-3.51 (m, 4H), 2.55- 2.75 (m, 2H), 2.25-2.45 (m, 3H), 2.08-2.20 (m, 1H). |
| 167a | 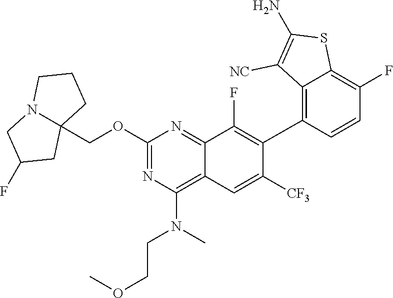 [SMILES: COCCN(C)c1nc(OCC23CCCN2CC(F)C3)nc2c(F)c(-c3ccc(F)c4sc(N)c(C#N)c34)c(C(F)(F)F)cc12] | |
| 168 | 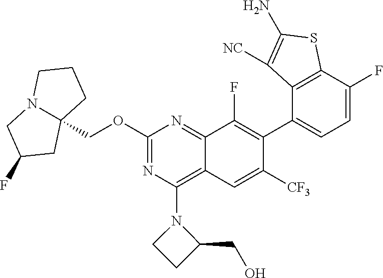 [SMILES: N#Cc1c(N)sc2c(F)ccc(-c3c(C(F)(F)F)cc4c(N5CC[C@@H]5CO)nc(OC[C@@]56CCCN5C[C@H](F)C6)nc4c3F)c12] | LCMS m/z [M + 1]: 649.0 1H NMR (400 MHz, CD3OD) δ 8.089 (d, 1H), 7.15- 7.23 (m, 1H), 6.96-7.04 (m, 1H), 5.368 (s, 0.5H), 5.231 (s, 0.5H), 4.975 (s, 1H), 4.77-4.82 (m, 1H), 4.63-4.72 (m, 1H), 4.17-4.25 (m, 2H), 3.802-3.875 (m, 1H), 3.183-3.275 (m, 3H), 2.987-3.053 (m, 1H), 2.605-2.67 (m, 1H), 2.49-2.56 (m, 1H), 2.29-2.395 (m, 1H), 2.07-2.29 (m, 3H), 1.855-2.045 (m, 3H). |
| 169 | 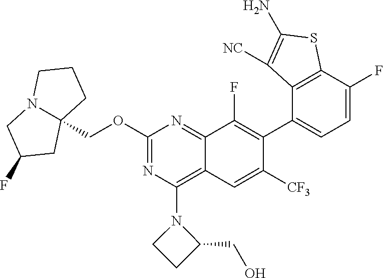 [SMILES: N#Cc1c(N)sc2c(F)ccc(-c3c(C(F)(F)F)cc4c(N5CC[C@H]5CO)nc(OC[C@@]56CCCN5C[C@H](F)C6)nc4c3F)c12] | LCMS m/z [M + 1]: 649.0 1H NMR (400 MHz, CD3OD) δ 8.09 (s, 1H), 7.15- 7.23 (m, 1H), 7.00 (t, 1H), 5.36 (s, 0.5H), 5.23 (s, 0.5H), 4.97 (s, 1H), 4.62-4.82 (m, 2H), 4.27-4.35 (m, 1H), 4.12-4.25 (m, 2H), 3.80-3.86 (m, 1H), 3.19-3.27 (m, 2H), 2.97-3.05 (m, 1H), 2.60-2.68 (m, 1H), 2.50- 2.57 (m, 1H), 2.30-2.38 (m, 1H), 2.07-2.27 (m, 3H), 1.82-2.03 (m, 3H). |
| 169a | 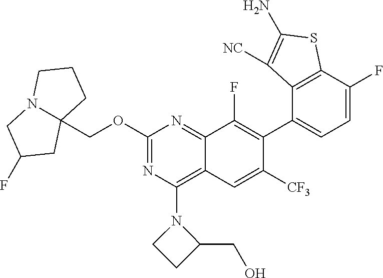 [SMILES: N#Cc1c(N)sc2c(F)ccc(-c3c(C(F)(F)F)cc4c(N5CCC5CO)nc(OCC56CCCN5CC(F)C6)nc4c3F)c12] | |
| 170 | 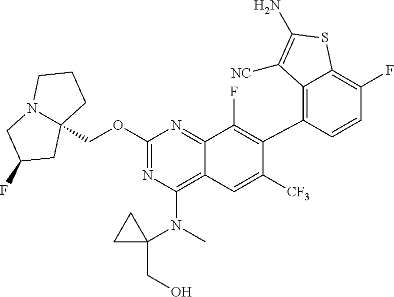 [SMILES: CN(c1nc(OC[C@@]23CCCN2C[C@H](F)C3)nc2c(F)c(-c3ccc(F)c4sc(N)c(C#N)c34)c(C(F)(F)F)cc12)C1(CO)CC1] | LCMS m/z [M + 1]: 663.1. 1H NMR (400 MHz, CD3OD) δ 9.05 (d, 1H), 7.22- 7.19 (m, 1H), 7.03-6.98 (m, 1H), 5.38 (s, 0.5H), 5.24 (s, 0.5H), 4.35-4.24 (m, 2H), 4.07 (d, 1H), 3.92 (d, 1H), 3.50 (s, 3H), 3.26 (s, 1H), 3.07-3.01 (m, 1H), 2.36-2.13 (m, 4H), 2.04-1.88 (m, 4H), 1.01-1.02 (m, 4H). |
| 170a | 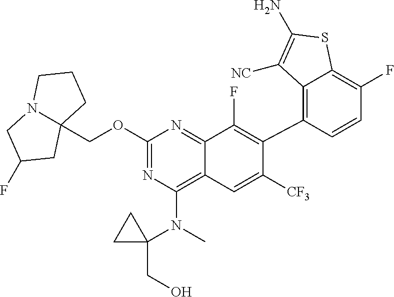 [SMILES: CN(c1nc(OCC23CCCN2CC(F)C3)nc2c(F)c(-c3ccc(F)c4sc(N)c(C#N)c34)c(C(F)(F)F)cc12)C1(CO)CC1] | |
| 171 | 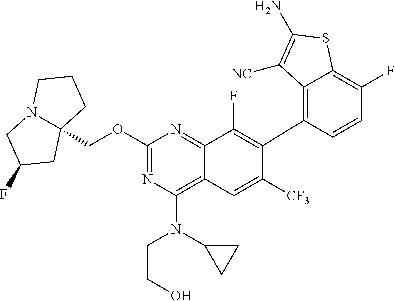 [SMILES: N#Cc1c(N)sc2c(F)ccc(-c3c(C(F)(F)F)cc4c(N(CCO)C5CC5)nc(OC[C@@]56CCCN5C[C@H](F)C6)nc4c3F)c12] | LCMS m/z [M + 1]: 663.0 1H NMR (400 MHz, CD3OD) δ 8.90 (s, 1H), 7.20- 7.23 (m, 1H), 7.00 (t, 1H), 5.38 (s, 0.5H), 5.22 (s, 0.5H), 4.17-4.28 (m, 3H), 3.95-4.04 (m, 3H), 3.56- 3.59 (m, 1H), 3.19-3.26 (m, 3H), 3.00-3.01 (m, 1H), 2.10-2.32 (m, 3H), 1.85-1.99 (m, 3H), 1.07-1.10 (m, 2H), 0.80-0.83 (m, 2H). |
| 171a | 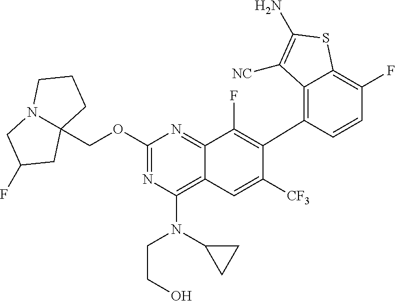 [SMILES: N#Cc1c(N)sc2c(F)ccc(-c3c(C(F)(F)F)cc4c(N(CCO)C5CC5)nc(OCC56CCCN5CC(F)C6)nc4c3F)c12] | |
| 174 | 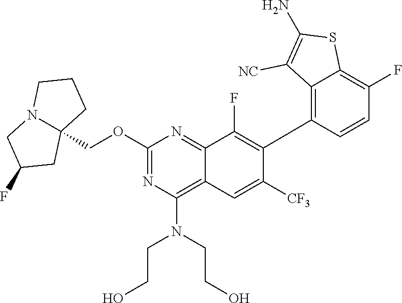 [SMILES: N#Cc1c(N)sc2c(F)ccc(-c3c(C(F)(F)F)cc4c(N(CCO)CCO)nc(OC[C@@]56CCCN5C[C@H](F)C6)nc4c3F)c12] | LCMS m/z [M + 1]: 667.0 1H NMR (400 MHz, CD3OD) δ 8.81 (s, 1H), 7.17- 7.24 (m, 1H), 6.97-7.06 (m, 1H), 5.40 (s, 0.5H), 5.26 (s, 0.5H), 4.25-4.41 (m, 2H), 3.98-4.10 (m, 6H), 3.66-3.82 (m, 1H), 3.33-3.41 (m, 1H), 2.95-3.15 (m, 2H), 1.81-2.48 (m, 8H). |
| 174a | 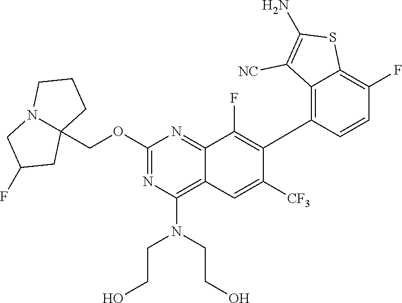 [SMILES: N#Cc1c(N)sc2c(F)ccc(-c3c(C(F)(F)F)cc4c(N(CCO)CCO)nc(OCC56CCCN5CC(F)C6)nc4c3F)c12] | |
| 175 | 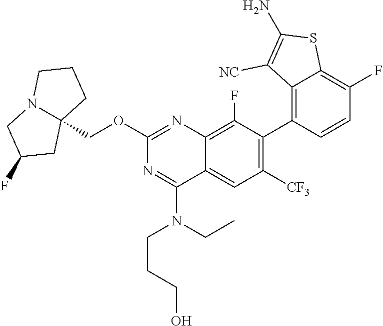 [SMILES: CCN(CCCO)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 665.3 1H NMR (400 MHz, CD3OD) δ 8.23 (s, 1H), 7.22- 7.19 (m, 1H), 7.00 (t, 1H), 5.38 (s, 0.5H), 5.25 (s, 0.5H), 4.35-4.24 (m, 2H), 3.96-3.863 (m, 4H), 3.71 (t, 2H), 3.36 (d, 1H), 3.25-3.20 (m, 1H), 3.05-3.01 (m, 1H), 2.41-1.88 (m, 9H), 1.50 (t, 3H). |
| 175a | 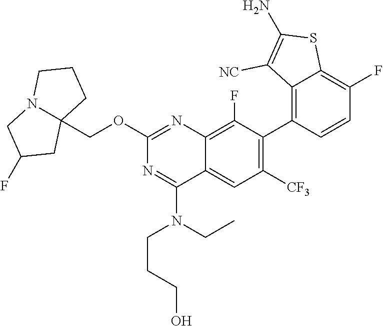 [SMILES: CCN(CCCO)c1nc(OCC23CCCN2CC(F)C3)nc2c(F)c(-c3ccc(F)c4sc(N)c(C#N)c34)c(C(F)(F)F)cc12] | |
| 176 | 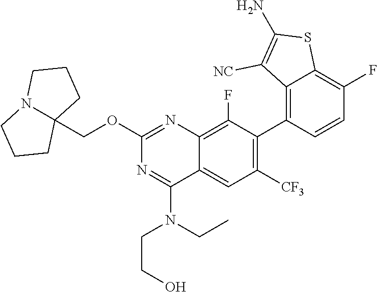 [SMILES: CCN(CCO)c1nc(OCC23CCCN2CCC3)nc2c(F)c(-c3ccc(F)c4sc(N)c(C#N)c34)c(C(F)(F)F)cc12] | LCMS m/z [M + 1]: 633.0 1H NMR (400 MHz, CD3OD) δ 8.51 (s, 1H), 7.24- 7.19 (m, 1H), 7.05-6.99 (m, 1H), 5.39-5.28 (m, 1H), 4.60 (s, 1H), 4.45-4.33 (m, 2H), 4.08-3.86 (m, 6H), 2.97-2.87 (m, 2H), 2.22-1.81 (m, 10H), 1.65-1.55 (m, 1H), 1.54-1.45 (m, 3H). |
| 177 | 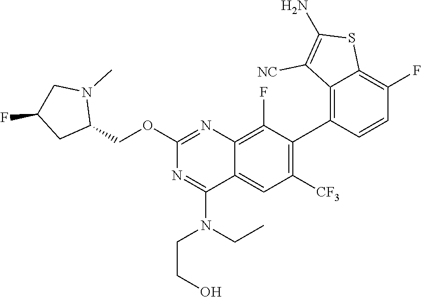 [SMILES: CCN(CCO)c1nc(OC[C@@H]2C[C@@H](F)CN2C)nc2c(F)c(-c3ccc(F)c4sc(N)c(C#N)c34)c(C(F)(F)F)cc12] | LCMS m/z [M + 1]: 625.0 1H NMR (400 MHz, CD3OD) δ 8.51 (s, 1H), 7.23- 7.20 (m, 1H), 7.05-6.99 (m, 1H), 5.29 (s, 0.61H), 5.15 (s, 0.61H), 4.61-4.51 (m, 3H), 4.03-3.91 (m, 6H), 3.64-3.47 (m, 2H), 2.86-2.76 (m, 1H), 2.65 (d, J = 2.8 Hz, 3H), 2.41-2.30 (m, 1H), 2.14-1.97 (m, 1H), 1.52-1.48 (m, 3H). |
| 177a | 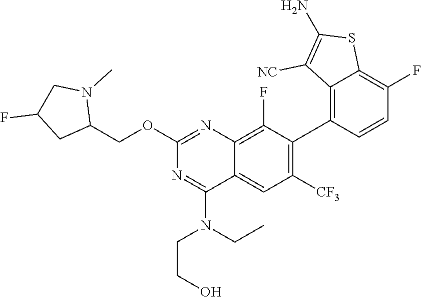 [SMILES: CCN(CCO)c1nc(OCC2CC(F)CN2C)nc2c(F)c(-c3ccc(F)c4sc(N)c(C#N)c34)c(C(F)(F)F)cc12] | |
| 178 | 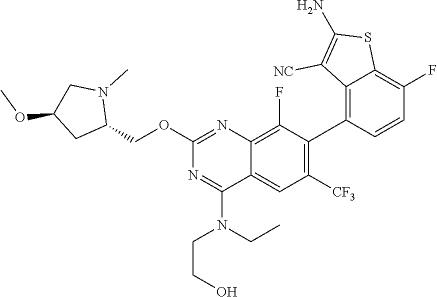 [SMILES: CCN(CCO)c1nc(OC[C@@H]2C[C@@H](OC)CN2C)nc2c(F)c(-c3ccc(F)c4sc(N)c(C#N)c34)c(C(F)(F)F)cc12] | LCMS m/z [M + 1]: 637.0 1H NMR (400 MHz, CD3OD) δ 8.53 (s, 1H), 7.23- 7.19 (m, 1H), 7.03-6.99 (m, 1H), 4.58-4.56 (m, 3H), 4.06-3.96 (m, 6H), 3.61-3.47 (m, 3H), 3.34 (s, 3H), 2.85-2.79 (m, 4H), 2.31-2.26 (m, 1H), 2.08-2.00 (m, 1H), 1.51-1.48 (m, 3H). |
| 178a | 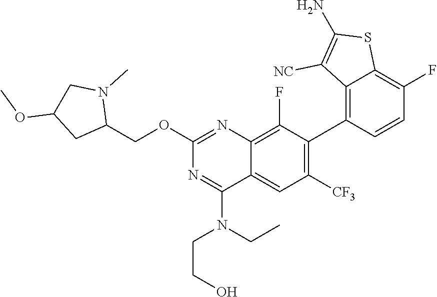 [SMILES: CCN(CCO)c1nc(OCC2CC(OC)CN2C)nc2c(F)c(-c3ccc(F)c4sc(N)c(C#N)c34)c(C(F)(F)F)cc12] | |
| 179 | 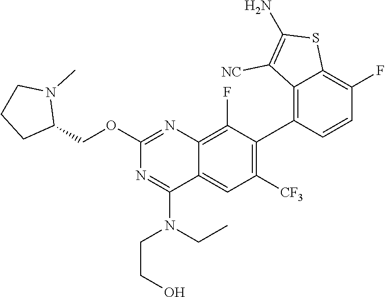 [SMILES: CCN(CCO)c1nc(OC[C@@H]2CCCN2C)nc2c(F)c(-c3ccc(F)c4sc(N)c(C#N)c34)c(C(F)(F)F)cc12] | LCMS m/z [M + 1]: 607.2 1H NMR (400 MHz, CD3OD) δ 8.55 (s, 1H), 7.27- 7.19 (m, 1H), 7.08-6.98 (m, 1H), 4.80-4.66 (m, 1H), 4.64-4.52 (m, 1H), 4.11-3.87 (m, 5H), 3.56-3.41 (m, 2H), 3.36-3.33 (m, 1H), 2.99-2.76 (m, 4H), 2.35-2.24 (m, 1H), 2.13-1.89 (m, 3H), 1.52 (t, J = 6.8 Hz, 3H). |
| 179a | 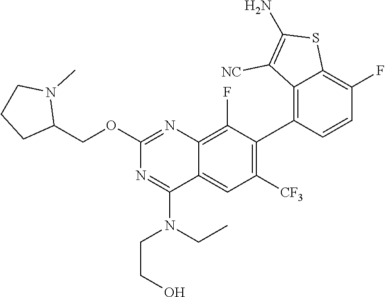 [SMILES: CCN(CCO)c1nc(OCC2CCCN2C)nc2c(F)c(-c3ccc(F)c4sc(N)c(C#N)c34)c(C(F)(F)F)cc12] | |
| 180 | 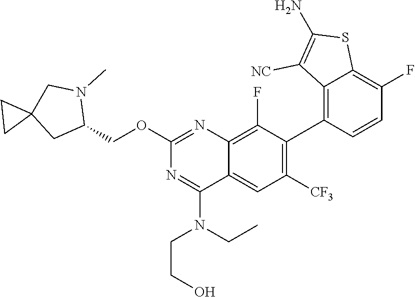 [SMILES: CCN(CCO)c1nc(OC[C@@H]2CC3(CC3)CN2C)nc2c(F)c(-c3ccc(F)c4sc(N)c(C#N)c34)c(C(F)(F)F)cc12] | LCMS m/z [M + 1]: 633.0 1H NMR (400 MHz, CD3OD) δ 8.56-8.49 (m, 1H), 7.25-7.17 (m, 1H), 7.06-6.97 (m, 1H), 4.70-4.54 (m, 2H), 4.06-3.90 (m, 5H), 3.50-3.38 (m, 1H), 3.09-2.57 (m, 6H), 2.31-2.19 (m, 1H), 1.86-1.75 (m, 1H), 1.58- 1.37 (m, 3H), 0.74-0.51 (m, 4H). |
| 180a | 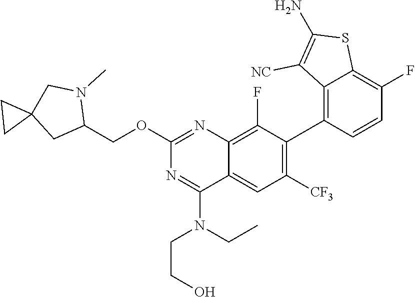 [SMILES: CCN(CCO)c1nc(OCC2CC3(CC3)CN2C)nc2c(F)c(-c3ccc(F)c4sc(N)c(C#N)c34)c(C(F)(F)F)cc12] | |
| 181 | 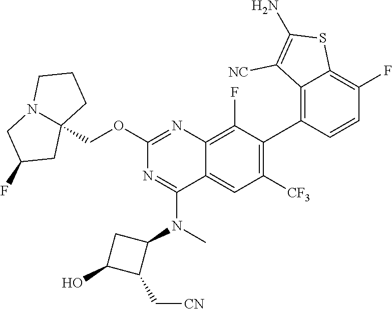 | LCMS m/z [M + 1]: 693.1 1H NMR (400 MHz, CD3OD) δ 8.31 (s, 1H), 7.24- 7.20 (m, 1H), 7.04-7.00 (m, 1H), 5.53 (s, 0.55H), 5.40 (s, 0.56 H), 4.61-4.55 (m, 1H), 4.50-4.41 (m, 2H), 3.93-3.59 (m, 7H), 3.51 (m, 3H), 2.79-2.03 (m, 10H). |
| 181a | 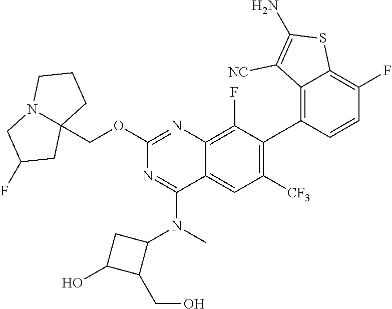 [SMILES: CN(c1nc(OCC23CCCN2CC(F)C3)nc2c(F)c(-c3ccc(F)c4sc(N)c(C#N)c34)c(C(F)(F)F)cc12)C1CC(O)C1CO] | |
| 182 | 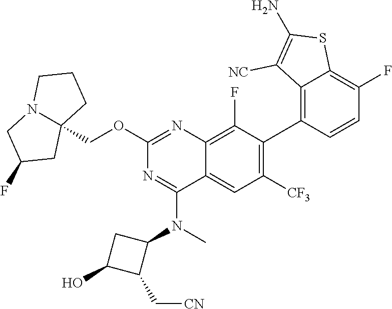 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@@H]1C[C@H](O)[C@H]1CC#N] | LCMS m/z [M + 1]: 702.1 1H NMR (400 MHz, CD3OD) δ 8.29 (s, 1H), 7.24- 7.20 (m, 1H), 7.04-7.00 (m, 1H), 5.48 (s, 0.56H), 5.35 (s, 0.57H), 4.51-4.43 (m, 3H), 3.93-3.86 (m, 1H), 3.65-3.42 (m, 6H), 3.23 (s, 1H), 2.97-2.01 (m, 13H). |
| 182a | 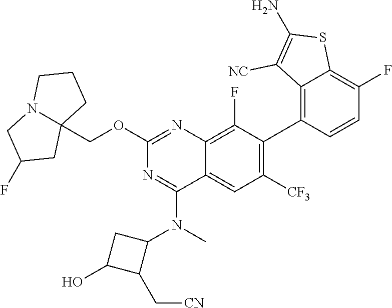 [SMILES: CN(c1nc(OCC23CCCN2CC(F)C3)nc2c(F)c(-c3ccc(F)c4sc(N)c(C#N)c34)c(C(F)(F)F)cc12)C1CC(O)C1CC#N] | |
| 183 | 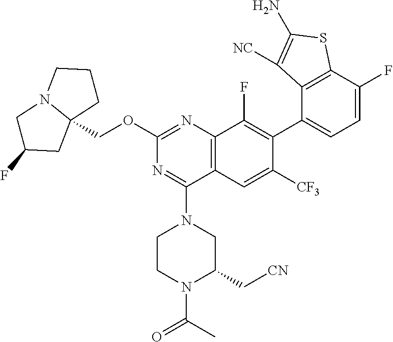 [SMILES: CC(=O)N1CCN(c2nc(OC[C@@]34CCCN3C[C@H](F)C4)nc3c(F)c(-c4ccc(F)c5sc(N)c(C#N)c45)c(C(F)(F)F)cc23)C[C@@H]1CC#N] | LCMS m/z [M + 1]: 729.1 1H NMR (400 MHz, CD3OD) δ 8.20 (d, 1H), 7.21 (s, 1H), 7.01 (t, 1H), 5.40 (s, 0.5H), 5.27 (s, 0.5H), 5.00 (s, 1H), 4.65-4.25 (m, 5H), 4.00-3.65 (m, 4H), 3.20-2.80 (m, 5H), 2.45-2.14 (m, 6H), 2.07-1.89 (m, 3H). |
| 184 | 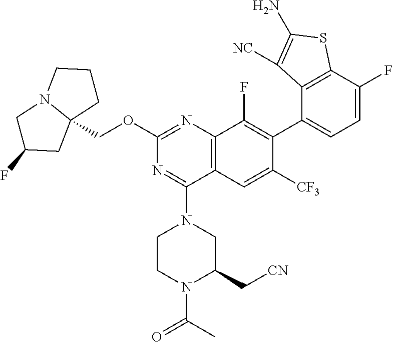 [SMILES: CC(=O)N1CCN(c2nc(OC[C@@]34CCCN3C[C@H](F)C4)nc3c(F)c(-c4ccc(F)c5sc(N)c(C#N)c45)c(C(F)(F)F)cc23)C[C@H]1CC#N] | LCMS m/z [M + 1]: 729.2 1H NMR (400 MHz, CD3OD) δ 8.19 (d, 1H), 7.21 (t, 1H), 7.01 (t, 1H), 5.40 (s, 0.5H), 5.26 (s, 0.5H), 5.07-4.96 (m, 1H), 4.72-4.28 (m, 5H), 4.02-3.35 (m, 5H), 3.23-2.87 (m, 4H), 2.45-2.14 (m, 6H), 2.06-1.91 (m, 3H). |
| 184a | 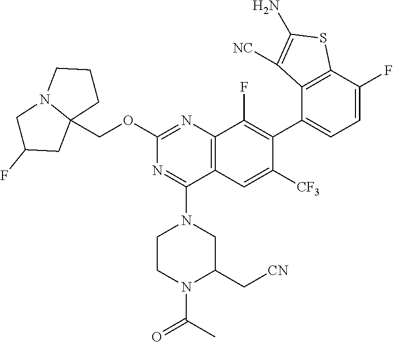 [SMILES: CC(=O)N1CCN(c2nc(OCC34CCCN3CC(F)C4)nc3c(F)c(-c4ccc(F)c5sc(N)c(C#N)c45)c(C(F)(F)F)cc23)CC1CC#N] | |
| 185 | 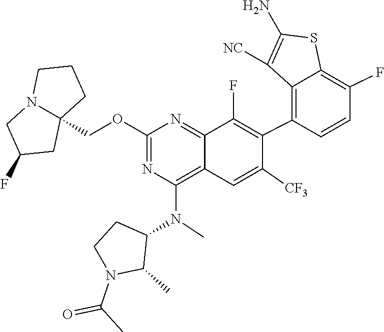 [SMILES: CC(=O)N1CC[C@H](N(C)c2nc(OC[C@@]34CCCN3C[C@H](F)C4)nc3c(F)c(-c4ccc(F)c5sc(N)c(C#N)c45)c(C(F)(F)F)cc23)[C@@H]1C] | |
| 185a | 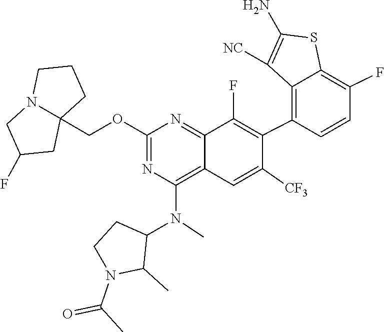 [SMILES: CC(=O)N1CCC(N(C)c2nc(OCC34CCCN3CC(F)C4)nc3c(F)c(-c4ccc(F)c5sc(N)c(C#N)c45)c(C(F)(F)F)cc23)C1C] | |
| 186 | 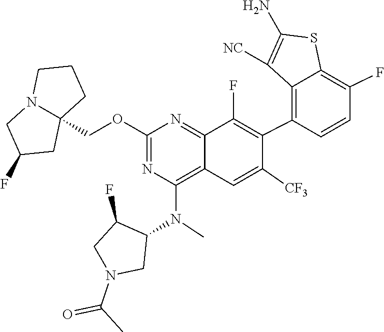 [SMILES: CC(=O)N1C[C@@H](F)[C@H](N(C)c2nc(OC[C@@]34CCCN3C[C@H](F)C4)nc3c(F)c(-c4ccc(F)c5sc(N)c(C#N)c45)c(C(F)(F)F)cc23)C1] | LCMS m/z [M + 1]: 722.1 1H NMR (400 MHz, CD3OD) δ 8.27 (s, 1H), 7.12 (m, 1H), 6.91 (t, 1H), 5.62 (s, 0.5H), 5.50 (s, 0.5H), 5.29 (s, 0.5H), 5.23-5.25 (m, 1H), 5.15 (s, 0.5H), 4.51 (s, 1H), 3.96-4.24 (m, 3H), 3.53-3.85 (m, 2H), 3.39-3.43 (m, 3H), 3.02-3.13 (m, 3H), 2.88-2.97 (m, 1H), 2.09 (t, 2H), 2.04 (s, 3H), 1.88-1.95 (m, 4H). |
| 187 | 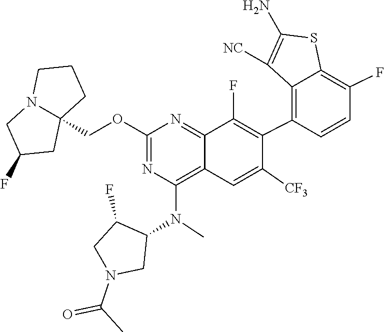 [SMILES: CC(=O)N1C[C@@H](N(C)c2nc(OC[C@@]34CCCN3C[C@H](F)C4)nc3c(F)c(-c4ccc(F)c5sc(N)c(C#N)c45)c(C(F)(F)F)cc23)[C@@H](F)C1] | LCMS m/z [M + 1]: 722.1. 1H NMR (400 MHz, CD3OD) δ 8.35 (d, 1H), 7.22 (t, 1H), 7.01 (t, 1H), 5.80 (d, 0.5H), 5.66 (d, 0.5H), 5.36 (s, 0.5H), 5.23 (s, 0.5H), 4.28-4.15 (m, 3H), 4.04-3.80 (m, 3H), 3.63 (d, 3H), 3.24 (d, 3H), 3.06- 2.99 (m, 1H), 2.36-2.22 (m, 2H), 2.14 (d, 4H), 2.0.1- 1.87 (m, 4H). |
| 187a | 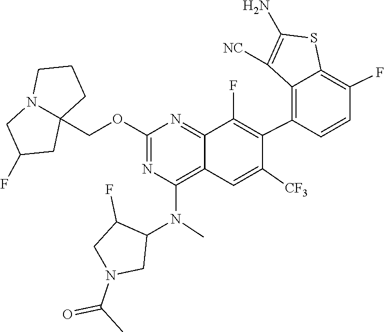 [SMILES: CC(=O)N1CC(F)C(N(C)c2nc(OCC34CCCN3CC(F)C4)nc3c(F)c(-c4ccc(F)c5sc(N)c(C#N)c45)c(C(F)(F)F)cc23)C1] | |
| 188 | 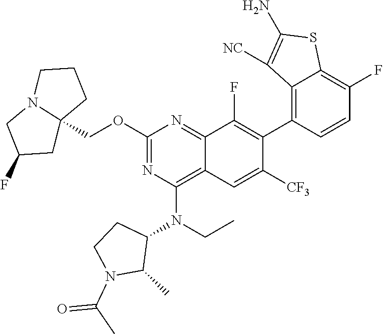 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@H]1CCN(C(C)=O)[C@H]1C] | |
| 189 | 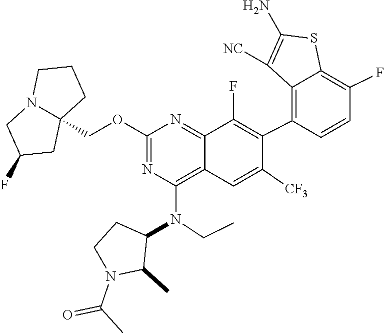 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@@H]1CCN(C(C)=O)[C@@H]1C] | LCMS m/z [M + 1]: 732.1 1H NMR (400 MHz, CDOD) δ 8.31 (s, 1H), 7.24- 7.21 (m, 1H), 7.03-6.98 (m, 1H), 5.38 (s, 0.5H), 5.24 (s, 0.5H), 4.98-4.94 (m, 1H), 4.79-4.71 (m, 1H), 4.37-4.25 (m, 2H), 4.06-4.01 (m, 1H), 3.97-3.87 (m, 1H), 3.81 (t, 1H), 3.70-3.46 (m, 2H), 3.27-3.19 (m, 2H), 3.07-2.98 (m, 1H), 2.67-2.54 (m, 1H), 2.53-2.44 (m, 2H), 2.14-1.80 (m, 7H), 1.44-1.33 (m, 3H), 1.11- 1.97 (m, 3H) |
| 189a | 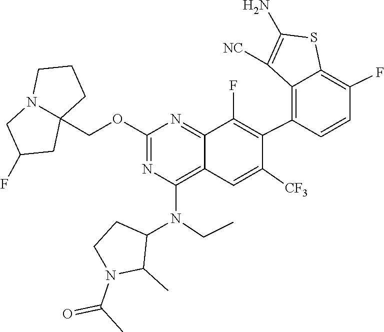 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(C)=O)C1C] | |
| 190 | 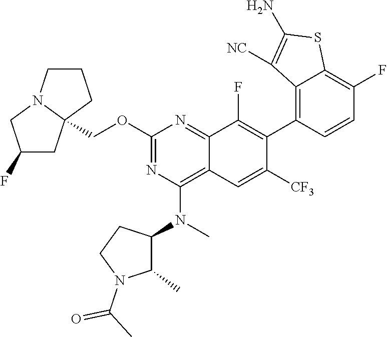 [SMILES: CC(=O)N1CC[C@@H](N(C)c2nc(OC[C@@]34CCCN3C[C@H](F)C4)nc3c(F)c(-c4ccc(F)c5sc(N)c(C#N)c45)c(C(F)(F)F)cc23)[C@@H]1C] | LCMS m/z [M + 1]: 718.3 1H NMR (400 MHz, CD3OD) δ 8.29 (s, 1H), 7.21 (t, 1H), 7.00 (t, 1H), 5.38 (s, 0.5H), 5.24 (s, 0.5H), 5.05- 5.02 (m, 1H), 4.38-4.24 (m, 3H), 3.99-3.87 (m, 1H), 3.75-3.88 (m, 1H), 3.48-3.36 (m, 3H), 3.27-3.23 (m, 2H), 3.04-3.00 (m, 1H), 2.59-2.54 (m, 1H), 2.38-2.11 (m, 7H), 2.01-1.90 (m, 3H), 1.48-1.37 (m, 3H). |
| 191 | 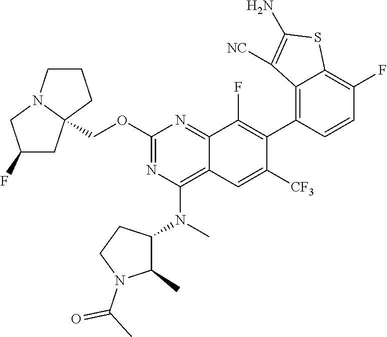 [SMILES: CC(=O)N1CC[C@H](N(C)c2nc(OC[C@@]34CCCN3C[C@H](F)C4)nc3c(F)c(-c4ccc(F)c5sc(N)c(C#N)c45)c(C(F)(F)F)cc23)[C@H]1C] | LCMS m/z [M + 1]: 718.1 1H NMR (400 MHz, CD3OD) δ 8.32 (s, 1H), 7.24 (t, 1H), 7.03 (t, 1H), 5.41 (s, 0.5H), 5.27 (s, 0.5H), 5.10- 5.04 (m, 1H), 4.38-4.26 (m, 3H), 3.96-3.89 (m, 1H), 3.77-3.71 (m, 1H), 3.56-3.36 (m, 4H), 3.29-3.18 (m, 2H), 3.12-3.04 (m, 1H), 2.60-2.58 (m, 1H), 2.40-2.37 (m, 3H), 2.28-1.91 (m, 7H), 1.50-1.39 (m, 3H). |
| 192 | 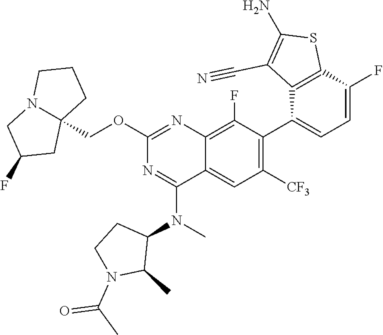 | LCMS m/z [M + 1]: 718.1 1H NMR (400 MHz, CD3OD) δ 8.41 (s, 1H), 7.22 (t, 1H), 7.03 (t, 1H), 5.62 (s, 0.5H), 5.49 (s, 0.5H), 4.98 (t, 1H), 4.83-4.66 (m, 3H), 4.02-3.80 (m, 4H), 3.71- 3.44 (m, 5H), 2.77-2.58 (m, 3H), 2.49-2.30 (m, 4H), 2.20-2.06 (m, 4H), 1.18-1.07 (m, 3H). |
| 193 | 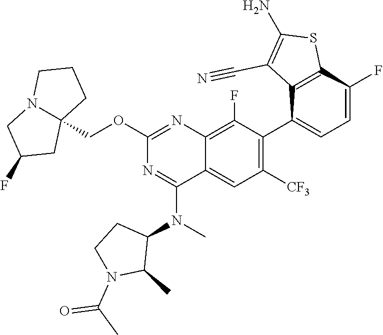 | LCMS m/z [M + 1]: 718.1 1H NMR (400 MHz, CD3OD) δ 8.41 (s, 1H), 7.22 (t, 1H), 7.03 (t, 1H), 5.63 (s, 0.5H), 5.50 (s, 0.5H), 5.00 (t, 1H), 4.83-4.60 (m, 3H), 4.08-3.81 (m, 4H), 3.77- 3.57 (m, 4H), 3.48-3.42 (m, 1H), 2.77-2.58 (m, 3H), 2.49-2.30 (m, 4H), 2.20-2.03 (m, 4H), 1.19-1.07 (m, 3H). |
| 194 | 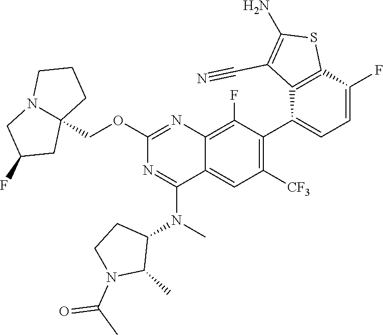 | LCMS m/z [M + 1]: 718.1 1H NMR (400 MHz, CD3OD) δ 8.41 (s, 1H), 7.24 (t, 1H), 7.03 (t, 1H), 5.62 (s, 0.5H), 5.49 (s, 0.5H), 5.00 (t, 1H), 4.80-4.63 (m, 3H), 4.05-3.81 (m, 4H), 3.71- 3.65 (m, 4H), 3.49-3.43 (m, 1H), 2.76-2.56 (m, 3H), 2.49-2.30 (m, 4H), 2.16-2.09 (m, 4H), 1.17-1.05 (m, 3H). |
| 195 | 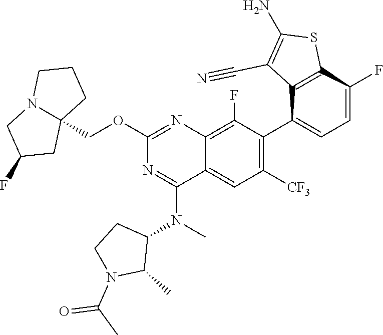 | LCMS m/z [M + 1]: 718.1 1H NMR (400 MHz, CD3OD) δ 8.41 (s, 1H), 7.23 (t, 1H), 7.03 (t, 1H), 5.62 (s, 0.5H), 5.49 (s, 0.5H), 4.98 (t, 1H), 4.81-4.58 (m, 3H), 4.04-3.80 (m, 4H), 3.71- 3.56 (m, 4H), 3.49-3.42 (m, 1H), 2.73-2.57 (m, 3H), 2.49-2.30 (m, 4H), 2.14-2.09 (m, 4H), 1.16-1.05 (m, 3H). |
| 195a | 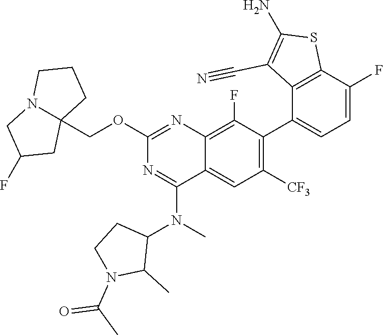 [SMILES: CC(=O)N1CCC(N(C)c2nc(OCC34CCCN3CC(F)C4)nc3c(F)c(-c4ccc(F)c5sc(N)c(C#N)c45)c(C(F)(F)F)cc23)C1C] | |
| 196 | 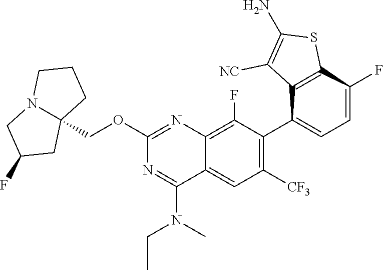 | LCMS m/z [M + 1]: 621.0. 1H NMR (400 MHz, CD3OD) δ 8.29 (s, 1H), 7.22- 7.18 (m, 1H), 7.00 (t, 1H), 5.35 (s, 0.5H), 5.22 (s, 0.5H), 4.31-4.19 (m, 2H), 3.96-3.82 (m, 2H), 3.62 (s, 3H), 3.24-3.13 (m, 3H), 3.02-2.96 (m, 1H), 2.36-2.11 (m, 3H), 1.98-1.85 (m, 3H), 1.44 (t, 3H). |
| 197 | 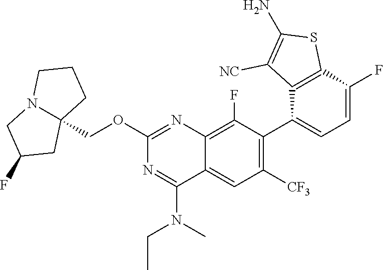 | LCMS m/z [M + 1]: 621.0. ¹H NMR (400 MHz, CD3OD) δ 8.50 (s, 1H), 7.22- 7.19 (m, 1H), 7.00 (t, 1H), 5.36 (s, 0.5H), 5.22 (s, 0.5H), 4.31-4.20 (m, 2H), 3.93-3.82 (m, 2H), 3.49 (s, 3H), 3.22-3.17 (m, 3H), 3.03-2.97 (m, 1H), 2.38-2.10 (m, 3H), 2.03-1.82 (m, 3H), 1.44 (t, 3H). |
| 197a | 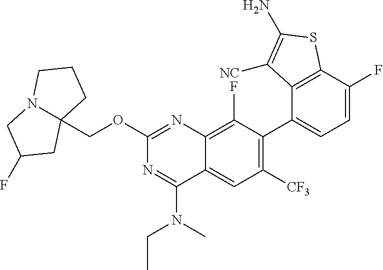 [SMILES: CCN(C)c1nc(OCC23CCCN2CC(F)C3)nc2c(F)c(-c3ccc(F)c4sc(N)c(C#N)c34)c(C(F)(F)F)cc12] | |
| 198 | 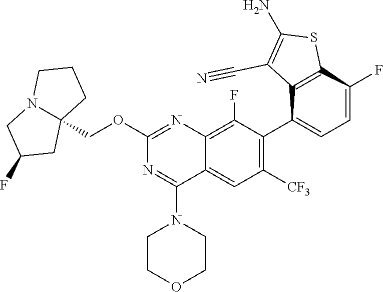 | LCMS m/z [M + 1]: 649.0. 1H NMR (400 MHz, DMSO) δ 8.09 (d, 3H), 7.23- 7.26 (m, 1H), 7.13 (t, 1H), 5.35 (s, 0.5H), 5.21 (s, 0.5H), 4.15 (d, 1H), 4.05 (d, 1H), 3.93-3.96 (m, 2H), 3.78-3.88 (m, 6H), 3.03-3.11 (m, 3H), 2.82-2.84 (m, 1H), 2.01-2.16 (m, 3H), 1.77-1.86 (m, 3H). |
| 199 | 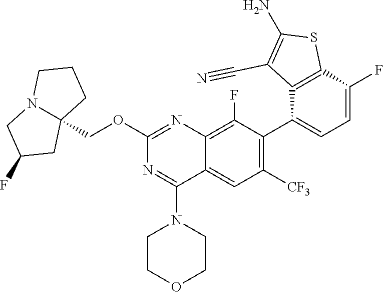 | LCMS m/z [M + 1]: 649.0 1H NMR (400 MHz, DMSO) δ 8.09 (d, 3H), 7.23- 7.26 (m, 1H), 7.13 (t, 1H), 5.34 (s, 0.5H), 5.21 (s, 0.5H), 4.14 (d, 1H), 4.06 (d, 1H), 3.93-3.96 (m, 2H), 3.78-3.82 (m, 6H), 3.02-3.10 (m, 3H), 2.82-2.83 (m, 1H), 2.01-2.15 (m, 3H), 1.77-1.85 (m, 3H). |
| 199a | 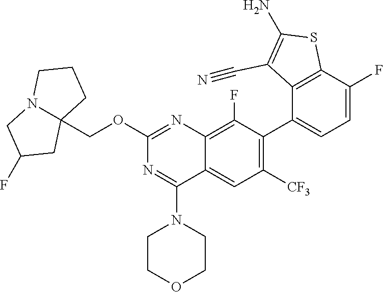 [SMILES: N#Cc1c(N)sc2c(F)ccc(-c3c(C(F)(F)F)cc4c(N5CCOCC5)nc(OCC56CCCN5CC(F)C6)nc4c3F)c12] | |
| 200 | 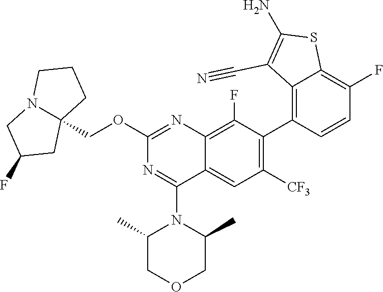 [SMILES: C[C@H]1COC[C@H](C)N1c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 677.3 1H NMR (400 MHz, CD3OD) δ 8.19 (s, 1H), 7.19- 7.25 (m, 1H), 6.98-7.03 (m, 1H), 5.37 (s, 0.5H), 5.24 (s, 0.5H), 4.33 (s, 2H), 4.02-4.10 (m, 4H), 3.63-3.69 (m, 2H), 3.18-3.24 (m, 3H), 2.98-3.04 (m, 1H), 2.22- 2.39 (m, 2H), 2.14-2.18 (m, 1H), 1.89-2.04 (m, 3H), 1.12-1.17 (m, 6H). |
| 201 | 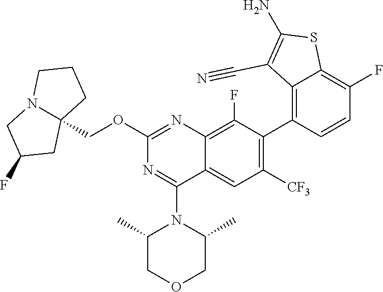 [SMILES: C[C@@H]1COC[C@H](C)N1c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 677.0 1H NMR (400 MHz, CD3OD) δ 8.28 (s, 1H), 7.22- 7.19 (m, 1H), 7.00 (t, 1H), 5.37 (s, 0.5H), 5.24 (s, 0.5H), 4.85-4.75 (m, 2H), 4.32-4.20 (m, 2H), 3.86- 3.73 (m, 4H), 3.28-3.19 (m, 3H), 3.05-2.99 (m, 1H), 2.35-2.12 (m, 3H), 2.03-1.87 (m, 3H), 1.66-1.63 (m, 6H) |
| 201a | 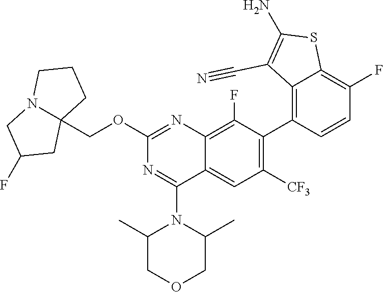 [SMILES: CC1COCC(C)N1c1nc(OCC23CCCN2CC(F)C3)nc2c(F)c(-c3ccc(F)c4sc(N)c(C#N)c34)c(C(F)(F)F)cc12] | |
| 202 | 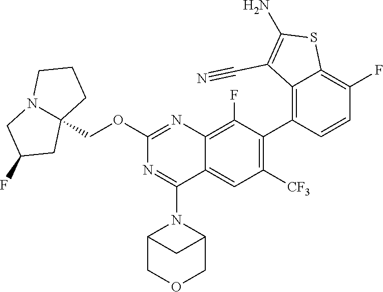 [SMILES: N#Cc1c(N)sc2c(F)ccc(-c3c(C(F)(F)F)cc4c(N5C6COCC5C6)nc(OC[C@@]56CCCN5C[C@H](F)C6)nc4c3F)c12] | LCMS m/z [M + 1]: 661.0 1H NMR (400 MHz, CD3OD) δ 8.00 (s, 1H), 7.22- 7.19 (m, 1H), 7.00 (t, 1H), 5.37 (s, 0.5H), 5.23 (s, 0.5H), 5.14-4.98 (m, 2H), 4.53-4.33 (m, 2H), 4.30- 4.19 (m, 2H), 4.00 (d, 2H), 3.25-3.18 (m, 2H), 3.07- 2.95 (m, 2H), 2.31-1.88 (m, 8H) |
| 202a | 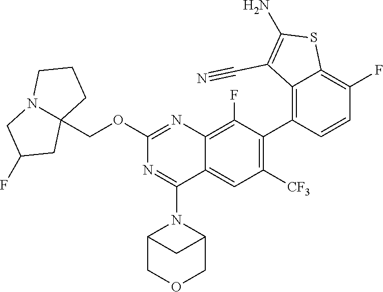 [SMILES: N#Cc1c(N)sc2c(F)ccc(-c3c(C(F)(F)F)cc4c(N5C6COCC5C6)nc(OCC56CCCN5CC(F)C6)nc4c3F)c12] | |
| 203 | 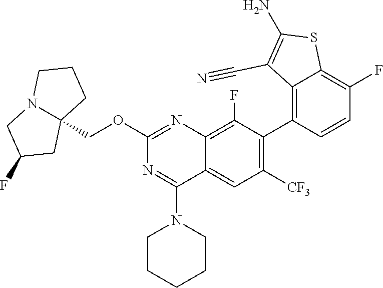 [SMILES: N#Cc1c(N)sc2c(F)ccc(-c3c(C(F)(F)F)cc4c(N5CCCCC5)nc(OC[C@@]56CCCN5C[C@H](F)C6)nc4c3F)c12] | LCMS m/z [M + 1]: 647.0 1H NMR (400 MHz, CD3OD) δ 8.09 (s, 1H), 7.19- 7.22 (m, 1H), 7.00 (t, 1H), 5.36 (s, 0.5H), 5.23 (s, 0.5H), 4.29-4.33 (m, 1H), 4.20-4.24 (m, 1H), 3.92 (s, 3H), 3.15-3.25 (m, 4H), 3.00 (s, 1H), 2.34-2.37 (m, 1H), 2.12-2.26 (m, 3H), 1.95-1.99 (m, 2H), 1.87-1.93 (m, 1H), 1.83 (s, 5H). |
| 203a | 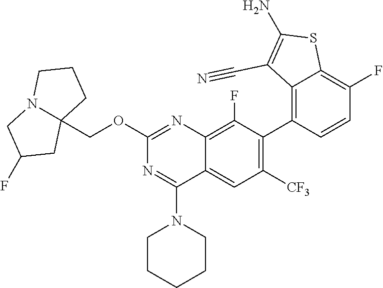 [SMILES: N#Cc1c(N)sc2c(F)ccc(-c3c(C(F)(F)F)cc4c(N5CCCCC5)nc(OCC56CCCN5CC(F)C6)nc4c3F)c12] | |
| 204 | 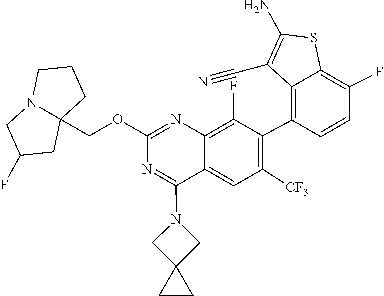 [SMILES: N#Cc1c(N)sc2c(F)ccc(-c3c(C(F)(F)F)cc4c(N5CC6(CC6)C5)nc(OCC56CCCN5CC(F)C6)nc4c3F)c12] | LCMS m/z [M + 1]: 645.2 1H NMR (400 MHz, CD3OD) δ 8.07 (s, 1H), 7.18- 7.21 (m, 1H), 6.98-7.02 (m, 1H), 5.41 (s, 0.5H), 5.28 (s, 0.5H), 4.48-4.82 (m, 4H), 4.37-4.39 (m, 1H), 4.28-4.32 (m, 1H), 3.43-3.51 (m, 1H), 3.35-3.40 (m, 2H), 3.07-3.13 (m, 1H), 2.14-2.37 (m, 3H), 1.93-2.07 (m, 3H), 0.84 (s, 4H). |
| 204a | 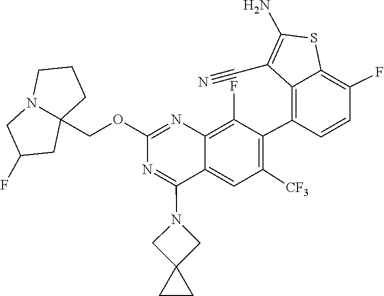 [SMILES: N#Cc1c(N)sc2c(F)ccc(-c3c(C(F)(F)F)cc4c(N5CC6(CC6)C5)nc(OCC56CCCN5CC(F)C6)nc4c3F)c12] | |
| 205 | 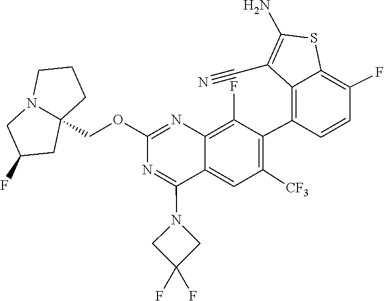 [SMILES: N#Cc1c(N)sc2c(F)ccc(-c3c(C(F)(F)F)cc4c(N5CC(F)(F)C5)nc(OC[C@@]56CCCN5C[C@H](F)C6)nc4c3F)c12] | LCMS m/z [M + 1]: 655.2 1H NMR (400 MHz, CD3OD) δ 8.01 (s, 1H), 7.21- 7.18 (m, 1H), 7.03-6.99 (m, 1H), 5.34 (s, 0.68H), 5.24 (s, 0.59H), 5.07-5.01 (m, 4H), 4.34-4.23 (m, 2H), 3.26-2.99 (m, 6H), 2.38-2.12 (m, 3H), 2.03-1.88 (m, 3H). |
| 205a | 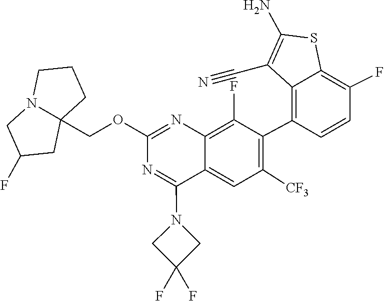 [SMILES: N#Cc1c(N)sc2c(F)ccc(-c3c(C(F)(F)F)cc4c(N5CC(F)(F)C5)nc(OCC56CCCN5CC(F)C6)nc4c3F)c12] | |
| 206 | 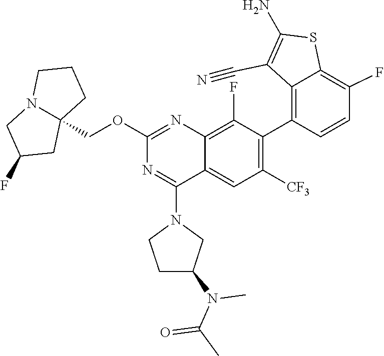 [SMILES: CC(=O)N(C)[C@H]1CCN(c2nc(OC[C@@]34CCCN3C[C@H](F)C4)nc3c(F)c(-c4ccc(F)c5sc(N)c(C#N)c45)c(C(F)(F)F)cc23)C1] | LCMS m/z [M + 1]: 704 1H NMR (400 MHz, CD3OD) δ 8.42 (s, 1H), 7.17- 7.24 (m, 1H), 7.00 (t, 1H), 5.38 (s, 0.5H), 5.24 (s, 0.5H), 4.21-4.41 (m, 3H), 3.92-4.21 (m, 3H), 3.22- 3.38 (m, 1H), 3.18-3.29 (m, 3H), 2.99-3.14 (m, 3H), 2.94 (d, 1H), 2.10-2.42 (m, 8H), 1.87-2.05 (m, 3H). |
| 207 | 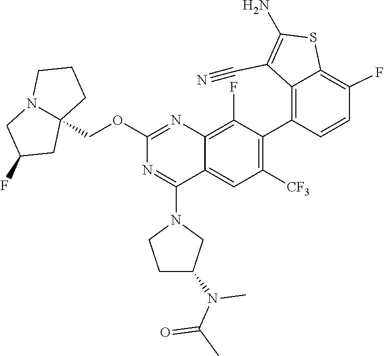 [SMILES: CC(=O)N(C)[C@@H]1CCN(c2nc(OC[C@@]34CCCN3C[C@H](F)C4)nc3c(F)c(-c4ccc(F)c5sc(N)c(C#N)c45)c(C(F)(F)F)cc23)C1] | LCMS m/z [M + 1]: 704.1 1H NMR (400 MHz, CD3OD) δ 8.41 (s, 1H), 7.19 (t, 1H), 7.00 (t, 1H), 5.38 (s, 0.5H), 5.24 (s, 0.5H), 4.35- 4.10 (m, 5H), 4.03-3.92 (m, 1H), 3.29-3.19 (m, 3H), 3.09-2.97 (m, 3H), 2.94 (s, 1H), 2.38-2.11 (m, 8H), 2.06-1.82 (m, 4H). |
| 207a | 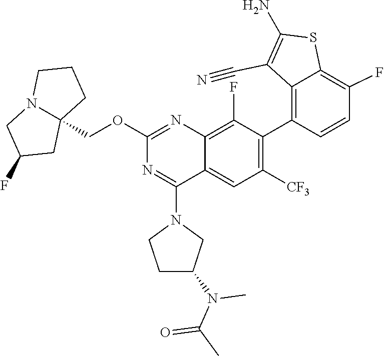 [SMILES: CC(=O)N(C)[C@@H]1CCN(c2nc(OC[C@@]34CCCN3C[C@H](F)C4)nc3c(F)c(-c4ccc(F)c5sc(N)c(C#N)c45)c(C(F)(F)F)cc23)C1] | |
| 208 | 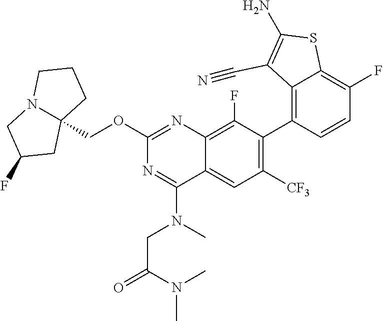 [SMILES: CN(C)C(=O)CN(C)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 678.1 1H NMR (400 MHz, CD3OD) δ 8.287 (s, 1H), 7.23- 7.19 (m, 1H), 7.008 (t, 1H), 5.393 (s, 0.5H), 5.258 (s, 0.5H), 4.803 (t, 1H), 4.66-4.59 (m, 1H), 4.29 (d, 1H), 4.204 (d, 1H), 3.61 (s, 3H), 3.45-3.36 (m, 1H), 3.28- 3.23 (m, 2H), 3.168 (s, 3H), 3.09-3.05 (m, 1H), 3.036 (s, 3H), 2.40-2.17 (m, 3H), 2.10-1.93 (m, 3H). |
| 208a | 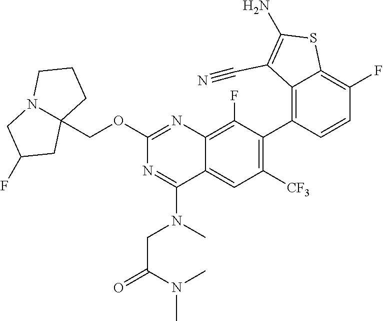 [SMILES: CN(C)C(=O)CN(C)c1nc(OCC23CCCN2CC(F)C3)nc2c(F)c(-c3ccc(F)c4sc(N)c(C#N)c34)c(C(F)(F)F)cc12] | |
| 209 | 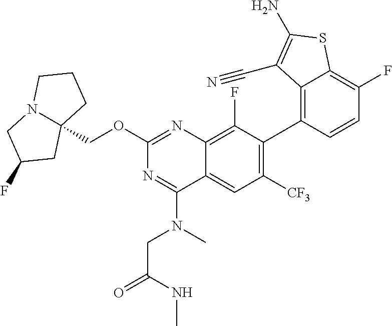 [SMILES: CNC(=O)CN(C)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 664.0 1H NMR (400 MHz, CD3OD) δ 8.38 (s, 1H), 7.19- 7.22 (m, 1H), 7.01 (t, 1H), 5.37 (s, 0.5H), 5.24 (s, 0.5H), 4.60 (s, 1H), 4.37-4.48 (m, 2H), 4.17-4.27 (m, 2H), 3.63 (s, 2H), 3.17-3.27 (m, 3H), 2.98-3.03 (m, 1H), 2.80 (s, 3H), 2.09-2.36 (m, 3H), 1.87-2.03 (m, 3H). |
| 209a | 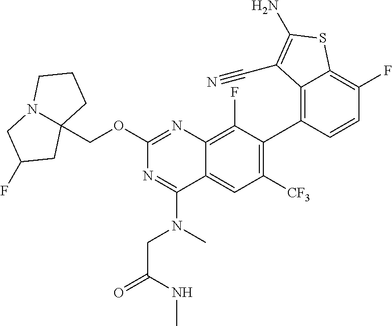 [SMILES: CNC(=O)CN(C)c1nc(OCC23CCCN2CC(F)C3)nc2c(F)c(-c3ccc(F)c4sc(N)c(C#N)c34)c(C(F)(F)F)cc12] | |
| 210 | 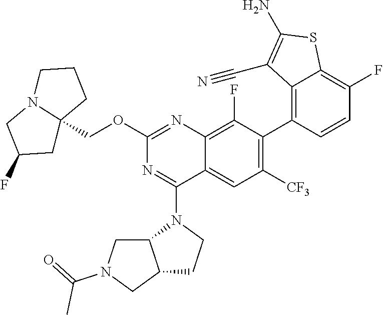 [SMILES: CC(=O)N1C[C@H]2CCN(c3nc(OC[C@@]45CCCN4C[C@H](F)C5)nc4c(F)c(-c5ccc(F)c6sc(N)c(C#N)c56)c(C(F)(F)F)cc34)[C@H]2C1] | LCMS m/z [M + 1]: 716.1 1H NMR (400 MHz, CD3OD) δ 8.43 (s, 1H), 7.19- 7.22 (m, 1H), 7.00 (t, 1H), 5.39 (s, 0.5H), 5.28 (s, 0.5H), 5.01-5.15 (m, 1H), 4.16-4.47 (m, 4H), 3.87- 4.13 (m, 2H), 3.68-3.86 (m, 2H), 3.43-3.67 (m, 2H), 2.99-3.18 (m, 2H), 1.73-2.49 (m, 12H). |
| 211 | 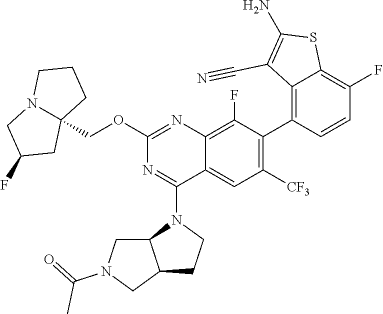 [SMILES: CC(=O)N1C[C@@H]2CCN(c3nc(OC[C@@]45CCCN4C[C@H](F)C5)nc4c(F)c(-c5ccc(F)c6sc(N)c(C#N)c56)c(C(F)(F)F)cc34)[C@@H]2C1] | LCMS m/z [M + 1]: 716.1. 1H NMR (400 MHz, CD3OD) δ 8.44 (s, 1H), 7.22- 7.19 (m, 1H), 7.01 (t, 1H), 5.43 (d, 0.5H), 5.30 (d, 0.5H), 5.12 (s, 0.5H), 5.02 (s, 0.5H), 4.47-4.21 (m, 4H), 4.06-3.72 (m, 3H), 3.65-3.36 (m, 3H), 3.23-3.08 (m, 2H), 2.49-1.93 (m, 12H). |
| 211a | 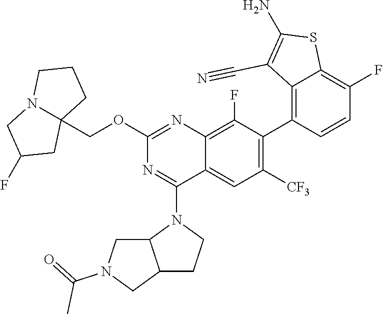 [SMILES: CC(=O)N1CC2CCN(c3nc(OCC45CCCN4CC(F)C5)nc4c(F)c(-c5ccc(F)c6sc(N)c(C#N)c56)c(C(F)(F)F)cc34)C2C1] | |

Table 6 includes selected compounds of the present disclosure.

TABLE 6
Selected compounds of the present disclosure.

| Cmpd. | | |
|---|---|---|
| No. | Structure | Analytical data |
| 212 | 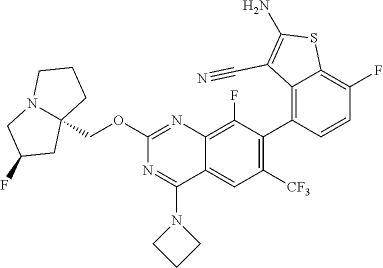 [SMILES: N#Cc1c(N)sc2c(F)ccc(-c3c(C(F)(F)F)cc4c(N5CCC5)nc(OC[C@@]56CCCN5C[C@H](F)C6)nc4c3F)c12] | LCMS m/z [M + 1]: 619.0 1H NMR (400 MHz, CD3OD) δ 8.09 (s, 1H), 7.18- 7.21 (m, 1H), 6.99-7.03 (m, 1H), 5.52-5.53 (m, 0.5H), 5.39-5.40 (m, 0.5H), 4.40-4.68 (m, 4H), 3.54- 3.94 (m, 4H), 3.18-3.29 (m, 2H), 2.57-2.65 (m, 2H), 2.38-2.55 (m, 2H), 2.29-2.35 (m, 1H), 2.17-2.25 (m, 2H), 1.99-2.08 (m, 1H). |
| 212a | 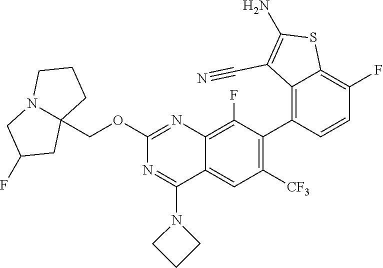 [SMILES: N#Cc1c(N)sc2c(F)ccc(-c3c(C(F)(F)F)cc4c(N5CCC5)nc(OCC56CCCN5CC(F)C6)nc4c3F)c12] | |
| 214 | 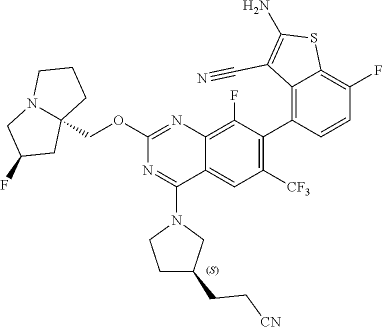 | LCMS m/z [M + 1]: 686.1 1H NMR (400 MHz, CD3OD) δ 8.43 (d, J = 4.8 Hz, 1H), 7.21 (d, J = 5.6 Hz, 1H), 7.03-6.96 (m, 1H), 5.39- 5.33 (m, 0.58H), 5.26 (s, 0.58H), 4.38-4.35 (m, 1H), 4.24-4.08 (m, 4H), 3.70 (s, 1H), 3.48-3.38 (m, 1H), 3.08-3.20 (m, 2H), 3.06-3.02 (m, 1H), 2.66-2.57 (m, 2H), 2.50-2.14 (m, 6H), 2.05-1.84 (m, 7H). |
| 214a | 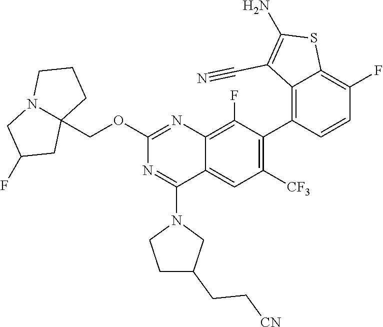 [SMILES: N#CCCC1CCN(c2nc(OCC34CCCN3CC(F)C4)nc3c(F)c(-c4ccc(F)c5sc(N)c(C#N)c45)c(C(F)(F)F)cc23)C1] | |
| 215 | 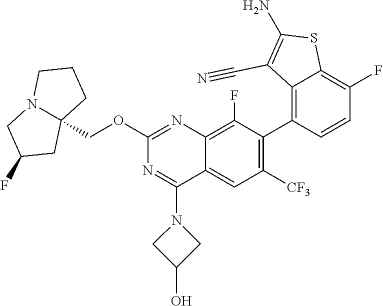 [SMILES: N#Cc1c(N)sc2c(F)ccc(-c3c(C(F)(F)F)cc4c(N5CC(O)C5)nc(OC[C@@]56CCCN5C[C@H](F)C6)nc4c3F)c12] | LCMS m/z [M + 1]: 635.0 1H NMR (400 MHz, CD3OD) δ 8.05 (s, 1H), 7.17- 7.20 (m, 1H), 6.95-7.02 (m, 1H), 5.38 (s, 0.5H), 5.25 (s, 0.5H), 4.80-4.81(m, 3H), 4.28-4.52 (m, 3H), 4.22- 4.26 (m, 1H), 3.36-3.40 (m, 1H), 3.22-3.27 (m, 2H), 3.01-3.05 (m, 1H), 2.13-2.36 (m, 3H), 1.89-2.03 (m, 3H). |
| 215a | 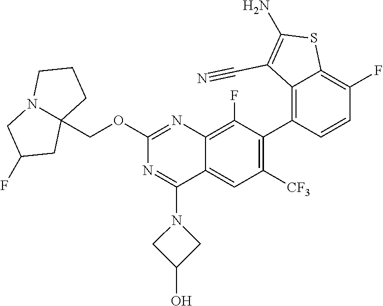 [SMILES: N#Cc1c(N)sc2c(F)ccc(-c3c(C(F)(F)F)cc4c(N5CC(O)C5)nc(OCC56CCCN5CC(F)C6)nc4c3F)c12] | |
| 216 | 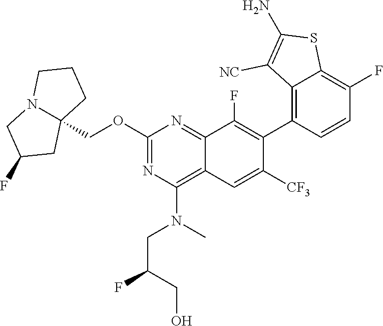 [SMILES: CN(C[C@H](F)CO)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 669.3 1H NMR (400 MHz, CD3OD) δ 8.46 (s, 1H), 7.25- 7.18 (m, 1H), 7.05-6.98 (m, 1H), 5.41-5.37 (s, 0.5H), 5.27-5.24 (s, 0.5H), 5.16-5.11 (m, 0.5H), 5.04-5.00 (m, 0.5H), 4.42-4.21 (m, 3H), 4.10-3.99 (m, 1H), 3.91-3.76 (m, 2H), 3.73-3.62 (m, 3H), 3.29-3.12 (m, 3H), 3.09-3.01 (m, 1H), 2.40-2.12 (m, 3H), 2.05-1.88 (m, 3H). |
| 216a | 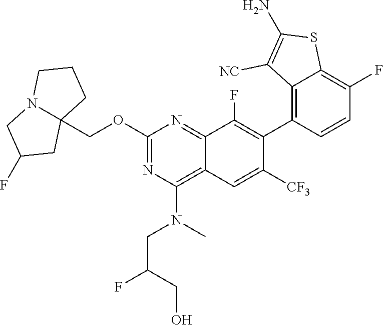 [SMILES: CN(CC(F)CO)c1nc(OCC23CCCN2CC(F)C3)nc2c(F)c(-c3ccc(F)c4sc(N)c(C#N)c34)c(C(F)(F)F)cc12] | |
| 217 | 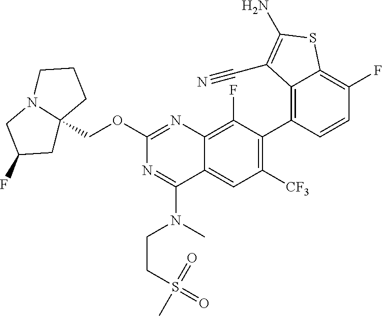 [SMILES: CN(CCS(C)(=O)=O)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 699.16 1H NMR (400 MHz, CD3OD) δ 8.38 (s, 1H), 7.24- 7.16 (m, 1H), 7.05-6.95 (m, 1H), 5.38 (s, 0.5H), 5.25 (s, 0.5H), 4.43-4.20 (m, 4H), 3.79-3.58 (m, 5H), 3.42-3.31 (m, 1H), 3.27-3.16 (m, 2H), 3.13-2.97 (m, 4H), 2.44-2.26 (m, 1H), 2.25-2.11 (m, 2H), 2.04-1.87 (m, 3H). |
| 217a | 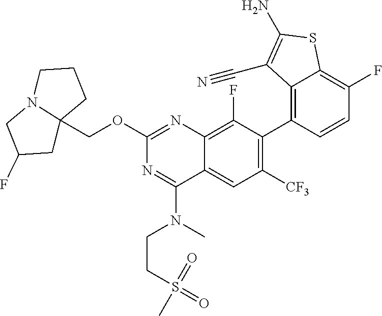 [SMILES: CN(CCS(C)(=O)=O)c1nc(OCC23CCCN2CC(F)C3)nc2c(F)c(-c3ccc(F)c4sc(N)c(C#N)c34)c(C(F)(F)F)cc12] | |
| 218 | 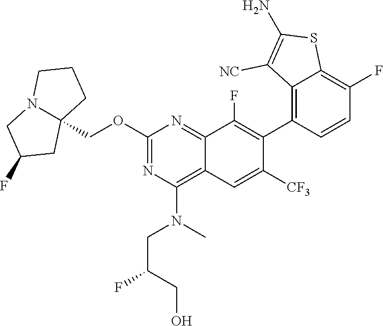 [SMILES: CN(C[C@@H](F)CO)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 669.18 1H NMR (400 MHz, CD3OD) δ 8.45 (s, 1H), 7.25- 7.15 (m, 1H), 7.05-6.95 (m, 1H), 5.37 (s, 0.5H), 5.23 (s, 0.5H), 5.15-5.09 (m, 0.5H), 5.04-4.97 (m, 0.5H), 4.43-4.16 (m, 3H), 4.11-3.96 (m, 1H), 3.92-3.73 (m, 2H), 3.73-3.58 (m, 3H), 3.28-3.13 (m, 3H), 3.07-2.95 (m, 3H), 2.41-2.07 (m, 3H), 2.05-1.82 (m, 3H). |
| 220 | 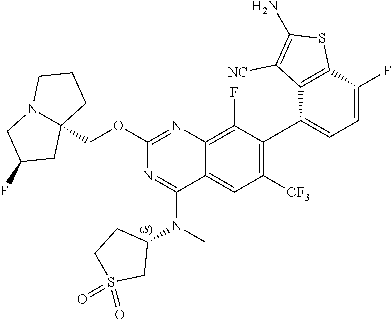 | LCMS m/z [M + 1]: 711.0 1H NMR (400 MHz, CD3OD) δ 8.32 (s, 1H), 7.25- 7.17 (m, 1H), 7.06-6.96 (m, 1H), 5.53-5.44 (m, 1H), 5.40-5.36 (m, 0.5H), 5.26-5.22 (m, 0.5H), 4.36-4.21 (m, 2H), 3.70-3.62 (m, 1H), 3.54-3.34 (m, 5H), 3.27- 3.15 (m, 4H), 3.07-2.98 (m, 1H), 2.73-2.54 (m, 2H), 2.39-2.13 (m, 3H), 2.06-1.87 (m, 3H). |
| 221 | 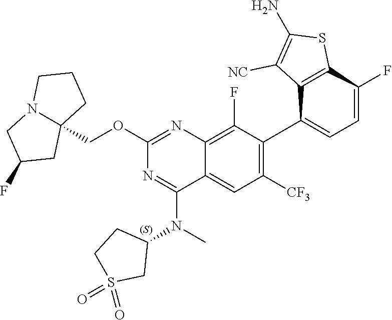 | LCMS m/z [M + 1]: 711.0 1H NMR (400 MHz, CD3OD) δ 8.31 (s, 1H), 7.24- 7.18 (m, 1H), 7.05-6.97 (m, 1H), 5.53-5.43 (m, 1H), 5.39-5.35 (m, 0.5H), 5.26-5.22 (m, 0.5H), 4.35-4.21 (m, 2H), 3.71-3.63 (m, 1H), 3.53-3.33 (m, 5H), 3.26- 3.14 (m, 4H), 3.06-2.98 (m, 1H), 2.71-2.55 (m, 2H), 2.38-2.12 (m, 3H), 2.04-1.86 (m, 3H). |
| 221a | 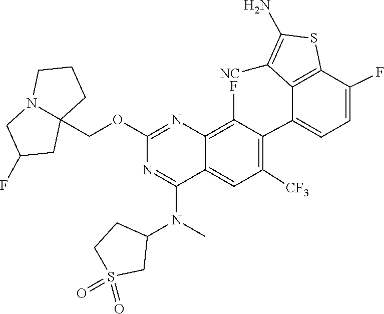 [SMILES: CN(c1nc(OCC23CCCN2CC(F)C3)nc2c(F)c(-c3ccc(F)c4sc(N)c(C#N)c34)c(C(F)(F)F)cc12)C1CCS(=O)(=O)C1] | |
| 222 | 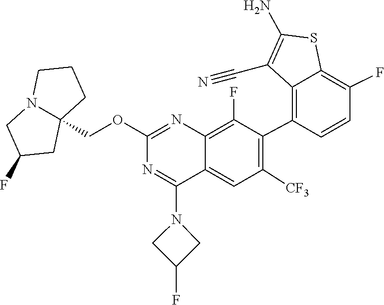 [SMILES: N#Cc1c(N)sc2c(F)ccc(-c3c(C(F)(F)F)cc4c(N5CC(F)C5)nc(OC[C@@]56CCCN5C[C@H](F)C6)nc4c3F)c12] | LCMS m/z [M + 1]: 637.0 1H NMR (400 MHz, CD3OD) δ 8.03 (s, 1H), 7.19 (t, 1H), 7.00 (t, 1H), 5.63 (s, 0.5H), 5.49 (s, 0.5H), 5.37 (s, 0.5H), 5.24 (s, 0.5H), 5.05-4.92 (m, 2H), 4.80- 4.65 (m, 2H), 4.33-4.22 (m, 2H), 3.28-3.17 (m, 3H), 3.03-3.00 (m, 1H), 2.38-2.12 (m, 3H), 2.01-1.87 (m, 3H). |
| 222a | 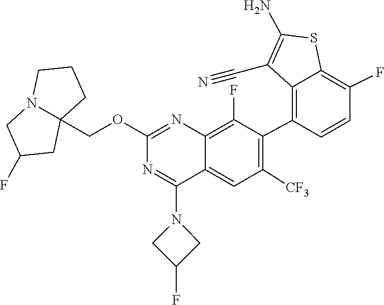 [SMILES: N#Cc1c(N)sc2c(F)ccc(-c3c(C(F)(F)F)cc4c(N5CC(F)C5)nc(OCC56CCCN5CC(F)C6)nc4c3F)c12] | |
| 223 | 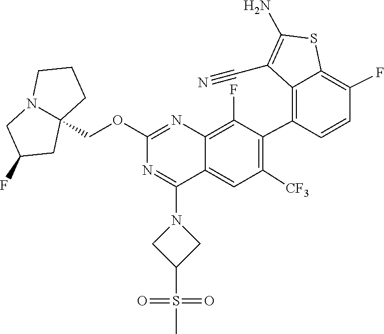 [SMILES: CS(=O)(=O)C1CN(c2nc(OC[C@@]34CCCN3C[C@H](F)C4)nc3c(F)c(-c4ccc(F)c5sc(N)c(C#N)c45)c(C(F)(F)F)cc23)C1] | LCMS m/z [M + 1]: 697.0 1H NMR (400 MHz, CD3OD) δ 8.03 (s, 1H), 7.20 (t, 1H), 7.00 (t, 1H), 5.37 (s, 0.5H), 5.23 (s, 0.5H), 4.56- 4.52 (m, 1H), 4.33-4.22 (m, 2H), 3.30-3.15 (m, 4H), 3.09 (s, 3H), 3.02-2.99 (m, 1H), 2.37-2.12 (m, 3H), 2.01-1.87 (m, 3H). |
| 223a | 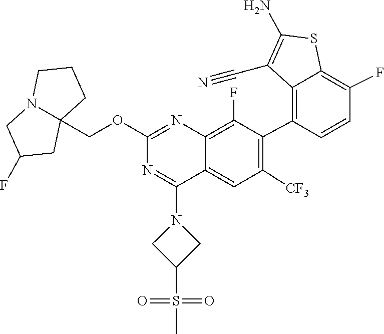 [SMILES: CS(=O)(=O)C1CN(c2nc(OCC34CCCN3CC(F)C4)nc3c(F)c(-c4ccc(F)c5sc(N)c(C#N)c45)c(C(F)(F)F)cc23)C1] | |
| 224 | 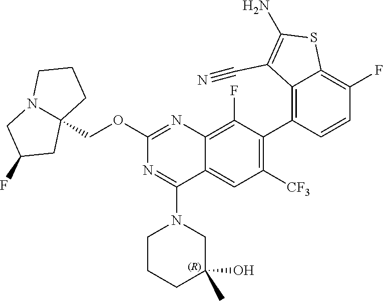 | LCMS m/z [M + 1]: 677.0 1H NMR (400 MHz, CD3OD) δ 8.36 (s, 1H), 7.17- 7.23 (m, 1H), 7.00 (t, 1H), 5.36 (s, 0.5H), 5.23 (s, 0.5H), 4.30-4.33 (d, 2H), 4.23 (d, 1H), 4.10 (d, 1H), 3.53 (d, 1H), 3.39 (m, 1H), 3.16-3.27 (m, 3H), 2.98- 3.04 (m, 1H), 2.12-2.38 (m, 4H), 1.74-2.03 (m, 6H), 1.26 (d, 3H). |
| 224a | 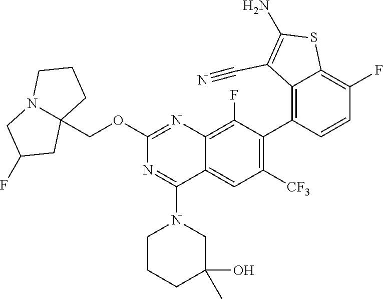 [SMILES: CC1(O)CCCN(c2nc(OCC34CCCN3CC(F)C4)nc3c(F)c(-c4ccc(F)c5sc(N)c(C#N)c45)c(C(F)(F)F)cc23)C1] | |
| 226 | 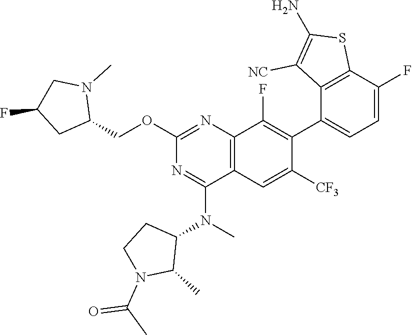 [SMILES: CC(=O)N1CC[C@H](N(C)c2nc(OC[C@@H]3C[C@@H](F)CN3C)nc3c(F)c(-c4ccc(F)c5sc(N)c(C#N)c45)c(C(F)(F)F)cc23)[C@@H]1C] | LCMS m/z [M + 1]: 692.3 1H NMR (400 MHz, CD3OD) δ 8.40 (s, 1H), 7.20- 7.26 (m, 1H), 7.00-7.06 (m, 1H), 5.47 (d, J = 52.4 Hz, 1H), 4.92-5.03 (m, 2H), 4.66-4.77 (m, 2H), 4.23-4.29 (m, 1H), 3.97-4.09 (m, 1H), 3.80-3.86 (m, 1H), 3.57-3.72 (m, 5H), 3.14-3.21 (m, 3H), 2.63-2.76 (m, 2H), 2.38-2.49 (m, 2H), 2.03-2.14 (m, 3H), 1.07-1.19 (m, 3H). |
| 226a | 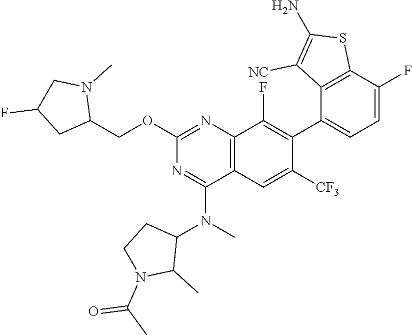 [SMILES: CC(=O)N1CCC(N(C)c2nc(OCC3CC(F)CN3C)nc3c(F)c(-c4ccc(F)c5sc(N)c(C#N)c45)c(C(F)(F)F)cc23)C1C] | |
| 227 | 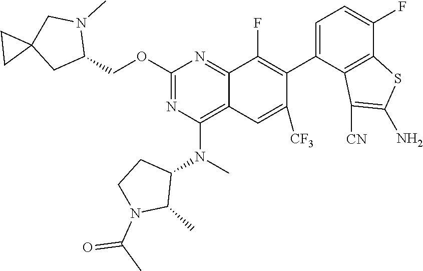 [SMILES: CC(=O)N1CC[C@H](N(C)c2nc(OC[C@@H]3CC4(CC4)CN3C)nc3c(F)c(-c4ccc(F)c5sc(N)c(C#N)c45)c(C(F)(F)F)cc23)[C@@H]1C] | LCMS m/z [M + 1]: 700.0 1H NMR (400 MHz, CD3OD) δ 8.41 (s, 1H), 7.22- 7.25 (m, 1H), 7.06 (t, J = 9.6 Hz, 1H), 4.91-4.98 (m, 1H), 4.63-4.78 (m, 1H), 4.05-4.10 (m, 1H), 3.80-3.85 (m, 1H), 3.68 (s, 3H), 3.56-3.62 (m, 1H), 3.36-3.53 (m, 2H), 3.13 (s, 3H), 2.58-2.73 (m, 1H), 2.38-2.49 (m, 2H), 2.11(d, J = 19.4 Hz, 3H), 1.90-1.98 (m, 1H), 1.29.(d, J = 9.6 Hz, 1H), 1.15-1.18 (m, 1H), 1.06-110 (m, 2H), 0.72-0.92 (m, 5H). |
| 227a | 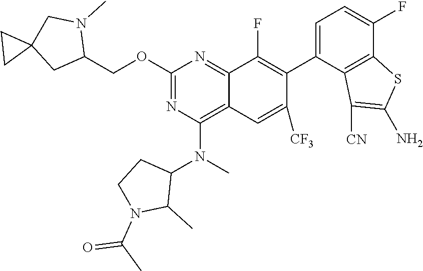 [SMILES: CC(=O)N1CCC(N(C)c2nc(OCC3CC4(CC4)CN3C)nc3c(F)c(-c4ccc(F)c5sc(N)c(C#N)c45)c(C(F)(F)F)cc23)C1C] | |
| 228 | 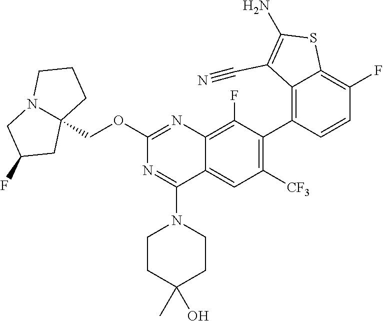 [SMILES: CC1(O)CCN(c2nc(OC[C@@]34CCCN3C[C@H](F)C4)nc3c(F)c(-c4ccc(F)c5sc(N)c(C#N)c45)c(C(F)(F)F)cc23)CC1] | LCMS m/z [M + 1]: 677.0 1H NMR (400 MHz, CD3OD) δ 8.11 (s, 1H), 7.19- 7.22 (m, 1H), 6.98-7.02 (m, 1H), 5.38 (s, 0.5H), 5.24 (s, 0.5H), 4.31-4.35 (m, 1H), 4.23-4.26 (m, 3H), 3.75-3.80 (m, 2H), 3.20-3.26 (m, 3H), 3.01-3.04 (m, 1H), 2.14-2.29 (m, 3H), 1.97-2.03 (m, 2H), 1.75- 1.90 (m, 5H), 1.32 (s, 3H). |
| 228a | 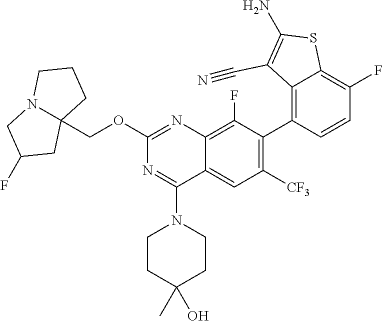 [SMILES: CC1(O)CCN(c2nc(OCC34CCCN3CC(F)C4)nc3c(F)c(-c4ccc(F)c5sc(N)c(C#N)c45)c(C(F)(F)F)cc23)CC1] | |
| 229 | 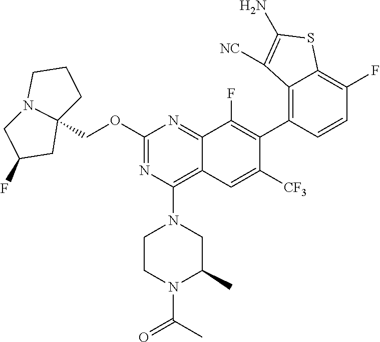 [SMILES: CC(=O)N1CCN(c2nc(OC[C@@]34CCCN3C[C@H](F)C4)nc3c(F)c(-c4ccc(F)c5sc(N)c(C#N)c45)c(C(F)(F)F)cc23)C[C@H]1C] | LCMS m/z [M + 1]: 704.3 1H NMR (400 MHz, CD3OD) δ 8.20 (d, 1H), 7.25- 7.18 (m, 1H), 7.01 (t, 1H,), 5.38 (d, 0.5H), 5.24 (d, 0.5H), 4.82-4.74 (m, 1H), 4.48-4.18 (m, 5H), 3.96- 3.65 (m, 3H), 3.28-3.18 (m, 3H), 3.07-3.00 (m, 1H), 2.40-2.11 (m, 6H), 2.03-1.87 (m, 3H), 1.44-1.29 (m, 3H). |
| 229a | 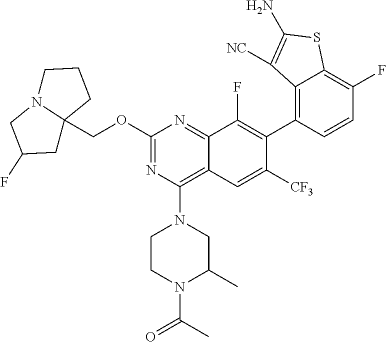 [SMILES: CC(=O)N1CCN(c2nc(OCC34CCCN3CC(F)C4)nc3c(F)c(-c4ccc(F)c5sc(N)c(C#N)c45)c(C(F)(F)F)cc23)CC1C] | |
| 230 | 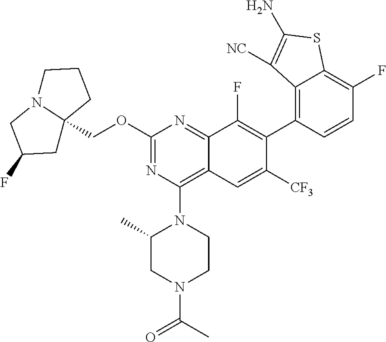 [SMILES: CC(=O)N1CCN(c2nc(OC[C@@]34CCCN3C[C@H](F)C4)nc3c(F)c(-c4ccc(F)c5sc(N)c(C#N)c45)c(C(F)(F)F)cc23)[C@@H](C)C1] | LCMS m/z [M + 1]: 704.3 1H NMR (400 MHz, CD3OD) δ 8.11 (s, 1H), 7.20 (t, 1H), 7.01 (t, 1H), 5.37 (d, 0.5H), 5.23 (d, 0.5H), 4.47-4.22 (m, 4H), 4.04-3.50 (m, 4H), 3.27-3.13 (m, 4H), 3.05-2.98 (m, 1H), 2.39-2.10 (m, 6H), 2.04-1.86 (m, 3H), 1.56-1.43 (m, 3H). |
| 230a | 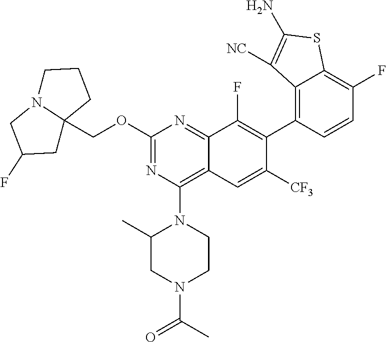 [SMILES: CC(=O)N1CCN(c2nc(OCC34CCCN3CC(F)C4)nc3c(F)c(-c4ccc(F)c5sc(N)c(C#N)c45)c(C(F)(F)F)cc23)C(C)C1] | |
| 231 | 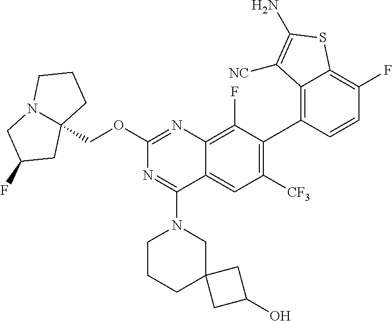 | LCMS m/z [M + 1]: 703.1 1H NMR (400 MHz, CD3OD) δ 8.17 (s, 1H), 7.23- 7.21 (m, 1H), 7.04-7.00 (m, 1H), 5.60 (s, 0.5H), 5.47 (s, 0.5H), 4.69-4.59 (m, 2H), 4.28-4.24 (m, 1H), 4.00-3.86 (m, 6H), 3.41 (s, 1H), 3.13 (s, 1H), 2.59- 2.16 (m, 8H), 1.81-1.68 (m, 6H). |
| 231a | 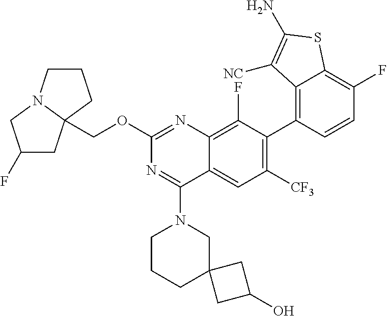 [SMILES: N#Cc1c(N)sc2c(F)ccc(-c3c(C(F)(F)F)cc4c(N5CCCC6(CC(O)C6)C5)nc(OCC56CCCN5CC(F)C6)nc4c3F)c12] | |
| 232 | 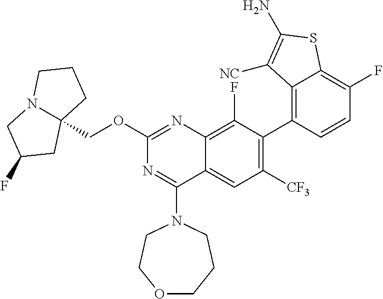 [SMILES: N#Cc1c(N)sc2c(F)ccc(-c3c(C(F)(F)F)cc4c(N5CCCOCC5)nc(OC[C@@]56CCCN5C[C@H](F)C6)nc4c3F)c12] | LCMS m/z [M + 1]: 663.19 1H NMR (400 MHz, CD3OD) δ 8.26 (s, 1H), 7.23- 7.17 (m, 1H), 7.04-6.97 (m, 1H), 5.37 (s, 0.5H), 5.24 (s, 0.5H), 4.34-4.27 (m, 1H), 4.24-4.15 (m, 4H), 4.07-3.96 (m, 2H), 3.89-3.78 (m, 2H), 3.28-3.17 (m, 4H), 3.05-2.98 (m, 1H), 2.39-2.10 (m, 5H), 2.04-1.84 (m, 3H). |
| 232a | 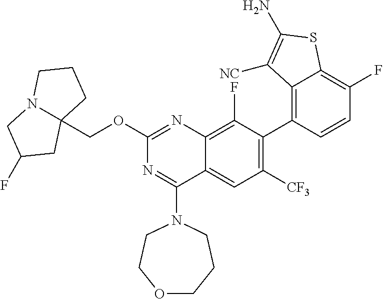 [SMILES: N#Cc1c(N)sc2c(F)ccc(-c3c(C(F)(F)F)cc4c(N5CCCOCC5)nc(OCC56CCCN5CC(F)C6)nc4c3F)c12] | |
| 233 | 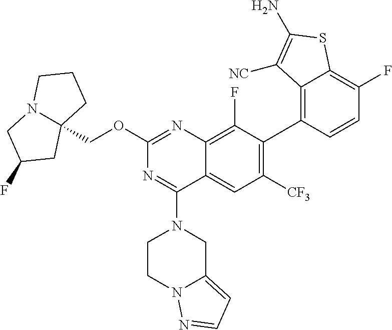 | LCMS m/z [M + 1]: 685.19 1H NMR (400 MHz, CD3OD) δ 8.22 (s, 1H), 7.54- 7.47 (d, 1H), 7.26-7.18 (m, 1H), 7.05-6.98 (m, 1H), 6.27-6.20 (m, 1H), 5.36 (s, 0.5H), 5.23 (s, 2.5H), 4.52-4.22 (m, 6H), 3.27-3.16 (m, 3H), 3.04-2.97 (m, 1H), 2.38-2.11 (m, 3H), 2.03-1.84 (m, 3H). |
| 233a | 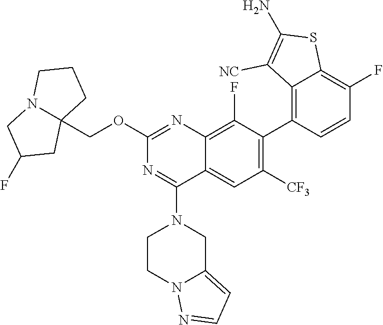 [SMILES: N#Cc1c(N)sc2c(F)ccc(-c3c(C(F)(F)F)cc4c(N5CCn6nccc6C5)nc(OCC56CCCN5CC(F)C6)nc4c3F)c12] | |
| 234 | 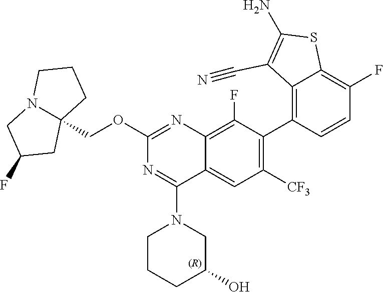 | LCMS m/z [M + 1]: 663.0 1H NMR (400 MHz, CD3OD) δ 8.26-8.28 (d, 1H), 7.18-7.22 (m, 1H), 6.98-7.02 (m, 1H), 5.38 (s, 0.5H), 5.25 (s, 0.5H), 4.32-4.36 (m, 1H), 4.23-4.27 (m, 1H), 4.11-4.17 (m, 1H), 3.86-3.94 (m, 3H), 3.63-3.70 (m, 1H), 3.21-3.25 (m, 3H), 3.00-3.07 (m, 1H), 2.14-2.31 (m, 3H), 1.97-2.06 (m, 4H), 1.88-1.94 (m, 1H), 1.71- 1.73 (m, 2H). |
| 234a | 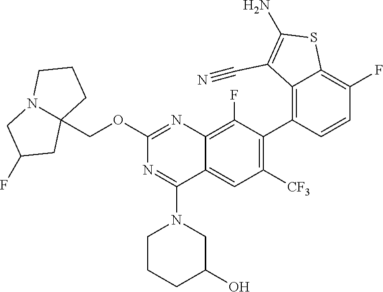 [SMILES: N#Cc1c(N)sc2c(F)ccc(-c3c(C(F)(F)F)cc4c(N5CCCC(O)C5)nc(OCC56CCCN5CC(F)C6)nc4c3F)c12] | |
| 235 | 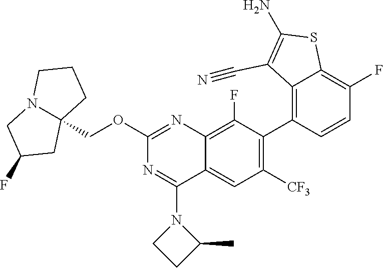 [SMILES: C[C@H]1CCN1c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 633.0 1H NMR (400 MHz, CD3OD) δ 8.06 (s, 1H), 7.17- 7.20 (m, 1H), 6.97-7.02 (m, 1H), 5.39 (s, 0.5H), 5.26 (s, 0.5H), 4.99 (s, 1H), 4.62 (s, 1H), 4.25-4.32 (m, 2H), 3.39-3.42 (m, 1H), 3.16-3.24 (m, 2H), 3.03-3.07 (m, 1H), 2.75-2.79 (m, 1H), 2.08-2.48 (m, 5H), 1.98- 2.04 (m, 2H), 1.88-1.91 (m, 1H), 1.66-1.69 (m, 3H). |
| 235a | 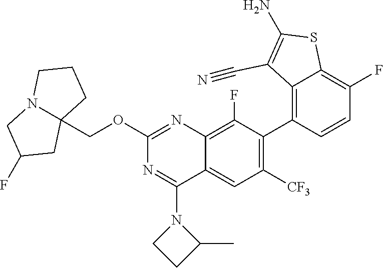 [SMILES: CC1CCN1c1nc(OCC23CCCN2CC(F)C3)nc2c(F)c(-c3ccc(F)c4sc(N)c(C#N)c34)c(C(F)(F)F)cc12] | |
| 236 | 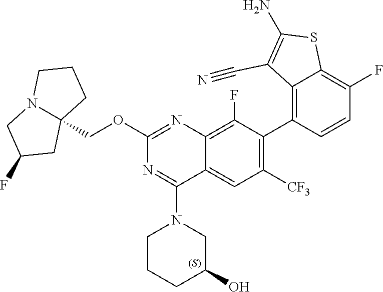 | LCMS m/z [M + 1]: 663.2 1H NMR (400 MHz, CD3OD) δ 8.27 (d, 1H), 7.20 (t, 1H), 7.02 (t, 1H), 5.38 (s, 0.5H), 5.24 (s, 0.5H), 4.31-4.35 (m, 1H), 4.24-4.29 (m, 1H), 4.13-4.16 (m, 1H), 3.85-3.94 (m, 3H), 3.63-3.70 (m, 1H), 3.19-3.29 (m, 3H), 3.00-3.07 (m, 1H), 2.14-2.35 (m, 3H), 1.88- 2.06 (m, 5H), 1.69-1.75 (m, 2H). |
| 237 | 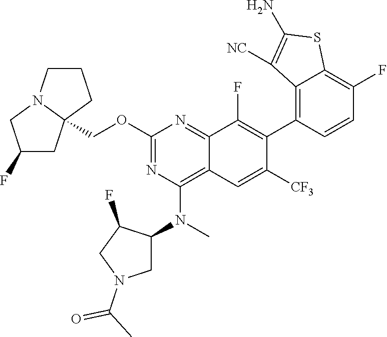 [SMILES: CC(=O)N1C[C@@H](F)[C@@H](N(C)c2nc(OC[C@@]34CCCN3C[C@H](F)C4)nc3c(F)c(-c4ccc(F)c5sc(N)c(C#N)c45)c(C(F)(F)F)cc23)C1] | LCMS m/z [M + 1]: 722.1 1H NMR (400 MHz, CD3OD) δ 8.35 (d, 1H), 7.17- 7.27 (m, 1H), 7.01 (t, 1H), 5.80 (d, 0.5H), 5.66 (d, 0.5H), 5.35 (s, 0.5H), 5.11-5.29 (m, 1.5H), 4.14-4.32 (m, 3H), 3.73-4.07 (m, 3H), 3.63 (d, 3H), 3.16-3.29 (m, 3H), 2.96-3.06 (m, 1H), 1.81-2.04 (m, 3H). |
| 237a | 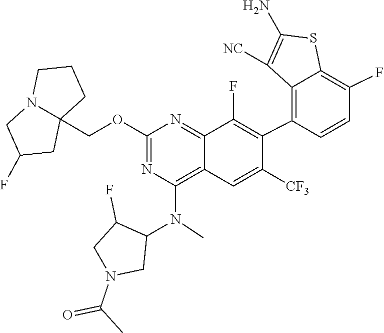 [SMILES: CC(=O)N1CC(F)C(N(C)c2nc(OCC34CCCN3CC(F)C4)nc3c(F)c(-c4ccc(F)c5sc(N)c(C#N)c45)c(C(F)(F)F)cc23)C1] | |
| 238 | 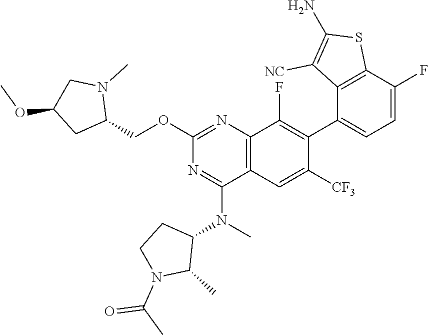 [SMILES: CO[C@@H]1C[C@@H](COc2nc(N(C)[C@H]3CCN(C(C)=O)[C@H]3C)c3cc(C(F)(F)F)c(-c4ccc(F)c5sc(N)c(C#N)c45)c(F)c3n2)N(C)C1] | LCMS m/z [M + 1]: 704.2 1H NMR (400 MHz, CD3OD) δ 8.34 (s, 1H), 7.19- 7.25 (m, 1H), 6.99-7.04 (m, 1H), 4.89-4.99 (m, 1H), 4.43-4.61 (m, 4H), 3.95-4.02 (m, 1H), 3.77- 3.85 (m, 1H), 3.39-3.71 (m, 6H), 3.26-3.28 (m, 1H), 3.02-3.10 (m, 1H), 2.60-2.71 (m, 1H), 2.50- 2.56 (m, 3H), 2.39-2.48 (m, 2H), 1.91-2.20 (m, 7H). |
| 238a | 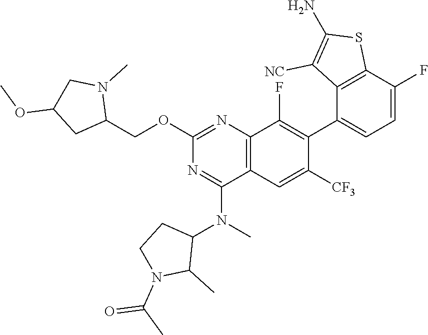 [SMILES: COC1CC(COc2nc(N(C)C3CCN(C(C)=O)C3C)c3cc(C(F)(F)F)c(-c4ccc(F)c5sc(N)c(C#N)c45)c(F)c3n2)N(C)C1] | |
| 239 | 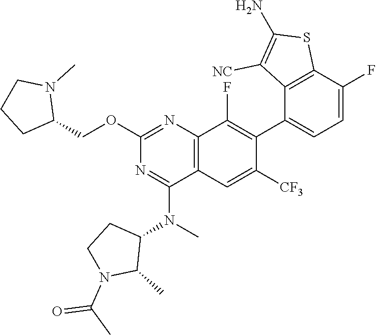 [SMILES: CC(=O)N1CC[C@H](N(C)c2nc(OC[C@@H]3CCCN3C)nc3c(F)c(-c4ccc(F)c5sc(N)c(C#N)c45)c(C(F)(F)F)cc23)[C@@H]1C] | LCMS m/z [M + 1]: 674.3 1H NMR (400 MHz, CD3OD) δ 8.35 (s, 1H), 7.19- 7.26 (m, 1H), 7.01 (t, J = 8.9 Hz, 1H), 4.91-5.00 (m, 1H), 4.41-4.62 (m, 3H), 3.76-3.86 (m, 1H), 3.46-3.70 (m, 5H), 3.13-3.20 (m, 1H), 2.91-3.00 (m, 1H), 2.42-2.69 (m, 6H), 2.00-2.22 (m, 5H), 1.72-1.91 (m, 4H). |
| 239a | 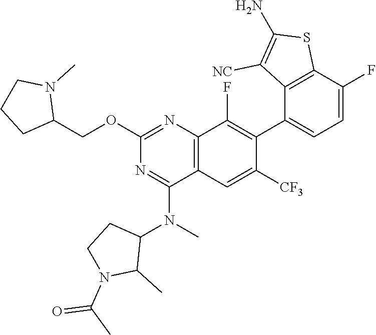 [SMILES: CC(=O)N1CCC(N(C)c2nc(OCC3CCCN3C)nc3c(F)c(-c4ccc(F)c5sc(N)c(C#N)c45)c(C(F)(F)F)cc23)C1C] | |
| 240 | 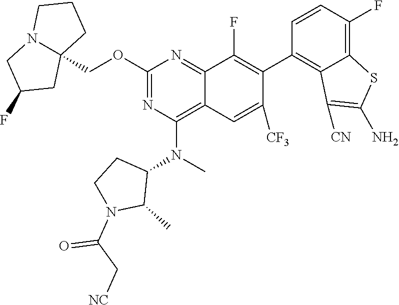 | LCMS m/z [M + 1]: 734.2 1H NMR (400 MHz, CD3OD) δ 8.42 (s, 1H), 7.21- 7.25 (m, 1H), 7.06 (t, J = 8.8 Hz, 1H), 5.57 (d, J = 52.4 Hz, 1H), 5.01-5.05 (m, 1H), 4.65-4.82 (m, 1H), 4.24-4.28 (m, 2H), 3.84-4.16 (m, 3H), 3.72-3.77 (m, 1H), 3.62 (s, 3H), 3.43-3.59 (m, 2H), 2.33-2.74 (m, 7H), 2.17-2.36 (m, 2H), 1.26-1.34 (m, 2H), 1.12-1.15 (m, 3H). |
| 240a | 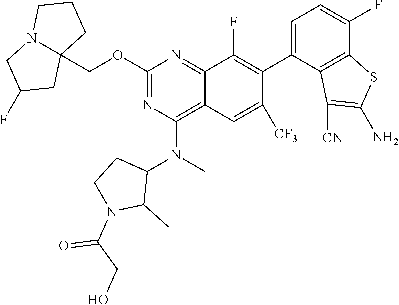 [SMILES: CC1C(N(C)c2nc(OCC34CCCN3CC(F)C4)nc3c(F)c(-c4ccc(F)c5sc(N)c(C#N)c45)c(C(F)(F)F)cc23)CCN1C(=O)CO] | |
| 241 | 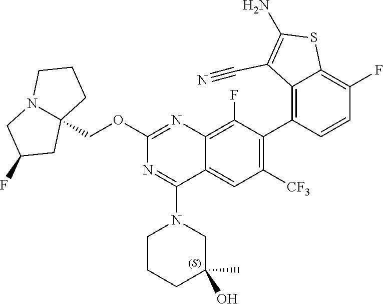 | LCMS m/z [M + 1]: 677.2 1H NMR (400 MHz, CD3OD) δ 8.40 (d, 1H), 7.19- 7.24 (m, 1H), 6.99-7.04 (m, 1H), 5.39 (s, 0.5H), 5.26 (s, 0.5H), 4.25-4.36 (m, 3H), 4.12 (d, 1H), 3.52-3.56 (m, 1H), 3.35-3.44 (m, 2H), 3.21-3.26 (m, 2H), 3.02- 3.08 (m, 1H), 2.24-2.40 (m, 2H), 2.13-2.20 (m, 2H), 1.76-2.04 (m, 6H), 1.27 (d, 3H). |
| 241a | 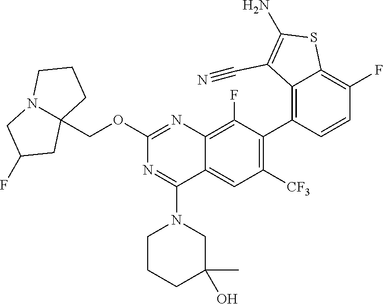 [SMILES: CC1(O)CCCN(c2nc(OCC34CCCN3CC(F)C4)nc3c(F)c(-c4ccc(F)c5sc(N)c(C#N)c45)c(C(F)(F)F)cc23)C1] | |
| 242 | 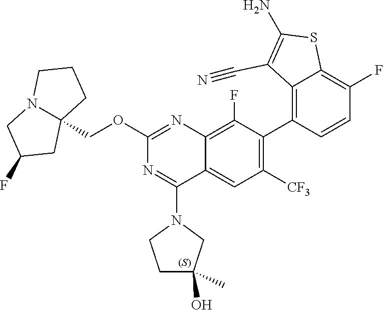 | LCMS m/z [M + 1]: 663.0 1H NMR (400 MHz, CD3OD) δ 8.436 (s, 1H), 7.25- 7.20 (m, 1H), 7.027 (t, 1H), 5.409 (s, 0.5H), 5.274 (s, 0.5H), 4.42-4.23 (m, 3H), 4.20-4.12 (m, 1H), 4.05- 3.91 (m, 2H), 3.45-3.34 (m, 1H), 3.30-3.20 (m, 2H), 3.10-3.03 (m, 1H), 2.47-2.34 (m, 1H), 2.31-2.25 (m, 1H), 2.23-2.09 (m, 3H), 2.07-1.99 (m, 2H), 1.96-1.89 (m, 1H), 1.547 (s, 3H). |
| 242a | 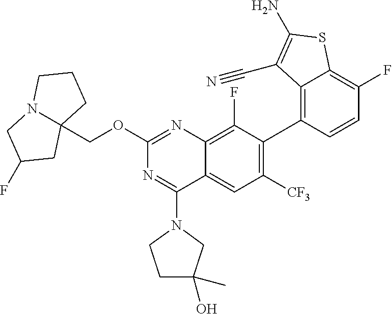 [SMILES: CC1(O)CCN(c2nc(OCC34CCCN3CC(F)C4)nc3c(F)c(-c4ccc(F)c5sc(N)c(C#N)c45)c(C(F)(F)F)cc23)C1] | |
| 243 | 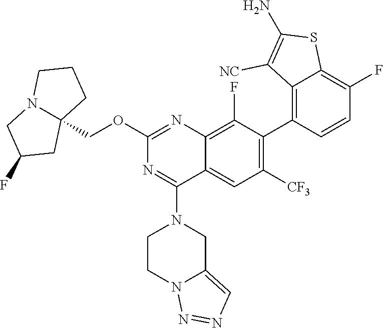 [SMILES: N#Cc1c(N)sc2c(F)ccc(-c3c(C(F)(F)F)cc4c(N5CCn6nncc6C5)nc(OC[C@@]56CCCN5C[C@H](F)C6)nc4c3F)c12] | LCMS m/z [M + 1]: 686.2 1H NMR (400 MHz, CD3OD) δ 8.27 (s, 1H), 7.90 (s, 1H), 7.27-7.23 (m, 1H), 7.07-7.00 (m, 1H), 5.40 (s, 0.58H), 5.35 (s, 2H), 5.27 (s, 0.52H), 4.80-4.77 (m, 2H), 4.51-4.30 (m, 4H), 3.38-3.33 (m, 1H), 3.31- 3.24 (m, 2H), 3.07-3.03 (m, 1H), 2.42-2.17 (m, 3H), 2.05-1.92 (m, 3H). |
| 243a | 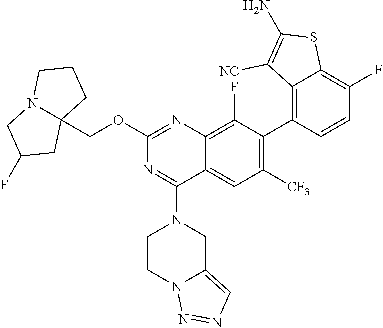 [SMILES: N#Cc1c(N)sc2c(F)ccc(-c3c(C(F)(F)F)cc4c(N5CCn6nncc6C5)nc(OCC56CCCN5CC(F)C6)nc4c3F)c12] | |
| 244 | 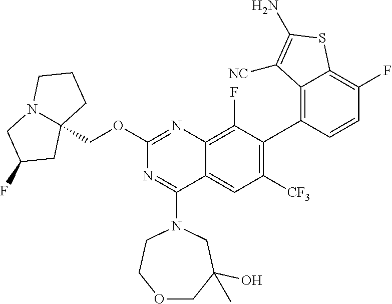 [SMILES: CC1(O)COCCN(c2nc(OC[C@@]34CCCN3C[C@H](F)C4)nc3c(F)c(-c4ccc(F)c5sc(N)c(C#N)c45)c(C(F)(F)F)cc23)C1] | LCMS m/z [M + 1]: 693.0 1H NMR (400 MHz, CD3OD) δ 8.94 (s, 1H), 7.25- 7.22 (m, 1H), 7.06-7.01 (m, 1H), 5.55 (s, 0.5H), 5.41 (s, 0.5H), 4.62-4.44 (m, 5H), 4.16-3.68 (m, 9H), 2.47-2.05 (m, 6H), 1.28 (s, 3H). |
| 244a | 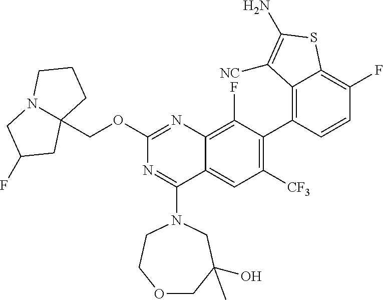 [SMILES: CC1(O)COCCN(c2nc(OCC34CCCN3CC(F)C4)nc3c(F)c(-c4ccc(F)c5sc(N)c(C#N)c45)c(C(F)(F)F)cc23)C1] | |
| 245 | 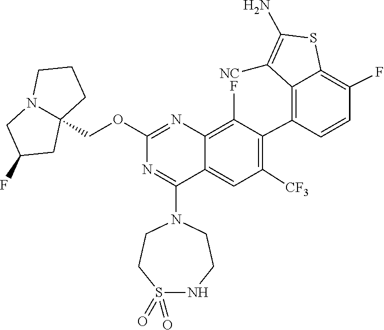 [SMILES: N#Cc1c(N)sc2c(F)ccc(-c3c(C(F)(F)F)cc4c(N5CCNS(=O)(=O)CC5)nc(OC[C@@]56CCCN5C[C@H](F)C6)nc4c3F)c12] | LCMS m/z [M + 1]: 712.0 1H NMR (400 MHz, CD3OD) δ 8.30 (s, 1H), 7.24 (t, 1H), 7.03 (t, 1H), 5.44 (s, 0.5H), 5.31 (s, 0.5H), 4.46- 4.31 (m, 5H), 3.74-3.36 (m, 8H), 3.18-2.98 (m, 1H), 2.46-2.22 (m, 3H), 2.10-1.95 (m, 3H). |
| 245a | 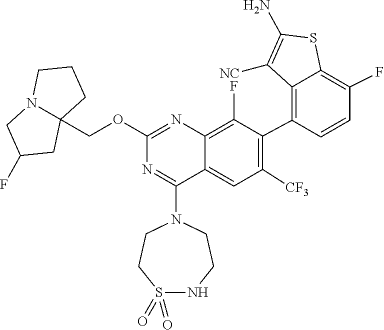 [SMILES: N#Cc1c(N)sc2c(F)ccc(-c3c(C(F)(F)F)cc4c(N5CCNS(=O)(=O)CC5)nc(OCC56CCCN5CC(F)C6)nc4c3F)c12] | |
| 246 | 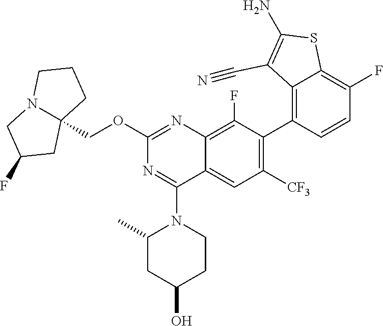 [SMILES: C[C@H]1C[C@H](O)CCN1c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 677.1 1H NMR (400 MHz, CD3OD) δ 8.08 (s, 1H), 7.22 (t, 1H), 7.02 (t, 1H), 5.39 (s, 0.5H), 5.26 (s, 0.5H), 4.96 (s, 1H), 4.44 (d, 1H), 4.23-4.37 (m, 2H), 4.15 (m, 1H), 3.55-3.61 (m, 1H), 3.19-3.29 (m, 3H), 3.02-3.06 (m, 1H), 2.24-2.41 (m, 2H), 1.90-2.20 (m, 6H), 1.68- 1.75 (m, 2H), 1.55 (t, 3H). |
| 246a | 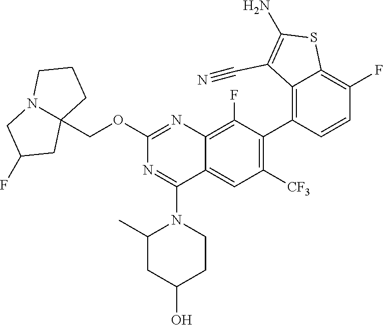 [SMILES: CC1CC(O)CCN1c1nc(OCC23CCCN2CC(F)C3)nc2c(F)c(-c3ccc(F)c4sc(N)c(C#N)c34)c(C(F)(F)F)cc12] | |
| 247 | 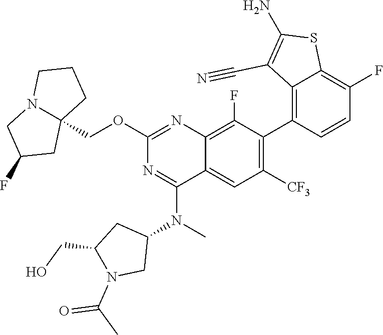 [SMILES: CC(=O)N1C[C@@H](N(C)c2nc(OC[C@@]34CCCN3C[C@H](F)C4)nc3c(F)c(-c4ccc(F)c5sc(N)c(C#N)c45)c(C(F)(F)F)cc23)C[C@H]1CO] | LCMS m/z [M + 1]: 734.1 1H NMR (400 MHz, CD3OD) δ 8.40 (s, 1H), 7.21- 7.29 (m, 1H), 7.04 (t, 1H), 5.64 (s, 0.5H), 5.51 (s, 0.5H), 5.20-5.38 (m, 1H), 4.61-4.80 (m, 2H), 4.19- 4.30 (m, 1H), 3.82-4.15 (m, 5H), 3.62-3.79 (m, 2H), 3.42-3.61 (m, 4H), 2.10-2.78 (m, 1H). |
| 247a | 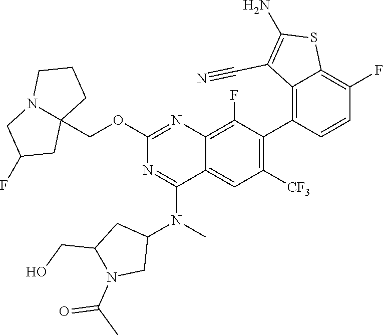 [SMILES: CC(=O)N1CC(N(C)c2nc(OCC34CCCN3CC(F)C4)nc3c(F)c(-c4ccc(F)c5sc(N)c(C#N)c45)c(C(F)(F)F)cc23)CC1CO] | |
| 248 | 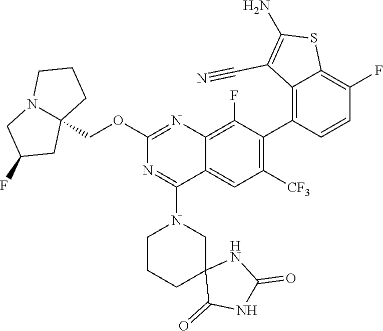 [SMILES: N#Cc1c(N)sc2c(F)ccc(-c3c(C(F)(F)F)cc4c(N5CCCC6(C5)NC(=O)NC6=O)nc(OC[C@@]56CCCN5C[C@H](F)C6)nc4c3F)c12] | LCMS m/z [M + 1]: 732.2 1H NMR (400 MHz, CD3OD) δ 8.29 (s, 1H), 7.20- 7.28 (m, 1H), 7.03 (t, 1H), 5.42 (s, 0.5H), 5.29 (s, 0.5H), 4.25-4.56 (m, 4H), 3.33-3.70 (m, 5H), 3.04- 3.14 (m, 1H), 1.86-2.49 (m, 11H). |
| 248a | 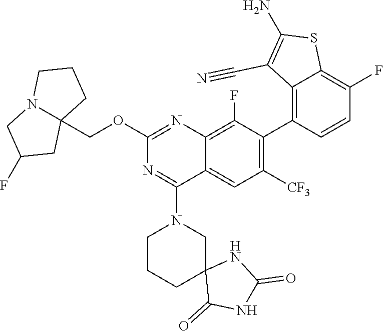 [SMILES: N#Cc1c(N)sc2c(F)ccc(-c3c(C(F)(F)F)cc4c(N5CCCC6(C5)NC(=O)NC6=O)nc(OCC56CCCN5CC(F)C6)nc4c3F)c12] | |
| 249 | 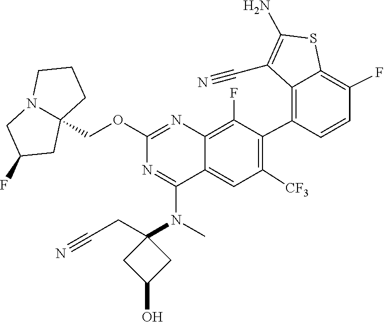 | LCMS m/z [M + 1]: 702.2 1HNMR (400 MHz, CD3OD) δ 8.36 (s, 1H), 7.20- 7.26 (m, 1H), 7.02 (t, 1H), 5.39 (s, 0.5H), 5.26 (s, 0.5H), 4.22-4.37 (m, 3H), 3.56 (s, 3H), 3.37 (s, 2H), 3.22-3.30 (m, 2H), 3.01-3.09 (m, 2H), 2.20-2.56 (m, 5H), 1.88-2.20 (m, 5H). |
| 249a | 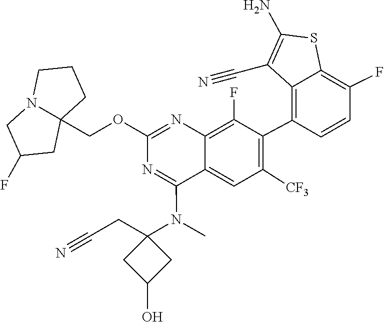 [SMILES: CN(c1nc(OCC23CCCN2CC(F)C3)nc2c(F)c(-c3ccc(F)c4sc(N)c(C#N)c34)c(C(F)(F)F)cc12)C1(CC#N)CC(O)C1] | |
| 251 | 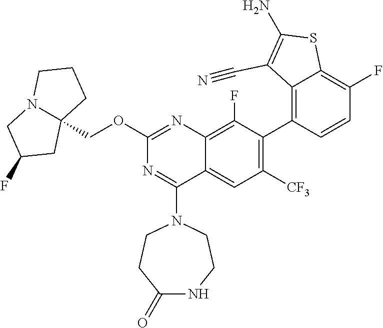 [SMILES: N#Cc1c(N)sc2c(F)ccc(-c3c(C(F)(F)F)cc4c(N5CCNC(=O)CC5)nc(OC[C@@]56CCCN5C[C@H](F)C6)nc4c3F)c12] | LCMS m/z [M + 1]: 676.0 1H NMR (400 MHz, CD3OD) δ 8.13 (s, 1H), 7.19- 7.22 (m, 1H), 7.00 (t, 1H), 5.37 (s, 0.5H), 5.23 (s, 0.5H), 4.25-4.34 (m, 2H), 4.05-4.13 (m, 4H), 3.60 (s, 2H), 3.12-3.25 (m, 3H), 2.99-3.04 (m, 3H), 2.13-2.34 (m, 3H), 1.88-2.03 (m, 3H). |
| 251a | 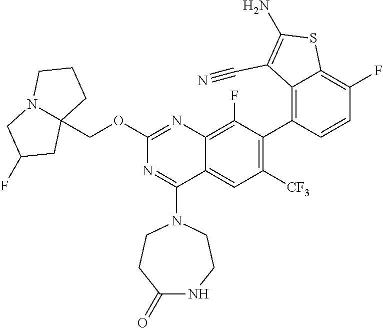 [SMILES: N#Cc1c(N)sc2c(F)ccc(-c3c(C(F)(F)F)cc4c(N5CCNC(=O)CC5)nc(OCC56CCCN5CC(F)C6)nc4c3F)c12] | |
| 252 | 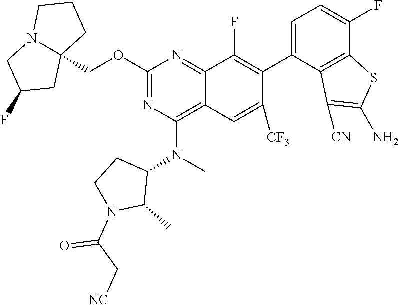 [SMILES: C[C@H]1[C@@H](N(C)c2nc(OC[C@@]34CCCN3C[C@H](F)C4)nc3c(F)c(-c4ccc(F)c5sc(N)c(C#N)c45)c(C(F)(F)F)cc23)CCN1C(=O)CC#N] | LCMS m/z [M + 1]: 743.2 1H NMR (400 MHz, CD3OD) δ 8.41 (s, 1H), 7.22- 7.24 (m, 1H), 7.04 (t, J = 8.8 Hz, 1H), 5.57 (d, J = 51.88 Hz, 1H), 4.99-5.05 (m, 1H), 4.60-4.76 (m, 2H), 3.71-4.05 (m, 6H), 3.41-3.66 (m, 6H), 2.33-2.76 (m, 7H), 2.16-2.21 (m, 1H), 1.07-1.10 (m, 3H). |
| 252a | 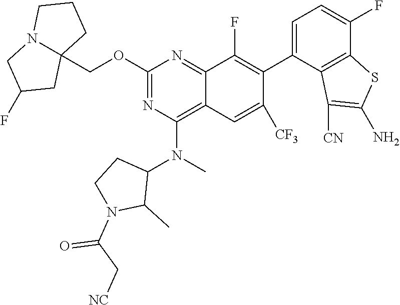 [SMILES: CC1C(N(C)c2nc(OCC34CCCN3CC(F)C4)nc3c(F)c(-c4ccc(F)c5sc(N)c(C#N)c45)c(C(F)(F)F)cc23)CCN1C(=O)CC#N] | |
| 253 | 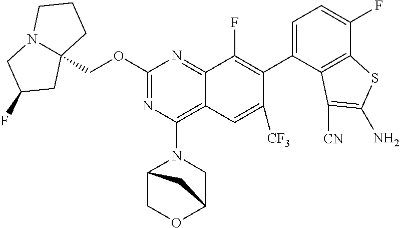 [SMILES: N#Cc1c(N)sc2c(F)ccc(-c3c(C(F)(F)F)cc4c(N5C[C@H]6C[C@@H]5CO6)nc(OC[C@@]56CCCN5C[C@H](F)C6)nc4c3F)c12] | LCMS m/z [M + 1]: 661.2 1H NMR (400 MHz, CD3OD) δ 8.35 (s, 1H), 7.19- 7.24 (m, 1H), 7.02 (t, J = 9.3, Hz, 1H), 5.48-5.61 (m, 2H), 4.56-4.76 (m, 2H), 4.33 (d, J = 9.9 Hz, 1H), 3.80-4.12 (m, 6H), 3.41-3.49 (m, 1H), 2.05-2.79 (m, 9H). |
| 253a | 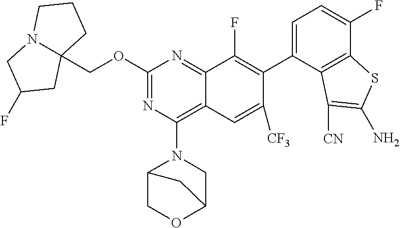 [SMILES: N#Cc1c(N)sc2c(F)ccc(-c3c(C(F)(F)F)cc4c(N5CC6CC5CO6)nc(OCC56CCCN5CC(F)C6)nc4c3F)c12] | |
| 254 | 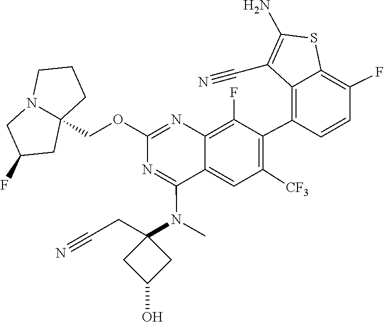 | LCMS m/z [M + 1]: 702.1 1HNMR (400 MHz, CD3OD) δ 8.38 (s, 1H), 7.20- 7.27 (m, 1H), 7.03 (t, 1H), 5.49 (s, 0.5H), 5.36 (s, 0.5H), 4.61 (s, 1H), 4.37-4.45 (m, 2H), 3.82 (s, 3H), 3.45-3.55 (m, 2H), 3.15-3.27 (m, 2H), 2.92-3.02 (m, 2H), 2.35-2.48 (m, 3H), 2.22-2.28 (m, 1H), 2.10-2.18 (m, 1H), 1.97-2.07 (m, 5H). |
| 254a | 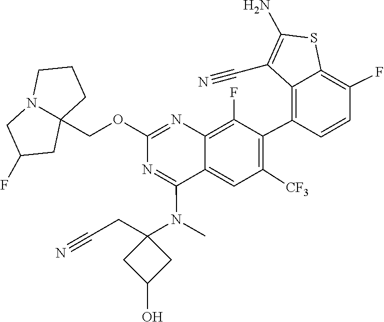 [SMILES: CN(c1nc(OCC23CCCN2CC(F)C3)nc2c(F)c(-c3ccc(F)c4sc(N)c(C#N)c34)c(C(F)(F)F)cc12)C1(CC#N)CC(O)C1] | |
| 255 | 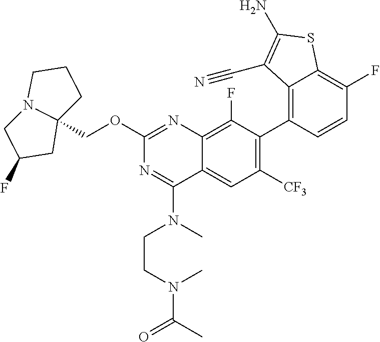 [SMILES: CC(=O)N(C)CCN(C)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 692 1H NMR (400 MHz, CD3OD) δ 8.38-8.34 (m, 1H),7.21-7.18 (m, 1H), 7.00 (t, 1H), 5.38 (s, 0.5H), 5.24 (s, 0.5H), 4.32-4.28 (m, 2H), 4.13-4.07 (m, 2H), 3.87-3.74 (m, 2H), 3.72-3.56 (m, 3H), 3.29-3.19 (m, 4H), 3.13-3.01 (m, 3H), 2.41-2.10 (m, 4H), 2.07-1.78 (m, 5H) |
| 255a | 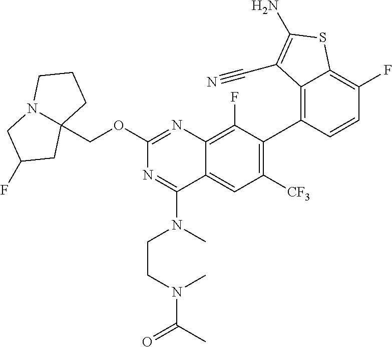 [SMILES: CC(=O)N(C)CCN(C)c1nc(OCC23CCCN2CC(F)C3)nc2c(F)c(-c3ccc(F)c4sc(N)c(C#N)c34)c(C(F)(F)F)cc12] | |
| 256 | 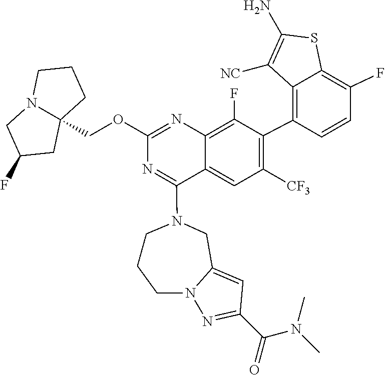 [SMILES: CN(C)C(=O)c1cc2n(n1)CCCN(c1nc(OC[C@@]34CCCN3C[C@H](F)C4)nc3c(F)c(-c4ccc(F)c5sc(N)c(C#N)c45)c(C(F)(F)F)cc13)C2] | LCMS m/z [M + 1]: 770.2 1H NMR (400 MHz, CD3OD) δ 8.22 (s, 1H), 7.17- 7.25 (m, 1H), 7.00 (t, 1H), 6.73 (s, 1H), 5.36 (s, 0.5H), 5.23 (s, 0.5H), 5.13 (s, 2H), 4.53 (t, 2H), 4.15- 4.40 (m, 4H), 3.33 (s, 3H), 3.15-3.27 (m, 3H), 2.94- 3.12 (m, 4H), 2.11-2.45 (m, 5H), 1.84-2.04 (m, 3H). |
| 256a | 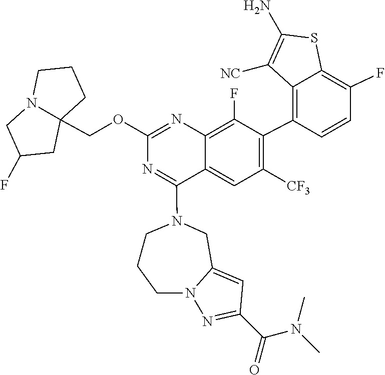 [SMILES: CN(C)C(=O)c1cc2n(n1)CCCN(c1nc(OCC34CCCN3CC(F)C4)nc3c(F)c(-c4ccc(F)c5sc(N)c(C#N)c45)c(C(F)(F)F)cc13)C2] | |
| 257 | 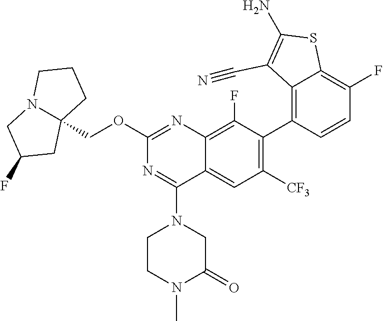 [SMILES: CN1CCN(c2nc(OC[C@@]34CCCN3C[C@H](F)C4)nc3c(F)c(-c4ccc(F)c5sc(N)c(C#N)c45)c(C(F)(F)F)cc23)CC1=O] | LCMS m/z [M + 1]: 676.0 1H NMR (400 MHz, CD3OD) δ 8.20 (s, 1H), 7.20- 7.23 (m, 1H), 6.99-7.03 (m, 1H), 5.41 (s, 0.5H), 5.28 (s, 0.5H), 4.62 (m, 2H), 4.30-4.41 (m, 3H), 4.18-4.23 (m, 1H), 3.61-3.68 (m, 2H), 3.35-3.47 (m, 3H), 3.01- 3.12 (m, 4H), 2.16-2.39 (m, 3H), 1.93-2.07 (m, 3H). |
| 257a | 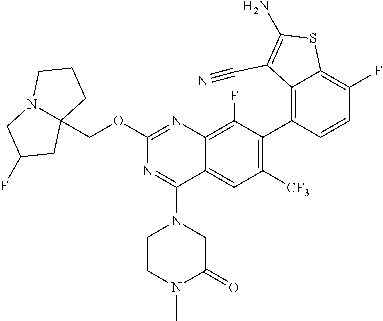 [SMILES: CN1CCN(c2nc(OCC34CCCN3CC(F)C4)nc3c(F)c(-c4ccc(F)c5sc(N)c(C#N)c45)c(C(F)(F)F)cc23)CC1=O] | |
| 258 | 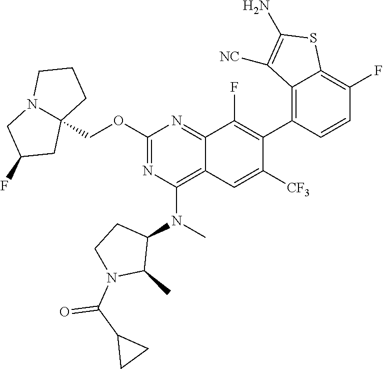 [SMILES: C[C@@H]1[C@H](N(C)c2nc(OC[C@@]34CCCN3C[C@H](F)C4)nc3c(F)c(-c4ccc(F)c5sc(N)c(C#N)c45)c(C(F)(F)F)cc23)CCN1C(=O)C1CC1] | LCMS m/z [M + 1]: 744.3 1H NMR (400 MHz, CD3OD) δ 8.39-8.46 (m, 1H), 7.24 (dd, J = 8.3, 4.9 Hz, 1H), 7.03 (t, J = 8.9 Hz, 1H), 5.55 (d, J = 52.6 Hz, 1H), 4.95-5.10 (m, 2H), 4.58-4.76 (m, 2H), 3.71-4.07 (m, 5H), 3.62-3.71 (m, 3H), 3.44-3.50 (m, 1H), 2.46-2.77 (m, 4H), 2.28-2.45 (m, 3H), 2.07-2.21 (m, 1H), 1.81-1.90 (m, 1H), 0.84-1.26 (m, 7H). |
| 258a | 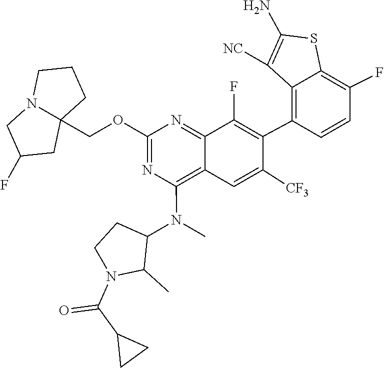 [SMILES: CC1C(N(C)c2nc(OCC34CCCN3CC(F)C4)nc3c(F)c(-c4ccc(F)c5sc(N)c(C#N)c45)c(C(F)(F)F)cc23)CCN1C(=O)C1CC1] | |
| 259 | 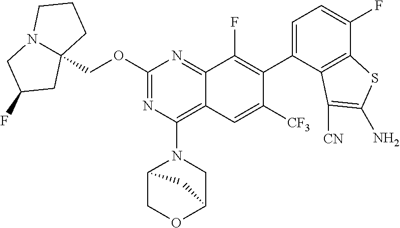 [SMILES: N#Cc1c(N)sc2c(F)ccc(-c3c(C(F)(F)F)cc4c(N5C[C@@H]6C[C@H]5CO6)nc(OC[C@@]56CCCN5C[C@H](F)C6)nc4c3F)c12] | LCMS m/z [M + 1]: 661.3 1H NMR (400 MHz, CD3OD) δ 8.35 (s, 1H), 7.19- 7.26 (m, 1H), 6.99-7.06 (m, 1H), 5.54 (d, J = 51.6 Hz, 1H), 5.53-5.58 (m, 1H), 4.59-4.78 (m, 2H), 4.29-4.37 (m, 1H), 3.97-4.12 (m, 4H), 3.78-3.97 (m, 3H), 3.42-3.52 (m, 1H), 2.57-2.75 (m, 2H), 2.31-2.48 (m, 3H), 2.08-2.20 (m, 3H). |
| 260 | 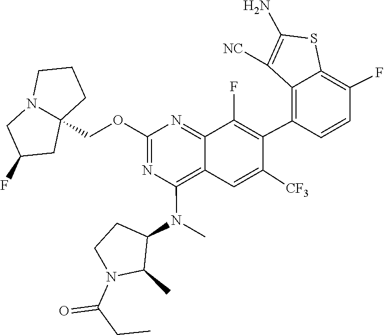 [SMILES: CCC(=O)N1CC[C@@H](N(C)c2nc(OC[C@@]34CCCN3C[C@H](F)C4)nc3c(F)c(-c4ccc(F)c5sc(N)c(C#N)c45)c(C(F)(F)F)cc23)[C@H]1C] | LCMS m/z [M + 1]: 732.3 1H NMR (400 MHz, CD3OD) δ 8.41 (s, 1H), 7.24 (dd, J = 8.1, 5.1 Hz, 1H), 7.03 (t, J = 8.9 Hz, 1H), 5.56 (d, J = 52.2 Hz, 1H), 4.97-5.04 (m, 1H), 4.58- 4.82 (m, 3H), 3.79-4.06 (m, 4H), 3.65 (s, 3H), 3.45- 3.61 (m, 2H), 2.56-2.75 (m, 3H), 2.28-2.49 (m, 6H), 2.07-2.21 (m, 1H), 1.04-1.21 (m, 6H). |
| 260a | 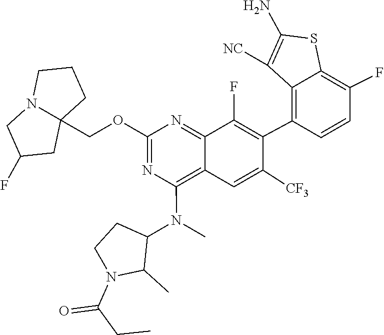 [SMILES: CCC(=O)N1CCC(N(C)c2nc(OCC34CCCN3CC(F)C4)nc3c(F)c(-c4ccc(F)c5sc(N)c(C#N)c45)c(C(F)(F)F)cc23)C1C] | |
| 261 | 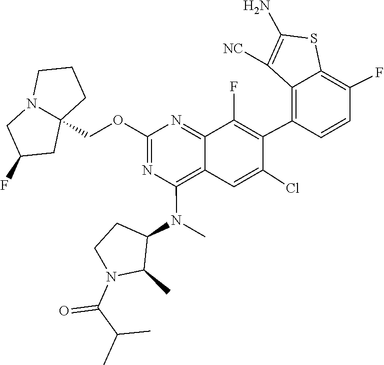 [SMILES: CC(C)C(=O)N1CC[C@@H](N(C)c2nc(OC[C@@]34CCCN3C[C@H](F)C4)nc3c(F)c(-c4ccc(F)c5sc(N)c(C#N)c45)c(Cl)cc23)[C@H]1C] | LCMS m/z [M + 1]: 746.3 1H NMR (400 MHz, CD3OD) δ 8.41 (s, 1H), 7.21- 7.27 (m, 1H), 7.03 (t, J = 8.9 Hz, 1H), 5.56 (d, J = 52.8 Hz, 1H), 4.96-5.04 (m, 1H), 4.59-4.82 (m, 3H), 3.82-4.07 (m, 4H), 3.62-3.73 (m, 4H), 3.43-3.50 (m, 1H), 2.55-2.83 (m, 4H), 2.30-2.48 (m, 4H), 2.13 (s, 1H), 1.03-1.20 (m, 9H). |
| 261a | 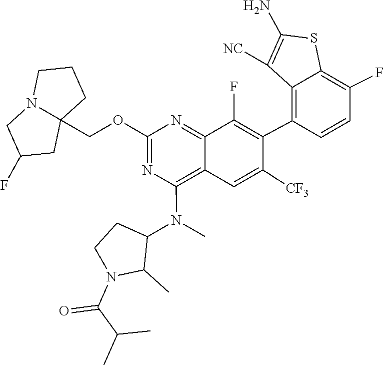 [SMILES: CC(C)C(=O)N1CCC(N(C)c2nc(OCC34CCCN3CC(F)C4)nc3c(F)c(-c4ccc(F)c5sc(N)c(C#N)c45)c(C(F)(F)F)cc23)C1C] | |
| 262 | 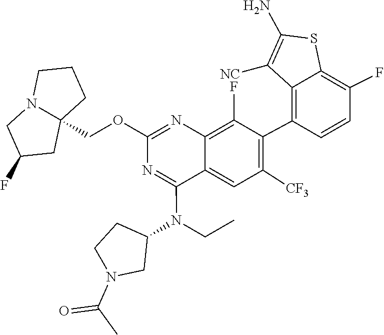 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@H]1CCN(C(C)=O)C1] | LCMS m/z [M + 1]: 718.3 1H NMR (400 MHz, CD3OD) δ 8.24 (s, 1H), 7.20- 7.23 (m, 1H), 7.01 (t, 1H), 5.36 (s, 0.5H), 5.23 (s, 0.5H), 4.98-5.06 (m, 1H), 4.21-4.30 (m, 2H), 3.41- 4.07 (m, 6H), 3.20-3.25 (m, 3H), 2.98-3.02 (m, 1H), 2.20-2.54 (m, 4H), 2.08-2.17 (m, 4H), 1.87-2.01 (m, 3H), 1.40-1.48 (m, 3H). |
| 262a | 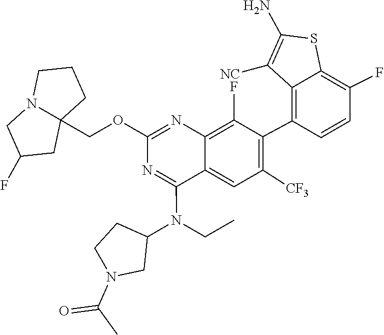 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(C)=O)C1] | |
| 263 | 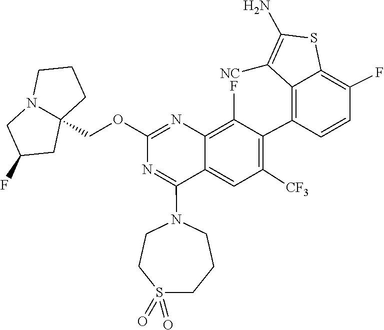 [SMILES: N#Cc1c(N)sc2c(F)ccc(-c3c(C(F)(F)F)cc4c(N5CCCS(=O)(=O)CC5)nc(OC[C@@]56CCCN5C[C@H](F)C6)nc4c3F)c12] | LCMS m/z [M + 1]: 711.2 1H NMR (400 MHz, CD3OD) δ 8.29 (s, 1H), 7.19- 7.23 (m, 1H), 7.01 (t, 1H), 5.37 (s, 0.5H), 5.23 (s, 0.5H), 4.21-4.36 (m, 6H), 3.69-3.70 (m, 2H), 3.32- 3.38 (m, 2H), 3.20-3.25 (m, 3H), 2.99-3.02 (m, 1H), 2.43-2.50 (m, 2H), 2.12-2.37 (m, 3H), 1.87-2.01 (m, 3H). |
| 263a | 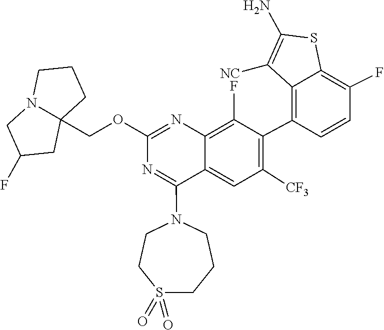 [SMILES: N#Cc1c(N)sc2c(F)ccc(-c3c(C(F)(F)F)cc4c(N5CCCS(=O)(=O)CC5)nc(OCC56CCCN5CC(F)C6)nc4c3F)c12] | |
| 264 | 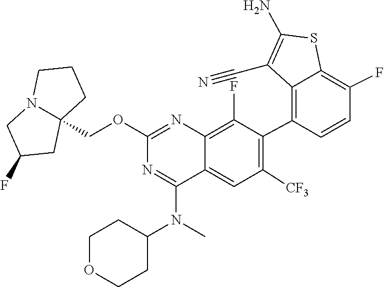 [SMILES: CN(c1nc(OC[C@@]23CCCN2C[C@H](F)C3)nc2c(F)c(-c3ccc(F)c4sc(N)c(C#N)c34)c(C(F)(F)F)cc12)C1CCOCC1] | LCMS m/z [M + 1]: 677.0 1H NMR (400 MHz, CD3OD) δ 8.273 (s, 1H), 7.198-7.232 (s, 1H), 6.989-7.031 (s, 1H), 5.382 (s, 0.5H), 5.248 (s, 0.5H), 4.234-4.312 (m, 2H), 4.081- 4.110 (m, 2H), 3.570-3.634 (m, 2H), 3.404 (m, 3H), 3.344 (s, 1H), 3.249 (m, 3H), 3.017-3.054 (s, 1H), 2.341-2.391 (m, 2H), 1.881-2.288 (m, 8H). |
| 264a | 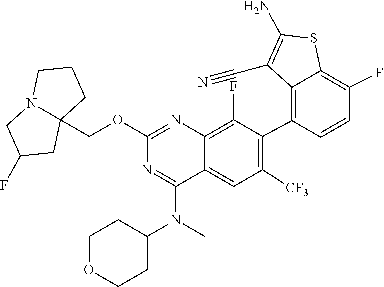 [SMILES: CN(c1nc(OCC23CCCN2CC(F)C3)nc2c(F)c(-c3ccc(F)c4sc(N)c(C#N)c34)c(C(F)(F)F)cc12)C1CCOCC1] | |
| 265 | 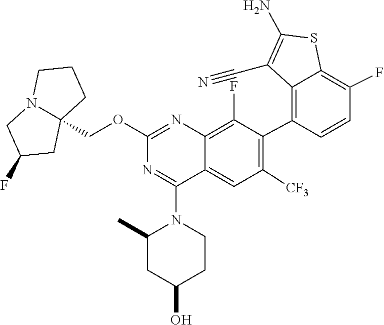 [SMILES: C[C@@H]1C[C@H](O)CCN1c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 677.1 1H NMR (400 MHz, CD3OD) δ 8.087-8.104 (s, 1H), 7.198-7.232 (s, 1H), 6.989-7.033 (s, 1H), 5.443 (s, 0.5H), 5.310 (s, 0.5H), 4.733 (s, 1H), 4.400 (m, 2H), 4.182 (m, 2H), 3.886 (s, 1H), 3.438-3.558 (m, 3H). 2.319-2.459 (m, 2H), 2.213-2.233 (s, 1H), 1.961- 2.097 (m, 5H), 2.233 (s, 1H), 1.838-1.871 (m, 2H), 1.688-1.716 (m, 3H). |
| 265a | 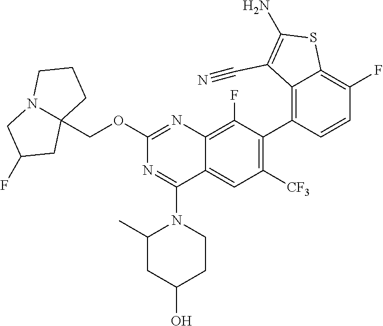 [SMILES: CC1CC(O)CCN1c1nc(OCC23CCCN2CC(F)C3)nc2c(F)c(-c3ccc(F)c4sc(N)c(C#N)c34)c(C(F)(F)F)cc12] | |
| 266 | 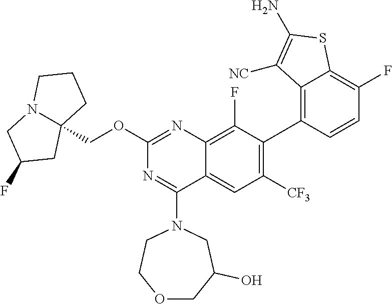 [SMILES: N#Cc1c(N)sc2c(F)ccc(-c3c(C(F)(F)F)cc4c(N5CCOCC(O)C5)nc(OC[C@@]56CCCN5C[C@H](F)C6)nc4c3F)c12] | LCMS m/z [M + 1]: 679.2 1H NMR (400 MHz, CD3OD) δ 8.59-8.45 (m, 1H), 7.25-7.17 (m, 1H), 7.06-6.96 (m, 1H), 5.40-5.35 (s, 0.5H), 5.27-5.22 (s, 0.5H), 4.37-3.90 (m, 9H), 3.74- 3.65 (m, 1H), 3.30-3.16 (m, 4H), 3.08-2.98 (m, 1H), 2.39-2.13 (m, 3H), 2.05-1.86 (m, 3H). |
| 266a | 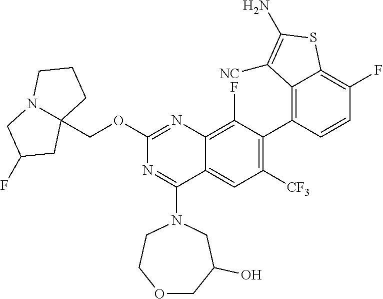 [SMILES: N#Cc1c(N)sc2c(F)ccc(-c3c(C(F)(F)F)cc4c(N5CCOCC(O)C5)nc(OCC56CCCN5CC(F)C6)nc4c3F)c12] | |
| 267 | 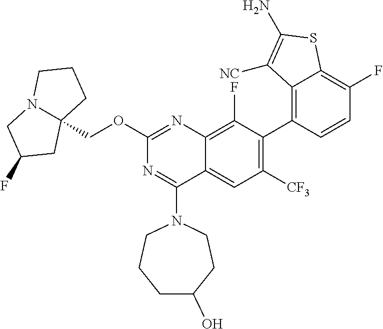 [SMILES: N#Cc1c(N)sc2c(F)ccc(-c3c(C(F)(F)F)cc4c(N5CCCC(O)CC5)nc(OC[C@@]56CCCN5C[C@H](F)C6)nc4c3F)c12] | LCMS m/z [M + 1]: 677.2 1H NMR (400 MHz, CD3OD) δ 8.31 (s, 1H), 7.22- 7.19 (m, 1H), 7.03-6.99 (m, 1H), 5.48 (s, 0.5H), 5.34 (s, 0.5H), 4.48-4.38 (m, 2H), 4.11-3.96 (m, 5H), 3.66-3.53 (m, 3H), 3.24-3.17 (m, 1H), 2.49-1.77 (m, 12H). |
| 267a | 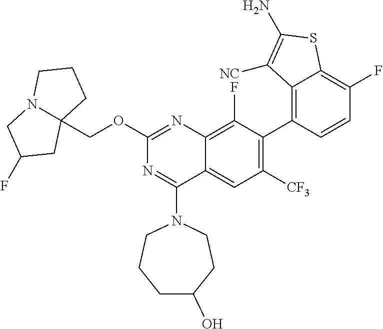 [SMILES: N#Cc1c(N)sc2c(F)ccc(-c3c(C(F)(F)F)cc4c(N5CCCC(O)CC5)nc(OCC56CCCN5CC(F)C6)nc4c3F)c12] | |
| 268 | 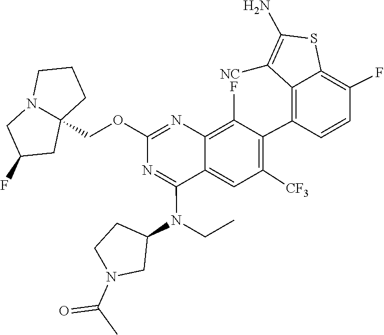 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@@H]1CCN(C(C)=O)C1] | LCMS m/z [M + 1]: 718.2 1H NMR (400 MHz, CD3OD) δ 8.24 (s, 1H), 7.18- 7.25 (m, 1H), 7.01 (t, 1H), 5.37 (s, 0.5H), 5.24 (s, 0.5H), 4.98-5.09 (m, 1H), 4.19-4.35 (m, 2H), 3.72- 4.05 (m, 4H), 3.53-4.61 (m, 1H), 3.38-3.50 (m, 1H), 3.19-3.28 (m, 3H), 2.98-3.07 (m, 1H), 2.05-2.60 (m, 8H), 1.95-2.04 (m, 2H), 1.84-1.93 (m, 1H), 1.35-1.52 (m, 3H). |
| 268a | 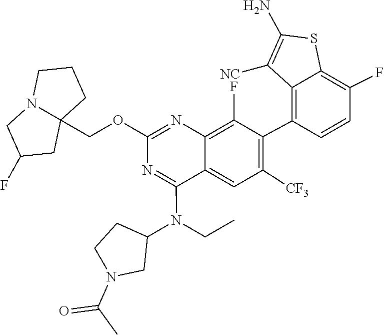 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(C)=O)C1] | |
| 269 | 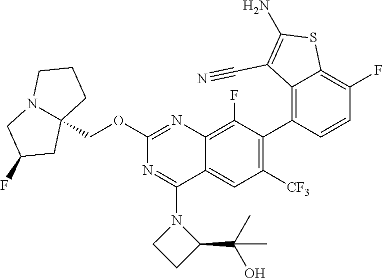 [SMILES: CC(C)(O)[C@H]1CCN1c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 677.3 1H NMR (400 MHz, CD3OD) δ 8.13 (s, 1H), 7.20- 7.17 (m, 1H), 7.00 (t, 1H), 5.38 (s, 0.5H), 5.24 (s, 0.5H), 4.84-4.78 (m, 2H), 4.68-4.62 (m, 1H), 4.30- 4.22 (m, 2H), 3.28-3.19 (m, 3H), 3.07-3.02 (m, 1H), 2.62-2.54 (m, 1H), 2.45-2.08 (m, 4H), 2.05-1.89 (m, 3H), 1.36-1.30 (m, 3H), 1.28-1.21 (m, 3H) |
| 269a | 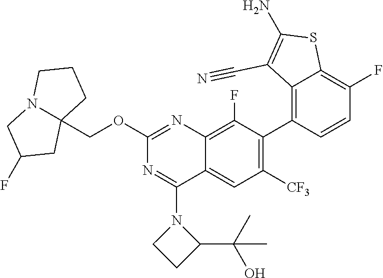 [SMILES: CC(C)(O)C1CCN1c1nc(OCC23CCCN2CC(F)C3)nc2c(F)c(-c3ccc(F)c4sc(N)c(C#N)c34)c(C(F)(F)F)cc12] | |
| 270 | 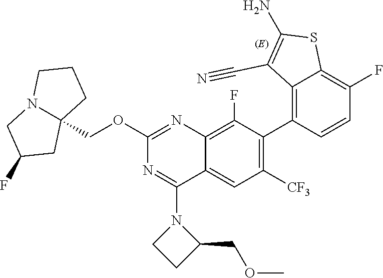 [SMILES: COC[C@H]1CCN1c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 663.0 1H NMR (400 MHz, CD3OD) δ 8.09-8.08 (m, 1H), 7.20-7.16 (m, 1H), 7.02-6.97 (m, 1H), 5.40 (s, 0.5H), 5.26 (s, 0.5H), 5.03 (s, 1H), 4.82-4.73 (m, 1H), 4.66- 4.54 (m, 1H), 4.43-4.20 (m, 2H), 4.09-4.06 (m, 1H), 3.77-3.72 (m, 1H), 3.43-3.40 (m, 3H), 3.33-3.31 (m, 2H), 3.28-3.20 (m, 1H), 3.08-3.07 (m, 1H), 2.73-2.49 (m, 2H), 2.44-1.89 (m, 6H). |
| 270a | 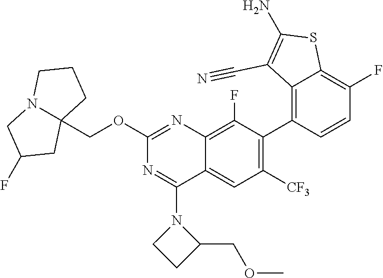 [SMILES: COCC1CCN1c1nc(OCC23CCCN2CC(F)C3)nc2c(F)c(-c3ccc(F)c4sc(N)c(C#N)c34)c(C(F)(F)F)cc12] | |
| 271 | 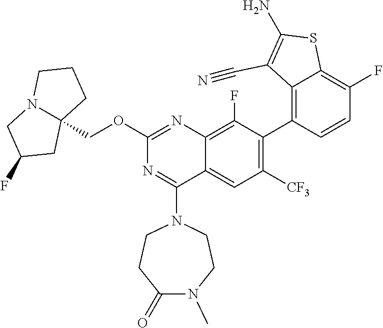 [SMILES: CN1CCN(c2nc(OC[C@@]34CCCN3C[C@H](F)C4)nc3c(F)c(-c4ccc(F)c5sc(N)c(C#N)c45)c(C(F)(F)F)cc23)CCC1=O] | LCMS m/z [M + 1]: 690.0 1HNMR (400 MHz, CD3OD) δ 8.13 (s, 1H), 7.18- 7.26 (m, 1H), 7.01 (t, 1H), 5.38 (s, 0.5H), 5.25 (s, 0.5H), 4.23-4.37 (m, 2H), 3.96-4.20 (m, 4H), 3.73- 3.90 (m, 2H), 3.33 (s, 1H), 3.20-3.28 (m, 2H), 2.97- 3.12 (m, 3H), 2.92 (s, 3H), 2.13-2.37 (m, 3H), 1.82- 2.05 (m, 3H). |
| 271a | 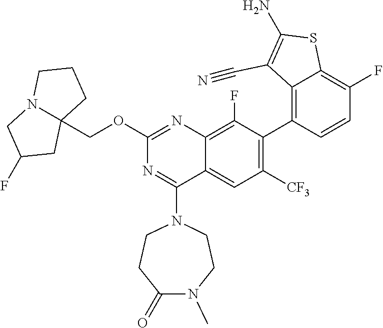 [SMILES: CN1CCN(c2nc(OCC34CCCN3CC(F)C4)nc3c(F)c(-c4ccc(F)c5sc(N)c(C#N)c45)c(C(F)(F)F)cc23)CCC1=O] | |
| 272 | 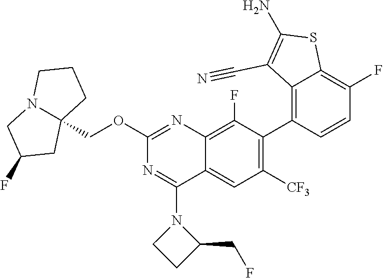 [SMILES: N#Cc1c(N)sc2c(F)ccc(-c3c(C(F)(F)F)cc4c(N5CC[C@@H]5CF)nc(OC[C@@]56CCCN5C[C@H](F)C6)nc4c3F)c12] | LCMS m/z [M + 1]: 651.2 1HNMR (400 MHz, CD3OD) δ 8.11 (s, 1H), 7.15- 7.25 (m, 1H), 7.01 (t, 1H), 5.51 (s, 0.5H), 5.38 (s, 0.5H), 5.02-5.25 (m, 2H), 4.80-4.86 (m, 1H), 4.58- 4.77 (m, 2H), 4.38-4.55 (m, 2H), 3.57-3.83 (m, 3H), 3.23-3.28 (m, 1H), 2.37-2.78 (m, 4H), 2.12-2.35 (m, 3H) 1.97-2.10 (m, 1H). |
| 272a | 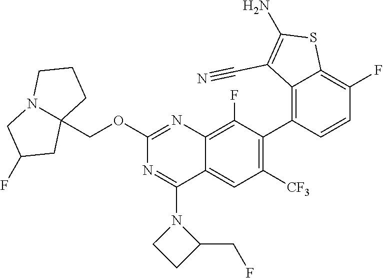 [SMILES: N#Cc1c(N)sc2c(F)ccc(-c3c(C(F)(F)F)cc4c(N5CCC5CF)nc(OCC56CCCN5CC(F)C6)nc4c3F)c12] | |
| 273 | 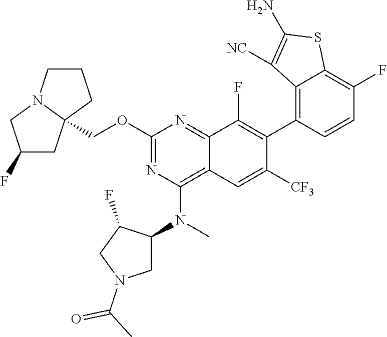 [SMILES: CC(=O)N1C[C@H](F)[C@@H](N(C)c2nc(OC[C@@]34CCCN3C[C@H](F)C4)nc3c(F)c(-c4ccc(F)c5sc(N)c(C#N)c45)c(C(F)(F)F)cc23)C1] | LCMS m/z [M + 1]: 722.2 1H NMR (400 MHz, CD3OD) δ 8.36 (s, 1H), 7.20- 7.23 (m, 1H), 7.01 (t, 1H), 5.71-5.72 (m, 0.5H), 5.57- 5.60 (m, 0.5H), 5.38 (s, 0.5H), 5.19-5.24 (m, 1.5H), 4.06-4.32 (m, 4H), 3.75-3.94 (m, 2H), 3.48-3.53 (m, 3H), 3.23-3.27 (m, 3H), 3.00-3.04 (m, 1H), 2.10-2.34 (m, 6H), 1.89-2.04 (m, 3H). |
| 273a | 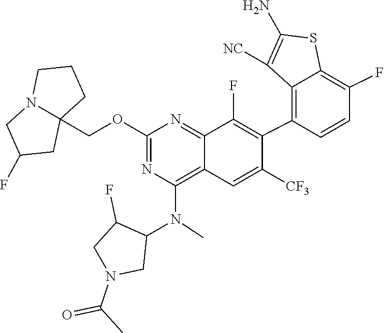 [SMILES: CC(=O)N1CC(F)C(N(C)c2nc(OCC34CCCN3CC(F)C4)nc3c(F)c(-c4ccc(F)c5sc(N)c(C#N)c45)c(C(F)(F)F)cc23)C1] | |
| 274 | 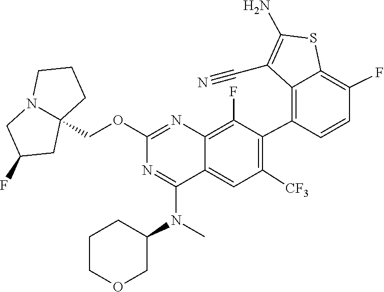 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@@H]1CCCOC1] | LCMS m/z [M + 1]: 677.1 1H NMR (400 MHz, CD3OD) δ 8.27 (s, 1H), 7.19- 7.23 (m, 1H), 6.98-7.02 (m, 1H), 5.37 (s, 0.5H), 5.23 (s, 0.5H), 4.71-4.74 (m, 1H), 4.31-4.34 (d, 1H), 4.23- 4.25 (d, 1H), 4.11-4.15 (m, 1H), 3.91-3.93 (d, 1H), 3.63-3.71 (m, 1H), 3.44-3.48 (m, 1H), 3.41 (d, 3H), 3.15-3.24 (m, 3H), 2.98-3.04 (m, 1H), 2.20-2.39 (m, 2H) 2.07-2.14 (m, 3H) 1.85-2.03 (m, 5H). |
| 274a | 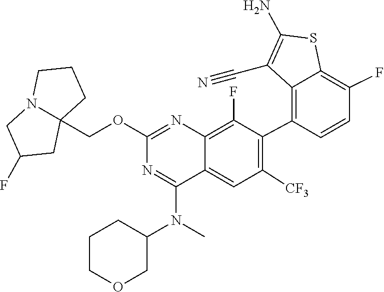 [SMILES: CN(c1nc(OCC23CCCN2CC(F)C3)nc2c(F)c(-c3ccc(F)c4sc(N)c(C#N)c34)c(C(F)(F)F)cc12)C1CCCOC1] | |
| 275 | 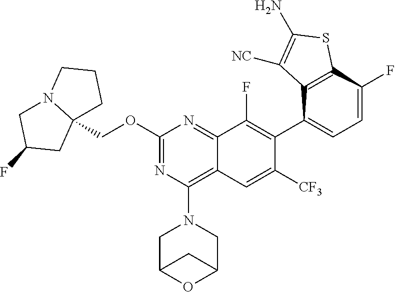 | LCMS m/z [M + 1]: 661.3 1H NMR (400 MHz, CD3OD) δ 8.59 (s, 1H), 7.16- 7.24 (m, 1H), 7.01 (t, J = 9.2 Hz, 1H), 5.22-5.37 (m, 1H), 4.46-4.59 (m, 3H), 4.21-4.39 (m, 4H), 3.12-3.26 (m, 4H), 2.96-3.04 (m, 1H), 2.09-2.40 (m, 4H), 1.84- 2.03 (m, 4H). |
| 276 | 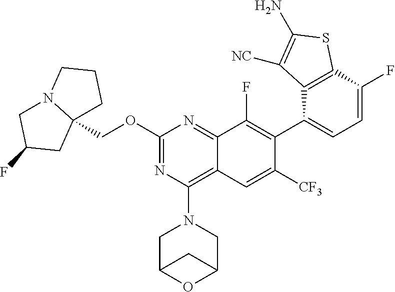 | LCMS m/z [M + 1]: 661.3 1H NMR (400 MHz, CD3OD) δ 8.59 (s, 1H), 7.16- 7.24 (m, 1H), 7.01 (t, J = 9.2 Hz, 1H), 5.21-5.38 (m, 1H), 4.44-4.64 (m, 3H), 4.18-4.42 (m, 4H), 3.10-3.29 (m, 4H), 2.93-3.03 (m, 1H), 1.81-2.40 (m, 8H). |
| 276a | 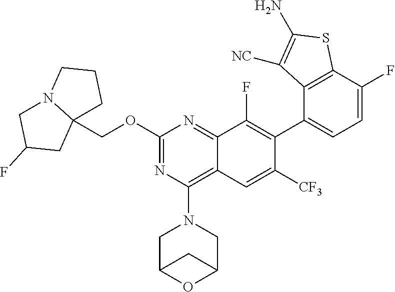 [SMILES: N#Cc1c(N)sc2c(F)ccc(-c3c(C(F)(F)F)cc4c(N5CC6CC(C5)O6)nc(OCC56CCCN5CC(F)C6)nc4c3F)c12] | |
| 277 | 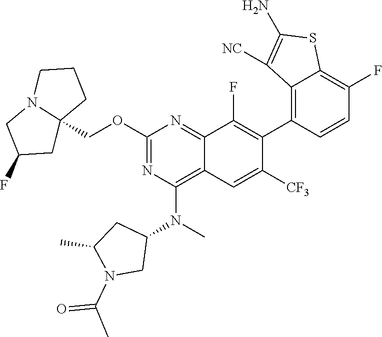 [SMILES: CC(=O)N1C[C@@H](N(C)c2nc(OC[C@@]34CCCN3C[C@H](F)C4)nc3c(F)c(-c4ccc(F)c5sc(N)c(C#N)c45)c(C(F)(F)F)cc23)C[C@H]1C] | LCMS m/z [M + 1]: 718.3 1H NMR (400 MHz, CD3OD) δ 8.29 (s, 1H), 7.18- 7.25 (m, 1H), 7.00 (t, 1H), 5.36 (s, 0.5H), 5.23 (s, 0.5H), 4.97-5.15 (m, 1H), 4.05-4.42 (m, 4H), 3.59- 3.75 (m, 1H), 3.46 (s, 3H), 3.17-3.29 (m, 3H), 2.97- 3.06 (m, 1H), 2.56-2.83 (m, 1H), 2.08-2.38 (m, 6H), 1.82-2.05 (m, 4H), 1.38-1.50 (m, 3H). |
| 277a | 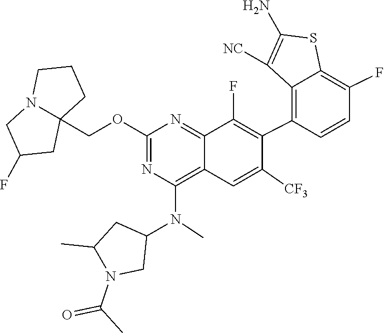 [SMILES: CC(=O)N1CC(N(C)c2nc(OCC34CCCN3CC(F)C4)nc3c(F)c(-c4ccc(F)c5sc(N)c(C#N)c45)c(C(F)(F)F)cc23)CC1C] | |
| 278 | 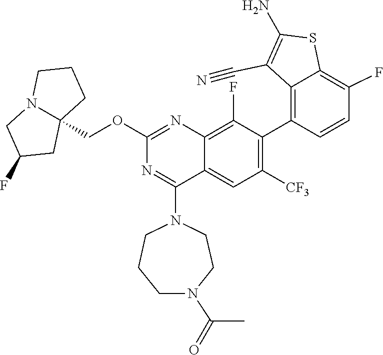 [SMILES: CC(=O)N1CCCN(c2nc(OC[C@@]34CCCN3C[C@H](F)C4)nc3c(F)c(-c4ccc(F)c5sc(N)c(C#N)c45)c(C(F)(F)F)cc23)CC1] | LCMS m/z [M + 1]: 704.1 1H NMR (400 MHz, CD3OD) δ 8.25 (d, 1H), 7.15- 7.25 (m, 1H), 7.00 (t, 1H), 5.37 (s, 0.5H), 5.24 (s, 0.5H), 4.19-4.34 (m, 4H), 3.71-4.15 (m, 5H), 3.57- 3.70 (m, 1H), 3.12-3.28 (m, 3H), 2.98-3.06 (s, 1H), 2.12-2.45 (m, 5H), 2.09 (d, 3H), 1.96-2.03 (m, 2H), 1.84-1.94 (s, 1H). |
| 278a | 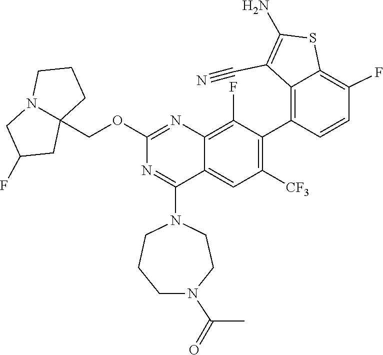 [SMILES: CC(=O)N1CCCN(c2nc(OCC34CCCN3CC(F)C4)nc3c(F)c(-c4ccc(F)c5sc(N)c(C#N)c45)c(C(F)(F)F)cc23)CC1] | |
| 279 | 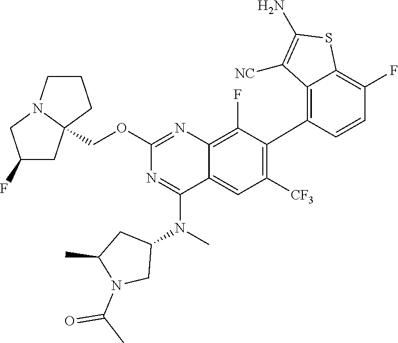 [SMILES: CC(=O)N1C[C@@H](N(C)c2nc(OC[C@@]34CCCN3C[C@H](F)C4)nc3c(F)c(-c4ccc(F)c5sc(N)c(C#N)c45)c(C(F)(F)F)cc23)C[C@@H]1C] | LCMS m/z [M + 1]: 718.3 1H NMR (400 MHz, CD3OD) δ 8.34-8.23 (m, 1H), 7.25-7.18 (m, 1H), 7.04-6.97 (m, 1H), 5.54-5.42 (m, 1H), 5.39-5.35 (s, 0.5H), 5.27-5.21 (s, 0.5H), 4.42- 4.22 (m, 3H), 4.11-3.85 (m, 1H), 3.76-3.61 (m, 1H), 3.45-3.38 (m, 3H), 3.26-3.20 (m, 2H), 3.06-2.98 (m, 1H), 2.65-2.48 (m, 1H), 2.38-1.81 (m, 11H), 1.32- 1.28 (m, 3H). |
| 279a | 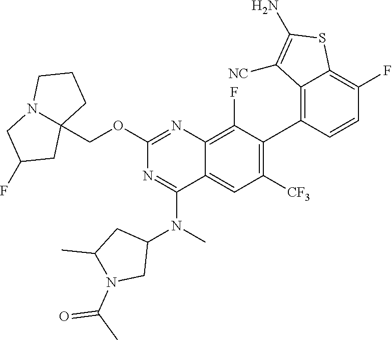 [SMILES: CC(=O)N1CC(N(C)c2nc(OCC34CCCN3CC(F)C4)nc3c(F)c(-c4ccc(F)c5sc(N)c(C#N)c45)c(C(F)(F)F)cc23)CC1C] | |
| 280 | 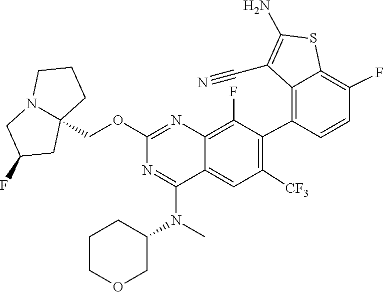 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@H]1CCCOC1] | LCMS m/z [M + 1]: 677.2 1H NMR (400 MHz, CD3OD) δ 8.49 (s, 1H), 8.31 (s, 1H), 7.21 (t, 1H), 7.01 (t, 1H) 5.52 (s, 0.5H), 5.38 (s, 0.5H), 4.73-4.80 (m, 1H), 4.54-4.56 (m, 2H), 4.11-4.14 (m, 1H), 3.93 (d, 1H), 3.62-3.78 (m, 4H), 3.46-3.55 (m, 1H), 3.44 (s, 3H), 3.23-3.28 (m, 1H), 2.40-2.61 (m, 2H), 2.01-2.36 (m, 6H), 1.81-1.90 (m, 2H). |
| 280a | 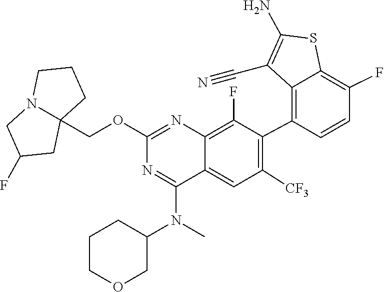 [SMILES: CN(c1nc(OCC23CCCN2CC(F)C3)nc2c(F)c(-c3ccc(F)c4sc(N)c(C#N)c34)c(C(F)(F)F)cc12)C1CCCOC1] | |
| 281 | 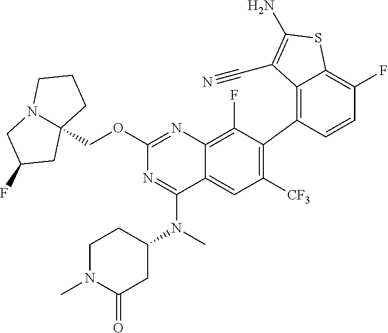 [SMILES: CN1CC[C@H](N(C)c2nc(OC[C@@]34CCCN3C[C@H](F)C4)nc3c(F)c(-c4ccc(F)c5sc(N)c(C#N)c45)c(C(F)(F)F)cc23)CC1=O] | LCMS m/z [M + 1]: 704.2 1H NMR (400 MHz, CD3OD) δ 8.40 (d, 1H), 7.18- 7.23 (m, 1H), 7.00 (t, 1H), 5.36 (s, 0.5H), 5.23 (s, 0.5H), 4.11-4.30 (m, 2H), 3.47-3.64 (m, 4H), 3.33- 3.41 (m, 2H), 3.20-4.27 (m, 4H), 3.02 (s, 3H), 2.16- 2.35 (m, 4H), 1.95-2.13 (m, 5H), 1.86-1.90 (m, 1H). |
| 281a | 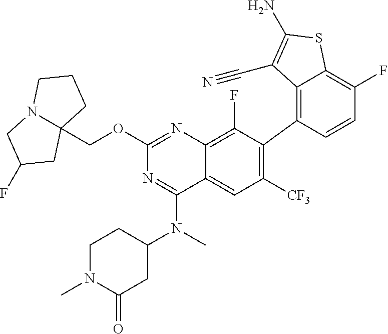 [SMILES: CN1CCC(N(C)c2nc(OCC34CCCN3CC(F)C4)nc3c(F)c(-c4ccc(F)c5sc(N)c(C#N)c45)c(C(F)(F)F)cc23)CC1=O] | |
| 282 | 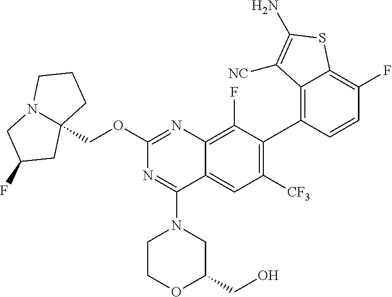 [SMILES: N#Cc1c(N)sc2c(F)ccc(-c3c(C(F)(F)F)cc4c(N5CCO[C@@H](CO)C5)nc(OC[C@@]56CCCN5C[C@H](F)C6)nc4c3F)c12] | LCMS m/z [M + 1]: 679.3 1H NMR (400 MHz, CD3OD) δ 8.14 (s, 1H), 7.20 (t, 1H), 7.00 (t, 1H), 5.36 (s, 0.5H), 5.23 (s, 0.5H), 4.54- 4.45 (m, 1H), 4.39-4.22 (m, 3H), 4.05-4.02 (m, 1H), 3.84-3.75 (m, 2H), 3.66-3.55 (m, 3H), 3.40-3.34 (m, 1H), 3.27-3.15 (m, 3H), 3.06-2.98 (m, 1H), 2.38-2.12 (m, 3H), 2.03-1.87 (m, 3H). |
| 282a | 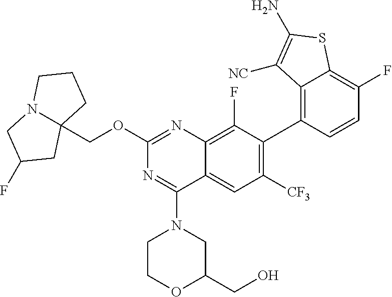 [SMILES: N#Cc1c(N)sc2c(F)ccc(-c3c(C(F)(F)F)cc4c(N5CCOC(CO)C5)nc(OCC56CCCN5CC(F)C6)nc4c3F)c12] | |
| 283 | 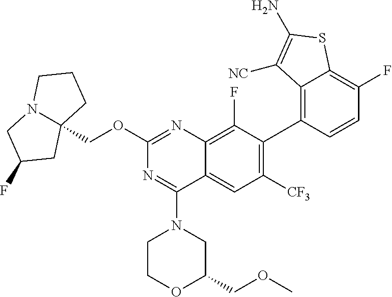 [SMILES: COC[C@H]1CN(c2nc(OC[C@@]34CCCN3C[C@H](F)C4)nc3c(F)c(-c4ccc(F)c5sc(N)c(C#N)c45)c(C(F)(F)F)cc23)CCO1] | LCMS m/z [M + 1]: 693.3 1H NMR (400 MHz, CD3OD) δ 8.13 (s, 1H), 7.20 (t, 1H), 7.00 (t, 1H), 5.36 (s, 0.5H), 5.22 (s, 0.5H), 4.50- 4.30 (m, 3H), 4.24-4.21 (m, 1H), 4.06-4.01 (m, 1H), 3.90-3.77 (m, 2H), 3.64-3.47 (m, 3H), 3.40-3.34 (m, 4H), 3.27-3.18 (m, 3H), 3.01-2.97 (m, 1H), 2.37-2.11 (m, 3H), 2.03-1.87 (m, 3H). |
| 283a | 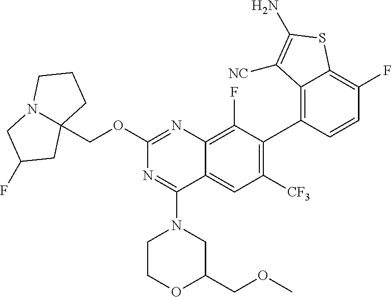 [SMILES: COCC1CN(c2nc(OCC34CCCN3CC(F)C4)nc3c(F)c(-c4ccc(F)c5sc(N)c(C#N)c45)c(C(F)(F)F)cc23)CCO1] | |
| 284 | 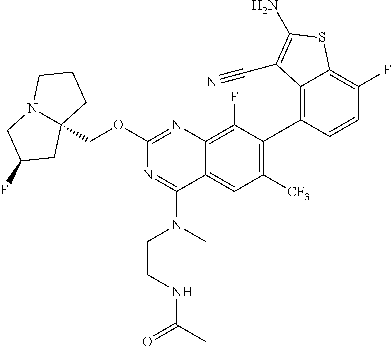 [SMILES: CC(=O)NCCN(C)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 678.2 1H NMR (400 MHz, CD3OD) δ 8.37 (s, 1H), 7.23- 7.18 (m, 1H), 7.03-6.98 (m, 1H), 5.37 (s, 0.5H), 5.24 (s, 0.5H), 4.34-4.29 (m, 1H), 4.25-4.20 (m, 1H), 4.02 (t, 2H), 3.67-3.56 (m, 5H), 3.28-3.16 (m, 3H), 3.06- 2.99 (m, 1H), 2.40-2.05 (m, 4H), 2.02-1.95 (m, 2H), 1.88 (s, 3H). |
| 284a | 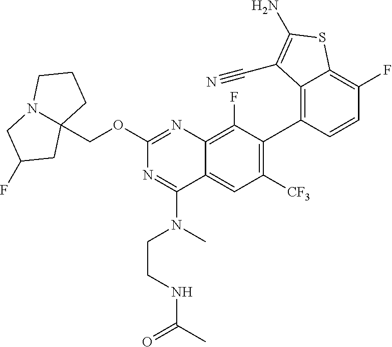 [SMILES: CC(=O)NCCN(C)c1nc(OCC23CCCN2CC(F)C3)nc2c(F)c(-c3ccc(F)c4sc(N)c(C#N)c34)c(C(F)(F)F)cc12] | |
| 285 | 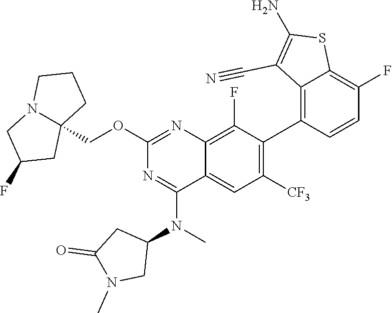 [SMILES: CN1C[C@H](N(C)c2nc(OC[C@@]34CCCN3C[C@H](F)C4)nc3c(F)c(-c4ccc(F)c5sc(N)c(C#N)c45)c(C(F)(F)F)cc23)CC1=O] | LCMS m/z [M + 1]: 690.3 1H NMR (400 MHz, CD3OD) δ 8.28 (s, 1H), 7.23- 7.19 (m, 1H), 7.01 (t, 1H), 5.42 (s, 0.5H), 5.33 (t, 1H), 5.29 (s, 0.5H), 4.37-4.24 (m, 2H), 4.00-3.93 (m, 1H), 3.73-3.65 (m, 1H), 3.47 (s, 3H), 3.39-3.35 (m, 1H), 3.13-3.06 (m, 1H), 2.93 (s, 3H), 2.85-2.79 (m, 1H), 2.45-2.16 (m, 4H), 2.08-1.91 (m, 4H), 1.62-1.56 (m, 1H). |
| 285a | 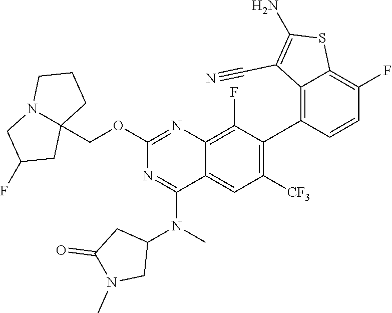 [SMILES: CN1CC(N(C)c2nc(OCC34CCCN3CC(F)C4)nc3c(F)c(-c4ccc(F)c5sc(N)c(C#N)c45)c(C(F)(F)F)cc23)CC1=O] | |
| 286 | 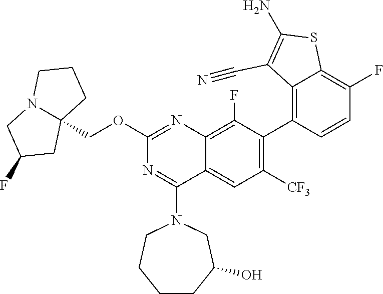 [SMILES: N#Cc1c(N)sc2c(F)ccc(-c3c(C(F)(F)F)cc4c(N5CCCC[C@@H](O)C5)nc(OC[C@@]56CCCN5C[C@H](F)C6)nc4c3F)c12] | LCMS m/z [M + 1]: 677.1 1H NMR (400 MHz, CD3OD) & 8.39-8.42 (s, 1H), 7.18-7.22 (m, 1H), 6.98-7.02 (m, 1H), 5.37 (s, 0.5H), 5.23 (s, 0.5H), 4.30-4.39 (m, 2H), 4.19-4.25 (m, 2H), 4.03-4.07 (m, 2H), 3.69-3.76 (m, 1H), 3.33 (m, 1H), 3.22-3.26 (m, 2H), 2.99-3.03 (m, 1H), 2.13-2.37 (m, 4H), 1.89-2.03 (m, 6H), 1.64-1.67 (m, 1H), 1.46-1.51 (m, 1H). |
| 286a | 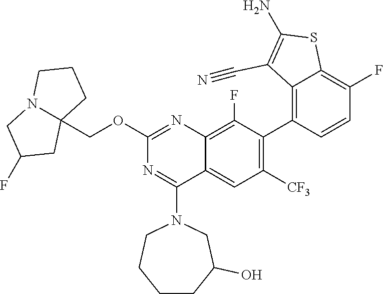 [SMILES: N#Cc1c(N)sc2c(F)ccc(-c3c(C(F)(F)F)cc4c(N5CCCCC(O)C5)nc(OCC56CCCN5CC(F)C6)nc4c3F)c12] | |
| 287 | 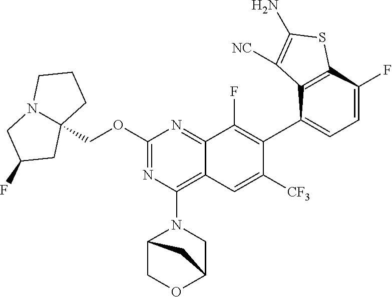 | LCMS m/z [M + 1]: 661.2 1H NMR (400 MHz, Methanol-d4) δ 8.30 (s, 1H), 7.20-7.24 (m, 1H), 7.01 (d, J = 9.6 Hz, 1H), 5.53 (s, 1H), 5.41 (t, J = 54 Hz, 1H), 4.21-4.35 (m, 3H), 3.98- 4.10 (m, 3H), 3.31-3.38 (m, 1H), 3.21-3.27 (m, 3H), 3.01-3.07 (m, 1H), 1.88-2.37 (m, 8H). |
| 288 | 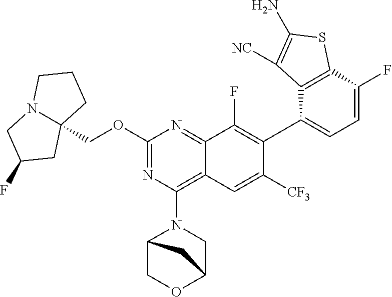 | LCMS m/z [M + 1]: 661.2 1H NMR (400 MHz, Methanol-d4) δ 8.30 (s, 1H), 7.20-7.24 (m, 1H), 7.01 (d, J = 9.3 Hz, 1H), 5.50 (s, 1H), 5.41 (t, J = 53.7 Hz, 1H), 4.27-4.39 (m, 3H), 3.99-4.11 (m, 3H), 3.31-3.38 (m, 1H), 3.21-3.27 (m, 3H), 3.01-3.07 (m, 1H), 1.88-2.37 (m, 8H). |
| 288a | 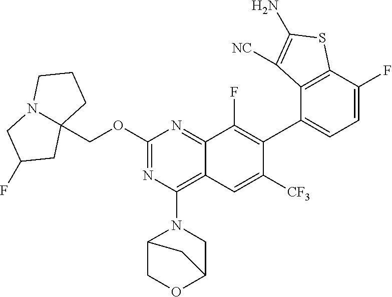 [SMILES: N#Cc1c(N)sc2c(F)ccc(-c3c(C(F)(F)F)cc4c(N5CC6CC5CO6)nc(OCC56CCCN5CC(F)C6)nc4c3F)c12] | |
| 289 | 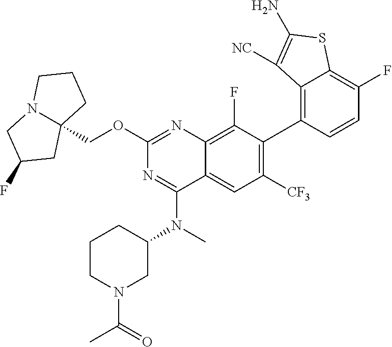 [SMILES: CC(=O)N1CCC[C@H](N(C)c2nc(OC[C@@]34CCCN3C[C@H](F)C4)nc3c(F)c(-c4ccc(F)c5sc(N)c(C#N)c45)c(C(F)(F)F)cc23)C1] | LCMS m/z [M + 1]: 718.1 1H NMR (400 MHz, CD3OD) δ 8.26 (m, 1H), 7.18- 7.24 (m, 1H), 7.00 (t, 1H), 5.38 (s, 0.5H), 5.24 (s, 0.5H), 4.49-4.74 (m, 2H), 4.15-4.45 (m, 3H), 3.87- 4.02 (m, 1H), 3.43-3.50 (m, 3H), 3.14-3.27 (m, 3H), 2.83-3.06 (m, 2H), 1.84-2.30 (m, 13H). |
| 289a | 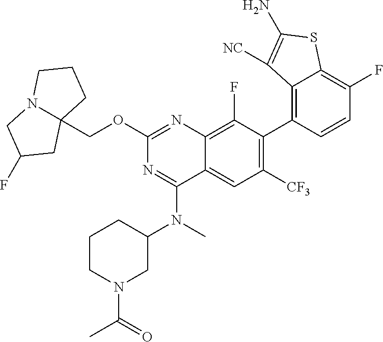 [SMILES: CC(=O)N1CCCC(N(C)c2nc(OCC34CCCN3CC(F)C4)nc3c(F)c(-c4ccc(F)c5sc(N)c(C#N)c45)c(C(F)(F)F)cc23)C1] | |
| 290 | 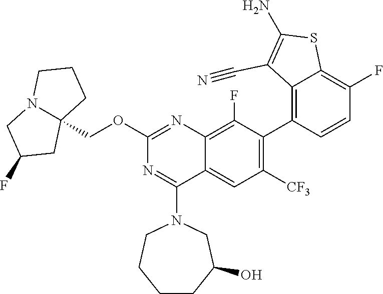 [SMILES: N#Cc1c(N)sc2c(F)ccc(-c3c(C(F)(F)F)cc4c(N5CCCC[C@H](O)C5)nc(OC[C@@]56CCCN5C[C@H](F)C6)nc4c3F)c12] | LCMS m/z [M + 1]: 677.0 1H NMR (400 MHz, CD3OD) δ 8.40-8.43 (d, 1H), 7.18-7.22 (m, 1H), 6.98-7.02 (m, 1H), 5.37 (s, 0.5H), 5.24 (s, 0.5H), 4.30-4.40 (m, 2H), 4.18-4.26 (m, 2H), 4.06-4.07 (m, 2H), 3.69-3.76 (m, 1H), 3.23-3.27 (m, 3H), 3.00-3.03 (m, 1H), 2.14-2.39 (m, 4H), 1.97-2.03 (m, 4H), 1.88-1.94 (m, 2H), 1.65-1.67 (m, 1H), 1.45- 1.51 (m, 1H). |
| 290a | 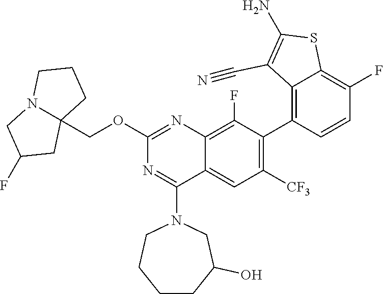 [SMILES: N#Cc1c(N)sc2c(F)ccc(-c3c(C(F)(F)F)cc4c(N5CCCCC(O)C5)nc(OCC56CCCN5CC(F)C6)nc4c3F)c12] | |
| 291 | 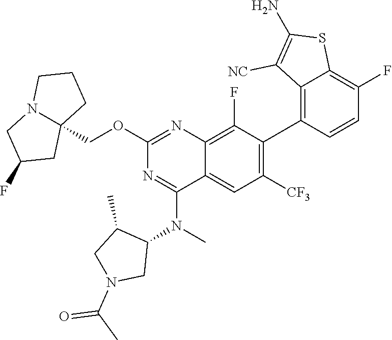 | LCMS m/z [M + 1]: 718.23 1H NMR (400 MHz, CD3OD) δ 8.32 (s, 1H), 7.23- 7.18 (m, 1H), 7.05-6.96 (m, 1H), 5.48-5.39 (m, 1H), 5.41 (s, 0.5H), 5.23 (s, 0.5H), 4.34-4.21 (m, 2H), 4.14-3.98 (m, 1H), 3.95-3.79 (m, 2H), 3.48-3.39 (m, 3H), 3.27-3.16 (m, 3H), 3.07-2.92 (m, 2H), 2.39-1.82 (m, 10H), 1.17-1.10 (m, 3H). |
| 291a | 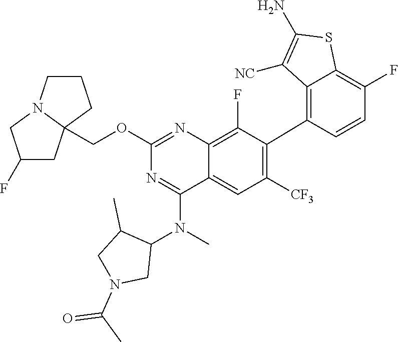 [SMILES: CC(=O)N1CC(C)C(N(C)c2nc(OCC34CCCN3CC(F)C4)nc3c(F)c(-c4ccc(F)c5sc(N)c(C#N)c45)c(C(F)(F)F)cc23)C1] | |
| 292 | 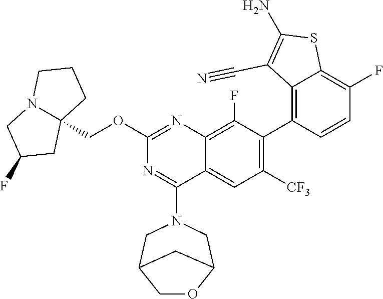 [SMILES: N#Cc1c(N)sc2c(F)ccc(-c3c(C(F)(F)F)cc4c(N5CC6COC(C6)C5)nc(OC[C@@]56CCCN5C[C@H](F)C6)nc4c3F)c12] | LCMS m/z [M + 1]: 675.0 1H NMR (400 MHz, CD3OD) δ 8.25 (d, 1H), 7.24- 7.17 (m, 1H), 7.04-6.97 (m, 1H), 5.46 (s, 0.5H), 5.33 (s, 0.5H), 4.82-4.76 (m, 1H), 4.57-4.34 (m, 4H), 3.99-3.93 (m, 1H), 3.85-3.79 (m, 1H), 3.76-3.56 (m, 3H), 3.54-3.46 (m, 2H), 3.22-3.16 (m, 1H), 2.71 (s, 1H), 2.59-2.20 (m, 4H), 2.19-2.02 (m, 4H). |
| 292a | 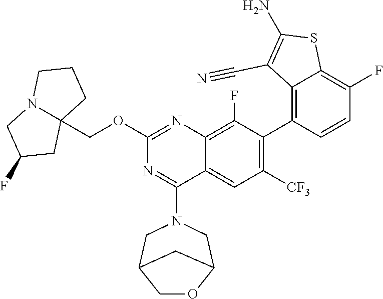 [SMILES: N#Cc1c(N)sc2c(F)ccc(-c3c(C(F)(F)F)cc4c(N5CC6COC(C6)C5)nc(OCC56CCCN5C[C@H](F)C6)nc4c3F)c12] | |
| 293 | 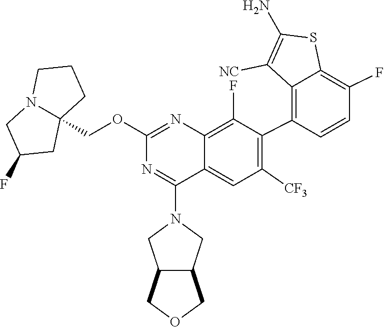 | LCMS m/z [M + 1]: 675.1 1H NMR (400 MHz, CD3OD) δ 8.42 (s, 1H), 7.22- 7.16 (m, 1H), 7.00 (t, 1H), 5.38 (s, 0.5H), 5.25 (s, 0.5H), 4.42-4.30 (m, 3H), 4.28-4.22 (m, 1H), 4.09- 4.00 (m, 2H), 4.00-3.93 (m, 2H), 3.85-3.78 (m, 2H), 3.25-3.18 (m, 3H), 3.08-2.99 (m, 1H), 2.45-2.12 (m, 4H), 2.10-1.85 (m, 4H) |
| 293a | 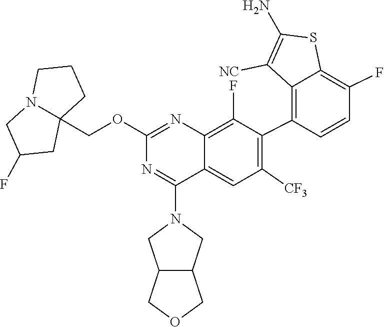 [SMILES: N#Cc1c(N)sc2c(F)ccc(-c3c(C(F)(F)F)cc4c(N5CC6COCC6C5)nc(OCC56CCCN5CC(F)C6)nc4c3F)c12] | |
| 294 | 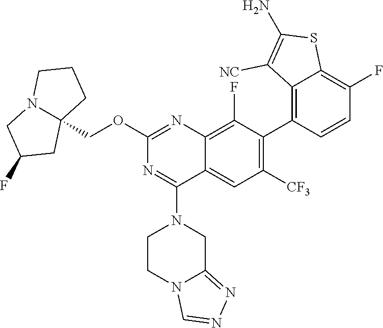 [SMILES: N#Cc1c(N)sc2c(F)ccc(-c3c(C(F)(F)F)cc4c(N5CCn6cnnc6C5)nc(OC[C@@]56CCCN5C[C@H](F)C6)nc4c3F)c12] | LCMS m/z [M + 1]: 686.0 1H NMR (400 MHz, CD3OD) δ 8.57 (d, J = 16.8 Hz, 1H), 8.28 (d, J = 16 Hz, 1H), 7.36-7.23 (m, 1H), 7.07- 7.00 (m, 1H), 5.40-5.27 (m, 3H), 4.61 (s, 1H), 4.50- 4.34 (m, 5H), 3.07 (s, 3H), 2.41-2.16 (m, 3H), 2.03- 1.95 (m, 3H). |
| 294a | 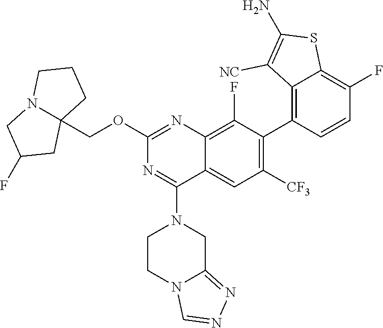 [SMILES: N#Cc1c(N)sc2c(F)ccc(-c3c(C(F)(F)F)cc4c(N5CCn6cnnc6C5)nc(OCC56CCCN5CC(F)C6)nc4c3F)c12] | |
| 295 | 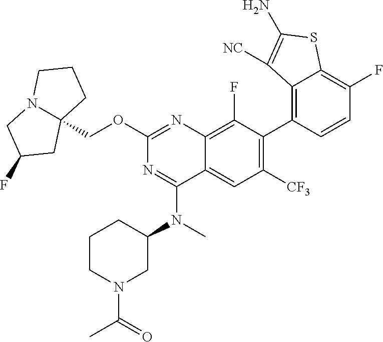 [SMILES: CC(=O)N1CCC[C@@H](N(C)c2nc(OC[C@@]34CCCN3C[C@H](F)C4)nc3c(F)c(-c4ccc(F)c5sc(N)c(C#N)c45)c(C(F)(F)F)cc23)C1] | LCMS m/z [M + 1]: 718.1 1H NMR (400 MHz, CD3OD) δ 8.32-8.28 (m, 1H), 7.25-7.15 (m, 1H), 7.00 (t, 1H), 5.41-5.34 (d, 0.5H), 5.27-5.20 (d, 0.5H), 4.76-4.51 (m, 2H), 4.50-4.14 (m, 3H), 4.03-4.87 (d, 1H), 3.58-3.38 (m, 3H), 3.26-2.95 (m, 4H), 2.94-2.80 (m, 1H), 2.46-1.47 (m, 13H). |
| 295a | 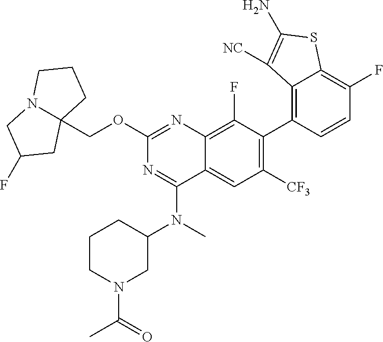 [SMILES: CC(=O)N1CCCC(N(C)c2nc(OCC34CCCN3CC(F)C4)nc3c(F)c(-c4ccc(F)c5sc(N)c(C#N)c45)c(C(F)(F)F)cc23)C1] | |
| 296 | 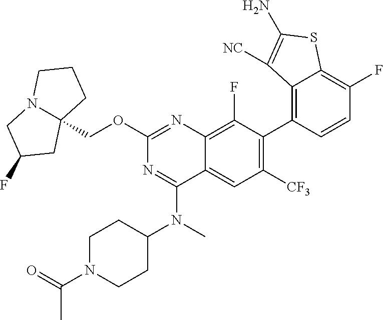 [SMILES: CC(=O)N1CCC(N(C)c2nc(OC[C@@]34CCCN3C[C@H](F)C4)nc3c(F)c(-c4ccc(F)c5sc(N)c(C#N)c45)c(C(F)(F)F)cc23)CC1] | LCMS m/z [M + 1]: 718.1 1H NMR (400 MHz, CD3OD) δ 8.28 (s, 1H), 7.23- 7.19 (m, 1H), 7.03-6.98 (t, 1H), 5.38 (s, 0.5H), 5.25 (s, 0.5H), 4.74 (d, 1H), 4.34-4.23 (m, 2H), 4.10 (d, 1H), 3.38 (s, 3H), 3.37-3.31 (m, 2H), 3.29-3.22 (m, 3H), 3.08-3.01 (m, 1H), 2.82-2.74 (t, 1H), 2.39-2.32 (m, 2H), 2.20-2.12 (m, 4H), 2.08-1.96 (m, 5H), 1.94- 1.86 (m, 2H). |
| 296a | 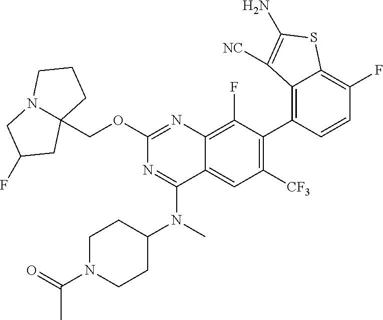 [SMILES: CC(=O)N1CCC(N(C)c2nc(OCC34CCCN3CC(F)C4)nc3c(F)c(-c4ccc(F)c5sc(N)c(C#N)c45)c(C(F)(F)F)cc23)CC1] | |
| 297 | 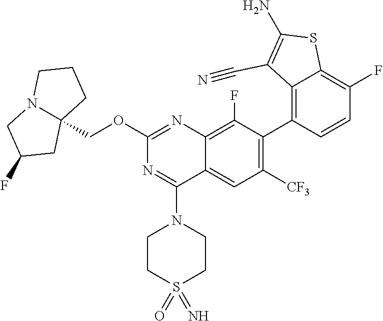 [SMILES: N#Cc1c(N)sc2c(F)ccc(-c3c(C(F)(F)F)cc4c(N5CCS(=N)(=O)CC5)nc(OC[C@@]56CCCN5C[C@H](F)C6)nc4c3F)c12] | LCMS m/z [M + 1]: 696.0 1H NMR (400 MHz, DMSO_d6 & D2O) δ 8.15 (s, 1H), 7.25 (t, 1H), 7.15 (t, 1H), 5.39 (s, 0.5H), 5.26 (s, 0.5H), 4.30-4.05 (m, 6H), 3.41-3.13 (m, 7H), 2.94- 2.85 (m, 1H), 2.24-2.06 (m, 3H), 1.95-1.75 (m, 3H). |
| 297a | 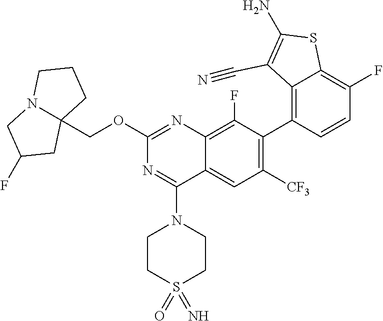 [SMILES: N#Cc1c(N)sc2c(F)ccc(-c3c(C(F)(F)F)cc4c(N5CCS(=N)(=O)CC5)nc(OCC56CCCN5CC(F)C6)nc4c3F)c12] | |
| 298 | 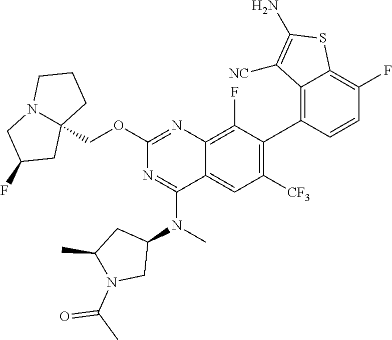 [SMILES: CC(=O)N1C[C@H](N(C)c2nc(OC[C@@]34CCCN3C[C@H](F)C4)nc3c(F)c(-c4ccc(F)c5sc(N)c(C#N)c45)c(C(F)(F)F)cc23)C[C@@H]1C] | LCMS m/z [M + 1]: 718.1 1H NMR (400 MHz, CD3OD) δ 8.29 (s, 1H), 7.23- 7.20 (m, 1H), 7.03-6.99 (m, 1H), 5.37 (s, 0.5H), 5.24 (s, 0.5H), 5.12-5.08 (m, 1H), 4.35-4.30 (m, 1H), 4.26-4.06 (m, 3H), 3.70-3.59 (m, 1H), 3.46 (s, 3H), 3.22 (s, 2H), 3.05-3.00 (m, 1H), 2.66-2.61 (m, 1H), 2.34-1.87 (m, 11H), 1.47-1.41 (m, 3H). |
| 298a | 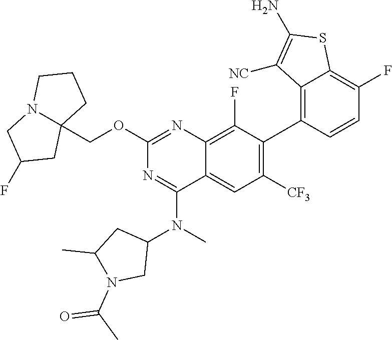 [SMILES: CC(=O)N1CC(N(C)c2nc(OCC34CCCN3CC(F)C4)nc3c(F)c(-c4ccc(F)c5sc(N)c(C#N)c45)c(C(F)(F)F)cc23)CC1C] | |
| 299 | 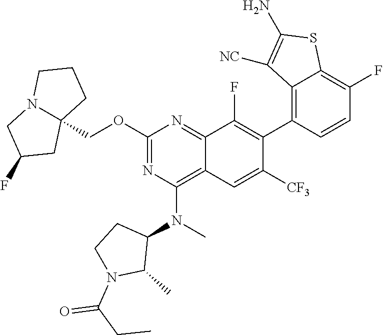 [SMILES: CCC(=O)N1CC[C@@H](N(C)c2nc(OC[C@@]34CCCN3C[C@H](F)C4)nc3c(F)c(-c4ccc(F)c5sc(N)c(C#N)c45)c(C(F)(F)F)cc23)[C@@H]1C] | LCMS m/z [M + 1]: 732.2 1H NMR (400 MHz, CD3OD) δ 8.36 (s, 1H), 7.21- 7.25 (m, 1H), 7.03 (t, J = 8.88 Hz, 1H), 5.56 (d, J = 51.72 Hz, 1H), 4.93-5.04 (m, 2H), 4.60-4.74 (m, 2H), 3.47-4.41 (m, 9H), 2.12-2.76 (m, 10H), 1.37-1.46 (m, 3H), 1.12-1.16 (m, 3H). |
| 299a | 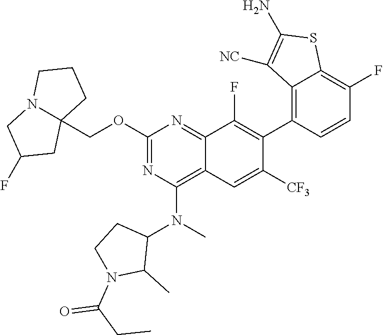 [SMILES: CCC(=O)N1CCC(N(C)c2nc(OCC34CCCN3CC(F)C4)nc3c(F)c(-c4ccc(F)c5sc(N)c(C#N)c45)c(C(F)(F)F)cc23)C1C] | |
| 300 | 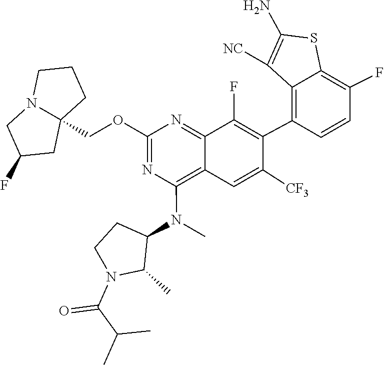 [SMILES: CC(C)C(=O)N1CC[C@@H](N(C)c2nc(OC[C@@]34CCCN3C[C@H](F)C4)nc3c(F)c(-c4ccc(F)c5sc(N)c(C#N)c45)c(C(F)(F)F)cc23)[C@@H]1C] | LCMS m/z [M + 1]: 746.2 1H NMR (400 MHz, CD3OD) δ 8.36 (s, 1H), 7.21- 7.25 (m, 1H), 7.03 (t, J = 8.88 Hz, 1H), 5.56 (d, J = 51.72 Hz, 1H), 5.05-5.10 (m, 2H), 4.61-4.75 (m, 2H), 3.45-4.43 (m, 9H), 2.11-2.87 (m, 9H), 1.36-1.48 (m, 3H), 1.12-1.16 (m, 6H). |
| 300a | 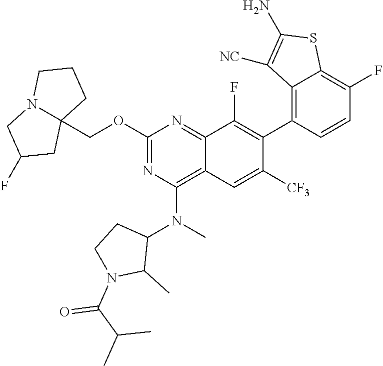 [SMILES: CC(C)C(=O)N1CCC(N(C)c2nc(OCC34CCCN3CC(F)C4)nc3c(F)c(-c4ccc(F)c5sc(N)c(C#N)c45)c(C(F)(F)F)cc23)C1C] | |
| 301 | 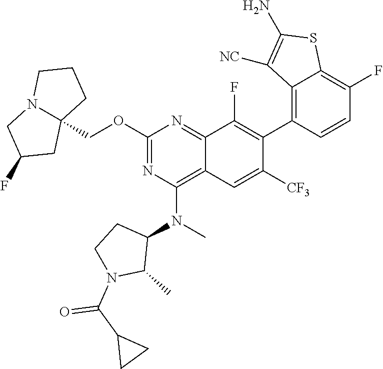 [SMILES: C[C@H]1[C@H](N(C)c2nc(OC[C@@]34CCCN3C[C@H](F)C4)nc3c(F)c(-c4ccc(F)c5sc(N)c(C#N)c45)c(C(F)(F)F)cc23)CCN1C(=O)C1CC1] | LCMS m/z [M + 1]: 744.2 1H NMR (400 MHz, CD3OD) δ 8.36 (s, 1H), 7.21- 7.25 (m, 1H), 7.03 (t, J = 8.88 Hz, 1H), 5.56 (d, J = 51.72 Hz, 1H), 5.05-5.14 (m, 2H), 4.61-4.75 (m, 2H), 3.47-4.41 (m, 10H), 1.83-2.71 (m, 9H), 1.54-1.56 (m, 1H), 1.36-1.40 (m, 2H), 0.81-1.02 (m, 4H). |
| 301a | 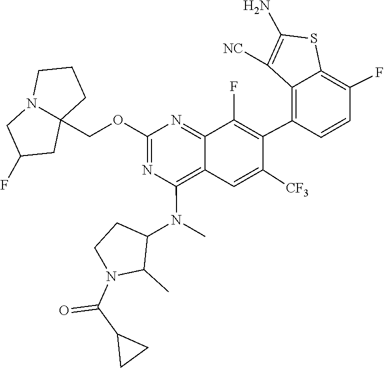 [SMILES: CC1C(N(C)c2nc(OCC34CCCN3CC(F)C4)nc3c(F)c(-c4ccc(F)c5sc(N)c(C#N)c45)c(C(F)(F)F)cc23)CCN1C(=O)C1CC1] | |
| 302 | 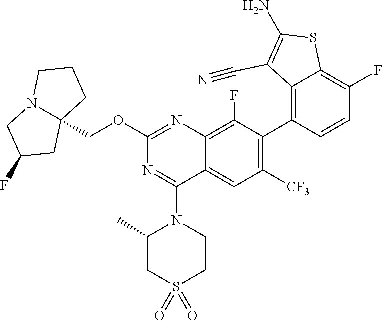 [SMILES: C[C@H]1CS(=O)(=O)CCN1c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 711.0 1H NMR (400 MHz, CD3OD) δ 8.07 (d, J = 7.6 Hz, 1H), 7.24-7.19 (m, 1H), 7.04-6.99 (m, 1H), 5.44 (d, J = 5.4 Hz, 0.51H), 5.31 (s, 0.59H), 5.17-5.13 (m, 1H), 4.69 (d, J = 15.6 Hz, 1H), 4.48-4.34 (m, 2H), 4.19- 4.11 (m, 1H), 3.69-3.36 (m, 5H), 3.25-3.11 (m, 3H), 2.47-1.82 (m, 9H). |
| 302a | 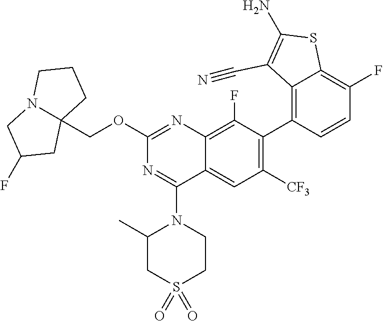 [SMILES: CC1CS(=O)(=O)CCN1c1nc(OCC23CCCN2CC(F)C3)nc2c(F)c(-c3ccc(F)c4sc(N)c(C#N)c34)c(C(F)(F)F)cc12] | |
| 303 | 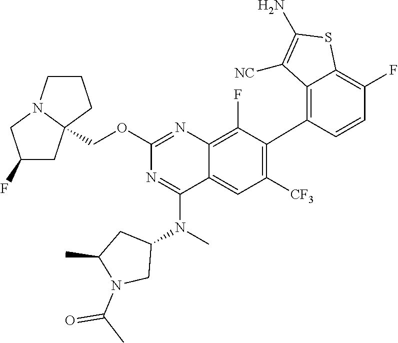 [SMILES: CC(=O)N1C[C@@H](N(C)c2nc(OC[C@@]34CCCN3C[C@H](F)C4)nc3c(F)c(-c4ccc(F)c5sc(N)c(C#N)c45)c(C(F)(F)F)cc23)C[C@@H]1C] | LCMS m/z [M + 1]: 718.2 1H NMR (400 MHz, CD3OD) δ 8.44-8.34 (m, 1H), 7.28-7.19 (m, 1H), 7.01 (t, J = 8.8 Hz, 1H), 5.61 (s, 0.5H), 5.48 (s, 0.5H) 4.75-4.60 (m, 2H), 4.11-3.78 (m, 5H), 3.75-3.62 (m, 1H), 3.54-3.39 (m, 4H), 3.25- 3.16 (m, 2H), 2.75-2.51 (m, 3H), 2.47-2.28 (m, 3H), 2.23-2.02 (m, 4H), 1.22-1.08 (m, 3H). |
| 303a | 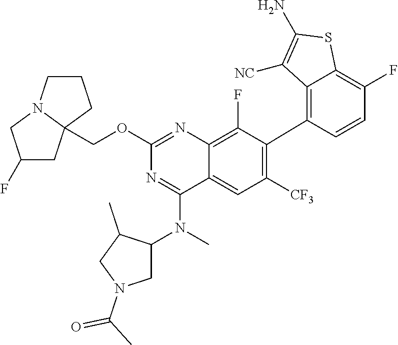 [SMILES: CC(=O)N1CC(C)C(N(C)c2nc(OCC34CCCN3CC(F)C4)nc3c(F)c(-c4ccc(F)c5sc(N)c(C#N)c45)c(C(F)(F)F)cc23)C1] | |
| 304 | 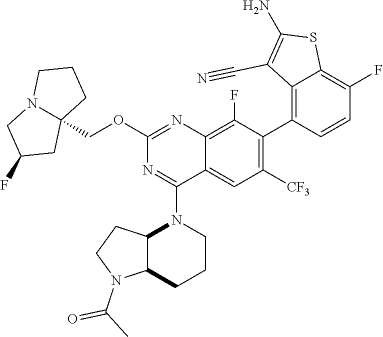 | LCMS m/z [M + 1]: 730.1 1H NMR (400 MHz, CD3OD) δ 8.12 (s, 1H), 7.27- 7.17 (m, 1H), 7.01 (t, J = 8.8 Hz, 1H), 5.40 (s, 0.5H), 5.27 (s, 0.5H), 5.18-5.07 (m, 1H), 4.42-4.03 (m, 4H), 3.87-3.50 (m, 4H), 3.43-3.35 (m, 1H), 3.08 (s, 1H), 2.74-2.53 (m, 1H), 2.46-1.51 (m, 15H). |
| 304a | 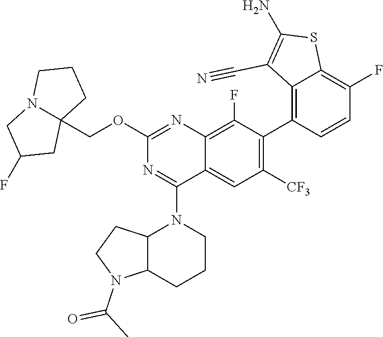 [SMILES: CC(=O)N1CCC2C1CCCN2c1nc(OCC23CCCN2CC(F)C3)nc2c(F)c(-c3ccc(F)c4sc(N)c(C#N)c34)c(C(F)(F)F)cc12] | |
| 305 | 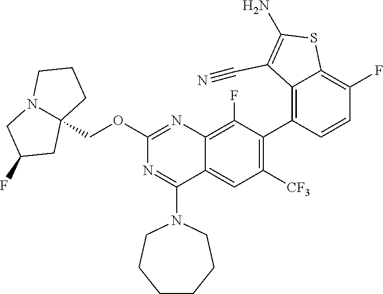 [SMILES: N#Cc1c(N)sc2c(F)ccc(-c3c(C(F)(F)F)cc4c(N5CCCCCC5)nc(OC[C@@]56CCCN5C[C@H](F)C6)nc4c3F)c12] | LCMS m/z [M + 1]: 661.1 1H NMR (400 MHz, CD3OD) δ 8.308 (s, 1H), 7.23- 7.18 (m, 1H), 7.004 (t, 1H), 5.420 (s, 0.5H), 5.286 (s, 0.5H), 4.42-4.35 (m, 1H), 4.33-4.27 (m, 1H), 4.061 (t, 4H), 3.51-3.33 (m, 3H), 3.14-3.07 (m, 1H), 2.44- 2.18 (m, 3H), 2.10-1.86 (m, 7H), 1.717 (s, 4H). |
| 305a | 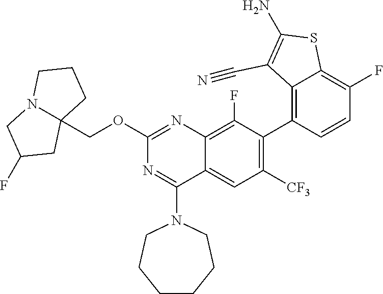 [SMILES: N#Cc1c(N)sc2c(F)ccc(-c3c(C(F)(F)F)cc4c(N5CCCCCC5)nc(OCC56CCCN5CC(F)C6)nc4c3F)c12] | |
| 306 | 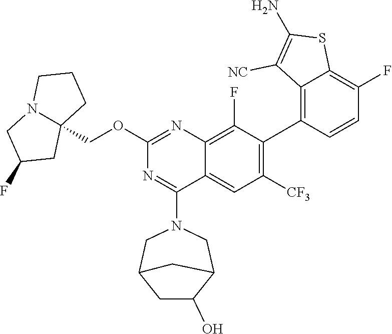 [SMILES: N#Cc1c(N)sc2c(F)ccc(-c3c(C(F)(F)F)cc4c(N5CC6CC(O)C(C6)C5)nc(OC[C@@]56CCCN5C[C@H](F)C6)nc4c3F)c12] | LCMS m/z [M + 1]: 689.3 1H NMR (400 MHz, CD3OD) δ 8.15 (s, 1H), 7.24- 7.17 (m, 1H), 7.00 (t, J = 9.2 Hz, 1H), 5.42 (s, 0.5H), 5.32 (s, 0.5H), 4.79-4.62 (m, 1H), 4.53-4.25 (m, 3H), 4.22-4.14 (m, 1H), 3.64-3.42 (m, 3H), 3.40-3.33 (m, 2H), 3.14-3.05 (m, 1H), 2.57-2.37 (m, 2H), 2.35-2.17 (m, 3H), 2.11-1.88 (m, 5H), 1.78 (d, J = 11.2 Hz, 1H), 1.61-1.53 (m, 1H). |
| 306a | 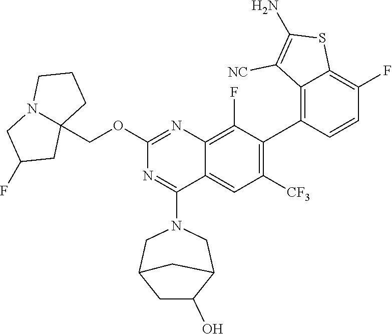 [SMILES: N#Cc1c(N)sc2c(F)ccc(-c3c(C(F)(F)F)cc4c(N5CC6CC(O)C(C6)C5)nc(OCC56CCCN5CC(F)C6)nc4c3F)c12] | |
| 307 | 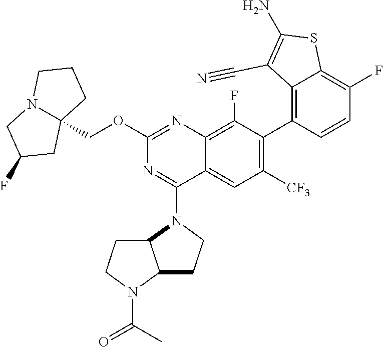 | LCMS m/z [M + 1]: 716.22 1H NMR (400 MHz, CD3OD) δ 8.43-8.33 (m, 1H), 7.24-7.15 (m, 1H), 7.04-6.96 (m, 1H), 5.45-5.22 (m, 2H), 4.73-4.61 (m, 1H), 4.48-3.95 (m, 5H), 3.89-3.73 (m, 1H), 3.66-3.52 (m, 1H), 3.49-3.33 (m, 2H), 3.08 (s, 1H), 2.69-2.53 (m, 1H), 2.45-2.12 (m, 9H), 2.10- 1.86 (m, 3H). |
| 307a | 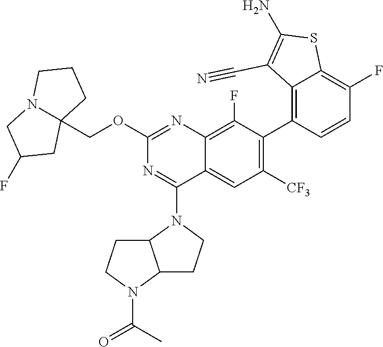 [SMILES: CC(=O)N1CCC2C1CCN2c1nc(OCC23CCCN2CC(F)C3)nc2c(F)c(-c3ccc(F)c4sc(N)c(C#N)c34)c(C(F)(F)F)cc12] | |
| 308 | 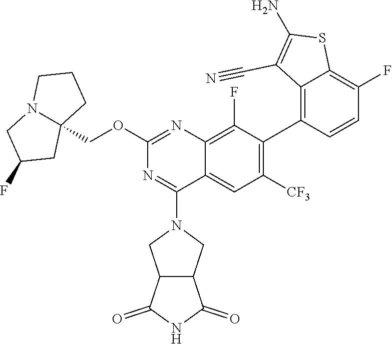 [SMILES: N#Cc1c(N)sc2c(F)ccc(-c3c(C(F)(F)F)cc4c(N5CC6C(=O)NC(=O)C6C5)nc(OC[C@@]56CCCN5C[C@H](F)C6)nc4c3F)c12] | LCMS m/z [M + 1]: 702.16 1H NMR (400 MHz, CD3OD) δ 8.37 (s, 1H), 7.28- 7.16 (m, 1H), 7.08-6.94 (m, 1H), 5.37 (s, 0.5H), 5.38-5.20 (m, 1.5H), 4.70-4.60 (m, 1H), 4.42-4.24 (m, 2H), 3.76-3.67 (m, 1H), 3.67-3.33 (m, 4H), 3.17- 3.07 (m, 1H), 2.49-1.88 (m, 8H). |
| 308a | 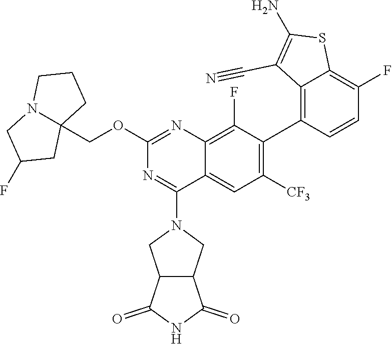 [SMILES: N#Cc1c(N)sc2c(F)ccc(-c3c(C(F)(F)F)cc4c(N5CC6C(=O)NC(=O)C6C5)nc(OCC56CCCN5CC(F)C6)nc4c3F)c12] | |
| 309 | 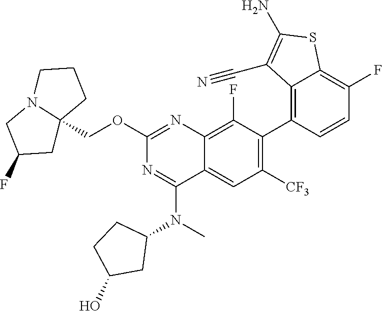 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@H]1CC[C@@H](O)C1] | LCMS m/z [M + 1]: 677.0 1H NMR (400 MHz, CD3OD) δ 8.29 (s, 1H), 7.17- 7.24 (m, 1H), 7.01 (t, 1H), 5.41 (s, 0.5H), 5.27 (s, 0.5H), 5.10-5.19 (m, 1H), 4.23-4.43 (m, 3H), 3.46 (s, 3H), 3.36 (s, 3H), 3.06-3.12 (m, 1H), 2.26-2.49 (m, 3H), 1.98-2.24 (m, 5H), 1.93 (s, 1H), 1.71-1.87 (m, 3H). |
| 309a | 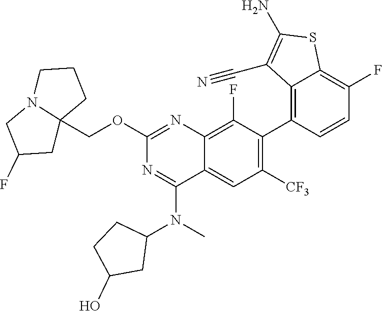 [SMILES: CN(c1nc(OCC23CCCN2CC(F)C3)nc2c(F)c(-c3ccc(F)c4sc(N)c(C#N)c34)c(C(F)(F)F)cc12)C1CCC(O)C1] | |
| 310 | 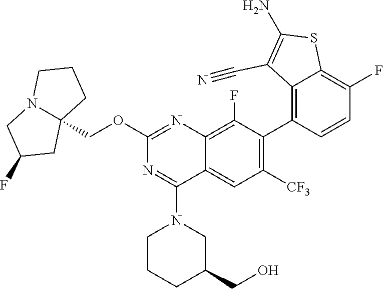 [SMILES: N#Cc1c(N)sc2c(F)ccc(-c3c(C(F)(F)F)cc4c(N5CCC[C@H](CO)C5)nc(OC[C@@]56CCCN5C[C@H](F)C6)nc4c3F)c12] | LCMS m/z [M + 1]: 677.1 1H NMR (400 MHz, CD3OD) δ 8.19 (s, 1H), 7.19- 7.23 (m, 1H), 6.99-7.03 (m, 1H), 5.50 (s, 0.5H), 5.37 (s, 0.5H), 4.57-4.62 (m, 1H), 4.42-4.55 (m, 3H), 3.69-3.74 (m, 1H), 3.55-3.60 (m, 3H), 3.41-3.50 (m, 2H), 3.19-3.27 (m, 2H), 2.40-2.56 (m, 2H), 2.26-2.32 (m, 1H), 2.13-2.19 (m, 2H), 1.93-2.02 (m, 4H), 1.79 (s, 1H) 1.42-1.50 (m, 1H). |
| 310a | 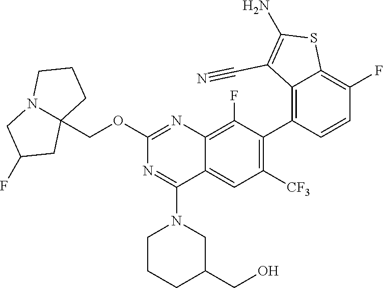 [SMILES: N#Cc1c(N)sc2c(F)ccc(-c3c(C(F)(F)F)cc4c(N5CCCC(CO)C5)nc(OCC56CCCN5CC(F)C6)nc4c3F)c12] | |
| 311 | 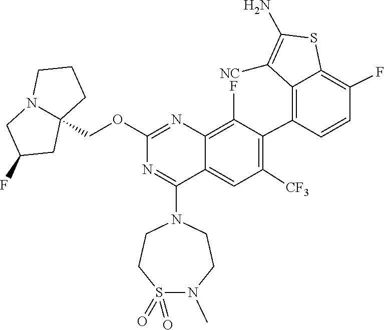 [SMILES: CN1CCN(c2nc(OC[C@@]34CCCN3C[C@H](F)C4)nc3c(F)c(-c4ccc(F)c5sc(N)c(C#N)c45)c(C(F)(F)F)cc23)CCS1(=O)=O] | LCMS m/z [M + 1]: 726.0 1H NMR (400 MHz, CD3OD) δ 8.28 (s, 1H), 7.25 (t, 1H), 7.02 (t, 1H), 5.40 (s, 0.5H), 5.27 (s, 0.5H), 4.22- 4.27 (m, 6H), 3.80-3.35 (m, 7H), 3.15-3.04 (m, 4H), 2.42-2.15 (m, 3H), 2.06-1.91 (m, 3H). |
| 311a | 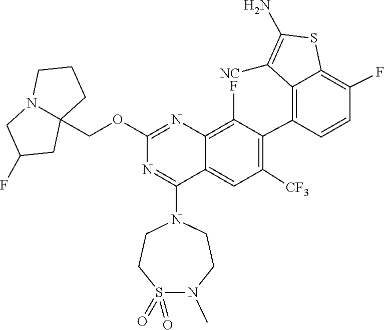 [SMILES: CN1CCN(c2nc(OCC34CCCN3CC(F)C4)nc3c(F)c(-c4ccc(F)c5sc(N)c(C#N)c45)c(C(F)(F)F)cc23)CCS1(=O)=O] | |
| 312 | 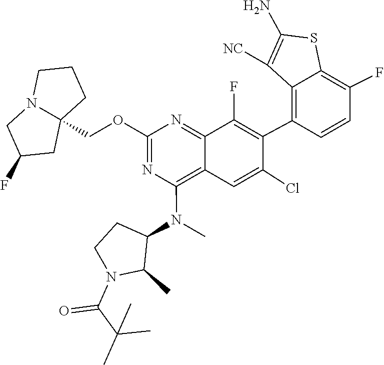 [SMILES: C[C@@H]1[C@H](N(C)c2nc(OC[C@@]34CCCN3C[C@H](F)C4)nc3c(F)c(-c4ccc(F)c5sc(N)c(C#N)c45)c(Cl)cc23)CCN1C(=O)C(C)(C)C] | LCMS m/z [M + 1]: 760.2 1H NMR (400 MHz, CD3OD) δ 8.41 (s, 1H), 7.31- 7.18 (m, 1H), 7.03 (t, 1H), 5.34-5.62 (m, 1H), 5.13- 5.04 (m, 1H), 4.61-4.78 (m, 3H), 3.71-4.05 (m, 5H), 3.62 (s, 3H), 3.44-3.50 (m, 1H), 2.55-2.74 (m, 3H), 2.32-2.40 (m, 4H), 2.14-2.17 (m, 1H), 1.31 (s, 9H), 1.05-1.07 (m, 3H). |
| 312a | 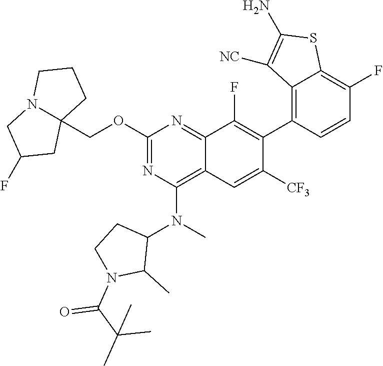 [SMILES: CC1C(N(C)c2nc(OCC34CCCN3CC(F)C4)nc3c(F)c(-c4ccc(F)c5sc(N)c(C#N)c45)c(C(F)(F)F)cc23)CCN1C(=O)C(C)(C)C] | |
| 313 | 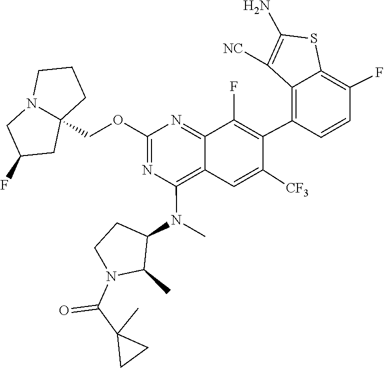 [SMILES: C[C@@H]1[C@H](N(C)c2nc(OC[C@@]34CCCN3C[C@H](F)C4)nc3c(F)c(-c4ccc(F)c5sc(N)c(C#N)c45)c(C(F)(F)F)cc23)CCN1C(=O)C1(C)CC1] | LCMS m/z [M + 1]: 758.3 1H NMR (400 MHz, CD3OD) δ 8.41 (s, 1H), 7.22- 7.25 (m, 1H), 7.01-7.05 (t, 1H), 5.49-5.62 (m, 1H), 4.99 (s, 1H), 4.76-4.84 (m, 3H), 3.78-3.75 (m, 5H), 3.65 (s, 3H), 3.46-3.47 (m, 1H), 2.54-2.73 (m, 3H), 2.31-2.47 (m, 4H), 2.13-2.15 (m, 1H), 1.37 (s, 3H), 0.93-1.08 (m , 5H), 0.62-0.68 (m, 2H). |
| 313a | 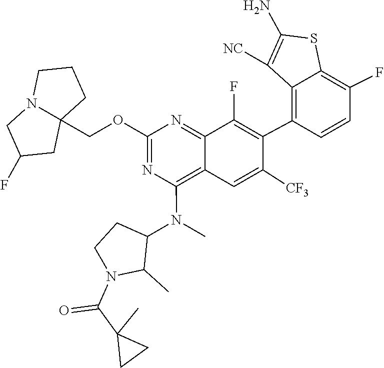 [SMILES: CC1C(N(C)c2nc(OCC34CCCN3CC(F)C4)nc3c(F)c(-c4ccc(F)c5sc(N)c(C#N)c45)c(C(F)(F)F)cc23)CCN1C(=O)C1(C)CC1] | |
| 314 | 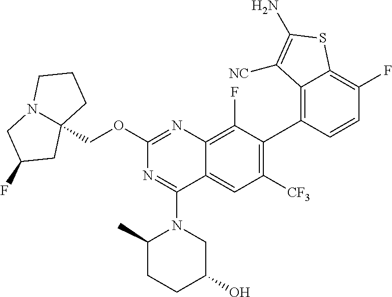 [SMILES: C[C@@H]1CC[C@@H](O)CN1c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 677.1 1H NMR (400 MHz, CD3OD) δ 8.62 (d, 1H), 7.27- 7.15 (m, 1H), 7.06-6.91 (m, 1H), 5.38 (s, 0.5H), 5.25 (s, 0.5H), 4.96 (s, 1H), 4.38-4.23 (m, 3H), 4.09 (s, 1H), 3.71 (d, 1H), 3.36-3.32 (m, 1H), 3.27-3.22 (m, 2H), 3.04 (s, 1H), 2.42-2.20 (m, 3H), 2.20-2.08 (m, 2H), 2.05-1.95 (m, 2H), 1.95-1.85 (m, 1H), 1.80-1.68 (m, 1H), 1.55-1.48 (m, 1H), 1.47-1.42 (m, 3H). |
| 314a | 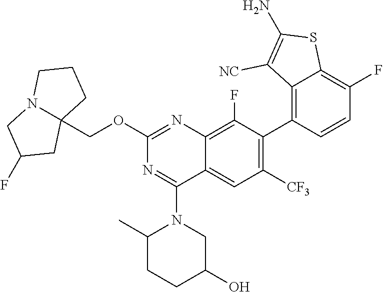 [SMILES: CC1CCC(O)CN1c1nc(OCC23CCCN2CC(F)C3)nc2c(F)c(-c3ccc(F)c4sc(N)c(C#N)c34)c(C(F)(F)F)cc12] | |
| 315 | 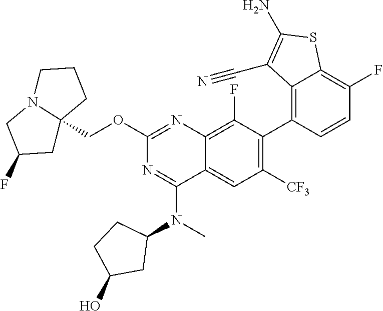 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@@H]1CC[C@H](O)C1] | LCMS m/z [M + 1]: 677.0 1H NMR (400 MHz, CD3OD) δ 8.28 (s, 1H), 7.19- 7.22 (m, 1H), 6.98-7.03 (m, 1H), 5.39 (s, 0.5H), 5.25 (s, 0.5H), 5.11-5.16 (m, 1H), 4.24-4.33 (m, 3H), 3.41-3.47 (m, 3H), 3.33-3.38 (m, 3H), 3.05 (s, 1H), 2.27-2.43 (m, 3H), 2.16-2.20 (m, 2H), 2.01-2.03 (m, 3H), 1.83-1.91 (m, 4H). |
| 315a | 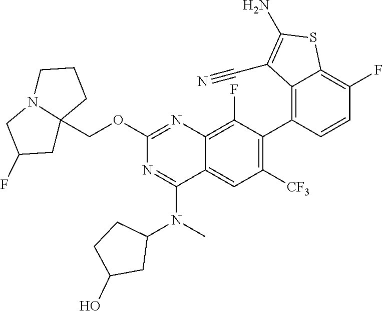 [SMILES: CN(c1nc(OCC23CCCN2CC(F)C3)nc2c(F)c(-c3ccc(F)c4sc(N)c(C#N)c34)c(C(F)(F)F)cc12)C1CCC(O)C1] | |
| 316 | 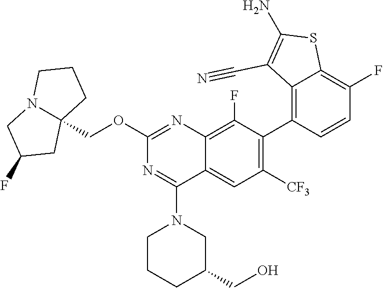 [SMILES: N#Cc1c(N)sc2c(F)ccc(-c3c(C(F)(F)F)cc4c(N5CCC[C@@H](CO)C5)nc(OC[C@@]56CCCN5C[C@H](F)C6)nc4c3F)c12] | LCMS m/z [M + 1]: 677.0 1H NMR (400 MHz, CD3OD) δ 8.15 (s, 1H), 7.19- 7.22 (m, 1H), 7.00 (t, 1H), 5.36 (s, 0.5H), 5.23 (s, 0.5H), 4.55 (t, 1H), 4.37-4.66 (m, 1H), 4.30-4.33 (m, 1H), 4.21-4.25 (m, 1H), 3.54-3.59 (m, 1H), 3.44- 3.501 (m, 1H), 3.58-3.41(m, 1H), 3.12-3.23 (m, 4H), 2.97-3.03 (m, 1H), 2.11-2.38 (m, 3H), 1.77-2.00 (m, 7H), 1.42-1.51 (m, 1H). |
| 316a | 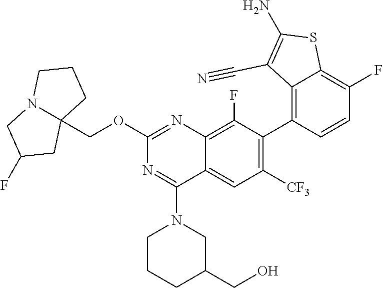 [SMILES: N#Cc1c(N)sc2c(F)ccc(-c3c(C(F)(F)F)cc4c(N5CCCC(CO)C5)nc(OCC56CCCN5CC(F)C6)nc4c3F)c12] | |
| 317 | 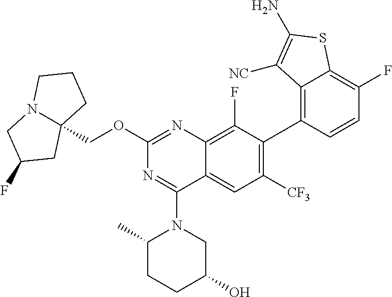 [SMILES: C[C@H]1CC[C@@H](O)CN1c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 677.3 1H NMR (400 MHz, CD3OD) δ 8.09 (s, 1H), 7.20- 7.23 (m, 1H), 7.01 (t, 1H), 5.46 (s, 0.5H), 5.34 (s, 0.5H), 4.79-4.82 (m, 1H), 4.44-4.50 (m, 2H), 4.34- 4.38 (m, 1H), 3.84-3.88 (m, 1H), 3.42-3.58 (m, 3H), 3.12-3.25 (m, 2H), 2.38-2.51 (m, 1H), 2.23-2.28 (m, 1H), 1.98-2.13 (m, 5H), 1.75-1.84 (m, 2H), 1.58-1.60 (m, 1H), 1.54-1.56 (m, 3H). |
| 319 | 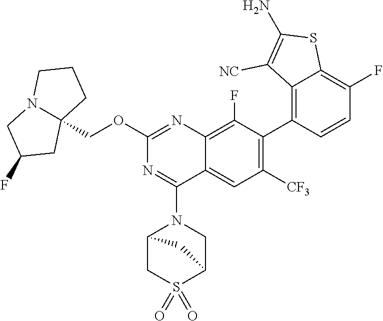 [SMILES: N#Cc1c(N)sc2c(F)ccc(-c3c(C(F)(F)F)cc4c(N5C[C@@H]6C[C@H]5CS6(=O)=O)nc(OC[C@@]56CCCN5C[C@H](F)C6)nc4c3F)c12] | LCMS m/z [M + 1]: 709.0 1H NMR (400 MHz, CD3OD) δ 8.34-8.28 (m, 1H), 7.24-7.17 (m, 1H), 7.04-6.97 (m, 1H), 5.53 (s, 1H), 5.38 (s, 0.5H), 5.24 (s, 0.5H), 4.63-4.55 (m, 1H), 4.52-4.46 (m, 1H), 4.34-4.22 (m, 2H), 4.05 (s, 1H), 3.69-3.62 (m, 1H), 3.46-3.37 (m, 1H), 3.26-3.23 (m, 1H), 3.08-3.00 (m, 1H), 2.75-2.64 (m, 2H), 2.42-1.79 (m, 8H). |
| 319a | 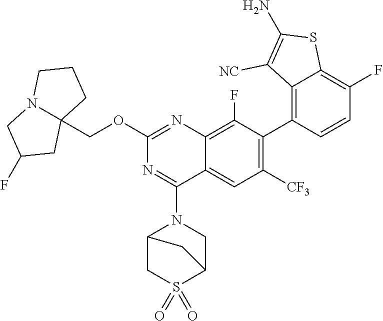 [SMILES: N#Cc1c(N)sc2c(F)ccc(-c3c(C(F)(F)F)cc4c(N5CC6CC5CS6(=O)=O)nc(OCC56CCCN5CC(F)C6)nc4c3F)c12] | |
| 320 | 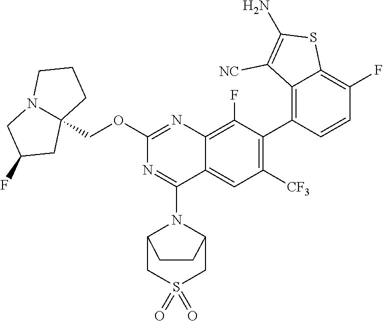 [SMILES: N#Cc1c(N)sc2c(F)ccc(-c3c(C(F)(F)F)cc4c(N5C6CCC5CS(=O)(=O)C6)nc(OC[C@@]56CCCN5C[C@H](F)C6)nc4c3F)c12] | LCMS m/z [M + 1]: 723.0 1H NMR (400 MHz, CD3OD) δ 8.18 (s, 1H), 7.25- 7.19 (m, 1H), 7.05-6.98 (m, 1H), 5.52-5.40 (m, 2H), 5.37 (s, 0.5H), 5.24 (s, 0.5H), 4.37-4.23 (m, 2H), 3.90-3.76 (m, 2H), 3.50-3.40 (m, 2H), 3.34-3.32 (m, 1H), 3.27-3.20 (m, 2H), 3.08-2.98 (m, 1H), 2.62-2.51 (m, 2H), 2.38-2.11 (m, 5H), 2.04-1.85 (m, 3H). |
| 320a | 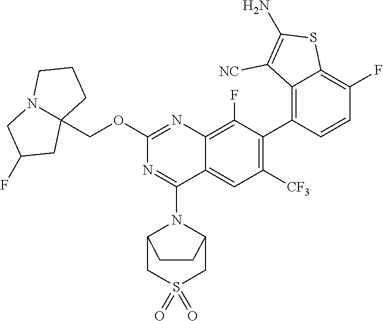 [SMILES: N#Cc1c(N)sc2c(F)ccc(-c3c(C(F)(F)F)cc4c(N5C6CCC5CS(=O)(=O)C6)nc(OCC56CCCN5CC(F)C6)nc4c3F)c12] | |
| 321 | 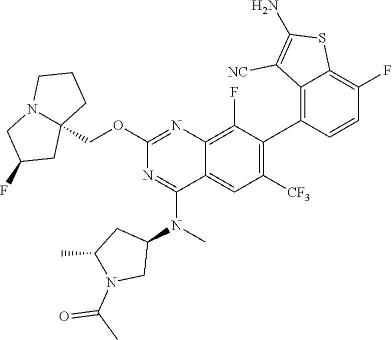 [SMILES: CC(=O)N1C[C@H](N(C)c2nc(OC[C@@]34CCCN3C[C@H](F)C4)nc3c(F)c(-c4ccc(F)c5sc(N)c(C#N)c45)c(C(F)(F)F)cc23)C[C@H]1C] | LCMS m/z [M + 1]: 718.3 1H NMR (400 MHz, CD3OD) δ 8.31 (m, 1H), 7.24- 7.21 (m, 1H), 7.04-7.00 (m, 1H), 5.49-5.48 (m, 1H), 5.40 (m, 0.5H), 5.27 (m, 0.5H), 4.43-4.28 (m, 3H), 4.11-4.02 (m, 1H), 3.95-3.86 (m, 1H), 3.74-3.65 (m, 1H), 3.44-3.41 (m, 3H), 3.08-3.06 (m, 1H), 2.67-2.49 (m, 2H), 2.42-1.91 (m, 11H), 1.44-1.30 (m, 3H). |
| 321a | 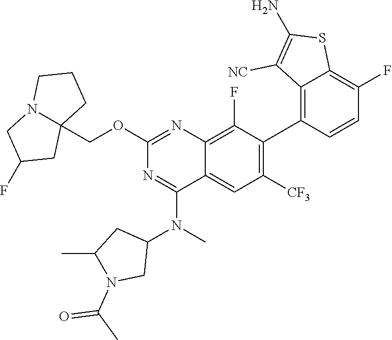 [SMILES: CC(=O)N1CC(N(C)c2nc(OCC34CCCN3CC(F)C4)nc3c(F)c(-c4ccc(F)c5sc(N)c(C#N)c45)c(C(F)(F)F)cc23)CC1C] | |
| 322 | 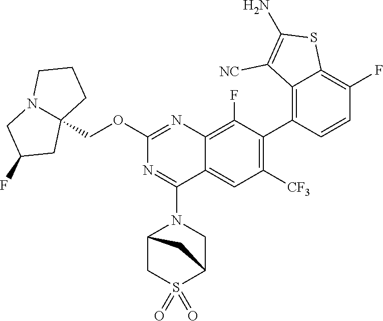 [SMILES: N#Cc1c(N)sc2c(F)ccc(-c3c(C(F)(F)F)cc4c(N5C[C@H]6C[C@@H]5CS6(=O)=O)nc(OC[C@@]56CCCN5C[C@H](F)C6)nc4c3F)c12] | LCMS m/z [M + 1]: 709.2 1H NMR (400 MHz, CD3OD) δ 8.54 (m, 1H), 7.25- 7.19 (m, 1H), 7.04-6.99 (m, 1H), 5.54 (m, 1H), 5.38 (s, 0.5H), 5.24 (s, 0.5H), 4.63-4.57 (m, 1H), 4.48- 4.43 (m, 1H), 4.33-4.28 (m, 1H), 4.26-4.22 (m, 1H), 4.06 (s, 1H), 3.67-3.64 (m, 1H), 3.48-3.41 (m, 1H), 3.27-3.23 (m, 2H), 3.07-3.00 (m, 1H), 2.76-2.65 (m, 2H), 2.40-1.88 (m, 7H). |
| 323 | 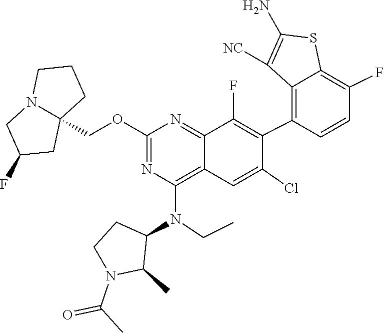 [SMILES: CCN(c1nc(OC[C@@]23CCCN2C[C@H](F)C3)nc2c(F)c(-c3ccc(F)c4sc(N)c(C#N)c34)c(Cl)cc12)[C@@H]1CCN(C(C)=O)[C@@H]1C] | LCMS m/z [M + 1]: 698.2 1H NMR (400 MHz, CD3OD) δ 8.10 (s, 1H), 7.22- 7.28 (m, 1H), 7.05 (t, J = 9.2 Hz, 1H), 5.57 (d, J = 51.6 Hz, 1H), 4.94-4.99 (m, 1H), 4.60-4.78 (m, 3H), 3.43-4.14 (m, 8H), 2.31-2.80 (m, 8H), 2.07- 2.11 (m, 3H), 1.26-1.37 (m, 3H), 0.93-1.06 (m, 3H). |
| 323a | 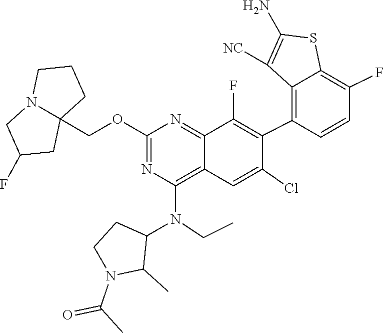 [SMILES: CCN(c1nc(OCC23CCCN2CC(F)C3)nc2c(F)c(-c3ccc(F)c4sc(N)c(C#N)c34)c(Cl)cc12)C1CCN(C(C)=O)C1C] | |
| 324 | 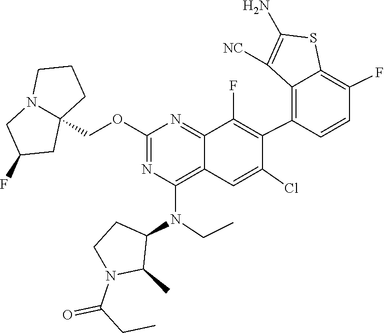 [SMILES: CCC(=O)N1CC[C@@H](N(CC)c2nc(OC[C@@]34CCCN3C[C@H](F)C4)nc3c(F)c(-c4ccc(F)c5sc(N)c(C#N)c45)c(Cl)cc23)[C@H]1C] | LCMS m/z [M + 1]: 712.3 1H NMR (400 MHz, CD3OD) δ 8.10 (s, 1H), 7.22- 7.26 (m, 1H), 7.05 (t, J = 9.2 Hz, 1H), 5.57 (d, J = 51.6 Hz, 1H), 4.94-4.99 (m, 1H), 4.60-4.80 (m, 2H), 3.43-4.14 (m, 9H), 2.31-2.77 (m, 9H), 2.14- 2.17 (m, 1H), 1.28-1.37 (m, 3H), 1.12-1.22 (m, 3H), 0.93-1.06 (m, 3H). |
| 324a | 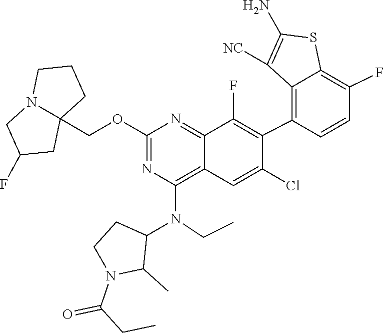 [SMILES: CCC(=O)N1CCC(N(CC)c2nc(OCC34CCCN3CC(F)C4)nc3c(F)c(-c4ccc(F)c5sc(N)c(C#N)c45)c(Cl)cc23)C1C] | |
| 325 | 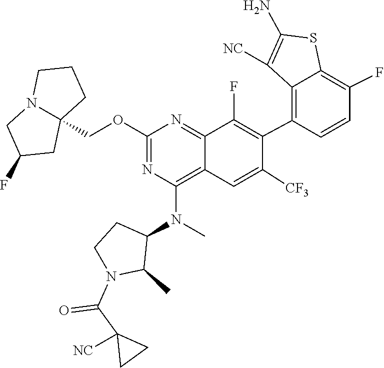 [SMILES: C[C@@H]1[C@H](N(C)c2nc(OC[C@@]34CCCN3C[C@H](F)C4)nc3c(F)c(-c4ccc(F)c5sc(N)c(C#N)c45)c(C(F)(F)F)cc23)CCN1C(=O)C1(C#N)CC1] | LCMS m/z [M + 1]: 769.2 1H NMR (400 MHz, CD3OD) δ 8.42 (s, 1H), 7.21- 7.25 (m, 1H), 7.03 (t, J = 9.28 Hz, 1H), 5.55 (d, J = 52.56 Hz, 1H), 4.60-5.02 (m, 3H), 3.86-4.27 (m, 4H), 3.67 (s, 3H), 3.43-3.48 (m, 1H), 2.16-2.73 (m, 8H), 1.10-1.64 (m, 7H). |
| 325a | 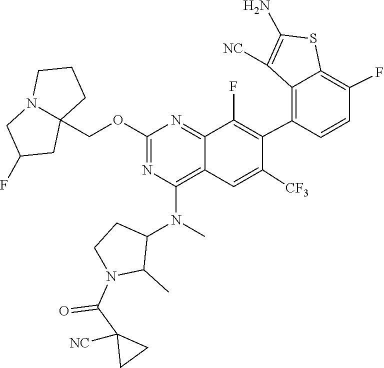 [SMILES: CC1C(N(C)c2nc(OCC34CCCN3CC(F)C4)nc3c(F)c(-c4ccc(F)c5sc(N)c(C#N)c45)c(C(F)(F)F)cc23)CCN1C(=O)C1(C#N)CC1] | |
| 326 | 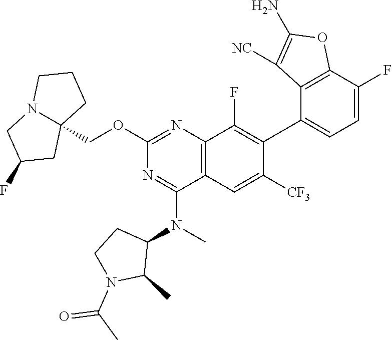 [SMILES: CC(=O)N1CC[C@@H](N(C)c2nc(OC[C@@]34CCCN3C[C@H](F)C4)nc3c(F)c(-c4ccc(F)c5oc(N)c(C#N)c45)c(C(F)(F)F)cc23)[C@H]1C] | LCMS m/z [M + 1]: 702.3 1H NMR (400 MHz, CD3OD) δ 8.42 (s, 1H), 7.10- 7.16 (m, 1H), 6.98-7.06 (m, 1H), 5.56 (d, J = 52.1 Hz, 1H), 4.96-5.04 (m, 1H), 4.59-4.83 (m, 3H), 3.80- 4.05 (m, 4H), 3.55-3.73 (m, 4H), 3.44-3.51 (m, 1H), 2.55-2.79 (m, 3H), 2.28-2.50 (m, 4H), 2.06-2.24 (m, 4H), 1.05-1.20 (m, 3H). |
| 326a | 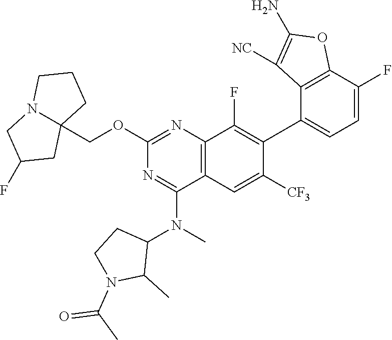 [SMILES: CC(=O)N1CCC(N(C)c2nc(OCC34CCCN3CC(F)C4)nc3c(F)c(-c4ccc(F)c5oc(N)c(C#N)c45)c(C(F)(F)F)cc23)C1C] | |
| 327 | 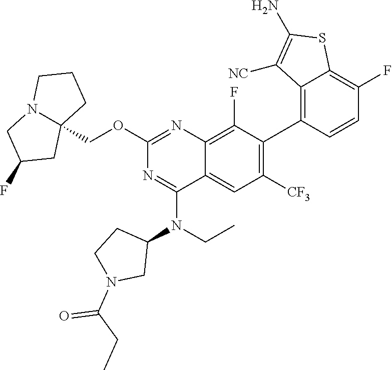 [SMILES: CCC(=O)N1CC[C@@H](N(CC)c2nc(OC[C@@]34CCCN3C[C@H](F)C4)nc3c(F)c(-c4ccc(F)c5sc(N)c(C#N)c45)c(C(F)(F)F)cc23)C1] | LCMS m/z [M + 1]: 732.2 1H NMR (400 MHz, CD3OD) δ 8.24 (s, 1H), 7.18- 7.25 (m, 1H), 7.01 (t, 1H), 5.37 (s, 0.5H), 5.23 (s, 0.5H), 4.97-5.09 (m, 1H), 4.19-4.37 (m, 2H), 3.69- 4.05 (m, 4H), 3.55-3.65 (m, 1H), 3.34-3.51 (m, 1H), 3.15-3.29 (m, 3H), 2.98-3.08 (m, 1H), 1.84-2.60 (m, 10H), 1.37-1.52 (m, 3H), 1.07-1.21 (m, 3H). |
| 327a | 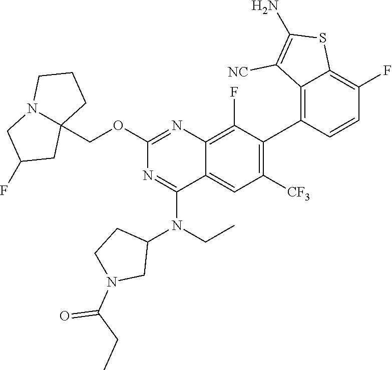 [SMILES: CCC(=O)N1CCC(N(CC)c2nc(OCC34CCCN3CC(F)C4)nc3c(F)c(-c4ccc(F)c5sc(N)c(C#N)c45)c(C(F)(F)F)cc23)C1] | |
| 328 | 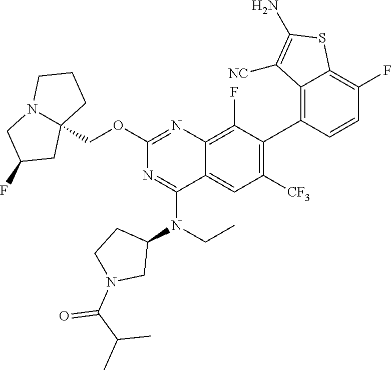 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@@H]1CCN(C(=O)C(C)C)C1] | LCMS m/z [M + 1]: 746.3 1H NMR (400 MHz, CD3OD) δ 8.24 (s, 1H), 7.18- 7.26 (m, 1H), 7.01 (t, 1H), 5.37 (s, 0.5H), 5.24 (s, 0.5H), 4.99-5.11 (m, 1H), 4.22-4.38 (m, 2H), 3.80- 4.19 (m, 4H), 3.62-3.75 (m, 1H), 3.40-3.61 (m, 1H), 3.19-3.29 (m, 3H), 2.98-3.09 (m, 1H), 2.75-2.89 (m, 1H), 2.11-2.56 (m, 5H), 1.85-2.06 (m, 3H), 1.36-1.53 (m, 3H), 1.02-1.21 (m, 6H). |
| 328a | 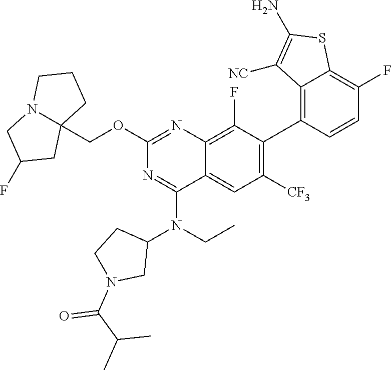 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C(C)C)C1] | |
| 329 | 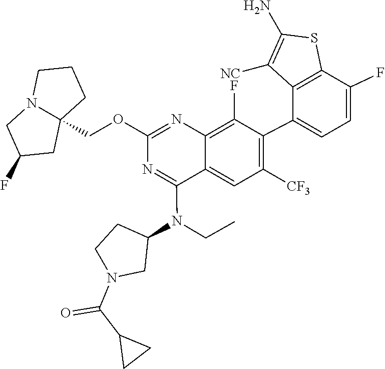 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@@H]1CCN(C(=O)C2CC2)C1] | LCMS m/z [M + 1]: 744.1 1H NMR (400 MHz, CD3OD) δ 825 (s, 1H), 7.18- 7.26 (m, 1H), 7.01 (t, 1H), 5.37 (s, 0.5H), 5.23 (s, 0.5H), 5.00-5.13 (m, 1H), 4.29-4.35 (m, 1H), 4.21- 4.28 (m, 1H), 3.97-4.14 (m, 1H), 3.55-3.96 (m, 4H), 3.33-3.51 (m, 1H), 3.14-3.29 (m, 3H), 2.98-3.08 (m, 1H), 2.08-2.61 (m, 5H), 1.95-2.06 (m, 2H), 1.78-1.93 (m, 2H), 1.41-1.53 (m, 3H), 0.80-0.97 (m, 4H). |
| 329a | 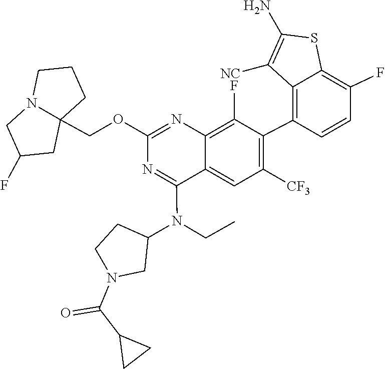 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CC2)C1] | |
| 330 | 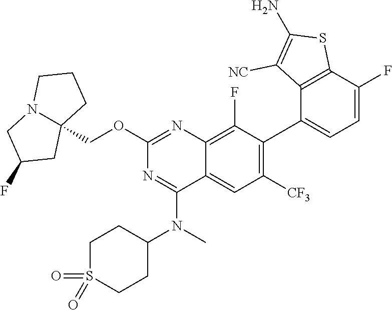 [SMILES: CN(c1nc(OC[C@@]23CCCN2C[C@H](F)C3)nc2c(F)c(-c3ccc(F)c4sc(N)c(C#N)c34)c(C(F)(F)F)cc12)C1CCS(=O)(=O)CC1] | LCMS m/z [M + 1]: 725.3 1H NMR (400 MHz, CD3OD) δ 8.33 (s, 1H), 7.23- 7.20 (m, 1H), 7.04-7.00 (m, 1H), 5.53 (s, 0.50H), 5.33 (s, 0.49H), 4.83-4.76 (m, 1H), 4.60-4.47 (m, 3H), 3.78-3.58 (m, 3H), 3.51-3.45 (m, 4H), 3.28 (s, 1H), 3.21-3.13 (m, 2H), 2.79-2.02 (m, 10H). |
| 330a | 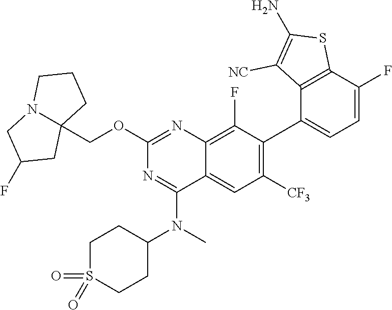 [SMILES: CN(c1nc(OCC23CCCN2CC(F)C3)nc2c(F)c(-c3ccc(F)c4sc(N)c(C#N)c34)c(C(F)(F)F)cc12)C1CCS(=O)(=O)CC1] | |
| 331 | 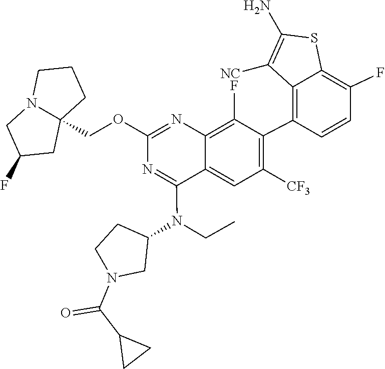 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@H]1CCN(C(=O)C2CC2)C1] | LCMS m/z [M + 1]: 744.3 1H NMR (400 MHz, CD3OD) δ 8.25 (d, 1H), 7.22 (m, 1H), 7.01 (m, 1H), 5.38 (s, 0.5H), 5.24 (s, 0.5H), 5.14-4.99 (m, 1H), 4.27 (m, 2H), 4.14-3.98 (m, 1H), 3.94-3.56 (m, 4H), 3.46 (m, 1H), 3.23 (s, 3H), 3.03 (d, 1H), 2.58-2.09 (m, 5H), 2.00 (m, 2H), 1.87 (m, 2H), 1.52-1.42 (m, 3H), 0.95-0.81 (m, 4H). |
| 331a | 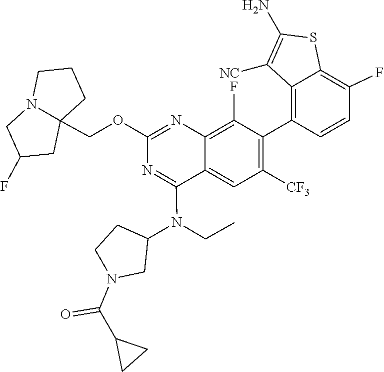 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CC2)C1] | |
| 332 | 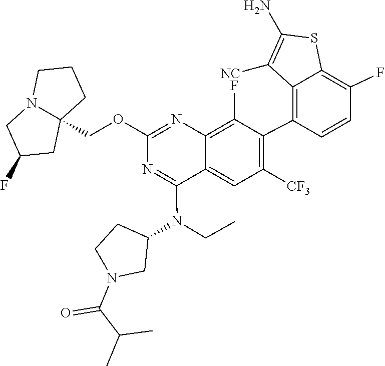 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@H]1CCN(C(=O)C(C)C)C1] | LCMS m/z [M + 1]: 746.3 1H NMR (400 MHz, CD3OD) δ 8.25 (s, 1H), 7.22 (m, 1H), 7.01 (m, 1H), 5.37 (s, 0.5H), 5.24 (s, 0.5H), 5.04 (m, 1H), 4.32-4.11 (m, 2H), 4.03-3.82 (m, 3H), 3.78-3.32 (m, 3H), 3.21 (m, 3H), 3.02 (m, 1H), 2.82 (m, 1H), 2.53-2.09 (m, 5H), 1.95 (m, 3H), 1.49-1.38 (m, 3H), 1.17-1.09 (m, 6H). |
| 332a | 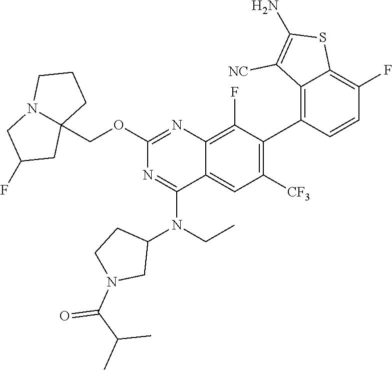 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C(C)C)C1] | |
| 333 | 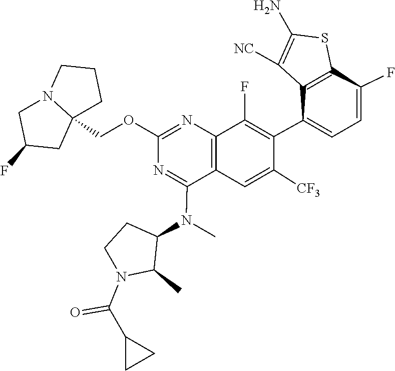 | LCMS m/z [M + 1]: 744.3 1H NMR (400 MHz, CD3OD) δ 8.34 (d, J = 8.4 Hz, 1H), 7.19-7.25 (m, 1H), 6.98-7.05 (m, 1H), 5.29 (d, J = 54.9 Hz, 1H), 4.92-5.12 (m, 2H), 4.19-4.34 (m, 2H), 3.63-3.82 (m, 2H), 3.55-3.63 (m, 3H), 3.45-3.53 (m, 1H), 3.12-3.25 (m, 3H), 2.97-3.05 (m, 1H), 2.34- 2.56 (m, 1H), 2.07-2.28 (m, 3H), 1.82-2.01 (m, 4H), 0.84-1.21 (m, 7H). |
| 333a | 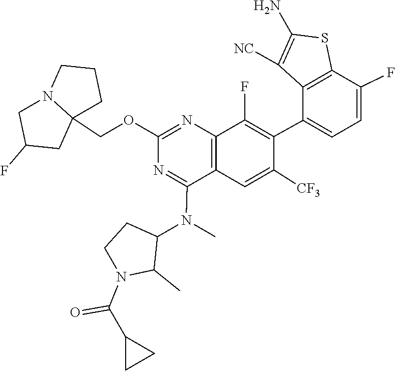 [SMILES: CC1C(N(C)c2nc(OCC34CCCN3CC(F)C4)nc3c(F)c(-c4ccc(F)c5sc(N)c(C#N)c45)c(C(F)(F)F)cc23)CCN1C(=O)C1CC1] | |
| 334 | 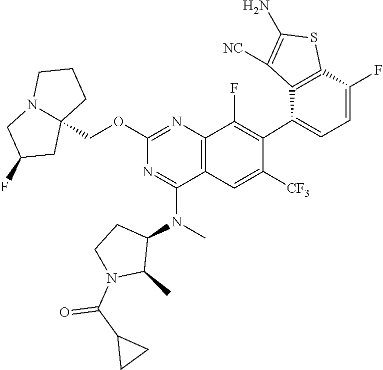 | LCMS m/z [M + 1]: 744.3 1H NMR (400 MHz, CD3OD) δ 8.35 (s, 1H), 7.18- 7.25 (m, 1H), 6.96-7.05 (m, 1H), 5.29 (d, J = 53.2 Hz, 1H), 5.09-5.16 (m, 2H), 4.21-4.32 (m, 2H), 3.56- 3.80 (m, 5H), 3.45-3.52 (m, 1H), 3.11-3.23 (m, 3H), 2.99-3.06 (m, 1H), 2.40-2.51 (m, 1H), 2.12-2.28 (m, 2H), 1.81-2.07 (m, 5H), 0.82-1.25 (m, 7H). |
| 335 | 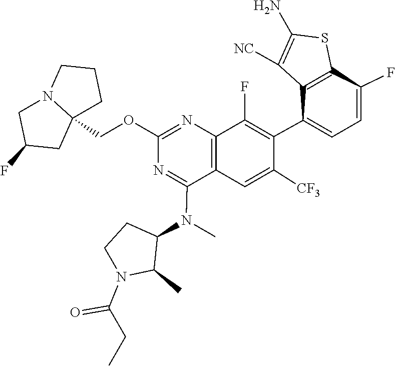 | LCMS m/z [M + 1]: 732.3 1H NMR (400 MHz, CD3OD) δ 8.32 (s, 1H), 7.19- 7.25 (m, 1H), 6.99-7.05 (m, 1H), 5.29 (d, J = 52.9 Hz, 1H), 4.95-5.01 (m, 1H), 4.75-4.82 (m, 1H), 4.21- 4.27 (m, 1H), 3.50-3.81 (m, 6H), 3.12-3.25 (m, 3H), 2.96-3.05 (m, 1H), 2.53-2.66 (m, 1H), 2.34-2.46 (m, 3H), 2.20-2.26 (m, 1H), 2.06-2.13 (m, 1H), 1.94-2.01 (m, 2H), 1.04-1.21 (m, 6H). |
| 335a | 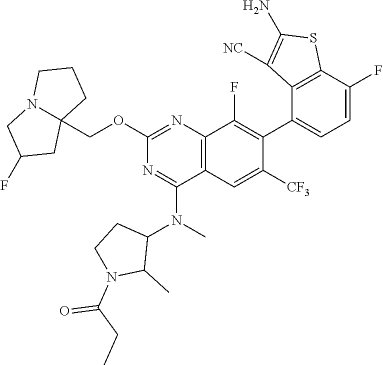 [SMILES: CCC(=O)N1CCC(N(C)c2nc(OCC34CCCN3CC(F)C4)nc3c(F)c(-c4ccc(F)c5sc(N)c(C#N)c45)c(C(F)(F)F)cc23)C1C] | |
| 336 | 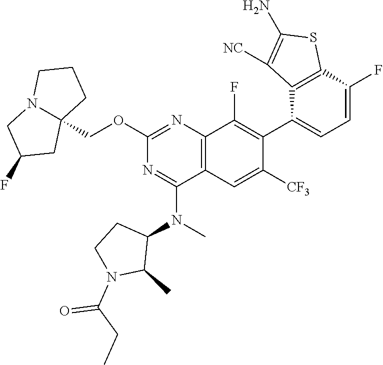 | LCMS m/z [M + 1]: 732.3 1H NMR (400 MHz, CD3OD) δ 8.34 (s, 1H), 7.19- 7.25 (m, 1H), 6.98-7.04 (m, 1H), 5.30 (d, J = 54.1 Hz, 1H), 4.94-5.01 (m, 1H), 4.77-4.84 (m, 1H), 4.20- 4.26 (m, 1H), 3.69-3.82 (m, 1H), 3.48-3.67 (m, 5H), 3.12-3.26 (m, 3H), 2.95-3.05 (m, 1H), 2.58-2.72 (m, 1H), 2.20-2.47 (m, 5H), 1.85-2.10 (m, 4H), 1.06-1.21 (m, 6H). |
| 337 | 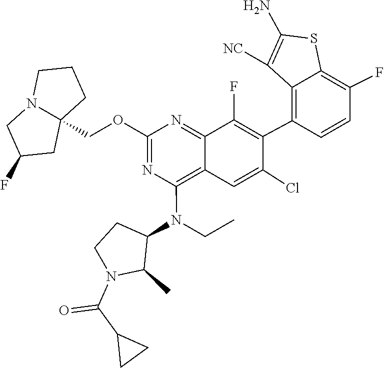 [SMILES: CCN(c1nc(OC[C@@]23CCCN2C[C@H](F)C3)nc2c(F)c(-c3ccc(F)c4sc(N)c(C#N)c34)c(Cl)cc12)[C@@H]1CCN(C(=O)C2CC2)[C@@H]1C] | LCMS m/z [M + 1]: 724.3 1H NMR (400 MHz, CD3OD) δ 8.12 (s, 1H), 7.22- 7.26 (m, 1H), 7.06 (t, J = 8.6 Hz, 1H), 5.56 (d, J = 53.6 Hz, 1H), 4.94-5.05 (m, 1H), 4.59-4.74 (m, 2H), 3.67-4.04 (m, 7H), 3.42-3.52 (m, 1H), 2.31- 2.77(m, 7H), 2.14-2.20 (m, 1H), 1.78-1.85 (m, 1H),1.33-1.38 (m, 3H), 1.13-1.29 (m, 2H), 0.86-0.95 (m, 6H). |
| 337a | 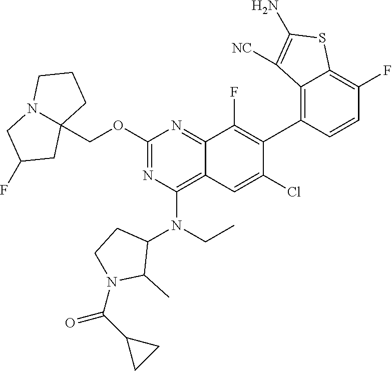 [SMILES: CCN(c1nc(OCC23CCCN2CC(F)C3)nc2c(F)c(-c3ccc(F)c4sc(N)c(C#N)c34)c(Cl)cc12)C1CCN(C(=O)C2CC2)C1C] | |
| 338 | 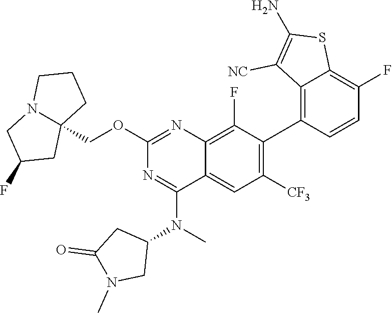 [SMILES: CN1C[C@@H](N(C)c2nc(OC[C@@]34CCCN3C[C@H](F)C4)nc3c(F)c(-c4ccc(F)c5sc(N)c(C#N)c45)c(C(F)(F)F)cc23)CC1=O] | LCMS m/z [M + 1]: 690.1 1H NMR (400 MHz, CD3OD) δ 8.27 (s, 1H), 7.20 (t, 1H), 7.01 (t, 1H), 5.38 (s, 0.5H), 5.39-5.30 (m, 1H), 5.25 (s, 0.5H), 4.30-4.20 (m, 2H), 4.00-3.93 (m, 1H), 3.73-3.63 (m, 1H), 3.46 (s, 3H), 3.39-3.33 (m, 1H), 3.20-3.11 (m, 1H), 3.07-3.00 (m, 1H), 2.95-2.89 (m, 3H), 2.86-2.78 (m, 1H), 2.39-2.13 (m, 4H), 2.05-1.84 (m, 4H). |
| 338a | 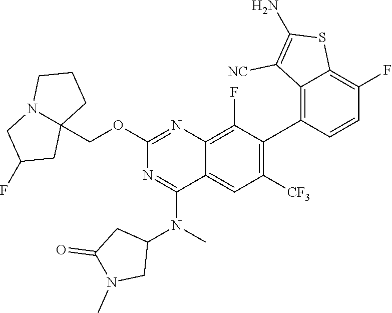 [SMILES: CN1CC(N(C)c2nc(OCC34CCCN3CC(F)C4)nc3c(F)c(-c4ccc(F)c5sc(N)c(C#N)c45)c(C(F)(F)F)cc23)CC1=O] | |
| 339 | 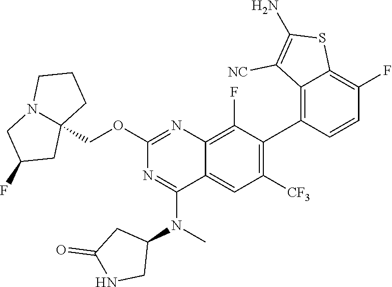 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@H]1CNC(=O)C1] | |
| 339a | 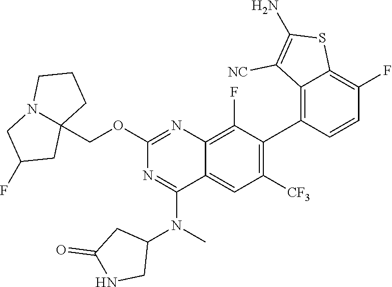 [SMILES: CN(c1nc(OCC23CCCN2CC(F)C3)nc2c(F)c(-c3ccc(F)c4sc(N)c(C#N)c34)c(C(F)(F)F)cc12)C1CNC(=O)C1] | |
| 340 | 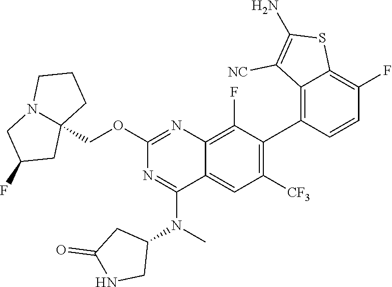 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@@H]1CNC(=O)C1] | LCMS m/z [M + 1]: 676.2 1H NMR (400 MHz, CD3OD) δ 8.30 (s, 1H), 7.20- 7.23 (m, 1H), 7.01 (t, 1H), 5.47 (s, 0.5H), 5.39 (s, 0.5H), 5.33-5.38 (s, 1H), 4.35-4.46 (m, 2H), 3.93 (t, 1H), 3.62-3.67 (m, 1H), 3.47-3.52 (m, 4H), 3.12-3.18 (m, 2H), 2.83-2.90 (m, 1H), 2.72-2.78 (m, 1H), 2.25- 2.49 (m, 3H), 2.12-2.16 (m, 2H), 1.93-2.02 (m, 2H). |
| 341 | 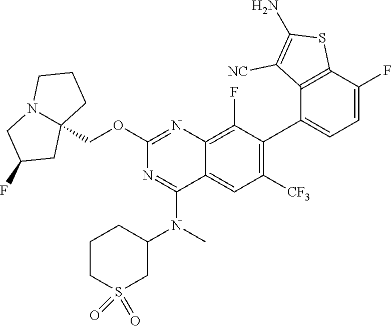 [SMILES: CN(c1nc(OC[C@@]23CCCN2C[C@H](F)C3)nc2c(F)c(-c3ccc(F)c4sc(N)c(C#N)c34)c(C(F)(F)F)cc12)C1CCCS(=O)(=O)C1] | LCMS m/z [M + 1]: 725.0 1H NMR (400 MHz, CD3OD) δ 8.32 (d, 1H), 7.19- 7.23 (m, 1H), 7.00 (t, 1H), 5.39 (s, 0.5H), 5.25 (s, 1.5H), 4.41-4.46 (m, 1H), 4.27-4.35 (m, 1H), 3.51- 3.55 (m, 2H), 3.48 (d, 3H), 3.34-3.39 (m, 1H), 3.04- 3.25 (m, 5H), 2.27-2.46 (m, 2H), 1.98-2.18 (m, 8H). |
| 341a | 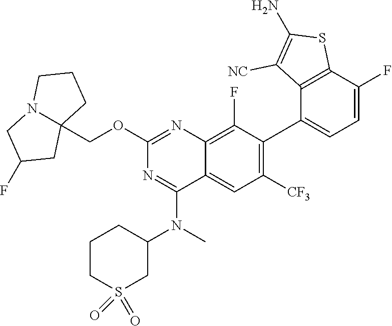 [SMILES: CN(c1nc(OCC23CCCN2CC(F)C3)nc2c(F)c(-c3ccc(F)c4sc(N)c(C#N)c34)c(C(F)(F)F)cc12)C1CCCS(=O)(=O)C1] | |
| 342 | 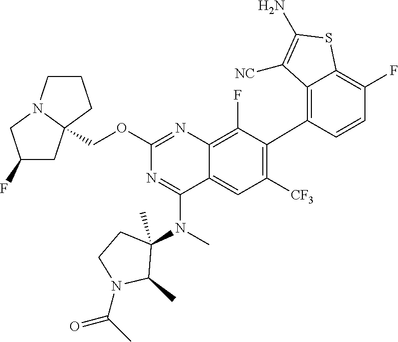 [SMILES: CC(=O)N1CC[C@@](C)(N(C)c2nc(OC[C@@]34CCCN3C[C@H](F)C4)nc3c(F)c(-c4ccc(F)c5sc(N)c(C#N)c45)c(C(F)(F)F)cc23)[C@H]1C] | LCMS m/z [M + 1]: 732.1 1H NMR (400 MHz, CD3OD) δ 8.24 (s, 1H), 7.22 (t, 1H), 7.01 (t, 1H), 5.43 (d, 0.5H), 5.30 (d, 0.5H), 5.14-5.01 (m, 1H), 4.43-4.24 (m, 2H), 3.72 (t, 1H), 3.64-3.48 (m, 5H), 3.24-3.03 (m, 2H), 2.84-2.58 (m, 2H), 2.46-1.93 (m, 10H), 1.66 (s, 3H), 1.10-0.99 (m, 3H). |
| 342a | 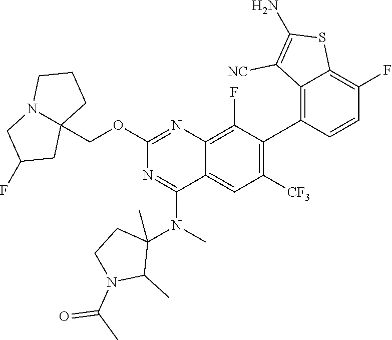 [SMILES: CC(=O)N1CCC(C)(N(C)c2nc(OCC34CCCN3CC(F)C4)nc3c(F)c(-c4ccc(F)c5sc(N)c(C#N)c45)c(C(F)(F)F)cc23)C1C] | |
| 343 | 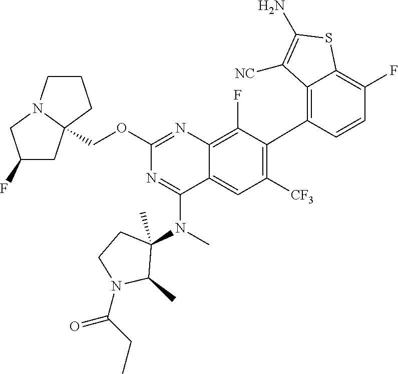 [SMILES: CCC(=O)N1CC[C@@](C)(N(C)c2nc(OC[C@@]34CCCN3C[C@H](F)C4)nc3c(F)c(-c4ccc(F)c5sc(N)c(C#N)c45)c(C(F)(F)F)cc23)[C@H]1C] | LCMS m/z [M + 1]: 746.1 1H NMR (400 MHz, CD3OD) δ 8.24 (s, 1H), 7.22 (t, 1H), 7.00 (t, 1H), 5.42 (d, 0.5H), 5.30 (d, 0.5H), 5.12-4.95 (m, 1H), 4.20-4.23 (m, 2H), 3.70 (t, 1H), 3.61-3.42 (m, 5H), 3.24-3.03 (m, 2H), 2.82-2.53 (m, 2H), 2.46-2.19 (m, 5H), 2.15-1.85 (m, 4H), 1.65 (d, 3H), 1.20-0.98 (m, 6H). |
| 343a | 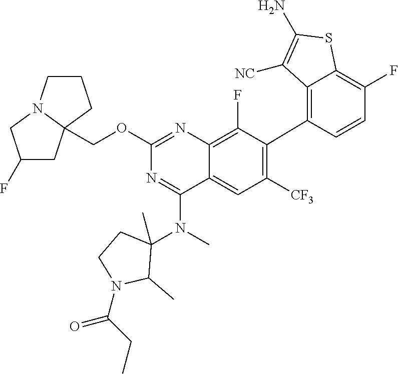 [SMILES: CCC(=O)N1CCC(C)(N(C)c2nc(OCC34CCCN3CC(F)C4)nc3c(F)c(-c4ccc(F)c5sc(N)c(C#N)c45)c(C(F)(F)F)cc23)C1C] | |
| 344 | 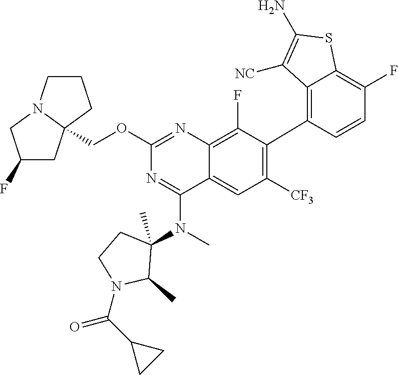 [SMILES: C[C@H]1N(C(=O)C2CC2)CC[C@@]1(C)N(C)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 758.2 1H NMR (400 MHz, CD3OD) δ 8.24 (s, 1H), 7.22 (t, 1H), 7.01 (t, 1H), 5.41 (d, 0.5H), 5.33 (d, 0.5H), 5.16-5.03 (m, 1H), 4.37-4.23 (m, 2H), 3.93-3.74 (m, 1H), 3.57-3.49 (m, 4H), 3.42-3.35 (m, 1H), 3.25-3.02 (m, 3H), 2.78-2.65 (m, 1H), 2.43-2.17 (m, 4H), 2.09- 1.81 (m, 5H), 1.68 (s, 3H), 1.16 (d, 1H), 1.11-0.85 (m, 5H). |
| 344a | 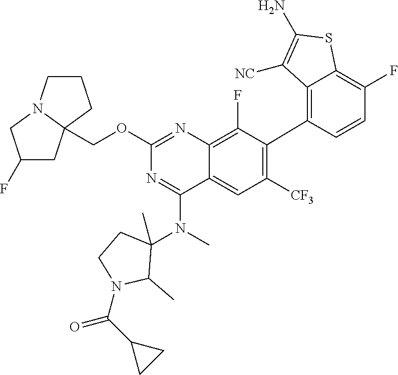 [SMILES: CC1N(C(=O)C2CC2)CCC1(C)N(C)c1nc(OCC23CCCN2CC(F)C3)nc2c(F)c(-c3ccc(F)c4sc(N)c(C#N)c34)c(C(F)(F)F)cc12] | |
| 345 | 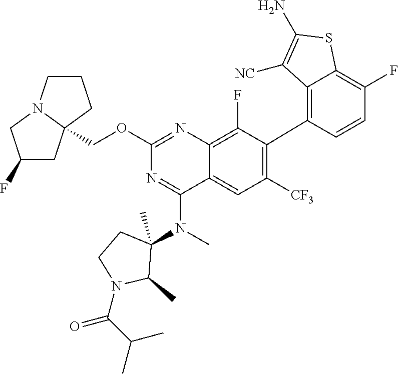 [SMILES: CC(C)C(=O)N1CC[C@@](C)(N(C)c2nc(OC[C@@]34CCCN3C[C@H](F)C4)nc3c(F)c(-c4ccc(F)c5sc(N)c(C#N)c45)c(C(F)(F)F)cc23)[C@H]1C] | LCMS m/z [M + 1]: 760.2 1H NMR (400 MHz, CD3OD) δ 8.48 (s, 1H), 8.26 (s, 1H), 7.22 (t, 1H), 7.01 (t, 1H), 5.55 (d, 0.5H), 5.42 (d, 0.5H), 5.14-5.03 (m, 1H), 4.58-4.36 (m, 3H), 3.85-3.60 (m, 4H), 3.53-3.49 (m, 3H), 2.80-2.75 (m, 2H), 2.65-2.45 (m, 2H), 2.40-2.05 (m, 6H), 1.65 (s, 3H), 1.20 (d, 1H), 1.16-1.05 (m, 6H), 0.99 (d, 2H). |
| 345a | 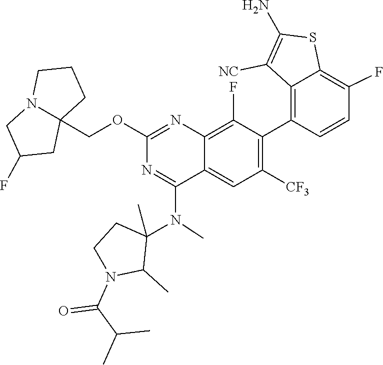 [SMILES: CC(C)C(=O)N1CCC(C)(N(C)c2nc(OCC34CCCN3CC(F)C4)nc3c(F)c(-c4ccc(F)c5sc(N)c(C#N)c45)c(C(F)(F)F)cc23)C1C] | |
| 346 | 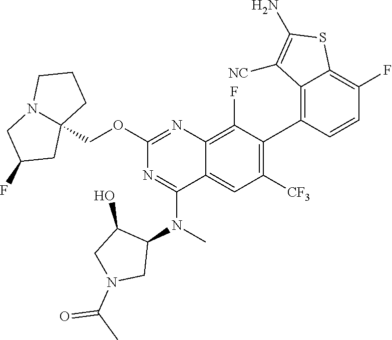 [SMILES: CC(=O)N1C[C@@H](O)[C@@H](N(C)c2nc(OC[C@@]34CCCN3C[C@H](F)C4)nc3c(F)c(-c4ccc(F)c5sc(N)c(C#N)c45)c(C(F)(F)F)cc23)C1] | |
| 346a | 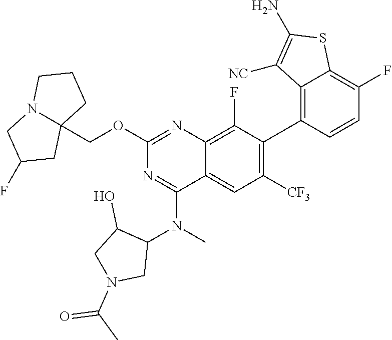 [SMILES: CC(=O)N1CC(O)C(N(C)c2nc(OCC34CCCN3CC(F)C4)nc3c(F)c(-c4ccc(F)c5sc(N)c(C#N)c45)c(C(F)(F)F)cc23)C1] | |
| 347 | 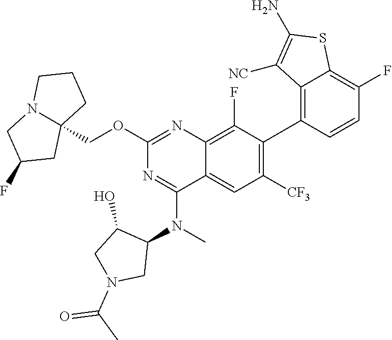 [SMILES: CC(=O)N1C[C@H](O)[C@@H](N(C)c2nc(OC[C@@]34CCCN3C[C@H](F)C4)nc3c(F)c(-c4ccc(F)c5sc(N)c(C#N)c45)c(C(F)(F)F)cc23)C1] | LCMS m/z [M + 1]: 720.21 1H NMR (400 MHz, CD3OD) δ 8.53-8.44 (m, 1H), 7.25-7.11 (m, 1H), 7.01 (t, J = 8.4 Hz, 1H), 5.36 (s, 0.5H), 5.23 (s, 0.5H), 5.13-4.99 (m, 1H), 4.81-4.71 (m, 1H), 4.36-4.21 (m, 2H) 4.15-3.96 (m, 2H), 3.76- 3.61 (m, 1H), 3.56-3.36 (m, 4H), 3.26-3.16 (m, 3H), 3.06-2.97 (m, 1H), 2.40-2.04 (m, 6H), 2.04-1.84 (m, 3H). |
| 348 | 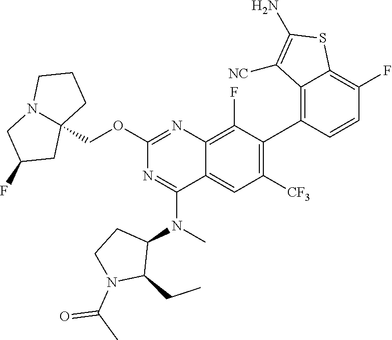 | LCMS m/z [M + 1]: 684.3 1HNMR(400 MHz, CD3OD): δ 8.08(s, 1H), 7.18- 7.24 (m, 1H), 7.01-7.07 (m, 1H), 5.32 (d, J = 54.8 Hz, 1H), 4.92-4.98 (m, 1H ), 4.69-4.78 (m, 1H), 4.57- 4.62 (m, 1H), 4.20-4.38 (m, 2H), 3.45-3.70 (m, 5H), 3.1-3.25 (m, 1H), 2.97-3.08 (m, 1H), 2.35-2.65 (m, 3H), 2.20-2.30 (m, 2H), 1.85-2.15 (m, 6H), 1.0- 1.15 (m, 3H). |
| 348a | 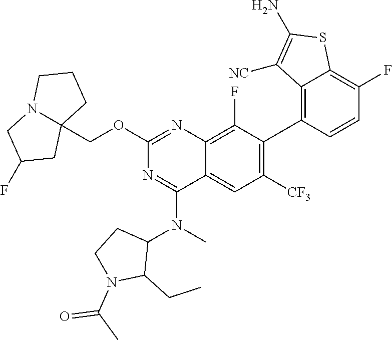 [SMILES: CCC1C(N(C)c2nc(OCC34CCCN3CC(F)C4)nc3c(F)c(-c4ccc(F)c5sc(N)c(C#N)c45)c(C(F)(F)F)cc23)CCN1C(C)=O] | |
| 349 | 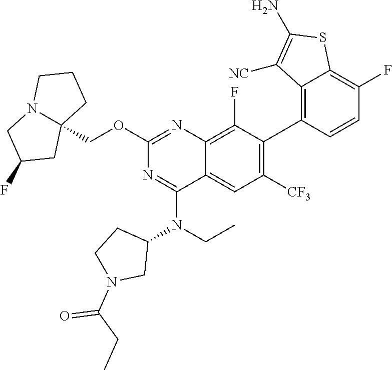 [SMILES: CCC(=O)N1CC[C@H](N(CC)c2nc(OC[C@@]34CCCN3C[C@H](F)C4)nc3c(F)c(-c4ccc(F)c5sc(N)c(C#N)c45)c(C(F)(F)F)cc23)C1] | LCMS m/z [M + 1]: 732.1 1H NMR (400 MHz, CD3OD) δ 8.25 (s, 1H), 7.25- 7.19 (m, 1H), 7.04-6.98 (m, 1H), 5.39 (s, 0.5H), 5.26 (s, 0.5H), 5.07-4.98 (m, 1H), 4.30 (m, 2H), 4.06-3.80 (m, 4H), 3.77-3.54 (m, 2H), 3.25-3.15 (m, 3H), 3.06 (m, 1H), 2.42-2.33 (m, 3H), 2.22 (m, 3H), 1.97 (m, 4H), 1.49-1.42 (m, 3H), 1.17-1.11 (m, 3H). |
| 349a | 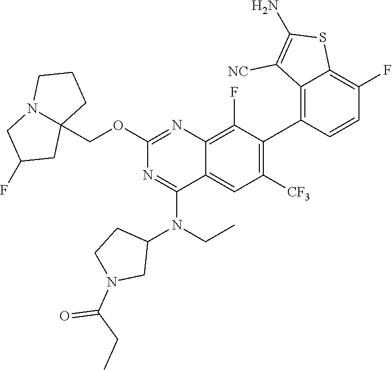 [SMILES: CCC(=O)N1CCC(N(CC)c2nc(OCC34CCCN3CC(F)C4)nc3c(F)c(-c4ccc(F)c5sc(N)c(C#N)c45)c(C(F)(F)F)cc23)C1] | |
| 350 | 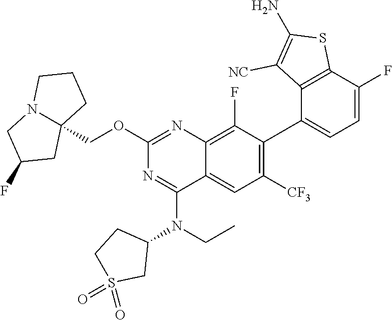 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@H]1CCS(=O)(=O)C1] | LCMS m/z [M + 1]: 725.17 1H NMR (400 MHz, CD3OD) δ 8.25 (s, 1H), 7.24- 7.20 (m, 1H), 7.02 (t, J = 8.8 Hz, 1H), 5.65 (m, 0.5H), 5.52 (m, 0.5H), 5.33 (t, J = 4.8 Hz, 1H), 4.69 (q, J = 12.0 Hz, 2H), 4.10-4.06 (m, 1H), 4.01-3.85 (m, 5H), 3.66-3.43 (m, 4H) , 3.25-3.19 (m, 1H), 2.96- 2.86 (m, 1H), 2.65-2.57 (m, 2H), 2.25-2.43 (m, 1H), 2.36-2.29 (m, 2H), 2.20-2.16 (m, 1H), 1.60 (t, J-6.8 Hz, 3H). |
| 350a | 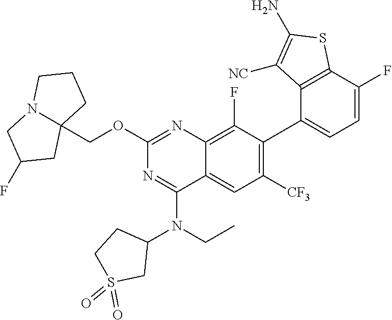 [SMILES: CCN(c1nc(OCC23CCCN2CC(F)C3)nc2c(F)c(-c3ccc(F)c4sc(N)c(C#N)c34)c(C(F)(F)F)cc12)C1CCS(=O)(=O)C1] | |
| 351 | 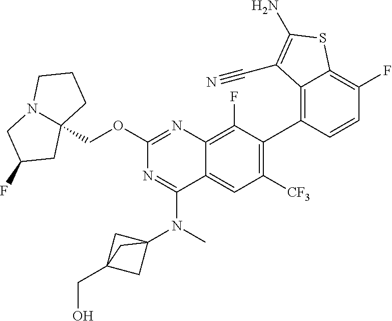 [SMILES: CN(c1nc(OC[C@@]23CCCN2C[C@H](F)C3)nc2c(F)c(-c3ccc(F)c4sc(N)c(C#N)c34)c(C(F)(F)F)cc12)C12CC(CO)(C1)C2] | LCMS m/z [M + 1]: 689.3 1H NMR (400 MHz, CD3OD) δ 8.32 (S, 1H), 7.19- 7.25 (m, 1H), 6.99-7.05 (m, 1H), 5.55 (s, 0.5H), 5.41 (s, 0.5H), 4.87 (s, 3H), 4.60 (s, 1H), 4.48-4.56 (m, 1H), 3.71 (s, 4H), 3.45 (s, 2H), 2.47 (s, 2H), 2.33 (s, 1H), 2.24 (s, 4H), 2.17-2.23 (m, 2H), 1.98-2.06 (m, 2H), 1.56-1.62 (m, 1H). |
| 351a | 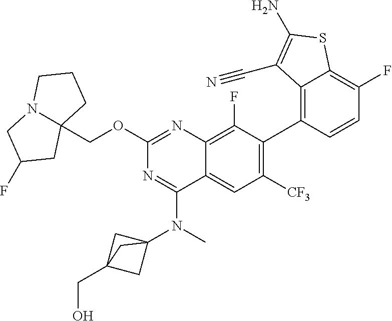 [SMILES: CN(c1nc(OCC23CCCN2CC(F)C3)nc2c(F)c(-c3ccc(F)c4sc(N)c(C#N)c34)c(C(F)(F)F)cc12)C12CC(CO)(C1)C2] | |
| 352 | 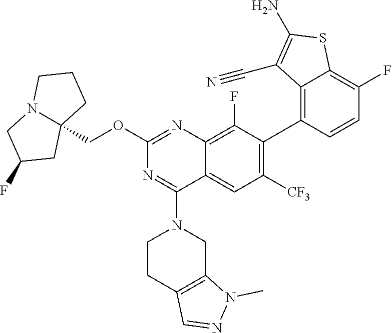 [SMILES: Cn1ncc2c1CN(c1nc(OC[C@@]34CCCN3C[C@H](F)C4)nc3c(F)c(-c4ccc(F)c5sc(N)c(C#N)c45)c(C(F)(F)F)cc13)CC2] | LCMS m/z [M + 1]: 699.1 1H NMR (400 MHz, CD3OD) δ 8.21 (s, 1H), 7.35 (s, 1H), 7.21-7.25 (m, 1H), 7.02 (t, 1H), 5.41 (s, 0.5H), 5.28 (s, 0.5H), 5.01 (s, 2H), 4.40-4.45 (m, 1H), 4.32-4.36 (m, 1H), 4.16-4.21 (m, 1H), 4.00-4.07 (m, 1H), 3.81 (s, 3H), 3.36 (s, 1H), 2.92-3.12 (m, 4H), 2.18-2.43 (m, 4H), 1.82-2.13 (m, 4H). |
| 352a | 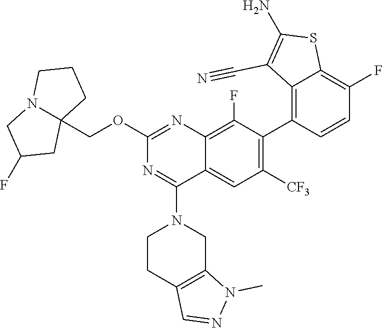 [SMILES: Cn1ncc2c1CN(c1nc(OCC34CCCN3CC(F)C4)nc3c(F)c(-c4ccc(F)c5sc(N)c(C#N)c45)c(C(F)(F)F)cc13)CC2] | |
| 353 | 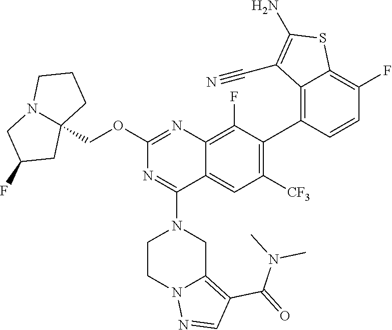 [SMILES: CN(C)C(=O)c1cnn2c1CN(c1nc(OC[C@@]34CCCN3C[C@H](F)C4)nc3c(F)c(-c4ccc(F)c5sc(N)c(C#N)c45)c(C(F)(F)F)cc13)CC2] | LCMS m/z [M + 1]: 756.1 1H NMR (400 MHz, CD3OD) δ 8.24 (s, 1H), 7.81 (s, 1H), 7.18-7.27 (m, 1H), 7.02 (t, 1H), 5.37 (s, 0.5H), 5.34 (s, 2H), 5.24 (s, 0.5H), 4.50 (d, 2H), 4.43 (t, 2H), 4.25-4.38 (m, 2H), 3.15-3.28 (m, 6H), 2.96- 3.14 (m, 4H), 2.22-2.41 (m, 2H), 2.11-2.23 (m, 2H), 1.97-2.05 (m, 2H). |
| 353a | 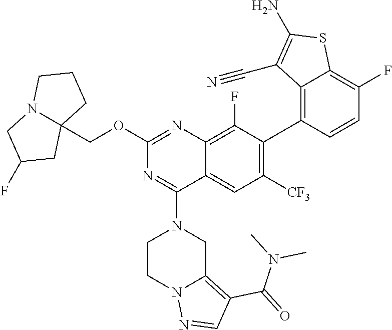 [SMILES: CN(C)C(=O)c1cnn2c1CN(c1nc(OCC34CCCN3CC(F)C4)nc3c(F)c(-c4ccc(F)c5sc(N)c(C#N)c45)c(C(F)(F)F)cc13)CC2] | |
| 354 | 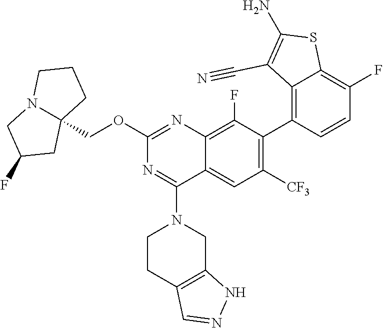 [SMILES: N#Cc1c(N)sc2c(F)ccc(-c3c(C(F)(F)F)cc4c(N5CCc6cn[nH]c6C5)nc(OC[C@@]56CCCN5C[C@H](F)C6)nc4c3F)c12] | LCMS m/z [M + 1]: 685.1 1H NMR (400 MHz, CD3OD) δ 8.26 (s, 1H), 7.52 (s, 1H), 7.26-7.22 (m, 1H), 7.05-7.00 (m, 1H), 5.48 (s, 0.5H), 5.35 (s, 0.5H), 5.09 (s, 1H), 4.60 (s, 1H), 4.54-4.42 (m, 2H), 4.23-4.14 (m, 2H), 2.54-2.26 (m, 3H), 2.16-2.02 (m, 3H). |
| 354a | 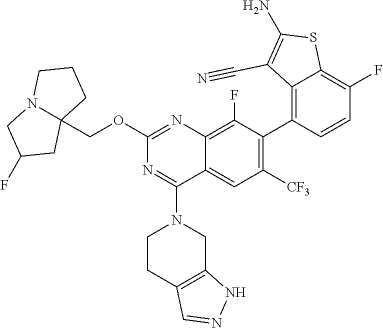 [SMILES: N#Cc1c(N)sc2c(F)ccc(-c3c(C(F)(F)F)cc4c(N5CCc6cn[nH]c6C5)nc(OCC56CCCN5CC(F)C6)nc4c3F)c12] | |
| 355 | 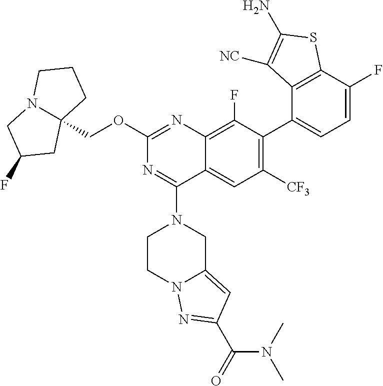 [SMILES: CN(C)C(=O)c1cc2n(n1)CCN(c1nc(OC[C@@]34CCCN3C[C@H](F)C4)nc3c(F)c(-c4ccc(F)c5sc(N)c(C#N)c45)c(C(F)(F)F)cc13)C2] | LCMS m/z [M + 1]: 756.2 HNMR (400 MHz, CD3OD) δ 8.28 (s, 1H), 7.21- 7.25 (m, 1H), 7.03 (t, J = 9.34 Hz, 1H), 6.54 (s, 1H), 5.42-5.55 (d, J = 52.74 Hz, 1H), 5.29 (s, 2H), 4.48- 4.67 (m, 6H), 3.71-3.86 (m, 3H), 3.31-3.38 (m, 4H), 3.09 (s, 3H), 2.07-2.66 (m, 6H). |
| 355a | 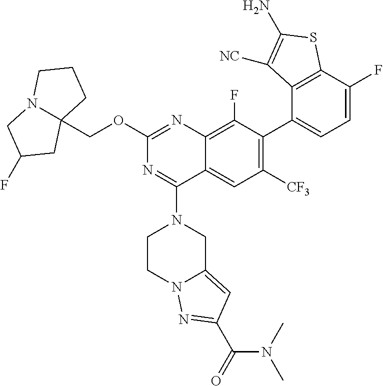 [SMILES: CN(C)C(=O)c1cc2n(n1)CCN(c1nc(OCC34CCCN3CC(F)C4)nc3c(F)c(-c4ccc(F)c5sc(N)c(C#N)c45)c(C(F)(F)F)cc13)C2] | |
| 356 | 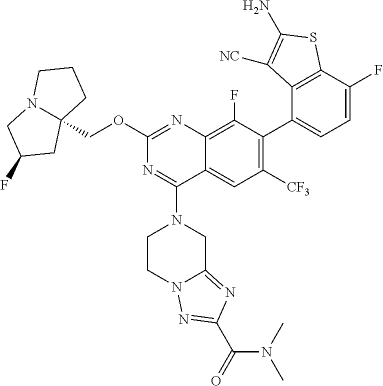 [SMILES: CN(C)C(=O)c1nc2n(n1)CCN(c1nc(OC[C@@]34CCCN3C[C@H](F)C4)nc3c(F)c(-c4ccc(F)c5sc(N)c(C#N)c45)c(C(F)(F)F)cc13)C2] | LCMS m/z [M + 1]: 757.2 1H NMR (400 MHz, CD3OD) δ 8.32 (s, 1H), 7.22- 7.26 (m, 1H), 7.03 (t, J = 9.28 Hz, 1H), 5.57 (d, J = 51.68 Hz, 1H), 5.34 (s, 2H), 4.55-4.78 (m, 5H), 3.85- 4.07 (m, 3H), 3.34-3.51 (m, 2H), 3.26 (s, 3H), 3.14 (s, 3H), 2.13-2.77 (m, 6H). |
| 356a | 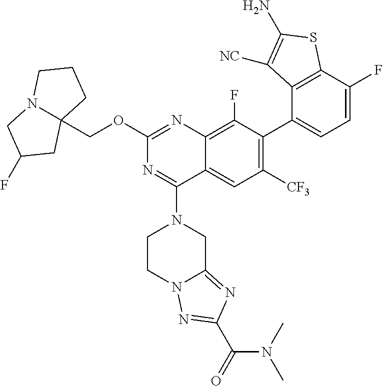 [SMILES: CN(C)C(=O)c1nc2n(n1)CCN(c1nc(OCC34CCCN3CC(F)C4)nc3c(F)c(-c4ccc(F)c5sc(N)c(C#N)c45)c(C(F)(F)F)cc13)C2] | |
| 357 | 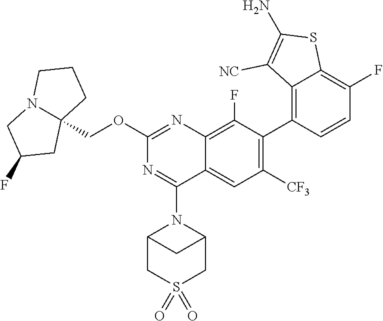 [SMILES: N#Cc1c(N)sc2c(F)ccc(-c3c(C(F)(F)F)cc4c(N5C6CC5CS(=O)(=O)C6)nc(OC[C@@]56CCCN5C[C@H](F)C6)nc4c3F)c12] | LCMS m/z [M + 1]: 709.14 1H NMR (400 MHz, CD3OD) δ 8.01 (s, 1H), 7.25- 7.20 (m, 1H), 7.01 (t, J = 8.8 Hz, 1H), 5.39-5.28 (m, 2.5H), 5.24 (s, 0.5H), 4.60 (s, 1H), 4.35-4.11 (m, 4H), 3.81 (t, J = 17.2 Hz, 2H) 3.21(s, 1H), 3.06-2.99 (m, 1H), 2.47 (d, J = 10.8 Hz, 1H), 2.39-1.80 (m, 8H). |
| 357a | 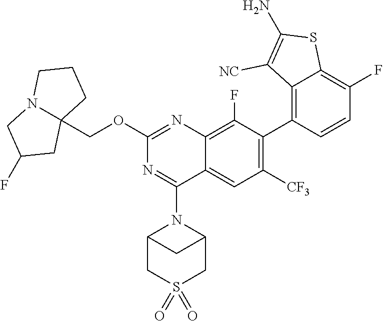 [SMILES: N#Cc1c(N)sc2c(F)ccc(-c3c(C(F)(F)F)cc4c(N5C6CC5CS(=O)(=O)C6)nc(OCC56CCCN5CC(F)C6)nc4c3F)c12] | |
| 358 | 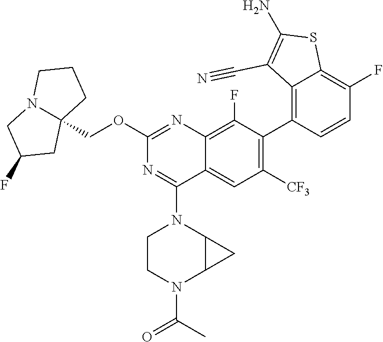 [SMILES: CC(=O)N1CCN(c2nc(OC[C@@]34CCCN3C[C@H](F)C4)nc3c(F)c(-c4ccc(F)c5sc(N)c(C#N)c45)c(C(F)(F)F)cc23)C2CC21] | LCMS m/z [M + 1]: 702.2 1H NMR (400 MHz, CD3OD) δ 8.94 (m, 1H), 7.25- 7.20 (m, 1H), 7.04-7.00 (m, 1H), 5.37 (m, 0.5H), 5.23 (m, 0.5H), 4.32-4.20 (m, 2H), 3.97-3.87 (m, 1H), 3.78-3.35 (m, 5H), 3.24-3.13 (m, 3H), 3.02-2.98 (m, 1H), 2.37-2.12 (m, 6H), 2.04-1.89 (m, 3H), 0.90-0.70 (m, 2H). |
| 358a | 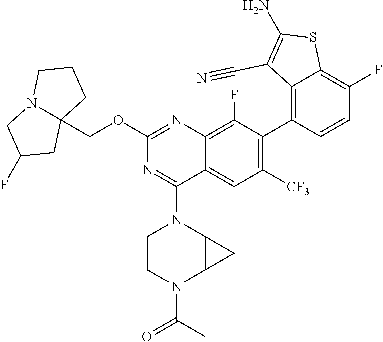 [SMILES: CC(=O)N1CCN(c2nc(OCC34CCCN3CC(F)C4)nc3c(F)c(-c4ccc(F)c5sc(N)c(C#N)c45)c(C(F)(F)F)cc23)C2CC21] | |
| 359 | 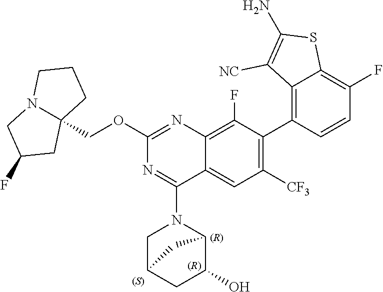 | LCMS m/z [M + 1]: 676.1 1H NMR (400 MHz, CD3OD) δ 8.54 (m, 1H), 7.25- 7.19 (m, 1H), 7.04-6.99 (m, 1H), 5.54 (m, 1H), 5.38 (m, 0.5H), 5.24 (m, 0.5H), 4.63-4.57 (m, 1H), 4.51- 4.48 (m, 1H), 4.33-4.28 (m, 1H), 4.25-4.22 (m, 1H), 4.06 (m, 1H), 3.67-3.64 (m, 1H), 3.49-3.41 (m, 1H), 3.27-3.23 (m, 2H), 3.07-3.00 (m, 1H), 2.76-2.65 (m, 2H), 2.40-1.88 (m, 7H). |
| 360 | 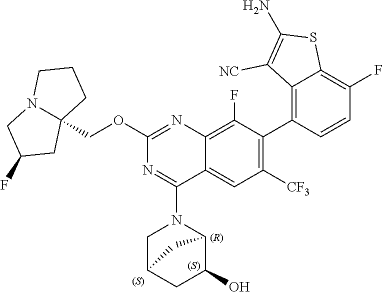 | LCMS m/z [M + 1]: 675.3 1H NMR (400 MHz, CD3OD) δ 8.34 (s, 1H), 7.21- 7.18 (m, 1H), 7.03-6.98 (m, 1H), 5.40 (s, 0.61H), 5.27 (s, 0.59H), 4.60-4.16 (m, 4H), 3.71-3.32 (m, 4H), 3.12-3.07 (m, 1H), 2.83 (s, 1H), 2.31-2.21 (m, 4H), 2.03-1.81 (m, 6H), 1.60-1.53 (m, 1H). |
| 361 | 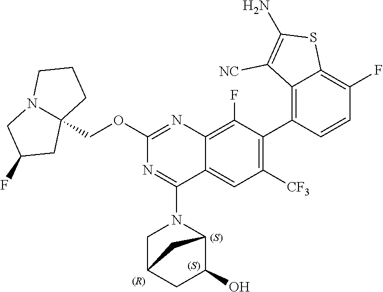 | LCMS m/z [M + 1]: 675.19 1H NMR (400 MHz, CD3OD) δ 8.37-8.29 (m, 1H), 7.22-7.15 (m, 1H), 7.03-6.96 (m, 1H), 5.39 (s, 0.5H), 5.26 (s, 0.5H), 4.60 (s, 1H), 4.43-4.23 (m, 2H), 4.22- 4.00 (m, 2H), 3.60-3.32 (m, 3H), 3.11-3.01 (m, 1H), 2.82 (s, 1H), 2.41-2.16 (m, 3H), 2.08-1.77 (m, 6H), 1.64-1.47 (m, 2H). |
| 362 | 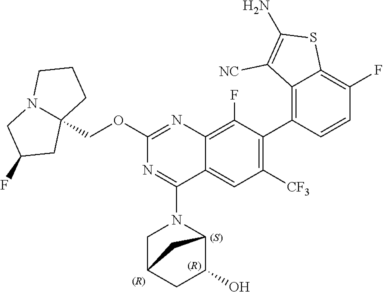 | LCMS m/z [M + 1]: 675.19 1H NMR (400 MHz, CD3OD) δ 8.37-8.29 (m, 1H), 7.22-7.15 (m, 1H), 7.03-6.95 (m, 1H), 5.38 (s, 0.5H), 5.25 (s, 0.5H), 4.89 (s, 1H), 4.37-4.22 (m, 2H), 4.20- 4.06 (m, 2H), 3.61-3.48 (m, 1H), 3.28-3.17 (m, 2H), 3.08-2.99 (m, 1H), 2.82 (m, 1H), 2.44-1.76 (m, 10H), 1.59-1.50 (m, 1H). |
| 362a | 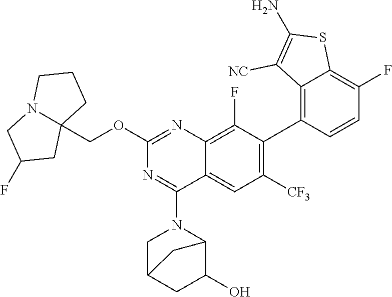 [SMILES: N#Cc1c(N)sc2c(F)ccc(-c3c(C(F)(F)F)cc4c(N5CC6CC(O)C5C6)nc(OCC56CCCN5CC(F)C6)nc4c3F)c12] | |
| 363 | 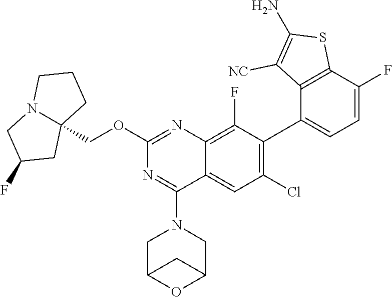 [SMILES: N#Cc1c(N)sc2c(F)ccc(-c3c(Cl)cc4c(N5CC6CC(C5)O6)nc(OC[C@@]56CCCN5C[C@H](F)C6)nc4c3F)c12] | LCMS m/z [M + 1]: 627 1H-NMR (400 MHz, CD3OD-d6): δ 8.41 (s, 1H), 7.20-7.23 (m, 1H), 7.05 (t, J = 9 Hz, 1H), 5.42 (d, J = 25 Hz, 1H), 4.81 (d, J = 6.4 Hz, 2H), 4.47-4.58 (m, 1H), 4.30 (t, 13.6 Hz, 2H), 3.68-3.87 (m, 3H), 3.32- 3.40 (m, 1H), 2.06-2.62 (m, 7H), 1.95 (d, J = 8.4 Hz, 1H). |
| 363a | 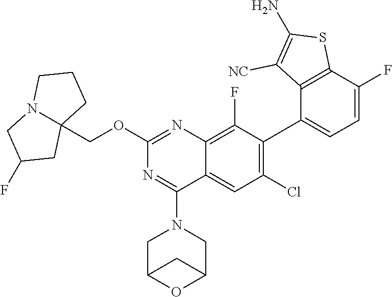 [SMILES: N#Cc1c(N)sc2c(F)ccc(-c3c(Cl)cc4c(N5CC6CC(C5)O6)nc(OCC56CCCN5CC(F)C6)nc4c3F)c12] | |
| 364 | 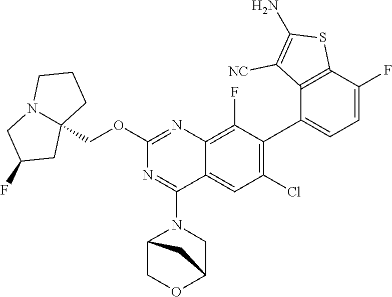 [SMILES: N#Cc1c(N)sc2c(F)ccc(-c3c(Cl)cc4c(N5C[C@H]6C[C@@H]5CO6)nc(OC[C@@]56CCCN5C[C@H](F)C6)nc4c3F)c12] | LCMS m/z [M + 1]: 627.2 1H NMR (400 MHz, CD3OD) δ 8.02-8.03 (d, 1H), 7.17-7.21 (m, 1H), 7.01-7.05 (m, 1H), 5.45 (d, 1H), 5.32-5.36 (m, 0.5H), 5.26 (s, 0.5H), 4.79 (s, 1H), 4.30-4.35 (m, 1H), 4.25 (m, 2H), 4.05-4.10 (m, 1H), 3.91-4.00 (m, 2H, 3.33 (s, 2H), 3.03-3.09 (m, 1H), 2.23-2.36 (m, 2H), 2.00-2.19 (m, 6H), 1.87-1.93 (m, 1H). |
| 364a | 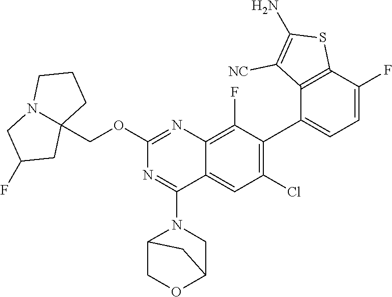 [SMILES: N#Cc1c(N)sc2c(F)ccc(-c3c(Cl)cc4c(N5CC6CC5CO6)nc(OCC56CCCN5CC(F)C6)nc4c3F)c12] | |
| 365 | 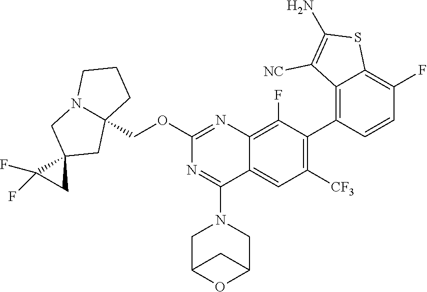 [SMILES: N#Cc1c(N)sc2c(F)ccc(-c3c(C(F)(F)F)cc4c(N5CC6CC(C5)O6)nc(OC[C@@]56CCCN5C[C@]5(CC5(F)F)C6)nc4c3F)c12] | LCMS m/z [M + 1]: 705.0 1H NMR (400 MHz, CD3OD) δ 8.60 (s, 1H), 7.27- 7.17 (m, 1H), 7.01 (t, J = 9.2 Hz, 1H), 4.83-4.76 (m, 2H), 4.59-4.26 (m, 5H), 3.42-3.31 (m, 2H), 3.28-3.16 (m, 2H), 2.96-2.85 (m, 1H), 2.81-2.68 (m, 1H), 2.38- 2.25 (m, 1H), 2.22-2.12 (m, 1H), 2.10-1.72 (m, 5H), 1.54-1.36 (m, 2H). |
| 365a | 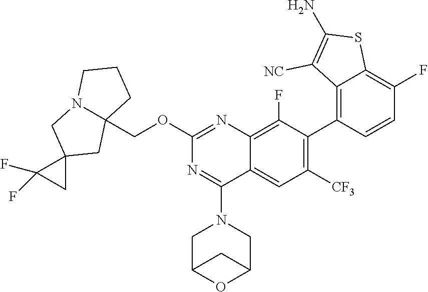 [SMILES: N#Cc1c(N)sc2c(F)ccc(-c3c(C(F)(F)F)cc4c(N5CC6CC(C5)O6)nc(OCC56CCCN5CC5(C6)CC5(F)F)nc4c3F)c12] | |
| 366 | 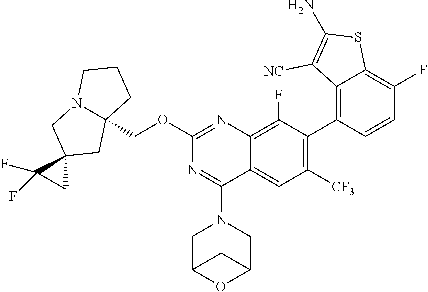 [SMILES: N#Cc1c(N)sc2c(F)ccc(-c3c(C(F)(F)F)cc4c(N5CC6CC(C5)O6)nc(OC[C@@]56CCCN5C[C@@]5(CC5(F)F)C6)nc4c3F)c12] | LCMS m/z [M + 1]: 705.18 1H NMR (400 MHz, CD3OD) δ 8.60 (s, 1H), 7.25- 7.19 (m, 1H), 7.01 (t, J = 8.8 Hz, 1H), 4.82 (d, J = 3.2 Hz, 2H), 4.61-4.47 (m, 2H), 4.44-4.22 (m, 4H), 3.29-3.20 (m, 2H), 3.15-3.07 (m, 1H), 2.93-2.79 (m, 2H), 2.38-2.29 (m, 1H), 2.16-1.88 (m, 6H), 1.50-1.41 (m, 2H). |
| 367 | 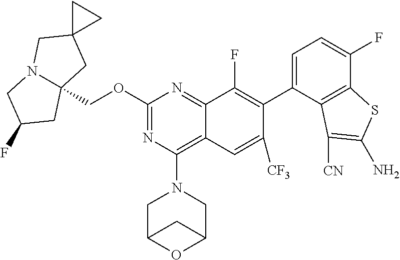 [SMILES: N#Cc1c(N)sc2c(F)ccc(-c3c(C(F)(F)F)cc4c(N5CC6CC(C5)O6)nc(OC[C@]56C[C@@H](F)CN5CC5(CC5)C6)nc4c3F)c12] | |
| 367a | 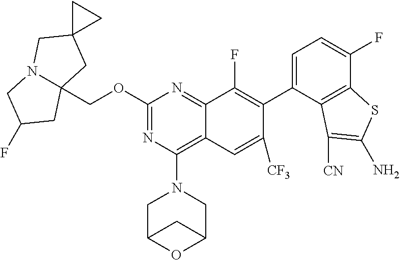 [SMILES: N#Cc1c(N)sc2c(F)ccc(-c3c(C(F)(F)F)cc4c(N5CC6CC(C5)O6)nc(OCC56CC(F)CN5CC5(CC5)C6)nc4c3F)c12] | |
| 368 | 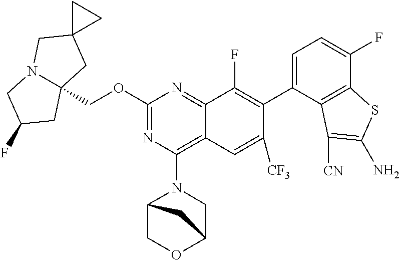 [SMILES: N#Cc1c(N)sc2c(F)ccc(-c3c(C(F)(F)F)cc4c(N5C[C@H]6C[C@@H]5CO6)nc(OC[C@]56C[C@@H](F)CN5CC5(CC5)C6)nc4c3F)c12] | |
| 368a | 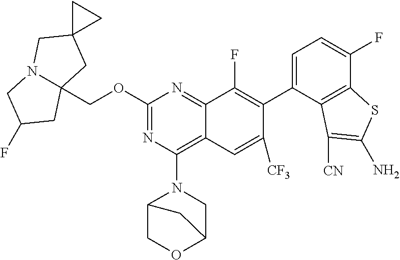 [SMILES: N#Cc1c(N)sc2c(F)ccc(-c3c(C(F)(F)F)cc4c(N5CC6CC5CO6)nc(OCC56CC(F)CN5CC5(CC5)C6)nc4c3F)c12] | |
| 369 | 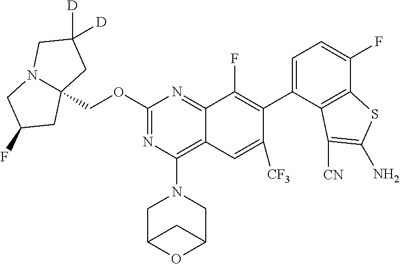 [SMILES: [2H]C1([2H])CN2C[C@H](F)C[C@]2(COc2nc(N3CC4CC(C3)O4)c3cc(C(F)(F)F)c(-c4ccc(F)c5sc(N)c(C#N)c45)c(F)c3n2)C1] | LCMS m/z [M + 1]: 663.2 1H NMR (400 MHz, CD3OD-d4): δ 8.64 (s, 1H), 7.21-7.24 (m, 1H), 7.02 (t, J = 8.62 Hz, 1H), 5.48 (d, J = 54.12 Hz, 1H), 4.31-4.83 (m, 9H), 3.57-3.84 (m, 3H), 1.92-2.79 (m, 6H). |
| 369a | 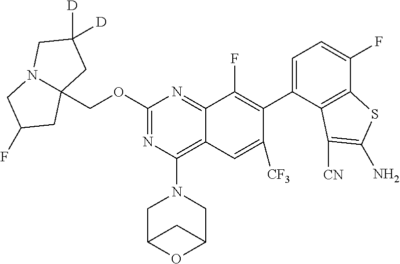 [SMILES: [2H]C1([2H])CN2CC(F)CC2(COc2nc(N3CC4CC(C3)O4)c3cc(C(F)(F)F)c(-c4ccc(F)c5sc(N)c(C#N)c45)c(F)c3n2)C1] | |
| 370 | 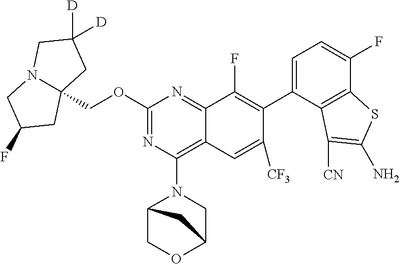 [SMILES: [2H]C1([2H])CN2C[C@H](F)C[C@]2(COc2nc(N3C[C@H]4C[C@@H]3CO4)c3cc(C(F)(F)F)c(-c4ccc(F)c5sc(N)c(C#N)c45)c(F)c3n2)C1] | |
| 370a | 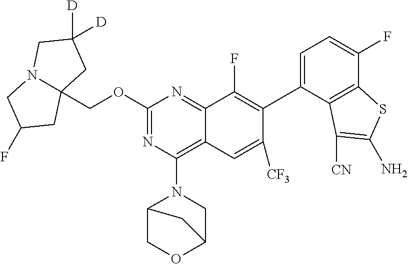 [SMILES: [2H]C1([2H])CN2CC(F)CC2(COc2nc(N3CC4CC3CO4)c3cc(C(F)(F)F)c(-c4ccc(F)c5sc(N)c(C#N)c45)c(F)c3n2)C1] | |
| 371 | 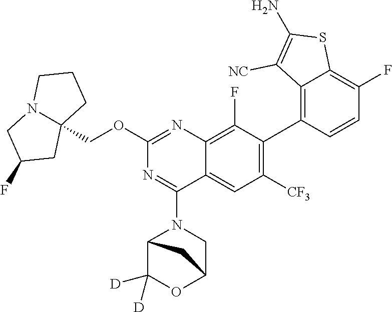 [SMILES: [2H]C1([2H])O[C@@H]2C[C@H]1N(c1nc(OC[C@@]34CCCN3C[C@H](F)C4)nc3c(F)c(-c4ccc(F)c5sc(N)c(C#N)c45)c(C(F)(F)F)cc13)C2] | LCMS m/z [M + 1]: 663.0 1H NMR (400 MHz, CD3OD) 88.29 (s, 1H), 7.20 (s, 1H), 7.00 (t, J = 8.8 Hz, 1H), 5.56-5.44 (m, 1H), 5.40-5.22 (m, 1H), 4.82-4.76 (m, 1H), 4.46-4.06 (m, 3H), 4.04-3.93 (m, 1H), 3.43-3.32 (m, 1H), 3.29-3.20 (m, 2H), 3.10-2.98 (m, 1H), 2.50-1.74 (m, 8H). |
| 371a | 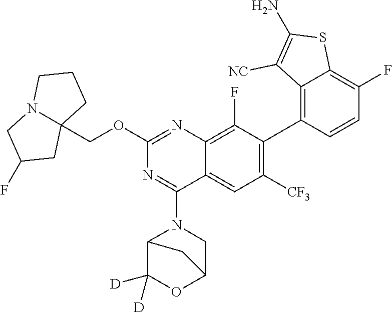 [SMILES: [2H]C1([2H])OC2CC1N(c1nc(OCC34CCCN3CC(F)C4)nc3c(F)c(-c4ccc(F)c5sc(N)c(C#N)c45)c(C(F)(F)F)cc13)C2] | |
| 372 | 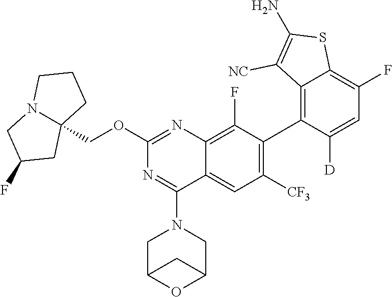 [SMILES: [2H]c1cc(F)c2sc(N)c(C#N)c2c1-c1c(C(F)(F)F)cc2c(N3CC4CC(C3)O4)nc(OC[C@@]34CCCN3C[C@H](F)C4)nc2c1F] | |
| 372a | 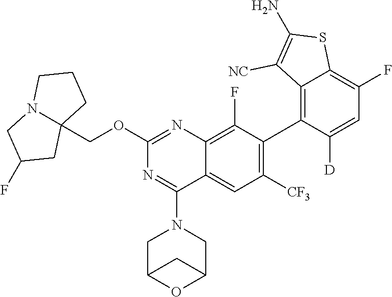 [SMILES: [2H]c1cc(F)c2sc(N)c(C#N)c2c1-c1c(C(F)(F)F)cc2c(N3CC4CC(C3)O4)nc(OCC34CCCN3CC(F)C4)nc2c1F] | |
| 373 | 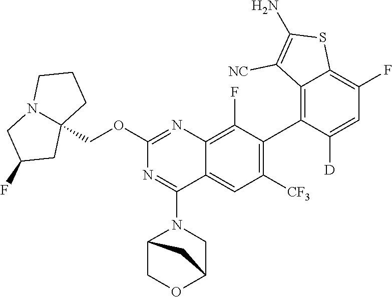 [SMILES: [2H]c1cc(F)c2sc(N)c(C#N)c2c1-c1c(C(F)(F)F)cc2c(N3C[C@H]4C[C@@H]3CO4)nc(OC[C@@]34CCCN3C[C@H](F)C4)nc2c1F] | |
| 373a | 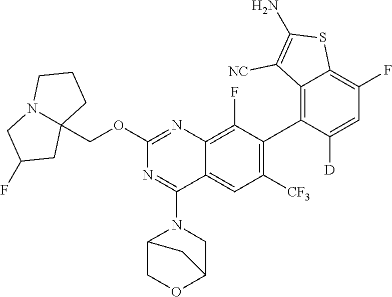 [SMILES: [2H]c1cc(F)c2sc(N)c(C#N)c2c1-c1c(C(F)(F)F)cc2c(N3CC4CC3CO4)nc(OCC34CCCN3CC(F)C4)nc2c1F] | |
| 374 | 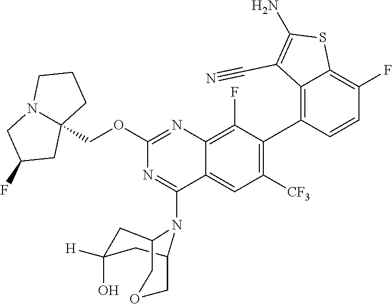 [SMILES: N#Cc1c(N)sc2c(F)ccc(-c3c(C(F)(F)F)cc4c(N5C6COCC5CC(O)C6)nc(OC[C@@]56CCCN5C[C@H](F)C6)nc4c3F)c12] | LCMS m/z [M + 1]: 705.20. 1H NMR (400 MHz, CD3OD) δ 8.56 (s, 1H), 7.25- 7.17 (m, 1H), 7.01 (t, J = 8.9 Hz, 1H), 5.41 (s, 0.5H), 5.28 (s, 0.5H), 4.60 (s, 2H), 4.41-4.26 (m, 2H), 4.13- 3.93 (m, 5H), 3.51-3.32 (m, 3H), 3.15-3.05 (m, 1H), 2.66-2.52 (m, 2H), 2.45-2.14 (m, 3H), 2.11-1.87 (m, 5H). |
| 374a | 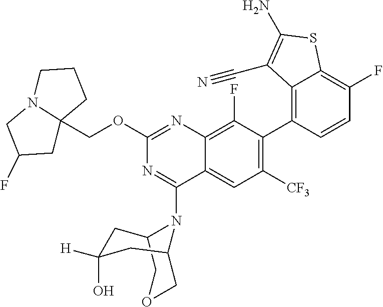 [SMILES: N#Cc1c(N)sc2c(F)ccc(-c3c(C(F)(F)F)cc4c(N5C6COCC5CC(O)C6)nc(OCC56CCCN5CC(F)C6)nc4c3F)c12] | |
| 375 | 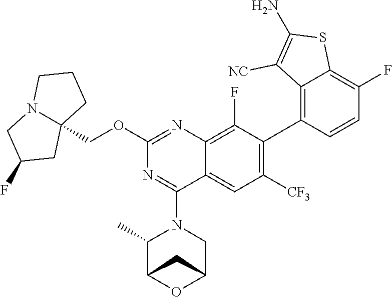 [SMILES: C[C@H]1[C@@H]2C[C@H](CN1c1nc(OC[C@@]34CCCN3C[C@H](F)C4)nc3c(F)c(-c4ccc(F)c5sc(N)c(C#N)c45)c(C(F)(F)F)cc13)O2] | |
| 376 | 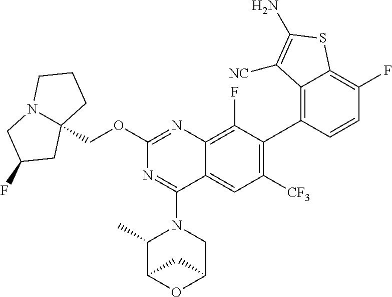 [SMILES: C[C@H]1[C@H]2C[C@@H](CN1c1nc(OC[C@@]34CCCN3C[C@H](F)C4)nc3c(F)c(-c4ccc(F)c5sc(N)c(C#N)c45)c(C(F)(F)F)cc13)O2] | |
| 377 | 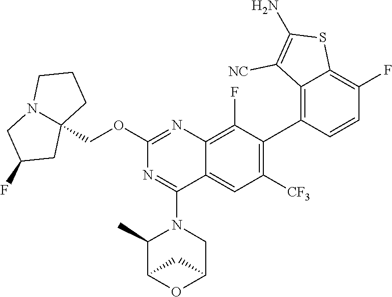 [SMILES: C[C@@H]1[C@H]2C[C@@H](CN1c1nc(OC[C@@]34CCCN3C[C@H](F)C4)nc3c(F)c(-c4ccc(F)c5sc(N)c(C#N)c45)c(C(F)(F)F)cc13)O2] | |
| 378 | 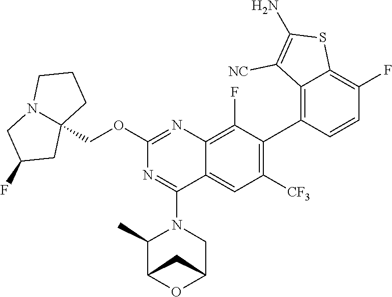 [SMILES: C[C@@H]1[C@@H]2C[C@H](CN1c1nc(OC[C@@]34CCCN3C[C@H](F)C4)nc3c(F)c(-c4ccc(F)c5sc(N)c(C#N)c45)c(C(F)(F)F)cc13)O2] | |
| 378a | 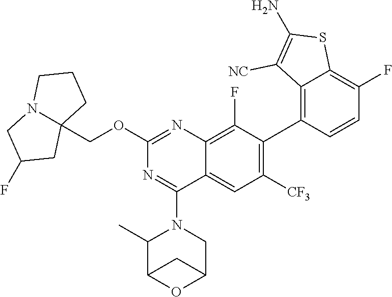 [SMILES: CC1C2CC(CN1c1nc(OCC34CCCN3CC(F)C4)nc3c(F)c(-c4ccc(F)c5sc(N)c(C#N)c45)c(C(F)(F)F)cc13)O2] | |
| 379 | 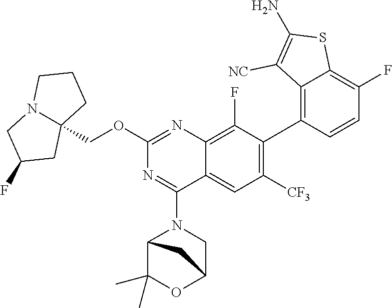 [SMILES: CC1(C)O[C@@H]2C[C@H]1N(c1nc(OC[C@@]34CCCN3C[C@H](F)C4)nc3c(F)c(-c4ccc(F)c5sc(N)c(C#N)c45)c(C(F)(F)F)cc13)C2] | |
| 379a | 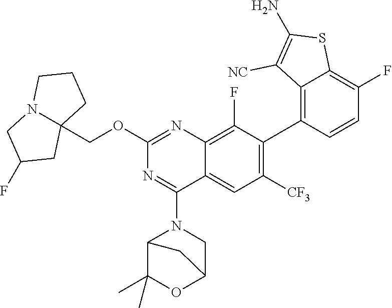 [SMILES: CC1(C)OC2CC1N(c1nc(OCC34CCCN3CC(F)C4)nc3c(F)c(-c4ccc(F)c5sc(N)c(C#N)c45)c(C(F)(F)F)cc13)C2] | |
| 380 | 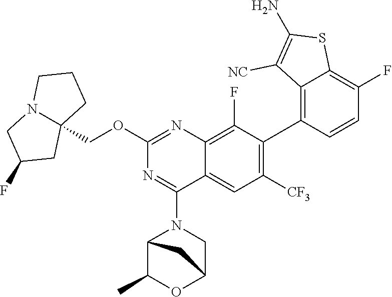 [SMILES: C[C@@H]1O[C@@H]2C[C@H]1N(c1nc(OC[C@@]34CCCN3C[C@H](F)C4)nc3c(F)c(-c4ccc(F)c5sc(N)c(C#N)c45)c(C(F)(F)F)cc13)C2] | |
| 381 | 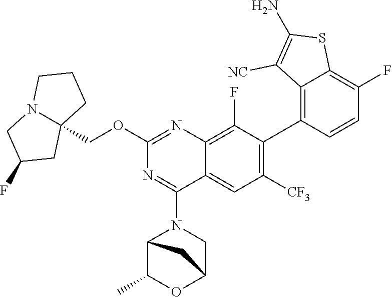 [SMILES: C[C@H]1O[C@@H]2C[C@H]1N(c1nc(OC[C@@]34CCCN3C[C@H](F)C4)nc3c(F)c(-c4ccc(F)c5sc(N)c(C#N)c45)c(C(F)(F)F)cc13)C2] | |
| 381a | 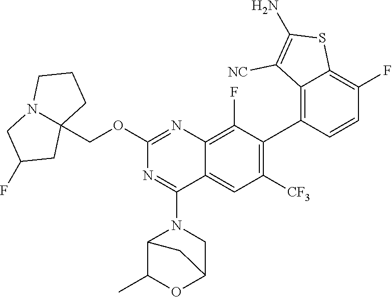 [SMILES: CC1OC2CC1N(c1nc(OCC34CCCN3CC(F)C4)nc3c(F)c(-c4ccc(F)c5sc(N)c(C#N)c45)c(C(F)(F)F)cc13)C2] | |
| 382 | 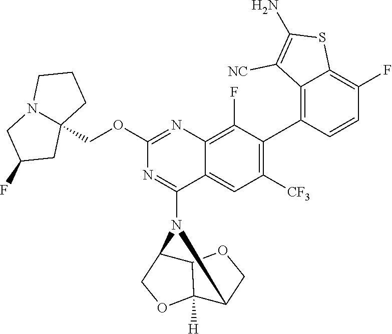 [SMILES: N#Cc1c(N)sc2c(F)ccc(-c3c(C(F)(F)F)cc4c(N5[C@@H]6CO[C@H]7C6OC[C@H]75)nc(OC[C@@]56CCCN5C[C@H](F)C6)nc4c3F)c12] | |
| 382a | 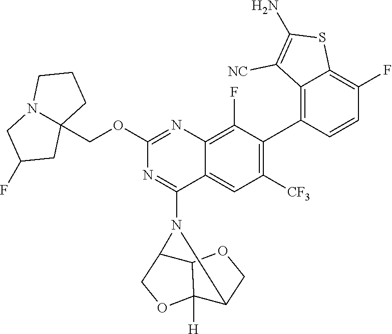 [SMILES: N#Cc1c(N)sc2c(F)ccc(-c3c(C(F)(F)F)cc4c(N5C6COC7C6OCC75)nc(OCC56CCCN5CC(F)C6)nc4c3F)c12] | |
| 383 | 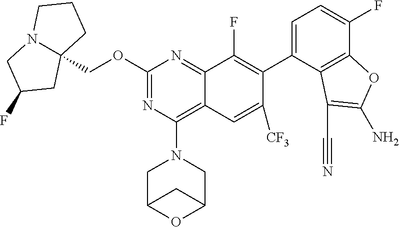 [SMILES: N#Cc1c(N)oc2c(F)ccc(-c3c(C(F)(F)F)cc4c(N5CC6CC(C5)O6)nc(OC[C@@]56CCCN5C[C@H](F)C6)nc4c3F)c12] | |
| 383a | 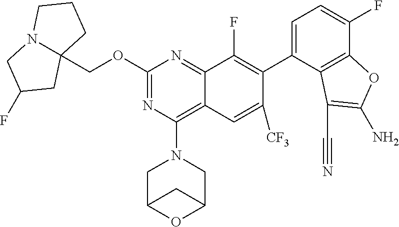 [SMILES: N#Cc1c(N)oc2c(F)ccc(-c3c(C(F)(F)F)cc4c(N5CC6CC(C5)O6)nc(OCC56CCCN5CC(F)C6)nc4c3F)c12] | |
| 384 | 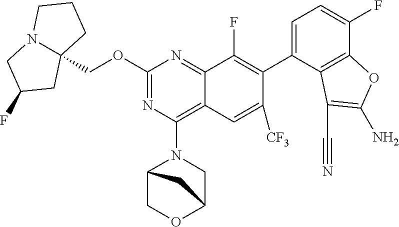 [SMILES: N#Cc1c(N)oc2c(F)ccc(-c3c(C(F)(F)F)cc4c(N5C[C@H]6C[C@@H]5CO6)nc(OC[C@@]56CCCN5C[C@H](F)C6)nc4c3F)c12] | |
| 384a | 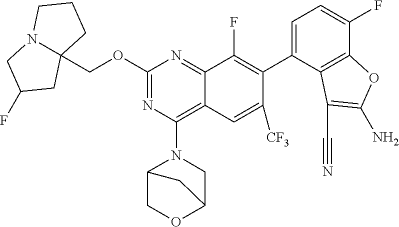 [SMILES: N#Cc1c(N)oc2c(F)ccc(-c3c(C(F)(F)F)cc4c(N5CC6CC5CO6)nc(OCC56CCCN5CC(F)C6)nc4c3F)c12] | |
| 385 | 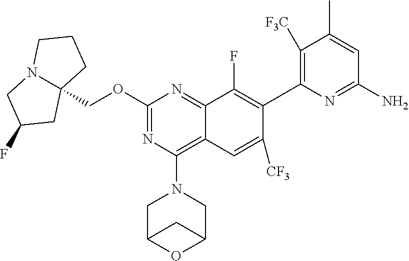 [SMILES: Cc1cc(N)nc(-c2c(C(F)(F)F)cc3c(N4CC5CC(C4)O5)nc(OC[C@@]45CCCN4C[C@H](F)C5)nc3c2F)c1C(F)(F)F] | |
| 385a | 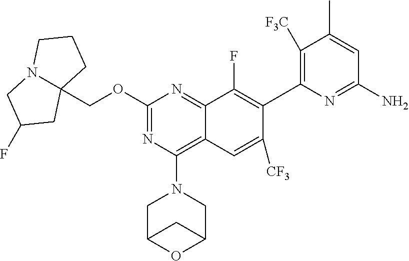 [SMILES: Cc1cc(N)nc(-c2c(C(F)(F)F)cc3c(N4CC5CC(C4)O5)nc(OCC45CCCN4CC(F)C5)nc3c2F)c1C(F)(F)F] | |
| 386 | 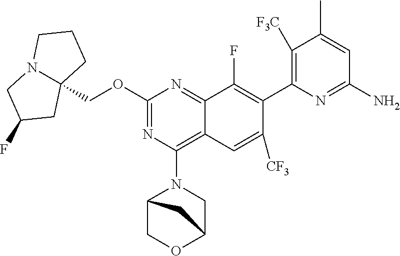 [SMILES: Cc1cc(N)nc(-c2c(C(F)(F)F)cc3c(N4C[C@H]5C[C@@H]4CO5)nc(OC[C@@]45CCCN4C[C@H](F)C5)nc3c2F)c1C(F)(F)F] | |
| 386a | 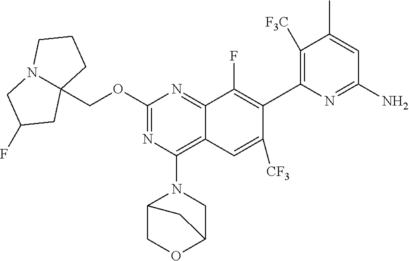 [SMILES: Cc1cc(N)nc(-c2c(C(F)(F)F)cc3c(N4CC5CC4CO5)nc(OCC45CCCN4CC(F)C5)nc3c2F)c1C(F)(F)F] | |
| 387 | 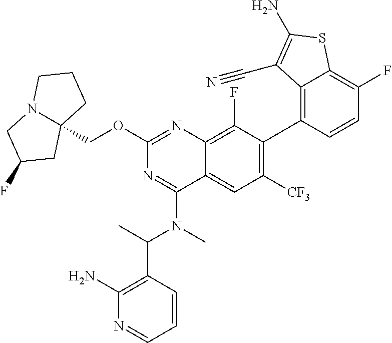 [SMILES: CC(c1cccnc1N)N(C)c1nc(OC[C@@]23CCCN2C[C@H](F)C3)nc2c(F)c(-c3ccc(F)c4sc(N)c(C#N)c34)c(C(F)(F)F)cc12] | |
| 387a | 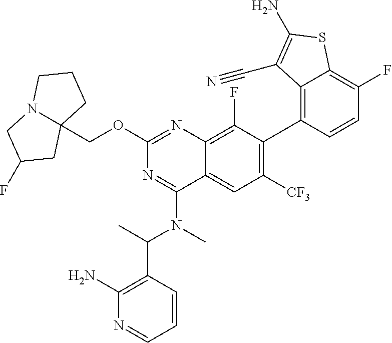 [SMILES: CC(c1cccnc1N)N(C)c1nc(OCC23CCCN2CC(F)C3)nc2c(F)c(-c3ccc(F)c4sc(N)c(C#N)c34)c(C(F)(F)F)cc12] | |
| 388 | 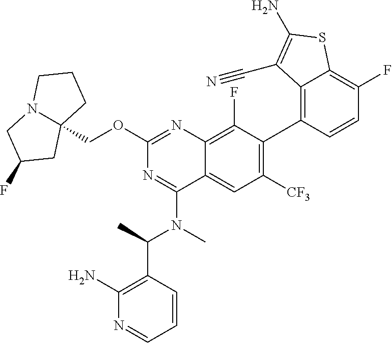 [SMILES: C[C@H](c1cccnc1N)N(C)c1nc(OC[C@@]23CCCN2C[C@H](F)C3)nc2c(F)c(-c3ccc(F)c4sc(N)c(C#N)c34)c(C(F)(F)F)cc12] | |
| 389 | 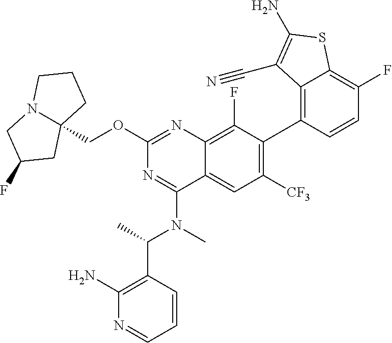 [SMILES: C[C@@H](c1cccnc1N)N(C)c1nc(OC[C@@]23CCCN2C[C@H](F)C3)nc2c(F)c(-c3ccc(F)c4sc(N)c(C#N)c34)c(C(F)(F)F)cc12] | |
| 390 | 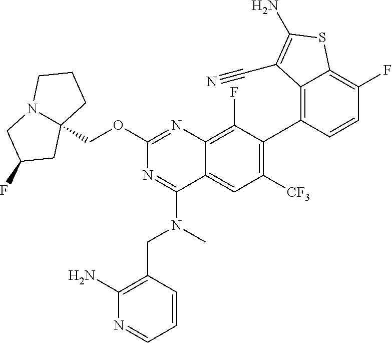 [SMILES: CN(Cc1cccnc1N)c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 699.1 1H NMR (400 MHz, CD3OD) δ 8.40 (s, 1H), 8.24 (s, 1H), 7.95 (d, 1H), 7.56 (d, 1H), 7.19 (t, 1H), 7.00 (t, 1H), 6.73 (t, 1H), 5.54 (s, 0.5H), 5.41 (s, 0.5H), 4.91 (s, 2H), 4.60-4.44 (m, 2H), 3.86-3.68 (m, 3H), 3.58 (s, 3H), 3.38-3.32 (m, 1H), 2.56-2.41 (m, 2H), 2.32-2.19 (m, 3H), 2.08-2.00 (m, 1H). |
| 390a | 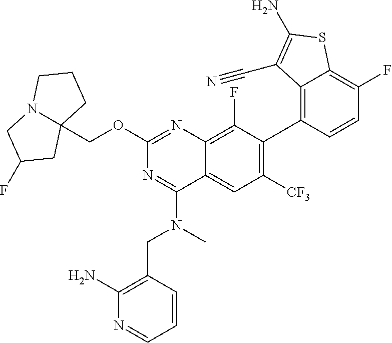 [SMILES: CN(Cc1cccnc1N)c1nc(OCC23CCCN2CC(F)C3)nc2c(F)c(-c3ccc(F)c4sc(N)c(C#N)c34)c(C(F)(F)F)cc12] | |
| 392 | 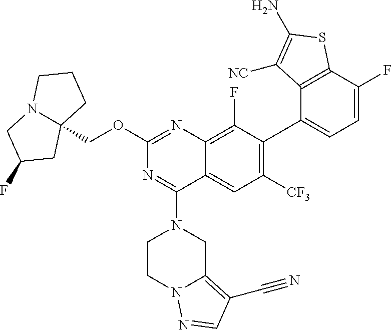 [SMILES: N#Cc1cnn2c1CN(c1nc(OC[C@@]34CCCN3C[C@H](F)C4)nc3c(F)c(-c4ccc(F)c5sc(N)c(C#N)c45)c(C(F)(F)F)cc13)CC2] | LCMS m/z [M + 1]: 710.0 1H NMR (400 MHz, CD3OD) δ 8.29 (s, 1H), 7.95 (s, 1H), 7.24 (m, 1H), 7.07-6.98 (m, 1H), 5.53 (s, 0.5H), 5.49 (s, 1H), 5.40 (s, 0.5H), 5.31 (s, 2H), 4.63- 4.42 (m, 6H), 3.68 (m, 3H), 2.63-2.41 (m, 2H), 2.33 (s, 1H), 2.26-2.16 (m, 2H), 2.05 (m, 1H). |
| 392a | 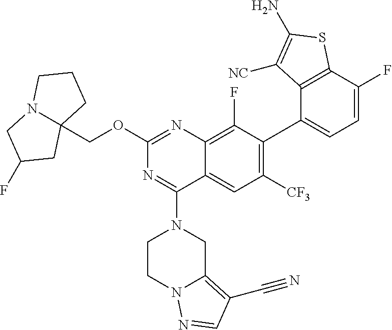 [SMILES: N#Cc1cnn2c1CN(c1nc(OCC34CCCN3CC(F)C4)nc3c(F)c(-c4ccc(F)c5sc(N)c(C#N)c45)c(C(F)(F)F)cc13)CC2] | |
| 393 | 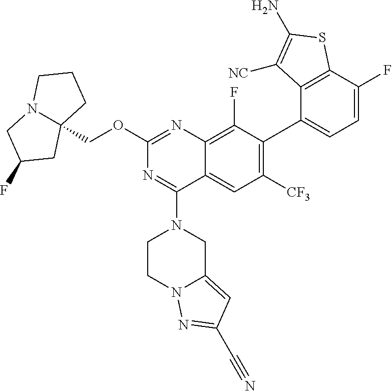 [SMILES: N#Cc1cc2n(n1)CCN(c1nc(OC[C@@]34CCCN3C[C@H](F)C4)nc3c(F)c(-c4ccc(F)c5sc(N)c(C#N)c45)c(C(F)(F)F)cc13)C2] | LCMS m/z [M + 1]: 710.18 1H NMR (400 MHz, DMSO) δ 8.19 (s, 1H), 8.09 (s, 2H), 7.29-7.23 (m, 1H), 7.14 (t, J = 8.6 Hz, 1H), 6.96 (s, 1H), 5.34 (s, 0.5H), 5.234-5.09 (m, 2.5H), 4.63- 4.51 (m, 2H), 4.48-4.39 (m, 1H), 4.28-4.19 (m, 1H), 4.19-4.05 (m, 2H), 3.15-2.99 (m, 3H), 2.87-277 (m, 1H), 2.16-1.99 (m, 3H), 1.89-1.75 (m, 3H). |
| 393a | 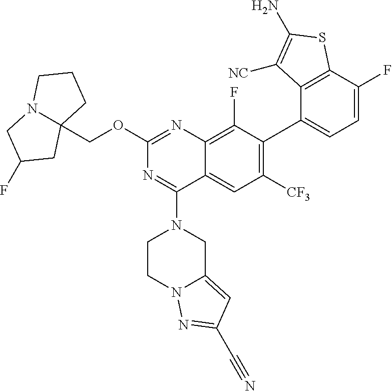 [SMILES: N#Cc1cc2n(n1)CCN(c1nc(OCC34CCCN3CC(F)C4)nc3c(F)c(-c4ccc(F)c5sc(N)c(C#N)c45)c(C(F)(F)F)cc13)C2] | |
| 394 | 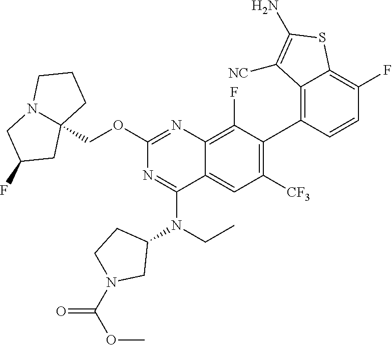 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@H]1CCN(C(=O)OC)C1] | LCMS m/z [M + 1]: 734.1 1H NMR (400 MHz, CD3OD) δ 8.23 (s, 1H), 7.26- 7.16 (m, 1H), 7.01 (t, 1H), 5.42 (s, 0.5H), 5.29 (s, 0.5H), 5.06-4.96 (m, 1H), 4.45-4.24 (m, 2H), 4.03- 3.78 (m, 3H), 3.77-3.66 (m, 3H), 3.64-3.32 (m, 5H), 3.21-2.96 (m, 2H), 2.54-1.85 (m, 8H), 1.54-1.32 (m, 3H). |
| 394a | 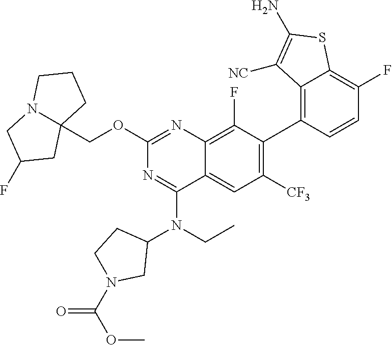 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC)C1] | |
| 395 | 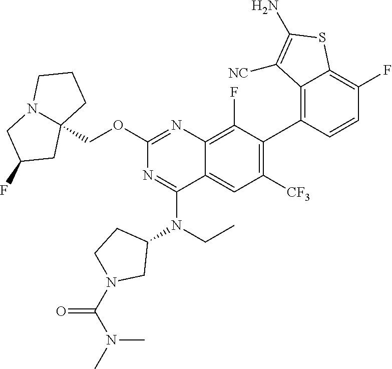 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@H]1CCN(C(=O)N(C)C)C1] | LCMS m/z [M + 1]: 747.2 1H NMR (400 MHz, CD3OD) δ 8.26 (s, 1H), 7.27- 7.17 (m, 1H), 7.01 (t, 1H), 5.45 (s, 0.5H), 5.31 (s, 0.5H), 5.05-4.90 (m, 2H), 4.84-4.67 (m, 1H), 4.48- 4.31 (m, 2H), 3.98-3.76 (m, 3H), 3.71-3.32 (m, 6H), 3.18-3.10 (m, 1H), 2.89 (s, 4H), 2.50-1.89 (m, 8H), 1.45 (s, 3H). |
| 395a | 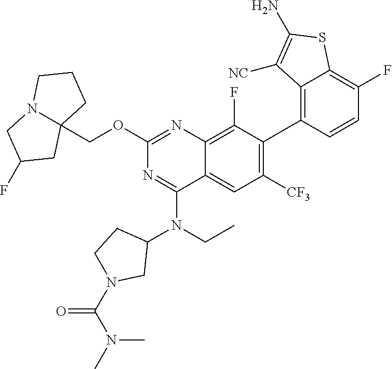 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N(C)C)C1] | |
| 396 | 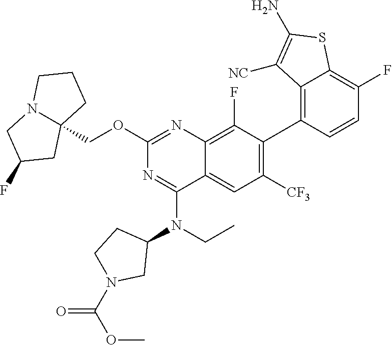 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@@H]1CCN(C(=O)OC)C1] | LCMS m/z [M + 1]: 734.1 1H NMR (400 MHz, CD3OD) δ 8.244 (s, 1H), 7.216 (t, 1H), 7.014 (t, 1H), 5.457 (s, 0.5H), 5.327 (s, 0.5H), 5.008 (t, 1H), 4.482-4.323 (m, 2H), 3.968- 3.801 (m, 3H), 3.719 (s, 3H), 3.631-3.384 (m, 5H), 3.267-3.077 (m, 2H), 2.543-2.299 (m, 4H), 2.244- 1.953 (m, 4H), 1.455 (t, 3H). |
| 397 | 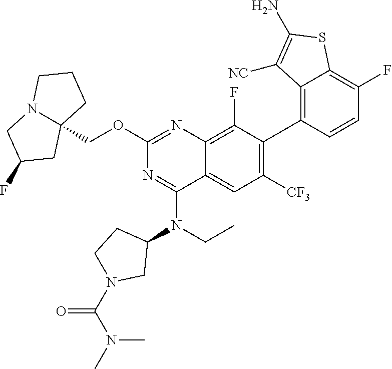 | LCMS m/z [M + 1]: 730.4 1HNMR (400 MHz, CD3OD) δ 8.266 (s, 1H), 7.218 (t, 1H), 7.016 (t, 1H), 5.467 (s, 0.5H), 5.333 (s, 0.5H), 5.007 (t, 1H), 4.483-4.386 (m, 2H), 3.933- 3.810 (m, 3H), 3.641 (t, 3H), 3.553-3.471 (m, 3H), 3.216-3.173 (m, 1H), 2.892 (s, 6H), 2.446-2.311 (m, 4H), 2.239-2.041 (m, 4H), 1.455 (s, 3H). |

TABLE 7
Selected compounds of the present disclosure.

| Cmpd. | | |
|---|---|---|
| No. | Structure | Analytical data |
| 213 | 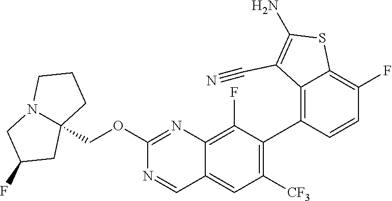 [SMILES: N#Cc1c(N)sc2c(F)ccc(-c3c(C(F)(F)F)cc4cnc(OC[C@@]56CCCN5C[C@H](F)C6)nc4c3F)c12] | LCMS m/z [M + 1]: 567.0 1H NMR (400 MHz, CD3OD) δ 9.60 (s, 1H), 8.41 (s, 1H), 7.21-7.25 (m, 1H), 7.02 (t, 1H), 5.39 (s, 0.5H), 5.25 (s, 0.5H), 4.35-4.45 (m, 2H), 3.22-3.27 (m, 3H), 3.00-3.07 (m, 1H), 2.16-2.42 (m, 4H), 1.99- 2.06 (m, 2H). |
| 213a | 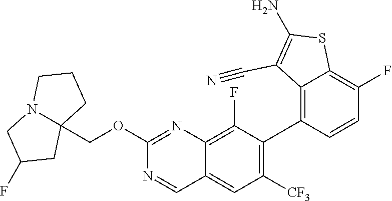 [SMILES: N#Cc1c(N)sc2c(F)ccc(-c3c(C(F)(F)F)cc4cnc(OCC56CCCN5CC(F)C6)nc4c3F)c12] | |
| 318 | 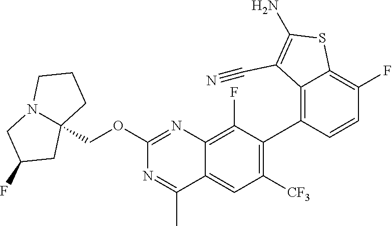 [SMILES: Cc1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 578 (M + H) 1HNMR (400 MHz, CD3OD) δ 8.42 (s, 1H), 7.20- 7.25 (m, 1H), 7.02 (t, 1H), 5.41 (s, 0.5H), 5.28 (s, 0.5H), 4.35-4.47 (m, 2H), 3.32-3.45 (m, 2H), 3.22- 3.27 (m, 1H), 3.05-3.12 (m, 1H), 3.02 (s, 3H), 2.17- 2.42 (m, 3H), 1.92-2.08 (m, 3H). |
| 318a | 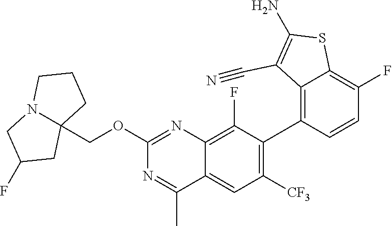 [SMILES: Cc1nc(OCC23CCCN2CC(F)C3)nc2c(F)c(-c3ccc(F)c4sc(N)c(C#N)c34)c(C(F)(F)F)cc12] | |

Table 8 includes selected compounds of the present disclosure.

TABLE 8
Selected compounds of the present disclosure.

| Cmpd. | | |
|---|---|---|
| No. | Structure | Analytical data |
| 398 | 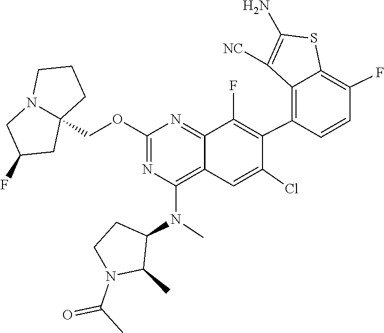 [SMILES: CC(=O)N1CC[C@@H](N(C)c2nc(OC[C@@]34CCCN3C[C@H](F)C4)nc3c(F)c(-c4ccc(F)c5sc(N)c(C#N)c45)c(Cl)cc23)[C@H]1C] | LCMS m/z [M + 1]: 684.3 1H NMR (400 MHz, CD3OD): δ 8.08 (s, 1H), 7.18- 7.24 (m, 1H), 7.01-7.07 (m, 1H), 5.32 (d, J = 54.8 Hz, 1H), 4.92-4.98 (m, 1H), 4.69-4.78 (m, 1H), 4.57- 4.62 (m, 1H), 4.20-4.38 (m, 2H), 3.45-3.70 (m, 5H), 3.1-3.25 (m, 1H), 2.97-3.08 (m, 1H), 2.35-2.65 (m, 3H), 2.20-2.30 (m, 2H), 1.85-2.15 (m, 6H), 1.0- 1.15 (m, 3H). |
| 398a | 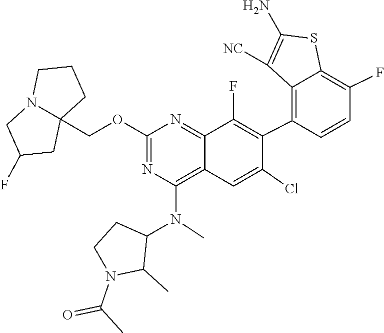 [SMILES: CC(=O)N1CCC(N(C)c2nc(OCC34CCCN3CC(F)C4)nc3c(F)c(-c4ccc(F)c5sc(N)c(C#N)c45)c(Cl)cc23)C1C] | |
| 399 | 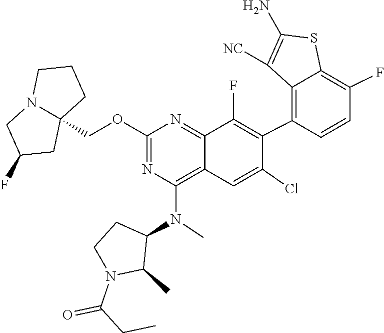 [SMILES: CCC(=O)N1CC[C@@H](N(C)c2nc(OC[C@@]34CCCN3C[C@H](F)C4)nc3c(F)c(-c4ccc(F)c5sc(N)c(C#N)c45)c(Cl)cc23)[C@H]1C] | LCMS m/z [M + 1]: 698.3 1H NMR (400 MHz, CD3OD): δ 8.17 (s, 1H), 7.20- 7.25 (m, 1H), 7.02-7.09 (m, 1H), 5.55 (d, J = 52 Hz, 1H), 4.94-5.00 (m, 1H), 4.55-4.76 (m, 3H), 3.77-4.05 (m, 4H), 3.42-3.64 (m, 4H), 2.54-2.75 (m, 2H), 2.25- 2.50 (m, 5H), 2.05-2.20 (m, 1H), 1.02-1.20 (m, 6H). |
| 399a | 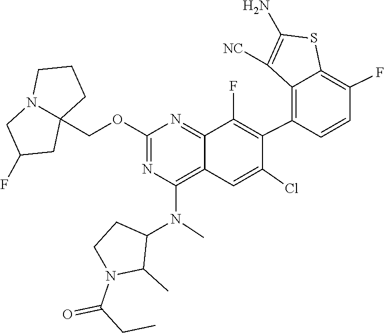 [SMILES: CCC(=O)N1CCC(N(C)c2nc(OCC34CCCN3CC(F)C4)nc3c(F)c(-c4ccc(F)c5sc(N)c(C#N)c45)c(Cl)cc23)C1C] | |
| 400 | 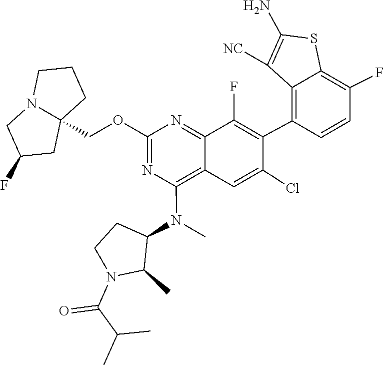 [SMILES: CC(C)C(=O)N1CC[C@@H](N(C)c2nc(OC[C@@]34CCCN3C[C@H](F)C4)nc3c(F)c(-c4ccc(F)c5sc(N)c(C#N)c45)c(Cl)cc23)[C@H]1C] | LCMS m/z [M + 1]: 712.3 1H NMR (400 MHz, CD3OD): δ 8.17 (s, 1H), 7.19- 7.26 (m, 1H), 7.03-7.09 (m, 1H), 5.54 (d, J = 52.4 Hz, 1H), 4.93-5.04 (m, 1H), 4.55-4.80 (m, 3H), 3.80- 4.05 (m, 3H), 3.58-3.75 (m, 3H), 3.41-3.55 (m, 1H), 2.50-2.85 (m, 4H), 2.25-2.47 (m, 3H), 2.04-2.20 (m, 1H), 1.02-1.21 (m, 9H). |
| 400a | 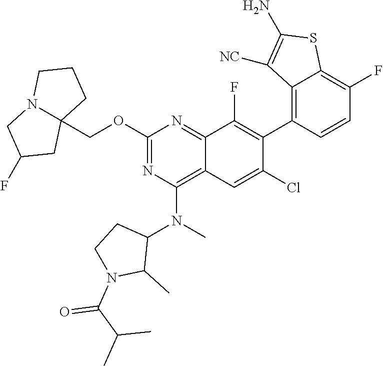 [SMILES: CC(C)C(=O)N1CCC(N(C)c2nc(OCC34CCCN3CC(F)C4)nc3c(F)c(-c4ccc(F)c5sc(N)c(C#N)c45)c(Cl)cc23)C1C] | |
| 401 | 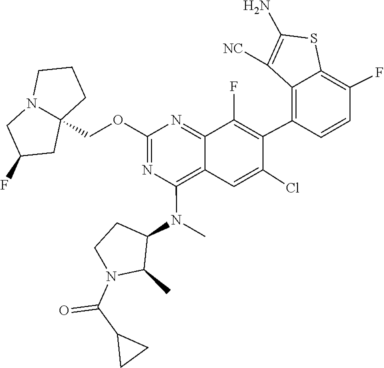 [SMILES: C[C@@H]1[C@H](N(C)c2nc(OC[C@@]34CCCN3C[C@H](F)C4)nc3c(F)c(-c4ccc(F)c5sc(N)c(C#N)c45)c(Cl)cc23)CCN1C(=O)C1CC1] | LCMS m/z [M + 1]: 710.2 1H NMR (400 MHz, CD3OD): δ 8.11 (s, 1H), 7.19- 7.24 (m, 1H), 7.01-7.07 (m, 1H), 5.38 (d, J = 53.2 Hz, 1H), 4.92-5.12 (m, 2H), 4.30-4.80 (m, 3H), 3.98-4.05 (m, 1H), 3.65-3.80 (m, 1H), 3.35-3.62 (m, 6H), 3.05- 3.22 (m, 1H), 1.56-2.55 (m, 8H), 0.55-1.25 (m, 7H). |
| 401a | 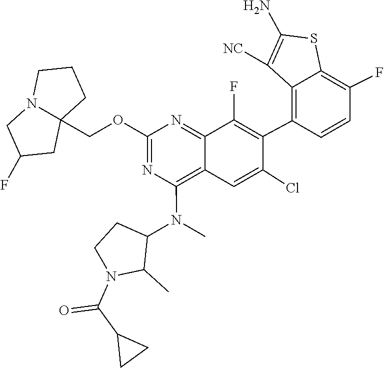 [SMILES: CC1C(N(C)c2nc(OCC34CCCN3CC(F)C4)nc3c(F)c(-c4ccc(F)c5sc(N)c(C#N)c45)c(Cl)cc23)CCN1C(=O)C1CC1] | |
| 402 | 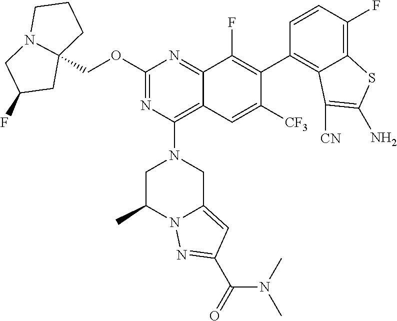 [SMILES: C[C@H]1CN(c2nc(OC[C@@]34CCCN3C[C@H](F)C4)nc3c(F)c(-c4ccc(F)c5sc(N)c(C#N)c45)c(C(F)(F)F)cc23)Cc2cc(C(=O)N(C)C)nn21] | LCMS m/z [M + 1]: 770.2 1HNMR (400 MHz, CD3OD) δ 8.27-8.28 (m, 1H), 7.21-7.24 (m, 1H), 7.03 (t, J = 8.94 Hz, 1H), 6.53 (s, 1H), 5.17-5.40 (m, 3H), 4.73-4.74 (m, 1H), 4.52-4.60 (m, 1H), 4.31-4.43 (m, 2H), 4.06-4.16 (m, 1H), 3.33- 3.48 (m, 6H), 3.06-3.12 (m, 4H), 1.92-2.43 (m, 6H), 1.67-1.70 (m, 3H). |
| 402a | 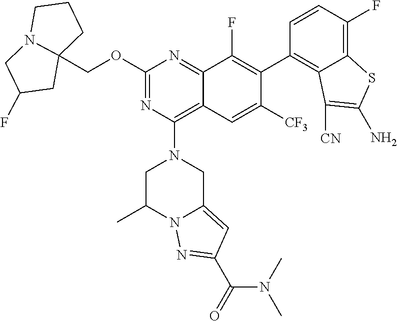 [SMILES: CC1CN(c2nc(OCC34CCCN3CC(F)C4)nc3c(F)c(-c4ccc(F)c5sc(N)c(C#N)c45)c(C(F)(F)F)cc23)Cc2cc(C(=O)N(C)C)nn21] | |
| 403 | 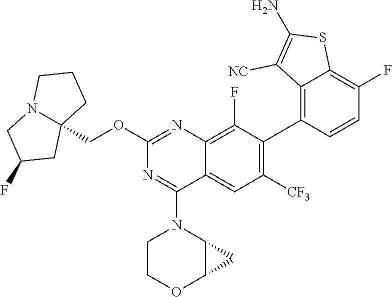 [SMILES: N#Cc1c(N)sc2c(F)ccc(-c3c(C(F)(F)F)cc4c(N5CCO[C@H]6C[C@H]65)nc(OC[C@@]56CCCN5C[C@H](F)C6)nc4c3F)c12] | LCMS m/z [M + 1]: 661.0 1H NMR (400 MHz, CD3OD) δ 9.15 (s, 1H), 7.24- 7.19 (m, 1H), 7.04-6.97 (m, 1H), 5.39 (s, 0.5H), 5.26 (s, 0.5H), 4.39-3.89 (m, 6H), 3.64-3.54 (m, 1H), 3.41-3.32 (m, 2H), 3.29-3.22 (m, 2H), 3.11-3.01 (m, 1H), 2.43-2.13 (m, 3H), 2.07-1.88 (m, 3H), 1.24-1.15 (m, 2H). |
| 404 | 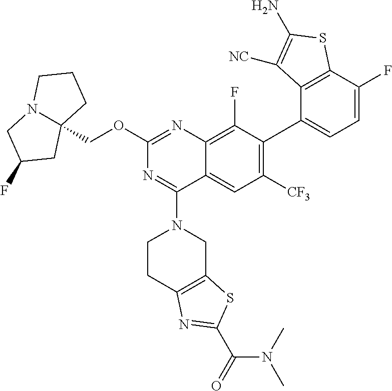 | LCMS m/z [M + 1]: 773.2 1HNMR (400 MHz, CD3OD) δ 8.28 (s, 1H), 7.22- 7.26 (m, 1H), 7.03 (t, J = 9.34 Hz, 1H), 5.56 (d, J = 50.94 Hz, 1H), 5.25-5.34 (m, 2H), 4.61-4.78 (m, 2H), 4.30-4.38 (m, 2H), 3.83-4.08 (m, 3H), 3.57 (s, 3H), 3.43-3.50 (m, 1H), 3.28-3.29 (m, 2H), 3.13 (s, 3H), 2.16-2.77 (m, 6H). |
| 404a | 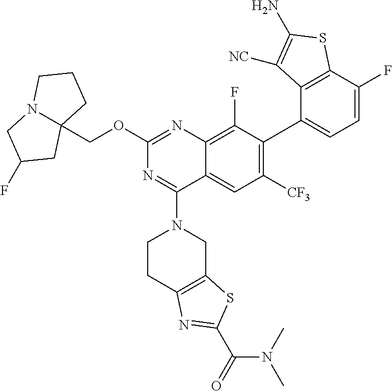 | |
| 405 | 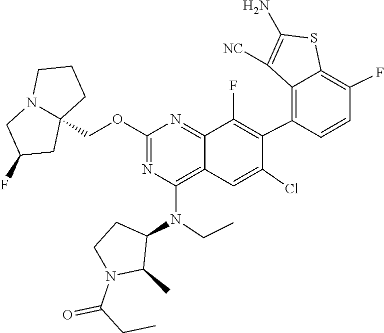 [SMILES: CCC(=O)N1CC[C@@H](N(CC)c2nc(OC[C@@]34CCCN3C[C@H](F)C4)nc3c(F)c(-c4ccc(F)c5sc(N)c(C#N)c45)c(Cl)cc23)[C@H]1C] | LCMS m/z [M + 1]: 746.3 1H NMR (400 MHz, CD3OD) δ 8.37(s, 1H), 7.22- 7.34 (m, 1H), 7.03 (t, J = 8.6 Hz, 1H), 5.56 (d, J = 52.0 Hz, 1H), 4.97-5.05 (m, 1H), 4.65-4.75 (m, 2H), 3.79-4.15 (m, 6H), 3.43-3.72 (m, 2H), 2.31-2.78 (m, 9H), 2.16-2.21 (m, 1H), 1.45-1.50 (m, 3H), 1.09- 1.22 (m, 3H), 0.83-1.0 (m, 3H). |
| 405a | 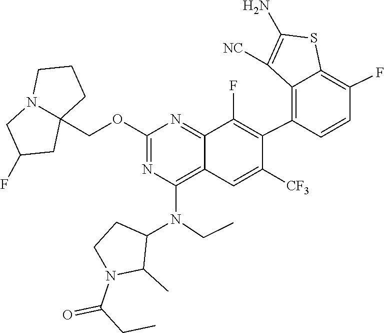 [SMILES: CCC(=O)N1CCC(N(CC)c2nc(OCC34CCCN3CC(F)C4)nc3c(F)c(-c4ccc(F)c5sc(N)c(C#N)c45)c(C(F)(F)F)cc23)C1C] | |
| 406 | 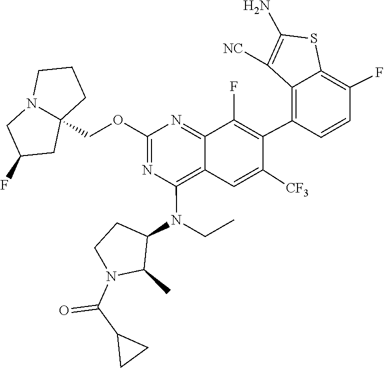 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@@H]1CCN(C(=O)C2CC2)[C@@H]1C] | LCMS m/z [M + 1]: 758.3 1H NMR (400 MHz, CD3OD) δ 8.41 (s, 1H), 7.22- 7.26 (m, 1H), 7.03 (t, J = 8.6 Hz, 1H), 5.56 (d, J = 51.8 Hz, 1H), 4.99-5.09 (m, 1H), 4.60-4.76 (m, 2H), 3.74-4.15 (m, 7H), 3.36-3.49 (m, 2H), 2.33-2.77 (m, 8H), 2.08-2.20 (m, 1H), 1.78-1.85 (m, 1H), 1.44-1.51 (m, 3H), 1.16-1.26 (m, 1H), 0.95-1.05 (m, 2H), 0.86- 0.95 (m, 4H). |
| 406a | 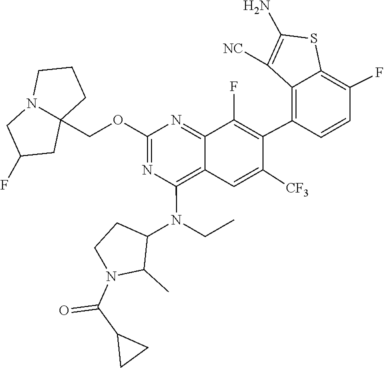 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CC2)C1C] | |
| 407 | 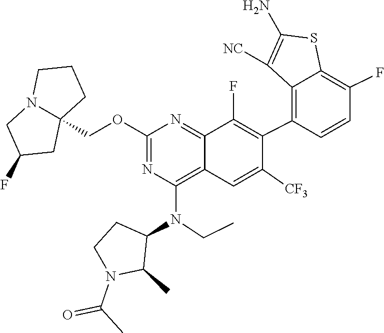 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@@H]1CCN(C(C)=O)[C@@H]1C] | LCMS m/z [M + 1]: 732.3 1H NMR (400 MHz, CD3OD) δ 8.37 (s, 1H), 7.22- 7.25 (m, 1H), 7.05 (t, J = 8.6 Hz, 1H), 5.57 (d, J = 51.8 Hz, 1H), 4.98-5.05 (m, 1H), 4.64-4.74 (m, 3H), 3.34- 4.14 (m, 8H), 2.27-2.74 (m, 8H), 2.08-2.13 (m, 3H), 1.38-1.58 (m, 3H), 0.95-1.12 (m, 3H). |
| 408 | 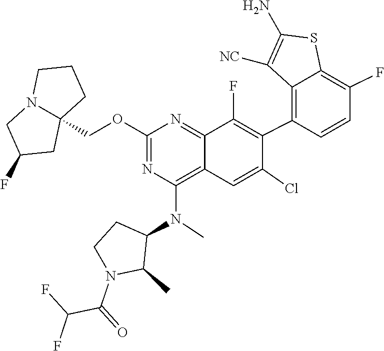 [SMILES: C[C@@H]1[C@H](N(C)c2nc(OC[C@@]34CCCN3C[C@H](F)C4)nc3c(F)c(-c4ccc(F)c5sc(N)c(C#N)c45)c(Cl)cc23)CCN1C(=O)C(F)F] | LCMS m/z [M + 1]: 720.2 1H NMR (400 MHz, CD3OD) δ 8.18 (s, 1H), 7.25- 7.21 (m, 1H), 7.06 (t, J = 8 Hz, 1H), 6.50-6.23 (m, 1H), 5.62-5.49 (m, 1H), 5.12-5.02 (m, 1H), 4.77-4.59 (m, 3H), 4.03-3.44 (m, 9H), 2.75-2.03 (m, 8H), 1.18- 1.08 (m, 3H). |
| 408a | 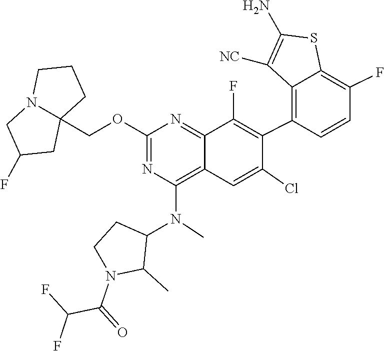 [SMILES: CC1C(N(C)c2nc(OCC34CCCN3CC(F)C4)nc3c(F)c(-c4ccc(F)c5sc(N)c(C#N)c45)c(Cl)cc23)CCN1C(=O)C(F)F] | |
| 409 | 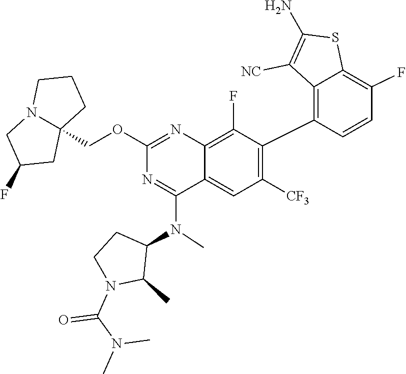 [SMILES: C[C@@H]1[C@H](N(C)c2nc(OC[C@@]34CCCN3C[C@H](F)C4)nc3c(F)c(-c4ccc(F)c5sc(N)c(C#N)c45)c(C(F)(F)F)cc23)CCN1C(=O)N(C)C] | LCMS m/z [M + 1]: 747.1 1H NMR (400 MHz, CD3OD) δ 8.33 (s, 1H), 7.19- 7.23 (m, 1H), 7.01 (t, 1H), 5.38 (s, 0.5H), 5.25-5.30 (m, 1.5H), 4.67-4.71 (m, 1H), 4.24-4.37 (m, 2H), 3.86-3.82 (m, 1H), 3.58 (s, 3H), 3.32-3.35 (m, 1H), 3.17-3.26 (m, 3H), 3.00-3.04 (m, 1H), 2.91 (s, 6H), 2.13-2.50 (m, 5H), 1.89-2.04 (m, 3H), 1.13 (t, 3H). |
| 409a | 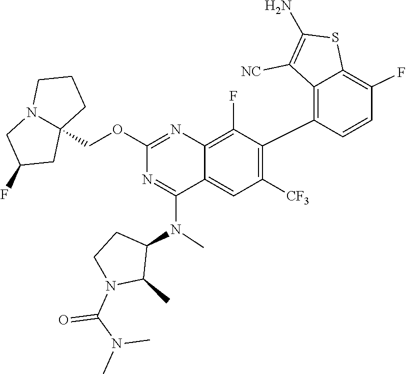 [SMILES: C[C@@H]1[C@H](N(C)c2nc(OC[C@@]34CCCN3C[C@H](F)C4)nc3c(F)c(-c4ccc(F)c5sc(N)c(C#N)c45)c(C(F)(F)F)cc23)CCN1C(=O)N(C)C] | |
| 410 | 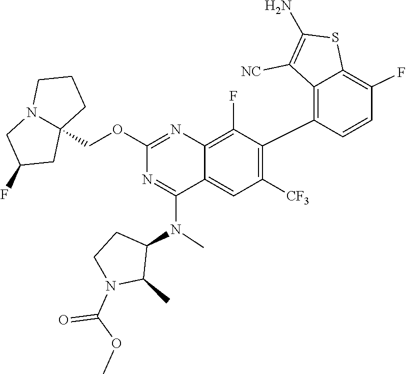 [SMILES: COC(=O)N1CC[C@@H](N(C)c2nc(OC[C@@]34CCCN3C[C@H](F)C4)nc3c(F)c(-c4ccc(F)c5sc(N)c(C#N)c45)c(C(F)(F)F)cc23)[C@H]1C] | LCMS m/z [M + 1]: 734.1 1H NMR (400 MHz, CD3OD) δ 8.33 (d, 1H), 7.20- 7.23 (m, 1H), 7.01 (t, 1H), 5.37 (s, 0.5H), 5.24 (s, 0.5H), 4.72-4.76 (m, 1H), 4.21-4.35 (m, 2H), 3.72 (s, 3H), 3.63-3.67 (m, 1H), 3.58 (s, 3H), 3.40-3.47 (m, 1H), 3.20-3.25 (m, 3H), 2.99-3.05 (m, 1H), 2.52-2.59 (m, 1H), 1.90-2.38 (m, 8H), 1.04-1.09 (m, 3H). |
| 410a | 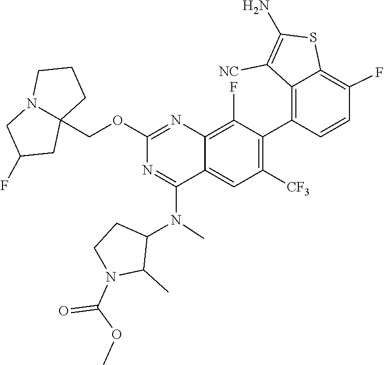 [SMILES: COC(=O)N1CCC(N(C)c2nc(OCC34CCCN3CC(F)C4)nc3c(F)c(-c4ccc(F)c5sc(N)c(C#N)c45)c(C(F)(F)F)cc23)C1C] | |
| 411 | 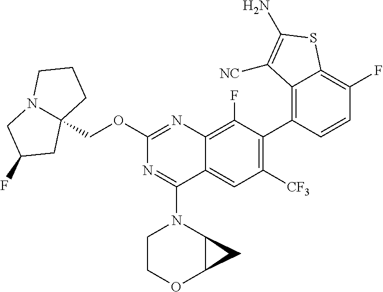 [SMILES: N#Cc1c(N)sc2c(F)ccc(-c3c(C(F)(F)F)cc4c(N5CCO[C@@H]6C[C@@H]65)nc(OC[C@@]56CCCN5C[C@H](F)C6)nc4c3F)c12] | LCMS m/z [M + 1]: 661.3 1H NMR (400 MHz, CD3OD) δ 9.16 (s, 1H), 7.27- 7.17 (m, 1H), 7.05-6.95 (m, 1H), 5.43 (s, 0.5H), 5.29 (s, 0.5H), 4.46-3.91 (m, 6H), 3.63-3.56 (m, 1H), 3.52- 3.43 (m, 1H), 3.42-3.33 (m, 2H), 3.22-3.06 (m, 2H), 2.43-2.16 (m, 3H), 2.13-1.87 (m, 3H), 1.27-1.12 (m, 2H) |
| 412 | 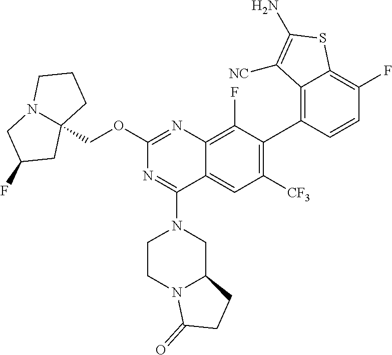 | LCMS m/z [M + 1]: 702.20 1H NMR (400 MHz, CD3OD) δ 8.13 (s, 1H), 7.24- 7.18 (m, 1H), 7.04-6.97 (m, 1H), 5.37 (s, 0.5H), 5.23 (s, 0.5H), 4.67-4.58 (m, 1H), 4.57-4.48 (m, 1H), 4.37-4.30 (m, 1H), 4.28-4.22 (m, 1H), 4.14-4.04 (m, 1H), 4.00-3.87 (m, 1H), 3.40-3.32 (m, 1H), 3.28-3.11 (m, 5H), 3.07-2.96 (m, 1H), 2.54-2.40 (m, 2H), 2.35- 2.11 (m, 4H), 2.04-1.85 (m, 3H), 1.82-1.71 (m, 1H). |
| 412a | 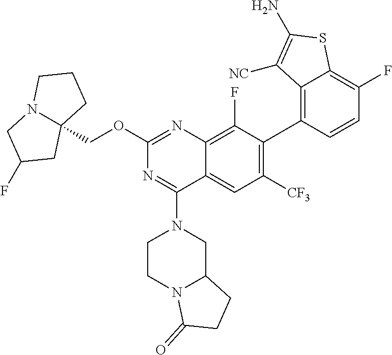 | |
| 413 | 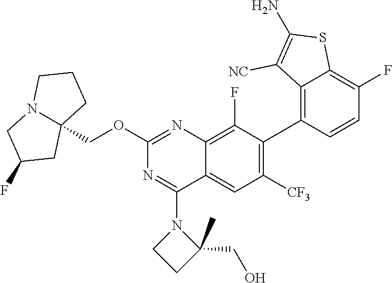 [SMILES: C[C@@]1(CO)CCN1c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 663.0 1H NMR (400 MHz, CD3OD) δ 8.18 (s, 1H), 7.22- 7.19 (m, 1H), 7.04-6.99 (m, 1H), 5.58 (s, 0.5H), 5.45 (s, 0.5H), 4.71-4.58 (m, 4H), 4.33-4.30 (m, 1H), 3.86-3.82 (m, 3H), 3.67-3.64 (m, 1H), 3.47-3.43 (m, 1H), 2.71-2.54 (m, 3H), 2.38-2.27 (m, 4H), 2.11-2.03 (m, 1H), 1.74 (s, 3H). |
| 413a | 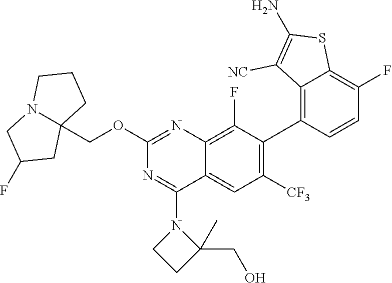 [SMILES: CC1(CO)CCN1c1nc(OCC23CCCN2CC(F)C3)nc2c(F)c(-c3ccc(F)c4sc(N)c(C#N)c34)c(C(F)(F)F)cc12] | |
| 414 | 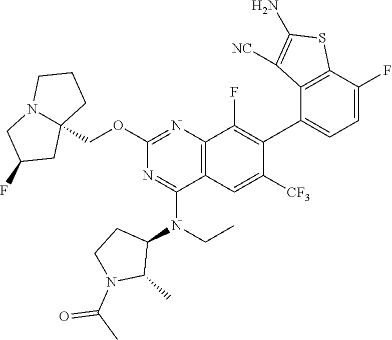 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@@H]1CCN(C(C)=O)[C@H]1C] | LCMS m/z [M + 1]: 732.3 1HNMR (400 MHz, CD3OD) δ 8.22-8.27 (m, 1H), 7.21-7.28 (m, 1H), 7.03 (t, J = 9.2 Hz, 1H), 5.49-5.67 (m, 1H), 4.15-4.70 (m, 4H), 3.64-4.05 (m, 6H), 3.40- 3.48 (m, 1H), 2.30-2.72 (m, 8H), 2.05-2.20 (m, 4H), 1.32-1.55 (m, 6H). |
| 415 | 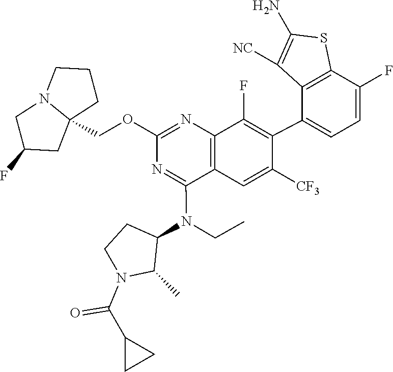 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@@H]1CCN(C(=O)C2CC2)[C@H]1C] | LCMS m/z [M + 1]: 758.3 1HNMR (400 MHz, CD3OD) δ 8.26 (s, 1H), 7.22- 7.26 (m, 1H), 7.03 (t, J = 9.2 Hz, 1H), 5.56 (d, J = 51.4 Hz, 1H), 4.46-4.71 (m, 4H), 3.78-4.22 (m, 7H), 3.40-3.48 (m, 1H), 2.31-2.72 (m, 7H), 2.06-2.16 (m, 1H), 1.85-1.90 (m, 1H), 1.44-1.54 (m, 4H), 1.30-1.36 (m, 2H), 0.79-0.94 (m, 4H). |
| 416 | 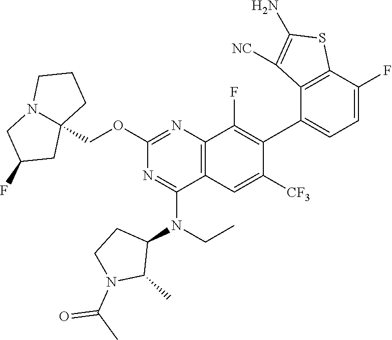 | LCMS m/z [M + 1]: 698.2 1H NMR (400 MHz, CD3OD) δ 7.93-8.06 (m, 1H), 7.21-7.25 (m, 1H), 7.06 (t, J = 8.9 Hz, 1H), 5.45-5.69 (m, 1H), 4.34-4.74 (m, 4H), 3.63-4.24 (m, 7H), 3.38- 3.49 (m, 1H), 2.26-2.79 (m, 7H), 2.05-2.22 (m, 4H), 1.38-1.48 (m, 4H). 1.33 (dd, J = 13.1, 6.3 Hz, 1H). |
| 417 | 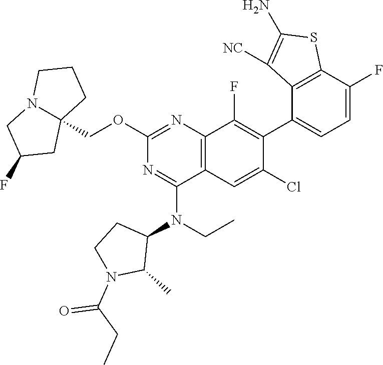 [SMILES: CCC(=O)N1CC[C@@H](N(CC)c2nc(OC[C@@]34CCCN3C[C@H](F)C4)nc3c(F)c(-c4ccc(F)c5sc(N)c(C#N)c45)c(Cl)cc23)[C@@H]1C] | LCMS m/z [M + 1]: 712.3 1H NMR (400 MHz, CD3OD) δ 7.94-8.01 (m, 1H), 7.20-7.26 (m, 1H), 7.06 (t, J = 8.9 Hz, 1H), 5.56 (d, J = 51.9 Hz, 1H), 4.32-4.74 (m, 4H), 3.59-4.20 (m, 7H), 3.41-3.50 (m, 1H), 2.26-2.77 (m, 9H), 2.13 (s, 1H), 1.23-1.49 (m, 6H), 1.08-1.18 (m, 3H). |
| 418 | 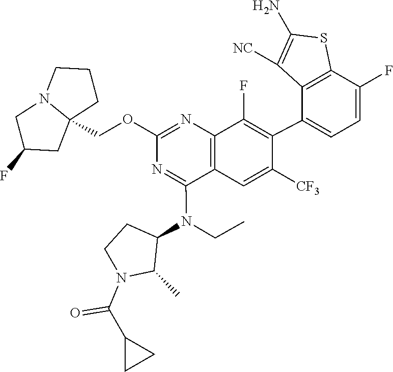 | LCMS m/z [M + 1]: 724.3 1H NMR (400 MHz, CD3OD) δ 7.93-8.07 (m, 1H), 7.20-7.26 (m, 1H), 7.06 (dd, J = 9.5, 8.4 Hz, 1H), 5.56 (d, J = 52.2 Hz, 1H), 4.36-4.74 (m, 4H), 3.72- 4.22 (m, 7H), 3.39-3.54 (m, 1H), 2.25-2.77 (m, 7H), 2.14 (s, 1H), 1.82-1.94 (m, 1H), 1.26-1.57 (m, 6H), 0.79-1.54 (m, 4H). |
| 419 | 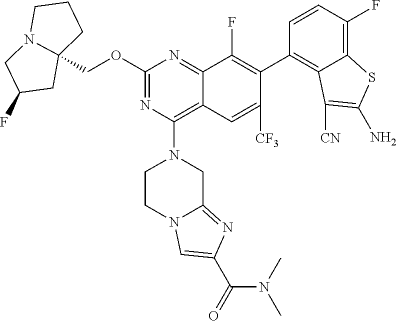 [SMILES: CN(C)C(=O)c1cn2c(n1)CN(c1nc(OC[C@@]34CCCN3C[C@H](F)C4)nc3c(F)c(-c4ccc(F)c5sc(N)c(C#N)c45)c(C(F)(F)F)cc13)CC2] | LCMS m/z [M + 1]: 756.2 1HNMR (400 MHz, CD3OD) δ 8.31 (s, 1H), 7.81 (s, 1H), 7.22-7.25 (m, 1H), 7.03 (t, J = 9.28 Hz, 1H), 5.57 (d, J = 51.44 Hz, 1H), 5.30-5.40 (m, 2H), 4.65- 4.78 (m, 2H), 4.43-4.55 (m, 4H), 3.89-4.06 (m, 3H), 3.44-3.50 (m, 1H), 3.34 (s, 3H), 3.09 (s, 3H), 2.16- 2.76 (m, 6H). |
| 419a | 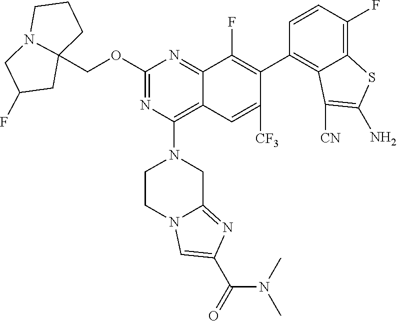 [SMILES: CN(C)C(=O)c1cn2c(n1)CN(c1nc(OCC34CCCN3CC(F)C4)nc3c(F)c(-c4ccc(F)c5sc(N)c(C#N)c45)c(C(F)(F)F)cc13)CC2] | |
| 420 | 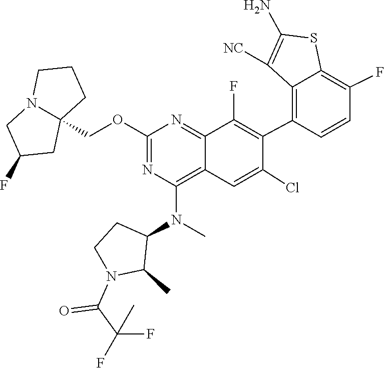 [SMILES: C[C@@H]1[C@H](N(C)c2nc(OC[C@@]34CCCN3C[C@H](F)C4)nc3c(F)c(-c4ccc(F)c5sc(N)c(C#N)c45)c(Cl)cc23)CCN1C(=O)C(C)(F)F] | LCMS m/z [M + 1]: 734.2 1HNMR (400 MHz, CD3OD) δ 8.09-8.11 (m, 1H), 7.20-7.23 (m, 1H), 7.04 (t, J = 9.04 Hz, 1H), 5.01- 5.46 (m, 2H), 4.69-4.75 (m, 1H), 4.28-4.48 (m, 2H), 3.78-4.11 (m, 1H), 3.41-3.70 (m, 7H), 3.12-3.17 (m, 1H), 1.97-2.64 (m, 8H), 1.77-1.91 (m, 3H), 1.04-1.14 (m, 3H). |
| 420a | 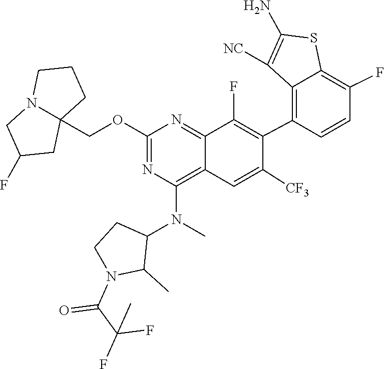 [SMILES: CC1C(N(C)c2nc(OCC34CCCN3CC(F)C4)nc3c(F)c(-c4ccc(F)c5sc(N)c(C#N)c45)c(C(F)(F)F)cc23)CCN1C(=O)C(C)(F)F] | |
| 421 | 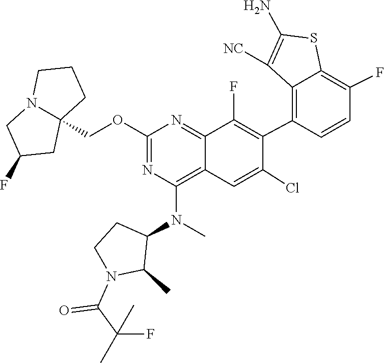 [SMILES: C[C@@H]1[C@H](N(C)c2nc(OC[C@@]34CCCN3C[C@H](F)C4)nc3c(F)c(-c4ccc(F)c5sc(N)c(C#N)c45)c(Cl)cc23)CCN1C(=O)C(C)(C)F] | LCMS m/z [M + 1]: 730.3 1HNMR (400 MHz, CD3OD) δ 8.07-8.09 (m, 1H), 7.19-7.23 (m, 1H), 7.03 (t, J = 8.88 Hz, 1H), 5.02- 5.43 (m, 2H), 4.67-4.73 (m, 1H), 4.23-4.43 (m, 2H), 3.80-4.12 (m, 1H), 3.35-3.67 (m, 7H), 3.09-3.12 (m, 1H), 1.93-2.64 (m, 8H), 1.56-1.69 (m, 6H), 1.02-1.13 (m, 3H). |
| 421a | 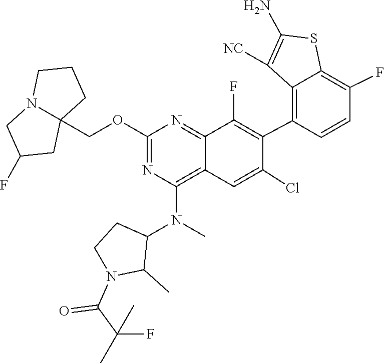 [SMILES: CC1C(N(C)c2nc(OCC34CCCN3CC(F)C4)nc3c(F)c(-c4ccc(F)c5sc(N)c(C#N)c45)c(Cl)cc23)CCN1C(=O)C(C)(C)F] | |
| 422 | 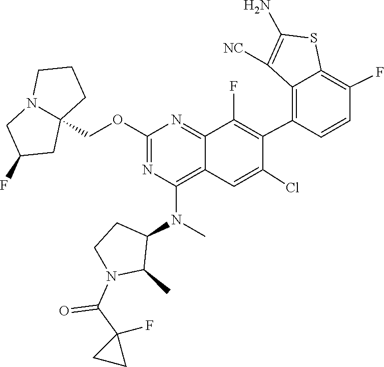 [SMILES: C[C@@H]1[C@H](N(C)c2nc(OC[C@@]34CCCN3C[C@H](F)C4)nc3c(F)c(-c4ccc(F)c5sc(N)c(C#N)c45)c(Cl)cc23)CCN1C(=O)C1(F)CC1] | LCMS m/z [M + 1]: 728.2 1HNMR (400 MHz, CD3OD) δ 8.30 (s, 1H), 7.25- 7.28 (m, 1H), 7.07 (t, J = 9.08 Hz, 1H), 5.56 (d, J = 52.04 Hz, 1H), 4.97-5.08 (m, 2H), 4.75-4.77 (m, 2H), 3.89-4.17 (m, 4H), 3.74 (s, 3H), 3.47-3.51 (m, 1H), 2.18-2.78 (m, 8H), 1.13-1.35 (m, 8H). |
| 422a | 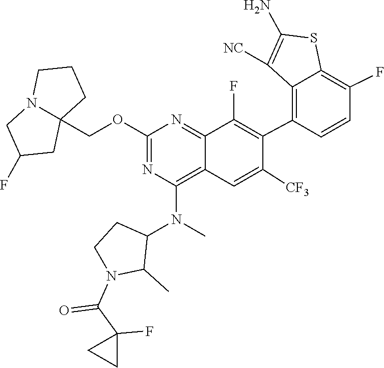 [SMILES: CC1C(N(C)c2nc(OCC34CCCN3CC(F)C4)nc3c(F)c(-c4ccc(F)c5sc(N)c(C#N)c45)c(C(F)(F)F)cc23)CCN1C(=O)C1(F)CC1] | |
| 423 | 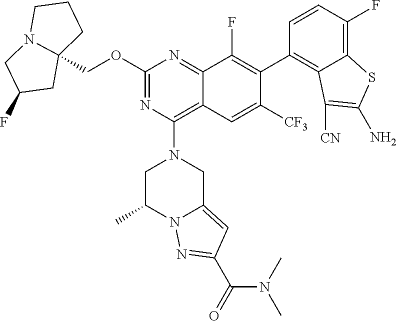 [SMILES: C[C@@H]1CN(c2nc(OC[C@@]34CCCN3C[C@H](F)C4)nc3c(F)c(-c4ccc(F)c5sc(N)c(C#N)c45)c(C(F)(F)F)cc23)Cc2cc(C(=O)N(C)C)nn21] | LCMS m/z [M + 1]: 770.2 1HNMR (400 MHz, CD3OD) δ 8.33 (s, 1H), 7.23- 7.26 (m, 1H), 7.03 (t, J = 8.98 Hz, 1H), 6.54 (d, J = 1.80 Hz, 1H), 5.57 (d, J = 51.80 Hz, 1H), 5.23-5.39 (m, 2H), 4.58-4.78 (m, 4H), 3.84-4.23 (m, 4H), 3.44-3.51 (m, 1H), 3.34 (s, 3H), 3.10 (s, 3H), 2.17-2.76 (m, 6H), 1.68-1.70 (m, 3H). |
| 424 | 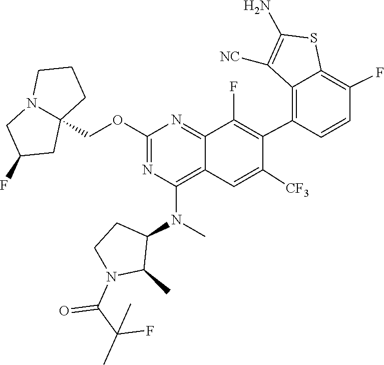 [SMILES: C[C@@H]1[C@H](N(C)c2nc(OC[C@@]34CCCN3C[C@H](F)C4)nc3c(F)c(-c4ccc(F)c5sc(N)c(C#N)c45)c(C(F)(F)F)cc23)CCN1C(=O)C(C)(C)F] | LCMS m/z [M + 1]: 764.3 1HNMR (400 MHz, CD3OD) δ 8.41 (s, 1H), 7.22- 7.26 (m, 1H), 7.03 (t, J = 8.9 Hz, 1H), 5.56 (d, J = 51.9 Hz, 1H), 5.02-5.10 (m, 1H), 4.57-4.81 (m, 3H), 4.0- 4.15 (m, 1H), 3.80-3.95 (m, 3H), 3.62-3.74 (m, 4H), 3.40-3.50 (m, 1H), 2.05-2.74 (m, 8H), 1.55-1.70 (m, 6H), 1.05-1.20 (m, 3H). |
| 424a | 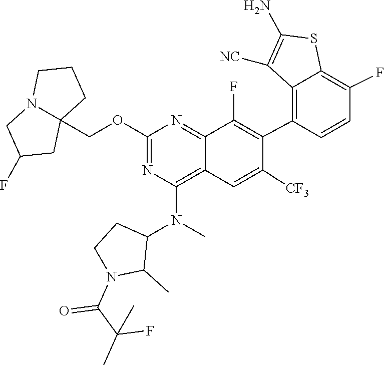 [SMILES: CC1C(N(C)c2nc(OCC34CCCN3CC(F)C4)nc3c(F)c(-c4ccc(F)c5sc(N)c(C#N)c45)c(C(F)(F)F)cc23)CCN1C(=O)C(C)(C)F] | |
| 425 | 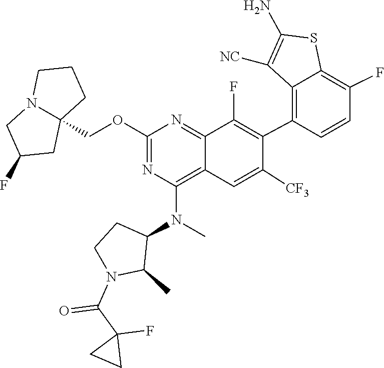 [SMILES: C[C@@H]1[C@H](N(C)c2nc(OC[C@@]34CCCN3C[C@H](F)C4)nc3c(F)c(-c4ccc(F)c5sc(N)c(C#N)c45)c(C(F)(F)F)cc23)CCN1C(=O)C1(F)CC1] | LCMS m/z [M + 1]: 762.2 1HNMR (400 MHz, CD3OD) δ 8.42 (s, 1H), 7.21- 7.27 (m, 1H), 7.03 (d, J = 11.2 Hz, 1H), 5.56 (d, J = 52.6 Hz, 1H), 5.02-5.15 (m, 1H), 4.58-4.79 (m, 2H), 3.82-4.17 (m, 5H), 3.42-3.70 (m, 4H), 2.27-2.80 (m, 8H), 2.07-2.20 (m, 1H), 1.07-1.45 (m, 7H). |
| 425a | 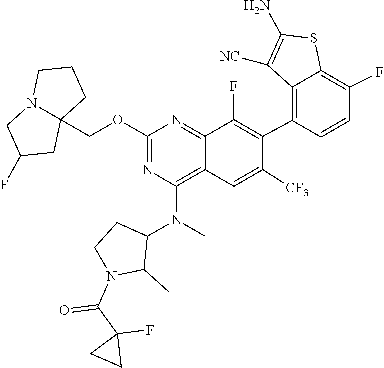 [SMILES: CC1C(N(C)c2nc(OCC34CCCN3CC(F)C4)nc3c(F)c(-c4ccc(F)c5sc(N)c(C#N)c45)c(C(F)(F)F)cc23)CCN1C(=O)C1(F)CC1] | |
| 426 | 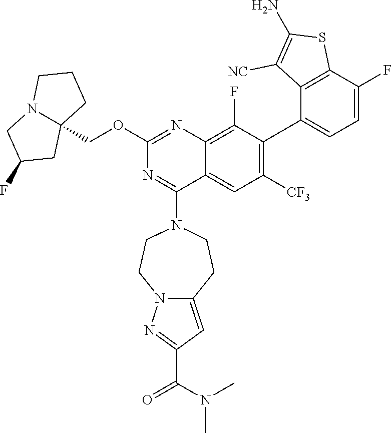 [SMILES: CN(C)C(=O)c1cc2n(n1)CCN(c1nc(OC[C@@]34CCCN3C[C@H](F)C4)nc3c(F)c(-c4ccc(F)c5sc(N)c(C#N)c45)c(C(F)(F)F)cc13)CC2] | LCMS m/z [M + 1]: 770.2 1H NMR (400 MHz, CD3OD) δ 8.27 (s, 1H), 7.21- 7.25 (m, 1H), 7.03 (t, J = 9.28 Hz, 1H), 6.48 (s, 1H), 5.57 (d, J = 52 Hz, 1H), 4.63-4.81 (m, 4H), 4.27-4.42 (m, 4H), 3.87-4.06 (m, 3H), 3.37-3.48 (m, 3H), 3.07 (s, 3H), 2.17-2.77 (m, 6H). |
| 426a | 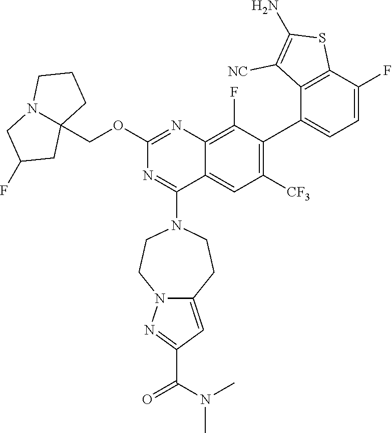 [SMILES: CN(C)C(=O)c1cc2n(n1)CCN(c1nc(OCC34CCCN3CC(F)C4)nc3c(F)c(-c4ccc(F)c5sc(N)c(C#N)c45)c(C(F)(F)F)cc13)CC2] | |
| 427 | 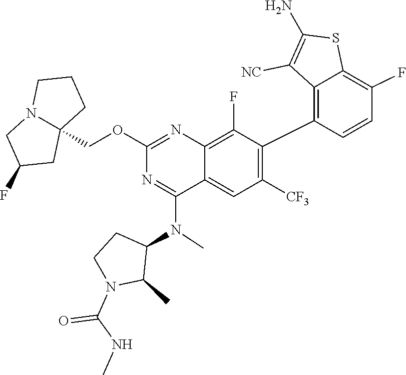 [SMILES: CNC(=O)N1CC[C@@H](N(C)c2nc(OC[C@@]34CCCN3C[C@H](F)C4)nc3c(F)c(-c4ccc(F)c5sc(N)c(C#N)c45)c(C(F)(F)F)cc23)[C@H]1C] | LCMS m/z [M + 1]: 733.1 1H NMR (400 MHz, CD3OD) δ 8.35 (s, 1H), 7.20- 7.23 (m, 1H), 7.01 (t, 1H), 5.41 (s, 0.5H), 5.28 (s, 0.5H), 4.81-4.83 (m, 1H), 4.73-4.76 (m, 1H), 4.29- 4.42 (m, 2H), 3.58-3.60 (m, 3H), 3.36-3.47 (m, 4H), 3.06-3.12 (m, 1H), 2.75 (s, 3H), 2.61-2.64 (m, 1H), 1.91-2.45 (m, 8H), 1.03-1.07 (m, 3H). |
| 427a | 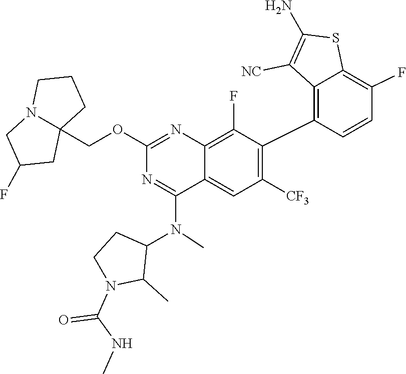 [SMILES: CNC(=O)N1CCC(N(C)c2nc(OCC34CCCN3CC(F)C4)nc3c(F)c(-c4ccc(F)c5sc(N)c(C#N)c45)c(C(F)(F)F)cc23)C1C] | |
| 428 | 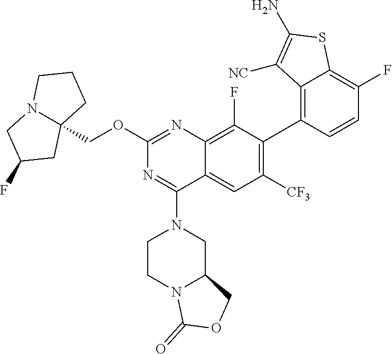 | LCMS m/z [M + 1]: 704.3 1H NMR (400 MHz, DMSO) δ 8.09 (d, J = 4.8 Hz, 1H), 7.26-7.22 (m, 1H), 7.17-7.12 (m, 1H), 5.34 (s, 0.55H), 5.21 (s, 0.57H), 4.53-4.04 (m, 8H), 3.80-3.74 (m, 1H), 3.10-3.01 (m, 3H), 2.83-2.81 (m, 1H), 2.67 (s, 1H), 2.33 (s, 1H), 2.14-2.01 (m, 3H), 1.85-1.77 (m, 3H). |
| 428a | 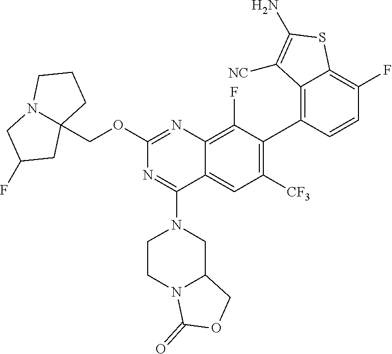 | |
| 429 | 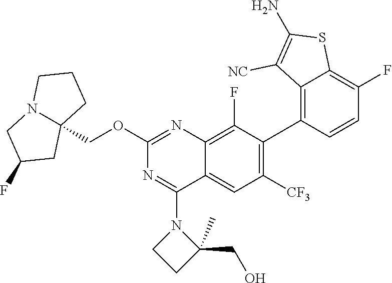 [SMILES: C[C@]1(CO)CCN1c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 663.3 1H NMR (400 MHz, CD3OD) δ 8.12 (d, J = 28.8 Hz, 1H), 7.19 (d, J = 5.2 Hz, 1H), 7.03-6.99 (m, 1H), 5.51 (s, 0.56H), 5.34 (s, 1H), 5.18 (s, 0.54H), 4.78-4.32 (m, 4H), 3.66-3.40 (m, 4H), 3.13 (s, 1H), 2.70 (s, 1H), 2.42-2.17 (m, 5H), 2.01 (s, 2H), 1.70-1.60 (m, 3H). |
| 430 | 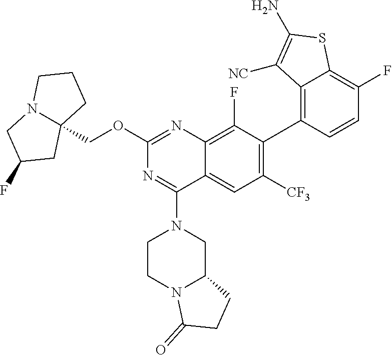 | LCMS m/z [M + 1]: 702.20 1H NMR (400 MHz, CD3OD) δ 8.19 (s, 1H), 7.25- 7.19 (m, 1H), 7.03 (t, J = 9.2 Hz, 1H), 5.60 (s, 0.5H), 5.47 (s, 0.5H), 4.73-4.56 (m, 4H), 4.13-4.08 (d, J = 6.4 Hz, 1H), 3.97-3.92 (m, 1H), 3.87-3.79 (m, 2H), 3.48-3.39 (m, 2H), 3.25-3.09 (m, 3H), 2.73-2.64 (m, 1H), 2.60-2.53 (m, 1H), 2.51-2.44 (m, 2H), 2.43-2.37 (m, 1H), 2.36-2.25 (m, 3H), 2.17-2.07 (m, 1H), 1.84- 1.74 (m, 1H). |
| 431 | 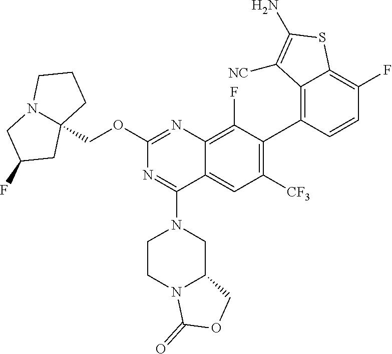 | LCMS m/z [M + 1]: 704.0 1H NMR (400 MHz, CD3OD) δ 8.14 (s, 1H), 7.23- 7.20 (m, 1H), 7.04-7.00 (m, 1H), 5.37 (s, 0.5H), 5.23 (s, 0.5H), 4.64-4.45 (m, 3H), 4.24-4.13 (m, 4H), 3.88- 3.86 (m, 1H), 3.43-3.31 (m, 3H), 3.30-3.13 (m, 3H), 3.00-2.98 (m, 1H), 2.37-2.13 (m, 3H), 2.01-1.88 (m, 3H). |
| 432 | 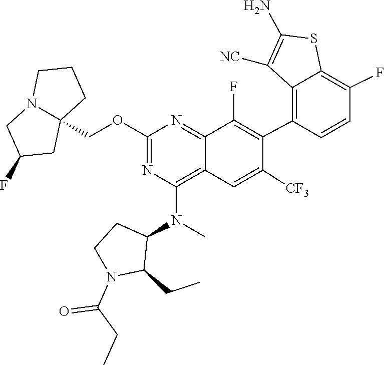 [SMILES: CCC(=O)N1CC[C@@H](N(C)c2nc(OC[C@@]34CCCN3C[C@H](F)C4)nc3c(F)c(-c4ccc(F)c5sc(N)c(C#N)c45)c(C(F)(F)F)cc23)[C@H]1CC] | LCMS m/z [M + 1]: 746.3 1HNMR (400 MHz, CD3OD): δ 8.41 (s, 1H), 7.20- 7.26 (m, 1H), 7.00-7.06 (m, 1H), 5.56 (d, J = 52.4 Hz, 1H), 4.99-5.07 (m, 1H), 4.58-4.78 (m, 3H), 375- 4.06 (m, 4H), 3.56-3.69 (m, 4H), 3.42-3.52 (m, 1H), 2.57-2.77 (m, 3H), 2.28-2.48 (m, 6H), 2.06-2.25 (m, 1H), 1.46-1.58 (m, 2H), 1.11-1.21 (m, 3H), 0.82- 0.88 (m, 3H). |
| 432a | 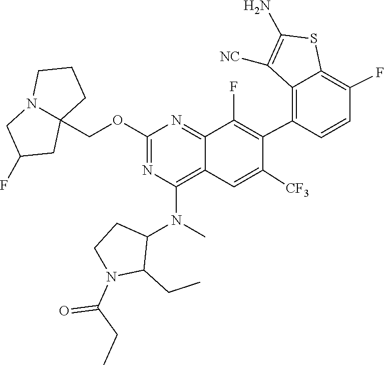 [SMILES: CCC(=O)N1CCC(N(C)c2nc(OCC34CCCN3CC(F)C4)nc3c(F)c(-c4ccc(F)c5sc(N)c(C#N)c45)c(C(F)(F)F)cc23)C1CC] | |
| 433 | 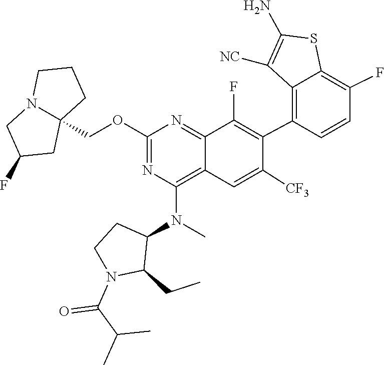 [SMILES: CC[C@@H]1[C@H](N(C)c2nc(OC[C@@]34CCCN3C[C@H](F)C4)nc3c(F)c(-c4ccc(F)c5sc(N)c(C#N)c45)c(C(F)(F)F)cc23)CCN1C(=O)C(C)C] | LCMS m/z [M + 1]: 760.3 1HNMR (400 MHz, CD3OD): δ 8.41 (s, 1H), 7.20- 7.27 (m, 1H), 7.00-7.06 (m, 1H), 5.56 (d, J = 52.0 Hz, 1H), 5.02-5.10 (m, 1H), 4.58-4.77 (m, 3H), 3.80- 4.08 (m, 4H), 3.55-3.77 (m, 4H), 3.42-3.52 (m, 1H), 2.25-2.90 (m, 8H), 2.08-2.24 (m, 1H), 1.47-1.58 (m, 2H), 1.11-1.21 (m, 6H), 0.81-0.87 (m, 3H) |
| 433a | 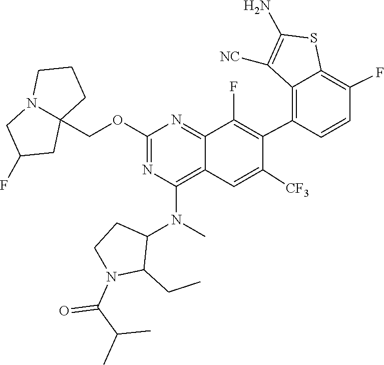 [SMILES: CCC1C(N(C)c2nc(OCC34CCCN3CC(F)C4)nc3c(F)c(-c4ccc(F)c5sc(N)c(C#N)c45)c(C(F)(F)F)cc23)CCN1C(=O)C(C)C] | |
| 434 | 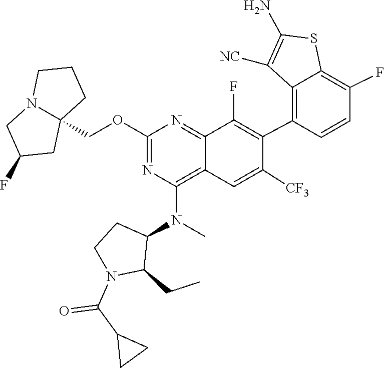 [SMILES: CC[C@@H]1[C@H](N(C)c2nc(OC[C@@]34CCCN3C[C@H](F)C4)nc3c(F)c(-c4ccc(F)c5sc(N)c(C#N)c45)c(C(F)(F)F)cc23)CCN1C(=O)C1CC1] | LCMS m/z [M + 1]: 758.3 1HNMR (400 MHz, CD3OD): δ 8.40-8.45 (m, 1H), 7.20-7.26 (m, 1H), 7.00-7.06 (m, 1H), 5.56 (d, J = 53.2 Hz, 1H), 4.94-5.06 (m, 1H), 4.57-4.79 (m, 3H), 3.82-4.04 (m, 4H), 3.57-3.70 (m, 4H), 3.41-3.51 (m, 1H), 2.05-2.80 (m, 8H), 1.83-1.93 (m, 1H), 1.49-1.77 (m, 2H), 0.80-1.02 (m, 7H). |
| 434a | 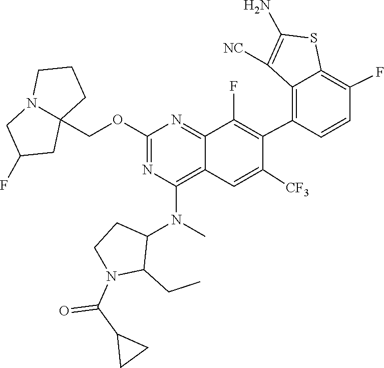 [SMILES: CCC1C(N(C)c2nc(OCC34CCCN3CC(F)C4)nc3c(F)c(-c4ccc(F)c5sc(N)c(C#N)c45)c(C(F)(F)F)cc23)CCN1C(=O)C1CC1] | |
| 435 | 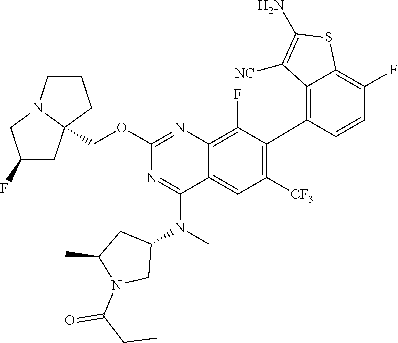 [SMILES: CCC(=O)N1C[C@@H](N(C)c2nc(OC[C@@]34CCCN3C[C@H](F)C4)nc3c(F)c(-c4ccc(F)c5sc(N)c(C#N)c45)c(C(F)(F)F)cc23)C[C@@H]1C] | LCMS m/z [M + 1]: 732.1 1H NMR (400 MHz, CD3OD) δ 8.28 (d, 1H), 7.23 (m, 1H), 7.03 (m, 1H), 6.81-6.88 (m, 0.5H), 6.60- 6.68 (m, 0.5H), 6.33 (d, 1H), 5.98-6.06 (m, 1H), 5.50-5.83 (m, 1H), 5.40 (s, 0.5H), 5.27 (s, 0.5H), 4.50-4.60 (m, 1H), 3.84-3.92 (m, 1H), 3.67-3.78 (m, 1H), 3.59-3.67 (m, 1H), 3.40 (d, 3H), 3.10-3.29 (m, 3H), 2.52-2.68 (m, 2H), 2.10-2.42 (m, 4H), 1.91-2.08 (m, 3H). |
| 435a | 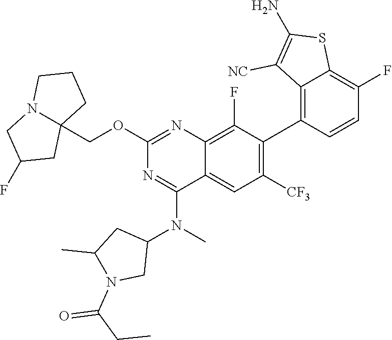 | |
| 436 | 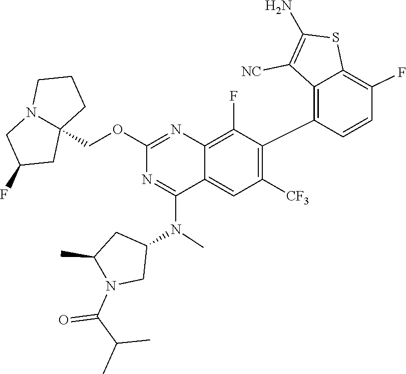 [SMILES: CC(C)C(=O)N1C[C@@H](N(C)c2nc(OC[C@@]34CCCN3C[C@H](F)C4)nc3c(F)c(-c4ccc(F)c5sc(N)c(C#N)c45)c(C(F)(F)F)cc23)C[C@@H]1C] | LCMS m/z [M + 1]: 746.2 1H NMR (400 MHz, CD3OD) δ 8.28 (d, 1H), 7.23 (m, 1H), 7.03 (m, 1H), 6.81-6.88 (m, 0.5H), 6.60- 6.68 (m, 0.5H), 6.33 (d, 1H), 5.98-6.06 (m, 1H), 5.50-5.83 (m, 1H), 5.40 (s, 0.5H), 5.27 (s, 0.5H), 4.50-4.60 (m, 1H), 3.84-3.92 (m, 1H), 3.67-3.78 (m, 1H), 3.59-3.67 (m, 1H), 3.40 (d, 3H), 3.10-3.29 (m, 3H), 2.52-2.68 (m, 2H), 2.10-2.42 (m, 4H), 1.91-2.08 (m, 3H). |
| 436a | 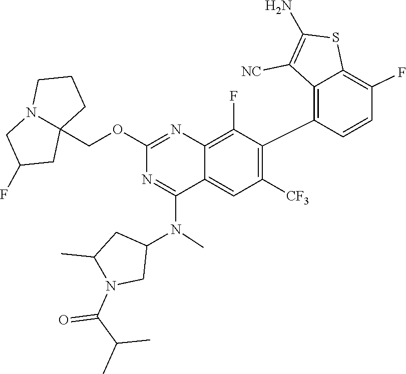 [SMILES: CC(C)C(=O)N1CC(N(C)c2nc(OCC34CCCN3CC(F)C4)nc3c(F)c(-c4ccc(F)c5sc(N)c(C#N)c45)c(C(F)(F)F)cc23)CC1C] | |
| 437 | 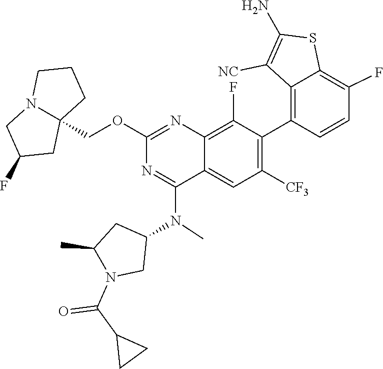 [SMILES: C[C@H]1C[C@H](N(C)c2nc(OC[C@@]34CCCN3C[C@H](F)C4)nc3c(F)c(-c4ccc(F)c5sc(N)c(C#N)c45)c(C(F)(F)F)cc23)CN1C(=O)C1CC1] | LCMS m/z [M + 1]: 744.1 1H NMR (400 MHz, CD3OD) δ 8.28 (d, 1H), 7.23 (m, 1H), 7.03 (m, 1H), 6.81-6.88 (m, 0.5H), 6.60- 6.68 (m, 0.5H), 6.33 (d, 1H), 5.98-6.06 (m, 1H), 5.50-5.83 (m, 1H), 5.40 (s, 0.5H), 5.27 (s, 0.5H), 4.50-4.60 (m, 1H), 3.84-3.92 (m, 1H), 3.67-3.78 (m, 1H), 3.59-3.67 (m, 1H), 3.40 (d, 3H), 3.10-3.29 (m, 3H), 2.52-2.68 (m, 2H), 2.10-2.42 (m, 4H), 1.91-2.08 (m, 3H). |
| 437a | 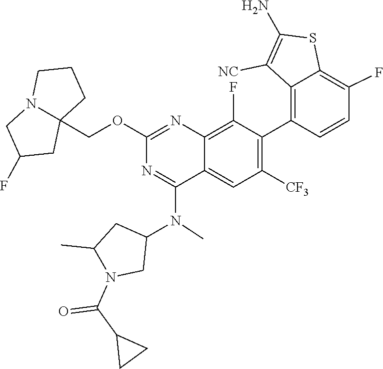 [SMILES: CC1CC(N(C)c2nc(OCC34CCCN3CC(F)C4)nc3c(F)c(-c4ccc(F)c5sc(N)c(C#N)c45)c(C(F)(F)F)cc23)CN1C(=O)C1CC1] | |
| 438 | 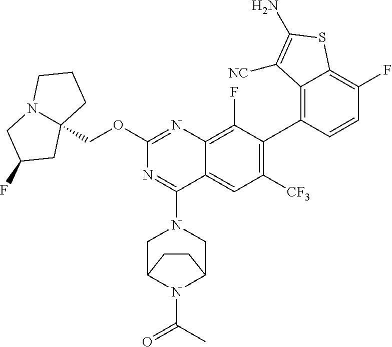 [SMILES: CC(=O)N1C2CCC1CN(c1nc(OC[C@@]34CCCN3C[C@H](F)C4)nc3c(F)c(-c4ccc(F)c5sc(N)c(C#N)c45)c(C(F)(F)F)cc13)C2] | LCMS m/z [M + 1]: 716.22 1H NMR (400 MHz, CD3OD) δ 7.70 (s, 1H), 7.24- 7.16 (m, 1H), 7.04-6.95 (m, 1H), 5.36 (s, 0.5H), 5.23 (s, 0.5H), 4.76 (s, 1H), 4.70-4.60 (m, 1H), 4.57-4.40 (m, 2H), 4.33-4.19 (m, 2H), 3.85-3.62 (m, 2H), 3.29- 3.13 (m, 3H), 3.05-2.95 (m, 1H), 2.35-2.10 (m, 6H), 2.07-1.81 (m, 7H). |
| 438a | 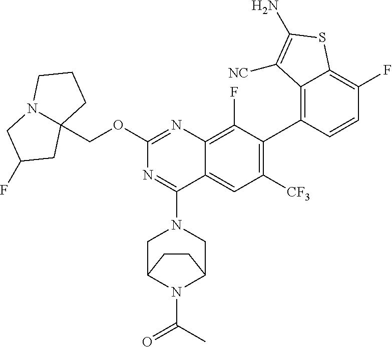 [SMILES: CC(=O)N1C2CCC1CN(c1nc(OCC34CCCN3CC(F)C4)nc3c(F)c(-c4ccc(F)c5sc(N)c(C#N)c45)c(C(F)(F)F)cc13)C2] | |
| 439 | 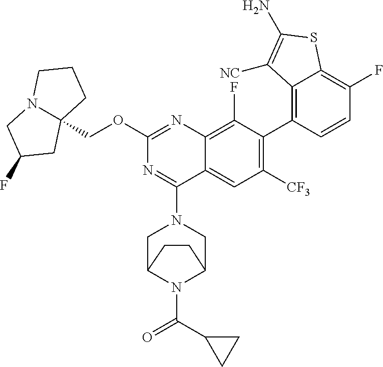 [SMILES: N#Cc1c(N)sc2c(F)ccc(-c3c(C(F)(F)F)cc4c(N5CC6CCC(C5)N6C(=O)C5CC5)nc(OC[C@@]56CCCN5C[C@H](F)C6)nc4c3F)c12] | LCMS m/z [M + 1]: 742.23 1H NMR (400 MHz, DMSO_d6) δ 8.21-7.96 (m, 3H), 7.30-7.21 (m, 1H), 7.18-7.08 (m, 1H), 5.35 (s, 0.5H), 5.21 (s, 0.5H), 4.80 (s, 1H), 4.63 (s, 1H), 4.52- 4.34 (m, 2H), 4.20-3.99 (m, 2H), 3.83-3.50 (m, 2H), 3.20-2.93 (m, 3H), 2.82 (s, 1H), 2.23-1.57 (m, 11H), 0.88-0.67 (m, 4H). |
| 439a | 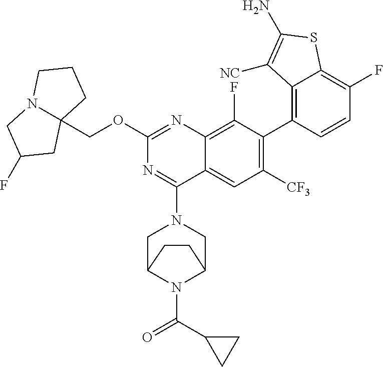 [SMILES: N#Cc1c(N)sc2c(F)ccc(-c3c(C(F)(F)F)cc4c(N5CC6CCC(C5)N6C(=O)C5CC5)nc(OCC56CCCN5CC(F)C6)nc4c3F)c12] | |
| 440 | 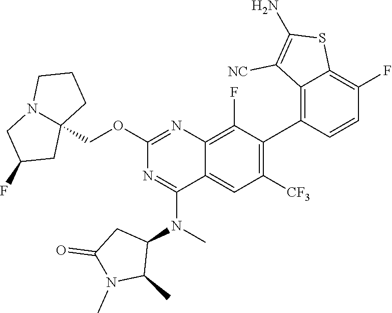 [SMILES: C[C@@H]1[C@H](N(C)c2nc(OC[C@@]34CCCN3C[C@H](F)C4)nc3c(F)c(-c4ccc(F)c5sc(N)c(C#N)c45)c(C(F)(F)F)cc23)CC(=O)N1C] | LCMS m/z [M + 1]: 704.1 1HNMR (400 MHz, CD3OD) δ 8.35 (s, 1H), 7.22- 7.19 (m, 1H), 7.01 (t, 1H), 5.49-5.43 (s, 1H), 5.41 (s, 0.5 H), 5.27 (s, 0.5H), 4.37-4.27 (m, 3H), 3.51 (s, 3H), 3.15-2.91 (m, 3H), 2.86 (s, 3H), 2.88-2.81 (m, 1H), 2.44-1.85 (m, 8H), 1.23 (t, 3H). |
| 440a | 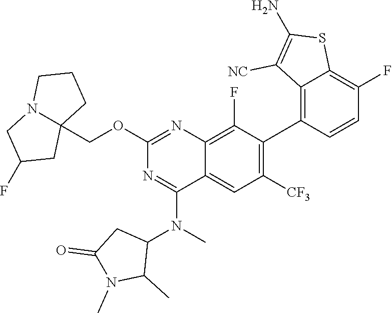 [SMILES: CC1C(N(C)c2nc(OCC34CCCN3CC(F)C4)nc3c(F)c(-c4ccc(F)c5sc(N)c(C#N)c45)c(C(F)(F)F)cc23)CC(=O)N1C] | |
| 441 | 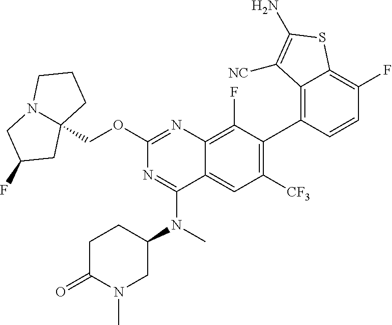 [SMILES: CN1C[C@H](N(C)c2nc(OC[C@@]34CCCN3C[C@H](F)C4)nc3c(F)c(-c4ccc(F)c5sc(N)c(C#N)c45)c(C(F)(F)F)cc23)CCC1=O] | LCMS m/z [M + 1]: 704.1 1H NMR (400 MHz, CD3OD) δ 8.35 (s, 1H), 7.27- 7.17 (m, 1H), 7.02 (m, 1H), 5.44 (s, 0.5H), 5.31 (s, 0.5H), 5.13 (s, 1H), 4.50-4.29 (m, 2H), 3.69 (m, 2H), 3.53-3.45 (m, 4H), 3.43 (s, 2H), 3.13 (s, 1H), 3.00 (s, 3H), 2.65-2.53 (m, 2H), 2.49-2.28 (m, 3H), 2.20 (m, 2H), 2.09 (s, 2H), 1.95 (s, 1H). |
| 441a | 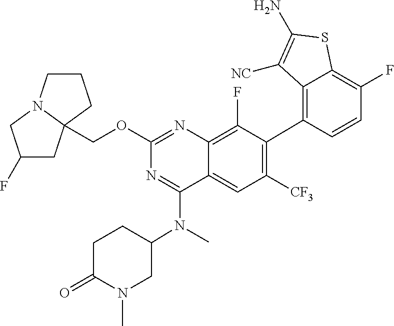 [SMILES: CN1CC(N(C)c2nc(OCC34CCCN3CC(F)C4)nc3c(F)c(-c4ccc(F)c5sc(N)c(C#N)c45)c(C(F)(F)F)cc23)CCC1=O] | |
| 442 | 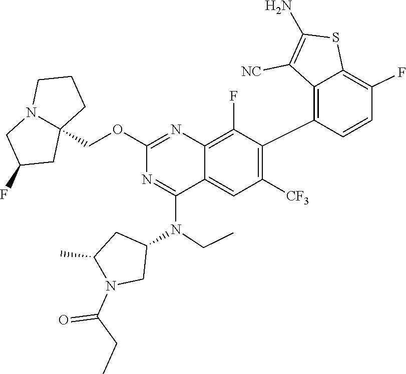 [SMILES: CCC(=O)N1C[C@@H](N(CC)c2nc(OC[C@@]34CCCN3C[C@H](F)C4)nc3c(F)c(-c4ccc(F)c5sc(N)c(C#N)c45)c(C(F)(F)F)cc23)C[C@H]1C] | LCMS m/z [M + 1]: 746.1 1H NMR (400 MHz, CD3OD) δ 8.23 (s, 1H), 7.19- 7.23 (m, 1H), 7.01 (t, 1H), 5.37 (s, 0.5H), 5.24 (s, 0.5H), 4.09-4.36 (m, 5H), 3.85-3.88 (m, 2H), 3.67- 3.73 (m, 1H), 3.36-3.47 (m, 1H), 3.23 (s, 2H), 3.01- 3.06 (m, 1H), 2.57-2.67 (m, 1H), 2.22-2.48 (m, 4H), 2.01-2.16 (m, 4H), 1.86-1.92 (m, 1H), 1.40-1.46 (m, 6H), 1.28 (s, 3H). |
| 442a | 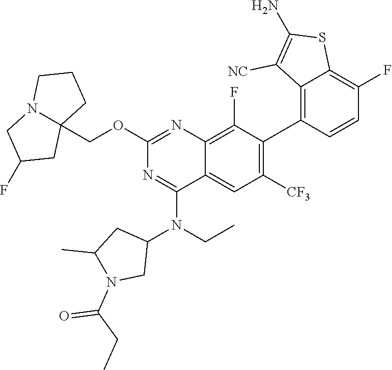 [SMILES: CCC(=O)N1CC(N(CC)c2nc(OCC34CCCN3CC(F)C4)nc3c(F)c(-c4ccc(F)c5sc(N)c(C#N)c45)c(C(F)(F)F)cc23)CC1C] | |
| 443 | 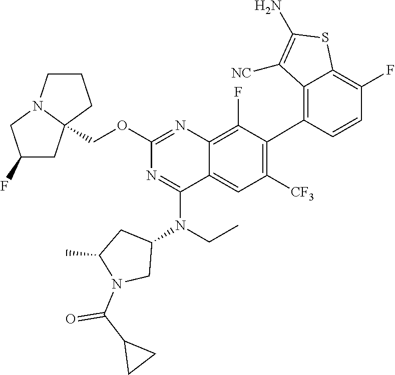 | LCMS m/z [M + 1]: 744.0 1H NMR (400 MHz, CD3OD) δ 8.30 (s, 1H), 7.23- 7.20 (m, 1H), 7.03-6.98 (m, 1H), 5.36 (s, 0.5H), 5.23 (s, 0.5H), 5.17-5.04 (m, 1H), 4.50-4.08 (m, 4H), 3.86-3.67 (m, 1H), 3.52-3.38 (m, 3H), 3.29-3.15 (m, 3H), 3.07-3.97 (m, 1H), 2.69-2.51 (m, 1H), 2.41-1.79 (m, 8H), 1.59-1.35 (m, 3H), 1.09-0.78 (m, 4H). |
| 444 | 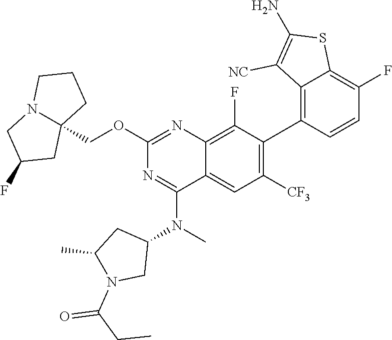 | LCMS m/z [M + 1]: 732.1 1H NMR (400 MHz, CD3OD) δ 8.30 (s, 1H), 7.25- 7.18 (m, 1H), 7.05-6.97 (m, 1H), 5.40 (s, 0.5H), 5.27 (s, 0.5H), 5.13-5.06 (m, 1H), 4.40-4.35 (m, 1H), 4.31-4.23 (m, 1H), 4.19-4.04 (m, 2H), 3.67-3.59 (m, 1H), 3.50-3.37 (m, 4H), 3.14-3.06 (m, 1H), 2.69-2.58 (m, 1H), 2.49-2.13 (m, 6H), 2.11-1.81 (m, 5H), 1.49- 1.36 (m, 3H), 1.20-1.08 (m, 3H). |
| 445 | 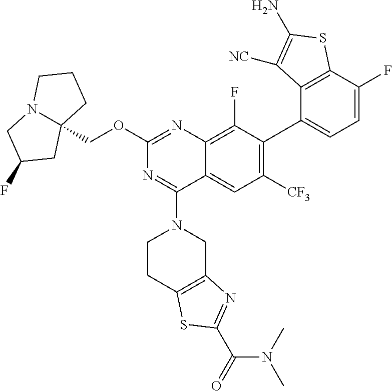 | LCMS m/z [M + 1]: 773.2 1H NMR (400 MHz, CD3OD) δ 8.30 (s, 1H), 7.22- 7.26 (m, 1H), 7.03 (t, J = 8.48 Hz, 1H), 5.57 (d, J = 52.2 Hz, 1H), 5.22 (s, 2H), 4.64-4.79 (m, 2H), 4.29- 4.37 (m, 2H), 3.83-4.09 (m, 3H), 3.57 (s, 3H), 3.43- 3.51 (m, 1H), 3.32-3.34 (m, 2H), 3.13 (s, 3H), 2.54- 2.81 (m, 2H), 2.17-2.49 (m, 4H). |
| 445a | 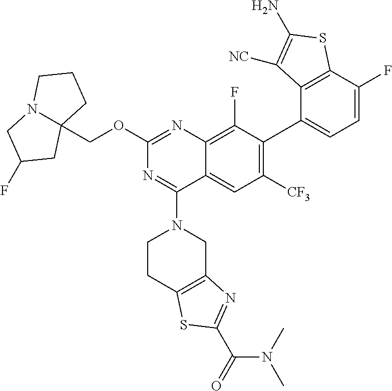 | |
| 446 | 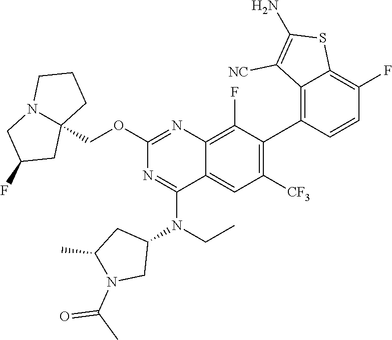 | LCMS m/z [M + 1]: 732.1 1H NMR (400 MHz, CD3OD) δ 8.23 (s, 1H), 7.20- 7.23 (m, 1H), 7.01 (t, 1H), 5.38 (s, 0.5H), 5.25 (s, 0.5H), 4.02-4.36 (m, 4H), 3.63-3.92 (m, 3H), 3.35- 3.47 (m, 1H), 3.05-3.10 (m, 1H), 2.35-2.76 (m, 2H), 2.03-2.30 (m, 10H), 1.90 (s, 1H), 1.58 (s, 1H), 1.40- 1.48 (m, 6H). |
| 446a | 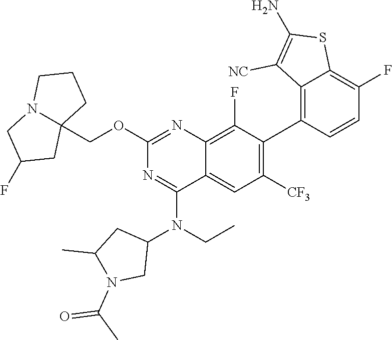 | |
| 447 | 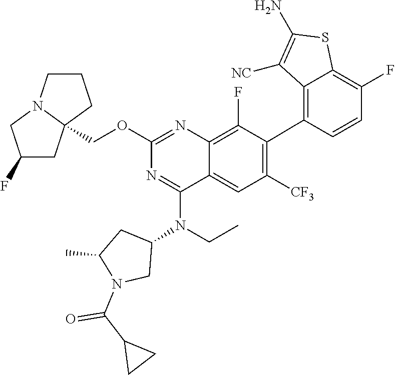 | LCMS m/z [M + 1]: 746.1 1H NMR (400 MHz, CD3OD) δ 8.30 (s, 1H), 7.24- 7.18 (m, 1H), 7.04-6.98 (m, 1H), 5.40 (s, 0.5H), 5.27 (s, 0.5H), 5.13-4.95 (m, 2H), 4.45-4.06 (m, 5H), 3.68-3.59 (m, 1H), 3.50-3.36 (m, 4H), 3.13-3.08 (m, 1H), 2.77-1.85 (m, 13H), 1.22-1.07 (m, 3H). |
| 447a | 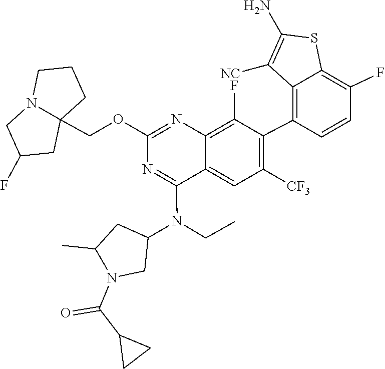 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)C2CC2)C1] | |
| 448 | 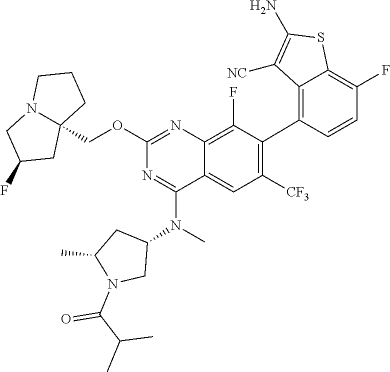 [SMILES: CC(C)C(=O)N1C[C@@H](N(C)c2nc(OC[C@@]34CCCN3C[C@H](F)C4)nc3c(F)c(-c4ccc(F)c5sc(N)c(C#N)c45)c(C(F)(F)F)cc23)C[C@H]1C] | LCMS m/z [M + 1]: 746.1 1H NMR (400 MHz, CD3OD) δ 8.30 (s, 1H), 7.24- 7.18 (m, 1H), 7.04-6.98 (m, 1H), 5.40 (s, 0.5H), 5.27 (s, 0.5H), 5.13-4.95 (m, 2H), 4.45-4.06 (m, 5H), 3.68-3.59 (m, 1H), 3.50-3.36 (m, 4H), 3.13-3.08 (m, 1H), 2.77-1.85 (m, 13H), 1.22-1.07 (m, 3H). |
| 449 | 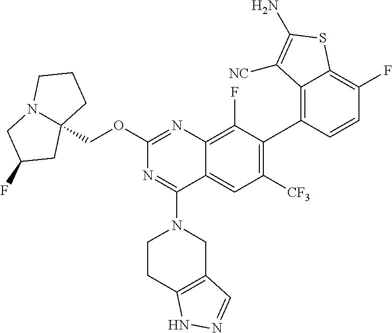 | LCMS m/z [M + 1]: 685.0 1H NMR (400 MHz, CD3OD) δ 8.27 (s, 1H), 7.23- 7.26 (m, 1H), 7.04 (t, 1H), 5.63 (s, 0.5H), 5.50 (s, 0.5H), 4.85 (d, 1H), 4.73 (t, 1H), 4.60-4.68 (m, 1H), 3.99-4.10 (m, 1H), 3.85-3.94 (m, 2H), 3.42-3.51 (m, 3H), 3.14 (s, 1H), 2.30-2.64 (m, 8H), 2.15 (s, 2H), 1.80-1.90 (m, 2H). |
| 449a | 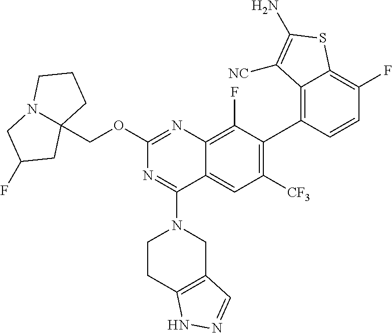 | |
| 450 | 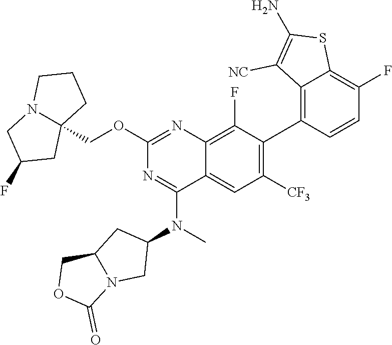 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@@H]1C[C@@H]2COC(=O)N2C1] | LCMS m/z [M + 1]: 718.1 1H NMR (400 MHz, CD3OD) δ 8.30 (s, 1H), 7.22 (m, 1H), 7.02 (m, 1H), 5.53 (s, 0.5H), 5.40 (s, 0.5H), 5.29 (m, 1H), 4.65-4.58 (m, 1H), 4.56-4.43 (m, 2H), 4.43-4.38 (m, 1H), 4.20 (s, 1H), 3.69 (m, 6H), 3.46 (s, 3H), 2.69-2.14 (m, 7H), 2.05 (s, 1H). |
| 450a | 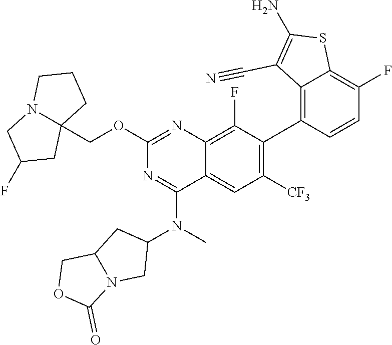 [SMILES: CN(c1nc(OCC23CCCN2CC(F)C3)nc2c(F)c(-c3ccc(F)c4sc(N)c(C#N)c34)c(C(F)(F)F)cc12)C1CC2COC(=O)N2C1] | |
| 451 | 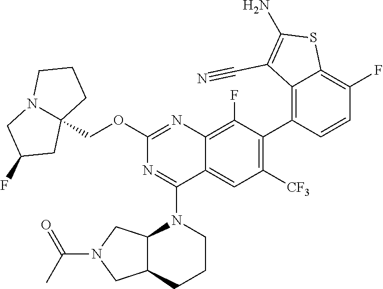 [SMILES: CC(=O)N1C[C@@H]2CCCN(c3nc(OC[C@@]45CCCN4C[C@H](F)C5)nc4c(F)c(-c5ccc(F)c6sc(N)c(C#N)c56)c(C(F)(F)F)cc34)[C@@H]2C1] | LCMS m/z [M + 1]: 730.1 1H NMR (400 MHz, CD3OD) δ 8.12-8.09 (m, 1H), 7.23-7.20 (m, 1H), 7.03-6.99 (m, 1H), 5.37 (s, 0.48H), 5.23 (s, 0.63H), 5.11-5.08 (m, 1H), 4.33- 4.23 (m, 3H), 3.98-3.89 (m, 2H), 3.76-3.71 (m, 2H), 3.55-3.47 (m, 3H), 3.25-3.13 (m, 3H), 3.02-2.99 (m, 1H), 2.52 (s, 1H), 2.35-1.87 (m, 1H), 1.64 (s, 1H). |
| 451a | 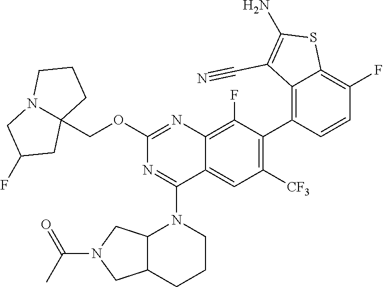 [SMILES: CC(=O)N1CC2CCCN(c3nc(OCC45CCCN4CC(F)C5)nc4c(F)c(-c5ccc(F)c6sc(N)c(C#N)c56)c(C(F)(F)F)cc34)C2C1] | |
| 452 | 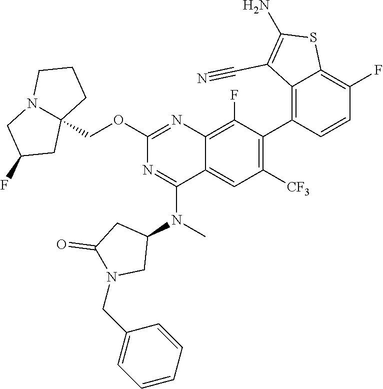 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@@H]1CC(=O)N(Cc2ccccc2)C1] | LCMS m/z [M + 1]: 766.1 1H NMR (400 MHz, CD3OD) δ 8.25 (s, 1H), 7.45- 7.25 (m, 5H), 7.23-7.18 (m, 1H), 7.02 (t, 1H), 5.49 (s, 0.5H), 5.36 (s, 0.5H), 5.33-5.26 (m, 1H), 4.67- 4.61 (s, 1H), 4.52-4.36 (m, 3H), 3.89-3.83 (m, 1H), 3.62-3.52 (m, 3H), 3.40 (s, 3H), 3.06-2.98 (m, 1H), 2.92-2.85 (m, 1H), 2.56-1.95 (m, 8H). |
| 452a | 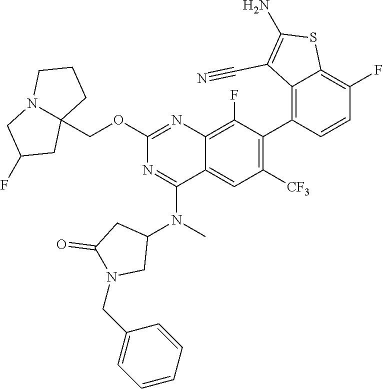 [SMILES: CN(c1nc(OCC23CCCN2CC(F)C3)nc2c(F)c(-c3ccc(F)c4sc(N)c(C#N)c34)c(C(F)(F)F)cc12)C1CC(=O)N(Cc2ccccc2)C1] | |
| 453 | 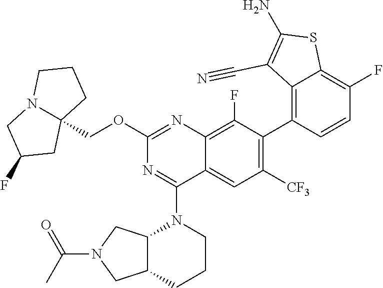 [SMILES: CC(=O)N1C[C@H]2CCCN(c3nc(OC[C@@]45CCCN4C[C@H](F)C5)nc4c(F)c(-c5ccc(F)c6sc(N)c(C#N)c56)c(C(F)(F)F)cc34)[C@H]2C1] | LCMS m/z [M + 1]: 730.23 1H NMR (400 MHz, CD3OD) δ 8.14-8.05 (m, 1H), 7.25-7.17 (m, 1H), 7.05-6.96 (m, 1H), 5.36 (s, 0.5H), 5.26-5.03 (m, 1.5H), 4.36-4.15 (m, 3H), 4.02-3.45 (m, 5H), 3.28-3.14 (m, 3H), 3.06-2.94 (m, 1H), 2.61- 2.44 (m, 1H), 2.37-2.04 (m, 6H), 2.03-1.79 (m, 6H), 1.71-1.57 (m, 1H). |
| 454 | 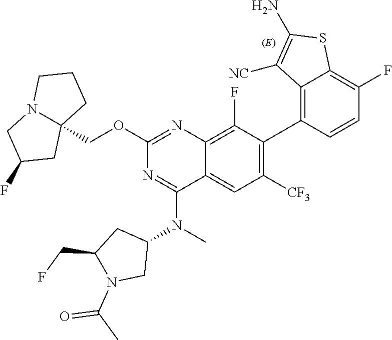 [SMILES: CC(=O)N1C[C@@H](N(C)c2nc(OC[C@@]34CCCN3C[C@H](F)C4)nc3c(F)c(-c4ccc(F)c5sc(N)c(C#N)c45)c(C(F)(F)F)cc23)C[C@@H]1CF] | LCMS m/z [M + 1]: 736.22 1H NMR (400 MHz, CD3OD) δ 8.33-8.26 (m, 1H), 7.25-7.17 (m, 1H), 7.04-6.96 (m, 1H), 5.63-5.46 (m, 1H), 5.37 (s, 0.5H), 5.23 (s, 0.5H), 4.79-4.40 (m, 3H), 4.36-4.21 (m, 2H), 4.07-3.70 (m, 2H), 3.50-3.37 (m, 3H), 3.28-3.12 (m, 3H), 3.06-2.95 (m, 1H), 2.60- 2.07 (m, 8H), 2.04-1.85 (m, 3H). |
| 454a | 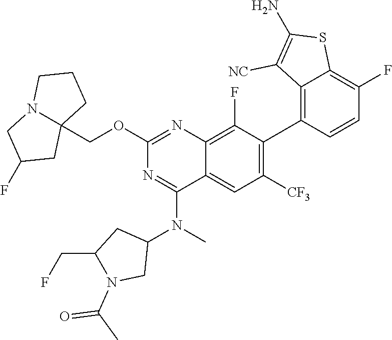 [SMILES: CC(=O)N1CC(N(C)c2nc(OCC34CCCN3CC(F)C4)nc3c(F)c(-c4ccc(F)c5sc(N)c(C#N)c45)c(C(F)(F)F)cc23)CC1CF] | |
| 455 | 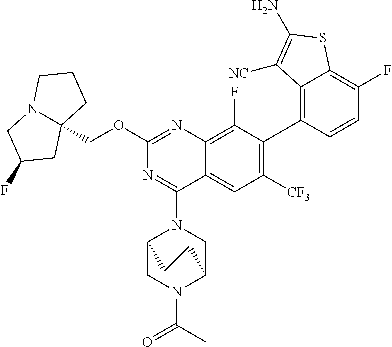 [SMILES: CC(=O)N1C[C@@H]2CC[C@H]1CN2c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 716.1 1H NMR (400 MHz, CD3OD) δ 8.34 (s, 1H), 7.18- 7.21 (m, 1H), 7.01 (t, 1H), 5.45 (s, 0.5H), 5.32 (s, 0.5H), 5.26 (s, 1H), 4.03-4.47 (m, 5H), 3.85 (s, 1H), 3.47-3.71 (m, 4H), 3.12-3.16 (m, 1H), 2.35-2.46 (m, 3H), 2.20-2.26 (m, 1H), 2.11-2.16 (m, 4H), 1.96-2.07 (m, 5H). |
| 455a | 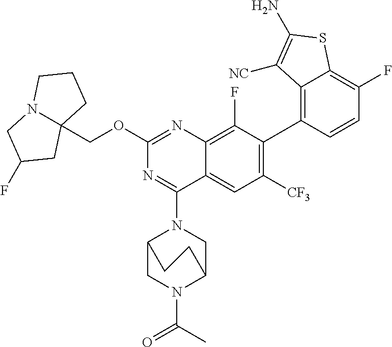 [SMILES: CC(=O)N1CC2CCC1CN2c1nc(OCC23CCCN2CC(F)C3)nc2c(F)c(-c3ccc(F)c4sc(N)c(C#N)c34)c(C(F)(F)F)cc12] | |
| 456 | 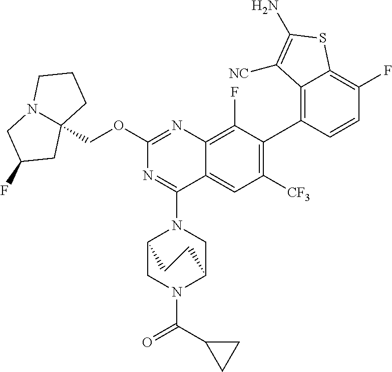 [SMILES: N#Cc1c(N)sc2c(F)ccc(-c3c(C(F)(F)F)cc4c(N5C[C@@H]6CC[C@H]5CN6C(=O)C5CC5)nc(OC[C@@]56CCCN5C[C@H](F)C6)nc4c3F)c12] | LCMS m/z [M + 1]: 742.1 1H NMR (400 MHz, CD3OD) δ 8.34 (s, 1H), 7.18- 7.23 (m, 1H), 7.01 (t, 1H), 5.44 (s, 0.5H), 5.30 (s, 1H), 5.25 (s, 0.5H), 4.75-4.81 (m, 1H), 4.40-4.54 (m, 2H), 4.04-4.35 (m, 3H), 3.71-3.87 (m, 1H), 3.42-3.55 (m, 3H), 3.12 (s, 1H), 2.31-2.44 (m, 3H), 2.18-2.22 (m, 1H), 2.04-2.07 (m, 4H), 1.83-2.00 (m, 3H), 0.83- 0.93 (m, 4H). |
| 456a | 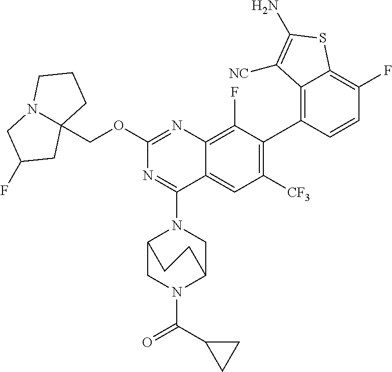 [SMILES: N#Cc1c(N)sc2c(F)ccc(-c3c(C(F)(F)F)cc4c(N5CC6CCC5CN6C(=O)C5CC5)nc(OCC56CCCN5CC(F)C6)nc4c3F)c12] | |
| 457 | 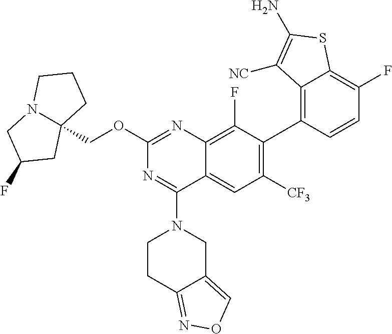 [SMILES: N#Cc1c(N)sc2c(F)ccc(-c3c(C(F)(F)F)cc4c(N5CCc6nocc6C5)nc(OC[C@@]56CCCN5C[C@H](F)C6)nc4c3F)c12] | LCMS m/z [M + 1]: 686.0 1H NMR (400 MHz, CD3OD) δ 8.54 (s, 1H), 8.20 (s, 1H), 7.25-7.19 (m, 1H), 7.05-6.98 (m, 1H), 5.38 (s, 0.5H), 5.25 (s, 0.5H), 5.07 (s, 2H), 4.40-4.33 (m, 1H), 4.33-4.21 (m, 2H), 4.20-4.11 (m, 1H), 3.30-3.19 (m, 5H), 3.07-3.00 (m, 1H), 2.40-2.21 (m, 2H), 2.19- 2.13 (m, 1H), 2.05-1.88 (m, 3H). |
| 457a | 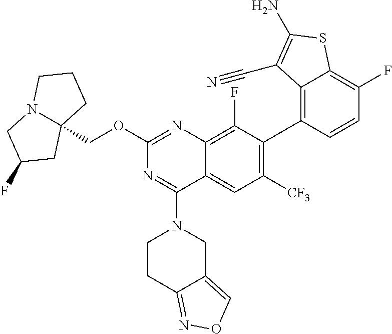 [SMILES: N#Cc1c(N)sc2c(F)ccc(-c3c(C(F)(F)F)cc4c(N5CCc6nocc6C5)nc(OC[C@@]56CCCN5C[C@H](F)C6)nc4c3F)c12] | |
| 458 | 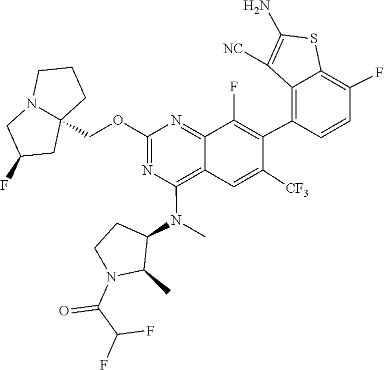 [SMILES: C[C@@H]1[C@H](N(C)c2nc(OC[C@@]34CCCN3C[C@H](F)C4)nc3c(F)c(-c4ccc(F)c5sc(N)c(C#N)c45)c(C(F)(F)F)cc23)CCN1C(=O)C(F)F] | LCMS m/z [M + 1]: 754.2 1H NMR (400 MHz, CD3OD) δ 8.41 (s, 1H), 7.21- 7.25 (m, 1H), 7.03 (t, J = 9.12 Hz, 1H), 6.23-6.55 (m, 1H), 5.57 (d, J = 55.48 Hz, 1H), 5.03-5.16 (m, 1H), 4.60-4.83 (m, 3H), 3.44-4.04 (m, 9H), 2.11-2.76 (m, 8H), 1.10-1.21 (m, 3H). |
| 458a | 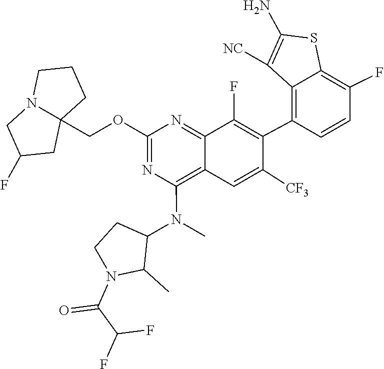 [SMILES: CC1C(N(C)c2nc(OCC34CCCN3CC(F)C4)nc3c(F)c(-c4ccc(F)c5sc(N)c(C#N)c45)c(C(F)(F)F)cc23)CCN1C(=O)C(F)F] | |
| 459 | 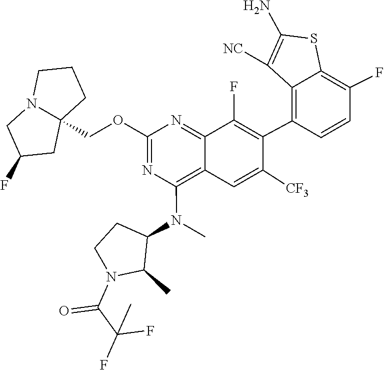 [SMILES: C[C@@H]1[C@H](N(C)c2nc(OC[C@@]34CCCN3C[C@H](F)C4)nc3c(F)c(-c4ccc(F)c5sc(N)c(C#N)c45)c(C(F)(F)F)cc23)CCN1C(=O)C(C)(F)F] | LCMS m/z [M + 1]: 768.2 1H NMR (400 MHz, CD3OD) δ 8.41 (s, 1H), 7.21- 7.25 (m, 1H), 7.03 (t, J = 9.12 Hz, 1H), 5.57 (d, J = 55.48 Hz, 1H), 5.03-5.33 (m, 1H), 4.56-4.81 (m, 3H), 3.43-4.13 (m, 9H), 2.11-2.74 (m, 8H), 1.78-1.93 (m, 3H), 1.09-1.19 (m, 3H). |
| 459a | 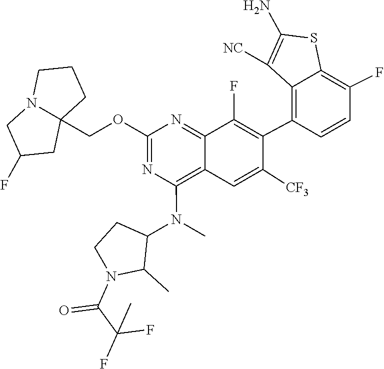 [SMILES: CC1C(N(C)c2nc(OCC34CCCN3CC(F)C4)nc3c(F)c(-c4ccc(F)c5sc(N)c(C#N)c45)c(C(F)(F)F)cc23)CCN1C(=O)C(C)(F)F] | |
| 460 | 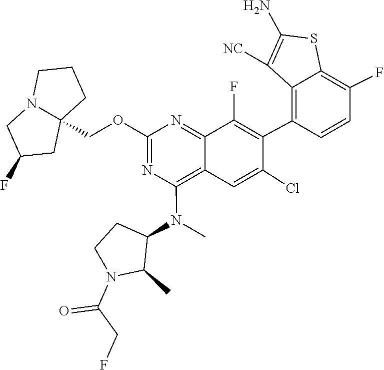 [SMILES: C[C@@H]1[C@H](N(C)c2nc(OC[C@@]34CCCN3C[C@H](F)C4)nc3c(F)c(-c4ccc(F)c5sc(N)c(C#N)c45)c(Cl)cc23)CCN1C(=O)CF] | LCMS m/z [M + 1]: 702.2 1HNMR (400 MHz, CD3OD) δ 8.17 (s, 1H), 7.21- 7.25 (m, 1H), 7.06 (t, J = 8.88 Hz, 1H), 5.56 (d, J = 52.32 Hz, 1H), 5.00-5.11 (m, 3H), 4.58-4.75 (m, 3H), 3.46-4.04 (m, 9H), 2.08-2.72 (m, 8H), 1.06-1.11 (m, 3H). |
| 460a | 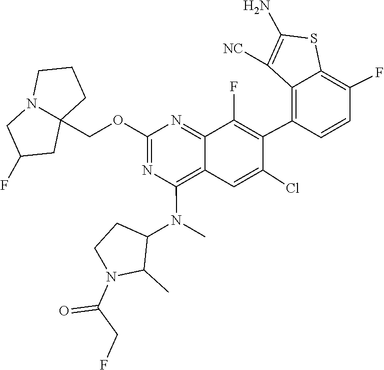 [SMILES: CC1C(N(C)c2nc(OCC34CCCN3CC(F)C4)nc3c(F)c(-c4ccc(F)c5sc(N)c(C#N)c45)c(Cl)cc23)CCN1C(=O)CF] | |
| 461 | 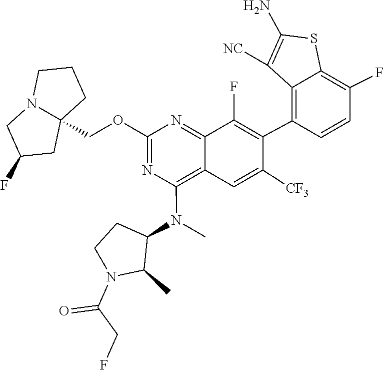 [SMILES: C[C@@H]1[C@H](N(C)c2nc(OC[C@@]34CCCN3C[C@H](F)C4)nc3c(F)c(-c4ccc(F)c5sc(N)c(C#N)c45)c(C(F)(F)F)cc23)CCN1C(=O)CF] | LCMS m/z [M + 1]: 736.3 1H NMR (400 MHz, CD3OD) δ 8.34 (s, 1H), 7.20- 7.23 (m, 1H), 7.01 (t, J = 8.88 Hz, 1H), 5.34 (d, J = 53.2 Hz, 1H), 5.05 (d, J = 46.68 Hz, 2H), 5.01-5.13 (m, 1H), 4.59-4.82 (m, 2H), 4.25-4.41 (m, 2H), 3.24-3.75 (m, 7H), 3.03-3.06 (m, 3H), 1.91-2.48 (m, 8H), 1.07- 1.12 (m, 3H). |
| 461a | 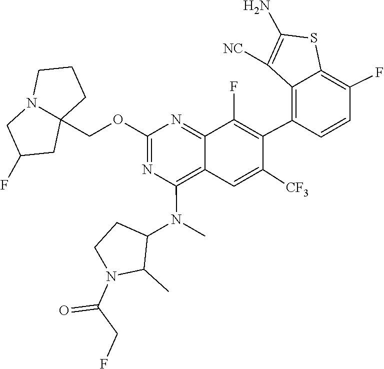 [SMILES: CC1C(N(C)c2nc(OCC34CCCN3CC(F)C4)nc3c(F)c(-c4ccc(F)c5sc(N)c(C#N)c45)c(C(F)(F)F)cc23)CCN1C(=O)CF] | |
| 462 | 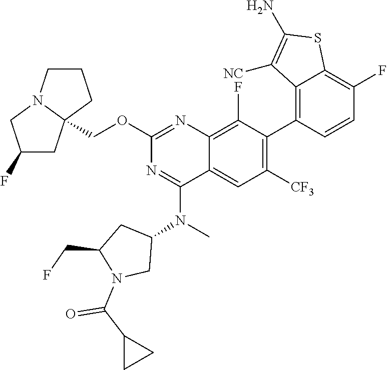 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@H]1C[C@H](CF)N(C(=O)C2CC2)C1] | LCMS m/z [M + 1]: 762.24 1H NMR (400 MHz, CD3OD) δ 8.35-8.28 (m, 1H), 7.24-7.19 (m, 1H), 7.04-6.97 (m, 1H), 5.65-5.48 (m, 1H), 5.39 (s, 0.5H), 5.25 (s, 0.5H), 4.76-4.41 (m, 3H), 4.38-4.26 (m, 2H), 4.25-4.17 (m, 1H), 3.99-3.85 (m, 1H), 3.47 (m, 2H), 3.43-3.38 (m, 1H), 3.28-3.19 (m, 2H), 3.12-2.99 (m, 1H), 2.68-1.84 (m, 10H), 0.96-0.84 (m, 4H). |
| 462a | 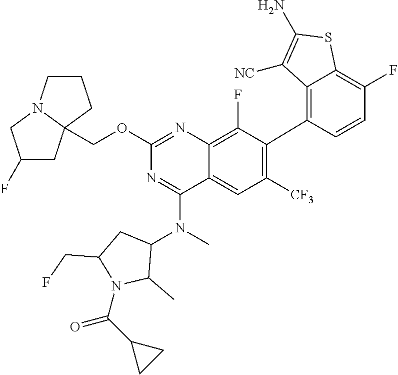 [SMILES: CC1C(N(C)c2nc(OCC34CCCN3CC(F)C4)nc3c(F)c(-c4ccc(F)c5sc(N)c(C#N)c45)c(C(F)(F)F)cc23)CC(CF)N1C(=O)C1CC1] | |
| 463 | 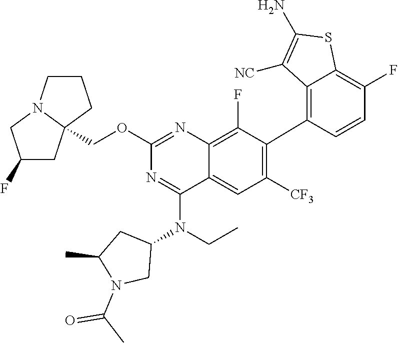 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@H]1C[C@H](C)N(C(C)=O)C1] | LCMS m/z [M + 1]: 732.25 1H NMR (400 MHz, CD3OD) δ 8.27-8.21 (m, 1H), 7.26-7.20 (m, 1H), 7.06-6.99 (m, 1H), 5.40 (s, 0.5H), 5.29-5.18 (m, 1.5H), 4.46-4.25 (m, 3H), 4.10-3.56 (m, 5H), 3.29-3.18 (m, 2H), 3.11-3.03 (m, 1H), 2.78- 2.61 (m, 1H), 2.43-2.23 (m, 2H), 2.19-1.98 (m, 7H), 1.97-1.85 (m, 1H), 1.51-1.40 (m, 3H), 1.39-1.30 (m, 3H). |
| 464 | 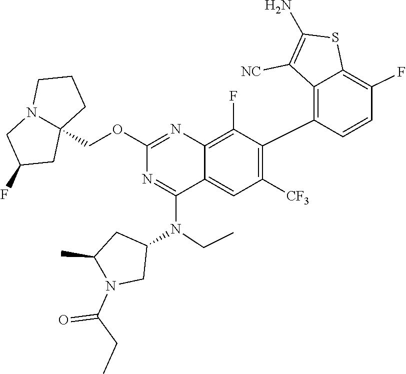 [SMILES: CCC(=O)N1C[C@@H](N(CC)c2nc(OC[C@@]34CCCN3C[C@H](F)C4)nc3c(F)c(-c4ccc(F)c5sc(N)c(C#N)c45)c(C(F)(F)F)cc23)C[C@@H]1C] | LCMS m/z [M + 1]: 746.26 1H NMR (400 MHz, CD3OD) δ 8.31-8.17 (m, 1H), 7.26-7.18 (m, 1H), 7.05-6.97 (m, 1H), 5.41-5.36 (m, 0.5H), 5.31-5.15 (m, 1.5H), 4.49-4.17 (m, 3H), 4.07- 3.64 (m, 4H), 3.30-3.15 (m, 3H), 3.09-3.00 (m, 1H), 2.73-2.57 (m, 1H), 2.49-1.87 (m, 9H), 1.48-1.38 (m, 3H), 1.37-1.29 (m, 3H), 1.19-1.09 (m, 3H). |
| 465 | 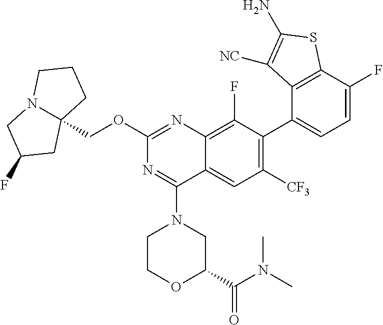 [SMILES: CN(C)C(=O)[C@H]1CN(c2nc(OC[C@@]34CCCN3C[C@H](F)C4)nc3c(F)c(-c4ccc(F)c5sc(N)c(C#N)c45)c(C(F)(F)F)cc23)CCO1] | LCMS m/z [M + 1]: 720.1 1H NMR (400 MHz, CD3OD) δ 8.22 (s, 1H), 7.26- 7.16 (m, 1H), 7.00 (t, J = 9.0 Hz, 1H), 5.37 (m, 1H), 5.24 (m, 1H), 4.68-4.57 (m, 1H), 4.47-4.44 (m, 1H), 4.40-4.14 (m, 3H), 4.09-3.98 (m, 1H), 3.94-3.67 (m, 3H), 3.49-3.31 (m, 2H), 3.28-3.18 (m, 2H), 3.23-3.10 (m, 3H), 3.06-2.92 (s, 3H), 2.38-2.10 (m, 3H), 2.08- 1.81 (s, 3H). |
| 465a | 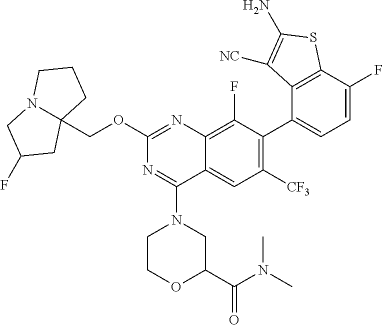 [SMILES: CN(C)C(=O)C1CN(c2nc(OCC34CCCN3CC(F)C4)nc3c(F)c(-c4ccc(F)c5sc(N)c(C#N)c45)c(C(F)(F)F)cc23)CCO1] | |
| 466 | 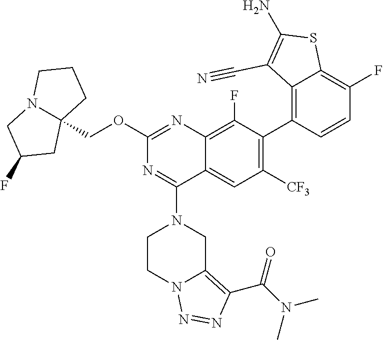 [SMILES: CN(C)C(=O)c1nnn2c1CN(c1nc(OC[C@@]34CCCN3C[C@H](F)C4)nc3c(F)c(-c4ccc(F)c5sc(N)c(C#N)c45)c(C(F)(F)F)cc13)CC2] | LCMS m/z [M + 1]: 757.4 1H NMR (400 MHz, CD3OD) δ 8.26 (s, 1H), 7.26- 7.16 (m, 1H), 7.01 (t, J = 8.8 Hz, 1H), 5.42-5.23 (m, 3H), 4.83-4.68 (m, 2H), 4.43 (s, 2H), 4.37-4.21 (m, 2H), 3.61-3.45 (m, 3H), 3.28-3.16 (m, 3H), 3.10 (s, 3H), 3.05-2.96 (m, 1H), 2.37-2.09 (m, 3H), 2.04-1.84 (s, 3H). |
| 466a | 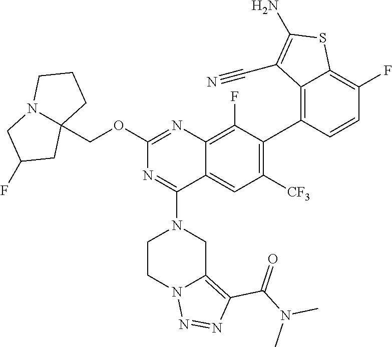 [SMILES: CN(C)C(=O)c1nnn2c1CN(c1nc(OCC34CCCN3CC(F)C4)nc3c(F)c(-c4ccc(F)c5sc(N)c(C#N)c45)c(C(F)(F)F)cc13)CC2] | |
| 467 | 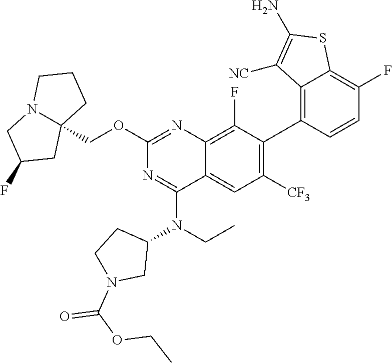 [SMILES: CCOC(=O)N1CC[C@H](N(CC)c2nc(OC[C@@]34CCCN3C[C@H](F)C4)nc3c(F)c(-c4ccc(F)c5sc(N)c(C#N)c45)c(C(F)(F)F)cc23)C1] | LCMS m/z [M + 1]: 748.1 1H NMR (400 MHz, CD3OD) δ 8.24 (s, 1H), 7.22 (s, 1H), 7.02 (m, 1H), 5.43 (s, 0.5H), 5.30 (s, 0.5H), 5.03 (m, 1H), 4.36 (s, 2H), 4.15 (m, 2H), 3.95-3.71 (m, 4H), 3.57 (s, 1H), 3.44 (m, 3H), 3.12 (m, 2H), 2.36 (m, 4H), 2.19 (m, 1H), 2.07 (m, 2H), 1.95 (m, 1H), 1.45 (s, 3H), 1.29 (s, 3H). |
| 467a | 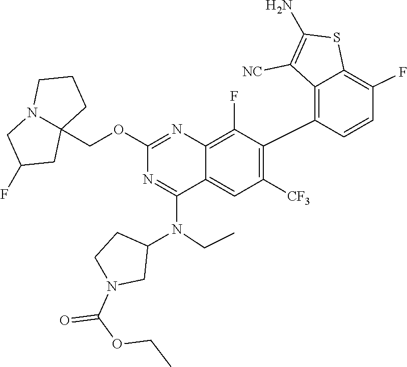 [SMILES: CCOC(=O)N1CCC(N(CC)c2nc(OCC34CCCN3CC(F)C4)nc3c(F)c(-c4ccc(F)c5sc(N)c(C#N)c45)c(C(F)(F)F)cc23)C1] | |
| 468 | 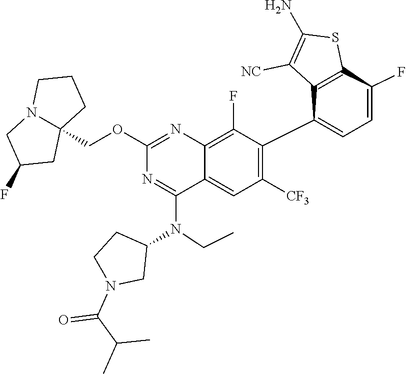 | LCMS m/z [M + 1]: 746.1 1H NMR (400 MHz, CD3OD) δ 8.25 (s, 1H), 7.26- 7.18 (m, 1H), 7.01 (m, 1H), 5.40 (s, 0.5H), 5.27 (s, 0.5H), 5.07 (m, 1H), 4.40-4.22 (m, 2H), 4.15-3.52 (m, 6H), 3.47 (s, 3H), 3.08 (s, 1H), 2.88-2.73 (m, 1H), 2.53-2.12 (m, 5H), 1.99 (m, 3H), 1.51-1.41 (m, 3H), 1.14 (m, 6H). |
| 469 | 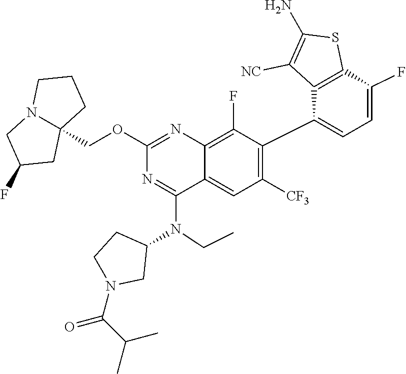 | LCMS m/z [M + 1]: 746.2 1H NMR (400 MHz, CD3OD) δ 8.25 (m, 1H), 7.25- 7.17 (m, 1H), 7.01 (m, 1H), 5.38 (s, 0.5H), 5.25 (s, 0.5H), 5.11-4.98 (m, 1H), 4.38-4.14 (m, 2H), 4.04- 3.81 (m, 3H), 3.76-3.35 (m, 3H), 3.28-3.19 (m, 3H), 3.04 (s, 1H), 2.90-2.73 (m, 1H), 2.60-2.09 (m, 5H), 1.96 (m, 3H), 1.45 (m, 3H), 1.18-1.09 (m, 6H). |
| 470 | 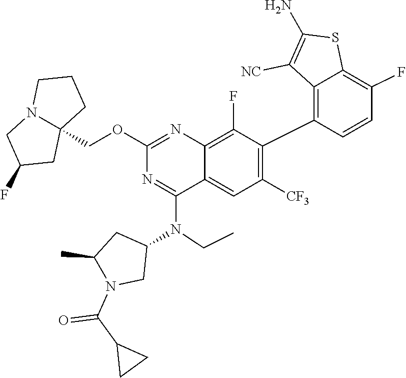 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@H]1C[C@H](C)N(C(=O)C2CC2)C1] | LCMS m/z [M + 1]: 758.26 1H NMR (400 MHz, CD3OD) δ 8.32-8.16 (m, 1H), 7.26-7.16 (m, 1H), 7.06-6.93 (m, 1H), 5.39 (s, 0.5H), 5.34-5.18 (s, 1.5H), 4.66-4.53 (m, 0.5H), 4.45-4.36 (m, 0.5H), 4.34-4.13 (m, 2H), 3.99-3.65 (m, 3H), 3.51-3.31 (m, 1H), 3.28-3.22 (m, 3H), 3.12-2.95 (m, 1H), 2.83-2.64 (m, 1H), 2.46-1.64 (m, 8H), 1.50-1.40 (m, 3H), 1.35-1.25. |
| 470a | 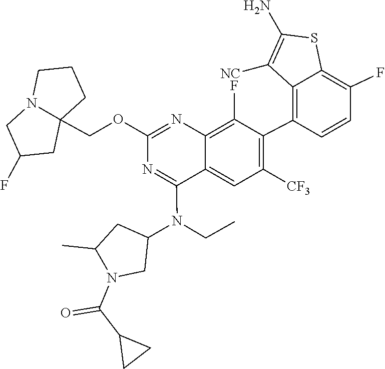 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)C2CC2)C1] | |
| 471 | 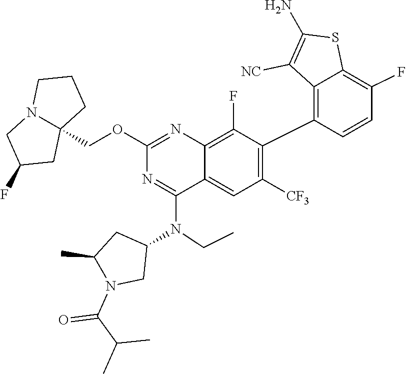 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@H]1C[C@H](C)N(C(=O)C(C)C)C1] | LCMS m/z [M + 1]: 760.28 1H NMR (400 MHz, CD3OD) δ 8.24 (s, 1H), 7.25- 7.18 (m, 1H), 7.05-6.96 (m, 1H), 5.40 (s, 0.5H), 5.36- 5.20 (m, 1.5H), 4.44-4.25 (m, 3H), 4.16-3.58 (m, 5H), 3.49-3.35 (m, 1H), 3.13-3.03 (m, 1H), 2.81-2.58 (m, 2H), 2.46-2.00 (m, 7H), 1.96-1.85 (m, 1H), 1.47- 1.38 (m, 3H), 1.36-1.30 (m, 3H), 1.17-1.08 (m, 6H). |
| 471a | 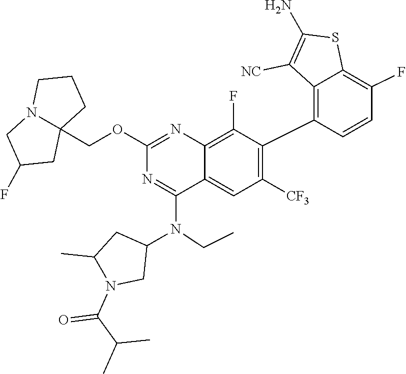 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)C(C)C)C1] | |
| 472 | 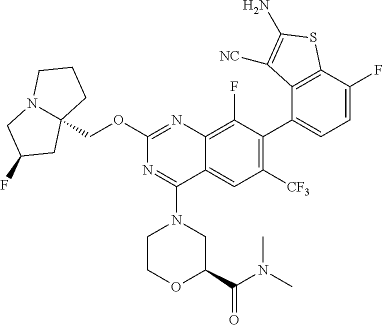 [SMILES: CN(C)C(=O)[C@@H]1CN(c2nc(OC[C@@]34CCCN3C[C@H](F)C4)nc3c(F)c(-c4ccc(F)c5sc(N)c(C#N)c45)c(C(F)(F)F)cc23)CCO1] | LCMS m/z [M + 1]: 720.1 1H NMR (400 MHz, CD3OD) δ8.23 (d, J = 5.2 Hz, 1H), 7.23-7.19 (m, 1H), 7.01 (t, J = 8.8 Hz, 1H), 5.33 (d, J = 53.2 Hz, 1H), 4.68-4.63 (m, 1H), 4.54-4.45 (m, 1H), 4.40-4.27 (m, 3H), 4.04-4.01 (m, 1H), 3.88-3.61 (m, 3H), 3.42-3.32 (m, 3H), 3.18 (d, J = 13.2 Hz, 3H), 3.14-3.03 (m, 1H), 2.97 (d, J = 13.2 Hz, 3H), 2.42- 1.93 (m, 6H). |
| 473 | 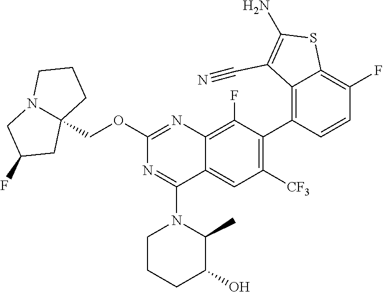 [SMILES: C[C@H]1[C@H](O)CCCN1c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 677.1 1H NMR (400 MHz, CD3OD) δ8.21-8.10 (m, 1H), 7.22-7.19 (m, 1H), 7.02 (t, J = 8.8 Hz, 1H), 5.54 (d, J = 44.8 Hz, 1H), 4.71-4.32 (m, 4H), 4.01-3.80 (m, 3H), 3.51-3.39 (m, 3H), 2.68-2.50 (m, 2H), 2.39-2.30 (m, 3H), 2.15-1.83 (m, 5H), 1.64-1.29 (m, 4H). |
| 473a | 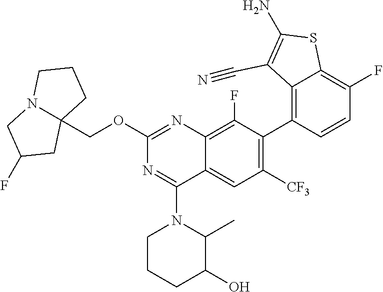 [SMILES: CC1C(O)CCCN1c1nc(OCC23CCCN2CC(F)C3)nc2c(F)c(-c3ccc(F)c4sc(N)c(C#N)c34)c(C(F)(F)F)cc12] | |
| 474 | 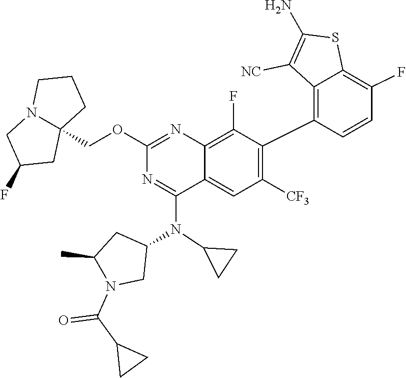 [SMILES: C[C@H]1C[C@H](N(c2nc(OC[C@@]34CCCN3C[C@H](F)C4)nc3c(F)c(-c4ccc(F)c5sc(N)c(C#N)c45)c(C(F)(F)F)cc23)C2CC2)CN1C(=O)C1CC1] | LCMS m/z [M + 1]: 770.3 1H NMR (400 MHz, CD3OD) δ 8.69 (s, 1H), 7.22 (s, 1H), 7.01 (s, 1H), 5.41 (s, 0.54H), 5.27 (s, 0.50H), 5.25-4.95 (m, 2H), 4.60 (s, 1H), 4.42-4.05 (m, 4H), 3.79 (s, 1H), 3.48 (s, 2H), 3.17-2.85 (m, 3H), 2.41- 1.78 (m, 8H), 1.45-1.29 (m, 3H), 1.03-0.73 (m, 7H). |
| 474a | 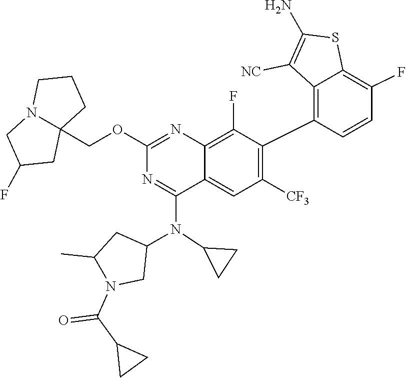 [SMILES: CC1CC(N(c2nc(OCC34CCCN3CC(F)C4)nc3c(F)c(-c4ccc(F)c5sc(N)c(C#N)c45)c(C(F)(F)F)cc23)C2CC2)CN1C(=O)C1CC1] | |
| 475 | 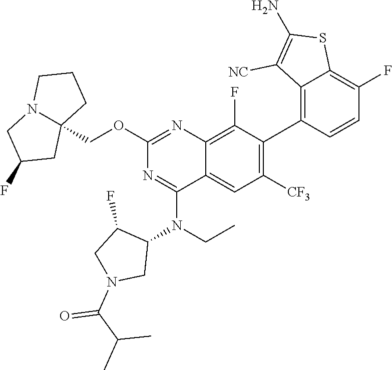 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@@H]1CN(C(=O)C(C)C)C[C@@H]1F] | LCMS m/z [M + 1]: 764.20 1H NMR (400 MHz, CD3OD) δ 8.32 (s, 1H), 7.23- 7.22 (m, 1H), 7.01 (t, 1H), 5.82 (s, 0.3H), 5.75 (s, 0.3H), 5.68 (s, 0.3H), 5.62 (s, 0.3H), 5.40 (s, 0.5H), 5.27 (s, 0.5H), 5.13-5.03 (m, 1H), 4.38-4.22 (m, 3H), 4.17-3.96 (m, 4H), 3.91-3.73 (m, 2H), 3.43-3.33 (m, 3H), 3.12-3.09 (m, 1H), 2.95-2.73 (m, 2H), 2.44-2.13 (m, 4H), 2.08-1.89 (m, 3H), 1.43-1.28 (m, 3H), 1.19- 1.09 (m, 6H). |
| 475a | 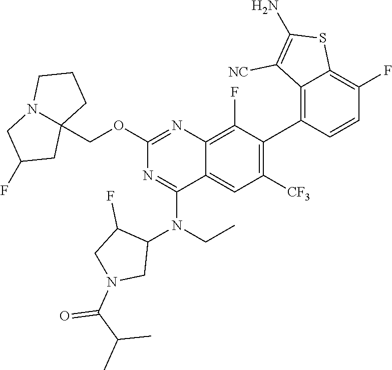 [SMILES: CCN(c1nc(OCC23CCCN2CC(F)C3)nc2c(F)c(-c3ccc(F)c4sc(N)c(C#N)c34)c(C(F)(F)F)cc12)C1CN(C(=O)C(C)C)CC1F] | |
| 476 | 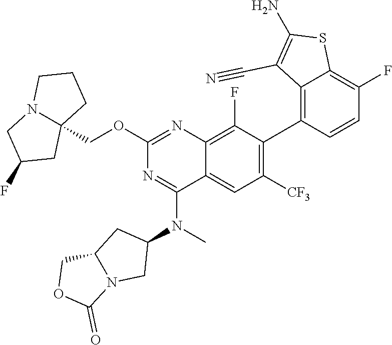 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@@H]1C[C@H]2COC(=O)N2C1] | LCMS m/z [M + 1]: 718.2 1H NMR (400 MHz, CD3OD) δ 8.31 (d, J = 3.2 Hz, 1H), 7.24-7.21 (m, 1H), 7.05-7.00 (m, 1H), 5.46 (s, 0.56H), 5.33 (s, 1.6H), 4.69-4.63 (m, 1H), 4.52-4.45 (m, 2H), 4.38-4.25 (m, 3H), 3.58-3.45 (m, 6H), 3.30- 3.25 (m, 1H), 3.17-3.14 (m, 1H), 2.69-2.49 (m, 1H), 2.47-2.09 (m, 6H), 2.07-1.99 (m, 1H). |
| 477 | 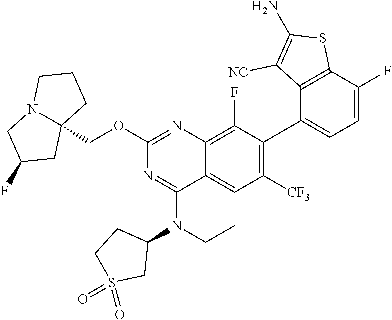 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@@H]1CCS(=O)(=O)C1] | LCMS m/z [M + 1]: 725.17 1H NMR (400 MHz, CD3OD) δ 8.22 (s, 1H), 7.25- 7.16 (m, 1H), 7.02 (t, J = 8.9 Hz, 1H), 5.39 (s, 0.5H), 5.25 (s, 0.5H), 4.99-4.89 (m, 1H), 4.37-4.24 (m, 2H), 3.93-3.81 (m, 2H), 3.80-3.64 (m, 2H), 3.58-3.47 (m, 1H), 3.39-3.32 (m, 1H), 3.26-3.13 (m, 3H), 3.07-2.99 (m, 1H), 2.85-2.71 (m, 1H), 2.69-2.56 (m, 1H), 2.44- 2.28 (m, 1H), 2.24-2.10 (m, 2H), 2.04-1.87 (m, 3H), 1.61-1.49 (m, 3H). |
| 478 | 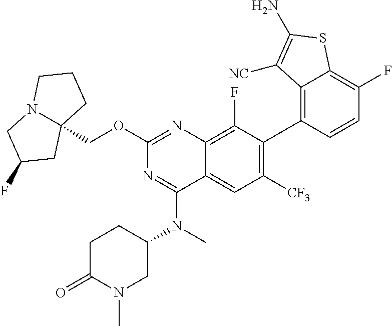 [SMILES: CN1C[C@@H](N(C)c2nc(OC[C@@]34CCCN3C[C@H](F)C4)nc3c(F)c(-c4ccc(F)c5sc(N)c(C#N)c45)c(C(F)(F)F)cc23)CCC1=O] | LCMS m/z [M + 1]: 704.1 1H NMR (400 MHz, CD3OD) δ 8.34 (s, 1H), 7.25- 7.18 (m, 1H), 7.01 (m, 1H), 5.44 (s, 0.5H), 5.31 (s, 0.5H), 5.13 (s, 1H), 4.39 (m, 2H), 3.68 (m, 2H), 3.48 (s, 4H), 3.45 (s, 2H), 3.15 (m, 1H), 3.00 (s, 3H), 2.64-2.29 (m, 5H), 2.21 (s, 2H), 2.10 (s, 2H), 1.95 (s, 1H). |
| 479 | 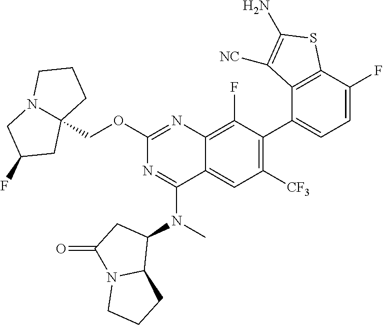 [SMILES: CN(c1nc(OC[C@@]23CCCN2C[C@H](F)C3)nc2c(F)c(-c3ccc(F)c4sc(N)c(C#N)c34)c(C(F)(F)F)cc12)[C@@H]1CC(=O)N2CCC[C@H]12] | LCMS m/z [M + 1]: 716.1 1H NMR (400 MHz, CD3OD) δ 8.33 (s, 1H), 7.25- 7.19 (m, 1H), 7.01 (t, 1H), 5.42 (s, 0.5H), 5.29 (s, 0.5H), 5.24-5.16 (m, 1H), 4.44-4.16 (m, 4H), 3.62- 3.54 (m, 3H), 3.49-3.43 (m, 1H), 3.42-3.34 (m, 2H), 3.21-3.07 (m, 3H), 2.96-2.89 (m, 1H), 2.43-1.88 (m, 10H) |
| 479a | 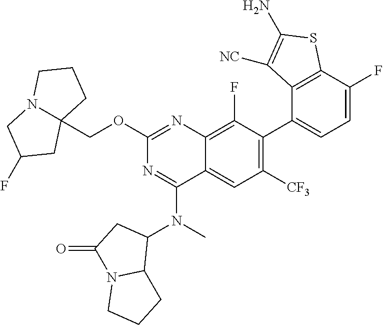 [SMILES: CN(c1nc(OCC23CCCN2CC(F)C3)nc2c(F)c(-c3ccc(F)c4sc(N)c(C#N)c34)c(C(F)(F)F)cc12)C1CC(=O)N2CCCC12] | |
| 480 | 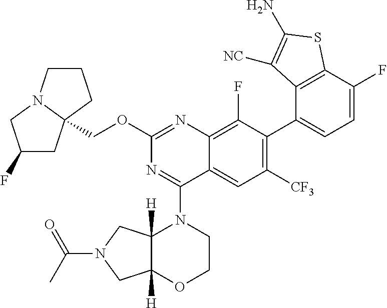 | LCMS m/z [M + 1]: 732 1H-NMR (400 MHz, CD3OD-d): 8.16-8.19 (m, 1 H), 7.19-7.27 (m, 1 H), 7.01 (t, J = 8.8 Hz, 1 H), 5.18-5.40 (m, 2 H), 4.31-4.26 (m, 4 H), 3.89-4.03 (m, 3 H), 3.57-3.84 (m, 4 H), 3.35-3.43 (m, 3 H), 3.09 (s, 1 H), 1.93-2.39 (m, 9H). |
| 481 | 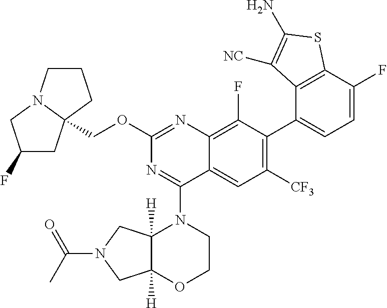 | LCMS m/z [M + 1]: 732 1H-NMR (400 MHz, CD3OD-d): 8.48 (s, 1 H), 8.21- 8.19 (s, 1 H), 7.04-7.21 (m, 1 H), 6.90-7.01 (m, 1 H), 5.46 (s, 0.5 H), 5.33 (s, 0.5 H), 5.21-5.33 (m, 1 H), 4.17-4.38 (m, 4 H), 3.94-4.00 (m, 3 H), 3.77-3.94 (m, 2 H), 3.66-3.77 (m, 5 H), 3.50-3.31 (m, 1 H), 1.98- 2.25 (m, 9H). |
| 481a | 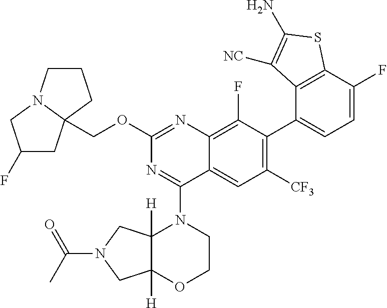 | |
| 482 | 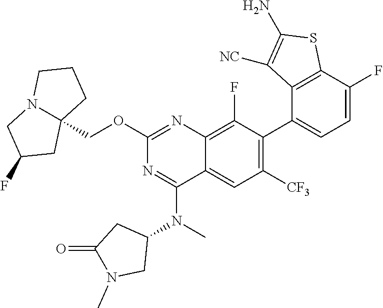 | LCMS m/z [M + 1]: 704.1 1H NMR (400 MHz, CD3OD) δ 8.16 (s, 1H), 7.19- 7.22 (m, 1H), 7.01 (t, 1H), 5.42 (s, 0.5H), 5.29 (s, 0.5H), 4.68-4.76 (m, 1H), 4.12-4.23 (m, 2H), 3.75- 3.90 (m, 4H), 3.47 (s, 1H), 2.95-3.07 (m, 2H), 2.79- 2.93 (m, 4H), 2.13-2.40 (m, 4H), 1.86-2.05 (m, 4H), 1.51-1.55 (m, 3H). |
| 482a | 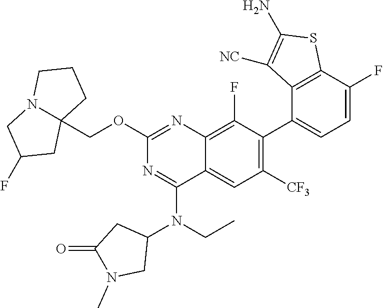 [SMILES: CCN(c1nc(OCC23CCCN2CC(F)C3)nc2c(F)c(-c3ccc(F)c4sc(N)c(C#N)c34)c(C(F)(F)F)cc12)C1CC(=O)N(C)C1] | |
| 483 | 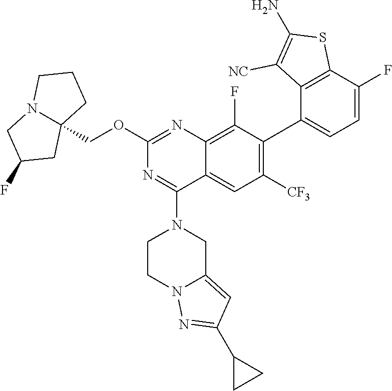 [SMILES: N#Cc1c(N)sc2c(F)ccc(-c3c(C(F)(F)F)cc4c(N5CCn6nc(C7CC7)cc6C5)nc(OC[C@@]56CCCN5C[C@H](F)C6)nc4c3F)c12] | LCMS m/z [M + 1]: 725.22 1H NMR (400 MHz, CD3OD) δ 8.21 (s, 1H), 7.25- 7.19 (m, 1H), 7.05-6.98 (m, 1H), 5.9 (s, 1H), 5.42 (s, 0.5H), 5.29 (s, 0.5H), 5.15 (s, 2H), 4.61-4.24 (m, 6H), 3.56-3.33 (m, 3H), 3.12 (s, 1H), 2.46-2.17 (m, 3H), 2.12-1.83 (m, 4H), 0.97-0.83 (m, 2H), 0.74-0.61 (m, 2H). |
| 483a | 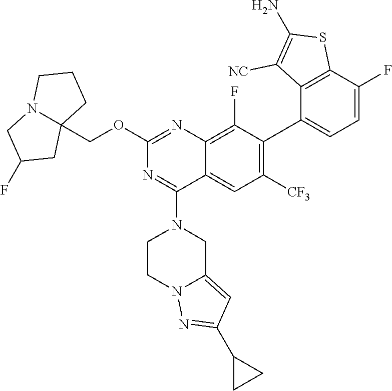 [SMILES: N#Cc1c(N)sc2c(F)ccc(-c3c(C(F)(F)F)cc4c(N5CCn6nc(C7CC7)cc6C5)nc(OCC56CCCN5CC(F)C6)nc4c3F)c12] | |
| 484 | 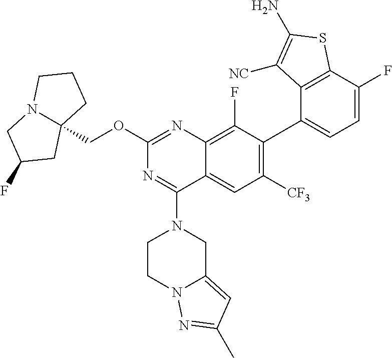 | LCMS m/z [M + 1]: 699.20 1H NMR (400 MHz, CD3OD) δ 8.25 (s, 1H), 7.28- 7.18 (m, 1H), 7.03 (t, J = 8.9 Hz, 1H), 6.02 (s, 1H), 5.52 (s, 0.5H), 5.39 (s, 0.5H), 5.21 (s, 2H), 4.64-4.31 (m, 6H), 3.88-3.45 (m, 4H), 2.62-2.40 (m, 2H), 2.37- 2.28 (m, 1H), 2.26-1.95 (m, 6H). |
| 484a | 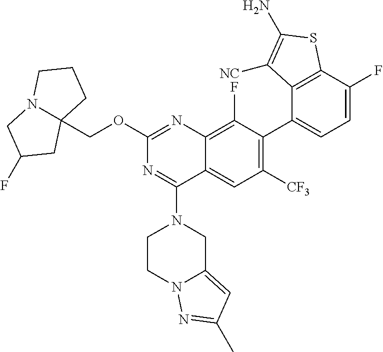 | |
| 485 | 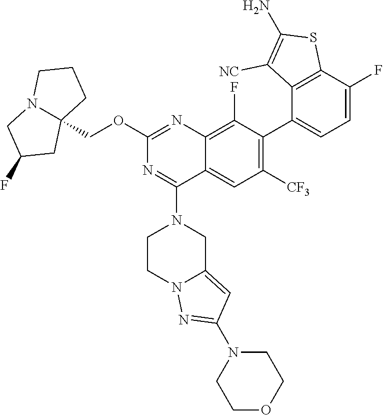 [SMILES: N#Cc1c(N)sc2c(F)ccc(-c3c(C(F)(F)F)cc4c(N5CCn6nc(N7CCOCC7)cc6C5)nc(OC[C@@]56CCCN5C[C@H](F)C6)nc4c3F)c12] | LCMS m/z [M + 1]: 770.24 1H NMR (400 MHz, CD3OD) δ 8.20 (s, 1H), 7.27- 7.18 (m, 1H), 7.02 (t, J = 8.8 Hz, 1H), 5.72 (s, 1H), 5.37 (s, 0.5H), 5.23 (s, 0.5H), 5.12 (s, 2H), 4.52-4.21 (m, 6H), 3.86-3.70 (m, 4H), 3.27-2.97 (m, 8H), 2.38- 2.11 (m, 3H), 2.04-1.88 (m, 3H). |
| 485a | 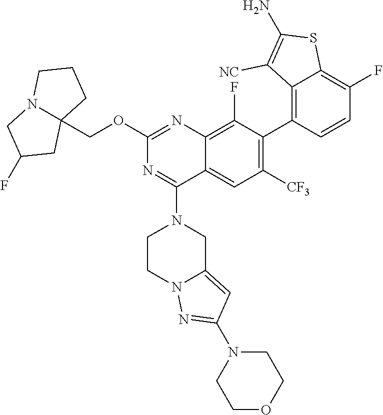 [SMILES: N#Cc1c(N)sc2c(F)ccc(-c3c(C(F)(F)F)cc4c(N5CCn6nc(N7CCOCC7)cc6C5)nc(OCC56CCCN5CC(F)C6)nc4c3F)c12] | |
| 486 | 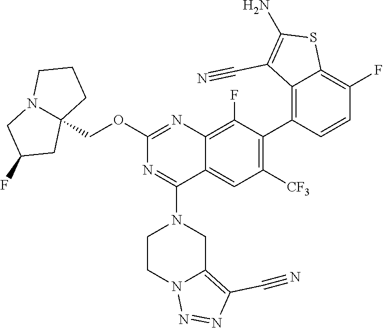 [SMILES: N#Cc1nnn2c1CN(c1nc(OC[C@@]34CCCN3C[C@H](F)C4)nc3c(F)c(-c4ccc(F)c5sc(N)c(C#N)c45)c(C(F)(F)F)cc13)CC2] | LCMS m/z [M + 1]: 711 1H-NMR (400 MHz, CD3OD-d): 8.28 (s, 1 H), 7.25- 7.22 (m, 1 H), 7.03 (t, J = 9.2 Hz, 1 H), 5.47-5.34 (m, 3 H), 4.80-4.78 (m, 2 H), 4.50-4.41 (m, 4 H), 3.57- 3.48 (m, 3 H), 3.20-3.19 (m, 1 H), 2.52-2.25 (m, 3 H), 2.19-2.09 (m, 2H), 2.06-1.95 (m, 1 H). |
| 486a | 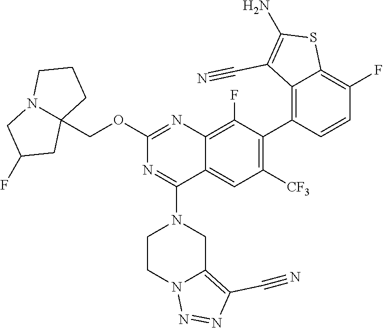 [SMILES: N#Cc1nnn2c1CN(c1nc(OCC34CCCN3CC(F)C4)nc3c(F)c(-c4ccc(F)c5sc(N)c(C#N)c45)c(C(F)(F)F)cc13)CC2] | |
| 487 | 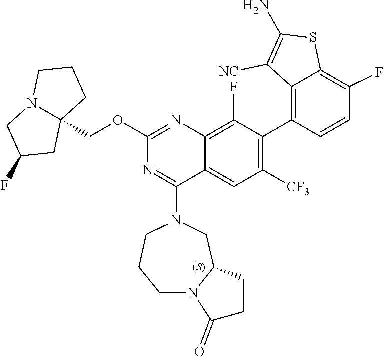 | LCMS m/z [M + 1]: 716.1 1H NMR (400 MHz, CD3OD) δ 8.41 (s, 1H), 8.26 (s, 1H), 7.23-7.20 (m, 1H), 7.02 (t, 1H), 5.55 (s, 0.5H), 5.43 (s, 0.5H), 4.67-4.49 (m, 2H), 4.42-4.34 (d, 1H), 4.30-4.11 (m, 4H), 4.08-4.00 (m, 1H), 3.90-3.68 (m, 3H), 3.40-3.33 (m, 1H), 3.03-2.94 (m, 1H), 2.67-2.42 (m, 4H), 2.40-1.98 (m, 8H), 1.62-1.56 (m, 1H). |
| 487a | 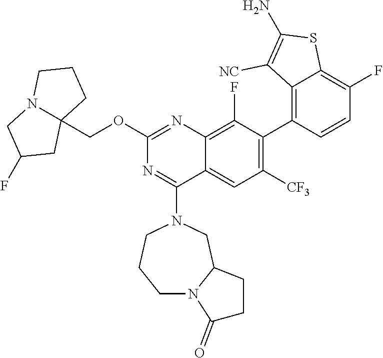 | |
| 488 | 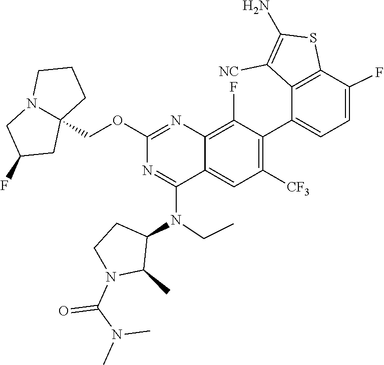 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@@H]1CCN(C(=O)N(C)C)[C@@H]1C] | LCMS m/z [M + 1]: 761.3 1H NMR (400 MHz, CD3OD) δ 8.33 (s, 1H), 7.27- 7.18 (m, 1H), 7.02 (t, 1H), 5.57 (s, 0.5H), 5.44 (s, 0.5H), 5.05-4.99 (m, 1H), 4.90-4.85 (m, 1H), 4.77- 4.67 (m, 1H), 4.59-4.50 (m, 1H), 4.16-4.05 (m, 1H), 3.98-3.64 (m, 5H), 3.42-3.31 (m, 3H), 2.91 (s, 5H), 2.75-2.36 (m, 5H), 2.32-2.08 (m, 3H), 1.59-1.36 (m, 3H), 1.12-0.90 (m, 3H). |
| 488a | 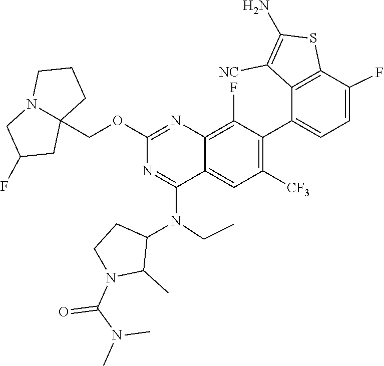 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N(C)C)C1C] | |
| 489 | 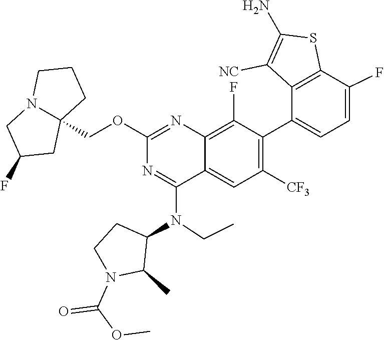 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@@H]1CCN(C(=O)OC)[C@@H]1C] | LCMS m/z [M + 1]: 748.2 1H NMR (400 MHz, CD3OD) δ 8.33 (s, 1H), 7.25- 7.20 (m, 1H), 7.02 (t, 1H), 5.50 (s, 0.5H), 5.36 (s, 0.5H), 4.94-4.87 (m, 2H), 4.80-4.74 (m, 2H), 4.57- 4.48 (m, 2H), 4.09-4.03 (m, 1H), 3.97-3.90 (m, 1H), 3.71 (s, 3H), 3.54-3.41 (m, 3H), 3.23-3.15 (m, 1H), 2.56-2.37 (m, 4H), 2.19-1.95 (m, 4H), 1.48-1.40 (m, 3H), 1.04-0.94 (m, 3H). |
| 489a | 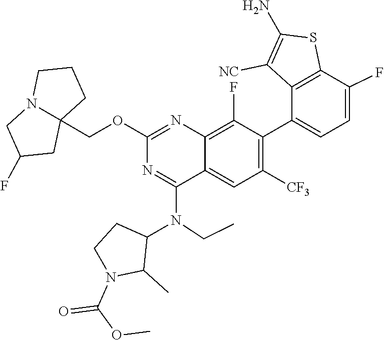 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC)C1C] | |
| 490 | 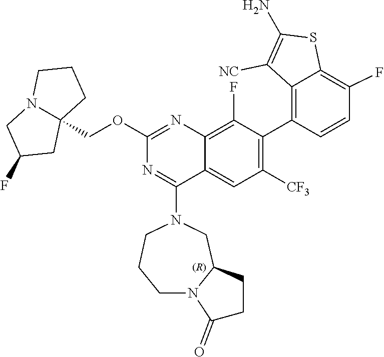 | LCMS m/z [M + 1]: 716.1 1H NMR (400 MHz, CD3OD) δ 8.22 (s, 1H), 7.21 (s, 1H), 7.01 (t, 1H), 5.40 (s, 0.5H), 5.27 (s, 0.5H), 4.32- 4.30 (m, 4H), 4.16 (s, 2H), 4.07 (m, 1H), 3.47 (m, 4H), 3.08-2.99 (m, 2H), 2.44 (t, 2H), 2.30-2.16 (m, 6H), 2.03-1.92 (m, 4H). |
| 491 | 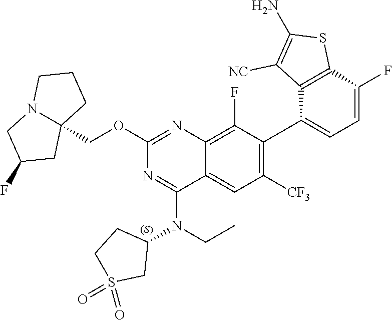 | LCMS m/z [M + 1]: 725.17 1H NMR (400 MHz, CD3OD) δ 8.25 (s, 1H), 7.25- 7.19 (m, 1H), 7.06-6.99 (m, 1H), 5.64 (s, 0.5H), 5.52 (s, 0.5H), 4.92-4.85 (m, 1H), 4.76-4.64 (m, 2H), 4.11-3.82 (m, 6H), 3.68-3.60 (m, 1H), 3.57-3.42 (m, 2H), 3.27-3.18 (m, 1H), 2.96-2.69 (m, 2H), 2.69-2.53 (m, 2H), 2.49-2.40 (m, 1H), 2.37-2.27 (m, 2H), 2.23- 2.11 (m, 1H), 1.63-1.55 (m, 3H). |
| 492 | 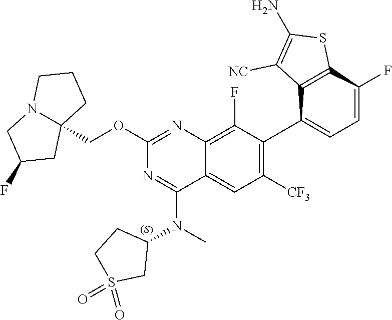 | LCMS m/z [M + 1]: 725.17 1H NMR (400 MHz, CD3OD) δ 8.25 (s, 1H), 7.25- 7.19 (m, 1H), 7.06-6.99 (m, 1H), 5.65 (s, 0.5H), 5.52 (s, 0.5H), 4.94-4.86 (m, 1H), 4.75-4.64 (m, 2H), 4.12-3.82 (m, 6H), 3.68-3.58 (m, 1H), 3.57-3.42 (m, 2H), 3.26-3.18 (m, 1H), 2.96-2.85 (m, 1H), 2.81-2.67 (m, 1H), 2.66-2.55 (m, 2H), 2.51-2.42 (m, 1H), 2.39- 2.27 (m, 2H), 2.22-2.10 (m, 1H), 1.63-1.55 (m, 3H). |
| 494 | 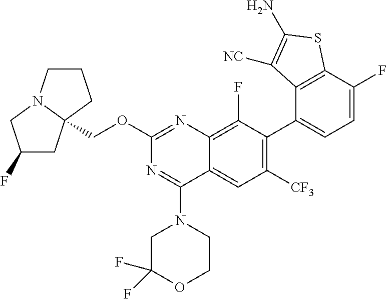 | LCMS m/z [M + 1]: 732.2 1H NMR (400 MHz, CD3OD) δ 8.29 (s, 1H), 7.20 (d, J = 5.3 Hz, 1H), 7.02 (d, J = 9.1 Hz, 1H), 5.36 (d, J = 53.2 Hz, 1H), 5.12 (dt, J = 16.9, 8.2 Hz, 1H), 4.37 (dd, J = 21.4, 5.8 Hz, 2H), 3.43 (s, 6H), 3.14 (s, 4H), 2.98 (s, 3H), 2.58-1.66 (m, 13H). |
| 494a | 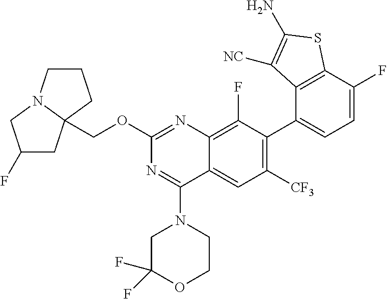 | |
| 496 | 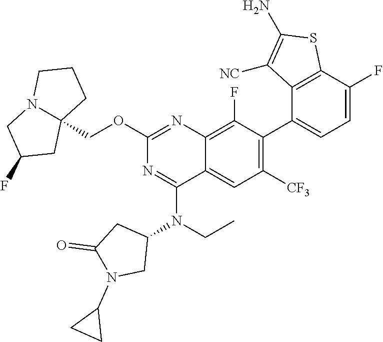 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@H]1CC(=O)N(C2CC2)C1] | LCMS m/z [M + 1]: 730.4 1H NMR (400 MHz, CD3OD) δ 8.169 (s, 1H), 7.209 (t, 1H), 7.014 (t, 1H), 5.496 (s, 0.5H), 5.366 (s, 0.5H), 4.649 (s, 1H), 4.422-4.279 (m, 2H), 3.874- 3.805 (m, 2H), 3.734-3.674 (m, 1H), 3.552-3.433 (m, 2H), 3.224-3.160 (m, 1H), 2.975-3.753 (m, 3H), 2.582-2.227 (m, 4H), 2.200-1.871 (m, 4H), 1.534 (t, 3H), 0.922-0.682 (m, 4H). |
| 496a | 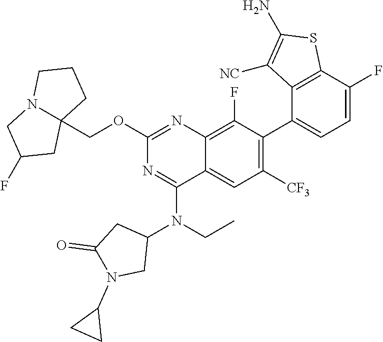 [SMILES: CCN(c1nc(OCC23CCCN2CC(F)C3)nc2c(F)c(-c3ccc(F)c4sc(N)c(C#N)c34)c(C(F)(F)F)cc12)C1CC(=O)N(C2CC2)C1] | |
| 497 | 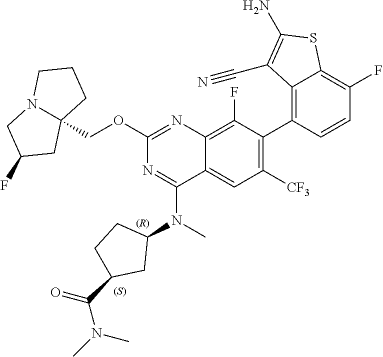 | LCMS m/z [M + 1]: 732.2 1H NMR (400 MHz, CD3OD) δ 8.29 (s, 1H), 7.20 (d, J = 5.3 Hz, 1H), 7.02 (d, J = 9.1 Hz, 1H), 5.36 (d, J = 53.2 Hz, 1H), 5.12 (dt, J = 16.9, 8.2 Hz, 1H), 4.37 (dd, J = 21.4, 5.8 Hz, 2H), 3.43 (s, 6H), 3.14 (s, 4H), 2.98 (s, 3H), 2.58-1.66 (m, 13H). |
| 497a | 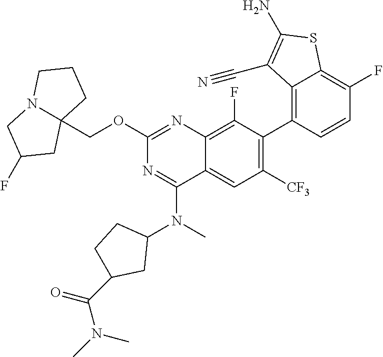 [SMILES: CN(C)C(=O)C1CCC(N(C)c2nc(OCC34CCCN3CC(F)C4)nc3c(F)c(-c4ccc(F)c5sc(N)c(C#N)c45)c(C(F)(F)F)cc23)C1] | |
| 498 | 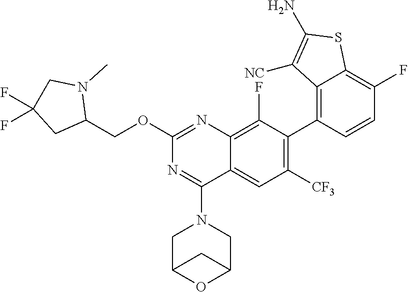 [SMILES: CN1CC(F)(F)CC1COc1nc(N2CC3CC(C2)O3)c2cc(C(F)(F)F)c(-c3ccc(F)c4sc(N)c(C#N)c34)c(F)c2n1] | LCMS m/z [M + 1]: 653.2 1H-NMR (400 MHz, CD3OD-d): 8.61 (d, J = 6.0 Hz, 1 H), 7.05-6.99 (m, 1 H), 4.85-4.84 (m, 2 H), 4.57- 4.48 (m , 4 H), 4.40 (s, 1 H), 4.03 (s, 1 H), 3.32-3.31 (m, 2 H), 3.07 (s, 1 H), 2.80-2.67 (m, 2 H), 2.58-2.54 (m, 1H), 2.50 (d, J = 6.0 Hz, 1 H), 2.29 (s, 1 H), 1.98 (t, J = 7.6 Hz, 1 H). |
| 498a | 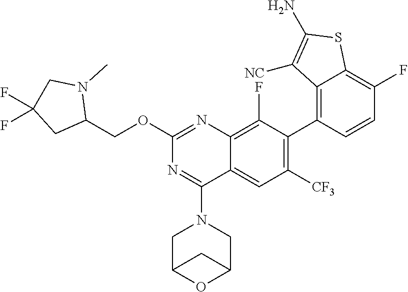 [SMILES: CN1CC(F)(F)CC1COc1nc(N2CC3CC(C2)O3)c2cc(C(F)(F)F)c(-c3ccc(F)c4sc(N)c(C#N)c34)c(F)c2n1] | |
| 499 | 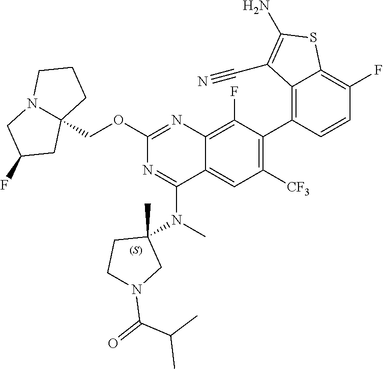 | LCMS m/z [M + 1]: 746.2 1H NMR (400 MHz, CD3OD) δ 8.51 (s, 0.59H), 8.25 (s, 1H), 7.20 (s, 1H), 7.03-6.98 (m, 1H), 5.46- 5.27 (m, 1H), 4.57-4.20 (m, 3H), 3.87-3.55 (m, 3H), 3.54-3.34 (m, 5H), 3.18-3.04 (m, 1H), 2.88-2.69 (m, 1H), 2.68-1.81 (m, 9H), 1.76-1.57 (m, 3H), 1.24-0.95 (m, 6H). |
| 499a | 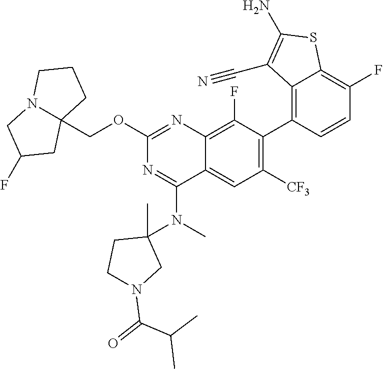 [SMILES: CC(C)C(=O)N1CCC(C)(N(C)c2nc(OCC34CCCN3CC(F)C4)nc3c(F)c(-c4ccc(F)c5sc(N)c(C#N)c45)c(C(F)(F)F)cc23)C1] | |
| 500 | 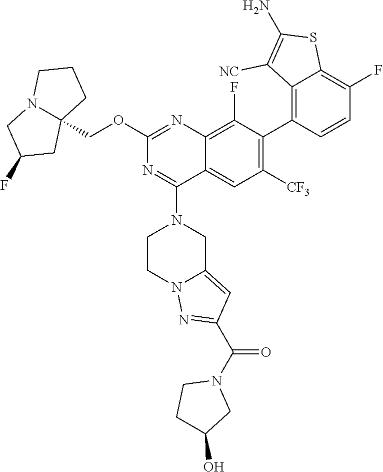 | LCMS m/z [M + 1]: 798.1 1H NMR (400 MHz, CD3OD) δ 8.23 (s, 1H), 7.22 (t, 1H), 7.01 (t, 1H), 8.65 (s, 1H), 5.39 (s, 0.5H), 5.25 (s, 2.5H), 4.36-4.53 (m, 6H), 4.29-4.33 (m, 1H), 3.93- 4.15 (m, 2H), 3.70-3.74 (m, 1H), 3.60-3.66 (m, 1H), 3.26 (s, 1H), 3.02-3.08 (m, 1H), 1.94-2.40 (m, 10H). |
| 501 | 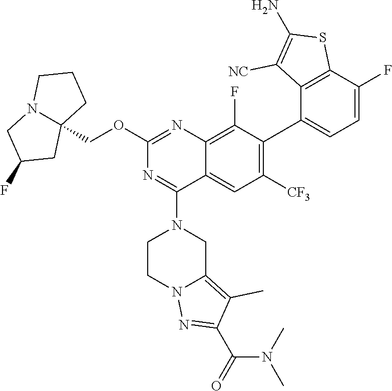 [SMILES: Cc1c(C(=O)N(C)C)nn2c1CN(c1nc(OC[C@@]34CCCN3C[C@H](F)C4)nc3c(F)c(-c4ccc(F)c5sc(N)c(C#N)c45)c(C(F)(F)F)cc13)CC2] | LCMS m/z [M + 1]: 770.24 1H NMR (400 MHz, CD3OD) δ 8.23 (s, 1H), 7.26- 7.18 (m, 1H), 7.05-6.97 (m, 1H), 5.36 (s, 0.5H), 5.23 (s, 0.5H), 5.11 (s, 2H), 4.50-4.23 (m, 6H), 3.26-2.94 (m, 10H), 2.39-2.11 (m, 3H), 2.09-1.85 (m, 6H). |
| 501a | 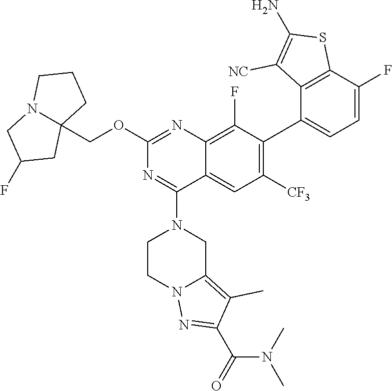 [SMILES: Cc1c(C(=O)N(C)C)nn2c1CN(c1nc(OCC34CCCN3CC(F)C4)nc3c(F)c(-c4ccc(F)c5sc(N)c(C#N)c45)c(C(F)(F)F)cc13)CC2] | |
| 502 | 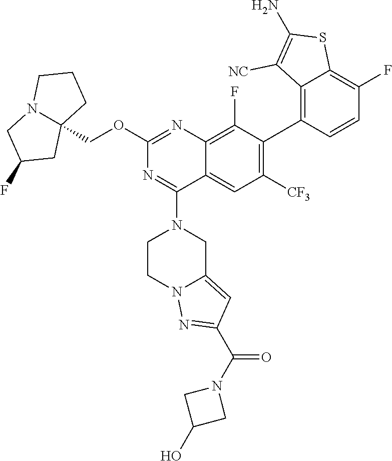 [SMILES: N#Cc1c(N)sc2c(F)ccc(-c3c(C(F)(F)F)cc4c(N5CCn6nc(C(=O)N7CC(O)C7)cc6C5)nc(OC[C@@]56CCCN5C[C@H](F)C6)nc4c3F)c12] | LCMS m/z [M + 1]: 784.2 1H NMR (400 MHz, CD3OD) δ 8.27 (s, 1H), 7.21- 7.25 (m, 1H), 7.02 (t, 1H), 6.64 (s, 1H), 5.55 (s, 0.5H), 5.42 (s, 0.5H), 5.28 (s, 2H), 4.77-4.80 (m, 1H), 4.47-4.61 (m, 7H), 4.32-4.38 (m, 2H), 3.88-3.92 (m, 1H), 3.72-3.80 (m, 3H), 3.34-3.38 (m, 1H), 2.53- 2.65 (m, 2H), 2.33-2.47 (m, 1H), 2.23-2.27 (m, 2H), 2.09 (s, 1H). |
| 502a | 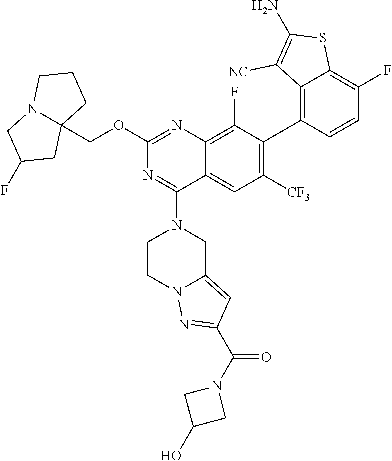 [SMILES: N#Cc1c(N)sc2c(F)ccc(-c3c(C(F)(F)F)cc4c(N5CCn6nc(C(=O)N7CC(O)C7)cc6C5)nc(OCC56CCCN5CC(F)C6)nc4c3F)c12] | |
| 503 | 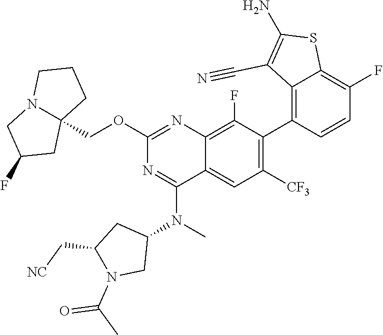 [SMILES: CC(=O)N1C[C@@H](N(C)c2nc(OC[C@@]34CCCN3C[C@H](F)C4)nc3c(F)c(-c4ccc(F)c5sc(N)c(C#N)c45)c(C(F)(F)F)cc23)C[C@H]1CC#N] | LCMS m/z [M + 1]: 743.0 1H NMR (400 MHz, CD3OD) δ 8.36 (s, 1H), 7.22 (t, 1H), 7.02 (t, 1H), 5.54 (s, 0.5H), 5.41 (s, 0.5H), 5.25 (t, 1H), 4.65-4.50 (m, 2H), 4.30 (d, 1H), 4.14 (t, 1H), 3.81-3.71 (m, 4H), 3.54 (s, 3H), 3.42-3.33 (m, 1H), 3.29-2.25 (m, 1H), 2.99 (d, 1H), 2.80-2.73 (m, 1H), 2.65-2.46 (m, 2H), 2.35-2.07 (m, 8H). |
| 503a | 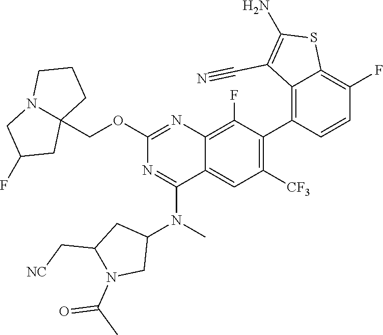 [SMILES: CC(=O)N1CC(N(C)c2nc(OCC34CCCN3CC(F)C4)nc3c(F)c(-c4ccc(F)c5sc(N)c(C#N)c45)c(C(F)(F)F)cc23)CC1CC#N] | |
| 504 | 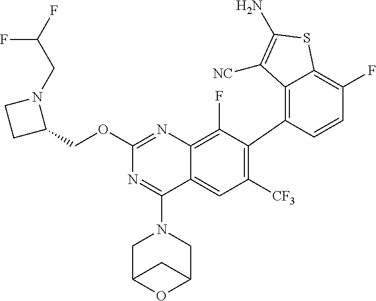 [SMILES: N#Cc1c(N)sc2c(F)ccc(-c3c(C(F)(F)F)cc4c(N5CC6CC(C5)O6)nc(OC[C@@H]5CCN5CC(F)F)nc4c3F)c12] | LCMS m/z [M + 1]: 743.0 1H NMR (400 MHz, CD3OD) δ 8.36 (s, 1H), 7.22 (t, 1H), 7.02 (t, 1H), 5.54 (s, 0.5H), 5.41 (s, 0.5H), 5.25 (t, 1H), 4.65-4.50 (m, 2H), 4.30 (d, 1H), 4.14 (t, 1H), 3.81-3.71 (m, 4H), 3.54 (s, 3H), 3.42-3.33 (m, 1H), 3.29-2.25 (m, 1H), 2.99 (d, 1H), 2.80-2.73 (m, 1H), 2.65-2.46 (m, 2H), 2.35-2.07 (m, 8H). |
| 504a | 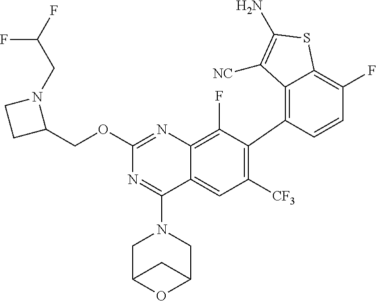 [SMILES: N#Cc1c(N)sc2c(F)ccc(-c3c(C(F)(F)F)cc4c(N5CC6CC(C5)O6)nc(OCC5CCN5CC(F)F)nc4c3F)c12] | |
| 505 | 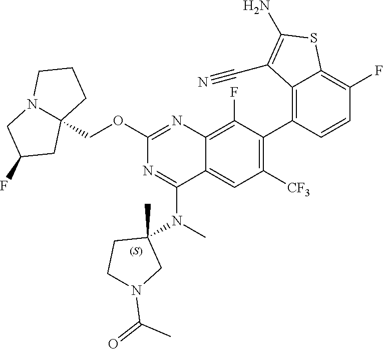 | LCMS m/z [M + 1]: 718.1 1H NMR (400 MHz, CD3OD) δ = 8.26 (s, 1H), 7.21 (s, 1H), 7.02 (d, J = 9.1, 1H), 5.39 (d, J = 29.9, 1H), 4.90 (m, 1H), 4.44 (m, 3H), 3.48 (m, 7H), 3.13 (m, 2H), 2.08 (m, 11H), 1.70 (s, 3H). |
| 505a | 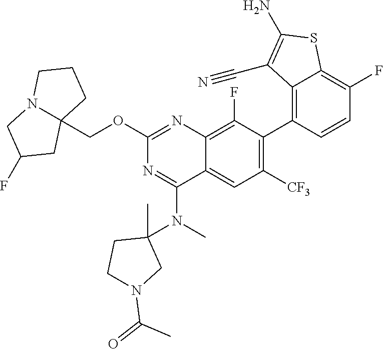 [SMILES: CC(=O)N1CCC(C)(N(C)c2nc(OCC34CCCN3CC(F)C4)nc3c(F)c(-c4ccc(F)c5sc(N)c(C#N)c45)c(C(F)(F)F)cc23)C1] | |
| 506 | 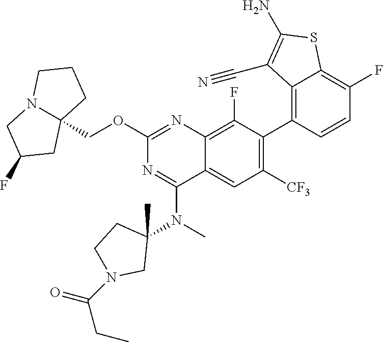 [SMILES: CCC(=O)N1CC[C@](C)(N(C)c2nc(OC[C@@]34CCCN3C[C@H](F)C4)nc3c(F)c(-c4ccc(F)c5sc(N)c(C#N)c45)c(C(F)(F)F)cc23)C1] | LCMS m/z [M + 1]: 732.1 1H NMR (400 MHz, CD3OD) δ 8.43 (s, 1H), 8.29- 8.30 (m, 1H), 7.21-7.22 (m, 1H), 7.01 (t, J = 8.8 Hz, 1H), 5.54-5.56 (m, 1H), 4.40-4.54 (m, 3H), 3.45-3.51 (m, 10H), 2.13-2.62 (m, 10H), 1.64-1.66 (m, 3H), 1.13-1.16 (m, 3H) |
| 506a | 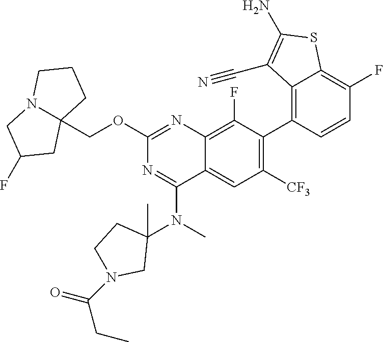 [SMILES: CCC(=O)N1CCC(C)(N(C)c2nc(OCC34CCCN3CC(F)C4)nc3c(F)c(-c4ccc(F)c5sc(N)c(C#N)c45)c(C(F)(F)F)cc23)C1] | |
| 507 | 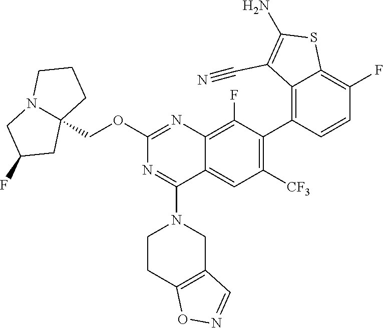 [SMILES: N#Cc1c(N)sc2c(F)ccc(-c3c(C(F)(F)F)cc4c(N5CCc6oncc6C5)nc(OC[C@@]56CCCN5C[C@H](F)C6)nc4c3F)c12] | LCMS m/z [M + 1]: 686.1 1H-NMR (400 MHz, CD3OD-d): 8.34 (s, 1 H), 8.23 (s, 1 H), 7.22 (s, 1 H), 7.02 (t, J = 9.6 Hz, 1 H), 5.43- 5.30 (m, 1 H), 4.97 (s, 2 H), 4.43-4.23 (m, 4 H), 3.48- 3.38 (m, 3 H), 3.22-3.13 (m, 3 H), 2.47-2.21 (m, 3H), 2.08 (s, 2 H), 1.97 (s, 1 H). |
| 507a | 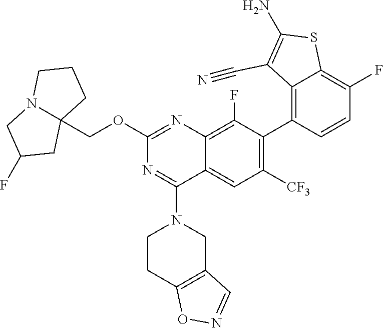 [SMILES: N#Cc1c(N)sc2c(F)ccc(-c3c(C(F)(F)F)cc4c(N5CCc6oncc6C5)nc(OCC56CCCN5CC(F)C6)nc4c3F)c12] | |
| 508 | 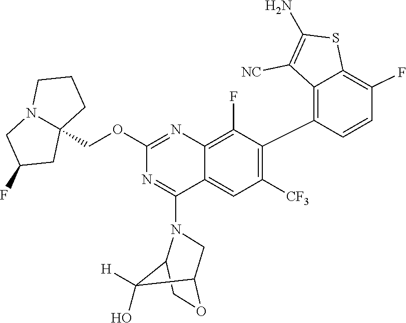 [SMILES: N#Cc1c(N)sc2c(F)ccc(-c3c(C(F)(F)F)cc4c(N5CC6OCC5C6O)nc(OC[C@@]56CCCN5C[C@H](F)C6)nc4c3F)c12] | LCMS m/z [M + 1]: 677.0 1H NMR (400 MHz, CD3OD) δ 8.26 (s, 1H), 7.20 (s, 1H), 7.03-6.98 (m, 1H), 5.38 (s, 0.61H), 5.24 (s, 0.62H), 5.08 (d, J = 14.8 Hz, 1H), 4.94 (s, 1H), 4.40- 4.31 (m, 3H), 4.14-4.01 (m, 3H), 3.33-3.10 (m, 2H), 3.04 (s, 1H), 2.39-1.90 (m, 6H). |
| 508a | 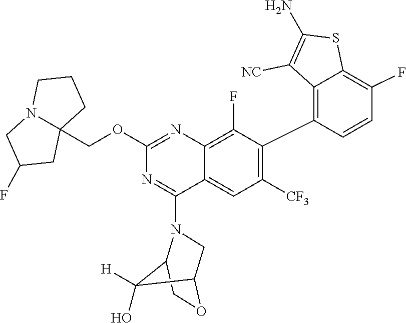 [SMILES: N#Cc1c(N)sc2c(F)ccc(-c3c(C(F)(F)F)cc4c(N5CC6OCC5C6O)nc(OCC56CCCN5CC(F)C6)nc4c3F)c12] | |
| 509 | 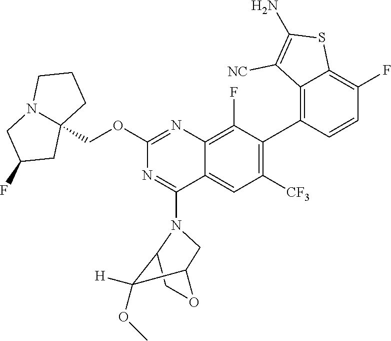 [SMILES: COC1C2CN(c3nc(OC[C@@]45CCCN4C[C@H](F)C5)nc4c(F)c(-c5ccc(F)c6sc(N)c(C#N)c56)c(C(F)(F)F)cc34)C1CO2] | LCMS m/z [M + 1]: 691.0 1H NMR (400 MHz, CD3OD) δ 8.25 (s, 1H), 7.20 (s, 1H), 7.03-7.00 (m, 1H), 5.37 (s, 0.57H), 5.25 (d, J = 14.8 Hz, 1.5H), 4.60 (s, 1H), 4.39-4.04 (m, 7H), 3.53 (s, 3H), 3.02 (s, 1H), 2.34-2.16 (m, 6H). |
| 509a | 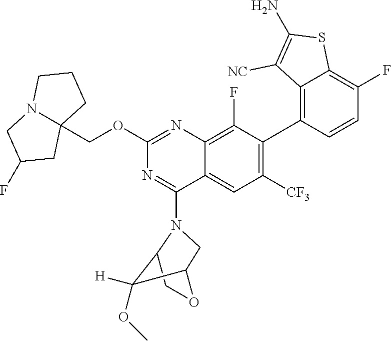 [SMILES: COC1C2CN(c3nc(OCC45CCCN4CC(F)C5)nc4c(F)c(-c5ccc(F)c6sc(N)c(C#N)c56)c(C(F)(F)F)cc34)C1CO2] | |
| 510 | 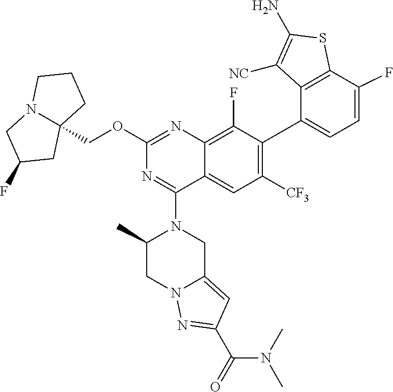 [SMILES: C[C@@H]1Cn2nc(C(=O)N(C)C)cc2CN1c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 770.0 1HNMR (400 MHz, CD3OD) δ 8.21 (s, 1H), 7.21 (s, 1H), 7.06-6.96 (m, 1H), 8.54 (s, 1H), 5.42-5.30 (m, 2H), 5.22-5.12 (m, 1H), 5.02-4.92 (m, 1H), 4.67-4.55 (m, 1H), 4.38-4.13 (m, 3H), 3.51-3.34 (m, 2H), 3.26- 3.00 (m, 8H), 2.33-2.09 (m, 3H), 2.03-1.84 (m, 3H), 1.45-1.28 (m, 3H). |
| 510a | 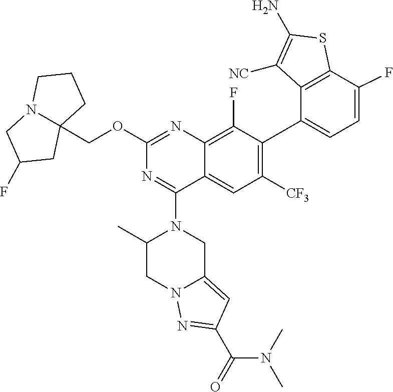 [SMILES: CC1Cn2nc(C(=O)N(C)C)cc2CN1c1nc(OCC23CCCN2CC(F)C3)nc2c(F)c(-c3ccc(F)c4sc(N)c(C#N)c34)c(C(F)(F)F)cc12] | |
| 511 | 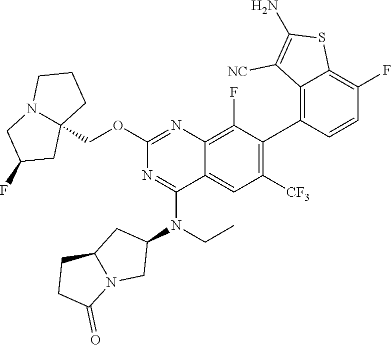 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@@H]1C[C@@H]2CCC(=O)N2C1] | LCMS m/z [M + 1]: 730.1 1H NMR (400 MHz, CD3OD) δ 8.20 (s, 1H), 7.24- 7.17 (m, 1H), 7.00 (t, 1H), 5.39 (s, 0.5H), 5.16 (s, 0.5H), 5.19-5.10 (m, 1H), 4.34-4.18 (m, 2H), 4.15- 4.06 (m, 1H), 3.93-3.70 (m, 3H), 3.55-3.47 (m, 1H), 3.42-3.32 (m, 1H), 3.26-3.21 (m, 1H), 3.09-3.00 (m, 1H), 2.82-2.72 (m, 1H), 2.57-2.48 (m, 1H), 2.45-2.34 (m, 2H), 2.30-2.15 (m, 3H), 2.05-1.88 (m, 4H), 1.49- 1.44 (m, 2H), 1.32-1.26 (m, 3H). |
| 511a | 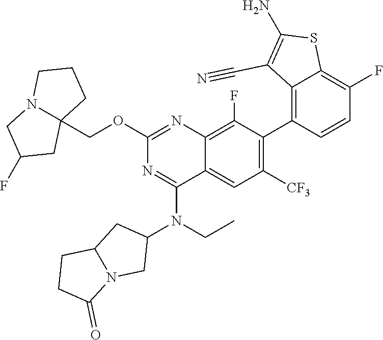 [SMILES: CCN(c1nc(OCC23CCCN2CC(F)C3)nc2c(F)c(-c3ccc(F)c4sc(N)c(C#N)c34)c(C(F)(F)F)cc12)C1CC2CCC(=O)N2C1] | |
| 512 | 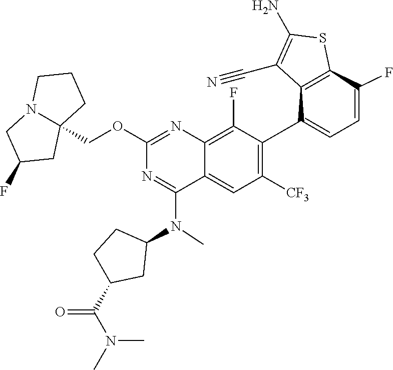 | LCMS m/z [M + 1]: 732.3 1H-NMR (400 MHz, CD3OD-d): 8.30 (s, 1 H), 7.21 (t, J = 8.0 Hz, 1 H), 7.01 (t, J = 9.2 Hz, 1 H), 5.28-5.19 (m, 2 H), 4.44 (t, J = 11.2 Hz, 1 H), 4.37 (d, J = 10.8 Hz, 1 H), 3.48-3.35 (m, 7 H), 3.15 (s, 3 H), 3.09-3.08 (m, 1 H), 2.98 (s, 3 H), 2.48-1.83 (m, 12H). |
| 513 | 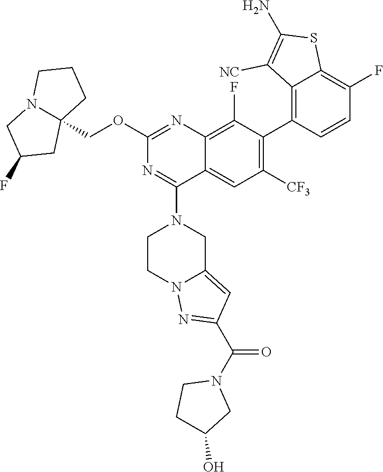 | LCMS m/z [M + 1]: 798.1 1H NMR (400 MHz, CD3OD) δ 8.23 (s, 1H), 7.22 (t, 1H), 7.01 (t, 1H), 6.65 (s, 1H), 5.36 (s, 0.5H), 5.24 (s, 2.5H), 4.33-4.53 (m, 6H), 4.26-4.29 (m, 1H), 3.97- 4.11 (m, 2H), 3.60-3.74 (m, 2H), 3.19-3.24 (m, 3H), 2.98-3.02 (m, 1H), 2.12-2.36 (m, 3H), 1.88-2.08 (m, 5H) |
| 513a | 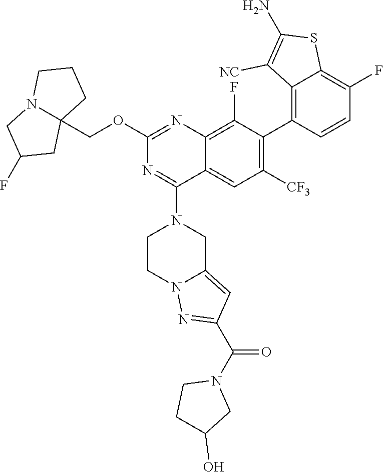 | |
| 514 | 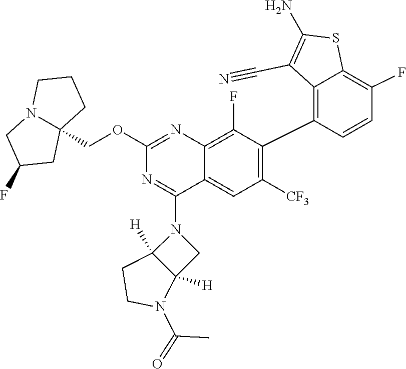 [SMILES: CC(=O)N1CC[C@@H]2[C@H]1CN2c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 702.3 1H NMR (400 MHz, CD3OD) δ 8.06 (s, 1H), 7.20 (t, 1H), 7.00 (t, 1H), 5.66 (s, 0.5H), 5.55 (s, 0.5H), 5.47 (s, 0.5H), 5.34 (s, 0.5H), 4.92 (t, 2H), 4.53-4.48 (m, 1H), 4.42-4.37 (m, 1H), 4.10-3.85 (m, 2H), 3.72-3.48 (m, 4H), 3.25-3.17 (m, 1H), 2.70-2.25 (m, 5H), 2.20- 1.90 (m, 6H). |
| 514a | 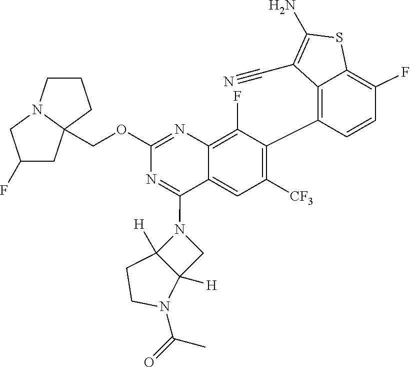 [SMILES: CC(=O)N1CCC2C1CN2c1nc(OCC23CCCN2CC(F)C3)nc2c(F)c(-c3ccc(F)c4sc(N)c(C#N)c34)c(C(F)(F)F)cc12] | |
| 515 | 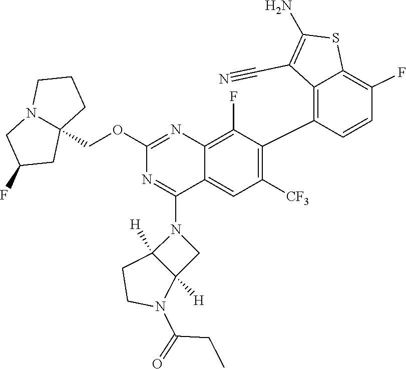 [SMILES: CCC(=O)N1CC[C@@H]2[C@H]1CN2c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 716.3 1H NMR (400 MHz, CD3OD) δ 8.06 (s, 1H), 7.19 (t, 1H), 7.00 (t, 1H), 5.66 (s, 0.5H), 5.52 (s, 0.5H), 5.44 (s, 0.5H), 5.31 (s, 0.5H), 4.93 (s, 2H), 4.46 (t, 1H), 4.37-4.32 (m, 1H), 4.05 (t, 1H), 3.90-3.85 (m, 1H), 3.75-3.37 (m, 4H), 3.21-3.13 (m, 1H), 2.67-2.44 (m, 3H), 2.39-1.94 (m, 7H), 1.20-1.07 (m, 3H). |
| 515a | 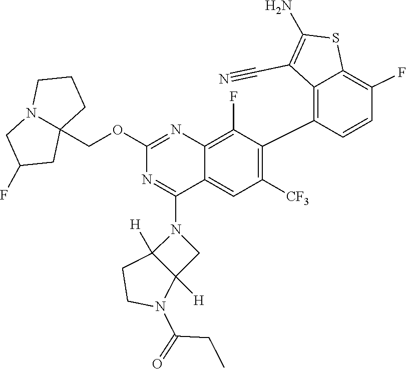 [SMILES: CCC(=O)N1CCC2C1CN2c1nc(OCC23CCCN2CC(F)C3)nc2c(F)c(-c3ccc(F)c4sc(N)c(C#N)c34)c(C(F)(F)F)cc12] | |
| 516 | 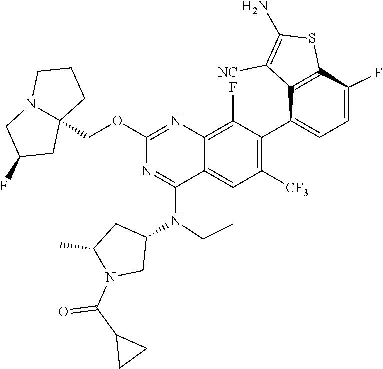 | LCMS m/z [M + 1]: 758.2 1H NMR (400 MHz, CD3OD) δ 8.257 (s, 1H), 7.216 (t, 1H), 7.011 (t, 1H), 5.392 (s, 0.5H), 5.257 (s, 0.5H), 4.839 (m, 1H), 4.394-4.248 (m, 3H), 4.090 (m, 1H), 3.911-3.855 (m, 3H), 3.126-3.061 (m, 2H), 2.772 (s, 1H), 2.572 (m, 1H), 2.293-2.182 (m, 4H), 2.158-2.030 (m, 5H), 1.470-1.371 (m, 6H), 0.896- 0.797 (m, 4H). |
| 517 | 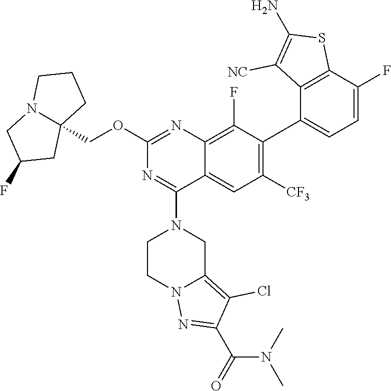 [SMILES: CN(C)C(=O)c1nn2c(c1Cl)CN(c1nc(OC[C@@]34CCCN3C[C@H](F)C4)nc3c(F)c(-c4ccc(F)c5sc(N)c(C#N)c45)c(C(F)(F)F)cc13)CC2] | LCMS m/z [M + 1]: 790.2 1H NMR (400 MHz, CD3OD) δ 8.17 (s, 1H), 7.23- 7.21 (m, 1H), 7.04-7.00 (m, 1H), 5.60 (s, 0.5H), 5.47 (s, 0.5H), 4.69-4.59 (m, 2H), 4.28-4.24 (m, 1H), 4.00-3.86 (m, 6H), 3.41 (s, 1H), 3.13 (s, 1H), 2.59- 2.16 (m, 8H), 1.81-1.68 (m, 6H). |
| 517a | 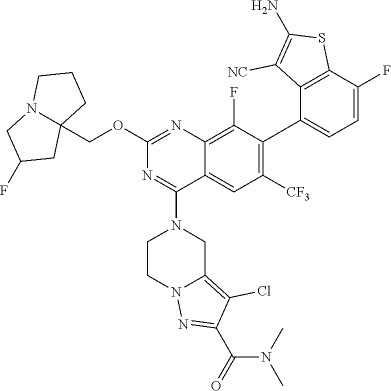 [SMILES: CN(C)C(=O)c1nn2c(c1Cl)CN(c1nc(OCC34CCCN3CC(F)C4)nc3c(F)c(-c4ccc(F)c5sc(N)c(C#N)c45)c(C(F)(F)F)cc13)CC2] | |
| 518 | 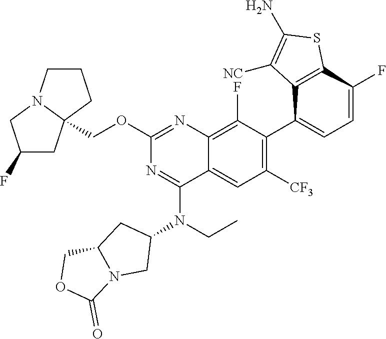 | LCMS m/z [M + 1]: 732.1 1H NMR (400 MHz, CD3OD) δ 8.18 (s, 1H), 7.19- 7.22 (m, 1H), 6.98-7.03 (m, 1H), 5.39 (s, 0.5H), 5.26 (s, 0.5H), 4.97-5.01 (m, 1H), 4.59-4.63 (m, 1H), 4.37-4.40 (m, 1H), 4.21-4.32 (m, 3H), 3.61-3.86 (m, 4H), 3.45 (s, 1H), 3.24 (s, 2H), 3.06 (m, 1H), 2.18- 2.45 (m, 5H), 1.91-2.02 (m, 3H), 1.46-1.50 (m, 3H). |
| 519 | 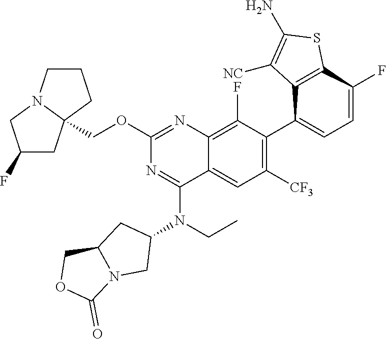 | LCMS m/z [M + 1]: 732.1 1H NMR (400 MHz, CD3OD) δ 8.19 (s, 1H), 7.20 (d, J = 4.8 Hz, 1H), 7.04-6.99 (m, 1H), 5.41 (s, 0.63H), 5.28 (s, 0.55H), 4.85-4.76 (m, 2H), 4.63-4.56 (m, 1H), 4.47-4.18 (m, 3H), 4.14-4.09 (m, 1H), 3.95-3.78 (m, 2H), 3.48-3.34 (m, 4H), 3.13(s, 1H), 2.52-1.94 (m, 8H), 1.53-1.49 (m, 3H). |
| 519a | 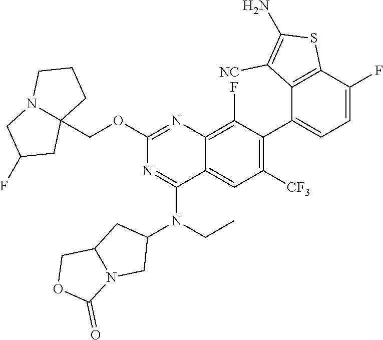 [SMILES: CCN(c1nc(OCC23CCCN2CC(F)C3)nc2c(F)c(-c3ccc(F)c4sc(N)c(C#N)c34)c(C(F)(F)F)cc12)C1CC2COC(=O)N2C1] | |
| 520 | 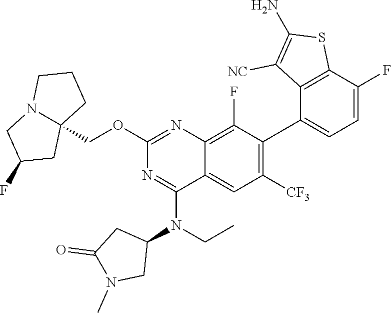 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@@H]1CC(=O)N(C)C1] | LCMS m/z [M + 1]: 704.3 1H NMR (400 MHz, CD3OD) δ 8.17 (s, 1H), 7.28- 7.18 (m, 1H), 7.01 (m, 1H), 5.49 (s, 0.5H), 5.35 (s, 0.5H), 4.70 (s, 1H), 4.34-4.18 (m, 2H), 3.96-3.72 (m, 4H), 3.69-3.38 (m, 3H), 3.18 (s, 1H), 2.95 (m, 4H), 2.89-2.79 (m, 1H), 2.53-2.18 (m, 3H), 2.06 (m, 3H), 1.55 (m, 3H). |
| 521 | 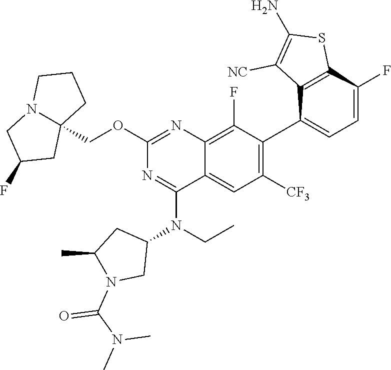 | LCMS m/z [M + 1]: 761.1 1H NMR (400 MHz, CD3OD) δ 8.22 (s, 1H), 7.23- 7.19 (m, 1H), 7.01 (t, J = 8.8 Hz, 1H), 5.40 (d, J = 56 Hz, 1H), 5.16 (s, 1H), 4.49-4.35 (m, 2H), 4.05-4.00 (m, 1H), 3.84-3.71 (m, 2H), 3.58-3.48 (m, 4H), 3.23- 3.17 (m, 2H), 2.88 (s, 6H), 2.60-1.96 (m, 8H), 1.37- 1.36 (m, 3H), 1.21 (d, J = 6.4 Hz, 3H). |
| 521a | 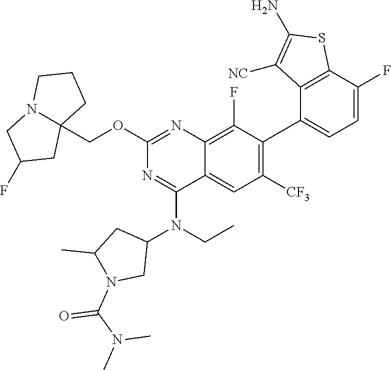 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)N(C)C)C1] | |
| 522 | 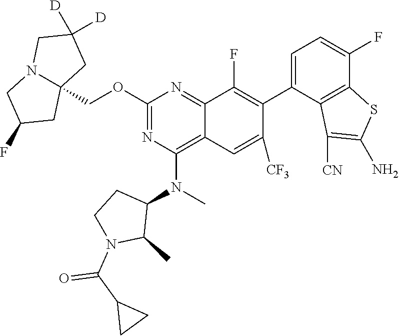 [SMILES: [2H]C1([2H])CN2C[C@H](F)C[C@]2(COc2nc(N(C)[C@@H]3CCN(C(=O)C4CC4)[C@@H]3C)c3cc(C(F)(F)F)c(-c4ccc(F)c5sc(N)c(C#N)c45)c(F)c3n2)C1] | LCMS m/z [M + 1]: 746.3 1H NMR (400 MHz, CD3OD): δ 8.41-8.44 (m, 1H), 7.22-7.25 (m, 1H), 7.03 (t, J = 8.88 Hz, 1H), 5.55 (d, J = 52.74 Hz, 1H), 4.93-5.00 (m, 1H), 4.59-4.83 (m, 3H), 3.66-4.06 (m, 8H), 3.43-3.50 (m, 1H), 2.29-2.77 (m, 6H), 1.81-1.87 (m, 1H), 0.83-1.26 (m, 7H). |
| 522a | 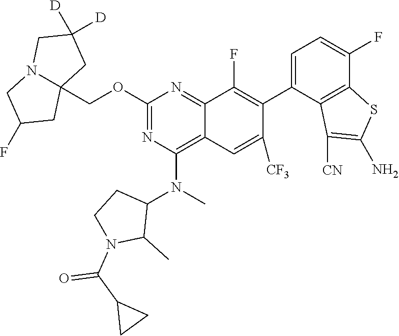 [SMILES: [2H]C1([2H])CN2CC(F)CC2(COc2nc(N(C)C3CCN(C(=O)C4CC4)C3C)c3cc(C(F)(F)F)c(-c4ccc(F)c5sc(N)c(C#N)c45)c(F)c3n2)C1] | |
| 523 | 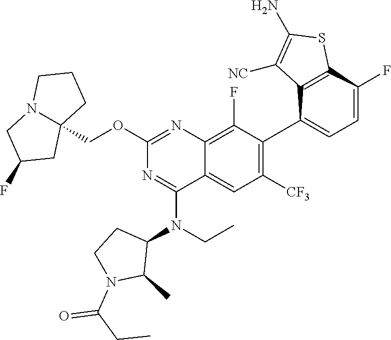 | LCMS m/z [M + 1]: 746.1 1H NMR (400 MHz, CD3OD) δ 8.32 (s, 1H), 7.22 (s, 1H), 7.04-6.97 (m, 1H), 5.48 (s, 0.5H), 5.30 (s, 0.5H), 5.04-4.91 (m, 1H), 4.76-4.66 (m, 1H), 4.47- 4.31 (m, 2H), 4.07-3.76 (m, 3H), 3.58-3.35 (m, 3H), 3.25-3.05 (m, 2H), 2.56-1.96 (m, 10H), 1.42-1.33 (m, 3H), 1.18-0.94 (m, 6H). |
| 524 | 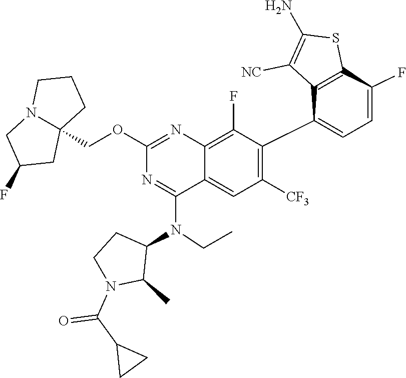 | LCMS m/z [M + 1]: 758.2 1H NMR (400 MHz, CD3OD) δ 8.39-8.29 (m, 1H), 7.22 (s, 1H), 7.01 (t, 1H), 5.45-5.41 (m, 0.5H), 5.32- 5.28 (m, 0.5H), 5.13-5.08 (m, 0.5H), 5.01-4.97 (m, 0.5H), 4.79-4.68 (m, 1H), 4.47-4.30 (m, 2H), 4.11- 3.68 (m, 4H), 3.57-3.34 (m, 3H), 3.17-3.08 (m, 1H), 2.68-2.30 (m, 4H), 2.19-1.82 (m, 5H), 1.62-1.21 (m, 6H), 1.16-0.89 (m, 4H) |
| 525 | 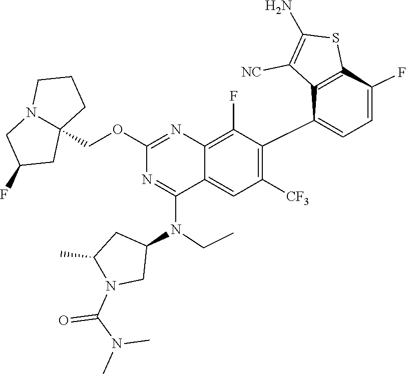 | LCMS m/z [M + 1]: 761.27 1H NMR (400 MHz, DMSO) δ 8.23 (s, 1H), 7.28- 7.22 (m, 1H), 7.08-7.01 (m, 1H), 5.41 (s, 0.5H), 5.28 (s, 0.5H), 5.16 (s, 1H), 4.53-4.44 (m, 1H), 4.38-4.27 (m, 2H), 4.07-3.85 (m, 3H), 3.75-3.66 (m, 1H), 3.61- 3.65 (m,1H), 3.28-3.23 (m, 1H), 3.10-3.03 (m, 1H), 2.90 (s, 6H), 2.66-2.58 (m, 1H), 2.46-2.36 (m, 1H), 2.34-2.27 (m, 1H), 2.21-2.11 (m,2H), 2.09-1.84 (m, 4H), 1.44-1.34 (m, 3H), 1.26-1.21 (m, 3H). |
| 526 | 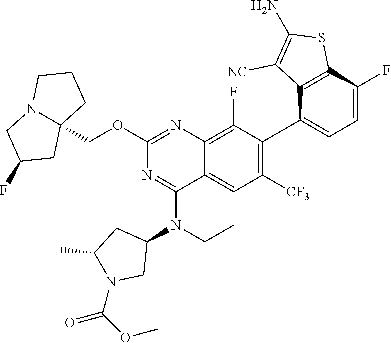 | LCMS m/z [M + 1]: 748.24 1H NMR (400 MHz, DMSO) δ 8.25 (s, 1H), 7.29- 7.22 (m, 1H), 7.08-7.00 (m, 1H), 5.41 (s, 0.5H), 5.27 (s, 0.5H), 5.24-5.15 (m, 1H), 4.37-4.32 (m, 1H), 4.29-4.19 (m, 2H), 3.98-3.71 (m, 6H), 3.68-3.62 (m, 1H), 3.29-3.23 (m, 2H), 3.09-3.01 (m, 1H), 2.73-2.63 (m, 1H), 2.43-2.10 (m, 5H), 2.08-1.98 (m, 2H), 1.97- 1.88 (m, 1H), 1.49-1.41 (m, 3H), 1.33-1.30 (m, 3H). |
| 526a | 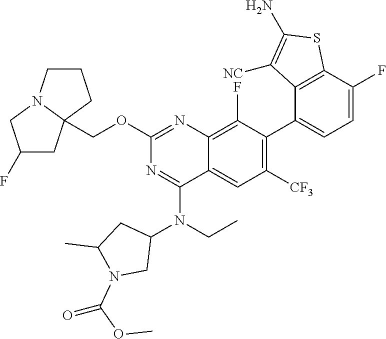 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)OC)C1] | |
| 527 | 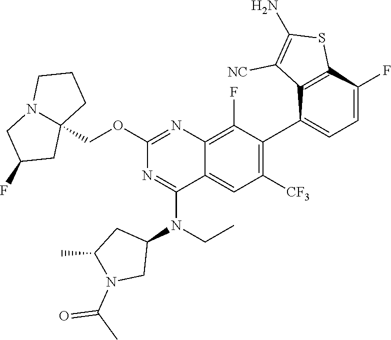 | LCMS m/z [M + 1]: 732.25 1H NMR (400 MHz, DMSO) δ 8.26 (s, 1H), 7.29- 7.22 (m, 1H), 7.09-7.00 (m, 1H), 5.41 (s, 0.5H), 5.30- 5.18 (m, 1.5H), 4.49-4.31 (m, 2H), 4.29-4.24 (m, 1H), 4.05-3.86 (m, 2H), 3.84-3.67 (m, 2H), 3.29-3.23 (m, 2H), 3.11-3.01 (m, 1H), 2.78-2.67 (m, 1H), 2.48- 1.81 (m, 11H), 1.49-1.29 (m, 6H). |
| 528 | 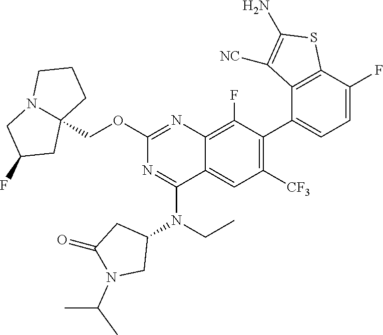 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@H]1CC(=O)N(C(C)C)C1] | LCMS m/z [M + 1]: 732.1 1H NMR (400 MHz, CD3OD) δ 8.190 (s, 1H), 7.214-7.200 (s, 1H), 7.018 (t, 1H), 5.486 (s, 0.5H), 5.349 (s, 0.5H), 4.426-4.340 (m, 3H), 3.934-3.864 (m, 3H), 3.717-3.688 (m, 2H), 3.570 (s, 2H), 3.028- 2.959 (m, 1H), 2.869-2.801 (m, 2H) , 2.508-2.219 (m, 4H), 2.153 (m, 2H), 2.031 (m, 2H), 1.543-1.532 (m, 3H), 1.218-1.201 (m, 6H). |
| 528a | 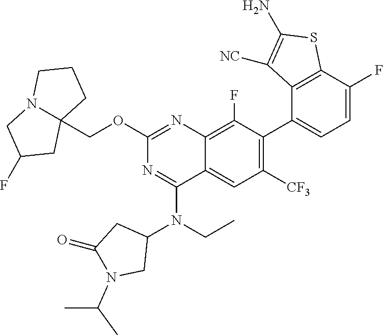 [SMILES: CCN(c1nc(OCC23CCCN2CC(F)C3)nc2c(F)c(-c3ccc(F)c4sc(N)c(C#N)c34)c(C(F)(F)F)cc12)C1CC(=O)N(C(C)C)C1] | |
| 529 | 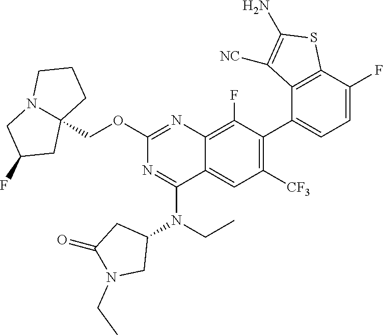 [SMILES: CCN1C[C@@H](N(CC)c2nc(OC[C@@]34CCCN3C[C@H](F)C4)nc3c(F)c(-c4ccc(F)c5sc(N)c(C#N)c45)c(C(F)(F)F)cc23)CC1=O] | LCMS m/z [M + 1]: 718.1 1H NMR (400 MHz, CD3OD) δ 8.181 (s, 1H), 7.233-7.200 (s, 1H), 7.017 (t, 1H), 5.486 (s, 0.5H), 5.378 (s, 0.5H), 4.731 (m, 1H), 4.393-4.298 (m, 2H), 4.07 (m, 1H), 3.968-3.836 (m, 3H), 3.778-3.713 (m, 1H), 3.631-3.478 (m, 3H), 3.223 (m, 1H), 3.016- 2.958 (m, 1H), 2.886-2.815 (m, 1H), 2.508-2.251 (m, 4H), 3.142-1.932 (m, 4H), 1.546 (t, 3H), 1.200 (t, 3H). |
| 529a | 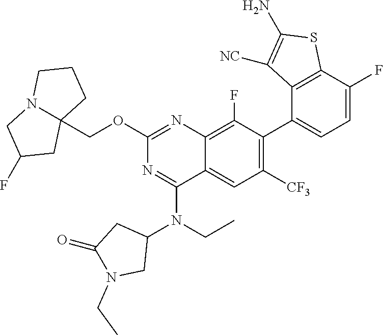 [SMILES: CCN1CC(N(CC)c2nc(OCC34CCCN3CC(F)C4)nc3c(F)c(-c4ccc(F)c5sc(N)c(C#N)c45)c(C(F)(F)F)cc23)CC1=O] | |
| 530 | 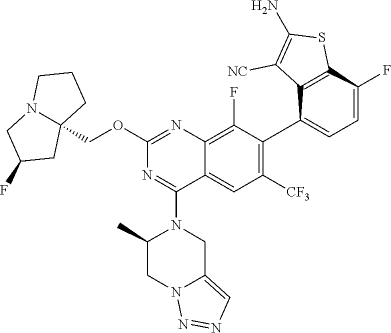 | LCMS m/z [M + 1]: 700.20 1H-NMR (400 MHz, CD3OD-d): 8.51 (brs, 1 H), 8.29 (s, 1 H), 7.68 (s, 1 H), 7.23 (s, 1 H), 7.02 (t, J = 8.8 Hz, 1 H), 5.43-5.39 (m, 3 H), 5.24 (s, 1 H), 5.00 (s, 1 H), 4.58 (d, J = 12.4 Hz, 1 H), 4.47 (d, J = 11.2 Hz, 1 H), 4.36 (d, J = 10.8 Hz, 1 H), 4.14-4.09 (m, 1 H), 3.52-3.39 (m, 3 H), 3.13 (s, 1 H), 2.45-2.21 (m, 3 H), 2.09-1.96 (m, 3 H), 1.80 (d, J = 6.0 Hz, 1 H). |
| 531a | 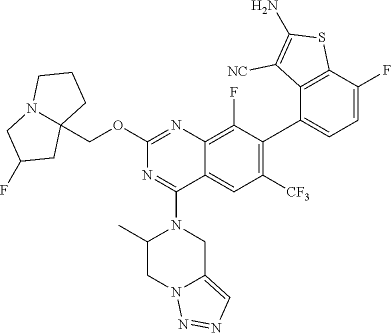 [SMILES: CC1Cn2nncc2CN1c1nc(OCC23CCCN2CC(F)C3)nc2c(F)c(-c3ccc(F)c4sc(N)c(C#N)c34)c(C(F)(F)F)cc12] | |
| 531 | 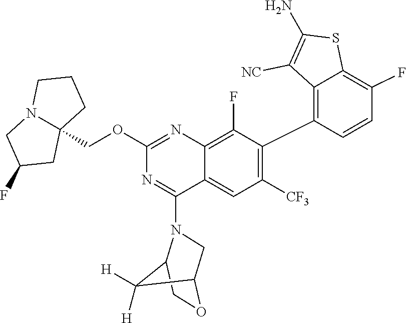 [SMILES: N#Cc1c(N)sc2c(F)ccc(-c3c(C(F)(F)F)cc4c(N5CC6CC5CO6)nc(OC[C@@]56CCCN5C[C@H](F)C6)nc4c3F)c12] | LCMS m/z [M + 1]: 679.0 1H NMR (400 MHz, CD3OD) δ 8.26 (s, 1H), 7.20 (s, 1H), 7.03-6.99 (m, 1H), 5.51 (s, 0.5H), 5.41 (s, 0.45H), 5.37 (s, 1H), 5.34 (s, 0.39 H), 5.28 (s, 0.47H), 4.63-4.55 (m, 1H), 4.43-4.26 (m, 4H), 4.10 (d, J = 10 Hz, 1H), 3.58-3.32 (m, 3H), 3.13 (s, 1H), 2.40-1.93 (m, 6H). |
| 531a | 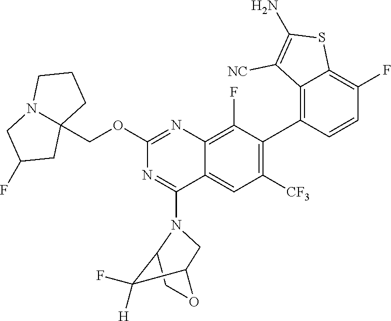 [SMILES: N#Cc1c(N)sc2c(F)ccc(-c3c(C(F)(F)F)cc4c(N5CC6OCC5C6F)nc(OCC56CCCN5CC(F)C6)nc4c3F)c12] | |
| 532 | 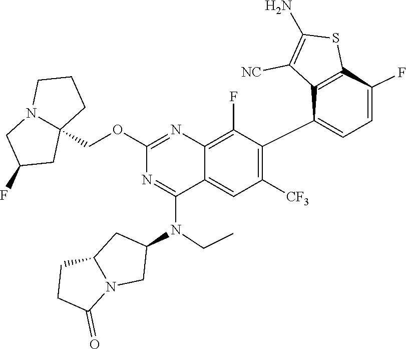 | LCMS m/z [M + 1]: 730.1 1H NMR (400 MHz, CD3OD) δ 8.19 (s, 1H), 7.23- 7.20 (m, 1H), 7.03-6.99 (m, 1H), 5.40 (s, 0.57H), 5.26 (s, 0.65H), 4.91 (s, 1H), 4.67 (d, J = 8 Hz, 1H), 4.37-4.31 (m, 1H), 4.14-4.09 (m, 2H), 3.75-3.35 (s, 2H), 3.13-3.08 (m, 1H), 2.77-2.67 (m, 1H), 2.51-1.79 (m, 13H), 1.50-1.47 (m, 3H). |
| 533 | 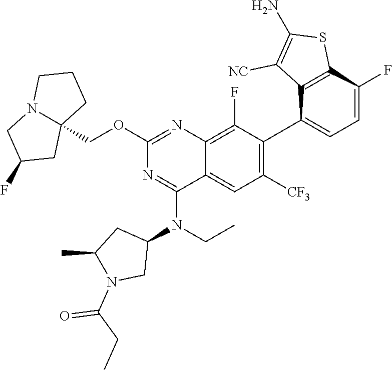 | LCMS m/z [M + 1]: 746.1 1H NMR (400 MHz, CD3OD) δ 8.23 (s, 1H), 7.23- 7.10 (m, 1H), 7.03-6.99 (m, 1H), 5.37 (s, 0.63H), 5.24 (s, 0.58H), 4.98-4.86 (m, 1H), 4.30-4.25 (m, 2 H), 4.14-4.05 (m, 2H), 3.89-3.83 (m, 2H), 3.73-3.68 (m, 1H), 3.27-3.18 (m, 2H), 3.03 (s, 1H), 2.65-2.62 (m, 1H), 2.38-1.89 (m, 10H), 1.47-1.42 (m, 6H), 1.18-1.11 (m, 3H). |
| 534 | 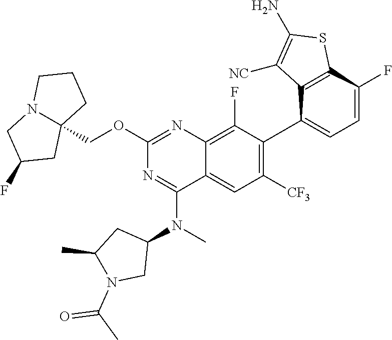 | LCMS m/z [M + 1]: 732.1 1H NMR (400 MHz, CD3OD) δ 8.23 (s, 1H), 7.21 (s, 1H), 7.03-6.99 (m, 1H), 5.38 (s, 0.65H), 5.24 (s, 0.59H), 4.83-4.73 (m, 1H), 4.34-4.29 (m, 2 H), 4.11- 4.03 (m, 2H), 3.89-3.73 (m, 3H), 3.15-3.26 (m, 2H), 3.04 (s, 1H), 2.63 (s, 1H), 2.34-1.90 (m, 11H), 1.48- 1.29 (m, 6H). |
| 535 | 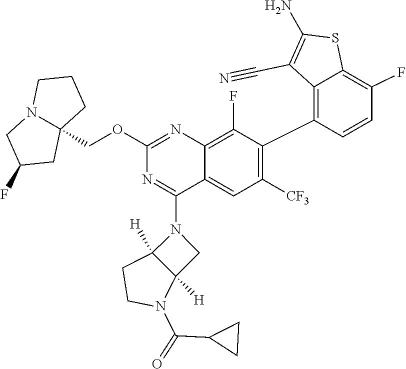 [SMILES: N#Cc1c(N)sc2c(F)ccc(-c3c(C(F)(F)F)cc4c(N5C[C@@H]6[C@H]5CCN6C(=O)C5CC5)nc(OC[C@@]56CCCN5C[C@H](F)C6)nc4c3F)c12] | LCMS m/z [M + 1]: 728.3 1H NMR (400 MHz, CD3OD) δ 8.07 (d, 1H), 7.19 (t, 1H), 7.00 (t, 1H), 5.68 (s, 0.5H), 5.55 (s, 0.5H), 5.47 (d, 1H), 5.32 (s, 0.5H), 5.17 (s, 0.5H), 4.93 (s, 1H), 4.47 (t, 1H), 4.38-4.17 (m, 2H), 4.02 (t, 1H), 3.80- 3.35 (m, 4H), 3.22-3.13 (m, 1H), 2.75-2.45 (m, 2H), 2.40-2.07 (m, 5H), 2.03-1.80 (m, 2H), 0.94-0.75 (m, 4H). |
| 535a | 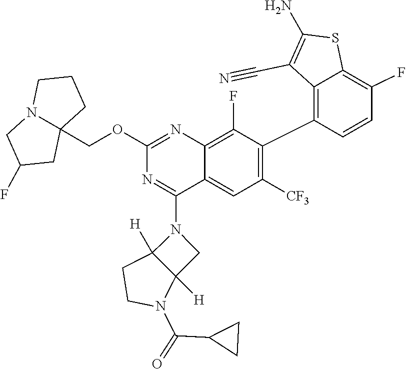 [SMILES: N#Cc1c(N)sc2c(F)ccc(-c3c(C(F)(F)F)cc4c(N5CC6C5CCN6C(=O)C5CC5)nc(OCC56CCCN5CC(F)C6)nc4c3F)c12] | |
| 536 | 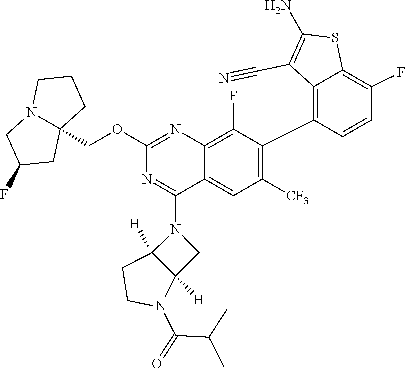 [SMILES: CC(C)C(=O)N1CC[C@@H]2[C@H]1CN2c1nc(OC[C@@]23CCCN2C[C@H](F)C3)nc2c(F)c(-c3ccc(F)c4sc(N)c(C#N)c34)c(C(F)(F)F)cc12] | LCMS m/z [M + 1]: 730.2 1H NMR (400 MHz, CD3OD) δ 8.06 (s, 1H), 7.19 (t, 1H), 7.00 (t, 1H), 5.67 (s, 0.5H), 5.53 (s, 0.5H), 5.44 (s, 0.5H), 5.30 (s, 0.5H), 5.06-4.90 (m, 2H), 4.45 (t, 1H), 4.36-4.27 (m, 1H), 4.25-4.10 (m, 1H), 4.00-3.85 (m, 1H), 3.60-3.36 (m, 3H), 3.21-3.12 (m, 1H), 2.94- 2.54 (m, 3H), 2.50-2.18 (m, 4H), 2.14-1.90 (m, 3H), 1.20-1.04 (m, 6H). |
| 536a | 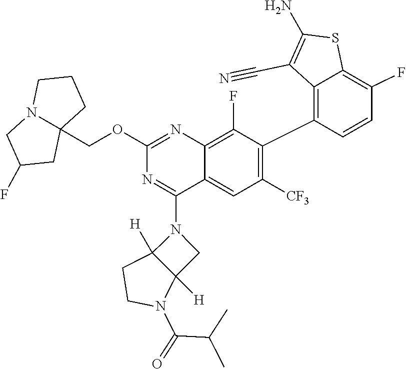 [SMILES: CC(C)C(=O)N1CCC2C1CN2c1nc(OCC23CCCN2CC(F)C3)nc2c(F)c(-c3ccc(F)c4sc(N)c(C#N)c34)c(C(F)(F)F)cc12] | |
| 537 | 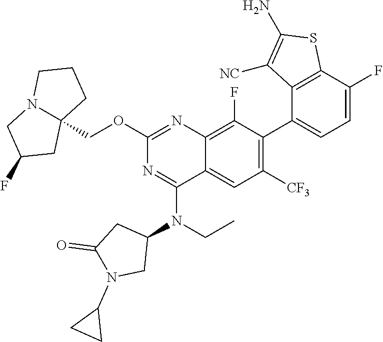 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@@H]1CC(=O)N(C2CC2)C1] | LCMS m/z [M + 1]: 730.0 1HNMR (400 MHz, CD3OD) δ 8.41 (s, 1H), 8.19 (s, 1H), 7.22-7.20 (m, 1H), 7.05-7.00 (m, 1H) 5.62-5.49 (m, 1H), 4.57-4.49 (m, 3H), 3.84-3.63 (m, 7H), 3.40 (m, 1H), 2.95-1.92 (m, 9H), 1.564 (m, 3H), 0.86-0.73 (m, 4H). |
| 538 | 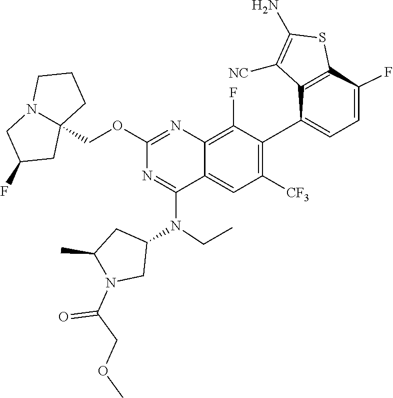 | LCMS m/z [M + 1]: 762.3 1H NMR (400 MHz, CD3OD) δ 8.23 (s, 1H), 7.23- 7.20 (m, 1H), 7.011 (t, J = 5.2 Hz, 1H), 5.40-5.20 (m, 2H), 4.49-4.45 (m, 1H), 4.33-4.23 (m, 2H), 4.16-4.08 (m, 2H), 4.06-3.70 (m, 4H), 3.44-3.42 (m, 4H), 3.24- 3.20 (m, 1H), 3.10-3.04 (M, 1H), 2.67-2.59 (m, 1H), 2.36-1.92 (m, 8H), 1.46-1.45 (m, 3H), 1.30-1.25 (m, 3H). |
| 538a | 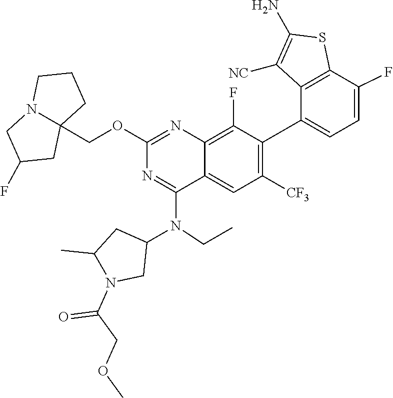 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)COC)C1] | |
| 539 | 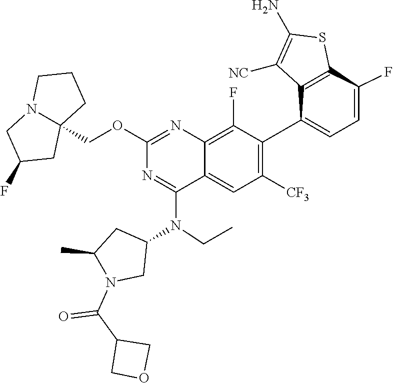 | LCMS m/z [M + 1]: 774.2 1H NMR (400 MHz, DMSO-d6) δ8.23 (s, 1H), 7.20 (s, 1H), 7.01 (s, 1H), 5.43-5.20 (m, 2H), 4.99-4.92 (m, 2H), 4.41-4.23 (m, 2H), 4.21-4.09 (m, 1H), 3.95- 3.70 (m, 3H), 3.61-3.50 (m, 1H), 3.50-3.38 (m, 4H), 3.13 (s, 1H), 2.64 (s, 1H), 2.50-1.82 (m, 8H), 1.43 (s, 3H), 1.29 (s, 3H). |
| 539a | 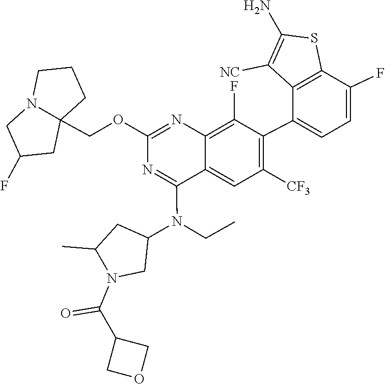 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)C2COC2)C1] | |
| 540 | 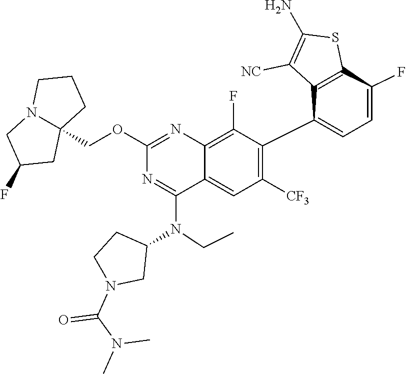 | LCMS m/z [M + 1]: 747.1 1H NMR (400 MHz, CD3OD) δ 8.28 (s, 1H), 7.21- 7.25 (m, 1H), 7.02 (t, 1H), 6.64 (s, 2H), 5.58 (s, 0.5H), 5.45 (s, 0.5H), 5.01-5.05 (m, 1H), 4.55-4.67 (m, 2H), 3.71-3.96 (m, 6H), 3.53-3.65 (m, 3H), 3.34- 3.41 (m, 1H), 2.89 (s, 6H), 2.27-2.67 (m, 7H), 2.09- 2.15 (m, 1H), 1.47 (t, 3H). |
| 541 | 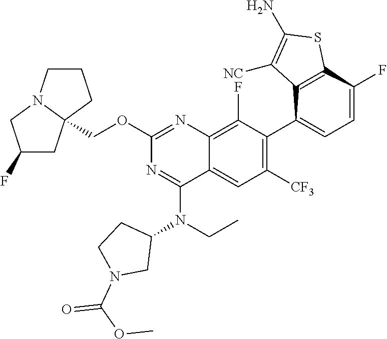 | LCMS m/z [M + 1]: 734.1 1H NMR (400 MHz, CD3OD) δ 8.26 (s, 1H), 7.23 (m, 1H), 7.02 (t, 1H), 6.58-6.64 (m, 2H), 5.58 (s, 0.5H), 5.45 (s, 0.5H), 5.02-5.04 (m, 1H), 4.54-4.61 (m, 2H), 3.72-3.90 (m, 9H), 3.37-3.63 (m, 3H), 3.21- 3.25 (m, 1H), 2.27-2.67 (m, 7H), 2.09 (s, 1H), 1.46- 1.47 (m, 3H). |
| 542 | 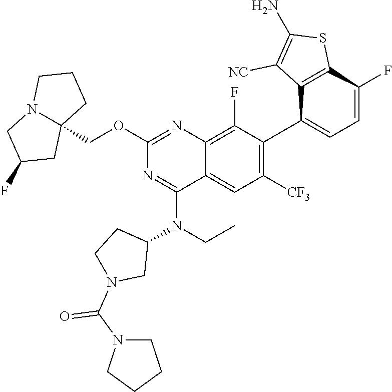 | LCMS m/z [M + 1]: 774.3 1H NMR (400 MHz, CD3OD) δ 8.28 (s, 1H), 7.20- 7.23 (m, 1H), 6.99-7.03 (m, 1H), 5.44 (s, 0.5H), 5.31 (s, 0.5H), 4.99-5.03 (m, 1H), 4.33-4.44 (m, 2H), 3.86-3.92 (m, 3H), 3.40-3.69 (m, 10H), 3.14 (s, 1H), 2.31-2.44 (m, 4H), 2.09-2.21 (m, 3H), 1.83-1.91 (m, 5H), 1.44-1.48 (m, 3H). |
| 542a | 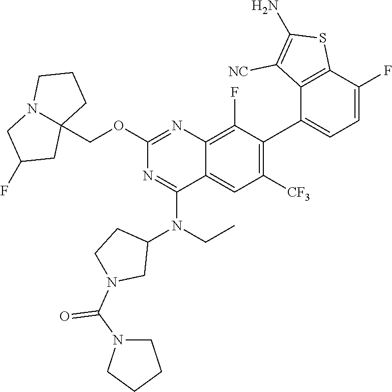 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2CCCC2)C1] | |
| 543 | 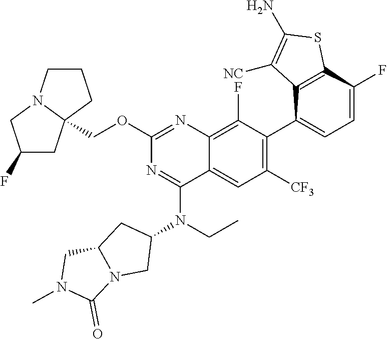 | LCMS m/z [M + 1]: 745.2 1H NMR (400 MHz, CD3OD) δ 8.19 (s, 1H), 7.20- 7.22 (m, 1H), 6.98-7.02 (m, 1H), 5.36 (s, 0.5H), 5.22 (s, 1.5H), 4.20-4.31 (m, 2H), 3.88 (m, 1H), 3.45-3.76 (m, 6H), 3.18-3.22 (m, 3H), 2.99 (s, 1H), 2.86 (s, 3H), 1.88-2.35 (m, 8H), 1.37 (s, 3H). |
| 543a | 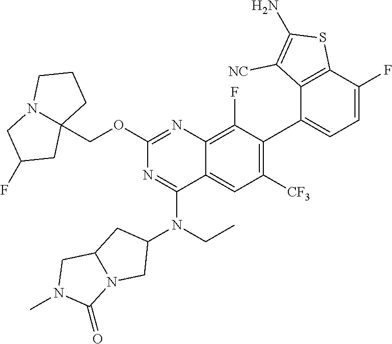 [SMILES: CCN(c1nc(OCC23CCCN2CC(F)C3)nc2c(F)c(-c3ccc(F)c4sc(N)c(C#N)c34)c(C(F)(F)F)cc12)C1CC2CN(C)C(=O)N2C1] | |
| 544 | 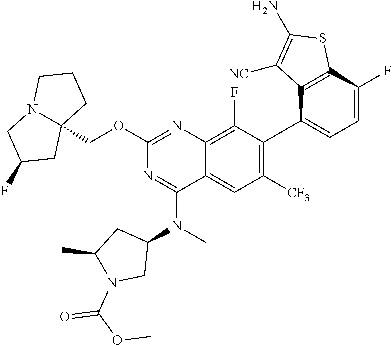 | LCMS m/z [M + 1]: 748.2 1H NMR (400 MHz, CD3OD) δ 8.24 (s, 1H), 7.23- 7.20 (m, 1H), 7.03-6.99 (m, 1H), 5.40 (s, 0.68H), 5.26 (s, 0.63H), 4.91 (s, 1H), 4.45-4.25 (m, 2H), 4.12 (s, 1H), 4.03-3.82 (m, 3H), 3.71 (s, 3H), 3.50 (s, 1H), 3.43-3.33 (m, 2H), 3.06 (s, 1H), 2.60 (s, 1H), 2.38-1.92 (m, 8H), 1.43 (d, J = 5.6 Hz, 6H). |
| 545 | 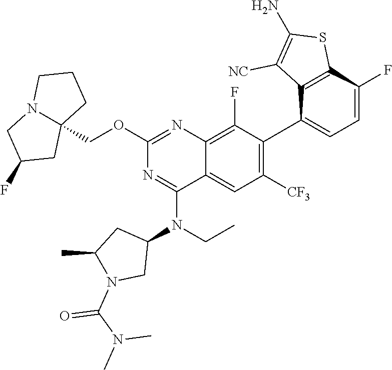 | LCMS m/z [M + 1]: 761.3 1H NMR (400 MHz, CD3OD) δ 8.24 (s, 1H), 7.21 (s, 1H), 7.03-6.99 (m, 1H), 5.37 (s, 0.61H), 5.24 (s, 0.60H), 4.68 (s, 1H), 4.28 (d, J = 13.2 Hz, 2H), 4.10 (s, 1H), 3.93-3.65 (m, 5H), 3.25 (s, 1H), 3.02 (s, 1H), 2.88 (s, 6H), 2.48 (s, 1H), 2.20-1.89 (m, 8H), 1.44 (d, J = 6.4 Hz, 3H), 1.30 (d, J = 5.6 Hz, 3H). |
| 546 | 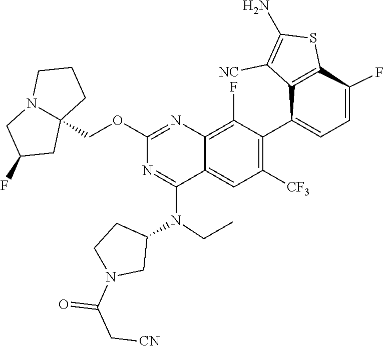 | LCMS m/z [M + 1]: 743.2 1H NMR (400 MHz, CD3OD) δ 8.48 (brs, 0.65H), 8.26 (s, 1H), 7.27-7.16 (m, 1H), 7.02 (t, J = 9.0 Hz, 1H), 5.51-5.35 (m, 1H), 5.07-4.96 (m, 1H), 4.55-4.33 (m, 2H), 4.11-3.38 (m, 9H), 3.29-3.21 (m, 3H), 2.64- 1.92 (m, 8H), 1.56-1.34 (m, 3H). |
| 546a | 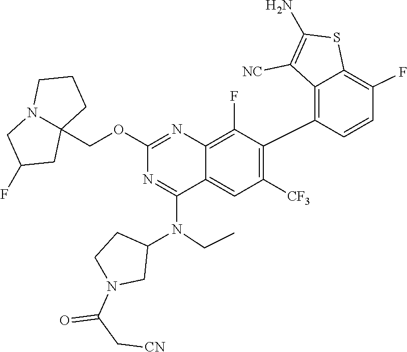 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)CC#N)C1] | |
| 547 | 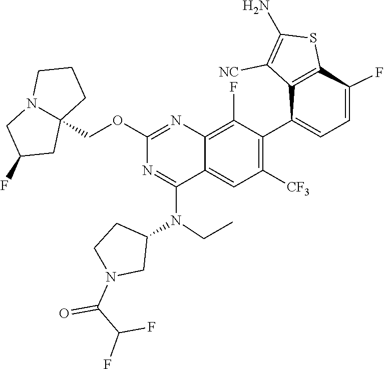 | LCMS m/z [M + 1]: 754.1 1H NMR (400 MHz, CD3OD) δ 8.24 (s, 1H), 7.20- 7.23 (m, 1H), 7.01 (t, 1H), 8.21-8.53 (m, 1H), 5.38 (s, 0.5H), 5.25 (s, 0.5H), 4.96-5.04 (m, 1H), 4.22- 4.33 (m, 2H), 3.71-4.07 (m, 5H), 3.30-3.57 (m, 2H), 3.25-3.34 (m, 2H), 3.04-3.05 (m, 1H), 2.25-2.58 (m, 5H), 2.01-2.13 (m, 2H), 1.90-1.91 (m, 1H), 1.44- 1.50 (m, 3H). |
| 547a | 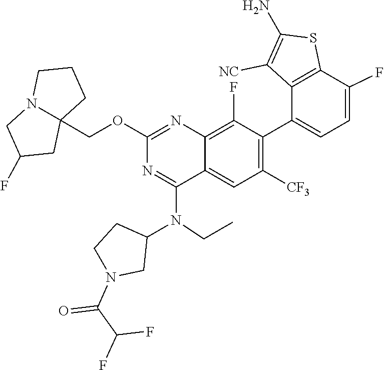 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C(F)F)C1] | |
| 548 | 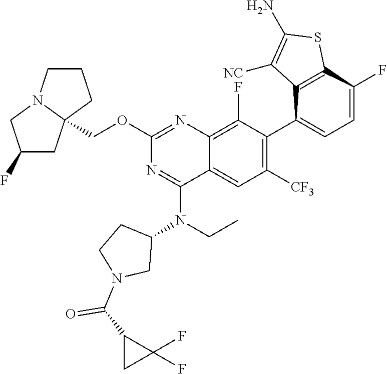 | LCMS m/z [M + 1]: 780.3 1H NMR (400 MHz, CD3OD) δ 8.25 (m, 1H), 7.26- 7.18 (m, 1H), 7.01 (m, 1H), 5.39 (s, 0.5H), 5.25 (s, 0.5H), 5.17-4.97 (m, 1H), 4.28 (m, 2H), 4.15-4.04 (m, 1H), 3.99-3.77 (m, 3H), 3.67 (m, 1H), 3.54-3.34 (m, 1H), 3.28-3.20 (m, 2H), 3.05 (m, 1H), 2.94-2.82 (m, 1H), 2.67-1.73 (m, 11H), 1.48 (m, 3H). |
| 548a | 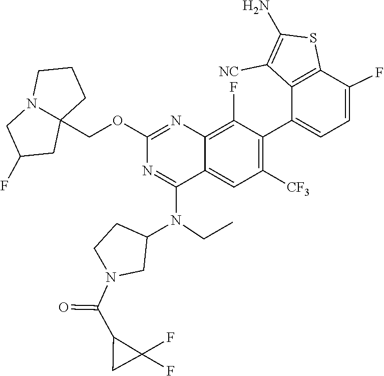 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CC2(F)F)C1] | |
| 549 | 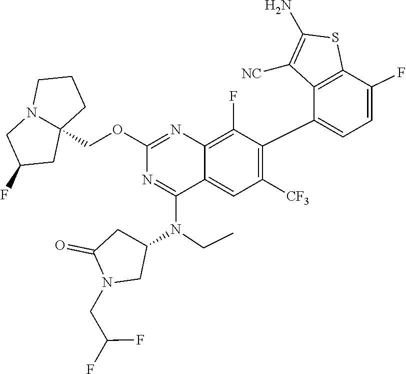 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@H]1CC(=O)N(CC(F)F)C1] | LCMS m/z [M + 1]: 754.2 1H NMR (400 MHz, CD3OD) δ 8.187 (s, 1H), 7.218-7.201 (m, 1H), 7.018 (t, 1H), 6.253 (s, 0.3H), 6.115 (s, 0.6H), 5.976 (s, 0.3H), 5.496 (s, 0.5H), 5.356 (s, 0.5H), 4.756 (s, 1H), 4.392-4.365 (m, 2H), 4.042 (m, 1H), 3.890-3.821 (m, 3H), 3.672 (d, 1H), 3.553 (m, 2H), 3.216 (s, 1H), 2.951-2.893 (m, 2H), 2.372-2.139 (m, 7H), 1.997 (s, 1H), 1.547 (t, 3H). |
| 549a | 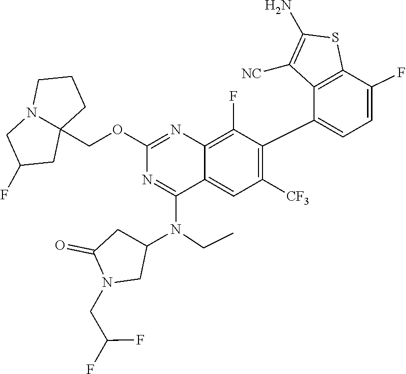 [SMILES: CCN(c1nc(OCC23CCCN2CC(F)C3)nc2c(F)c(-c3ccc(F)c4sc(N)c(C#N)c34)c(C(F)(F)F)cc12)C1CC(=O)N(CC(F)F)C1] | |
| 550 | 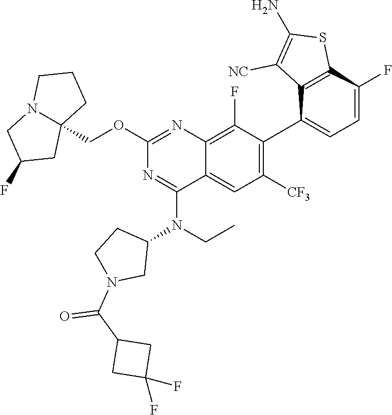 | LCMS m/z [M + 1]: 794.3 1H NMR (400 MHz, CD3OD) δ 8.242 (s, 1H), 7.205 (t, 1H), 7.011 (t, 1H), 5.376 (s, 0.5H), 5.241 (s, 0.5H), 5.055-4.985 (m, 1H), 4.326-4.205 (m, 2H), 4.022 (m, 1H), 3.850 (d, 2H), 3.684-3.601 (m, 1H), 3.478-3.447 (m, 1H) , 3.230-3.216 (m, 2H), 3.040 (m, 1H), 2.851-2.814 (m, 3H), 2.507-2.143 (m, 5H), 2.005 (m, 2H), 1.930-1.886 (m, 1H), 1.458-1.442 (m, 3H), 1.348-1.285 (m, 2H), 0.963-0.915 (m, 2H). |
| 550a | 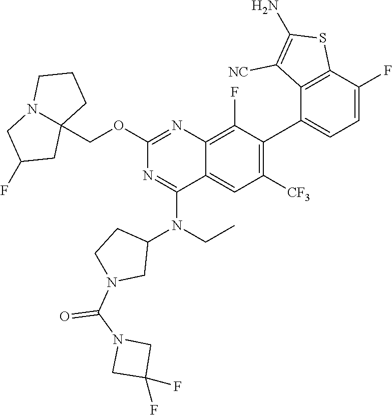 | |
| 551 | 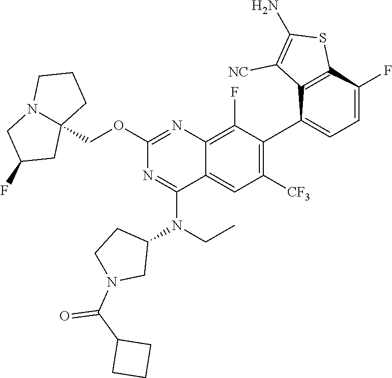 | LCMS m/z [M + 1]: 758.2 1H NMR (400 MHz, CD3OD) δ 8.23 (s, 1H), 7.19- 7.23 (m, 1H), 7.01 (t, 1H), 5.38 (s, 0.5H), 5.25 (s, 0.5H), 4.96-5.01 (m, 1H), 4.21-4.33 (m, 2H), 3.97 (t, 1H), 3.76-3.87 (m, 3H), 3.61 (t, 1H), 3.35-3.52 (m, 3H), 3.28-3.30 (m, 2H), 3.04 (s, 1H), 2.13-2.50 (m, 9H), 2.00-2.07 (m, 3H), 1.87-1.89 (m, 2H), 1.42-1.47 (m, 3H). |
| 551a | 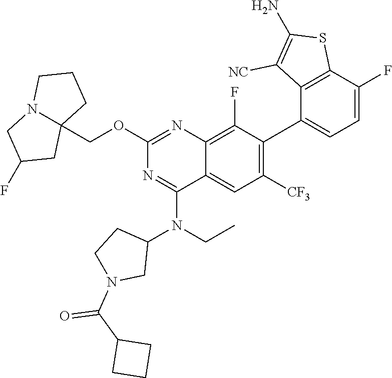 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CCC2)C1] | |
| 552 | 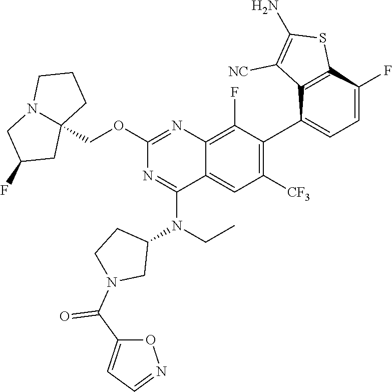 | LCMS m/z [M + 1]: 771.1 1HNMR (400 MHz, CD3OD) δ 8.51-8.57 (m, 1H), 8.28 (s, 1H), 7.19 (s, 1H), 6.93-7.08 (m, 2H) 5.46 (s, 0.5 H), 5.33 (s, 0.5H), 5.02-5.10 (m, 1H), 4.16-4.52 (m, 4H), 4.03-4.13 (m, 1H), 3.83-3.99 (m, 3H), 3.62- 3.75 (m, 1H), 3.49-3.60 (m, 2H), 3.21 (s, 1 H), 2.31- 2.62 (m, 4H), 2.06-2.27 (m, 3H), 1.94-2.04 (m, 1H), 1.44-1.58 (m, 3H). |
| 552a | 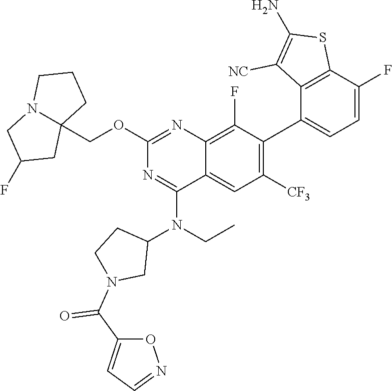 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2ccno2)C1] | |
| 553 | 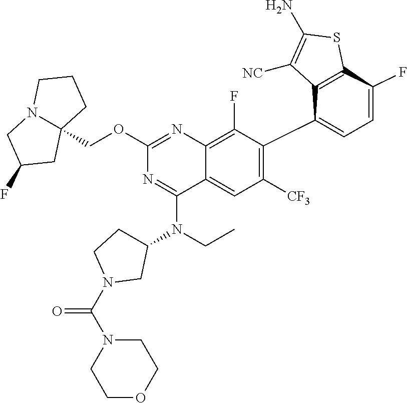 | LCMS m/z [M + 1]: 789.27 1H NMR (400 MHz, DMSO) δ 8.27 (s, 1H), 7.28- 7.20 (m, 1H), 7.08-7.00 (m, 1H), 5.35 (s, 0.5H), 5.26 (s, 0.5H), 5.05-4.97 (m, 1H), 4.38-4.21 (m, 2H), 3.96-3.92 (m, 3H), 3.78-3.53 (m, 7H), 3.48-3.35 (m, 3H), 3.29-3.14 (m, 4H), 3.09-2.99 (m, 1H), 2.45-2.12 (m, 5H), 2.06-1.87 (m, 3H), 1.51-1.39 (m, 3H). |
| 553a | 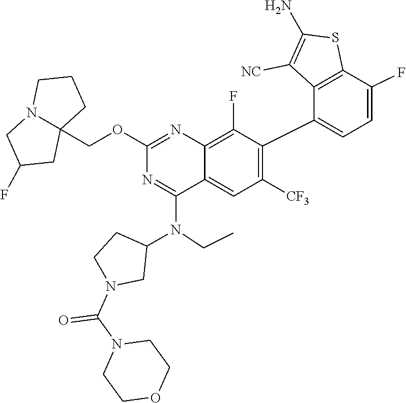 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2CCOCC2)C1] | |
| 554 | 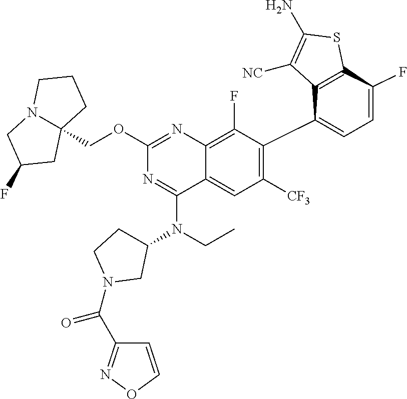 | LCMS m/z [M + 1]: 771.22 1HNMR (400 MHz, CD3OD) δ 8.82-8.80 (m, 1H), 8.46 (s, 1H), 8.29-8.28 (m, 1H), 7.23 (s, 1H) 7.04- 6.99 (m, 1H), 6.82 (s, 1H), 5.51 (s, 0.5H), 5.38 (s, 0.5H), 5.04-4.85 (m, 1H), 4.54-4.11 (m, 4H), 4.11- 3.88 (m, 4H), 3.87-3.63 (m, 4H), 2.45-2.26 (m, 4H), 2.24-2.17 (m, 3H), 2.01-1.93 (m, 1H), 1.49-1.28 (m, 3H). |
| 554a | 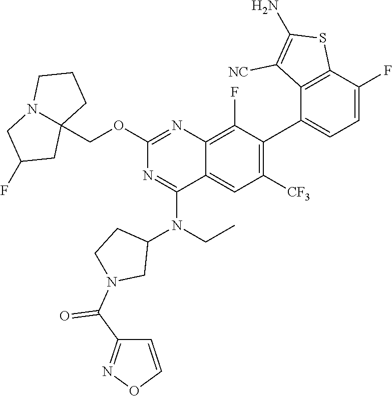 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2ccon2)C1] | |
| 555 | 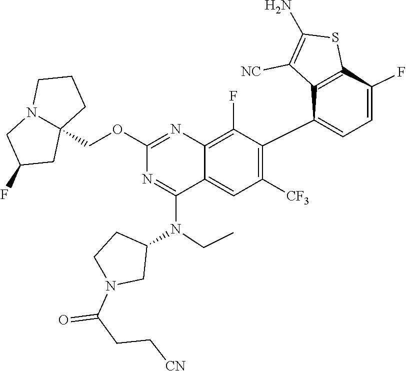 | LCMS m/z [M + 1]: 757.3 1H NMR (400 MHz, CD3OD) δ 8.51 (brs, 1H), 8.26 (s, 1H), 7.22 (t, J = 8.4 Hz, 1 H), 7.02 (t, J = 9.2 Hz, 1 H)), 5.21 (d, J = 90.8 Hz, 1 H), 5.08-5.01 (m, 1H), 4.46-4.33 (m, 2H), 4.08-4.01 (m, 1H), 3.94-3.85 (m, 3H), 3.75-3.60 (m, 2H), 3.53-3.44 (m, 3H), 3.17 (s, 1H), 2.82-2.73 (m, 4H), 2.59-1.97 (m, 8H), 1.50-1.45 (m, 3 H). |
| 555a | 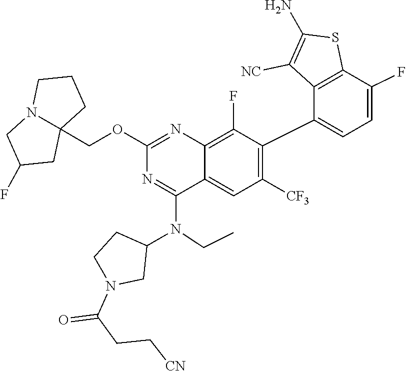 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)CCC#N)C1] | |
| 556 | 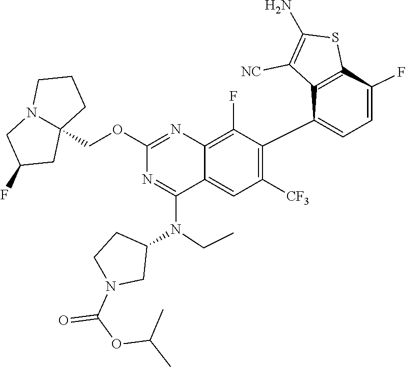 | LCMS m/z [M + 1]: 762.3 1H NMR (400 MHz, CD3OD) δ 8.24 (s, 1H), 7.22 (t, J = 7.6 Hz, 1 H), 7.01 (t, J = 8.4 Hz, 1 H)), 5.34 (d, J = 53.6 Hz, 1 H), 5.04-5.00 (m, 1H), 4.37-4.30 (m, 2H), 3.94-3.85 (m, 3H), 3.44-3.31 (m, 4H), 3.07 (s, 1H), 2.44-1.92 (m, 8H), 1.45 (t, J = 6.4 Hz, 3 H). |
| 556a | 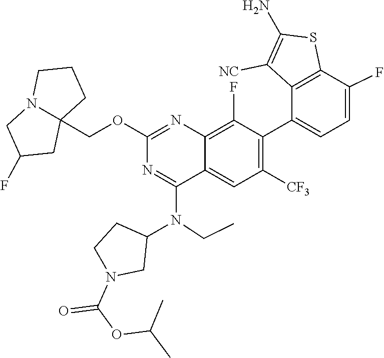 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC(C)C)C1] | |
| 557 | 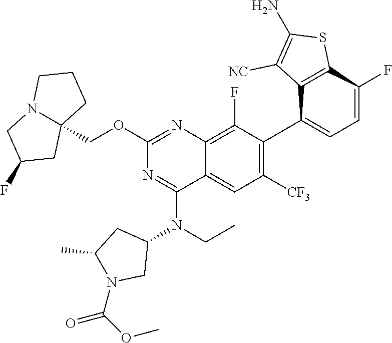 | LCMS m/z [M + 1]: 748.2 1H NMR (400 MHz, CD3OD) δ 8.23 (s, 1H), 7.23- 7.19 (m, 1H), 7.00 (t, 1H), 5.38 (s, 0.5H), 5.25 (s, 0.5H), 4.37-4.24 (m, 2H), 4.16-4.08 (m, 1H), 3.99- 3.82 (m, 3H), 3.70 (s, 3H), 3.51 (t, 1H), 3.41-3.32 (m, 1H), 3.29-3.22 (m, 2H), 3.08-3.00 (m, 1H), 2.62- 3.54 (m, 1H), 2.41-1.84 (m, 8H), 1.46-1.37 (m, 6H). |
| 558 | 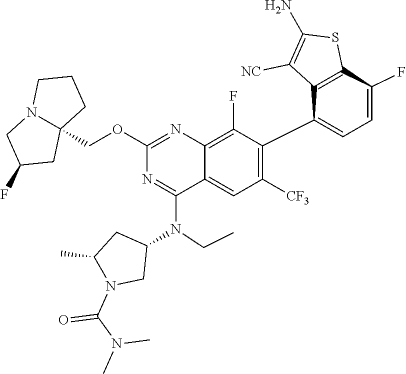 | LCMS m/z [M + 1]: 761.3 1H NMR (400 MHz, CD3OD) δ 8.23 (s, 1H), 7.23- 7.19 (m, 1H), 7.00 (t, 1H), 5.37 (s, 0.5H), 5.24 (s, 0.5H), 4.33 (d, 1H), 4.25-4.14 (m, 2H), 4.10-4.05 (m, 1H), 3.95-3.84 (m, 2H), 3.81-3.75 (m, 1H), 3.69-3.62 (m, 1H), 3.27-3.21 (m, 2H), 3.07-2.99 (m, 1H), 2.87 (s, 6H), 2.51-2.44 (m, 1H), 2.37-1.85 (m, 8H), 1.46 (t, 3H), 1.29 (d, 3H). |
| 559 | 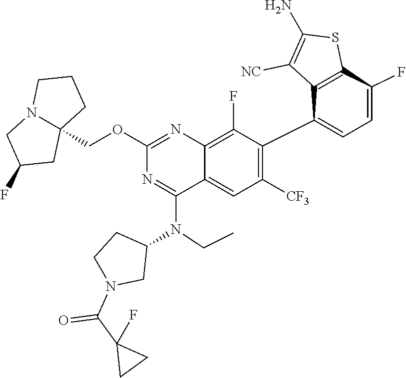 | LCMS m/z [M + 1]: 762.3 1H NMR (400 MHz, CD3OD) δ 8.25 (s, 1H), 7.20- 7.23 (m, 1H), 7.01 (t, 1H), 5.37 (s, 0.5H), 5.24 (s, 0.5H), 5.00-5.09 (m, 1H), 4.22-4.39 (m, 3H), 3.84- 4.09 (m, 4H), 3.47-3.71 (m, 1H), 3.23-3.25 (m, 3H), 3.00-3.06 (m, 1H), 2.22-2.55 (m, 4H), 2.11-2.18 (m, 1H), 1.87-2.02 (m, 3H), 1.45-1.48 (m, 3H), 1.32-1.39 (m, 2H), 1.21-1.26 (m, 2H). |
| 559a | 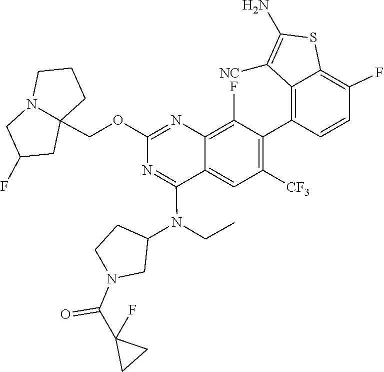 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2(F)CC2)C1] | |
| 560 | 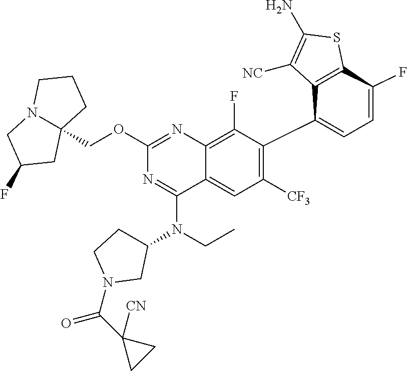 | LCMS m/z [M + 1]: 769.2 1H NMR (400 MHz, CD3OD) δ 8.25 (d, 1H), 7.20- 7.23 (m, 1H), 7.01 (t, 1H), 5.36 (s, 0.5H), 5.22 (s, 0.5H), 5.04-5.11 (m, 1H), 4.23-4.51 (m, 3H), 3.85- 4.03 (m, 4H), 3.47-3.69 (m, 1H), 3.16-3.25 (m, 3H), 2.98-3.05 (m, 1H), 2.21-2.59 (m, 4H), 2.12-2.17 (m, 1H), 1.87-2.01 (m, 3H), 1.67-1.71 (m, 1H), 1.45-1.56 (m, 6H). |
| 560a | 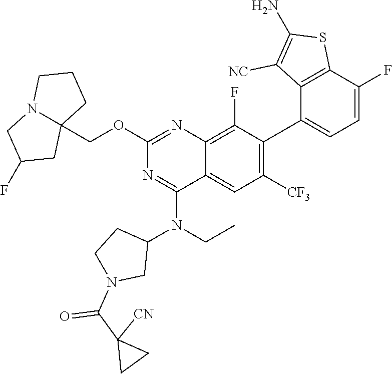 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2(C#N)CC2)C1] | |
| 561 | 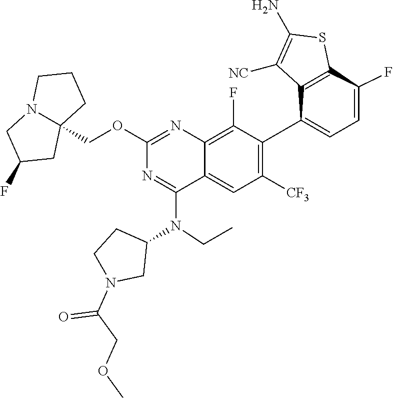 | LCMS m/z [M + 1]: 748.2 1H NMR (400 MHz, CD3OD) δ 8.23 (s, 1H), 7.19- 7.23 (m, 1H), 7.01 (t, 1H), 5.38 (s, 0.5H), 5.24 (s, 0.5H), 4.95-5.06 (m, 1H), 4.20-4.33 (m, 2H), 4.00- 4.16 (m, 3H), 3.84-3.92 (m, 3H), 3.54-3.70 (m, 2H), 3.41-3.48 (m, 3H), 3.34-3.36 (m, 1H), 3.20-3.24 (m, 2H), 3.01-3.07 (m, 1H), 2.34-2.54 (m, 2H), 2.13-2.28 (m, 3H), 1.86-2.02 (m, 3H), 1.43-1.48 (m, 3H). |
| 561a | 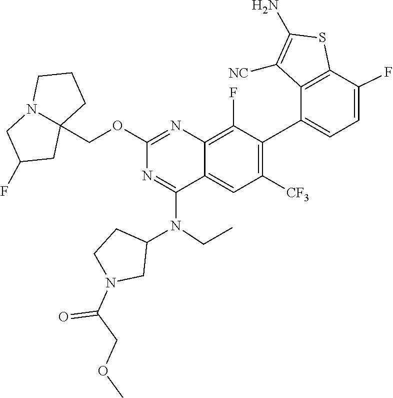 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)COC)C1] | |
| 562 | 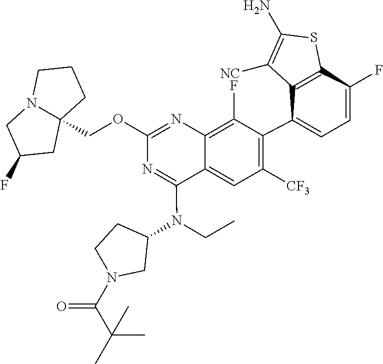 | LCMS m/z [M + 1]: 760.3 1H NMR (400 MHz, CD3CD) δ 8.25 (S, 1H), 7.23- 7.20 (m, 1H), 7.01 (t, 1H), 5.37 (s, 0.5H), 5.23 (s, 0.5H), 5.03-4.99 (m, 1H), 4.33 (d, 1H), 4.24 (d, 1H), 3.87-6.84 (m, 2H), 3.74-3.46 (m, 3H), 3.29-3.20 (m, 3H), 3.06-2.96 (m, 1H), 2.45-2.20 (m, 4H), 2.16-2.10 (m, 1H), 2.06-1.95 (m, 1H), 1.93-1.83 (m, 1H), 1.44 (t, 3H), 1.29 (s, 9H). |
| 562a | 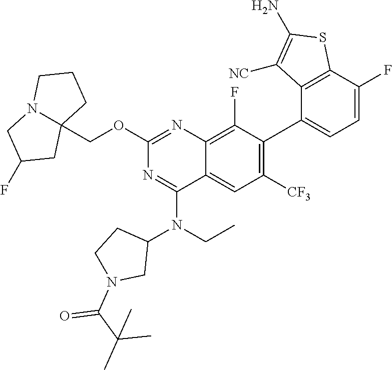 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C(C)(C)C)C1] | |
| 563 | 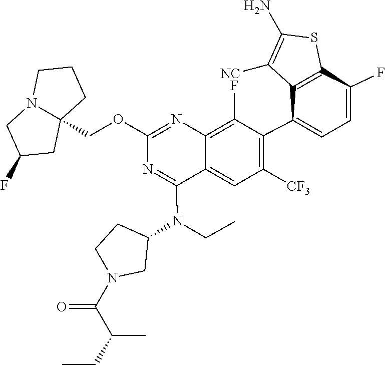 | LCMS m/z [M + 1]: 760.2 1H NMR (400 MHz, CD3OD) δ 8.25 (s, 1H), 7.25- 7.17 (m, 1H), 7.01 (t, 1H), 5.41 (s, 0.5H), 5.27 (s, 0.5H), 5.11-5.01 (m, 1H), 4.42 (m, 3H), 4.06-3.79 (m, 4H), 3.75-3.56 (m, 2H), 3.48-3.35 (m, 2H), 3.12- 3.03 (m, 1H), 2.71-2.58 (m, 1H), 2.58-2.14 (m, 6H), 2.10-1.98 (m, 2H), 1.96-1.87 (m, 1H), 1.73-1.64 (m, 1H), 1.50-1.40 (m, 3H), 1.19-1.03 (m, 3H), 1.00-0.86 (m, 3H). |
| 563a | 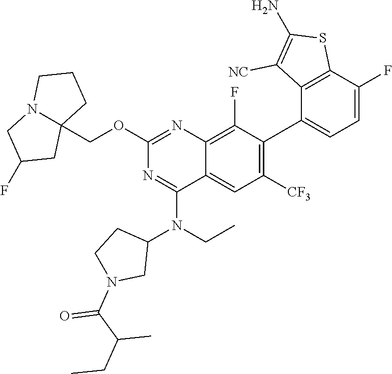 [SMILES: CCC(C)C(=O)N1CCC(N(CC)c2nc(OCC34CCCN3CC(F)C4)nc3c(F)c(-c4ccc(F)c5sc(N)c(C#N)c45)c(C(F)(F)F)cc23)C1] | |
| 564 | 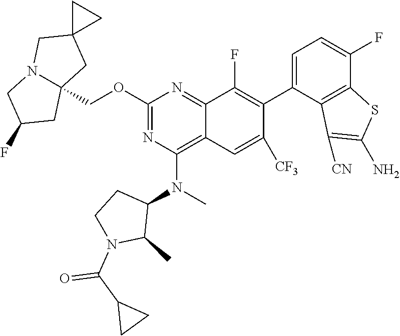 [SMILES: C[C@@H]1[C@H](N(C)c2nc(OC[C@]34C[C@@H](F)CN3CC3(CC3)C4)nc3c(F)c(-c4ccc(F)c5sc(N)c(C#N)c45)c(C(F)(F)F)cc23)CCN1C(=O)C1CC1] | LCMS m/z [M + 1]: 770.3 1HNMR (400 MHz, CD3OD) δ 8.36 (s,1H), 7.20- 7.23 (m, 1H), 7.01 (t, J = 8.64 Hz, 1H), 6.62-6.70 (m, 1H), 6.26-6.36 (m, 1H), 5.75-5.78 (m, 1H), 5.30-5.47 (m, 1H), 5.02-5.09 (m, 1H), 4.82-4.92 (m, 1H), 4.43- 4.59 (m, 2H). 3.75-3.98 (m, 1H), 3.53-3.63 (m, 4H). 3.32-3.38 (m, 3H), 2.31-2.92 (m, 6H). 1.71-1.83 (m, 1H), 1.10-1.17 (m, 3H). 0.57-0.61 (m, 4H). |
| 564a | 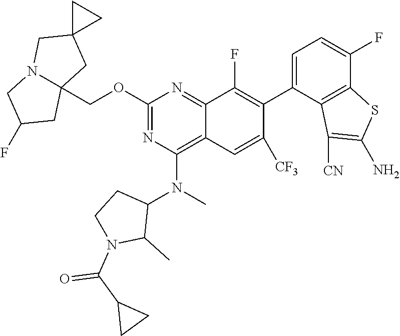 [SMILES: CC1C(N(C)c2nc(OCC34CC(F)CN3CC3(CC3)C4)nc3c(F)c(-c4ccc(F)c5sc(N)c(C#N)c45)c(C(F)(F)F)cc23)CCN1C(=O)C1CC1] | |
| 565 | 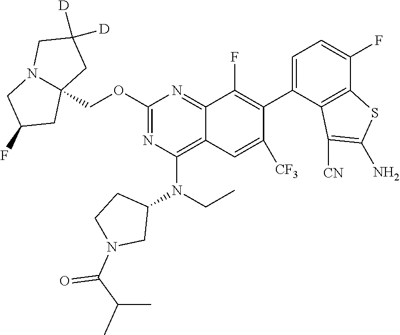 [SMILES: [2H]C1([2H])CN2C[C@H](F)C[C@]2(COc2nc(N(CC)[C@H]3CCN(C(=O)C(C)C)C3)c3cc(C(F)(F)F)c(-c4ccc(F)c5sc(N)c(C#N)c45)c(F)c3n2)C1] | LCMS m/z [M + 1]: 748.3 1HNMR (400 MHz, CD3OD) δ 8.27 (s, 1H), 7.21- 7.24 (m, 1H), 7.02 (t, J = 9.2 Hz, 1H), 5.44 (d, J = 52.8 Hz, 1H), 5.07-5.17 (m, 1H), 4.40-4.57 (m, 2H), 3.41-4.16 (m, 10H), 2.73-2.87 (m, 1H), 2.13-2.56 (m, 6H), 1.44-1.54 (m, 3H), 1.11-1.16 (m, 6H). |
| 565a | 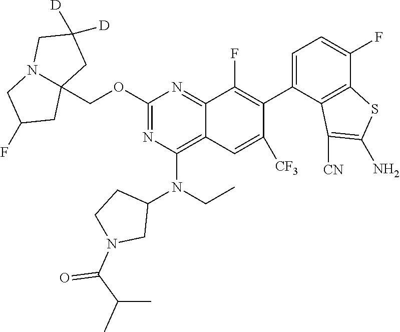 [SMILES: [2H]C1([2H])CN2CC(F)CC2(COc2nc(N(CC)C3CCN(C(=O)C(C)C)C3)c3cc(C(F)(F)F)c(-c4ccc(F)c5sc(N)c(C#N)c45)c(F)c3n2)C1] | |
| 566 | 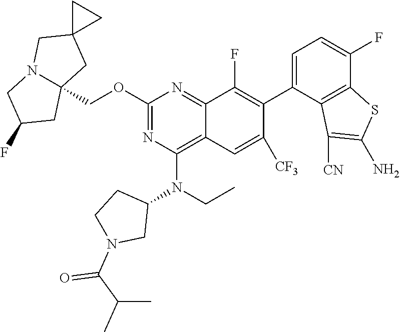 [SMILES: CCN(c1nc(OC[C@]23C[C@@H](F)CN2CC2(CC2)C3)nc2c(F)c(-c3ccc(F)c4sc(N)c(C#N)c34)c(C(F)(F)F)cc12)[C@H]1CCN(C(=O)C(C)C)C1] | LCMS m/z [M + 1]: 772.3 1HNMR (400 MHz, CD3OD) δ 8.32 (s, 1H), 7.22- 7.34 (m, 1H), 7.03 (t, J = 8.9 Hz, 1H), 5.61 (d, J = 51.6 Hz, 1H), 5.07-5.15 (m, 1H), 4.61-4.88 (m, 2H), 3.64-4.12 (m, 8H), 3.43-3.58 (m, 2H), 2.35-2.90 (m, 6H), 1.93-2.11 (m, 1H), 1.47-1.53 (m, 3H), 1.11-1.16 (m, 6H), 0.57-0.85 (m, 4H). |
| 566a | 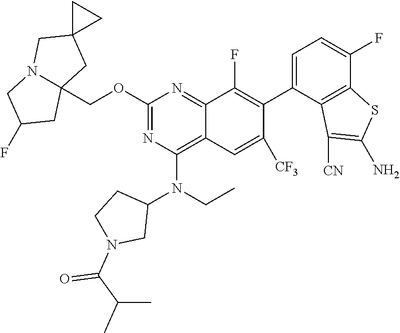 [SMILES: CCN(c1nc(OCC23CC(F)CN2CC2(CC2)C3)nc2c(F)c(-c3ccc(F)c4sc(N)c(C#N)c34)c(C(F)(F)F)cc12)C1CCN(C(=O)C(C)C)C1] | |
| 567 | 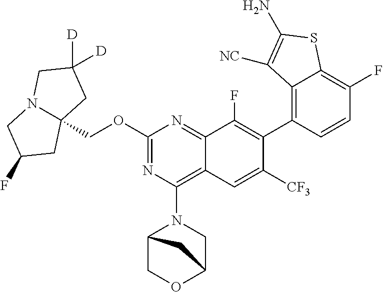 [SMILES: [2H]C1([2H])CN2C[C@H](F)C[C@]2(COc2nc(N3C[C@H]4C[C@@H]3CO4)c3cc(C(F)(F)F)c(-c4ccc(F)c5sc(N)c(C#N)c45)c(F)c3n2)C1] | LCMS m/z [M + 1]: 663.2 1H NMR (400 MHz, CD3OD-d4): δ 8.32 (s, 1H), 7.19-7.23 (m, 1H), 7.02 (t, J = 8.62 Hz, 1H), 5.42-5.54 (m, 2H), 4.84 (s, 1H), 4.48-4.62 (m, 2H), 4.31 (d, J = 9.52 Hz, 1H), 3.62-4.08 (m, 6H), 3.33 (s, 1H), 2.03- 2.65 (m, 6H). |
| 567a | 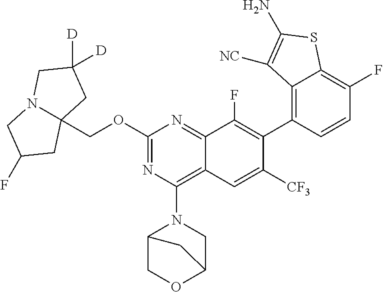 [SMILES: [2H]C1([2H])CN2CC(F)CC2(COc2nc(N3CC4CC3CO4)c3cc(C(F)(F)F)c(-c4ccc(F)c5sc(N)c(C#N)c45)c(F)c3n2)C1] | |
| 568 | 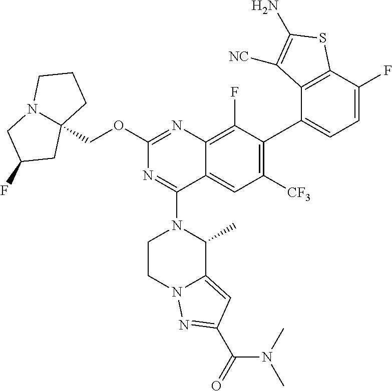 | LCMS m/z [M + 1]: 770.2 1H NMR (400 MHz, CD3OD) δ 8.27 (d, J = 5 Hz, 1H), 7.23-7.26 (m, 1H), 7.04 (t, J = 8.9 Hz, 1H), 6.53- 6.54 (m, 1H), 5.92-5.99 (m, 1H), 5.56 (d, J = 52 Hz, 1H), 4.42-4.91 (m, 5H), 3.85-4.19 (m, 4H), 3.43-3.51 (m, 1H), 3.38 (s, 3H), 3.09 (s, 3H), 2.16-2.76 (m, 6H), 1.93 (d, J = 6.72 Hz, 3H). |
| 568a | 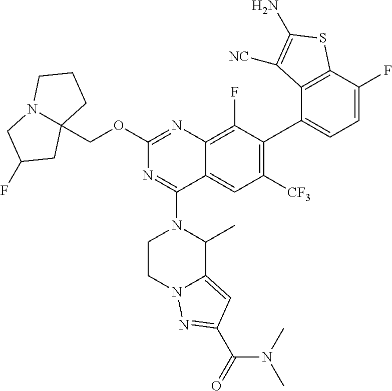 | |
| 570 | 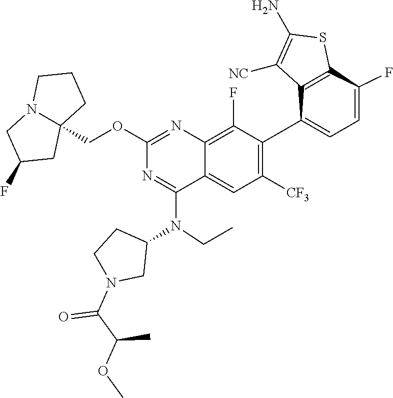 | LCMS m/z [M + 1]: 762.2 1H NMR (400 MHz, CD3OD) δ 8.25 (s, 1H), 7.20- 7.24 (m, 1H), 6.99-7.04 (m, 1H), 5.38 (s, 0.5H), 5.25 (s, 0.5H), 5.00-5.08 (m, 1H), 4.08-4.36 (m, 4H), 3.78-3.93 (m, 4H), 3.34-3.70 (m, 4H), 3.23 (s, 3H), 3.05 (s, 1H), 2.12-2.52 (m, 5H), 1.87-2.02 (m, 3H), 1.42-1.45 (m, 3H), 1.28-1.35 (m, 3H). |
| 571 | 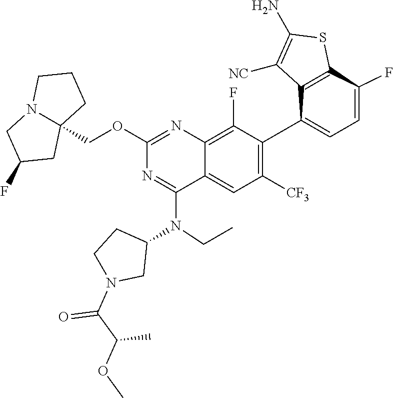 | LCMS m/z [M + 1]: 762.2 1H NMR (400 MHz, CD3OD) δ 8.24 (s, 1H), 7.20- 7.23 (m, 1H), 7.01 (t, 1H), 5.37 (s, 0.5H), 5.24 (s, 0.5H), 5.00-5.08 (m, 1H), 3.81-4.37 (m, 7H), 3.48- 3.69 (m, 2H), 3.32-3.34 (m, 3H), 3.21-3.24 (m, 3H), 3.00-3.05 (m, 1H), 2.11-2.53 (m, 5H), 1.86-2.00 (m, 3H), 1.42-1.47 (m, 3H), 1.34 (t, 3H). |
| 571a | 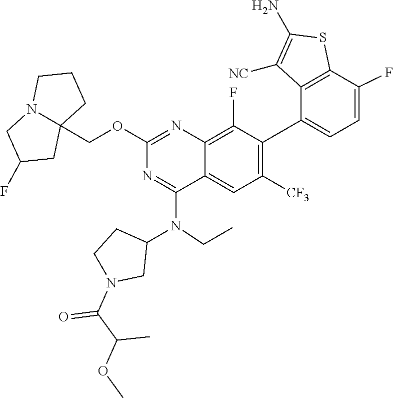 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C(C)OC)C1] | |
| 572 | 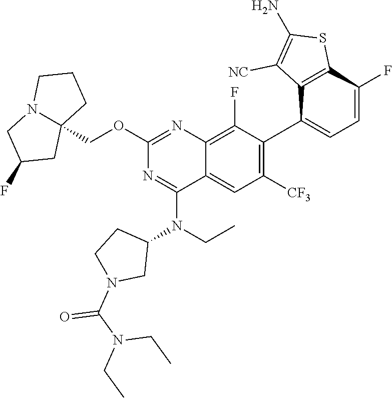 | LCMS m/z [M + 1]: 775.29 1H NMR (400 MHz, DMSO) δ 8.28 (s, 1H), 7.29- 7.23 (m, 1H), 7.05 (t, J = 9.2 Hz, 1H), 5.39 (s, 0.5H), 5.26 (s, 0.5H), 5.08-4.99 (m, 1H), 4.38-4.24 (m, 2H), 3.94-3.83 (m, 3H), 3.69-3.55 (m, 3H), 3.33-3.16 (m, 7H), 3.08-2.99 (m,1H), 2.44-2.22 (m, 4H), 2.20-2.12 (m, 1H), 2.05-1.88 (m, 3H), 1.48 (t, J = 6.8 Hz, 3H), 1.20 (t, J = 6.8 Hz, 3H). |
| 572a | 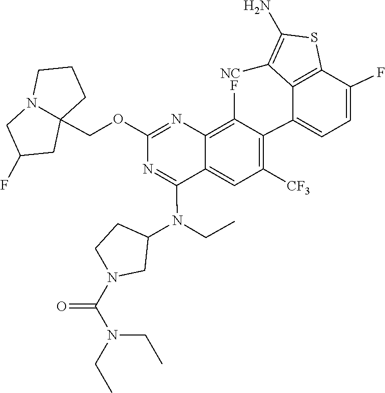 [SMILES: CCN(CC)C(=O)N1CCC(N(CC)c2nc(OCC34CCCN3CC(F)C4)nc3c(F)c(-c4ccc(F)c5sc(N)c(C#N)c45)c(C(F)(F)F)cc23)C1] | |
| 573 | 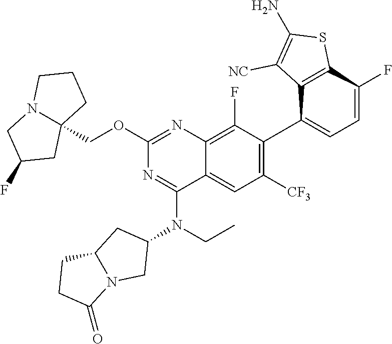 | LCMS m/z [M + 1]: 730.1 1H NMR (400 MHz, CD3OD) δ 8.19 (s, 1H), 7.20- 7.22 (m, 1H), 6.98-7.03 (m, 1H), 5.36 (s, 0.5H), 5.23 (s, 0.5H), 5.13 (m, 1H), 4.11-4.26 (m, 4H), 3.79-3.87 (m, 3H), 3.53-3.56 (m, 1H), 3.19-3.23 (m, 2H), 3.01 (s, 1H), 2.66-2.75 (m, 3H), 2.12-2.41 (m, 5H), 1.88- 1.99 (m, 4H), 1.46-1.49 (m, 3H). |
| 574 | 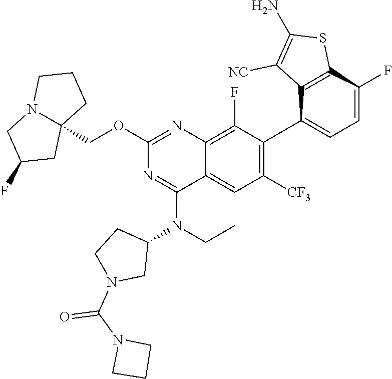 | LCMS m/z [M + 1]: 759.1 1H NMR (400 MHz, CD3OD) δ 8.17 (s, 1H), 7.23- 7.21 (m, 1H), 7.04-7.00 (m, 1H), 5.60 (s, 0.5H), 5.47 (s, 0.5H), 4.69-4.59 (m, 2H), 4.28-4.24 (m, 1H), 4.00-3.86 (m, 6H), 3.41 (s, 1H), 3.13 (s, 1H), 2.59- 2.16 (m, 8H), 1.81-1.68 (m, 6H). |
| 574a | 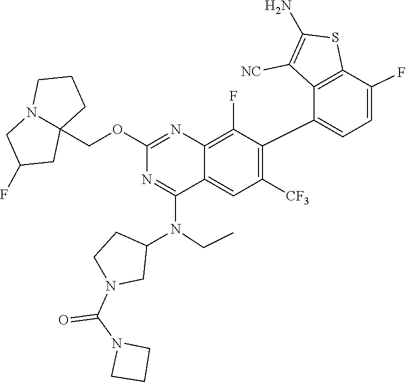 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2CCC2)C1] | |
| 575 | 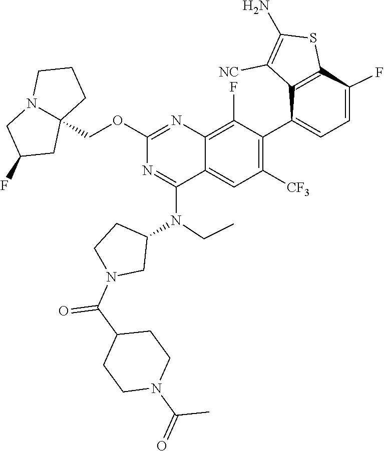 | LCMS m/z [M + 1]: 829.3 1HNMR (400 MHz, CD3OD) δ 8.25 (d, 1H), 7.23- 7.20 (m, 1H), 7.01 (t, 1H), 5.39 (s, 0.5H), 5.26 (s, 0.5H), 5.13-4.98 (m, 1H), 4.59-4.48 (m, 3H), 4.02- 3.58 (m, 6H), 3.51-3.31 (m, 2H), 3.27-3.12 (m, 2H), 3.10-3.02 (m, 1H), 2.90-2.66 (m, 2H), 2.60-2.14 (m, 5H), 2.10 (d, 3H), 2.07-1.98 (m, 2H), 1.95-1.76 (m, 3H), 1.74-1.55 (m, 2H), 1.51-1.39 (m, 3H). |
| 575a | 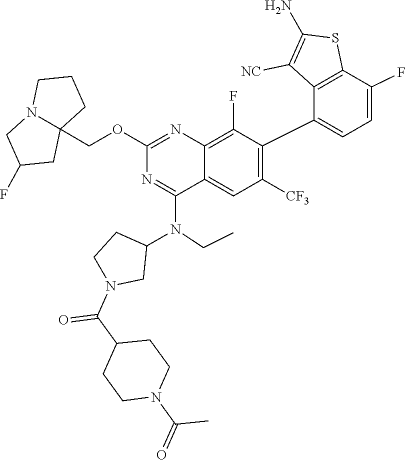 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CCN(C(C)=O)CC2)C1] | |
| 576 | 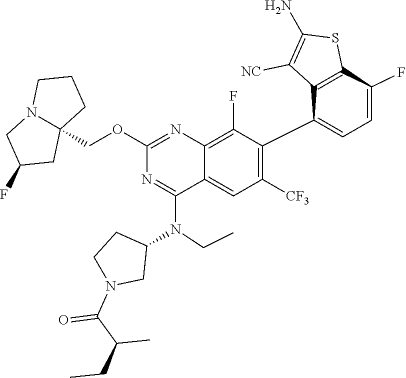 | LCMS m/z [M + 1]: 760.20 1H NMR (400 MHz, CD3OD) δ 8.24 (s, 1H), 7.21 (t, 1H), 7.01 (t, 1H), 5.39 (s, 0.5H), 5.26 (s, 0.5H), 5.08- 5.03 (m, 1H), 4.34-4.16 (m, 3H), 4.02 (m, 1H), 3.91- 3.83 (m, 3H), 3.70-3.59 (m, 2H), 3.47-3.34 (m, 2H), 3.07 (s, 1H), 2.66-2.15 (m, 7H), 2.03-1.92 (m, 3H), 1.70-1.65 (m, 1H), 1.47-1.42 (m, 2H), 1.12 (t, 3H), 0.93-0.83 (m, 3H). |
| 577 | 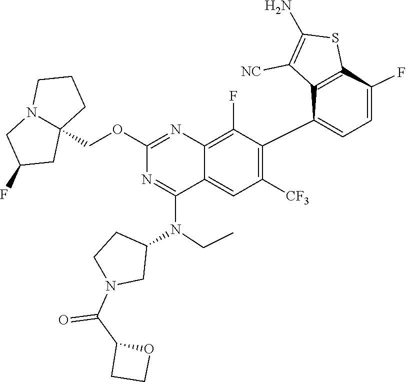 | LCMS m/z [M + 1]: 760.20 1H NMR (400 MHz, CD3OD) δ 8.23 (d, 1H), 7.21 (m, 1H), 7.01 (t, 1H), 5.44-5.39 (m, 1.5H), 5.27 (s, 0.5H), 4.98 (m, 1H), 4.72-4.70 (m, 1H), 4.59-4.58 (m, 1H), 4.36-4.25 (m, 2H), 3.99 (m, 1H), 3.86-3.82 (m, 2H), 3.74-3.55 (m, 2H), 3.47 (m, 1H), 3.35 (s, 3H), 3.09 (m, 1H), 2.98-2.96 (m, 1H), 2.84 (m, 1H), 2.52-2.28 (m, 4H).2.16 (m, 1H), 2.04 (s, 2H), 1.93 (m, 2H), 1.48-1.43 (m, 3H). |
| 577a | 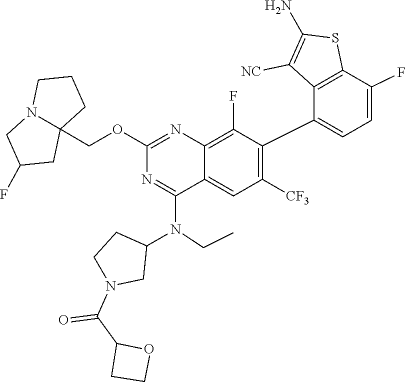 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CCO2)C1] | |
| 578 | 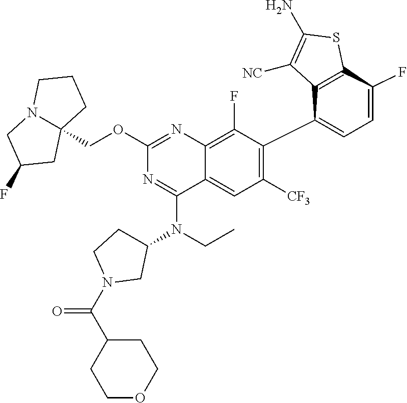 | LCMS m/z [M + 1]: 788.20 1H NMR (400 MHz, CD3OD) δ 8.24 (s, 1H), 7.21 (s, 1H), 7.01 (t, 1H), 5.38 (s, 0.5H), 5.24 (s, 0.5H), 5.04 (s, 1H), 4.39-4.10 (m, 3H), 4.02-3.78(m, 5H),3.63 (m, 2H), 3.50-3.38 (m, 3H), 3.25 (m, 2H), 3.05 (s, 1H), 2.82 (s, 1H), 2.44-2.14 (m, 5H), 2.01-1.68 (m, 7H), 1.43 (m, 3H). |
| 578a | 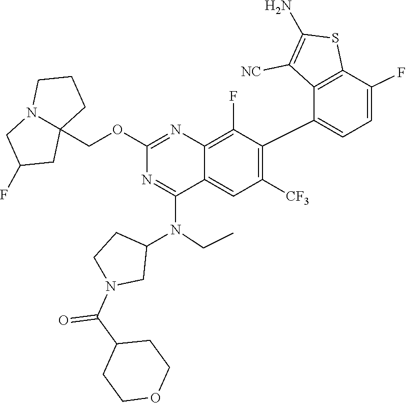 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CCOCC2)C1] | |
| 579 | 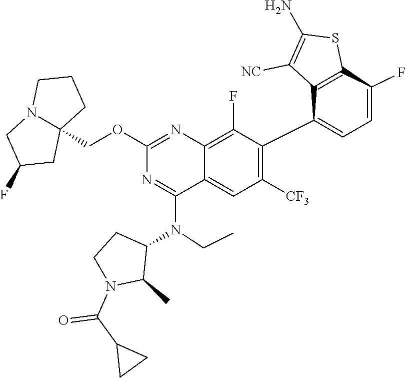 | LCMS m/z [M + 1]: 758.3 1H NMR (400 MHz, CD3OD) δ 8.24 (s, 1H), 7.26- 7.18 (m, 1H), 7.01 (t, 1H), 5.45 (s, 0.5H), 5.32 (s, 0.5H), 4.72-4.67 (m, 1H), 4.57-4.29 (m, 3H), 4.17- 4.04 (m, 1H), 3.90-3.78 (m, 2H), 3.59-3.41 (m, 3H), 3.26-3.09 (m, 2H), 2.60-2.29 (m, 4H), 2.23-1.85 (m, 5H), 1.51-1.40 (m, 3H), 1.37-1.27 (m, 3H), 0.92-0.78 (m, 4H). |
| 580 | 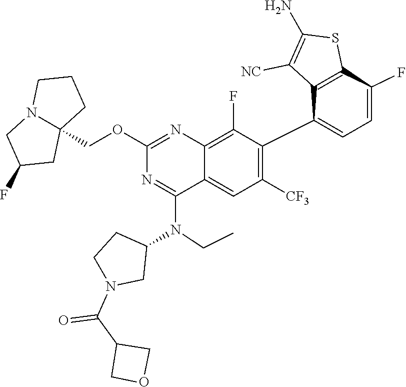 | LCMS m/z [M + 1]: 760.2 1H NMR (400 MHz, CD3OD) δ 8.23 (s, 1H), 7.19- 7.22 (m, 1H), 7.01 (t, 1H), 5.37 (s, 0.5H), 5.24 (s, 0.5H), 4.96-5.04 (m, 1H), 4.85-4.88 (m, 4H), 4.00- 4.32 (m, 3H), 3.80-3.91 (m, 3H), 3.42-3.67 (m, 3H), 3.21-3.27 (m, 3H), 2.99-3.06 (m, 1H), 2.11-2.52 (m, 5H), 1.87-2.02 (m, 3H), 1.42-1.46 (m, 3H). |
| 580a | 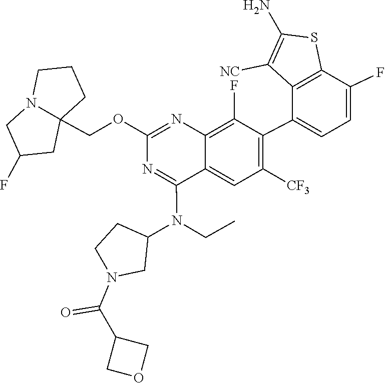 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2COC2)C1] | |
| 581 | 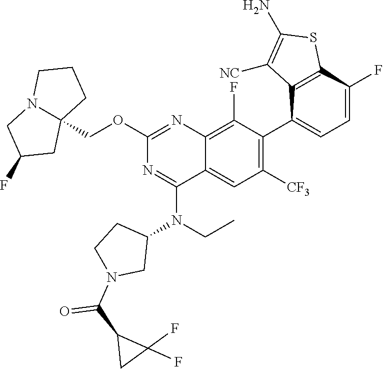 | LCMS m/z [M + 1]: 780.2 1H NMR (400 MHz, CD3OD) δ 8.29 (d, 1H), 7.22 (s, 1H), 7.02 (t, 1H), 5.54 (s, 0.5H), 5.41 (s, 0.5H), 5.03-5.14 (m, 1H), 4.59-4.62 (m, 1H), 4.44-4.51 (m, 1H), 4.16-4.21 (t, 0.5H), 3.76-4.06 (m, 5H), 3.64- 3.69 (m, 3H), 3.45-3.52 (m, 0.5H), 2.62-2.90 (m, 1H), 2.57-2.64 (m, 1H), 2.42-2.47 (m, 3H), 2.30-2.34 (m, 1H), 2.22 (s, 2H), 2.08 (s, 2H), 1.93 (s, 1H), 1.80 (s, 1H), 1.45-1.53 (m, 3H). |
| 582 | 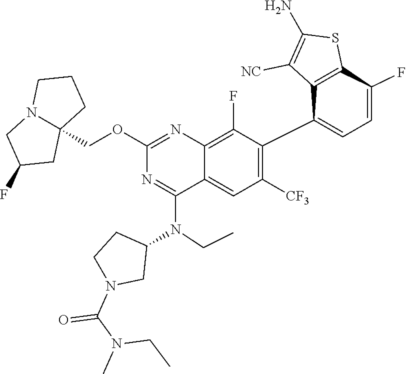 | LCMS m/z [M + 1]: 761.3 1H NMR (400 MHz, CD3OD) δ 8.27 (s, 1H), 7.26- 7.23 (m, 1H), 7.06-7.02 (m, 1H), 5.39 (s, 0.50H), 5.26 (s, 0.50H), 5.04-4.99 (m, 1H), 4.36-4.25 (m, 2H), 3.91-3.83 (m, 3H), 3.69-3.53 (m, 3H), 3.32-3.23 (m, 5H), 3.04-3.01 (m, 1H), 2.90 (s, 3H), 2.44-2.13 (m, 5H), 2.03-1.89 (m, 3H), 1.49-1.46 (m, 3H), 1.22- 1.18 (m, 3H). |
| 582a | 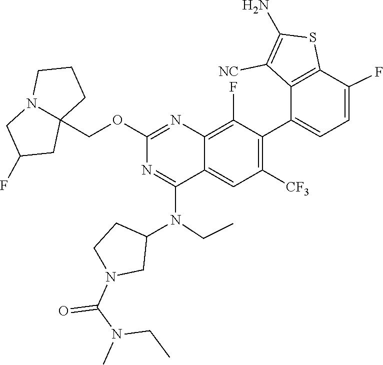 [SMILES: CCN(C)C(=O)N1CCC(N(CC)c2nc(OCC34CCCN3CC(F)C4)nc3c(F)c(-c4ccc(F)c5sc(N)c(C#N)c45)c(C(F)(F)F)cc23)C1] | |
| 583 | 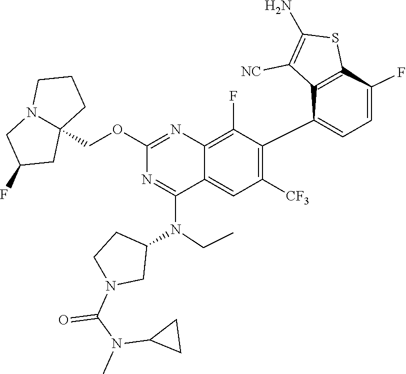 | LCMS m/z [M + 1]: 773.3 1H NMR (400 MHz, CD3OD) δ 8.24 (s, 1H), 7.23- 7.19 (m, 1H), 7.03-6.98 (m, 1H), 5.36 (s, 0.54H), 5.22 (s, 0.51H), 5.03-4.99 (m, 1H), 4.32-4.22 (m, 2H), 3.92-3.83 (m, 3H), 3.70-3.66 (m, 2H), 3.59-3.57 (m, 1H), 3.23-3.18 (m, 3H), 3.03-2.97 (m, 1H), 2.87 (s, 3H), 2.71 (s, 1H), 2.41-2.10 (m, 5H), 2.00-1.86 (m, 3H), 1.45-1.42 (m, 3H), 0.82-0.66 (m, 4H). |
| 583a | 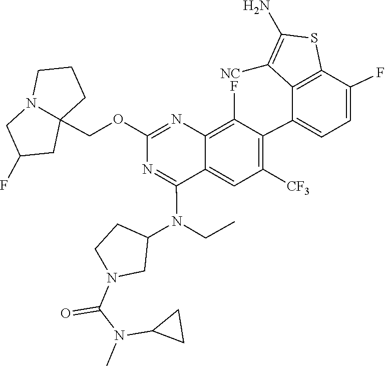 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N(C)C2CC2)C1] | |
| 584 | 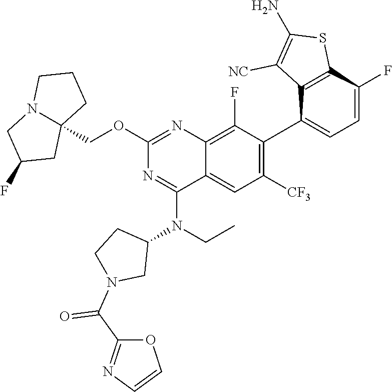 | LCMS m/z [M + 1]: 771.1 1HNMR (400 MHz, CD3OD) δ 8.52 (s, 1H), 8.27 (s, 1H), 8.05-8.14 (m, 1H), 7.35-7.46 (m, 1H), 7.22 (s, 1H), 7.69-7.07 (m, 1 H), 5.42 (s, 0.5 H), 5.29 (s, 0.5H), 4.99-5.11 (m, 1H), 4.55-4.72 (m, 1H), 4.28- 4.59 (m, 2H), 3.80-4.27 (m, 5H), 3.61-3.76 (m, 1H), 3.39-3.50 (m, 2H), 3.13 (s, 1 H), 2.37-2.72 (m, 3H), 1.98-2.37 (m, 5H), 1.51 (s, 3H). |
| 584a | 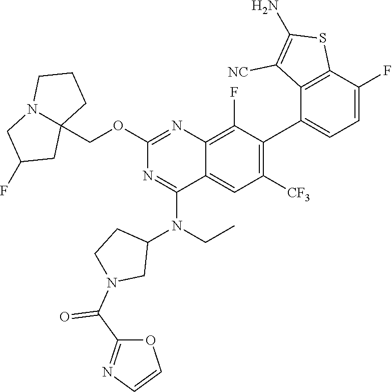 | |
| 585 | 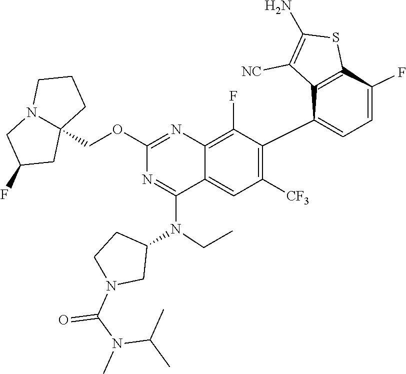 | LCMS m/z [M + 1]: 775.3 1H NMR (400 MHz, CD3OD) δ = 8.26 (s, 1H), 7.25- 7.19 (m, 1H), 7.01 (t, J = 8.9, 1H), 5.44 (s, 0.5H), 5.31 (s, 0.5H), 5.06-4.98 (m, 1H), 4.39 (dd, J = 39.6, 11.2, 2H), 4.16-4.08 (m, 1H), 3.95-3.77 (m, 3H), 3.67-3.37 (m, 6H), 3.13 (m, 1H), 2.75 (s, 3H), 2.44-1.92 (m, 8H), 1.46 (t, J = 6.8, 3H), 1.17 (t, J = 6.4, 6H). |
| 585a | 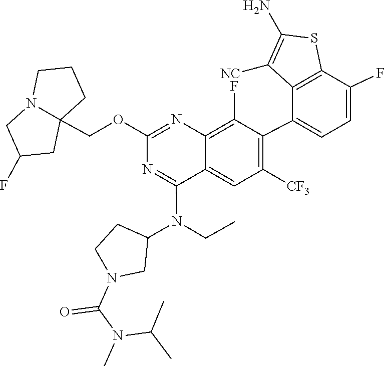 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N(C)C(C)C)C1] | |
| 586 | 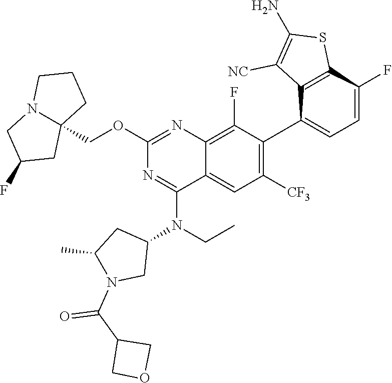 | LCMS m/z [M + 1]: 774.2 1H NMR (400 MHz, CD3OD) δ 8.23 (s, 1H), 7.22- 7.19 (m, 1H), 7.01 (t, 1H), 5.39 (s, 0.5H), 5.25 (s, 0.5H), 4.89-4.84 (m, 2H), 4.81-4.72 (m, 2H), 4.36 (t, 1H), 4.24 (d, 1H), 4.19-4.06 (m, 2H), 3.90-3.75 (m, 3H), 3.56 (t, 1H), 3.27 (s, 1H), 3.10-3.02 (m, 1H), 2.66-2.55 (m, 1H), 2.32-1.87 (m, 8H), 1.49-1.38 (m, 6H). |
| 587 | 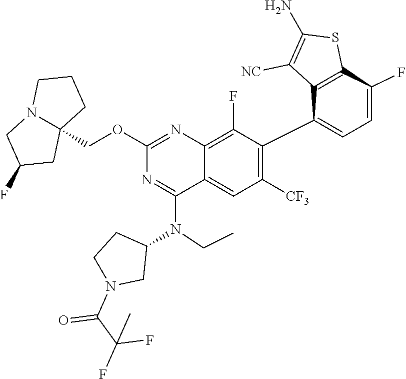 | LCMS m/z [M + 1]: 768.2 1H NMR (400 MHz, CD3OD) δ 8.25 (s, 1H), 7.25- 7.18 (m, 1H), 7.01 (t, 1H), 5.39 (s, 0.5H), 5.25 (s, 0.5H), 5.07-4.96 (m, 1H), 4.43-4.02 (m, 4H), 3.95- 3.69 (m, 4H), 3.64-3.36 (m, 2H), 3.27-3.25 (m, 1H), 3.10-3.01 (m, 1H), 2.61-1.98 (m, 8H), 1.89-1.76 (m, 3H), 1.54-1.40 (m, 3H). |
| 587a | 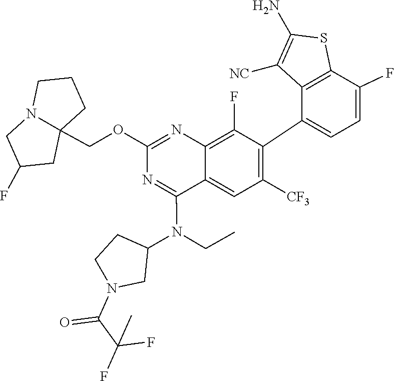 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C(C)(F)F)C1] | |
| 588 | 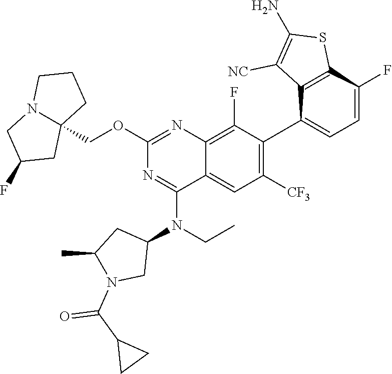 | LCMS m/z [M + 1]: 758.3 1H NMR (400 MHz, CD3OD) δ 8.25 (s, 1H), 7.21 (s, 1H), 7.03-6.99 (m, 1H), 5.36 (s, 0.57H), 5.23 (s, 0.58H), 4.97-4.67 (m, 1H), 4.31-4.25 (m, 3H), 4.10 (s, 1H), 3.90-3.85 (m, 3H), 3.48-3.43 (m, 1H), 3.23- 3.18 (m, 2H), 3.00 (s, 1H), 2.61 (s, 1H), 2.37-2.12 (m, 4H), 1.99-1.81 (m, 4H), 1.56 (s, 1H), 1.43 (s, 5H), 0.99 (s, 1H), 0.83-0.79 (m, 3H). |
| 589 | 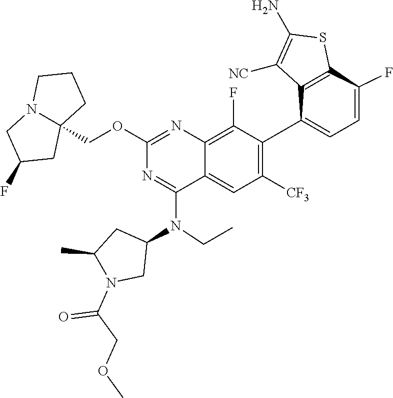 | LCMS m/z [M + 1]: 762.4 1H NMR (400 MHz, CD3OD) δ 8.23 (s, 1H), 7.23- 7.20 (m, 1H), 7.03-6.99 (m, 1H), 5.38 (s, 0.60H), 5.25 (s, 0.62H), 4.56-3.83 (m, 9H), 3.66 (s, 1H), 3.40 (s, 3H), 3.24 (s, 2H), 3.04 (s, 1H), 2.64-2.61 (m, 1H), 2.38-2.02 (m, 8H), 1.46-1.35 (m, 6H). |
| 590 | 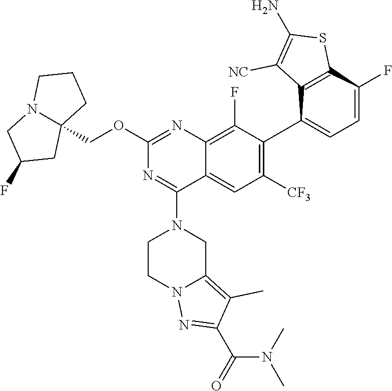 | LCMS m/z [M + 1]: 770.24 1H NMR (400 MHz, CD3OD) δ 8.23 (s, 1H), 7.23 (m, 1H), 7.06-6.95 (m, 1H), 5.36 (s, 0.5H), 5.23 (s, 0.5H), 5.10 (m, 2H), 4.56-4.22 (m, 6H), 3.29-2.92 (m, 10H), 2.40-1.82 (m, 9H). |
| 591 | 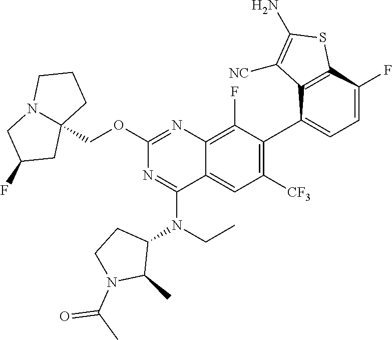 | LCMS m/z [M + 1]: 732.3 1H NMR (400 MHz, CD3OD) δ 8.24 (s, 1H), 7.26- 7.19 (m, 1H), 7.02 (t, 1H), 5.51 (s, 0.5H), 5.37 (s, 0.5H), 4.65-4.58 (m, 1H), 4.55-4.38 (m, 3H), 3.97- 3.54 (m, 7H), 3.48-3.36 (m, 1H), 2.59-2.38 (m, 4H), 2.33-2.16 (m, 3H), 2.14-1.96 (m, 4H), 1.52-1.27 (m, 6H) |
| 592 | 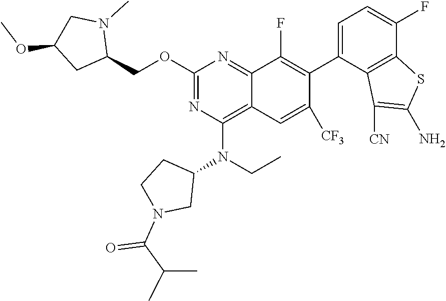 [SMILES: CCN(c1nc(OC[C@H]2C[C@@H](OC)CN2C)nc2c(F)c(-c3ccc(F)c4sc(N)c(C#N)c34)c(C(F)(F)F)cc12)[C@H]1CCN(C(=O)C(C)C)C1] | LCMS m/z [M + 1]: 732.3 1HNMR (400 MHz, CD3OD) δ 8.29 (s, 1H), 7.18- 7.28 (m, 1H), 7.03 (t, J = 8.9 Hz, 1H), 5.00-5.14 (m, 1H), 4.88-4.92 (m, 1H), 4.56-4.68 (m, 1H), 3.74-4.18 (m, 8H), 3.37-3.70 (m, 2H), 3.20-3.29 (m, 2H), 3.06- 3.17 (m, 4H), 2.73-2.90 (m, 1H), 2.31-2.66 (m, 3H), 2.08-2.18 (m, 1H), 1.42-1.57 (m, 3H), 1.07-1.19 (m, 6H). |
| 592a | 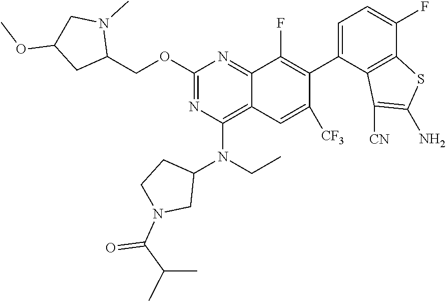 [SMILES: CCN(c1nc(OCC2CC(OC)CN2C)nc2c(F)c(-c3ccc(F)c4sc(N)c(C#N)c34)c(C(F)(F)F)cc12)C1CCN(C(=O)C(C)C)C1] | |
| 593 | 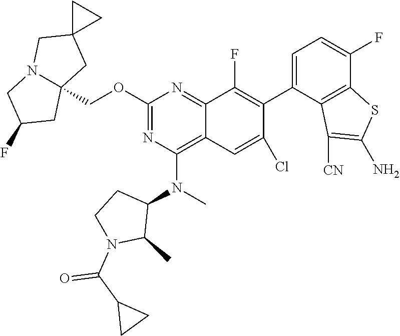 [SMILES: C[C@@H]1[C@H](N(C)c2nc(OC[C@]34C[C@@H](F)CN3CC3(CC3)C4)nc3c(F)c(-c4ccc(F)c5sc(N)c(C#N)c45)c(Cl)cc23)CCN1C(=O)C1CC1] | LCMS m/z [M + 1]: 736.3 1HNMR (400 MHz, CD3OD) δ 8.18-8.24 (m, 1H), 7.21-7.25 (m, 1H), 7.05 (t, J = 8.88 Hz, 1H), 5.60 (d, J = 51.54 Hz, 1H), 4.97-5.06 (m, 1H), 4.64-4.73 (m, 3H), 3.62-4.04 (m, 8H), 3.45-3.48 (m, 1H), 2.36-2.77 (m, 5H), 2.00-2.07 (m, 1H). 1.80-1.84 (m, 1H), 1.03- 1.24 (m, 3H), 0.65-0.69 (m, 8H). |
| 593a | 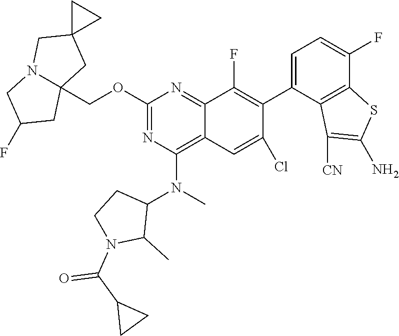 [SMILES: CC1C(N(C)c2nc(OCC34CC(F)CN3CC3(CC3)C4)nc3c(F)c(-c4ccc(F)c5sc(N)c(C#N)c45)c(Cl)cc23)CCN1C(=O)C1CC1] | |
| 594 | 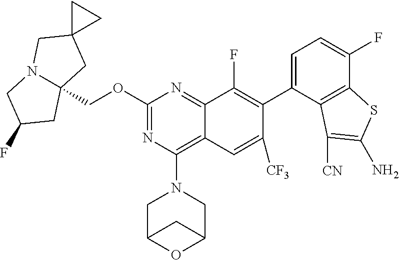 [SMILES: N#Cc1c(N)sc2c(F)ccc(-c3c(C(F)(F)F)cc4c(N5CC6CC(C5)O6)nc(OC[C@]56C[C@@H](F)CN5CC5(CC5)C6)nc4c3F)c12] | LCMS m/z [M + 1]: 687.3 1HNMR (400 MHz, CD3OD) δ 8.66-8.68 (m, 1H), 7.21-7.24 (m, 1H), 7.03 (t, J = 9.44 Hz, 1H), 5.60 (d, J = 50.94 Hz, 1H), 4.88-4.89 (m, 1H), 4.68-4.83 (m, 3H), 4.31-4.64 (m, 4H), 3.87-4.06 (m, 2H), 3.67-3.70 (m, 1H), 3.42-3.47 (m, 1H), 1.95-3.11 (m, 6H), 0.58- 0.85 (m, 4H). |
| 594a | 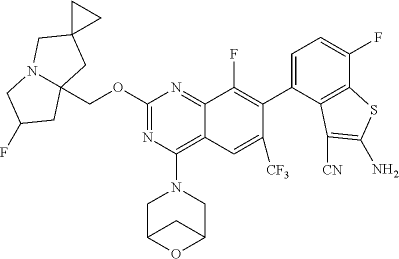 [SMILES: N#Cc1c(N)sc2c(F)ccc(-c3c(C(F)(F)F)cc4c(N5CC6CC(C5)O6)nc(OCC56CC(F)CN5CC5(CC5)C6)nc4c3F)c12] | |
| 595 | 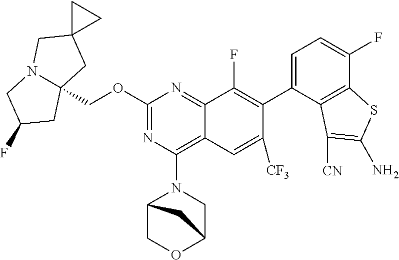 [SMILES: N#Cc1c(N)sc2c(F)ccc(-c3c(C(F)(F)F)cc4c(N5C[C@H]6C[C@@H]5CO6)nc(OC[C@]56C[C@@H](F)CN5CC5(CC5)C6)nc4c3F)c12] | LCMS m/z [M + 1]: 687.3 1HNMR (400 MHz, CD3OD) δ 8.35-8.36 (m, 1H), 7.20-7.23 (m, 1H), 7.01 (t, J = 8.28 Hz, 1H), 5.52- 5.66 (m, 2H), 4.87-4.89 (m, 1H), 4.61-4.76 (m, 2H), 4.32-4.35 (m, 1H), 3.86-4.07 (m, 5H), 3.66-3.69 (m, 1H), 3.41-3.47 (m, 1H). 2.54-2.66 (m, 3H), 2.03-2.17 (m, 3H). 0.53-0.84 (m, 4H). |
| 595a | 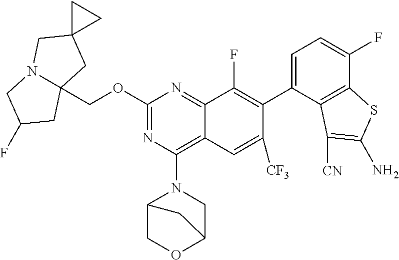 [SMILES: N#Cc1c(N)sc2c(F)ccc(-c3c(C(F)(F)F)cc4c(N5CC6CC5CO6)nc(OCC56CC(F)CN5CC5(CC5)C6)nc4c3F)c12] | |
| 596 | 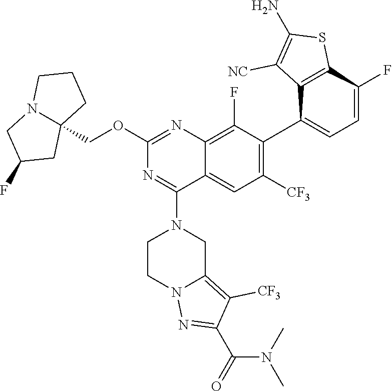 | LCMS m/z [M + 1]: 824.21 1H NMR (400 MHz, CD3OD) δ 8.29 (s, 1H), 7.03- 7.25 (m, 1H), 7.06 (t, J = 8.9Hz, 1H), 5.41 (s, 0.5H), 5.32 (s, 2H), 5.27 (s, 0.5H), 4.57-4.43 (m, 4H), 4.41- 4.29 (m, 2H), 3.30-3.00 (m, 10H), 2.42-2.23 (m, 2H), 2.21-2.13 (m, 1H), 2.08-1.90 (m, 3H). |
| 596a | 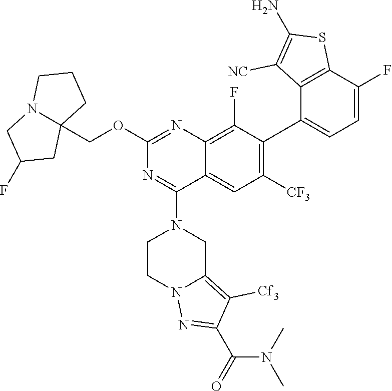 | |
| 597 | 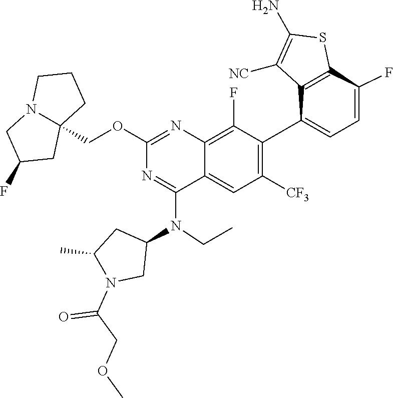 | LCMS m/z [M + 1]: 762.26 1H NMR (400 MHz, DMSO) δ 8.26 (s, 1H), 7.28- 7.23 (m, 1H), 7.05 (t, J = 8.8 Hz, 1H), 5.44 (s, 0.5H), 5.30 (s, 0.5H), 5.27-5.17 (m, 1H), 4.57-4.74 (m, 1H), 4.45-4.24 (m, 3H), 4.20-4.05 (m, 2H), 4.02-3.90 (m, 2H), 3.87-3.72 (m,2H), 3.50-3.41 (m, 3H), 3.03-3.26 (m, 1H), 3.13-3.03 (m, 1H), 2.75-2.63 (m, 1H), 2.45- 2.01 (m, 7H), 1.99-1.88 (m, 1H), 1.51-1.43 (m, 3H), 1.38-1.31 (m, 3H). |
| 598 | 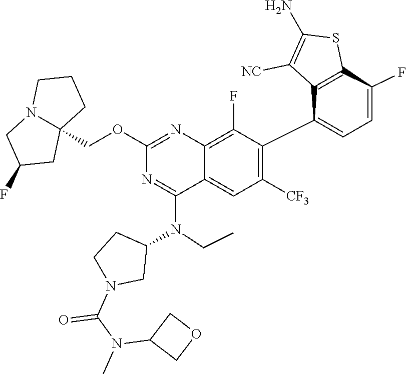 | LCMS m/z [M + 1]: 789.3 1H NMR (400 MHz, CD3OD) δ 8.29 (s, 1H), 7.34- 7.19 (m, 1H), 7.10-6.97 (m, 1H), 5.41 (s, 0.54H), 5.28 (s, 0.55H), 5.10-5.01 (m, 1H), 4.85-4.64 (m, 5H), 4.45-4.21 (m, 2H), 4.00-3.84 (m, 3H), 3.72-3.54 (m, 3H), 3.33-3.23 (m, 3H), 3.13-3.02 (m, 1H), 2.95 (s, 3H), 2.48-2.14 (m, 5H), 2.08-1.89 (m, 3H), 1.53- 1.45 (m, 3H). |
| 598a | 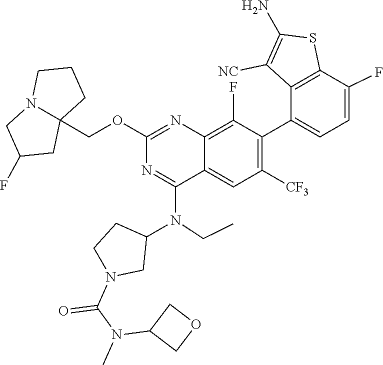 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N(C)C2COC2)C1] | |
| 599 | 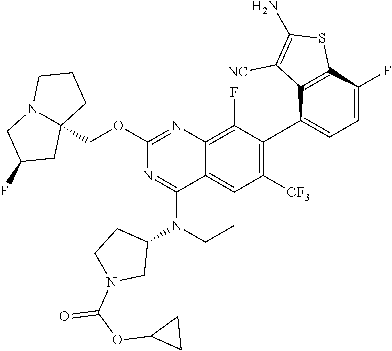 | LCMS m/z [M + 1]: 760.2 1H NMR (400 MHz, CD3OD) δ 8.26 (s, 1H), 7.35- 7.18 (m, 1H), 7.11-6.99 (m, 1H), 5.42 (s, 0.55H), 5.29 (s, 0.52H), 5.08-4.97 (m, 1H), 4.39-4.24 (m, 2H), 4.16-4.05 (m, 1H), 4.00-3.84 (m, 3H), 3.82-3.43 (m, 3H), 3.32-3.20 (m, 3H), 3.12-3.00 (m, 1H), 2.52- 2.15 (m, 5H), 2.08-1.89 (m, 3H), 1.52-1.44 (m, 3H), 0.73 (s, 4H). |
| 599a | 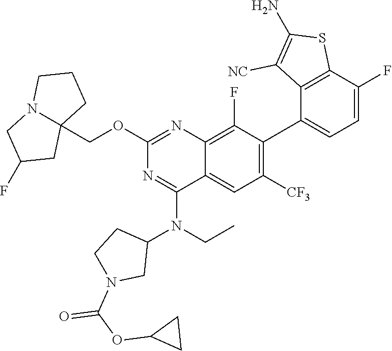 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2CC2)C1] | |
| 600 | 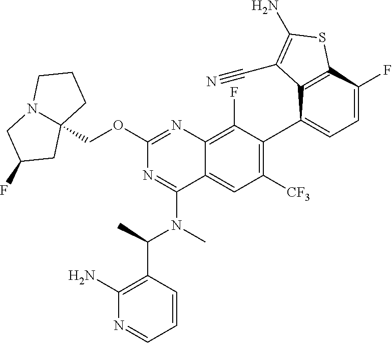 | LCMS m/z [M + 1]: 713.2 1H NMR (400 MHz, CD3OD) δ 8.39 (s, 1H), 7.96 (d, 1H), 7.77 (d, 1H), 7.20 (t, 1H), 7.01 (t, 1H), 6.79 (t, 1H), 6.20-6.18 (m,1H), 5.56 (s, 0.5H), 5.43 (s, 0.5H), 4.83 (s, 1H), 4.65-4.54 (m, 2H), 3.93-3.73 (m, 3H), 3.47-3.32 (m, 4H), 2.65-2.26 (m, 5H), 2.08 (m, 1H), 1.76 (d, 3H). |
| 601 | 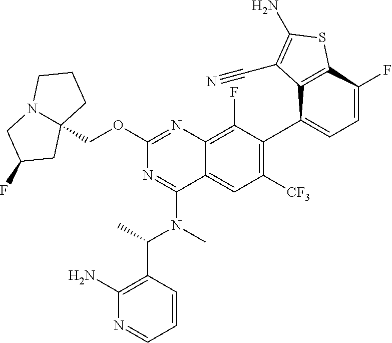 | LCMS m/z [M + 1]: 713.1 1H NMR (400 MHz, CD3OD) δ 8.40 (s, 1H), 7.96 (d, 1H), 7.75 (d, 1H), 7.23-7.19 (m, 1H), 7.01 (t, 1H), 6.78 (t, 1H), 6.19-6.18 (m, 1H), 5.57 (s, 0.5H), 5.44 (s, 0.5H), 4.83 (s, 1H), 4.66 (d, 1H), 4.48 (d, 1H), 3.90- 3.79 (m, 1H), 3.77-3.73 (m, 2 H), 3.37 (s, 4H), 2.60- 2.55 (m, 1H), 2.49 (m, 1H), 2.35-2.33 (m, 1H), 2.26- 2.24 (m, 2H), 2.05 (s,1H), 1.74 (d, 3H). |
| 601a | 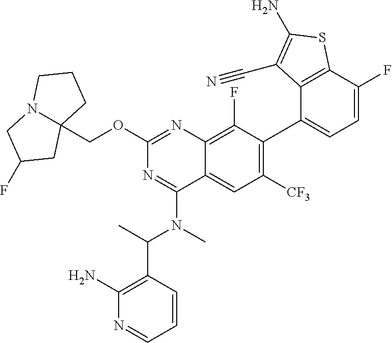 [SMILES: CC(c1cccnc1N)N(C)c1nc(OCC23CCCN2CC(F)C3)nc2c(F)c(-c3ccc(F)c4sc(N)c(C#N)c34)c(C(F)(F)F)cc12] | |
| 602 | 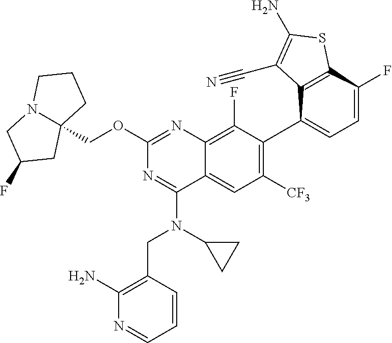 | LCMS m/z [M + 1]: 725.1 1H NMR (400 MHz, CD3OD) δ 9.02 (s, 1H), 8.21 (s, 1H), 7.91 (d, 1H), 7.47 (d, 1H), 7.25-7.22 (m, 1H), 7.02 (t, 1H), 6.68 (t, 1H), 5.55 (s, 0.5H), 5.42 (s, 0.5H), 5.06-4.93 (m, 2H), 4.48 (d, 1H), 4.34 (d, 1H), 3.94-3.74 (m, 3H), 3.65-3.57 (m, 1H), 3.42-3.34 (m, 1H), 2.55-2.42 (m, 2H), 2.31-2.15 (m, 3H), 2.06-1.97 (m, 1H), 1.18-1.07 (m, 2H), 1.00-0.88 (m, 2H). |
| 602a | 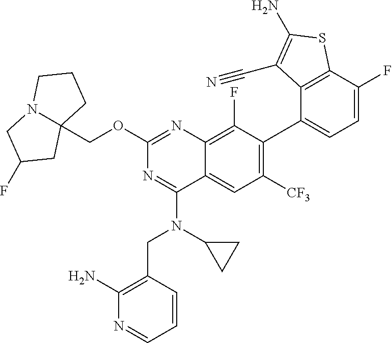 [SMILES: N#Cc1c(N)sc2c(F)ccc(-c3c(C(F)(F)F)cc4c(N(Cc5cccnc5N)C5CC5)nc(OCC56CCCN5CC(F)C6)nc4c3F)c12] | |
| 603 | 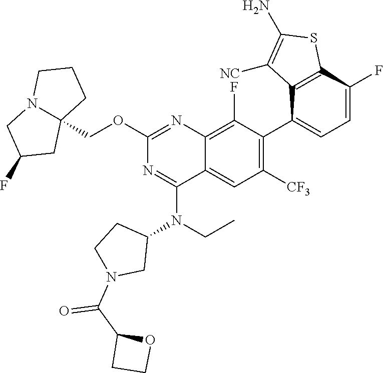 | LCMS m/z [M + 1]: 760.2 1H NMR (400 MHz, CD3OD) δ 8.23 (s, 1H), 7.19- 7.23 (m, 1H), 7.00 (t, 1H), 5.47 (t, 0.5H), 5.36-5.39 (m, 1H), 5.24 (s, 0.5H), 4.93-5.05 (m, 1H), 4.67-4.76 (m, 1H), 4.56-4.63 (m, 1H), 4.22-4.23 (m, 2H), 3.98- 4.11 (m, 1H), 3.63-3.92 (m, 4H), 3.33-3.53 (m, 2H), 3.24-3.26 (m, 2H), 2.78-3.06 (m, 3H), 2.33-2.54 (m, 2H), 2.08-2.28 (m, 3H), 1.88-2.02 (m, 3H), 1.45 (t, 3H). |
| 604 | 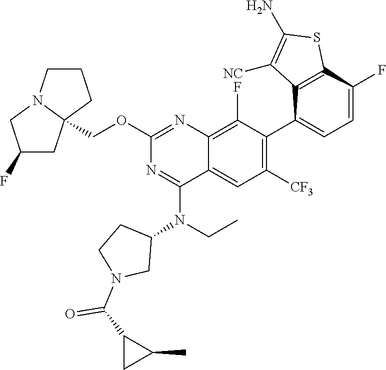 | LCMS m/z [M + 1]: 758.2 1H NMR (400 MHz, CD3OD) δ 8.2 (d, 1H), 7.20- 7.23 (m, 1H), 7.00 (t, 1H), 5.37 (t, 0.5H), 5.24 (s, 0.5H), 5.00-5.11 (m, 1H), 4.24-4.32 (m, 2H), 3.84- 4.07 (m, 4H), 3.36-3.77 (m, 3H), 3.18-3.26 (m, 3H), 2.99-3.05 (m, 1H), 2.10-2.60 (m, 5H), 1.88-2.00 (m, 3H), 1.55-1.57 (m, 1H), 1.43-1.50 (m, 3H), 1.11-1.16 (m, 3H), 1.05-1.08 (m, 1H), 0.66-0.72 (m, 1H). |
| 604a | 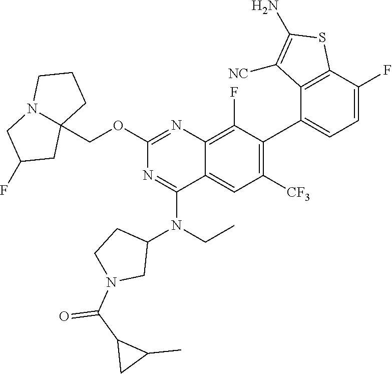 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CC2C)C1] | |
| 605 | 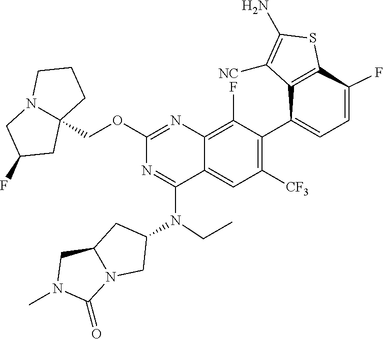 | LCMS m/z [M + 1]: 745.24 1H NMR (400 MHz, CD3OD) δ 8.16 (s, 1H), 7.29- 7.14 (m, 1H), 7.08-6.91 (m, 1H), 5.37 (s, 0.5H), 5.23 (s, 0.5H), 4.72-4.59 (m, 1H), 4.52-4.41 (m, 1H), 4.37-4.21 (m, 2H), 4.16-4.06 (m, 1H), 3.95-3.72 (m, 2H), 3.67-3.58 (m, 1H), 3.39-3.31 (m, 2H), 3.29-3.11 (m, 3H), 3.08-2.95 (m, 1H), 2.90-2.70 (m, 3H), 2.51- 1.82 (m, 8H), 1.52-1.38 (m, 3H). |
| 606 | 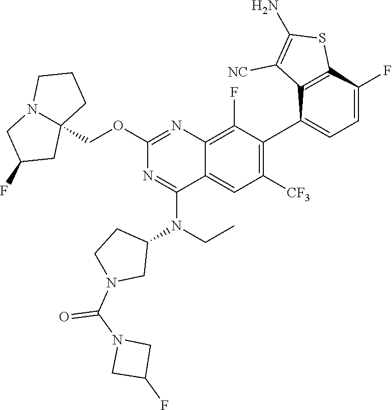 | LCMS m/z [M + 1]: 777.3 1H NMR (400 MHz, CD3OD) δ 8.28 (d, 1H), 7.23 (m, 1H), 7.03 (m, 1H), 6.81-6.88 (m, 0.5H), 6.60- 6.68 (m, 0.5H), 6.33 (d, 1H), 5.98-6.06 (m, 1H), 5.50-5.83 (m, 1H), 5.40 (s, 0.5H), 5.27 (s, 0.5H), 4.50-4.60 (m, 1H), 3.84-3.92 (m, 1H), 3.67-3.78 (m, 1H), 3.59-3.67 (m, 1H), 3.40 (d, 3H), 3.10-3.29 (m, 3H), 2.52-2.68 (m, 2H), 2.10-2.42 (m, 4H), 1.91-2.08 (m, 3H). |
| 606a | 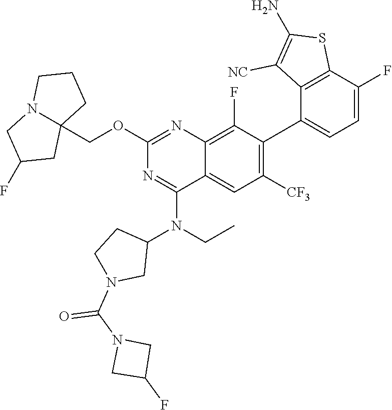 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2CC(F)C2)C1] | |
| 607 | 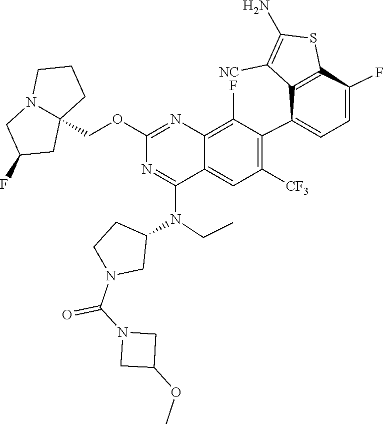 | LCMS m/z [M + 1]: 789.3 1H NMR (400 MHz, CD3OD) δ 8.28 (d, 1H), 7.23 (m, 1H), 7.03 (m, 1H), 6.81-6.88 (m, 0.5H), 6.60- 6.68 (m, 0.5H), 6.33 (d, 1H), 5.98-6.06 (m, 1H), 5.50-5.83 (m, 1H), 5.40 (s, 0.5H), 5.27 (s, 0.5H), 4.50-4.60 (m, 1H), 3.84-3.92 (m, 1H), 3.67-3.78 (m, 1H), 3.59-3.67 (m, 1H), 3.40 (d, 3H), 3.10-3.29 (m, 3H), 2.52-2.68 (m, 2H), 2.10-2.42 (m, 4H), 1.91-2.08 (m, 3H). |
| 607a | 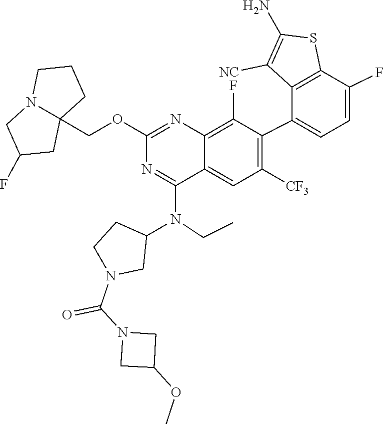 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2CC(OC)C2)C1] | |
| 608 | 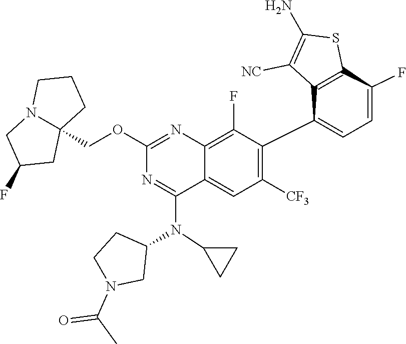 | LCMS m/z [M + 1]: 730.3 1H NMR (400 MHz, CD3OD) δ 8.70 (s, 1H), 7.22 (t, J = 7.6 Hz, 1 H), 7.01 (t, J = 9.2 Hz, 1 H)), 5.38 (d, J = 53.2 Hz, 1 H), 4.93-4.89 (m, 1H), 4.55-4.16 (m, 3H), 2.74-2.19 (m, 7H), 3.13 (s, 1H), 2.74-2.57 (m, 2H), 2.42-2.19 (m, 3H), 2.09-1.95 (m, 6H), 1.05 (s, 2 H), 0.72 (s, 2 H). |
| 608a | 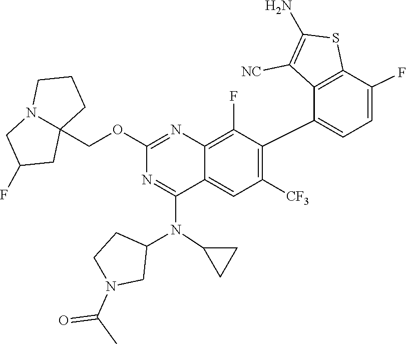 [SMILES: CC(=O)N1CCC(N(c2nc(OCC34CCCN3CC(F)C4)nc3c(F)c(-c4ccc(F)c5sc(N)c(C#N)c45)c(C(F)(F)F)cc23)C2CC2)C1] | |
| 609 | 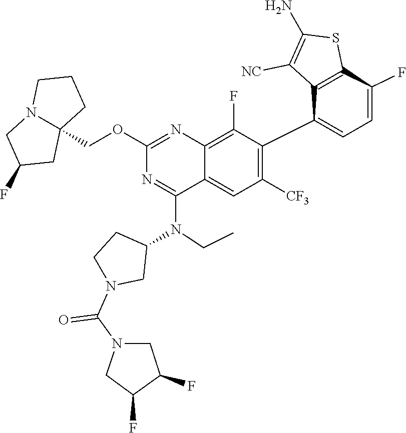 | LCMS m/z [M + 1]: 809.3 1H NMR (400 MHz, CD3OD) δ 8.28 (d, 1H), 7.23 (m, 1H), 7.03 (m, 1H), 6.81-6.88 (m, 0.5H), 6.60- 6.68 (m, 0.5H), 6.33 (d, 1H), 5.98-6.06 (m, 1H), 5.50-5.83 (m, 1H), 5.40 (s, 0.5H), 5.27 (s, 0.5H), 4.50-4.60 (m, 1H), 3.84-3.92 (m, 1H), 3.67-3.78 (m, 1H), 3.59-3.67 (m, 1H), 3.40 (d, 3H), 3.10-3.29 (m, 3H), 2.52-2.68 (m, 2H), 2.10-2.42 (m, 4H), 1.91-2.08 (m, 3H). |
| 609a | 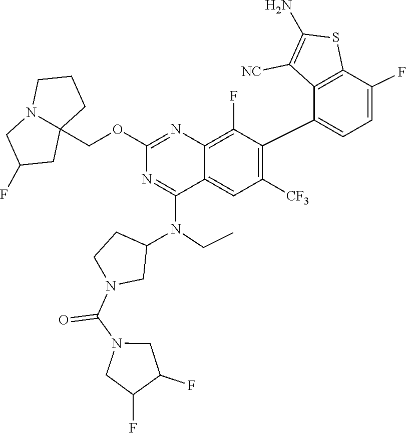 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2CC(F)C(F)C2)C1] | |
| 610 | 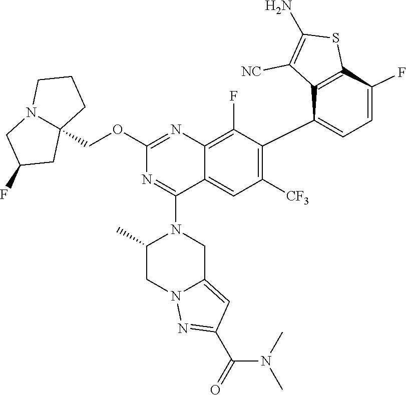 | LCMS m/z [M + 1]: 770.3 1H NMR (400 MHz, CD3OD) δ 8.45 (s, 1H), 8.25 (s, 1H), 7.26-7.20 (m, 1H), 7.02 (t, 1H), 6.56 (s, 1H), 5.52-5.40 (m, 2H), 5.39-5.35 (m, 1H), 5.24-5.18 (m, 1H), 4.66-4.54 (m, 2H), 4.51-4.45 (m, 1H), 4.36-4.30 (m, 1H), 3.75-3.65 (m, 1H), 3.63-3.46 (m, 3H), 3.29- 3.23 (m, 3H), 3.15-3.05 (m, 3H), 2.59-2.48 (m, 1H), 2.45-2.39 (m, 1H), 2.35-2.28 (m, 1H), 2.23-2.13 (m, 2H), 2.07-1.99 (m, 1H), 1.55-1.42 (m, 3H). |
| 611 | 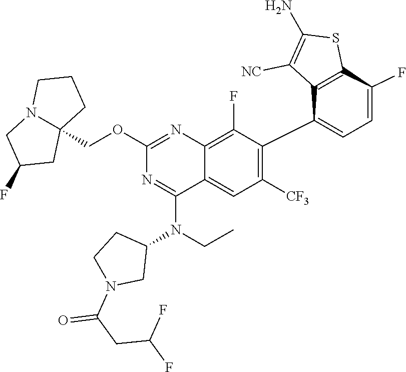 | LCMS m/z [M + 1]: 768.2 1H NMR (400 MHz, CD3OD) δ 8.26 (s, 1H), 7.27- 7.17 (m, 1H), 7.02 (m, 1H), 6.26 (m, 1H), 5.45 (s, 0.5H), 5.32 (s, 0.5H), 5.08-4.95 (m, 1H), 4.39 (m, 2H), 4.15-3.99 (m, 1H), 3.89 (m, 3H), 3.58 (m, 5H), 3.21-2.98 (m, 3H), 2.60-2.30 (m, 4H), 2.22 (s, 1H), 2.11 (s, 2H), 1.97 (s, 1H), 1.47 (m, 3H). |
| 611a | 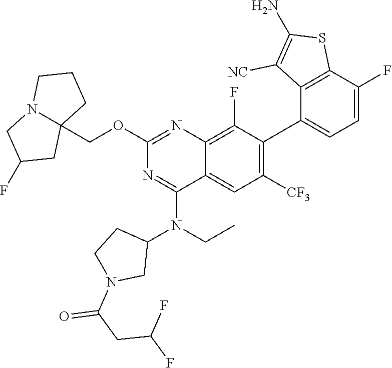 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)CC(F)F)C1] | |
| 612 | 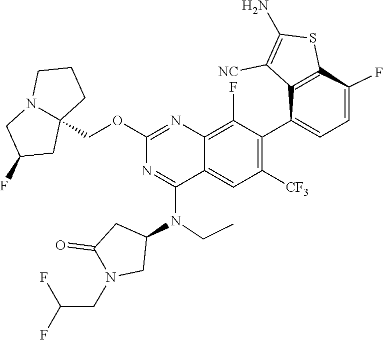 | LCMS m/z [M + 1]: 754.1 1H NMR (400 MHz, CD3OD) δ 8.16 (s, 1H), 7.24- 7.17 (m, 1H), 7.01 (t, J = 8.8 Hz, 1H), 6.21-5.98 (m, 1H), 5.41 (s, 0.59H), 5.27 (s, 0.59H), 4.78-4.70 (m, 1H), 4.33-4.16 (m, 2H), 4.08-3.97 (m, 1H), 3.91-3.80 (m, 3H), 3.72-3.60 (m, 1H), 3.51-3.37 (m, 1H), 3.17- 2.80 (m, 4H), 2.53-1.74 (m, 8H), 1.62-1.46 (m, 3H). |
| 613 | 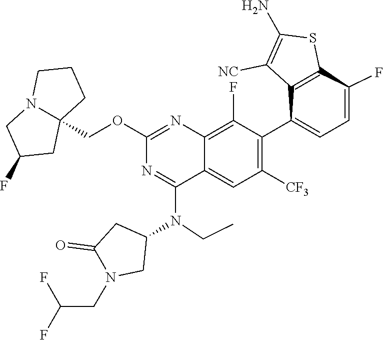 | LCMS m/z [M + 1]: 754.1 1H NMR (400 MHz, CD3OD) δ 8.17 (s, 1H), 7.26- 7.16 (m, 1H), 7.01 (t, J = 8.8 Hz, 1H), 6.25-6.03 (m, 1H), 5.42 (s, 0.60H), 5.29 (s, 0.58H), 4.80-4.73 (m, 1H), 4.35-4.20 (m, 2H), 4.08-4.01 (m, 1H), 3.91-3.81 (m, 3H), 3.72-3.61 (m, 1H), 3.45-3.37 (m, 1H), 3.19- 2.76 (m,4H), 2.58-1.75 (m, 8H), 1.57-1.46 (m, 3H). |
| 614 | 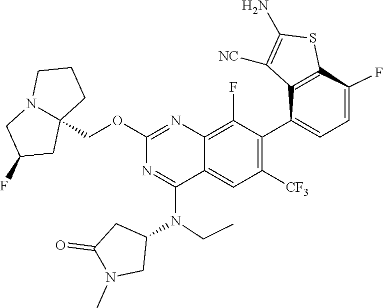 | LCMS m/z [M + 1]: 704.3 1H NMR (400 MHz, CD3OD) δ 8.16 (s, 1H), 7.23- 7.18 (m, 1H), 7.01 (m, 1H), 5.40 (s, 0.5H), 5.26 (s, 0.5H), 4.74 (m, 1H), 4.20-4.09 (m, 2H), 3.90-3.77 (m, 4H), 3.49-3.35 (m, 1H), 3.23 (m, 2H), 3.06-2.94 (m, 2H), 2.93 (s, 3H), 2.86-2.78 (m, 1H), 2.34 (m, 1H), 2.22-2.10 (m, 2H), 2.02-1.89 (m, 3H), 1.52 (m, 3H). |
| 615 | 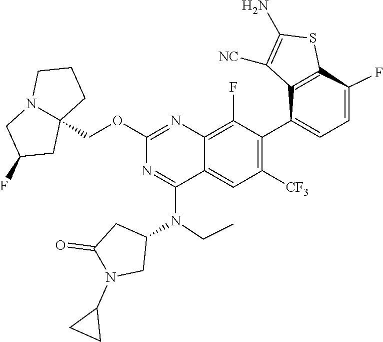 | LCMS m/z [M + 1]: 730.1 1H NMR (400 MHz, CD3OD) δ 8.14 (s, 1H), 7.20 (t, 1H), 7.00 (t, 1H), 5.39 (s, 0.5H), 5.26 (s, 0.5H), 4.65- 4.67 (m, 1H), 4.16-4.25 (m, 2H), 3.70-3.86 (m, 4H), 3.37-3.41 (m, 1H), 3.16-3.24 (m, 2H), 2.96-3.02 (m, 2H), 2.76-2.86 (m, 2H), 2.13-2.41 (m, 3H), 1.88-2.02 (m, 3H), 1.41-1.52 (m, 3H), 0.74-0.82 (m, 4H). |
| 616 | 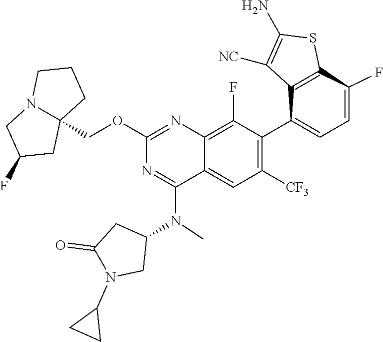 | LCMS m/z [M + 1]: 716.3 1H NMR (400 MHz, CD3OD) δ 8.26 (s, 1H), 7.20 (t, 1H), 7.00 (t, 1H), 5.38 (s, 0.5H), 5.28 (s, 0.5H), 5.25-5.27 (m, 1H), 4.29-4.32 (m, 1H), 4.21-4.24 (m, 1H), 3.88-3.94 (m, 1H), 3.60-3.66 (m, 1H), 3.44 (s, 3H), 3.34-3.36 (m, 1H), 3.19-3.22 (m, 2H), 3.00-3.04 (m, 1H), 2.90-2.94 (m, 1H), 2.72-2.83 (m, 2H), 2.13- 2.38 (m, 3H), 1.99-2.02 (m, 2H), 1.90-1.94 (m, 1H), 0.80-0.81 (m, 4H). |
| 616a | 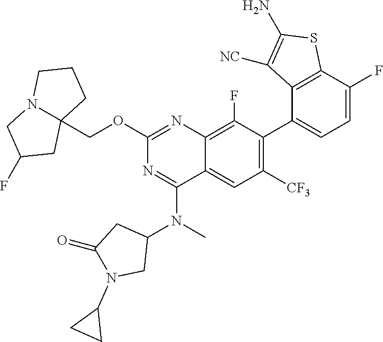 [SMILES: CN(c1nc(OCC23CCCN2CC(F)C3)nc2c(F)c(-c3ccc(F)c4sc(N)c(C#N)c34)c(C(F)(F)F)cc12)C1CC(=O)N(C2CC2)C1] | |
| 617 | 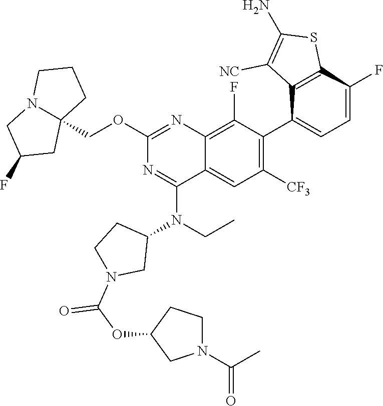 | LCMS m/z [M + 1]: 831.20 1H NMR (400 MHz, CD3OD) δ 8.25 (s, 1H), 7.23- 7.20 (m, 1H), 7.01 (t, 1H), 5.46 (s, 0.5H), 5.31 (d, 1H), 5.25 (s, 0.5H), 5.07-4.99 (m, 1H), 4.47-4.36 (m, 2H), 3.95-3.76 (m, 4H), 3.75-3.56 (m, 5H), 3.52-3.41 (m, 4H), 3.18 (s, 1H), 2.49-2.36 (m, 4H), 2.23 (d, 2H), 2.16-2.03 (m, 6H), 2.00-1.94 (m, 1H), 1.45-1.44 (m, 3H). |
| 617a | 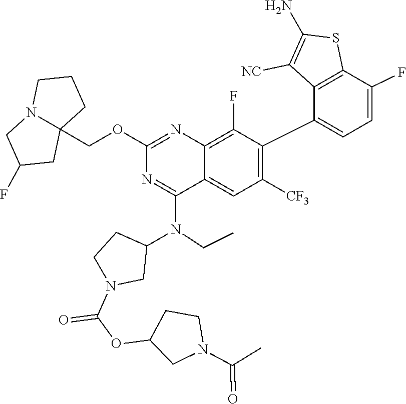 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2CCN(C(C)=O)C2)C1] | |
| 619 | 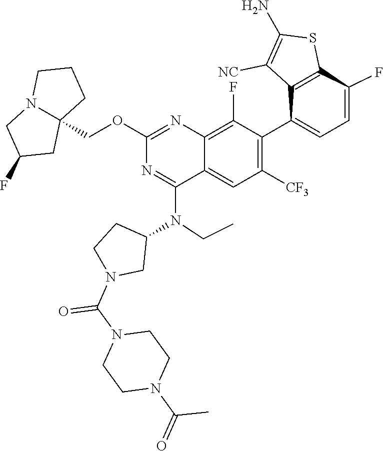 | LCMS m/z [M + 1]: 830.3 1H NMR (400 MHz, CD3OD) δ 8.39 (s, 1H), 7.28 (s, 1H), 7.02 (t, 1H), 5.54 (s, 0.5H), 5.41 (s, 0.5H), 5.06- 5.00 (m, 1H), 4.64-4.58 (m, 1H), 4.52-4.47 (m, 1H), 3.98-3.77 (m, 4H), 3.76-3.51 (m, 9H), 3.49-3.31 (m, 5H), 2.60-2.51 (m, 1H), 2.49-2.30 (m, 4H), 2.27-2.19 (m, 2H), 2.15-1.97 (m, 4H), 1.53-1.41 (m, 3H). |
| 619a | 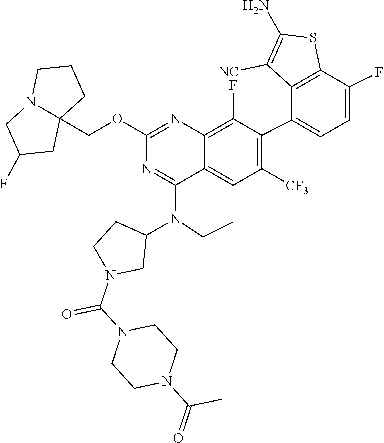 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2CCN(C(C)=O)CC2)C1] | |
| 620 | 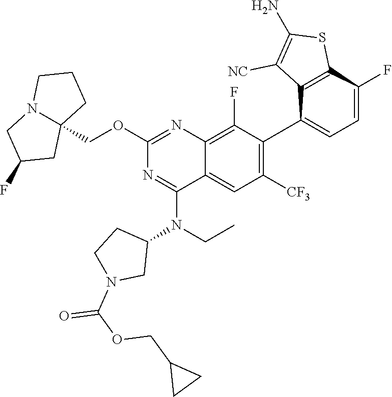 | LCMS m/z [M + 1]: 774.20 1H NMR (400 MHz, CD3OD) δ 8.25 (s, 1H), 7.21 (t, 1H), 7.01 (t, 1H), 5.48 (s, 0.5H), 5.33 (s, 0.5H), 5.04 (t, 1H), 4.48-4.37 (m, 2H), 3.95-3.86 (m, 5H), 3.76 (s, 1H), 3.67-3.59 (m, 2H), 3.53-3.44 (m, 3H), 3.19 (s, 1H), 2.52-2.32 (m, 4H), 2.24-2.22 (m, 1H), 2.12 (s, 2H), 1.99 (s, 1H), 1.46 (t, 3H), 1.15 (s, 1H), 0.55 (s, 2H), 0.30 (s, 2H). |
| 620a | 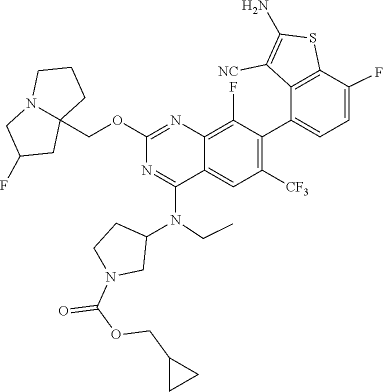 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OCC2CC2)C1] | |
| 621 | 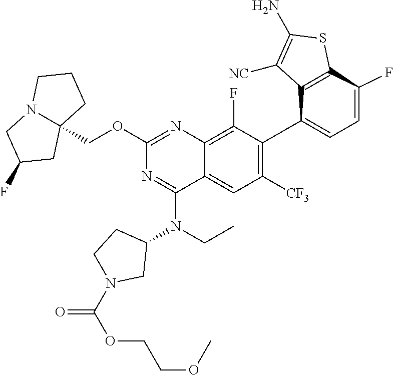 | LCMS m/z [M + 1]: 778.2 1H NMR (400 MHz, CD3OD) δ 8.22 (s, 1H), 7.19- 7.23 (m, 1H), 7.00 (t, 1H), 5.37 (s, 0.5H), 5.24 (s, 0.5H), 4.97-5.05 (m, 1H), 4.22-4.31 (m, 4H), 3.75- 3.93 (m, 4H), 3.57-3.63 (m, 3H), 3.42-3.47 (m, 1H), 3.37 (d, 3H), 3.12-3.26 (m, 3H), 2.99-3.05 (m, 1H), 2.21-2.50 (m, 4H), 2.11-2.17 (m, 1H), 1.87-2.01 (m, 3H), 1.44 (t, 3H). |
| 621a | 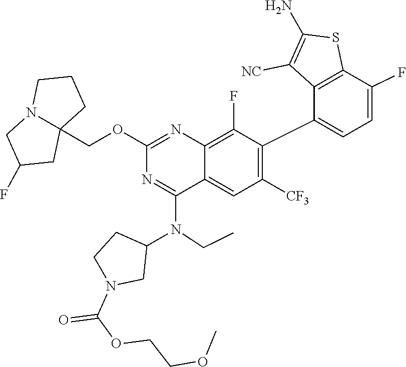 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OCCOC)C1] | |
| 622 | 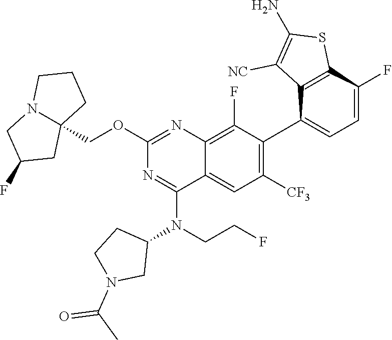 | LCMS m/z [M + 1]: 736.1 1HNMR (400 MHz, CD3OD) δ 8.21-8.19 (m, 1H), 7.23-7.20 (m, 1H), 7.03-6.99 (m, 1H) 5.36-5.04 (m, 3H), 4.83 (s, 1H), 4.78 (s, 1H), 4.66-4.28 (m, 2H), 4.28-3.85 (m, 3H), 3.85-3.79 (m, 2H), 3.79-3.66 (m, 2H), 3.66-3.60 (m, 1H), 3.30-3.18 (m, 1H), 3.18- 3.083 (m, 1H), 2.28-1.85 (m, 11H). |
| 622a | 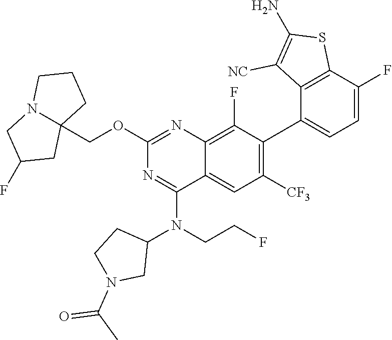 [SMILES: CC(=O)N1CCC(N(CCF)c2nc(OCC34CCCN3CC(F)C4)nc3c(F)c(-c4ccc(F)c5sc(N)c(C#N)c45)c(C(F)(F)F)cc23)C1] | |
| 623 | 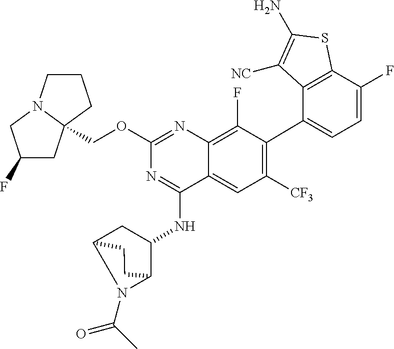 | LCMS m/z [M + 1]: 744.3 1H NMR (400 MHz, CD3OD) δ 8.51 (brs, 1H), 8.27 (s, 1H), 7.23 (t, 1 H), 7.02 (t, J = 9.2 Hz, 1 H)), 5.51- 5.33 (m, 1 H), 4.60-4.38 (m, 4H), 4.02 (s, 1H) 3.85 (m, 1H), 3.55-3.42 (m, 3H), 3.31-3.13 (m, 1H), 2.58- 2.33 (m, 4H), 2.16-2.12 (m, 8H), 1.74-1.56 (m, 4H), 1.15 (t, J = 7.6 Hz, 1 H). |
| 623a | 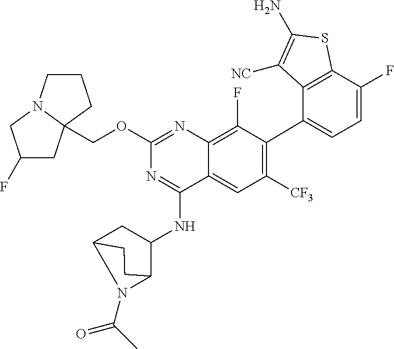 [SMILES: CC(=O)N1C2CCC1C(Nc1nc(OCC34CCCN3CC(F)C4)nc3c(F)c(-c4ccc(F)c5sc(N)c(C#N)c45)c(C(F)(F)F)cc13)C2] | |
| 624 | 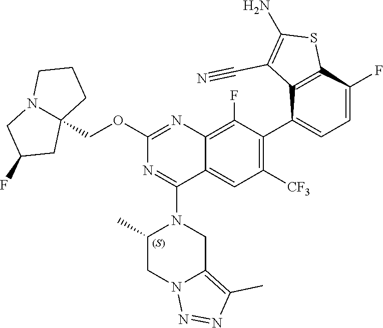 | LCMS m/z [M + 1]: 714.1 1H NMR (400 MHz, CD3OD) δ8.30 (s, 1H), 7.26- 7.22 (m, 1H), 7.03 (t, J = 8.8 Hz, 1H), 5.52-5.38 (d, 1H), 5.21-5.15 (q, 2H), 4.96 (s, 1H), 4.61-4.49 (m, 2H), 4.06-4.01 (m, 1H), 3.74-3.56 (m, 3H), 3.29 (s, 1H), 2.53-1.98 (m, 9H), 1.77 (d, J = 6.4, 3H). |
| 624a | 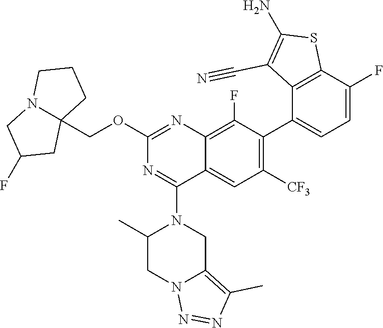 [SMILES: Cc1nnn2c1CN(c1nc(OCC34CCCN3CC(F)C4)nc3c(F)c(-c4ccc(F)c5sc(N)c(C#N)c45)c(C(F)(F)F)cc13)C(C)C2] | |
| 625 | 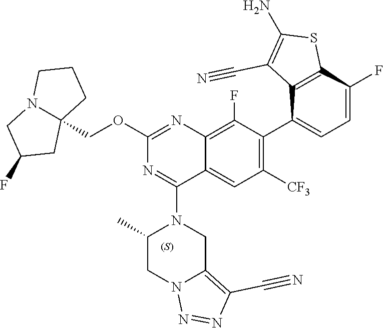 | LCMS m/z [M + 1]: 725.3 ¹H NMR (400 MHz, CD3OD) δ 8.46 (brs, 0.45H), 8.30 (s, 1H), 7.25-7.22 (m, 1H), 7.03 (t, J = 8.8 Hz, 1H), 5.57-5.26 (m, 3H), 5.12-5.02 (m, 1H), 4.67-4.40 (m, 3H), 4.16-4.00 (m, 1H), 3.78-3.46 (m, 3H), 3.27- 3.21 (m, 1H), 2.57-1.96 (m, 6H), 1.80 (d, J = 6.4 Hz, 3H). |
| 625a | 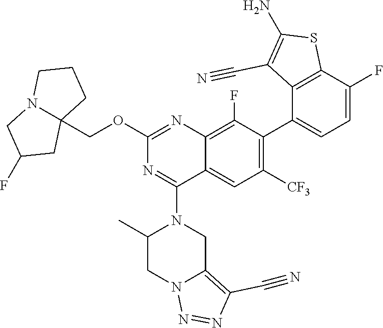 [SMILES: CC1Cn2nnc(C#N)c2CN1c1nc(OCC23CCCN2CC(F)C3)nc2c(F)c(-c3ccc(F)c4sc(N)c(C#N)c34)c(C(F)(F)F)cc12] | |
| 626 | 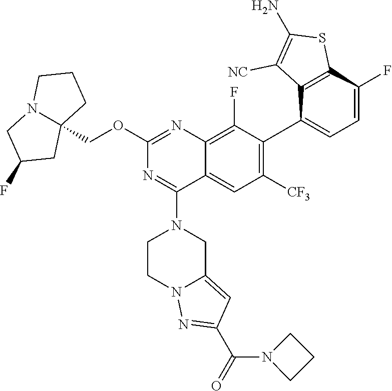 | LCMS m/z [M + 1]: 768.22 1H NMR (400 MHz, CD3OD) δ 8.27 (s, 1H), 7.26- 7.21 (m, 1H), 7.03 (t, J = 8.6 Hz, 1H), 6.64 (s, 1H), 5.49 (s, 0.5H), 5.36 (s, 0.5H), 5.32-5.22 (m, 2H), 4.65- 4.42 (m, 8H), 4.18 (t, J = 7.7 Hz, 2H), 3.69-3.50 (m, 3H), 3.27-3.19 (m, 1H), 2.55-2.28 (m, 5H), 2.21-2.12 (m, 2H), 2.08-1.96 (m, 1H). |
| 626a | 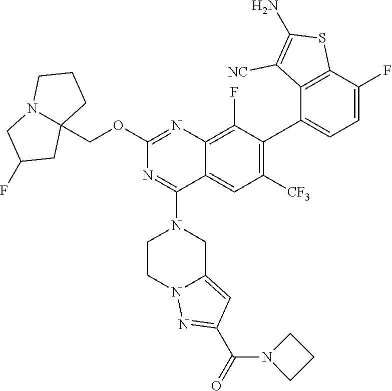 | |
| 627 | 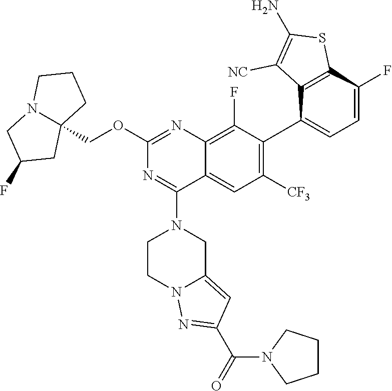 | LCMS m/z [M + 1]: 782.24 1H NMR (400 MHz, CD3OD) δ 8.23 (s, 1H), 7.25- 7.20 (m, 1H), 7.02 (t, J = 8.8 Hz, 1H), 6.66-6.60 (m, 1H), 5.37 (s, 0.5H), 5.24 (s, 2.5H), 4.56-4.25 (m, 6H), 3.92 (t, J = 6.4 Hz, 2H), 3.60 (t, J = 6.5 Hz, 2H), 3.28-3.16 (m, 3H), 3.05-2.98 (m, 1H), 2.39-2.20 (m, 2H), 2.18-2.11 (m, 1H), 2.04-1.86 (m, 7H). |
| 627a | 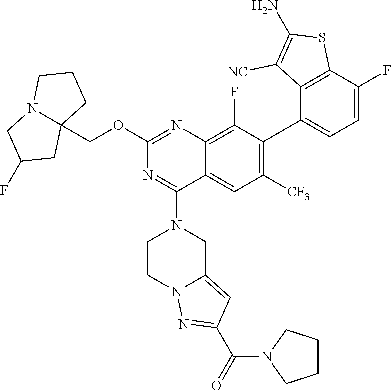 | |
| 628 | 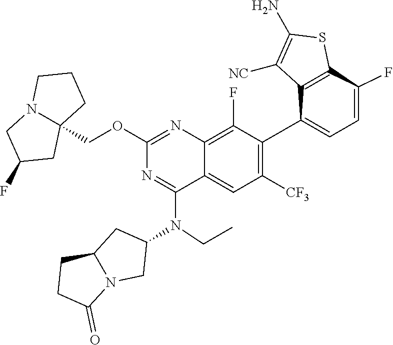 | LCMS m/z [M + 1]: 730.23 1H NMR (400 MHz, CD3OD) δ 8.16 (s, 1H), 7.24- 7.17 (m, 1H), 7.05-6.96 (m, 1H), 5.48 (s, 0.5H), 5.37 (s, 0.5H), 4.94-4.86 (m, 1H), 4.75-4.65 (m, 1H), 4.36-4.20 (m, 2H), 4.17-4.06 (m, 1H), 3.92-3.77 (m, 2H), 3.40-3.34 (m, 1H), 3.24-3.15 (m, 2H), 3.05-3.96 (m, 1H), 2.78-2.64 (m, 1H), 2.55-1.73 (m, 12H), 1.53-1.42 (m, 3H). |
| 629 | 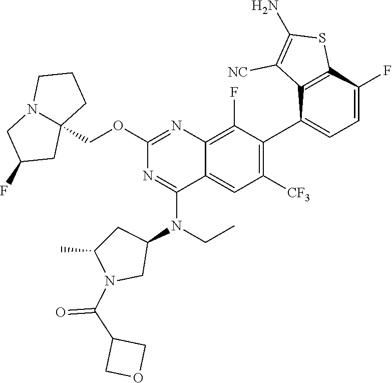 | LCMS m/z [M + 1]: 774.26. 1H NMR (400 MHz, DMSO) δ 8.14 (s, 1H), 8.07 (s, 2H), 7.29-7.22 (m, 1H), 7.14 (t, J = 9.2 Hz, 1H), 5.36 (s, 0.5H), 5.22 (s, 0.5H), 5.14-5.02 (m, 1H), 4.74-4.65 (m, 2H), 4.65-4.57 (m, 1H), 4.36-4.25 (m, 1H), 4.19- 3.96 (m, 3H), 3.87-3.78 (m, 1H), 3.72-3.56 (m, 2H), 3.52-3.42 (m, 1H), 3.18-2.97 (m, 3H), 2.87-2.76 (m, 1H), 2.22-1.94 (m, 5H), 1.91-1.68 (m, 4H), 1.33-1.27 (m, 2H), 1.26-1.15 (m, 3H), 1.15-1.10 (m, 1H). |
| 630 | 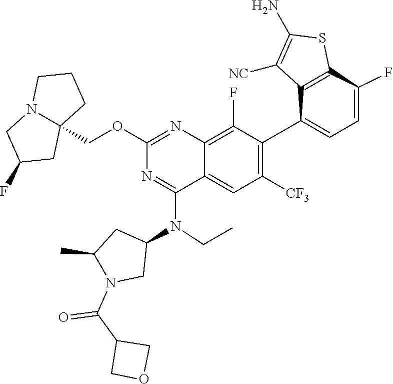 | LCMS m/z [M + 1]: 774.3 1H NMR (400 MHz, CD3OD) δ 8.28 (d, 1H), 7.23 (m, 1H), 7.03 (m, 1H), 6.81-6.88 (m, 0.5H), 6.60- 6.68 (m, 0.5H), 6.33 (d, 1H), 5.98-6.06 (m, 1H), 5.50-5.83 (m, 1H), 5.40 (s, 0.5H), 5.27 (s, 0.5H), 4.50-4.60 (m, 1H), 3.84-3.92 (m, 1H), 3.67-3.78 (m, 1H), 3.59-3.67 (m, 1H), 3.40 (d, 3H), 3.10-3.29 (m, 3H), 2.52-2.68 (m, 2H), 2.10-2.42 (m, 4H), 1.91-2.08 (m, 3H). |
| 631 | 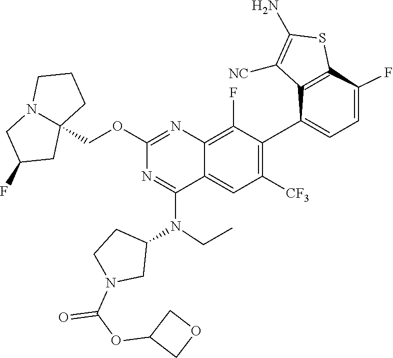 | LCMS m/z [M + 1]: 776.3 1H NMR (400MHz, CD3OD) δ 8.26 (s, 1H), 7.27- 7.16 (m, 1H), 7.06-6.89 (m, 1H), 5.49 (s, 0.52H), 5.44-5.37 (m, 1H), 5.35 (s, 0.49H), 5.12-5.00 (m, 1H), 4.91-4.87 (m, 2H), 4.71-4.57 (m, 2H), 4.55-4.33 (m, 2H), 4.06-3.72 (m, 4H), 3.67-3.41 (m, 5H), 3.23 (s, 1H), 2.53-1.95 (m, 8H), 1.52-1.39 (m, 3H). |
| 631a | 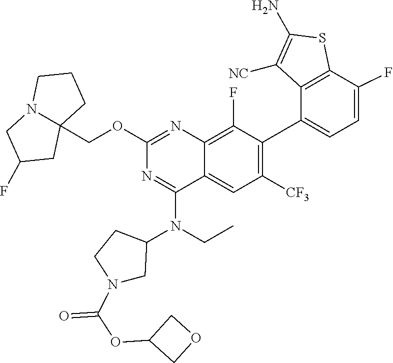 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2COC2)C1] | |
| 632 | 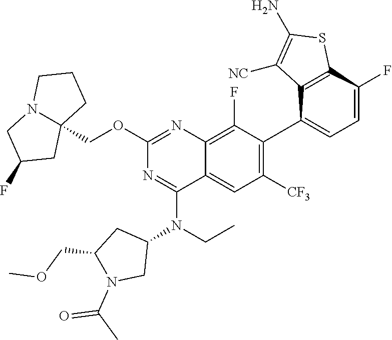 | LCMS m/z [M + 1]: 762.3 1H NMR (400 MHz, CD3OD) δ 8.28 (d, 1H), 7.23 (m, 1H), 7.03 (m, 1H), 6.81-6.88 (m, 0.5H), 6.60- 6.68 (m, 0.5H), 6.33 (d, 1H), 5.98-6.06 (m, 1H), 5.50-5.83 (m, 1H), 5.40 (s, 0.5H), 5.27 (s, 0.5H), 4.50-4.60 (m, 1H), 3.84-3.92 (m, 1H), 3.67-3.78 (m, 1H), 3.59-3.67 (m, 1H), 3.40 (d, 3H), 3.10-3.29 (m, 3H), 2.52-2.68 (m, 2H), 2.10-2.42 (m, 4H), 1.91-2.08 (m, 3H). |
| 632a | 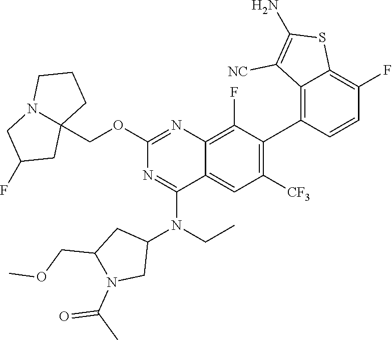 | |
| 633 | 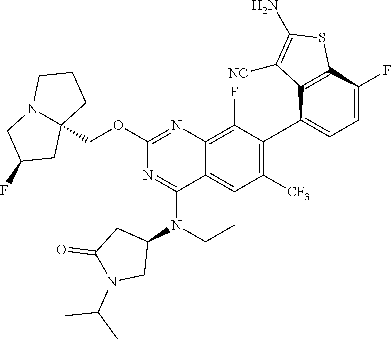 | |
| 633a | 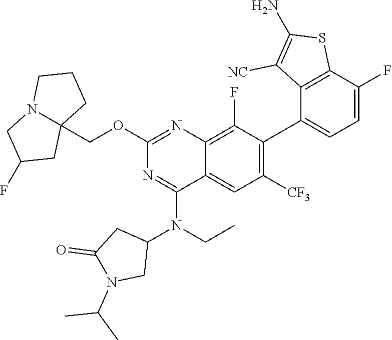 [SMILES: CCN(c1nc(OCC23CCCN2CC(F)C3)nc2c(F)c(-c3ccc(F)c4sc(N)c(C#N)c34)c(C(F)(F)F)cc12)C1CC(=O)N(C(C)C)C1] | |
| 634 | 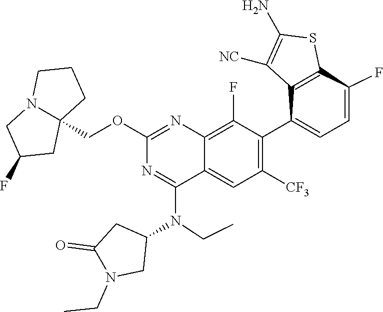 | LCMS m/z [M + 1]: 718.4 1H NMR (400 MHz, CD3OD) δ 8.16 (s, 1H), 7.24- 7.18 (m, 1H), 7.00 (t, 1H), 5.39 (m, 0.5H), 5.26 (m, 0.5H), 4.82-4.77 (m, 1H), 4.24-4.16 (m, 2H), 3.97- 3.91 (m, 1H), 3.89-3.81 (m, 2H), 3.79-3.74 (m, 1H), 3.59-3.52 (m, 1H), 3.28-3.21 (m, 2H), 3.05-2.97 (m, 2H), 2.86-2.79 (m, 1H), 2.41-1.86 (m, 8H), 1.51 (t, 3H), 1.19 (t, 3H). |
| 635 | 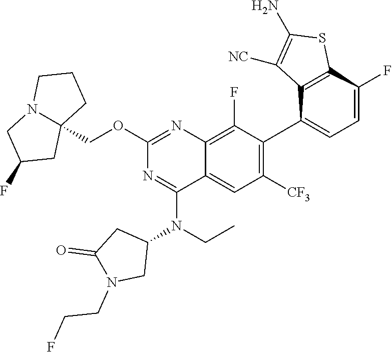 | LCMS m/z [M + 1]: 736.10 1H NMR (400 MHz, CD3OD) δ 8.20 (s, 1H), 7.23- 7.20 (m, 1H), 7.02 (t, 1H), 5.60-5.58 (m,0.5H), 5.47- 5.45 (m, 0.5H), 4.79-4.67 (m, 2H), 4.59-4.40 (m, 3H), 4.03 (t, 1H), 3.97-3.77 (m, 6H), 3.73-3.60 (m, 2H), 3.41-3.35 (m, 1H), 3.08-2.97 (m, 1H), 2.92-2.85 (m, 1H), 2.57-2.22 (m,5H), 2.08 (m, 1H), 1.57 (t, 3H). |
| 635a | 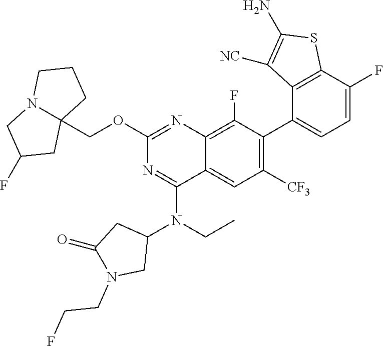 [SMILES: CCN(c1nc(OCC23CCCN2CC(F)C3)nc2c(F)c(-c3ccc(F)c4sc(N)c(C#N)c34)c(C(F)(F)F)cc12)C1CC(=O)N(CCF)C1] | |
| 636 | 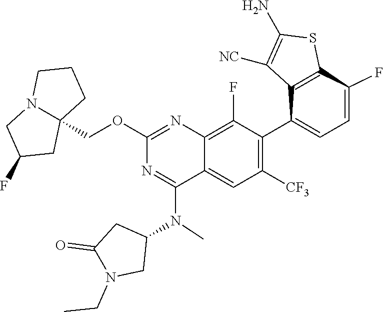 | LCMS m/z [M + 1]: 704.1 1H NMR (400 MHz, CD3OD) δ 8.26 (s, 1H), 7.24- 7.17 (m, 1H), 7.00 (t, 1H), 5.40-5.29 (m, 1.5H), 5.23 (s, 0.5H), 4.32-4.19 (m, 2H), 4.04-3.96 (m, 1H), 3.74-3.66 (m, 1H), 3.47-3.35 (m, 5H), 3.28-3.11 (m, 3H), 3.05-2.90 (m, 2H), 2.85-2.76 (m, 1H), 2.36-2.11 (m, 3H), 2.03-1.86 (m, 3H), 1.23-1.16 (m, 3H). |
| 636a | 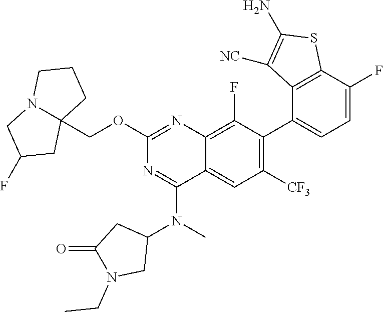 [SMILES: CCN1CC(N(C)c2nc(OCC34CCCN3CC(F)C4)nc3c(F)c(-c4ccc(F)c5sc(N)c(C#N)c45)c(C(F)(F)F)cc23)CC1=O] | |
| 637 | 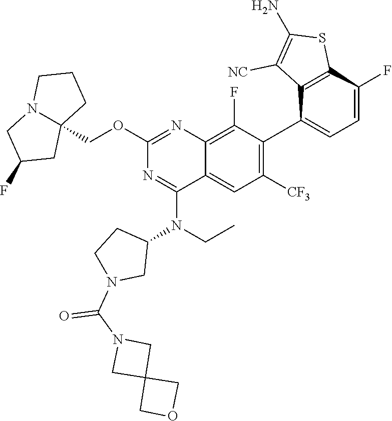 | LCMS m/z [M + 1]: 801.2 1H NMR (400 MHz, CD3OD) δ 8.23 (s, 1H), 7.24- 7.18 (m, 1H), 7.00 (t, 1H), 5.37 (s, 0.5H), 5.24 (s, 0.5H), 5.01-4.95 (m, 1H), 4.80-4.73 (m, 4H), 4.32- 4.27 (m, 1H), 4.26-4.21 (m, 2H), 4.18-4.10 (m, 3H), 3.88-3.82 (m, 2H), 3.71-3.65 (m, 1H), 3.59-3.53 (m, 1H), 3.49-3.38 (m, 2H), 3.26-3.20 (m, 2H), 3.05-2.99 (m, 1H), 2.65-2.57 (m, 1H), 2.40-2.28 (m, 3H), 2.22- 2.09 (m, 2H), 2.05-1.89 (m, 3H), 1.48-1.36 (m, 3H). |
| 637a | 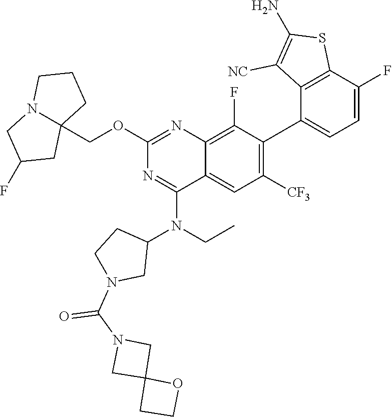 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2CC3(CCO3)C2)C1] | |
| 638 | 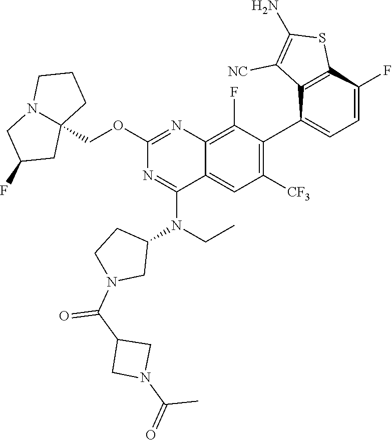 | LCMS m/z [M + 1]: 801.2 1H NMR (400 MHz, CD3OD) δ 8.24 (s, 1H), 7.20- 7.23 (m, 1H), 7.01 (t, 1H), 5.39 (s, 0.5H), 5.25 (s, 0.5H), 4.98-5.09 (m, 1H), 4.33-4.41 (m, 3H), 3.80- 4.29 (m, 7H), 3.47-3.77 (m, 4H), 3.23-3.25 (m, 2H), 3.02-3.09 (m, 1H), 2.13-2.53 (m, 5H), 1.93-2.05 (m, 3H), 1.87 (d, 3H), 1.43-1.48 (m, 3H). |
| 638a | 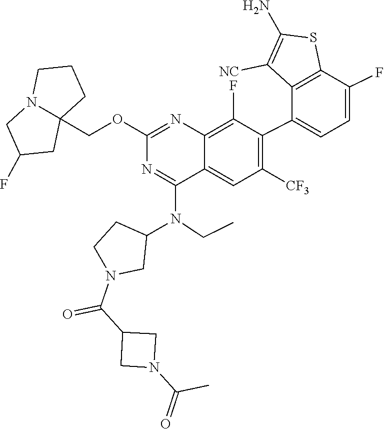 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CN(C(C)=O)C2)C1] | |
| 639 | 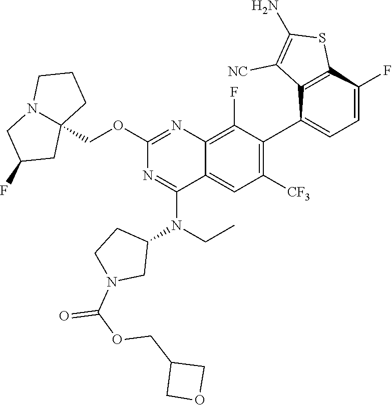 | LCMS m/z [M + 1]: 790.2 1H NMR (400 MHz, CD3OD) δ 8.22 (s, 1H), 7.19- 7.23 (m, 1H), 7.01 (t, 1H), 5.37 (s, 0.5H), 5.23 (s, 0.5H), 4.99-5.06 (m, 1H), 4.78-4.82 (m, 2H), 4.50- 4.55 (m, 2H), 4.21-4.31 (m, 4H), 3.74-3.96 (m, 4H), 3.38-3.63 (m, 3H), 3.12-3.24 (m, 3H), 2.98-3.04 (m, 1H), 2.17-2.47 (m, 4H), 2.10-2.12 (m, 1H), 1.86- 2.02 (m, 3H), 1.44 (t, 3H). |
| 639a | 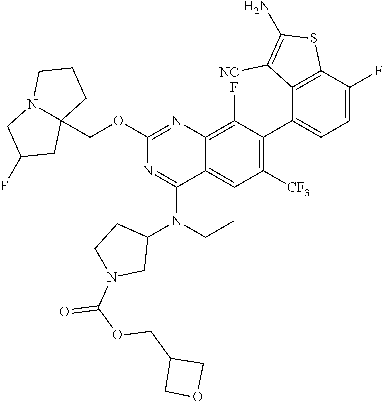 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OCC2COC2)C1] | |
| 640 | 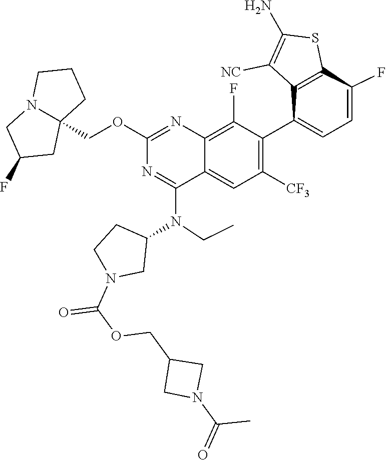 | LCMS m/z [M + 1]: 831.30 1H NMR (400 MHz, CD3OD) δ 8.23 (s, 1H), 7.23- 7.20 (m, 1H), 7.01 (t, 1H), 5.39 (s, 0.5H), 5.26 (s, 0.5H), 5.02 (t, 1H), 4.37-4.21 (m, 5H), 4.10-4.00 (m, 2H), 3.95-3.63 (m, 6H), 3.57 (t, 1H), 3.49-3.39 (m, 2H), 3.00 (d, 3H), 2.47-2.11 (m, 6H), 2.06-1.98 (m, 2H), 1.88-1.77 (m, 3H), 1.44 (t, 3H). |
| 640a | 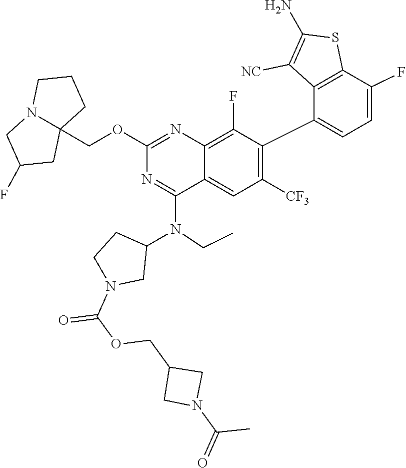 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OCC2CN(C(C)=O)C2)C1] | |
| 641 | 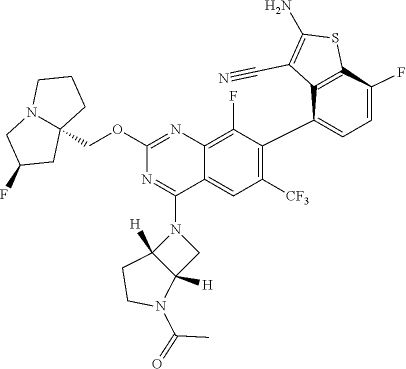 | LCMS m/z [M + 1]: 702.20 1H NMR (400 MHz, CD3OD) δ 8.51 (brs, 1H), 8.07 (s, 1H), 7.20 (s, 1H), 7.01 (t, J = 8.8 Hz, 1 H), 5.71 (d, J = 117.6 Hz, 1 H), 5.40 (d, J = 93.6 Hz, 1 H), 4.93 (s, 1H), 4.45-4.22 (m, 3H), 4.08-3.90 (m, 1H), 3.59-3.48 (m, 4H), 3.19 (s, 1H), 2.67-2.37 (m, 5H), 2.18 (s, 2H), 2.14 (s, 3H), 1.97 (s, 1 H). |
| 642 | 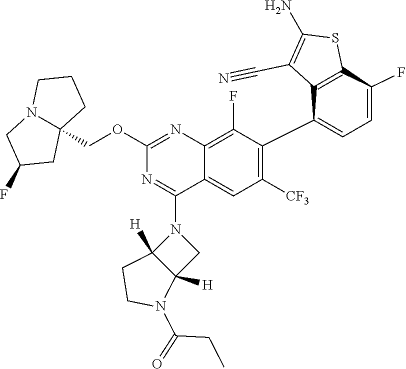 | LCMS m/z [M + 1]: 716.3 1H NMR (400 MHz, CD3OD) δ 8.05 (s, 1H), 7.19 (s, 1H), 7.20 (s, 1H), 7.00 (t, J = 9.2 Hz, 1 H), 5.60 (d, J = 45.2 Hz, 1 H), 5.31 (d, J = 53.6 Hz, 1 H), 4.94 (s, 1H), 4.57-4.43 (m, 1H), 4.33-4.24 (m, 2H), 4.06 (t, J = 9.6 Hz, 1 H), 3.90-3.86 (m, 1H), 3.69-3.53 (m, 1H), 3.41-3.32 (m, 1H), 3.27-3.17 (m, 2H), 3.09-2.99 (m, 1H), 2.69-2.43 (m, 3H), 2.40-2.14 (m, 4H), 2.07- 1.86 (m, 3H), 1.21-1.07 (m, 3H). |
| 643 | 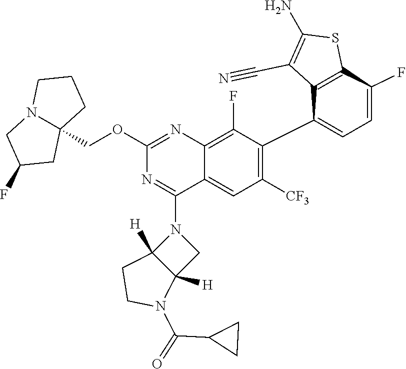 | LCMS m/z [M + 1]: 728.1 1HNMR (400 MHz, CD3OD) δ 8.52 (s, 0.5 H), 8.02- 8.12 (m, 1 H), 7.15-7.23 (m, 1 H), 6.96-7.04 (m, 1 H), 5.52-5.71 (m, 1 H), 5.41 (s, 0.5 H), 5.27 (s, 0.5 H), 5.16 (s, 0.5 H), 5.02 (s, 0.5 H), 4.88-4.96 (m, 1 H), 4.53-4.66 (m, 0.5 H), 4.19-4.43 (m, 3.5 H), 3.92- 4.07 (m, 1H), 3.61-3.69 (m, 0.5 H), 3.33-3.49 (m, 2.5 H), 3.03-3.15 (m, 1 H), 2.54-2.73 (m, 1 H), 1.88-2.41 (m, 8 H), 0.80-1.01 (m, 4 H). |
| 644 | 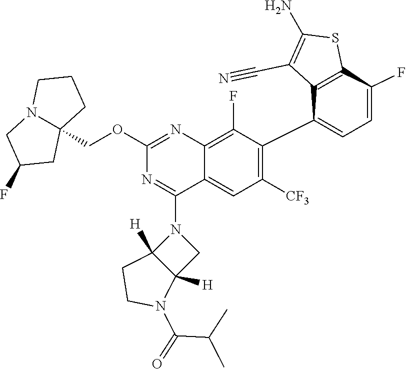 | LCMS m/z [M + 1]: 760.2 1H NMR (400 MHz, CD3OD) δ 8.48 (brs, 1H), 8.07 (s, 1H), 7.23-7.16 (m, 1H), 7.00 (t, J = 8.8 Hz, 1H), 5.75-5.54 (m, 1H), 5.49-5.35 (m, 1H), 5.04-4.93 (m, 1H), 4.55-4.10 (m, 4H), 3.99-3.39 (m, 5H), 3.26-3.17 (m, 1H), 2.94-2.49 (m, 3H), 2.45-1.98 (m, 6H), 1.24- 1.05 (m, 6H). |
| 644a | 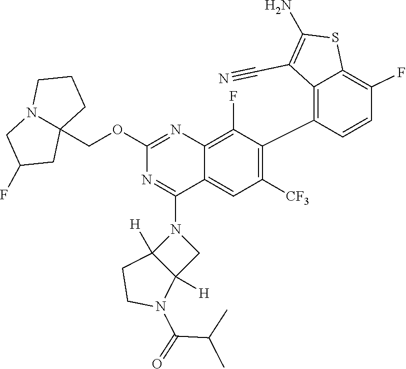 [SMILES: CC(C)C(=O)N1CCC2C1CN2c1nc(OCC23CCCN2CC(F)C3)nc2c(F)c(-c3ccc(F)c4sc(N)c(C#N)c34)c(C(F)(F)F)cc12] | |
| 645 | 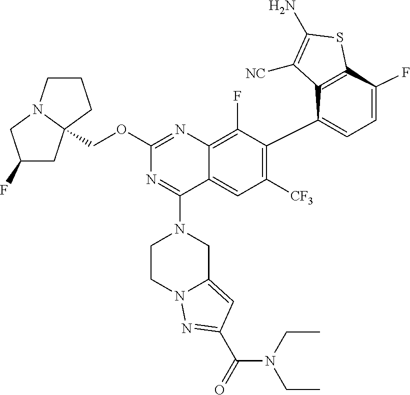 | LCMS m/z [M + 1]: 784.25 1H NMR (400 MHz, CD3OD) δ 8.28 (s, 1H), 7.32- 7.24 (m, 1H), 7.06 (t, J = 8.9 Hz, 1H), 6.56 (s, 1H), 5.41 (s, 0.5H), 5.29 (s, 2.5H), 4.62-4.29 (m, 6H), 3.82-3.68 (m, 2H), 3.60-3.51 (m, 2H), 3.32-3.15 (m, 3H), 3.09-3.01 (m,1H), 3.43-2.15 (m, 3H), 2.08-1.89 (m, 3H), 1.34-1.19 (m, 6H). |
| 645a | 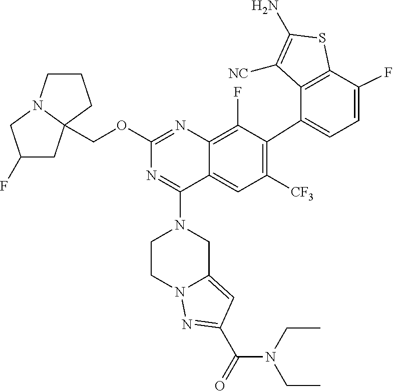 [SMILES: CCN(CC)C(=O)c1cc2n(n1)CCN(c1nc(OCC34CCCN3CC(F)C4)nc3c(F)c(-c4ccc(F)c5sc(N)c(C#N)c45)c(C(F)(F)F)cc13)C2] | |
| 646 | 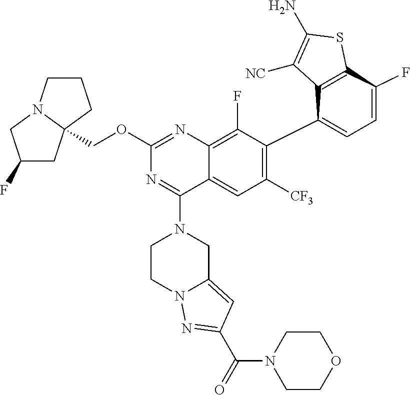 | LCMS m/z [M + 1]: 798.23. 1H NMR (400 MHz, CD3OD) δ 8.28 (s, 1H), 7.32- 7.24 (m, 1H), 7.06 (t, J = 8.9 Hz, 1H), 6.56 (s, 1H), 5.41 (s, 0.5H), 5.29 (s, 2.5H), 4.62-4.29 (m, 6H), 3.82-3.68 (m, 2H), 3.60-3.51 (m, 2H), 3.32-3.15 (m, 3H), 3.09-3.01 (m,1H), 3.43-2.15 (m, 3H), 2.08-1.89 (m, 3H), 1.34-1.19 (m, 6H). |
| 646a | 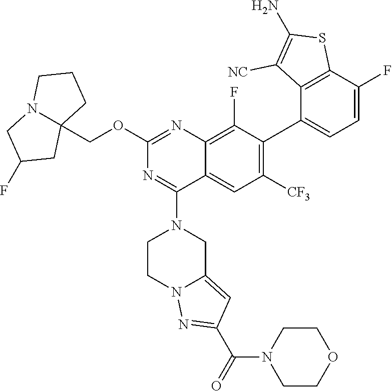 | |
| 647 | 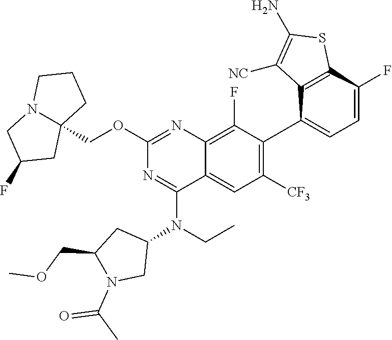 | LCMS m/z [M + 1]: 762.3 1H NMR (400 MHz, CD3OD) δ 8.22-8.19 (d, J = 12 Hz, 1H), 7.29-7.13 (m, 1H), 7.07-6.93 (m, 1H), 5.42- 5.20 (m, 2H), 4.47-4.33 (m, 1H), 4.32-4.18 (m, 2H), 4.00-3.70 (m, 4H), 3.69-3.52 (m, 1H), 3.50-3.41 (m, 1H), 3.36 (s, 3H), 3.28-3.10 (m, 3H), 3.07-2.94 (m, 1H), 2.77-2.57 (m, 1H), 2.37-2.07 (m, 7H), 2.02-1.85 (m, 3H), 1.47-1.38 (m, 3H). |
| 648 | 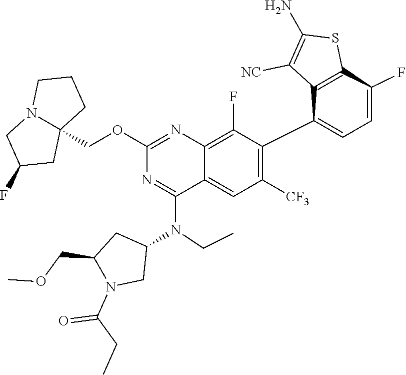 | LCMS m/z [M + 1]: 776.3 1H NMR (400 MHz, CD3OD) δ 8.22-8.20 (d, J = 8 Hz, 1H), 7.31-7.15 (m, 1H), 7.12-6.86 (m, 1H), 5.43- 5.22 (m, 2H), 4.52-4.35 (m, 1H), 4.34-4.07 (m, 2H), 3.98-3.68 (m, 4H), 3.68-3.49 (m, 1H), 3.49-3.41 (m, 1H), 3.37-3.31 (m, 3H), 3.28-3.09 (m, 3H), 3.07-2.93 (m, 1H), 2.69-2.09 (m, 7H), 2.02-1.83 (m, 3H), 1.45- 1.37 (m, 3H), 1.18-1.09 (m, 3H). |
| 649 | 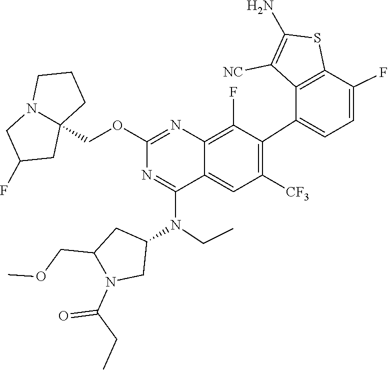 [SMILES: CCC(=O)N1C[C@@H](N(CC)c2nc(OC[C@@]34CCCN3CC(F)C4)nc3c(F)c(-c4ccc(F)c5sc(N)c(C#N)c45)c(C(F)(F)F)cc23)CC1COC] | LCMS m/z [M + 1]: 776.3 1H NMR (400 MHz, CD3OD) δ 8.28 (d, 1H), 7.23 (m, 1H), 7.03 (m, 1H), 6.81-6.88 (m, 0.5H), 6.60- 6.68 (m, 0.5H), 6.33 (d, 1H), 5.98-6.06 (m, 1H), 5.50-5.83 (m, 1H), 5.40 (s, 0.5H), 5.27 (s, 0.5H), 4.50-4.60 (m, 1H), 3.84-3.92 (m, 1H), 3.67-3.78 (m, 1H), 3.59-3.67 (m, 1H), 3.40 (d, 3H), 3.10-3.29 (m, 3H), 2.52-2.68 (m, 2H), 2.10-2.42 (m, 4H), 1.91-2.08 (m, 3H). |
| 649a | 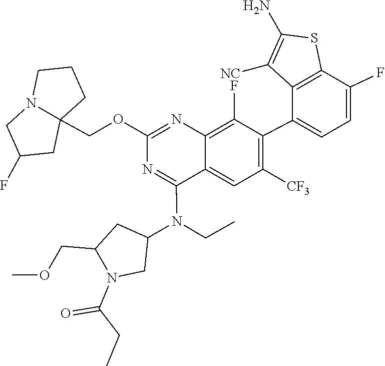 [SMILES: CCC(=O)N1CC(N(CC)c2nc(OCC34CCCN3CC(F)C4)nc3c(F)c(-c4ccc(F)c5sc(N)c(C#N)c45)c(C(F)(F)F)cc23)CC1COC] | |
| 650 | 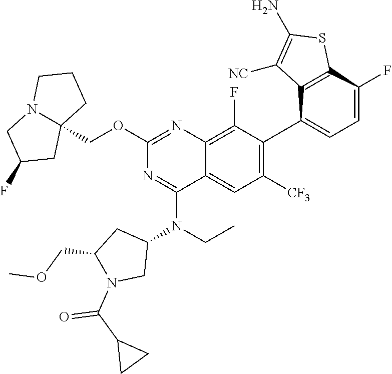 | LCMS m/z [M + 1]: 788.3 1H NMR (400 MHz, CD3OD) δ 8.28 (d, 1H), 7.23 (m, 1H), 7.03 (m, 1H), 6.81-6.88 (m, 0.5H), 6.60- 6.68 (m, 0.5H), 6.33 (d, 1H), 5.98-6.06 (m, 1H), 5.50-5.83 (m, 1H), 5.40 (s, 0.5H), 5.27 (s, 0.5H), 4.50-4.60 (m, 1H), 3.84-3.92 (m, 1H), 3.67-3.78 (m, 1H), 3.59-3.67 (m, 1H), 3.40 (d, 3H), 3.10-3.29 (m, 3H), 2.52-2.68 (m, 2H), 2.10-2.42 (m, 4H), 1.91-2.08 (m, 3H). |
| 650a | 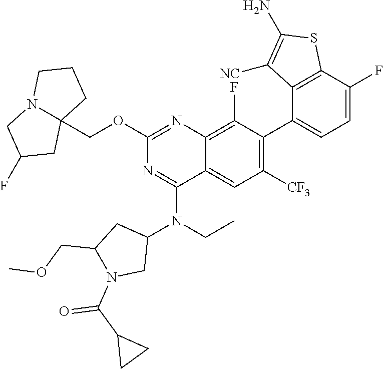 [SMILES: CCN(c1nc(OCC23CCCN2CC(F)C3)nc2c(F)c(-c3ccc(F)c4sc(N)c(C#N)c34)c(C(F)(F)F)cc12)C1CC(COC)N(C(=O)C2CC2)C1] | |
| 651 | 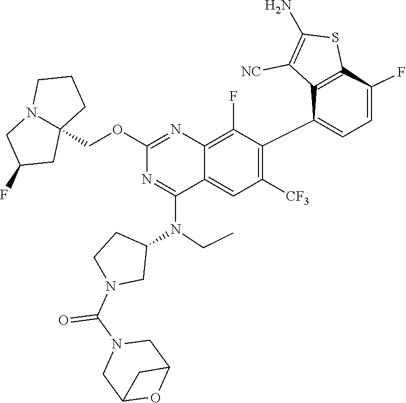 | LCMS m/z [M + 1]: 801.2 ¹H NMR (400 MHz, CD3OD) δ 8.25 (s, 1H), 7.24- 7.18 (m, 1H), 7.01 (t, 1H), 5.38 (s, 0.5H), 5.24 (s, 0.5H), 5.04-4.98 (m, 1H), 4.58 (s, 3H), 4.37-4.30 (m, 1H), 4.29-4.23 (m, 1H), 4.04-3.98 (m, 1H), 3.96-3.76 (m, 4H), 3.71-3.66 (m, 2H), 3.63-3.54 (m, 2H), 3.24- 3.11 (m, 3H), 3.07-3.01 (m, 1H), 2.50-2.30 (m, 3H), 2.30-2.17 (m, 2H), 2.17-2.11 (m, 1H), 2.05-1.96 (m, 2H), 1.94-1.84 (m, 2H), 1.46 (t, 3H). |
| 651a | 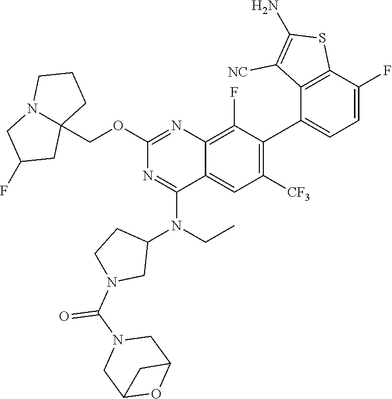 | |
| 652 | 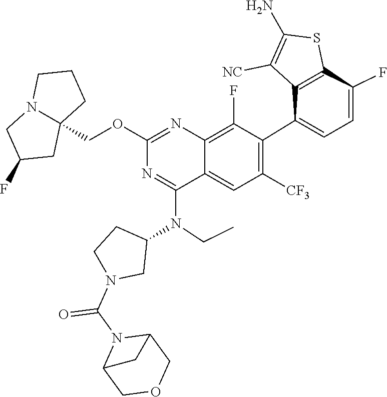 | LCMS m/z [M + 1]: 801.5 ¹H NMR (400 MHz, CD3OD) δ 8.24 (s, 1H), 7.19- 7.23 (m, 1H), 7.01 (t, 1H), 5.38 (s, 0.5H), 5.25 (s, 0.5H), 4.95-5.02 (m, 1H), 4.59 (s, 0.5H), 4.18-4.37 (m, 5.5H), 3.71-3.87 (m, 6H), 3.57 (t, 1H), 3.43- 3.48 (m, 1H), 3.21-3.30 (m, 3H), 3.02-3.03 (m, 1H), 2.64-2.69 (m, 1H), 2.45-2.50 (m, 1H), 2.12-2.38 (m, 4H), 1.90-2.02 (m, 3H), 1.78 (d, 1H), 1.46 (t, 3H). |
| 652a | 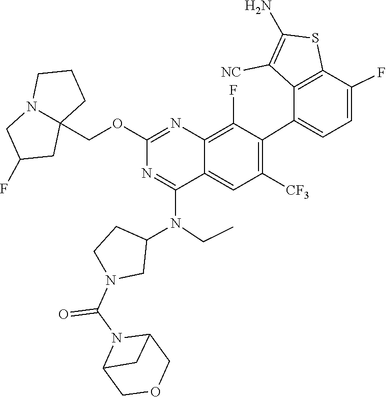 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2C3COCC2C3)C1] | |
| 653 | 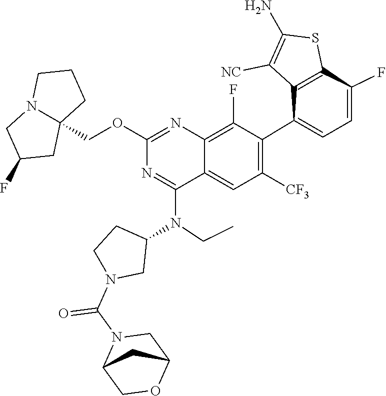 | LCMS m/z [M + 1]: 801.2 ¹H NMR (400 MHz, CD3OD) δ 8.25 (s, 1H), 7.23- 7.20 (m, 1H), 7.01 (t, 1H), 5.38 (s, 0.5H), 5.25 (s, 0.5H), 5.03-4.95 (m, 1H), 4.58 (s, 1H), 4.43 (s, 1H), 4.35-4.23 (m, 2H), 4.02 (d, 1H), 3.94-3.82 (m, 2H), 3.81-3.73 (m, 2H), 3.69-3.63 (m, 1H), 3.60-3.53 (m, 3H), 3.39-3.32 (m, 1H), 3.27-3.18 (m, 3H), 3.09-2.98 (s, 1H), 2.51-1.86 (m, 10H), 1.47 (t, 3H). |
| 653a | 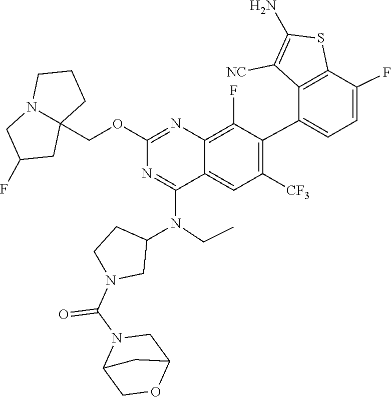 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2CC3CC2CO3)C1] | |
| 654 | 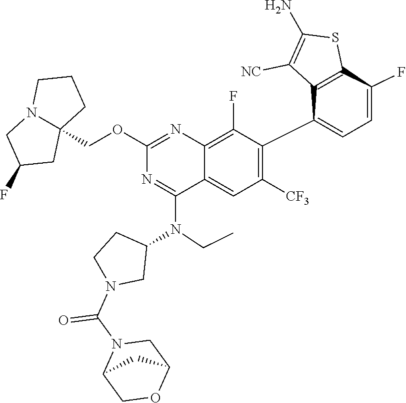 | LCMS m/z [M + 1]: 801.5 ¹H NMR (400 MHz, CD3OD) δ 8.23 (s, 1H), 7.19- 7.23 (m, 1H), 7.01 (t, 1H), 5.37 (s, 0.5H), 5.24 (s, 0.5H), 4.97-5.03 (m, 1H), 4.57 (d, 2H), 4.31 (d, 1H), 4.23 (d, 1H), 3.78-3.99 (m, 6H), 3.54-3.59 (m, 1H), 3.39-3.46 (m, 3H), 3.22-3.25 (m, 3H), 3.01-3.02 (m, 1H), 2.27-2.46 (m, 4H), 2.12-2.14 (m, 1H), 1.81-2.03 (m, 5H), 1.44 (t, 3H). |
| 655 | 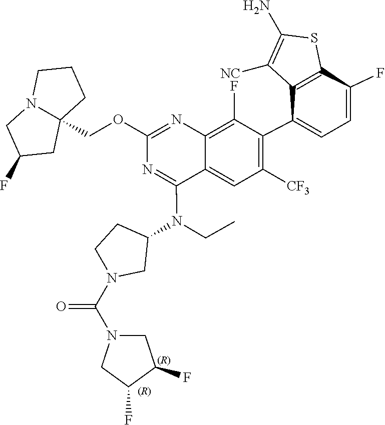 | LCMS m/z [M + 1]: 809.2 ¹H NMR (400 MHz, CD3OD) δ 8.28 (s, 1H), 7.20- 7.24 (m, 1H), 7.01 (t, 1H), 5.45 (s, 0.5H), 5.28-5.32 (m, 1.5H), 5.15-5.18 (m, 1H), 4.96-5.05 (m, 1H), 4.35-4.47 (m, 2H), 3.79-3.96 (m, 5H), 3.56-3.73 (m, 5H), 3.40-3.49 (m, 3H), 3.12-3.20 (m, 1H), 2.33-2.51 (m, 4H), 2.07-2.24 (m, 3H), 1.95-2.03 (m, 1H), 1.49 (t, 3H). |
| 656 | 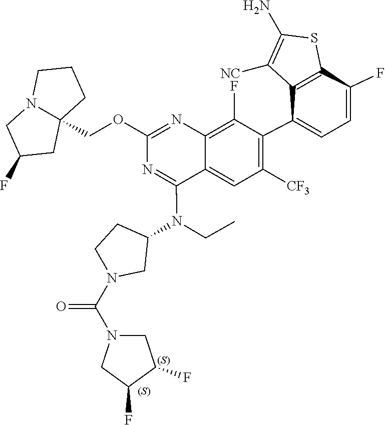 | LCMS m/z [M + 1]: 809.2 ¹H NMR (400 MHz, CD3OD) δ 8.27 (s, 1H), 7.24- 7.19 (m, 1H), 7.01 (t, 1H), 5.45 (s, 0.5H), 5.34-5.26 (m, 1.5H), 5.18-5.13 (m, 1H), 5.06-5.00 (m, 1H), 4.46-4.34 (m, 2H), 4.09-4.02 (m, 1H), 3.96-3.69 (m, 7H), 3.64-3.41 (m, 5H), 3.18-3.12 (m, 1H), 2.51-2.31 (m, 4H), 2.26-2.20 (m, 1H), 2.12-1.94 (m, 3H), 1.51- 1.41 (m, 3H). |
| 656a | 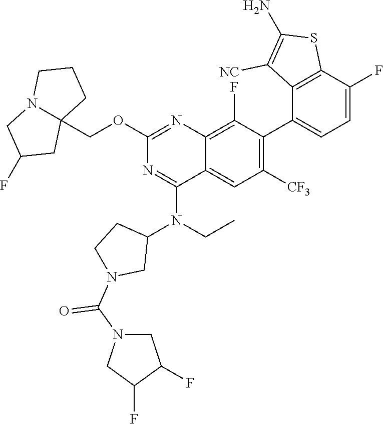 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2CC(F)C(F)C2)C1] | |
| 657 | 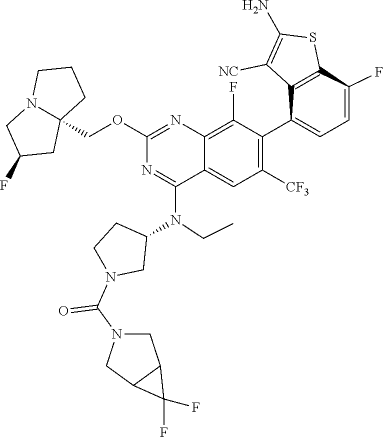 | LCMS m/z [M + 1]: 821.2 ¹H NMR (400 MHz, CD3OD) δ 8.24 (s, 1H), 7.24- 7.18 (m, 1H), 7.01 (t, 1H), 5.40-5.36 (m, 0.5H), 5.28- 5.23 (m, 0.5H), 5.01-4.94 (m, 1H), 4.35-4.23 (m, 2H), 4.11 (d, 1H), 3.95-3.74 (m, 5H), 3.66-3.55 (m, 3H), 3.54-3.48 (m, 1H), 3.39-3.48 (m, 1H), 3.27-3.22 (m, 2H), 3.08-3.01 (m, 1H), 2.46-2.21 (m, 6H), 2.17- 2.11 (m, 1H), 2.05-1.97 (m, 2H), 1.93-1.87 (m, 1H), 1.45 (t, 3H). |
| 657a | 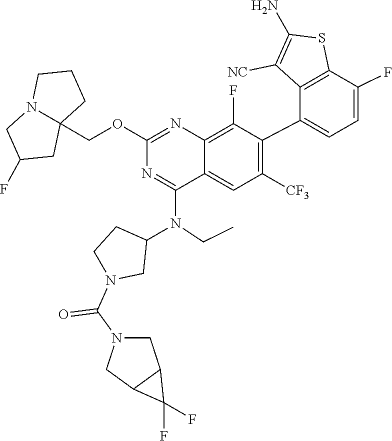 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2CC3C(C2)C3(F)F)C1] | |
| 658 | 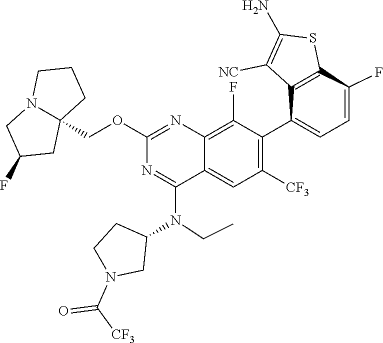 | |
| 658a | 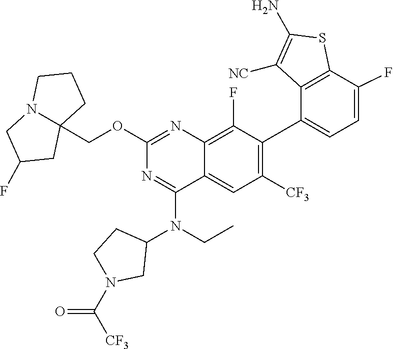 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C(F)(F)F)C1] | |
| 659 | 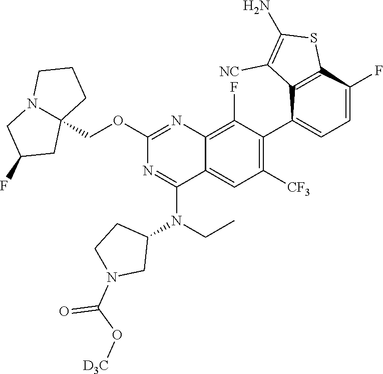 | LCMS m/z [M + 1]: 737.1 1H NMR (400 MHz, CD3OD) δ 8.22 (s, 1H), 7.19- 7.23 (m, 1H), 7.00 (t, 1H), 5.37 (s, 0.5H), 5.24 (s, 0.5H), 5.00 (s, 1H), 4.23-4.31 (m, 2H), 3.84-3.93 (m, 3H), 3.72-3.77 (m, 1H), 3.55-3.60 (m, 1H), 3.42-3.47 (m, 1H), 3.21-3.26 (m, 2H), 2.99-3.03 (m, 1H), 2.10- 2.47 (m, 6H), 1.87-2.01 (m, 3H), 1.44 (t, 3H). |
| 659a | 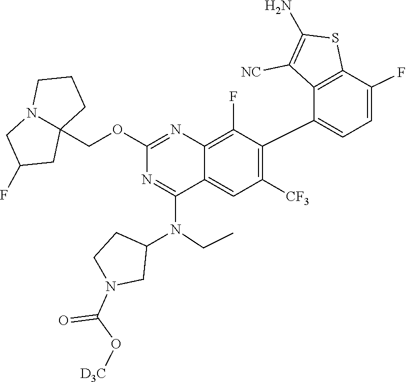 [SMILES: [2H]C([2H])([2H])OC(=O)N1CCC(N(CC)c2nc(OCC34CCCN3CC(F)C4)nc3c(F)c(-c4ccc(F)c5sc(N)c(C#N)c45)c(C(F)(F)F)cc23)C1] | |
| 660 | 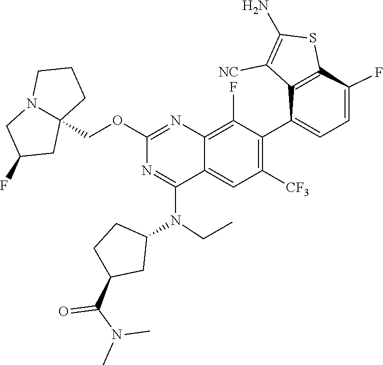 | LCMS m/z [M + 1]: 746.3 ¹H NMR (400 MHz, CD3OD): 8.23 (s, 1 H), 7.19- 7.23 (m, 1 H), 6.98-7.03 (m, 1 H), 5.40 (s, 0.5 H), 5.26 (s, 0.5 H), 4.98-5.03 (m, 1 H), 4.25-4.41 (m, 2 H), 3.74-3.94 (m, 2 H), 3.36-3.55 (m, 2 H), 3.22-3.27 (m, 1 H), 3.01-3.18 (m, 4 H), 2.94 (s, 3 H), 1.60-2.46 (m, 13 H), 1.43-1.46 (m, 3 H). |
| 661 | 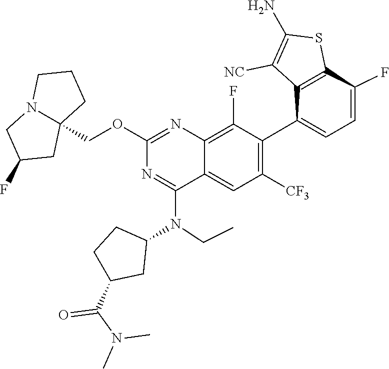 | LCMS m/z [M + 1]: 746.79 ¹H NMR (400 MHz, CD3OD) δ 8.22 (s, 1H), 7.22- 7.21 (m, 1H), 7.03-6.99 (m, 1H), 5.40 (s, 0.5H), 5.24 (s, 0.5H), 5.18.-4.87 (m, 1H), 4.42-4.34 (m, 1H), 4.34-4.22 (m, 1H), 3.87-3.80 (m, 2H), 3.47-3.30 (m, 3H), 3.12 (s, 3H), 3.02 (m, 1H), 2.97 (s, 3H), 2.78- 1.89 (m, 13H), 1.43-1.285 (m, 3H). |
| 661a | 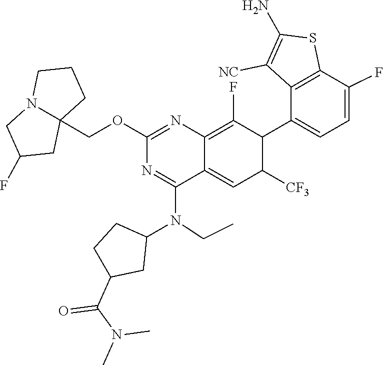 [SMILES: CCN(c1nc(OCC23CCCN2CC(F)C3)nc2c1=CC(C(F)(F)F)C(c1ccc(F)c3sc(N)c(C#N)c13)C=2F)C1CCC(C(=O)N(C)C)C1] | |
| 662 | 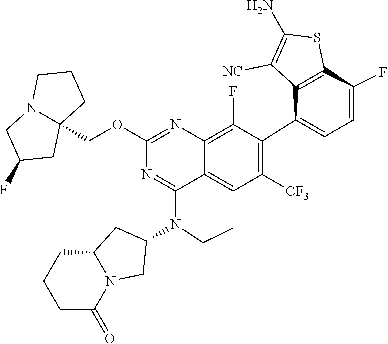 | |
| 663 | 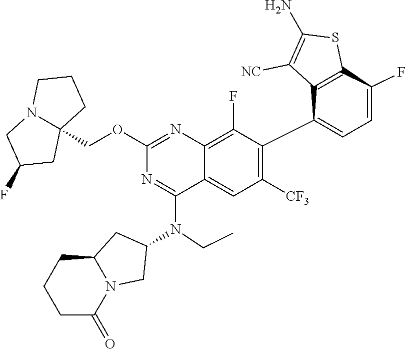 | |
| 663a | 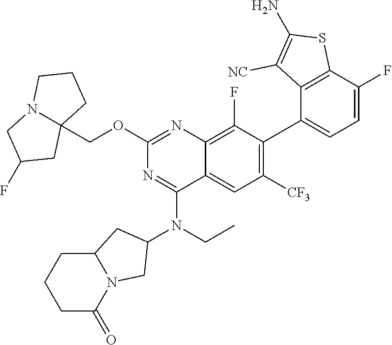 [SMILES: CCN(c1nc(OCC23CCCN2CC(F)C3)nc2c(F)c(-c3ccc(F)c4sc(N)c(C#N)c34)c(C(F)(F)F)cc12)C1CC2CCCC(=O)N2C1] | |
| 664 | 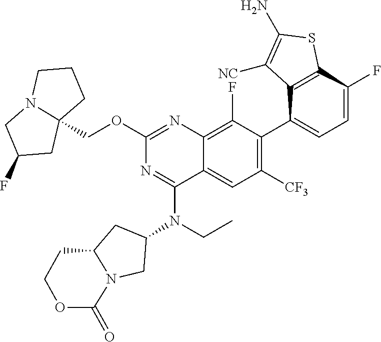 | LCMS m/z [M + 1]: 746.1 ¹H NMR (400 MHz, CD3OD) δ 8.14-8.25 (m, 1 H), 7.17-7.25 (m, 1 H), 6.96-7.05 (m, 1 H), 5.37 (s, 0.5 H), 5.24 (s, 0.5 H), 4.88-5.02 (m, 1 H), 4.11-4.33 (m, 3 H), 3.59-3.95 (m, 4 H), 3.37-3.52 (m, 1 H), 3.16- 3.29 (m, 4 H), 2.97-3.08 (m, 1 H), 2.90-2.96 (m, 3 H), 2.10-2.60 (m, 6 H), 1.94-2.05 (m, 2 H), 1.56-1.91 (m, 2 H), 1.40-1.50 (m, 3 H). |
| 665 | 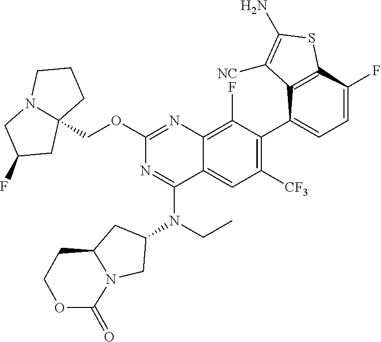 | LCMS m/z [M + 1]: 746.1 ¹H NMR (400 MHz, CD3OD): 8.22 (s, 1 H), 7.17- 7.27 (m, 1 H), 6.96-7.06 (m, 1 H), 5.38 (s, 0.5 H), 5.24 (s, 0.5 H), 4.91-5.02 (m, 1 H), 4.38-4.49 (m, 1 H), 4.25-4.35 (m, 2 H), 4.16-4.24 (m, 1 H), 3.78-3.97 (m, 4 H), 3.31-3.38 (m, 1 H), 3.16-3.29 (m, 3 H), 2.97-3.08 (m, 1H), 2.45-2.55 (m, 1H), 2.09-2.41 (m, 5H), 1.83-2..07 (m, 4H), 1.40-1.52 (m, 3H). |
| 665a | 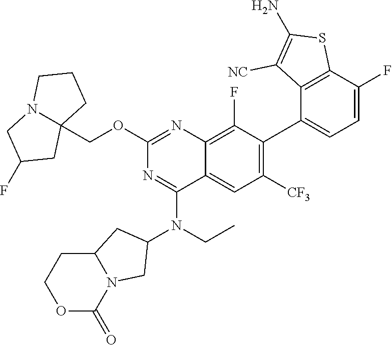 [SMILES: CCN(c1nc(OCC23CCCN2CC(F)C3)nc2c(F)c(-c3ccc(F)c4sc(N)c(C#N)c34)c(C(F)(F)F)cc12)C1CC2CCOC(=O)N2C1] | |
| 666 | 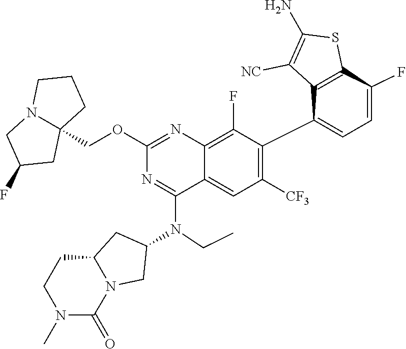 | LCMS m/z [M + 1]: 759.2 ¹H NMR (400 MHz, CD3OD) δ 8.14-8.25 (m, 1 H), 7.17-7.25 (m, 1 H), 6.96-7.05 (m, 1 H), 5.37 (s, 0.5 H), 5.24 (s, 0.5 H), 4.88-5.02 (m, 1 H), 4.11-4.33 (m, 3 H), 3.59-3.95 (m, 4 H), 3.37-3.52 (m, 1 H), 3.16- 3.29 (m, 4 H), 2.97-3.08 (m, 1 H), 2.90-2.96 (m, 3 H), 2.10-2.60 (m, 6 H), 1.94-2.05 (m, 2 H), 1.56-1.91 (m, 2 H), 1.40-1.50 (m, 3 H). |
| 667 | 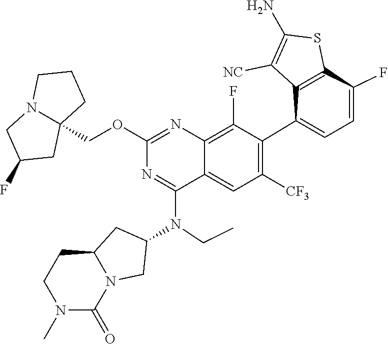 | LCMS m/z [M + 1]: 759.2 ¹H NMR (400 MHz, CD3OD): 8.16-8.25 (m, 1 H), 7.18-7.24 (m, 1 H), 6.97-7.04 (m, 1 H), 5.37 (s, 0.5 H), 5.23 (s, 0.5 H), 4.91-5.00 (m, 1 H), 4.08-4.32 (m, 3 H), 3.71-4.02 (m, 3 H), 3.55-3.67 (m, 1 H), 3.39- 3.49 (m, 1 H), 3.11-3.28 (m, 4 H), 2.98-3.08 (m, 1 H), 2.93 (s, 1 H), 2.13-2.66 (m, 6 H), 1.89-2.05 (m, 3 H), 1.58-1.72 (m, 1 H), 1.38-1.48 (m, 3 H). |
| 667a | 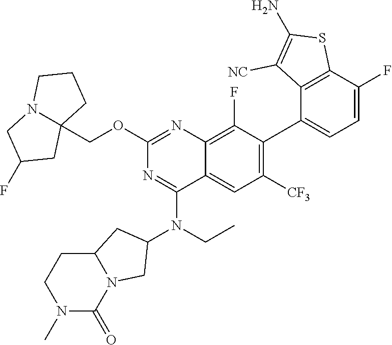 [SMILES: CCN(c1nc(OCC23CCCN2CC(F)C3)nc2c(F)c(-c3ccc(F)c4sc(N)c(C#N)c34)c(C(F)(F)F)cc12)C1CC2CCN(C)C(=O)N2C1] | |
| 682 | 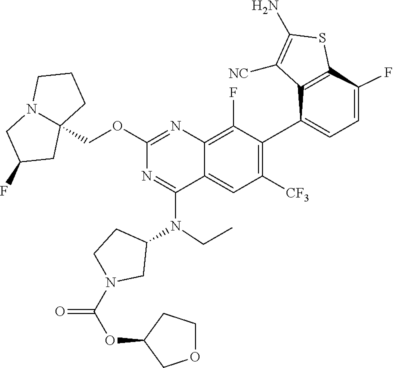 | LCMS m/z [M + 1]: 790.20 1H NMR (400 MHz, CD3OD) δ 8.26 (s, 1H), 7.21 (t, 1H), 7.01 (t, 1H), 5.50 (s, 0.5H), 5.37 (s, 0.5H), 5.24 (s, 1H), 5.04 (s, 1H), 4.51 (d, 1H), 4.42 (t, 1H), 3.95- 3.84 (m, 7H), 3.75 (t, 1H), 3.65-3.37 (m, 5H), 3.25 (s, 1H), 2.50-2.36 (m, 4H), 2.30-2.26 (m, 1H), 2.17 (s, 1H), 2.08-1.97 (s, 2H), 1.46 (t, 3H). |
| 683 | 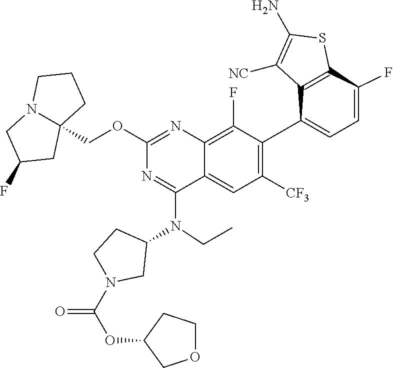 | LCMS m/z [M + 1]: 790.2 1H NMR (400 MHz, CD3OD) δ 8.23 (s, 1H), 7.25- 7.18 (m, 1H), 7.01 (t, 1H), 5.39 (m, 0.5H), 5.28-5.22 (m, 1.5H), 5.06-4.98 (m, 1H), 4.36-4.24 (m, 2H), 3.97-3.69 (m, 8H), 3.62-3.35 (m, 3H), 3.29-3.22 (m, 2H), 3.09-3.02 (m, 1H), 2.50-2.29 (m, 3H), 2.27-2.12 (m, 3H), 2.08-1.87 (m, 4H), 1.44 (t, 3H). |
| 683a | 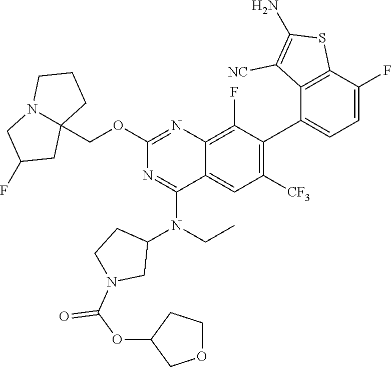 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2CCOC2)C1] | |
| 684 | 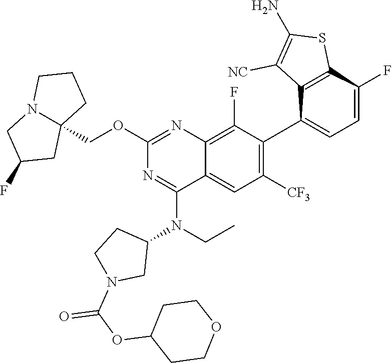 | LCMS m/z [M + 1]: 804.3 1H NMR (400 MHz, CD3OD) δ 8.23 (s, 1H), 7.21 (t, 1H), 7.00 (t, 1H), 5.37 (s, 0.5H), 5.23 (s, 0.5H), 5.00- 5.04 (m, 1H), 4.83-4.84 (m, 1H), 4.22-4.32 (m, 2H), 3.84-3.88 (m, 5H), 3.74-3.76 (m, 1H), 3.56-3.58 (m, 3H), 3.42-3.47 (m, 1H), 3.20-3.24 (m, 3H), 2.98-3.02 (m, 1H), 2.45-2.47 (m, 1H), 2.10-2.36 (m, 4H), 1.86- 2.00 (m, 5H), 1.68 (s, 2H), 1.44 (t, 3H). |
| 684a | 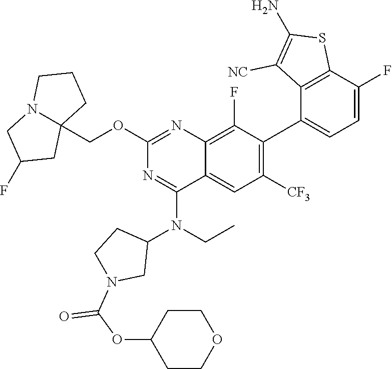 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2CCOCC2)C1] | |
| 686 | 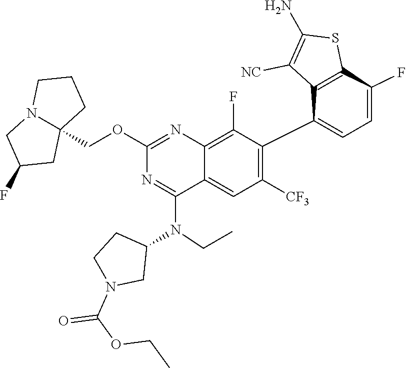 | LCMS m/z [M + 1]: 748.2 1H NMR (400 MHz, CD3OD) δ = 8.23 (s, 1H), 7.25- 7.17 (m, 1H), 7.01 (t, J = 8.9, 1H), 5.38 (s, 0.5H), 5.25 (s, 0.5H), 5.08-4.95 (m, 1H), 4.29 (q, J = 10.7, 2H), 4.15 (q, J = 6.9, 2H), 3.98-3.69 (m, 4H), 3.63-3.52 (m, 1H), 3.44 (s, 2H), 3.25 (s, 2H), 3.09-2.99 (m, 1H), 2.51-2.10 (m, 5H), 2.05-1.85 (m, 3H), 1.45 (t, J = 6.8, 3H), 1.28 (s, 3H). |
| 686a | 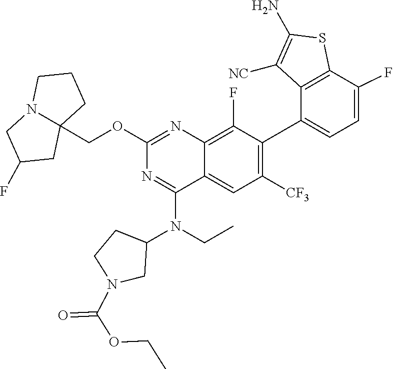 [SMILES: CCOC(=O)N1CCC(N(CC)c2nc(OCC34CCCN3CC(F)C4)nc3c(F)c(-c4ccc(F)c5sc(N)c(C#N)c45)c(C(F)(F)F)cc23)C1] | |
| 687 | 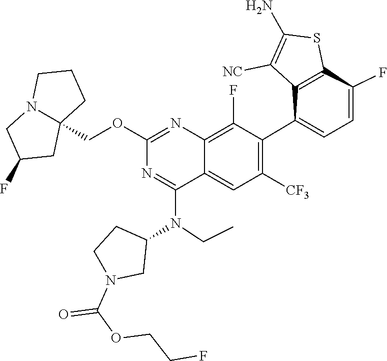 | LCMS m/z [M + 1]: 766.1. 1H NMR (400 MHz, CD3OD) δ8.23 (s, 1H), 7.23- 7.20 (m, 1H), 7.01 (t, J = 8.8 Hz, 1H), 5.37-5.24 (d, 1H), 5.04-5.00 (m, 1H), 4.66 (s, 1H), 4.54 (s, 1H), 4.37-4.23(m, 4H), 3.99-3.77 (m, 4H), 3.61-3.44 (m, 2H), 3.25-3.21 (m, 2H), 3.02-3.01(m, 1H), 2.49- 1.87(m, 8H), 1.45(t, J = 7.2 Hz, 3H). |
| 687a | 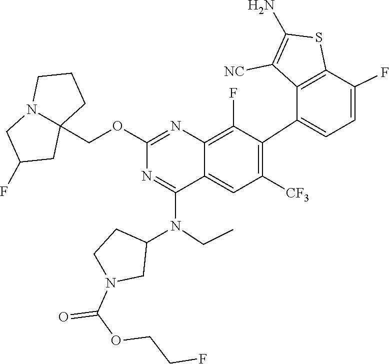 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OCCF)C1] | |
| 688 | 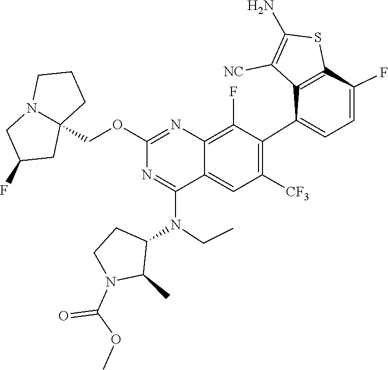 | LCMS m/z [M + 1]: 748.1 1H NMR (400 MHz, CD3OD) δ 8.21 (s, 1H), 7.20- 7.24 (m, 1H), 7.01 (t, 1H), 5.37 (s, 0.5H), 5.24 (s, 0.5H), 4.58-4.63 (m, 1H), 4.20-4.27 (m, 3H), 3.76- 3.92 (m, 3H), 3.72 (s, 3H), 3.36-3.49 (m, 1H), 3.12- 3.26 (m, 3H), 2.99-3.05 (m, 1H), 2.46-2.55 (m, 1H), 2.09-2.38 (m, 4H), 1.85-2.01 (m, 3H), 1.43 (t, 3H), 1.36 (d, 3H). |
| 688a | 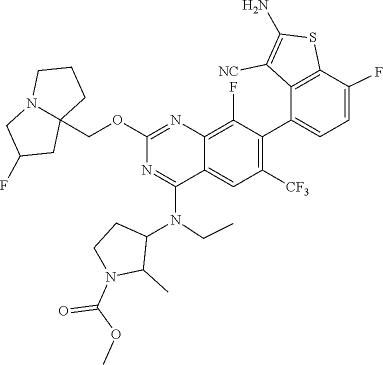 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC)C1C] | |
| 689 | 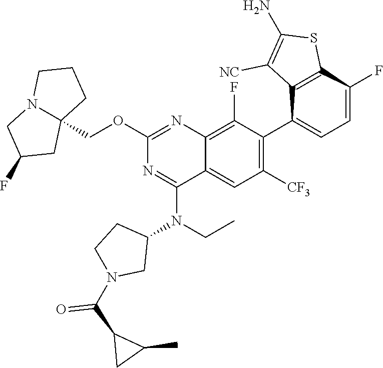 | LCMS m/z [M + 1]: 758.3 1H NMR (400 MHz, CD3OD) δ 8.25 (s, 1H), 7.25- 7.17 (m, 1H), 7.05-6.97 (m, 1H), 5.37 (s, 0.5H), 5.24 (s, 0.5H), 5.10-5.01 (m, 1H), 4.34-4.27 (m, 1H), 4.25-4.17 (m, 1H), 4.05-3.67 (m, 5H), 3.65-3.54 (m, 1H), 3.49-3.33 (m, 2H), 3.28-3.20 (m, 2H), 3.07-2.99 (m, 1H), 2.55-1.80 (m, 10H), 1.50-1.36 (m, 3H), 1.33-1.26 (m, 1H), 1.11-1.06 (m, 2H), 0.96-0.87 (m, 2H). |
| 690 | 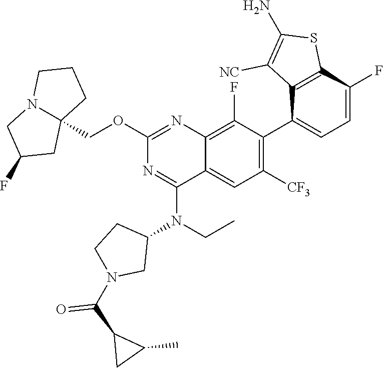 | LCMS m/z [M + 1]: 758.2 1H NMR (400 MHz, CD3OD) δ 8.25 (d, 1H), 7.20- 7.23 (m, 1H), 7.01 (t, 1H), 5.38 (s, 0.5H), 5.25 (s, 0.5H), 5.00-5.11 (m, 1H), 4.22-4.34 (m, 2H), 3.74- 4.06 (m, 5H), 3.38-3.62 (m, 2H), 3.22-3.24 (m, 3H), 3.01-3.07 (m, 1H), 2.13-2.61 (m, 5H), 1.87-2.03 (m, 3H), 1.53-1.60 (m, 1H), 1.43-1.49 (m, 3H), 1.08-1.17 (m, 4H), 0.64-0.70 (m, 1H). |
| 691 | 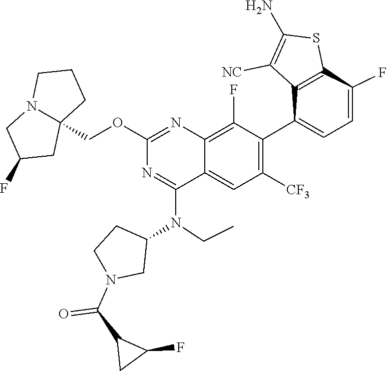 | LCMS m/z [M + 1]: 762.1 1H NMR (400 MHz, CD3OD) δ 8.30-8.21 (m, 1H), 7.26-7.18 (m, 1H), 7.01 (t, 1H), 5.42 (s, 0.5H), 5.28 (s, 0.5H), 5.14-5.06 (m, 1H), 5.02-4.92 (m, 1H), 4.44-4.20 (m, 3H), 4.08-3.62 (m, 6H), 3.47-3.35 (m, 2H), 3.12-3.05 (m, 1H), 2.60-2.42 (m, 2H), 2.39-2.17 (m, 3H), 2.12-1.90 (m, 4H), 1.76-1.68 (m, 1H), 1.56- 1.38 (m, 3H), 1.13-1.06 (m, 1H). |
| 692 | 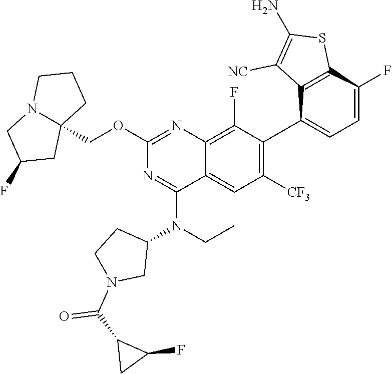 | LCMS m/z [M + 1]: 762.2 1H NMR (400 MHz, CD3OD) δ 8.25 (d, 1H), 7.25- 7.18 (m, 1H), 7.01 (t, 1H), 5.42-5.38 (m, 0.5H), 5.29- 5.23 (m, 0.5H), 5.13-5.01 (m, 1H), 4.92-4.87 (m, 0.5H), 4.76-4.69 (m, 0.5H), 4.35-4.26 (m, 2H), 4.14- 4.07 (m, 0.5H), 4.04-3.75 (m, 4H), 3.61-3.55 (m, 0.5H), 3.49-3.32 (m, 2H), 3.29-3.21 (m, 2H), 3.10- 3.01 (m, 1H), 2.54-1.84 (m, 9H), 1.51-1.43 (m, 3H), 1.33-1.23 (m, 2H). |
| 692a | 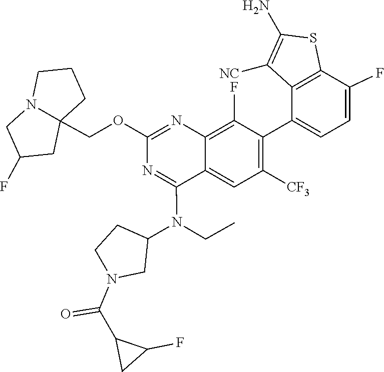 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CC2F)C1] | |
| 693 | 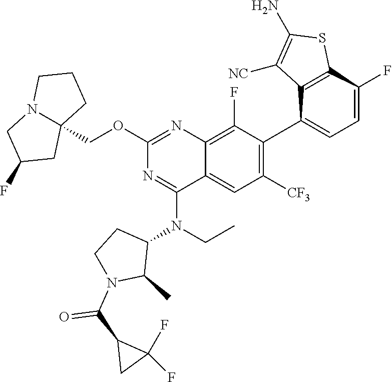 | LCMS m/z [M + 1]: 794.1 1H NMR (400 MHz, CD3OD) δ 8.22 (s, 1H), 7.20- 7.23 (m, 1H), 7.01 (t, 1H), 5.36 (s, 0.5H), 5.22 (s, 0.5H), 4.74-4.79 (m, 0.5H), 4.63-4.67 (m, 1H), 4.44- 4.50 (m, 0.5H), 4.21-4.32 (m, 2H), 4.05-4.09 (m, 0.5H), 3.90-3.95 (m, 1H), 3.76-3.85 (m, 2H), 3.46- 3.53 (m, 0.5H), 3.12-3.25 (m, 3H), 2.80-3.01 (m, 2H), 2.31-2.63 (m, 3H), 2.08-2.25 (m, 3H), 1.95-2.00 (m, 2H), 1.69-1.88 (m, 2H), 1.45-1.49 (m, 3H), 1.35- 1.38 (m, 3H). |
| 693a | 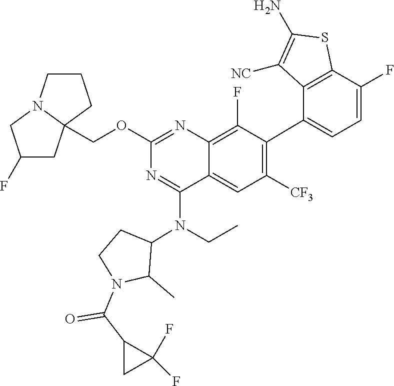 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CC2(F)F)C1C] | |
| 694 | 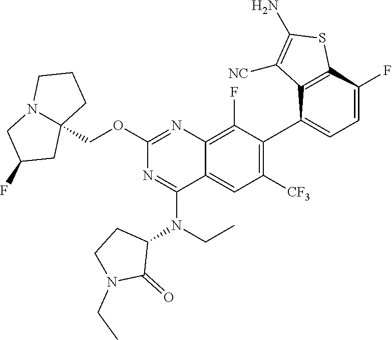 | LCMS m/z [M + 1]: 718.2 1H NMR (400 MHz, CD3OD) δ = 8.28 (s, 1H), 7.23 (dd, J = 8.1, 5.1, 1H), 7.02 (t, J = 8.9, 1H), 5.43 (s, 0.5H), 5.29 (s, 0.5H), 4.70 (s, 1H), 4.28 (q, J = 11.2, 2H), 4.06-3.79 (m, 2H), 3.70-3.37 (m, 6H), 3.24- 3.14 (m, 2H), 2.58-1.93 (m, 8H), 1.57 (t, J = 7.0, 3H), 1.22 (t, J = 7.2, 3H). |
| 695 | 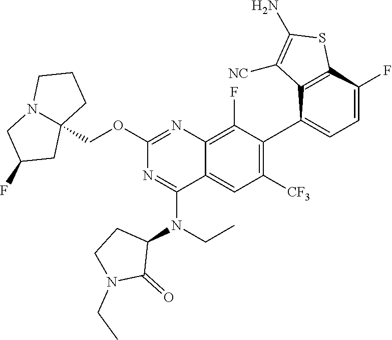 | LCMS m/z [M + 1]: 718.2 1H NMR (400 MHz, CD3OD) δ 8.49 (brs, 0.38H), 8.31 (s, 1H), 7.23-7.16 (m, 1H), 7.00 (t, J = 8.8 Hz, 1H), 5.47 (s, 0.54H), 5.34 (s, 0.56H), 4.82-4.75 (m, 1H), 4.49-4.42 (m, 1H), 4.32-4.25 (m, 1H), 4.08-3.95 (m, 1H), 3.91-3.81 (m, 1H), 3.66-3.49 (m, 6H), 3.27- 3.12 (m, 2H), 2.60-2.35 (m, 4H), 2.30-1.96 (m, 4H), 1.54 (t, J = 7.0 Hz, 3H), 1.23 (t, J = 7.4 Hz, 3H). |
| 695a | 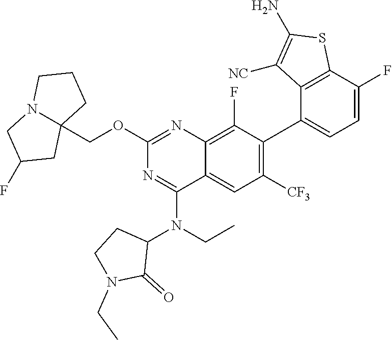 [SMILES: CCN1CCC(N(CC)c2nc(OCC34CCCN3CC(F)C4)nc3c(F)c(-c4ccc(F)c5sc(N)c(C#N)c45)c(C(F)(F)F)cc23)C1=O] | |
| 696 | 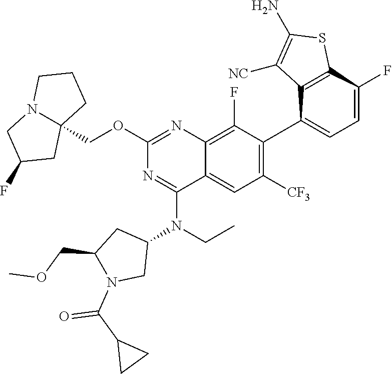 | |
| 696a | 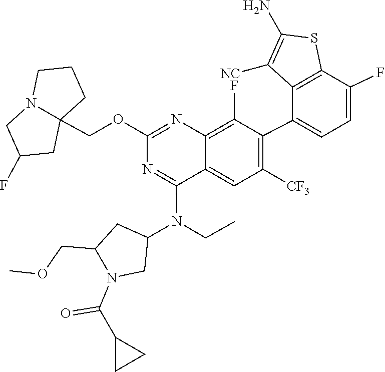 [SMILES: CCN(c1nc(OCC23CCCN2CC(F)C3)nc2c(F)c(-c3ccc(F)c4sc(N)c(C#N)c34)c(C(F)(F)F)cc12)C1CC(COC)N(C(=O)C2CC2)C1] | |
| 697 | 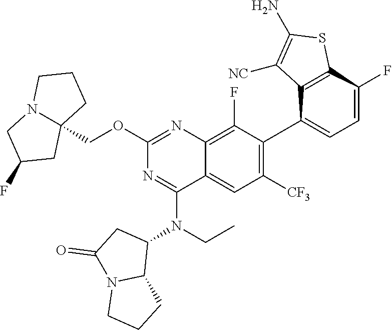 | |
| 697a | 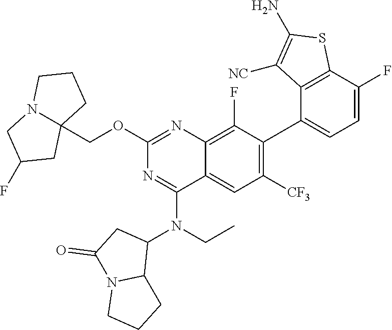 [SMILES: CCN(c1nc(OCC23CCCN2CC(F)C3)nc2c(F)c(-c3ccc(F)c4sc(N)c(C#N)c34)c(C(F)(F)F)cc12)C1CC(=O)N2CCCC12] | |
| 698 | 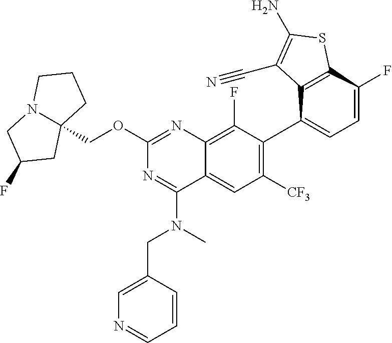 | |
| 698a | 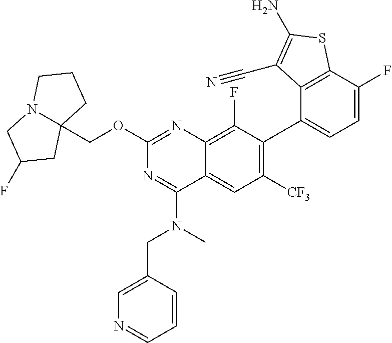 [SMILES: CN(Cc1cccnc1)c1nc(OCC23CCCN2CC(F)C3)nc2c(F)c(-c3ccc(F)c4sc(N)c(C#N)c34)c(C(F)(F)F)cc12] | |

Table 9 includes selected compounds of the present disclosure.

TABLE 9
Selected compounds of the present disclosure.

| Cmpd. | | |
|---|---|---|
| No. | Structure | Analytical data |
| 668 | 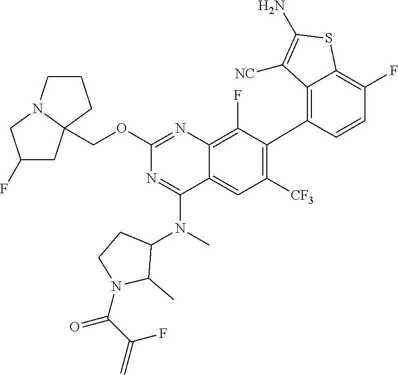 | LCMS m/z [M + 1]: 742.1 1H NMR (400 MHz, CD3OD) δ 8.34 (s, 1H), 7.20- 7.23 (m, 1H), 7.01 (t, 1H), 5.39 (s, 0.5H), 5.25 (s, 0.5H), 4.94-5.06 (m, 2H), 4.78-4.82 (m, 1H), 3.95- 4.36 (m, 2H), 3.66-3.81 (m, 1H), 3.60-3.63 (m, 3H), 3.37-3.54 (m, 1H), 3.31-3.24 (m, 2H), 3.00-3.06 (m, 1H), 2.60-2.65 (m, 1H), 2.36-2.48 (m, 2H), 2.20-2.32 (m, 1H), 2.11 (s, 1H), 1.98-2.06 (m, 5H), 1.90-1.93 (m, 1H), 1.20 (t, 3H). |
| 668a | 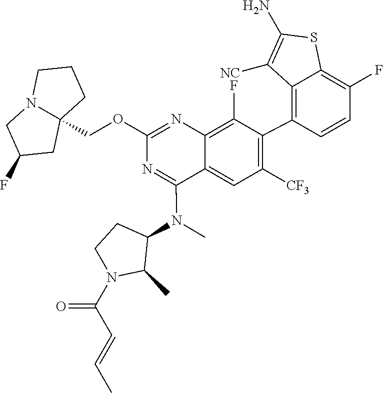 [SMILES: C/C=C/C(=O)N1CC[C@@H](N(C)c2nc(OC[C@@]34CCCN3C[C@H](F)C4)nc3c(F)c(-c4ccc(F)c5sc(N)c(C#N)c45)c(C(F)(F)F)cc23)[C@H]1C] | |
| 669 | 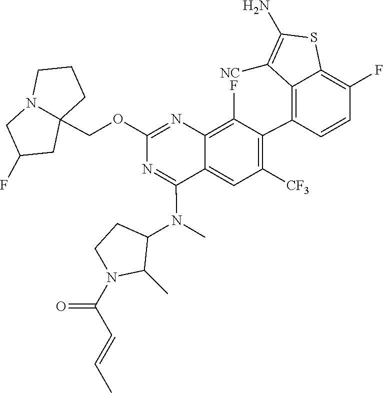 | LCMS m/z [M + 1]: 748.1 1H NMR (400 MHz, CD3OD) δ 8.36 (s, 1H), 7.28- 7.18 (m, 1H), 7.09-6.96 (m, 1H), 8.57-5.08 (m, 4H), 4.48 (s, 1H), 4.44-4.40 (m, 2H), 3.98-3.79 (m, 2H), 3.62 (s, 3H), 3.54-3.47 (m, 3H), 3.30-3.21 (m, 1H), 2.44-2.00 (m, 8H), 1.13 (s, 3H). |
| 669a | 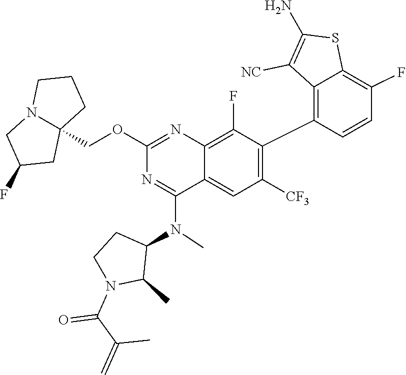 [SMILES: C=C(C)C(=O)N1CC[C@@H](N(C)c2nc(OC[C@@]34CCCN3C[C@H](F)C4)nc3c(F)c(-c4ccc(F)c5sc(N)c(C#N)c45)c(C(F)(F)F)cc23)[C@H]1C] | |
| 670 | 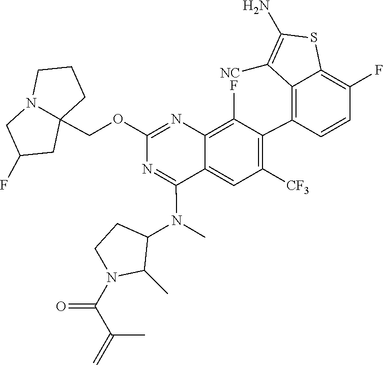 [SMILES: C=C(C)C(=O)N1CCC(N(C)c2nc(OCC34CCCN3CC(F)C4)nc3c(F)c(-c4ccc(F)c5sc(N)c(C#N)c45)c(C(F)(F)F)cc23)C1C] | LCMS m/z [M + 1]: 744.1 1H NMR (400 MHz, CD3OD) δ 8.41-8.37 (m, 1H), 7.22 (s, 1H), 7.06-6.92 (m, 2H), 6.35-6.31 (m, 1H), 5.48-5.35 (m, 1H), 5.02 (s, 1H), 4.93-4.90 (m, 3H), 4.48-4.38 (m, 2H), 3.93-3.53 (m, 7H), 2.70-1.96 (m, 11H), 1.14-1.10 (m, 3H). |
| 670a | 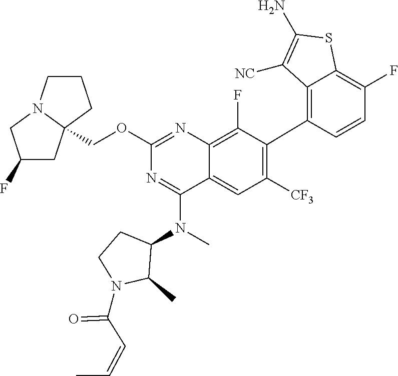 [SMILES: C/C=C\C(=O)N1CC[C@@H](N(C)c2nc(OC[C@@]34CCCN3C[C@H](F)C4)nc3c(F)c(-c4ccc(F)c5sc(N)c(C#N)c45)c(C(F)(F)F)cc23)[C@H]1C] | |
| 671 | 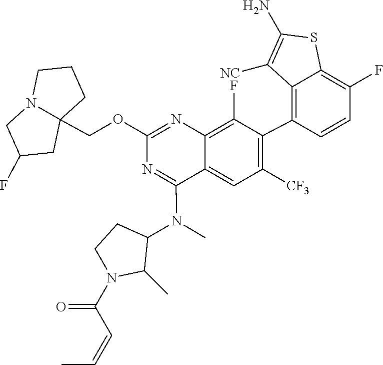 [SMILES: C/C=C\C(=O)N1CCC(N(C)c2nc(OCC34CCCN3CC(F)C4)nc3c(F)c(-c4ccc(F)c5sc(N)c(C#N)c45)c(C(F)(F)F)cc23)C1C] | LCMS m/z [M + 1]: 744.1 1H NMR (400 MHz, CD3OD) δ 8.34 (s, 1H), 7.21- 7.20 (m, 1H), 7.01 (t, 1H), 5.88 (s, 0.5H), 5.34 (d, 1H), 5.30 (s, 1.5H), 5.19 (s, 1H), 5.02 (s, 1H), 4.42- 4.33 (m, 2H), 3.83 (s, 1H), 3.63-3.60 (m, 5H), 3.47 (m, 1H), 3.30 (s, 1H), 2.16 (s, 1H), 2.14-1.89 (m, 11H), 1.10 (s, 3H). |
| 671a | 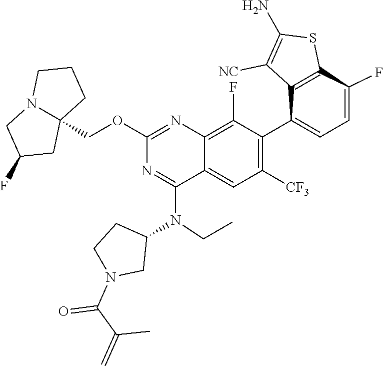 | |
| 672 | 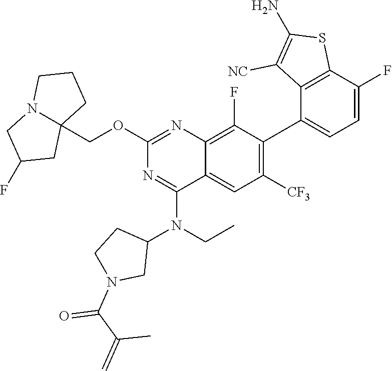 [SMILES: C=C(C)C(=O)N1CCC(N(CC)c2nc(OCC34CCCN3CC(F)C4)nc3c(F)c(-c4ccc(F)c5sc(N)c(C#N)c45)c(C(F)(F)F)cc23)C1] | LCMS m/z [M + 1]: 744.1 1H NMR (400 MHz, CD3OD) δ 8.34 (s, 1H), 7.21 (s, IH), 7.01 (t, 1H), 6.08-6.33 (m, 2H), 5.37 (d, 0.5H), 5.25 (d, 0.5H), 4.96-5.04 (m, 1H), 4.27-4.43 (m, 2H), 3.42-3.94 (m, 7H), 3.04-3.12 (m, 3H), 2.18-2.70 (m, 5H), 1.91-2.02 (m, 6H), 1.07-1.14 (m, 3H). |
| 672a | 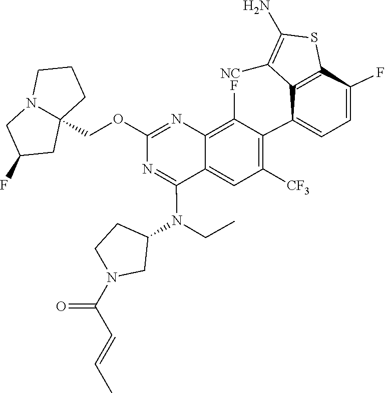 | |
| 673 | 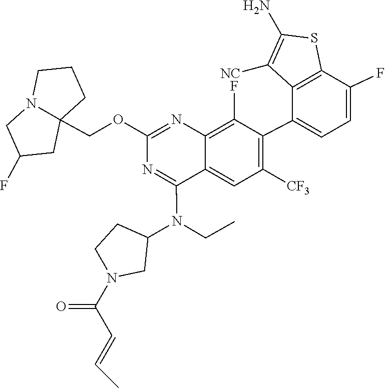 [SMILES: C/C=C/C(=O)N1CCC(N(CC)c2nc(OCC34CCCN3CC(F)C4)nc3c(F)c(-c4ccc(F)c5sc(N)c(C#N)c45)c(C(F)(F)F)cc23)C1] | LCMS m/z [M + 1]: 744.4 1H NMR (400 MHz, CD3OD) δ 8.43 (s, 1H), 8.30- 8.23 (m, 1H), 7.25-7.18 (m, 1H), 7.02 (t, 1H), 5.51 (s, 0.5H), 5.40-5.33 (m, 1.5H), 5.25 (s, 1H), 5.14- 4.97 (m, 2H), 4.60-4.54 (m, 1H), 4.50-4.40 (m, 1H), 4.10-4.01 (m, 1H), 3.94-3.55 (m, 8H), 2.59-2.11 (m, 8H), 2.08-1.91 (m, 4H), 1.54-1.40 (m, 3H). |
| 673a | 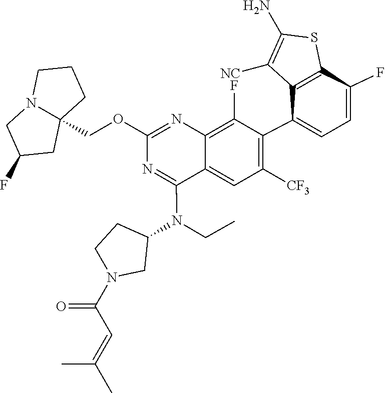 | |
| 674 | 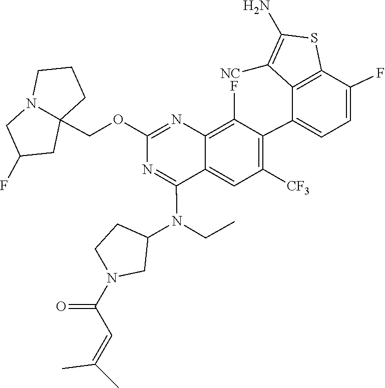 | LCMS m/z [M + 1]: 744.10 1H NMR (400 MHz, CD3OD) δ 8.27 (s, 1H), 7.22 (t, 1H), 7.02 (t, 1H), 6.93-6.88 (m, 0.5H), 6.38-6.29 (m, 1H), 5.52 (s, 0.5H), 5.39 (s, 0.5H), 5.05-4.99 (m, 1H), 4.55-4.42(m, 2H), 4.12-3.86 (m, 5H), 3.76-3.39 (m, 5H), 2.58-2.36 (m, 4H), 2.30-2.28 (m, 1H), 2.23- 2.13 (m, 2H), 2.03 (s, 1H), 1.94-1.89 (m, 3H), 1.52- 1.46 (m, 3H). |
| 674a | 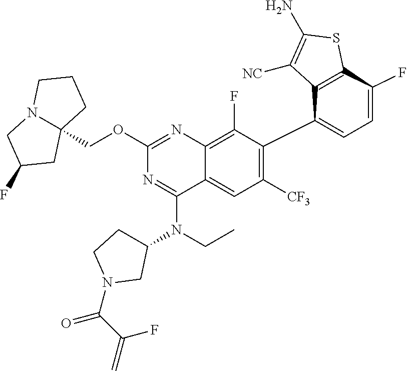 | |
| 675 | 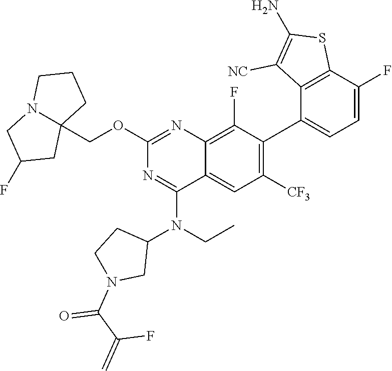 | LCMS m/z [M + 1]: 758.2 1H NMR (400 MHz, CD3OD) δ 8.23 (s, 1H), 7.19- 7.23 (m, 1H), 7.01 (t, 1H), 5.95 (d, 1H), 5.37 (s, 0.5H), 5.23 (s, 0.5H), 4.98-5.06 (m, 1H), 4.22-4.31 (m, 2H), 3.99-4.06 (m, 1H), 3.84-3.88 (m, 3H), 3.44- 3.73 (m, 2H), 3.21-3.26 (m, 3H), 2.99-3.06 (m, 1H), 2.21-2.53 (m, 4H), 1.95-2.13 (m, 6H), 1.90 (d, 4H), 1.45 (t, 3H). |
| 675a | 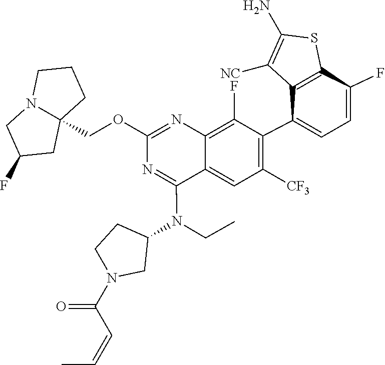 | |
| 676 | 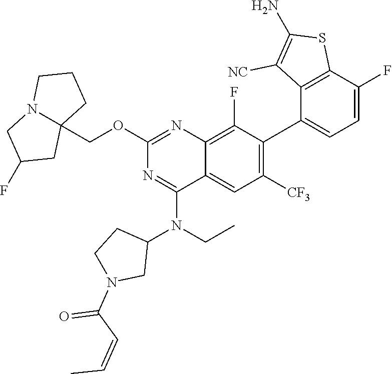 | LCMS m/z [M + 1]: 748.1 1H NMR (400 MHz, CD3OD) δ 8.24 (s, 1H), 7.20- 7.23 (m, 1H), 7.01 (t, 1H), 5.54 (s, 0.5H), 5.42 (s, 0.5H), 5.23-5.36 (m, 2H), 4.98-5.05 (m, 1H), 4.20- 4.31 (m, 2H), 3.72-4.09 (m, 5H), 3.47-3.59 (m, 1H), 3.12-3.25 (m, 3H), 2.98-3.05 (m, 1H), 2.20-2.52 (m, 4H), 2.09-2.16 (m, 1H), 1.86-2.02 (m, 3H), 1.46 (t, 3H). |
| 676a | 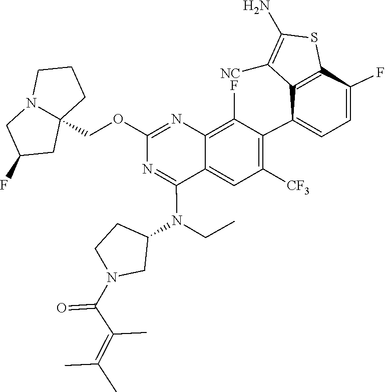 | |
| 677 | 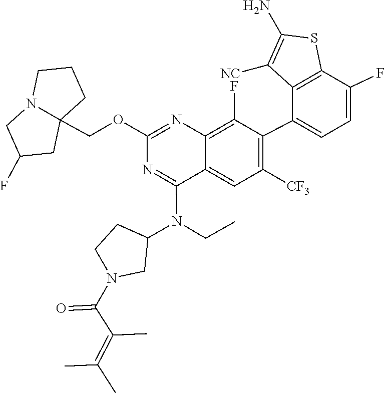 | LCMS m/z [M + 1]: 744.2 1H NMR (400 MHz, CD3OD) δ 8.47 (s, 1H), 8.27 (s, 1H), 7.26-7.17 (m, 1H), 7.01 (t, 1H), 6.96-6.83 (m, 1H), 6.41-6.27 (m, 1H), 5.50 (s, 0.5H), 5.37 (s, 0.5H), 5.08-4.98 (m, 1H), 4.57-4.28 (m, 3H), 4.14- 3.83 (m, 4H), 3.75-3.66 (m, 2H), 3.61-3.44 (m, 3H), 2.64-2.32 (m, 4H), 2.31-2.12 (m, 3H), 2.06-1.87 (m, 4H), 1.51-1.43 (m, 3H). |
| 677a | 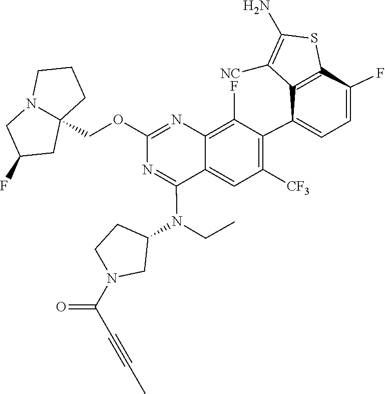 | |
| 678 | 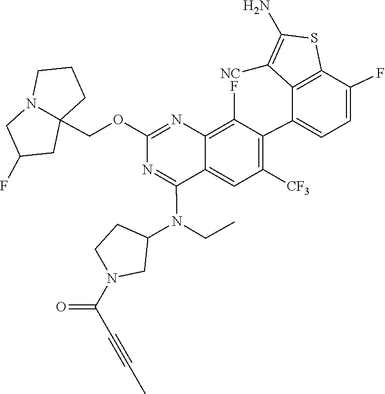 [SMILES: CC#CC(=O)N1CCC(N(CC)c2nc(OCC34CCCN3CC(F)C4)nc3c(F)c(-c4ccc(F)c5sc(N)c(C#N)c45)c(C(F)(F)F)cc23)C1] | |
| 678a | 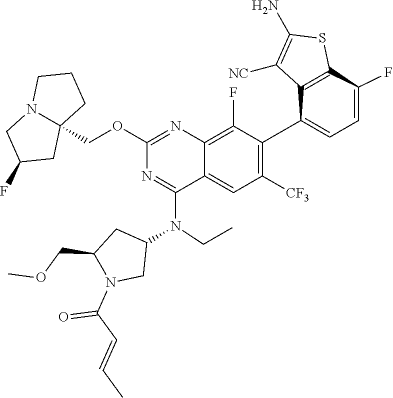 | |
| 679 | 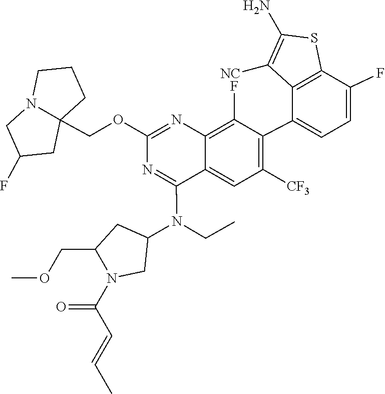 | LCMS m/z [M + 1]: 742.3 ¹H NMR (400 MHz, CD3OD) δ = 8.23 (d, J = 5.8, 1H), 7.27-7.17 (m, 1H), 7.01 (t, J = 8.9, 1H), 5.39 (s, 0.5H), 5.25 (s, 0.5H), 4.97 (dd, J= 17.3, 8.8, 1H), 4.34-4.19 (m, 2H), 4.00 (dd, J = 37.3, 33.1, 1H), 3.92-3.56 (m, 4H), 3.41 (d, J = 56.4, 2H), 3.27-2.94 (m, 3H), 2.57-2.12 (m, 5H), 2.09-1.84 (m, 6H), 1.48 (td, J = 6.8, 3.4, 3H). |
| 679a | 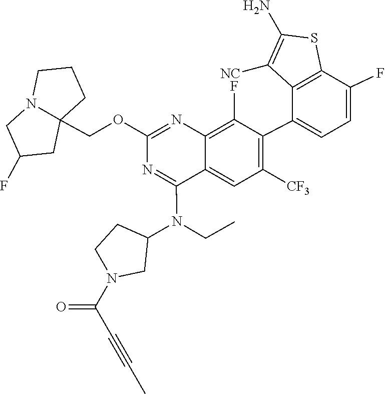 [SMILES: CC#CC(=O)N1CCC(N(CC)c2nc(OCC34CCCN3CC(F)C4)nc3c(F)c(-c4ccc(F)c5sc(N)c(C#N)c45)c(C(F)(F)F)cc23)C1] | |
| 845 | 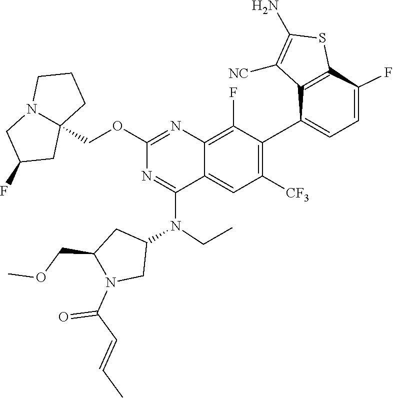 | LCMS m/z [M + 1]: 788.2 ¹H NMR (400 MHz, CD3OD) δ 8.21 (d, 1H), 7.24- 7.17 (m, 1H), 7.01 (t, 1H), 6.96-6.85 (m, 1H), 6.45- 6.29 (m, 1H), 5.38-5.22 (m, 2H), 4.55-4.46 (m, 1H), 4.29-4.19 (m, 2H), 4.07-3.92 (m, 1H), 3.91-3.85 (m, 2H), 3.77-3.69 (m, 1H), 3.51-3.44 (m, 1H), 3.33 (d, 3H), 3.27-3.11 (m, 3H), 3.05-2.98 (m, 1H), 2.80-2.65 (m, 1H), 2.41-1.80 (m, 11H), 1.47-1.39 (m, 3H). |
| 845a | 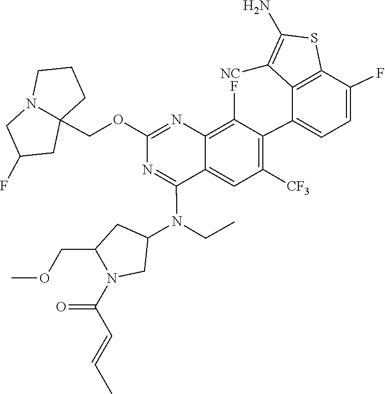 [SMILES: C/C=C/C(=O)N1CC(N(CC)c2nc(OCC34CCCN3CC(F)C4)nc3c(F)c(-c4ccc(F)c5sc(N)c(C#N)c45)c(C(F)(F)F)cc23)CC1COC] | |

TABLE 10
Selected compounds of the present disclosure.

| Cmpd. No. | Structure | Analytical Data |
|---|---|---|
| 682 | 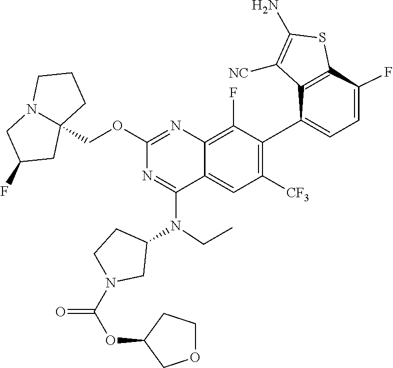 | LCMS m/z [M − 1]: 790.20 ¹H NMR (400 MHz, CD₃OD) δ 8.26 (s, 1H), 7.21 (t, 1H), 7.01 (t, 1H), 5.50 (s, 0.5H), 5.37 (s, 0.5H), 5.24 (s, 1H), 5.04 (s, 1H), 4.51 (d, 1H), 4.42 (t, 1H), 3.95-3.84 (m, 7H), 3.75 (t, 1H), 3.65-3.37 (m, 5H), 3.25 (s, 1H), 2.50-2.36 (m, 4H), 2.30-2.26 (m, 1H), 2.17 (s, 1H), 2.08- 1.97 (s, 2H), 1.46 (t, 3H). |
| 682a | 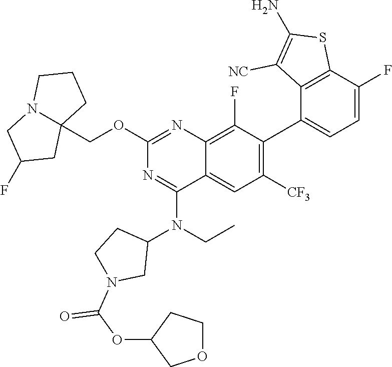 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2CCOC2)C1] | |
| 683 | 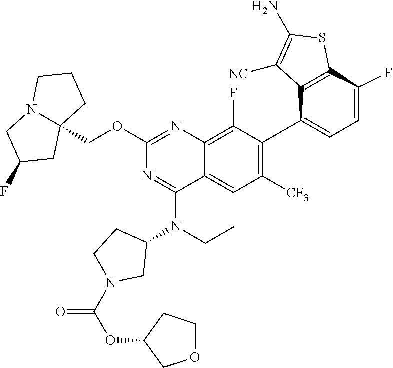 | LCMS m/z [M − 1]: 790.2 ¹H NMR (400 MHz, CD₃OD) δ 8.23 (s, 1H), 7.25-7.18 (m, 1H), 7.01 (t, 1H), 5.39 (m, 0.5H), 5.28-5.22 (m, 1.5H), 5.06-4.98 (m, 1H), 4.36-4.24 (m, 2H), 3.97-3.69 (m, 8H), 3.62-3.35 (m, 3H), 3.29-3.22 (m, 2H), 3.09- 3.02 (m, 1H), 2.50-2.29 (m, 3H), 2.27-2.12 (m, 3H), 2.08-1.87 (m, 4H), 1.44 (t, 3H). |
| 684 | 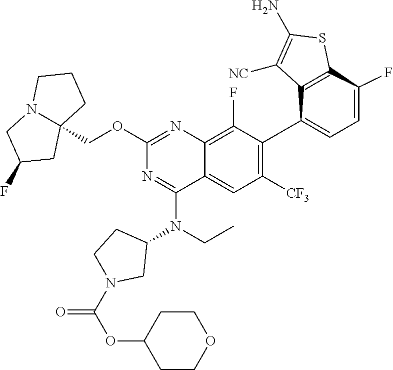 | LCMS m/z [M − 1]: 804.3 ¹H NMR (400 MHz, CD₃OD) δ 8.23 (s, 1H), 7.21 (t, 1H), 7.00 (t, 1H), 5.37 (s, 0.5H), 5.23 (s, 0.5H), 5.00- 5.04 (m, 1H), 4.83-4.84 (m, 1H), 4.22-4.32 (m, 2H), 3.84-3.88 (m, 5H), 3.74-3.76 (m, 1H), 3.56-3.58 (m, 3H), 3.42-3.47 (m, 1H), 3.20-3.24 (m, 3H), 2.98-3.02 (m, 1H), 2.45-2.47 (m, 1H), 2.10-2.36 (m, 4H), 1.86- 2.00 (m, 5H), 1.68 (s, 2H), 1.44 (t, 3H). |
| 684a | 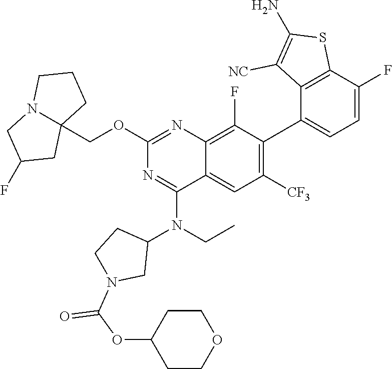 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2CCOCC2)C1] | |
| 685 | 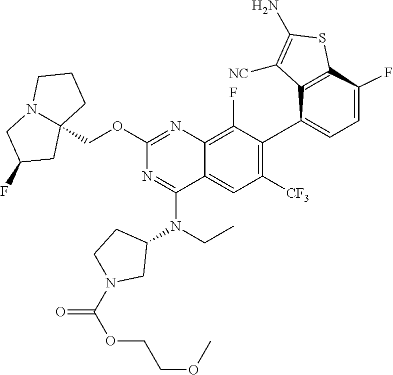 | LCMS m/z [M − 1]: 778.2 ¹H NMR (400 MHz, CD₃OD) δ 8.22 (s, 1H), 7.19-7.23 (m, 1H), 7.00 (t, 1H), 5.37 (s, 0.5H), 5.24 (s, 0.5H), 4.97-5.05 (m, 1H), 4.22-4.31 (m, 4H), 3.75-3.93 (m, 4H), 3.57-3.63 (m, 3H), 3.42-3.47 (m, 1H), 3.37 (d, 3H), 3.12-3.26 (m, 3H), 2.99-3.05 (m, 1H), 2.21-2.50 (m, 4H), 2.11-2.17 (m, 1H), 1.87-2.01 (m, 3H), 1.44 (t, 3H). |
| 685a | 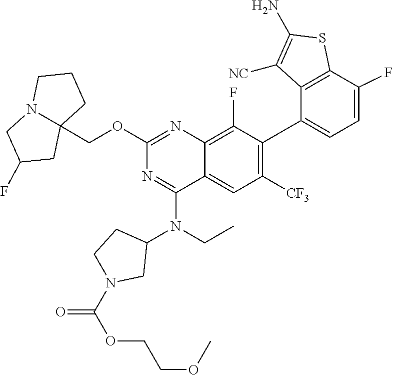 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OCCOC)C1] | |
| 686 | 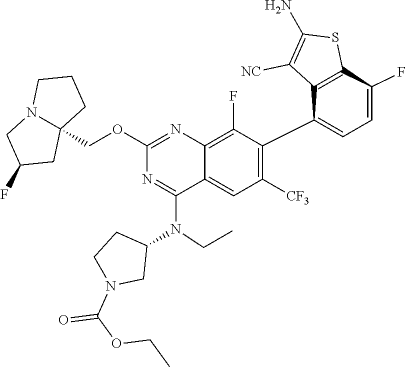 | LCMS m/z [M − 1]: 748.2 ¹H NMR (400 MHz, CD₃OD) δ = 8.23 (s, 1H), 7.25- 7.17 (m, 1H), 7.01 (t, J = 8.9, 1H), 5.38 (s, 0.5H), 5.25 (s, 0.5H), 5.08-4.95 (m, 1H), 4.29 (q, J = 10.7, 2H), 4.15 (q, J = 6.9, 2H), 3.98-3.69 (m, 4H), 3.63-3.52 (m, 1H), 3.44 (s, 2H), 3.25 (s, 2H), 3.09-2.99 (m, 1H), 2.51-2.10 (m, 5H), 2.05-1.85 (m, 3H), 1.45 (t, J = 6.8, 3H), 1.28 (s, 3H). |
| 686a | 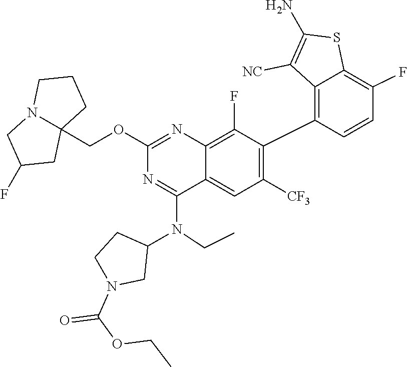 [SMILES: CCOC(=O)N1CCC(N(CC)c2nc(OCC34CCCN3CC(F)C4)nc3c(F)c(-c4ccc(F)c5sc(N)c(C#N)c45)c(C(F)(F)F)cc23)C1] | |
| 687 | 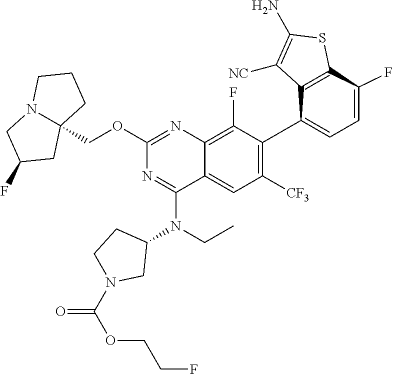 | LCMS m/z [M − 1]: 766.1. ¹H NMR (400 MHz, CD₃OD) δ 8.23 (s, 1H), 7.23-7.20 (m, 1H), 7.01 (t, J = 8.8 Hz, 1H), 5.37-5.24 (d, 1H), 5.04- 5.00 (m, 1H), 4.66 (s, 1H), 4.54 (s, 1H), 4.37-4.23 (m, 4H), 3.99-3.77 (m, 4H), 3.61-3.44 (m, 2H), 3.25- 3.21 (m, 2H), 3.02-3.01 (m, 1H), 2.49-1.87 (m, 8H), 1.45 (t, J = 7.2 Hz, 3H). |
| 687a | 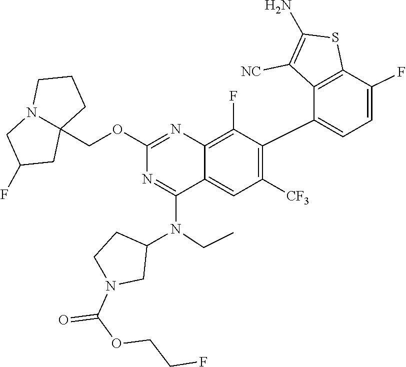 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OCCF)C1] | |
| 688 | 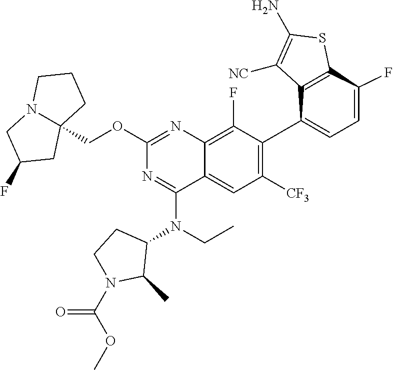 | LCMS m/z [M − 1]: 748.1 ¹H NMR (400 MHz, CD₃OD) δ 8.21 (s, 1H), 7.20-7.24 (m, 1H), 7.01 (t, 1H), 5.37 (s, 0.5H), 5.24 (s, 0.5H), 4.58-4.63 (m, 1H), 4.20-4.27 (m, 3H), 3.76-3.92 (m, 3H), 3.72 (s, 3H), 3.36-3.49 (m, 1H), 3.12-3.26 (m, 3H), 2.99-3.05 (m, 1H), 2.46-2.55 (m, 1H), 2.09-2.38 (m, 4H), 1.85-2.01 (m, 3H), 1.43 (t, 3H), 1.36 (d, 3H). |
| 688a | 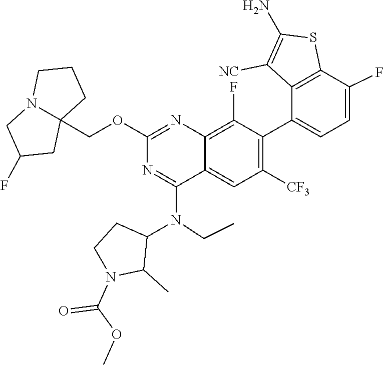 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC)C1C] | |
| 689 | 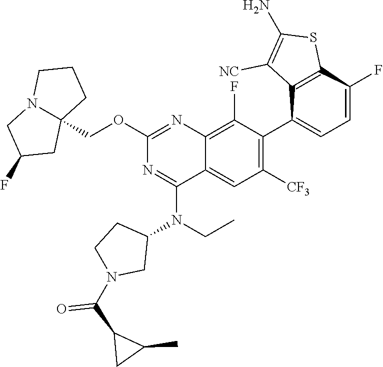 | LCMS m/z [M − 1]: 758.3 ¹H NMR (400 MHz, CD₃OD) δ 8.25 (s, 1H), 7.25-7.17 (m, 1H), 7.05-6.97 (m, 1H), 5.37 (s, 0.5H), 5.24 (s, 0.5H), 5.10-5.01 (m, 1H), 4.34-4.27 (m, 1H), 4.25-4.17 (m, 1H), 4.05-3.67 (m, 5H), 3.65-3.54 (m, 1H), 3.49- 3.33 (m, 2H), 3.28-3.20 (m, 2H), 3.07-2.99 (m, 1H), 2.55-1.80 (m, 10H), 1.50-1.36 (m, 3H), 1.33-1.26 (m, 1H), 1.11-1.06 (m, 2H), 0.96-0.87 (m, 2H). |
| 689a | 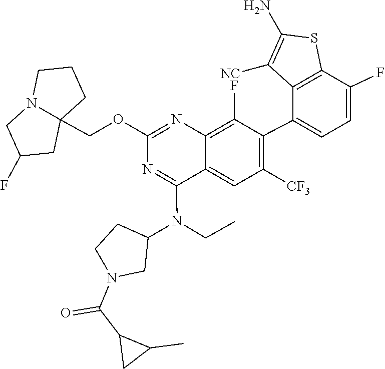 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CC2C)C1] | |
| 690 | 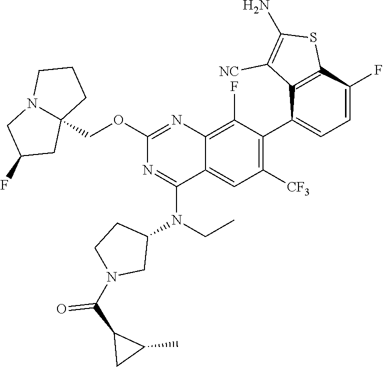 | LCMS m/z [M − 1]: 758.2 ¹H NMR (400 MHz, CD₃OD) δ 8.25 (d, 1H), 7.20-7.23 (m, 1H), 7.01 (t, 1H), 5.38 (s, 0.5H), 5.25 (s, 0.5H), 5.00-5.11 (m, 1H), 4.22-4.34 (m, 2H), 3.74-4.06 (m, 5H), 3.38-3.62 (m, 2H), 3.22-3.24 (m, 3H), 3.01-3.07 (m, 1H), 2.13-2.61 (m, 5H), 1.87-2.03 (m, 3H), 1.53- 1.60 (m, 1H), 1.43-1.49 (m, 3H), 1.08-1.17 (m, 4H), 0.64-0.70 (m, 1H). |
| 691 | 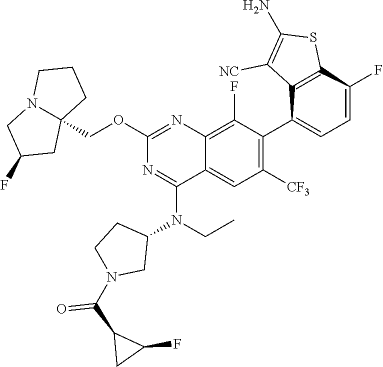 | LCMS m/z [M − 1]: 762.1 ¹H NMR (400 MHz, CD₃OD) δ 8.30-8.21 (m, 1H), 7.26-7.18 (m, 1H), 7.01 (t, 1H), 5.42 (s, 0.5H), 5.28 (s, 0.5H), 5.14-5.06 (m, 1H), 5.02-4.92 (m, 1H), 4.44-4.20 (m, 3H), 4.08-3.62 (m, 6H), 3.47-3.35 (m, 2H), 3.12- 3.05 (m, 1H), 2.60-2.42 (m, 2H), 2.39-2.17 (m, 3H), 2.12-1.90 (m, 4H), 1.76-1.68 (m, 1H), 1.56-1.38 (m, 3H), 1.13-1.06 (m, 1H). |
| 691a | 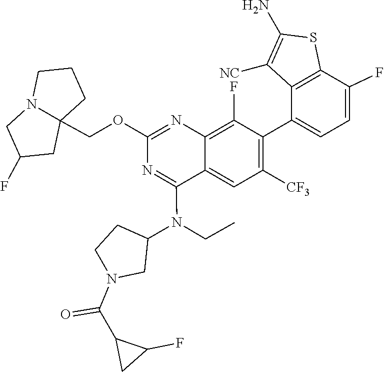 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CC2F)C1] | |
| 692 | 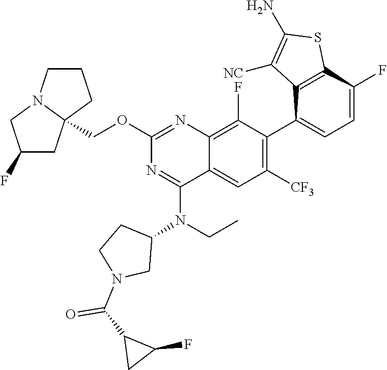 | LCMS m/z [M − 1]: 762.2 ¹H NMR (400 MHz, CD₃OD) δ 8.25 (d, 1H), 7.25-7.18 (m, 1H), 7.01 (t, 1H), 5.42-5.38 (m, 0.5H), 5.29-5.23 (m, 0.5H), 5.13-5.01 (m, 1H), 4.92-4.87 (m, 0.5H), 4.76-4.69 (m, 0.5H), 4.35-4.26 (m, 2H), 4.14-4.07 (m, 0.5H), 4.04-3.75 (m, 4H), 3.61-3.55 (m, 0.5H), 3.49- 3.32 (m, 2H), 3.29-3.21 (m, 2H), 3.10-3.01 (m, 1H), 2.54-1.84 (m, 9H), 1.51-1.43 (m, 3H), 1.33-1.23 (m, 2H). |
| 693 | 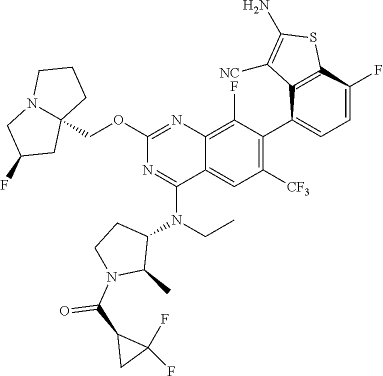 | LCMS m/z [M − 1]: 794.1 ¹H NMR (400 MHz, CD₃OD) δ 8.22 (s, 1H), 7.20-7.23 (m, 1H), 7.01 (t, 1H), 5.36 (s, 0.5H), 5.22 (s, 0.5H), 4.74-4.79 (m, 0.5H), 4.63-4.67 (m, 1H), 4.44-4.50 (m, 0.5H), 4.21-4.32 (m, 2H), 4.05-4.09 (m, 0.5H), 3.90- 3.95 (m, 1H), 3.76-3.85 (m, 2H), 3.46-3.53 (m, 0.5H), 3.12-3.25 (m, 3H), 2.80-3.01 (m, 2H), 2.31-2.63 (m, 3H), 2.08-2.25 (m, 3H), 1.95-2.00 (m, 2H), 1.69-1.88 (m, 2H), 1.45-1.49 (m, 3H), 1.35-1.38 (m, 3H). |
| 693a | 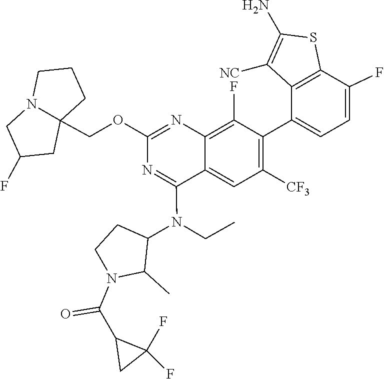 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CC2(F)F)C1C] | |
| 694 | 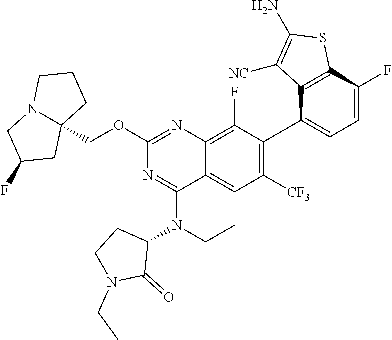 | LCMS m/z [M − 1]: 718.2 ¹H NMR (400 MHz, CD₃OD) δ = 8.28 (s, 1H), 7.23 (dd, J = 8.1, 5.1, 1H), 7.02 (t, J = 8.9, 1H), 5.43 (s, 0.5H), 5.29 (s, 0.5H), 4.70 (s, 1H), 4.28 (q, J = 11.2, 2H), 4.06-3.79 (m, 2H), 3.70-3.37 (m, 6H), 3.24-3.14 (m, 2H), 2.58- 1.93 (m, 8H), 1.57 (t, J = 7.0 Hz, 3H), 1.22 (t, J = 7.2 Hz, 3H). |
| 694a | 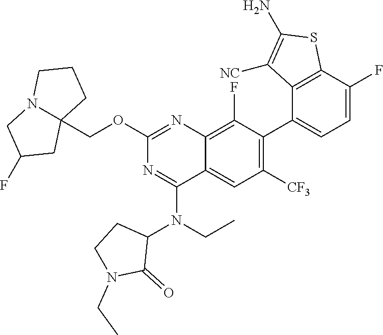 [SMILES: CCN1CCC(N(CC)c2nc(OCC34CCCN3CC(F)C4)nc3c(F)c(-c4ccc(F)c5sc(N)c(C#N)c45)c(C(F)(F)F)cc23)C1=O] | |
| 695 | 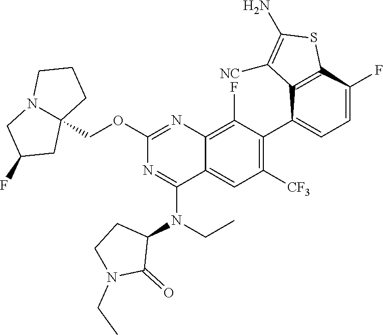 | LCMS m/z [M − 1]: 718.2 ¹H NMR (400 MHz, CD₃OD) δ 8.49 (brs, 0.38H), 8.31 (s, 1H), 7.23-7.16 (m, 1H), 7.00 (t, J = 8.8 Hz, 1H), 5.47 (s, 0.54H), 5.34 (s, 0.56H), 4.82-4.75 (m, 1H), 4.49- 4.42 (m, 1H), 4.32-4.25 (m, 1H), 4.08-3.95 (m, 1H), 3.91-3.81 (m, 1H), 3.66-3.49 (m, 6H), 3.27-3.12 (m, 2H), 2.60-2.35 (m, 4H), 2.30-1.96 (m, 4H), 1.54 (t, J = 7.0 Hz, 3H), 1.23 (t, J = 7.4 Hz, 3H). |
| 696 | 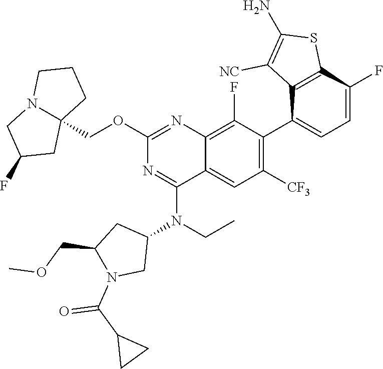 | LCMS m/z [M − 1]: 788.3 ¹H NMR (400 MHz, CD₃OD) δ 8.24-8.22 (d, J = 8 Hz, 1H), 7.31-7.13 (m, 1H), 7.07-6.89 (m, 1H), 5.52- 5.24 (m, 2H), 4.63-4.41 (m, 1H), 4.39-4.25 (m, 2H), 4.16-3.41 (m, 7H), 3.37-3.31 (m, 5H), 3.13-3.03 (m, 1H), 2.81-2.60 (m, 1H), 2.44-2.14 (m, 4H), 2.07- 1.79 (m, 4H), 1.48-1.38 (m, 3H), 0.96-0.77 (m, 4H). |
| 696a | 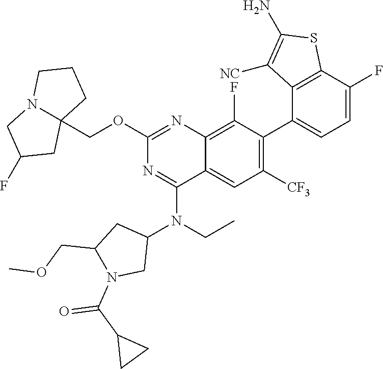 [SMILES: CCN(c1nc(OCC23CCCN2CC(F)C3)nc2c(F)c(-c3ccc(F)c4sc(N)c(C#N)c34)c(C(F)(F)F)cc12)C1CC(COC)N(C(=O)C2CC2)C1] | |
| 697 | 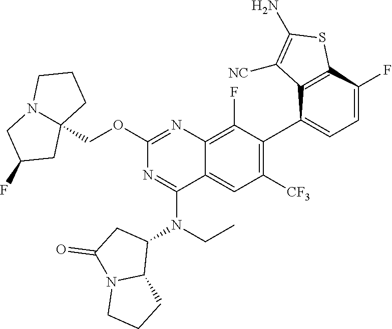 | LCMS m/z [M − 1]: 730.75 ¹H NMR (400 MHz, CD₃OD) δ 8.20 (s, 1H), 7.24-7.21 (m, 1H), 7.04-6.99 (m, 1H), 5.47 (s, 0.5H), 5.34 (s, 1H), 4.50-4.41 (m, 1H), 4.41-4.34 (m, 3H), 3.92-3.87 (m, 2H), 3.61-3.48 (m, 5H), 3.30-3.15 (m, 2H), 2.93-2.86 (m, 1H), 2.87-1.98 (m, 9H), 1.54-1.36 (m, 4H). |
| 697a | 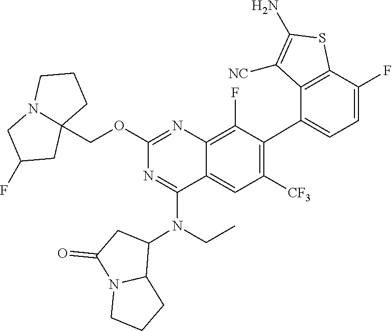 [SMILES: CCN(c1nc(OCC23CCCN2CC(F)C3)nc2c(F)c(-c3ccc(F)c4sc(N)c(C#N)c34)c(C(F)(F)F)cc12)C1CC(=O)N2CCCC12] | |
| 698 | 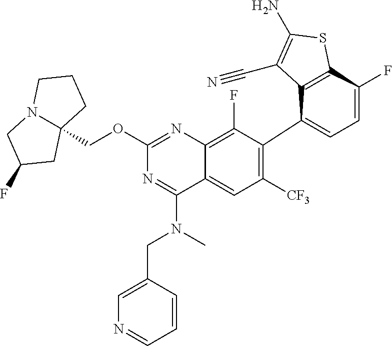 | LCMS m/z [M − 1]: 684.2 ¹H NMR (400 MHz, CD₃OD) δ = 8.64 (s, 1H), 8.51 (d, J = 4.7, 1H), 8.31 (s, 1H), 7.92 (d, J = 7.9, 1H), 7.48 (dd, J = 7.8, 4.9, 1H), 7.20 (dd, J = 8.2, 5.1 Hz, 1H), 7.00 (t, J = 8.9, 1H), 5.28 (d, J = 46.8, 1H), 5.21-5.11 (m, 2H), 4.19 (dd, J = 33.8, 10.6, 2H), 3.59 (s, 3H), 3.22 (dd, J = 24.9, 13.0, 3H), 3.06-2.92 (m, 1H), 2.31-1.82 (m, 6H). |
| 698a | 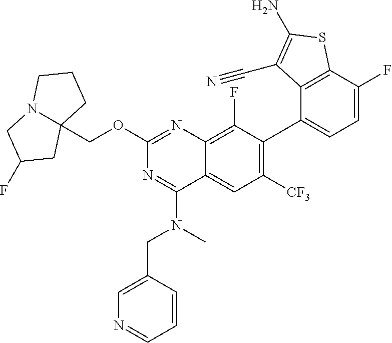 [SMILES: CN(Cc1cccnc1)c1nc(OCC23CCCN2CC(F)C3)nc2c(F)c(-c3ccc(F)c4sc(N)c(C#N)c34)c(C(F)(F)F)cc12] | |
| 699 | 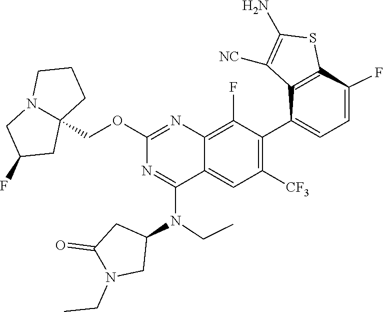 | LCMS m/z [M − 1]: 718.4 ¹H NMR (400 MHz, CD₃OD) δ 8.16 (s, 1H), 7.24-7.18 (m, 1H), 7.00 (t, 1H), 5.39 (m, 0.5H), 5.26 (m, 0.5H), 4.82-4.77 (m, 1H), 4.24-4.16 (m, 2H), 3.97-3.91 (m, 1H), 3.89-3.81 (m, 2H), 3.79-3.74 (m, 1H), 3.59-3.52 (m, 1H), 3.28-3.21 (m, 2H), 3.05-2.97 (m, 2H), 2.86- 2.79 (m, 1H), 2.41-1.86 (m, 8H), 1.51 (t, 3H), 1.19 (t, 3H). |
| 699a | 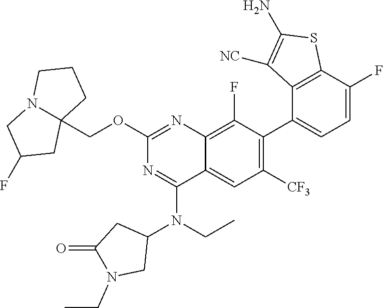 [SMILES: CCN1CC(N(CC)c2nc(OCC34CCCN3CC(F)C4)nc3c(F)c(-c4ccc(F)c5sc(N)c(C#N)c45)c(C(F)(F)F)cc23)CC1=O] | |
| 700 | 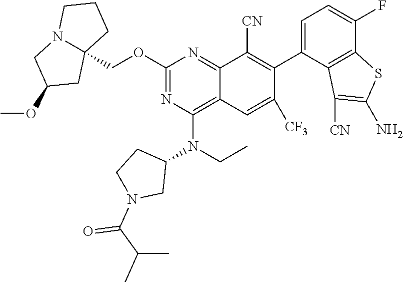 | LCMS m/z [M − 1]: 758.3 ¹H NMR (400 MHz, CD₃OD) δ 8.27 (m, 1H), 7.20-7.24 (m, 1H), 7.02 (t, J = 8.9 Hz, 1H), 5.06-5.10 (m, 1H), 4.36-4.52 (m, 2H), 3.49-4.18 (m, 9H), 3.34 (s, 3H), 2.75-2.89 (m, 1H), 2.34-2.54 (m, 3H), 1.97-2.17 (m, 5H) 1.45-1.48 (m, 3H), 1.12-1.16 (m, 6H). |
| 700a | 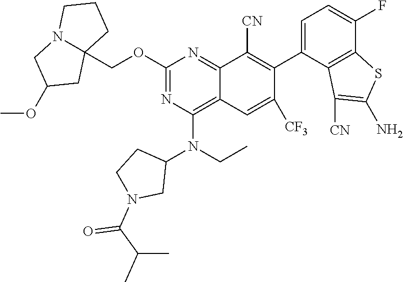 [SMILES: CCN(c1nc(OCC23CCCN2CC(OC)C3)nc2c(C#N)c(-c3ccc(F)c4sc(N)c(C#N)c34)c(C(F)(F)F)cc12)C1CCN(C(=O)C(C)C)C1] | |
| 701 | 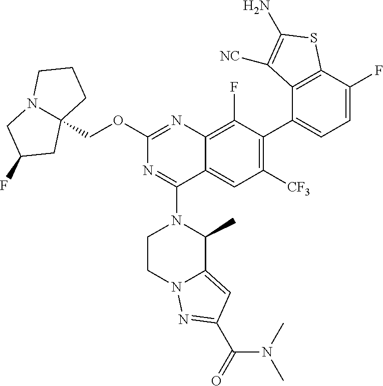 | LCMS m/z [M − 1]: 770.2 ¹H NMR (400 MHz, CD₃OD) δ 8.22-8.51 (m, 1H), 7.21-7.25 (m, 1H), 7.02 (t, J = 9.52 Hz, 1H), 6.52 (s, 1H), 5.86-5.95 (m, 1H), 5.36 (d, J = 54.48 Hz, 1H), 4.09-4.65 (m, 6H), 3.08-3.48 (m, 8H), 2.03-2.47 (m, 6H), 1.89-1.94 (m, 3H). |
| 701a | 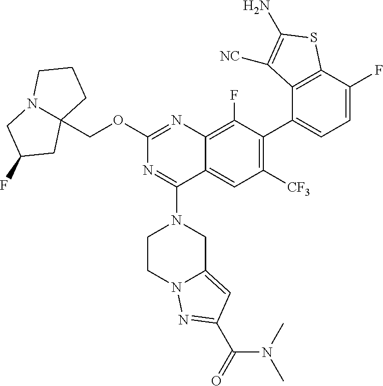 [SMILES: CN(C)C(=O)c1cc2n(n1)CCN(c1nc(OCC34CCCN3C[C@H](F)C4)nc3c(F)c(-c4ccc(F)c5sc(N)c(C#N)c45)c(C(F)(F)F)cc13)C2] | |
| 702 | 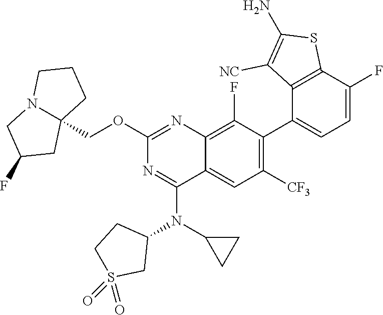 [SMILES: N#Cc1c(N)sc2c(F)ccc(-c3c(C(F)(F)F)cc4c(N(C5CC5)[C@H]5CCS(=O)(=O)C5)nc(OC[C@@]56CCCN5C[C@H](F)C6)nc4c3F)c12] | LCMS m/z [M − 1]: 737.17 ¹H NMR (400 MHz, CD₃OD) δ 8.74 (s, 1H), 7.24-7.18 (m, 1H), 7.04-6.96 (m, 1H), 5.38 (s, 0.5H), 5.24 (s, 0.5H), 5.01-4.91 (m, 1H), 4.35-4.21 (m, 2H), 3.84-3.53 (m, 4H), 3.27-3.10 (m, 3H), 3.07-2.97 (m, 1H), 2.96- 2.86 (m, 1H), 2.82-2.71 (m, 1H), 2.43-1.79 (m, 7H), 1.12-1.01 (m, 2H), 0.91-0.75 (m, 2H). |
| 702a | 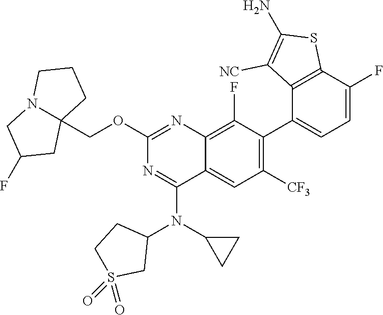 [SMILES: N#Cc1c(N)sc2c(F)ccc(-c3c(C(F)(F)F)cc4c(N(C5CC5)C5CCS(=O)(=O)C5)nc(OCC56CCCN5CC(F)C6)nc4c3F)c12] | |
| 703 | 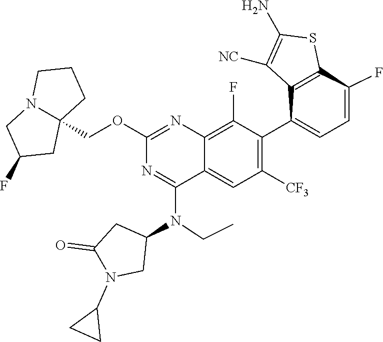 | LCMS m/z [M − 1]: 730.1 ¹H NMR (400 MHz, CD₃OD) δ 8.15 (s, 1H), 7.20 (t, 1H), 7.00 (t, 1H), 5.40 (s, 0.5H), 5.27 (s, 0.5H), 4.68- 4.69 (m, 1H), 4.19-4.27 (m, 2H), 3.70-3.86 (m, 4H), 3.34-3.44 (m, 1H), 3.19-3.20 (m, 2H), 2.95-3.04 (m, 2H), 2.78-2.85 (m, 2H), 2.15-2.38 (m, 3H), 1.91-2.02 (m, 3H), 1.49-1.52 (m, 3H), 0.74-0.84 (m, 4H). |
| 703a | 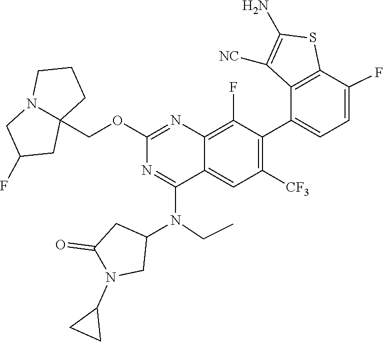 [SMILES: CCN(c1nc(OCC23CCCN2CC(F)C3)nc2c(F)c(-c3ccc(F)c4sc(N)c(C#N)c34)c(C(F)(F)F)cc12)C1CC(=O)N(C2CC2)C1] | |
| 704 | 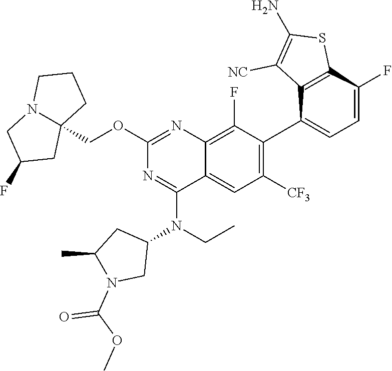 | LCMS m/z [M − 1]: 748.1 ¹H NMR (400 MHz, CD₃OD) δ 8.45 (s, 1H), 8.26 (s, 1H), 7.22-7.21 (m, 1H), 7.04-6.99 (m, 1H), 5.53 (s, 0.5H), 5.40 (s, 0.5H), 5.19 (m, 1H), 4.51-4.45 (m, 2H), 4.22 (m, 1H), 3.67-3.58 (m, 10H), 2.65-2.05 (m, 9H), 1.46-1.44 (m, 3H), 1.28-1.27 (m, 3H). |
| 704a | 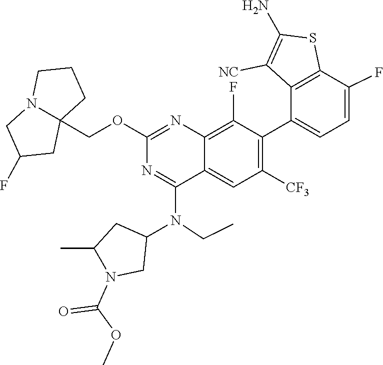 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)OC)C1] | |
| 705 | 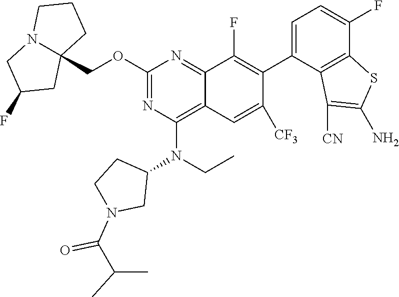 [SMILES: CCN(c1nc(OC[C@]23CCCN2C[C@H](F)C3)nc2c(F)c(-c3ccc(F)c4sc(N)c(C#N)c34)c(C(F)(F)F)cc12)[C@H]1CCN(C(=O)C(C)C)C1] | LCMS m/z [M − 1]: 746.4 ¹H NMR (400 MHz, CD₃OD) δ 8.17-8.30 (m, 1H), 7.18-7.30 (m, 1H), 7.01 (t, J = 8.9 Hz, 1H), 5.34 (d, J = 53.1 Hz, 1H), 5.03-5.20 (m, 1H), 4.52-4.68 (m, 1H), 4.32-4.47 (m, 2H), 3.78-4.06 (m, 3H), 3.39-3.76 (m, 3H), 2.73-3.12 (m, 4H), 2.28-2.31 (m, 3H), 2.09-2.19 (m, 1H), 1.89-2.09 (m, 3H), 1.80-1.89 (m, 1H), 1.28- 1.51 (m, 3H), 1.03-1.20 (m, 6H). |
| 705a | 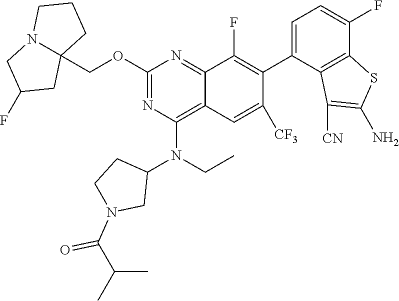 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C(C)C)C1] | |
| 706 | 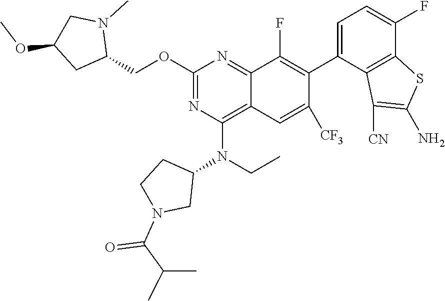 [SMILES: CCN(c1nc(OC[C@@H]2C[C@@H](OC)CN2C)nc2c(F)c(-c3ccc(F)c4sc(N)c(C#N)c34)c(C(F)(F)F)cc12)[C@H]1CCN(C(=O)C(C)C)C1] | LCMS m/z [M − 1]: 732.3 ¹H NMR (400 MHz, CD₃OD) δ 8.30 (s, 1H), 7.27- 7.19 (m, 1H), 7.03 (t, J = 8.9 Hz, 1H), 5.15-5.01 (m, 1H), 5.01-4.88 (m, 1H), 4.73-4.58 (m, 1H), 4.17 (s, 1H), 4.10 (d, J = 7.5 Hz, 1H), 3.92 (dddd, J = 41.8, 36.0, 20.2, 8.8 Hz, 5H), 3.72-3.50 (m, 1H), 3.43 (d, J = 10.5 Hz, 1H), 3.39 (s, 3H), 3.13 (s, 3H), 2.82 (dp, J = 25.9, 6.8 Hz, 1H), 2.61-2.34 (m, 3H), 2.17 (d, J = 14.8 Hz, 1H), 1.53-1.43 (m, 3H), 1.17-1.10 (m, 6H). |
| 706a | 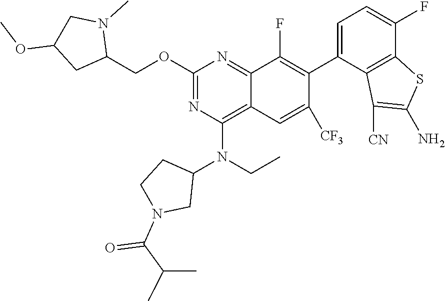 [SMILES: CCN(c1nc(OCC2CC(OC)CN2C)nc2c(F)c(-c3ccc(F)c4sc(N)c(C#N)c34)c(C(F)(F)F)cc12)C1CCN(C(=O)C(C)C)C1] | |
| 707 | 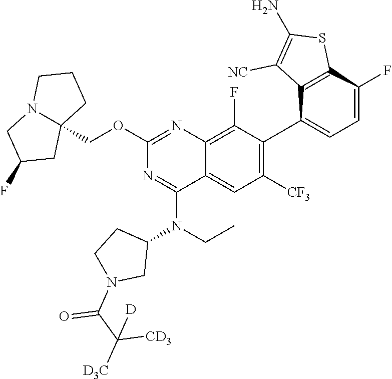 | LCMS m/z [M − 1]: 753.2 ¹H NMR (400 MHz, CD₃OD) δ 8.24 (d, 1H), 7.20-7.23 (m, 1H), 7.01 (t, 1H), 5.37 (s, 0.5H), 5.23 (s, 0.5H), 5.01-5.10 (m, 1H), 3.80-4.32 (m, 6H), 3.55-3.64 (m, 2H), 3.38-3.47 (m, 1H), 3.21-3.26 (m, 2H), 3.01-3.02 (m, 1H), 2.10-2.52 (m, 5H), 1.88-2.01 (m, 3H), 1.42- 1.48 (m, 3H). |
| 707a | 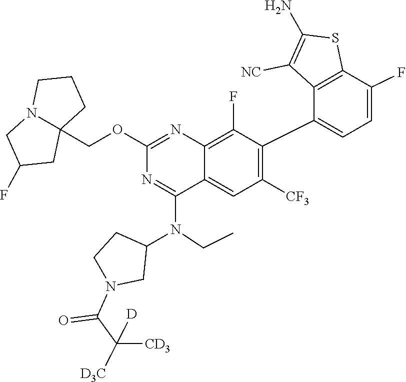 [SMILES: [2H]C([2H])([2H])C([2H])(C(=O)N1CCC(N(CC)c2nc(OCC34CCCN3CC(F)C4)nc3c(F)c(-c4ccc(F)c5sc(N)c(C#N)c45)c(C(F)(F)F)cc23)C1)C([2H])([2H])[2H]] | |
| 708 | 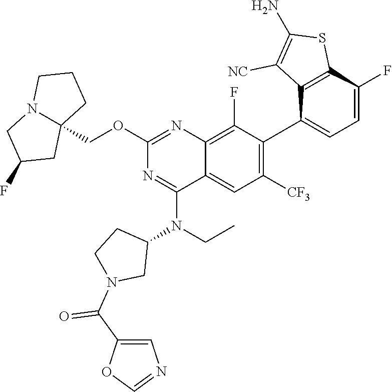 | LCMS m/z [M − 1]: 771.1 ¹H NMR (400 MHz, CD₃OD) δ 8.398 (d, 1H), 8.27 (s, 1H), 7.804 (d, 1H), 7.214 (t, 1H), 7.012 (t, 1H), 5.337 (s, 0.5H), 5.208 (s, 0.5H), 5.115-5.013 (m, 1H), 4.470- 4.348 (m, 1H), 4.304-4.155 (m, 3H), 4.077-3.855 (m, 4H), 3.737-3.610 (m, 1H), 3.222 (s, 2H), 2.989 (s, 1H), 2.633-2.436 (m, 2H), 2.115-2.050 (m, 1H), 2.298-2.159 (m, 2H), 1.998-1.911 (m, 2H), 1.831 (s, 1H), 1.550- 1.457 (m, 3H). |
| 708a | 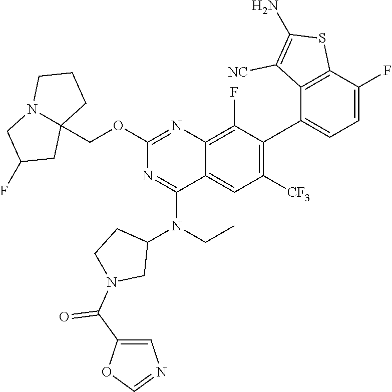 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2cnco2)C1] | |
| 709 | 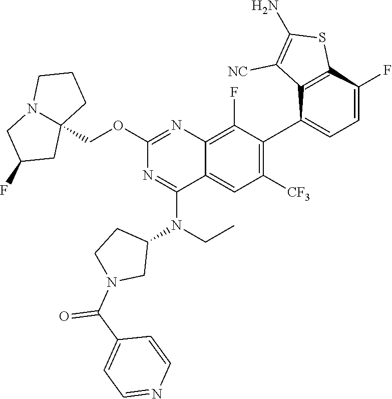 | LCMS m/z [M − 1]: 781.2 ¹H NMR (400 MHz, CD₃OD) δ 8.63-8.75 (m, 2H), 8.45 (s, 1H), 8.18-8.35 (m, 1H), 7.53-7.64 (m, 2H), 7.16-7.27 (m, 1H), 6.97-7.06 (m, 1H), 5.46-5.56 (m, 0.5 H), 5.32- 5.42 (m, 0.5H), 4.97-5.19 (m, 1H), 4.12-4.64 (m, 3H), 3.56-4.07 (m, 8H), 3.25-3.29 (m, 1H), 1.93-2.62 (m, 8H), 1.39-1.54 (m, 3H). |
| 709a | 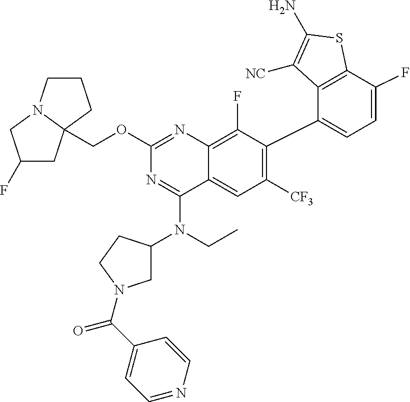 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2ccncc2)C1] | |
| 710 | 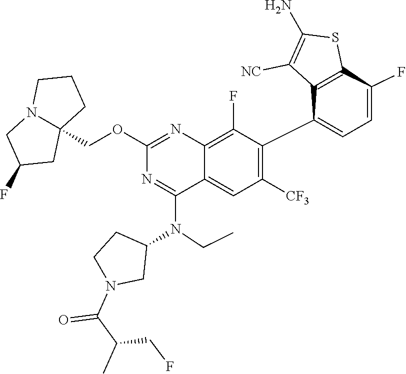 | LCMS m/z [M − 1]: 764.2 ¹H NMR (400 MHz, CD₃OD) δ = 8.28 (d, J = 10.0 Hz, 1H), 7.24-7.20 (m, 1H), 7.02 (t, J = 8.9 Hz, 1H), 5.51 (s, 0.5H), 5.38 (s, 0.5H), 5.07-4.99 (m, 1H), 4.68-4.34 (m, 4H), 4.11 (d, J = 8.1, 1H), 3.96-3.88 (m, 3H), 3.75-3.45 (m, 5H), 3.13 (s, 1H), 2.56-1.93 (m, 9H), 1.51-1.45 (m, 3H), 1.17-1.09 (m, 3H). |
| 710a | 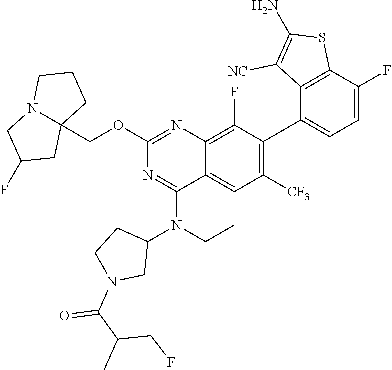 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C(C)CF)C1] | |
| 711 | 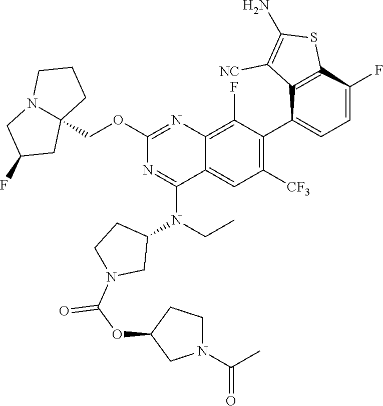 | LCMS m/z [M + 1]: 831.2 ¹H NMR (400 MHz, CD₃OD) δ 8.22 (s, 1H), 7.21 (m, 1H), 7.01 (m, 1H), 5.41-5.23 (m, 2H), 5.02 (s, 1H), 4.39-4.17 (m, 2H), 3.97-3.80 (m, 3H), 3.76-3.54 (m, 5H), 3.44 (m, 2H), 3.24 (m, 3H), 3.09-2.96 (m, 1H), 2.44 (m, 1H), 2.39-2.19 (m, 4H), 2.13 (m, 3H), 2.07-1.97 (m, 4H), 1.90 (m, 1H), 1.44 (m, 3H). |
| 711a | 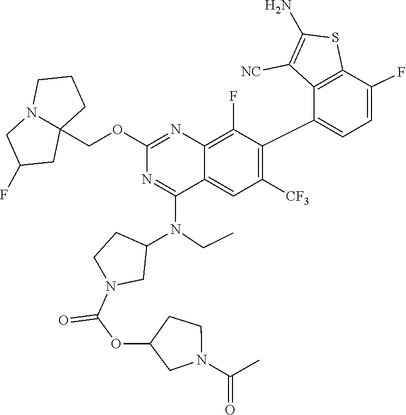 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2CCN(C(C)=O)C2)C1] | |
| 712 | 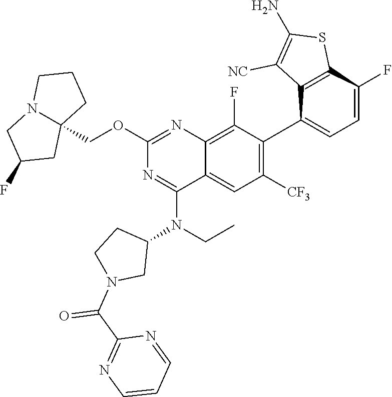 | LCMS m/z [M − 1]: 782.2 ¹H NMR (400 MHz, CD₃OD) δ = 8.91 (dd, J = 14.9, 5.0, 2H), 8.27 (s, 0.5H), 8.20 (s, 0.5H), 7.58 (dt, J = 15.1 Hz, 5.0 Hz, 1H), 7.21 (dd, J = 15.9 Hz, 8.8 Hz, 1H), 7.05 (m, 1H), 5.40 (s, 0.5H), 5.26 (s, 0.5H), 5.05 (dd, J = 22.5 Hz, 8.4 Hz, 1H), 4.40-4.32 (m, 1H), 4.21-4.08 (m, 2H), 3.92 (d, J = 7.6 Hz, 4H), 3.71 (d, J = 9.3 Hz, 1H), 3.51- 3.40 (m, 1H), 3.07 (s, 1H), 2.62-2.02 (m, 9H), 1.47 (t, J = 8.0 Hz, 3H). |
| 712a | 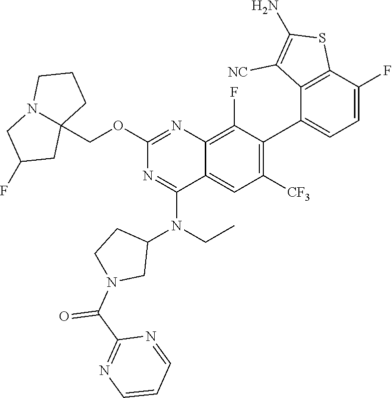 | |
| 713 | 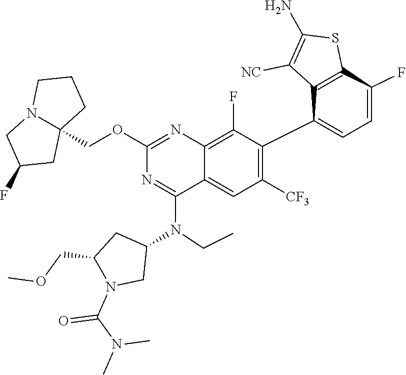 | LCMS m/z [M − 1]: 791.3 ¹H NMR (400 MHz, CD₃OD) δ 8.24 (s, 1H), 7.23-7.19 (m, 1H), 7.03-6.98 (m, 1H), 5.38 (s, 0.5H), 5.24 (s, 0.5H), 4.89-4.86 (m, 1H), 4.35-4.22 (m, 3H), 3.91-3.80 (m, 3H), 3.61-3.46 (m, 3H), 3.39 (s, 3H), 3.26-3.23 (m, 3H), 3.04-3.03 (m, 1H), 2.87 (s, 6H), 2.47-2.44 (m, 1H), 2.34-2.09 (m, 4H), 2.03-1.89 (m, 3H), 1.47-1.44 (m, 3H). |
| 713a | 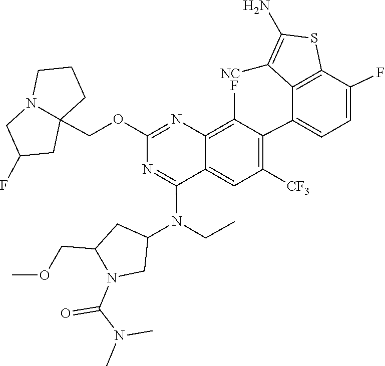 [SMILES: CCN(c1nc(OCC23CCCN2CC(F)C3)nc2c(F)c(-c3ccc(F)c4sc(N)c(C#N)c34)c(C(F)(F)F)cc12)C1CC(COC)N(C(=O)N(C)C)C1] | |
| 714 | 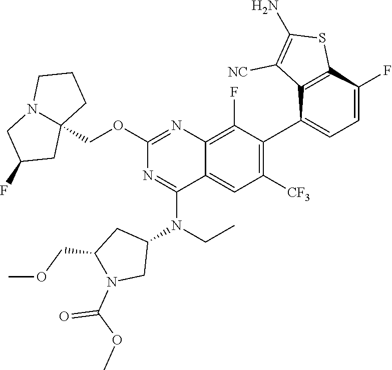 | LCMS m/z [M − 1]: 778.2 ¹H NMR (400 MHz, CD₃OD) δ 8.23 (s, 1H), 7.23-7.19 (m, 1H), 7.03-6.98 (m, 1H), 5.38 (s, 0.5H), 5.25 (s, 0.5H), 4.96-4.94 (m, 1H), 4.38-4.29 (m, 2H), 4.20-4.04 (m, 2H), 3.89-3.87 (m, 2H), 3.71-3.59 (m, 5H), 3.42- 3.40 (m, 4H), 3.26-3.24 (m, 3H), 3.06-3.04 (m, 1H), 2.55-1.95 (m, 8H), 1.42-1.39 (m, 3H). |
| 714a | 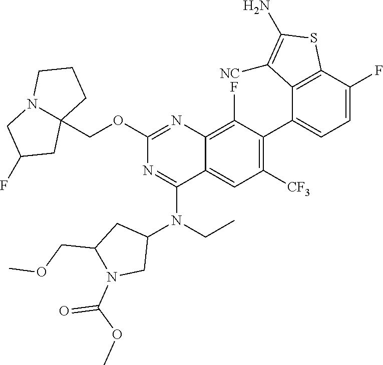 [SMILES: CCN(c1nc(OCC23CCCN2CC(F)C3)nc2c(F)c(-c3ccc(F)c4sc(N)c(C#N)c34)c(C(F)(F)F)cc12)C1CC(COC)N(C(=O)OC)C1] | |
| 715 | 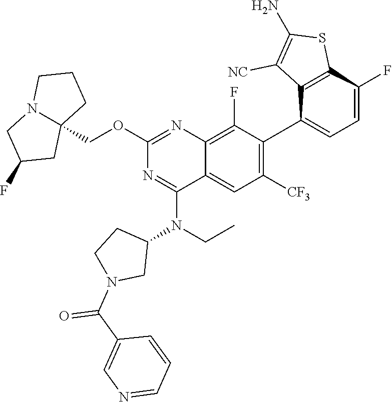 | LCMS m/z [M − 1]: 781.2 ¹H NMR (400 MHz, CD₃OD) δ 8.78 (s, 1H), 8.69- 8.8.67 (m, 1H), 8.32-8.26 (m, 2H), 8.06-8.03 (m, 1H), 7.57-7.53 (m, 1H), 7.22-7.19 (m, 1H), 7.01-6.99 (m, 1H), 5.59 (m, 0.5H), 5.46 (m, 0.5H), 5.12 (m, 0.5H), 5.04 (m, 0.5H), 4.70-4.61 (m, 2H), 4.43-4.18 (m, 1H), 4.18-3.71 (m, 7H), 3.30-2.41 (m, 1H), 2.69-2.09 (m, 9H), 1.49-1.51 (m, 3H). |
| 715a | 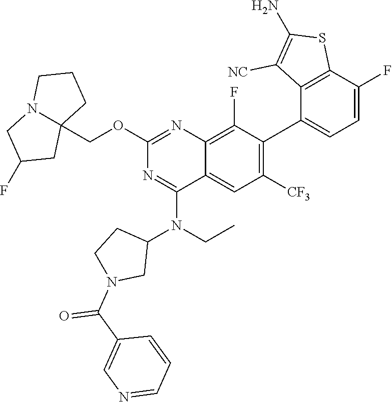 | |
| 716 | 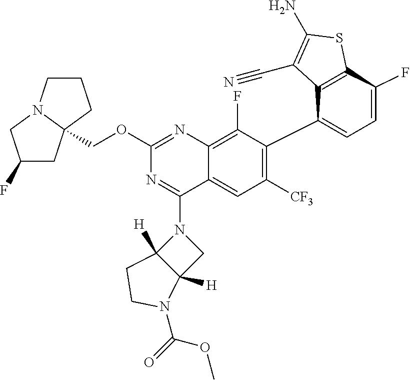 | LCMS m/z [M − 1]: 718.1 ¹H NMR (400 MHz, CD₃OD) δ 8.04 (s, 1H), 7.20-7.17 (m, 1H) 7.02-6.98 (m, 1H), 5.56 (s, 1H), 5.31 (s, 0.5H), 5.22 (S, 1H), 4.79 (m, 1H), 4.27 (s, 1H), 4.27-4.21 (m, 2H), 4.01-4.08 (m, 1H), 4.08-3.79 (m, 4H), 3.3-3.19 (m, 3H), 3.19-3.01 (m, 1H), 2.54-2.26 (m, 1H), 2.26- 1.87 (m, 8H). |
| 716a | 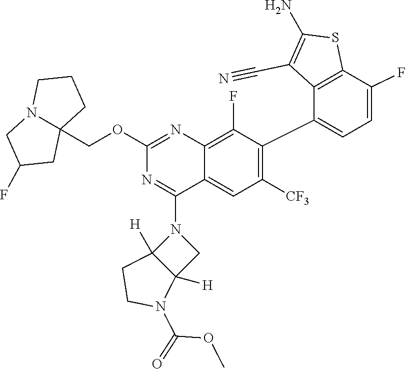 [SMILES: COC(=O)N1CCC2C1CN2c1nc(OCC23CCCN2CC(F)C3)nc2c(F)c(-c3ccc(F)c4sc(N)c(C#N)c34)c(C(F)(F)F)cc12] | |
| 717 | 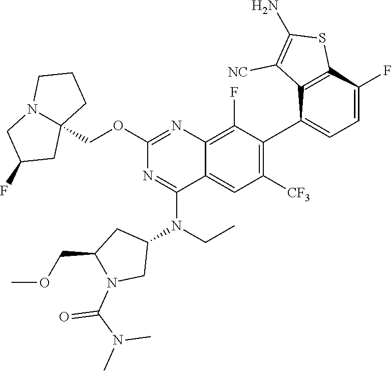 | LCMS m/z [M − 1]: 791.3 ¹H NMR (400 MHz, CD₃OD) δ 8.20 (s, 1H), 7.26-7.13 (m, 1H), 7.05-6.91 (m, 1H), 5.48-5.16 (m, 2H), 4.63- 4.54 (m, 1H), 4.39-4.36 (d, J = 12 Hz, 1H), 4.29-4.26 (d, J = 12 Hz, 1H), 4.03-3.92 (m, 1H), 3.86-3.67 (m, 2H), 3.58-3.52 (m, 1H), 3.42-3.41 (d, J = 4 Hz, 2H),), 3.32-3.27 (m, 6H), 3.12-3.03 (m, 1H), 2.87 (s, 6H), 2.53-2.15 (m, 5H), 2.08-1.86 (m, 3H), 1.40-1.32 (m, 3H). |
| 718 | 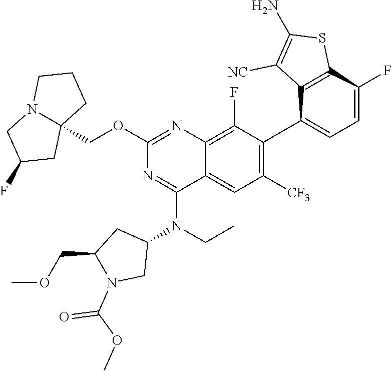 | LCMS m/z [M − 1]: 778.2 ¹H NMR (400 MHz, CD₃OD) δ 8.20 (s, 1H), 7.26-7.16 (m, 1H), 7.06-6.96 (m, 1H), 5.41-5.24 (m, 2H), 4.36- 4.24 (m, 2H), 4.23-4.13 (m, 1H), 3.93-3.83 (m, 1H), 3.80-3.69 (m, 5H), 3.68-3.53 (m, 2H), 3.49-3.34 (m, 2H), 3.33 (s, 3H), 3.28-3.25 (m, 1H), 3.10-3.00 (m, 1H), 2.73-2.53 (m, 1H), 2.47-1.76 (m, 8H), 1.43- 1.37 (m, 3H). |
| 719 | 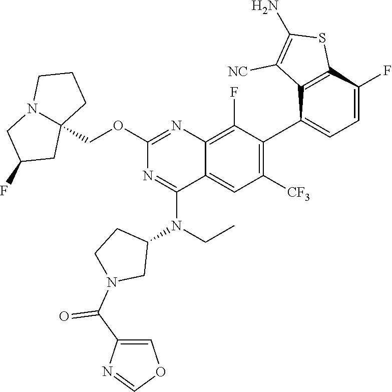 | LCMS m/z [M − 1]: 771.3 ¹H NMR (400 MHz, CD₃OD) δ 8.47 (s, 1H), 8.31-8.18 (m, 2H), 7.24-7.18 (m, 1H), 7.01 (t, 1H), 5.34 (s, 0.5H), 5.20 (s, 0.5H), 5.09-5.01 (m, 1H), 4.63-4.55 (m, 1H), 4.49-4.39 (m, 1H), 4.30-4.12 (m, 3H), 4.04-3.87 (m, 3H), 3.68-3.59 (m, 1H), 3.23-3.18 (m, 2H), 3.03-2.96 (m, 1H), 2.55-2.39 (m, 2H), 2.33-2.10 (m, 3H), 2.01- 1.84 (m, 3H), 1.53-1.42 (m, 3H). |
| 719a | 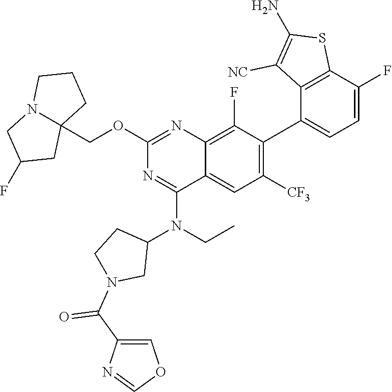 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2cocn2)C1] | |
| 720 | 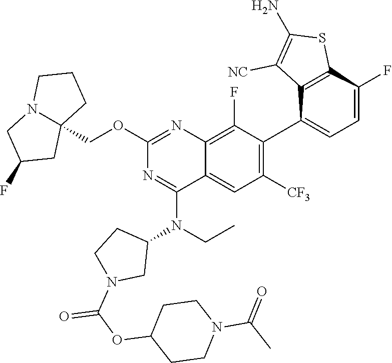 | LCMS m/z [M − 1]: 845.3 ¹H NMR (400 MHz, CD₃OD) δ 8.23 (s, 1H), 7.24-7.17 (m, 1H), 7.01 (t, 1H), 5.37 (s, 0.5H), 5.24 (s, 0.5H), 5.06-4.98 (m, 1H), 4.94-4.89 (m, 1H), 4.34-4.20 (m, 2H), 3.99-3.67 (m, 6H), 3.60-3.42 (m, 4H), 3.28-3.19 (m, 3H), 3.06-2.97 (m, 1H), 2.53-2.22 (m, 4H), 2.20- 2.06 (m, 4H), 2.04-1.82 (m, 5H), 1.76-1.61 (m, 2H), 1.49-1.36 (m, 3H). |
| 720a | 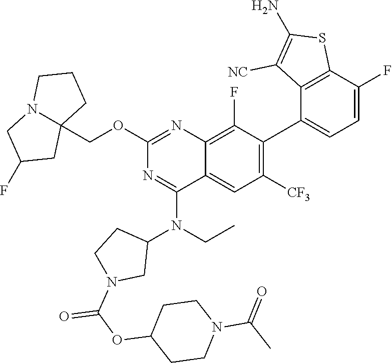 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2CCN(C(C)=O)CC2)C1] | |
| 721 | 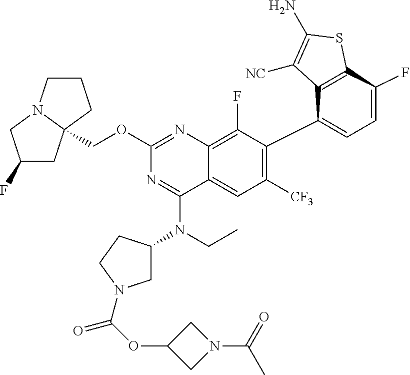 | LCMS m/z [M − 1]: 817.2 ¹H NMR (400 MHz, CD₃OD) δ 8.23 (s, 1H), 7.24-7.18 (m, 1H), 7.01 (t, 1H), 5.37 (s, 0.5H), 5.23 (s, 0.5H), 5.174 (s, 1H), 5.05-4.99 (m, 1H), 4.55-4.49 (m, 1H), 4.31-4.18 (m, 3H), 4.01-3.81 (m, 4H), 3.77-3.40 (m, 5H), 3.25-3.18 (m, 2H), 3.05-2.98 (m, 1H), 2.51-2.30 (m, 3H), 2.28-2.08 (m, 3H), 2.03-1.93 (m, 2H), 1.92-1.83 (m, 3H), 1.52-1.36 (m, 3H). |
| 721a | 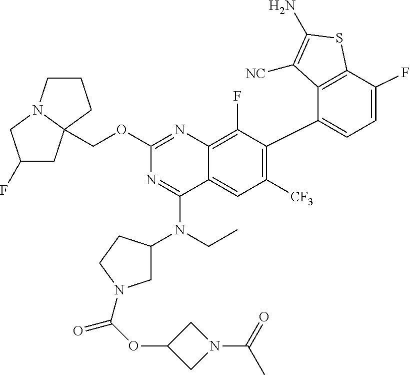 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2CN(C(C)=O)C2)C1] | |
| 722 | 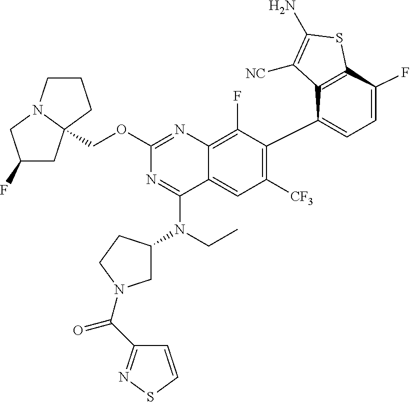 | LCMS m/z [M − 1]: 787.2 ¹H NMR (400 MHz, CD₃OD) δ 8.97-9.00 (m, 1H), 8.27 (d, 1H), 7.77-7.79 (m, 1H), 7.20-7.23 (m, 1H), 6.99- 7.03 (m, 1H), 5.37-5.40 (m, 0.5H), 5.24-5.27 (m, 0.5H), 5.05-5.09 (m, 1H), 4.56-4.61 (m, 1H), 4.16-4.39 (m, 3H), 3.88-3.98 (m, 3H), 3.67-3.70 (m, 1H), 3.35-3.47 (m, 3H), 3.08-3.12 (m, 1H), 2.53-2.60 (m, 1H), 2.18- 2.44 (m, 3H), 2.10-2.18 (m, 1H), 2.03 (s, 2H), 1.90-1.93 (m, 1H), 1.50 (t, 3H). |
| 722a | 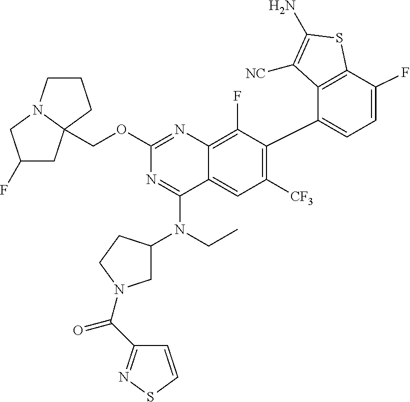 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2ccsn2)C1] | |
| 723 | 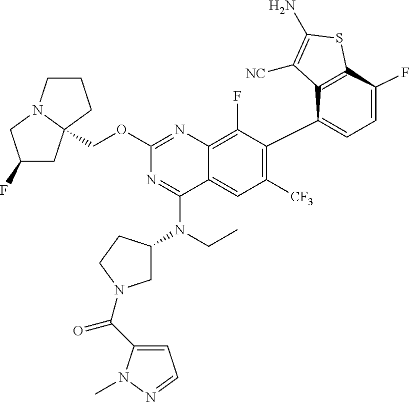 | LCMS m/z [M − 1]: 784.20 ¹H NMR (400 MHz, CD₃OD) δ 8.25 (d, 1H), 7.49 (d, 1H), 7.24-7.18 (m, 1H), 7.01 (t, 1H), 6.68 (d, 1H), 5.34 (d, 0.5H), 5.21 (d, 0.5H), 5.14-5.08 (m, 0.5H), 5.03-4.99 (m, 0.5H), 4.40-4.24 (m, 2H), 5.25-4.09 (m, 2H), 4.05- 3.98 (m, 3H), 3.97-3.84 (m, 3H), 3.81-3.69 (m, 2H), 3.25-3.14 (m, 2H), 3.03 (s, 1H), 2.54-2.40 (m, 2H), 2.31-2.11 (m, 3H), 2.02-1.95 (m, 2H), 1.90-1.81 (m, 1H), 1.51-1.42 (m, 3H). |
| 723a | 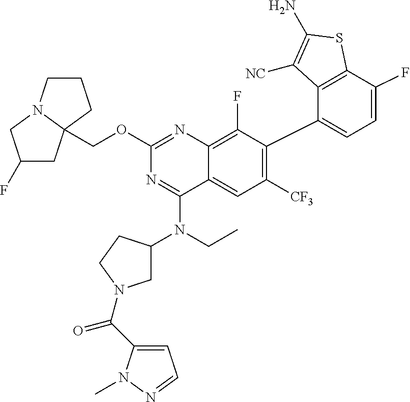 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2ccnn2C)C1] | |
| 724 | 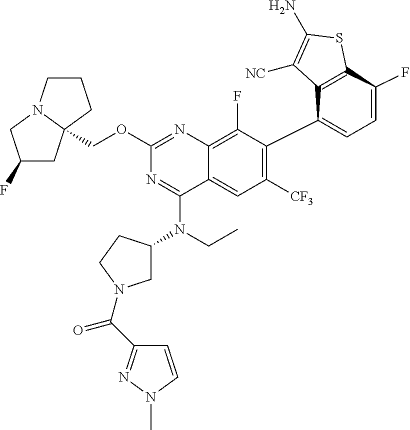 | LCMS m/z [M − 1]: 784.2 ¹H NMR (400 MHz, CD₃OD) δ 8.25 (s, 1H), 7.61-7.64 (m, 1H), 7.21 (t, 1H), 7.01 (t, 1H), 6.75 (t, 1H), 5.30 (d, 0.5H), 5.17 (d, 0.5H), 5.01-5.09 (m, 1H), 4.45-4.60 (m, 1H), 4.13-4.26 (m, 3H), 4.00-4.05 (m, 1H), 3.92-3.96 (m, 4H), 3.84-3.89 (m, 1H), 3.47-3.68 (m, 1H), 3.10- 3.17 (m, 3H), 2.93-2.99 (m, 1H), 2.12-2.63 (m, 4H), 2.03-2.07 (m, 1H), 1.76-1.93 (m, 3H), 1.44 (t, 3H). |
| 724a | 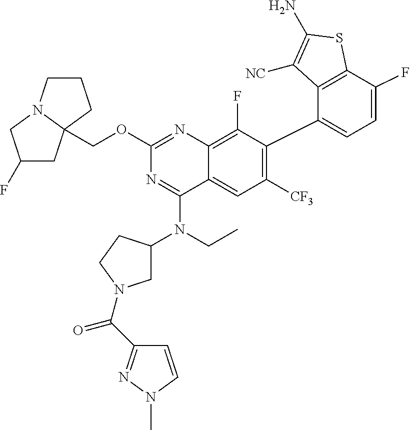 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2ccn(C)n2)C1] | |
| 725 | 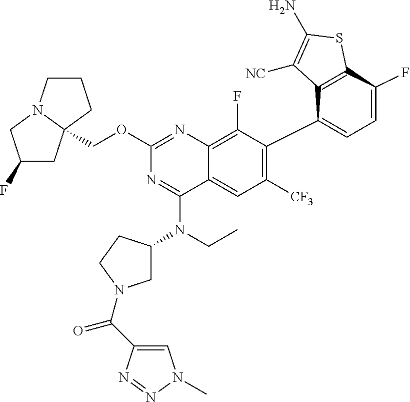 | LCMS m/z [M − 1]: 785.2 ¹H NMR (400 MHz, CD₃OD) δ 8.39 (d, 1H), 8.28 (d, 1H), 7.20-7.24 (m, 1H), 7.01 (t, 1H), 5.46 (s, 0.5H), 5.33 (s, 0.5H), 5.03-5.08 (m, 1H), 4.68-4.73 (m, 1H), 4.36-4.52 (m, 3H), 4.13-4.16 (m, 4H), 3.88-3.98 (m, 2H), 3.40-3.68 (m, 4H), 3.16-3.23 (m, 1H), 2.33-2.62 (m, 4H), 2.17-2.23 (m, 1H), 1.96-2.13 (m, 3H), 1.49- 1.53 (m, 3H). |
| 725a | 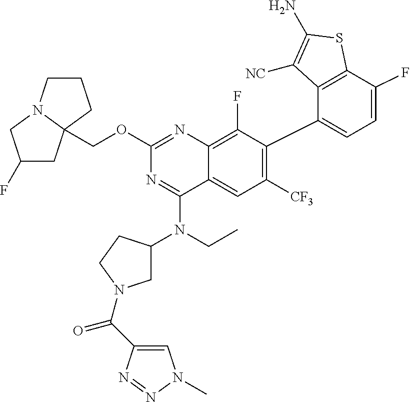 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2cn(C)nn2)C1] | |
| 726 | 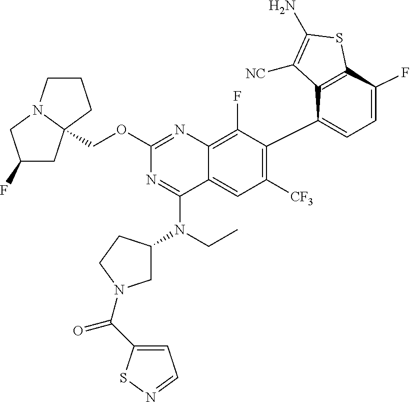 | LCMS m/z [M − 1]: 787.1 ¹H NMR (400 MHz, CD₃OD) δ 8.57 (d, 1H), 8.26 (s, 1H), 7.77 (d, 1H), 7.22 (s, 1H), 7.01 (t, 1H), 5.35-5.28 (m, 0.5H), 5.20-5.14 (m, 0.5H), 5.12-5.04 (m, 1H), 4.37-4.03 (m, 4H), 3.98-3.66 (m, 4H), 3.26-3.07 (m, 3H), 2.98 (s, 1H), 2.72-2.41 (m, 2H), 2.33-2.02 (m, 3H), 1.98-1.73 (m, 3H), 1.56-1.43 (m, 3H). |
| 726a | 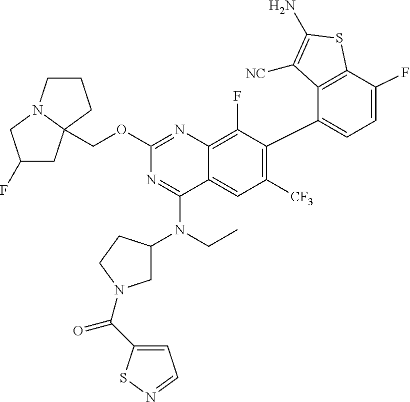 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2ccns2)C1] | |
| 727 | 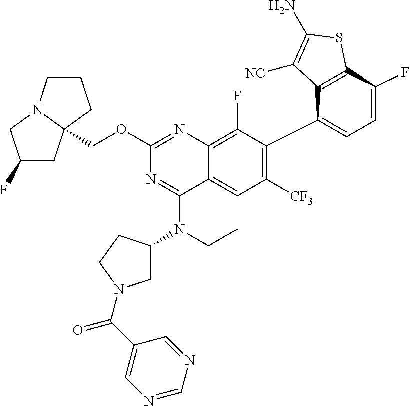 | LCMS m/z [M − 1]: 782.3 ¹H NMR (400 MHz, CD₃OD) δ = 9.26 (d, J = 14.0, 1H), 9.03 (d, J = 8.5, 2H), 8.26 (d, J = 22.8, 1H), 7.22 (s, 1H), 7.02 (d, J = 5.9, 1H), 5.43 (d, J = 12.6, 0.5H), 5.30 (d, J = 11.1, 0.5H), 5.10 (d, J = 29.1, 1H), 4.61-4.30 (m, 2H), 4.22 (d, J = 10.4, 1H), 4.04 (s, 1H), 3.89-3.72 (m, 4H), 3.64-3.40 (m, 3H), 3.13 (s, 1H), 2.57-1.89 (m, 8H), 1.46 (dd, J = 12.6, 6.5, 3H). |
| 727a | 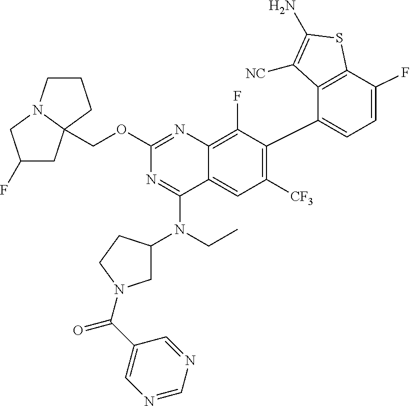 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2cncnc2)C1] | |
| 728 | 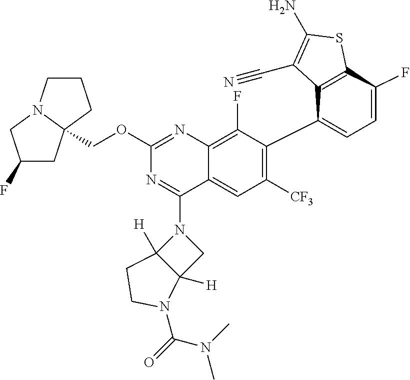 | LCMS m/z [M − 1]: 731.1 ¹H NMR (400 MHz, CD₃OD) δ = 8.10 (s, 1H), 7.22- 7.18 (m, 1H), 7.01 (t, J = 8.9, 1H), 5.56 (s, 1H), 5.44 (s, 0.5H), 5.30 (s, 0.5H), 4.90 (s, 2H), 4.38 (dd, J = 32.5, 11.2, 2H), 4.05 (d, J = 7.9, 1H), 3.63-3.42 (m, 5H), 3.13 (s, 1H), 2.93 (s, 6H), 2.51-1.96 (m, 8H). |
| 728a | 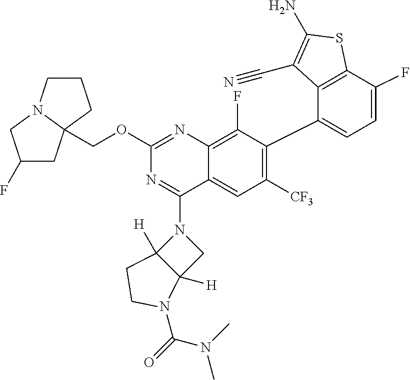 [SMILES: CN(C)C(=O)N1CCC2C1CN2c1nc(OCC23CCCN2CC(F)C3)nc2c(F)c(-c3ccc(F)c4sc(N)c(C#N)c34)c(C(F)(F)F)cc12] | |
| 729 | 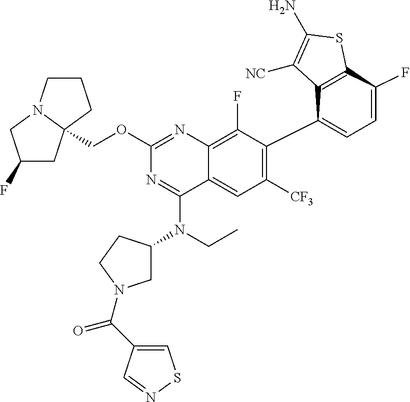 | LCMS m/z [M − 1]: 787.2 ¹H NMR (400 MHz, CD₃OD) δ 9.36 (d, 1H), 8.81 (s, 1H), 8.27 (d, 1H), 7.26-7.18 (m, 1H), 7.06-6.98 (m, 1H), 5.45 (d, 0.5H), 5.31 (d, 0.5H), 5.15-5.02 (m, 1H), 4.51-4.42 (m, 1H), 4.27-4.16 (m, 1H), 4.12-3.77 (m, 5H), 3.75-3.60 (m, 1H), 3.59-3.39 (m, 3H), 3.25-3.15 (m, 1H), 2.65-2.00 (m, 8H), 1.49 (t, 3H). |
| 729a | 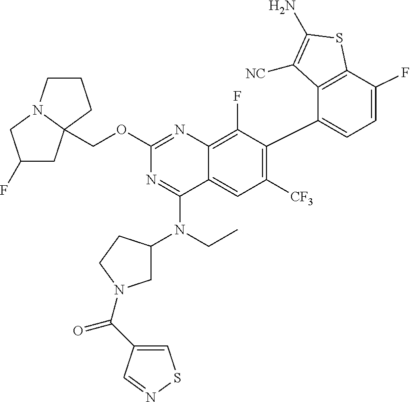 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2cnsc2)C1] | |
| 730 | 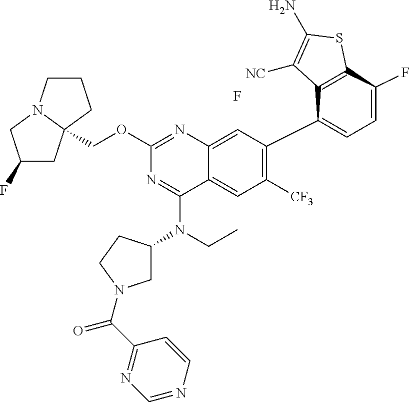 | LCMS m/z [M − 1]: 782.2 ¹H NMR (400 MHz, CD₃OD) δ 9.25 (d, J = 17.6 Hz, 1 H), 8.97 (d, J = 8.4 Hz, 1 H), 8.25 (d, J = 17.6 Hz, 1 H), 7.24-7.19 (m, 1H), 7.04-6.98 (m, 1H), 5.36-5.20 (m, 1H), 5.11-5.03 (m, 1H), 4.36-4.24 (m, 2H), 4.23-3.92 (m, 3H), 3.91-3.68 (m, 3H), 3.29-3.12 (m, 4H), 3.06- 2.95 (m, 1H), 2.56-1.83 (m, 8H), 1.51-1.42 (m, 3H). |
| 730a | 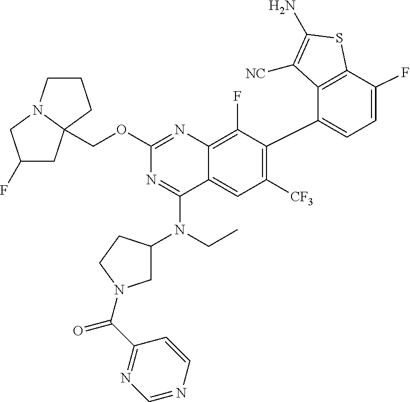 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2ccncn2)C1] | |
| 731 | 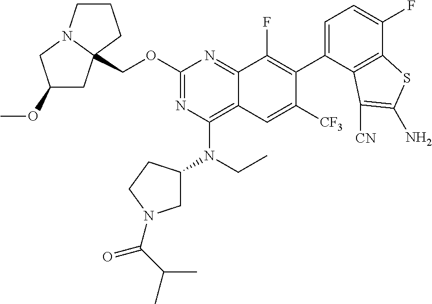 | LCMS m/z [M − 1]: 758.3 ¹H NMR (400 MHz, CD₃OD) δ 8.32 (s, 1H), 7.17-7.31 (m, 1H), 7.05 (t, J = 8.9 Hz, 1H), 5.05-5.19 (m, 1H), 4.75-4.85 (m, 2H), 4.63-4.74 (m, 1H), 3.81-4.30 (m, 7H), 3.54-3.76 (m, 2H), 3.37-3.53 (m, 2H), 3.20-3.23 (m, 2H), 2.79-2.97 (m, 1H), 2.41-2.64 (m, 4H), 2.21- 2.34 (m, 3H), 2.04-2.18 (m, 1H), 1.45-1.58 (m, 3H), 1.10-1.23 (m, 6H). |
| 731a | 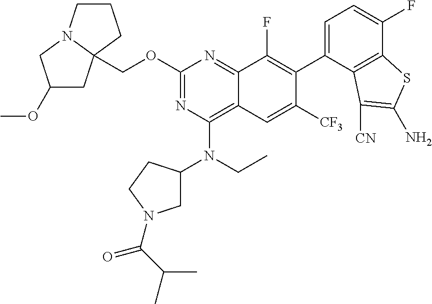 [SMILES: CCN(c1nc(OCC23CCCN2CC(OC)C3)nc2c(F)c(-c3ccc(F)c4sc(N)c(C#N)c34)c(C(F)(F)F)cc12)C1CCN(C(=O)C(C)C)C1] | |
| 732 | 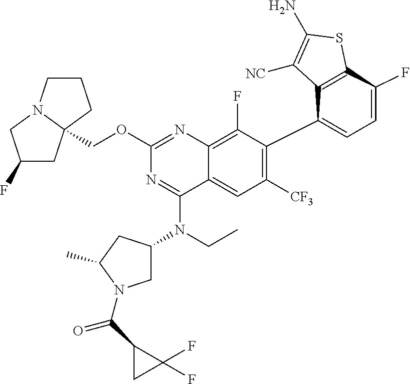 | LCMS m/z [M − 1]: 794.1 ¹H NMR (400 MHz, CD₃OD) δ 8.23-8.26 (m, 1H), 7.20-7.24 (m, 1H), 7.01 (t, 1H), 5.36 (s, 0.5H), 5.23 (m, 0.5H), 4.91-4.94 (m, 1H), 4.20-4.36 (m, 3H), 4.13-4.15 (m, 1H), 3.88-3.91 (m, 2H), 3.80 (t, 0.5H), 3.58 (t, 0.5H), 3.22-3.26 (m, 1H), 3.01-3.03 (m, 1H), 2.74-2.89 (m, 2H), 2.59-2.63 (m, 1H), 1.88-2.37 (m, 9H), 1.77- 1.78 (m, 1H), 1.40-1.54 (m, 6H). |
| 732a | 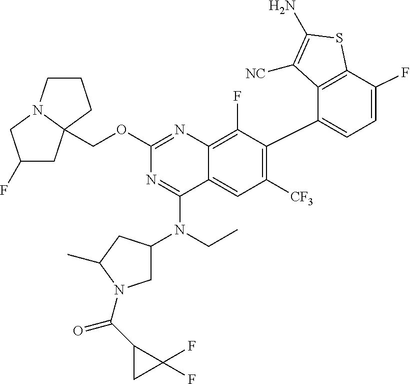 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)C2CC2(F)F)C1] | |
| 733 | 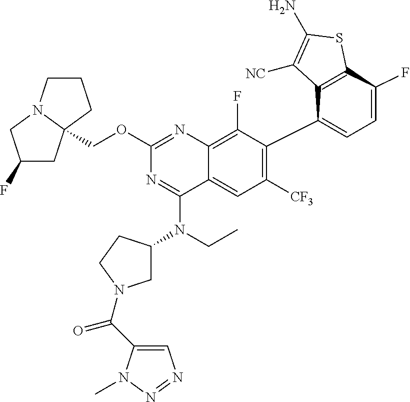 | LCMS m/z [M − 1]: 785.30 ¹H NMR (400 MHz, CD₃OD) δ 8.25 (d, 1H), 8.13 (d, 1H), 7.23-7.19 (m, 1H), 7.03-6.98 (m, 1H), 5.33 (d, 0.5H), 5.19 (d, 0.5H), 5.12-5.07 (m, 1H), 4.35-4.28 (m, 1H), 4.27-4.25 (m, 3H), 4.23-4.10 (m, 2H), 4.05-3.82 (m, 4H), 3.79-3.69 (m, 1H), 3.28-3.19 (m, 2H), 3.14- 2.95 (m, 2H), 2.57-2.44 (m, 2H), 2.26-2.05 (m, 3H), 1.99-1.80 (m, 3H), 1.46 (t, 3H). |
| 733a | 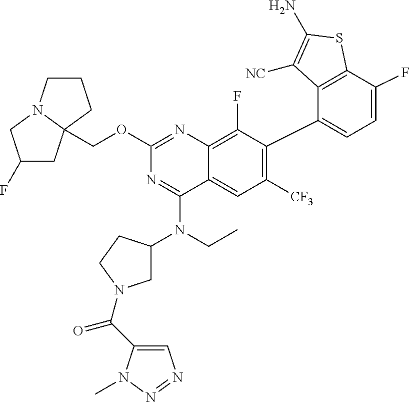 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2cnnn2C)C1] | |
| 734 | 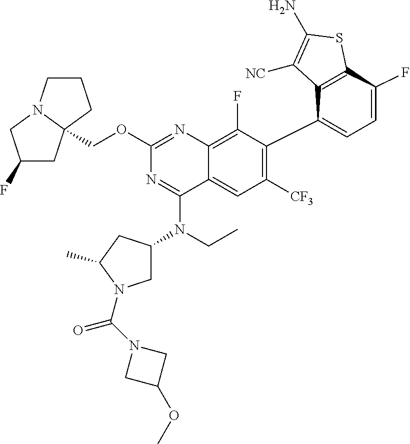 | LCMS m/z [M − 1]: 803.20 ¹H NMR (400 MHz, CD₃OD) δ 8.25 (s, 1H), 7.23-7.20 (m, 1H), 7.03-6.98 (m, 1H), 5.39 (s, 0.5H), 5.26 (s, 0.5H), 4.82-4.77 (m, 1H), 4.33-4.25 (m, 3H), 4.21- 4.17 (m, 1H), 4.04-3.98 (m, 2H), 3.95-3.83 (m, 4H), 3.69 (d, 1H), 3.44 (t, 1H), 3.28 (s, 3H), 3.26-3.19 (m, 2H), 3.07-3.00 (m, 1H), 2.55-2.48 (m, 1H), 2.41-2.14 (m, 3H), 2.12-2.08 (m, 1H), 2.05-1.94 (m, 3H), 1.93- 1.87 (m, 1H), 1.46 (t, 3H), 1.37 (d, 3H). |
| 734a | 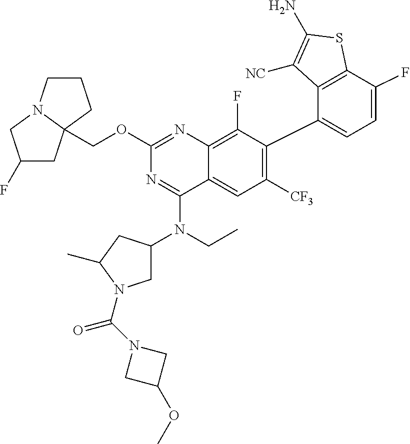 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)N2CC(OC)C2)C1] | |
| 735 | 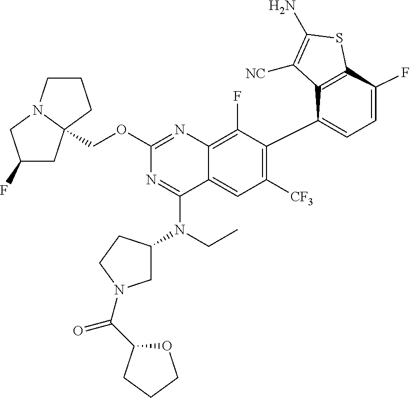 | LCMS m/z [M − 1]: 774.2 ¹H NMR (400 MHz, CD₃OD) δ 8.53 (brs, 0.65H), 8.32- 8.17 (m, 1H), 7.29-7.19 (m, 1H), 7.01 (t, J = 8.8 Hz, 1H), 5.44 (s, 0.53H), 5.31 (s, 0.61H), 5.03-4.94 (m, 1H), 4.67-4.57 (m, 1H), 4.47-4.23 (m, 2H), 4.08-3.64 (m, 7H), 3.64-3.34 (m, 4H), 3.20-3.08 (m, 1H), 2.61-1.84 (m, 12H), 1.56-1.37 (m, 3H). |
| 735a | 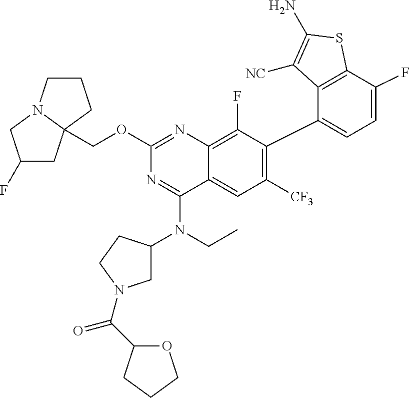 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CCCO2)C1] | |
| 736 | 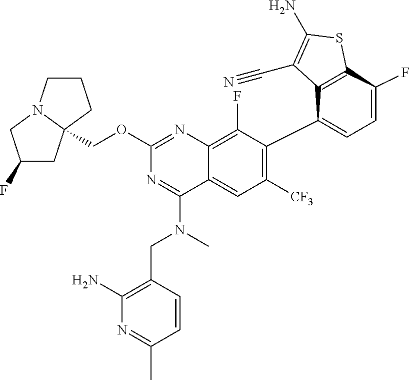 | LCMS m/z [M − 1]: 713.4 ¹H NMR (400 MHz, CD₃OD): δ 8.20 (s, 1 H), 7.45 (d, J = 7.6 Hz, 1 H), 7.18 (t, J = 2.8 Hz, 1 H), 6.99 (t, J = 9.2 Hz, 1 H), 6.60 (d, J = 7.6 Hz, 1 H), 5.28 (d, J = 54.8 Hz, 1 H), 4.86-4.85 (m, 2 H), 4.31-4.21 (m, 2 H), 3.50 (s, 3 H), 3.27-3.11 (m, 3 H), 3.05-2.97 (m, 1 H), 2.36 (s, 3 H), 2.32-2.07 (m, 3H), 2.04-1.93 (m, 2H), 1.92-1.86 (m, 1H). |
| 736a | 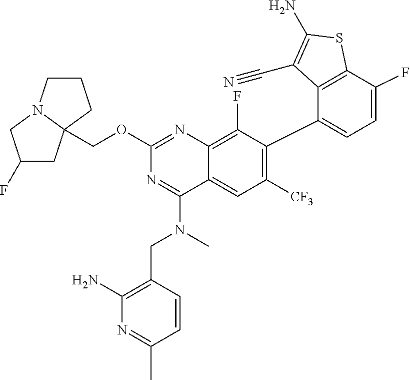 [SMILES: Cc1ccc(CN(C)c2nc(OCC34CCCN3CC(F)C4)nc3c(F)c(-c4ccc(F)c5sc(N)c(C#N)c45)c(C(F)(F)F)cc23)c(N)n1] | |
| 737 | 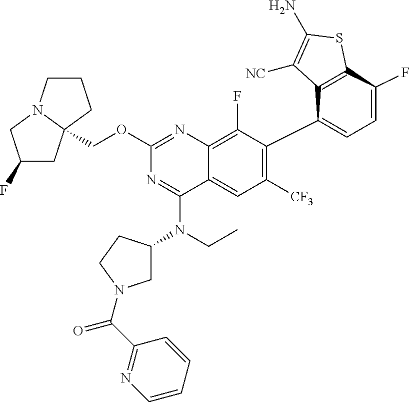 | LCMS m/z [M − 1]: 781.1 ¹H NMR (400 MHz, CD₃OD) δ 8.62 (dd, J = 4.4, 16.0 Hz, 1H), 8.24 (d, J = 22.8 Hz, 1H), 7.97-7.93 (m, 1H), 7.83- 7.80 (m, 1H), 7.54-7.48 (m, 1H), 7.22-7.19 (m, 1H), 7.03-6.99 (m, 1H), 5.34 (d, J = 21.2 Hz, 0.5H), 5.20 (d, J = 22 Hz, 0.5H), 5.13-5.02 (m, 1H), 4.34-4.15 (m, 3H), 4.07-3.70 (m, 5H), 3.21-3.16 (m, 3H), 3.12-2.98 (m, 1H), 2.58-1.82 (m, 8H), 1.50-1.45 (m, 3H). |
| 737a | 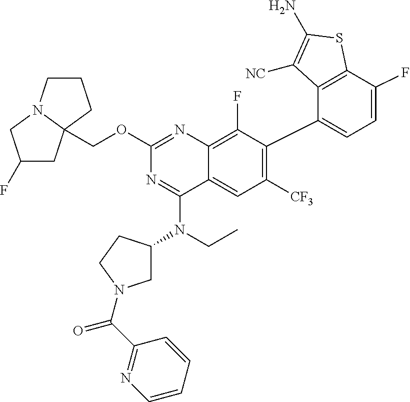 [SMILES: CCN(c1nc(OCC23CCCN2CC(F)C3)nc2c(F)c(-c3ccc(F)c4sc(N)c(C#N)c34)c(C(F)(F)F)cc12)[C@H]1CCN(C(=O)c2ccccn2)C1] | |
| 738 | 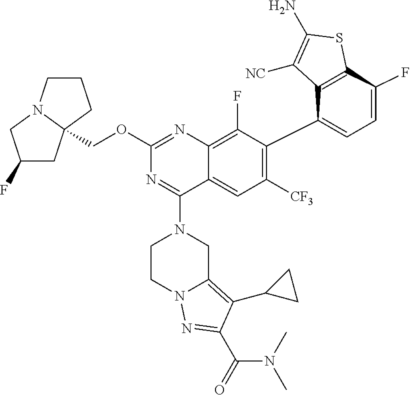 | LCMS m/z [M − 1]: 796.2 ¹H NMR (400 MHz, CD₃OD) δ 8.33 (s, 1H), 7.26- 7.23 (m, 1H), 7.06-7.01 (m, 1H), 5.58 (s, 0.53H), 5.45 (s, 0.54H), 5.28 (s, 2H), 4.70-4.67 (m, 1H), 4.59-4.41 (m, 5H), 3.90-3.70 (m, 3H), 3.38 (s, 1H), 3.21 (s, 3H), 3.11 (s, 3H), 2.86-2.09 (m, 6H), 1.67-1.61 (m, 1H), 0.82-0.80 (m, 2H), 0.54-0.41 (m, 2H). |
| 738a | 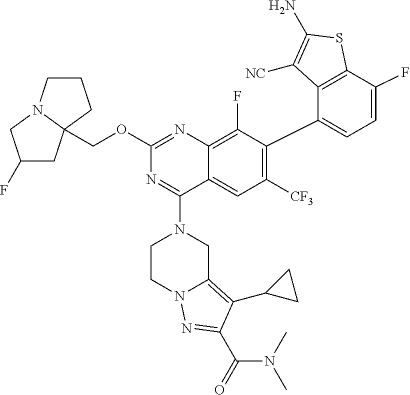 [SMILES: CN(C)C(=O)c1nn2c(c1C1CC1)CN(c1nc(OCC34CCCN3CC(F)C4)nc3c(F)c(-c4ccc(F)c5sc(N)c(C#N)c45)c(C(F)(F)F)cc13)CC2] | |
| 739 | 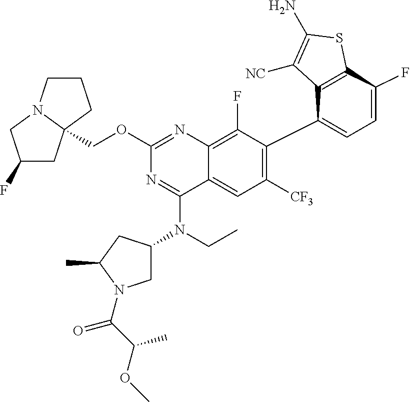 | LCMS m/z [M − 1]: 776.8 ¹H NMR (400 MHz, CD₃OD) δ 8.45 (s, 1H), 8.27 (s, 1H), 7.24-7.21 (m, 1H), 7.05-7.00 (m, 1H), 5.52 (s, 0.5H), 5.38 (s, 0.5H), 5.28-5.23 (m, 1H), 4.80-4.53 (m, 3H), 4.45-4.13 (m, 2H), 3.92-3.57 (m, 6H), 3.57 (s, 1H), 3.31 (s, 3H), 2.63-2.05 (m, 8H), 1.42-1.37 (m, 3H), 1.37-1.31 (m, 6H). |
| 739a | 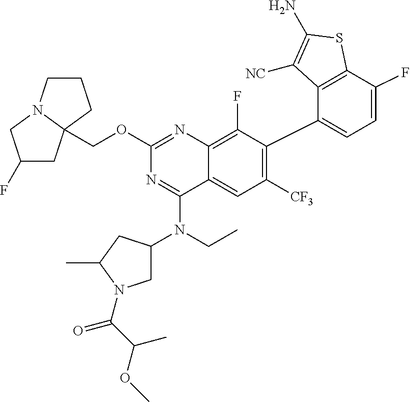 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)C(C)OC)C1] | |
| 740 | 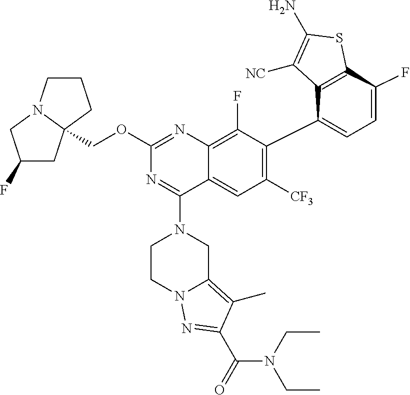 | LCMS m/z [M − 1]: 798.27 ¹H NMR (400 MHz, CD₃OD) δ 8.24 (s, 1H), 7.26-7.21 (m, 1H), 7.02 (t, J = 8.8 Hz, 1H), 5.39 (s, 0.5H), 5.25 (s, 0.5H), 5.12 (m, 2H), 4.52-4.27 (m, 6H), 3.61-3.44 (m, 4H), 3.29-3.20 (m, 3H), 3.09-2.99 (m, 1H), 2.39-2.23 (m, 2H), 2.21-2.14 (m, 1H), 2.08-1.85 (m, 6H), 1.24 (t, J = 6.9 Hz, 3H), 1.16 (t, J = 6.9 Hz, 3H). |
| 740a | 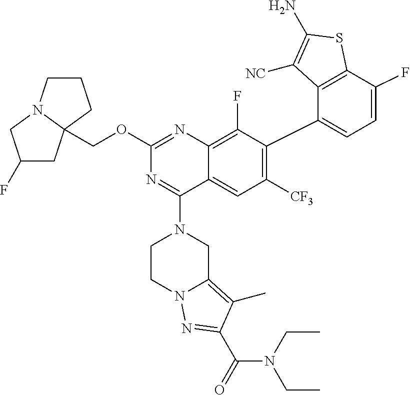 [SMILES: CCN(CC)C(=O)c1nn2c(c1C)CN(c1nc(OCC34CCCN3CC(F)C4)nc3c(F)c(-c4ccc(F)c5sc(N)c(C#N)c45)c(C(F)(F)F)cc13)CC2] | |
| 741 | 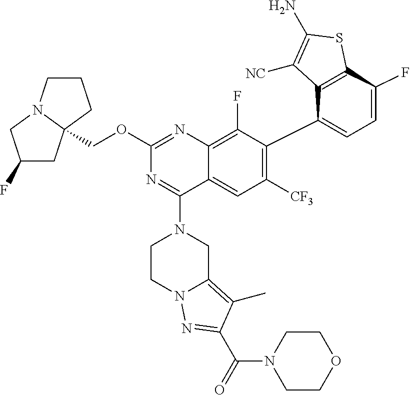 | LCMS m/z [M − 1]: 812.25 ¹H NMR (400 MHz, CD₃OD) δ 8.24 (s, 1H), 7.26-7.21 (m, 1H), 7.03 (t, J = 8.9 Hz, 1H), 5.40 (s, 0.5H), 5.27 (s, 0.5H), 5.12 (m, 2H), 4.52-4.28 (m, 7H), 3.78-3.63 (m, 8H), 3.28-3.23 (m, 1H), 3.15-3.01 (m, 2H), 2.41-2.25 (m, 2H), 2.22-2.15 (m, 1H), 2.11-2.07 (m, 3H), 2.06- 1.98 (m, 2H), 1.97-1.89 (m, 1H). |
| 741a | 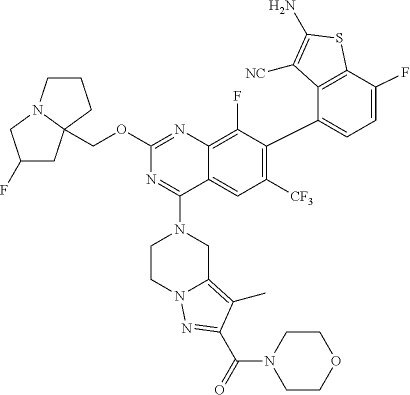 [SMILES: Cc1c(C(=O)N2CCOCC2)nn2c1CN(c1nc(OCC34CCCN3CC(F)C4)nc3c(F)c(-c4ccc(F)c5sc(N)c(C#N)c45)c(C(F)(F)F)cc13)CC2] | |
| 742 | 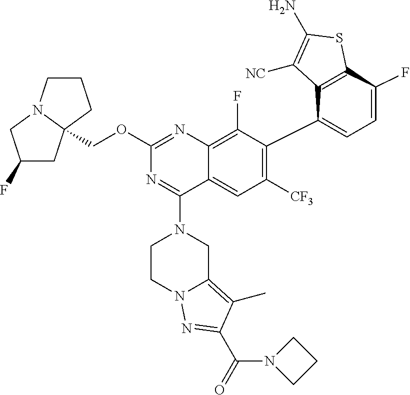 | LCMS m/z [M − 1]: 782.24 ¹H NMR (400 MHz, CD₃OD) δ 8.23 (s, 1H), 7.26-7.19 (m, 1H), 7.05-6.97 (m, 1H), 5.36 (s, 0.5H), 5.23 (s, 0.5H), 5.28 (s, 2H), 4.59-4.51 (m, 2H), 4.49-4.40 (m, 3H), 4.38-4.24 (m, 3H), 4.17-4.10 (m, 2H), 3.28-3.16 (m, 3H), 3.06-2.95 (m, 1H), 2.40-2.26 (m, 3H), 2.25- 2.12 (m, 5H), 2.05-1.84 (m, 3H). |
| 742a | 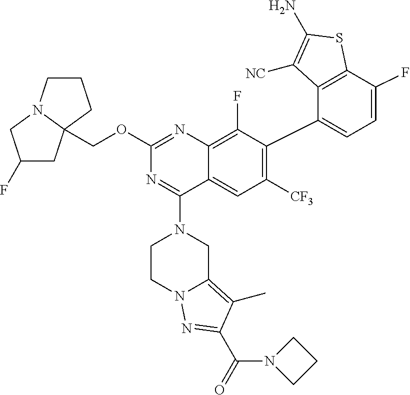 [SMILES: Cc1c(C(=O)N2CCC2)nn2c1CN(c1nc(OCC34CCCN3CC(F)C4)nc3c(F)c(-c4ccc(F)c5sc(N)c(C#N)c45)c(C(F)(F)F)cc13)CC2] | |
| 743 | 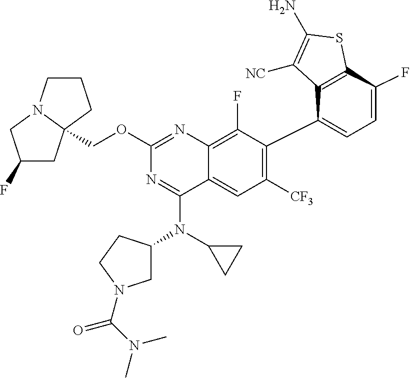 | LCMS m/z [M − 1]: 759.2 ¹H NMR (400 MHz, CD₃OD) δ = 8.66 (s, 1H), 7.21 (d, J = 4.8, 1H), 7.00 (t, J = 8.8, 1H), 5.35 (s, 0.5H), 5.21 (s, 0.5H), 4.80-4.70 (m, 1H), 4.22 (dd, J = 27.4, 10.3, 2H), 4.04 (d, J = 7.3, 1H), 3.79-3.39 (m, 5H), 3.18 (d, J = 18.3, 2H), 2.98 (s, 1H), 2.88 (s, 6H), 2.65-2.46 (m, 2H), 2.29-1.85 (m, 6H), 1.01 (s, 2H), 0.71 (d, J = 12.9, 2H). |
| 743a | 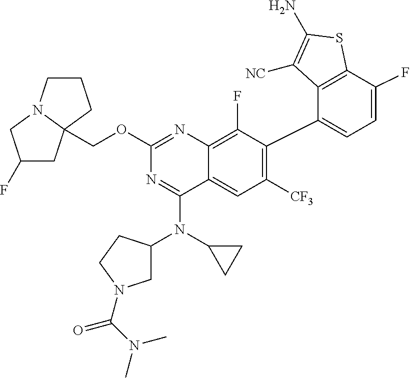 [SMILES: CN(C)C(=O)N1CCC(N(c2nc(OCC34CCCN3CC(F)C4)nc3c(F)c(-c4ccc(F)c5sc(N)c(C#N)c45)c(C(F)(F)F)cc23)C2CC2)C1] | |
| 744 | 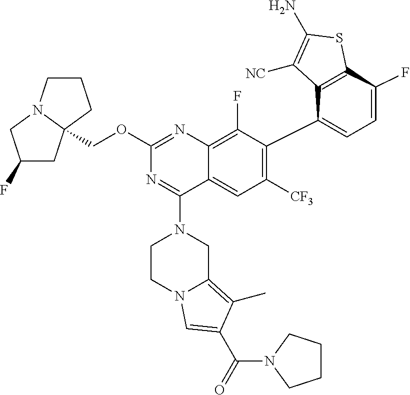 | LCMS m/z [M − 1]: 796.25 ¹H NMR (400 MHz, CD₃OD) δ 8.29-8.26 (m, 1H), 7.27-7.20 (m, 1H), 7.06-6.99 (m, 1H), 5.61 (s, 0.5H), 5.48 (s, 0.5H), 5.15 (s, 2H), 4.77-4.63 (m, 2H), 4.53- 4.38 (m, 4H), 4.06-3.67 (m, 5H), 3.63-3.52 (m, 2H), 3.48-3.37 (m, 1H), 2.77-2.50 (m, 2H), 2.48-2.38 (m, 1H), 2.36-2.24 (m, 2H), 2.22-2.06 (m, 4H), 1.98-1.86 (m, 4H). |
| 744a | 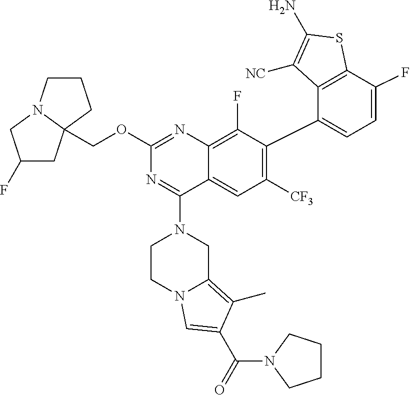 [SMILES: Cc1c(C(=O)N2CCCC2)cn2c1CN(c1nc(OCC34CCCN3CC(F)C4)nc3c(F)c(-c4ccc(F)c5sc(N)c(C#N)c45)c(C(F)(F)F)cc13)CC2] | |
| 745 | 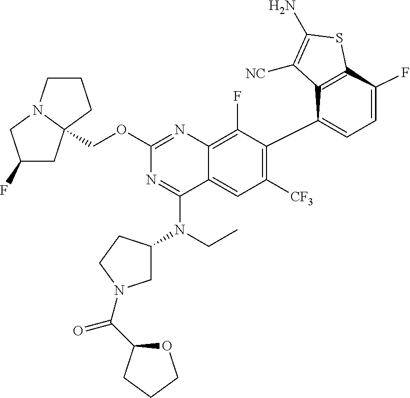 | LCMS m/z [M − 1]: 774.3 ¹H NMR (400 MHz, CD₃OD) δ = 8.26 (d, J = 7.9, 1H), 7.22 (dd, J = 8.2, 5.1, 1H), 7.02 (t, J = 8.9, 1H), 5.47 (d, J = 3.7, 0.5H), 5.34 (d, J = 3.1, 0.5H), 5.11-4.93 (m, 1H), 4.74-4.57 (m, 1H), 4.40 (dt, J = 17.3, 11.7, 2H), 4.31- 4.02 (m, 1H), 4.02-3.43 (m, 10H), 3.24-3.12 (m, 1H), 2.65-1.92 (m, 12H), 1.48 (t, J = 6.4 Hz, 3H). |
| 746 | 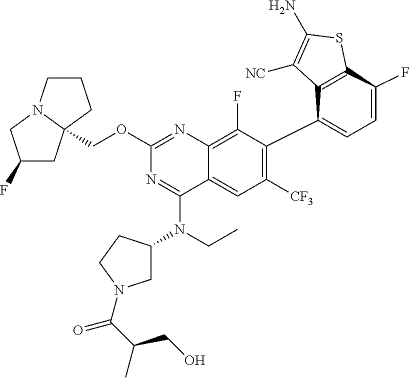 | LCMS m/z [M − 1]: 762.3 ¹H NMR (400 MHz, CD₃OD) δ = 8.28 (d, J = 9.5 Hz, 1H), 7.22 (dd, J = 8.1, 5.0, 1H), 7.02 (t, J = 8.9 Hz, 1H), 5.49 (s, 0.5H), 5.36 (s, 0.5H), 5.16-5.00 (m, 1H), 4.45 (dt, J = 24.7, 11.7, 2H), 4.35-4.05 (m, 1H), 4.00-3.83 (m, 3H), 3.76-3.47 (m, 7H), 3.26-3.18 (m, 1H), 3.02- 2.89 (m, 1H), 2.35 (dddd, J = 109.4, 58.4, 35.3, 21.6, 8H), 1.48 (dt, J = 13.3, 6.9, 3H), 1.07 (t, J = 6.0, 3H). |
| 746a | 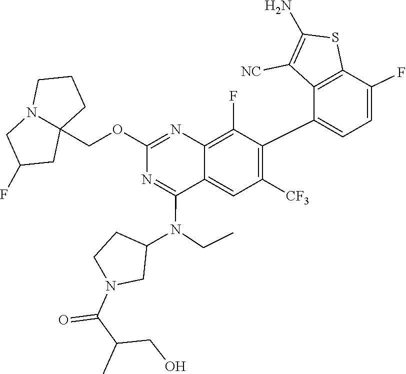 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C(C)CO)C1] | |
| 747 | 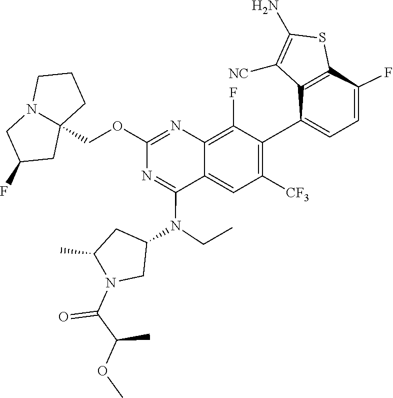 | LCMS m/z [M − 1]: 776.3 ¹H NMR (400 MHz, CD₃OD) δ 8.24 (s, 1H), 7.24-7.18 (m, 1H), 7.04-6.98 (t, 1H), 5.37-5.33 (m, 0.5H), 5.24- 5.20 (m, 0.5H), 4.84-4.77 (m, 1H), 4.35-4.31 (d, 1H), 4.26-4.13 (m, 3H), 4.12-4.02 (m, 1H), 3.93-3.83 (m, 2H), 3.78-3.69 (m, 3H), 3.30 (s, 3H), 3.25-3.18 (m, 2H), 3.04-2.97 (m, 1H), 2.65-2.55 (m, 1H), 2.65-2.55 (m, 1H), 2.33-1.82 (m, 8H), 1.48-1.38 (m, 3H), 1.34 (t, 3H). |
| 748 | 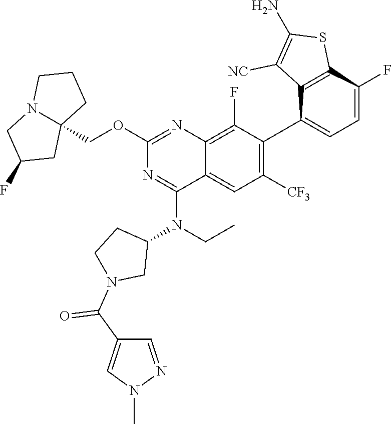 | LCMS m/z [M − 1]: 784.3 ¹H NMR (400 MHz, CD₃OD) δ 8.45 (s, 1H), 8.29 (s, 1H), 8.13 (d, 1H), 7.90 (d, 1H), 7.25-7.19 (m, 1H), 7.02 (t, 1H), 5.43 (d, 0.5H), 5.34 (d, 0.5H), 5.11-5.04 (m, 1H), 4.59-4.41 (m, 2H), 4.38-4.27 (m, 1H), 4.16-4.09 (m, 1H), 4.05-4.73 (m, 7H), 3.68-3.52 (m, 3H), 3.26- 3.19 (m, 1H), 2.66-2.49 (m, 2H), 2.45-2.32 (m, 2H), 2.22-2.12 (m, 2H), 2.01-1.89 (m, 2H), 1.59-1.43 (m, 3H). |
| 748a | 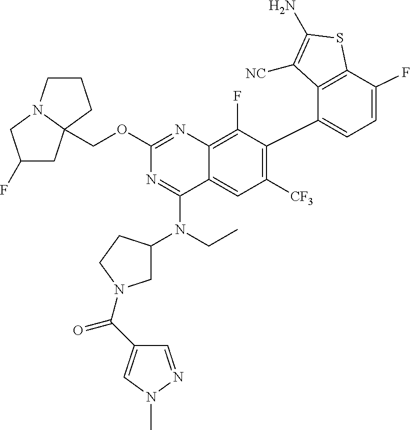 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2cnn(C)c2)C1] | |
| 749 | 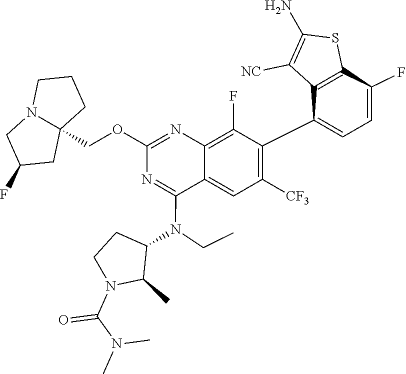 | LCMS m/z [M − 1]: 761.2 ¹H NMR (400 MHz, CD₃OD) δ 8.25 (s, 1H), 7.21-7.25 (m, 1H), 7.01 (t, 1H), 5.37 (s, 0.5H), 5.23 (s, 0.5H), 4.61-4.68 (m, 1H), 4.39-4.44 (m, 1H), 4.30-4.32 (m, 1H), 4.19-4.21 (m, 1H), 3.90-3.97 (m, 1H), 3.74-3.81 (m, 1H), 3.58-3.63 (m, 1H), 3.47-3.52 (m, 1H), 3.12- 3.24 (m, 3H), 2.98-3.05 (m, 1H), 2.88 (s, 6H), 2.44-2.51 (m, 1H), 2.09-2.38 (m, 4H), 1.85-2.01 (m, 3H), 1.44 (t, 3H), 1.21 (d, 3H). |
| 749a | 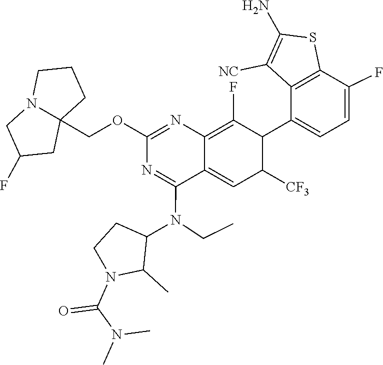 [SMILES: CCN(c1nc(OCC23CCCN2CC(F)C3)nc2c1=CC(C(F)(F)F)C(c1ccc(F)c3sc(N)c(C#N)c13)C=2F)C1CCN(C(=O)N(C)C)C1C] | |
| 750 | 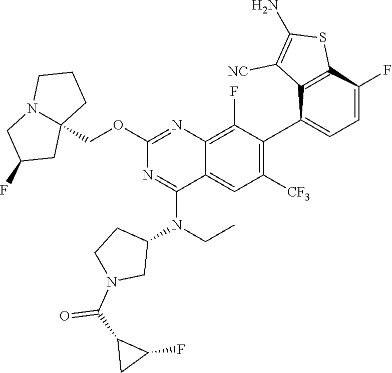 | LCMS m/z [M − 1]: 762.2 ¹H NMR (400 MHz, CD₃OD) δ 8.254 (s, 1H), 7.253- 7.179 (m, 1H), 7.015 (t, 1H), 5.402 (s, 0.5H), 5.265 (s, 0.5H), 5.114 (t, 0.5H), 5.0.8-4.998 (m, 0.5H), 4.848- 4.755 (m, 1H), 4.429-4.012 (m, 4H), 4.004-3.793 (m, 3H), 3.778-3.772 (m, 0.5H), 3.682-3.626 (m, 0.5H), 3.517-3.404 (m, 1H), 3.388-3.316 (m, 2H), 3.121-3.039 (m, 1H), 2.624-2.416 (m, 2H), 2.334-2.117 (m, 3H), 2.113-1.843 (m, 4H), 1.755-1.655 (m, 1H), 1.557-1.406 (m, 3H), 1.144-1.064 (m, 1H). |
| 751 | 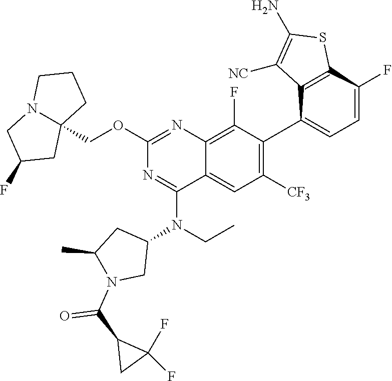 | LCMS m/z [M − 1]: 794.1 ¹H NMR (400 MHz, CD₃OD) δ 8.26 (d, J = 8.4 Hz, 1 H), 7.23 (t, J = 7.6 Hz, 1 H), 7.02 (t, J = 9.2 Hz, 1 H), 5.44-5.27 (m, 2H), 4.52-4.29 (m, 3H), 4.17 (t, J = 10.4 Hz, 1 H), 3.97-3.78 (m, 3H), 3.51-3.38 (m, 2H), 3.13 (s, 1H), 2.93-2.79 (m, 1H), 2.77-2.64 (m, 1H), 2.49-1.97 (m, 8H), 1.91-1.66 (m, 2H), 1.48 (t, J = 6.8 Hz, 3 H), 1.29 (t, J = 3.2 Hz, 1 H). |
| 752 | 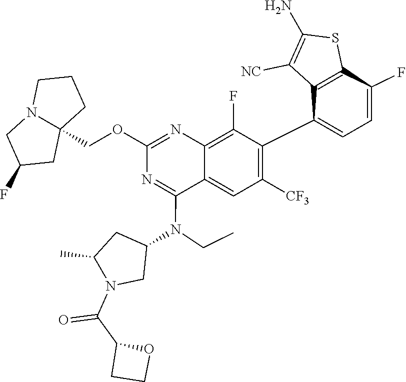 | LCMS m/z [M − 1]: 774.2 ¹H NMR (400 MHz, CD₃OD) δ 8.22 (s, 1H), 7.23-7.19 (m, 1H), 7.01 (t, 1H), 5.39 (s, 0.5H), 5.32 (t, 1H), 5.25 (s, 0.5H), 4.82-4.80 (m, 1H), 4.71-4.66 (m, 1H), 4.59- 4.54 (m, 1H), 4.39-4.37 (m, 1H), 4.25-4.23 (m, 1H), 4.19-4.13 (m, 1H), 3.91-3.85 (m, 3H), 3.69-3.64 (m, 1H), 3.13-2.94 (m, 3H), 2.87-2.81 (m, 1H), 2.61-2.55 (m, 1H), 2.33-2.11 (m, 4H), 2.09-1.97 (m, 4H), 1.94- 1.89 (m, 1H), 1.47 (d, 3H), 1.42 (t, 3H). |
| 752a | 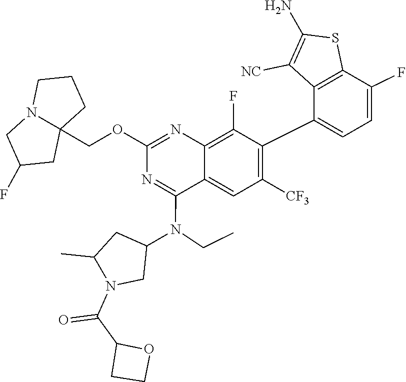 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)C2CCO2)C1] | |
| 753 | 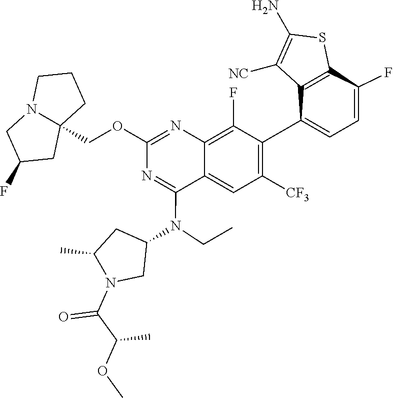 | LCMS m/z [M − 1]: 776.2 ¹H NMR (400 MHz, CD₃OD) δ 8.24 (s, 1H), 7.23-7.20 (m, 1H), 7.01 (t, 1H), 5.36 (s, 0.5H), 5.22 (s, 0.5H), 4.89-4.87 (s, 1H), 4.37-4.30 (m, 2H), 4.26-4.18 (m, 2H), 4.15-4.09 (m, 1H), 3.90-3.85 (m, 2H), 3.69-3.63 (m, 1H), 3.34 (s, 3H), 3.26-3.19 (m, 2H), 3.05-2.99 (m, 1H), 2.61-2.54 (m, 1H), 2.31-2.07 (m, 4H), 2.05-1.95 (m, 3H), 1.93-1.87 (m, 1H), 1.44-1.38 (m, 6H), 1.35 (d, 3H). |
| 754 | 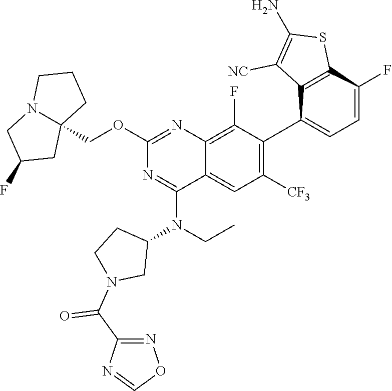 | LCMS m/z [M − 1]: 772.1 ¹H NMR (400 MHz, CD₃OD) δ 9.44 (d, 1H), 8.24 (s, 1H), 7.26-7.12 (m, 1H), 7.06-6.92 (m, 1H), 5.40-5.31 (m, 0.5H), 5.26-5.18 (m, 0.5H), 5.08-4.97 (m, 1H), 4.43-4.11 (m, 3H), 4.04-3.74 (m, 3H), 3.73-3.32 (m, 3H), 3.22-3.12 (m, 2H), 3.07-2.96 (m, 1H), 2.61-2.06 (m, 5H), 2.04-1.77 (m, 3H), 1.61-1.36 (m, 3H). |
| 754a | 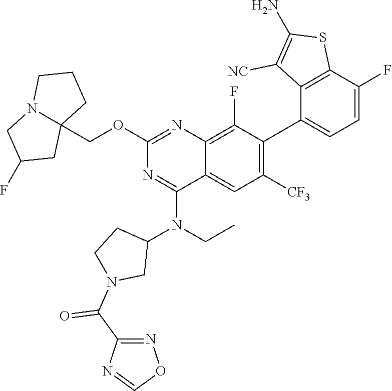 | |
| 755 | 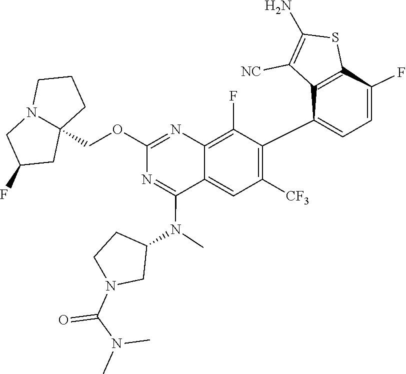 | LCMS m/z [M − 1]: 733.1 ¹H NMR (400 MHz, CD₃OD) δ 8.30 (s, 1H), 7.23-7.19 (m, 1H), 7.00 (t, 1H), 5.40-5.36 (m, 0.5H), 5.26-5.24 (m, 0.5H), 5.24-5.17 (m, 1H), 4.34-4.25 (dq, 2H), 3.87- 3.80 (m, 1H), 3.66-3.52 (m, 3H), 3.45 (s, 3H), 3.35-3.31 (m, 1H), 3.28-3.22 (m, 2H), 3.07-3.00 (m, 1H), 2.90 (s, 6H), 2.36-2.13 (m, 5H), 2.04-1.96 (m, 2H), 1.94-1.86 (m, 1H). |
| 755a | 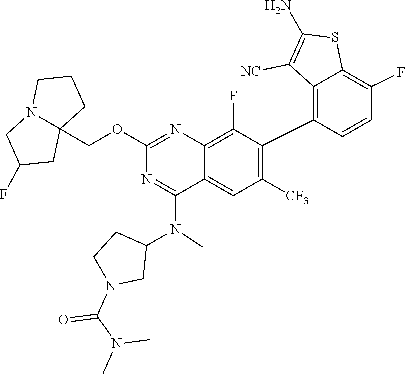 [SMILES: CN(C)C(=O)N1CCC(N(C)c2nc(OCC34CCCN3CC(F)C4)nc3c(F)c(-c4ccc(F)c5sc(N)c(C#N)c45)c(C(F)(F)F)cc23)C1] | |
| 756 | 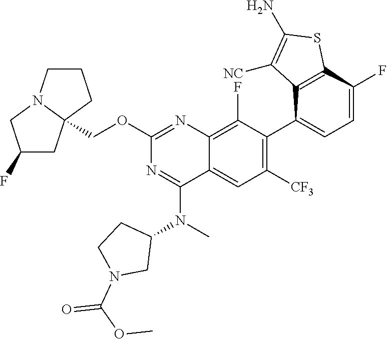 | LCMS m/z [M − 1]: 720.1 ¹H NMR (400 MHz, CD₃OD) δ 8.29 (s, 1H), 7.24-7.19 (m, 1H), 7.00 (t, 1H), 5.40-5.35 (m, 0.5H), 5.32-5.24 (m, 1.5H), 4.34-4.25 (dq, 2H), 3.97-3.90 (m, 1H), 3.76- 3.67 (m, 1H), 3.72 (s, 3H), 3.54-3.42 (m, 2H), 3.44 (s, 3H), 3.35-3.31 (m, 1H), 3.28-3.22 (m, 2H), 3.07-3.00 (m, 1H), 2.40-2.27 (m, 3H), 2.25-2.14 (m, 2H), 2.04- 1.88 (m, 3H). |
| 756a | 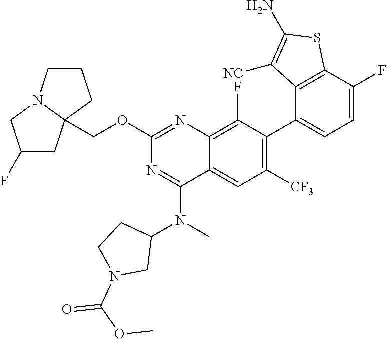 [SMILES: COC(=O)N1CCC(N(C)c2nc(OCC34CCCN3CC(F)C4)nc3c(F)c(-c4ccc(F)c5sc(N)c(C#N)c45)c(C(F)(F)F)cc23)C1] | |
| 757 | 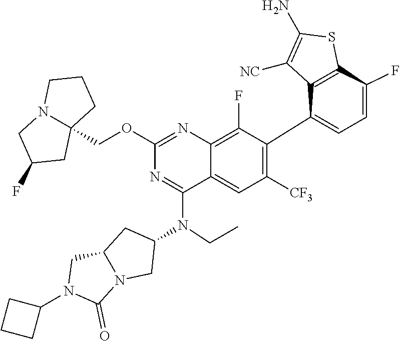 | LCMS m/z [M − 1]: 785.2 ¹H NMR (400 MHz, CD₃OD) δ 8.20 (s, 1H), 7.26-7.15 (m, 1H), 7.07-6.93 (m, 1H), 5.40-5.19 (m, 2H), 4.55- 4.17 (m, 3H), 3.98-3.84 (m, 1H), 3.81-3.49 (m, 6H), 3.41-3.31 (m, 2H), 3.28-3.25 (m, 1H), 3.12-2.97 (m, 1H), 2.40-1.89 (m, 12H), 1.77-1.66 (m, 2H), 1.39- 1.33 (m, 3H). |
| 757a | 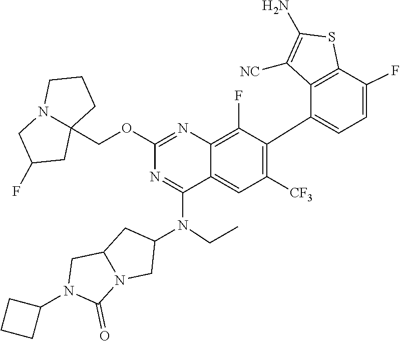 [SMILES: CCN(c1nc(OCC23CCCN2CC(F)C3)nc2c(F)c(-c3ccc(F)c4sc(N)c(C#N)c34)c(C(F)(F)F)cc12)C1CC2CN(C3CCC3)C(=O)N2C1] | |
| 758 | 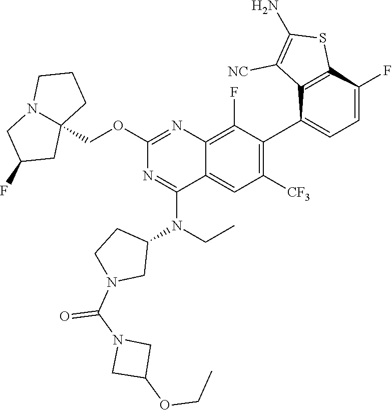 | LCMS m/z [M − 1]: 803.28. ¹H NMR (400 MHz, CD₃OD) δ 8.27 (s, 1H), 7.26-7.20 (m, 1H), 7.03 (t, J = 8.7 Hz, 1H), 5.53 (s, 0.5H), 5.40 (s, 0.5H), 5.04-4.94 (m, 1H), 4.56-4.41 (m, 2H), 4.32-4.16 (m, 3H), 3.99-3.80 (m, 5H), 3.78-3.69 (m, 2H), 3.67- 3.56 (m, 3H), 3.52-3.42 (m, 3H), 3.26-3.20 (m, 1H), 2.59-2.41 (m, 3H), 2.39-2.26 (m, 2H), 2.24-2.14 (m, 2H), 2.10-2.00 (m, 1H), 1.49 (t, J = 6.8 Hz, 3H), 1.20 (t, J = 6.4 Hz, 3H). |
| 758a | 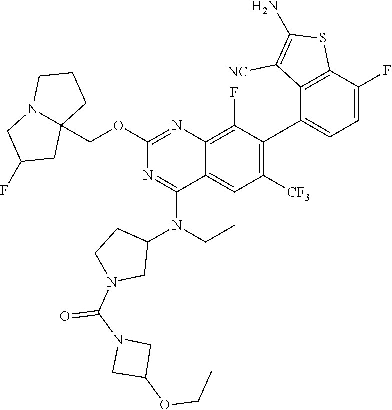 [SMILES: CCOC1CN(C(=O)N2CCC(N(CC)c3nc(OCC45CCCN4CC(F)C5)nc4c(F)c(-c5ccc(F)c6sc(N)c(C#N)c56)c(C(F)(F)F)cc34)C2)C1] | |
| 759 | 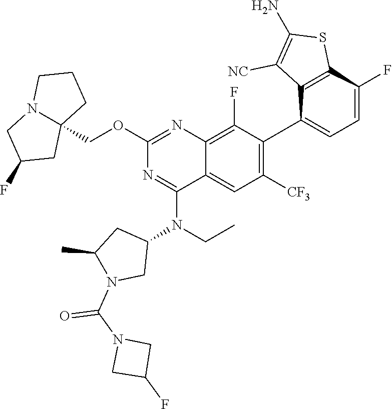 | LCMS m/z [M − 1]: 791.2. ¹H NMR (400 MHz, CD₃OD) δ 8.22 (s, 1H), 7.23-7.20 (m, 1H), 7.01 (t, J = 8.8 Hz, 1H), 5.37-5.17 (m, 2H), 4.40-4.00 (m, 7H), 3.91-3.85 (m, 2H), 3.79-3.74 (m, 1H), 3.63-3.58 (m, 1H), 3.27-3.20 (m, 3H), 3.02-3.00 (m, 1H), 2.64-2.61 (m, 1H), 2.34-1.88 (m, 8H), 1.43 (t, J = 7.2 Hz, 3H), 1.26 (d, J = 8.4 Hz, 3H). |
| 759a | 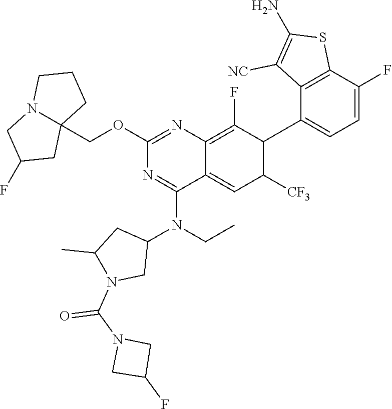 | |
| 760 | 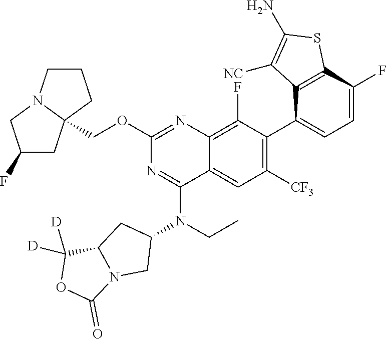 | LCMS m/z [M − 1]: 734.22 ¹H NMR (400 MHz, CD₃OD) δ 8.18 (s, 1H), 7.24-7.18 (m, 1H), 7.06-6.97 (m, 1H), 5.42 (s, 0.5H), 5.29 (s, 0.5H), 5.01-4.90 (m, 1H), 4.41-4.24 (m, 2H), 4.23-4.16 (m, 1H), 3.94-3.69 (m, 3H), 3.67-3.31 (m, 4H), 3.16- 3.06 (m, 1H), 2.55-2.17 (m, 5H), 2.13-1.86 (m, 3H), 1.56-1.42 (m, 3H). |
| 760a | 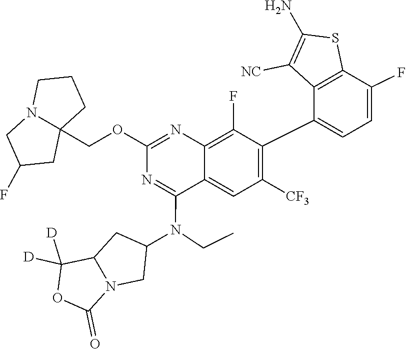 [SMILES: [2H]C1([2H])OC(=O)N2CC(N(CC)c3nc(OCC45CCCN4CC(F)C5)nc4c(F)c(-c5ccc(F)c6sc(N)c(C#N)c56)c(C(F)(F)F)cc34)CC21] | |
| 761 | 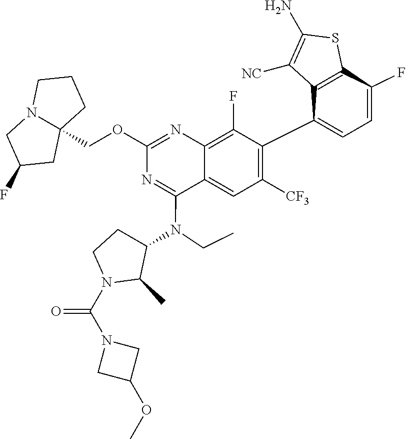 | LCMS m/z [M − 1]: 803.2 ¹H NMR (400 MHz, CD₃OD) δ 8.21 (s, 1H), 7.20-7.24 (m, 1H), 7.01 (t, 1H), 5.39 (s, 0.5H), 5.25 (s, 0.5H), 4.42-4.59 (m, 2H), 4.19-4.32 (m, 4H), 4.04-4.07 (m, 1H), 3.89-3.97 (m, 2H), 3.71-3.80 (m, 2H), 3.62-3.67 (m, 1H), 3.37-3.50 (m, 2H), 3.30 (s, 3H), 3.12-3.23 (m, 2H), 3.01-3.07 (m, 1H), 2.55-2.65 (m, 1H), 2.20-2.40 (m, 3H), 2.10-2.16 (m, 1H), 1.89-2.03 (m, 3H), 1.46 (t, 3H), 1.31 (d, 3H). |
| 761a | 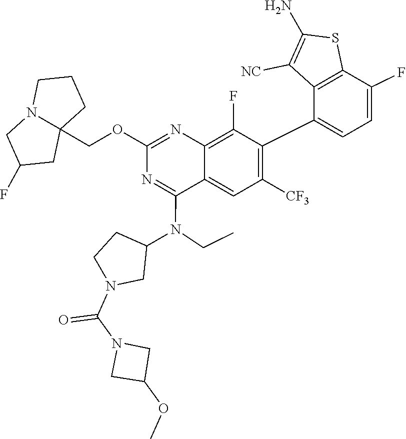 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2CC(OC)C2)C1] | |
| 762 | 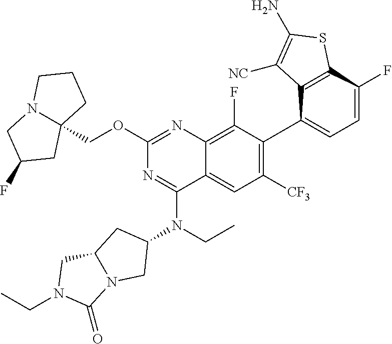 | LCMS m/z [M − 1]: 759.26 ¹H NMR (400 MHz, CD₃OD) δ 8.20 (s, 1H), 7.24-7.18 (m, 1H), 7.04-6.97 (m, 1H), 5.38 (s, 0.5H), 5.29-5.18 (m, 1.5H), 4.37-4.21 (m, 2H), 3.95-3.84 (m, 1H), 3.82- 3.44 (m, 6H), 3.39-3.31 (m, 2H), 3.29-3.19 (m, 3H), 3.08-2.98 (m, 1H), 2.40-2.11 (m, 4H), 2.05-1.84 (m, 4H), 1.44-1.32 (m, 3H), 1.20-1.10 (m, 3H). |
| 762a | 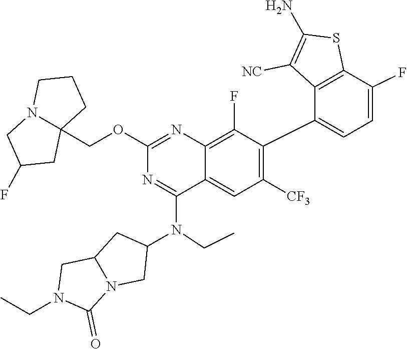 [SMILES: CCN1CC2CC(N(CC)c3nc(OCC45CCCN4CC(F)C5)nc4c(F)c(-c5ccc(F)c6sc(N)c(C#N)c56)c(C(F)(F)F)cc34)CN2C1=O] | |
| 763 | 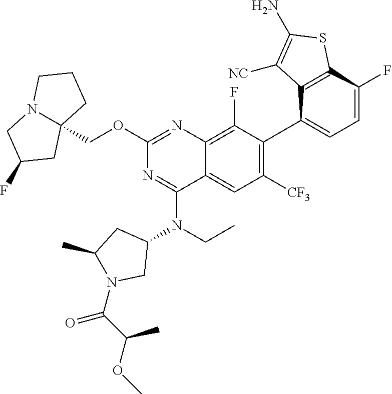 | LCMS m/z [M − 1]: 776.3 ¹H NMR (400 MHz, CD₃OD) δ 8.50 (brs, 0.79H), 8.24 (s, 1H), 7.28-7.16 (m, 1H), 7.01 (t, J = 8.8 Hz, 1H), 5.44 (s, 0.54H), 5.31 (s, 0.59H), 5.27-5.13 (m, 1H), 4.54- 4.03 (m, 5H), 3.99-3.72 (m, 3H), 3.54-3.34 (m, 3H), 3.31 (s, 3H), 3.19-3.31 (m, 1H), 2.66-1.94 (m, 8H), 1.49-1.15 (m, 9H). |
| 764 | 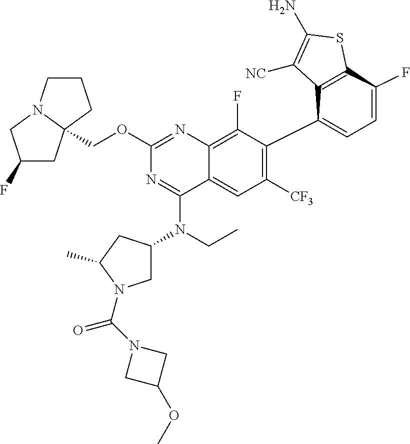 | LCMS m/z [M − 1]: 803.2 ¹H NMR (400 MHz, CD₃OD) δ 8.22 (s, 1H), 7.25-7.18 (m, 1H), 7.01 (t, J = 9.0 Hz, 1H), 5.39 (s, 0.56H), 5.26 (s, 0.54H), 5.22-5.10 (m, 1H), 4.50-4.05 (m, 6H), 0.41- 3.67 (m, 5H), 3.64-3.55 (m, 1H), 3.49-3.47 (m, 1H), 3.29 (s, 3H), 3.25-2.99 (m, 2H), 2.68-2.58 (m, 1H), 2.42-1.82 (m, 8H), 1.43 (t, J = 7.0 Hz, 3H), 1.26 (t, J = 7.2 Hz, 3H). |
| 764a | 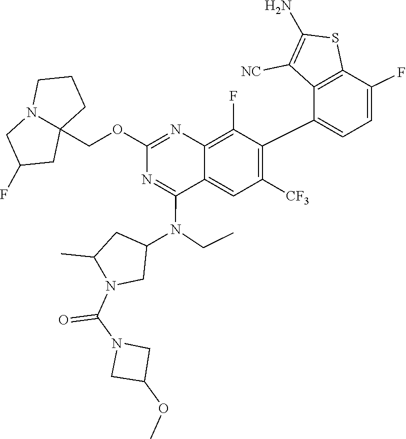 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)N2CC(OC)C2)C1] | |
| 765 | 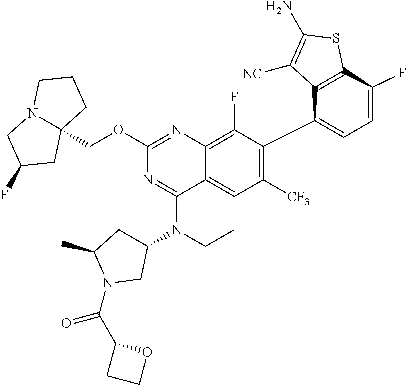 | LCMS m/z [M − 1]: 764.78 ¹H NMR (400 MHz, CD₃OD) δ 8.18-8.24 (m, 1H), 7.19-7.25 (m, 1H), 6.98-7.05 (m, 1H), 5.40 (s, 0.5H), 5.26 (s, 0.5H), 4.92-5.13 (m, 2H), 4.55-4.84 (m, 3H), 4.23-4.39 (m, 2H), 4.06-4.19 (m, 2H), 3.88-4.00 (m, 1H), 3.77-3.85 (m, 1H), 3.61-3.70 (m, 1H), 3.34-3.49 (m, 2H), 3.01-3.12 (m, 1H), 2.75-2.87 (m, 1H), 2.12- 2.50 (m, 5H), 1.88-2.08 (m, 3H), 1.06-1.19 (m, 6H). |
| 766 | 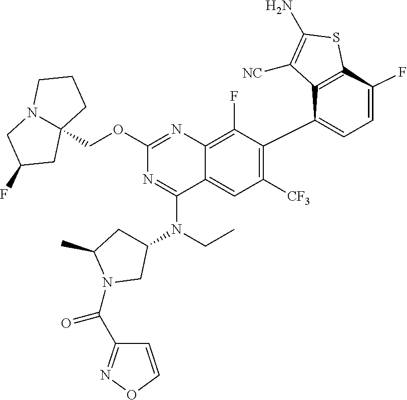 | LCMS m/z [M − 1]: 785.78 ¹H NMR (400 MHz, CD₃OD) δ 8.84-8.79 (m, 1H), 8.28-8.26 (m, 1H), 7.25-7.2 (m, 1H) 7.02-6.99 (m, 1H), 6.83-6.81 (m, 1H), 5.52 (s, 0.5H), 5.38 (s, 0.5H), 5.30- 5.23 (m, 1H), 5.00-4.87 (m, 1H), 4.70-4.45 (m, 3H), 4.40-3.59 (m, 6H), 3.31-3.13 (m, 1H), 2.81-2.70 (m, 1H), 2.42-1.99 (m, 7H), 1.57-1.17 (m, 6H). |
| 766a | 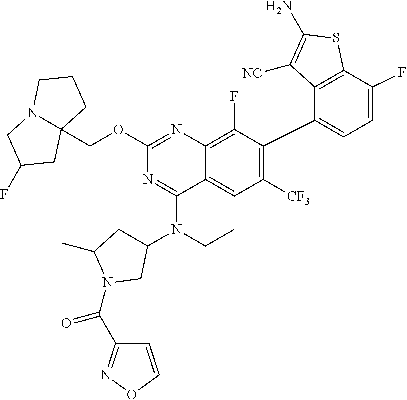 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)c2ccon2)C1] | |
| 767 | 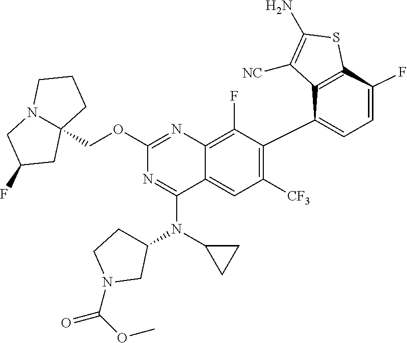 | LCMS m/z [M − 1]: 746.3 ¹H NMR (400 MHz, CD₃OD) δ = 8.69 (s, 1H), 7.22 (dd, J = 8.1, 5.1, 1H), 7.01 (t, J = 8.9, 1H), 5.43 (s, 0.5H), 5.30 (s, 0.5H), 4.82 (s, 1H), 4.33 (d, J = 7.9, 2H), 4.11 (t, J = 8.9, 1H), 3.82-3.56 (m, 5H), 3.55-3.34 (m, 5H), 3.12 (s, 1H), 2.71-2.00 (m, 8H), 1.03 (d, J = 6.2, 2H), 0.72 (s, 2H). |
| 767a | 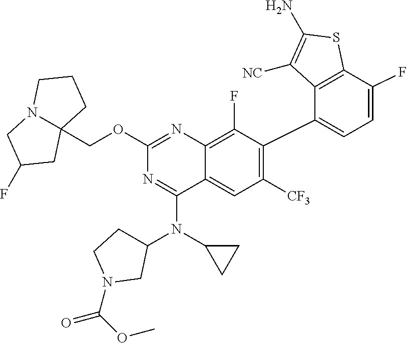 [SMILES: COC(=O)N1CCC(N(c2nc(OCC34CCCN3CC(F)C4)nc3c(F)c(-c4ccc(F)c5sc(N)c(C#N)c45)c(C(F)(F)F)cc23)C2CC2)C1] | |
| 768 | 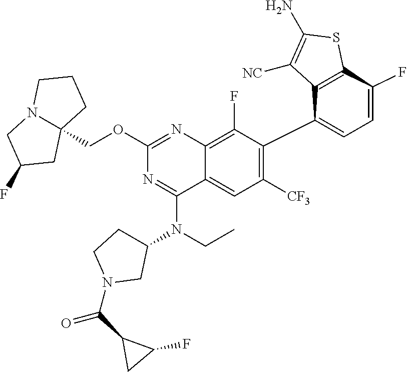 | LCMS m/z [M − 1]: 762.2 ¹H NMR (400 MHz, CD₃OD) δ 8.25 (d, 1H), 7.24-7.18 (m, 1H), 7.01 (t, 1H), 5.37 (s, 0.5H), 5.23 (s, 0.5H), 5.13-5.07 (m, 0.5H), 5.02-4.97 (m, 0.5H), 4.90-4.87 (m, 0.5H), 4.73-4.67 (m, 0.5H), 4.32-4.22 (m, 2H), 4.13- 4.07 (m, 0.5H), 4.01-3.75 (m, 4H), 3.63-3.58 (m, 0.5H), 3.48-3.34 (m, 1H), 3.29-3.20 (m, 3H), 3.06-2.98 (m, 1H), 2.64-2.40 (m, 2H), 2.36-2.10 (m, 4H), 2.03-1.86 (m, 3H), 1.51-1.43 (m, 3H), 1.34-1.25 (m, 2H) |
| 769 | 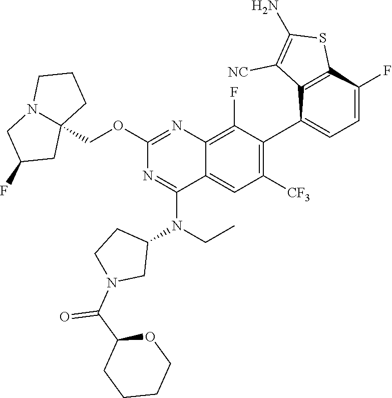 | LCMS m/z [M − 1]: 788.2 ¹H NMR (400 MHz, CD₃OD) δ = 8.24 (s, 1H), 7.22 (dd, J = 8.3, 5.1 Hz, 1H), 7.01 (t, J = 8.9, 1H), 5.40 (s, 0.5H), 5.27 (s, 0.5H), 5.06-4.94 (m, 1H), 4.38-3.68 (m, 9H), 3.66- 3.34 (m, 5H), 3.27-3.06 (m, 1H), 2.66-2.18 (m, 5H), 2.03-1.58 (m, 9H), 1.47 (dt, J = 16.7, 6.9, 3H). |
| 769a | 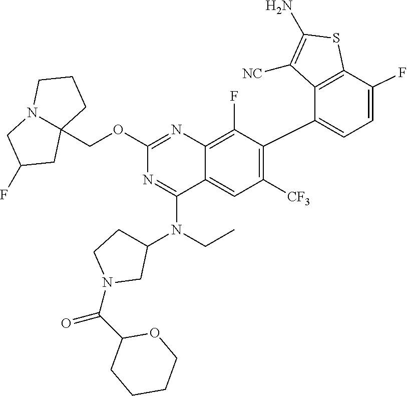 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CCCCO2)C1] | |
| 770 | 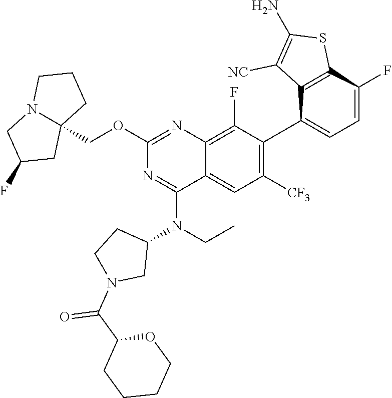 | LCMS m/z [M − 1]: 7.88.2 ¹H NMR (400 MHz, CD₃OD) δ = 8.24 (s, 1H), 7.22 (dd, J = 8.3, 5.1, 1H), 7.01 (t, J = 8.9, 1H), 5.39 (s, 0.5H), 5.25 (s, 0.5H), 5.06-4.94 (m, 1H), 4.38-3.68 (m, 9H), 3.66- 3.34 (m, 3H), 3.27-2.98 (m, 3H), 2.54-2.12 (m, 5H), 2.08-1.54 (m, 9H), 1.47 (dt, J = 16.7, 6.9, 3H). |
| 771 | 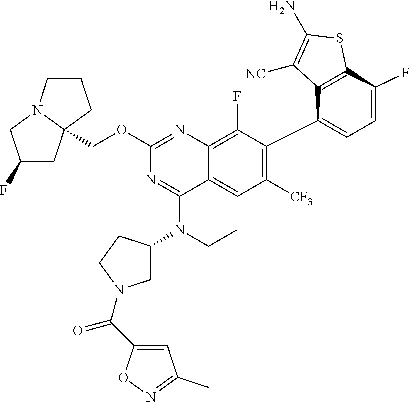 | LCMS m/z [M − 1]: 785.2 ¹H NMR (400 MHz, CD₃OD) δ 8.28 (s, 1H), 7.22 (d, J = 6.8 Hz, 1H), 7.00 (s, 1H), 6.88 (d, J = 5.6 Hz, 1H), 5.45 (s, 0.56H), 5.31 (s, 0.62H), 5.06 (d, J = 21.2 Hz, 1H), 4.43-3.84 (m, 8H), 3.69 (s, 1H), 3.48 (s, 2H), 3.18 (s, 1H), 2.63-1.94 (m, 11H), 1.53-1.49 (m, 3H). |
| 771a | 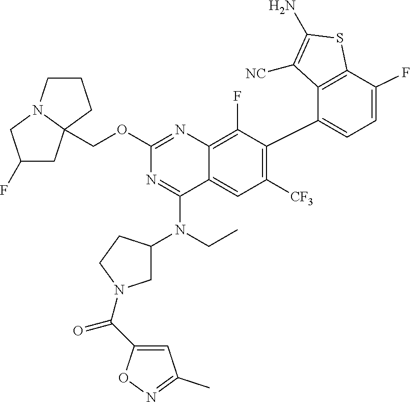 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2cc(C)no2)C1] | |
| 772 | 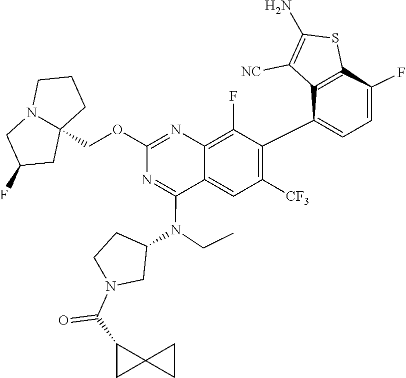 | LCMS m/z [M − 1]: 770.3 ¹H NMR (400 MHz, CD₃OD) δ 8.23 (s, 1H), 7.28-7.16 (m, 1H), 7.01 (t, 1H), 5.35 (s, 0.5H), 5.23 (s, 0.5H), 5.10-5.00 (m, 1H), 4.30-4.17 (m, 2H), 4.08-3.97 (m, 1H), 3.90-3.81 (m, 3H), 3.76-3.40 (m, 2H), 3.23-3.12 (m, 3H), 3.00 (s, 1H), 2.54-2.17 (m, 5H), 2.15-1.94 (m, 3H), 1.90-1.82 (m, 1H), 1.57-1.51 (m, 1H), 1.50-1.40 (m, 3H), 0.99-0.80 (m, 5H). |
| 772a | 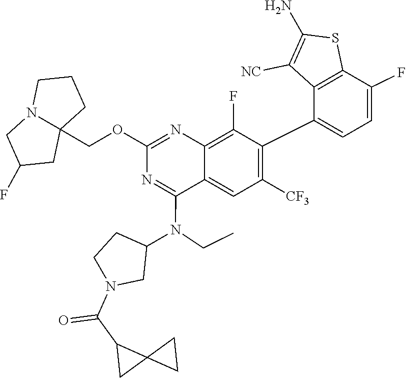 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CC23CC3)C1] | |
| 773 | 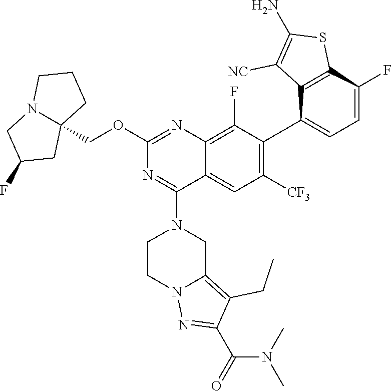 | LCMS m/z [M − 1]: 784.2 ¹H NMR (400 MHz, CD₃OD) δ 8.24 (s, 1H), 7.25-7.22 (m, 1H), 7.04-7.00 (m, 1H), 5.38 (s, 0.53H), 5.24 (s, 0.54H), 5.15 (s, 2H), 4.49-4.28 (m, 6H), 3.28-3.20 (m, 3H), 3.10-2.93 (m, 7H), 2.55-2.51 (m, 2H), 2.35-2.14 (m, 3H), 2.03-1.89 (m, 3H), 1.17-1.13 (m, 3H). |
| 773a | 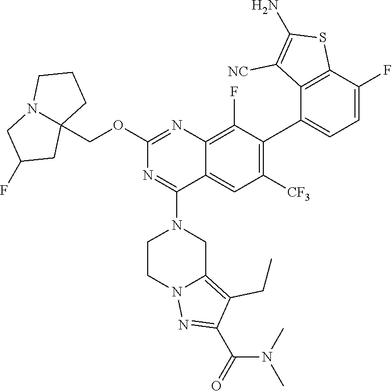 | |
| 774 | 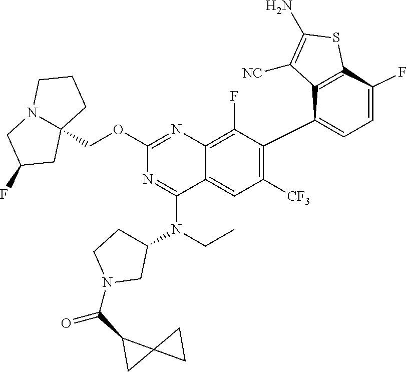 | LCMS m/z [M − 1]: 770.3 ¹H NMR (400 MHz, CD₃OD) δ 8.24 (d, 1H), 7.21 (t, 1H), 7.01 (t, 1H), 5.36 (s, 0.5H), 5.22 (s, 0.5H), 5.16- 4.98 (m, 1H), 4.29-4.22 (m, 2H), 4.07-3.98 (m, 1H), 3.88-3.82 (m, 2H), 3.71-3.36 (m, 3H), 3.23-3.12 (m, 3H), 2.99 (s, 1H), 2.62-2.18 (m, 5H), 2.15-2.08 (m, 1H), 2.05-1.96 (s, 2H), 1.90-1.80 (m, 1H), 1.53-1.43 (m, 4H), 0.96-0.85 (m, 5H). |
| 775 | 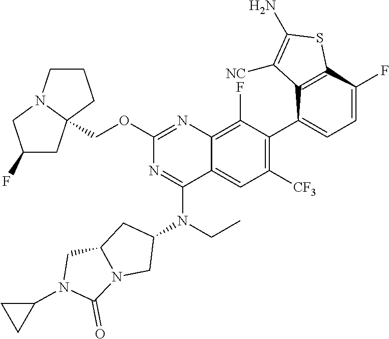 | LCMS m/z [M − 1]: 771.2 ¹H NMR (400 MHz, CD₃OD) δ 8.20 (s, 1H), 7.23-7.19 (m, 1H), 7.03-6.99 (m, 1H), 5.42 (s, 0.5H), 5.29 (s, 0.5H), 5.23-5.18 (m, 1H), 4.42-4.31 (m, 2H), 3.86-3.37 (m, 10H), 3.12 (s, 1H), 2.58-2.54 (m, 1H), 2.43-2.20 (m, 4H), 2.07-1.93 (m, 4H), 1.40-1.28 (m, 3H), 0.79- 0.65 (m, 4H). |
| 775a | 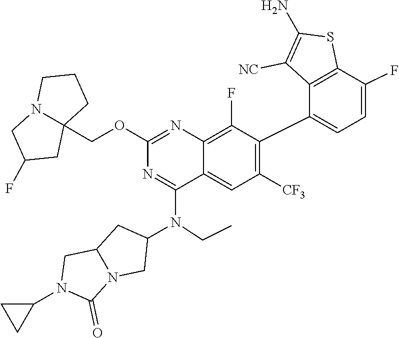 [SMILES: CCN(c1nc(OCC23CCCN2CC(F)C3)nc2c(F)c(-c3ccc(F)c4sc(N)c(C#N)c34)c(C(F)(F)F)cc12)C1CC2CN(C3CC3)C(=O)N2C1] | |
| 776 | 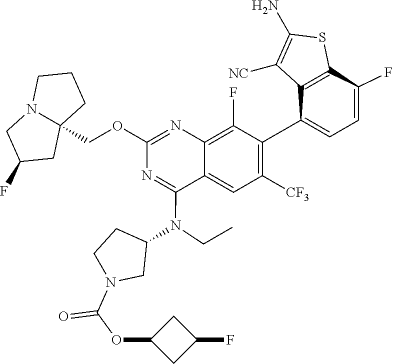 | LCMS m/z [M − 1]: 792.3 ¹H NMR (400 MHz, CD₃OD) δ 8.23 (s, 1H), 7.24-7.19 (m, 1H), 7.01 (t, 1H), 5.73 (s, 0.5H), 5.24 (s, 0.5H), 5.50-4.97 (m, 1H), 4.83-4.78 (m, 0.5H), 4.69 (s, 0.5H), 4.61-4.51 (m, 1H), 4.33-4.23 (m, 2H), 4.00-3.90 (m, 1H), 3.89-3.82 (m, 2H), 3.79-3.70 (m, 1H), 3.61-3.53 (m, 1H), 3.50-3.32 (m, 2H), 3.27-3.21 (m, 2H), 3.06- 2.99 (m, 1H), 2.94-2.84 (m, 2H), 2.51-2.42 (m, 1H), 2.40-2.21 (m, 5H), 2.16-2.10 (m, 1H), 2.04-1.96 (m, 2H), 1.93-1.85 (m, 1H), 1.45 (t, 3H). |
| 776a | 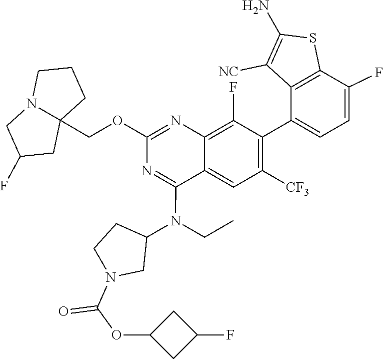 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2CC(F)C2)C1] | |
| 777 | 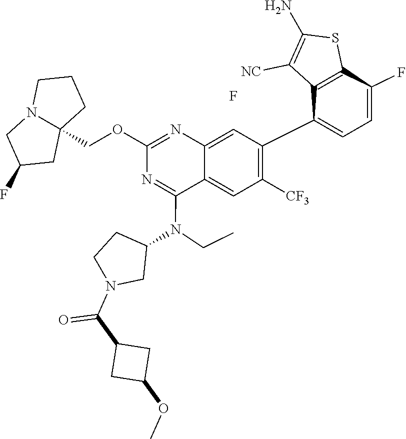 | LCMS m/z [M − 1]: 788.2 ¹H NMR (400 MHz, CD₃OD) δ 8.23 (d, 1H), 7.20-7.23 (m, 1H), 7.01 (t, 1H), 5.37 (s, 0.5H), 5.23 (s, 0.5H), 4.99-5.04 (m, 1H), 4.19-4.30 (m, 2H), 3.97-4.01 (m, 1H), 3.84-3.87 (m, 4H), 3.41-3.64 (m, 3H), 3.22-3.24 (m, 5H), 3.02-3.05 (m, 1H), 2.85-2.91 (m, 1H), 1.88- 2.51 (m, 12H), 1.42-1.47 (m, 3H). |
| 777a | 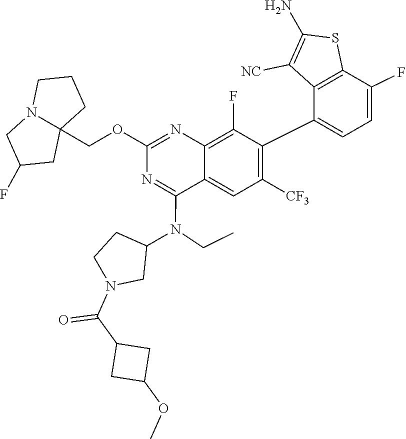 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CC(OC)C2)C1] | |
| 778 | 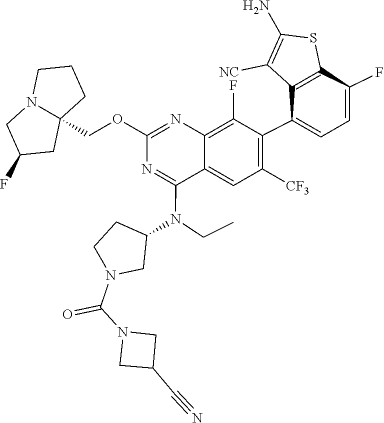 | LCMS m/z [M − 1]: 784.2 ¹H NMR (400 MHz, CD₃OD) δ 8.23 (s, 1H), 7.19-7.23 (m, 1H), 7.01 (t, 1H), 5.38 (s, 0.5H), 5.24 (s, 0.5H), 4.94-5.00 (m, 1H), 4.36 (t, 1H), 4.22-4.30 (m, 3H), 4.14 (m, 1H), 3.79-3.89 (m, 3H), 3.54-3.69 (m, 3H), 3.41- 3.48 (m, 1H), 3.33-3.37 (m, 1H), 3.24-3.28 (m, 2H), 3.00-3.03 (m, 1H), 2.11-2.48 (m, 6H), 1.96-2.02 (m, 2H), 1.90 (t, 1H), 1.45 (m, 3H). |
| 778a | 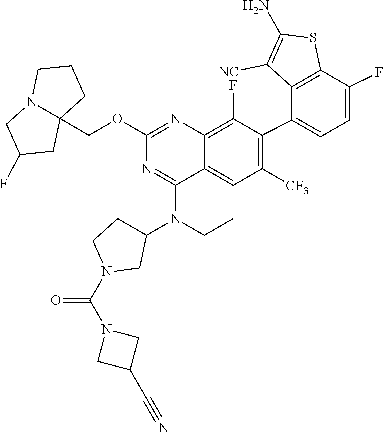 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2CC(C#N)C2)C1] | |
| 779 | 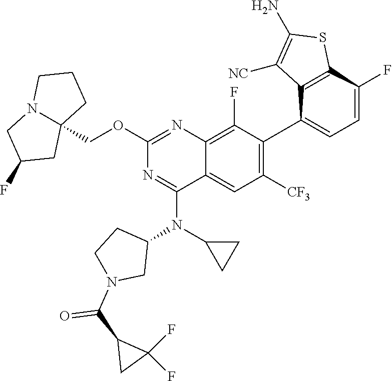 | LCMS m/z [M − 1]: 792.77 ¹H NMR (400 MHz, CD₃OD) δ 8.74-8.71 (m, 1H), 7.25- 7.22 (m, 1H), 7.04-6.99 (m, 1H), 5.48 (s, 0.5H), 5.35 (s, 0.5H), 4.93-4.86 (m, 1H), 4.35-4.18 (m, 3H), 3.83-3.47 (m, 7H), 3.25-3.13 (m, 1H), 2.85-2.44 (m, 3H), 2.44- 1.80 (m, 8H), 1.17-1.03 (m, 1H), 0.76-0.71 (m, 1H). |
| 779a | 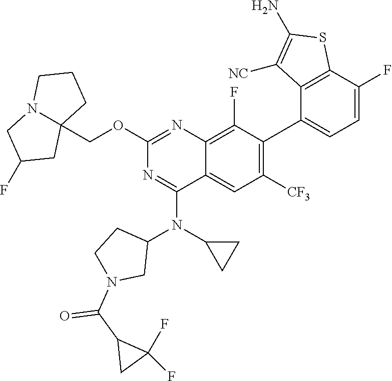 [SMILES: N#Cc1c(N)sc2c(F)ccc(-c3c(C(F)(F)F)cc4c(N(C5CC5)C5CCN(C(=O)C6CC6(F)F)C5)nc(OCC56CCCN5CC(F)C6)nc4c3F)c12] | |
| 780 | 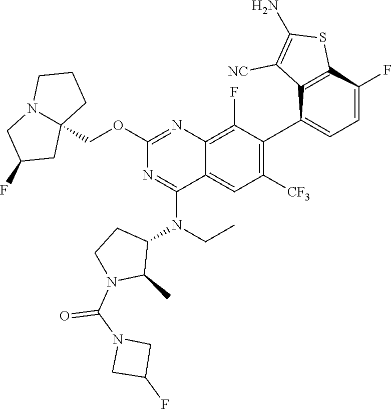 | LCMS m/z [M − 1]: 791.2 ¹H NMR (400 MHz, CD₃OD) δ 8.22 (s, 1H), 7.20-7.24 (m, 1H), 7.01 (t, 1H), 5.38 (s, 1H), 5.25 (s, 1H), 4.36- 4.54 (m, 3H), 4.14-4.28 (m, 4H), 3.89-3.98 (m, 2H), 3.75-3.80 (m, 1H), 3.63-3.67 (m, 1H), 3.37-3.50 (m, 2H), 3.12-3.23 (m, 2H), 3.01-3.07 (m, 1H), 2.56-2.66 (m, 1H), 2.20-2.36 (m, 3H), 2.10-2.16 (m, 1H), 1.87- 2.02 (m, 3H), 1.46 (t, 3H), 1.30 (t, 3H). |
| 780a | 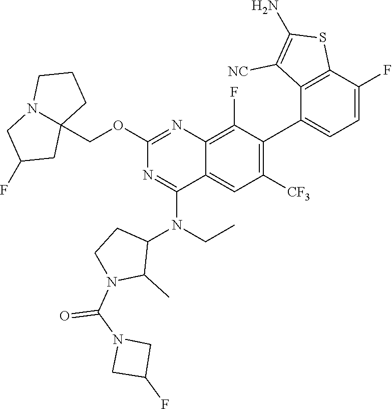 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2CC(F)C2)C1C] | |
| 781 | 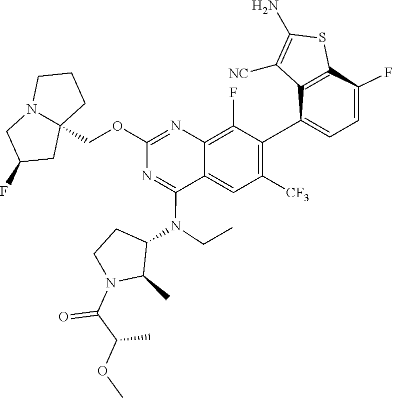 | LCMS m/z [M − 1]: 776.3 ¹H NMR (400 MHz, CD₃OD) δ 8.22 (s, 1H), 7.20-7.23 (m, 1H), 7.00 (t, 1H), 5.37 (s, 0.5H), 5.23 (s, 0.5H), 4.51-4.75 (m, 2H), 4.30-4.33 (m, 1H), 4.05-4.24 (m, 3H), 3.80-3.84 (m, 2H), 3.57-3.64 (m, 1H), 3.35 (s, 3H), 3.21-3.27 (m, 3H), 2.99-3.03 (m, 1H), 2.44-2.49 (m, 2H), 2.12-2.34 (m, 3H), 1.88-2.02 (m, 3H), 1.38-1.49 (m, 3H), 1.22-1.36 (m, 6H). |
| 781a | 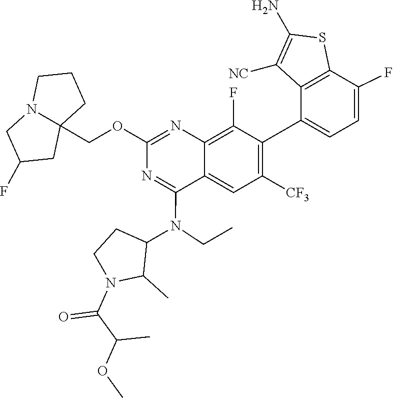 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C(C)OC)C1C] | |
| 782 | 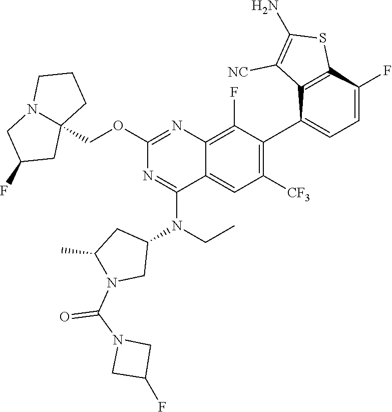 | LCMS m/z [M − 1]: 791.2 ¹H NMR (400 MHz, CD₃OD) δ 8.25 (s, 1H), 7.23-7.20 (m, 1H), 7.01 (t, 1H), 5.38-5.36 (m, 1H), 5.25-5.21 (m, 1H), 4.82-4.79 (m, 1H), 4.44-4.36 (m, 1H), 4.33- 4.25 (m, 2H), 4.20-4.15 (m, 1H), 4.14-4.10 (m, 1H), 4.04-3.98 (m, 1H), 3.95-3.83 (m, 4H), 3.45 (t, 1H), 3.26-3.18 (m, 2H), 3.06-2.99 (m, 1H), 2.56-2.50 (m, 1H), 2.35-1.97 (m, 7H), 1.95-1.88 (m, 1H), 1.46 (t, 3H), 1.37 (d, 3H). |
| 782a | 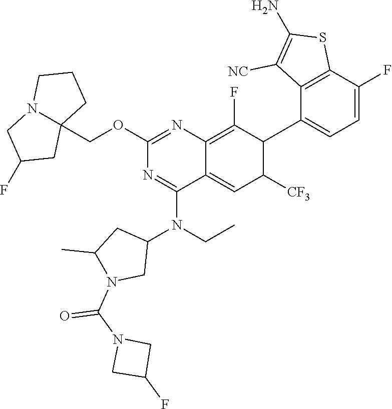 | |
| 783 | 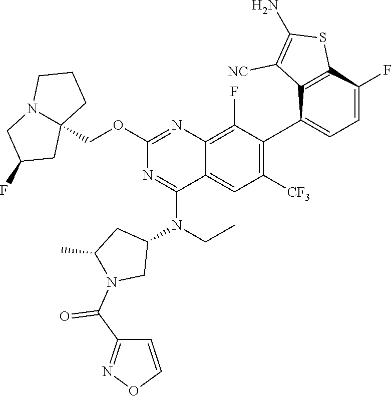 | LCMS m/z [M − 1]: 785.1 ¹H NMR (400 MHz, CD₃OD) δ 8.82-8.80 (m, 1H), 8.30-8.25 (m, 1H), 7.24-7.19 (m, 1H), 7.03-6.99 (m, 1H), 6.81-6.77 (m, 1H), 5.45 (s, 0.5H), 5.32 (s, 0.5H), 4.94-4.89 (m, 1H), 4.58-4.53 (m, 1H), 4.52-4.47 (m, 1H), 4.41-4.27 (m, 2H), 3.96-3.86 (m, 3H), 3.56- 3.48 (m, 2H), 3.23-3.16 (m, 1H), 2.72-2.65 (m, 1H), 2.45-2.31 (m, 2H), 2.29-2.04 (m, 5H), 2.03-1.95 (m, 1H), 1.59-1.42 (m, 6H). |
| 783a | 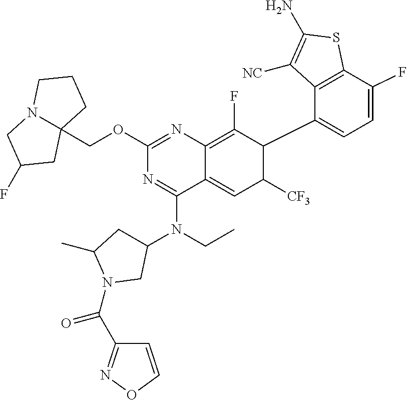 [SMILES: CCN(c1nc(OCC23CCCN2CC(F)C3)nc2c1=CC(C(F)(F)F)C(c1ccc(F)c3sc(N)c(C#N)c13)C=2F)C1CC(C)N(C(=O)c2ccon2)C1] | |
| 784 | 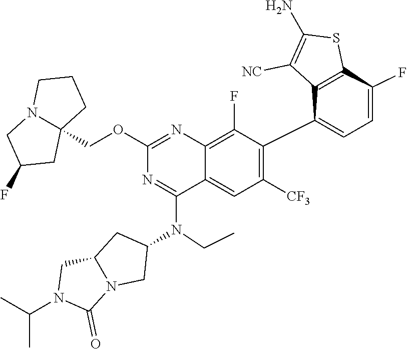 | LCMS m/z [M − 1]: 773.2 ¹H NMR (400 MHz, CD₃OD) δ 8.19 (s, 1H), 7.24-7.18 (m, 1H), 7.01 (t, 1H), 5.39-5.34 (m, 0.5H), 5.30-5.21 (m, 1.5H), 4.32 (d, 1H), 4.22 (d, 1H), 4.16-4.08 (m, 1H), 3.93-3.83 (m, 1H), 3.81-3.66 (m, 2H), 3.63-3.50 (m, 3H), 3.43 (d, 1H), 3.28-3.14 (m, 3H), 3.04-2.97 (m, 1H), 2.40-2.20 (m, 3H), 2.18-2.12 (m, 1H), 2.03-1.94 (m, 1H) 1.94-1.84 (m, 2H), 1.36 (t, 3H), 1.20 (dq, 6H). |
| 784a | 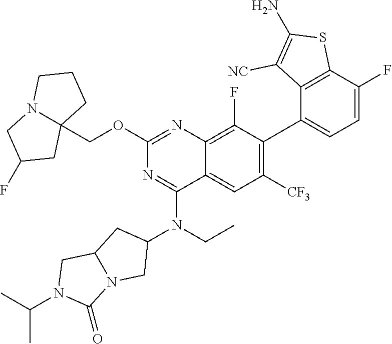 [SMILES: CCN(c1nc(OCC23CCCN2CC(F)C3)nc2c(F)c(-c3ccc(F)c4sc(N)c(C#N)c34)c(C(F)(F)F)cc12)C1CC2CN(C(C)C)C(=O)N2C1] | |
| 785 | 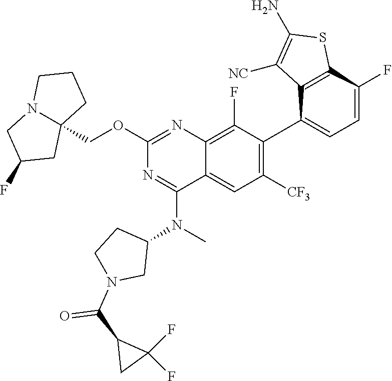 | LCMS m/z [M − 1]: 766.2 ¹H NMR (400 MHz, CD₃OD) δ 8.30 (d, 1H), 7.25-7.17 (m, 1H), 7.01 (t, 1H), 5.38-5.35 (m, 0.5H), 5.35-5.26 (m, 1H), 5.25-5.21 (m, 0.5H), 4.36-4.22 (m, 2H), 4.22- 4.17 (m, 0.5H), 4.06-4.00 (m, 0.5H), 3.97-3.90 (m, 0.5H), 3.87-3.75 (m, 1.5H), 3.63-3.50 (m, 1H), 3.49- 3.43 (m, 3H), 3.34-3.31 (m, 1H), 3.27-3.21 (m, 2H), 3.06-2.98 (m, 1H), 2.93-2.82 (m, 1H), 2.52-2.36 (m, 2H), 2.34-1.95 (m, 6H), 1.92-1.75 (m, 2H). |
| 785a | 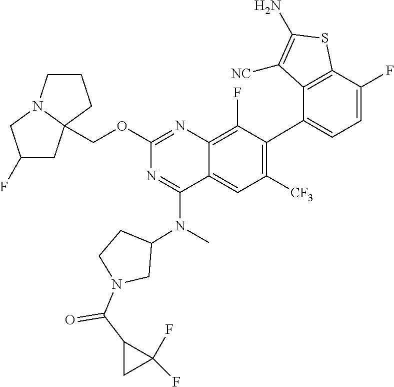 [SMILES: CN(c1nc(OCC23CCCN2CC(F)C3)nc2c(F)c(-c3ccc(F)c4sc(N)c(C#N)c34)c(C(F)(F)F)cc12)C1CCN(C(=O)C2CC2(F)F)C1] | |
| 786 | 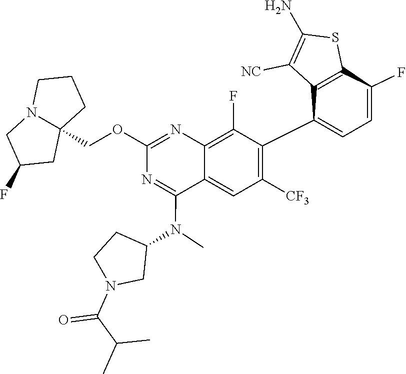 | LCMS m/z [M − 1]: 732.1 ¹H NMR (400 MHz, CD₃OD) δ 8.30 (d, 1H), 7.24-7.19 (m, 1H), 7.00 (t, 1H), 5.41-5.36 (m, 0.5H), 5.35-5.22 (m, 1.5H), 4.37-4.22 (m, 2H), 4.18-3.97 (m, 1H), 3.95- 3.77 (m, 1H), 3.72-3.62 (m, 1H), 3.58-3.32 (m, 2H), 3.45 (d, 3H), 3.28-3.22 (m, 2H), 3.10-3.00 (m, 1H), 2.88-2.76 (m, 1H), 2.49-2.42 (m, 1H), 2.40-2.28 (m, 2H), 2.27-2.12 (m, 2H), 2.06-1.96 (m, 2H), 1.95-1.85 (m, 1H), 1.16-1.11 (m, 6H). |
| 786a | 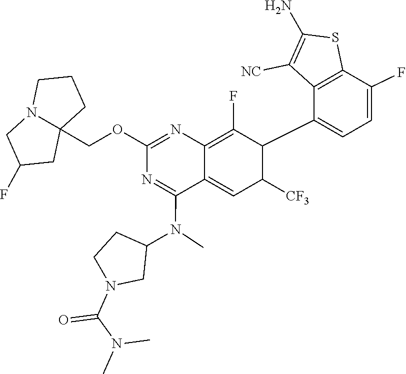 [SMILES: CN(C)C(=O)N1CCC(N(C)c2nc(OCC34CCCN3CC(F)C4)nc3c2=CC(C(F)(F)F)C(c2ccc(F)c4sc(N)c(C#N)c24)C=3F)C1] | |
| 787 | 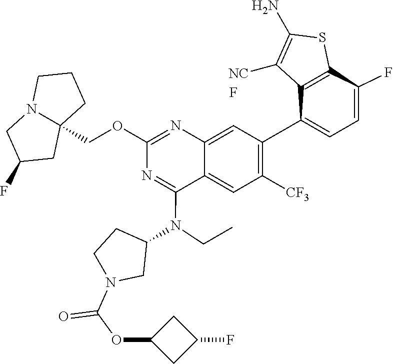 | LCMS m/z [M − 1]: 792.2 ¹H NMR (400 MHz, CD₃OD) δ 8.22 (s, 1H), 7.24-7.18 (m, 1H), 7.04-6.98 (t, 1H), 5.39-5.34 (m, 0.5H), 5.33- 5.27 (m, 0.5H), 5.25-5.21 (m, 0.5H), 5.20-5.10 (m, 1.5H), 5.06-4.97 (m, 1H), 4.34-4.20 (dq, 2H), 3.97-3.69 (m, 4H), 3.60-3.52 (m, 1H), 3.48-3.40 (m, 1H), 3.27- 3.11 (m, 3H), 3.05-2.97 (m, 1H), 2.65-2.08 (m, 9H), 2.02-1.84 (m, 3H), 1.44 (t, 3H). |
| 788 | 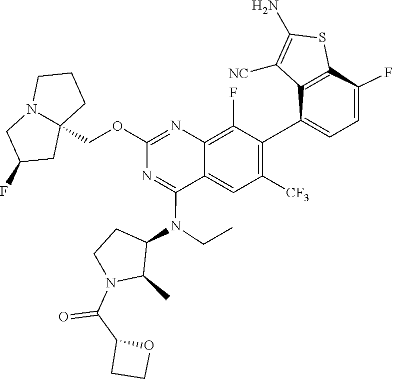 | LCMS m/z [M − 1]: 774.3 ¹H NMR (400 MHz, CD₃OD) δ 8.30 (s, 1H), 7.2269- 7.19 (m, 1H), 7.01 (t, 1H), 5.46-5.39 (m, 1H), 5.31-5.17 (m, 1H), 5.04-4.94 (m, 1H), 4.80-4.66 (m, 2H), 4.64- 4.58 (m, 1H), 4.37-4.20 (m, 2H), 4.10-3.98 (m, 1H), 3.93-3.84 (m, 1H), 3.80-3.68 (m, 1H), 3.63-3.52 (m, 1H), 3.44-3.37 (m, 1H), 3.09-3.01 (m, 1H), 2.98-2.79 (m, 2H), 2.64-2.52 (m, 1H), 2.49-2.34 (m, 2H), 2.29- 2.21 (m, 1H), 2.17-2.11 (m, 1H), 2.05-1.89 (m, 3H), 1.46-1.24 (m, 6H), 1.03-0.94 (m, 2H). |
| 788a | 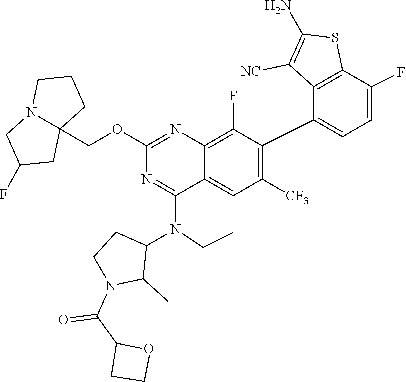 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CCO2)C1C] | |
| 789 | 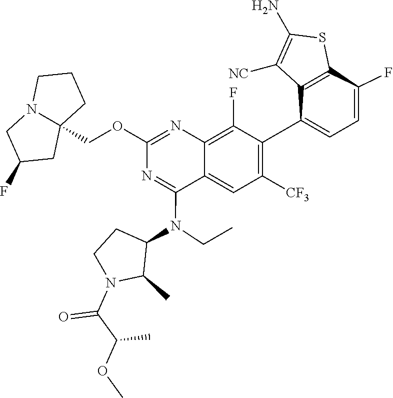 | LCMS m/z [M − 1]: 776.2 ¹H NMR (400 MHz, CD₃OD) δ 8.36-8.27 (m, 1H), 7.26-7.20 (m, 1H), 7.02 (t, 1H), 5.47 (s, 0.5H), 5.33 (s, 0.5H), 5.14-5.04 (m, 1H), 4.76-4.68 (m, 1H), 4.56-4.46 (m, 1H), 4.42-4.35 (m, 1H), 4.21-4.13 (m, 1H), 4.10- 4.03 (m, 1H), 3.97-3.81 (m, 2H), 3.78-3.70 (m, 1H), 3.63-3.49 (m, 2H), 3.37-3.32 (m, 3H), 3.23-3.14 (m, 1H), 2.70-2.58 (m, 1H), 2.53-2.34 (m, 3H), 2.26-1.95 (m, 5H), 1.45-1.32 (m, 6H), 1.13-1.07 (m, 1H), 1.00- 0.89 (m, 2H). |
| 790 | 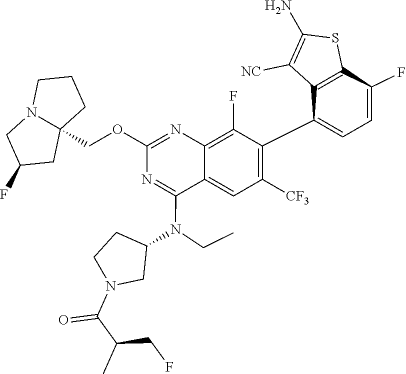 | LCMS m/z [M − 1]: 764.1 ¹H NMR (400 MHz, CD₃OD) δ = 8.28 (d, J = 12.2, 1H), 7.27-7.19 (m, 1H), 7.07-6.98 (m, 1H), 5.50 (s, 0.5H), 5.37 (s, 0.5H), 5.08 (d, J = 35.3, 3H), 4.69-4.23 (m, 5H), 3.96 (dd, J = 48.8, 9.8, 3H), 3.76-3.55 (m, 4H), 2.39 (dd, J = 43.1, 32.4, 5H), 2.10 (d, J = 55.3, 4H), 1.56- 1.36 (m, 3H), 1.14 (dt, J = 30.2, 7.1, 3H). |
| 791 | 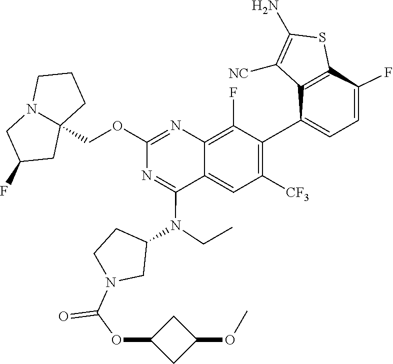 | LCMS m/z [M − 1]: 804.3 ¹H NMR (400 MHz, CD₃OD) δ 8.23 (s, 1H), 7.23- 7.20 (m, 1H), 7.01-6.99 (m, 1H), 5.38 (s, 0.5H), 5.24 (s, 0.5H), 5.08-5.04 (m, 2H), 4.64-4.60 (m, 1H), 4.30-4.24 (m, 2H), 3.96-3.72 (m, 4H), 3.58-3.31 (m, 3H), 3.23- 3.15 (m, 5H), 3.03 (m, 1H), 2.80-2.74 (m, 2H), 2.50- 2.35 (m, 5H), 2.21-1.99 (m, 5H), 1.48-1.44 (m, 3H). |
| 791a | 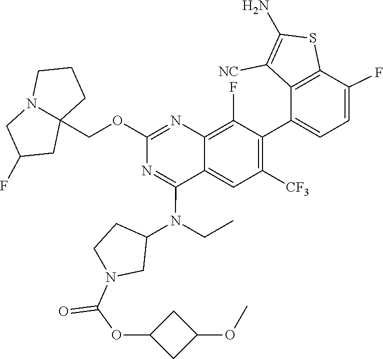 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2CC(OC)C2)C1] | |
| 792 | 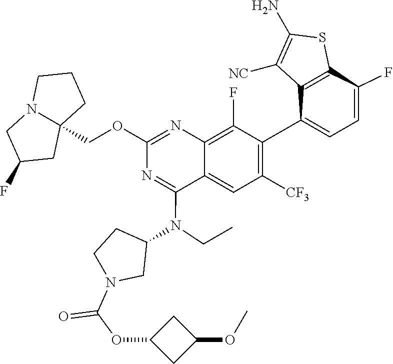 | LCMS m/z [M − 1]: 804.3 ¹H NMR (400 MHz, CD₃OD) δ 8.23 (s, 1H), 7.23- 7.20 (m, 1H), 7.03-6.99 (m, 1H), 5.38 (s, 0.5H), 5.24 (s, 0.5H), 5.08-5.04 (m, 2H), 4.30-4.24 (m, 2H), 4.08 (s, 1H), 3.96-3.72 (m, 4H), 3.58-3.31 (m, 3H), 3.23-3.15 (m, 5H), 3.03 (m, 1H), 2.45-1.88 (m, 12H), 1.43-1.28 (m, 3H). |
| 793 | 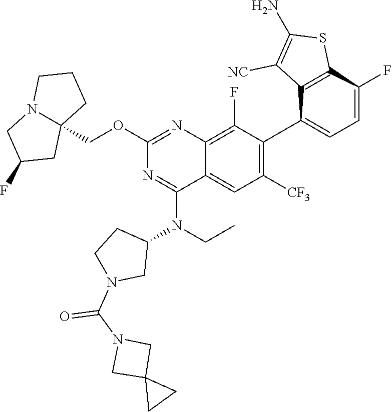 | LCMS m/z [M − 1]: 785.3 ¹H NMR (400 MHz, CD₃OD) δ 8.24 (s, 1H), 7.25- 7.18 (m, 1H), 7.05-6.97 (m, 1H), 5.39 (s, 0.5H), 5.25 (s, 0.5H), 5.03-4.94 (m, 2H), 4.35-4.22 (m, 2H), 4.15 (d, J = 7.8, 2H), 4.06 (d, J = 7.8, 2H), 3.95-3.79 (m, 3H), 3.77-3.66 (m, 1H), 3.63-3.54 (m, 1H), 3.53- 3.45 (m, 2H), 3.27-3.17 (m, 3H), 3.09-2.98 (m, 1H), 2.52-2.42 (m, 1H), 2.39-2.20 (m, 3H), 2.19- 2.11 (m, 1H), 2.07-1.96 (m, 2H), 1.94-1.85 (m, 1H), 1.46 (t, J = 6.9, 3H), 0.65 (s, 4H). |
| 793a | 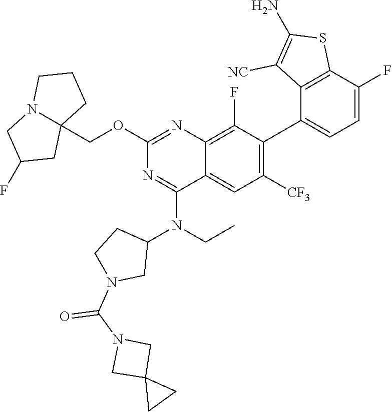 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2CC3(CC3)C2)C1] | |
| 794 | 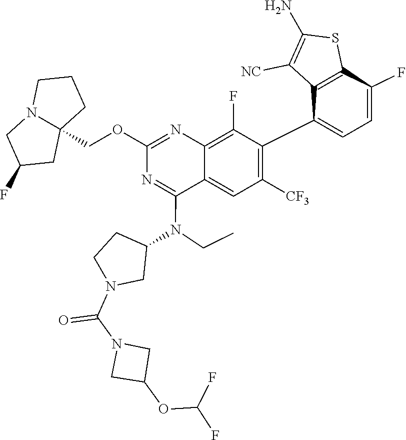 | LCMS m/z [M − 1]: 825.2 ¹H NMR (400 MHz, CD₃OD) δ 8.24 (s, 1H), 7.28- 7.15 (m, 1H), 7.09-6.94 (m, 1H), 6.45 (t, J = 74.1, 1H), 5.39 (s, 0.5H), 5.25 (s, 0.5H), 5.04-4.92 (m, 2H), 4.43- 4.20 (m, 3H), 4.14-4.04 (m, 1H), 4.04-3.95 (m, 1H), 3.95-3.78 (m, 3H), 3.76-3.64 (m, 1H), 3.63- 3.52 (m, 1H), 3.52-3.39 (m, 1H), 3.31-3.20 (m, 4H), 3.10-2.97 (m, 1H), 2.55-2.09 (m, 5H), 2.08-1.81 (m, 3H), 1.46 (t, J = 6.9, 3H). |
| 794a | 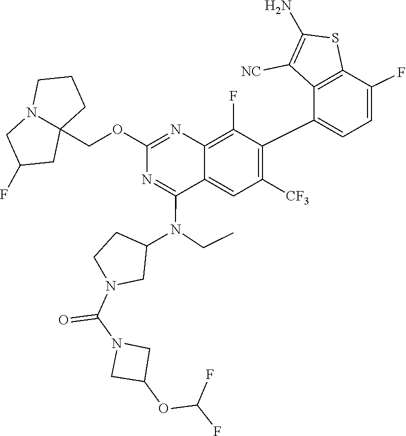 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2CC(OC(F)F)C2)C1] | |
| 795 | 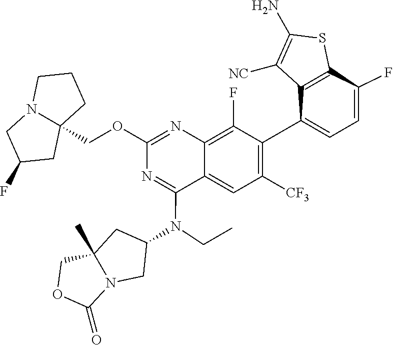 | LCMS m/z [M − 1]: 746.1 ¹H NMR (400 MHz, CD₃OD) δ 8.17 (s, 1H), 7.23-7.18 (m, 1H), 7.01 (t, 1H), 5.39 (s, 0.5H), 5.26 (s, 0.5H), 5.13-5.07 (m, 1H), 4.59 (s, 1H), 4.51 (d, 1H), 4.36-4.31 (m, 1H), 4.29-4.22 (m, 2H), 3.89-3.81 (m, 2H), 3.28- 3.21 (m, 2H), 3.10-3.03 (m, 1H), 2.62-2.56 (m, 1H), 2.51-2.39 (m, 1H), 2.35-2.29 (m, 1H), 2.25-2.18 (m, 2H), 2.07-1.99 (m, 2H), 1.94-1.88 (m, 1H), 1.50-1.39 (m, 6H). |
| 795a | 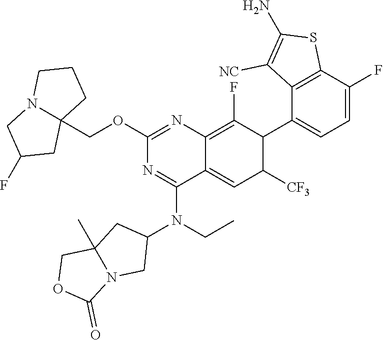 [SMILES: CCN(c1nc(OCC23CCCN2CC(F)C3)nc2c1=CC(C(F)(F)F)C(c1ccc(F)c3sc(N)c(C#N)c13)C=2F)C1CN2C(=O)OCC2(C)C1] | |
| 796 | 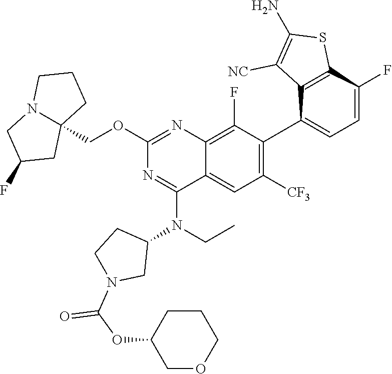 | LCMS m/z [M − 1]: 804.2 ¹H NMR (400 MHz, CD₃OD) δ 8.23 (s, 1H), 7.25-7.18 (m, 1H), 7.01 (t, 1H), 5.36 (s, 0.5H), 5.23 (s, 0.5H), 5.03 (s, 1H), 4.66 (s, 1H), 4.32-4.20 (m, 2H), 3.98-3.83 (m, 3H), 3.80-3.72 (m, 2H), 3.66-3.56 (m, 4H), 3.48- 3.40 (m, 1H), 3.26-3.15 (m, 3H), 3.04-2.98 (m, 1H), 2.51-2.43 (m, 1H), 2.37-2.32 (m, 1H), 2.28-2.07 (m, 3H), 2.03-1.94 (m, 3H), 1.90-1.76 (m, 3H), 1.57 (s, 1H), 1.45 (t, 3H). |
| 796a | 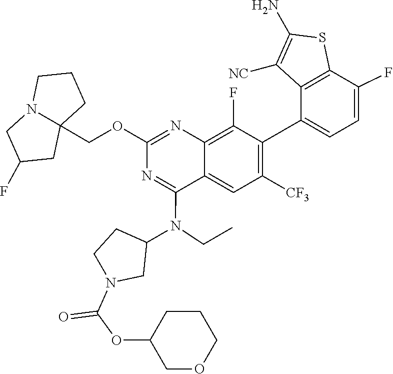 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2CCCOC2)C1] | |
| 797 | 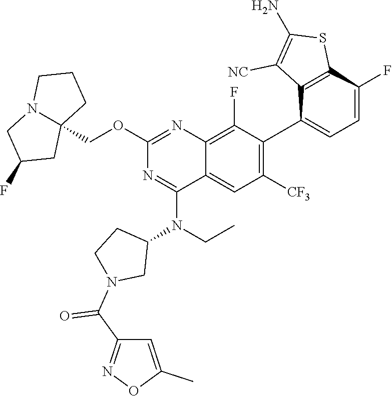 | LCMS m/z [M − 1]: 784.3 ¹H NMR (400 MHz, CD₃OD) δ = 8.25 (s, 1H), 7.27- 7.16 (m, 1H), 7.01 (t, J = 8.9, 1H), 6.46 (s, 1H), 5.35 (s, 1H), 5.22 (s, 1H), 5.11-4.97 (m, 1H), 4.60-4.42 (m, 1H), 4.39-4.12 (m, 3H), 4.11-3.76 (m, 4H), 3.73- 3.58 (m, 8.9, 1H), 3.26-3.14 (m, 2H), 3.08-2.95 (m, 1H), 2.66-2.05 (m, 8H), 2.04-1.80 (m, 3H), 1.49 (t, J = 6.9, 3H). |
| 797a | 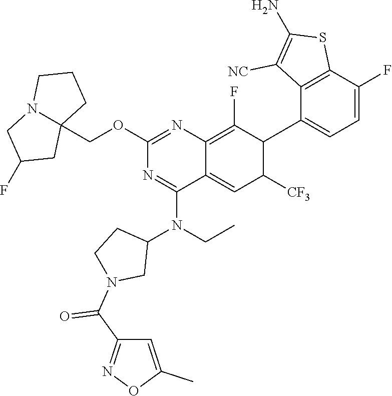 | |
| 798 | 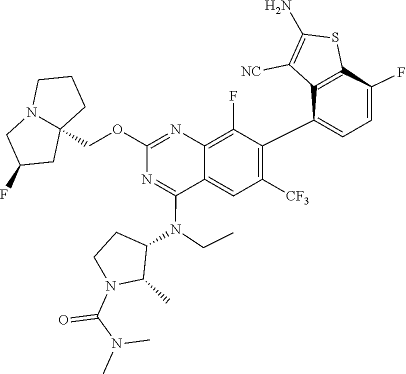 | LCMS m/z [M − 1]: 761.3 ¹H NMR (400 MHz, CD₃OD) δ 8.28 (s, 1H), 7.23-7.20 (m, 1H), 7.01 (t, 1H), 5.43 (s, 0.5H), 5.29 (s, 0.5H), 4.59 (s, 1H), 4.40 (s, 1H), 4.08-4.04 (m, 1H), 3.92-3.85 (m, 2H), 3.59-3.44 (m, 2H), 3.38-3.35 (m, 2H), 3.15- 3.03 (m, 2H), 2.90 (s, 6H), 2.49-2.41 (m, 2H), 2.35-2.21 (m, 3H), 2.07-1.95 (m, 3H), 1.43 (t, 3H), 1.05 (d, 3H). |
| 798a | 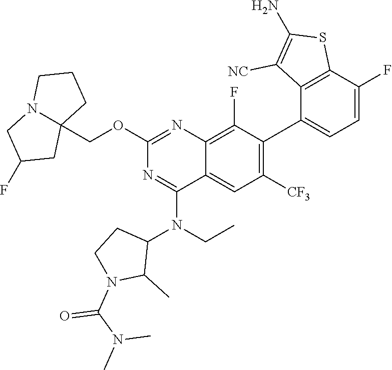 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N(C)C)C1C] | |
| 799 | 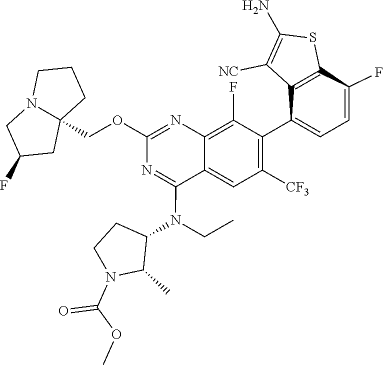 | LCMS m/z [M − 1]: 748.1 ¹H NMR (400 MHz, CD₃OD) δ 8.31 (s, 1H), 7.23-7.20 (m, 1H), 7.01 (t, 1H), 5.38 (s, 0.5H), 5.25 (s, 0.5H), 4.81-4.71 (m, 2H), 4.33-4.21 (m, 2H0, 4.09-3.86 (m, 3H), 3.71 (s, 3H), 3.50-3.37 (m, 2H), 3.25-3.19 (m, 2H), 3.06-3.00 (m, 1H), 2.55-2.35 (m, 3H), 2.28-2.11 (m, 3H), 2.04-1.96 (m, 2H), 1.47-1.37 (m, 3H), 1.47 (d, 3H). |
| 799a | 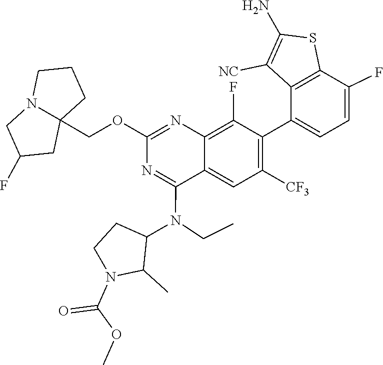 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC)C1C] | |
| 800 | 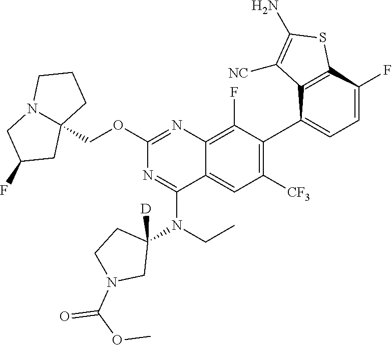 | LCMS m/z [M − 1]: 735.2. ¹H NMR (400 MHz, CD₃OD) δ 8.22 (s, 1H), 7.23-7.20 (m, 1H), 7.01 (t, J = 8.8 Hz, 1H), 5.378-5.24 (d, J = 54.0 Hz, 1H), 4.31-4.23 (m, 2H), 3.92-3.85 (m, 4H), 3.72 (s, 3H), 3.57 (d, J = 10.8 Hz, 1H), 3.50-3.40 (m, 1H), 3.16- 3.21 (m, 3H), 3.95-2.95 (m, 1H), 2.50-1.85 (m, 8H), 1.45 (t, J = 7.2 Hz, 3H). |
| 800a | 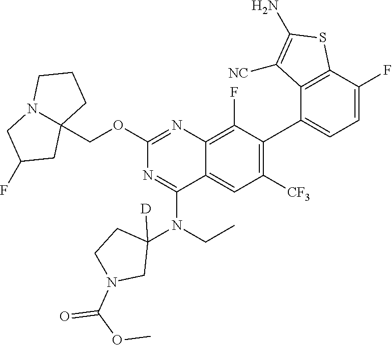 [SMILES: [2H]C1(N(CC)c2nc(OCC34CCCN3CC(F)C4)nc3c(F)c(-c4ccc(F)c5sc(N)c(C#N)c45)c(C(F)(F)F)cc23)CCN(C(=O)OC)C1] | |
| 801 | 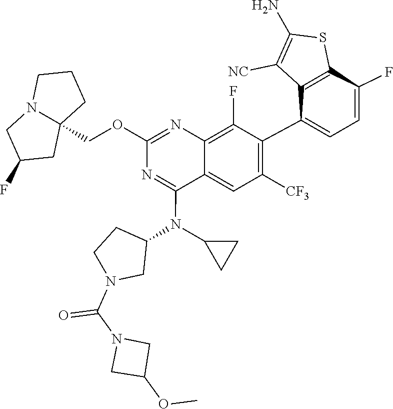 | LCMS m/z [M − 1]: 801.2 ¹H NMR (400 MHz, CD₃OD): δ 8.87 (s, 1 H), 7.17-7.26 (m, 1 H), 6.96-7.05 (m, 1 H), 5.38 (s, 0.5 H), 5.24 (s, 0.5 H), 4.73-4.83 (m, 1 H), 4.59 (s, 0.5 H), 4.12-4.30 (m, 4.5 H), 4.02-4.12 (m, 1 H), 3.80-3.96 (m, 2 H), 3.59- 3.76 (m, 2 H), 3.41-3.54 (m, 2 H), 3.18-3.28 (m, 3 H), 2.96-3.08 (m, 1H), 2.45-2.71 (m, 2 H), 2.14-2.32 (m, 2 H), 1.82-2.06 (m, 3 H), 0.94-1.09 (m, 2 H), 0.63-0.80 (m, 2 H). |
| 801a | 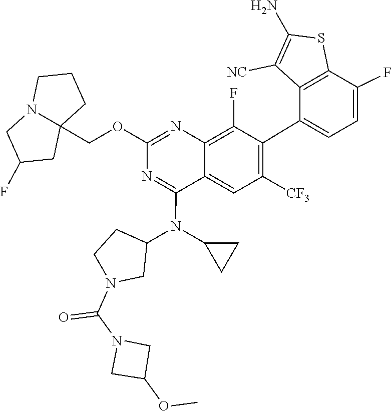 [SMILES: COC1CN(C(=O)N2CCC(N(c3nc(OCC45CCCN4CC(F)C5)nc4c(F)c(-c5ccc(F)c6sc(N)c(C#N)c56)c(C(F)(F)F)cc34)C3CC3)C2)C1] | |
| 802 | 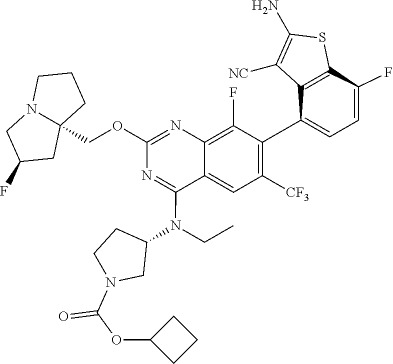 | LCMS m/z [M − 1]: 774.2 ¹H NMR (400 MHz, CD₃OD) δ 8.22 (s, 1H), 7.20-7.23 (m, 1H), 6.98-7.03 (m, 1H), 5.36 (s, 0.5H), 5.23 (s, 0.5H), 4.93-4.97 (m, 2H), 4.22-4.26 (m, 2H), 3.75-3.85 (m, 3H), 3.71-3.78 (m, 1H), 3.54-3.58 (m, 1H), 3.39- 3.47 (m, 1H), 3.14-3.22 (m, 3H), 2.97-3.01 (m, 1H), 2.19-2.47 (m, 6H), 2.09-2.15 (m, 3H), 1.94-2.00 (m, 2H), 1.79-1.89 (m, 2H), 1.60-1.67 (m, 1H), 1.45 (t, 3H). |
| 802a | 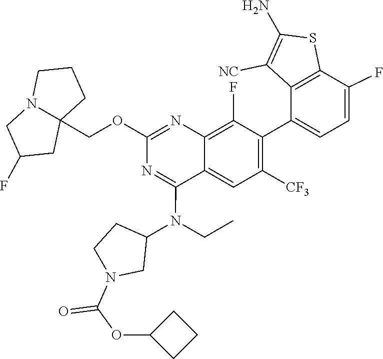 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2CCC2)C1] | |
| 803 | 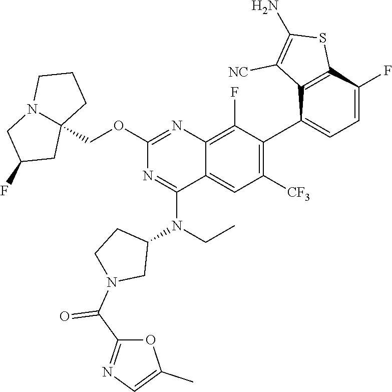 | LCMS m/z [M − 1]: 785.3 ¹H NMR (400 MHz, CD₃OD) δ = 8.26 (s, 2H), 7.28- 7.18 (m, 1H), 7.08-6.93 (m, 2H), 5.37 (s, 0.5H), 5.23 (s, 0.5H), 5.13-4.96 (m, 1H), 4.74-4.64 (m, 1H), 4.63- 4.51 (m, 1H), 4.40-4.00 (m, 4H), 3.98-3.80 (m, 2H), 3.66 (d, J = 20.6, 1H), 3.29-3.18 (m, 2H), 3.09- 2.97 (m, 1H), 2.69-2.34 (m, 5H), 2.31-2.06 (m, 3H), 2.06-1.82 (m, 3H), 1.59-1.43 (m, 3H). |
| 803a | 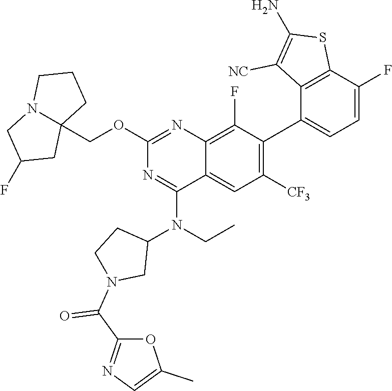 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2ncc(C)o2)C1] | |
| 804 | 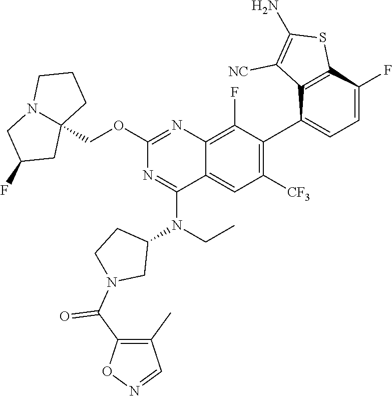 | LCMS m/z [M − 1]: 785.2 ¹H NMR (400 MHz, CD₃OD) δ 8.39 (d, 1H), 8.26 (s, 1H), 7.22 (t, 1H), 7.01 (t, 1H), 5.34 (s, 0.5H), 5.21 (s, 0.5H), 5.03-5.08 (m, 1H), 4.40-4.45 (m, 1H), 4.16-4.24 (m, 3H), 3.80-4.03 (m, 4H), 3.63-3.70 (m, 1H), 3.15- 3.24 (m, 2H), 3.01 (s, 1H), 2.45-2.62 (m, 2H), 2.08-2.32 (m, 6H), 1.85-2.07 (m, 3H), 1.45-1.50 (m, 3H). |
| 804a | 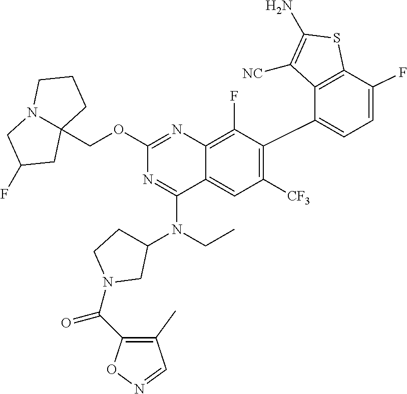 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2oncc2C)C1] | |
| 805 | 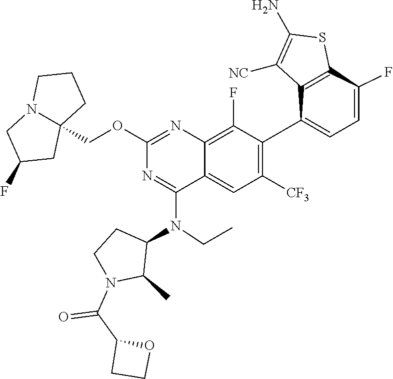 | LCMS m/z [M − 1]: 774.2 ¹H NMR (400 MHz, CD₃OD) δ 8.22 (s, 1H), 7.21-7.24 (m, 1H), 7.01 (t, 1H), 5.36-5.51 (m, 1.5H), 5.24 (s, 0.5H), 4.69-4.74 (m, 1H), 4.53-4.62 (m, 3H), 4.17-4.28 (m, 2H), 3.75-3.90 (m, 2H), 3.75-3.71 (m, 3H), 3.13- 3.26 (m, 2H), 2.79-3.02 (m, 3H), 2.46-2.57 (m, 1H), 2.21-2.43 (m, 3H), 2.10-2.12 (m, 1H), 1.86-2.03 (m, 3H), 1.31-1.45 (m, 6H). |
| 806 | 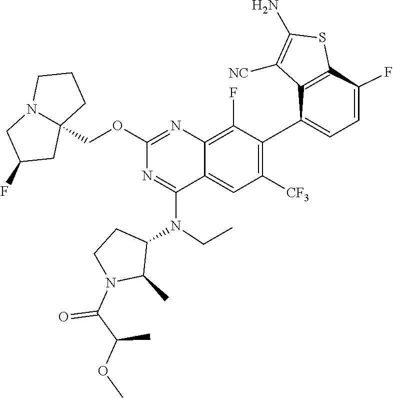 | LCMS m/z [M − 1]: 776.3 ¹H NMR (400 MHz, CD₃OD) δ 8.21 (s, 1H), 7.20-7.24 (m, 1H), 7.01 (t, 1H), 5.37 (s, 0.5H), 5.23 (s, 0.5H), 4.70-4.76 (m, 1H), 4.46-4.54 (m, 1H), 4.14-4.34 (m, 3H), 3.92-4.08 (m, 1H), 3.50-3.84 (m, 3H), 3.33-3.35 (m, 3H), 3.18-3.27 (m, 3H), 2.99-3.04 (m, 1H), 2.44- 2.49 (m, 2H), 2.11-2.36 (m, 3H), 1.86-2.02 (m, 3H), 1.29-1.39 (m, 9H). |
| 807 | 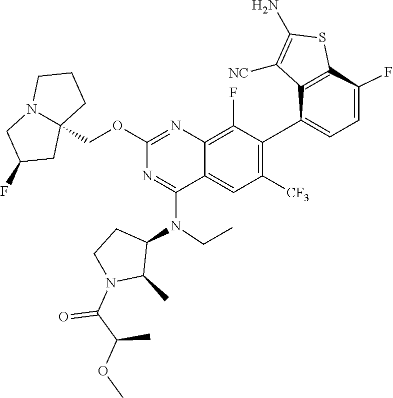 | LCMS m/z [M − 1]: 776.2 ¹H NMR (400 MHz, CD₃OD) δ 8.30 (s, 1H), 7.26-7.20 (m, 1H), 7.01 (t, 1H), 5.37 (s, 0.5H), 5.23 (s, 0.5H), 5.04-4.98 (m, 1H), 4.76-4.67 (m, 1H), 4.59 (s, 1H), 4.35-4.15 (m, 3H), 4.07-4.84 (m, 3H), 3.68-3.61 (m, 1H), 3.36-3.32 (m, 3H), 3.25-3.18 (m, 2H), 3.04-2.97 (m, 1H), 2.66-2.56 (m, 1H), 2.51-2.45 (m, 1H), 2.35- 2.25 (m, 2H), 2.10-1.94 (m, 3H), 1.91-1.84 (m, 1H), 1.40-1.29 (m, 6H), 1.11-0.93 (m, 3H). |
| 808 | 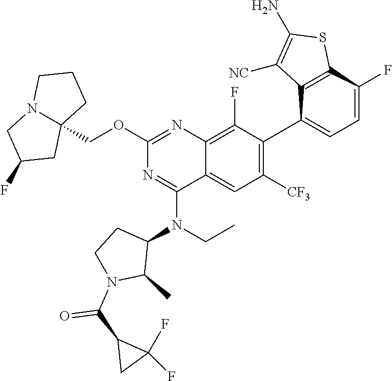 | LCMS m/z [M − 1]: 794.2 ¹H NMR (400 MHz, CD₃OD) δ 8.32-8.21 (m, 1H), 7.25-7.19 (m, 1H), 7.01 (t, 1H), 5.37 (s, 0.5H), 5.23 (s, 0.5H), 5.07-4.98 (m, 1H), 4.81-4.73 (m, 1H), 4.64-4.58 (m, 1H), 4.34-4.21 (m, 2H), 4.08-4.01 (m, 1H), 3.97- 3.89 (m, 1H), 3.85-3.77 (m, 1H), 3.69-3.56 (m, 1H), 3.25-3.19 (m, 2H), 3.01-2.84 (m, 2H), 2.67-2.49 (m, 2H), 2.39-2.21 (m, 2H), 2.11-1.97 (m, 3H), 1.86-1.78 (m, 1H), 1.55-1.24 (m, 6H), 1.10 (d, 1H), 1.01 (d, 1H). |
| 808a | 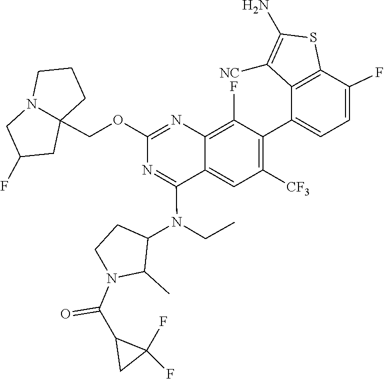 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CC2(F)F)C1C] | |
| 809 | 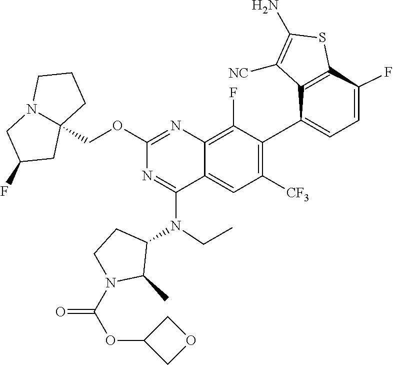 | LCMS m/z [M − 1]: 790.3. ¹H NMR (400 MHz, CD₃OD) δ 8.22 (s, 1H), 7.22 (t, 1H), 7.01 (t, 1H), 5.45-5.36 (m, 1.5H), 5.24 (s, 0.5H), 4.93-4.89 (m, 2H), 4.70-4.58 (m, 3H), 4.38-4.17 (m, 3H), 3.97-3.76 (m, 3H), 3.60-3.50 (m, 1H), 2.27-3.18 (m, 3H), 3.06-2.98 (m, 1H), 2.55-2.46 (m, 1H), 2.42- 2.20 (m, 3H), 2.16-2.08 (m, 1H), 2.05-1.95 (m, 2H), 1.92-1.85 (m, 1H), 1.49-1.32 (m, 6H). |
| 809a | 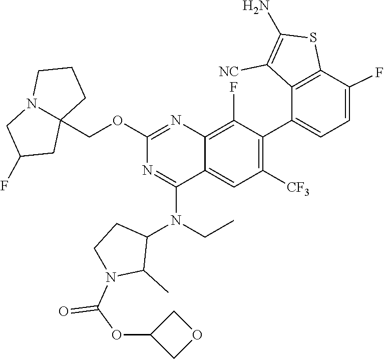 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2COC2)C1C] | |
| 810 | 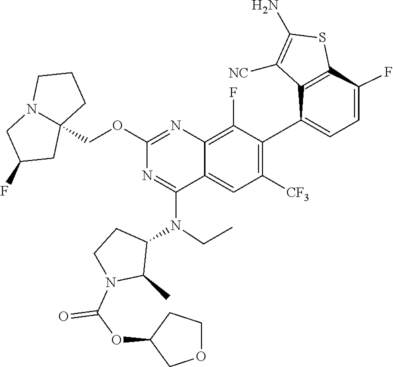 | LCMS m/z [M − 1]: 804.3 ¹H NMR (400 MHz, CD₃OD) δ 8.21 (s, 1H), 7.22 (t, 1H), 7.01 (t, 1H), 5.37 (s, 0.5H), 5.30-5.22 (m, 1.5H), 4.72-4.56 (m, 2H), 4.33-4.20 (m, 3H), 3.97-3.75 (m, 7H), 3.50-3.44 (m, 1H), 3.26-3.20 (m, 2H), 3.05-2.97 (m, 1H), 2.54-2.32 (m, 3H), 2.28-2.14 (m, 3H), 2.13- 1.95 (m, 4H), 1.91-1.84 (m, 1H), 1.42 (m, 3H), 1.36 (d, 3H). |
| 810a | 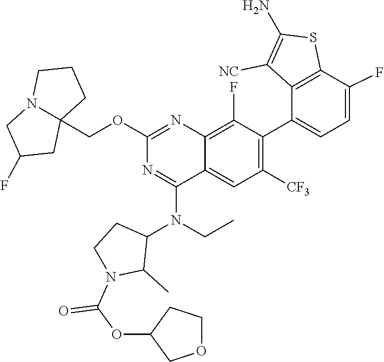 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2CCOC2)C1C] | |
| 811 | 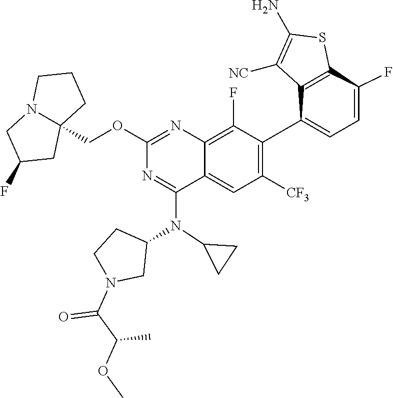 | LCMS m/z [M − 1]: 774.2 ¹H NMR (400 MHz, CD₃OD) δ = 8.70 (d, J = 4.4, 1H), 7.28-7.16 (m, 1H), 7.07-6.97 (m, 1H), 5.45 (s, 0.5H), 5.30 (d, J = 13.1, 0.5H), 4.93 (s, 1H), 4.59 (s, 1H), 4.48- 4.07 (m, 4H), 4.07-3.96 (m, 0.5H), 3.88 (s, 0.5H), 3.81- 3.71 (m, 1H), 3.70-3.34 (m, 6H), 3.13 (s, 1H), 2.81- 1.84 (m, 9H), 1.34 (dd, J = 14.1, 6.6, 3H), 1.06 (d, J = 7.0, 2H), 0.71 (s, 2H). |
| 811a | 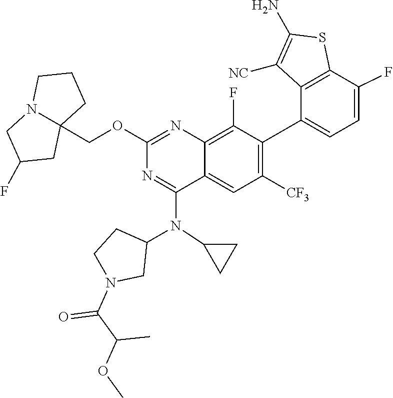 [SMILES: COC(C)C(=O)N1CCC(N(c2nc(OCC34CCCN3CC(F)C4)nc3c(F)c(-c4ccc(F)c5sc(N)c(C#N)c45)c(C(F)(F)F)cc23)C2CC2)C1] | |
| 812 | 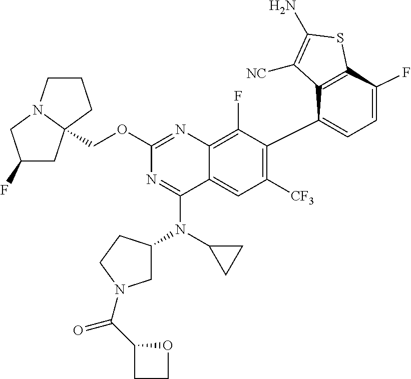 | LCMS m/z [M − 1]: 772.1 ¹H NMR (400 MHz, CD₃OD) δ = 8.72 (s, 1H), 7.23 (s, 1H), 7.08-6.95 (m, 1H), 5.44 (dd, J = 23.1, 7.4, 1H), 5.34 (s, 0.5H), 5.18 (s, 0.5H), 4.82-4.56 (m, 3H), 4.42 (dd, J = 26.5, 11.0, 1H), 4.32-4.18 (m, 1H), 4.04-3.92 (m, 1H), 3.84-3.45 (m, 5H), 3.13 (s, 2H), 3.05-1.76 (m, 11H), 1.14-0.96 (m, 2H), 0.73 (d, J = 4.2, 2H). |
| 812a | 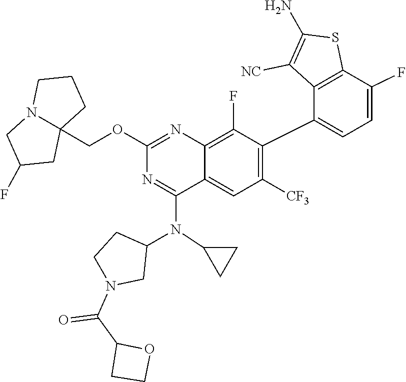 [SMILES: N#Cc1c(N)sc2c(F)ccc(-c3c(C(F)(F)F)cc4c(N(C5CC5)C5CCN(C(=O)C6CCO6)C5)nc(OCC56CCCN5CC(F)C6)nc4c3F)c12] | |
| 813 | 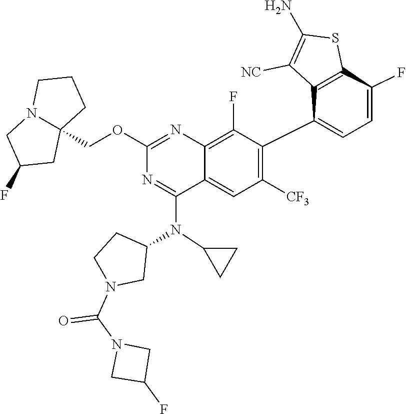 | LCMS m/z [M − 1]: 789.2 ¹H NMR (400 MHz, CD₃OD) δ = 8.71 (s, 1H), 7.23 (dd, J = 8.2, 4.9, 1H), 7.07-6.96 (m, 1H), 5.47 (s, 0.5H), 5.38 (s, 0.5H), 5.34 (s, 0.5H), 5.23 (s, 0.5H), 4.81 (d, J = 8.1, 1H), 4.60 (s, 1H), 4.47-4.24 (m, 3H), 4.07 (dd, J = 16.3, 8.4, 3H), 3.80-3.41 (m, 6H), 3.16 (d, J = 27.4, 2H), 2.71-1.90 (m, 8H), 1.07 (t, J = 16.7, 2H), 0.73 (s, 2H). |
| 813a | 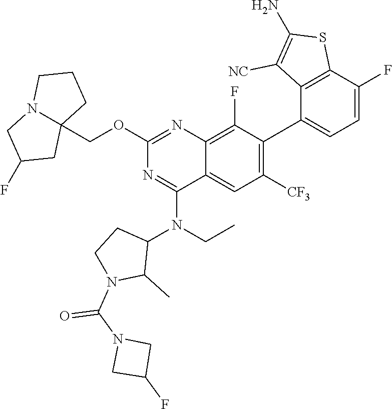 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2CC(F)C2)C1C] | |
| 814 | 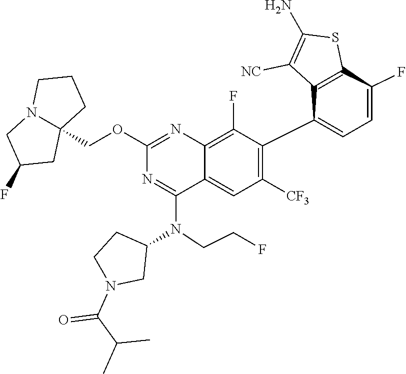 | LCMS m/z [M − 1]: 764.78 ¹H NMR (400 MHz, CD₃OD) δ 8.18-8.24 (m, 1H), 7.19-7.25 (m, 1H), 6.98-7.05 (m, 1H), 5.40 (s, 0.5H), 5.26 (s, 0.5H), 4.92-5.13 (m, 2H), 4.55-4.84 (m, 3H), 4.23-4.39 (m, 2H), 4.06-4.19 (m, 2H), 3.88-4.00 (m, 1H), 3.77-3.85 (m, 1H), 3.61-3.70 (m, 1H), 3.34-3.49 (m, 2H), 3.01-3.12 (m, 1H), 2.75-2.87 (m, 1H), 2.12- 2.50 (m, 5H), 1.88-2.08 (m, 3H), 1.06-1.19 (m, 6H). |
| 814a | 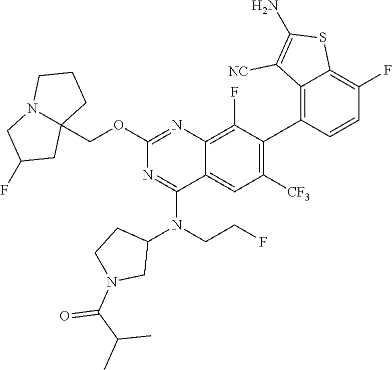 [SMILES: CC(C)C(=O)N1CCC(N(CCF)c2nc(OCC34CCCN3CC(F)C4)nc3c(F)c(-c4ccc(F)c5sc(N)c(C#N)c45)c(C(F)(F)F)cc23)C1] | |
| 815 | 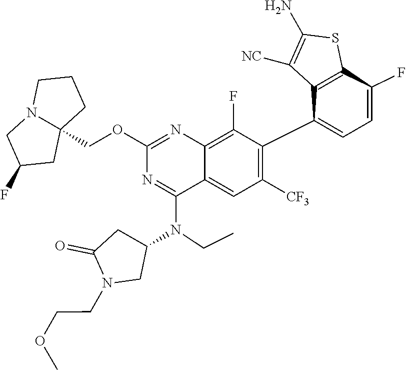 | LCMS m/z [M − 1]: 748.1 ¹H NMR (400 MHz, CD₃OD) δ 8.15 (s, 1H), 7.24-7.17 (m, 1H), 7.01 (t, 1H), 5.42-5.36 (m, 0.5H), 5.28-5.23 (m, 0.5H), 4.84-4.75 (m, 1H), 4.27-4.13 (m, 2H), 4.05- 3.97 (m, 1H), 3.91-3.76 (m, 3H), 3.27-3.55 (m, 3H), 3.47-3.40 (m, 1H), 3.34 (s, 3H), 3.28-3.11 (m, 3H), 3.07-2.94 (m, 2H), 2.90-2.81 (m, 1H), 2.41-2.26 (m, 1H), 2.25-2.10 (m, 2H), 2.03-1.86 (m, 3H), 1.56-1.45 (m, 3H). |
| 815a | 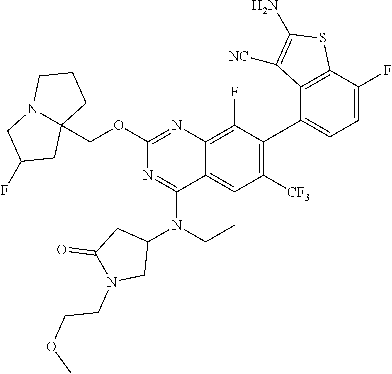 [SMILES: CCN(c1nc(OCC23CCCN2CC(F)C3)nc2c(F)c(-c3ccc(F)c4sc(N)c(C#N)c34)c(C(F)(F)F)cc12)C1CC(=O)N(CCOC)C1] | |
| 816 | 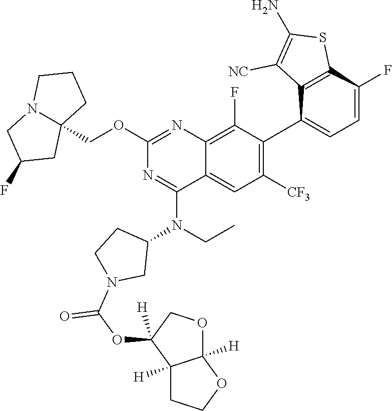 | LCMS m/z [M − 1]: 832.2 ¹H NMR (400 MHz, CD₃OD) δ 8.25-8.19 (m, 1H), 7.24-7.18 (m, 1H), 7.01 (t, 1H), 5.68 (t, 1H), 5.38 (s, 0.5H), 5.24 (s, 0.5H), 5.19-5.15 (m, 1H), 5.07-5.00 (m, 1H), 4.35-4.21 (m, 2H), 4.06-3.74 (m, 8H), 3.65-3.35 (m, 3H), 3.27-3.22 (m, 2H), 3.13-3.01 (m, 2H), 2.51- 2.27 (m, 3H), 2.24-2.07 (m, 3H), 2.02-1.86 (m, 4H), 1.48-1.40 (m, 3H). |
| 816a | 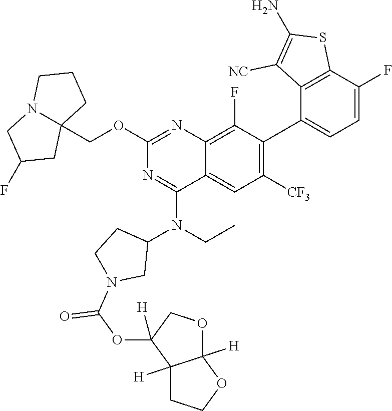 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2COC3OCCC23)C1] | |
| 817 | 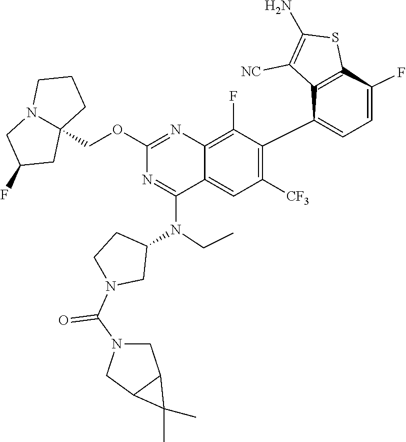 | LCMS m/z [M − 1]: 813.3 ¹H NMR (400 MHz, CD₃OD) δ 8.24 (s, 1H), 7.24-7.18 (m, 1H), 7.01 (t, 1H), 5.38-5.35 (m, 0.5H), 5.26-5.21 (m, 0.5H), 5.01-4.94 (m, 1H), 4.34-4.22 (m, 2H), 3.92- 3.78 (m, 3H), 3.71-3.57 (m, 4H), 3.54-3.46 (m, 2H), 3.43-3.39 (m, 1H), 3.28-3.16 (m, 3H), 3.06-2.99 (m, 1H), 2.43-2.20 (m, 4H), 2.17-2.10 (m, 1H), 2.04-1.95 (m, 2H), 1.93-1.85 (m, 1H), 1.45 (t, 3H), 1.42-1.38 (m, 2H), 1.04 (s, 3H), 0.92 (s, 3H). |
| 817a | 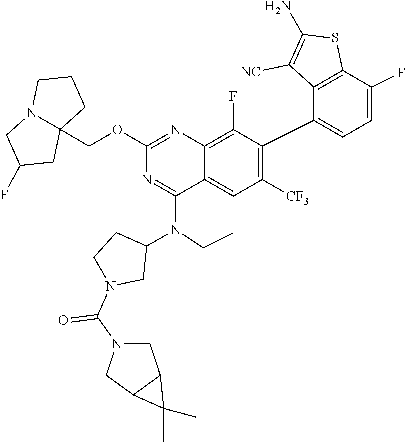 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2CC3C(C2)C3(C)C)C1] | |
| 818 | 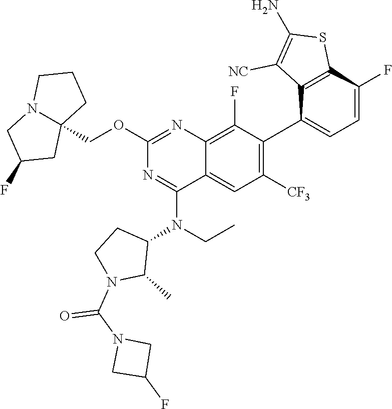 | LCMS m/z [M − 1]: 791.2 ¹H NMR (400 MHz, CD₃OD) δ 8.29 (s, 1H), 7.26-7.18 (m, 1H), 7.01 (t, 1H), 5.37 (s, 1H), 5.24 (s, 1H), 4.82- 4.71 (m, 2H), 4.43-4.20 (m, 4H), 4.18-3.97 (m, 3H), 3.95-3.86 (m, 1H), 3.70-3.63 (m, 1H), 3.48-3.41 (m, 1H), 3.26-3.18 (m, 3H), 3.07-2.99 (m, 1H), 2.56-2.48 (m, 1H), 2.46-2.38 (m, 1H), 2.33-2.14 (m, 3H), 2.04- 1.95 (m, 2H), 1.94-1.87 (m, 1H), 1.40 (t, 3H), 1.01 (d, 3H). |
| 818a | 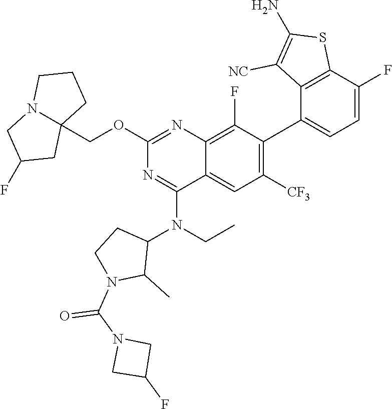 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2CC(F)C2)C1C] | |
| 820 | 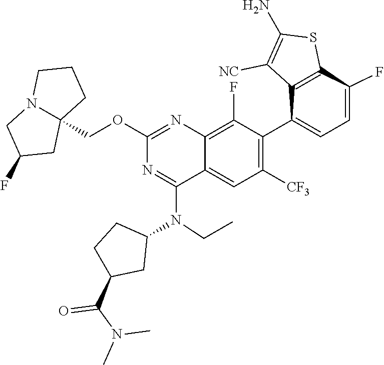 | LCMS m/z [M − 1]: 746.3 ¹H NMR (400 MHz, CD₃OD): δ 8.23 (s, 1 H), 7.19-7.23 (m, 1 H), 6.98-7.03 (m, 1 H), 5.40 (s, 0.5 H), 5.26 (s, 0.5 H), 4.98-5.03 (m, 1 H), 4.25-4.41 (m, 2 H), 3.74- 3.94 (m, 2 H), 3.36-3.55 (m, 2 H), 3.22-3.27 (m, 1 H), 3.01-3.18 (m, 4 H), 2.94 (s, 3 H), 1.60-1.46 (m, 13 H), 1.43-1.46 (m, 3 H). |
| 820a | 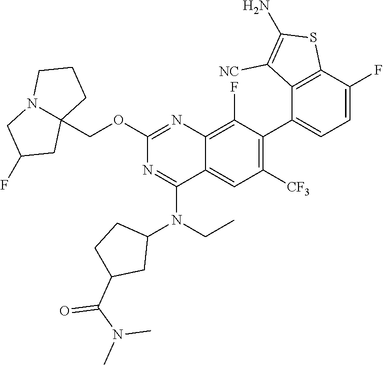 [SMILES: CCN(c1nc(OCC23CCCN2CC(F)C3)nc2c(F)c(-c3ccc(F)c4sc(N)c(C#N)c34)c(C(F)(F)F)cc12)C1CCC(C(=O)N(C)C)C1] | |
| 821 | 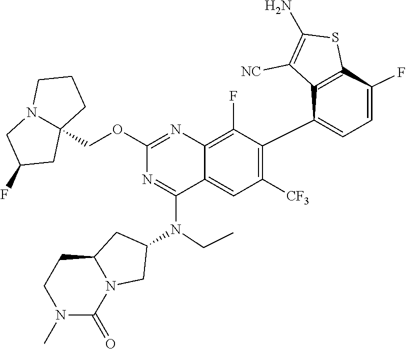 | LCMS m/z [M − 1]: 759.2 ¹H NMR (400 MHz, CD₃OD): δ 8.16-8.25 (m, 1 H), 7.18- 7.24 (m, 1 H), 6.97-7.04 (m, 1 H), 5.37 (s, 0.5 H), 5.23 (s, 0.5 H), 4.91-5.00 (m, 1 H), 4.08-4.32 (m, 3 H), 3.71- 4.02 (m, 3 H), 3.55-3.67 (m, 1 H), 3.39-3.49 (m, 1 H), 3.11-3.28 (m, 4 H), 2.98-3.08 (m, 1 H), 2.93 (s, 1 H), 2.13-2.66 (m, 6 H), 1.89-2.05 (m, 3 H), 1.58-1.72 (m, 1 H), 1.38-1.48 (m, 3 H). |
| 821a | 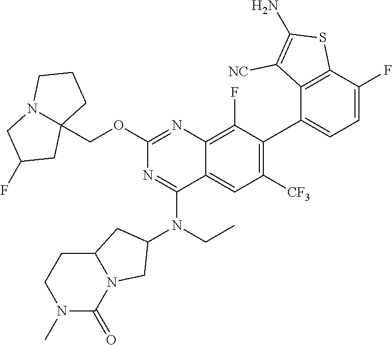 [SMILES: CCN(c1nc(OCC23CCCN2CC(F)C3)nc2c(F)c(-c3ccc(F)c4sc(N)c(C#N)c34)c(C(F)(F)F)cc12)C1CC2CCN(C)C(=O)N2C1] | |
| 822 | 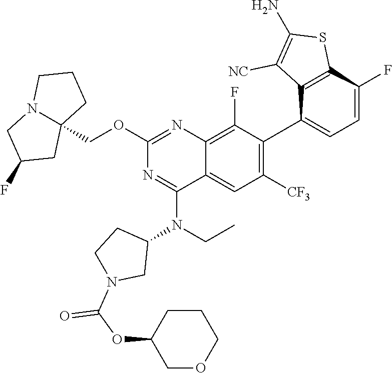 | LCMS m/z [M − 1]: 804.2 ¹H NMR (400 MHz, CD₃OD) δ 8.23 (s, 1H), 7.20-7.23 (m, 1H), 7.01 (t, 1H), 5.37 (s, 0.5H), 5.23 (s, 0.5H), 5.01-5.05 (m, 1H), 4.64-4.68 (m, 1H), 4.22-4.31 (m, 2H), 3.85-4.00 (m, 3H), 3.72-3.78 (m, 2H), 3.58-3.66 (m, 4H), 3.41-3.47 (m, 1H), 3.12-3.25 (m, 3H), 2.98- 3.02 (m, 1H), 2.44-2.49 (m, 1H), 2.20-2.37 (m, 3H), 2.11-2.17 (m, 1H), 1.79-2.01 (m, 6H), 1.57 (s, 1H), 1.45 (t, 3H). |
| 822a | 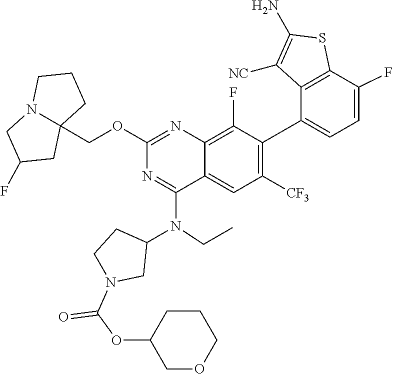 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2CCCOC2)C1] | |
| 823 | 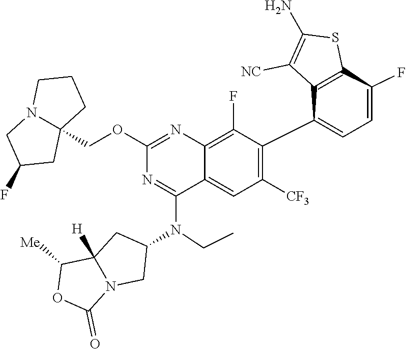 | LCMS m/z [M − 1]: 746.1 ¹H NMR (400 MHz, CD₃OD) δ 8.18 (s, 1H), 7.20-7.23 (m, 1H), 7.01 (t, 1H), 5.42 (s, 0.5H), 5.29 (s, 0.5H), 4.93-4.99 (m, 2H), 4.36 (d, 1H), 4.26 (d, 1H), 4.13-4.20 (m, 1H), 3.48-3.92 (m, 6H), 3.08-3.12 (m, 1H), 2.20- 2.63 (m, 5H), 2.01-2.04 (m, 3H), 1.93-1.98 (m, 1H), 1.46-1.52 (m, 6H). |
| 823a | 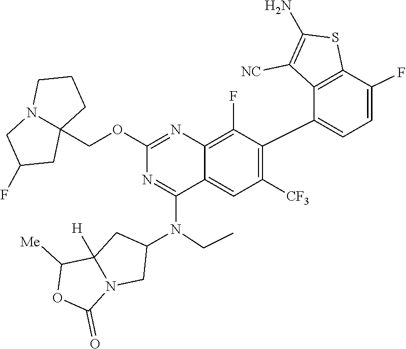 [SMILES: CCN(c1nc(OCC23CCCN2CC(F)C3)nc2c(F)c(-c3ccc(F)c4sc(N)c(C#N)c34)c(C(F)(F)F)cc12)C1CC2C(C)OC(=O)N2C1] | |
| 824 | 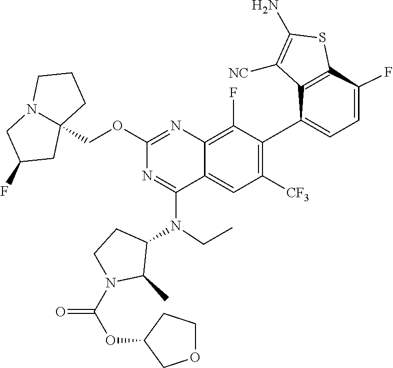 | LCMS m/z [M − 1]: 804.3 ¹H NMR (400 MHz, CD₃OD) δ 8.21 (s, 1H), 7.22 (t, 1H), 7.01 (t, 1H), 5.37 (s, 0.5H), 5.30-5.23 (m, 1.5H), 4.70-4.64 (m, 1H), 4.33-4.20 (m, 3H), 3.96-3.75 (m, 7H), 3.50-3.44 (m, 1H), 3.27-3.16 (m, 3H), 3.05-2.98 (m, 1H), 2.54-2.31 (m, 3H), 2.28-2.14 (m, 3H), 2.13- 1.95 (m, 4H), 1.93-1.85 (m, 1H), 1.42 (4, 3H), 1.36 (d, 3H). |
| 825 | 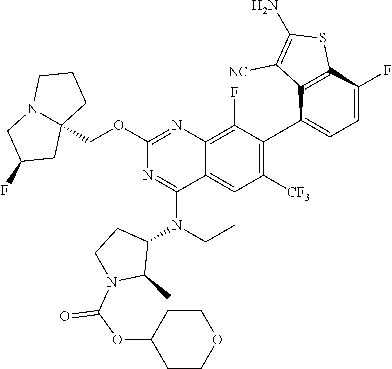 | LCMS m/z [M − 1]: 818.3 ¹H NMR (400 MHz, CD₃OD) δ 8.21 (s, 1H), 7.22 (t, 1H), 7.01 (t, 1H), 5.37 (s, 0.5H), 5.23 (s, 0.5H), 4.70- 4.65 (m, 1H), 4.28-4.21 (m, 3H), 3.95-3.77 (m, 5H), 3.61-3.45 (m, 3H), 3.27-3.15 (m, 3H), 3.05-2.97 (m, 1H), 2.54-2.32 (m, 3H), 2.29-2.2.07 (m, 3H), 2.03-1.85 (m, 5H), 1.73-1.63 (m, 2H), 1.46-1.33 (m, 6H). |
| 825a | 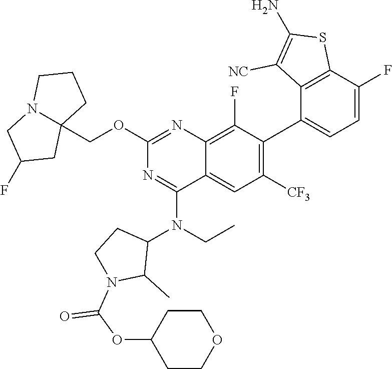 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2CCOCC2)C1C] | |
| 826 | 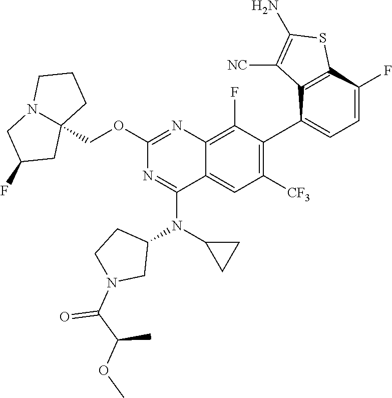 | LCMS m/z [M − 1]: 774.2 ¹H NMR (400 MHz, CD₃OD) δ 8.73-8.64 (m, 1H), 8.51 (brs, 0.34H), 7.27-7.18 (m, 1H), 7.01 (t, J = 9.0 Hz, 1H), 5.38 (s, 0.55H), 5.25 (s, 0.52H), 4.85-4.71 (m, 1H), 4.39-4.04 (m, 4H), 4.02-3.59 (m, 3H), 3.55-3.36 (m, 2H), 3.37-3.30 (m, 5H), 3.12-2.99 (m, 1H), 2.74-2.52 (m, 2H), 2.34-1.88 (m, 6H), 1.39-1.27 (m, 3H), 1.12- 0.93 (m, 2H), 0.88-0.64 (m, 2H). |
| 827 | 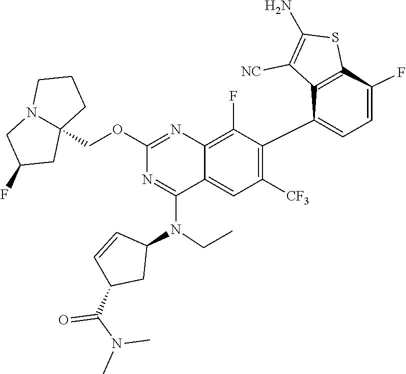 | LCMS m/z [M − 1]: 746.3 ¹H NMR (400 MHz, CD₃OD) δ = 8.26 (s, 1H), 7.22 (dd, J = 8.3, 5.0, 1H), 7.01 (t, J = 8.9, 1H), 5.46 (s, 0.5H), 5.32 (s, 0.5H), 5.02 (s, 1H), 4.43 (dd, J = 60.7, 11.2, 2H), 3.93- 3.75 (m, 2H), 3.47 (s, 3H), 3.15 (s, 4H), 2.95 (s, 3H), 2.60-1.67 (m, 13H), 1.46 (t, J = 6.9 Hz, 3H). |
| 827a | 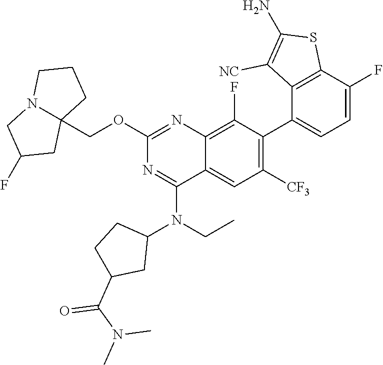 [SMILES: CCN(c1nc(OCC23CCCN2CC(F)C3)nc2c(F)c(-c3ccc(F)c4sc(N)c(C#N)c34)c(C(F)(F)F)cc12)C1CCC(C(=O)N(C)C)C1] | |
| 828 | 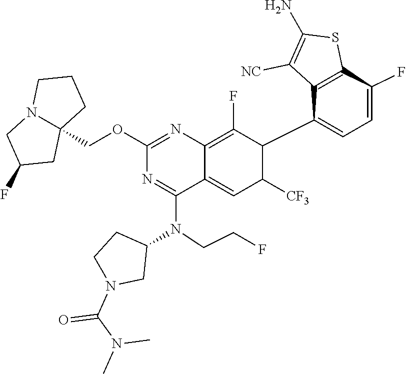 | LCMS m/z [M − 1]: 765.3 ¹H NMR (400 MHz, CD₃OD) δ = 8.20 (s, 1H), 7.22 (dd, J = 8.3, 5.0, 1H), 7.06-6.98 (m, 1H), 5.40 (s, 0.5H), 5.27 (s, 0.5H), 5.02-4.96 (m, 1H), 4.79 (d, J = 4.6, 0.5H), 4.66 (s, 0.5H), 4.32 (dd, J = 38.4, 10.6, 2H), 4.20- 3.99 (m, 2H), 3.83-3.76 (m, 1H), 3.74-3.61 (m, 2H), 3.53 (dd, J = 20.1, 12.0, 2H), 3.32 (s, 3H), 3.08 (s, 1H), 3.01-2.69 (m, 6H), 2.41-1.87 (m, 8H). |
| 828a | 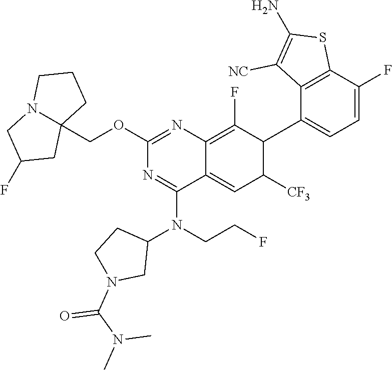 [SMILES: CN(C)C(=O)N1CCC(N(CCF)c2nc(OCC34CCCN3CC(F)C4)nc3c2=CC(C(F)(F)F)C(c2ccc(F)c4sc(N)c(C#N)c24)C=3F)C1] | |
| 829 | 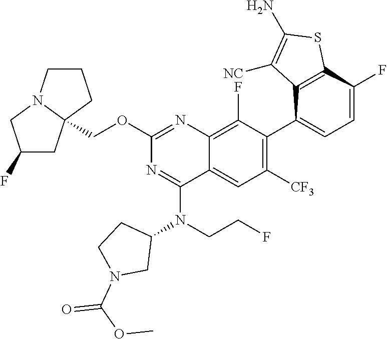 | LCMS m/z [M − 1]: 752.3 ¹H NMR (400 MHz, CD₃OD) δ = 8.20 (s, 1H), 7.26- 7.20 (m, 1H), 7.05-6.99 (m, 1H), 5.40 (s, 0.5H), 5.26 (s, 0.5H), 5.03-4.98 (m, 1H), 4.78 (d, J = 3.9, 0.5H), 4.66 (d, J = 3.5, 0.5H), 4.31 (dd, J = 32.2, 10.9, 2H), 4.10 (d, J = 25.6, 2H), 3.89 (dd, J = 11.3, 8.0, 1H), 3.79-3.58 (m, 5H), 3.50-3.37 (m, 2H), 3.29-3.19 (m, 3H), 3.08 (s, 1H), 2.41-1.92 (m, 8H). |
| 829a | 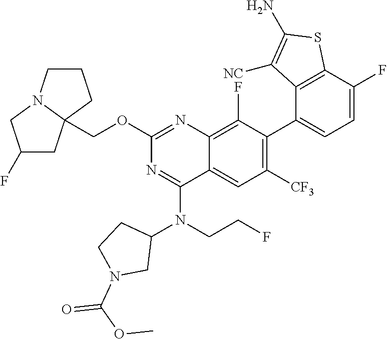 [SMILES: COC(=O)N1CCC(N(CCF)c2nc(OCC34CCCN3CC(F)C4)nc3c(F)c(-c4ccc(F)c5sc(N)c(C#N)c45)c(C(F)(F)F)cc23)C1] | |
| 830 | 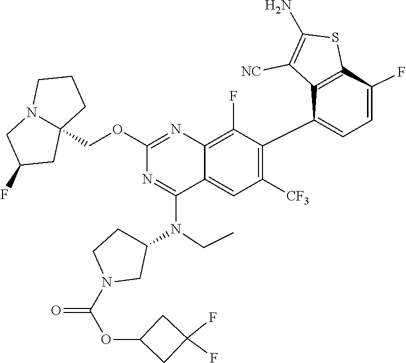 | LCMS m/z [M − 1]: 810.3 ¹H NMR (400 MHz, CD₃OD) δ 8.23 (s, 1H), 7.19-7.25 (m, 1H), 7.01 (t, 1H), 5.37 (s, 0.5H), 5.23 (s, 0.25H), 4.97-5.07 (m, 1H), 4.21-4.33 (m, 2H), 3.82-3.99 (m, 3H), 3.71-3.81 (m, 1H), 3.37-3.62 (m, 3H), 3.25 (s, 1H), 3.20 (s, 1H), 2.96-3.09 (m, 3H), 2.63-2.75 (m, 2H), 2.09-2.53 (m, 6H), 1.95-2.04 (m, 2H), 1.84-1.94 (m, 1H), 1.44 (t, 3H). |
| 830a | 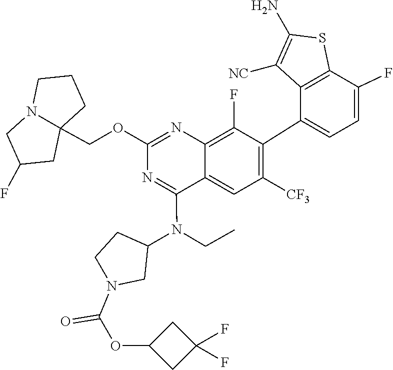 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2CC(F)(F)C2)C1] | |
| 831 | 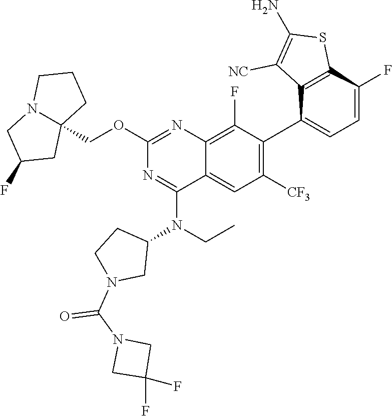 | LCMS m/z [M − 1]: 795.3 ¹H NMR (400 MHz, CD₃OD) δ 8.25 (s, 1H), 7.20-7.23 (m, 1H), 7.01 (t, 1H), 5.44 (s, 0.5H), 5.30 (s, 0.5H), 5.00 (t, 1H), 4.29-4.49 (m, 6H), 3.83-3.92 (m, 3H), 3.72 (t, 1H), 3.40-2.62 (m, 5H), 3.13 (m, 1H), 2.30-2.51 (m, 4H), 2.20 (t, 1H), 2.02-2.14 (m, 2H), 1.88-2.00 (m, 1H), 1.47 (t, 3H). |
| 831a | 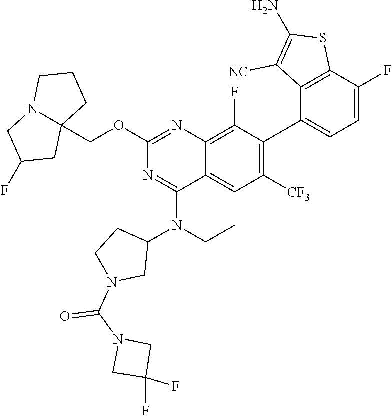 | |
| 832 | 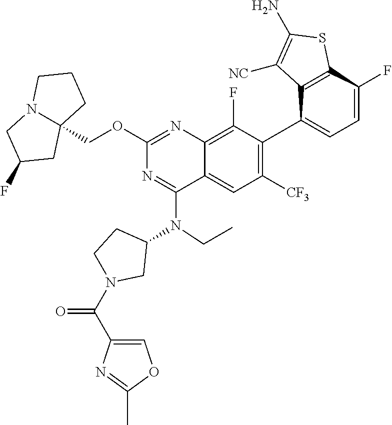 | LCMS m/z [M − 1]: 785.2 ¹H NMR (400 MHz, CD₃OD) δ 8.30 (s, 1H), 8.25 (s, 1H), 7.21 (t, 1H), 7.01 (t, 1H), 5.35 (s, 0.5H), 5.22 (s, 0.5H), 5.00-5.09 (m, 1H), 4.59 (s, 0.5H), 4.38-4.50 (m, 1H), 4.20-4.31 (m, 2H), 4.08-4.13 (m, 1H), 3.81-4.02 (m, 3.5H), 3.57-3.64 (m, 1H), 3.20-3.30 (m, 2H), 3.01- 3.03 (m, 1H), 2.54-2.59 (m, 1H), 2.45-2.49 (m, 3H), 2.22-2.40 (m, 3H), 2.07-2.11 (m, 1H), 1.93-1.99 (m, 2H), 1.85 (s, 1H), 1.46-1.52 (m, 3H). |
| 832a | 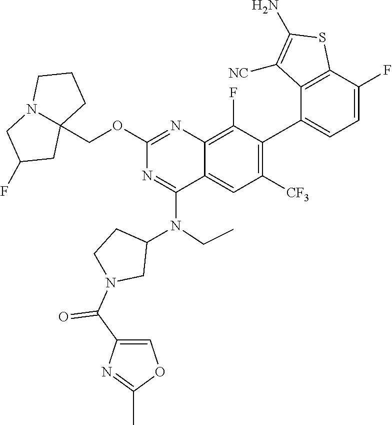 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2coc(C)n2)C1] | |
| 833 | 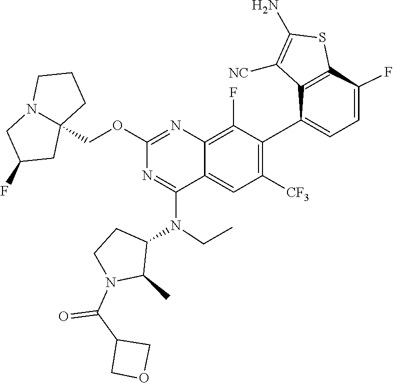 | LCMS m/z [M − 1]: 774.2 ¹H NMR (400 MHz, CD₃OD) δ 8.21 (s, 1H), 7.27-7.17 (m, 1H), 7.01 (t, 1H), 5.38 (s, 0.5H), 5.25 (s, 0.5H), 4.97-4.89 (m, 2H), 4.85-4.76 (m, 2H), 4.65-4.49 (m, 2H), 4.36-4.08 (m, 3H), 3.94-3.72 (m, 2H), 3.67-3.56 (m, 1H), 3.52-3.34 (m, 2H), 3.27-3.18 (m, 2H), 3.09- 2.99 (m, 1H), 2.57-2.46 (m, 1H), 2.45-2.27 (m, 2H), 2.26-2.18 (m, 1H), 2.17-2.09 (m, 1H), 2.06-1.83 (m, 3H), 1.46-1.27 (m, 6H). |
| 833a | 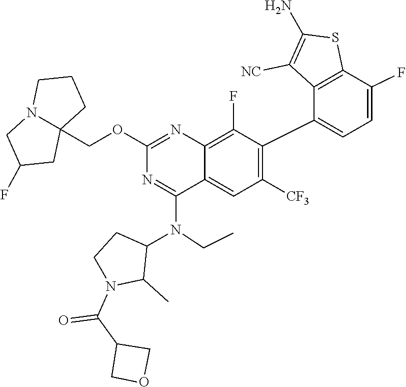 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2COC2)C1C] | |
| 834 | 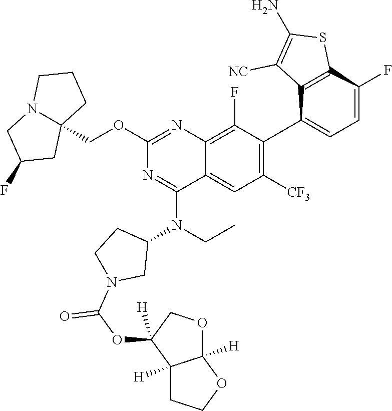 | LCMS m/z [M − 1]: 832.2 ¹H NMR (400 MHz, CD₃OD) δ 8.23 (s, 1H), 7.20-7.23 (m, 1H), 7.01 (t, 1H), 5.77-5.81 (m, 1H), 5.38 (s, 0.5H), 5.24 (s, 0.5H), 4.96-5.03 (m, 2H), 4.22-4.32 (m, 2H), 4.02-4.08 (m, 1H), 3.85-3.95 (m, 6H), 3.72-3.76 (m, 1H), 3.40-3.58 (m, 2H), 3.12-3.26 (m, 3H), 2.91-3.03 (m, 2H), 2.42-2.50 (m, 1H), 2.33-2.36 (m, 2H), 2.11- 2.24 (m, 3H), 1.89-2.02 (m, 4H), 1.44 (t, 3H). |
| 835 | 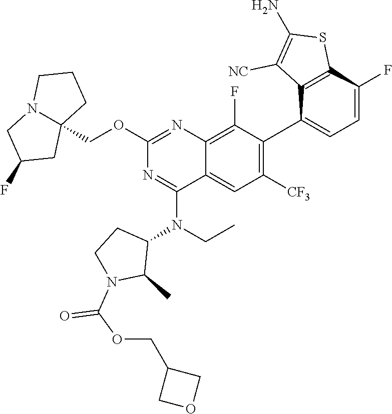 | LCMS m/z [M − 1]: 804.3 ¹H NMR (400 MHz, CD₃OD) δ 8.21 (s, 1H), 7.20-7.24 (m, 1H), 7.01 (t, 1H), 5.37 (s, 0.5H), 5.23 (s, 0.5H), 4.81 (s, 2H), 4.52-4.66 (m, 4H), 4.20-4.30 (m, 4H), 3.79-3.91 (m, 3H), 3.47-3.51 (m, 1H), 3.35-3.37 (m, 1H), 3.12-3.25 (m, 3H), 2.98-3.05 (m, 1H), 2.47-2.53 (m, 1H), 2.33-2.36 (m, 1H), 2.08-2.24 (m, 3H), 1.86- 2.01 (m, 3H), 1.36-1.44 (m, 6H). |
| 835a | 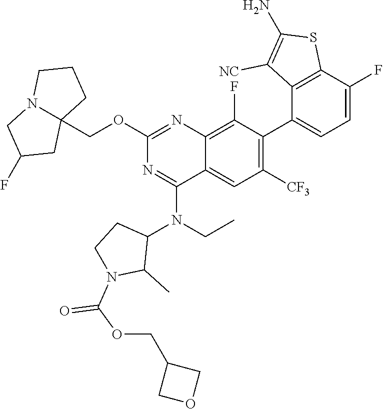 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OCC2COC2)C1C] | |
| 836 | 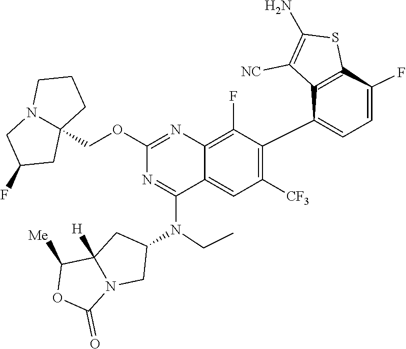 | LCMS m/z [M − 1]: 746.1 ¹H NMR (400 MHz, CD₃OD) δ 8.19 (s, 1H), 7.24-7.21 (m, 1H), 7.05-7.00 (m, 1H), 5.43 (s, 0.6H), 5.29 (s, 0.5H), 4.88 (m, 1H), 4.75-4.72 (m, 1H), 4.60 (s, 1H), 4.36-4.27 (m, 2H), 3.84-3.74 (m, 4H), 3.63-3.58 (m, 2H), 3.14 (m, 1H), 2.46-2.22 (m, 6H), 2.07-2.05 (m, 2H), 1.94 (s, 1H), 1.52-1.49 (m, 6H). |
| 837 | 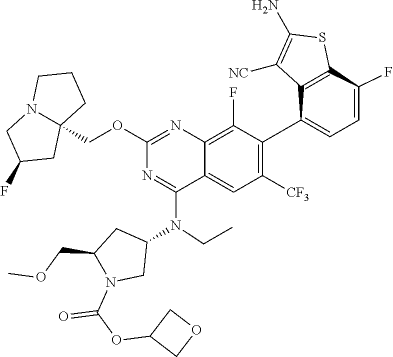 | LCMS m/z [M − 1]: 820.2 ¹H NMR (400 MHz, CD₃OD) δ 8.21 (s, 1H), 7.24-7.18 (m, 1H), 7.01 (t, 1H), 5.45-5.34 (m, 2.5H), 5.23 (s, 0.5H), 4.92-4.90 (m, 1H), 4.67-4.60 (m, 2H), 4.31-4.17 (m, 3H), 3.95-3.56 (m, 6H), 3.50-3.41 (m, 1H), 3.36- 3.32 (m, 3H), 3.24-3.18 (m, 2H), 3.04-2.97 (m, 1H), 2.69-2.60 (m, 1H), 2.35-1.84 (m, 8H), 1.45-1.37 (m, 3H). |
| 837a | 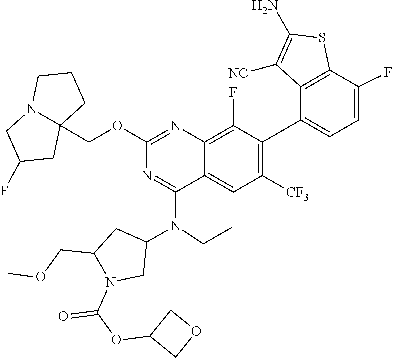 [SMILES: CCN(c1nc(OCC23CCCN2CC(F)C3)nc2c(F)c(-c3ccc(F)c4sc(N)c(C#N)c34)c(C(F)(F)F)cc12)C1CC(COC)N(C(=O)OC2COC2)C1] | |
| 838 | 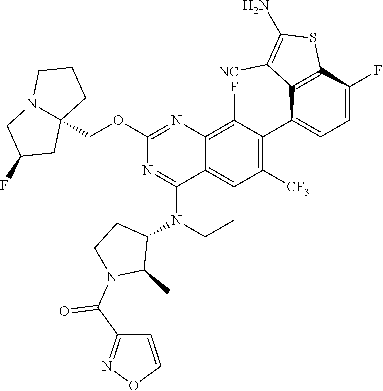 | LCMS m/z [M − 1]: 785.2 ¹H NMR (400 MHz, CD₃OD) δ 8.84-8.77 (m, 1H), 8.28-8.19 (m, 1H), 7.26-7.20 (m, 1H), 7.02 (t, 1H), 6.85-6.78 (m, 1H), 8.41 (s, 0.5H), 8.27 (s, 0.5H), 5.18- 5.07 (m, 1H), 5.82-4.74 (m, 1H), 4.69-4.57 (m, 2H), 4.36-4.27 (m, 2H), 3.96-3.84 (m, 2H), 3.268-3.57 (m, 1H), 3.44-3.35 (m, 2H), 3.14-3.09 (m, 1H), 2.69-2.61 (m, 1H), 2.55-2.36 (m, 2H), 2.32-2.25 (m, 1H), 2.16- 2.09 (m, 1H), 2.06-1.87 (m, 3H), 1.53-1.26 (m, 6H). |
| 838a | 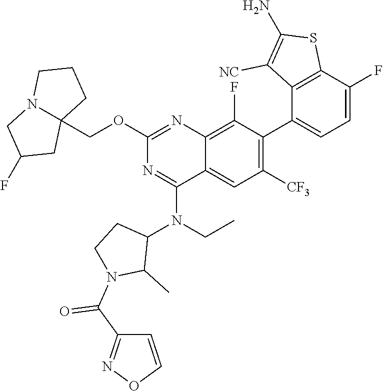 | |
| 839 | 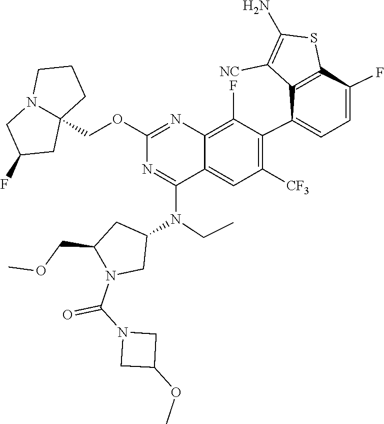 | LCMS m/z [M − 1]: 833.3 ¹H NMR (400 MHz, CD₃OD) δ = 8.21 (s, 1H), 7.21 (dd, J = 8.2, 5.0, 1H), 7.01 (t, J = 8.9, 1H), 5.38-5.23 (m, 2H), 4.37 (d, J = 4.0, 1H), 4.33-4.07 (m, 5H), 3.97-3.76 (m, 4H), 3.75-3.53 (m, 3H), 3.43 (dd, J = 9.4, 2.7, 1H), 3.34 (s, 3H), 3.28 (s, 3H), 3.23 (dd, J = 12.1, 9.1, 2H), 3.05- 2.98 (m, 1H), 2.58 (dt, J = 18.9, 9.4, 1H), 2.42-1.75 (m, 8H), 1.42 (t, J = 6.9, 3H). |
| 839a | 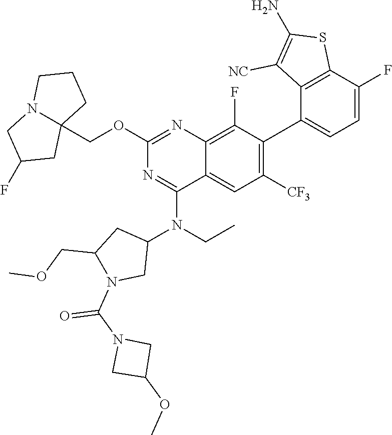 [SMILES: CCN(c1nc(OCC23CCCN2CC(F)C3)nc2c(F)c(-c3ccc(F)c4sc(N)c(C#N)c34)c(C(F)(F)F)cc12)C1CC(COC)N(C(=O)N2CC(OC)C2)C1] | |
| 840 | 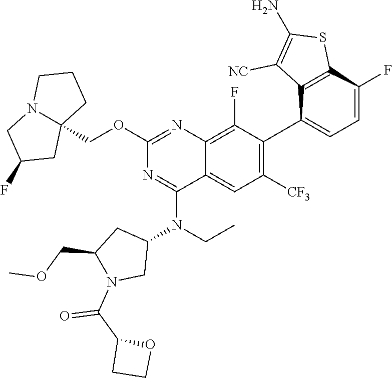 | LCMS m/z [M − 1]: 804.2 ¹H NMR (400 MHz, CD₃OD) δ = 8.20 (d, J = 5.5, 1H), 7.20 (dd, J = 8.3, 5.0, 1H), 7.01 (t, J = 8.9, 1H), 5.47- 5.20 (m, 3H), 4.75-4.57 (m, 2.5H), 4.50 (d, J = 8.7, 0.5H), 4.30-4.18 (m, 2H), 3.79 (ddt, J = 25.4, 20.2, 8.9, 5H), 3.49-3.43 (m, 1H), 3.34 (s, 3H), 3.22 (dd, J = 11.4, 8.4, 2H), 3.07-2.94 (m, 2H), 2.82-2.54 (m, 2H), 2.40- 1.80 (m, 8H), 1.42 (dd, J = 13.7, 6.8, 3H). |
| 840a | 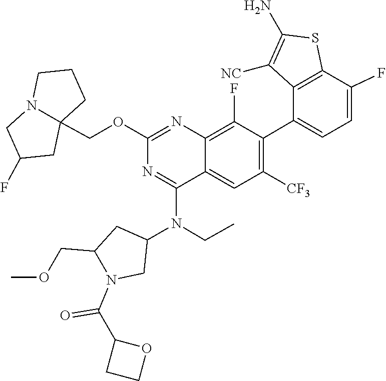 [SMILES: CCN(c1nc(OCC23CCCN2CC(F)C3)nc2c(F)c(-c3ccc(F)c4sc(N)c(C#N)c34)c(C(F)(F)F)cc12)C1CC(COC)N(C(=O)C2CCO2)C1] | |
| 841 | 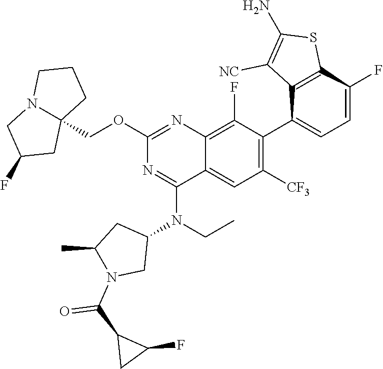 | LCMS m/z [M − 1]: 776.2 ¹H NMR (400 MHz, CD₃OD): δ 8.14-8.32 (m, 1 H), 7.16- 7.30 (m, 1 H), 6.97-7.05 (m, 1 H), 5.41 (s, 0.5 H), 5.22- 5.35 (m, 1.5 H), 4.88-5.01 (m, 1 H), 4.74-4.84 (m, 1 H), 4.51-4.69 (m, 1 H), 4.22-4.43 (m, 3 H), 3.77-3.95 (m, 3 H), 3.33-3.49 (m, 2 H), 3.02-3.14 (m, 1 H), 2.62-2.76 (m, 1 H), 1.92-2.39 (m, 8H), 1.56-1.83 (m, 2 H), 1.45-1.52 (m, 2 H), 1.31-1.44 (m, 3 H), 1.21-1.31 (m, 3 H). |
| 841a | 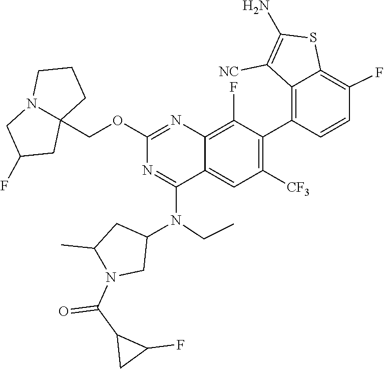 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)C2CC2F)C1] | |
| 842 | 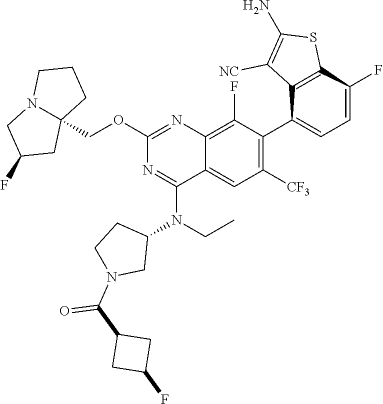 | LCMS m/z [M − 1]: 776.2 ¹H NMR (400 MHz, CD₃OD) δ 8.27-8.24 (m,, 1H), 7.23-7.20 (m, 1H), 7.01 (t, 1H), 5.41 (s, 0.5H), 5.28 (s, 0.5H), 5.09-4.96 (m, 1H), 4.61-4.56 (m, 0.5H), 4.40- 4.22 (m, 2.5H), 4.15-3.76 (m, 5H), 3.74-3.33 (m, 5H), 3.10 (s, 1H), 2.65-2.24 (m, 6H), 2.23-1.99 (m, 4H), 1.95-1.89 (m, 1H), 1.83-1.63 (m, 1H), 1.51-1.43 (m, 3H). |
| 842a | 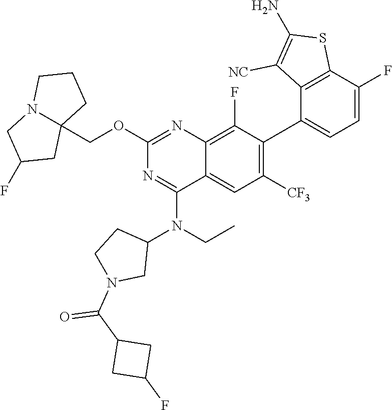 | |
| 843 | 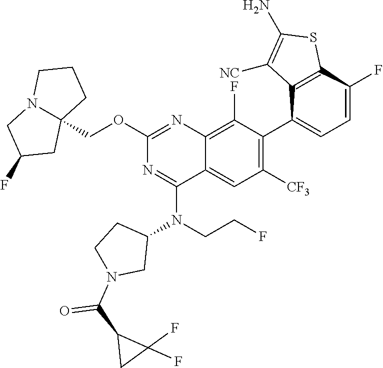 | LCMS m/z [M − 1]: 798.22 ¹H NMR (400 MHz, CD₃OD) δ 8.31-8.29 (m, 1H), 7.22 (s, 1H), 7.04-6.99 (m, 1H), 5.35 (s, 0.5H), 5.22 (s, 0.5H), 5.03 (m, 1H), 4.84-4.68 (m, 1H), 4.65 (s, 1H), 4.22-4.02 (m, 4H), 3.97-3.70 (m, 3H), 3.68-3.48 (m, 1H), 3.30-3.13 (m, 3H), 2.98-2.95 (m, 1H), 2.83-2.75 (m, 1H), 2.53-1.78 (m, 10H). |
| 843a | 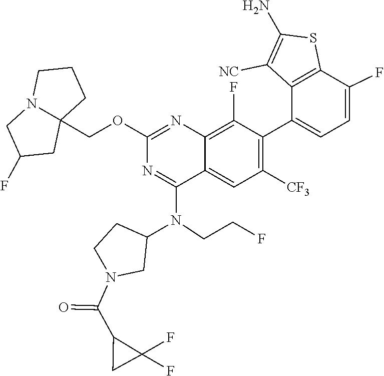 [SMILES: N#Cc1c(N)sc2c(F)ccc(-c3c(C(F)(F)F)cc4c(N(CCF)C5CCN(C(=O)C6CC6(F)F)C5)nc(OCC56CCCN5CC(F)C6)nc4c3F)c12] | |
| 844 | 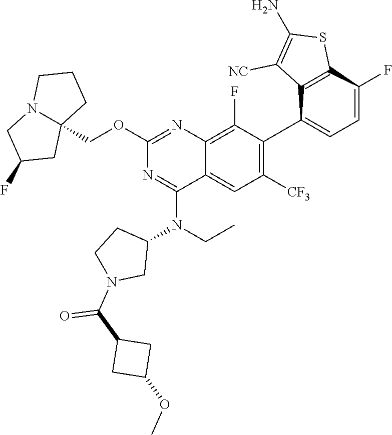 | LCMS m/z [M − 1]: 788.20 ¹H NMR (400 MHz, CD₃OD) δ 8.23 (s, 1H), 7.23-7.19 (m, 1H), 7.01 (t, 1H), 5.37 (s, 0.5H), 5.24 (s, 0.5H), 5.06-4.97 (m, 1H), 4.32-4.20 (m, 2H), 4.06-3.94 (m, 2H), 3.89-3.73 (m, 3H), 3.67-3.39 (m, 3H), 3.23 (d, 6H), 3.06-2.99 (m, 1H), 2.62-2.29 (m, 5H), 2.28-2.18 (m, 3H), 2.16-2.11 (m, 1H), 2.04-1.96 (m, 2H), 1.94- 1.86 (m, 1H), 1.50-1.40 (m, 3H). |
| 844a | 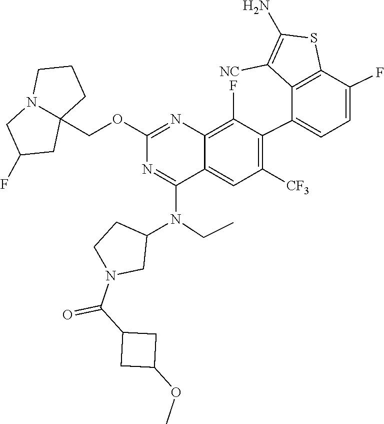 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CC(OC)C2)C1] | |
| 846 | 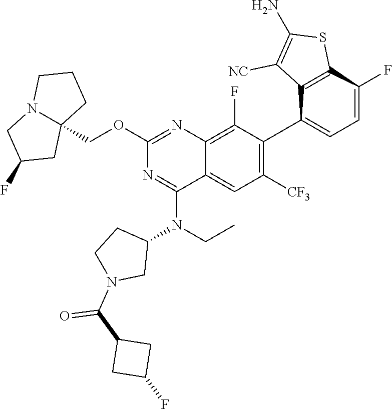 | LCMS m/z [M − 1]: 776.2 ¹H NMR (400 MHz, CD₃OD) δ 8.52 (s, 1H), δ 8.24 (s, 1H), 7.24-7.18 (m, 1H), 7.04-6.98 (t, 1H), 5.42-5.38 (m, 0.5H), 5.28-5.24 (m, 0.5H), 5.24-5.19 (m, 0.5H), 5.10- 5.06 (m, 0.5H), 5.05-4.97 (m, 1H), 4.37-4.22 (m, 2H), 4.04-3.95 (m, 1H), 3.93-3.75 (m, 3H), 3.65-3.32 (m, 5H), 3.28-3.26 (m, 1H), 3.11-3.03 (m, 1H), 2.68-2.23 (m, 8H), 2.19-2.11 (m, 1H), 2.08-1.98 (m, 2H), 1.95- 1.85 (m, 1H), 1.57-1.37 (m, 3H). |
| 847 | 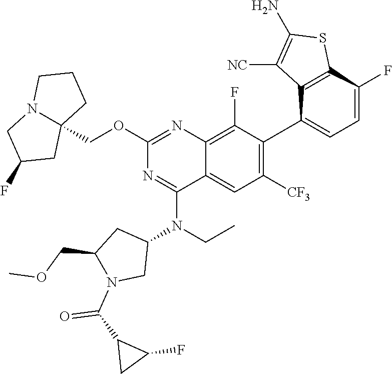 | LCMS m/z [M − 1]: 806.3 ¹H NMR (400 MHz, CD₃OD) δ 8.22 (d, J = 9.3 Hz, 1H), 7.24-7.18 (m, 1H), 7.01 (t, J = 8.9 Hz, 1H), 5.42 (dd, J = 17.2, 9.2 Hz, 1H), 5.36 (s, 0.5H), 5.22 (s, 0.5H), 4.95 (d, J = 6.4 Hz, 0.5H), 4.83-4.74 (m, 0.5H), 4.59 (s, 0.5H), 4.49 (s, 0.5H), 4.30-4.10 (m, 3H), 3.89 (ddd, J = 18.7, 14.7, 7.0 Hz, 2H), 3.80-3.65 (m, 1H), 3.62- 3.56 (m, 0.5H), 3.46 (dd, J = 9.4, 2.1 Hz, 0.5H), 3.36 (d, J = 5.9 Hz, 3H), 3.17 (dd, J = 22.7, 8.6 Hz, 3H), 2.99 (d, J = 5.6 Hz, 1H), 2.75 (ddd, J = 41.4, 21.8, 10.1 Hz, 1H), 2.42-2.16 (m, 4H), 2.15-1.81 (m, 6H), 1.43 (t, J = 6.9 Hz, 3H), 1.16-0.91 (m, 2H). |
| 847a | 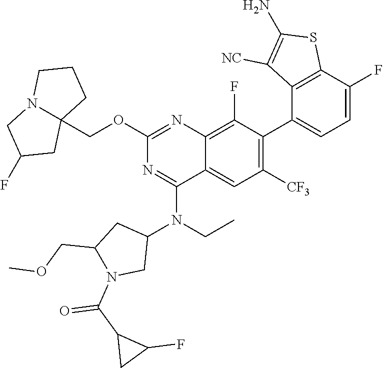 [SMILES: CCN(c1nc(OCC23CCCN2CC(F)C3)nc2c(F)c(-c3ccc(F)c4sc(N)c(C#N)c34)c(C(F)(F)F)cc12)C1CC(COC)N(C(=O)C2CC2F)C1] | |
| 848 | 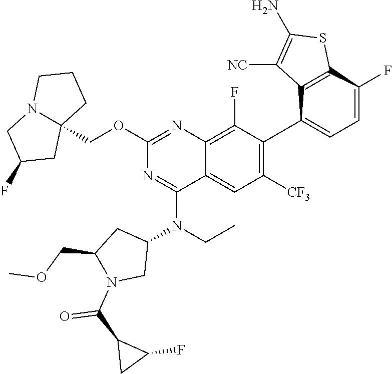 | LCMS m/z [M − 1]: 806.3 ¹H NMR (400 MHz, CD₃OD) δ 8.22 (d, 1H), 7.20-7.23 (m, 1H), 7.01 (t, 1H), 5.36-5.44 (m, 1.5H), 5.22 (s, 0.5H), 4.93 (s, 0.5H), 4.76 (s, 0.5H), 4.68 (s, 0.5H), 4.59 (s, 1H), 4.37 (s, 0.5H), 4.14-4.29 (m, 2H), 3.76-3.97 (m, 3H), 3.53-3.56 (m, 0.5H), 3.42-3.47 (m, 0.5H), 3.35 (d, 3H), 3.17-3.22 (m, 2H), 2.96-3.00 (m, 1H), 2.43-2.89 (m, 2H), 2.08-2.38 (m, 5H), 1.86-1.97 (m, 3H), 1.39- 1.49 (m, 3H), 0.87-0.94 (m, 2H). |
| 849 | 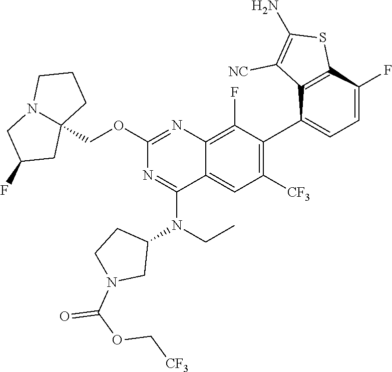 | LCMS m/z [M − 1]: 802.2 ¹H NMR (400 MHz, CD₃OD) δ 8.23 (s, 1H), 7.24-7.19 (m, 1H), 7.01 (t, 1H), 5.37 (s, 0.5H), 5.23 (s, 0.5H), 5.06-5.00 (m, 1H), 4.65-4.58 (m, 2H), 4.32-4.21 (m, 2H), 4.02-3.94 (m, 1H), 3.90-3.75 (m, 3H), 3.68-3.41 (m, 3H), 3.26-3.19 (m, 2H), 3.05-2.98 (m, 1H), 2.53- 2.45 (m, 1H), 2.39-2.33 (m, 1H), 2.28-2.08 (m, 3H), 2.02-1.86 (m, 3H), 1.45 (t, 3H). |
| 849a | 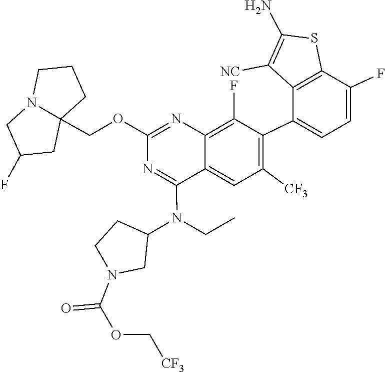 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OCC(F)(F)F)C1] | |
| 850 | 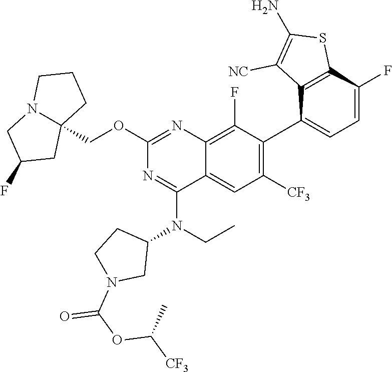 | LCMS m/z [M − 1]: 816.2 ¹H NMR (400 MHz, CD₃OD) δ 8.24 (d, 1H), 7.27-7.19 (m, 1H), 7.01 (t, 1H), 5.38 (s, 0.5H), 5.34-5.28 (m, 1H), 5.24 (s, 0.5H), 5.08-4.99 (m, 1H), 4.35-4.22 (m, 2H), 4.02-3.94 (m, 1H), 3.92-3.74 (m, 3H), 3.63-3.54 (m, 1H), 3.52-3.46 (m, 1H), 3.28-3.19 (m, 3H), 3.08-2.98 (m, 1H), 2.57-2.45 (m, 1H), 2.42-2.34 (m, 1H), 2.31- 2.07 (m, 3H), 2.03-1.87 (m, 3H), 1.48-1.39 (m, 6H). |
| 850a | 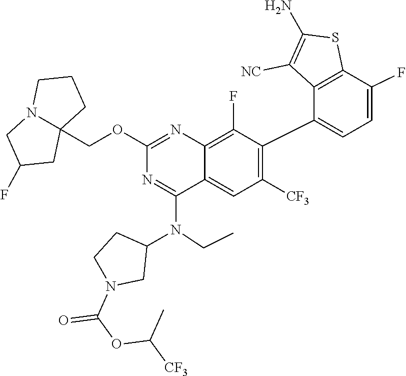 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC(C)C(F)(F)F)C1] | |
| 851 | 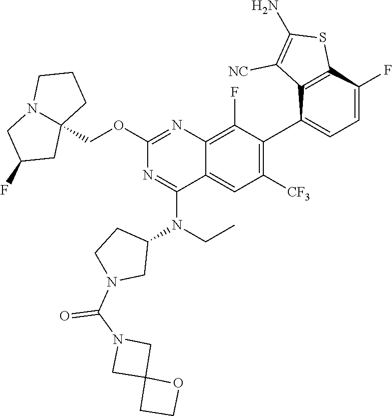 | LCMS m/z [M − 1]: 801.2 ¹H NMR (400 MHz, CD₃OD) δ 8.24 (s, 1H), 7.25-7.19 (m, 1H), 7.01 (t, 1H), 5.41 (s, 0.5H), 5.28 (s, 0.5H), 5.01-4.94 (m, 1H), 4.50 (t, 2H), 4.38-4.25 (m, 3H), 4.19 (s, 2H), 4.14-4.10 (m, 1H), 3.91-3.82 (m, 3H), 3.77-3.67 (m, 2H), 3.59-3.55 (m, 1H), 3.50-3.34 (m, 3H), 3.12- 3.05 (m, 1H), 2.87 (t, 2H), 2.48-2.26 (m, 4H), 2.20-2.14 (m, 1H), 2.07-2.00 (m, 2H), 1.96-1.89 (m, 1H), 1.48 (t, 3H). |
| 851a | 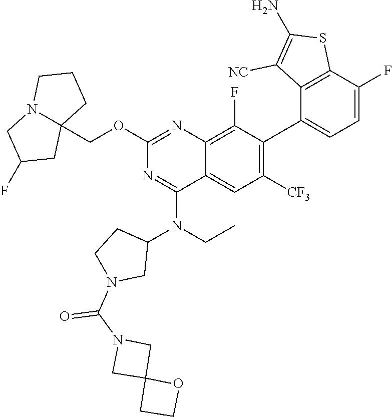 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N2CC3(CCO3)C2)C1] | |
| 852 | 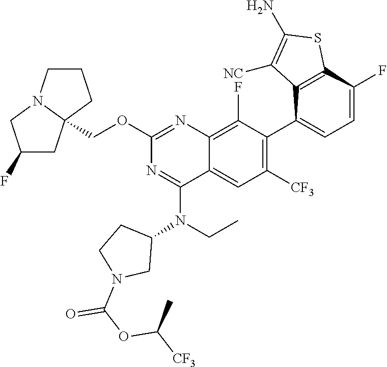 | LCMS m/z [M − 1]: 802.2 ¹H NMR (400 MHz, CD₃OD) δ 8.23 (s, 1H), 7.25-7.17 (m, 1H), 7.01 (t, 1H), 5.37 (s, 0.5H), 5.34-5.27 (m, 1H), 5.23 (s, 0.5H), 5.07-4.99 (m, 1H), 4.36-4.18 (m, 2H), 4.01-3.92 (m, 1H), 3.91-3.79 (m, 2H), 3.79-3.69 (m, 1H), 3.62-3.45 (m, 2H), 3.35-3.31 (m, 1H), 3.27-3.20 (m, 2H), 3.07-2.98 (m, 1H), 2.55-2.42 (m, 1H), 2.40- 2.33 (m, 1H), 2.33-2.08 (m, 3H), 2.03-1.86 (m, 3H), 1.53-1.33 (m, 6H). |
| 853 | 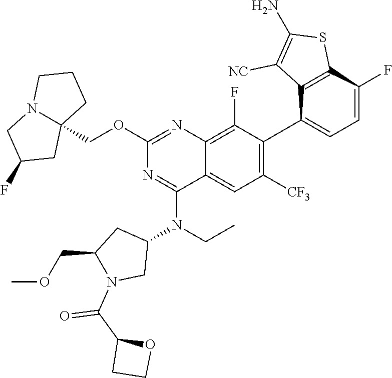 | LCMS m/z [M − 1]: 804.2 ¹H NMR (400 MHz, CD₃OD) δ 8.22 (s, 1H), 7.24-7.18 (m, 1H), 7.01 (t, 1H), 5.66-5.59 (m, 0.5H), 5.47-5.42 (m, 0.5H), 5.39-5.29 (m, 2H), 5.27-5.15 (m, 1H), 4.73- 4.66 (m, 1H), 4.55-4.48 (m, 1H), 4.43-4.28 (m, 2H), 4.01-3.86 (m, 2H), 3.81-3.67 (m, 2H), 3.52-3.37 (m, 4H), 3.34 (d, 3H), 3.18-3.11 (m, 1H), 2.94-2.81 (m, 2H), 2.65-2.54 (m, 1H), 2.38-2.16 (m, 4H), 2.13-1.91 (m, 4H), 1.48-1.38 (m, 3H). |
| 854 | 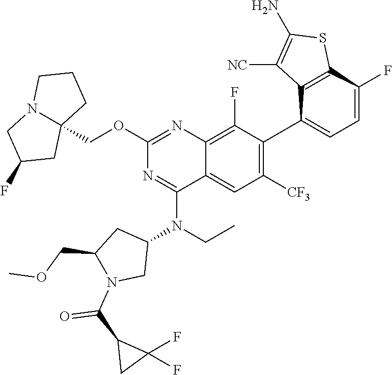 | LCMS m/z [M − 1]: 824.2 ¹H NMR (400 MHz, CD₃OD) δ 8.23 (d, 1H), 7.25-7.18 (m, 1H), 7.01 (t, 1H), 5.44-5.28 (m, 2H), 4.56-4.28 (m, 3H), 4.11-3.38 (m, 8H), 3.35 (d, 3H), 3.14-3.08 (m, 1H), 2.97-2.82 (m, 1H), 2.74-2.64 (m, 1H), 2.47-1.71 (m, 10H), 1.49-1.36 (m, 3H). |
| 854a | 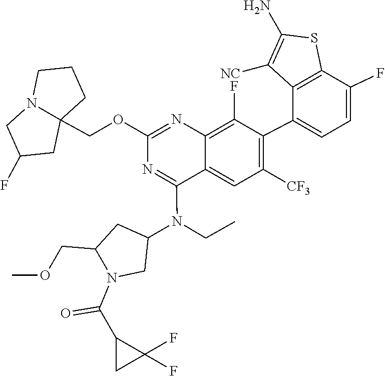 [SMILES: CCN(c1nc(OCC23CCCN2CC(F)C3)nc2c(F)c(-c3ccc(F)c4sc(N)c(C#N)c34)c(C(F)(F)F)cc12)C1CC(COC)N(C(=O)C2CC2(F)F)C1] | |
| 855 | 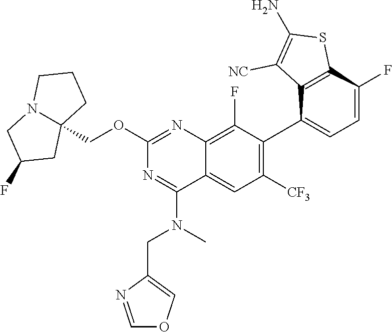 | LCMS m/z [M − 1]: 674.1 ¹H NMR (400 MHz, CD₃OD) δ 8.50 (s, 1H), 8.24 (s, 1H), 8.08 (s, 1H), 7.18-7.21 (m, 1H), 7.00 (t, 1H), 5.36 (s, 0.5H), 5.22 (s, 0.5H), 4.96 (s, 2H), 4.21-4.32 (m, 2H), 3.51 (s, 3H), 3.13-3.26 (m, 3H), 2.97-3.03 (m, 1H), 2.25-2.32 (m, 1H), 2.10-2.23 (m, 2H), 1.87-1.99 (m, 3H). |
| 855a | 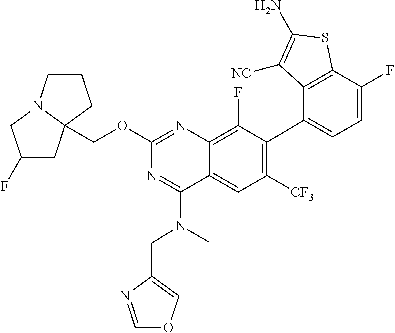 [SMILES: CN(Cc1cocn1)c1nc(OCC23CCCN2CC(F)C3)nc2c(F)c(-c3ccc(F)c4sc(N)c(C#N)c34)c(C(F)(F)F)cc12] | |
| 856 | 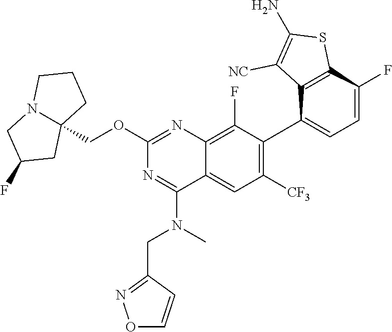 | LCMS m/z [M − 1]: 674.0 ¹H NMR (400 MHz, CD₃OD) δ 8.69 (d, 1H), 8.37 (s, 1H), 7.18-7.21 (m, 1H), 7.00 (t, 1H), 6.63 (d, 1H), 5.34 (s, 0.5H), 5.21 (s, 0.5H), 5.14 (d, 2H), 4.18-4.26 (m, 2H), 3.61 (s, 3H), 3.12-3.27 (m, 3H), 2.96-3.02 (m, 1H), 2.22-2.33 (m, 1H), 2.07-2.20 (m, 2H), 1.84-1.98 (m, 3H). |
| 856a | 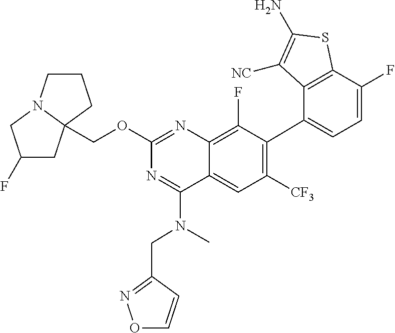 [SMILES: CN(Cc1ccon1)c1nc(OCC23CCCN2CC(F)C3)nc2c(F)c(-c3ccc(F)c4sc(N)c(C#N)c34)c(C(F)(F)F)cc12] | |
| 857 | 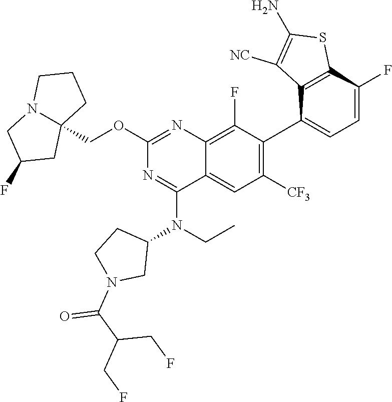 | LCMS m/z [M − 1]: 782.2 ¹H NMR (400 MHz, CD₃OD) δ 8.25-8.26 (m, 1H), 7.21 (t, 1H), 7.01 (t, 1H), 5.42 (s, 0.5H), 5.35 (s, 0.5H), 5.29 (s, 0.5H), 5.18 (s, 0.5H), 5.04-5.06 (m, 1H), 4.73-4.76 (m, 1H), 4.63-4.69 (m, 1H), 4.53-4.55 (m, 1H), 4.23- 4.38 (m, 3H), 4.03-4.05 (m, 1H), 3.56-3.90 (m, 6H), 3.46-3.47 (m, 1H), 3.37-3.40 (m, 1H), 3.12 (s, 1H), 2.05-2.56 (m, 8H), 1.43-1.51 (m, 3H). |
| 857a | 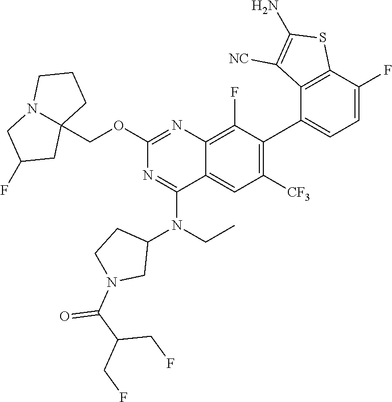 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C(CF)CF)C1] | |
| 858 | 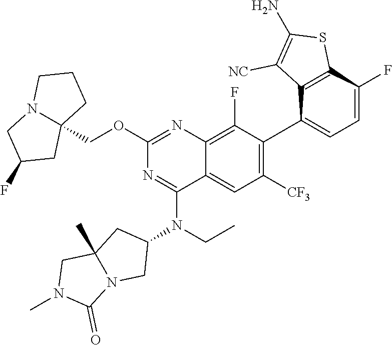 | LCMS m/z [M − 1]: 759.2 ¹H NMR (400 MHz, CD₃OD) δ 8.19 (s, 1H), 7.19-7.23 (m, 1H), 7.01 (t, 1H), 5.37 (m, 1.5H), 5.24 (s, 0.5H), 4.24-4.26 (m, 1H), 4.22-4.24 (m, 1H), 3.61-3.63 (m, 2H), 3.50-3.56 (m, 3H), 3.24-3.25 (m, 1H), 3.22-3.25 (m, 3H), 3.00-3.03 (m, 1H), 2.88 (s, 3H), 2.18-2.49 (m, 5H), 2.03-2.18 (m, 2H), 1.88-1.97 (m, 1H), 1.28-1.36 (m, 6H). |
| 858a | 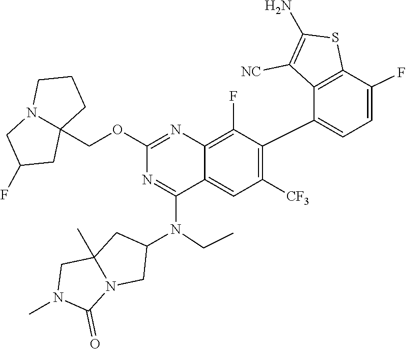 [SMILES: CCN(c1nc(OCC23CCCN2CC(F)C3)nc2c(F)c(-c3ccc(F)c4sc(N)c(C#N)c34)c(C(F)(F)F)cc12)C1CN2C(=O)N(C)CC2(C)C1] | |
| 859 | 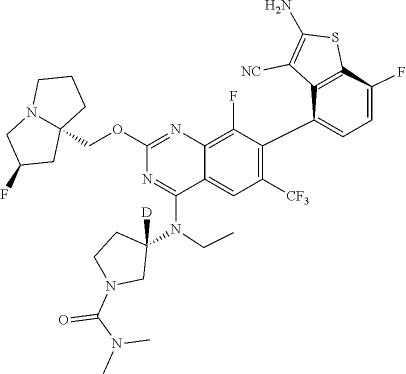 | LCMS m/z [M − 1]: 748.2 ¹H NMR (400 MHz, CD₃OD) δ 8.24 (s, 1H), 7.22-7.20 (m, 1H), 7.01 (t, J = 8.8 Hz, 1H), 5.37-5.24 (d, 1H), 4.32- 4.23 (m, 2H), 3.86-3.80 (m, 3H), 3.65-3.50 (m, 3H), 3.26-3.20 (m, 3H), 3.04-2.98 (m, 1H), 2.89 (s, 6H), 2.33-1.86 (m, 8H), 1.45 (t, J = 7.2 Hz, 3H). |
| 859a | 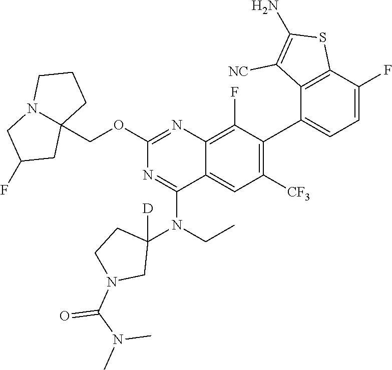 [SMILES: [2H]C1(N(CC)c2nc(OCC34CCCN3CC(F)C4)nc3c(F)c(-c4ccc(F)c5sc(N)c(C#N)c45)c(C(F)(F)F)cc23)CCN(C(=O)N(C)C)C1] | |
| 860 | 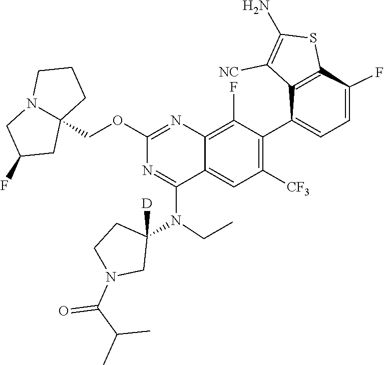 | LCMS m/z [M − 1]: 747.2 ¹H NMR (400 MHz, CD₃OD) δ 8.24 (d, J = 6.0, 1H), 7.23-7.20 (m, 1H), 7.01 (t, J = 8.8 Hz, 1H), 5.38-5.24 (d, J = 53.6 Hz, 1H), 4.31-4.22 (m, 2H), 4.16-3.81 (m, 4H), 3.71-3.57 (m, 2H), 3.44-3.39 (m, 1H), 3.30- 3.23 (m, 2H), 3.09-2.09 (m, 1H), 2.86-2.77 (m, 1H), 2.51-2.01 (m, 8H), 1.48-1.42 (m, 2H), 1.15-1.12 (m, 3H). |
| 860a | 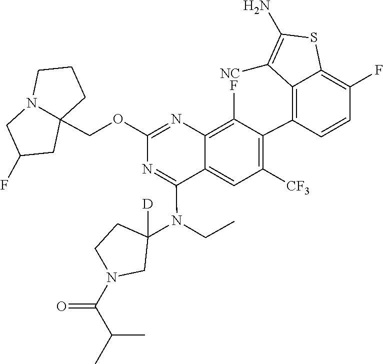 [SMILES: [2H]C1(N(CC)c2nc(OCC34CCCN3CC(F)C4)nc3c(F)c(-c4ccc(F)c5sc(N)c(C#N)c45)c(C(F)(F)F)cc23)CCN(C(=O)C(C)C)C1] | |
| 861 | 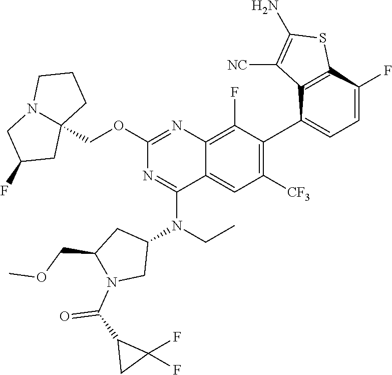 | LCMS m/z [M − 1]: 824.26 ¹H NMR (400 MHz, CD₃OD) δ 8.25 (s, 1H), 7.24- 7.22 (m, 1H), 7.04-6.99 (m, 1H), 5.49 (s, 0.5H), 5.37 (s, 1.5H), 4.59-4.41 (m, 2.5H), 4.40-4.38 (m, 0.5H), 4.16- 3.45 (m, 9H), 3.45-3.30 (m, 3H), 3.30-3.28 (m, 1H), 3.02-2.99 (m, 0.5H), 2.76-2.73 (m, 1.5H), 2.41-1.79 (m, 9H), 1.47-1.44 (m, 3H). |
| 862 | 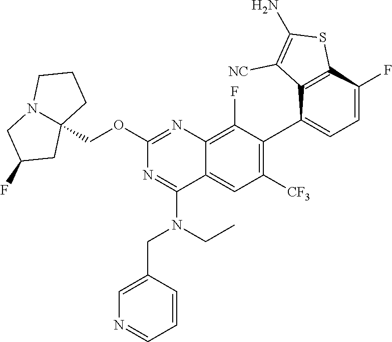 | LCMS m/z [M − 1]: 698.1 ¹H NMR (400 MHz, CD₃OD): 8.65 (s, 1 H), 8.50-8.51 (m, 1 H), 8.17 (s, 1 H), 7.92 (d, J = 11.6 Hz, 1 H), 7.43- 7.50 (m, 1 H), 7.16-7.23 (m, 1 H), 6.96-7.04 (m, 1 H), 5.44 (s, 0.5 H), 5.31 (s, 0.5 H), 5.11-5.19 (m, 2 H), 4.21-4.40 (m, 2 H), 3.91-4.04 (m, 2 H), 3.39-3.64 (m, 3 H), 3.12-3.20 (m, 1 H), 1.90-2.41 (m, 6 H), 1.51-1.62 (m, 3 H). |
| 862a | 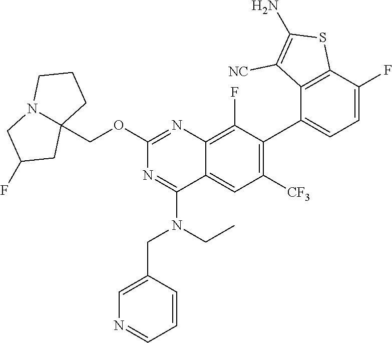 [SMILES: CCN(Cc1cccnc1)c1nc(OCC23CCCN2CC(F)C3)nc2c(F)c(-c3ccc(F)c4sc(N)c(C#N)c34)c(C(F)(F)F)cc12] | |
| 863 | 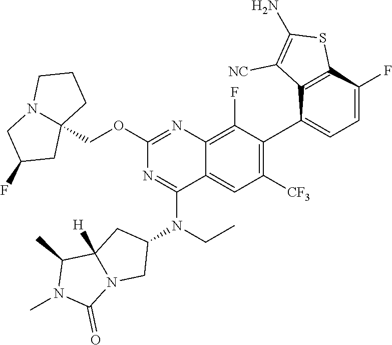 | LCMS m/z [M − 1]: 759.2 ¹H NMR (400 MHz, CD₃OD) δ 8.20 (s, 1H), 7.19-7.23 (m, 1H), 6.99-7.03 (t, 1H), 5.38 (s, 0.5H), 5.25 (s, 0.5H), 5.16-5.23 (m, 1H), 4.34 (d, 1H), 4.25 (d, 1H), 3.58-3.82 (m, 4H), 3.45-3.57 (m, 2H), 3.25 (s, 3H), 2.99-3.08 (m, 1H), 2.84 (s, 2H), 1.90-2.38 (m, 9H), 1.12-1.41 (m, 6H). |
| 863a | 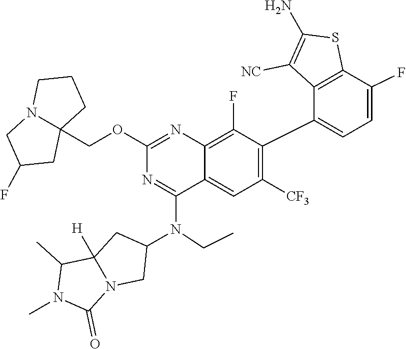 [SMILES: CCN(c1nc(OCC23CCCN2CC(F)C3)nc2c(F)c(-c3ccc(F)c4sc(N)c(C#N)c34)c(C(F)(F)F)cc12)C1CC2C(C)N(C)C(=O)N2C1] | |
| 864 | 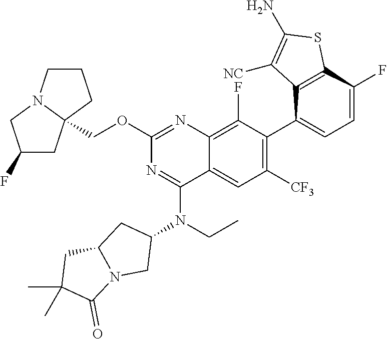 | LCMS m/z [M − 1]: 758.3 ¹H NMR (400 MHz, CD₃OD) δ 8.22 (s, 1H), 7.25- 7.18 (m, 1H), 7.01 (t, J = 8.9 Hz, 1H), 5.40-5.19 (m, 2H), 4.36-4.17 (m, 2H), 4.11-3.97 (m, 1H), 3.89- 3.73 (m, 2H), 3.72-3.56 (m, 2H), 3.28-3.17 (m, 3H), 3.09-2.95 (m, 1H), 2.49-2.41 (m, 1H), 2.38-2.20 (m, 3H), 2.20-2.12 (m, 1H), 2.07-1.94 (m, 3H), 1.93- 1.81 (m, 2H), 1.43 (t, J = 6.9 Hz, 3H), 1.25 (d, J = 12.9 Hz, 6H). |
| 864a | 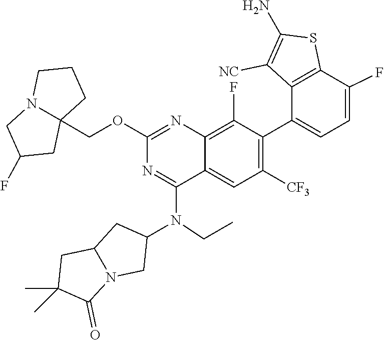 [SMILES: CCN(c1nc(OCC23CCCN2CC(F)C3)nc2c(F)c(-c3ccc(F)c4sc(N)c(C#N)c34)c(C(F)(F)F)cc12)C1CC2CC(C)(C)C(=O)N2C1] | |
| 865 | 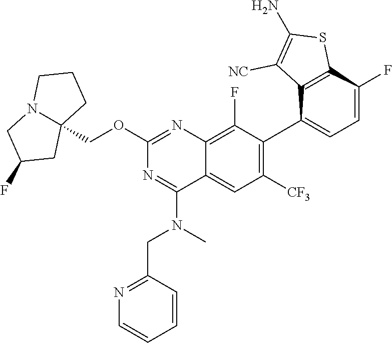 | LCMS m/z [M − 1]: 684.1 ¹H NMR (400 MHz, CD₃OD) δ 8.57 (d, 1H), 8.36 (s, 1H), 7.87 (t, 1H), 7.53 (d, 1H), 7.38 (t, 1H), 7.18 (t, 1H), 6.99 (t, 1H), 5.33 (s, 0.5H), 5.19 (s, 0.5H), 5.16 (s, 2H), 4.17 (d, 1H), 4.09 (d, 1H), 3.62 (s, 3H), 3.17-3.27 (m, 3H), 2.95-3.01 (m, 1H), 2.14-2.30 (m, 2H), 2.04- 2.06 (m, 1H), 1.90-1.96 (m, 2H), 1.78-1.87 (m, 1H). |
| 865a | 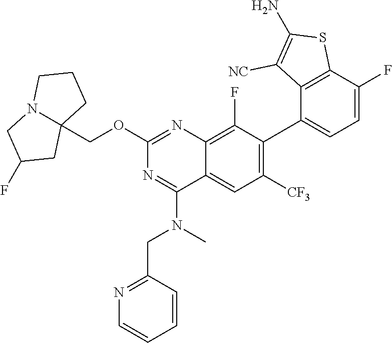 [SMILES: CN(Cc1ccccn1)c1nc(OCC23CCCN2CC(F)C3)nc2c(F)c(-c3ccc(F)c4sc(N)c(C#N)c34)c(C(F)(F)F)cc12] | |
| 866 | 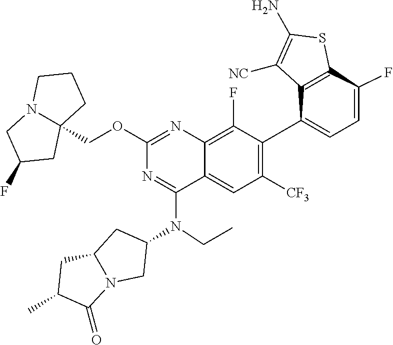 | LCMS m/z [M − 1]: 744.3 ¹H NMR (400 MHz, CD₃OD) δ 8.20 (s, 1H), 7.27- 7.15 (m, 1H), 7.08-6.92 (m, 1H), 5.37 (s, 0.5H), 5.24 (s, 0.5H), 5.19-5.06 (m, 1H), 4.34-4.04 (m, 3H), 3.94- 3.69 (m, 3H), 3.63-3.43 (m, 1H), 3.28-3.16 (m, 3H), 3.09-2.94 (m, 1H), 2.85-2.69 (m, 1H), 2.43- 2.31 (m, 2H), 2.29-1.83 (m, 8H), 1.47 (t, J = 7.0 Hz, 3H), 1.30 (d, J = 7.4 Hz, 3H). |
| 866a | 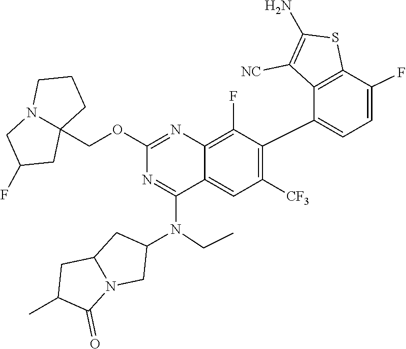 [SMILES: CCN(c1nc(OCC23CCCN2CC(F)C3)nc2c(F)c(-c3ccc(F)c4sc(N)c(C#N)c34)c(C(F)(F)F)cc12)C1CC2CC(C)C(=O)N2C1] | |
| 867 | 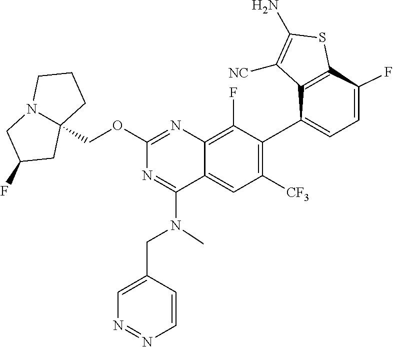 | LCMS m/z [M − 1]: 684.0 ¹H NMR (400 MHz, CD₃OD) δ 8.55 (d, J = 6.1 Hz, 2H), 8.28 (s, 1H), 7.49 (d, J = 6.0 Hz, 2H), 7.23-7.14 (m, 1H), 7.03-6.96 (m, 1H), 5.48 (s, 0.5H), 5.15- 5.31 (m, 2H), 5.11 (s, 0.5H), 4.11 (dd, J = 32.7, 10.6 Hz, 2H), 3.66-3.43 (m, 3H), 3.16 (d, J = 23.9 Hz, 2H), 2.02 (ddd, J = 91.8, 46.8, 27.6 Hz, 8H). |
| 867a | 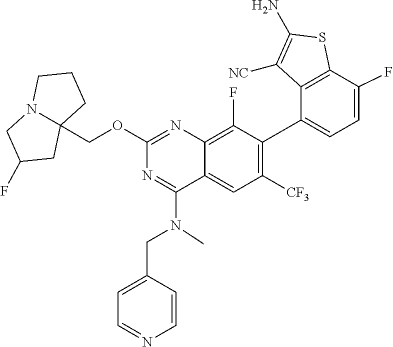 [SMILES: CN(Cc1ccncc1)c1nc(OCC23CCCN2CC(F)C3)nc2c(F)c(-c3ccc(F)c4sc(N)c(C#N)c34)c(C(F)(F)F)cc12] | |
| 868 | 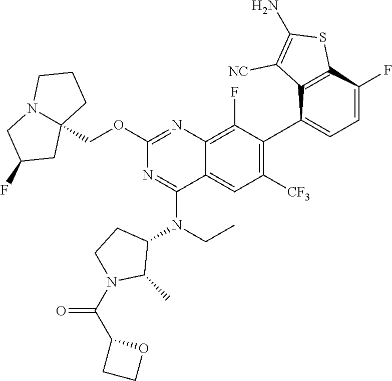 | LCMS m/z [M − 1]: 774.30 ¹H NMR (400 MHz, CD₃OD) δ 8.38 (s, 1H), 7.25-7.22 (m, 1H), 7.03 (t, 1H), 5.63 (s, 0.5H), 5.51 (s, 0.5H), 5.35-5.32 (m, 0.5H), 5.13-5.10 (m, 0.5H), 4.82-4.70 (m, 3H), 4.65-4.59 (m, 1H), 4.14-3.95 (m, 3H), 3.94-3.88 (m, 2H), 3.82-3.70 (m, 2H), 3.49-3.43 (m, 1H), 2.78- 2.45 (m, 5H), 2.42-2.26 (m, 3H), 2.22-1.99 (m, 4H), 1.56-1.42 (m, 3H), 1.10-0.93 (m, 3H). |
| 869 | 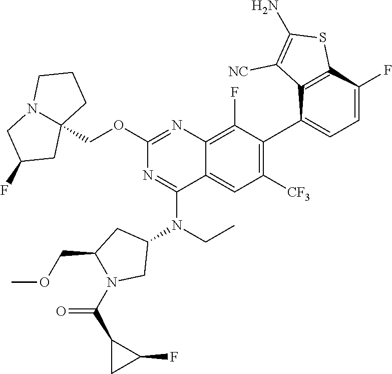 | LCMS m/z [M − 1]: 806.3 ¹H NMR (400 MHz, CD₃OD) δ 8.24 (d, 1H), 7.20-7.26 (m, 1H), 7.02 (t, 1H), 5.39-5.46 (m, 0.5H), 5.28 (s, 0.5H), 5.26 (s, 0.5H), 4.94-4.96 (m, 0.5H), 4.76-4.81 (m, 0.5H), 4.62-4.68 (m, 0.5H), 4.59 (s, 1H), 4.41-4.45 (m, 0.5H), 4.26-4.33 (m, 1H), 4.14 (t, 1H), 3.93-4.00 (m, 1H), 3.78-3.91 (m, 2H), 3.63-3.72 (m, 1H), 3.54- 3.62 (m, 1H), 3.47-3.51 (m, 1H), 3.38 (s, 3H), 3.22-3.29 (m, 2H), 3.00-3.08 (m, 1H), 2.63-2.75 (m, 1H), 2.29- 2.43 (m, 2H), 2.22-2.28 (m, 1H), 2.07-2.20 (m, 2H), 1.90-2.06 (m, 3H), 1.37-1.51 (m, 3H), 1.03-1.18 (m, 2H). |
| 870 | 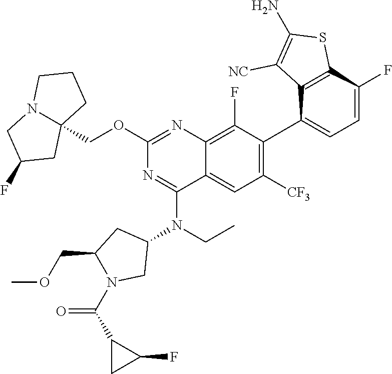 | LCMS m/z [M − 1]: 806.3 ¹H NMR (400 MHz, CD₃OD) δ 8.22 (d, 1H), 7.19-7.25 (m, 1H), 7.01 (t, 1H), 5.36-5.42 (m, 1.5H), 5.24 (s, 0.5H), 4.80-4.85 (m, 0.5H), 4.58-4.74 (m, 1.5H), 4.39- 4.44 (m, 0.5H), 4.22-4.34 (m, 2H), 4.12-4.18 (m, 0.5H), 3.91-3.99 (m, 1H), 3.69-3.89 (m, 2.5H), 3.58-3.66 (m, 1H), 3.54-3.58 (m, 0.5H), 3.32-3.45 (m, 3H), 3.21-3.27 (m, 2H), 2.98-3.07 (m, 1H), 2.63-2.79 (m, 1H), 1.85- 2.51 (m, 9H), 1.38-1.47 (m, 3H), 1.23-1.33 (m, 2H). |
| 871 | 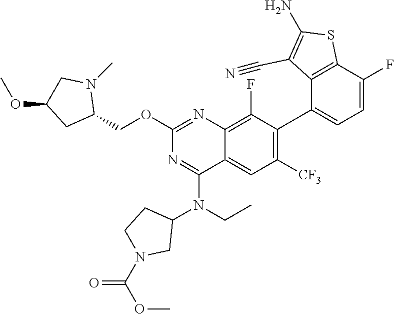 [SMILES: CCN(c1nc(OC[C@@H]2C[C@@H](OC)CN2C)nc2c(F)c(-c3ccc(F)c4sc(N)c(C#N)c34)c(C(F)(F)F)cc12)C1CCN(C(=O)OC)C1] | LCMS m/z [M − 1]: 720.3 ¹H NMR (400 MHz, CD₃OD) δ 8.28 (s, 1H), 7.22 (dd, J = 5.4, 2.3 Hz, 1H), 7.03 (t, J = 8.7 Hz, 1H), 5.07 (q, J = 8.2 Hz, 1H), 5.01-4.89 (m, 1H), 4.66 (q, J = 5.5 Hz, 1H), 4.15 (d, J = 28.1 Hz, 2H), 3.99-3.75 (m, 4H), 3.72 (s, 3H), 3.57 (t, J = 8.3 Hz, 1H), 3.50-3.41 (m, 2H), 3.39 (s, 3H), 3.13 (s, 3H), 2.61-2.31 (m, 3H), 2.28-2.08 (m, 1H), 1.47 (t, J = 7.0 Hz, 3H). |
| 871a | 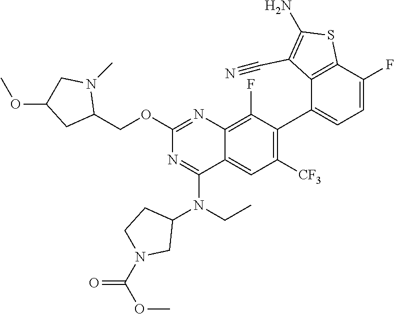 [SMILES: CCN(c1nc(OCC2CC(OC)CN2C)nc2c(F)c(-c3ccc(F)c4sc(N)c(C#N)c34)c(C(F)(F)F)cc12)C1CCN(C(=O)OC)C1] | |
| 872 | 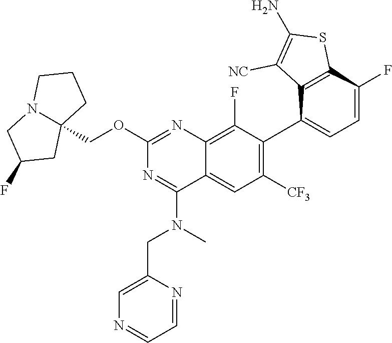 | LCMS m/z [M − 1]: 685.1 ¹H NMR (400 MHz, CD₃OD) δ 8.78 (s, 1H), 8.61 (s, 1H), 8.54 (d, 1H), 8.46 (s, 1H), 7.18-7.22 (m, 1H), 7.00 (t, 1H), 5.31 (s, 0.5H), 5.20 (s, 2H), 5.18 (s, 0.5H), 4.09 (d, 1H), 4.02 (d, 1H), 3.72 (s, 3H), 3.13-3.22 (m, 3H), 2.93-2.99 (m, 1H), 2.12-2.22 (m, 2H), 2.01-2.03 (m, 1H), 1.88-1.95 (m, 2H), 1.79-1.85 (m, 1H). |
| 872a | 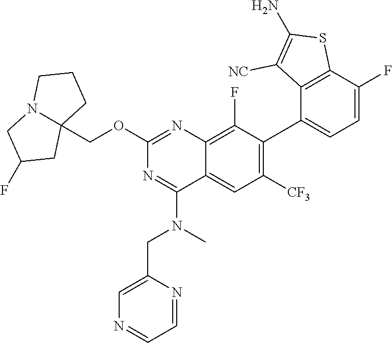 [SMILES: CN(Cc1cnccn1)c1nc(OCC23CCCN2CC(F)C3)nc2c(F)c(-c3ccc(F)c4sc(N)c(C#N)c34)c(C(F)(F)F)cc12] | |
| 873 | 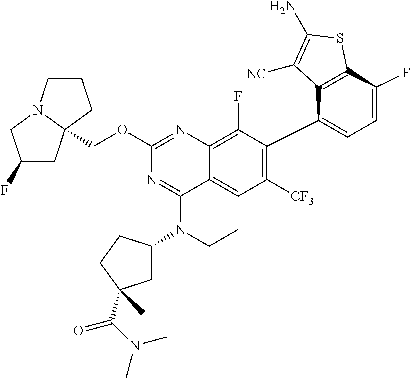 | LCMS m/z [M − 1]: 760.3 ¹H NMR (400 MHz, CD₃OD) δ 8.51 (brs, 0.48H), 8.22 (s, 1H), 7.26-7.18 (m, 1H), 7.01 (t, J = 8.8 Hz, 1H), 5.43 (s, 0.51H), 5.30 (s, 0.52H), 4.99-4.89 (m, 1H), 4.49- 4.28 (m, 2H), 3.90-3.66 (m, 2H), 3.51-3.34 (m, 3H), 3.22-2.96 (m, 7H), 2.61-1.88 (m, 11H), 1.79-1.69 (m, 1H), 1.49-1.29 (m, 6H). |
| 873a | 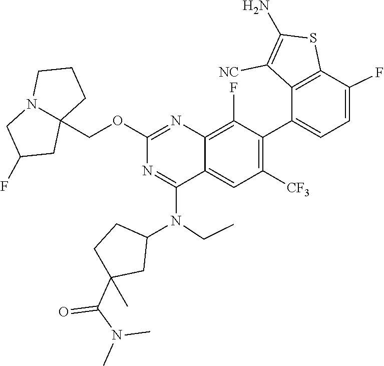 [SMILES: CCN(c1nc(OCC23CCCN2CC(F)C3)nc2c(F)c(-c3ccc(F)c4sc(N)c(C#N)c34)c(C(F)(F)F)cc12)C1CCC(C)(C(=O)N(C)C)C1] | |
| 874 | 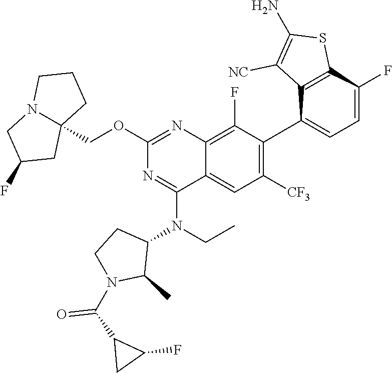 | LCMS m/z [M − 1]: 776.4 ¹H NMR (400 MHz, CD₃OD) δ 8.21 (s, 1H), 7.27-7.17 (m, 1H), 7.05-6.96 (m, 1H), 5.41 (s, 0.5H), 5.24 (s, 0.5H), 4.99-4.87 (m, 1H), 4.83-4.71 (m, 1H), 4.70-4.43 (m, 2H), 4.37-4.07 (m, 3H), 3.87-3.74 (m, 2H), 3.55- 3.33 (m, 1H), 3.28-3.19 (m, 2H), 3.08-2.98 (m, 1H), 2.62-2.39 (m, 2H), 2.37-2.15 (m, 3H), 2.13-1.86 (m, 4H), 1.73-1.58 (m, 1H), 1.57-1.40 (m, 3H), 1.38-1.31 (m, 3H), 1.13-1.05 (m, 1H). |
| 874a | 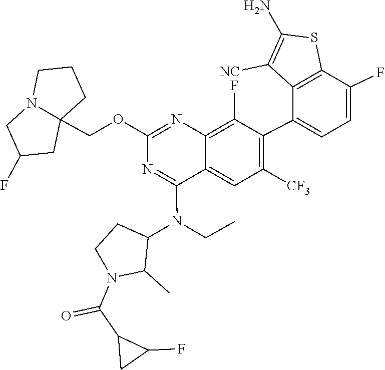 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CC2F)C1C] | |
| 875 | 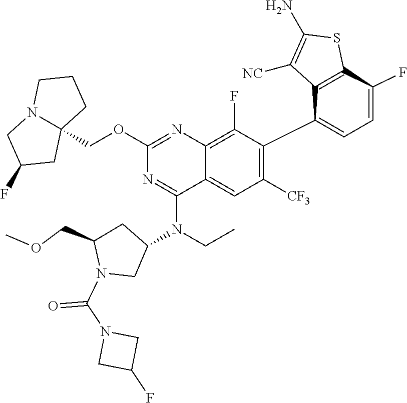 | LCMS m/z [M − 1]: 821.2 ¹H NMR (400 MHz, CD₃OD) δ 8.20 (s, 1H), 7.19-7.22 (m, 1H), 7.01 (t, 1H), 5.30-5.36 (m, 2H), 5.23 (s, 1H), 43.6-4.44 (m, 2H), 4.23-4.28 (d, 1H), 4.18-4.22 (d, 1H), 3.98-4.12 (m, 2H), 3.87-3.92 (m, 1H), 3.79 (t, 1H), 3.68-3.72 (m, 1H), 3.55-3.58 (m, 2H), 3.42-3.44 (m, 1H), 3.34 (s, 3H), 3.13-3.21 (m, 2H), 2.96-2.99 (m, 1H), 2.55-2.63 (m, 1H), 2.09-2.31 (m, 5H), 1.93-2.03 (m, 3H), 1.87-1.88 (m, 1H), 1.31 (t, 3H). |
| 875a | 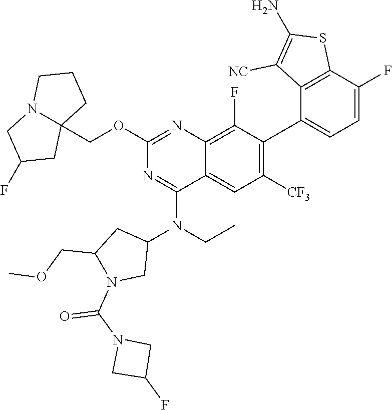 | |
| 876 | 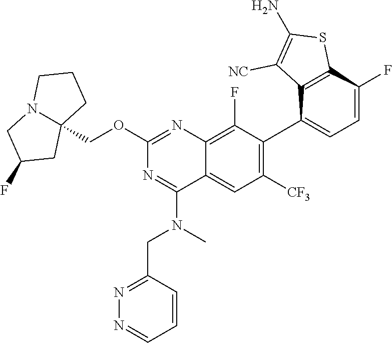 | LCMS m/z [M − 1]: 685.1 ¹H NMR (400 MHz, CD₃OD) δ 9.14-9.15 (m, 1H), 8.44 (s, 1H), 7.83-7.85 (m, 1H), 7.72-7.75 (m, 1H), 7.19-7.22 (m, 1H), 7.00 (t, 1H), 5.36 (s, 2.5H), 5.21 (s, 0.5H), 4.11-4.13 (m, 1H), 4.02-4.04 (m, 1H), 3.75 (s, 3H), 3.23 (s, 1H), 2.99-3.03 (m, 1H), 2.16-2.32 (m, 3H), 1.98-2.06 (m, 4H), 1.82-1.91 (m, 1H). |
| 876a | 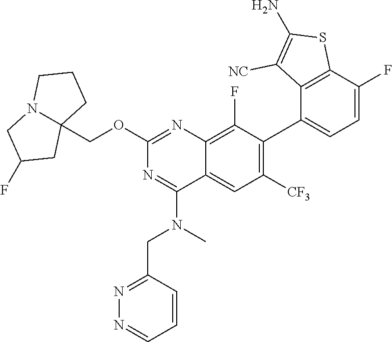 [SMILES: CN(Cc1cccnn1)c1nc(OCC23CCCN2CC(F)C3)nc2c(F)c(-c3ccc(F)c4sc(N)c(C#N)c34)c(C(F)(F)F)cc12] | |
| 877 | 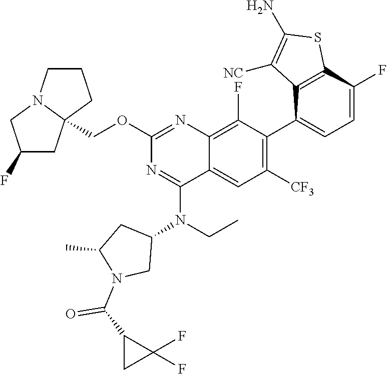 | LCMS m/z [M − 1]: 794.2 ¹H NMR (400 MHz, CD₃OD) δ 8.24 (s, 1H), 7.25-7.17 (m, 1H), 7.09 (t, 1H), 5.39-5.33 (m, 0.5H), 5.25-5.19 (m, 0.5H), 4.95-4.87 (m, 1H), 4.41-4.05 (m, 4H), 3.91- 3.85 (m, 2H), 3.28-3.15 (m, 3H), 3.07-2.86 (m, 2H), 2.82-2.61 (m, 1H), 2.38-1.71 (m, 10H), 1.57-1.38 (m, 6H). |
| 877a | 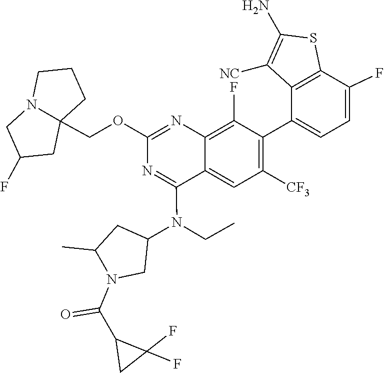 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)C2CC2(F)F)C1] | |
| 878 | 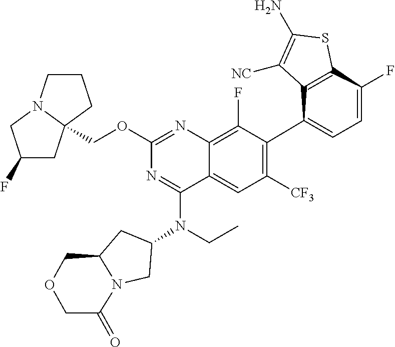 | LCMS m/z [M − 1]: 746.1 ¹H NMR (400 MHz, CD₃OD): 8.19 (s, 1 H), 7.16-7.24 (m, 1 H), 6.96-7.05 (m, 1 H), 5.39 (s, 0.5 H), 5.25 (s, 0.5 H), 4.63-4.80 (m, 2 H), 3.99-4.44 (m, 6 H), 3.80- 3.95 (m, 2 H), 3.64-3.74 (m, 1 H), 3.32-3.49 (m, 2 H), 3.20-3.28 (m, 2 H), 3.01-3.10 (m, 1 H), 1.89-2.45 (m, 8 H), 1.40-1.58 (m, 3 H). |
| 878a | 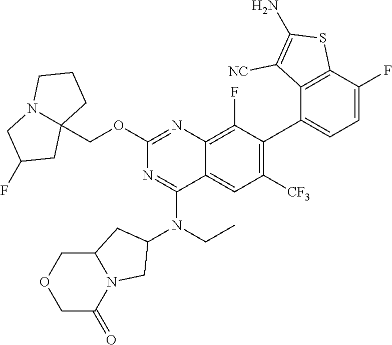 [SMILES: CCN(c1nc(OCC23CCCN2CC(F)C3)nc2c(F)c(-c3ccc(F)c4sc(N)c(C#N)c34)c(C(F)(F)F)cc12)C1CC2COCC(=O)N2C1] | |
| 879 | 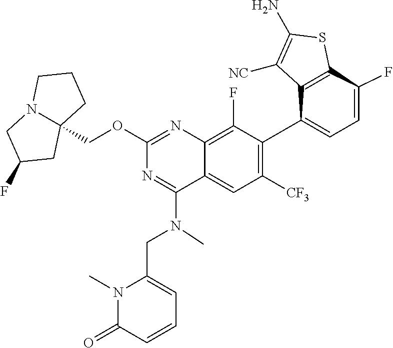 | LCMS m/z [M − 1]: 714.2 ¹H NMR (400 MHz, CD₃OD) δ 8.24 (s, 1H), 7.47-7.55 (m, 1H), 7.16-7.22 (m, 1H), 6.97-7.04 (m, 1H), 6.43- 6.58 (m, 2H), 5.31 (s, 0.5H), 5.18 (s, 0.5H), 5.10 (s, 2H), 4.10-4.22 (m, 2H), 3.57-3.66 (m, 6H), 3.11-3.21 (m, 3H), 2.92-3.00 (m, 1H), 1.80-2.20 (m, 6H). |
| 879a | 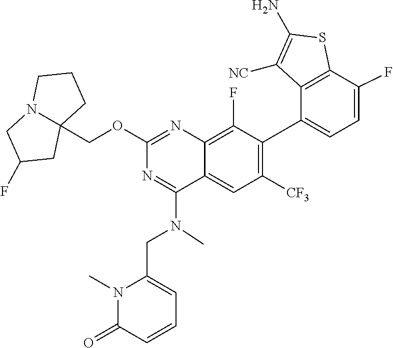 [SMILES: CN(Cc1cccc(=O)n1C)c1nc(OCC23CCCN2CC(F)C3)nc2c(F)c(-c3ccc(F)c4sc(N)c(C#N)c34)c(C(F)(F)F)cc12] | |
| 880 | 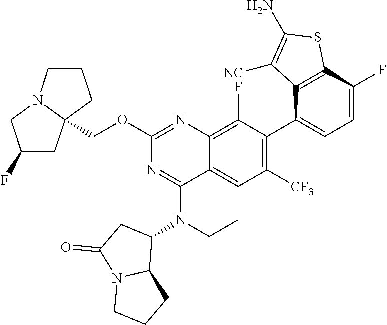 | LCMS m/z [M − 1]: 730.23 ¹H NMR (400 MHz, CD₃OD) δ 8.19-8.18 (m, 1H), 7.23-7.22 (m, 1H), 7.03-6.99 (m, 1H), 5.37 (s, 0.5H), 5.24 (s, 0.5H), 4.44-4.17 (m, 2H), 4.16-4.10 (m, 2H), 3.83-3.57 (m, 2H), 3.57-3.30 (m, 3H), 3.30-3.13 (m, 3H), 2.87-2.86 (m, 1H), 2.70-2.65 (m, 1H), 2.18-1.89 (m, 9H), 1.53-1.49 (m, 4H). |
| 880a | 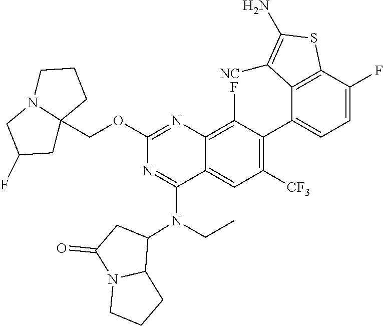 [SMILES: CCN(c1nc(OCC23CCCN2CC(F)C3)nc2c(F)c(-c3ccc(F)c4sc(N)c(C#N)c34)c(C(F)(F)F)cc12)C1CC(=O)N2CCCC12] | |
| 881 | 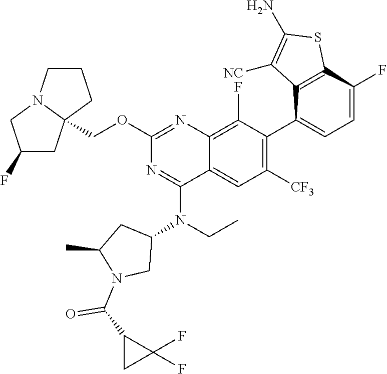 | LCMS m/z [M − 1]: 794.24 ¹H NMR (400 MHz, CD₃OD) δ 8.25-8.23 (m, 1H), 7.24-7.20 (m, 1H), 7.03-7.00 (m, 1H), 5.35-5.18 (m, 2H), 4.66-4.57 (m, 1H), 4.21-4.13 (m, 2H), 4.04-3.77 (m, 4H), 3.25-3.13 (m, 2H), 3.10-2.94 (m, 1H), 2.76- 2.70 (m, 1H), 2.70-2.67 (m, 0.5H), 2.29-2.25 (m, 0.5H), 2.14-1.89 (m, 9H), 1.46-1.25 (m, 6H). |
| 882 | 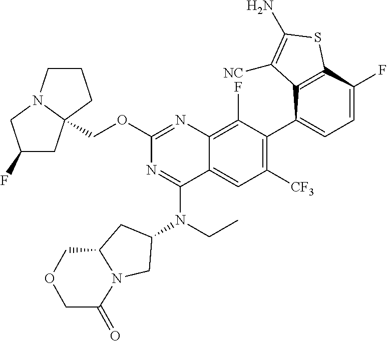 | LCMS m/z [M − 1]: 746.2 ¹H NMR (400 MHz, CD₃OD) δ 8.22 (s, 1H), 7.25-7.19 (m, 1H), 7.01 (t, 1H), 5.37 (s, 0.5H), 5.23 (s, 0.5H), 5.05-4.96 (m, 1H), 4.28-4.13 (m, 5H), 4.07-3.93 (m, 2H), 3.92-3.75 (m, 3H), 3.56 (t, 1H), 3.28-3.19 (m, 3H), 3.07-2.97 (m, 1H), 2.39-2.25 (m, 3H), 2.23-2.12 (m, 2H), 2.02-1.86 (m, 3H), 1.49 (t, 3H). |
| 883 | 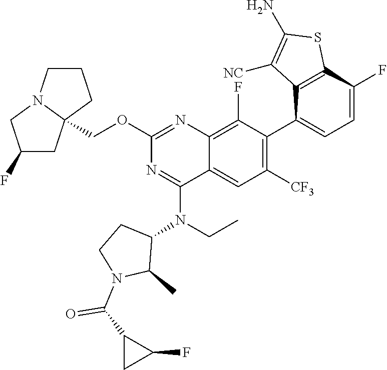 | LCMS m/z [M − 1]: 776.3 ¹H NMR (400 MHz, CD₃OD) δ 8.23-8.21 (m, 1H), 7.24-7.20 (m, 1H), 7.01 (t, 1H), 5.36 (s, 0.5H), 5.23 (s, 0.5H), 4.96 (s, 0.5H), 4.82-4.77 (m, 0.5H), 4.73-4.66 (m, 1.5H), 44.44-4.41 (m, 0.5H), 4.27-4.21 (m, 2H), 4.19-4.13 (m, 1H), 4.07-3.90 (m, 1H), 3.87-3.76 (m, 2H), 3.49-3.32 (m, 1H), 3.25-3.19 (m, 2H), 3.04-2.97 (m, 1H), 2.68-2.55 (m, 1H), 2.46-2.32 (m, 2H), 2.27- 2.07 (m, 3H), 2.01-1.94 (m, 2H), 1.91-1.83 (m, 1H), 1.52-1.46 (m, 3H), 1.43-1.29 (m, 5H). |
| 884 | 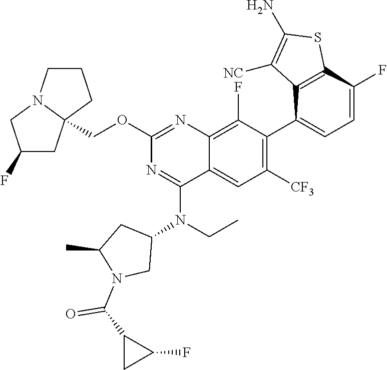 | LCMS m/z [M − 1]: 776.2 ¹H NMR (400 MHz, CD₃OD) δ = 8.24 (d, J = 5.4, 1H), 7.25-7.18 (m, 1H), 7.01 (t, J = 8.9, 1H), 5.44-5.14 (m, 2H), 5.00-4.91 (m, 0.5H), 4.78 (dd, J = 6.2, 2.8, 0.5H), 4.65-4.42 (m, 1H), 4.28 (dt, J = 19.0, 10.5, 2.5H), 4.01- 3.73 (m, 3.5H), 3.43-3.33 (m, 0.5H), 3.26 (dd, J = 13.4, 4.1, 2.5H), 3.10-2.97 (m, 1H), 2.88-2.65 (m, 1H), 2.37-1.70 (m, 9H), 1.49-1.30 (m, 6H), 1.17- 1.01 (m, 1H). |
| 885 | 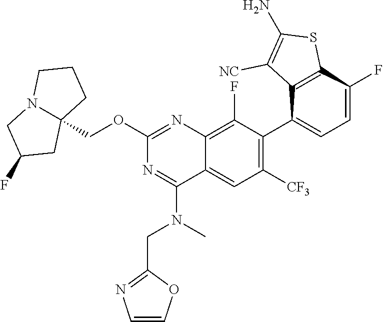 | LCMS m/z [M − 1]: 674.1 ¹H NMR (400 MHz, CD₃OD) δ 8.44 (s, 1H), 7.95 (s, 1H), 7.22-7.19 (m, 2H), 7.00 (t, 1H), 5.36 (s, 0.5H), 5.23 (s, 0.5H), 5.20-5.08 (m, 2H), 4.22-4.13 (m, 2H), 3.70 (s, 3H), 3.27-3.19 (m, 3H), 3.05-2.99 (m, 1H), 2.26-2.14 (m, 2H), 2.10-2.08 (m, 1H), 2.02-1.94 (m, 2H), 1.92-1.84 (m, 1H). |
| 885a | 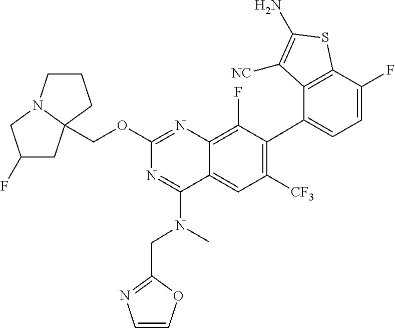 [SMILES: CN(Cc1ncco1)c1nc(OCC23CCCN2CC(F)C3)nc2c(F)c(-c3ccc(F)c4sc(N)c(C#N)c34)c(C(F)(F)F)cc12] | |
| 886 | 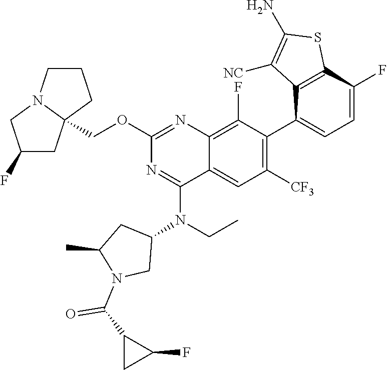 | LCMS m/z [M − 1]: 776.2 ¹H NMR (400 MHz, CD₃OD) δ = 8.24 (d, J = 13.2, 1H), 7.22 (dd, J = 8.3, 5.1, 1H), 7.01 (t, J = 8.9, 1H), 5.28 (dd, J = 39.8, 24.2, 2H), 4.83 (d, J = 4.9, 0.5H), 4.68 (d, J = 20.6, 0.5H), 4.61-4.37 (m, 1H), 4.34-4.18 (m, 2.5H), 3.98-3.67 (m, 3.5H), 3.30-3.19 (m, 3H), 3.02 (dd, J = 14.7, 9.5, 1H), 2.82-2.63 (m, 1H), 2.53-1.65 (m, 9H), 1.49-1.38 (m, 5.5H), 1.25 (d, J = 6.5, 1.5H). |
| 887 | 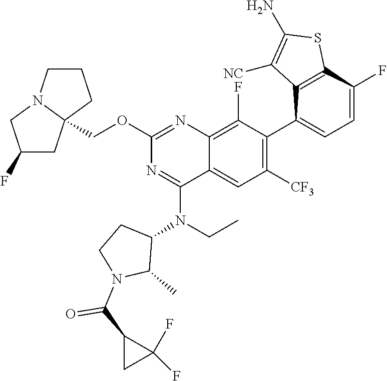 | LCMS m/z [M − 1]: 794.3 ¹H NMR (400 MHz, CD₃OD) δ 8.38 (s, 1H), 7.25-7.20 (m, 1H), 7.03 (t, 1H), 5.65-5.60 (m, 0.5H), 5.51-5.47 (m, 0.5H), 5.36-5.31 (m, 0.5H), 5.06-5.03 (m, 0.5H), 4.77-4.69 (m, 1H), 4.61-4.52 (m, 1H), 4.17-3.77 (m, 6H), 3.47-3.40 (m, 1H), 2.91-2.70 (m, 2H), 2.66-2.56 (m, 2H), 2.42-1.97 (m, 7H), 1.85-1.77 (m, 1H), 1.65- 1.55 (m, 1H), 1.51-1.40 (m, 3H), 1.11-0.95 (m, 3H). |
| 887a | 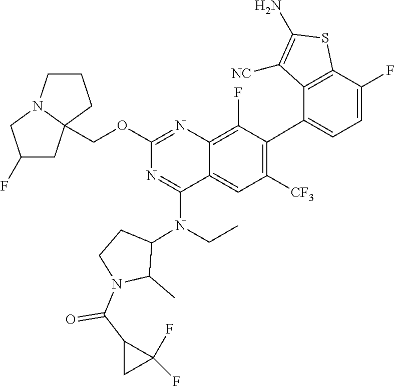 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CC2(F)F)C1C] | |
| 888 | 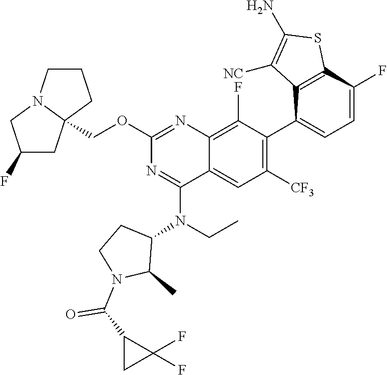 | LCMS m/z [M − 1]: 794.2 ¹H NMR (400 MHz, CD₃OD) δ 8.21 (s, 1H), 7.20-7.24 (m, 1H), 7.01 (t, 1H), 5.37 (s, 0.5H), 5.23 (s, 0.5H), 4.52-4.61 (m, 2H), 4.16-4.30 (m, 3H), 3.78-3.90 (m, 2H), 3.68-3.74 (m, 1H), 3.17-3.25 (m, 3H), 2.94-3.04 (m, 2H), 2.56-2.63 (m, 1H), 2.43-2.53 (m, 1H), 2.26- 2.35 (m, 1H), 2.08-2.22 (m, 2H), 1.94-2.02 (m, 3H), 1.74-1.90 (m, 2H), 1.43-1.49 (m, 3H), 1.36-1.40 (m, 3H). |
| 889 | 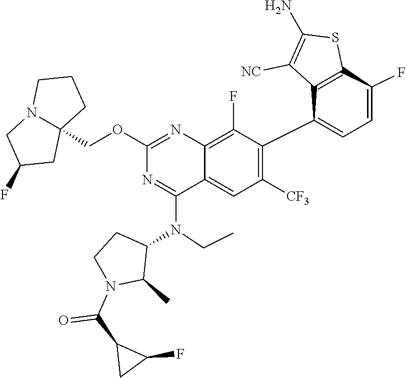 | LCMS m/z [M − 1]: 776.2 ¹H NMR (400 MHz, CD₃OD) δ 8.22 (s, 1H), 7.20-7.24 (m, 1H), 7.01 (t, 1H), 5.38 (s, 0.5H), 5.25 (s, 0.5H), 5.00-5.04 (m, 0.5H), 4.90-4.94 (m, 0.5H), 4.47-4.79 (m, 3H), 4.24-4.35 (m, 2H), 4.06-4.13 (m, 1H), 3.76-3.95 (m, 3H), 3.35-3.49 (m, 1H), 3.23-3.26 (m, 1H), 3.01- 3.07 (m, 1H), 2.45-2.63 (m, 2H), 2.35-2.39 (m, 1H), 2.11-2.28 (m, 3H), 1.97-2.05 (m, 2H), 1.84-1.94 (m, 1H), 1.68-1.79 (m, 1H), 1.34-1.94 (m, 6H), 1.07-1.12 (m, 1H). |
| 890 | 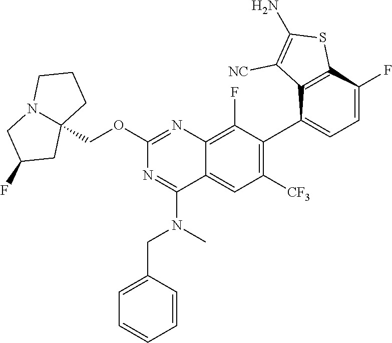 | LCMS m/z [M − 1]: 683.1 ¹H NMR (400 MHz, CD₃OD) δ 8.23 (s, 1H), 7.30-7.47 (m, 5H), 7.15-7.21 (m, 1H), 6.98 (t, 1H), 5.34 (s, 0.5H), 5.20 (s, 0.5H), 5.05-5.19 (m, 2H), 4.25-4.32 (m, 1H), 4.18-4.24 (m, 1H), 3.50 (s, 3H), 3.17-3.29 (m, 3H), 2.96-3.05 (m, 1H), 2.17-2.36 (m, 2H), 2.08-2.16 (m, 1H), 1.92-2.02 (m, 2H), 1.81-1.91 (m, 1H). |
| 890a | 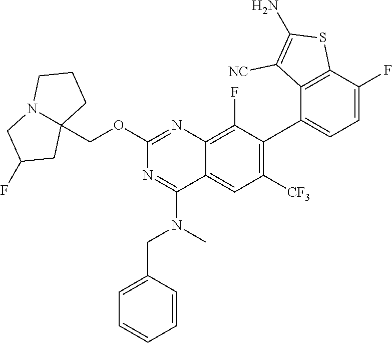 [SMILES: CN(Cc1ccccc1)c1nc(OCC23CCCN2CC(F)C3)nc2c(F)c(-c3ccc(F)c4sc(N)c(C#N)c34)c(C(F)(F)F)cc12] | |
| 891 | 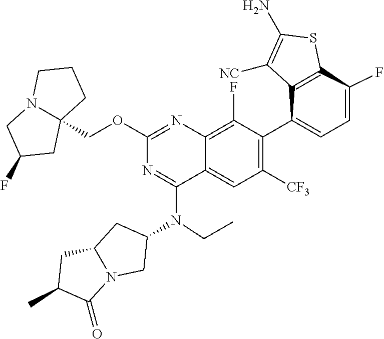 | LCMS m/z [M − 1]: 744.3 ¹H NMR (400 MHz, CD₃OD) δ 8.29-8.14 (m, 1H), 7.29-7.17 (m, 1H), 7.01 (t, J = 8.9 Hz, 1H), 5.37 (s, 1H), 5.35-5.13 (m, 2H), 4.36-4.12 (m, 2H), 4.03- 3.92 (m, 1H), 3.90-3.54 (m, 4H), 3.28-3.19 (m, 2H), 3.09-2.97 (m, 1H), 2.94-2.82 (m, 1H), 2.71-2.54 (m, 1H), 2.32 (m, 4H), 2.11-1.84 (m, 4H), 1.59 (m, 1H), 1.44 (t, J = 6.9 Hz, 3H), 1.21 (d, J = 7.1 Hz, 2H). |
| 892 | 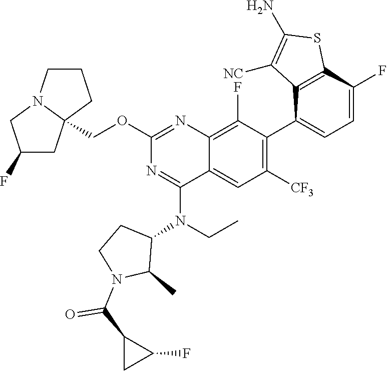 | LCMS m/z [M − 1]: 776.4 ¹H NMR (400 MHz, CD₃OD) δ 8.22 (s, 1H), 7.25-7.20 (m, 1H), 7.01 (t, 1H), 5.37 (s, 0.5H), 5.23 (s, 0.5H), 4.92-4.88 (m, 1H), 4.78-4.62 (m, 2H), 4.49-4.44 (m, 0.5H), 4.32-4.19 (m, 2H), 4.17-4.12 (m, 0.5H), 4.03- 3.77 (m, 3H), 3.52-3.34 (m, 1H), 3.25-3.17 (m, 2H), 3.06-2.97 (m, 1H), 2.63-2.52 (m, 1H), 2.47-2.08 (m, 5H), 2.02-1.86 (m, 3H), 1.54-1.41 (m, 5H), 1.34-1.28 (m, 3H). |
| 893 | 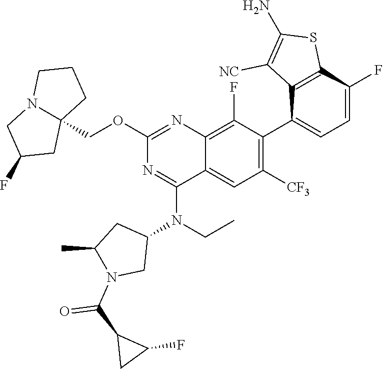 | LCMS m/z [M − 1]: 776.2 ¹H NMR (400 MHz, CD₃OD) δ 8.47 (brs, 1H), 8.28 (d, J = 9.0 Hz, 1H), 7.23 (m, 1H), 7.01 (t, J = 8.8 Hz, 1H), 5.37-5.24 (d, 1H), 5.31-5.22 (m, 1H), 4.93-4.89 (m, 1H), 4.61-4.37 (m, 3H), 4.29-3.57 (m, 7H), 3.31-3.20 (m, 1H), 2.65-2.01 (m, 9H), 1.47-1.25 (m, 8H). |
| 894 | 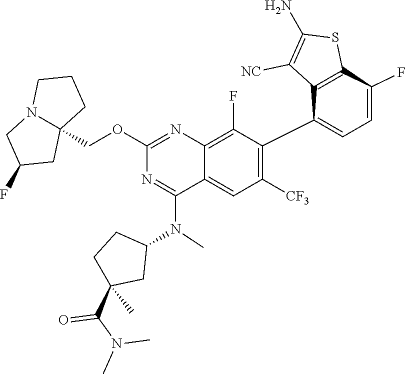 | LCMS m/z [M − 1]: 760.2 ¹H NMR (400 MHz, CD₃OD) δ 8.23 (s, 1H), 7.23-7.20 (m, 1H), 7.01 (t, J = 8.8 Hz, 1H), 5.45-5.31 (d, 1H), 4.82- 4.80 (m, 1H), 4.41 (s, 1H), 3.87-3.81 (m, 2H), 3.59- 3.35 (m, 3H), 3.09-2.91 (m, 7H), 2.46-1.75 (m, 12H), 1.56-1.41 (m, 6H). |
| 895 | 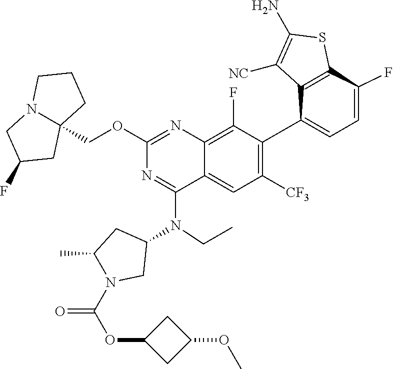 | LCMS m/z [M − 1]: 818.3 ¹H NMR (400 MHz, CD₃OD) δ 8.23 (s, 1H), 7.18-7.25 (m, 1H), 7.01 (t, 1H), 5.37 (s, 0.5H), 5.24 (s, 0.5H), 5.01-5.09 (m, 1H), 4.79-4.85 (m, 1H), 4.33 (d, 1H), 4.26 (d, 1H), 4.03-4.16 (m, 2H), 3.80-4.02 (m, 3H), 3.46-3.62 (m, 1H), 3.27-3.30 (m, 2H), 3.23 (s, 3H), 2.99-3.08 (m, 1H), 2.61-2.85 (m, 1H), 2.57 (s, 1H), 2.22-2.41 (m, 5H), 1.82-2.21 (m, 6H), 1.42 (t, 6H). |
| 895a | 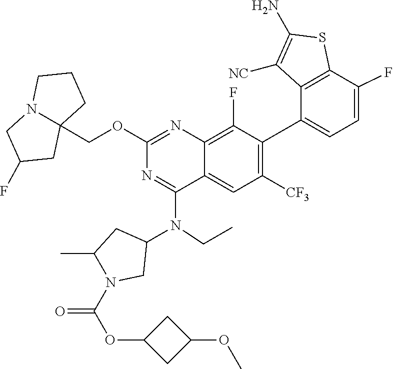 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)OC2CC(OC)C2)C1] | |
| 896 | 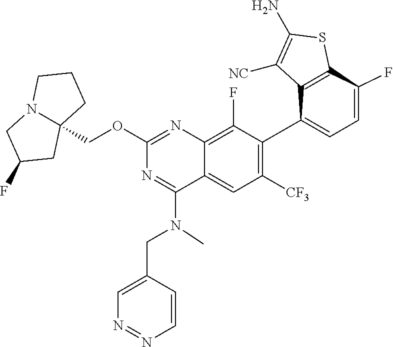 | LCMS m/z [M − 1]: 685.2 ¹H NMR (400 MHz, CD₃OD) δ 9.30 (s, 1H), 9.14 (d, 1H), 8.40 (s, 1H), 7.34-7.76 (m, 1H), 7.18-7.23 (m, 1H), 7.01 (t, 1H), 5.31 (s, 0.5H), 5.20-5.24 (m, 1H), 5.19 (s, 0.5H), 5.11-5.16 (m, 1H), 4.09 (d, 1H), 4.00 (d, 1H), 3.71 (s, 3H), 3.14-3.25 (m, 3H), 2.95-3.03 (m, 1H), 2.17-2.25 (m, 1H), 2.01-2.15 (m, 1H), 1.99-2.05 (m, 1H), 1.90-1.98 (m, 2H), 1.74-1.83 (m, 1H). |
| 896a | 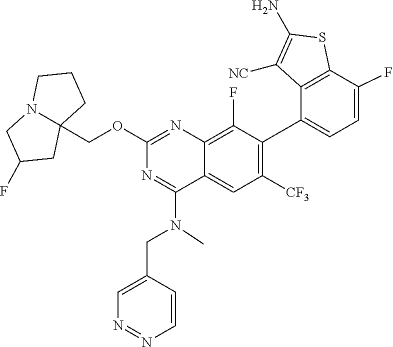 [SMILES: CN(Cc1ccnnc1)c1nc(OCC23CCCN2CC(F)C3)nc2c(F)c(-c3ccc(F)c4sc(N)c(C#N)c34)c(C(F)(F)F)cc12] | |
| 897 | 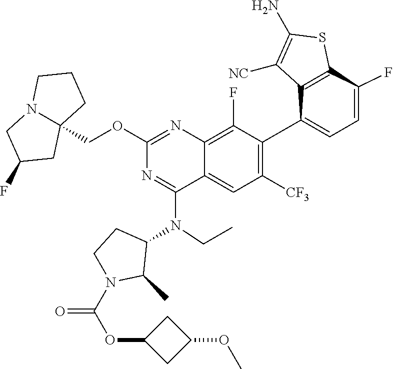 | LCMS m/z [M − 1]: 818.3 ¹H NMR (400 MHz, CD₃OD) δ 8.21 (s, 1H), 7.19-7.25 (m, 1H), 7.01 (t, 1H), 5.37 (s, 0.5H), 5.23 (s, 0.5H), 5.03-5.11 (m, 1H), 4.60-4.67 (m, 1H), 4.18-4.33 (m, 3H), 4.08 (s, 1H), 3.76-3.95 (m, 3H), 3.40-3.52 (m, 1H), 3.18-3.27 (m, 5H), 2.98-3.06 (m, 1H), 2.62-2.87 (m, 1H), 2.45-2.57 (m, 1H), 2.06-2.41 (m, 8H), 1.94-2.03 (m, 2H), 1.83-1.93 (m, 1H), 1.31-1.50 (m, 6H). |
| 897a | 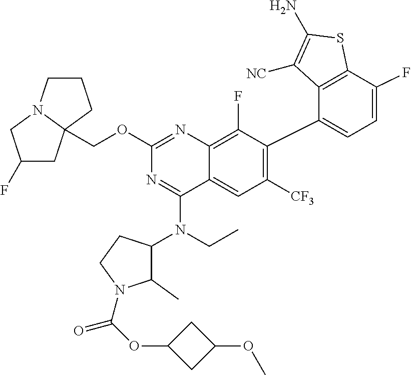 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC2CC(OC)C2)C1C] | |
| 898 | 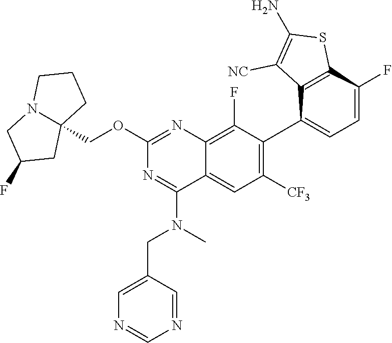 | LCMS m/z [M − 1]: 685.1 ¹H NMR (400 MHz, CD₃OD) δ 9.11 (s, 1H), 8.91 (s, 2H), 8.38 (s, 1H), 7.22-7.19 (m, 1H), 7.00 (t, 1H), 5.33 (s, 0.5H), 5.20-5.16 (m, 1.5H), 5.12-5.08 (m, 1H), 4.21- 4.18 (m, 1H), 4.12-4.09 (m, 1H), 3.67 (s, 3H), 3.25-3.16 (m, 3H), 2.99-2.95 (m, 1H), 2.30-2.22 (m, 1H), 2.18- 2.16 (m, 1H), 2.09-2.05 (m, 1H), 1.98-1.91 (m, 2H), 1.84-1.77 (m, 1H). |
| 898a | 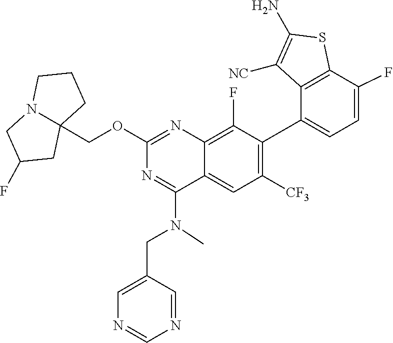 [SMILES: CN(Cc1cncnc1)c1nc(OCC23CCCN2CC(F)C3)nc2c(F)c(-c3ccc(F)c4sc(N)c(C#N)c34)c(C(F)(F)F)cc12] | |
| 899 | 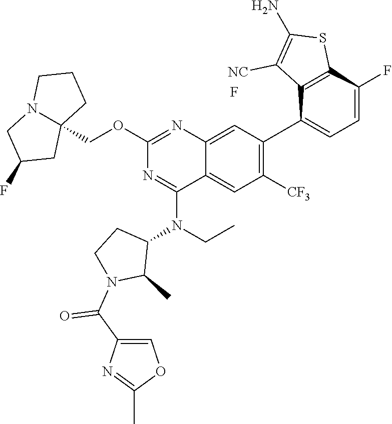 | LCMS m/z [M − 1]: 799.2 ¹H NMR (400 MHz, CD₃OD) δ 8.29 (s, 1H), 8.23-8.22 (m, 1H), 7.24-7.21 (m, 1H), 7.01 (t, 1H), 5.34 (s, 0.5H), 5.21 (s, 0.5H), 4.78-4.58 (m, 2H), 4.43-4.17 (m, 3H), 3.96-3.76 (m, 3H), 3.21-3.17 (m, 2H), 3.03-2.94 (m, 1H), 2.67-2.55 (m, 1H), 2.49-2.37 (m, 4H), 2.33-2.16 (m, 2H), 2.15-2.01 (m, 2H), 1.97-1.80 (m, 3H), 1.49- 1.32 (m, 6H). |
| 899a | 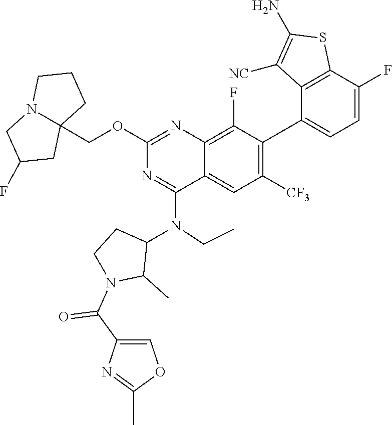 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2coc(C)n2)C1C] | |
| 900 | 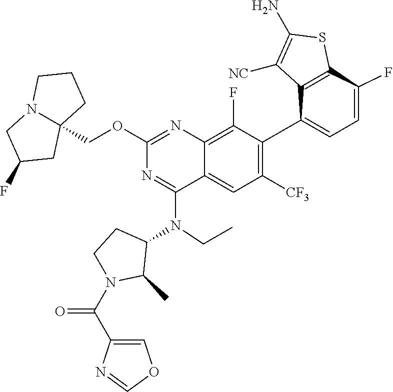 | LCMS m/z [M − 1]: 785.2 ¹H NMR (400 MHz, CD₃OD) δ 8.47 (s, 1H), 8.26-8.22 (m, 2H), 7.24-7.21 (m, 1H), 7.01 (t, 1H), 5.35 (s, 0.5H), 5.22 (s, 0.5H), 4.76-4.62 (m, 2H), 4.47-4.21 (m, 3H), 3.93-3.73 (m, 3H), 3.72-3.55 (m, 1H), 3.25-3.17 (m, 2H), 3.00 (s, 1H), 2.68-2.43 (m, 2H), 2.31-2.12 (m, 2H), 2.08-1.88 (m, 3H), 1.87-1.77 (m, 1H), 1.67-1.29 (m, 6H). |
| 900a | 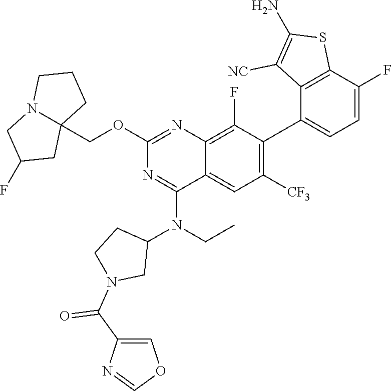 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2cocn2)C1] | |
| 901 | 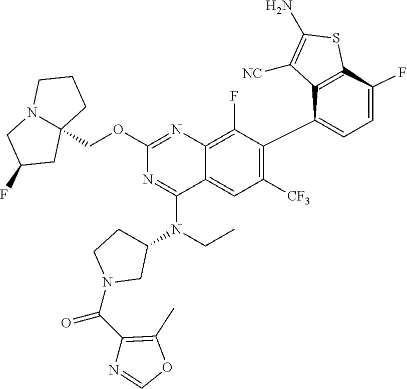 | LCMS m/z [M − 1]: 785.2 ¹H NMR (400 MHz, CD₃OD) δ 8.25 (d, 1H), 8.02 (d, 1H), 7.20-7.23 (m, 1H), 7.01 (t, 1H), 5.34 (s, 0.5H), 5.20 (s, 0.5H), 5.01-5.09 (m, 1H), 4.38-4.56 (m, 1H), 4.09-4.25 (m, 3H), 3.77-3.98 (m, 3H), 3.47-3.66 (m, 1H), 3.13-3.21 (m, 3H), 2.95-3.01 (m, 1H), 2.35-2.58 (m, 5H), 2.04-2.28 (m, 3H), 1.81-1.95 (m, 3H), 1.47 (t, 3H). |
| 901a | 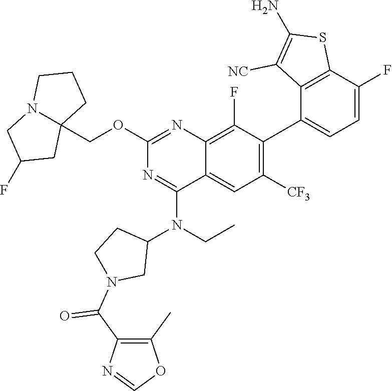 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2ncoc2C)C1] | |
| 902 | 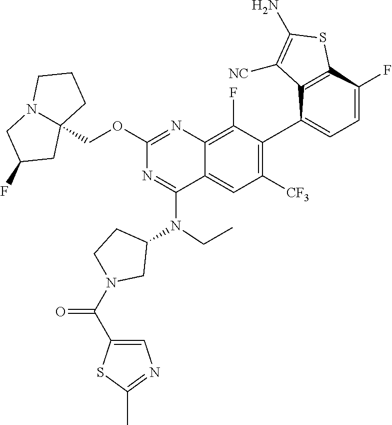 | LCMS m/z [M − 1]: 801.2 ¹H NMR (400 MHz, CD₃OD) δ 8.51 (brs, 0.3H), 8.26 (s, 1H), 8.12 (d, J = 13.2 Hz, 1H), 7.32-7.16 (m, 1H), 7.01 (t, J = 9.0 Hz, 1H), 5.37-5.29 (s, 0.55H), 5.25-5.16 (m, 0.54H), 5.15-4.98 (m, 1H), 4.44-3.57 (m, 8H), 3.28- 2.95 (m, 4H), 2.79-2.60 (m, 3H), 2.69-1.80 (m, 8H), 1.69-1.39 (m, 3H). |
| 902a | 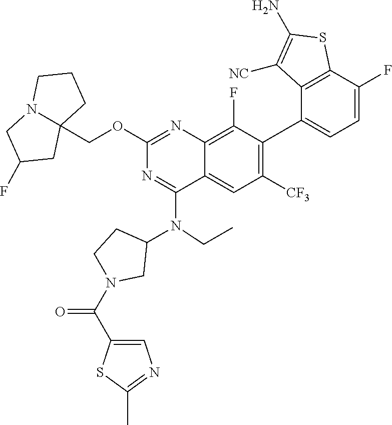 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2cnc(C)s2)C1] | |
| 903 | 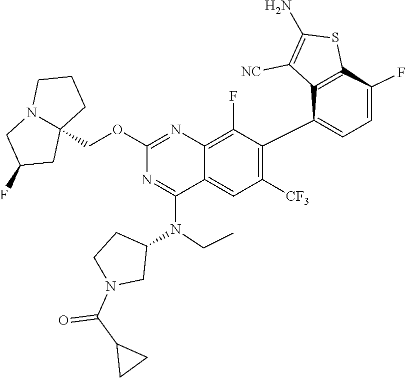 | LCMS m/z [M − 1]: 774.2 ¹H NMR (400 MHz, CD₃OD) δ 8.25 (d, 1H), 7.20-7.23 (m, 1H), 7.01 (t, 1H), 5.37 (s, 0.5H), 5.23 (s, 0.5H), 5.00-5.12 (m, 1H), 4.22-4.30 (m, 2H), 3.85-4.11 (m, 4H), 3.58-3.76 (m, 1H), 3.39-3.47 (m, 1H), 3.12-3.24 (m, 3H), 2.96-3.04 (m, 1H), 2.33-2.58 (m, 3H), 2.09- 2.26 (m, 2H), 1.81-2.00 (m, 4H), 1.43-1.50 (m, 3H), 0.83-0.93 (m, 4H). |
| 903a | 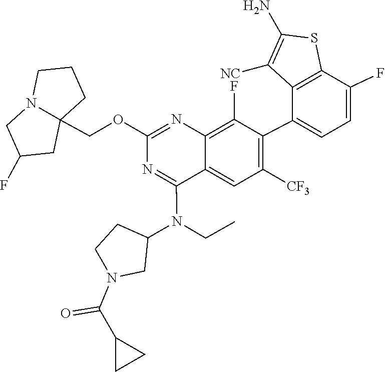 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CC2)C1] | |
| 904 | 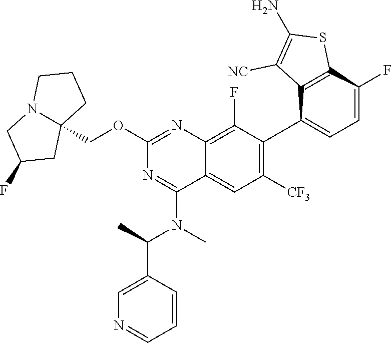 | LCMS m/z [M − 1]: 698.2 ¹H NMR (400 MHz, CD₃OD) δ 8.63 (s, 1H), 8.50 (d, 1H), 8.31 (s, 1H), 7.94 (t, 1H), 7.47-7.50 (m, 1H), 7.19- 7.23 (m, 1H), 7.01 (t, 1H), 6.26-6.33 (m, 1H), 5.35 (s, 0.5H), 5.21 (s, 0.5H), 4.16-4.25 (m, 2H), 3.35-3.48 (m, 2H), 3.16-3.26 (m, 3H), 3.06-3.35 (m, 1H), 2.95-3.02 (m, 1H), 2.13-2.36 (m, 3H), 2.06-2.02 (m, 1H), 1.91- 1.99 (m, 2H), 1.84 (d, 3H). |
| 904a | 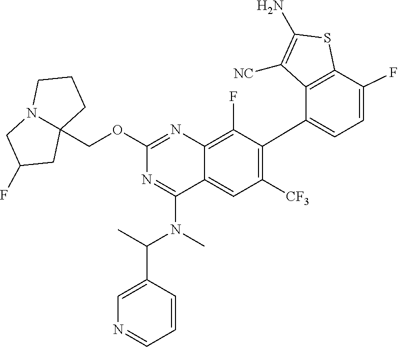 [SMILES: CC(c1cccnc1)N(C)c1nc(OCC23CCCN2CC(F)C3)nc2c(F)c(-c3ccc(F)c4sc(N)c(C#N)c34)c(C(F)(F)F)cc12] | |
| 905 | 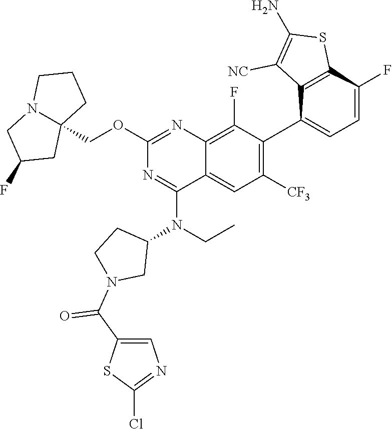 | LCMS m/z [M − 1]: 821.0 ¹H NMR (400 MHz, CD₃OD) δ 8.27-8.12 (m, 1H), 8.12-8.08 (m, 1H), 7.23-7.21 (m, 1H), 7.03-6.99 (m, 1H), 5.31 (s, 0.5H), 5.18 (s, 0.5H), 5.05-4.87 (m, 1H), 4.23 (m, 0.5H), 4.16-4.01 (m, 3H), 3.89-3.85 (m, 4H), 3.66 (m, 0.5H), 3.31-3.16 (m, 3H), 3.16-2.96 (m, 1H), 2.53-2.34 (m, 2H), 2.14-1.82 (m, 6H), 1.44-1.08 (m, 3H). |
| 905a | 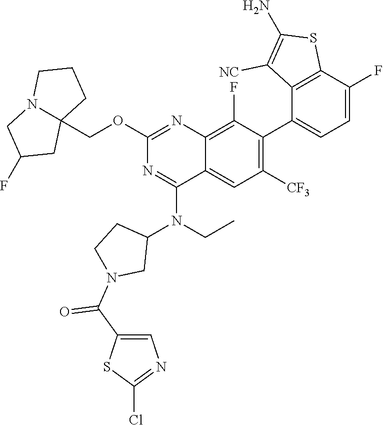 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2cnc(Cl)s2)C1] | |
| 906 | 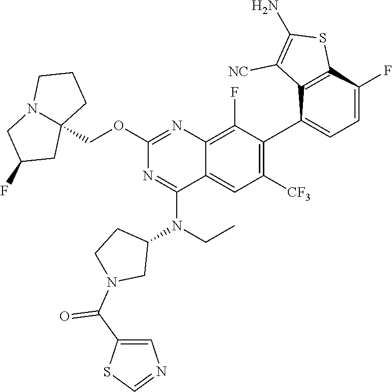 | LCMS m/z [M − 1]: 787.2 ¹H NMR (400 MHz, CD₃OD) δ 9.17-9.15 (m, 1H), 8.54-8.42 (m, 1H), 8.38-8.26 (S, 1H), 7.23-7.21 (m, 1H), 7.03-6.99 (m, 1H), 5.32 (s, 0.5H), 5.28 (s, 0.5H), 5.19-5.08 (m, 1H), 4.28 (m, 0.5H), 4.28-4.18 (m, 3H), 4.00-3.89 (m, 4H), 3.69 (m, 0.5H), 3.31-3.14 (m, 3H), 2.64 (s, 1H), 2.64-2.22 (m, 2H), 2.13-1.82 (m, 6H), 1.49 (s, 3H). |
| 906a | 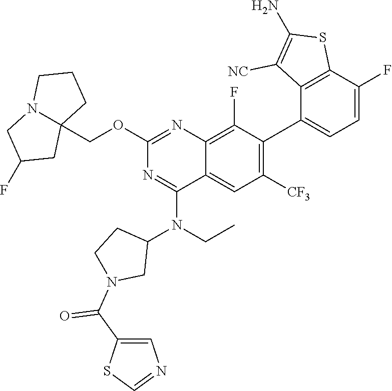 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2cncs2)C1] | |
| 907 | 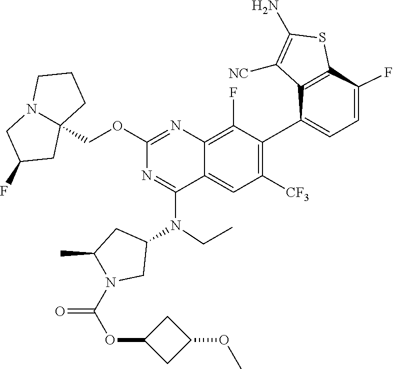 | LCMS m/z [M − 1]: 818.3 ¹H NMR (400 MHz, CD₃OD) δ 8.23 (s, 1H), 7.29-7.15 (m, 1H), 7.07-6.94 (m, 1H), 5.40 (s, 0.5H), 5.30- 5.12 (m, 1.5H), 5.06 (s, 1H), 4.38-4.25 (m, 2H), 4.20 (s, 1H), 4.08 (s, 1H), 3.96-3.72 (m, 3H), 3.69-3.57 (m, 1H), 3.47-3.31 (m, 2H), 3.30-3.17 (m, 5H), 3.11- 2.98 (m, 1H), 2.73-2.56 (m, 1H), 2.46-2.20 (m, 5H), 2.18-1.82 (m, 5H), 1.43 (t, J = 6.8 Hz, 3H), 1.28 (s, 3H). |
| 908 | 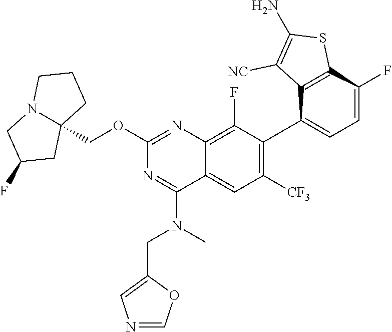 | LCMS m/z [M − 1]: 673.0 ¹H NMR (400 MHz, CD₃OD) δ = 8.40 (s, 1H), 8.25 (s, 1H), 7.29 (s, 1H), 7.21 (dd, J = 8.3, 5.1, 1H), 7.01 (t, J = 8.9, 1H), 5.37 (s, 0.5H), 5.24 (s, 0.5H), 5.13 (d, J = 4.9 2H), 4.29 (dd, J = 34.2, 10.6, 2H), 3.57 (s, 3H), 3.24 (d, J = 19.3, 3H), 3.05-2.98 (m, 1H), 2.24 (dd, J = 14.3, 10.3, 3H), 2.00 (dd, J = 10.6, 6.4, 3H). |
| 908a | 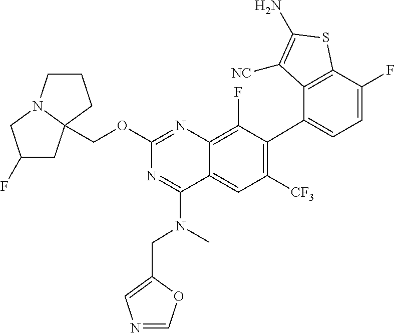 [SMILES: CN(Cc1cnco1)c1nc(OCC23CCCN2CC(F)C3)nc2c(F)c(-c3ccc(F)c4sc(N)c(C#N)c34)c(C(F)(F)F)cc12] | |
| 909 | 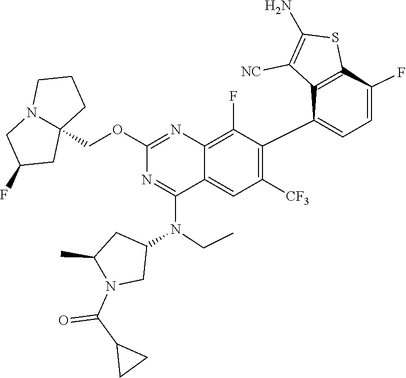 | LCMS m/z [M − 1]: 758.3 ¹H NMR (400 MHz, CD₃OD) δ 8.25 (d, J = 10.9 Hz, 1H), 7.24-7.21 (m, 1H), 7.04-6.99 (m, 1H), 5.38-5.24 (m, 2H), 4.60-4.58 (m, 1H), 4.41-4.39 (m, 1H), 4.37- 4.21 (m, 2H), 3.98-3.74 (m, 3H), 3.64-3.48 (m, 1H), 3.24-3.20 (m, 2H), 3.04-2.99 (m, 1H), 2.81-2.65 (m, 2H), 2.37-2.05 (m, 4H), 2.00-1.77 (m, 4H), 1.47-1.41 (m, 3H), 1.29-1.27 (m, 3H), 1.00-0.83 (m, 4H). |
| 909a | 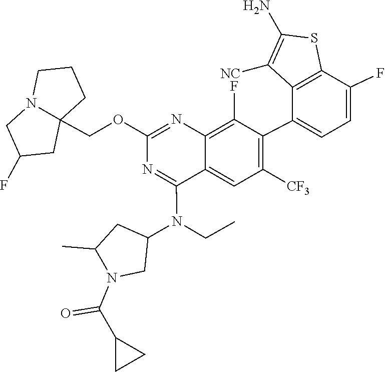 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)C2CC2)C1] | |
| 910 | 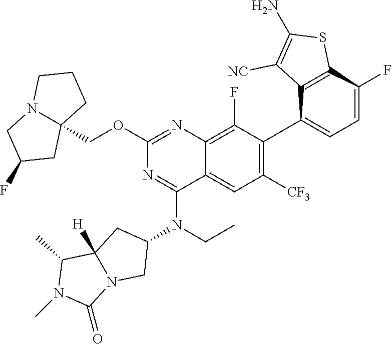 | LCMS m/z [M − 1]: 759.2 ¹H NMR (400 MHz, CD₃OD) δ 8.19 (s, 1H), 7.19-7.21 (m, 1H), 7.01 (t, 1H), 5.35 (s 0.5H), 5.22 (s, 0.5H), 5.16-5.20 (m, 1H), 4.31 (d, 1H), 4.20 (d, 1H), 3.78 (t, 1H), 3.54-3.67 (m, 5H), 3.12-3.29 (m, 3H), 2.98 (s, 1H), 2.66 (d, 2H), 2.23-2.37 (m, 1H), 2.31-2.35 (m, 3H), 1.82- 2.28 (m, 5H), 1.28-1.48 (m, 6H). |
| 910a | 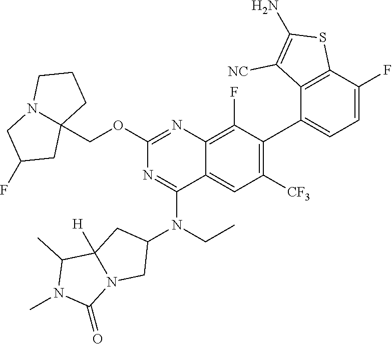 [SMILES: CCN(c1nc(OCC23CCCN2CC(F)C3)nc2c(F)c(-c3ccc(F)c4sc(N)c(C#N)c34)c(C(F)(F)F)cc12)C1CC2C(C)N(C)C(=O)N2C1] | |
| 911 | 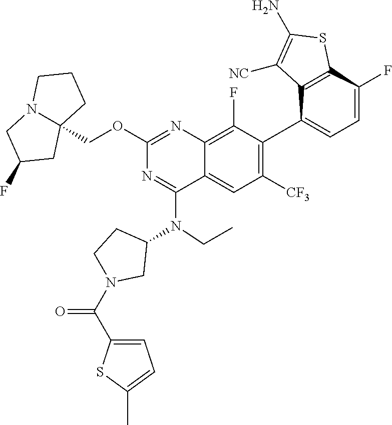 | LCMS m/z [M − 1]: 800.2 ¹H NMR (400 MHz, CD₃OD) δ 8.27 (s, 1H), 7.49 (s, 1H), 7.25-7.19 (m, 1H), 7.02 (t, J = 8.9 Hz, 1H), 6.85 (s, 1H), 5.30 (d, J = 20.9 Hz, 0.5H), 5.14 (d, J = 20.9 Hz, 0.5H), 5.10-5.00 (m, 1H), 4.58-4.08 (m, 4H), 4.05- 3.80 (m, 4H), 3.68 (s, 1H), 3.24-3.13 (m, 2H), 2.99 (s, 1H), 2.70-2.39 (m, 5H), 2.30-2.01 (m, 3H), 2.00- 1.77 (m, 3H), 1.51 (s, 3H). |
| 911a | 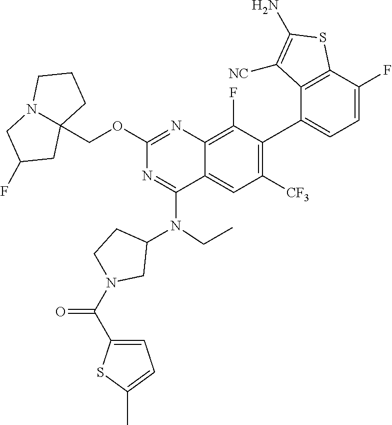 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2ccc(C)s2)C1] | |
| 912 | 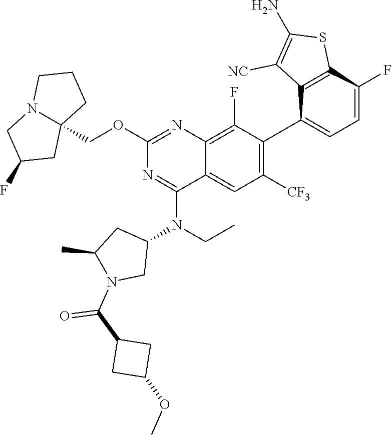 | LCMS m/z [M − 1]: 802.3 ¹H NMR (400 MHz, CD₃OD) δ 8.25 (s, 1H), 7.19-7.23 (m, 1H), 7.01 (t, 1H), 5.37 (s, 0.5H), 5.24 (s, 0.5H), 5.18-5.22 (m, 1H), 4.20-4.43 (m, 3H), 3.57-4.16 (m, 6H), 3.26 (s, 2H), 3.23 (s, 3H), 2.99-3.03 (m, 1H), 2.33-2.69 (m, 4H), 2.10-2.28 (m, 5H), 1.87-2.06 (m, 4H), 1.39-1.45 (m, 3H), 1.26-1.33 (m, 3H). |
| 912a | 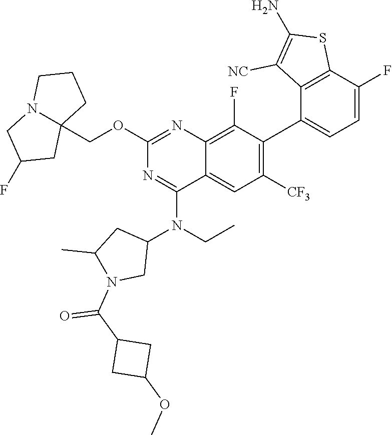 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)C2CC(OC)C2)C1] | |
| 913 | 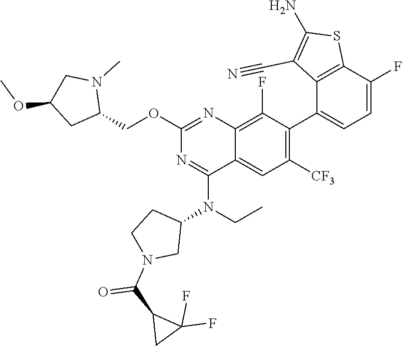 [SMILES: CCN(c1nc(OC[C@@H]2C[C@@H](OC)CN2C)nc2c(F)c(-c3ccc(F)c4sc(N)c(C#N)c34)c(C(F)(F)F)cc12)[C@H]1CCN(C(=O)[C@@H]2CC2(F)F)C1] | LCMS m/z [M − 1]: 768.2 ¹H NMR (400 MHz, CD₃OD) δ 8.31 (d, J = 11.2 Hz, 1H), 7.28-7.19 (m, 1H), 7.03 (t, J = 8.8 Hz, 1H), 5.18- 4.93 (m, 2H), 4.75-4.57 (m, 1H), 4.27-3.61 (m, 9H), 3.50-3.35 (m, 4H), 3.13 (s, 3H), 2.94-2.80 (m, 1H), 2.69-2.40 (m, 3H), 2.22-2.01 (m, 2H), 1.90- 1.73 (m, 1H), 1.59-1.42 (m, 3H). |
| 913a | 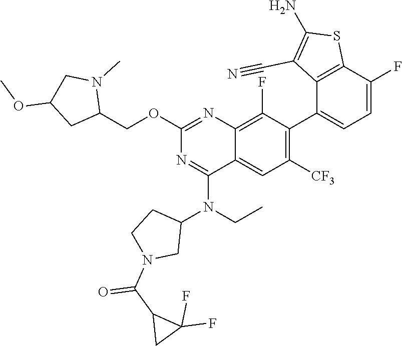 [SMILES: CCN(c1nc(OCC2CC(OC)CN2C)nc2c(F)c(-c3ccc(F)c4sc(N)c(C#N)c34)c(C(F)(F)F)cc12)C1CCN(C(=O)C2CC2(F)F)C1] | |
| 914 | 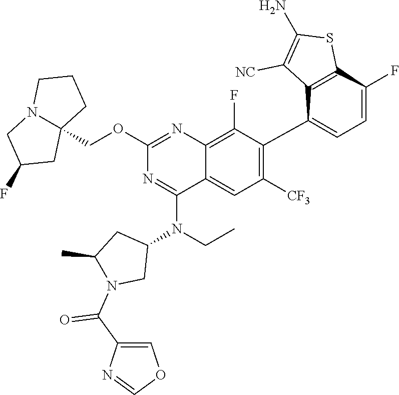 | LCMS m/z [M − 1]: 785.2 ¹H NMR (400 MHz, CD₃OD) δ 8.51-8.40 (m, 1H), 8.33-8.14 (m, 2H), 7.26-7.17 (m, 1H), 7.01 (t, J = 8.8 Hz, 1H), 5.37-5.16 (m, 2H), 4.74-4.66 (m, 0.54H), 4.59- 4.51 (m, 0.53H), 4.41-3.71 (m, 6H), 3.29-3.21 (m, 3H), 3.10-2.99 (m, 1H), 2.88-2.68 (m, 1H), 2.38-1.82 (m, 7H), 1.56-1.40 (m, 3H), 1.42-1.30 (m, 3H). |
| 914a | 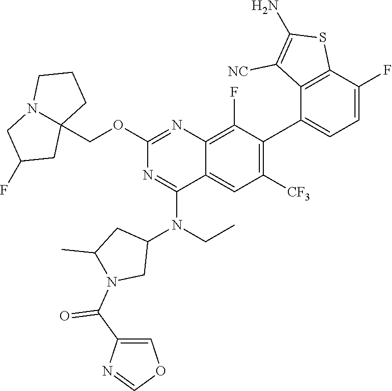 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)c2cocn2)C1] | |
| 915 | 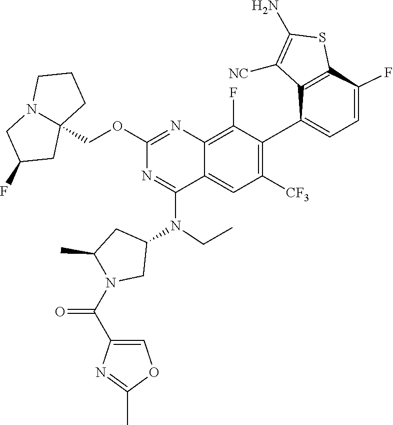 | LCMS m/z [M − 1]: 799.3 ¹H NMR (400 MHz, CD₃OD) δ 8.32-8.17 (m, 2H), 7.28-7.15 (m, 1H), 7.01 (t, J = 9.0 Hz, 1H), 5.39-5.21 (m, 2H), 4.69-4.62 (m, 0.58H), 4.50-4.43 (m, 0.59H), 4.35-3.69 (m, 6H), 3.29-3.09 (m, 3H), 3.08-2.96 (m, 1H), 2.84-2.67 (m, 1H), 2.56-2.39 (m, 3H), 2.35-1.84 (m, 7H), 1.49-1.27 (m, 6H). |
| 915a | 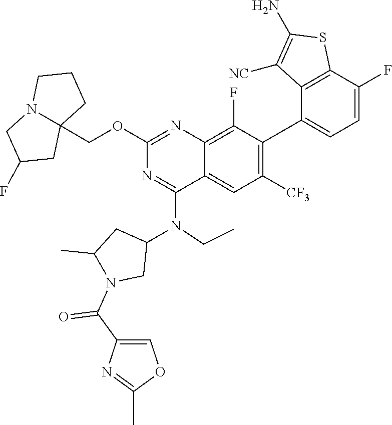 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)c2coc(C)n2)C1] | |
| 916 | 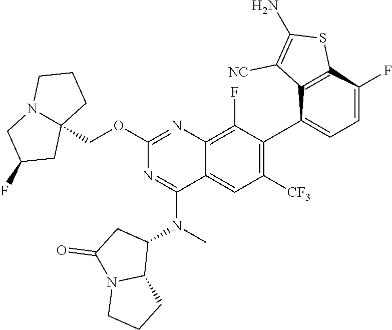 | LCMS m/z [M − 1]: 716.2 ¹H NMR (400 MHz, CD₃OD) δ 8.32-8.21 (S, 1H), 7.23- 7.19 (m, 1H), 7.03-6.98 (m, 1H), 5.37-5.32 (m, 0.5H), 5.24-5.17 (m, 1.5H), 4.20-4.15 (m, 3H), 3.84-3.79 (m, 4H), 3.25-3.17 (m, 4H), 3.03-2.89 (m, 3H), 2.25-1.89 (m, 10H). |
| 917 | 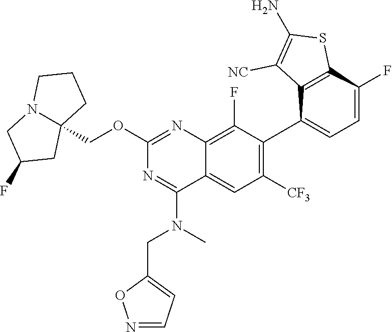 | LCMS m/z [M − 1]: 674.3 ¹H NMR (400 MHz, CD₃OD) δ 8.40-8.38 (m, 2H), 7.23- 7.20 (m, 1H), 7.01 (t, J = 8.8 Hz, 1H), 6.52 (s, 1H), 5.36- 5.15 (m, 3H), 4.24 (dd, J = 23.1, 10.6 Hz, 2H), 3.65 (s, 3H), 3.25-3.20 (m, 3H), 3.03-3.00 (m, 1H), 2.31-1.86 (m, 6H). |
| 917a | 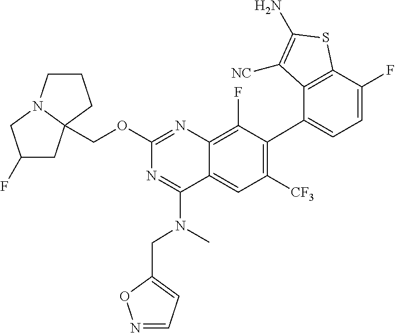 [SMILES: CN(Cc1ccno1)c1nc(OCC23CCCN2CC(F)C3)nc2c(F)c(-c3ccc(F)c4sc(N)c(C#N)c34)c(C(F)(F)F)cc12] | |
| 918 | 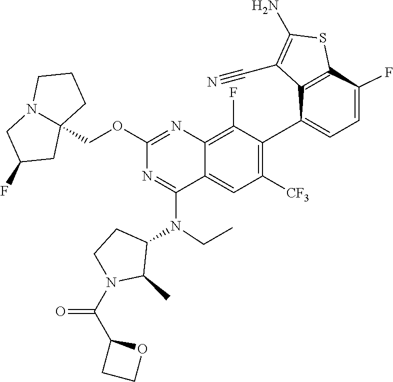 | LCMS m/z [M − 1]: 774.2 ¹H NMR (400 MHz, CD₃OD) δ 8.21 (s, 1H), 7.20-7.24 (m, 1H), 7.01 (t, 1H), 5.49-5.53 (m, 1H), 5.38 (s, 0.5H), 5.25 (s, 0.5H), 4.71-4.76 (m, 1H), 4.50-4.60 (m, 3H), 4.14-4.27 (m, 2H), 3.74-3.89 (m, 3H), 3.37-3.47 (m, 1H), 3.25-3.26 (m, 2H), 2.94-3.07 (m, 2H), 2.73-2.81 (m, 1H), 2.52-2.61 (m, 1H), 2.25-2.48 (m, 3H), 2.12- 2.22 (m, 2H), 1.88-2.02 (m, 3H), 1.29-1.47 (m, 6H). |
| 918a | 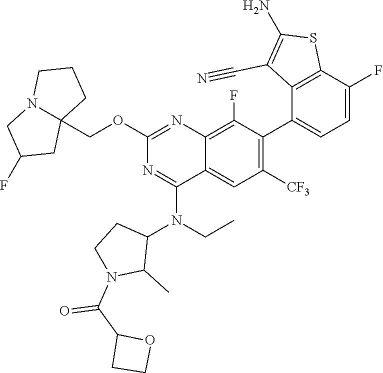 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CCO2)C1C] | |
| 919 | 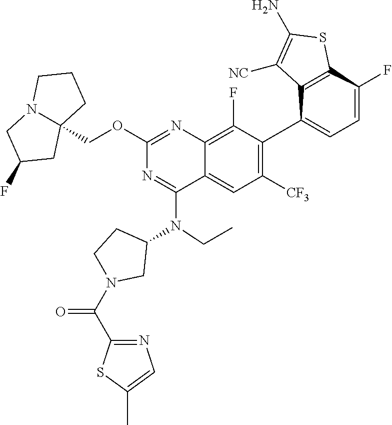 | LCMS m/z [M − 1]: 801.3 ¹H NMR (400 MHz, CD₃OD) δ 8.26 (s, 1H), 7.66 (d, J = 17.6 Hz, 1H), 7.28-7.15 (m, 1H), 7.01 (t, J = 8.9 Hz, 1H), 5.32 (d, J = 8.7 Hz, 0.5H), 5.18 (d, J = 10.2 Hz, 0.5H), 5.13-4.97 (m, 1H), 4.82-4.76 (m, 0.5H), 4.72- 4.64 (m, 0.5H), 4.29-4.01 (m, 4H), 3.97-3.83 (m, 2H), 3.73-3.60 (m, 1H), 3.28-3.09 (m, 3H), 2.98 (d, J = 5.1 Hz, 1H), 2.68-2.36 (m, 5H), 2.32-2.02 (m, 3H), 2.00-1.76 (m, 3H), 1.56-1.43 (m, 3H). |
| 919a | 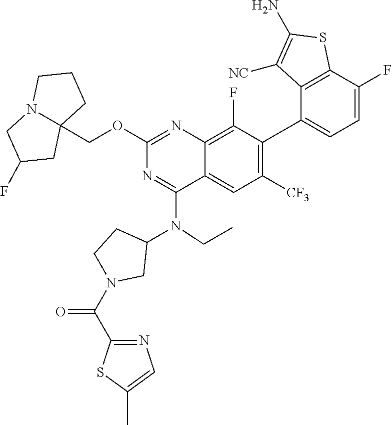 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2ncc(C)s2)C1] | |
| 920 | 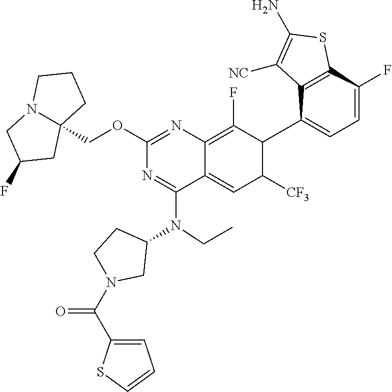 | LCMS m/z [M − 1]: 786.2 ¹H NMR (400 MHz, CD₃OD) δ 8.27 (s, 1H), 7.65-7.75 (m, 2H), 7.13-7.26 (m, 2H), 7.01 (t, 1H), 5.34 (s, 0.5H), 5.18 (s, 0.5H), 5.02-5.12 (m, 1H), 4.12-4.52 (m, 4H), 3.85-4.09 (m, 4H), 3.70 (s, 1H), 3.18-3.27 (m, 2H), 3.00 (s, 1H), 2.55-2.69 (m, 1H), 2.45 (s, 1H), 2.03-2.29 (m, 3H), 1.78-2.01 (m, 3H), 1.51 (s, 3H). |
| 920a | 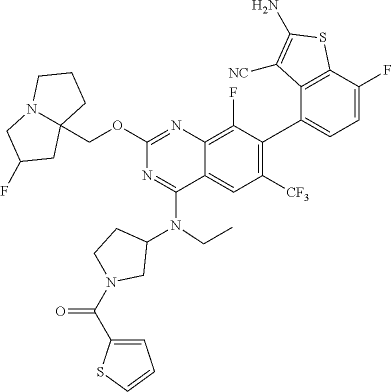 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2cccs2)C1] | |
| 921 | 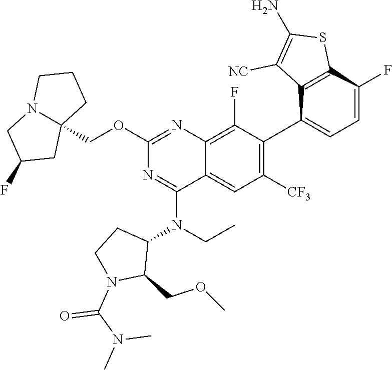 | LCMS m/z [M − 1]: 791.2 ¹H NMR (400 MHz, CD₃OD) δ 8.23 (s, 1H), 7.23-7.20 (m, 1H), 7.01 (t, 1H), 5.37 (s, 0.5H), 5.23 (s, 0.5H), 4.98-4.91 (m, 1H), 4.61-4.59 (m, 1H), 4.31-4.29 (m, 1H), 4.20-4.17 (m, 1H), 4.04-3.97 (m, 1H), 3.71-3.66 (m, 1H), 3.59-3.51 (m, 2H), 3.44-3.35 (m, 2H), 3.25- 3.16 (m, 5H), 3.04-2.97 (m, 1H), 2.87 (s, 6H), 2.66-2.55 (m, 1H), 2.39-2.28 (m, 1H), 2.25-1.94 (m, 6H), 1.91-1.82 (m, 1H), 1.45 (t, 3H). |
| 921a | 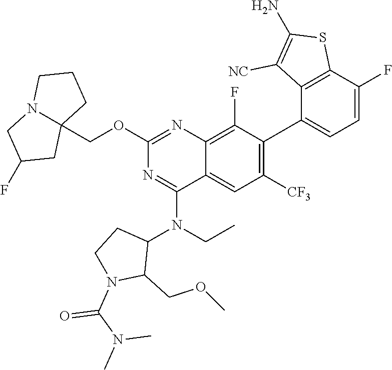 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)N(C)C)C1COC] | |
| 922 | 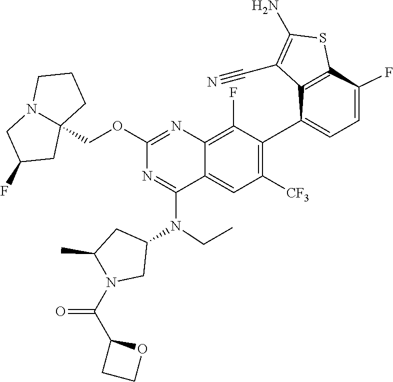 | LCMS m/z [M − 1]: 774.2 ¹H NMR (400 MHz, CD₃OD) δ 8.23 (s, 1H), 7.18-7.25 (m, 1H), 7.01 (t, 1H), 5.59 (t, 0.5H), 5.42 (s, 0.5H), 5.27-5.36 (m, 1H), 5.13-5.25 (m, 1H), 4.63-4.78 (m, 1.5H), 4.53-4.61 (m, 1H), 4.44-4.51 (m, 1H), 4.36-4.41 (m, 1H), 4.29 (d, 0.5H), 4.04-4.13 (m, 1H), 3.74-3.96 (m, 3H), 3.35-3.52 (m, 3H), 3.07-3.17 (m, 1H), 2.82- 2.97 (m, 2H), 2.53-2.79 (m, 2H), 2.26-2.45 (m, 2H), 2.13-2.24 (m, 1H), 2.02-2.11 (m, 2H), 1.89-1.99 (m, 1H), 1.38-1.50 (m, 3H), 1.22-1.35 (m, 3H). |
| 922a | 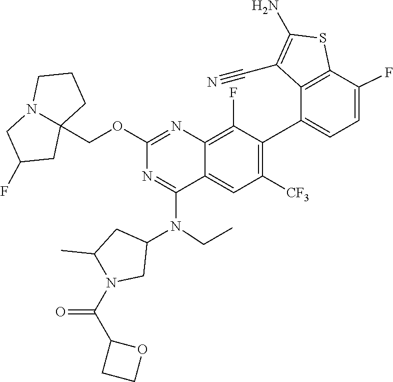 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)C2CCO2)C1] | |
| 923 | 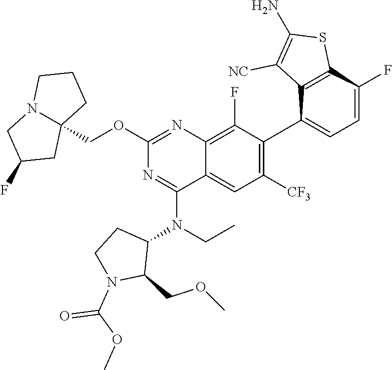 | LCMS m/z [M − 1]: 778.2 ¹H NMR (400 MHz, CD₃OD) δ 8.20 (s, 1H), 7.23-7.19 (m, 1H), 7.00 (t, 1H), 5.38 (s, 0.5H), 5.24 (s, 0.5H), 4.93-4.89 (m, 1H), 4.38-4.21 (m, 1H), 3.98-3.91 (m, 2H), 3.73-3.67 (m, 4H), 3.57-3.56 (m, 1H), 3.49-3.34 (m, 2H), 3.28-3.15 (m, 6H), 3.05-2.99 (m, 1H), 2.62- 2.53 (m, 1H), 2.37-2.25 (m, 2H), 2.21-1.97 (m, 4H), 1.93-1.85 (m, 1H), 1.46 (t, 3H). |
| 923a | 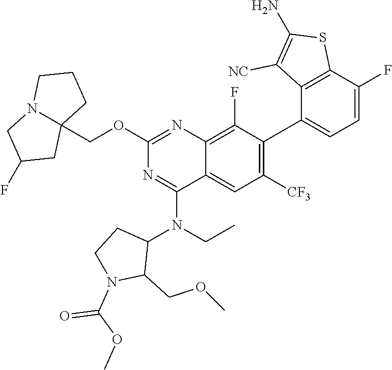 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC)C1COC] | |
| 924 | 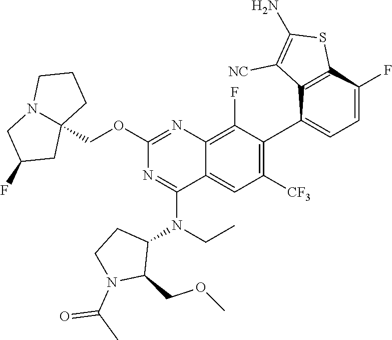 | LCMS m/z [M − 1]: 762.2 ¹H NMR (400 MHz, CD₃OD) δ 8.21 (s, 1H), 7.23-7.20 (m, 1H), 7.01 (t, 1H), 5.38 (s, 0.5H), 5.24 (s, 0.5H), 4.92-4.88 (m, 1H), 4.57-4.46 (m, 1H), 4.28-4.19 (m, 2H), 3.99-3.89 (m, 2H), 3.75-3.62 (m, 2H), 3.60-3.52 (m, 2H), 3.36-3.31 (m, 3H), 3.30-3.22 (m, 3H), 3.07- 3.00 (m, 1H), 2.67-2.56 (m, 1H), 2.45-2.34 (m, 1H), 2.33-2.19 (m, 2H), 2.18-2.10 (m, 4H), 2.04-1.96 (m, 2H), 1.94-1.86 (m, 1H), 1.48 (t, 3H). |
| 924a | 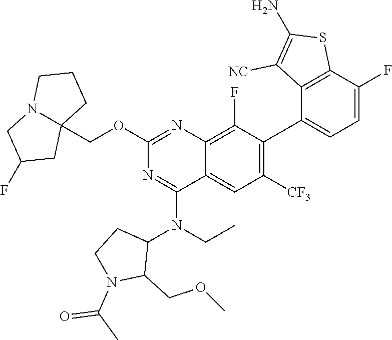 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(C)=O)C1COC] | |
| 925 | 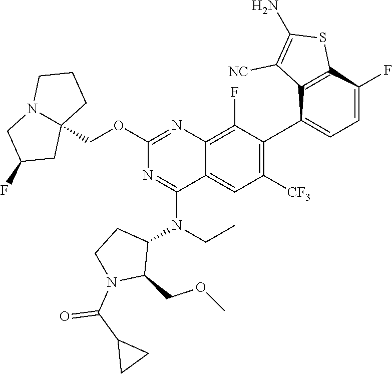 | LCMS m/z [M − 1]: 788.3 ¹H NMR (400 MHz, CD₃OD) δ 8.22 (s, 1H), 7.23-7.20 (m, 1H), 7.01 (t, 1H), 5.36 (s, 0.5H), 5.23 (s, 0.5H), 5.05-4.95 (m, 1H), 4.76-4.69 (m, 0.5H), 4.54-4.51 (m, 0.5H), 4.30-4.11 (m, 3H), 3.97-3.87 (m, 1H), 3.81-3.52 (m, 4H), 3.37-3.35 (m, 3H), 3.25-3.21 (m, 3H), 3.04- 2.96 (m, 1H), 2.66-2.56 (m, 1H), 2.53-2.31 (m, 2H), 2.30-2.05 (m, 3H), 2.01-1.93 (m, 2H), 1.91-1.88 (m, 1H), 1.51-1.41 (m, 3H), 0.95-0.79 (m, 4H). |
| 925a | 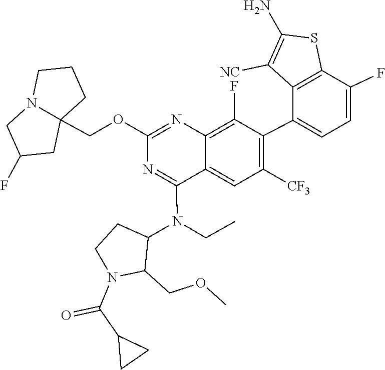 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)C2CC2)C1COC] | |
| 926 | 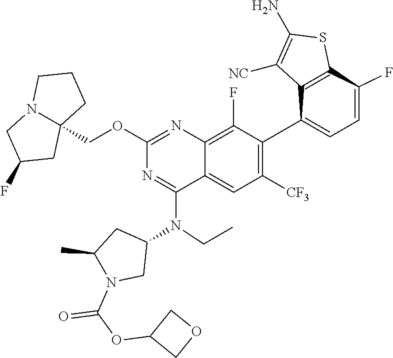 | LCMS m/z [M − 1]: 790.2 ¹H NMR (400 MHz, CD₃OD) δ 8.23 (s, 1H), 7.22 (dd, J = 8.2, 5.1 Hz, 1H), 7.01 (t, J = 8.9 Hz, 1H), 5.47-5.35 (m, 1.5H), 5.25-5.16 (m, 1.5H), 4.90 (s, 1H), 4.64 (d, J = 5.6 Hz, 2H), 4.26 (dt, J = 29.9, 15.0 Hz, 3H), 4.03- 3.95 (m, 0.5H), 3.92-3.78 (m, 2.5H), 3.72-3.59 (m, 1H), 3.30 (s, 2H), 3.26-3.18 (m, 2H), 3.05-2.96 (m, 1H), 2.65 (s, 1H), 2.35-1.86 (m, 7H), 1.46-1.29 (m, 6H). |
| 926a | 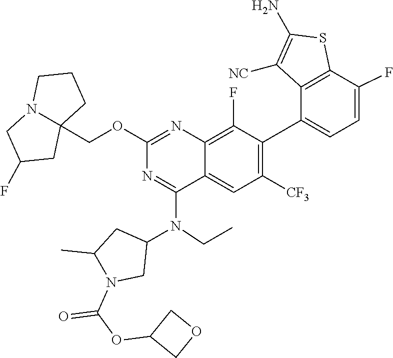 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)OC2COC2)C1] | |
| 927 | 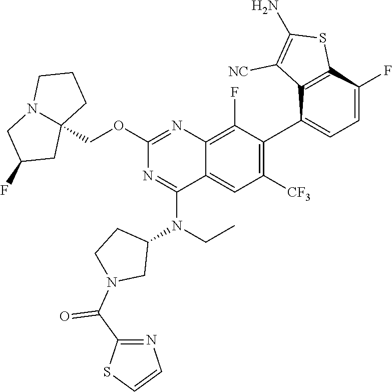 | LCMS m/z [M − 1]: 787.1 ¹H NMR (400 MHz, CD₃OD) δ 8.27 (s, 1H), 8.02 (d, J = 3.1 Hz, 0.5H), 7.98 (d, J = 3.1 Hz, 0.5H), 7.84 (d, J = 3.1 Hz, 1H), 7.21 (T, J = 5.1 Hz, 1H), 7.01 (t, J = 8.9 Hz, 1H), 5.29 (s, 0.5H), 5.15 (s, 0.5H), 5.12-5.00 (m, 1H), 4.33-4.03 (m, J = 43.0, 12.3 Hz, 5H), 3.98-3.83 (m, J = 14.2, 7.0 Hz, 3H), 3.78-3.51 (m, 1H), 3.15 (d, J = 15.3 Hz, 2H), 2.95 (s, 1H), 2.66-2.42 (m, 2H), 2.20 (t, J = 26.1 Hz, 2H), 2.04 (d, J = 9.6 Hz, 1H), 1.91 (s, 2H), 1.80 (s, 1H), 1.52 (s, 3H). |
| 927a | 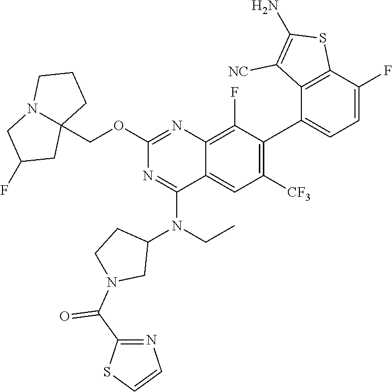 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2nccs2)C1] | |
| 928 | 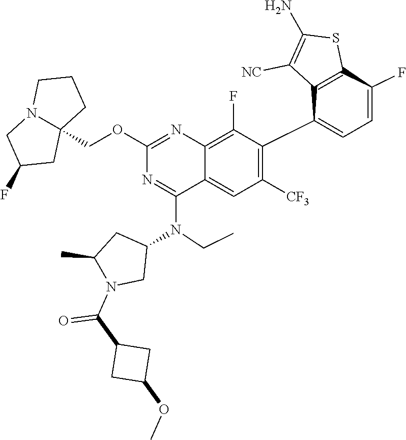 | LCMS m/z [M − 1]: 802.2 ¹H NMR (400 MHz, CD₃OD) δ 8.24 (s, 1H), 7.24-7.21 (m, 1H), 7.04-7.00 (m, 1H), 5.40-5.18 (m, 2H), 4.44- 4.21 (m, 3H), 3.98-3.64 (m, 5H), 3.32-3.30 (m, 2H), 3.25-3.23 (m, 4H), 3.09-3.03 (m, 1H), 3.03-2.79 (m, 1H), 2.68-2.60 (m, 1H), 2.49-2.38 (m, 2H), 2.29-1.88 (m, 9H), 1.46-1.40 (m, 3H), 1.31-1.23 (m, 3H). |
| 928a | 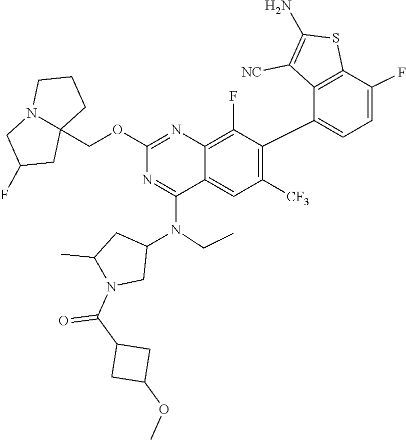 [SMILES: CCN(c1nc(OCC23CCCN2CC(F)C3)nc2c(F)c(-c3ccc(F)c4sc(N)c(C#N)c34)c(C(F)(F)F)cc12)C1CC(C)N(C(=O)C2CC(OC)C2)C1] | |
| 929 | 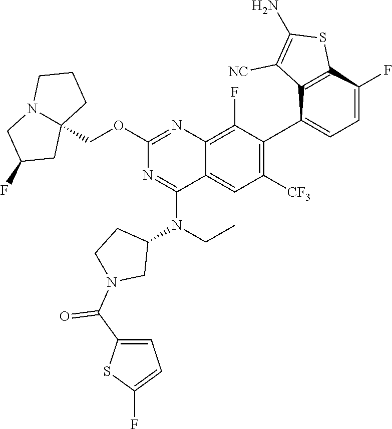 | LCMS m/z [M − 1]: 820.1 ¹H NMR (400 MHz, CD₃OD) δ 8.28 (s, 1H), 7.53-7.49 (s, 1H), 7.24-7.21 (m, 1H), 7.11-7.00 (m, 2H), 5.32 (s, 0.5H), 5.19 (s, 0.5H), 5.05 (s, 1H), 4.31-4.16 (m, 3H), 3.90-3.86 (m, 3H), 3.64-3.48 (m, 2H), 3.21-3.15 (m, 3H), 3.00 (s, 1H), 2.75-1.83 (m, 8H), 1.52 (s, 3H). |
| 929a | 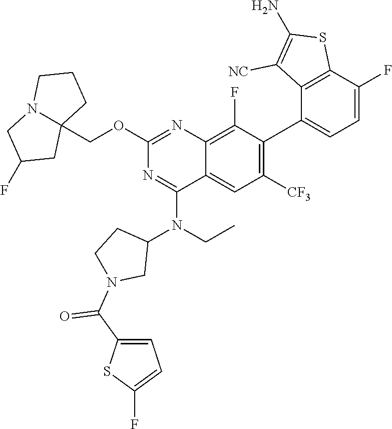 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2ccc(F)s2)C1] | |
| 930 | 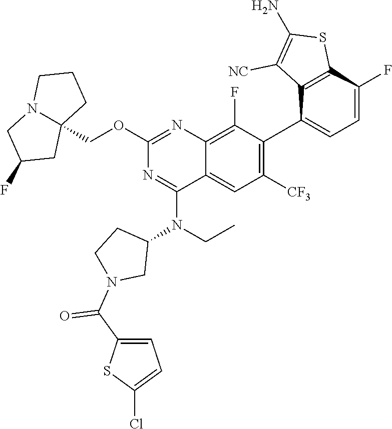 | LCMS m/z [M − 1]: 804.1 ¹H NMR (400 MHz, CD₃OD) δ 8.27 (s, 1H), 7.41 (s, 1H), 7.24-7.19 (m, 1H), 7.04-6.99 (m, 1H), 6.65 (s, 1H), 5.32 (s, 0.5H), 5.18 (s, 0.5H), 5.05 (m, 1H), 4.58-4.42 (m, 1H), 4.26-4.14 (m, 2H), 4.03-3.59 (m, 5H), 3.22- 3.13 (m, 3H), 2.99 (m, 1H), 2.63-1.82 (m, 8H), 1.51 (s, 3H). |
| 930a | 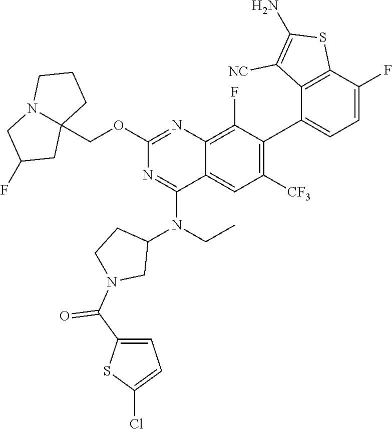 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2ccc(Cl)s2)C1] | |
| 931 | 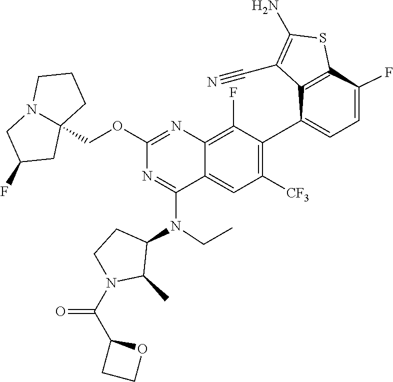 | LCMS m/z [M − 1]: 774.2 ¹H NMR (400 MHz, CD₃OD) δ 8.29 (d, 1H), 7.25-7.19 (m, 1H), 7.01 (t, 1H), 5.49-5.31 (m, 1.5H), 5.26-5.20 (m, 0.5H), 4.78-4.56 (m, 3H), 4.37-4.24 (m, 2H), 4.08- 3.99 (m, 1H), 3.95-3.75 (m, 2H), 3.72-3.53 (m, 2H), 3.27-3.22 (m, 2H), 3.04-2.95 (m, 2H), 2.85-2.70 (m, 1H), 2.64-2.52 (m, 1H), 2.50-2.44 (m, 1H), 2.40-2.17 (m, 3H), 2.05-1.83 (m, 4H), 1.41-1.33 (m, 3H), 1.09- 0.93 (m, 3H). |
| 932 | 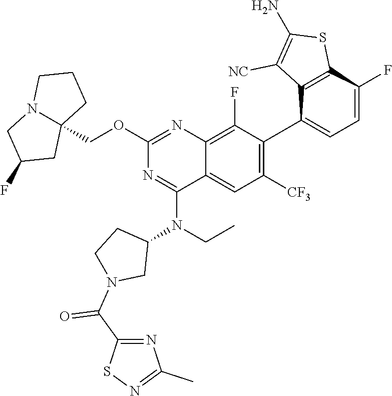 | LCMS m/z [M − 1]: 802.4 ¹H NMR (400 MHz, CD₃OD) δ = 8.27 (s, 1H), 7.21 (d, J = 4.8 Hz, 1H), 7.02 (t, J = 9.2 Hz, 1H), 5.30 (d, J = 53.2 Hz, 1H), 5.12-5.04 (m, 1H), 4.75-4.70 (m, 1H), 4.31- 4.23 (m, 3H), 4.11 (s, 1H), 3.93-3.92 (m, 2.5H), 3.73 (s, 0.5H), 3.27-3.20 (m, 3H), 3.05 (s, 1H), 2.70 (d, J = 23.2 Hz, 3H), 3.27-3.18 (m, 4H), 2.15-2.11 (m, 1H), 2.05-1.94 (m, 2H), 1.92-1.80 (m, 1H), 1.55- 1.49 (m, 3H). |
| 932a | 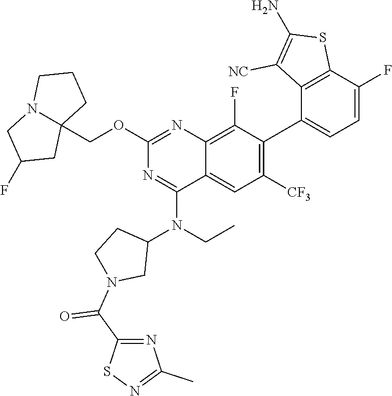 | |
| 933 | 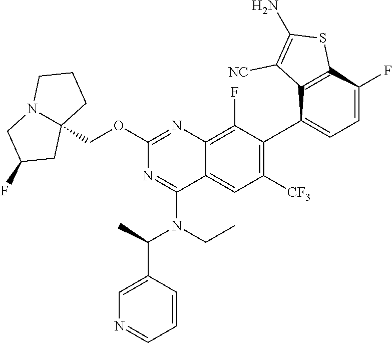 | LCMS m/z [M − 1]: 712.1 ¹H NMR (400 MHz, CD₃OD) δ 8.95 (s, 1H), 8.45 (d, 1H), 8.09-8.27 (m, 1H), 7.61 (d, 1H), 7.22-7.25 (m, 1H), 7.04 (t, 1H), 5.72-5.74 (m, 1H), 5.65 (s, 0.5H), 5.52 (s, 0.5H), 4.81 (d, 1H), 4.72 (d, 1H), 3.87-4.06 (m, 5H), 3.47-3.562 (m, 1H), 2.61-2.78 (m, 2H), 2.42-2.51 (m, 1H), 2.34-2.38 (m, 2H), 2.13-2.24 (m, 1H), 1.64 (d, 1H), 1.51-1.59 (m, 6H). |
| 933a | 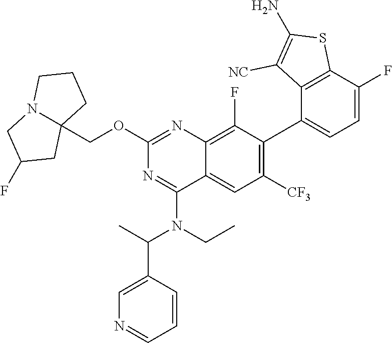 | |
| 934 | 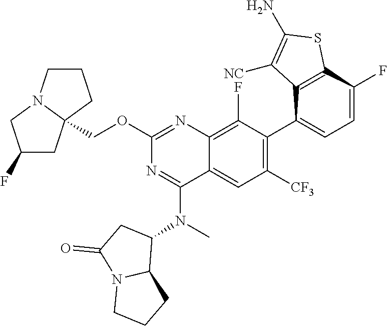 | LCMS m/z [M − 1]: 716.2 ¹H NMR (400 MHz, CD₃OD) δ 8.32-8.21 (S, 1H), 7.23- 7.19 (m, 1H), 7.03-6.98 (m, 1H), 5.36-5.27 (m, 0.5H), 5.26-5.17 (m, 1.5H), 4.25-4.16 (m, 3H), 3.83-3.31 (m, 4H), 3.30-3.05 (m, 5H), 3.05-2.88 (m, 2H), 2.20-2.11 (m, 5H), 2.11-1.70 (m, 5H). |
| 935 | 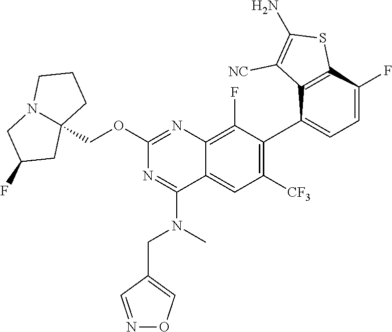 | LCMS m/z [M − 1]: 674.2 ¹H NMR (400 MHz, CD₃OD): δ 8.85 (s, 1 H), 8.60 (s, 1 H), 8.36 (s, 1 H), 7.18-7.23 (m, 1 H), 6.98-7.04 (m, 1 H), 5.42 (s, 0.5 H), 5.29 (s, 0.5 H), 4.89-5.01 (m, 2 H), 4.29-4.45 (m, 2 H), 3.56 (s, 3 H), 3.36-3.45 (m, 2 H), 3.31-3.32 (m, 1 H), 3.08-3.16 (m, 1 H), 1.90-2.38 (m, 6 H). |
| 935a | 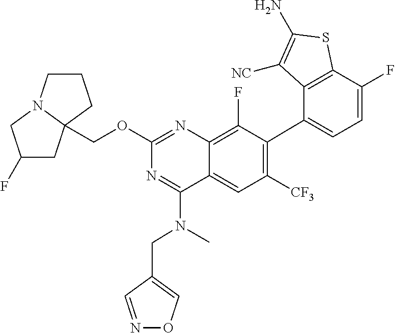 [SMILES: CN(Cc1cnoc1)c1nc(OCC23CCCN2CC(F)C3)nc2c(F)c(-c3ccc(F)c4sc(N)c(C#N)c34)c(C(F)(F)F)cc12] | |
| 936 | 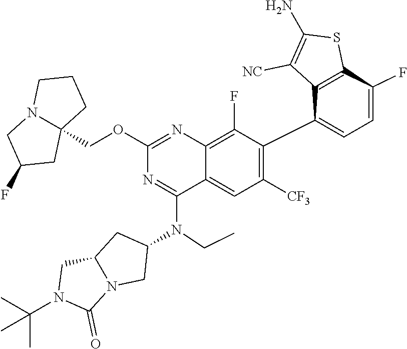 | LCMS m/z [M − 1]: 787.2 ¹H NMR (400 MHz, CD₃OD) δ 8.20 (s, 1H), 7.21 (dd, J = 8.3, 5.0 Hz, 1H), 7.01 (dd, J = 9.4, 8.5 Hz, 1H), 5.37 (s, 0.5H), 5.34-5.25 (m, J = 15.1, 7.9 Hz, 1H), 5.24 (s, 0.5H), 4.34 (d, J = 10.5 Hz, 1H), 4.24 (d, J = 10.5 Hz, 1H), 3.86-3.74 (m, J = 12.4, 6.3 Hz, 2H), 3.72-3.63 (m, J = 10.0, 7.7 Hz, 2H), 3.60-3.46 (m, 3H), 3.29- 3.20 (m, J = 19.9 Hz, 3H), 3.06-2.99 (m, J = 9.5, 5.7 Hz, 1H), 2.39-2.28 (m, 2H), 2.26-2.21 (m, 1H), 2.19- 2.13 (m, 1H), 2.04-1.97 (m, J = 10.9, 6.3 Hz, 2H), 1.94-1.86 (m, 2H), 1.42 (s, 9H), 1.36 (t, J = 6.9 Hz, 3H). |
| 936a | 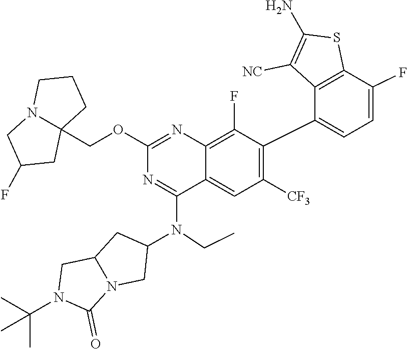 [SMILES: CCN(c1nc(OCC23CCCN2CC(F)C3)nc2c(F)c(-c3ccc(F)c4sc(N)c(C#N)c34)c(C(F)(F)F)cc12)C1CC2CN(C(C)(C)C)C(=O)N2C1] | |
| 937 | 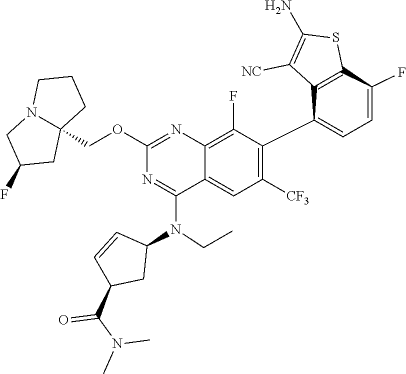 | LCMS m/z [M − 1]: 744.2 ¹H NMR (400 MHz, CD₃OD) δ = 8.29 (s, 1H), 7.22 (dd, J = 8.3, 5.0, 1H), 7.01 (dd, J = 9.4, 8.5, 1H), 6.08-5.97 (m, 2H), 5.79 (s, 1H), 5.42 (s, 0.5H), 5.29 (s, 0.5H), 4.42 (d, J = 10.9, 1H), 4.30 (d, J = 10.9, 1H), 4.03 (d, J = 7.2, 1H), 3.85 (dt, J = 22.9, 7.3, 2H), 3.53-3.32 (m, 3H), 3.21 (d, J = 10.5, 3H), 3.10 (d, J = 5.9, 1H), 3.02- 2.85 (m, 4H), 2.47-2.28 (m, 2H), 2.19 (dt, J = 12.8, 6.3, 2H), 2.09-2.02 (m, 2H), 1.92 (s, 1H), 1.40 (t, J = 6.9, 3H). |
| 937a | 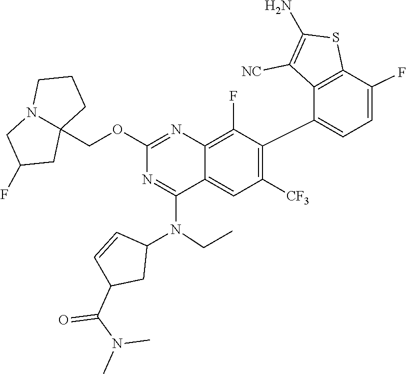 [SMILES: CCN(c1nc(OCC23CCCN2CC(F)C3)nc2c(F)c(-c3ccc(F)c4sc(N)c(C#N)c34)c(C(F)(F)F)cc12)C1C=CC(C(=O)N(C)C)C1] | |
| 938 | 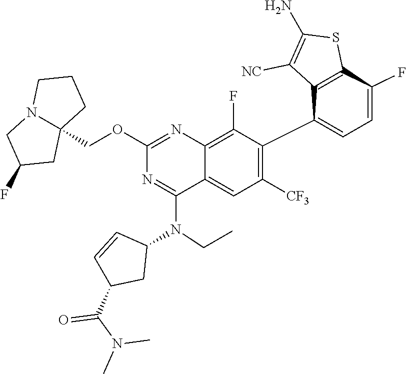 | LCMS m/z [M − 1]: 744.2 ¹H NMR (400 MHz, CD₃OD) δ 8.27 (s, 1H), 7.24-7.19 (m, 1H), 7.03-6.98 (m, 1H), 6.04-5.96 (m, 2H), 5.78- 5.72 (m, 1H), 5.39 (s, 0.5H), 5.25 (s, 0.5H), 4.37-4.26 (m, 2H), 4.06-4.01 (m, 1H), 3.93-3.85 (m, 1H), 3.81- 3.74 (m, 1H), 3.29-3.25 (m, 3H), 3.19 (s, 3H), 3.08-3.02 (m, 1H), 2.98 (s, 3H), 2.35-2.18 (m, 4H), 2.05-1.91 (m, 4H), 1.41-1.36 (m, 3H). |
| 939 | 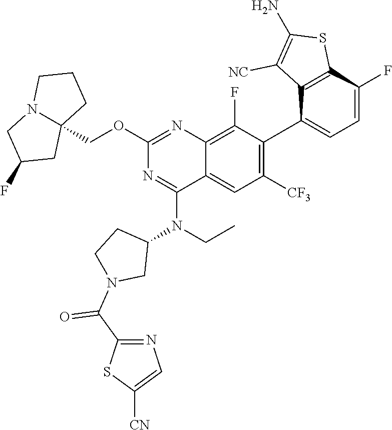 | LCMS m/z [M − 1]: 821.4 ¹H NMR (400 MHz, CD₃OD) δ 8.27 (s, 1H), 7.90-7.86 (m, 1H), 7.24-7.19 (m, 1H), 7.04-7.00 (m, 1H), 5.34 (s, 0.5H), 5.20 (s, 0.5H), 5.11-4.98 (m, 1H), 4.84-4.77 (m, 0.6H), 4.73-4.65 (m, 0.5H), 4.31-4.17 (m, 3H), 4.12- 4.02 (m, 1H), 3.99-3.83 (m, 2H), 3.73-3.65 (m, 1H), 3.27-3.11 (m, 3H), 3.05-2.97 (m, 1H), 2.66-2.38 (m, 2H), 2.34-2.02 (m, 3H), 1.99-1.69 (m, 3H), 1.53-1.49 (m, 3H). |
| 939a | 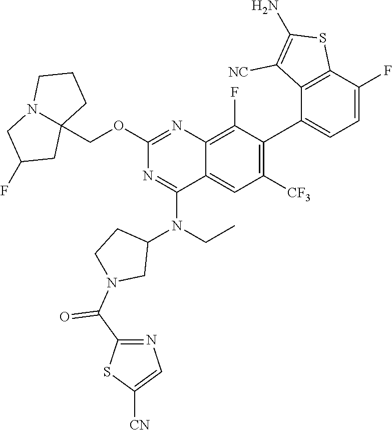 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2ncc(C#N)s2)C1] | |
| 940 | 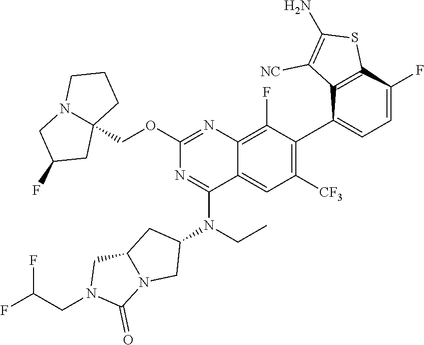 | LCMS m/z [M − 1]: 795.2 ¹H NMR (400 MHz, CD₃OD) δ 8.21 (s, 1H), 7.26- 7.16 (m, 1H), 7.04-6.92 (m, 1H), 6.03 (t, J = 55.4 Hz, 1H), 5.46-5.12 (m, 2H), 4.41-4.20 (m, 2H), 4.00- 3.90 (m, 1H), 3.86-3.73 (m, 2H), 3.73-3.52 (m, 6H), 3.11-2.99 (m, 1H), 2.44-2.24 (m, 3H), 2.23-2.15 (m, 1H), 2.08-1.98 (m, 3H), 1.97-1.86 (m, 1H), 1.38 (t, J = 7.0 Hz, 3H). |
| 940a | 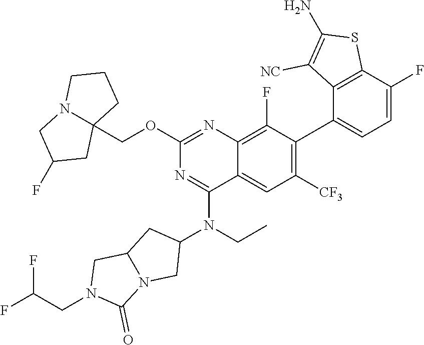 [SMILES: CCN(c1nc(OCC23CCCN2CC(F)C3)nc2c(F)c(-c3ccc(F)c4sc(N)c(C#N)c34)c(C(F)(F)F)cc12)C1CC2CN(CC(F)F)C(=O)N2C1] | |
| 941 | 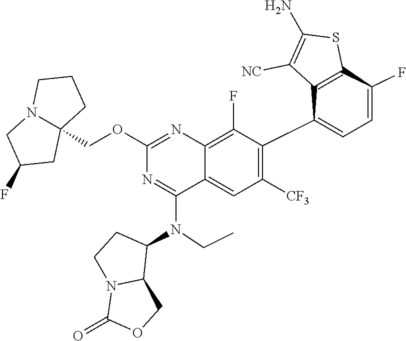 | LCMS m/z [M − 1]: 732.2 ¹H NMR (400 MHz, CD₃OD) δ 8.21 (s, 1H), 7.25- 7.15 (m, 1H), 7.06-6.96 (m, 1H), 5.51 (s, 0.5H), 5.37 (s, 0.5H), 4.73 (s, 1H), 4.61-4.35 (m, 4H), 4.10-3.98 (m, 2H), 3.97-3.49 (m, 5H), 3.28-3.19 (m, 2H), 2.72- 2.53 (m, 2H), 2.51-2.26 (m, 3H), 2.22-2.11 (m, 2H), 2.03 (s, 1H), 1.51 (t, J = 6.8 Hz, 3H). |
| 941a | 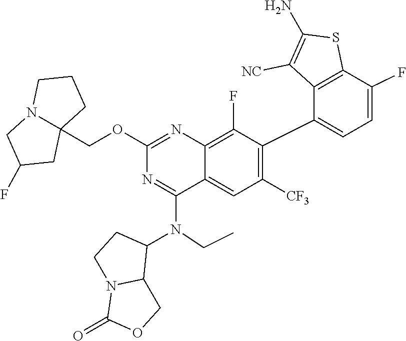 [SMILES: CCN(c1nc(OCC23CCCN2CC(F)C3)nc2c(F)c(-c3ccc(F)c4sc(N)c(C#N)c34)c(C(F)(F)F)cc12)C1CCN2C(=O)OCC12] | |
| 942 | 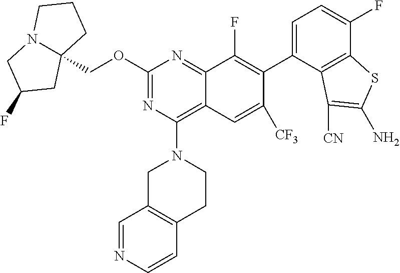 [SMILES: N#Cc1c(N)sc2c(F)ccc(-c3c(C(F)(F)F)cc4c(N5CCc6ccncc6C5)nc(OC[C@@]56CCCN5C[C@H](F)C6)nc4c3F)c12] | LCMS m/z [M − 1]: 696.3 ¹H NMR (400 MHz, CD₃OD) δ 8.83 (s, 1H), 8.60-8.65 (m, 1H), 8.34 (s, 1H), 7.90-7.93 (m, 1H), 7.22-7.26 (m, 1H), 7.04 (t, J = 9.0 Hz, 1H), 5.57 (d, J = 51.2 Hz, 1H), 5.30-5.40 (m, 2H), 4.66-4.81 (m, 2H), 4.28-4.38 (m, 2H), 3.85-4.07 (m, 3H), 3.35 (s, 3H), 2.16-2.76 (m, 6H). |
| 942a | 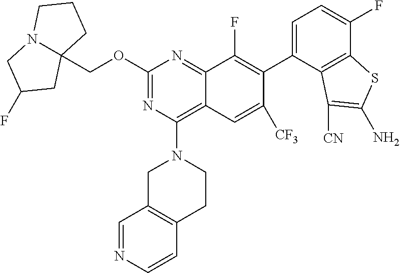 [SMILES: N#Cc1c(N)sc2c(F)ccc(-c3c(C(F)(F)F)cc4c(N5CCc6ccncc6C5)nc(OCC56CCCN5CC(F)C6)nc4c3F)c12] | |
| 943 | 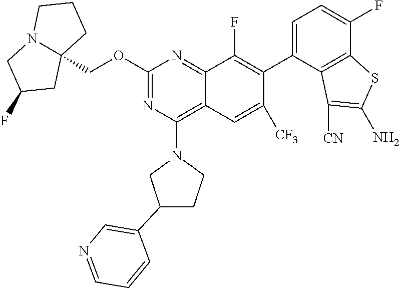 [SMILES: N#Cc1c(N)sc2c(F)ccc(-c3c(C(F)(F)F)cc4c(N5CCC(c6cccnc6)C5)nc(OC[C@@]56CCCN5C[C@H](F)C6)nc4c3F)c12] | LCMS m/z [M − 1]: 710.3 ¹H NMR (400 MHz, CD₃OD) δ 8.88 (s, 1H), 8.74 (s, 1H), 8.47-8.56 (m, 2H), 7.92-7.98 (m, 1H), 7.18-7.24 (m, 1H), 7.03 (t, J = 8.9 Hz, 1H), 5.55 (d, J = 51.4 Hz, 1H), 4.80-4.55 (m, 3H), 4.24-4.42 (m, 2H), 4.10-4.23 (m, 1H), 3.84-3.99 (m, 4 H), 3.40-3.50 (m, 1H), 2.56- 2.71 (m, 3H), 2.29-2.46 (m, 4H), 2.07-2.20 (m, 1H). |
| 943a | 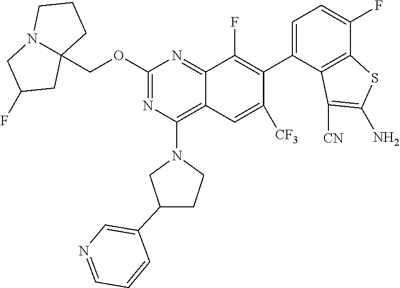 [SMILES: N#Cc1c(N)sc2c(F)ccc(-c3c(C(F)(F)F)cc4c(N5CCC(c6cccnc6)C5)nc(OCC56CCCN5CC(F)C6)nc4c3F)c12] | |
| 944 | 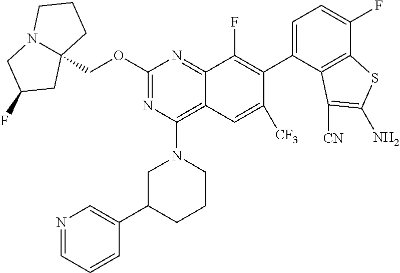 [SMILES: N#Cc1c(N)sc2c(F)ccc(-c3c(C(F)(F)F)cc4c(N5CCCC(c6cccnc6)C5)nc(OC[C@@]56CCCN5C[C@H](F)C6)nc4c3F)c12] | LCMS m/z [M − 1]: 724.3 ¹H NMR (400 MHz, CD₃OD) δ 8.85 (s, 1H), 8.70-8.74 (m, 1H), 8.52-8.58 (m, 1H), 8.15-8.19 (m, 1H), 7.92- 7.98 (m, 1H), 7.21-7.26 (m, 1H), 7.03 (t, J = 8.9 Hz, 1H), 5.55 (d, J = 51.4 Hz, 1H), 4.55-4.75 (m, 4H), 3.82- 4.08 (m, 3H), 3.42-3.68 (m, 4H), 2.54-2.79 (m, 2H), 2.25-2.48 (m, 4H), 1.89-2.18 (m, 4H). |
| 944a | 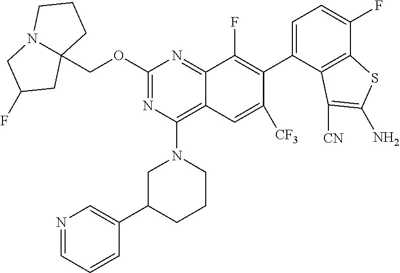 [SMILES: N#Cc1c(N)sc2c(F)ccc(-c3c(C(F)(F)F)cc4c(N5CCCC(c6cccnc6)C5)nc(OCC56CCCN5CC(F)C6)nc4c3F)c12] | |
| 945 | 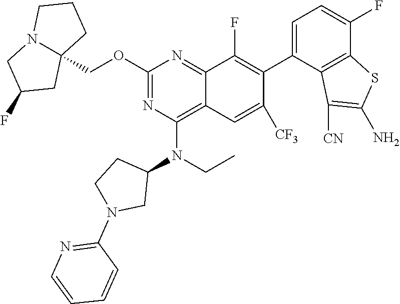 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@@H]1CCN(c2ccccn2)C1] | LCMS m/z [M − 1]: 753.4 ¹H NMR (400 MHz, CD₃OD) δ 8.33 (s, 1H), 8.15- 7.99 (m, 1H), 7.96 (d, J = 6.2 Hz, 1H), 7.32-7.20 (m, 1H), 7.18 (d, J = 9.4 Hz, 1H), 7.14-6.85 (m, 2H), 5.61- 5.46 (m, 1H), 5.34-5.22 (m, 1H), 4.79-4.42 (m, 2H), 4.16 (dd, J = 21.9, 11.5 Hz, 1H), 4.12-3.53 (m, 7H), 3.73 (dd, J = 18.3, 8.5 Hz, 1H), 3.44 (s, 1H), 2.87- 2.39 (m, 4H), 2.47-2.14 (m, 3H), 2.03 (s, 1H), 1.49 (dt, J = 60.4, 30.1 Hz, 3H). |
| 945a | 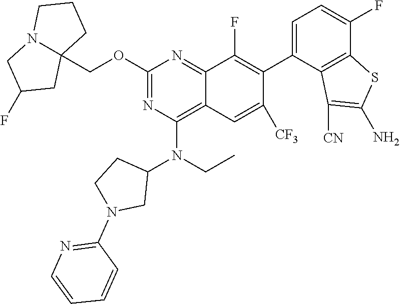 [SMILES: CCN(c1nc(OCC23CCCN2CC(F)C3)nc2c(F)c(-c3ccc(F)c4sc(N)c(C#N)c34)c(C(F)(F)F)cc12)C1CCN(c2ccccn2)C1] | |
| 946 | 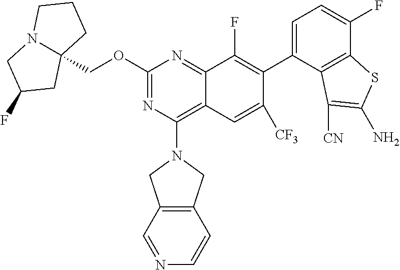 [SMILES: N#Cc1c(N)sc2c(F)ccc(-c3c(C(F)(F)F)cc4c(N5Cc6ccncc6C5)nc(OC[C@@]56CCCN5C[C@H](F)C6)nc4c3F)c12] | LCMS m/z [M − 1]: 682.3 ¹H NMR (400 MHz, CD₃OD) δ 8.89 (s, 1H), 8.67-8.72 (m, 2H), 7.90-7.93 (m, 1H), 7.22-7.26 (m, 1H), 7.04 (t, J = 9.0 Hz, 1H), 5.55-5.67 (m, 5H), 4.66-4.85 (m, 2H), 3.88-4.11 (m, 3H), 3.45-3.49 (m, 1H), 2.16-2.76 (m, 6H). |
| 946a | 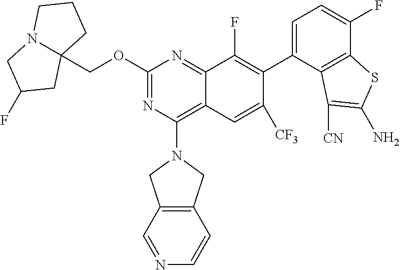 [SMILES: N#Cc1c(N)sc2c(F)ccc(-c3c(C(F)(F)F)cc4c(N5Cc6ccncc6C5)nc(OCC56CCCN5CC(F)C6)nc4c3F)c12] | |
| 947 | 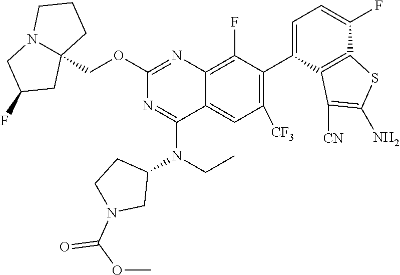 | LCMS m/z [M − 1]: 734.3 ¹H NMR (400 MHz, CD₃Cl): δ 8.29 (s, 1H), 7.21-7.25 (m, 1H), 7.03 (t, J = 8.86 Hz, 1H), 5.56 (d, J = 51.60 Hz, 1H), 5.02-5.08 (m, 1H), 4.66 (S, 2H), 3.43-4.04 (m, 13H), 2.16-2.76 (m, 8H), 1.48 (t, J = 6.96 Hz, 3H). |
| 947a | 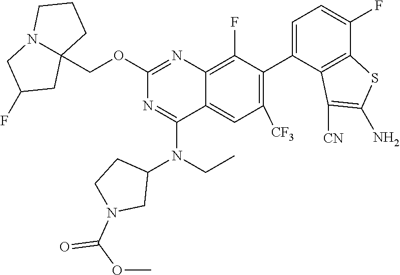 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)OC)C1] | |
| 948 | 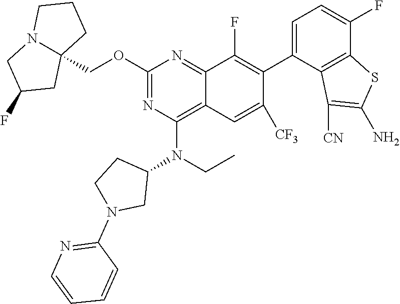 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@H]1CCN(c2ccccn2)C1] | LCMS m/z [M − 1]: 753.4 ¹H NMR (400 MHz, CD₃OD) δ 8.33 (s, 1H), 8.02- 8.07 (m, 1H), 7.96 (d, J = 6.3 Hz, 1H), 7.17-7.25 (m, 2H), 6.97-7.06 (m, 2H), 5.52 (d, J = 52.3 Hz, 1H), 5.28-5.34 (m, 1H), 4.61-4.73 (m, 2H), 4.3-4.21 (m, 1H), 3.81-4.03 (m, 7H), 3.69-3.77 (m, 1H), 3.42- 3.49 (m, 1H), 2.53-2.71 (m, 4H), 2.26-2.39 (m, 3H), 2.08-2.18 (m, 1H), 1.49-1.55 (m, 3H). |
| 948a | 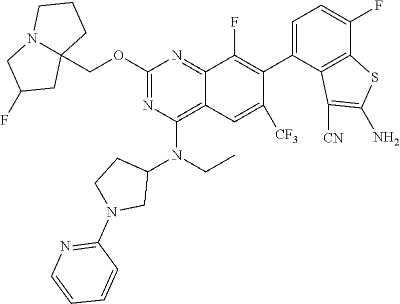 [SMILES: CCN(c1nc(OCC23CCCN2CC(F)C3)nc2c(F)c(-c3ccc(F)c4sc(N)c(C#N)c34)c(C(F)(F)F)cc12)C1CCN(c2ccccn2)C1] | |
| 949 | 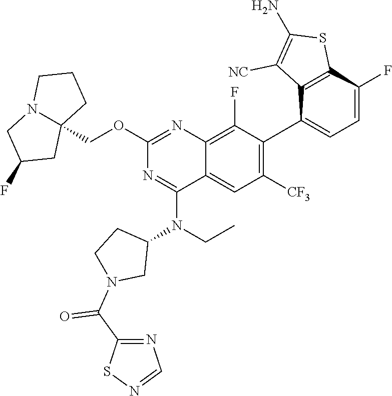 | LCMS m/z [M − 1]: 788.3 ¹H NMR (400 MHz, CD₃OD) δ 9.00-8.83 (m, 1H), 8.58-8.44 (brs, 1H), 8.27 (s, 1H), 7.27-7.15 (m, 1H), 7.01 (t, J = 8.8 Hz, 1H), 5.39-5.24 (m, 1H), 5.15-5.03 (m, 1H), 4.82-4.64 (m, 1H), 4.46-3.66 (m, 7H), 3.47- 3.31 (m, 3H), 3.14-2.99 (m, 1H), 2.68-1.94 (m, 7H), 1.93-1.80 (m, 1H), 1.65-1.34 (m, 3H). |
| 949a | 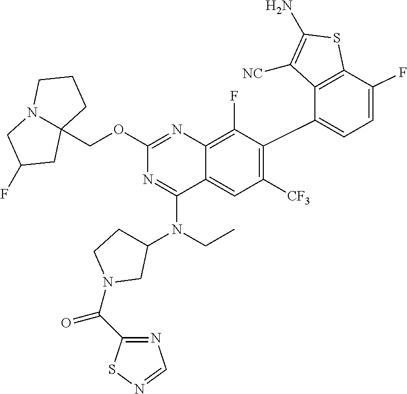 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2ncns2)C1] | |
| 950 | 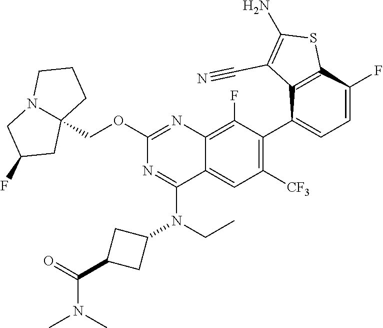 | LCMS m/z [M − 1]: 732.2 ¹H NMR (400 MHz, CD₃OD) δ 8.21-8.17 (S, 1H), 7.22- 7.19 (m, 1H), 7.03-6.98 (m, 1H), 5.37-5.32 (S, 0.5H), 5.23 (S, 0.5H), 4.90 (S, 1H), 4.30-4.22 (m, 2H), 3.97- 3.83 (m, 2H), 3.37-3.31 (m, 2H), 3.31-3.17 (m, 3H), 2.97-2.95 (m, 6H), 2.68-2.60 (m, 4H), 2.24-2.13 (m, 3H), 2.13-1.88 (m, 3H), 1.40-1.28 (m, 3H). |
| 950a | 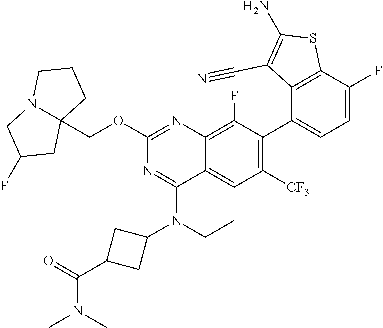 [SMILES: CCN(c1nc(OCC23CCCN2CC(F)C3)nc2c(F)c(-c3ccc(F)c4sc(N)c(C#N)c34)c(C(F)(F)F)cc12)C1CC(C(=O)N(C)C)C1] | |
| 951 | 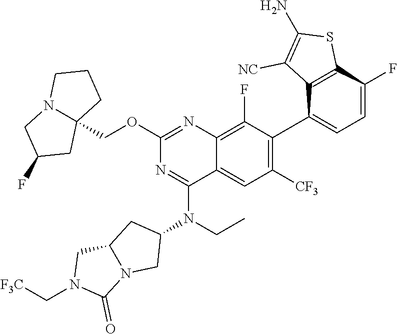 | LCMS m/z [M − 1]: 813.2 ¹H NMR (400 MHz, CD₃OD) δ 8.20 (s, 1H), 7.17-7.24 (m, 1H), 7.01 (t, 1H), 5.37 (s, 0.5H), 5.26-5.35 (m, 1H), 5.25 (s, 0.5H), 4.34 (d, 1H), 4.24 (d, 1H), 3.88-4.08 (m, 3H), 3.75-3.86 (m, 2H), 3.67-3.74 (m, 1H), 3.53-3.65 (m, 3H), 3.19-3.29 (m, 3H), 2.99-3.08 (m, 1H), 2.37- 2.46 (m, 1H), 2.27-2.36 (m, 1H), 2.22-2.26 (m, 1H), 2.13-2.21 (m, 1H), 1.86-2.05 (m, 4H), 1.37 (t, 3H). |
| 951a | 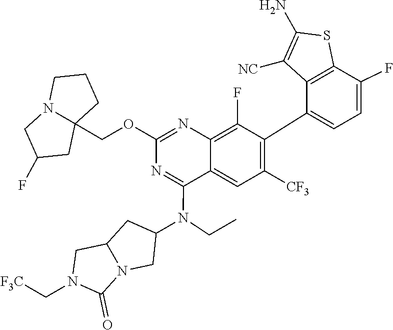 [SMILES: CCN(c1nc(OCC23CCCN2CC(F)C3)nc2c(F)c(-c3ccc(F)c4sc(N)c(C#N)c34)c(C(F)(F)F)cc12)C1CC2CN(CC(F)(F)F)C(=O)N2C1] | |
| 952 | 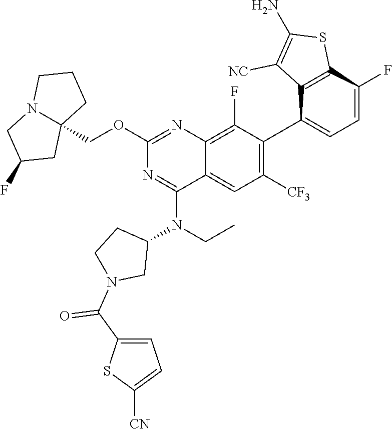 | LCMS m/z [M − 1]: 811.1 ¹H NMR (400 MHz, CD₃OD) δ 8.26 (s, 1H), 7.68-7.80 (m, 2H), 7.20-7.23 (m, 1H), 7.01 (t, 1H), 5.33 (s, 0.5H), 5.17 (s, 0.5H), 5.06 (t, 1H), 4.40 (s, 0.5H), 4.11-4.30 (m, 3H), 3.84-4.05 (m, 4H), 3.72 (s, 0.5H), 3.12-3.24 (m, 3H), 2.99 (s, 1H), 2.40-2.68 (m, 2H), 1.81-2.27 (m, 6H), 1.49 (m, 3H). |
| 952a | 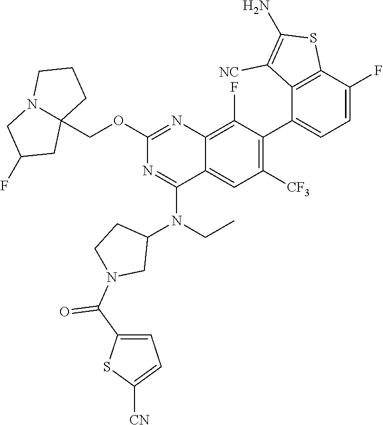 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2ccc(C#N)s2)C1] | |
| 953 | 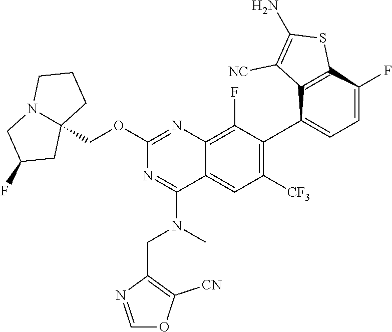 | LCMS m/z [M − 1]: 699.0 ¹H NMR (400 MHz, CD₃OD) δ 8.49 (s, 1H), 8.44 (s, 1H), 7.24-7.19 (m, 1H), 7.00 (t, 1H), 5.36 (s, 0.5H), 5.26-5.19 (m, 1.5H), 5.05-5.01 (m, 1H), 4.25-4.18 (m, 2H), 3.73 (s, 3H), 3.26-3.16 (m, 3H), 3.05-2.98 (m, 1H), 2.41-2.33 (m, 1H), 2.30-2.23 (m, 1H), 2.18-2.12 (m, 1H), 2.02-1.95 (m, 2H), 1.90-1.83 (m, 1H). |
| 953a | 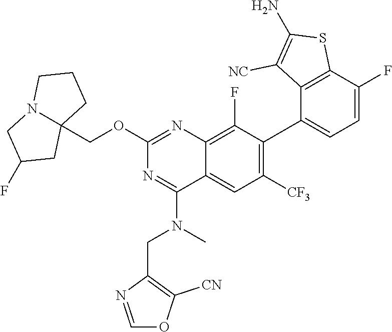 [SMILES: CN(Cc1ncoc1C#N)c1nc(OCC23CCCN2CC(F)C3)nc2c(F)c(-c3ccc(F)c4sc(N)c(C#N)c34)c(C(F)(F)F)cc12] | |
| 954 | 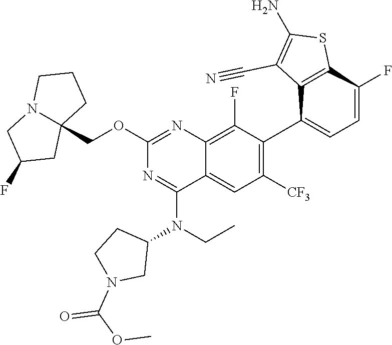 | LCMS m/z [M − 1]: 734.3 ¹H NMR (400 MHz, CD₃OD) δ 8.28 (s, 1H), 7.21-7.25 (m, 1H), 7.02 (t, J = 8.88 Hz, 1H), 5.51 (d, J = 50.84 Hz, 1H), 5.00-5.04 (m, 1H), 4.67-4.75 (m, 2H), 3.43-4.02 (m, 13H), 2.10-2.66 (m, 8H), 1.46-1.50 (m, 3H). |
| 955 | 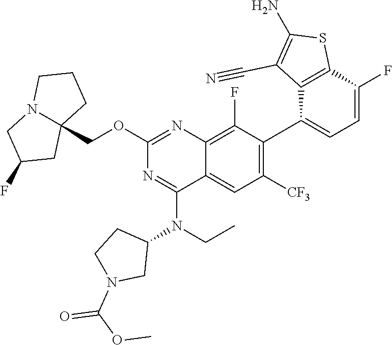 | LCMS m/z [M − 1]: 734.3 ¹H NMR (400 MHz, CD₃OD) δ 8.24 (s, 1H), 7.21-7.24 (m, 1H), 7.01 (t, J = 9.14 Hz, 1H), 5.35 (d, J = 50.84 Hz, 1H), 5.05-5.09 (m, 1H), 4.38-4.57 (m, 2H). 3.44-3.93 (m, 10H), 2.84-3.17 (m, 3H), 1.85-2.85 (m, 8H), 1.43- 1.46 (m, 3H). |
| 956 | 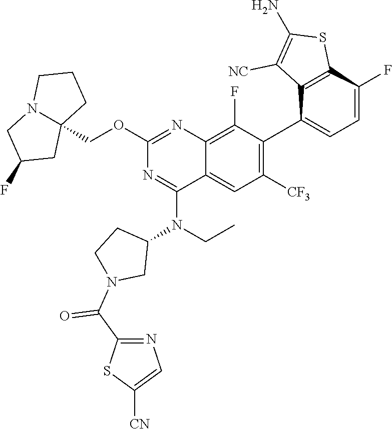 | LCMS m/z [M − 1]: 812.1 ¹H NMR (400 MHz, CD₃OD): δ 8.52-8.59 (m, 1 H), 8.26 (s, 1 H), 7.16-7.24 (m, 1 H), 6.95-7.04 (m, 1 H), 5.32 (s, 0.5 H), 5.19 (s, 0.5 H), 5.00-5.13 (m, 1 H), 4.66-4.81 (m, 1 H), 4.06-4.32 (m, 4 H), 3.85-3.94 (m, 2 H), 3.65- 3.79 (m, 1 H), 3.10-3.24 (m, 3 H), 2.95-3.01 (m, 1 H), 2.40-2.62 (m, 2 H), 2.21-2.36 (m, 1 H), 2.14-2.21 (m, 1H), 2.02-2.10 (m, 1 H), 1.79-1.98 (m, 3 H), 1.41-1.53 (m, 3 H). |
| 956a | 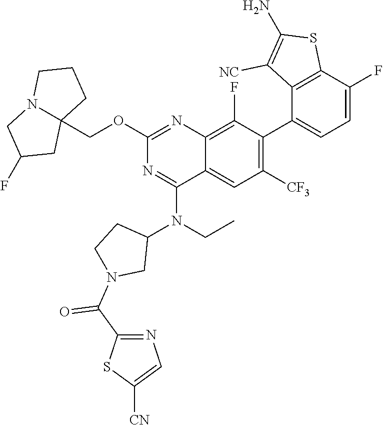 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2ncc(C#N)s2)C1] | |
| 957 | 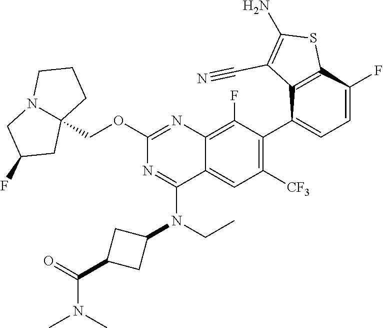 | LCMS m/z [M − 1]: 732.1 ¹H NMR (400 MHz, CD₃OD): δ 8.19 (s, 1 H), 7.17-7.25 (m, 1 H), 6.96-7.05 (s, 1 H), 5.39 (s, 0.5 H), 5.26 (s, 0.5 H), 4.69-5.81 (m, 1 H), 4.22-4.40 (m, 2 H), 3.86-4.00 (m, 2 H), 3.18-3.28 (m, 2 H), 3.05 (s, 3 H), 2.95 (s, 3 H), 2.67-2.79 (m, 2 H), 2.47-2.65 (m, 2 H), 2.11-2.37 (m, 3 H), 1.88-2.05 (m, 3 H), 1.35-1.39 (m, 3 H). |
| 958 | 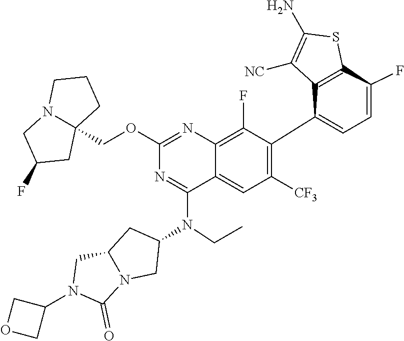 | LCMS m/z [M − 1]: 787.2 ¹H NMR (400 MHz, CD₃OD) δ 8.19 (s, 1H), 7.19-7.22 (m, 1H), 7.01 (t, 1H), 5.36 (s, 0.5H), 5.20-5.27 (m, 1.5H), 5.09-5.16 (m, 1H), 4.88-4.90 (m, 2H), 4.71-4.84 (m, 2H), 4.30 (d, 1H), 4.22 (d, 1H), 3.95-4.03 (m, 1H), 3.91 (t, 1H), 3.72-3.80 (m, 2H), 3.56-3.68 (m, 3H), 3.18-3.24 (m, 3H), 2.97-3.04 (m, 1H), 2.33-2.42 (m, 1H), 2.21-2.32 (m, 2H), 2.12-2.16 (m, 1H), 1.94-2.06 (m, 3H), 1.84-1.92 (m, 1H), 1.36 (t, 3H). |
| 958a | 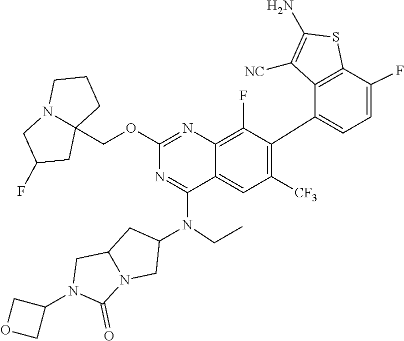 [SMILES: CCN(c1nc(OCC23CCCN2CC(F)C3)nc2c(F)c(-c3ccc(F)c4sc(N)c(C#N)c34)c(C(F)(F)F)cc12)C1CC2CN(C3COC3)C(=O)N2C1] | |
| 959 | 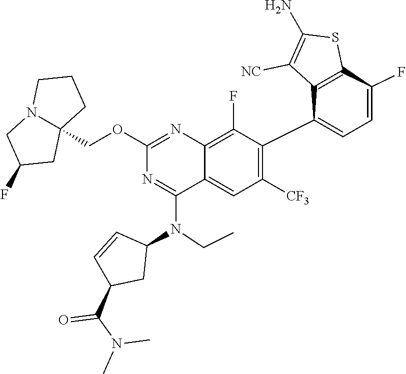 | LCMS m/z [M − 1]: 747.3 ¹H NMR (400 MHz, CD₃OD) δ = 8.22 (s, 1H), 7.24- 7.18 (m, 1H), 7.04-6.98 (m, 1H), 5.42 (s, 0.5H), 5.29 (s, 0.5H), 4.91 (s, 1H), 4.36 (dd, J = 29.9 Hz, 10.9, 2H), 3.87 (d, J = 7.0 Hz, 2H), 3.54-3.44 (m, 1H), 3.44-3.32 (m, 3H), 3.13 (d, J = 9.9 Hz, 4H), 2.96 (d, J = 8.0 Hz, 3H), 2.47-1.98 (m, 12H), 1.44 (t, J = 6.9 Hz, 3H). |
| 960 | 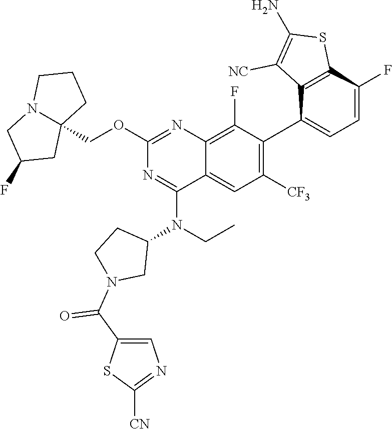 | LCMS m/z [M − 1]: 812.1 ¹H NMR (400 MHz, CD₃OD) δ = 8.53 (d, J = 14.1 Hz, 1H), 8.27 (s, 1H), 7.22 (s, 1H), 7.06-6.97 (m, 1H), 5.34 (d, J = 4.5 Hz, 0.5H), 5.21 (s, 0.5H), 5.08 (dd, J = 15.6 Hz, 7.8 Hz, 1H), 4.41-4.17 (m, 3H), 3.89 (dddd, J = 56.4 Hz, 52.6 Hz, 34.2 Hz, 10.1 Hz, 5H), 3.28- 2.94 (m, 4H), 2.68-2.43 (m, 2H), 2.34-1.88 (m, 6H), 1.54-1.42 (m, 3H). |
| 960a | 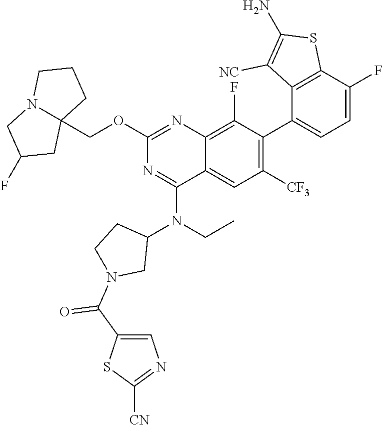 [SMILES: CCN(c1nc(OCC23CCCN2CC(F)C3)nc2c(F)c(-c3ccc(F)c4sc(N)c(C#N)c34)c(C(F)(F)F)cc12)C1CCN(C(=O)c2cnc(C#N)s2)C1] | |
| 961 | 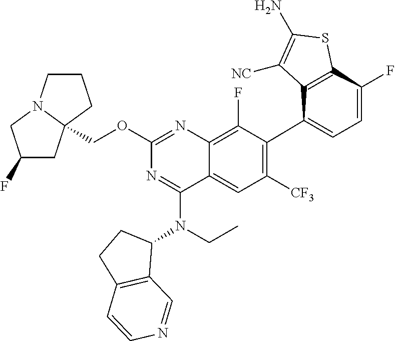 | LCMS m/z [M − 1]: 710.1 ¹H NMR (400 MHz, CD₃OD) δ 8.53 (s, 1H), 8.43 (d, J = 5.1 Hz, 1H), 8.39 (s, 1H), 7.45 (d, J = 5.1 Hz, 1H), 7.22 (dd, J = 8.4, 5.0 Hz, 1H), 7.05-6.96 (m, J = 9.4, 8.4 Hz, 1H), 6.56 (t, J = 8.1 Hz, 1H), 5.34 (s, 0.5H), 5.21 (d, J = 2.0 Hz, 0.5H), 4.30-4.18 (m, J = 10.4 Hz, 2H), 3.26 (s, 3H), 3.22 (d, J = 2.8 Hz, 1H), 3.19 (s, 1H), 3.13 (d, J = 1.6 Hz, 1H), 3.11-3.05 (m, 1H), 3.02- 2.94 (m, J = 9.4, 5.5 Hz, 1H), 2.79-2.71 (m, J = 12.3, 8.8, 4.4 Hz, 1H), 2.48-2.04 (m, 5H), 2.01-1.92 (m, J = 12.7, 6.5 Hz, 2H), 1.90-1.81 (m, J = 13.9, 7.0 Hz, 1H). |
| 961a | 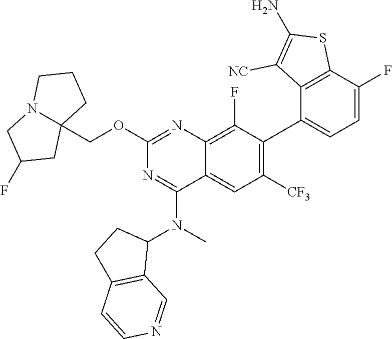 [SMILES: CN(c1nc(OCC23CCCN2CC(F)C3)nc2c(F)c(-c3ccc(F)c4sc(N)c(C#N)c34)c(C(F)(F)F)cc12)C1CCc2ccncc21] | |
| 962 | 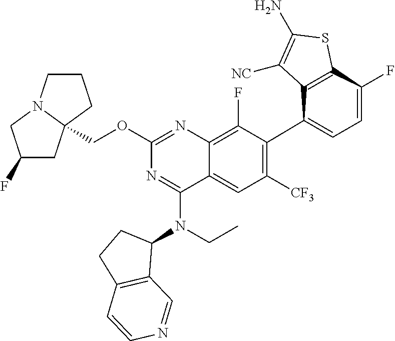 | LCMS m/z [M − 1]: 710.1 ¹H NMR (400 MHz, CD₃OD) δ 8.53 (s, 1H), 8.43 (d, J = 5.1 Hz, 1H), 8.40 (s, 1H), 7.45 (d, J = 5.1 Hz, 1H), 7.27-7.16 (m, J = 8.3, 5.0 Hz, 1H), 7.01 (t, J = 9.4, 8.4 Hz, 1H), 6.55 (t, J = 8.0 Hz, 1H), 5.35 (s, 0.5H), 5.22 (s, 0.5H), 4.31 (d, J = 10.4 Hz, 1H), 4.20 (d, J = 10.4 Hz, 1H), 3.31 (d, J = 1.6 Hz, 1H), 3.25 (s, 3H), 3.21 (d, J = 4.1 Hz, 1H), 3.16 (s, 1H), 3.14-3.07 (m, 1H), 3.03- 2.95 (m, J = 9.5, 5.6 Hz, 1H), 2.84-2.73 (m, 1H), 2.45- 2.06 (m, 5H), 2.00-1.90 (m, J = 16.2, 7.3 Hz, 2H), 1.89-1.81 (m, 1H). |
| 963 | 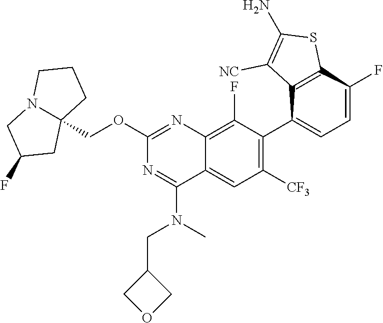 | LCMS m/z [M − 1]: 663.0 ¹H NMR (400 MHz, CD₃OD) δ 8.49 (s, 1H), 7.26-7.19 (m, 1H), 7.08-7.02 (m, 1H), 5.65 (s, 0.5H), 5.51 (s, 0.5H), 4.99-4.89 (m, 1H), 4.85-4.84 (m, 1H), 4.55- 4.43 (m, 1H), 4.09-3.91 (m, 4H), 3.90-3.85 (m, 3H), 3.85-3.72 (m, 4H), 3.53-3.44 (m, 1H), 2.80-2.69 (m, 1H), 2.68-2.55 (m, 2H), 2.50-2.31 (m, 3H), 2.22- 2.13 (m, 1H). |
| 963a | 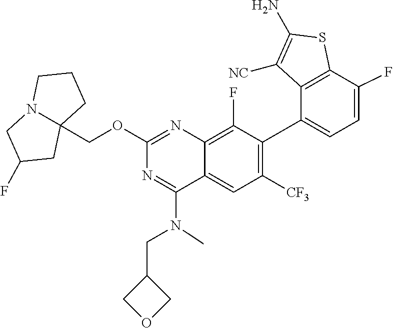 [SMILES: CN(CC1COC1)c1nc(OCC23CCCN2CC(F)C3)nc2c(F)c(-c3ccc(F)c4sc(N)c(C#N)c34)c(C(F)(F)F)cc12] | |
| 964 | 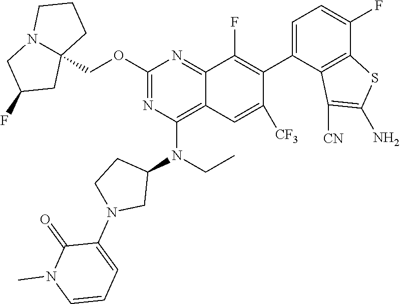 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@@H]1CCN(c2cccn(C)c2=O)C1] | LCMS m/z [M − 1]: 783.1 ¹H NMR (400 MHz, CD₃OD) δ 8.32 (s, 1H), 7.20-7.28 (m, 2H), 7.05 (t, J = 8.8 Hz, 1H), 6.70-6.77 (m, 1H), 6.34 (t, J = 7.2 Hz, 1H), 5.57 (d, J = 52.0 Hz, 1H), 5.08- 5.13 (m, 1H), 4.66-4.81 (m, 2H), 3.77-4.26 (m, 6H), 3.44-3.61 (m, 5H), 3.15-3.27 (m, 2H), 2.15-2.76 (m, 4H), 2.21-2.31 (m, 3H), 2.01-2.05 (m, 1H), 1.53 (t, J = 6.8 Hz, 3H). |
| 964a | 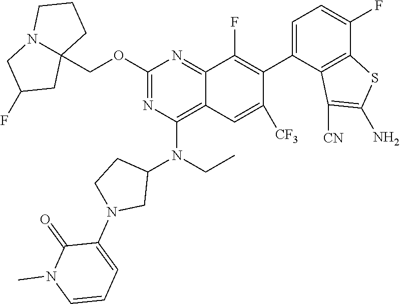 [SMILES: CCN(c1nc(OCC23CCCN2CC(F)C3)nc2c(F)c(-c3ccc(F)c4sc(N)c(C#N)c34)c(C(F)(F)F)cc12)C1CCN(c2cccn(C)c2=O)C1] | |
| 965 | 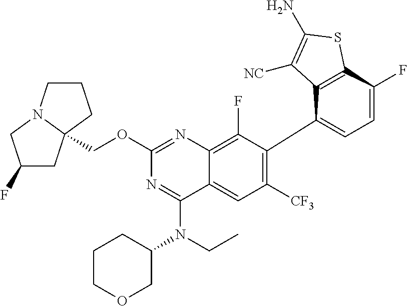 | LCMS m/z [M − 1]: 691.1 ¹H NMR (400 MHz, CD₃OD) δ 8.20 (s, 1H), 7.24-7.20 (m, 1H), 7.01 (t, J = 8.8 Hz, 1H), 5.40 (s, 1H), 5.26 (s, 1H), 4.87 (s, 1H), 4.61-4.56 (m, 1H), 4.32 (dd, J = 23.4, 10.7 Hz), 4.08-4.06 (m, 1H), 3.92-3.76 (m, 4H), 3.48- 3.38 (m, 2H), 3.31-3.25 (M, 1H), 3.09-3.02 (m, 1H), 2.42-2.15 (m, 5H), 2.06-1.80 (m, 5H), 1.42 (t, J = 6.9 Hz, 3H). |
| 965a | 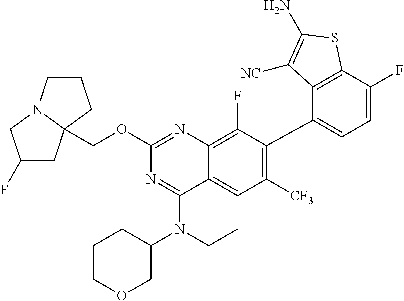 | |
| 966 | 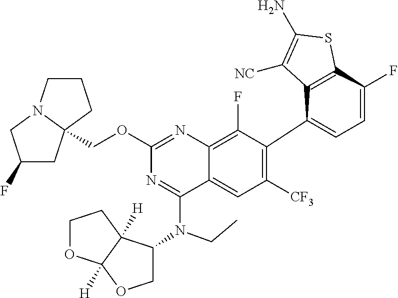 | LCMS m/z [M − 1]: 719.1 ¹H NMR (400 MHz, CD₃OD) δ 8.52 (brs, 0.18H), 8.23 (s, 1H), 7.21 (t, J = 3.2 Hz, 1H), 7.01 (t, J = 9.2 Hz, 1H), 5.98 (d, J = 5.2 Hz, 1H), 5.43-5.30 (m, 1H), 4.92-4.91 (m, 1H), 4.44 (d, J = 10.8 Hz, 1H), 4.33 (d, J = 10.8 Hz, 1H), 4.28-4.14 (m, 2H), 4.00-3.80 (m, 4H), 3.54-3.33 (m, 3H), 3.26-3.21 (m, 1H), 3.16-3.06 (m, 1H), 2.48- 1.89 (m, 8H), 1.44 (t, J = 7.2 Hz, 1H). |
| 966a | 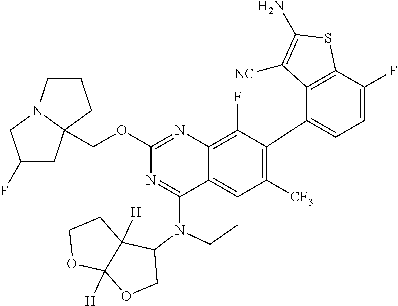 [SMILES: CCN(c1nc(OCC23CCCN2CC(F)C3)nc2c(F)c(-c3ccc(F)c4sc(N)c(C#N)c34)c(C(F)(F)F)cc12)C1COC2OCCC21] | |
| 967 | 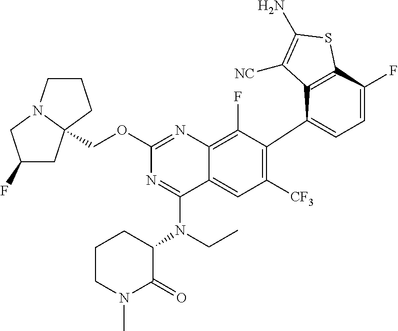 | LCMS m/z [M − 1]: 718.2 ¹H NMR (400 MHz, CD₃OD) δ = 8.24 (s, 1H), 7.23 (dd, J = 8.3, 5.1, 1H), 7.02 (dd, J = 9.4, 8.5, 1H), 5.44 (s, 0.5H), 5.31 (s, 0.5H), 4.84-4.78 (m, 1H), 4.33-4.24 (m, 2H), 3.97 (d, J = 6.1, 2H), 3.54 (ddd, J = 57.0, 22.4, 8.4, 5H), 3.15 (d, J = 10.9, 1H), 3.02 (s, 3H), 2.67-2.55 (m, 1H), 2.19 (ddt, J = 46.1, 39.9, 20.2, 9H), 1.61 (t, J = 6.9, 3H). |
| 967a | 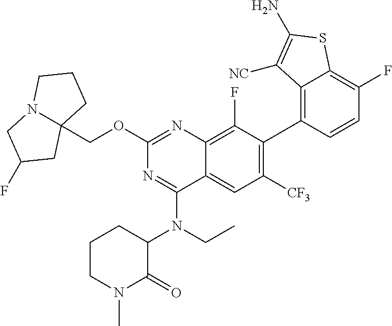 [SMILES: CCN(c1nc(OCC23CCCN2CC(F)C3)nc2c(F)c(-c3ccc(F)c4sc(N)c(C#N)c34)c(C(F)(F)F)cc12)C1CCCN(C)C1=O] | |
| 968 | 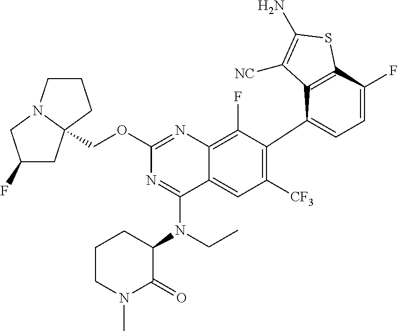 | LCMS m/z [M − 1]: 718.2 ¹H NMR (400 MHz, CD₃OD) δ = 8.26 (s, 1H), 7.19 (dd, J = 8.3, 5.1, 1H), 7.04-6.97 (m, 1H), 5.47 (s, 0.5H), 5.34 (s, 0.5H), 4.84-4.78 (m, 1H), 4.46 (d, J = 11.2, 1H), 4.21 (d, J = 10.9, 1H), 3.99 (d, J = 7.1, 2H), 3.68- 3.36 (m, 5H), 3.17 (d, J = 31.6, 1H), 3.01 (d, J = 5.8, 3H), 2.58-1.96 (m, 10H), 1.60 (t, J = 6.8 Hz, 3H). |
| 969 | 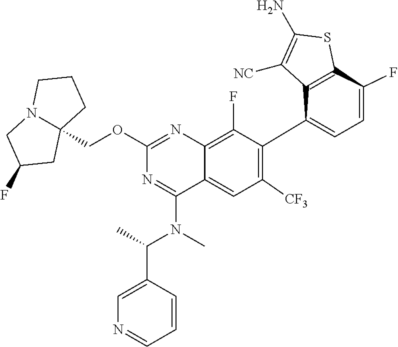 | LCMS m/z [M − 1]: 699.2 ¹H NMR (400 MHz, CD₃OD) δ 8.63 (s, 1H), 8.50 (d, 1H), 8.31 (s, 1H), 7.94 (t, 1H), 7.47-7.50 (m, 1H), 7.19- 7.23 (m, 1H), 7.01 (t, 1H), 6.26-6.33 (m, 1H), 5.35 (s, 0.5H), 5.21 (s, 0.5H), 4.16-4.25 (m, 2H), 3.35-3.48 (m, 2H), 3.16-3.26 (m, 3H), 3.06-3.35 (m, 1H), 2.95-3.02 (m, 1H), 2.13-2.36 (m, 3H), 2.06-2.02 (m, 1H), 1.91- 1.99 (m, 2H), 1.84 (d, 3H). |
| 969a | 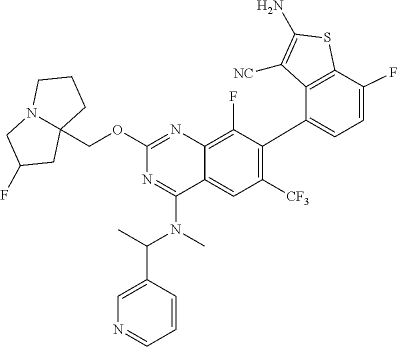 [SMILES: CC(c1cccnc1)N(C)c1nc(OCC23CCCN2CC(F)C3)nc2c(F)c(-c3ccc(F)c4sc(N)c(C#N)c34)c(C(F)(F)F)cc12] | |
| 970 | 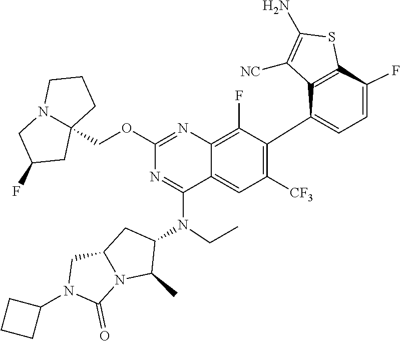 | LCMS m/z [M − 1]: 799.2 ¹H NMR (400 MHz, CD₃OD) δ 8.24 (s, 1H), 7.28- 7.20 (m, J = 8.3, 5.0 Hz, 1H), 7.07-6.97 (m, 1H), 5.38 (s, 0.5H), 5.24 (s, 0.5H), 4.86-4.80 (m, J = 6.6 Hz, 1H), 4.49-4.39 (m, J = 8.5 Hz, 1H), 4.31 (d, J = 10.6 Hz, 1H), 4.22 (d, J = 10.6 Hz, 1H), 4.13-4.08 (m, 1H), 4.04-3.97 (m, 1H), 3.86-3.78 (m, 1H), 3.74-3.61 (m, J = 14.7, 8.4 Hz, 2H), 3.56-3.52 (m, J = 9.5, 2.2 Hz, 1H), 3.24 (d, J = 13.0 Hz, 2H), 3.06-2.98 (m, 1H), 2.38-2.21 (m, 5H), 2.17-2.06 (m, J = 21.2, 9.7 Hz, 4H), 2.05-1.95 (m, 3H), 1.95-1.87 (m, J = 13.6, 8.8 Hz, 1H), 1.75-1.68 (m, 2H), 1.36 (t, J = 6.9 Hz, 3H), 1.30 (t, J = 6.2 Hz, 3H). |
| 970a | 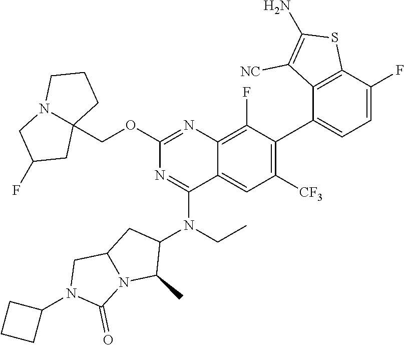 [SMILES: CCN(c1nc(OCC23CCCN2CC(F)C3)nc2c(F)c(-c3ccc(F)c4sc(N)c(C#N)c34)c(C(F)(F)F)cc12)C1CC2CN(C3CCC3)C(=O)N2[C@@H]1C] | |
| 971 | 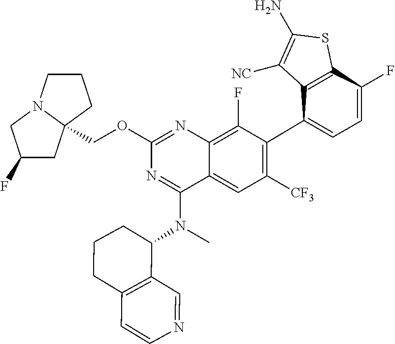 | LCMS m/z [M − 1]: 724.2 ¹H NMR (400 MHz, CD₃OD) δ 8.39 (d, J = 6.1 Hz, 2H), 8.31 (d, J = 5.2 Hz, 1H), 7.28 (d, J = 5.2 Hz, 1H), 7.25-7.20 (m, 1H), 7.05-6.98 (m, 1H), 6.29-6.18 (m, 1H), 5.32 (s, 0.5H), 5.19 (s, 0.5H), 4.21 (s, 2H), 3.35 (s, 3H), 3.21 (s, 2H), 3.14 (d, J = 6.7 Hz, 1H), 3.05- 2.87 (m, 3H), 2.39-2.23 (m, 2H), 2.23-2.09 (m, 4H), 2.03-1.91 (m, 3H), 1.90-1.78 (m, 1H). |
| 971a | 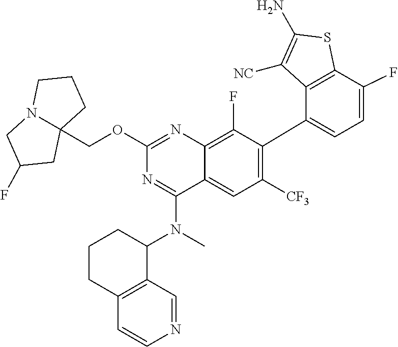 [SMILES: CN(c1nc(OCC23CCCN2CC(F)C3)nc2c(F)c(-c3ccc(F)c4sc(N)c(C#N)c34)c(C(F)(F)F)cc12)C1CCCc2ccncc21] | |
| 972 | 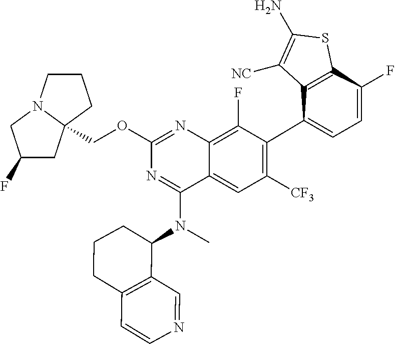 | LCMS m/z [M − 1]: 724.2 ¹H NMR (400 MHz, CD₃OD) δ 8.42 (s, 1H), 8.35- 8.30 (m, 2H), 7.28 (d, J = 5.2 Hz, 1H), 7.26-7.20 (m, 1H), 7.07-6.98 (m, 1H), 6.30 (d, J = 10.2 Hz, 1H), 5.39 (s, 0.5H), 5.26 (s, 0.5H), 4.41-4.13 (m, 2H), 3.44- 3.32 (m, 6H), 3.10-2.87 (m, 3H), 2.43-2.30 (m, 2H), 2.27 (d, J = 8.3 Hz, 1H), 2.23-2.11 (m, 3H), 2.07- 1.92 (m, 3H), 1.90-1.78 (m, 1H). |
| 973 | 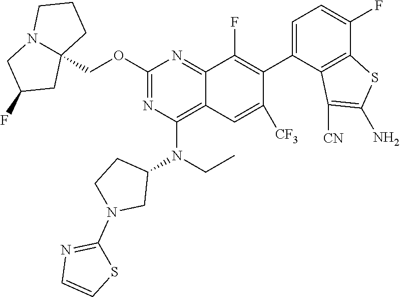 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@H]1CCN(c2nccs2)C1] | LCMS m/z [M − 1]: 759.0 ¹H NMR (400 MHz, CD₃OD) δ 8.31 (s, 1H), 7.30-7.37 (m, 1H), 7.21-7.25 (m, 1H), 7.03 (t, J = 9.1 Hz, 1H), 6.90-6.95 (m, 1H), 5.56 (d, J = 53.2 Hz, 1H), 5.17-5.24 (m, 1H), 4.57-4.72 (m, 2H), 3.69-4.11 (m, 9H), 3.41- 3.48 (m, 1H), 2.48-2.86 (m, 4H), 1.99-2.35 (m, 4H), 1.51-1.60 (m, 3H). |
| 973a | 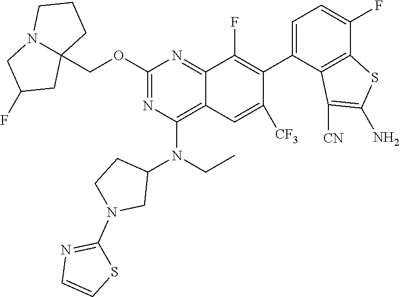 [SMILES: CCN(c1nc(OCC23CCCN2CC(F)C3)nc2c(F)c(-c3ccc(F)c4sc(N)c(C#N)c34)c(C(F)(F)F)cc12)C1CCN(c2nccs2)C1] | |

### Biological Example 1: Disrupting RAS-Effector Binding HTRF Assay

A protein:protein interaction (PPI) Homogeneous Time Resolved Fluorescence (HTRF) assay was used to determine the effectiveness of compounds of the present disclosure in disrupting KRAS protein and effector RAF1 binding.

The HTRF assay used the following reagents and proteins: (i) 50 nM Avi-KRAS G12D (1-169) GppNHp/RAF1 RBD-3×FLAG (52-151); (ii) 50 nM Avi-KRAS G12R (1-169) GppNHp/RAF1 RBD-3×FLAG (52-151); (iii) 50 nM Avi-KRAS G12V (1-169) GppNHp/RAF1 RBD-3×FLAG (52-151); (iv) 50 nM Avi-KRAS WT (1-169) GppNHp/RAF1 RBD-3×FLAG (52-151); and (v) 75 nM Avi-RAF1 RBD-3×FLAG. The assay buffer included: 50 mM Tris pH 7.5, 100 mM NaCl, 5 mM MgCl₂, 0.1% BSA, 0.01% Tween, and 10% DMSO. The bead buffer included: 50 mM Tris pH 7.5, and 0.01% Tween 20. Assay volume of 20 μL was used in a 384 well plate-low volume format. Compound titration: 30-0.0045 μM, 3× dilution series.

The HTRF assay employed the following protocol:

Compounds were dispensed in assay plate (384-well, Grenier Bione #784075) using Echo (model 555) with dose response settings: 200 nL final volume, titration from 30 μM as a 10-point dilution series. Protein mix was prepared in assay buffer, and dispensed on plates, 10 μL per well, then incubated for 1 h at room temperature, with 700 rpm shaking. Reagents mix was prepared and dispensed on plates, 10 μL per well, then incubated for 1 h at room temperature, with 700 rpm shaking.

Plates were analyzed on an Envision plate reader using the following setting: Excitation 320 nM, Bandwidth 75 nM; Emission 615 nM, Bandwidth 85 nM, Gain 100%, Flashes 100, Lag 60 μs. Data was reported as percentage of activity, with DMSO as 100%. Data was plotted and analyzed using Prism 8.

The biochemical Raf1-KRAS G12D/G12R/G12V/WT-GppNHP disruption assay IC₅₀ of selected compounds described herein are shown in Table 11.

### Biological Example 2: Cell-Based pERK HTRF Assay in GP2d Cell

A pERK assay (Perkin Elmer) was used to determine the effectiveness of compounds in disrupting KRAS G12D protein/effector signaling in cells.

On Day 1, cells (GP2d) were seeded into 96-well plates at 3×10⁴ cells/well in 80 μL complete growth media (DMEM, 10% FBS).

On Day 2, cells were treated with compounds at 0.25% DMSO. The source plate was created with compounds diluted in media at 5-fold the final assay concentration. The compounds were run in a 9-point concentration curve starting at 1 μM or in a 9-point concentration curve starting at 100 nM, with a half-log dilution between concentrations. Twenty μL was transferred onto the cell plates (final volume in wells was 100 μL). Plates were harvested after 60 min incubation by aspirating media and adding kit-supplied 1× supplemented lysis buffer to all wells (50 μL per well). Plates were then placed on a plate shaker and incubated at 850 rpm for an additional 30 min.

Antibody mixture solution was prepared by diluting aliquoted d2 and Eu Cryptate antibodies 1:20 in kit supplied detection buffer, and mixing the diluted antibodies solutions (1:1 v:v). Four μL of this solution was then added to a 384-well detection plate (Perkin Elmer, 6008230).

Samples were homogenized by pipetting up and down and then transferred (16 μL of cell lysates) from the 96-well cell culture plate to two wells of the HTRF 384-well detection plate containing the antibody solution. Plates were centrifuged (524 g for 1 min) and allowed to incubate between 4 and 24 h at room temperature. Maximum signal is reached after 4 h incubation time and remains stable over a period of 24 hours. Therefore, readings can be made between 4 and 24 h of incubation. Plates were centrifuged again (524 g for Imin), then analyzed on the EnVision plate reader using the following settings: Excitation 320 nm, Bandwidth 75 nm; Emission 615 nm, Bandwidth 85 nm; Gain 100%; Flashes 100; Lag 60 μs.

The Cell-based pERK HTRF assay in GP2d cell IC₅₀ of selected compounds described herein are shown in Table 11 and Table 12.

### Biological Example 3: Disrupting RAS-Effector Binding HTRF Assay

A protein:protein interaction (PPI) Homogeneous Time Resolved Fluorescence (HTRF) assay was used to determine the effectiveness of compounds in disrupting KRAS protein and effector (RAF1) binding.

The HTRF assay used the following reagents and proteins: (i) 50 nM Avi-KRAS G12D (2-169) GTP/RAF1 RBD-3×FLAG (51-131); (ii) 50 nM Avi-KRAS G12C (2-169) GTP/RAF1 RBD-3×FLAG (51-131); (iii) 50 nM Avi-KRAS G12V (2-169) GTP/RAF1 RBD-3×FLAG (51-131); (iv) 50 nM Avi-KRAS WT (2-169) GTP/RAF1 RBD-3×FLAG (51-131); and (v) 75 nM 3×FLAG-RAF1 RBD (51-131)-Avi. The assay buffer included: 50 mM Tris pH 7.5 (Invitrogen, 15567-027), 100 mM NaCl (Sigma, S5150-1L), 5 mM MgCl₂ (Sigma, 63069), 0.1% BSA (Sigma, A7030), 0.01% Tween 20 (Sigma, P7949), 10% DMSO (Cell Signaling, 12611). The bead buffer included: 50 mM Tris pH 7.5 (Invitrogen, 15567-027), 0.01% Tween 20 (Sigma, P7949). An assay volume of 20 μL was used in a 384 well plate-low volume format. Compound titration: 30-0.005 μM, 3× dilution series.

The HTRF assay employed the following protocol:

Compounds were dispensed in assay plate (384-well, Grenier Bio-One #784075) using Echo (model 555) with dose response settings: 200 nL final volume, titration from 30 μM as a 10-point dilution series. Proteins and HTRF reagents were mixed and dispensed onto plates, 20 μL per well, and then incubated for 1 h at room temperature, with 700 rpm shaking.

Plates were analyzed on an Envision plate reader using the following setting: Excitation 320 nm, Bandwidth 75 nm; Emission 615 nm, Bandwidth 85 nm; Gain 100%; Flashes 100; Lag 60 μs. Data was reported as percentage of activity, with DMSO as 100%. Data was plotted and analyzed using Prism 8.

The biochemical Raf1-KRAS G12D/G12V/G12C/WT-GTP disruption assay IC₅₀ of selected compounds described herein are shown in Table 12.

### Biological Example 4: Cell-Based pERK HTRF Assay in SW620 Cell

A pERK assay (Perkin Elmer) was used to determine the effectiveness of compounds in disrupting KRAS G12V protein/effector signaling in cells.

On Day 1, cells (SW620) were seeded into 96-well plates at 3×10⁴ cells/well in 80 μL complete growth media (RPMI, 10% FBS).

On Day 2, cells were treated with compounds at 0.25% DMSO. The source plate was created with compounds diluted in media at 5-fold the final assay concentration. The compounds were run in a 9-point concentration curve starting at 1 μM, with a half-log dilution between concentrations. Twenty μL was transferred onto the cell plates (final volume in wells was 100 μL). Plates were harvested after 4 h incubation by aspirating media and adding kit-supplied 1× supplemented lysis buffer to all wells (50 μL per well). Plates were then placed on a plate shaker and incubated at 850 rpm for an additional 30 min.

Antibody mixture solution was prepared by diluting aliquoted d2 and Eu Cryptate antibodies 1:20 in kit supplied detection buffer and mixing the diluted antibodies solutions (1:1 v:v). Four μL of this solution was then added to a 384-well detection plate (Perkin Elmer, 6008230).

Samples were homogenized by pipetting up and down and then transferred (16 μL of cell lysates) from the 96-well cell culture plate to two wells of the HTRF 384-well detection plate containing the antibody solution. Plates were centrifuged (524 g for 1 min) and allowed to incubate between 4 and 24 h at room temperature. Maximum signal was reached after 4 h incubation time and remained stable over a period of 24 hours. Therefore, readings could be made between 4 and 24 h of incubation. Plates were centrifuged again (524 g for imin), then analyzed on the EnVision plate reader using the following settings: Excitation 320 nm, Bandwidth 75 nm; Emission 615 nm, Bandwidth 85 nm; Gain 100%; Flashes 100; Lag 60 μs.

The Cell-based pERK HTRF assay in SW620 cell IC₅₀ of selected compounds described herein are shown in Table 11 and Table 12.

### Biological Example 5: Cell-Based pERK HTRF Assay in AsPC-1, SW1900, HPAC, Capan-2, RKN, H358, HCT116, KP-2, H1573, A549, MKN1, and HT1080 Cells

The inhibition of ERK phosphorylation, as measured using the HTRF Advanced Phospho-ERK1/2 (Thr202/Tyr204) Detection Assay kit (Perkin Elmer), was used to determine the effectiveness of test compounds in disrupting KRAS-driven protein/effector signaling m cells.

On day 1, cells were seeded into 96-well plates at 2.5×10⁴ cells/well in 100 μL of complete growth media supplemented with 10% fetal bovine serum (FBS) and incubated at 37° C. in the presence of 5% CO₂.

On day 2, cells were treated with compounds diluted in DMSO at 0.25% final DMSO concentration. Compounds were dosed in a 9-point concentration curve with three-fold dilution between concentrations. Cells were incubated in the presence of compound for two hours at 37° C. Following this treatment period, cells were harvested by aspirating the media, adding 40 μL of kit-supplied 1× lysis buffer per well, and shaking for 30 minutes at 250 rpm at room temperature.

Following cell lysis, samples were homogenized by pipetting up and down twenty times and 16 μL of cell lysate per well was transferred from the 96-well cell culture plate to two wells of a 384-well detection plate. A complete detection buffer was prepared by diluting D2 and Eu Cryptate antibodies 1:20 in kit-provided detection solution. 4 μL of complete detection buffer was then added to each well with lysate. Plates were incubated with gentle rocking for 24 hours at 4° C.

Following this incubation period, plates were centrifuged at 500×g for one minute and analyzed on a CLARIOstar plate reader. Data was imported into Microsoft Excel and the signal from the 665 nm channel was divided by the signal from the 620 nm channel. % pERK was calculated as:

% Signal=100*((Sample Value−average of positive control)/(Vehicle Value−average of positive control))

Data was imported to the Prism software package (Graphpad) and IC₅₀ values were calculated using the log (inhibitor) vs. response—variable slop (Four parameters) model.

The cell-based pERK HTRF assay in the titled cells IC₅₀ of selected compounds described herein are shown in Table 13.

### Biological Example 6: 3D Cell Viability Assay in AsPC-1, SW1900, HPAC, Capan-2, RKN, H358, HCT116, KP-2, H1573, A549, MKN1, and HT1060 Cells

The inhibition of cellular viability as a result of test compound treatment in a spheroid assay was measured using the CellTiter-Glo® 3D Cell Viability Assay (Promega).

On Day 1, 1000 cells per well were seeded in 150 μL of complete growth media supplemented with 10% fetal bovine serum (FBS) and incubated at 37° C. in the presence of 5% CO₂ in 96-well ultra-low attachment microplates (Corning) for 48 hours undisturbed to allow spheroids to form.

Following spheroid formation, cells were treated with compounds diluted in DMSO at 0.25% final DMSO concentration. Compounds were dosed in a 9-point concentration curve with three-fold dilution between concentrations. Cells were incubated in the presence of compound for 72 hours at 37° C. in the presence of 5% CO₂.

Following this incubation period, the kit-provided CTG reagent was allowed to equilibrate for 30 minutes at room temperature. 150 μL of equilibrated CTG reagent was added to each well, and the plate was sealed before shaking at 250 rpm for 10 minutes at room temperature to lyse the cells. 150 μL of lysate per well was transferred to a 96-well plate and luminescence was read on a CLARIOstar plate reader.

Data was imported into the Prism software package (Graphpad) and ECs values were calculated using the log (inhibitor) vs. response-variable slope (Four parameters) model.

The 3D viability assay in the titled cells IC₅₀ of selected compounds described herein are shown in Table 14.

For Raf1-KRAS G12D, G12V, G12R, and wild-type KRAS-GppNHP disruption assay, and for Raf1-GTP-KRAS G12D, G12V, G12C, and wild-type disruption assay: A: IC₅₀≤0.1 μM; B: 0.1 μM<IC₅₀≤1 μM; C: 1 μM<IC₅₀≤10 μM; D: IC₅₀>10 μM. For GP2d (KRAS G12D), SW620 (KRAS G12V), AsPC-1 (KRAS G12D), SW1900 (KRAS G12D), HPAC (KRAS G12D), Capan-2 (KRAS G12V), RKN (KRAS G12V). H358 (KRAS G12C), HCT116 (KRAS G13D), KP-2 (KRAS G12R), H1573 (KRAS G12A), A549 (KRAS G12S), MKN1 (KRAS amplification), and HT1080 (NRAS) pERK HTRF assay, and for 3-D viability assay: A: IC₅₀≤0.1 μM; B: 0.1 μM<IC₅₀≤1 μM; C: IC₅₀>1 μM. Blanks in the table represent that a compound was not tested in the indicated assay.

TABLE 11
Biological characterization of selected compounds of the present disclosure.

| | Raf1- | Raf1- | Raf1- | Raf1- | | |
|---|---|---|---|---|---|---|
| | GppNHp- | GppNHp- | GppNHp- | GppNHp- | | |
| Compound | KRAS G12D | KRAS G12V | KRAS G12R | KRAS WT | pERK- | pERK- |
| No. | disruption | disruption | disruption | disruption | Gp2D- | SW620 |
| 1 | C | D | | C | | |
| 2 | B | C | | B | C | |
| 3 | C | C | | C | | |
| 4 | C | D | D | C | | |
| 5 | D | D | | D | | |
| 6 | C | D | | C | | |
| 7 | C | C | | C | | |
| 8 | B | B | C | B | B | |
| 9 | C | D | | C | | |
| 10 | D | D | D | D | | |
| 11 | D | D | | D | | |
| 12 | D | D | | C | C | |
| 13 | B | C | | B | C | |
| 14 | C | C | | B | C | |
| 15 | C | C | | C | C | |
| 16 | C | C | C | C | C | |
| 17 | D | D | | C | C | |
| 18 | A | B | B | A | A | |
| 19 | A | A | A | A | A | A |
| 20 | C | C | C | C | B | |
| 22 | B | B | B | B | A | |
| 23 | B | B | B | B | A | |
| 24 | B | B | B | B | A | |
| 25 | A | A | A | A | A | |
| 26 | A | B | B | B | A | |
| 27 | B | C | B | B | A | |
| 28 | B | C | C | B | B | |
| 29 | C | C | C | C | B | |
| 30 | B | B | B | B | B | |
| 31 | B | C | B | B | A | |
| 32 | B | C | B | B | B | |
| 33 | B | B | B | B | B | |
| 35 | B | B | B | B | A | |
| 37 | A | B | A | A | A | |
| 39 | B | B | B | B | B | |
| 40 | B | B | B | B | A | |
| 41 | B | B | B | B | A | |
| 43 | B | C | C | B | | |
| 44 | B | B | B | B | A | |
| 45 | B | B | B | B | B | |
| 47 | A | B | B | B | A | |
| 48 | B | B | B | B | A | |
| 49 | A | B | B | B | A | |
| 50 | B | C | C | B | B | |
| 51 | C | D | | C | | |
| 52 | C | C | | C | | |
| 53 | C | D | C | D | | |
| 54 | C | D | | D | | |
| 55 | C | D | D | C | | |
| 56 | C | D | D | C | | |
| 57 | C | D | C | C | | |
| 58 | B | B | | B | B | |
| 60 | C | D | | C | | |
| 61 | C | D | | C | | |
| 62 | C | D | C | C | | |
| 63 | C | D | | C | | |
| 64 | C | C | C | C | | |
| 65 | B | B | B | B | A | |
| 66 | B | B | B | B | B | |
| 67 | C | D | | C | | |
| 68 | D | C | D | D | | |
| 69 | C | C | | C | | |
| 70 | A | B | B | B | A | |
| 71 | A | B | B | B | A | |
| 72 | A | B | B | B | A | |
| 73 | B | B | B | B | A | |
| 74 | A | A | A | A | A | |
| 75 | B | B | B | B | A | |
| 76 | A | B | B | B | A | |
| 77 | A | B | B | B | A | |
| 78 | A | B | B | B | A | |
| 79 | A | B | B | B | A | |
| 80 | A | B | B | B | A | |
| 81 | A | B | A | B | A | |
| 82 | B | C | C | C | B | |
| 83 | B | B | B | B | A | |
| 86 | B | C | B | C | A | |
| 87 | D | D | D | D | D | |
| 88 | B | B | B | B | B | |
| 89 | B | B | B | B | B | |
| 90 | B | B | B | B | A | |
| 91 | B | B | B | B | A | A |
| 93 | B | B | B | B | A | |
| 94 | B | B | B | B | A | |
| 95 | B | B | B | B | A | |
| 96 | C | C | C | C | A | |
| 97 | D | D | | D | | |
| 98 | C | D | | C | B | |
| 99 | C | D | C | C | A | |
| 100 | B | C | B | B | A | |
| 101 | B | C | B | B | A | |
| 102 | B | B | | B | A | |
| 103 | B | B | B | B | A | |
| 104 | B | B | B | B | A | |
| 105 | D | D | D | D | C | |
| 106 | D | D | D | D | C | |
| 107 | D | D | D | D | C | |
| 112 | B | B | B | B | A | A |
| 113 | B | B | B | B | A | |
| 114 | B | B | B | B | A | |
| 115 | A | B | B | B | A | A |
| 116 | B | B | B | B | A | |
| 117 | B | B | B | B | A | |
| 118 | B | B | B | B | A | |
| 119 | C | C | C | B | A | |
| 120 | B | B | B | B | A | |
| 121 | B | B | B | B | B | |
| 122 | B | B | B | B | A | |
| 123 | B | B | B | B | A | |
| 124 | B | B | B | B | B | |
| 125 | B | B | B | B | A | |
| 126 | B | B | B | B | A | |
| 127 | B | B | B | B | A | |
| 128 | B | C | B | B | A | |
| 129 | B | B | B | B | A | |
| 130 | B | C | B | B | A | |
| 131 | B | B | B | B | A | |
| 132 | B | B | B | B | A | |
| 133 | B | B | B | B | A | |
| 134 | B | C | B | B | A | |
| 135 | A | B | B | B | A | |
| 136 | B | C | B | B | A | |
| 137 | B | C | B | B | A | |
| 140 | B | C | C | C | A | |
| 141 | B | C | B | B | A | |
| 142 | B | C | B | B | A | |
| 143 | B | C | C | B | B | |
| 144 | B | C | B | B | A | |
| 145 | B | B | B | B | A | A |
| 146 | B | B | B | B | A | |
| 147 | B | B | B | B | A | A |
| 148 | B | B | B | B | A | A |
| 149 | B | C | C | B | A | |
| 150 | B | C | B | B | A | |
| 151 | B | B | B | B | A | |
| 152 | A | B | B | B | A | |
| 153 | A | B | B | B | A | |
| 154 | B | B | B | B | A | |
| 155 | B | C | B | C | A | |
| 156 | B | B | B | B | A | |
| 157 | B | C | B | B | A | |
| 158 | B | B | B | B | A | |
| 159 | B | C | B | B | A | |
| 160 | B | C | B | B | A | |
| 161 | B | B | B | B | A | A |
| 162 | B | C | B | B | A | A |
| 164 | B | B | B | B | A | |
| 165 | B | B | B | B | A | A |
| 166 | B | B | B | B | A | |
| 167 | B | B | B | B | A | A |
| 168 | A | B | B | A | A | |
| 169 | B | B | B | B | A | |
| 170 | B | B | B | B | A | |
| 171 | B | B | B | B | A | |
| 174 | A | B | A | A | A | |
| 175 | B | B | B | B | A | |
| 176 | A | B | B | B | A | |
| 177 | B | B | B | B | A | |
| 178 | B | C | B | C | A | |
| 179 | B | B | B | B | A | |
| 180 | B | B | B | B | A | |
| 181 | A | B | B | A | A | |
| 182 | B | B | B | B | A | |
| 183 | C | C | C | C | B | |
| 184 | C | D | D | D | B | |
| 186 | B | B | B | B | A | B |
| 187 | A | B | B | B | A | A |
| 189 | B | B | B | B | A | |
| 190 | B | B | B | B | A | B |
| 191 | B | B | B | B | A | |
| 192 | D | D | D | D | C | C |
| 193 | A | A | A | A | A | A |
| 194 | D | D | D | D | C | C |
| 195 | C | C | C | C | B | B |
| 196 | A | B | A | A | A | A |
| 197 | D | D | D | D | C | |
| 198 | B | B | B | B | A | A |
| 199 | D | D | D | D | C | C |
| 200 | C | C | C | C | A | B |
| 201 | B | B | B | B | A | A |
| 202 | B | B | B | B | A | A |
| 203 | B | C | B | B | A | A |
| 204 | C | C | C | C | B | B |
| 205 | B | B | B | B | A | A |
| 206 | C | C | C | C | B | B |
| 207 | B | B | B | B | A | B |
| 208 | B | B | C | B | A | B |
| 209 | B | B | B | B | A | B |
| 210 | C | B | C | B | A | B |
| 211 | B | C | C | B | A | B |
| 212 | B | B | B | B | A | A |
| 213 | B | B | B | B | A | A |
| 214 | C | C | C | C | B | B |
| 215 | B | B | B | B | A | B |
| 216 | B | B | B | B | A | A |
| 217 | B | B | B | B | A | A |
| 218 | B | B | B | B | A | A |
| 220 | D | D | D | D | C | C |
| 221 | B | B | B | B | A | A |
| 222 | B | B | B | B | A | A |
| 223 | B | C | C | B | B | B |
| 224 | B | B | B | B | A | A |
| 226 | D | D | D | D | C | C |
| 227 | C | D | D | D | B | B |
| 228 | B | C | C | B | B | B |
| 229 | B | C | C | C | A | A |
| 230 | B | C | C | B | A | A |
| 231 | C | D | C | C | B | B |
| 232 | A | B | B | B | A | A |
| 233 | B | C | B | B | A | A |
| 234 | B | B | B | B | A | A |
| 235 | B | B | B | B | A | A |
| 236 | B | B | B | B | A | A |
| 237 | B | B | B | B | A | A |
| 238 | D | D | D | D | C | C |
| 239 | D | D | D | D | C | C |
| 240 | C | C | C | C | B | C |
| 241 | B | B | B | B | A | A |
| 242 | B | B | B | B | A | B |
| 243 | B | B | B | B | A | A |
| 244 | B | B | B | B | A | A |
| 245 | A | B | A | A | A | A |
| 246 | B | B | B | B | A | A |
| 247 | B | B | B | B | A | B |
| 248 | B | B | B | B | B | B |
| 249 | B | C | B | B | B | B |
| 250 | B | B | B | B | A | B |
| 251 | B | B | B | B | A | B |
| 252 | C | C | C | C | B | B |
| 253 | B | B | B | B | A | B |
| 254 | B | C | C | B | B | C |
| 255 | B | B | B | B | A | A |
| 256 | A | B | B | B | A | A |
| 257 | B | C | C | B | A | A |
| 258 | A | B | A | A | A | A |
| 259 | B | C | B | B | A | A |
| 260 | A | B | A | A | A | A |
| 261 | B | B | B | A | A | A |
| 262 | A | A | A | A | A | A |
| 263 | A | B | A | A | A | A |
| 264 | B | C | B | B | A | B |
| 265 | B | B | B | B | A | A |
| 266 | A | B | A | A | A | A |
| 267 | A | B | A | B | A | A |
| 268 | A | B | A | A | A | A |
| 269 | B | C | B | B | A | B |
| 270 | B | B | B | B | B | B |
| 271 | B | B | B | B | A | A |
| 272 | B | B | B | B | A | A |
| 273 | B | B | B | B | A | B |
| 274 | B | B | B | B | A | A |
| 275 | B | B | B | B | A | A |
| 276 | C | D | C | C | C | B |
| 277 | B | B | B | B | A | A |
| 278 | B | B | B | B | A | A |
| 279 | A | B | B | B | A | A |
| 280 | B | B | B | B | A | A |
| 281 | B | C | B | B | A | A |
| 282 | A | B | B | A | A | A |
| 283 | B | B | B | B | A | A |
| 284 | B | B | B | B | A | A |
| 285 | B | B | B | B | A | A |
| 286 | A | B | A | A | A | A |
| 287 | B | B | B | B | A | A |
| 288 | D | D | D | D | B | B |
| 289 | B | B | B | B | A | A |
| 290 | B | B | B | B | A | A |
| 291 | B | B | B | B | A | A |
| 292 | B | C | B | B | A | A |
| 293 | B | C | B | B | A | A |
| 294 | B | B | B | B | B | B |
| 295 | B | B | B | B | A | A |
| 296 | B | B | B | B | A | B |
| 297 | B | B | B | B | A | A |
| 298 | B | B | B | B | A | A |
| 299 | B | C | C | B | A | A |
| 300 | B | C | C | B | A | B |
| 301 | B | C | C | B | A | A |
| 302 | B | C | B | B | A | A |
| 303 | B | B | B | B | A | B |
| 304 | C | C | C | B | A | B |
| 305 | B | B | B | B | A | A |
| 306 | C | D | C | C | B | B |
| 307 | C | C | C | C | A | A |
| 308 | B | C | C | B | B | C |
| 309 | B | B | B | B | A | A |
| 310 | B | C | B | B | A | B |
| 311 | D | D | D | D | C | C |
| 312 | B | B | B | B | A | A |
| 313 | B | B | B | B | A | A |
| 314 | B | B | B | B | A | A |
| 315 | B | C | C | B | A | A |
| 316 | B | C | B | B | A | A |
| 317 | B | C | B | B | A | A |
| 318 | B | C | B | C | A | B |
| 319 | B | C | C | C | B | B |
| 320 | B | C | C | B | A | A |
| 321 | B | B | B | B | A | A |
| 322 | B | C | B | B | A | B |
| 323 | B | B | B | B | A | A |
| 324 | B | B | B | B | A | A |
| 325 | B | B | B | B | A | A |
| 326 | B | C | B | B | A | B |
| 327 | B | B | B | B | A | A |
| 328 | B | B | B | B | A | A |
| 329 | B | B | B | B | A | A |
| 330 | B | C | C | B | A | B |
| 331 | A | B | A | B | A | A |
| 332 | A | B | A | A | A | A |
| 333 | A | B | A | A | A | A |
| 334 | D | D | D | D | B | B |
| 335 | A | A | A | A | A | A |
| 336 | C | D | D | C | B | B |
| 337 | B | B | B | B | A | A |
| 338 | B | B | B | B | A | A |
| 340 | B | B | B | B | A | B |
| 341 | B | C | B | B | A | B |
| 342 | C | C | C | B | A | B |
| 343 | C | C | D | C | B | B |
| 344 | C | C | D | B | A | B |
| 345 | C | D | D | C | B | B |
| 347 | A | B | B | B | A | B |
| 348 | B | C | B | B | A | B |
| 349 | A | B | A | B | A | A |
| 350 | B | B | B | B | A | A |
| 351 | B | C | B | B | A | A |
| 352 | C | D | D | D | B | A |
| 353 | B | C | C | C | A | A |
| 354 | C | D | C | C | B | A |
| 355 | B | B | B | B | A | A |
| 356 | A | B | B | B | A | A |
| 357 | B | C | B | B | A | B |
| 358 | C | C | C | C | A | A |
| 359 | B | C | B | B | A | A |
| 360 | C | C | C | C | A | B |
| 361 | B | C | B | B | A | A |
| 362 | B | C | B | B | A | B |
| 363 | B | B | B | B | A | A |
| 364 | B | C | C | C | A | A |
| 365 | B | B | B | B | A | A |
| 366 | B | C | C | B | A | A |
| 371 | B | B | B | B | A | A |
| 374 | B | C | C | B | A | A |
| 382 | B | C | C | B | A | A |
| 390 | B | A | B | B | A | A |
| 391 | C | C | C | C | A | A |
| 392 | B | B | B | B | A | A |
| 393 | C | D | D | D | A | A |
| 394 | A | A | A | A | A | A |
| 395 | A | A | A | A | A | A |
| 396 | B | B | B | B | A | A |
| 397 | A | B | B | B | A | A |
| 398 | B | B | B | B | A | A |
| 399 | B | B | B | B | A | A |
| 400 | B | C | B | B | A | B |
| 401 | B | B | B | B | A | A |
| 402 | B | B | B | C | A | A |
| 403 | B | B | B | B | A | A |
| 404 | C | C | D | A | A | A |
| 405 | B | B | B | B | A | A |
| 406 | B | B | B | B | A | A |
| 407 | B | B | B | B | A | A |
| 408 | B | C | C | B | A | B |
| 409 | B | B | B | B | A | A |
| 410 | B | B | B | B | A | A |
| 411 | B | B | B | B | A | A |
| 412 | B | B | B | B | A | A |
| 413 | D | D | D | D | C | C |
| 414 | B | B | B | B | A | B |
| 415 | B | C | B | B | A | A |
| 416 | B | C | C | B | A | B |
| 417 | B | C | C | B | A | B |
| 418 | B | C | C | B | A | B |
| 419 | B | B | B | B | A | A |
| 420 | C | C | C | C | B | B |
| 421 | C | C | C | C | B | B |
| 422 | C | C | C | C | B | B |
| 423 | B | B | B | B | A | A |
| 424 | B | C | B | B | A | B |
| 425 | B | C | B | B | A | A |
| 426 | C | C | C | C | B | B |
| 427 | B | B | B | B | A | A |
| 428 | C | C | C | B | A | A |
| 429 | B | C | C | C | A | A |
| 430 | B | C | B | B | A | A |
| 431 | C | C | C | C | A | A |
| 432 | C | C | B | B | A | B |
| 433 | C | C | C | C | B | B |
| 434 | C | C | C | B | B | B |
| 435 | B | B | B | B | A | A |
| 436 | C | C | B | C | A | A |
| 437 | B | B | B | B | A | A |
| 438 | B | C | B | B | A | A |
| 439 | B | C | C | C | A | A |
| 440 | B | B | B | B | A | A |
| 441 | C | C | C | B | A | A |
| 442 | B | B | B | B | A | A |
| 443 | B | B | B | B | A | A |
| 444 | B | C | C | B | A | A |
| 445 | C | D | D | D | C | B |
| 446 | A | B | B | B | A | A |
| 447 | B | B | B | B | A | A |
| 448 | B | C | C | B | A | B |
| 449 | C | C | C | C | B | B |
| 450 | B | B | B | B | A | A |
| 451 | B | C | C | B | A | B |
| 452 | B | B | B | B | A | B |
| 453 | C | C | C | C | A | A |
| 454 | B | B | B | B | A | A |
| 455 | B | C | B | B | A | B |
| 456 | B | B | B | B | A | A |
| 457 | B | B | B | B | A | A |
| 458 | B | B | B | B | A | A |
| 459 | B | B | B | B | A | A |
| 460 | B | B | B | B | A | A |
| 461 | A | B | B | A | A | A |
| 462 | B | B | B | B | A | A |
| 463 | A | A | A | A | A | A |
| 464 | A | A | A | B | A | A |
| 465 | A | B | B | B | A | A |
| 466 | B | B | B | B | A | A |
| 467 | A | A | A | B | A | A |
| 468 | D | D | D | D | B | C |
| 469 | A | A | A | A | A | A |
| 470 | A | A | A | A | A | A |
| 471 | B | B | B | B | A | A |
| 472 | B | C | C | C | A | A |
| 473 | B | B | B | B | A | A |
| 474 | B | B | B | B | A | A |
| 475 | B | B | B | B | A | A |
| 476 | B | B | B | B | A | A |
| 477 | B | B | B | B | A | A |
| 478 | B | C | C | B | A | A |
| 479 | B | B | B | B | A | A |
| 480 | C | C | C | C | A | A |
| 481 | B | C | B | B | A | B |
| 482 | B | B | B | B | A | A |
| 483 | C | D | D | D | A | A |
| 484 | C | C | C | C | A | A |
| 485 | C | C | C | C | A | A |
| 486 | B | B | B | B | A | A |
| 487 | B | C | C | B | A | A |
| 488 | A | B | A | B | A | A |
| 489 | B | B | B | B | A | A |
| 490 | B | C | C | C | B | B |
| 491 | C | D | D | C | B | B |
| 492 | A | B | A | A | A | A |
| 494 | B | C | B | B | A | A |
| 496 | B | C | C | C | A | B |
| 497 | B | C | C | C | A | A |
| 498 | D | D | D | D | C | C |
| 499 | B | C | C | C | A | A |
| 500 | B | B | B | B | A | B |
| 501 | A | B | A | B | A | A |
| 502 | B | B | B | B | A | B |
| 503 | B | C | C | C | A | B |
| 504 | D | D | D | D | C | C |
| 505 | C | C | C | C | A | B |
| 506 | B | C | C | C | A | A |
| 507 | B | C | B | C | A | A |
| 508 | B | B | B | B | A | A |
| 509 | B | B | B | B | A | A |
| 510 | C | C | B | C | A | A |
| 511 | B | B | C | B | A | A |
| 512 | B | B | B | B | A | A |
| 513 | B | B | B | B | A | B |
| 514 | B | B | B | B | A | A |
| 515 | B | B | B | B | A | A |
| 516 | A | B | A | B | A | A |
| 517 | A | A | A | B | A | A |
| 518 | A | A | A | A | A | A |
| 519 | A | B | A | A | A | A |
| 520 | B | B | B | B | A | A |
| 521 | A | A | A | B | A | A |
| 522 | B | B | A | A | A | A |
| 523 | A | A | A | A | A | A |
| 524 | A | A | A | A | A | A |
| 525 | C | C | C | B | A | A |
| 526 | B | B | B | B | A | A |
| 527 | B | B | B | B | A | A |
| 528 | C | C | C | C | A | B |
| 529 | A | A | A | A | A | A |
| 530 | B | B | B | B | A | A |
| 531 | B | B | B | B | A | A |
| 532 | B | B | B | B | A | A |
| 533 | B | B | B | B | A | A |
| 534 | B | B | B | B | A | A |
| 535 | C | C | C | C | A | A |
| 536 | C | C | C | C | A | A |
| 537 | A | A | A | A | A | A |
| 538 | A | A | A | A | A | A |
| 539 | A | A | A | A | A | A |
| 540 | A | A | A | A | A | A |
| 541 | A | A | A | A | A | A |
| 542 | A | A | A | A | A | A |
| 543 | A | A | A | A | A | A |
| 544 | B | B | B | B | A | A |
| 545 | B | C | C | B | A | A |
| 546 | A | A | A | A | A | A |
| 547 | A | A | A | A | A | A |
| 548 | A | A | A | A | A | A |
| 549 | A | B | B | B | A | A |
| 550 | A | B | A | A | A | A |
| 551 | A | A | A | A | A | A |
| 552 | A | A | A | A | A | A |
| 553 | A | A | A | A | A | A |
| 554 | A | A | A | A | A | A |
| 555 | A | A | A | A | A | A |
| 556 | A | B | A | B | A | A |
| 557 | A | B | A | B | A | A |
| 558 | B | B | B | B | A | A |
| 559 | A | A | A | B | A | A |
| 560 | A | A | A | A | A | A |
| 561 | A | A | A | A | A | A |
| 562 | A | B | A | A | A | A |
| 563 | A | B | B | B | A | A |
| 564 | B | C | B | B | A | A |
| 565 | A | B | A | B | A | A |
| 566 | B | B | B | B | A | A |
| 567 | B | B | B | B | A | A |
| 568 | C | B | B | C | A | A |
| 569 | B | B | A | B | B | B |
| 570 | A | A | A | A | A | A |
| 571 | A | A | A | A | A | A |
| 572 | A | B | A | A | A | A |
| 573 | A | A | A | A | A | A |
| 574 | A | A | A | A | A | A |
| 575 | A | A | A | A | A | A |
| 576 | A | B | A | A | A | A |
| 577 | A | A | A | A | A | A |
| 578 | A | A | A | A | A | A |
| 579 | A | A | A | A | A | A |
| 580 | A | A | A | A | A | A |
| 581 | A | A | A | A | A | A |
| 582 | A | A | A | A | A | A |
| 583 | A | A | A | A | A | A |
| 584 | A | A | A | A | A | A |
| 585 | A | A | A | A | A | A |
| 586 | A | B | A | A | A | A |
| 587 | A | A | A | A | A | A |
| 588 | B | B | B | B | A | A |
| 589 | B | B | B | B | A | A |
| 590 | A | A | A | A | A | A |
| 591 | A | A | A | A | A | A |
| 592 | C | C | C | C | A | B |
| 593 | C | C | C | C | B | B |
| 594 | C | C | C | C | A | A |
| 595 | C | C | C | C | A | A |
| 596 | A | B | B | B | A | A |
| 597 | B | B | B | A | A | A |
| 598 | A | A | A | A | A | A |
| 599 | A | A | A | A | A | A |
| 600 | A | A | A | A | A | A |
| 601 | B | B | B | B | A | A |
| 602 | C | C | C | C | A | A |
| 603 | A | A | A | A | A | A |
| 604 | A | A | A | A | A | A |
| 605 | A | B | A | A | A | A |
| 606 | A | A | A | A | A | A |
| 607 | A | A | A | A | A | A |
| 608 | A | B | B | B | A | A |
| 609 | A | A | A | A | A | A |
| 610 | A | B | B | B | A | A |
| 611 | A | A | A | A | A | A |
| 612 | A | A | A | A | A | A |
| 613 | B | B | B | B | A | A |
| 614 | A | A | A | A | A | A |
| 615 | A | A | A | A | A | A |
| 616 | A | A | A | A | A | A |
| 618 | A | A | A | A | A | A |
| 619 | A | A | A | A | A | A |
| 620 | A | A | A | B | A | A |
| 622 | A | A | A | A | A | A |
| 623 | B | B | B | B | A | B |
| 624 | B | B | B | B | A | A |
| 625 | B | C | C | C | A | A |
| 626 | B | B | B | B | A | A |
| 627 | A | A | A | B | A | A |
| 628 | A | A | A | A | A | A |
| 629 | B | B | B | B | A | A |
| 630 | B | B | B | B | A | A |
| 631 | A | A | A | A | A | A |
| 632 | B | B | B | B | A | A |
| 634 | A | A | A | A | A | A |
| 635 | A | B | A | A | A | A |
| 636 | A | A | A | A | A | A |
| 637 | A | A | A | A | A | A |
| 638 | A | A | A | A | A | A |
| 639 | A | A | A | A | A | A |
| 640 | A | A | A | A | A | A |
| 641 | C | C | C | B | A | A |
| 642 | C | C | C | C | A | B |
| 643 | C | C | C | C | B | B |
| 644 | C | D | D | C | B | B |
| 645 | B | B | B | B | A | A |
| 646 | A | A | A | B | A | A |
| 647 | A | A | B | B | A | A |
| 648 | A | A | A | B | A | A |
| 649 | B | B | B | B | A | A |
| 650 | B | B | B | B | A | A |
| 651 | A | A | A | A | A | A |
| 652 | A | A | A | A | A | A |
| 653 | A | A | A | A | A | A |
| 654 | A | A | A | A | A | A |
| 657 | A | A | A | A | A | A |
| 659 | A | A | A | A | A | A |
| 664 | A | A | A | A | A | A |
| 665 | B | B | B | B | A | A |
| 666 | A | A | A | A | A | A |
| 668 | B | B | B | B | A | A |
| 669 | B | B | B | B | A | A |
| 670 | B | B | B | B | A | A |
| 671 | B | B | B | B | A | B |
| 672 | B | B | B | B | A | A |
| 673 | A | A | A | A | A | A |
| 674 | A | A | A | A | A | A |
| 675 | A | A | A | A | A | A |
| 676 | A | A | A | A | A | A |
| 677 | A | A | A | A | A | A |
| 679 | A | A | A | A | A | A |
| 682 | A | A | A | A | A | A |
| 683 | A | A | A | A | A | A |
| 684 | A | A | A | A | A | A |
| 686 | A | A | A | B | A | A |
| 687 | A | A | A | A | A | A |
| 688 | A | B | A | B | A | A |
| 689 | A | A | A | B | A | A |
| 690 | A | A | A | A | A | A |
| 691 | A | A | A | A | A | A |
| 692 | A | A | A | A | A | A |
| 693 | A | A | A | A | A | A |
| 694 | A | B | B | B | A | A |
| 695 | B | B | B | B | A | A |
| 696 | A | A | A | A | A | A |
| 697 | A | A | A | A | A | A |
| 698 | B | B | B | B | A | A |
| 699 | B | B | B | B | A | A |
| 700 | A | B | A | A | A | A |
| 701 | C | C | C | D | A | A |
| 702 | C | C | C | C | A | B |
| 703 | B | C | C | B | A | A |
| 704 | A | A | A | A | A | A |
| 705 | A | B | A | A | A | A |
| 706 | B | B | B | B | A | A |
| 707 | A | A | A | A | A | A |
| 708 | A | A | A | A | A | A |
| 709 | A | A | A | A | A | A |
| 710 | A | B | A | A | A | A |
| 711 | A | A | A | A | A | A |
| 712 | A | A | A | A | A | A |
| 713 | B | B | B | B | A | A |
| 714 | B | B | B | B | A | A |
| 715 | A | A | A | A | A | A |
| 716 | C | D | D | C | A | B |
| 717 | B | B | B | B | A | A |
| 718 | A | A | A | A | A | A |
| 719 | A | A | A | A | A | A |
| 720 | A | A | A | A | A | A |
| 721 | A | A | A | A | A | A |
| 722 | A | A | A | A | A | A |
| 723 | A | A | A | A | A | A |
| 724 | A | A | A | A | A | A |
| 725 | A | A | A | A | A | A |
| 726 | A | A | A | A | A | A |
| 727 | A | B | A | B | A | A |
| 728 | C | C | C | C | B | B |
| 729 | A | A | A | A | A | A |
| 730 | A | A | A | A | A | A |
| 731 | B | B | B | B | A | B |
| 732 | A | A | A | B | A | A |
| 733 | A | B | B | B | A | A |
| 734 | B | B | B | B | A | A |
| 735 | A | A | A | A | A | A |
| 736 | B | B | B | C | A | B |
| 737 | A | A | A | A | A | A |
| 738 | B | B | B | B | A | A |
| 739 | A | B | A | A | A | A |
| 740 | B | B | B | B | A | A |
| 741 | A | B | B | B | A | A |
| 742 | B | B | B | C | A | A |
| 743 | B | B | B | B | A | A |
| 744 | B | B | B | B | A | A |
| 745 | A | A | A | A | A | A |
| 746 | A | A | A | A | A | A |
| 747 | B | B | B | B | A | A |
| 748 | A | A | A | A | A | A |
| 749 | A | A | A | A | A | A |
| 750 | A | A | A | A | A | A |
| 751 | A | A | A | B | A | A |
| 752 | A | B | A | B | A | A |
| 753 | B | B | B | B | A | A |
| 754 | A | B | A | B | A | A |
| 755 | A | B | A | B | A | A |
| 756 | A | A | A | A | A | A |
| 757 | A | A | A | A | A | A |
| 758 | A | A | A | A | A | A |
| 759 | A | A | A | A | A | A |
| 760 | A | A | A | A | A | A |
| 761 | A | A | A | A | A | A |
| 762 | A | A | A | A | A | A |
| 763 | B | B | B | B | A | A |
| 764 | A | A | A | A | A | A |
| 765 | A | A | A | A | A | A |
| 766 | A | A | A | A | A | A |
| 767 | A | B | B | B | A | A |
| 768 | A | A | A | A | A | A |
| 769 | A | A | A | A | A | A |
| 770 | A | A | A | A | A | A |
| 771 | A | A | A | A | A | A |
| 772 | A | A | A | A | A | A |
| 773 | B | B | B | B | A | A |
| 774 | B | B | B | B | A | A |
| 775 | A | A | A | A | A | A |
| 776 | A | B | A | B | A | A |
| 777 | A | B | A | B | A | A |
| 778 | A | A | A | A | A | A |
| 779 | A | B | B | B | A | A |
| 780 | A | A | A | A | A | A |
| 781 | A | B | A | A | A | A |
| 782 | B | B | B | B | A | A |
| 783 | B | B | B | B | A | A |
| 784 | A | B | A | A | A | A |
| 785 | A | B | B | B | A | A |
| 786 | A | B | A | B | A | A |
| 787 | A | A | A | A | A | A |
| 788 | A | A | A | A | A | A |
| 789 | A | B | A | A | A | A |
| 790 | A | B | A | A | A | A |
| 791 | A | A | A | A | A | A |
| 792 | A | A | A | A | A | A |
| 793 | A | A | A | A | A | A |
| 794 | A | A | A | A | A | A |
| 795 | A | A | A | A | A | A |
| 797 | A | A | A | A | A | A |
| 798 | B | C | C | C | B | B |
| 799 | C | C | C | B | B | B |
| 800 | A | A | A | A | A | A |
| 801 | A | B | B | B | A | A |
| 802 | A | A | A | B | A | A |
| 803 | A | B | B | B | A | A |
| 804 | A | A | A | A | A | A |
| 805 | A | A | A | A | A | A |
| 806 | A | B | A | A | A | A |
| 807 | A | A | A | A | A | A |
| 808 | A | A | A | A | A | A |
| 809 | A | A | A | A | A | A |
| 810 | A | A | A | A | A | A |
| 810 | A | A | A | A | A | A |
| 811 | A | B | B | B | A | A |
| 812 | A | B | B | B | A | A |
| 813 | A | B | B | B | A | A |
| 814 | A | B | A | B | A | A |
| 815 | A | A | A | A | A | A |

TABLE 12
Biological characterization of selected compounds of the present disclosure.

| | Raf1-GTP- | Raf1-GTP- | Raf1-GTP- | Raf1-GTP- | | |
|---|---|---|---|---|---|---|
| Compound | KRAS G12D | KRAS G12V | KRAS G12C | KRAS WT | pERK- | pERK- |
| No. | disruption | disruption | disruption | disruption | Gp2D | SW620 |
| 19 | A | B | | A | A | A |
| 145 | B | C | | C | A | A |
| 193 | B | B | | B | A | A |
| 198 | B | B | | B | A | A |
| 232 | B | B | | B | A | A |
| 245 | B | B | | B | A | A |
| 253 | B | C | | B | A | A |
| 262 | A | B | | B | A | A |
| 268 | B | B | | B | A | A |
| 275 | B | B | | B | A | A |
| 286 | B | B | | B | A | A |
| 287 | B | B | | B | A | A |
| 291 | B | B | | B | A | A |
| 298 | C | B | | B | A | A |
| 327 | B | B | | B | A | A |
| 328 | B | C | | B | A | A |
| 329 | B | B | | B | A | A |
| 331 | B | B | | B | A | A |
| 332 | B | B | | B | A | A |
| 333 | B | B | B | B | A | A |
| 335 | B | B | B | B | A | A |
| 355 | B | B | | C | A | A |
| 371 | B | C | C | B | A | A |
| 394 | B | B | B | B | A | A |
| 469 | A | A | A | A | A | A |
| 501 | B | B | B | B | A | A |
| 511 | C | C | C | B | A | A |
| 512 | B | C | B | B | A | A |
| 516 | B | B | B | B | A | A |
| 517 | B | B | B | B | A | A |
| 518 | A | A | A | A | A | A |
| 519 | A | B | B | B | A | A |
| 520 | B | B | B | B | A | A |
| 523 | A | A | A | A | A | A |
| 524 | A | A | B | A | A | A |
| 527 | B | B | B | B | A | A |
| 529 | A | A | A | A | A | A |
| 532 | B | B | B | B | A | A |
| 533 | B | B | B | B | A | A |
| 534 | B | B | B | B | A | A |
| 537 | A | A | B | B | A | A |
| 538 | A | A | A | B | A | A |
| 539 | B | B | B | B | A | A |
| 540 | A | A | A | B | A | A |
| 541 | A | A | A | B | A | A |
| 543 | A | A | A | A | A | A |
| 554 | A | B | B | B | A | A |
| 573 | A | A | A | A | A | A |
| 577 | A | A | A | A | A | A |
| 579 | A | B | | B | A | A |
| 581 | A | A | A | B | A | A |
| 586 | B | B | | B | A | A |
| 590 | B | B | | B | A | A |
| 600 | A | A | A | B | A | A |
| 603 | A | A | A | B | A | A |
| 605 | B | B | B | B | A | A |
| 606 | A | A | A | B | A | A |
| 607 | A | A | A | A | A | A |
| 608 | B | B | B | C | A | A |
| 610 | B | B | B | C | A | A |
| 611 | A | A | A | B | A | A |
| 613 | A | A | A | B | A | A |
| 614 | B | B | B | B | A | A |
| 615 | A | A | A | B | A | A |
| 618 | A | A | A | B | A | A |
| 619 | B | B | B | B | A | A |
| 622 | A | B | B | B | A | A |
| 631 | A | B | A | B | A | A |
| 636 | B | B | B | B | A | A |
| 638 | A | A | A | B | A | A |
| 646 | B | B | B | C | A | A |
| 660 | B | C | C | B | A | A |
| 661 | B | C | C | B | A | A |
| 662 | A | A | A | B | A | A |
| 663 | B | C | C | C | A | A |
| 667 | B | B | B | B | A | A |
| 673 | A | B | A | B | A | A |
| 674 | A | A | A | A | B | B |
| 683 | A | A | A | B | A | A |
| 689 | B | B | B | B | A | A |
| 692 | A | A | A | A | A | A |
| 696 | A | B | B | B | A | A |
| 697 | A | A | A | A | A | A |
| 702 | C | D | D | D | A | B |
| 703 | C | C | C | C | A | A |
| 704 | B | B | B | B | A | A |
| 705 | B | B | B | B | A | A |
| 706 | B | C | C | C | A | A |
| 726 | B | B | B | B | A | A |
| 732 | B | B | B | B | A | A |
| 741 | B | B | B | B | A | A |
| 744 | B | B | B | B | A | A |
| 749 | A | A | A | A | A | A |
| 752 | B | B | B | B | A | A |
| 764 | A | A | B | B | A | A |
| 765 | B | A | A | B | A | A |
| 796 | B | B | B | B | A | A |
| 816 | B | B | A | B | A | A |
| 817 | B | B | B | C | A | A |
| 818 | C | C | C | C | A | A |
| 822 | B | B | B | B | A | A |
| 823 | B | B | B | B | A | A |
| 824 | B | B | B | B | A | A |
| 825 | B | B | B | B | A | A |
| 826 | B | C | C | C | A | A |
| 827 | C | C | C | C | A | A |
| 828 | B | B | B | C | A | B |
| 829 | B | B | B | C | A | A |
| 830 | B | B | B | B | A | A |
| 831 | B | B | B | B | A | A |
| 832 | A | A | A | B | A | A |
| 833 | A | B | B | B | A | A |
| 834 | A | A | A | B | A | A |
| 835 | B | B | A | C | A | A |
| 836 | B | B | B | B | A | A |
| 837 | B | B | B | B | A | A |
| 838 | B | B | B | B | A | A |
| 839 | B | B | B | B | A | A |
| 840 | B | B | B | B | A | A |
| 841 | B | B | B | B | A | A |
| 842 | B | B | B | B | A | A |
| 843 | B | B | B | C | A | A |
| 844 | B | B | B | B | A | A |
| 845 | B | B | B | B | A | A |
| 846 | B | B | B | B | A | A |
| 847 | B | B | B | B | A | A |
| 848 | B | B | B | B | A | A |
| 849 | B | B | B | B | A | A |
| 850 | B | C | B | C | A | A |
| 851 | B | B | B | B | A | A |
| 852 | B | C | B | C | A | A |
| 853 | B | B | B | B | A | A |
| 854 | B | B | B | B | A | A |
| 855 | B | B | B | B | A | A |
| 856 | B | B | C | C | A | A |
| 857 | B | B | B | B | A | A |
| 858 | B | B | B | B | A | A |
| 859 | B | B | B | B | A | A |
| 860 | B | B | B | B | A | A |
| 861 | B | B | B | B | A | A |
| 862 | C | C | C | C | A | A |
| 863 | A | A | A | B | A | A |
| 864 | A | B | B | B | A | A |
| 865 | B | C | C | C | A | A |
| 866 | A | A | A | A | A | A |
| 867 | C | C | C | C | A | A |
| 868 | B | C | C | C | A | B |
| 869 | B | B | B | B | A | A |
| 870 | B | B | B | B | A | A |
| 871 | B | C | C | C | A | A |
| 872 | B | B | B | C | A | A |
| 873 | C | C | C | B | A | A |
| 874 | B | B | B | B | A | A |
| 875 | C | B | B | C | A | A |
| 876 | B | B | B | C | A | A |
| 877 | B | C | C | C | A | A |
| 878 | B | B | B | B | A | A |
| 879 | C | D | D | D | A | A |
| 880 | B | A | A | B | A | A |
| 881 | B | B | B | B | A | A |
| 882 | A | A | A | B | A | A |
| 883 | B | B | B | B | A | A |
| 884 | B | A | A | B | A | A |
| 885 | B | B | B | C | A | A |
| 886 | B | A | A | B | A | A |
| 887 | C | C | C | C | A | B |
| 888 | B | B | B | B | A | A |
| 889 | B | B | B | B | A | A |
| 890 | D | D | D | D | B | B |
| 891 | A | A | A | A | A | A |
| 892 | B | B | B | B | A | A |
| 893 | B | A | B | B | A | A |
| 894 | C | C | C | C | A | A |
| 895 | B | B | B | C | A | A |
| 896 | B | C | C | C | A | A |
| 897 | B | B | B | B | A | A |
| 898 | B | B | C | C | A | A |
| 899 | B | B | B | B | A | A |
| 900 | B | B | B | B | A | A |
| 901 | B | B | B | B | A | A |
| 902 | A | A | B | B | A | A |
| 903 | A | A | A | B | A | A |
| 904 | C | C | C | C | A | A |
| 905 | B | B | B | C | A | A |
| 906 | B | B | B | B | A | A |
| 907 | B | B | B | B | A | A |
| 908 | B | C | C | C | A | A |
| 909 | B | B | B | B | A | A |
| 910 | B | B | B | B | A | A |
| 911 | B | B | B | C | A | A |
| 912 | B | B | B | B | A | A |
| 913 | B | C | C | C | A | A |
| 914 | B | A | B | B | A | A |
| 915 | A | A | B | B | A | A |
| 916 | B | B | B | B | A | A |
| 917 | B | B | C | C | A | A |
| 918 | A | A | A | A | A | A |
| 919 | B | B | B | B | A | A |
| 920 | B | B | B | B | A | A |
| 921 | B | B | B | B | A | A |
| 922 | A | A | A | B | A | A |
| 923 | B | B | C | B | A | A |
| 924 | B | B | B | B | A | A |
| 925 | B | B | B | B | A | A |
| 926 | B | B | B | B | A | A |
| 927 | B | B | B | B | A | A |
| 928 | B | B | B | B | A | A |
| 929 | B | B | B | B | A | A |
| 930 | B | B | B | C | A | A |
| 931 | B | B | B | B | A | A |
| 932 | B | C | B | C | A | A |
| 933 | C | D | D | D | A | A |
| 934 | B | B | B | B | A | A |
| 935 | B | B | B | B | A | A |
| 936 | B | C | C | C | A | A |
| 937 | C | C | C | C | A | A |
| 938 | C | D | D | D | A | B |
| 939 | B | C | C | C | A | A |
| 940 | B | B | B | B | A | A |
| 941 | C | D | C | C | A | A |
| 942 | D | D | D | D | A | B |
| 943 | D | D | D | D | A | A |
| 944 | D | D | D | D | B | B |
| 945 | C | D | D | D | A | A |
| 946 | C | C | C | C | A | B |
| 947 | C | D | C | D | A | B |
| 948 | C | D | C | D | A | A |
| 949 | B | B | B | B | A | A |
| 950 | B | B | B | B | A | A |
| 951 | B | B | B | B | A | A |
| 952 | B | B | B | C | A | A |
| 953 | B | C | C | C | A | B |
| 954 | A | B | B | B | A | A |
| 955 | C | D | D | D | A | B |
| 956 | B | B | B | C | A | A |
| 957 | B | C | C | C | A | A |
| 958 | B | B | B | B | A | A |
| 959 | C | C | C | C | A | A |
| 960 | B | B | B | C | A | A |
| 961 | C | C | D | D | A | A |
| 962 | C | D | D | C | A | A |
| 963 | C | D | D | C | C | C |
| 964 | C | C | C | C | A | A |
| 965 | B | B | B | B | A | A |
| 966 | B | B | B | B | A | A |
| 967 | B | C | C | C | A | A |
| 968 | B | C | C | B | A | A |
| 969 | C | C | C | C | A | A |
| 970 | B | B | B | B | A | A |
| 971 | D | D | D | D | A | A |
| 972 | B | B | B | B | A | A |
| 973 | B | B | B | C | A | A |

TABLE 13
Biological characterization of selected compounds of the present disclosure in cell-based pERK HTRF assays.

| Cmpd. No. | AsPC-1 | SW1990 | HPAC | Capan-2 | RKN | H358 | HCT116 | KP-2 | H1573 | A549 | MKN | HT1080 |
|---|---|---|---|---|---|---|---|---|---|---|---|---|
| 19 | | A | A | A | A | A | A | | A | B | A | C |
| 147 | | | | A | | | | | | | | |
| 193 | | A | A | A | | A | A | A | A | A | A | C |
| 198 | | A | B | A | | A | A | | A | B | A | C |
| 232 | | A | A | A | | A | A | | A | B | | C |
| 234 | | A | B | A | | A | A | | A | A | A | C |
| 258 | | A | C | | | A | A | | A | A | A | C |
| 260 | | A | B | A | A | A | A | | A | A | A | C |
| 262 | B | A | A | A | A | A | A | A | A | A | A | C |
| 268 | B | A | A | A | A | A | A | B | A | A | A | C |
| 275 | B | A | B | A | A | A | A | B | A | A | A | C |
| 323 | | A | A | A | | A | A | | A | A | A | C |
| 332 | A | A | A | A | A | A | A | A | A | A | A | C |
| 337 | | A | A | A | | A | A | | A | B | A | C |
| 390 | A | A | A | A | | A | | | | A | A | C |
| 392 | C | A | A | B | A | A | A | B | B | B | A | C |
| 397 | A | A | A | A | | A | | | | | A | C |
| 398 | | A | B | A | | A | A | | A | A | A | C |
| 440 | C | A | A | B | B | B | A | B | B | B | A | C |
| 442 | B | A | A | A | A | A | A | B | B | B | A | C |
| 463 | B | A | B | A | | A | | | | | A | C |
| 467 | B | A | B | A | | A | | | | | A | C |
| 468 | C | C | C | C | | C | | | | | C | C |
| 469 | A | A | B | A | A | A | A | B | A | | A | C |
| 470 | A | A | B | A | | A | | | | | A | C |
| 484 | A | A | B | A | | A | | | | | A | C |
| 485 | B | A | B | A | | A | | | | | A | C |
| 492 | A | A | A | A | | A | | | | | A | C |
| 501 | A | A | A | A | A | A | B | B | B | | A | |
| 518 | A | A | B | A | A | A | | | | | A | |
| 519 | A | A | B | A | A | A | | | | | A | |
| 523 | B | A | A | A | | A | A | A | A | | A | C |
| 524 | B | A | A | A | | A | A | A | A | | A | C |
| 540 | B | A | A | A | | A | A | A | A | | A | C |
| 541 | A | A | A | A | A | A | A | B | A | | A | C |
| 543 | A | A | A | A | A | A | | | | | A | |
| 579 | A | A | C | A | A | A | | | | | A | |
| 584 | A | A | B | A | A | A | | | | | A | |
| 586 | A | A | B | A | A | A | | | | | A | |
| 590 | A | A | A | A | A | A | | | | | A | |
| 606 | A | A | A | A | A | A | | | | | A | |
| 610 | A | A | A | A | A | A | | | | | A | |
| 631 | A | A | A | A | A | A | | | | | A | |
| 674 | A | A | A | A | A | A | | | | | A | |
| 696 | A | A | | A | A | A | | | | | A | |
| 697 | A | A | A | A | A | A | | | | | A | |
| 704 | A | A | | A | A | A | | | | | A | |
| 732 | | A | A | A | A | A | A | | | | A | |
| 751 | | A | A | A | A | A | A | | | | A | |
| 759 | A | A | | A | A | A | | | | | A | |
| 764 | A | A | B | A | A | A | | | | | A | |

TABLE 14
Biological characterization of selected compounds of the present disclosure in 3D cell viability assays.

| Cmpd. No. | AsPC-1 | SW1990 | HPAC | Capan-2 | RKN | H358 | HCT116 | KP-2 | H1573 | A549 | MKN | HT1080 |
|---|---|---|---|---|---|---|---|---|---|---|---|---|
| 19 | | | B | B | A | A | A | | A | B | A | C |
| 147 | | B | B | | | A | B | | A | A | | C |
| 153 | | B | B | | | A | B | | B | A | | C |
| 193 | | | B | A | | A | A | | A | A | A | C |
| 198 | | | B | A | | A | A | | A | A | A | C |
| 232 | | A | C | | | A | A | | A | A | | C |
| 234 | | A | C | | | A | B | | A | A | | C |
| 258 | | A | B | | | A | B | | A | A | | C |
| 260 | | B | B | A | A | A | B | | A | A | | C |
| 262 | A | A | A | A | A | A | B | B | A | A | A | C |
| 268 | A | A | A | A | A | A | B | B | A | A | A | C |
| 275 | C | A | C | A | A | A | B | A | A | A | A | C |
| 323 | | A | A | A | | A | B | | A | A | A | C |
| 332 | A | A | B | A | A | A | B | A | A | A | A | C |
| 337 | | A | B | B | | A | B | | A | A | A | C |
| 390 | A | A | B | A | | A | B | | | | A | C |
| 392 | A | A | B | B | A | A | B | B | B | B | A | C |
| 397 | A | A | B | A | | A | | | | | A | C |
| 398 | | B | B | B | | A | B | | A | A | A | C |
| 440 | A | A | B | B | A | B | C | A | C | B | A | C |
| 442 | A | A | B | A | A | A | B | B | B | B | A | C |
| 463 | A | A | C | A | | A | | | | | A | C |
| 467 | A | A | C | A | | A | | | | | A | C |
| 469 | A | A | A | A | A | A | | | | | A | C |
| 470 | A | A | C | A | | A | | | | | A | C |
| 484 | A | A | B | A | | A | | | | | B | C |
| 485 | A | A | B | A | | A | | | | | B | C |
| 492 | A | A | A | A | | A | B | | | | A | C |
| 501 | A | A | B | A | A | A | | | | | A | |
| 518 | A | A | A | A | A | A | | | | | A | |
| 519 | A | A | B | A | A | A | | | | | A | |
| 523 | B | A | C | A | | A | | | | | A | C |
| 524 | B | A | C | A | | A | | | | | A | C |
| 540 | B | A | B | A | | A | | | | | A | C |
| 541 | A | A | A | A | A | A | B | | | | A | C |
| 543 | A | A | A | A | A | A | | | | | A | |
| 579 | A | A | B | A | A | A | | | | | A | |
| 584 | A | A | A | A | A | A | | | | | A | |
| 586 | A | A | A | A | A | A | | | | | A | |
| 590 | A | A | A | A | A | A | B | | | | A | |
| 606 | A | A | B | A | A | A | | | | | A | |
| 610 | A | A | B | A | A | A | | | | | A | |
| 631 | A | A | B | A | A | A | | | | | A | |
| 674 | A | A | A | A | A | A | | | | | A | |
| 696 | A | A | | A | A | A | B | | | | A | |
| 697 | A | A | A | A | A | A | B | | | | A | |
| 704 | A | A | | A | A | A | | | | | A | |
| 732 | A | A | A | A | A | A | B | | | | A | |
| 751 | A | A | A | A | A | A | B | | | | A | |
| 759 | A | A | | A | A | A | | | | | A | |
| 764 | A | A | C | A | A | A | | | | | A | |

It should be understood from the foregoing that, while particular implementations have been illustrated and described, various modifications may be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations, and equivalents. It is intended that the following claims define the scope of the invention and that methods and structures within the scope of these claims and their equivalents be covered thereby.

1-349. (canceled)

350. A compound having the structure: 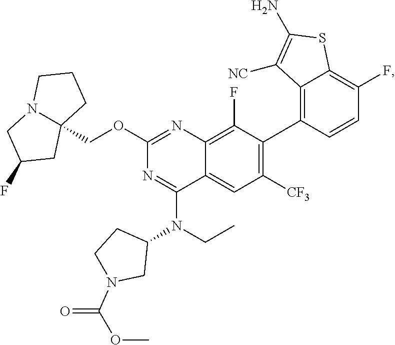 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@H]1CCN(C(=O)OC)C1]
or a pharmaceutically acceptable salt thereof.

351. A compound having the structure: 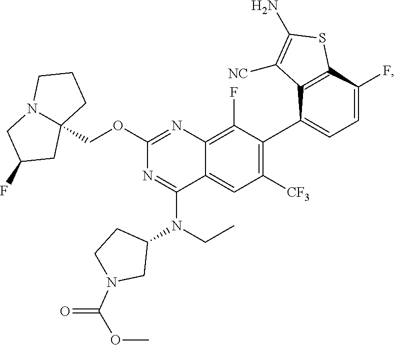
or an atropisomer thereof, a pharmaceutically acceptable salt thereof, or a pharmaceutically acceptable salt of the atropisomer thereof.

352. A compound having the structure: 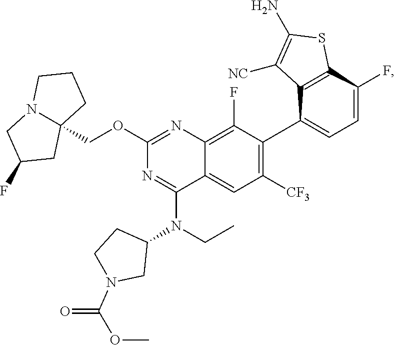
or a pharmaceutically acceptable salt thereof.

353. The compound of claim 350, wherein the compound is

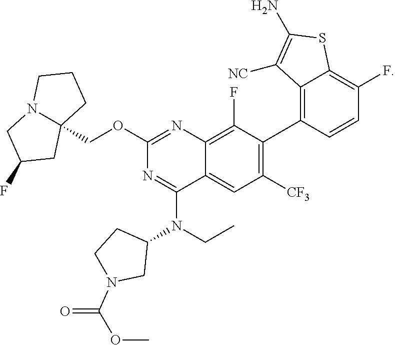 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@H]1CCN(C(=O)OC)C1]

354. The compound of claim 351, wherein the compound is 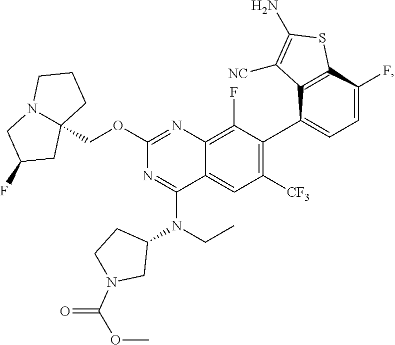
or an atropisomer thereof.

355. The compound of claim 352, wherein the compound is

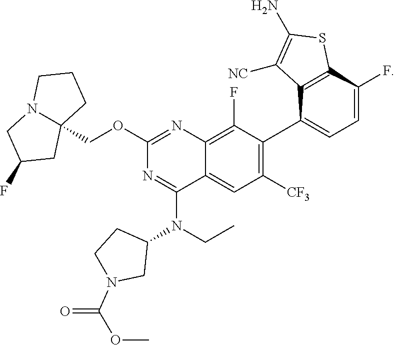

356. A pharmaceutical composition comprising a compound of claim 350, or a pharmaceutically acceptable salt thereof.

357. The pharmaceutical composition of claim 356, wherein the compound is

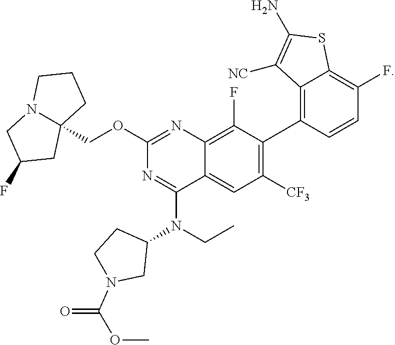 [SMILES: CCN(c1nc(OC[C@@]23CCCN2C[C@H](F)C3)nc2c(F)c(-c3ccc(F)c4sc(N)c(C#N)c34)c(C(F)(F)F)cc12)[C@H]1CCN(C(=O)OC)C1]

358. A pharmaceutical composition comprising a compound of claim 351, or an atropisomer thereof, a pharmaceutically acceptable salt thereof, or a pharmaceutically acceptable salt of the atropisomer thereof.

359. The pharmaceutical composition of claim 358, wherein the compound is 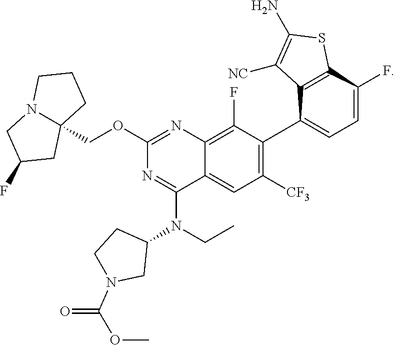
or an atropisomer thereof.

360. A pharmaceutical composition comprising a compound of claim 352, or a pharmaceutically acceptable salt thereof.

361. The pharmaceutical composition of claim 360, wherein the compound is

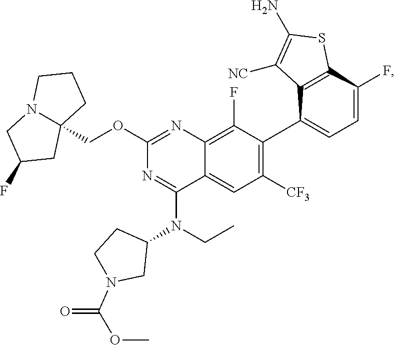

362. A compound selected from: 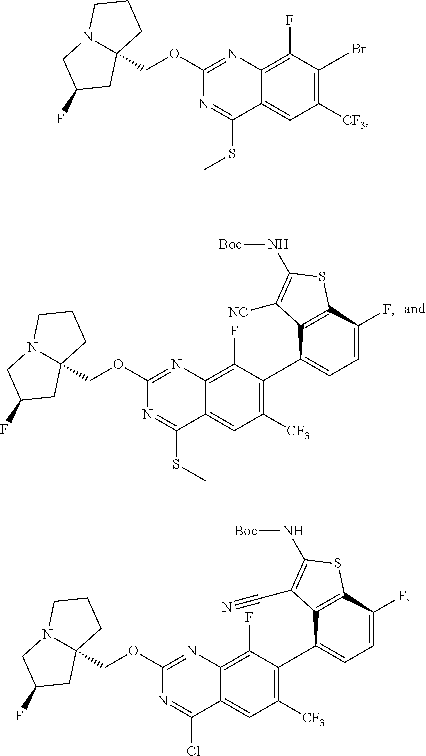
or a salt thereof.

363. The compound of claim 362, wherein the compound is 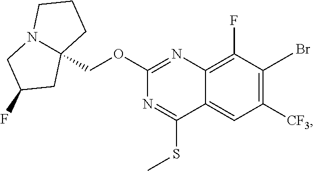 [SMILES: CSc1nc(OC[C@@]23CCCN2C[C@H](F)C3)nc2c(F)c(Br)c(C(F)(F)F)cc12]
or a salt thereof.

364. The compound of claim 363, wherein the compound is

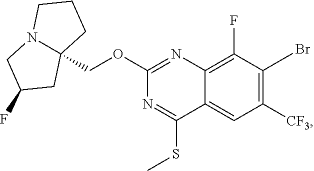 [SMILES: CSc1nc(OC[C@@]23CCCN2C[C@H](F)C3)nc2c(F)c(Br)c(C(F)(F)F)cc12]

365. The compound of claim 362, wherein the compound is 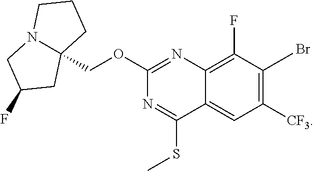 [SMILES: CSc1nc(OC[C@@]23CCCN2C[C@H](F)C3)nc2c(F)c(Br)c(C(F)(F)F)cc12]
or a salt thereof.

366. The compound of claim 365, wherein the compound is

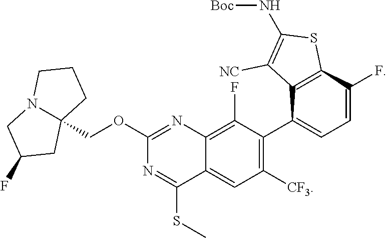

367. The compound of claim 362, wherein the compound is 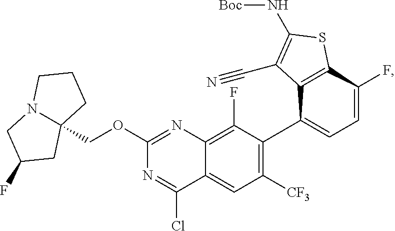
or a salt thereof.

368. The compound of claim 367, wherein the compound is [chemical structure]
